(19) United States
(12) Patent Application Publication
(10) Pub. No.: US 2026/0022142 A1
(43) Pub. Date: Jan. 22, 2026

(54) MACROCYCLIC IMMUNOMODULATORS

(71) Applicant: Bristol-Myers Squibb Company, Princeton, NJ (US)

(72) Inventors: Martin Patrick ALLEN, Princeton, NJ (US); Michael A. POSS, Princeton, NJ (US); Jennifer X. QIAO, Princeton, NJ (US); Claude A. QUESNELLE, Princeton, NJ (US); David R. TORTOLANI, Princeton, NJ (US); Tammy C. WANG, Princeton, NJ (US); Yunhui ZHANG, Princeton, NJ (US)

(21) Appl. No.: 18/867,716

(22) PCT Filed: May 19, 2023
(86) PCT No.: PCT/US2023/067255

§ 371 Date: Nov. 20, 2024

Related U.S. Application Data
(60) Provisional application No. 63/344,279, filed on May 20, 2022.

(51) Int. Cl. C07K 7/56; A61K 38/12

(52) U.S. Cl. CPC C07K 7/56; A61K 38/12

(57) ABSTRACT

In accordance with the present disclosure, macrocyclic compounds have been discovered that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer and infectious diseases.

### CROSS-REFERENCE TO RELATED APPLICATIONS

This application claims the priority benefit of U.S. Provisional Application No. 63/344,279, filed May 20, 2022, which is incorporated herein by reference in its entirety.

### FIELD

The present disclosure provides macrocyclic compounds that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer.

### BACKGROUND

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al., Curr. Opin. Immunol., 14:779-782 (2002); Bennett et al., J. Immunol., 170:711-718 (2003)).

The PD-1 protein is a 55 kDa type I transmembrane protein that is part of the Ig gene superfamily (Agata et al., Int. Immunol., 8:765-772 (1996)). PD-1 contains a membrane proximal immunoreceptor tyrosine inhibitory motif (ITIM) and a membrane distal tyrosine-based switch motif (ITSM) (Thomas, M. L., J. Exp. Med., 181:1953-1956 (1995); Vivier, E. et al., Immunol. Today, 18:286-291 (1997)). Although structurally similar to CTLA-4, PD-1 lacks the MYPPY motif that is critical for CD80 CD86 (B7-2) binding. Two ligands for PD-1 have been identified, PD-L1 (B7-H1) and PD-L2 (b7-DC). The activation of T cells expressing PD-1 has been shown to be downregulated upon interaction with cells expressing PD-L1 or PD-L2 (Freeman et al., J. Exp. Med., 192:1027-1034 (2000); Latchman et al., Nat. Immunol., 2:261-268 (2001); Carter et al., Eur. J. Immunol., 32:634-643 (2002)). Both PD-L1 and PD-L2 are B7 protein family members that bind to PD-1, but do not bind to other CD28 family members. The PD-L1 ligand is abundant in a variety of human cancers (Dong et al., Nat. Med., 8:787-789 (2002)). The interaction between PD-1 and PD-L1 results in a decrease in tumor infiltrating lymphocytes, a decrease in T-cell receptor mediated proliferation, and immune evasion by the cancerous cells (Dong et al., J. Mol. Med., 81:281-287 (2003); Blank et al., Cancer Immunol. Immunother., 54:307-314 (2005); Konishi et al., Clin. Cancer Res., 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad. Sci. USA, 99:12293-12297 (2002); Brown et al., J. Immunol., 170:1257-1266 (2003)).

When PD-1 expressing T cells contact cells expressing its ligands, functional activities in response to antigenic stimuli, including proliferation, cytokine secretion, and cytotoxicity, are reduced. PD-1/PD-L1 or PD-L2 interactions down regulate immune responses during resolution of an infection or tumor, or during the development of self tolerance (Keir, M. E. et al., Annu. Rev. Immunol., 26: Epub (2008)). Chronic antigen stimulation, such as that which occurs during tumor disease or chronic infections, results in T cells that express elevated levels of PD-1 and are dysfunctional with respect to activity towards the chronic antigen (reviewed in Kim et al., Curr. Opin. Imm. (2010)). This is termed “T cell exhaustion”. B cells also display PD-1/PD-ligand suppression and “exhaustion”.

In addition to enhancing immunologic responses to chronic antigens, blockade of the PD-1/PD-L1 pathway has also been shown to enhance responses to vaccination, including therapeutic vaccination in the context of chronic infection (Ha, S. J. et al., “Enhancing therapeutic vaccination by blocking PD-1-mediated inhibitory signals during chronic infection”, J. Exp. Med., 205 (3): 543-555 (2008); Finnefrock, A. C. et al., “PD-1 blockade in rhesus macaques: impact on chronic infection and prophylactic vaccination”, J. Immunol., 182 (2): 980-987 (2009); Song, M.-Y. et al., “Enhancement of vaccine-induced primary and memory CD8+t-cell responses by soluble PD-1”, J. Immunother., 34 (3): 297-306 (2011).

The PD-1 pathway is a key inhibitory mechanism in T cell exhaustion that arises from chronic antigen stimulation during tumor disease. Accordingly, agents that block the interaction of PD-1 with PD-L1 are desired.

### SUMMARY

The present disclosure provides macrocyclic compounds which inhibit the PD-1/PD-L1 protein/protein interaction, and are thus useful for the amelioration of various diseases, including cancer.

In certain aspects, the present disclosure provides a compound of formula (I):

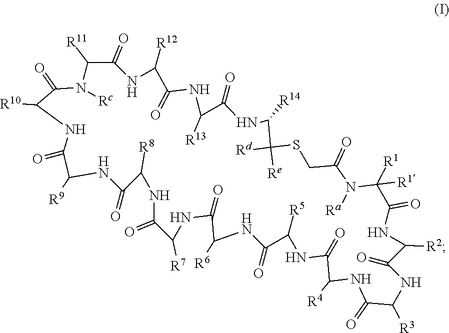

or a pharmaceutically acceptable salt thereof, wherein:
R¹ is selected from C₁-C₆alkyl, C₁-C₂alkylaminoC₁-C₆alkyl, C₁-C₆alkylcarbonylaminoC₁-C₆alkyl, C₁-C₆alkylheteroarylC₁-C₆alkyl, C₁-C₆alkylimidazolylC₁-C₂alkyl, aminoC₁-C₆alkyl, aminocarbonylC₁-C₆alkyl, aminocarbonylaminoC₁-C₆alkyl, arylC₁-C₂alkyl, biarylC₁-C₆alkyl optionally substituted with carboxy, guanidinylC₂-C₆alkyl, carboxyC₁-C₆alkyl, C₃-C₆cycloalkyl, (C₃-C₆cycloalkyl) C₁-C₆alkyl, C₃-C₆cycloalkylcarbonylaminoC₁-C₆alkyl, fluoroheterocyclylC₁-C₆alkyl, heterocyclylC₁-C₆alkyl, heteroarylcarbonylaminoC₁-C₆alkyl, heteroarylC₁-C₆alkyl, hydroxyC₁-C₆alkyl, and methoxyC₁-C₂alkyl; wherein the aryl part of the arylC₁-C₂alkyl is optionally substituted with one, two, or three groups independently selected from amino, aminocarbonyl, carboxy, carboxyC₁-C₆alkyl, carboxymethoxy, cyano, fluoro, hydroxy, methoxy, methyl, methylcarbonylamino, and trifluoromethyl;
R^{1′} is hydrogen, or, R¹ and R^{1′}, together with the carbon atom to which they are attached, form a cyclopropyl ring;
R² is selected from arylC₁-C₂alkyl, carboxyC₁-C₆alkyl, (C₃-C₆cycloalkyl) C₁-C₆alkyl, guanidinylC₂-C₆alkyl, heteroarylC₁-C₆alkyl, heterocyclylC₁-C₆alkyl, and hydroxyC₁-C₆alkyl, wherein the aryl part of the arylC₁-C₂alkyl is optionally substituted with one, two, or three groups independently selected from aminocarbonyl, carboxy, carboxyC₁-C₆alkoxy, cyano, fluoro, hydroxy, methyl, methoxy, —SO₃H, and trifluoromethoxy;
R³ is carboxymethyl;
R⁴ is selected from arylC₁-C₂alkyl, benzothienylC₁-C₂alkyl, heteroarylC₁-C₆alkyl, indolylC₁-C₆alkyl, and naphthalenylC₁-C₂alkyl; wherein the aryl part of the arylC₁-C₂alkyl is optionally substituted with one, two, three, four, or five groups independently selected from amino, carboxyC₁-C₆alkyl, chloro, cyano, fluoro, hydroxy, methoxy, methyl, and trifluoromethyl;
R⁵ is selected from C₁-C₆alkyl, C₁-C₆alkylcarbonylaminoC₁-C₆alkyl, aminocarbonylaminoC₂-C₆alkyl, aryl, arylC₁-C₂alkyl, benzothienylC₁-C₂alkyl, carboxyC₁-C₆alkyl, C₃-C₆cycloalkyl, (C₃-C₆cycloalkyl) C₁-C₆alkyl, C₃-C₆cycloalkylcarbonylaminoC₁-C₆alkyl, fluoroC₁-C₆alkyl, heteroarylC₁-C₆alkyl, heterocycloalkylC₁-C₆alkyl, and hydroxyC₁-C₆alkyl; wherein the aryl part of the arylC₁-C₂alkyl is optionally substituted with one, two, three, four, or five groups independently selected from amino, aminocarbonyl, aminomethyl, carboxy, carboxyC₁-C₆alkyl, carboxymethoxy, cyano, fluoro, hydroxy, methoxy, methyl, methylcarbonylamino, trifluoromethoxy, and trifluoromethyl;
R⁶ is biarylC₁-C₆alkyl;
R⁷ is selected from fluoroC₁-C₆alkylcarbonylaminoC₁-C₆alkyl, C₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, arylC₁-C₂alkyl, carboxyC₁-C₆alkyl, (C₃-C₆cycloalkyl) C₁-C₆alkyl, fluoroC₁-C₆alkyl, guanidinylC₂-C₄alkyl, heteroarylC₁-C₆alkyl, C₃-C₆heterocycloalkylC₁-C₆alkyl, hydroxyC₁-C₆alkyl, and methylsulfanylC₁-C₆alkyl; wherein the aryl part of the arylC₁-C₂alkyl is optionally substituted with one, two, three, four, or five groups independently selected from amino, aminocarbonyl, aminoC₁-C₆alkyl, aminocarbonyl, aminomethyl, carboxy, carboxyC₁-C₆alkyl, chloro, fluoro, methyl, hydroxy, methylcarbonylamino, and trifluoromethyl;
R⁸ is selected from C₁-C₂alkylcarbonylaminoC₁-C₆alkyl, aminoC₁-C₆alkyl, aminocarbonylC₁-C₆alkyl, aminocarbonylaminoC₁-C₆alkyl, carboxyC₁-C₆alkyl, guanidinylC₁-C₆alkyl, and heteroarylC₁-C₆alkyl;
R⁹ is C₁-C₆alkyl or (C₃-C₆cycloalkyl) C₁-C₂alkyl;
R¹⁰ is selected from aminoC₁-C₆alkyl, carboxyC₁-C₆alkyl, hydroxyC₁-C₆alkyl, aminocarbonylC₁-C₂alkyl, guanidinylC₁-C₆alkyl, and heteroarylC₁-C₆alkyl;
R¹¹ is selected from C₂-C₆alkyl, arylC₁-C₂alkyl, and (C₃-C₆cycloalkyl) C₁-C₂alkyl; wherein the aryl part of the arylC₁-C₂alkyl is optionally substituted with one, two, three, four, or five groups independently selected from chloro, fluoro, methyl, and trifluoromethyl;
R¹² is selected from C₃-C₆alkyl, aminoC₁-C₆alkyl, fluoroC₄-C₆alkyl, heteroarylC₁-C₆alkyl, hydroxyC₁-C₆alkyl, and hydroxyarylC₁-C₂alkyl;
R¹³ is selected from C₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, aryl, arylC₁-C₂alkyl, carboxyC₁-C₆alkyl, cyanoC₁-C₆alkyl, guanidinylC₁-C₆alkyl, C₂-C₆alkynylmethoxy, heteroaryl, heteroarylC₁-C₆alkyl, C₃-C₆heterocyclylC₁-C₆alkyl, hydroxyC₁-C₆alkyl, and hydroxyarylC₁-C₂alkyl; wherein the aryl part of the arylC₁-C₂alkyl is optionally substituted with one, two, three, four, or five groups independently selected from carboxyC₁-C₆alkyl, ethynylmethoxy, and hydroxy;
   R¹⁴ is —C(O)NR^{14′}CR¹⁵R^{15′}R^{15″}, —C(O)NH(CH₂)_{o′}Ph(CH₂)_{o′}C(O)NHCHR¹⁷R^{17′}, —C(O)NH(CH₂)_{o′}cyclopropyl(CH₂)_{o′}C(O)NHCHR¹⁷R^{17′}, or —C(O)NR⁵⁰R⁵¹, wherein:
      R⁵⁰ and R⁵¹, together with the nitrogen atom to which they are attached, form a piperazine ring, wherein the ring is further substituted with one —(CH₂)_{o′}C(O)NHCHR¹⁷R^{17′} group;
   o′ is 0, 1, or 2;
      R^{14′} is hydrogen or C₁-C₆alkyl, or R¹⁵ and R^{14′}, together with the atoms to which they are attached, form a morpholine, piperazine, or piperidine ring;
      R¹⁵ is selected from hydrogen, C₂-C₆alkenyl, C₁-C₁₆alkyl, C₁-C₆alkylcarbonylaminoC₁-C₆alkyl, C₂-C₆alkynyl, aminoC₁-C₆alkyl, aminocarbonylC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, and hydroxyC₁-C₆alkyl;
      R^{15′} is hydrogen or R¹⁵ and R^{15′}, together with the atoms to which they are attached, form a C₃-C₈cycloalkyl ring; and
   R^{15″} is —(CH₂)ₘCO₂H CH₂O((CH₂)₂O)ₙCH₂C(O)NHCHR¹⁶R^{16′} or —C(O)NHCHR¹⁶R^{16′}; wherein:
      R¹⁶ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
      R^{16′} is —(CH₂)ₘCO₂H, —CH₂O((CH₂)₂O)ₙCH₂C(O)NR⁷⁵CR^{17″}R¹⁷R^{17′}, -Ph(CH₂)_{o′}C(O)NHCHR¹⁷R¹⁷, or —(CH₂)ₒC(O)NHCHR¹⁷R^{17′}; wherein:
         R⁷⁵ is hydrogen;
         R¹⁷ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; or R¹⁷ and R⁷⁵, together with the atoms to which they are attached, form a pyrrolidine ring;
         R^{17′} is —CH₂O((CH₂)₂O)ₙCH₂C(O)NHCHR¹⁸R^{18′}, —(CH₂)ₘCO₂H or —(CH₂)ₘC(O)NHR¹⁸R^{18′}; and
         R^{17″} is hydrogen, or R^{17″} and R¹⁷ form a C₃-C₈ cycloalkyl ring; wherein:
         R¹⁸ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R¹⁸ is —(CH₂)ₘCO₂H, —(CH₂)ₘC(O)NR¹⁹R^{19′}, or —CH₂O((CH₂)₂O)ₙCH₂C(O)NHCHR¹⁹R^{19′}; wherein:
         R¹⁹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl;
         R^{19′} is —(CH₂)ₘC(O)NR¹⁹R^{19′}, —(CH₂)ₘCO₂H, or —CH₂O((CH₂)₂O)ₙCH₂C(O)NHCHR²⁰R^{20′}, wherein:
         R²⁰ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{20′} is —(CH₂)ₘCO₂H or —(CH₂)ₘC(O)NR²¹R^{21′}; wherein:
         R²¹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{21′} is —(CH₂)ₘCO₂H or —(CH₂)ₘC(O)NR²²R²²; wherein:
         R²² is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R²² is —(CH₂)ₘCO₂H; wherein
      m is a integer from 1 to 10;
      n is 1, 2, or 3; and
      o′ is 0, 1, or 2;
R^{a} is hydrogen or C₁-C₆alkyl;
R^{c} is hydrogen or C₁-C₆alkyl;
R^{d} is hydrogen or C₁-C₆alkyl; and
R^{e} is hydrogen or C₁-C₆alkyl.

In some aspects, R¹ is C₁-C₆alkyl, aminocarbonylC₁-C₃alkyl, aminoC₁-C₆alkyl, arylC₁-C₂alkyl, guanidinylC₂-C₆alkyl, heteroarylC₁-C₆alkyl, hydroxyC₁-C₆alkyl, biarylC₁-C₆alkyl optionally substituted with carboxy and methoxyC₁-C₂alkyl; wherein the aryl part of the arylC₁-C₂alkyl is optionally substituted with one, two, or three groups independently selected from amino, aminocarbonyl, carboxy, carboxyC₁-C₆alkyl, carboxymethoxy, cyano, fluoro, hydroxy, methyl, methylcarbonylamino, and trifluoromethyl.

In some aspects, R¹ is C₁-C₄alkyl, aminoC₁-C₃alkyl, aminocarbonylmethyl, benzyl, guanidinylpropyl, hydroxyC₁-C₃alkyl, imidazolylmethyl, methoxymethyl, morpholinylmethyl, and pyridinylmethyl; wherein the benzyl is optionally ring substituted with one, two, or three groups independently selected from aminocarbonyl, carboxy, carboxymethoxy, cyano, fluoro, and trifluoromethyl.

In some aspects, R² is arylC₁-C₂alkyl, optionally ring substituted with one, two, or three groups independently selected from aminocarbonyl, carboxy, carboxyC₁-C₆alkoxy, cyano, fluoro, methyl, hydroxy, —SO₃H, and trifluoromethoxy.

In some aspects, R² is benzyl, optionally ring substituted with one, two, or three groups independently selected from carboxy, carboxyC₁-C₆alkoxy, cyano, and hydroxy.

In certain aspects, R³ is carboxymethyl.

In some aspects, R⁴ is selected from arylC₁-C₂alkyl, heteroarylC₁-C₆alkyl, and indolylC₁-C₂alkyl; wherein the aryl part of the arylC₁-C₂alkyl are optionally substituted with one or more groups independently selected from amino, carboxyC₁-C₆alkyl, chloro, cyano, fluoro, hydroxy, methoxy, methyl, and trifluoromethyl.

In some aspects, R⁴ is selected from arylmethyl, heteroarylmethyl, and indolylmethyl; wherein the aryl part of the arylmethyl are optionally substituted with one or more groups independently selected from methyl and trifluoromethyl.

In some aspects, R⁵ is C₁-C₆alkyl or arylC₁-C₂alkyl; wherein the aryl part of the arylC₁-C₂alkyl is optionally substituted with one, two, three, four, or five groups independently selected from amino, aminocarbonyl, aminomethyl, cyano, carboxy, carboxyC₁-C₆alkyl, carboxymethoxy, fluoro, hydroxy, methoxy, methyl, methylcarbonylamino, trifluoromethyl, and trifluoromethoxy.

In some aspects, R⁵ is isopropyl or a benzyl optionally ring substituted with one, two, three, four, or five groups independently selected from amino, aminocarbonyl, carboxy, carboxymethoxy, cyano, fluoro, hydroxy, methyl, and trifluoromethyl.

In some aspects, R⁶ is biarylC₁-C₆alkyl.

In some aspects, R⁷ is selected from C₄-C₆alkyl, C₁-C₆alkylcarbonylaminoC₁-C₆alkyl, aminocarbonylC₁-C₆alkyl, aminocarbonylaminoC₁-C₆alkyl, and arylC₁-C₂alkyl; wherein the aryl part of the arylC₁-C₂alkyl is optionally substituted with one, two, three, four, or five groups independently selected from amino, chloro, fluoro, hydroxy, methyl, aminocarbonyl, aminoC₁-C₆alkyl, aminocarbonyl, aminomethyl, methylcarbonylamine, carboxy, hydroxy, and carboxyC₁-C₆alkyl.

In some aspects, R⁷ is selected from aminocarbonylethyl, aminocarbonylaminopropyl, benzyl, isopentenyl, and methylcarbonylaminobutyl, wherein the benzyl is optionally ring substituted with one, two, three, four, or five groups independently selected from carboxy, carboxyC₁-C₆alkyl, hydroxy, and trifluoromethyl.

In some aspects, R⁸ is aminoC₁-C₆alkyl or heteroarylC₁-C₆alkyl.

In some aspects, R⁸ is selected from aminobutyl, aminoethyl, aminopropyl, and imidazolylmethyl.

In some aspects, R⁹ is C₁-C₆alkyl and the stereochemistry of the center to which R^{o} is attached is R-stereochemistry.

In some aspects, R⁹ is —(CH₂)CH(CH₃)₂.

In some aspects, R¹⁰ is aminoC₁-C₆alkyl or heteroarylC₁-C₆alkyl.

In some aspects, R¹⁰ is aminoethyl or imidazolylmethyl.

In some aspects, R¹¹ is (C₃-C₆cycloalkyl) C₁-C₂alkyl.

In some aspects, R¹¹ is C₆cycloalkylmethyl.

In some aspects, R¹² is selected from C₄-C₆alkyl, fluoroC₄-C₆alkyl, hydroxyC₁-C₆alkyl, and hydroxyarylC₁-C₂alkyl.

In some aspects, R¹² is selected from fluoroisopropyl, hydroxyisopropyl, hydroxyethyl, and isopropyl.

In some aspects, R¹³ is selected from C₁-C₆alkyl, aminoC₁-C₆alkyl, aminocarbonylC₁-C₆alkyl, aminocarbonylaminoC₁-C₆alkyl, carboxyC₁-C₆alkyl, guanidinylC₁-C₆alkyl, heteroaryl, heteroarylC₁-C₆alkyl, and hydroxyC₁-C₆alkyl.

In some aspects, R¹³ is selected from aminobutyl, aminocarbonylaminopropyl, aminocarbonylethyl, aminoethyl, aminopropyl, carboxyethyl, carboxymethyl, guanidinylpropyl, hydroxyethyl, hydroxymethyl, and imidazolylmethyl.

In some aspects, the present disclosure provides a compound of formula (I), or the pharmaceutically acceptable salt thereof, wherein
R¹ is selected from aminobutyl, aminocarbonylaminopropyl, aminoethyl, aminomethyl, aminocarbonylethyl, aminocarbonylmethyl, arylmethyl, azetidinylmethyl, butyl, carboxyethyl, cyclobutylmethyl, cyclohexylmethyl, cyclopropyl, cyclopropycarbonylaminoethyl, cyclopropylcarbonylaminopropyl, difluorocyclohexylmethyl, ethyl, furanylmethyl, guanidinylbutyl, guanidinylpropyl, hydroxyethyl, hydroxyisopropyl, hydroxymethyl, imidazolylmethyl, isopentenyl, isopropylmethyl, methyl, methylaminomethyl, methylcarbonylaminobutyl, methylimidazolylmethyl, methoxymethyl, morpholinylmethyl, propyl, pyridinylmethyl, trimethylcarbonylaminoethyl, trimethylcarbonylaminobutyl, and trimethylcarbonylaminomethyl; wherein the aryl part of the arylmethyl is optionally substituted with one, two, or three groups independently selected from amino, aminocarbonyl, carboxy, carboxymethoxy, cyano, fluoro, hydroxy, methyl, methylcarbonylamino, and trifluoromethyl;
R² is selected from arylmethyl, carboxyethyl, guanidinylpropyl, imidazolylmethyl, and pyridinylmethyl; wherein the aryl part of the arylmethyl is optionally substituted with one, two, or three groups independently selected from carboxy, carboxyC₁-C₆alkoxy, cyano, fluoro, methyl, hydroxy, —SO₃H, and trifluoromethoxy;
R³ is carboxymethyl;
R⁴ is selected from arylmethyl, benzothienylmethyl, indolylmethyl, heteroarylmethyl, and naphthalenylmethyl; wherein the aryl part of the arylmethyl are optionally substituted with one or more groups independently selected from amino, chloro, cyano, fluoro, hydroxy, methyl, and trifluoromethyl;
R⁵ is selected from aminocarbonylaminopropyl, aryl, arylmethyl, arylpropyl, carboxyethyl, cyclopropyl, cyclopropylcarbonylaminobutyl, fluoroisopropyl, hydroxymethyl, hydroxyisopropyl, indolylmethyl, isopentenyl, isopentenylmethyl, isopropyl, methylcarbonylaminobutyl, methylcarbonylaminoethyl, pyridinylmethyl, tert-butylcarbonylaminobutyl, tert-butylcarbonylaminoethyl, tert-butyl, and thiophenylmethyl; wherein the aryl part of the arylmethyl is optionally substituted with one, two, three, four, or five groups independently selected from amino, aminocarbonyl, aminomethyl, carboxy, carboxymethoxy, cyano, fluoro, methoxy, methyl, methylcarbonylamino, hydroxy, and trifluoromethyl;
R⁶ is biarylC₁-C₃alkyl;
R⁷ is selected from aminocarbonylaminopropyl, aminocarbonylethyl, aminomethyl, arylmethyl, carboxyethyl, carboxybutyl, aminobutyl, aminopropyl, cyclohexylmethyl, fluoroisopropyl, guanidinylpropyl, hydroxyisopropyl, hydroxymethyl, imidazolylmethyl, isopentenyl, isopropyl, methylcarbonylaminobutyl, methylsulfanylethyl, pyridinylmethyl, tert-butylcarbonylaminobutyl, and tert-butylcarbonylaminopropyl, wherein the aryl part of the arylmethyl is optionally substituted with one, two, three, four, or five groups independently selected from amino, aminoC₁-C₆alkyl, aminocarbonyl, aminomethyl, carboxy, carboxyC₁-C₆alkyl, chloro, fluoro, hydroxy, methyl, methylcarbonylamino, hydroxy, and trifluoromethyl;
R⁸ is selected from aminobutyl, aminocarbonylaminopropyl, aminocarbonylethyl, aminoethyl, aminomethyl, aminopropyl, carboxyethyl, guanidinylpropyl, imidazolylmethyl, and methylcarbonylaminobutyl;
R⁹ is selected from butyl, cyclopropylmethyl, cyclobutylmethyl, cyclohexylmethyl, cyclopentylmethyl, isopropylmethyl, and methyl;
R¹⁰ is selected from aminocarbonylmethyl, aminoethyl, aminomethyl, carboxymethyl, guanidinylpropyl, hydroxyethyl, and imidazolylmethyl;
R¹¹ is selected from arylmethyl, butyl, cyclobutylmethyl, cyclohexylethyl, cyclohexylmethyl, cyclopropylmethyl, isopentenyl, and pentyl; wherein the aryl part of the arylmethyl is optionally substituted with one, two, three, four, or five groups independently selected from chloro, fluoro, hydroxy, methyl, and trifluoromethyl;
R¹² is selected from aminoethyl, aminobutyl, fluoroisopropyl, hydroxyarylmethyl, hydroxyethyl, hydroxyisopropyl, hydroxymethyl, imidazolylmethyl, isopentenyl, isopropyl, and trimethylmethyl;
R¹³ is selected from, aminobutyl, aminocarbonylaminopropyl, aminocarbonylbutyl, aminocarbonylmethyl, aminocarbonylpropyl, aminocarbonylethyl, aminoethyl, aminomethyl, aminopropyl, aryl, arylmethyl, butyl, carboxyethyl, carboxymethyl, cyanoethyl, cyanomethyl, furanylmethyl, guanidinylpropyl, hydroxyarylmethyl, hydroxyethyl, hydroxyisopropyl, hydroxymethyl, hydroxypropyl, imidazolylmethyl, methylcarbonylaminobutyl, methylcarbonylaminoethyl, pyridinyl, tetrahydropyranyl, tetrahydropyranylmethyl, tert-butylcarbonylaminoethyl; wherein the aryl part of the arylmethyl is optionally substituted with one, two, three, four, or five groups independently selected from carboxyC₁-C₆alkyl, ethynylmethoxy, and hydroxy;
R¹⁴ is —C(O)NR^{14′}CR¹⁵R^{15′}R^{15″}, —C(O)NH(CH₂)_{o′}Ph(CH₂)_{o′}C(O)NHCHR¹⁷R^{17′}, —C(O)NH(CH₂)_{o′}cyclopropyl(CH₂)_{o′}C(O)NHCHR¹⁷R^{17′}, or —C(O)NR⁵⁰R⁵¹, wherein:
   R⁵⁰ and R⁵¹, together with the nitrogen atom to which they are attached, form a piperazine ring, wherein the ring is further substituted with one —(CH₂)_{o′}C(O)NHCHR¹⁷R¹⁷ group;
   o′ is 0, 1, or 2;
   R^{14′} is hydrogen or C₁-C₆alkyl, or R¹⁵ and R^{14′}, together with the atoms to which they are attached, form a morpholine, piperazine, or piperidine, ring;
   R¹⁵ is selected from hydrogen, C₂-C₆alkenyl, C₁-C₁₆alkyl, C₁-C₆alkylcarbonylaminoC₁-C₆alkyl, C₂-C₆alkynyl, aminoC₁-C₆alkyl, aminocarbonylC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, and hydroxyC₁-C₆alkyl;
   R^{15′} is hydrogen or R¹⁵ and R^{15′}, together with the atoms to which they are attached, form a C₃-C₈cycloalkyl ring; and
   R^{15″} is —(CH₂)ₘCO₂H CH₂O((CH₂)₂O)ₙCH₂C(O)NHCHR¹⁶R^{16′}, or —C(O)NHCHR¹⁶R^{16′}; wherein:
      R¹⁶ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
      R^{16′} is —(CH₂)ₘCO₂H, —CH₂O((CH₂)₂O)ₙCH₂C(O)NR⁷⁵CR^{17″}R¹⁷R^{17′}, -Ph(CH₂)_{o′}C(O)NHCHR¹⁷R^{17′} or —(CH₂)_{o′}C(O)NHCHR¹⁷R¹⁷; wherein:
         R⁷⁵ is hydrogen;
         R¹⁷ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; or R¹⁷ and R⁷⁵, together with the atoms to which they are attached, form a pyrrolidine ring;
         R^{17′} is —CH₂O((CH₂)₂O)ₙCH₂C(O)NHCHR¹⁸R^{18′}, —(CH₂)ₘCO₂H or —(CH₂)ₘC(O)NHR¹⁸R^{18′}; and
         R^{17″} is hydrogen, or R^{17″} and R¹⁷ form a C₃-C₈ cycloalkyl ring; wherein:
         R¹⁸ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R¹⁸ is —(CH₂)ₘCO₂H, —(CH₂)ₘC(O)NR¹⁹R^{19′}, or —CH₂O((CH₂)₂O)ₙCH₂C(O)NHCHR¹⁹R¹⁹; wherein:
         R¹⁹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl;
         R¹⁹ is —(CH₂)ₘC(O)NR¹⁹R^{19′}, —(CH₂)ₘCO₂H, or —CH₂O((CH₂)₂O)ₙCH₂C(O)NHCHR²⁰R^{20′}; wherein:
         R²⁰ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{20′} is —(CH₂)ₘCO₂H or —(CH₂)ₘC(O)NR²¹R^{21′}; wherein:
         R²¹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{21′} is —(CH₂)ₘCO₂H or —(CH₂)ₘC(O)NR²²R^{22′}; wherein:
         R²² is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{22′} is —(CH₂)ₘCO₂H; wherein
      m is a integer from 1 to 10;
      n is 1, 2, or 3; and
      o′ is 0, 1, or 2;
R^{a} is hydrogen or C₁-C₆alkyl;
R^{c} is hydrogen or C₁-C₆alkyl;
R^{d} is hydrogen or C₁-C₆alkyl; and
R^{e} is hydrogen or C₁-C₆alkyl.

In some aspects, the present disclosure provides a compound of formula (I), or a pharmaceutically acceptable salt thereof, wherein:
R¹ is selected from aminocarbonylmethyl, aminoethyl, aminomethyl, arylmethyl, butyl, cyclobutylmethyl, cyclohexylmethyl, cyclopropyl, cyclopropylcarbonylaminoethyl, ethyl, guanidinylbutyl, guanidinylpropyl, hydroxyethyl, hydroxymethyl, imidazolylmethyl, methoxymethyl, methyl, methylaminomethyl, morpholinylmethyl, propyl, pyridinylmethyl, thiophenylmethyl, tert-butylcarbonylaminoethyl; wherein the aryl part of the arylmethyl is optionally substituted with one, two, or three groups independently selected from aminocarbonyl, carboxy, carboxymethoxy, cyano, fluoro, and trifluoromethyl;
R² is heteroarylC₁-C₆alkyl or arylmethyl; wherein the aryl part of the arylmethyl is optionally substituted with one, two, or three groups independently selected from carboxy, carboxyC₁-C₆alkoxy, cyano, and hydroxy;
R³ is carboxymethyl;
R⁴ is selected from arylmethyl, heteroarylmethyl, and indolylmethyl; wherein the aryl part of the arylmethyl are optionally substituted with one or more groups independently selected from fluoro, methyl, hydroxy, and trifluoromethyl;
R⁵ is selected from arylmethyl, hydroxyisopropyl, isobutyl, isopentenyl, isopentenylmethyl, isopropyl, pyridinylmethyl, thiophenylmethyl, and tert-butylcarbonylaminobutyl; wherein the aryl part of the arylmethyl is optionally substituted with one, two, three, four, or five groups independently selected from amino, aminocarbonyl, carboxy, carboxymethoxy, cyano, fluoro, hydroxy, methyl, and trifluoromethyl;
R⁶ is biarylC₁-C₃alkyl;
R⁷ is selected from aminocarbonylaminopropyl, aminocarbonylethyl, arylmethyl, carboxybutyl, carboxyethyl, heteroarylC₁-C₆alkyl, isopentenyl, isopropyl, and methylcarbonylaminobutyl, wherein the aryl part of the arylmethyl is optionally substituted with one, two, three, four, or five groups independently selected from amino, aminoC₁-C₆alkyl, aminocarbonyl, carboxy, carboxyC₁-C₆alkyl, chloro, fluoro, hydroxy, and trifluoromethyl;
R⁸ is selected from aminobutyl, aminocarbonylaminopropyl, aminocarbonylethyl, aminoethyl, aminomethyl, aminopropyl, and imidazolylmethyl;
R⁹ is selected from butyl, cyclopropylmethyl, and isopropylmethyl;
R¹⁰ is selected from aminomethyl, aminoethyl and imidazolylmethyl;
R¹¹ is isobutyl, cyclobutylmethyl, cyclohexylmethyl, and arylmethyl wherein the aryl part of the arylmethyl is optionally substituted with one, two, three, four, or five groups independently selected from methyl;
R¹² is selected from aminobutyl, fluoroisopropyl, hydroxyethyl, hydroxyisopropyl, hydroxymethyl, hydroxypropyl, imidazolylmethyl, isobutyl, isopropyl, and trimethylmethyl;
R¹³ is selected from aminocarbonylaminopropyl, aminocarbonylbutyl, aminocarbonylpropyl, aminocarbonylethyl, aminobutyl, aminoethyl, aminomethyl, aminopropyl, carboxyethyl, carboxymethyl, guanidinylpropyl hydroxyethyl, hydroxymethyl, hydroxypropyl, imidazolylmethyl, methylcarboxyaminobutyl, and tert-butylcarbonylaminoethyl;
R¹⁴ is —C(O)NR^{14′}CR¹⁵R^{15′}R^{15″}, —C(O)NH(CH₂)_{o′}Ph(CH₂)_{o′}C(O)NHCHR¹⁷R¹⁷, —C(O)NH(CH₂)_{o′}cyclopropyl(CH₂)_{o′}C(O)NHCHR¹⁷R¹⁷, or —C(O)NR⁵⁰R⁵¹, wherein:
   R⁵⁰ and R⁵¹, together with the nitrogen atom to which they are attached, form a piperazine ring, wherein the ring is further substituted with one —(CH₂)_{o′}C(O)NHCHR¹⁷R^{17′} group;
   o′ is 0, 1, or 2;
   R^{14′} is hydrogen or C₁-C₆alkyl, or R¹⁵ and R^{14′}, together with the atoms to which they are attached, form a morpholine, piperazine, or piperidine, ring;
   R¹⁵ is selected from hydrogen, C₂-C₆alkenyl, C₁-C₁₆alkyl, C₁-C₆alkylcarbonylaminoC₁-C₆alkyl, C₂-C₆alkynyl, aminoC₁-C₆alkyl, aminocarbonylC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, and hydroxyC₁-C₆alkyl;
   R^{15′} is hydrogen or R¹⁵ and R^{15′}, together with the atoms to which they are attached, form a C₃-C₈cycloalkyl ring; and
   R^{15″} is —(CH₂)ₘCO₂H CH₂O((CH₂)₂O)ₙCH₂C(O)NHCHR¹⁶R^{16′}, or —C(O)NHCHR¹⁶R^{16′};
      R¹⁶ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
      R^{16′} is —(CH₂)ₘCO₂H, —CH₂O((CH₂)₂O)ₙCH₂C(O)NR⁷⁵CR^{17″}R¹⁷R^{17′}; -Ph(CH₂)_{o′}C(O)NHCHR¹⁷R^{17′} or —(CH₂)_{o′}C(O)NHCHR¹⁷R^{17′}; wherein:
         R⁷⁵ is hydrogen;
         R¹⁷ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; or R¹⁷ and R⁷⁵, together with the atoms to which they are attached, form a pyrrolidine ring;
         R^{17′} is —CH₂O((CH₂)₂O)ₙCH₂C(O)NHCHR¹⁸R^{18′}, —(CH₂)ₘCO₂H or —(CH₂)ₘC(O)NHR¹⁸R^{18′}; and
         R^{17″} is hydrogen, or R^{17″} and R¹⁷ form a C₃-C₈ cycloalkyl ring; wherein:
         R¹⁸ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R¹⁸ is —(CH₂)ₘCO₂H, —(CH₂)ₘC(O)NR¹⁹R^{19′}, or —CH₂O((CH₂)₂O)ₙCH₂C(O)NHCHR¹⁹R^{19′}; wherein:
         m is an integer between 1 and 10;
         n is 1, 2, or 3;
         R¹⁹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl;
         R¹⁹ is —(CH₂)ₘC(O)NR¹⁹R¹⁹, —(CH₂)ₘCO₂H, or —CH₂O((CH₂)₂O)ₙCH₂C(O)NHCHR²⁰R^{20′}; wherein:
         R²⁰ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{20′} is —(CH₂)ₘCO₂H or —(CH₂)ₘC(O)NR²¹R^{21′}; wherein:
         R²¹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{21′} is —(CH₂)ₘCO₂H or —(CH₂)ₘC(O)NR²²R²²; wherein:
         R²² is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{22′} is —(CH₂)ₘCO₂H; wherein:
      m is a integer from 1 to 10;
      n is 1, 2, or 3;
      o′ is 0, 1, or 2;
R^{a} is hydrogen or C₁-C₆alkyl;
R^{c} is hydrogen or C₁-C₆alkyl;
R^{d} is hydrogen or C₁-C₆alkyl; and
R^{e} is hydrogen or C₁-C₆alkyl.

In some aspects, the present disclosure provides a compound of formula (I), or a pharmaceutically acceptable salt thereof, wherein
R¹ is selected from methyl, ethyl, propyl, butyl, hydroxyethyl, aminomethyl, aminoethyl, aminocarbonylmethyl, methoxymethyl, guanidinylbutyl, guanidinylpropyl, imidazolylmethyl, pyridinylmethyl, morpholinylmethyl, cyclobutylmethyl, cyclohexylmethyl, and arylmethyl; wherein the aryl part of the arylmethyl is optionally substituted with one, two, or three groups independently selected from aminocarbonyl, carboxy, carboxymethoxy, cyano, fluoro, and trifluoromethyl;
R² is arylmethyl; wherein the aryl part of the arylmethyl is optionally substituted with one, two, or three groups independently selected from carboxy, carboxyC₁-C₆alkoxy, cyano, and hydroxy;
R³ is carboxymethyl;
R⁴ is selected from arylmethyl, heteroarylmethyl, and indolylmethyl; wherein the aryl part of the arylmethyl are optionally substituted with one or more groups independently selected from methyl and trifluoromethyl;
R⁵ is arylmethyl or isopropyl; wherein the aryl part of the arylmethyl is optionally substituted with one, two, three, four, or five groups independently selected from amino, aminocarbonyl, carboxy, carboxymethoxy, cyano, fluoro, hydroxy, methoxy, methyl, and trifluoromethyl;
R⁶ is biarylC₁-C₃alkyl;
R⁷ is selected from isobutyl, isopropyl, methylcarbonylaminobutyl, aminocarbonylethyl, aminocarbonylaminopropyl, and arylmethyl wherein the aryl part of the arylmethyl is optionally substituted with one, two, three, four, or five groups independently selected from carboxyl, carboxyalkyl, carboxymethoxy, hydroxy, and trifluoromethyl;
R⁸ is selected from aminobutyl, aminoethyl, aminopropyl, and imidazolylmethyl;
R⁹ is isopropylmethyl, and the stereochemistry of the center to which R⁹ is attached is R-stereochemistry;
R¹⁰ is aminomethyl, aminoethyl or imidazolylmethyl;
R¹¹ is C₆cycloalkylmethyl;
R¹² is selected from hydroxyethyl, hydroxyisopropyl, fluoroisopropyl, isopropyl,
R¹³ is selected from aminobutyl, aminocarbonylaminopropyl, aminocarboxyethyl, aminoethyl, aminomethyl, aminopropyl, carboxyethyl, carboxymethyl, carboxypropyl, guanidinylpropyl, hydroxyethyl, hydroxymethyl, and imidazolylmethyl;
R¹⁴ is —C(O)NR^{14′}CR¹⁵R^{15′}R^{15″}, —C(O)NH(CH₂)_{o′}Ph(CH₂) o C(O)NHCHR¹⁷R^{17′}, —C(O)NH(CH₂)_{o′}cyclopropyl(CH₂)_{o′}C(O)NHCHR¹⁷R^{17′}, or —C(O)NR⁵⁰R⁵¹, wherein:
   R⁵⁰ and R⁵¹, together with the nitrogen atom to which they are attached, form a piperazine ring, wherein the ring is further substituted with one —(CH₂)_{o′}C(O)NHCHR¹⁷R^{17′} group;
   o′ is 0, 1, or 2;
   R^{14′} is hydrogen or C₁-C₆alkyl, or R¹⁵ and R^{14′}, together with the atoms to which they are attached, form a morpholine, piperazine, or piperidine ring;
   R¹⁵ is selected from hydrogen, C₂-C₆alkenyl, C₁-C₁₆alkyl, C₁-C₆alkylcarbonylaminoC₁-C₆alkyl, C₂-C₆alkynyl, aminoC₁-C₆alkyl, aminocarbonylC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, and hydroxyC₁-C₆alkyl;
   R^{15′} is hydrogen or R¹⁵ and R^{15′}, together with the atoms to which they are attached, form a C₃-C₈cycloalkyl ring; and
   R^{15″} is —(CH₂)ₘCO₂H CH₂O((CH₂)₂O)ₙCH₂C(O)NHCHR¹⁶R^{16′}, or —C(O)NHCHR¹⁶R^{16′}, wherein:
      m is a integer from 1 to 10;
      R¹⁶ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
      R^{16′} is —(CH₂)ₘCO₂H, —CH₂O((CH₂)₂O)ₙCH₂C(O)NR⁷⁵CR^{17″}R¹⁷R^{17′}, -Ph(CH₂)_{o′}C(O)NHCHR¹⁷R^{17′} or —(CH₂)_{o′}C(O)NHCHR¹⁷R^{17′}; wherein:
         R⁷⁵ is hydrogen;
         R¹⁷ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; or R¹⁷ and R⁷⁵, together with the atoms to which they are attached, form a pyrrolidine ring;
         R^{17′} is —CH₂O((CH₂)₂O)ₙCH₂C(O)NHCHR¹⁸R^{18′}, —(CH₂)ₘCO₂H or —(CH₂)ₘC(O)NHR¹⁸R^{18′}; and
         R¹⁷″ is hydrogen, or R^{17″} and R¹⁷ form a C₃-C₈ cycloalkyl ring; wherein:
         R¹⁸ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R¹⁸ is —(CH₂)ₘCO₂H, —(CH₂)ₘC(O)NR¹⁹R^{19′}, or —CH₂O((CH₂)₂O)ₙCH₂C(O)NHCHR¹⁹R^{19′}; wherein:
         R¹⁹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl;
         R^{19′} is —(CH₂)ₘC(O)NR¹⁹R^{19′}, —(CH₂)ₘCO₂H, or —CH₂O((CH₂)₂O)ₙCH₂C(O)NHCHR²⁰R^{20′}; wherein:
         R²⁰ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{20′} is —(CH₂)ₘCO₂H or —(CH₂)ₘC(O)NR²¹R^{21′}; wherein:
         R²¹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R^{21′} is —(CH₂)ₘCO₂H or —(CH₂)ₘC(O)NR²²R^{22′}; wherein:
         R²² is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
         R²² is —(CH₂)ₘCO₂H; wherein:
      m is a integer from 1 to 10;
      n is 1, 2, or 3;
      o′ is 0, 1, or 2;
R^{a} is hydrogen;
R^{c} is hydrogen or C₁-C₆alkyl;
R^{d} is hydrogen or C₁-C₆alkyl; and
R^{e} is hydrogen or C₁-C₆alkyl.

In some aspects, the present disclosure provides a compound of formula (I), or a pharmaceutically acceptable salt thereof, wherein the compound is one of compounds 1000 to 3912 listed in Table 3.

In some aspects, the present disclosure provides a pharmaceutical composition comprising a compound of any of the above aspects, or a pharmaceutically acceptable salt thereof.

In some aspects, the present disclosure provides a method of enhancing, stimulating, and/or increasing an immune response in a subject in need thereof, wherein the method comprises administering to the subject a therapeutically effective amount of a compound of any of the above aspects, or a pharmaceutically acceptable salt thereof.

In some aspects, the present disclosure provides a method of blocking the interaction of PD-1 with PD-L1 in a subject, wherein the method comprises administering to the subject a therapeutically effective amount of a compound of any of the above aspects, or a pharmaceutically acceptable salt thereof.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of formula (I) or a pharmaceutically acceptable salt thereof” includes, but is not limited to, a compound of formula (I), two compounds of formula (I), a pharmaceutically acceptable salt of a compound of formula (I), a compound of formula (I) and one or more pharmaceutically acceptable salts of the compound of formula (I), and two or more pharmaceutically acceptable salts of a compound of formula (I).

The term “C₂-C₆alkenyl,” as used herein, refers to a group derived from a straight or branched chain hydrocarbon containing one or more carbon-carbon double bonds containing two to six carbon atoms.

The term “C₁-C₆alkoxy”, as used herein, refers to a C₁-C₆alkyl group attached to the parent molecular moiety through an oxygen atom.

The term “alkyl,” as used herein, refers to a group derived from a straight or branched chain saturated hydrocarbon containing carbon atoms. The term “alkyl” may be proceeded by “C_{#}-C_{#}” wherein the # is an integer and refers to the number of carbon atoms. For example, C₁-C₂alkyl contains one to two carbon atoms and C₁-C₃alkyl contains one to three carbon atoms.

The term “C₁-C₂alkylamino,” as used herein, refers to a group having the formula —NHR, wherein R is a C₁-C₂alkyl group.

The term “C₁-C₂alkylaminoC₁-C₆alkyl,” as used herein, refers to a C₁-C₂alkylamino group attached to the parent molecular moiety through a C₁-C₆alkyl group.

The term “C₁-C₆alkylcarbonyl,” as used herein, refers to a C₁-C₆alkyl group attached to the parent molecular moiety through a carbonyl group.

The term “C₁-C₂alkylcarbonylamino,” as used herein, refers to —NHC(O)R^{a}, wherein R^{a} is a C₁-C₆alkyl group.

The term “C₁-C₆alkylcarbonylamino,” as used herein, refers to —NHC(O)R^{a}, wherein R^{a} is a C₁-C₂alkyl group.

The term “C₁-C₂alkylcarbonylaminoC₁-C₆alkyl,” as used herein, refers to a C₁-C₂alkylcarbonylamino group attached to the parent molecular moiety through a C₁-C₆alkyl group.

The term “C₁-C₆alkylcarbonylaminoC₁-C₆alkyl,” as used herein, refers to a C₁-C₆alkylcarbonylamino group attached to the parent molecular moiety through a C₁-C₆alkyl group.

The term “C₁-C₆alkylheteroaryl,” as used herein, refers to a heteroaryl group substituted with one, two, or three C₁-C₆alkyl groups.

The term “C₁-C₆alkylheteroarylC₁-C₆alkyl,” as used herein, refers to a C₁-C₆alkylheteroaryl group attached to the parent molecular moiety through a C₁-C₆alkyl group.

The term “C₁-C₆alkylimidazolyl,” as used herein, refers to an imiadazolyl ring substituted with one, two, or three C₁-C₆alkyl groups.

The term “C₁-C₆alkylimidazolylC₁-C₂alkyl,” as used herein, refers to a C₁-C₆alkylimidazolyl group attached to the parent molecular moiety through a C₁-C₂alkyl group.

The term “C₂-C₆alkynyl,” as used herein, refers to a group derived from a straight or branched chain hydrocarbon containing one or more carbon-carbon triple bonds containing two to six carbon atoms.

The term “C₂-C₆alkynylmethoxy,” as used herein, refers to a C₂-C₆alkynylmethyl group attached to the parent molecular moiety through an oxygen atom.

The term “C₂-C₆alkynylmethyl,” as used herein, refers to a C₂-C₆alkynyl group attached to the parent molecular moiety through a CH₂ group.

The term “amino,” as used herein, refers to —NH₂.

The term “aminoC₁-C₃alkyl,” as used herein, refers to an amino group attached to the parent molecular moiety through a C₁-C₃alkyl group.

The term “aminoC₁-C₆alkyl,” as used herein, refers to an amino group attached to the parent molecular moiety through a C₁-C₆alkyl group.

The term “aminobutyl,” as used herein, refers to —CH₂CH₂CH₂CH₂NH₂.

The term “aminocarbonyl,” as used herein, refers to an amino group attached to the parent molecular moiety through a carbonyl group.

The term “aminocarbonylC₁-C₂alkyl,” as used herein, refers to an aminocarbonyl group attached to the parent molecular moiety through a C₁-C₂alkyl group.

The term “aminocarbonylC₁-C₃alkyl,” as used herein, refers to an aminocarbonyl group attached to the parent molecular moiety through a C₁-C₃alkyl group.

The term “aminocarbonylC₁-C₆alkyl,” as used herein, refers to an aminocarbonyl group attached to the parent molecular moiety through a C₁-C₆alkyl group.

The term “aminocarbonylamino,” as used herein, refers to an aminocarbonyl group attached to the parent molecular moiety through an amino group.

The term “aminocarbonylaminoC₁-C₆alkyl,” as used herein, refers to an aminocarbonylamino group attached to the parent molecular moiety through a C₁-C₆alkyl group.

The term “aminocarbonylaminoC₂-C₆alkyl,” as used herein, refers to an aminocarbonylamino group attached to the parent molecular moiety through a C₂-C₆alkyl group.

The term “aminocarbonylaminomethyl,” as used herein, refers to an aminocarbonylamino group attached to the parent molecular moiety through a CH₂ group.

The term “aminocarbonylaminopropyl,” as used herein, refers to an aminocarbonylamino group attached to the parent molecular moiety through a CH₂CH₂CH₂ group.

The term “aminocarbonylmethyl,” as used herein, refers to an aminocarbonyl group attached to the parent molecular moiety through a CH₂ group.

The term “aminoethyl,” as used herein, refers to —CH₂CH₂NH₂.

The term “aminomethyl,” as used herein, refers to —CH₂NH₂.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₂alkyl,” as used herein, refers to an aryl group attached to the parent molecular moiety through a C₁-C₂alkyl group.

The term “arylmethyl,” as used herein, refers to an aryl group attached to the parent molecular moiety through a CH₂ group.

The term “biaryl,” as used herein, refers to an aryl group substituted with one additional aryl group.

The term “biarylC₁-C₆alkyl,” as used herein, refers to a biaryl group attached to the parent molecular moiety through a C₁-C₆alkyl group.

The term “carbonyl,” as used herein, refers to —C(O)—.

The term “carboxy”, as used herein, refers to —CO₂H.

The term “carboxyC₁-C₆alkoxy,” as used herein, refers to a carboxyC₁-C₆alkyl group attached to the parent molecular moiety through an oxygen atom.

The term “carboxyC₁-C₆alkyl”, as used herein, refers to a carboxy group attached to the parent molecular moiety through a C₁-C₆alkyl group.

The term “carboxymethoxy,” as used herein, refers to —OCH₂CO₂H.

The term “carboxymethy,” as used herein, refers to —CH₂CO₂H.

The term “cyano,” as used herein, refers to —CN.

The term “cyanoC₁-C₆alkyl,” as used herein, refers to a cyano group attached to the parent molecular moiety though a C₁-C₆alkyl.

The term “C₃-C₆cycloalkyl”, as used herein, refers to a saturated monocyclic or bicyclic hydrocarbon ring system having three to six carbon atoms and zero heteroatoms. The bicyclic rings can be fused, spirocyclic, or bridged. Representative examples of cycloalkyl groups include, but are not limited to, cyclopropyl, cyclopentyl, and cyclohexyl.

The term “C₃-C₈cycloalkyl”, as used herein, refers to a saturated monocyclic or bicyclic hydrocarbon ring system having three to eight carbon atoms and zero heteroatoms. The bicyclic rings can be fused, spirocyclic, or bridged. Representative examples of cycloalkyl groups include, but are not limited to, cyclopropyl, cyclopentyl, cyclohexyl, cycloheptyl, and cyclooctyl.

The term “(C₃-C₆cycloalkyl) C₁-C₂alkyl”, as used herein, refers to a C₃-C₆cycloalkyl group attached to the parent molecular moiety through a C₁-C₂alkyl group.

The term “(C₃-C₆cycloalkyl) C₁-C₆alkyl”, as used herein, refers to a C₃-C₆cycloalkyl group attached to the parent molecular moiety through a C₁-C₆alkyl group.

The term “C₃-C₆cycloalkylcarbonyl,” as used herein, refers to a C₃-C₆cycloalkyl group attached to the parent molecular moiety through a carbonyl group.

The term “C₃-C₆cycloalkylcarbonylamino,” as used herein, refers to a C₃-C₆cycloalkylcarbonyl group attached to the parent molecular moiety through an amino group.

The term “C₃-C₆cycloalkylcarbonylaminoC₁-C₆alkyl,” as used herein, refers to a C₃-C₆cycloalkylcarbonylamino group attached to the parent molecular moiety through a C₁-C₆alkyl group.

The term “(C₃-C₆cycloalkyl)methyl”, as used herein, refers to a C₃-C₆cycloalkyl group attached to the parent molecular moiety through a CH₂ group.

The term “cyclopropylcarbonylaminoethyl,” as used herein, refers to —CH₂CH₂NHC(O)R, wherein R is a cyclopropyl group.

The term “difluorocyclohexylmethyl,” as used herein refers to a cyclohexyl group substituted with two fluoro groups that is attached to the parent molecular moiety through a CH₂ group.

The term “ethynylmethoxy,” as used herein, refers to —OCH₂C≡CH.

The term “fluoroC₁-C₆alkyl,” as used herein, refers to a C₁-C₆alkyl group substituted by one, two, three, or four fluoro groups.

The term “fluoroC₁-C₆alkylcarbonyl,” as used herein, refers to a fluoroC₁-C₆alkyl group attached to the parent molecular moiety through a carbonyl group.

The term “fluoroC₁-C₆alkylcarbonylamino,” as used herein, refers to a fluoroC₁-C₆alkylcarbonyl group attached to the parent molecular moiety through an NH group.

The term “fluoroC₁-C₆alkylcarbonylaminoC₁-C₆alkyl,” as used herein, refers to a fluoroC₁-C₆alkylcarbonylamino group attached to the parent molecular moiety through a C₁-C₆alkyl group.

The term “fluoroC₄-C₆alkyl,” as used herein, refers to a C₄-C₆alkyl group substituted by one, two, three, or four fluoro groups.

The term “fluoroheterocyclyl,” as used herein, refers to a heterocyclyl group substituted with one, two, or three fluoro groups.

The term “fluoroheterocyclylC₁-C₆alkyl,” as used herein, refers to a fluoroheterocyclyl group attached to the parent molecular moiety through a C₁-C₆alkyl group.

The term “guanidinylC₁-C₆alkyl,” as used herein, refers to a NH₂C(NH)NH— group attached to the parent molecular moiety through a C₁-C₆alkyl group.

The term “guanidinylC₂-C₄alkyl,” as used herein, refers to a NH₂C(NH)NH— group attached to the parent molecular moiety through a C₂-C₄alkyl group.

The term “guanidinylC₂-C₆alkyl,” as used herein, refers to a NH₂C(NH)NH— group attached to the parent molecular moiety through a C₂-C₆alkyl group.

The terms “halo” and “halogen”, as used herein, refer to F, Cl, Br, or I.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d′]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl,” as used herein, refers to a heteroaryl group attached to the parent molecular moiety through a C₁-C₆alkyl group.

The term “heteroarylmethyl,” as used herein, refers to a heteroaryl group attached to the parent molecular moiety through a CH₂ group.

The term “heterocyclyl,” as used herein, refers to a five-, six-, or seven-membered non-aromatic ring containing one, two, or three heteroatoms independently selected from nitrogen, oxygen, and sulfur. The term “heterocyclyl” also includes bicyclic groups in which the heterocyclyl ring is fused to a four- to six-membered aromatic or non-aromatic carbocyclic ring or another monocyclic heterocyclyl group. The heterocyclyl groups of the present disclosure can be attached to the parent molecular moiety through any substitutable atom in the group. Examples of heterocyclyl groups include, but are not limited to, morpholinyl, piperazinyl, pyrrolidinyl, and thiomorpholinyl.

The term “heterocyclylC₁-C₆alkyl,” as used herein, refers to a heterocyclyl attached to the parent molecular moiety through a C₁-C₆alkyl group.

The term “hydroxy,” as used herein, refers to —OH.

The term “hydroxyC₁-C₃alkyl,” as used herein, refers to a hydroxy group attached to the parent molecular moiety through a C₁-C₃alkyl group.

The term “hydroxyC₁-C₆alkyl,” as used herein, refers to a hydroxy group attached to the parent molecular moiety through a C₁-C₆alkyl group.

The term “hydroxyaryl,” as used herein, refers to an aryl group substituted with one, two, or three hydroxy groups.

The term “hydroxyarylC₁-C₂alkyl,” as used herein, refers to a hydroxyaryl group attached to the parent molecular moiety through a C₁-C₂alkyl group.

The term “indolylC₁-C₆alkyl,” as used herein, refers to an indolyl group attached to the parent molecular moiety through a C₁-C₆alkyl group.

The term “methoxy,” as used herein, refers to —OCH₃.

The term “methoxyC₁-C₂alkyl,” as used herein, refers to a methoxy group attached to the parent molecular moiety though a C₁-C₂alkyl group.

The term “methylcarbonylamino,” as used herein, refers to —NHC(O)CH₃.

The term “methylcarbonylaminobutyl,” as used herein, refers to —(CH₂)₄NHC(O)CH₃.

The term “methylcarbonylaminobutyl,” as used herein, refers to —(CH₂)₃NHC(O)CH₃.

The term “methylsulfanyl,” as used herein, refers to a —S—CH₃.

The term “methylsulfanylC₁-C₆alkyl,” as used herein, refers to a methylsulfanyl group attached to the parent molecular moiety through a C₁-C₆alkyl group.

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Programmed Death Ligand 1”, “Programmed Cell Death Ligand 1”, “PD-L1”, “PDL1”, “hPD-L1”, “hPD-L1”, and “B7-H1” are used interchangeably, and include variants, isoforms, species homologs of human PD-L1, and analogs having at least one common epitope with PD-L1. The complete PD-L1 sequence can be found under GENBANK® Accession No. NP_054862.

The terms “Programmed Death 1”, “Programmed Cell Death 1”, “Protein PD-1”, “PD-1”, “PD1”, “hPD-1” and “hPD-I” are used interchangeably, and include variants, isoforms, species homologs of human PD-1, and analogs having at least one common epitope with PD-1. The complete PD-1 sequence can be found under GENBANK® Accession No. U64863.

The term “treating” refers to i) inhibiting the disease, disorder, or condition, i.e., arresting its development; and/or ii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

Those of ordinary skill in the art are aware that an amino acid includes a compound represented by the general structure:

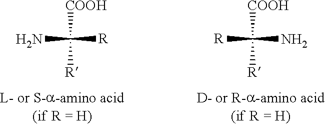

where R and R′ are as discussed herein. Unless otherwise indicated, the term “amino acid” as employed herein, alone or as part of another group, includes, without limitation, an amino group and a carboxyl group linked to the same carbon, referred to as “a” carbon, where R and/or R′ can be a natural or an un-natural side chain, including hydrogen. The absolute “S” configuration at the “a” carbon is commonly referred to as the “L” or “natural” configuration. In the case where both the “R” and the “R” (prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which possess the ability to inhibit the interaction between PD-1 and PD-L1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Certain compounds of the present disclosure can exist as tautomers, which are compounds produced by the phenomenon where a proton of a molecule shifts to a different atom within that molecule. The term “tautomer” also refers to one of two or more structural isomers that exist in equilibrium and are readily converted from one isomer to another. All tautomers of the compounds described herein are included within the present disclosure.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Administration of a therapeutic agent described herein includes, without limitation, administration of a therapeutically effective amount of therapeutic agent. The term “therapeutically effective amount” as used herein refers, without limitation, to an amount of a therapeutic agent to treat a condition treatable by administration of a composition comprising the PD-1/PD-L1 binding inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the macrocyclic peptides described herein, the dosage ranges from about 0.0001 to 100 mg/kg, and more usually 0.01 to 40 mg/kg, of the host body weight. For example dosages can be 0.3 mg/kg body weight, 1 mg/kg body weight, 3 mg/kg body weight, 5 mg/kg body weight, 10 mg/kg body weight, 20 mg/kg body weight, 30 mg/kg body weight, 40 mg/kg body weight, or within the range of 10-40 mg/kg. An exemplary treatment regime entails administration once per day, bi-weekly, tri-weekly, weekly, once every two weeks, once every three weeks, once every four weeks, once a month, once every 3 months or once every three to 6 months. Preferred dosage regimens for a macrocyclic peptide of the disclosure include 1 mg/kg body weight or 3 mg/kg body weight via intravenous administration, with the macrocyclic peptide being given using one of the following dosing schedules: (i) every four weeks for six dosages, then every three months; (ii) every three weeks; (iii) 3 mg/kg body weight once followed by 1 mg/kg body weight every three weeks.

In another aspect, the disclosure pertains to methods of inhibiting growth of tumor cells in a subject using the macrocyclic compounds of the present disclosure. In certain embodiments, the compounds of the present disclosure are capable of binding to PD-1, disrupting the interaction between PD-1 and PD-L1, competing with the binding of PD-1 with certain anti-PD-1 monoclonal antibodies that are known to block the interaction with PD-L1, and enhancing CMV-specific T cell IFNγ secretion. As a result, the compounds of the present disclosure can be useful for modifying an immune response, treating diseases such as cancer, stimulating a protective autoimmune response, or to stimulate antigen-specific immune responses (e.g., by co-administration of PD-L1 blocking compounds with an antigen of interest). For example, the compounds of the present disclosure can be used to treat cancers selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

Compounds of the present disclosure can also be used in treating infectious diseases, such as those caused by a virus. Examples of such viruses include, but are not limited to, HIV, Hepatitis A, Hepatitis B, Hepatitis C, herpes viruses, and influenza.

Compounds of the present disclosure can also be used in treating septic shock.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents. For example, the combination therapy can include a macrocyclic compound combined with at least one other anti-inflammatory or immunosuppressant agent. Examples of therapeutic agents that can be used in combination therapy are described in greater detail below in the section on uses of the compounds of the disclosure.

As used herein, “pharmaceutically acceptable carrier” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

A pharmaceutical composition of the disclosure also can include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure can be administered via one or more routes of administration using one or more of a variety of methods known in the art. As will be appreciated by the skilled artisan, the route and/or mode of administration will vary depending upon the desired results. In some embodiments, the routes of administration for macrocyclic compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of formula (I) and/or at least one salt thereof with at least one inert solid diluent, such as, for example, calcium carbonate; calcium phosphate; and kaolin.

Soft gelatin capsules can, for example, be prepared by mixing at least one compound of formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of formula (I) and/or at least one pharmaceutically acceptable salt thereof with at least one excipient suitable for the manufacture of an aqueous suspension, including, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

An emulsion of at least one compound of formula (I) and/or at least one pharmaceutically acceptable salt thereof can, for example, be prepared as an oil-in-water emulsion. The oily phase of the emulsions comprising the compounds of formula (I) can be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase can comprise merely an emulsifier, it can comprise a mixture of at least one emulsifier with a fat or an oil or with both a fat and an oil. Suitable emulsifying agents include, but are not limited to, for example, naturally-occurring phosphatides, e.g., soy bean lecithin, esters or partial esters derived from fatty acids and hexitol anhydrides, such as, for example sorbitan monoleate, and condensation products of partial esters with ethylene oxide, such as, for example, polyoxyethylene sorbitan monooleate. In some embodiments, a hydrophilic emulsifier is included together with a lipophilic emulsifier which acts as a stabilizer. It is also sometimes desirable to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceral disterate alone or with a wax, or other materials well known in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one embodiment, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the disclosure can be formulated to ensure proper distribution in vivo. For example, the blood-brain barrier (BBB) excludes many highly hydrophilic compounds. To ensure that therapeutic compounds of the disclosure cross the BBB (if desired), they can be formulated, for example, in liposomes. For methods of manufacturing liposomes, see, e.g., U.S. Pat. Nos. 4,522,811, 5,374,548, and 5,399,331. The liposomes can comprise one or more moieties which are selectively transported into specific cells or organs, thus enhance targeted drug delivery (see, e.g., Ranade, V. V., J. Clin. Pharmacol., 29:685 (1989)). Exemplary targeting moieties include folate or biotin (see, e.g., U.S. Pat. No. 5,416,016 to Low et al.); mannosides (Umezawa et al., Biochem. Biophys. Res. Commun., 153:1038 (1988)); macrocyclic compounds (Bloeman, P. G. et al., FEBS Lett., 357:140 (1995); Owais, M. et al., Antimicrob. Agents Chemother., 39:180 (1995)); surfactant protein A receptor (Briscoe et al., Am. J. Physiol., 1233:134 (1995)); p 120 (Schreier et al., J. Biol. Chem., 269:9090 (1994)); see also Keinanen, K. et al., FEBS Lett., 346:123 (1994); Killion, J. J. et al., Immunomethods 4:273 (1994).

In certain embodiments, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some embodiments, the compounds of the present disclosure can be administered orally, i.e, via a gelatin capsule, tablet, hard or soft capsule, or a liquid capsule. The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### EXAMPLES

The following Examples are included to demonstrate various aspects of the present disclosure. It should be appreciated by those of skill in the art that the techniques disclosed in the Examples that follow represent techniques discovered by the inventors to function well in the practice of the disclosure, and thus can be considered to constitute preferred modes for its practice. However, those of skill in the art should, in light of the present disclosure, appreciate that many changes can be made in the specific Compounds which are disclosed and still obtain a like or similar result without departing from the spirit and scope of the disclosure.

The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

Abbreviations used in the schemes generally follow conventions used in the art. Chemical abbreviations used in the specification and Compounds are defined as follows: Ph=phenyl; Bn=benzyl; i-Bu=iso-butyl; i-Pr=iso-propyl; Me=methyl; Et=ethyl; Pr=n-propyl; Bu=n-butyl; t-Bu=tert-butyl; Trt=trityl; TMS=trimethylsilyl; TIS=triisopropylsilane; Et₂O=diethyl ether; HOAc or AcOH=acetic acid; MeCN or AcCN=acetonitrile; DMF=N,N-dimethylformamide; EtOAc=ethyl acetate; THF=tetrahydrofuran; TFA=trifluoroacetic acid; TFE=α,α,α-trifluoroethanol; Et₂NH=diethylamine; NMM=N-methylmorpholine; NMP=N-methylpyrrolidone; DCM=dichloromethane; TEA=trimethylamine; min.=minute(s); h or hr=hour(s); L=liter; mL or ml=milliliter; μL=microliter; g=gram(s); mg=milligram(s); mol=mole(s); mmol=millimole(s); meq=milliequivalent; rt or RT=room temperature; sat or sat′d=saturated; aq.=aqueous; mp=melting point; BOP reagent=benzotriazol-1-yloxy-tris-dimethylamino-phosphonium hexafluorophosphate (Castro's reagent); PyBOP reagent=benzotriazol-1-yloxy-tripyrrolidino phosphonium hexafluorophosphate; HBTU=2-(1H-Benzotriazol-1-yl)-1,1,3,3-tetramethyluronim hexafluorophosphate; HATU=O-(7-Azabenzotriazol-1-yl)-1,1,3,3-tetramethyluronim hexafluorophosphate; HCTU=2-(6-Chloro-1-H-benzotriazol-1-yl)-1,1,3,3-tetramethyluronium hexafluorophosphate; T3P=2,4,6-tripropyl-1,3,5,2,4,6-trioxatriphosphorinane-2,4,6-trioxide; DMAP=4-(dimethylamino)pyridine; DIEA=diisopropylethylamine; Fmoc or FMOC=fluorenylmethyloxycarbonyl; Boc or BOC=tert-butyloxycarbonyl; HOBT or HOBT·H₂O=1-hydroxybenzotriazole hydrate; Cl-HOBt=6-Chloro-benzotriazole; HOAT=1-hydroxy-7-azabenzotriazole; HPLC=high performance liquid chromatography; LC/MS=high performance liquid chromatography/mass spectrometry; MS or Mass Spec=mass spectrometry; NMR=nuclear magnetic resonance; Sc or SC or SQ=sub-cutaneous; and IP or ip=intra-peritoneal.

### Example 1: General Synthetic Procedures and Analytical Methods

The macrocyclic compounds of the present disclosure can be produced by methods known in the art, such as they can be synthesized chemically, recombinantly in a cell free system, recombinantly within a cell or can be isolated from a biological source. Chemical synthesis of a macrocyclic compound of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic compounds and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The compounds can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-Cl-HOBt or HOAt active esters produced from DIC/HOBt, HBTU/HOBt, BOP, PyBOP, or from DIC/6-Cl-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the compound analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown in Table 1.

TABLE 1
Compounds of Orthogonally Protected Amino Acids used in Solid Phase Synthesis

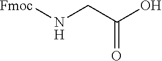

Fmoc-Gly

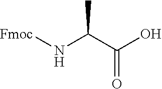

Fmoc-Ala

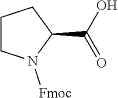

Fmoc-Pro

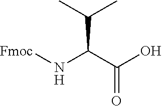

Fmoc-Val

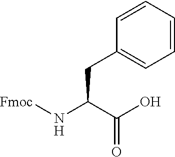

Fmoc-Phe

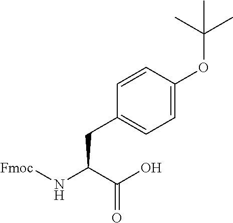

Fmoc-Tyr(tBu)

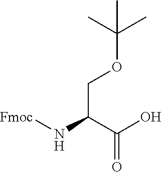

Fmoc-Ser(tBu)

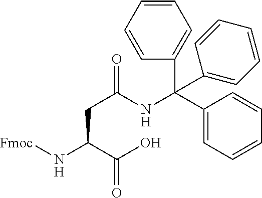

Fmoc-Asn(Trt)

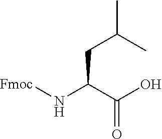

Fmoc-Leu

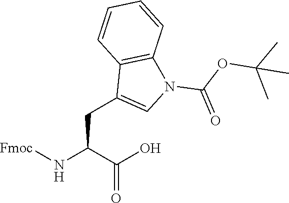

Fmoc-Trp(Boc)

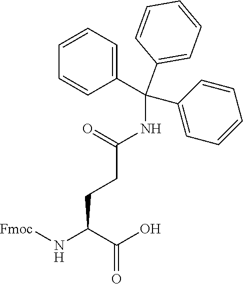

Fmoc-Gln(Trt)

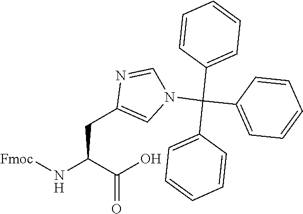

Fmoc-His(Trt)

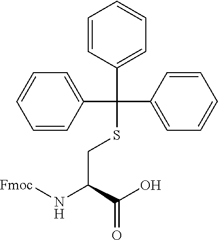

Fmoc-Cys(Trt)

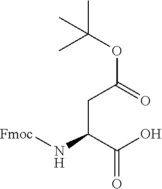

Fmoc-Asp(OBut)

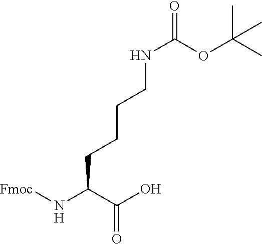

Fmoc-Lys(Boc)

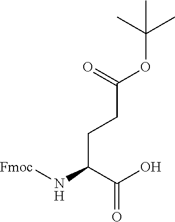

Fmoc-Glu(OBut)

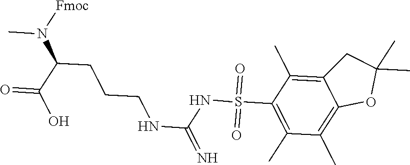

Fmoc-Arg(Pbf)

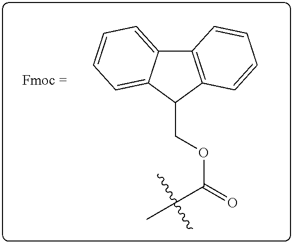 [SMILES: *C(=O)OCC1c2ccccc2-c2ccccc21]

The peptidyl-resin precursors for their respective compounds may be cleaved and deprotected using any standard procedure (see, for Compound, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 hrs at room temperature. The spent resin is then filtered off and the TFA solution was cooled and Et₂O solution was added. The precipitates were collected by centrifuging and decanting the ether layer (3×). The resulting crude peptide is either redissolved directly into DMF or DMSO or CH₃CN/H₂O for purification by preparative HPLC or used directly in the next step.

Compounds with the desired purity can be obtained by purification using preparative HPLC, for Compound, on a Waters Model 4000 or a Shimadzu Model LC-8A liquid chromatography. The solution of crude compound is injected into a YMC S5 ODS (20×100 mm) column and eluted with a linear gradient of MeCN in water, both buffered with 0.1% TFA, using a flow rate of 14-20 mL/min with effluent monitoring by UV absorbance at 217 or 220 nm. The structures of the purified compounds can be confirmed by electro-spray MS analysis.

List of unnatural amino acids referred to herein is provided in Table 2.

TABLE 2
Compounds of Orthogonally Protected Amino Acids used in Solid Phase Synthesis

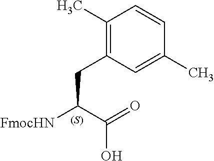

Fmoc-Phe((2,5-di-Me)-OH

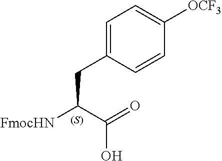

Fmoc-Phe(4-OCF_{Fmoc})-OH

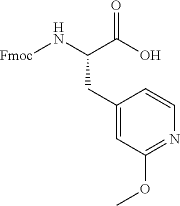

Fmoc-4-Pya(2-OMe)-OH

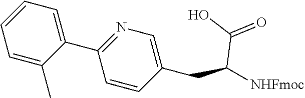

Fmoc-3-Pya(6-o-tolyl)-OH
(S)-2-((((9H-fluoren-9-
yl)methoxy)carbonyl)amino)-3-(6-
(o-tolyl)pyridin-3-yl)propanoic acid

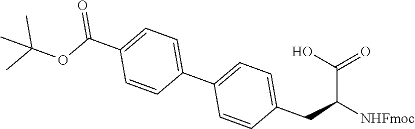

Fmoc-Bip(4′-COOtBu)-OH

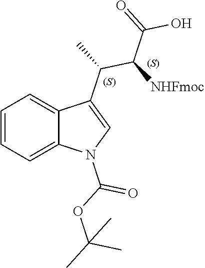

Fmoc-(S)-β-Me-Trp(Boc)-OH

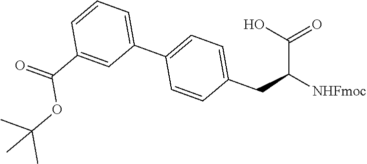

Fmoc-Bip(3′-COOtBu)-OH

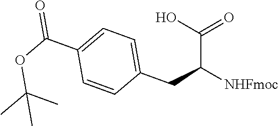

Fmoc-Phe(4-COOtBu)

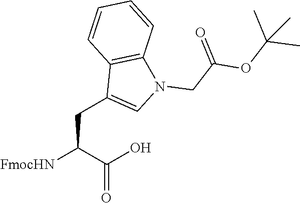

Fmoc-Trp(CH₂COOtBu)-OH

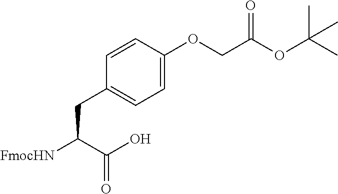

Fmoc-Tyr(CH₂COOtBu)-OH

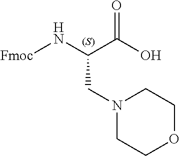

Fmoc-Ala(β-morpholin-4-yl)-OH
(2S)-2-({[(9H-fluoren-9-
yl)methoxy]carbonyl}amino)-3-
morpholin-4-yl)propanoic acid

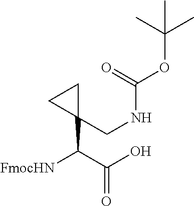

(S)-2-Fmoc-amino-2-(1-Boc-
aminomethyl-cyclopropyl)acetic acid

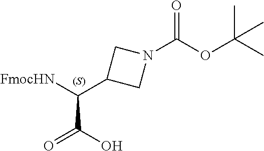

(S)-2-Fmoc-amino-2-(1-Boc-azetidin-3-
yl)acetic acid

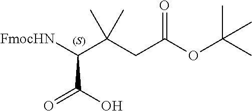

Fmoc-β,β-diMe-Glu(tBu)-OH

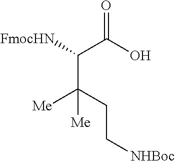

Fmoc-β,β-diMe-Orn(Boc)-OH

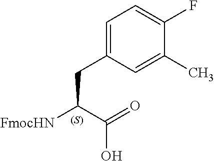

Fmoc-Phe(3-Me-4-F)-OH

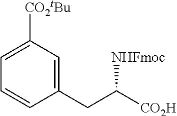

Fmoc-Phe(3-COOtBu)-OH

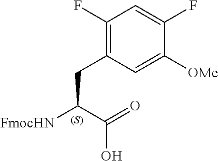

Fmoc-Phe(2,3-di-F-4-OMe)-OH

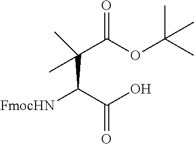

Fmoc-β,β-DiMe-Asp(tBu)-OH

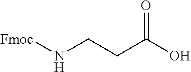

Fmoc-β-Ala-OH

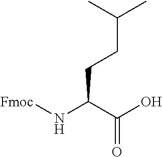

Fmoc-HomoLeu-OH

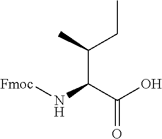

Fmoc-Ile-OH

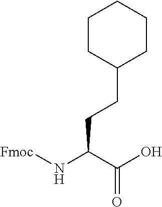

Fmoc-HomoCha-OH

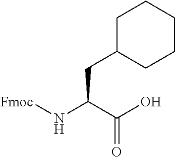

Fmoc-Cha-OH

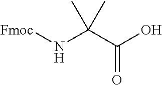

(Fmoc-Aib-OH)

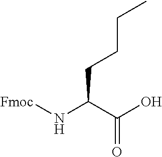

Fmoc-Nle-OH

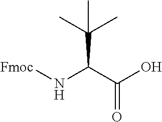

Fmoc-tBu-Gly-OH
(Fmoc-Tle-OH)

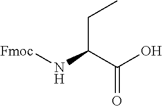

Fmoc-Abu-OH

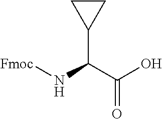

Fmoc-Cyclopropyl-Gly-OH

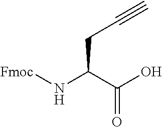

(Fmoc-Pra-OH)

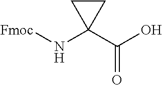

1-(Fmoc-
amino)cyclopropane
carboxylic acid

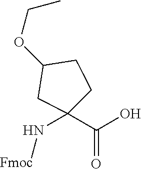

Fmoc-1-amino-3-OtBu-
cyclopentane-1-carboxylic acid

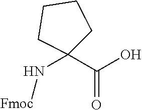

Fmoc-1-aminocyclopentane-
1-carboxylic acid

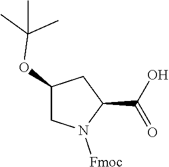

Fmoc-cis-Hyp(tBu)-OH

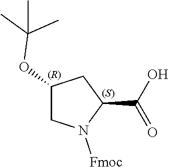

Fmoc-Hyp(tBu)-OH

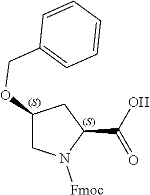

Fmoc-cis-Hyp(Bn)-OH

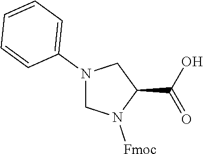

Fmoc-NPh-AzaPro-OH

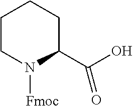

Fmoc-Pip-OH

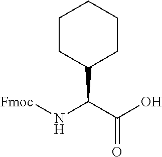

Fmoc-Chg-OH

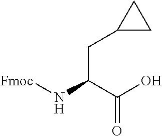

Fmoc-Ala(β-cyclopropyl)-OH
Fmoc-Cyclopropyl-Ala-OH

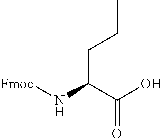

Fmoc-Nva-OH

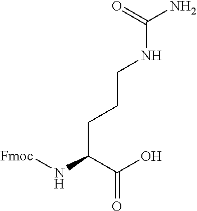

Fmoc-Cit-OH

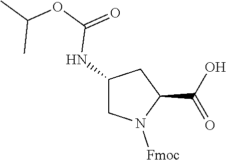

Fmoc-trans-4-NHBoc-Pro-OH

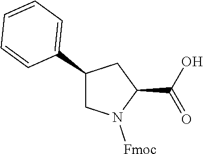

Fmoc-cis-4-Ph-Pro-OH

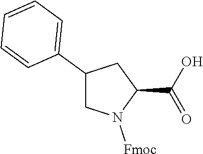

Fmoc-4-Ph-Pro-OH

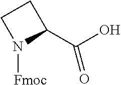

Fmoc-azetidine-2-carboxylic acid
Fmoc-Azt-OH

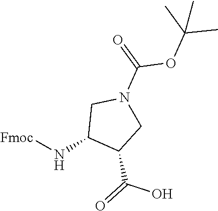

Fmoc-isoPro(Boc)-OH

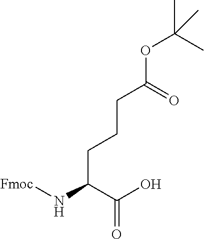

Fmoc-homoGlu(OtBu)-OH

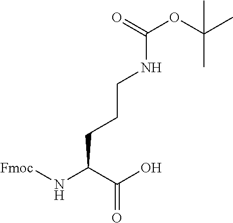

Fmoc-Orn(Boc)-OH

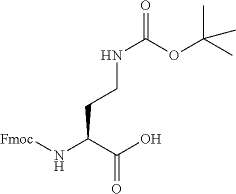

Fmoc-Dab(Boc)-OH

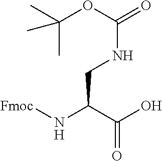

Fmoc-Dap(Boc)-OH

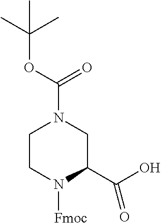

1-Fmoc-4-(tert-butoxycarbonyl)
piperazine-2-carboxylic acid
1-Fmoc-4-Boc-piperazine-OH

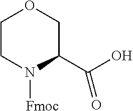

4-N-Fmoc-morpholinecarboxylic acid

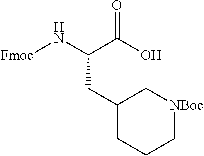

Fmoc-Ala(β-1-Boc-piperidin-3-yl)-OH

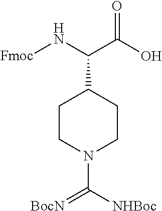

(2S)-2-{1-[(1Z)-{[(tert-
butoxy)carbonyl]amino}{{[(tert-
butoxy)carbonyl]imino})methyl]
piperidin-4-yl}-2-
({[(9H-fluoren-9-yl)methoxy]
carbonyl}amino)acetic acid

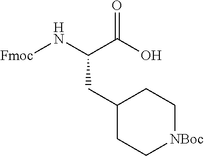

Fmoc-Ala(β-1-Boc-piperidin-4-yl)-OH

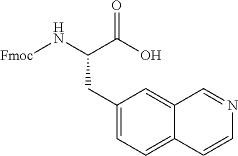

Fmoc-Ala(β-isoquinolin-7-yl)-OH

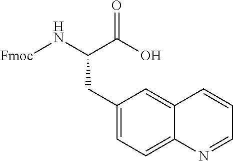

Fmoc-Ala(β-quinolin-6-yl)-OH

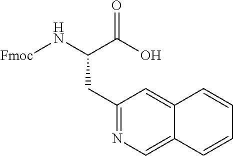

Fmoc-Ala(β-isoquinolin-3-yl)-OH

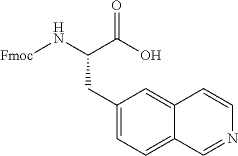

Fmo-Ala(β-
isoquinolin-6-yl)-OH

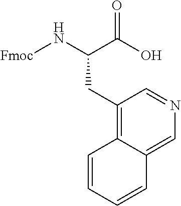

Fmoc-Ala(β-isoquinolin-4-yl)-OH

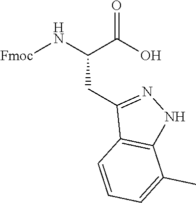

W(1,2-N2,7-Me)

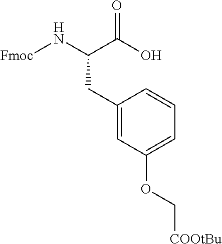

Fmoc-Phe(3-OCH₂COOtBu)-OH

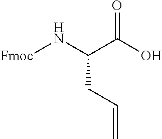

Fmoc-AllylGly-OH

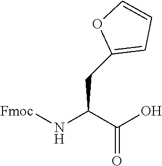

Fmoc-2-furanyl-Ala-OH

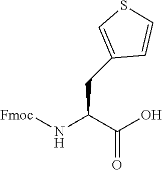

Fmoc-3-thienyl-Ala-OH

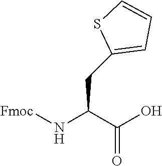

Fmoc-2-thienyl-Ala-OH

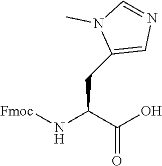

Fmoc-His(3-Me)-OH

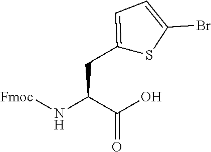

Fmoc-2-thienyl-5-Br-Ala-OH

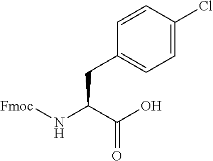

Fmoc-Phe(4-Cl)-OH

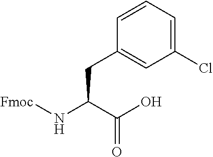

Fmoc-Phe(3-Cl)-OH

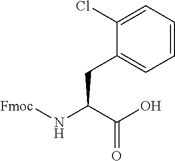

Fmoc-Phe(2-Cl)-OH

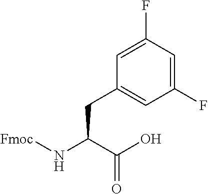

Fmoc-Phe(3,5-di-F)-OH

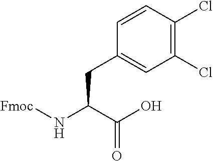

Fmoc-Phe(3,4-di-Cl)-OH

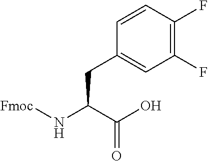

Fmoc-Phe(3,4-diF)-OH

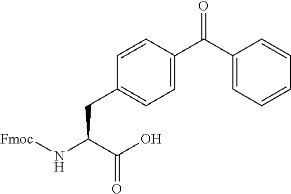

Fmoc-Bpa-OH

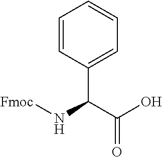

Fmoc-PhenylGly-OH
(Fmoc-Phg-OH)

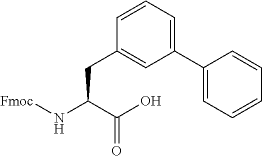

Fmoc-3-Ph-Phe-OH

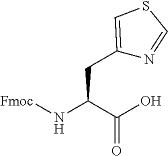

Fmoc-4-Thiazolylalanine
Fmoc-Ala(4-Thiazol-3-yl)

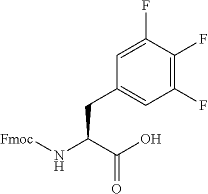

Fmoc-Phe(3,4,5-tri-F)-OH

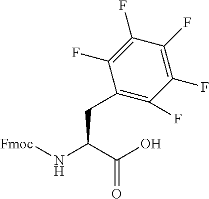

Fmoc-Phe(2,3,4,5,6-penta-F)-OH

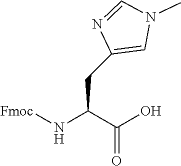

Fmoc-His(1-Me)-OH

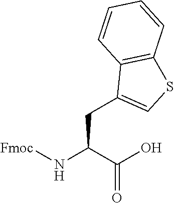

Fmoc-Bzt-OH

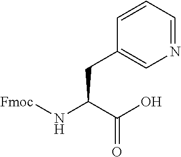

Fmoc-3-Py-Ala-OH
(Fmoc-3-Pya-OH)

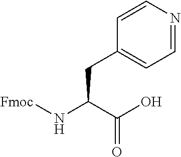

Fmoc-4-Py-Ala-OH
(Fmoc-4-Pya-OH)

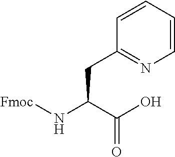

Fmoc-2-Py-Ala-OH
(Fmoc-2-Pya-OH)

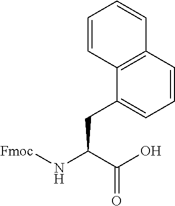

Fmoc-1-Nal-OH

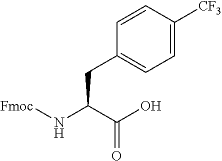

Fmoc-Phe(4-CF₃)-OH

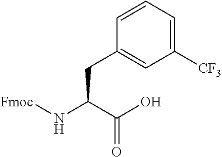

Fmoc-Phe(3-CF₃)OH

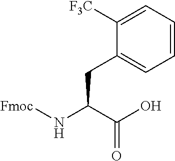

Fmoc-Phe(2-CF₃)-OH

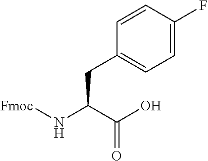

Fmoc-Phe(4-F)-OH

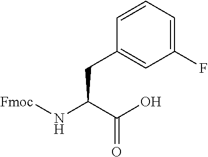

Fmoc-Phe(3-F)-OH

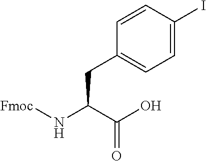

Fmoc-Phe(4-I)-OH

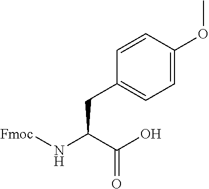

Fmoc-Tyr(Me)-OH
(Fmoc-Phe(4-OMe)-OH)

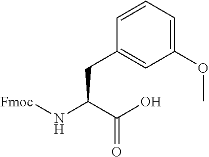

Fmoc-Phe(-3-OMe)-OH

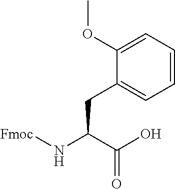

Fmoc-Phe(2-OMe)-OH

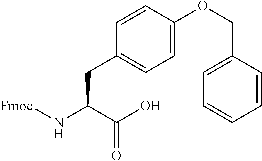

Fmoc-Tyr(Bn)-OH

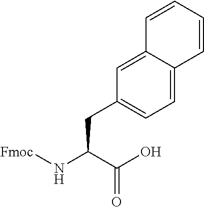

Fmoc-2-Nal-OH

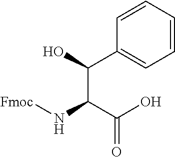

Fmoc-β-hydroxy-Phe-OH

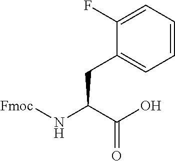

Fmoc-Phe(2-F)-OH

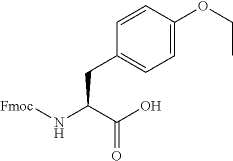

Fmoc-Tyr(Et)-OH

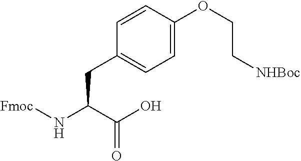

Fmoc-Tyr(CH₂CH₂NHBoc)-OH

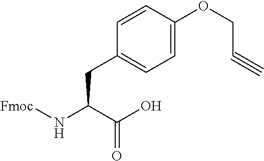

Fmoc-Tyr(propargyl)-OH

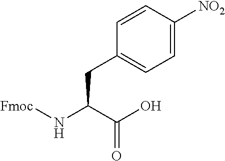

Fmoc-Phe(4-NO₂)-OH

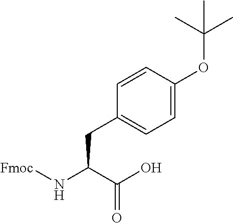

Fmoc-Tyr(tBu)-OH

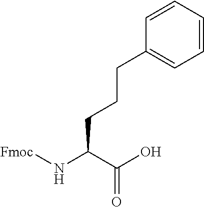

Fmoc-homoPhe-OH

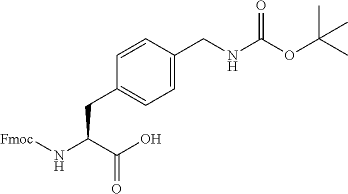

Fmoc-Phe(4-aminomethyl(Boc))-OH

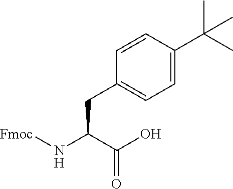

Fmoc-Phe(4-tBu)-OH

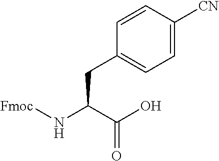

Fmoc-Phe(4-CN)-OH

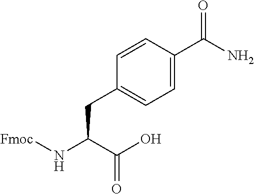

Fmoc-Phe(4-CONH₂)-OH

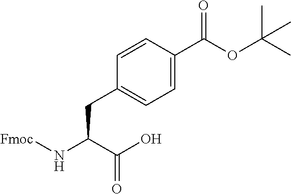

Fmoc-Phe(4-COOtBu)--OH

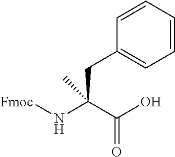

Fmoc-α-Me-Phe-OH

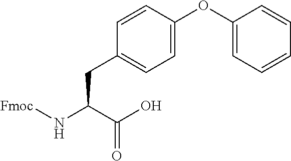

Fmoc-Phe(OPh)-OH
(Fmoc-Tyr(Ph)-OH)

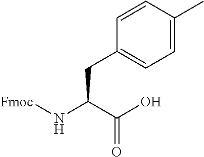

Fmoc-Phe(4-Me)-OH

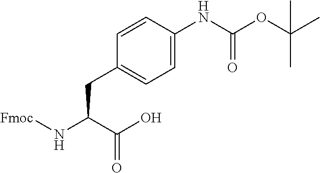

Fmoc-Phe(4-NHBoc)-OH

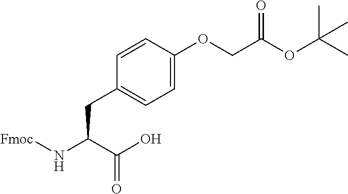

Fmoc-Tyr(CH₂COOtBu)-OH

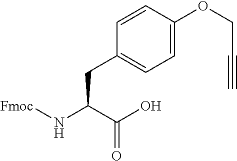

Fmoc-Tyr(propargyl)-OH

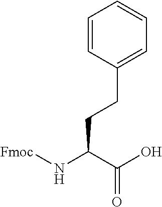

Fmoc-HomoPhe-OH

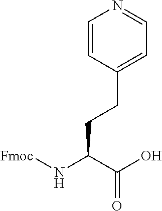

Fmoc-Homo-4-Py-Ala-OH

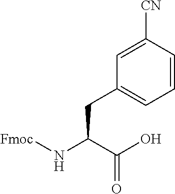

Fmoc-Phe(3-CN)-OH

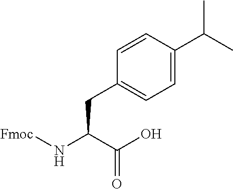

Fmoc-4-Phe(iPr)-OH

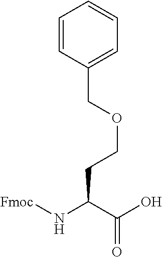

Fmoc-Homo-Ser(Bn)-OH

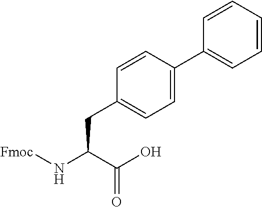

Fmoc-Bip-OH

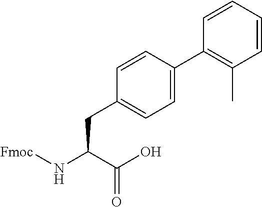

Fmoc-Bip(2′-Me)-OH

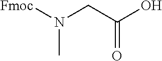

Fmoc-N-Me-Gly-OH
(Fmoc-mGly-OH)

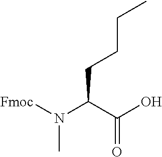

Fmoc-N-Me-Nle-OH
(Fmoc-mNle-OH)

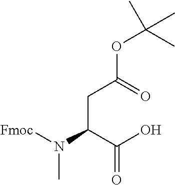

Fmoc-N-Me-Asp(OtBu)-OH
(Fmoc-mAsp(OtBu)-OH)

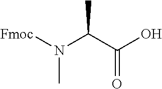

Fmoc-N-Me-Ala-OH
(Fmoc-mAla-OH)

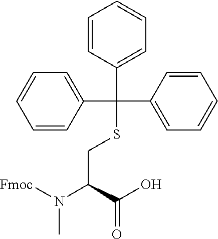

Fmoc-N-Me-Cys(Trt)-OH
(Fmoc-mCys(Trt)-OH)

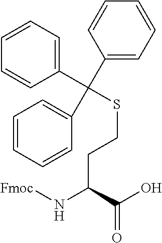

Fmoc-HomoCys(Trt)-OH

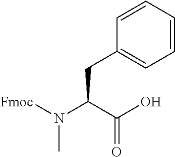

Fmoc-N-Me-Phe-OH
(Fmoc-mPhe-OH)

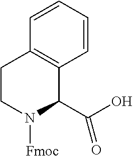

Fmoc-Iso-Tic-OH

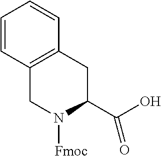

Fmoc-Tic-OH

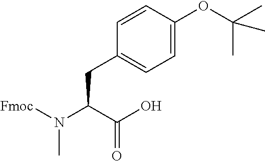

Fmoc-N-Me-Tyr(OtBu)-OH
(Fmoc0mTyr(OtBu)-OH)

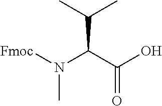

Fmoc-N-Me-Val-OH
(Fmoc-mVal-OH)

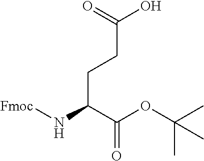

Fmoc-Glu-OtBu

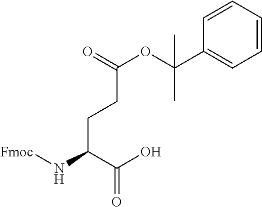

Fmoc-Glu(2-phenylisopropyloxy)-OH

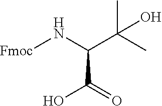

Fmoc-S-hydroxyvaline
Fmoc-V(β-OH)-OH

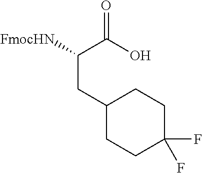

Fmoc-Ala(β-Cha(4,4-di-F))-OH

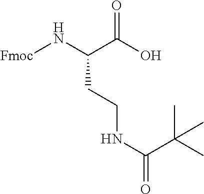

Fmoc-Dab(COtBu)-OH

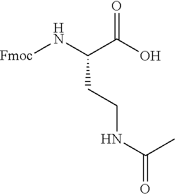

Fmoc-Dab(Ac)-OH

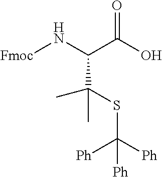

Fmoc-β,β-dimethyl-Cys(Trt)-OH
Fmoc-Pen(Trt)-OH

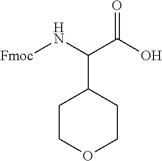

2-NHFmoc-2-(oxan-4-yl)acetic acid

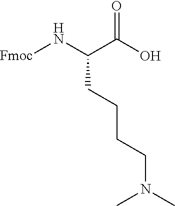

Fmo-Lys(N,N-di-Me)-OH

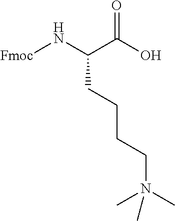

FMOC-Lys(triimethyl)-OH

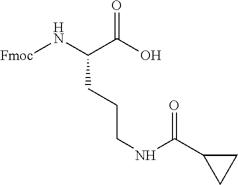

Fmoc-Orn(CO-cyclopropyl)-OH

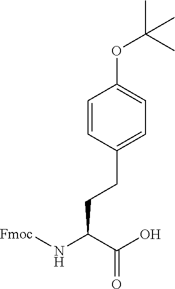

FMOC-HomoTyr(OtBu)-OH

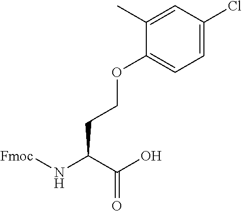

Fmoc-4-Cl-2-Me-phenoxyhomoSer-OH

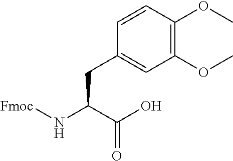

Fmoc-Phe(3,4-di-OMe)-OH

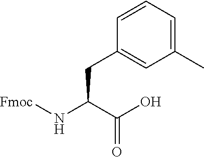

Fmoc-Phe(3-Me)-OH

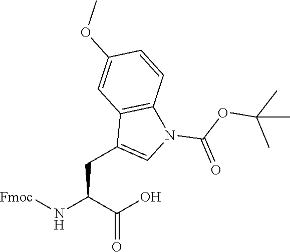

Fmoc-5-MeO-Trp(Boc)-OH

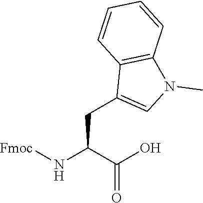

Fmoc-Nme-Trp-OH

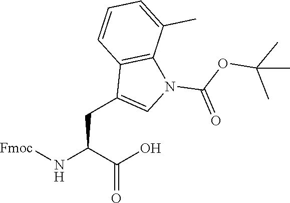

Fmoc-7-Me-Trp(Boc)-OH

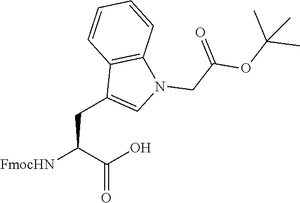

Fmoc-N-AcOH-Trp-OH

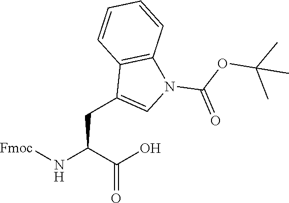

Fmoc-Trp(Boc)-OH

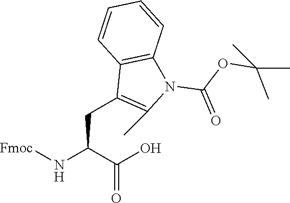

Fmoc-2-Me-Trp(Boc)-OH

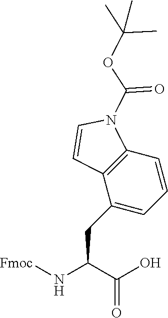

Fmoc-Iso-Trp(Boc)-OH

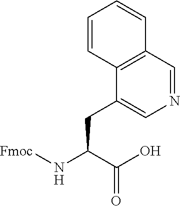

Fmoc-Ala-(β-isoquinolin-4-yl)-OH

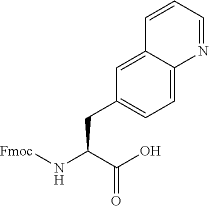

Fmoc-Ala-(β-quinolin-6-yl)-OH

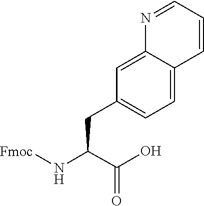

Fmoc-Ala-(-quinolin-7-yl)-OH

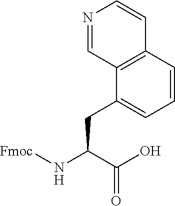

Fmoc-Ala-(β-isoquinolin-8-yl)-OH

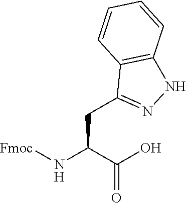

Fmoc-Ala(β-indazol-3-yl)-OH

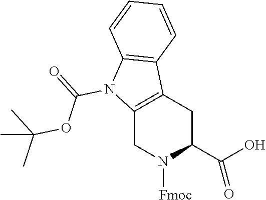

Fmoc-Trp(Boc)Tic-OH

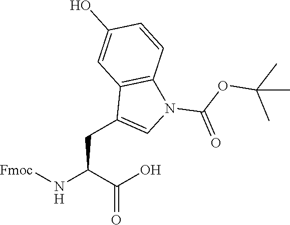

Fmoc-5-OH-Trp(Boc)-OH

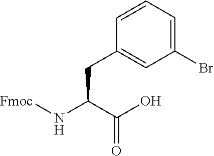

Fmoc-Phe(3-Br)-OH

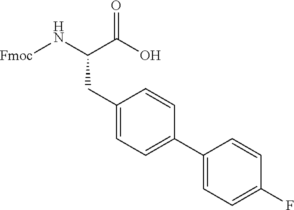

Fmoc-Bip(4′-Cl)-OH

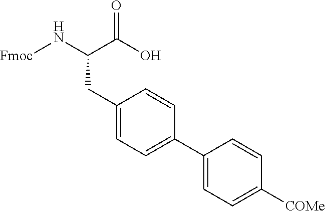

Fmoc-Bip(4′-COMe)-OH

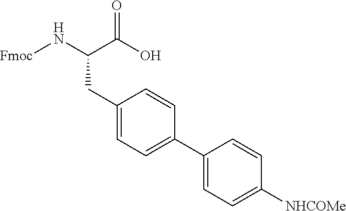

Fmoc-Bip(4′-NHCOMe)-OH

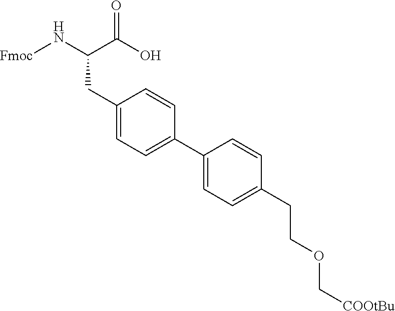

Fmoc-Bip(4′-
CH₂CH₂OCH₂COOtBu)-OH

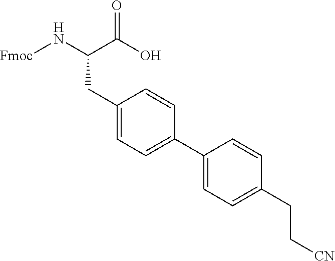

Fmoc-Bip(4′-CH₂CH₂CN)-OH

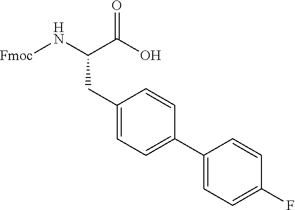

Fmoc-Bip(4′-F)-OH

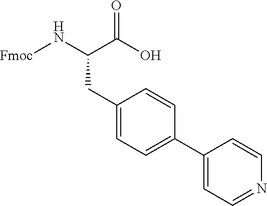

Fmoc-Bip(4′-Pyr)-OH

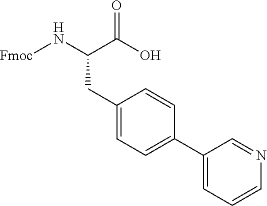

Fmoc-Bip(3′-Pyr)-OH

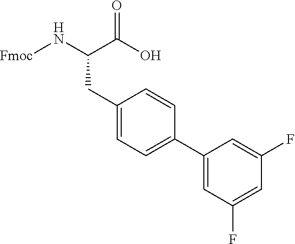

Fmoc-Bip(3′,5′-F2)-OH

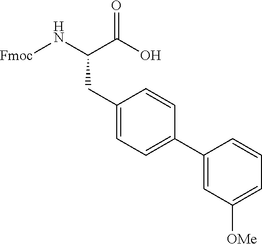

Fmoc-Bip(3′-OMe)-OH

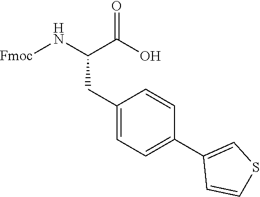

Fmoc-Bip(3′-Thio)-OH

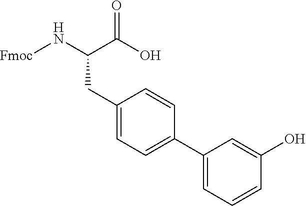

Fmoc-Bip(3′-OH)-OH

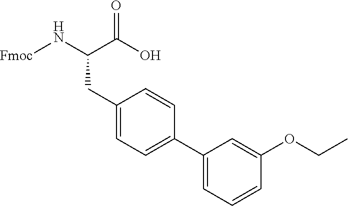

Fmoc-Bip(3′-OEt)-OH

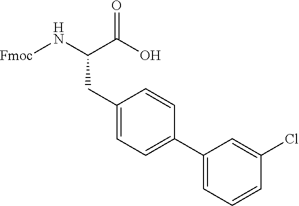

Fmoc-Bip(3′-CONH₂Cl)-OH

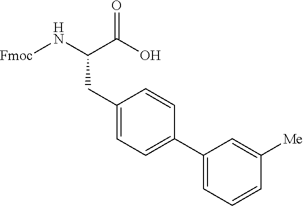

Fmoc-Bip(3′-Me)-OH

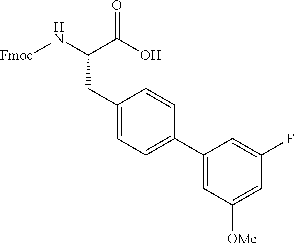

Fmoc-Bip(3′-F,5′-OMe)-OH

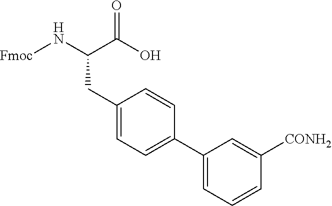

Fmoc-Bip(3′-CONH₂)-OH

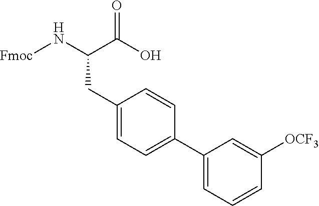

Fmoc-Bip(3′-OCF₃)-OH

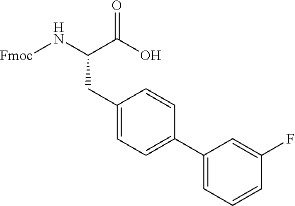

Fmoc-Bip(3′-F)-OH

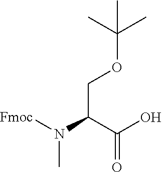

Fmoc-N-Me-Ser(tBu)-OH
(Fmoc-mSer(tBu)-OH)

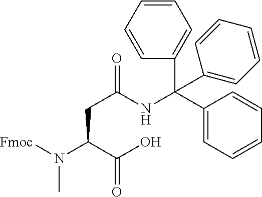

Fmoc-N-Me-Asn(Trt)-OH
(Fmoc-mAsn(Trt)-OH)

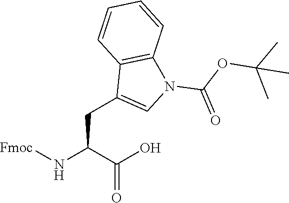

Fmoc-N-MeTrp(Boc)-OH
(Fmoc-mTrp(Boc)-OH)

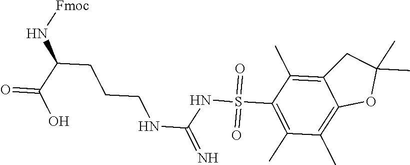

Fmoc-N-Me-Arg(Pbf)-OH
(Fmoc-mArf(Pbf)-OH)

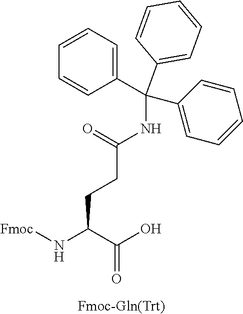

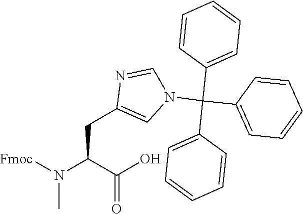

Fmoc-N-Me-His(Trt)

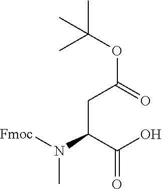

Fmoc-N-Me-Asp(OtBu)-OH
(Fmoc-mAsp(OtBu)-OH)

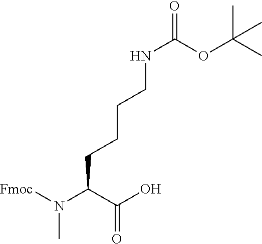

Fmoc-N-Me-Lys(Boc)-OH
(Fmoc-mLys(Boc)-OH)

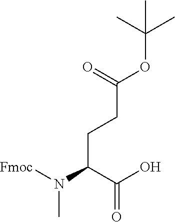

Fmoc-N-Me-Glu(OtBu)-OH
(Fmoc-nGlu(OtBu)-OH)

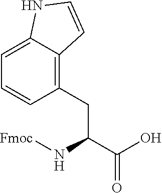

2-(Fmoc-amino)-3-(1H-indol-4-
yl)propanoic acid

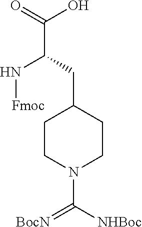

Fmoc-Ala(β-1-
carbamimidoylpiperidin-4-
yl)-OH

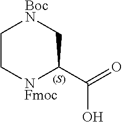

(S)-1-Fmoc-4-Boc-
piperazine-2-carboxylic acid

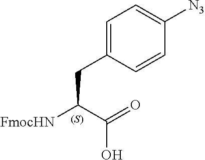

Fmoc-Phe(4-N₃)-OH

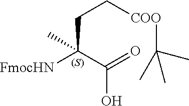

Fmoc-α-Me-Glu(tBu)

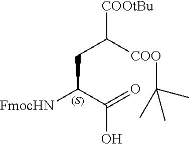

Fmoc-β-COOtBu-Glu(tBu)

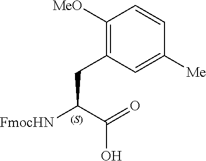

Fmoc-Phe(2-OMe-5-Me)-OH

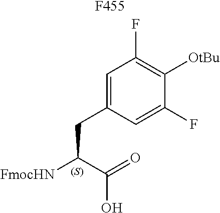

Fmoc-Tyr(tBu, 2,6-di-F)-OH

### Analytical Data:

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “m z” unit designation. Compounds with exact masses greater than 1000 were often detected as double-charged or triple-charged ions.

The crude material was purified via preparative LC/MS. Fractions containing the desired product were combined and dried via centrifugal evaporation.

### Analytical LC/MS Condition A:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LC/MS Condition B:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

### Analytical LC/MS Condition C:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 70° C.; Gradient: 0-100% B over 3 minutes, then a 2.0-minute hold at 100% B; Flow: 0.75 mL/min; Detection: UV at 220 nm.

### Analytical LC/MS Condition D:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 70° C.; Gradient: 0-100% B over 3 minutes, then a 2.0-minute hold at 100% B; Flow: 0.75 mL/min; Detection: UV at 220 nm.

### Analytical LC/MS Condition E:

Column: Kinetex XB C18, 3.0×75 mm, 2.6-μm particles; Mobile Phase A: 10 mM ammonium formate in water: acetonitrile (98:2); Mobile Phase B: 10 mM ammonium formate in Water: acetonitrile (02:98); Gradient: 20-100% B over 4 minutes, then a 0.6-minute hold at 100% B; Flow: 1.0 mL/min; Detection: UV at 254 nm.

### Analytical LC/MS Condition F:

Column: Ascentis Express C18, 2.1×50 mm, 2.7-μm particles; Mobile Phase A: 10 mM ammonium acetate in water: acetonitrile (95:5); Mobile Phase B: 10 mM ammonium acetate in Water: acetonitrile (05:95), Temperature: 50° C.; Gradient: 0-100% B over 3 minutes; Flow: 1.0 mL/min; Detection: UV at 220 nm.

### Analytical LC/MS Condition G:

Column: X Bridge C18, 4.6×50 mm, 5-μm particles; Mobile Phase A: 0.1% TFA in water; Mobile Phase B: acetonitrile, Temperature: 35° C.; Gradient: 5-95% B over 4 minutes; Flow: 4.0 mL/min; Detection: UV at 220 nm.

### Analytical LC/MS Condition H:

Column: X Bridge C18, 4.6×50 mm, 5-μm particles; Mobile Phase A: 10 mM NH₄OAc; Mobile Phase B: methanol, Temperature: 35° C.; Gradient: 5-95% B over 4 minutes; Flow: 4.0 mL/min; Detection: UV at 220 nm.

### Analytical LC/MS Condition I:

Column: X Bridge C18, 4.6×50 mm, 5-μm particles; Mobile Phase A: 10 mM NH₄OAc; Mobile Phase B: acetonitrile, Temperature: 35° C.; Gradient: 5-95% B over 4 minutes; Flow: 4.0 mL/min; Detection: UV at 220 nm.

### Analytical LC/MS Condition J:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 70° C.; Gradient: 0-100% B over 1.5 minutes, then a 2.0-minute hold at 100% B; Flow: 0.75 mL/min; Detection: UV at 254 nm.

### Analytical LC/MS Condition K:

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 1.0 minutes, then at 1.0-1.5 minute hold at 100% B; Flow: 0.80 mL/min; Detection: UV at 220 nm.

### Analytical LC/MS Condition L:

Column: Waters Acquity UPLC BEH C18, 2.1×50 mm, 1.7-μm particles; Buffer: 10 mM Ammonium Acetate. Mobile Phase A: buffer” CH₃CN (95/5); Mobile Phase B: Mobile Phase B: Buffer: ACN (5:95); Temperature: 50° C.; Gradient: 20-98% B over 2.0 minutes, then at 0.2 minute hold at 100% B; Flow: 0.70 mL/min; Detection: UV at 220 nm.

### Analytical LC/MS Condition M:

Column: Waters Acquity UPLC BEH C18, 3.0×50 mm, 1.7-μm particles; Mobile Phase A: 95% water and 5% water with 0.1% trifluoroacetic acid; Mobile Phase B: 95% acetonitrile and 5% water with 0.1% trifluoroacetic acid; Temperature: 50° C.; Gradient: 20-100% B over 2.0 minutes, then at 2.0-2.3 minute hold at 100% B; Flow: 0.7 mL/min; Detection: UV at 220 nm.

### General Procedures:

### Prelude Method:

All manipulations were performed under automation on a Prelude peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl) (4-alkoxyphenyl) methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl) polystyrene.

Common amino acids used are listed below with side-chain protecting groups indicated inside parenthesis: Fmoc-Ala-OH; Fmoc-Arg(Pbf)-OH; Fmoc-Asn Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Prelude Method” describe an experiment performed on a 0.100 mmol scale, where the scale is determined by the amount of Sieber or Rink or 2-chlorotrityl or PL-FMP resin. This scale corresponds to approximately 140 mg of the Sieber amide resin described above. All procedures can be scaled down from the 0.100 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine: DMF (20:80 v/v, 5.0 mL). The mixture was periodically agitated for 5.0 minutes and then the solution was drained through the frit. To the reaction vessel was added piperidine: DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Double-Coupling Procedure:

To the reaction vessel containing the resin from the previous step was added piperidine: DMF (20:80 v/v, 5.0 mL). The mixture was periodically agitated for 5.0 minutes and then the solution was drained through the frit. To the reaction vessel was added piperidine: DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hour, then the reaction solution was drained through the frit. The resin was washed successively two times as follows: for each wash, DMF (5.0 mL) was added through the top of the vessel and the resulting mixture was periodically agitated for 1.0 minute before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hours, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Single-Coupling Manual Addition Procedure A:

To the reaction vessel containing the resin from the previous step was added piperidine: DMF (20:80 v/v, 5.0 mL). The mixture was periodically agitated for 5 minutes and then the solution was drained through the frit. To the reaction vessel was added piperidine: DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-2 mL) was added manually using a pipette from the top of the vessel while the bottom of the vessel was remain attached to the instrument, then the vessel was closed. The automatic program was resumed and HATU (0.4 M in DMF, 1.3 mL, 4 equiv) and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine: DMF (20:80 v/v, 5.0 mL). The mixture was periodically agitated for 5 minutes and then the solution was drained through the frit. To the reaction vessel was added piperidine: DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, followed by the manual addition of HATU (2-4 equiv, same equiv as the unnatural amino acid), and then the vessel was closed. The automatic program was resumed and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine: DMF (20:80 v/v, 5.0 mL). The mixture was periodically agitated for 5 minutes and then the solution was drained through the frit. To the reaction vessel was added piperidine: DMF (20:80 v/v, 5.0 mL). The mixture was periodically agitated for 5 minutes and then the solution was drained through the frit. The resin was washed successively six times as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed twice as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed successively five times as follows: for each wash, DMF (6.0 mL) was added through the top of the vessel and the resulting mixture was periodically agitated for one minute before the solution was drained through the frit. The resin was washed successively four times as follows: for each wash, DCM (6.0 mL) was added through the top of the vessel and the resulting mixture was periodically agitated for one minute before the solution was drained through the frit. The resin was then dried with nitrogen flow for 10 minutes. The resulting resin was used directly in the next step.

### Symphony Method:

All manipulations were performed under automation on a 12-channel Symphony peptide synthesizer (Protein Technologies). Unless noted, all procedures were performed in a 25-mL polypropylene reaction vessel fitted with a bottom frit. The reaction vessel connects to the Symphony peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution were used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl) (4-alkoxyphenyl) methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading.

PL-FMP resin: (4-Formyl-3-methoxyphenoxymethyl) polystyrene.

Fmoc-glycine-2-chlorotrityl chloride resin, 200-400 mesh, 1% DVB, 0.63 mmol/g loading.

Common amino acids used are listed below with side-chain protecting groups indicated inside parenthesis: Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony Method” describe an experiment performed on a 0.05 mmol scale, where the scale is determined by the amount of Sieber or Rink or chlorotrityl linker or PL-FMP bound to the resin. This scale corresponds to approximately 70 mg of the Sieber resin described above. All procedures can be scaled up from the 0.05 mmol scale by adjusting the described volumes by the multiple of the scale.

Prior to the amino acid coupling, all peptide synthesis sequences began with a resin-swelling procedure, described below as “Resin-swelling procedure”. Coupling of amino acids to a primary amine N-terminus used the “Single-coupling procedure” described below.

### Resin-Swelling Procedure:

To a 25-mL polypropylene solid-phase reaction vessel was added the resin (0.05 mmol). The resin was washed (swelled) as follows: to the reaction vessel was added DMF (2.0-3.0 mL, 1-2 times), upon which the mixture was periodically agitated for 10 minutes before the solvent was drained through the frit. Sometimes the resin was washed (swelled) as follows: to the reaction vessel was added CH₂Cl₂ (3-5 mL, 2 times) and upone which the mixture was periodically agitated for 30 min and before the solvent was drained through the frit. Then DMF (2.0-3.0 mL, 1-6 times), upon which the mixture was periodically agitated for 2-10 minutes before the solvent was drained through the frit.

### Single-Coupling Procedure:

To the reaction vessel containing the resin from the previous step was added DMF (2.5-3.75 mL) three times, upon which the mixture was agitated for 30 seconds before the solvent was drained through the frit each time. To the resin was added piperidine: DMF (20:80 v/v, 3.0-3.75 mL). The mixture was periodically agitated for 5.0 minutes and then the solution was drained through the frit. To the reaction vessel was added piperidine: DMF (20:80 v/v, 3.0-3.75 mL). The mixture was periodically agitated for 5.0 minutes and then the solution was drained through the frit. Sometimes the deprotection step was performed the third time. The resin was washed successively six times as follows: for each wash, DMF (2.5-3.75 mL) was added to the vessel and the resulting mixture was periodically agitated for 30 seconds before the solution was drained through the frit. To the reaction vessel was added the amino acid (0.2 M in DMF, 2.0-2.5 mL, 8-10 equiv), then HATU (0.4 M in DMF, 1.0-1.25 mL, 8-10 equiv), and finally NMM (0.8 M in DMF, 1.0-1.25 mL, 20 equiv). The mixture was periodically agitated for 30-120 minutes, then the reaction solution was drained through the frit. The resin was washed successively six times as follows: for each wash, DMF (2.5-3.0 mL) was added and the resulting mixture was periodically agitated for 30 seconds before the solution was drained through the frit. The resulting resin was used directly in the next step.

### Single-Coupling Manual Addition Procedure:

To the reaction vessel containing the resin from the previous step was added DMF (3.0-3.75 mL) three times, upon which the mixture was agitated for 30 seconds before the solvent was drained through the frit each time. To the resin was added piperidine: DMF (20:80 v/v, 3.0-3.75 mL). The mixture was periodically agitated for 5.0 minutes and then the solution was drained through the frit. To the reaction vessel was added piperidine: DMF (20:80 v/v, 3.0-3.75 mL). The mixture was periodically agitated for 5.0 minutes and then the solution was drained through the frit. The mixture was periodically agitated for 5.0 minutes and then the solution was drained through the frit. The resin was washed successively six times as follows: for each wash, DMF (3.0-3.75 mL) was added to the vessel and the resulting mixture was periodically agitated for 30 seconds before the solution was drained through the frit. To the reaction vessel was added the premixed amino acid (2.0-5.0 equiv) and HATU (0.4 M in DMF, 2.0-5.0 equiv), then NMM (0.8 M in DMF, 4.0-10.0 equiv) and the molar ratio for amino acid, HATU, and NMM is 1:1:2. The mixture was periodically agitated for 2-6 hours, then the reaction solution was drained through the frit. The resin was washed successively four times as follows: for each wash, DMF (3.75 mL) was added and the resulting mixture was periodically agitated for 30 seconds before the solution was drained through the frit. The resulting resin was used directly in the next step.

### Double-Coupling Procedure:

To the reaction vessel containing resin from the previous step was added DMF (2.5-3.75 mL) three times, upon which the mixture was agitated for 30 seconds before the solvent was drained through the frit each time. To the reaction vessel was added piperidine: DMF (20:80 v/v, 3.0-3.75 mL). The mixture was periodically agitated for 5 minutes and then the solution was drained through the frit. To the reaction vessel was added piperidine: DMF (20:80 v/v, 3.0-3.75 mL). The mixture was periodically agitated for 5 minutes and then the solution was drained through the frit. The resin was washed successively six times as follows: for each wash, DMF (3.0-3.75 mL) was added and the resulting mixture was periodically agitated for 30 seconds before the solution was drained through the frit. To the reaction vessel was added the amino acid (0.2 M in DMF, 2.0-2.5 mL, 8-10 equiv), then HATU (0.4 M in DMF, 1.0-1.25 mL, 10 equiv), and finally NMM (0.8 M in DMF, 1.0-1.25 mL, 16-20 equiv). The mixture was periodically agitated for 1 hour, then the reaction solution was drained through the frit. The resin was washed twice with DMF (3.0-3.75 mL) and the resulting mixture was periodically agitated for 30 seconds before the solution was drained through the frit each time. To the reaction vessel was added the amino acid (0.2 M in DMF, 2.0-2.5 mL, 8-10 equiv), then HATU (0.4 M in DMF, 1.0-1.25 mL, 8-10 equiv), and finally NMM (0.8 M in DMF, 1.0-1.25 mL, 16-20 eq). The mixture was periodically agitated for 1-2 hours, then the reaction solution was drained through the frit. The resin was successively washed six times as follows: for each wash, DMF (3.0-3.75 mL) was added and the resulting mixture was periodically agitated for 30 seconds before the solution was drained through the frit. The resulting resin was used directly in the next step.

### Chloroacetic Anhydride Coupling:

To the reaction vessel containing resin from the previous step was added DMF (3.0-3.75 mL) three times, upon which the mixture was agitated for 30 seconds before the solvent was drained through the frit each time. To the reaction vessel containing the resin from the previous step was added piperidine: DMF (20:80 v/v, 3.0-3.75 mL). The mixture was periodically agitated for 5 minutes and then the solution was drained through the frit. To the reaction vessel was added piperidine: DMF (20:80 v/v, 3.0-3.75 mL). The mixture was periodically agitated for 5 minutes and then the solution was drained through the frit. The resin was washed successively six times as follows: for each wash, DMF (3.0-3.75 mL) was added through the top of the vessel and the resulting mixture was periodically agitated for 30 seconds before the solution was drained through the frit. To the reaction vessel was added the chloroacetic anhydride solution (0.4 M in DMF, 3.0-3.75 mL, 30 equiv), then NMM (0.8 M in DMF, 2.5 mL, 40 equiv). The mixture was periodically agitated for 15 minutes, then the reaction solution was drained through the frit. The resin was washed once as follows: DMF (5.0-6.25 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NMM (0.8 M in DMF, 2.5 mL, 40 equiv). The mixture was periodically agitated for 15 minutes, then the reaction solution was drained through the frit. The resin was washed successively six times as follows: for each wash, DMF (2.5 mL) was added through the top of the vessel and the resulting mixture was periodically agitated for 30 seconds before the solution was drained through the frit. The resin was washed successively four times as follows: for each wash, DCM (2.5 mL) was added through the top of the vessel and the resulting mixture was periodically agitated for 30 seconds before the solution was drained through the frit. The resulting resin was dried using a nitrogen flow for 10 mins before being used directly in the next step.

### Symphony X Methods:

All manipulations were performed under automation on a Symphony X peptide synthesizer (Protein Technologies). Unless noted, all procedures were performed in a 45-mL polypropylene reaction vessel fitted with a bottom frit. The reaction vessel connects to the Symphony X peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. A “single shot” mode of addition describes the addition of all the solution contained in the single shot falcon tube that is usually any volume less than 5 mL. Amino acid solutions were generally not used beyond two weeks from preparation. HATU solution was used within 14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl) (4-alkoxyphenyl) methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl) polystyrene.

Common amino acids used are listed below with side-chain protecting groups indicated inside parenthesis: Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony X Method” describe an experiment performed on a 0.050 mmol scale, where the scale is determined by the amount of Sieber or Rink or 2-chlorotrityl or PL-FMP bound to the resin. This scale corresponds to approximately 70 mg of the Sieber amide resin described above. All procedures can be scaled beyond or under 0.050 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- or D-Leu used the “Double-coupling procedure” or the “Single-Coupling 2-Hour Procedure” described below. Unless otherwise specified, the last step of automated synthesis is the acetyl group installation described as “Chloroacetyl Anhydride Installation”. All syntheses end with a final rinse and drying step described as “Standard final rinse and dry procedure”.

### Resin-Swelling Procedure:

To a 45-mL polypropylene solid-phase reaction vessel was added Sieber amide resin (70 mg, 0.050 mmol). The resin was washed (swelled) three times as follows: to the reaction vessel was added DMF (5.0 mL) through the top of the vessel “DMF top wash” upon which the mixture was periodically agitated for 3 minutes before the solvent was drained through the frit.

### Single-Coupling Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure A:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, then the vessel was closed. The automatic program was resumed and HATU (0.4 M in DMF, 1.0 mL, 8 equiv) and NMM (0.8 M in DMF, 1.0 mL, 16 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, followed by the manual addition of HATU (2-4 equiv, same equiv as the unnatural amino acid), then the vessel was closed. The automatic program was resumed and NMM (0.8 M in DMF, 1.0 mL, 16 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine: DMF (20:80 v/v, 3.0 mL). The mixture was periodically agitated for 3.5 or 5 minutes and then the solution was drained through the frit. To the reaction vessel was added piperidine: DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for 1.0 minute before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for 1.0 minute before the solution was drained through the frit. The resulting resin was used directly in the next step.

### Final Rinse and Dry Procedure:

The resin from the previous step was washed successively six times as follows: for each wash, DCM (5.0 mL) was added through the top of the vessel and the resulting mixture was periodically agitated for 30 seconds before the solution was drained through the frit. The resin was then dried using a nitrogen flow for 10 minutes. The resulting resin was used directly in the next step.

### Global Deprotection Method A:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL falcon tube was added the resin and 2.0-5.0 mL of the cleavage cocktail (TFA: TIS: DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et₂O (30-40 mL); then the mixture was centrifuged for 3-5 minutes; and the solution was decanted away from the solids and discarded. For a final time, the solids were suspended in Et₂O (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 30-ml bio-rad poly-prep chromatography column was added the resin and 2.0-5.0 mL of the cleavage cocktail (TFA: TIS: DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA. The mixture was centrifuged for 3-5 minutes, then the solution was decanted away from the solids and discarded. The solids were suspended in Et₂O (35 mL); then the mixture was centrifuged for 3-5 minutes; and the solution was decanted away from the solids and discarded. For a final time, the solids were suspended in Et₂O (35 mL); the mixture was centrifuged for 3-5 minutes; and the solution was decanted away from the solids and discarded to afford the crude peptide as a white to off-white solid after drying under a flow of nitrogen and/or under house vacuum. The crude was used at the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the globle deprotection were dissolved in DMF (30-45 mL) in the 50-mL centrifuge tube at room temperature, and to the solution was added DIEA (1.0-2.0 mL) and the pH value of the reaction mixture above was 8. The solution was then allowed to shake for several hours or overnight or over 2-3 days at room temperature. The reaction solution was concentrated to dryness on speedvac or genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in CH₃CN/0.1 M aqueous solution of ammonium bicarbonate (1:1, v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on speedvac or genevac EZ-2. The resulting residue was charged with CH₃CN:H₂O (2:3, v/v, 30 mL), and concentrated to dryness on speedvac or genevac EZ-2. This procedure was repeated (usually 2 times). The resulting crude solids were then dissolved in DMF or DMF/DMSO or CH₃CN/H₂O/formic acid. After filtration, the solution was subjected to single compound reverse-phase HPLC purification to afford the desired cyclic peptide.

### N-Methylation On-Resin Method A.

To the resin (50 μmol) in a Bio-Rad tube was added CH₂Cl₂ (2 mL) and shaken for 5 min at rt. 2-Nitrobenzene-1-sulfonyl chloride (44.3 mg, 200 μmol, 4 equiv) was added followed by the addition of 2,4,6-trimethylpyridine (0.040 mL, 300 μmol, 6 equiv). The reaction was shaken at rt for 2 h. The solvent was drained and the resin was rinsed with CH₂C₁₂ (5 mL×3), DMF (5 mL×3) and then THF (5 mL×3). The resin was added THF (1 mL). Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 2-16 h. The reaction was repeated. Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 1-16 h. The solvent was drained, and the resin was washed with THF (5 mL×3) and CHCl₃ (5 mL×3). The resin was air dried and used directly in the next step. The resin was shaken in DMF (2 mL). 2-Mercaptoethanol (39.1 mg, 500 μmol) was added followed by DBU (0.038 mL, 250 μmol, 5 equiv). The reaction was shaken for 1.5 h. The solvent was drained. The resin was washed with DMF (4×). Air dried and used directly in the next step.

N-Methylation on-resin Method B (Turner, R. A. et al, Org. Lett., 15 (19): 5012-5015 (2013)). All manipulations were performed manually unless noted. The procedure of “N-methylation on-resin Method A” describes an experiment performed on a 0.100 mmol scale, where the scale is determined by the amount of Sieber or Rink linker bound to the resin that was used to generate the peptide. This scale is not based on a direct determination of the quantity of peptide used in the procedure. The procedure can be scaled beyond 0.10 mmol scale by adjusting the described volumes by the multiple of the scale. The resin was transferred into a 25 mL fritted syringe. To the resin was added piperidine: DMF (20:80 v/v, 5.0 mL). The mixture was shaken for 3 min. and then the solution was drained through the frit. The resin was washed 3 times with DMF (4.0 mL). To the reaction vessel was added piperidine: DMF (20:80 v/v, 4.0 mL). The mixture was shaken for 3 min. and then the solution was drained through the frit. The resin was washed successively three times with DMF (4.0 mL) and three times with DCM (4.0 mL). The resin was suspended in DMF (2.0 mL) and ethyl trifluoroacetate (0.119 ml, 1.00 mmol), 1,8-diazabicyclo[5.4.0]undec-7-ene (0.181 ml, 1.20 mmol). The mixture was placed on a shaker for 60 min. The solution was drained through the frit. The resin was washed successively three times with DMF (4.0 mL) and three times with DCM (4.0 mL). The resin was washed three times with dry THF (2.0 mL) to remove any residual water. In an oven dried 4.0 mL vial was added THF (1.0 mL) and triphenylphosphine (131 mg, 0.500 mmol) on dry 4 Å molecular sieves (20 mg). The solution was transferred to the resin and diisopropyl azodicarboxylate (0.097 mL, 0.5 mmol) was added slowly. The resin was stirred for 15 min. The solution was drained through the frit and the resin was washed with three times with dry THF (2.0 mL) to remove any residual water. In an oven dried 4.0 mL vial was added THF (1.0 mL), triphenylphosphine (131 mg, 0.50 mmol) on dry 4 Å molecular sieves (20 mg). The solution was transferred to the resin and diisopropyl azodicarboxylate (0.097 mL, 0.5 mmol) was added slowly. The resin was stirred for 15 min. The solution was drained through the frit. The resin was washed successively three times with DMF (4.0 mL) and three times with DCM (4.0 mL). The resin was suspended in Ethanol (1.0 mL) and THF (1.0 mL), and sodium borohydride (37.8 mg, 1.000 mmol) was added. The mixture was stirred for 30 min. and drained. The resin was washed successively three times with DMF (4.0 mL) and three times with DCM (4.0 mL).

### N-Alkylation On-Resin Procedure Method A:

A solution of the alcohol corresponding to the alkylating group (0.046 g, 1.000 mmol), triphenylphosphine (0.131 g, 0.500 mmol), and DIAD (0.097 mL, 0.500 mmol) in 3 mL of THF was added to nosylated resin (0.186 g, 0.100 mmol), and the reaction mixture was stirred for 16 hours at room temperature. The resin was washed three times with THF (5 mL) Tetrahydrofuran, and the above procedure was repeated 1-3 times. Reaction progress was monitored by TFA micro-cleavage of small resin samples treated with a solution of 50 μL of TIS in 1 mL of TFA for 1.5 hours.

### N-Alkylation On-Resin Procedure Method B:

The nosylated resin (0.100 mmol) was washed three times with N-methylpyrrolidone (NMP) (3 mL). A solution of NMP (3 mL), Alkyl Bromide (20 eq, 2.000 mmol) and DBU (20 eq, 0.301 mL, 2.000 mmol) was added to the resin, and the reaction mixture was stirred for 16 hours at room temperature. The resin was washed with NMP (3 mL) and the above procedure was repeated once more. Reaction progress was monitored by TFA micro-cleavage of small resin samples treated with a solution of 50 μL of TIS in 1 mL of TFA for 1.5 hours.

### N-Nosylate Formation Procedure:

A solution of collidine (10 eq.) in DCM (2 mL) was added to the resin, followed by a solution of Nos-C₁ (8 eq.) in DCM (1 mL). The reaction mixture was stirred for 16 hours at room temperature. The resin was washed three times with DCM (4 mL) and three times with DMF (4 mL). The alternating DCM and DMF washes were repeated three times, followed by one final set of four DCM washes (4 mL).

### N-Nosylate Removal Procedure:

The resin (0.100 mmol) was swelled using three washes with DMF (3 mL) and three washes with NMP (3 mL). A solution of NMP (3 mL), DBU (0.075 mL, 0.500 mmol) and 2-mercaptoethanol (0.071 mL, 1.000 mmol) was added to the resin and the reaction mixture was stirred for 5 minutes at room temperature. After filtering and washing with NMP (3 mL), the resin was re-treated with a solution of NMP (3 mL), DBU (0.075 mL, 0.500 mmol) and 2-mercaptoethanol (0.071 mL, 1.000 mmol) for 5 minutes at room temperature. The resin was washed three times with NMP (3 mL), four times with DMF (4 mL) and four times with DCM (4 mL), and was placed back into a Symphony reaction vessel for completion of sequence assembly on the Symphony peptide synthesizer.

### General Procedure for Preloading Amines on the PL-FMP Resin:

PL-FMP resin (Novabiochem, 1.00 mmol/g substitution) was swollen with DMF (20 mL/mmol) at room temperature. The solvent was drained and 10 ml of DMF was added, followed by the addition of the amine (2.5 mmol) and acetic acid (0.3 mL) into the reaction vessel. After 10-min agitation, sodium triacetoxyhydroborate (2.5 mmol) was added. The reaction was allowed to agitate overnight. The resin was washed by DMF (1×), THF/H₂O/AcOH (6:3:1) (2×), DMF (2×), DCM (3×), and dried. The resulting PL-FMP resin preloaded with the amine can be checked by the following method: Took 100 mg of above resin and reacted with benzoyl chloride (5 equiv), and DIEA (10 equiv) in DCM (2 mL) at room temperature for 0.5 h. The resin was washed with DMF (2×), MeOH (1×), and DCM (3×). The sample was then cleaved with 40% TFA/DCM (1 h). The product was collected and analyzed by HPLC and MS. Collected sample was dried and got weight to calculate resin loading.

### General Procedure for Preloading (Fmocamino) Acisa on Cl-Trityl Resin:

To a glass reaction vessel equipped with a frit was added the 2-Chloro-chlorotrityl resin mesh 50-150, (1.54 meq/gram, 1.94 grams, 3.0 mmole) to be swollen in DCM (5 mL) for 5 minutes. A solution of the acid (3.00 mmol, 1.0 eq) in DCM (5 mL) was added to the resin followed by DIPEA (2.61 ml, 15.00 mmol, 5.0 eq). The reaction was shaken at room temperature for 60 minutes. Add in DIEA (0.5 mL) and methanol (3 mL), shaken for an additional 15 minutes. The reaction solution was filtered through the frit and the resin was rinsed with DCM (4×5 mL), DMF (4×5 mL), DCM (4×5 mL), diethyl ether (4×5 mL), and dried using a flow of nitrogen. The resin loading can be determined as follows:

A sample of resin (13.1 mg) was treated with 20% piperidine/DMF (v/v, 2.0 mL) for 10 minutes with shaking. 1 mL of this solution was transferred to a 25.0 mL volumetric flask and diluted with methanol to a total volume of 25.0 mL. A blank solution of 20% piperidine/DMF (v/v, 1.0 mL) was diluted up with methanol in a volumetric flask to 25.0 mL. The UV was set to 301 nm and zero with the blank solution followed by the reading of the solution,
Absorbance=1.9411
(1.9411/20 mg)*6.94=0.6736. Loading of the resin was measured to be 0.6736 mmol/g.

### Click Reaction On-Resin Procedure Method A:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2×5 mL×5 mins). In a 200-ml bottle was charged with 30 time of the following: vitamin C (0.026 g, 0.150 mmol), bis(2,2,6,6-tetramethyl-3,5-heptanedionato) copper (II) (10.75 mg, 0.025 mmol), DMF (1.5 mL), 2,6-lutidine (0.058 mL, 0.50 mmol) and THF (1.5 ml), followed by DIPEA (0.087 ml, 0.50 mmol) and the azide, tert-butyl(S)-1-azido-40-(tert-butoxycarbonyl)-37,42-dioxo-3,6,9, 12, 15, 18,21,24,27,30,33-undecaoxa-36,41-diazanonapentacontan-59-oate (0.028 g, 0.025 mmol). The mixture was stirred until everything was in solution. The DMF in the above Bio-Rad tube was drained, and the above click solution (3 mL each) was added to each Bio-Rad tube. The tubes were shaken overnight on an orbital shaker. Solutions were drained through the frit. The resins were washed with DMF (3×2 mL) and DCM (3×2 mL).

### Click Reaction On-Resin Procedure Method B:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2 5 mL×5 mins). In a separate bottle, nitrogen was bubbled into 4.0 mL of DMSO for 15 mins. To the DMSO was added copper iodide (9.52 mg, 0.050 mmol, 1.0 eq) (sonicated), lutidine (58 μL, 0.500 mmol, 10.0 eq) and DIEA (87 uL, 0.050 mmol, 10.0 eq). The solution was purged with nitrogen again. DCM was drained through the frit. In a separate vial, ascorbic acid (8.8 mg, 0.050 mmol, 1.0 eq) was dissolved into water (600 uL). Nitrogen was bubbled through the solution for 10 mins. Coupling partners were distributed in the tubes (0.050 mmol to 0.10 mmol, 1.0 to 2.0 eq) followed by the DMSO copper and base solution and finally ascorbic acid aqueous solution. The solutions were topped with a blanket of nitrogen and capped. The tube was put onto the rotatory mixer for 16 hours. Solutions were drained through the frit. The resins were washed with DMF (3×2 mL) and DCM (3×2 mL).

### Suzuki Reaction On-Resin Procedure:

In a Bio Rad tube is placed 50 μmoles of dried Rink resin of a N-terminus Fmoc-protected linear polypeptide containing a 4-bromo-phenylalanine side chain. The resin was swelled with DMF (2×5 mL). To this was added a DMF solution (2 mL) of p-tolylboronic acid (0.017 g, 0.125 mmol), potassium phosphate (0.2 mL, 0.400 mmol) followed by the catalyst [1,1′-bis(di-tert-butylphosphino) ferrocene]dichloropalladium (II) [PdCl₂(dtbpf)] (3.26 mg, 5.00 μmol). The tube was shaken at rt overnight. The solution was drained and the resin was washed with DMF (5×3 mL) followed by alternating DCM (2×3 mL), then DMF (2×3 mL), and then DCM (5×3 mL). A small sample of resin was micro-cleaved using 235 μL of TIS in 1 ml TFA at rt for 1 h. The rest of the resin was used in the next step of peptide coupling or chloroacetic acid capping of the N-terminus.

### Preparation of (S)-2-((((9H-fluoren-9-yl)methoxy) carbonyl)-3-(1-(2-(tert-butoxy)-2-oxoethyl)-1H-indol-3-yl)propanoic acid

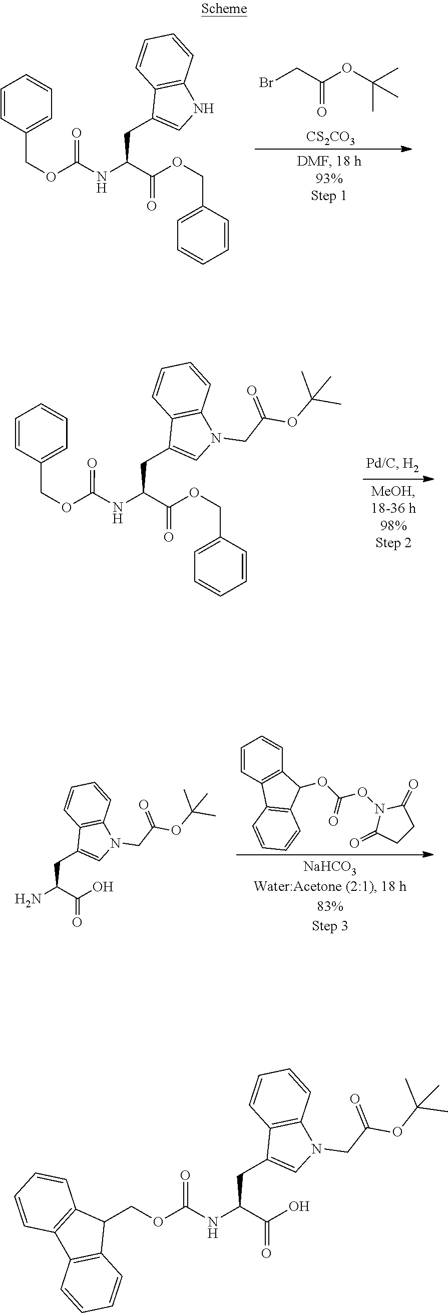

### Step 1:

To a 0° C. solution of (S)-benzyl 2-(((benzyloxy) carbonyl)amino)-3-(1H-indol-3-yl) propanoate (25.0 g, 58.3 mmol) and cesium carbonate (20.9 g, 64.2 mmol) in DMF (200 mL) was added tert-butyl 2-bromoacetate (9.36 mL, 64.2 mmol). The solution was allowed to slowly warm up to RT with stirring for 18 h. The reaction mixture was poured into ice water: aq. 1N HCl (1:1) and then extracted with EtOAc. The organic layer was washed with brine, collected, dried over MgSO₄, filtered, and then concentrated in vacuo. The resulting solid was subjected to flash chromatography (330 g column, 0-50% EtOAc: Hex over 20 column volumes) to afford(S)-benzyl 2-(((benzyloxy) carbonyl)amino)-3-(1-(2-(tert-butoxy)-2-oxoethyl)-1H-indol-3-yl) propanoate as a white solid (29.6 g, 93%).

### Step 2:

H₂ was slowly bubbled through a mixture of (S)-benzyl 2-(((benzyloxy) carbonyl)amino)-3-(1-(2-(tert-butoxy)-2-oxoethyl)-1H-indol-3-yl) propanoate (29.6 g, 54.5 mmol) and Pd—C (1.45 g, 1.36 mmol) in MeOH (200 mL) at RT for 10 min. The mixture was then stirred under positive pressure of H₂ while conversion was monitored by LCMS. After 48 h the reaction mixture was filtered through diatomaceous earth and evaporated to afford crude (S)-2-amino-3-(1-(2-(tert-butoxy)-2-oxoethyl)-1H-indol-3-yl) propanoic acid (17.0 g) which was carried into step three without additional purification.

### Step 3:

To a solution of (S)-2-amino-3-(1-(2-(tert-butoxy)-2-oxoethyl)-1H-indol-3-yl) propanoic acid (5.17 g, 16.2 mmol) and sodium bicarbonate (6.8 g, 81 mmol) in acetone: water (50.0 mL: 100 mL) was added (9H-fluoren-9-yl)methyl (2,5-dioxopyrrolidin-1-yl) carbonate (5.48 g, 16.2 mmol). The mixture stirred overnight upon which LCMS analysis indicated complete conversion. The vigorously stirred mixture was acidified via slow addition of aq 1N HCl. Once acidified, the mixture was diluted with DCM (150 mL), and the isolated organic phase was then washed with water, followed by brine. The organic layer was collected, dried over sodium sulfate, and concentrated under vacuum to afford the crude product. The crude material was purified via silica gel chromatography (330 g column, 20-80% EtOAc: Hex over 20 column 25 volumes) to afford(S)-2-((((9H-fluoren-9-yl) methoxy) carbonyl)amino)-3-(1-(2-(tertbutoxy)-2-oxoethyl)-1H-indol-3-yl) propanoic acid as a white foam (7.26 g, 83%). ¹H NMR (500 MHZ, methanol-d₄) δ 7.80 (d, J=7.6 Hz, 2H), 7.67-7.60 (m, 2H), 7.39 (t, J=7.5 Hz, 2H), 7.32-7.22 (m, 3H), 7.18 (td, J=7.6, 0.9 Hz, 1H), 7.08 (td, J=7.5, 0.9 Hz, 1H), 7.04 (s, 1H), 4.54 (dd, J=8.4, 4.9 Hz, 1H), 4.36-4.23 (m, 2H), 4.23-4.14 (m, 1H), 30 3.43-3.35 (m, 2H), 3.25-3.09 (m, 1H), 1.55-1.38 (m, 9H). ESI-MS(+) m/z=541.3 (M+H).

### Preparation of (S)-2-((((9H-fluoren-9-yl) methoxy) carbonyl)amino)-3-(4-(2-(tert-butoxy)-2-oxoethoxy)phenyl) propanoic acid

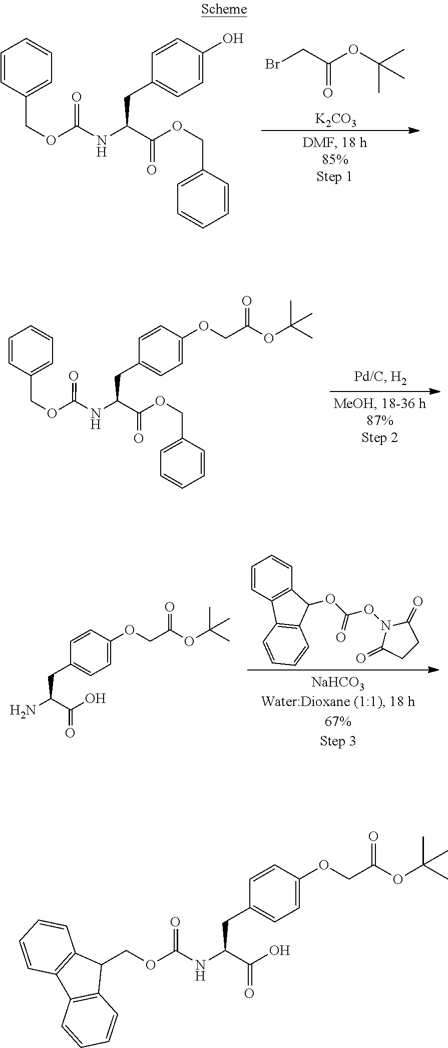

### Step 1:

To a cooled stirred solution of (S)-benzyl 2-(((benzyloxy) carbonyl)amino)-3-(4-hydroxyphenyl) propanoate (70 g, 173 mmol) and K₂CO₃ (35.8 g, 259 mmol) in DMF (350 mL) was added tert-butyl-2-bromoacetate (30.6 mL, 207 mmol) dropwise and the resulting mixture was stirred at RT overnight. The reaction mixture was diluted with 10% brine solution (1000 mL) and extracted with ethyl acetate (2×250 mL). The combined organic layer was washed with water (500 mL), saturated brine solution (500 mL), dried over anhydrous sodium sulfate, filtered, and concentrated to afford colorless gum. The crude compound was purified by flash column chromatography using 20% ethyl acetate in petroleum ether as an eluent to afford a white solid (78 g, 85%).

### Step 2:

The(S)-benzyl 2-(((benzyloxy) carbonyl)amino)-3-(4-(2-(tert-butoxy)-2-oxoethoxy)phenyl) propanoate (73 g, 140 mmol) was dissolved in MeOH (3000 mL) and purged with nitrogen for 5 min. To the above purged mixture was added Pd/C (18 g, 16.91 mmol) and stirred under hydrogen pressure of 3 kg for 15 hours. The reaction mixture was filtered through a bed of diatomaceous earth (Celite®) and washed with methanol (1000 mL). The filtrate was concentrated under vacuum to afford a white solid (36 g, 87%).

### Step 3:

To a stirred solution of (S)-2-amino-3-(4-(2-(tert-butoxy)-2-oxoethoxy)phenyl) propanoic acid (38 g, 129 mmol) and sodium bicarbonate (43.2 g, 515 mmol) in water (440 mL) was added Fmoc-OSu (43.4 g, 129 mmol) dissolved in dioxane (440 mL) dropwise and the resulting mixture was stirred at RT overnight. The reaction mixture was diluted with 1.5 N HCl (200 mL) and water (500 mL) and extracted with ethyl acetate (2×250 mL). The combined organic layer was washed with water (250 mL), saturated brine solution (250 mL), and dried over Na₂SO₄, filtered, and concentrated to afford a pale yellow gum. The crude compound was purified by column chromatography using 6% MeOH in chloroform as an eluent to afford pale green gum. The gum was further triturated with petroleum ether to afford an off-white solid (45 g, 67%). ¹H NMR (400 MHz, DMSO-d₆) δ 12.86-12.58 (m, 1H), 7.88 (d, J=7.5 Hz, 2H), 7.73-7.61 (m, 3H), 7.58-7.47 (m, 1H), 7.44-7.27 (m, 4H), 7.18 (d, J=8.5 Hz, 2H), 6.79 (d, J=8.5 Hz, 2H), 4.57 (s, 2H), 4.25-4.10 (m, 4H), 3.34 (br s, 3H), 3.02 (dd, J=13.8, 4.3 Hz, 1H), 2.81 (dd, J=14.1, 10.5 Hz, 1H), 1.41 (s, 9H).

### Preparation of (S)-2-((((9H-fluoren-9-yl) methoxy) carbonyl)amino)-3-(4-(tert-butoxycarbonyl)phenyl) propanoic acid

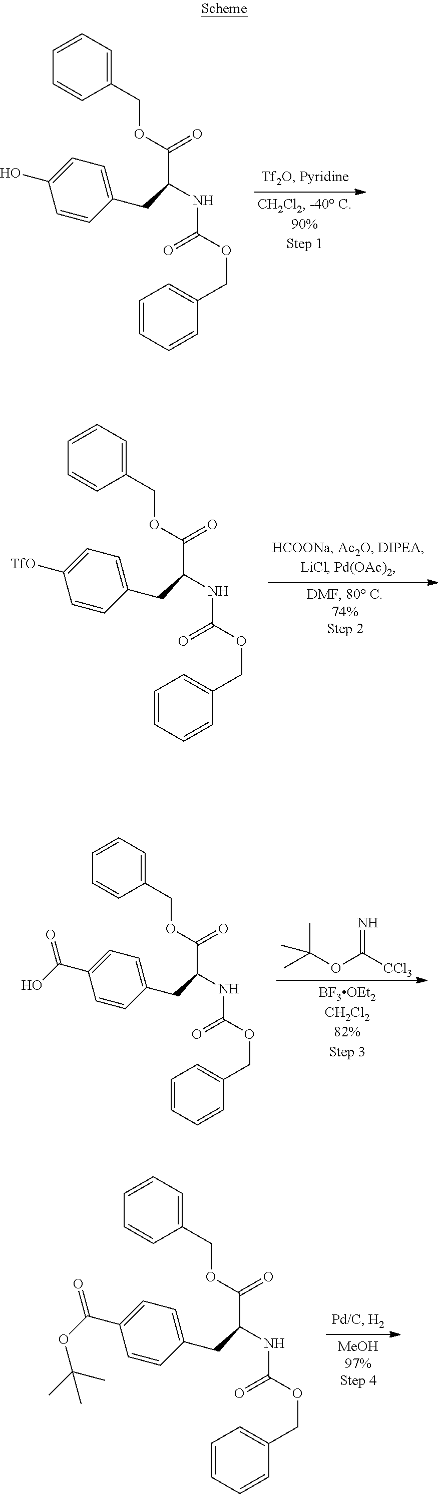

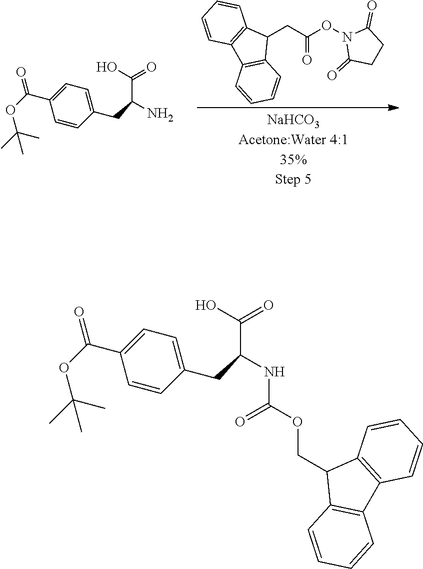 [SMILES: CC(C)(C)OC(=O)c1ccc(C[C@H](N)C(=O)O)cc1 ; O=C(CC1c2ccccc2-c2ccccc21)ON1C(=O)CCC1=O ; CC(C)(C)OC(=O)c1ccc(C[C@H](NC(=O)OCC2c3ccccc3-c3ccccc32)C(=O)O)cc1]

### Step 1.

(S)-Benzyl 2-(((benzyloxy) carbonyl)amino)-3-(4-hydroxyphenyl) propanoate (10 g, 24.66 mmol) was taken in DCM (100 mL) in a 250 mL multi-neck round bottom flask under magnetic stirring with N₂ outlet. The reaction mixture was cooled to −40° C., pyridine (5.49 mL, 67.8 mmol) was added slowly and then stirred at the same temperature for 20 minutes, followed by addition of triflic anhydride (11.46 mL, 67.8 mmol) slowly at −40° C. and allowed to stir at −40° C. for 2 hours. The reaction mixture was quenched with water at −10° C., and then added citric acid solution (50 mL). The organic layer was extracted in DCM, and the separated organic layer was dried over anhydrous Na₂SO₄, filtered, and then evaporated to give(S)-benzyl 2-(((benzyloxy) carbonyl)amino)-3-(4-(((trifluoromethyl) sulfonyl)oxy)phenyl) propanoate (11.93 g, 22.20 mmol, 90% yield) as a pale yellow solid.

### Step 2.

A solution of DMF (1500 mL) was purged with nitrogen for 10 min. To this was added sodium formate (114 g, 1676 mmol) and acetic anhydride (106 mL, 1123 mmol). Purging continued and the mixture was cooled to 0° C. DIPEA (194 mL, 1111 mmol) was added and the reaction mixture was allowed to stir for 1 h at RT under nitrogen atmosphere.

To a 10-liter autoclave was added DMF (3200 mL) and the system was purged with nitrogen. Under the nitrogen purging conditions, (S)-benzyl 2-(((benzyloxy) carbonyl)amino)-3-(4-(((trifluoromethyl) sulfonyl)oxy)phenyl) propanoate (300 g, 558 mmol), lithium chloride (71 g, 1675 mmol), 1,3-bis(diphenylphosphino) propane (24.17 g, 58.6 mmol) were added followed by the addition of palladium (II) acetate (12.9 g, 57.5 mmol). To this reaction mixture was added the above prepared solution and heated to 80° C. for 16 h.

The reaction mass was diluted with ethyl acetate and water. The phases were separated and the ethyl acetate layer was washed with water and brine solution, dried over anhydrous sodium sulphate, filtered, and concentrated. The crude material was added to a torrent column and was eluted with petroleum ether and ethyl acetate. The fractions at 30%-65% ethyl acetate in petroleum ether were concentrated to afford a cream solid (300 g), which was dissolved in ethyl acetate (700 mL) and petroleum ether was added slowly. At about 20% ethyl acetate in petroleum ether a white solid precipitated out, which was filtered and washed with 20% ethyl acetate in petroleum ether to obtain a white solid (180 g, yield 74%). Step 3.

To a 2000-ml multi-neck round-bottomed flask was charged(S)-4-(3-(benzyloxy)-2-(((benzyloxy) carbonyl)amino)-3-oxopropyl)benzoic acid (130 g, 300 mmol), dichloromethane (260 mL) and cyclohexane (130 mL). To the slurry reaction mixture was added BF₃·OEt₂ (3.80 mL, 30.0 mmol) at room temperature, followed by the addition of tert-butyl 2,2,2-trichloroacetimidate (262 g, 1200 mmol) slowly at room temperature over 30 min. Upon addition, the slurry slowly started dissolving and at the end of the addition it was completely dissolved. The reaction mixture was allowed to stir at room temperature for 16 h. The reaction mixture was diluted with DCM and the remaining solids were removed by filtration. The filtrate was concentrated and purified by flash chromatography. The crude material was purified by Torrent using 1.5 Kg silicycle column. The product spot was eluted at 15% ethyl acetate/petroleum ether mixture. The collected fractions were concentrated to obtain a colorless liquid (120 g, yield 82%).

### Step 4.

(S)-tert-Butyl 4-(3-(benzyloxy)-2-(((benzyloxy) carbonyl)amino)-3-oxopropyl)benzoate (200 g, 409 mmol) was dissolved in MeOH (4000 mL) and N₂ was purged for 10 min. Pd/C (27.4 g, 25.7 mmol) was added. The reaction was shaken under H₂ for 16 h at room temperature. The reaction mass was filtered through celite bed and the bed was washed with methanol. The obtained filtrate was concentrated to obtain a pale yellow solid. The obtained solid was stirred with 5% methanol: diethyl ether mixture for 15 min before being filtered, dried under vacuum to obtain a pale yellow solid. It was made slurry with 5% methanol in diethyl ether and stirred for 15 min, filtered, and dried to give(S)-2-amino-3-(4-(tert-butoxycarbonyl)phenyl) propanoic acid as a white solid (105 g, yield 97%). Analysis condition E: Retention time=0.971 min; ESI-MS(+) m/z [M+H]⁺: 266.2.

### Step 5.

(S)-2-Amino-3-(4-(tert-butoxycarbonyl)phenyl) propanoic acid (122 g, 460 mmol) was dissolved in acetone (1000 mL) and then water (260 mL) and sodium bicarbonate (116 g, 1380 mmol) were added. It was cooled to 0° C. and Fmoc-OSu (155 g, 460 mmol) was added portionwise into the reaction mixture. After completion of addition it was stirred at room temperature for 16 h. The reaction mixture was diluted with dichloromethane (2 L) and then water was added (1.5 L). The organic layer was washed with saturated citric acid solution and extracted, and the aqueous layer was again extracted with DCM. The combined organic layer was washed with 10% citric acid solution, brine solution, and dried over Na₂SO₄, and evaporated to dryness. The obtained white solid was made slurry with diethyl ether, filtered, and dried to get the desired product as a white solid (80 g, yield 35%). ¹H NMR (400 MHz, DMSO-d6) δ 7.87 (d, J=7.5 Hz, 2H), 7.83-7.73 (m, 3H), 7.60 (t, J=8.5 Hz, 2H), 7.51-7.24 (m, 7H), 4.26-4.11 (m, 4H), 3.45-3.27 (m, 4H), 3.17 (br dd, J=13.8, 4.3 Hz, 1H), 2.94 (dd, J=13.5, 11.0 Hz, 1H), 2.52-2.48 (m, 4H), 1.51 (s, 9H).

### Preparation of tert-butyl (R)-2-((((9H-fluoren-9-yl) methoxy) carbonyl)amino)-3-iodopropanoate

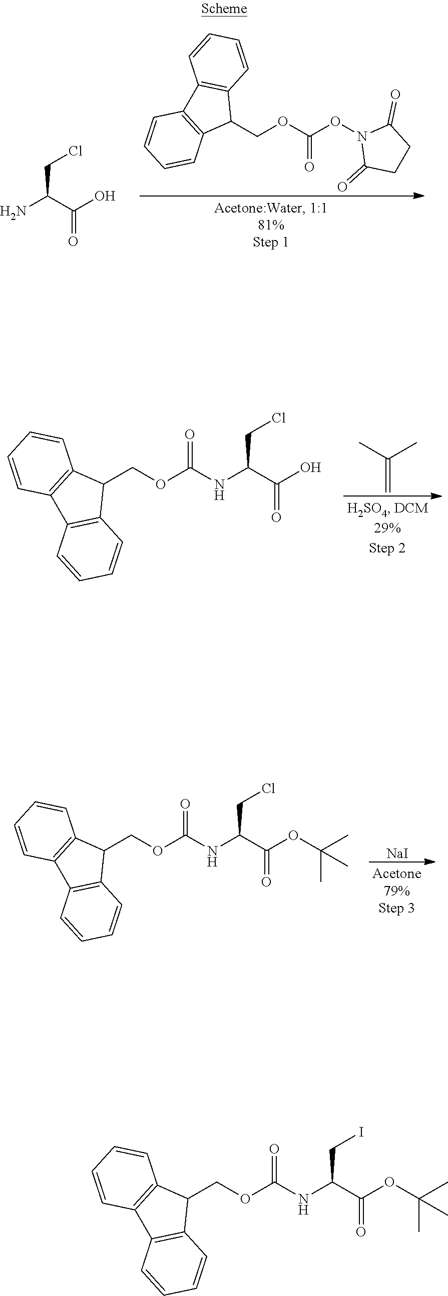

### Step 1.

To a solution of (R)-2-amino-3-chloropropanoic acid hydrochloride (125 g, 781 mmol) in a 1:1 mixture of acetone (1 L) and water (1 L) was added Na₂CO₃ (182 g, 1719 mmol) followed by Fmoc-OSu (250 g, 742 mmol). The reaction was stirred at RT overnight. It was extracted with ethyl acetate (2×500 mL) and the aq. layer was acidified with 5N HCl. The HCl solution was extracted with ethyl acetate (1500 mL, then 2×500 mL). The combined organic layers were dried over anhydrous MgSO₄, filtered, and concentrated to give the crude product (R)-2-((((9H-fluoren-9-yl) methoxy) carbonyl)amino)-3-chloropropanoic acid. The product (220 g) was taken to the next step as such.

### Step 2.

A solution of (R)-2-((((9H-fluoren-9-yl) methoxy) carbonyl)amino)-3-chloropropanoic acid (220 g, 636 mmol) in DCM (2 L) was cooled to −20° C. 2-Methylpropene (200 mL, 636 mmol) was bubbled into the solution for 15 mins, then H₂SO₄ (57.7 mL, 1082 mmol) was added and the mixture was stirred at RT overnight. To the reaction mixture was added water (500 mL). The layers were separated and the aqueous layer was extracted DCM (2×500 mL). The combined organic layers were dried over anhydrous MgSO₄, filtered, and evaporated. The crude was purified by flash chromatography using petroleum ether and ethyl acetate elution solvents. The desired fractions were combined and concentrated to give the product (R)-tert-butyl 2-((((9H-fluoren-9-yl) methoxy) carbonyl)amino)-3-chloropropanoate (83 g, 182 mmol, 29% yield).

### Step 3.

To a solution of (R)-tert-butyl 2-((((9H-fluoren-9-yl) methoxy) carbonyl)amino)-3-chloropropanoate (80 g, 199 mmol) in acetone (1000 mL) was added sodium iodide (119 g, 796 mmol) and the reaction was heated to reflux for 40 hours. Acetone was removed by rotavap and the crude product was diluted with water (1000 mL) and DCM (1000 mL). The layers were separated and the organic layer was washed with aqueous saturated sodium sulphite solution (1000 mL) and brine (1000 mL). The organic layer was dried over anhydrous Na₂SO₄, filtered, and concentrated. The crude was purified by flash chromatography using 7 to 9% of ethyl acetate in petroleum ether. The desired product fractions were combined and concentrated to afford the product (R)-tert-butyl 2-((((9H-fluoren-9-yl) methoxy) carbonyl)amino)-3-iodopropanoate (83 g, 156 mmol, 79%). ¹H NMR (400 MHz, CDCl₃) δ 7.77 (d, J=7.5 Hz, 2H), 7.62 (d, J=7.5 Hz, 2H), 7.45-7.30 (m, 4H), 5.67 (br d, J=7.0 Hz, 1H), 4.54-4.32 (m, 3H), 4.30-4.21 (m, 1H), 3.71-3.50 (m, 2H), 1.56-1.48 (m, 9H).

### Preparation of (S)-2-((((9H-fluoren-9-yl) methoxy) carbonyl)amino)-3-(2-methyl-1H-indol-3-yl) propanoic acid

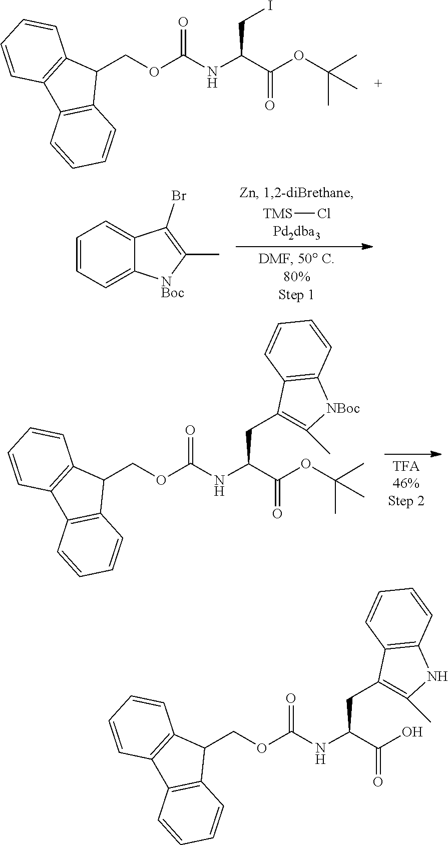 [SMILES: CC(C)(C)OC(=O)[C@H](CI)NC(=O)OCC1c2ccccc2-c2ccccc21 ; Cc1c(Br)c2ccccc2n1C(=O)OC(C)(C)C ; C[Si](C)(C)Cl ; Cc1c(C[C@H](NC(=O)OCC2c3ccccc3-c3ccccc32)C(=O)OC(C)(C)C)c2ccccc2n1C(=O)OC(C)(C)C ; Cc1[nH]c2ccccc2c1C[C@H](NC(=O)OCC1c2ccccc2-c2ccccc21)C(=O)O]

### Step 1.

In a 100-ml three-neck, flame-dried, nitrogen-purged round-bottomed flask, zinc (2.319 g, 35.5 mmol) was added under argon atmosphere and the flask was heated to 150° C. using a hot gun and was purged with argon. To the reaction flask, DMF (50 mL) was added followed by the addition of 1,2-dibromoethane (0.017 mL, 0.20 mmol) and TMS-CI (0.026 mL, 0.20 mmol) under argon atmosphere and then stirred for 10 min. To the reaction mixture (R)-tert-butyl 2-((((9H-fluoren-9-yl) methoxy) carbonyl)amino)-3-iodopropanoate (5 g, 10.14 mmol) was added and the reaction was stirred for 1 h. The reaction progress was monitored via TLC and LCMS, till the starting iodide was completely converted into the Zn-complex. The solution of organozinc reagent was allowed to cool to room temperature and then tris(dibenzylideneacetone) dipalladium (0) (Pd₂(dba)₃) (0.23 g, 0.25 mmol), dicyclohexyl(2′,6′-dimethoxy-[1,1′-biphenyl]-2-yl)phosphine (SPhos) (0.21 g, 0.51 mmol), and tert-butyl 3-bromo-2-methyl-1H-indole-1-carboxylate (3.77 g, 12.16 mmol) were added. The reaction mixture was allowed to stir at RT under a positive pressure of nitrogen for 1 h and then heated to 50° C. for 6 hrs. The reaction progress was monitored via LCMS. The mixture was diluted with EtOAc (700 mL) and filtered through Celite. The organic phase was washed with sat. NH₄Cl (250 mL), water (2×200 mL), and sat. NaCl (aq) (250 mL), dried over anhydrous Na₂SO₄ (s), concentrated, and dried under vacuum to afford the crude compound (19 g). It was purified through ISCO flash chromatography using 330 g redisep column and the product was eluted with 7 to 9% of ethyl acetate in petroleum ether. The above reaction and purification were repeated. The pure fractions were concentrated to give tert-butyl(S)-3-(2-((((9H-fluoren-9-yl) methoxy) carbonyl)amino)-3-(tert-butoxy)-3-oxopropyl)-2-methyl-1H-indole-1-carboxylate as a brownish solid (10.2 g. 95% pure, ca. 80% yield). Analysis condition G: Retention time=4.23 min; ESI-MS(+) m/z [M+2H][M-Boc-tBu+H]⁺: 441.2.

### Step 2.

In a 25-ml multi neck, round-bottomed flask, DCM (65 mL) was added followed by(S)-tert-butyl 3-(2-((((9H-fluoren-9-yl) methoxy) carbonyl)amino)-3-(tert-butoxy)-3-oxopropyl)-2-methyl-1H-indole-1-carboxylate (6.5 g, 10.89 mmol) under nitrogen atmosphere at RT. The reaction mixture was cooled to 0° C., triethylsilane (4.18 mL, 26.1 mmol) was added followed by the addition of TFA (5.87 mL, 76 mmol) dropwise at 0° C. The temperature of the reaction mixture was slowly brought to RT and stirred at RT for 4 h. The reaction progress was monitored by TLC. To the reaction mixture, TFA (5.87 mL, 76 mmol) was added. The reaction mixture was stirred at RT overnight, and concentrated under reduced pressure. The crude material was triturated with hexanes and stored in cold room to give a brown colored solid (crude weight: 6.5 g). It was purified via reverse phase flash chromatography, and the pure fractions were concentrated to obtain the desired final product as an off-white powder (2.3 g, 46%). 1H NMR (DMSO-d₆): δ ppm: 10.65 (s, 1H), 7.84 (d, J=9.12 Hz, 2H), 7.65 (d, J=9.12 Hz, 2H), 7.42-7.49 (m, 1H), 7.30-7.38 (m, 2H), 7.26-7.29 (m, 2H), 7.17-7.19 (m, 2H), 6.91-6.95 (m, 1H), 6.85-6.88 (t, J=7.85 Hz, 1H), 4-16-4.18 (m, 2H), 4.01-4.06 (m, 1H), 3.09-3.14 (m, 1H), 2.96-2.99 (m, 1H), 2.50 (s, 3H). Analysis condition F: Retention time=1.37 min; ESI-MS(+) m/z [M+2H][M+H]⁺: 441.2.

### Preparation of (S)-2-((((9H-fluoren-9-yl) methoxy) carbonyl)amino)-3-(7-methyl-1H-indol-3-yl) propanoic acid

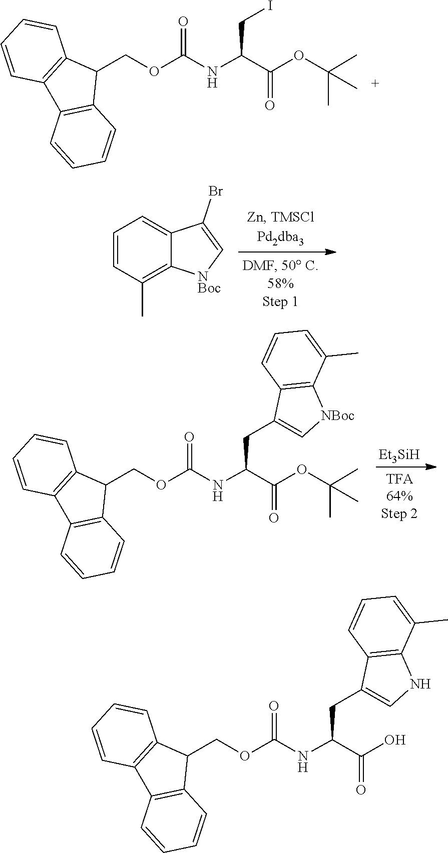

### Step 1.

In a 50-ml round-bottomed flask, dry zinc (0.928 g, 14.19 mmol) was charged and flushed with argon three times and then the flask was heated to 150° C. for 5 min and then allowed to cool to room temperature and flushed with argon 3 times. DMF (20 mL) was added followed by the addition of 1,2-dibromoethane (6.99 μl, 0.081 mmol) and TMS-Cl (0.013 mL, 0.10 mmol). Successful zinc insertion was accompanied by a noticeable exotherm. After 5 min (R)-tert-butyl 2-((((9H-fluoren-9-yl) methoxy) carbonyl)amino)-3-iodopropanoate (2.0 g, 4.05 mmol) was added and the reaction was stirred for 30 min. In a 50-ml round-bottomed flask equipped charged with Argon was added the above alkyl zinc reagent, tert-butyl 3-bromo-7-methyl-1H-indole-1-carboxylate (1.26 g, 4.05 mmol) followed by 2-dicyclohexylphosphino-2′,6′-dimethoxybiphenyl (SPhos) (0.083 g, 0.20 mmol) and Pd₂ (dba) 3 (0.093 g, 0.101 mmol). After the addition the reaction mixture was heated to 50° C. overnight. Another equivalents of Sphos and Pd₂ (dba) 3 was added and heating continued for another 16 h. The reaction mixture was diluted with EtOAc (100 mL) and filtered through Celite. The organic phase was washed with sat. aq. NH₄Cl (100 mL), water (50 mL), and sat NaCl (100 mL), dried over anhydrous Na₂SO₄ (s), concentrated, and dried under vacuum. After purification by flash chromatography the desired tert-butyl(S)-3-(2-((((9H-fluoren-9-yl) methoxy) carbonyl)amino)-3-(tert-butoxy)-3-oxopropyl)-2-methyl-1H-indole-1-carboxylate was obtained in 58% yield.

### Step 2.

Final product was obtained following the same procedure of (S)-2-((((9H-fluoren-9-yl) methoxy) carbonyl)amino)-3-(2-methyl-1H-indol-3-yl) propanoic acid. TFA hydrolysis with triethylsilane afforded the desired(S)-2-((((9H-fluoren-9-yl) methoxy) carbonyl)amino)-3-(7-methyl-1H-indol-3-yl) propanoic acid as an off white solid in 64% yield after purification by reverse phase flash chromatography. Analysis condition E: Retention time=2.16 min; ESI-MS(+) m/z [M+H]⁺: 441.1. ¹H NMR (300 MHz, DMSO-d6) Shift 12.70 (br s, 1H), 10.81 (br s, 1H), 7.88 (d, J=7.6 Hz, 2H), 7.76-7.56 (m, 2H), 7.49-7.21 (m, 5H), 7.17 (d, J=2.3 Hz, 1H), 6.94-6.84 (m, 2H), 4.29-4.13 (m, 3H), 4.07 (br s, 1H), 3.19 (br dd, J=14.7, 4.5 Hz, 1H), 3.01 (br dd, J=14.5, 9.6 Hz, 1H), 2.47-2.40 (m, 3H), 0.02-−0.06 (m, 1H).

### Preparation of (S)-2-((((9H-fluoren-9-yl) methoxy) carbonyl)amino)-3-(quinolin-6-yl) propanoic acid

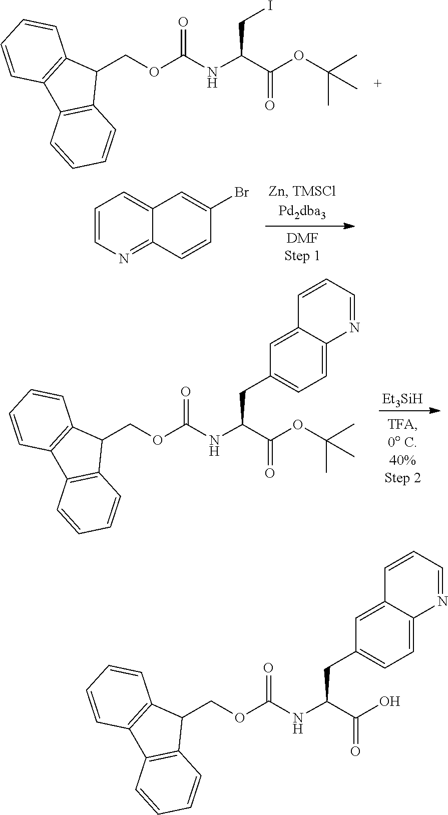

### Step 1.

In a 25-ml round bottom flask, dry zinc (2.32 g, 35.5 mmol) was charged and argon was flashed three times. The flask was heated to 150° C. for 5 min and then allowed to cool to room temp and flushed with argon 3 times. DMF (50 mL) was added followed by the addition of 1,2-dibromoethane (0.017 mL, 0.20 mmol) and TMS-Cl (0.032 mL, 0.25 mmol). Successful zinc insertion was accompanied by a noticeable exotherm. After 5 min (R)-tert-butyl 2-((((9H-fluoren-9-yl) methoxy) carbonyl)amino)-3-iodopropanoate (5.0 g, 10.14 mmol) was added and the reaction was stirred for 30 min.

In a 250-ml round bottom flask purged with Argon was added DMF (50 mL), 6-bromoquinoline (2.53 g, 12.16 mmol), previously prepared solution of alkyl zinc reagent, (R)-tert-butyl 2-((((9H-fluoren-9-yl) methoxy) carbonyl)amino)-3-iodopropanoate (5.0 g, 10.14 mmol) followed by 2-dicyclohexylphosphino-2′,6′-diisopropoxy-1,1′-biphenyl (RuPhos) (0.24 g, 0.51 mmol) and Pd₂(dba)₃ (0.23 g, 0.25 mmol). The reaction mixture was allowed to stir at rt for 5 h and then heated to 50° C. for 16 h. It was cooled to rt and filtered over celite and rinsed with ethyl acetate. The solution was concentrated on rotovap. Purification by flash chromatography gave the desired compound as a thick brown liquid in quantitative yields. Analysis condition E: Retention time=3.47 min; ESI-MS(+) m/z [M+H]⁺: 495.2.

### Step 2.

The final product was obtained following the same procedure of (S)-2-((((9H-fluoren-9-yl) methoxy) carbonyl)amino)-3-(2-methyl-1H-indol-3-yl) propanoic acid. TFA hydrolysis with triethylsilane afforded the desired(S)-2-((((9H-fluoren-9-yl) methoxy) carbonyl)amino)-3-(quinolin-6-yl) propanoic acid as a beige solid in 40% yield after solid-liquid extraction with diethyl ether and water. ¹H NMR (300 MHz, DMSO-d₆) δ 8.94 (br d, J=4.5 Hz, 1H), 8.49 (d, J=8.7 Hz, 1H), 8.01-7.92 (m, 2H), 7.85-7.79 (m, 3H), 7.65 (dd, J=8.3, 4.5 Hz, 1H), 7.55 (dd, J=7.2, 4.2 Hz, 2H), 7.36 (t, J=7.4 Hz, 2H), 7.26-7.14 (m, 2H), 4.32 (dd, J=10.6, 4.5 Hz, 1H), 4.18-4.08 (m, 3H), 3.38-3.29 (m, 2H), 3.11 (br d, J=10.6 Hz, 1H), 2.72 (s, 1H), 1.07 (t, J=7.0 Hz, 1H), −0.02 (s, 1H). Analysis condition E: Retention time=1.54 min; ESI-MS(+) m/z [M+H]⁺: 439.0.

### Preparation of (S)-2-((((9H-fluoren-9-yl) methoxy) carbonyl)amino)-3-(isoquinolin-6-yl) propanoic acid

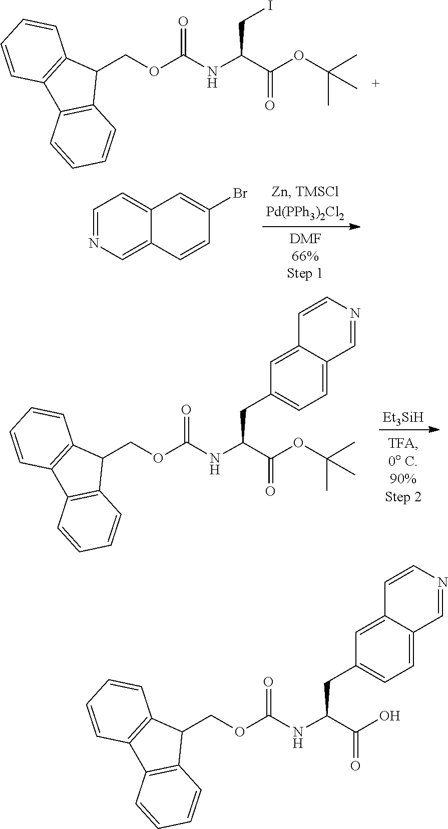

### Step 1.

In a 50-ml three neck flame-dried round bottom flask zinc (1.392 g, 21.28 mmol) was added under argon atmosphere and the flask was heated to 150° C. using a hot gun and was purged with argon. To the reaction DMF (30 mL) was added followed by the addition of 1,2-dibromoethane (10.48 μl, 0.12 mmol) and TMS-Cl (0.016 mL, 0.12 mmol) under argon. The reaction was stirred for 10 minutes. To the reaction mixture (R)-tert-butyl 2-((((9H-fluoren-9-yl) methoxy) carbonyl)amino)-3-iodopropanoate (3.0 g, 6.08 mmol) was added and the reaction was stirred for 1 hr To the reaction mixture 6-bromoisoquinoline (1.52 g, 7.30 mmol) and bis-(triphenylphosphino)-palladous chloride (0.20 g, 0.30 mmol) were added and the reaction was stirred for 16 h. The reaction mixture was diluted with ethyl acetate (50 mL), filtered through celite and washed with ethyl acetate (50 mL). The filtrate was concentrated under reduced pressure to afford the crude product as a red thick gum. The crude was purified by flash chromatography using 40 to 42% EtOAc in petroleum ether. After concentration on rotovap tert-butyl(S)-2-((((9H-fluoren-9-yl) methoxy) carbonyl)amino)-3-(isoquinolin-6-yl) propanoate (2.0 g, 66%) was obtained as a yellow gum. Analysis condition B: Retention time=2.46 min; ESI-MS(+) m/z [M+H]⁺: 495.3.

### Step 2.

The final product was obtained following the same procedure of (S)-2-((((9H-fluoren-9-yl) methoxy) carbonyl)amino)-3-(2-methyl-1H-indol-3-yl) propanoic acid. TFA hydrolysis with triethylsilane afforded the desired(S)-2-((((9H-fluoren-9-yl) methoxy) carbonyl)amino)-3-(isoquinolin-6-yl) propanoic acid as a grey solid in 90% yield after recrystallization in EtOAc and hexanes. ¹H NMR (400 MHz, METHANOL-d₄) δ 9.55 (s, 1H), 8.46 (d, J=6.5 Hz, 1H), 8.33 (d, J=8.5 Hz, 1H), 8.17 (d, J=6.0 Hz, 1H), 8.08 (s, 1H), 7.99-7.86 (m, 1H), 7.78 (dd, J=7.5, 4.0 Hz, 2H), 7.66-7.48 (m, 2H), 7.43-7.30 (m, 2H), 7.30-7.17 (m, 2H), 4.68 (dd, J=10.0, 4.5 Hz, 1H), 4.32-4.13 (m, 2H), 4.12-3.84 (m, 1H), 3.61 (dd, J=13.8, 4.8 Hz, 1H), 3.32-3.26 (m, 1H), 1.46 (s, 1H). Analysis condition B: Retention time=2.77 min; ESI-MS(+) m/z [M+H]⁺: 439.2.

### Preparation of (S)-2-((((9H-fluoren-9-yl) methoxy) carbonyl)amino)-3-(isoquinolin-4-yl) propanoic acid

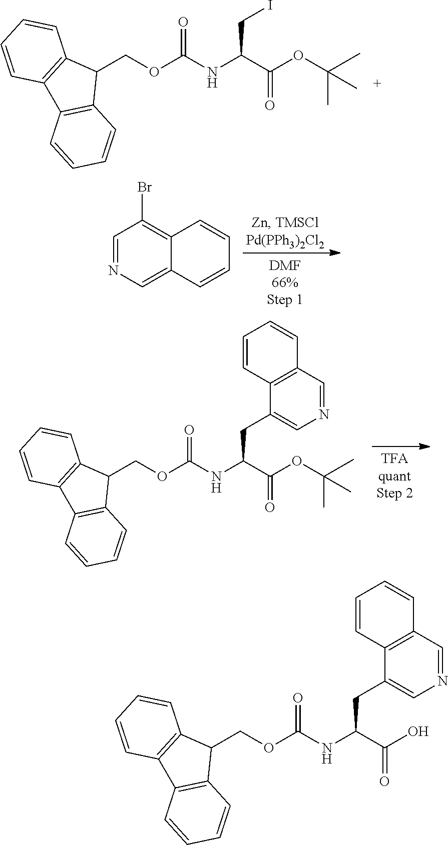

### Step 1.

To a stirred mixture of zinc (2.319 g, 35.5 mmol) in DMF (50 mL) was added dibromomethane (0.071 mL, 1.014 mmol) and TMS-Cl (0.130 mL, 1.014 mmol). Exotherm was observed. The reaction mixture was for 10 min. (R)-tert-butyl 2-((((9H-fluoren-9-yl) methoxy) carbonyl)amino)-3-iodopropanoate (5 g, 10.14 mmol) was added and again exotherm was observed. The reaction was allowed to stir for 1 h at room temperature. 2-Dicyclohexylphosphino-2′,6′-dimethoxybiphenyl (0.21 g, 0.51 mmol), tris(dibenzylideneacetone) dipalladium (0) (0.23 g, 0.25 mmol) and 4-bromoisoquinoline (2.11 g, 10.14 mmol) were added sequentially and the reaction was heated to 50° C. for 16 h. The reaction mixture was cooled to rt and treated with saturated ammonium chloride solution (200 mL). The crude was diluted with the ethyl acetate (300 mL). Layers were separated and the organic layer was washed with brine and dried over anhydrous sodium sulphate. After filtration and concentration the crude product was purified by flash chromatography eluting with 30% of ethyl acetate in petroleum ether to afford tert-butyl(S)-2-((((9H-fluoren-9-yl) methoxy) carbonyl)amino)-3-(isoquinolin-4-yl) propanoate (2.5 g, 50%).

Analysis condition E: Retention time=3.44 min; ESI-MS(+) m/z [M+H]⁺: 495.2.

### Step 2.

The final product was obtained following the same procedure of (S)-2-((((9H-fluoren-9-yl) methoxy) carbonyl)amino)-3-(2-methyl-1H-indol-3-yl) propanoic acid. TFA hydrolysis afforded the desired(S)-2-((((9H-fluoren-9-yl) methoxy) carbonyl)amino)-3-(isoquinolin-4-yl) propanoic acid as an off white solid in quantitative yield after purification diethyl ether trituration. ¹H NMR (400 MHz, DMSO-d₆) δ 9.55 (s, 1H), 8.52 (s, 1H), 8.44-8.24 (m, 2H), 8.18-8.00 (m, 1H), 7.95-7.80 (m, 4H), 7.59 (br d, J=7.5 Hz, 1H), 7.56 (br d, J=7.5 Hz, 1H), 7.47-7.34 (m, 2H), 7.34-7.24 (m, 2H), 4.46-4.30 (m, 1H), 4.25-4.02 (m, 3H), 3.69 (dd, J=14.1, 4.5 Hz, 1H), 3.37 (dd, J=14.1, 10.5 Hz, 1H), 0.10-0.11 (m, 1H). Analysis condition E: Retention time=1.57 min; ESI-MS(+) m/z [M+H]⁺: 441.2.

### Preparation of (S)-2-((((9H-fluoren-9-yl) methoxy) carbonyl)amino)-3-(4-(tert-butoxy)-3,5-difluorophenyl) propanoic acid

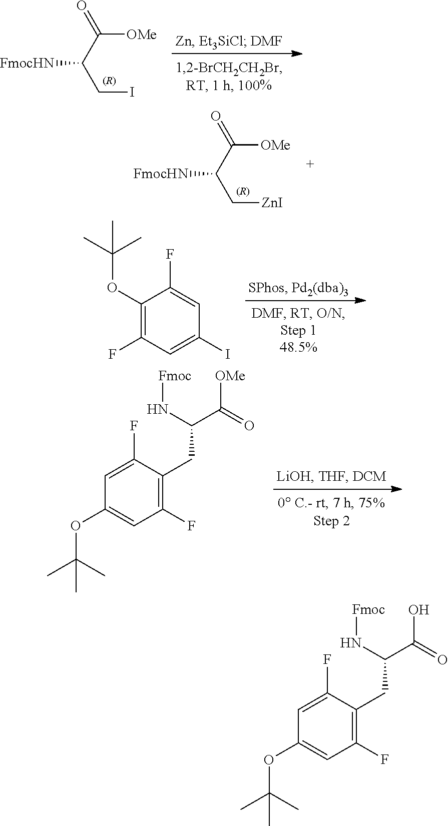

### Step 1.

The compound was prepared following the same procedure of tert-butyl(S)-2-((((9H-fluoren-9-yl) methoxy) carbonyl)amino)-3-(isoquinolin-4-yl) propanoate. First Negishi coupling with methyl (R)-2-((((9H-fluoren-9-yl) methoxy) carbonyl)amino)-3-iodopropanoate at 50° C. afforded the desired methyl(S)-2-((((9H-fluoren-9-yl) methoxy) carbonyl)amino)-3-(4-(tert-butoxy)-2,6-difluorophenyl) propanoate (5.5 g, 48.5% yield) after purification by flash chromatography.

Analysis condition E: Retention time=3.99 min; ESI-MS(+) m/z [M+NH₄]⁺: 527.2.

### Step 2.

In a multi-neck round bottom flask methyl(S)-2-((((9H-fluoren-9-yl) methoxy) carbonyl)amino)-3-(4-(tert-butoxy)-3,5-difluorophenyl) propanoate (11 g, 21.59 mmol) was added followed by the addition of tetrahydrofuran (132 mL) under nitrogen atmosphere at RT. The reaction mixture was cooled to 0° C. and LiOH (1.09 g, 45.3 mmol) in water (132 mL) solution was added. The reaction was stirred for 3 h. It was concentrated under reduced pressure below 38° C. to remove the solvent. The crude compound was cooled to 0° C., sat. Citric acid solution was added to adjust the pH to 4-5. It was extracted with ethyl acetate (3×250 mL). The combined organic layer was washed with water (200 mL) followed by brine (200 mL). The organic layer dried over sodium sulphate, filtered and concentrated under reduced pressure to give the crude (12 g) as a colorless thick mass. The crude compound was purified through ISCO using 120 g redisep column, the product was eluted with 20% of ethyl acetate in petroleum ether. The reactions were concentrated to give (S)-2-((((9H-fluoren-9-yl) methoxy) carbonyl)amino)-3-(4-(tert-butoxy)-3,5-difluorophenyl) propanoic acid (9.0 g, 82%, HPLC purity 97%) as a white fluffy solid. Analysis condition E: Retention time=3.62 min; ESI-MS(+) m/z [M+H]⁺: 513.2. ¹H NMR (CDCl3, 400 MHz) d 7.75 (d, J=7.6 Hz, 2H), 7.60 (m, 2H), 7.39 (t, J=7.6 Hz, 2H), 7.30 (m, 2H), 6.71 (d, J=7.6 Hz, 2H), 5.26 (m, 1H), 4.65 (m, 1H), 4.48-4.38 (m, 2H), 4.20 (m, 1H), 3.14-2.99 (m, 1H), 1.35 (s, 9H).

### Preparation of (S)-2-((((9H-fluoren-9-yl) methoxy) carbonyl)amino)-3-(isoquinolin-8-yl) propanoic acid

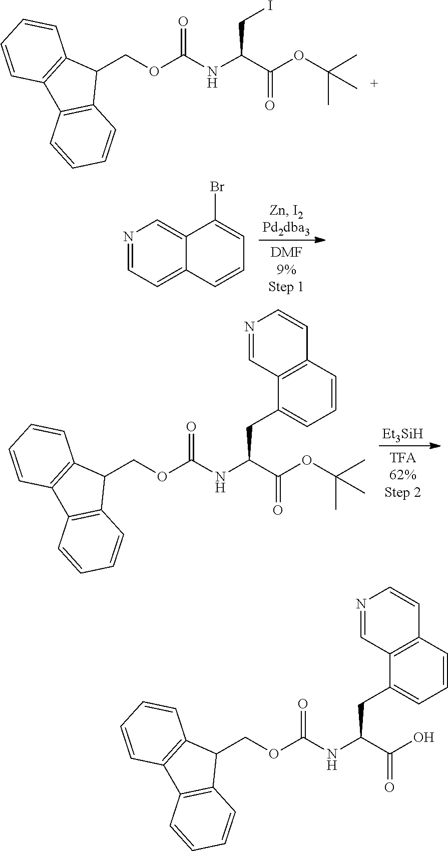

### Step 1.

Zinc (0.79 g, 12.00 mmol) was added to a flame-dried, nitrogen-purged side arm round-bottomed flask. DMF (5 mL) was added via syringe, followed by a catalytic amount of iodine (0.16 g, 0.63 mmol). A color change of the DMF was observed from colorless to yellow and back again. Protected (R)-tert-butyl 2-((((9H-fluoren-9-yl) methoxy) carbonyl)amino)-3-iodopropanoate (1.97 g, 4.00 mmol) was added immediately, followed by a catalytic amount of iodine (0.16 g, 0.63 mmol). The solution was stirred at room temperature; successful zinc insertion was accompanied by a noticeable exotherm. The solution of organozinc reagent was allowed to cool to room temperature and then Pd₂(dba)₃ (0.088 g, 0.096 mmol), dicyclohexyl(2′,6′-dimethoxy-[1,1′-biphenyl]-2-yl)phosphine (0.082 g, 0.200 mmol) and 8-bromoisoquinoline (1.082 g, 5.20 mmol) were added sequentially. The reaction mixture was stirred at 50 C for 4 h. under a positive pressure of nitrogen. The reaction mixture was cooled to rt, diluted with EtOAc (200 mL) and passed through Celite. The organic solvent was washed with sat. aq. NH₄Cl (200 mL), water (150 mL), and sat. aq. NaCl (200 mL), dried over Na₂SO₄, concentrated, and dried under vacuum to afford the crude compound. It was purified using ISCO combiflash column chromatography (24 g silica gel column, hexanes/ethyl acetate as the eluents) to afford(S)-tert-butyl 2-((((9H-fluoren-9-yl) methoxy) carbonyl)amino)-3-(isoquinolin-8-yl) propanoate (380 mg, 0.768 mmol, 19.21% yield). Analysis condition G: Retention time=2.59 min; ESI-MS(+) m/z [M+H]⁺: 495.3.

### Step 2.

(S)-tert-Butyl 2-((((9H-fluoren-9-yl) methoxy) carbonyl)amino)-3-(isoquinolin-8-yl) propanoate (380 mg, 0.768 mmol) was placed in 50-ml round bottom flask and was dissolved in DCM (8 mL). Triethylsilane (0.31 mL, 1.92 mmol) was added followed by trifluoroacetic acid (2.66 mL, 34.6 mmol). The reaction mixture was stirred at room temperature for 5 h. The solvents were evaporated, and the residue was dissolved in diethyl ether. The product was precipitated by the addition of petroleum ether. The resulting powder was then triturated with petroleum ether to yield (S)-2-((((9H-fluoren-9-yl) methoxy) carbonyl)amino)-3-(isoquinolin-8-yl) propanoic acid (320 mg, 0.712 mmol, 93% yield) as an off white solid. 1H-NMR: (400 MHz, DMSO-d6) δ ppm: 12.98 (bs, 1H), 9.79 (s, 1H), 8.62 (d, J=9.42 Hz, 1H), 8.22 (d, J=9.42 Hz, 1H), 8.06 (d, J=9.42 Hz, 1H), 7.84-7.93 (m, 4H), 7.74-7.76 (m, 1H), 7.56-7.58 (m, 1H), 7.38-7.42 (m, 2H), (m, 3H), 7.26-7.30 (m, 2H), 4.41 (m, 1H), 4.10-4.15 (m, 3H), 3.731-3.66 (m, 1H), 3.47-3.50 (m, 1H). Analysis condition G: Retention time=2.012 min; ESI-MS(+) m/z [M+H]⁺: 439.2 with 97.5% purity.

### Preparation of (S)-2-((((9H-fluoren-9-yl) methoxy) carbonyl)amino)-3-(7-fluoro-1H-indol-3-yl)propanoic acid

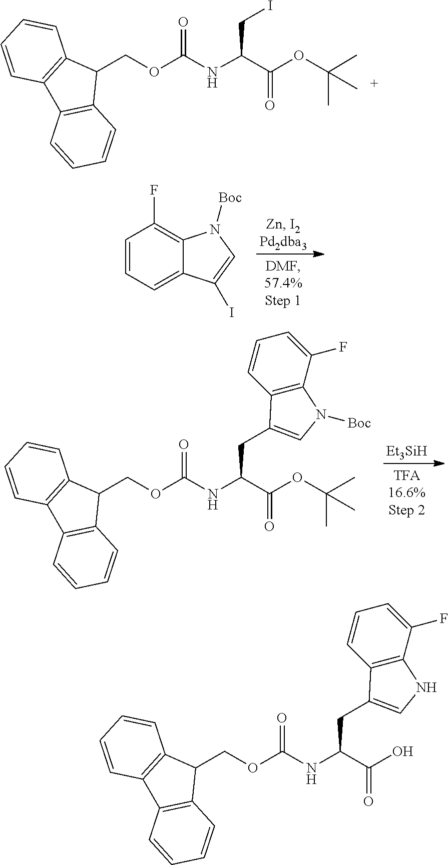

Step 1. Synthesis of tert-butyl 6-fluoro-3-iodo-1H-indole-1-carboxylate from 6-fluoro-1H-indole: A solution of iodine (3.76 g, 14.80 mmol) in DMF (15 mL) was dropped to the solution of 6-fluoro-1H-indole (2 g, 14.80 mmol) and potassium hydroxide (2.076 g, 37.0 mmol) in DMF (15 mL) at room temperature and the mixture was stirred for 45 min. The reaction mixture was then poured on 200 mL of ice water containing 0.5% ammonia and 0.1% sodium disulfite. The mixture was placed in a refrigerator to ensure the complete precipitation. The precipitate was filtered, washed with 100 mL ice water and dried in vacuo to obtain 3.80 g. The solid was suspended in dichloromethane (25 mL). 4-Dimethylaminopyridine (160 mg, 10 mol %) and di-tert-butyl dicarbonate (4.84 g, 22.20 mmol) were dissolved in dichloromethane (15 mL), and were added to the reaction. The resulting mixture was stirred for 30 min at room temperature, washed with 0.1 N HCl (25 mL) and the aqueous phase was extracted with dichloromethane (3×35 mL, monitored by TLC). The combined organic layers were dried with sodium sulfate, the solvents were removed under reduced pressure to obtain tert-butyl 6-fluoro-3-iodo-1H-indole-1-carboxylate (4.16 g, 11.52 mmol, 78% yield) as an orange solid. ¹H-NMR (CDCl₃) δ ppm: 7.82 (d, J=8.23 Hz, 1H), 7.68 (s 1H), 7.30-7.34 (m, 1H), 7.03-7.08 (m, 1H), 1.66 (s, 9H)

### Step 2.

Compound was prepared following the same procedure of (S)-tert-butyl 2-((((9H-fluoren-9-yl) methoxy) carbonyl)amino)-3-(isoquinolin-8-yl) propanoate. First Negishi coupling at 50° C. afforded the desired tert-butyl(S)-3-(2-((((9H-fluoren-9-yl) methoxy) carbonyl)amino)-3-(tert-butoxy)-3-oxopropyl)-7-fluoro-1H-indole-1-carboxylate (690 mg, 1.149 mmol, 57.4% yield) after purification by flash chromatography.

Analysis condition H: Retention time=3.885 min; ESI-MS(+) m/z [M-Boc-tBu+H]⁺: 445.2

### Step 3

Final product was obtained following the same procedure of (S)-2-((((9H-fluoren-9-yl) methoxy) carbonyl)amino)-3-(isoquinolin-8-yl) propanoic acid. TFA hydrolysis afforded the desired(S)-2-((((9H-fluoren-9-yl) methoxy) carbonyl)amino)-3-(7-fluoro-1H-indol-3-yl) propanoic acid as an off white powder (96 mg, 0.191 mmol, 16.63% yield) after purification by reverse phase prep HPLC (Column: 80 g size, Silisep C18, 19×150 mm, 5 μm, Mobile phases: A=10 mM ammonium acetate in water, B=MeoH. 15 mL/min flow Gradient: 0-20 min, 5-30% B, 20-55 min, 30-80% B, 55-60 min, 80-100% B, held at 100% B for 5 min. Compound was eluted at 75% B) followed by lyophilization.

Analysis condition F: Retention time=1.367 min; ESI-MS(+) m/z [M+H]⁺: 445.3. ¹H-NMR (400 MHz, DMSO-d6) δ ppm: 11.22 (s, 1H), 7.86 (d, J=8.72 Hz, 2H), 7.62-7.65 (m, 1H), 7.52-7.55 (m, 3H), 7.40-7.42 (m, 2H), 7.26-7.38 (m, 2H), 6.78-6.83 (m, 2H), 4.12-4.21 (m, 4H), 3.15-3.18 (m, 1H), 2.97-3.03 (m, 1H).

### Preparation of (2S,3S)-2-((((9H-fluoren-9-yl) methoxy) carbonyl)amino)-3-(1-(tert-butoxycarbonyl)-1H-indol-3-yl) butanoic acid

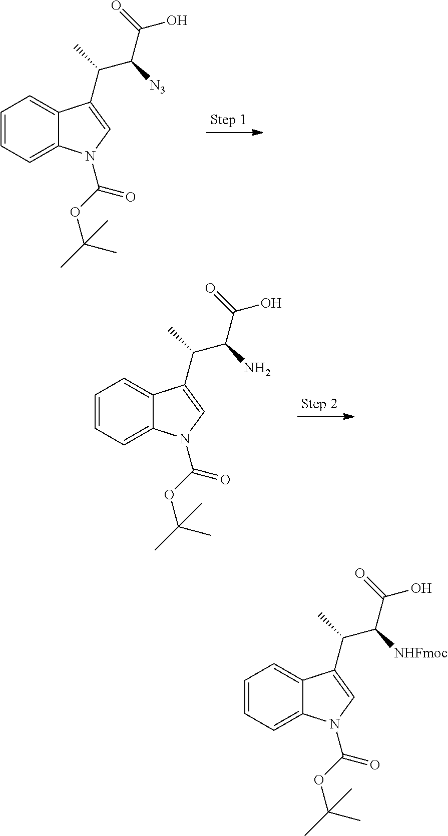

Compound (2S,3S)-2-azido-3-(1-(tert-butoxycarbonyl)-1H-indol-3-yl) butanoic acid was prepared following the procedure reported in Tetrahedron Letters 2001, 42, 4601-4603. The azide reduction step used different conditions as detailed below.

### Step 1.

To a solution of (2S,3S)-2-azido-3-(1-(tert-butoxycarbonyl)-1H-indol-3-yl) butanoic acid (1000 mg, 2.90 mmol) in THF (58 mL) was added platinum (IV) oxide (132 mg, 0.58 mmol). The reaction mixture was evacuated and filled with hydrogen. The reaction mixture was allowed to stir at room temperature with a hydrogen balloon for 2 h. The reaction mixture was evacuated and back filled with nitrogen three times. The solution was filtered through Celite®. The solvent was removed under vacuum and the crude residue was redissolved in EtOH. This solution was filtered through Celite® to give a clear solution which was concentrated under vacuum (0.89 g 96% yield). ¹H NMR (400 MHz, METHANOL-d₄) δ 8.13 (br d, J=8.0 Hz, 1H), 7.75 (d, J=7.8 Hz, 1H), 7.61 (s, 1H), 7.46-7.18 (m, 2H), 4.89 (s, 2H), 3.80 (d, J=6.5 Hz, 1H), 3.58 (t, J=7.2 Hz, 1H), 1.68 (s, 9H), 1.53 (d, J=7.3 Hz, 3H). Analysis condition B: Retention time=0.93 min; ESI-MS(+) m/z [M+H]⁺: 319.1.

### Step 2.

To a solution of (2S,3S)-2-amino-3-(1-(tert-butoxycarbonyl)-1H-indol-3-yl)butanoic acid (3.96 g, 12.44 mmol) in MeOH (25 mL) was added (9H-fluoren-9-yl)methyl 2,5-dioxopyrrolidine-1-carboxylate (888 mg, 2.76 mmol) followed by Et₃N (0.385 mL, 2.76 mmol). The reaction was stirred for 2 h at room temperature. The solvent was removed under vacuum and the residue was redissolved in EtOAc and washed with 1 N HCl aqueous solution then brine. The organic layer was collected, dried over anhydrous sodium sulfate, and concentrated under vacuum to give the desired product (1.3 g, 89% yield) which was not purified further. ¹H NMR (500 MHz, DMSO-d₆) δ 12.78 (br s, 1H), 8.07-7.80 (m, 2H), 7.76-7.48 (m, 4H), 7.46-7.15 (m, 6H), 5.75 (s, 1H), 4.44 (t, J=8.2 Hz, 1H), 4.33-4.22 (m, 1H), 4.19-4.07 (m, 2H), 1.56 (s, 9H), 1.39-1.27 (m, 3H). Analysis condition B: Retention time=1.27 min; ESI-MS(+) m/z [M+H]⁺: not observed.

### Preparation of (S)-2-((((9H-fluoren-9-yl) methoxy) carbonyl)amino)-3-(6-(o-tolyl)pyridin-3-yl) propanoic acid

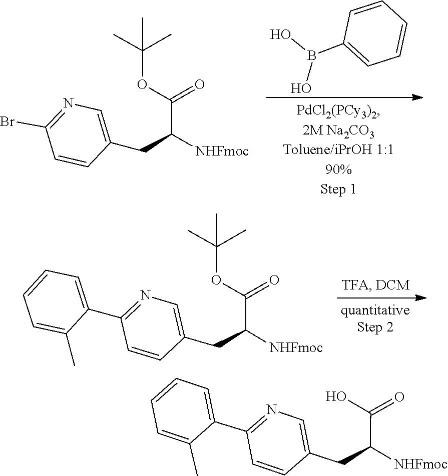

### Step 1.

To a stirred solution of tert-butyl(S)-2-((((9H-fluoren-9-yl) methoxy) carbonyl)amino)-3-(6-bromopyridin-3-yl) propanoate (1750 mg, 3.35 mmol) in toluene/iPrOH (1:1, v: v, 50 mL) was added o-tolylboronic acid (911.6 mg, 6.7 mmol) and 2M Na₂CO₃ aqueous solution (25.0 mL). The mixture was purged with argon three times. Dichlorobis(tricyclohexylphosphine) palladium (II) (123.6 mg, 0.167 mmol) was added and the reaction mixture was purged twice with argon. The reaction was heated to 80° C. for 20 h. The reaction was cooled to room temperature and iPrOH was removed by rotovap. The crude was partitioned between water and EtOAc. The aqueous phase was extracted with EtOAc. Organic phases were combined and dried over anhydrous MgSO₄. After filtration and concentration the crude product was obtained as a brown oil. Purification by flash chromatography using EtOAc: DCM (1:9) as eluant lead to tert-butyl(S)-2-((((9H-fluoren-9-yl) methoxy) carbonyl)amino)-3-(6-(o-tolyl)pyridin-3-yl) propanoate (1.81 g, 3.39 mmol, 90%) as a colorless oil.

### Step 2.

(S)-2-((((9H-Fluoren-9-yl) methoxy) carbonyl)amino)-3-(6-(o-tolyl)pyridin-3-yl) propanoate (1750 mg, 3.19 mmol) was dissolved in trifluoroacetic acid (5.00 mL) and the reaction was allowed to stir at room temperature for two hours. The reaction was brought to dryness on rotovap and the crude product was dissolved in diethyl ether and 1M HCl in diethyl ether. The mixture was sonicated for 2 hours to give a white solid. The product was isolated by filtration and washed with water to give(S)-2-((((9H-fluoren-9-yl) methoxy) carbonyl)amino)-3-(6-(o-tolyl)pyridin-3-yl) propanoic acid (1.91 g, 3.99 mmol, 100%) as a white solid. ¹H NMR (499 MHz, DMSO-d₆) δ 8.90 (s, 1H), 8.48 (br d, J=8.0 Hz, 1H), 7.96 (t, J=6.9 Hz, 2H), 7.89 (d, J=7.5 Hz, 2H), 7.64 (dd, J=7.2, 4.8 Hz, 2H), 7.52-7.45 (m, 1H), 7.43-7.29 (m, 7H), 4.46 (ddd, J=10.7, 8.9, 4.5 Hz, 1H), 4.25-4.15 (m, 3H), 3.45-3.34 (m, 1H), 3.18-3.10 (m, 1H), 3.08-3.00 (m, 1H), 2.27-2.20 (m, 3H).

### Preparation of (S)-2-((((9H-fluoren-9-yl) methoxy) carbonyl)amino)-3-(4′-acetamido-[1,1′-biphenyl]-4-yl) propanoic acid

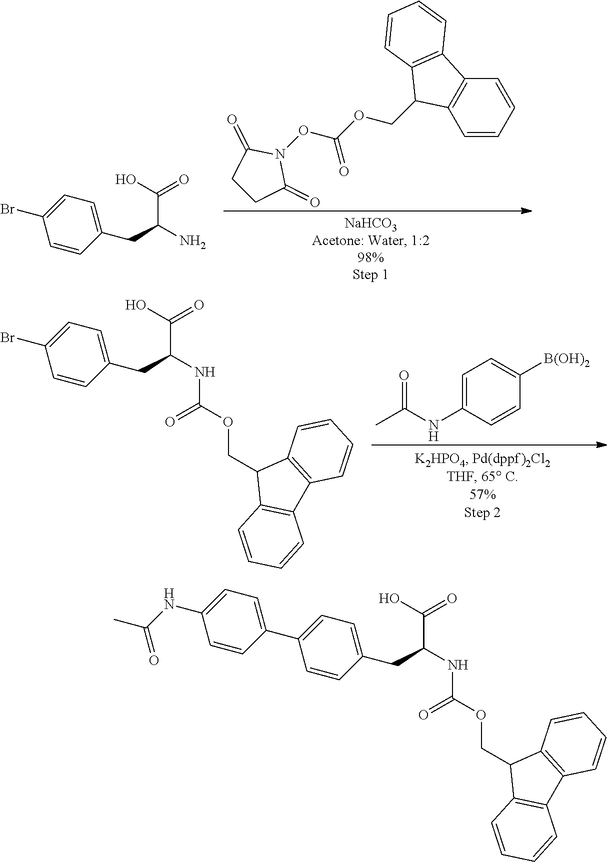

### Step 1.

A 5.0-l multi-neck round-bottomed flask was charged with(S)-2-amino-3-(4-bromophenyl) propanoic acid (150.0 g, 615 mmol), Fmoc-OSu (207 g, 615 mmol) in acetone (1500 mL), a solution of sodium bicarbonate (258 g, 3073 mmol) in water (3000 mL) in one lot and allowed to stir at room temperature for 16 h. The reaction mixture was slowly acidified with 10 N HCl solution to pH 1 and stirred for 15 min. The slurry was filtered and dried under vacuum and the cake was washed with water (3.0 L). Solids were dried for 16 h. The desired product was obtained as a white solid (280 g, 98%) and the product was taken to the next stage. Analysis condition E: Retention time=2.17 min; ESI-MS(+) m/z [M+H]⁺: 466.2.

### Step 2.

To a stirred solution of (S)-2-((((9H-fluoren-9-yl) methoxy) carbonyl)amino)-3-(4-bromophenyl) propanoic acid (1.0 g, 2.144 mmol) and (4-acetamidophenyl) boronic acid (0.576 g, 3.22 mmol) with THF (50 mL) in 150-ml pressure tube, Argon was purged for 5 min. Potassium phosphate, tribasic (1.366 g, 6.43 mmol) was then added and the purging was continued for another 5 min. 1,1′-bis(di-tert-butylphosphino) ferrocene palladium dichloride (0.140 g, 0.214 mmol) was then added, and the purging was continued for another 5 min. The reaction mixture was heated to 65° C. for 26 h. The reaction mass was diluted with EtOAc (25 mL) and washed with 10% citric acid aqueous solution (10 mL) and then brine solution to get the crude product. It was triturated with 20% DCM, stirred for 10 min and filtered with a buchner funnel, and then dried for 10 min. The crude was purified by flash chromatography to give 0.7 g (57%) of the desired product as a brown solid. Analysis condition E: Retention time=1.79 min; ESI-MS(+) m/z [M+H]⁺: 519.0. ¹H NMR (400 MHz, DMSO-d₆) δ 12.75 (br s, 1H), 9.99 (s, 1H), 7.87 (d, J=7.5 Hz, 2H), 7.77-7.49 (m, 9H), 7.47-7.22 (m, 7H), 4.26-4.13 (m, 4H), 3.11 (br dd, J=13.8, 4.3 Hz, 1H), 2.91 (dd, J=13.8, 10.8 Hz, 1H), 2.12-2.01 (m, 4H).

### Synthesis of Aryl/Heteroaryl Substituted Phenylalanines

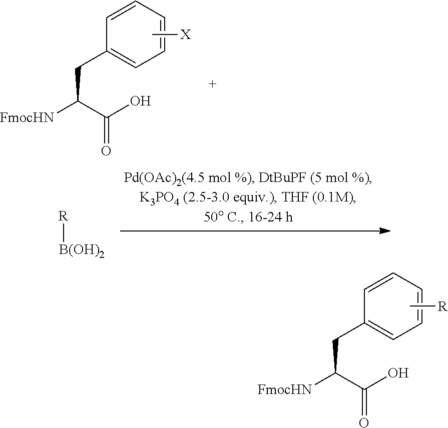

General procedures for Suzuki-Miyaura coupling (SMC) reactions in Scheme 1. To a N₂-flushed 20-mL scintillation vial equipped with a magnetic stir bar was added Fmoc-halo-Phe-OH (0.5 mmol), boronic acid (1.5-2.5 equiv.), and anhydrous THF (6 mL). The suspension was degassed by bubbling N₂ into the vial for several minutes. Palladium (II) acetate (4.5 mol %), DtBuPF (5 mol %), and then anhydrous K₃PO₄ (2.5 equiv.) were added. The suspension was degassed for several minutes, and then the vial was capped with a septum. The reaction mixture was stirred at 50° C. for 16 h. After cooling, 20% aqueous citric acid solution was added to acidify the reaction. The organic layer was separated, and the aqueous layer was extracted with EtOAc (2×). Silica gel was added to the combined organic layers, and the mixture was concentrated to dryness. The residue was dry-loaded on a silica gel column (ISCO system) and eluted with hexanes/EtOAc to give the desired product. Sometimes for compounds which are tailing in a Hexanes/EtOAc system, further eluting with MeOH/CH₂Cl₂ is also needed.

### Preparation of (S)-2-((((9H-fluoren-9-yl)methoxy)carbonyl)amino)-3-(4′-(tert-butoxycarbonyl)-[1,1′-biphenyl]-4-yl) propanoic acid

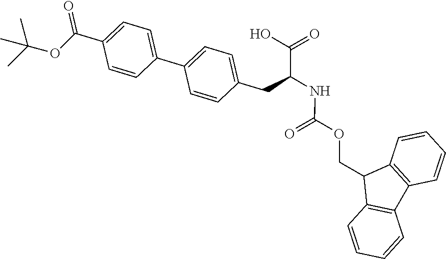 [SMILES: CC(C)(C)OC(=O)c1ccc(-c2ccc(C[C@H](NC(=O)OCC3c4ccccc4-c4ccccc43)C(=O)O)cc2)cc1]

(S)-2-((((9H-Fluoren-9-yl) methoxy) carbonyl)amino)-3-(4′-(tert-butoxycarbonyl)-[1,1′-biphenyl]-4-yl) propanoic acid was prepared according to the SMC general procedure. Yield: 78% (439 mg); colorless solids. ¹H NMR (400 MHz, methanol-d₄) δ 7.94 (d, J=8.3 Hz, 2H), 7.74 (d, J=7.6 Hz, 2H), 7.56 (d, J=8.4 Hz, 4H), 7.51 (d, J=8.1 Hz, 2H), 7.38-7.28 (m, 4H), 7.28-7.17 (m, 2H), 4.56-4.38 (m, 1H), 4.29 (dd, J=10.5, 7.0 Hz, 1H), 4.17 (dd, J=10.5, 7.1 Hz, 1H), 4.08 (t, J=7.0 Hz, 1H), 3.29-3.21 (m, 1H), 2.98 & 2.80 (dd, J=13.8, 9.6 Hz, total 1H), 1.59 (s, 9H). ESI-HRMS: Calcd for C₃₅H₃₄NO₆ [M+H]⁺ 564.23806, found 564.23896, mass difference 1.588 ppm.

### Preparation of (S)-2-((((9H-fluoren-9-yl) methoxy) carbonyl)amino)-3-(3′-(tert-butoxycarbonyl)-[1,1′-biphenyl]-4-yl) propanoic acid

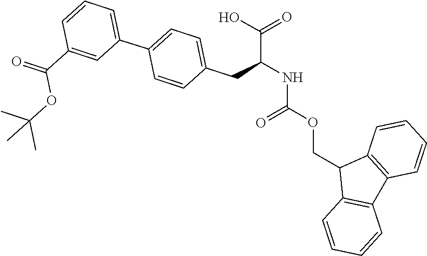 [SMILES: CC(C)(C)OC(=O)c1cccc(-c2ccc(C[C@H](NC(=O)OCC3c4ccccc4-c4ccccc43)C(=O)O)cc2)c1]

(S)-2-((((9H-Fluoren-9-yl) methoxy) carbonyl)amino)-3-(4′-(tert-butoxycarbonyl)-[1,1′-biphenyl]-4-yl) propanoic acid was prepared according to the SMC general procedure. Yield: 85% (240 mg); off-white solids. ¹H NMR (500 MHz, DMSO-d₆) δ 8.08 (t, J=1.8 Hz, 1H), 7.86 (dd, J=7.7, 1.4 Hz, 3H), 7.83 (d, J=8.1 Hz, 1H), 7.64 (d, J=7.7 Hz, 1H), 7.63 (d, J=7.5 Hz, 1H), 7.58-7.48 (m, 3H), 7.41-7.35 (m, 2H), 7.31 (d, J=7.8 Hz, 2H), 7.30-7.23 (m, 2H), 4.31-4.10 (m, 4H), 4.05 (td, J=8.2, 4.5 Hz, 1H), 3.13 & 2.9 (dd, J=13.6, 4.5 Hz, total 1H), 2.94 & 2.76 (dd, J=13.6, 8.7 Hz, total 1H), 1.56 (s, 9H). ESI-HRMS: Calcd for C₃₅H₃₇N₂O₆ [M+NH₄]⁺ 581.26461, found at 581.26474, mass difference 0.218 ppm.

### Preparation of (S)-2-((((9H-fluoren-9-yl) methoxy) carbonyl)amino)-3-(4-boronophenyl) propanoic acid

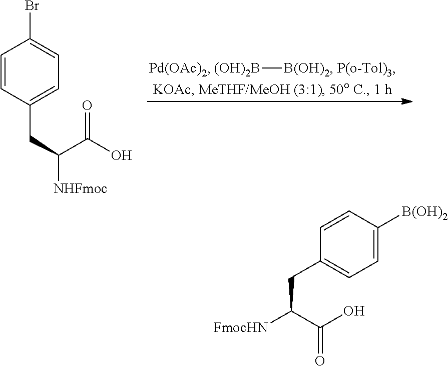

To a 75-ml pressure bottle(S)-2-((((9H-fluoren-9-yl) methoxy) carbonyl)amino)-3-(4-bromophenyl) propanoic acid (6.0 g, 12.87 mmol) and 2-methyl THF (250 mL) were charged, and the solution was purged with argon for 5 min. Tri-o-tolylphosphine (0.31 g, 1.03 mmol), tetrahydroxydiboron (2.31 g, 25.7 mmol), potassium acetate (3.79 g, 38.6 mmol) were added every in 10-min interval followed by the addition of MeOH (100 mL) and Pd(OAc)₂ (0.12 g, 0.52 mmol), and argon was purged for 10 min. The reaction was heated at 50° C. overnight. The reaction mixture was transferred into a 1-liter separatory funnel, diluted with 2-methyl-THF, and acidified with 1.5 N HCl to pH=2. The organic layer was washed with brine, dried (sodium sulphate), passed through celite, and concentrated to give black crude material. The crude was treated with petroleum ether to give a solid (10 g) which was dissolved with 2-methyl-THF and charcoal (2 g) was added. The mixture was heated on a rotovap without vacuum at 50° C. After filtration, the filtrate was passed through celite, concentrated. The resulting solid was treated with 30% ethyl acetate in petroleum ether, filtered to give 8 g of the crude as a fine off-white solid, which was further purified via flash chromatography then trituration with petroleum ether to give (S)-2-((((9H-fluoren-9-yl) methoxy) carbonyl)amino)-3-(4-boronophenyl) propanoic acid (4.0 g, 9.28 mmol, 72.1% yield) as a white solid. LCMS: 432.1 (M+H), tr=0.82 min. ¹H NMR (500 MHz, DMSO-d₆) δ 7.88 (d, J=7.6 Hz, 2H), 7.85-7.77 (m, 1H), 7.71 (br d, J=7.9 Hz, 3H), 7.68-7.60 (m, 2H), 7.41 (br d, J=6.6 Hz, 2H), 7.35-7.20 (m, 4H), 4.30-4.11 (m, 5H), 3.16-3.03 (m, 1H), 2.95-2.83 (m, 1H).

### Preparation of (S)-2-((((9H-fluoren-9-yl) methoxy) carbonyl)amino)-3-(4′-fluoro-[1,1′-biphenyl]-4-yl) propanoic acid

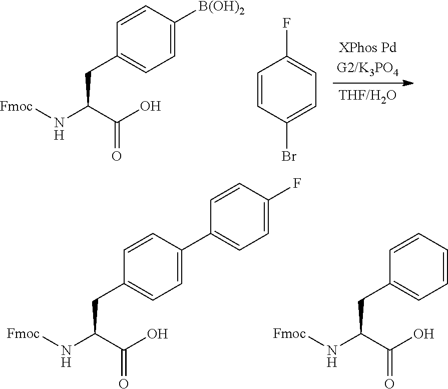

To a stirred solution of (S)-2-((((9H-fluoren-9-yl) methoxy) carbonyl)amino)-3-(4-boronophenyl) propanoic acid (217.5 mg, 0.504 mmol), 1-bromo-4-fluorobenzene (0.083 mL, 0.757 mmol) and XPhos Pd G2 (9.7 mg, 0.012 mmol) in THF (1 mL) at rt was added 0.5 M aqueous K₃PO₄ (2 mL, 1.000 mmol). N₂ was purged with vacuum three times and the mixture was stirred at 80° C. for 16 h. The mixture was cooled to rt. To the reaction was added 10% citric acid until pH<6. It was partitioned between EtOAc and H₂O, and the organic phase was separated, washed with brine, and dried over sodium sulfate. The mixture was filtered, SiO₂ (5 g) was added and concentrated. The material was then purified by flash chromatography (Teledyne ISCO CombiFlash R_{f}, gradient of 0% to 20% MeOH/CH₂Cl₂ over 15 column volumes, RediSep SiO₂ 40 g). Fractions containing the desired product were collected and concentrated to give(S)-2-((((9H-fluoren-9-yl) methoxy) carbonyl)amino)-3-(4′-fluoro-[1,1′-biphenyl]-4-yl) propanoic acid (206.1 mg, 0.43 mmol, 85% yield) as a cream solid: HPLC: RT=1.04 min (Waters Acquity UPLC BEH C18 1.7 μm 2.1×50 mm, CH₃CN/H₂O/0.05% TFA, 1 min. gradient, wavelength=254 nm); MS(ES): m/z=482 [M+H]⁺. ¹H NMR (499 MHz, DMSO-d₆) δ 12.78 (br s, 1H), 7.88 (d, J=7.5 Hz, 3H), 7.71-7.61 (m, 5H), 7.53 (d, J=8.1 Hz, 2H), 7.39 (q, J=7.3 Hz, 3H), 7.36-7.23 (m, 8H), 4.24-4.13 (m, 5H), 3.12 (dd, J=14.0, 4.5 Hz, 1H), 2.91 (dd, J=13.6, 10.3 Hz, 1H).

### Preparation of (S)-2-((((9H-fluoren-9-yl) methoxy) carbonyl)amino)-3-(3′,5′-difluoro-[1,1′-biphenyl]-4-yl) propanoic acid

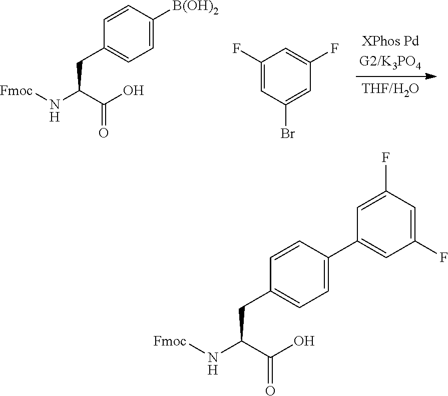

The final product was obtained following the same procedure of (S)-2-((((9H-fluoren-9-yl) methoxy) carbonyl)amino)-3-(4′-fluoro-[1,1′-biphenyl]-4-yl) propanoic acid. The Suzuki coupling reaction afforded the desired(S)-2-((((9H-fluoren-9-yl) methoxy) carbonyl)amino)-3-(3′,5′-difluoro-[1,1′-biphenyl]-4-yl) propanoic acid (197.1 mg, 0.40 mmol, 78% yield) as a colorless solid after purification by flash chromatography. HPLC: RT=1.06 min (Waters Acquity UPLC BEH C18 1.7 μm 2.1×50 mm, CH₃CN/H₂O/0.05% TFA, 1 min. gradient, wavelength-254 nm); MS(ES): m/z=500 [M+H]⁺. ¹H NMR (499 MHz, DMSO-d₆) δ 12.90-12.67 (m, 1H), 7.87 (d, J=7.5 Hz, 2H), 7.69-7.61 (m, 4H), 7.45-7.35 (m, 6H), 7.33-7.27 (m, 2H), 7.22-7.16 (m, 1H), 4.25-4.18 (m, 3H), 4.17-4.12 (m, 1H), 3.14 (dd, J=13.8, 4.4 Hz, 1H), 2.92 (dd, J=13.7, 10.6 Hz, 1H).

### Preparation of (S)-2-((((9H-fluoren-9-yl) methoxy) carbonyl)amino)-3-(3′,4′,5′-trifluoro-[1,1′-biphenyl]-4-yl) propanoic acid

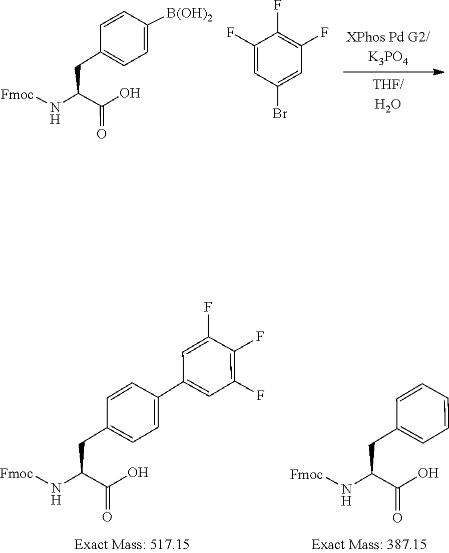

The final product was obtained following the same procedure of (S)-2-((((9H-fluoren-9-yl) methoxy) carbonyl)amino)-3-(4′-fluoro-[1,1′-biphenyl]-4-yl) propanoic acid. The Suzuki coupling reaction afforded the desired(S)-2-((((9H-fluoren-9-yl) methoxy) carbonyl)amino)-3-(3′,4′,5′-trifluoro-[1,1′-biphenyl]-4-yl) propanoic acid (218.5 mg, 0.422 mmol, 84% yield) as a colourless solid after purification by flash chromatography. HPLC: RT=1.466 min (Shimadzu UPLC with Waters Acquity BEH C18 1.7 μm 2.1×50 mm column, CH₃CN/H₂O/0.1% TFA, 3 min. gradient, wavelength=254 nm); MS(ES): m/z=556. ¹H NMR (499 MHz, DMSO-d₆) δ 12.79 (br s, 1H), 7.87 (d, J=7.6 Hz, 2H), 7.75 (d, J=8.6 Hz, 1H), 7.69-7.58 (m, 6H), 7.44-7.35 (m, 4H), 7.33-7.25 (m, 2H), 4.27-4.17 (m, 3H), 4.17-4.10 (m, 1H), 3.14 (dd, J=13.8, 4.4 Hz, 1H), 2.92 (dd, J=13.7, 10.7 Hz, 1H).

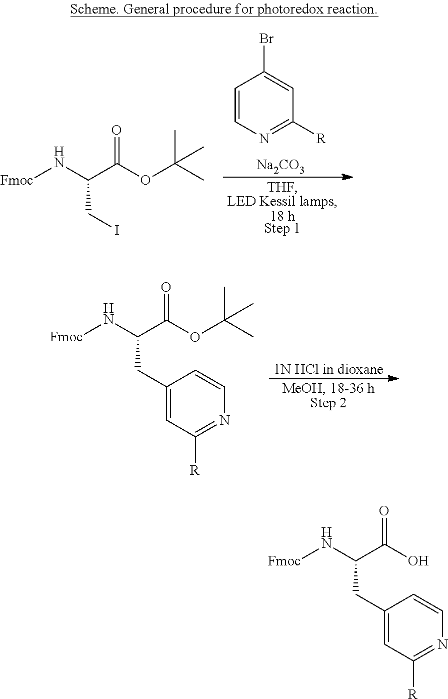

Ir[dF(CF₃)ppy₂]₂(dtbbpy)PF₆ (0.018 g, 0.016 mmol, 1 mol %), tert-butyl (R)-2-((((9H-fluoren-9-yl) methoxy) carbonyl)amino)-3-iodopropanoate (1.181 g, 2.393 mmol, 1.5 equiv), bromo-pyridine derivative (1.596 mmol, 1.00 equiv), pulverized Na₂CO₃ (0.338 g, 3.19 mmol, 2.00 equiv), and tris(trimethylsilane) silane (0.278 g, 1.596 mmol, 1.00 equiv) were charged into an oven-dried 40-mLl pressure-relief screw cap vial. The vial was capped, purged with nitrogen, diluted with THF (45.0 mL), and then sonicated. In a seperate vial were charged NiCl₂-glyme (18 mg, 0.080 mmol, 5 mol %) and di-tertbutylbipyridine (18 mg, 0.096 mmol, 6 mol %) in 1 mL dioxane. The vial was purged with nitrogen for 10 min. The Nickel-ligand complexe solution was transferred to the main reaction vial and the mixture was degassed with gentle nitrogen flow for 20 min. The reactor was sealed with parafilm and placed between 2 34 W blue LED Kessil lamps (ca. 7 cm away) and allowed to stir vigorously. After 16 h, the reaction was monitored by LCMS analysis. The resulting oil was dissolved into 4 M HCl dioxane solution (15 mL). After 16 h, the reaction mixture was brought to dryness on rotovap. The crude product was dissolved in a minimum amount of methanol and dry loaded on silica gel column for purification.

### Preparation of (2S)-2-({[(9H-fluoren-9-yl) methoxy]carbonyl}amino)-3-(2-methoxypyridin-4-yl) propanoic acid

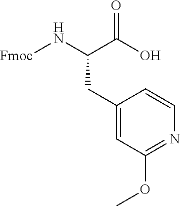

The mixture was rotovaped onto silica gel, purified by isco using 10% to 80% EtOAc/Hexanes. The fractions were pooled, concentrated to obtain the desired product as a clear oil (237 mg, 100%). Analysis conditions D: Retention time 1.74 min; ES+ 475.1.

### Preparation of ((S)-2-((((9H-fluoren-9-yl) methoxy) carbonyl)amino)-3-(4-(trifluoromethoxy)phenyl) propanoic acid

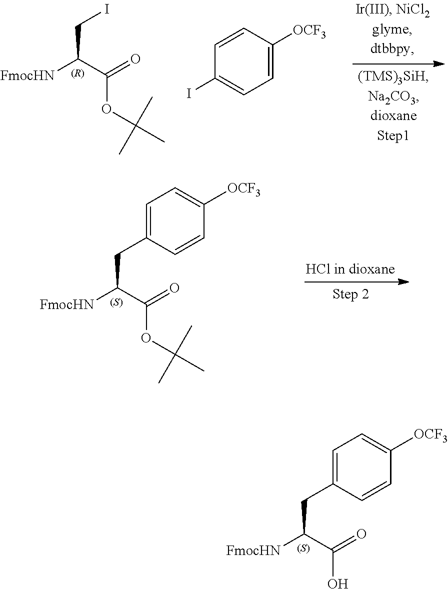

### Step 1.

In 4 separate 40-ml vials was placed Ir(dF(CF₃)ppy)₂(dtbbpy)PF₆ (5.6 mg, 4.99 μmol) and Na₂CO₃ (249 mg, 2.35 mmol) in dioxane (18 mL), and was fitted with a teflon screw cap and a stir bar. To the mixture was added 1-iodo-4-(trifluoromethoxy)benzene (0.16 mL, 1.02 mmol) stirred briefly, then tris(trimethylsilyl) silane (0.23 mL, 0.75 mmol) was added via syringe, and the suspension was degassed (cap on) with nitrogen for 5 min. To a separate 40-mL vial was added nickel (II) chloride ethylene glycol dimethyl ether complex (22 mg, 0.10 mmol) and 4,4′-di-tert-butyl-2,2′-bipyridine (33 mg, 0.12 mmol) ioxane (10 mL) was added and this solution was degassed (cap on) with nitrogen gas for 10 min and stirred. To the Ir mixture was added 2.5 mL of the Ni solution, and 5 mL of a solution of the iodo alanine, tert-butyl (R)-2-((((9H-fluoren-9-yl) methoxy) carbonyl)amino)-3-iodopropanoate (987 mg, 2.0 mmol) in dioxane (20 mL), and then the mixture was further degassed with nitrogen gas for another 5 min (cap on). The vials were sealed with parafilm, placed in the round photoredox reactor with light and fan on, stirred for 40 h. The eactions were removed from the illumination/reactor. The blackish reaction mixtures of each vial were poured into a 500-ml erlenmeyer flask into which was added EtOAc (200 mL). The mixture was filtered through celite, washed with EtOAc, and concentrated. The residue was purified by flash chromatography (Teledyne ISCO CombiFlash R_{f}, gradient of 0% using solvent A/B=CH₂C₁₂/EtOAcover 10 column volumes, RediSep SiO₂ 80 g loaded as DCM solution). The fractions containing the desired product were collected and concentrated to obtained the product tert-butyl(S)-2-((((9H-fluoren-9-yl) methoxy) carbonyl)amino)-3-(4-(trifluoromethoxy)phenyl) propanoate (865.2 mg, 1.64 mmol, 82% yield, only about 73% HPLC purity as a colourless oil and was used as was in the deprotection step: HPLC: RT=1.62 min (Waters Acquity UPLC BEH C18 1.7 μm 2.1×50 mm, CH₃CN/H₂O/0.05% TFA, 1 min. gradient, wavelength=254 nm); MS(ES): m/z=550 [M+23]+

### Step 2.

To a stirred solution of tert-butyl(S)-2-((((9H-fluoren-9-yl) methoxy) carbonyl)amino)-3-(4-(trifluoromethoxy)phenyl) propanoate (865.2 mg, 1.64 mmol) in dichloromethane (8.2 mL) at rt was added HCl (4M in dioxane, 8.20 mL, 32.8 mmol). The mixture was stirred at rt for 18 h. The mixture was concentrated in vacuo then dried under vacuum. The residue was dissolved in DMF (4 mL), purified on ISCO ACCQ Prep over 2 injections. The fractions containing the desire product were combined and partially concentrated on rotovap, then blown air over mixture over weekend. The residue was dissolved in CH₃CN, diluted with water, frozen, and lyophilized. To obtained the product(S)-2-((((9H-fluoren-9-yl) methoxy) carbonyl)amino)-3-(4-(trifluoromethoxy)phenyl) propanoic acid (344.1 mg, 0.73 mmol, 44.5% yield) as a colorless solid. HPLC: RT=1.38 min (Waters Acquity UPLC BEH C₁₈ 1.7 um 2.1×50 mm, CH₃CN/H₂O/0.05% TFA, 1.5 min. gradient, wavelength=254 nm); MS(ES): m/z=472 [M+1]+. ¹H NMR (499 MHz, DMSO-d₆) ppm δ 7.88 (d, J=7.5 Hz, 2H), 7.63 (d, J=7.4 Hz, 2H), 7.44-7.37 (m, 2H), 7.35-7.25 (m, 4H), 7.19 (br d, J=7.6 Hz, 3H), 4.30-4.20 (m, 1H), 4.21-4.13 (m, 2H), 4.04 (br d, J=3.5 Hz, 1H), 3.11 (br dd, J=13.6, 4.4 Hz, 1H), 2.91 (br dd, J=13.6, 9.1 Hz, 1H).

### Preparation of (S)-2-((((9H-fluoren-9-yl) methoxy) carbonyl)amino)-3-(2,5-dimethylphenyl) propanoic acid

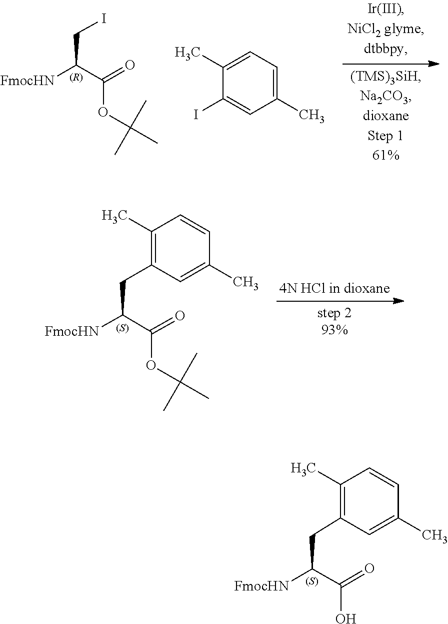

### Step 1.

Compound was prepared following the same procedure of tert-butyl(S)-2-((((9H-fluoren-9-yl) methoxy) carbonyl)amino)-3-(4-(trifluoromethoxy)phenyl) propanoate. The photoredox coupling afforded the desired product, tert-butyl(S)-2-((((9H-fluoren-9-yl) methoxy) carbonyl)amino)-3-(2,5-dimethylphenyl) propanoate (140.5 mg, 0.298 mmol, 61.1% yield) after purification by flash chromatography. HPLC: RT=1.21 min (Waters Acquity UPLC BEH C18 1.7 μm 2.1×50 mm, CH₃CN/H₂O/0.05% TFA, 1 min. gradient, wavelength=254 nm); Analysis condition F: Retention time=1.21 min; ESI-MS(+) m/z [M-tBu+H]⁺: 416. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.07 (d, J=7.7 Hz, 1H), 6.98 (d, J=7.7 Hz, 1H), 6.96 (s, 1H), 4.58-4.51 (m, 1H), 4.39 (dd, J=10.5, 7.3 Hz, 1H), 4.34 (dd, J=10.5, 7.2 Hz, 1H), 4.24-4.19 (m, 1H), 3.10-3.01 (m, 2H), 2.34 (s, 3H), 2.28 (s, 3H), 1.40 (s, 8H).

### Step 2.

Final product was obtained following the same procedure of (S)-2-((((9H-fluoren-9-yl) methoxy) carbonyl)amino)-3-(4-(trifluoromethoxy)phenyl) propanoic acid. Removal of tBu ester with HCl/dioxane afforded the desired(S)-2-((((9H-fluoren-9-yl) methoxy) carbonyl)amino)-3-(2,5-dimethylphenyl) propanoic acid (115.2 mg, 0.277 mmol, 93% yield) as a cream solid after purification by reverse phase flash chromatography. HPLC: RT=1.03 min (Waters Acquity UPLC BEH C18 1.7 μm 2.1×50 mm, CH₃CN/H₂O/0.05% TFA, 1 min. gradient, wavelength=254 nm); MS(ES): m/z=416 [M+H]⁺. ¹H NMR (499 MHz, CHLOROFORM-d) δ 7.88 (d, J=7.4 Hz, 2H), 7.79 (br d, J=8.6 Hz, 1H), 7.67 (d, J=7.4 Hz, 1H), 7.64 (d, J=7.5 Hz, 1H), 7.41 (td, J=7.3, 4.2 Hz, 3H), 7.35-7.29 (m, 2H), 7.29-7.25 (m, 1H), 7.02 (br d, J=8.9 Hz, 2H), 6.91 (br d, J=7.4 Hz, 1H), 4.21-4.10 (m, 5H), 3.07 (dd, J=14.1, 4.4 Hz, 1H), 2.80 (dd, J=14.1, 10.3 Hz, 1H), 2.24 (s, 3H), 2.18 (s, 3H)

### Preparation of (S)-2-((((9H-fluoren-9-yl) methoxy) carbonyl)amino)-3-(4-fluoro-3-methylphenyl) propanoic acid

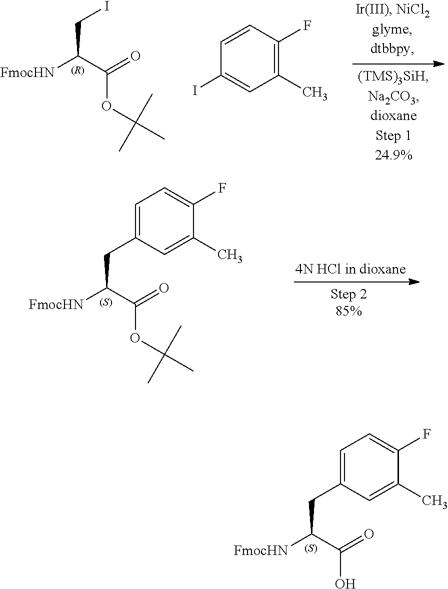

### Step 1.

The compound was prepared following the same procedure of tert-butyl(S)-2-((((9H-fluoren-9-yl) methoxy) carbonyl)amino)-3-(4-(trifluoromethoxy)phenyl) propanoate. The photoredox coupling afforded the desired product, tert-butyl(S)-2-((((9H-fluoren-9-yl) methoxy) carbonyl)amino)-3-(4-fluoro-3-(trifluoromethyl)phenyl) propanoate (66.3 mg, 0.13 mmol, 24.9% yield) as a colourless solid after purification by flash chromatography. HPLC: RT=1.19 min (Waters Acquity UPLC BEH C18 1.7 μm 2.1×50 mm, CH₃CN/H₂O/0.05% TFA, 1 min. gradient, wavelength=254 nm); MS(ES): m/z=474 [M-tBu]⁺. ¹H NMR (499 MHz, CHLOROFORM-d) δ 7.80 (d, J=7.5 Hz, 2H), 7.60 (dd, J=7.6, 3.3 Hz, 2H), 7.47-7.39 (m, 3H), 7.38-7.32 (m, 2H), 7.16-7.09 (m, 1H), 5.34 (br d, J=7.7 Hz, 1H), 4.57-4.47 (m, 2H), 4.40 (dd, J=10.3, 6.9 Hz, 1H), 4.26-4.21 (m, 1H), 3.14 (br d, J=4.9 Hz, 2H), 1.44 (s, 9H)

### Step 2.

Final product was obtained following the same procedure of (S)-2-((((9H-fluoren-9-yl) methoxy) carbonyl)amino)-3-(4-(trifluoromethoxy)phenyl) propanoic acid. Removal of the tBu ester with HCl/dioxane afforded the desired(S)-2-((((9H-fluoren-9-yl) methoxy) carbonyl)amino)-3-(4-fluoro-3-methylphenyl) propanoic acid (58.3 mg, 0.139 mmol, 85% yield) as a cream solid after purification by reverse phase flash chromatography. HPLC: RT=1.02 min (Waters Acquity UPLC BEH C18 1.7 μm 2.1×50 mm, CH₃CN/H₂O/0.05% TFA, 1 min. gradient, wavelength=254 nm); MS(ES): m/z=420 [M+H]⁺. ¹H NMR (499 MHz, DMSO-d₆) δ 12.86-12.66 (m, 1H), 7.89 (d, J=7.5 Hz, 2H), 7.73 (d, J=8.3 Hz, 1H), 7.65 (t, J=7.5 Hz, 2H), 7.42 (t, J=7.5 Hz, 2H), 7.35-7.26 (m, 2H), 7.17 (br d, J=7.5 Hz, 1H), 7.14-7.08 (m, 1H), 7.06-6.99 (m, 1H), 4.24-4.11 (m, 4H), 3.03 (dd, J=13.7, 4.3 Hz, 1H), 2.82 (dd, J=13.6, 10.6 Hz, 1H), 2.17 (s, 3H).

### Preparation of (S)-2-((((9H-fluoren-9-yl) methoxy) carbonyl)amino)-3-(2,4-difluoro-5-methoxyphenyl) propanoic acid

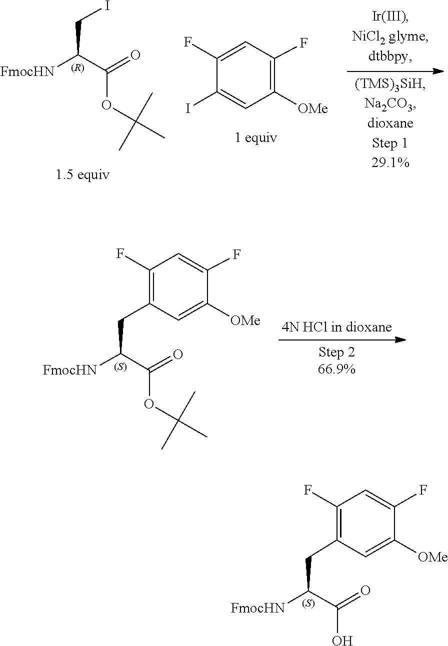

### Step 1.

The compound was prepared following the same procedure of tert-butyl(S)-2-((((9H-fluoren-9-yl) methoxy) carbonyl)amino)-3-(4-(trifluoromethoxy)phenyl) propanoate. The photoredox coupling afforded the desired product, tert-butyl(S)-2-((((9H-fluoren-9-yl) methoxy) carbonyl)amino)-3-(2,4-difluoro-5-methoxyphenyl) propanoate (77.1 mg, 0.151 mmol, 29.1% yield as a colourless solid after purification by flash chromatography. HPLC: RT=1.15 min (Waters Acquity UPLC BEH C18 1.7 μm 2.1×50 mm, CH₃CN/H₂O/0.05% TFA, 1 min. gradient, wavelength=254 nm); MS(ES): m/z=454 [M-t-Bu]+. ¹H NMR (499 MHZ, CHLOROFORM-d) δ 7.79 (d, J=7.4 Hz, 2H), 7.59 (t, J=6.4 Hz, 2H), 7.43 (t, J=7.3 Hz, 2H), 7.33 (td, J=7.5, 1.1 Hz, 3H), 6.85 (dd, J=10.8, 9.3 Hz, 1H), 6.83-6.79 (m, 1H), 5.40 (br d, J=8.1 Hz, 1H), 4.58-4.51 (m, 1H), 4.38 (dd, J=7.0, 4.5 Hz, 2H), 4.25-4.20 (m, 1H), 3.82 (s, 3H), 3.18-3.05 (m, 2H), 1.45 (s, 9H)

### Step 2.

The final product was obtained following the same procedure of (S)-2-((((9H-fluoren-9-yl) methoxy) carbonyl)amino)-3-(4-(trifluoromethoxy)phenyl) propanoic acid. Removal of tBu ester with HCl/dioxane afforded the desired(S)-2-((((9H-fluoren-9-yl) methoxy) carbonyl)amino)-3-(2,4-difluoro-5-methoxyphenyl) propanoic acid (45.9 mg, 0.101 mmol, 66.9% yield) as a cream solid after purification by reverse phase flash chromatography. HPLC: RT-0.99 min (Waters Acquity UPLC BEH C18 1.7 μm 2.1×50 mm, CH₃CN/H₂O/0.05% TFA, 1 min. gradient, wavelength=254 nm); MS(ES): m/z=454 [M+1]+. ¹H NMR (499 MHz, DMSO-d₆) δ 12.92 (br s, 1H), 7.89 (d, J=7.5 Hz, 2H), 7.71-7.65 (m, 1H), 7.63 (d, J=7.5 Hz, 2H), 7.41 (t, J=7.5 Hz, 2H), 7.34-7.25 (m, 2H), 7.24-7.15 (m, 2H), 4.24-4.12 (m, 4H), 3.77 (s, 3H), 3.16 (br dd, J=13.8, 4.6 Hz, 1H), 2.82 (dd, J=13.6, 10.7 Hz, 1H).

### Preparation of (S)-2-((((9H-fluoren-9-yl) methoxy) carbonyl)amino)-3-(2,3-dimethylphenyl) propanoic acid

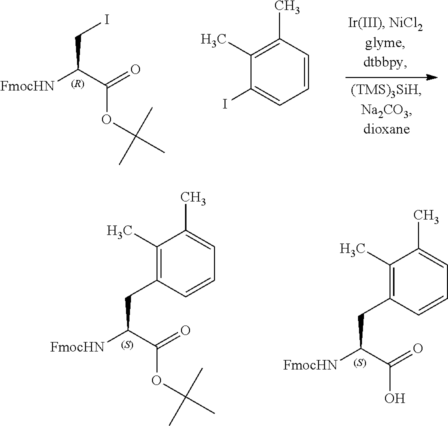

### Step 1.

The compound was prepared following the same procedure of tert-butyl(S)-2-((((9H-fluoren-9-yl) methoxy) carbonyl)amino)-3-(4-(trifluoromethoxy)phenyl) propanoate. The photoredox coupling afforded the desired product, tert-butyl(S)-2-((((9H-fluoren-9-yl) methoxy) carbonyl)amino)-3-(2,3-dimethylphenyl) propanoate (107.5 mg, 0.228 mmol, 55.5% yield) as a tan viscous oil after purification by flash chromatography. HPLC: RT=1.21 min (Waters Acquity UPLC BEH C18 1.7 μm 2.1×50 mm, CH₃CN/H₂O/0.05% TFA, 1 min. gradient, wavelength=254 nm); MS(ES): m/z=416 [M-t-Bu]+. ¹H NMR (499 MHz, CHLOROFORM-d) δ 7.79 (d, J=7.5 Hz, 2H), 7.61-7.56 (m, 2H), 7.42 (t, J=7.5 Hz, 2H), 7.35-7.31 (m, 2H), 7.09-7.06 (m, 1H), 7.02 (t, J=7.5 Hz, 1H), 7.00-6.96 (m, 1H), 5.30 (br d, J=8.3 Hz, 1H), 4.53 (q, J=7.4 Hz, 1H), 4.39 (dd, J=10.6, 7.3 Hz, 1H), 4.34 (dd, J=10.4, 7.0 Hz, 1H), 4.21 (t, J=7.2 Hz, 1H), 3.15 (dd, J=14.2, 7.0 Hz, 1H), 3.08 (dd, J=14.1, 7.3 Hz, 1H), 2.29 (s, 3H), 2.28 (s, 3H), 1.40 (s, 9H).

### Step 2.

The final product was obtained following the same procedure of (S)-2-((((9H-fluoren-9-yl) methoxy) carbonyl)amino)-3-(4-(trifluoromethoxy)phenyl) propanoic acid. Removal of the tBu ester with HCl/dioxane afforded the desired(S)-2-((((9H-fluoren-9-yl) methoxy) carbonyl)amino)-3-(2,3-dimethylphenyl) propanoic acid (72.9 mg, 0.175 mmol, 77% yield) as a cream solid after purification by reverse phase flash chromatography. HPLC: RT=1.03 min (Waters Acquity UPLC BEH C18 1.7 μm 2.1×50 mm, CH₃CN/H₂O/0.05% TFA, 1 min. gradient, wavelength=254 nm); MS(ES): m/z=416 [M+H]+. ¹H NMR (499 MHz, DMSO-d₆) δ 12.76 (br d, J=1.8 Hz, 1H), 7.89 (d, J=7.5 Hz, 2H), 7.79-7.71 (m, 1H), 7.66 (dd, J=13.6, 7.6 Hz, 2H), 7.42 (td, J=7.2, 4.1 Hz, 2H), 7.35-7.27 (m, 2H), 7.07 (d, J=7.3 Hz, 1H), 7.04-6.99 (m, 1H), 6.99-6.94 (m, 1H), 4.24-4.14 (m, 3H), 4.13-4.05 (m, 1H), 3.15 (dd, J=14.1, 4.1 Hz, 1H), 2.85 (dd, J=13.9, 10.4 Hz, 1H), 2.22 (s, 3H), 2.19 (s, 3H).

### Preparation of (S)-2-((((9H-fluoren-9-yl) methoxy) carbonyl)amino)-3-(2-fluoro-3-methylphenyl) propanoic acid

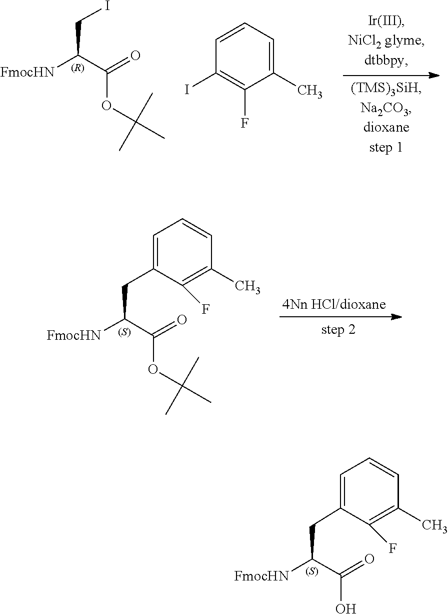

### Step 1

The compound was prepared following the same procedure of tert-butyl(S)-2-((((9H-fluoren-9-yl) methoxy) carbonyl)amino)-3-(4-(trifluoromethoxy)phenyl) propanoate. The photoredox coupling afforded the desired product, tert-butyl(S)-2-((((9H-fluoren-9-yl) methoxy) carbonyl)amino)-3-(2-fluoro-3-methylphenyl) propanoate (136.9 mg, LCMS showed 77% product and 23% impurity) as a viscous oil after purification by flash chromatography. Used as is, purify at after tBu hydrolysis.

### Step 2

The final product was obtained following the same procedure of (S)-2-((((9H-fluoren-9-yl) methoxy) carbonyl)amino)-3-(4-(trifluoromethoxy)phenyl) propanoic acid. Removal of tBu ester with HCl/dioxane afforded the desired(S)-2-((((9H-fluoren-9-yl) methoxy) carbonyl)amino)-3-(2-fluoro-3-methylphenyl) propanoic acid (79.7 mg, 0.190 mmol, 66.0% yield) as a cream solid after purification by reverse phase flash chromatography. HPLC: RT=1.02 min (Waters Acquity UPLC BEH C18 1.7 μm 2.1×50 mm, CH₃CN/H₂O/0.05% TFA, 1 min. gradient, wavelength=254 nm); MS(ES): m/z=420 [M+1]⁺. ¹H NMR (499 MHz, DMSO-d₆) δ 12.79 (br s, 1H), 7.89 (d, J=7.7 Hz, 2H), 7.78 (d, J=8.6 Hz, 1H), 7.65 (dd, J=11.6, 7.5 Hz, 2H), 7.44-7.39 (m, 3H), 7.37-7.25 (m, 3H), 7.14 (br t, J=7.4 Hz, 2H), 7.01-6.96 (m, 1H), 4.24-4.12 (m, 4H), 3.17 (dd, J=13.8, 4.8 Hz, 1H), 2.86 (dd, J=13.6, 10.8 Hz, 1H), 2.21 (s, 3H). 1H NMR and LCMS showed a 14% impurity.

### Preparation of ((S)-2-((((9H-fluoren-9-yl) methoxy) carbonyl)amino)-3-(2-fluoro-5-methylphenyl) propanoic acid

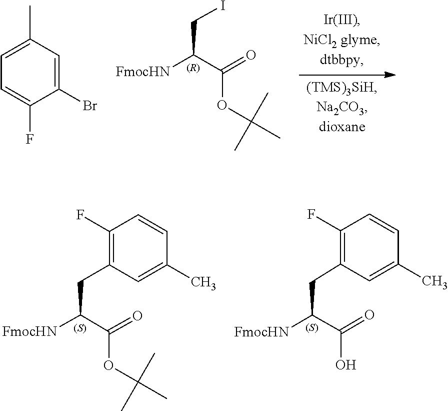

The compound was prepared following the same procedure of tert-butyl(S)-2-((((9H-fluoren-9-yl) methoxy) carbonyl)amino)-3-(4-(trifluoromethoxy)phenyl) propanoate. The photoredox coupling afforded the desired product, tert-butyl(S)-2-((((9H-fluoren-9-yl) methoxy) carbonyl)amino)-3-(2-fluoro-5-methylphenyl) propanoate (148.1 mg, 0.311 mmol, 65.4% yield) as a colourless gum after purification by flash chromatography. HPLC: RT=1.19 min (Waters Acquity UPLC BEH C18 1.7 μm 2.1×50 mm, CH₃CN/H₂O/0.05% TFA, 1 min. gradient, wavelength=254 nm); MS(ES): m/z=420 [M-t-Bu]+. ¹H NMR (499 MHz, CHLOROFORM-d) δ 7.79 (d, J=7.6 Hz, 2H), 7.60 (t, J=7.2 Hz, 2H), 7.42 (t, J=7.4 Hz, 2H), 7.37-7.30 (m, 2H), 7.06-6.99 (m, 2H), 6.97-6.90 (m, 1H), 5.41 (br d, J=8.1 Hz, 1H), 4.60-4.54 (m, 1H), 4.43 (dd, J=10.4, 7.2 Hz, 1H), 4.30 (dd, J=10.1, 7.5 Hz, 1H), 4.26-4.21 (m, 1H), 3.16 (dd, J=13.9, 6.7 Hz, 1H), 3.10 (dd, J=13.9, 6.4 Hz, 1H), 2.28 (s, 3H), 1.44 (s, 9H).

### Step 2

The final product was obtained following the same procedure of (S)-2-((((9H-fluoren-9-yl) methoxy) carbonyl)amino)-3-(4-(trifluoromethoxy)phenyl) propanoic acid. Removal of tBu ester with HCl/dioxane afforded the desired(S)-2-((((9H-fluoren-9-yl) methoxy) carbonyl)amino)-3-(2-fluoro-5-methylphenyl) propanoic acid (98.1 mg, 0.23 mmol, 75% yield) as a colourless solid after purification by reverse phase flash chromatography. HPLC: RT=1.01 min (Waters Acquity UPLC BEH C18 1.7 μm 2.1×50 mm, CH₃CN/H₂O/0.05% TFA, 1 min. gradient, wavelength=254 nm); MS(ES): m/z=420 [M+1]⁺. ¹H NMR (499 MHz, DMSO-d₆) δ 12.82 (br s, 1H), 7.89 (d, J=7.5 Hz, 2H), 7.78 (d, J=8.6 Hz, 1H), 7.67 (d, J=7.4 Hz, 1H), 7.64 (d, J=7.4 Hz, 1H), 7.42 (td, J=7.4, 3.0 Hz, 2H), 7.34-7.27 (m, 2H), 7.16-7.11 (m, 1H), 7.08-6.97 (m, 2H), 4.26-4.12 (m, 5H), 3.15 (dd, J=13.8, 4.9 Hz, 1H), 2.83 (dd, J=13.8, 10.3 Hz, 1H), 2.20 (s, 3H).

### Preparation of (S)-2-((((9H-fluoren-9-yl) methoxy) carbonyl)amino)-3-(2-fluoro-5-methoxyphenyl) propanoic acid

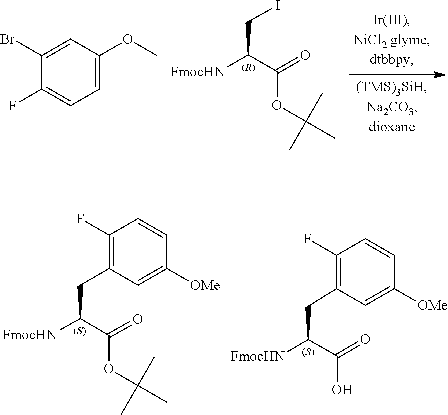

### Step 1

The compound was prepared following the same procedure of tert-butyl(S)-2-((((9H-fluoren-9-yl) methoxy) carbonyl)amino)-3-(2-fluoro-5-methoxyphenyl) propanoate (117.7 mg, 0.24 mmol, 50.4% yield) as a colourless solid after purification by flash chromatography. HPLC: RT=1.15 min (Waters Acquity UPLC BEH C18 1.7 μm 2.1×50 mm, CH₃CN/H₂O/0.05% TFA, 1 min. gradient, wavelength=254 nm); MS(ES): m/z=436 [M-t-Bu]⁺. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.01-6.93 (m, 1H), 6.79-6.72 (m, 2H), 5.41 (br d, J=8.2 Hz, 1H), 4.62-4.55 (m, 1H), 4.41 (dd, J=10.4, 7.3 Hz, 1H), 4.31 (dd, J=10.5, 7.4 Hz, 1H), 4.26-4.20 (m, 1H), 3.75 (s, 3H), 3.17 (dd, J=13.9, 6.7 Hz, 1H), 3.11 (dd, J=14.4, 6.6 Hz, 1H), 1.45 (s, 9H)

### Step 2

The final product was obtained following the same procedure of (S)-2-((((9H-fluoren-9-yl) methoxy) carbonyl)amino)-3-(4-(trifluoromethoxy)phenyl) propanoic acid. Removal of tBu ester with HCl/dioxane afforded the desired(S)-2-((((9H-fluoren-9-yl) methoxy) carbonyl)amino)-3-(2-fluoro-5-methoxyphenyl) propanoic acid (79.5 mg, 0.183 mmol, 76% yield) as a colourless solid after purification by flash chromatography. HPLC: RT=0.98 min (Waters Acquity UPLC BEH C18 1.7 μm 2.1×50 mm, CH₃CN/H₂O/0.05% TFA, 1 min. gradient, wavelength=254 nm); MS(ES): m/z=436 [M+1]+. Base peak of 214=fully deprotected amino acid fragment was also observed. ¹H NMR (499 MHz, DMSO-d₆) δ 12.84 (br s, 1H), 7.89 (d, J=7.5 Hz, 2H), 7.79 (d, J=8.6 Hz, 1H), 7.64 (t, J=8.4 Hz, 2H), 7.45-7.38 (m, 2H), 7.34-7.25 (m, 2H), 7.07 (t, J=9.2 Hz, 1H), 6.94 (dd, J=6.1, 3.2 Hz, 1H), 6.80 (dt, J=8.9, 3.6 Hz, 1H), 4.25-4.13 (m, 4H), 3.69 (s, 3H), 3.17 (dd, J=13.9, 4.6 Hz, 1H), 2.83 (dd, J=13.7, 10.7 Hz, 1H).

### Preparation of (S)-2-((((9H-fluoren-9-yl) methoxy) carbonyl)amino)-3-(2-methoxy-5-methylphenyl) propanoic acid

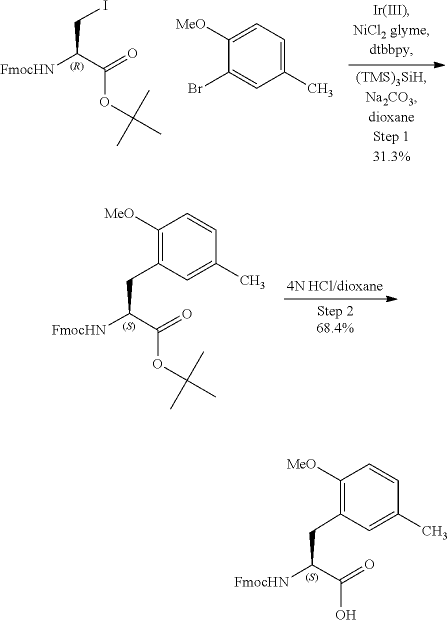

### Step 1.

The compound was prepared following the same procedure of tert-butyl(S)-2-((((9H-fluoren-9-yl) methoxy) carbonyl)amino)-3-(4-(trifluoromethoxy)phenyl) propanoate. The photoredox coupling afforded the desired product, tert-butyl(S)-2-((((9H-fluoren-9-yl) methoxy) carbonyl)amino)-3-(2-methoxy-5-methylphenyl) propanoate (73.9 mg, 0.15 mmol, 31.3% yield) as a colourless film after purification by flash chromatography. HPLC: RT=1.20 min (Waters Acquity UPLC BEH C18 1.7 μm 2.1×50 mm, CH₃CN/H₂O/0.05% TFA, 1 min. gradient, wavelength=254 nm); MS(ES): m/z=488 [M-tBu+H]⁺. ¹H NMR (499 MHZ, CHLOROFORM-d) δ 7.78 (d, J=7.6 Hz, 2H), 7.61-7.54 (m, 2H), 7.41 (t, J=7.4 Hz, 2H), 7.34-7.30 (m, 2H), 7.05 (dd, J=8.1, 1.5 Hz, 1H), 6.98 (d, J=1.4 Hz, 1H), 6.79 (d, J=8.3 Hz, 1H), 5.70 (br d, J=7.7 Hz, 1H), 4.49 (q, J=7.4 Hz, 1H), 4.33 (d, J=7.4 Hz, 2H), 4.25-4.18 (m, 1H), 3.82 (s, 3H), 3.10-3.02 (m, 2H), 2.26 (s, 3H), 1.43 (s, 9H) Step 2.

The final product was obtained following the same procedure of (S)-2-((((9H-fluoren-9-yl) methoxy) carbonyl)amino)-3-(4-(trifluoromethoxy)phenyl) propanoic acid. Removal of tBu ester with HCl/dioxane afforded the desired(S)-2-((((9H-fluoren-9-yl) methoxy) carbonyl)amino)-3-(2-methoxy-5-methylphenyl) propanoic acid (44.7 mg, 0.104 mmol, 68.4% yield) as a colourless solid after purification by flash chromatography. HPLC: RT=1.02 min (Waters Acquity UPLC BEH C18 1.7 μm 2.1×50 mm, CH₃CN/H₂O/0.05% TFA, 1 min. gradient, wavelength=254 nm); MS(ES): m/z=432 [M+H]⁺. ¹H NMR (499 MHz, DMSO-d₆) δ 12.61 (br s, 1H), 7.89 (d, J=7.5 Hz, 2H), 7.67 (d, J=7.5 Hz, 1H), 7.63 (d, J=7.5 Hz, 1H), 7.60 (br d, J=8.1 Hz, 1H), 7.42 (td, J=7.2, 3.5 Hz, 2H), 7.32 (td, J=7.5, 1.0 Hz, 1H), 7.30-7.26 (m, 1H), 7.02-6.97 (m, 2H), 6.84 (d, J=8.9 Hz, 1H), 4.26-4.10 (m, 4H), 3.75 (s, 3H), 3.12 (dd, J=13.5, 4.8 Hz, 1H), 2.72 (dd, J=13.4, 10.2 Hz, 1H), 2.16 (s, 3H).

### Preparation of (S)-2-((((9H-fluoren-9-yl) methoxy) carbonyl)amino)-3-hydroxy-3-methylbutanoic acid

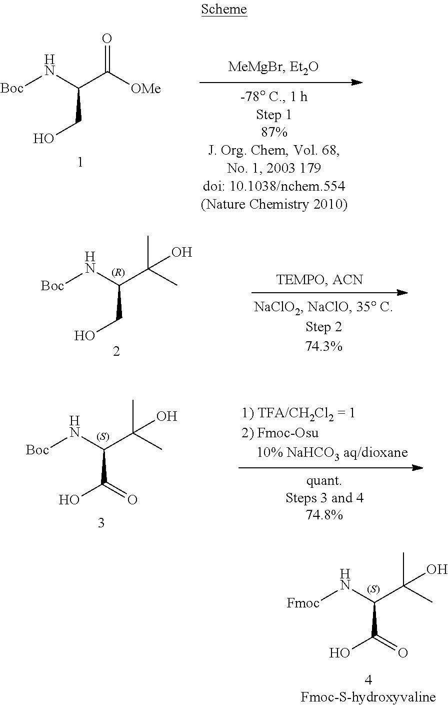

### Step 1.

To a 10-L multi-neck round-bottomed flask was charged methyl (tert-butoxycarbonyl)-D-serinate (50 g, 228 mmol), diethyl ether (4200 mL). The mixture was cooled to −78° C. and methylmagnesium bromide (456 mL, 1368 mmol) was added dropwise over 30 min. The reaction was stirred at RT for 1 h. It was cooled to 0° C. and saturated NH₄Cl solution (1500 mL), was added dropwise and stirred for 10 min. The organic layer was separated and the aqueous layer was extracted with ethyl acetate (3×2000 mL). The combined organic layer was washed with brine, dried over Na₂SO₄, and concentrated at 40° C. to give a colorless thick liquid. The crude was purified by I2PAC. Desired fractions were eluted at 50% EtOAc: petroleum ether mixture, and were collected and concentrated at 40° C. to give tert-butyl (R)-(1,3-dihydroxy-3-methylbutan-2-yl) carbamate (43.5 g, 87%) as a white solid. ¹H NMR (MeOD, 300 MHz) δ 3.70 (m, 1H), 3.48 (m, 1H), 3.21 (m, 1H), 1.35 (s, 9H), 1.13 (s, 3H), 1.05 (s, 3H).

### Step 2.

A 50-ml single neck round-bottomed flask was charged with tert-butyl (R)-(1,3-dihydroxy-3-methylbutan-2-yl) carbamate (43.0 g, 196 mmol), acetonitrile (650 mL) and was stirred till solution became clear. Sodium phosphate buffer (460 mL, 196 mmol) (pH=6.7, 0.67 M), (diacetoxyiodo)benzene (4.48 g, 13.92 mmol), and TEMPO (2.206 g, 14.12 mmol) were added sequentially and then the reaction was cooled to 0° C. and sodium chlorite (19.95 g, 221 mmol) was added. The color of the reaction turned black. The reaction was allowed to stir at 0° C. for 2 h. then at RT overnight. The orange colored reaction was quenched with saturated ammonium chloride solution (1000 mL) and the pH meter was used to adjust the pH=2 using 1.5 N HCl (330 mL). The aqueous solution was saturated with solid NaCl and extracted with ethyl acetate. The combined organic layer was washed with brine, dried over Na₂SO₄, and concentrated to obtain crude(S)-2-((tert-butoxycarbonyl)amino)-3-hydroxy-3-methylbutanoic acid (34.0 g, 74.3% yield) as an off-white solid and was taken directly to the next stage. ¹H NMR (MeOD, 300 MHz) δ 3.98 (s, 1H), 1.35 (s, 9H), 1.19 (s, 3H), 1.16 (9s, 3H).

### Step 3.

A 2000-mL single neck flask was charged with(S)-2-((tert-butoxycarbonyl)amino)-3-hydroxy-3-methylbutanoic acid (90 g, 386 mmol) dioxane (450 mL) and was cooled to 0° C. 4N HCl in Dioxane (450 mL, 1800 mmol) was added dropwise over 10 min. The reaction was allowed to stir at RT for 3 h. It was concentrated and azetroped with toluene (2×) then stirred with ethyl acetate for 10 min. It was filtered and dried under vacuum to obtain crude(S)-2-amino-3-hydroxy-3-methylbutanoic acid, HCl (70 g, 107% yield) as a white solid and was taken directly to the next step.

### Step 4

To a 3000-ml multi-neck round-bottomed flask was charged(S)-2-amino-3-hydroxy-3-methylbutanoic acid, HCl (70 g, 413 mmol), dioxane (1160 mL) and water (540 mL) The stirred solution became clear and a solution of sodium bicarbonate (104 g, 1238 mmol) in water (1160 mL) was added in one portion at RT. The reaction mass was allowed to stir at RT for 30 min. A solution of Fmoc-OSu (139 g, 413 mmol) in 1,4-dioxane (1460 mL) was added in one portion at RT. The reaction was allowed to stir at RT for 16 h. The reaction was concentrated to remove dioxane. To the resulting solution water was added and washed with ethyl acetate (3×1000 mL). The aqueous solution was acidified to pH 1-2 and extracted with ethyl acetate. The combined organic layer was washed with water, followed by brine, finally dried over Na₂SO₄, and concentrated to give an off-white solid (135.7 g). To remove the trapped dioxane and ethyl acetate the following procedure was followed: the solid was dissolved in ethyl acetate (1200 mL) and was stripped off with n-hexane (3000 mL). The slurry obtained was stirred for 10 min, filtered, dried under vacuum to give(S)-2-((((9H-fluoren-9-yl) methoxy) carbonyl)amino)-3-hydroxy-3-methylbutanoic acid (112.0 g, 74.8 yield for two steps) as a white solid.

### Preparation of (S)-2-((((9H-fluoren-9-yl) methoxy) carbonyl)amino)-3-(3,4,5-trifluorophenyl) propanoic acid

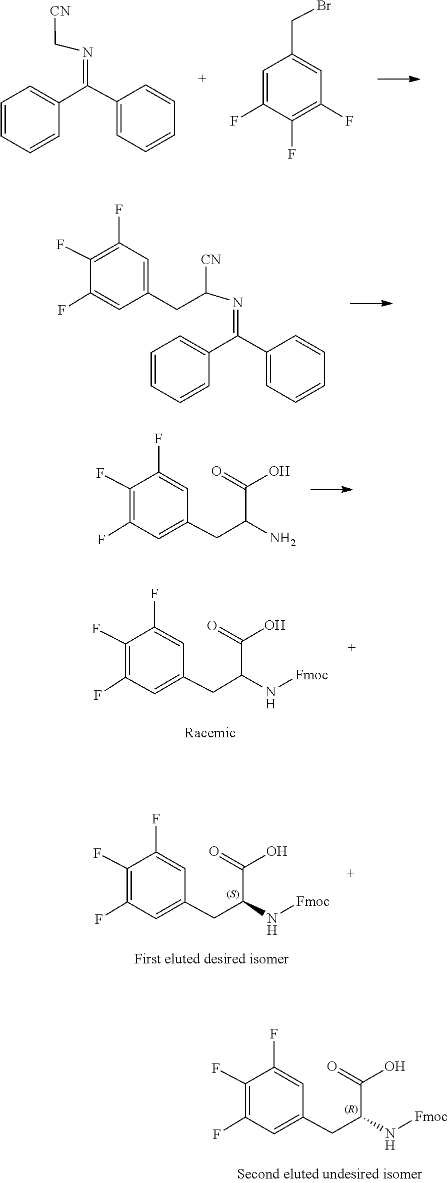

### Step 1.

To a stirred solution of 2-((diphenylmethylene)amino) acetonitrile (100 g, 454 mmol) in DCM (1000 mL), 5-(bromomethyl)-1,2,3-trifluorobenzene (66.5 mL, 499 mmol) and benzyltrimethylammonium chloride (16.86 g, 91 mmol) was added. To this, 10 M NaOH (136 mL, 1362 mmol) solution was added and stirred at rt overnight. After 26 h, the reaction mixture was diluted with water (500 mL) and the DCM layer was separated. The aqeuous layer was further extracted with DCM (2×250 mL). The organic layer was combined, washed with water and brine solution, dried over Na₂SO₄, filtered, and concentrated under vacuum. The crude compound was purified by flash column chromatography (1.5 kg, silica gel, 0-10% ethylacetate/petroleum ether mixture) and the desired fractions were collected and concentrated to afford 2-((diphenylmethylene)amino)-3-(3,4,5-trifluorophenyl) propanenitrile (140 g, 384 mmol, 85% yield) as a yellow solid. Analysis condition E: Retention time=3.78 min; ESI-MS(+) m/z [M+H]⁺: 365.2.

### Step 2.

To a stirred solution of 2-((diphenylmethylene)amino)-3-(3,4,5-trifluorophenyl) propanenitrile (80 g, 220 mmol) in 1,4-dioxane (240 mL), was added conc. HCl (270 mL, 3293 mmol) and the mixture was stirred at 90° C. for 16 h. The reaction mixture was taken as such for next step.

### Step 3.

To the crude aqueous dioxane solution from the previous was added 10 N NaOH solution until the solution was neutral. Na₂CO₃ (438 ml, 438 mmol) was then added, followed by the addition of Fmoc-OSu (81 g, 241 mmol). The mixture was stirred at rt overnight. The aqueous solution was acidified with 1.5 N HCl till pH=2 and the solid formed was filtered, dried to afford the crude compound. It was slurried initially with 5% EtOAc/petroleum ether for 30 min and filtered. The filtered compound was further slurried with ethyl acetate for 20 min and filtered to get the crude racemic 2-((((9H-fluoren-9-yl) methoxy) carbonyl)amino)-3-(3,4,5-trifluorophenyl) propanoic acid (90 g, 204 mmol, 93% yield) as an off-white solid. This racemic compound was separated into two isomers by SFC purification to get the desired isomers. (conditions) After concentration of the desired isomer, it was slurried with 5% EtOAc/petroleum ether and filtered to get(S)-2-((((9H-fluoren-9-yl) methoxy) carbonyl)amino)-3-(3,4,5-trifluorophenyl) propanoic acid (43 g, 95 mmol, 43.3% yield) as an off-white solid. ¹H NMR (MeOD, 400 MHz) δ 7.78 (d, J=7.2 Hz, 2H), 7.60 (t, J=8.0 Hz, 2H), 7.38 (t, J=8.0 Hz, 2H), 7.28 (t, J=7.6 Hz, 2H), 7.01 (t, J=7.8 Hz, 2H), 4.48-4.26 (m, 3H), 4.18 (m, 1H), 3.18 (m, 1H), 2.91 (m, 1H). ¹⁹F (MeOD, 376 MHz) δ −137.56 (d, J=19.6 Hz, 2F), −166.67 (t, J=19.6 Hz, 1F). Analysis condition E: Retention time=3.15 min; ESI-MS(+) m/z [M+H]⁺: 442.2.

The other fraction was concentrated to get (R)-2-((((9H-fluoren-9-yl) methoxy) carbonyl)amino)-3-(3,4,5-trifluorophenyl) propanoic acid (40 g, 91 mmol, 41.4% yield) as an off-white solid.

### Preparation of (S)-2-((((9H-fluoren-9-yl) methoxy) carbonyl)amino)-4-(tert-butoxy)-3,3-dimethyl-4-oxobutanoic acid

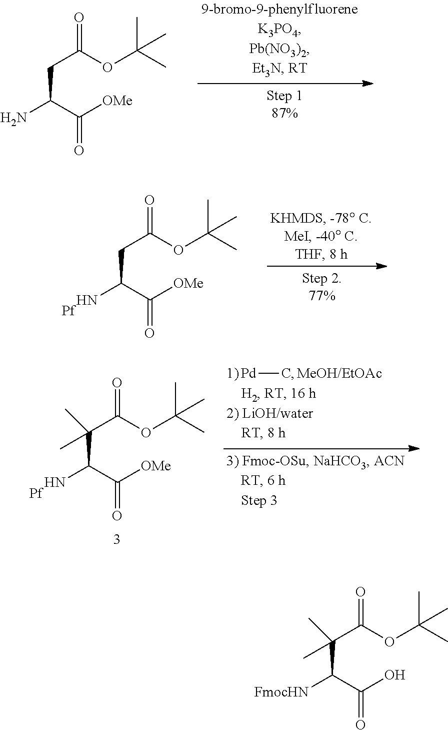

### Step 1.

To a stirred solution of 4-(tert-butyl) 1-methyl L-aspartate, HCl salt (34 g, 142 mmol) in acetonitrile (550 mL), was added lead (II) nitrate (47.0 g, 142 mmol), potassium phosphate (66.2 g, 312 mmol), and TEA (19.77 mL, 142 mmol) under nitrogen atmosphere. The mixture was cooled to 0° C. then a solution of 9-bromo-9-phenylfluorene (43.3 g, 135 mmol) in acetonitrile (100 mL) was added. The reaction mixture was stirred at RT for 48 h and the reaction progress was monitored by TLC (50% EA in PE) and LCMS. The reaction mixture was filtered over celite, washed with chloroform, and evaporated to get thick pale yellow liquid, to which ethyl acetate (3500 mL) was added. The EtOAc layer was washed with 5% citric acid solution (500 mL) followed by brine solution. The organic layer was dried over sodium sulfate and evaporated under reduced pressure to get pale yellow thick liquid, which was scratched with petroleum ether and filtered to obtain 4-(tert-butyl) 1-methyl (9-phenyl-9H-fluoren-9-yl)-L-aspartate (55 g, 124 mmol, 87% yield) as a white solid. Analysis condition L: Retention time=1.73 min; ESI-MS(+) m/z [M+Na]⁺: 466.40.

### Step 2.

A solution of 4-(tert-butyl) 1-methyl (9-phenyl-9H-fluoren-9-yl)-L-aspartate (22.5 g, 50.7 mmol) was cooled to −78° C. under Ar and a solution of KHMDS (127 mL, 127 mmol, 1 M in THF) was added over 30 min while stirring. The reaction was allowed to warm to −40° C., and methyl iodide (9.52 mL, 152 mmol) was added dropwise. The reaction was stirred at −40° C. for 5 h. The reaction was monitored by TLC and LCMS. Saturated NH₄Cl (400 mL) was added followed by H₂O (100 mL). The resulting mixture was extracted with EtOAc (3×) and the combined organic extracts were washed with 2% citric acid (200 mL), aq. NaHCO₃ (200 mL), and brine. The organic layer was dried over anhydrous Na₂SO₄, evaporated in vacuo, and recrystallized from hexanes to give 1-(tert-butyl) 4-methyl(S)-2,2-dimethyl-3-((9-phenyl-9H-fluoren-9-yl)amino) succinate (18.5 g, 39.2 mmol, 77% yield) as a white solid, which was taken for next step. Analysis condition L: Retention time=2.04 min; ESI-MS(+) m/z [M+Na]⁺: 494.34.

### Step 3.

A stirred solution of 1-(tert-butyl) 4-methyl(S)-2,2-dimethyl-3-((9-phenyl-9H-fluoren-9-yl)amino) succinate (24 g, 50.9 mmol) in methanol (270 mL) and ethyl acetate (100 mL) was degassed with nitrogen. Pd—C (2.71 g, 2.54 mmol) (10% by weight) was added, and the mixture was flushed with hydrogen gas and then stirred at RT in 1-liter capacity autoclave with 50 psi overnight. The reaction mixture was filtered through celite pad, washed with a mixture of methanol and ethyl acetate. The combined solvents were evaporated to dryness and the precipitated white solid was removed by filtration to obtain a pale yellow liquid 1-(tert-butyl) 4-methyl (S)-3-amino-2,2-dimethylsuccinate (11.7 g) which was taken as such for the next step.

### Step 4.

To a stirred solution of 1-(tert-butyl) 4-methyl(S)-3-amino-2,2-dimethylsuccinate (11.0 g, 47.6 mmol) cooled in an ice bath, was added lithium hydroxide (428 mL, 86 mmol, 0.2 M solution in water) and the reaction was slowly brought to RT. The reaction was monitored by TLC and LCMS. The reaction mixture was evaporated and directly taken to the next step. To a stirred solution of (S)-2-amino-4-(tert-butoxy)-3,3-dimethyl-4-oxobutanoic acid (15 g, 69.0 mmol) (which was in water from the previous batch) in acetonitrile (200 mL) cooled to 0° C., was added sodium bicarbonate (5.80 g, 69.0 mmol) and Fmoc-OSu (46.6 g, 138 mmol). The reaction mixture was stirred at RT overnight. It was acidified with 2 N HCl to pH=4, then extracted with ethyl acetate (3×500 mL), and the combined organic layer was washed with brine, dried over sodium sulfate, and evaporated to get an off-white solid, which was purified by ISCO flash chromatography with 20% EA in petroleum ether to get(S)-2-((((9H-fluoren-9-yl) methoxy) carbonyl)amino)-4-(tert-butoxy)-3,3-dimethyl-4-oxobutanoic acid (12.2 g, 26.9 mmol, 39.0% yield) as a white solid. 1HNMR (CDCl₃, 400 MHz) δ 7.77 (d, J=7.6 Hz, 2H), 7.60 (m, 2H), 7.42 (t, J=8.0 Hz, 2H), 7.33 (t, J=7.6 Hz, 2H), 4.65 (m, 2H), 4.34 (m, 1H), 4.25 (m, 1H), 3.18 (m, 1H), 1.40-1.27 (m, 6H). Analysis condition E: Retention time=1.90 min; ESI-MS(+) m/z [M+H]⁺: 440.2.

### Preparation of (S)-2-((((9H-fluoren-9-yl) methoxy) carbonyl)amino)-3-(3-(tert-butoxycarbonyl)phenyl) propanoic acid

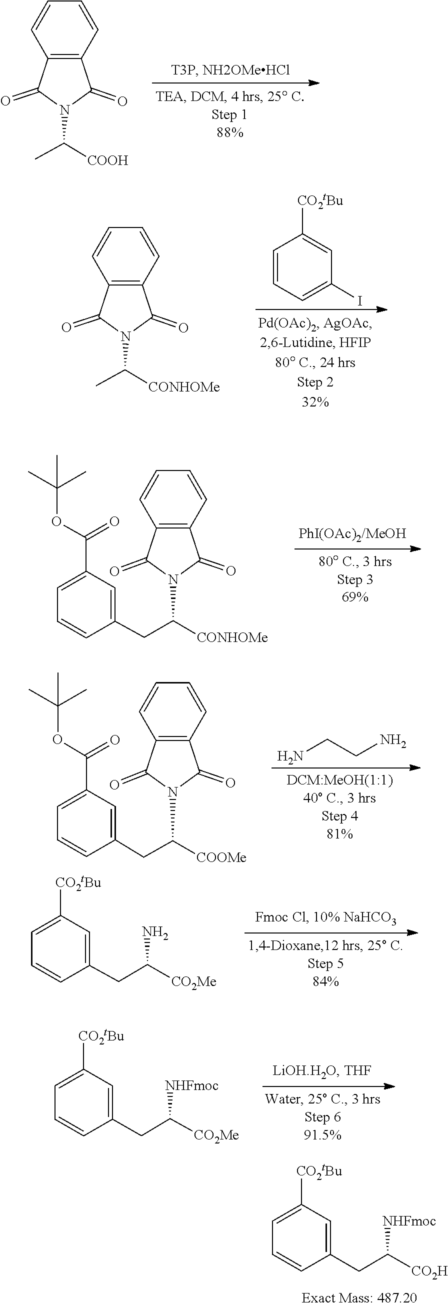

### Step 1.

To a solution of (S)-2-(1,3-dioxoisoindolin-2-yl) propanoic acid (80 g, 365 mmol), O-methylhydroxylamine hydrochloride (36.6 g, 438 mmol) in CH₂Cl₂ (2000 mL), was added TEA (153 mL, 1095 mmol) at RT. The reaction was cooled to 0° C., 1-propanephosphonic anhydride (326 mL, 547 mmol) was added dropwise. The reaction was stirred at RT for 2 h. It was quenched with saturated ammonium chloride (500 mL) and extracted with EtOAc (3×300 mL). The combined organic layers were washed with saturated brine, dried over Na₂SO₄, and concentrated under reduced pressure. The crude product was purified via combiflash using 120 g silica column with 38 to 45% EtOAc in petroleum ether to give(S)-2-(1,3-dioxoisoindolin-2-yl)-N-methoxypropanamide (80 g, 322 mmol, 88% yield). ¹H NMR (DMSO-d₆, 400 MHz) δ 11.36 (s, 1H), 7.91-7.85 (m, 4H), 4.75-4.69 (m, 1H), 3.56 (s, 3H), 1.51 (d, J=7.6 Hz, 3H). (A082E-536-01)

### Step 2.

To a solution of (S)-2-(1,3-dioxoisoindolin-2-yl)-N-methoxypropanamide (20 g, 81 mmol), palladium (II) acetate (1.809 g, 8.06 mmol), silver acetate (26.9 g, 161 mmol) placed in a 1000-ml seal tube, was added tert-butyl 3-iodobenzoate (36.8 g, 121 mmol), 2,6-Lutidine (2.395 ml, 24.17 mmol), HFIP (300 ml) at 25° C. under N₂ atmosphere. The reaction was stirred for 15 min at 25° C. under N₂ and then heated Up to 80° C. for 24 h with vigorous stirring. The reaction mixture was filtered through celite and washed with DCM (200 mL). The combined organic layer was concentrated under reduced pressure. The crude product was purified via combiflash using 220 g silica column eluting with 25 to 30% EtOAc: CHCl₃ to obtain the desired product tert-butyl(S)-3-(2-(1,3-dioxoisoindolin-2-yl)-3-(methoxyamino)-3-oxopropyl)benzoate (11 g, 25.9 mmol, 32.2% yield). Analysis condition E: Retention time=2.52 min; ESI-MS(+) m/z [M−H]⁺: 423.2. ¹H NMR (DMSO-d₆, 400 MHz) δ 11.46 (s, 1H), 7.82 (m, 4H), 7.63 (d, J=7.6 Hz, 1H), 7.54 (s, 1H), 7.40 (d, J=7.6 Hz, 1H), 7.30 (t, J=7.6 Hz, 1H), 4.93-4.89 (m, 1H), 3.59 (s, 3H), 3.56-3.49 (m, 1H), 3.36-3.27 (m, 1H), 1.40 (s, 9H),

### Step 3.

To a solution of tert-butyl(S)-3-(2-(1,3-dioxoisoindolin-2-yl)-3-(methoxyamino)-3-oxopropyl)benzoate (15 g, 35.3 mmol) in methanol (200 mL), (diacetoxyiodo)benzene (12.52 g, 38.9 mmol) was added at RT. The temperature was slowly raised to 80° C. and stirred for 3 h at 80° C. The Reaction was concentrated under reduced pressure to get the crude product. It was purified with silica gel chromatography (100-200 mesh eluting with 20% EA: hexane) to obtain the desired compound tert-butyl(S)-3-(2-(1,3-dioxoisoindolin-2-yl)-3-methoxy-3-oxopropyl)benzoate (10 g, 24.42 mmol, 69.1% yield. ¹H NMR (CDCl₃, 400 MHz) δ 7.80-7.76 (m, 4H), 7.72-7.68 (m, 2H), 7.34-7.26 (m, 1H), 7.25-7.23 (m, 1H), 5.14 (dd, J=10.8, 5.6 Hz, 1H), 3.76 (s, 3H), 3.65-3.49 (m, 2H), 1.50 (s, 9H). (A082E-552-01)

### Step 4.

To a solution of tert-butyl(S)-3-(2-(1,3-dioxoisoindolin-2-yl)-3-methoxy-3-oxopropyl)benzoate (15 g, 36.6 mmol) in methanol (25 mL) ethylenediamine (12.25 mL, 183 mmol) was added at RT. The reaction temperature was slowly raised to 40° C. and stirred for 3 h at 40° C. The mixture was concentrated under reduced pressure to get the crude product. It was purified with silica gel chromatography (100-200 mesh eluting with 20% EA: hexane) to obtain the desired compound tert-butyl(S)-3-(2-amino-3-methoxy-3-oxopropyl)benzoate (8.3 g, 29.7 mmol, 81% yield). ¹H NMR (DMSO-d₆, 400 MHz) δ 8.32 (s, 1H), 7.77-7.72 (m, 2H), 7.46-7.38 (m, 1H), 3.61-3.57 (m, 4H), 2.96-2.91 (m, 1H), 2.85-2.82 (m, 1H), 1.79 (br. s, 2H), 1.55 (s, 9H). (A082E-555-01)

### Step 5.

To a solution of tert-butyl(S)-3-(2-amino-3-methoxy-3-oxopropyl)benzoate (10 g, 35.8 mmol) in dioxane (150 mL), sodium bicarbonate (6.01 g, 71.6 mmol) was added followed by the addition of 9-fluorenylmethyl chloroformate (13.89 g, 53.7 mmol) at RT. The reaction was stirred for 12 h at RT. It was diluted with water and extracted with ethyl acetyate. The organic layer was concentrated under reduced pressure to get the crude product. It was purified via silica gel chromatography (100-200 mesh eluting with 20% EA: hexane) to obtain the desired compound tert-butyl(S)-3-(2-((((9H-fluoren-9-yl) methoxy) carbonyl)amino)-3-methoxy-3-oxopropyl)benzoate (15 g, 29.9 mmol, 84% yield).

### Step 6.

To a solution of tert-butyl(S)-3-(2-((((9H-fluoren-9-yl) methoxy) carbonyl)amino)-3-methoxy-3-oxopropyl)benzoate (18.00 g, 35.9 mmol) in THF (150 mL) and H₂O (150 mL) at RT, lithium hydroxide monohydrate (1.66 g, 39.5 mmol) was added. The reaction was stirred for 2 h at RT. The reaction was concentrated under reduced pressure to remove THF. In the basic medium the mixture was extracted with diethyl ether to remove the non polar impurities. The aqueous layer was acidified with aqueous citric acid solution and extracted with ethyl acetate. The organic layer was dried over sodium sulphate and concentrated under reduced to get the desired compound as a gummy solid which was further lyopholized to give off-white solids.

(A082E-559-01&05) the desired compound Lot 1: (S)-2-((((9H-fluoren-9-yl) methoxy) carbonyl)amino)-3-(3-(tert-butoxycarbonyl)phenyl) propanoic acid (11 g, 22.56 mmol, 62.9% yield). And lot 2: (S)-2-((((9H-fluoren-9-yl) methoxy) carbonyl)amino)-3-(3-(tert-butoxycarbonyl)phenyl) propanoic acid (5 g, 10.26 mmol, 28.6% yield). 7.86 (t, J=7.6 Hz, 2H), 7.75 (d, J=7.6 Hz, 1H), 7.66-7.59 (m, 2H), 7.52 (m, 2H), 7.41-7.37 (m, 3H), 7.31-7.24 (m, 2H), 4.21-4.16 (m, 4H), 3.17 (m, 1H), 2.96 (m, 1H), 1.53 (br, s. 9H). Analysis condition E: Retention time=3.865 min; ESI-MS(+) m/z [M−H]⁺: 486.2.

### Preparation of (S)-2-((((9H-fluoren-9-yl) methoxy) carbonyl)amino)-3-(m-tolyl) propanoic acid

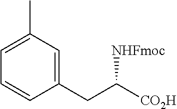

Compound was synthesized following the similar procedures of (S)-2-((((9H-fluoren-9-yl) methoxy) carbonyl)amino)-3-(3-(tert-butoxycarbonyl)phenyl) propanoic acid. Analysis condition E: Retention time=3.147 min; ESI-MS(+) m/z [M+H]⁺: 402.0. ¹H NMR (DMSO-d₆, 300 MHz) δ 7.88 (d, J=7.5 Hz, 2H), 7.64 (t, J=6.8 Hz, 2H), 7.44 (t, J=7.5 Hz, 2H), 7.36-7.28 (m, 2H), 7.18 (t, J=7.5 Hz, 1H), 7.09-7.02 (m, 3H), 4.24-4.17 (m, 4H), 3.21-3.04 (m, 1H), 2.89-2.81 (m, 1H), 2.26 (s, 3H) ppm.

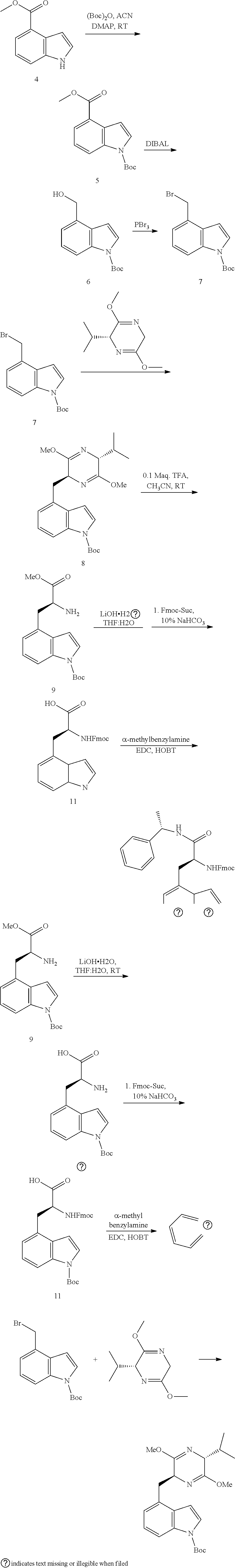

To a −78° C. cooled solution of (R)-2-isopropyl-3,6-dimethoxy-2,5-dihydropyrazine (29.7 g, 161 mmol) in THF (500 mL) was added n-butyllithium (77 mL, 193 mmol) slowly, and the reaction mass was stirred for 30 min. A solution of tert-butyl 4-(bromomethyl)-1H-indole-1-carboxylate (50 g, 161 mmol) in THF (500 mL) was added over a period of 10 min, and the reaction mass was stirred −78° C. for 1 h. The reaction was quenched with saturated ammonium chloride solution. The layers were separated and the aqueous layer was extracted with ethyl acetate (1000 mL) The combined organic layers were washed with brine, dried with sodium sulphate, and concentrated to get 75 g of the crude compound. The crude material of this batch was mixed with the crude compound of the 99597-878 for purification. Concentrated the product fractions to get 70 g of the required compound tert-butyl 4-(((2S,5R)-5-isopropyl-3,6-dimethoxy-2,5-dihydropyrazin-2-yl)methyl)-1H-indole-1-carboxylateas a colourless liquid.

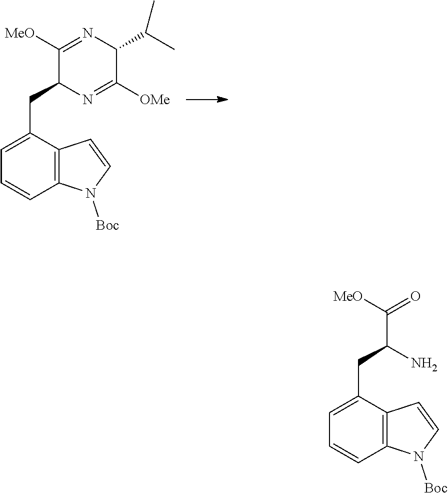 [SMILES: COC1=N[C@H](C(C)C)C(OC)=N[C@H]1Cc1cccc2c1ccn2C(=O)OC(C)(C)C ; COC(=O)[C@@H](N)Cc1cccc2c1ccn2C(=O)OC(C)(C)C]

To a 0° C. solution of tert-butyl 4-(((2S,5R)-5-isopropyl-3,6-dimethoxy-2,5-dihydropyrazin-2-yl)methyl)-1H-indole-1-carboxylate (70 g, 169 mmol) in acetonitrile (1600 mL) was added a solution of TFA (46 mL, 597 mmol) in water (500 mL), and then was stirred at room temperature for overnight. The solvent was removed and the aqueous layer was extracted with DCM (500 ml×3). The combined DCM layers were washed with brine solution, dried with sodium sulphate, and concentrated to give 54 g (89% HPLC purity). Analysis condition E: Retention time=2.658 min; ESI-MS(+) m/z [M+H]⁺: 305.2.

To a solution of tert-butyl(S)-4-(2-amino-3-methoxy-3-oxopropyl)-1H-indole-1-carboxylate (54 g, 170 mmol) in THF (1200 mL) was added a solution lithium hydroxide monohydrate (12.19 g, 509 mmol) in water (600 mL), then the reaction mass was stirred at room temperature for 30 min. THF was removed, and saturated 1N HCl solution was added to the residue to adjust pH to 5. The mixture was filtered and the solids were dried to get 45 g 73.2% 84% of the required compound. Analysis condition E: Retention time=1.524 min; ESI-MS(+) m/z [M+H]⁺: 305.2.

The mixture of (S)-2-amino-3-(1-(tert-butoxycarbonyl)-1H-indol-4-yl) propanoic acid (45 g, 129 mmol) and 10% sodium bicarbonate solution (750 mL) was stirred for 1 h, then added a solution of Fmoc-OSu (45.6 g, 135 mmol) in acetone (750 mL). The reaction was stirred at room temperature for 12 h. Acetone was removed completely. The reaction was cooled and then saturated citric acid solution was adjusted the pH to 5. The aqueous layer was extracted with ethyl acetate (500×3). The combined organic layers were washed with brine solution, dried with sodium sulphate, and concentrated to get 100 g of the crude compound. Purified the crude compound by ISCO, compound elutes with the 5% of methanol in chloroform. Concentrated the product fractions to get 40 g of the required compound. Dissolved in CH₂Cl₂ and concentrated to dryness to give(S)-2-((((9H-fluoren-9-yl) methoxy) carbonyl)amino)-3-(1-(tert-butoxycarbonyl)-1H-indol-4-yl) propanoic acid (40 g, 57.9% yield, 98% HPLC purity). Analysis condition E: Retention time=2.835 min; ESI-MS(+) m/z [M−H]⁺: 525.5. ¹H NMR (DMSO-d₆, 400 MHz), d 7.94 (d, J=8.0 Hz, 1H), 7.88 (d, J=7.6 Hz, 2H), 7.79 (d, J=7.6 Hz, 1H), 7.67 (d, J=3.6 Hz, 1H), 7.62 (d, J=7.6 Hz, 1H), 7.59 (d, J=7.6 Hz, 1H), 7.43-7.38 (m, 2H), 7.32-7.25 (m, 3H), 7.16 (d, J=7.2 Hz, 1H), 6.86 (d, J=8.0 Hz, 1H), 4.28-4.19 (m, 1H), 4.19-4.13 (m, 3H), 3.38-3.34 (m, 1H), 3.18-3.12 (m, 1H), 1.62 (s, 9H).

### Preparation ethyl(S)-5-((tert-butoxycarbonyl)amino)-2-(((S)-mesitylsulfinyl)amino)-3,3-dimethylpentanoate

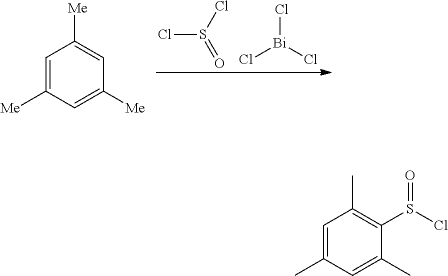

Step 1: The compound was synthesized using similar procedure described in reference: To a 1000-ml flask equipped with a septum inlet and magnetic stirring bar was added bismuth (III) chloride (5.25 g, 16.64 mmol). The flask was connected to an argon line and thionyl chloride (501 mL, 6864 mmol) were added by syringe. To the suspension was added mesitylene (100 g, 832 mmol). The flask was equipped with a condenser, connected to an oil bubbler and the reaction mixture was heated in an oil bath at 60° C. for 5 h. During this time the color of the solution became red-orange and HCl evolved from the solution. The reaction was monitored by LCMS. The flask was cooled in an ice bath and the excess of thionyl chloride was removed under reduced pressure yielding to an orange liquid. In order to remove the catalyst, 2000 mL of pentane were added, stirred and filtered through celite, and the bed was washed with pentane (2×500 mL). The organic phase was collected and evaporated under reduced pressure to give 2,4,6-trimethylbenzenesulfinic chloride (151 g, 745 mmol, 90% yield) as a pale yellow solid. The compound was taken to the next step without further purification. ¹H NMR (400 MHz, CDCl₃) δ 7.07-6.76 (m, 2H), 2.66 (s, 6H), 2.38-2.24 (m, 3H) ppm.

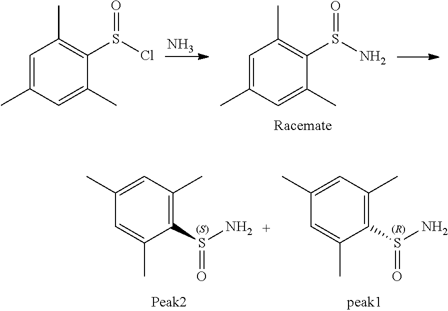

Step 2. The compound was synthesized using similar procedure described in reference: To a stirred solution of 2,4,6-trimethylbenzenesulfinic chloride (155 g, 765 mmol) in diethyl ether (1500 mL). After it had been cooled to −40° C. In a separate setup, (2 L multi neck RBF) taken in diethyl ether (900 mL) ammonia gas was bubbled 30 minutes at −40° C., this purged solution was added to above reaction mass at −40° C. After it had warmed to rt the reaction mixture was stirred for 2 hours and monitored by open access LCMS starting material was absent. The reaction was stirred at room temperature overnight according to given procedure. The reaction was monitored by TLC and open access LCMS, TLC wise starting material was absent. Workup: The reaction mixture was diluted with ethyl acetate (3000 mL) and washed with water (2000 ml), the organic layer was separated and the aqueous phase was again extracted with ethyl acetate (1×500 mL). The combined organic layer washed with brine (1×800 mL). The combined organic layer, dried (Na₂SO₄), filtered, and concentrated under reduced pressure to obtained (235 g) as a pale brown solid. The product (235 g) was recrystallized from 10% ethyl acetate/petroleum ether (500 mL), stirred, filtered, and dried to afford mesitylenesulphinamid (125 g) racemate as a white solid. The compound was submitted for the SFC method development. Two peaks were collected from SFC. The solvent was concentrated to give Peak-1 (Undesired): (R)-2,4,6-trimethylbenzenesulfinamide (51.6 g, 265 mmol, 34.6% yield) as a white colour solid. ¹H NMR (400 MHz, DMSO-d₆) δ 7.01-6.68 (m, 2H), 6.23-5.77 (m, 2H), 2.52-2.50 (m, 6H), 2.32-1.93 (m, 3H) and Peak-2 (desired): (S)-2,4,6-trimethylbenzenesulfinamide (51.6 g, 267 mmol, 35.0% yield) as a white colour solid. ¹H NMR (400 MHz, DMSO-d₆) δ 6.87 (s, 2H), 6.16-5.82 (m, 2H), 2.53-2.50 (m, 6H), 2.34-1.93 (m, 3H).

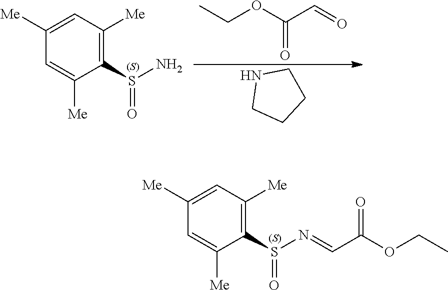

Step 3. The compound was synthesized using similar procedure described in reference: To a well stirred solution of (S)-2,4,6-trimethylbenzenesulfinamide (15.5 g, 85 mmol) in dichloromethane (235 mL) and 4A molecular sieves (84.5 g), was added ethyl 2-oxoacetate in toluene (25.9 mL, 127 mmol) and pyrrolidine (0.699 mL, 8.46 mmol). The reaction mixture was stirred at room temperature for overnight. The reaction was repeated and the two batches were combined together for work up. The reaction was mass was filtered throw the celite and the bed was washed with DCM. The solvents were removed under reduced pressure to obtained the crude (55 g) as a brownish color mass. The crude compound was purified by ISCO (Column size: 300 g silica column. Adsorbent: 60-120 silica mesh, Mobile phase: 40% EtOAc/Pet ether) and the product was collected at 15-20% of EtOAc. The fractions were concentrated to obtain ethyl (S,E)-2-((mesitylsulfinyl)imino) acetate (16.5 g, 57.4 mmol, 67.9% yield) as a colorless liquid. The compound slowly solidified as an off white solid. ¹H NMR (400 MHz, CDCl₃) δ=8.27 (s, 1H), 7.04-6.70 (m, 2H), 4.59-4.21 (m, 2H), 2.55-2.44 (m, 6H), 2.36-2.23 (m, 3H), 1.51-1.30 (m, 3H). 2.670 min. 268.2 (M+H).

Step 4. General procedure for the synthesis of TCNHPI redox-active esters as in reference ACIE: TCNHPI esters were prepared according to the previously reported general procedure (ACIE paper and references therein): A round-bottom flask or culture tube was charged with carboxylic acid (1.0 equiv), N-hydroxytetrachlorophthalimide (1.0-1.1 equiv) and DMAP (0.1 equiv). Dichloromethane was added (0.1-0.2 M), and the mixture was stirred vigorously. Carboxylic acid (1.0 equiv) was added. DIC (1.1 equiv) was then added dropwise via syringe, and the mixture was allowed to stir until the acid was consumed (determined by TLC). Typical reaction times were between 0.5 h and 12 h. The mixture was filtered (through a thin pad of Celite®, SiO₂, or frit funnel) and insed with additional CH₂Cl₂/Et₂O. The solvent was removed under reduced pressure, and purification of the crude mixture by column chromatography afforded the desired TCNHPI redox-active ester. If necessary, the TCNHPI redox-active ester could be further recrystallized from CH₂Cl₂/MeOH.

Step 5. 4,5,6,7-tetrachloro-1,3-dioxoisoindolin-2-yl-4-((tert-butoxycarbonyl)amino)-2,2-dimethylbutanoate was obtained as a white solid following General Procedure for the synthesis of TCNHPI redox-active esters on 5.00 mmol scale. Purification by column (silica gel, gradient from CH₂Cl₂ to 10:1 CH₂Cl₂:Et₂O) afforded 2.15 g (84%) of the title compound. ¹H NMR (400 MHz, CDCl₃): δ 4.89 (br s, 1H), 3.30 (q, J=7.0 Hz, 2H), 1.98 (t, J=7.6 Hz, 2H), 1.42 (s, 15H) ppm. ¹³C NMR (151 MHz, CDCl₃): δ 173.1, 157.7, 156.0, 141.1, 130.5, 124.8, 79.3, 40.8, 40.2, 36.8, 28.5, 25.2 ppm. HRMS (ESI-TOF): calc'd for C₁₉H₂₀C₁₄N₂NaO₆ [M+Na]⁺: 534.9968, found: 534.9973.

Step 6. Ethyl(S)-5-((tert-butoxycarbonyl)amino)-2-(((S)-mesitylsulfinyl)amino)-3,3-dimethylpentanoate was made using the General procedures for decarboxylative Amino acid synthesis in reference ACIE. A culture tube was charged with TCNHPI redox-active ester A (1.0 mmol), sulfinimine B (2.0 mmol), Ni(OAc)₂·4H₂O (0.25 mmol, 25 mol %), Zinc (3 mmol, 3 equiv). The tube was then evacuated and backfilled with argon (three times). Anhydrous NMP (5.0 mL, 0.2 M) was added using a syringe. The mixture was stirred overnight at rt. Then, the reaction mixture was diluted with EtOAc, washed with water, brine and dried over MgSO₄. Upon filtration, the organic layer was concentrated under reduced pressure (water bath at 30° C.), and purified by flash column chromatography (silica gel) to provide the product. Purification by column (2:1 hexanes: EtOAc) afforded 327.6 mg (72%) of the title compound ethyl(S)-5-((tert-butoxycarbonyl)amino)-2-(((S)-mesitylsulfinyl)amino)-3,3-dimethylpentanoate as a colorless oil. ¹H NMR (600 MHz, CDCl₃): δ 6.86 (s, 2H), 5.04 (d, J=10.1 Hz, 1H), 4.47 (s, 1H), 4.28-4.16 (m, 2H), 3.66 (d, J=10.1 Hz, 1H), 3.27-3.05 (m, 2H), 2.56 (s, 6H), 2.28 (s, 3H), 1.54-1.46 (m, 2H), 1.43 (s, 9H), 1.30 (t, J=7.2 Hz, 3H), 0.96 (s, 6H) ppm. ¹³C NMR (151 MHz, CDCl₃): δ 172.5, 155.9, 141.1, 137.9, 136.9, 131.0, 79.4, 65.5, 61.7, 38.8, 37.1, 36.5, 28.5, 23.9, 23.6, 21.2, 19.4, 14.3 ppm. HRMS (ESI-TOF): calc'd for C₂₃H₃₉N₂O₅S [M+H]⁺: 455.2574, found: 455.2569.

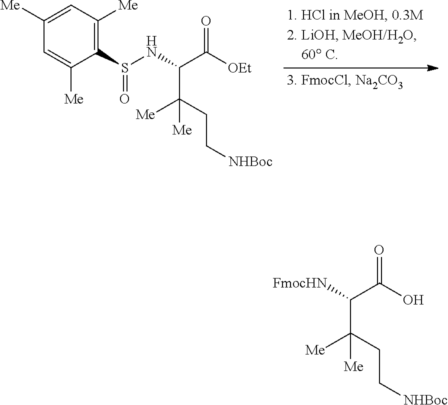

Step 7. 2-((((9H-fluoren-9-yl) methoxy) carbonyl)amino)-5-((tert-butoxycarbonyl)amino)-3,3-dimethylpentanoic acid: A culture tube was charged with ethyl(S)-5-((tert-butoxycarbonyl)amino)-2-(((S)-mesitylsulfinyl)amino)-3,3-dimethylpentanoate (0.5 mmol, 1.0 equiv), HCl (4.0 equiv) in MeOH (0.3 M) was added via syringe and the resulting mixture was stirred at RT for ca. 10 min (screened by TLC). After the reaction, Et₃N was added until pH=7 and the solvents were removed under reduced pressure. LiOH (2 equiv) in MeOH/H₂O (2:1, 0.04 M) was added to the crude mixture. The reaction was stirred at 60° C. overnight. On completion, HCl in MeOH (0.3 M) was added until pH=7 and the solvents were removed under reduced pressure. The crude mixture was dissolved in 9% aqueous Na₂CO₃ (5 mL) and dioxane (2 mL). It was slowly added at 0° C. to a solution of Fmoc-OSu (1.2 equiv) in dioxane (8 mL). The mixture was stirred at 0° C. for 1 h and then allowed to warm to rt. After 10 h, the reaction mixture was quenched with HCl (0.5 M), reaching pH 3, and then diluted with EtOAc. The aqueous phase was extracted with EtOAc (3×15 mL), and the combined organic layers were washed with brine, dried over Na₂SO₄, filtered, and the solvent was removed under reduced pressure. The crude mixture was then purified by flash column chromatography (silica gel, 2:1 hexanes: EtOAc) to afford the product ethyl(S)-5-((tert-butoxycarbonyl)amino)-2-(((S)-mesitylsulfinyl)amino)-3,3-dimethylpentanoate in 68% overall yield and 95% ee as a colorless oil. ¹H NMR (600 MHz, CDCl₃): δ 7.76 (d, J=7.5 Hz, 2H), 7.63-7.54 (m, 2H), 7.39 (td, J=7.3, 2.6 Hz, 2H), 7.33-7.28 (m, 2H), 5.50 (br s, 1H), 4.68 (br s, 1H), 4.45-4.43 (m, 1H), 4.38-4.35 (m, 1H), 4.30 (d, J=7.9 Hz, 1H), 4.21 (t, J=6.8 Hz, 1H), 3.27 (br s, 1H), 3.16 (br s, 1H), 1.63-1.50 (m, 2H), 1.43 (s, 9H), 1.09-0.76 (m, 6H) ppm. ¹³C NMR (151 MHz, CDCl₃): δ 185.8, 174.3, 156.5, 144.0, 143.9, 141.5, 127.9, 127.2, 125.24, 125.21, 120.2, 120.1, 79.8, 67.2, 60.9, 47.4, 39.2, 36.8, 29.9, 28.6, 23.9 ppm. HRMS (ESI-TOF): calc'd for C₂₇H₃₅N₂O₆ [M+H]⁺: 483.2490, found: 483.2489.

### Preparation of (S)-2-((((9H-fluoren-9-yl) methoxy) carbonyl)amino)-3-(4,4-difluorocyclohexyl) propanoic acid

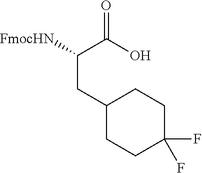

Final product was obtained following similar procedures of ethyl(S)-5-((tert-butoxycarbonyl)amino)-2-(((S)-mesitylsulfinyl)amino)-3,3-dimethylpentanoate. The synthesis afforded the desired(S)-2-((((9H-fluoren-9-yl) methoxy) carbonyl)amino)-3-(4,4-difluorocyclohexyl) propanoic acid (60 mg, 0.14 mmol, 27.9% yield) as a white solid after purification by reverse phase HPLC. ¹H NMR (500 MHz, CDCl₃) δ 7.79 (br d, J=7.5 Hz, 2H), 7.61 (br s, 2H), 7.43 (s, 2H), 7.36-7.31 (m, 2H), 5.24-5.06 (m, 1H), 4.57-4.36 (m, 3H), 4.29-4.16 (m, 1H), 2.19-1.99 (m, 2H), 1.97-1.18 (m, 9H).

### Preparation of (2S)-5-(tert-butoxy)-2-({[(9H-fluoren-9-yl) methoxy]carbonyl}amino)-3,3-dimethyl-5-oxopentanoic acid

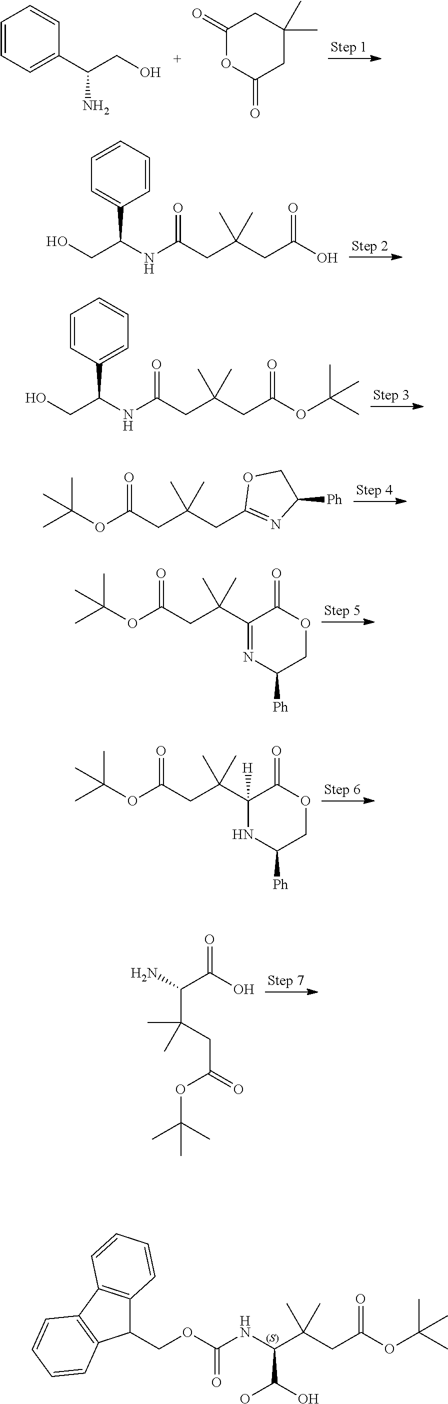

### Step 1

A solution of 4,4-dimethyldihydro-2H-pyran-2,6 (3H)-dione (8.29 g, 58.3 mmol) in dry toluene (100 mL) was slowly added to a solution of (R)-2-amino-2-phenylethan-1-ol (10 g, 72.9 mmol) in dry toluene (100 mL) and CH₂Cl₂ (20 mL) at room temperature. The reaction mixture was then heated to 60° C. and reacted for 12 h. It was cooled to room temperature until a white solid was formed. The solid was filtered and washed with 1:1 EtOAc/CH₂Cl₂ to afford the crude desired compound (R)-5-((2-hydroxy-1-phenylethyl)amino)-3,3-dimethyl-5-oxopentanoic acid (11.9 g, 41.0 mmol, 56.2% yield) without further purification. ¹H NMR (300 MHz, DMSO-d₆) δ 8.41 (br d, J=7.9 Hz, 1H), 7.44-7.32 (m, 2H), 7.32-7.27 (m, 4H), 7.26-7.18 (m, 1H), 4.89-4.80 (m, 1H), 4.14-3.98 (m, 1H), 3.63-3.43 (m, 3H), 2.27-2.18 (m, 4H), 2.08 (s, 1H), 1.99 (s, 1H), 1.17 (t, J=7.2 Hz, 1H), 1.00 (d, J=4.5 Hz, 6H), 0.92 (s, 1H).

### Step 2

(R)-5-((2-Hydroxy-1-phenylethyl)amino)-3,3-dimethyl-5-oxopentanoic acid (12 g, 43.0 mmol) was dissolved in a solution of benzyltrimethylammonium chloride (8.93 g, 48.1 mmol) in DMA (250 mL). K₂CO₃ (154 g, 1117 mmol) was added to the above solution followed by the addition of 2-bromo-2-methylpropane (235 mL, 2091 mmol). The reaction mixture was stirred at 55° C. for 24 h. The reaction mixture was then diluted with EtOAc (100 mL), washed with H₂O (50 mL×3), and brine (50 mL). The organic phase was dried over Na₂SO₄, concentrated under vacuo, and purified by flash column chromatography on silica gel (CH₂Cl₂/MeOH, 15:1) to give tert-butyl (R)-5-((2-hydroxy-1-phenylethyl)amino)-3,3-dimethyl-5-oxopentanoate (6.0 g, 17.89 mmol, 41.6% yield). Analytical LC/MS Condition M: 1.96 min, 336.3 [M+H]⁺. ¹H NMR (300 MHz, DMSO-d₆) d=8.14 (br d, J=8.3 Hz, 1H), 7.33-7.25 (m, 4H), 7.25-7.17 (m, 1H), 4.90-4.77 (m, 2H), 3.52 (br t, J=5.7 Hz, 2H), 3.34 (s, 1H), 2.94 (s, 1H), 2.78 (s, 1H), 2.20 (d, J=14.0 Hz, 4H), 1.97 (d, J=9.8 Hz, 2H), 1.41-1.31 (m, 9H), 1.00 (d, J=1.1 Hz, 6H).

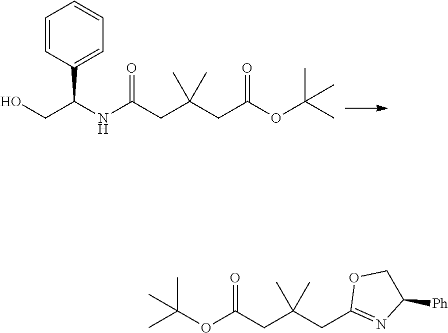

### Step 3

tert-Butyl (R)-5-((2-hydroxy-1-phenylethyl)amino)-3,3-dimethyl-5-oxopentanoate (6 g, 17.89 mmol) and 2,3-dichloro-5,6-dicyano-p-benzoquinone (6.09 g, 26.8 mmol) was dissolved in dry dichloromethane (70 mL) under Ar. Triphenylphosphine (7.04 g, 26.8 mmol) was added to the above solution. The reaction mixture was stirred at room temperature for 2 h. The crude product was then concentrated under vacuo and purified by flash column chromatography on silica gel (EtOAc/Hexanes, 1:5) to give tert-butyl (R)-3,3-dimethyl-4-(4-phenyl-4,5-dihydrooxazol-2-yl) butanoate (5.6 g, 17.64 mmol, 99% yield). ESI-MS(+) m/z: 318.3 [M+H]⁺. ¹H NMR (300 MHz, DMSO-d₆) d=7.41-7.18 (m, 5H), 5.18 (t, J=9.1 Hz, 1H), 4.59 (dd, J=8.7, 10.2 Hz, 1H), 3.94-3.85 (m, 1H), 3.94-3.85 (m, 1H), 3.95-3.84 (m, 1H), 4.10-3.84 (m, 1H), 2.43-2.22 (m, 4H), 1.40 (s, 9H), 1.09 (d, J=1.9 Hz, 6H).

### Step 4

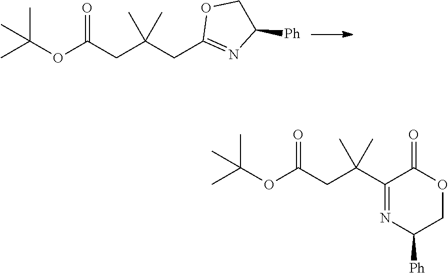 [SMILES: CC(C)(CC(=O)OC(C)(C)C)CC1=N[C@H](c2ccccc2)CO1 ; CC(C)(C)OC(=O)CC(C)(C)C1=N[C@H](c2ccccc2)COC1=O]

A solution of tert-butyl (R)-3,3-dimethyl-4-(4-phenyl-4,5-dihydrooxazol-2-yl) butanoate (5.6 g, 17.64 mmol) in EtOAc (250 mL) was added selenium dioxide (4.89 g, 44.1 mmol) and refluxed for 2 h. The reaction mixture was then cooled to room temperature and stirred for 12 h. The crude product was then concentrated in vacuo and purified by flash column chromatography on silica gel (EtOAc/Hexanes, 1:7) to afford tert-butyl (R)-3-methyl-3-(2-oxo-5-phenyl-5,6-dihydro-2H-1,4-oxazin-3-yl) butanoate (1.3 g, 3.92 mmol, 22.23% yield) as a colorless liquid. ESI-MS(+) m/z: 332.2 [M+H]⁺. ¹H NMR (CDCl₃) δ 1.37 (s, 3H), 1.42 (s, 9H), 1.44 (s, 3H), 2.59 (d, J=15.5 Hz, 1H), 3.12 (d, J=15.5 Hz, 1H), 4.32 (t, J=11.1 Hz, 1H), 4.47 (dd, J=4.3 Hz, J=6.7 Hz, 1H), 4.80 (dd, J=4.3 Hz, J=6.7 Hz, 1H), 7.35-7.39 (m, 5H). 13C NMR (CD3Cl) δ 26.40, 27.29, 28.00, 40.84, 45.94, 59.72, 70.88, 80.63, 127.13, 127.92, 128.65, 137.58, 155.07, 167.46, 171.95.

### Step 5

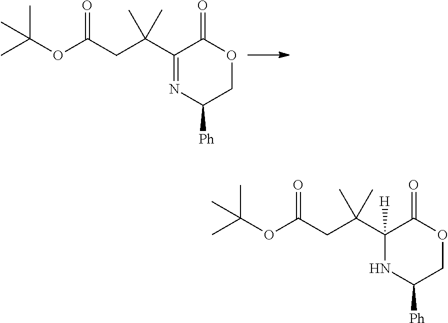 [SMILES: CC(C)(C)OC(=O)CC(C)(C)C1=N[C@H](c2ccccc2)COC1=O ; CC(C)(C)OC(=O)CC(C)(C)[C@@H]1N[C@H](c2ccccc2)COC1=O]

Platinum(IV) oxide monohydrate (130 mg, 0.530 mmol) was added to a solution of tert-butyl (R)-3-methyl-3-(2-oxo-5-phenyl-5,6-dihydro-2H-1,4-oxazin-3-yl) butanoate (1.3 g, 3.92 mmol) in methanol (50 mL). The reaction flask was purged with H₂ (3×) and stirred under H₂ for 24 h. After venting the vessel, the reaction mixture was filtered through Celite, and the filtrate was washed with EtOAc. The crude product was concentrated under vacuo and purified by flash column chromatography on silica gel (EtOAc/Hexanes, 1:8) to give tert-butyl 3-methyl-3-((3S,5R)-2-oxo-5-phenylmorpholin-3-yl) butanoate (1.2 g, 3.33 mmol, 85% yield). ¹H NMR (300 MHz, DMSO-d₆) δ 7.52-7.42 (m, 2H), 7.41-7.26 (m, 3H), 4.30-4.20 (m, 2H), 4.13 (d, J=10.6 Hz, 1H), 3.80 (d, J=7.6 Hz, 1H), 3.07-2.98 (m, 1H), 2.47 (br s, 1H), 2.27 (d, J=13.6 Hz, 1H), 1.43-1.35 (m, 9H), 1.17-1.07 (m, 5H).

### Step 6.

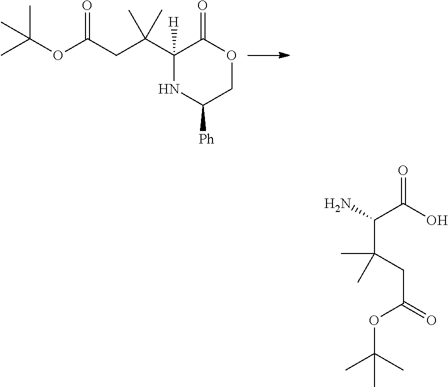 [SMILES: CC(C)(C)OC(=O)CC(C)(C)[C@@H]1N[C@H](c2ccccc2)COC1=O ; CC(C)(C)OC(=O)CC(C)(C)[C@H](N)C(=O)O]

Pearlman's catalyst Pd(OH)₂ on carbon (1.264 g, 1.799 mmol, 20% w/w) was added to a solution of tert-butyl 3-methyl-3-((3S,5R)-2-oxo-5-phenylmorpholin-3-yl) butanoate (1.2 g, 3.60 mmol) in methanol (50 mL)/water (3.13 mL)/TFA (0.625 mL) (40:2.5:0.5, v/v/v). The vessel was purged with H₂ and stirred under H₂ for 24 h. After venting the vessel, the reaction mixture was filtered through Celite, and the filtrate was washed with MeOH. The crude product ((S)-2-amino-5-(tert-butoxy)-3,3-dimethyl-5-oxopentanoic acid (0.83 g, 3.59 mmol, 100% yield)) was concentrated under vacuo. This crude was taken for the next step without further purification. Analytical LC/MS Condition M: 1.13 min, 232.2 [M+H]⁺.

### Step 7.

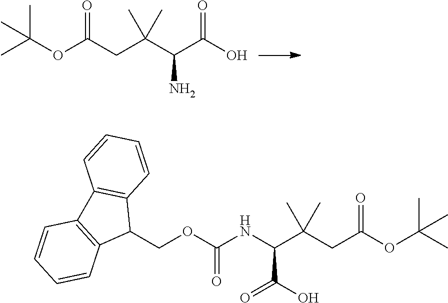 [SMILES: CC(C)(C)OC(=O)CC(C)(C)[C@H](N)C(=O)O ; CC(C)(C)OC(=O)CC(C)(C)[C@H](NC(=O)OCC1c2ccccc2-c2ccccc21)C(=O)O]

The crude product(S)-2-amino-5-(tert-butoxy)-3,3-dimethyl-5-oxopentanoic acid (1 g, 4.32 mmol) dissolved in water (30 mL). Na₂CO₃ (0.916 g, 8.65 mmol) was then added to the above solution. To this solution, fmoc n-hydroxysuccinimide ester (1.458 g, 4.32 mmol) in dioxane (30 mL) was added drop wise at 0° C. and stirred at room temperature for 16 h. The reaction mixture was acidified to pH ˜2 by 1N HCl and extracted with EtOAc (50 mL×3), dried over Na₂SO₄, concentrated under vacuo and purified by flash column chromatography on silica gel (EtOAc/petroleum ether, 35 to 39%) to give(S)-2-((((9H-fluoren-9-yl) methoxy) carbonyl)amino)-5-(tert-butoxy)-3,3-dimethyl-5-oxopentanoic acid (0.73 g, 1.567 mmol, 36.2% yield) as a white solid. LCMS, Analytical LC/MS Condition E, MS (ESI) t_{R}=2.135 min, m/z 452.2 [M−H]⁻. ¹H NMR (400 MHz, DMSO-d₆) δ 12.78-12.64 (m, 1H), 7.90 (d, J=7.5 Hz, 2H), 7.77 (dd, J=4.5, 7.0 Hz, 2H), 7.65 (br d, J=9.5 Hz, 1H), 7.46-7.39 (m, 2H), 7.37-7.29 (m, 2H), 4.32-4.15 (m, 4H), 2.39-2.31 (m, 1H), 2.30-2.21 (m, 1H), 1.39 (s, 9H), 1.12-1.00 (m, 6H).

### Preparation of (2S)-2-({[(9H-fluoren-9-yl) methoxy]carbonyl}amino)-3-(morpholin-4-yl) propanoic acid

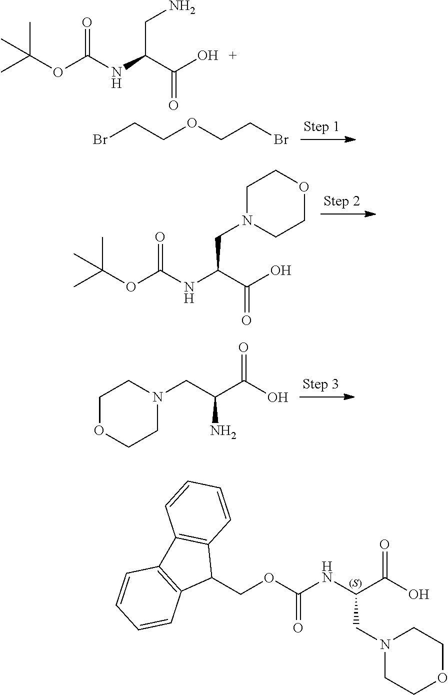

### Step 1

In a 2-L multi-necked round-bottomed flask fitted with a thermo pocket was added(S)-3-amino-2-((tert-butoxycarbonyl)amino) propanoic acid (50 g, 245 mmol), dioxane (500 mL), followed by 1-bromo-2-(2-bromoethoxy) ethane (30.8 mL, 245 mmol) at rt. NaOH (367 mL, 734 mmol) solution was added and the resulting yellow clear solution was heated to 110° C. (external temperature, 85° C. internal temperature) for 12 h. An aliquot of clear solution was subjected to LCMS (Polar method) which showed completion, and then the dioxane was evaporated to get light red solution which was acidified to pH 3. The resulting mixture was concentrated under high vacuum pump (˜4 mbar) at 60° C. to get(S)-2-((tert-butoxycarbonyl)amino)-3-morpholinopropanoic acid (67 g, 244 mmol, 100% yield) pale yellow solid. Analytical LC/MS Condition M: 0.56 min, 275.2 [M+H]⁺.

### Step 2

To a stirred suspension of (S)-2-((tert-butoxycarbonyl)amino)-3-morpholinopropanoic acid (100 g, 365 mmol) in dioxane (400 mL) at 0-5° C. was added HCl in dioxane (911 mL, 3645 mmol) slowly over 20 min. The resulting mixture was stirred at rt for 12 h. The volatile was evaporated to get pale yellow sticky crude(S)-2-amino-3-morpholinopropanoic acid (16 g, 92 mmol, 97% yield), This crude was taken for next step without further purification. MS (ESI) m/z 175.2 [M+H]⁺.

### Step 3

The crude product(S)-2-amino-3-morpholinopropanoic acid (11 g, 63.1 mmol) dissolved in water (250 mL). Na₂CO₃ (13.39 g, 126 mmol) was then added to the above solution. To this solution, Fmoc N-hydroxysuccinimide ester (21.30 g, 63.1 mmol) was added dropwise at 0 C and stirred at room temperature for 16 h. The reaction mixture was acidified to pH ˜2 by 1N HCl and extracted with EtOAc (500 mL×3), dried over Na₂SO₄, concentrated under vacuo, and purified by flash column chromatography on silica gel (petroleum ether/EtOAc, 0-100% then MeOH/CHCl₃ 0-15%) to get (S)-2-((((9H-fluoren-9-yl) methoxy) carbonyl)amino)-3-morpholinopropanoic acid (23 g, 55.9 mmol, 89% yield) as a brown solid. Analytical LC MS Condition E: 1.43 min, 397.2 [M+H]⁺. ¹H NMR (400 MHz, METHANOL-d₄) δ 7.78 (br d, J=7.5 Hz, 2H), 7.71-7.57 (m, 2H), 7.42-7.34 (m, 2H), 7.34-7.26 (m, 2H), 4.71 (br s, 1H), 4.54-4.32 (m, 2H), 4.29-4.17 (m, 1H), 3.90 (br s, 4H), 3.76-3.62 (m, 1H), 3.58-3.47 (m, 1H), 3.41 (br s, 2H), 3.36-3.32 (m, 2H), 3.31-3.26 (m, 1H).

### Preparation of (2S,3S)-3-{[(tert-butoxy) carbonyl]amino}-2-({[(9H-fluoren-9-yl)methoxy]carbonyl}amino) butanoic acid

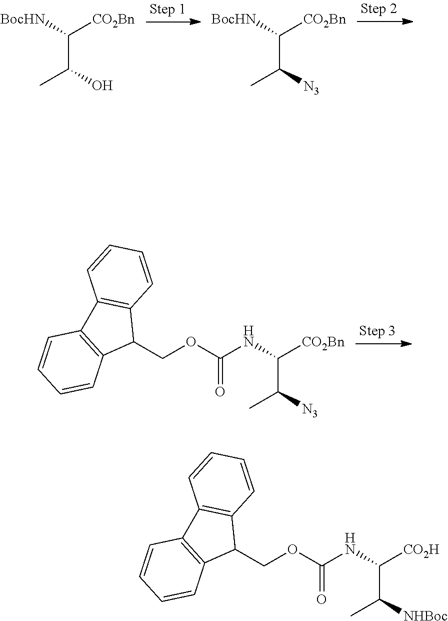 [SMILES: C[C@@H](O)[C@H](NC(=O)OC(C)(C)C)C(=O)OCc1ccccc1 ; C[C@H](N=[N+]=[N-])[C@H](NC(=O)OC(C)(C)C)C(=O)OCc1ccccc1 ; C[C@H](N=[N+]=[N-])[C@H](NC(=O)OCC1c2ccccc2-c2ccccc21)C(=O)OCc1ccccc1 ; C[C@H](NC(=O)OC(C)(C)C)[C@H](NC(=O)OCC1c2ccccc2-c2ccccc21)C(=O)O]

### Step 1

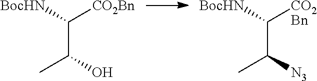 [SMILES: C[C@@H](O)[C@H](NC(=O)OC(C)(C)C)C(=O)OCc1ccccc1 ; C[C@H](N=[N+]=[N-])[C@H](NC(=O)OC(C)(C)C)C(=O)OCc1ccccc1]

To a solution of the benzyl (tert-butoxycarbonyl)-L-threoninate (22 g, 71.1 mmol) in CH₂Cl₂ (600 mL) at −78° C. was sequentially added trifluoromethanesulfonic anhydride (24.08 g, 85 mmol) dropwise and then 2,6-lutidine (10.77 mL, 92 mmol) slowly. After stirring at the same temperature for 1.5 h and monitoring by TLC (Hex: EtOAc 8:2), tetrabutylammonium azide (50.6 g, 178 mmol) was added in portions. After stirring at −78° C. for 1 h, the cooling bath was removed and the reaction mixture was allowed to reach 23° C. for 1.5 h. The reaction was repeated. A saturated aqueous solution of NaHCO₃ was added, and the aqueous phase extracted with EtOAc. The crude product was purified by flash chromatography over silica gel (Hex: EtOAc 95:5 a 9:1) to give benzyl (2S,3S)-3-azido-2-((tert-butoxycarbonyl)amino) butanoate (20 g, 59.8 mmol, 84% yield) as colorless liquid. Analytical LC/MS Condition E: 3.13 min, 333.2 [M−H]⁻.

### Step 2

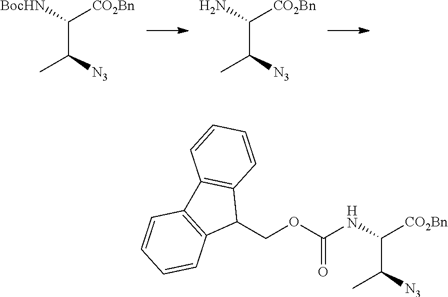 [SMILES: C[C@H](N=[N+]=[N-])[C@H](NC(=O)OC(C)(C)C)C(=O)OCc1ccccc1 ; C[C@H](N=[N+]=[N-])[C@H](N)C(=O)OCc1ccccc1 ; C[C@H](N=[N+]=[N-])[C@H](NC(=O)OCC1c2ccccc2-c2ccccc21)C(=O)OCc1ccccc1]

A solution of benzyl (2S,3S)-3-azido-2-((tert-butoxycarbonyl)amino) butanoate (20 g, 59.8 mmol), dichloromethane (300 mL) and TFA (50 mL, 649 mmol) was stirred for 2 h at 23° C. and then evaporated to dryness to give the corresponding amine. The above amine was redisolved in water (200 mL) and tetrahydrofuran (200 mL). At 0° C., DIPEA (11.49 mL, 65.8 mmol) was added followed by Fmoc chloride (17.02 g, 65.8 mmol). The mixture was warmed up to rt and stirred for 3 h. It was extracted with EtOAc and washed with 0.5 M HCl solution and then brine solution. It was concentrated to get crude liquid. The above crude was purifirf by silica gel column chromatography. The product was eluted at 20% EtOAc in petroleum ether. The fractions were concentrated to get benzyl (2S,3S)-2-((((9H-fluoren-9-yl) methoxy) carbonyl)amino)-3-azidobutanoate (23 g, 50.4 mmol, 84% yield) as a colorless liquid. Analytical LC/MS Condition E: 3.70 min, 479.3 [M+Na]⁺.

### Step 3.

To a multi-neck round-bottled flask was charged benzyl (2S,3S)-2-((((9H-fluoren-9-yl) methoxy) carbonyl)amino)-3-azidobutanoate (40 g, 88 mmol) in tetrahydrofuran (1200 mL). Pd/C (9.32 g, 8.76 mmol) was added under nitrogen and the reaction was stirred under hydrogen for 12 h. Sodium bicarbonate (11.04 g, 131 mmol) in water 6 (mL) was added followed by Boc-anhydride (30.5 mL, 131 mmol). The mixture was stirring under nitrogen for 12 h. The reaction mass was filtered through cellite bed, washed the bed with THF/Water mixture. The mother liquid was concentrated and washed with EtOAc. Then pH of water layer was adjusted to 7-6 using 1.5 N HCl solution. The resulting white solid was extracted with ethylacetate. The above reaction was repeated three more times. The combined organics were washed with water and brine solution, dried over sodium sulphate, and concentrated to afford (2S,3S)-2-((((9H-fluoren-9-yl) methoxy) carbonyl)amino)-3-((tert-butoxycarbonyl)amino) butanoic acid as a white solid (28 g). This was mixed with a previously obtained batch (8 g) in DCM (200 mL). n-Hexane (1 L) was added to the above solution and sonicated for 2 min. The solids were filtered, rinsed with hexanes and dried overnight to give (2S,3S)-2-((((9H-fluoren-9-yl) methoxy) carbonyl)amino)-3-((tert-butoxycarbonyl)amino) butanoic acid (36 g, 81 mmol, 92% yield) as a white powder. Analytical LC/MS Condition E: 1.90 min, 439.2 [M−H]⁻. ¹H NMR (400 MHz, DMSO-d₆) δ 7.90 (d, J=7.6 Hz, 2H), 7.75 (d, J=7.2 Hz, 2H), 7.43 (t, J=7.2 Hz, 2H), 7.34 (t, J=Hz, 6.71 (br. d. J=7.6 Hz, 1H), 4.29-4.26 (m, 2H), 4.25-4.21 (m, 1H), 3.94-3.90 (m, 1H), 1.37 (s, 9H), 1.02 (d, J=6.8 Hz, 3H). ¹³C NMR (101 Hz, DMSO-d₆) δ 171.9, 156.3, 154.8, 143.7, 140.6, 127.6, 127.0, 125.3, 120.0, 77.7, 65.8, 57.8, 47.0, 46.6, 28.2, 16.2.

### Preparation of (S)-2-((((9H-fluoren-9-yl) methoxy) carbonyl)amino)-2-(1-(((tert-butoxycarbonyl)amino)methyl)cyclopropyl) acetic acid

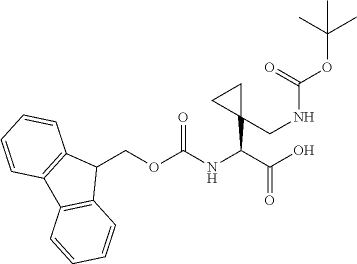 [SMILES: CC(C)(C)OC(=O)NCC1([C@H](NC(=O)OCC2c3ccccc3-c3ccccc32)C(=O)O)CC1]

Final product was obtained following similar procedures of ethyl(S)-5-((tert-butoxycarbonyl)amino)-2-(((S)-mesitylsulfinyl)amino)-3,3-dimethylpentanoate. The synthesis afforded the desired product (0.65 g, 22% yield) as a white solid after purification by flash column chromatography (Red Sep, 40 g, SiO₂, 35 to 40% EtOAc: hexanes (compound ELSD active)). Analytical LC/MS Condition E: 2.04 min, 465.2 [M−H]⁻. ¹H NMR (300 MHz, DMSO-d₆) δ 7.90 (d, J=7.6 Hz, 2H), 7.71 (m, 3H), 7.47-7.27 (m, 2H), 6.98-6.71 (m, 2H), 4.30-4.17 (m, 3H), 3.94-3.82 (m, 1H), 3.20-2.90 (m, 2H), 1.44-1.30 (m, 9H), 0.48 (br s, 4H).

### Preparation of (S)-2-((((9H-fluoren-9-yl) methoxy) carbonyl)amino)-2-(1-(tert-butoxycarbonyl) azetidin-3-yl) acetic acid

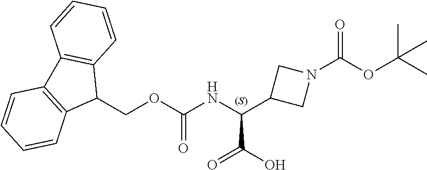

Final product was obtained following similar procedures of ethyl(S)-5-((tert-butoxycarbonyl)amino)-2-(((S)-mesitylsulfinyl)amino)-3,3-dimethylpentanoate. The synthesis afforded the desired product (2.66 g, 20% yield) as a slightly tan solid after purification by reverse-phase HPLC. Analytical LC/MS Condition E: 1.87 min, 467.2 [M−H]⁻. ¹H NMR (400 MHz, DMSO-d₆) δ 7.89 (d, J=7.6 Hz, 2H), 7.69 (m, 2H), 7.41 (t, J=7.2 Hz, 2H), 7.34-7.31 (m, 2H), 6.71 (br. d. J=7.6 Hz, 1H), 4.29-4.23 (m, 3H), 3.77-3.70 (m, 5H), 2.80 (m, 1H), 1.36 (s, 9H).

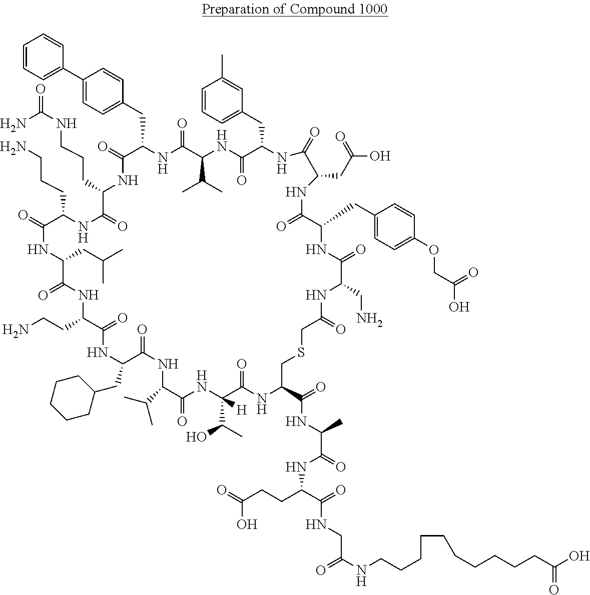 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

To a 45-mL polypropylene solid-phase reaction vessel was added 2-Chlorotrityl resin pre-loaded with 11-((((9H-fluoren-9-yl) methoxy) carbonyl)amino) undecanoic acid (90 mg, 0.050 mmol), and the reaction vessel was placed on the Prelude peptide synthesizer. The following procedures were then performed sequentially: “Prelude Single-Coupling Procedure” was followed; “Prelude Single-Coupling Procedure” was followed with Fmoc-Gly-OH; “Prelude Single-Coupling Procedure” was followed with Fmoc-Glu (tBu)-OH; “Prelude Single-Coupling Procedure” was followed with Fmoc-Ala-OH; “Prelude Single-Coupling Procedure” was followed with Fmoc-Cys(Trt)-OH; “Prelude Single-Coupling Procedure” was followed with Fmoc-Thr(tBu)-OH; “Prelude Single-Coupling Procedure” was followed with Fmoc-Val-OH; “Prelude Single-Coupling Procedure” was followed with Fmoc-Cha-OH; “Prelude Single-Coupling Procedure” was followed with Fmoc-Dab(Boc)-OH; “Prelude Single-Coupling Procedure” or “Prelude Double-Coupling Procedure” was followed with Fmoc-D-Leu-OH; “Prelude Single-Coupling Procedure” or “Prelude Double-Coupling Procedure” was followed with Fmoc-Orn(Boc)-OH; “Prelude Single-Coupling Procedure” was followed with Fmoc-Cit-OH; “Prelude Single-Coupling Procedure” was followed with Fmoc-Bip-OH; “Prelude Single-Coupling Procedure” was followed with Fmoc-Val-OH; “Prelude Single-Coupling Procedure” was followed with Fmoc-Phe (3-Me)-OH; “Prelude Single-Coupling Procedure” was followed with Fmoc-Asp(tBu)-OH; “Prelude Single-Coupling Procedure” was followed with Tyr(CH₂COOtBu)-OH; “Prelude Single-Coupling Procedure” was followed with Dap(Boc)-OH; “Prelude Chloroacetic Anhydride coupling procedure” was followed; “Prelude Final rinse and dry procedure” was followed; “Global Deprotection Method A” was followed; “Cyclization Method A” was followed.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 18-58% B over 20 minutes, then a 4-minute hold at 100% B; Flow: 20 mL/min. Fractions containing the desired product were combined and dried via centrifugal evaporation. The yield of the product was 19.3 mg, and its estimated purity by LCMS analysis was 92%.

$$Retention time = 1.5 min ; \\ ESI - MS ( + ) m / {z [ M + 2 H ]}^{2 +} : 1174. . \qquad Analysis condition A$$

$$Retention time = 1.56 min ; \\ ESI - MS ( + ) m / {z [ M + 2 H ]}^{2 +} : 782.9 . \qquad Analysis condition B$$

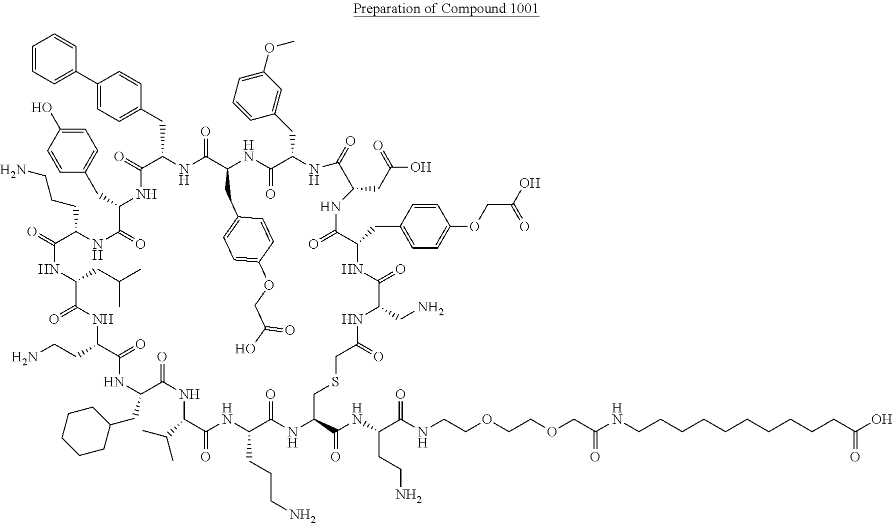 [SMILES: COc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

To a 45-mL polypropylene solid-phase reaction vessel was added 2-chlorotrityl resin pre-loaded with 11-((((9H-fluoren-9-yl) methoxy) carbonyl)amino) undecanoic acid on a 50 μmol scale, and the reaction vessel was placed on the Symphony peptide synthesizer. The following procedures were then performed sequentially: “Symphony Resin-swelling procedure” was followed; “Symphony Single-coupling procedure” was followed with FmocNH—CH₂CH₂OCH₂CH₂OCH₂COOH; “Symphony Single-Coupling Procedure” was followed with Fmoc-Dab(Boc)-OH; “Symphony Single-Coupling Procedure” was followed with Fmoc-Cys(Trt)-OH; “Symphony Single-Coupling Procedure” was followed with Fmoc-Orn(Boc)-OH; Symphony Single-Coupling Procedure” was followed with Fmoc-Val-OH; “Symphony Single-Coupling Procedure” was followed with Fmoc-Cha-OH; “Symphony Single-Coupling Procedure” was followed with Fmoc-Dab(Boc)-OH; “Symphony Single-Coupling Procedure” was followed with Fmoc-D-Leu-OH; “Symphony Single-Coupling Procedure” was followed with Fmoc-Orn(Boc)-OH; “Symphony Single-Coupling Procedure” was followed with Fmoc-Tyr(tBu)-OH; “Symphony Double-Coupling Procedure” was followed with Fmoc-Bip-OH; “Symphony Single-Coupling Procedure” was followed with Fmoc-Tyr(CH₂COOtBu)-OH; “Symphony Single-Coupling Manual Addition procedure” was followed with Fmoc-Phe (3-OMe)-OH; “Symphony Single-Coupling Procedure” was followed with Fmoc-Asp(tBu)-OH; “Symphony Single-Coupling Procedure” was followed with Fmoc-Tyr(CH₂COO/Bu) OH; “Symphony Single-Coupling Procedure” was followed with Fmoc-Dap(Boc)-OH; “Symphony Chloroacetic Anhydride Coupling Procedure” was followed; “Global Deprotection Method A” was followed; “Cyclization Method” was followed.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 45 mL/min; Column: Temperature: 25 C. Fraction collection was triggered by MS signals. Fractions containing the desired product were combined and dried via centrifugal evaporation. The yield of the product was 7.8 mg, and its estimated purity by LCMS analysis was 97%.

$$Retention time = 1.54 min ; \\ ESI - MS ( + ) m / {z [ M + 3 H ]}^{3 +} : 831.2 . \qquad Analysis condition A$$

$$Retention time = 1.55 min ; \\ ESI - MS ( + ) m / {z [ M + 2 H ]}^{3 +} : 831.2 . \qquad Analysis condition B$$

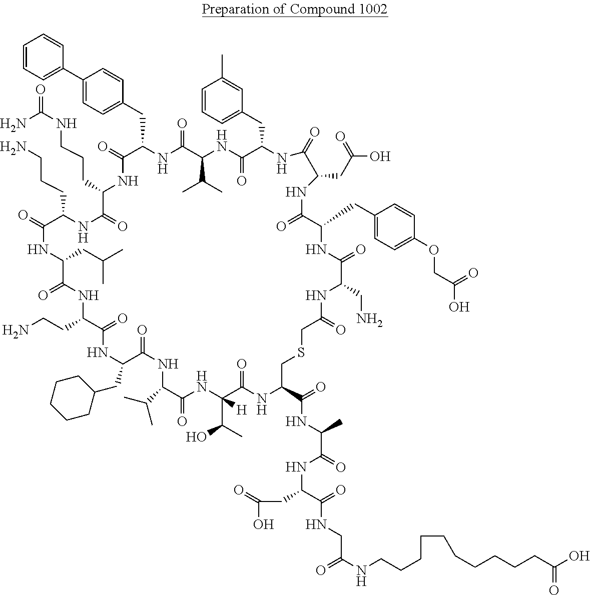 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)O)C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

To a 45-mL polypropylene solid-phase reaction vessel was added 2-chlorotrityl resin pre-loaded with 11-((((9H-fluoren-9-yl) methoxy) carbonyl)amino) undecanoic acid on a 50 μmol scale, and the reaction vessel was placed on the Symphony X peptide synthesizer. The following procedures were then performed sequentially: “Symphony X Resin-swelling procedure” was followed; “Symphony X Single-coupling procedure” was followed with Fmoc-Gly-OH; “Symphony X Single-coupling procedure” was followed with Fmoc-Asp(OtBu)-OH; “Symphony X Single-coupling procedure” was followed with Fmoc-Ala-OH; “Symphony X Single-coupling procedure” was followed with Fmoc-Cys(Trt)-OH; “Symphony X Single-coupling procedure” was followed with Fmoc-Thr(tBu)-OH; “Symphony X Single-coupling procedure” was followed with Fmoc-Val-OH; “Symphony X Single-coupling procedure” was followed with Fmoc-Cha-OH; “Symphony X Single-coupling procedure” was followed with Fmoc-Dab(OtBu)-OH; “Symphony X Single-coupling procedure” or “Symphony Double-coupling procedure” was followed with Fmoc-D-Leu-OH; “Symphony X Single-coupling procedure” or “Symphony Double-coupling procedure” was followed with Fmoc-Orn-OH; “Symphony X Single-coupling procedure” or “Symphony Double-coupling procedure” was followed with Cit-OH; “Symphony X double-coupling procedure” was followed with Fmoc-Bip-OH; “Symphony X single-coupling procedure” was followed with Fmoc-Val-OH; “Symphony X single-coupling procedure” was followed with Fmoc-Phe (3-Me)-OH; “Symphony X single-coupling procedure” was followed with Fmoc-Asp(tBu)-OH; “Symphony Single-coupling procedure” was followed with Fmoc-Tyr(CH₂COOtBu)-OH; “Symphony X Single-coupling procedure” was followed with Fmoc-Dap(Boc)-OH; “Symphony X Chloroacetic Anhydride coupling procedure” was followed; “Global Deprotection Method A” was followed; “Cyclization Method” was followed.

The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 8% B, 8-48% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 5.4 mg, and its estimated purity by LCMS analysis was 95%.

$$Retention time = 1.44 min ; \\ ESI - MS ( + ) m / {z [ M + 2 H ]}^{2 +} : 1098.1 . \qquad Analysis condition A$$

$$Retention time = 1.94 min ; \\ ESI - MS ( + ) m / {z [ M + 2 H ]}^{2 +} : 1097.9 . \qquad Analysis condition B$$

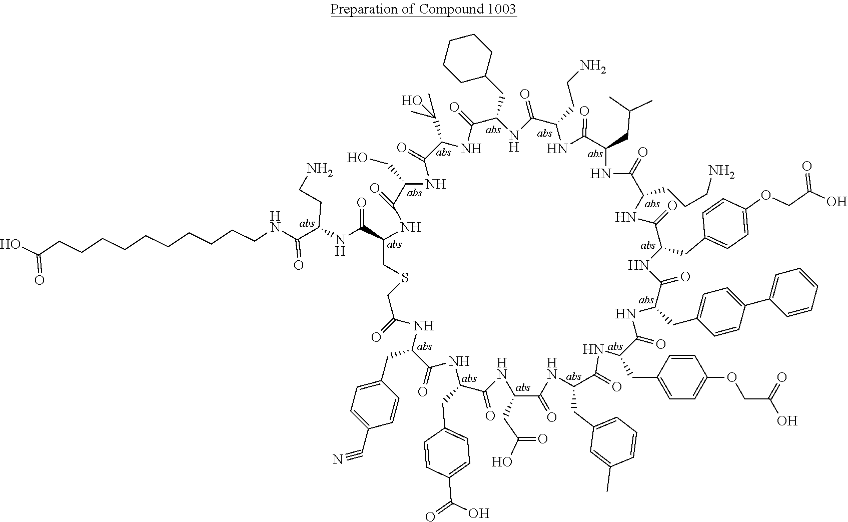 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(C#N)cc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

To a 45-mL polypropylene solid-phase reaction vessel was added 2-Chlorotrityl resin pre-loaded with 11-((((9H-fluoren-9-yl) methoxy) carbonyl)amino) undecanoic acid (180 mg, 0.100 mmol), and the reaction vessel was placed on the Prelude peptide synthesizer. The following procedures were then performed sequentially:

“Prelude Resin-swelling procedure” was followed; “Prelude Single-coupling procedure” was followed with Fmoc-Dab(Boc)-OH; “Prelude Single-coupling procedure” was followed with Fmoc-Cys(Trt)-OH; “Prelude Single-coupling procedure” was followed with Fmoc-Ser(tBu)-OH; “Prelude Single-coupling procedure” was followed with Fmoc-Val(β-OH)—OH; “Prelude Single-coupling procedure” was followed with Fmoc-Cha-OH; “Prelude Single-coupling procedure” was followed with Fmoc-Dab(Boc)-OH; “Prelude Single-coupling procedure” or “Prelude double-coupling procedure” was followed with Fmoc-D-Leu-OH; “Prelude Single-coupling procedure” was followed with Fmoc-Orn(Boc)-OH; “Prelude Single-coupling procedure” was followed with Fmoc-Tyr(CH₂COOtBu)-OH; “Prelude Single-coupling procedure” was followed with Fmoc-Bip-OH; “Prelude Single-coupling procedure” was followed with Fmoc-Tyr(CH₂COOtBu)-OH; “Prelude Single-coupling procedure” was followed with Fmoc-Phe (3-Me)-OH; “Prelude Single-coupling procedure” was followed with Fmoc-Asp(tBu)-OH; The resin was split into 0.025 mmol and was transferred to a different 45-mL polypropylene solid-phase reaction vessel, and the reaction vessel was placed on the Symphony X peptide synthesizer. The following procedures were then performed sequentially: “Symphony X Resin-swelling procedure” was followed; “Symphony X Single-coupling procedure” was followed with Phe (4-COOtBu)-OH; “Symphony X Single-Coupling Manual Addition Procedure A” was followed with Phe (4-CN); “Symphony X Chloroacetic Anhydride coupling procedure” was followed; “Symphony X Final rinse and dry procedure” was followed; “Global Deprotection Method A” was followed; “Cyclization Method A” was followed.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.5 mg, and its estimated purity by LCMS analysis was 95.7%. Analysis condition 2: Retention time=1.76 min; ESI-MS(+) m/z [M+2H]²⁺: 1217.2.

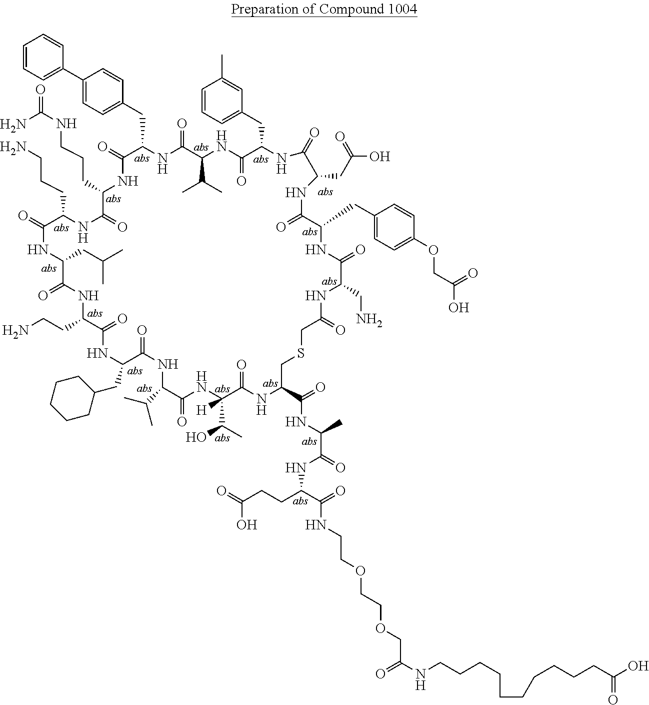 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1004 was prepared on a 50 μmol scale. The yield of the product was 15.5 mg, and its estimated purity by LCMS analysis was 98.4%. Analysis condition A: Retention time=1.71 min; ESI-MS(+) m/z [M+2H]²⁺: 812.

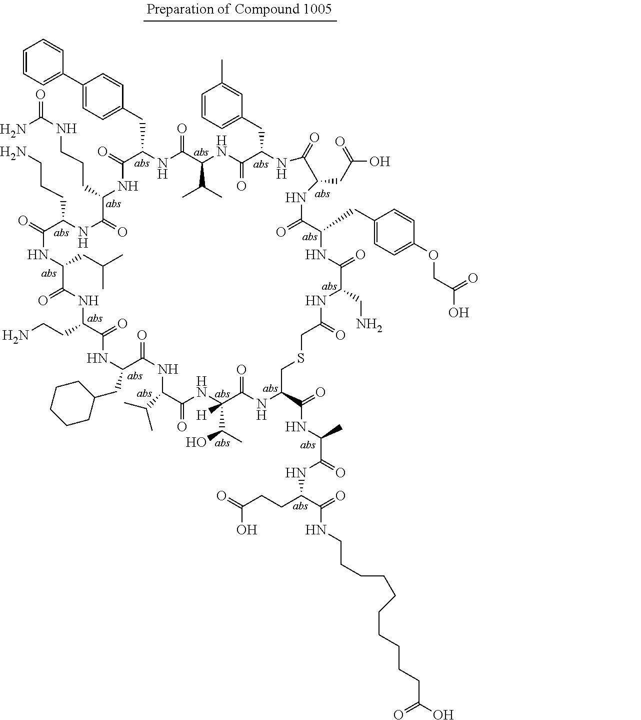 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1005 was prepared on a 50 μmol scale. The yield of the product was 16 mg, and its estimated purity by LCMS analysis was 94.2%. Analysis condition A: Retention time=1.51 min; ESI-MS(+) m/z [M+2H]²⁺: 1145.

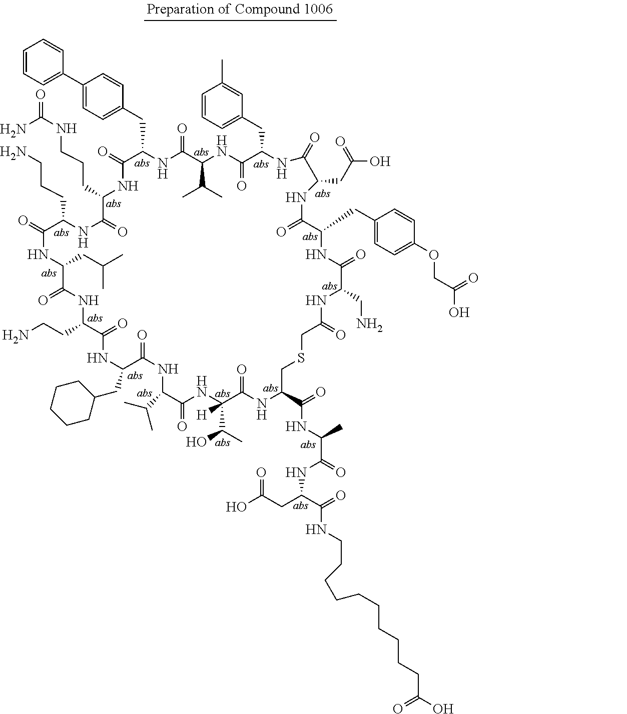 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1006 was prepared on a 50 μmol scale. The yield of the product was 19.6 mg, and its estimated purity by LCMS analysis was 96.3%. Analysis condition B: Retention time=1.65 min; ESI-MS(+) m/z [M+3H]³⁺: 759.

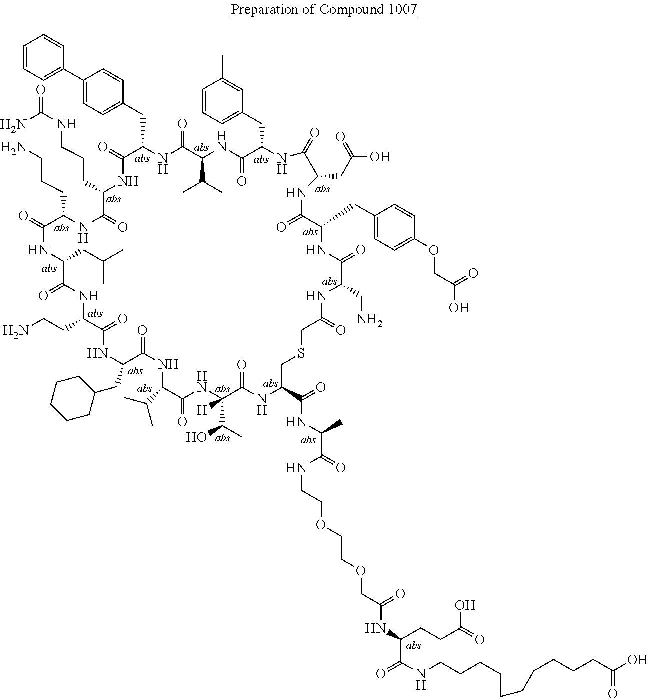 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)NCCOCCOCC(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1007 was prepared on a 50 μmol scale. The yield of the product was 36.3 mg, and its estimated purity by LCMS analysis was 93.5%. Analysis condition A: Retention time=1.48 min; ESI-MS(+) m/z [M+3H]³⁺: 812.1.

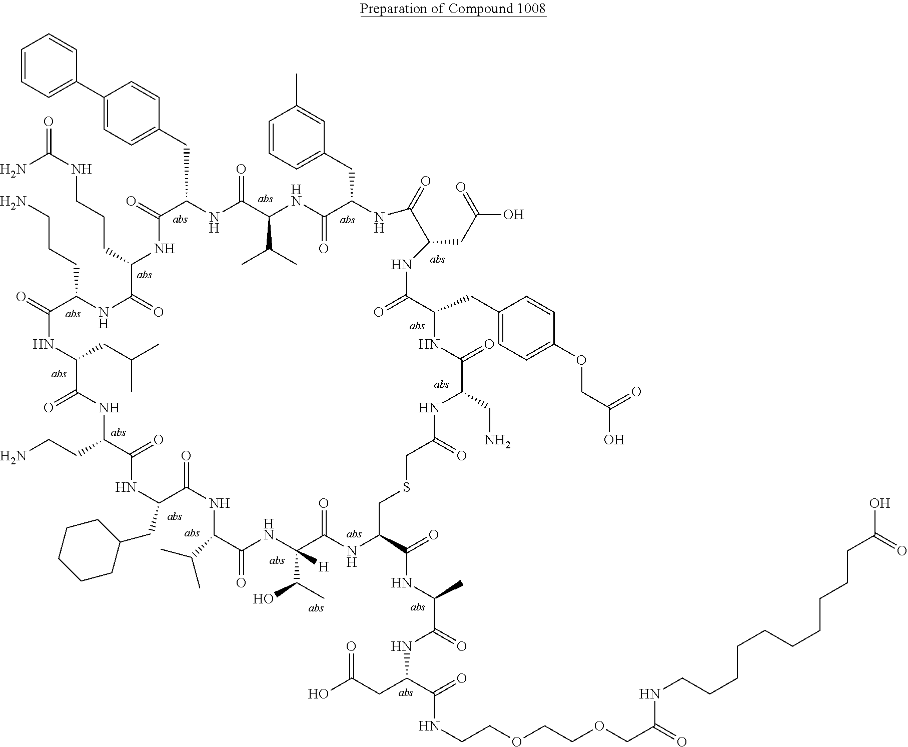 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)O)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1008 was prepared on a 50 μmol scale. The yield of the product was 14.1 mg, and its estimated purity by LCMS analysis was 92.2%. Analysis condition A: Retention time=1.51 min; ESI-MS(+) m/z [M+3H]³⁺: 807.2.

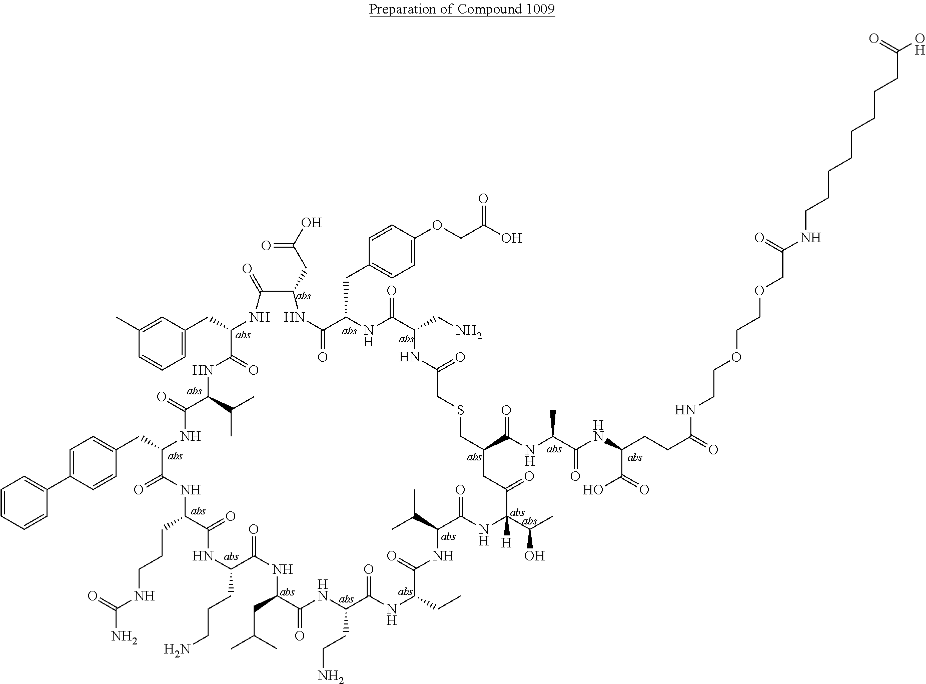 [SMILES: CC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCC(=O)NCCOCCOCC(=O)NCCCCCCCCC(=O)O)C(=O)O)CC(=O)[C@H]([C@@H](C)O)NC(=O)[C@H](C(C)C)NC1=O]

Compound 1009 was prepared on a 50 μmol scale. The yield of the product was 13.4 mg, and its estimated purity by LCMS analysis was 93.6%. Analysis condition A: Retention time=1.64 min; ESI-MS(+) m/z [M+3H]³⁺: 812.

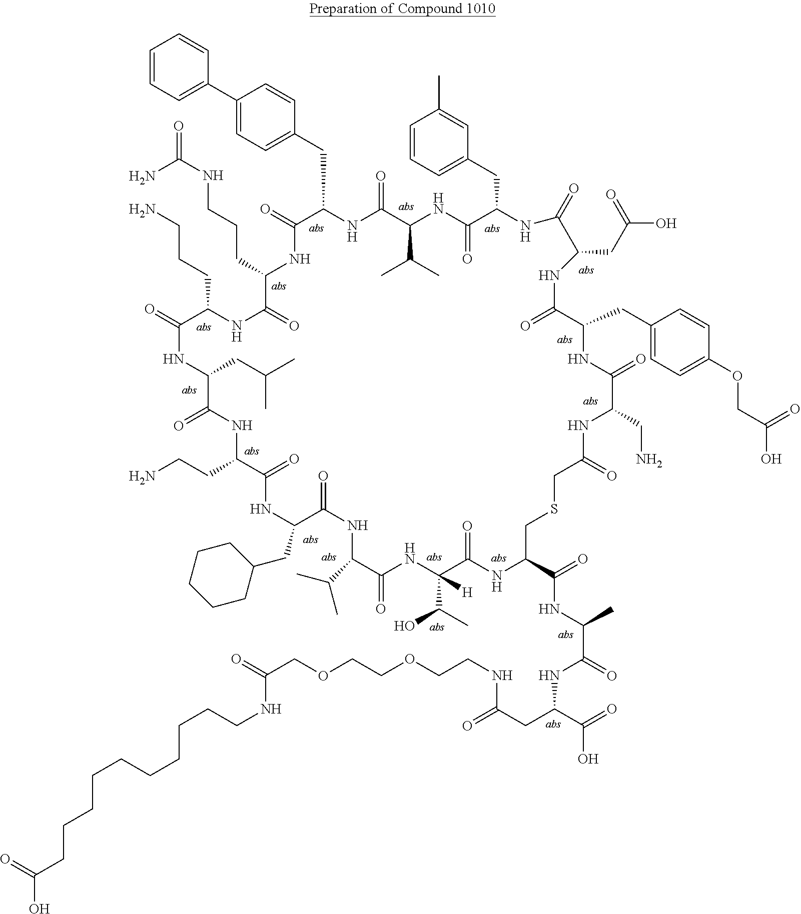 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1010 was prepared on a 50 μmol scale. The yield of the product was 4.5 mg, and its estimated purity by LCMS analysis was 95.2%. Analysis condition B: Retention time=1.57 min; ESI-MS(+) m/z [M+3H]³⁺: 807.2.

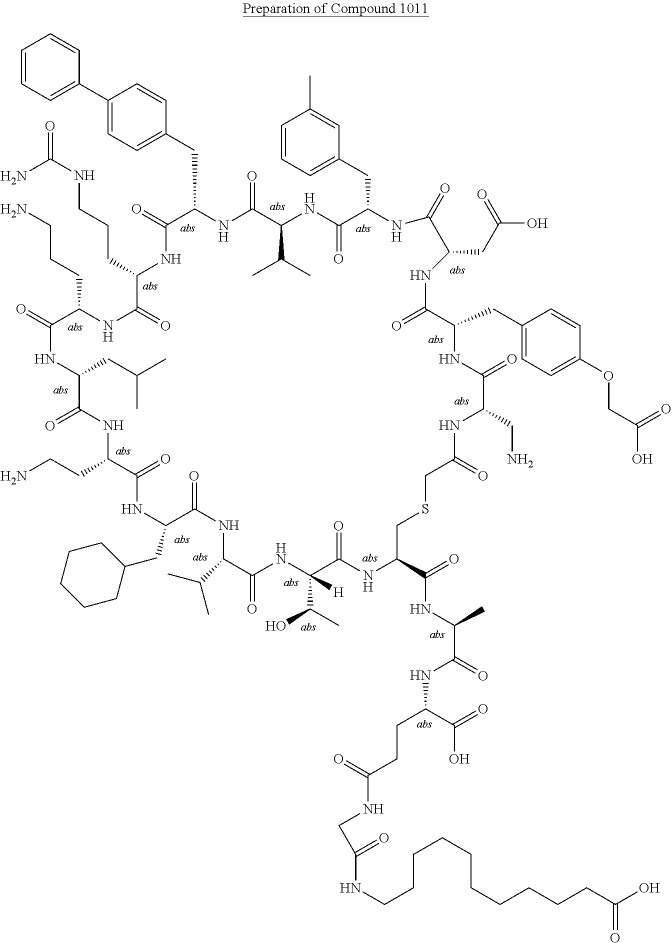 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)NCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1011 was prepared on a 50 μmol scale. The yield of the product was 13.3 mg, and its estimated purity by LCMS analysis was 90.3%. Analysis condition B: Retention time=1.51 min; ESI-MS(+) m/z [M+3H]³⁺: 782.4.

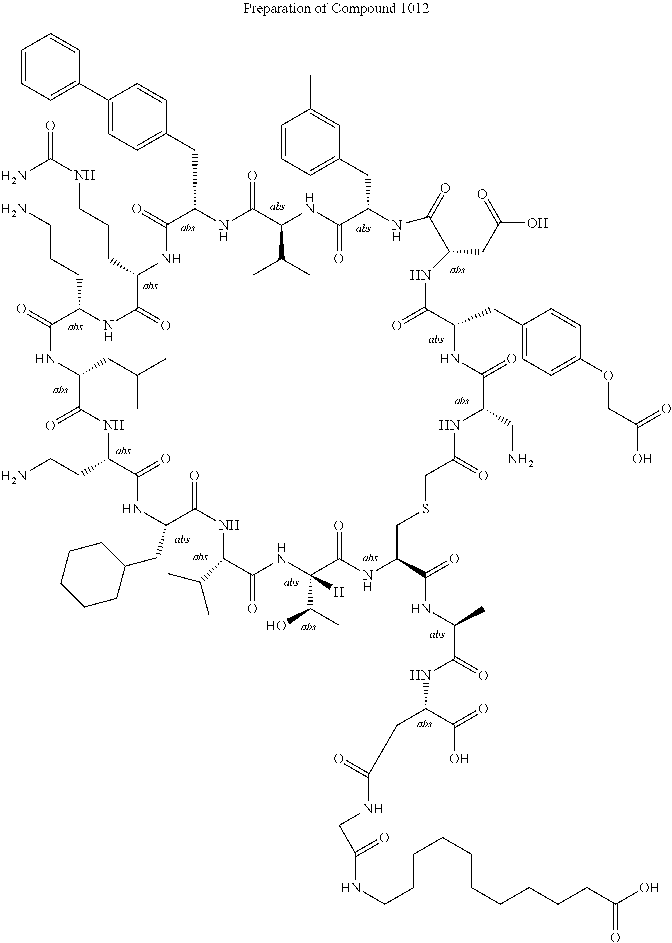 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)NCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1012 was prepared on a 50 μmol scale. The yield of the product was 12.7 mg, and its estimated purity by LCMS analysis was 90.6%. Analysis condition B: Retention time=1.48 min; ESI-MS(+) m/z [M+3H]³⁺: 777.9.

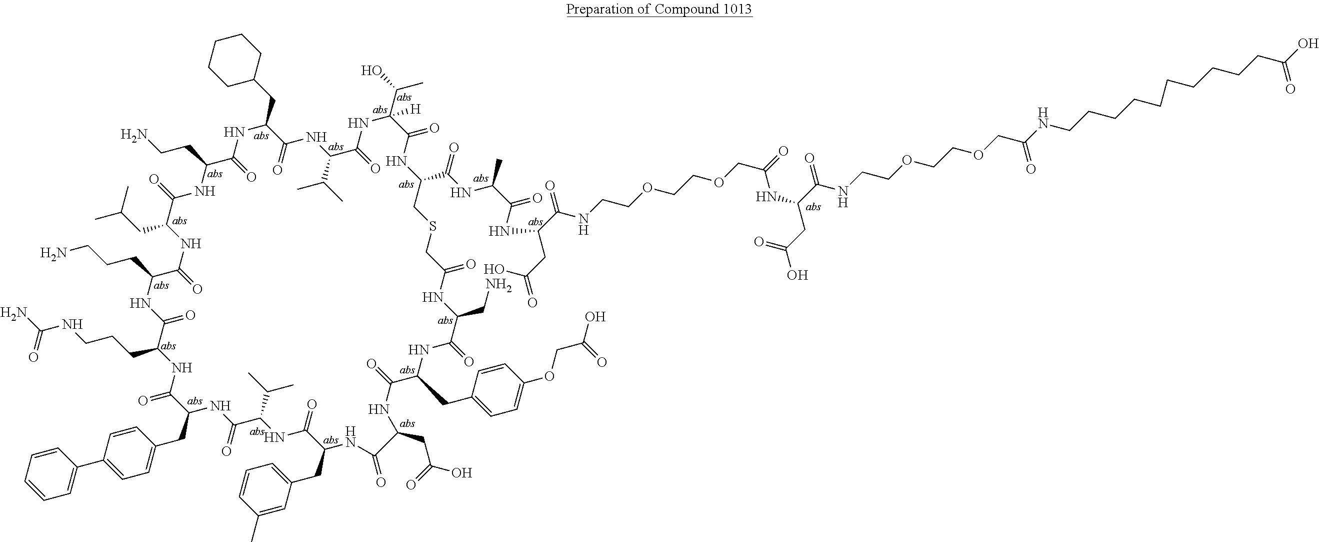 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)O)C(=O)NCCOCCOCC(=O)N[C@@H](CC(=O)O)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1013 was prepared on a 50 μmol scale. The yield of the product was 4.3 mg, and its estimated purity by LCMS analysis was 90.3%. Analysis condition B: Retention time=1.64 min; ESI-MS(+) m/z [M+3H]³⁺: 894.1.

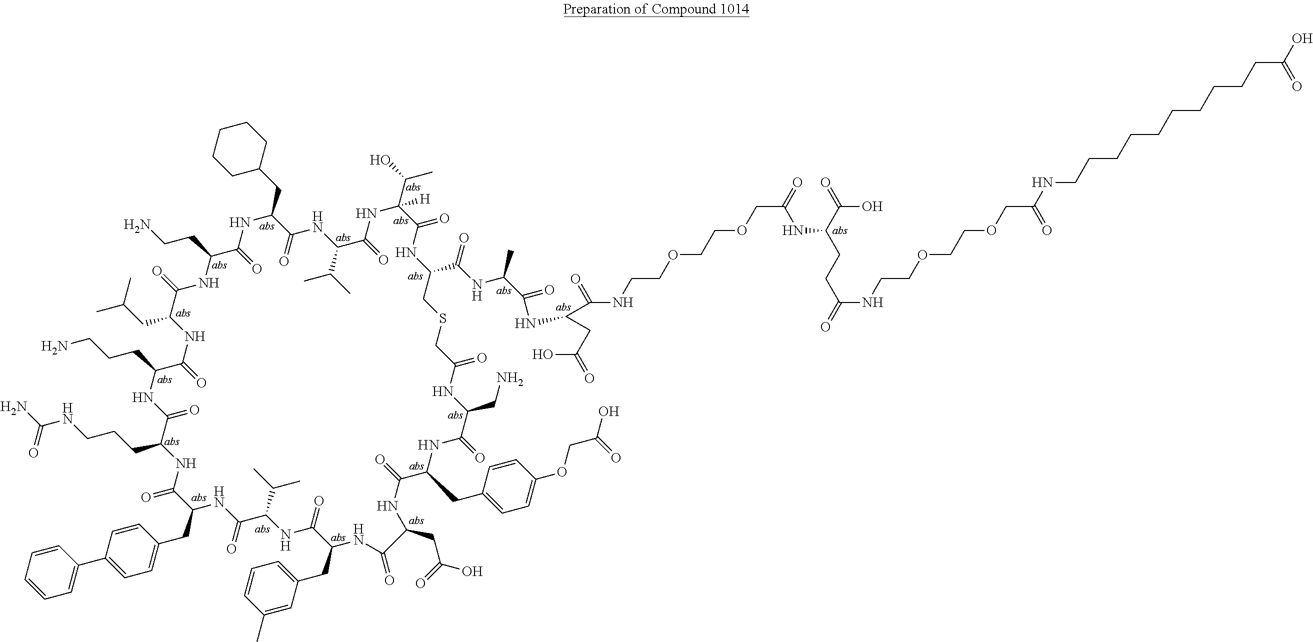 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)O)C(=O)NCCOCCOCC(=O)N[C@@H](CCC(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1014 was prepared on a 50 μmol scale. The yield of the product was 9.3 mg, and its estimated purity by LCMS analysis was 97.3%. Analysis condition B: Retention time=1.63 min; ESI-MS(+) m/z [M+3H]³⁺: 899.2.

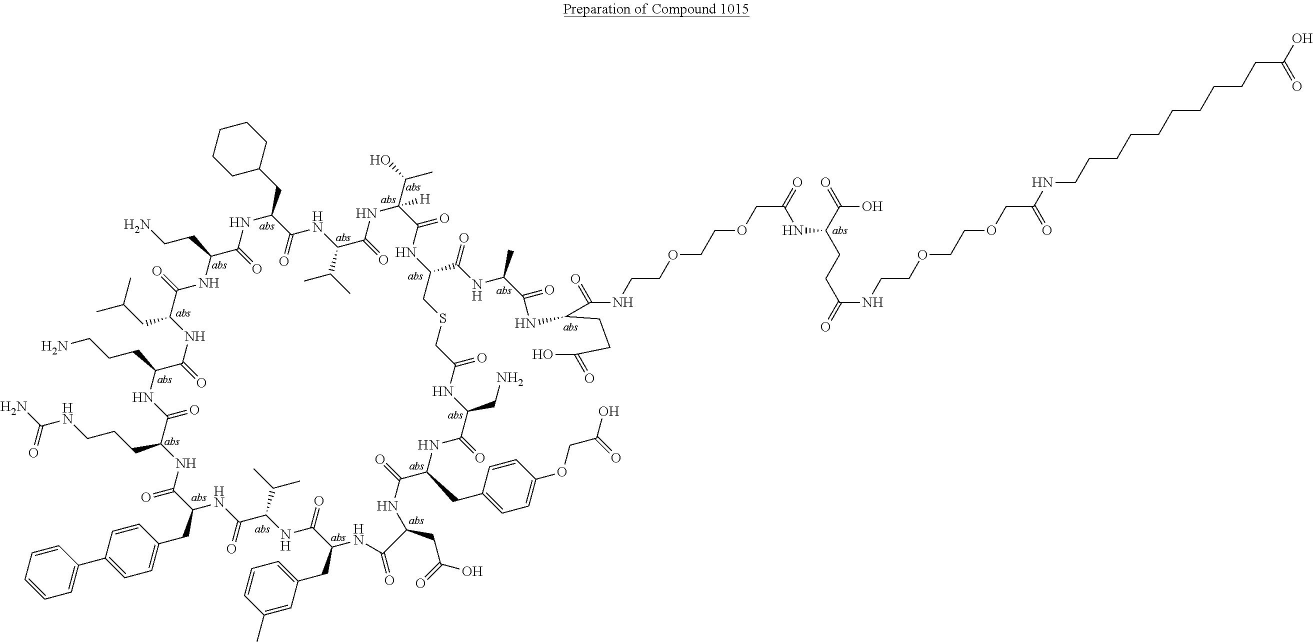 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCOCCOCC(=O)N[C@@H](CCC(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1015 was prepared on a 50 μmol scale. The yield of the product was 10.5 mg, and its estimated purity by LCMS analysis was 90.6%. Analysis condition A: Retention time=1.43 min; ESI-MS(+) m/z [M+2H]²⁺: 1354.9.

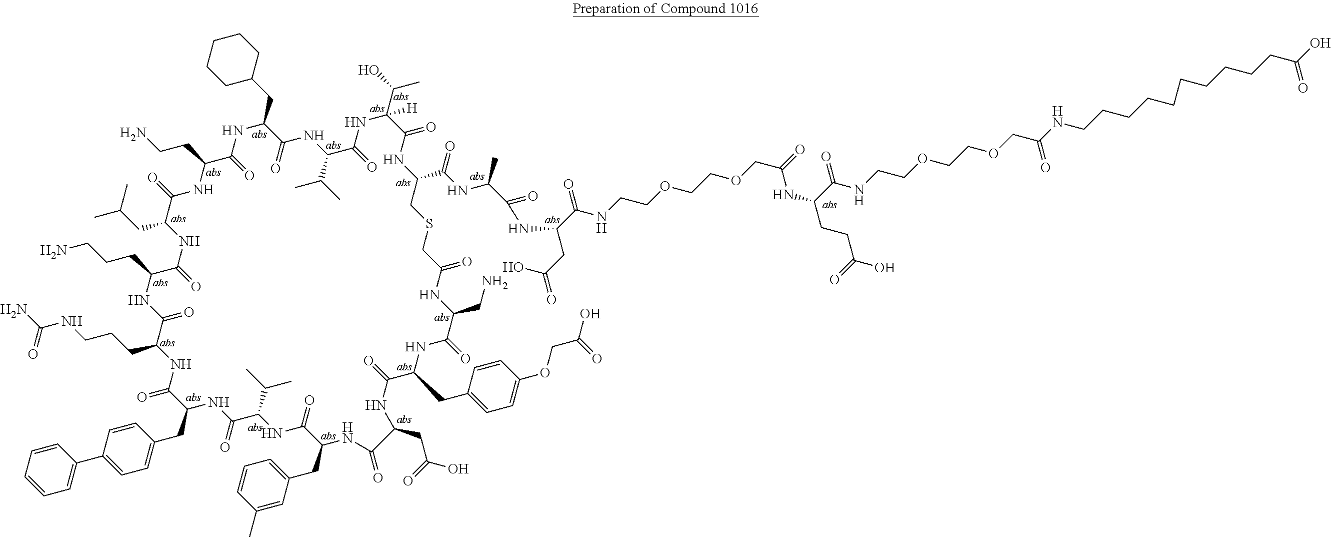 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)O)C(=O)NCCOCCOCC(=O)N[C@@H](CCC(=O)O)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1016 was prepared on a 50 μmol scale. The yield of the product was 7.6 mg, and its estimated purity by LCMS analysis was 91%. Analysis condition B: Retention time=1.63 min; ESI-MS(+) m/z [M+3H]³⁺: 899.2.

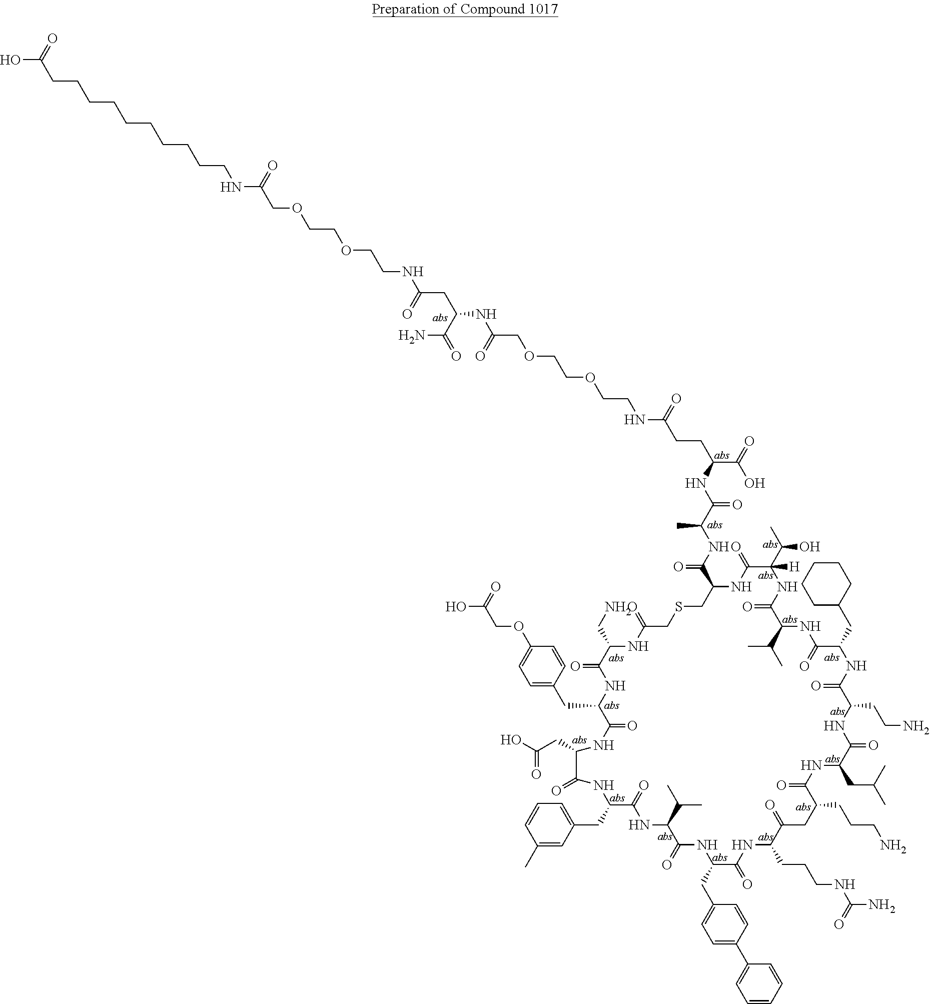 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)NCCOCCOCC(=O)N[C@@H](CC(=O)NCCOCCOCC(=O)NCCCCCCCCCCC(=O)O)C(N)=O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)CC(=O)[C@H](CCCNC(N)=O)NC(=O)[C@H](Cc3ccc(-c4ccccc4)cc3)NC(=O)[C@H](C(C)C)NC2=O)c1]

Compound 1017 was prepared on a 50 μmol scale. The yield of the product was 17.1 mg, and its estimated purity by LCMS analysis was 90.7%. Analysis condition A: Retention time=1.45 min; ESI-MS(+) m/z [M+3H]³⁺: 899.

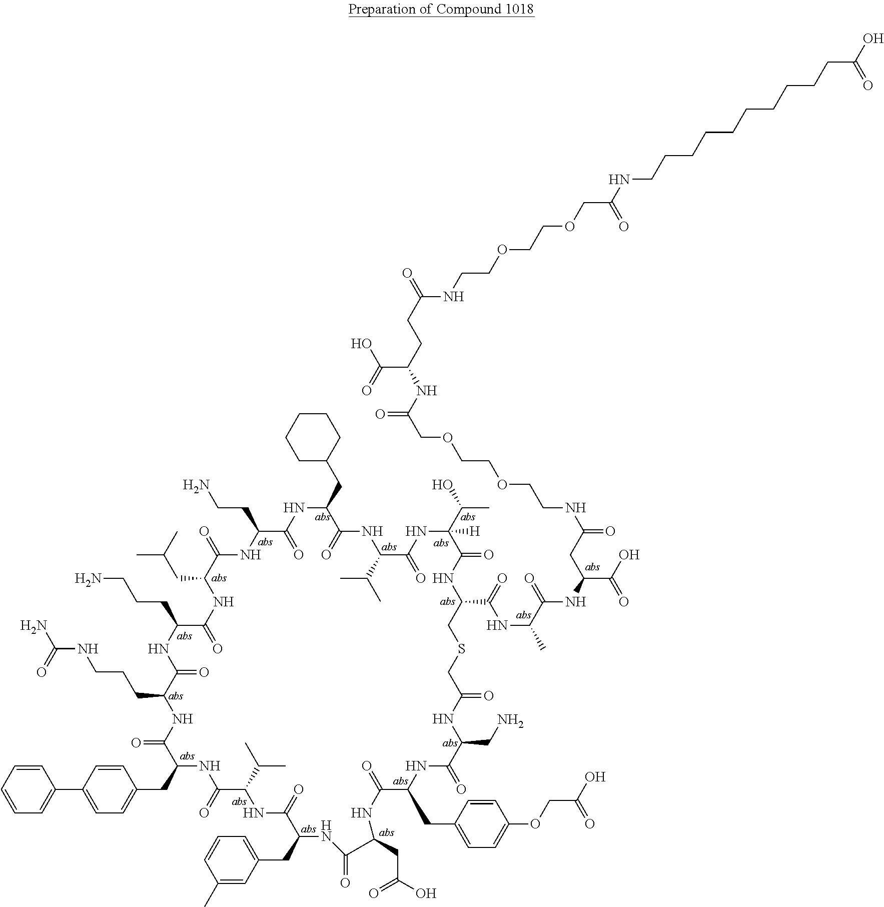 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)NCCOCCOCC(=O)N[C@@H](CCC(=O)NCCOCCOCC(=O)NCCCCCCCCCCC(=O)O)C(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1018 was prepared on a 50 μmol scale. The yield of the product was 8.5 mg, and its estimated purity by LCMS analysis was 91.5%. Analysis condition B: Retention time=1.64 min; ESI-MS(+) m/z [M+3H]³⁺:899.1.

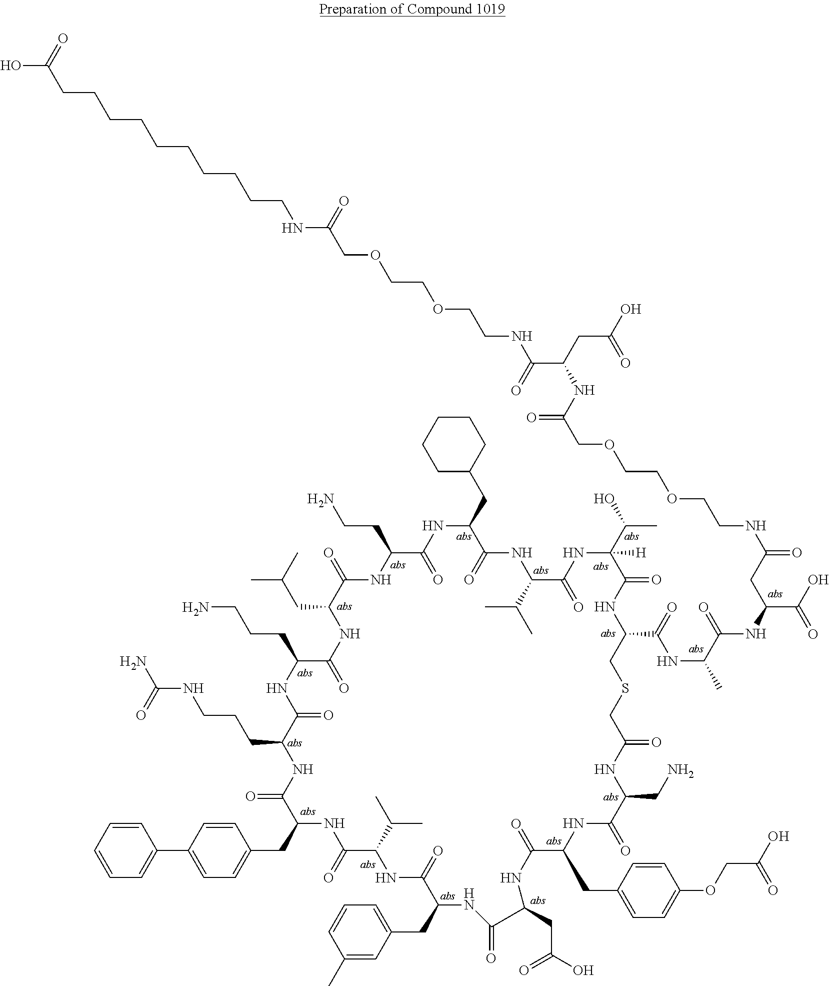 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)NCCOCCOCC(=O)N[C@@H](CC(=O)O)C(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1019 was prepared on a 50 μmol scale. The yield of the product was 12.4 mg, and its estimated purity by LCMS analysis was 92.3%. Analysis condition B: Retention time=1.48 min; ESI-MS(+) m/z [M+3H]³⁺: 894.1.

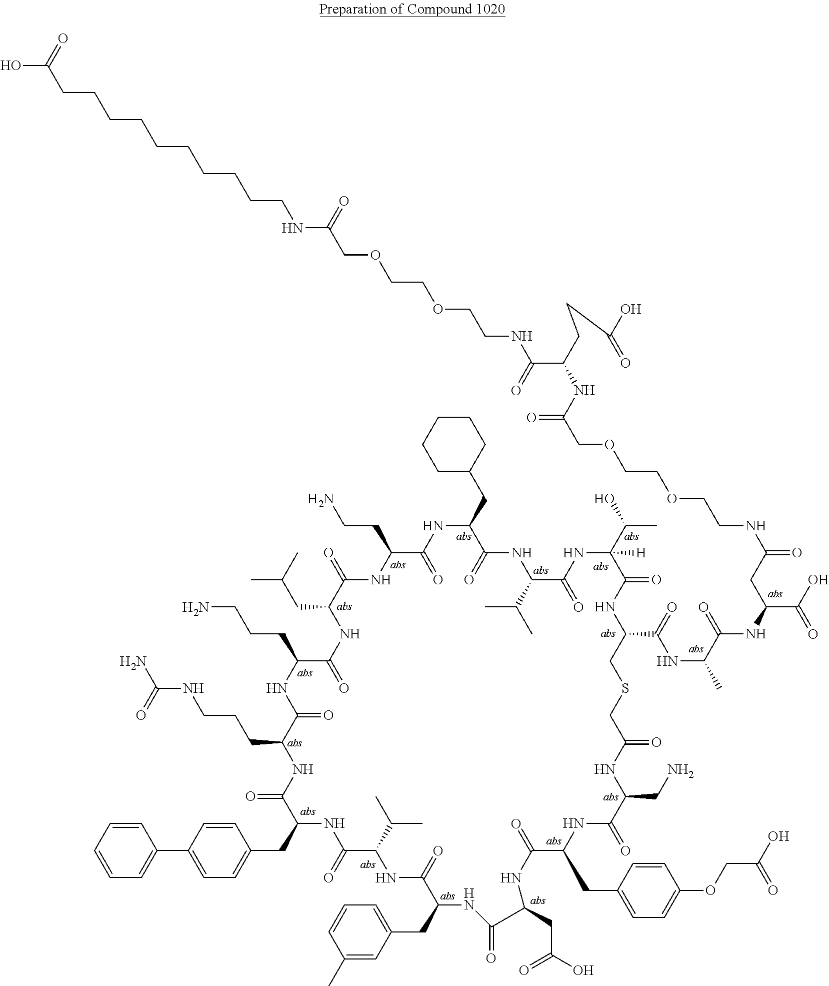 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)NCCOCCOCC(=O)N[C@@H](CCC(=O)O)C(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1020 was prepared on a 50 μmol scale. The yield of the product was 8.2 mg, and its estimated purity by LCMS analysis was 91.6%. Analysis condition A: Retention time=1.58 min; ESI-MS(+) m/z [M+3H]³⁺: 899.1.

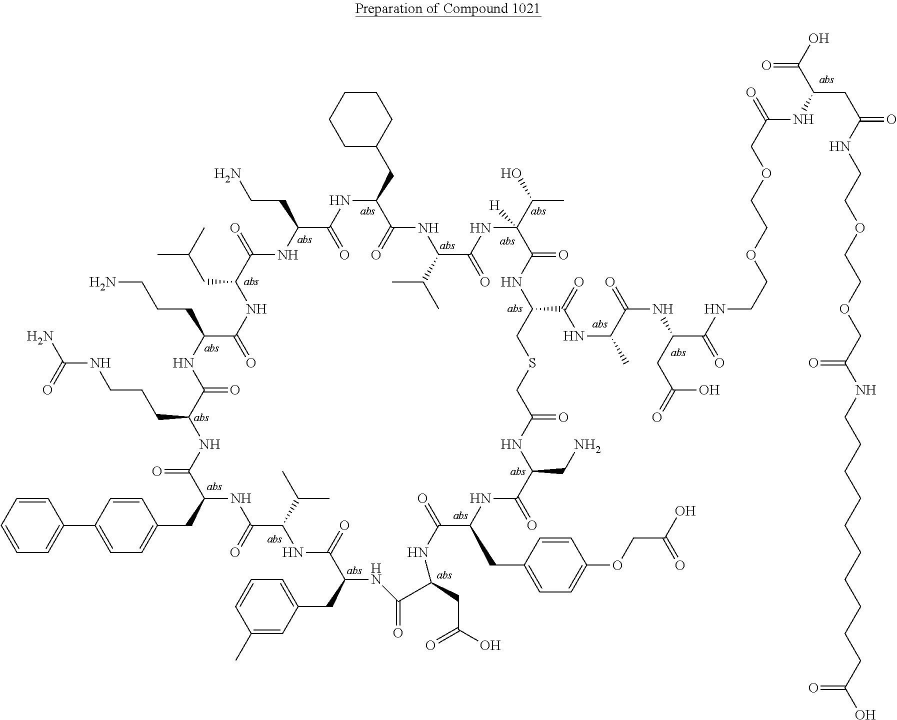 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)O)C(=O)NCCOCCOCC(=O)N[C@@H](CC(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1021 was prepared on a 50 μmol scale. The yield of the product was 10.9 mg, and its estimated purity by LCMS analysis was 100%. Analysis condition A: Retention time=1.54 min; ESI-MS(+) m/z [M+3H]³⁺: 894.1.

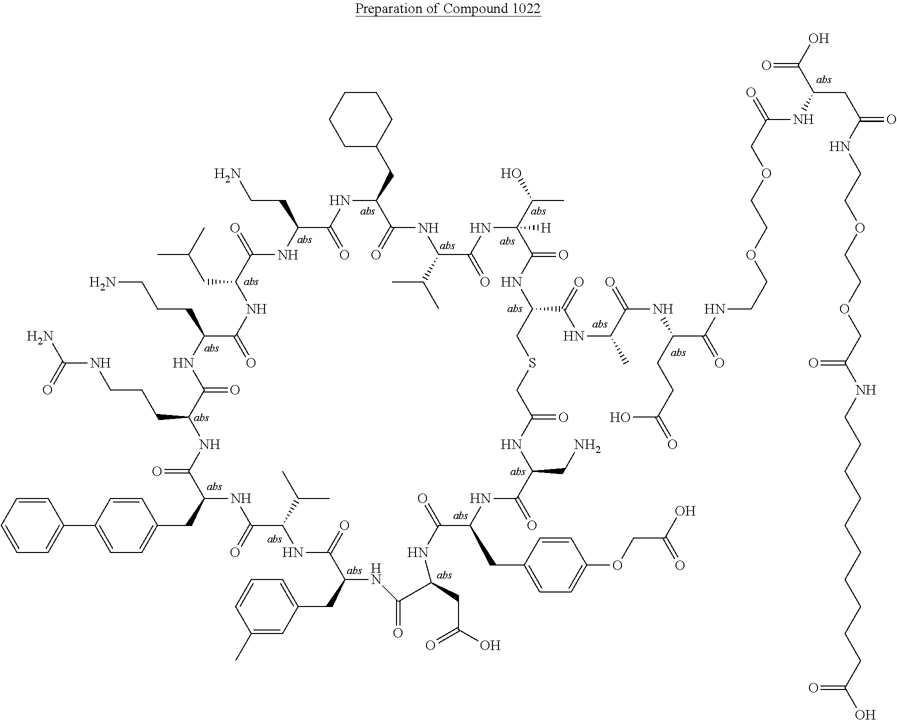 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCOCCOCC(=O)N[C@@H](CC(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1022 was prepared on a 50 μmol scale. The yield of the product was 4.7 mg, and its estimated purity by LCMS analysis was 90.4%. Analysis condition A: Retention time=1.59 min; ESI-MS(+) m/z [M+3H]³⁺: 899.1.

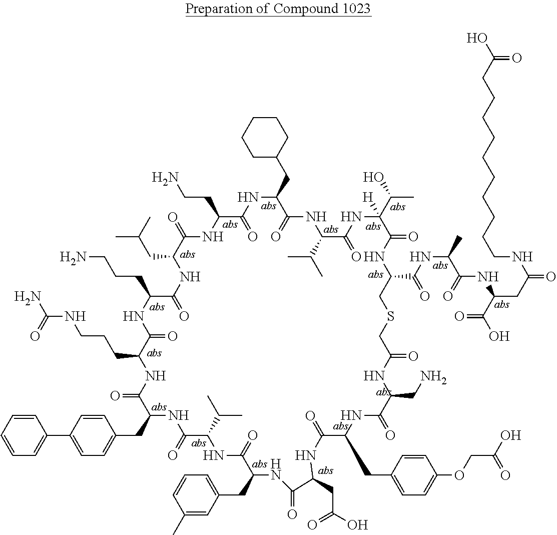 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1023 was prepared on a 50 μmol scale. The yield of the product was 14 mg, and its estimated purity by LCMS analysis was 94.2%. Analysis condition A: Retention time=1.64 min; ESI-MS(+) m/z [M+2H]²⁺: 1138.3.

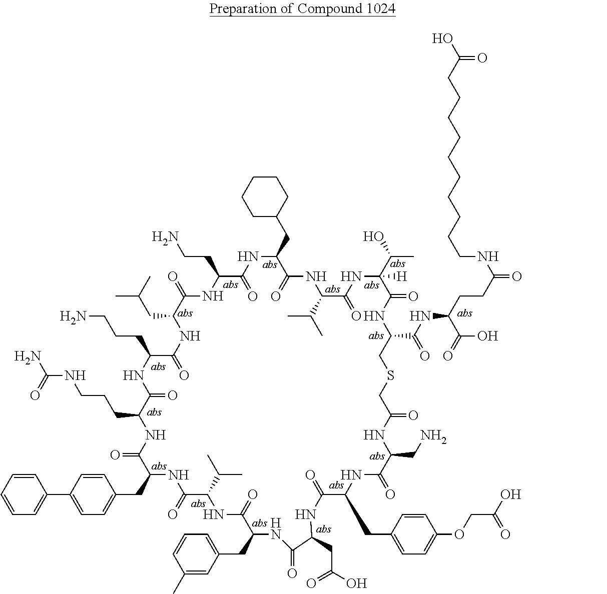 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1024 was prepared on a 50 μmol scale. The yield of the product was 11.8 mg, and its estimated purity by LCMS analysis was 92%. Analysis condition B: Retention time=1.72 min; ESI-MS(+) m/z [M+3H]³⁺: 740.

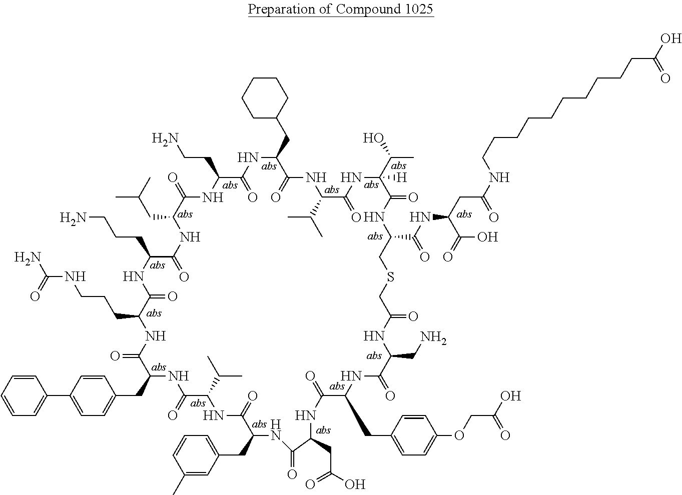 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1025 was prepared on a 50 μmol scale. The yield of the product was 17.7 mg, and its estimated purity by LCMS analysis was 91.4%. Analysis condition A: Retention time=1.56 min; ESI-MS(+) m/z [M+2H]²⁺: 1102.8.

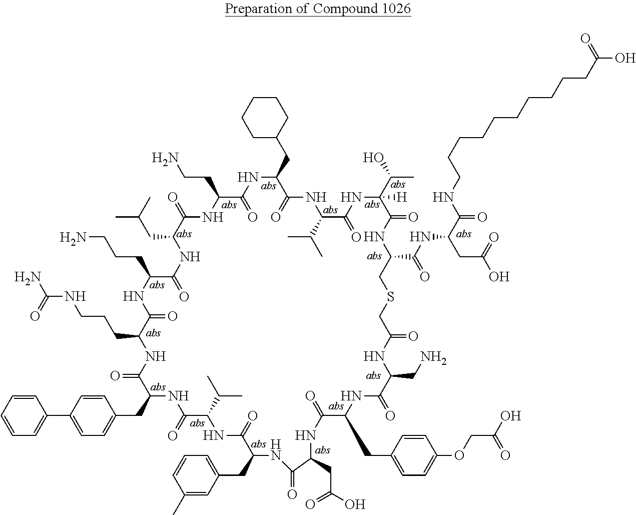 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1026 was prepared on a 50 μmol scale. The yield of the product was 22.7 mg, and its estimated purity by LCMS analysis was 90.5%. Analysis condition B: Retention time=1.66 min; ESI-MS(+) m/z [M+3H]³⁺: 735.3.

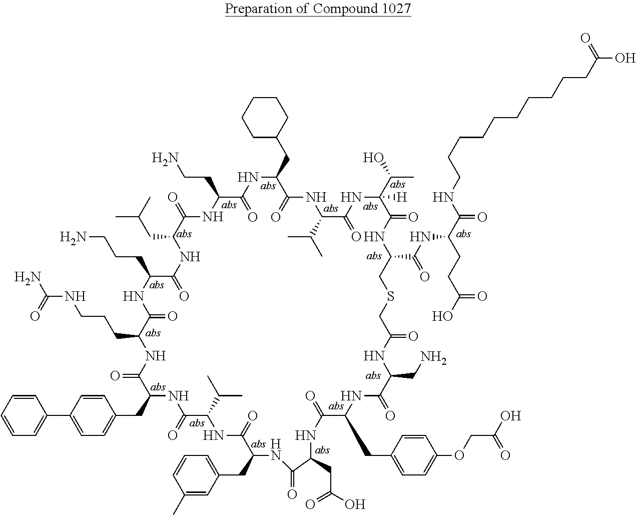 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1027 was prepared on a 50 μmol scale. The yield of the product was 8.9 mg, and its estimated purity by LCMS analysis was 90.1%. Analysis condition A: Retention time=1.44 min; ESI-MS(+) m/z [M+2H]²⁺: 1109.2.

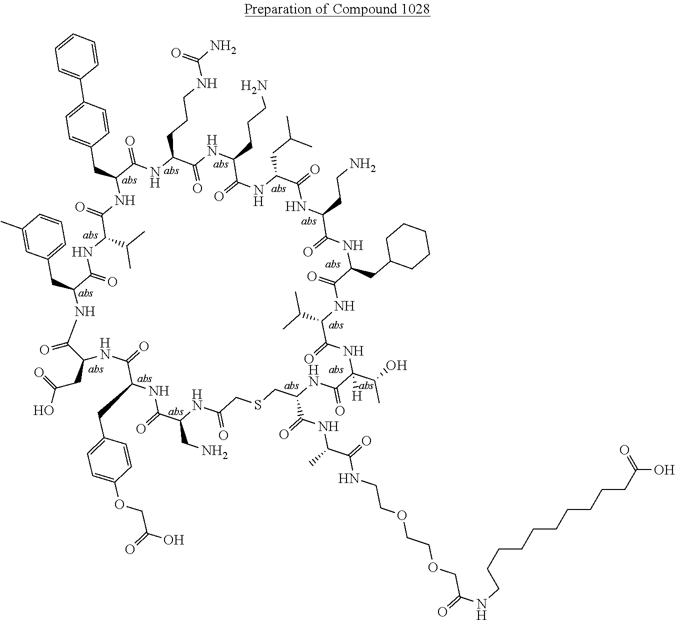 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1028 was prepared on a 50 μmol scale. The yield of the product was 23.4 mg, and its estimated purity by LCMS analysis was 94.6%. Analysis condition B: Retention time=1.66 min; ESI-MS(+) m/z [M+2H]²⁺: 1146.5.

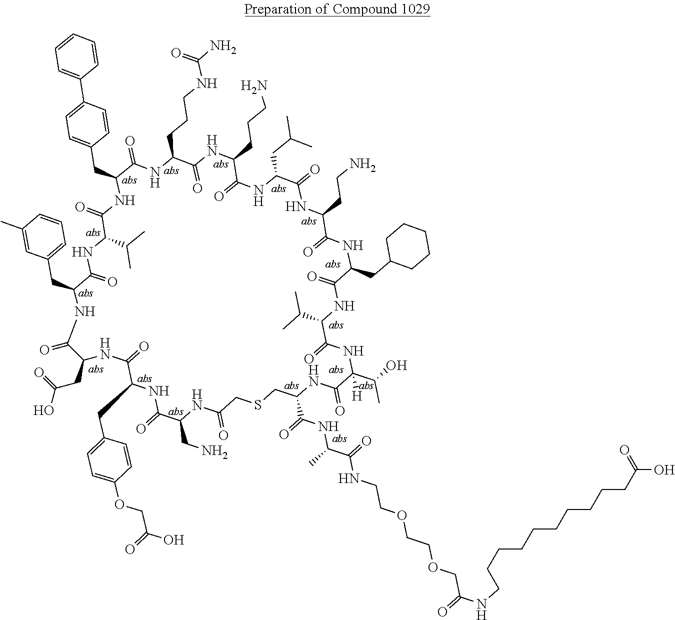 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1029 was prepared on a 50 μmol scale. The yield of the product was 8.3 mg, and its estimated purity by LCMS analysis was 95.1%. Analysis condition B: Retention time=1.67 min; ESI-MS(+) m/z [M+3H]³⁺: 769.2.

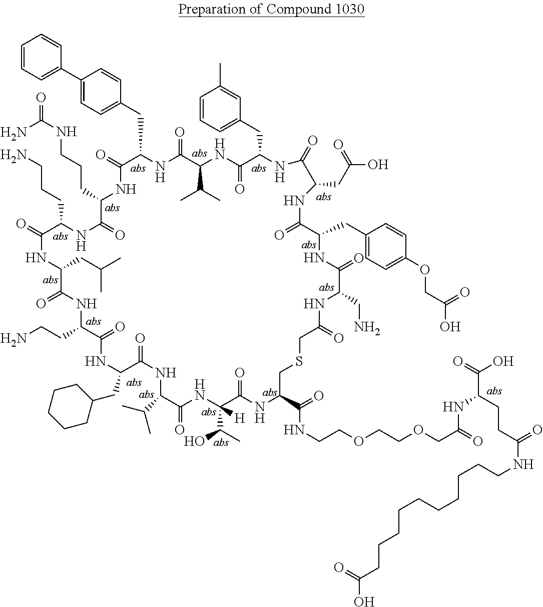 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1030 was prepared on a 50 μmol scale. The yield of the product was 14.3 mg, and its estimated purity by LCMS analysis was 93.4%. Analysis condition A: Retention time=1.65, 1.71 min; ESI-MS(+) m/z [M+2H]²⁺: 1182.12, 1182.1.

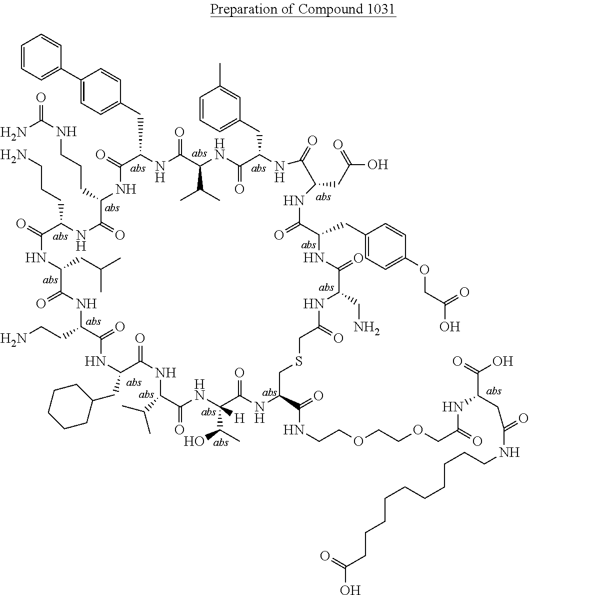 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1031 was prepared on a 50 μmol scale. The yield of the product was 23.3 mg, and its estimated purity by LCMS analysis was 92.3%. Analysis condition A: Retention time=1.46 min; ESI-MS(+) m/z [M+2H]²⁺: 1175.1.

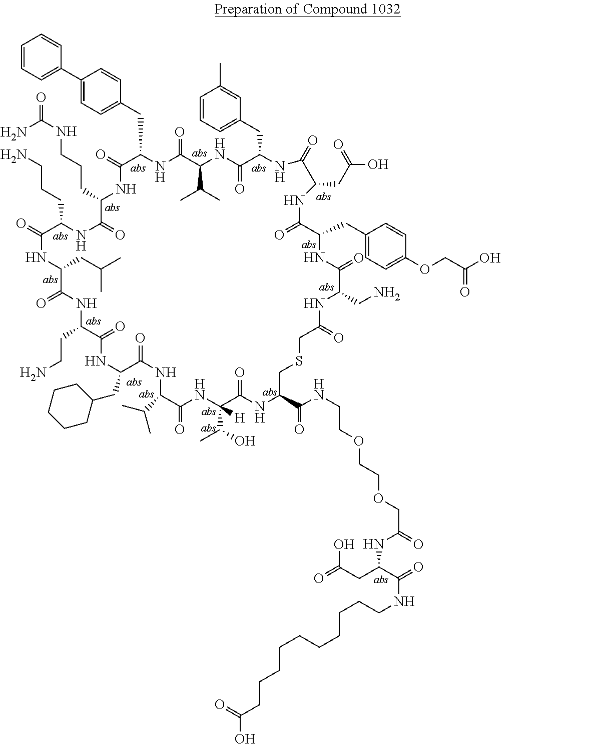 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1032 was prepared on a 50 μmol scale. The yield of the product was 25.9 mg, and its estimated purity by LCMS analysis was 93%. Analysis condition B: Retention time=1.7 min; ESI-MS(+) m/z [M+2H]²⁺: 1175.3.

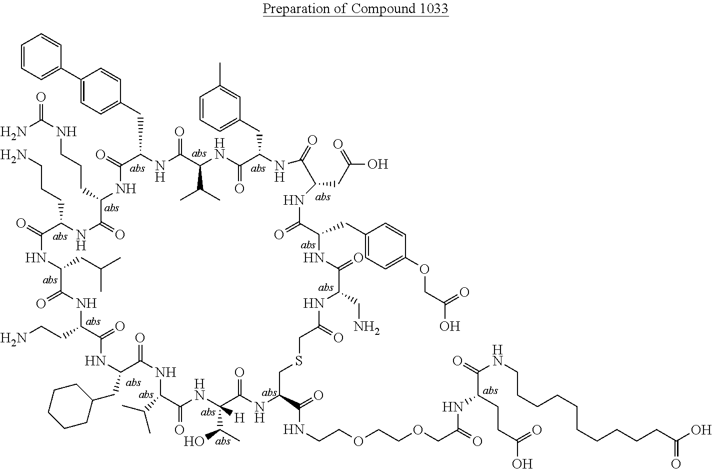 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1033 was prepared on a 50 μmol scale. The yield of the product was 15 mg, and its estimated purity by LCMS analysis was 96.7%. Analysis condition A: Retention time=1.62 min; ESI-MS(+) m/z [M+2H]²⁺: 1181.8.

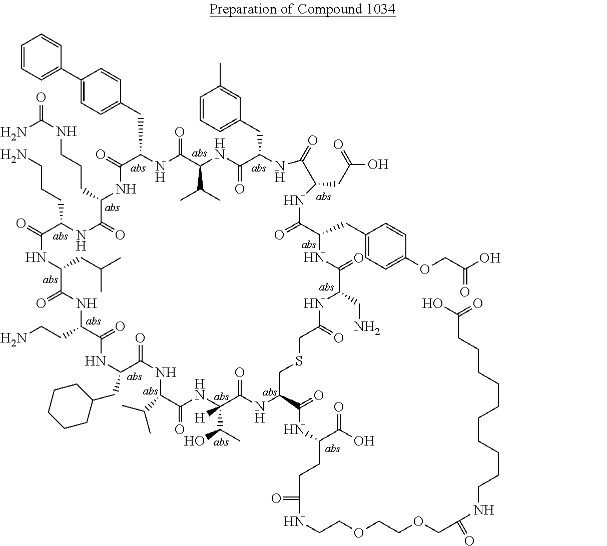 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCC(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1034 was prepared on a 50 μmol scale. The yield of the product was 11.2 mg, and its estimated purity by LCMS analysis was 100%. Analysis condition A: Retention time=1.71 min; ESI-MS(+) m/z [M+2H]²⁺: 1181.8.

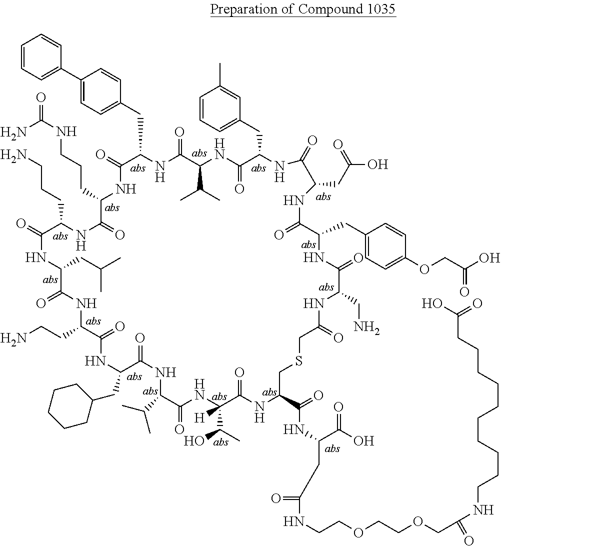 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)NCCOCCOCC(=O)NCCCCCCCCCCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1035 was prepared on a 50 μmol scale. The yield of the product was 25.6 mg, and its estimated purity by LCMS analysis was 83.1%. Analysis condition A: Retention time=1.5 min; ESI-MS(+) m/z [M+3H]³⁺: 784.

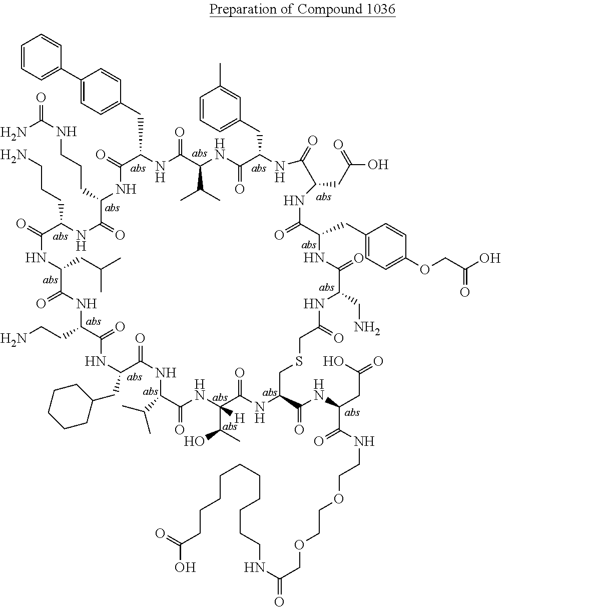 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)O)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1036 was prepared on a 50 μmol scale. The yield of the product was 10.9 mg, and its estimated purity by LCMS analysis was 100%. Analysis condition A: Retention time=1.71 min; ESI-MS(+) m/z [M+2H]²⁺: 1174.8.

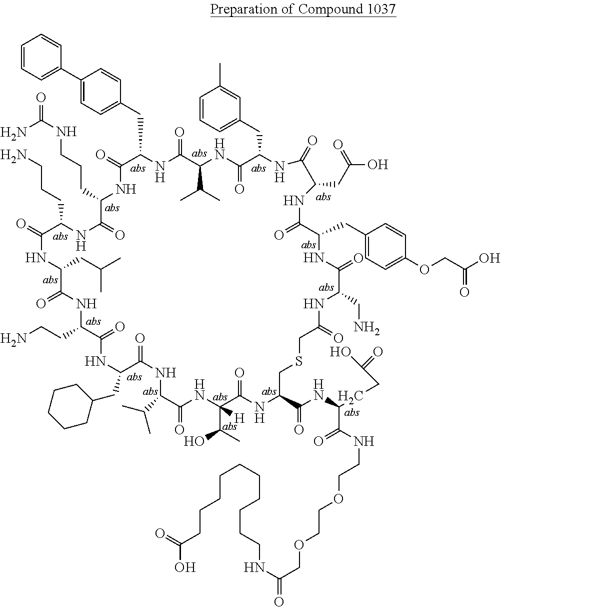 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCC(=O)O)C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1037 was prepared on a 50 μmol scale. The yield of the product was 9.8 mg, and its estimated purity by LCMS analysis was 94.7%. Analysis condition A: Retention time=1.65 min; ESI-MS(+) m/z [M+2H]²⁺: 1181.8.

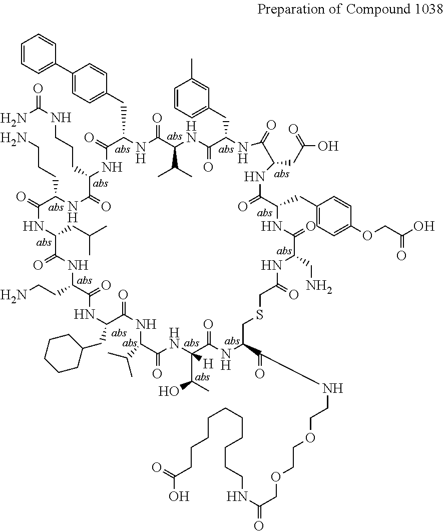 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1038 was prepared on a 50 μmol scale. The yield of the product was 12.1 mg, and its estimated purity by LCMS analysis was 98.8%. Analysis condition A: Retention time=1.74 min; ESI-MS(+) m/z [M+2H]²⁺: 1118.

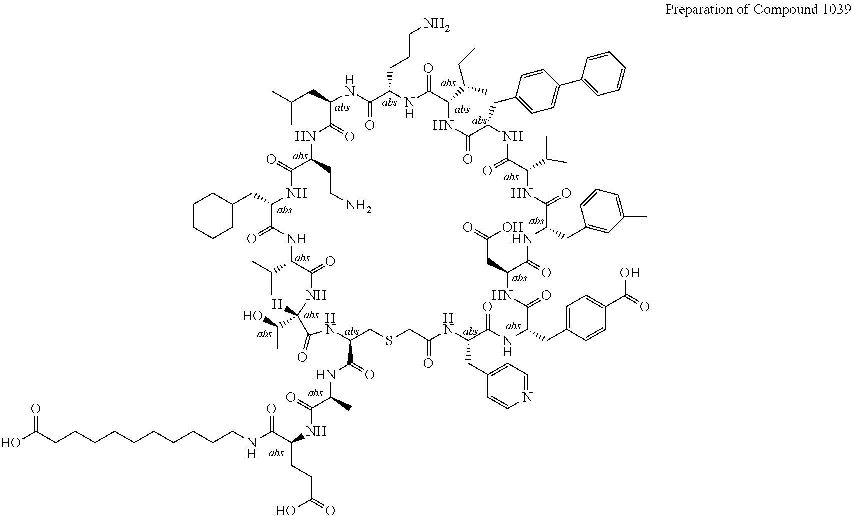 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1039 was prepared on a 100 μmol scale. The yield of the product was 11.7 mg, and its estimated purity by LCMS analysis was 93.1%. Analysis condition B: Retention time=1.61 min; ESI-MS(+) m/z [M+3H]³⁺: 760.

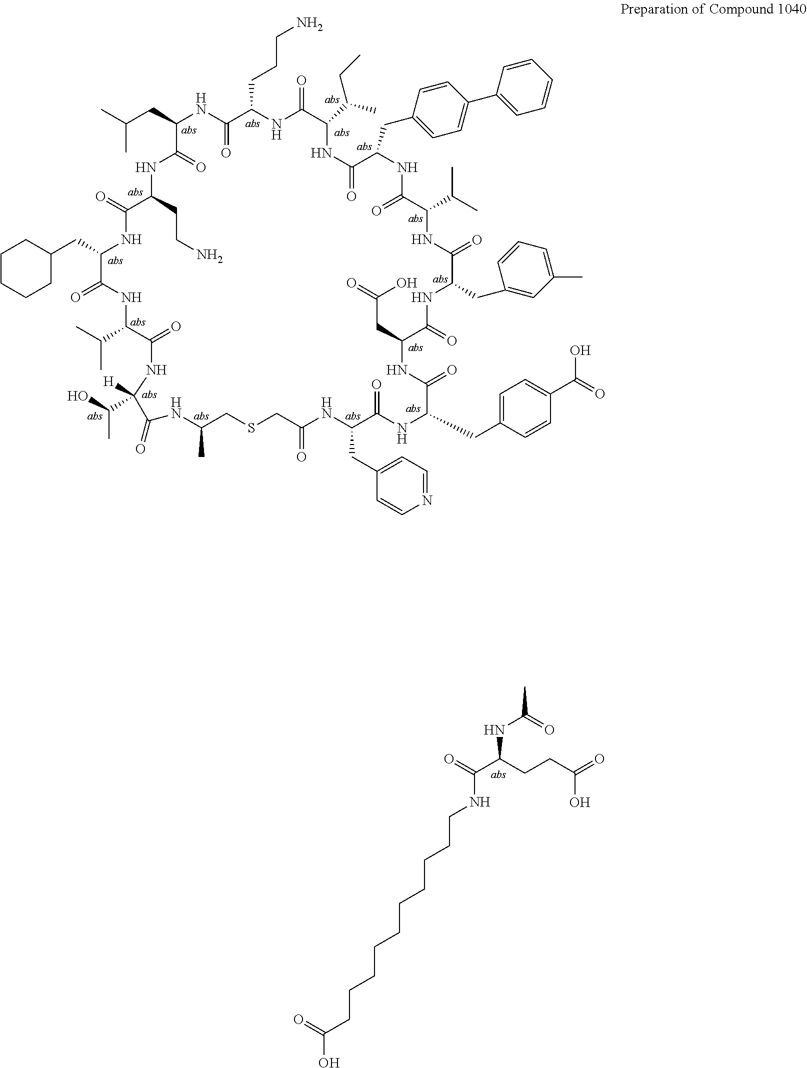

Compound 1040 was prepared on a 100 μmol scale. The yield of the product was 13.9 mg, and its estimated purity by LCMS analysis was 90.7%. Analysis condition B: Retention time=1.61 min; ESI-MS(+) m/z [M+3H]³⁺: 736.1.

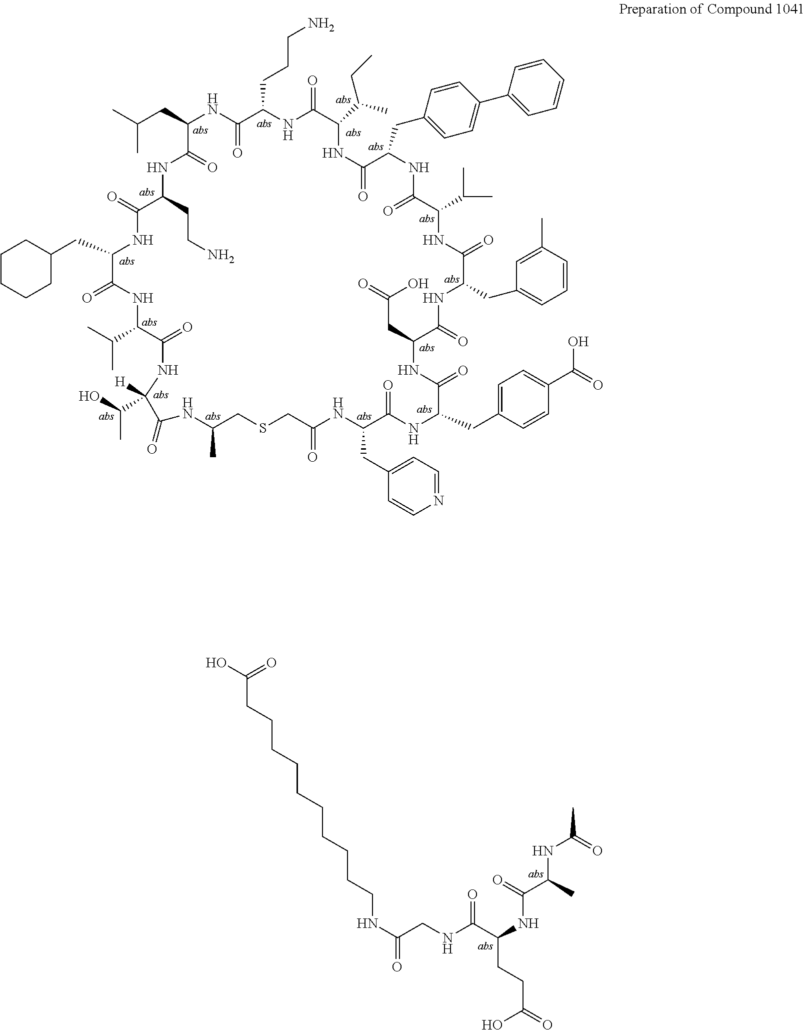

Compound 1041 was prepared on a 100 μmol scale. The yield of the product was 8.3 mg, and its estimated purity by LCMS analysis was 95.8%. Analysis condition B: Retention time=1.62 min; ESI-MS(+) m/z [M+3H]³⁺: 779.1.

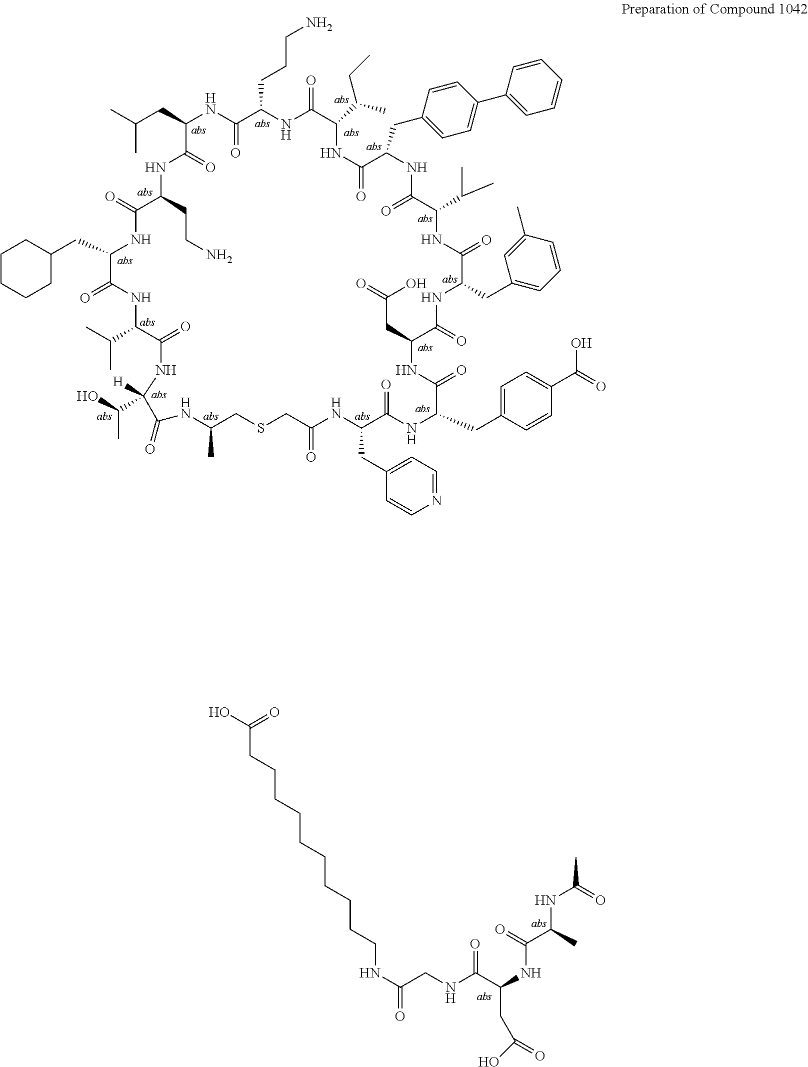

Compound 1042 was prepared on a 100 μmol scale. The yield of the product was 29.2 mg, and its estimated purity by LCMS analysis was 90.5%. Analysis condition B: Retention time=1.74 min; ESI-MS(+) m/z [M+3H]³⁺: 774.

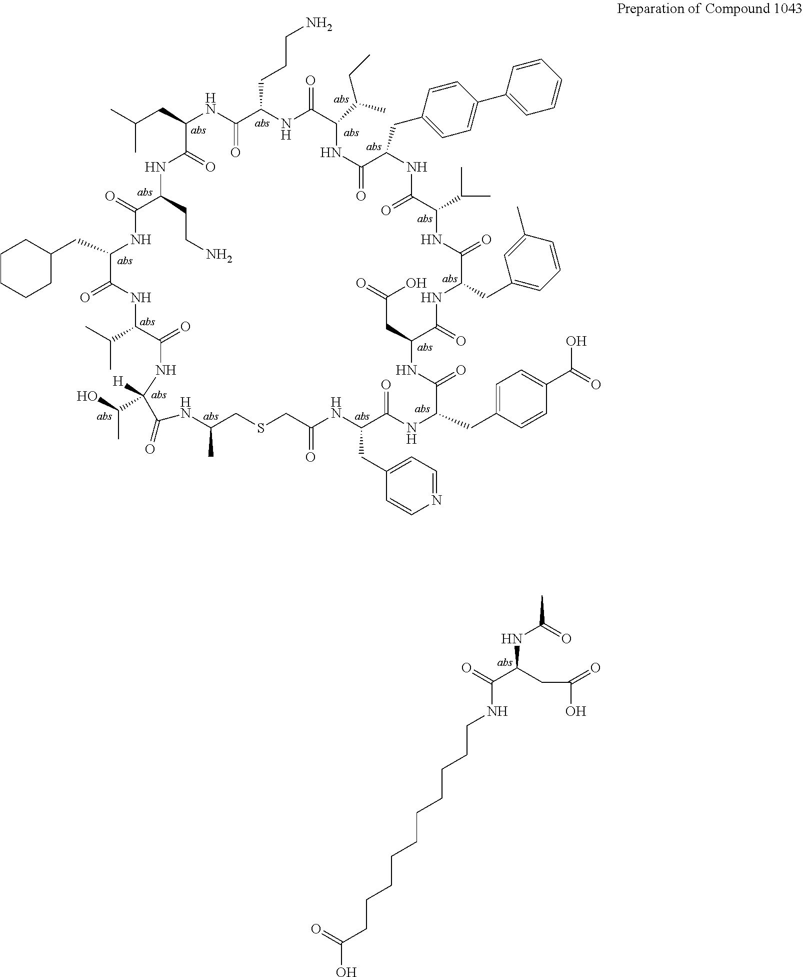

Compound 1043 was prepared on a 100 μmol scale. The yield of the product was 22.8 mg, and its estimated purity by LCMS analysis was 96.5%. Analysis condition B: Retention time=1.61 min; ESI-MS(+) m/z [M+3H]³⁺: 731.6.

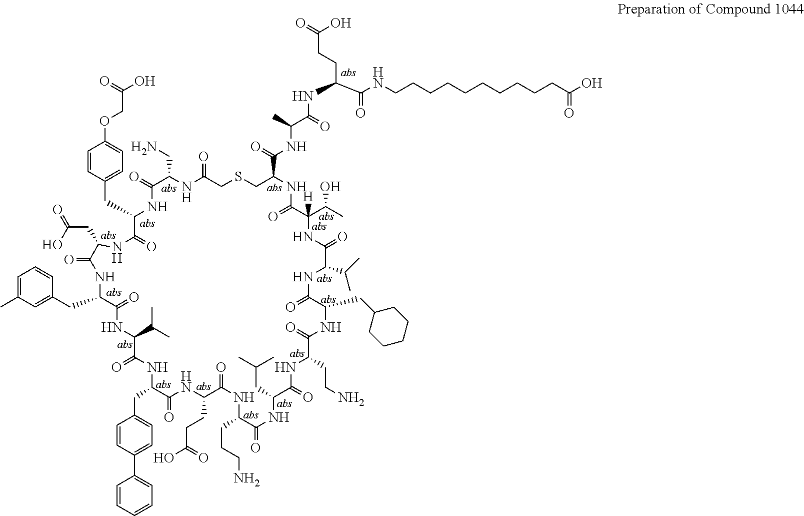 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1044 was prepared on a 100 μmol scale. The yield of the product was 35.1 mg, and its estimated purity by LCMS analysis was 95.6%. Analysis condition A: Retention time=1.42 min; ESI-MS(+) m/z [M+2H]²⁺: 1130.4.

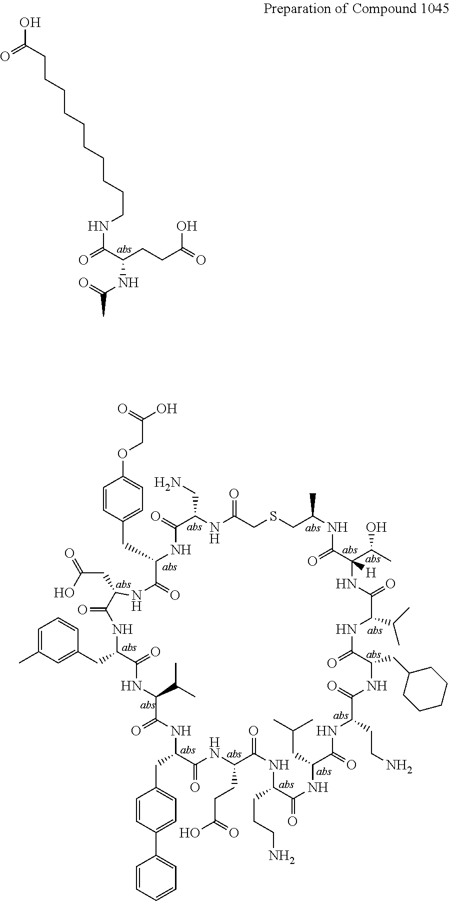

Compound 1045 was prepared on a 100 μmol scale. The yield of the product was 26.3 mg, and its estimated purity by LCMS analysis was 98.9%. Analysis condition B: Retention time=1.68 min; ESI-MS(+) m/z [M+3H]³⁺: 730.9.

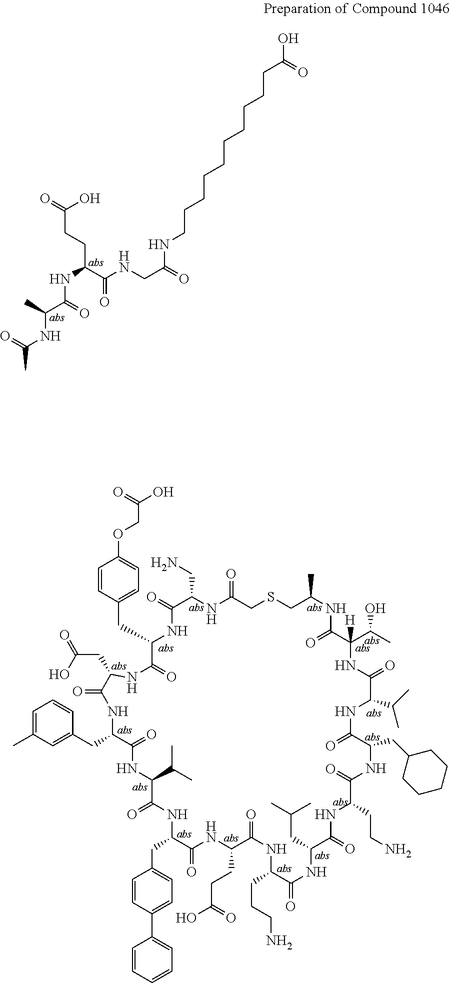

Compound 1046 was prepared on a 100 μmol scale. The yield of the product was 30.5 mg, and its estimated purity by LCMS analysis was 92.1%. Analysis condition A: Retention time=1.59 min; ESI-MS(+) m/z [M+3H]³⁺: 773.3.

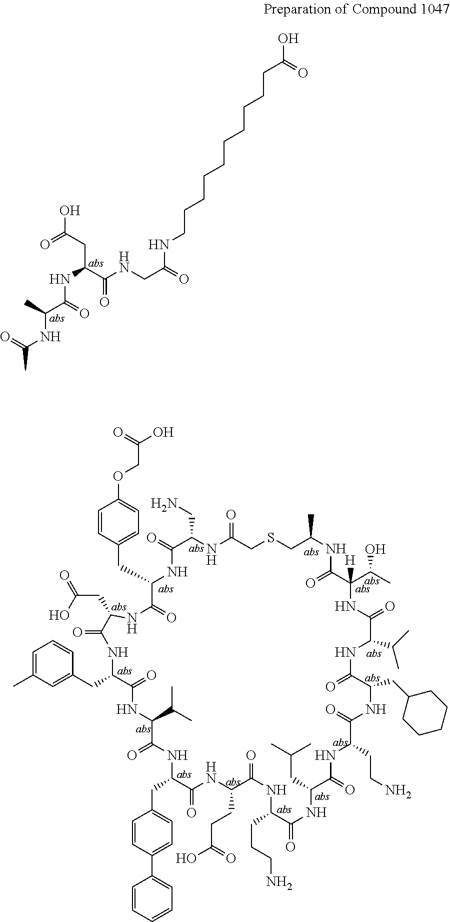

Compound 1047 was prepared on a 100 μmol scale. The yield of the product was 15 mg, and its estimated purity by LCMS analysis was 95.4%. Analysis condition B: Retention time=1.58 min; ESI-MS(+) m/z [M+3H]³⁺: 768.9.

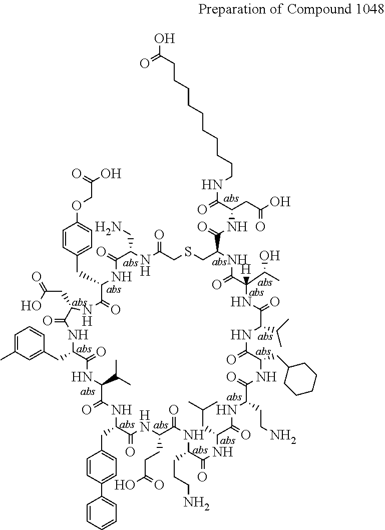 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1048 was prepared on a 100 μmol scale. The yield of the product was 37.4 mg, and its estimated purity by LCMS analysis was 90.2%. Analysis condition A: Retention time=1.42 min; ESI-MS(+) m/z [M+2H]²⁺:1088.

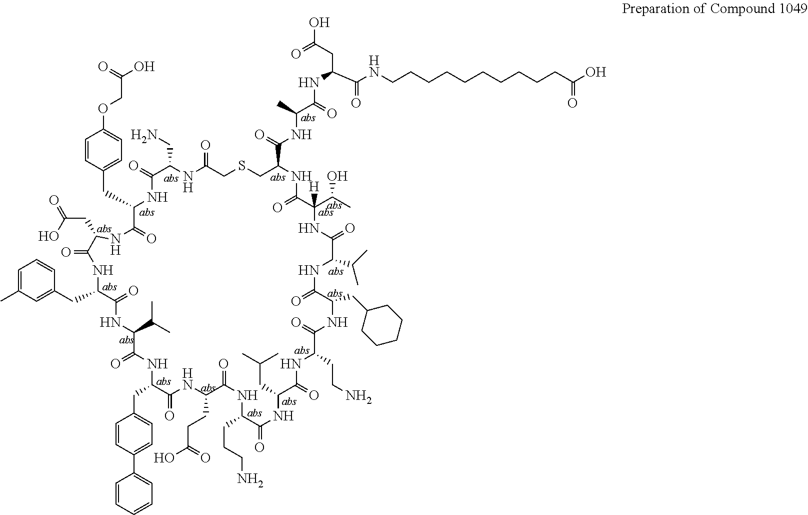 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1049 was prepared on a 100 μmol scale. The yield of the product was 9 mg, and its estimated purity by LCMS analysis was 92%. Analysis condition B: Retention time=1.56 min; ESI-MS(+) m/z [M+2H]²⁺: 1124.1.

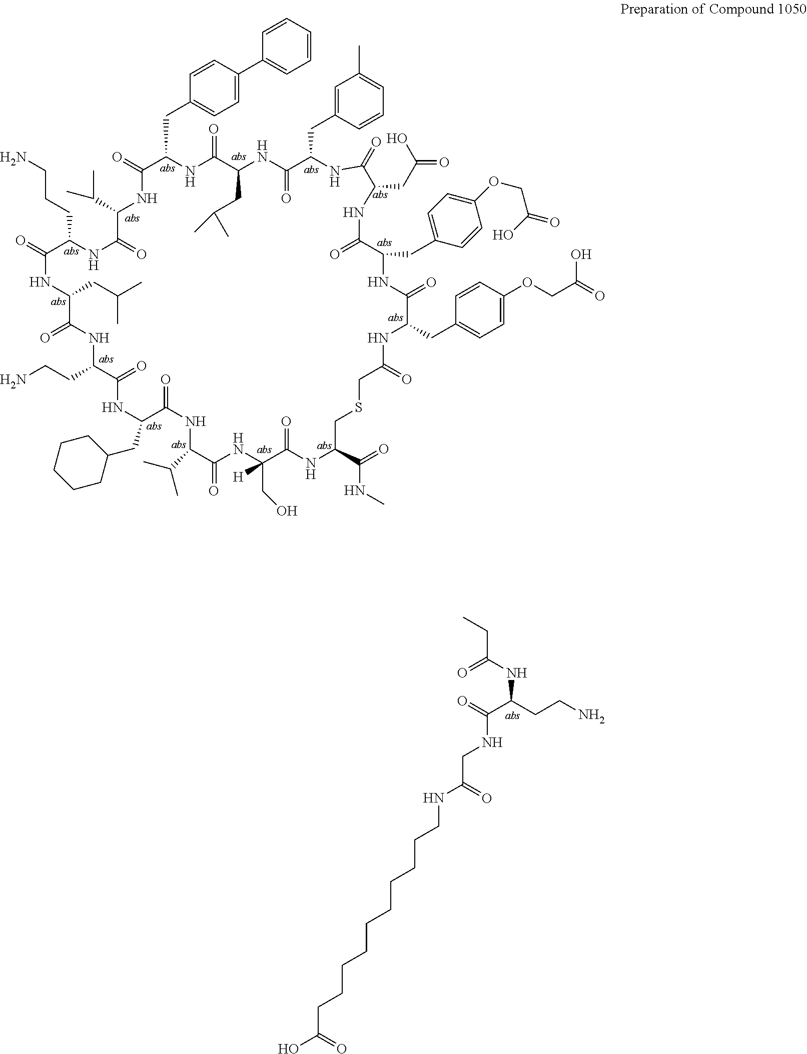 [SMILES: CNC(=O)[C@@H]1CSCC(=O)N[C@@H](Cc2ccc(OCC(=O)O)cc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](CC(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CO)C(=O)N1 ; CCC(=O)N[C@@H](CCN)C(=O)NCC(=O)NCCCCCCCCCCC(=O)O]

Compound 1050 was prepared on a 50 μmol scale. The yield of the product was 11.4 mg, and its estimated purity by LCMS analysis was 98.9%. Analysis condition A: Retention time=1.82 min; ESI-MS(+) m/z [M+2H]²⁺: 1190.3.

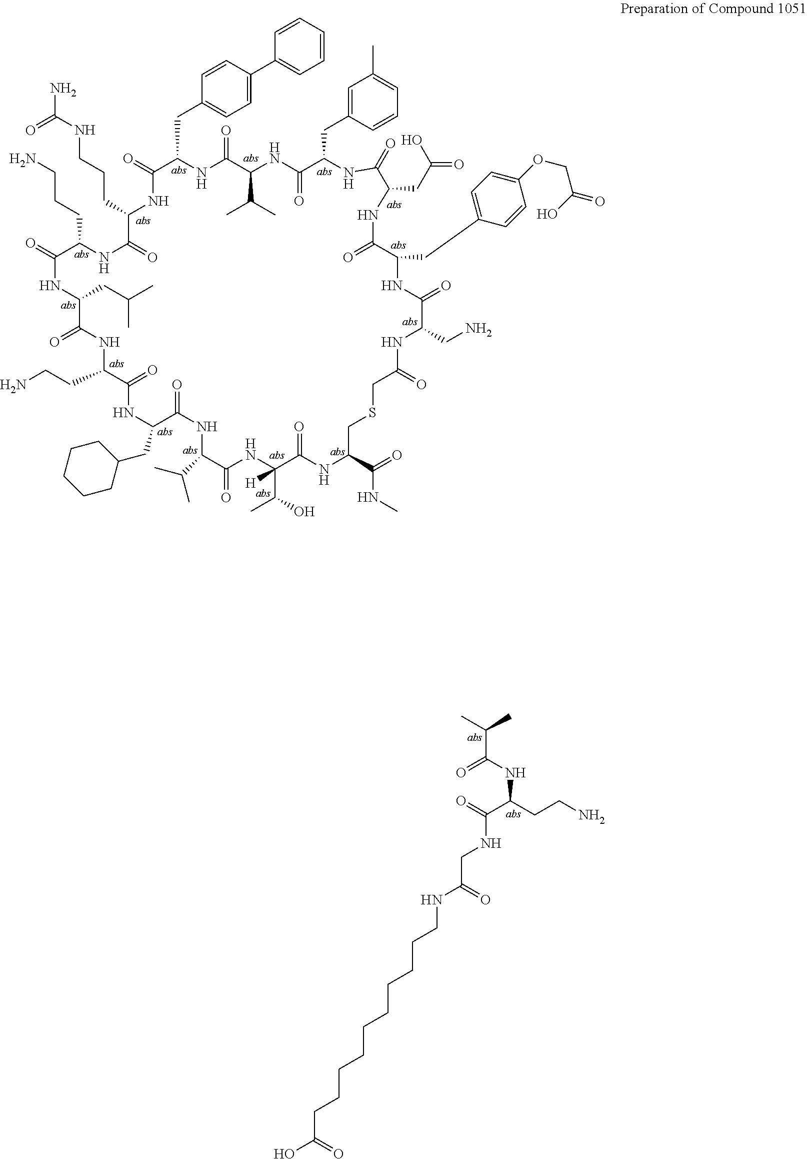

Compound 1051 was prepared on a 50 μmol scale. The yield of the product was 18.3 mg, and its estimated purity by LCMS analysis was 90.5%. Analysis condition B: Retention time=1.65 min; ESI-MS(+) m/z [M+2H]²⁺: 1159.2.

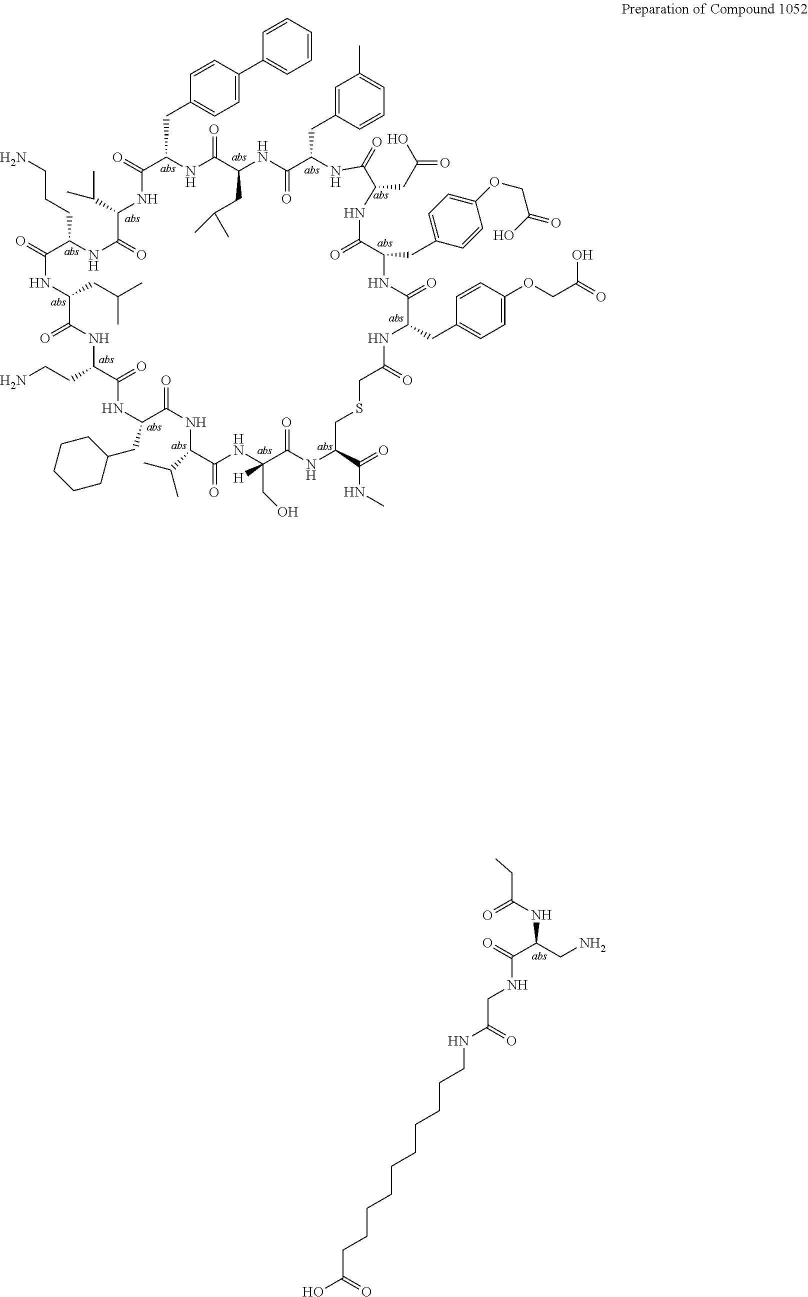 [SMILES: CNC(=O)[C@@H]1CSCC(=O)N[C@@H](Cc2ccc(OCC(=O)O)cc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](CC(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CO)C(=O)N1 ; CCC(=O)N[C@@H](CN)C(=O)NCC(=O)NCCCCCCCCCCC(=O)O]

Compound 1052 was prepared on a 50 μmol scale. The yield of the product was 18.7 mg, and its estimated purity by LCMS analysis was 93.9%. Analysis condition A: Retention time=2.09 min; ESI-MS(+) m/z [M+2H]²⁺: 1184.3.

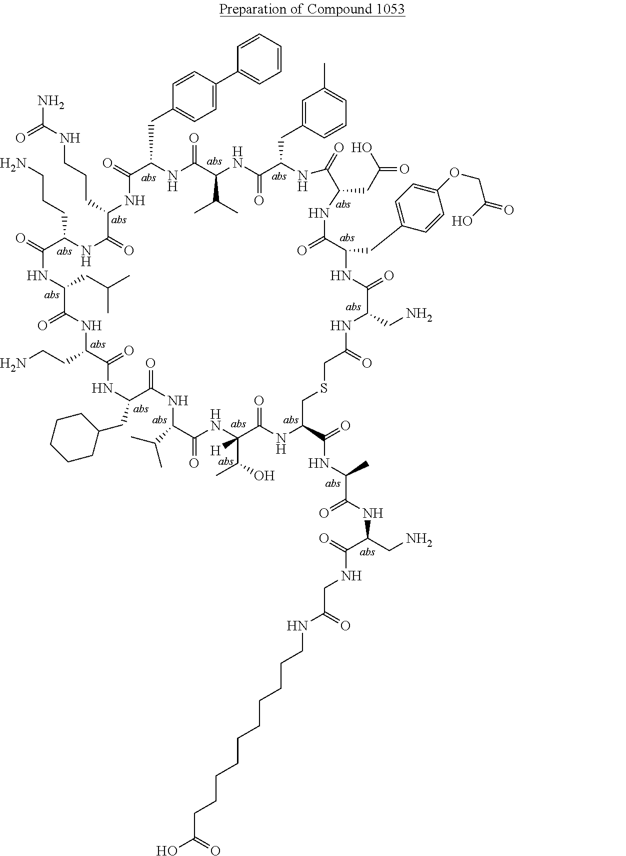 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CN)C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1053 was prepared on a 50 μmol scale. The yield of the product was 12.3 mg, and its estimated purity by LCMS analysis was 93.9%. Analysis condition A: Retention time=1.62 min; ESI-MS(+) m/z [M+2H]²⁺: 1152.

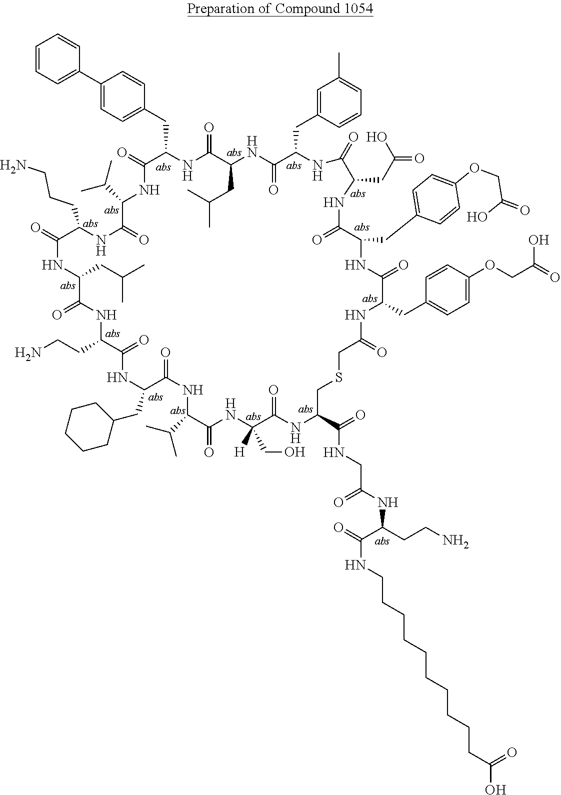 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1054 was prepared on a 50 μmol scale. The yield of the product was 14.2 mg, and its estimated purity by LCMS analysis was 100%. Analysis condition A: Retention time=2.06 min; ESI-MS(+) m/z [M+2H]²⁺: 1162.2.

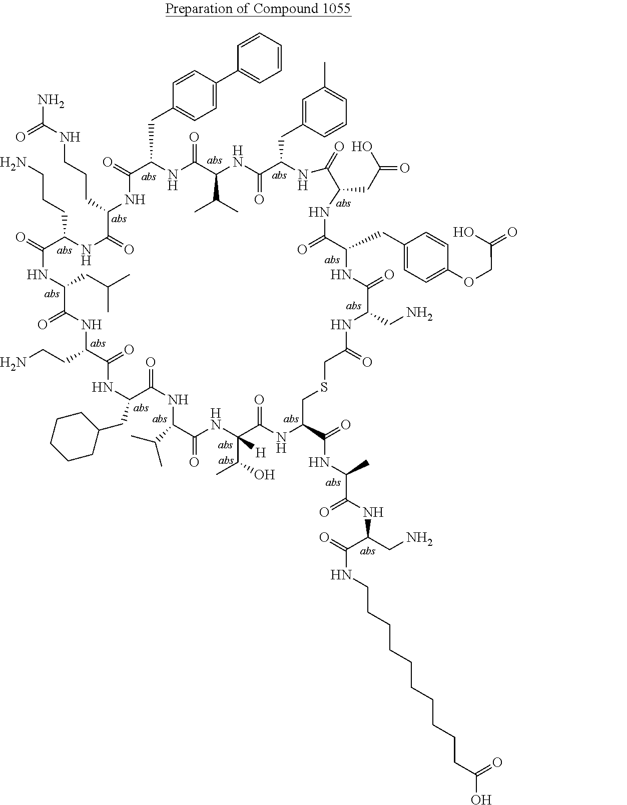 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1055 was prepared on a 50 μmol scale. The yield of the product was 5.4 mg, and its estimated purity by LCMS analysis was 95.3%. Analysis condition A: Retention time=1.93 min; ESI-MS(+) m/z [M+2H]²⁺: 1131.1.

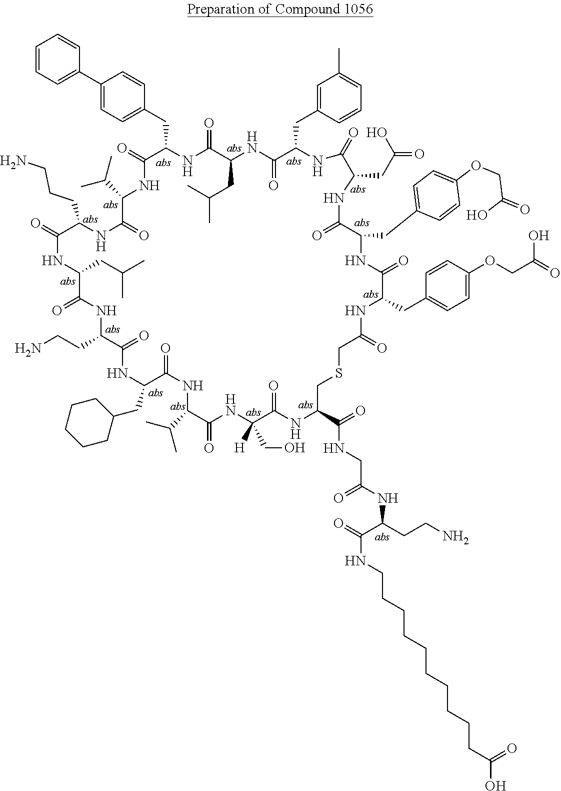 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1056 was prepared on a 50 μmol scale. The yield of the product was 41.1 mg, and its estimated purity by LCMS analysis was 97.7%. Analysis condition A: Retention time=1.8, 1.83 min; ESI-MS(+) m/z [M+2H]²⁺:1155.07, 1155.07.

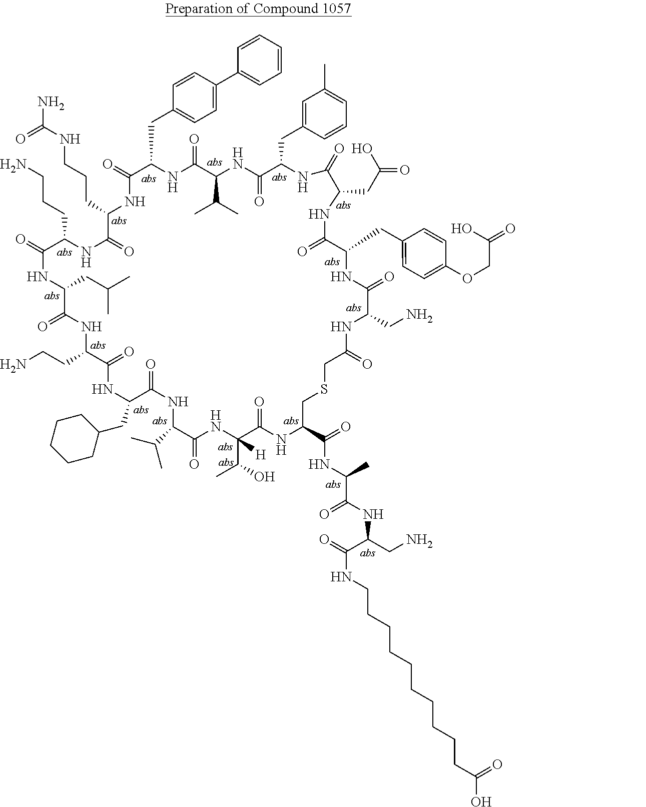 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1057 was prepared on a 50 μmol scale. The yield of the product was 6.3 mg, and its estimated purity by LCMS analysis was 92.8%. Analysis condition A: Retention time=1.63 min; ESI-MS(+) m/z [M+2H]²⁺: 1123.3.

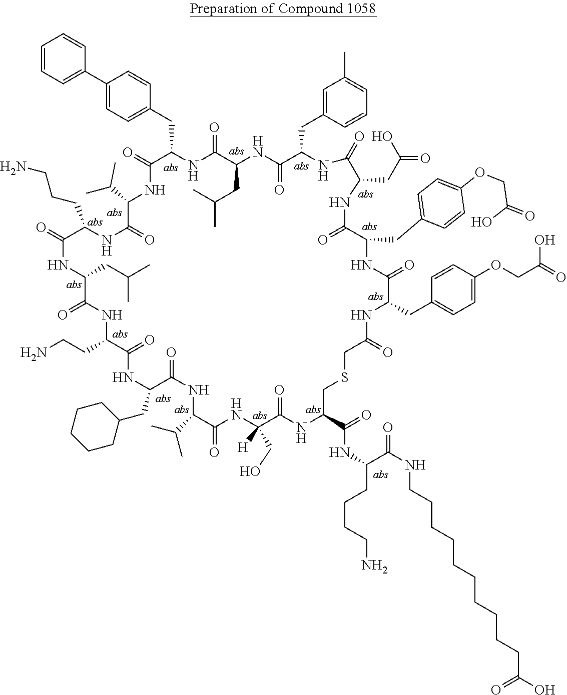 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCCCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1058 was prepared on a 50 μmol scale. The yield of the product was 1.8 mg, and its estimated purity by LCMS analysis was 100%. Analysis condition A: Retention time=1.79 min; ESI-MS(+) m/z [M+2H]²⁺: 1147.2.

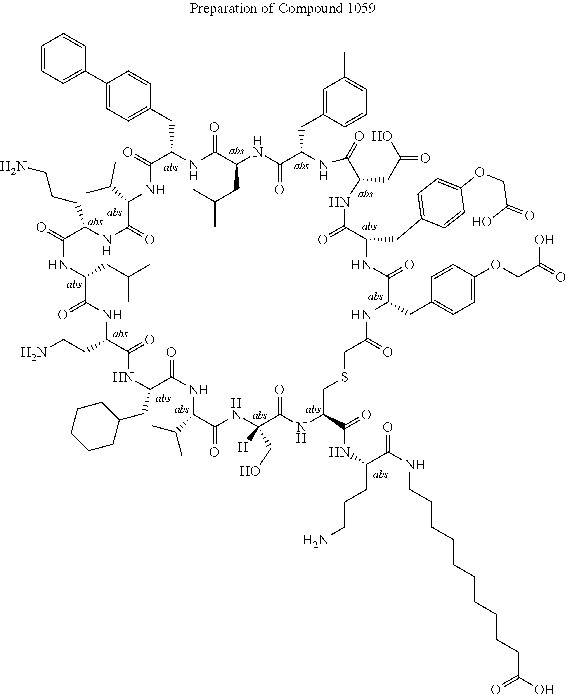 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1059 was prepared on a 50 μmol scale. The yield of the product was 9.3 mg, and its estimated purity by LCMS analysis was 90.2%. Analysis condition B: Retention time=1.68 min; ESI-MS(+) m/z [M+2H]²⁺: 1140.3.

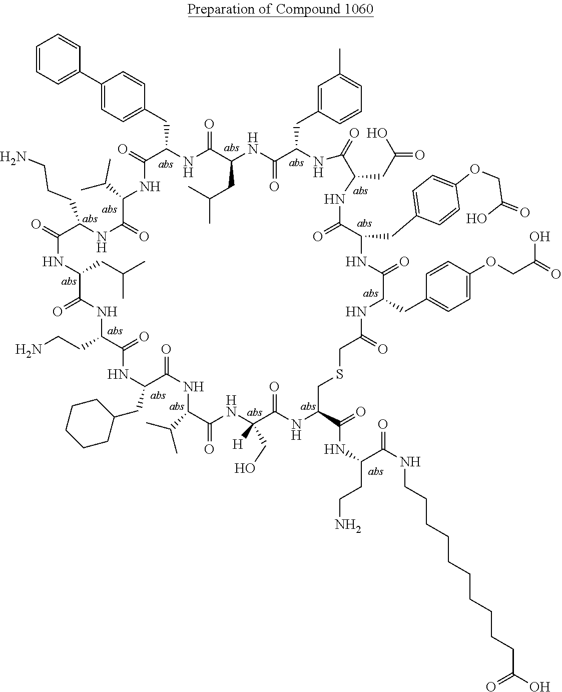 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1060 was prepared on a 50 μmol scale. The yield of the product was 13.2 mg, and its estimated purity by LCMS analysis was 80%. Analysis condition A: Retention time=1.81 min; ESI-MS(+) m/z [M+2H]²⁺: 1133.3.

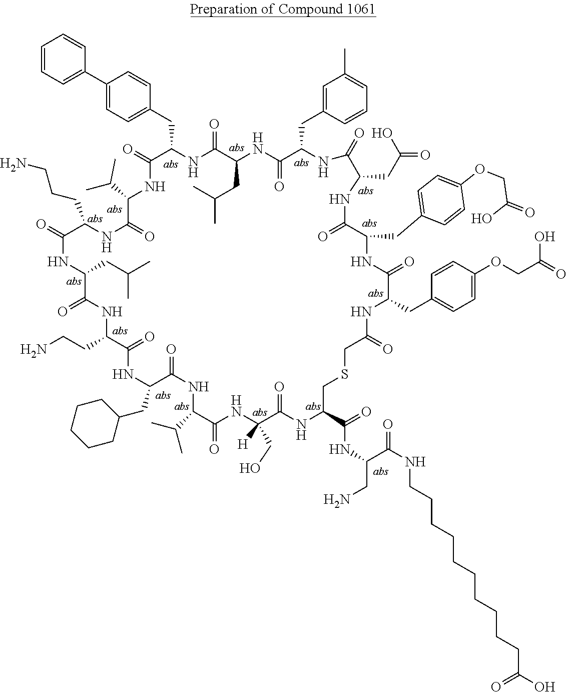 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1061 was prepared on a 50 μmol scale. The yield of the product was 2.8 mg, and its estimated purity by LCMS analysis was 90.7%. Analysis condition A: Retention time=1.78 min; ESI-MS(+) m/z [M+3H]³⁺: 751.4.

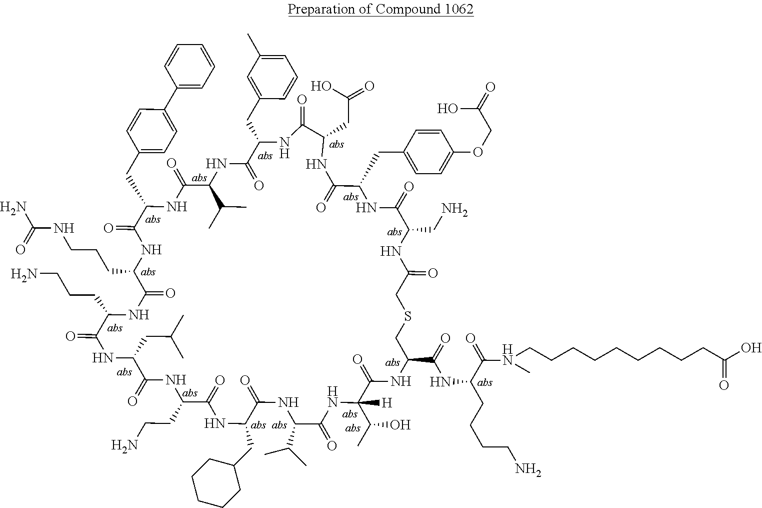

Compound 1062 was prepared on a 50 μmol scale. The yield of the product was 11.6 mg, and its estimated purity by LCMS analysis was 90.4%. Analysis condition B: Retention time=1.6 min; ESI-MS(+) m/z [M+2H]²⁺: 1108.9.

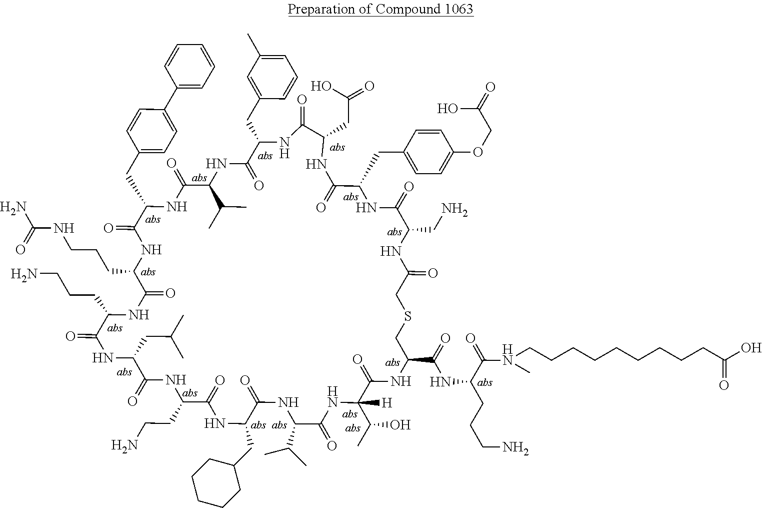

Compound 1063 was prepared on a 50 μmol scale. The yield of the product was 8.9 mg, and its estimated purity by LCMS analysis was 90.9%. Analysis condition A: Retention time=1.57 min; ESI-MS(+) m/z [M+2H]²⁺: 1102.2.

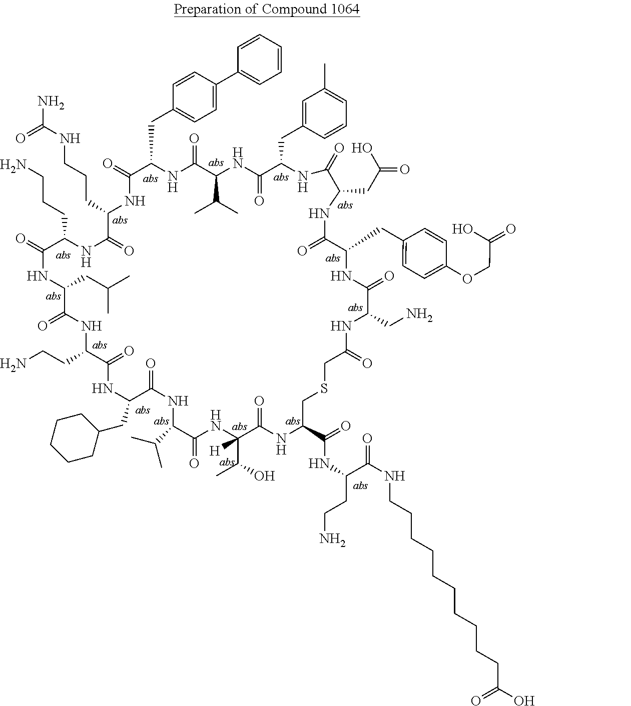 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1064 was prepared on a 50 μmol scale. The yield of the product was 13.6 mg, and its estimated purity by LCMS analysis was 100%. Analysis condition A: Retention time=1.9 min; ESI-MS(+) m/z [M+2H]²⁺: 1095.1.

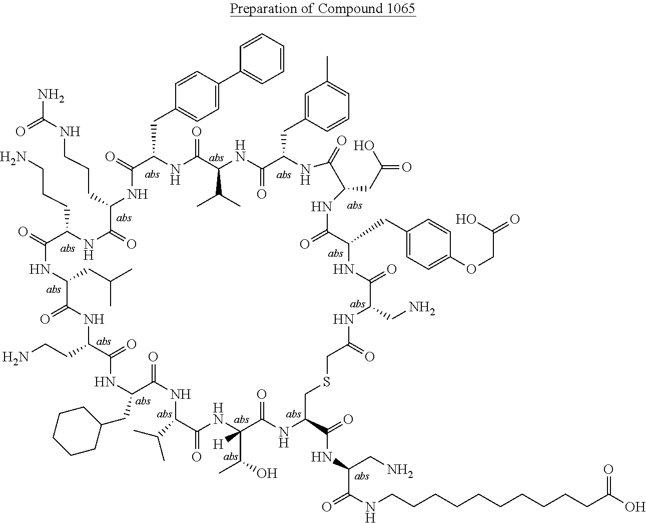 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1065 was prepared on a 50 μmol scale. The yield of the product was 9.8 mg, and its estimated purity by LCMS analysis was 94.4%. Analysis condition B: Retention time=1.65 min; ESI-MS(+) m/z [M+2H]²⁺: 1088.1.

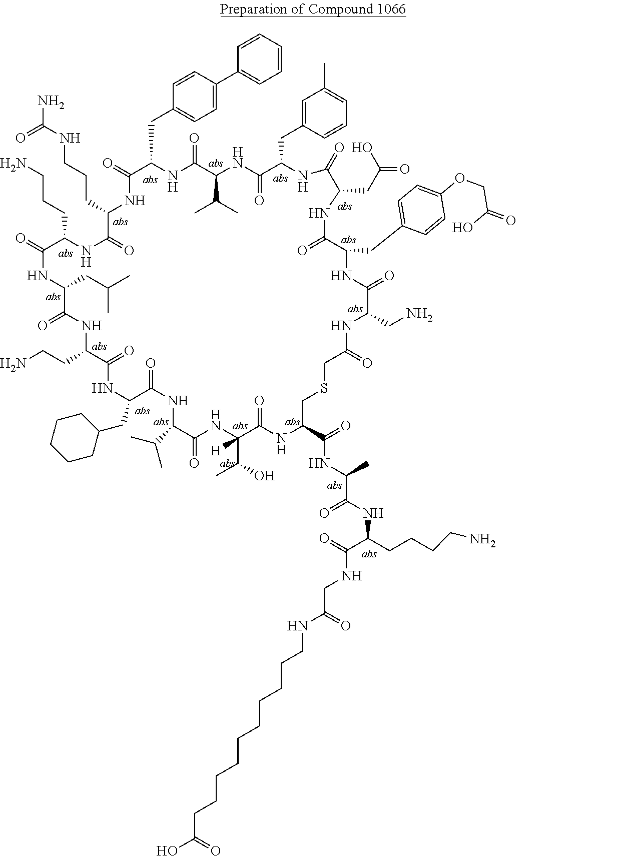 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCCCN)C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1066 was prepared on a 50 μmol scale. The yield of the product was 16.3 mg, and its estimated purity by LCMS analysis was 93.2%. Analysis condition B: Retention time=1.63 min; ESI-MS(+) m/z [M+2H]²⁺: 1172.9.

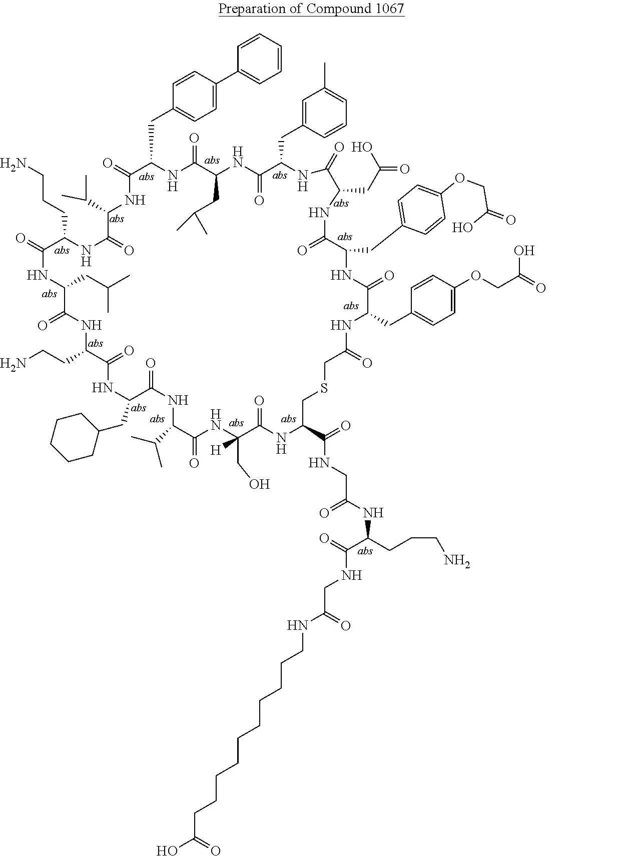 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(=O)N[C@@H](CCCN)C(=O)NCC(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1067 was prepared on a 50 μmol scale. The yield of the product was 6.2 mg, and its estimated purity by LCMS analysis was 98%. Analysis condition A: Retention time=1.93 min; ESI-MS(+) m/z [M+3H]³⁺: 798.9.

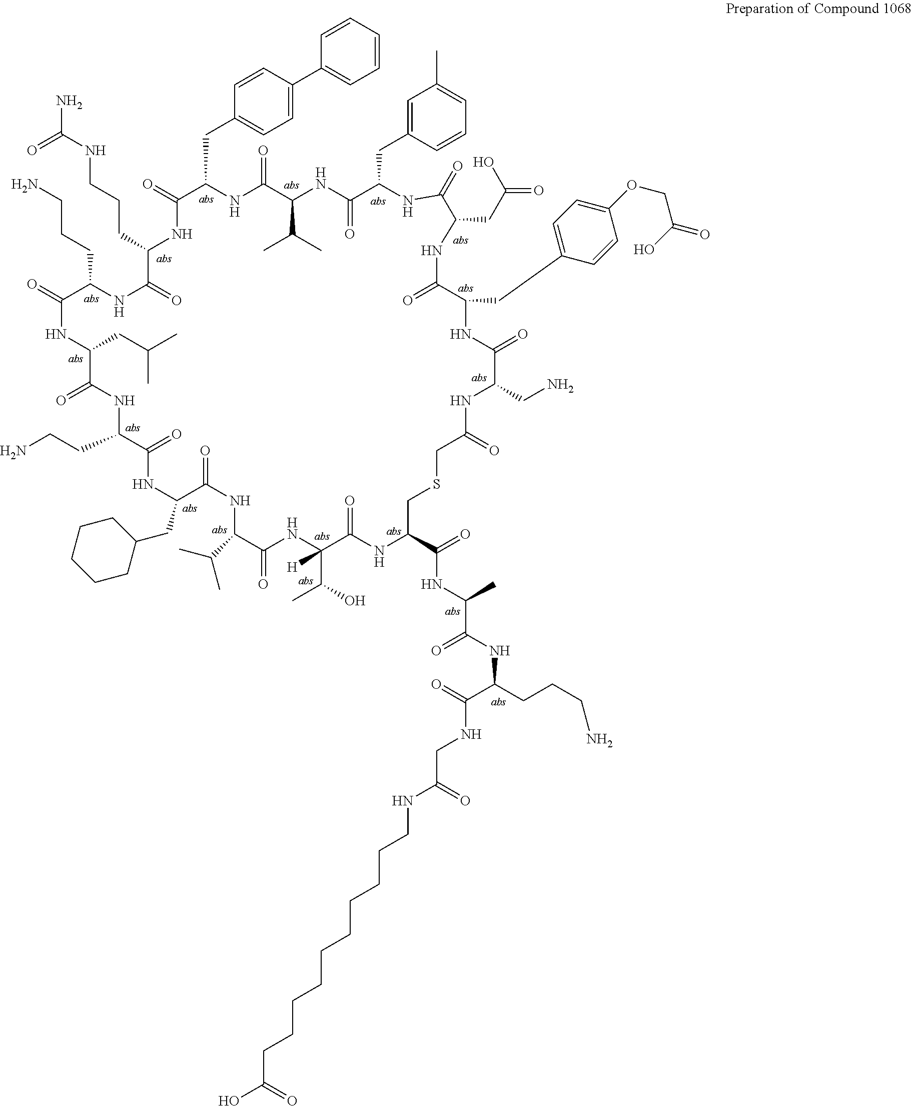 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCCN)C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1068 was prepared on a 50 μmol scale. The yield of the product was 11.4 mg, and its estimated purity by LCMS analysis was 100%. Analysis condition A: Retention time=1.59 min; ESI-MS(+) m/z [M+2H]²⁺: 1165.8.

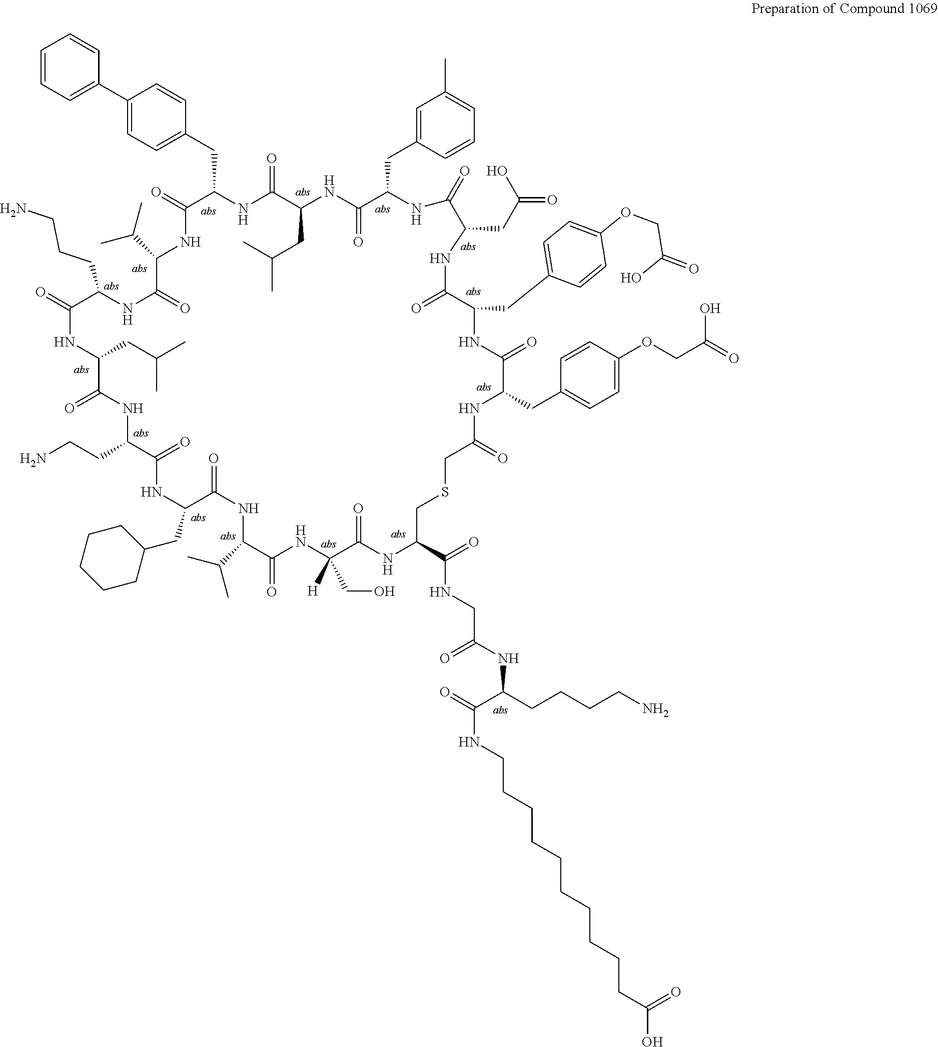 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(=O)N[C@@H](CCCCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1069 was prepared on a 50 μmol scale. The yield of the product was 16.7 mg, and its estimated purity by LCMS analysis was 90.1%. Analysis condition A: Retention time=2.09 min; ESI-MS(+) m/z [M+2H]²⁺: 1176.

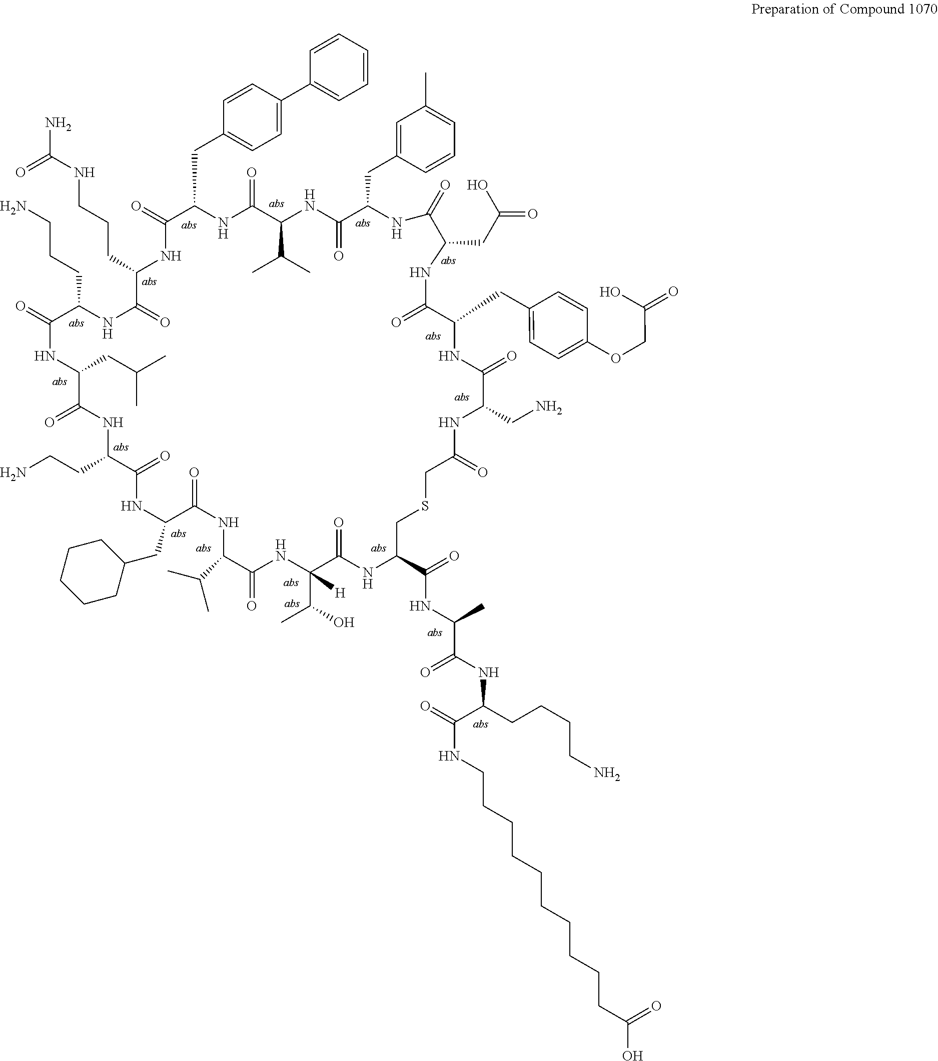 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1070 was prepared on a 50 μmol scale. The yield of the product was 17.9 mg, and its estimated purity by LCMS analysis was 95.8%. Analysis condition B: Retention time=1.55 min; ESI-MS(+) m/z [M+3H]³⁺: 763.2.

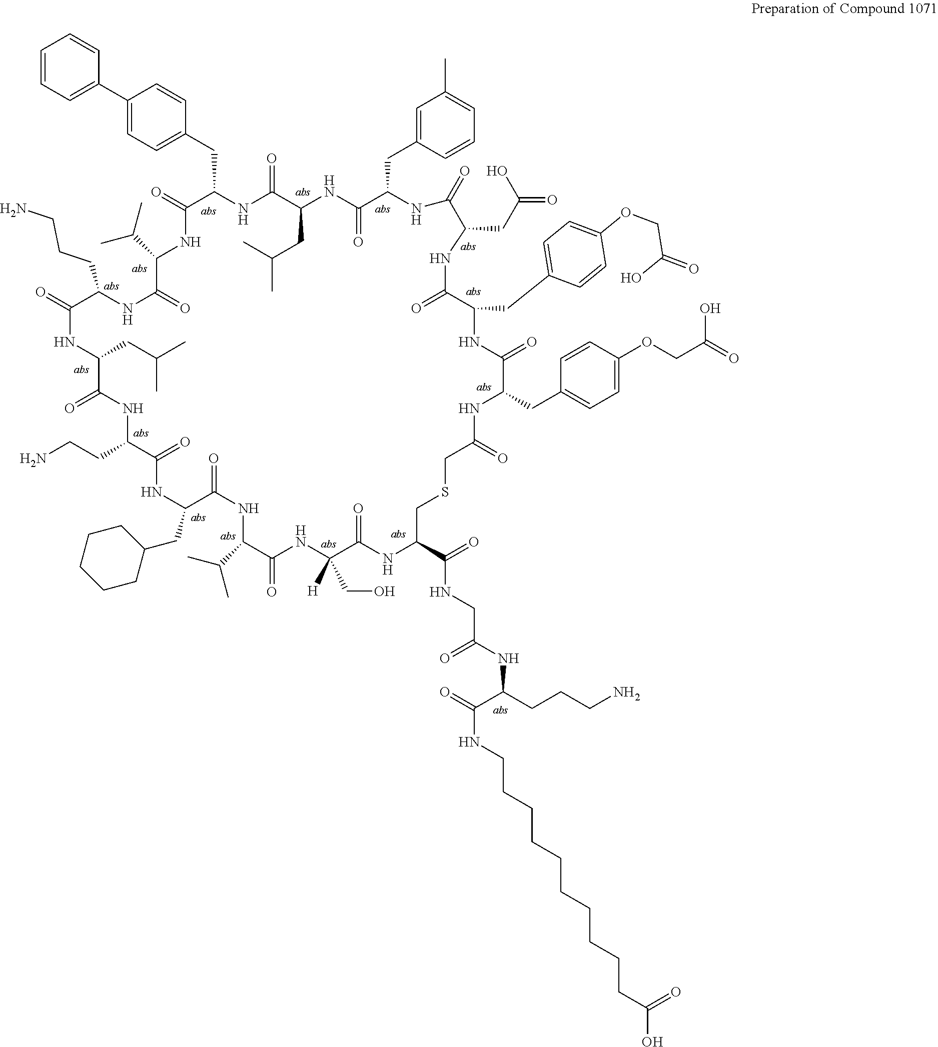 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(=O)N[C@@H](CCCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1071 was prepared on a 50 μmol scale. The yield of the product was 16.6 mg, and its estimated purity by LCMS analysis was 90.5%. Analysis condition A: Retention time=1.88 min; ESI-MS(+) m/z [M+2H]²⁺: 1168.9.

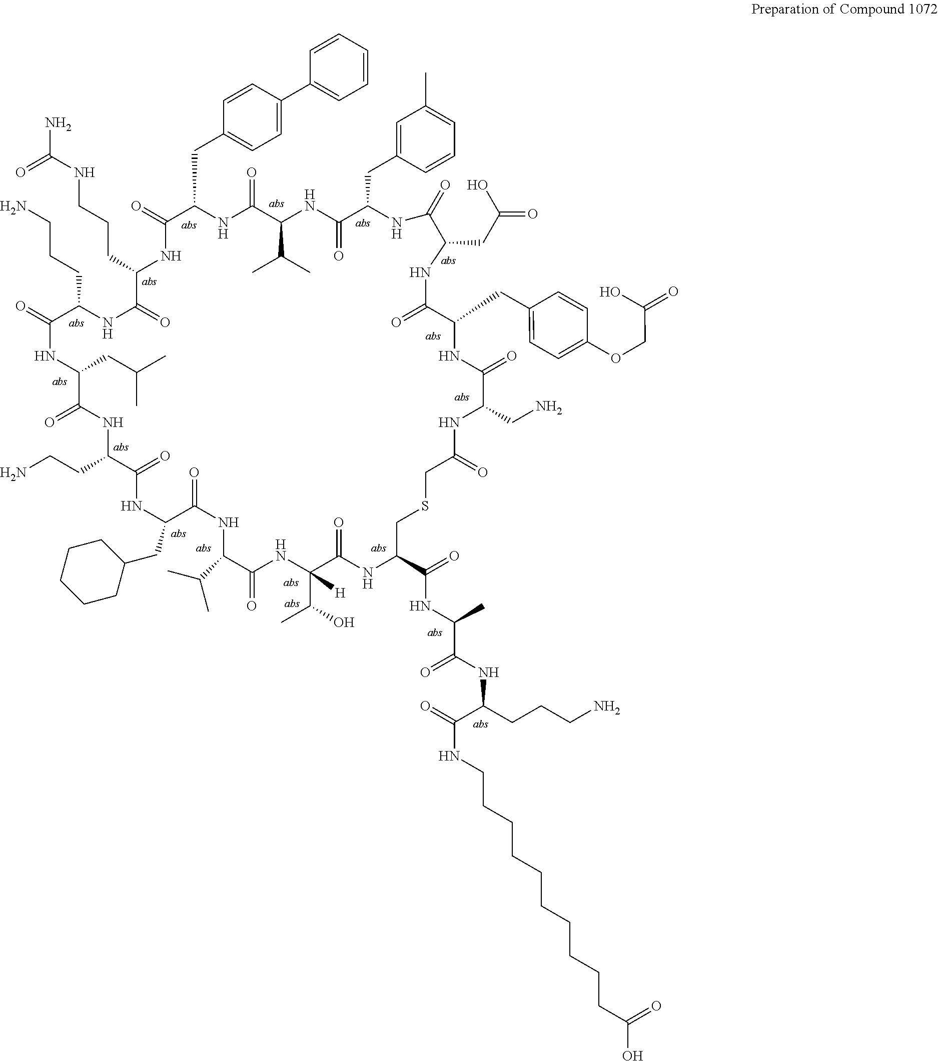 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1072 was prepared on a 50 μmol scale. The yield of the product was 22.4 mg, and its estimated purity by LCMS analysis was 90.7%. Analysis condition A: Retention time=1.89 min; ESI-MS(+) m/z [M+2H]²⁺: 1138.

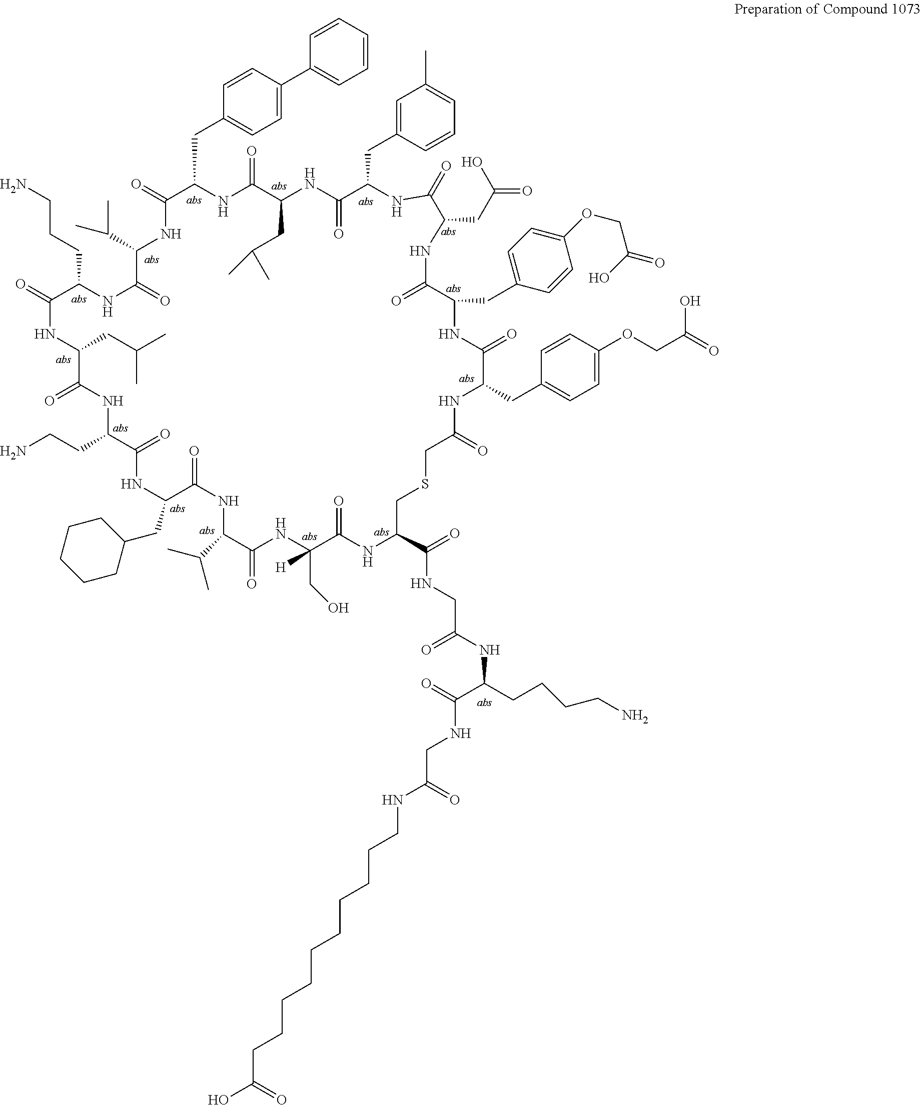 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(=O)N[C@@H](CCCCN)C(=O)NCC(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1073 was prepared on a 50 μmol scale. The yield of the product was 15.3 mg, and its estimated purity by LCMS analysis was 95.8%. Analysis condition A: Retention time=1.78 min; ESI-MS(+) m/z [M+3H]³⁺: 803.2.

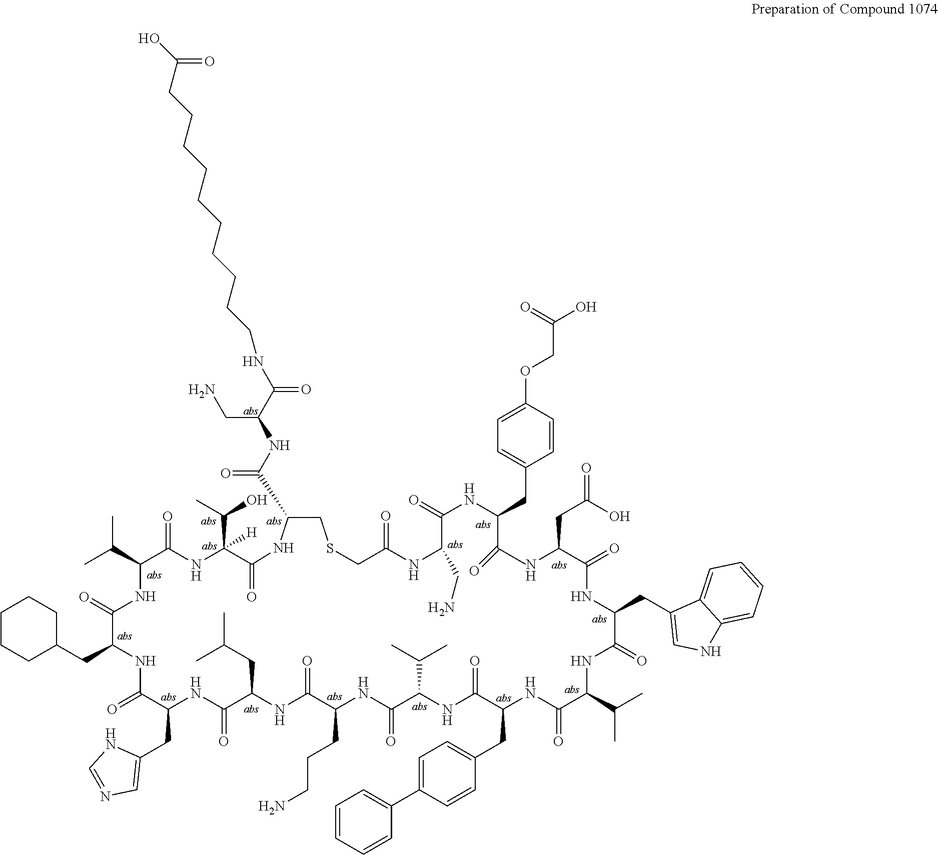 [SMILES: CC(=O)[C@H](Cc1ccc(OCC(=O)O)cc1)NC(=O)[C@H](CN)NC(=O)CSC[C@H](NC(=O)[C@@H](NC(=O)[C@@H](NC(=O)[C@H](CC1CCCCC1)NC(=O)[C@H](Cc1cnc[nH]1)NC(=O)[C@@H](CC(C)C)NC(=O)[C@H](CCCN)NC(=O)[C@@H](NC(=O)[C@H](Cc1ccc(-c2ccccc2)cc1)NC(=O)[C@@H](NC(=O)[C@H](Cc1c[nH]c2ccccc12)NC(=O)[C@@H](N)CC(=O)O)C(C)C)C(C)C)C(C)C)[C@@H](C)O)C(=O)N[C@@H](CN)C(=O)NCCCCCCCCCCC(=O)O]

Compound 1074 was prepared on a 50 μmol scale. The yield of the product was 18.6 mg, and its estimated purity by LCMS analysis was 83.8%. Analysis condition B: Retention time=1.4 min; ESI-MS(+) m/z [M+2H]²⁺: 1089.4.

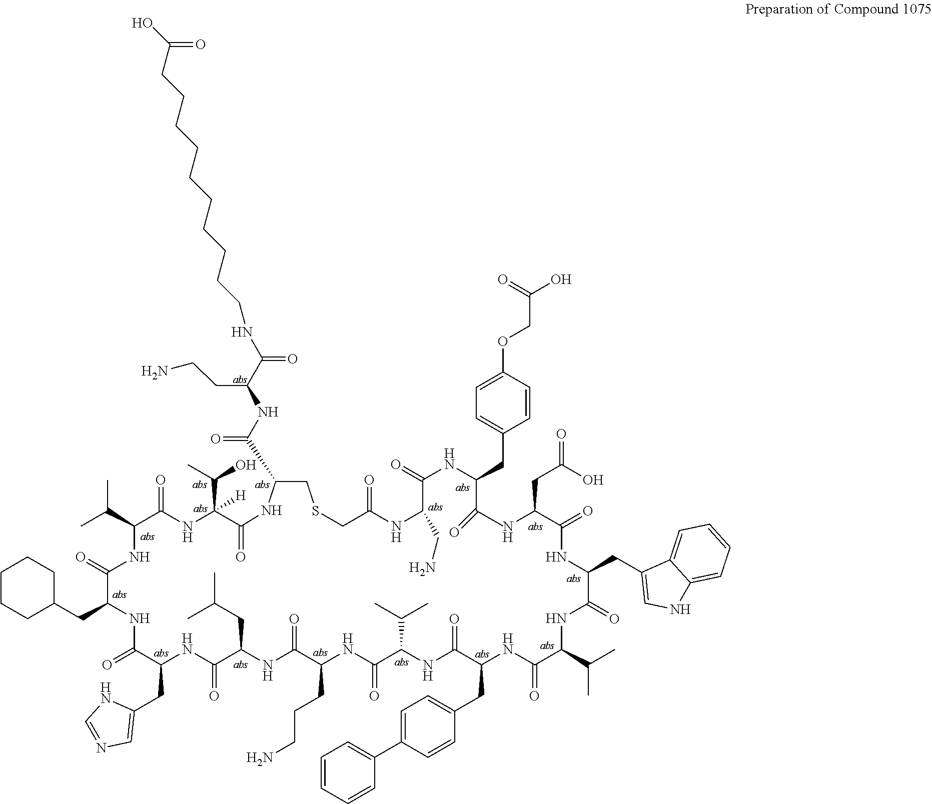 [SMILES: CC(=O)[C@H](Cc1ccc(OCC(=O)O)cc1)NC(=O)[C@H](CN)NC(=O)CSC[C@H](NC(=O)[C@@H](NC(=O)[C@@H](NC(=O)[C@H](CC1CCCCC1)NC(=O)[C@H](Cc1cnc[nH]1)NC(=O)[C@@H](CC(C)C)NC(=O)[C@H](CCCN)NC(=O)[C@@H](NC(=O)[C@H](Cc1ccc(-c2ccccc2)cc1)NC(=O)[C@@H](NC(=O)[C@H](Cc1c[nH]c2ccccc12)NC(=O)[C@@H](N)CC(=O)O)C(C)C)C(C)C)C(C)C)[C@@H](C)O)C(=O)N[C@@H](CCN)C(=O)NCCCCCCCCCCC(=O)O]

Compound 1075 was prepared on a 50 μmol scale. The yield of the product was 30.7 mg, and its estimated purity by LCMS analysis was 93.2%. Analysis condition A: Retention time=1.68 min; ESI-MS(+) m/z [M+2H]²⁺: 1097.

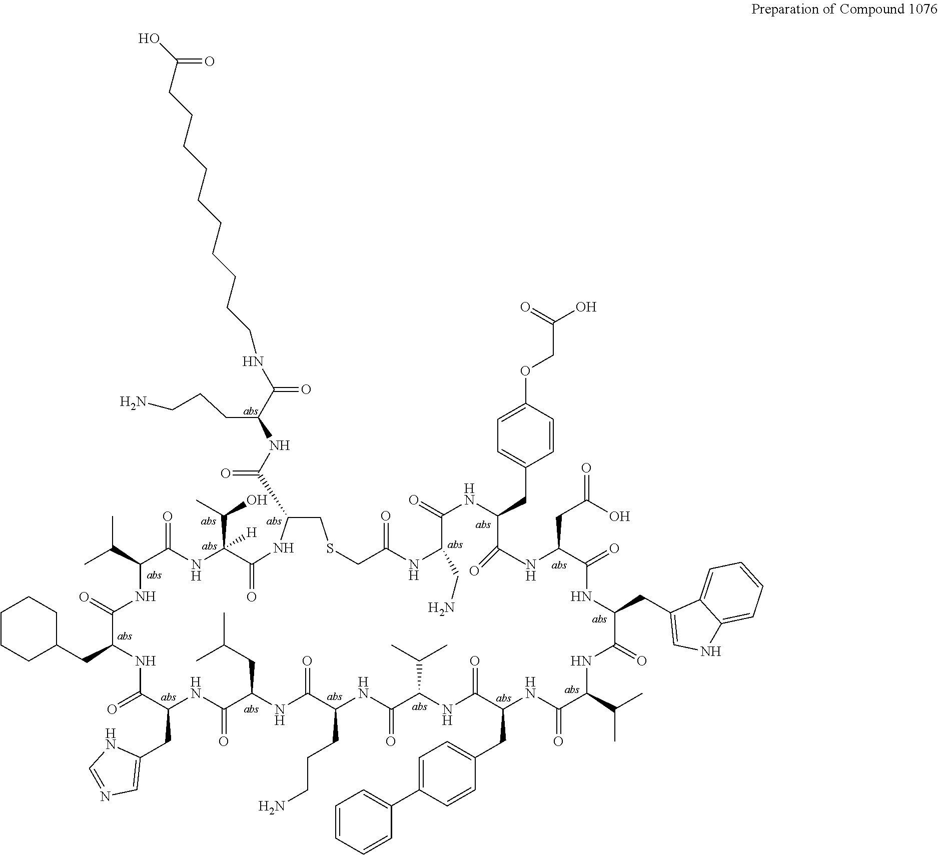 [SMILES: CC(=O)[C@H](Cc1ccc(OCC(=O)O)cc1)NC(=O)[C@H](CN)NC(=O)CSC[C@H](NC(=O)[C@@H](NC(=O)[C@@H](NC(=O)[C@H](CC1CCCCC1)NC(=O)[C@H](Cc1cnc[nH]1)NC(=O)[C@@H](CC(C)C)NC(=O)[C@H](CCCN)NC(=O)[C@@H](NC(=O)[C@H](Cc1ccc(-c2ccccc2)cc1)NC(=O)[C@@H](NC(=O)[C@H](Cc1c[nH]c2ccccc12)NC(=O)[C@@H](N)CC(=O)O)C(C)C)C(C)C)C(C)C)[C@@H](C)O)C(=O)N[C@@H](CCCN)C(=O)NCCCCCCCCCCC(=O)O]

Compound 1076 was prepared on a 50 μmol scale. The yield of the product was 31.4 mg, and its estimated purity by LCMS analysis was 91.7%. Analysis condition A: Retention time=1.68 min; ESI-MS(+) m/z [M+2H]²⁺: 1104.2.

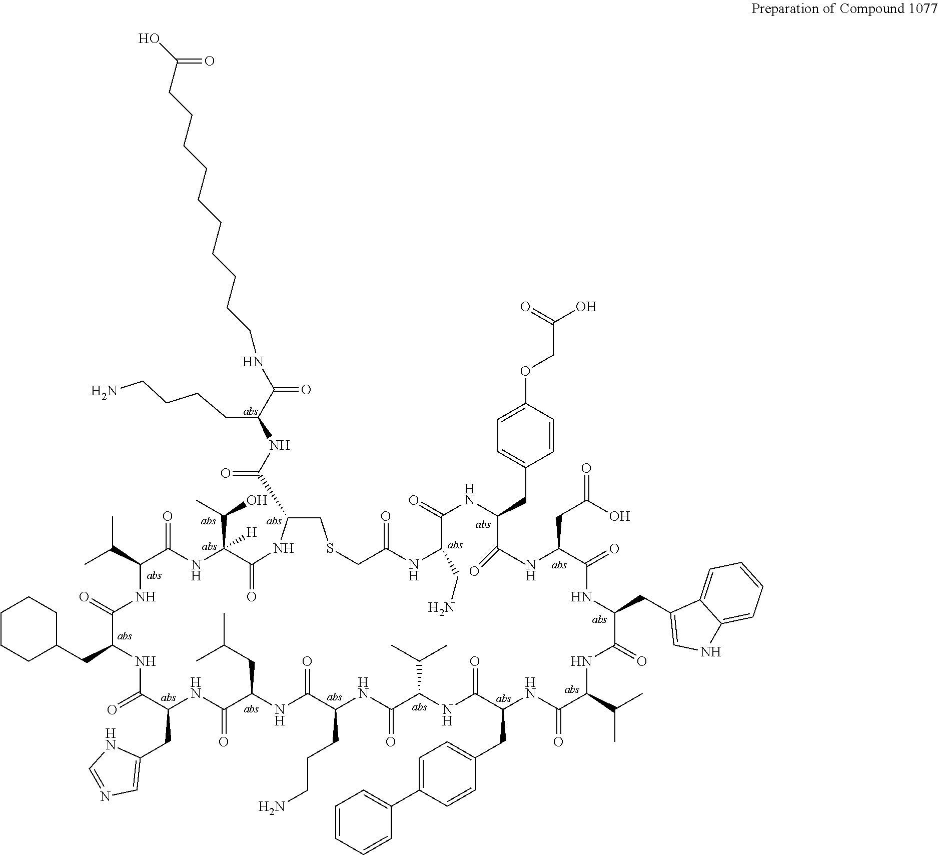 [SMILES: CC(=O)[C@H](Cc1ccc(OCC(=O)O)cc1)NC(=O)[C@H](CN)NC(=O)CSC[C@H](NC(=O)[C@@H](NC(=O)[C@@H](NC(=O)[C@H](CC1CCCCC1)NC(=O)[C@H](Cc1cnc[nH]1)NC(=O)[C@@H](CC(C)C)NC(=O)[C@H](CCCN)NC(=O)[C@@H](NC(=O)[C@H](Cc1ccc(-c2ccccc2)cc1)NC(=O)[C@@H](NC(=O)[C@H](Cc1c[nH]c2ccccc12)NC(=O)[C@@H](N)CC(=O)O)C(C)C)C(C)C)C(C)C)[C@@H](C)O)C(=O)N[C@@H](CCCCN)C(=O)NCCCCCCCCCCC(=O)O]

Compound 1077 was prepared on a 50 μmol scale. The yield of the product was 10.7 mg, and its estimated purity by LCMS analysis was 93.1%. Analysis condition B: Retention time=1.37 min; ESI-MS(+) m/z [M+2H]²⁺: 1110.3.

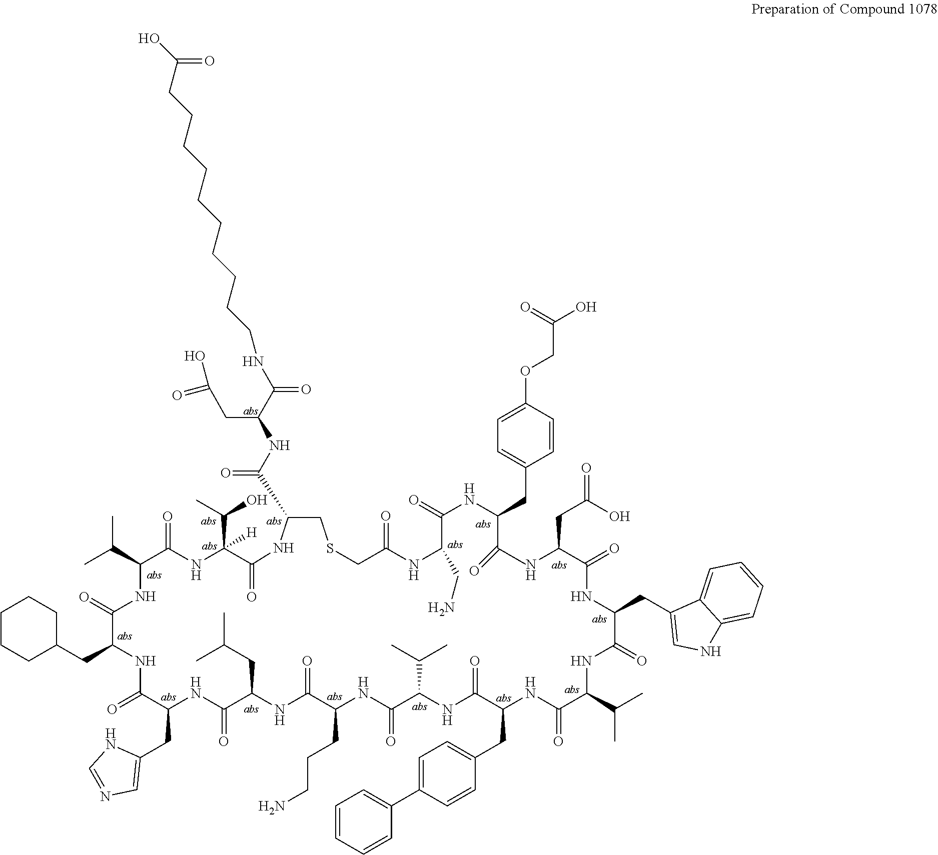 [SMILES: CC(=O)[C@H](Cc1ccc(OCC(=O)O)cc1)NC(=O)[C@H](CN)NC(=O)CSC[C@H](NC(=O)[C@@H](NC(=O)[C@@H](NC(=O)[C@H](CC1CCCCC1)NC(=O)[C@H](Cc1cnc[nH]1)NC(=O)[C@@H](CC(C)C)NC(=O)[C@H](CCCN)NC(=O)[C@@H](NC(=O)[C@H](Cc1ccc(-c2ccccc2)cc1)NC(=O)[C@@H](NC(=O)[C@H](Cc1c[nH]c2ccccc12)NC(=O)[C@@H](N)CC(=O)O)C(C)C)C(C)C)C(C)C)[C@@H](C)O)C(=O)N[C@@H](CC(=O)O)C(=O)NCCCCCCCCCCC(=O)O]

Compound 1078 was prepared on a 50 μmol scale. The yield of the product was 4.4 mg, and its estimated purity by LCMS analysis was 86.1%. Analysis condition B: Retention time=1.63 min; ESI-MS(+) m/z [M+3H]³⁺: 736.4.

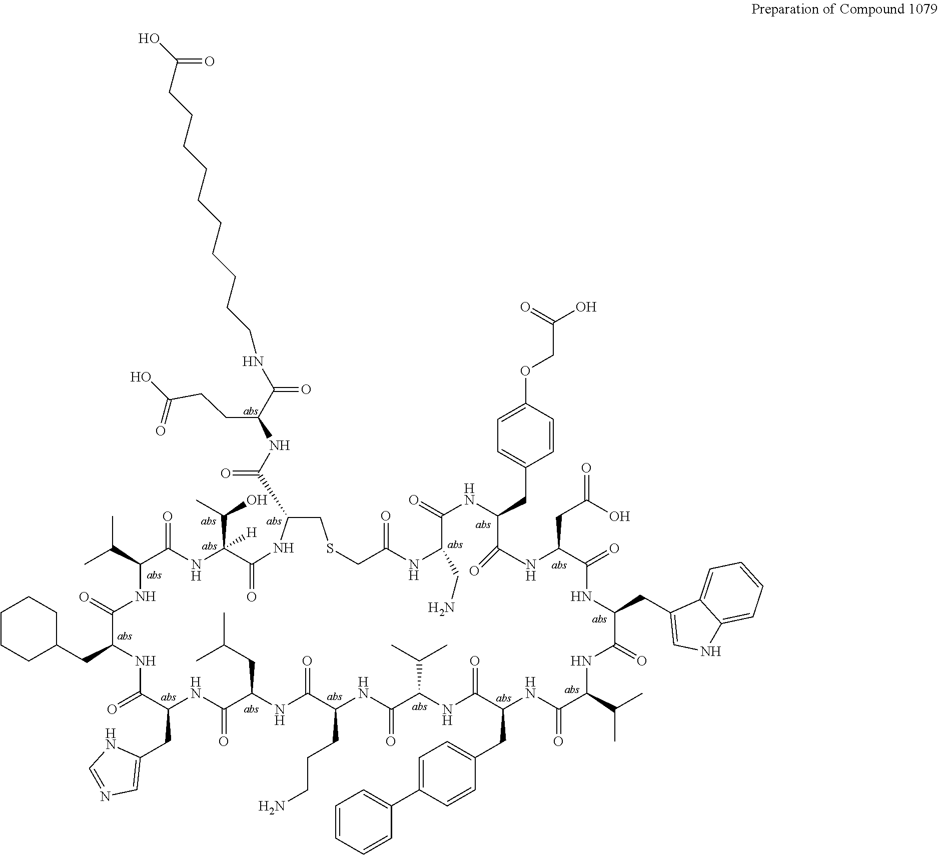 [SMILES: CC(=O)[C@H](Cc1ccc(OCC(=O)O)cc1)NC(=O)[C@H](CN)NC(=O)CSC[C@H](NC(=O)[C@@H](NC(=O)[C@@H](NC(=O)[C@H](CC1CCCCC1)NC(=O)[C@H](Cc1cnc[nH]1)NC(=O)[C@@H](CC(C)C)NC(=O)[C@H](CCCN)NC(=O)[C@@H](NC(=O)[C@H](Cc1ccc(-c2ccccc2)cc1)NC(=O)[C@@H](NC(=O)[C@H](Cc1c[nH]c2ccccc12)NC(=O)[C@@H](N)CC(=O)O)C(C)C)C(C)C)C(C)C)[C@@H](C)O)C(=O)N[C@@H](CCC(=O)O)C(=O)NCCCCCCCCCCC(=O)O]

Compound 1079 was prepared on a 50 μmol scale. The yield of the product was 6.3 mg, and its estimated purity by LCMS analysis was 90.2%. Analysis condition A: Retention time=1.72 min; ESI-MS(+) m/z [M+2H]²⁺: 1111.3.

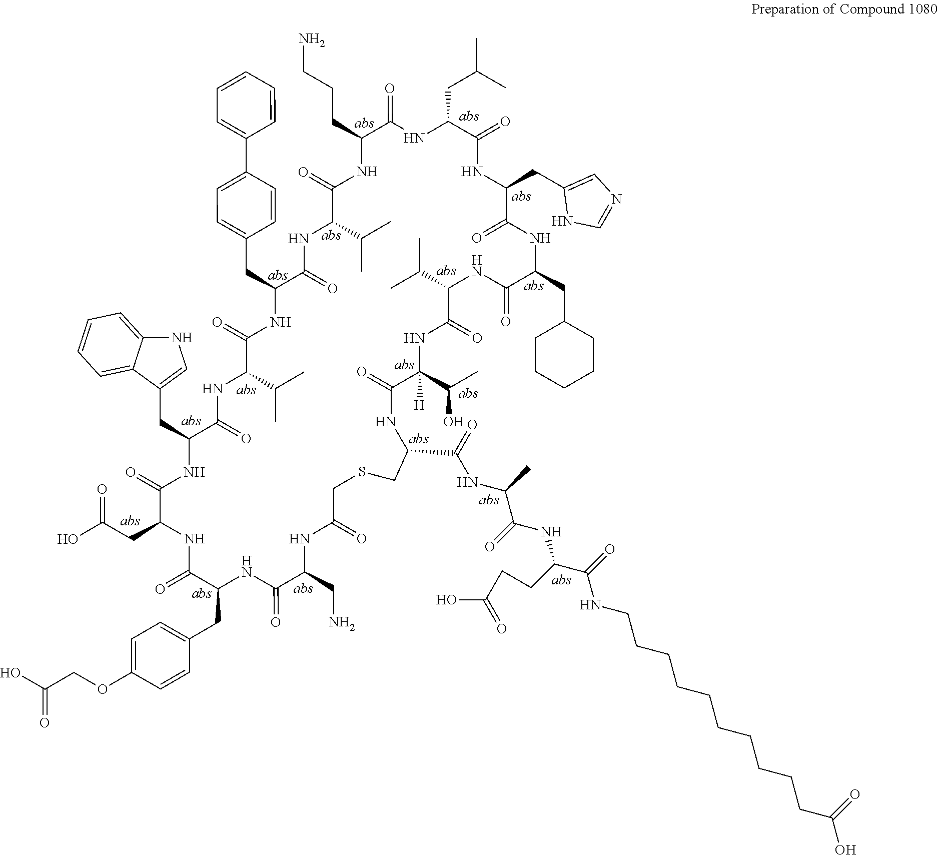 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1080 was prepared on a 50 μmol scale. The yield of the product was 3.3 mg, and its estimated purity by LCMS analysis was 90.4%. Analysis condition A: Retention time=1.58 min; ESI-MS(+) m/z [M+2H]²⁺: 1147.1.

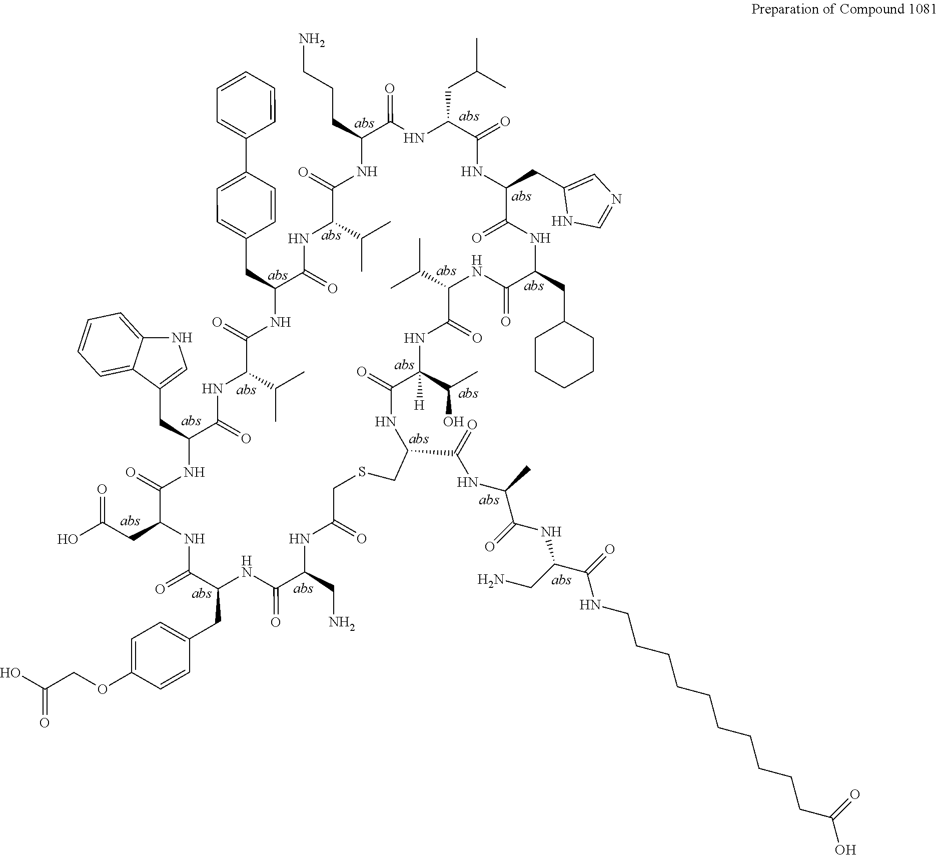 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1081 was prepared on a 50 μmol scale. The yield of the product was 28.9 mg, and its estimated purity by LCMS analysis was 84.6%. Analysis condition A: Retention time=1.81 min; ESI-MS(+) m/z [M+2H]²⁺: 1125.2.

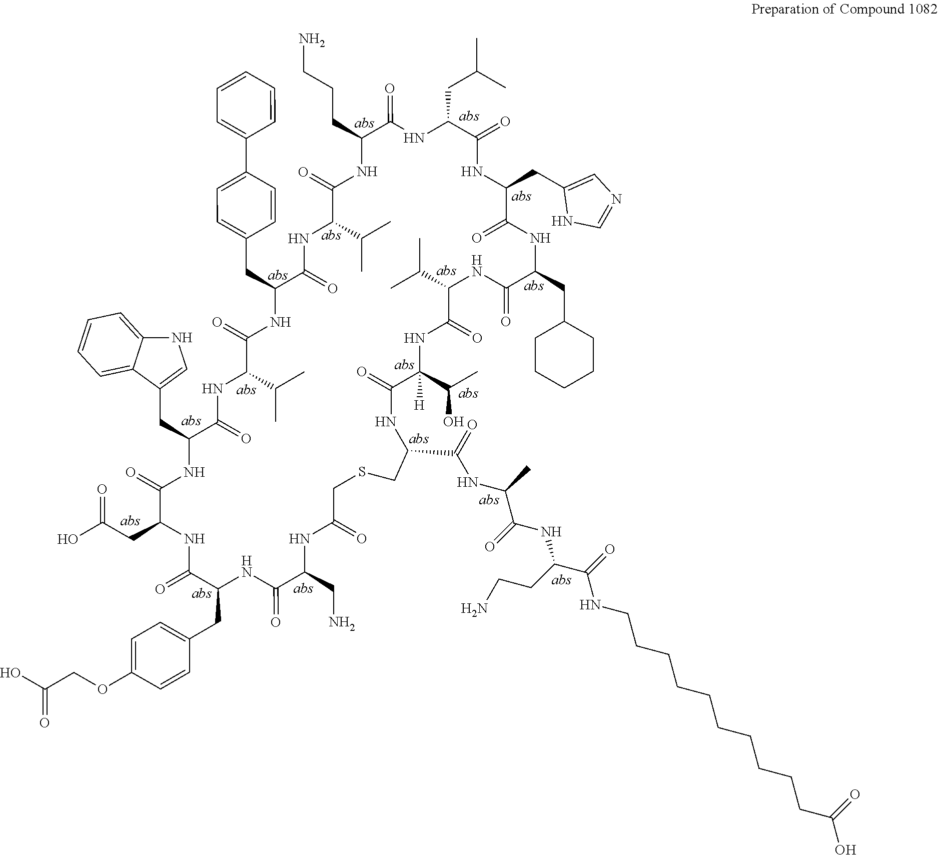 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1082 was prepared on a 50 μmol scale. The yield of the product was 9.1 mg, and its estimated purity by LCMS analysis was 90.2%. Analysis condition A: Retention time=1.7 min; ESI-MS(+) m/z [M+3H]³⁺: 755.1.

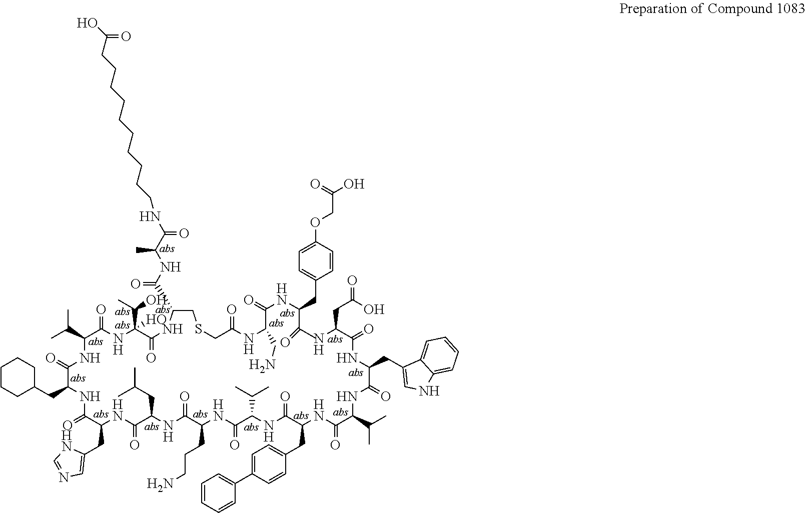 [SMILES: CC(=O)[C@H](Cc1ccc(OCC(=O)O)cc1)NC(=O)[C@H](CN)NC(=O)CSC[C@H](NC(=O)[C@@](O)(NC(=O)[C@@H](NC(=O)[C@H](CC1CCCCC1)NC(=O)[C@H](Cc1cnc[nH]1)NC(=O)[C@@H](CC(C)C)NC(=O)[C@H](CCCN)NC(=O)[C@@H](NC(=O)[C@H](Cc1ccc(-c2ccccc2)cc1)NC(=O)[C@@H](NC(=O)[C@H](Cc1c[nH]c2ccccc12)NC(=O)[C@@H](N)CC(=O)O)C(C)C)C(C)C)C(C)C)[C@@H](C)O)C(=O)N[C@@H](C)C(=O)NCCCCCCCCCCC(=O)O]

Compound 1083 was prepared on a 50 μmol scale. The yield of the product was 5 mg, and its estimated purity by LCMS analysis was 90.5%. Analysis condition B: Retention time=1.66 min; ESI-MS(+) m/z [M+3H]³⁺: 722.3.

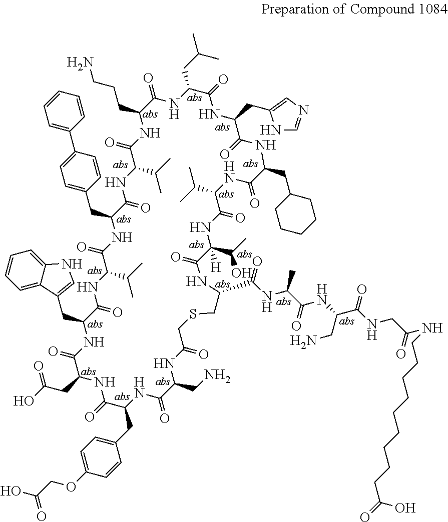 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CN)C(=O)NCC(=O)NCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1084 was prepared on a 50 μmol scale. The yield of the product was 3.7 mg, and its estimated purity by LCMS analysis was 95.4%. Analysis condition A: Retention time=1.69 min; ESI-MS(+) m/z [M+2H]²⁺: 1154.

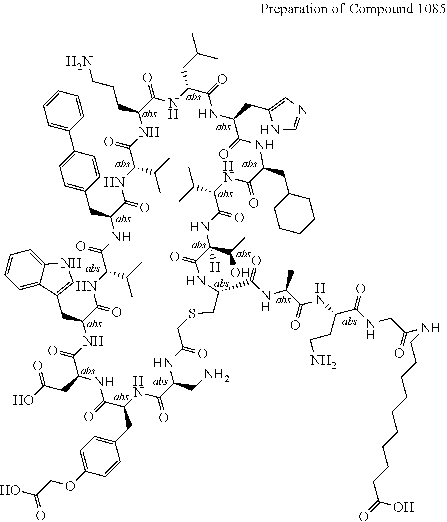 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCN)C(=O)NCC(=O)NCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1085 was prepared on a 50 μmol scale. The yield of the product was 19.9 mg, and its estimated purity by LCMS analysis was 85.7%. Analysis condition B: Retention time=1.52 min; ESI-MS(+) m/z [M+2H]²⁺: 1161.4.

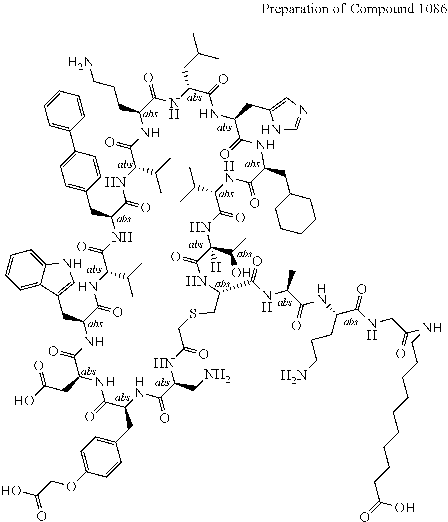 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCCN)C(=O)NCC(=O)NCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1086 was prepared on a 50 μmol scale. The yield of the product was 5 mg, and its estimated purity by LCMS analysis was 92.9%. Analysis condition A: Retention time=1.68 min; ESI-MS(+) m/z [M+2H]²⁺: 1167.9.

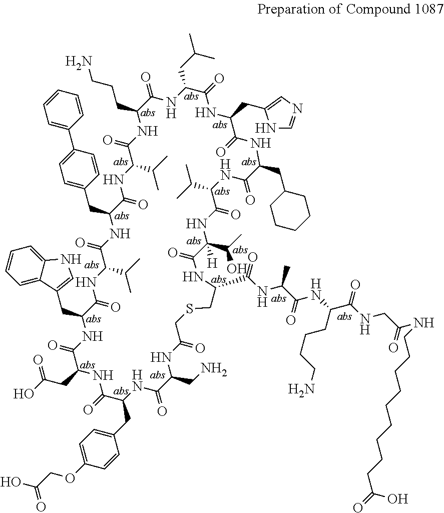 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCCCN)C(=O)NCC(=O)NCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1087 was prepared on a 50 μmol scale. The yield of the product was 21.6 mg, and its estimated purity by LCMS analysis was 85.9%. Analysis condition A: Retention time=1.68 min; ESI-MS(+) m/z [M+2H]²⁺:1175.

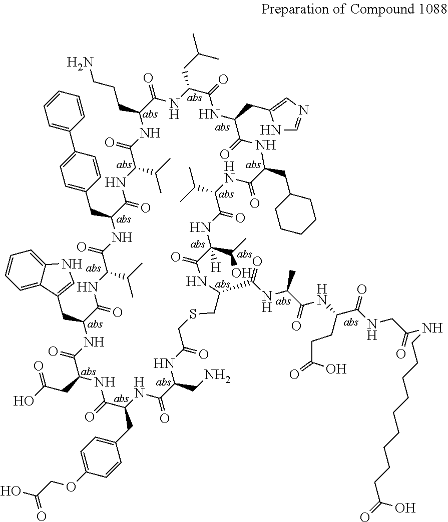 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCC(=O)O)C(=O)NCC(=O)NCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1088 was prepared on a 50 μmol scale. The yield of the product was 20.5 mg, and its estimated purity by LCMS analysis was 84%. Analysis condition A: Retention time=1.63 min; ESI-MS(+) m/z [M+2H]²⁺: 1176.

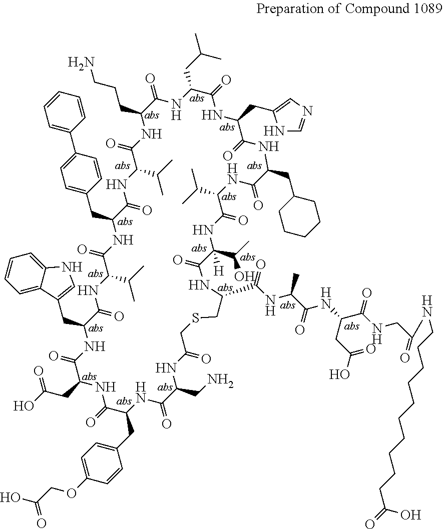 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CC(=O)O)C(=O)NCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1089 was prepared on a 50 μmol scale. The yield of the product was 7.4 mg, and its estimated purity by LCMS analysis was 94.1%. Analysis condition B: Retention time=1.56 min; ESI-MS(+) m/z [M+2H]²⁺:1168.2.

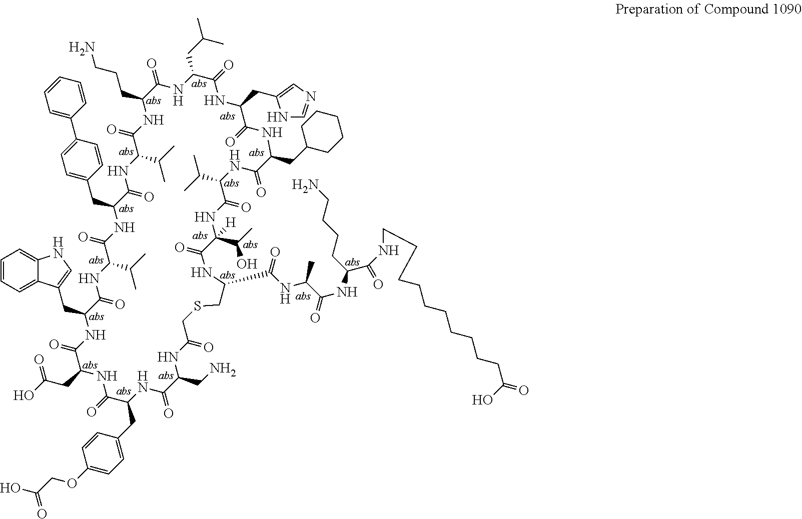 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1090 was prepared on a 50 μmol scale. The yield of the product was 7.6 mg, and its estimated purity by LCMS analysis was 90.1%. Analysis condition A: Retention time=1.69 min; ESI-MS(+) m/z [M+2H]²⁺: 1147.

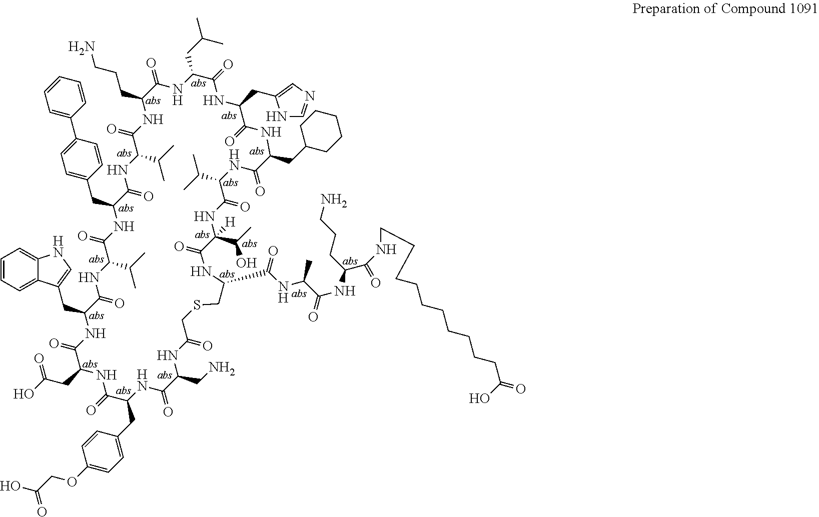 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1091 was prepared on a 50 μmol scale. The yield of the product was 18.3 mg, and its estimated purity by LCMS analysis was 93.5%. Analysis condition B: Retention time=1.51 min; ESI-MS(+) m/z [M+3H]³⁺: 760.1.

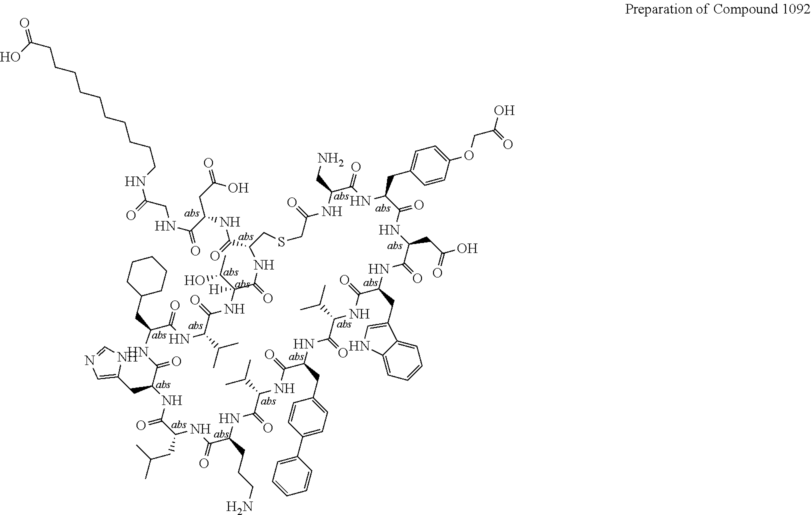 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CC(=O)O)C(=O)NCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1092 was prepared on a 50 μmol scale. The yield of the product was 2.6 mg, and its estimated purity by LCMS analysis was 90.4%. Analysis condition B: Retention time=1.6 min; ESI-MS(+) m/z [M+2H]²⁺: 1133.2.

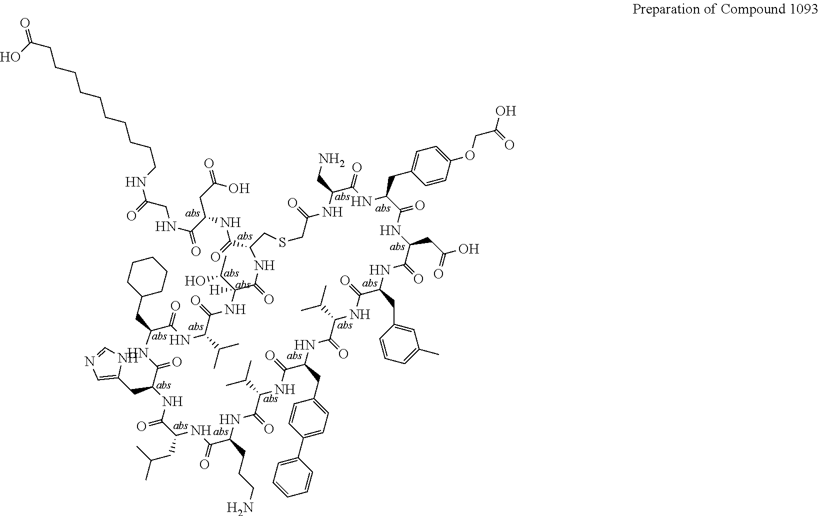 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)O)C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1093 was prepared on a 50 μmol scale. The yield of the product was 3.5 mg, and its estimated purity by LCMS analysis was 99%. Analysis condition A: Retention time=1.61 min; ESI-MS(+) m/z [M+2H]²⁺: 1121.

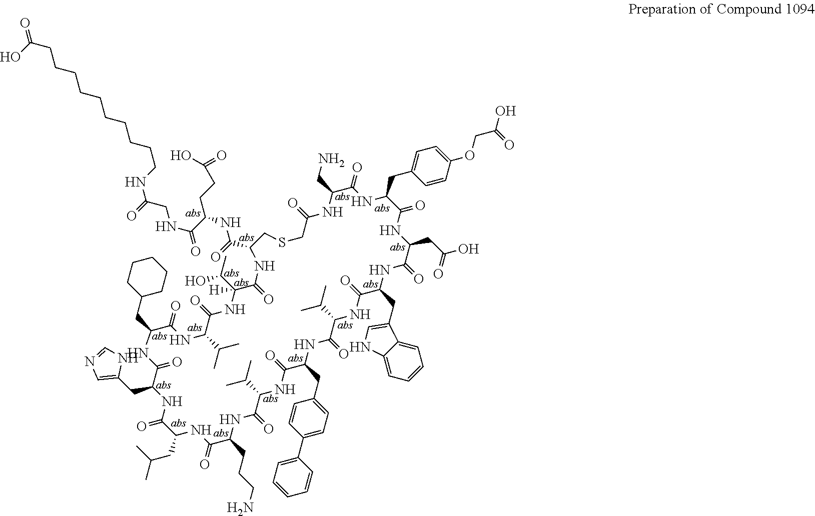 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCC(=O)O)C(=O)NCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1094 was prepared on a 50 μmol scale. The yield of the product was 9.2 mg, and its estimated purity by LCMS analysis was 94.6%. Analysis condition A: Retention time=1.62 min; ESI-MS(+) m/z [M+2H]²⁺: 1139.8.

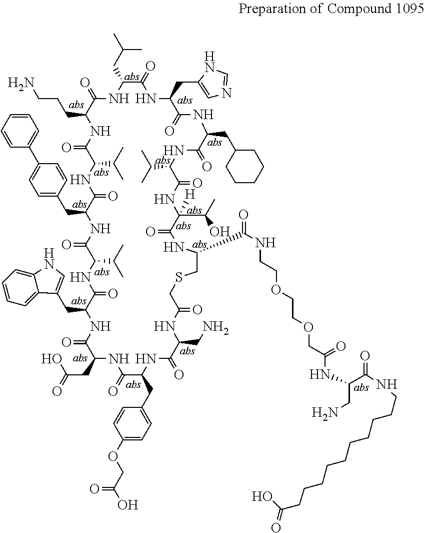 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1095 was prepared on a 50 μmol scale. The yield of the product was 46.3 mg, and its estimated purity by LCMS analysis was 93.6%. Analysis condition B: Retention time=1.57 min; ESI-MS(+) m/z [M+2H]²⁺: 1163.1.

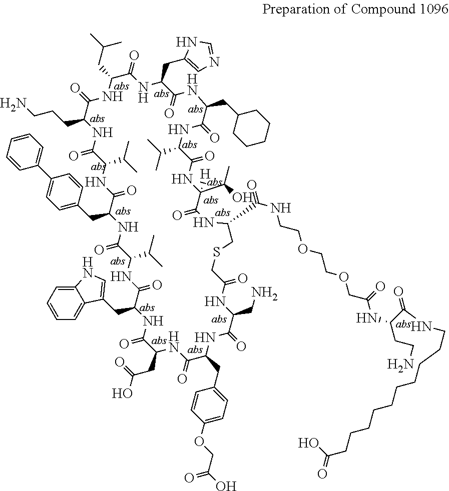 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1096 was prepared on a 50 μmol scale. The yield of the product was 25.1 mg, and its estimated purity by LCMS analysis was 100%. Analysis condition A: Retention time=1.64, 1.68 min; ESI-MS(+) m/z [M+2H]²⁺: 1169.27, 1169.27.

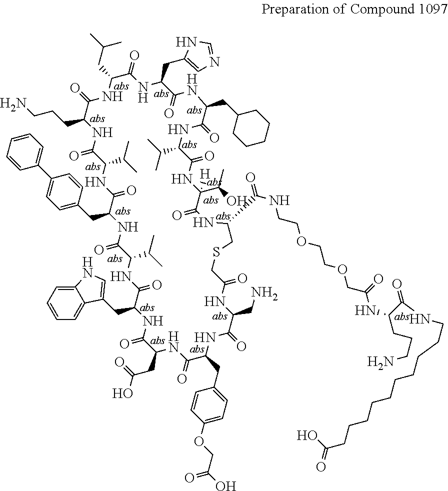 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1097 was prepared on a 50 μmol scale. The yield of the product was 15 mg, and its estimated purity by LCMS analysis was 98.8%. Analysis condition A: Retention time=1.67 min; ESI-MS(+) m/z [M+3H]³⁺: 785.

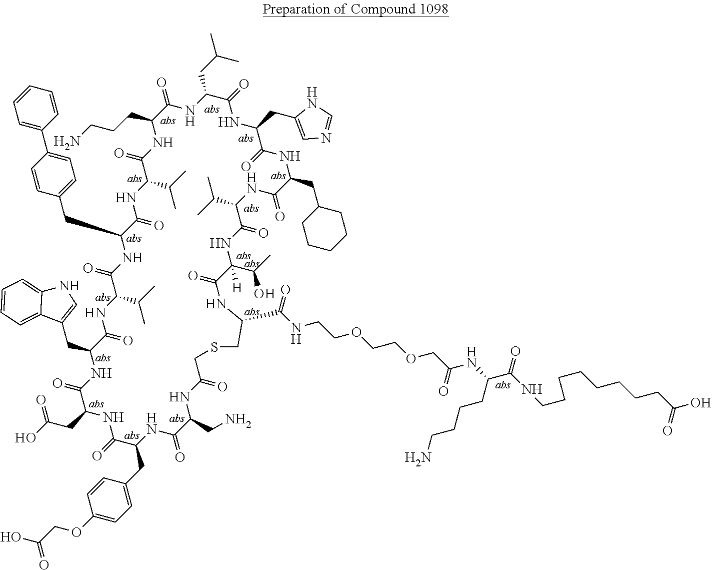 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCCN)C(=O)NCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1098 was prepared on a 50 μmol scale. The yield of the product was 34.2 mg, and its estimated purity by LCMS analysis was 91.9%. Analysis condition B: Retention time=1.51 min; ESI-MS(+) m/z [M+3H]³⁺: 789.1.

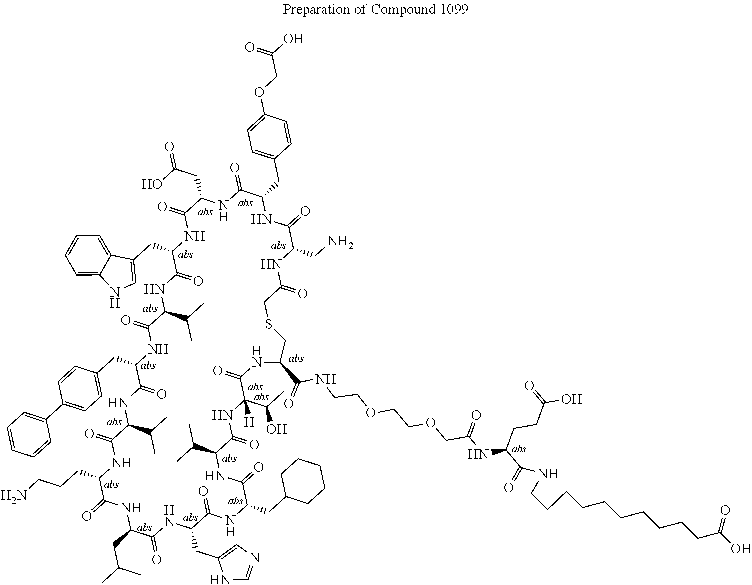 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1099 was prepared on a 50 μmol scale. The yield of the product was 46 mg, and its estimated purity by LCMS analysis was 91.3%. Analysis condition A: Retention time=1.65 min; ESI-MS(+) m/z [M+2H]²⁺: 1184.1.

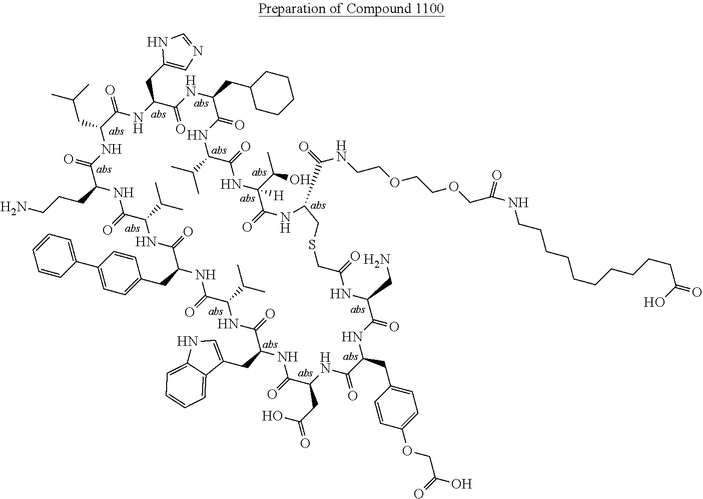 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1100 was prepared on a 50 μmol scale. The yield of the product was 25.1 mg, and its estimated purity by LCMS analysis was 100%. Analysis condition A: Retention time=1.78 min; ESI-MS(+) m/z [M+2H]²⁺: 1120.

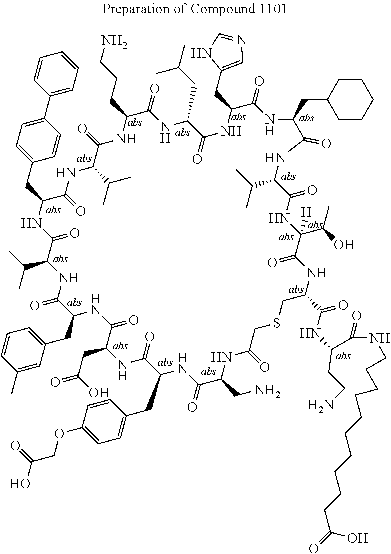 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1101 was prepared on a 50 μmol scale. The yield of the product was 9.5 mg, and its estimated purity by LCMS analysis was 92%. Analysis condition B: Retention time=1.6, 1.64 min; ESI-MS(+) m/z [M+2H]²⁺:1084.34, 1084.34.

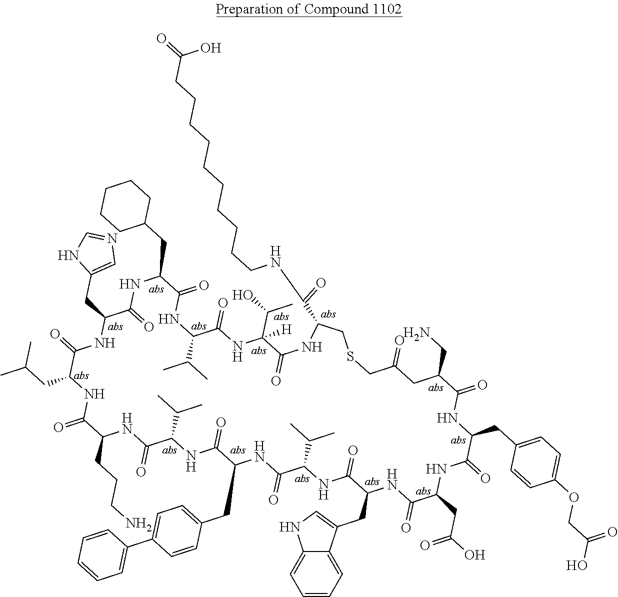 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)CC(=O)CSC[C@@H](C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1102 was prepared on a 50 μmol scale. The yield of the product was 9.8 mg, and its estimated purity by LCMS analysis was 86%. Analysis condition B: Retention time=1.7 min; ESI-MS(+) m/z [M+2H]²⁺: 1047.

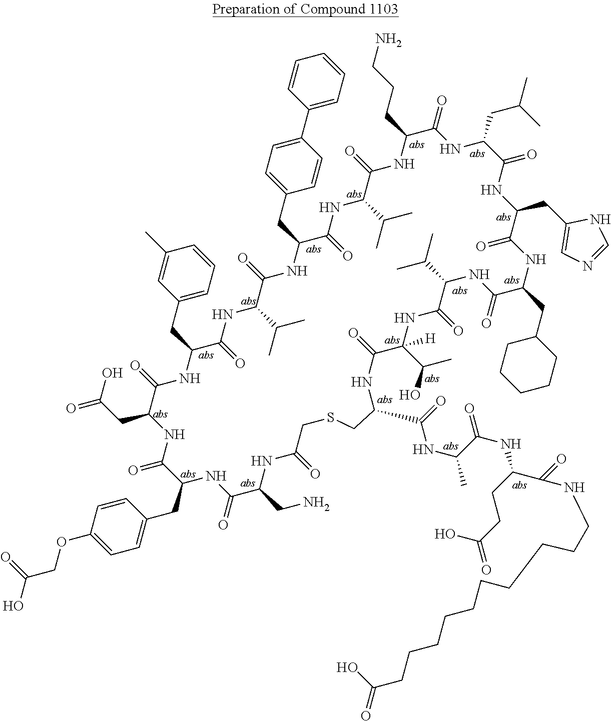 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1103 was prepared on a 50 μmol scale. The yield of the product was 4.4 mg, and its estimated purity by LCMS analysis was 96.8%. Analysis condition A: Retention time=1.74 min; ESI-MS(+) m/z [M+2H]²⁺: 1134.1.

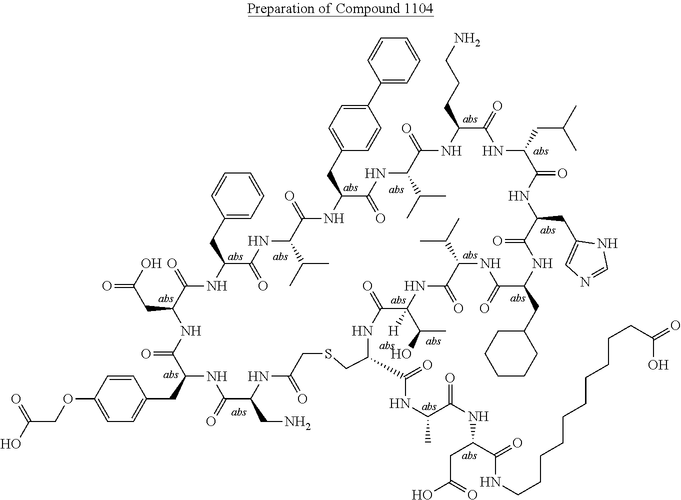 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccccc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CC(=O)O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1104 was prepared on a 50 μmol scale. The yield of the product was 1.3 mg, and its estimated purity by LCMS analysis was 95.2%. Analysis condition A: Retention time=1.58 min; ESI-MS(+) m/z [M+3H]³⁺: 752.2.

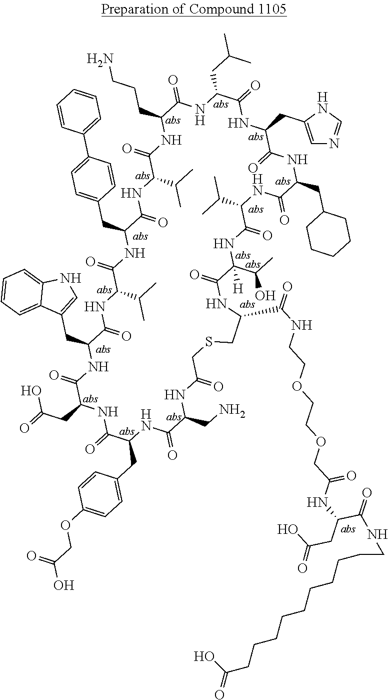 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CC(=O)O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1105 was prepared on a 50 μmol scale. The yield of the product was 39.5 mg, and its estimated purity by LCMS analysis was 98.9%. Analysis condition A: Retention time=1.6 min; ESI-MS(+) m/z [M+2H]²⁺: 1177.

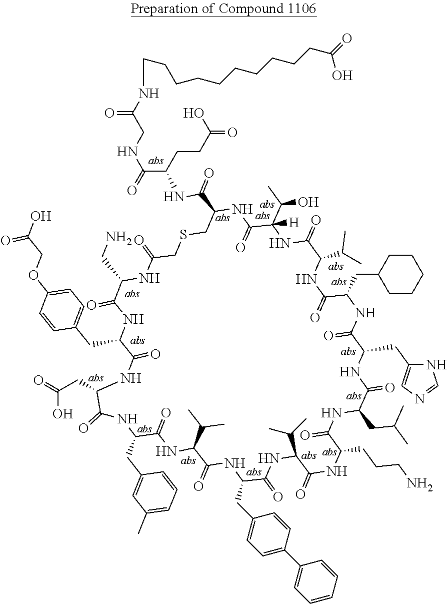 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCC(=O)O)C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1106 was prepared on a 50 μmol scale. The yield of the product was 16.6 mg, and its estimated purity by LCMS analysis was 98.5%. Analysis condition A: Retention time=1.62, 1.69 min; ESI-MS(+) m/z [M+2H]²⁺: 1128.

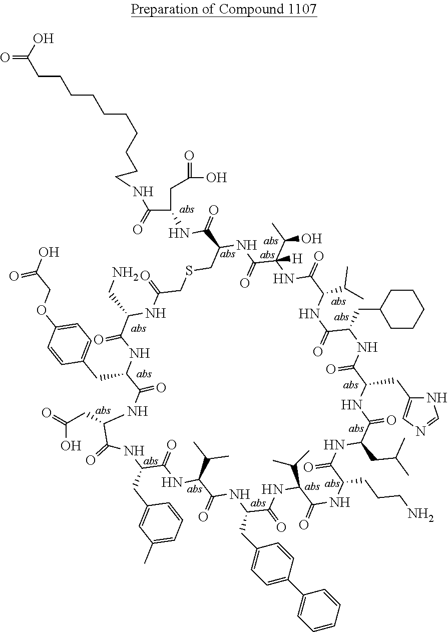 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1107 was prepared on a 50 μmol scale. The yield of the product was 5.3 mg, and its estimated purity by LCMS analysis was 95.1%. Analysis condition A: Retention time=1.65 min; ESI-MS(+) m/z [M+2H]²⁺: 1091.9.

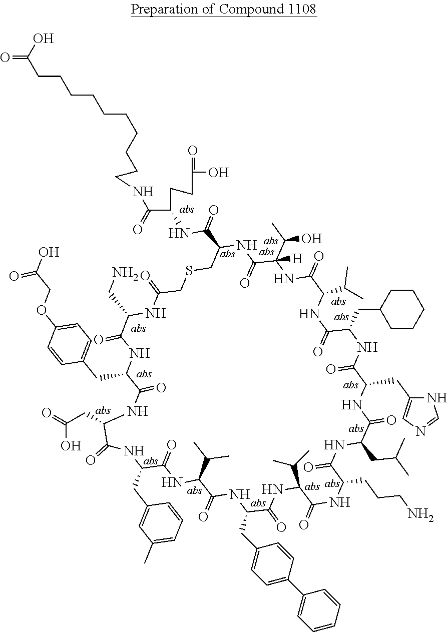 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1108 was prepared on a 50 μmol scale. The yield of the product was 4.6 mg, and its estimated purity by LCMS analysis was 91.1%. Analysis condition B: Retention time=1.67 min; ESI-MS(+) m/z [M+2H]²⁺: 1099.4.

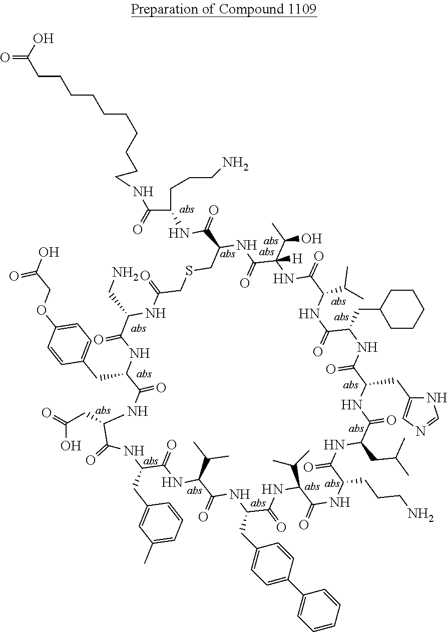 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1109 was prepared on a 50 μmol scale. The yield of the product was 11.3 mg, and its estimated purity by LCMS analysis was 85.5%. Analysis condition B: Retention time=1.58 min; ESI-MS(+) m/z [M+3H]³⁺: 728.1.

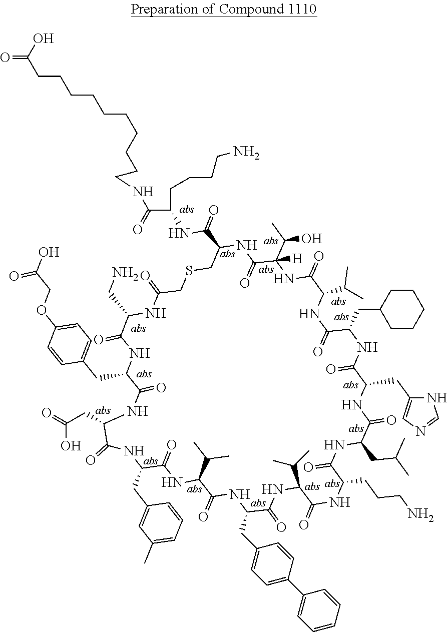 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1110 was prepared on a 50 μmol scale. The yield of the product was 2.6 mg, and its estimated purity by LCMS analysis was 100%. Analysis condition B: Retention time=1.59 min; ESI-MS(+) m/z [M+2H]²⁺: 1098.3.

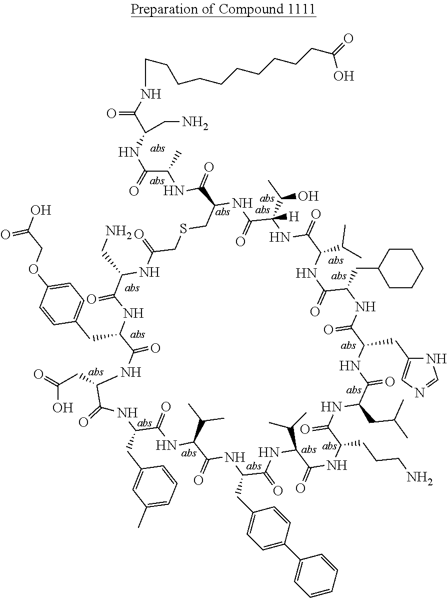 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1111 was prepared on a 50 μmol scale. The yield of the product was 6.8 mg, and its estimated purity by LCMS analysis was 100%. Analysis condition A: Retention time=1.6 min; ESI-MS(+) m/z [M+3H]³⁺: 742.5.

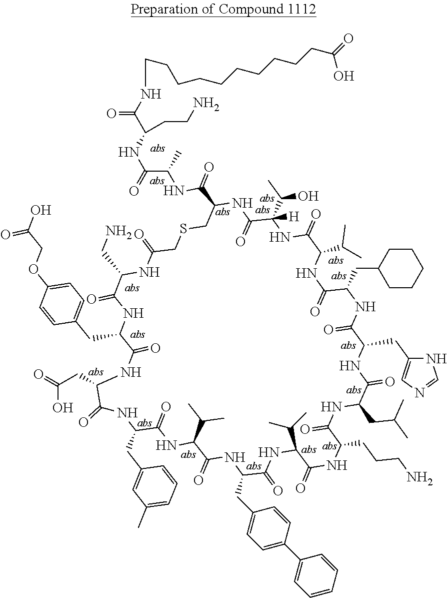 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1112 was prepared on a 50 μmol scale. The yield of the product was 7.3 mg, and its estimated purity by LCMS analysis was 100%. Analysis condition A: Retention time=1.62 min; ESI-MS(+) m/z [M+2H]²⁺: 1120.2.

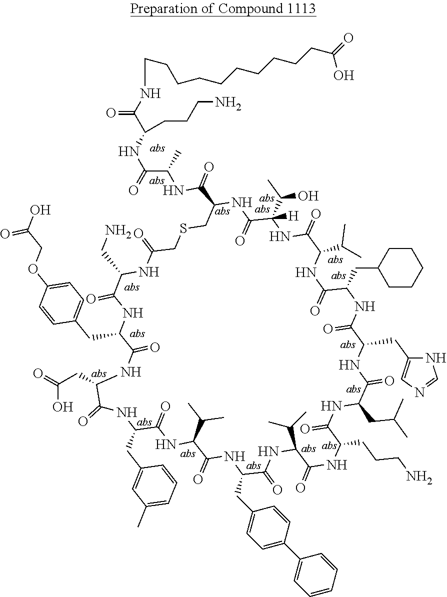 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1113 was prepared on a 50 μmol scale. The yield of the product was 8 mg, and its estimated purity by LCMS analysis was 100%. Analysis condition A: Retention time=1.76 min; ESI-MS(+) m/z [M+2H]²⁺: 1126.9.

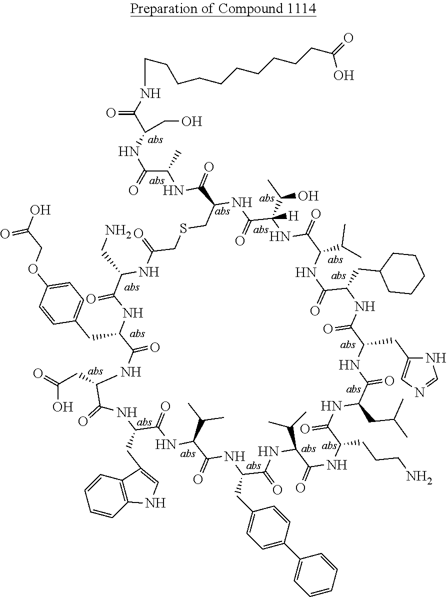 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](CO)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1114 was prepared on a 50 μmol scale. The yield of the product was 1.6 mg, and its estimated purity by LCMS analysis was 95.5%. Analysis condition A: Retention time=1.67, 1.73 min; ESI-MS(+) m/z [M+2H]²⁺: 1127.

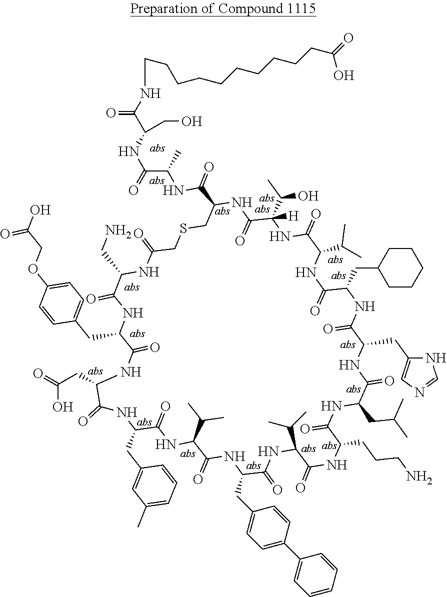 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CO)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1115 was prepared on a 50 μmol scale. The yield of the product was 2.6 mg, and its estimated purity by LCMS analysis was 95.1%. Analysis condition A: Retention time=1.62, 1.65, 1.69 min; ESI-MS(+) m/z [M+2H]²⁺: 1114.

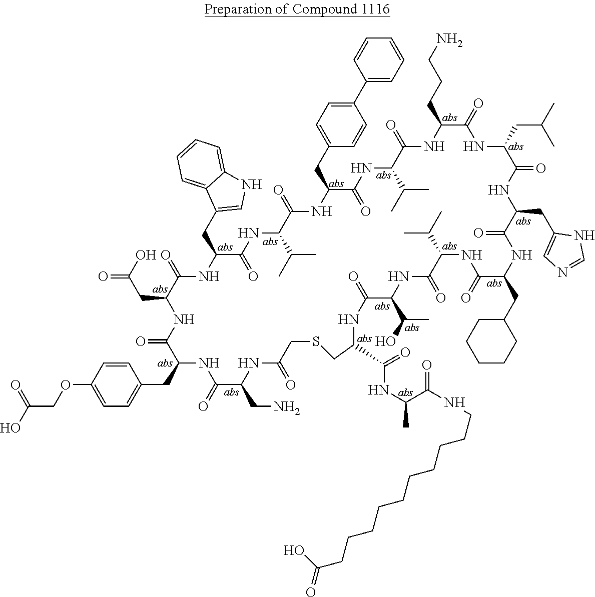

Compound 1116 was prepared on a 50 μmol scale. The yield of the product was 20.1 mg, and its estimated purity by LCMS analysis was 96.7%. Analysis condition A: Retention time=1.78 min; ESI-MS(+) m/z [M+3H]³⁺: 722.1.

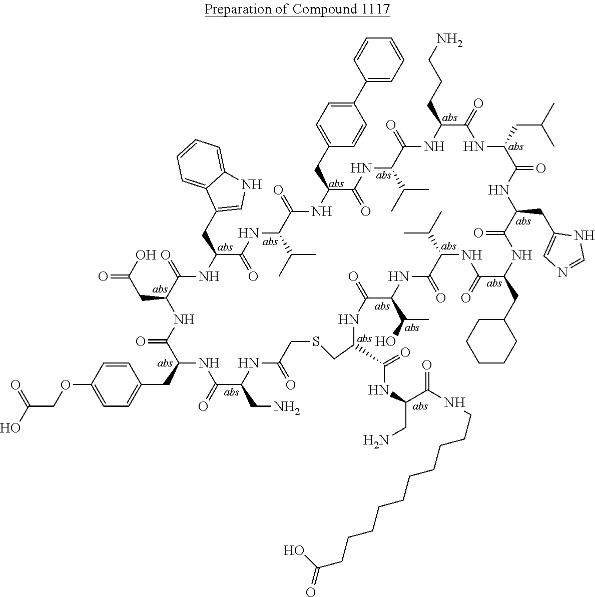

Compound 1117 was prepared on a 50 μmol scale. The yield of the product was 34 mg, and its estimated purity by LCMS analysis was 98.6%. Analysis condition A: Retention time=1.76 min; ESI-MS(+) m/z [M+2H]²⁺: 1090.

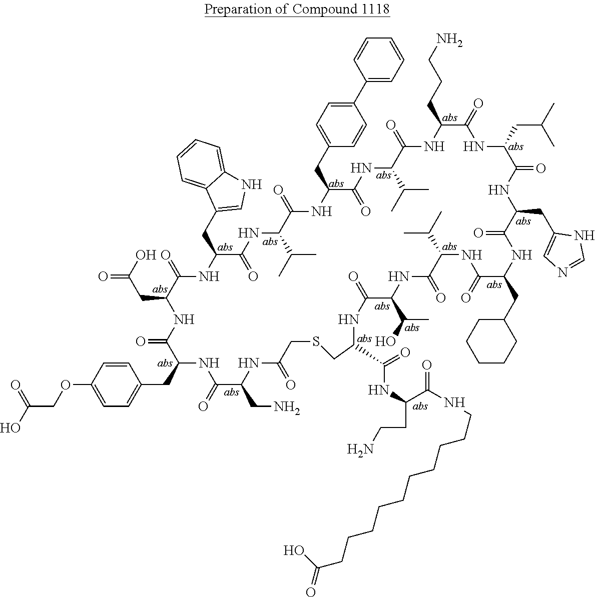

Compound 1118 was prepared on a 50 μmol scale. The yield of the product was 10.8 mg, and its estimated purity by LCMS analysis was 82.5%. Analysis condition B: Retention time=1.57 min; ESI-MS(+) m/z [M+2H]²⁺: 1097.1.

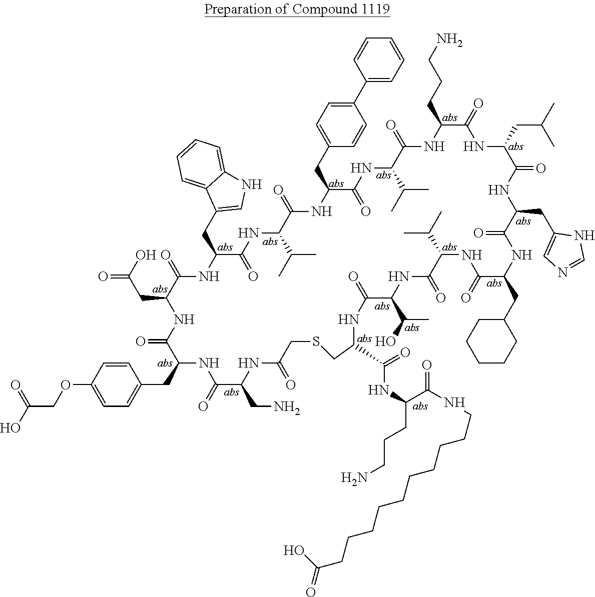

Compound 1119 was prepared on a 50 μmol scale. The yield of the product was 30.1 mg, and its estimated purity by LCMS analysis was 85.3%. Analysis condition A: Retention time=1.75 min; ESI-MS(+) m/z [M+2H]²⁺: 1104.1.

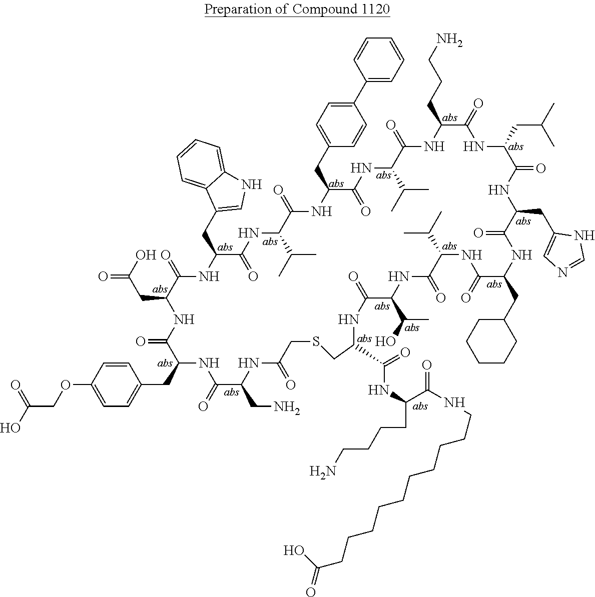

Compound 1120 was prepared on a 50 μmol scale. The yield of the product was 6.8 mg, and its estimated purity by LCMS analysis was 86.2%. Analysis condition A: Retention time=1.75 min; ESI-MS(+) m/z [M+2H]²⁺: 1110.9.

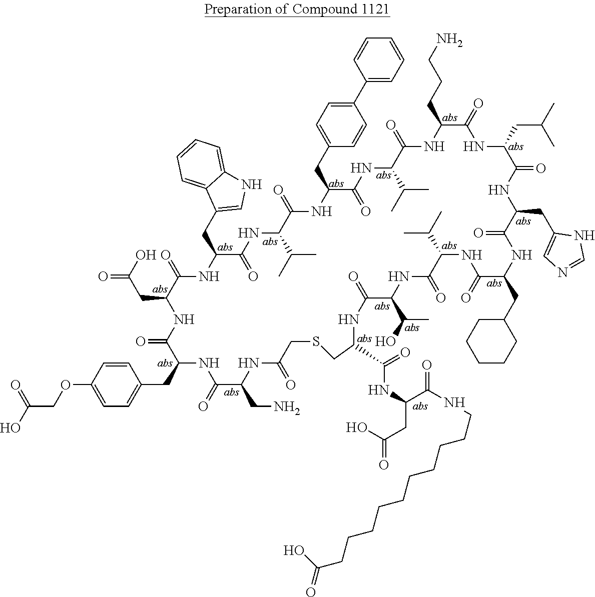

Compound 1121 was prepared on a 50 μmol scale. The yield of the product was 16.4 mg, and its estimated purity by LCMS analysis was 88%. Analysis condition A: Retention time=1.75 min; ESI-MS(+) m/z [M+2H]²⁺: 1104.2.

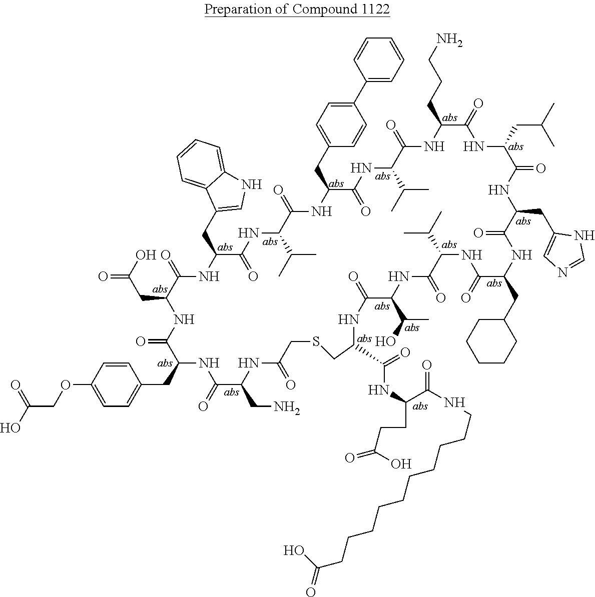

Compound 1122 was prepared on a 50 μmol scale. The yield of the product was 3.9 mg, and its estimated purity by LCMS analysis was 100%. Analysis condition B: Retention time=1.67 min; ESI-MS(+) m/z [M+2H]²⁺: 1111.2.

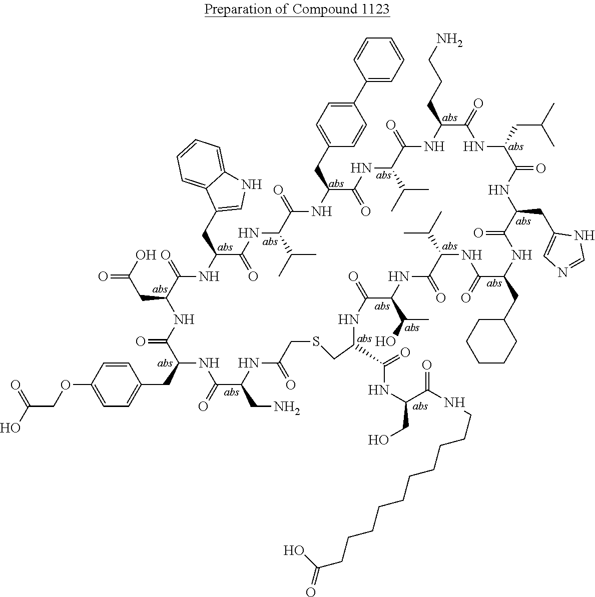

Compound 1123 was prepared on a 50 μmol scale. The yield of the product was 6.8 mg, and its estimated purity by LCMS analysis was 100%. Analysis condition B: Retention time=1.66 min; ESI-MS(+) m/z [M+3H]³⁺: 727.4.

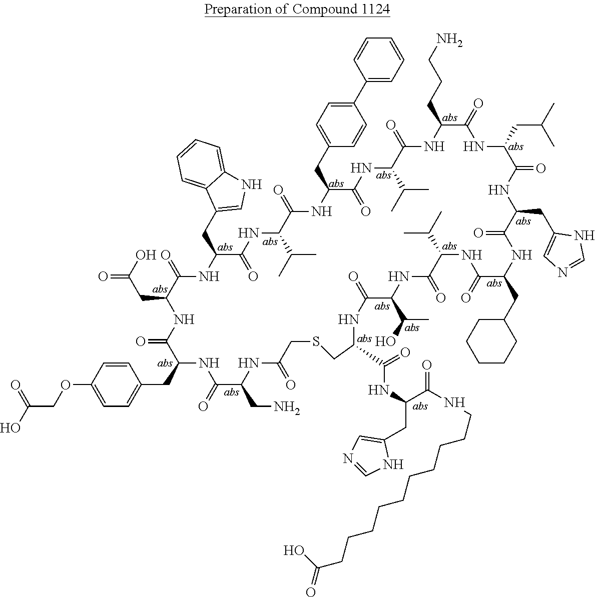 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@H](Cc2cnc[nH]2)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1124 was prepared on a 50 μmol scale. The yield of the product was 14.3 mg, and its estimated purity by LCMS analysis was 100%. Analysis condition A: Retention time=1.61 min; ESI-MS(+) m/z [M+3H]³⁺: 744.3.

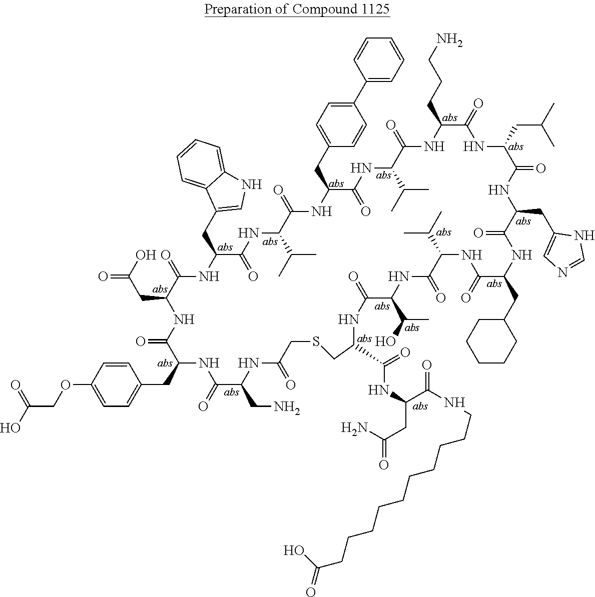

Compound 1125 was prepared on a 50 μmol scale. The yield of the product was 8.1 mg, and its estimated purity by LCMS analysis was 95%. Analysis condition A: Retention time=1.79 min; ESI-MS(+) m/z [M+2H]²⁺: 1103.8.

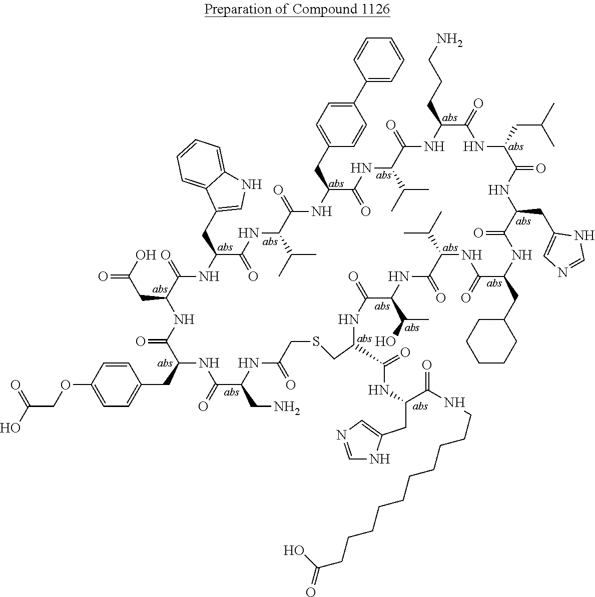

Compound 1126 was prepared on a 50 μmol scale. The yield of the product was 6.6 mg, and its estimated purity by LCMS analysis was 100%. Analysis condition A: Retention time=1.74 min; ESI-MS(+) m/z [M+3H]³⁺: 744.2.

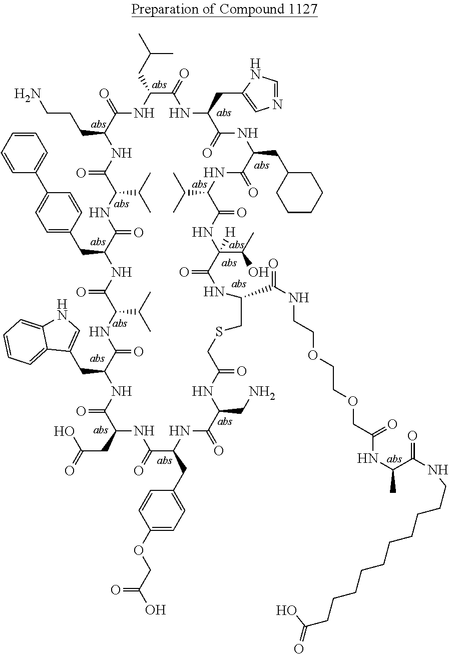 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](C)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1127 was prepared on a 50 μmol scale. The yield of the product was 10 mg, and its estimated purity by LCMS analysis was 90.6%. Analysis condition B: Retention time=1.54 min; ESI-MS(+) m/z [M+2H]²⁺: 1155.

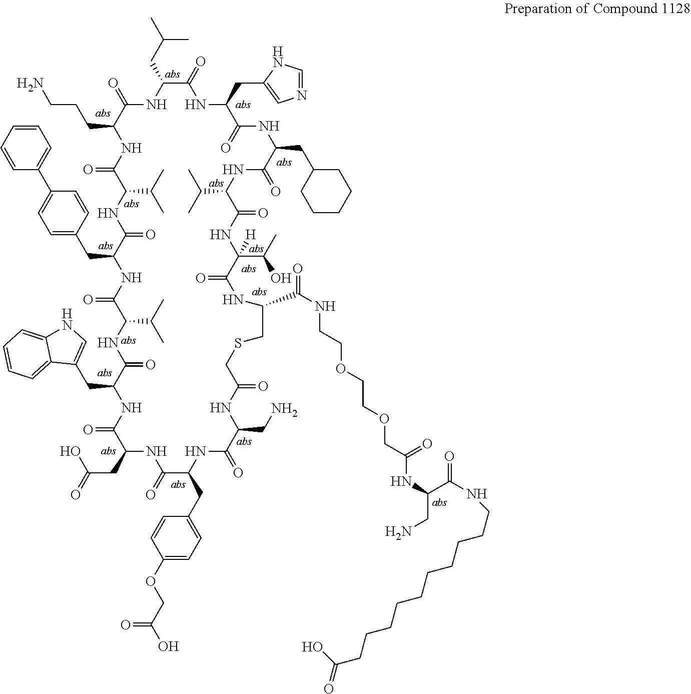 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](CN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1128 was prepared on a 50 μmol scale. I ne yield of the product was 14.9 mg, and its estimated purity by LCMS analysis was 85.7%. Analysis condition B: Retention time=1.55 min; ESI-MS(+) m/z [M+2H]²⁺: 1162.2.

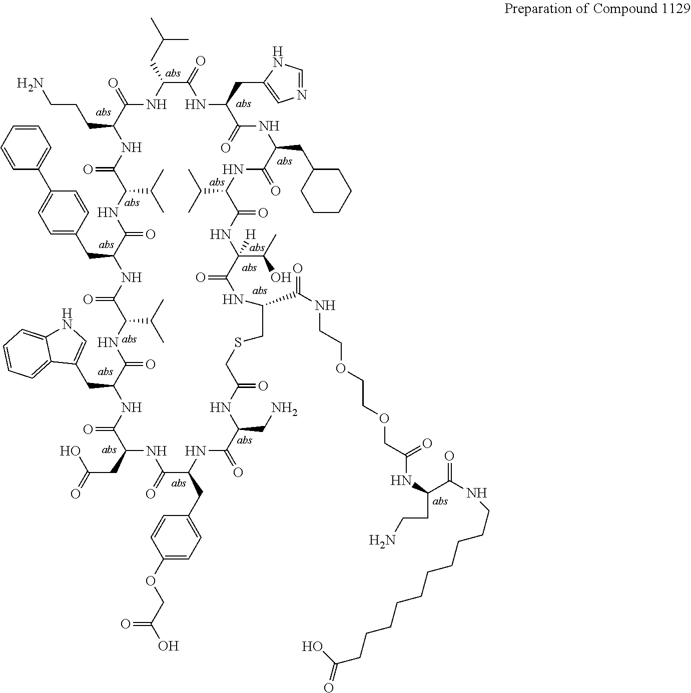 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1129 was prepared on a 50 μmol scale. The yield of the product was 3.9 mg, and its estimated purity by LCMS analysis was 100%. Analysis condition A: Retention time=1.67, 1.7 min; ESI-MS(+) m/z [M+2H]²⁺: 1169.23, 1169.23.

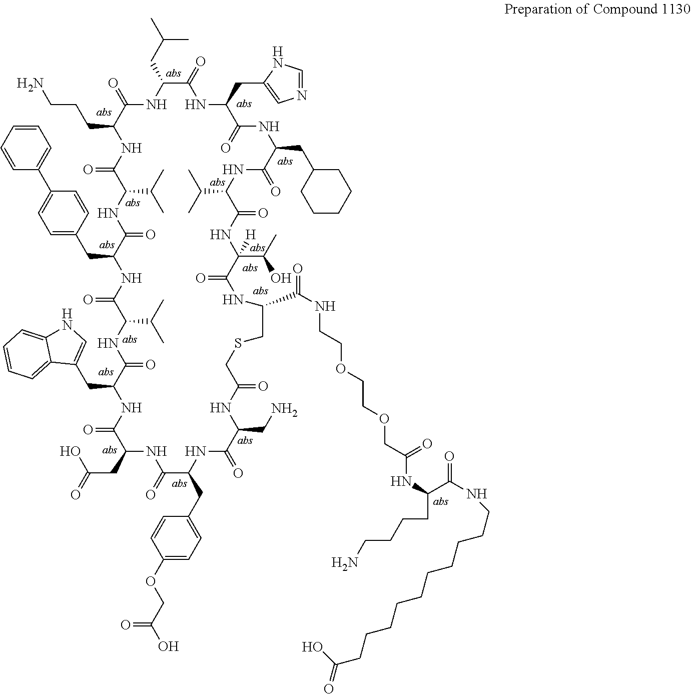 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](CCCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1130 was prepared on a 50 μmol scale. The yield of the product was 21 mg, and its estimated purity by LCMS analysis was 91.6%. Analysis condition A: Retention time=2.45 min; ESI-MS(+) m/z [M+3H]³⁺: 785.

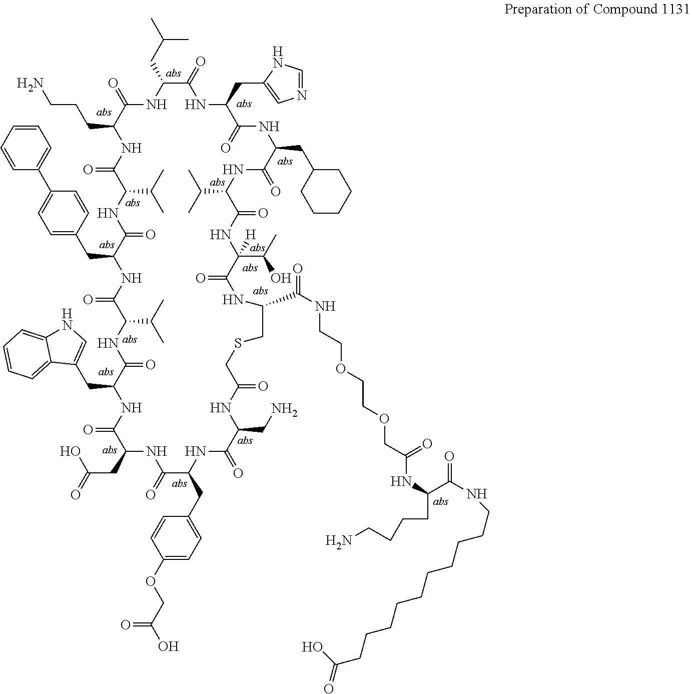 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](CCCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1131 was prepared on a 50 μmol scale. The yield of the product was 35 mg, and its estimated purity by LCMS analysis was 98.9%. Analysis condition A: Retention time=1.68, 1.72 min; ESI-MS(+) m/z [M+3H]³⁺: 789.27, 789.27.

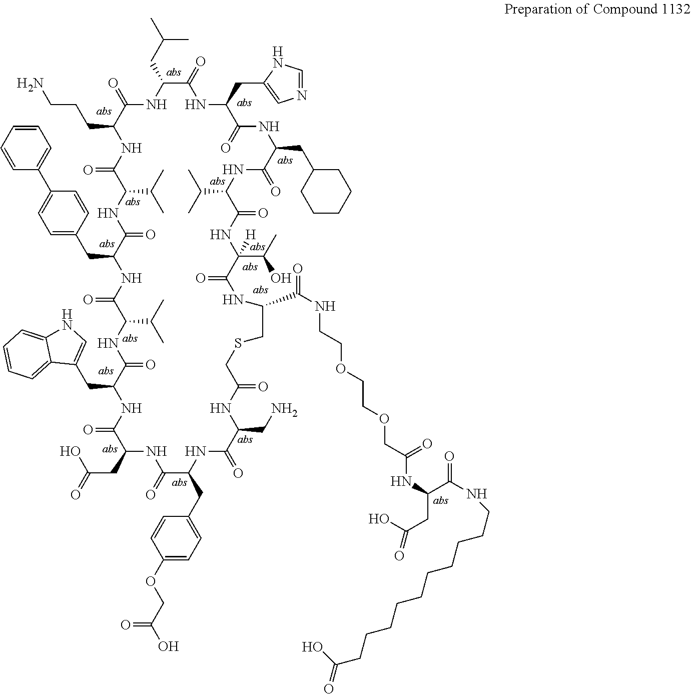 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](CC(=O)O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1132 was prepared on a 50 μmol scale. The yield of the product was 23.2 mg, and its estimated purity by LCMS analysis was 100%. Analysis condition A: Retention time=1.5, 1.54 min; ESI-MS(+) m/z [M+2H]²⁺:1177.27, 1177.08.

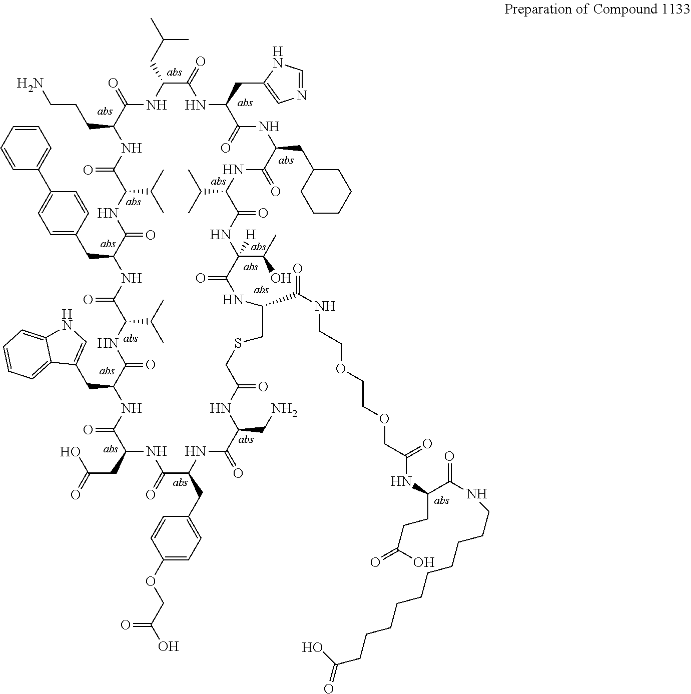 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](CCC(=O)O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1133 was prepared on a 50 μmol scale. The yield of the product was 37.1 mg, and its estimated purity by LCMS analysis was 100%. Analysis condition A: Retention time=1.62, 1.66 min; ESI-MS(+) m/z [M+2H]²⁺: 1184.

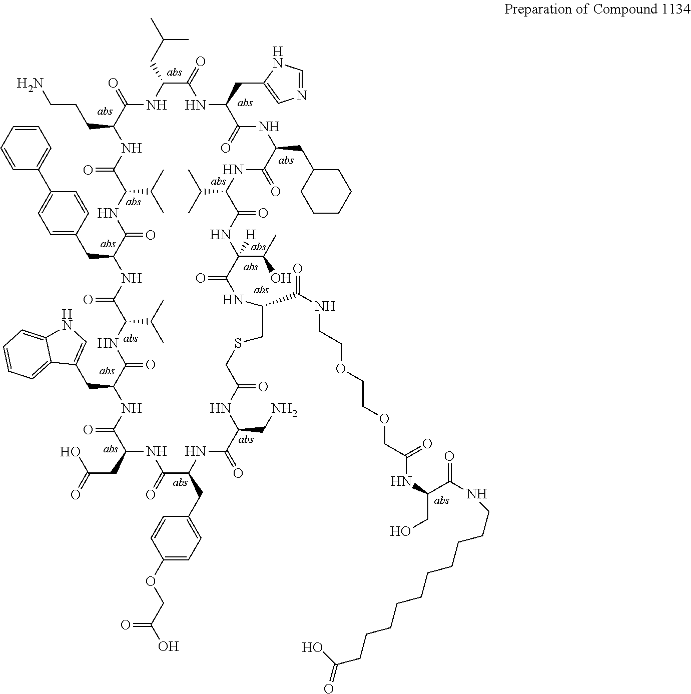 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](CO)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1134 was prepared on a 50 μmol scale. The yield of the product was 42.7 mg, and its estimated purity by LCMS analysis was 98.5%. Analysis condition A: Retention time=1.73, 1.76 min; ESI-MS(+) m/z [M+2H]²⁺: 1163.12, 1163.12.

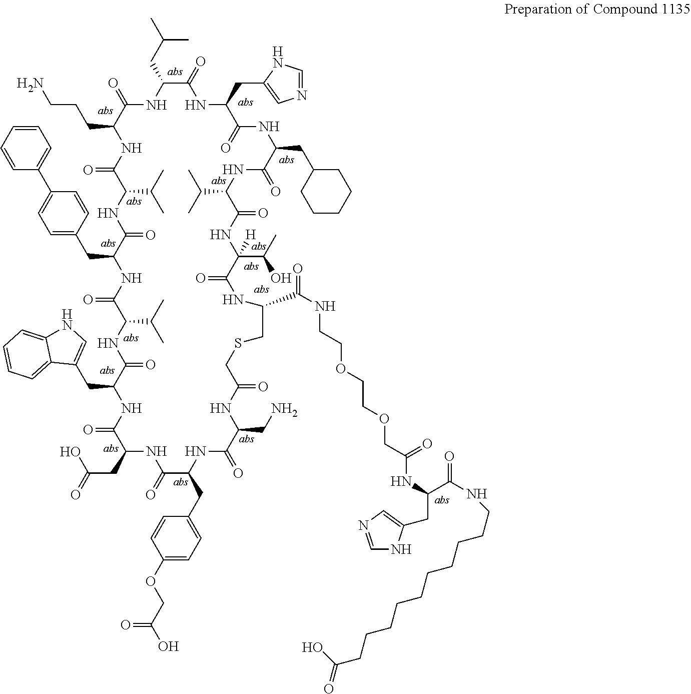 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](Cc2cnc[nH]2)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1135 was prepared on a 50 μmol scale. The yield of the product was 27.5 mg, and its estimated purity by LCMS analysis was 85.5%. Analysis condition B: Retention time=1.43 min; ESI-MS(+) m/z [M+3H]³⁺: 792.2.

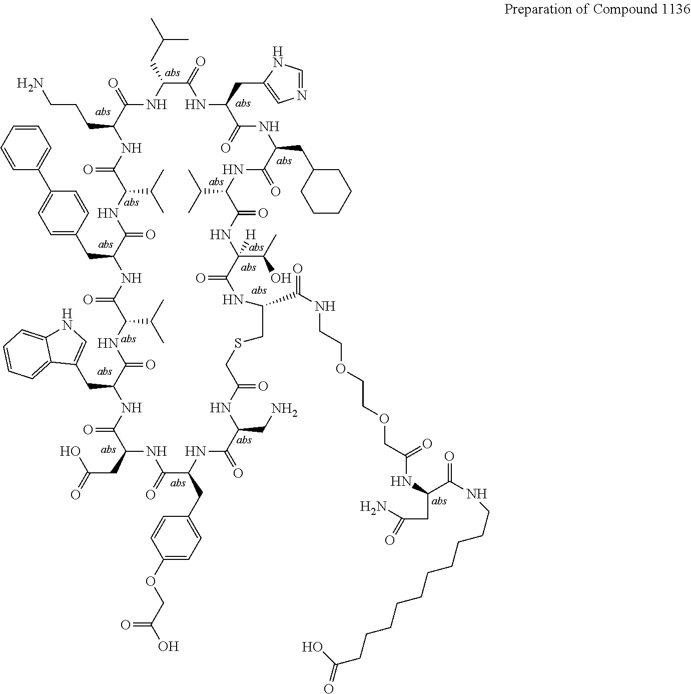 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@H](CC(N)=O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1136 was prepared on a 50 μmol scale. The yield of the product was 15.9 mg, and its estimated purity by LCMS analysis was 85.4%. Analysis condition A: Retention time=1.59 min; ESI-MS(+) m/z [M+3H]³⁺: 784.4.

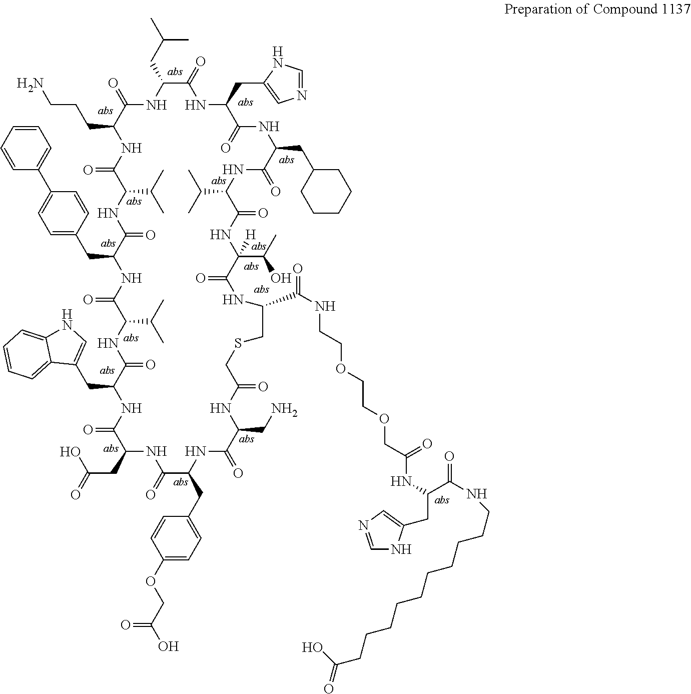

Compound 1137 was prepared on a 50 μmol scale. The yield of the product was 30.4 mg, and its estimated purity by LCMS analysis was 98.7%. Analysis condition A: Retention time=1.68, 1.72 min; ESI-MS(+) m/z [M+2H]²⁺: 1188.

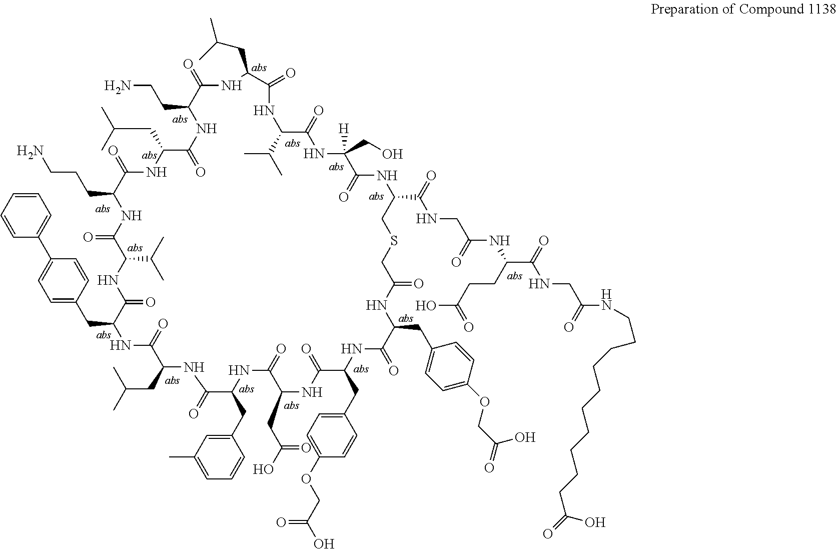 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(=O)N[C@@H](CCC(=O)O)C(=O)NCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1138 was prepared on a 50 μmol scale. The yield of the product was 3.1 mg, and its estimated purity by LCMS analysis was 92.8%. Analysis condition B: Retention time=2.42 min; ESI-MS(+) m/z [M+2H]²⁺: 1185.

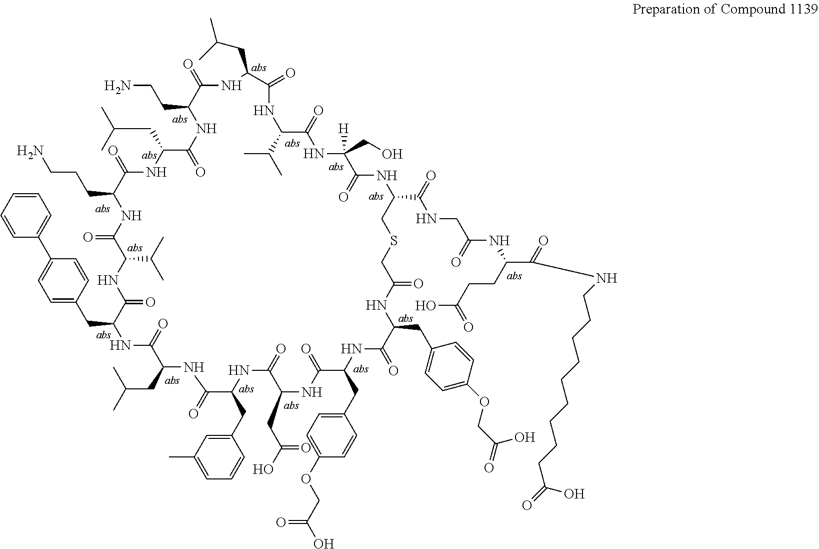 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(=O)N[C@@H](CCC(=O)O)C(=O)NCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1139 was prepared on a 50 μmol scale. The yield of the product was 8.2 mg, and its estimated purity by LCMS analysis was 80.5%. Analysis condition B: Retention time=1.63 min; ESI-MS(+) m/z [M+3H]³⁺: 771.1.

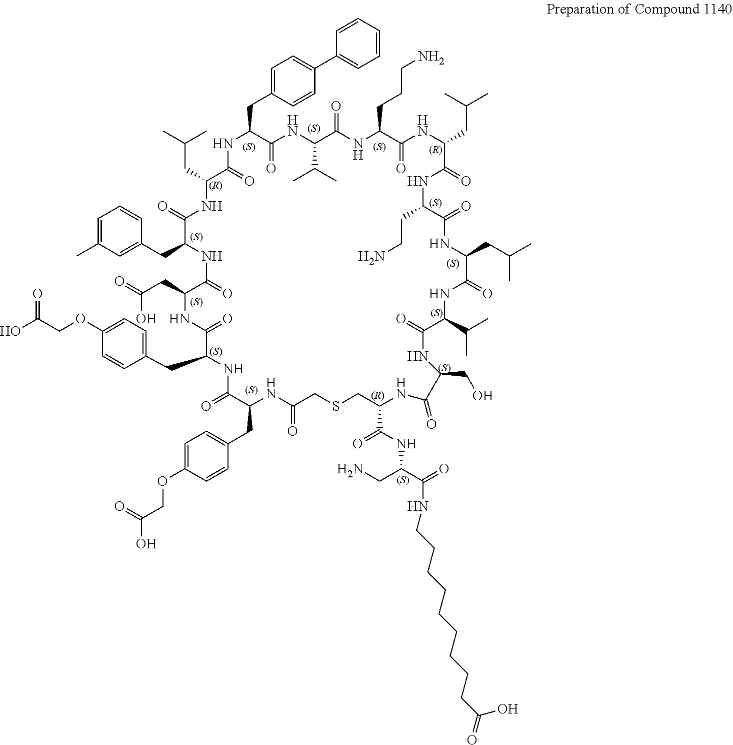

Compound 1140 was prepared on a 50 μmol scale. The yield of the product was 1.3 mg, and its estimated purity by LCMS analysis was 80.1%. Analysis condition B: Retention time=1.71 min; ESI-MS(+) m/z [M+2H]²⁺: 1106.2.

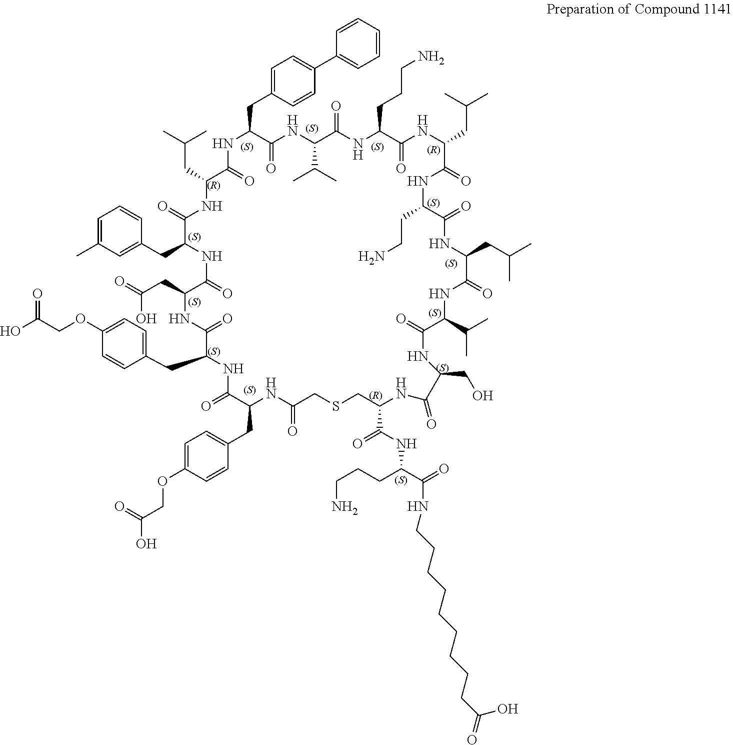

Compound 1141 was prepared on a 50 μmol scale. The yield of the product was 1.7 mg, and its estimated purity by LCMS analysis was 76.4%. Analysis condition B: Retention time=1.65 min; ESI-MS(+) m/z [M+2H]²⁺: 1120.5.

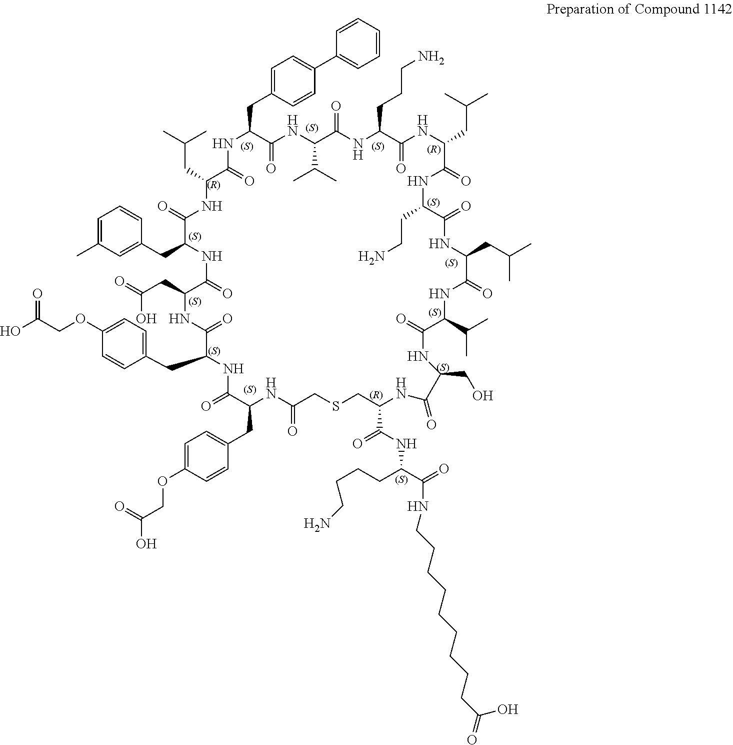

Compound 1142 was prepared on a 50 μmol scale. The yield of the product was 1.4 mg, and its estimated purity by LCMS analysis was 88.5%. Analysis condition B: Retention time=1.62, 1.65 min; ESI-MS(+) m/z [M+3H]³⁺: 752.1.

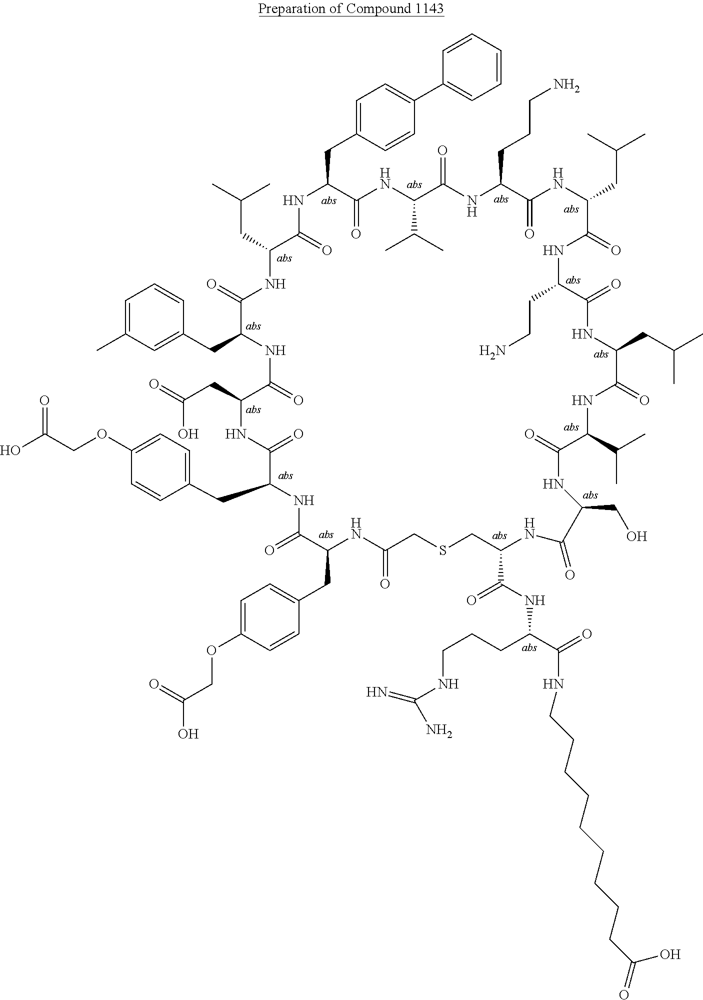 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCCNC(=N)N)C(=O)NCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@@H](CC(C)C)NC2=O)c1]

Compound 1143 was prepared on a 50 μmol scale. The yield of the product was 1.7 mg, and its estimated purity by LCMS analysis was 86.8%. Analysis condition A: Retention time=1.74 min; ESI-MS(+) m/z [M+2H]²⁺: 1141.2.

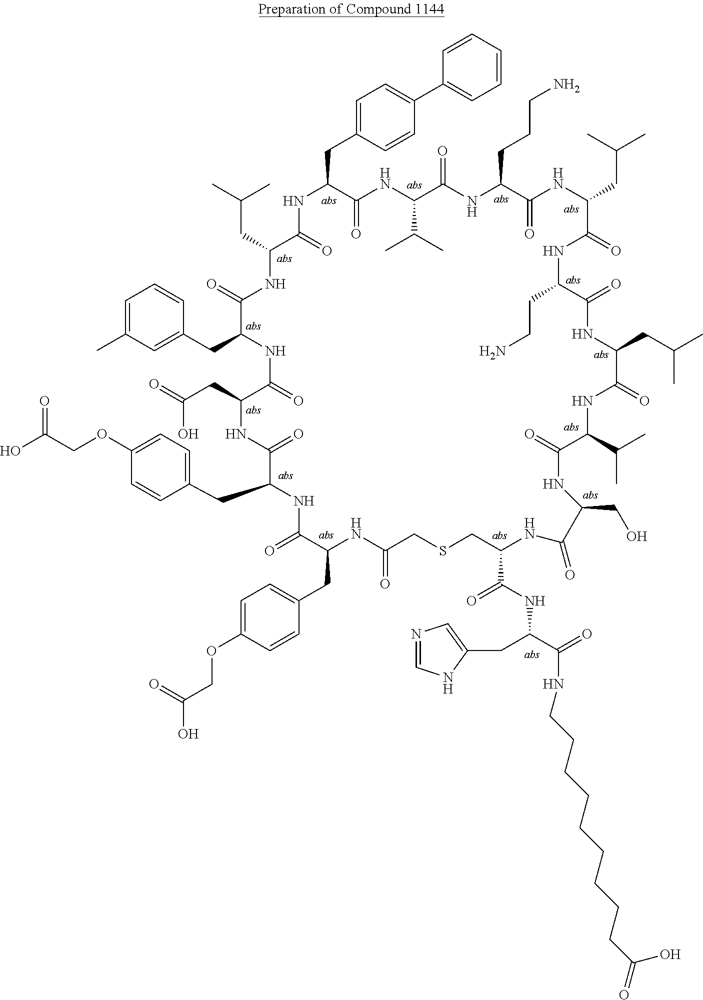 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](Cc3cnc[nH]3)C(=O)NCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@@H](CC(C)C)NC2=O)c1]

Compound 1144 was prepared on a 50 μmol scale. The yield of the product was 1.3 mg, and its estimated purity by LCMS analysis was 85.9%. Analysis condition B: Retention time=1.77 min; ESI-MS(+) m/z [M+2H]²⁺: 1132.1.

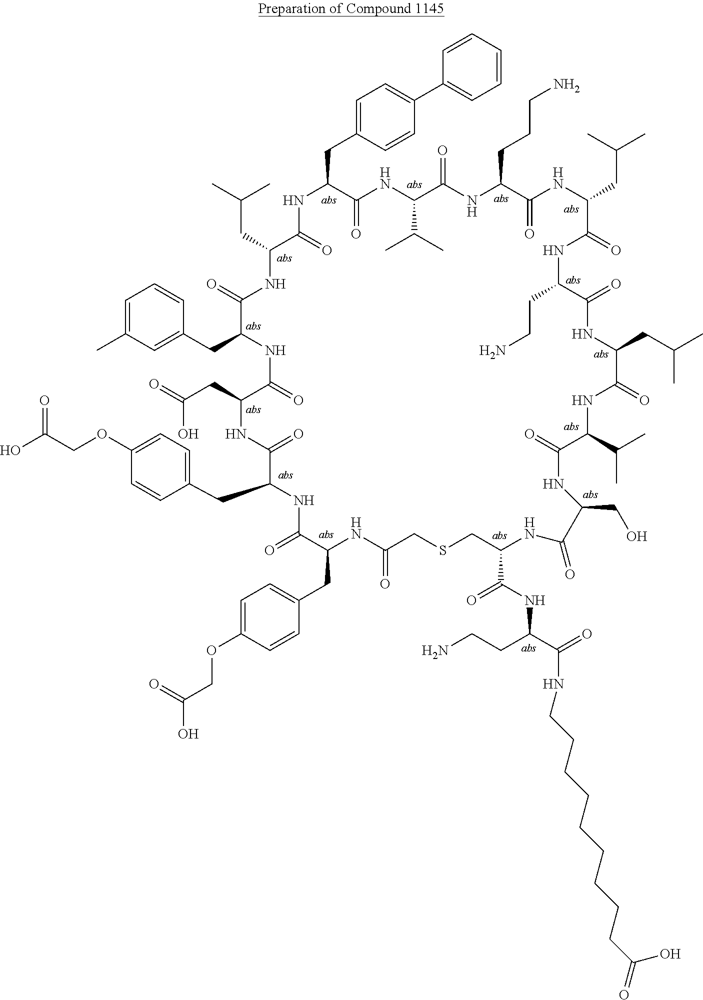 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@H](CCN)C(=O)NCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@@H](CC(C)C)NC2=O)c1]

Compound 1145 was prepared on a 50 μmol scale. The yield of the product was 3.7 mg, and its estimated purity by LCMS analysis was 97.6%. Analysis condition A: Retention time=1.79 min; ESI-MS(+) m/z [M+2H]²⁺: 1113.3.

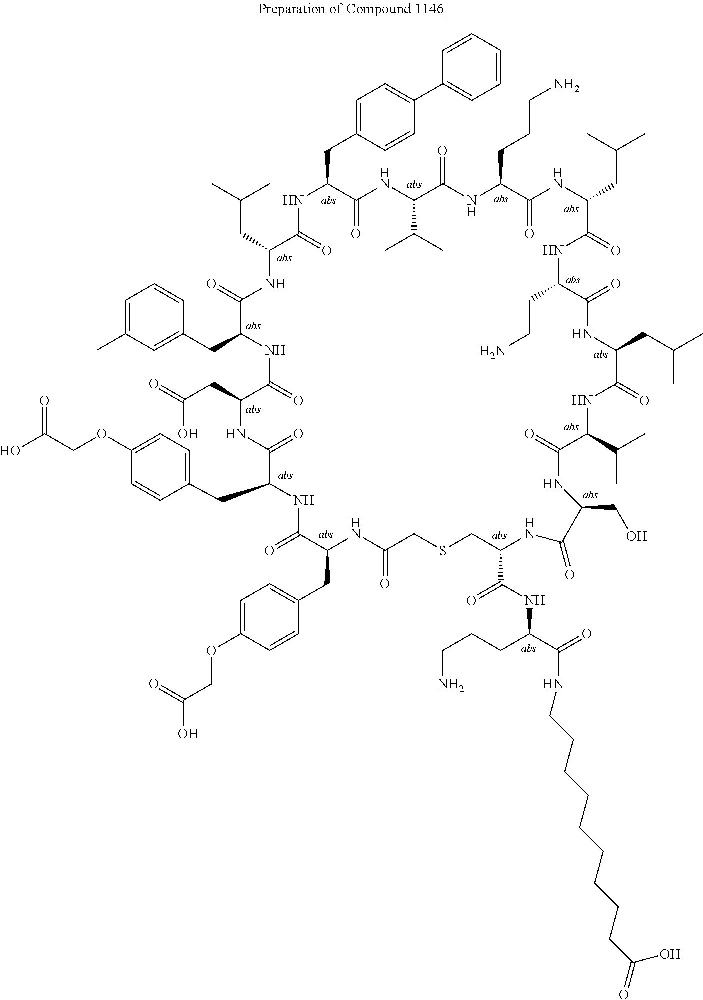 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@H](CCCN)C(=O)NCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@@H](CC(C)C)NC2=O)c1]

Compound 1146 was prepared on a 50 μmol scale. The yield of the product was 2 mg, and its estimated purity by LCMS analysis was 95.3%. Analysis condition A: Retention time=1.72 min; ESI-MS(+) m/z [M+2H]²⁺: 1120.2.

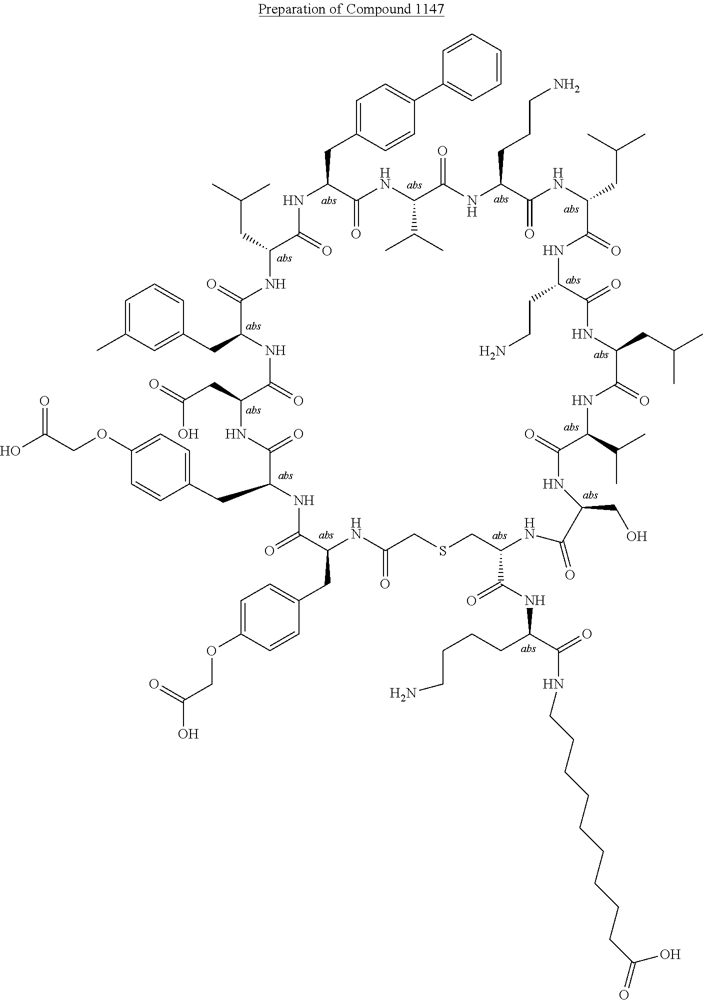 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@H](CCCCN)C(=O)NCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@@H](CC(C)C)NC2=O)c1]

Compound 1147 was prepared on a 50 μmol scale. The yield of the product was 5.1 mg, and its estimated purity by LCMS analysis was 89.8%. Analysis condition B: Retention time=1.74 min; ESI-MS(+) m/z [M+3H]³⁺: 752.1.

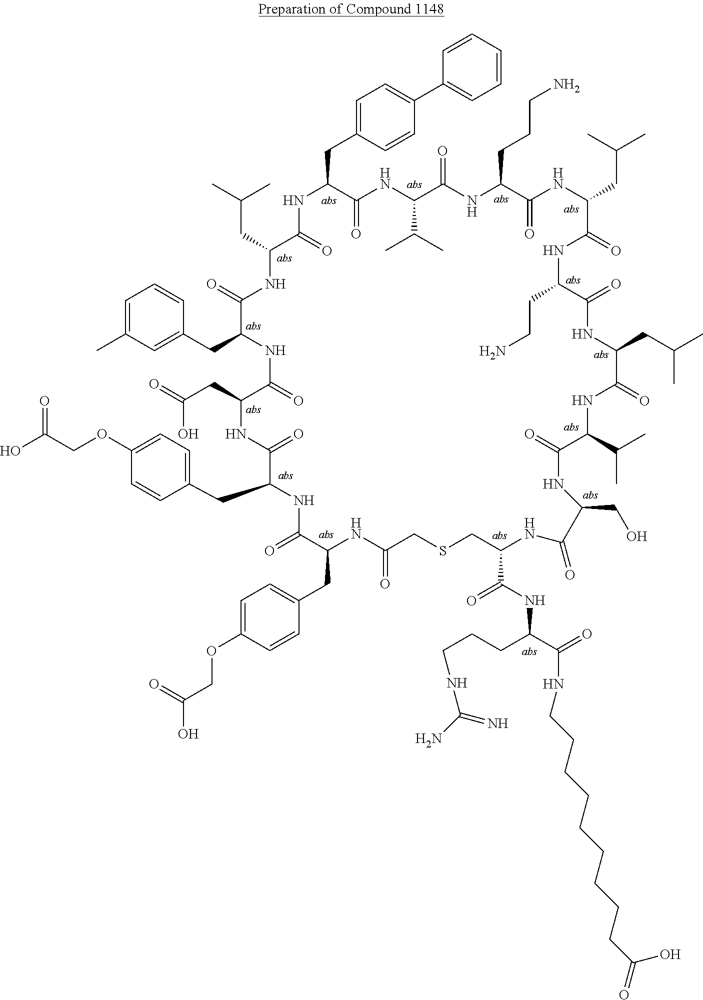 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@H](CCCNC(=N)N)C(=O)NCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@@H](CC(C)C)NC2=O)c1]

Compound 1148 was prepared on a 50 μmol scale. The yield of the product was 1.3 mg, and its estimated purity by LCMS analysis was 90.4%. Analysis condition B: Retention time=1.76 min; ESI-MS(+) m/z [M+2H]²⁺: 1141.3.

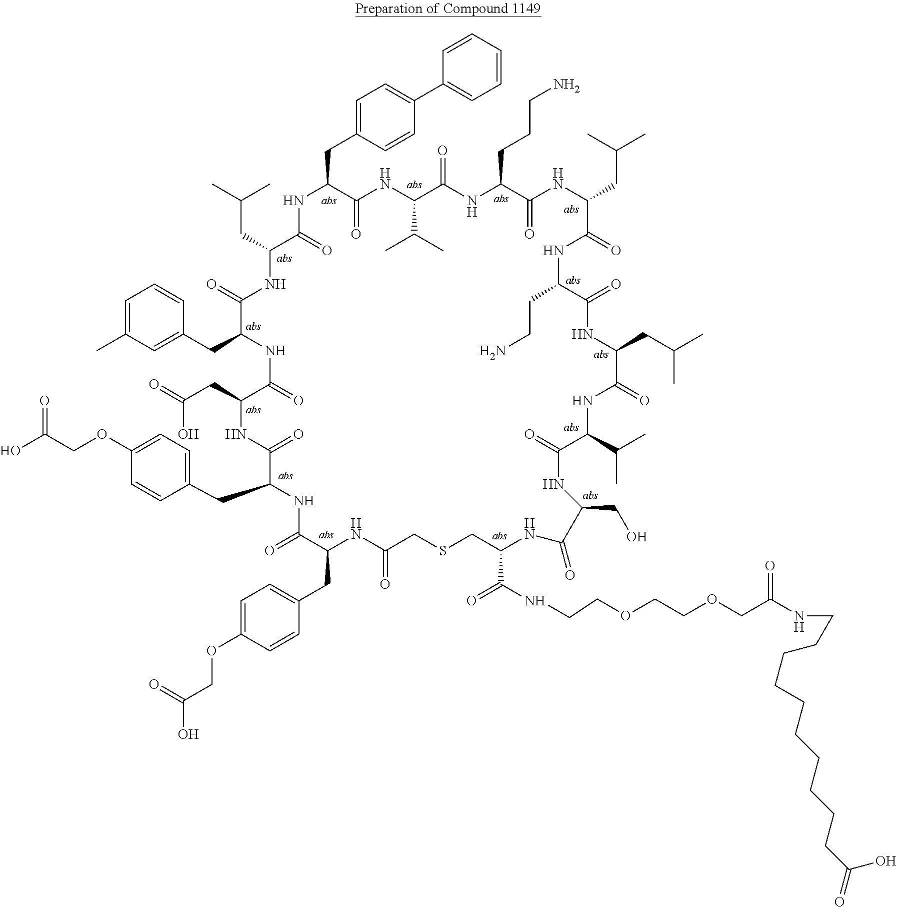 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@@H](CC(C)C)NC2=O)c1]

Compound 1149 was prepared on a 50 μmol scale. The yield of the product was 6.6 mg, and its estimated purity by LCMS analysis was 91.4%. Analysis condition A: Retention time=1.72 min; ESI-MS(+) m/z [M+2H]²⁺: 1136.1.

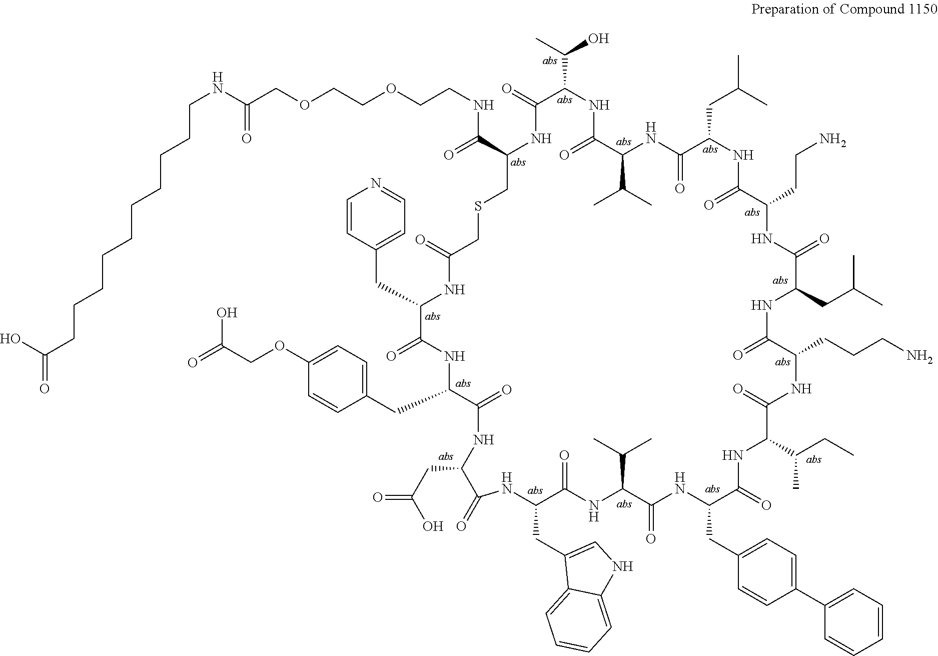 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCCOCCOCC(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1150 was prepared on a 50 μmol scale. The yield of the product was 8.7 mg, and its estimated purity by LCMS analysis was 96.2%. Analysis condition A: Retention time=1.77 min; ESI-MS(+) m/z [M+2H]²⁺: 1118.9.

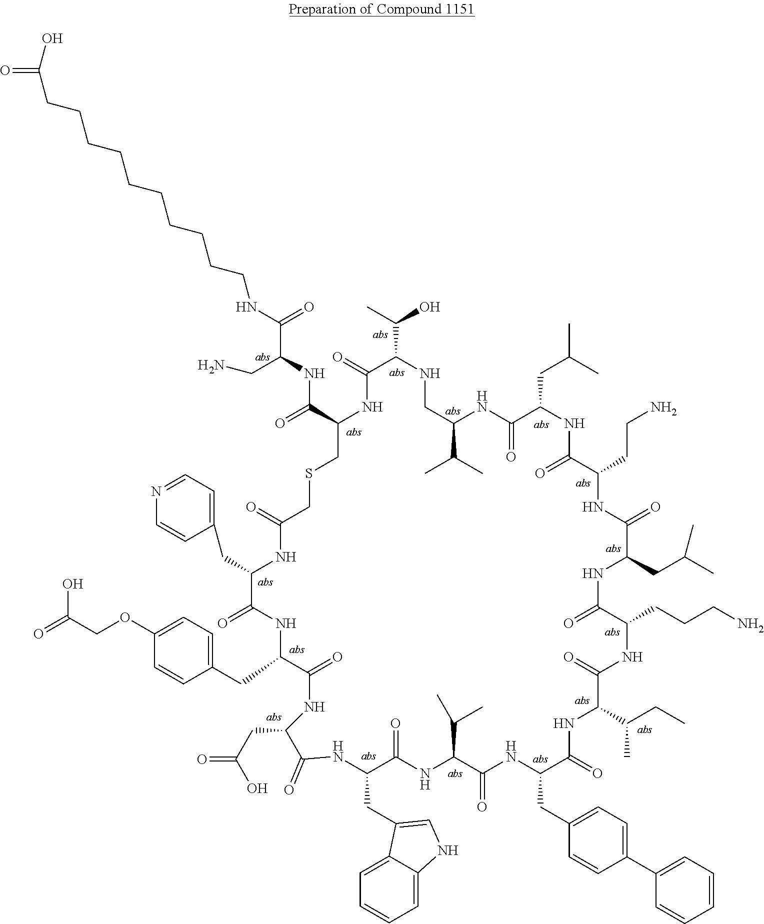 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CN)C(=O)NCCCCCCCCCCC(=O)O)NC(=O)[C@H]([C@@H](C)O)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1151 was prepared on a 50 μmol scale. The yield of the product was l mg, and its estimated purity by LCMS analysis was 96.5%. Analysis condition A: Retention time=1.58 min; ESI-MS(+) m/z [M+2H]²⁺: 1089.2.

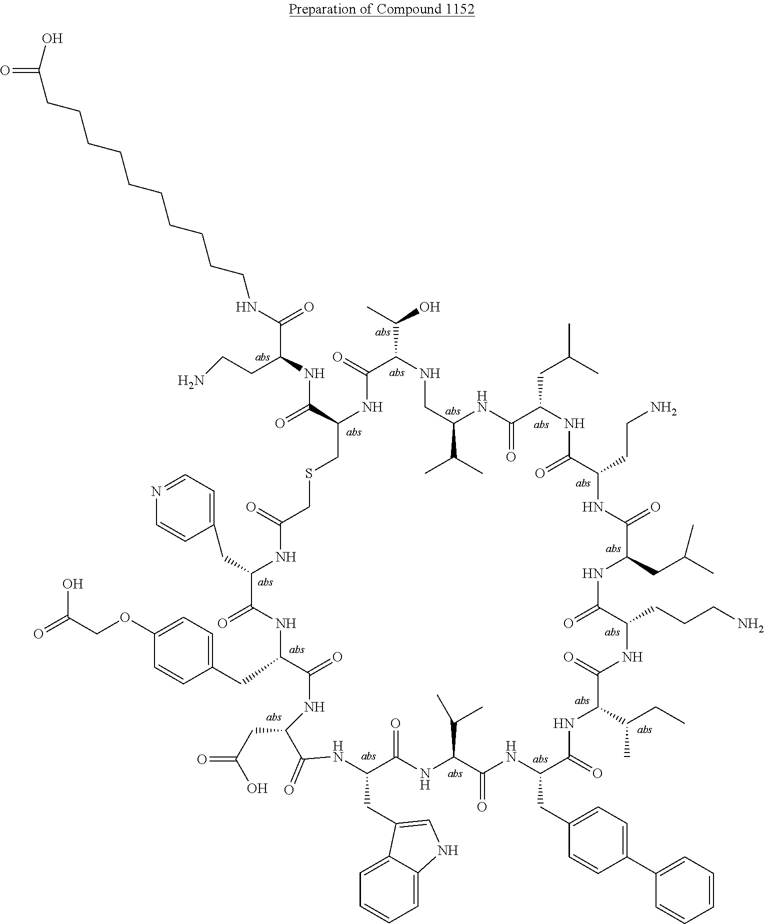 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H]([C@@H](C)O)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1152 was prepared on a 50 μmol scale. The yield of the product was 5.1 mg, and its estimated purity by LCMS analysis was 86.7%. Analysis condition A: Retention time=1.52 min; ESI-MS(+) m/z [M+3H]³⁺: 731.1.

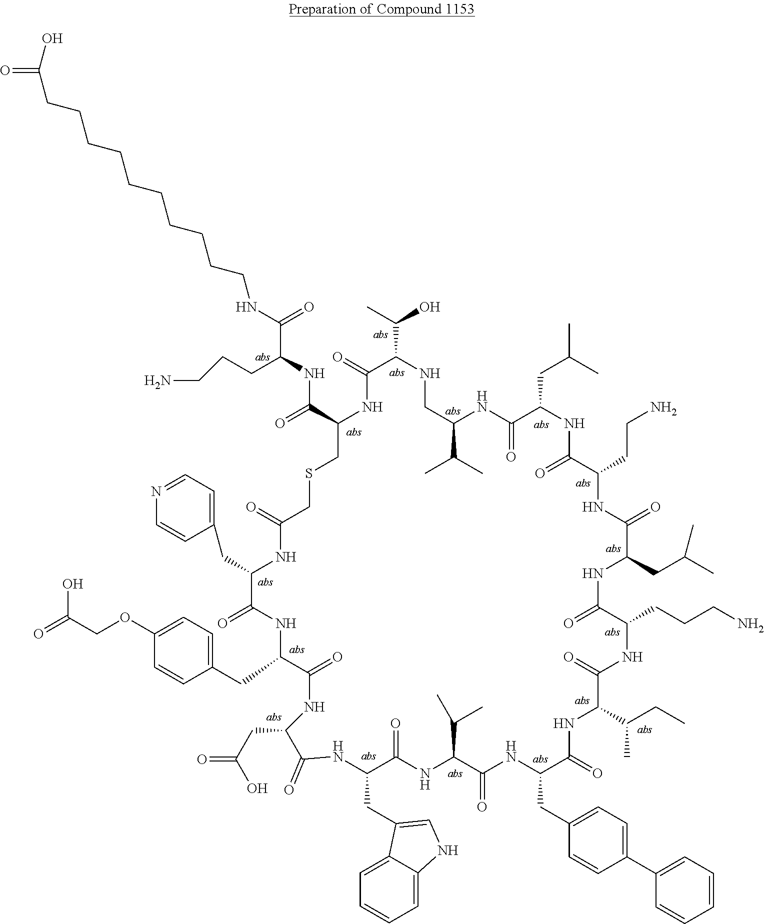 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCCCCCCCCCC(=O)O)NC(=O)[C@H]([C@@H](C)O)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1153 was prepared on a 50 μmol scale. The yield of the product was 3.9 mg, and its estimated purity by LCMS analysis was 96.1%. Analysis condition B: Retention time=1.45 min; ESI-MS(+) m/z [M+2H]²⁺: 1103.2.

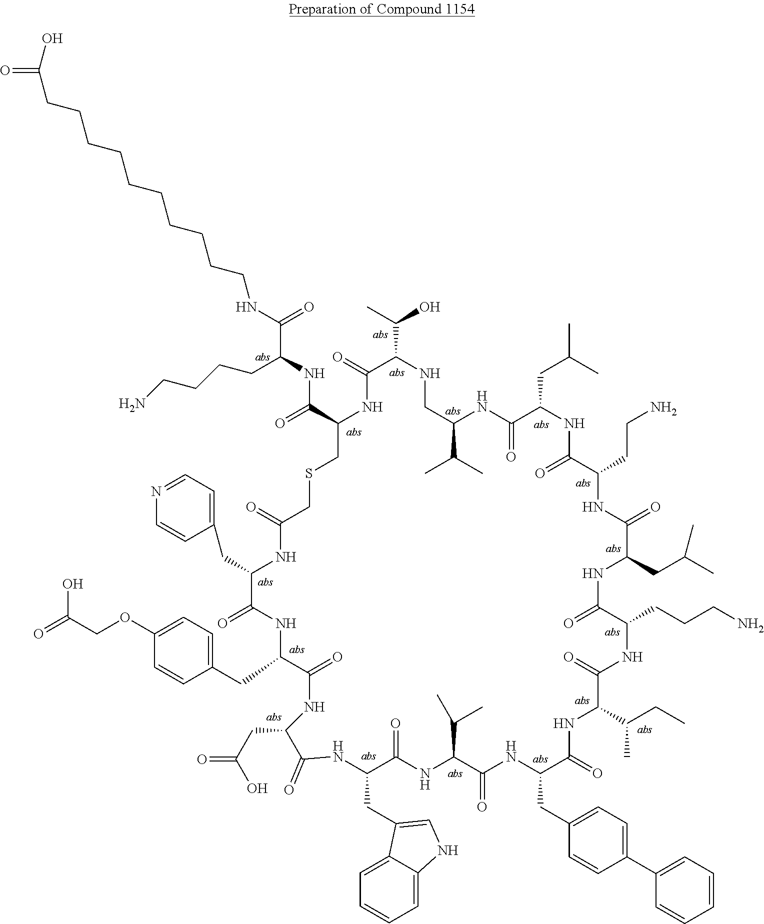 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCCN)C(=O)NCCCCCCCCCCC(=O)O)NC(=O)[C@H]([C@@H](C)O)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1154 was prepared on a 50 μmol scale. The yield of the product was 4.5 mg, and its estimated purity by LCMS analysis was 80.2%. Analysis condition B: Retention time=1.47 min; ESI-MS(+) m/z [M+2H]²⁺: 1110.9.

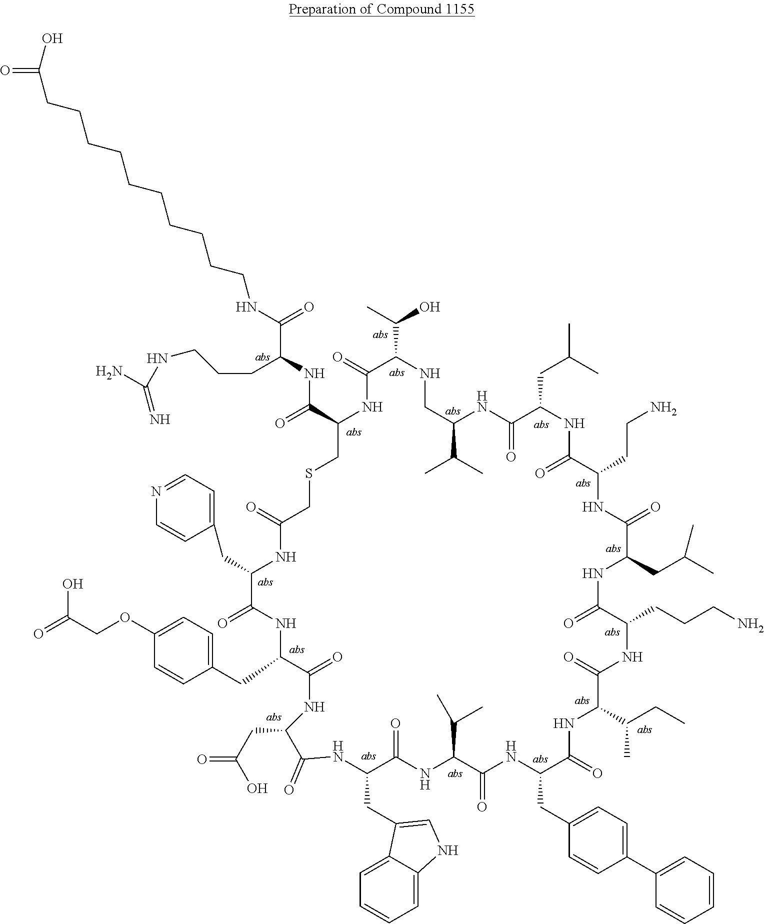 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCNC(=N)N)C(=O)NCCCCCCCCCCC(=O)O)NC(=O)[C@H]([C@@H](C)O)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1155 was prepared on a 50 μmol scale. The yield of the product was 0.7 mg, and its estimated purity by LCMS analysis was 96%. Analysis condition A: Retention time=1.5 min; ESI-MS(+) m/z [M+3H]³⁺: 750.3.

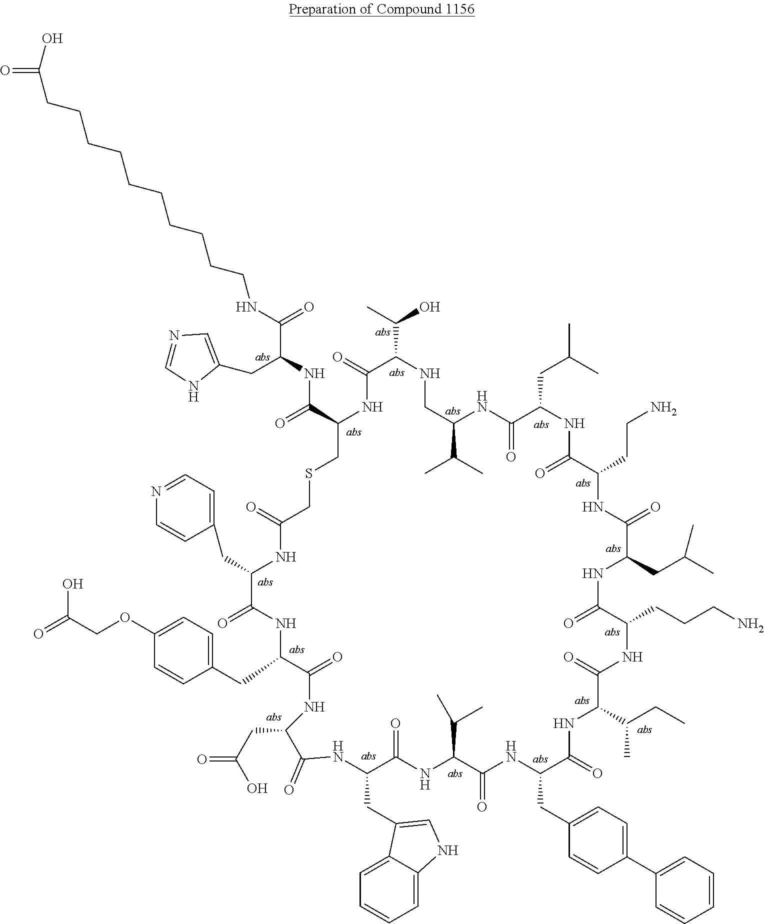 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](Cc2cnc[nH]2)C(=O)NCCCCCCCCCCC(=O)O)NC(=O)[C@H]([C@@H](C)O)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1156 was prepared on a 50 μmol scale. The yield of the product was 1.4 mg, and its estimated purity by LCMS analysis was 94.8%. Analysis condition A: Retention time=1.65 min; ESI-MS(+) m/z [M+2H]²⁺: 1115.1.

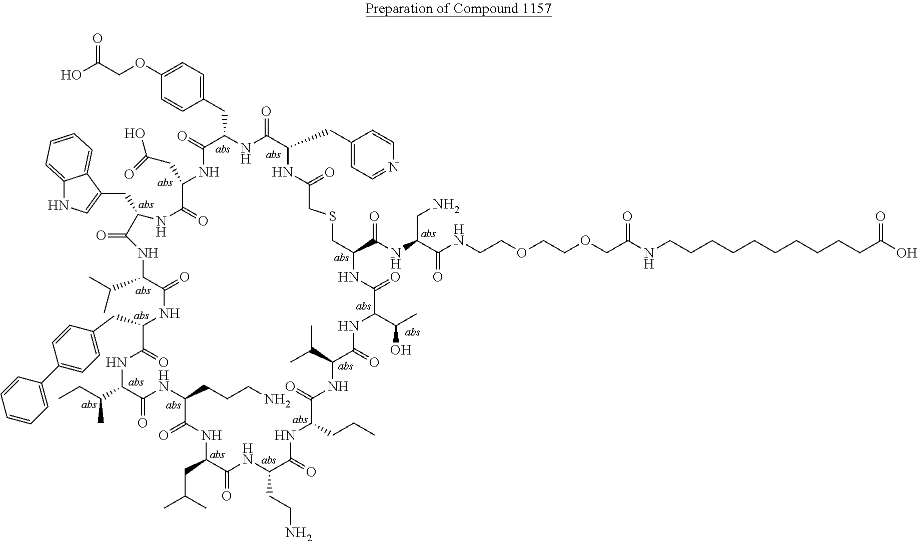 [SMILES: CCC[C@@H]1NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CN)C(=O)NCCOCCOCC(=O)NCCCCCCCCCCC(=O)O)NC(=O)C([C@@H](C)O)NC(=O)[C@H](C(C)C)NC1=O]

Compound 1157 was prepared on a 50 μmol scale. The yield of the product was 8.6 mg, and its estimated purity by LCMS analysis was 98.3%. Analysis condition B: Retention time=1.55 min; ESI-MS(+) m/z [M+3H]³⁺: 775.1.

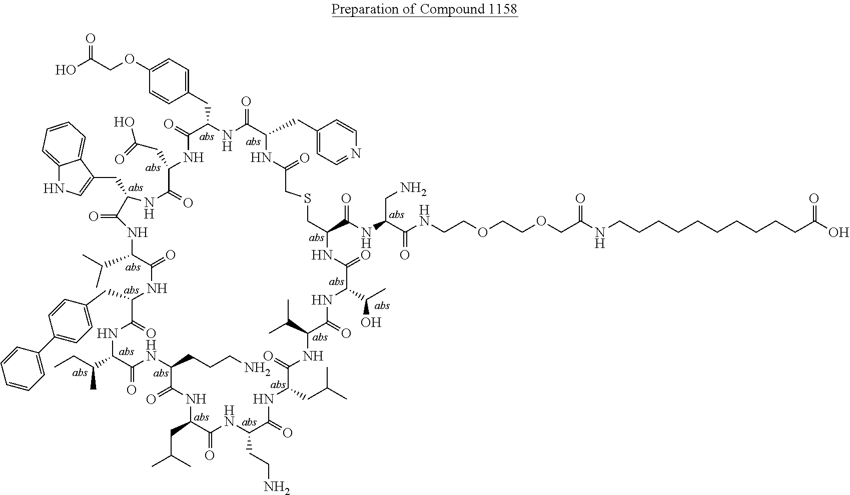 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CN)C(=O)NCCOCCOCC(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1158 was prepared on a 50 μmol scale. The yield of the product was 3.8 mg, and its estimated purity by LCMS analysis was 92.1%. Analysis condition B: Retention time=1.61 min; ESI-MS(+) m/z [M+2H]²⁺: 1169.

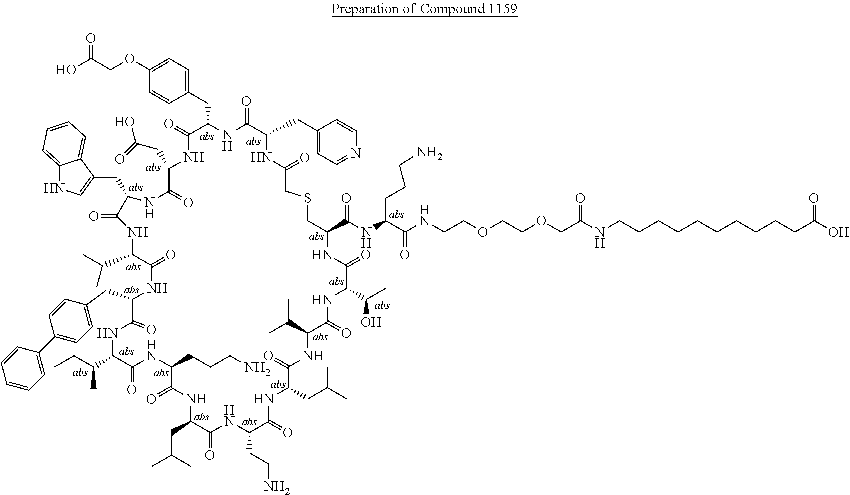 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1159 was prepared on a 50 μmol scale. The yield of the product was 7.4 mg, and its estimated purity by LCMS analysis was 86.4%. Analysis condition A: Retention time=1.7 min; ESI-MS(+) m/z [M+2H]²⁺: 1175.9.

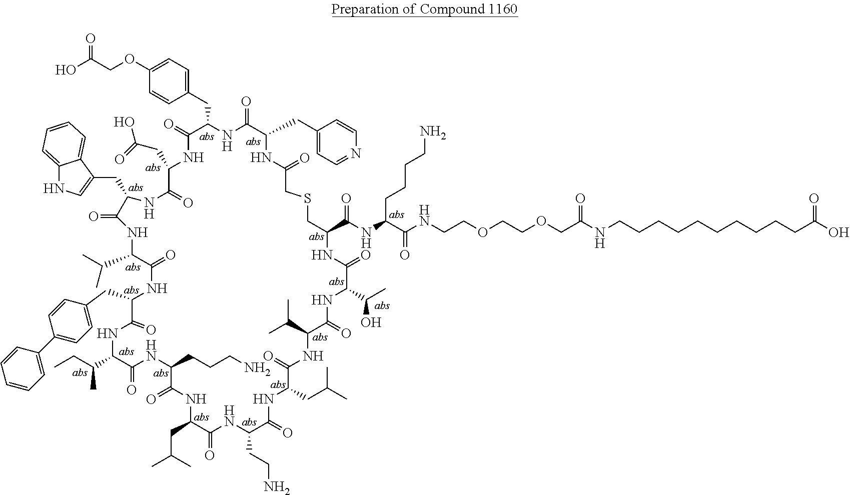 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCCN)C(=O)NCCOCCOCC(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1160 was prepared on a 50 μmol scale. The yield of the product was 7.4 mg, and its estimated purity by LCMS analysis was 91.1%. Analysis condition A: Retention time=1.71 min; ESI-MS(+) m/z [M+2H]²⁺: 1182.8.

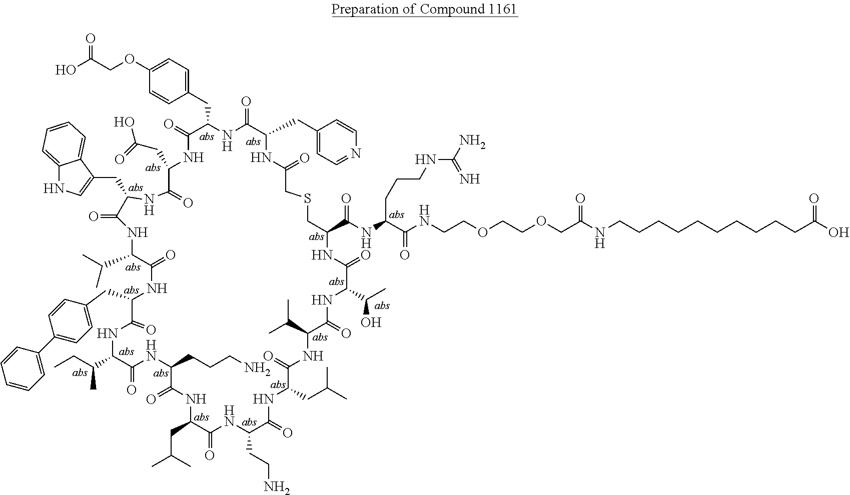 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCNC(=N)N)C(=O)NCCOCCOCC(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1161 was prepared on a 50 μmol scale. The yield of the product was 12.2 mg, and its estimated purity by LCMS analysis was 85.8%. Analysis condition B: Retention time=1.51 min; ESI-MS(+) m/z [M+2H]²⁺: 1197.2.

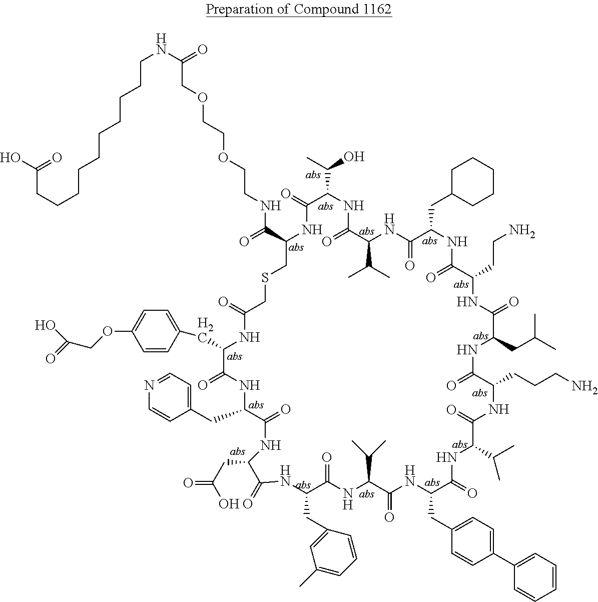 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1162 was prepared on a 50 μmol scale. The yield of the product was 2.3 mg, and its estimated purity by LCMS analysis was 87.2%. Analysis condition B: Retention time=1.64 min; ESI-MS(+) m/z [M+3H]³⁺: 746.8.

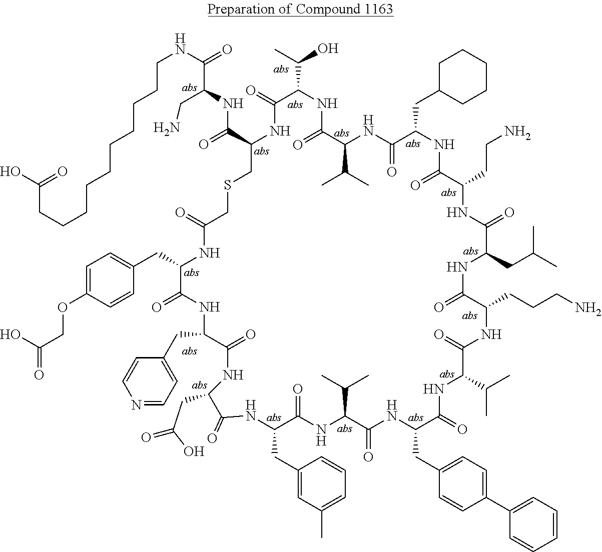 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1163 was prepared on a 50 μmol scale. The yield of the product was 2.2 mg, and its estimated purity by LCMS analysis was 85.6%. Analysis condition B: Retention time=1.58 min; ESI-MS(+) m/z [M+2H]²⁺: 1090.2.

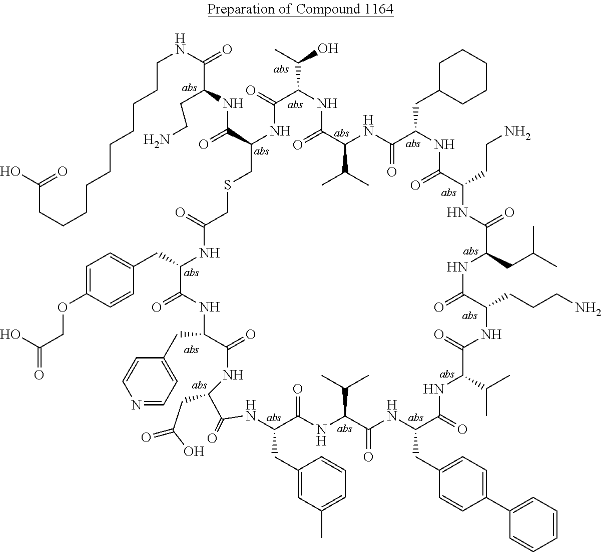 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1164 was prepared on a 50 μmol scale. The yield of the product was 2.4 mg, and its estimated purity by LCMS analysis was 91%. Analysis condition B: Retention time=1.84 min; ESI-MS(+) m/z [M+3H]³⁺: 731.6.

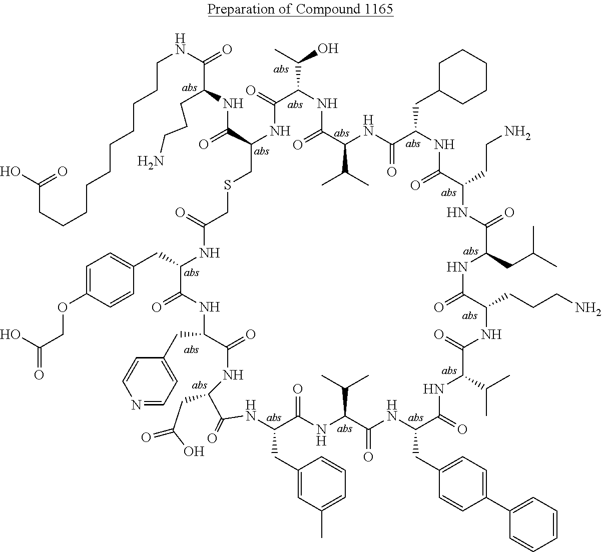 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1165 was prepared on a 50 μmol scale. The yield of the product was 0.7 mg, and its estimated purity by LCMS analysis was 93%. Analysis condition B: Retention time=1.58 min; ESI-MS(+) m/z [M+2H]²⁺: 1104.

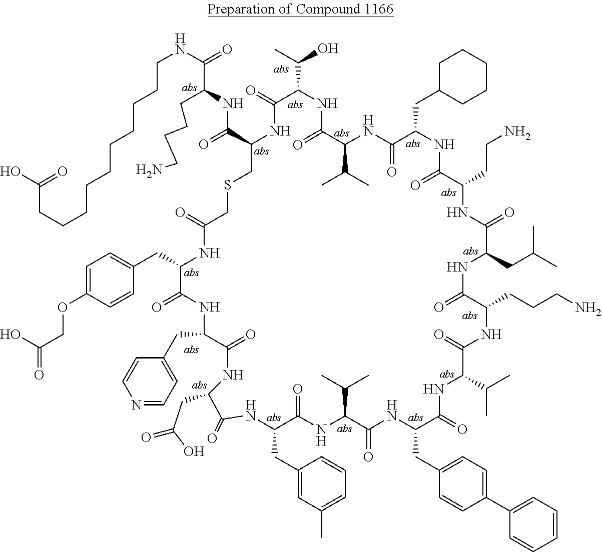 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1166 was prepared on a 50 μmol scale. The yield of the product was l mg, and its estimated purity by LCMS analysis was 100%. Analysis condition B: Retention time=1.84 min; ESI-MS(+) m/z [M+2H]²⁺: 1111.1.

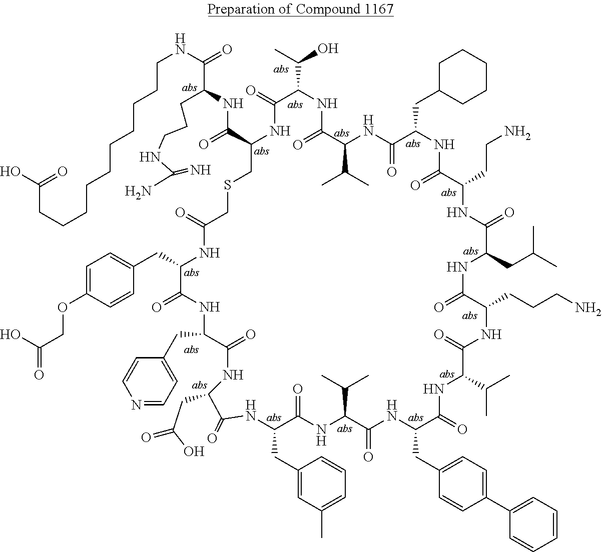 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCCNC(=N)N)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1167 was prepared on a 50 μmol scale. The yield of the product was 4.2 mg, and its estimated purity by LCMS analysis was 90.1%. Analysis condition B: Retention time=1.85 min; ESI-MS(+) m/z [M+2H]²⁺: 1125.3.

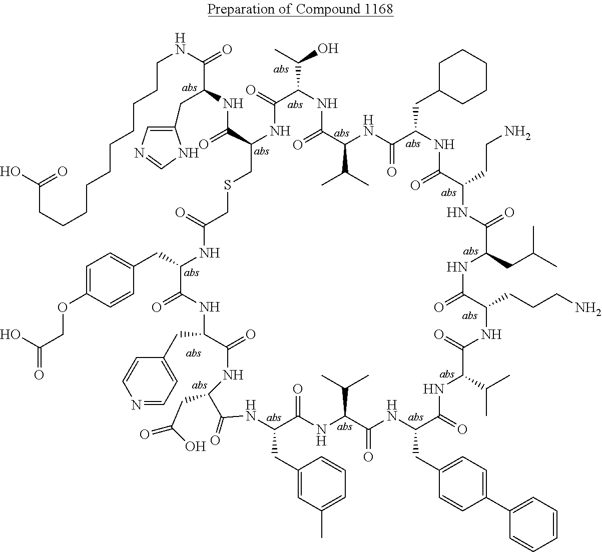 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](Cc3cnc[nH]3)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1168 was prepared on a 50 μmol scale. The yield of the product was 1 mg, and its estimated purity by LCMS analysis was 88.1%. Analysis condition B: Retention time=1.52 min; ESI-MS(+) m/z [M+3H]³⁺: 744.

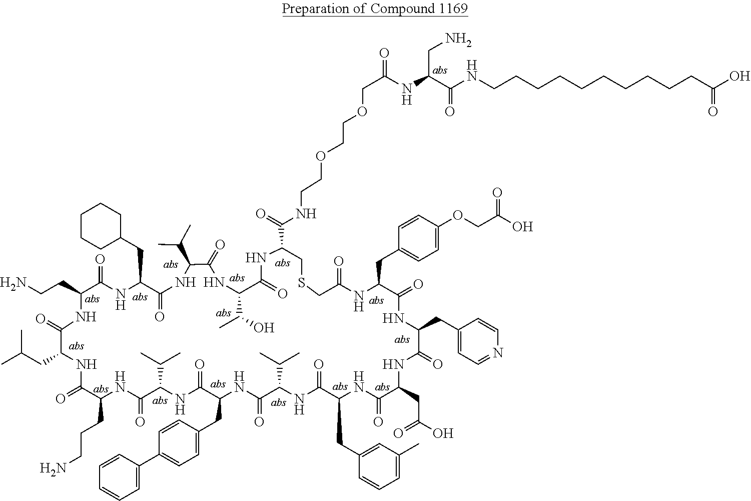 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)NCCOCCOCC(=O)N[C@@H](CN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1169 was prepared on a 50 μmol scale. The yield of the product was 3 mg, and its estimated purity by LCMS analysis was 100%. Analysis condition A: Retention time=2.04 min; ESI-MS(+) m/z [M+2H]²⁺: 1163.

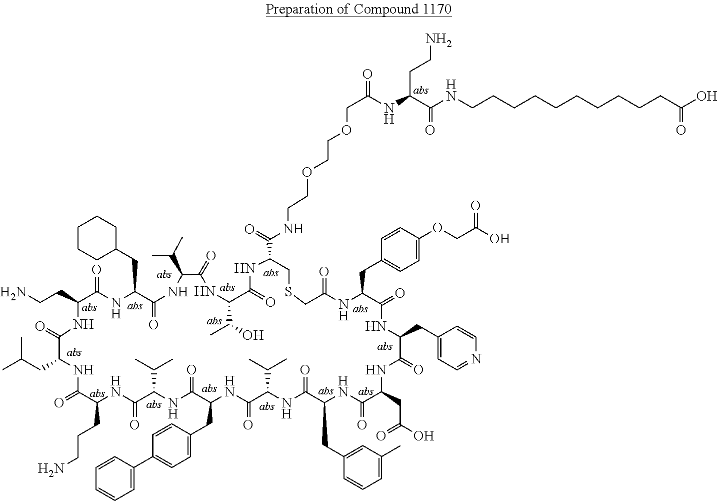 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)NCCOCCOCC(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1170 was prepared on a 50 μmol scale. The yield of the product was 8.2 mg, and its estimated purity by LCMS analysis was 97.3%. Analysis condition B: Retention time=1.5 min; ESI-MS(+) m/z [M+3H]³⁺: 780.3.

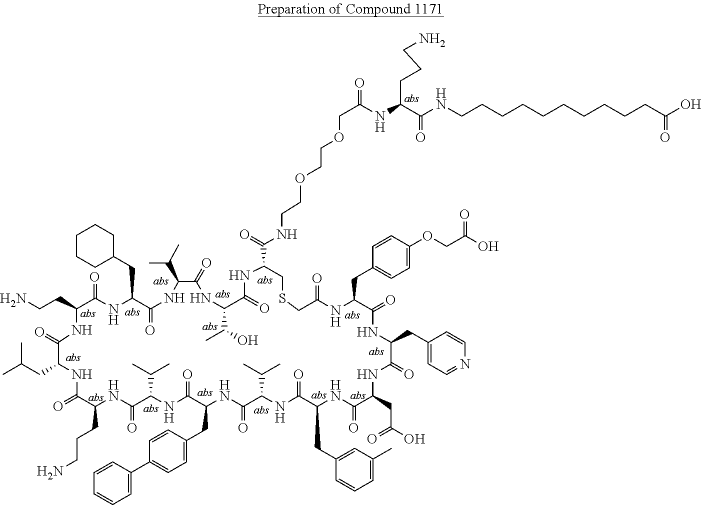 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)NCCOCCOCC(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1171 was prepared on a 50 μmol scale. The yield of the product was 9.2 mg, and its estimated purity by LCMS analysis was 85.3%. Analysis condition A: Retention time=2.03 min; ESI-MS(+) m/z [M+2H]²⁺: 1176.3.

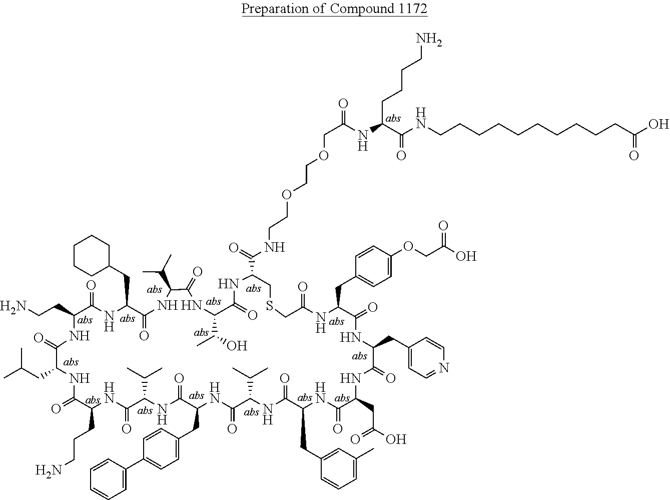 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)NCCOCCOCC(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1172 was prepared on a 50 μmol scale. The yield of the product was 4.8 mg, and its estimated purity by LCMS analysis was 100%. Analysis condition A: Retention time=1.97 min; ESI-MS(+) m/z [M+2H]²⁺: 1183.1.

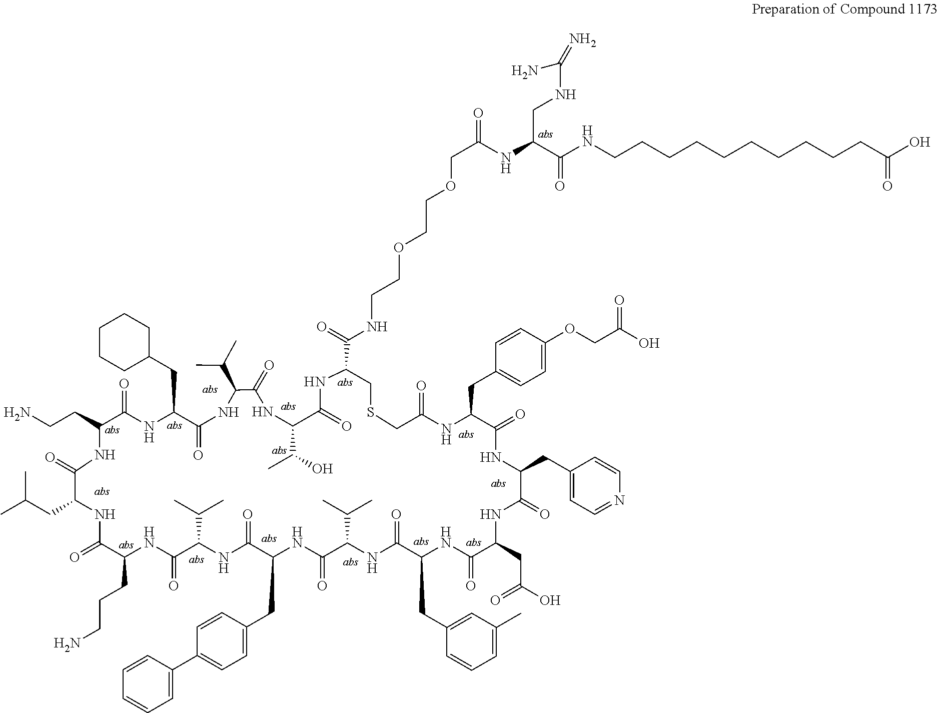

Compound 1173 was prepared on a 50 μmol scale. The yield of the product was 4.9 mg, and its estimated purity by LCMS analysis was 89.5%. Analysis condition B: Retention time=1.53 min; ESI-MS(+) m/z [M+3H]³⁺: 798.5.

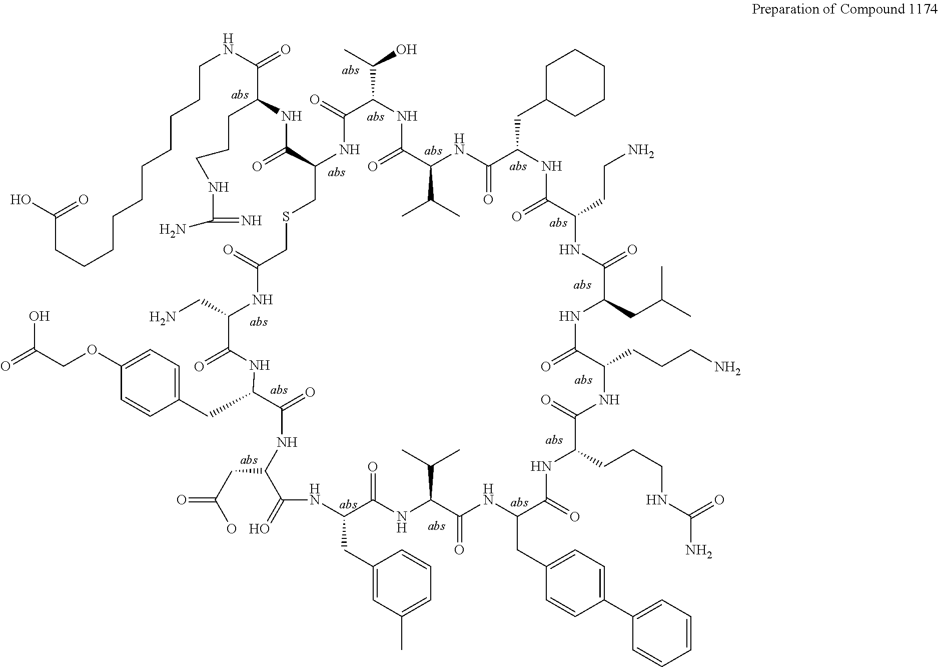

Compound 1174 was prepared on a 50 μmol scale. The yield of the product was 10.4 mg, and its estimated purity by LCMS analysis was 95.7%. Analysis condition B: Retention time=1.48 min; ESI-MS(+) m/z [M+3H]³⁺: 749.

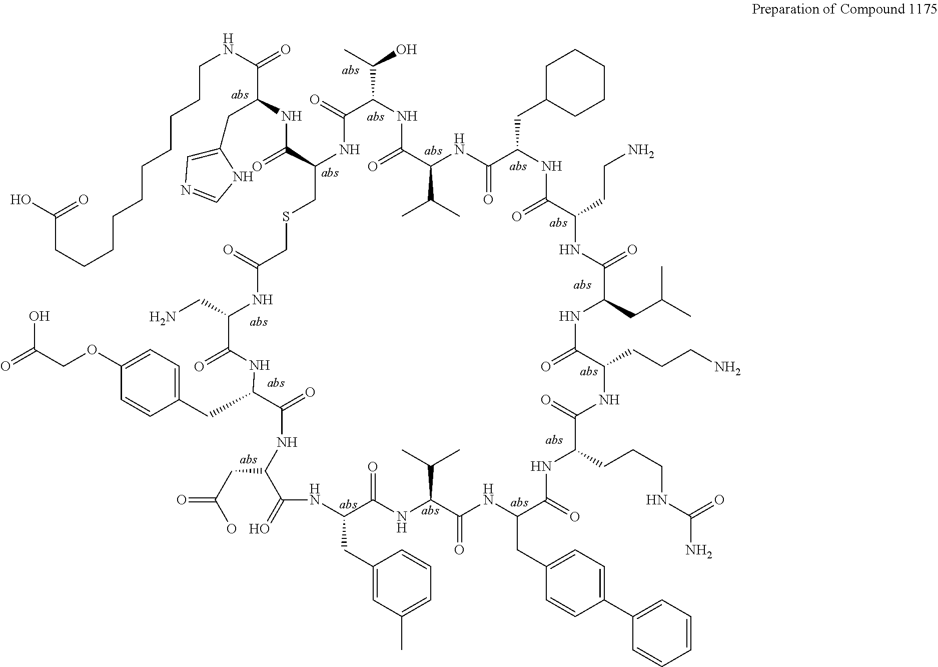

Compound 1175 was prepared on a 50 μmol scale. The yield of the product was 4.2 mg, and its estimated purity by LCMS analysis was 96.4%. Analysis condition A: Retention time=1.69 min; ESI-MS(+) m/z [M+3H]³⁺: 742.9.

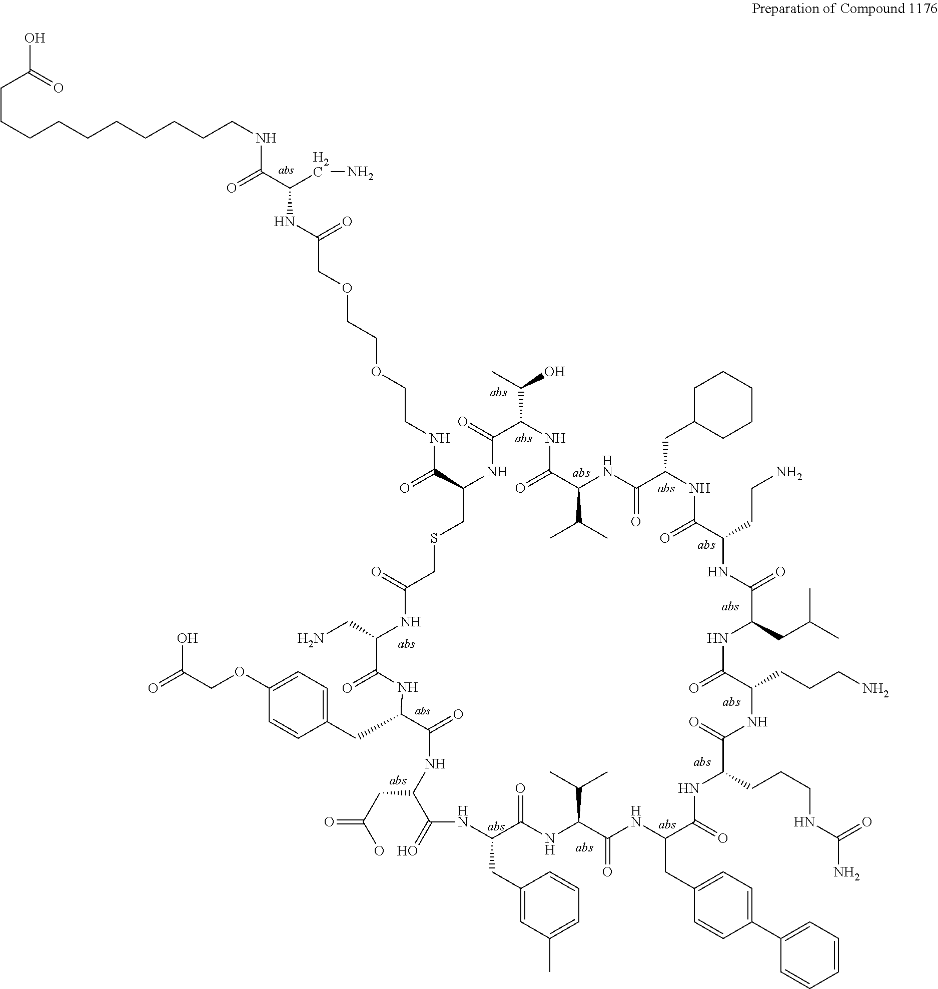

Compound 1176 was prepared on a 50 μmol scale. The yield of the product was 14.1 mg, and its estimated purity by LCMS analysis was 85.7%. Analysis condition B: Retention time=1.58 min; ESI-MS(+) m/z [M+2H]²⁺: 1160.9.

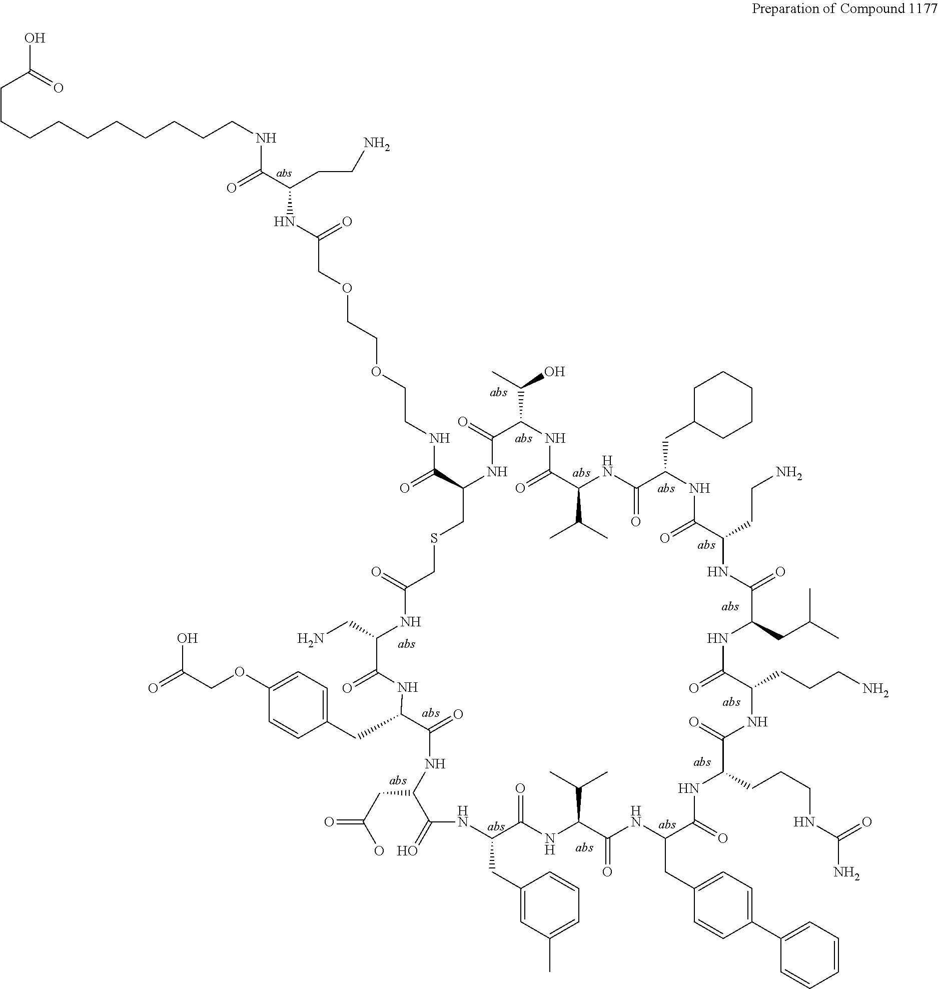

Compound 1177 was prepared on a 50 μmol scale. The yield of the product was 12.1 mg, and its estimated purity by LCMS analysis was 84.6%. Analysis condition B: Retention time=1.51 min; ESI-MS(+) m/z [M+3H]³⁺: 779.

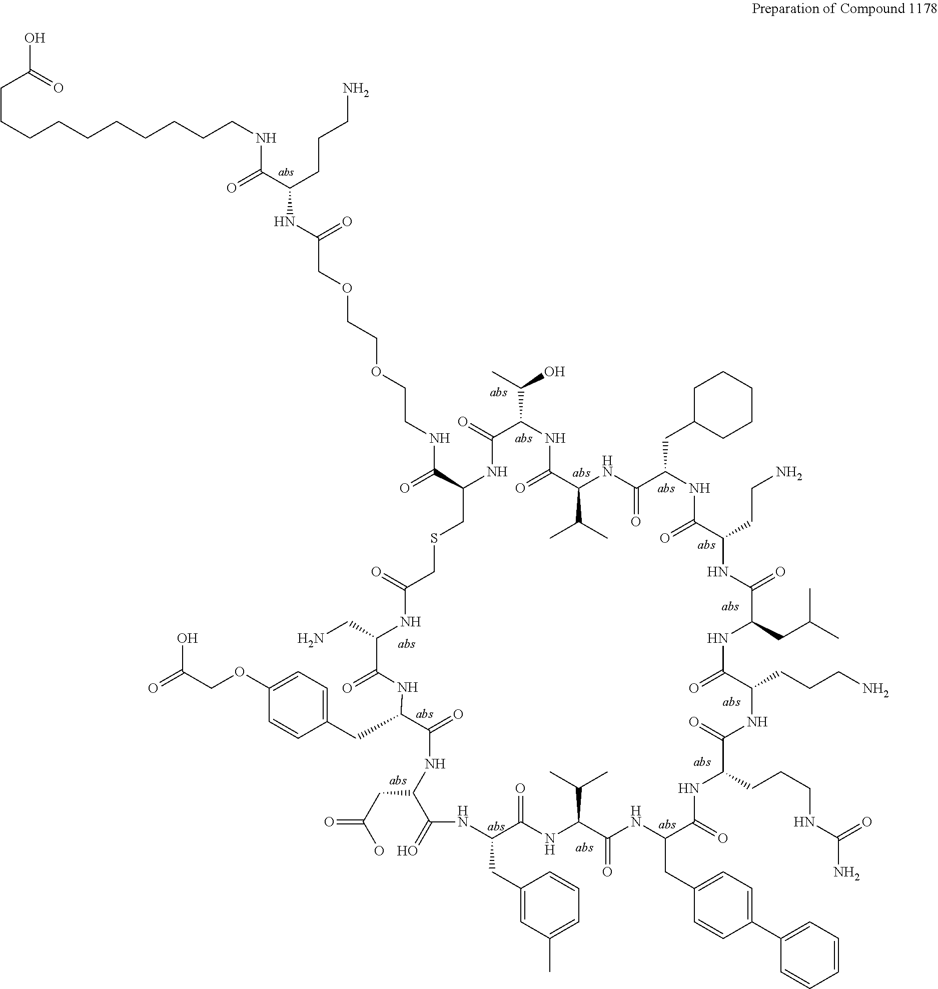

Compound 1178 was prepared on a 50 μmol scale. The yield of the product was 12.4 mg, and its estimated purity by LCMS analysis was 98.3%. Analysis condition B: Retention time=1.56 min; ESI-MS(+) m/z [M+3H]³⁺: 783.2.

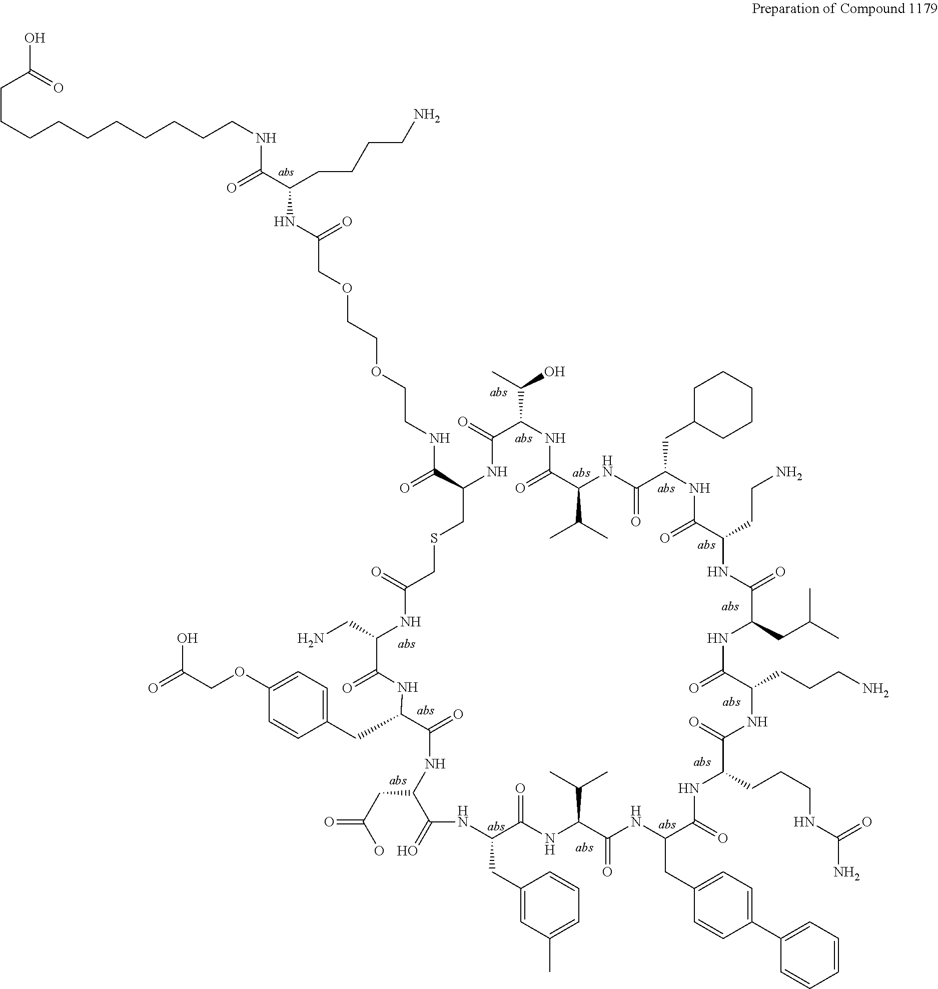

Compound 1179 was prepared on a 50 μmol scale. The yield of the product was 14.7 mg, and its estimated purity by LCMS analysis was 100%. Analysis condition A: Retention time=1.48 min; ESI-MS(+) m/z [M+3H]³⁺: 788.2.

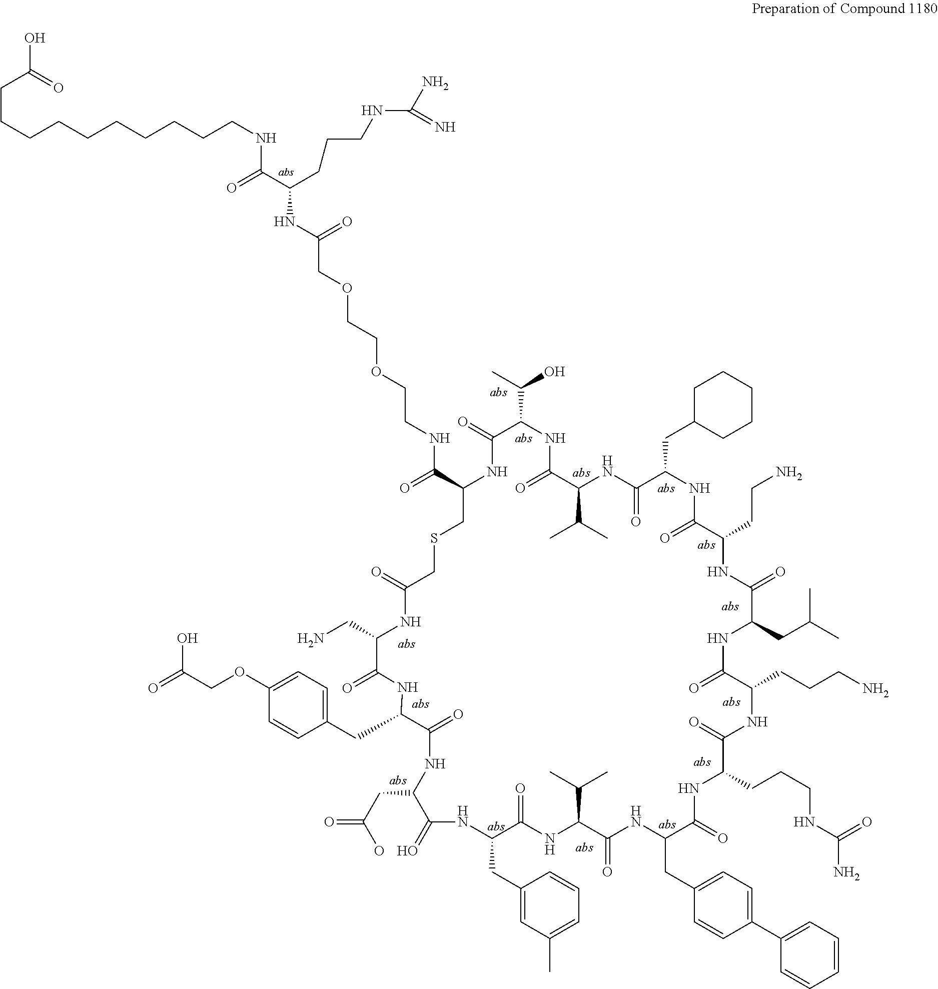

Compound 1180 was prepared on a 50 μmol scale. The yield of the product was 21.2 mg, and its estimated purity by LCMS analysis was 98%. Analysis condition A: Retention time=1.49 min; ESI-MS(+) m/z [M+3H]³⁺: 798.2.

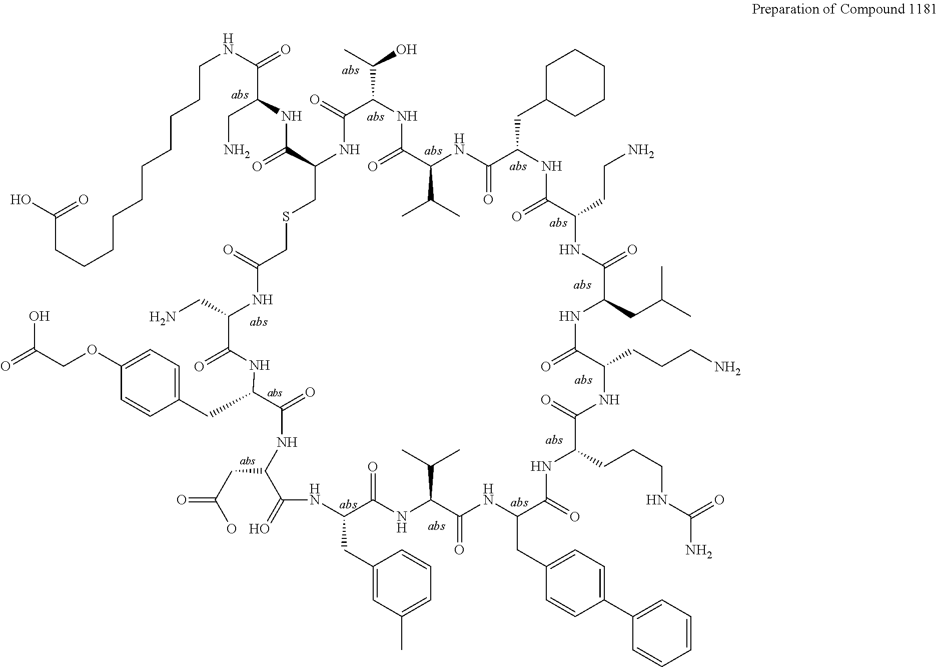

Compound 1181 was prepared on a 50 μmol scale. The yield of the product was 3 mg, and its estimated purity by LCMS analysis was 100%. Analysis condition A: Retention time=1.71 min; ESI-MS(+) m/z [M+2H]²⁺: 1088.

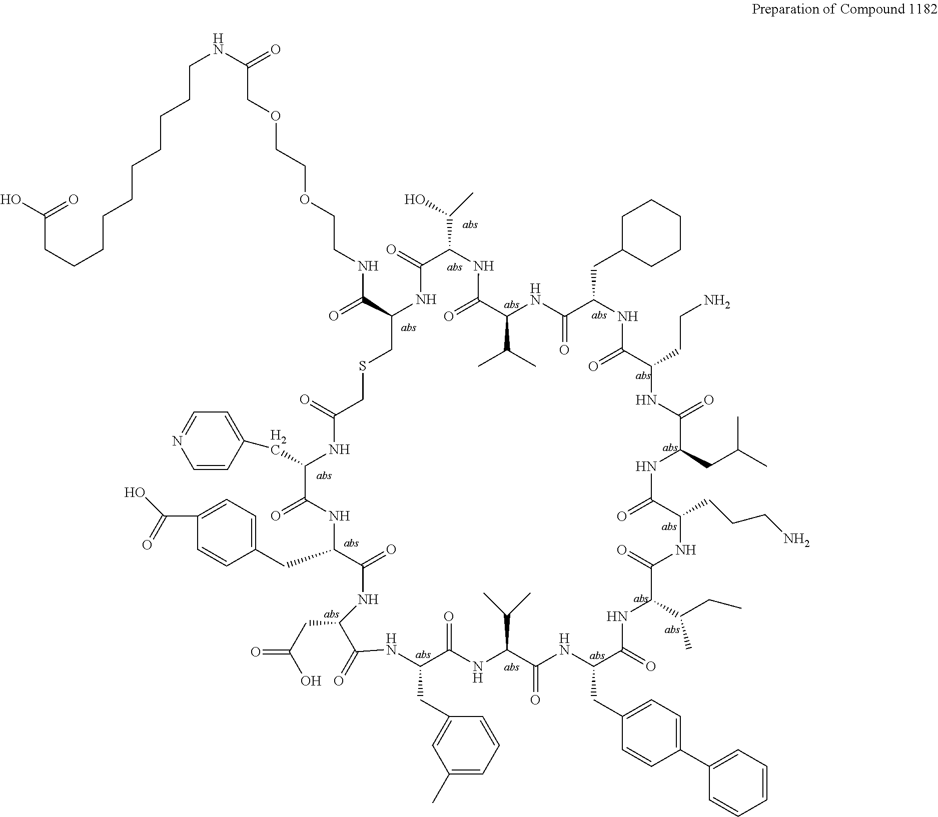 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)NCCOCCOCC(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1182 was prepared on a 50 μmol scale. The yield of the product was 1.4 mg, and its estimated purity by LCMS analysis was 94.3%. Analysis condition A: Retention time=1.85 min; ESI-MS(+) m/z [M+2H]²⁺: 1111.7.

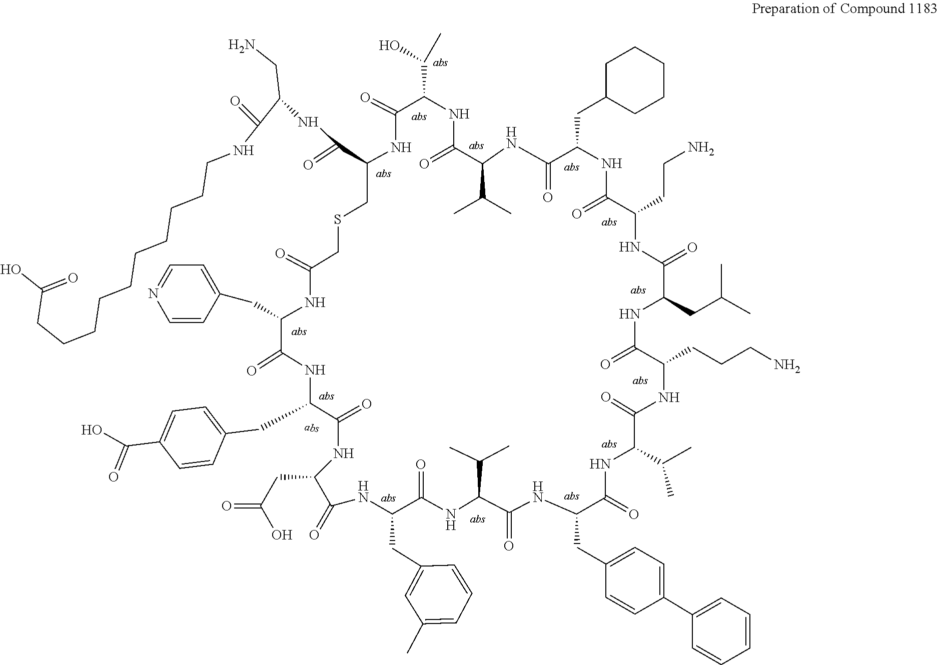

Compound 1183 was prepared on a 50 μmol scale. The yield of the product was 2.4 mg, and its estimated purity by LCMS analysis was 91.6%. Analysis condition B: Retention time=1.58 min; ESI-MS(+) m/z [M+2H]²⁺: 1082.

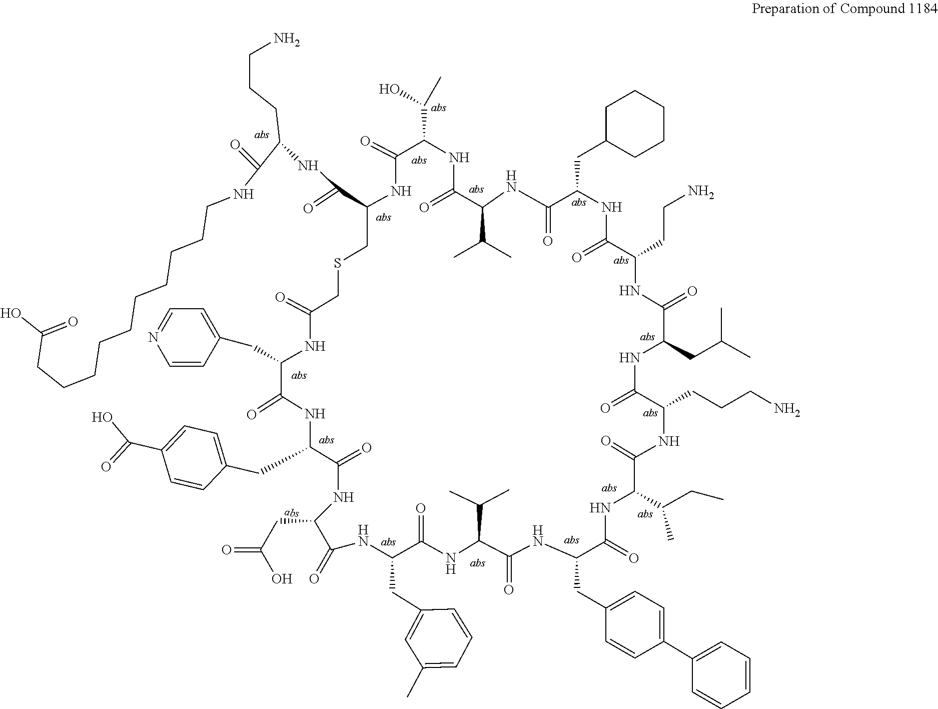 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1184 was prepared on a 50 μmol scale. The yield of the product was 3.7 mg, and its estimated purity by LCMS analysis was 85.4%. Analysis condition B: Retention time=1.55 min; ESI-MS(+) m/z [M+3H]³⁺: 731.

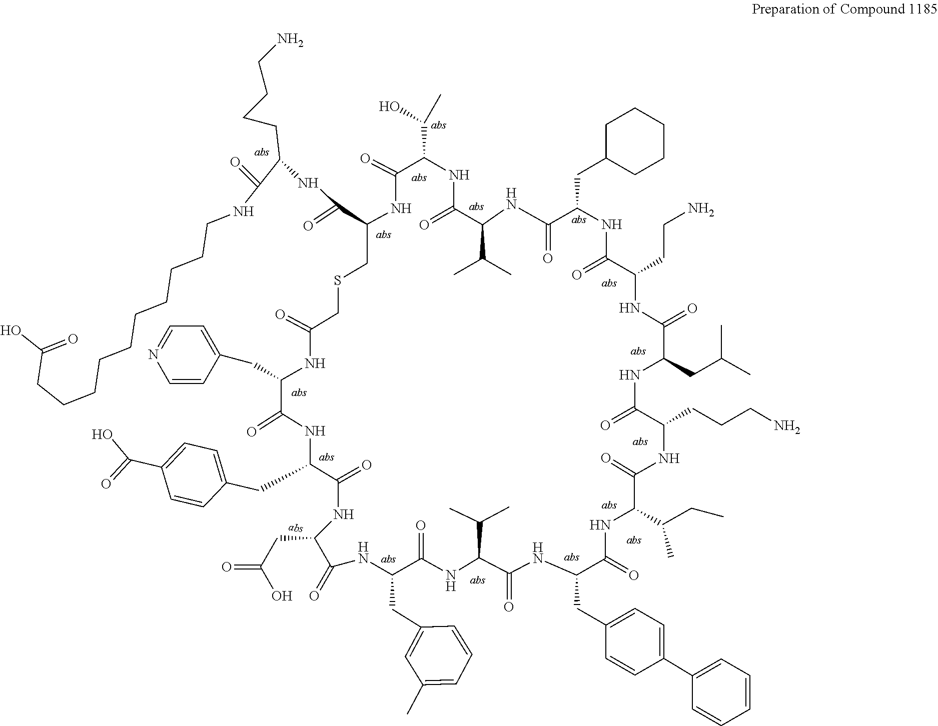 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1185 was prepared on a 50 μmol scale. The yield of the product was 5.9 mg, and its estimated purity by LCMS analysis was 87.2%. Analysis condition A: Retention time=1.8 min; ESI-MS(+) m/z [M+2H]²⁺: 1103.2.

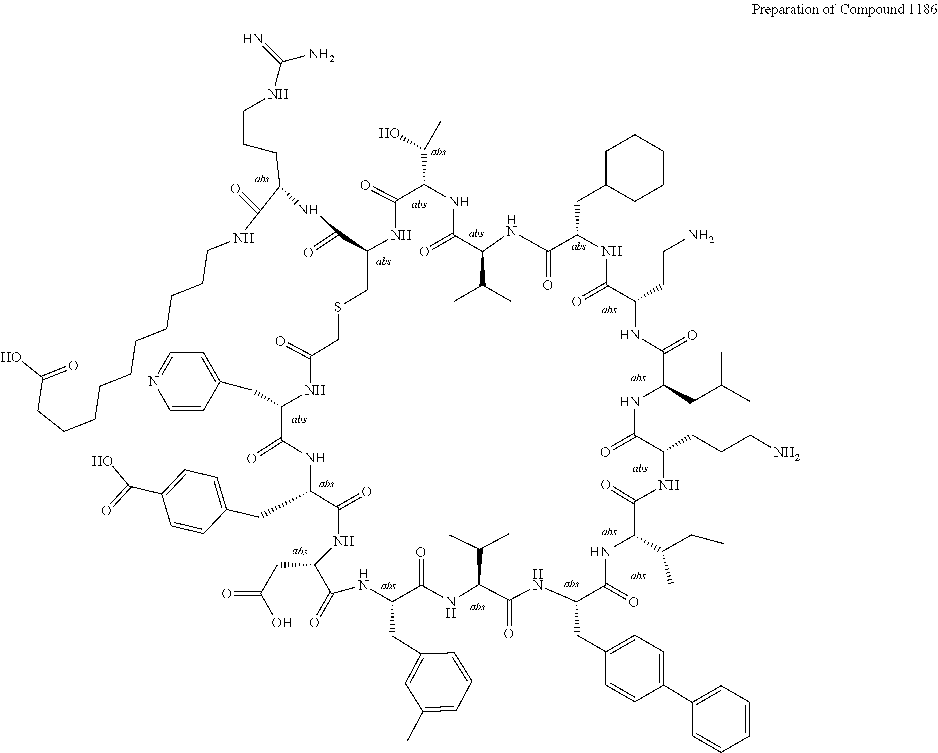 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCCNC(=N)N)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1186 was prepared on a 50 μmol scale. The yield of the product was 3.9 mg, and its estimated purity by LCMS analysis was 96.9%. Analysis condition B: Retention time=1.56 min; ESI-MS(+) m/z [M+3H]³⁺: 745.

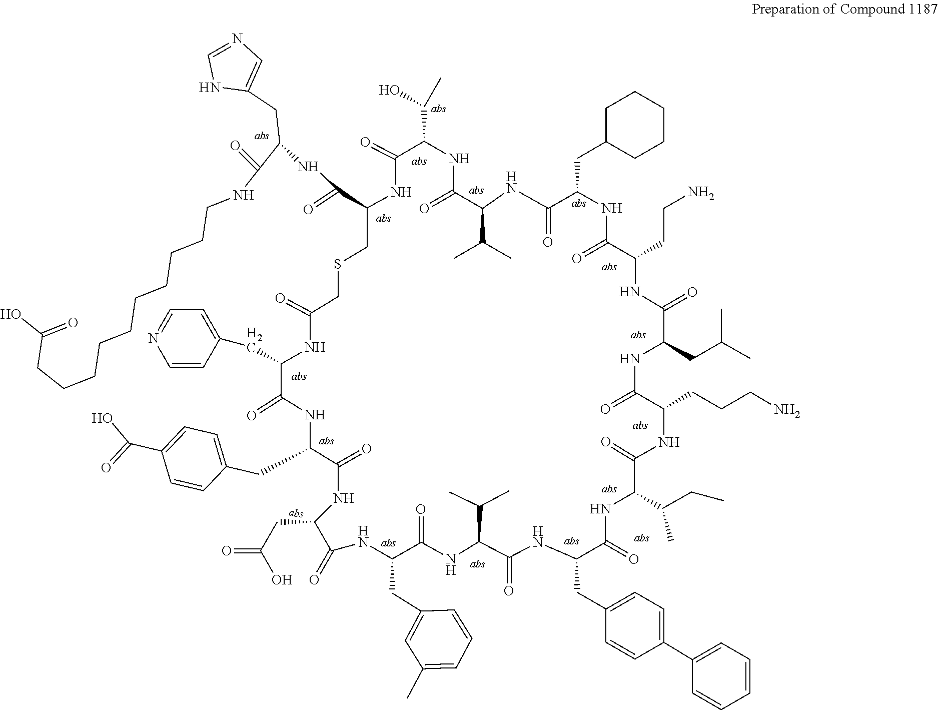 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](Cc2cnc[nH]2)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1187 was prepared on a 50 μmol scale. The yield of the product was 1.9 mg, and its estimated purity by LCMS analysis was 95.2%. Analysis condition A: Retention time=1.77 min; ESI-MS(+) m/z [M+2H]²⁺: 1107.4.

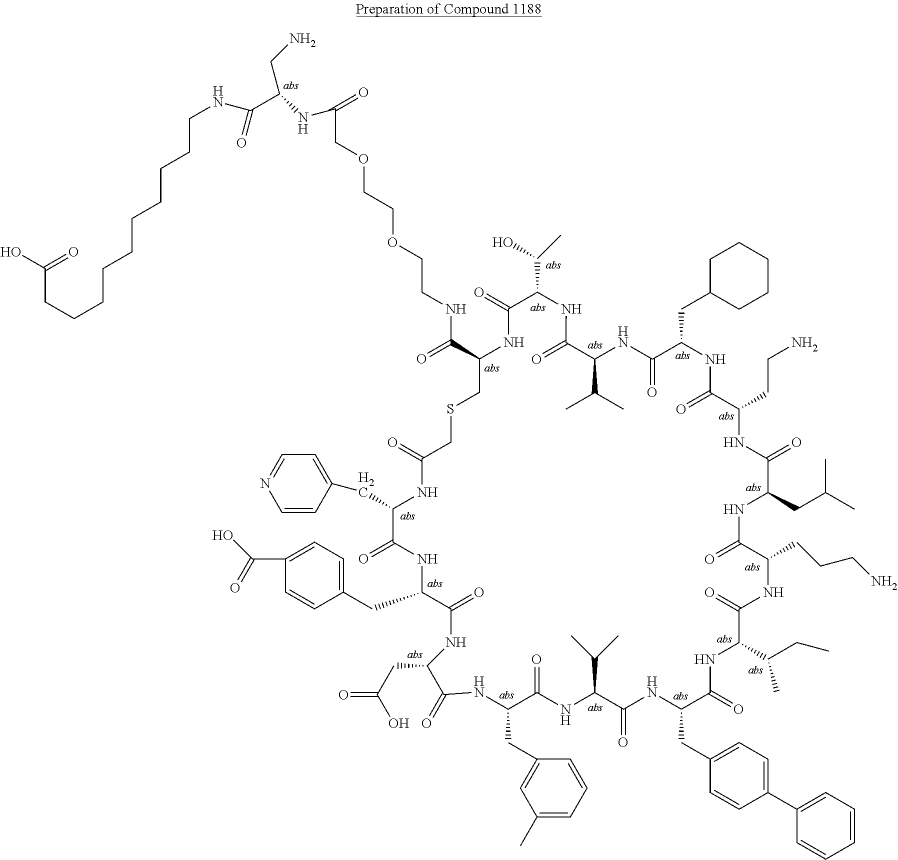 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)NCCOCCOCC(=O)N[C@@H](CN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1188 was prepared on a 50 μmol scale. The yield of the product was 4.4 mg, and its estimated purity by LCMS analysis was 98.5%. Analysis condition A: Retention time=1.76 min; ESI-MS(+) m/z [M+3H]³⁺: 770.1.

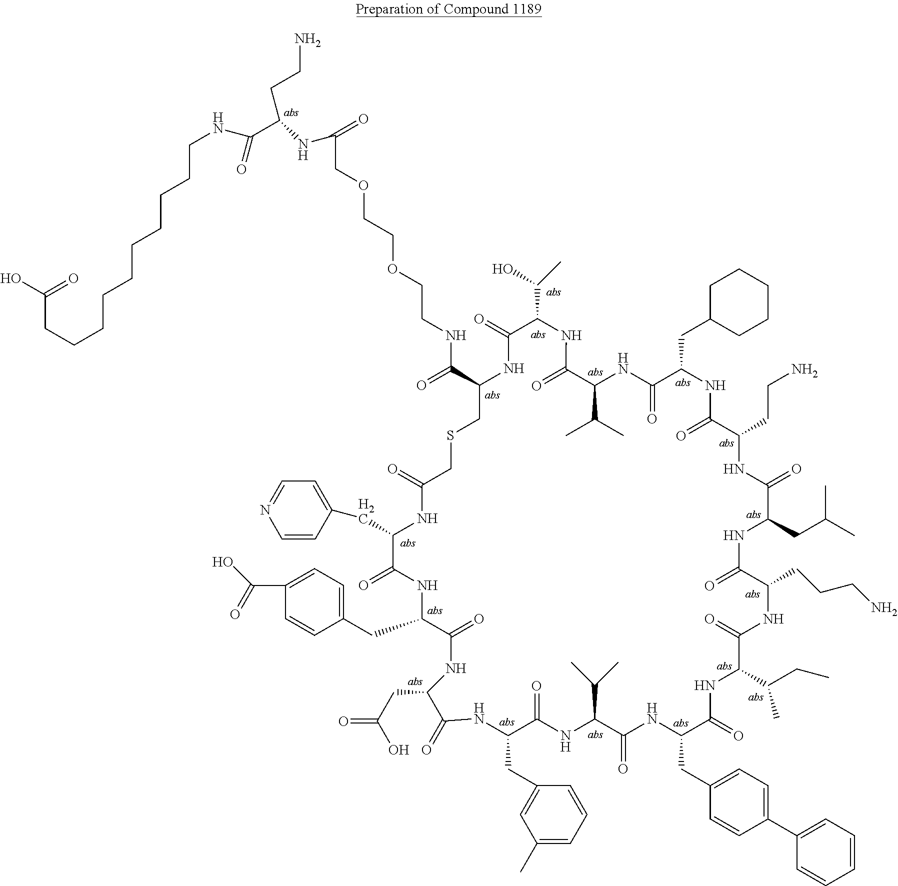 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)NCCOCCOCC(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1189 was prepared on a 50 μmol scale. The yield of the product was 12.6 mg, and its estimated purity by LCMS analysis was 85.5%. Analysis condition A: Retention time=1.75 min; ESI-MS(+) m/z [M+3H]³⁺: 775.1.

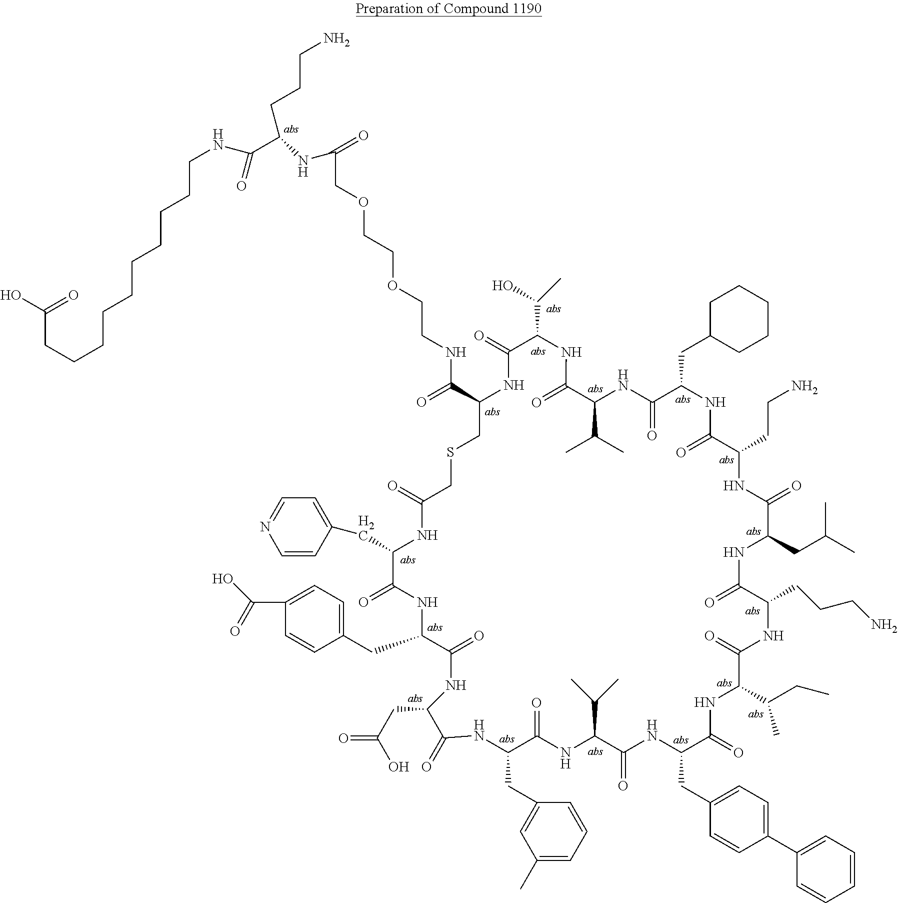 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)NCCOCCOCC(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1190 was prepared on a 50 μmol scale. The yield of the product was 8.8 mg, and its estimated purity by LCMS analysis was 85.6%. Analysis condition B: Retention time=1.62 min; ESI-MS(+) m/z [M+3H]³⁺: 779.1.

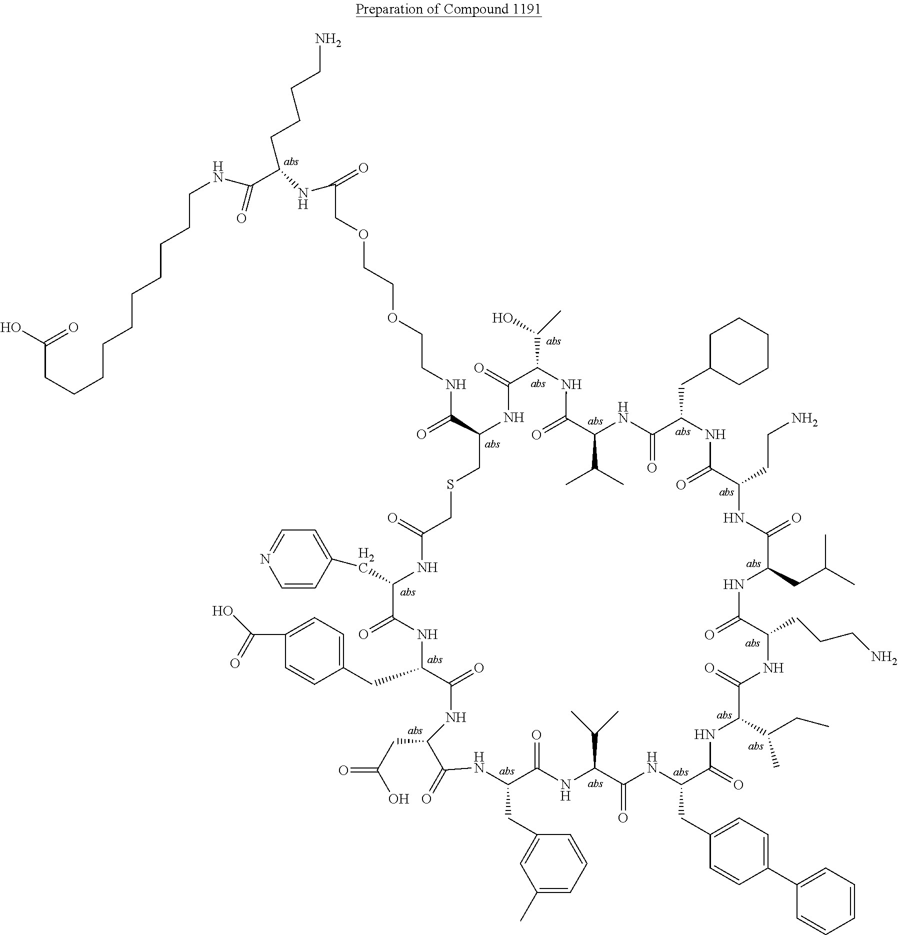 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)NCCOCCOCC(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1191 was prepared on a 50 μmol scale. The yield of the product was 13.5 mg, and its estimated purity by LCMS analysis was 85.7%. Analysis condition B: Retention time=1.6 min; ESI-MS(+) m/z [M+3H]³⁺: 784.1.

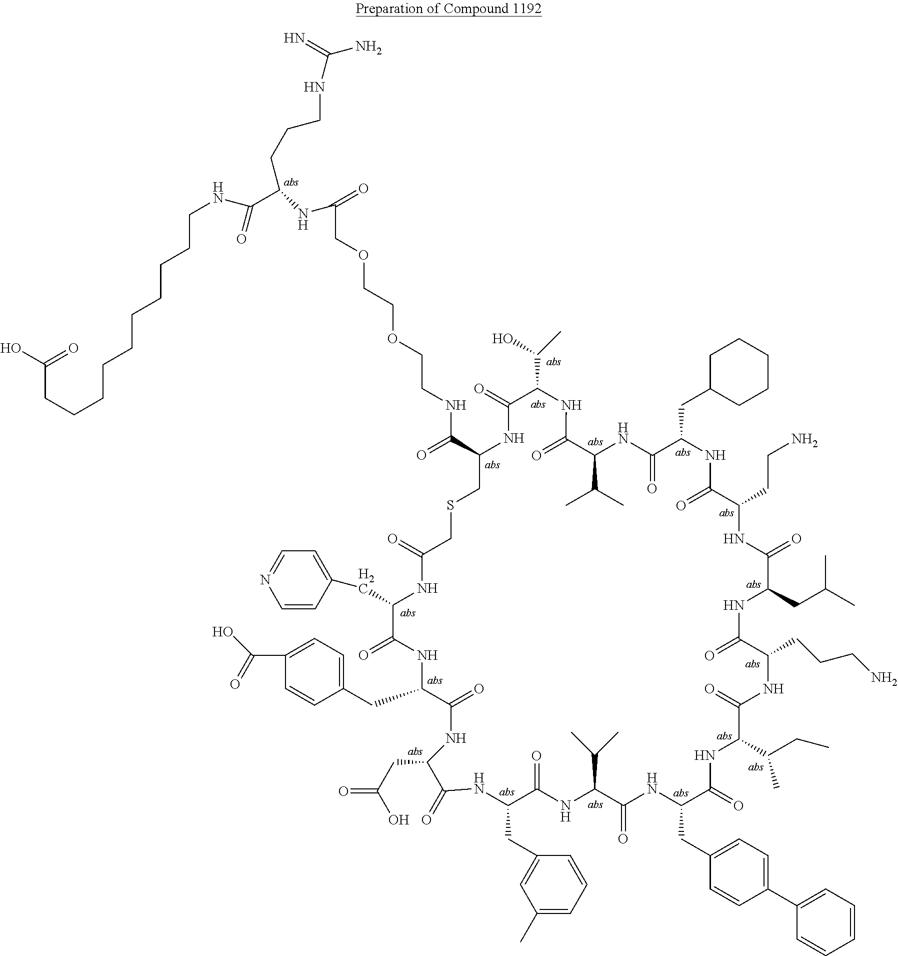 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)NCCOCCOCC(=O)N[C@@H](CCCNC(=N)N)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1192 was prepared on a 50 μmol scale. The yield of the product was 9.8 mg, and its estimated purity by LCMS analysis was 92%. Analysis condition A: Retention time=1.74 min; ESI-MS(+) m/z [M+2H]²⁺: 1190.

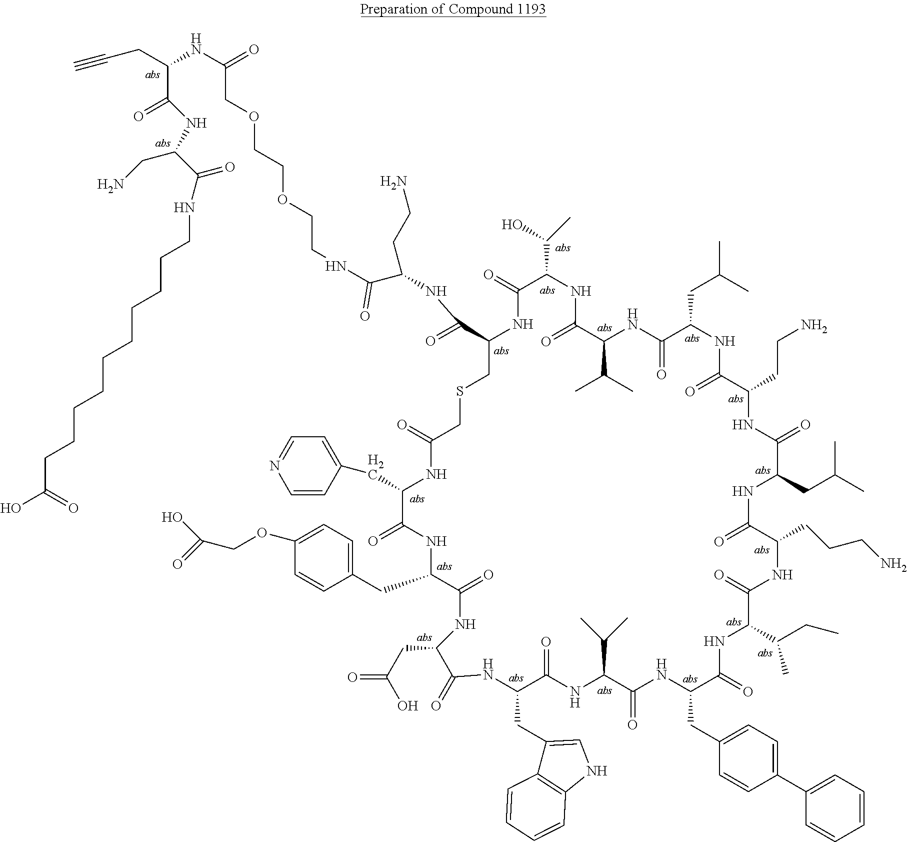 [SMILES: C#CC[C@H](NC(=O)COCCOCCNC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CN)C(=O)NCCCCCCCCCCC(=O)O]

Compound 1193 was prepared on a 50 μmol scale. The yield of the product was 9 mg, and its estimated purity by LCMS analysis was 94%. Analysis condition B: Retention time=1.48 min; ESI-MS(+) m/z [M+2H]²⁺: 1259.

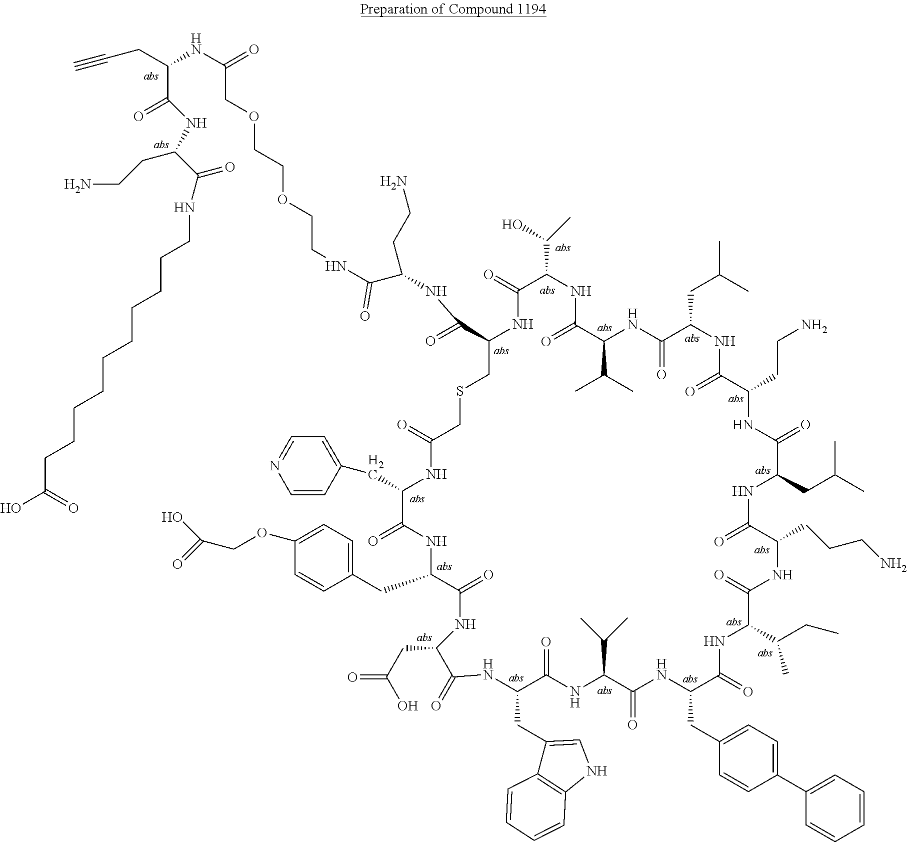 [SMILES: C#CC[C@H](NC(=O)COCCOCCNC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCN)C(=O)NCCCCCCCCCCC(=O)O]

Compound 1194 was prepared on a 50 μmol scale. The yield of the product was 5.8 mg, and its estimated purity by LCMS analysis was 95.9%. Analysis condition B: Retention time=1.49 min; ESI-MS(+) m/z [M+2H]²⁺: 1267.

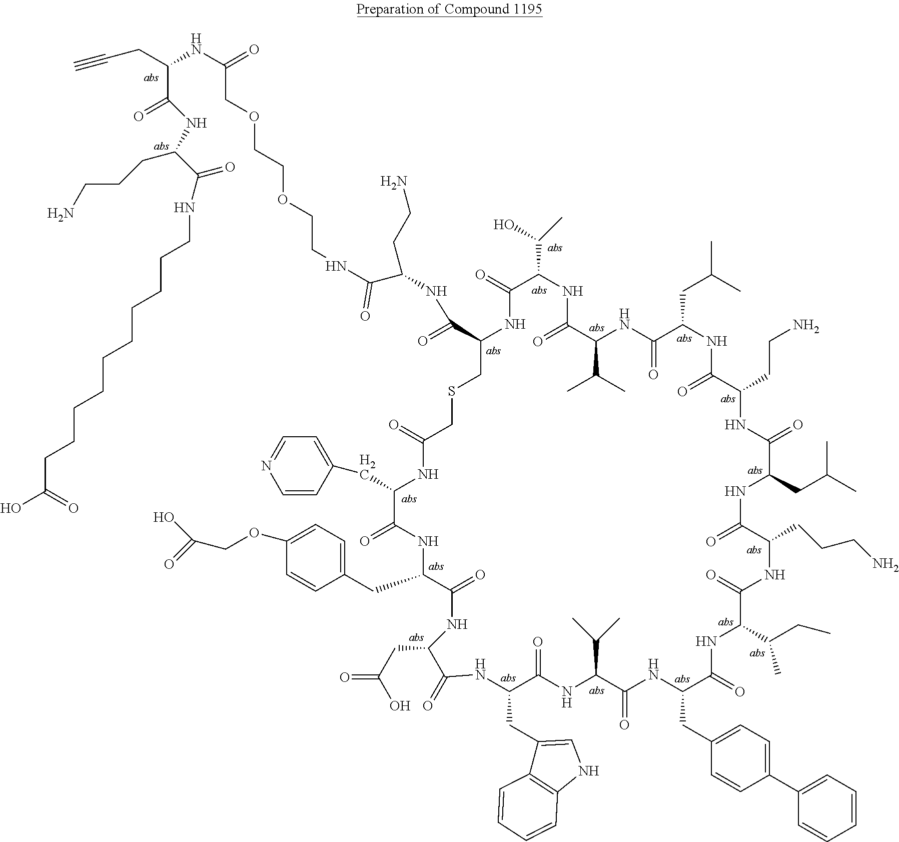 [SMILES: C#CC[C@H](NC(=O)COCCOCCNC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCN)C(=O)NCCCCCCCCCCC(=O)O]

Compound 1195 was prepared on a 50 μmol scale. The yield of the product was 7.8 mg, and its estimated purity by LCMS analysis was 86.3%. Analysis condition B: Retention time=1.54 min; ESI-MS(+) m/z [M+3H]³⁺: 849.4.

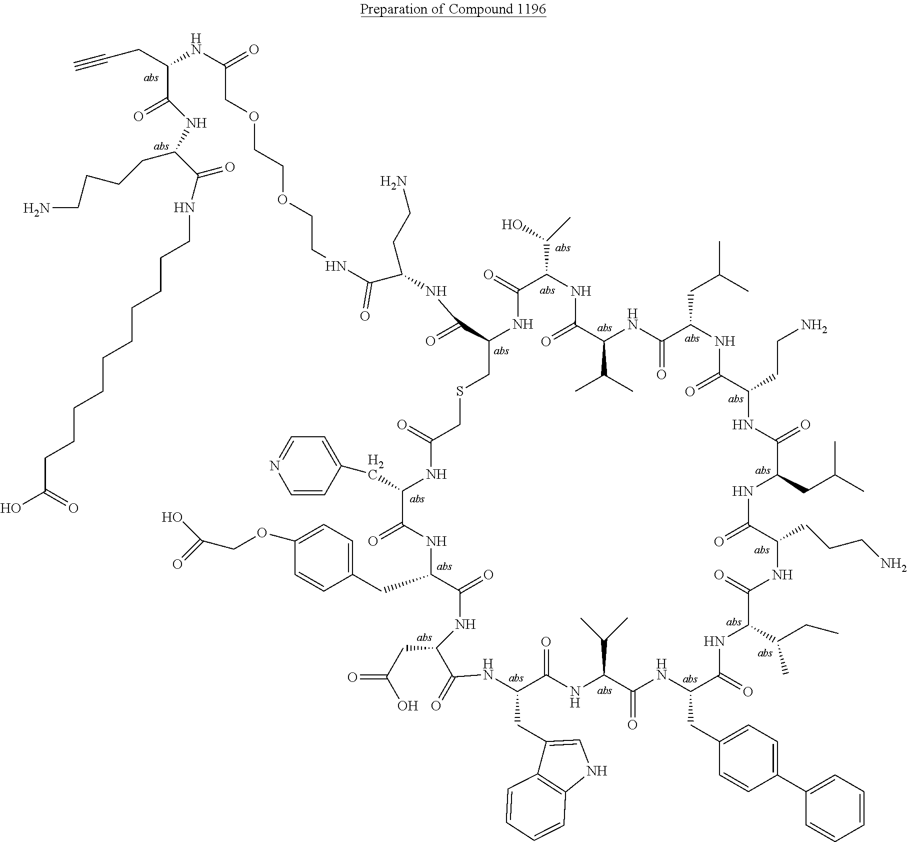 [SMILES: C#CC[C@H](NC(=O)COCCOCCNC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCCN)C(=O)NCCCCCCCCCCC(=O)O]

Compound 1196 was prepared on a 50 μmol scale. The yield of the product was 6.1 mg, and its estimated purity by LCMS analysis was 96.2%. Analysis condition B: Retention time=1.5 min; ESI-MS(+) m/z [M+2H]²⁺: 1280.9.

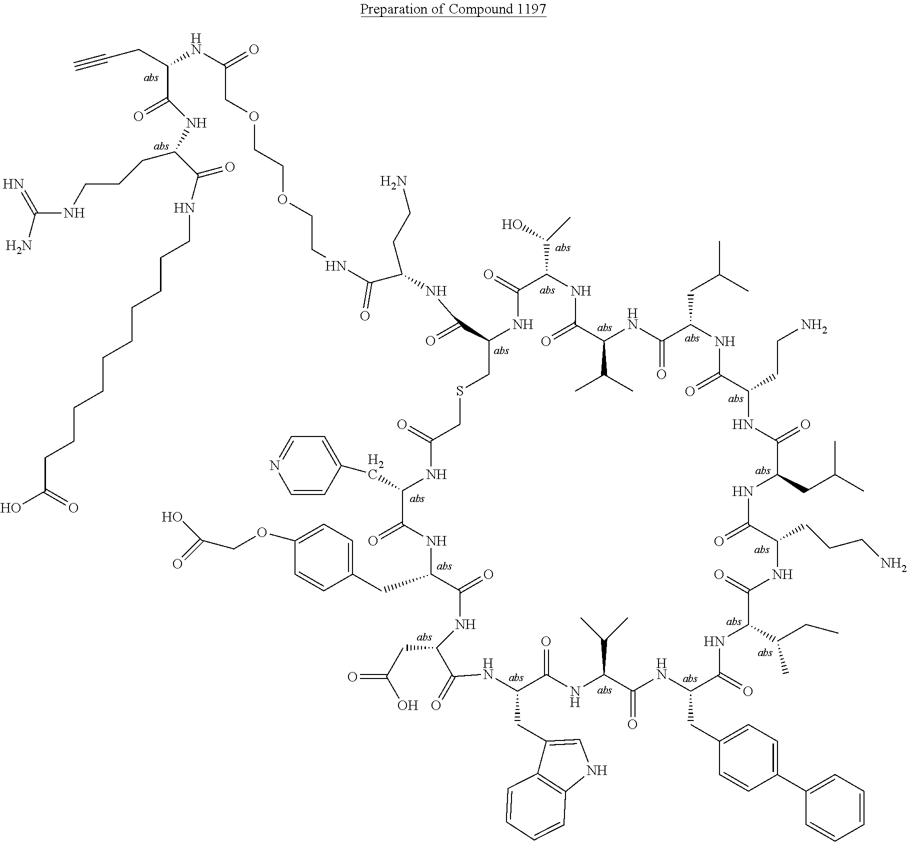 [SMILES: C#CC[C@H](NC(=O)COCCOCCNC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCNC(=N)N)C(=O)NCCCCCCCCCCC(=O)O]

Compound 1197 was prepared on a 50 μmol scale. The yield of the product was 12 mg, and its estimated purity by LCMS analysis was 78.7%. Analysis condition B: Retention time=1.55 min; ESI-MS(+) m/z [M+3H]³⁺: 863.2.

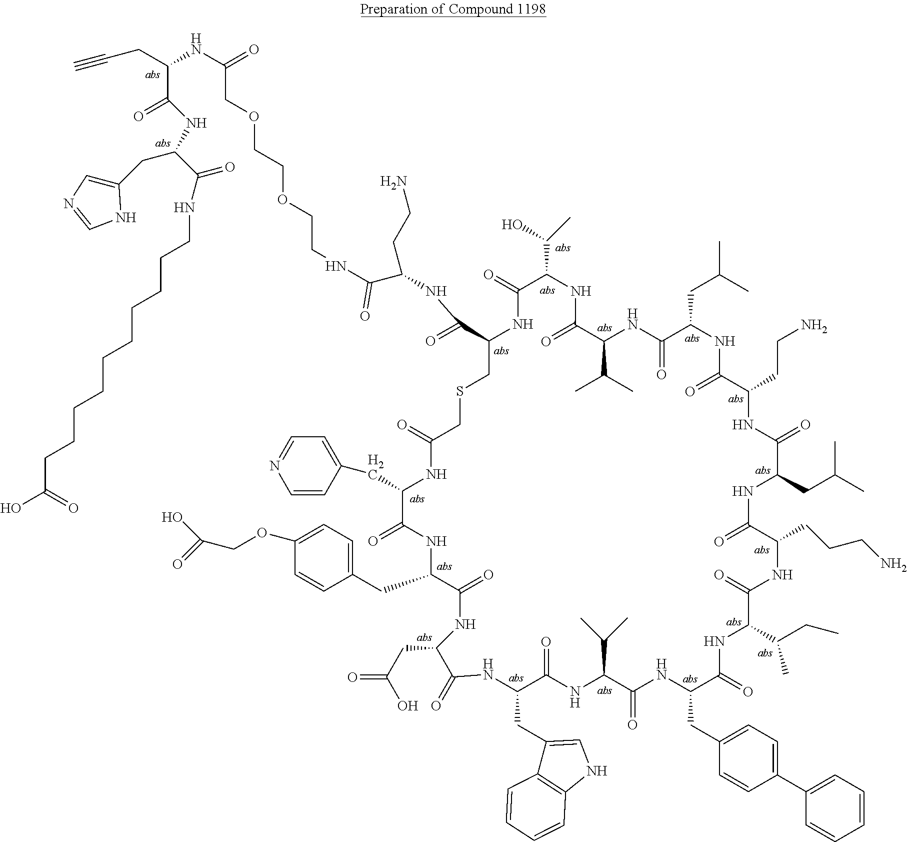 [SMILES: C#CC[C@H](NC(=O)COCCOCCNC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](Cc1cnc[nH]1)C(=O)NCCCCCCCCCCC(=O)O]

Compound 1198 was prepared on a 50 μmol scale. The yield of the product was 7.2 mg, and its estimated purity by LCMS analysis was 89%. Analysis condition A: Retention time=1.51 min; ESI-MS(+) m/z [M+3H]³⁺: 856.9.

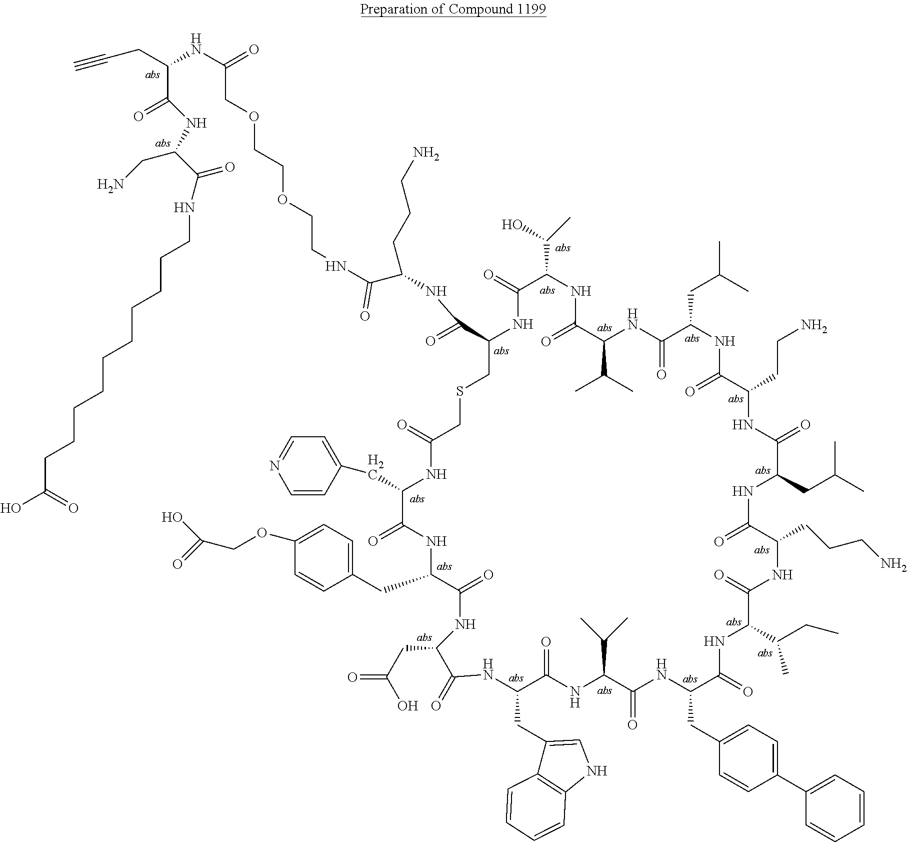 [SMILES: C#CC[C@H](NC(=O)COCCOCCNC(=O)[C@H](CCCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CN)C(=O)NCCCCCCCCCCC(=O)O]

Compound 1199 was prepared on a 50 μmol scale. The yield of the product was 6.2 mg, and its estimated purity by LCMS analysis was 93.5%. Analysis condition A: Retention time=1.52 min; ESI-MS(+) m/z [M+3H]³⁺: 844.1.

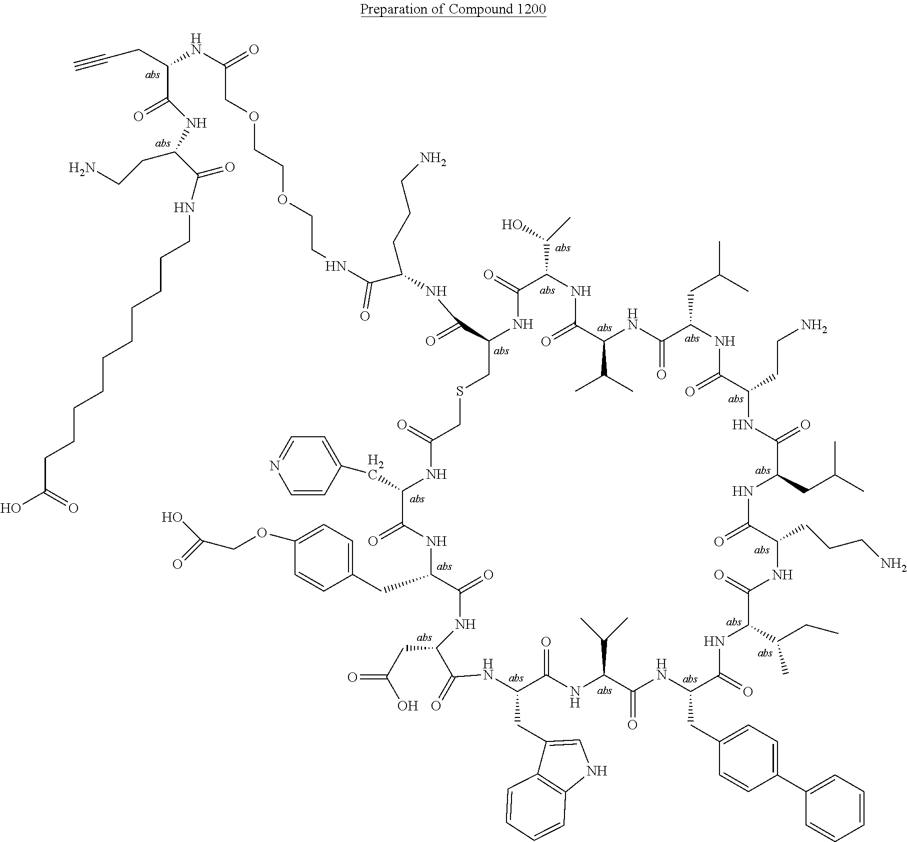 [SMILES: C#CC[C@H](NC(=O)COCCOCCNC(=O)[C@H](CCCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCN)C(=O)NCCCCCCCCCCC(=O)O]

Compound 1200 was prepared on a 50 μmol scale. The yield of the product was 9.9 mg, and its estimated purity by LCMS analysis was 84.2%. Analysis condition A: Retention time=1.47 min; ESI-MS(+) m/z [M+2H]²⁺: 1273.6.

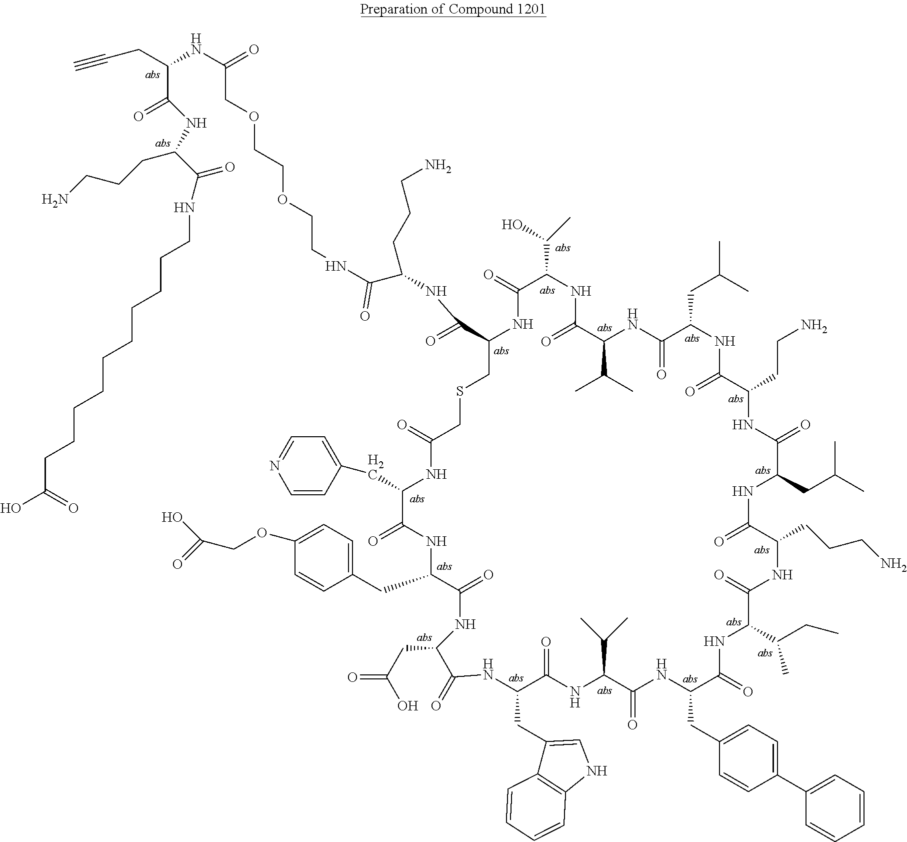 [SMILES: C#CC[C@H](NC(=O)COCCOCCNC(=O)[C@H](CCCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCN)C(=O)NCCCCCCCCCCC(=O)O]

Compound 1201 was prepared on a 50 μmol scale. The yield of the product was 8.1 mg, and its estimated purity by LCMS analysis was 89.1%. Analysis condition B: Retention time=1.46 min; ESI-MS(+) m/z [M+3H]³⁺: 854.1.

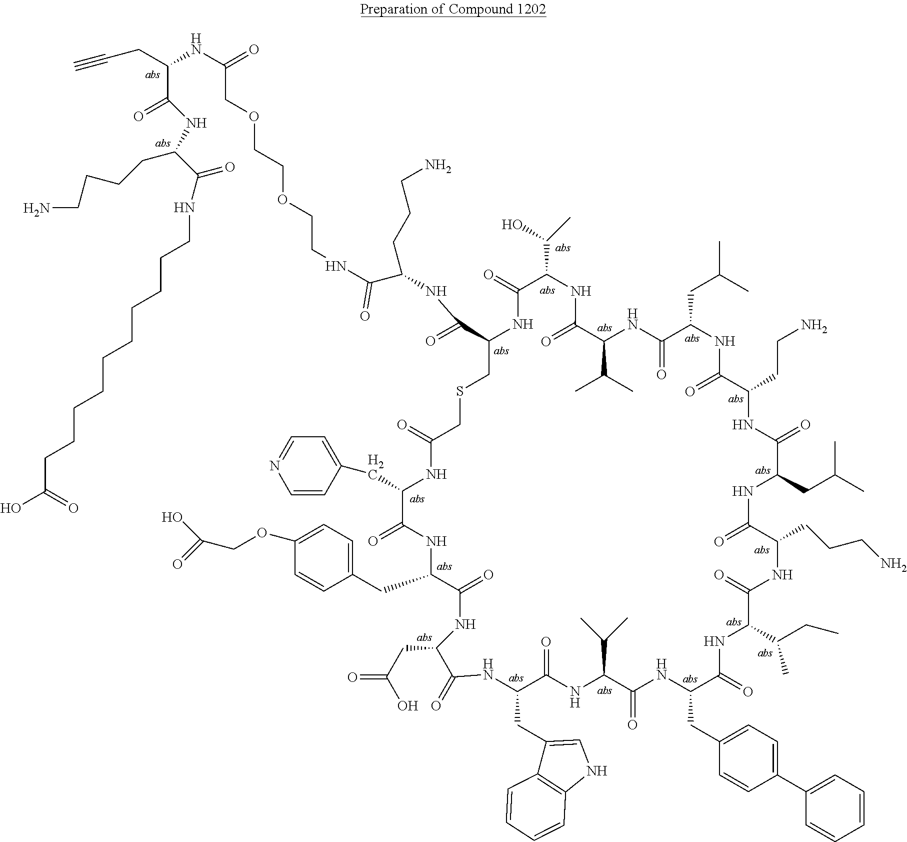 [SMILES: C#CC[C@H](NC(=O)COCCOCCNC(=O)[C@H](CCCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCCN)C(=O)NCCCCCCCCCCC(=O)O]

Compound 1202 was prepared on a 50 μmol scale. The yield of the product was 10.1 mg, and its estimated purity by LCMS analysis was 88.2%. Analysis condition B: Retention time=1.53 min; ESI-MS(+) m/z [M+3H]³⁺: 859.1.

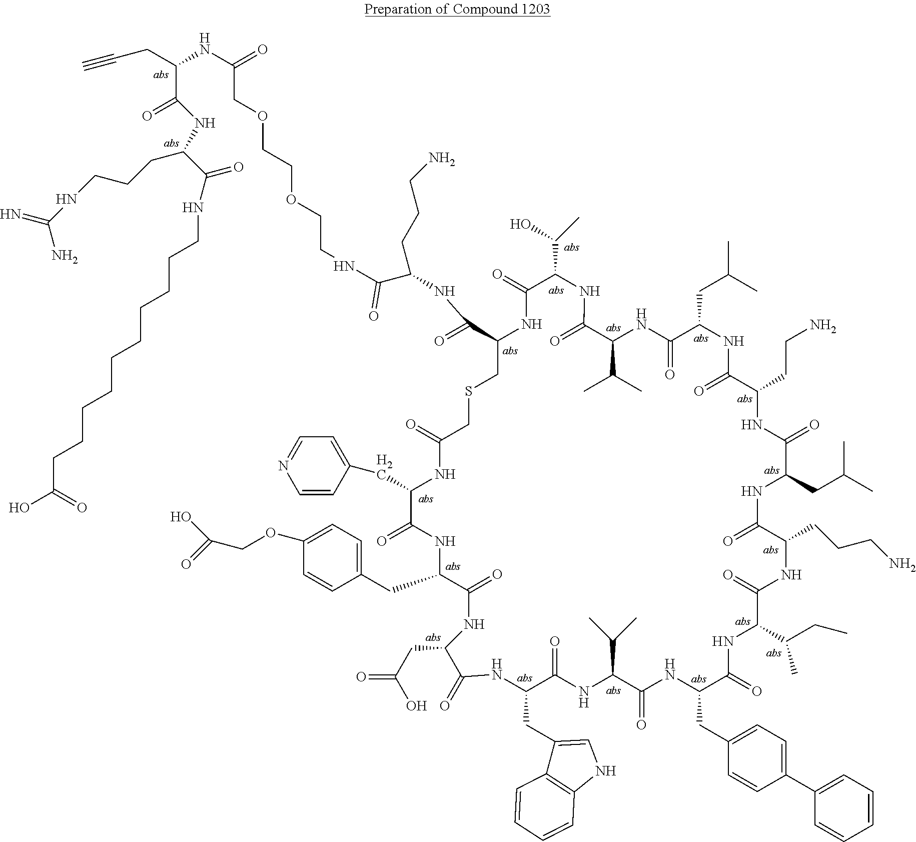 [SMILES: C#CC[C@H](NC(=O)COCCOCCNC(=O)[C@H](CCCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCNC(=N)N)C(=O)NCCCCCCCCCCC(=O)O]

Compound 1203 was prepared on a 50 μmol scale. The yield of the product was 8.9 mg, and its estimated purity by LCMS analysis was 94.4%. Analysis condition B: Retention time=1.53 min; ESI-MS(+) m/z [M+3H]³⁺: 868.2.

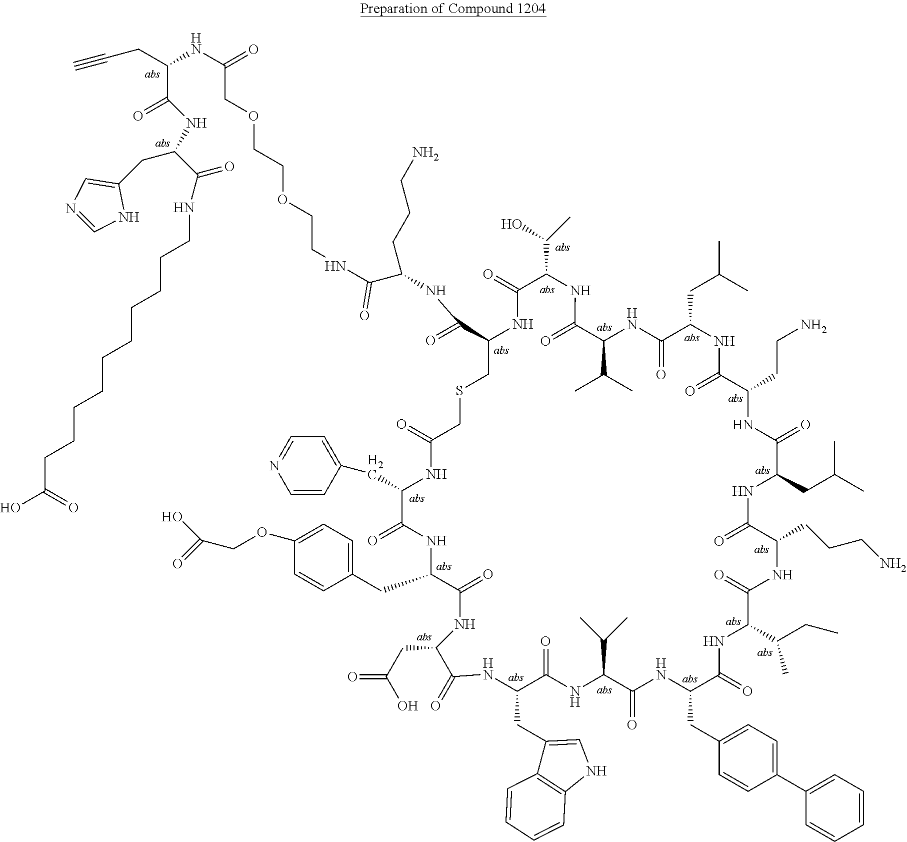 [SMILES: C#CC[C@H](NC(=O)COCCOCCNC(=O)[C@H](CCCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](Cc1cnc[nH]1)C(=O)NCCCCCCCCCCC(=O)O]

Compound 1204 was prepared on a 50 μmol scale. The yield of the product was 8.5 mg, and its estimated purity by LCMS analysis was 83.7%. Analysis condition B: Retention time=1.53 min; ESI-MS(+) m/z [M+3H]³⁺: 861.6.

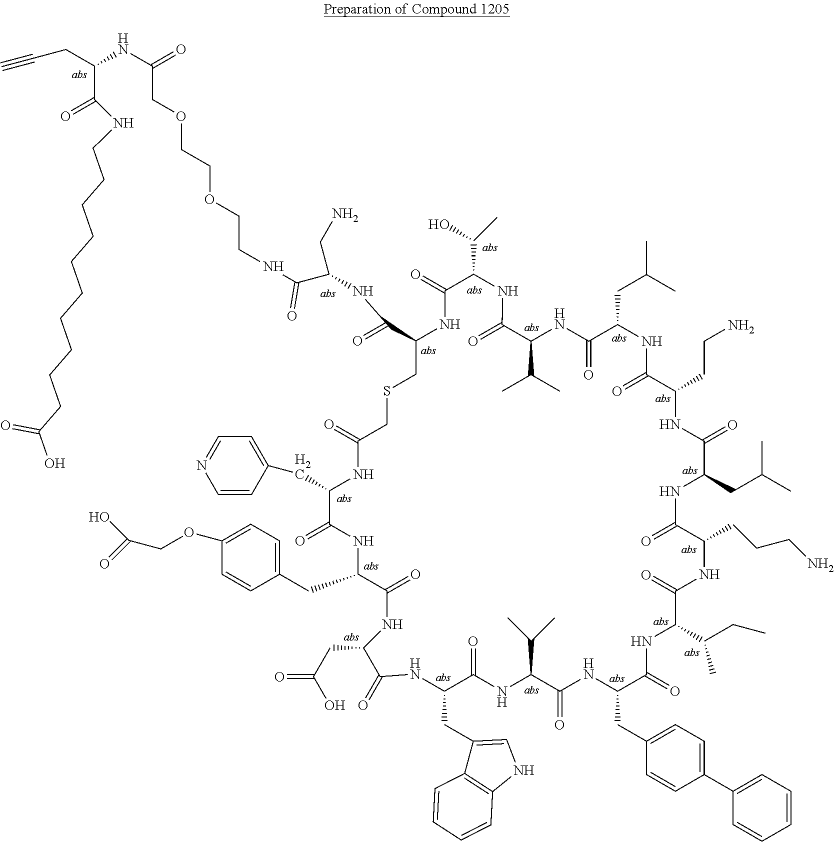 [SMILES: C#CC[C@H](NC(=O)COCCOCCNC(=O)[C@H](CN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)NCCCCCCCCCCC(=O)O]

Compound 1205 was prepared on a 50 μmol scale. The yield of the product was 3.5 mg, and its estimated purity by LCMS analysis was 83.6%. Analysis condition A: Retention time=1.62, 1.67 min; ESI-MS(+) m/z [M+2H]²⁺: 1209.93, 1209.92.

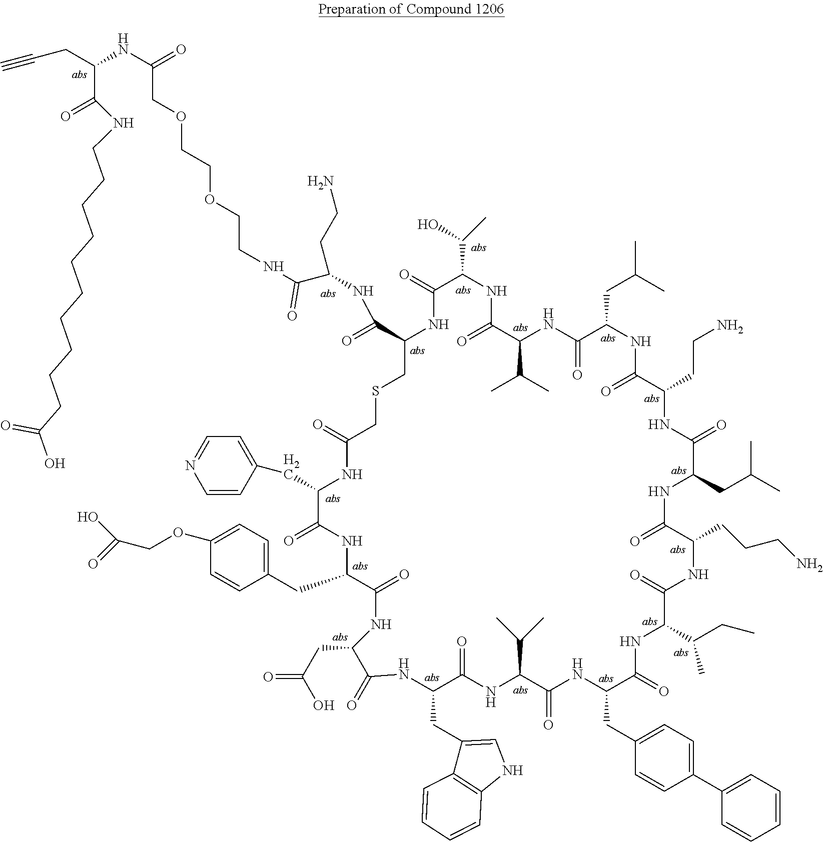 [SMILES: C#CC[C@H](NC(=O)COCCOCCNC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)NCCCCCCCCCCC(=O)O]

Compound 1206 was prepared on a 50 μmol scale. The yield of the product was 23.3 mg, and its estimated purity by LCMS analysis was 85%. Analysis condition A: Retention time=1.56 min; ESI-MS(+) m/z [M+3H]³⁺: 811.2.

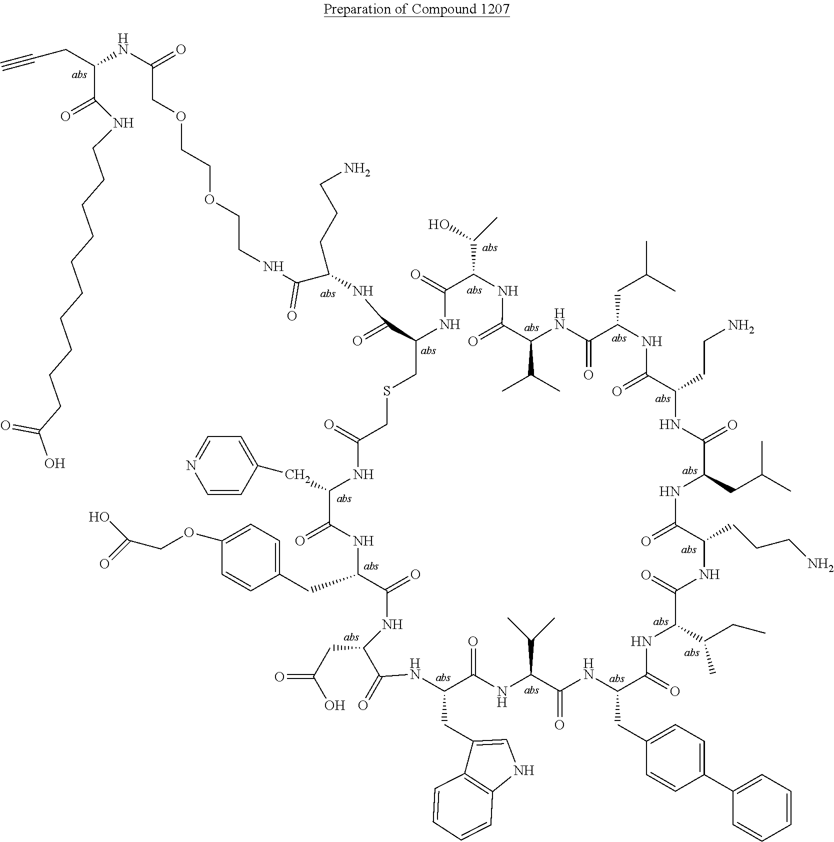 [SMILES: C#CC[C@H](NC(=O)COCCOCCNC(=O)[C@H](CCCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)NCCCCCCCCCCC(=O)O]

Compound 1207 was prepared on a 50 μmol scale. The yield of the product was 20.3 mg, and its estimated purity by LCMS analysis was 85.9%. Analysis condition B: Retention time=1.54 min; ESI-MS(+) m/z [M+2H]²⁺: 1224.

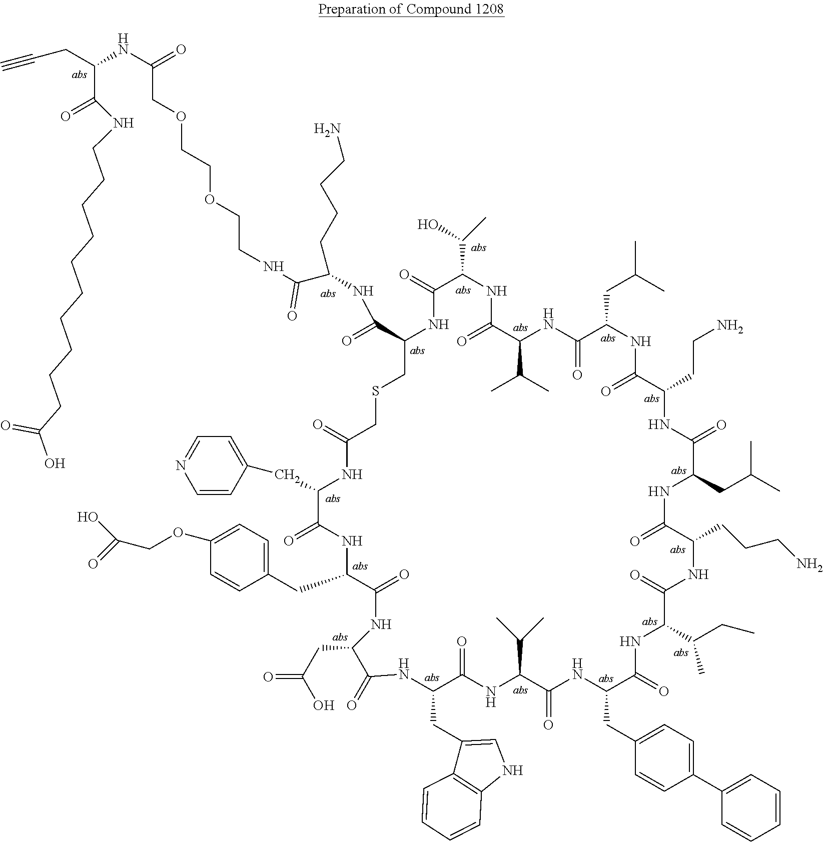 [SMILES: C#CC[C@H](NC(=O)COCCOCCNC(=O)[C@H](CCCCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)NCCCCCCCCCCC(=O)O]

Compound 1208 was prepared on a 50 μmol scale. The yield of the product was 17 mg, and its estimated purity by LCMS analysis was 87.6%. Analysis condition B: Retention time=1.59 min; ESI-MS(+) m/z [M+3H]³⁺: 821.

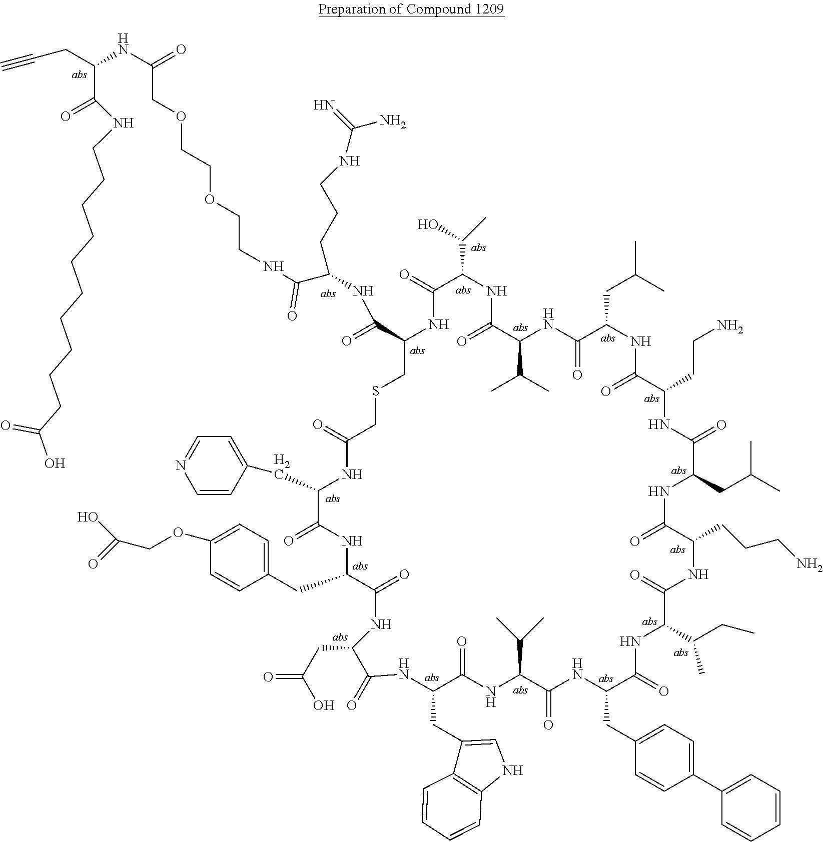 [SMILES: C#CC[C@H](NC(=O)COCCOCCNC(=O)[C@H](CCCNC(=N)N)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)NCCCCCCCCCCC(=O)O]

Compound 1209 was prepared on a 50 μmol scale. The yield of the product was 9.6 mg, and its estimated purity by LCMS analysis was 87.5%. Analysis condition B: Retention time=1.61 min; ESI-MS(+) m/z [M+3H]³⁺: 830.

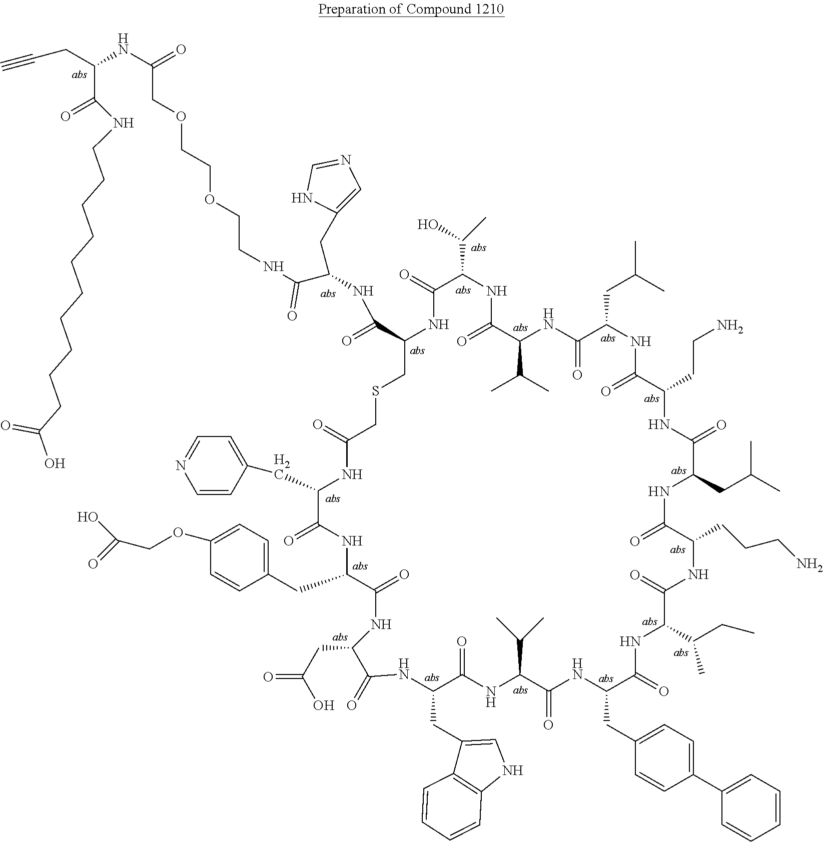 [SMILES: C#CC[C@H](NC(=O)COCCOCCNC(=O)[C@H](Cc1cnc[nH]1)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)NCCCCCCCCCCC(=O)O]

Compound 1210 was prepared on a 50 μmol scale. The yield of the product was 6.5 mg, and its estimated purity by LCMS analysis was 87.9%. Analysis condition A: Retention time=1.66, 1.72 min; ESI-MS(+) m/z [M+3H]³⁺: 823.1, 823.22.

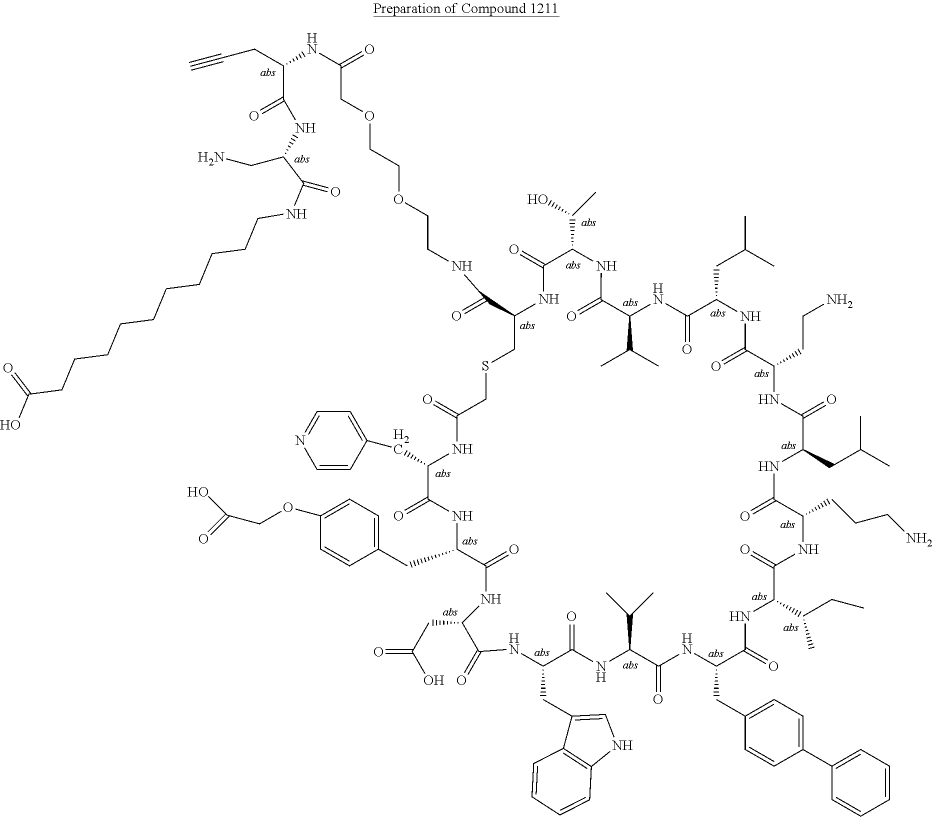 [SMILES: C#CC[C@H](NC(=O)COCCOCCNC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CN)C(=O)NCCCCCCCCCCC(=O)O]

Compound 1211 was prepared on a 50 μmol scale. The yield of the product was 10.9 mg, and its estimated purity by LCMS analysis was 80.8%. Analysis condition B: Retention time=1.61 min; ESI-MS(+) m/z [M+3H]³⁺: 807.

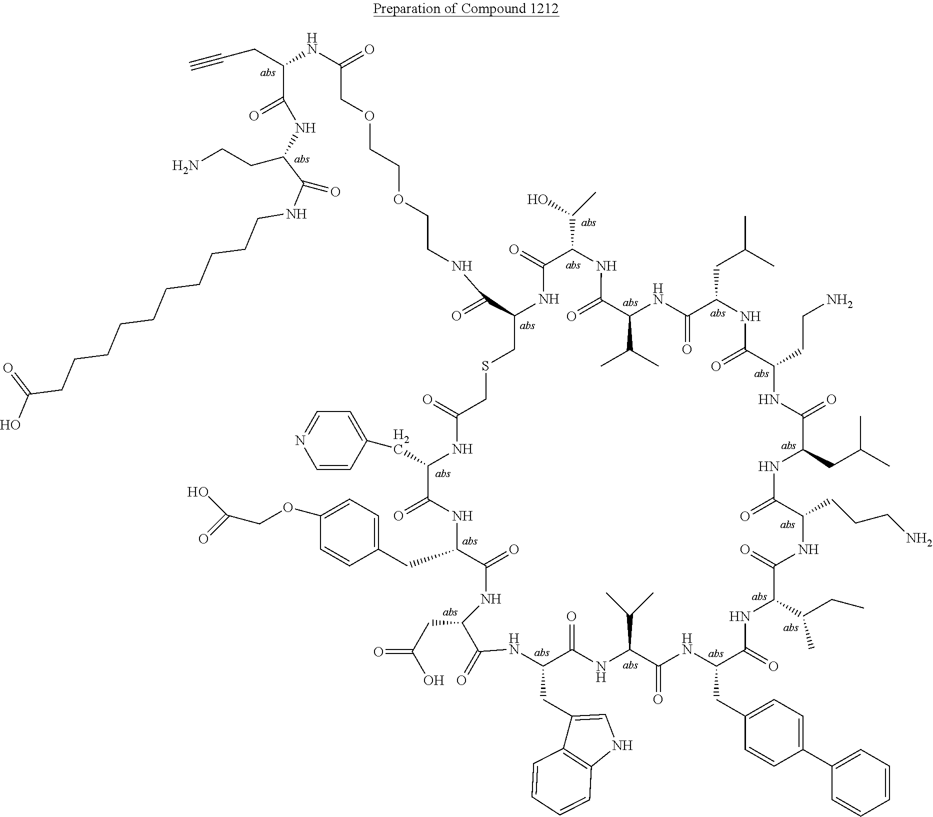 [SMILES: C#CC[C@H](NC(=O)COCCOCCNC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCN)C(=O)NCCCCCCCCCCC(=O)O]

Compound 1212 was prepared on a 50 μmol scale. The yield of the product was 7.6 mg, and its estimated purity by LCMS analysis was 85.9%. Analysis condition A: Retention time=1.63 min; ESI-MS(+) m/z [M+3H]³⁺: 811.1.

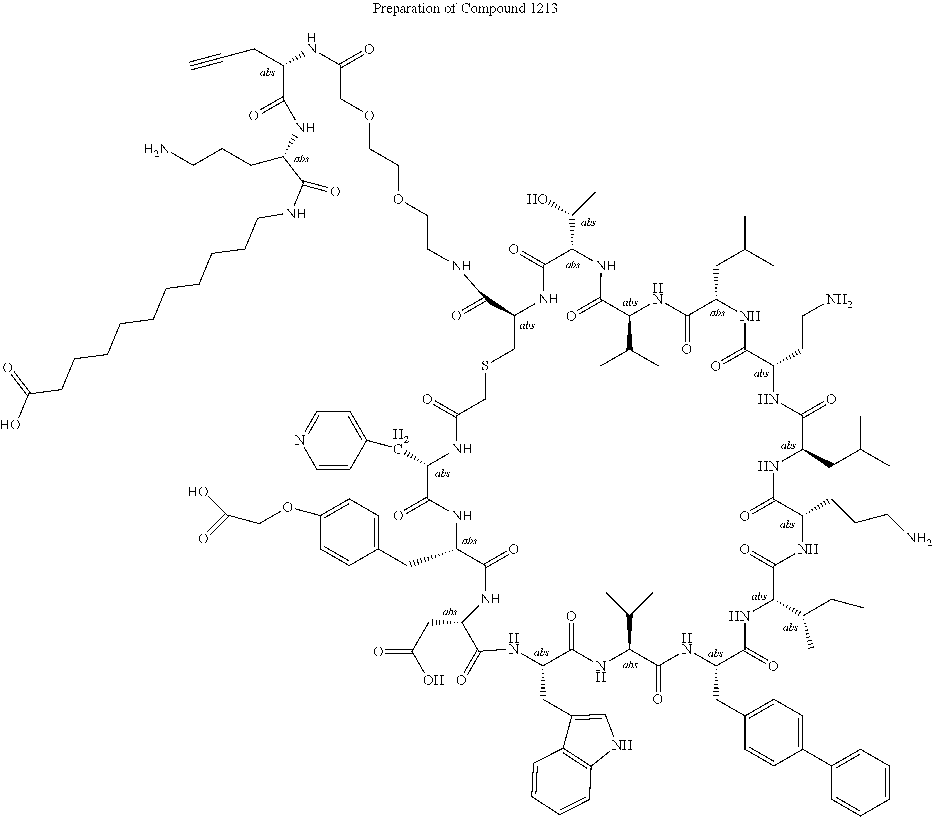 [SMILES: C#CC[C@H](NC(=O)COCCOCCNC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCN)C(=O)NCCCCCCCCCCC(=O)O]

Compound 1213 was prepared on a 50 μmol scale. The yield of the product was 7.7 mg, and its estimated purity by LCMS analysis was 84.5%. Analysis condition A: Retention time=1.62 min; ESI-MS(+) m/z [M+3H]³⁺: 816.1.

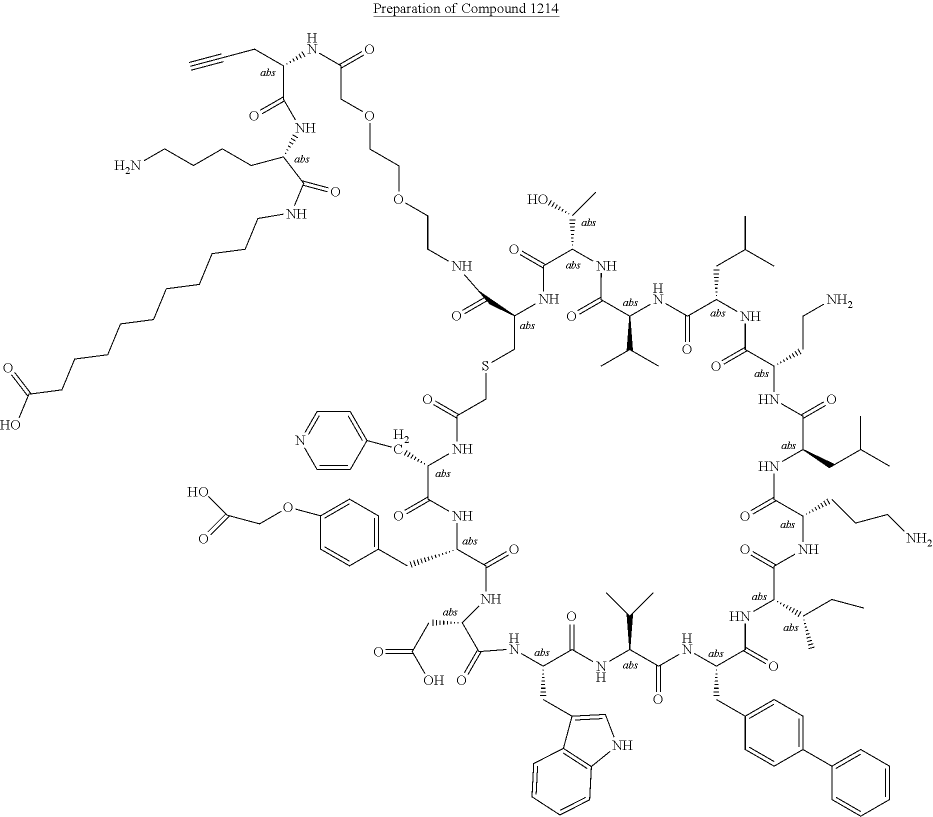 [SMILES: C#CC[C@H](NC(=O)COCCOCCNC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCCN)C(=O)NCCCCCCCCCCC(=O)O]

Compound 1214 was prepared on a 50 μmol scale. The yield of the product was 12.4 mg, and its estimated purity by LCMS analysis was 95.1%. Analysis condition B: Retention time=1.55 min; ESI-MS(+) m/z [M+2H]²⁺: 1230.9.

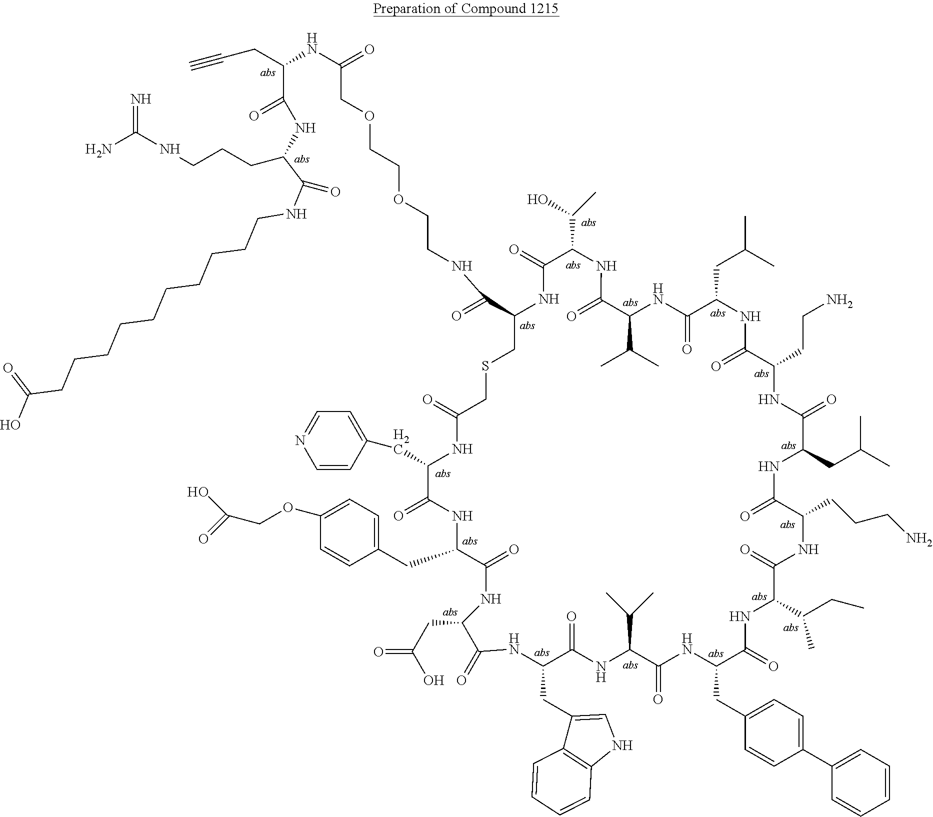 [SMILES: C#CC[C@H](NC(=O)COCCOCCNC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](CCCNC(=N)N)C(=O)NCCCCCCCCCCC(=O)O]

Compound 1215 was prepared on a 50 μmol scale. The yield of the product was 11.9 mg, and its estimated purity by LCMS analysis was 90.3%. Analysis condition B: Retention time=1.55 min; ESI-MS(+) m/z [M+2H]²⁺: 1245.

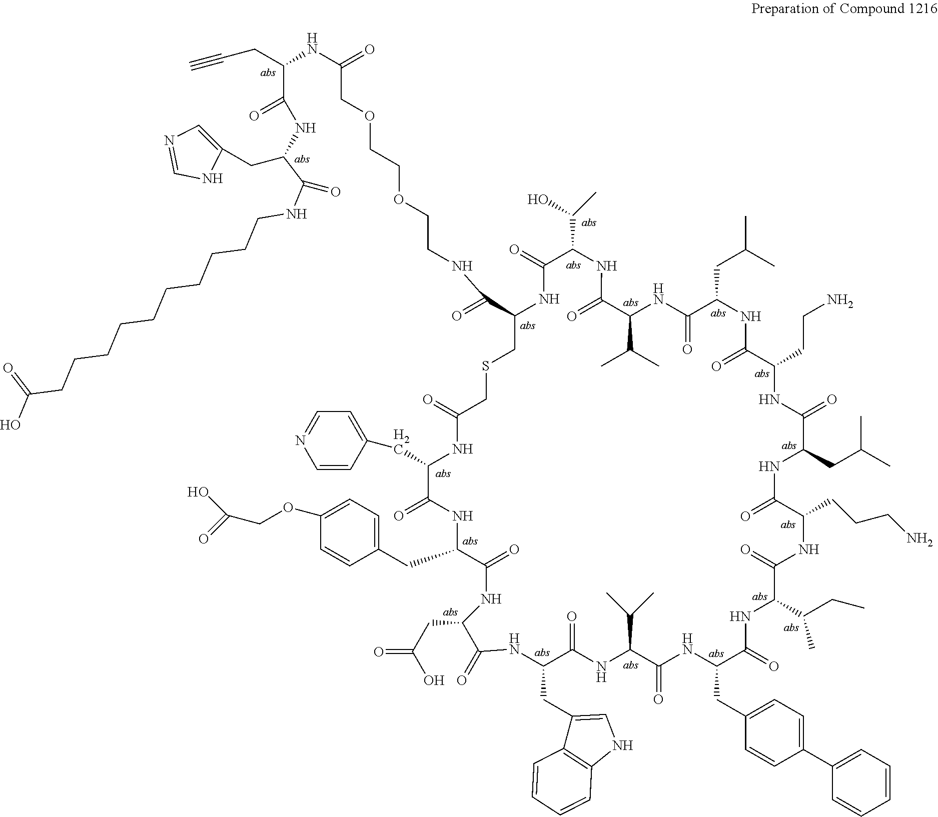 [SMILES: C#CC[C@H](NC(=O)COCCOCCNC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)N[C@@H](Cc1cnc[nH]1)C(=O)NCCCCCCCCCCC(=O)O]

Compound 1216 was prepared on a 50 μmol scale. The yield of the product was 10.2 mg, and its estimated purity by LCMS analysis was 74.9%. Analysis condition A: Retention time=1.58, 1.66 min; ESI-MS(+) m/z [M+2H]²⁺: 1234.9, 1235.11.

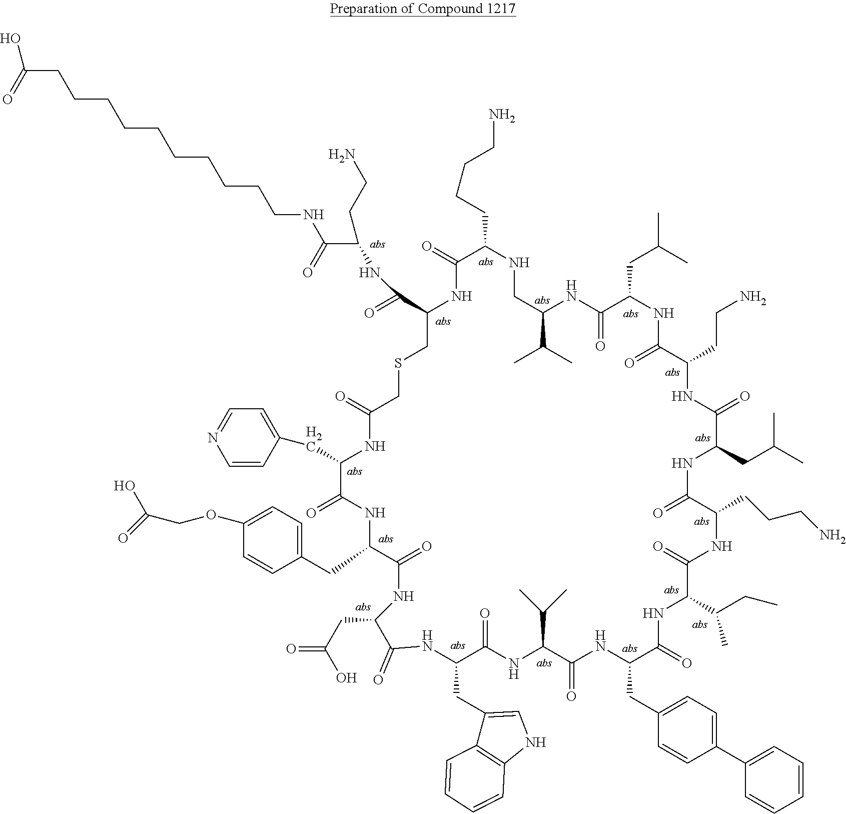 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H](CCCCN)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1217 was prepared on a 50 μmol scale. The yield of the product was 1.8 mg, and its estimated purity by LCMS analysis was 84.4%. Analysis condition B: Retention time=1.45 min; ESI-MS(+) m/z [M+2H]²⁺: 1110.2.

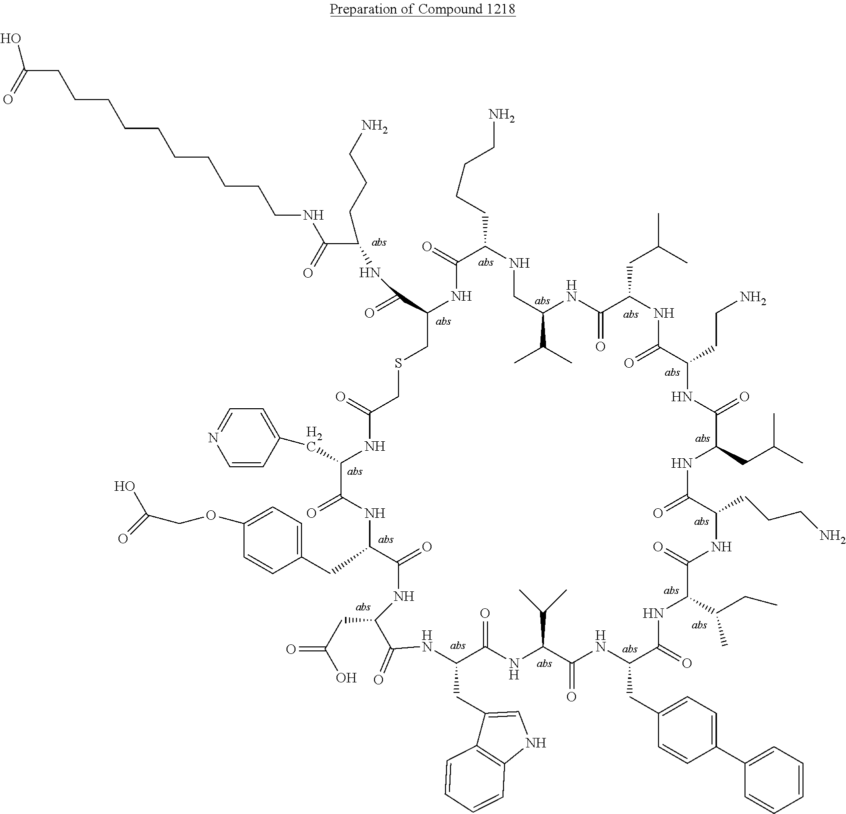 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCCCCCCCCCC(=O)O)NC(=O)[C@H](CCCCN)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1218 was prepared on a 50 μmol scale. The yield of the product was 2.4 mg, and its estimated purity by LCMS analysis was 100%. Analysis condition A: Retention time=1.49 min; ESI-MS(+) m/z [M+2H]²⁺: 1117.1.

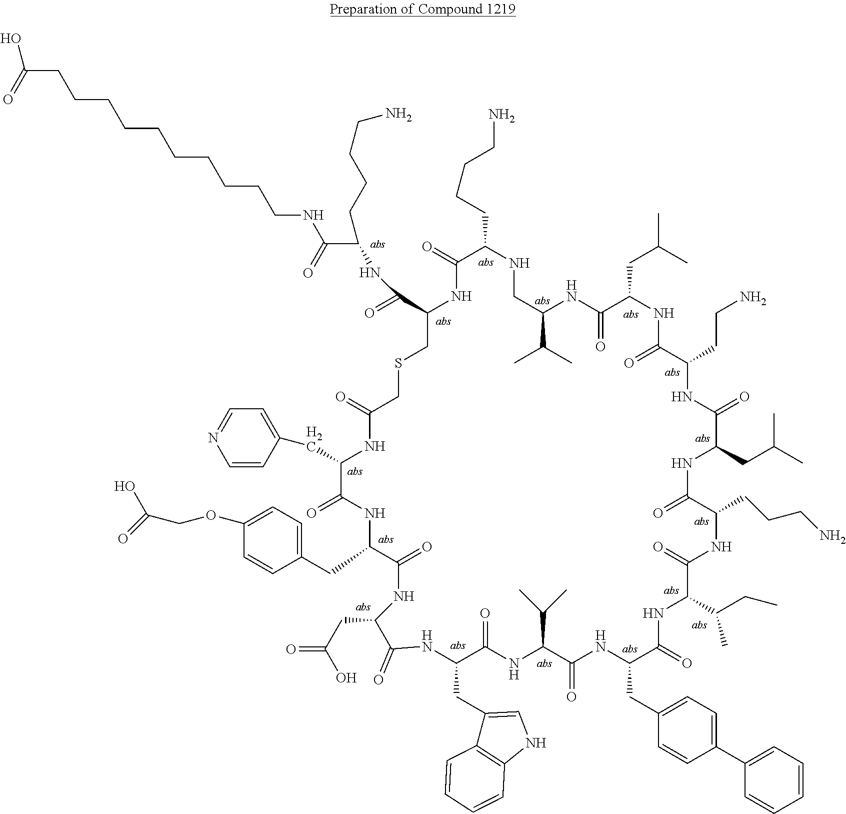 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCCN)C(=O)NCCCCCCCCCCC(=O)O)NC(=O)[C@H](CCCCN)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1219 was prepared on a 50 μmol scale. The yield of the product was 4.9 mg, and its estimated purity by LCMS analysis was 90.5%. Analysis condition B: Retention time=1.3 min; ESI-MS(+) m/z [M+2H]²⁺: 1124.1.

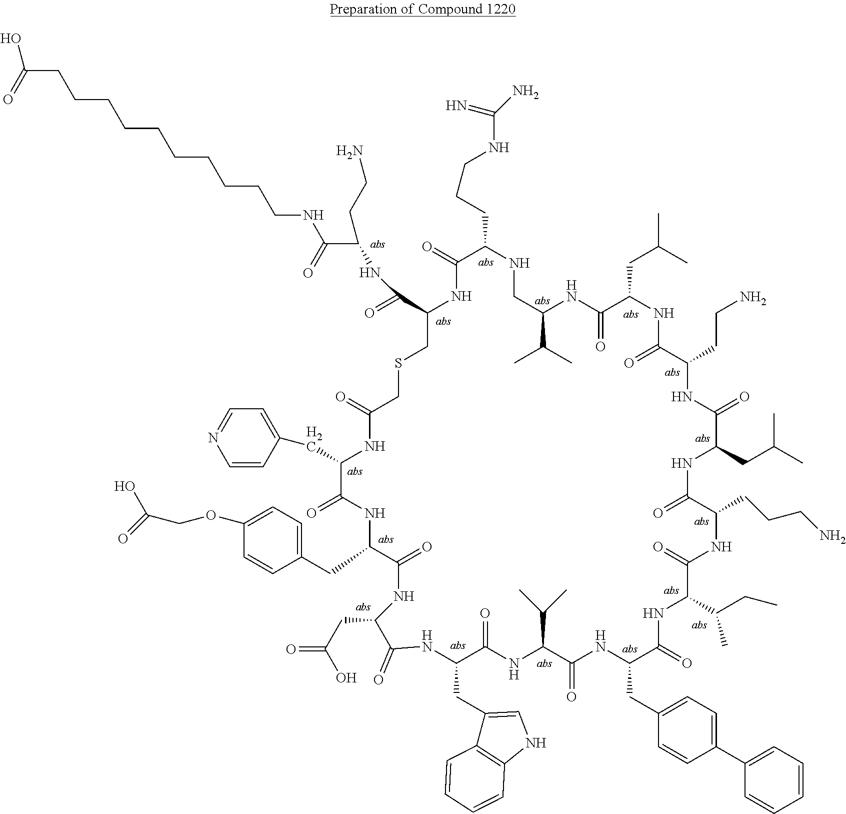 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H](CCCNC(=N)N)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1220 was prepared on a 50 μmol scale. The yield of the product was 6.9 mg, and its estimated purity by LCMS analysis was 86.6%. Analysis condition B: Retention time=1.43 min; ESI-MS(+) m/z [M+2H]²⁺: 1124.5.

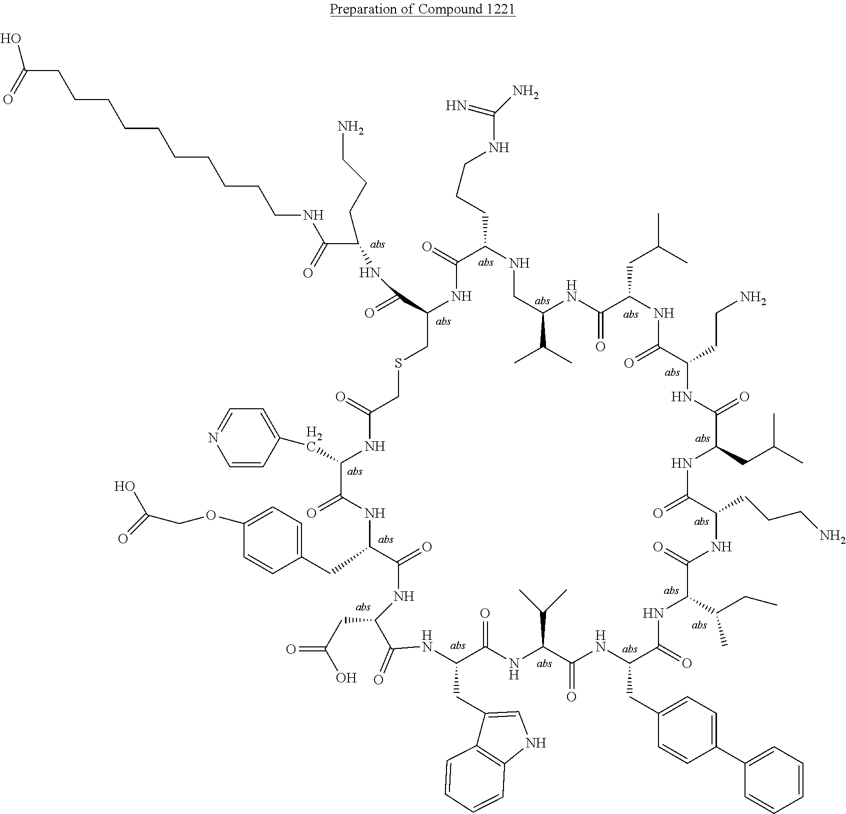 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCCCCCCCCCC(=O)O)NC(=O)[C@H](CCCNC(=N)N)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1221 was prepared on a 50 μmol scale. The yield of the product was 2.6 mg, and its estimated purity by LCMS analysis was 93.3%. Analysis condition B: Retention time=1.43 min; ESI-MS(+) m/z [M+2H]²⁺: 1131.1.

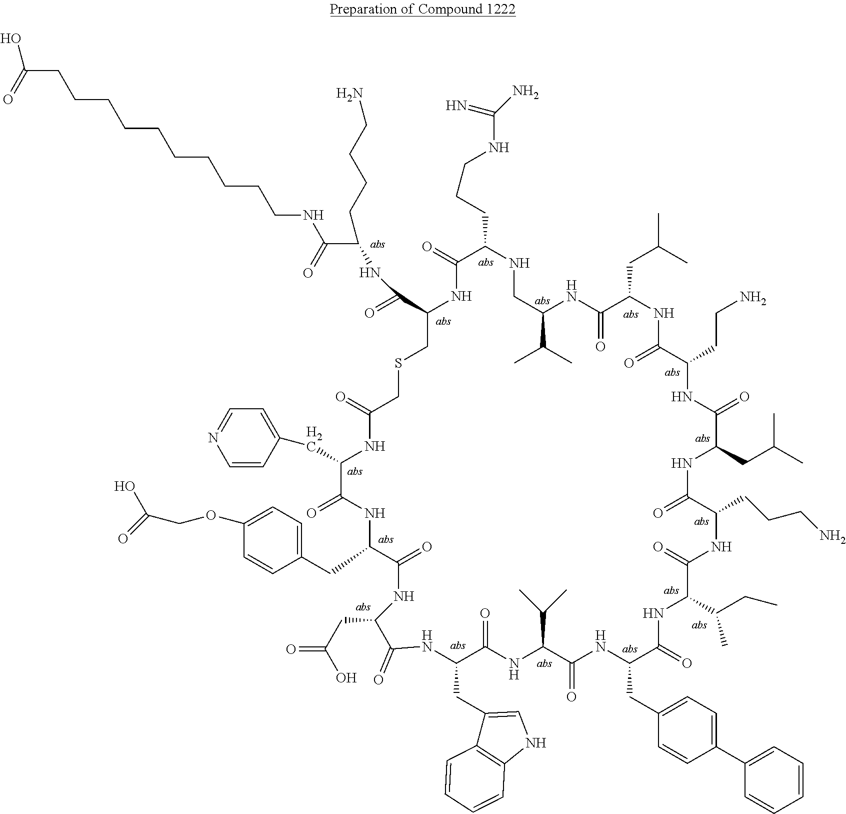 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCCN)C(=O)NCCCCCCCCCCC(=O)O)NC(=O)[C@H](CCCNC(=N)N)NC[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1222 was prepared on a 50 μmol scale. The yield of the product was 3.7 mg, and its estimated purity by LCMS analysis was 91.3%. Analysis condition B: Retention time=1.42 min; ESI-MS(+) m/z [M+2H]²⁺: 1138.2.

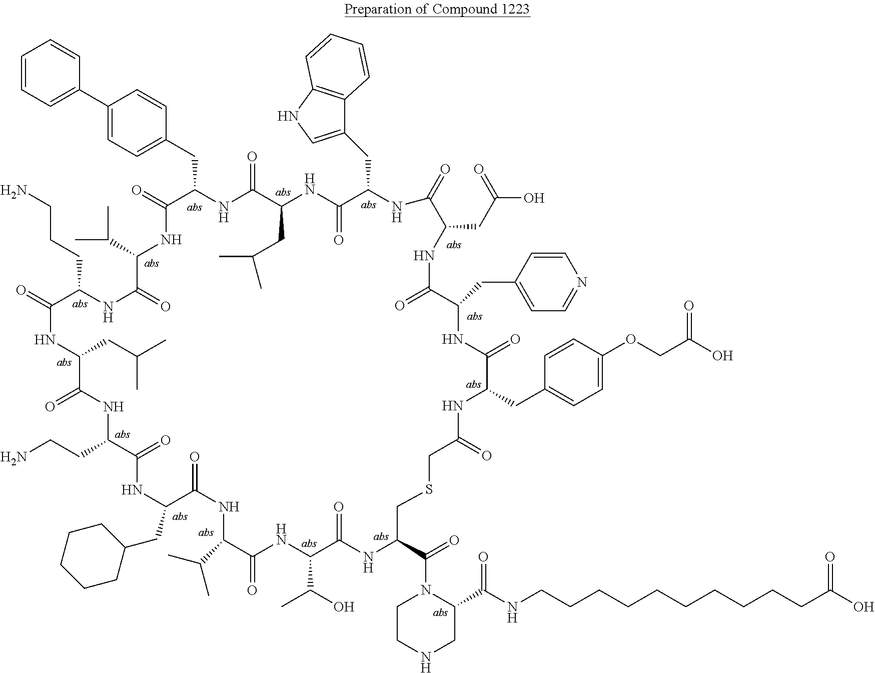 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCNC[C@H]2C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1223 was prepared on a 50 μmol scale. The yield of the product was 15.2 mg, and its estimated purity by LCMS analysis was 100%. Analysis condition A: Retention time=1.97 min; ESI-MS(+) m/z [M+2H]²⁺: 1122.9.

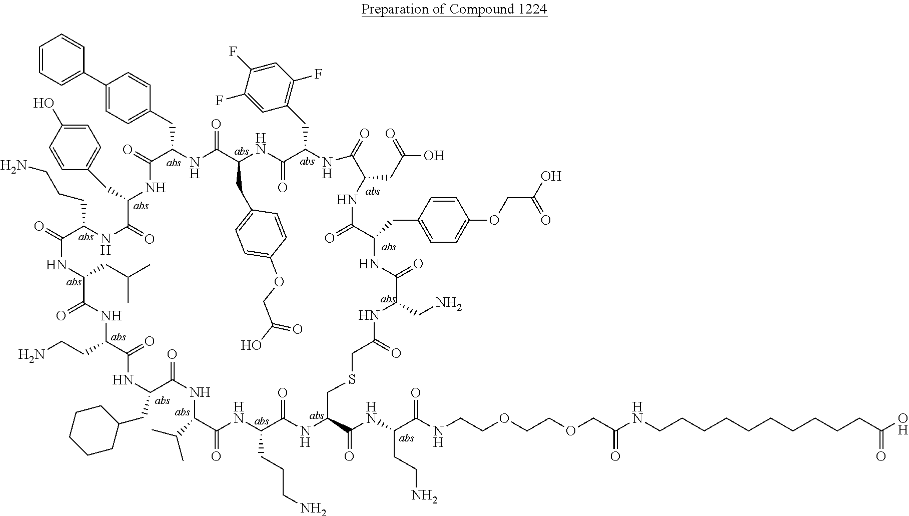 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2cc(F)c(F)cc2F)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1224 was prepared on a 50 μmol scale. The yield of the product was 7.5 mg, and its estimated purity by LCMS analysis was 92.7%. Analysis condition B: Retention time=1.6 min; ESI-MS(+) m/z [M+3H]³⁺: 838.8.

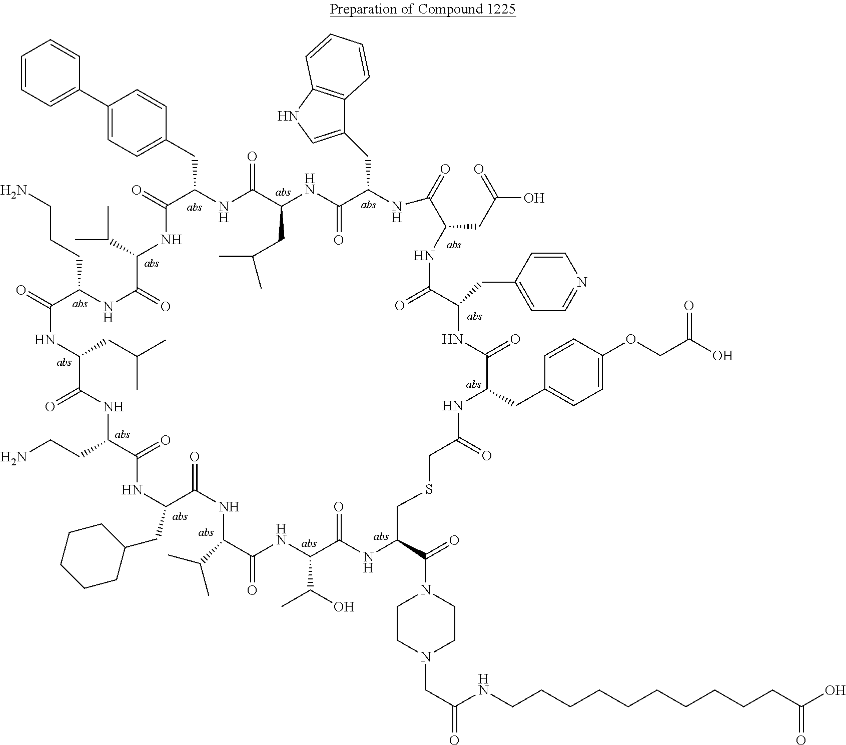 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCN(CC(=O)NCCCCCCCCCCC(=O)O)CC2)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1225 was prepared on a 50 μmol scale. The yield of the product was 10.5 mg, and its estimated purity by LCMS analysis was 89.1%. Analysis condition B: Retention time=1.65 min; ESI-MS(+) m/z [M+2H]²⁺: 1129.3.

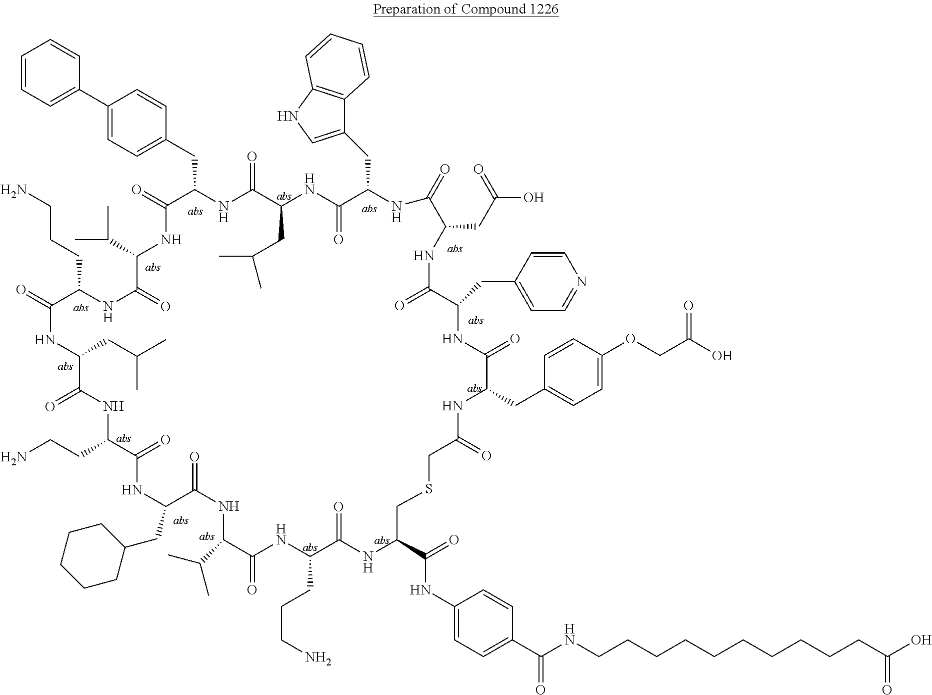 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2ccc(C(=O)NCCCCCCCCCCC(=O)O)cc2)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1226 was prepared on a 50 μmol scale. The yield of the product was 6.1 mg, and its estimated purity by LCMS analysis was 90.7%. Analysis condition A: Retention time=1.8 min; ESI-MS(+) m/z [M+2H]²⁺: 1132.3.

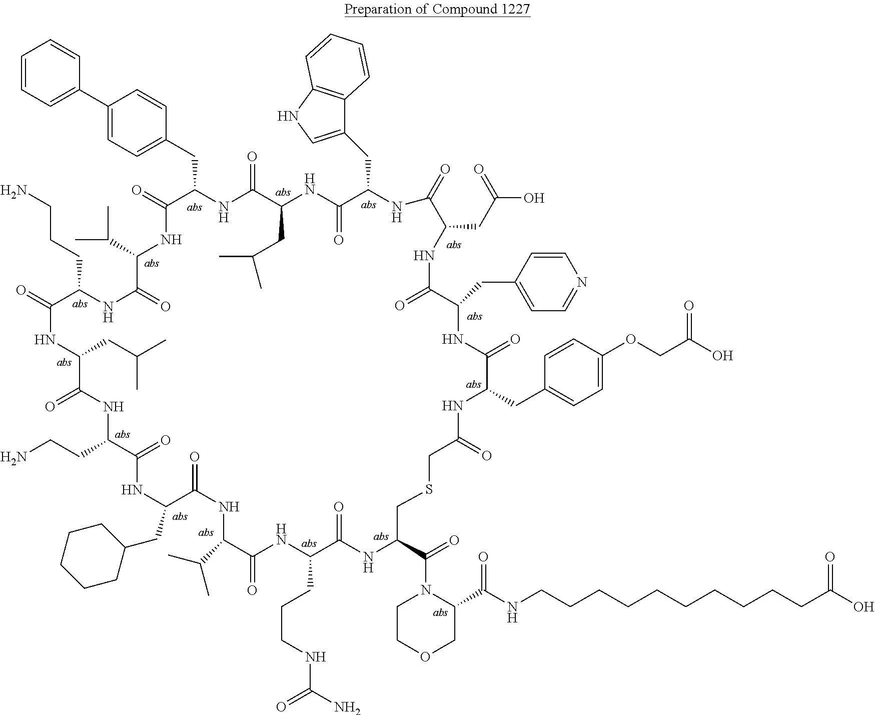 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCOC[C@H]2C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1227 was prepared on a 50 μmol scale. The yield of the product was 4.6 mg, and its estimated purity by LCMS analysis was 95.6%. Analysis condition A: Retention time=1.8 min; ESI-MS(+) m/z [M+2H]²⁺: 1151.2.

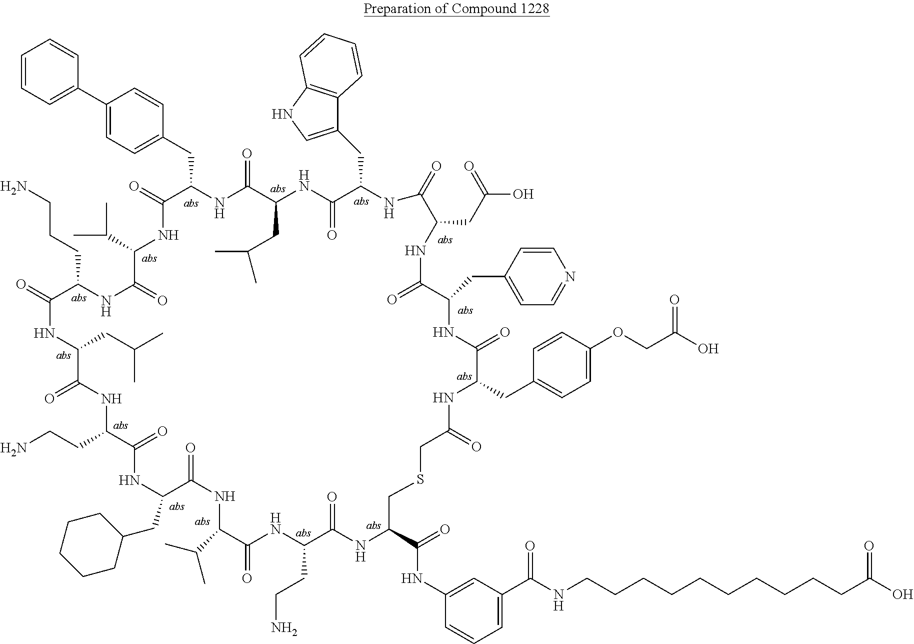 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2cccc(C(=O)NCCCCCCCCCCC(=O)O)c2)NC(=O)[C@H](CCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1228 was prepared on a 50 μmol scale. The yield of the product was 0.8 mg, and its estimated purity by LCMS analysis was 97.6%. Analysis condition A: Retention time=1.88, 1.91 min; ESI-MS(+) m/z [M+2H]²⁺: 1125.22, 1125.21.

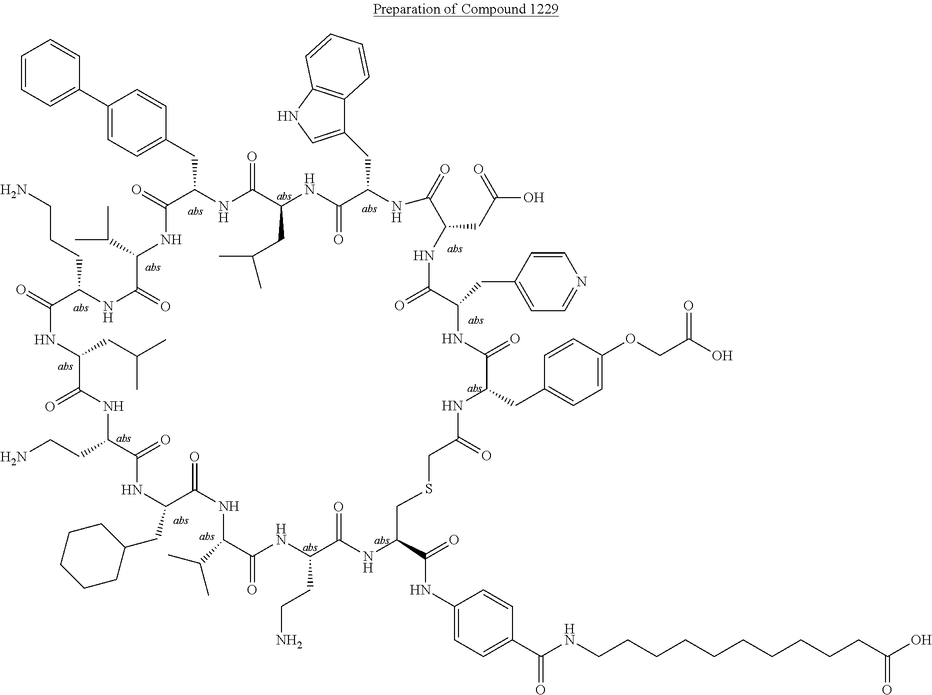 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2ccc(C(=O)NCCCCCCCCCCC(=O)O)cc2)NC(=O)[C@H](CCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1229 was prepared on a 50 μmol scale. The yield of the product was 8 mg, and its estimated purity by LCMS analysis was 88.8%. Analysis condition B: Retention time=1.55 min; ESI-MS(+) m/z [M+2H]²⁺: 1126.

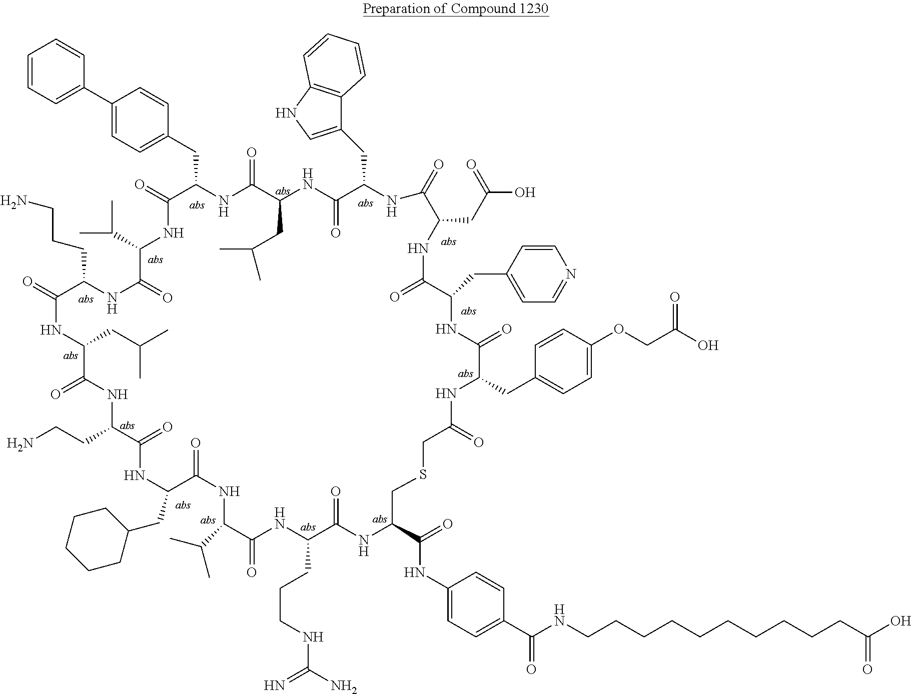 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2ccc(C(=O)NCCCCCCCCCCC(=O)O)cc2)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1230 was prepared on a 50 μmol scale. The yield of the product was 1.6 mg, and its estimated purity by LCMS analysis was 100%. Analysis condition A: Retention time=1.79 min; ESI-MS(+) m/z [M+3H]³⁺: 769.1.

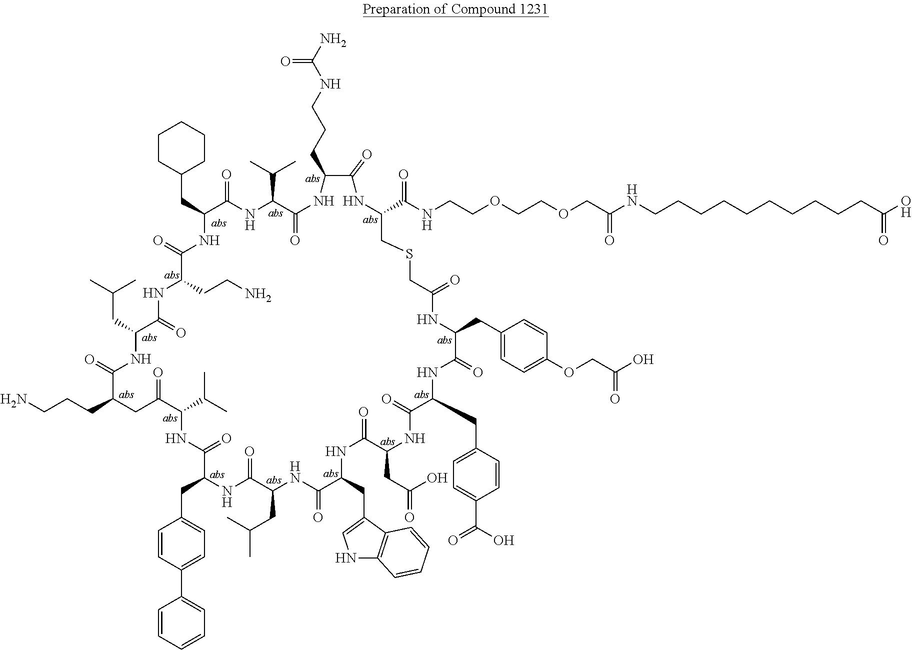 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)CC(=O)[C@H](C(C)C)NC(=O)[C@H](Cc2ccc(-c3ccccc3)cc2)NC1=O]

Compound 1231 was prepared on a 50 μmol scale. The yield of the product was 2.7 mg, and its estimated purity by LCMS analysis was 95.3%. Analysis condition A: Retention time=1.66 min; ESI-MS(+) m/z [M+2H]²⁺: 1188.4.

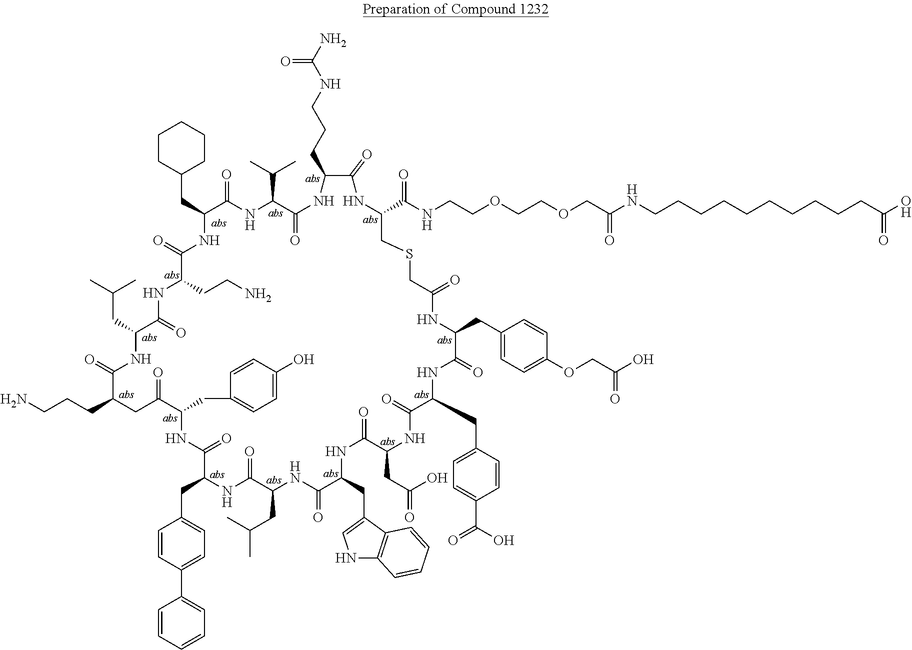 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)CC(=O)[C@H](Cc2ccc(O)cc2)NC(=O)[C@H](Cc2ccc(-c3ccccc3)cc2)NC1=O]

Compound 1232 was prepared on a 50 μmol scale. The yield of the product was 15.4 mg, and its estimated purity by LCMS analysis was 100%. Analysis condition A: Retention time=1.57 min; ESI-MS(+) m/z [M+2H]²⁺: 1220.2.

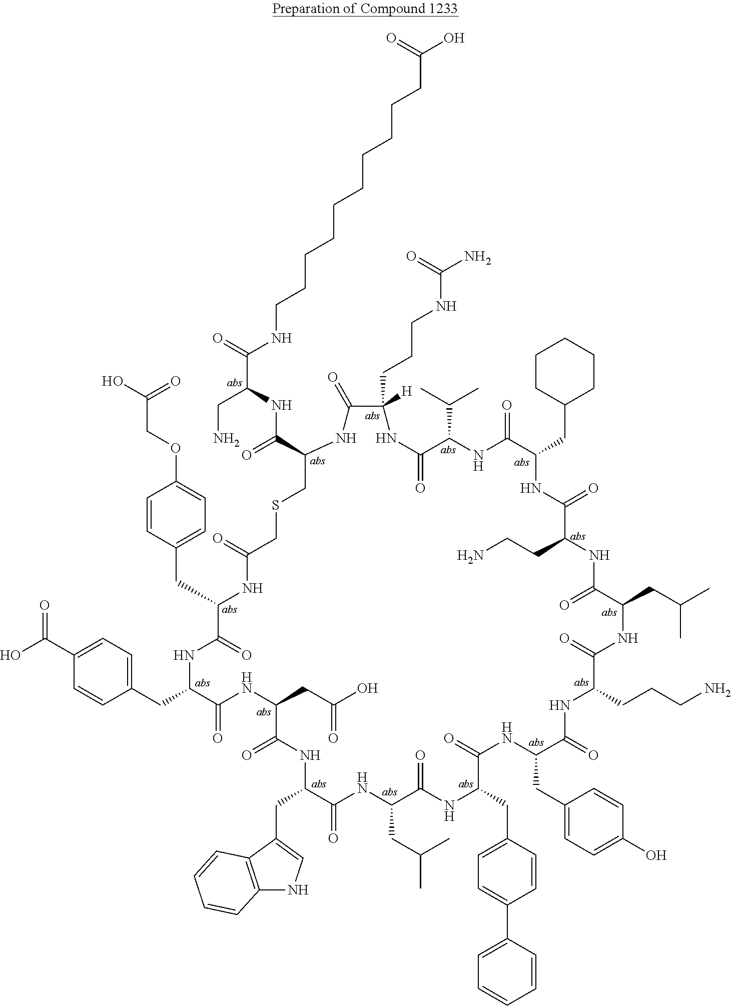 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1233 was prepared on a 50 μmol scale. The yield of the product was 15.6 mg, and its estimated purity by LCMS analysis was 100%. Analysis condition A: Retention time=1.68 min; ESI-MS(+) m/z [M+2H]²⁺: 1191.1.

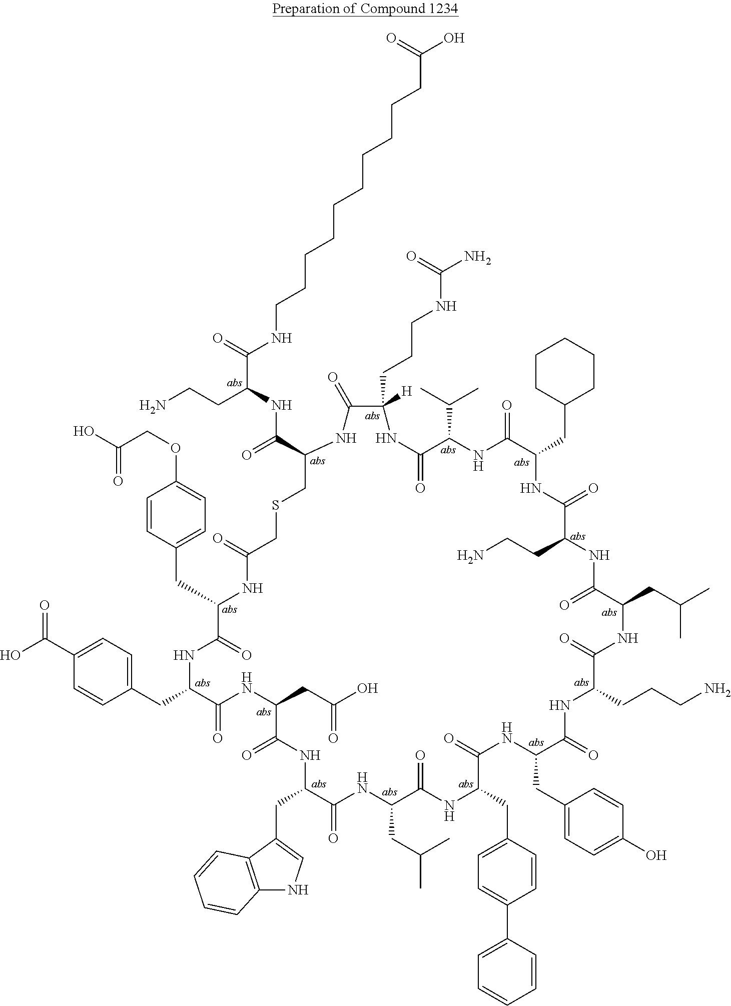 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1234 was prepared on a 50 μmol scale. The yield of the product was 18.2 mg, and its estimated purity by LCMS analysis was 86.4%. Analysis condition A: Retention time=1.56 min; ESI-MS(+) m/z [M+2H]²⁺: 1198.2.

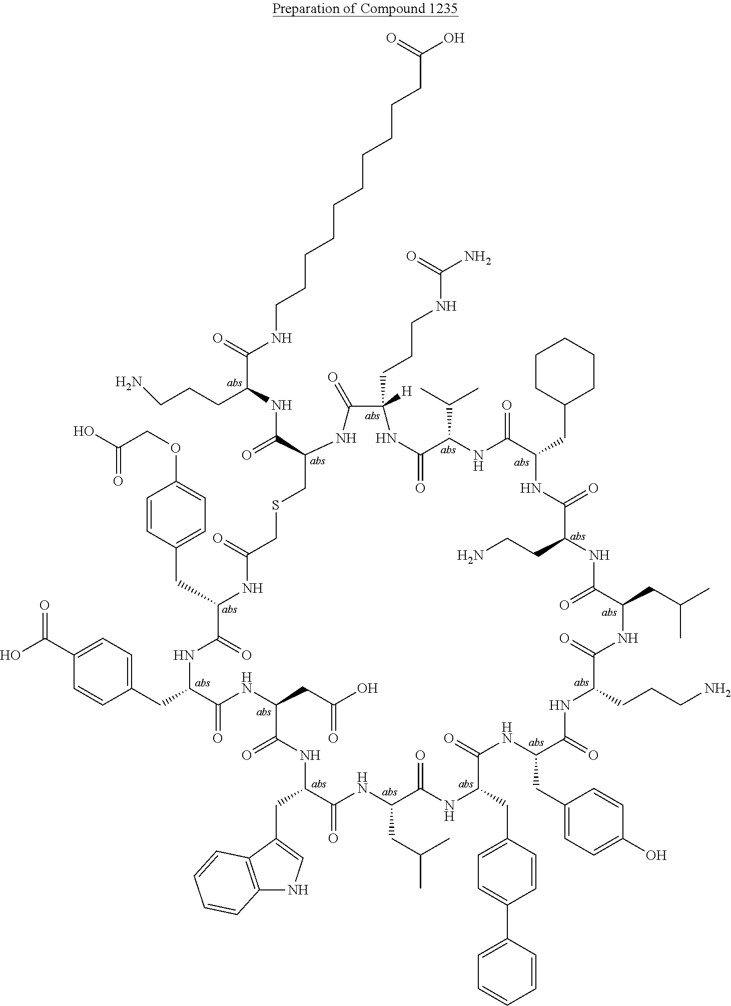 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1235 was prepared on a 50 μmol scale. The yield of the product was 22.3 mg, and its estimated purity by LCMS analysis was 85.3%. Analysis condition A: Retention time=1.52 min; ESI-MS(+) m/z [M+2H]²⁺: 1205.1.

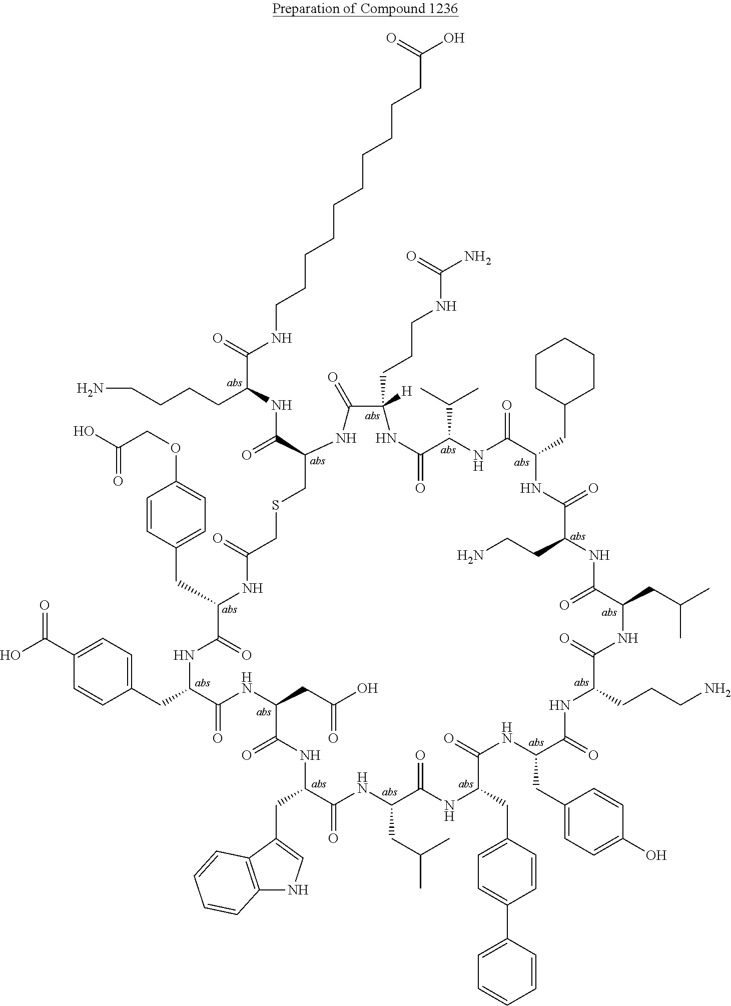 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1236 was prepared on a 50 μmol scale. The yield of the product was 12.5 mg, and its estimated purity by LCMS analysis was 87.9%. Analysis condition A: Retention time=1.52 min; ESI-MS(+) m/z [M+2H]²⁺: 1212.3.

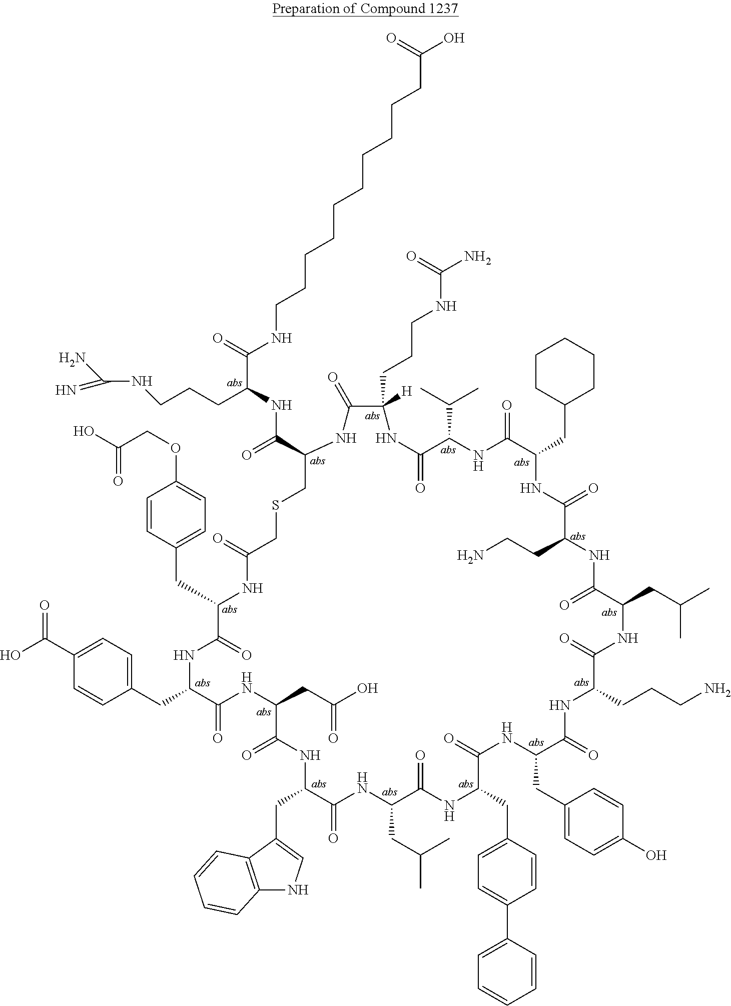 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCNC(=N)N)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1237 was prepared on a 50 μmol scale. The yield of the product was 20.4 mg, and its estimated purity by LCMS analysis was 90.2%. Analysis condition A: Retention time=1.66, 1.72 min; ESI-MS(+) m/z [M+3H]³⁺: 818.

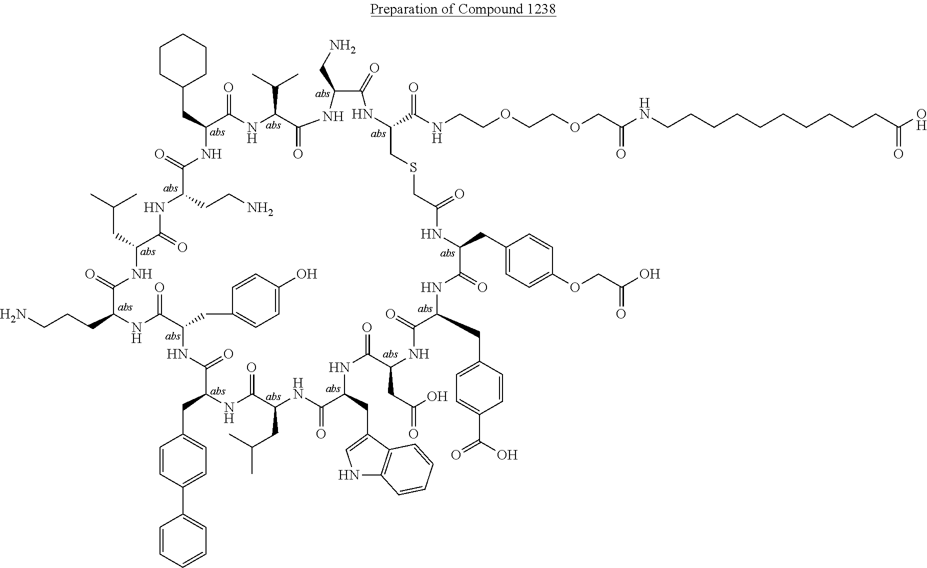 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1238 was prepared on a 50 μmol scale. The yield of the product was 55 mg, and its estimated purity by LCMS analysis was 96.3%. Analysis condition A: Retention time=1.62 min; ESI-MS(+) m/z [M+2H]²⁺: 1185.2.

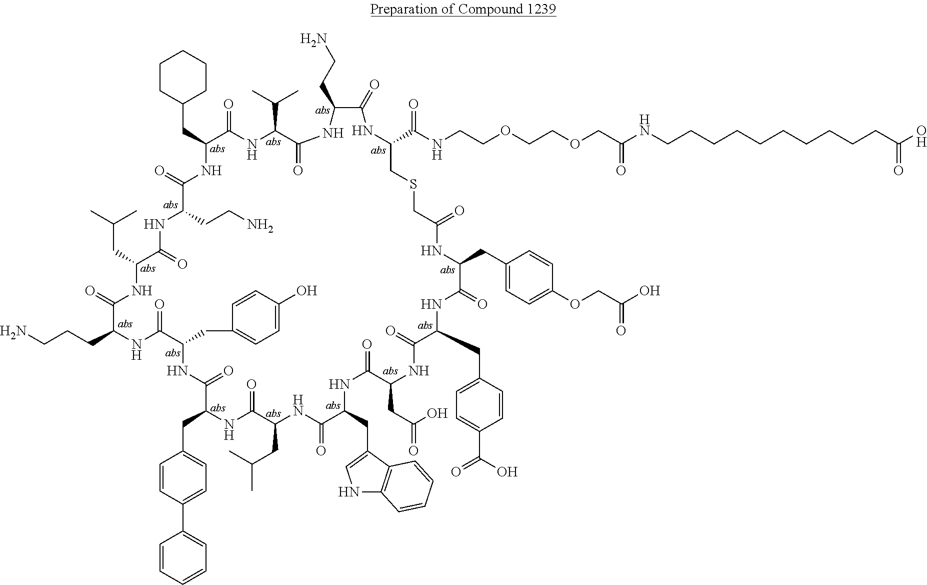 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1239 was prepared on a 50 μmol scale. The yield of the product was 9.8 mg, and its estimated purity by LCMS analysis was 98.7%. Analysis condition A: Retention time=1.59 min; ESI-MS(+) m/z [M+2H]²⁺: 1192.

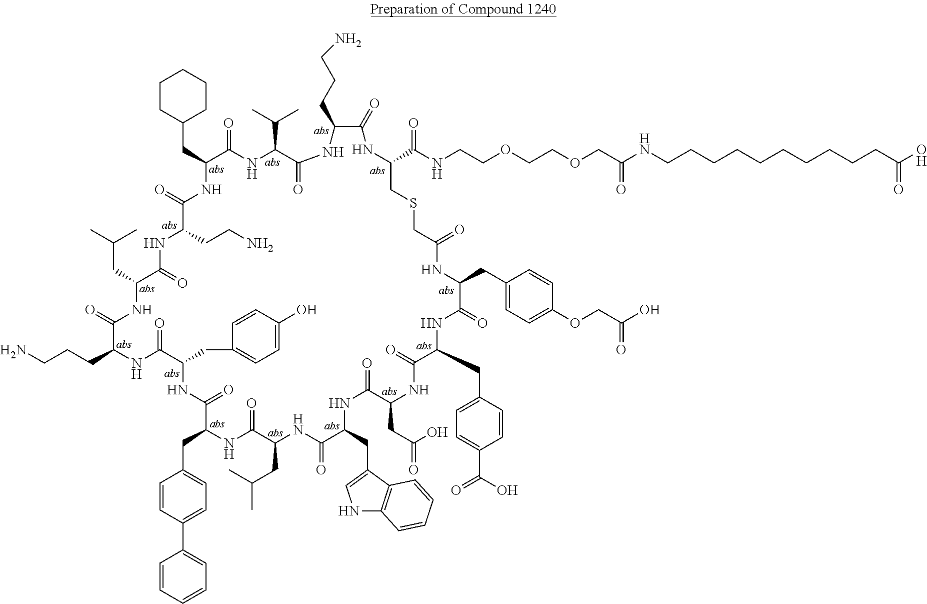 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1240 was prepared on a 50 μmol scale. The yield of the product was 6.8 mg, and its estimated purity by LCMS analysis was 94.1%. Analysis condition A: Retention time=1.66 min; ESI-MS(+) m/z [M+2H]²⁺: 1199.

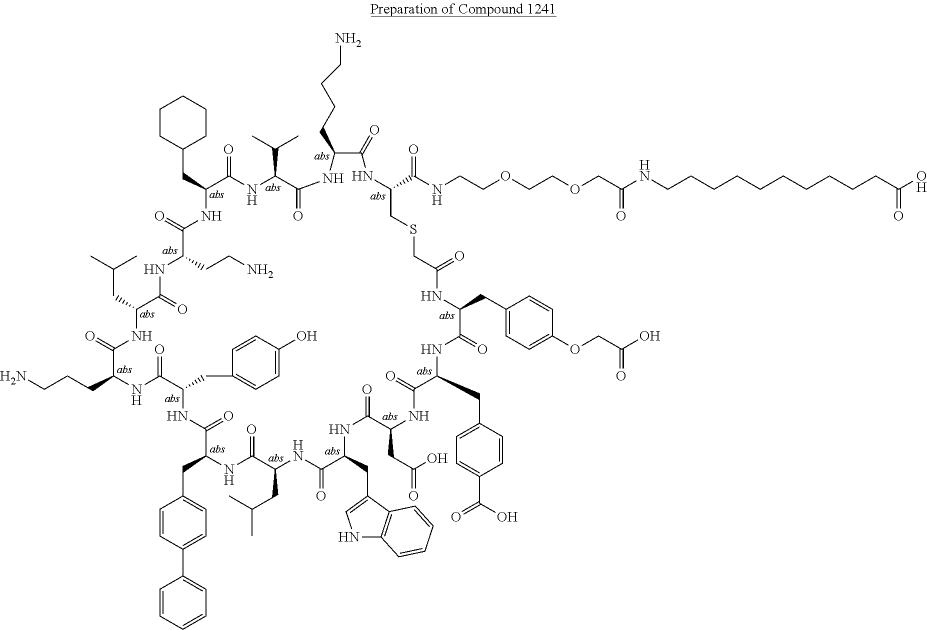 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1241 was prepared on a 50 μmol scale. The yield of the product was 10.6 mg, and its estimated purity by LCMS analysis was 96.8%. Analysis condition A: Retention time=1.66 min; ESI-MS(+) m/z [M+2H]²⁺: 1205.9.

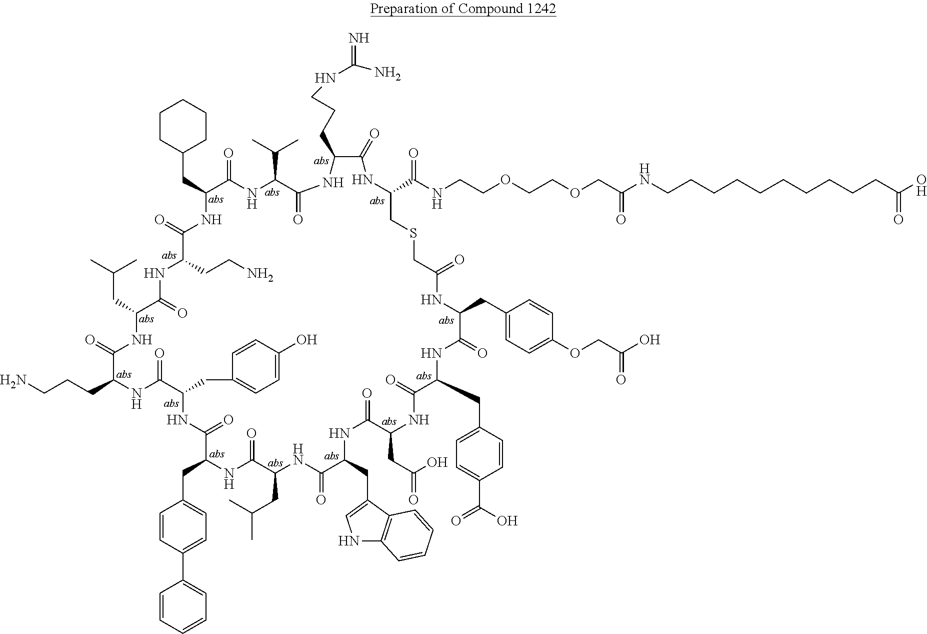 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 1242 was prepared on a 50 μmol scale. The yield of the product was 48.8 mg, and its estimated purity by LCMS analysis was 89.3%. Analysis condition A: Retention time=1.74 min; ESI-MS(+) m/z [M+2H]²⁺: 1220.2.

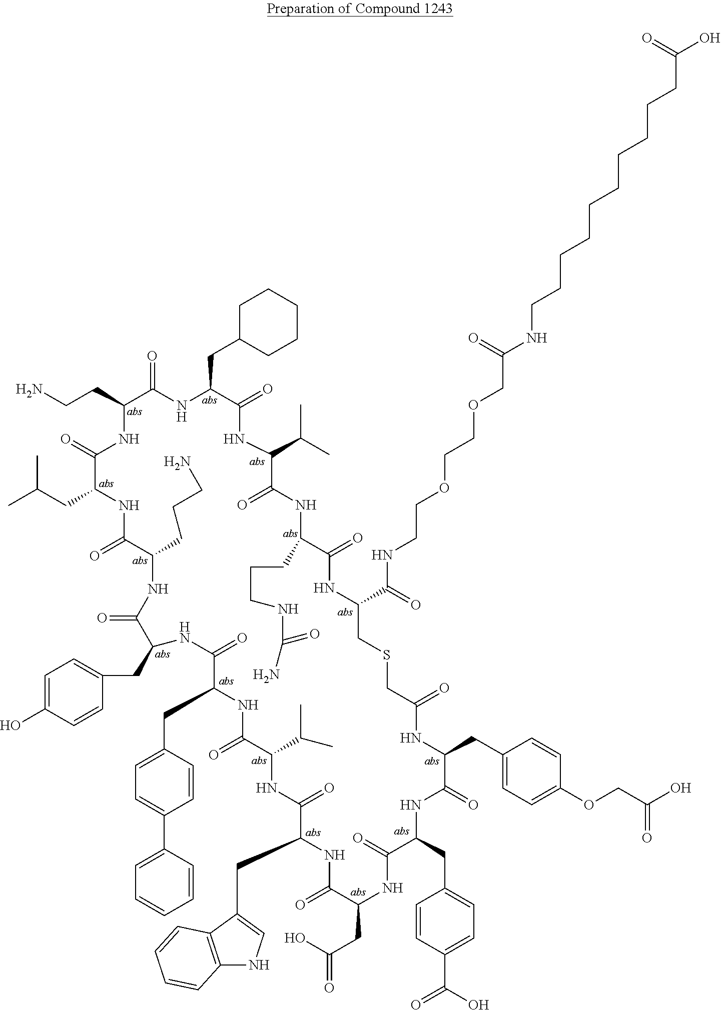

Compound 1243 was prepared on a 50 μmol scale. The yield of the product was 24 mg, and its estimated purity by LCMS analysis was 96.6%. Analysis condition B: Retention time=1.67 min; ESI-MS(+) m/z [M+2H]²⁺: 1214.0.

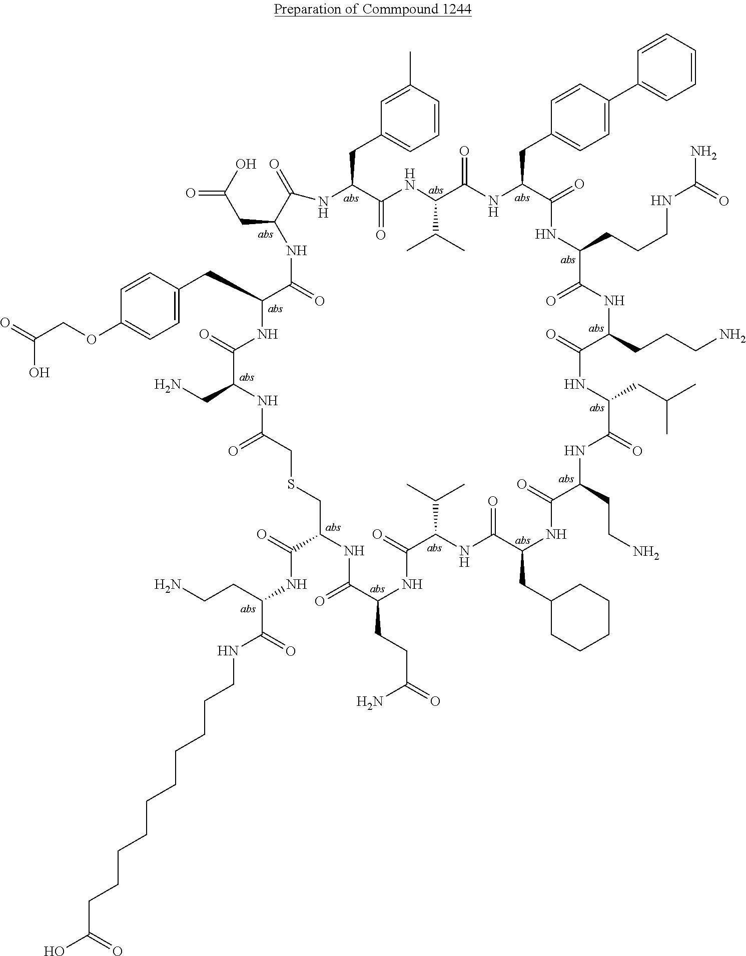 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1244 was prepared on a 50 μmol scale. The yield of the product was 1.6 mg, and its estimated purity by LCMS analysis was 83.4%. Analysis condition B: Retention time=1.6 min; ESI-MS(+) m/z [M+3H]³⁺: 739.1.

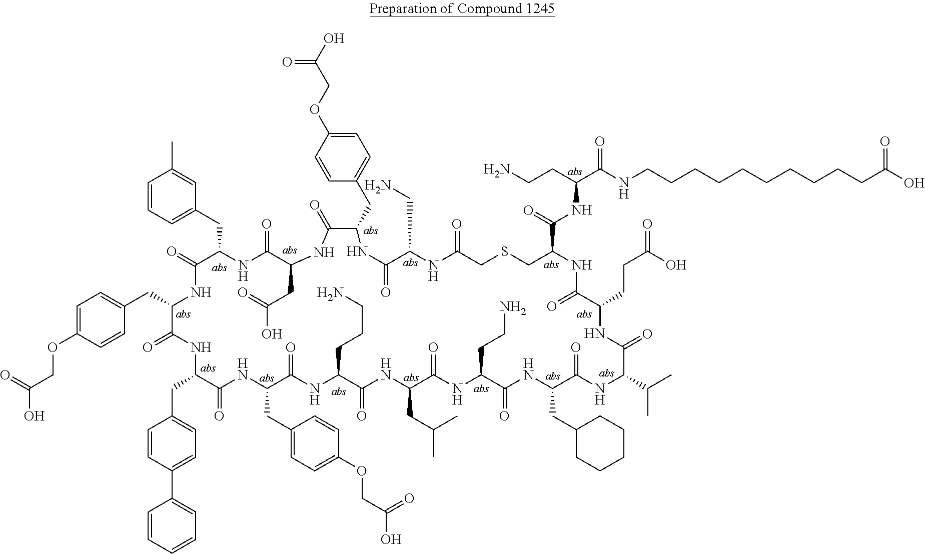 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1245 was prepared on a 50 μmol scale. The yield of the product was 1.4 mg, and its estimated purity by LCMS analysis was 71.7%. Analysis condition B: Retention time=1.64 min; ESI-MS(+) m/z [M+3H]³⁺: 801.1.

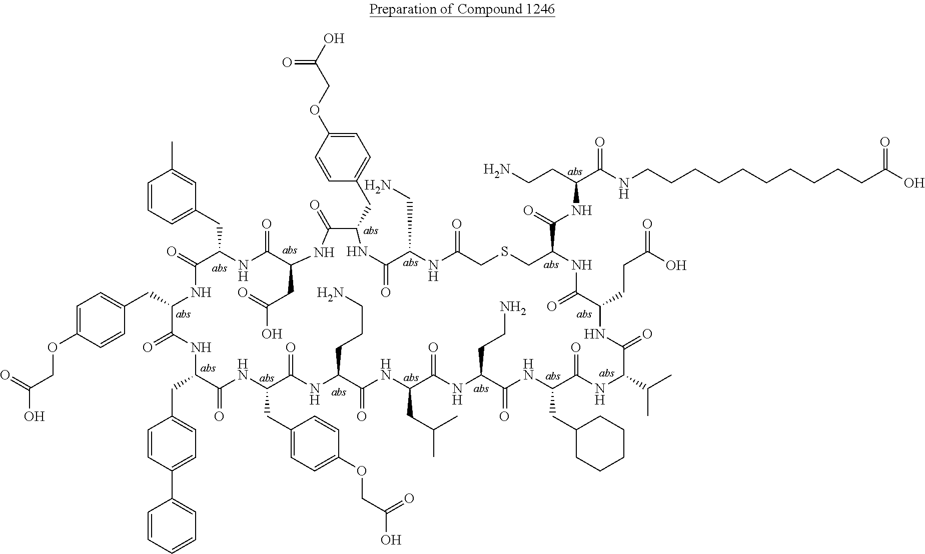 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1246 was prepared on a 50 μmol scale. The yield of the product was 2.4 mg, and its estimated purity by LCMS analysis was 89.1%. Analysis condition B: Retention time=1.64 min; ESI-MS(+) m/z [M+3H]³⁺: 797.1.

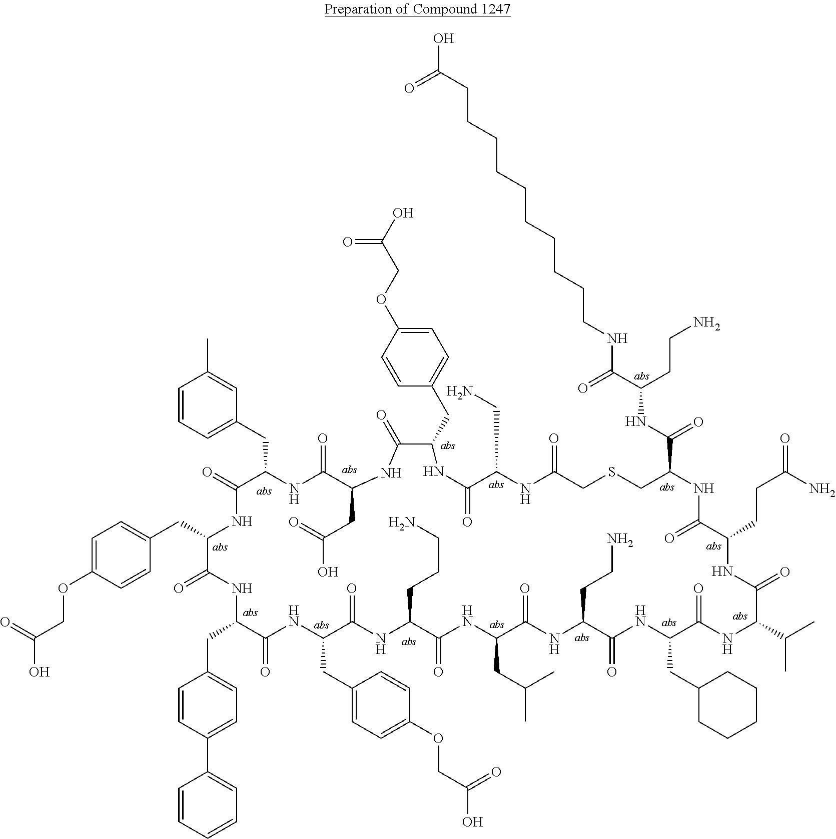 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1247 was prepared on a 50 μmol scale. The yield of the product was 4 mg, and its estimated purity by LCMS analysis was 80.3%. Analysis condition B: Retention time=1.62 min; ESI-MS(+) m/z [M+3H]³⁺: 801.1.

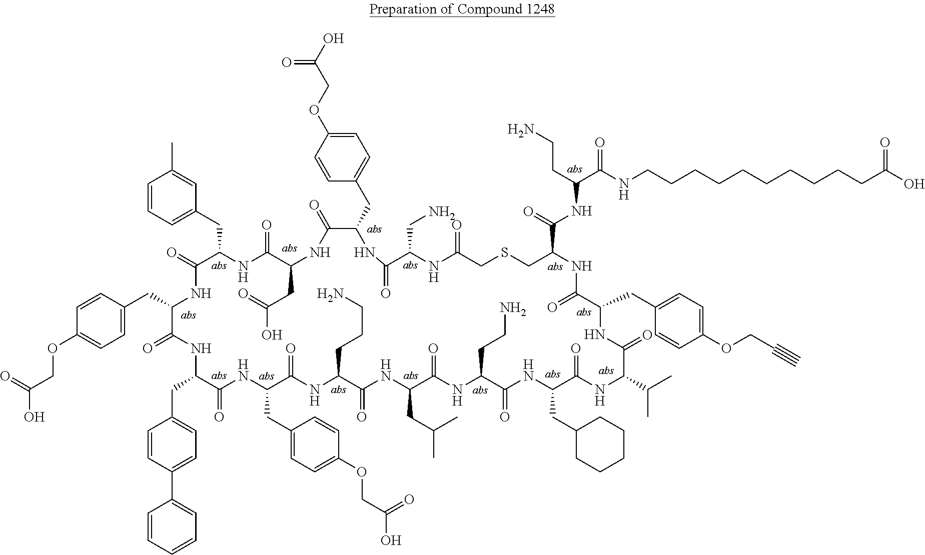

Compound 1248 was prepared on a 50 μmol scale. The yield of the product was 2.1 mg, and its estimated purity by LCMS analysis was 100%. Analysis condition A: Retention time=1.63 min; ESI-MS(+) m/z [M+3H]³⁺: 825.9.

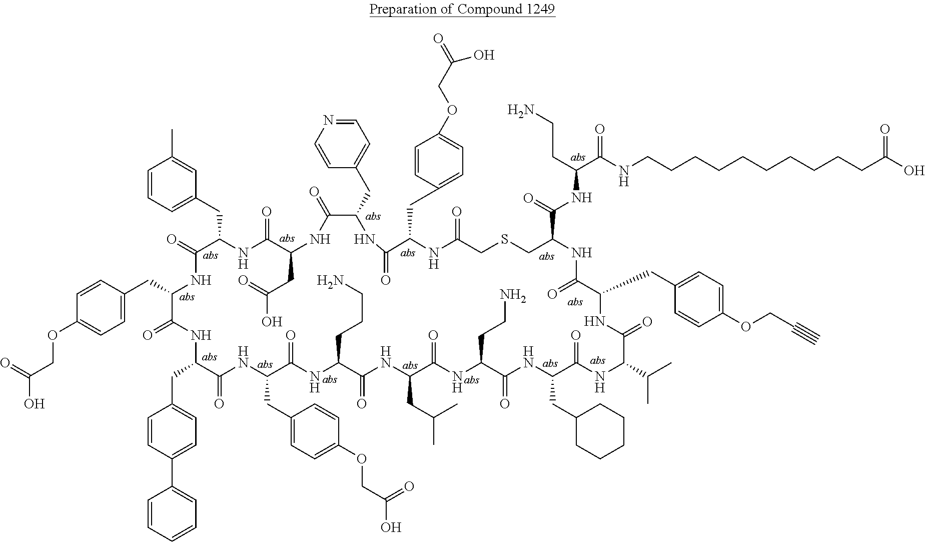 [SMILES: CCCOc1ccc(C[C@@H]2NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC2=O)cc1]

Compound 1249 was prepared on a 50 μmol scale. The yield of the product was 24.1 mg, and its estimated purity by LCMS analysis was 86%. Analysis condition A: Retention time=1.55 min; ESI-MS(+) m/z [M+2H]²⁺: 1268.9.

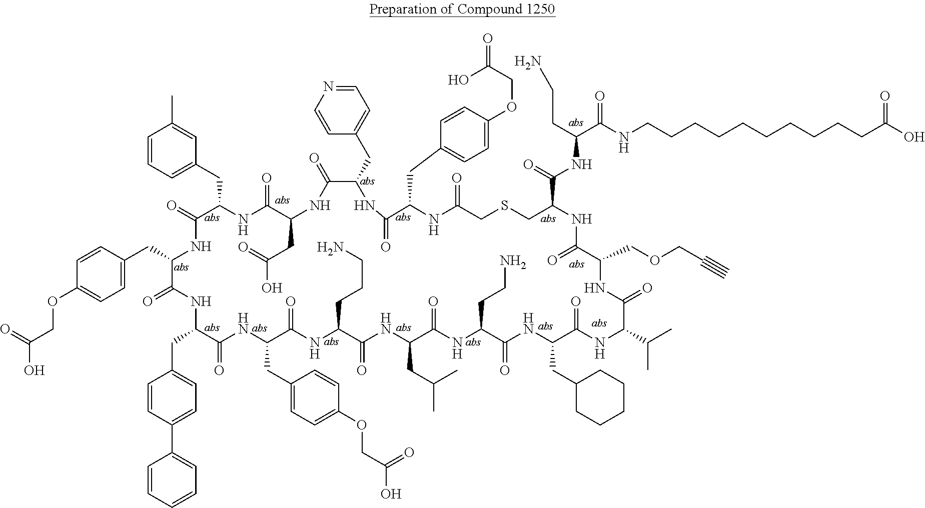 [SMILES: CCCOC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC1=O]

Compound 1250 was prepared on a 50 μmol scale. The yield of the product was 0.8 mg, and its estimated purity by LCMS analysis was 85.8%. Analysis condition B: Retention time=1.61 min; ESI-MS(+) m/z [M+2H]²⁺: 1231.3.

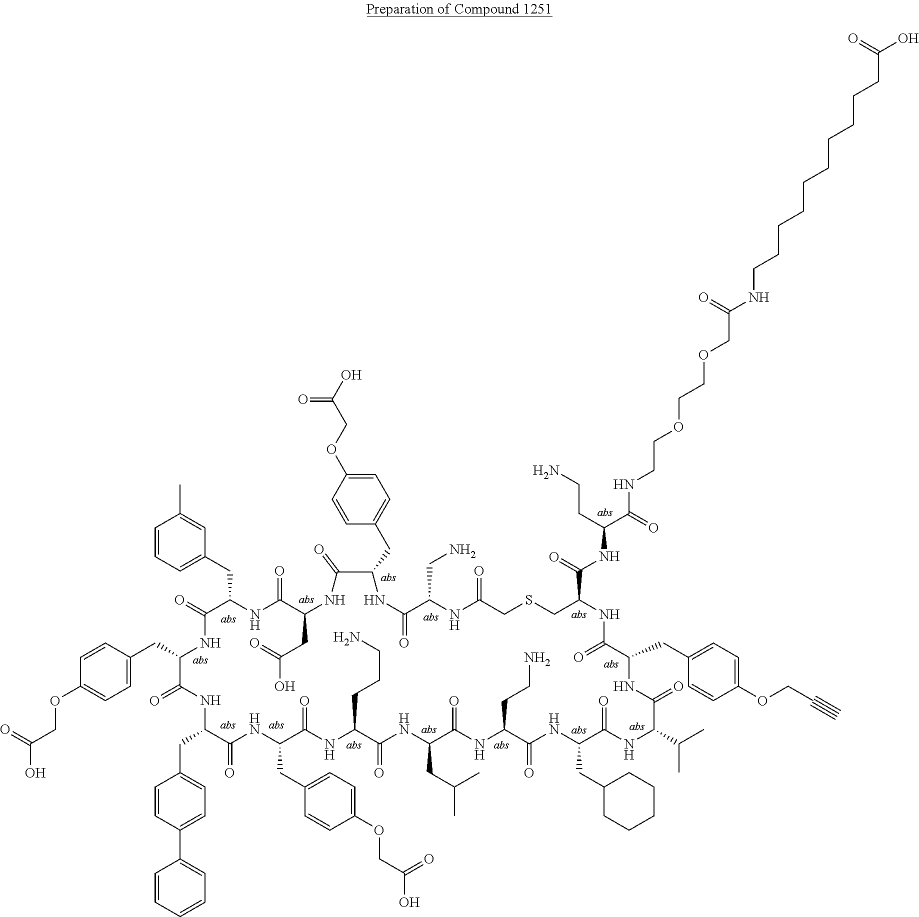 [SMILES: CCCOc1ccc(C[C@@H]2NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC2=O)cc1]

Compound 1251 was prepared on a 50 μmol scale. The yield of the product was 21.2 mg, and its estimated purity by LCMS analysis was 85.4%. Analysis condition A: Retention time=1.63 min; ESI-MS(+) m/z [M+2H]²⁺: 1310.9.

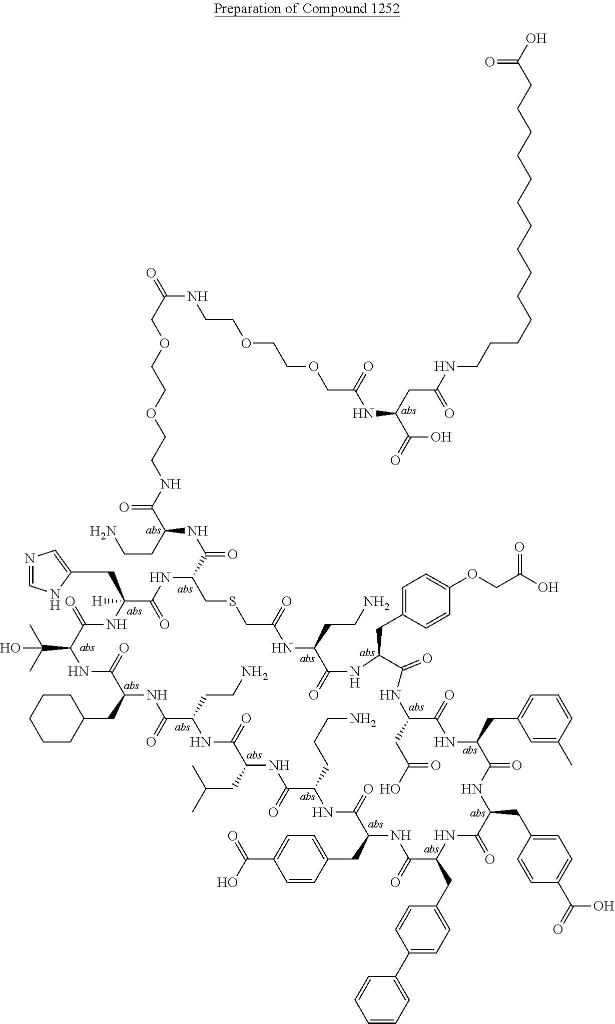 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)N[C@@H](CC(=O)NCCCCCCCCCCCCCCC(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 1252 was prepared on a 50 μmol scale. The yield of the product was 5.9 mg, and its estimated purity by LCMS analysis was 100%. Analysis condition A: Retention time=1.54 min; ESI-MS(+) m/z [M+3H]³⁺: 948.1.

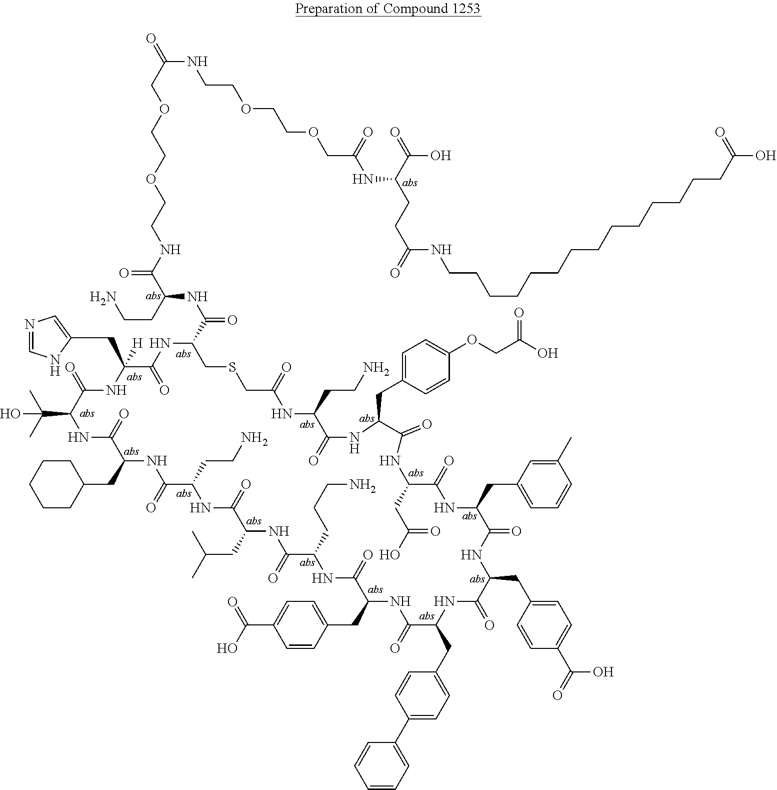 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)N[C@@H](CCC(=O)NCCCCCCCCCCCCCCC(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 1253 was prepared on a 50 μmol scale. The yield of the product was 5.4 mg, and its estimated purity by LCMS analysis was 100%. Analysis condition A: Retention time=1.61 min; ESI-MS(+) m/z [M+2H]²⁺: 1429.

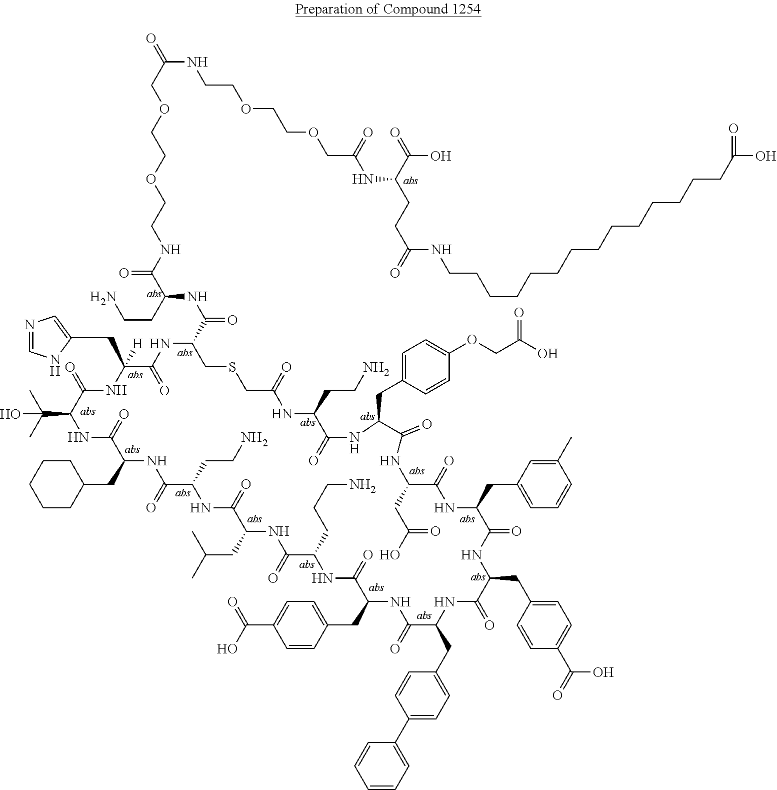 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)N[C@@H](CCC(=O)NCCCCCCCCCCCCCCC(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 1254 was prepared on a 50 μmol scale. The yield of the product was 4.3 mg, and its estimated purity by LCMS analysis was 94.8%. Analysis condition B: Retention time=1.59 min; ESI-MS(+) m/z [M+2H]²⁺: 943.2.

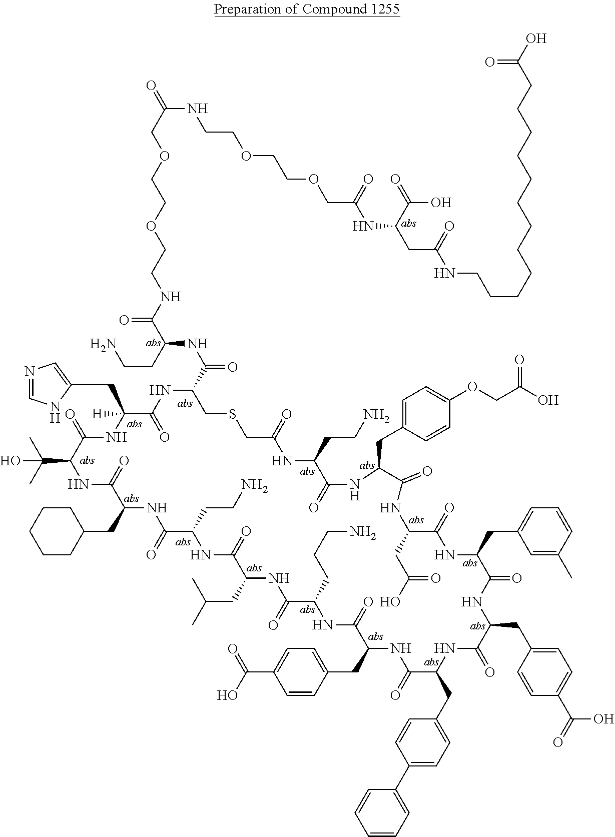 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)N[C@@H](CC(=O)NCCCCCCCCCCCCC(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 1255 was prepared on a 50 μmol scale. The yield of the product was 6.3 mg, and its estimated purity by LCMS analysis was 99.2%. Analysis condition B: Retention time=1.57 min; ESI-MS(+) m/z [M+2H]²⁺: 1407.1.

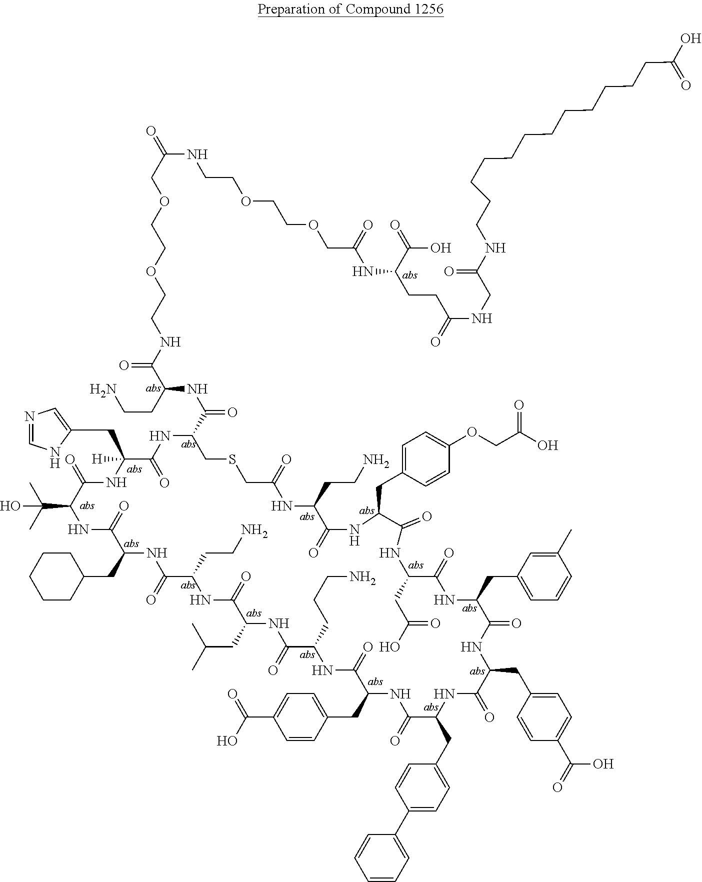 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)N[C@@H](CCC(=O)NCC(=O)NCCCCCCCCCCCCC(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 1256 was prepared on a 50 μmol scale. The yield of the product was 9.4 mg, and its estimated purity by LCMS analysis was 96.3%. Analysis condition B: Retention time=1.56 min; ESI-MS(+) m/z [M+2H]²⁺: 1443.1.

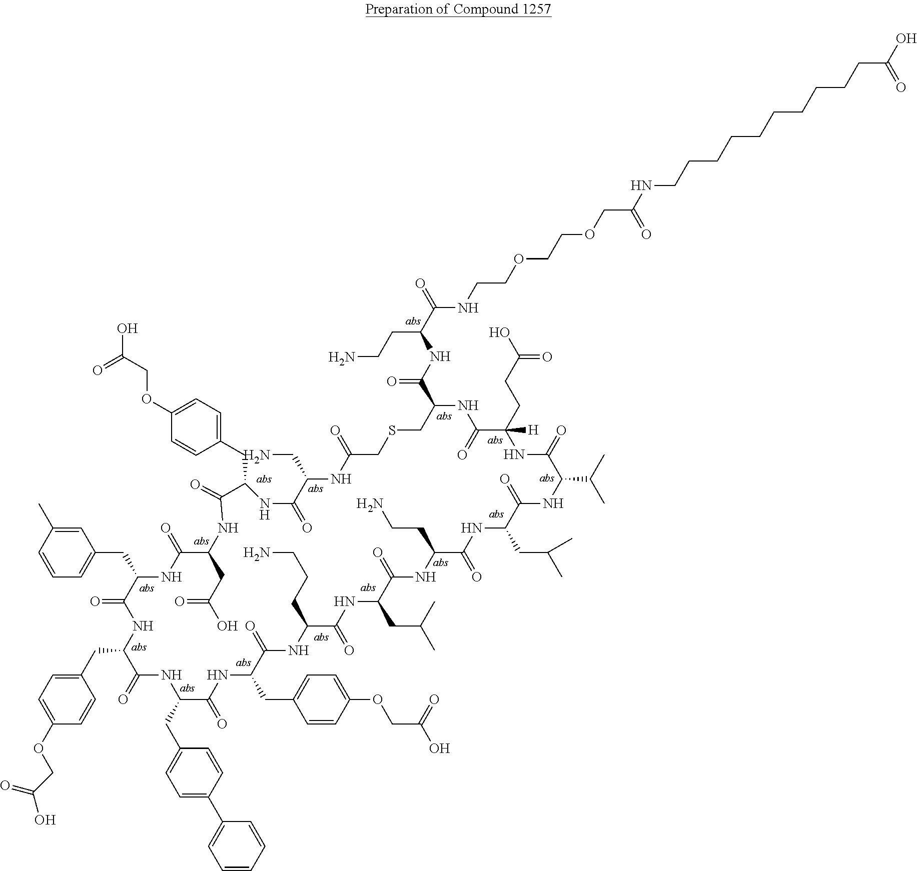 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1257 was prepared on a 50 μmol scale. The yield of the product was 3.8 mg, and its estimated purity by LCMS analysis was 100%. Analysis condition A: Retention time=1.38 min; ESI-MS(+) m/z [M+2H]²⁺: 1254.3.

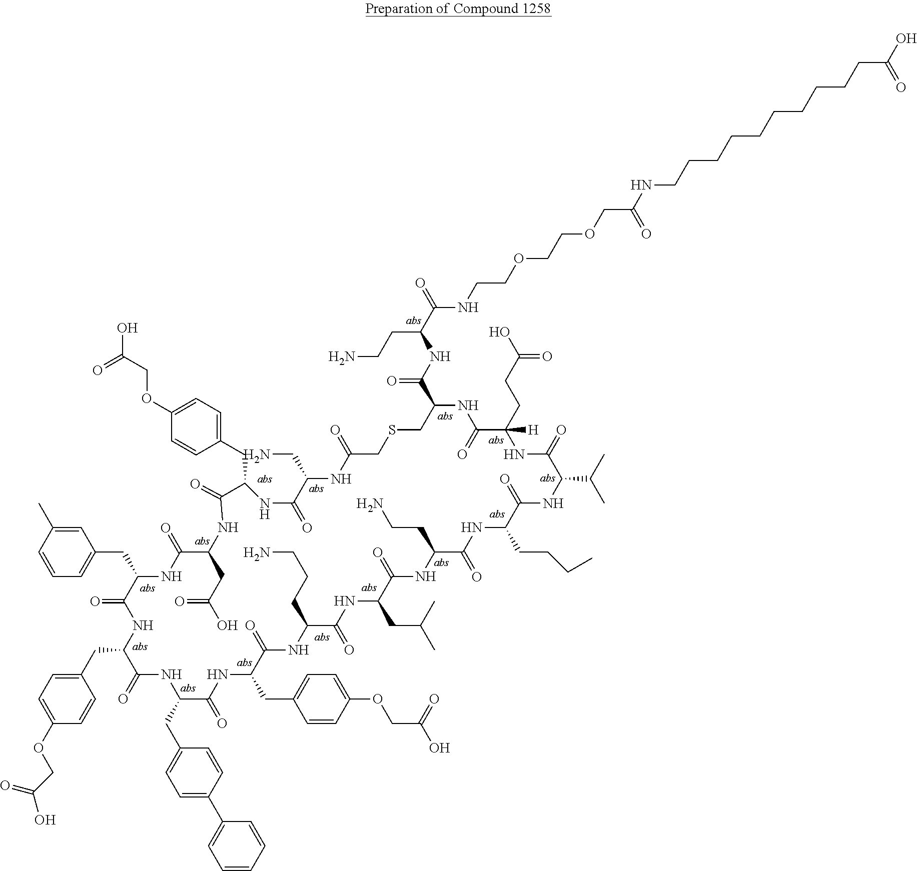 [SMILES: CCCC[C@@H]1NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC1=O]

Compound 1258 was prepared on a 50 μmol scale. The yield of the product was 5.2 mg, and its estimated purity by LCMS analysis was 97.5%. Analysis condition A: Retention time=1.42 min; ESI-MS(+) m/z [M+2H]²⁺: 1255.1.

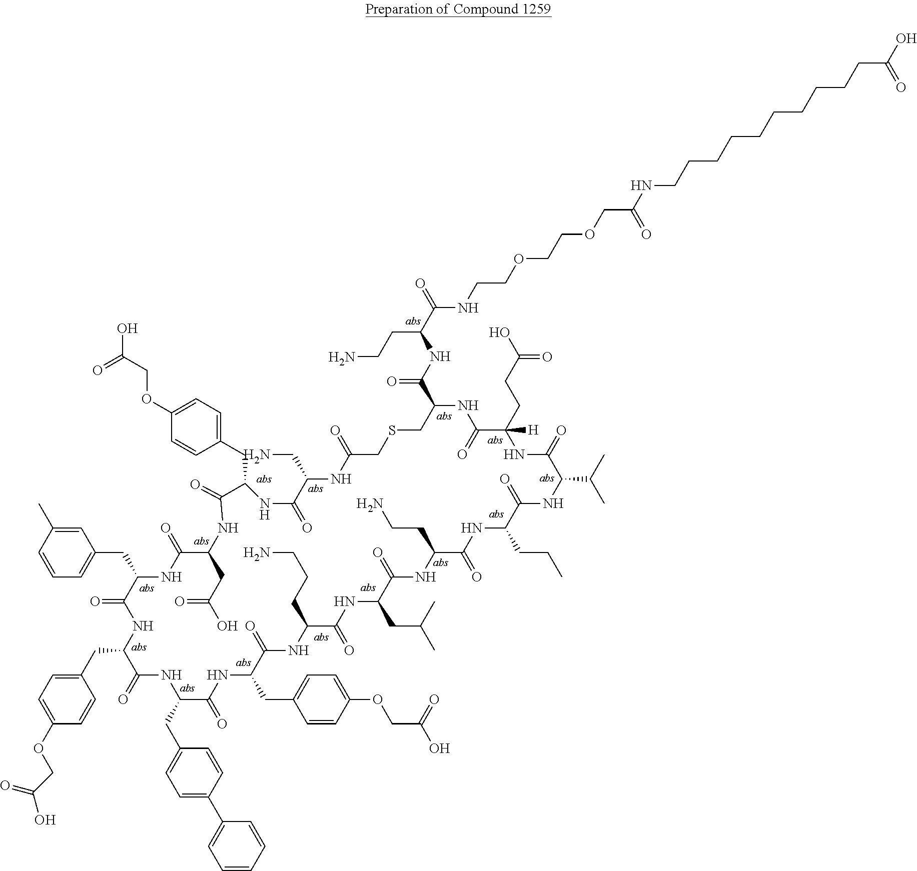 [SMILES: CCC[C@@H]1NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC1=O]

Compound 1259 was prepared on a 50 μmol scale. The yield of the product was 3.4 mg, and its estimated purity by LCMS analysis was 100%. Analysis condition A: Retention time=1.35 min; ESI-MS(+) m/z [M+3H]³⁺: 831.5.

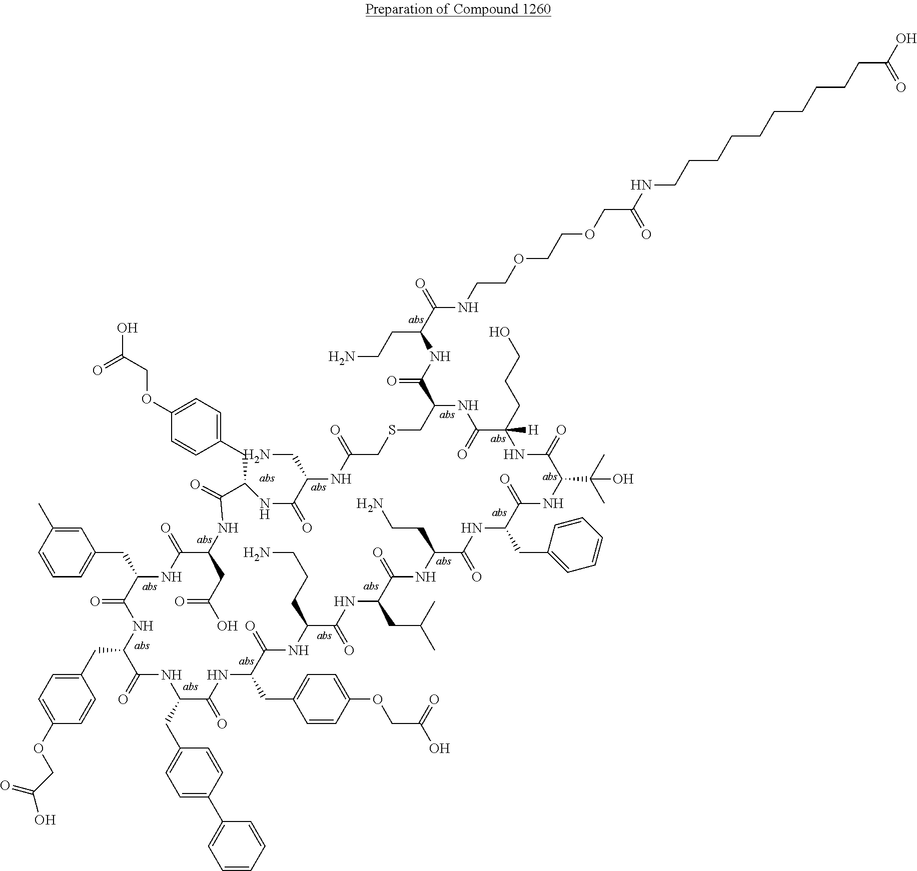 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCO)NC(=O)[C@H](C(C)(C)O)NC(=O)[C@H](Cc3ccccc3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1260 was prepared on a 50 μmol scale. The yield of the product was 8.1 mg, and its estimated purity by LCMS analysis was 100%. Analysis condition A: Retention time=1.47 min; ESI-MS(+) m/z [M+3H]³⁺: 839.2.

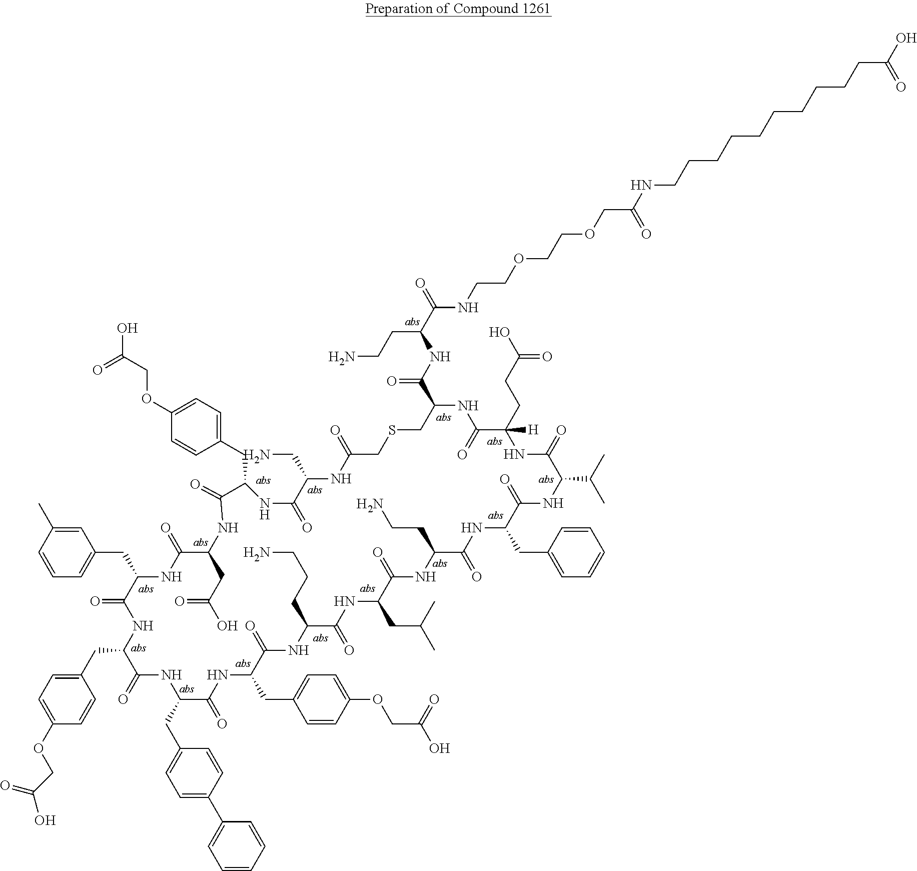 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3ccccc3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1261 was prepared on a 50 μmol scale. The yield of the product was 7.6 mg, and its estimated purity by LCMS analysis was 100%. Analysis condition B: Retention time=1.52 min; ESI-MS(+) m/z [M+3H]³⁺: 848.

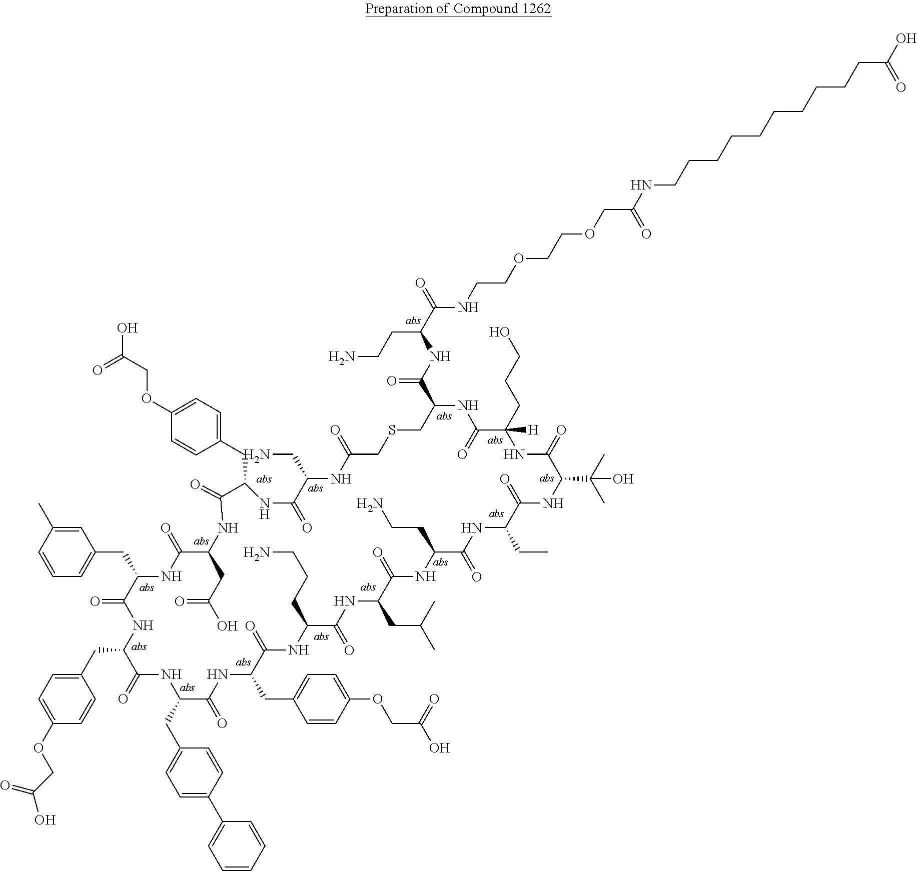 [SMILES: CC[C@@H]1NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCO)NC(=O)[C@H](C(C)(C)O)NC1=O]

Compound 1262 was prepared on a 50 μmol scale. The yield of the product was 9.4 mg, and its estimated purity by LCMS analysis was 96.4%. Analysis condition B: Retention time=1.46 min; ESI-MS(+) m/z [M+3H]³⁺: 1227.3.

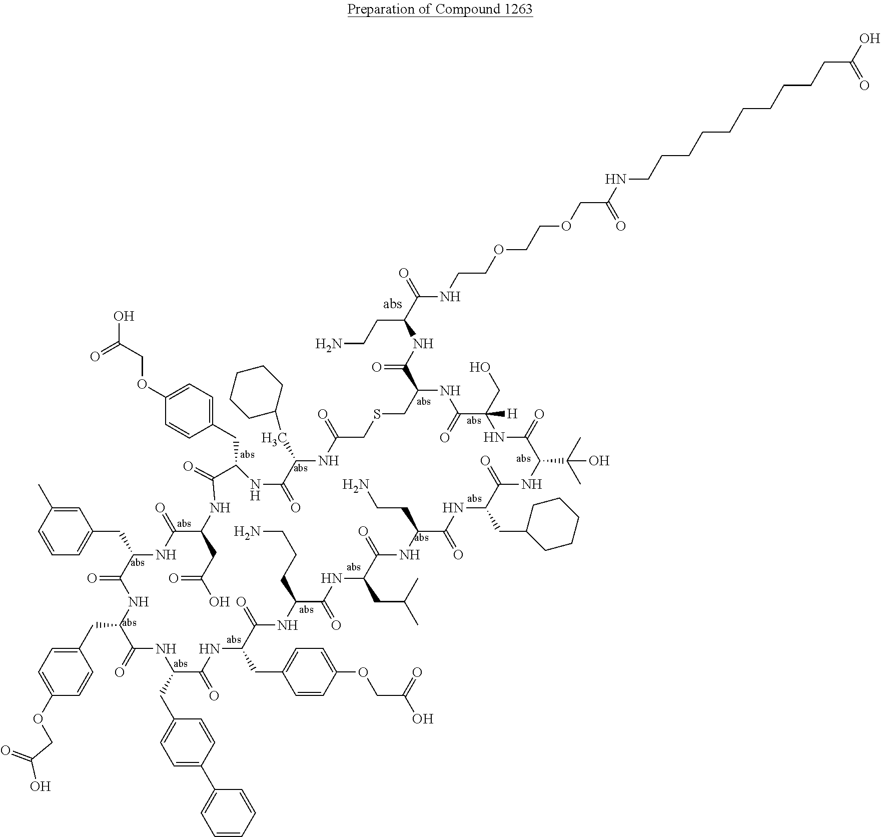 [SMILES: Cc1cccc(C[C@@H]2NC(=O)[C@H](CC(=O)O)NC(=O)[C@H](Cc3ccc(OCC(=O)O)cc3)NC(=O)[C@H](CC3CCCCC3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1263 was prepared on a 50 μmol scale. The yield of the product was 10.7 mg, and its estimated purity by LCMS analysis was 100%. Analysis condition A: Retention time=1.57 min; ESI-MS(+) m/z [M+2H]²⁺: 1295.1.

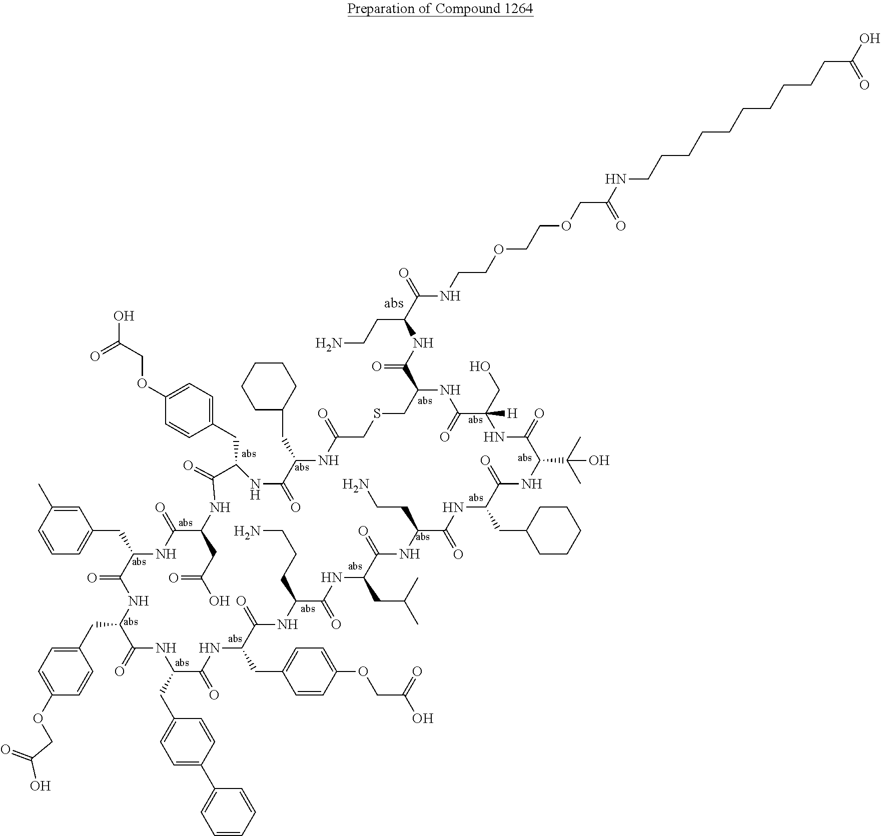 [SMILES: Cc1cccc(C[C@@H]2NC(=O)[C@H](CC(=O)O)NC(=O)[C@H](Cc3ccc(OCC(=O)O)cc3)NC(=O)[C@H](CC3CCCCC3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1264 was prepared on a 50 μmol scale. The yield of the product was 25.9 mg, and its estimated purity by LCMS analysis was 97.9%. Analysis condition A: Retention time=1.49 min; ESI-MS(+) m/z [M+2H]²⁺: 1316.1.

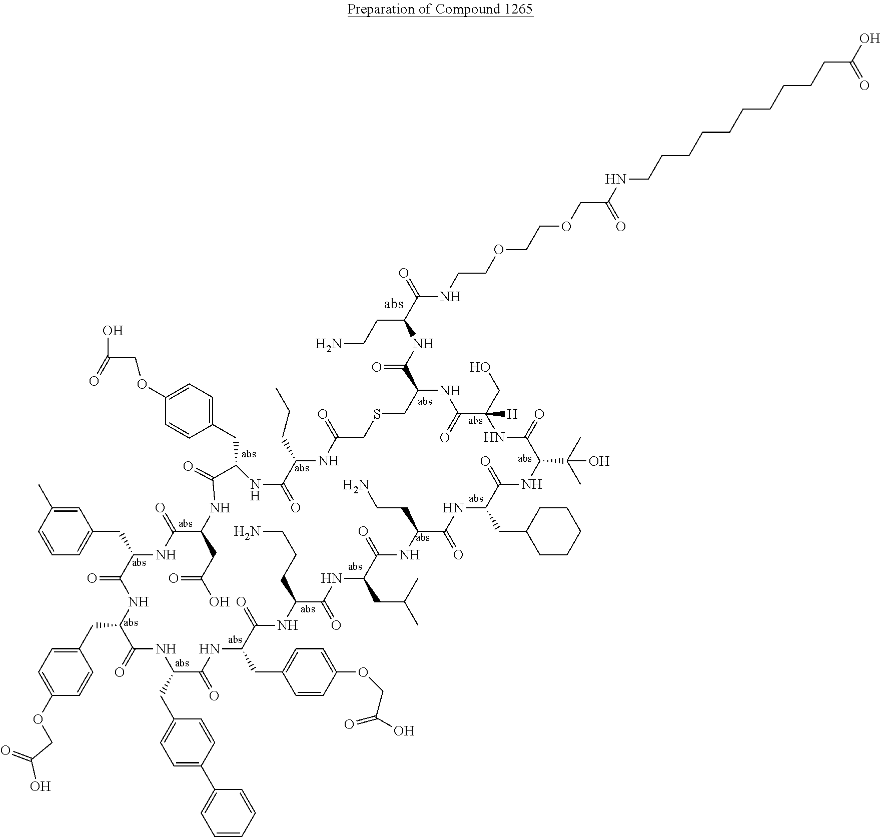 [SMILES: CCC[C@@H]1NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 1265 was prepared on a 50 μmol scale. The yield of the product was 28.4 mg, and its estimated purity by LCMS analysis was 97.9%. Analysis condition A: Retention time=1.4 min; ESI-MS(+) m/z [M+2H]²⁺: 1289.2.

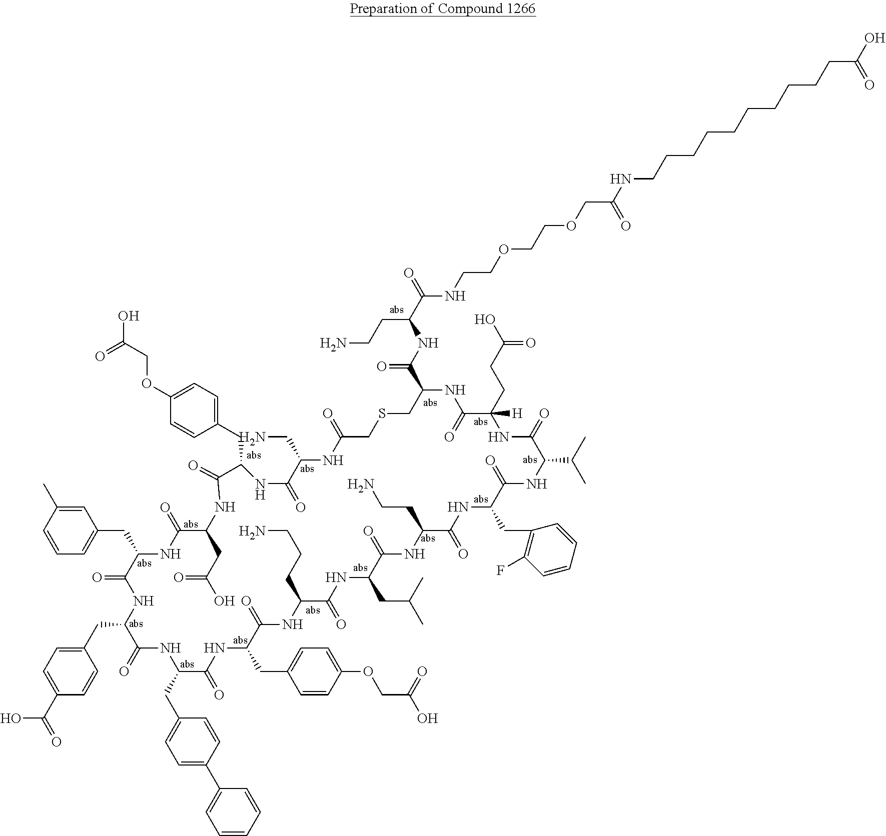 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3ccccc3F)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1266 was prepared on a 50 μmol scale. The yield of the product was 4.1 mg, and its estimated purity by LCMS analysis was 100%. Analysis condition A: Retention time=1.37 min; ESI-MS(+) m/z [M+3H]³⁺: 844.

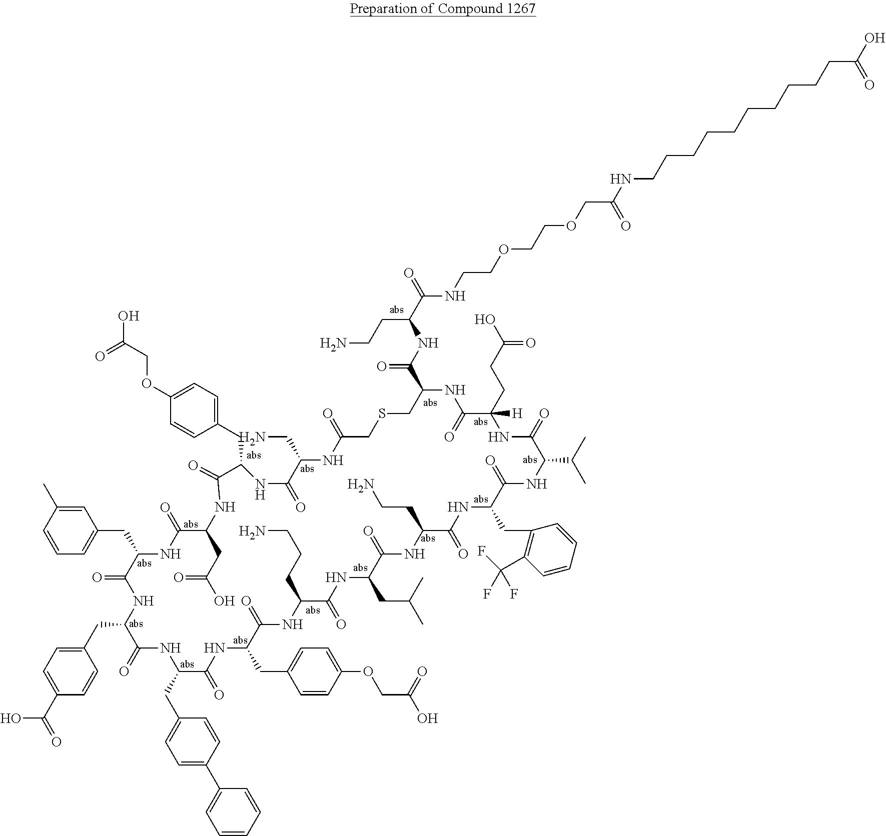 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3ccccc3C(F)(F)F)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1267 was prepared on a 50 μmol scale. The yield of the product was 14.5 mg, and its estimated purity by LCMS analysis was 100%. Analysis condition A: Retention time=1.41 min; ESI-MS(+) m/z [M+3H]³⁺: 860.4.

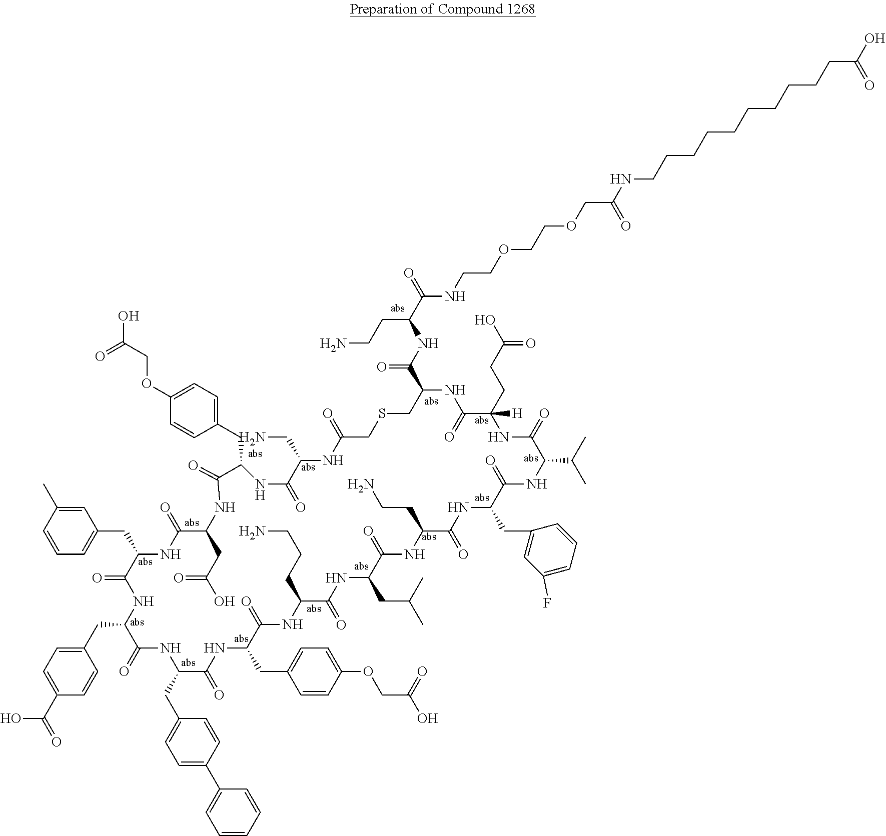 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3cccc(F)c3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1268 was prepared on a 50 μmol scale. The yield of the product was 12 mg, and its estimated purity by LCMS analysis was 96.5%. Analysis condition A: Retention time=1.34 min; ESI-MS(+) m/z [M+2H]²⁺: 1265.9.

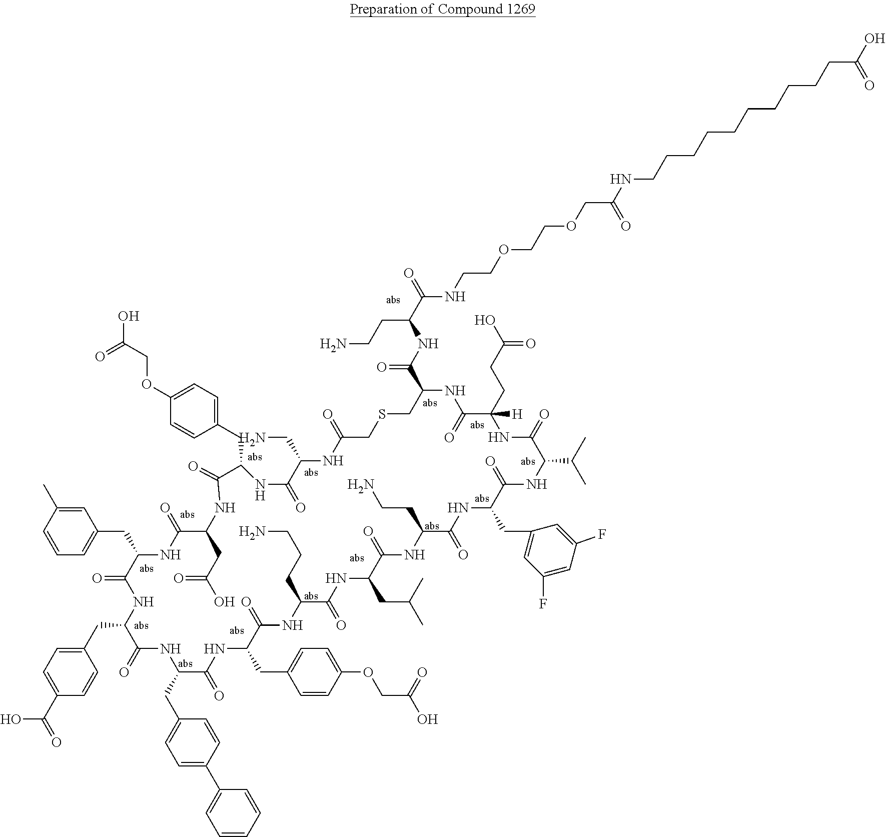 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3cc(F)cc(F)c3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1269 was prepared on a 50 μmol scale. The yield of the product was 6.5 mg, and its estimated purity by LCMS analysis was 98.8%. Analysis condition A: Retention time=1.44 min; ESI-MS(+) m/z [M+2H]²⁺: 1274.3.

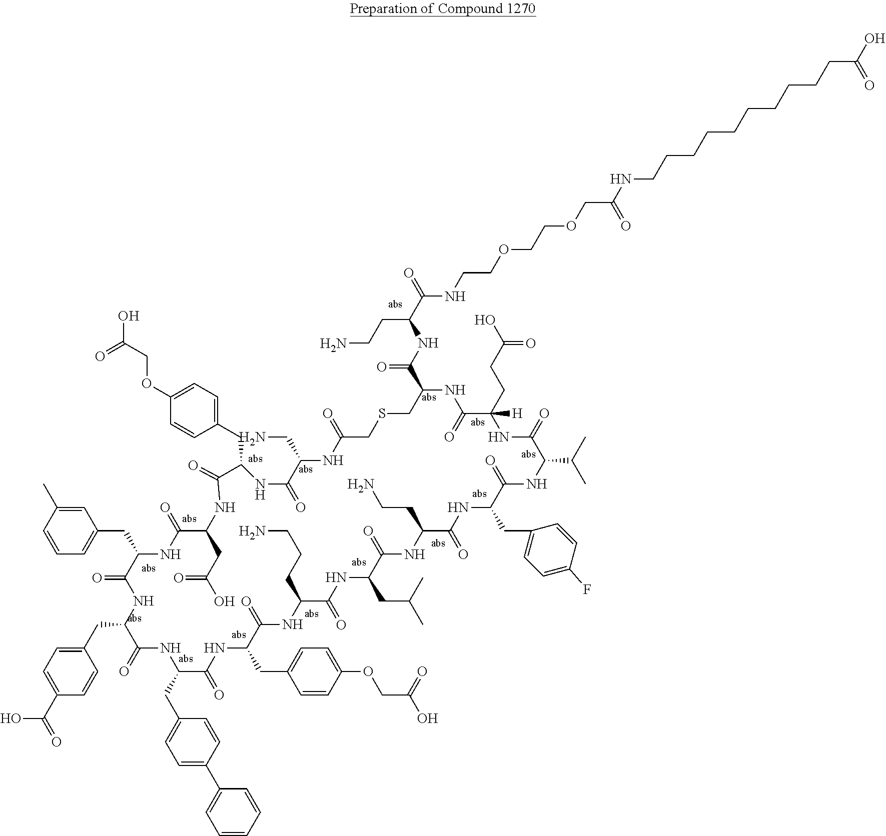 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3ccc(F)cc3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1270 was prepared on a 50 μmol scale. The yield of the product was 4.3 mg, and its estimated purity by LCMS analysis was 100%. Analysis condition A: Retention time=1.35 min; ESI-MS(+) m/z [M+3H]³⁺: 844.

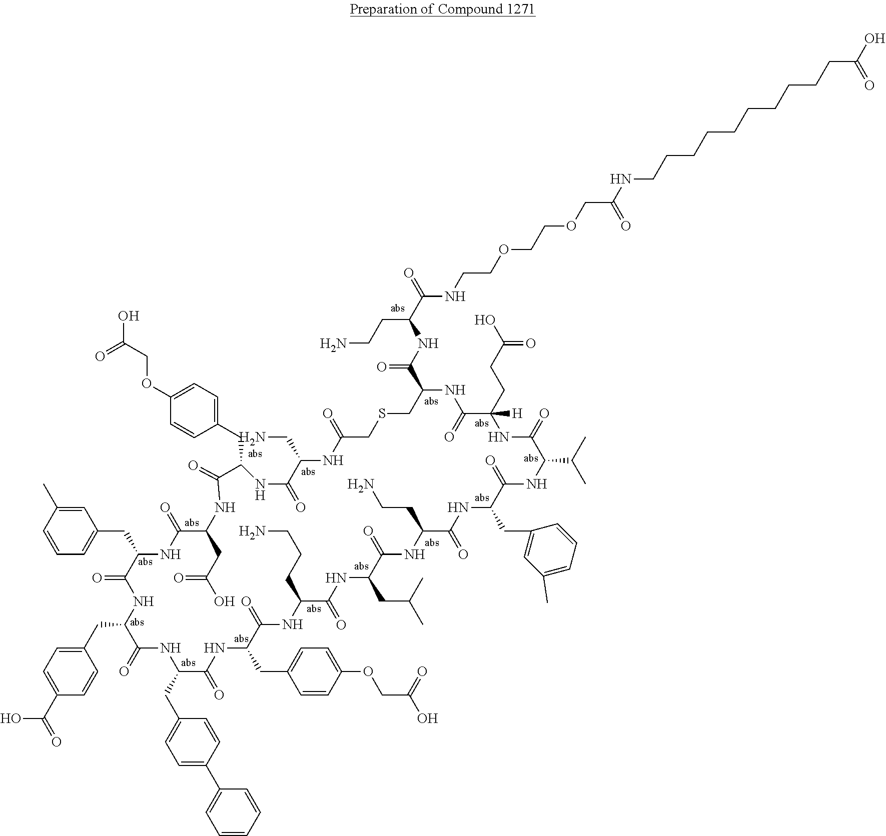 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3cccc(C)c3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1271 was prepared on a 50 μmol scale. The yield of the product was 5.9 mg, and its estimated purity by LCMS analysis was 100%. Analysis condition B: Retention time=1.54 min; ESI-MS(+) m/z [M+3H]³⁺: 842.

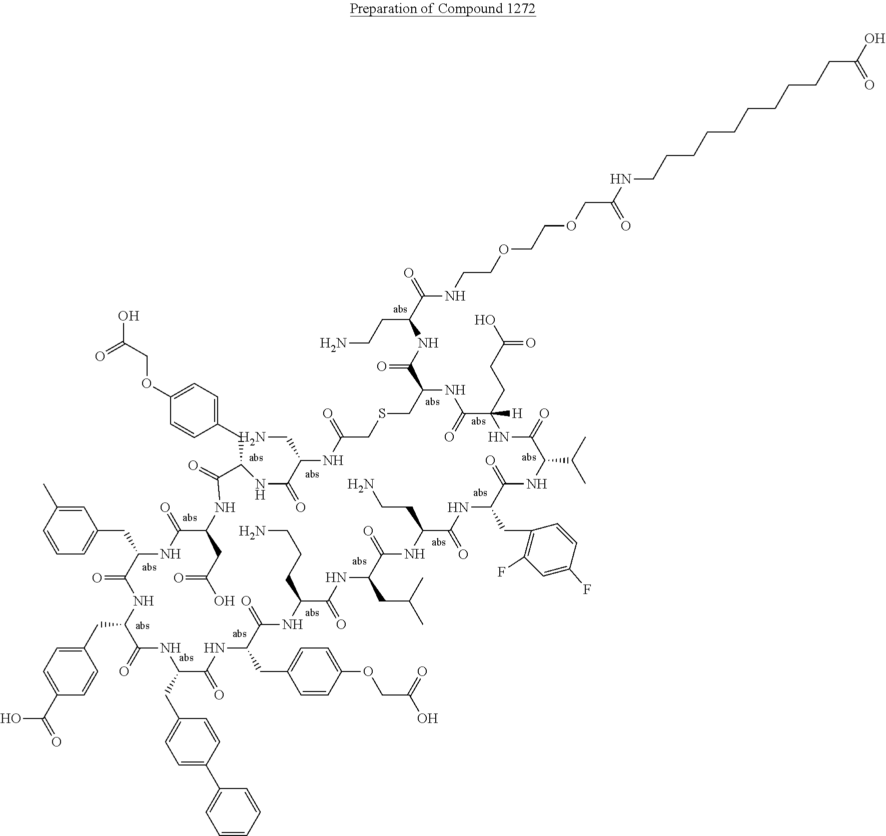 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3ccc(F)cc3F)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1272 was prepared on a 50 μmol scale. The yield of the product was 16.3 mg, and its estimated purity by LCMS analysis was 95%. Analysis condition B: Retention time=1.52 min; ESI-MS(+) m/z [M+2H]²⁺: 1274.3.

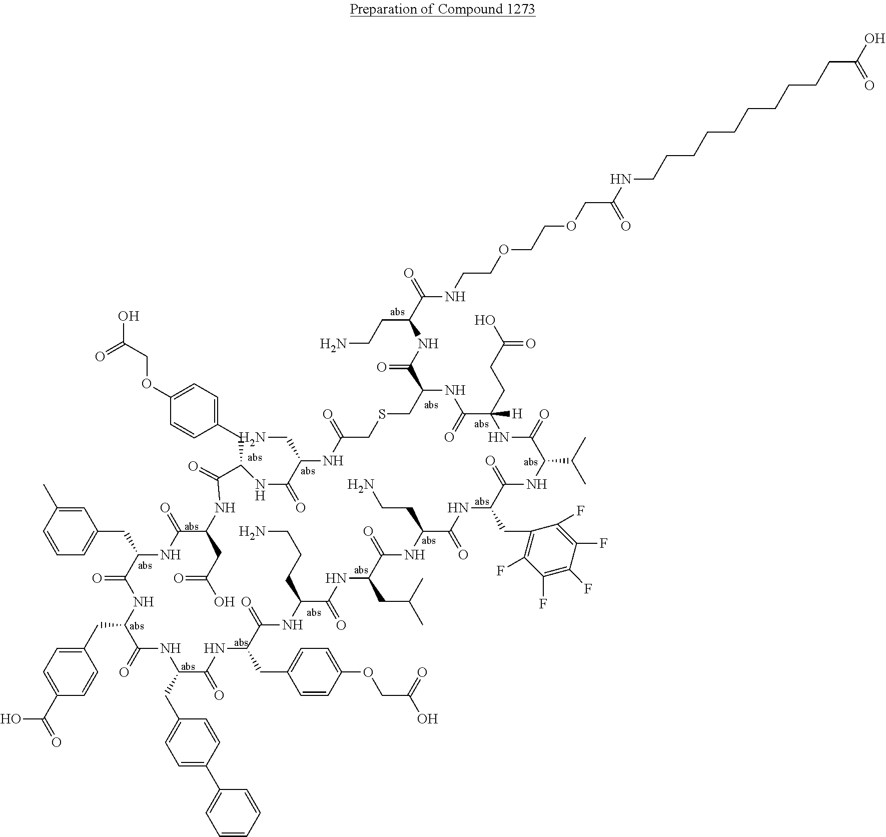 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3c(F)c(F)c(F)c(F)c3F)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1273 was prepared on a 50 μmol scale. The yield of the product was 10.6 mg, and its estimated purity by LCMS analysis was 100%. Analysis condition B: Retention time=1.55 min; ESI-MS(+) m/z [M+2H]²⁺: 1302.

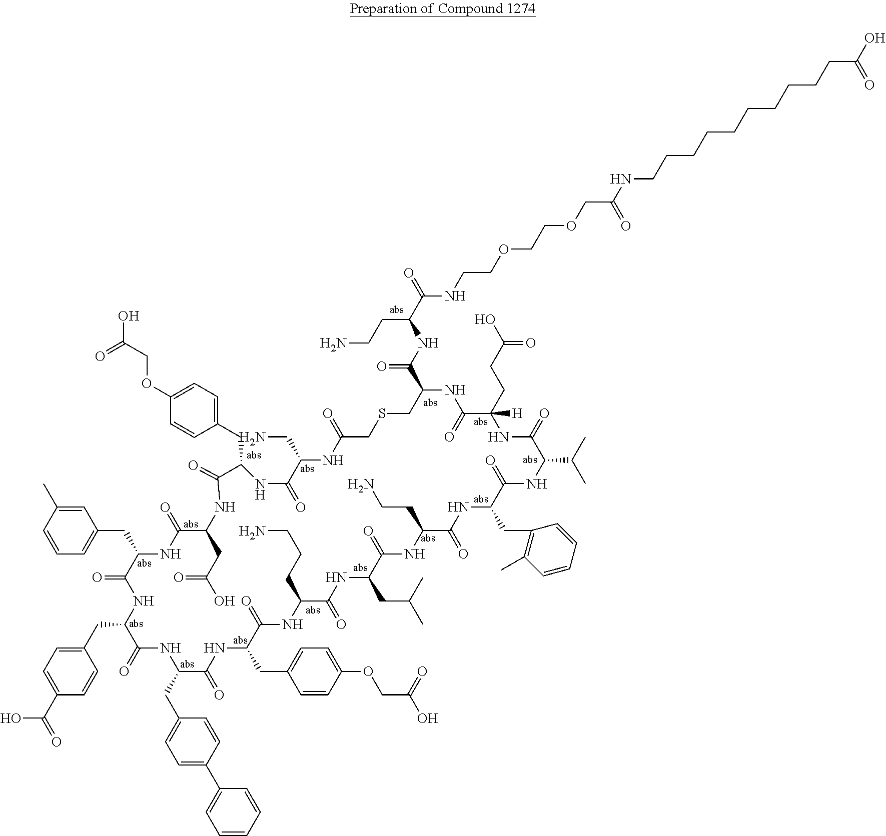 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3ccccc3C)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1274 was prepared on a 50 μmol scale. The yield of the product was 8.1 mg, and its estimated purity by LCMS analysis was 99.2%. Analysis condition A: Retention time=1.44 min; ESI-MS(+) m/z [M+2H]²⁺: 1264.

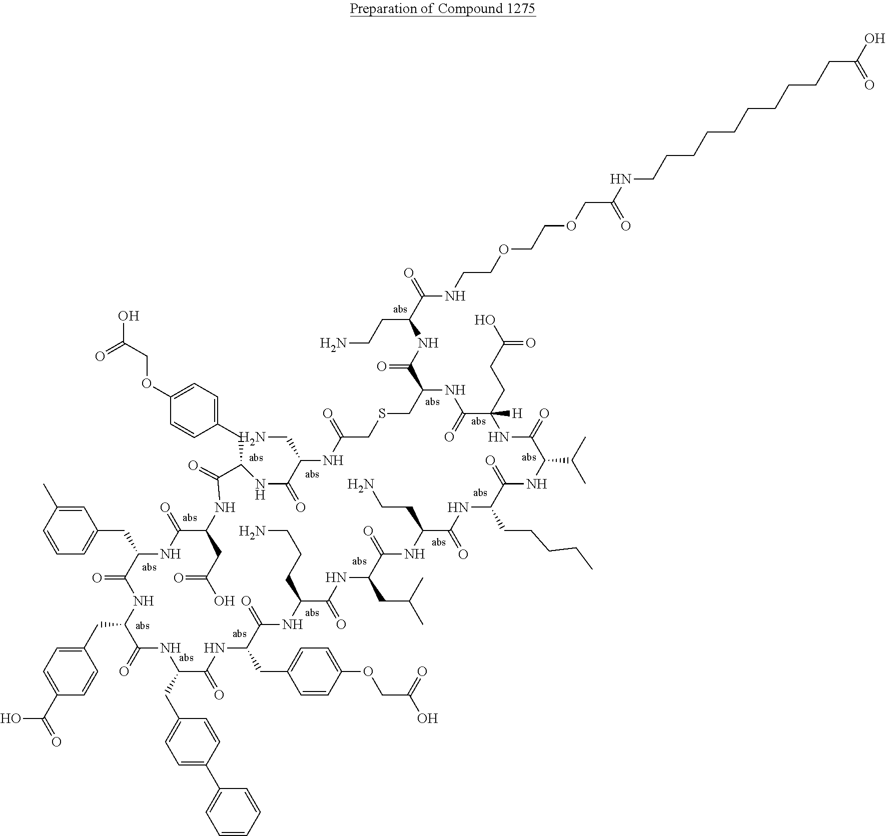 [SMILES: CCCCC[C@@H]1NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC1=O]

Compound 1275 was prepared on a 50 μmol scale. The yield of the product was 4.5 mg, and its estimated purity by LCMS analysis was 90.7%. Analysis condition B: Retention time=1.68 min; ESI-MS(+) m/z [M+2H]²⁺: 1247.

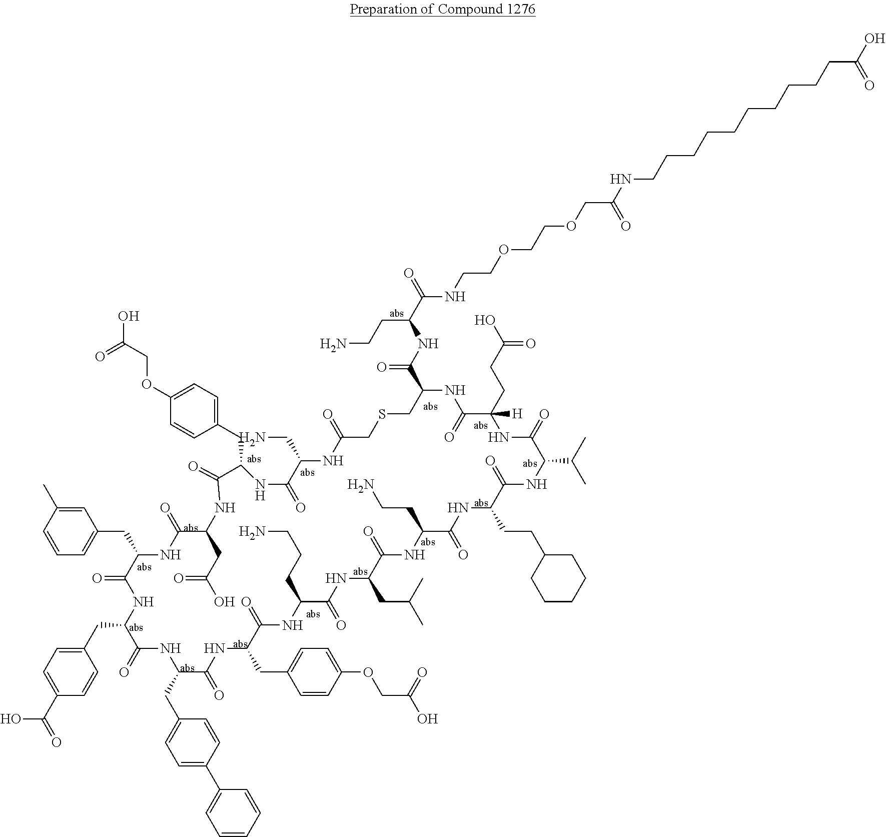 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CCC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1276 was prepared on a 50 μmol scale. The yield of the product was 4.9 mg, and its estimated purity by LCMS analysis was 100%. Analysis condition A: Retention time=1.45 min; ESI-MS(+) m/z [M+3H]³⁺: 845.

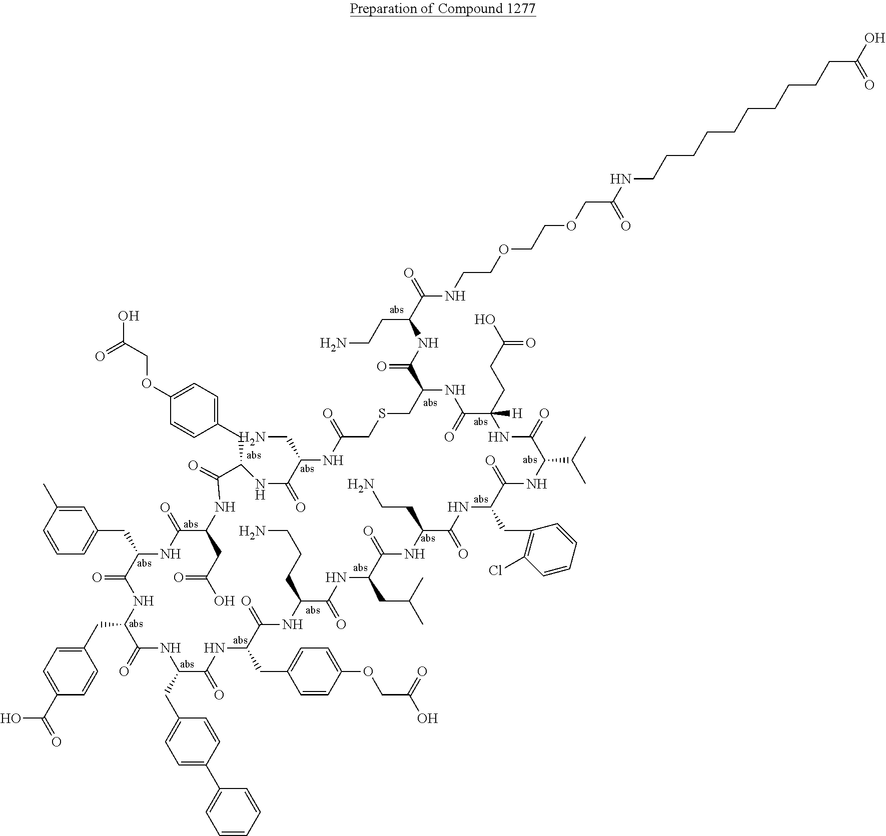 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](Cc3ccccc3Cl)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1277 was prepared on a 50 μmol scale. The yield of the product was 8.6 mg, and its estimated purity by LCMS analysis was 100%. Analysis condition A: Retention time=1.43 min; ESI-MS(+) m/z [M+2H]²⁺: 1273.9.

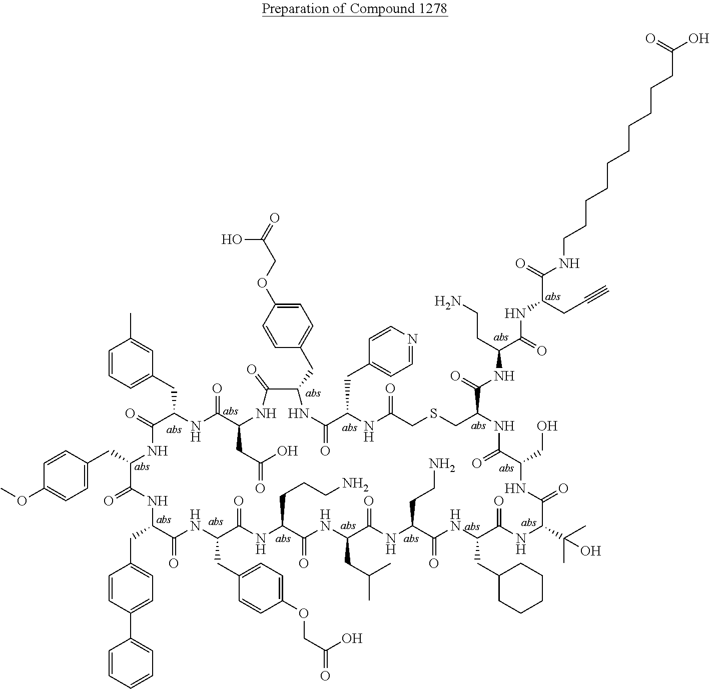 [SMILES: C#CC[C@H](NC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OC)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)NCCCCCCCCCCC(=O)O]

Compound 1278 was prepared on a 50 μmol scale. The yield of the product was 18 mg, and its estimated purity by LCMS analysis was 98.5%. Analysis condition A: Retention time=1.63 min; ESI-MS(+) m/z [M+2H]²⁺: 1245.5.

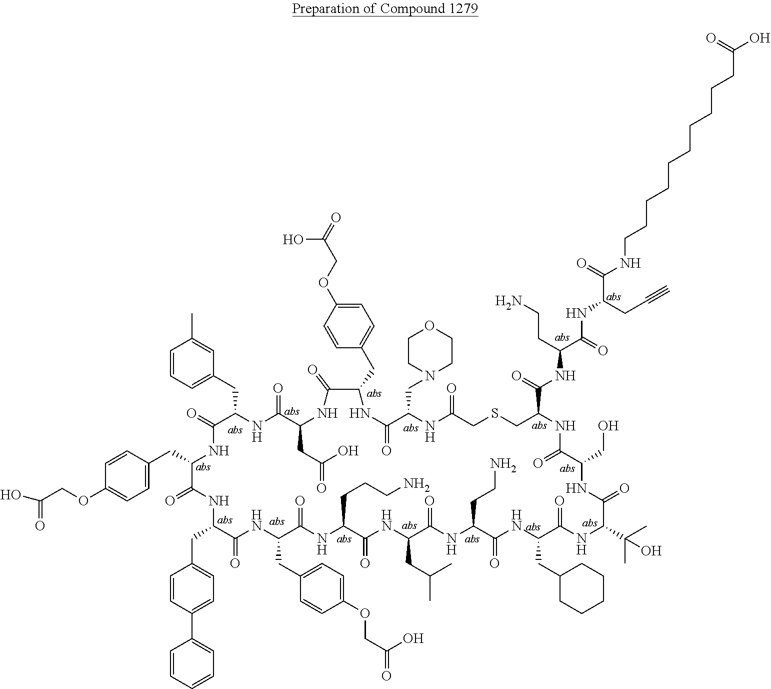 [SMILES: C#CC[C@H](NC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)NCCCCCCCCCCC(=O)O]

Compound 1279 was prepared on a 50 μmol scale. The yield of the product was 45.1 mg, and its estimated purity by LCMS analysis was 86.4%. Analysis condition A: Retention time=1.54 min; ESI-MS(+) m/z [M+2H]²⁺: 1272.

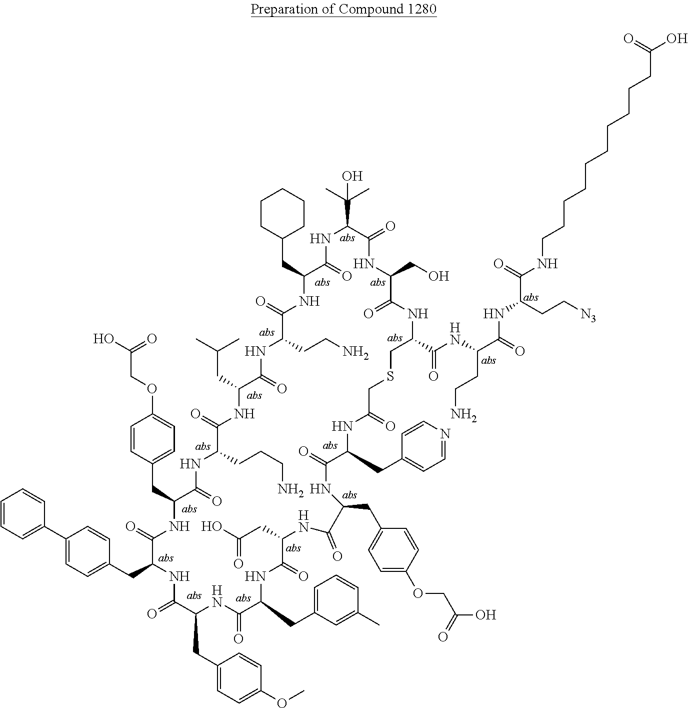 [SMILES: COc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)N[C@@H](CCN=[N+]=[N-])C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1280 was prepared on a 50 μmol scale. The yield of the product was 19.2 mg, and its estimated purity by LCMS analysis was 96.9%. Analysis condition A: Retention time=1.63 min; ESI-MS(+) m/z [M+2H]²⁺: 1261.2.

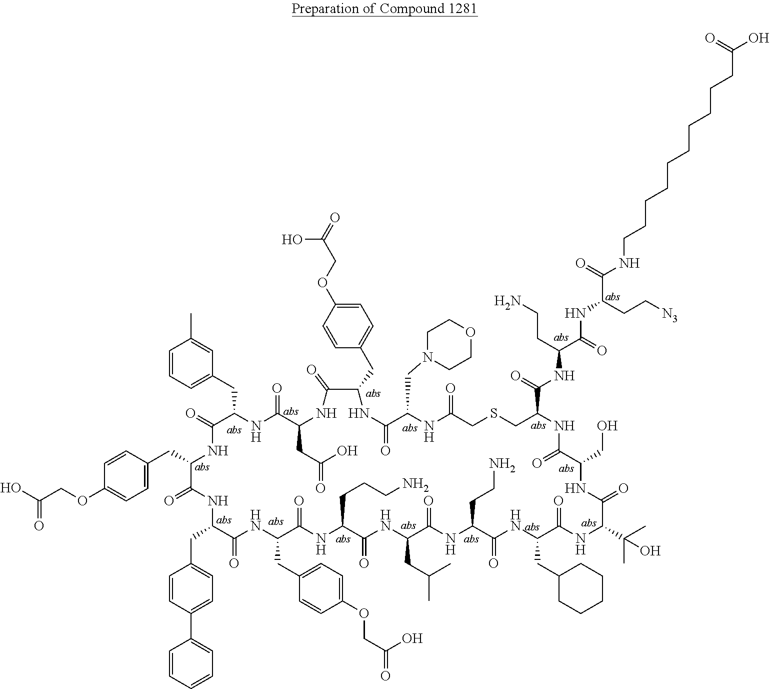 [SMILES: Cc1cccc(C[C@@H]2NC(=O)[C@H](CC(=O)O)NC(=O)[C@H](Cc3ccc(OCC(=O)O)cc3)NC(=O)[C@H](CN3CCOCC3)NC(=O)CSC[C@@H](C(=O)N[C@@H](CCN)C(=O)N[C@@H](CCN=[N+]=[N-])C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1281 was prepared on a 50 μmol scale. The yield of the product was 5.4 mg, and its estimated purity by LCMS analysis was 100%. Analysis condition A: Retention time=1.49 min; ESI-MS(+) m/z [M+2H]²⁺: 1286.9.

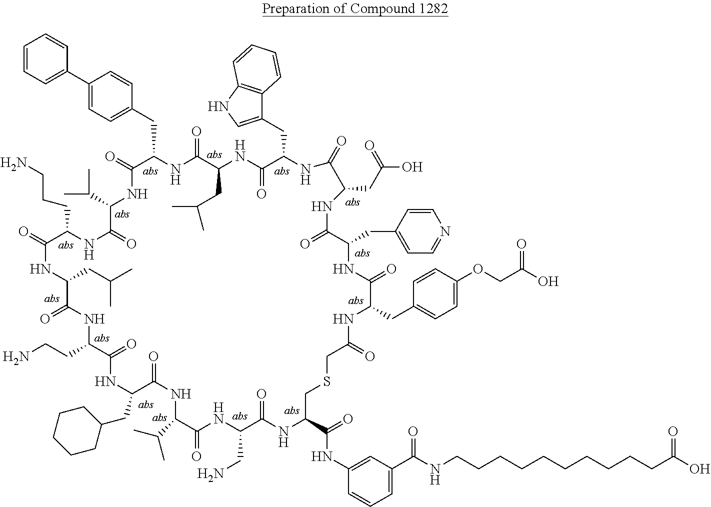 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2cccc(C(=O)NCCCCCCCCCCC(=O)O)c2)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1282 was prepared on a 50 μmol scale. The yield of the product was 3.8 mg, and its estimated purity by LCMS analysis was 85.2%. Analysis condition B: Retention time=1.55 min; ESI-MS(+) m/z [M+3H]³⁺: 746.1.

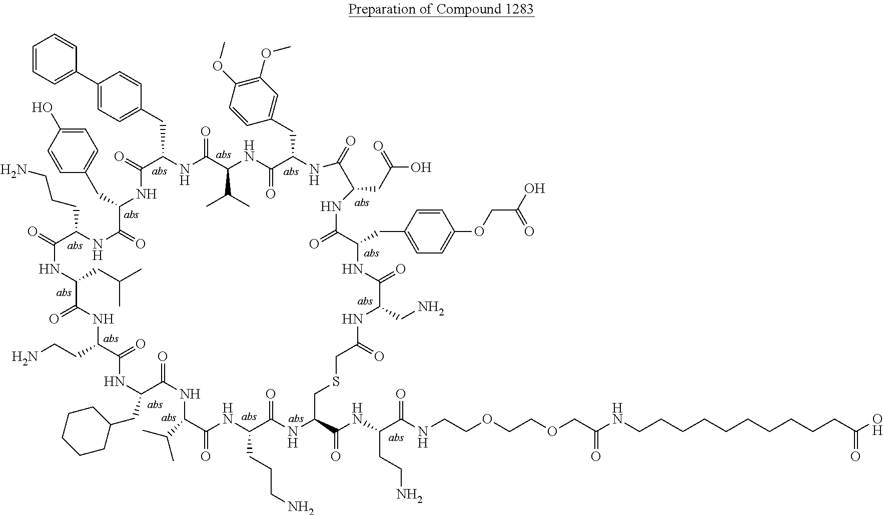 [SMILES: COc1ccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)cc1OC]

Compound 1283 was prepared on a 50 μmol scale. The yield of the product was 12.2 mg, and its estimated purity by LCMS analysis was 90.5%. Analysis condition A: Retention time=1.54 min; ESI-MS(+) m/z [M+2H]²⁺: 1200.2.

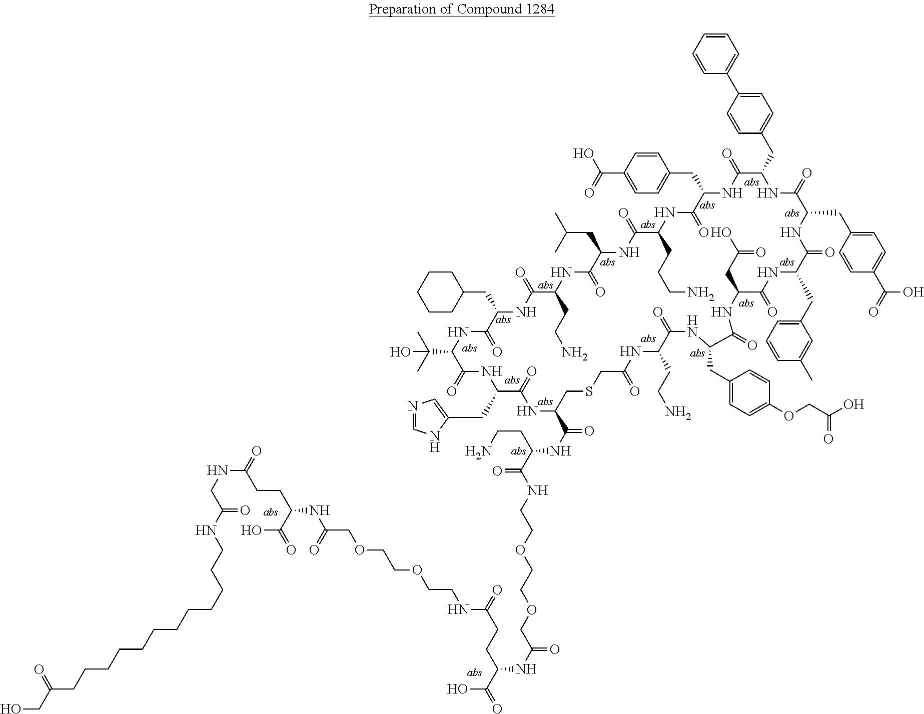 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)N[C@@H](CCC(=O)NCCOCCOCC(=O)N[C@@H](CCC(=O)NCC(=O)NCCCCCCCCCCCCC(=O)CO)C(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 1284 was prepared on a 50 μmol scale. The yield of the product was 19.4 mg, and its estimated purity by LCMS analysis was 100%. Analysis condition A: Retention time=1.56 min; ESI-MS(+) m/z [M+2H]²⁺: 1005.2.

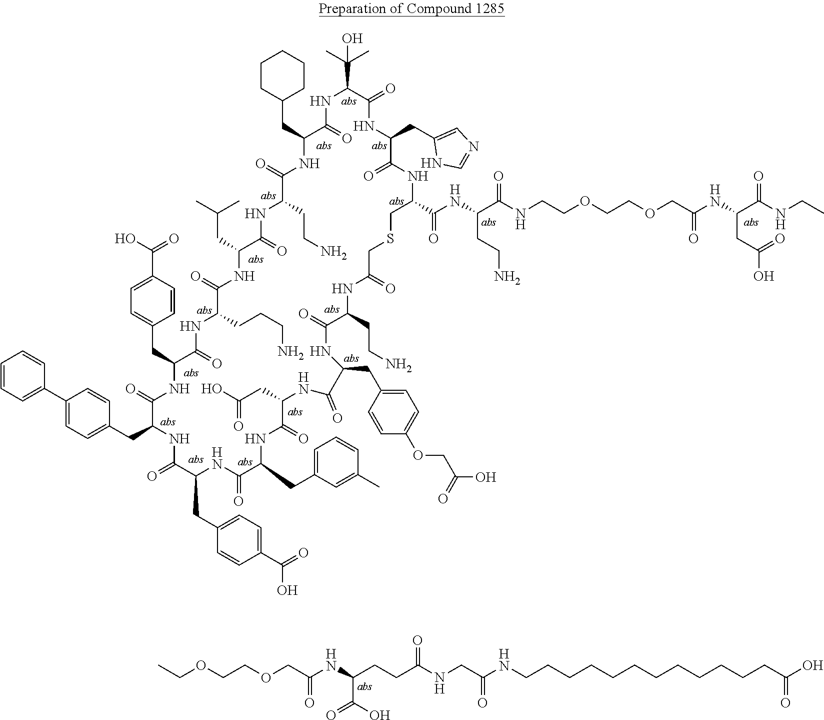

Compound 1285 was prepared on a 50 μmol scale. The yield of the product was 8.7 mg, and its estimated purity by LCMS analysis was 97.1%. Analysis condition B: Retention time=1.66 min; ESI-MS(+) m/z [M+2H]²⁺: 1001.1.

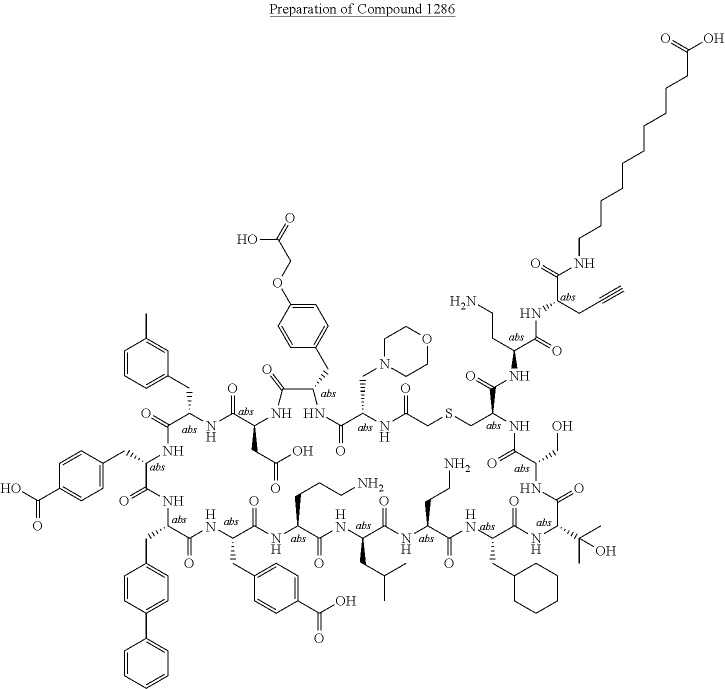 [SMILES: C#CC[C@H](NC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)NCCCCCCCCCCC(=O)O]

Compound 1286 was prepared on a 100 μmol scale. The yield of the product was 18.5 mg, and its estimated purity by LCMS analysis was 82.9%. Analysis condition A: Retention time=1.59 min; ESI-MS(+) m/z [M+2H]²⁺: 1241.3.

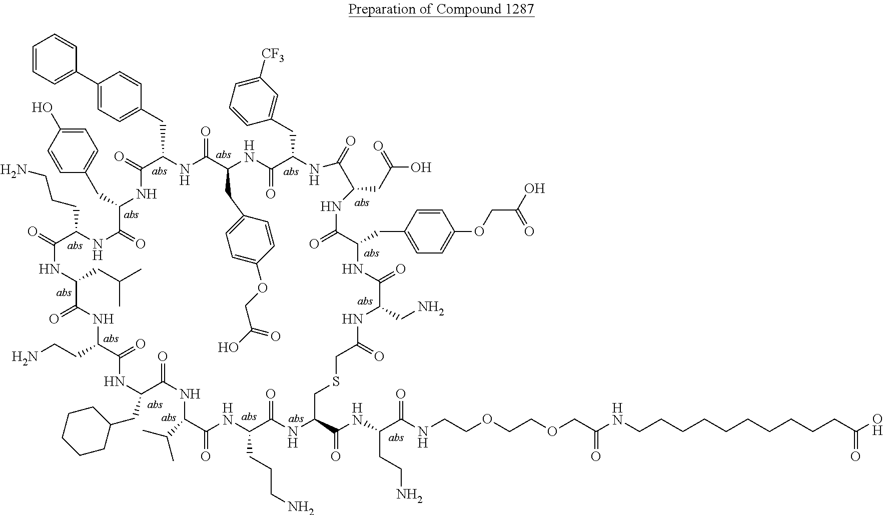 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1287 was prepared on a 50 μmol scale. The yield of the product was 5.2 mg, and its estimated purity by LCMS analysis was 91.9%. Analysis condition A: Retention time=1.64 min; ESI-MS(+) m/z [M+2H]²⁺: 1264.9.

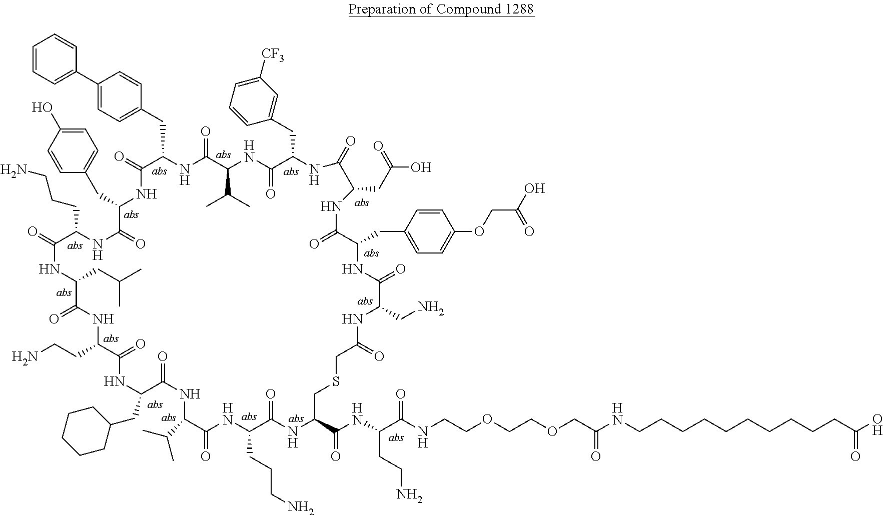 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1288 was prepared on a 50 μmol scale. The yield of the product was 17.8 mg, and its estimated purity by LCMS analysis was 95.4%. Analysis condition B: Retention time=1.62 min; ESI-MS(+) m/z [M+3H]³⁺: 803.2.

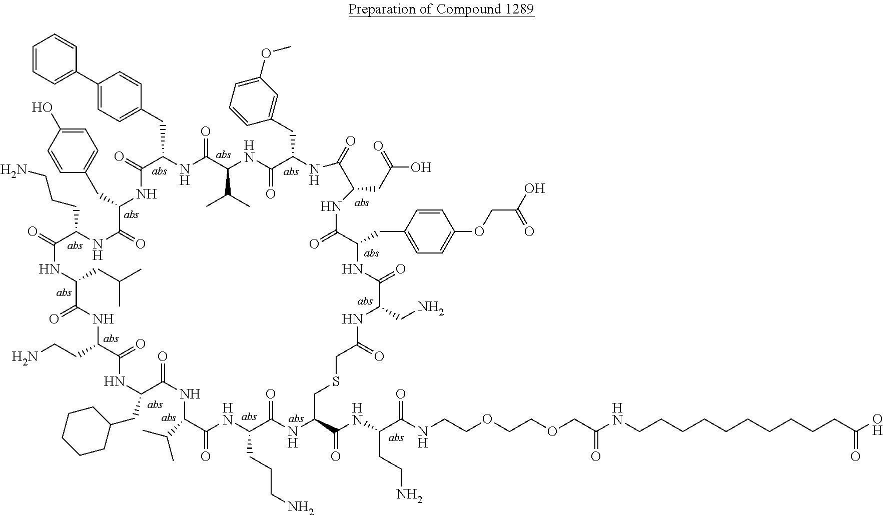 [SMILES: COc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 1289 was prepared on a 50 μmol scale. The yield of the product was 11.2 mg, and its estimated purity by LCMS analysis was 86.5%. Analysis condition B: Retention time=1.56 min; ESI-MS(+) m/z [M+2H]²⁺: 1185.2.

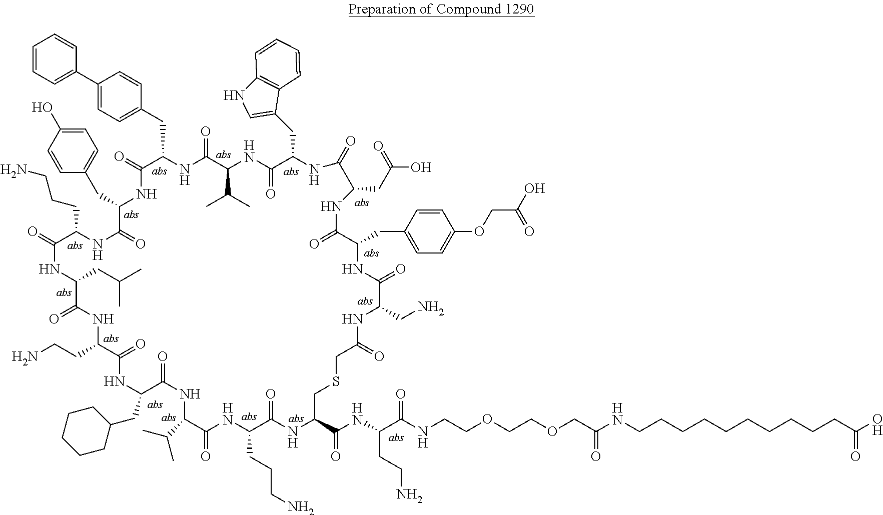 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1290 was prepared on a 50 μmol scale. The yield of the product was 17 mg, and its estimated purity by LCMS analysis was 95.6%. Analysis condition B: Retention time=1.5 min; ESI-MS(+) m/z [M+3H]³⁺: 793.2.

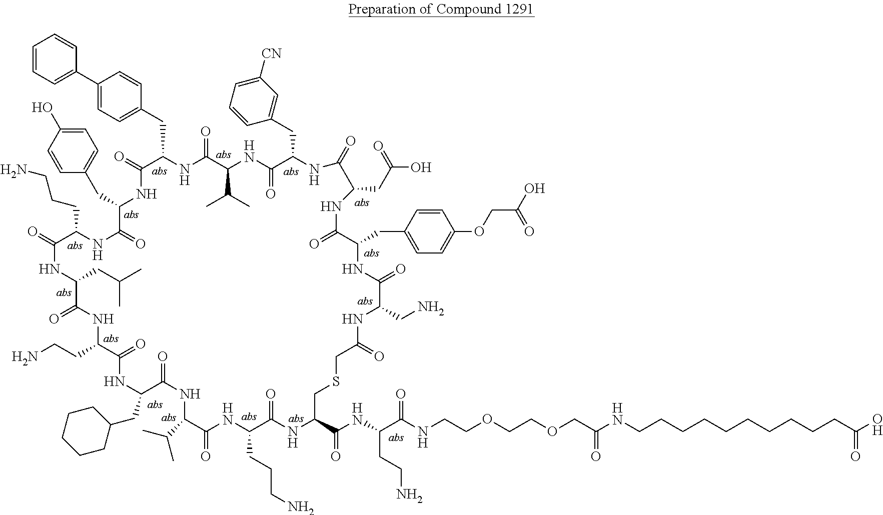 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2cccc(C#N)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1291 was prepared on a 50 μmol scale. The yield of the product was 8.5 mg, and its estimated purity by LCMS analysis was 91.7%. Analysis condition A: Retention time=1.56 min; ESI-MS(+) m/z [M+3H]³⁺: 789.1.

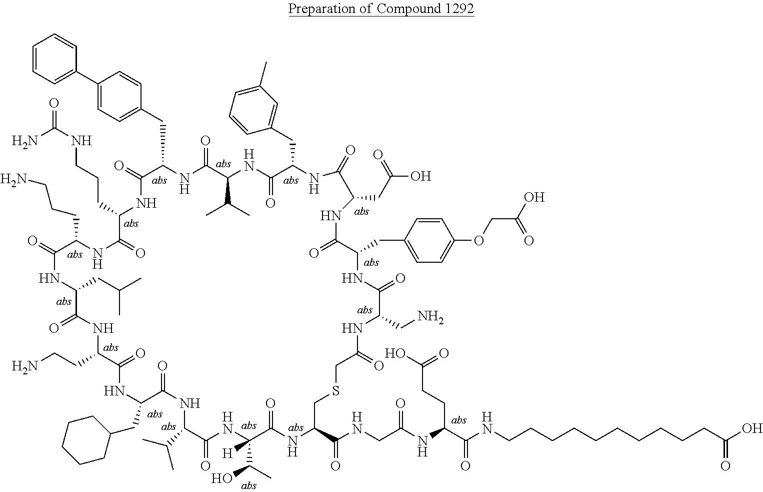 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1292 was prepared on a 50 μmol scale. The yield of the product was 8 mg, and its estimated purity by LCMS analysis was 87.2%. Analysis condition B: Retention time=1.6 min; ESI-MS(+) m/z [M+2H]²⁺: 1138.

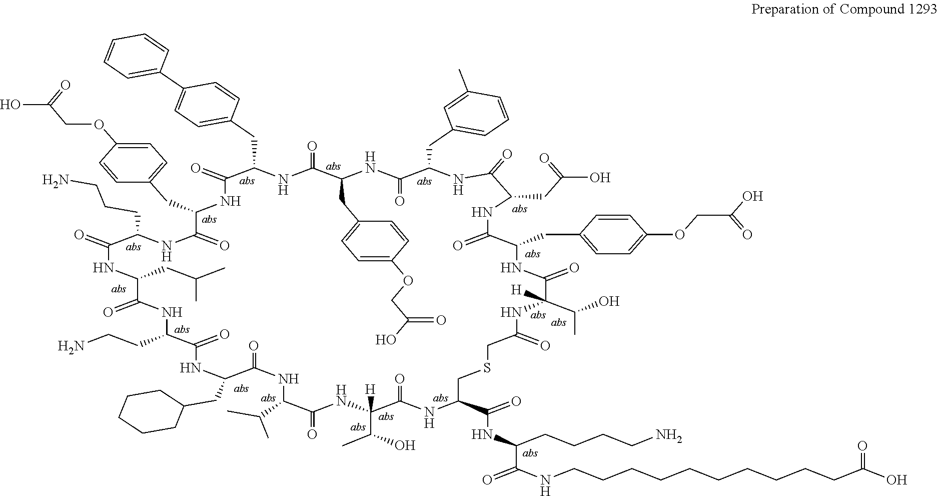 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1293 was prepared on a 50 μmol scale. The yield of the product was 4 mg, and its estimated purity by LCMS analysis was 92.2%. Analysis condition B: Retention time=1.62 min; ESI-MS(+) m/z [M+2H]²⁺: 1210.1.

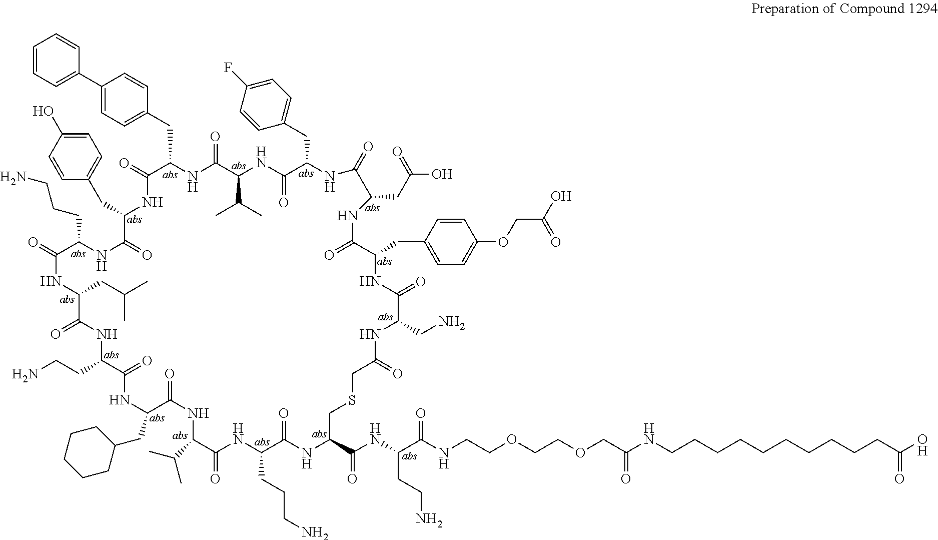 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2ccc(F)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1294 was prepared on a 50 μmol scale. The yield of the product was 4.3 mg, and its estimated purity by LCMS analysis was 77.3%. Analysis condition B: Retention time=1.58 min; ESI-MS(+) m/z [M+2H]²⁺: 1179.1.

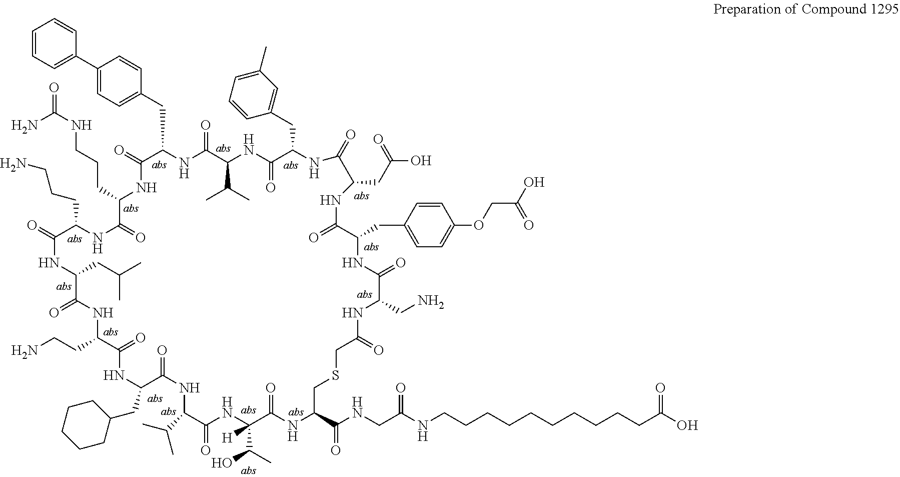 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1295 was prepared on a 50 μmol scale. The yield of the product was 15.2 mg, and its estimated purity by LCMS analysis was 90.4%. Analysis condition B: Retention time=1.59 min; ESI-MS(+) m/z [M+3H]³⁺: 716.1.

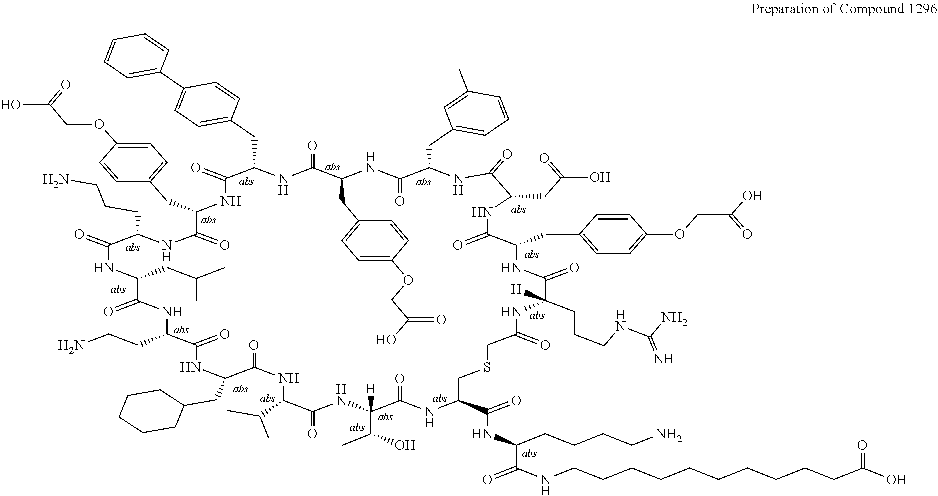 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1296 was prepared on a 50 μmol scale. The yield of the product was 1.4 mg, and its estimated purity by LCMS analysis was 100%. Analysis condition A: Retention time=1.54 min; ESI-MS(+) m/z [M+2H]²⁺: 1237.

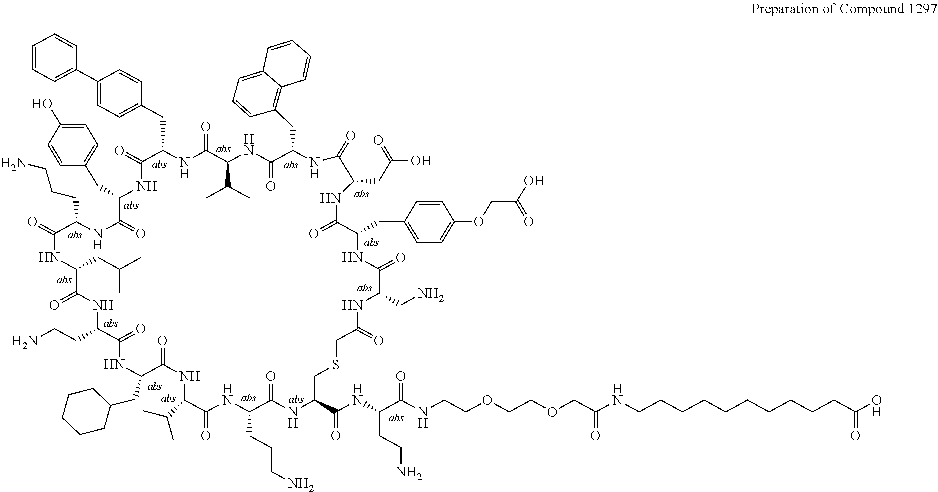 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2cccc3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1297 was prepared on a 50 μmol scale. The yield of the product was 16.1 mg, and its estimated purity by LCMS analysis was 100%. Analysis condition B: Retention time=1.62 min; ESI-MS(+) m/z [M+3H]³⁺: 797.3.

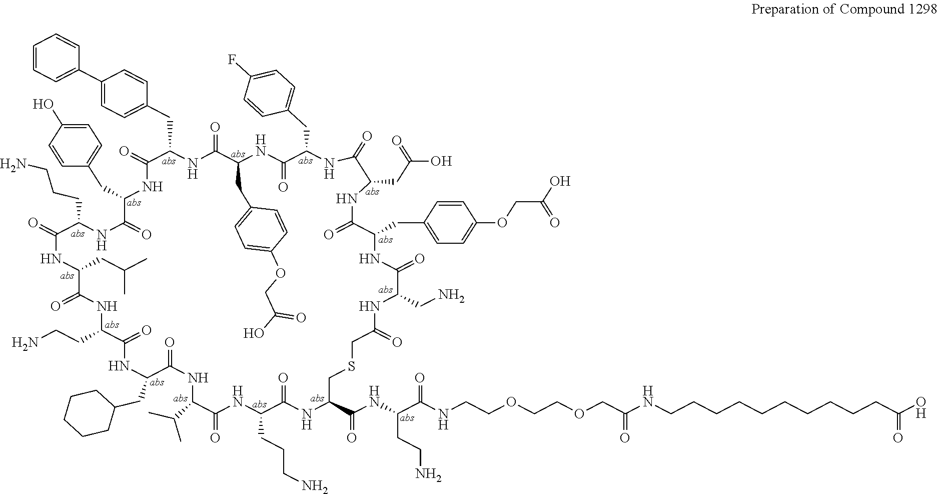 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2ccc(F)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1298 was prepared on a 50 μmol scale. The yield of the product was 9.1 mg, and its estimated purity by LCMS analysis was 97.1%. Analysis condition A: Retention time=1.57 min; ESI-MS(+) m/z [M+2H]²⁺: 1240.1.

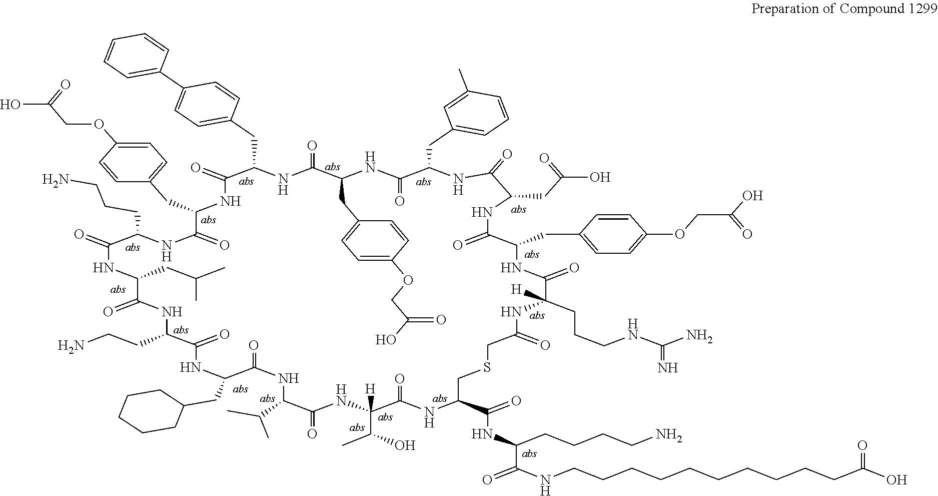 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1299 was prepared on a 50 μmol scale. The yield of the product was 2.8 mg, and its estimated purity by LCMS analysis was 70.5%. Analysis condition B: Retention time=1.53 min; ESI-MS(+) m/z [M+3H]³⁺: 815.4.

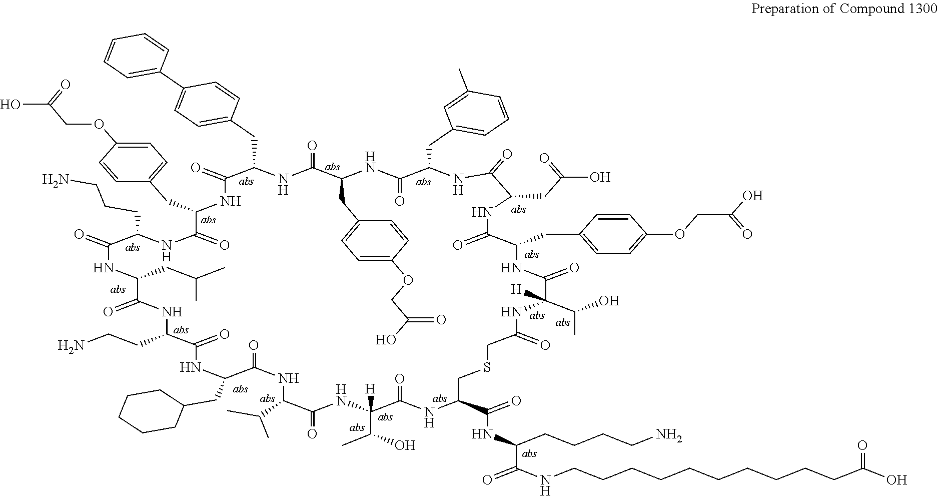 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1300 was prepared on a 50 μmol scale. The yield of the product was 3.7 mg, and its estimated purity by LCMS analysis was 91.4%. Analysis condition A: Retention time=1.35 min; ESI-MS(+) m/z [M+3H]³⁺: 786.5.

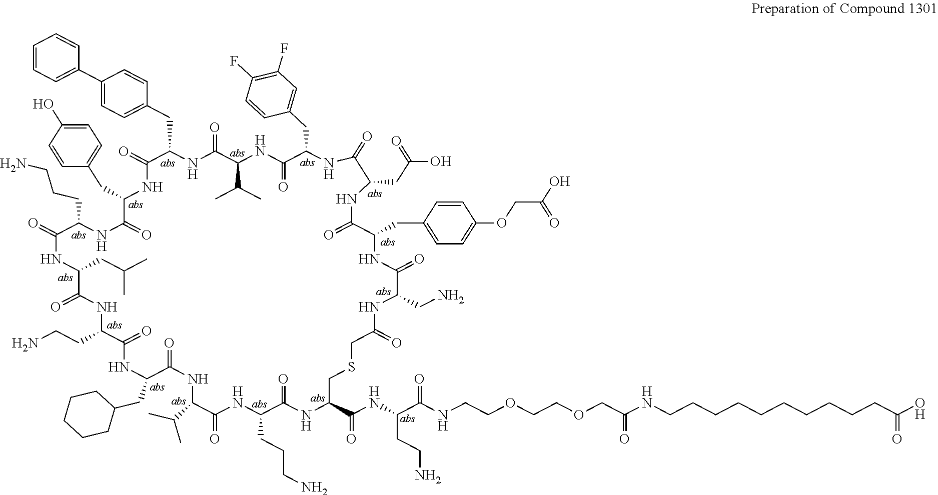 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2ccc(F)c(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1301 was prepared on a 50 μmol scale. The yield of the product was 20.4 mg, and its estimated purity by LCMS analysis was 97.1%. Analysis condition A: Retention time=1.68 min; ESI-MS(+) m/z [M+2H]²⁺: 1188.1.

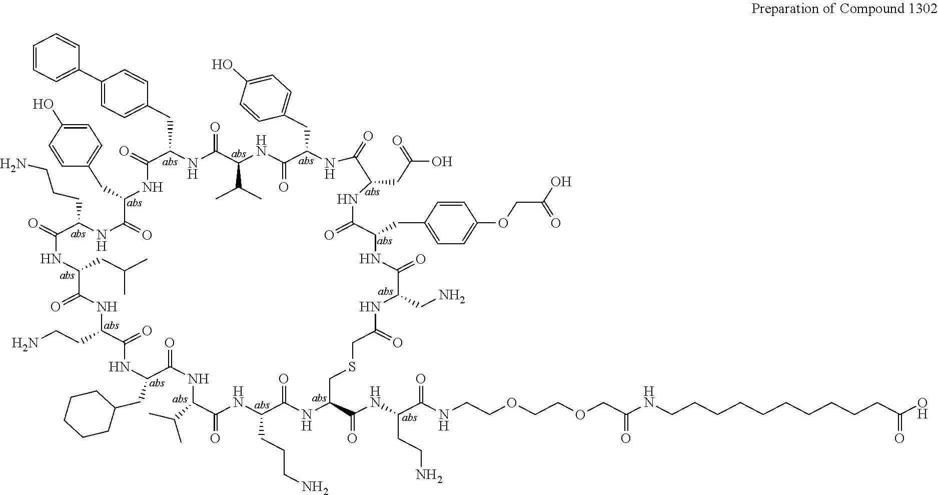 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2ccc(O)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1302 was prepared on a 50 μmol scale. The yield of the product was 17.3 mg, and its estimated purity by LCMS analysis was 91.2%. Analysis condition A: Retention time=1.43 min; ESI-MS(+) m/z [M+2H]²⁺: 1177.9.

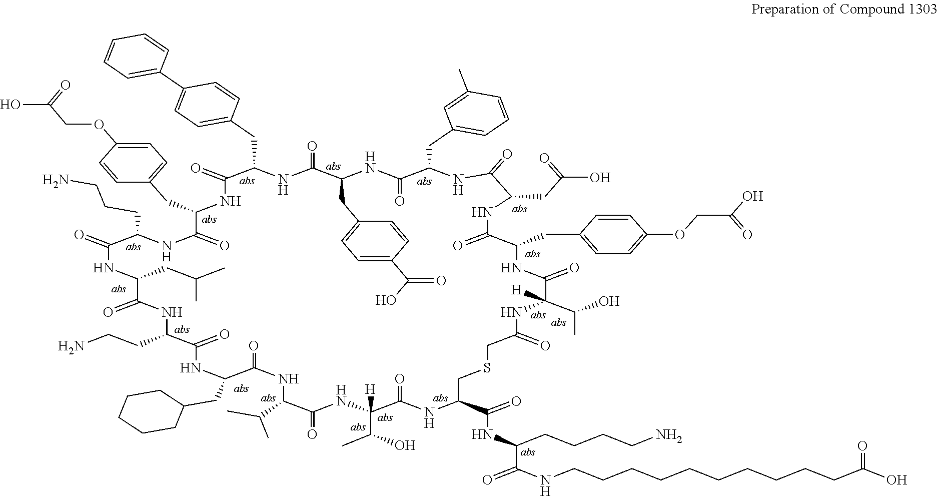 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 1303 was prepared on a 50 μmol scale. The yield of the product was 7 mg, and its estimated purity by LCMS analysis was 90.9%. Analysis condition B: Retention time=1.65 min; ESI-MS(+) m/z [M+2H]²⁺: 796.9.

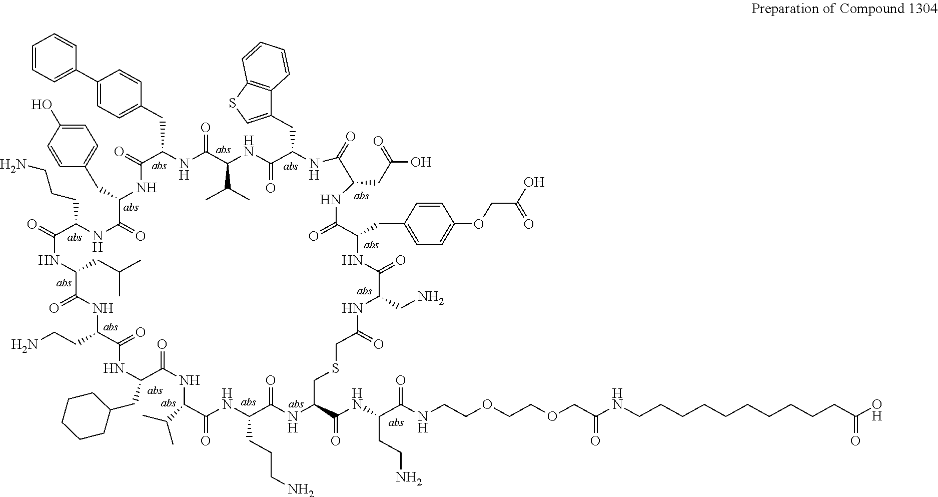 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2csc3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1304 was prepared on a 50 μmol scale. The yield of the product was 19.8 mg, and its estimated purity by LCMS analysis was 94.7%. Analysis condition B: Retention time=1.61 min; ESI-MS(+) m/z [M+2H]²⁺: 1198.1.

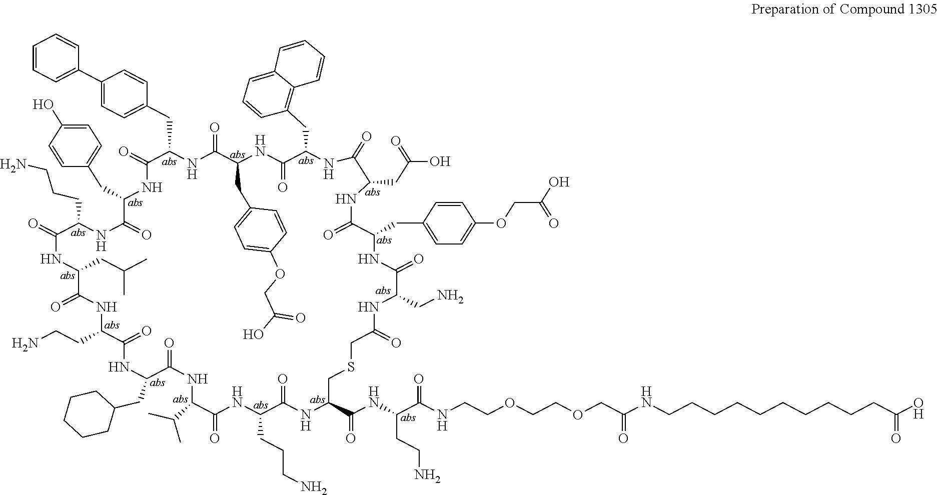 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2cccc3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1305 was prepared on a 50 μmol scale. The yield of the product was 2.8 mg, and its estimated purity by LCMS analysis was 90.5%. Analysis condition B: Retention time=1.62 min; ESI-MS(+) m/z [M+2H]²⁺: 1256.1.

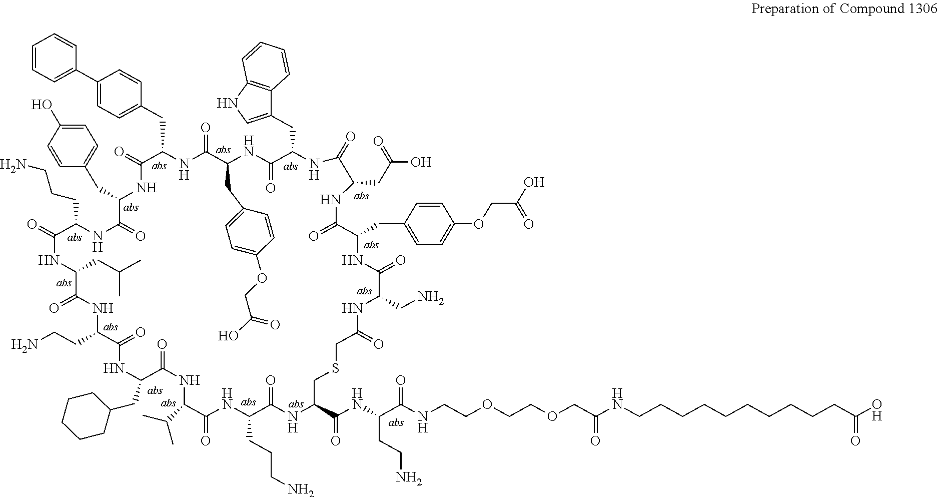 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1306 was prepared on a 50 μmol scale. The yield of the product was 7 mg, and its estimated purity by LCMS analysis was 91.8%. Analysis condition B: Retention time=1.49 min; ESI-MS(+) m/z [M+2H]²⁺: 1251.

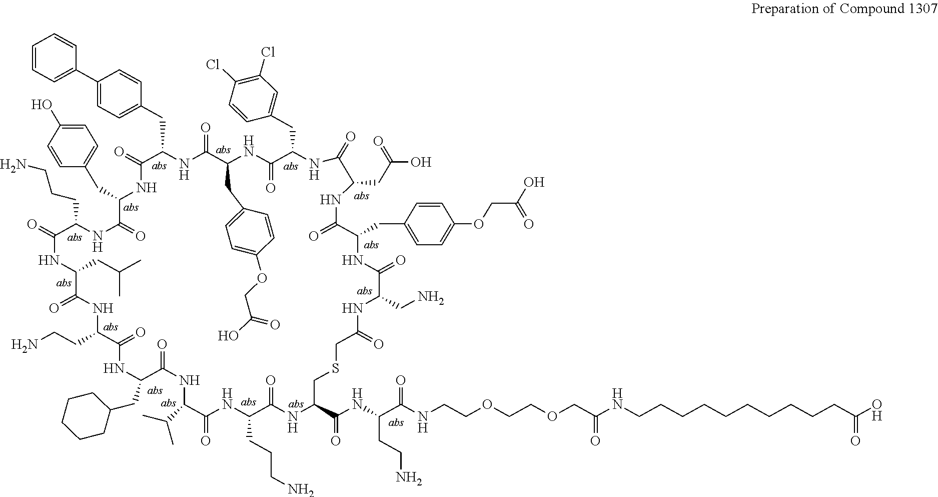 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2ccc(Cl)c(Cl)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1307 was prepared on a 50 μmol scale. The yield of the product was 6.5 mg, and its estimated purity by LCMS analysis was 91.3%. Analysis condition A: Retention time=1.63 min; ESI-MS(+) m/z [M+3H]³⁺: 843.8.

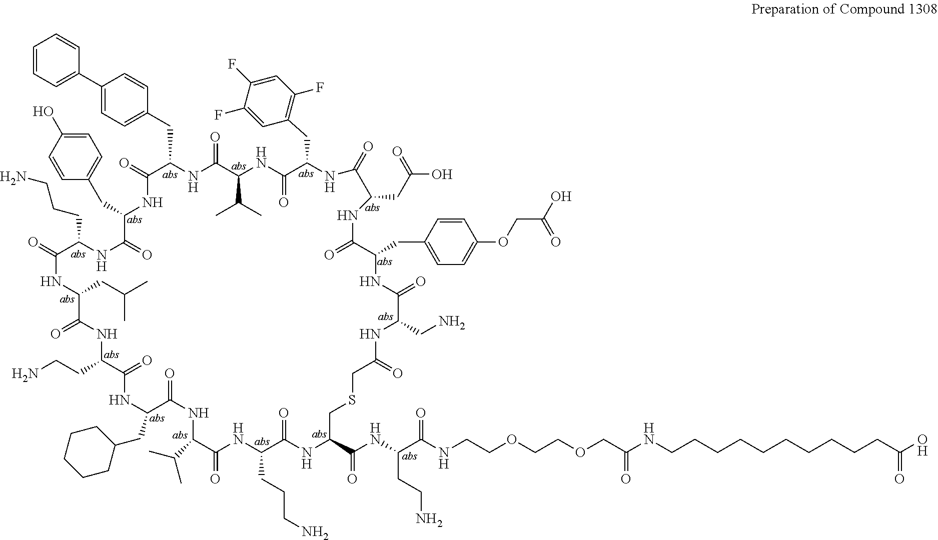 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2cc(F)c(F)cc2F)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1308 was prepared on a 50 μmol scale. The yield of the product was 2.5 mg, and its estimated purity by LCMS analysis was 95.8%. Analysis condition A: Retention time=1.71 min; ESI-MS(+) m/z [M+3H]³⁺: 798.1.

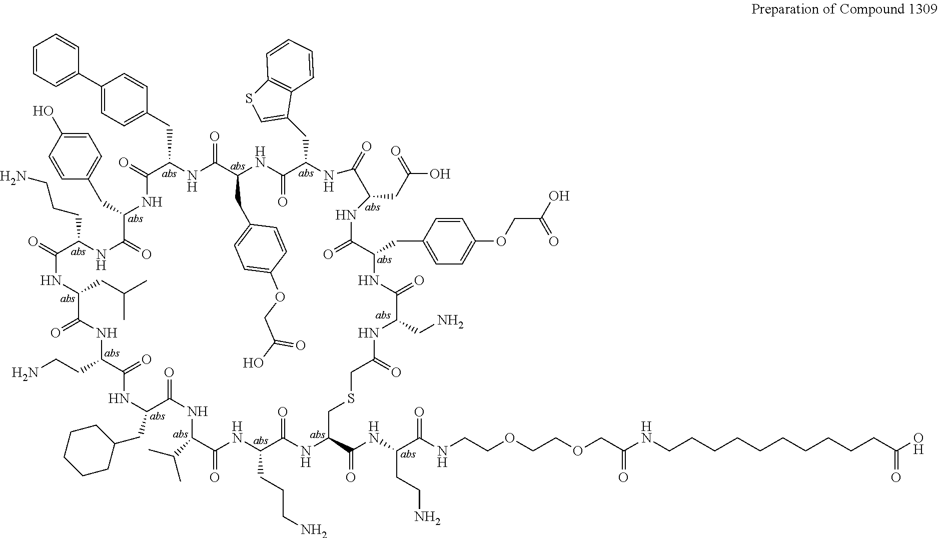 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2csc3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1309 was prepared on a 50 μmol scale. The yield of the product was 5.8 mg, and its estimated purity by LCMS analysis was 90%. Analysis condition A: Retention time=1.62 min; ESI-MS(+) m/z [M+2H]²⁺: 1259.

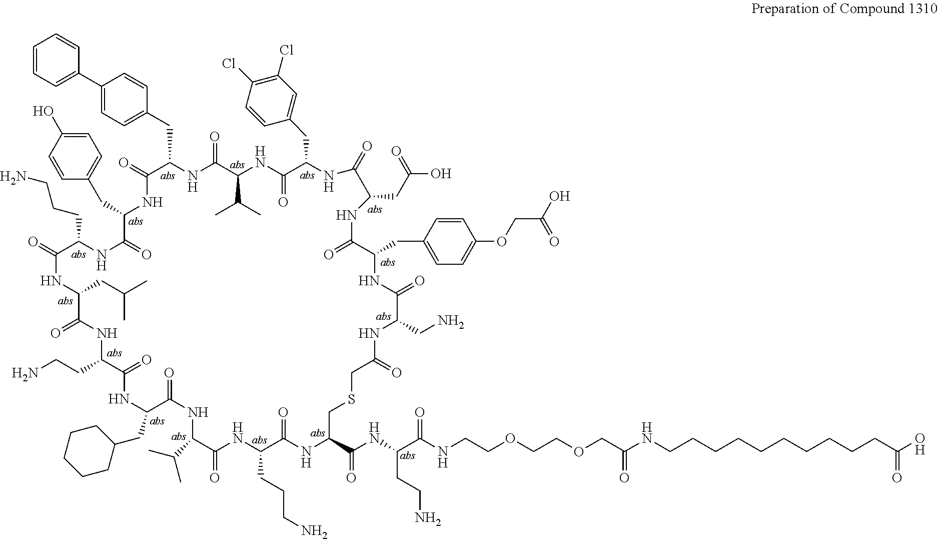 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2ccc(Cl)c(Cl)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1310 was prepared on a 50 μmol scale. The yield of the product was 5.2 mg, and its estimated purity by LCMS analysis was 91.7%. Analysis condition B: Retention time=1.64 min; ESI-MS(+) m/z [M+3H]³⁺: 803.1.

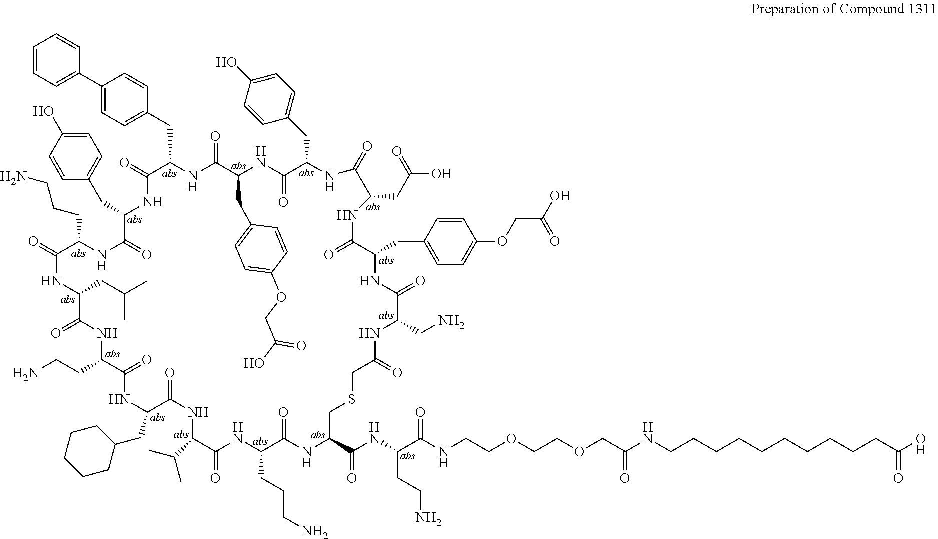 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2ccc(O)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1311 was prepared on a 50 μmol scale. The yield of the product was 7.2 mg, and its estimated purity by LCMS analysis was 90.9%. Analysis condition A: Retention time=1.46 min; ESI-MS(+) m/z [M+2H]²⁺: 1239.

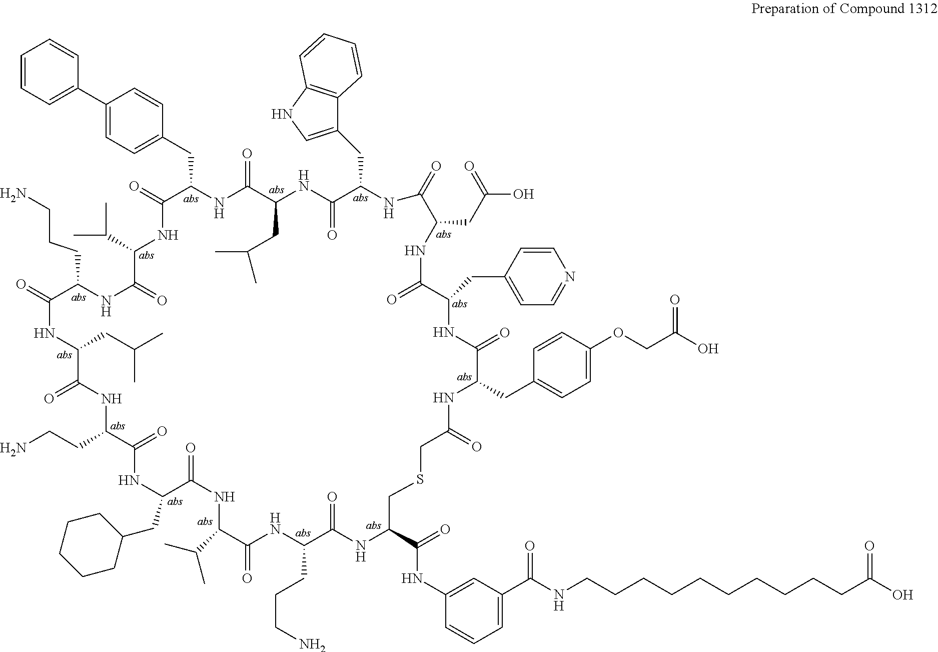 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)Nc2cccc(C(=O)NCCCCCCCCCCC(=O)O)c2)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1312 was prepared on a 50 μmol scale. The yield of the product was 2.1 mg, and its estimated purity by LCMS analysis was 95.6%. Analysis condition A: Retention time=1.87 min; ESI-MS(+) m/z [M+3H]³⁺: 755.2.

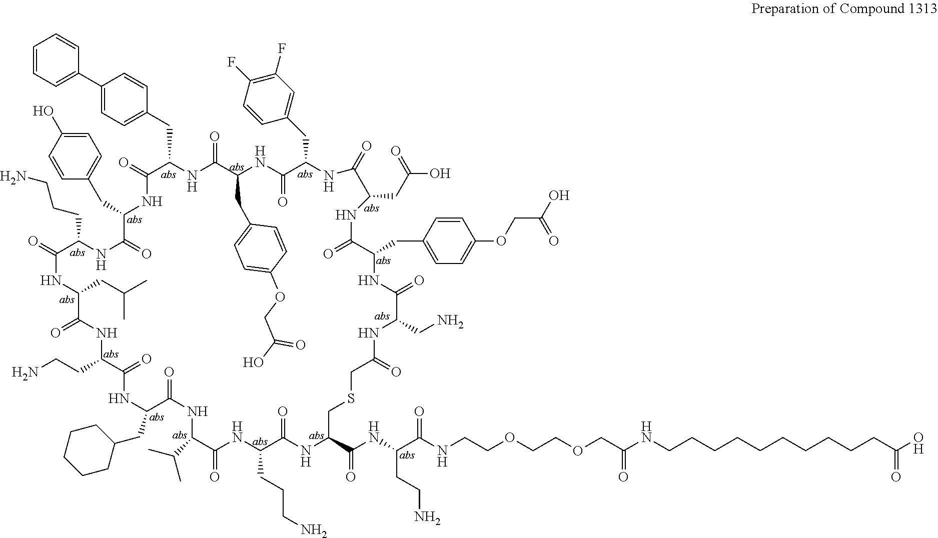 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(OCC(=O)O)cc2)NC(=O)[C@H](Cc2ccc(F)c(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 1313 was prepared on a 50 μmol scale. The yield of the product was 5.8 mg, and its estimated purity by LCMS analysis was 93%. Analysis condition B: Retention time=1.54 min; ESI-MS(+) m/z [M+2H]²⁺: 1248.9.

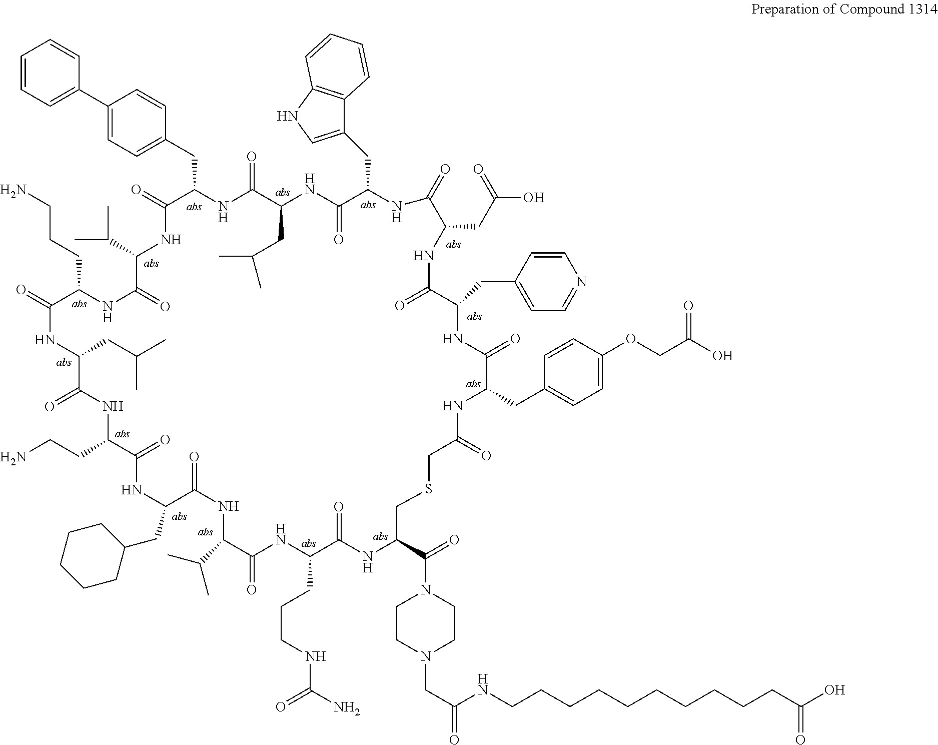 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N2CCN(CC(=O)NCCCCCCCCCCC(=O)O)CC2)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1314 was prepared on a 50 μmol scale. The yield of the product was 3 mg, and its estimated purity by LCMS analysis was 97.5%. Analysis condition A: Retention time=1.82 min; ESI-MS(+) m/z [M+3H]³⁺: 772.3.

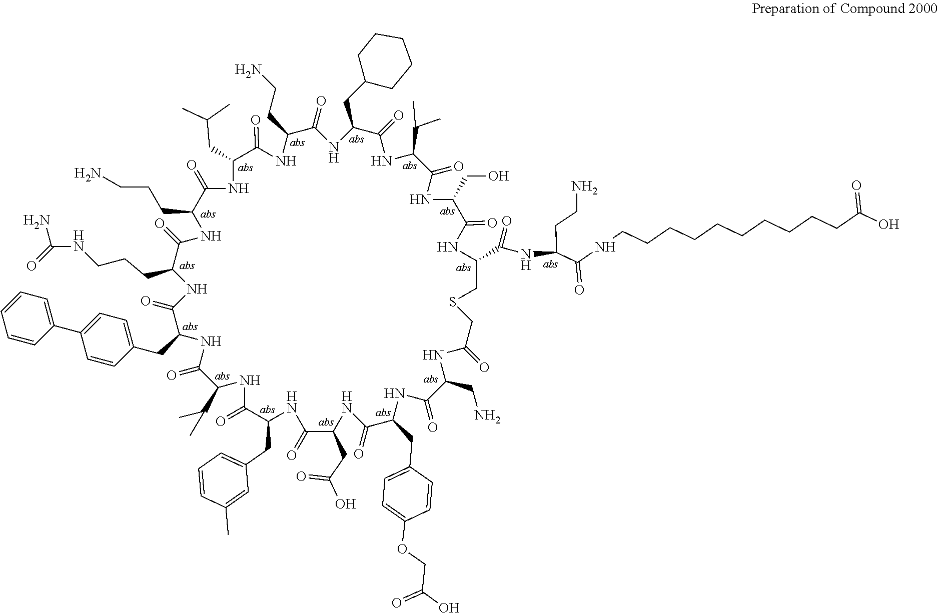 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2000 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 18.5 mg, and its estimated purity by LCMS analysis was 91.4%. Analysis condition 1: Retention time=1.53 min; ESI-MS(+) m/z [M+2H]²⁺: 1088.1.

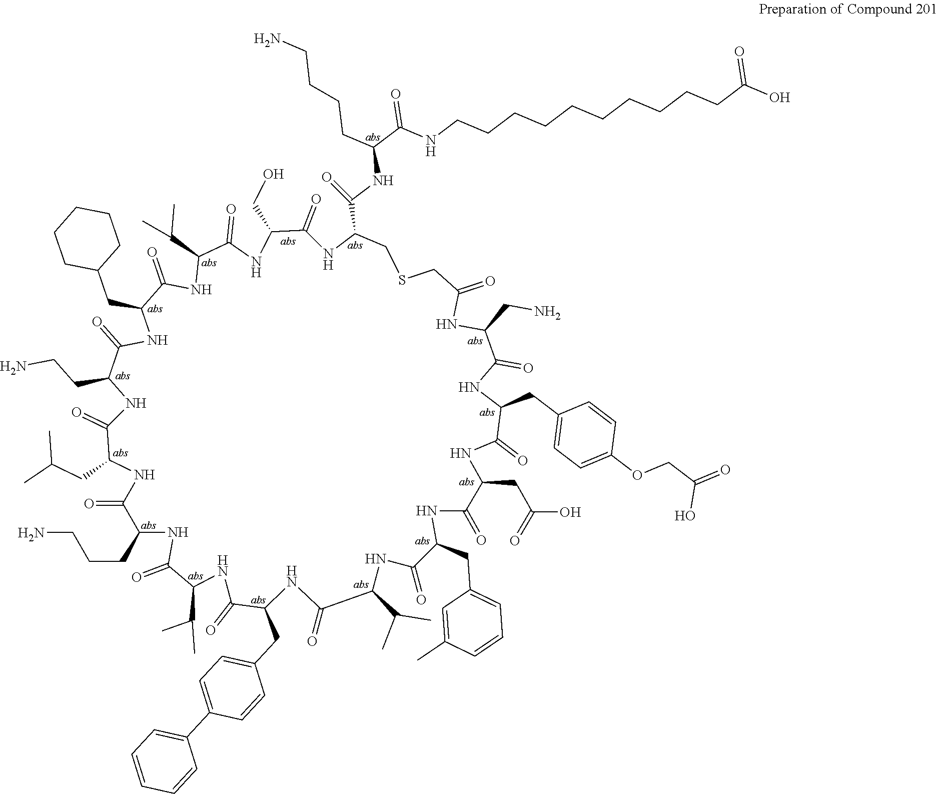 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2001 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.8 mg, and its estimated purity by LCMS analysis was 93.3%. Analysis condition 2: Retention time=1.77 min; ESI-MS(+) m/z [M+2H]²⁺: 1073.

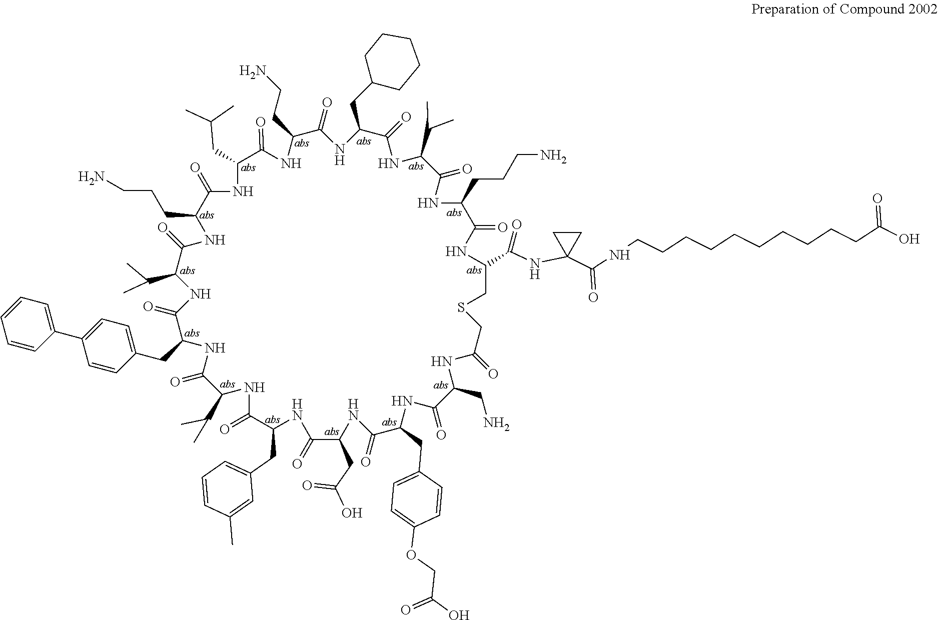

Compound 2002 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 19.8 mg, and its estimated purity by LCMS analysis was 92.2%. Analysis condition 2: Retention time=1.59 min; ESI-MS(+) m/z [M+2H]²⁺: 1064.2.

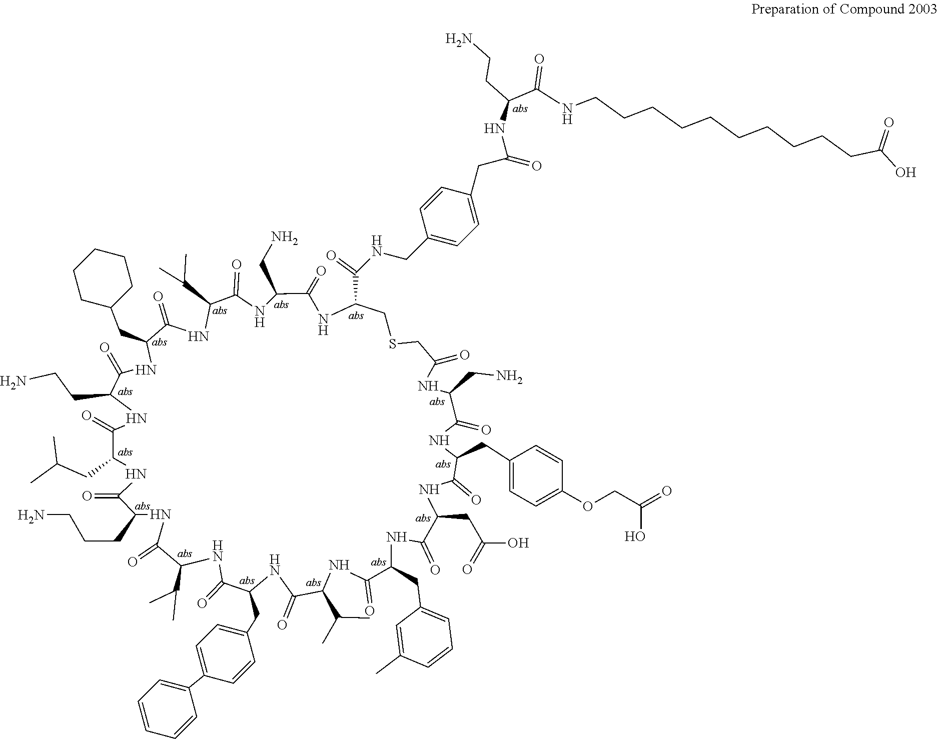 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCc3ccc(CC(=O)N[C@@H](CCN)C(=O)NCCCCCCCCCCC(=O)O)cc3)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2003 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 2-(4-(((((9H-fluoren-9-yl) methoxy) carbonyl)amino)methyl)phenyl) acetic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 11.4 mg, and its estimated purity by LCMS analysis was 97.3%. Analysis condition 2: Retention time=1.62 min; ESI-MS(+) m/z [M+2H]²⁺: 1132.2.

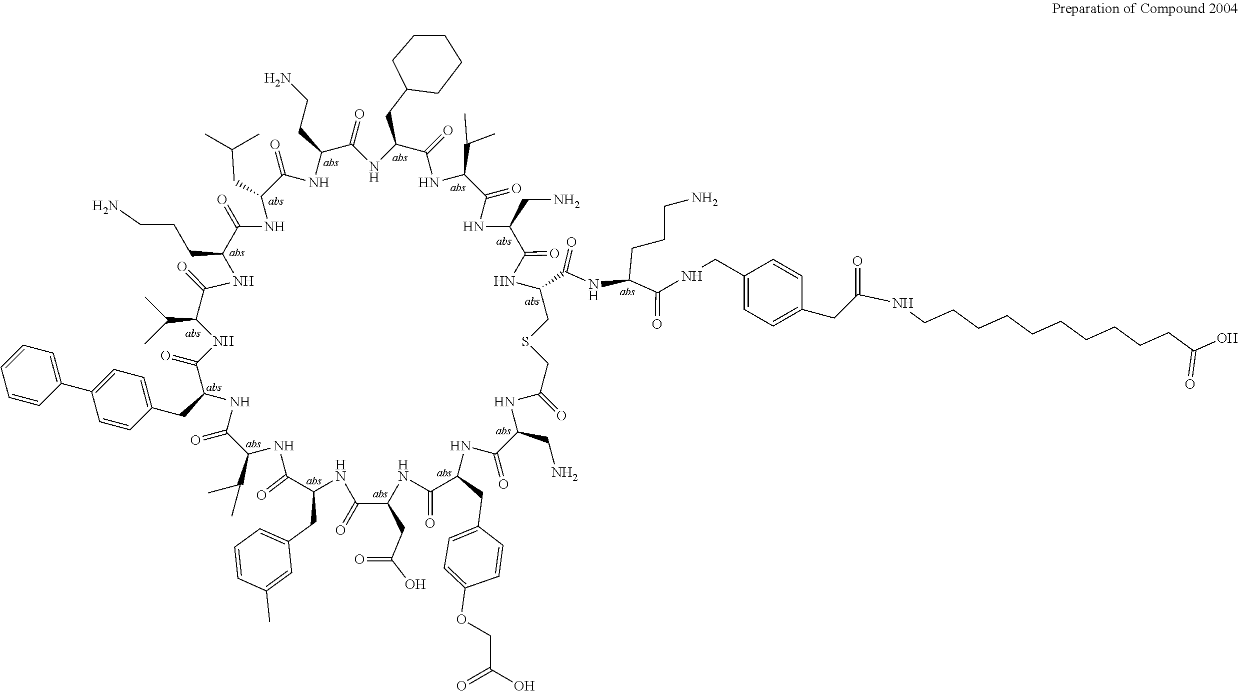 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)NCc3ccc(CC(=O)NCCCCCCCCCCC(=O)O)cc3)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2004 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 2-(4-(((((9H-fluoren-9-yl) methoxy) carbonyl)amino)methyl)phenyl) acetic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15 mg, and its estimated purity by LCMS analysis was 90.4%. Analysis condition 2: Retention time=1.75 min; ESI-MS(+) m/z [M+2H]²⁺: 1139.

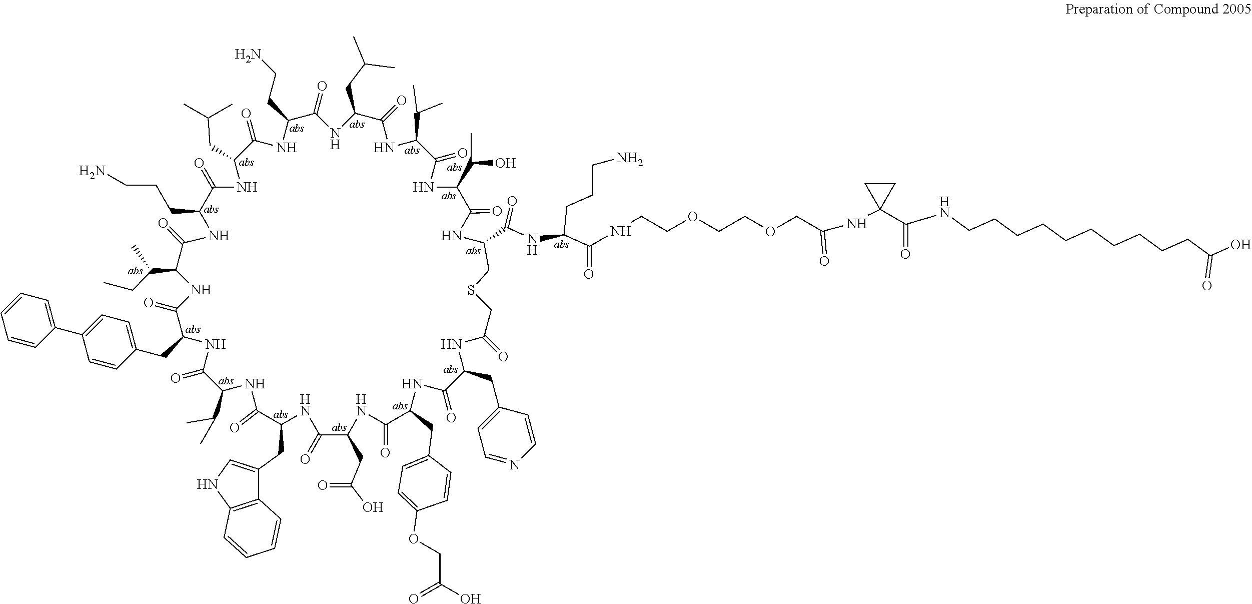 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NC2(C(=O)NCCCCCCCCCCC(=O)O)CC2)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2005 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6 mg, and its estimated purity by LCMS analysis was 91.7%. Analysis condition 1: Retention time=1.48 min; ESI-MS(+) m/z [M+3H]³⁺: 812.2.

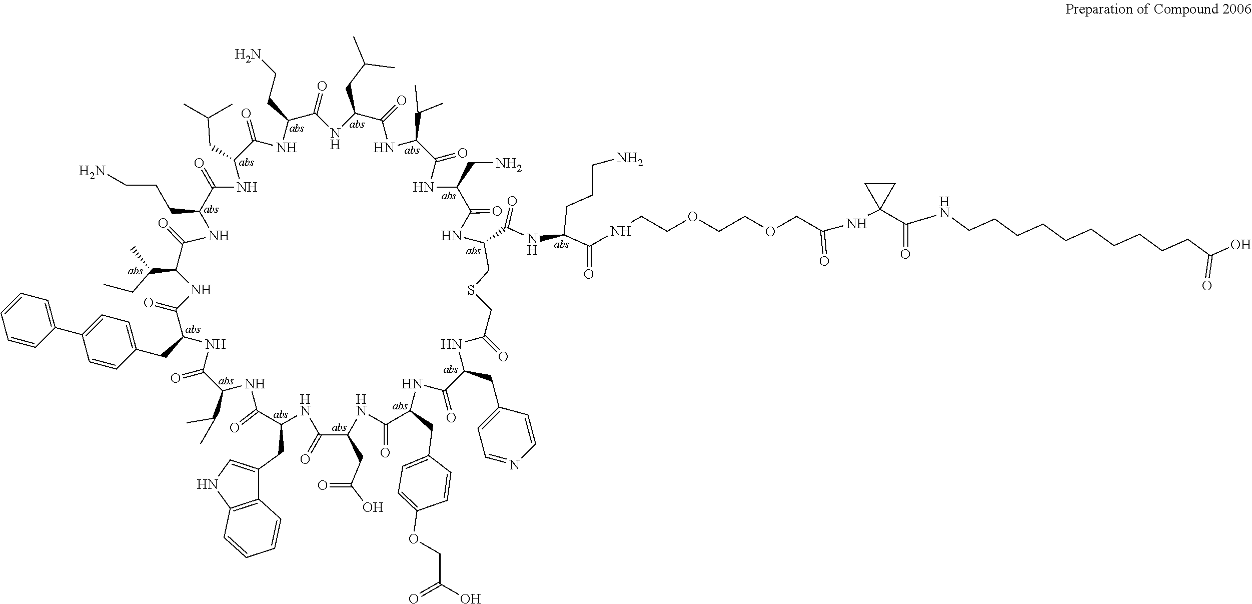 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NC2(C(=O)NCCCCCCCCCCC(=O)O)CC2)NC(=O)[C@H](CN)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2006 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.9 mg, and its estimated purity by LCMS analysis was 94.9%. Analysis condition 1: Retention time=1.43 min; ESI-MS(+) m/z [M+2H]²⁺: 1210.1.

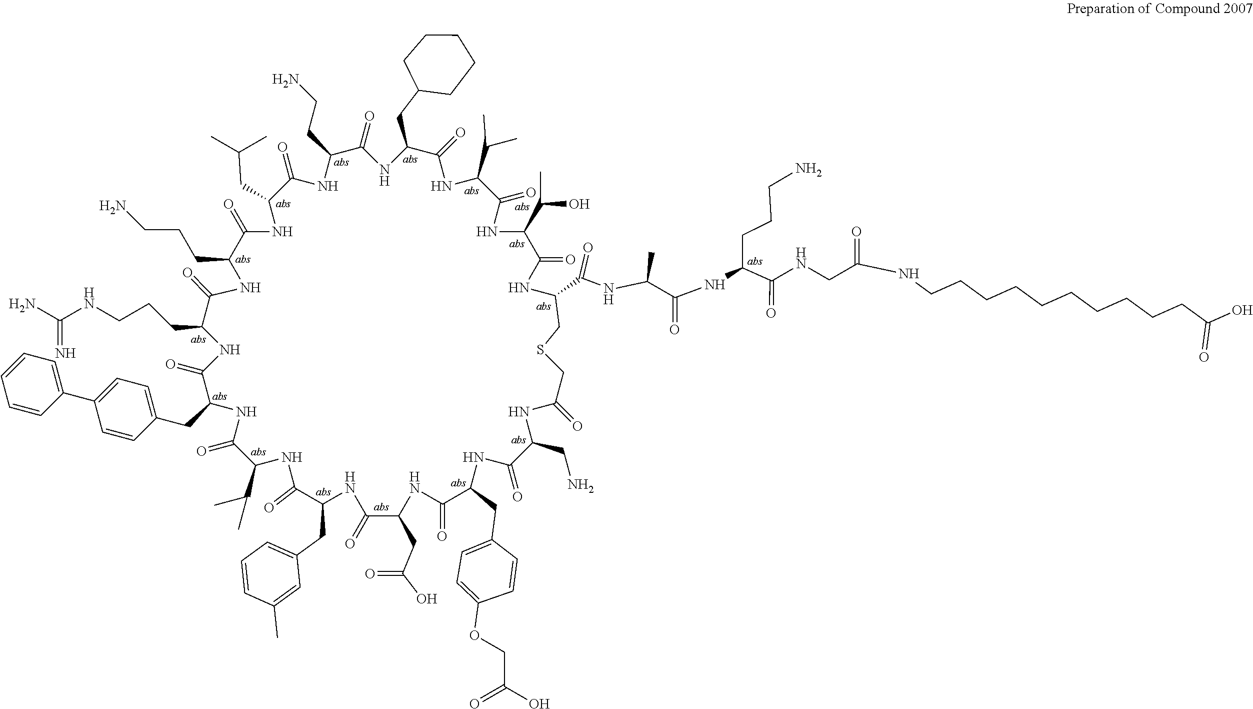 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCCN)C(=O)NCC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 2007 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.6 mg, and its estimated purity by LCMS analysis was 98.4%. Analysis condition 1: Retention time=1.57 min; ESI-MS(+) m/z [M+3H]³⁺: 778.

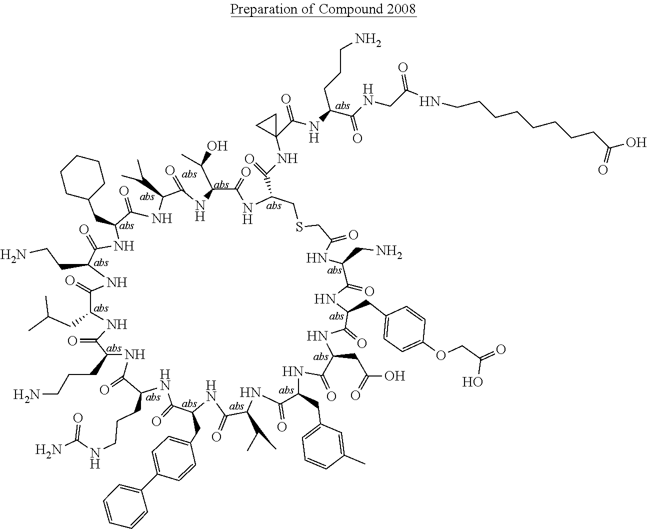 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NC3(C(=O)N[C@@H](CCCN)C(=O)NCC(=O)NCCCCCCCCC(=O)O)CC3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2008 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.6 mg, and its estimated purity by LCMS analysis was 91.7%. Analysis condition 1: Retention time=1.59 min; ESI-MS(+) m/z [M+2H]²⁺: 1171.6.

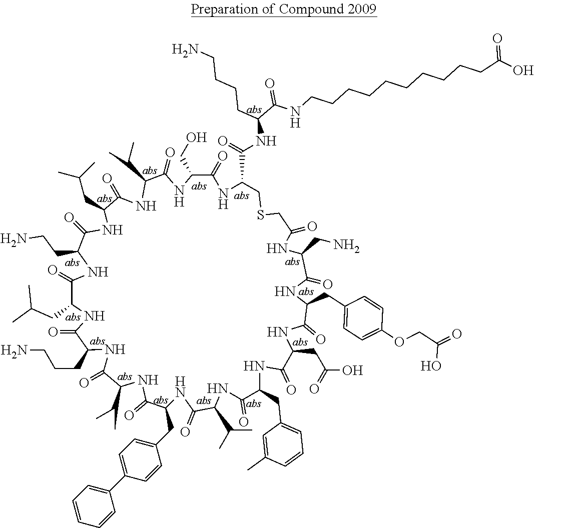 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2009 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 4.9 mg, and its estimated purity by LCMS analysis was 95.5%. Analysis condition 1: Retention time=1.39 min; ESI-MS(+) m/z [M+3H]³⁺: 702.2.

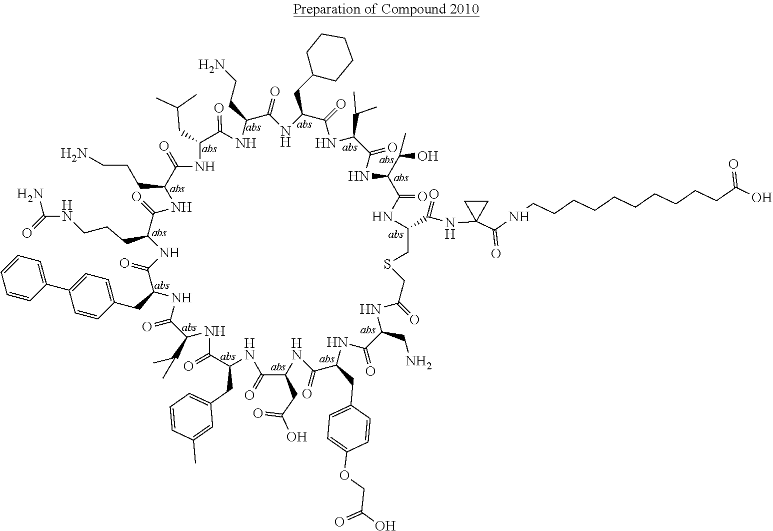 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NC3(C(=O)NCCCCCCCCCCC(=O)O)CC3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2010 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 29.2 mg, and its estimated purity by LCMS analysis was 95.2%. Analysis condition 2: Retention time=1.67 min; ESI-MS(+) m/z [M+3H]³⁺: 724.5.

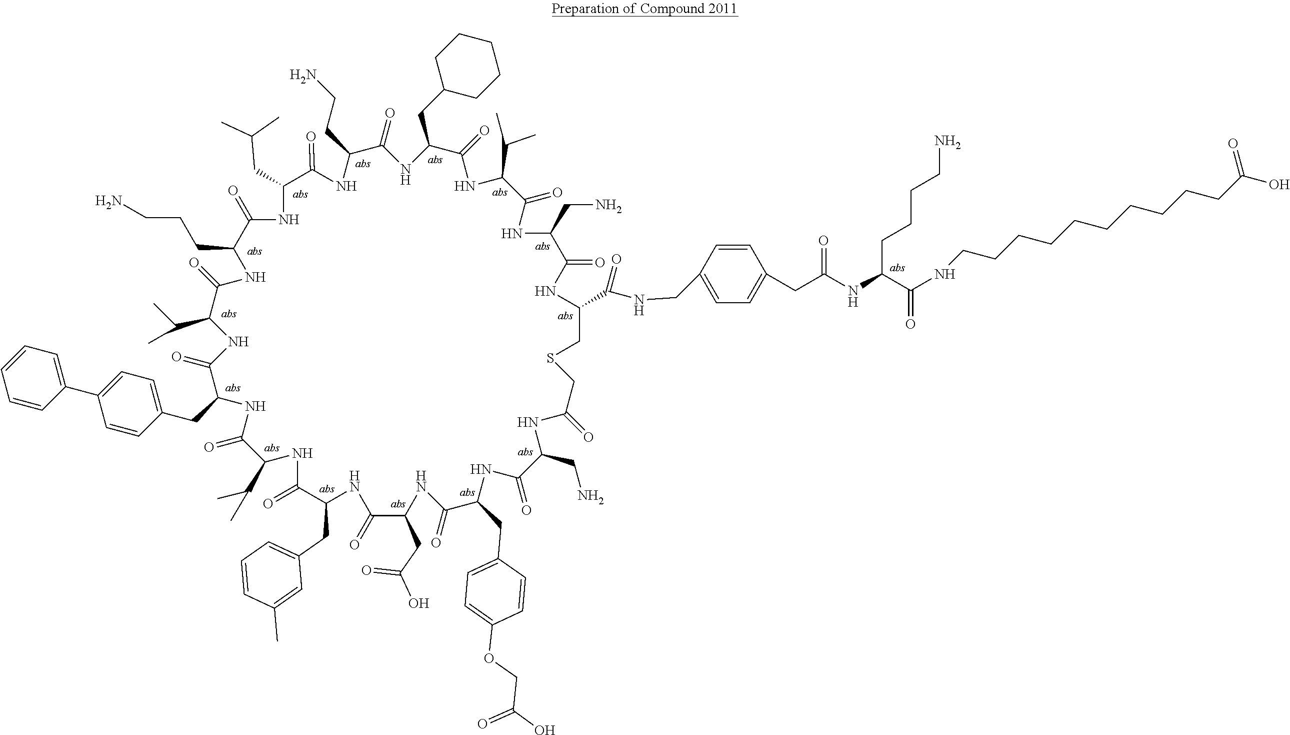 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCc3ccc(CC(=O)N[C@@H](CCCCN)C(=O)NCCCCCCCCCCC(=O)O)cc3)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2011 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 2-(4-(((((9H-fluoren-9-yl) methoxy) carbonyl)amino)methyl)phenyl) acetic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.6 mg, and its estimated purity by LCMS analysis was 96.4%. Analysis condition 1: Retention time=1.53 min; ESI-MS(+) m/z [M+2H]²⁺: 1146.

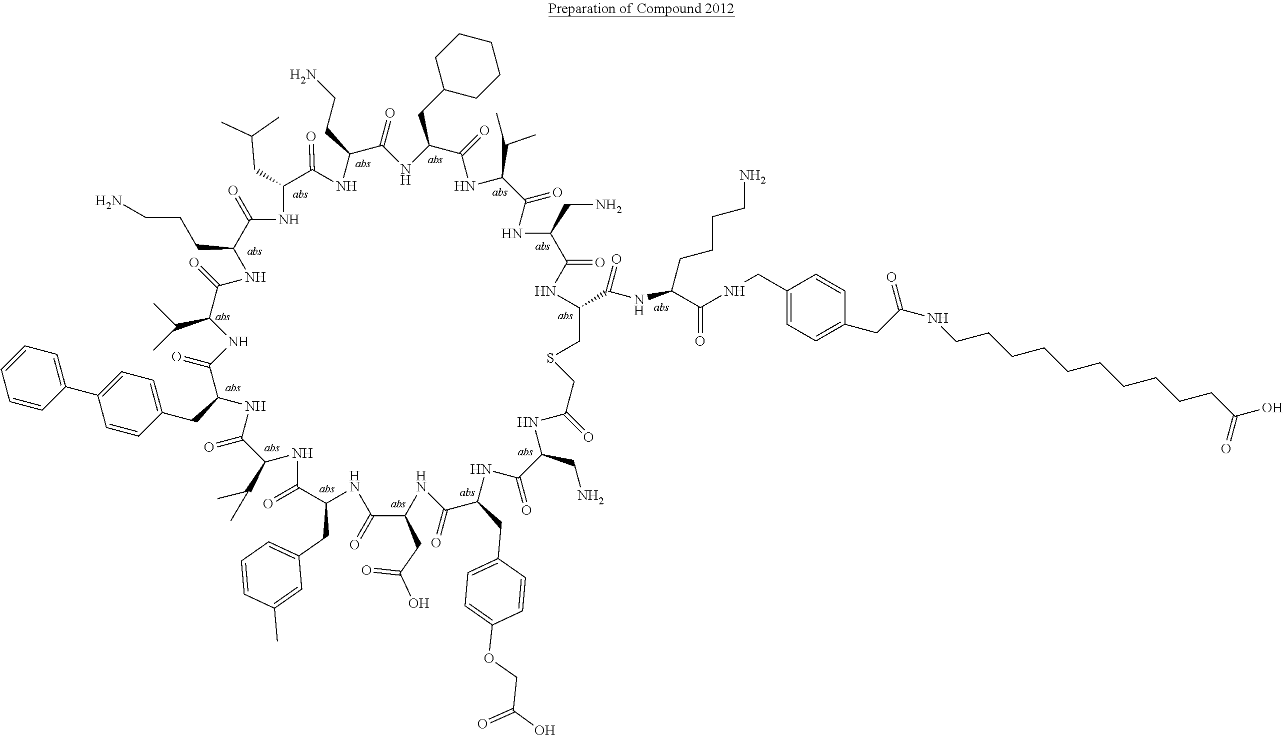 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCc3ccc(CC(=O)NCCCCCCCCCCC(=O)O)cc3)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2012 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 2-(4-(((((9H-fluoren-9-yl) methoxy) carbonyl)amino)methyl)phenyl) acetic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 23.3 mg, and its estimated purity by LCMS analysis was 100%. Analysis condition 2: Retention time=1.63 min; ESI-MS(+) m/z [M+2H]²⁺: 1146.1.

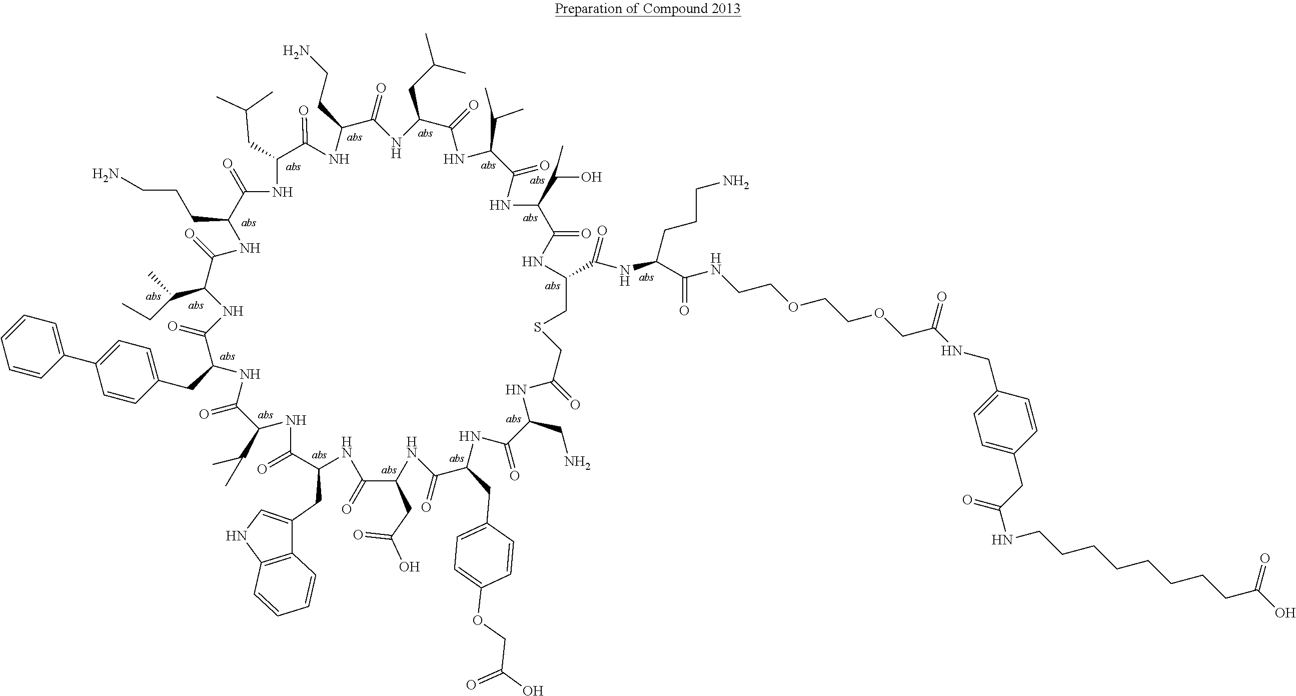 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCOCCOCC(=O)NCc2ccc(CC(=O)NCCCCCCCCC(=O)O)cc2)NC(=O)[C@H](C(C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2013 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 2-(4-(((((9H-fluoren-9-yl) methoxy) carbonyl)amino)methyl)phenyl) acetic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.2 mg, and its estimated purity by LCMS analysis was 90.6%. Analysis condition 2: Retention time=1.61 min; ESI-MS(+) m/z [M+2H]²⁺: 1250.

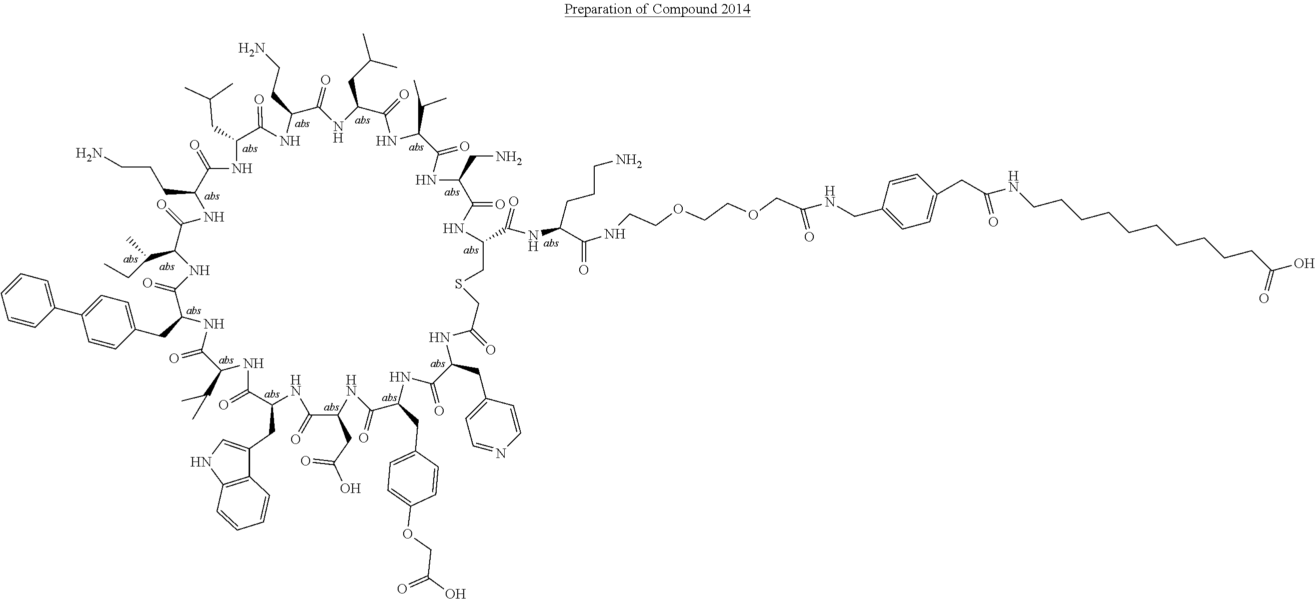 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCCN)C(=O)NCCOCCOCC(=O)NCc2ccc(CC(=O)NCCCCCCCCCCC(=O)O)cc2)NC(=O)[C@H](CN)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2014 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 2-(4-(((((9H-fluoren-9-yl) methoxy) carbonyl)amino)methyl)phenyl) acetic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 10.2 mg, and its estimated purity by LCMS analysis was 92.1%. Analysis condition 2: Retention time=1.44 min; ESI-MS(+) m/z [M+3H]³⁺: 828.6.

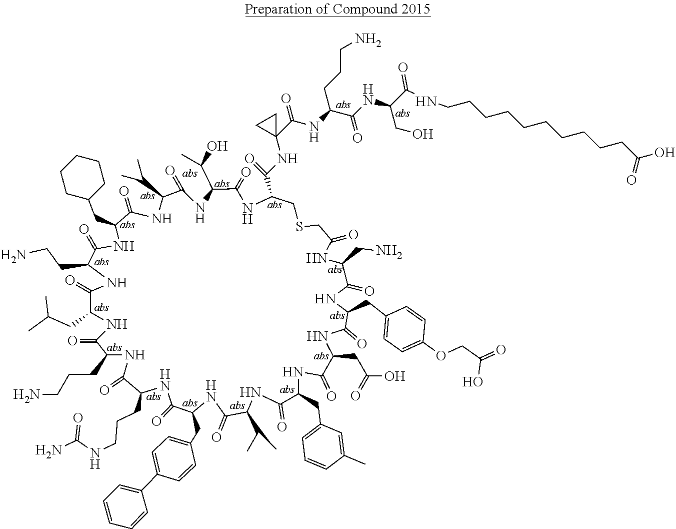 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NC3(C(=O)N[C@@H](CCCN)C(=O)N[C@H](CO)C(=O)NCCCCCCCCCCC(=O)O)CC3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2015 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.3 mg, and its estimated purity by LCMS analysis was 91.7%. Analysis condition 1: Retention time=1.6 min; ESI-MS(+) m/z [M+2H]²⁺: 1186.9.

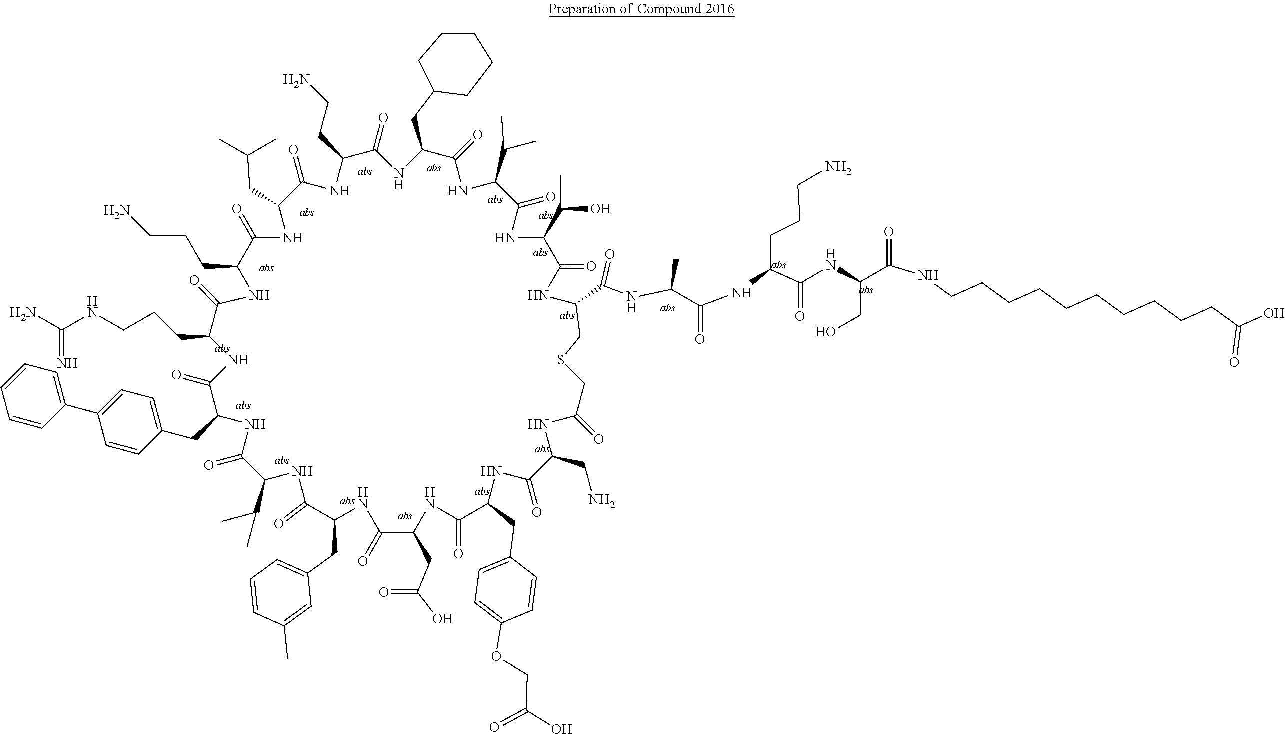 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCCN)C(=O)N[C@H](CO)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 2016 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.4 mg, and its estimated purity by LCMS analysis was 91.8%. Analysis condition 1: Retention time=1.6 min; ESI-MS(+) m/z [M+2H]²⁺: 1180.3.

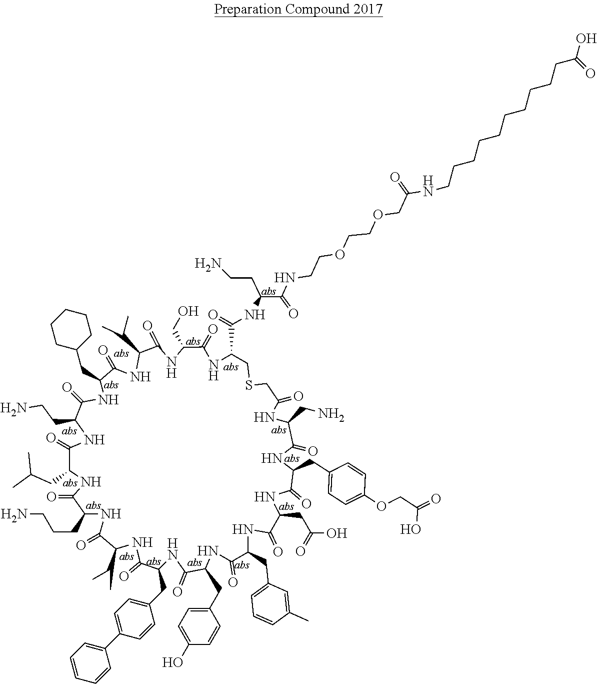 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](Cc3ccc(O)cc3)NC2=O)c1]

Compound 2017 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 26.9 mg, and its estimated purity by LCMS analysis was 94.2%. Analysis condition 1: Retention time=1.58 min; ESI-MS(+) m/z [M+2H]²⁺: 1163.3.

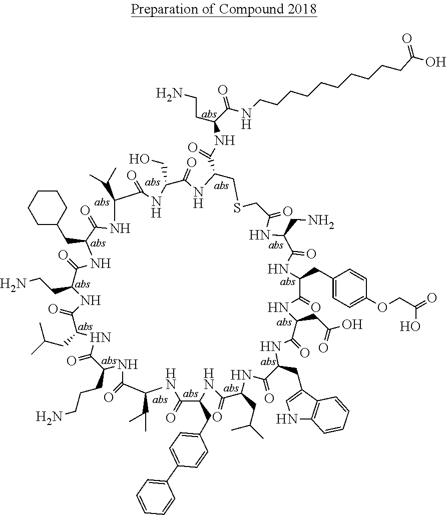 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 2018 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.3 mg, and its estimated purity by LCMS analysis was 90.8%. Analysis condition 2: Retention time=1.77 min; ESI-MS(+) m/z [M+3H]³⁺: 719.1.

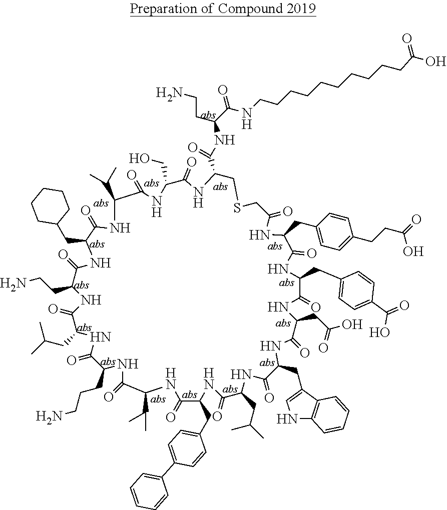 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(CCC(=O)O)cc2)NC(=O)CSC[C@@H](C(=O)N[C@@H](CCN)C(=O)NCCCCCCCCCCC(=O)O)NC(=O)[C@@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 2019 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 14.4 mg, and its estimated purity by LCMS analysis was 93.7%. Analysis condition 2: Retention time=1.68 min; ESI-MS(+) m/z [M+2H]²⁺: 1131.

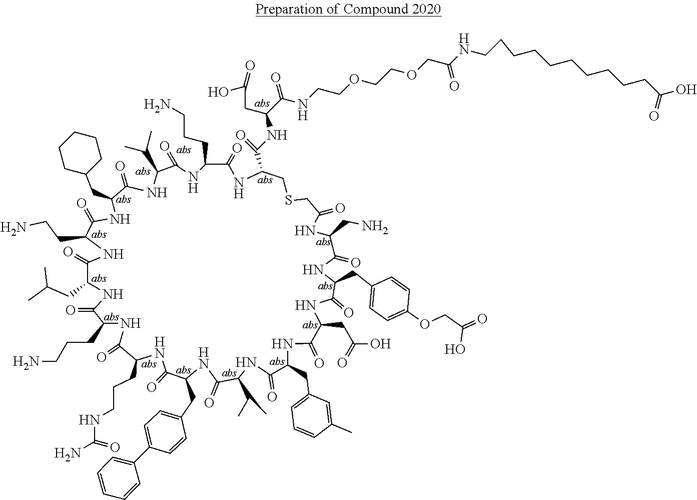 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)O)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2020 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 13.2 mg, and its estimated purity by LCMS analysis was 90.7%. Analysis condition 1: Retention time=1.56 min; ESI-MS(+) m/z [M+3H]³⁺: 787.6.

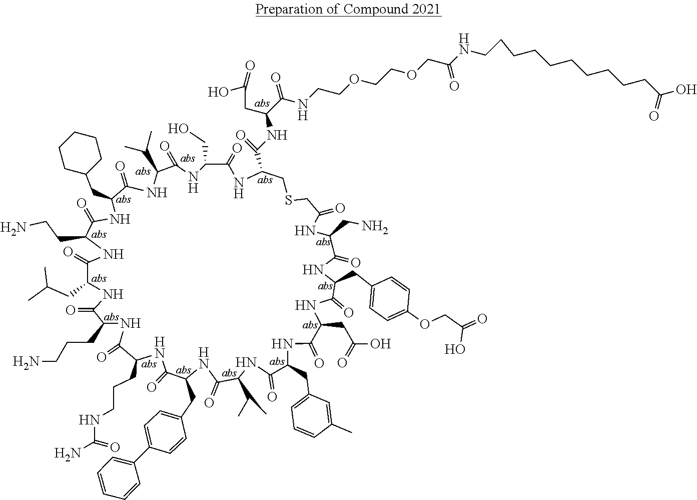 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CC(=O)O)C(=O)NCCOCCOCC(=O)NCCCCCCCCCCC(=O)O)NC(=O)[C@@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2021 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24 mg, and its estimated purity by LCMS analysis was 92.3%. Analysis condition 1: Retention time=1.66 min; ESI-MS(+) m/z [M+2H]²⁺: 1166.9.

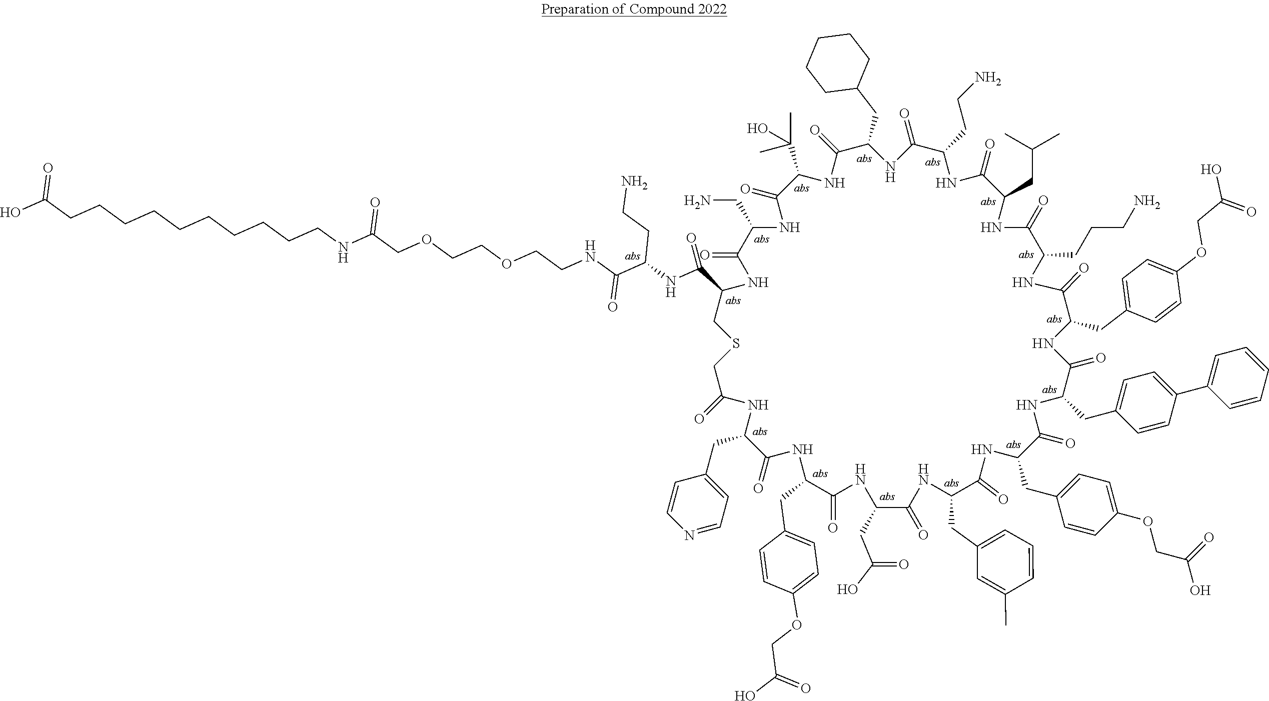 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2022 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.4 mg, and its estimated purity by LCMS analysis was 96.8%. Analysis condition: Retention time=1.52 min; ESI-MS(+) m/z [M+3H]³⁺: 862.2.

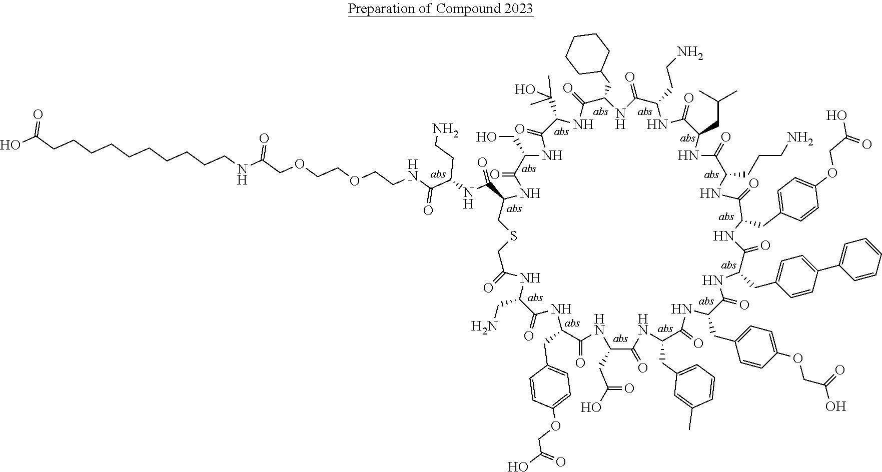 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2023 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-53% B over 28 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.8 mg, and its estimated purity by LCMS analysis was 100%. Analysis condition: Retention time=1.69 min; ESI-MS(+) m/z [M+2H]²⁺: 1261.4.

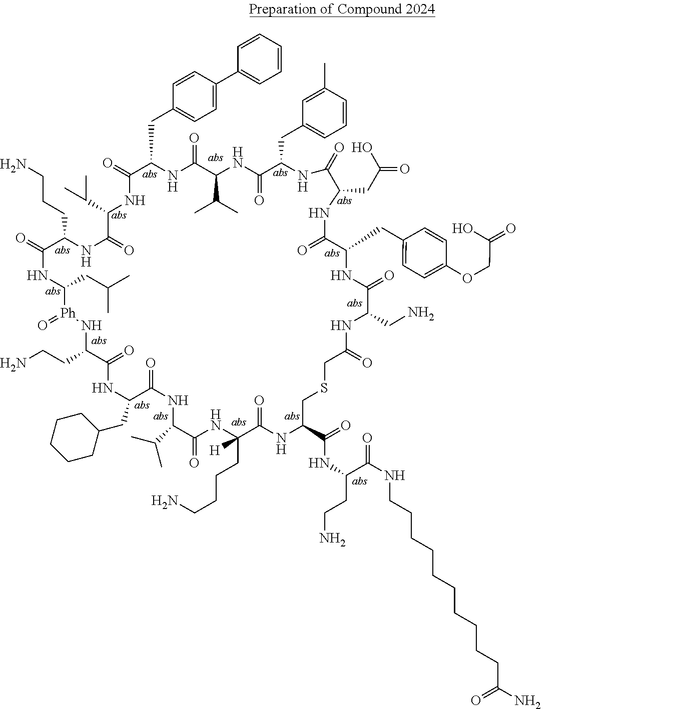

Compound 2024 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 9.7 mg, and its estimated purity by LCMS analysis was 96.3%. Analysis condition 1: Retention time=1.37 min; ESI-MS(+) m/z [M+3H]³⁺: 719.8.

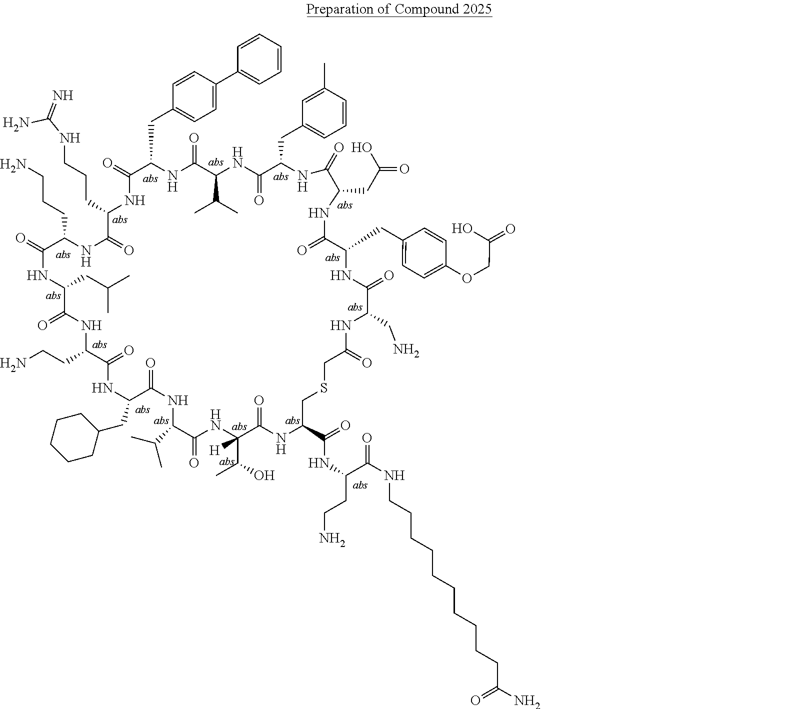 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 2025 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 45.1 mg, and its estimated purity by LCMS analysis was 100%. Analysis condition 2: Retention time=1.74 min; ESI-MS(+) m/z [M+3H]³⁺: 730.

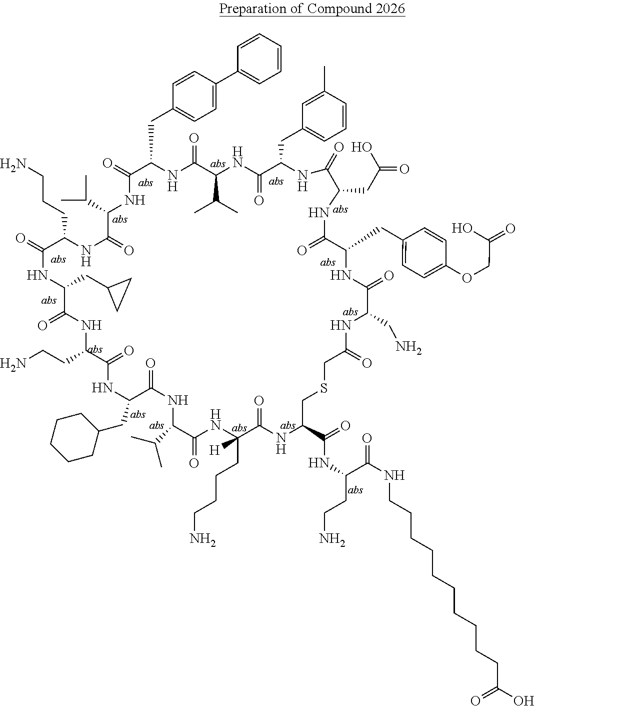 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC3CC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2026 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclopropyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.7 mg, and its estimated purity by LCMS analysis was 94%. Analysis condition 1: Retention time=1.36 min; ESI-MS(+) m/z [M+3H]³⁺: 719.2.

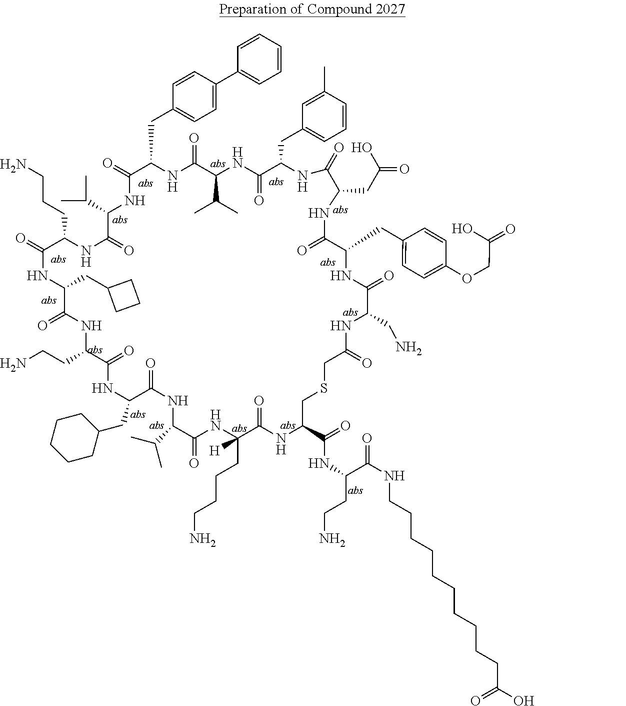 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC3CCC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2027 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclobutyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 26% B, 26-66% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 2.2 mg, and its estimated purity by LCMS analysis was 100%. Analysis condition 2: Retention time=1.63 min; ESI-MS(+) m/z [M+2H]²⁺: 1085.2.

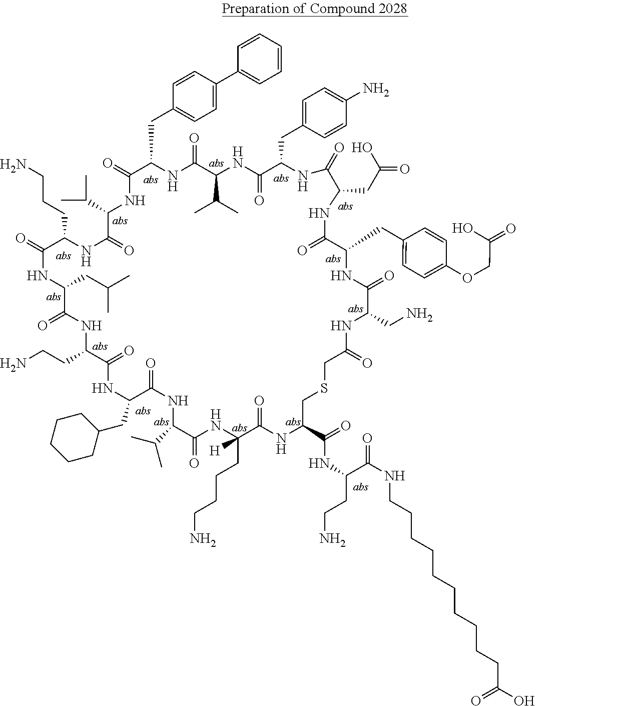 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccc(N)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2028 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-NHBoc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-45% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.9 mg, and its estimated purity by LCMS analysis was 93.2%. Analysis condition 2: Retention time=1.4 min; ESI-MS(+) m/z [M+2H]²⁺: 1080.1.

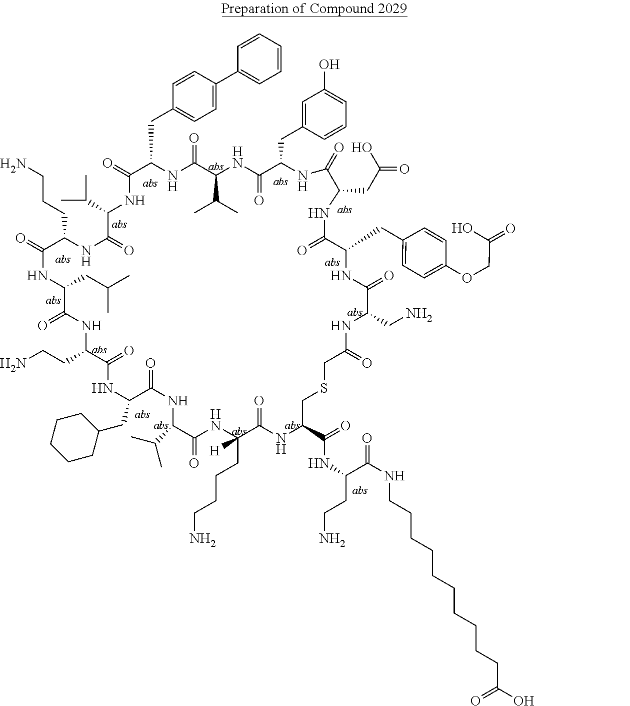 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(O)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2029 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-OtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-48% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.3 mg, and its estimated purity by LCMS analysis was 96%. Analysis condition 2: Retention time=1.35 min; ESI-MS(+) m/z [M+3H]³⁺: 720.8.

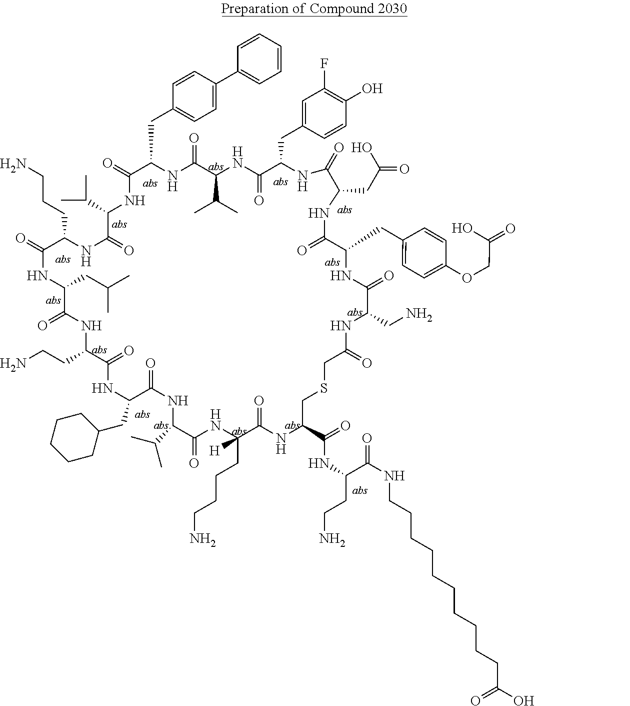 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccc(O)c(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2030 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 7.7 mg, and its estimated purity by LCMS analysis was 96.7%. Analysis condition 1: Retention time=1.52 min; ESI-MS(+) m/z [M+3H]³⁺: 726.5.

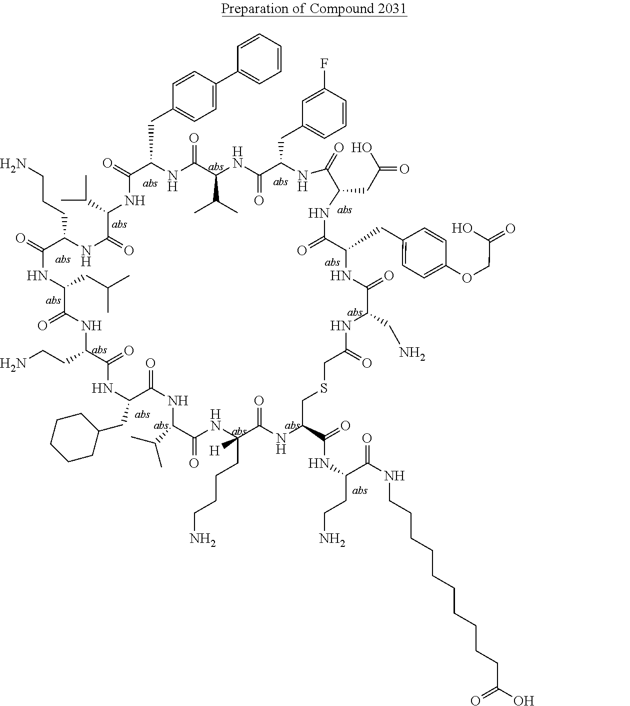 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2031 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3Cl)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.9 mg, and its estimated purity by LCMS analysis was 100%. Analysis condition 1: Retention time=1.43 min; ESI-MS(+) m/z [M+2H]²⁺: 1089.3.

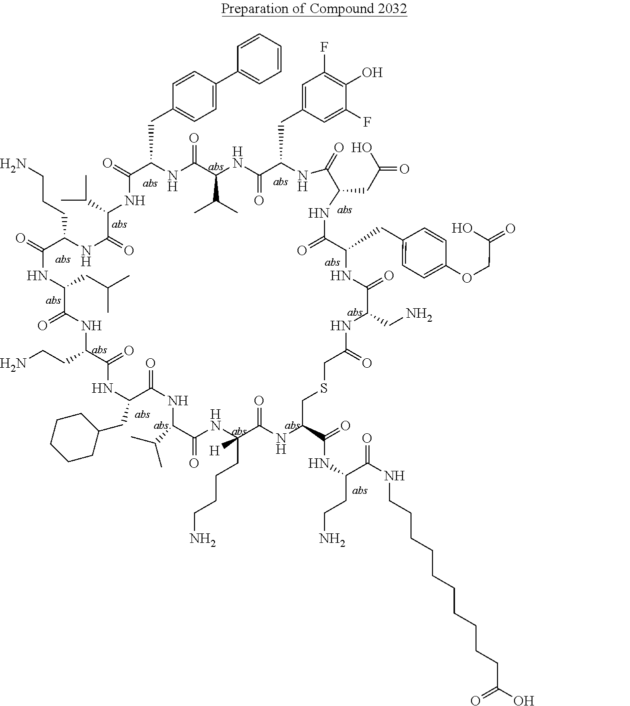 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cc(F)c(O)c(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2032 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(2,6-diF)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-50% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.4 mg, and its estimated purity by LCMS analysis was 100%. Analysis condition 2: Retention time=1.5 min; ESI-MS(+) m/z [M+2H]²⁺: 1098.8.

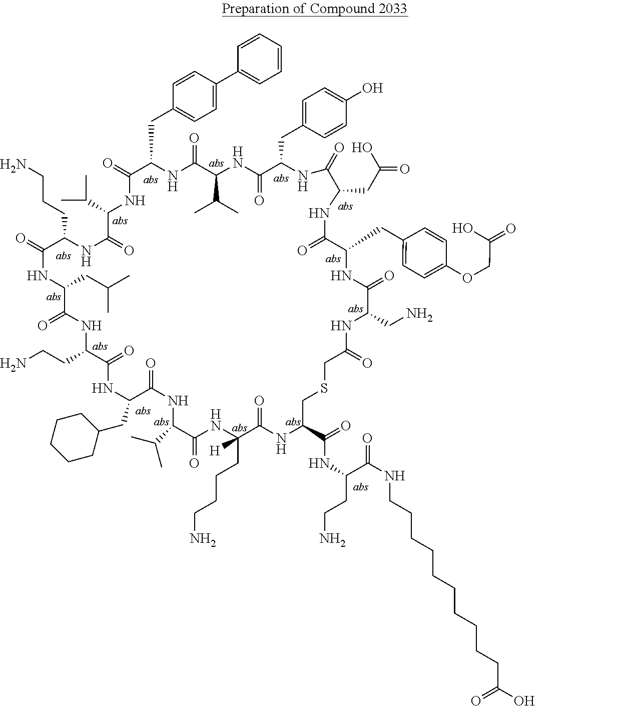 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccc(O)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2033 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-50% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.6 mg, and its estimated purity by LCMS analysis was 95.5%. Analysis condition 1: Retention time=1.29 min; ESI-MS(+) m/z [M+3H]³⁺: 721.1.

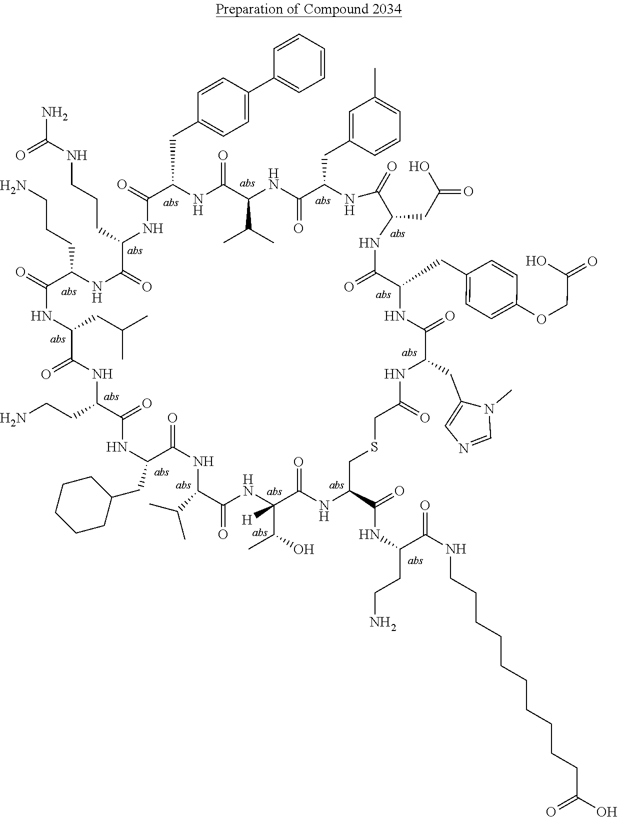 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cncn3C)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2034 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.4 mg, and its estimated purity by LCMS analysis was 99.3%. Analysis condition 1: Retention time=1.6 min; ESI-MS(+) m/z [M+3H]³⁺: 752.2.

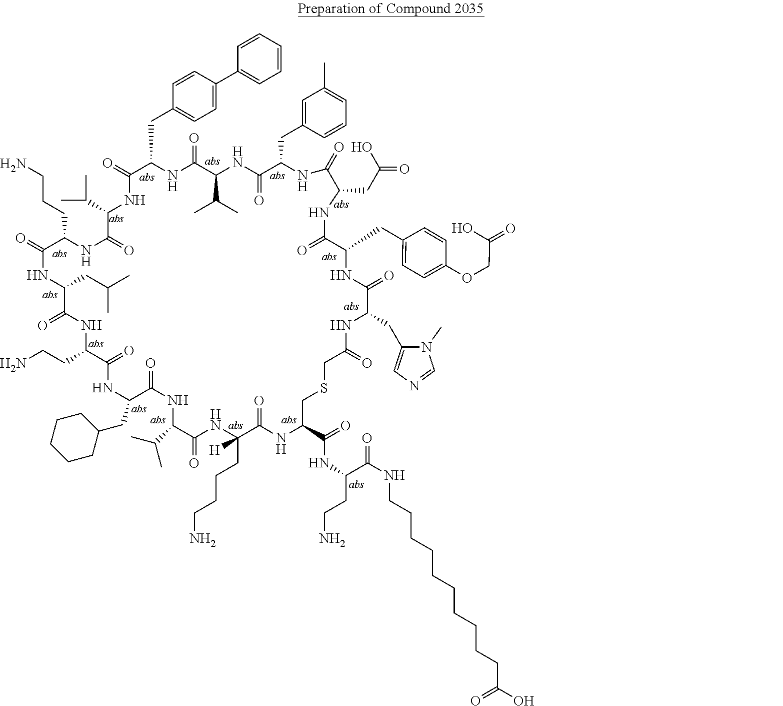 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cncn3C)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2035 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.3 mg, and its estimated purity by LCMS analysis was 100%. Analysis condition 1: Retention time=1.39 min; ESI-MS(+) m/z [M+2H]²⁺: 1112.1.

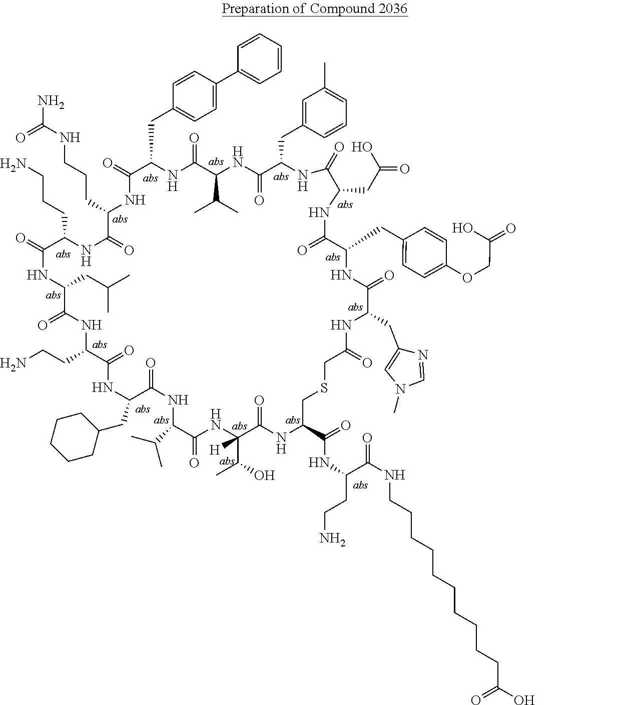 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cn(C)cn3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2036 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-53% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.1 mg, and its estimated purity by LCMS analysis was 93.4%. Analysis condition 1: Retention time=1.48 min; ESI-MS(+) m/z [M+3H]³⁺: 752.

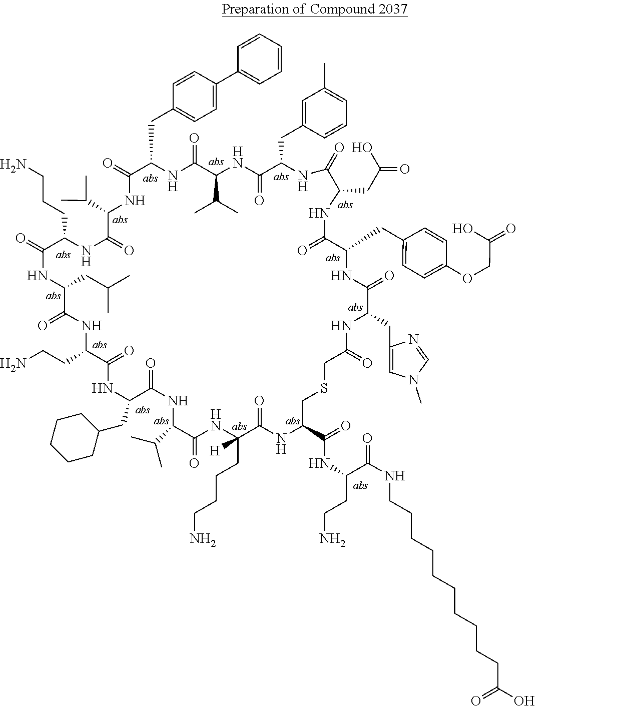 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cn(C)cn3)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2037 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-50% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.6 mg, and its estimated purity by LCMS analysis was 86.7%. Analysis condition 2: Retention time=1.44 min; ESI-MS(+) m/z [M+2H]²⁺: 1112.

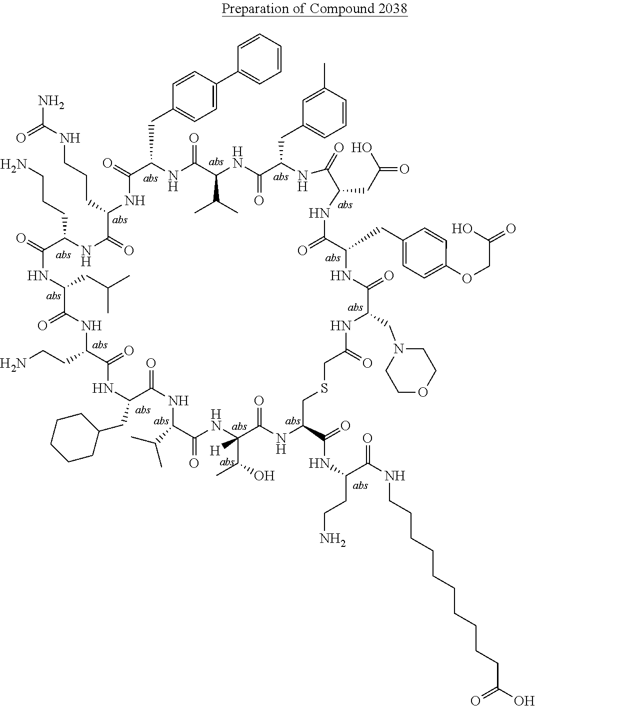 [SMILES: Cc1cccc(C[C@@H]2NC(=O)[C@H](CC(=O)O)NC(=O)[C@H](Cc3ccc(OCC(=O)O)cc3)NC(=O)[C@H](CN3CCOCC3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2038 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with(S)-2-((((9H-fluoren-9-yl) methoxy) carbonyl)amino)-3-morpholinopropanoic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 7.1 mg, and its estimated purity by LCMS analysis was 95.9% as a mixture of diastereomers. Analysis condition 2: Retention time=1.65, 1.68 min; ESI-MS(+) m/z [M+2H]²⁺: 1130.01, 1130.12.

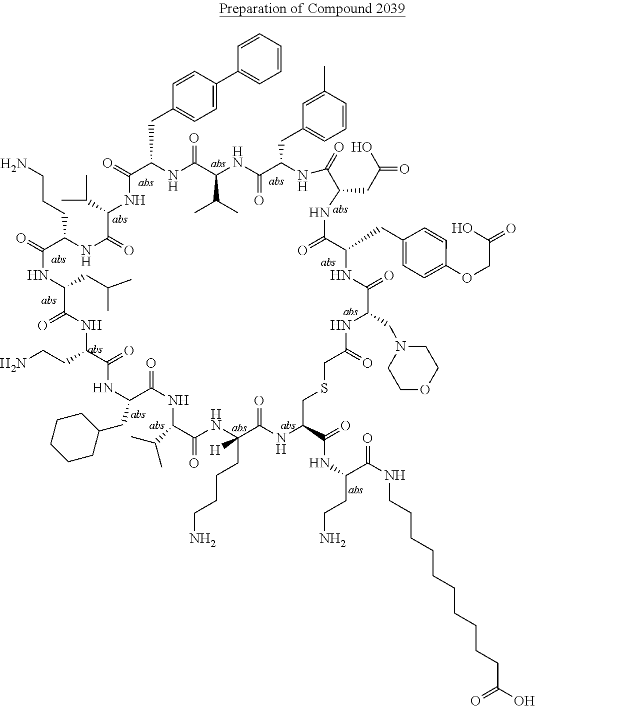 [SMILES: Cc1cccc(C[C@@H]2NC(=O)[C@H](CC(=O)O)NC(=O)[C@H](Cc3ccc(OCC(=O)O)cc3)NC(=O)[C@H](CN3CCOCC3)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2039 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with(S)-2-((((9H-fluoren-9-yl) methoxy) carbonyl)amino)-3-morpholinopropanoic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.6 mg, and its estimated purity by LCMS analysis was 88.7% as a mixture of diastereomers. Analysis condition 1: Retention time=1.41, 1.43 min; ESI-MS(+) m/z [M+2H]²⁺: 743.1.

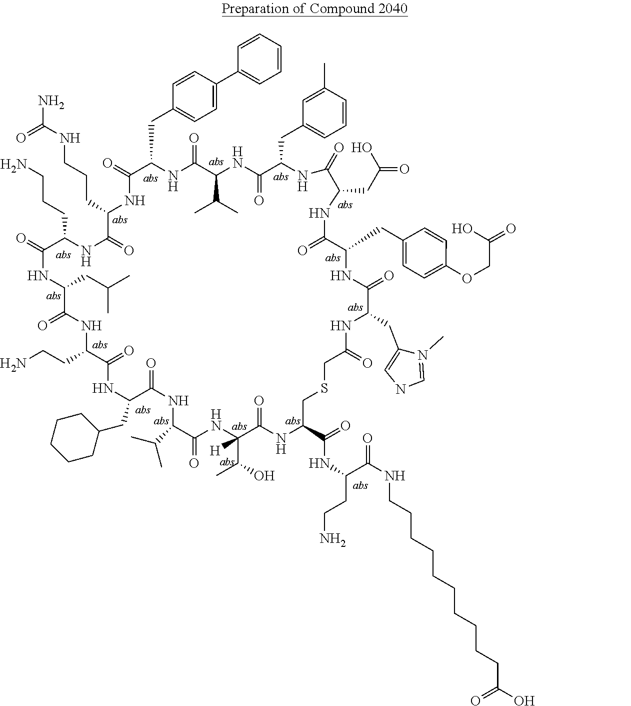

Compound 2040 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.3 mg, and its estimated purity by LCMS analysis was 94.6% as a mixture of diastereomers. Analysis condition 2: Retention time=1.64, 1.67 min; ESI-MS(+) m/z [M+3H]³⁺: 752.03, 752.03.

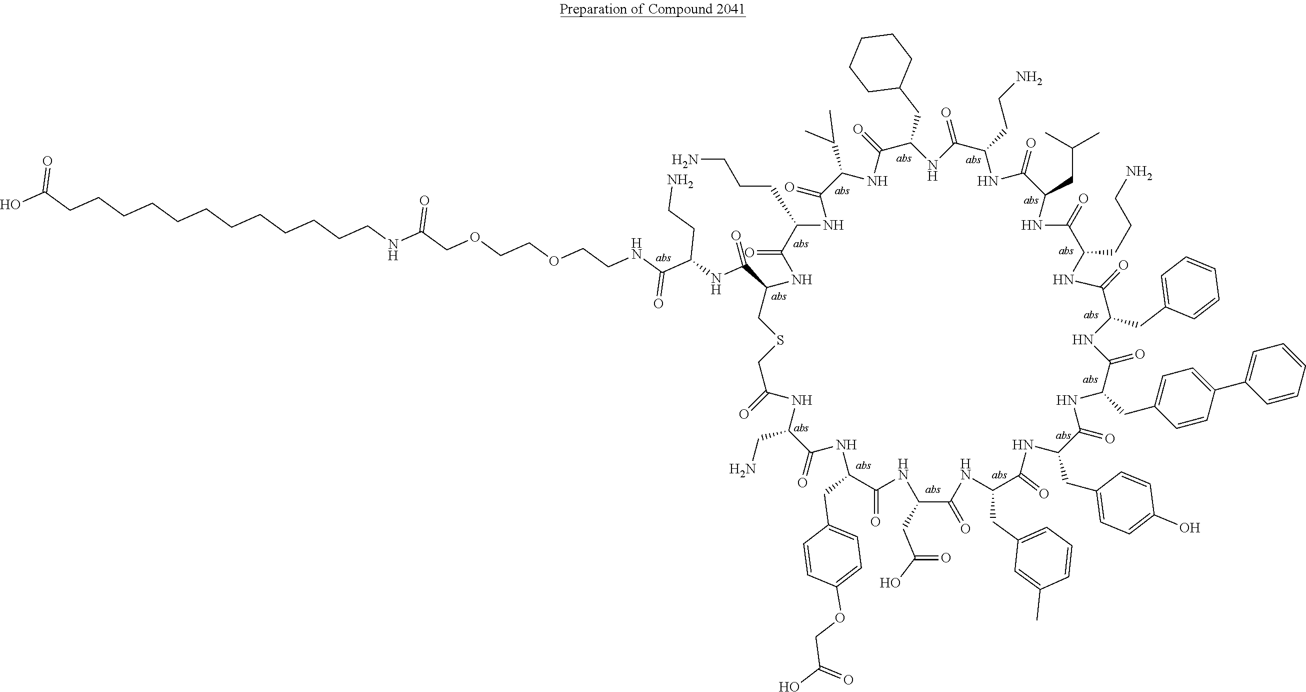 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccccc3)NC(=O)[C@H](Cc3ccc(-c4ccccc4)cc3)NC(=O)[C@H](Cc3ccc(O)cc3)NC2=O)c1]

Compound 2041 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 22.2 mg, and its estimated purity by LCMS analysis was 95.8%. Analysis condition 1: Retention time=1.57 min; ESI-MS(+) m/z [M+2H]²⁺: 1201.2.

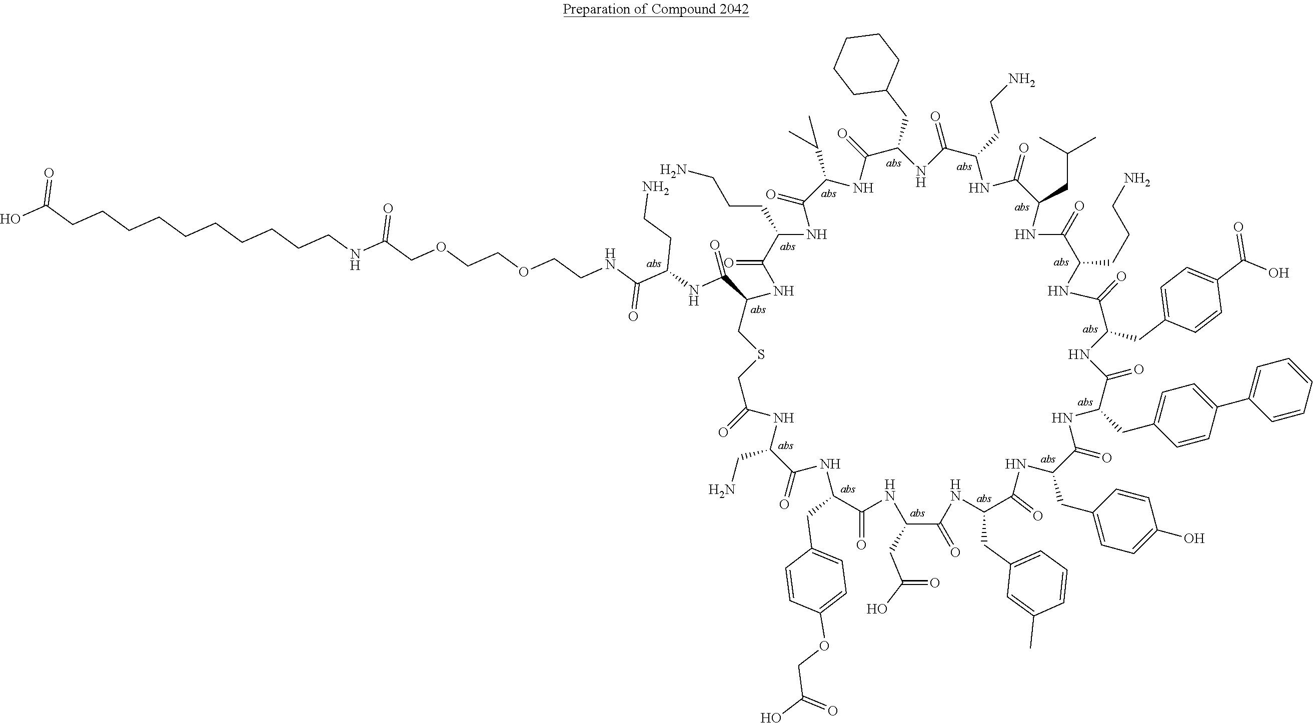 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(O)cc3)NC2=O)c1]

Compound 2042 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 40.7 mg, and its estimated purity by LCMS analysis was 96.1%. Analysis condition 1: Retention time=1.51 min; ESI-MS(+) m/z [M+2H]²⁺: 1222.9.

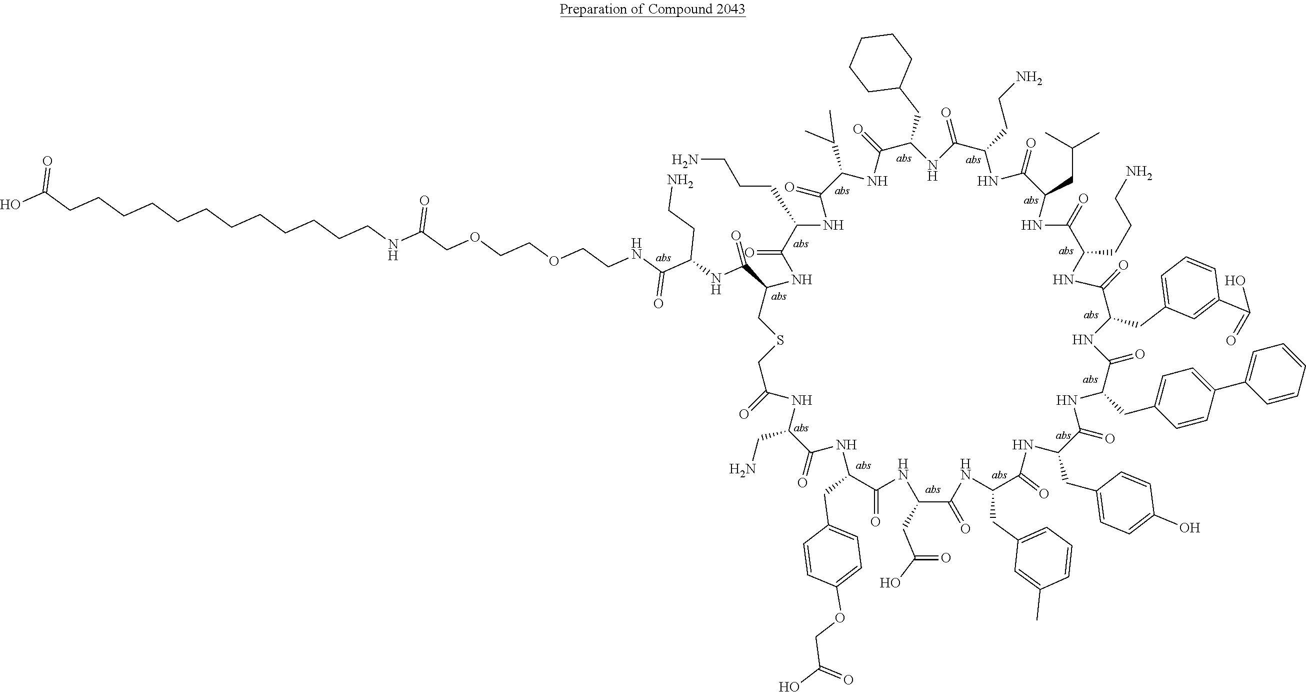 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3cccc(C(=O)O)c3)NC(=O)[C@H](Cc3ccc(-c4ccccc4)cc3)NC(=O)[C@H](Cc3ccc(O)cc3)NC2=O)c1]

Compound 2043 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 27 mg, and its estimated purity by LCMS analysis was 95.4%. Analysis condition 1: Retention time=1.57 min; ESI-MS(+) m/z [M+3H]³⁺: 816.

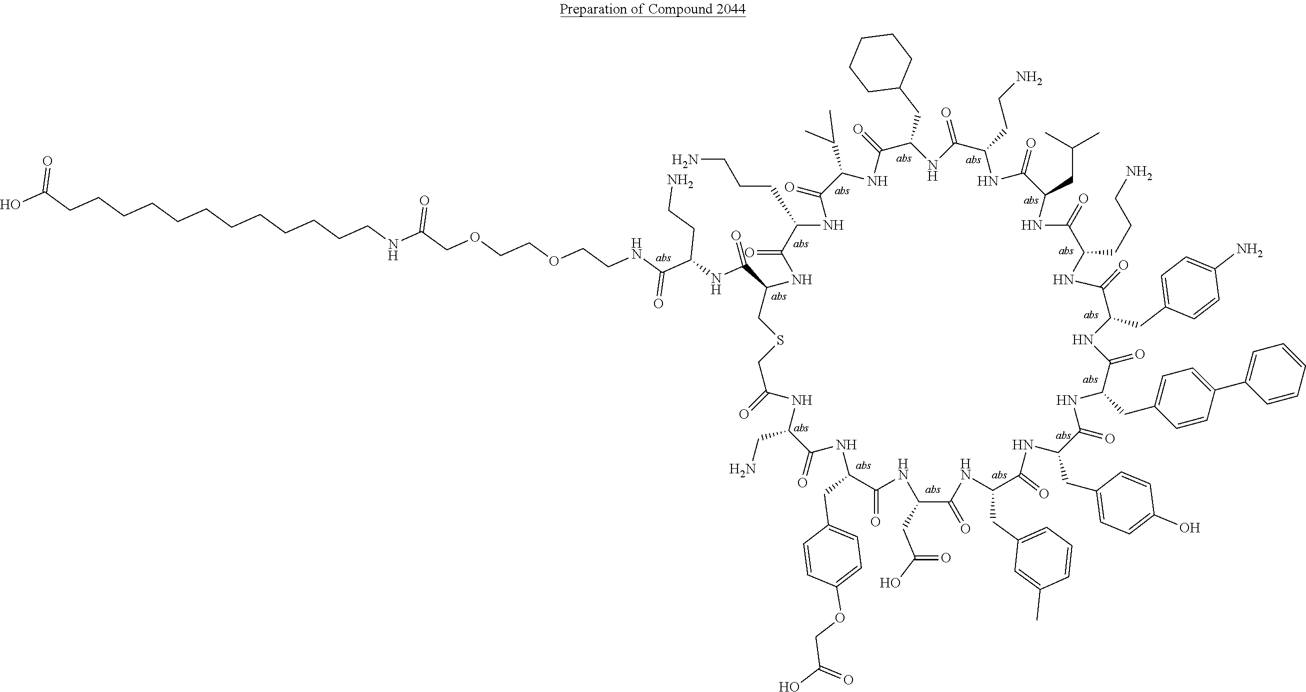 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(N)cc3)NC(=O)[C@H](Cc3ccc(-c4ccccc4)cc3)NC(=O)[C@H](Cc3ccc(O)cc3)NC2=O)c1]

Compound 2044 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-NHBoc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 57.1 mg, and its estimated purity by LCMS analysis was 91.6%. Analysis condition 2: Retention time=1.57 min; ESI-MS(+) m/z [M+3H]³⁺: 806.1.

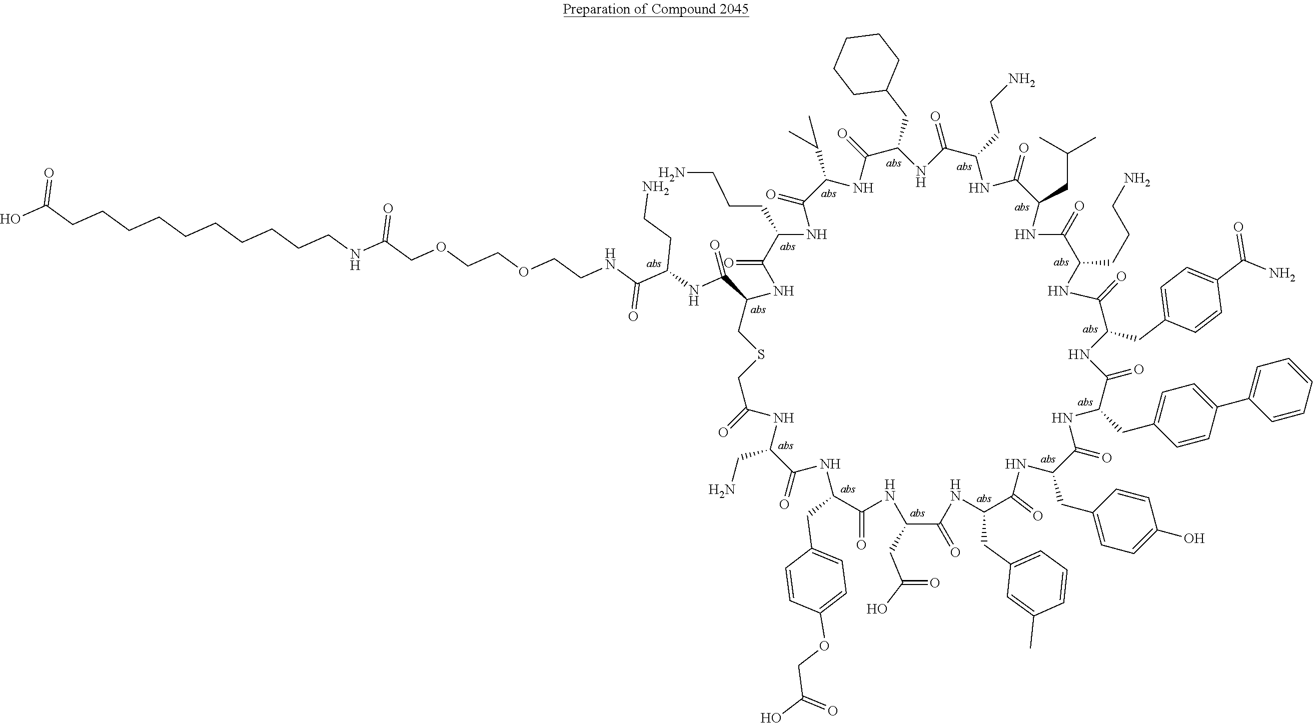 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(N)=O)cc3)NC(=O)[C@H](Cc3ccc(-c4ccccc4)cc3)NC(=O)[C@H](Cc3ccc(O)cc3)NC2=O)c1]

Compound 2045 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-CONH₂)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 31 mg, and its estimated purity by LCMS analysis was 95%. Analysis condition 1: Retention time=1.55 min; ESI-MS(+) m/z [M+3H]³⁺: 815.1.

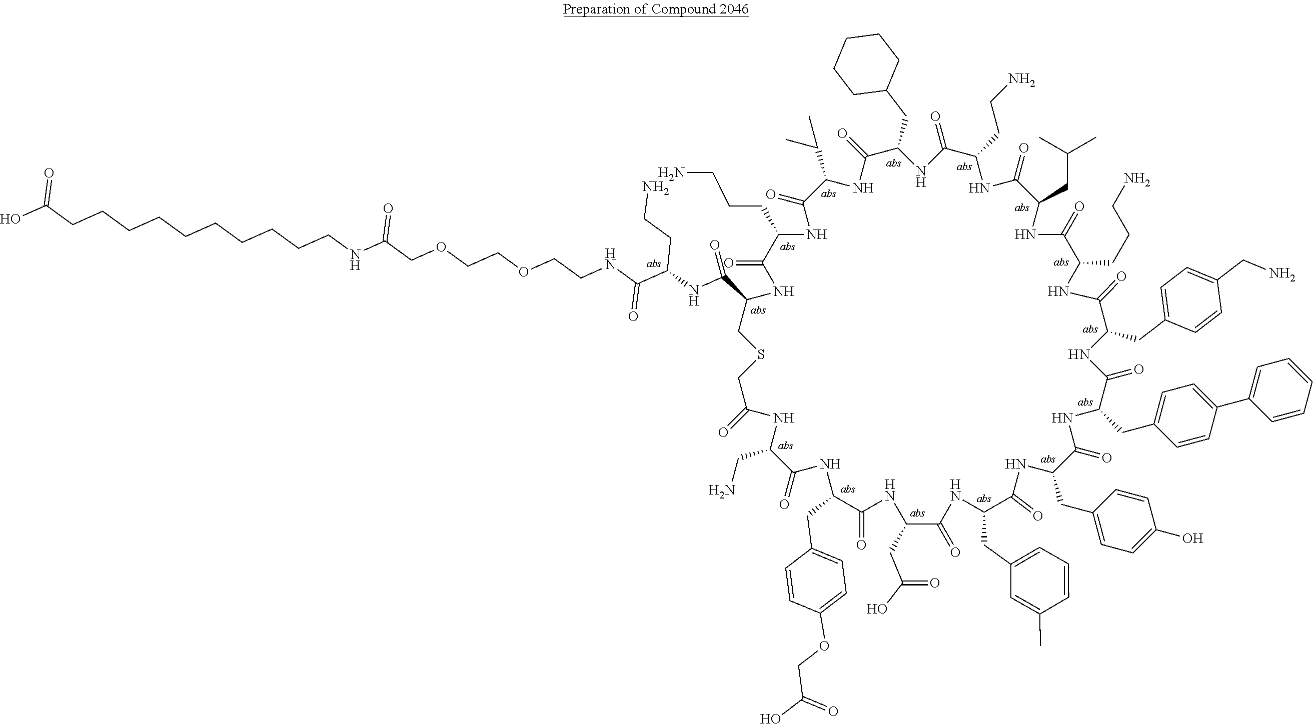 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(CN)cc3)NC(=O)[C@H](Cc3ccc(-c4ccccc4)cc3)NC(=O)[C@H](Cc3ccc(O)cc3)NC2=O)c1]

Compound 2046 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-CH₂NHBoc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 45.4 mg, and its estimated purity by LCMS analysis was 97.4%. Analysis condition 2: Retention time=1.46 min; ESI-MS(+) m/z [M+3H]³⁺: 811.

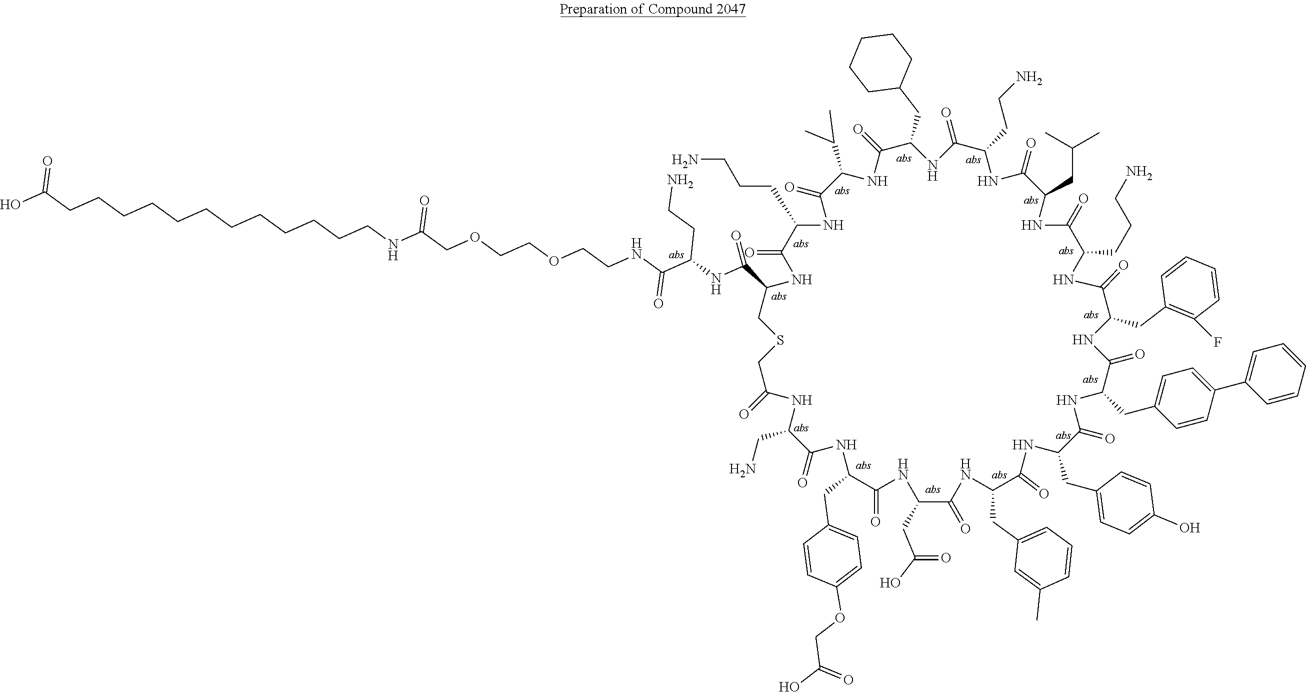 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccccc3F)NC(=O)[C@H](Cc3ccc(-c4ccccc4)cc3)NC(=O)[C@H](Cc3ccc(O)cc3)NC2=O)c1]

Compound 2047 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (2-F)—OH; “Symphony X Chloroacetic Anhydride coupling procedure”, “Symphony X Final rinse and dry procedure”, “Global Deprotection Method A”, “Cyclization Method A”.

, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 28 mg, and its estimated purity by LCMS analysis was 89.2%. Analysis condition 1: Retention time=1.61 min; ESI-MS(+) m/z [M+3H]³⁺: 807.3.

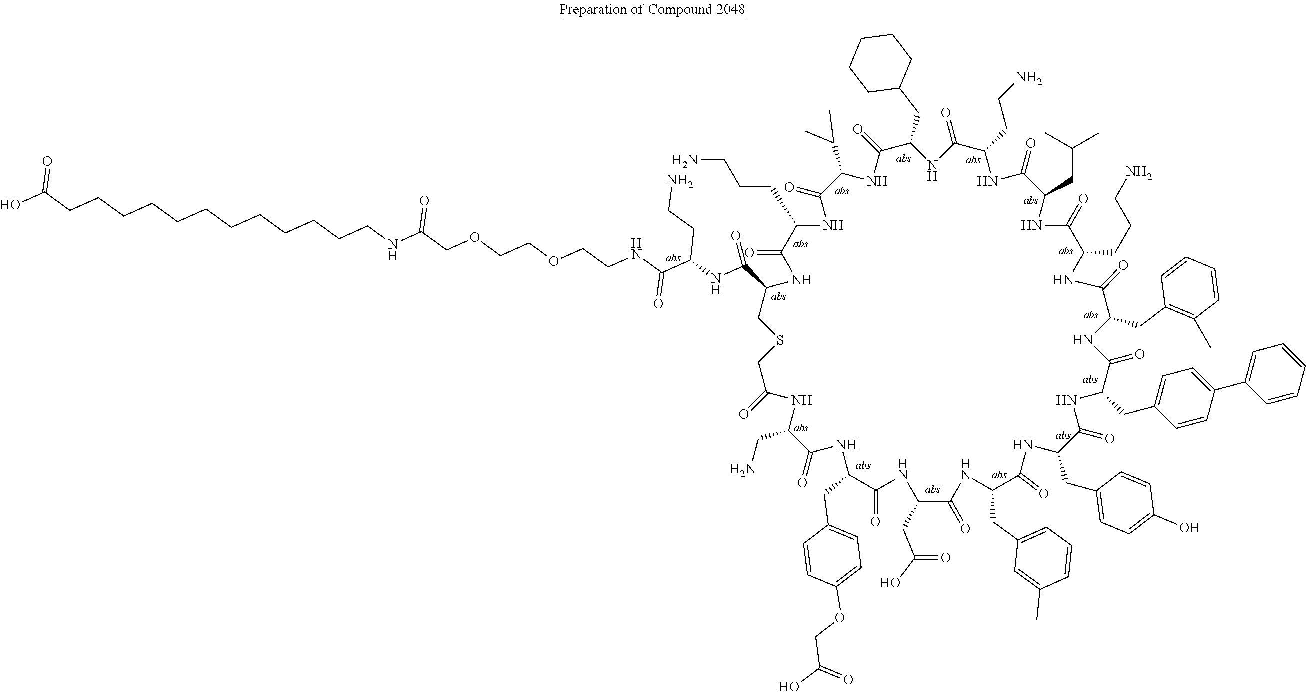 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccccc3C)NC(=O)[C@H](Cc3ccc(-c4ccccc4)cc3)NC(=O)[C@H](Cc3ccc(O)cc3)NC2=O)c1]

Compound 2048 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (2-Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 25% B, 25-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 20.3 mg, and its estimated purity by LCMS analysis was 99.1%. Analysis condition 2: Retention time=1.71 min; ESI-MS(+) m/z [M+2H]²⁺: 1208.3.

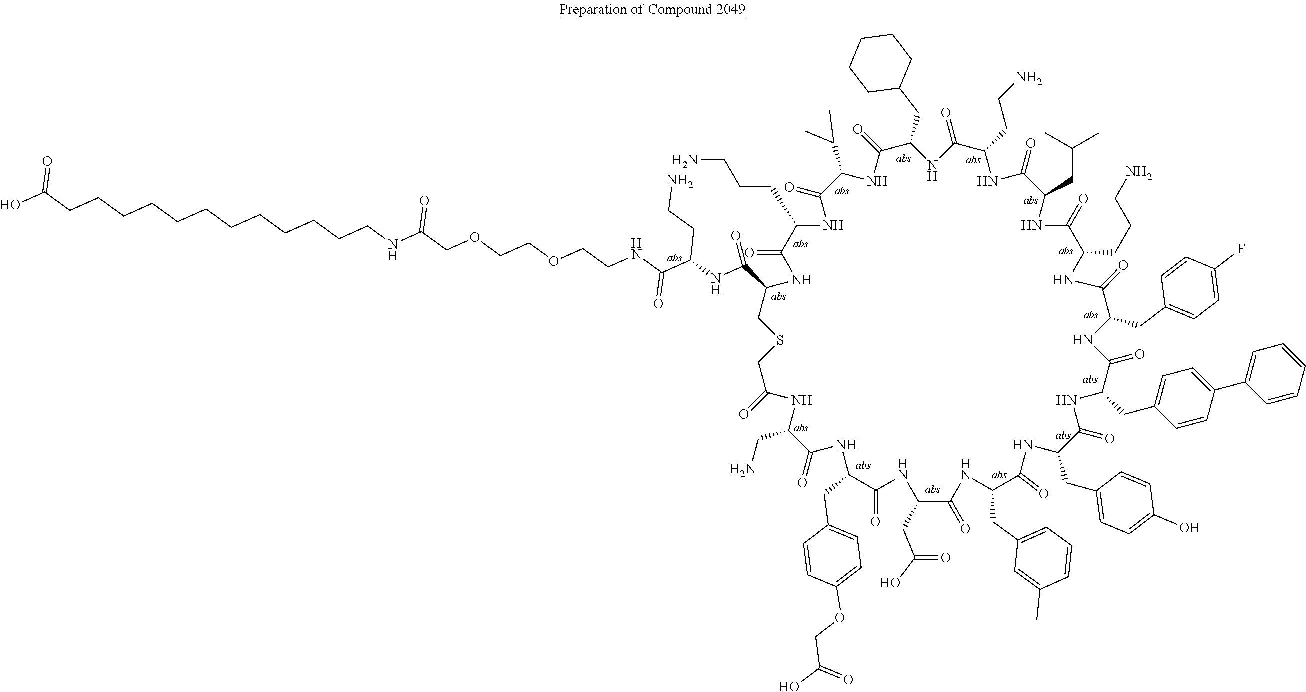 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(F)cc3)NC(=O)[C@H](Cc3ccc(-c4ccccc4)cc3)NC(=O)[C@H](Cc3ccc(O)cc3)NC2=O)c1]

Compound 2049 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 48.2 mg, and its estimated purity by LCMS analysis was 96%. Analysis condition 2: Retention time=1.61 min; ESI-MS(+) m/z [M+3H]³⁺: 807.

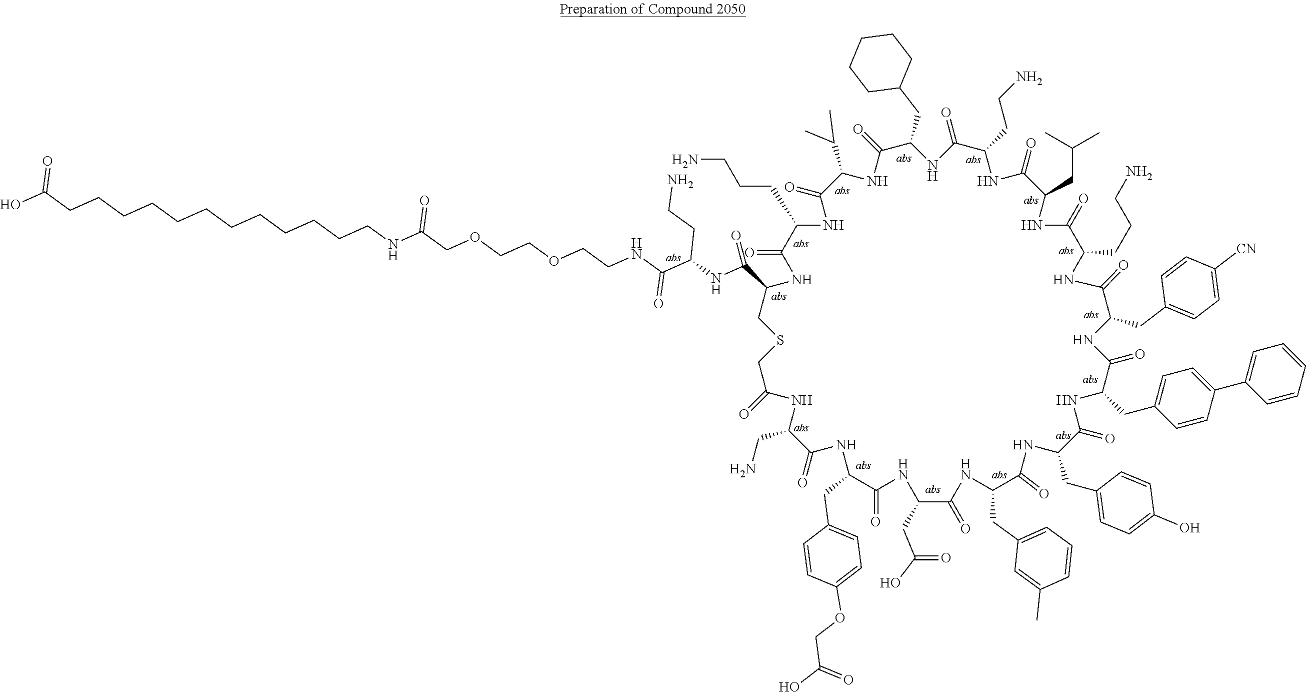 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C#N)cc3)NC(=O)[C@H](Cc3ccc(-c4ccccc4)cc3)NC(=O)[C@H](Cc3ccc(O)cc3)NC2=O)c1]

Compound 2050 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-CN)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 28 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 30 mg, and its estimated purity by LCMS analysis was 93.8%. Analysis condition 2: Retention time=1.62 min; ESI-MS(+) m/z [M+3H]³⁺: 809.1.

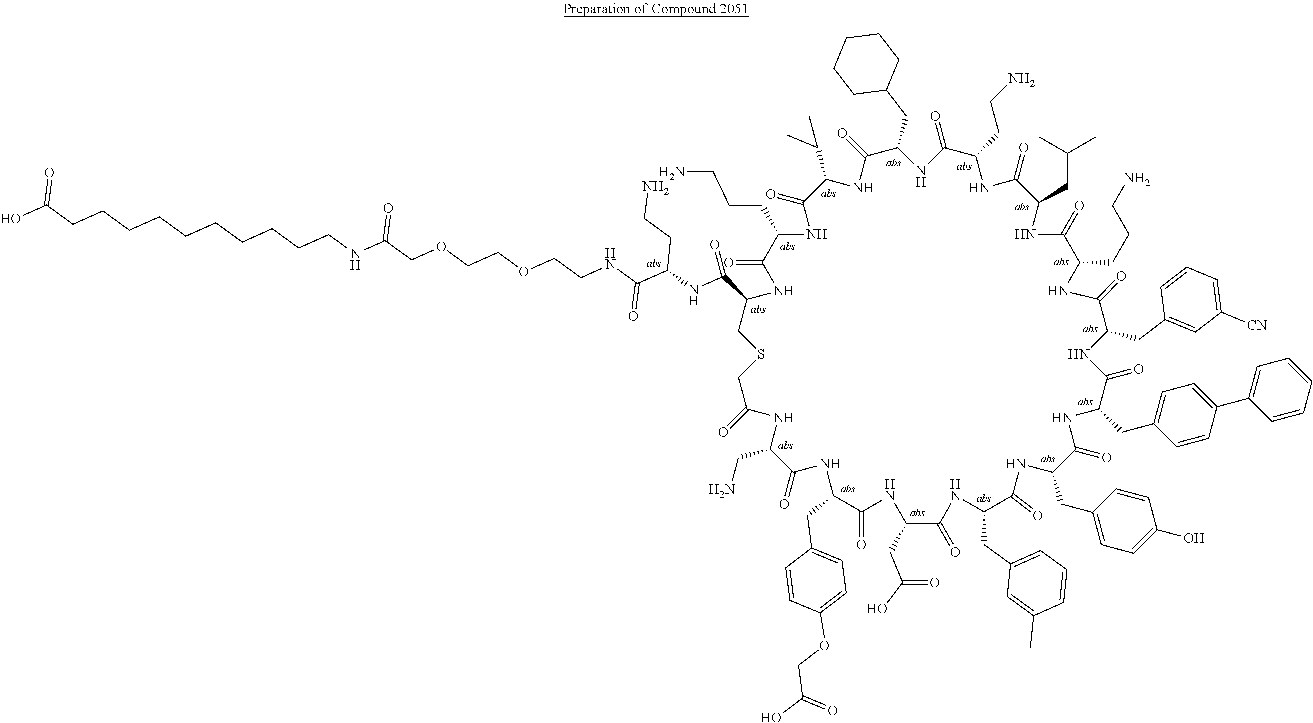 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3cccc(C#N)c3)NC(=O)[C@H](Cc3ccc(-c4ccccc4)cc3)NC(=O)[C@H](Cc3ccc(O)cc3)NC2=O)c1]

Compound 2051 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phen (3-CN)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 57.9 mg, and its estimated purity by LCMS analysis was 100%. Analysis condition 1: Retention time=1.5 min; ESI-MS(+) m/z [M+3H]³⁺: 809.1.

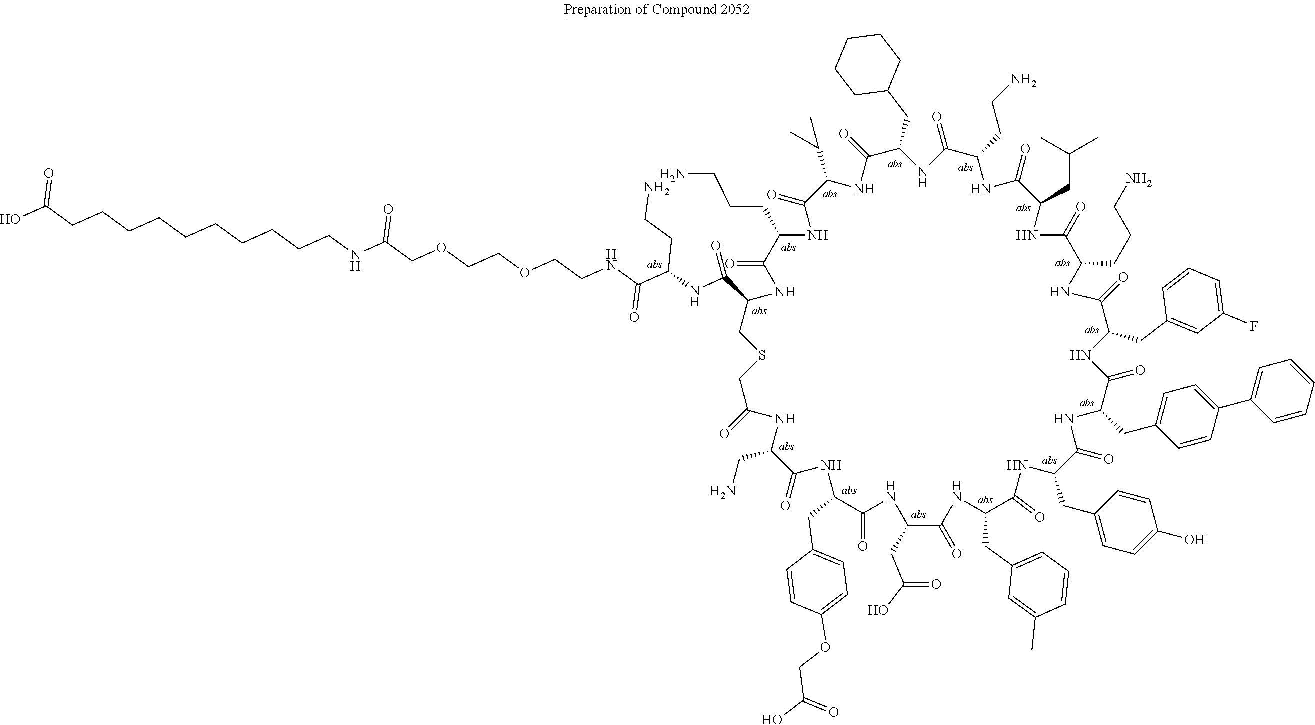 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3cccc(F)c3)NC(=O)[C@H](Cc3ccc(-c4ccccc4)cc3)NC(=O)[C@H](Cc3ccc(O)cc3)NC2=O)c1]

Compound 2052 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.5 mg, and its estimated purity by LCMS analysis was 99.4%. Analysis condition 2: Retention time=1.72 min; ESI-MS(+) m/z [M+2H]²⁺: 1210.

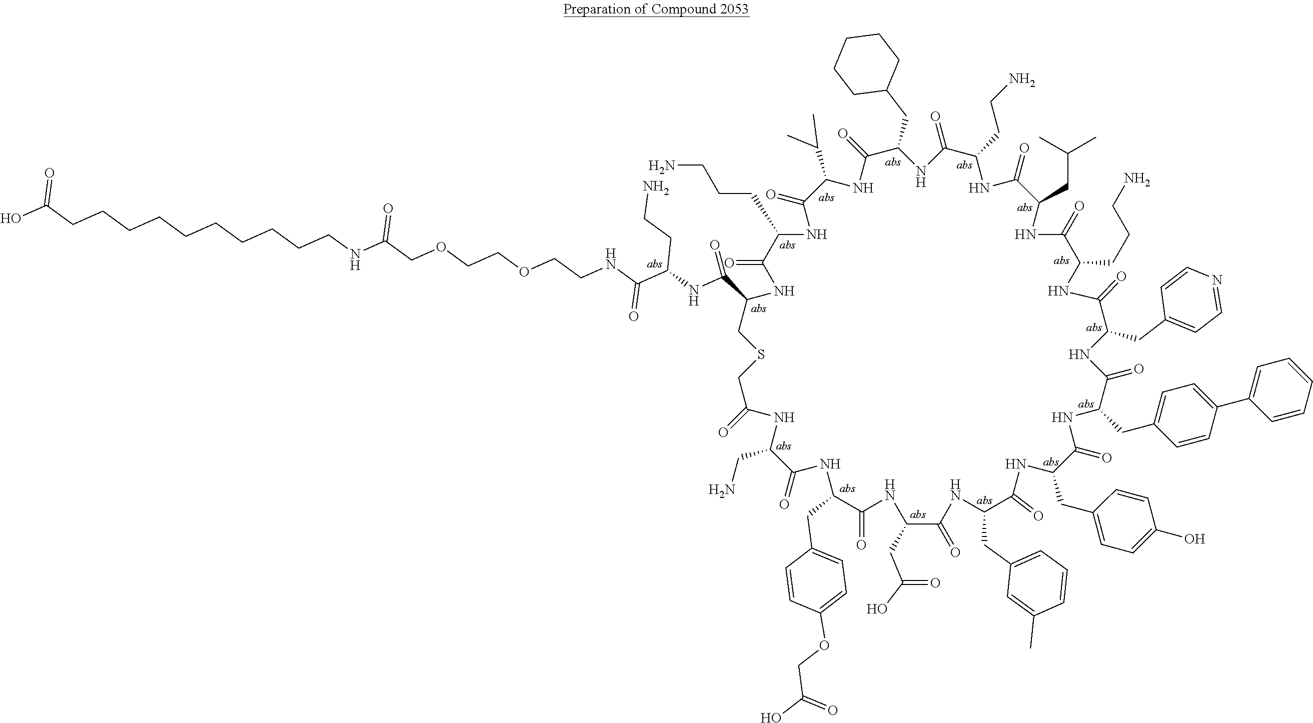 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccncc3)NC(=O)[C@H](Cc3ccc(-c4ccccc4)cc3)NC(=O)[C@H](Cc3ccc(O)cc3)NC2=O)c1]

Compound 2053 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Ala(4-pyrdyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 28.6 mg, and its estimated purity by LCMS analysis was 94.8%. Analysis condition 1: Retention time=1.47 min; ESI-MS(+) m/z [M+3H]³⁺: 801.

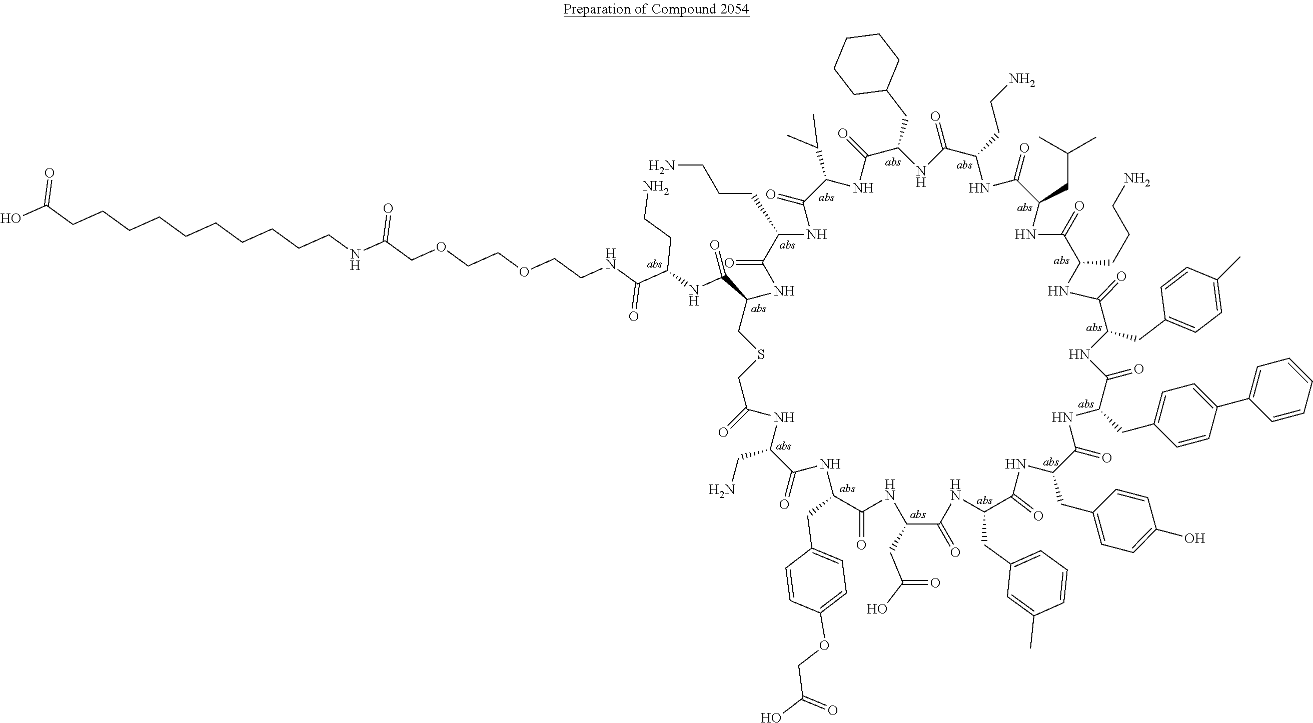 [SMILES: Cc1ccc(C[C@@H]2NC(=O)[C@H](Cc3ccc(-c4ccccc4)cc3)NC(=O)[C@H](Cc3ccc(O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 2054 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 25.4 mg, and its estimated purity by LCMS analysis was 85.6%. Analysis condition 2: Retention time=1.76 min; ESI-MS(+) m/z [M+2H]²⁺: 1207.9.

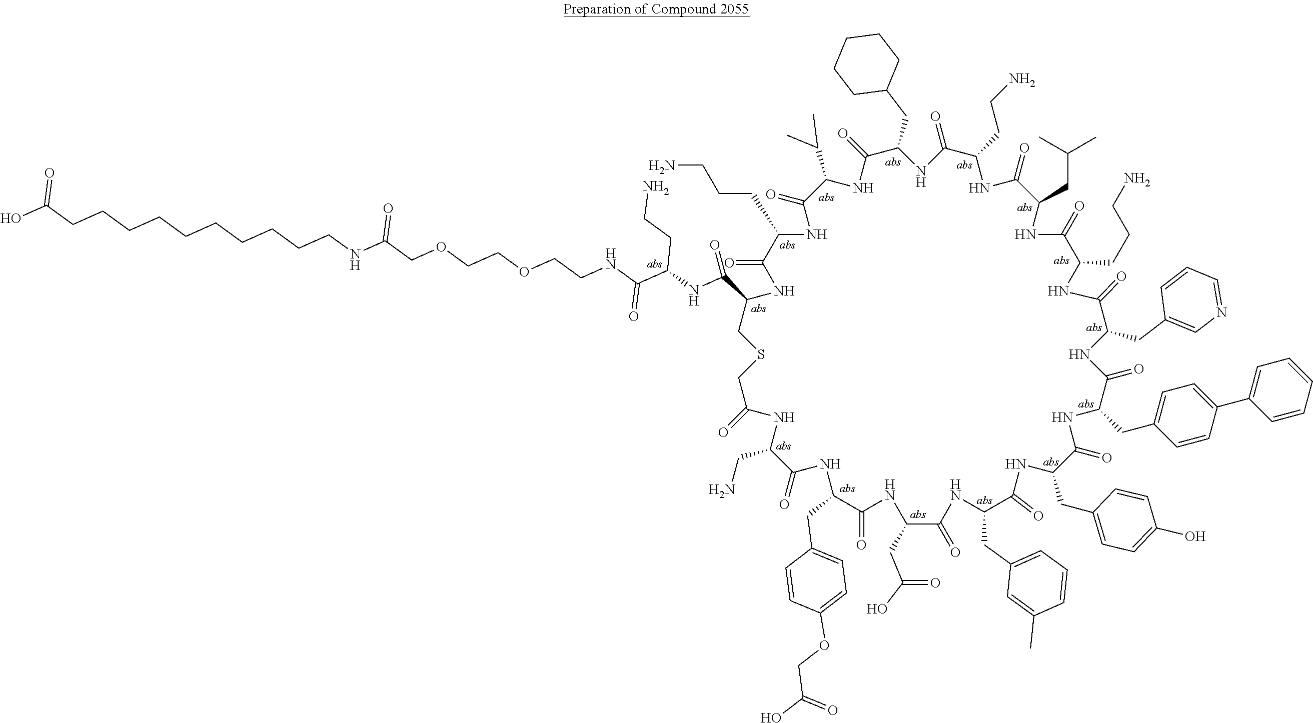 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3cccnc3)NC(=O)[C@H](Cc3ccc(-c4ccccc4)cc3)NC(=O)[C@H](Cc3ccc(O)cc3)NC2=O)c1]

Compound 2055 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Ala (3-pyridyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 31.4 mg, and its estimated purity by LCMS analysis was 91.8%. Analysis condition 1: Retention time=1.45 min; ESI-MS(+) m/z [M+2H]²⁺: 1201.2.

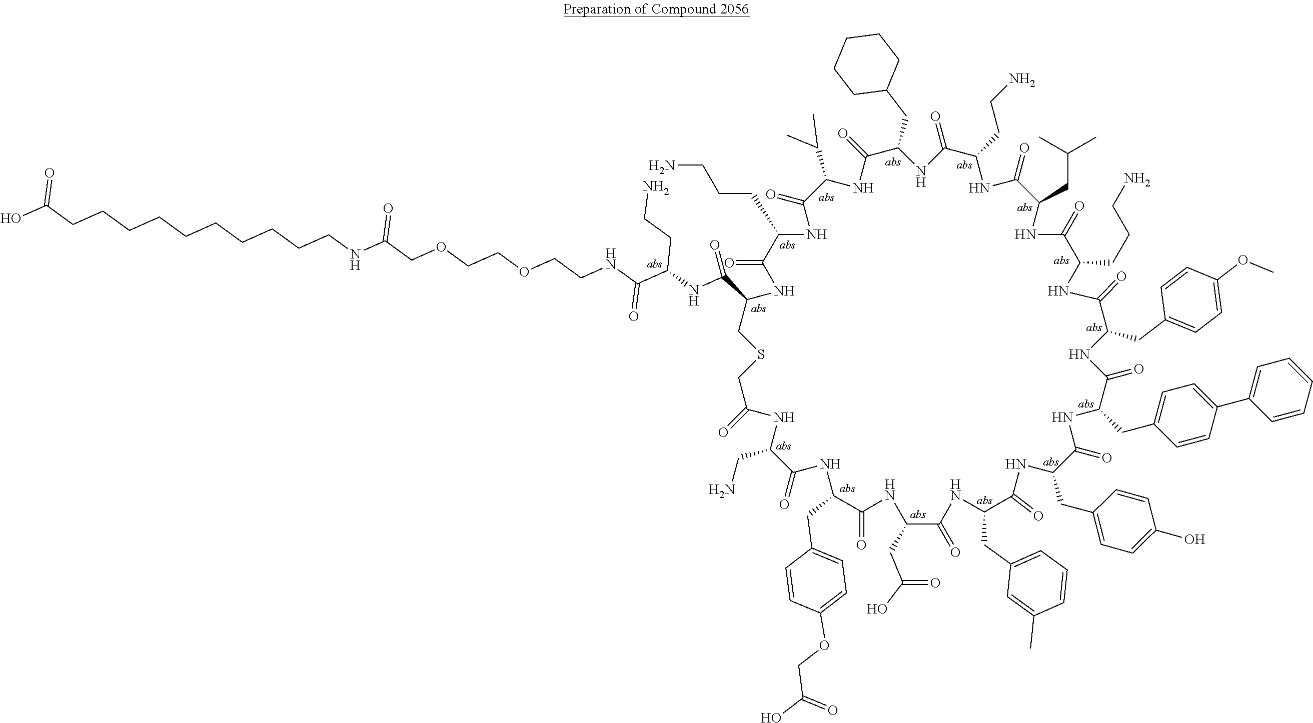 [SMILES: COc1ccc(C[C@@H]2NC(=O)[C@H](Cc3ccc(-c4ccccc4)cc3)NC(=O)[C@H](Cc3ccc(O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 2056 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.1 mg, and its estimated purity by LCMS analysis was 100%. Analysis condition 1: Retention time=1.53 min; ESI-MS(+) m/z [M+2H]²⁺: 1215.8.

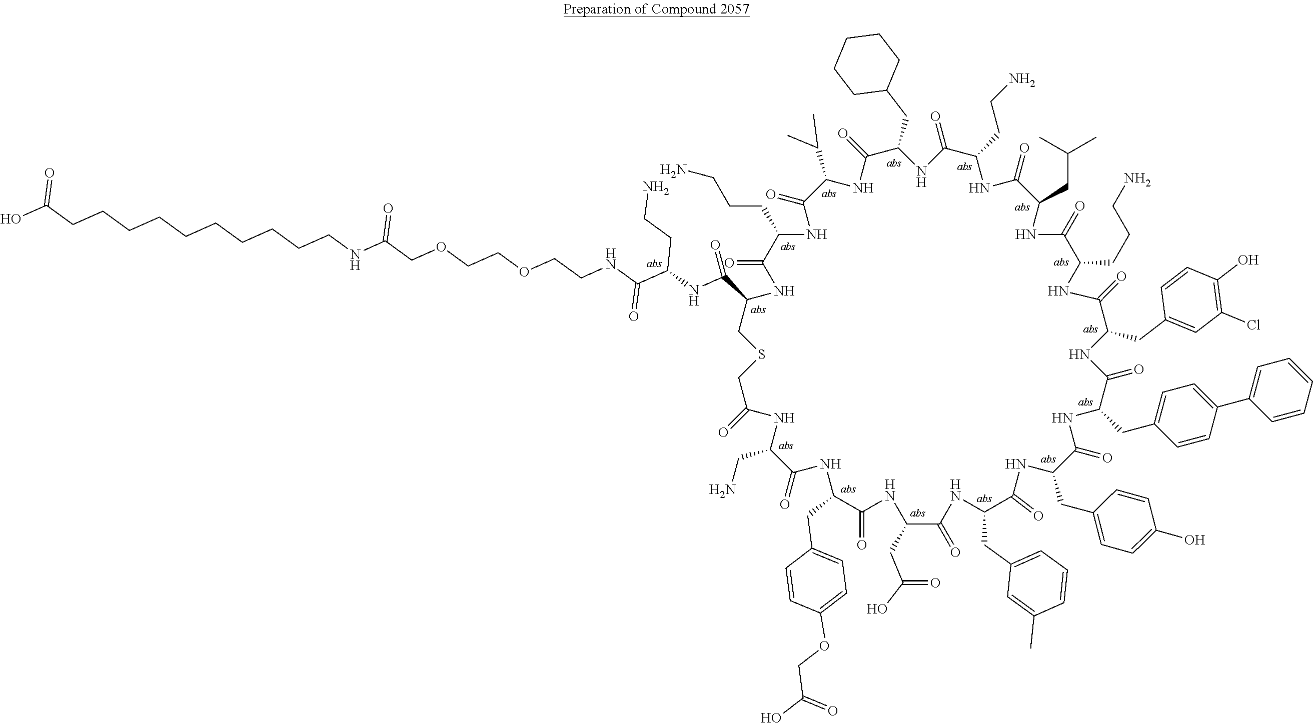 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)c(Cl)c3)NC(=O)[C@H](Cc3ccc(-c4ccccc4)cc3)NC(=O)[C@H](Cc3ccc(O)cc3)NC2=O)c1]

Compound 2057 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-C₁)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 21.2 mg, and its estimated purity by LCMS analysis was 100%. Analysis condition 2: Retention time=1.6 min; ESI-MS(+) m/z [M+2H]²⁺: 817.9.

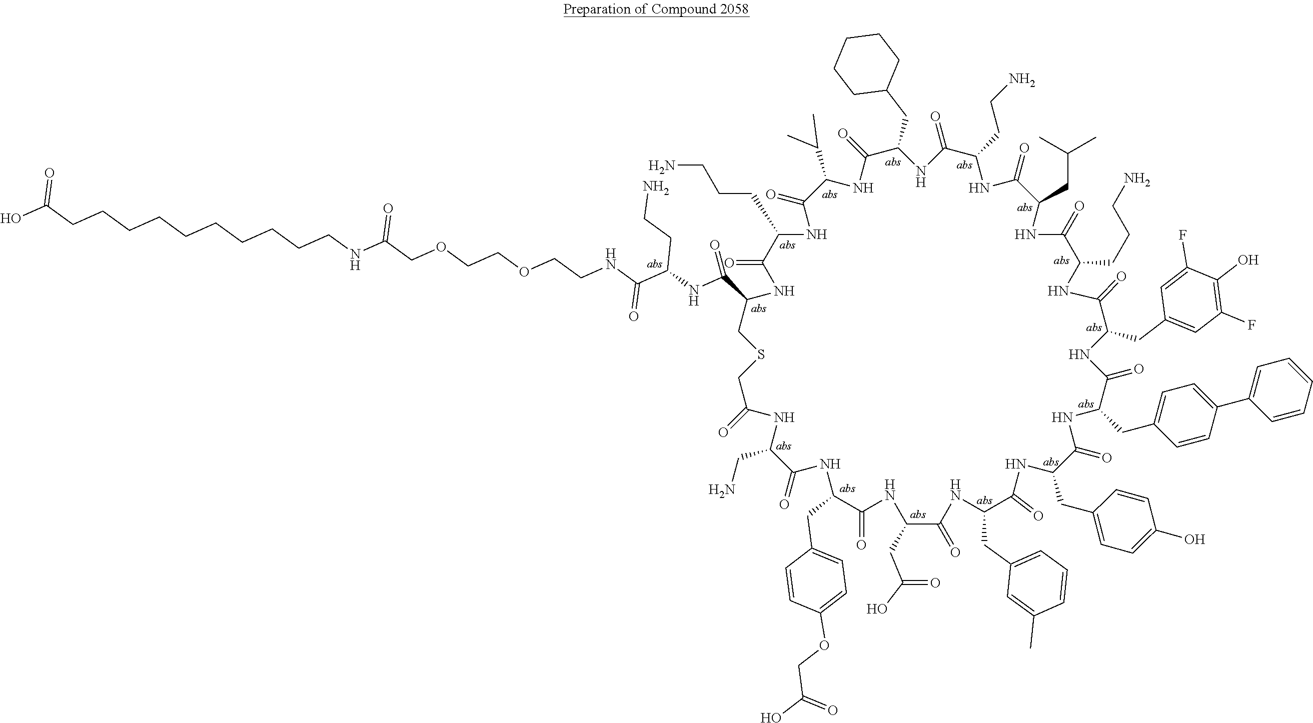 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3cc(F)c(O)c(F)c3)NC(=O)[C@H](Cc3ccc(-c4ccccc4)cc3)NC(=O)[C@H](Cc3ccc(O)cc3)NC2=O)c1]

Compound 2058 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(2,6-diF)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 23.2 mg, and its estimated purity by LCMS analysis was 100%. Analysis condition 1: Retention time=1.5 min; ESI-MS(+) m/z [M+2H]²⁺: 1226.9.

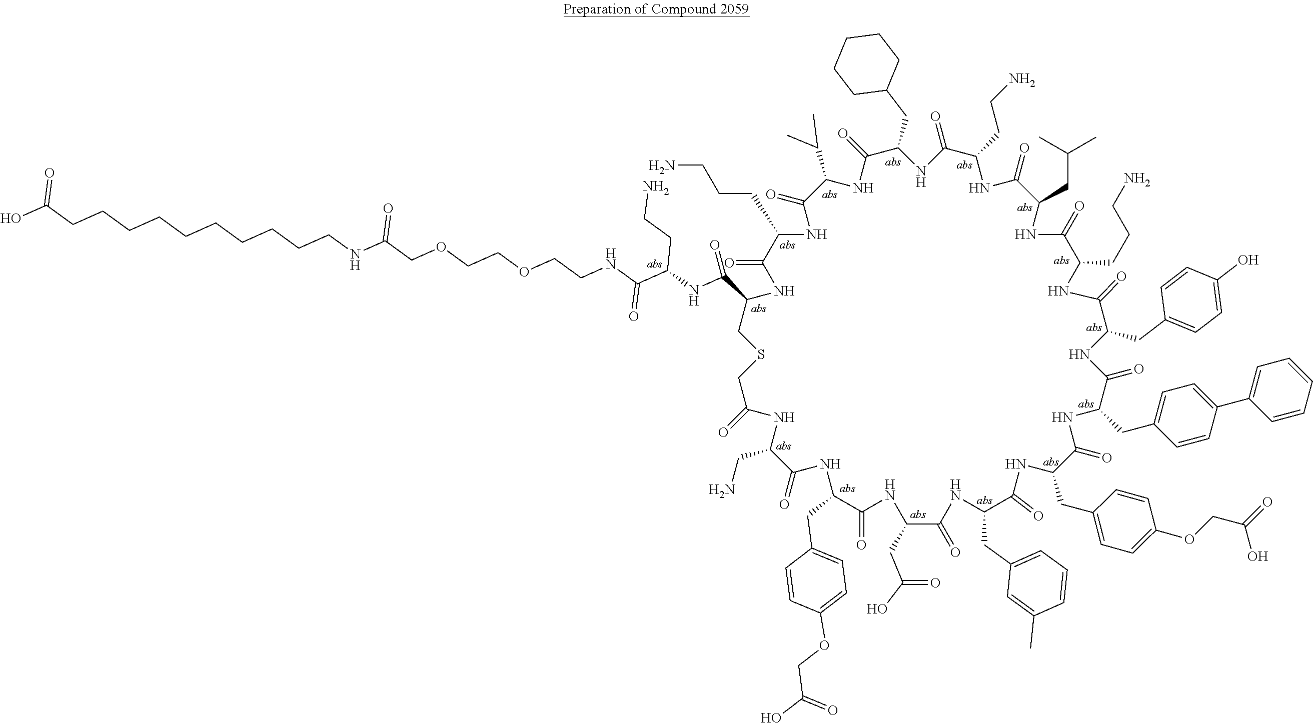 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Compound 2059 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21 mg, and its estimated purity by LCMS analysis was 100%. Analysis condition 1: Retention time=1.5 min; ESI-MS(+) m/z [M+2H]²⁺: 1238.

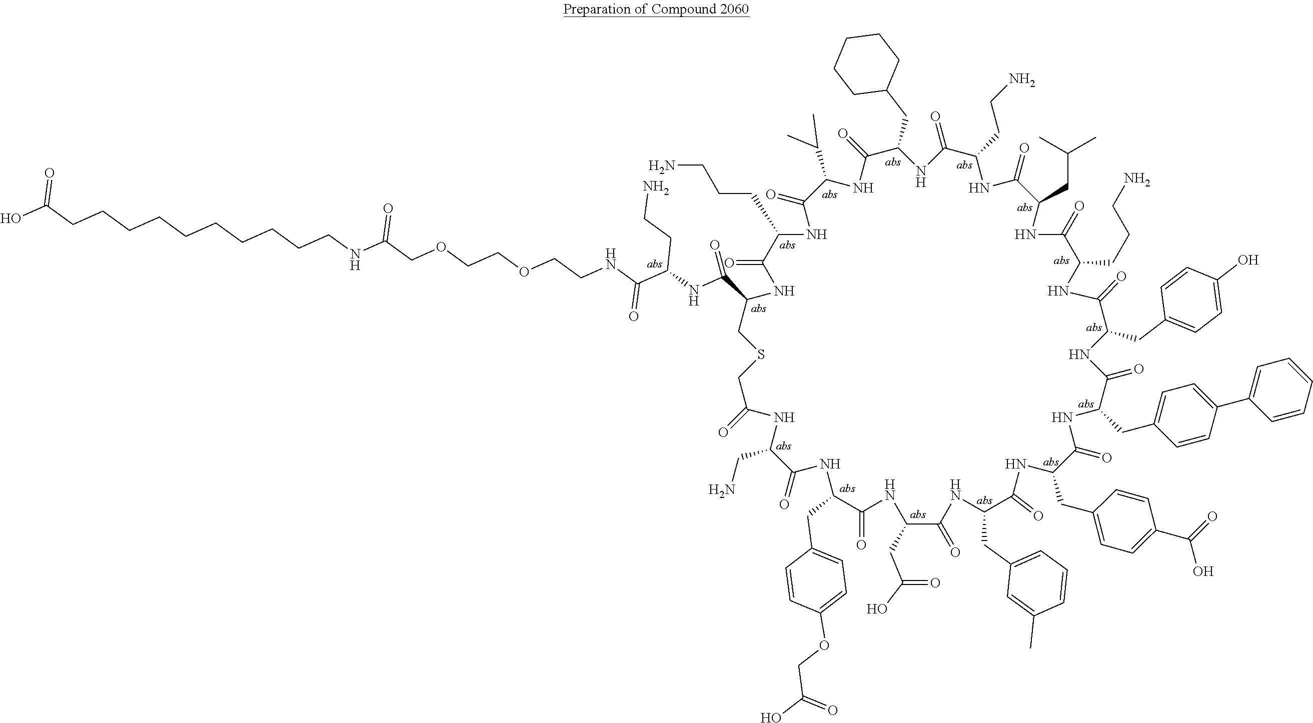 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(=O)O)cc3)NC2=O)c1]

Compound 2060 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 19.9 mg, and its estimated purity by LCMS analysis was 100%. Analysis condition 2: Retention time=1.58 min; ESI-MS(+) m/z [M+3H]³⁺: 816.

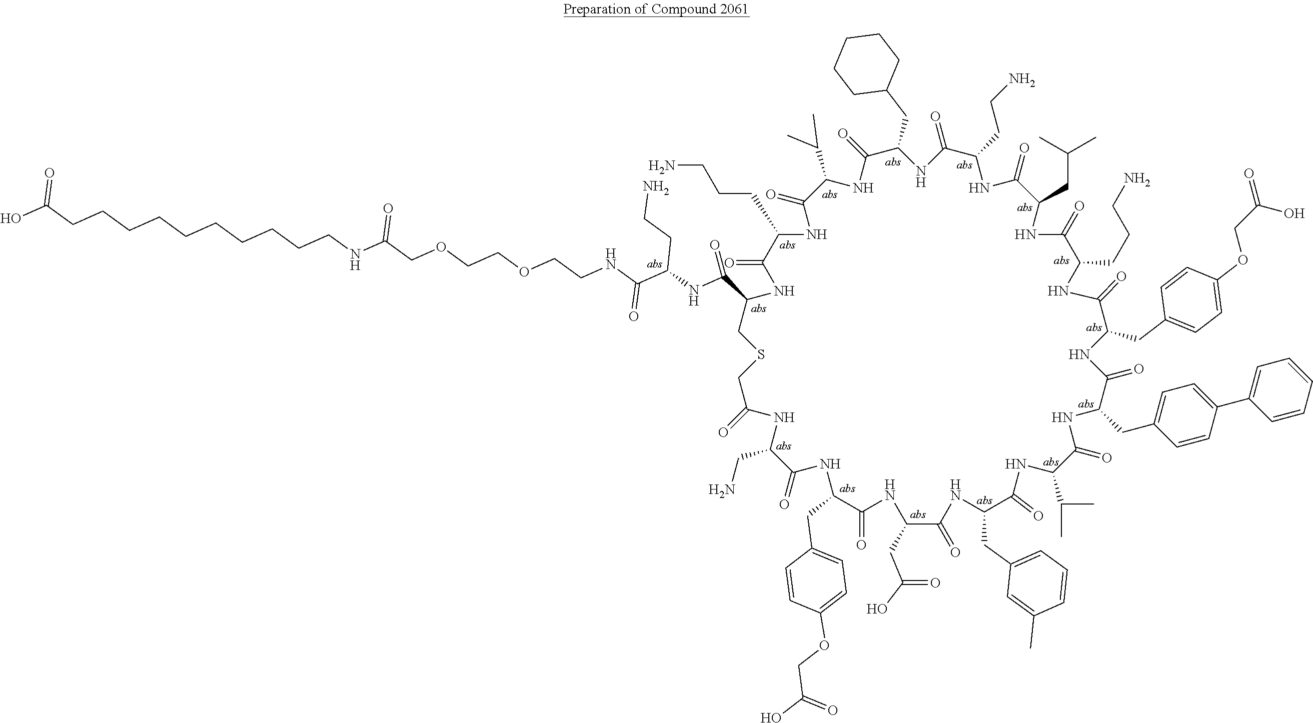 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 2061 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 33.1 mg, and its estimated purity by LCMS analysis was 100%. Analysis condition 1: Retention time=1.49 min; ESI-MS(+) m/z [M+2H]²⁺: 1206.1.

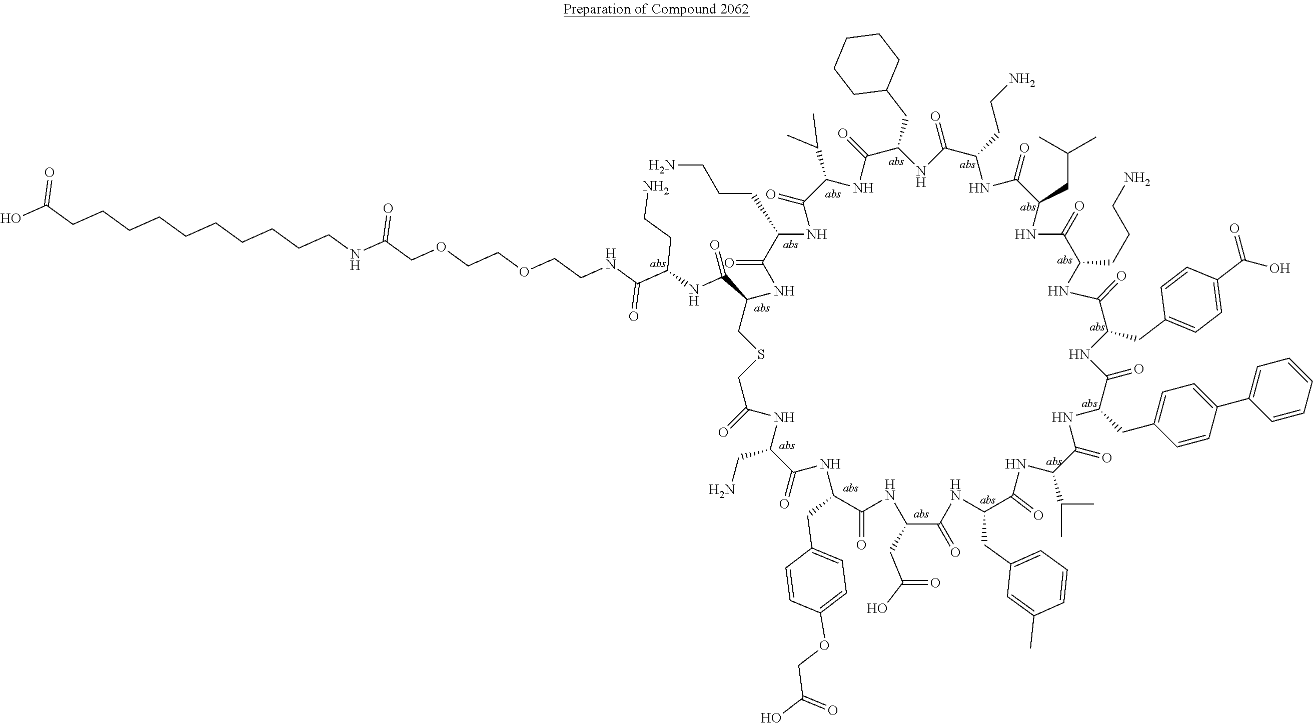 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](C(C)C)NC2=O)c1]

Compound 2062 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 36.8 mg, and its estimated purity by LCMS analysis was 93.8%. Analysis condition 1: Retention time=1.51 min; ESI-MS(+) m/z [M+2H]²⁺: 1191.1.

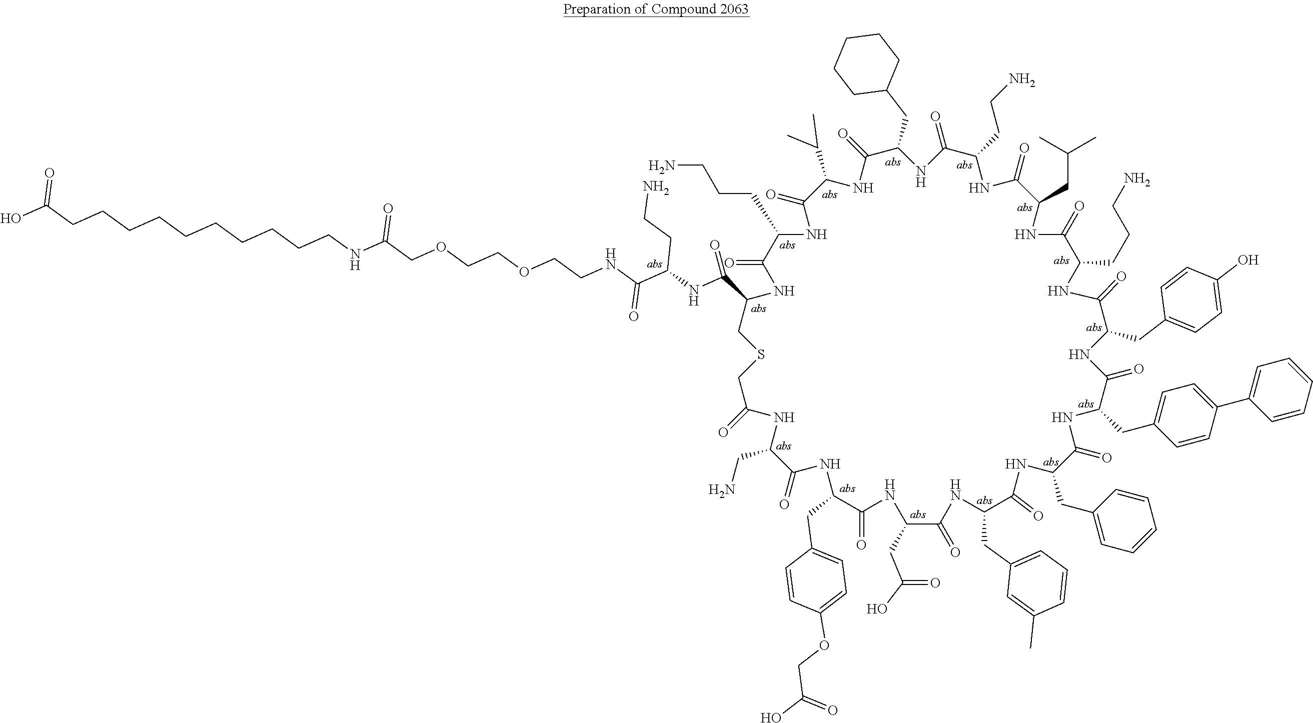 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccccc3)NC2=O)c1]

Compound 2063 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 48.4 mg, and its estimated purity by LCMS analysis was 100%. Analysis condition 1: Retention time=1.53 min; ESI-MS(+) m/z [M+2H]²⁺: 1201.3.

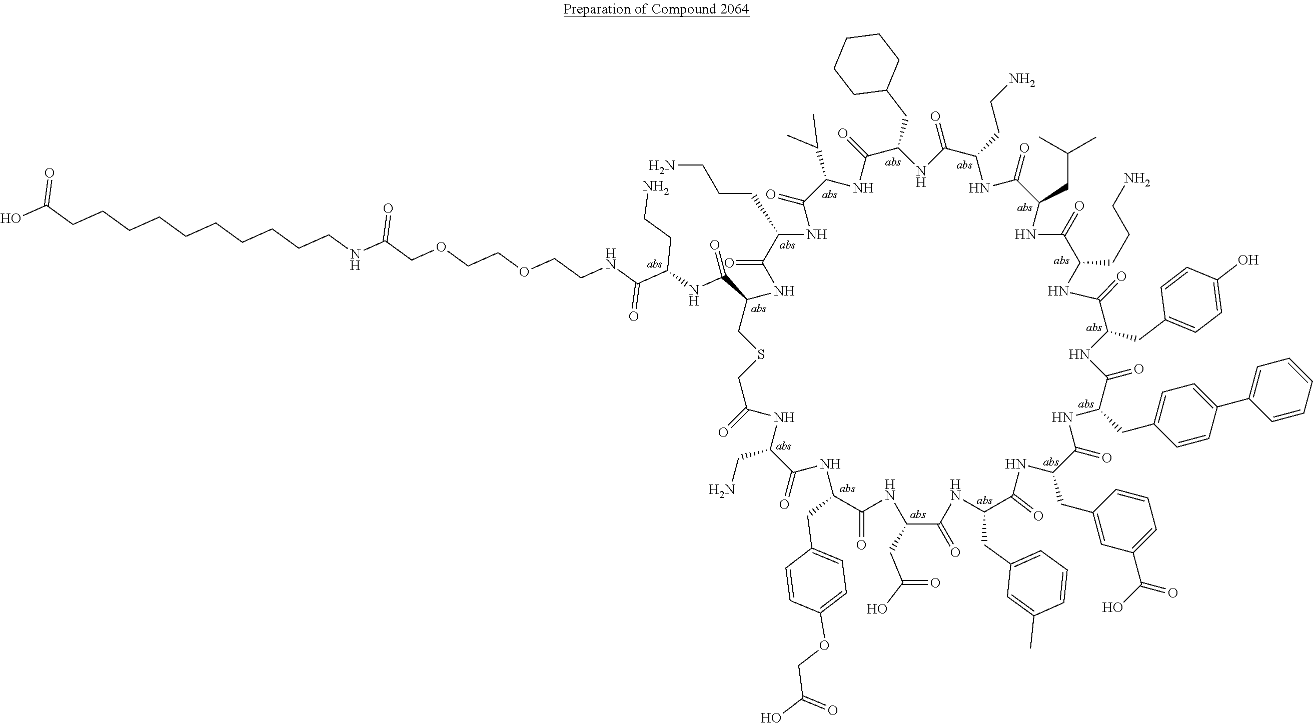 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3cccc(C(=O)O)c3)NC2=O)c1]

Compound 2064 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 20.6 mg, and its estimated purity by LCMS analysis was 96.2%. Analysis condition 1: Retention time=1.56 min; ESI-MS(+) m/z [M+3H]³⁺: 816.

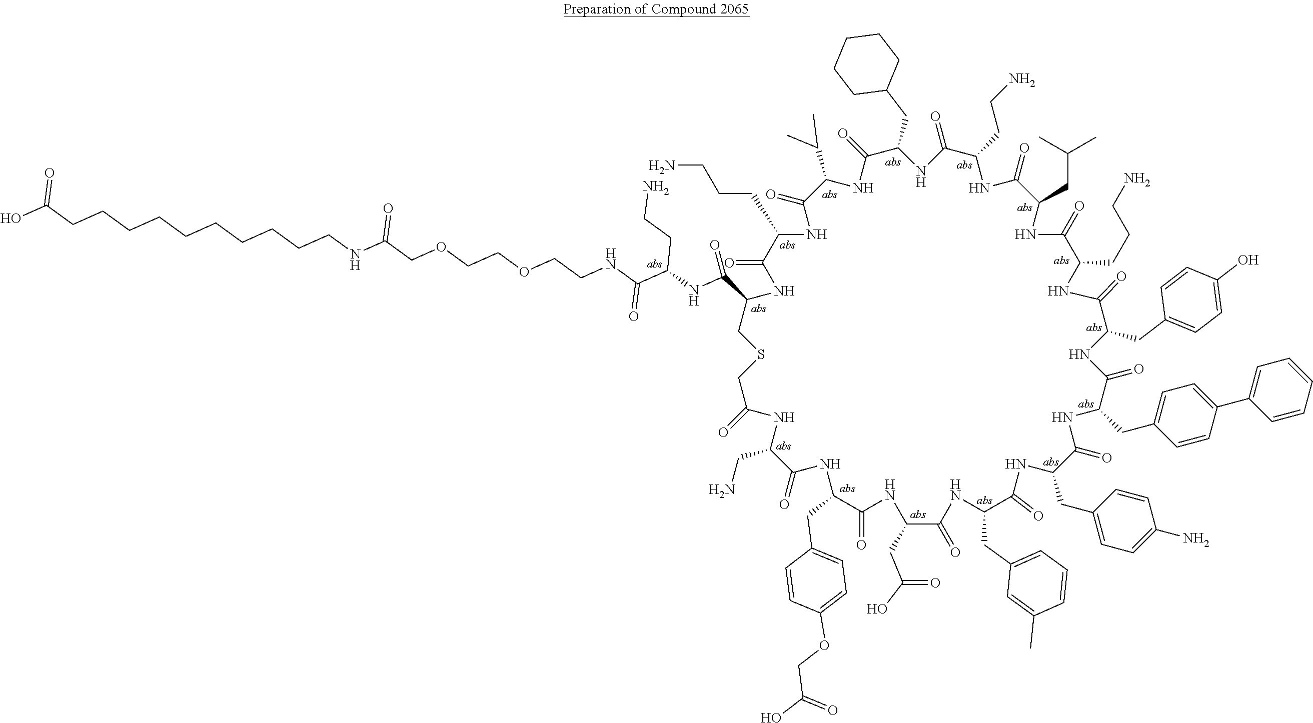 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(N)cc3)NC2=O)c1]

Compound 2065 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-NHBoc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.5 mg, and its estimated purity by LCMS analysis was 95.1%. Analysis condition 1: Retention time=1.49 min; ESI-MS(+) m/z [M+3H]²⁺: 806.

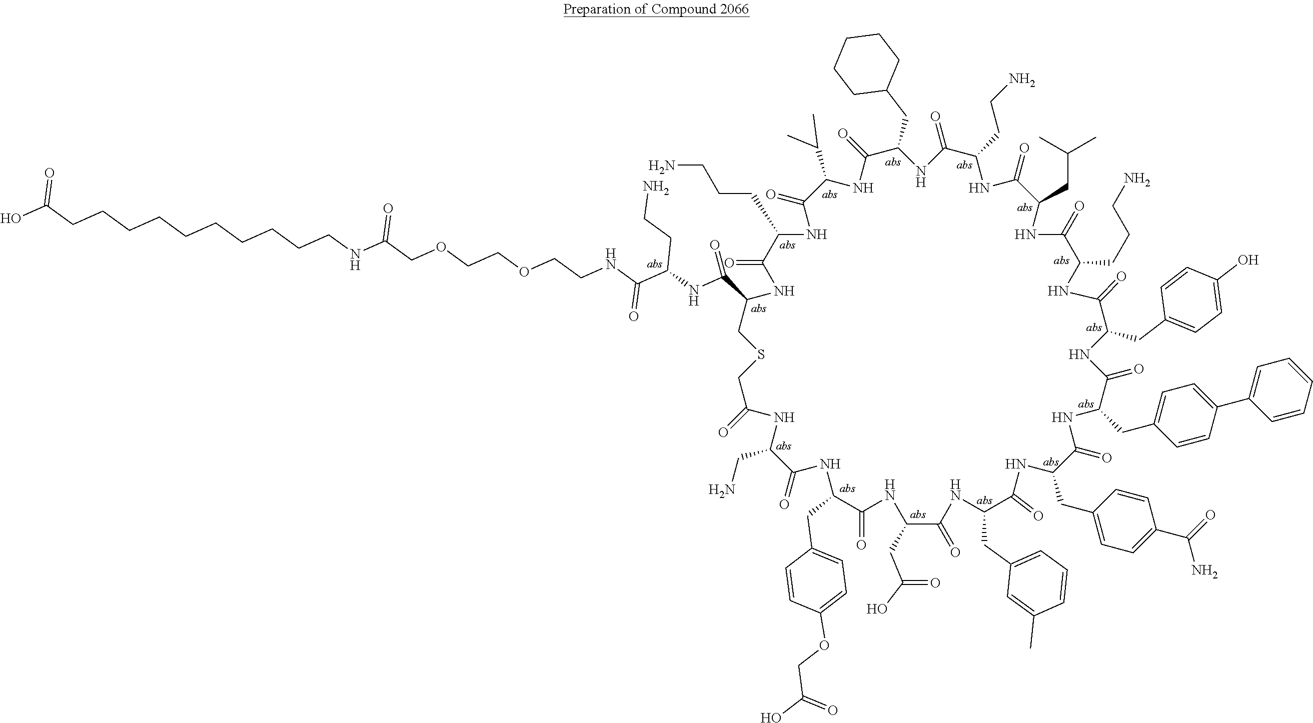 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 2066 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (40CONH₂)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 42.1 mg, and its estimated purity by LCMS analysis was 98.1%. Analysis condition 1: Retention time=1.49 min; ESI-MS(+) m/z [M+3H]³⁺: 815.8.

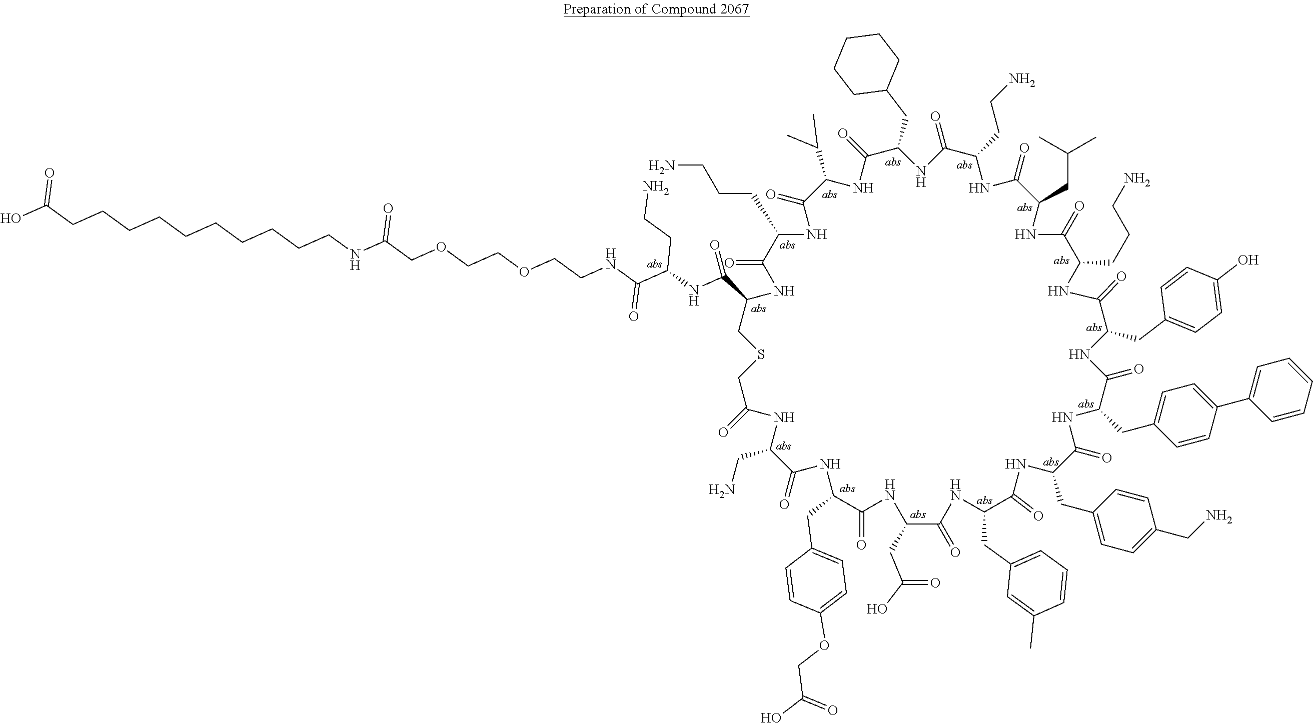 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(CN)cc3)NC2=O)c1]

Compound 2067 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-CH₂NHBoc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 9% B, 9-49% B over 26 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 73.2 mg, and its estimated purity by LCMS analysis was 100%. Analysis condition 2: Retention time=1.49 min; ESI-MS(+) m/z [M+3H]³⁺: 811.1.

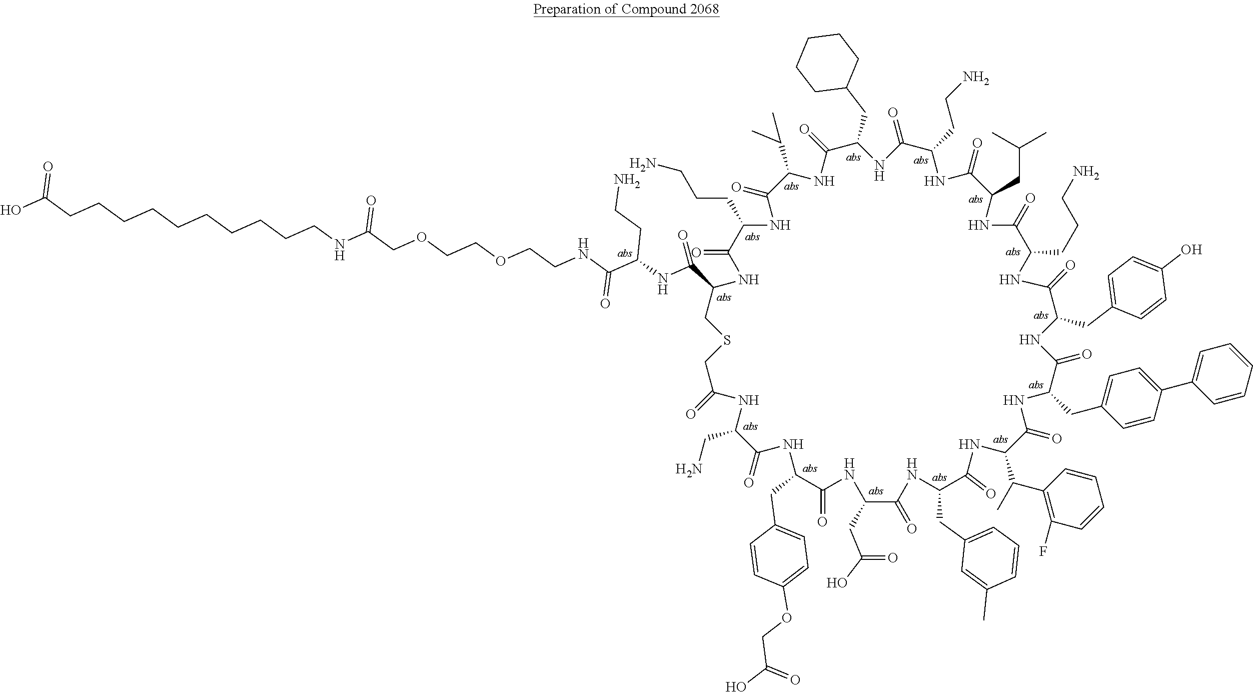 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)c3ccccc3F)NC2=O)c1]

Compound 2068 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (2-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 64.3 mg, and its estimated purity by LCMS analysis was 95.4%. Analysis condition 1: Retention time=1.6 min; ESI-MS(+) m/z [M+2H]²⁺: 1210.

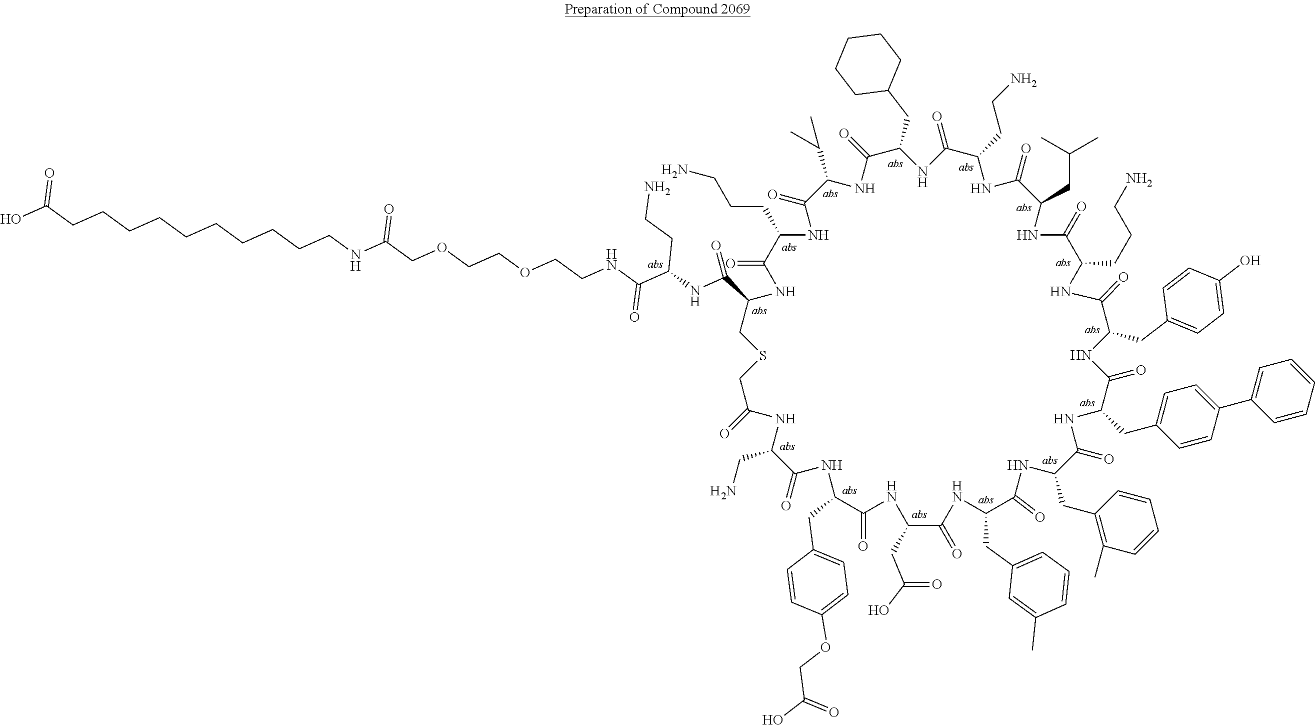 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccccc3C)NC2=O)c1]

Compound 2069 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (2-Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 48.8 mg, and its estimated purity by LCMS analysis was 98.3%. Analysis condition 2: Retention time=1.6 min; ESI-MS(+) m/z [M+2H]²⁺: 1208.2.

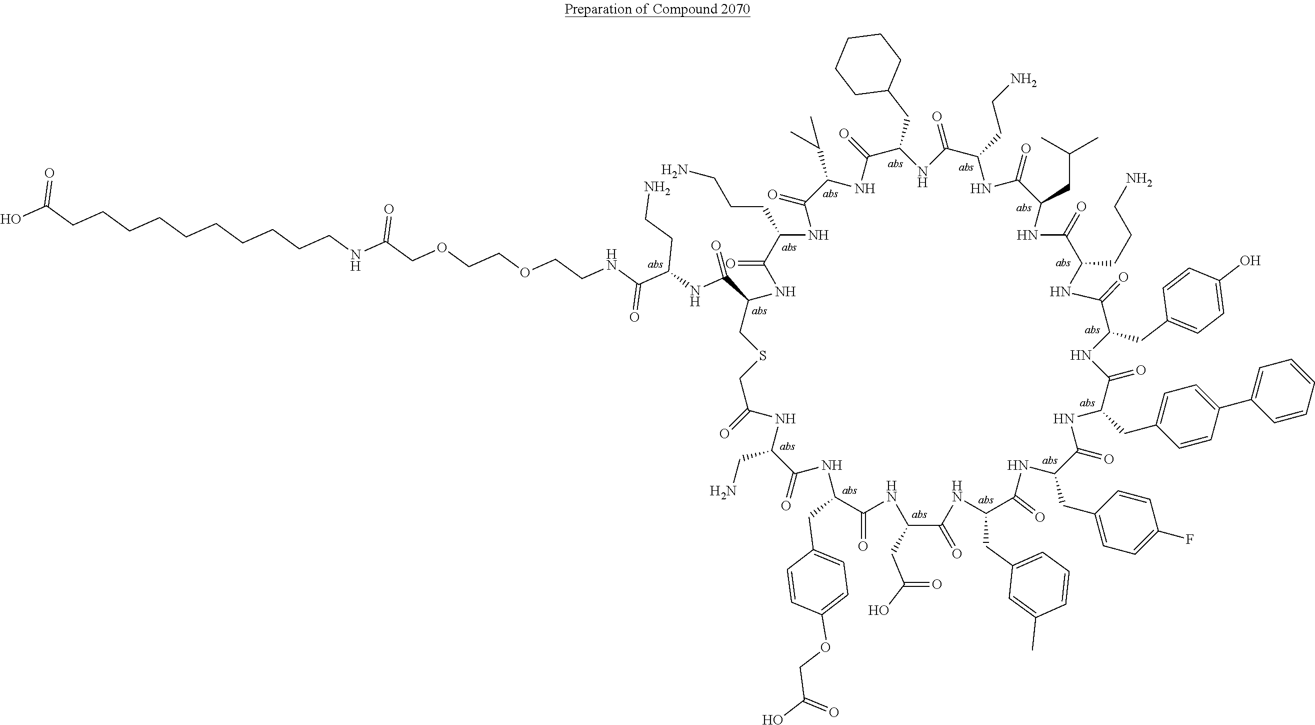 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(F)cc3)NC2=O)c1]

Compound 2070 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of composed of the following general procedures: Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 31 mg, and its estimated purity by LCMS analysis was 98.9%. Analysis condition 2: Retention time=1.55 min; ESI-MS(+) m/z [M+3H]³⁺: 807.

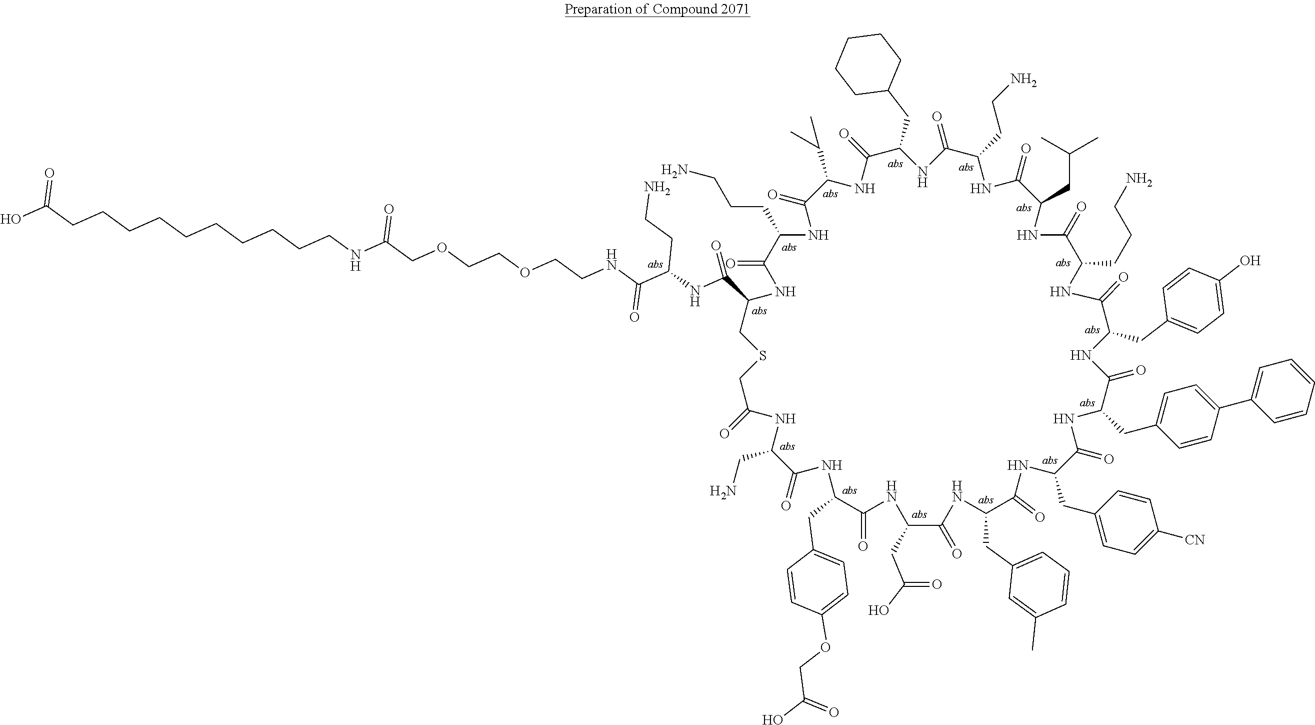 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C#N)cc3)NC2=O)c1]

Compound 2071 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-CN)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 0 mg, and its estimated purity by LCMS analysis was 85%. Analysis condition 1: Retention time=1.54 min; ESI-MS(+) m/z [M+3H]³⁺: 809.1.

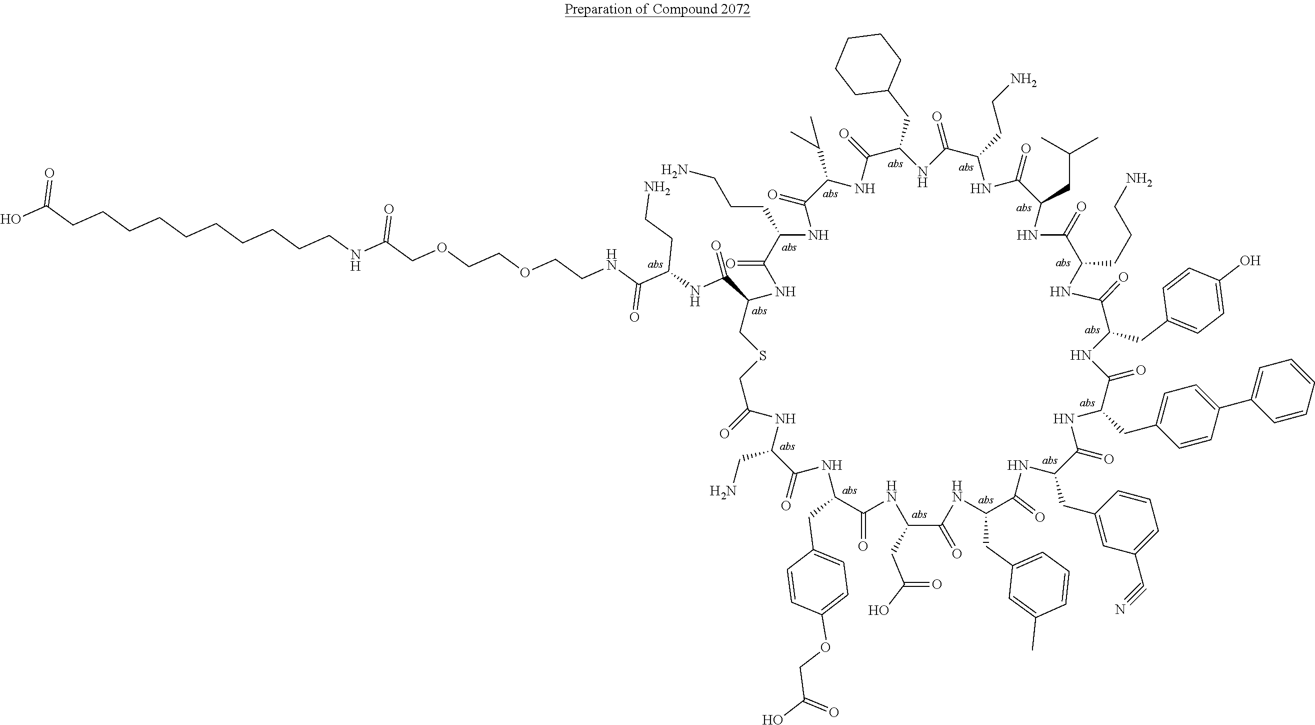 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3cccc(C#N)c3)NC2=O)c1]

Compound 2072 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CN)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 45.8 mg, and its estimated purity by LCMS analysis was 98.3%. Analysis condition 1: Retention time=1.55 min; ESI-MS(+) m/z [M+3H]³⁺: 809.1.

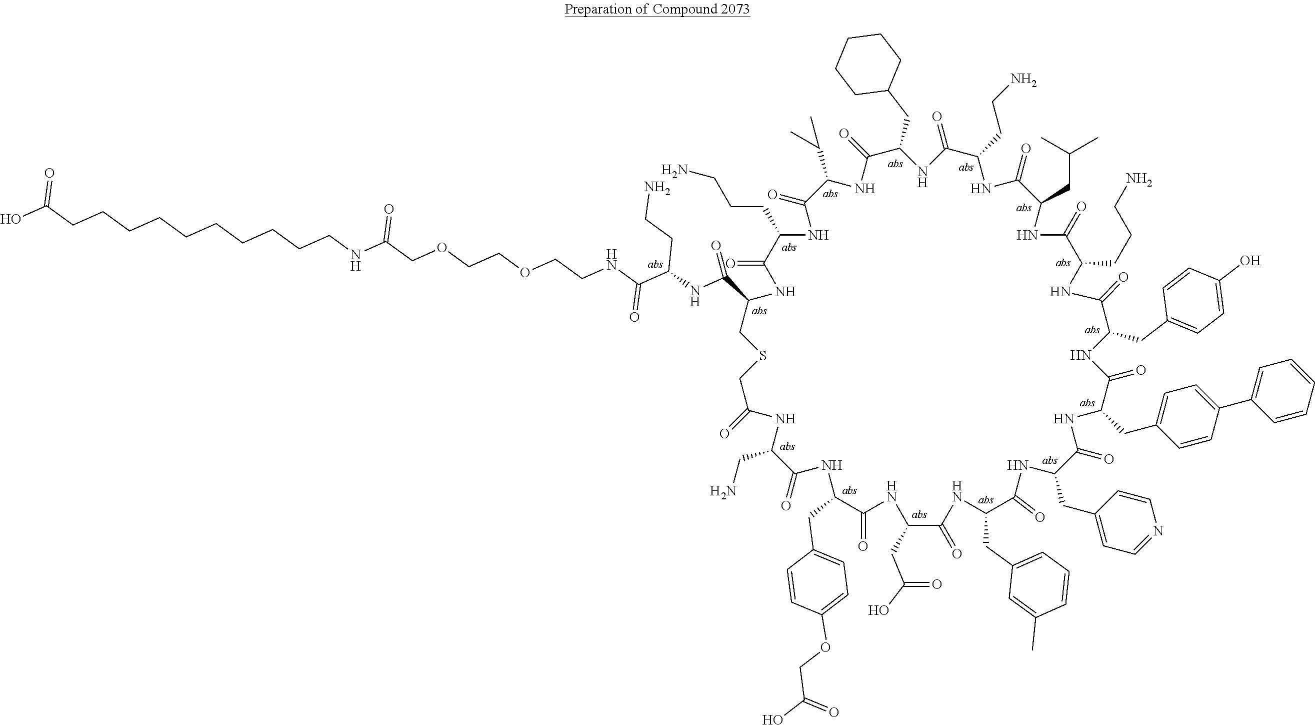 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccncc3)NC2=O)c1]

Compound 2073 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Ala (4-pyridyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-47% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 48.1 mg, and its estimated purity by LCMS analysis was 96.1%. Analysis condition 2: Retention time=1.43 min; ESI-MS(+) m/z [M+2H]²⁺: 1202.

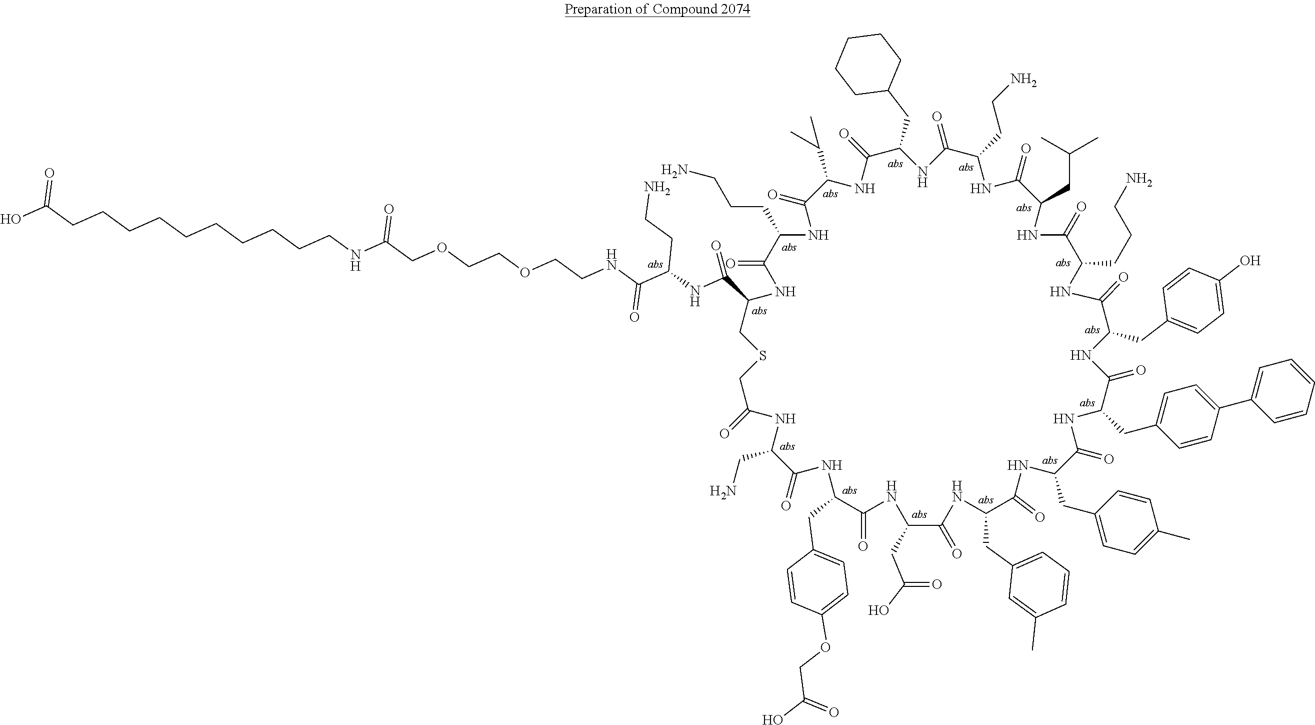 [SMILES: Cc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC2=O)cc1]

Compound 2074 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of composed of the following general procedures Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 31.8 mg, and its estimated purity by LCMS analysis was 95%. Analysis condition 1: Retention time=1.54 min; ESI-MS(+) m/z [M+2H]²⁺: 1208.1.

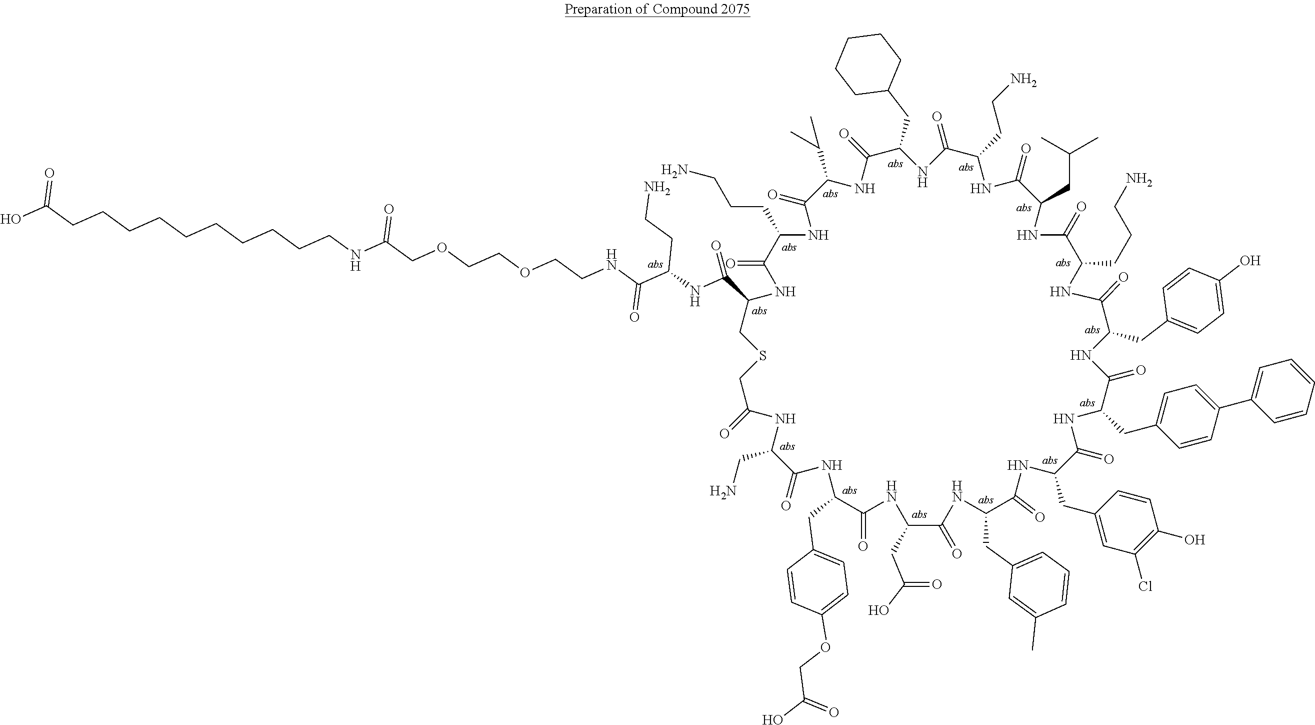 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)c(Cl)c3)NC2=O)c1]

Compound 2075 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-Cl)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.8 mg, and its estimated purity by LCMS analysis was 96.8%. Analysis condition 1: Retention time=1.56 min; ESI-MS(+) m/z [M+3H]³⁺: 818.1.

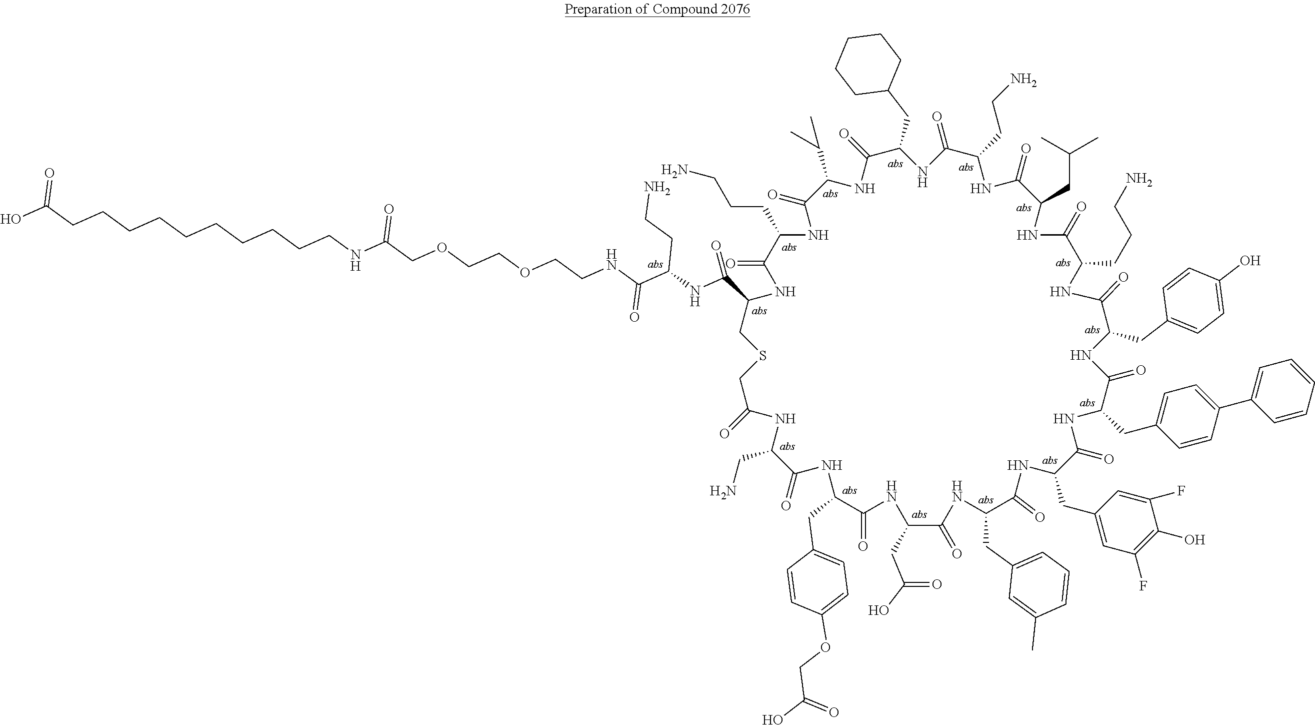 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3cc(F)c(O)c(F)c3)NC2=O)c1]

Compound 2076 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(2,6-diF)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 21.2 mg, and its estimated purity by LCMS analysis was 97.2%. Analysis condition 1: Retention time=1.57 min; ESI-MS(+) m/z [M+2H]²⁺: 1227.

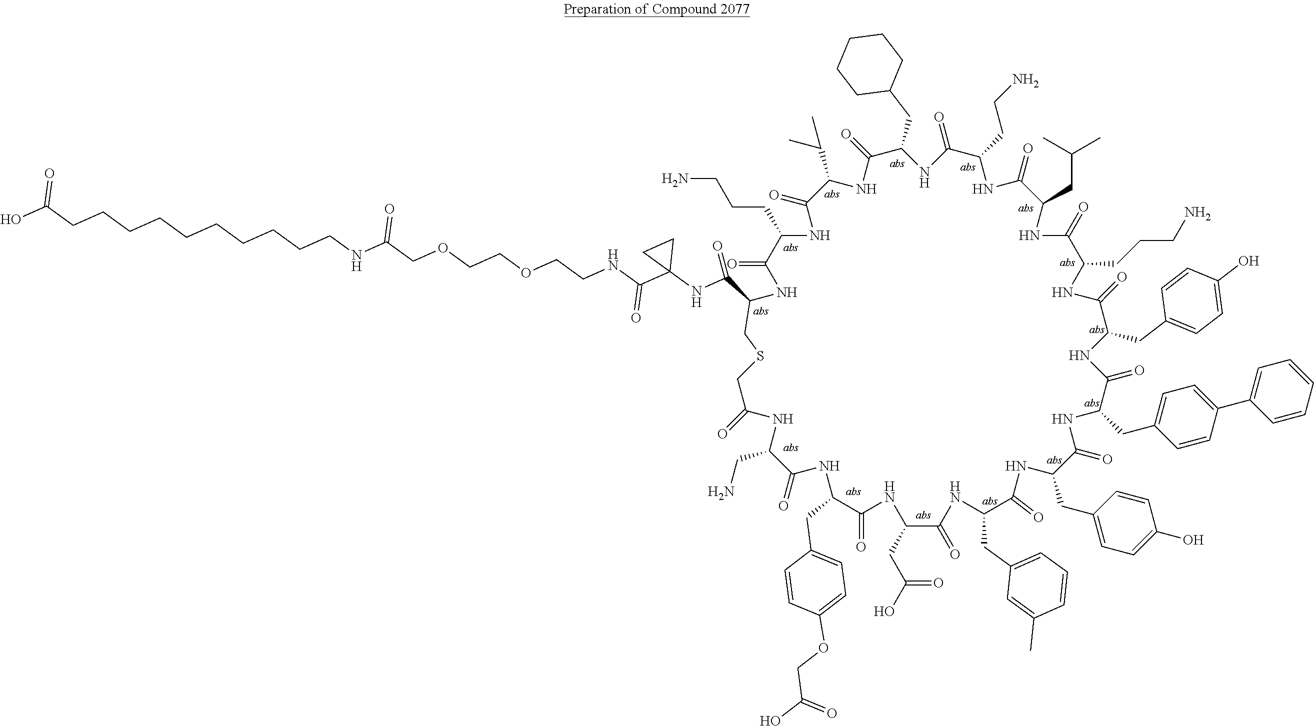 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NC3(C(=O)NCCOCCOCC(=O)NCCCCCCCCCCC(=O)O)CC3)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2077 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 108.6 mg, and its estimated purity by LCMS analysis was 95.4%. Analysis condition 1: Retention time=1.59 min; ESI-MS(+) m/z [M+3H]³⁺: 801.

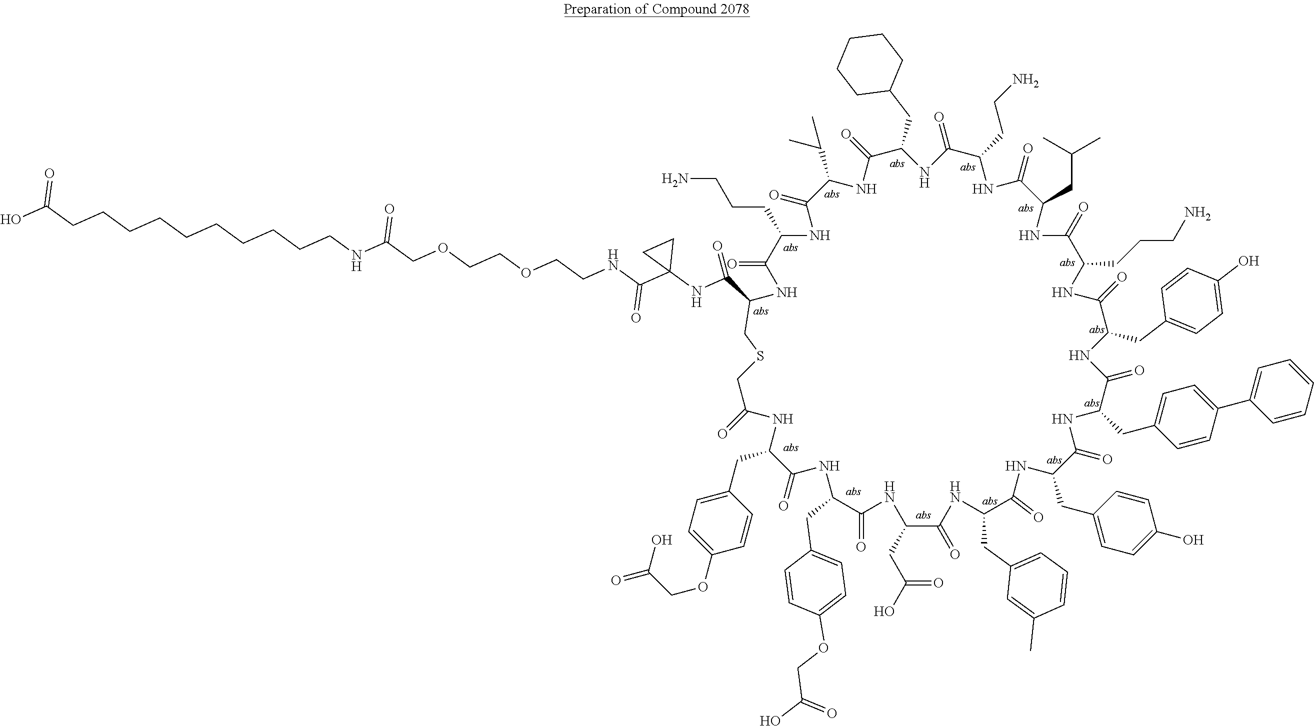 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NC3(C(=O)NCCOCCOCC(=O)NCCCCCCCCCCC(=O)O)CC3)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2078 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 68.2 mg, and its estimated purity by LCMS analysis was 97.5%. Analysis condition 2: Retention time=1.68 min; ESI-MS(+) m/z [M+2H]²⁺: 1268.2.

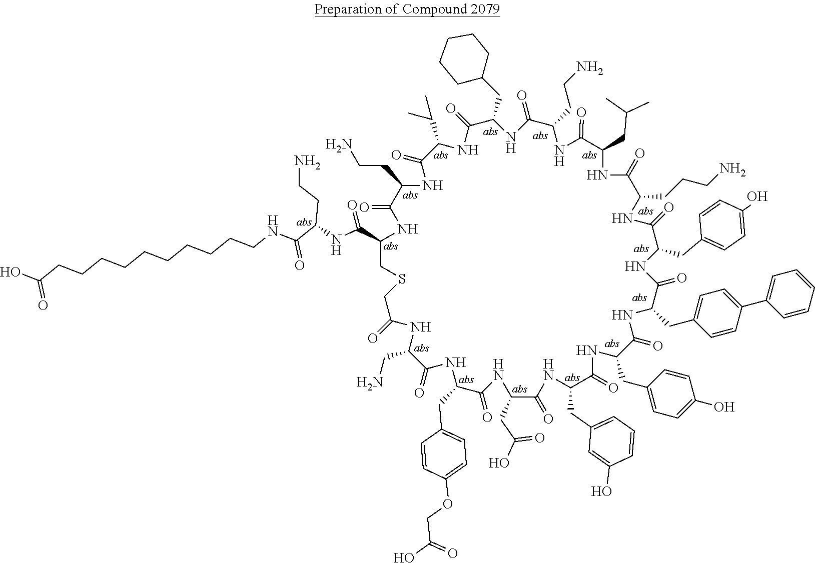 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2cccc(O)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@@H](CCN)NC(=O)[C@H](C(C)C)NC(=O)[C@H](CC2CCCCC2)NC(=O)[C@H](CCN)NC1=O]

Compound 2079 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The yield of the product was mg, and its estimated purity by LCMS analysis was %. Analysis condition: Retention time=1.53 min; ESI-MS(+) m/z [M+2H]²⁺:

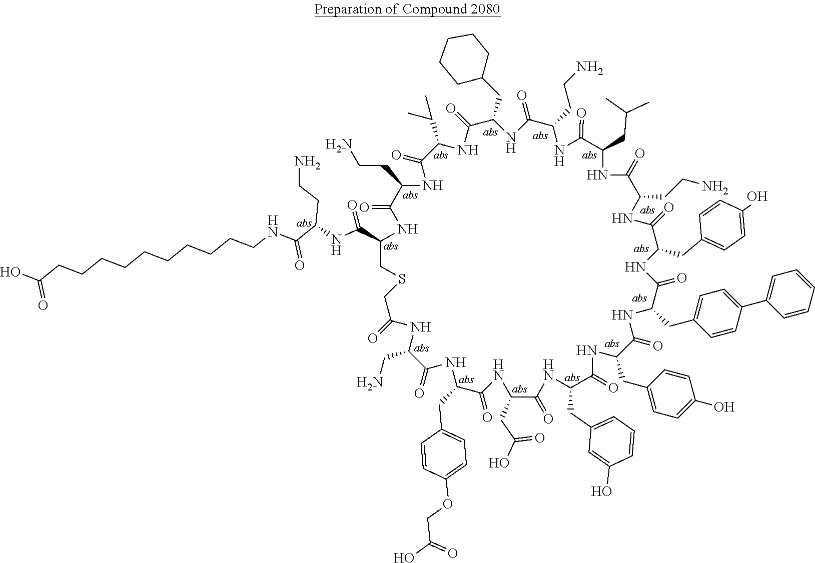 [SMILES: CC(C)C[C@H]1NC(=O)[C@H](CCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2cccc(O)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@@H](CCN)NC(=O)[C@H](C(C)C)NC(=O)[C@H](CC2CCCCC2)NC(=O)[C@H](CCN)NC1=O]

Compound 2080 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 23.3 mg, and its estimated purity by LCMS analysis was 87.6%. Analysis condition 1: Retention time=1.34 min; ESI-MS(+) m/z [M+2H]²⁺: 1123.9.

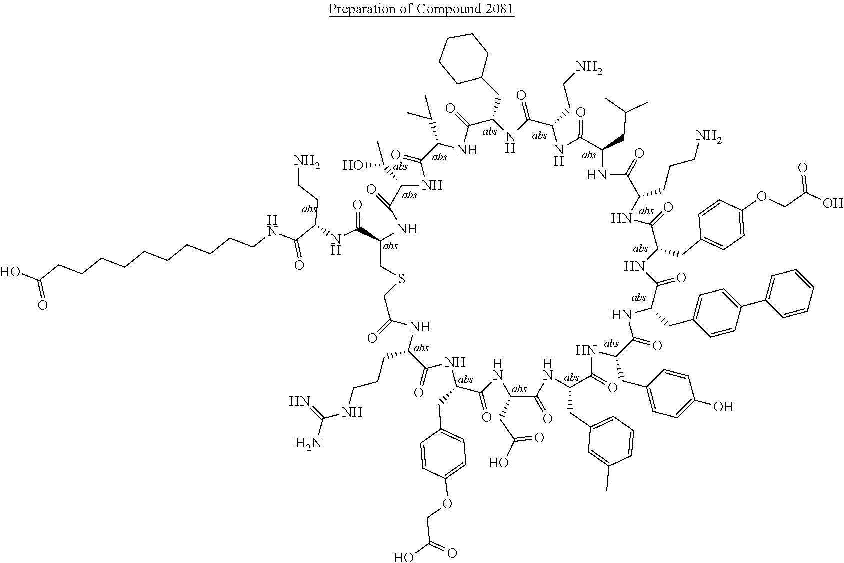 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2081 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by UV signals. Fractions containing the desired product were combined and dried via centrifugal evaporation. The yield of the product was 29.2 mg, and its estimated purity by LCMS analysis was 94.8% as a mixture of diastereomers. Analysis condition 2: Retention time=1.53, 1.57 min; ESI-MS(+) m/z [M+2H]²⁺: 1194.12, 1194.26.

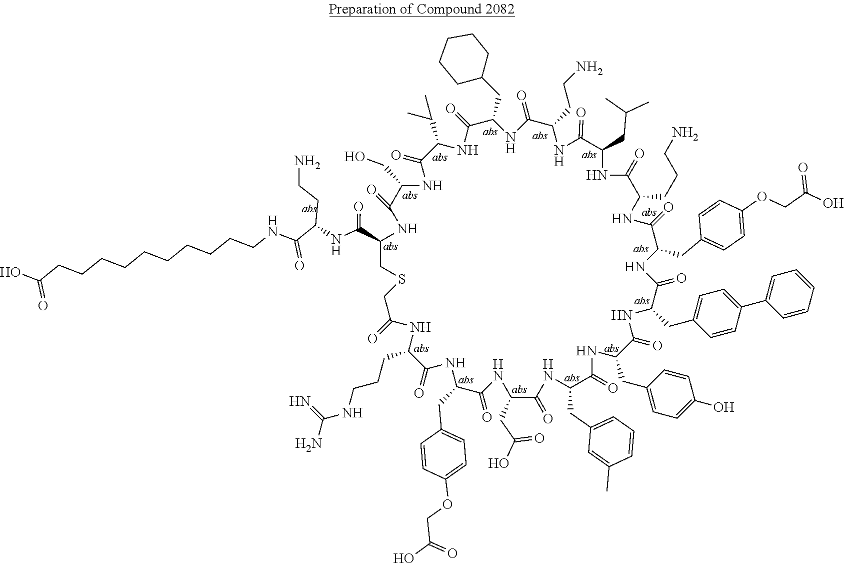 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2082 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9 mg, and its estimated purity by LCMS analysis was 97.8%. Analysis condition 1: Retention time=1.58 min; ESI-MS(+) m/z [M+3H]³⁺: 792.

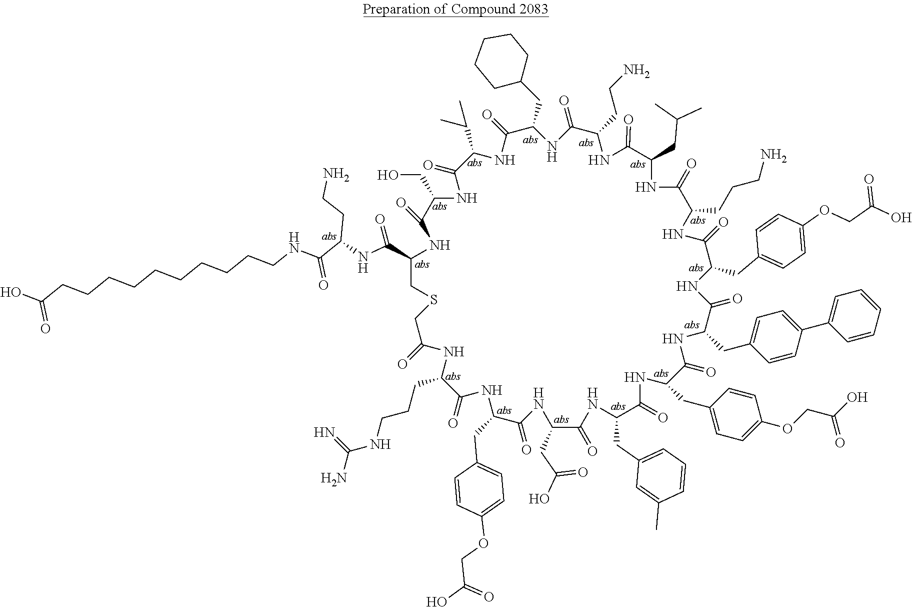

Compound 2083 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 10% B, 10-50% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 4.7 mg, and its estimated purity by LCMS analysis was 100%. Analysis condition 1: Retention time=1.63 min; ESI-MS(+) m/z [M+2H]²⁺: 1216.1.

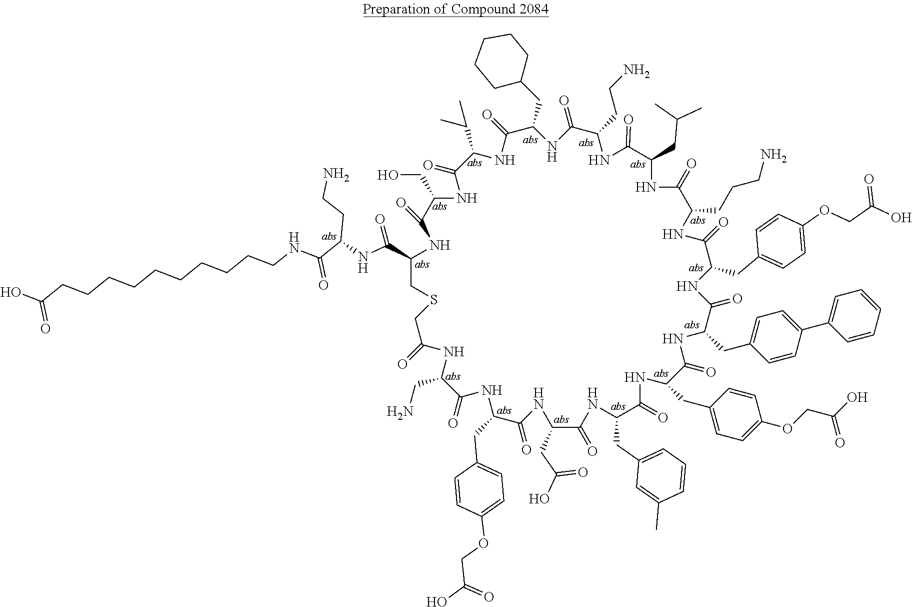

Compound 2084 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-45% B over 35 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.5 mg, and its estimated purity by LCMS analysis was 97.1%. Analysis condition 2: Retention time=1.51 min; ESI-MS(+) m/z [M+2H]²⁺: 1181.1.

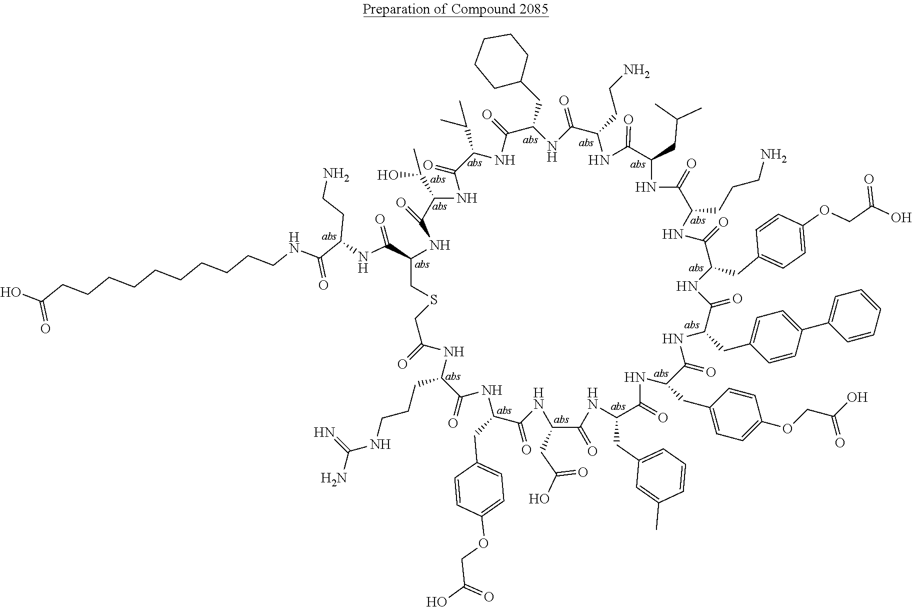

Compound 2085 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 1.8 mg, and its estimated purity by LCMS analysis was 95.2%. Analysis condition 1: Retention time=1.56 min; ESI-MS(+) m/z [M+2H]²⁺: 1223.1.

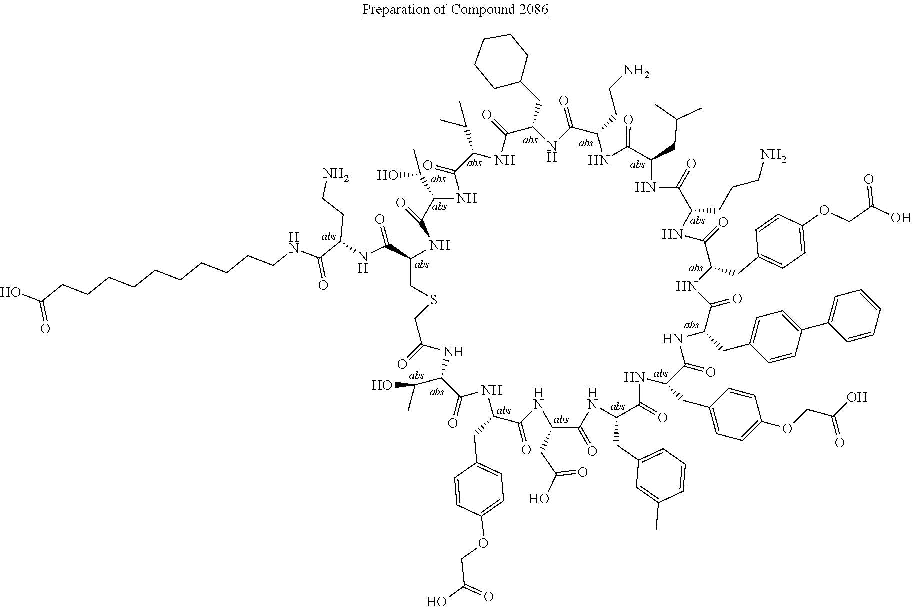

Compound 2086 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 22 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 3-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4.4 mg, and its estimated purity by LCMS analysis was 95.5%. Analysis condition 2: Retention time=1.47 min; ESI-MS(+) m/z [M+3H]³⁺: 797.3.

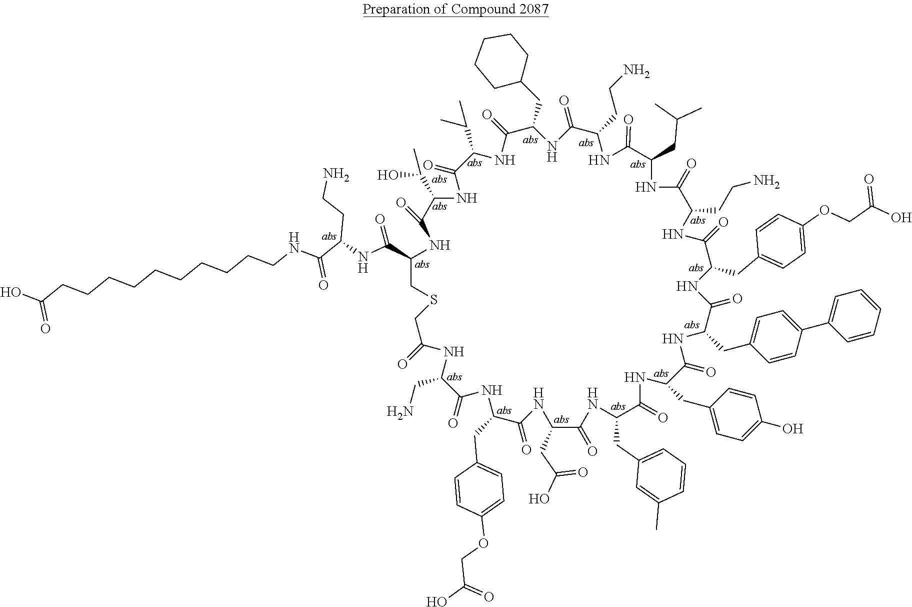

Compound 2087 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 11.9 mg, and its estimated purity by LCMS analysis was 95.1%. Analysis condition 1: Retention time=1.64 min; ESI-MS(+) m/z [M+2H]²⁺: 1152.

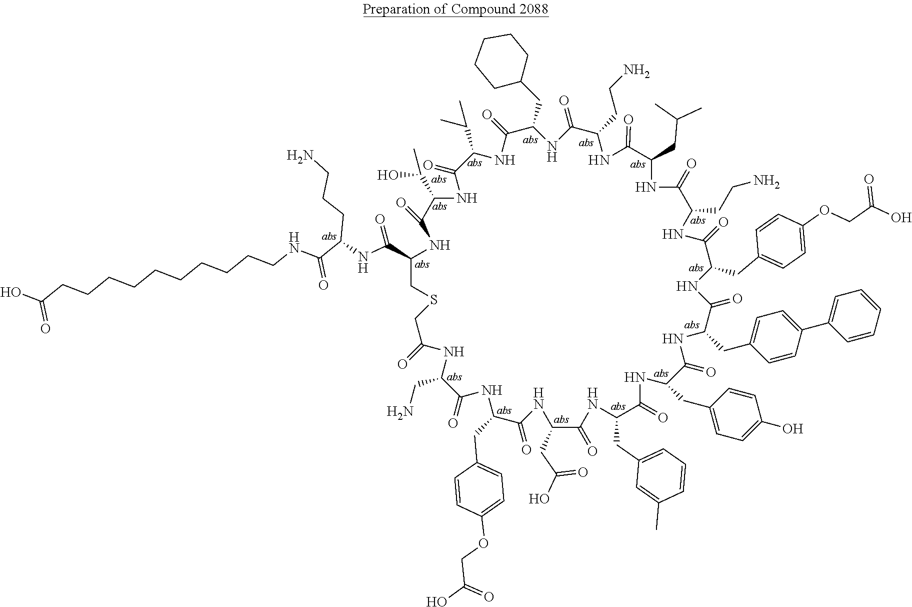

Compound 2088 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 31.3 mg, and its estimated purity by LCMS analysis was 86.9%. Analysis condition 2: Retention time=1.57 min; ESI-MS(+) m/z [M+2H]²⁺: 1159.

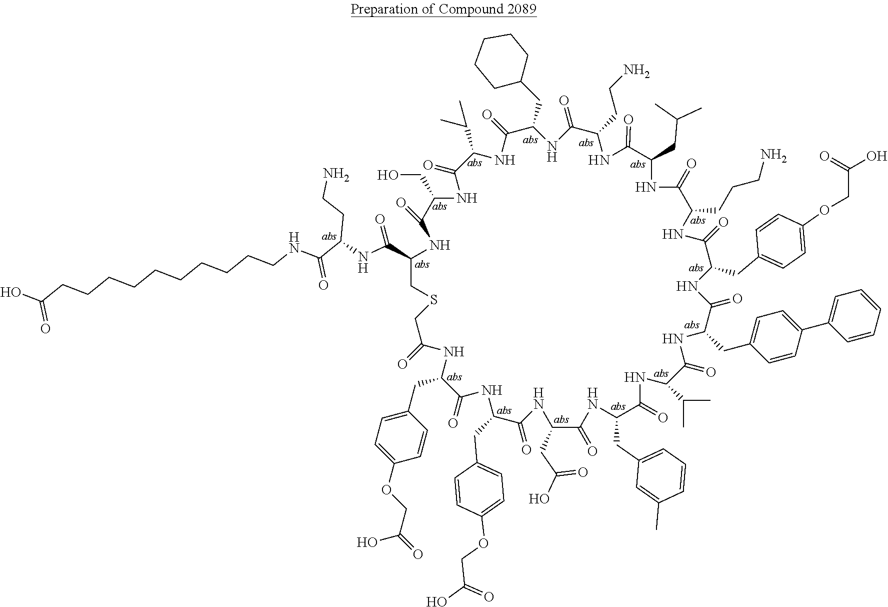

Compound 2089 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 7% B, 7-47% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.3 mg, and its estimated purity by LCMS analysis was 95.7%. Analysis condition 2: Retention time=1.29 min; ESI-MS(+) m/z [M+2H]²⁺: 1187.5.

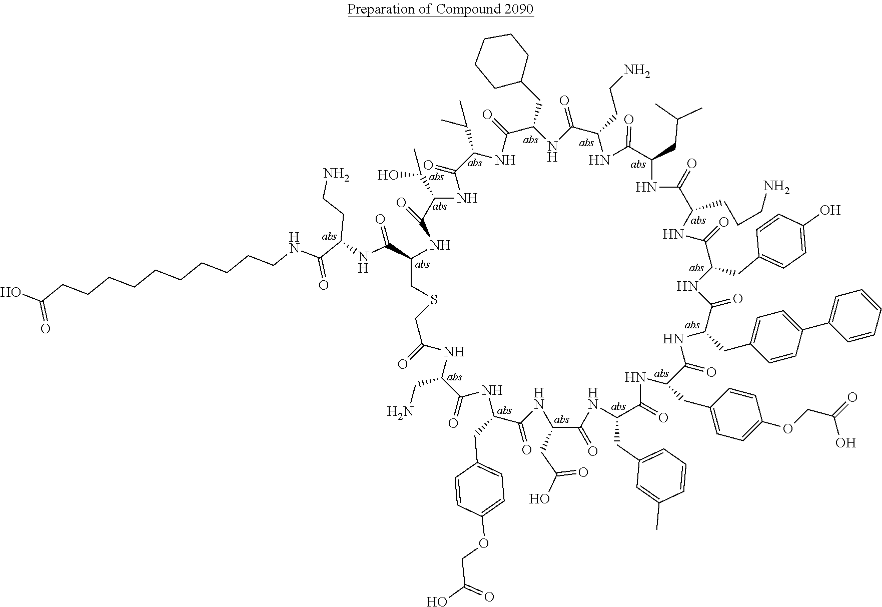

Compound 2090 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20.2 mg, and its estimated purity by LCMS analysis was 91.1% as a mixture of diastereomers. Analysis condition 2: Retention time=1.57, 1.61 min; ESI-MS(+) m/z [M+2H]²⁺: 1159.15, 1159.15.

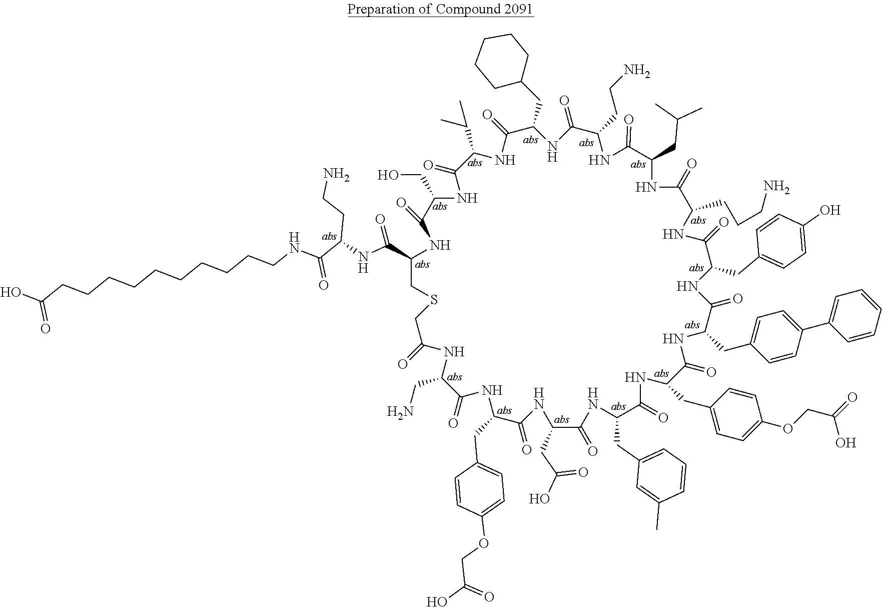

Compound 2091 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.2 mg, and its estimated purity by LCMS analysis was 88.4%. Analysis condition 2: Retention time=1.55 min; ESI-MS(+) m/z [M+2H]²⁺: 1152.2.

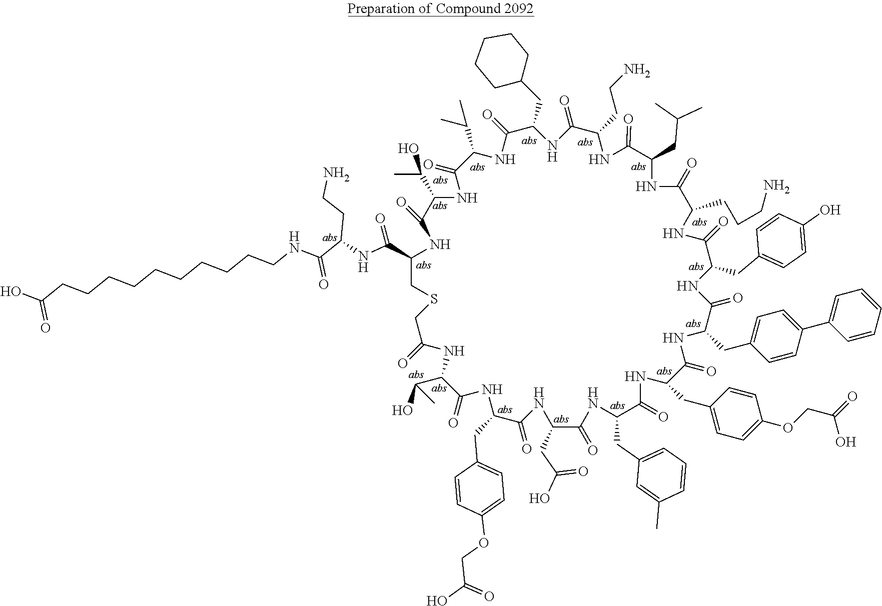

Compound 2092 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20.3 mg, and its estimated purity by LCMS analysis was 98.1%. Analysis condition 2: Retention time=1.52 min; ESI-MS(+) m/z [M+3H]³⁺: 778.1.

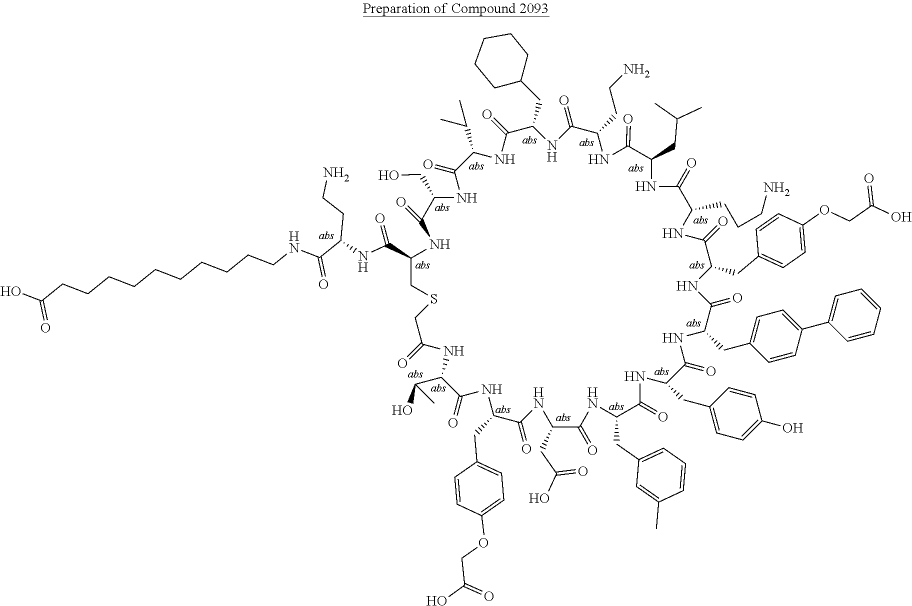

Compound 2093 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 2.2 mg, and its estimated purity by LCMS analysis was 97%. Analysis condition 1: Retention time=1.59 min; ESI-MS(+) m/z [M+2H]²⁺: 1160.2.

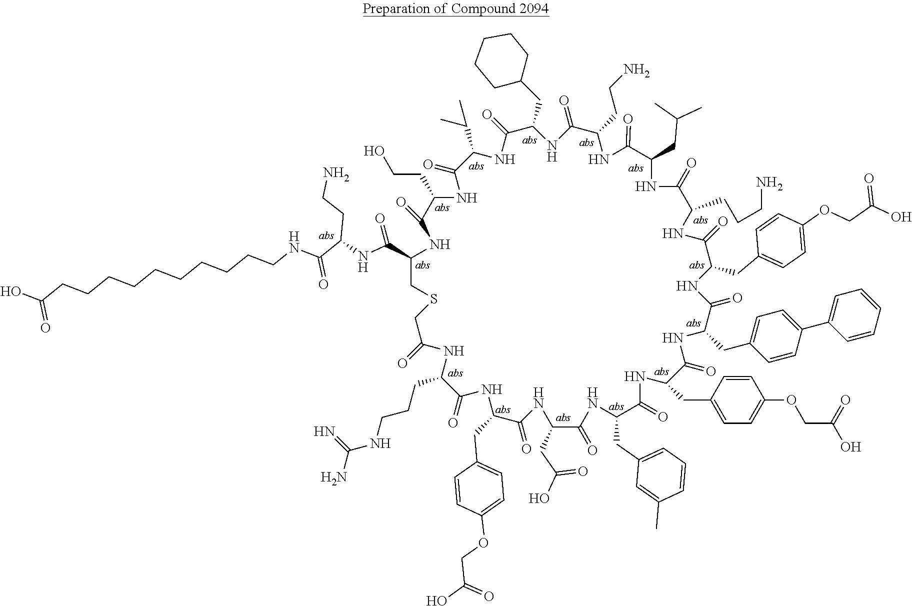

Compound 2094 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 3.1 mg, and its estimated purity by LCMS analysis was 96.1%. Analysis condition 2: Retention time=1.4 min; ESI-MS(+) m/z [M+2H]²⁺: 1223.4.

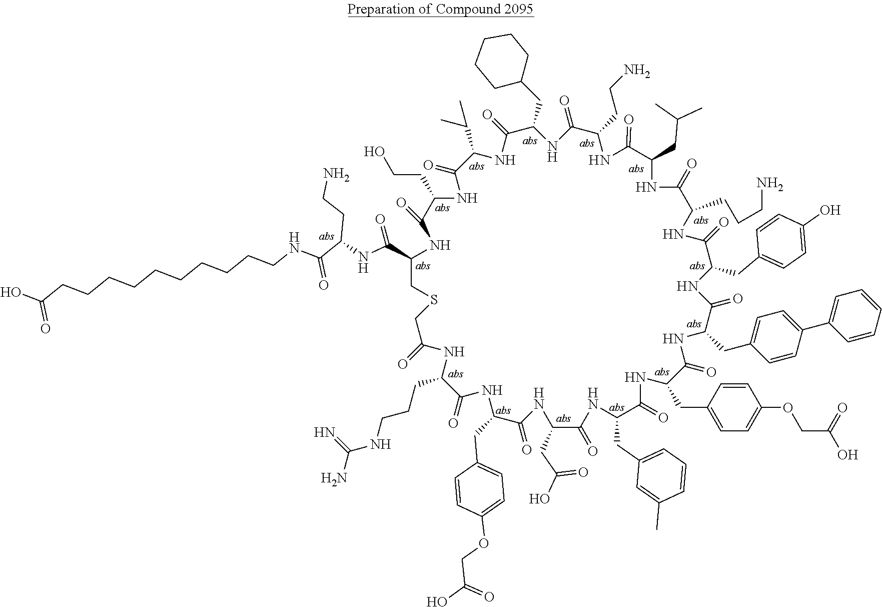

Compound 2095 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 6.4 mg, and its estimated purity by LCMS analysis was 96.8%. Analysis condition 1: Retention time=1.57 min; ESI-MS(+) m/z [M+2H]²⁺: 1194.

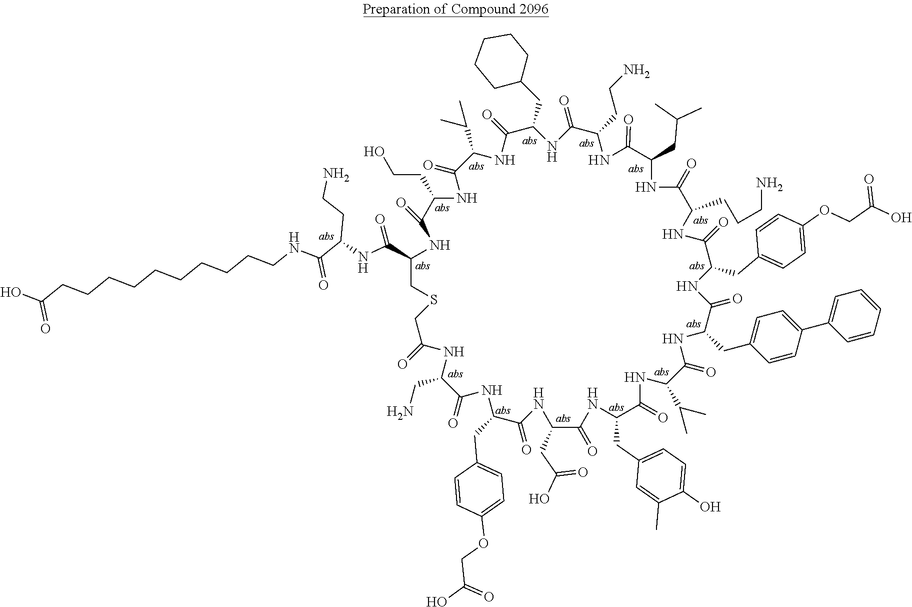

Compound 2096 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.9 mg, and its estimated purity by LCMS analysis was 81.4%. Analysis condition 1: Retention time=1.51 min; ESI-MS(+) m/z [M+2H]²⁺: 1134.

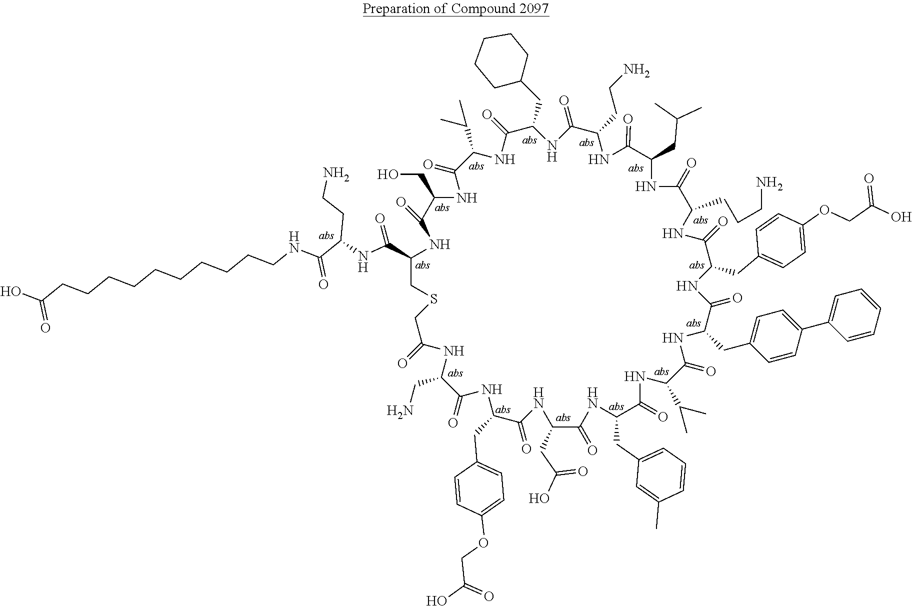

Compound 2097 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 18.3 mg, and its estimated purity by LCMS analysis was 82.7%. Analysis condition 1: Retention time=1.55 min; ESI-MS(+) m/z [M+2H]²⁺: 1120.

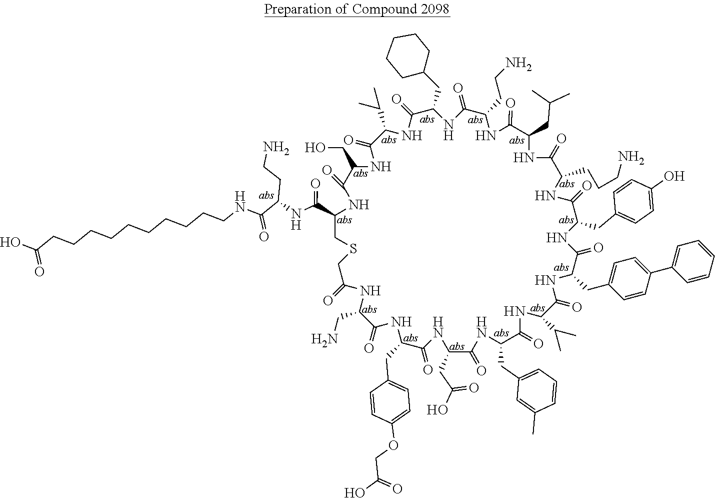

Compound 2098 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.9 mg, and its estimated purity by LCMS analysis was 80.2%. Analysis condition 2: Retention time=1.64 min; ESI-MS(+) m/z [M+2H]²⁺: 1091.1.

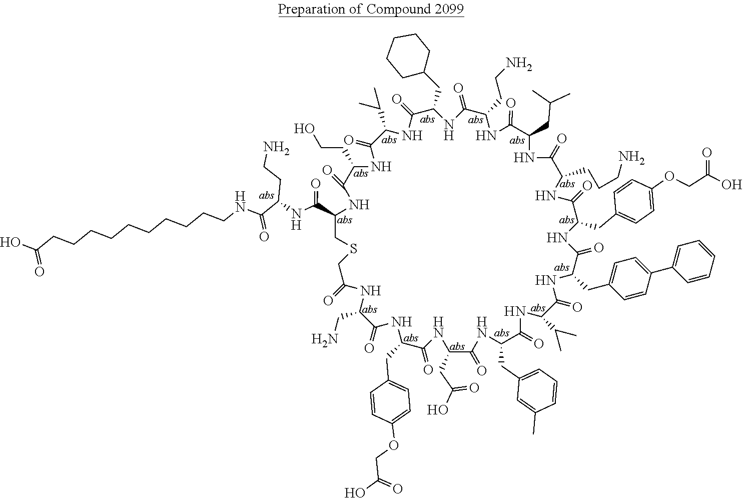 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 2099 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.7 mg, and its estimated purity by LCMS analysis was 97.2%. Analysis condition 1: Retention time=1.63 min; ESI-MS(+) m/z [M+2H]²⁺: 1127.

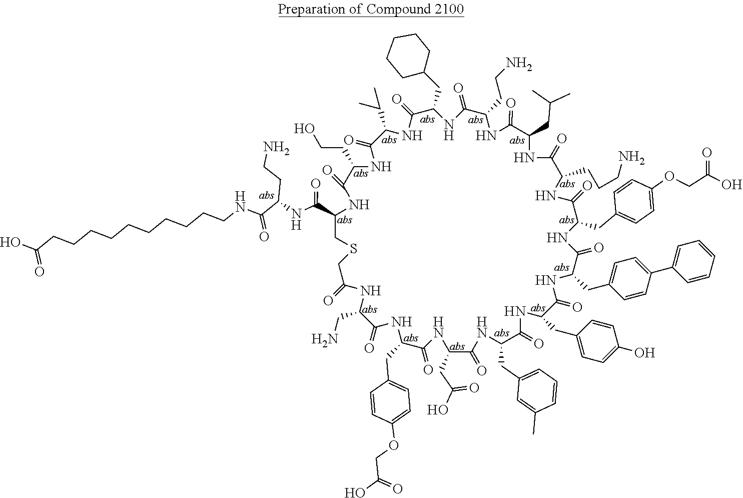 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2100 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.7 mg, and its estimated purity by LCMS analysis was 95.5%. Analysis condition 2: Retention time=1.61 min; ESI-MS(+) m/z [M+2H]²⁺: 1159.2.

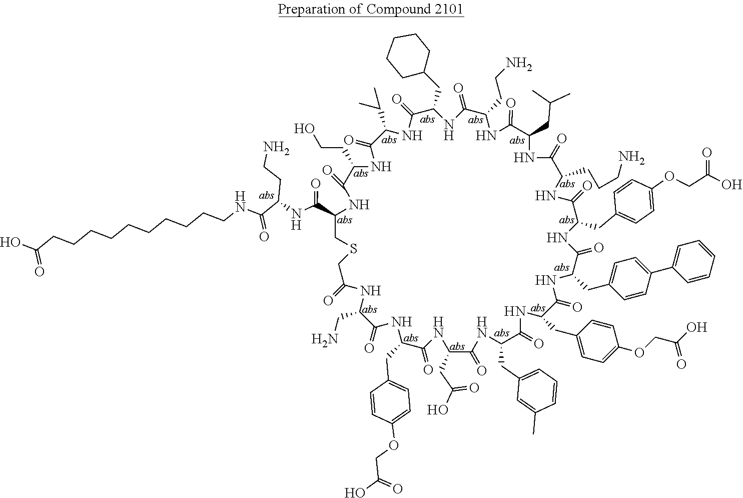 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2101 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.7 mg, and its estimated purity by LCMS analysis was 100%. Analysis condition 2: Retention time=1.58 min; ESI-MS(+) m/z [M+2H]²⁺: 1188.

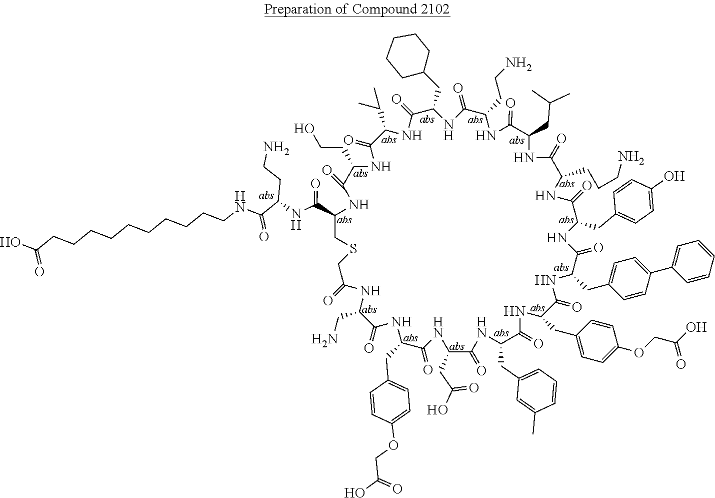 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Compound 2102 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 3.9 mg, and its estimated purity by LCMS analysis was 96.8%. Analysis condition 1: Retention time=1.57 min; ESI-MS(+) m/z [M+2H]²⁺: 1159.

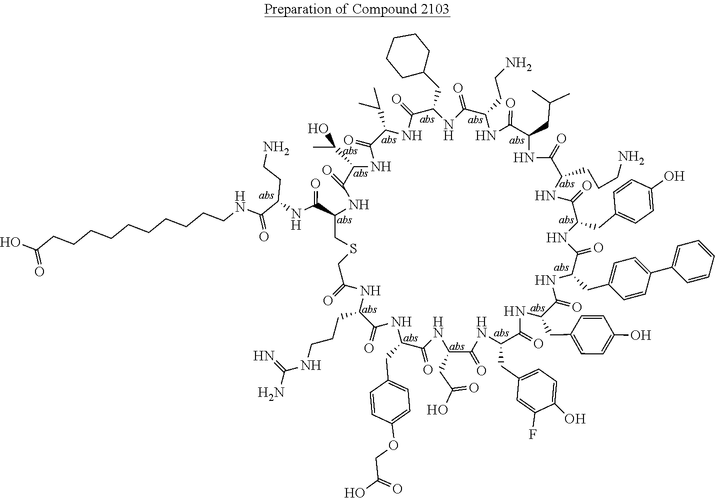 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2ccc(O)c(F)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2103 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 4.9 mg, and its estimated purity by LCMS analysis was 100%. Analysis condition 2: Retention time=1.51 min; ESI-MS(+) m/z [M+2H]²⁺: 1175.1.

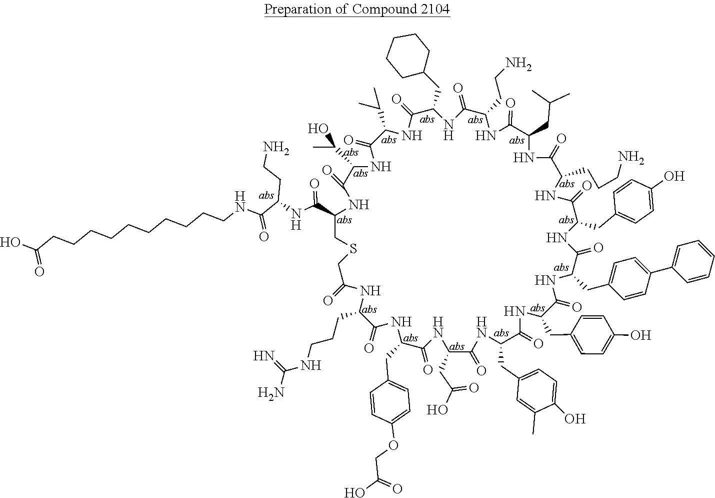 [SMILES: Cc1cc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)ccc1O]

Compound 2104 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.1 mg, and its estimated purity by LCMS analysis was 100% as a mixture of diastereomers. Analysis condition 2: Retention time=1.48, 1.51 min; ESI-MS(+) m/z [M+2H]²⁺: 1172.79, 1172.79.

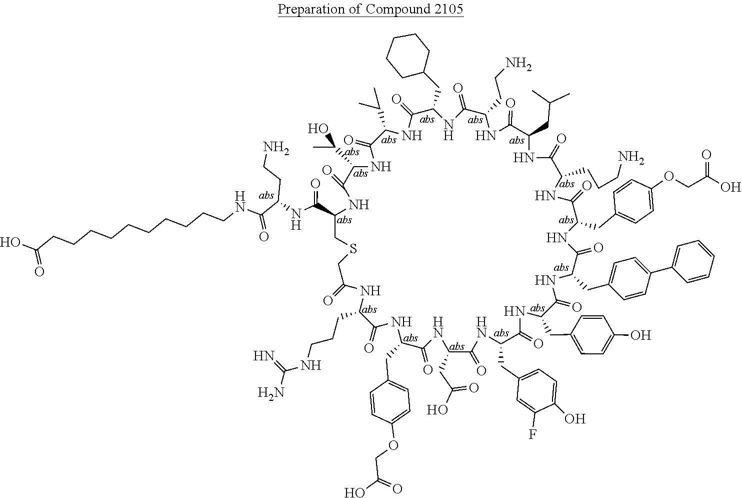 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(O)cc2)NC(=O)[C@H](Cc2ccc(O)c(F)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2105 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 9.1 mg, and its estimated purity by LCMS analysis was 80.6%. Analysis condition 1: Retention time=1.51 min; ESI-MS(+) m/z [M+2H]²⁺: 1204.1.

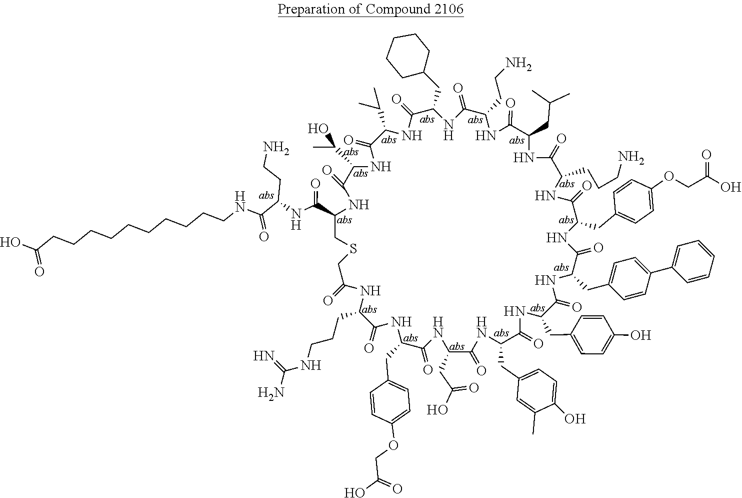 [SMILES: Cc1cc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)ccc1O]

Compound 2106 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.5 mg, and its estimated purity by LCMS analysis was 100%. Analysis condition 2: Retention time=1.48 min; ESI-MS(+) m/z [M+2H]²⁺: 1202.1.

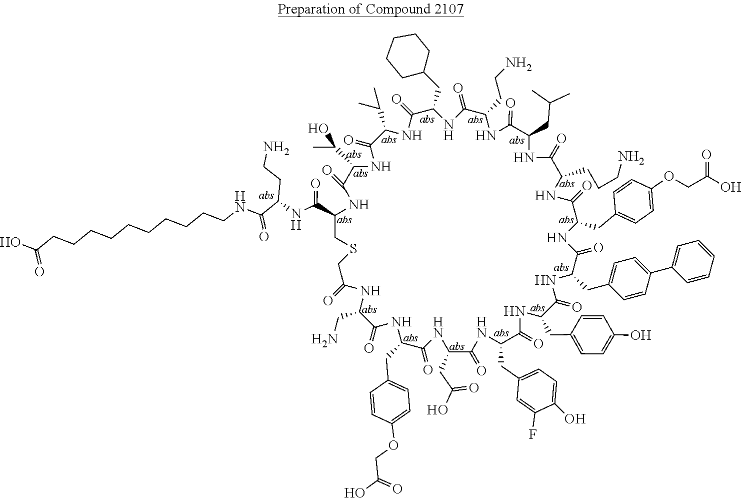 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(O)cc2)NC(=O)[C@H](Cc2ccc(O)c(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2107 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10 mg, and its estimated purity by LCMS analysis was 80% as a mixture of diastereomers. Analysis condition 2: Retention time=1.46, 1.50 min; ESI-MS(+) m/z [M+2H]²⁺: 1168.88, 1169.06.

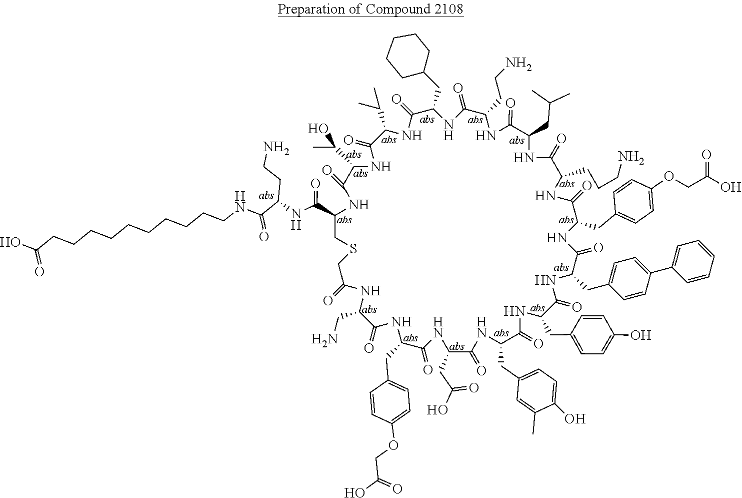 [SMILES: Cc1cc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)ccc1O]

Compound 2108 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Tyr(3-Me)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 3.3 mg, and its estimated purity by LCMS analysis was 95.7%. Analysis condition 2: Retention time=1.5 min; ESI-MS(+) m/z [M+2H]²⁺: 1167.2.

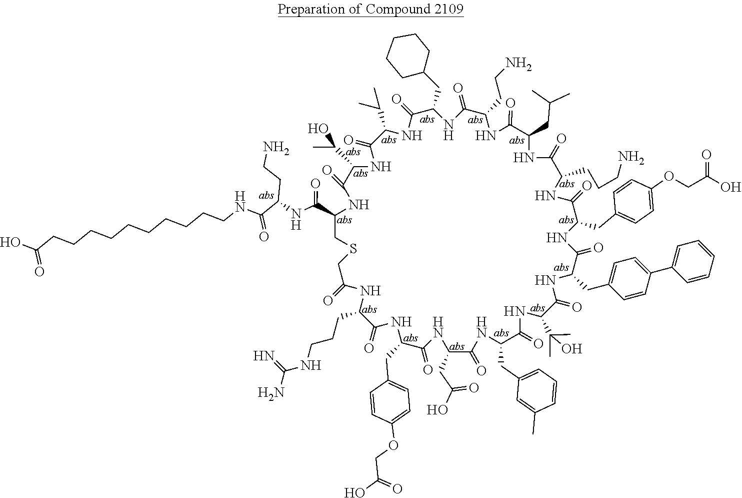 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)O)NC2=O)c1]

Compound 2109 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6 mg, and its estimated purity by LCMS analysis was 91%. Analysis condition 1: Retention time=1.6 min; ESI-MS(+) m/z [M+2H]²⁺: 1170.2.

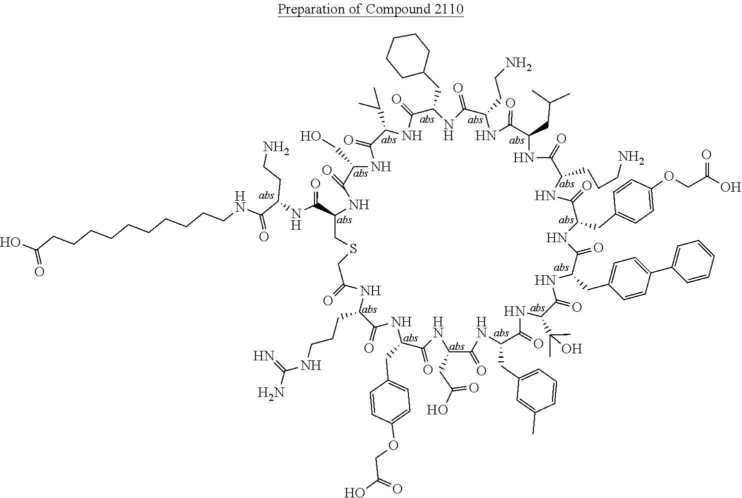 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)O)NC2=O)c1]

Compound 2110 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 26.7 mg, and its estimated purity by LCMS analysis was 93.1%. Analysis condition 2: Retention time=1.47 min; ESI-MS(+) m/z [M+2H]²⁺: 1163.5.

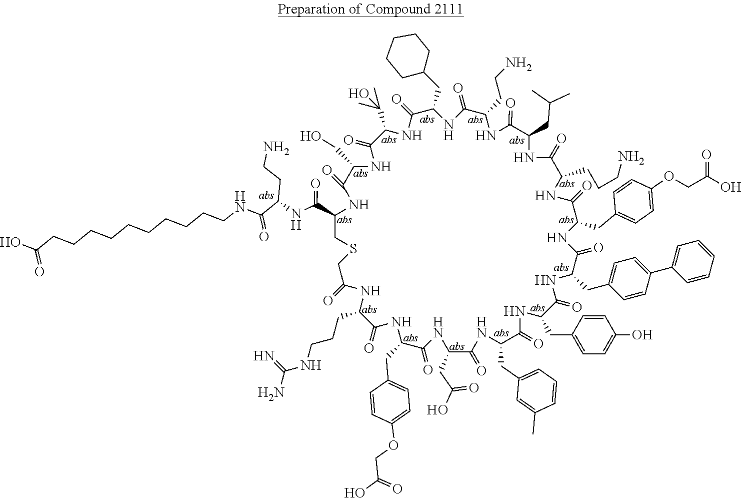 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2111 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 80.6 mg, and its estimated purity by LCMS analysis was 95.8%. Analysis condition 1: Retention time=1.6 min; ESI-MS(+) m/z [M+2H]²⁺: 1195.3.

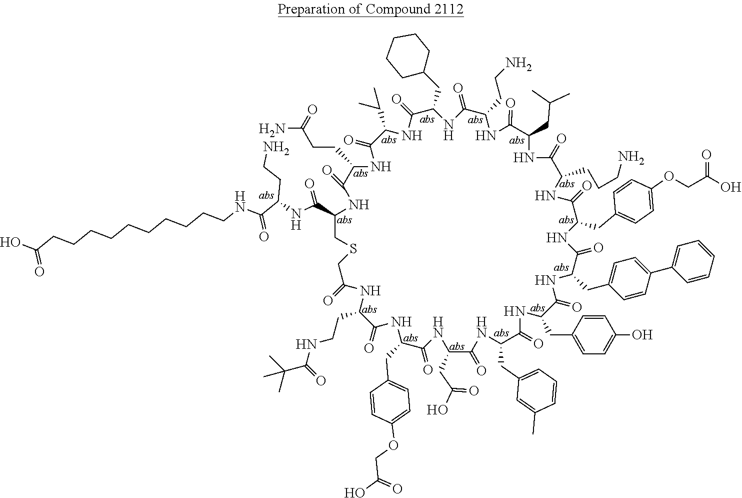 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)C(C)(C)C)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2112 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Dab(COtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 60.4 mg, and its estimated purity by LCMS analysis was 93.6%. Analysis condition 1: Retention time=1.73 min; ESI-MS(+) m/z [M+3H]³⁺: 814.9.

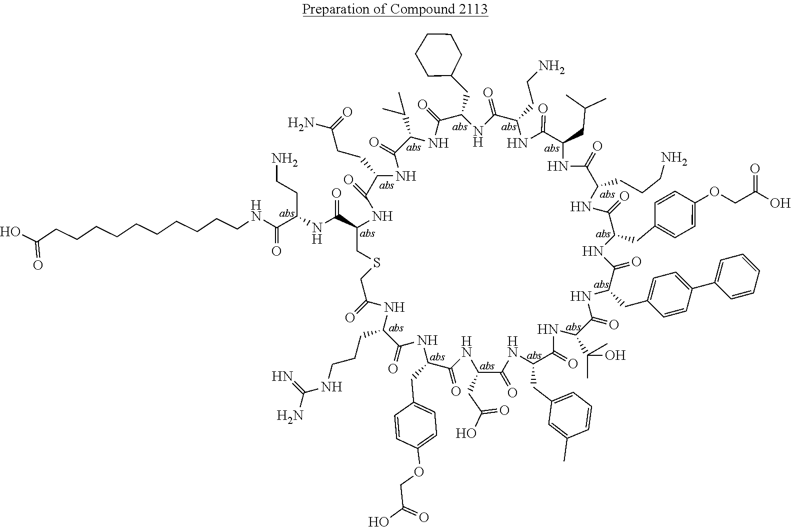 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)O)NC2=O)c1]

Compound 2113 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.1 mg, and its estimated purity by LCMS analysis was 95%. Analysis condition 1: Retention time=1.57 min; ESI-MS(+) m/z [M+3H]³⁺: 789.2.

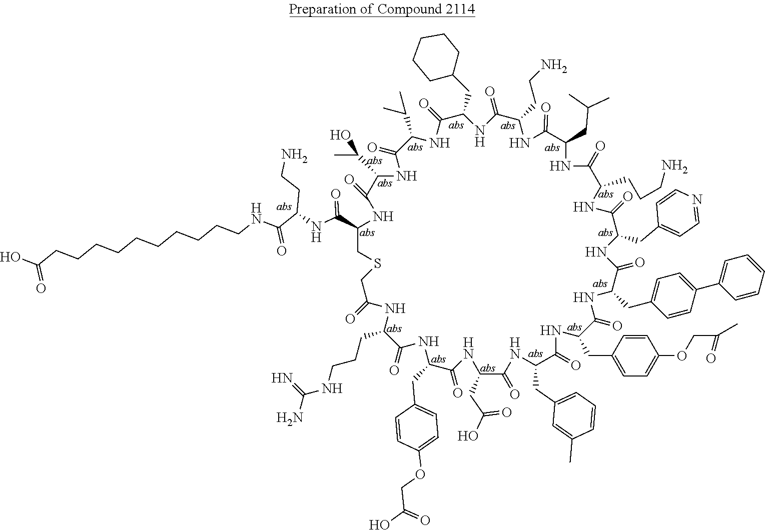 [SMILES: CC(=O)COc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccncc3)NC(=O)[C@H](Cc3ccc(-c4ccccc4)cc3)NC2=O)cc1]

Compound 2114 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by UV signals. Fractions containing the desired product were combined and dried via centrifugal evaporation. The yield of the product was 26.3 mg, and its estimated purity by LCMS analysis was 86.4%. Analysis condition 1: Retention time=1.51 min; ESI-MS(+) m/z [M+2H]²⁺: 1184.4.

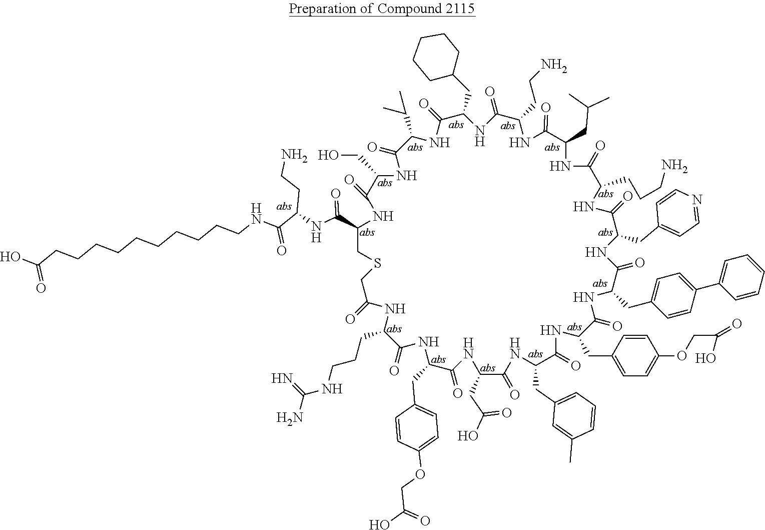 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccncc3)NC(=O)[C@H](Cc3ccc(-c4ccccc4)cc3)NC(=O)[C@H](Cc3ccc(OCC(=O)O)cc3)NC2=O)c1]

Compound 2115 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 1.3 mg, and its estimated purity by LCMS analysis was 100%. Analysis condition 2: Retention time=1.51 min; ESI-MS(+) m/z [M+3H]³⁺: 786.4.

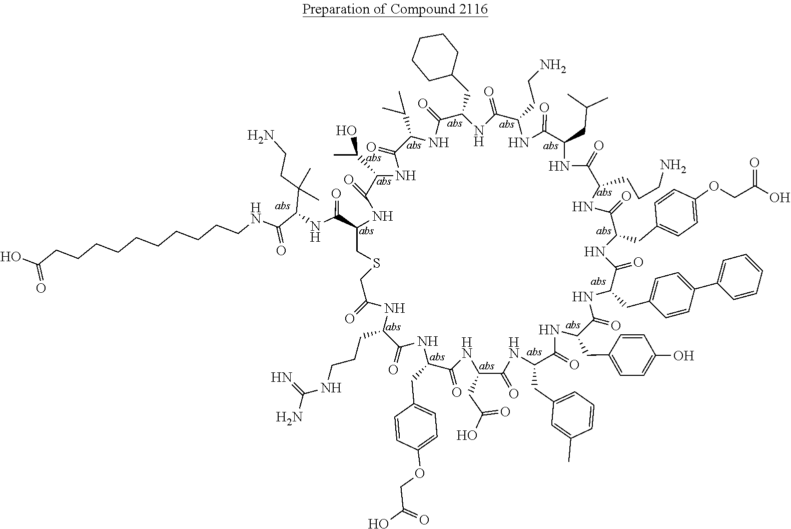 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](C(=O)NCCCCCCCCCCC(=O)O)C(C)(C)CCN)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2116 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with(S)-2-((((9H-fluoren-9-yl) methoxy) carbonyl)amino)-5-((tert-butoxycarbonyl)amino)-3,3-dimethylpentanoic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.1 mg, and its estimated purity by LCMS analysis was 100%. Analysis condition 2: Retention time=1.65 min; ESI-MS(+) m/z [M+2H]²⁺: 1215.1.

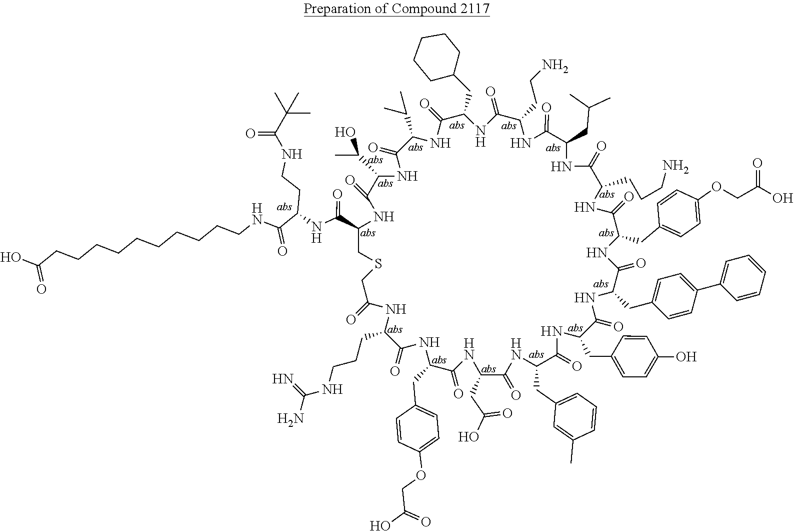 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCNC(=O)C(C)(C)C)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2117 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Dab(COtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14 mg, and its estimated purity by LCMS analysis was 86.5%. Analysis condition 1: Retention time=1.75 min; ESI-MS(+) m/z [M+2H]²⁺: 1236.3.

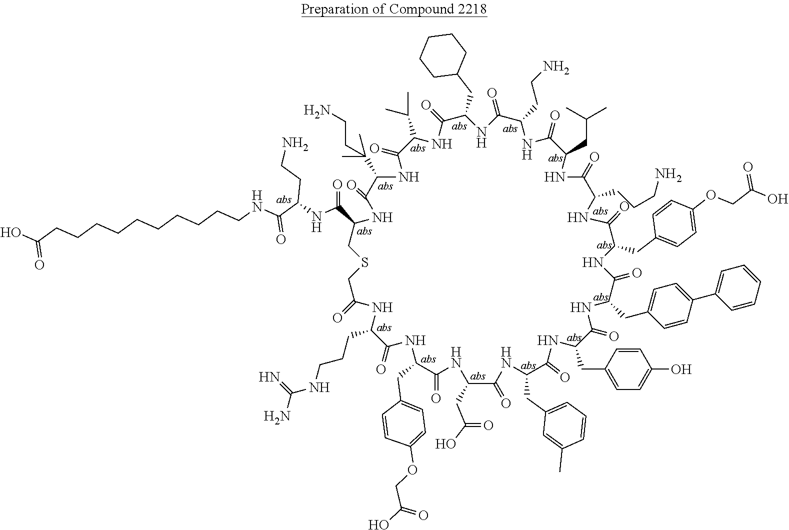

Compound 2118 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with(S)-2-((((9H-fluoren-9-yl) methoxy) carbonyl)amino)-5-((tert-butoxycarbonyl)amino)-3,3-dimethylpentanoic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 7.6 mg, and its estimated purity by LCMS analysis was 95.3%. Analysis condition 2: Retention time=1.55 min; ESI-MS(+) m/z [M+2H]²⁺: 1214.3.

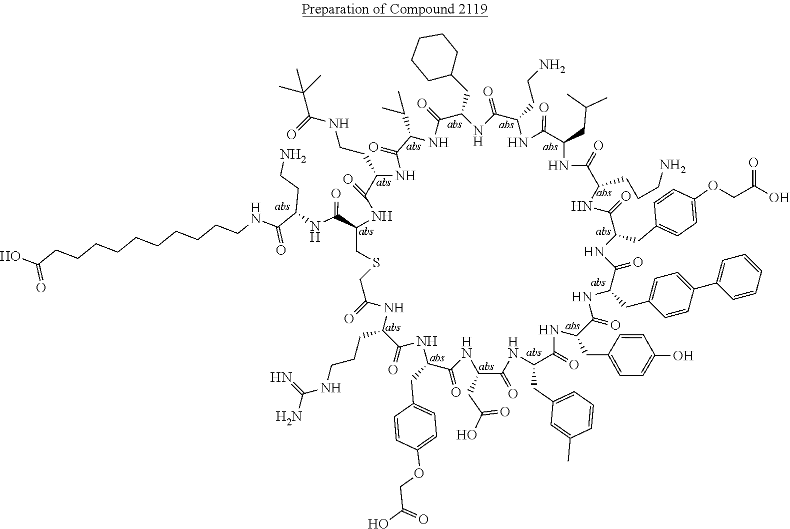 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCNC(=O)C(C)(C)C)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2119 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Dab(COtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 35 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.2 mg, and its estimated purity by LCMS analysis was 95.1%. Analysis condition 2: Retention time=1.64 min; ESI-MS(+) m/z [M+2H]²⁺: 1235.3.

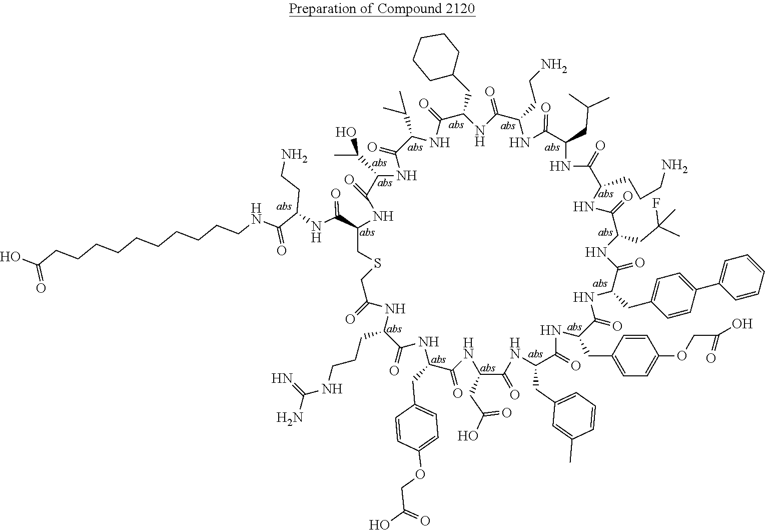 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CC(C)(C)F)NC(=O)[C@H](Cc3ccc(-c4ccccc4)cc3)NC(=O)[C@H](Cc3ccc(OCC(=O)O)cc3)NC2=O)c1]

Compound 2120 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 0.8 mg, and its estimated purity by LCMS analysis was 100%. Analysis condition 2: Retention time=1.5 min; ESI-MS(+) m/z [M+3H]³⁺: 781.2.

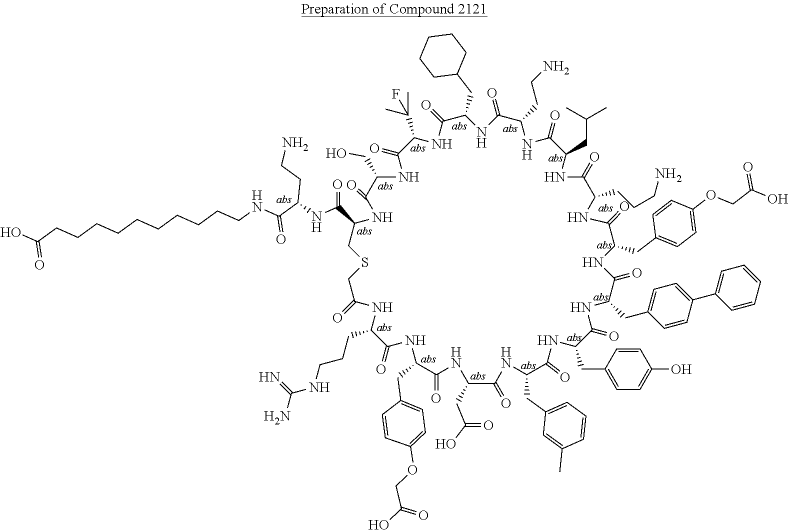 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)(C)F)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2121 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.9 mg, and its estimated purity by LCMS analysis was 98.4%. Analysis condition 1: Retention time=1.5 min; ESI-MS(+) m/z [M+2H]²⁺: 1196.

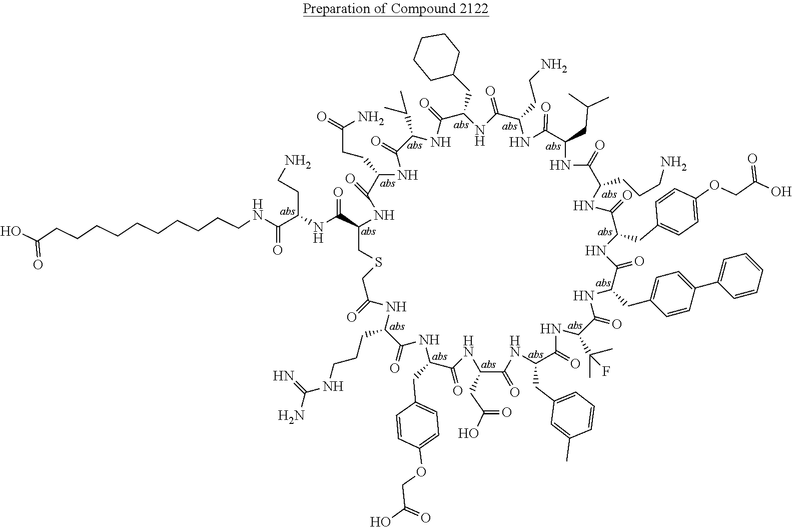 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)F)NC2=O)c1]

Compound 2122 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.9 mg, and its estimated purity by LCMS analysis was 94.9%. Analysis condition 1: Retention time=1.58 min; ESI-MS(+) m/z [M+3H]³⁺: 790.1.

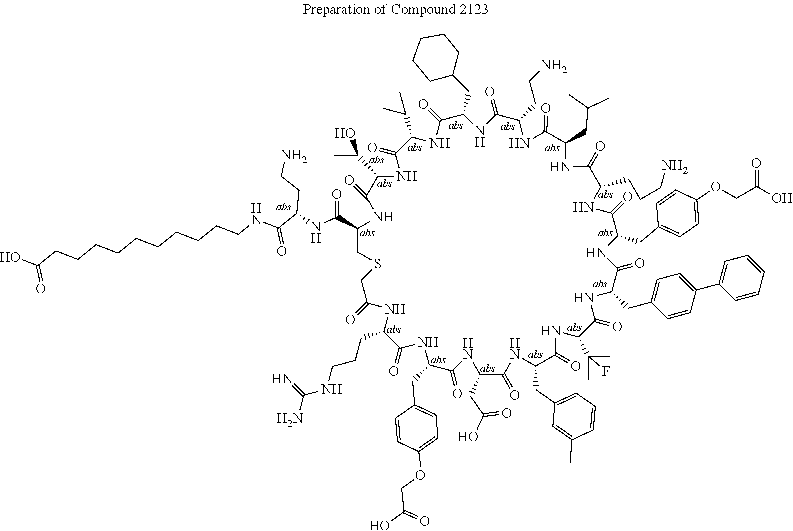 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)F)NC2=O)c1]

Compound 2123 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.7 mg, and its estimated purity by LCMS analysis was 93.9%. Analysis condition 1: Retention time=1.58 min; ESI-MS(+) m/z [M+2H]²⁺: 1171.2.

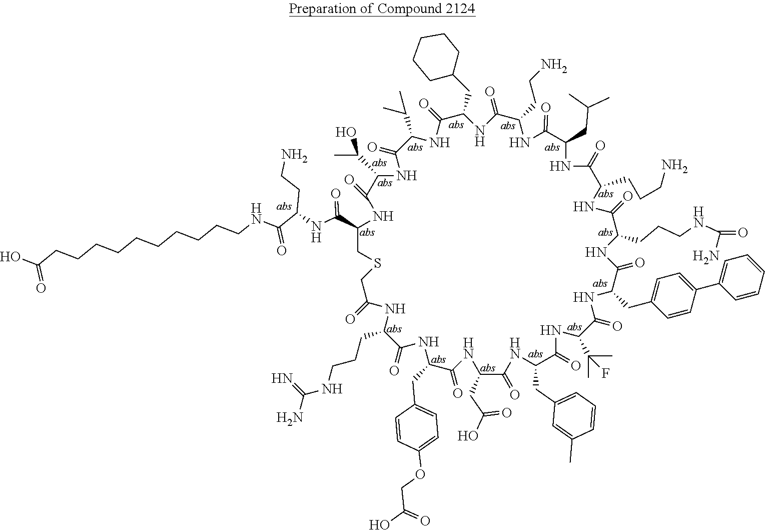 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)(C)F)NC2=O)c1]

Compound 2124 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.5 mg, and its estimated purity by LCMS analysis was 93.1%. Analysis condition 1: Retention time=1.57 min; ESI-MS(+) m/z [M+2H]²⁺: 1138.8.

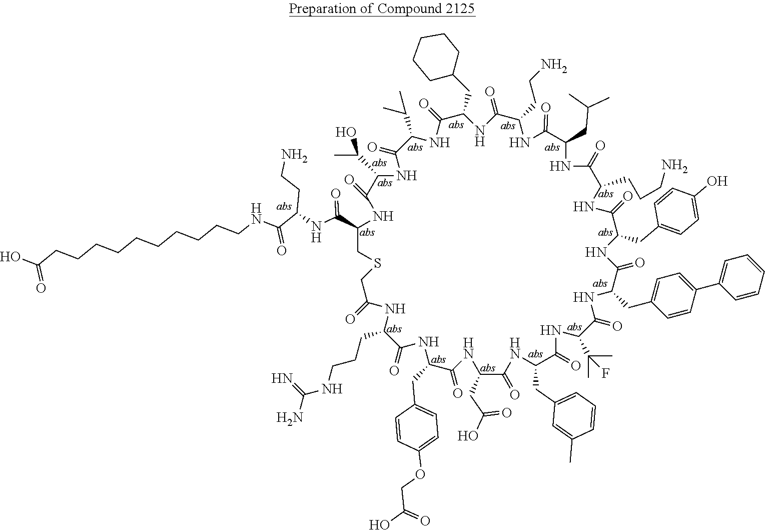 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)(C)F)NC2=O)c1]

Compound 2125 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 38.8 mg, and its estimated purity by LCMS analysis was 96.5%. Analysis condition 1: Retention time=1.58 min; ESI-MS(+) m/z [M+2H]²⁺: 1142.

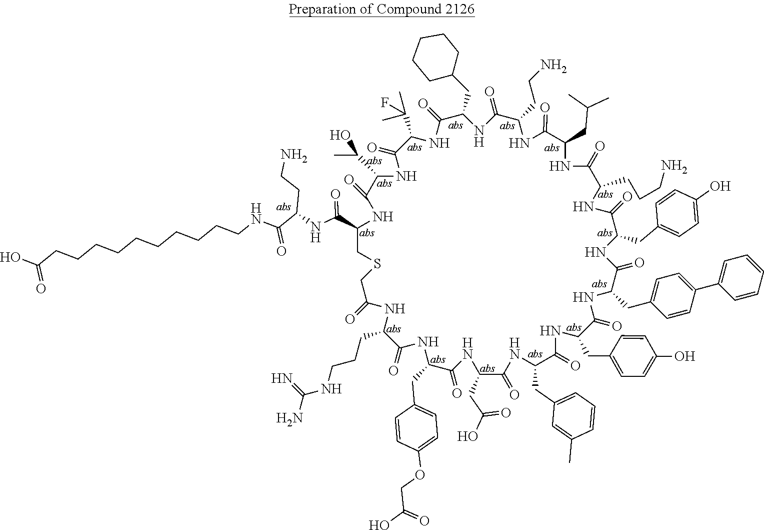 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)(C)F)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2126 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 49.4 mg, and its estimated purity by LCMS analysis was 97%. Analysis condition 2: Retention time=1.57 min; ESI-MS(+) m/z [M+2H]²⁺: 1173.9.

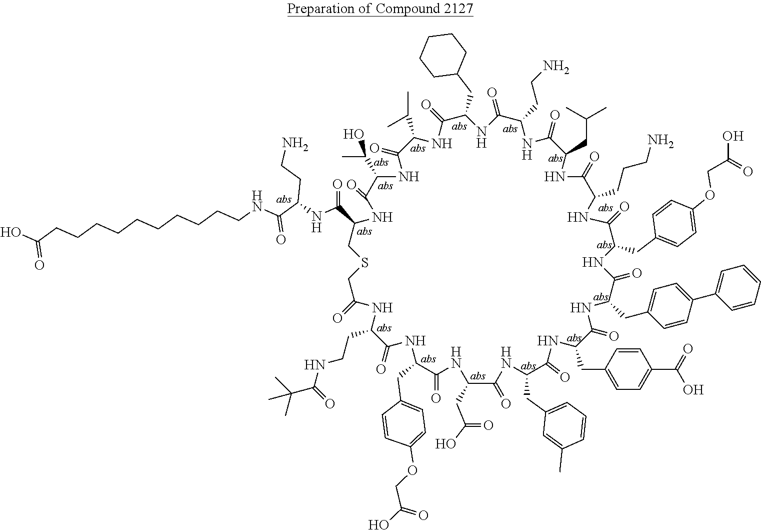 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)C(C)(C)C)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2127 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Dab(COtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.3 mg, and its estimated purity by LCMS analysis was 100%. Analysis condition 1: Retention time=1.87 min; ESI-MS(+) m/z [M+2H]²⁺: 1222.

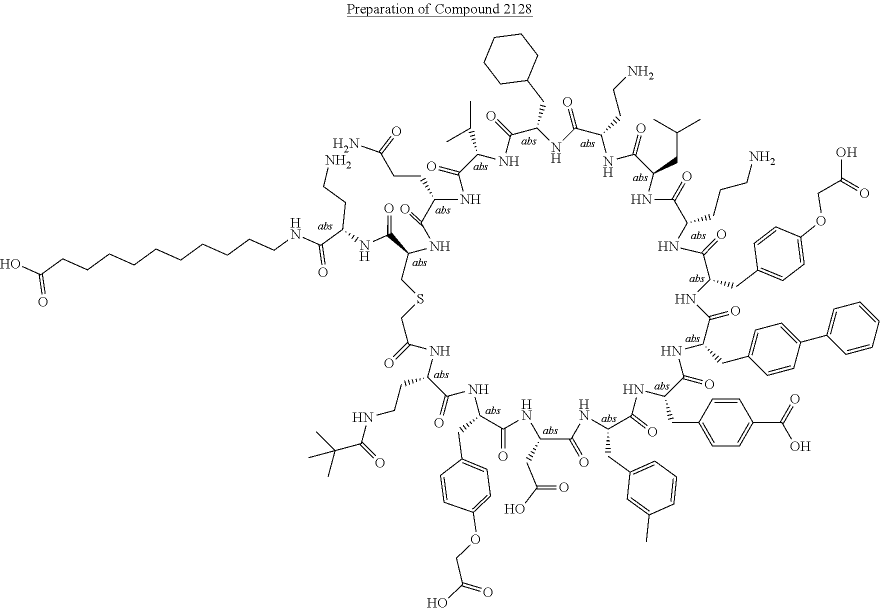 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)C(C)(C)C)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2128 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure Fmoc-Dab(COtBu)-OH”, “Symphony X Single-Coupling Manual Addition Procedure A” was followed with; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 42.9 mg, and its estimated purity by LCMS analysis was 96.2%. Analysis condition 2: Retention time=1.46 min; ESI-MS(+) m/z [M+2H]²⁺: 1236.

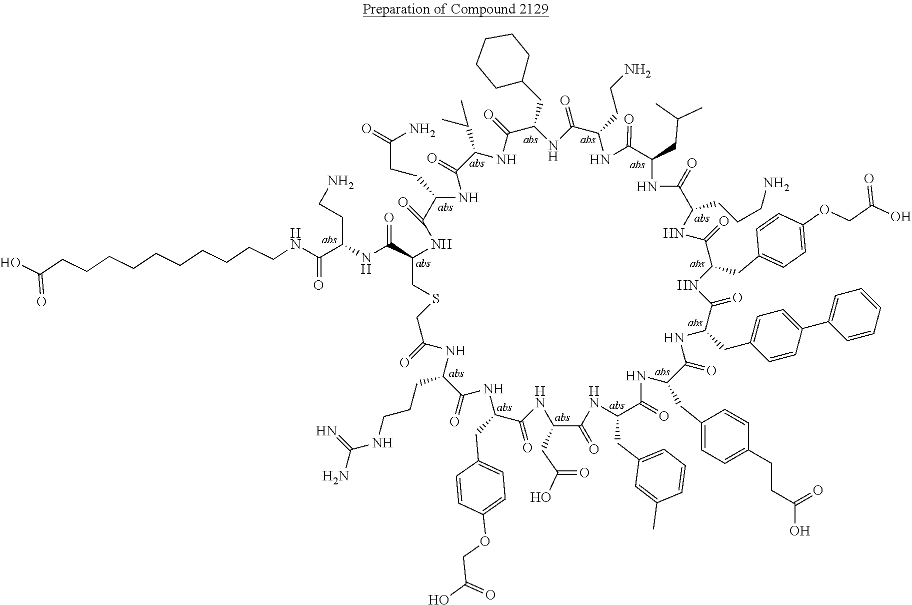 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(CCC(=O)O)cc3)NC2=O)c1]

Compound 2129 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8 mg, and its estimated purity by LCMS analysis was 90%. Analysis condition 1: Retention time=1.58 min; ESI-MS(+) m/z [M+2H]²⁺: 1237.4.

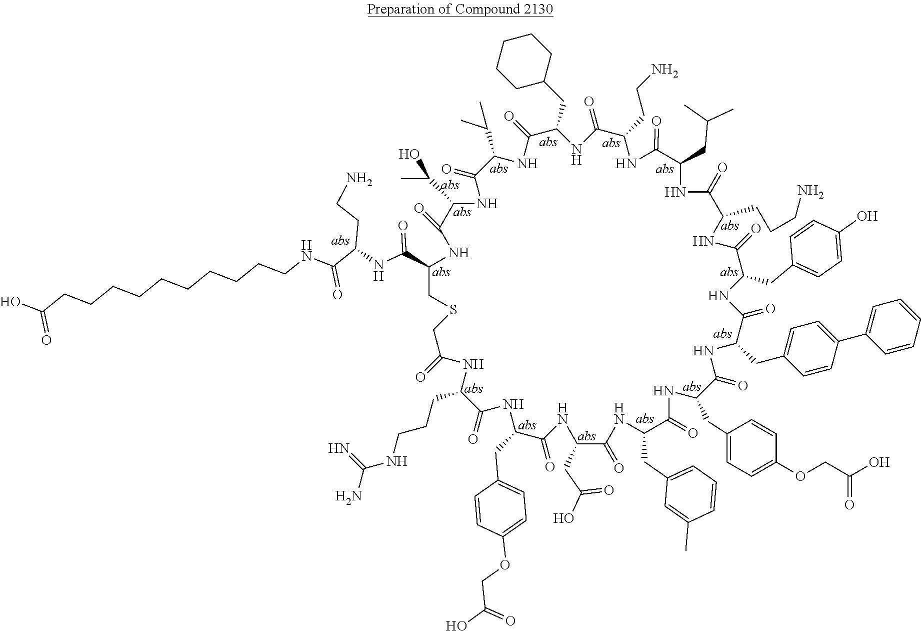 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Compound 2130 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 9.6 mg, and its estimated purity by LCMS analysis was 90.2%. Analysis condition: Retention time=1.69 min; ESI-MS(+) m/z [M+2H]²⁺: 1194.2.

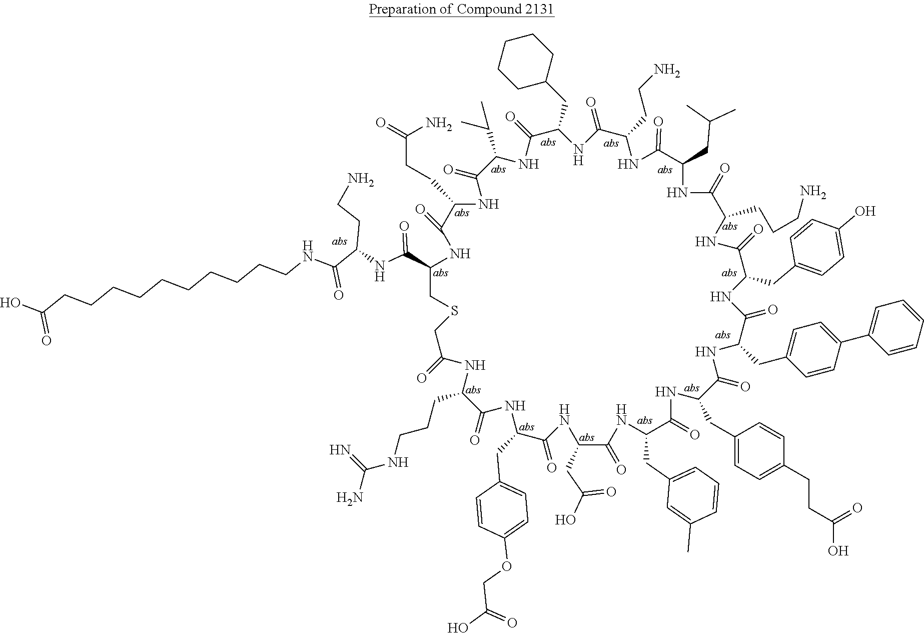 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(CCC(=O)O)cc3)NC2=O)c1]

Compound 2131 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16 mg, and its estimated purity by LCMS analysis was 98.1%. Analysis condition 2: Retention time=1.48 min; ESI-MS(+) m/z [M+2H]²⁺: 1207.3.

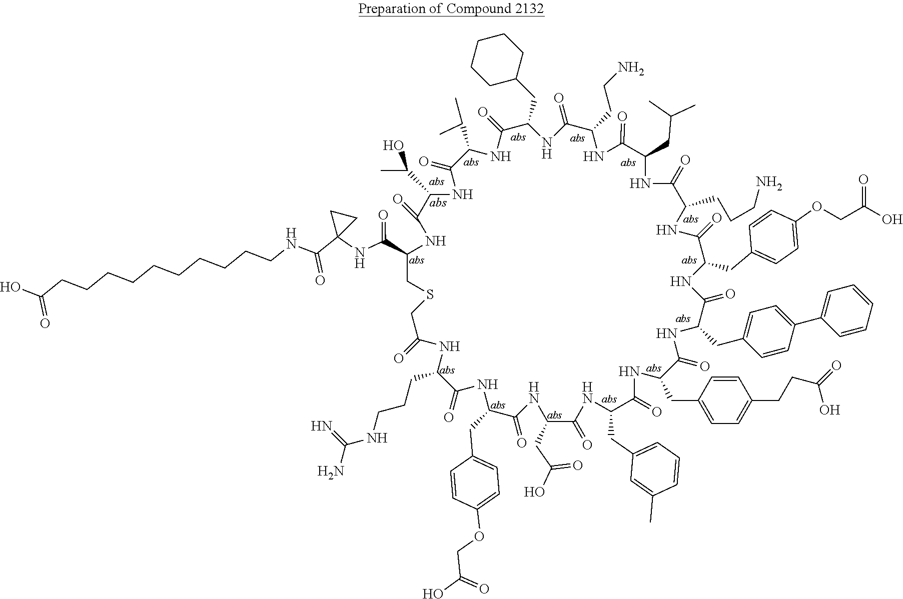 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NC3(C(=O)NCCCCCCCCCCC(=O)O)CC3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(CCC(=O)O)cc3)NC2=O)c1]

Compound 2132 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 30.3 mg, and its estimated purity by LCMS analysis was 97%. Analysis condition 1: Retention time=1.68 min; ESI-MS(+) m/z [M+2H]²⁺: 810.1.

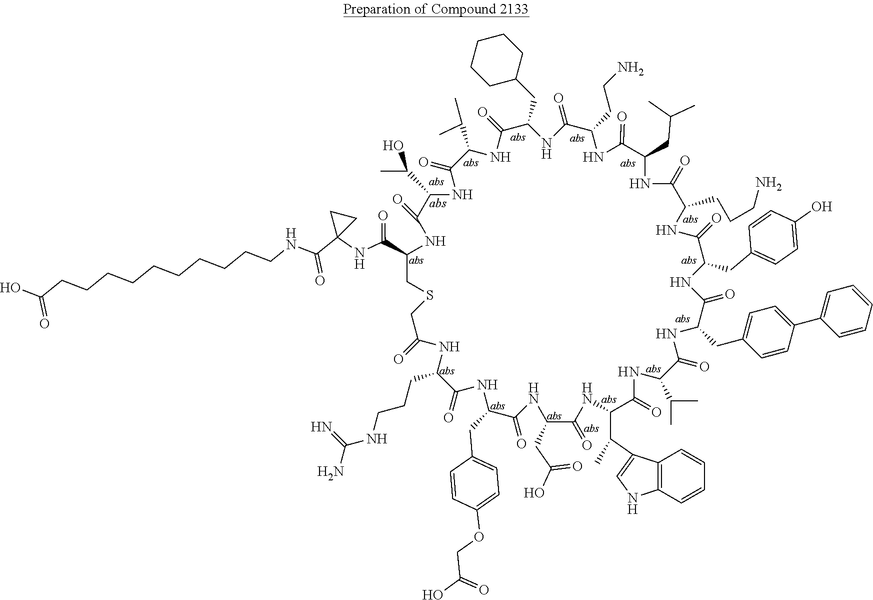

Compound 2133 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 35.5 mg, and its estimated purity by LCMS analysis was 97%. Analysis condition 1: Retention time=1.63 min; ESI-MS(+) m/z [M+2H]²⁺: 1144.1.

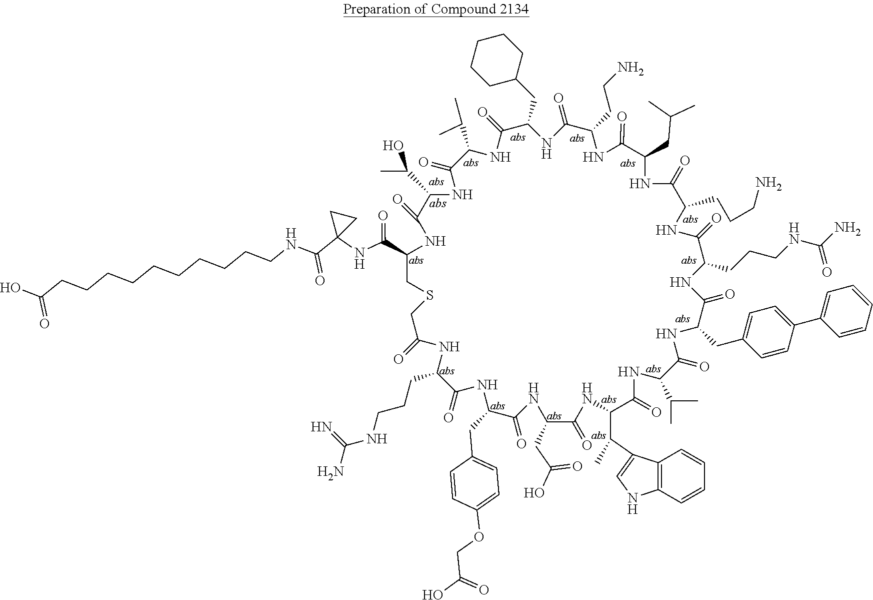 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H]([C@@H](C)c2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NC2(C(=O)NCCCCCCCCCCC(=O)O)CC2)NC(=O)[C@H]([C@@H](C)O)NC(=O)[C@H](C(C)C)NC(=O)[C@H](CC2CCCCC2)NC(=O)[C@H](CCN)NC1=O]

Compound 2134 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.1 mg, and its estimated purity by LCMS analysis was 97%. Analysis condition: Retention time=1.68 min; ESI-MS(+) m/z [M+2H]²⁺: 1141.1.

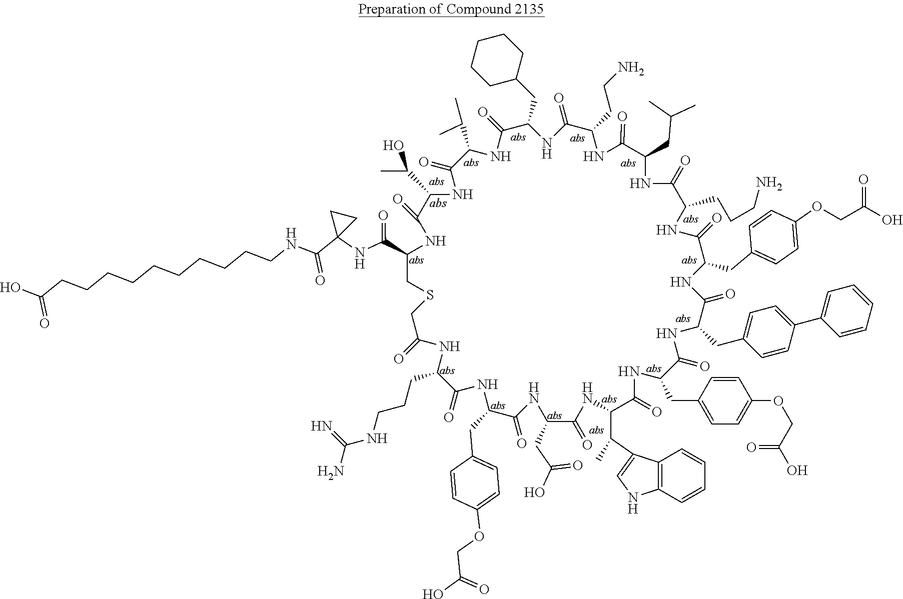 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H]([C@@H](C)c2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NC2(C(=O)NCCCCCCCCCCC(=O)O)CC2)NC(=O)[C@H]([C@@H](C)O)NC(=O)[C@H](C(C)C)NC(=O)[C@H](CC2CCCCC2)NC(=O)[C@H](CCN)NC1=O]

Compound 2135 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 30.2 mg, and its estimated purity by LCMS analysis was 96.2%. Analysis condition 1: Retention time=1.63 min; ESI-MS(+) m/z [M+2H]²⁺: 1234.1.

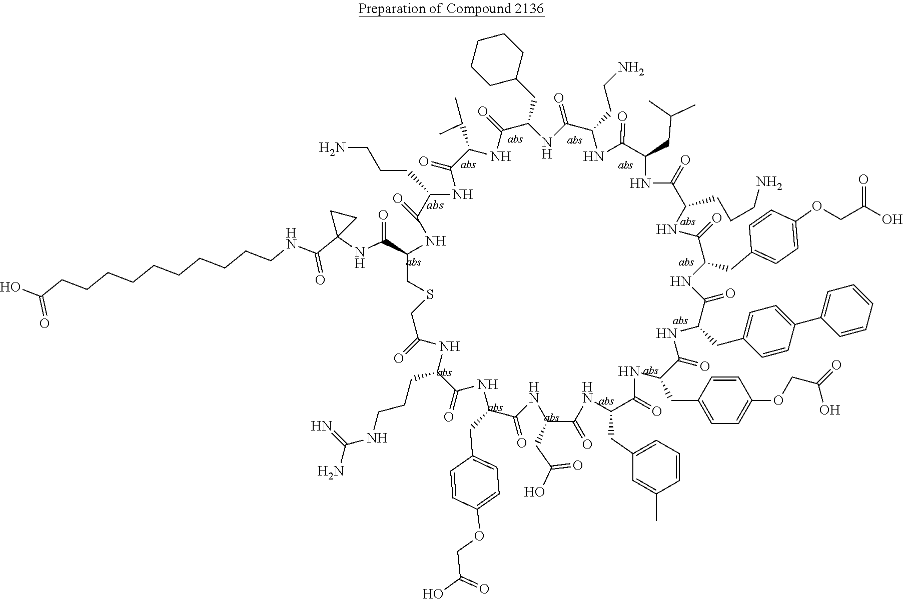 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NC3(C(=O)NCCCCCCCCCCC(=O)O)CC3)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2136 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 26.1 mg, and its estimated purity by LCMS analysis was 95.3%. Analysis condition 1: Retention time=1.43 min; ESI-MS(+) m/z [M+3H]³⁺: 814.1.

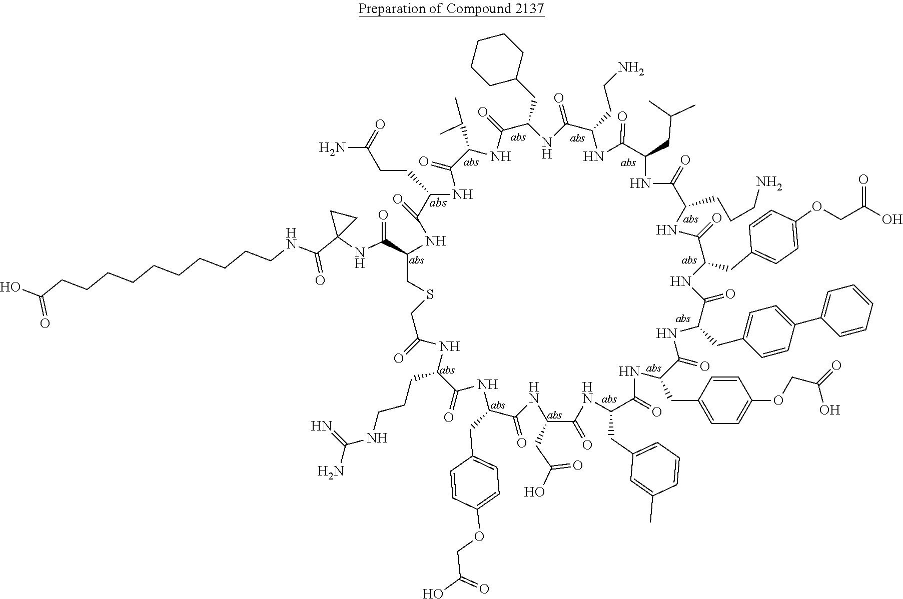 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NC3(C(=O)NCCCCCCCCCCC(=O)O)CC3)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2137 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 25 mg, and its estimated purity by LCMS analysis was 100%. Analysis condition 2: Retention time=1.32 min; ESI-MS(+) m/z [M+2H]²⁺: 1228.2.

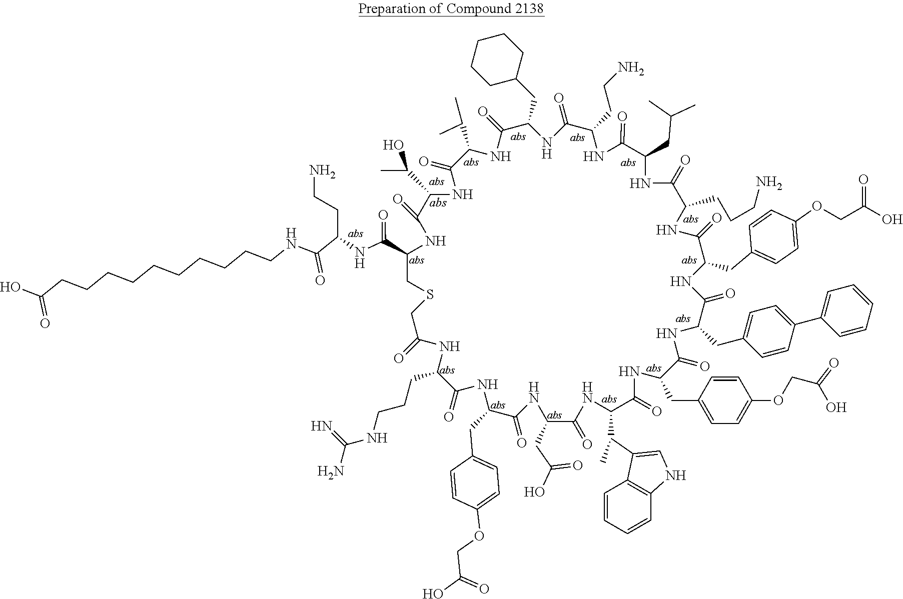 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H]([C@@H](C)c2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2138 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4 mg, and its estimated purity by LCMS analysis was 91.9%. Analysis condition: Retention time=1.57 min; ESI-MS(+) m/z [M+3H]³⁺: 828.5.

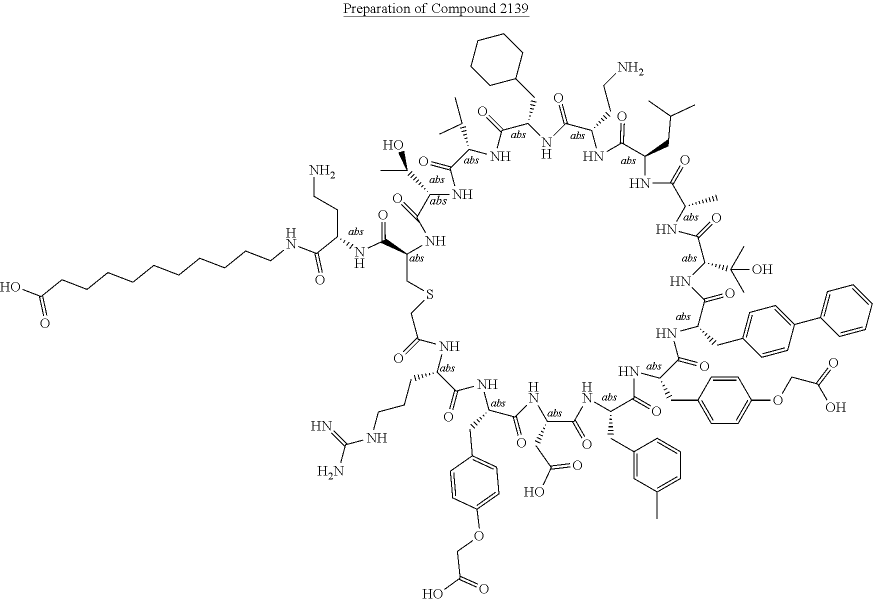 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)NC(=O)[C@H](C(C)(C)O)NC(=O)[C@H](Cc3ccc(-c4ccccc4)cc3)NC(=O)[C@H](Cc3ccc(OCC(=O)O)cc3)NC2=O)c1]

Compound 2139 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 6 mg, and its estimated purity by LCMS analysis was 99.1%. Analysis condition 1: Retention time=1.6 min; ESI-MS(+) m/z [M+2H]²⁺: 1170.1.

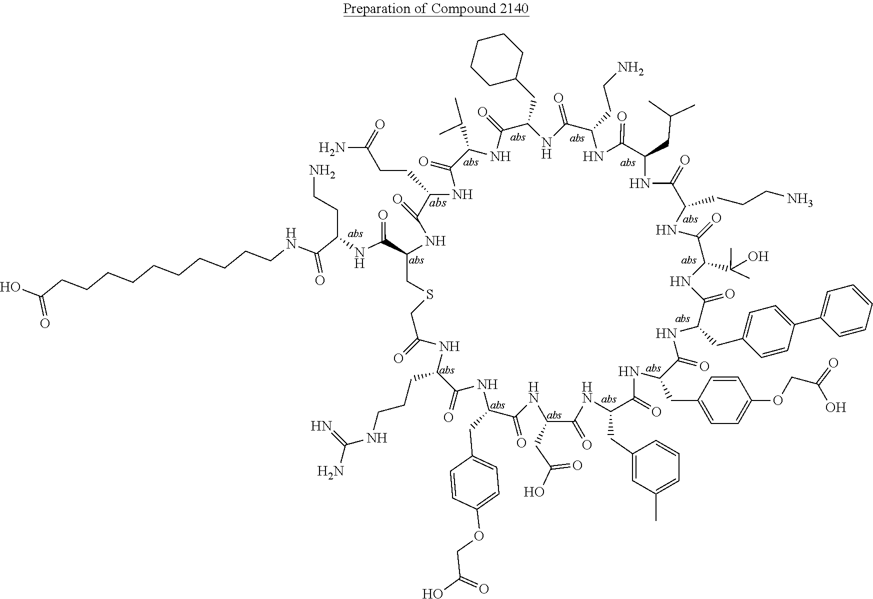 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)(C)O)NC(=O)[C@H](Cc3ccc(-c4ccccc4)cc3)NC(=O)[C@H](Cc3ccc(OCC(=O)O)cc3)NC2=O)c1]

Compound 2140 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 13.5 mg, and its estimated purity by LCMS analysis was 88.5%. Analysis condition 1: Retention time=1.45 min; ESI-MS(+) m/z [M+3H]²⁺: 789.2.

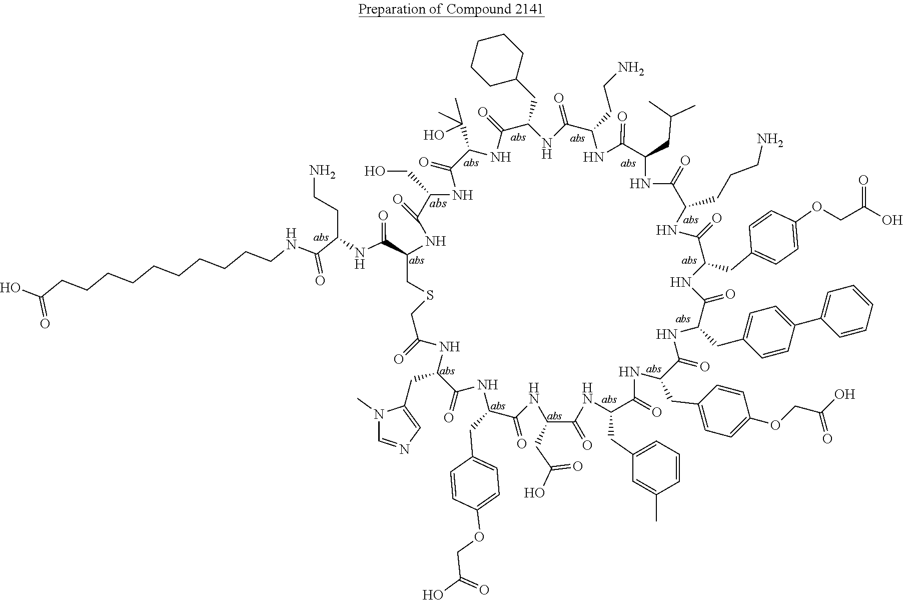 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cncn3C)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2141 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH and Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.9 mg, and its estimated purity by LCMS analysis was 94.6%. Analysis condition: Retention time=1.64 min; ESI-MS(+) m/z [M+2H]²⁺: 1221.2.

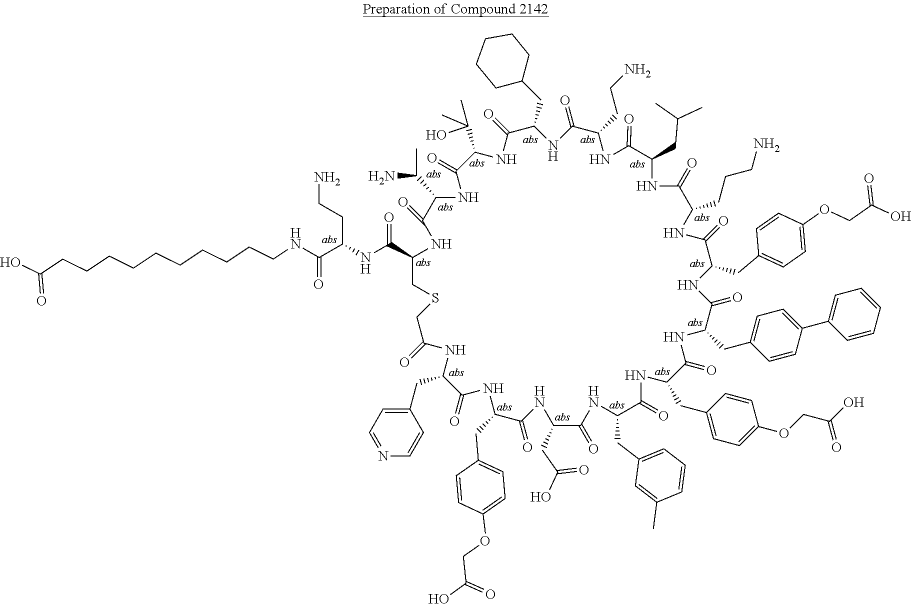 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H]([C@H](C)N)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2142 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.4 mg, and its estimated purity by LCMS analysis was 90.2%. Analysis condition: Retention time=1.65 min; ESI-MS(+) m/z [M+2H]²⁺: 1220.4.

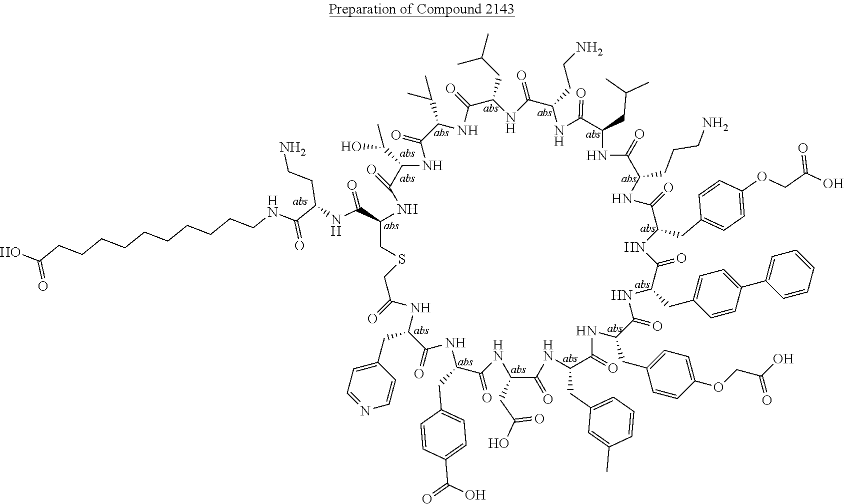 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2143 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 9% B, 9-49% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4.3 mg, and its estimated purity by LCMS analysis was 95.1%. Analysis condition: Retention time=1.58 min; ESI-MS(+) m/z [M+2H]²⁺: 1184.2.

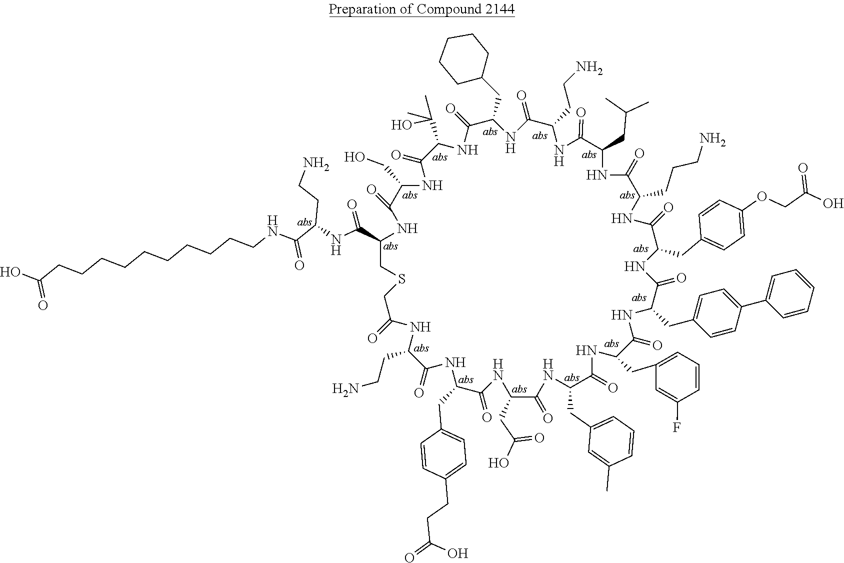 [SMILES: Cc1cccc(C[C@@H]2NC(=O)[C@H](CC(=O)O)NC(=O)[C@H](Cc3ccc(CCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3cccc(F)c3)NC2=O)c1]

Compound 2144 was prepared on a 30 μmol scale. The yield of the product was 26 mg, and its estimated purity by LCMS analysis was 100%. Analysis condition A: Retention time=1.68 min; ESI-MS(+) m/z [M+2H]²⁺: 1168.1.

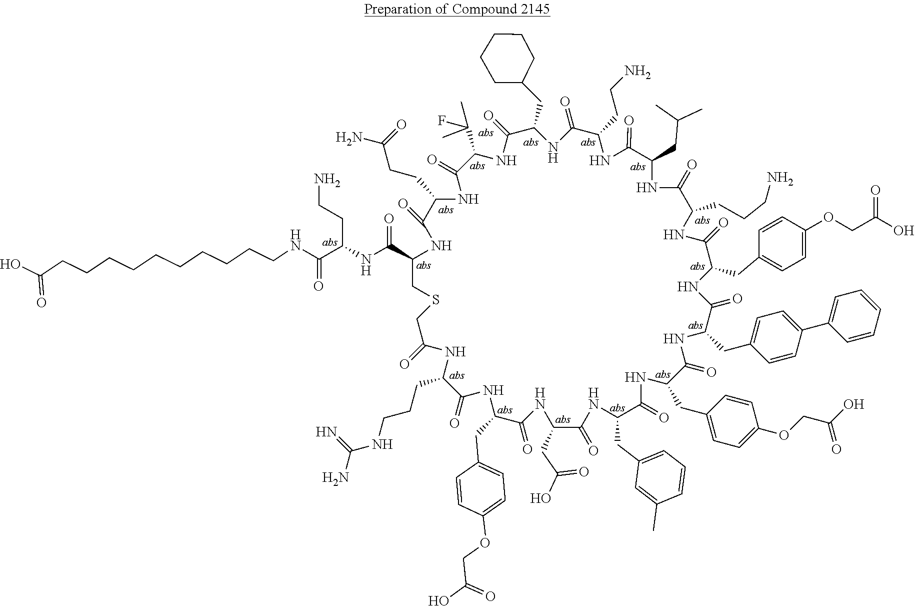 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)(C)F)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2145 was prepared on a 50 μmol scale. The yield of the product was 13.3 mg, and its estimated purity by LCMS analysis was 100%. Analysis condition A: Retention time=1.37 min; ESI-MS(+) m/z [M+2H]²⁺: 831.1.

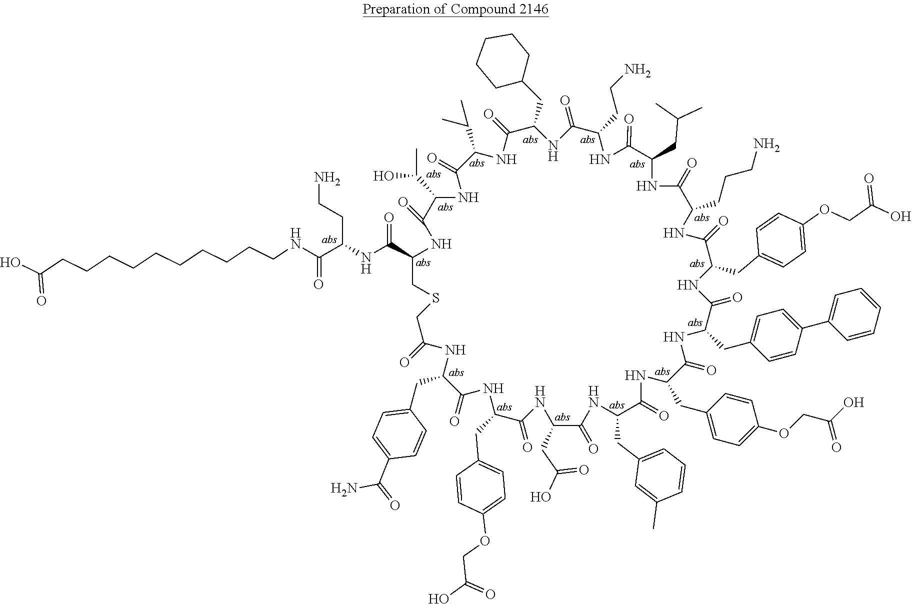 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2146 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-CONH₂)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 11% B, 11-51% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.5 mg, and its estimated purity by LCMS analysis was 91.1%. Analysis condition: Retention time=1.72 min; ESI-MS(+) m/z [M+2H]²⁺: 1240.3.

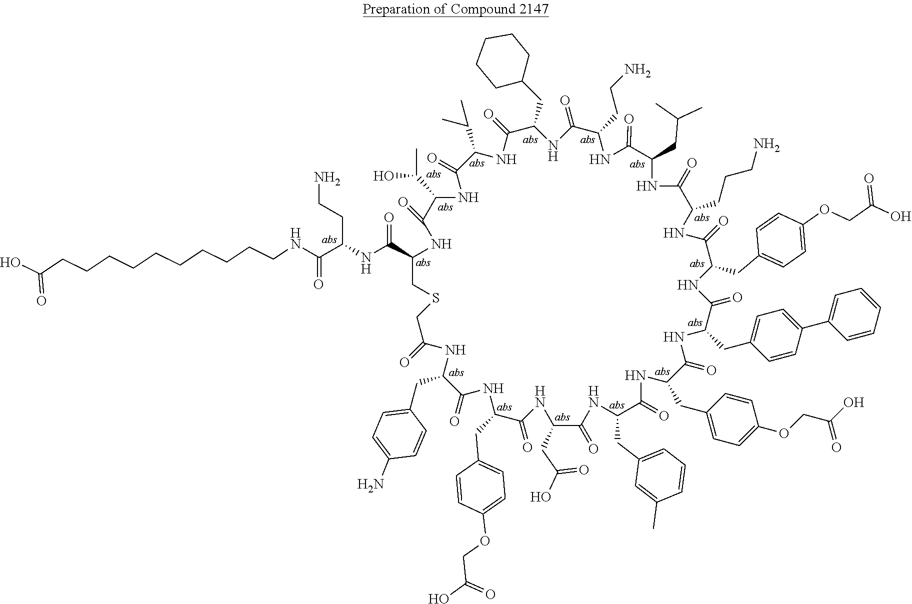 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(N)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2147 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (4-NHBoc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 21 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.7 mg, and its estimated purity by LCMS analysis was 96.2%. Analysis condition: Retention time=1.67 min; ESI-MS(+) m/z [M+2H]²⁺: 1226.

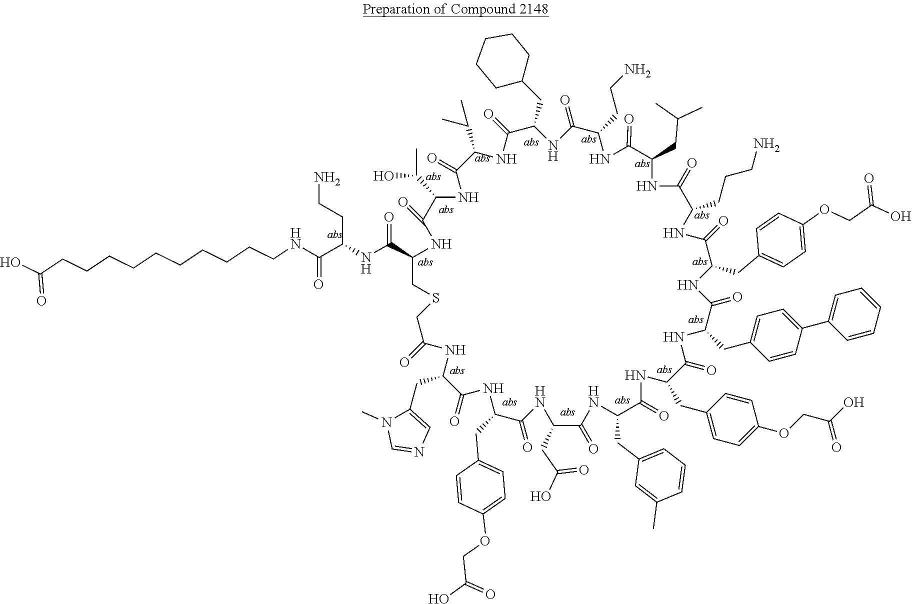 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cncn3C)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2148 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 1.7 mg, and its estimated purity by LCMS analysis was 92.2%. Analysis condition: Retention time=1.68 min; ESI-MS(+) m/z [M+3H]³⁺: 813.9.

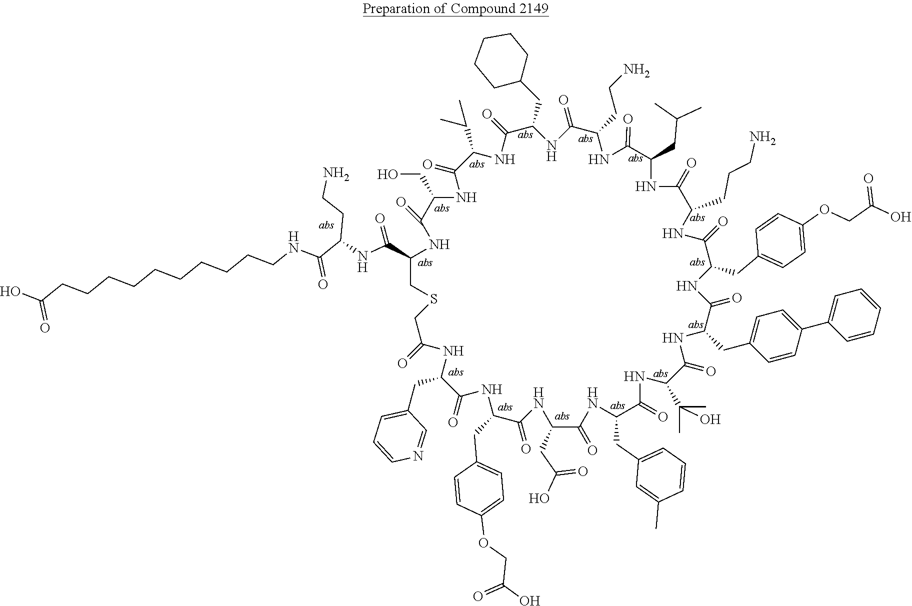 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)O)NC2=O)c1]

Compound 2149 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH and Fmoc-Ala (3-pyridyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 22 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.5 mg, and its estimated purity by LCMS analysis was 100%. Analysis condition: Retention time=1.55 min; ESI-MS(+) m/z [M+2H]²⁺: 1159.1.

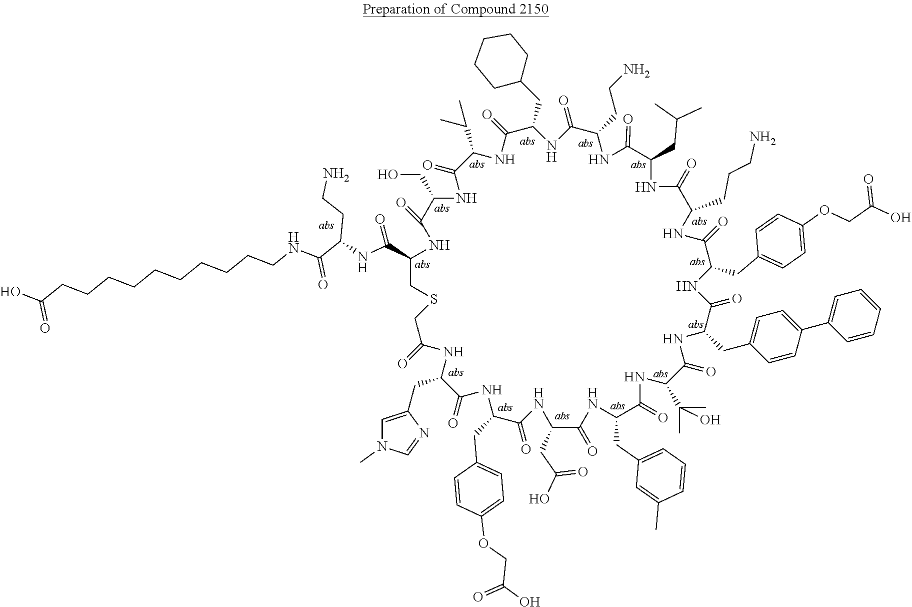 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cn(C)cn3)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)O)NC2=O)c1]

Compound 2150 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH and Na-(((9H-fluoren-9-yl) methoxy) carbonyl)-Nt-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 27% B, 27-67% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.8 mg, and its estimated purity by LCMS analysis was 96.9%. Analysis condition: Retention time=1.77 min; ESI-MS(+) m/z [M+2H]²⁺: 1161.2.

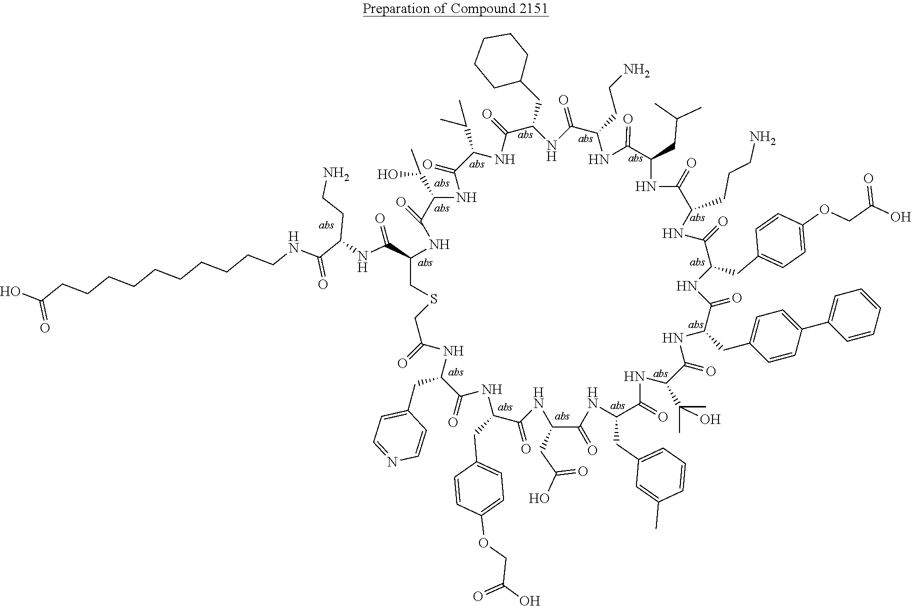 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)O)NC2=O)c1]

Compound 2151 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-57% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 5.2 mg, and its estimated purity by LCMS analysis was 88.7%. Analysis condition 2: Retention time=1.5 min; ESI-MS(+) m/z [M+2H]²⁺: 1166.3.

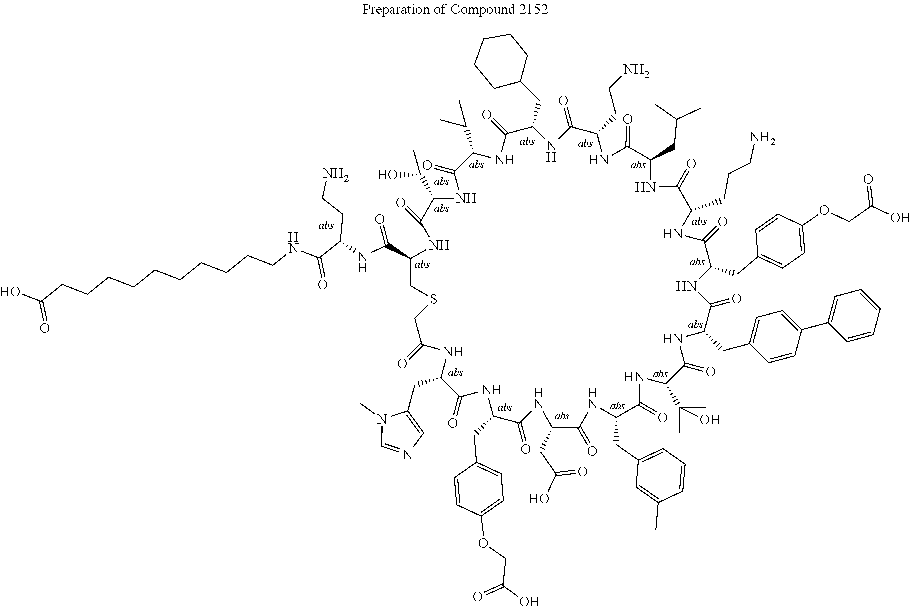 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cncn3C)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)O)NC2=O)c1]

Compound 2152 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH and Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.1 mg, and its estimated purity by LCMS analysis was 93.9%. Analysis condition 1: Retention time=1.44 min; ESI-MS(+) m/z [M+2H]²⁺: 1167.2.

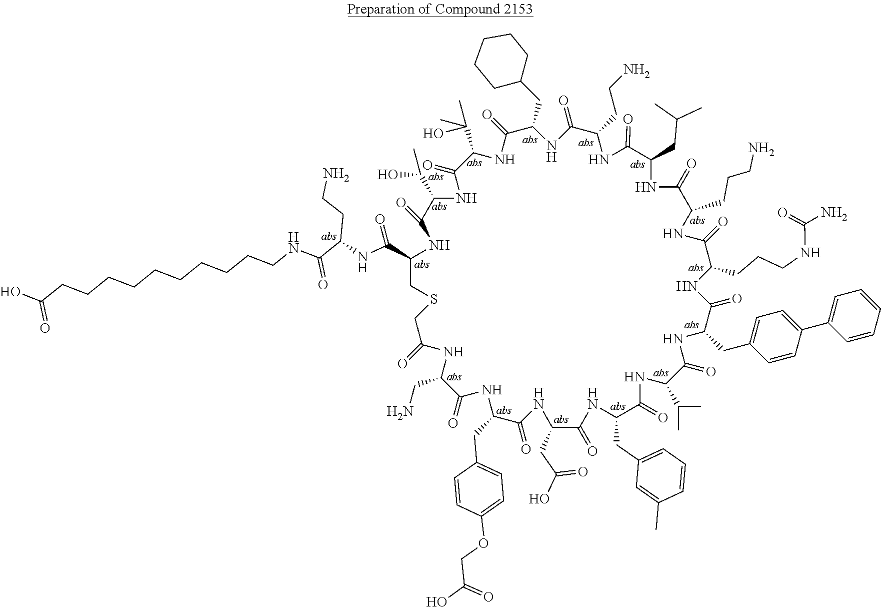

Compound 2153 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH and Na-(((9H-fluoren-9-yl) methoxy) carbonyl)-Np-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 16.1 mg, and its estimated purity by LCMS analysis was 90%. Analysis condition 2: Retention time=1.63 min; ESI-MS(+) m/z [M+2H]²⁺: 1103.

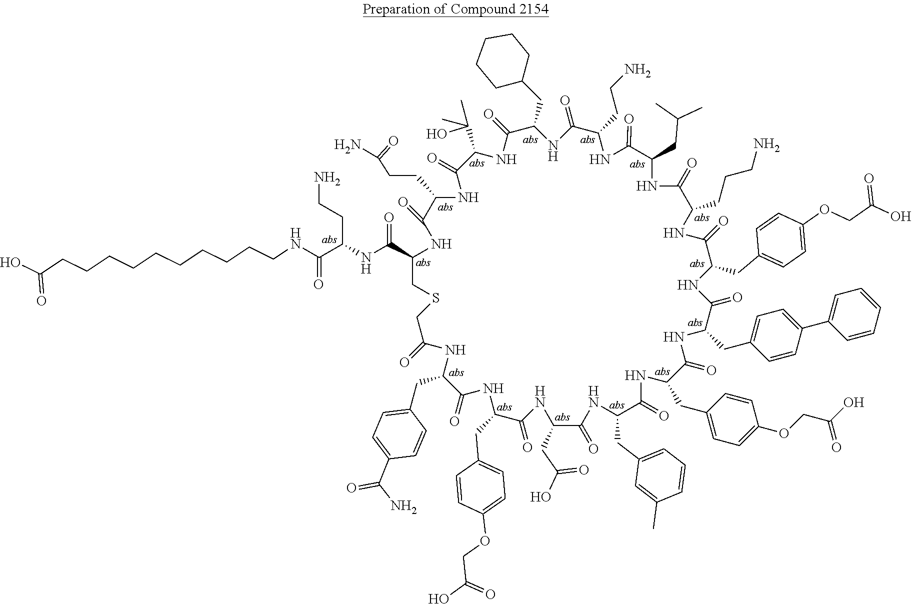 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2154 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH and Fmoc-Phe (4-CONH₂)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 35.7 mg, and its estimated purity by LCMS analysis was 88.8%. Analysis condition 1: Retention time=1.43 min; ESI-MS(+) m/z [M+2H]²⁺: 1261.8.

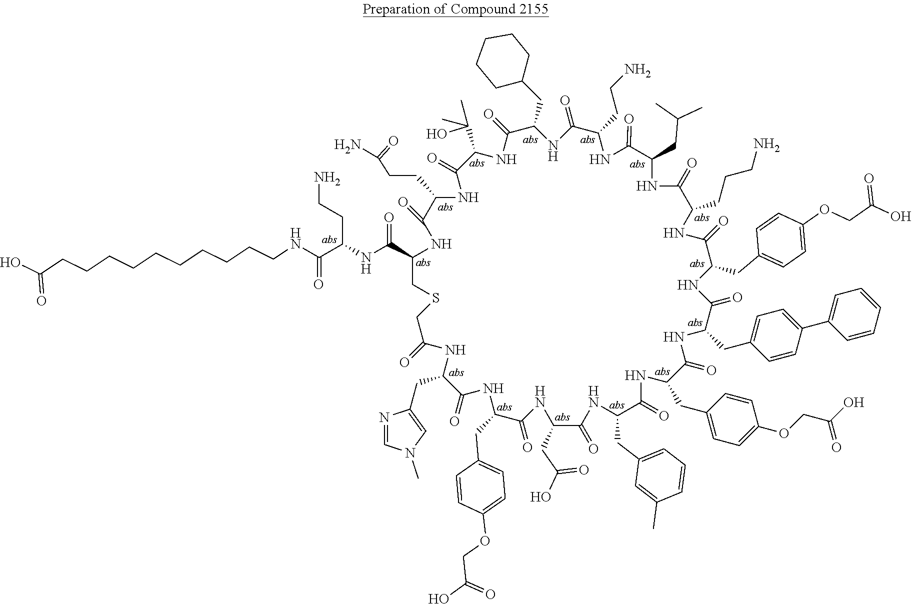 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cn(C)cn3)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2155 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH and Na-(((9H-fluoren-9-yl) methoxy) carbonyl)-Nt-methyl-L-histidine; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 10.3 mg, and its estimated purity by LCMS analysis was 90.8%. Analysis condition 1: Retention time=1.46 min; ESI-MS(+) m/z [M+2H]²⁺: 1242.1.

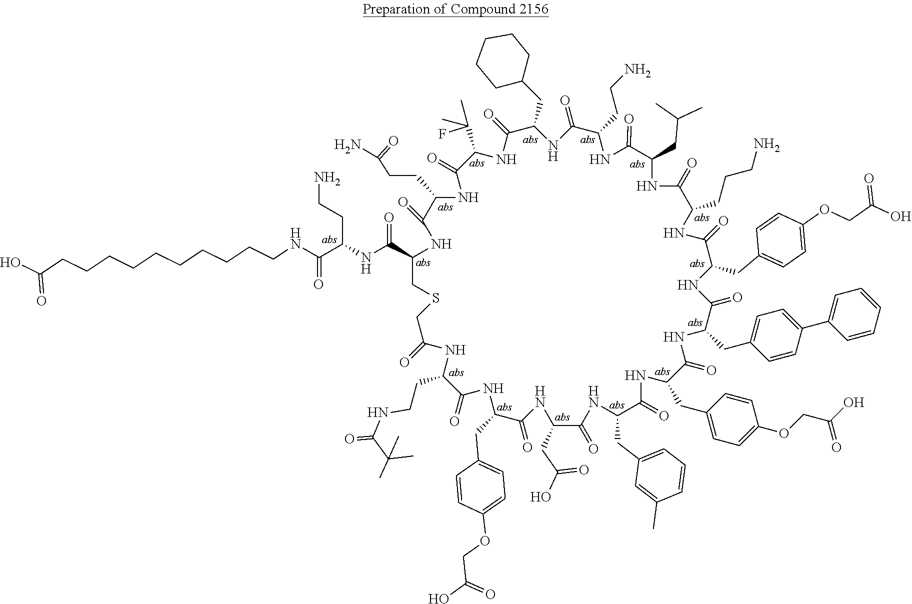 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)C(C)(C)C)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)(C)F)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2156 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH and Fmoc-Dab(COtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.7 mg, and its estimated purity by LCMS analysis was 87.1%. Analysis condition 2: Retention time=1.76 min; ESI-MS(+) m/z [M+3H]³⁺: 840.2.

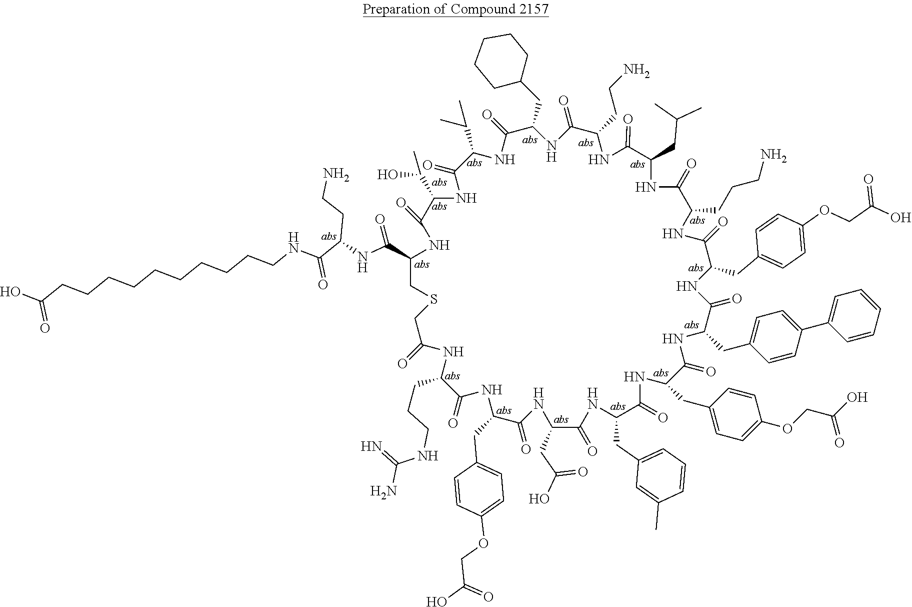 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2157 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: Waters CSH Fluoro Phenyl, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 4-minute hold at 100% B; Flow Rate: 25 mL/min; Column Temperature: 25 C. Fraction collection was triggered by MS and UV signals. Fractions containing the desired product were combined and dried via centrifugal evaporation. The yield of the product was 1.9 mg, and its estimated purity by LCMS analysis was 86%. Analysis condition 2: Retention time=1.46 min; ESI-MS(+) m/z [M+2H]²⁺: 1214.

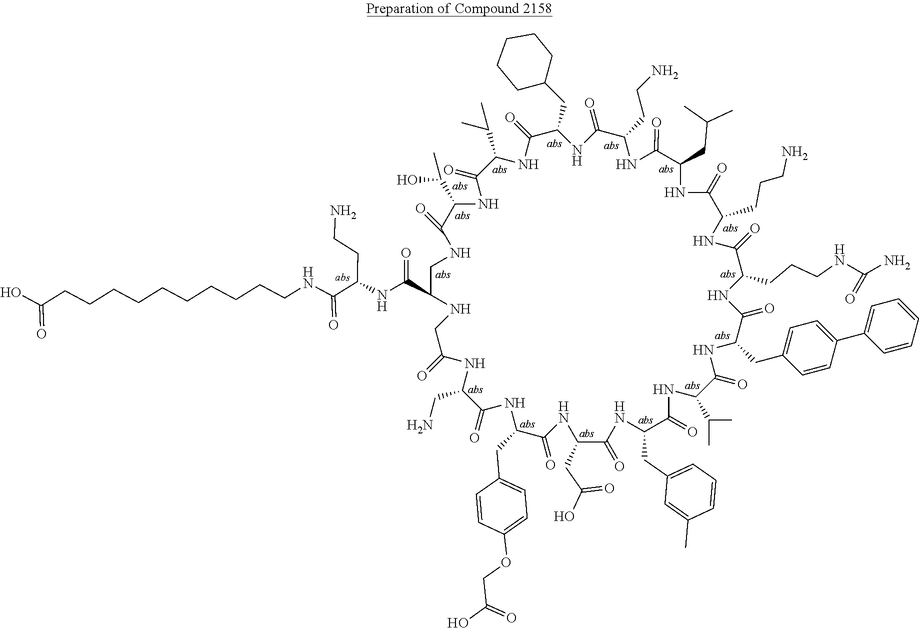 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CN[C@@H](C(=O)N[C@@H](CCN)C(=O)NCCCCCCCCCCC(=O)O)CNC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2158 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: Waters CSH Fluoro Phenyl, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 2-minute hold at 100% B; Flow Rate: 25 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-52% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.6 mg, and its estimated purity by LCMS analysis was 100%. Analysis condition 1: Retention time=1.69 min; ESI-MS(+) m/z [M+2H]²⁺: 724.1.

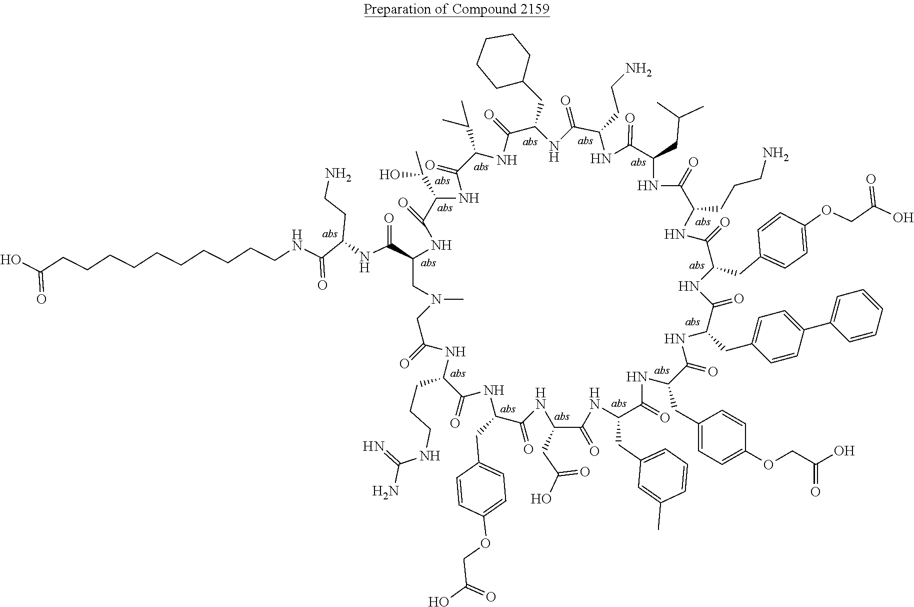 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CN(C)C[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2159 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-F)—OH and Fmoc-NMe-Gly-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: Waters CSH Fluoro Phenyl, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 25 mL/min; Column Temperature: 25 C. Fraction collection was triggered by MS and UV signals. Fractions containing the desired product were combined and dried via centrifugal evaporation. The yield of the product was 1.9 mg, and its estimated purity by LCMS analysis was 83.8% as a mixture of diastereomers. Analysis condition 2: Retention time=1.48, 1.52 min; ESI-MS(+) m/z [M+2H]²⁺: 1222.04, 1221.08.

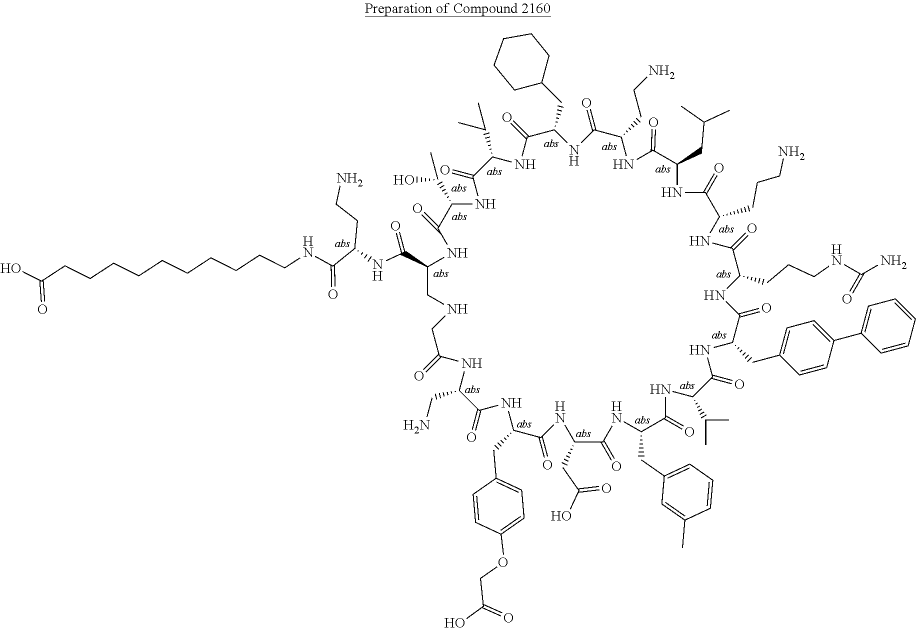 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CNC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2160 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: Waters CSH Fluoro Phenyl, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 2-minute hold at 100% B; Flow Rate: 25 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.1 mg, and its estimated purity by LCMS analysis was 100%. Analysis condition 1: Retention time=1.7 min; ESI-MS(+) m/z [M+3H]³⁺: 725.1.

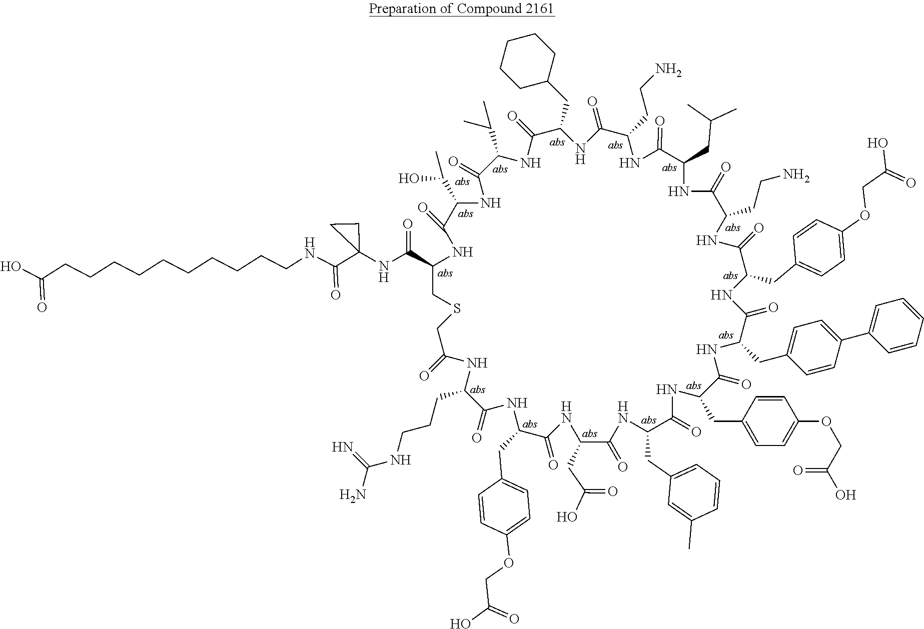 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NC3(C(=O)NCCCCCCCCCCC(=O)O)CC3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2161 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.4 mg, and its estimated purity by LCMS analysis was 97.7%. Analysis condition 2: Retention time=1.75 min; ESI-MS(+) m/z [M+3H]³⁺: 805.2.

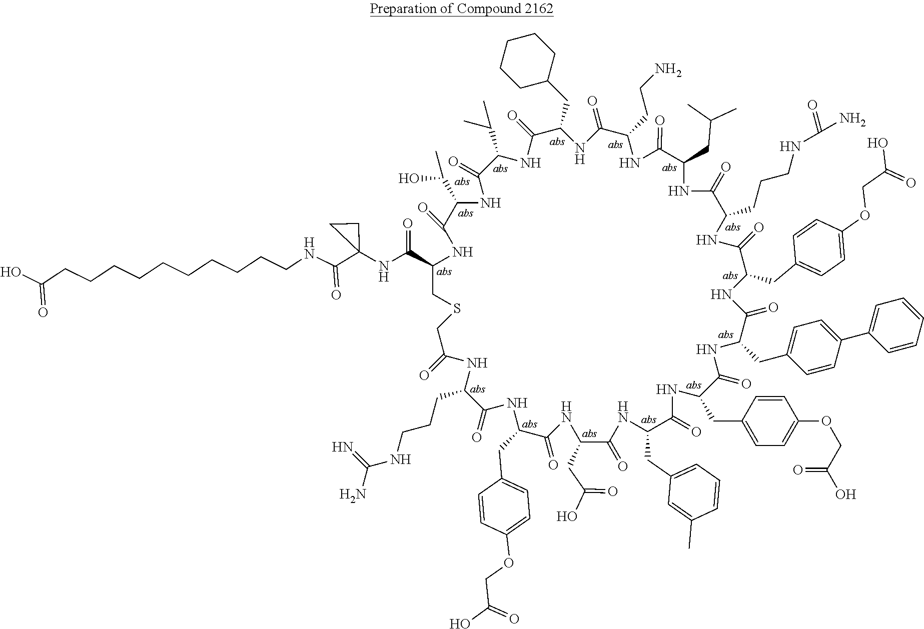 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NC3(C(=O)NCCCCCCCCCCC(=O)O)CC3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2162 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 23% B, 23-63% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 32.4 mg, and its estimated purity by LCMS analysis was 95.2%. Analysis condition 1: Retention time=1.52 min; ESI-MS(+) m/z [M+2H]²⁺: 1236.1.

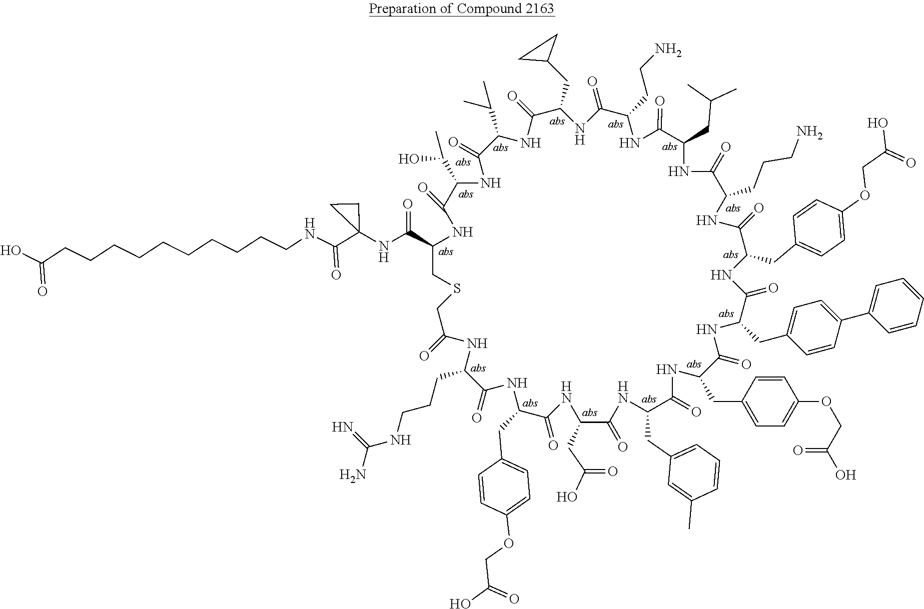 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NC3(C(=O)NCCCCCCCCCCC(=O)O)CC3)NC(=O)[C@H]([C@@H](C)O)NC(=O)[C@H](C(C)C)NC(=O)[C@H](CC3CC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2163 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclopropyl)-OH and 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 10% B, 10-50% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 11.4 mg, and its estimated purity by LCMS analysis was 95.1%. Analysis condition 1: Retention time=1.44 min; ESI-MS(+) m/z [M+3H]³⁺: 796.1.

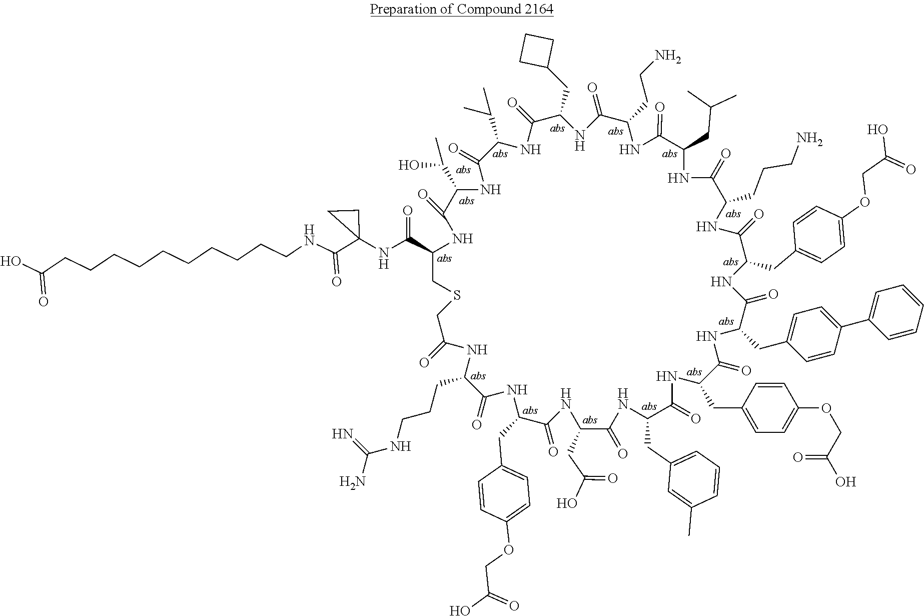 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NC3(C(=O)NCCCCCCCCCCC(=O)O)CC3)NC(=O)[C@H]([C@@H](C)O)NC(=O)[C@H](C(C)C)NC(=O)[C@H](CC3CCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2164 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclobutyl)-OH and 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 11% B, 11-51% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 16.4 mg, and its estimated purity by LCMS analysis was 96.7%. Analysis condition 1: Retention time=1.53 min; ESI-MS(+) m/z [M+2H]²⁺: 1200.

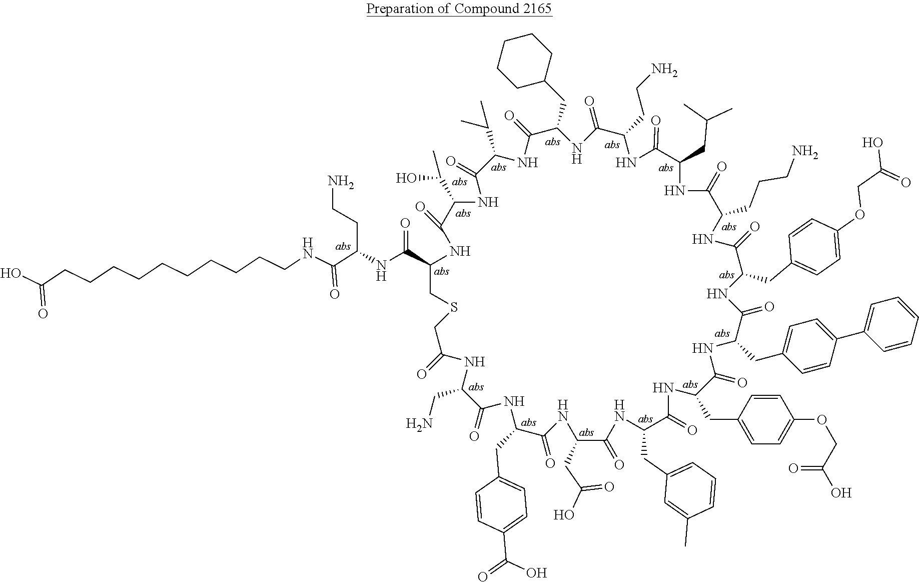 [SMILES: Cc1cccc(C[C@@H]2NC(=O)[C@H](CC(=O)O)NC(=O)[C@H](Cc3ccc(C(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2165 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 24 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.6 mg, and its estimated purity by LCMS analysis was 94.9%. Analysis condition 1: Retention time=1.54 min; ESI-MS(+) m/z [M+3H]³⁺: 782.

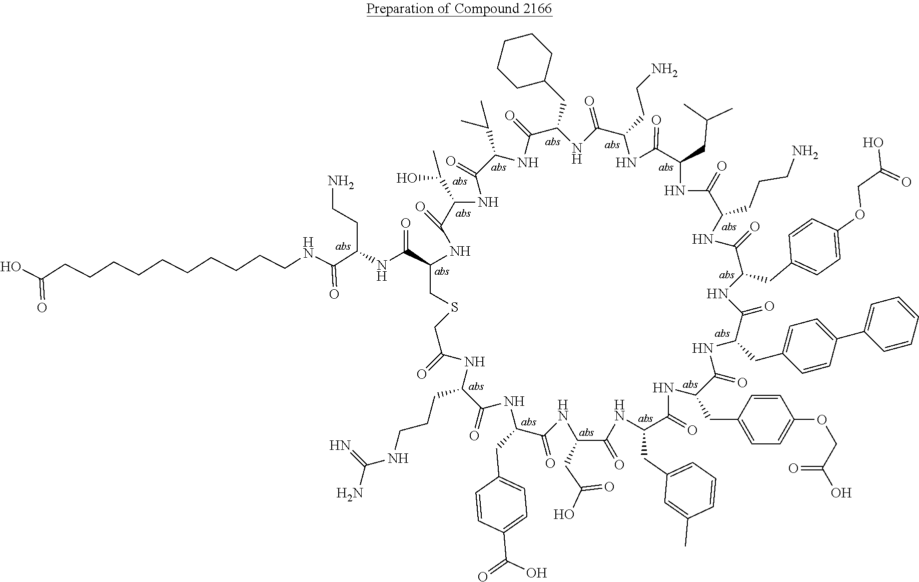 [SMILES: Cc1cccc(C[C@@H]2NC(=O)[C@H](CC(=O)O)NC(=O)[C@H](Cc3ccc(C(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2166 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 8.6 mg, and its estimated purity by LCMS analysis was 95.5%. Analysis condition 1: Retention time=1.55 min; ESI-MS(+) m/z [M+3H]³⁺: 806.1.

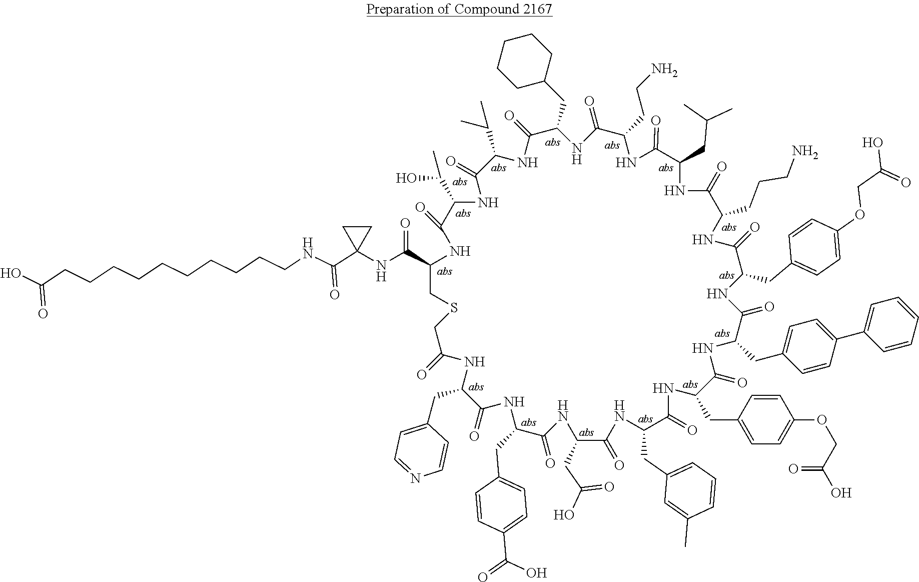 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)NC3(C(=O)NCCCCCCCCCCC(=O)O)CC3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2167 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 65.3 mg, and its estimated purity by LCMS analysis was 91%. Analysis condition 1: Retention time=1.59 min; ESI-MS(+) m/z [M+3H]³⁺: 797.1.

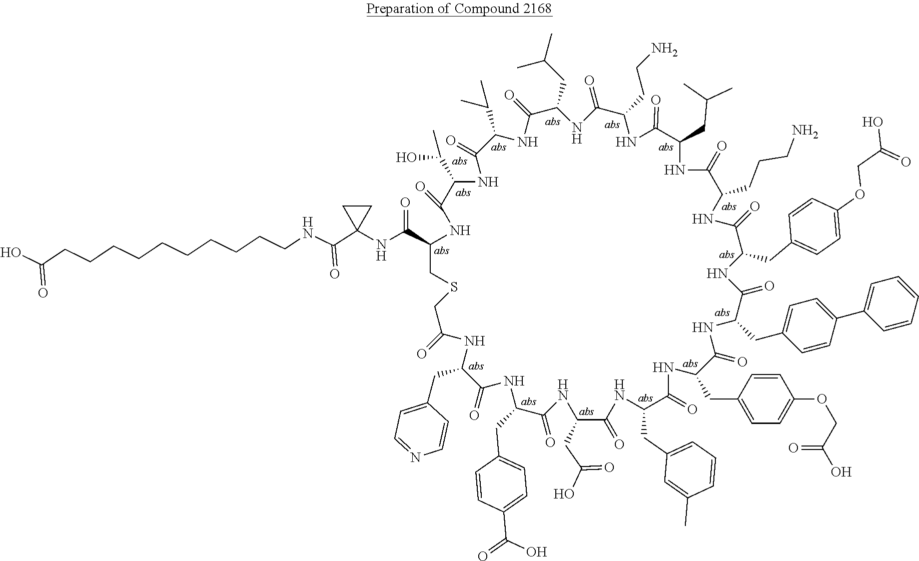 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)NC3(C(=O)NCCCCCCCCCCC(=O)O)CC3)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2168 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with 1-((((9H-fluoren-9-yl) methoxy) carbonyl)amino)cyclopropane-1-carboxylic acid; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×50 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 8% B, 8-48% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 27.2 mg, and its estimated purity by LCMS analysis was 96.2%. Analysis condition 1: Retention time=1.48 min; ESI-MS(+) m/z [M+3H]³⁺: 784.

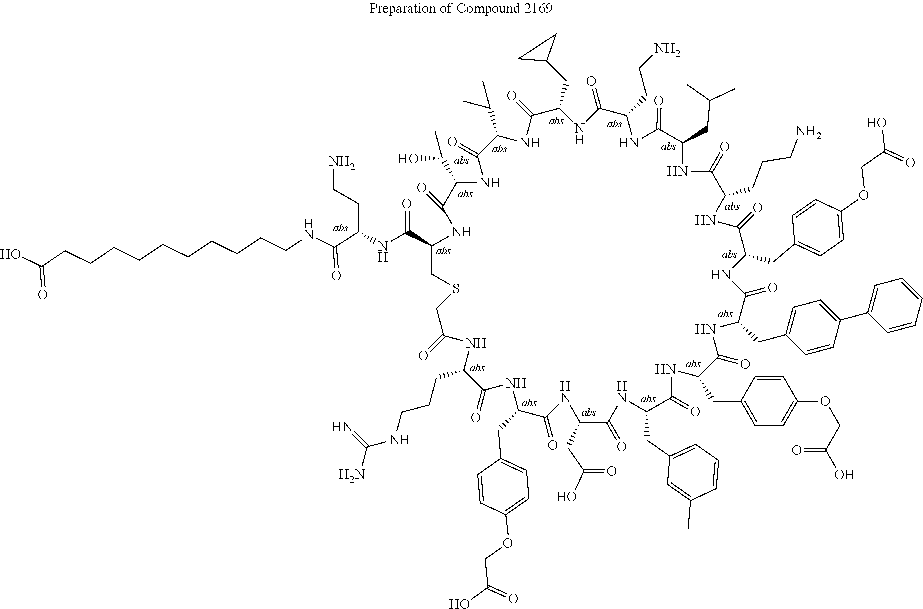 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2169 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclopropyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 5% B, 5-45% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4.9 mg, and its estimated purity by LCMS analysis was 100%. Analysis condition 2: Retention time=1.35 min; ESI-MS(+) m/z [M+3H]³⁺: 801.6.

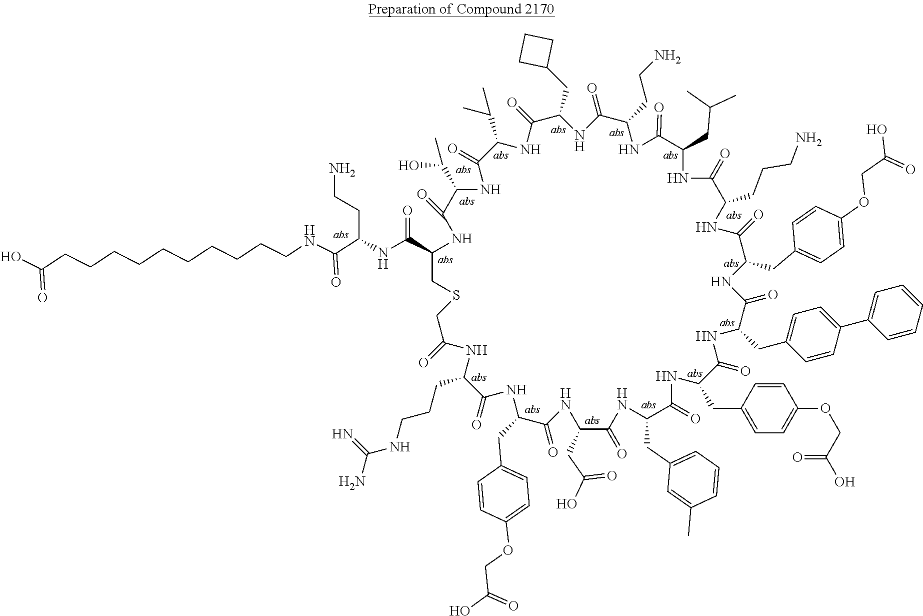 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2170 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclobutyl)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 24 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.5 mg, and its estimated purity by LCMS analysis was 81.8%. Analysis condition 1: Retention time=1.43 min; ESI-MS(+) m/z [M+3H]³⁺: 806.

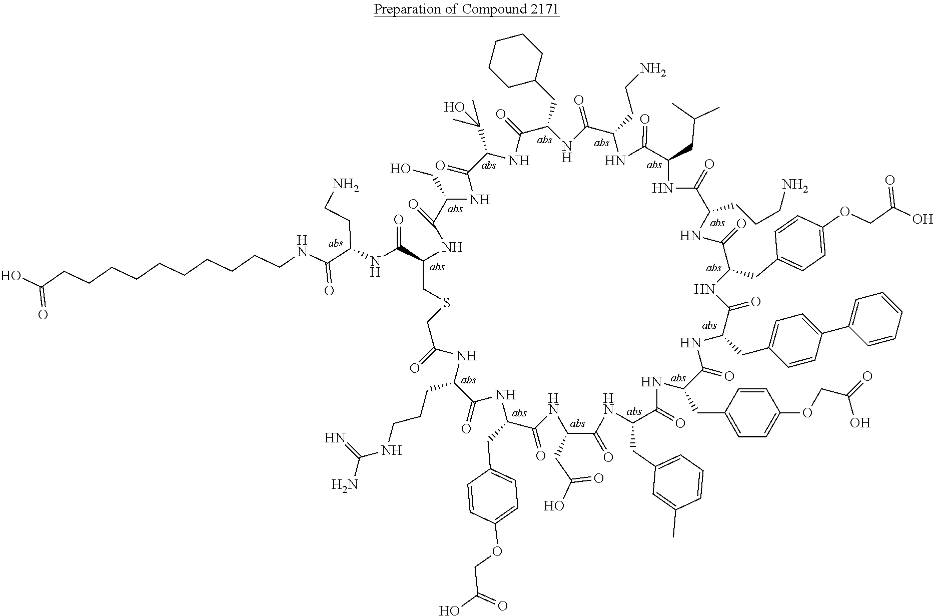 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2171 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-50% B over 26 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 43.3 mg, and its estimated purity by LCMS analysis was %. Analysis condition 1: Retention time=1.51 min; ESI-MS(+) m/z [M+2H]²⁺: 1224.2.

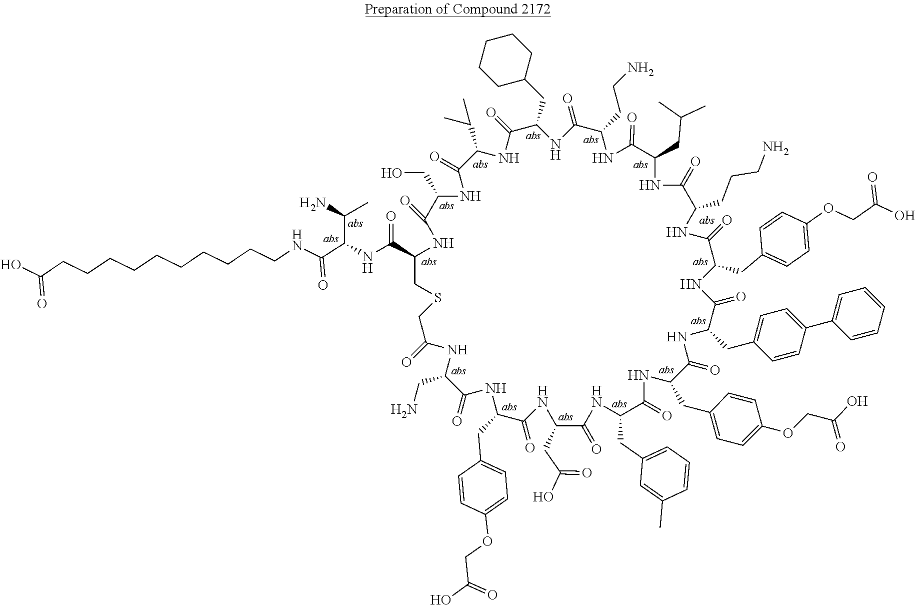 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@H](C(=O)NCCCCCCCCCCC(=O)O)[C@H](C)N)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2172 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of composed of the following general procedures: The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.6 mg, and its estimated purity by LCMS analysis was 95.1%. Analysis condition 2: Retention time=1.55 min; ESI-MS(+) m/z [M+2H]²⁺: 1181.1.

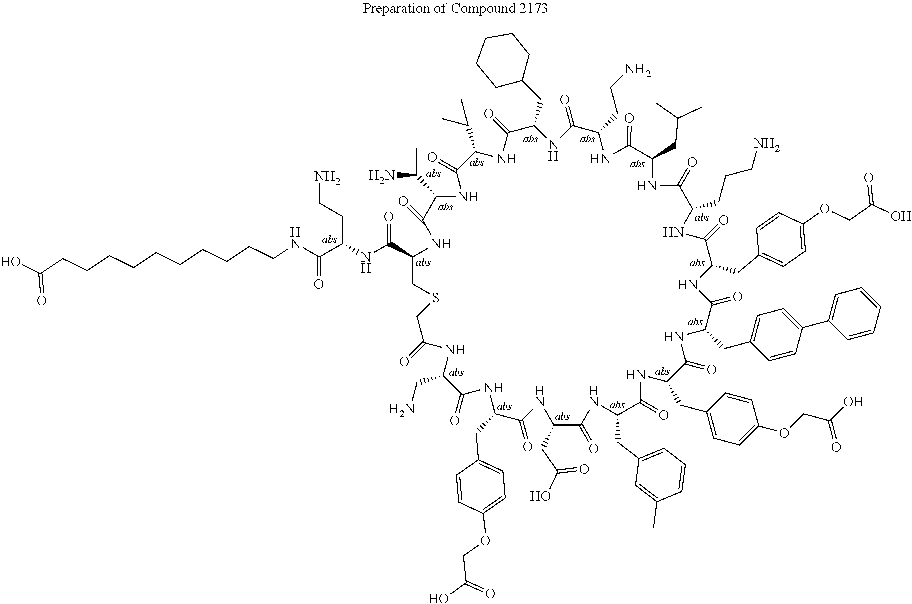 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@H](C)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2173 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-β-(S)-Me-Dap(Boc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 1.7 mg, and its estimated purity by LCMS analysis was 94.8%. Analysis condition 2: Retention time=1.63 min; ESI-MS(+) m/z [M+2H]²⁺: 792.2.

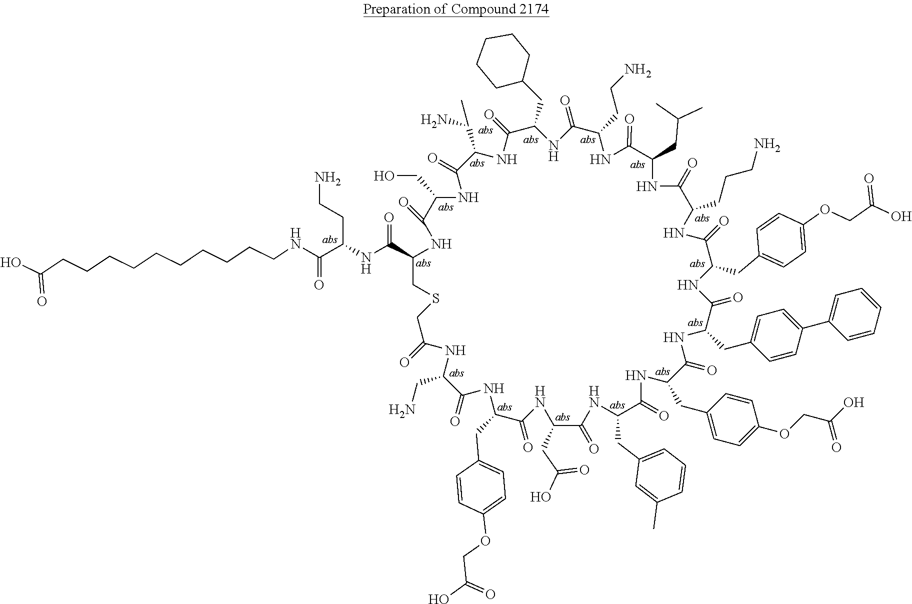 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H]([C@H](C)N)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2174 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-β-(S)-Me-Dap(Boc)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 16% B, 16-56% B over 20 minutes, then a 0-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 10 mg, and its estimated purity by LCMS analysis was 97.6%. Analysis condition 1: Retention time=1.58 min; ESI-MS(+) m/z [M+2H]²⁺: 1182.

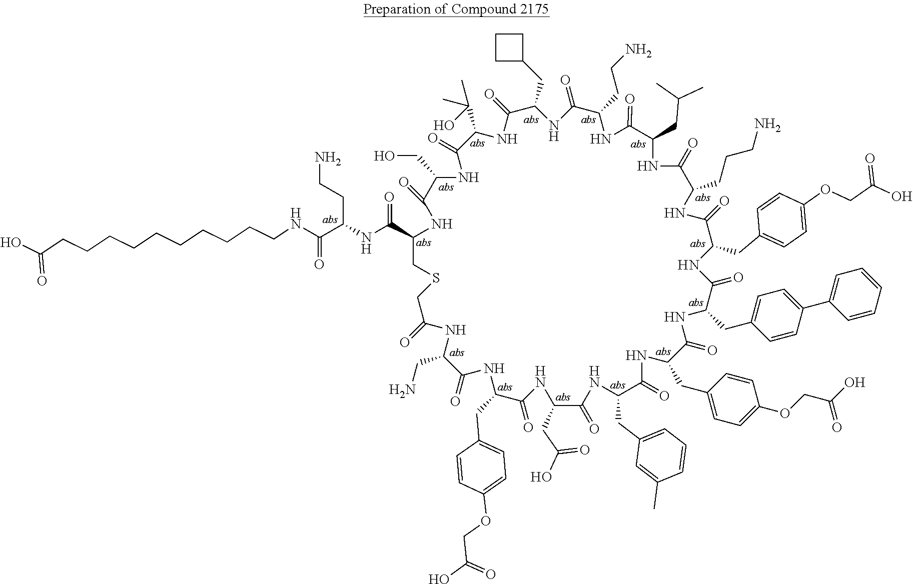 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2175 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Val(β-OH)—OH and Fmoc-D-Ala(cyclobutyi)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 11% B, 11-51% B over 20 minutes, then a 0-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 6.4 mg, and its estimated purity by LCMS analysis was 95.2%. Analysis condition 1: Retention time=1.49 min; ESI-MS(+) m/z [M+2H]²⁺: 1174.9.

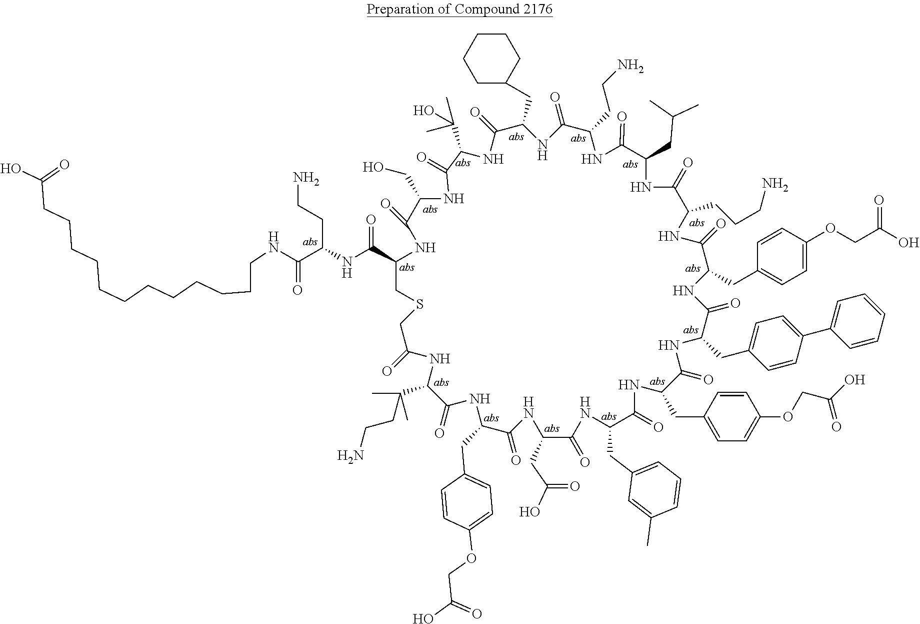 [SMILES: Cc1cccc(C[C@@H]2NC(=O)[C@H](CC(=O)O)NC(=O)[C@H](Cc3ccc(OCC(=O)O)cc3)NC(=O)[C@H](C(C)(C)CCN)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2176 was prepared on a 25 μmol scale. The yield of the product was 3 mg, and its estimated purity by LCMS analysis was 98.9%. Analysis condition A: Retention time=1.61 min; ESI-MS(+) m/z [M+2H]²⁺: 1231.2.

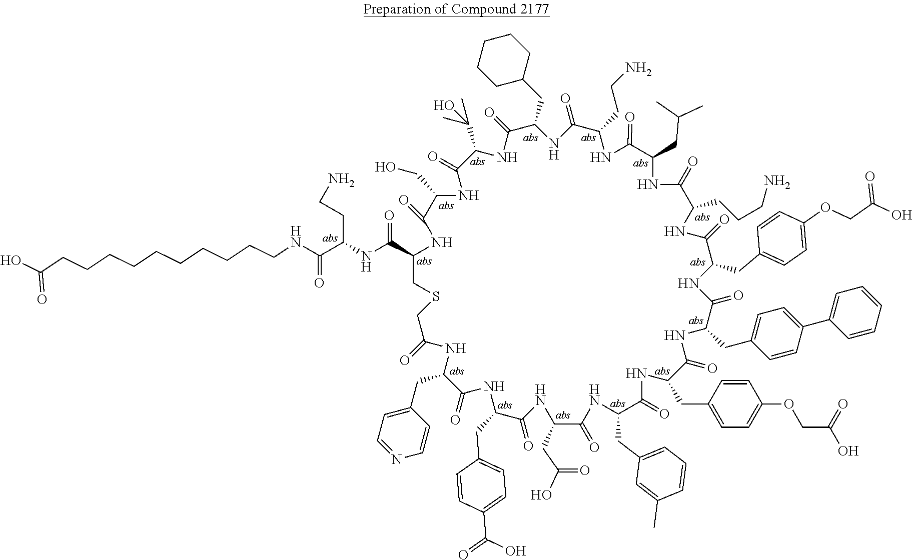 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2177 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Compound 1003 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.4 mg, and its estimated purity by LCMS analysis was 96.1%. Analysis condition 1: Retention time=1.34 min; ESI-MS(+) m/z [M+2H]²⁺: 1205.1.

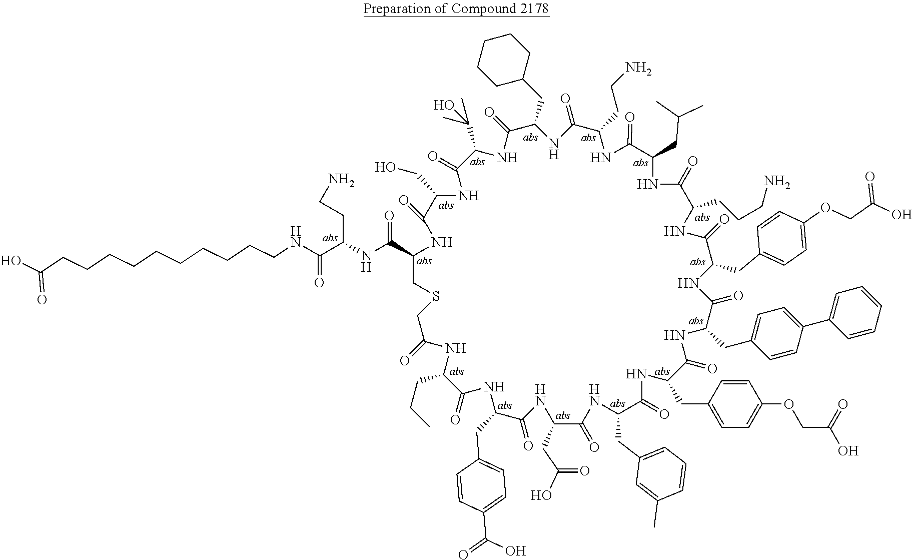 [SMILES: CCC[C@@H]1NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Compound 2178 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Compound 1003 composed of the following general procedures: “Prelude Resin-swelling procedure”, “Prelude Single-coupling procedure”, or ““Prelude Double-coupling procedure”, “Symphony X Resin-swelling procedure”, “Symphony X Single-coupling procedure”, “Symphony X Single-Coupling Manual Addition Procedure A” was followed with Fmoc-Nva-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 14.7 mg, and its estimated purity by LCMS analysis was 96.8%. Analysis condition 1: Retention time=1.56 min; ESI-MS(+) m/z [M+3H]³⁺: 787.

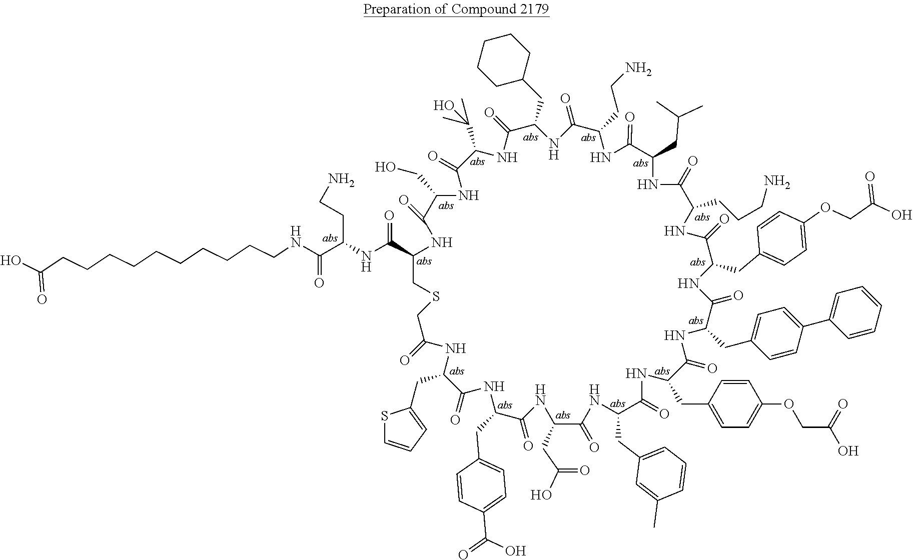 [SMILES: Cc1cccc(C[C@@H]2NC(=O)[C@H](CC(=O)O)NC(=O)[C@H](Cc3ccc(C(=O)O)cc3)NC(=O)[C@H](Cc3cccs3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2179 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Ala (2-thiophene)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 14 mg, and its estimated purity by LCMS analysis was 92.9%. Analysis condition 2: Retention time=1.76 min; ESI-MS(+) m/z [M+2H]²⁺: 1207.1.

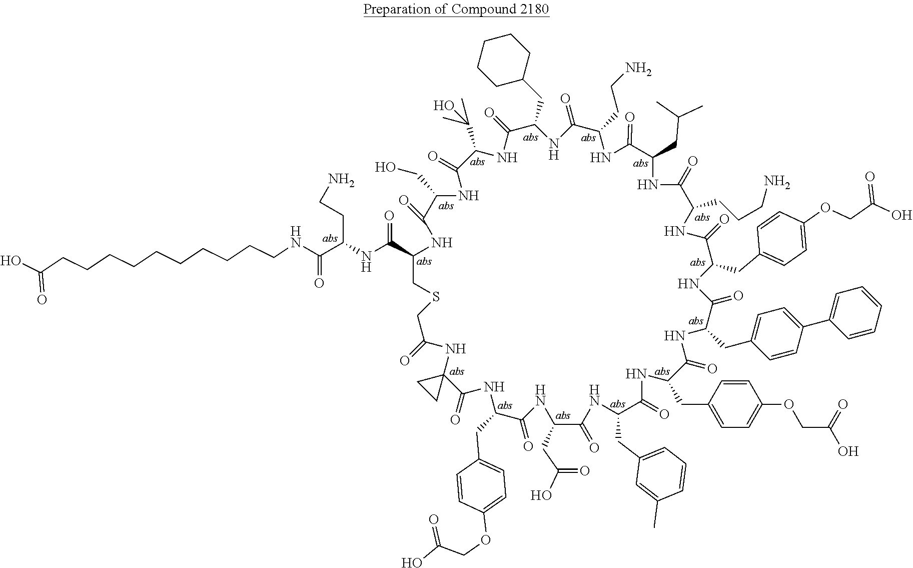 [SMILES: Cc1cccc(C[C@@H]2NC(=O)[C@H](CC(=O)O)NC(=O)[C@H](Cc3ccc(OCC(=O)O)cc3)NC(=O)C3(CC3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2180 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with 1-((((9H-fluoren-9-yl) methoxy) carbonyl)amino)cyclopropane-1-carboxylic acid, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 37 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.6 mg, and its estimated purity by LCMS analysis was 95.7%. Analysis condition 2: Retention time=1.82 min; ESI-MS(+) m/z [M+2H]²⁺: 1187.9.

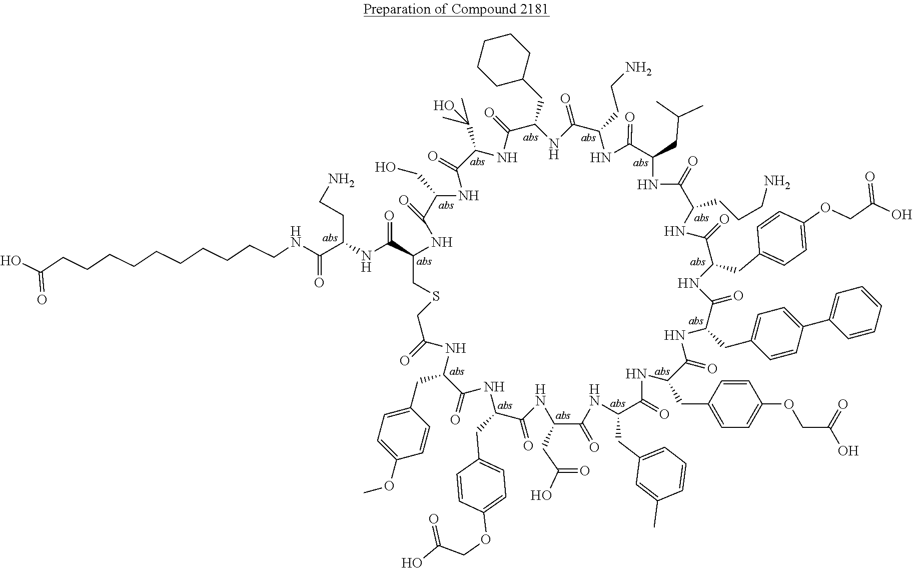 [SMILES: COc1ccc(C[C@@H]2NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC2=O)cc1]

Compound 2181 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (4-OMe)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 24 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.3 mg, and its estimated purity by LCMS analysis was 84.5%. Analysis condition 2: Retention time=1.79 min; ESI-MS(+) m/z [M+2H]²⁺: 1235.9.

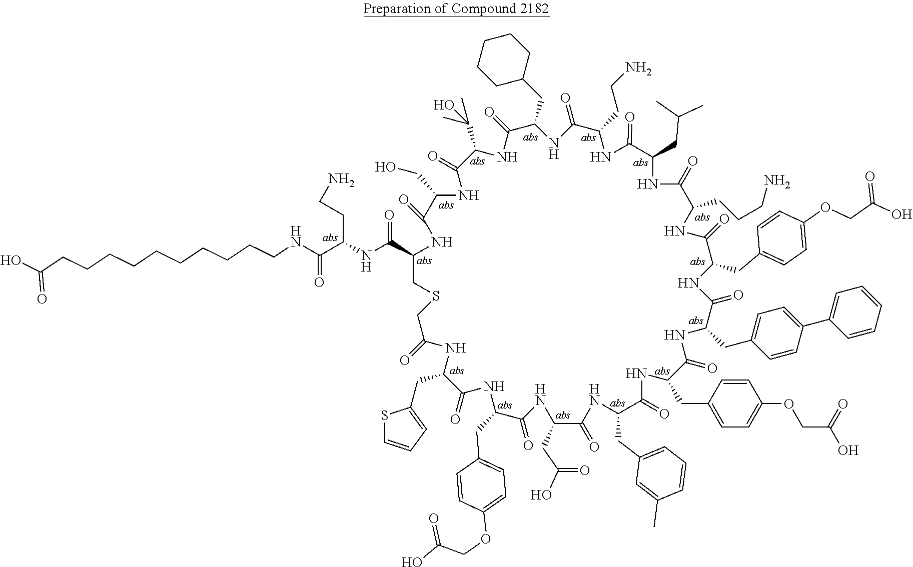 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccs3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2182 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Ala (B-2-thienyl)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.7 mg, and its estimated purity by LCMS analysis was 100%. Analysis condition 1: Retention time=1.57 min; ESI-MS(+) m/z [M+2H]²⁺: 815.

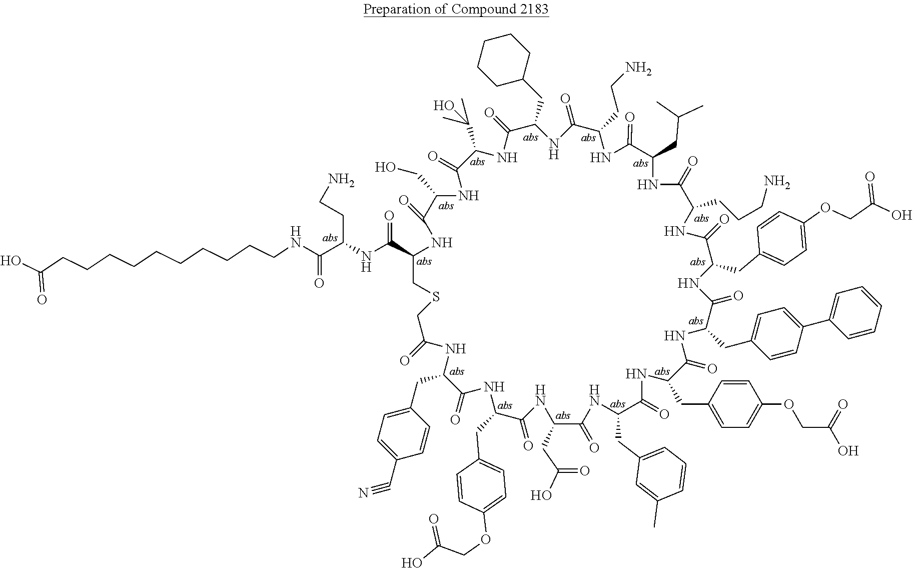 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C#N)cc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2183 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe(4-CN)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 5.6 mg, and its estimated purity by LCMS analysis was 98.3%. Analysis condition 2: Retention time=1.76 min; ESI-MS(+) m/z [M+2H]²⁺: 1232.2.

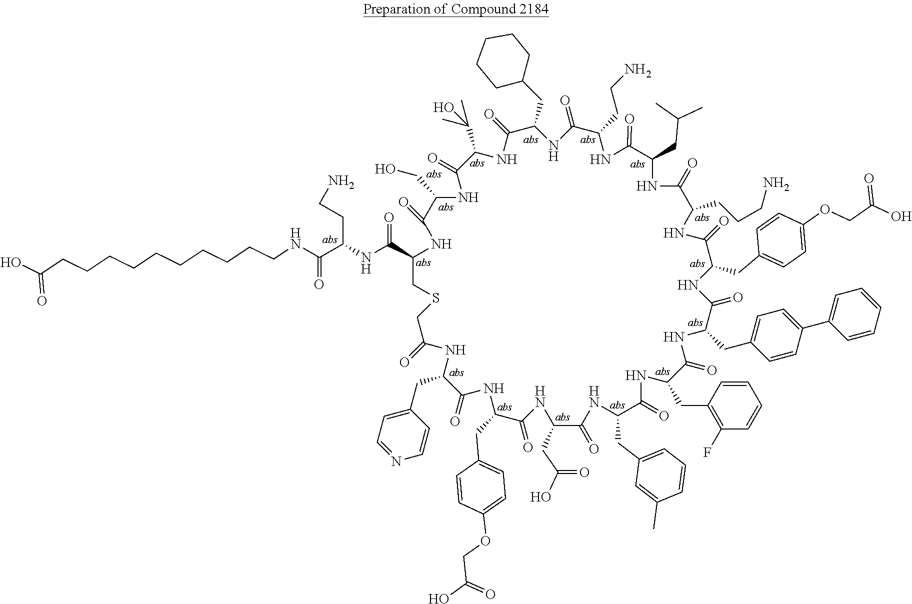 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccccc3F)NC2=O)c1]

Compound 2184 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (2-F)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 36.6 mg, and its estimated purity by LCMS analysis was 95.6%. Analysis condition 2: Retention time=1.53 min; ESI-MS(+) m/z [M+2H]²⁺: 1192.2.

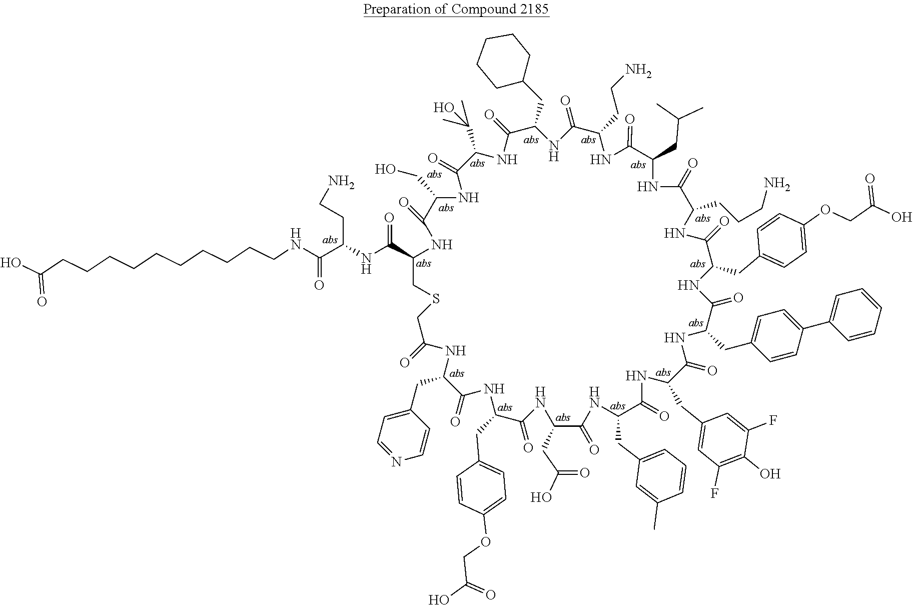 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3cc(F)c(O)c(F)c3)NC2=O)c1]

Compound 2185 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Tyr(2,6-diF)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 6.9 mg, and its estimated purity by LCMS analysis was 90.4%. Analysis condition 2: Retention time=1.6 min; ESI-MS(+) m/z [M+2H]²⁺: 1209.2.

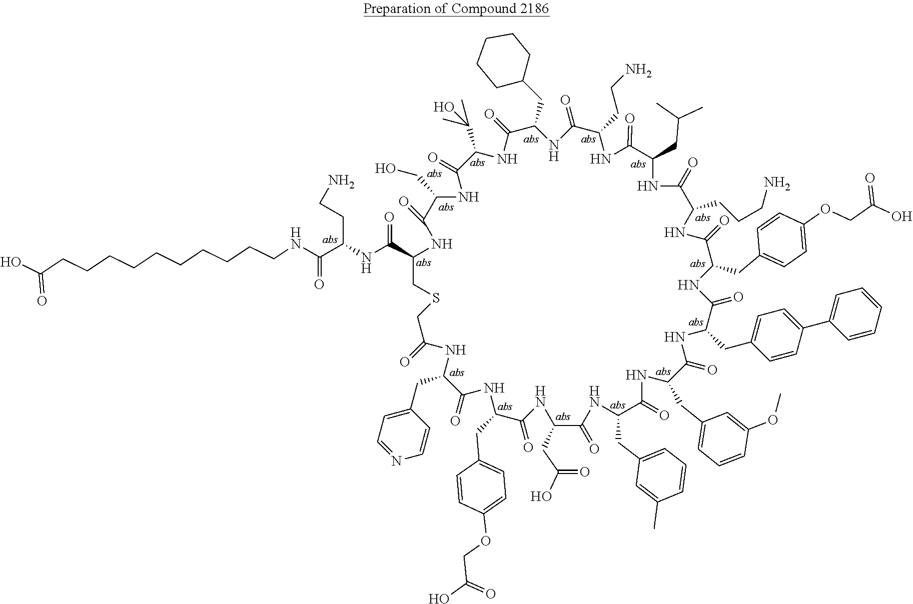 [SMILES: COc1cccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)c1]

Compound 2186 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (3-OMe)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 17% B, 17-56% B over 28 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.8 mg, and its estimated purity by LCMS analysis was 99%. Analysis condition 2: Retention time=1.69 min; ESI-MS(+) m/z [M+3H]³⁺: 799.2.

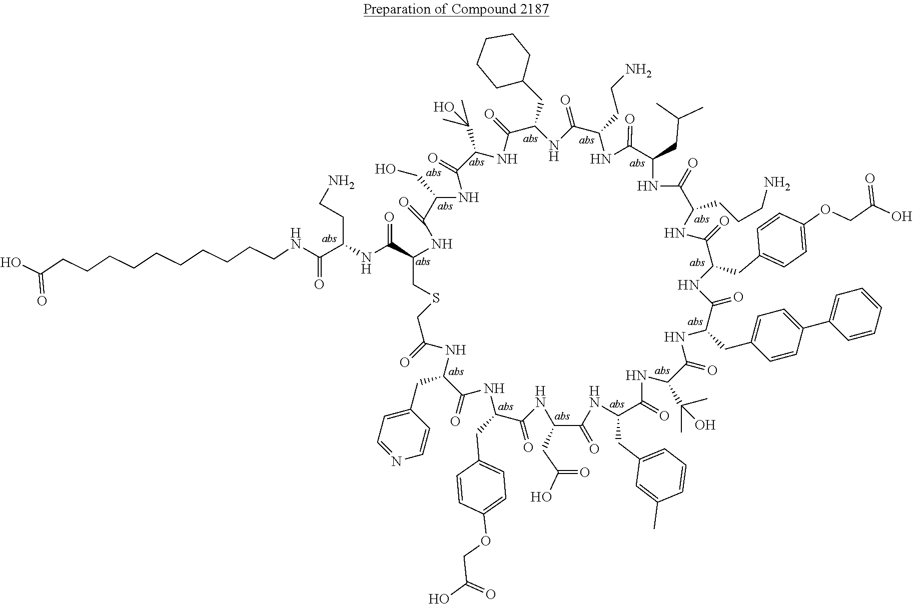 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)(C)O)NC2=O)c1]

Compound 2187 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 6 mg, and its estimated purity by LCMS analysis was 92.3%. Analysis condition 2: Retention time=1.6 min; ESI-MS(+) m/z [M+2H]²⁺: 1167.2.

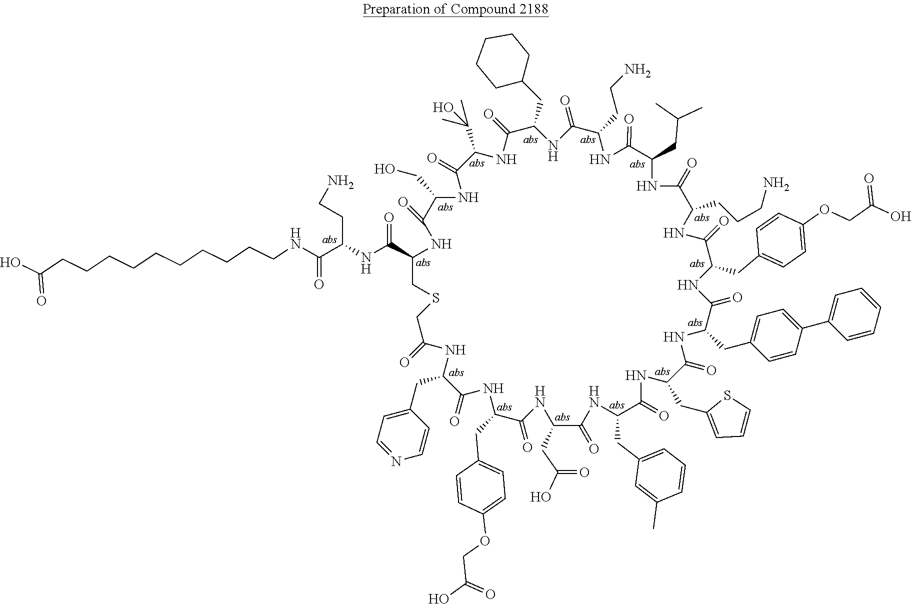 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3cccs3)NC2=O)c1]

Compound 2188 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Ala (2-thienyl)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 24 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.6 mg, and its estimated purity by LCMS analysis was 98.7%. Analysis condition 2: Retention time=1.67 min; ESI-MS(+) m/z [M+2H]²⁺: 1186.2.

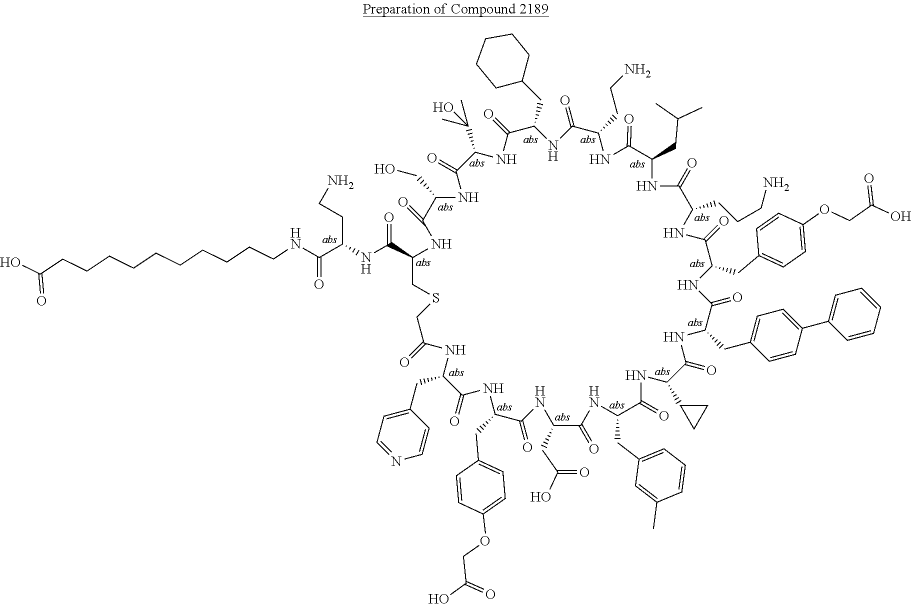 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C3CC3)NC2=O)c1]

Compound 2189 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Gly (cyclopropyl)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.2 mg, and its estimated purity by LCMS analysis was 96%. Analysis condition 1: Retention time=1.62 min; ESI-MS(+) m/z [M+2H]²⁺: 1158.1.

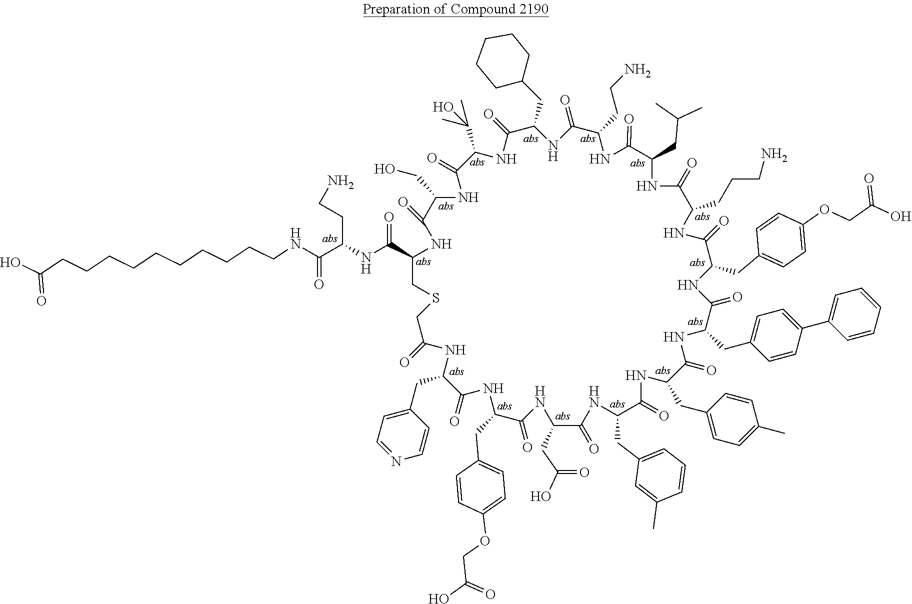 [SMILES: Cc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 2190 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (4-Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 22.1 mg, and its estimated purity by LCMS analysis was 85.6%. Analysis condition 2: Retention time=1.8 min; ESI-MS(+) m/z [M+3H]³⁺: 794.1.

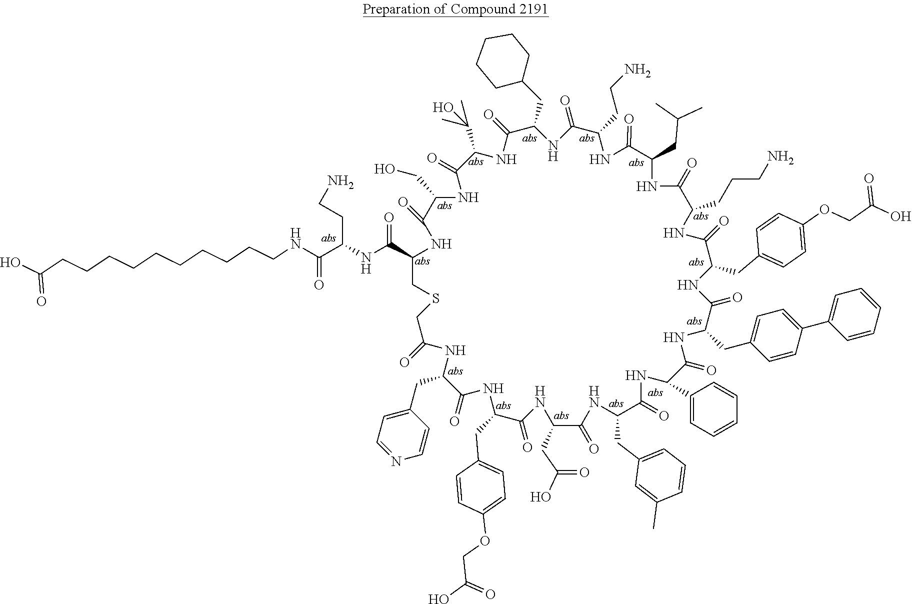 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](c3ccccc3)NC2=O)c1]

Compound 2191 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-phenylglycine-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 3.3 mg, and its estimated purity by LCMS analysis was 99%. Analysis condition 1: Retention time=1.65 min; ESI-MS(+) m/z [M+2H]²⁺: 1176.2.

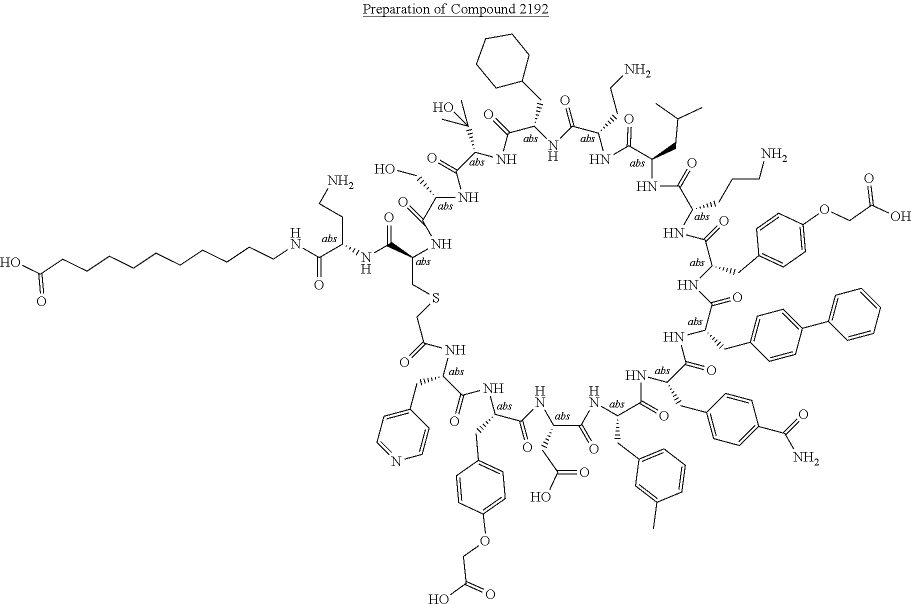 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(N)=O)cc3)NC2=O)c1]

Compound 2192 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe(4-CONH₂)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 1.2 mg, and its estimated purity by LCMS analysis was 100%. Analysis condition 1: Retention time=1.6 min; ESI-MS(+) m/z [M+2H]²⁺: 1204.2.

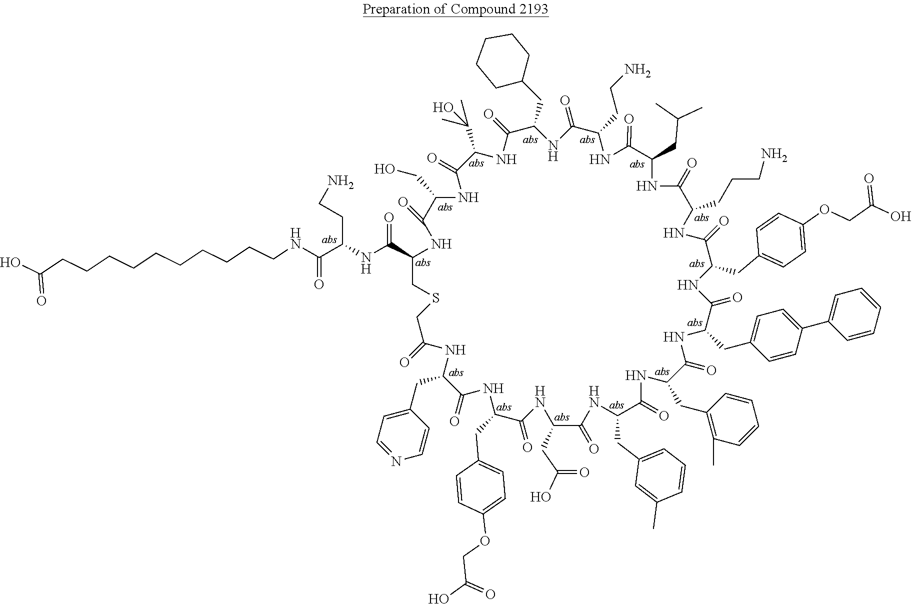 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccccc3C)NC2=O)c1]

Compound 2193 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (2-Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 25 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 3.2 mg, and its estimated purity by LCMS analysis was 95.5%. Analysis condition 1: Retention time=1.74 min; ESI-MS(+) m/z [M+3H]³⁺: 793.9.

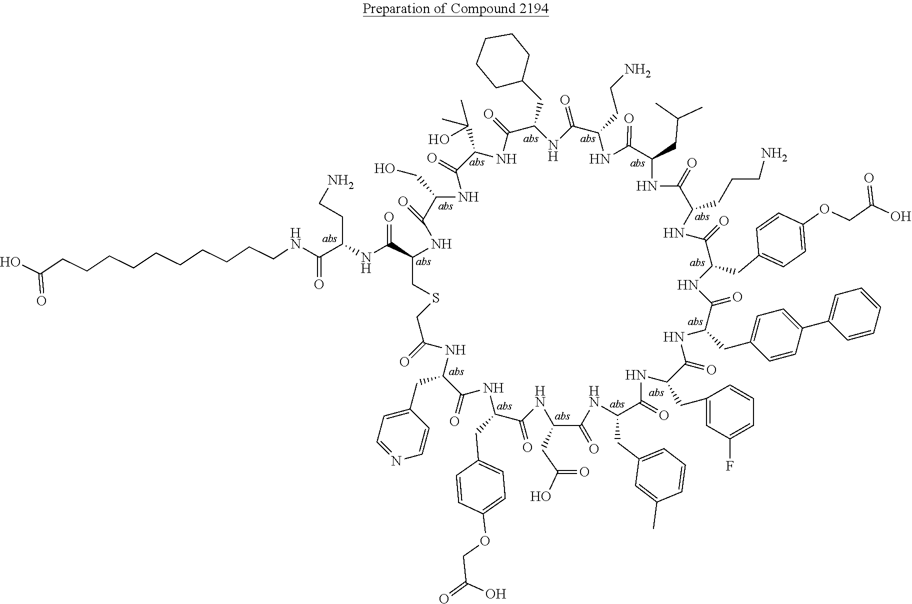 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3cccc(F)c3)NC2=O)c1]

Compound 2194 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe(3-F)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.2 mg, and its estimated purity by LCMS analysis was 95.4%. Analysis condition 2: Retention time=1.7 min; ESI-MS(+) m/z [M+2H]²⁺: 1192.2.

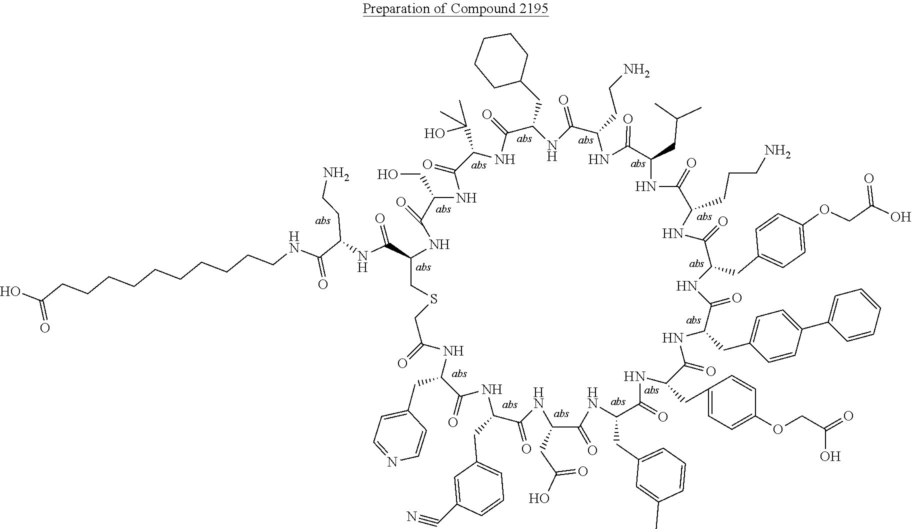 [SMILES: Cc1cccc(C[C@@H]2NC(=O)[C@H](CC(=O)O)NC(=O)[C@H](Cc3cccc(C#N)c3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2195 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (3-CN)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 3.7 mg, and its estimated purity by LCMS analysis was 85.5%. Analysis condition 1: Retention time=1.66 min; ESI-MS(+) m/z [M+3H]³⁺: 797.1.

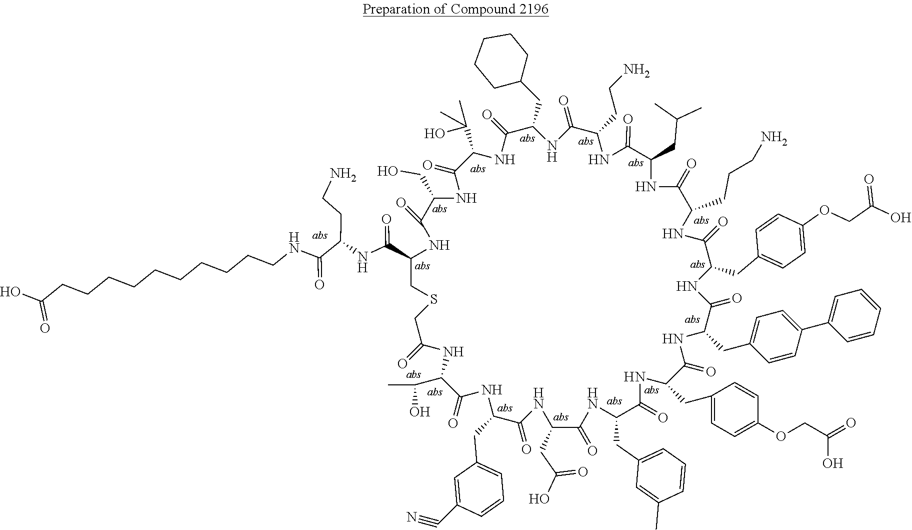 [SMILES: Cc1cccc(C[C@@H]2NC(=O)[C@H](CC(=O)O)NC(=O)[C@H](Cc3cccc(C#N)c3)NC(=O)[C@H]([C@@H](C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2196 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (3-CN)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.5 mg, and its estimated purity by LCMS analysis was 95.9%. Analysis condition 2: Retention time=1.74 min; ESI-MS(+) m/z [M+2H]²⁺: 1172.

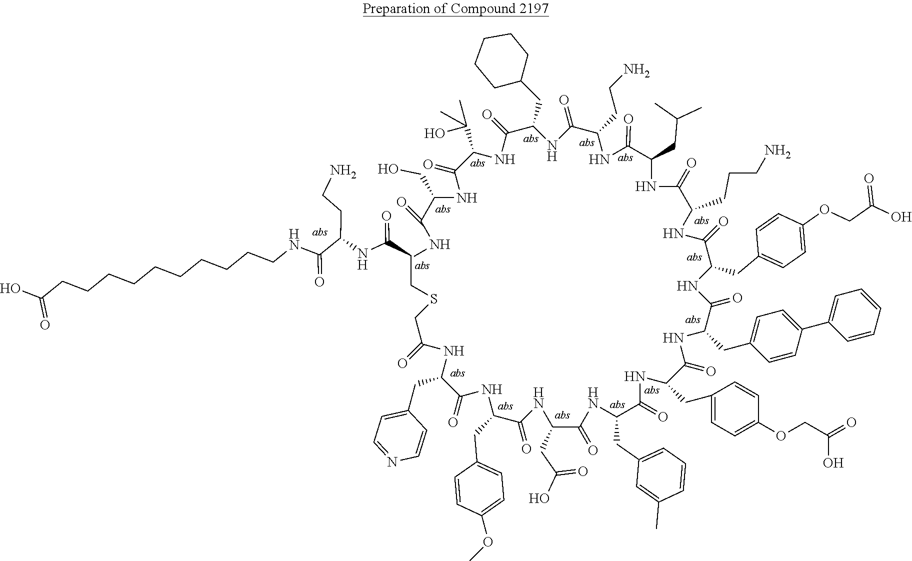

Compound 2197 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (4-OMe)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.3 mg, and its estimated purity by LCMS analysis was 88.9%. Analysis condition 1: Retention time=1.65 min; ESI-MS(+) m/z [M+2H]²⁺: 1198.

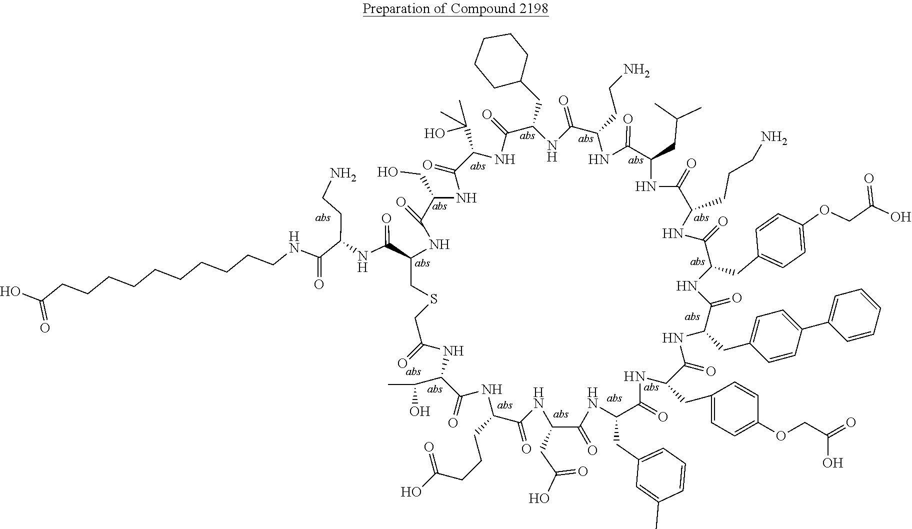 [SMILES: Cc1cccc(C[C@@H]2NC(=O)[C@H](CC(=O)O)NC(=O)[C@H](CCCC(=O)O)NC(=O)[C@H]([C@@H](C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2198 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-(S)-2-((((9H-fluoren-9-yl) methoxy) carbonyl)amino)-6-(tert-butoxy)-6-oxohexanoic acid, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.8 mg, and its estimated purity by LCMS analysis was 86%. Analysis condition 2: Retention time=1.43 min; ESI-MS(+) m/z [M+2H]²⁺: 1157.4.

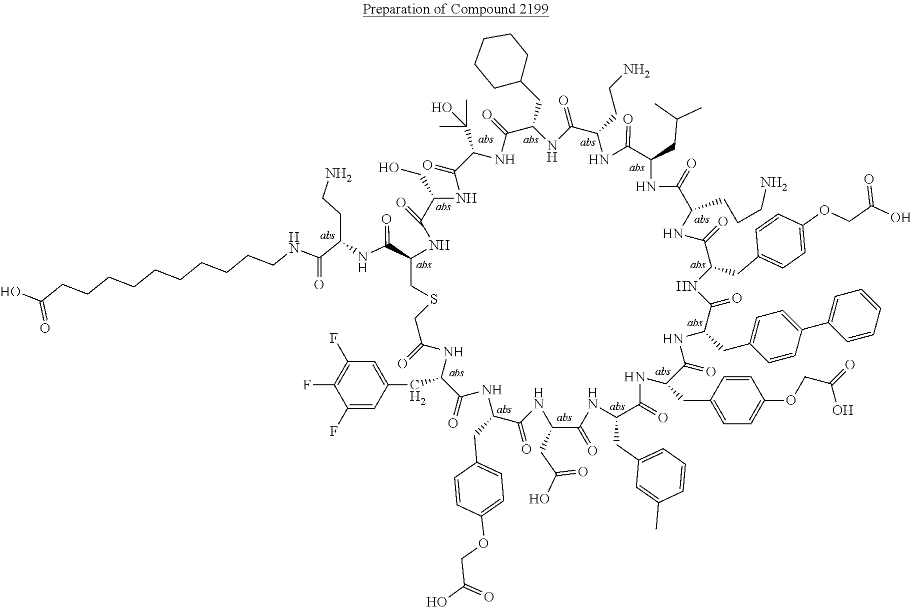 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cc(F)c(F)c(F)c3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

To a 45-mL polypropylene solid-phase reaction vessel was added 2-Chlorotrityl resin pre-loaded with 11-((((9H-fluoren-9-yl) methoxy) carbonyl)amino) undecanoic acid (90 mg, 0.050 mmol), and the reaction vessel was placed on the Prelude peptide synthesizer. The following procedures were then performed sequentially:

“Prelude Resin-swelling procedure” was followed; “Prelude Single-coupling procedure” was followed with Fmoc-Dab(Boc)-OH; “Prelude Single-coupling procedure” was followed with Fmoc-Cys(Trt)-OH; “Prelude Single-coupling procedure” was followed with Fmoc-Ser(tBu)-OH; “Prelude Single-coupling procedure” was followed with Fmoc-Val(β-OH)—OH; “Prelude Single-coupling procedure” was followed with Fmoc-Cha-OH; “Prelude Single-coupling procedure” was followed with Fmoc-Dab(Boc)-OH; “Prelude Single-coupling procedure” or “Prelude double-coupling procedure” was followed with Fmoc-D-Leu-OH; “Prelude Single-coupling procedure” was followed with Fmoc-Orn(Boc)-OH; “Prelude Single-coupling procedure” was followed with Fmoc-Tyr(CH₂COOtBu)-OH; “Prelude Single-coupling procedure” was followed with Fmoc-Bip-OH; “Prelude Single-coupling procedure” was followed with Fmoc-Tyr(CH₂COOtBu)-OH; “Prelude Single-coupling procedure” was followed with Fmoc-Phe (3-Me)-OH; “Prelude Single-coupling procedure” was followed with Fmoc-Asp(tBu)-OH; “Prelude Single-coupling procedure” was followed with Fmoc-Tyr(CH₂COOtBu)-OH; “Prelude Single-Coupling Manual Addition Procedure A” was followed with Phe (3,4,5-triF)—OH; “Prelude Chloroacetic Anhydride coupling procedure” was followed; “Prelude Final rinse and dry procedure” was followed; “Global Deprotection Method A” was followed; “Cyclization Method A” was followed.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 65.6 mg, and its estimated purity by LCMS analysis was 98.5%. Analysis condition 2: Retention time=1.7 min; ESI-MS(+) m/z [M+2H]²⁺: 1246.9.

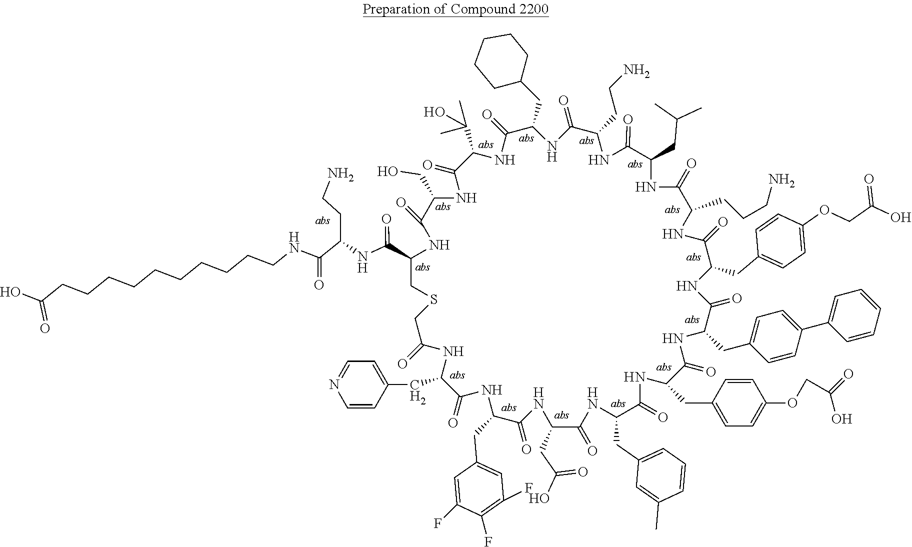 [SMILES: Cc1cccc(C[C@@H]2NC(=O)[C@H](CC(=O)O)NC(=O)[C@H](Cc3cc(F)c(F)c(F)c3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2200 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 2199 composed of the following general procedures: “Prelude Resin-swelling procedure”, “Prelude Single-coupling procedure”, or “Prelude Double-coupling procedure”, “Symphony X Single-coupling procedure”, “Prelude Single-Coupling Manual Addition Procedure A” was followed with Fmoc-Phe (3,4,5-triF)—OH, “Prelude Chloroacetic Anhydride coupling procedure”, “Prelude Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 13.5 mg, and its estimated purity by LCMS analysis was 95.5%. Analysis condition 1: Retention time=1.81 min; ESI-MS(+) m/z [M+2H]²⁺: 1210.2.

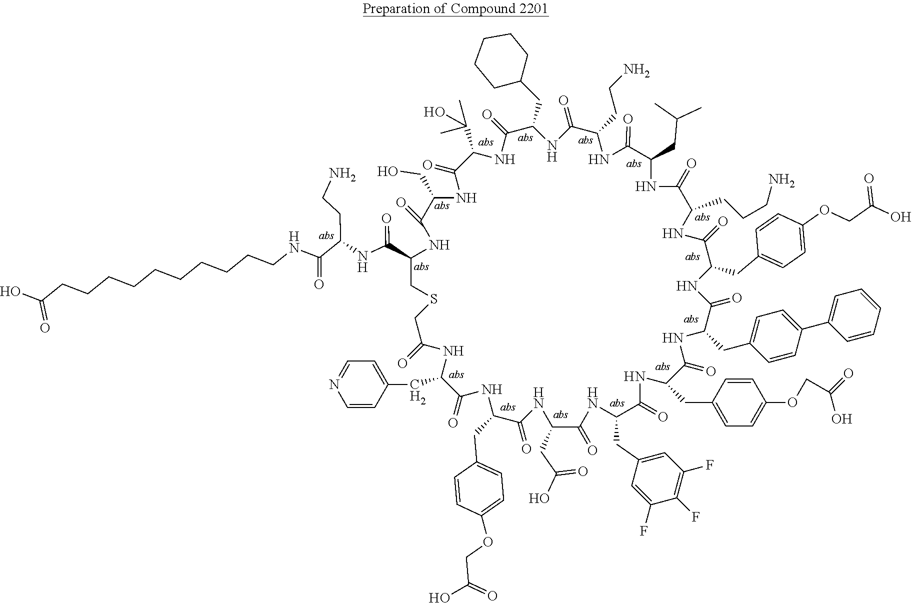 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cc(F)c(F)c(F)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 2201 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 2199 composed of the following general procedures: “Prelude Resin-swelling procedure”, “Prelude Single-coupling procedure”, or “Prelude Double-coupling procedure”, “Symphony X Single-coupling procedure”, “Prelude Single-Coupling Manual Addition Procedure A” was followed with Fmoc-Phe(3,4,5-triF)—OH, “Prelude Chloroacetic Anhydride coupling procedure”, “Prelude Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 17.6 mg, and its estimated purity by LCMS analysis was 94%. Analysis condition 1: Retention time=1.67 min; ESI-MS(+) m/z [M+2H]²⁺: 1240.1.

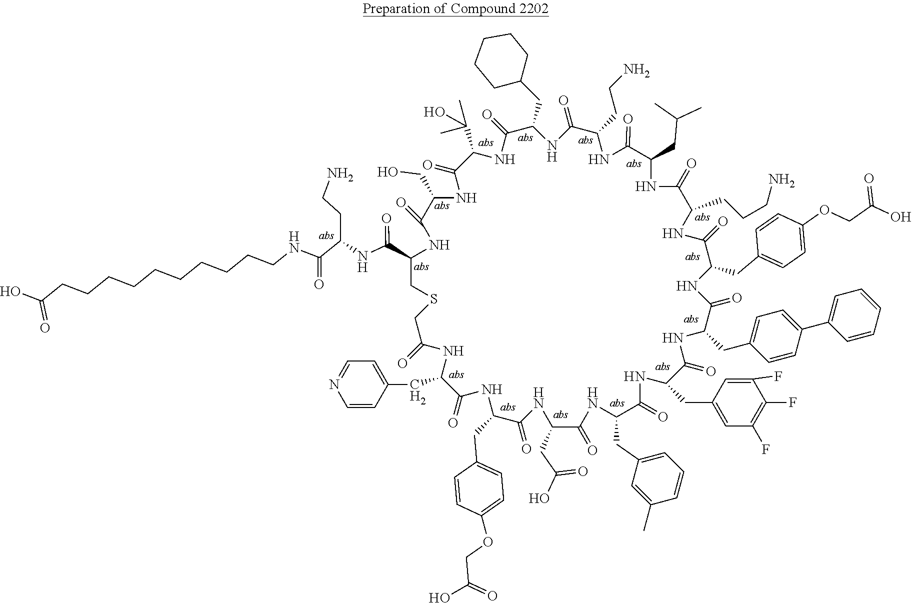 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3cc(F)c(F)c(F)c3)NC2=O)c1]

Compound 2202 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 2199 composed of the following general procedures: “Prelude Resin-swelling procedure”, “Prelude Single-coupling procedure”, or “Prelude Double-coupling procedure”, “Symphony X Single-coupling procedure”, “Prelude Single-Coupling Manual Addition Procedure A” was followed with Fmoc-Phe (3,4,5-triF)—OH, “Prelude Chloroacetic Anhydride coupling procedure”, “Prelude Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 31.6 mg, and its estimated purity by LCMS analysis was 96.4%. Analysis condition 2: Retention time=1.71 min; ESI-MS(+) m/z [M+2H]²⁺: 1210.3.

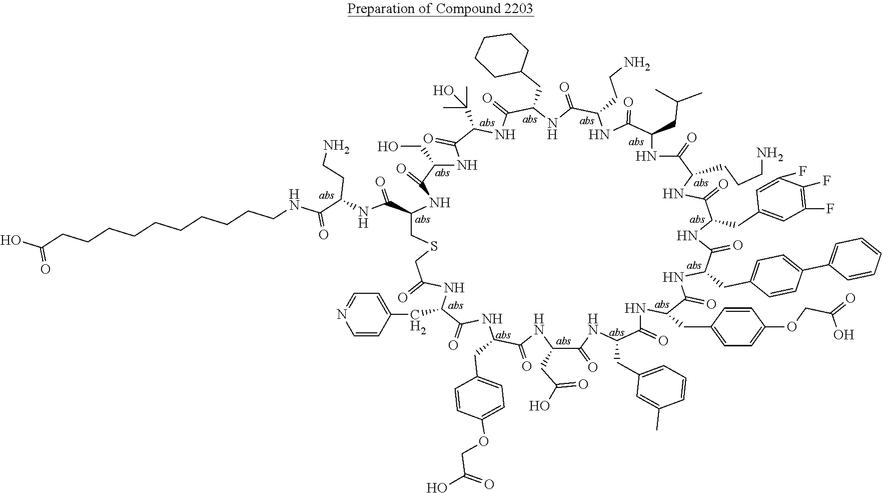

Compound 2203 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 2199 composed of the following general procedures: “Prelude Resin-swelling procedure”, “Prelude Single-coupling procedure”, or “Prelude Double-coupling procedure”, “Symphony X Single-coupling procedure”, “Prelude Single-Coupling Manual Addition Procedure A” was followed with Fmoc-Phe (3,4,5-triF)—OH, “Prelude Chloroacetic Anhydride coupling procedure”, “Prelude Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 19.2 mg, and its estimated purity by LCMS analysis was 92.8%. Analysis condition 2: Retention time=1.8 min; ESI-MS(+) m/z [M+2H]²⁺: 1210.2.

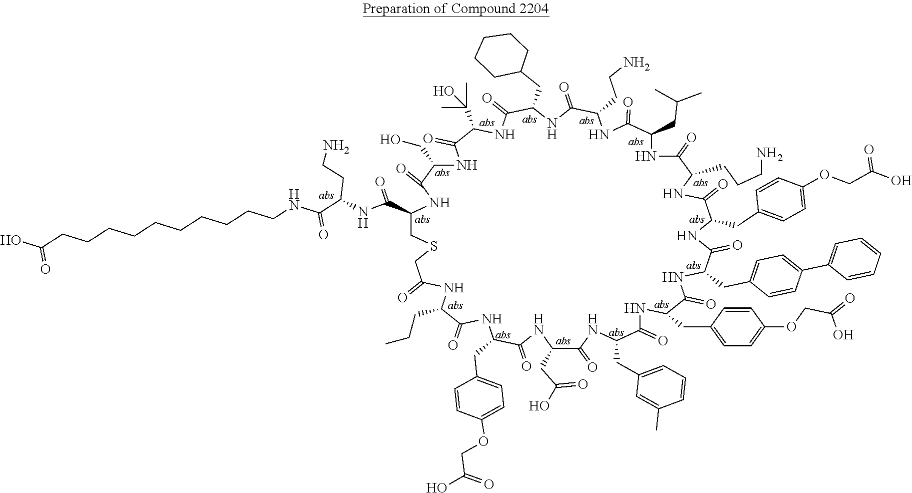 [SMILES: CCC[C@@H]1NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2204 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Nva-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 6.3 mg, and its estimated purity by LCMS analysis was 94.6%. Analysis condition 2: Retention time=1.74 min; ESI-MS(+) m/z [M+2H]²⁺: 1195.1.

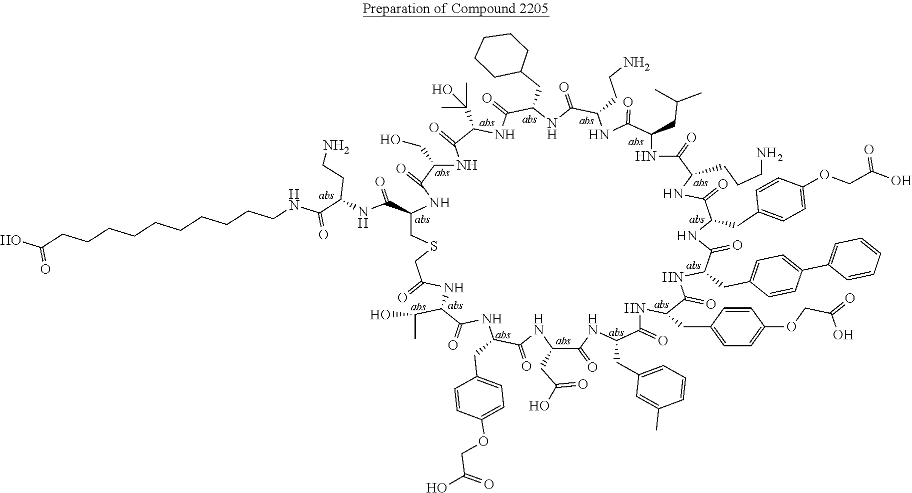 [SMILES: Cc1cccc(C[C@@H]2NC(=O)[C@H](CC(=O)O)NC(=O)[C@H](Cc3ccc(OCC(=O)O)cc3)NC(=O)[C@H]([C@H](C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2205 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.5 mg, and its estimated purity by LCMS analysis was 93.7%. Analysis condition 2: Retention time=1.52 min; ESI-MS(+) m/z [M+2H]²⁺: 798.1.

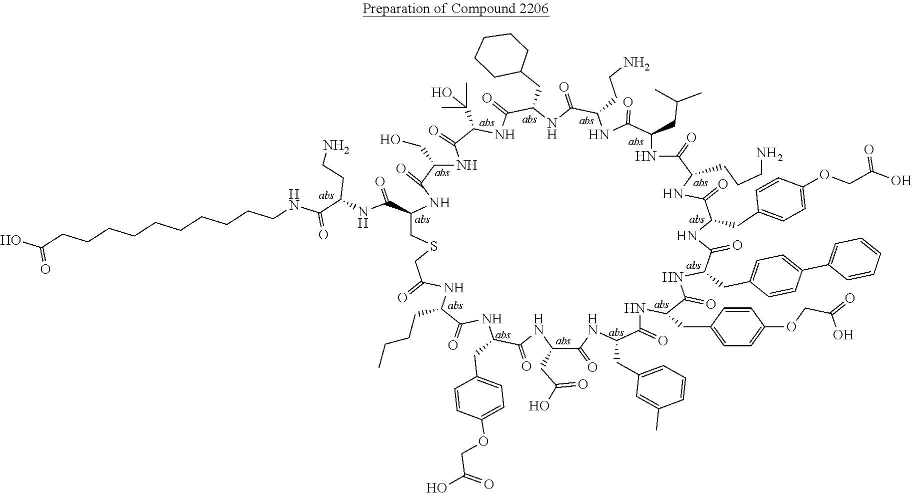 [SMILES: CCCC[C@@H]1NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2206 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-homo-Ser(Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 11% B, 11-51% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 9.5 mg, and its estimated purity by LCMS analysis was 98.2%. Analysis condition 2: Retention time=1.7 min; ESI-MS(+) m/z [M+2H]²⁺: 1203.9.

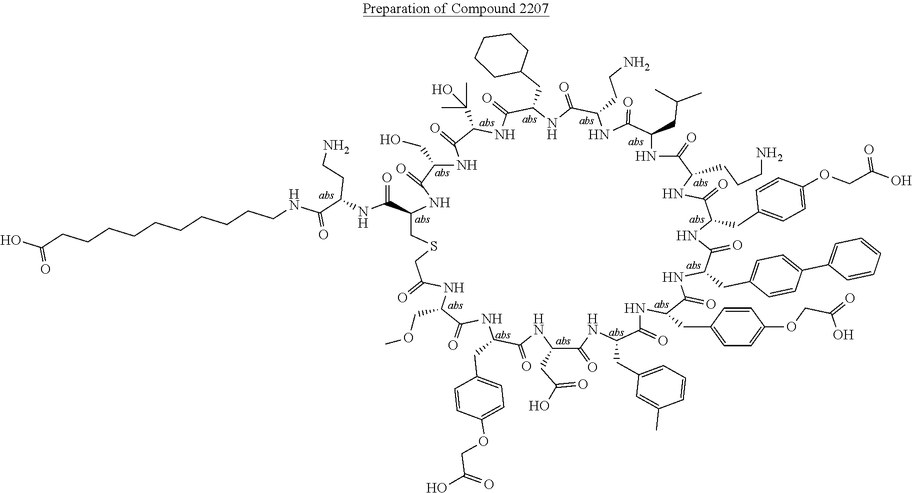 [SMILES: COC[C@@H]1NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2207 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Ser(Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.3 mg, and its estimated purity by LCMS analysis was 93.1%. Analysis condition 2: Retention time=1.53 min; ESI-MS(+) m/z [M+2H]²⁺: 1196.4.

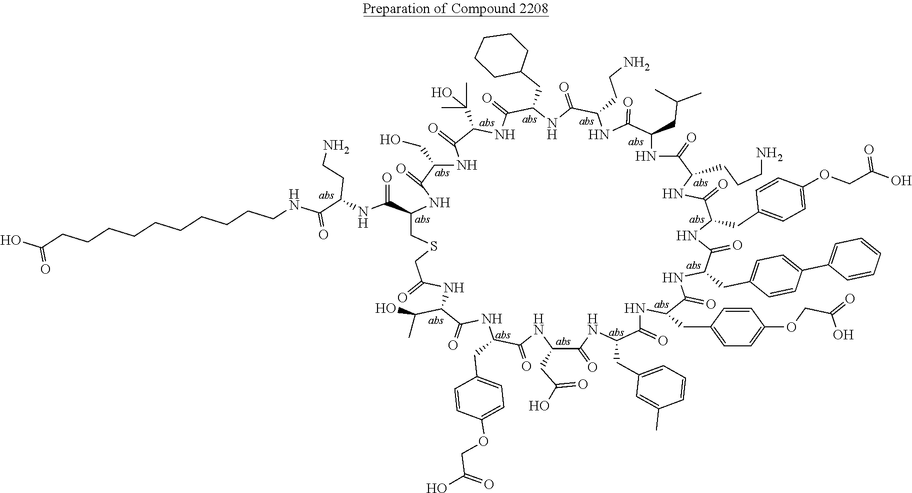 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2208 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-AlloThr(tBu)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 14.1 mg, and its estimated purity by LCMS analysis was 95.7%. Analysis condition 1: Retention time=1.43 min; ESI-MS(+) m/z [M+2H]²⁺: 1196.3.

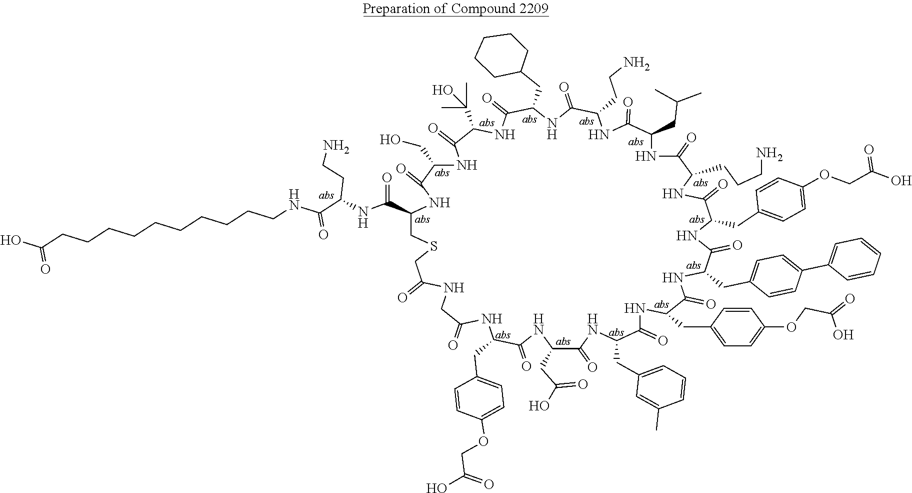 [SMILES: Cc1cccc(C[C@@H]2NC(=O)[C@H](CC(=O)O)NC(=O)[C@H](Cc3ccc(OCC(=O)O)cc3)NC(=O)CNC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2209 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 1-minute hold at 100% B; Flow Rate: 40 mL/min; Column Temperature: 25 C. Fraction collection was triggered by MS signals. Fractions containing the desired product were combined and dried via centrifugal evaporation. The yield of the product was 5.6 mg, and its estimated purity by LCMS analysis was 100%. Analysis condition 1: Retention time=1.64 min; ESI-MS(+) m/z [M+2H]²⁺: 1174.2.

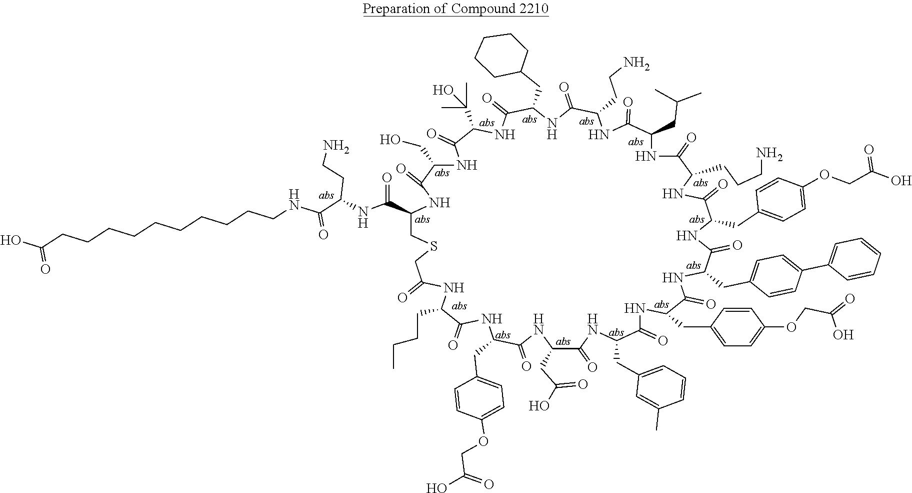 [SMILES: CCCC[C@@H]1NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2210 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Nle-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 5.4 mg, and its estimated purity by LCMS analysis was 100%. Analysis condition 1: Retention time=1.63 min; ESI-MS(+) m/z [M+2H]²⁺: 1202.9.

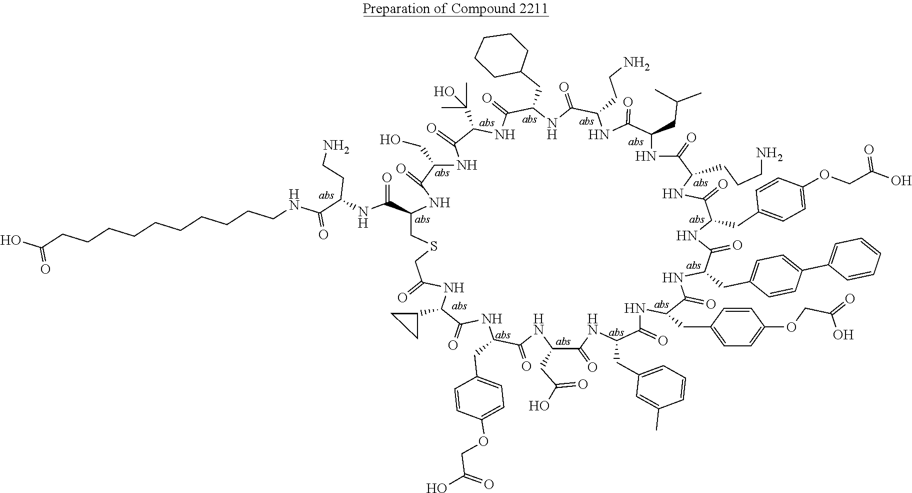 [SMILES: Cc1cccc(C[C@@H]2NC(=O)[C@H](CC(=O)O)NC(=O)[C@H](Cc3ccc(OCC(=O)O)cc3)NC(=O)[C@H](C3CC3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2211 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Gly (cyclopropyl)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 7 mg, and its estimated purity by LCMS analysis was 95.5%. Analysis condition 2: Retention time=1.54 min; ESI-MS(+) m/z [M+2H]²⁺: 1194.2.

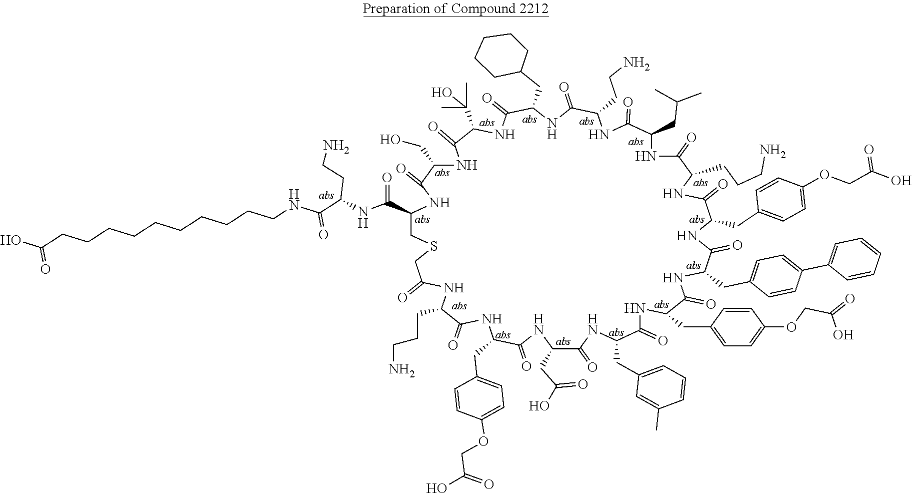 [SMILES: Cc1cccc(C[C@@H]2NC(=O)[C@H](CC(=O)O)NC(=O)[C@H](Cc3ccc(OCC(=O)O)cc3)NC(=O)[C@H](CCCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2212 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 2-minute hold at 100% B; Flow Rate: 45 mL/min; Column Temperature: 25 C. Fraction collection was triggered by MS signals. Fractions containing the desired product were combined and dried via centrifugal evaporation. The yield of the product was 12.3 mg, and its estimated purity by LCMS analysis was 92.9%. Analysis condition 2: Retention time=1.64 min; ESI-MS(+) m/z [M+2H]²⁺: 1203.

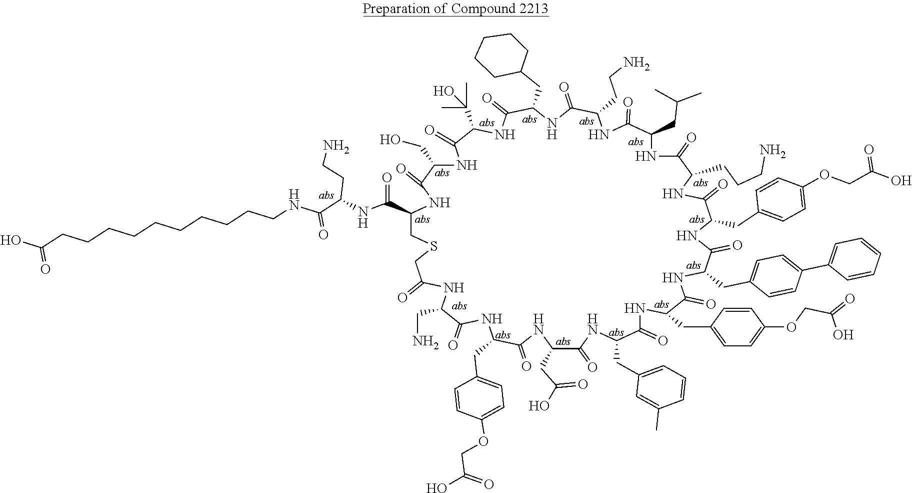 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2213 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 8.2 mg, and its estimated purity by LCMS analysis was 94.5%. Analysis condition 2: Retention time=1.59 min; ESI-MS(+) m/z [M+2H]²⁺: 1189.4.

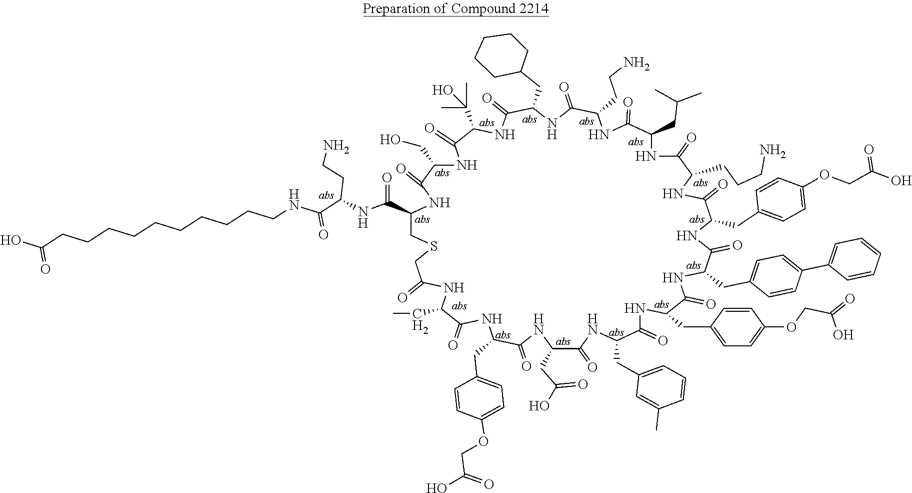 [SMILES: CC[C@@H]1NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2214 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Abu-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 8.4 mg, and its estimated purity by LCMS analysis was 95.6%. Analysis condition 2: Retention time=1.43 min; ESI-MS(+) m/z [M+2H]²⁺: 1188.3.

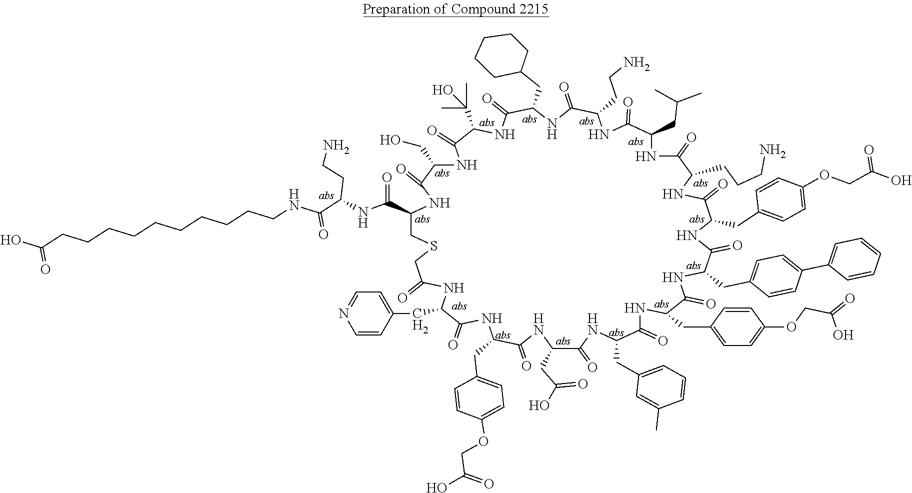 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2215 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Ala (2-pyridyl)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 17.3 mg, and its estimated purity by LCMS analysis was 91.3%. Analysis condition 2: Retention time=1.41 min; ESI-MS(+) m/z [M+2H]²⁺: 1220.2.

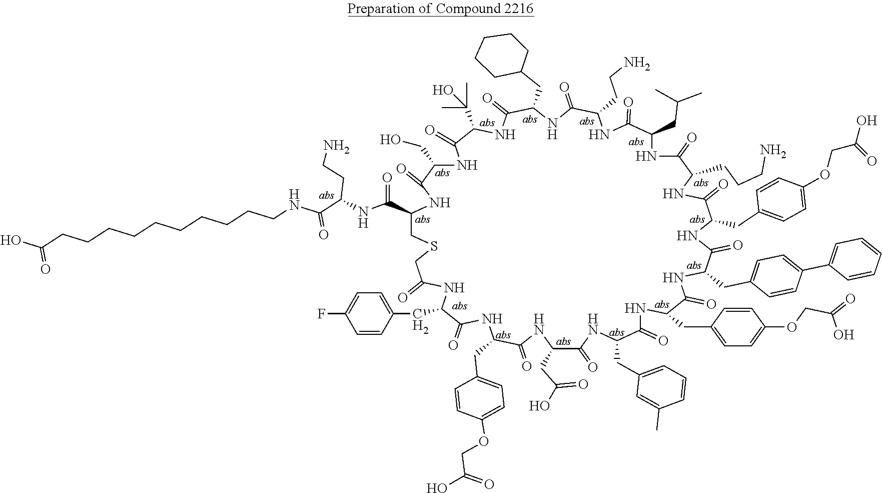 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2216 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe(4-F)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 19.3 mg, and its estimated purity by LCMS analysis was 95.2%. Analysis condition 2: Retention time=1.63 min; ESI-MS(+) m/z [M+2H]²⁺: 1228.4.

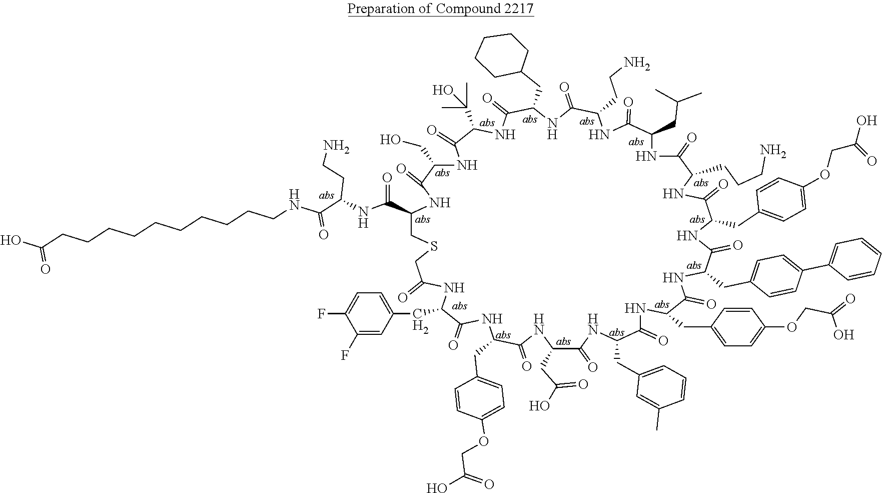 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)c(F)c3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2217 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (3,4-diF)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 23% B, 23-63% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 6.3 mg, and its estimated purity by LCMS analysis was 100%. Analysis condition 1: Retention time=1.67 min; ESI-MS(+) m/z [M+2H]²⁺: 1237.4.

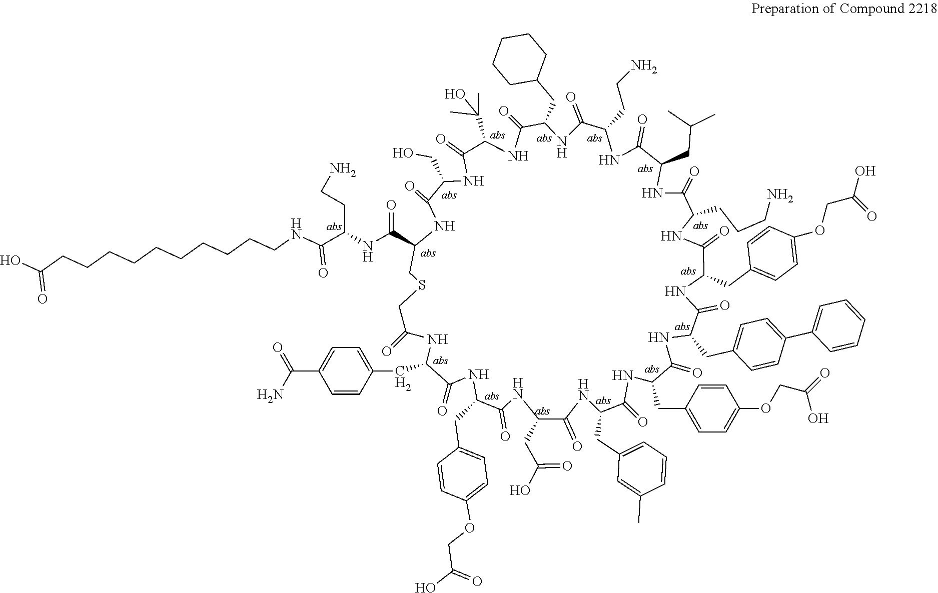 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2218 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (4-CONH₂)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 17% B, 17-57% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.3 mg, and its estimated purity by LCMS analysis was 93.7%. Analysis condition 2: Retention time=1.49 min; ESI-MS(+) m/z [M+2H]²⁺: 1241.2.

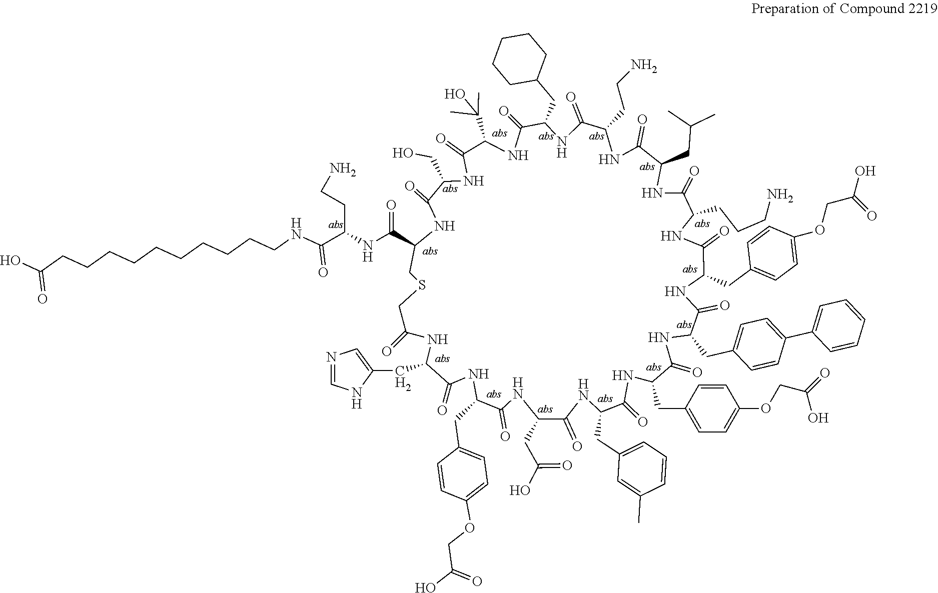 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cnc[nH]3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2219 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 20.3 mg, and its estimated purity by LCMS analysis was 92%. Analysis condition 2: Retention time=1.54 min; ESI-MS(+) m/z [M+3H]³⁺: 810.2.

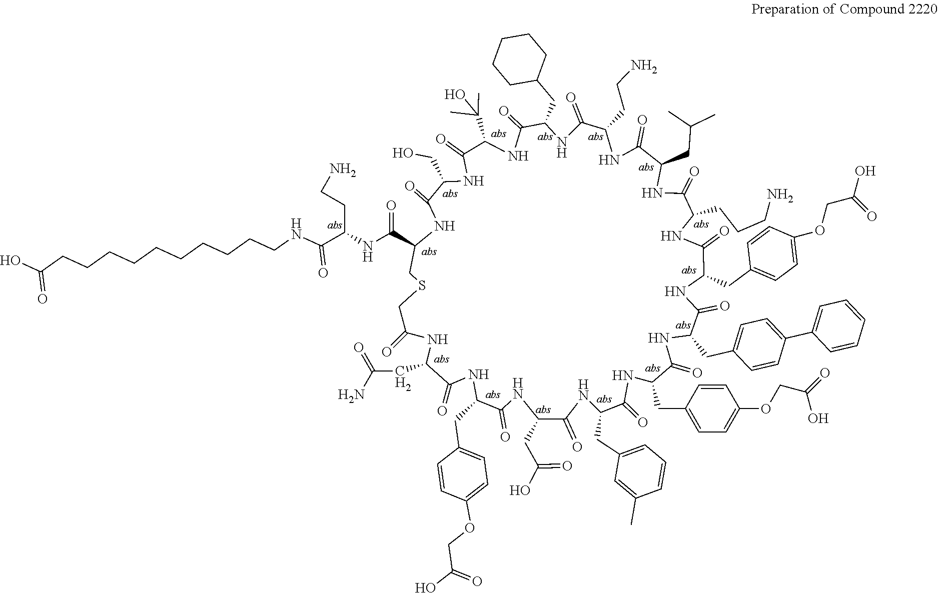 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2220 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 8.4 mg, and its estimated purity by LCMS analysis was 93.3%. Analysis condition 1: Retention time=1.66 min; ESI-MS(+) m/z [M+2H]²⁺: 1203.3.

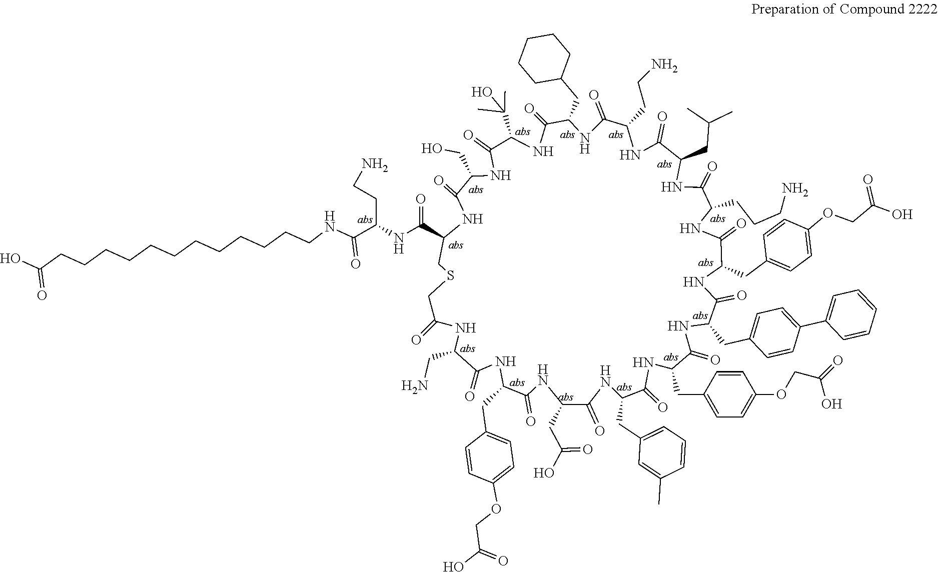 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2222 was prepared, using 2-Chlorotrityl resin pre-loaded with 13-((((9H-fluoren-9-yl) methoxy) carbonyl)amino)tridecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 3.9 mg, and its estimated purity by LCMS analysis was 97.7%. Analysis condition 1: Retention time=1.71 min; ESI-MS(+) m/z [M+2H]²⁺: 1203.1.

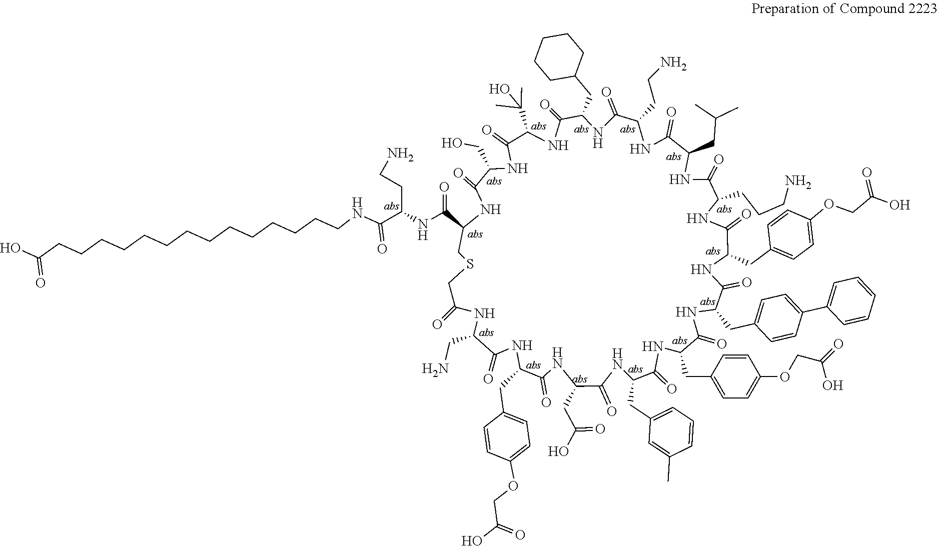 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2223 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 2.2 mg, and its estimated purity by LCMS analysis was 99.1%. Analysis condition 1: Retention time=1.65 min; ESI-MS(+) m/z [M+2H]²⁺: 1217.

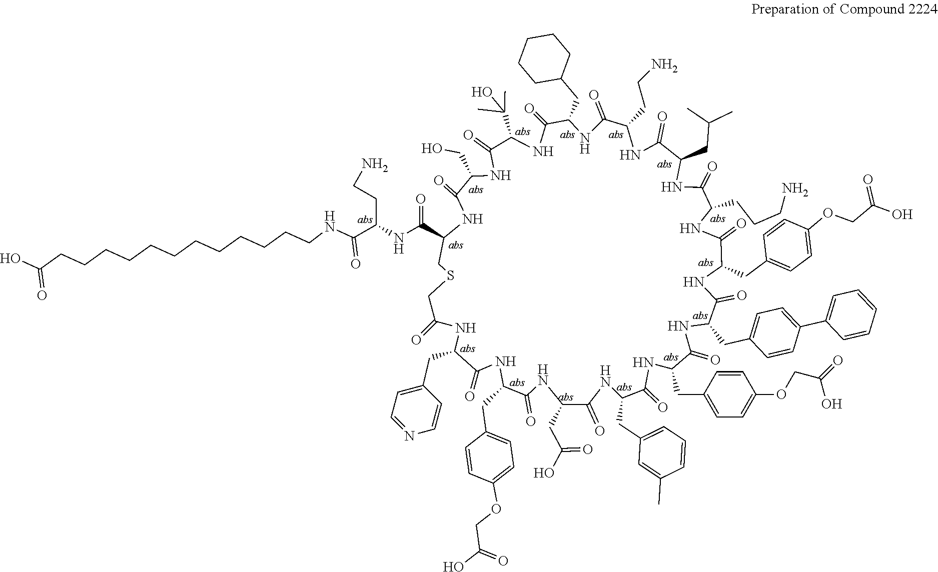 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2224 was prepared, using 2-Chlorotrityl resin pre-loaded with 13-((((9H-fluoren-9-yl) methoxy) carbonyl)amino)tridecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 6.5 mg, and its estimated purity by LCMS analysis was 100%. Analysis condition 2: Retention time=1.51 min; ESI-MS(+) m/z [M+2H]²⁺: 1234.2.

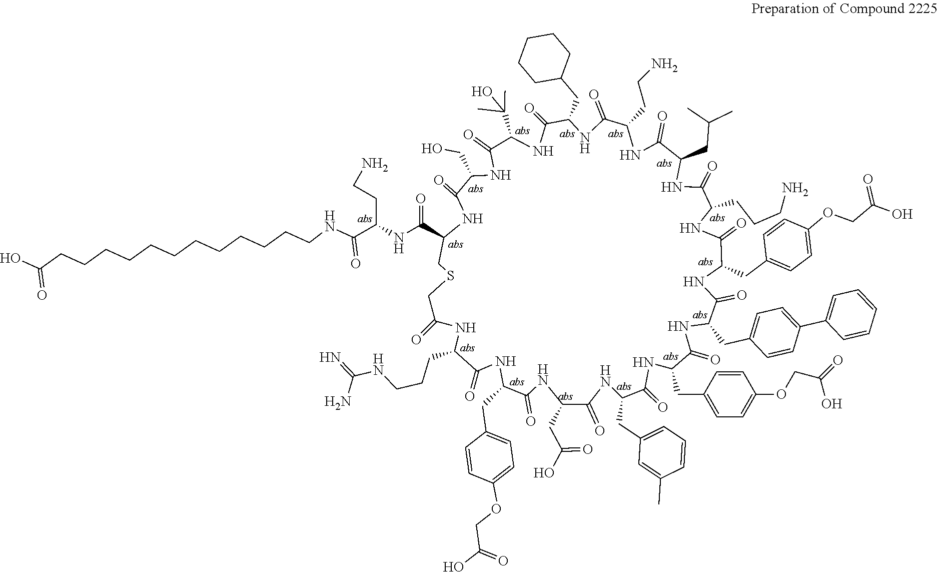 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2225 was prepared, using 2-Chlorotrityl resin pre-loaded with 13-((((9H-fluoren-9-yl) methoxy) carbonyl)amino)tridecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 11.8 mg, and its estimated purity by LCMS analysis was 91.2%. Analysis condition 1: Retention time=1.71 min; ESI-MS(+) m/z [M+2H]²⁺: 826.

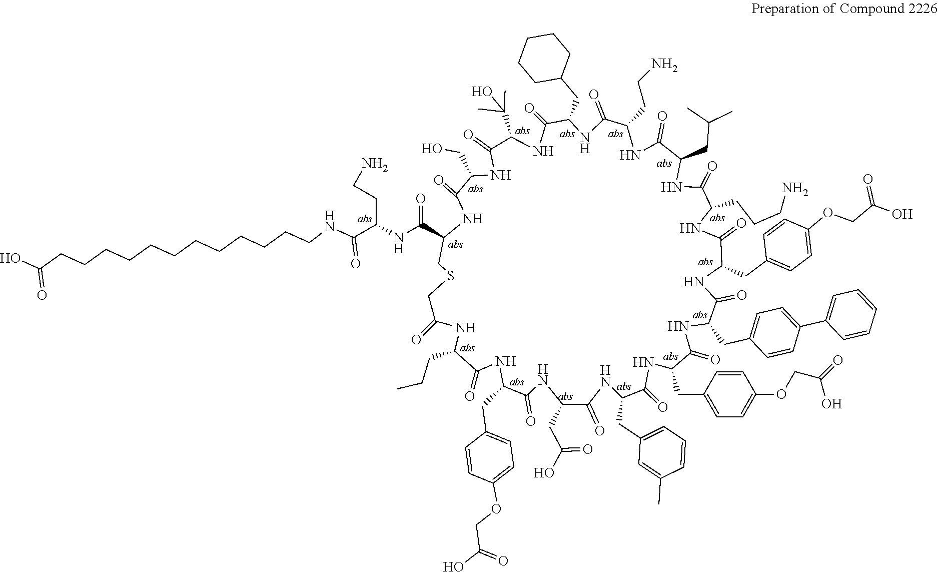 [SMILES: CCC[C@@H]1NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2226 was prepared, using 2-Chlorotrityl resin pre-loaded with 13-((((9H-fluoren-9-yl) methoxy) carbonyl)amino)tridecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-□-Nva-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 26% B, 26-66% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 6.6 mg, and its estimated purity by LCMS analysis was 100%. Analysis condition: Retention time=1.54 min; ESI-MS(+) m/z [M+2H]²⁺: 1209.

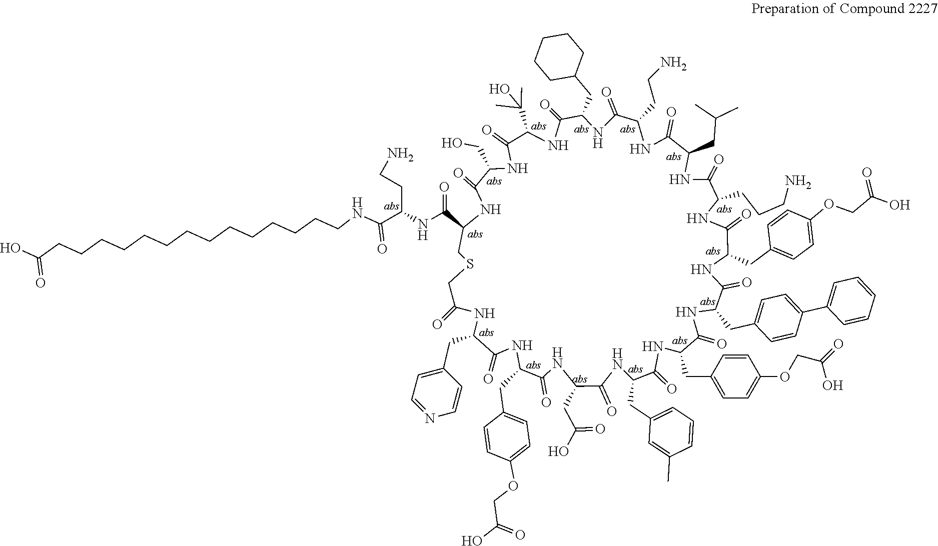 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2227 was prepared, using 2-Chlorotrityl resin pre-loaded with 15-((((9H-fluoren-9-yl) methoxy) carbonyl)amino) pentadecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 6.2 mg, and its estimated purity by LCMS analysis was 91.2%. Analysis condition 2: Retention time=1.57 min; ESI-MS(+) m/z [M+2H]²⁺: 1248.1.

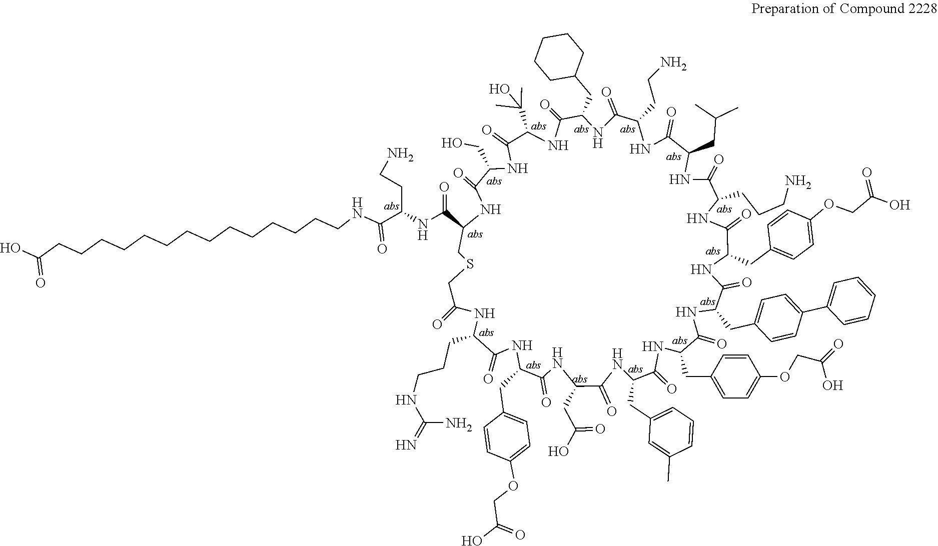 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2228 was prepared, using 2-Chlorotrityl resin pre-loaded with 15-((((9H-fluoren-9-yl) methoxy) carbonyl)amino) pentadecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-□-Nva-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 7.1 mg, and its estimated purity by LCMS analysis was 83.7%. Analysis condition 1: Retention time=1.7 min; ESI-MS(+) m/z [M+2H]²⁺: 1252.1.

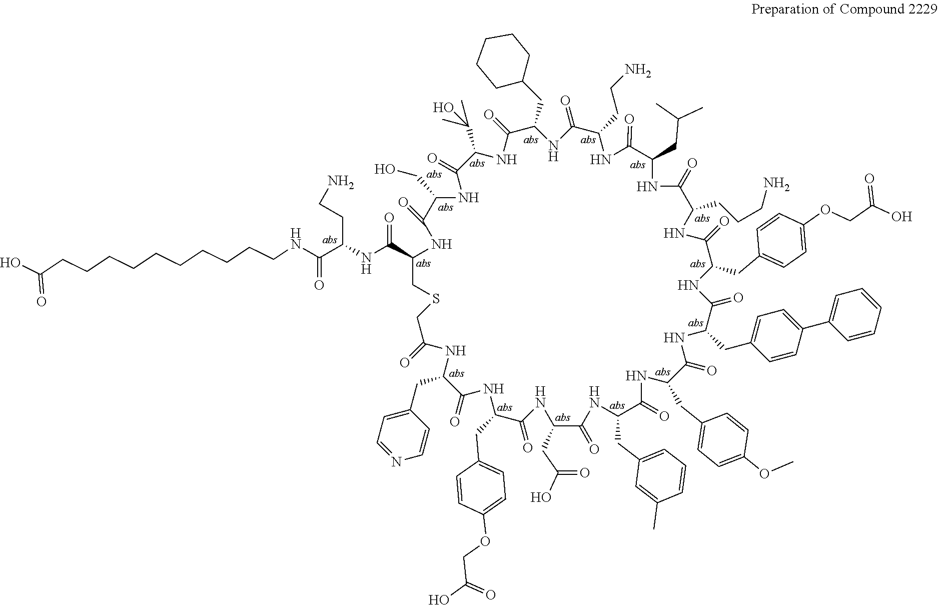 [SMILES: COc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 2229 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of composed of the following general procedures: Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Tyr(Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 12.8 mg, and its estimated purity by LCMS analysis was 96.3%. Analysis condition 1: Retention time=1.61 min; ESI-MS(+) m/z [M+3H]³⁺: 798.9.

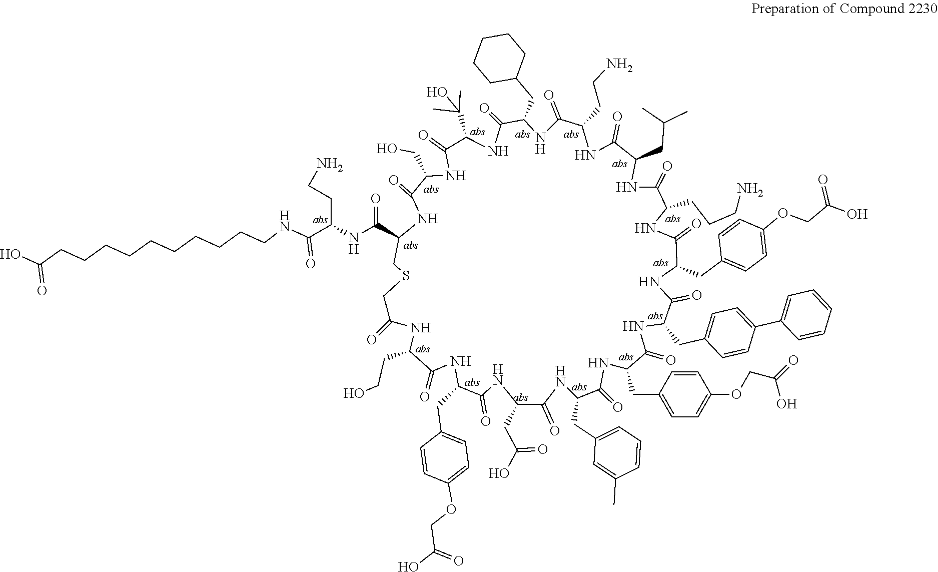 [SMILES: Cc1cccc(C[C@@H]2NC(=O)[C@H](CC(=O)O)NC(=O)[C@H](Cc3ccc(OCC(=O)O)cc3)NC(=O)[C@H](CCO)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2230 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-homo-Ser(tBu)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 2.6 mg, and its estimated purity by LCMS analysis was 97.6%. Analysis condition 1: Retention time=1.74 min; ESI-MS(+) m/z [M+2H]²⁺: 1196.4.

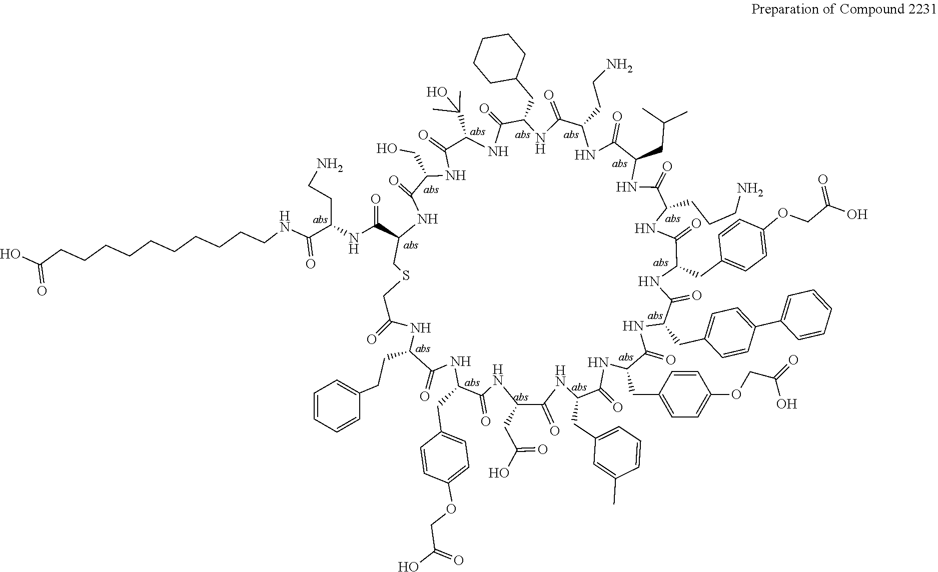 [SMILES: Cc1cccc(C[C@@H]2NC(=O)[C@H](CC(=O)O)NC(=O)[C@H](Cc3ccc(OCC(=O)O)cc3)NC(=O)[C@H](CCc3ccccc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2231 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-□-homo-Phe-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 24% B, 24-64% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 1.8 mg, and its estimated purity by LCMS analysis was 93.4%. Analysis condition 1: Retention time=1.82 min; ESI-MS(+) m/z [M+2H]²⁺: 1226.2.

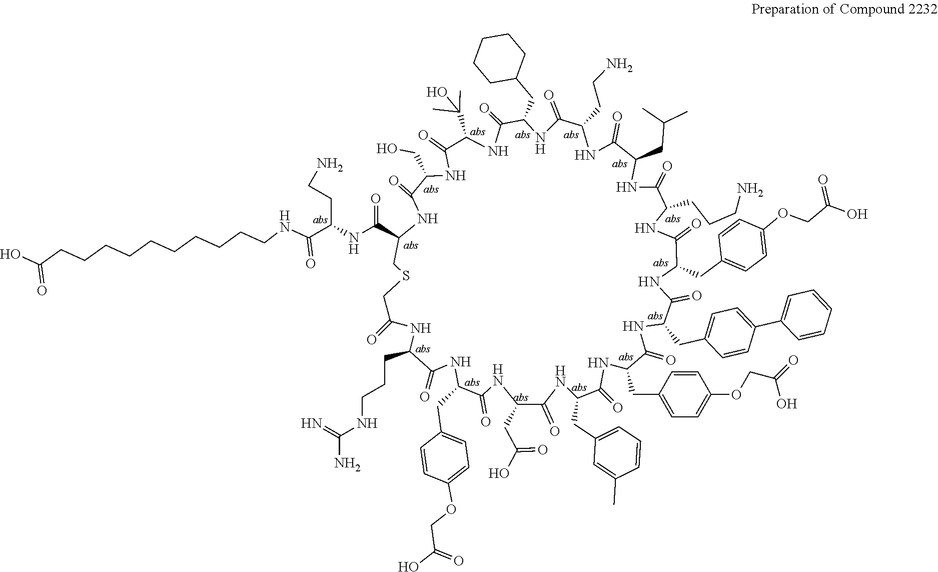 [SMILES: Cc1cccc(C[C@@H]2NC(=O)[C@H](CC(=O)O)NC(=O)[C@H](Cc3ccc(OCC(=O)O)cc3)NC(=O)[C@@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2232 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-D-Arg(Pbf)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.1 mg, and its estimated purity by LCMS analysis was 84.5%. Analysis condition 1: Retention time=1.63 min; ESI-MS(+) m/z [M+2H]²⁺: 1224.3.

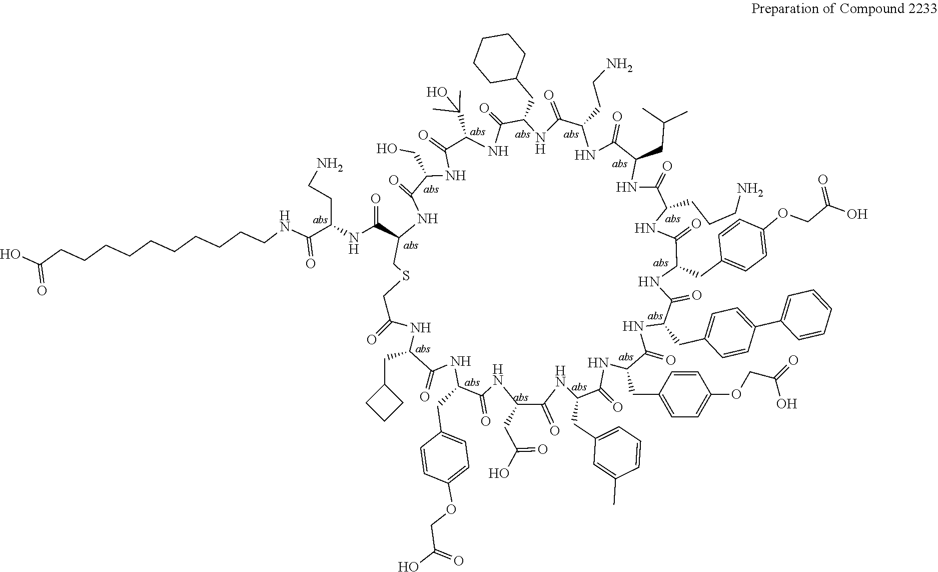 [SMILES: Cc1cccc(C[C@@H]2NC(=O)[C@H](CC(=O)O)NC(=O)[C@H](Cc3ccc(OCC(=O)O)cc3)NC(=O)[C@H](CC3CCC3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2233 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-□-Ala (cyclobutyl)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 10.7 mg, and its estimated purity by LCMS analysis was 99.3%. Analysis condition 1: Retention time=1.72 min; ESI-MS(+) m/z [M+2H]²⁺: 806.1.

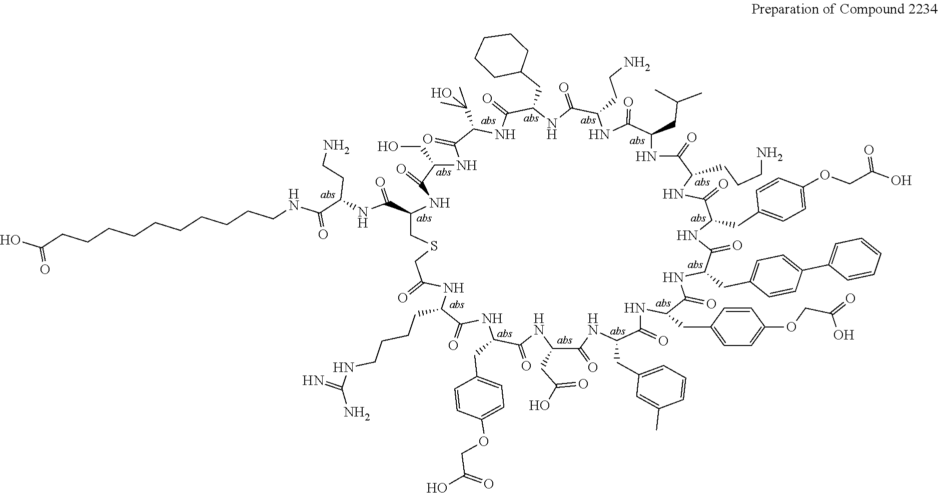

Compound 2234 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-homo-Arg(Pbf)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 2.1 mg, and its estimated purity by LCMS analysis was 92%. Analysis condition 2: Retention time=1.45 min; ESI-MS(+) m/z [M+2H]²⁺: 1231.1.

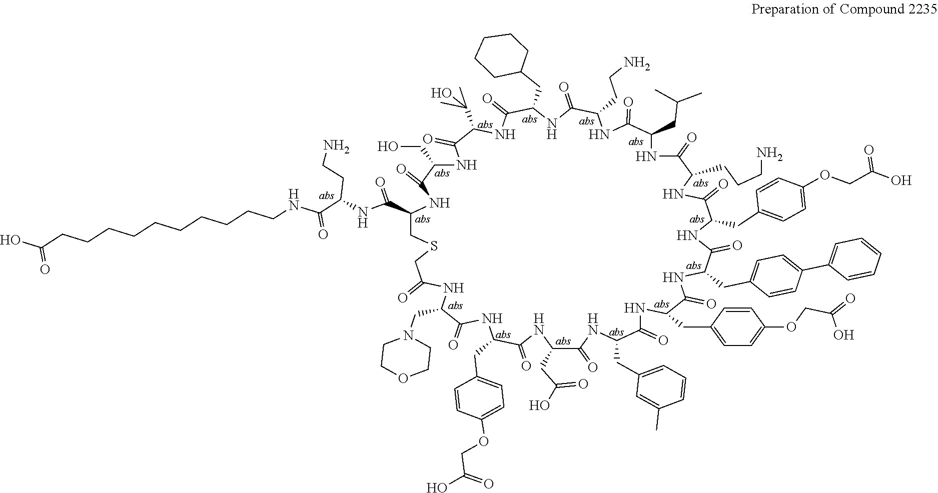

Compound 2235 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with(S)-2-((((9H-fluoren-9-yl) methoxy) carbonyl)amino)-3-morpholinopropanoic acid, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 3.4 mg, and its estimated purity by LCMS analysis was 94.6%. Analysis condition 2: Retention time=1.44 min; ESI-MS(+) m/z [M+2H]²⁺: 1224.1.

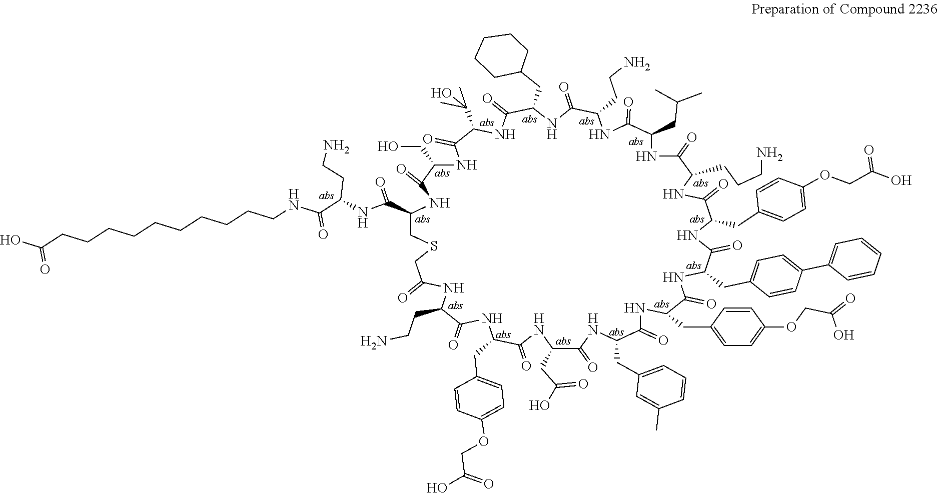

Compound 2236 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-D-Dab(Boc)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 10.1 mg, and its estimated purity by LCMS analysis was 95.5%. Analysis condition 1: Retention time=1.48 min; ESI-MS(+) m/z [M+2H]²⁺: 1196.3.

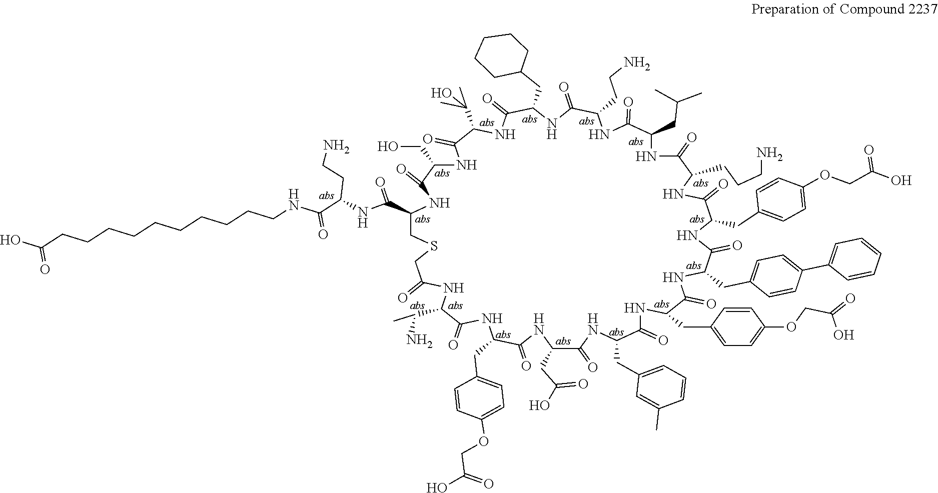

Compound 2237 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Dap(Boc, (S)-□-Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 24 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 2.7 mg, and its estimated purity by LCMS analysis was 100%. Analysis condition 1: Retention time=1.48 min; ESI-MS(+) m/z [M+2H]²⁺: 1196.1.

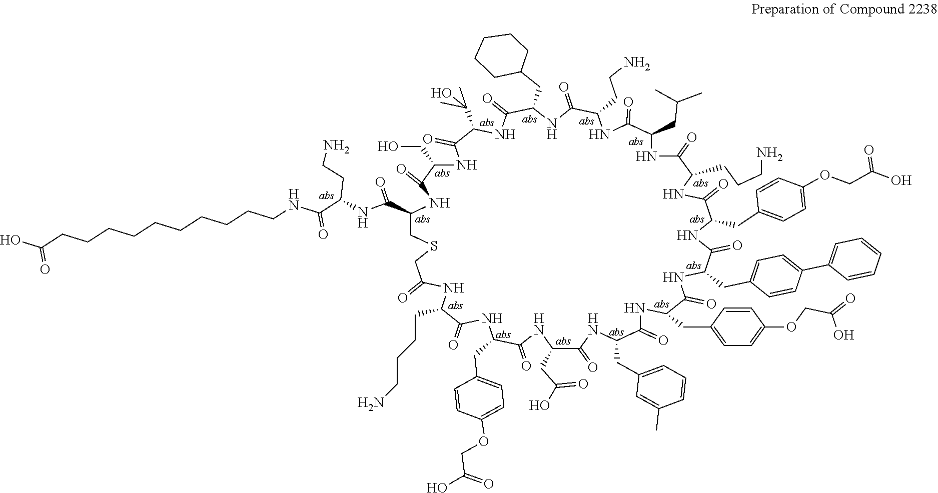

Compound 2238 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 1.3 mg, and its estimated purity by LCMS analysis was 100%. Analysis condition 2: Retention time=1.62 min; ESI-MS(+) m/z [M+2H]²⁺: 807.2.

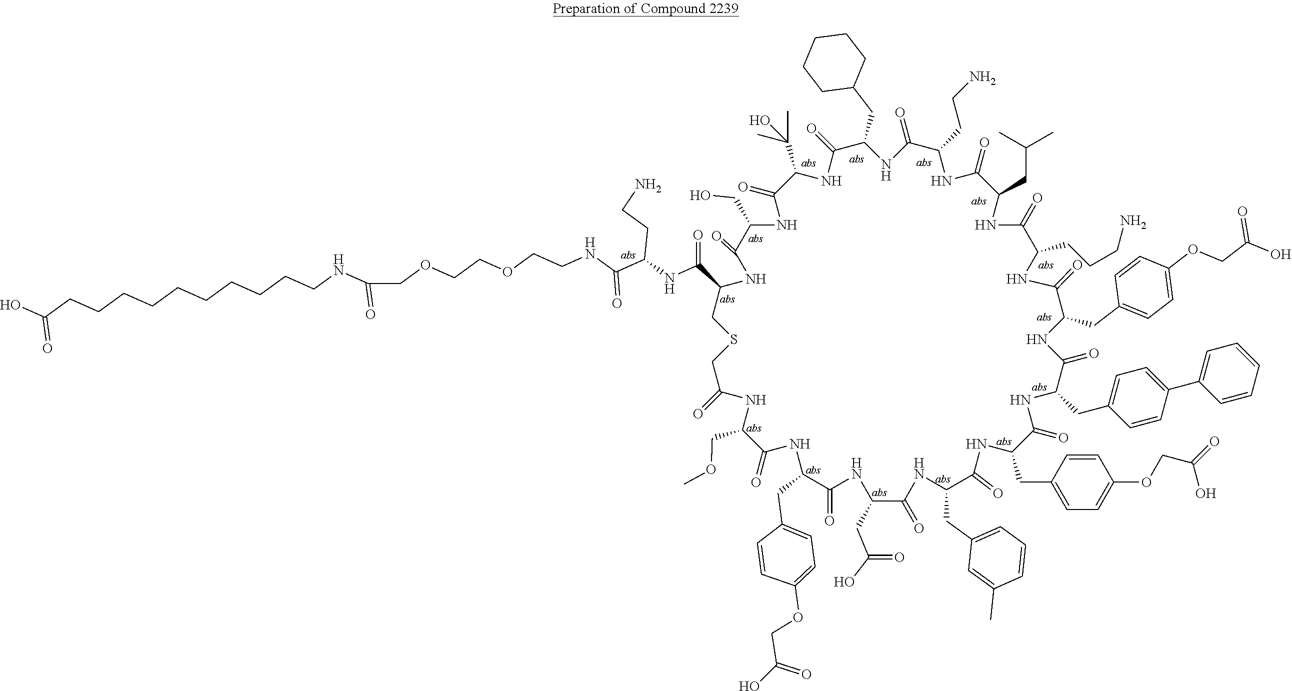 [SMILES: COC[C@@H]1NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2239 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Ser(Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 8 mg, and its estimated purity by LCMS analysis was 95.5%. Analysis condition 1: Retention time=1.78 min; ESI-MS(+) m/z [M+2H]²⁺: 1269.2.

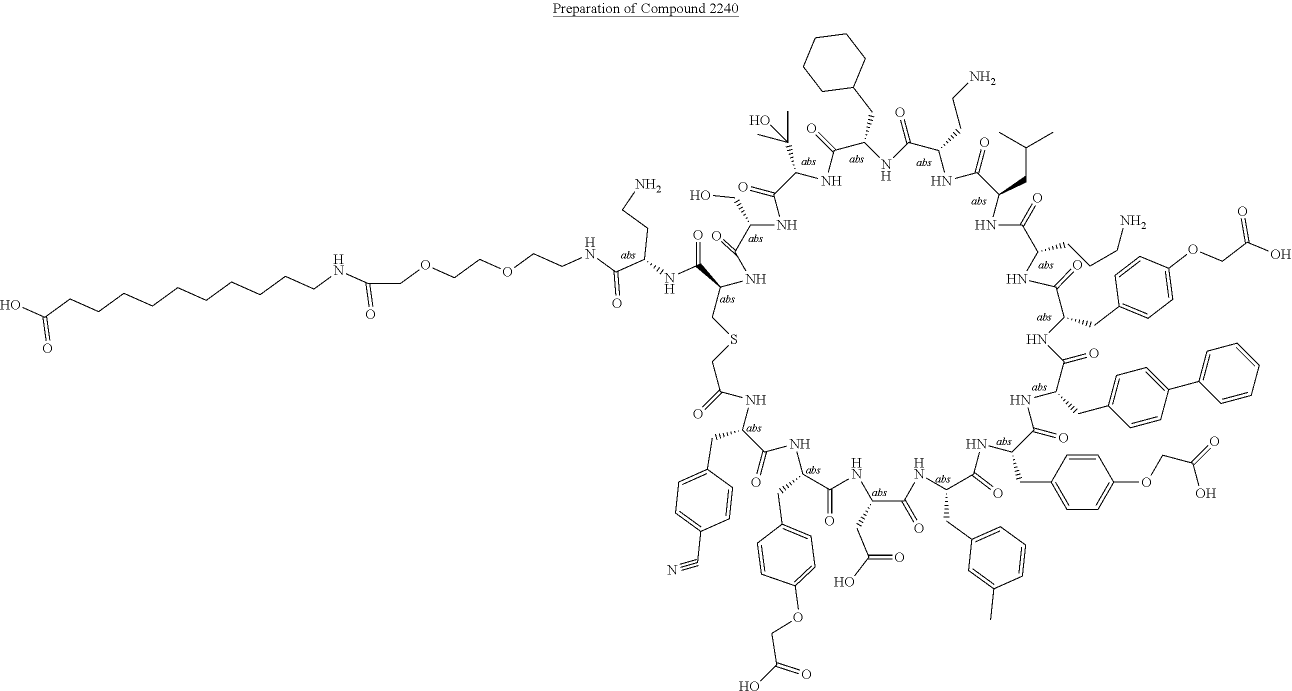 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C#N)cc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2240 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe (4-CN)—OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 24 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 6.7 mg, and its estimated purity by LCMS analysis was 95.8%. Analysis condition 1: Retention time=1.71 min; ESI-MS(+) m/z [M+2H]²⁺: 870.

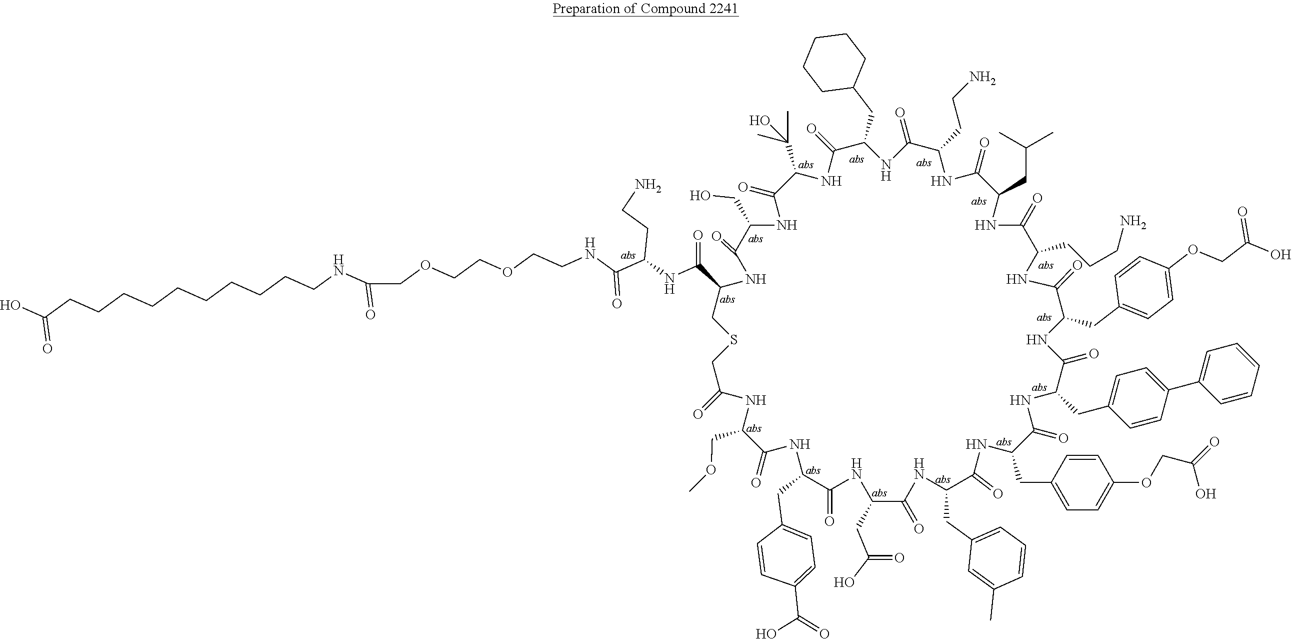 [SMILES: COC[C@@H]1NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Compound 2241 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Ser(Me)-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 9 mg, and its estimated purity by LCMS analysis was 91.1%. Analysis condition 1: Retention time=1.78 min; ESI-MS(+) m/z [M+2H]²⁺: 1254.2.

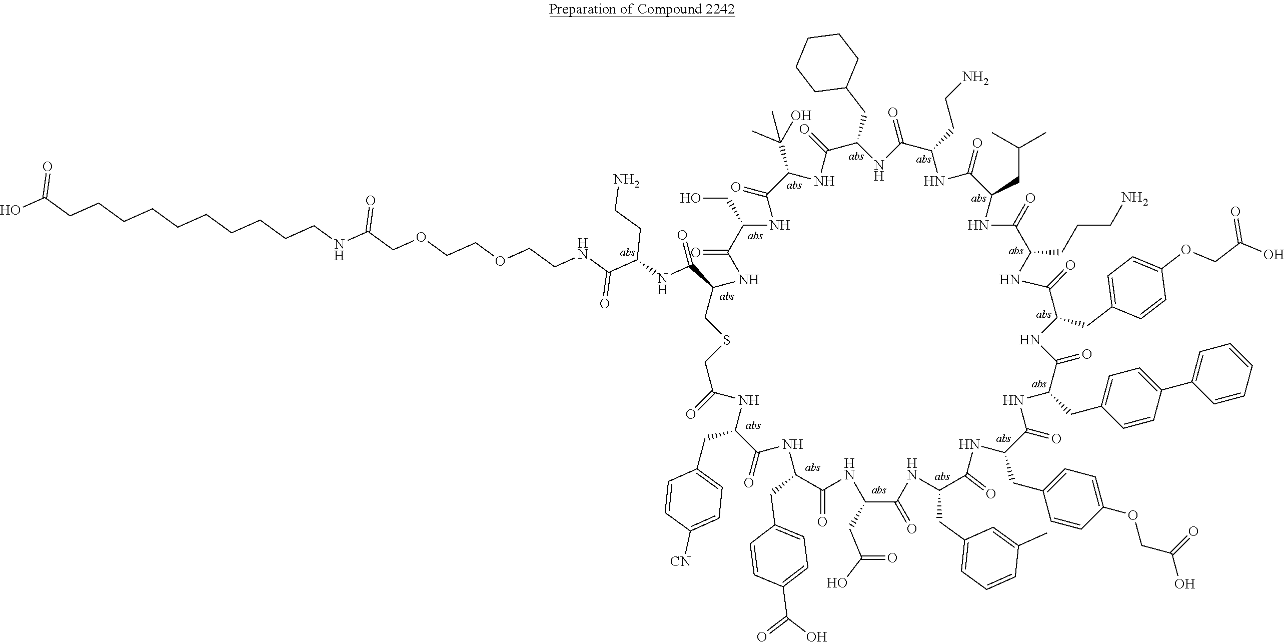

Compound 2242 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Phe(4-CN)—OH “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 16.4 mg, and its estimated purity by LCMS analysis was 100%. Analysis condition 2: Retention time=1.51 min; ESI-MS(+) m/z [M+3H]³⁺: 860.

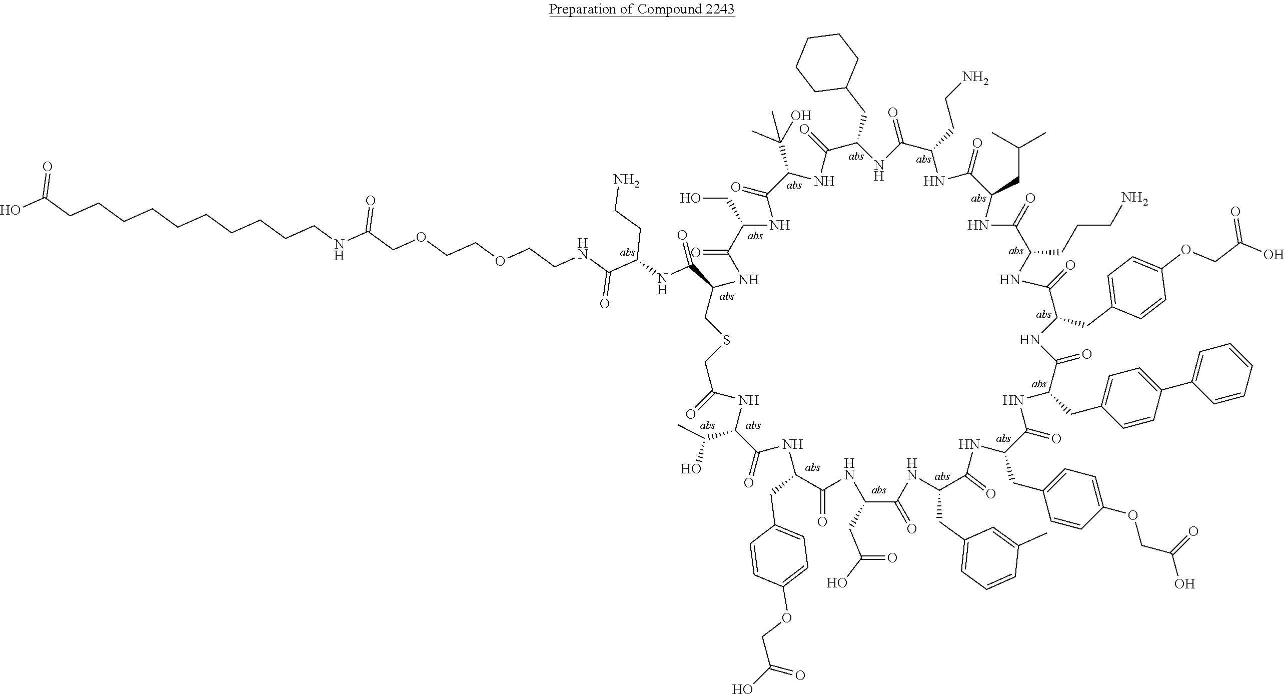 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2243 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 6.8 mg, and its estimated purity by LCMS analysis was 96.1%. Analysis condition 1: Retention time=1.7 min; ESI-MS(+) m/z [M+2H]²⁺: 1269.2.

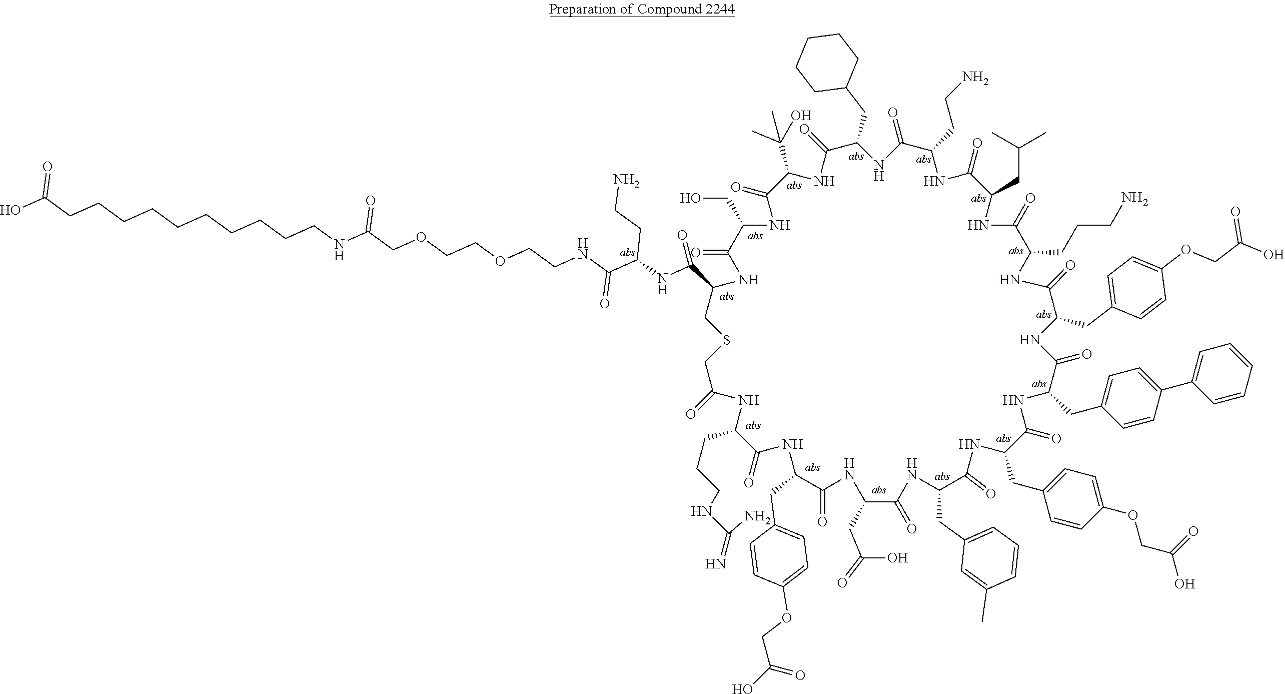 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2244 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 9.2 mg, and its estimated purity by LCMS analysis was 91.9%. Analysis condition 1: Retention time=1.67, 1.70 min; ESI-MS(+) m/z [M+3H]³⁺: 864.

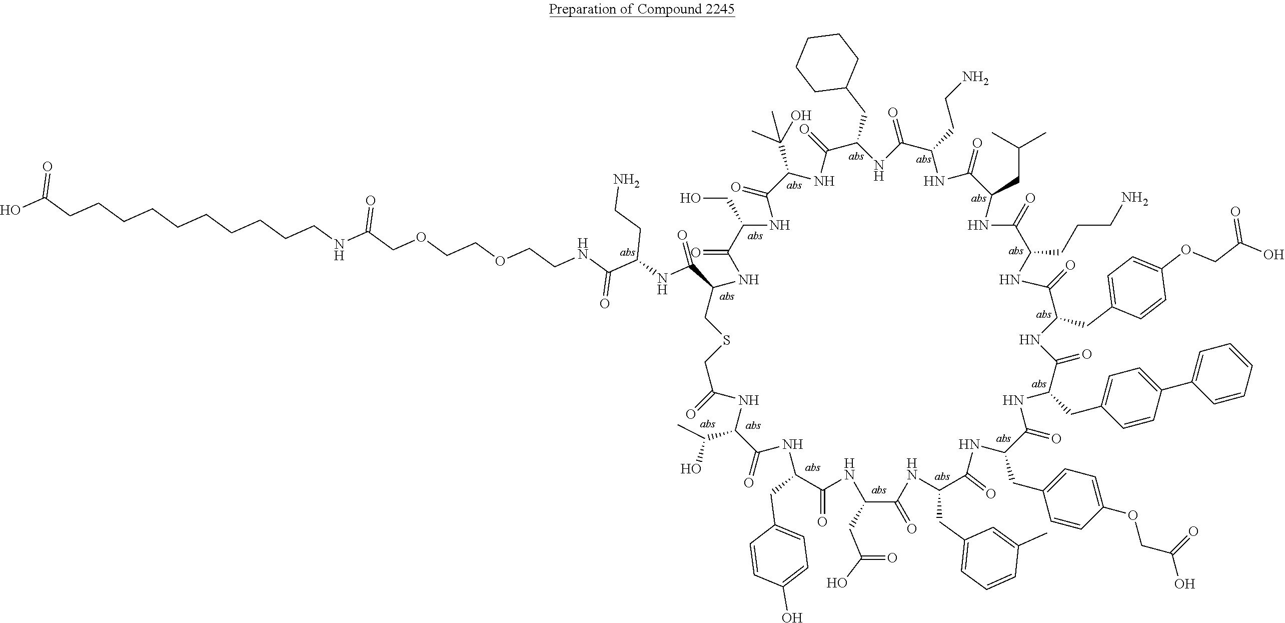 [SMILES: Cc1cccc(C[C@@H]2NC(=O)[C@H](CC(=O)O)NC(=O)[C@H](Cc3ccc(O)cc3)NC(=O)[C@H]([C@@H](C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2245 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 9.7 mg, and its estimated purity by LCMS analysis was 95.2%. Analysis condition 2: Retention time=1.62 min; ESI-MS(+) m/z [M+2H]²⁺: 1240.3.

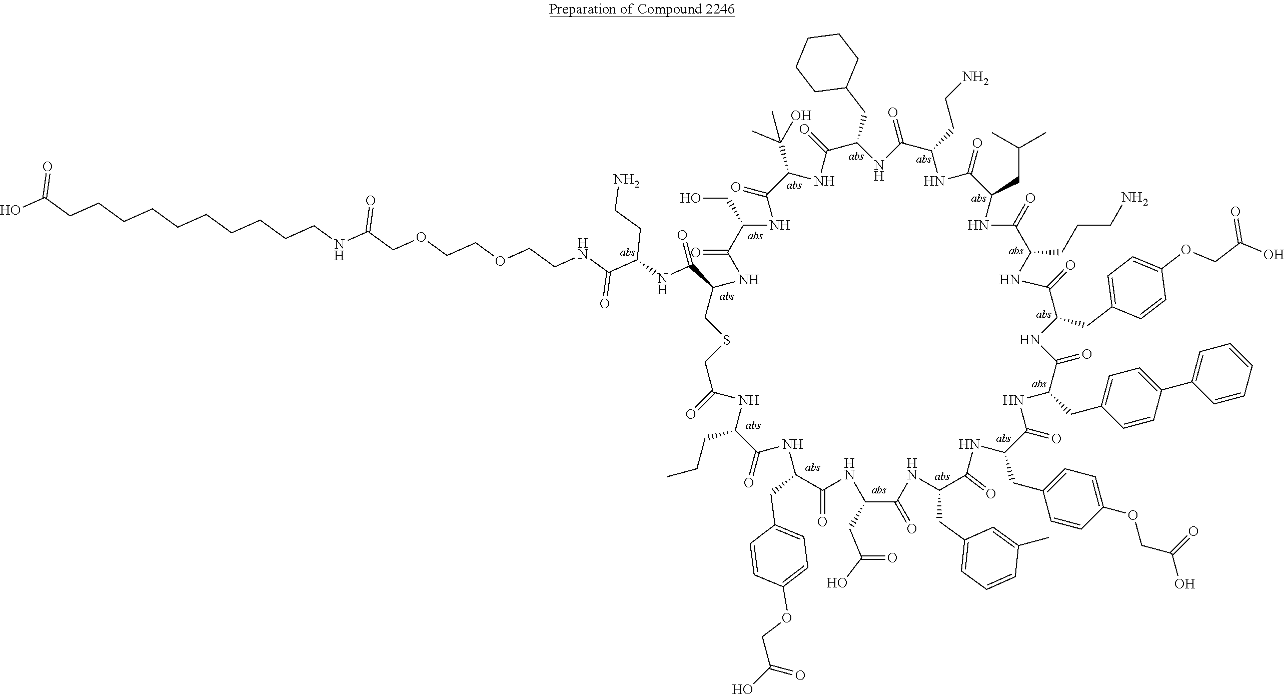 [SMILES: CCC[C@@H]1NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2246 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Nva-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 7.5 mg, and its estimated purity by LCMS analysis was 95.8%. Analysis condition 1: Retention time=1.81 min; ESI-MS(+) m/z [M+2H]²⁺: 1268.

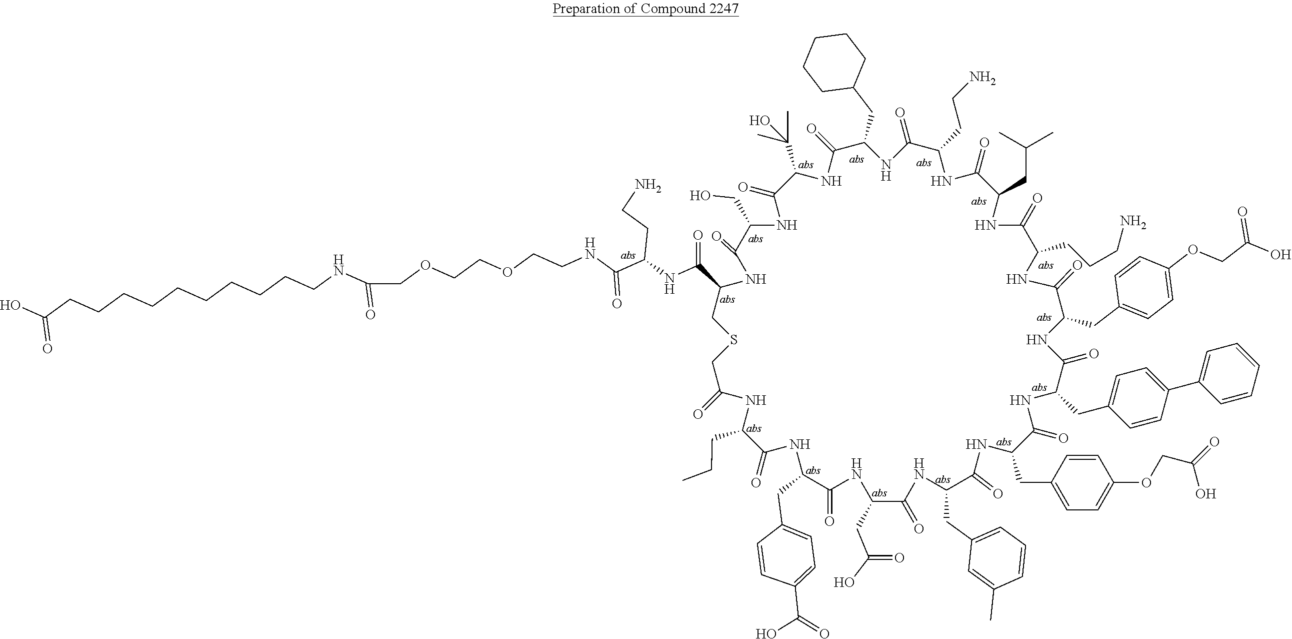 [SMILES: CCC[C@@H]1NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Compound 2247 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Nva-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 9.9 mg, and its estimated purity by LCMS analysis was 98.2%. Analysis condition 2: Retention time=1.75 min; ESI-MS(+) m/z [M+2H]²⁺: 1253.

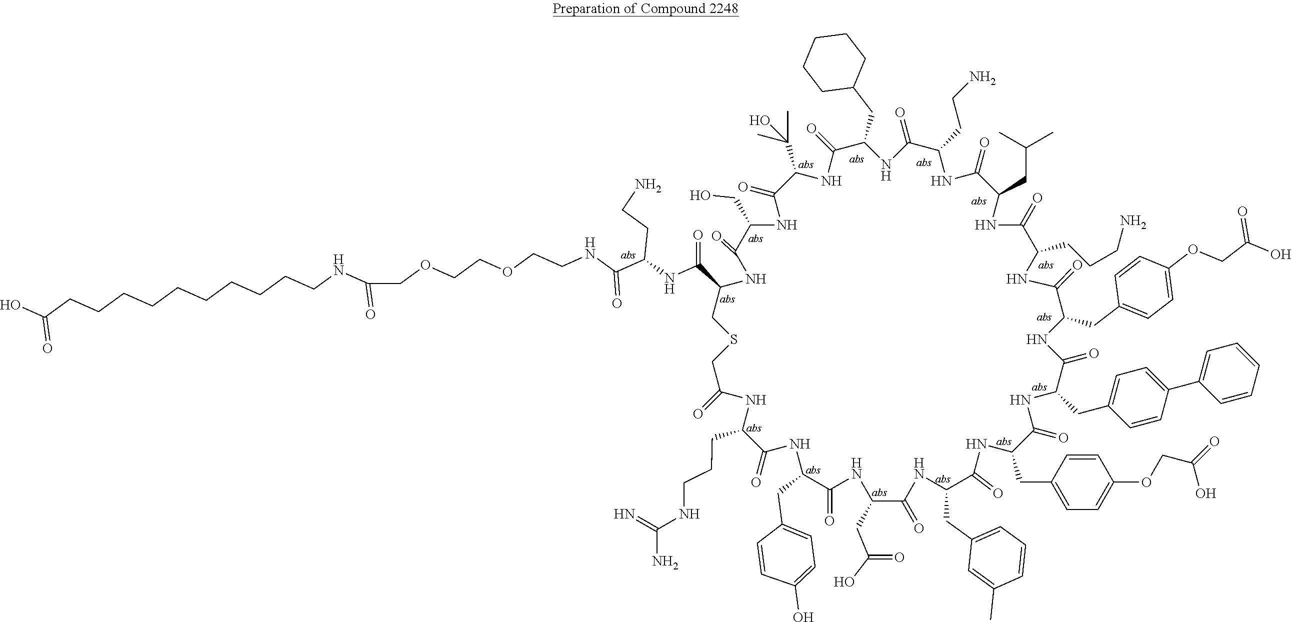 [SMILES: Cc1cccc(C[C@@H]2NC(=O)[C@H](CC(=O)O)NC(=O)[C@H](Cc3ccc(O)cc3)NC(=O)[C@H](CCCNC(=N)N)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2248 was prepared, using 2-Chlorotrityl resin pre-loaded with 11-((((9H-fluoren-9-yl) methoxy) carbonyl)amino) undecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The yield of the product was mg, and its estimated purity by LCMS analysis was %. Analysis condition: Retention time=1.63 min; ESI-MS(+) m/z [M+2H]²⁺:

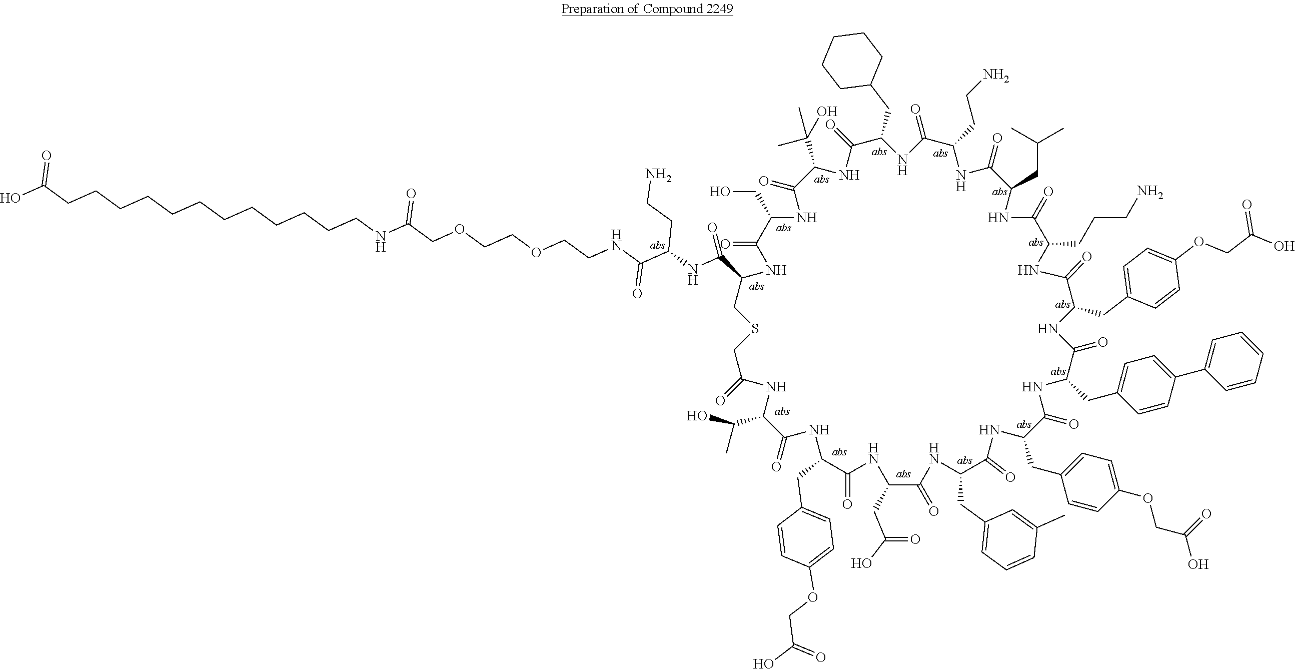 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2249 was prepared, using 2-Chlorotrityl resin pre-loaded with 13-((((9H-fluoren-9-yl) methoxy) carbonyl)amino)tridecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 24% B, 24-64% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9 mg, and its estimated purity by LCMS analysis was 90%. Analysis condition 2: Retention time=1.63 min; ESI-MS(+) m/z [M+2H]²⁺: 1283.2.

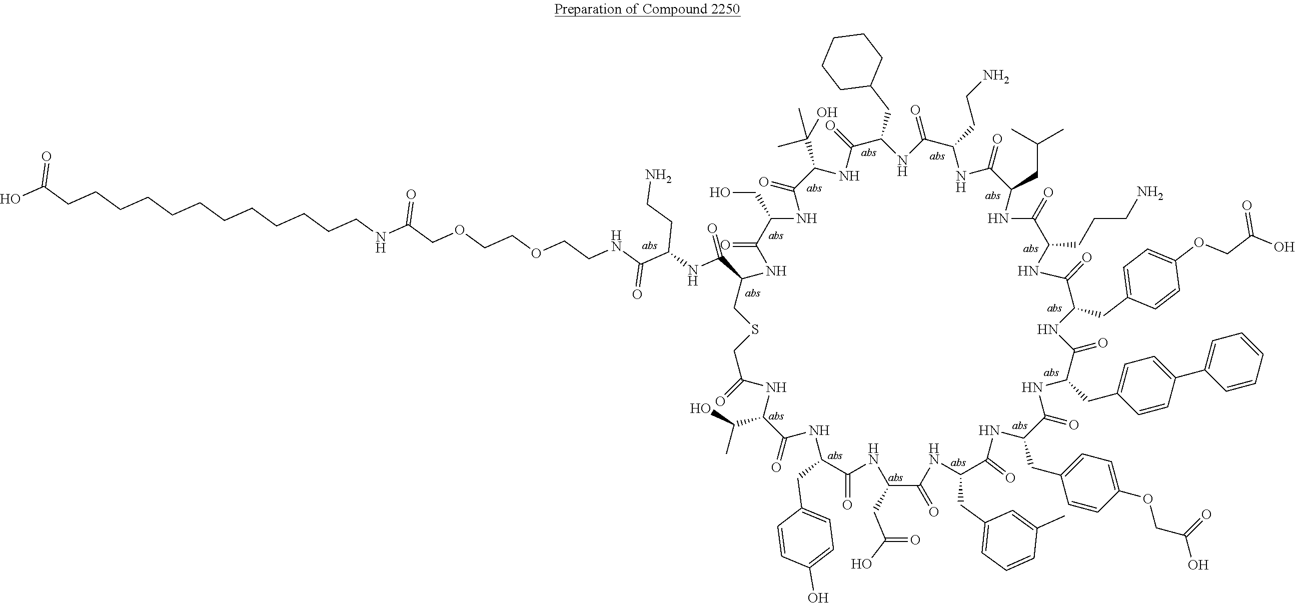 [SMILES: Cc1cccc(C[C@@H]2NC(=O)[C@H](CC(=O)O)NC(=O)[C@H](Cc3ccc(O)cc3)NC(=O)[C@H]([C@@H](C)O)NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2250 was prepared, using 2-Chlorotrityl resin pre-loaded with 13-((((9H-fluoren-9-yl) methoxy) carbonyl)amino)tridecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 24% B, 24-64% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 12.6 mg, and its estimated purity by LCMS analysis was 95.9%. Analysis condition 2: Retention time=1.66 min; ESI-MS(+) m/z [M+2H]²⁺: 1254.

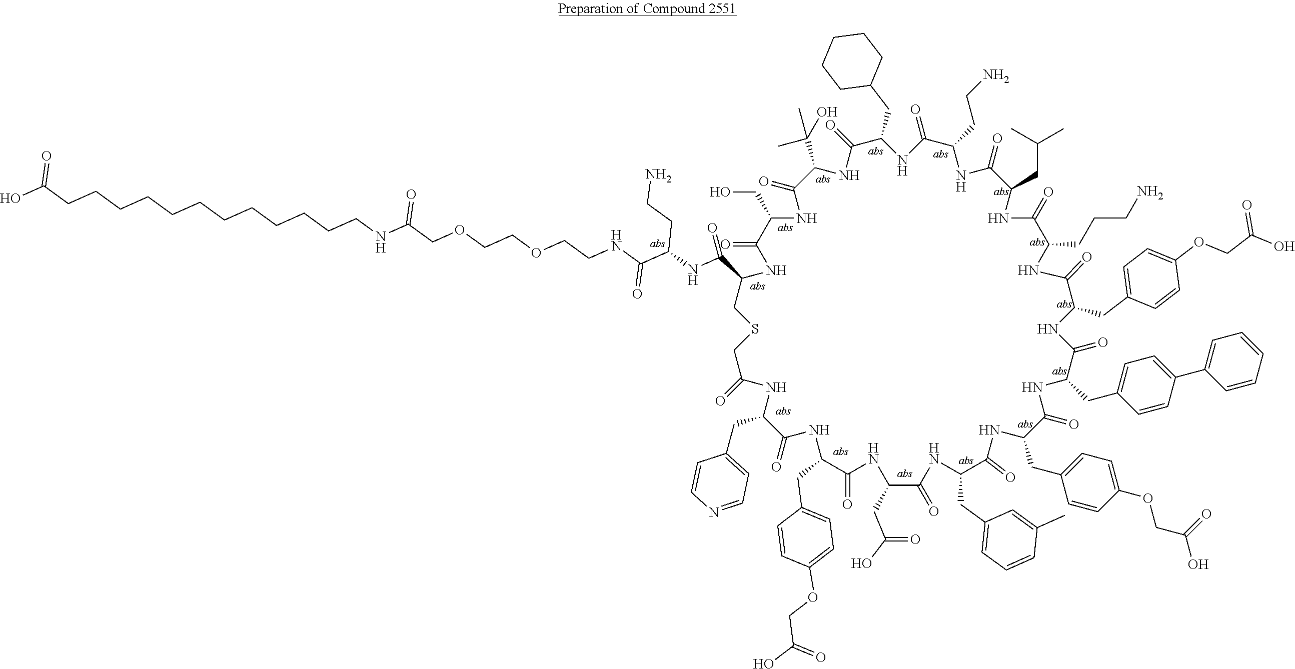 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2251 was prepared, using 2-Chlorotrityl resin pre-loaded with 13-((((9H-fluoren-9-yl) methoxy) carbonyl)amino)tridecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 9.6 mg, and its estimated purity by LCMS analysis was 95.5%. Analysis condition 1: Retention time=1.75 min; ESI-MS(+) m/z [M+3H]³⁺: 871.2.

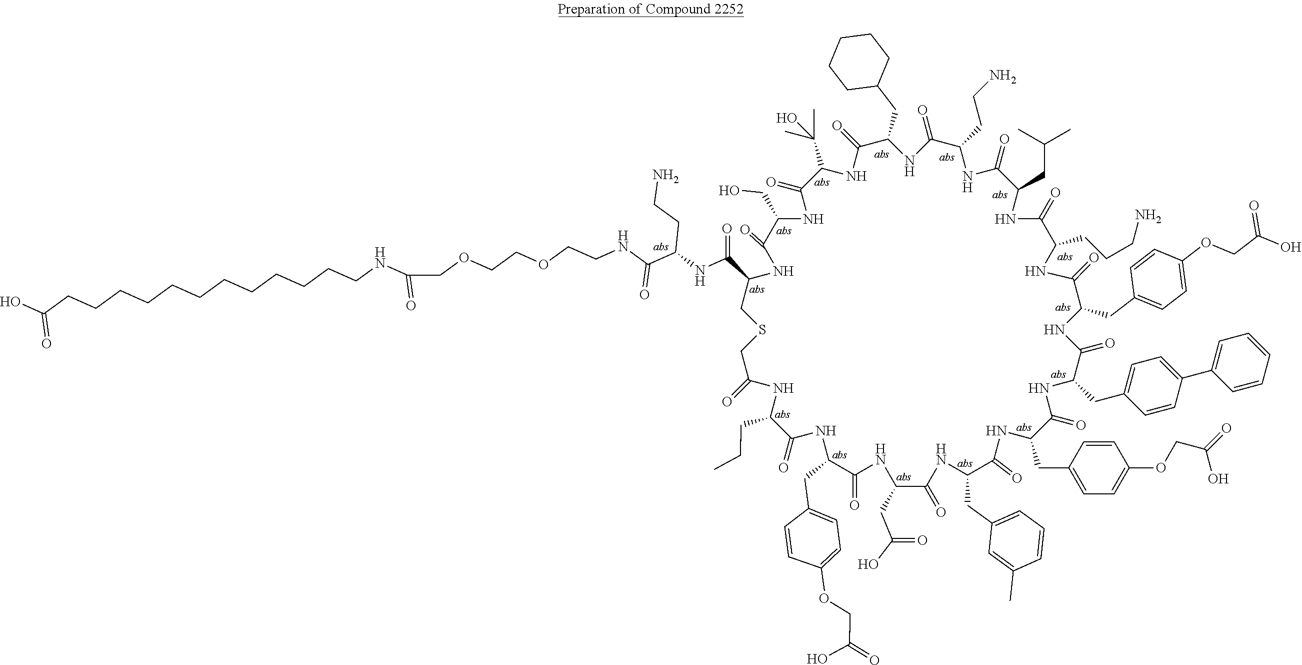

Compound 2252 was prepared, using 2-Chlorotrityl resin pre-loaded with 13-((((9H-fluoren-9-yl) methoxy) carbonyl)amino)tridecanoic acid on a 30 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Nva-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.9 mg, and its estimated purity by LCMS analysis was 93.7%. Analysis condition 1: Retention time=1.91 min; ESI-MS(+) m/z [M+2H]²⁺: 1282.

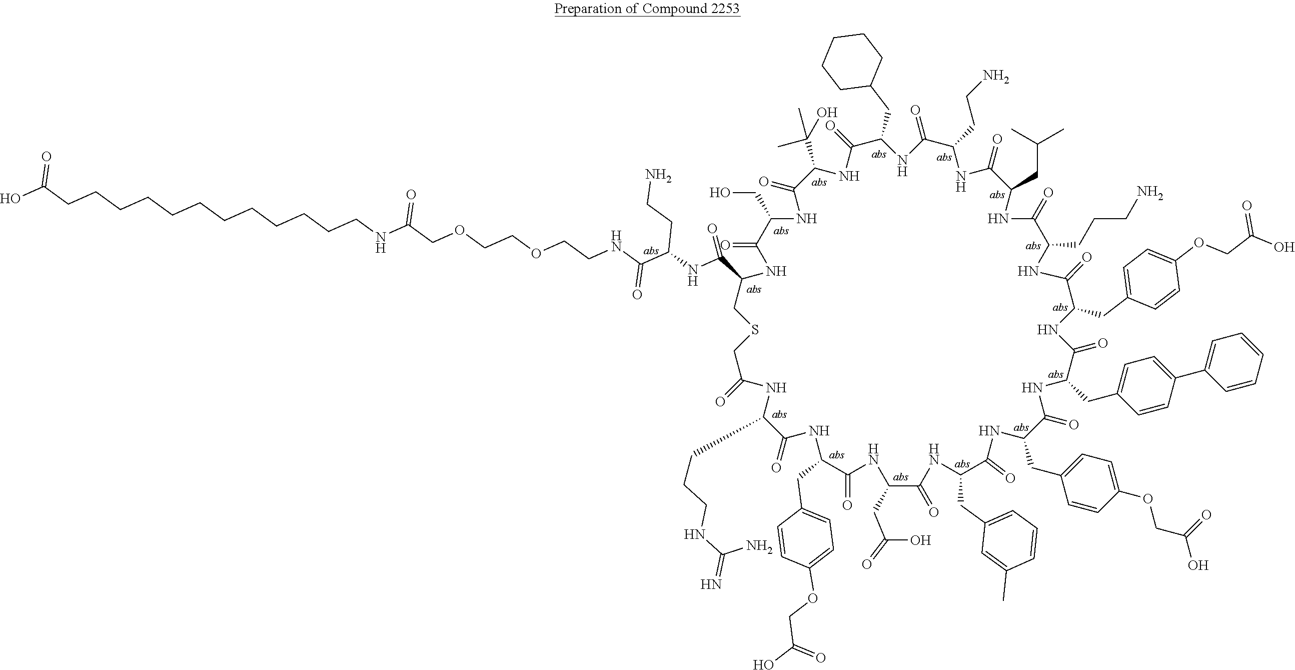 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2253 was prepared, using 2-Chlorotrityl resin pre-loaded with 13-((((9H-fluoren-9-yl) methoxy) carbonyl)amino)tridecanoic acid on a 25 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 22% B, 22-62% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 13 mg, and its estimated purity by LCMS analysis was 89.3%. Analysis condition 2: Retention time=1.62 min; ESI-MS(+) m/z [M+3H]³⁺: 874.2.

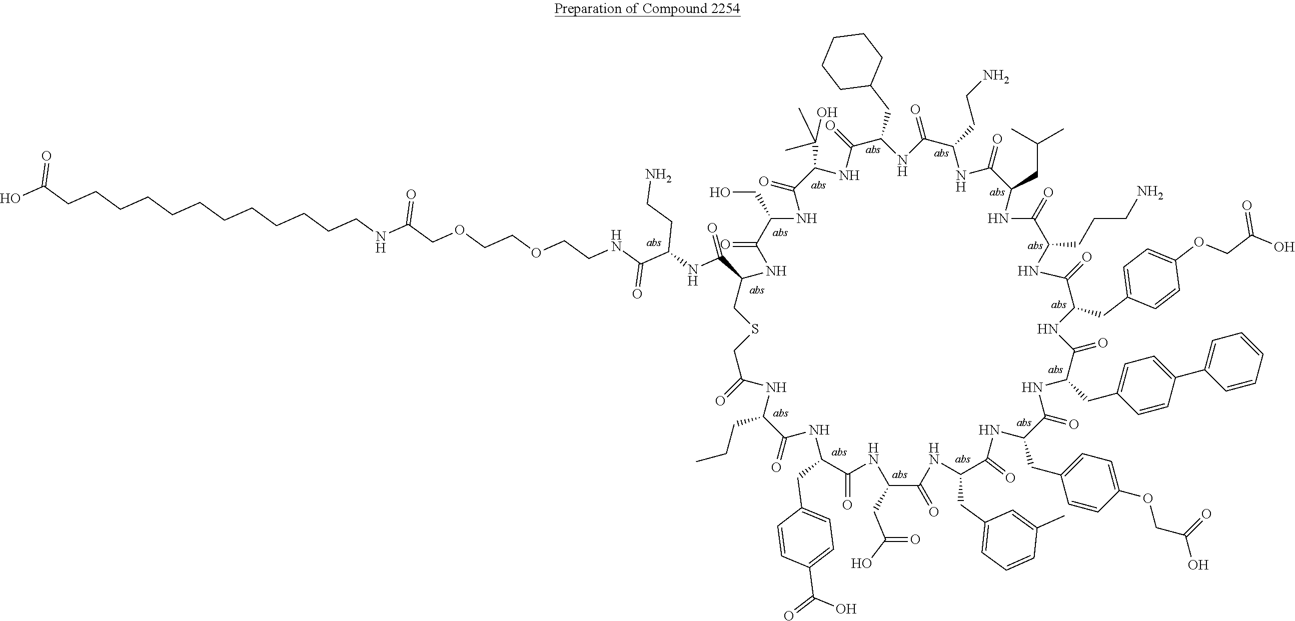 [SMILES: CCC[C@@H]1NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Compound 2254 was prepared, using 2-Chlorotrityl resin pre-loaded with 13-((((9H-fluoren-9-yl) methoxy) carbonyl)amino)tridecanoic acid on a 25 μmol scale, following the general synthetic sequence described for the preparation of Example 1003 composed of the following general procedures: “Prelude Resin-swelling procedure”, “Prelude Single-coupling procedure”, or “Prelude Double-coupling procedure”, “Symphony X Resin-swelling procedure”, “Symphony X Single-coupling procedure”, “Symphony X Single-Coupling Manual Addition Procedure A” was followed with Fmoc-Nva-OH, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 11.1 mg, and its estimated purity by LCMS analysis was 95.5%. Analysis condition 2: Retention time=1.86 min; ESI-MS(+) m/z [M+2H]²⁺: 1267.1.

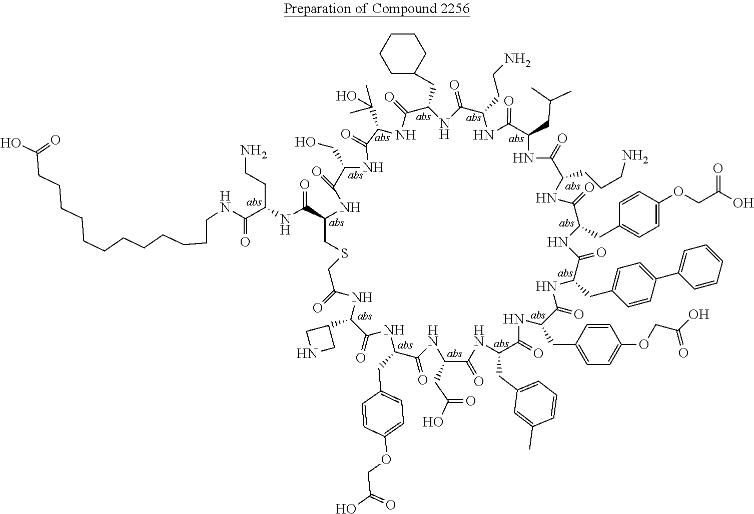 [SMILES: Cc1cccc(C[C@@H]2NC(=O)[C@H](CC(=O)O)NC(=O)[C@H](Cc3ccc(OCC(=O)O)cc3)NC(=O)[C@H](C3CNC3)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2256 was prepared on a 25 μmol scale. The yield of the product was 0.7 mg, and its estimated purity by LCMS analysis was 91.8%. Analysis condition B: Retention time=1.72 min; ESI-MS(+) m/z [M+2H]²⁺: 1216.1.

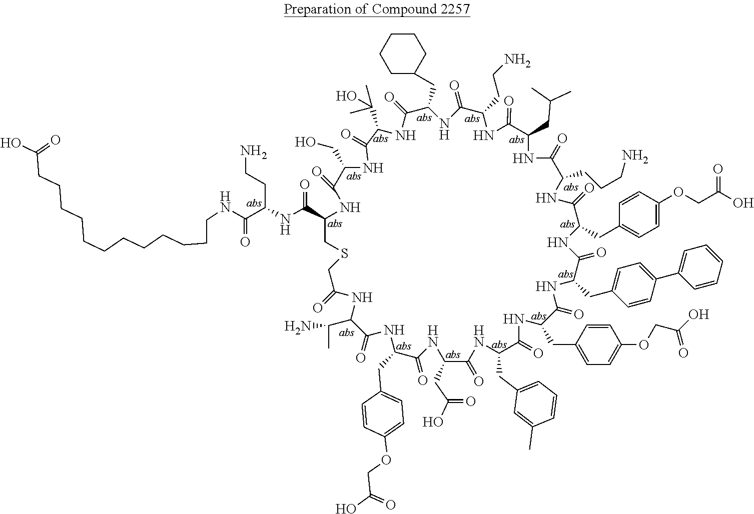 [SMILES: Cc1cccc(C[C@@H]2NC(=O)[C@H](CC(=O)O)NC(=O)[C@H](Cc3ccc(OCC(=O)O)cc3)NC(=O)C([C@H](C)N)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2257 was prepared on a 25 μmol scale. The yield of the product was 6.3 mg, and its estimated purity by LCMS analysis was 92.2%. Analysis condition A: Retention time=1.59 min; ESI-MS(+) m/z [M+2H]²⁺: 1209.9.

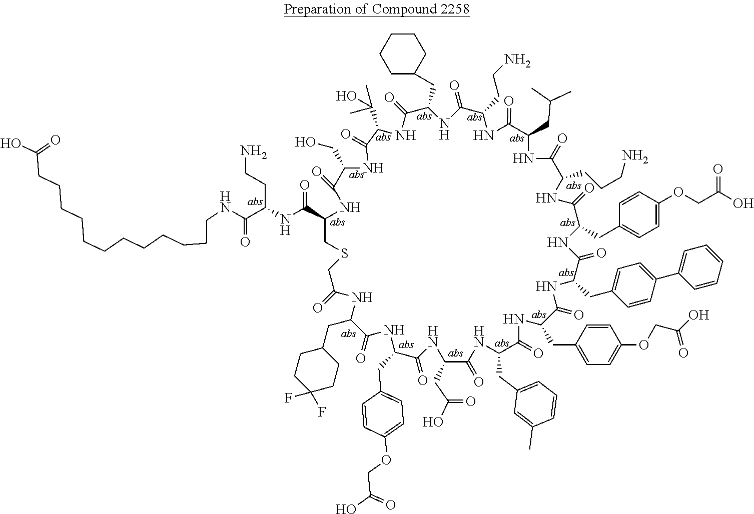 [SMILES: Cc1cccc(C[C@@H]2NC(=O)[C@H](CC(=O)O)NC(=O)[C@H](Cc3ccc(OCC(=O)O)cc3)NC(=O)C(CC3CCC(F)(F)CC3)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2258 was prepared on a 25 μmol scale. The yield of the product was 4.4 mg, and its estimated purity by LCMS analysis was 95.5%. Analysis condition A: Retention time=1.52 min; ESI-MS(+) m/z [M+2H]²⁺: 1254.3.

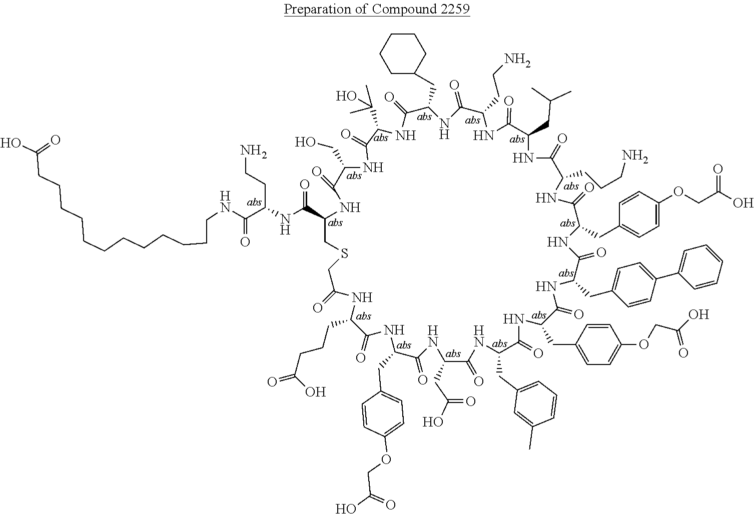 [SMILES: Cc1cccc(C[C@@H]2NC(=O)[C@H](CC(=O)O)NC(=O)[C@H](Cc3ccc(OCC(=O)O)cc3)NC(=O)[C@H](CCCC(=O)O)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2259 was prepared on a 25 μmol scale. The yield of the product was 4.9 mg, and its estimated purity by LCMS analysis was 95.5%. Analysis condition A: Retention time=1.41 min; ESI-MS(+) m/z [M+3H]³⁺: 821.1.

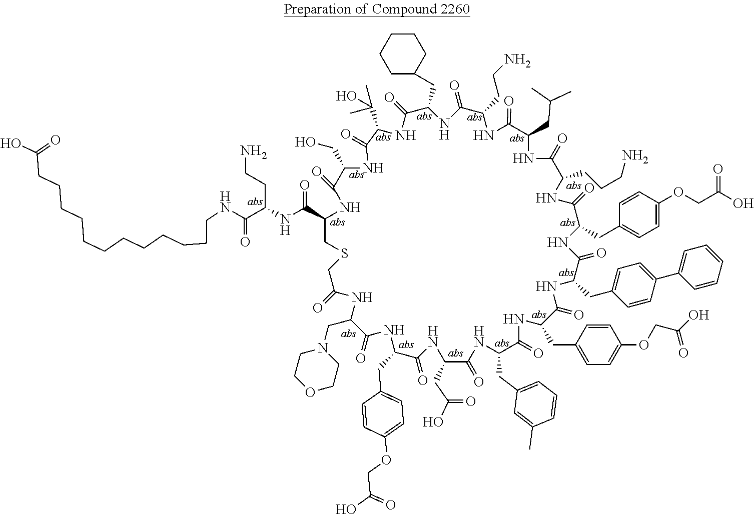 [SMILES: Cc1cccc(C[C@@H]2NC(=O)[C@H](CC(=O)O)NC(=O)[C@H](Cc3ccc(OCC(=O)O)cc3)NC(=O)C(CN3CCOCC3)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2260 was prepared on a 25 μmol scale. The yield of the product was 19.8 mg, and its estimated purity by LCMS analysis was 100%. Analysis condition A: Retention time=1.6 min; ESI-MS(+) m/z [M+2H]²⁺: 1238.1.

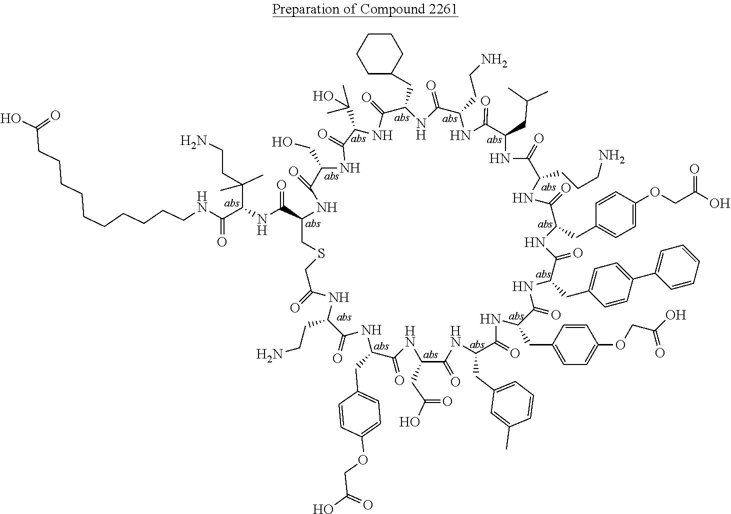 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@H](C(=O)NCCCCCCCCCCC(=O)O)C(C)(C)CCN)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2261 was prepared on a 50 μmol scale. The yield of the product was 2.9 mg, and its estimated purity by LCMS analysis was 96.8%. Analysis condition B: Retention time=1.67 min; ESI-MS(+) m/z [M+2H]²⁺: 1216.9.

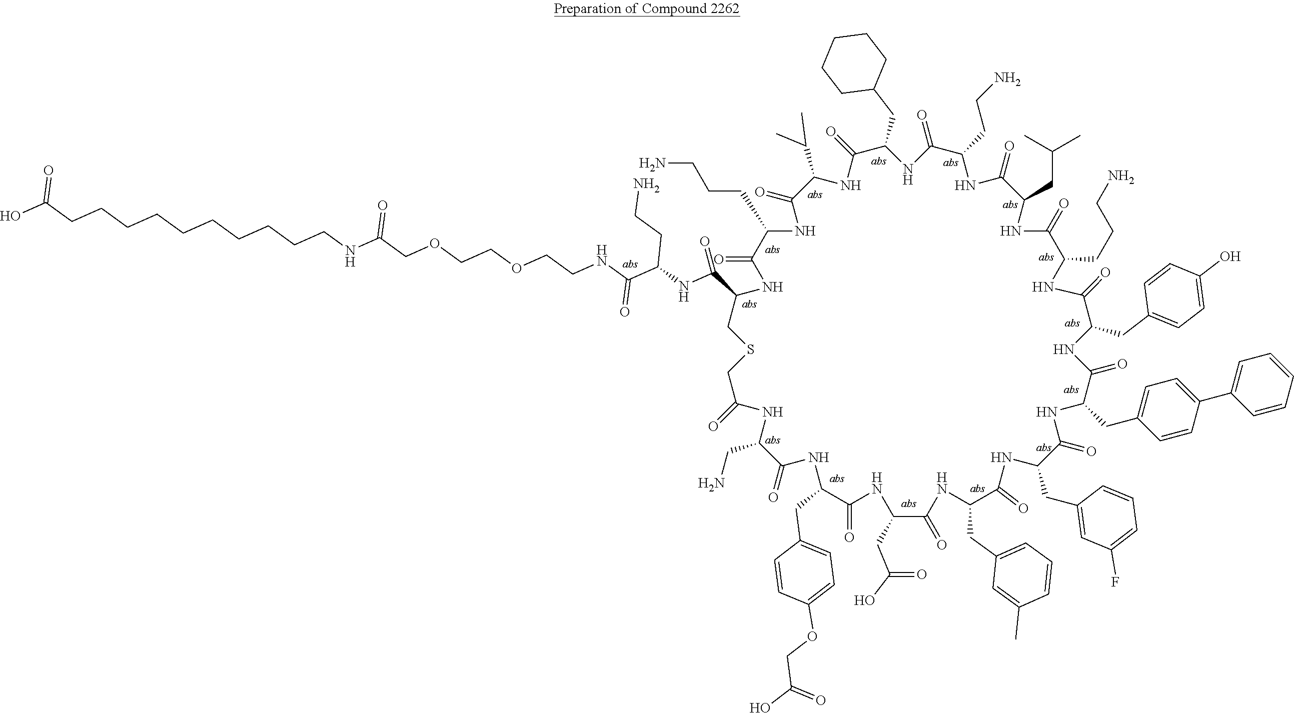 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3cccc(F)c3)NC2=O)c1]

Compound 2262 was prepared on a 50 μmol scale. The yield of the product was 56.7 mg, and its estimated purity by LCMS analysis was 100%. Analysis condition A: Retention time=1.65 min; ESI-MS(+) m/z [M+2H]²⁺: 1210.1.

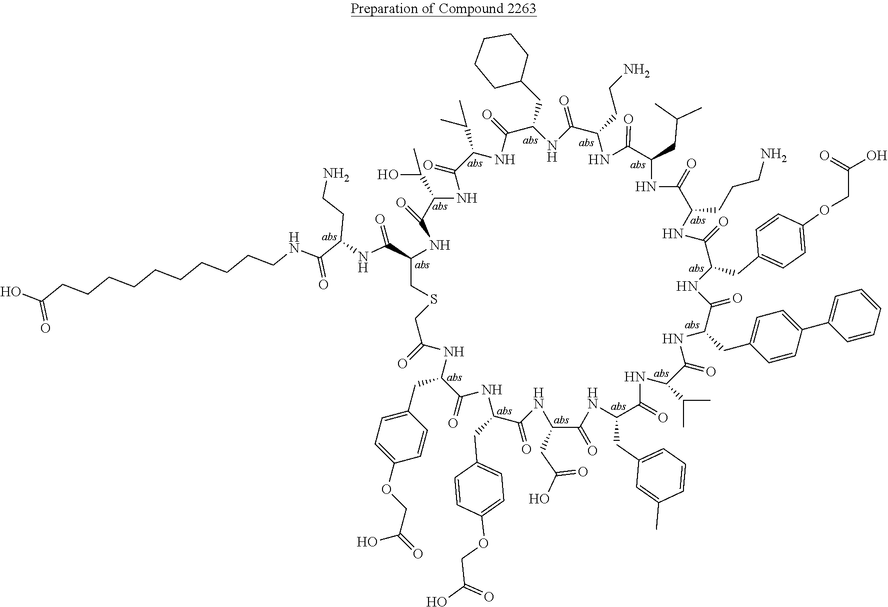

Compound 2263 was prepared on a 50 μmol scale. The yield of the product was 11 mg, and its estimated purity by LCMS analysis was 95.4%. Analysis condition A: Retention time=1.37 min; ESI-MS(+) m/z [M+2H]²⁺: 1194.5.

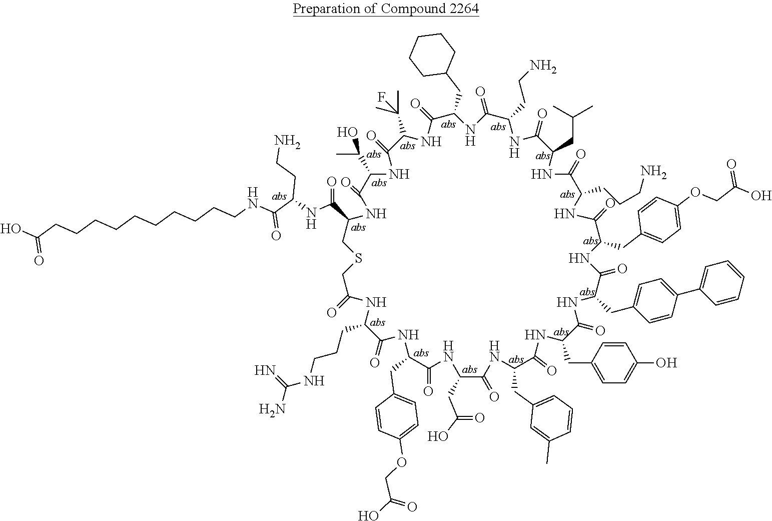

Compound 2264 was prepared on a 50 μmol scale. The yield of the product was 21.7 mg, and its estimated purity by LCMS analysis was 95.1%. Analysis condition B: Retention time=1.63 min; ESI-MS(+) m/z [M+2H]²⁺: 1203.2.

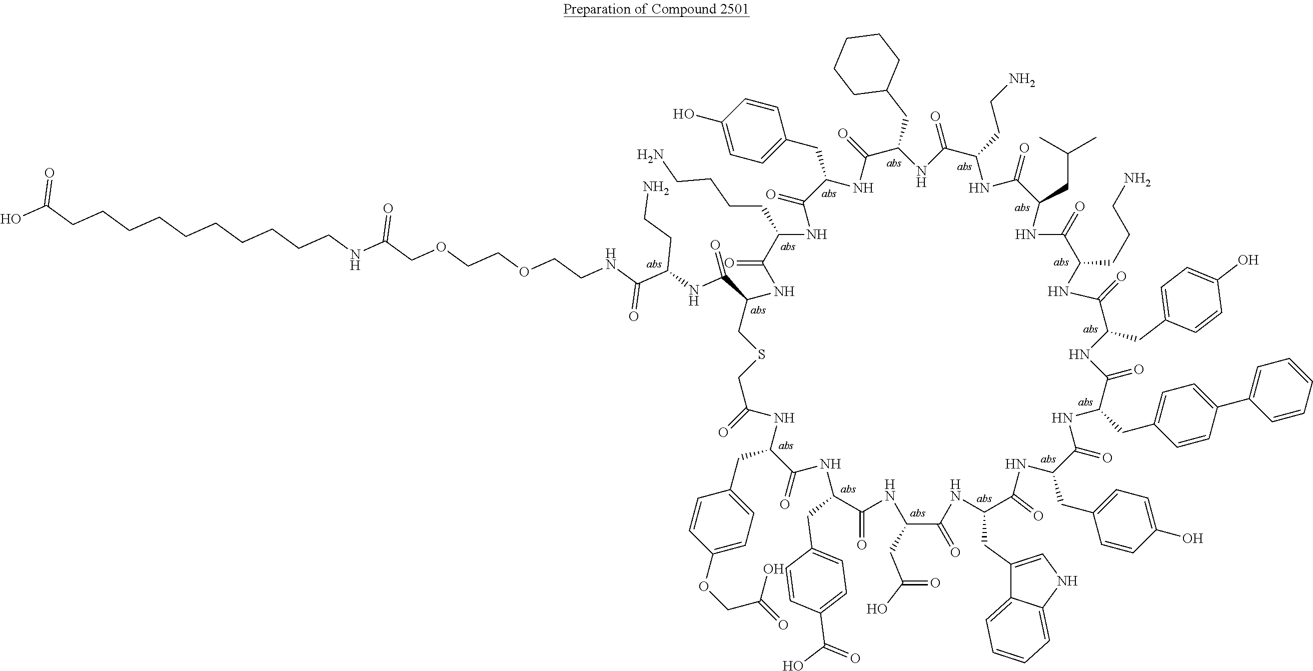 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](Cc2ccc(O)cc2)NC(=O)[C@H](CC2CCCCC2)NC(=O)[C@H](CCN)NC1=O]

Compound 2501 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 34.2 mg, and its estimated purity by LCMS analysis was 100%. Analysis condition A: Retention time=1.55 min; ESI-MS(+) m/z [M+2H]²⁺: 1313.1.

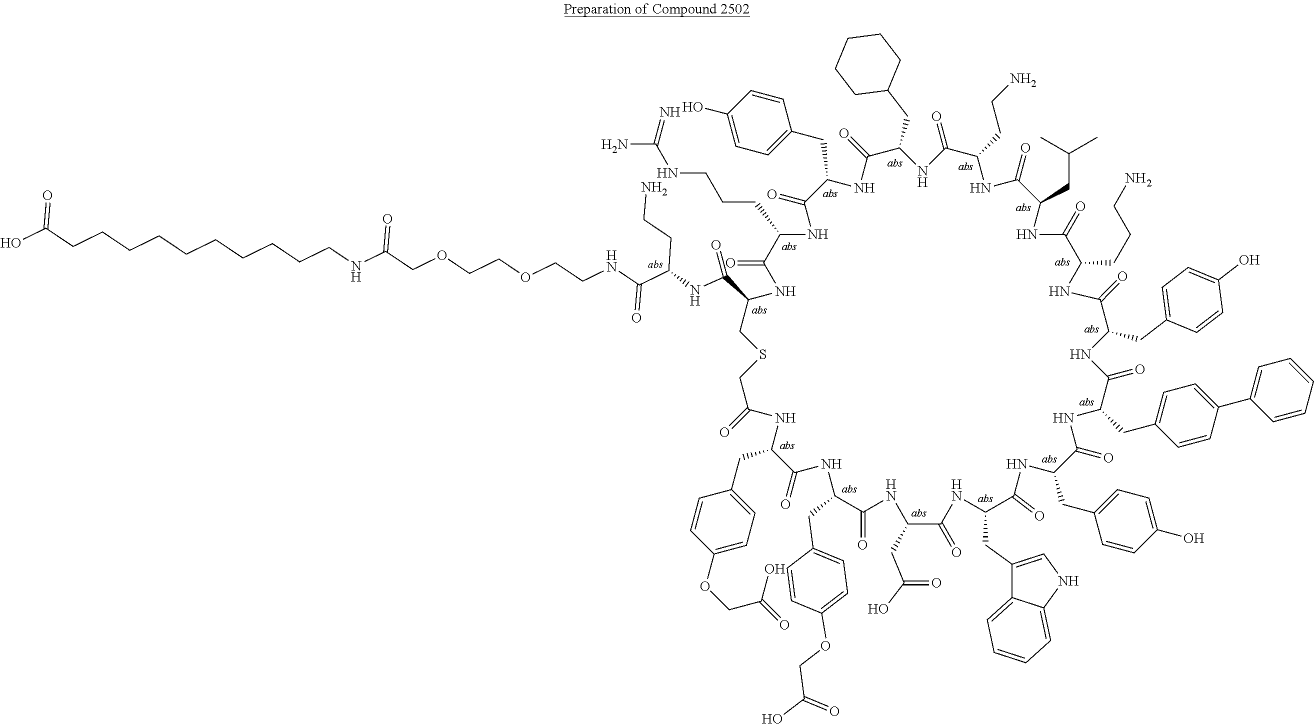 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCNC(=N)N)NC(=O)[C@H](Cc2ccc(O)cc2)NC(=O)[C@H](CC2CCCCC2)NC(=O)[C@H](CCN)NC1=O]

Compound 2502 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 69.3 mg, and its estimated purity by LCMS analysis was 97.8%. Analysis condition A: Retention time=1.46 min; ESI-MS(+) m/z [M+3H]³⁺: 895.2.

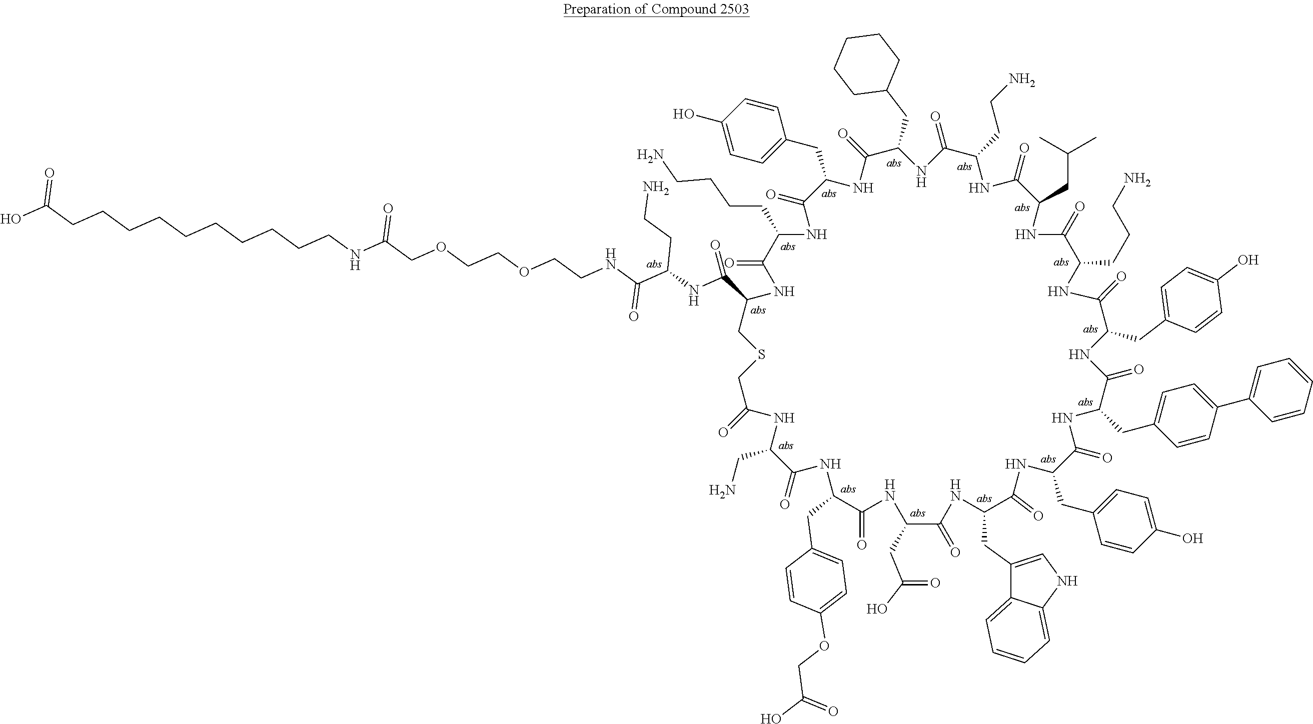 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](Cc2ccc(O)cc2)NC(=O)[C@H](CC2CCCCC2)NC(=O)[C@H](CCN)NC1=O]

Compound 2503 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 19.8 mg, and its estimated purity by LCMS analysis was 98.5%. Analysis condition A: Retention time=1.55 min; ESI-MS(+) m/z [M+3H]³⁺: 841.1.

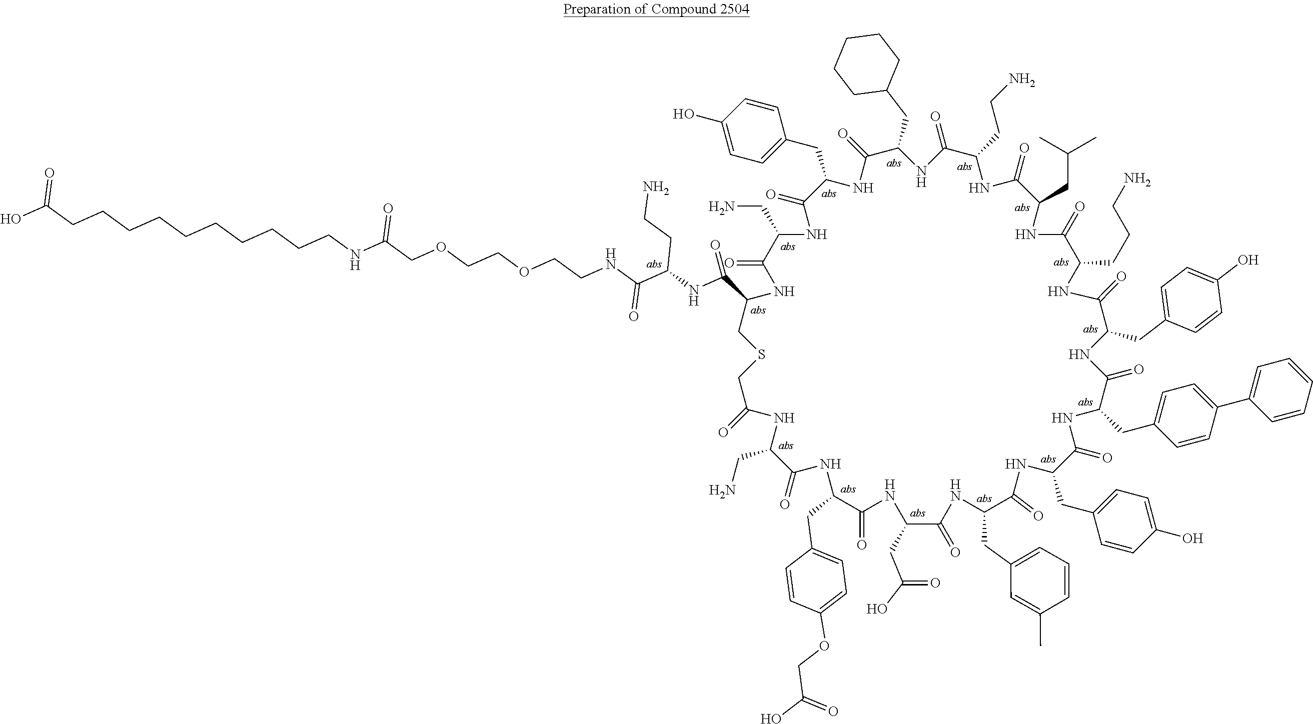 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2504 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 37.4 mg, and its estimated purity by LCMS analysis was 100%. Analysis condition A: Retention time=1.52 min; ESI-MS(+) m/z [M+3H]³⁺: 818.2.

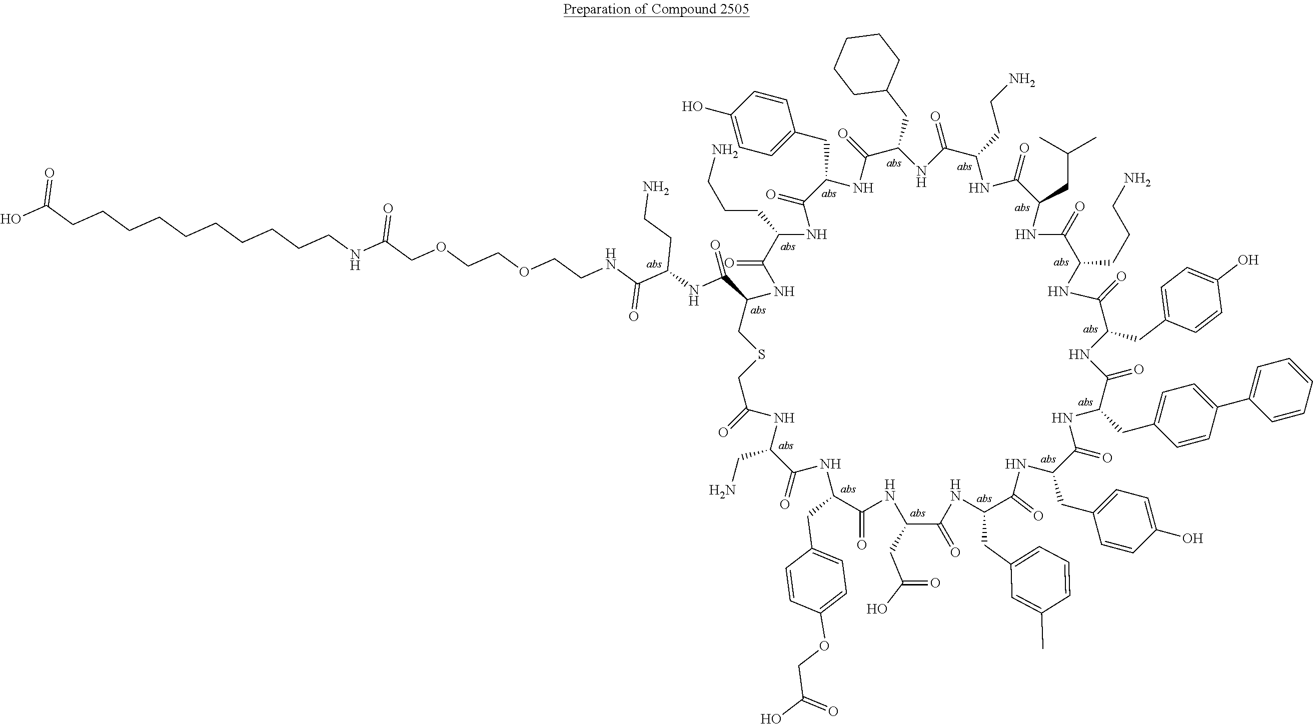 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2505 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 25.3 mg, and its estimated purity by LCMS analysis was 97.4%. Analysis condition B: Retention time=1.56 min; ESI-MS(+) m/z [M+3H]³⁺: 828.

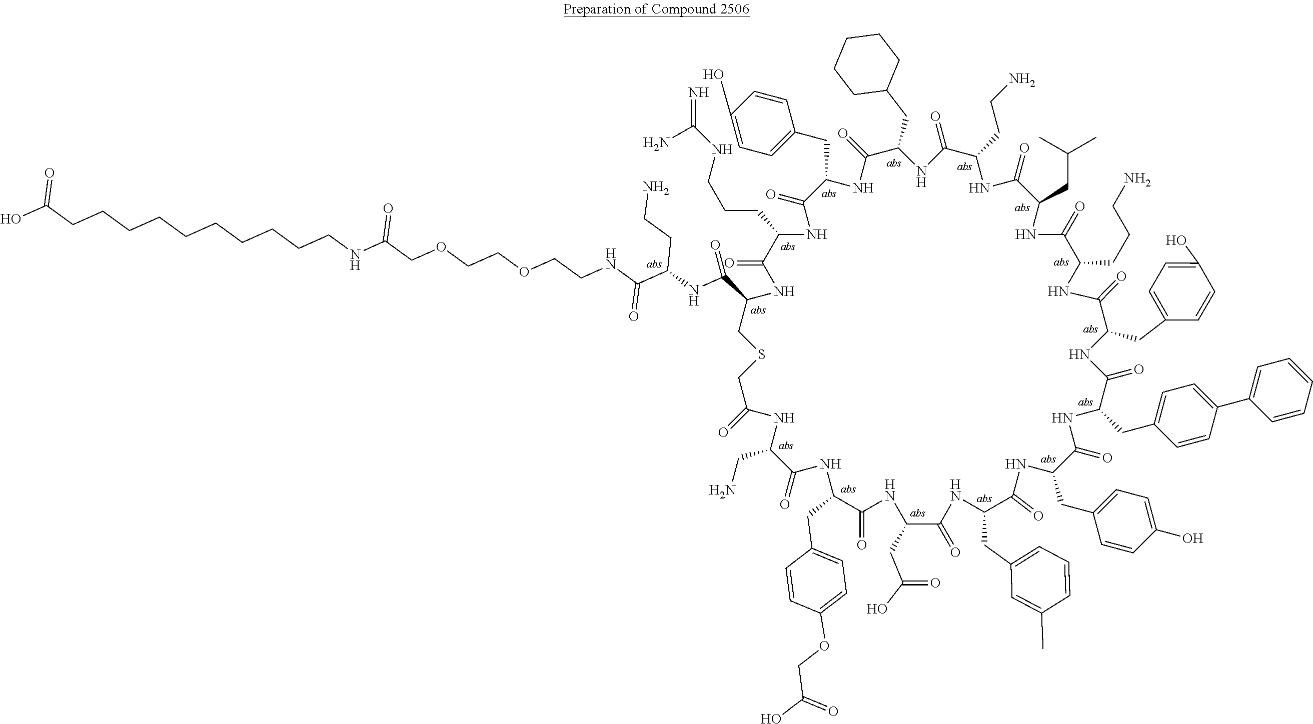 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCNC(=N)N)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2506 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 24.4 mg, and its estimated purity by LCMS analysis was 98.4%. Analysis condition B: Retention time=1.56 min; ESI-MS(+) m/z [M+3H]³⁺: 842.1.

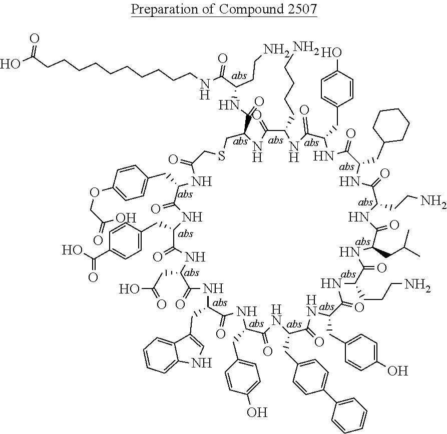 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCCN)NC(=O)[C@H](Cc2ccc(O)cc2)NC(=O)[C@H](CC2CCCCC2)NC(=O)[C@H](CCN)NC1=O]

Compound 2507 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-52% B over 20 minutes, then a 2-minute hold at centrifugal evaporation. The yield of the product was 38.7 mg, and its estimated purity by LCMS analysis was 96.1%. Analysis condition A: Retention time=1.43 min; ESI-MS(+) m/z [M+3H]³⁺: 827.5.

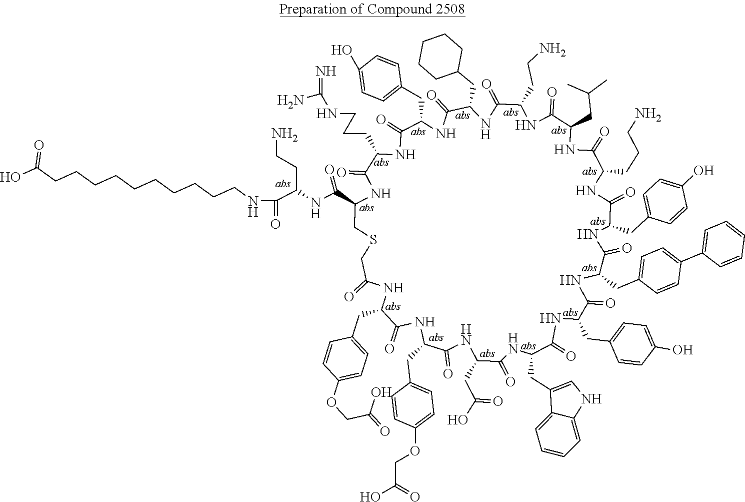

Compound 2508 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 7.1 mg, and its estimated purity by LCMS analysis was 96.7%. Analysis condition B: Retention time=1.5 min; ESI-MS(+) m/z [M+3H]³⁺: 846.4.

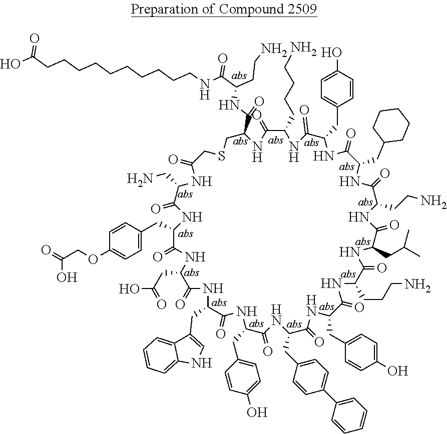 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](Cc2ccc(O)cc2)NC(=O)[C@H](CC2CCCCC2)NC(=O)[C@H](CCN)NC1=O]

Compound 2509 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20.7 mg, and its estimated purity by LCMS analysis was 98.3%. Analysis condition A: Retention time=1.55 min; ESI-MS(+) m/z [M+2H]²⁺: 1188.2.

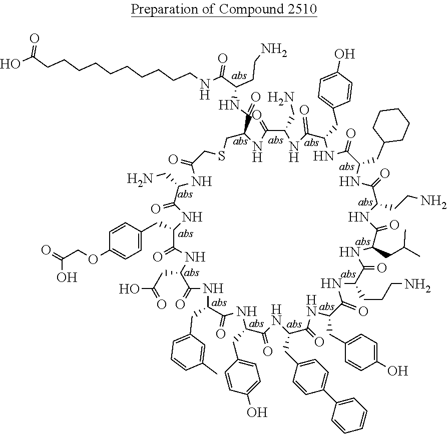 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2510 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 28.2 mg, and its estimated purity by LCMS analysis was 95.4%. Analysis condition A: Retention time=1.54 min; ESI-MS(+) m/z [M+3H]³⁺: 770.3.

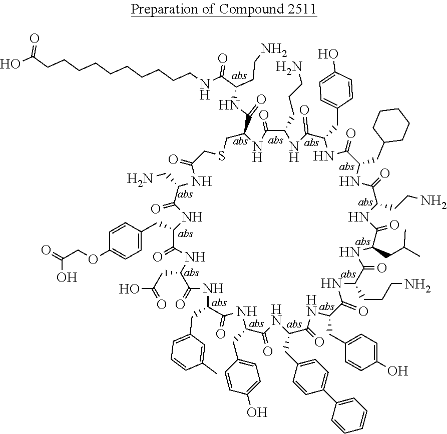 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2511 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at by MS and UV signals. Fractions containing the desired product were combined and dried via centrifugal evaporation. The yield of the product was 26.1 mg, and its estimated purity by LCMS analysis was 97.7%. Analysis condition B: Retention time=1.44 min; ESI-MS(+) m/z [M+3H]³⁺: 779.3.

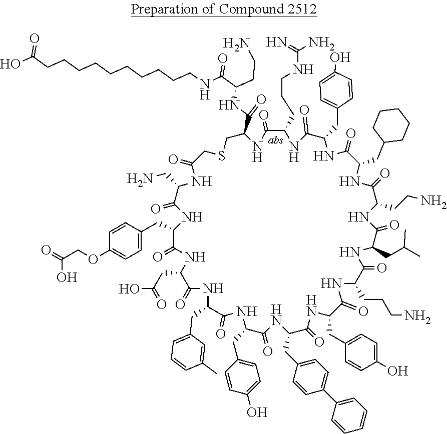 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCNC(=N)N)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2512 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 48.9 mg, and its estimated purity by LCMS analysis was 99.4%. Analysis condition A: Retention time=1.58 min; ESI-MS(+) m/z [M+3H]³⁺: 793.2.

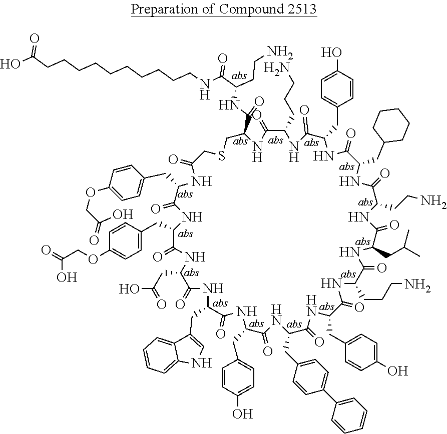 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCN)NC(=O)[C@H](Cc2ccc(O)cc2)NC(=O)[C@H](CC2CCCCC2)NC(=O)[C@H](CCN)NC1=O]

Compound 2513 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.9 mg, and its estimated purity by LCMS analysis was 94.2%. Analysis condition B: Retention time=1.53 min; ESI-MS(+) m/z [M+2H]²⁺: 1247.7.

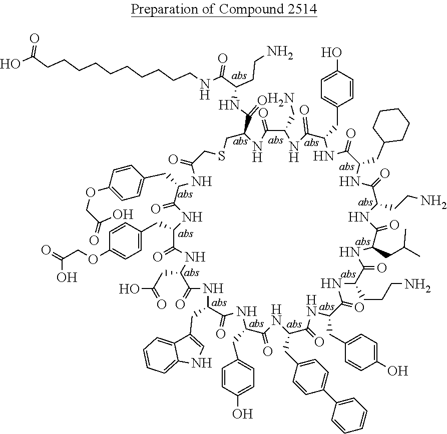 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CN)NC(=O)[C@H](Cc2ccc(O)cc2)NC(=O)[C@H](CC2CCCCC2)NC(=O)[C@H](CCN)NC1=O]

Compound 2514 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 24% B, 24-46% B over 25 minutes, then a 2-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34 mg, and its estimated purity by LCMS analysis was 94.2%. Analysis condition B: Retention time=1.57 min; ESI-MS(+) m/z [M+2H]²⁺: 1234.2.

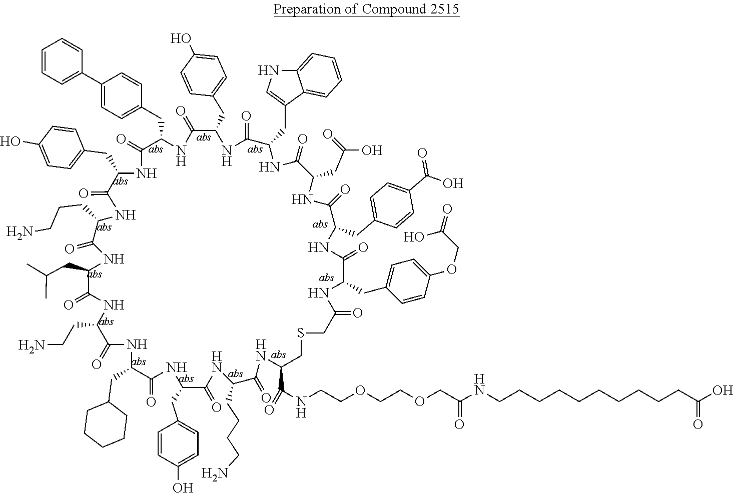 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCCN)NC(=O)[C@H](Cc2ccc(O)cc2)NC(=O)[C@H](CC2CCCCC2)NC(=O)[C@H](CCN)NC1=O]

Compound 2515 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 28.7 mg, and its estimated purity by LCMS analysis was 93.7%. Analysis condition B: Retention time=1.61 min; ESI-MS(+) m/z [M+2H]²⁺: 1263.

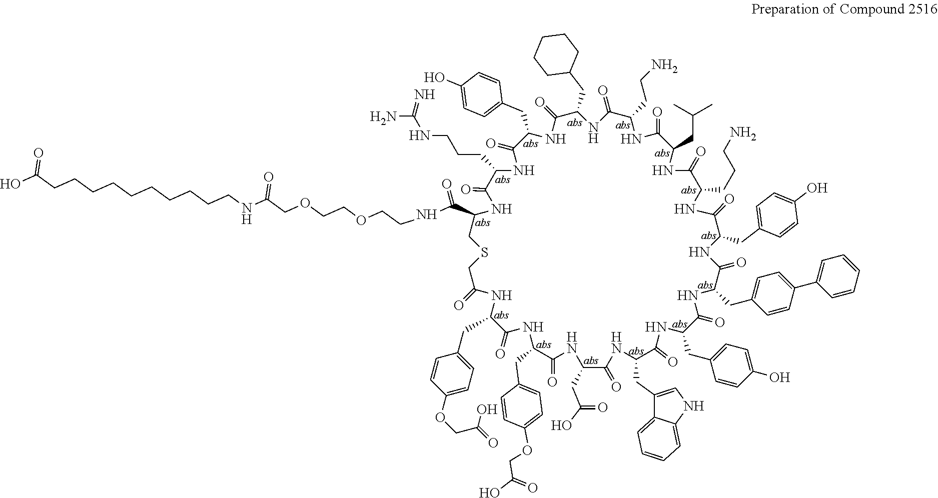 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(=N)N)NC(=O)[C@H](Cc2ccc(O)cc2)NC(=O)[C@H](CC2CCCCC2)NC(=O)[C@H](CCN)NC1=O]

Compound 2516 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 47.5 mg, and its estimated purity by LCMS analysis was 98.8%. Analysis condition A: Retention time=1.55 min; ESI-MS(+) m/z [M+2H]²⁺: 1292.2.

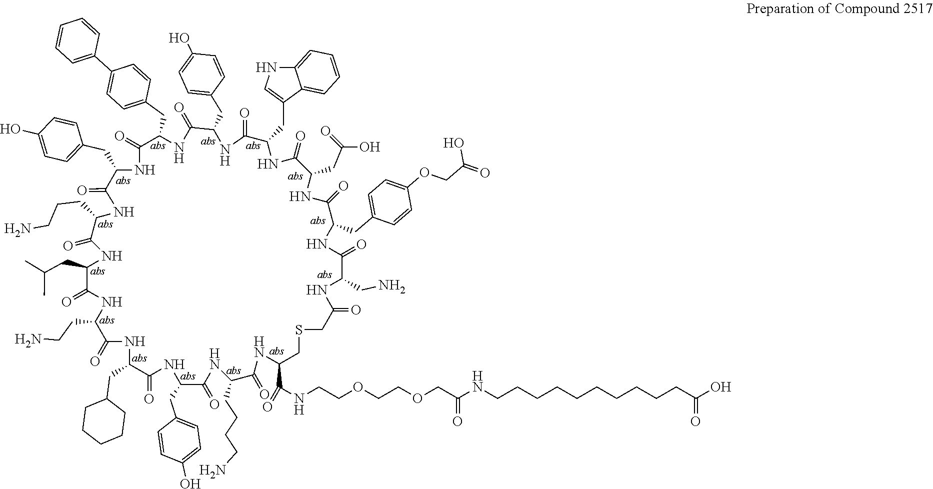 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)NCCCCCCCCCCC(=O)O)NC(=O)[C@H](CCCCN)NC(=O)[C@H](Cc2ccc(O)cc2)NC(=O)[C@H](CC2CCCCC2)NC(=O)[C@H](CCN)NC1=O]

Compound 2517 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 28.3 mg, and its estimated purity by LCMS analysis was 93.9%. Analysis condition B: Retention time=1.5 min; ESI-MS(+) m/z [M+3H]³⁺: 807.1.

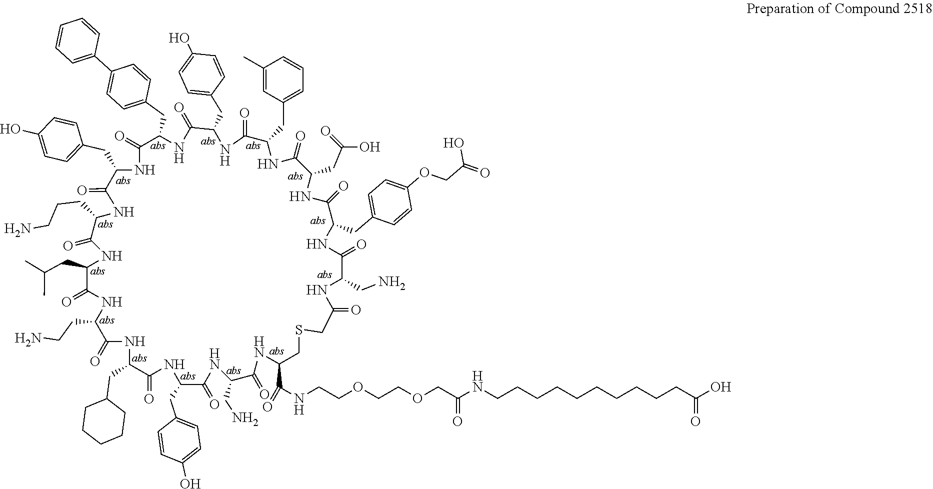 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2518 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 19.4 mg, and its estimated purity by LCMS analysis was 100%. Analysis condition A: Retention time=1.65 min; ESI-MS(+) m/z [M+2H]²⁺: 1177.1.

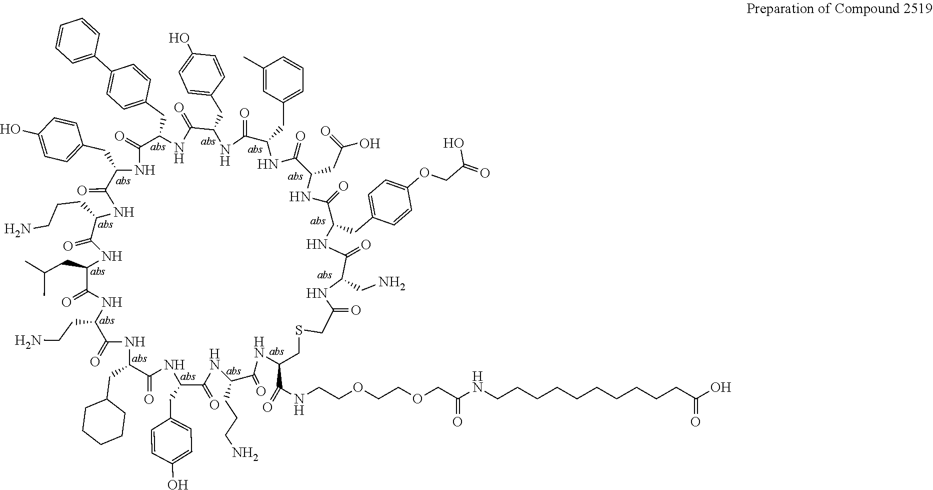 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2519 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 30.5 mg, and its estimated purity by LCMS analysis was 92.5%. Analysis condition A: Retention time=1.55 min; ESI-MS(+) m/z [M+2H]²⁺: 1190.9.

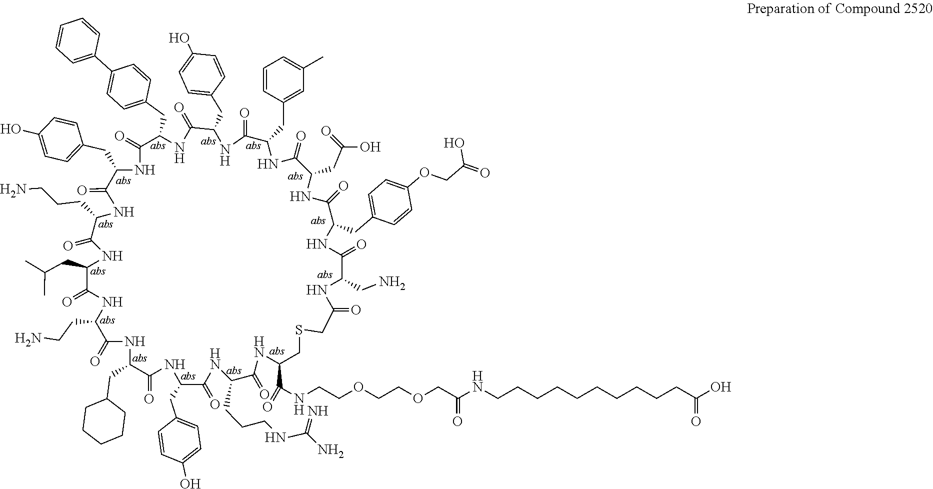 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCNC(=N)N)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2520 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 52.1 mg, and its estimated purity by LCMS analysis was 100%. Analysis condition B: Retention time=1.55 min; ESI-MS(+) m/z [M+2H]²⁺: 1212.1.

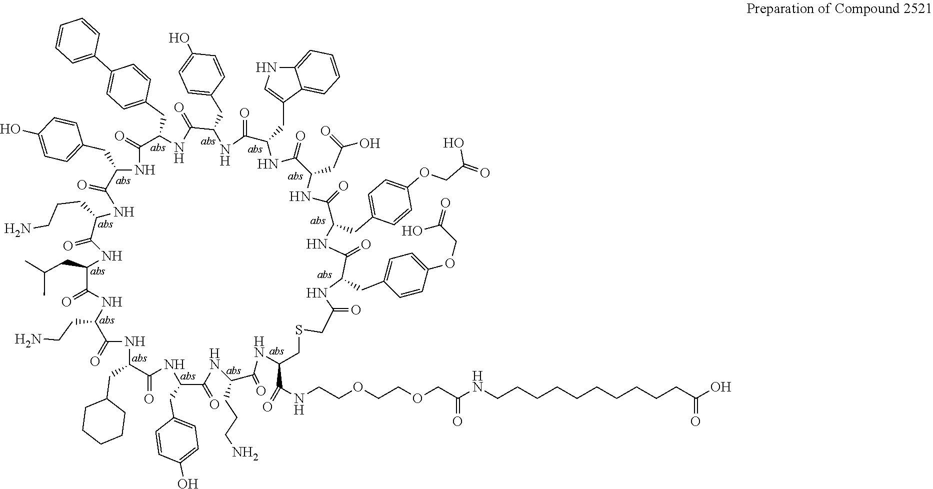 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCN)NC(=O)[C@H](Cc2ccc(O)cc2)NC(=O)[C@H](CC2CCCCC2)NC(=O)[C@H](CCN)NC1=O]

Compound 2521 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 66.1 mg, and its estimated purity by LCMS analysis was 94.1%. Analysis condition B: Retention time=1.64 min; ESI-MS(+) m/z [M+2H]²⁺: 1270.9.

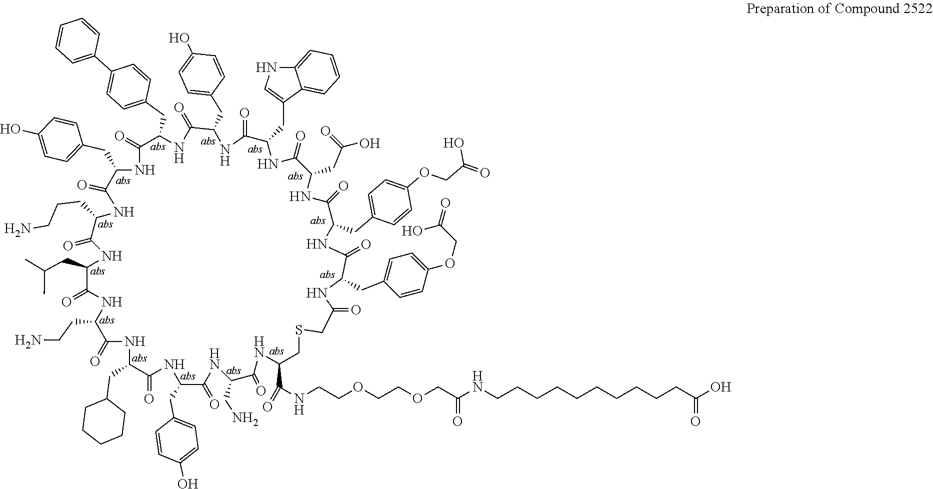 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CN)NC(=O)[C@H](Cc2ccc(O)cc2)NC(=O)[C@H](CC2CCCCC2)NC(=O)[C@H](CCN)NC1=O]

Compound 2522 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 72 mg, and its estimated purity by LCMS analysis was 93.4%. Analysis condition B: Retention time=1.67 min; ESI-MS(+) m/z [M+2H]²⁺: 1257.

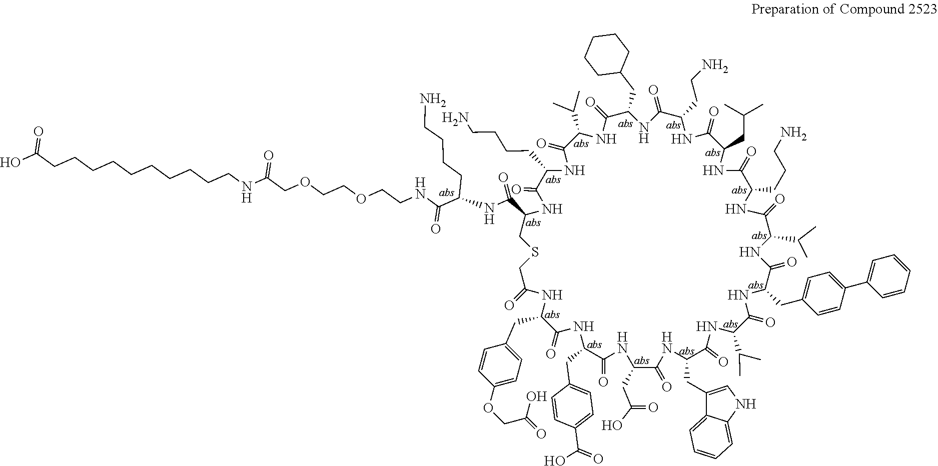 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2523 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 9.8 mg, and its estimated purity by LCMS analysis was 100%. Analysis condition A: Retention time=1.6 min; ESI-MS(+) m/z [M+3H]³⁺: 821.1.

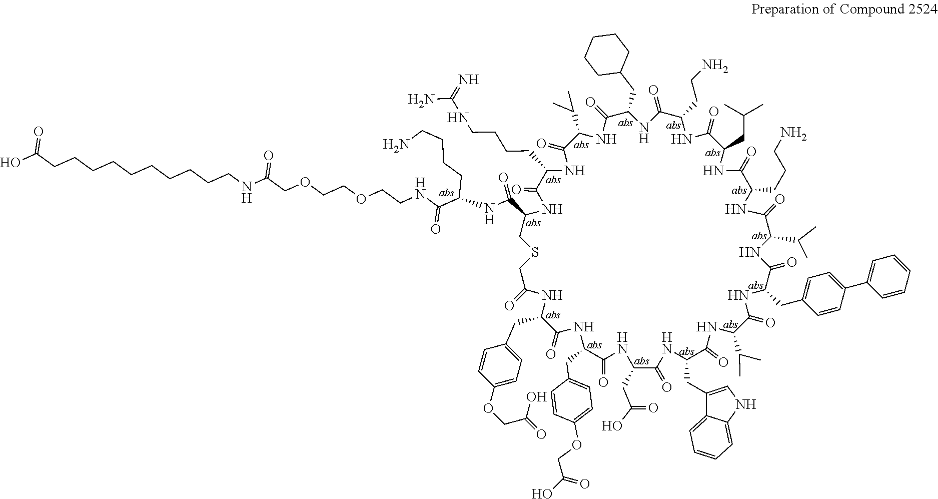 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CCCCNC(=N)N)NC(=O)[C@H](C(C)C)NC(=O)[C@H](CC2CCCCC2)NC(=O)[C@H](CCN)NC1=O]

Compound 2524 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.7 mg, and its estimated purity by LCMS analysis was 100%. Analysis condition A: Retention time=1.63 min; ESI-MS(+) m/z [M+2H]²⁺: 1259.9.

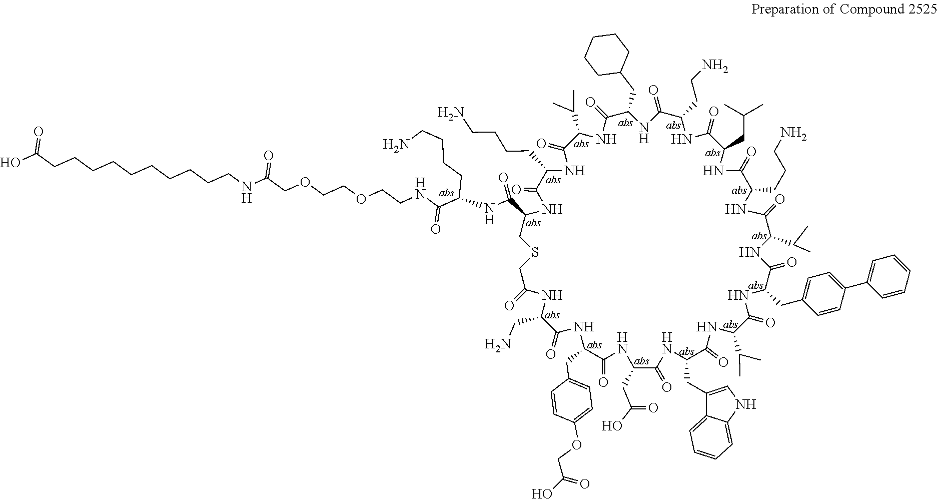 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2525 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-50% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.6 mg, and its estimated purity by LCMS analysis was 94%. Analysis condition A: Retention time=1.56 min; ESI-MS(+) m/z [M+3H]³⁺: 786.2.

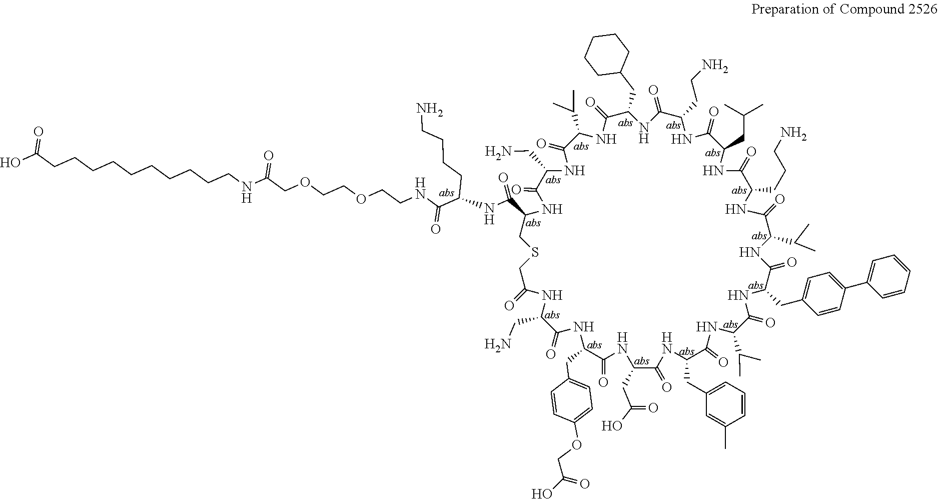 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2526 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 38.6 mg, and its estimated purity by LCMS analysis was 95.9%. Analysis condition B: Retention time=1.55 min; ESI-MS(+) m/z [M+2H]²⁺: 1145.2.

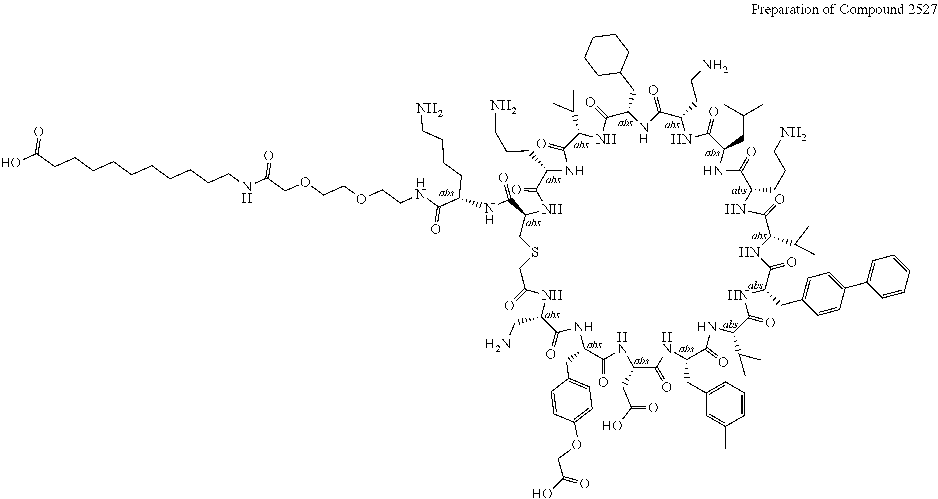 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2527 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 9% B, 9-49% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 35.4 mg, and its estimated purity by LCMS analysis was 100%. Analysis condition A: Retention time=1.6 min; ESI-MS(+) m/z [M+2H]²⁺: 1159.1.

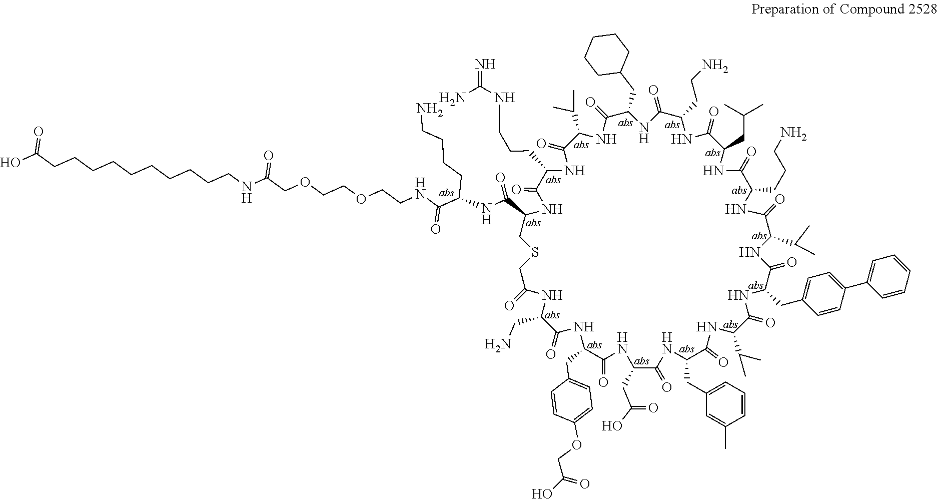 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2528 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 9% B, 9-49% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.6 mg, and its estimated purity by LCMS analysis was 100%. Analysis condition A: Retention time=1.58 min; ESI-MS(+) m/z [M+2H]²⁺: 1180.1.

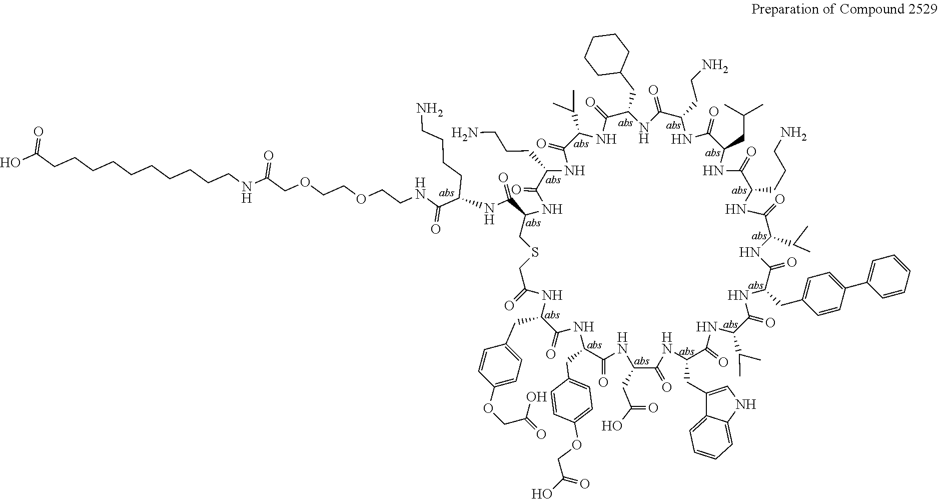 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2529 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 27 mg, and its estimated purity by LCMS analysis was 96.9%. Analysis condition A: Retention time=1.66 min; ESI-MS(+) m/z [M+2H]²⁺: 1239.2.

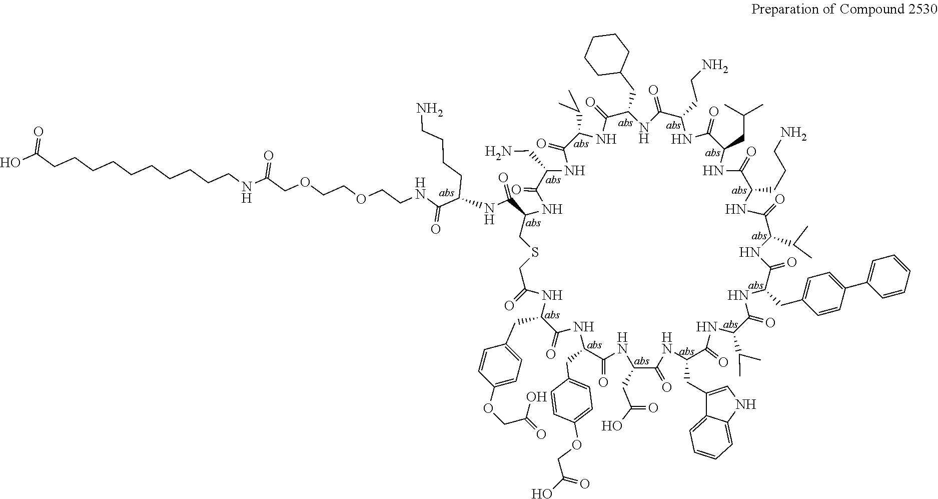 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCCN)C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2530 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 33.6 mg, and its estimated purity by LCMS analysis was 94.3%. Analysis condition B: Retention time=1.65 min; ESI-MS(+) m/z [M+2H]²⁺: 1225.1.

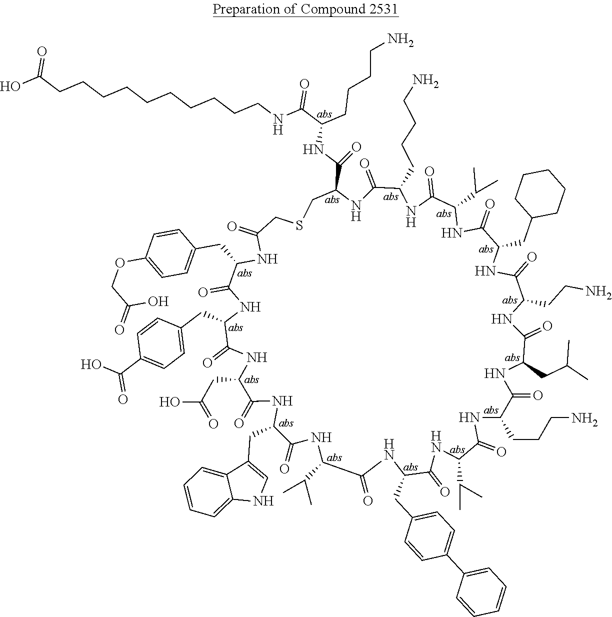 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2531 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5 mg, and its estimated purity by LCMS analysis was 96.1%. Analysis condition B: Retention time=1.55 min; ESI-MS(+) m/z [M+2H]²⁺: 1159.1.

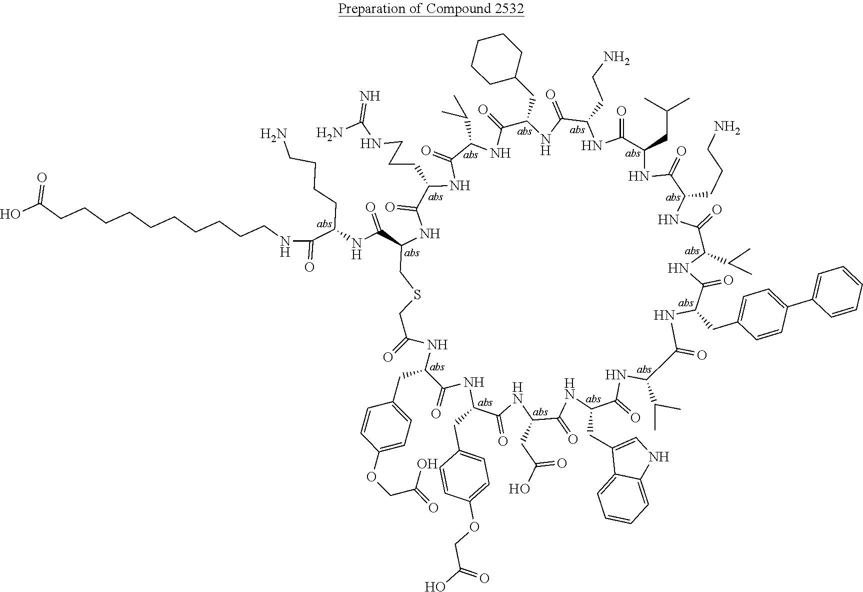 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2532 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.5 mg, and its estimated purity by LCMS analysis was 96.6%. Analysis condition B: Retention time=1.55 min; ESI-MS(+) m/z [M+2H]²⁺: 1187.2.

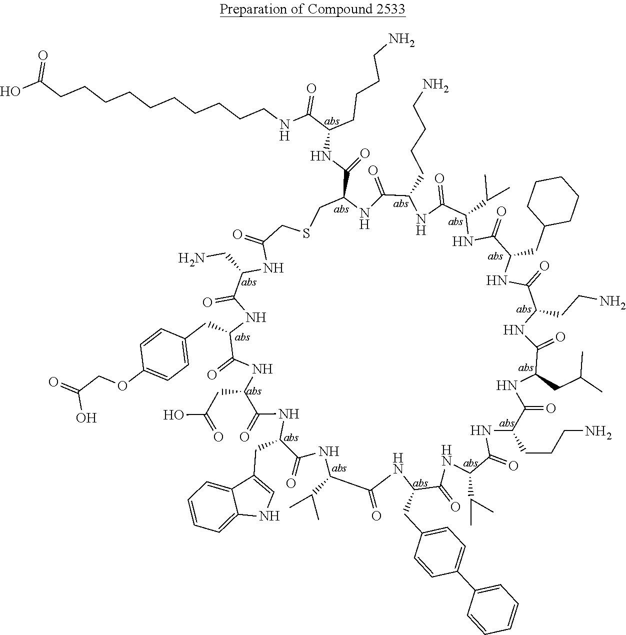 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2533 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.3 mg, and its estimated purity by LCMS analysis was 90.1%. Analysis condition B: Retention time=1.41 min; ESI-MS(+) m/z [M+2H]²⁺: 1106.2.

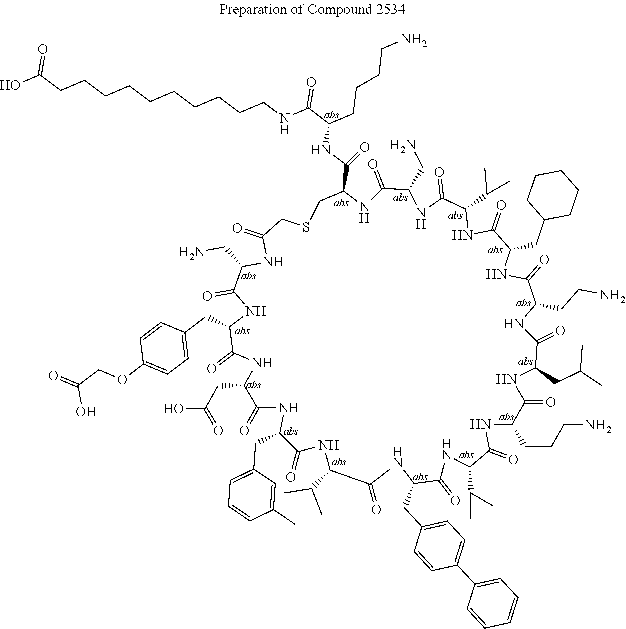 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2534 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-50% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 40.9 mg, and its estimated purity by LCMS analysis was 98.6%. Analysis condition B: Retention time=1.51 min; ESI-MS(+) m/z [M+3H]³⁺: 715.

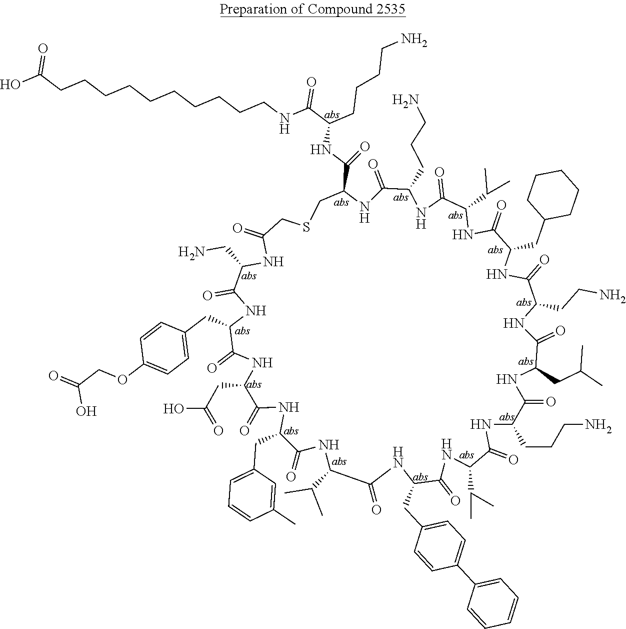 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2535 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 11.6 mg, and its estimated purity by LCMS analysis was 100%. Analysis condition A: Retention time=1.62 min; ESI-MS(+) m/z [M+2H]²⁺: 1087.

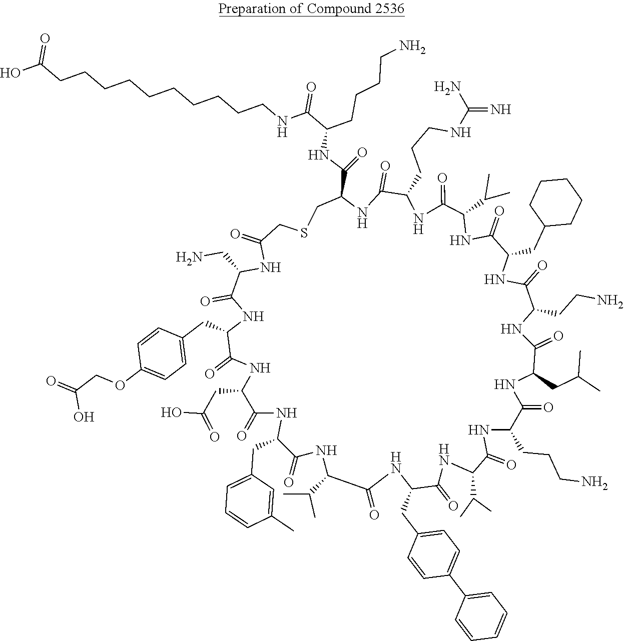 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2536 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 9% B, 9-49% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.2 mg, and its estimated purity by LCMS analysis was 99.3%. Analysis condition B: Retention time=1.38 min; ESI-MS(+) m/z [M+3H]³⁺: 738.7.

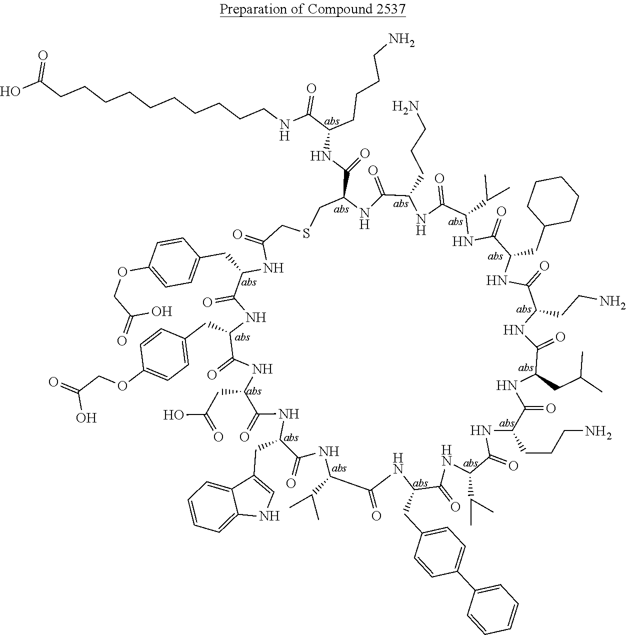 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2537 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.7 mg, and its estimated purity by LCMS analysis was 94.5%. Analysis condition B: Retention time=1.56 min; ESI-MS(+) m/z [M+2H]²⁺: 1166.1.

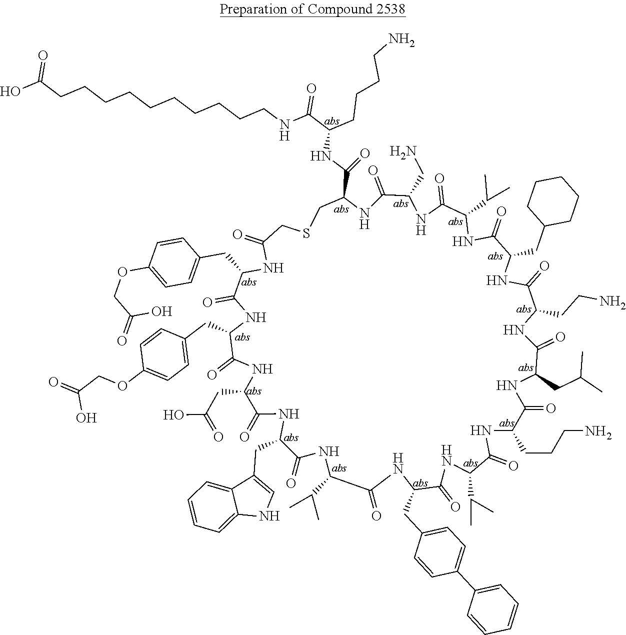 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2538 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 16.4 mg, and its estimated purity by LCMS analysis was 98.8%. Analysis condition B: Retention time=1.57 min; ESI-MS(+) m/z [M+2H]²⁺: 1152.2.

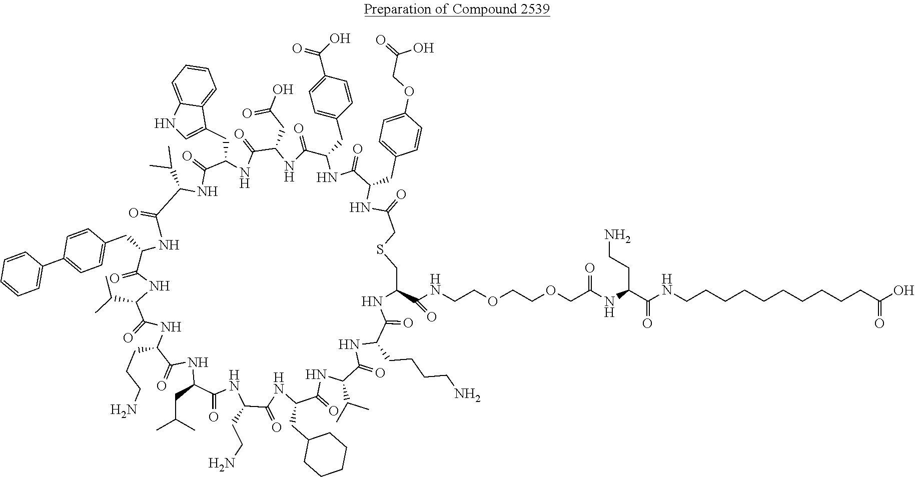 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2539 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8 mg, and its estimated purity by LCMS analysis was 97.2%. Analysis condition B: Retention time=1.55 min; ESI-MS(+) m/z [M+2H]²⁺: 1217.1.

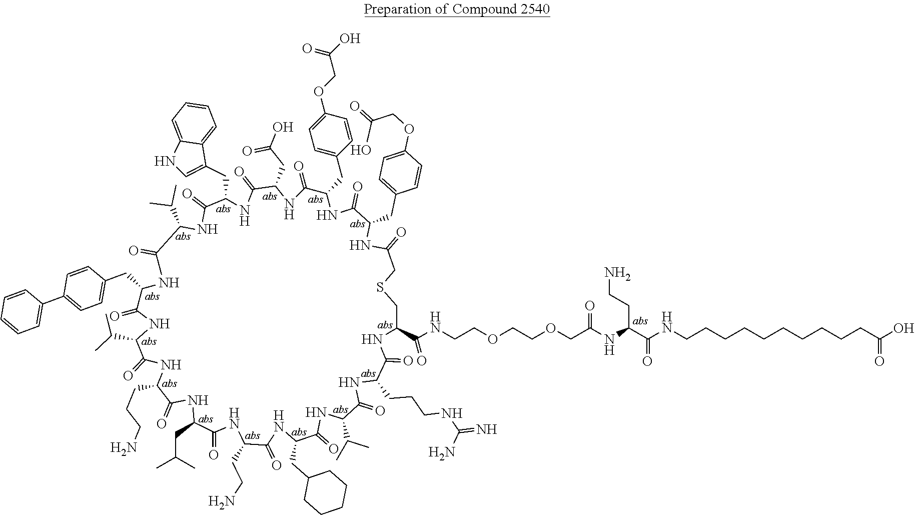 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2540 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.9 mg, and its estimated purity by LCMS analysis was 93.9%. Analysis condition A: Retention time=1.61 min; ESI-MS(+) m/z [M+2H]²⁺: 1246.3.

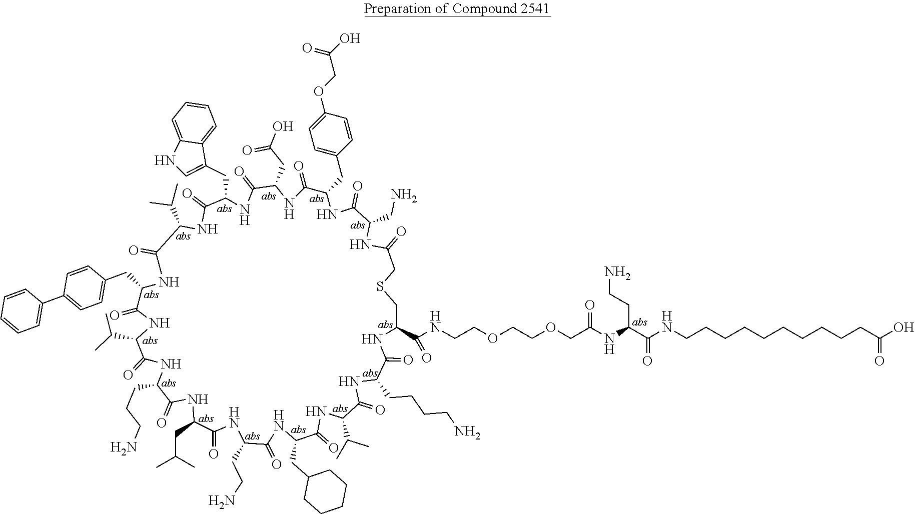 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2541 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.9 mg, and its estimated purity by LCMS analysis was 91.8%. Analysis condition B: Retention time=1.43 min; ESI-MS(+) m/z [M+3H]³⁺: 776.8.

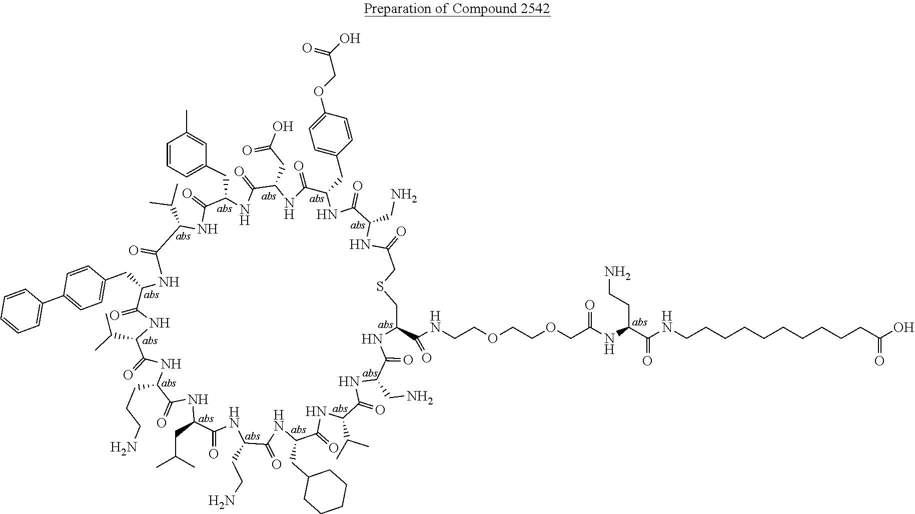 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2542 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 24.3 mg, and its estimated purity by LCMS analysis was 100%. Analysis condition A: Retention time=1.62 min; ESI-MS(+) m/z [M+3H]³⁺: 754.2.

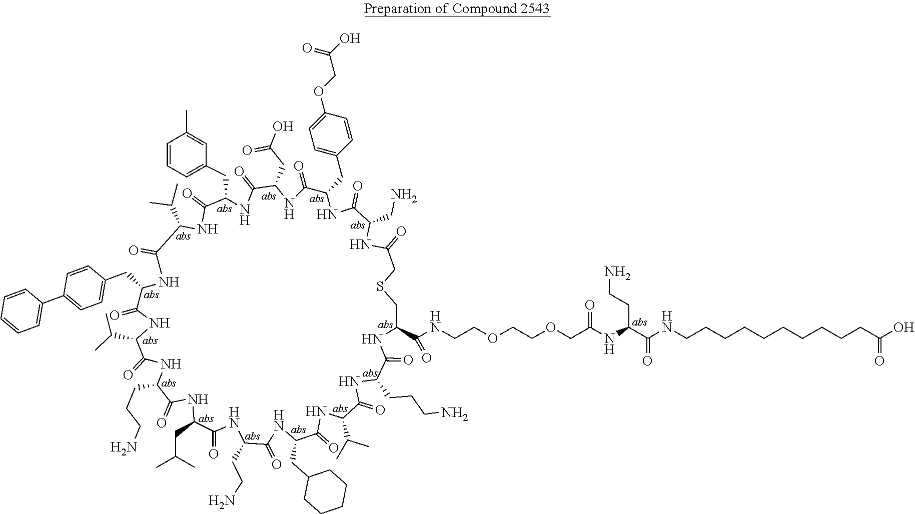 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2543 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 24.5 mg, and its estimated purity by LCMS analysis was 94.2%. Analysis condition B: Retention time=1.48 min; ESI-MS(+) m/z [M+2H]²⁺: 1145.2.

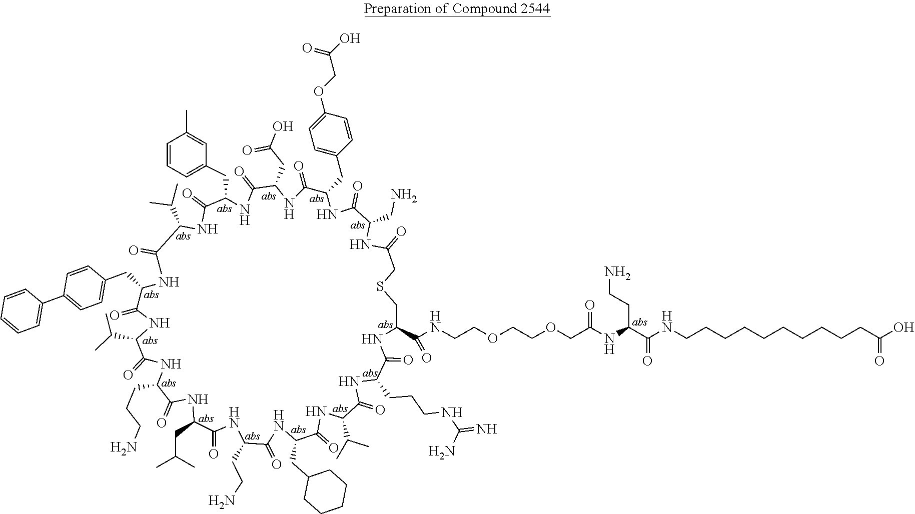 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2544 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.1 mg, and its estimated purity by LCMS analysis was 98.7%. Analysis condition B: Retention time=1.47 min; ESI-MS(+) m/z [M+2H]²⁺: 1166.2.

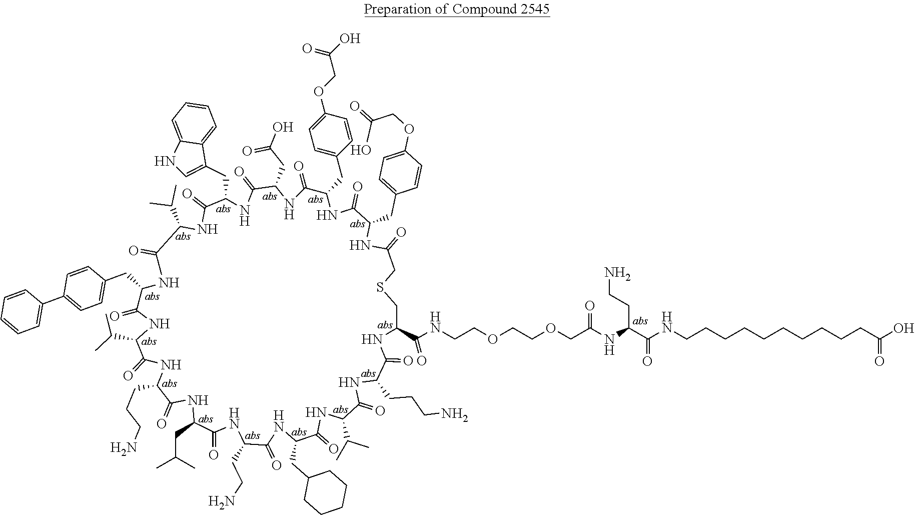 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2545 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 18.3 mg, and its estimated purity by LCMS analysis was 100%. Analysis condition A: Retention time=1.61 min; ESI-MS(+) m/z [M+2H]²⁺: 1225.1.

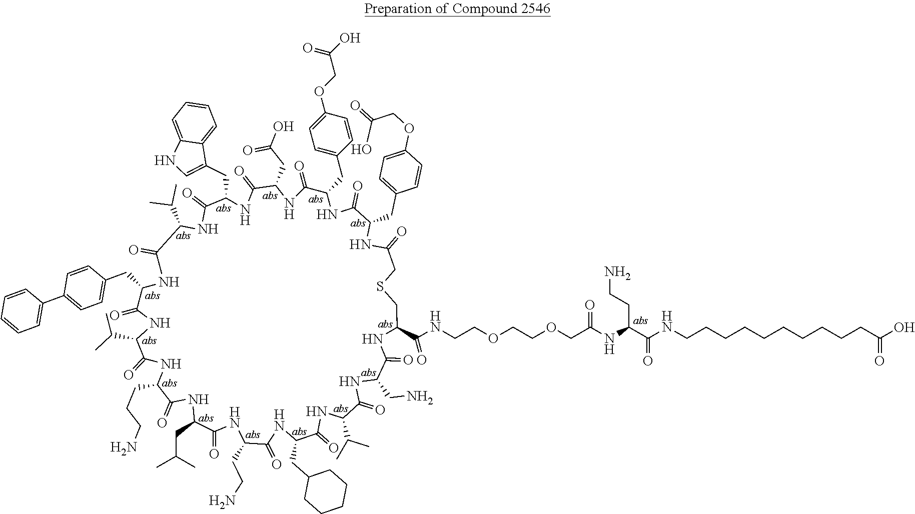 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2546 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 17.7 mg, and its estimated purity by LCMS analysis was 96.7%. Analysis condition A: Retention time=1.63, 1.7 min; ESI-MS(+) m/z [M+2H]²⁺: 1211.03, 1211.03.

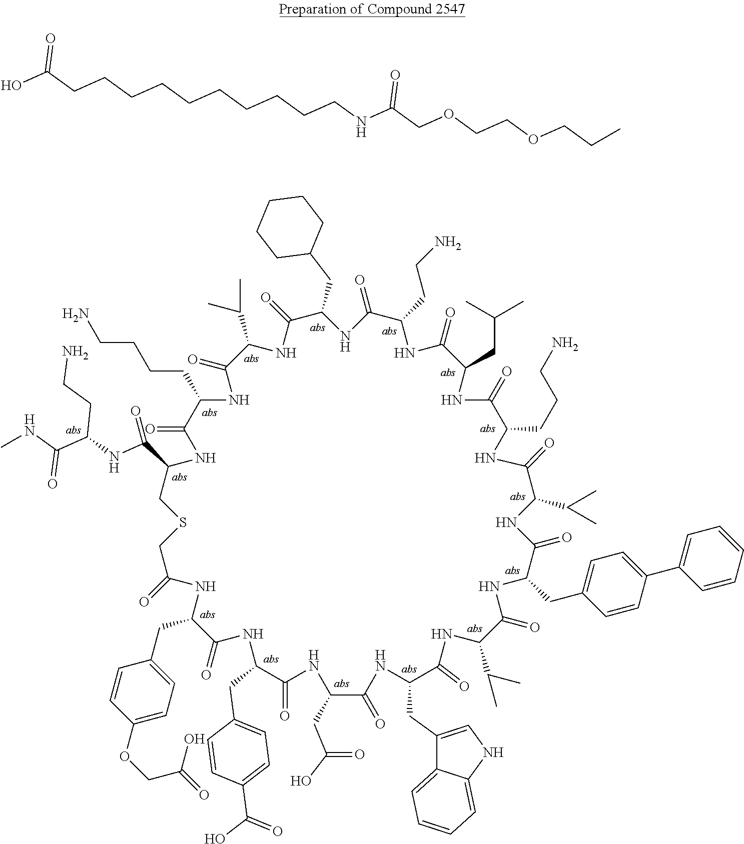

Compound 2547 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 13.5 mg, and its estimated purity by LCMS analysis was 100%. Analysis condition A: Retention time=1.57 min; ESI-MS(+) m/z [M+2H]²⁺: 1216.8.

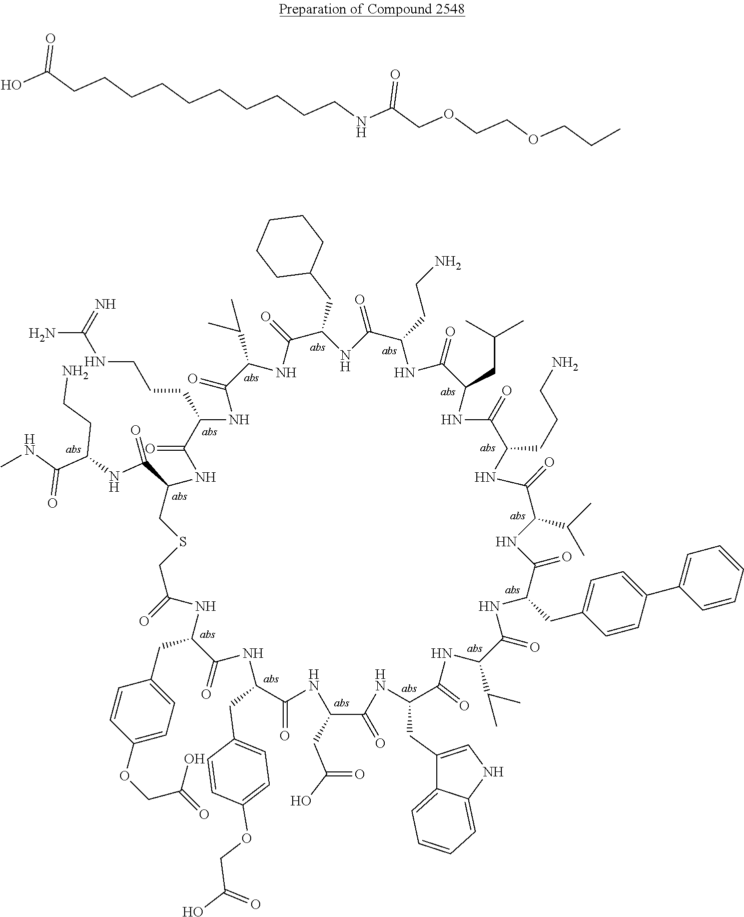

Compound 2548 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4.2 mg, and its estimated purity by LCMS analysis was 97.3%. Analysis condition A: Retention time=1.55 min; ESI-MS(+) m/z [M+3H]³⁺: 831.1.

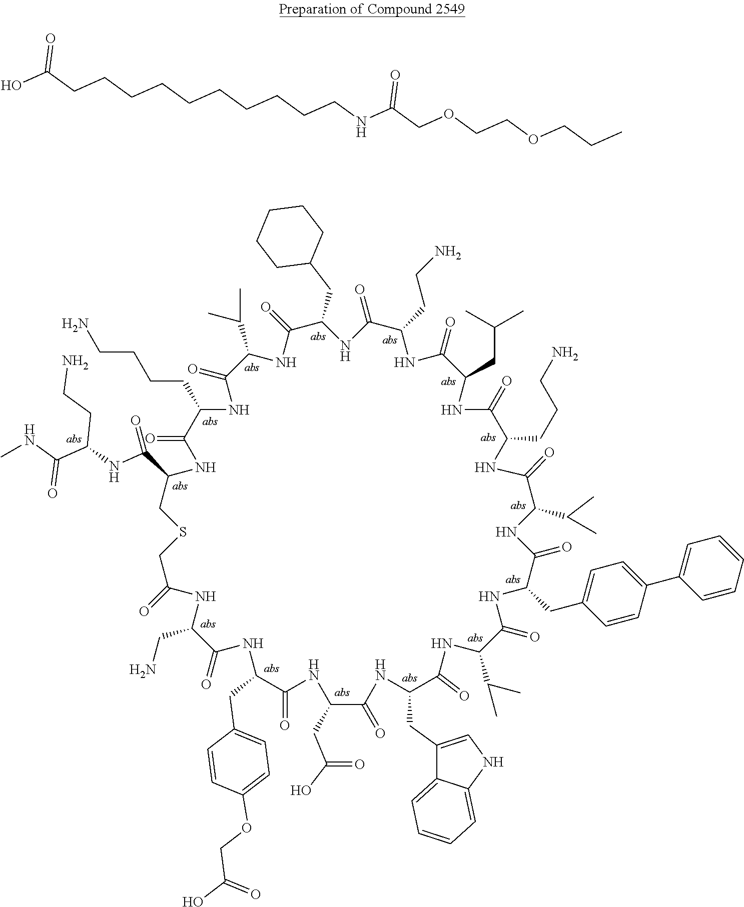

Compound 2549 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 7.9 mg, and its estimated purity by LCMS analysis was 99.1%. Analysis condition A: Retention time=1.41 min; ESI-MS(+) m/z [M+3H]³⁺: 776.7.

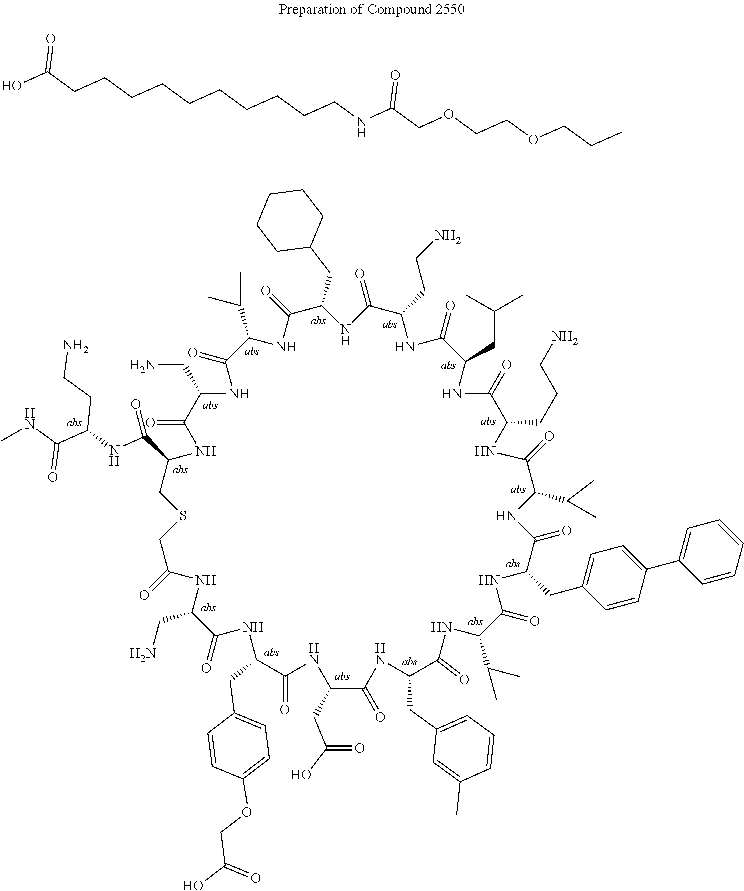

Compound 2550 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.3 mg, and its estimated purity by LCMS analysis was 99.2%. Analysis condition B: Retention time=1.59 min; ESI-MS(+) m/z [M+2H]²⁺: 1131.2.

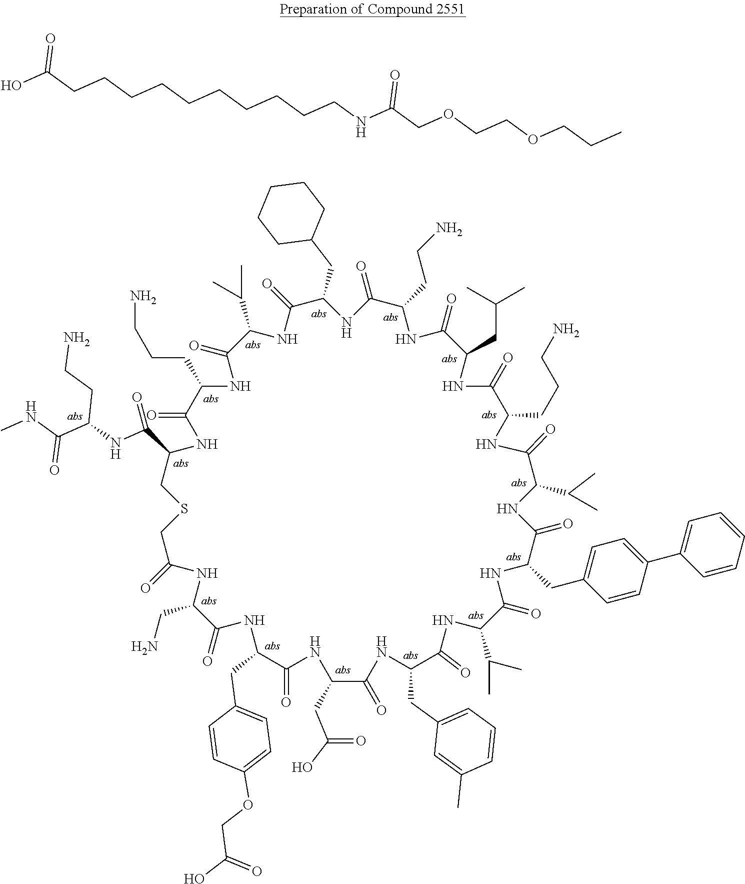

Compound 2551 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.9 mg, and its estimated purity by LCMS analysis was 93.4%. Analysis condition B: Retention time=1.55 min; ESI-MS(+) m/z [M+2H]²⁺: 1145.2.

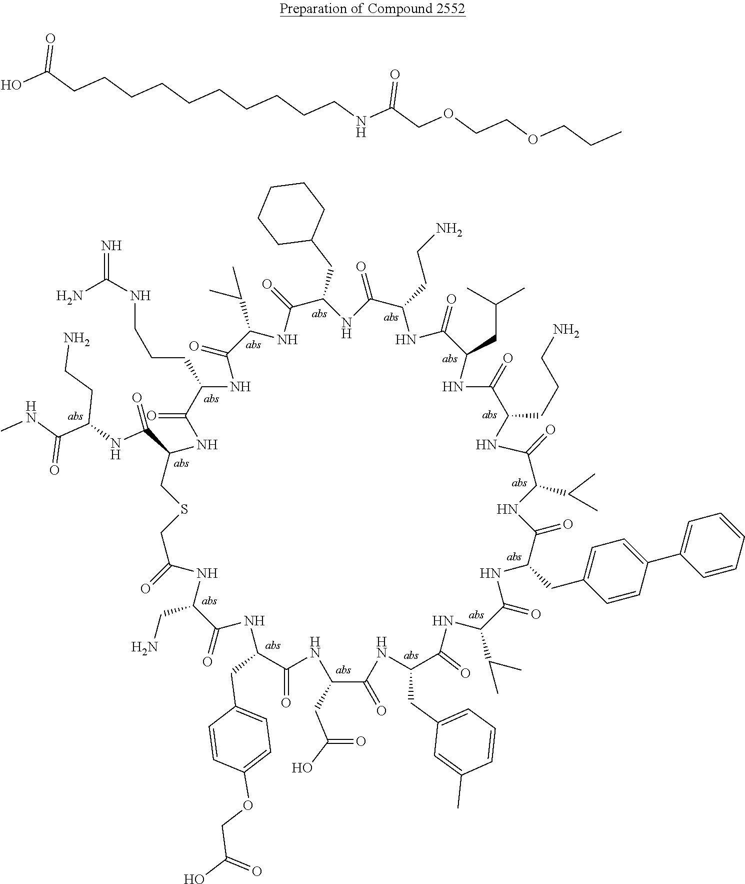

Compound 2552 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.6 mg, and its estimated purity by LCMS analysis was 93.3%. Analysis condition B: Retention time=1.53 min; ESI-MS(+) m/z [M+2H]²⁺: 1166.1.

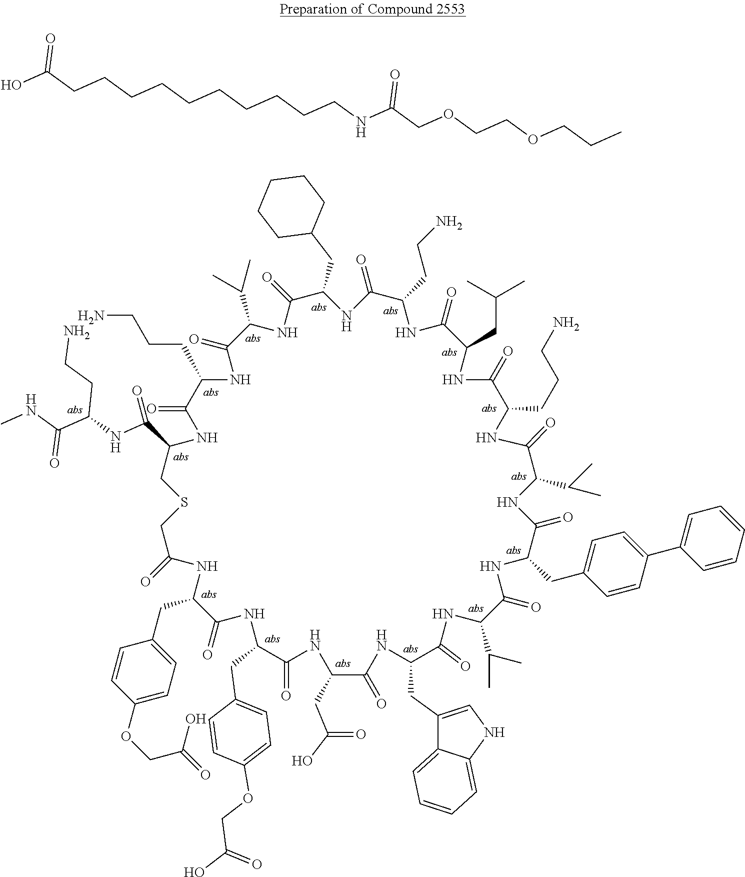

Compound 2553 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.7 mg, and its estimated purity by LCMS analysis was 97.2%. Analysis condition A: Retention time=1.57 min; ESI-MS(+) m/z [M+2H]²⁺: 1225.2.

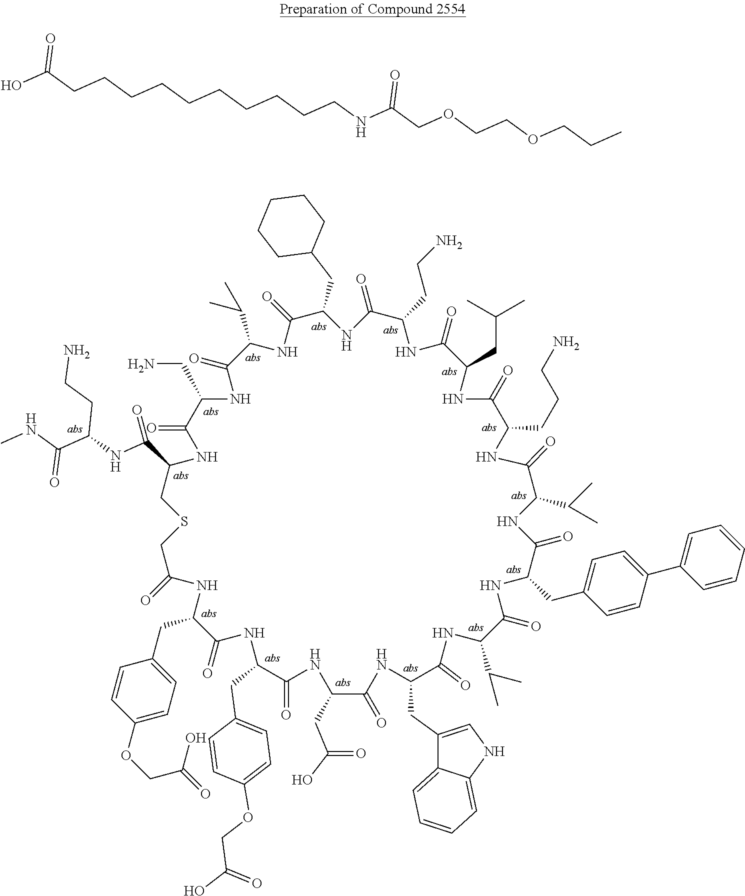

Compound 2554 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 25% B, 25-65% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 6.9 mg, and its estimated purity by LCMS analysis was 94.8%. Analysis condition A: Retention time=1.61 min; ESI-MS(+) m/z [M+2H]²⁺: 1211.1.

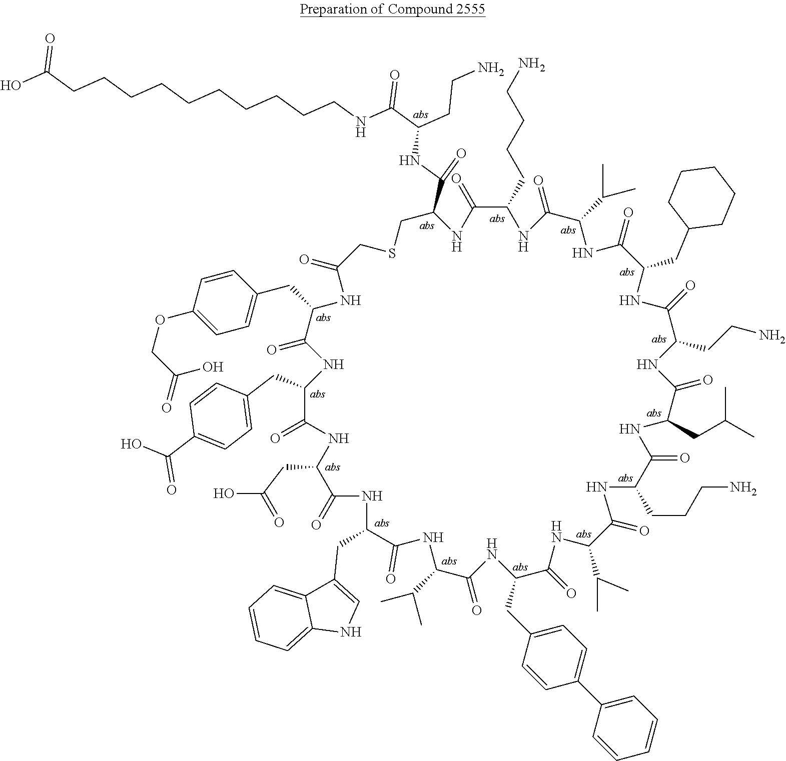 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2555 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.5 mg, and its estimated purity by LCMS analysis was 86.5%. Analysis condition A: Retention time=1.56 min; ESI-MS(+) m/z [M+3H]³⁺: 763.2.

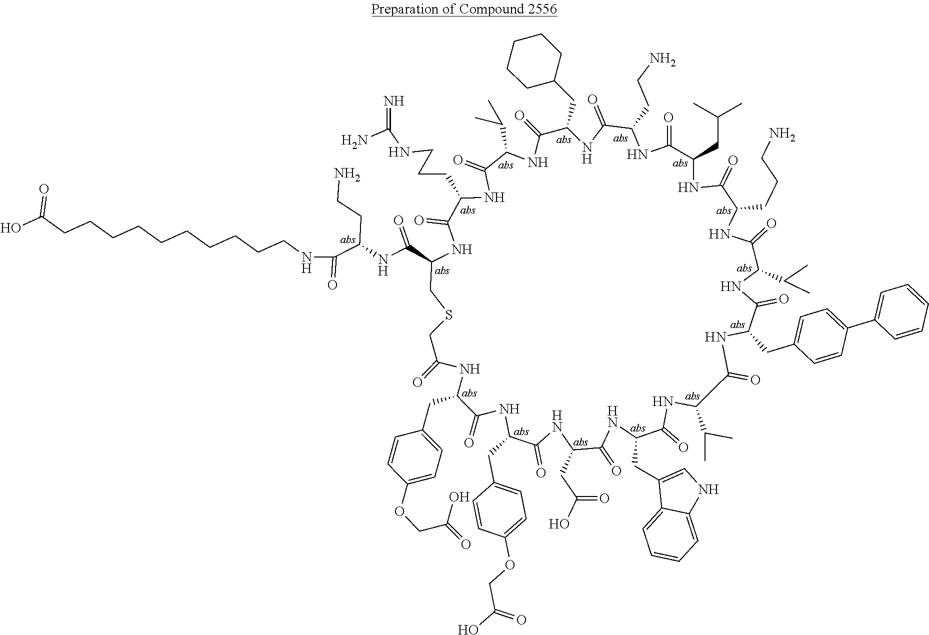 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2556 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.3 mg, and its estimated purity by LCMS analysis was 100%. Analysis condition A: Retention time=1.58 min; ESI-MS(+) m/z [M+2H]²⁺: 1173.8.

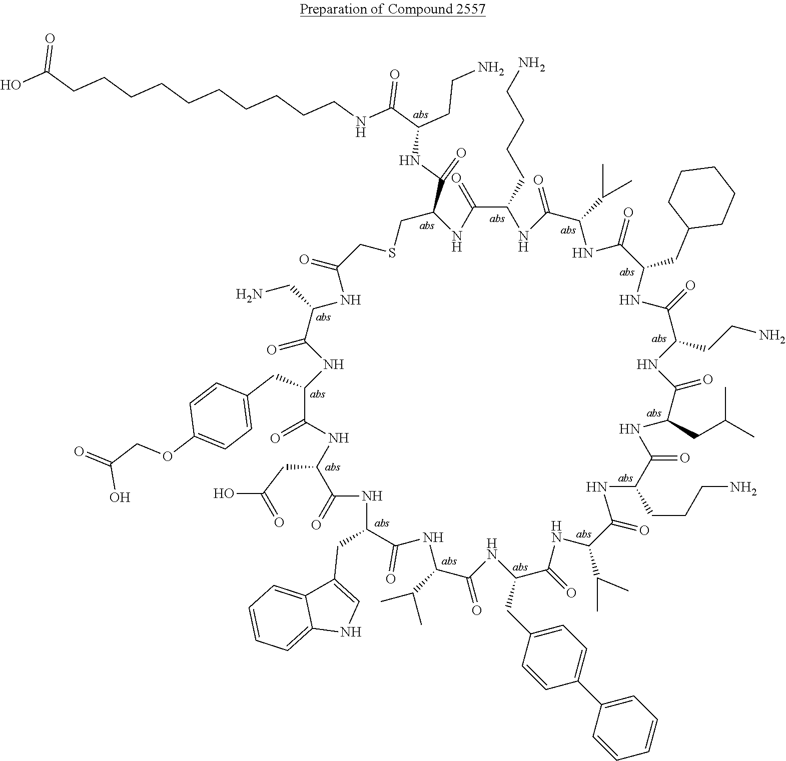 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2557 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.2 mg, and its estimated purity by LCMS analysis was 100%. Analysis condition A: Retention time=1.74 min; ESI-MS(+) m/z [M+3H]³⁺: 728.4.

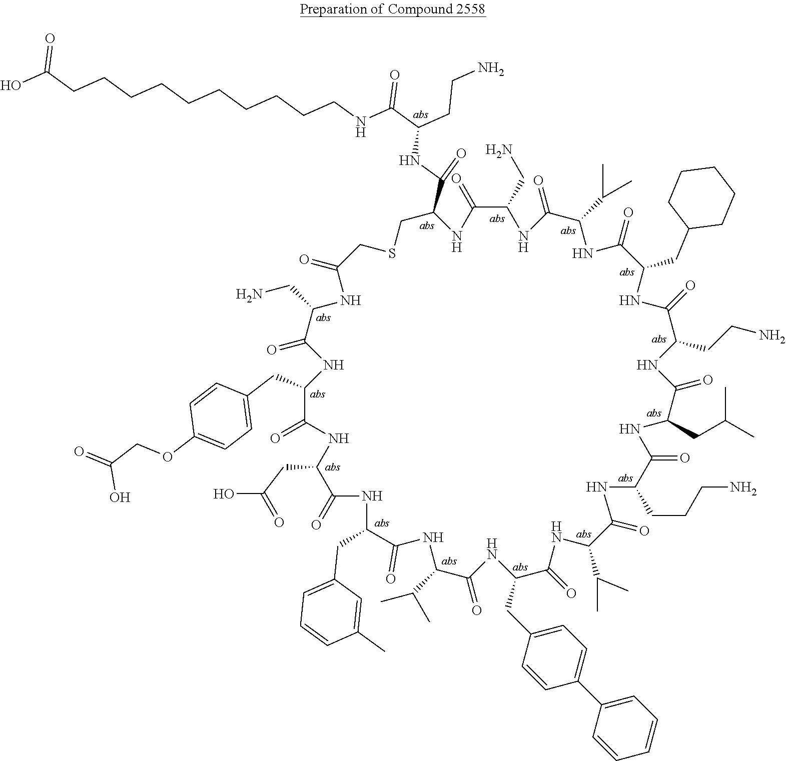 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2558 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 26% B, 26-66% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.5 mg, and its estimated purity by LCMS analysis was 93%. Analysis condition B: Retention time=1.45 min; ESI-MS(+) m/z [M+3H]³⁺: 705.9.

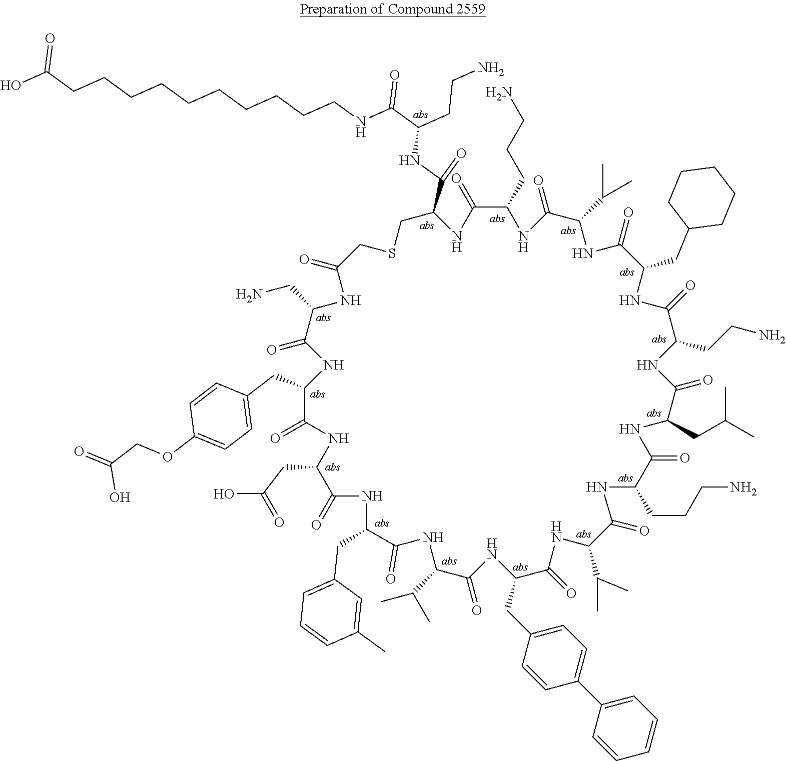 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2559 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.6 mg, and its estimated purity by LCMS analysis was 91.8%. Analysis condition B: Retention time=1.43 min; ESI-MS(+) m/z [M+2H]²⁺: 1073.

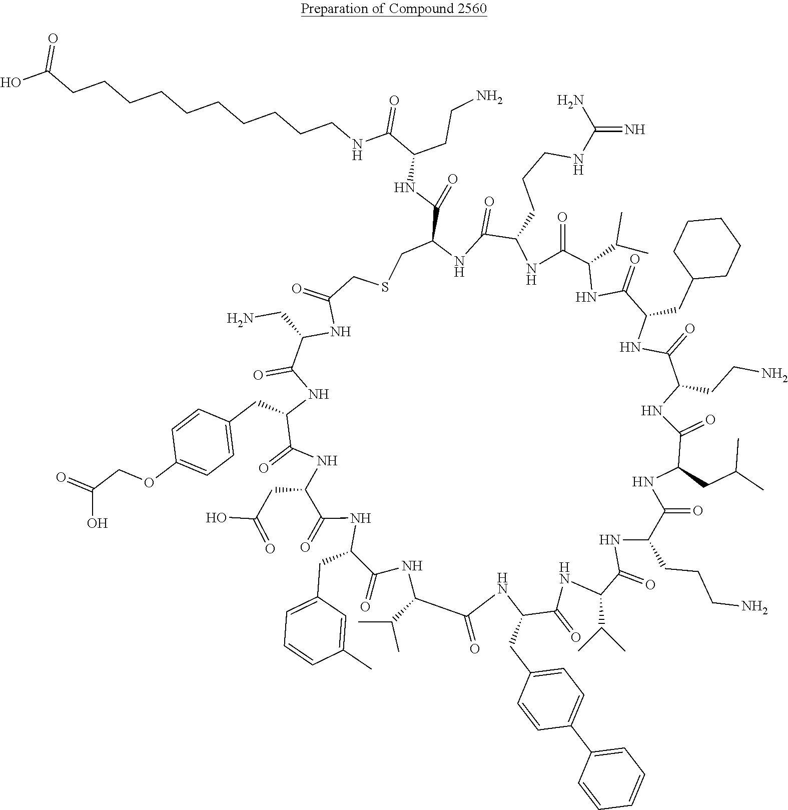 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2560 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 7.9 mg, and its estimated purity by LCMS analysis was 100%. Analysis condition A: Retention time=1.78 min; ESI-MS(+) m/z [M+3H]³⁺: 729.1.

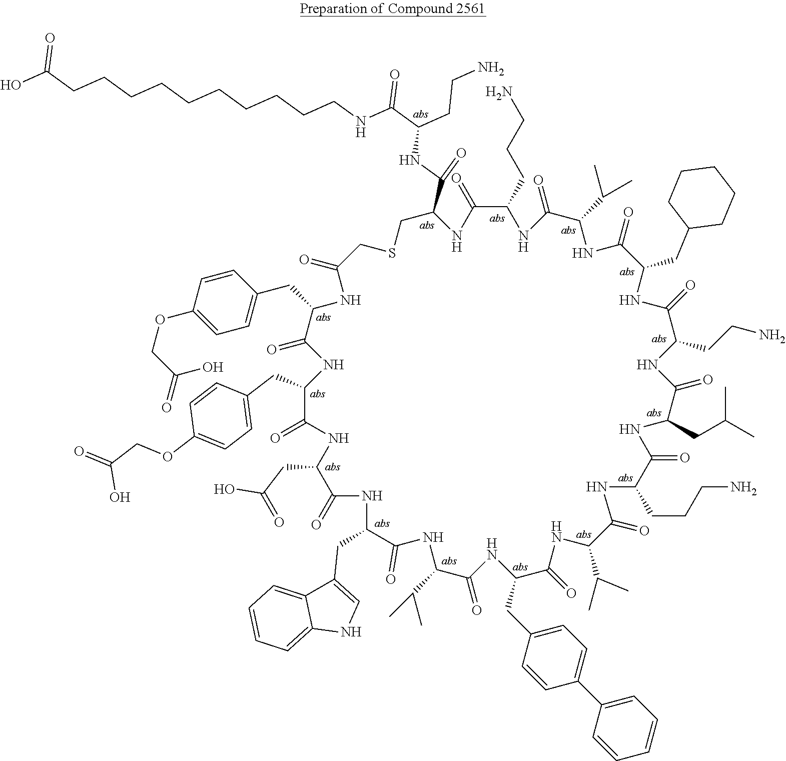 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2561 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 2-minute hold at 100% B; Flow Rate: 45 mL/min; Column Temperature: 25 C. Fraction collection was triggered by MS signals. Fractions containing the desired product were combined and dried via centrifugal evaporation. The yield of the product was 4.1 mg, and its estimated purity by LCMS analysis was 91.6%. Analysis condition A: Retention time=1.56 min; ESI-MS(+) m/z [M+2H]²⁺: 1153.

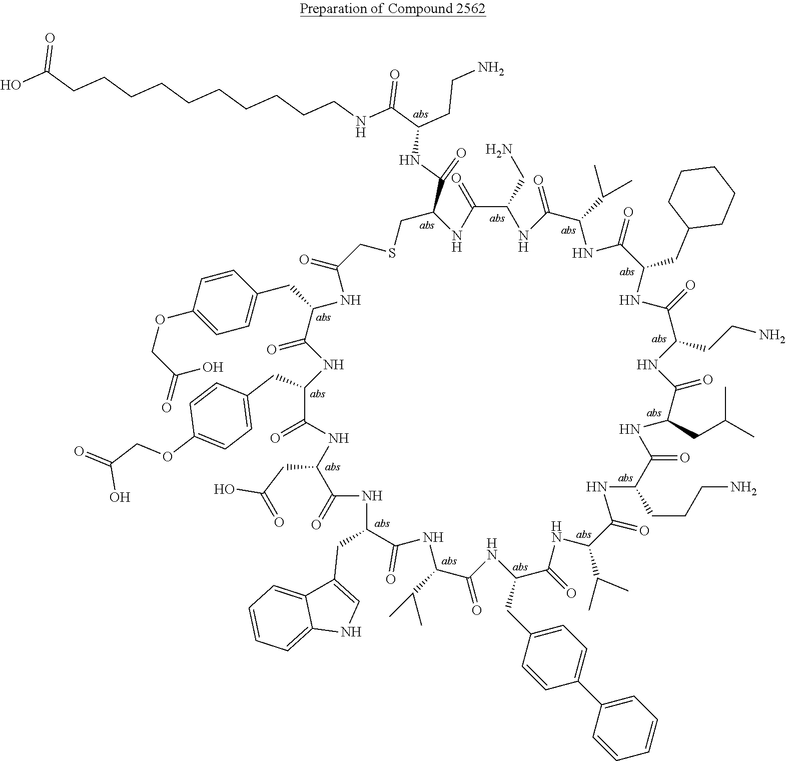 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2562 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 24% B, 24-64% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 0.9 mg, and its estimated purity by LCMS analysis was 87.8%. Analysis condition B: Retention time=1.55 min; ESI-MS(+) m/z [M+3H]³⁺: 759.3.

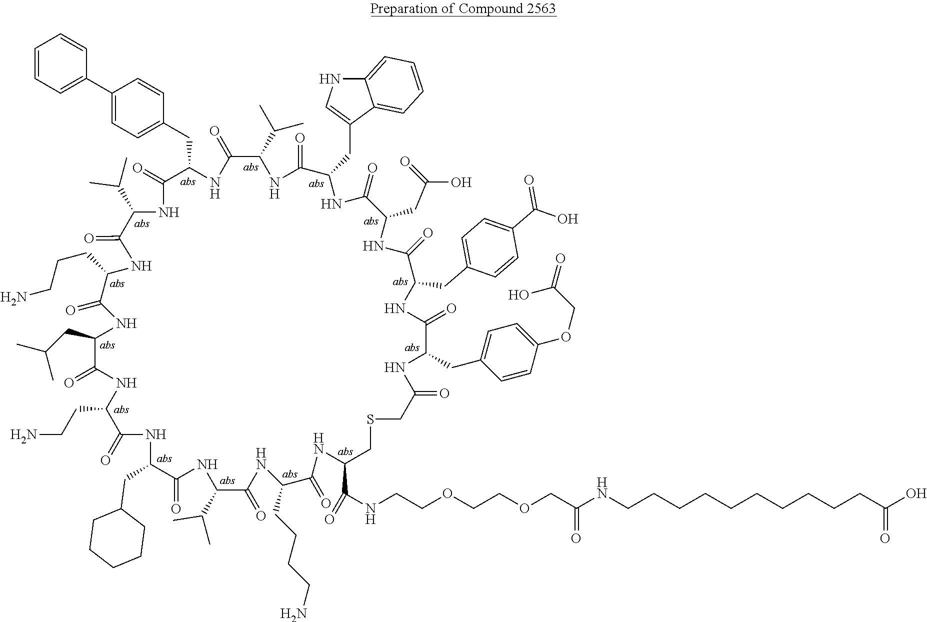 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2563 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 10.4 mg, and its estimated purity by LCMS analysis was 91.7%. Analysis condition B: Retention time=1.69 min; ESI-MS(+) m/z [M+2H]²⁺: 1167.

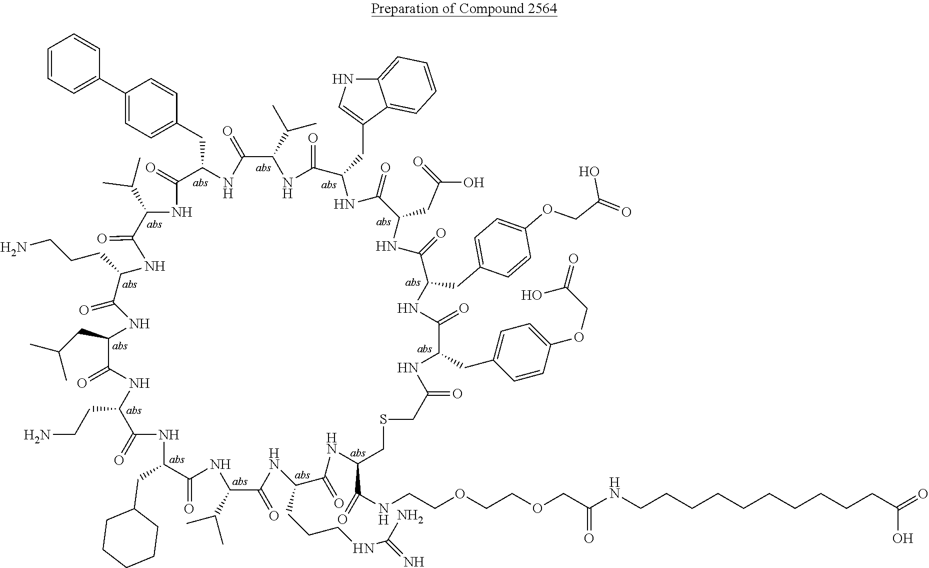 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2564 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 16.8 mg, and its estimated purity by LCMS analysis was 92.5%. Analysis condition A: Retention time=1.59 min; ESI-MS(+) m/z [M+2H]²⁺: 1195.7.

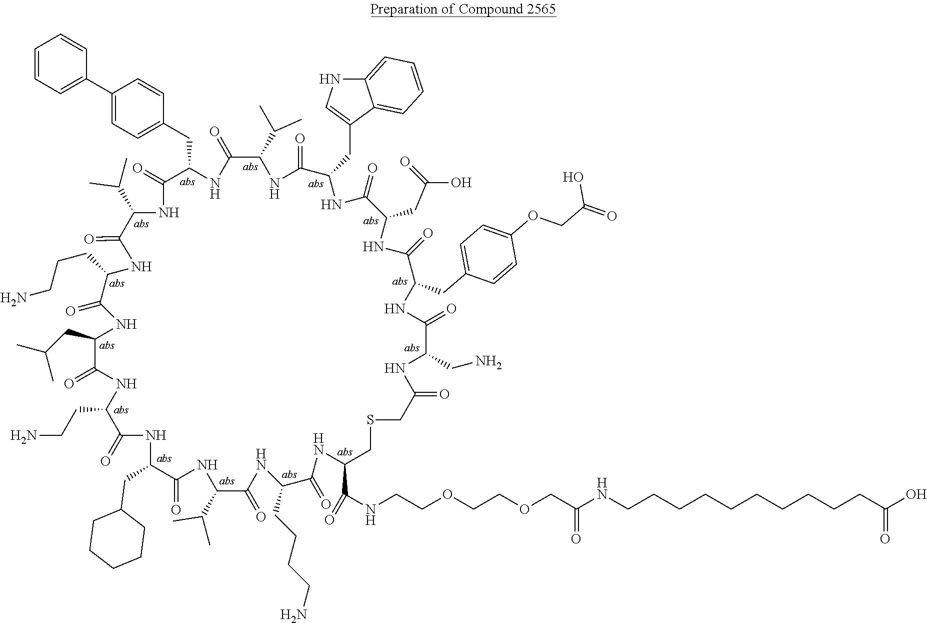 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2565 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-52% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 7 mg, and its estimated purity by LCMS analysis was 94.6%. Analysis condition A: Retention time=1.57 min; ESI-MS(+) m/z [M+3H]³⁺: 743.

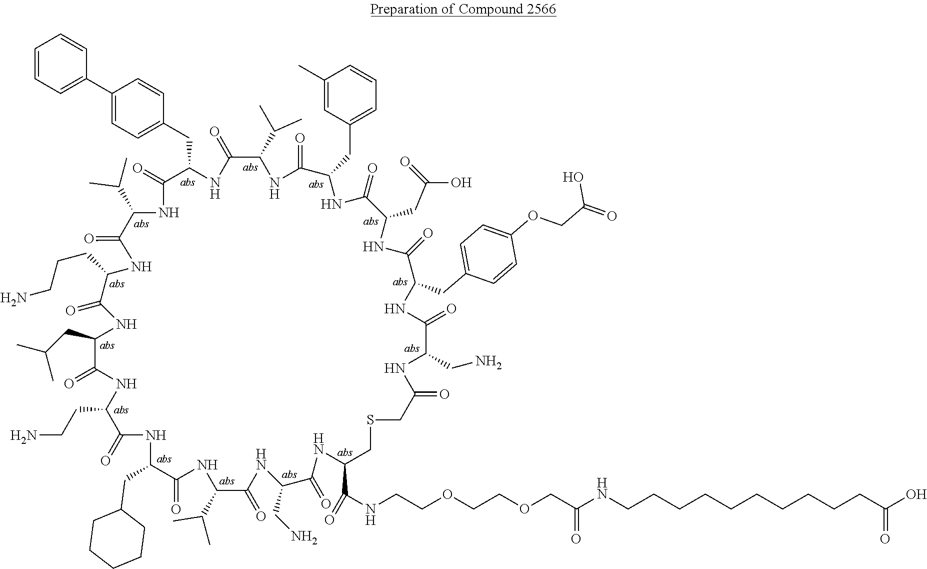 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2566 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 23 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.1 mg, and its estimated purity by LCMS analysis was 100%. Analysis condition A: Retention time=1.7 min; ESI-MS(+) m/z [M+2H]²⁺: 1081.2.

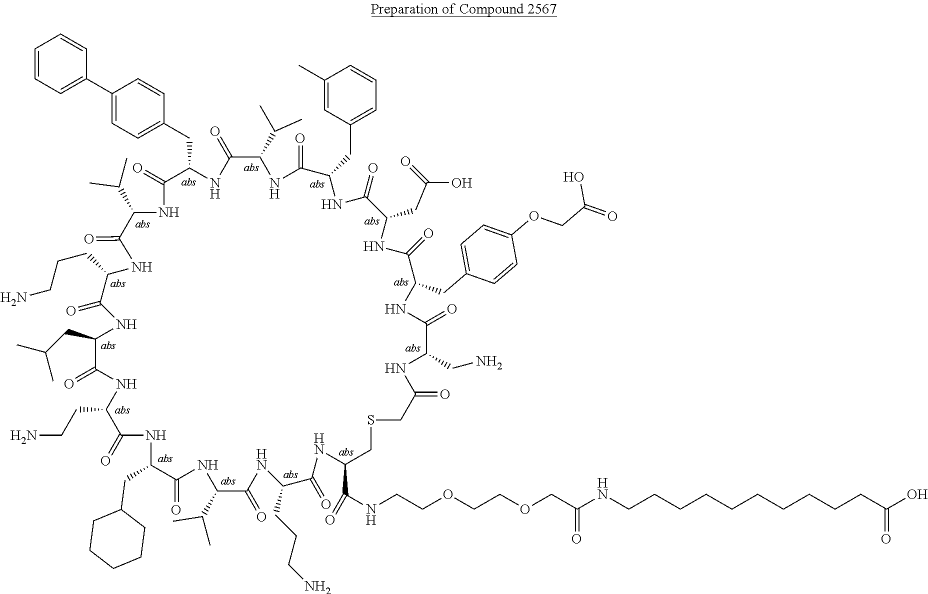 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2567 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.8 mg, and its estimated purity by LCMS analysis was 95.6%. Analysis condition A: Retention time=1.51 min; ESI-MS(+) m/z [M+2H]²⁺: 730.2.

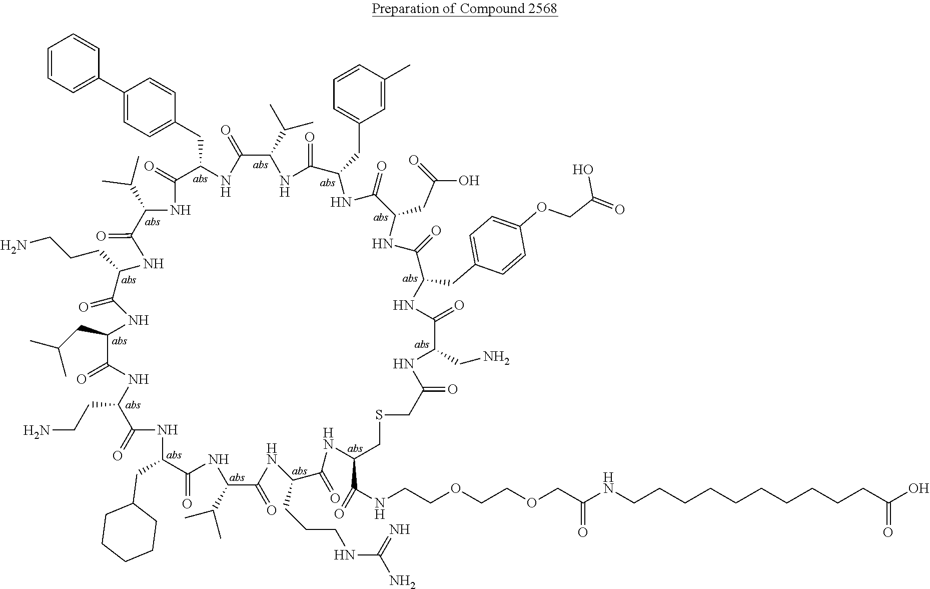 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2568 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 8.6 mg, and its estimated purity by LCMS analysis was 97.3%. Analysis condition A: Retention time=1.58 min; ESI-MS(+) m/z [M+2H]²⁺: 1116.1.

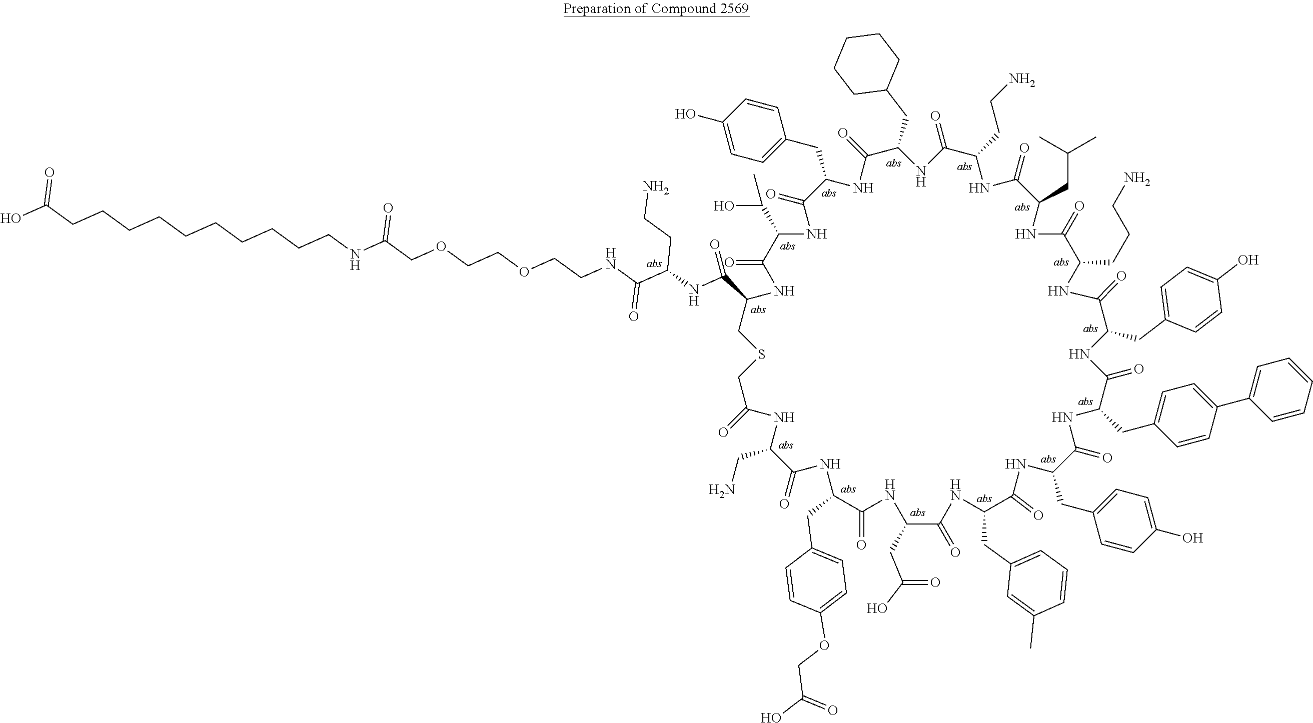 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2569 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 47.6 mg, and its estimated purity by LCMS analysis was 98.3%. Analysis condition A: Retention time=1.6 min; ESI-MS(+) m/z [M+2H]²⁺: 1235.1.

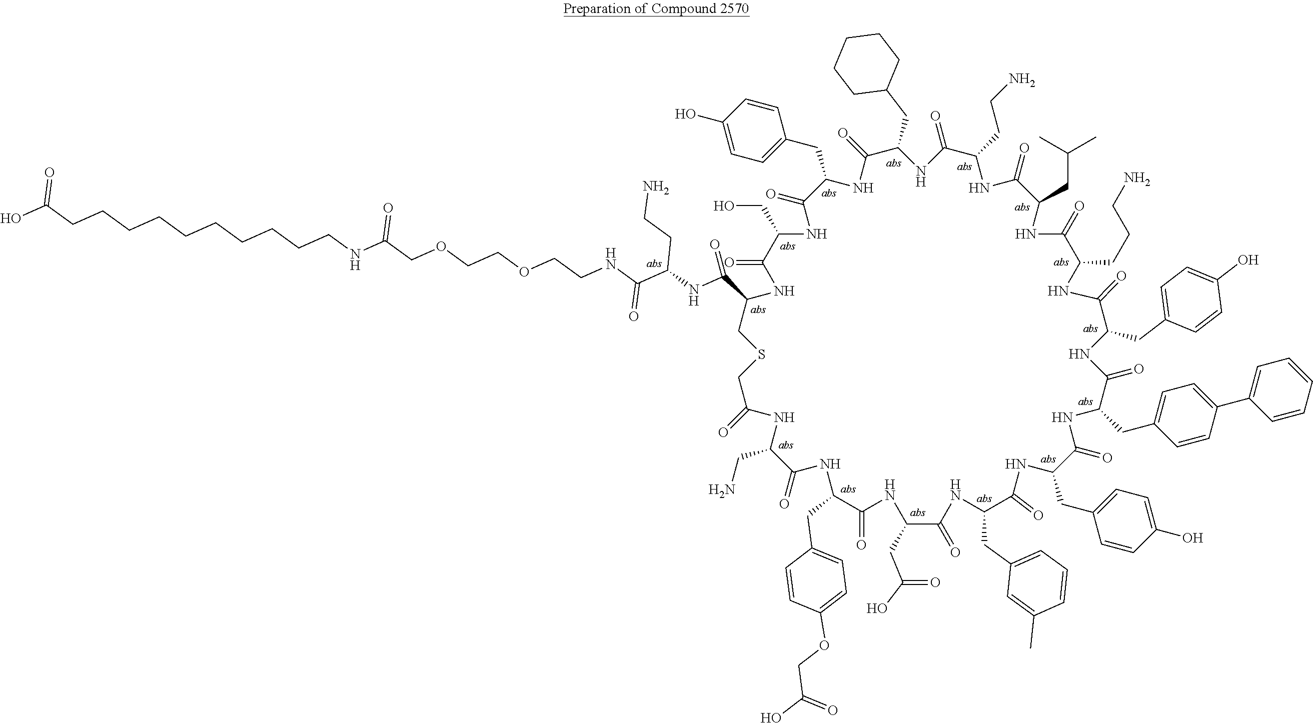 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2570 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 15.2 mg, and its estimated purity by LCMS analysis was 97.1%. Analysis condition A: Retention time=1.57 min; ESI-MS(+) m/z [M+2H]²⁺: 1227.2.

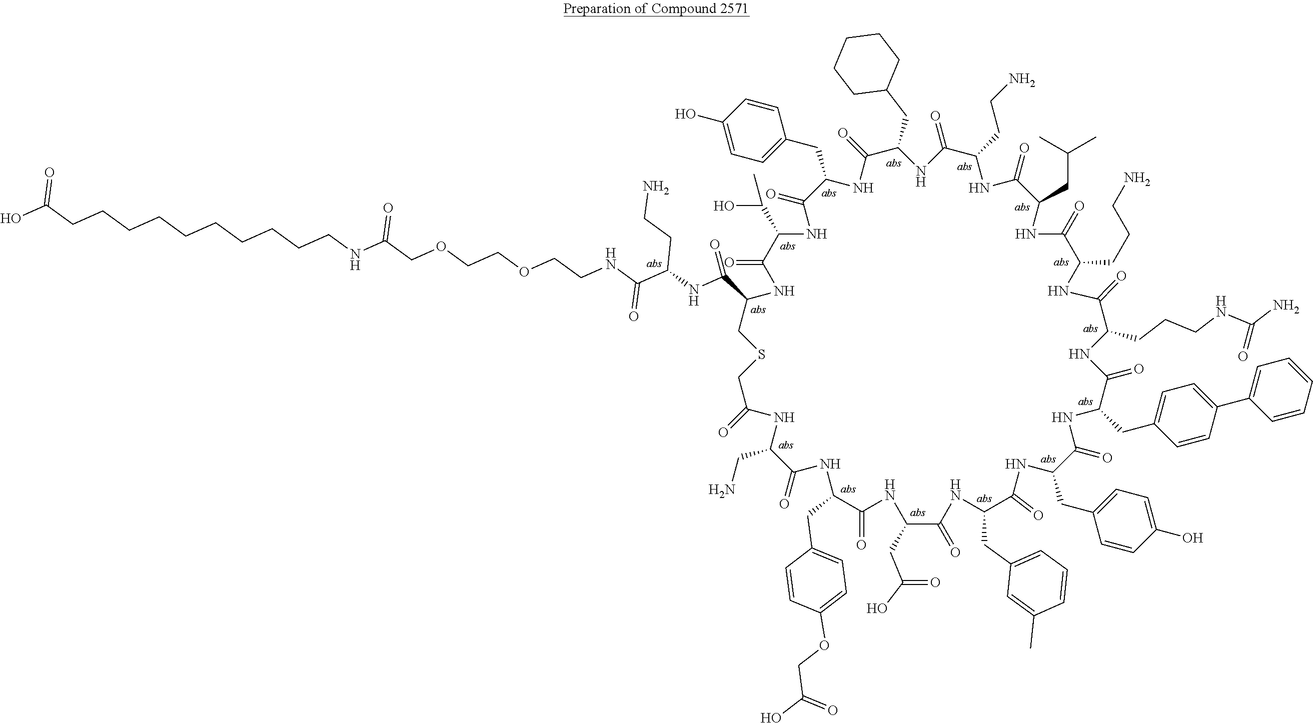 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(O)cc3)NC2=O)c1]

Compound 2571 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 72.2 mg, and its estimated purity by LCMS analysis was 92%. Analysis condition A: Retention time=1.53 min; ESI-MS(+) m/z [M+3H]³⁺: 821.4.

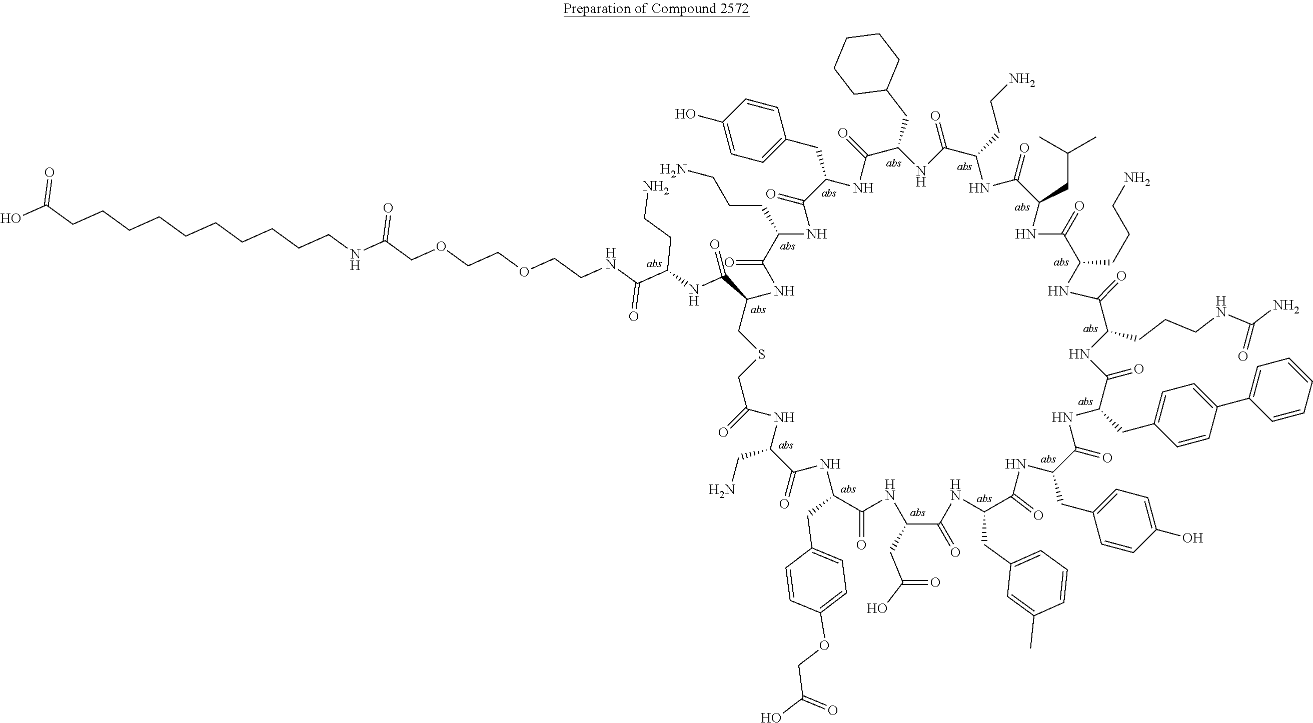 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(O)cc3)NC2=O)c1]

Compound 2572 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.6 mg, and its estimated purity by LCMS analysis was 96.6%. Analysis condition A: Retention time=1.47 min; ESI-MS(+) m/z [M+2H]²⁺: 1238.1.

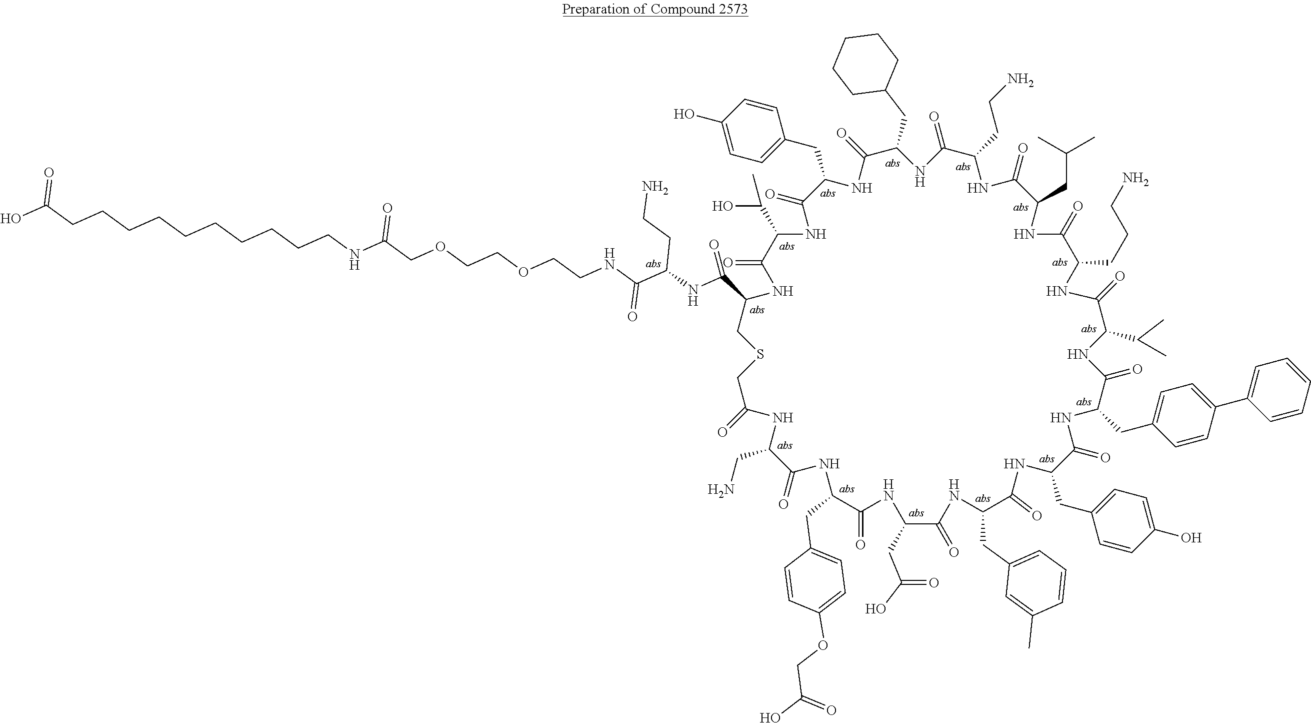 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](Cc3ccc(O)cc3)NC2=O)c1]

Compound 2573 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.3 mg, and its estimated purity by LCMS analysis was 93.7%. Analysis condition B: Retention time=1.61 min; ESI-MS(+) m/z [M+3H]³⁺: 802.

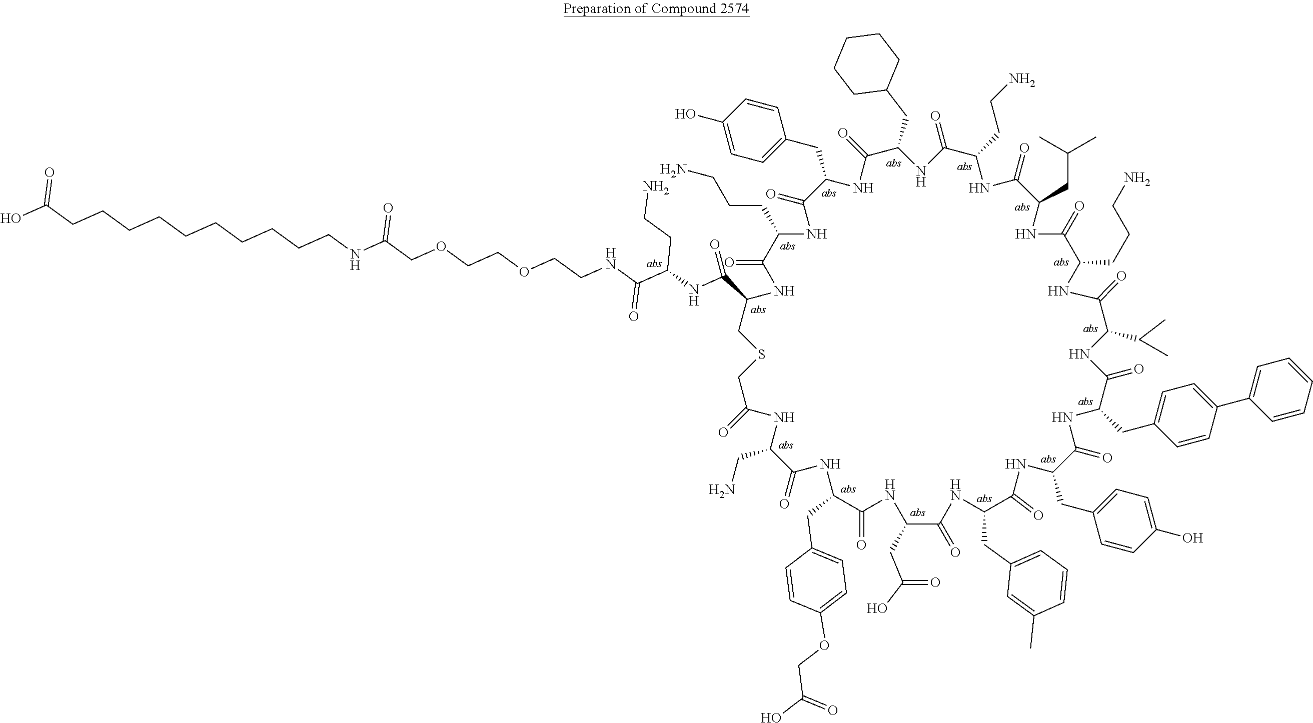 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](Cc3ccc(O)cc3)NC2=O)c1]

Compound 2574 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 18.3 mg, and its estimated purity by LCMS analysis was 100%. Analysis condition A: Retention time=1.7 min; ESI-MS(+) m/z [M+2H]²⁺: 1209.

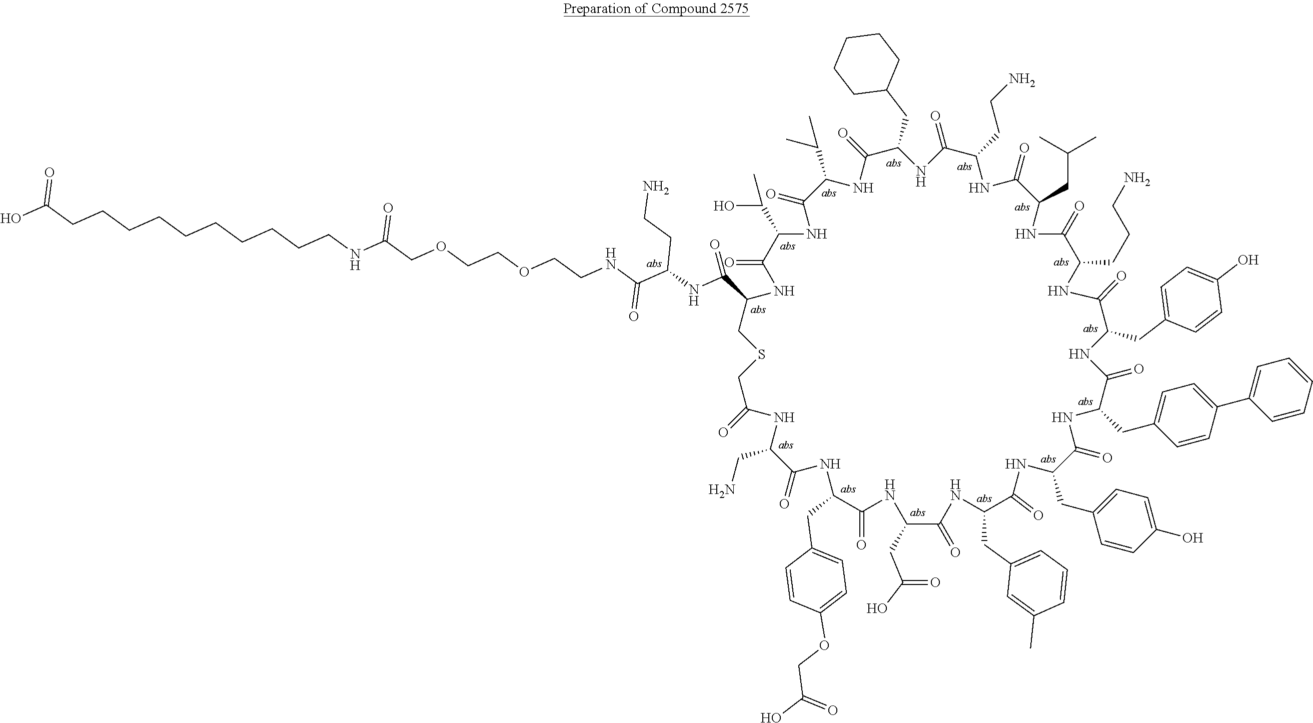 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2575 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20.7 mg, and its estimated purity by LCMS analysis was 92.7%. Analysis condition A: Retention time=1.6 min; ESI-MS(+) m/z [M+3H]³⁺: 802.2.

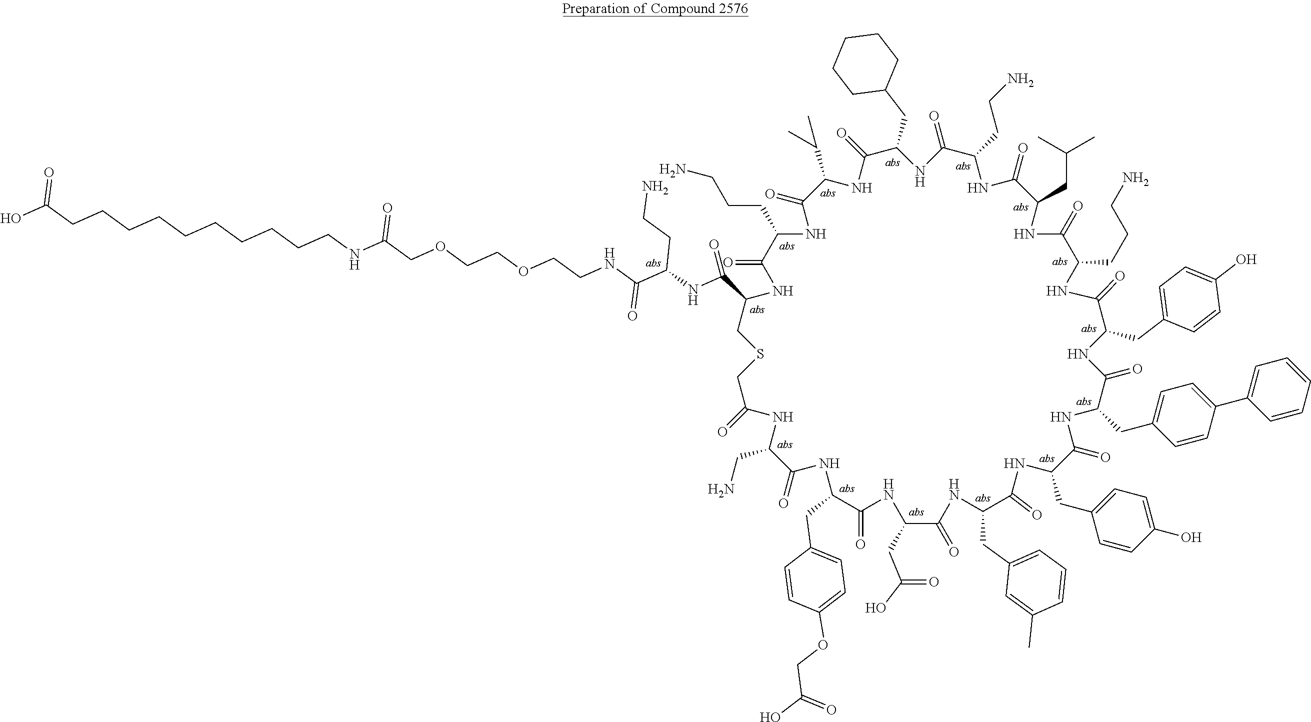 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2576 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 30.8 mg, and its estimated purity by LCMS analysis was 100%. Analysis condition A: Retention time=1.61 min; ESI-MS(+) m/z [M+2H]²⁺: 1209.

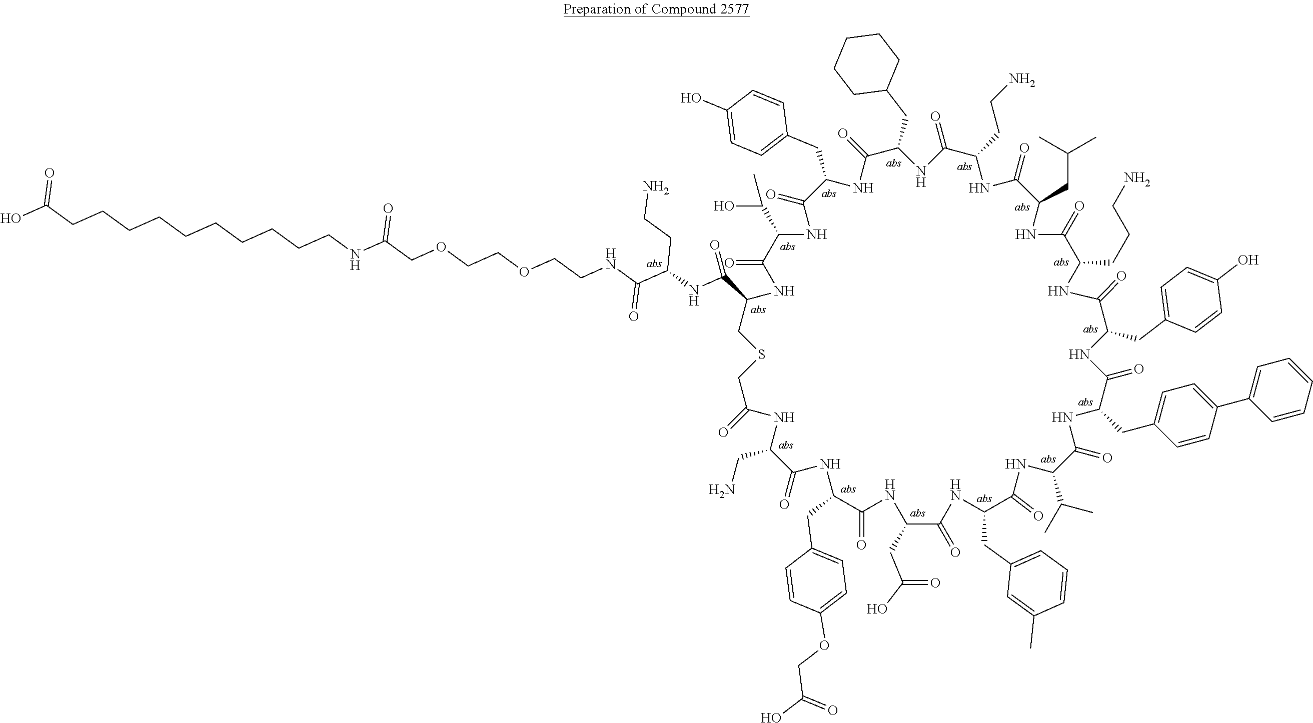 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2577 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 44.4 mg, and its estimated purity by LCMS analysis was 100%. Analysis condition A: Retention time=1.59 min; ESI-MS(+) m/z [M+3H]³⁺: 801.9.

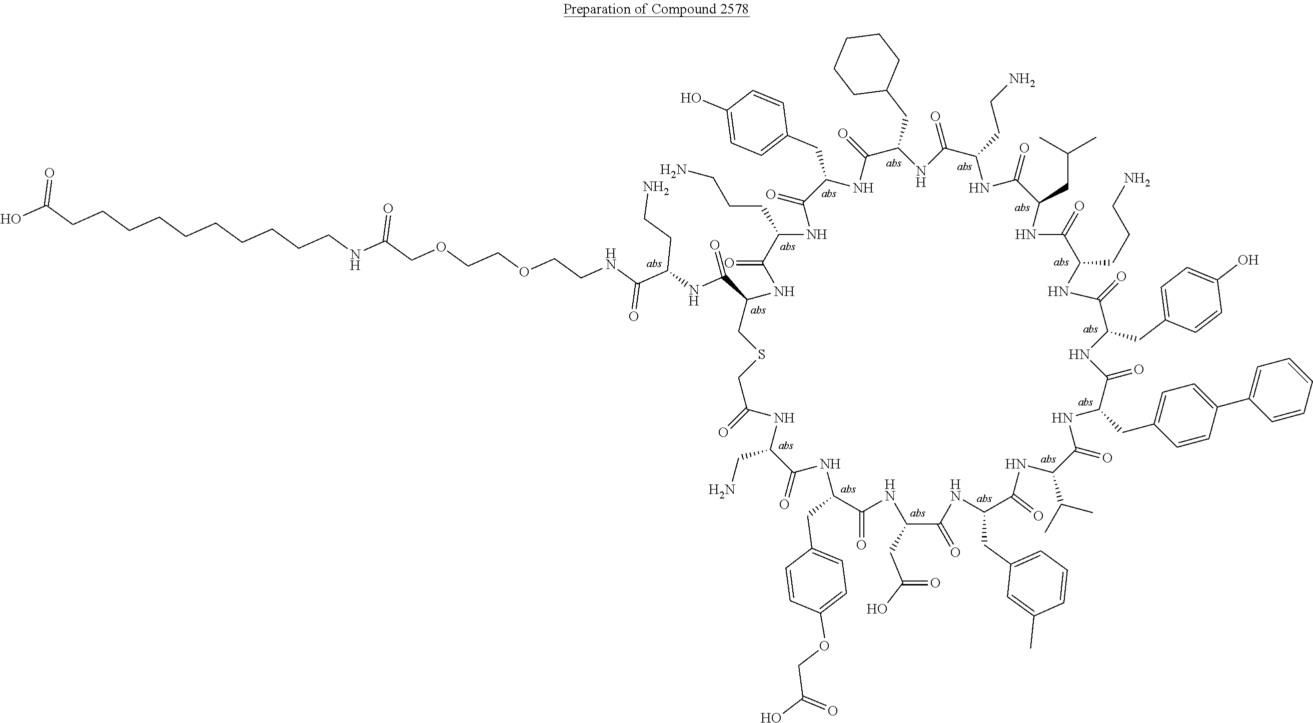 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2578 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.9 mg, and its estimated purity by LCMS analysis was 97.7%. Analysis condition A: Retention time=1.65 min; ESI-MS(+) m/z [M+3H]³⁺: 807.2.

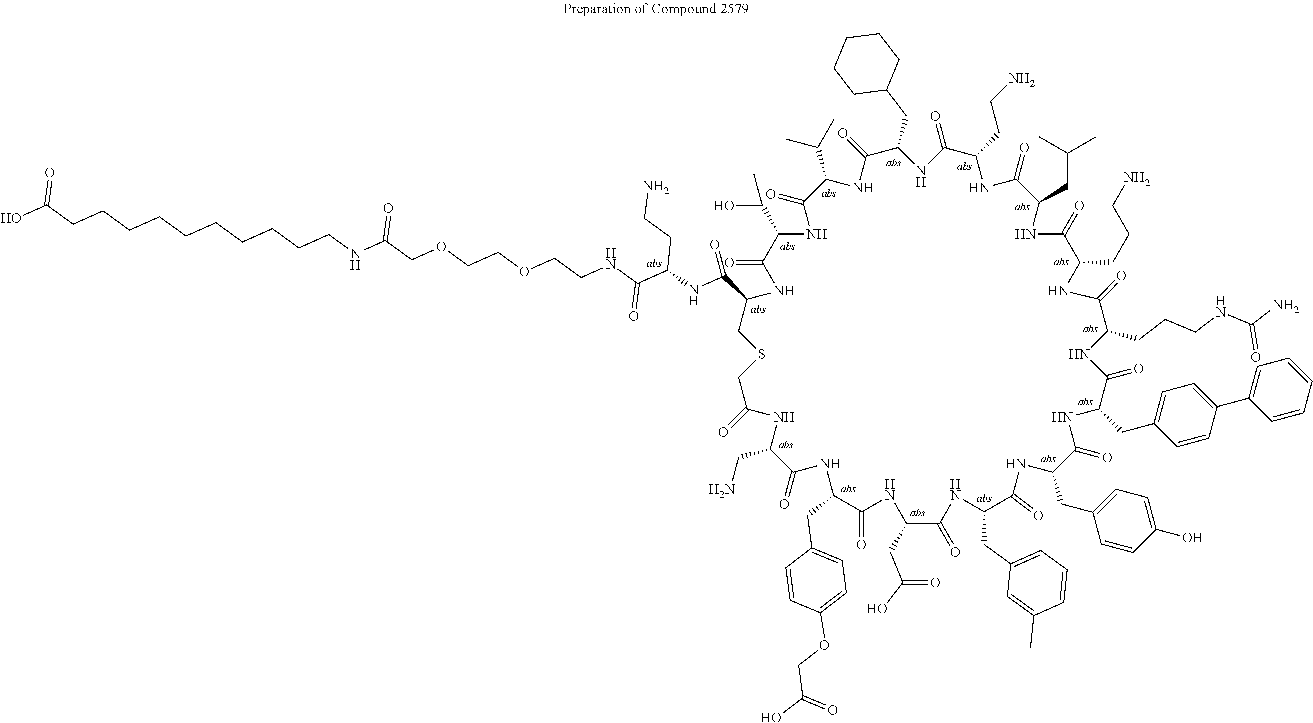 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(O)cc3)NC2=O)c1]

Compound 2579 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.6 mg, and its estimated purity by LCMS analysis was 92.8%. Analysis condition B: Retention time=1.6 min; ESI-MS(+) m/z [M+3H]³⁺: 800.

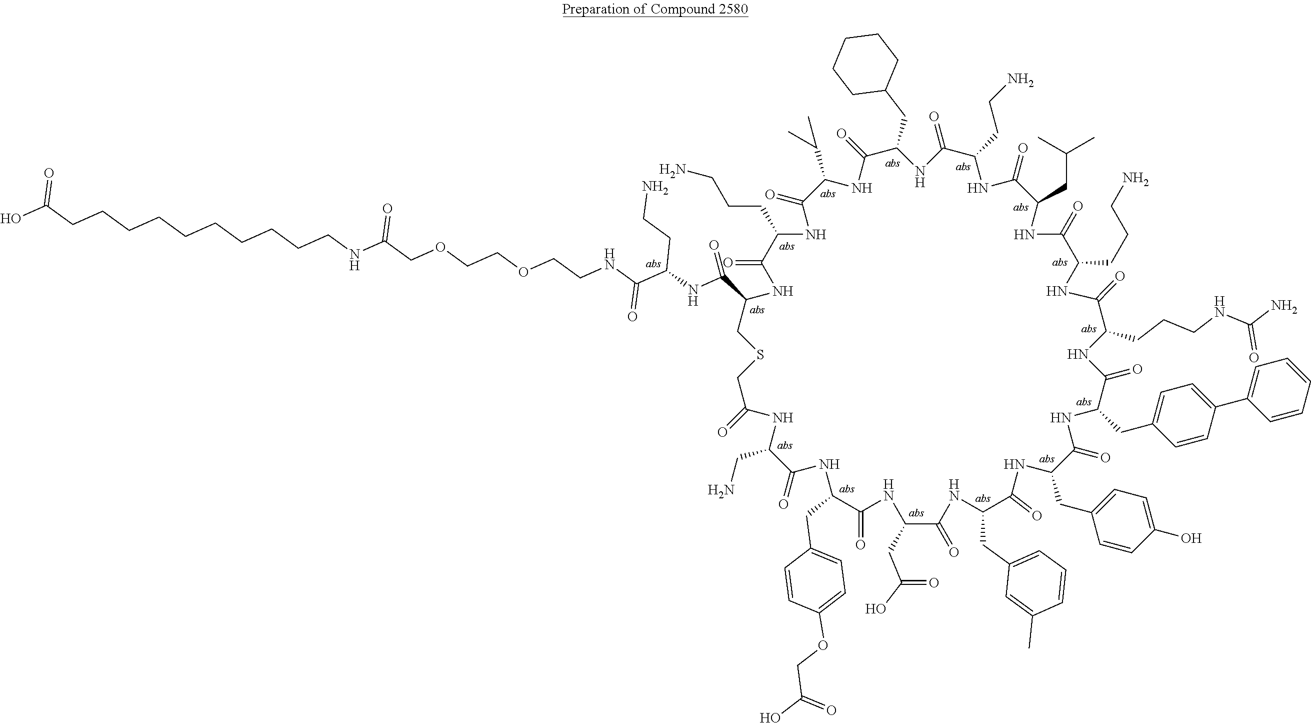 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(O)cc3)NC2=O)c1]

Compound 2580 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 19.6 mg, and its estimated purity by LCMS analysis was 100%. Analysis condition A: Retention time=1.46 min; ESI-MS(+) m/z [M+2H]²⁺: 1206.1.

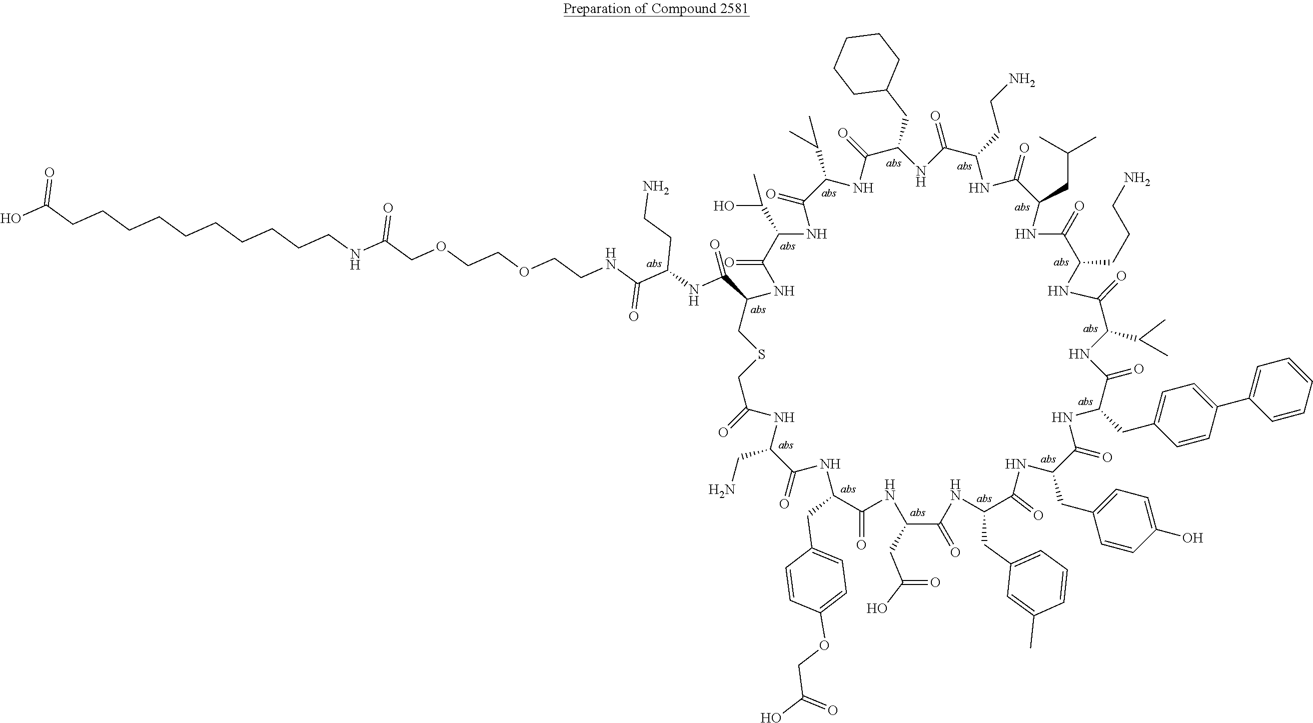 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](Cc3ccc(O)cc3)NC2=O)c1]

Compound 2581 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 22.7 mg, and its estimated purity by LCMS analysis was 95.2%. Analysis condition B: Retention time=1.6 min; ESI-MS(+) m/z [M+3H]³⁺: 781.1.

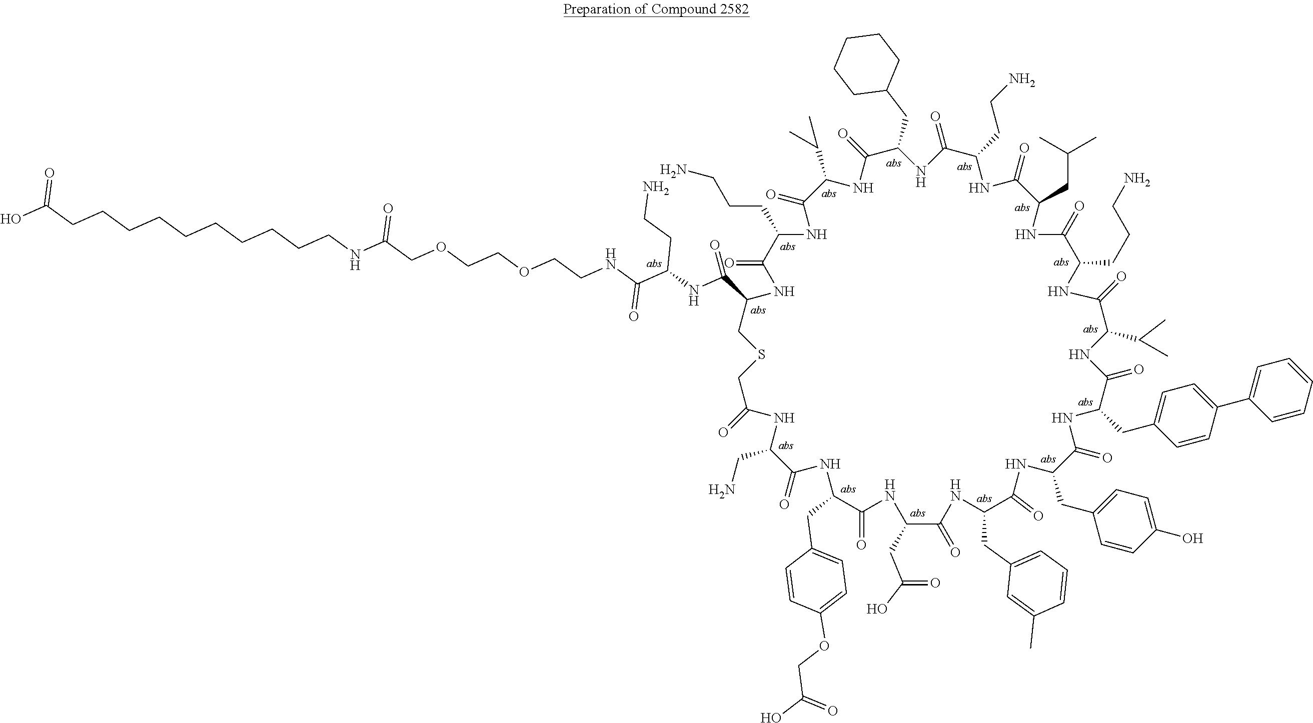 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](Cc3ccc(O)cc3)NC2=O)c1]

Compound 2582 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 22.3 mg, and its estimated purity by LCMS analysis was 95.6%. Analysis condition B: Retention time=1.57 min; ESI-MS(+) m/z [M+2H]²⁺: 1177.2.

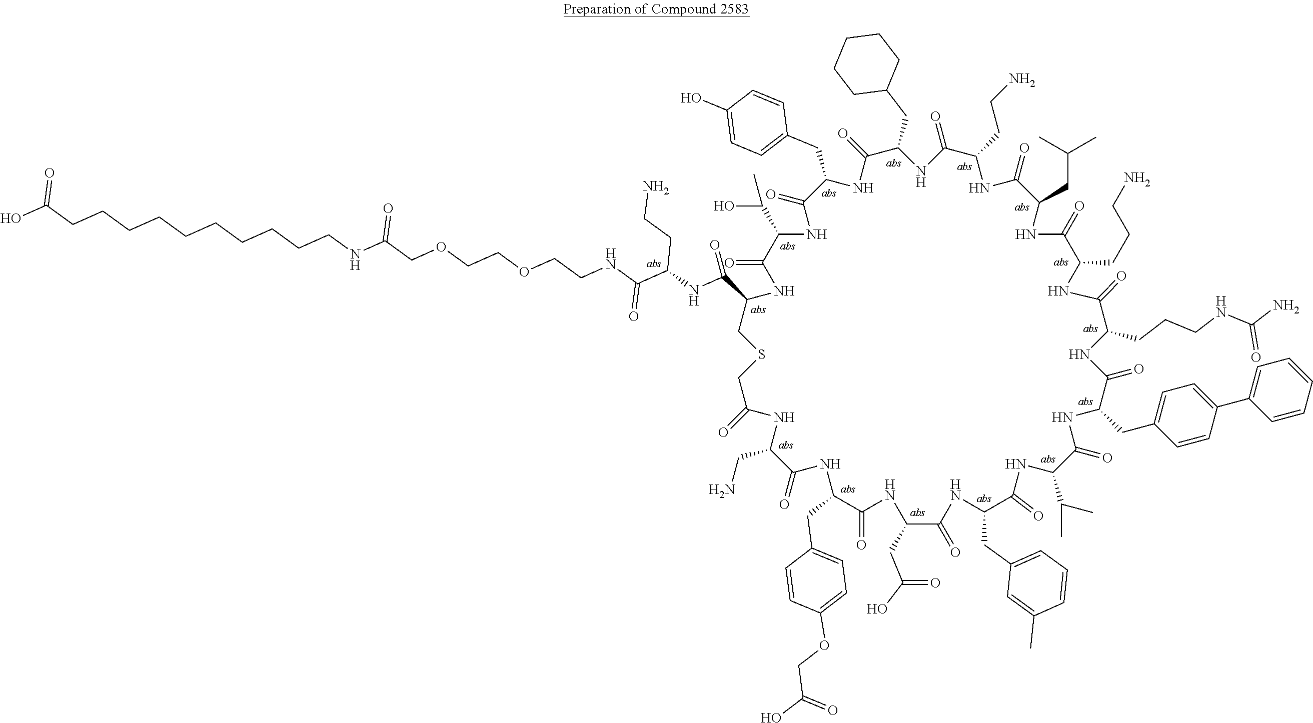 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2583 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 44 mg, and its estimated purity by LCMS analysis was 98.2%. Analysis condition B: Retention time=1.58 min; ESI-MS(+) m/z [M+2H]²⁺: 1199.4.

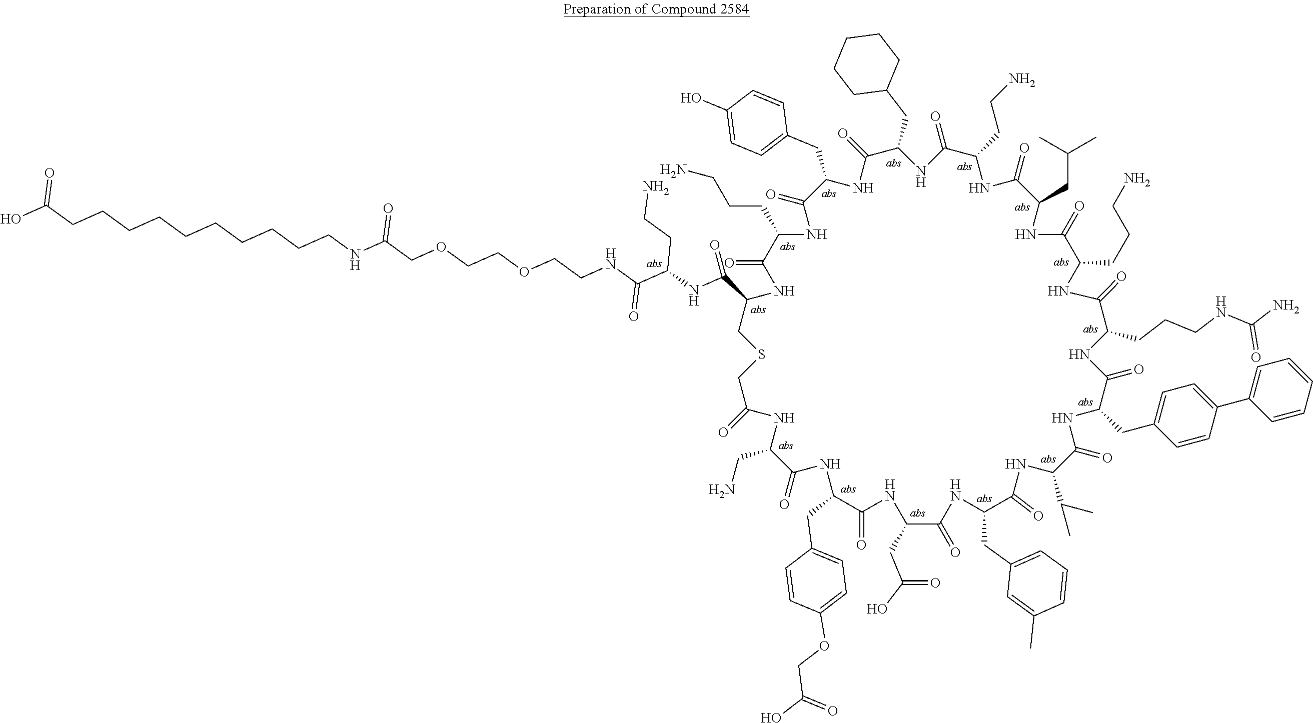 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2584 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.4 mg, and its estimated purity by LCMS analysis was 94.7%. Analysis condition A: Retention time=1.66 min; ESI-MS(+) m/z [M+3H]³⁺: 805.2.

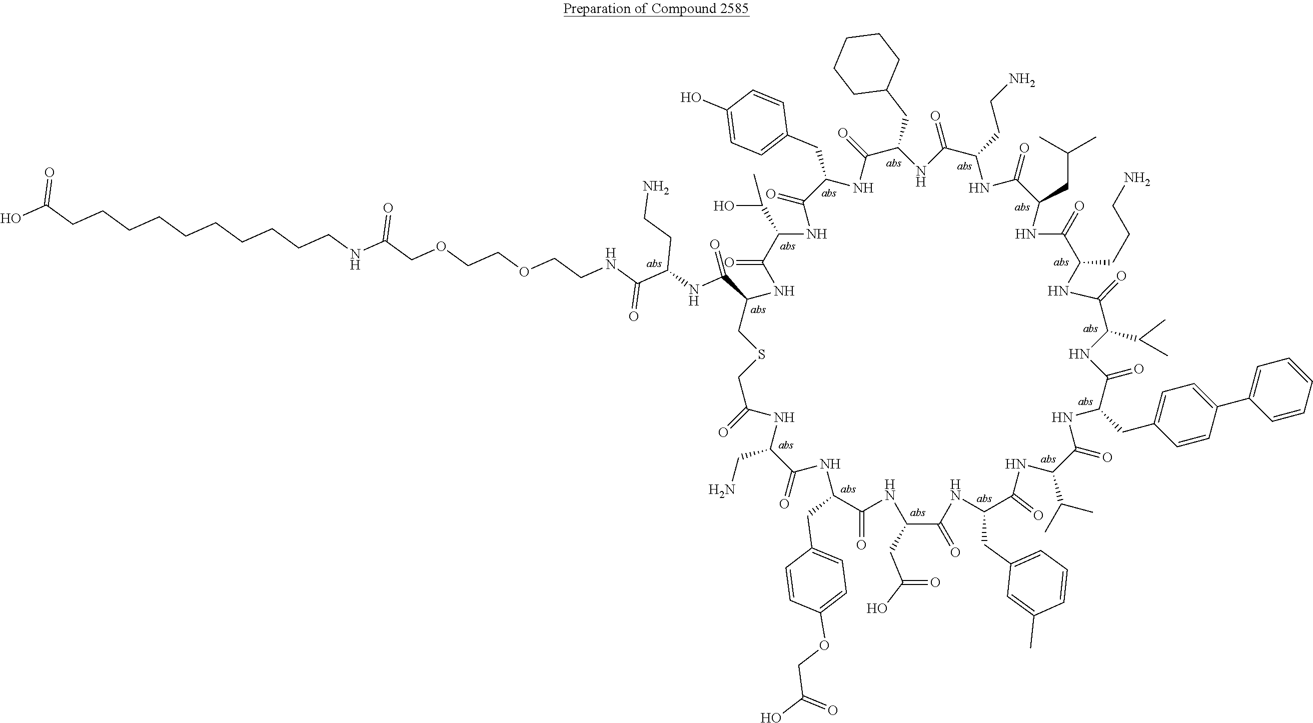 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2585 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 29.7 mg, and its estimated purity by LCMS analysis was 100%. Analysis condition A: Retention time=1.7 min; ESI-MS(+) m/z [M+3H]³⁺: 781.

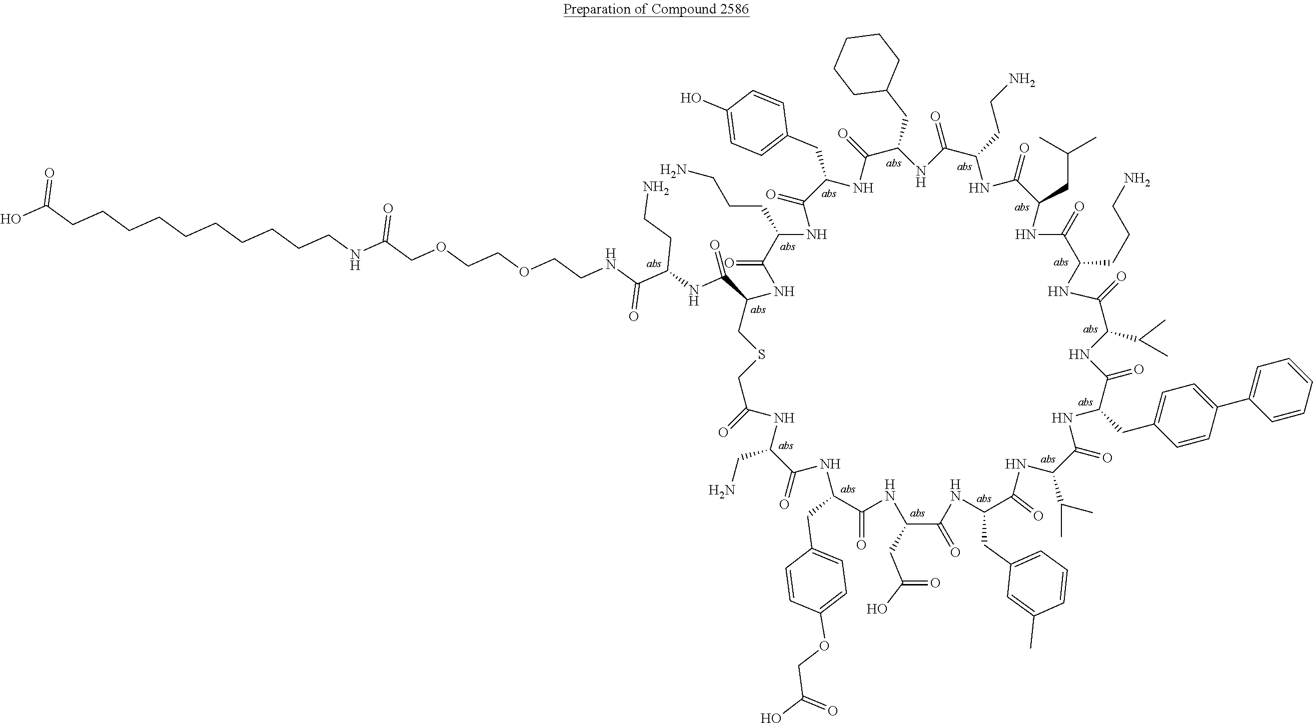 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](Cc3ccc(O)cc3)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2586 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.4 mg, and its estimated purity by LCMS analysis was 85.8%. Analysis condition B: Retention time=1.57 min; ESI-MS(+) m/z [M+2H]²⁺: 1177.1.

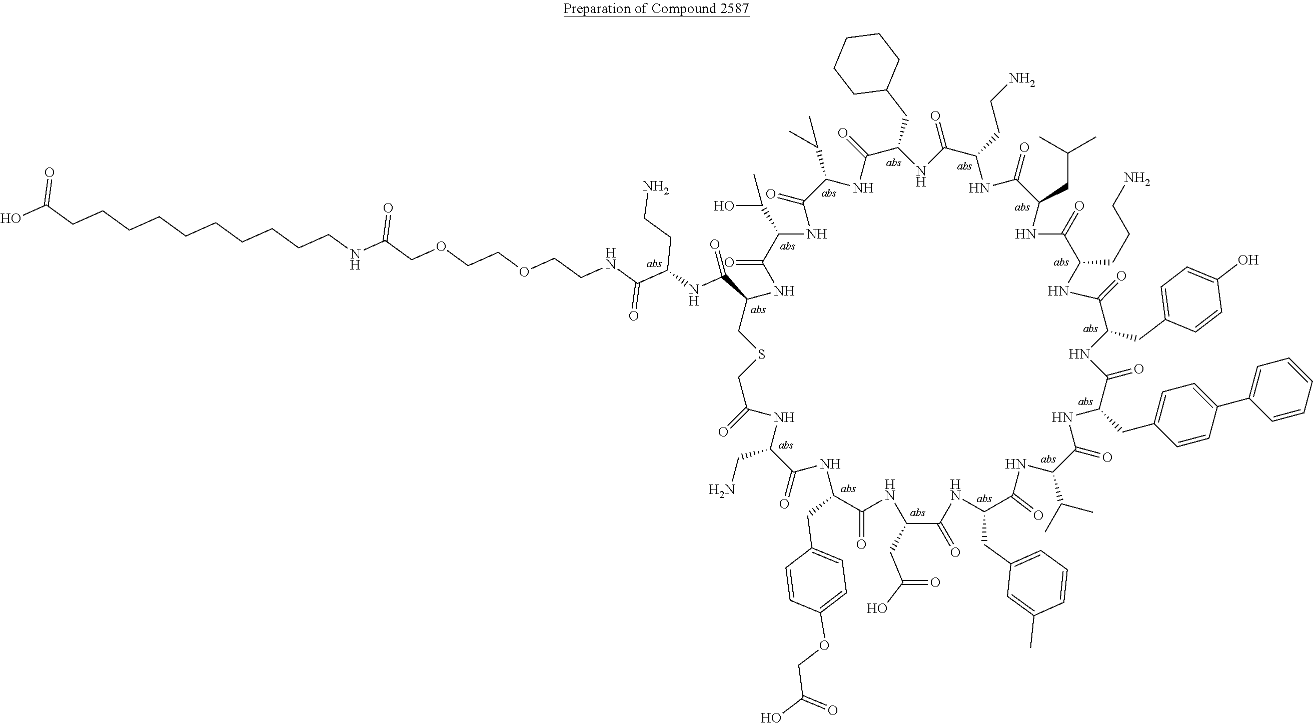 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2587 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 30.5 mg, and its estimated purity by LCMS analysis was 96.3%. Analysis condition B: Retention time=1.61 min; ESI-MS(+) m/z [M+3H]³⁺: 781.1.

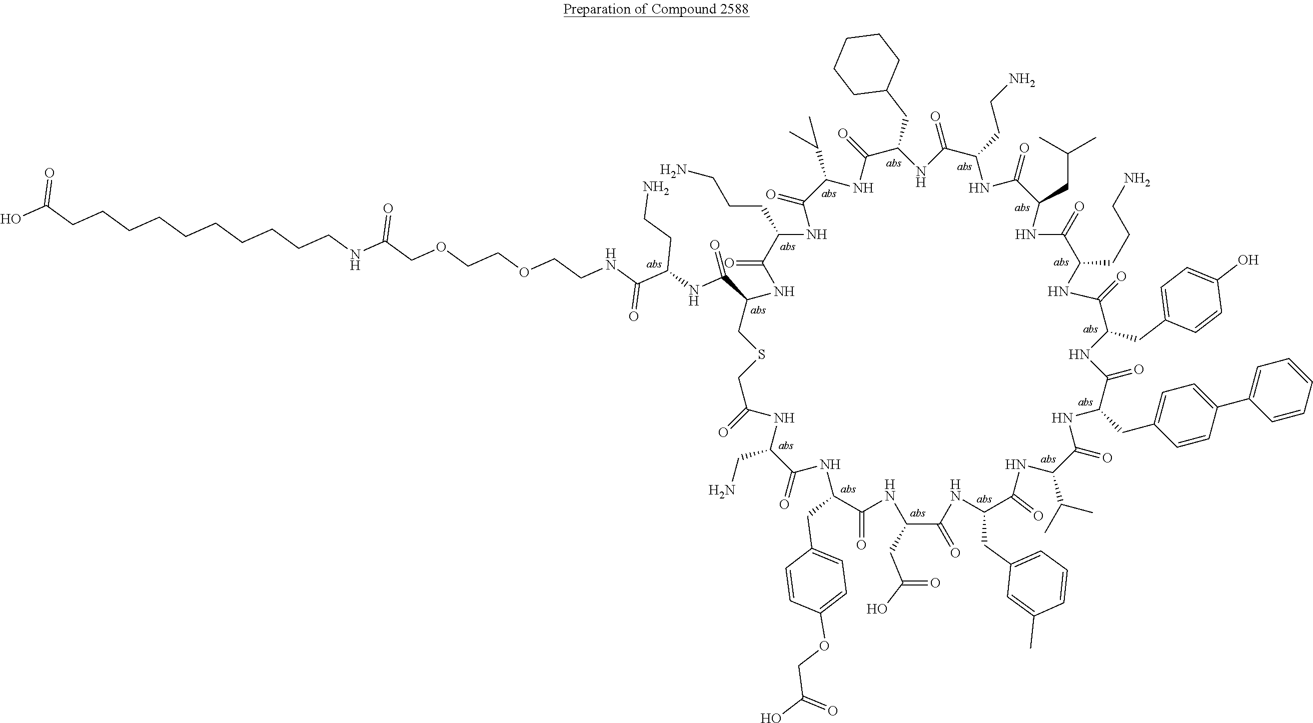 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2588 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-50% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 52.3 mg, and its estimated purity by LCMS analysis was 95.3%. Analysis condition A: Retention time=1.63 min; ESI-MS(+) m/z [M+3H]³⁺: 785.1.

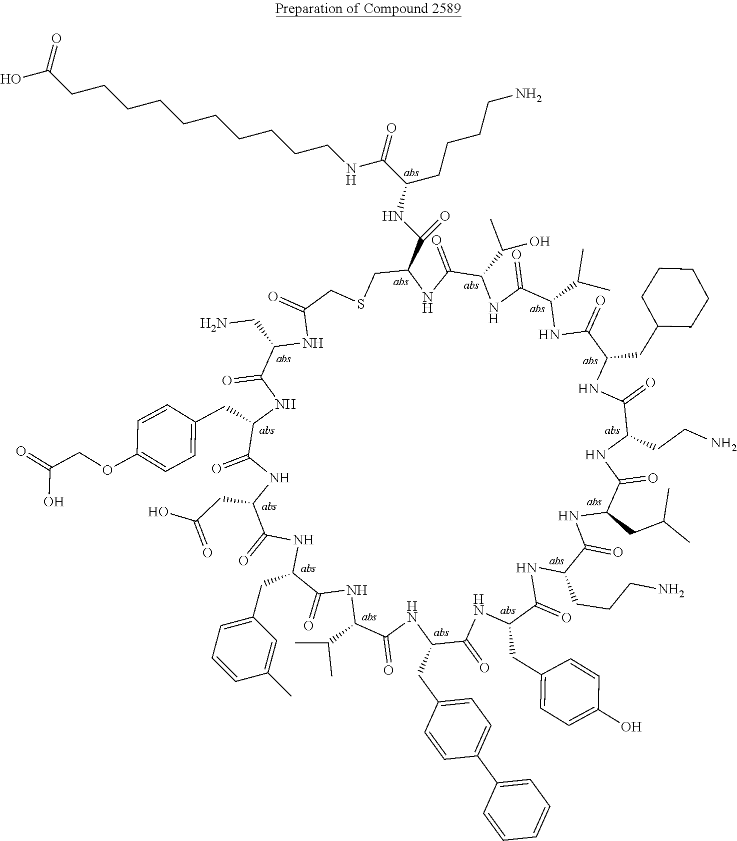 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2589 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-52% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 27.3 mg, and its estimated purity by LCMS analysis was 100%. Analysis condition A: Retention time=1.6 min; ESI-MS(+) m/z [M+2H]²⁺: 1111.8.

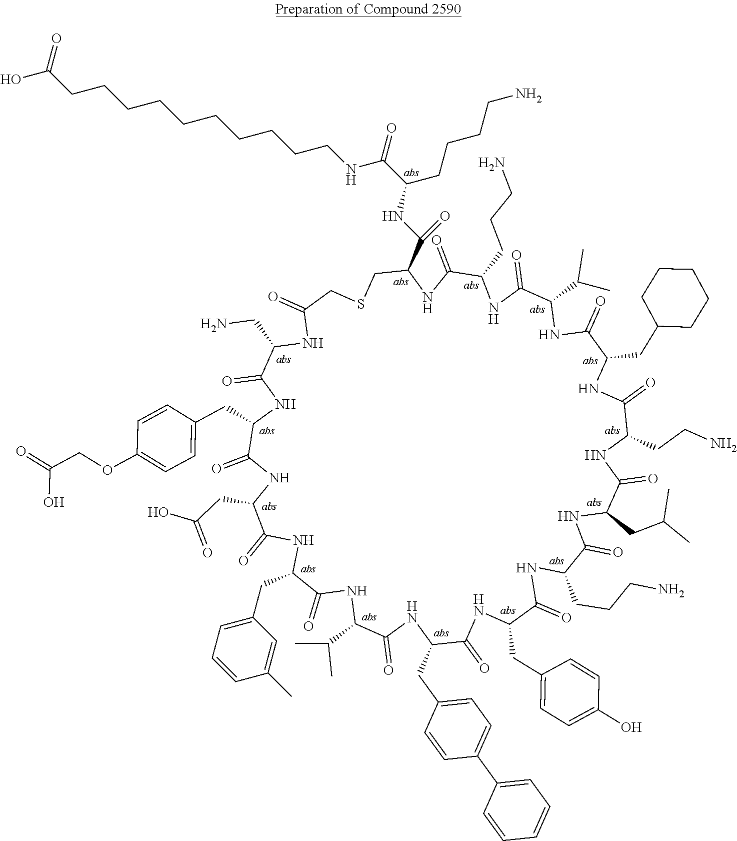 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2590 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 19.1 mg, and its estimated purity by LCMS analysis was 98.4%. Analysis condition B: Retention time=1.34 min; ESI-MS(+) m/z [M+3H]³⁺: 746.

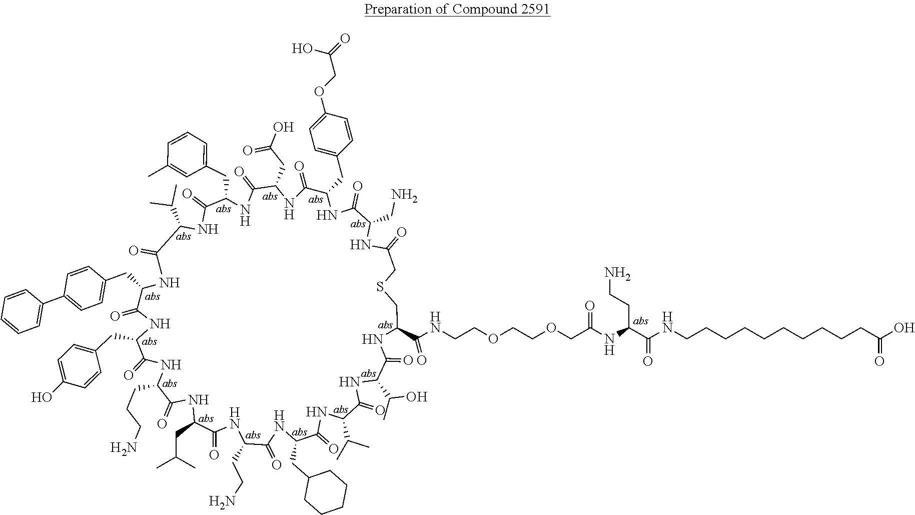 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2591 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-55% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 25.5 mg, and its estimated purity by LCMS analysis was 98.2%. Analysis condition B: Retention time=1.59 min; ESI-MS(+) m/z [M+2H]²⁺: 1170.5.

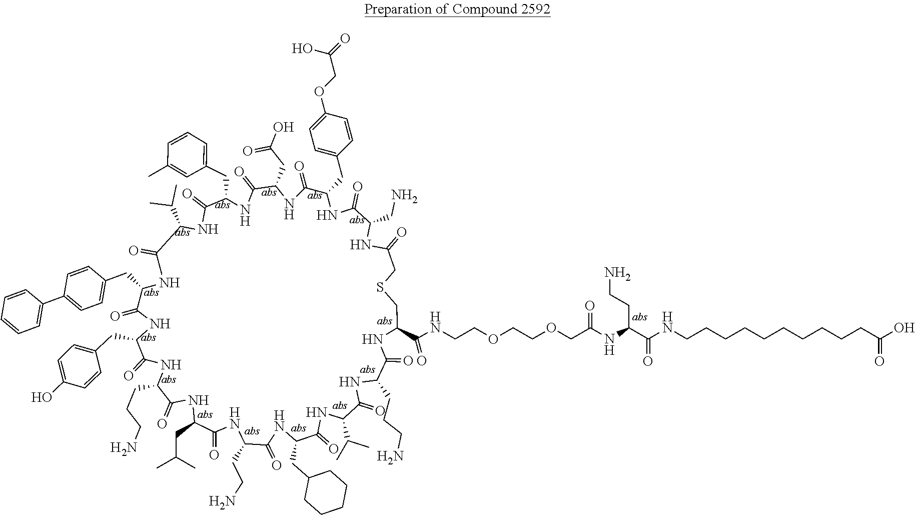 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2592 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.5 mg, and its estimated purity by LCMS analysis was 93.9%. Analysis condition A: Retention time=1.59 min; ESI-MS(+) m/z [M+3H]²⁺: 785.1.

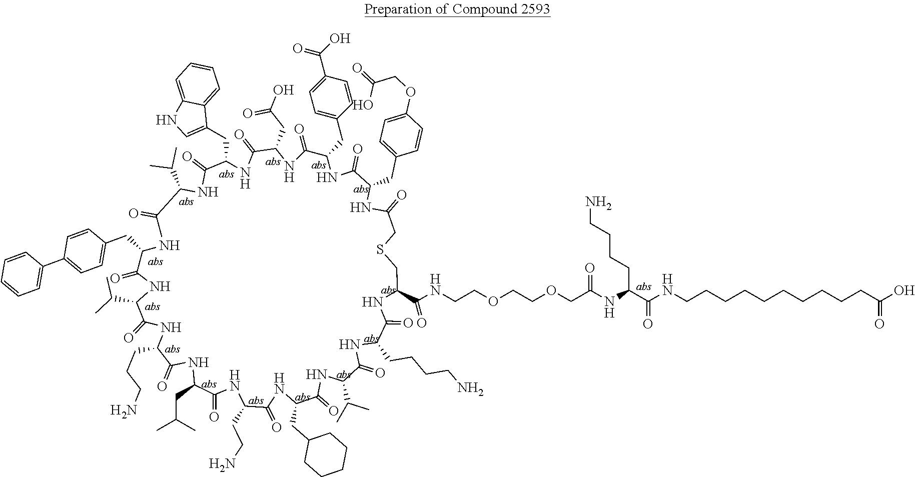 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@@H](CCCCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2593 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9 mg, and its estimated purity by LCMS analysis was 95%. Analysis condition B: Retention time=1.45 min; ESI-MS(+) m/z [M+3H]³⁺: 821.1.

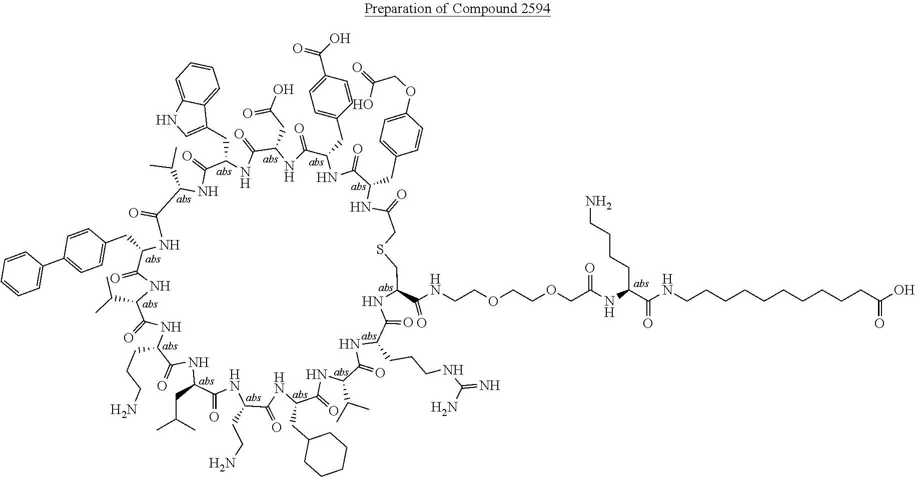 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@@H](CCCCN)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2594 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.7 mg, and its estimated purity by LCMS analysis was 92.6%. Analysis condition A: Retention time=1.66 min; ESI-MS(+) m/z [M+2H]²⁺: 1260.

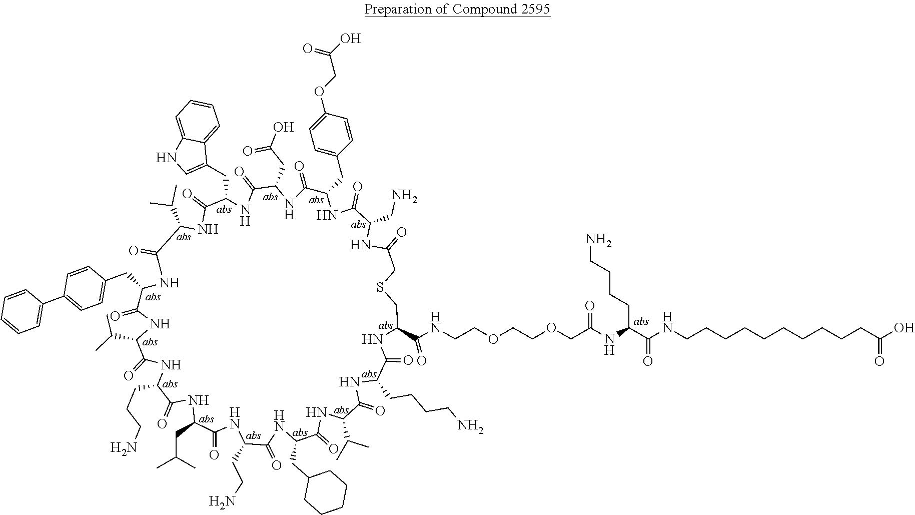 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2595 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 22 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.8 mg, and its estimated purity by LCMS analysis was 95.9%. Analysis condition B: Retention time=1.36 min; ESI-MS(+) m/z [M+3H]³⁺: 785.8.

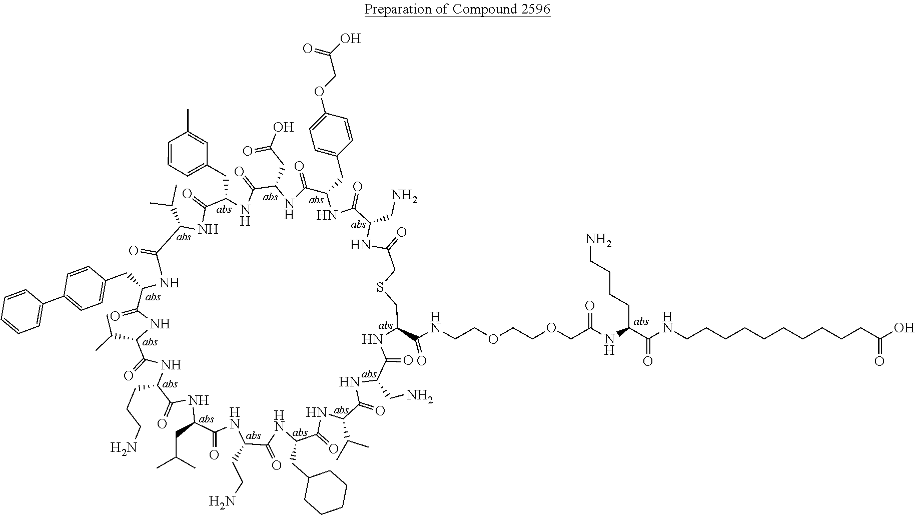 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2596 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 17.2 mg, and its estimated purity by LCMS analysis was 92.5%. Analysis condition A: Retention time=1.6 min; ESI-MS(+) m/z [M+2H]²⁺: 1144.9.

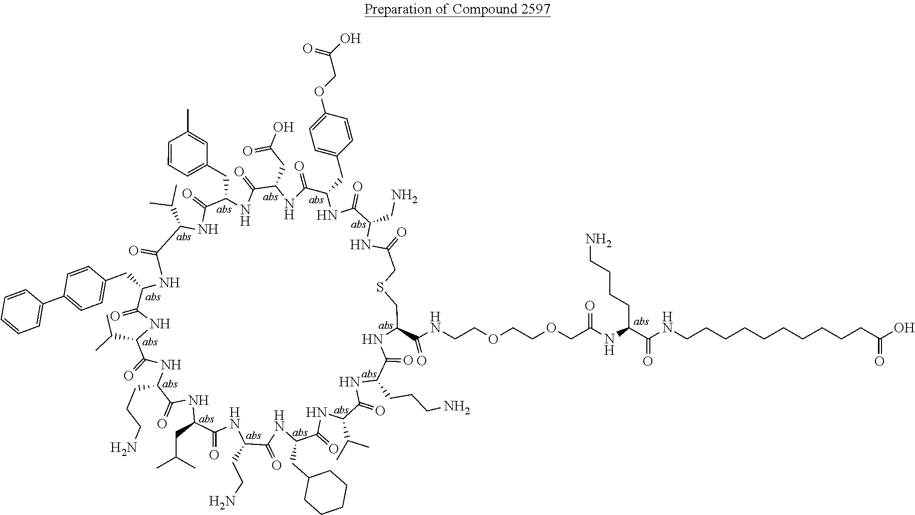 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2597 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.8 mg, and its estimated purity by LCMS analysis was 91.5%. Analysis condition B: Retention time=1.48 min; ESI-MS(+) m/z [M+2H]²⁺: 1158.8.

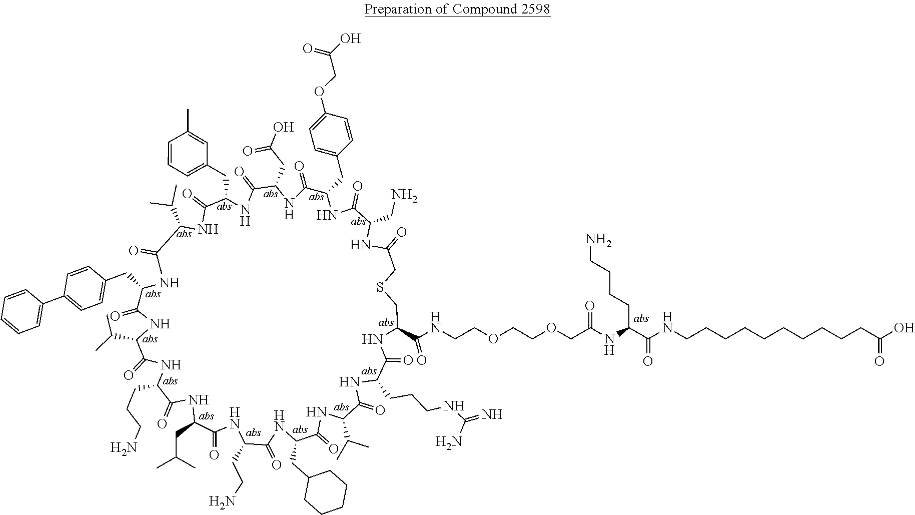 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2598 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.8 mg, and its estimated purity by LCMS analysis was 96.9%. Analysis condition B: Retention time=1.48 min; ESI-MS(+) m/z [M+2H]²⁺: 1180.2.

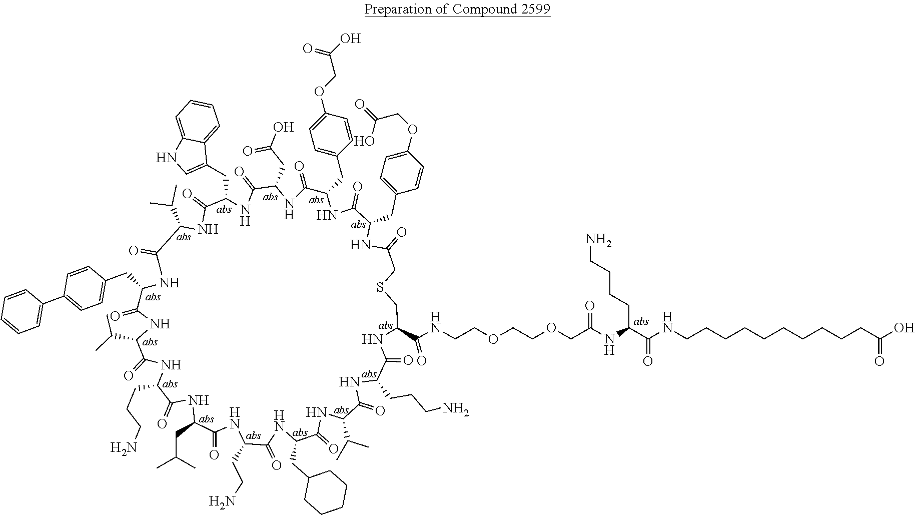 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2599 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 6.6 mg, and its estimated purity by LCMS analysis was 92.3%. Analysis condition A: Retention time=1.63 min; ESI-MS(+) m/z [M+2H]²⁺: 1239.2.

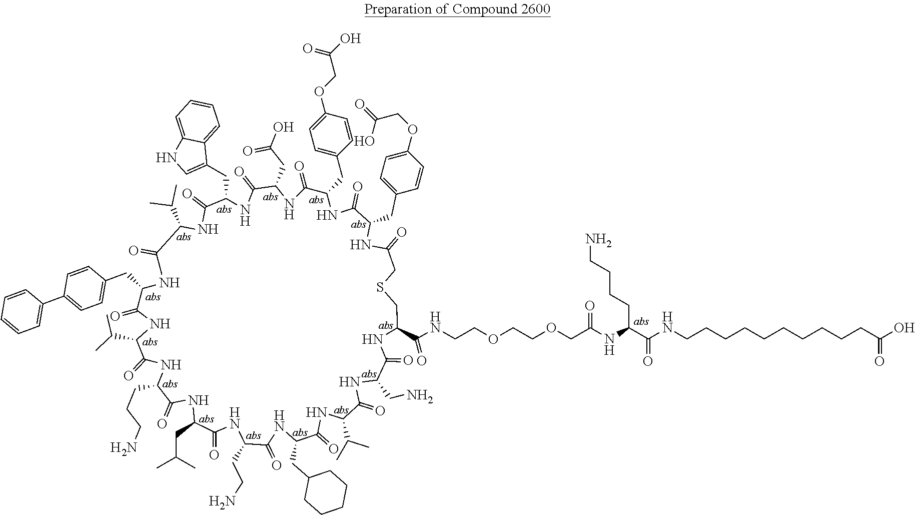 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2600 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 3.7 mg, and its estimated purity by LCMS analysis was 95.9%. Analysis condition A: Retention time=1.67 min; ESI-MS(+) m/z [M+2H]²⁺: 1225.2.

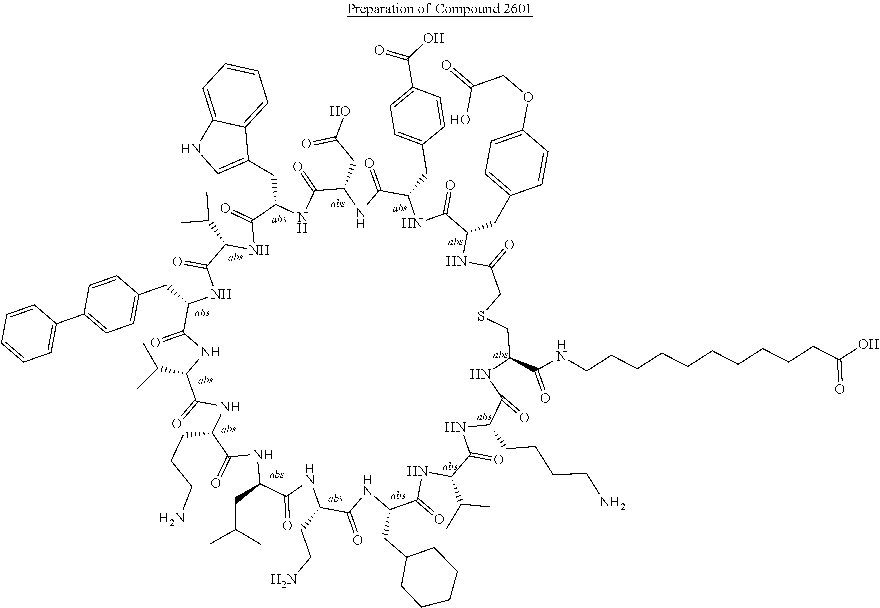 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2601 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 2-minute hold at centrifugal evaporation. The yield of the product was 2 mg, and its estimated purity by LCMS analysis was 92.2%. Analysis condition A: Retention time=1.69 min; ESI-MS(+) m/z [M+3H]³⁺: 730.

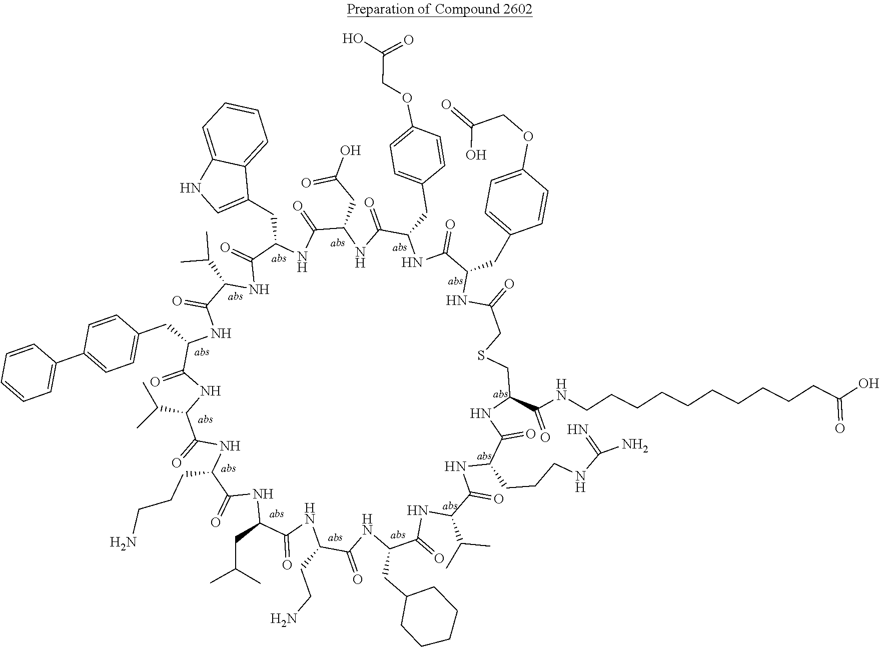 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2602 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 2-minute hold at by MS and UV signals. Fractions containing the desired product were combined and dried via centrifugal evaporation. The yield of the product was 3 mg, and its estimated purity by LCMS analysis was 100%. Analysis condition A: Retention time=1.68 min; ESI-MS(+) m/z [M+2H]²⁺: 1123.9.

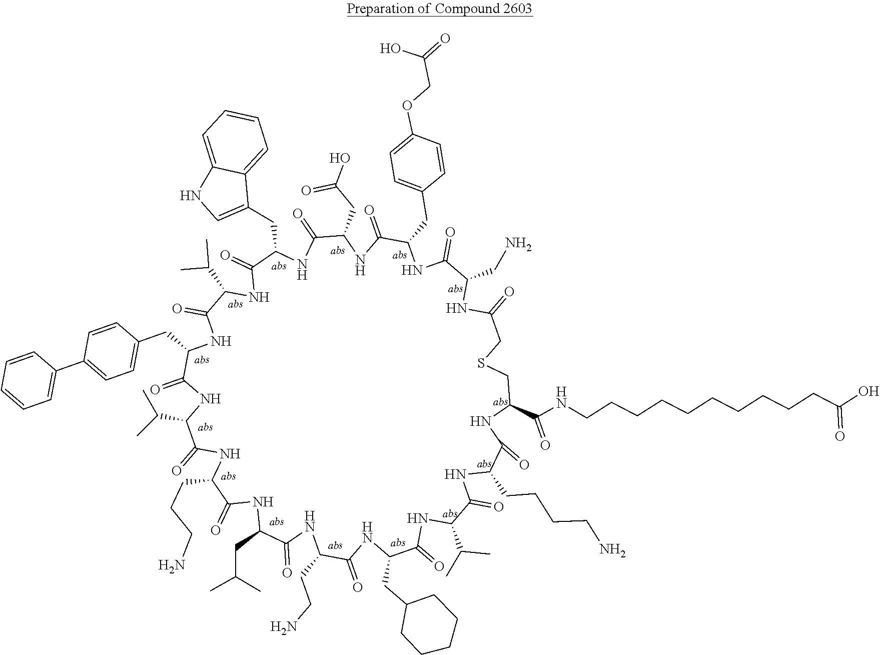 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2603 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 4.2 mg, and its estimated purity by LCMS analysis was 100%. Analysis condition A: Retention time=1.55 min; ESI-MS(+) m/z [M+2H]²⁺: 1042.1.

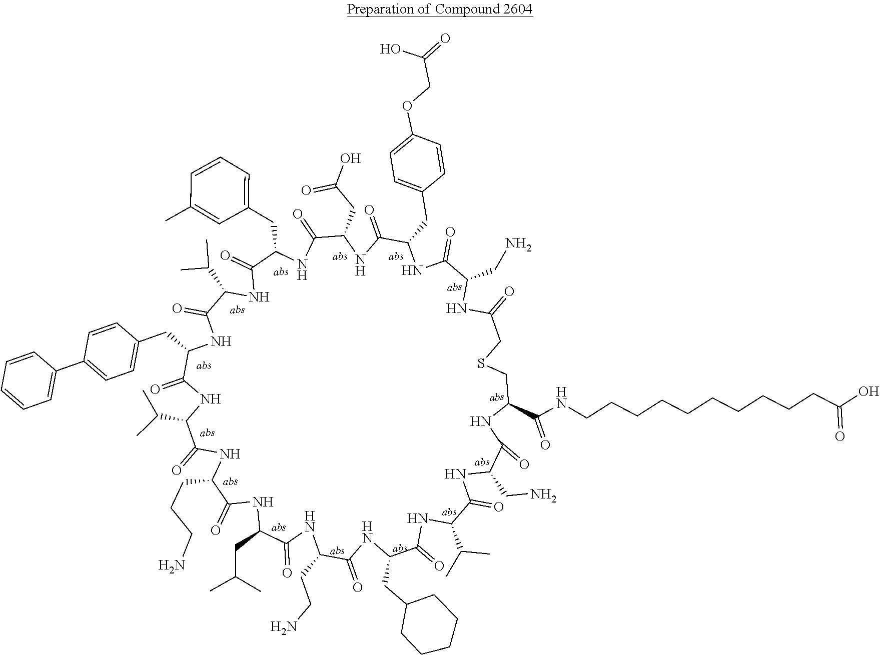 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2604 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 24% B, 24-64% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 12.8 mg, and its estimated purity by LCMS analysis was 93.2%. Analysis condition B: Retention time=1.66 min; ESI-MS(+) m/z [M+3H]³⁺: 673.

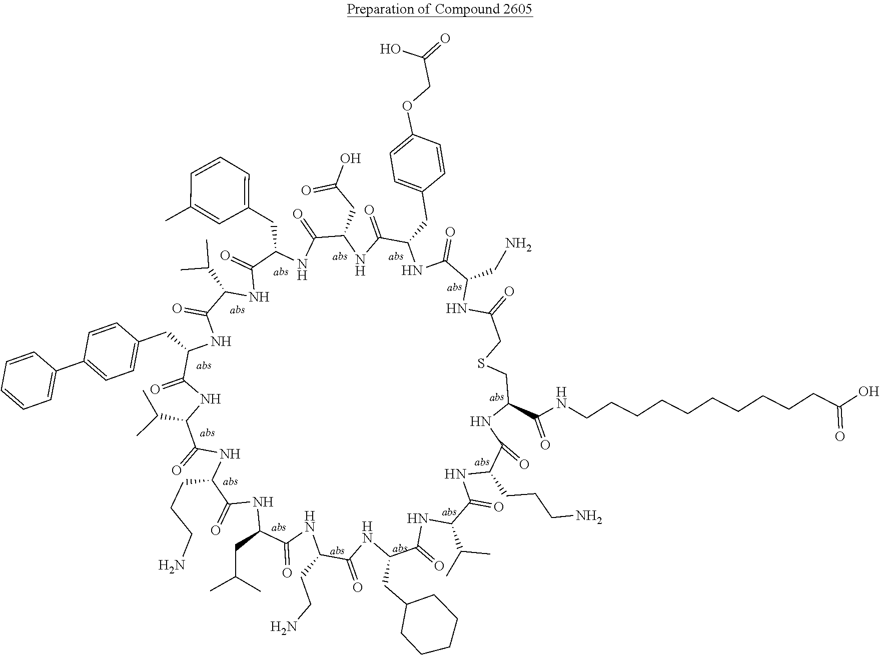 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2605 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-52% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 4.7 mg, and its estimated purity by LCMS analysis was 100%. Analysis condition A: Retention time=1.62 min; ESI-MS(+) m/z [M+3H]³⁺: 682.2.

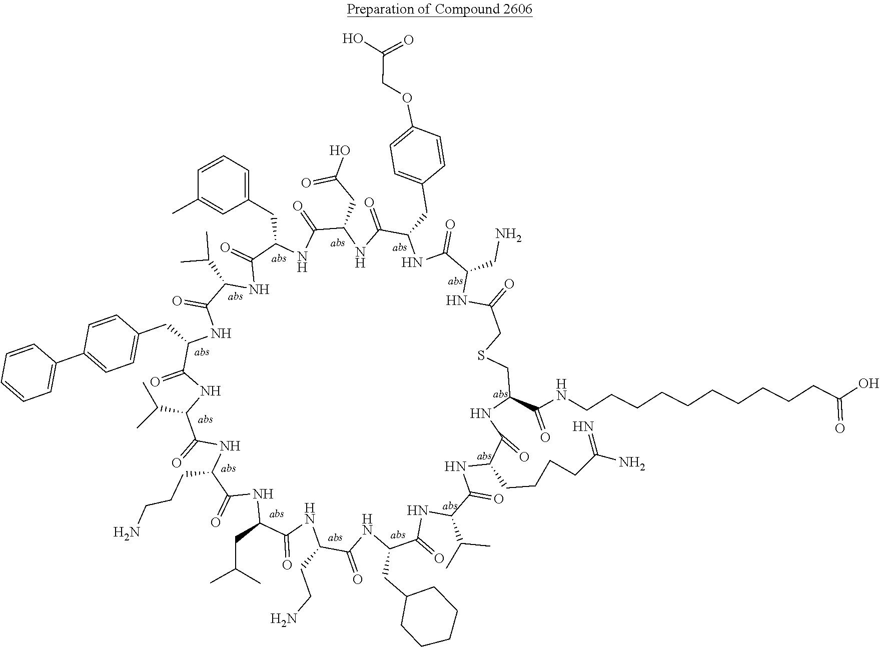 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCCCCCCCCCC(=O)O)NC(=O)[C@H](CCCCC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2606 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 7.9 mg, and its estimated purity by LCMS analysis was 100%. Analysis condition A: Retention time=1.59 min; ESI-MS(+) m/z [M+3H]³⁺: 696.1.

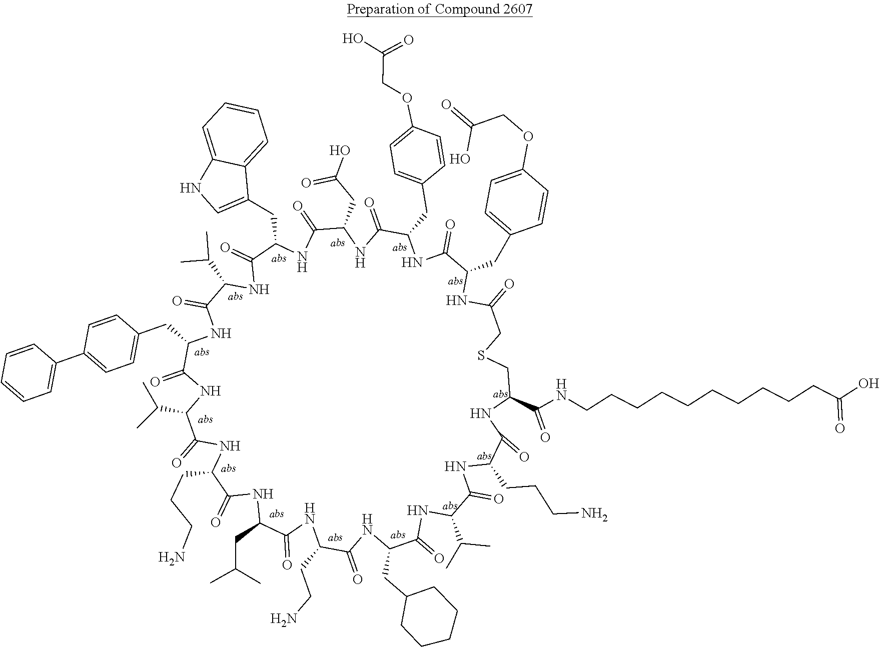 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2607 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.5 mg, and its estimated purity by LCMS analysis was 94.4%. Analysis condition A: Retention time=1.62 min; ESI-MS(+) m/z [M+2H]²⁺: 1102.8.

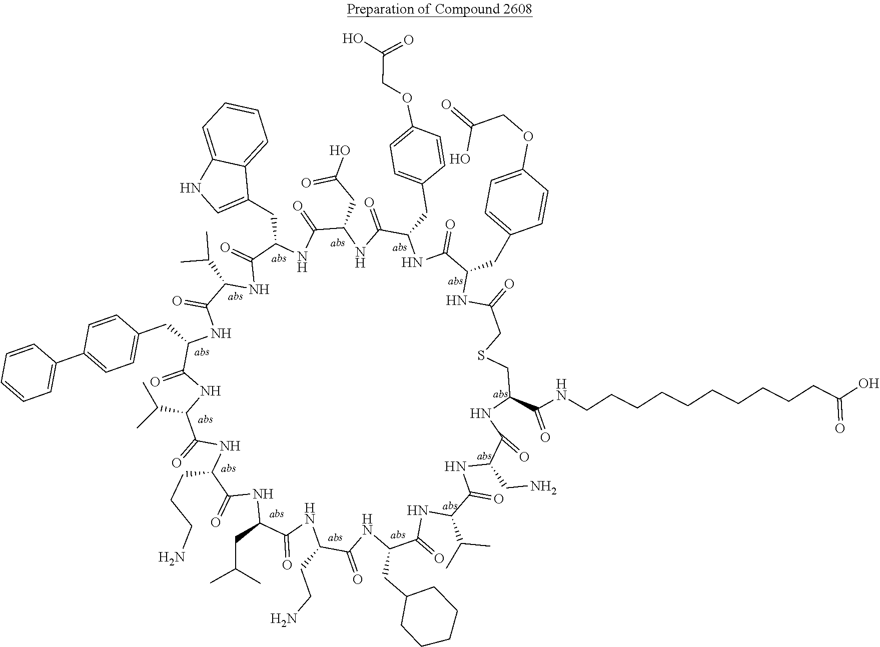 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2608 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.5 mg, and its estimated purity by LCMS analysis was 92.2%. Analysis condition A: Retention time=1.7, 1.77 min; ESI-MS(+) m/z [M+3H]³⁺: 725.85, 725.6.

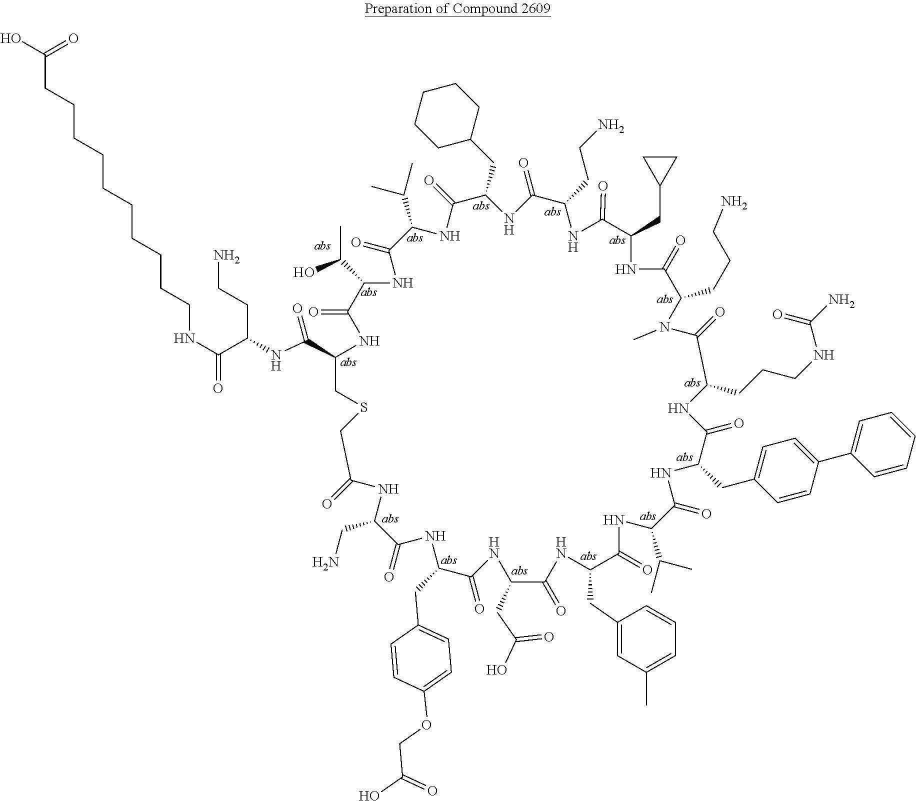 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@H](C)O)NC(=O)[C@H](C(C)C)NC(=O)[C@H](CC3CCCCC3)NC(=O)[C@H](CCN)NC(=O)[C@@H](CC3CC3)NC(=O)[C@H](CCCN)N(C)C(=O)[C@H](CCCNC(N)=O)NC(=O)[C@H](Cc3ccc(-c4ccccc4)cc3)NC(=O)[C@H](C(C)C)NC2=O)c1]

Compound 2609 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclopropyl)-OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-50% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.3 mg, and its estimated purity by LCMS analysis was 95.5%. Analysis condition A: Retention time=1.55 min; ESI-MS(+) m/z [M+3H]³⁺: 730.1.

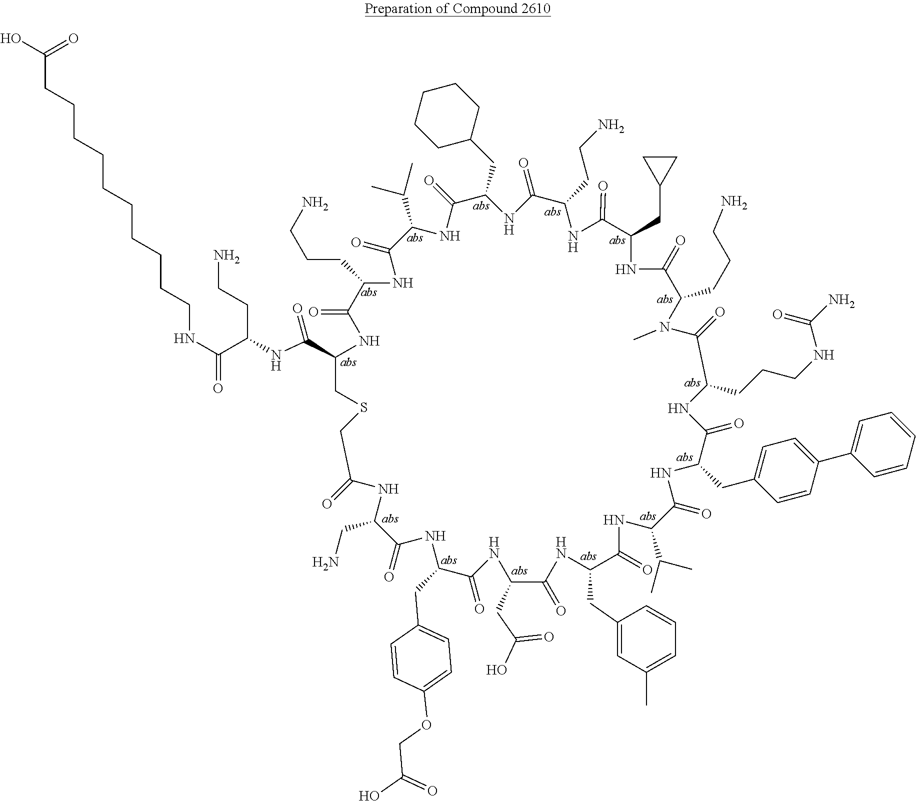 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC3CC3)NC(=O)[C@H](CCCN)N(C)C(=O)[C@H](CCCNC(N)=O)NC(=O)[C@H](Cc3ccc(-c4ccccc4)cc3)NC(=O)[C@H](C(C)C)NC2=O)c1]

Compound 2610 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclopropyl)-OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-47% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.8 mg, and its estimated purity by LCMS analysis was 97.2%. Analysis condition A: Retention time=1.41 min; ESI-MS(+) m/z [M+2H]²⁺: 1100.2.

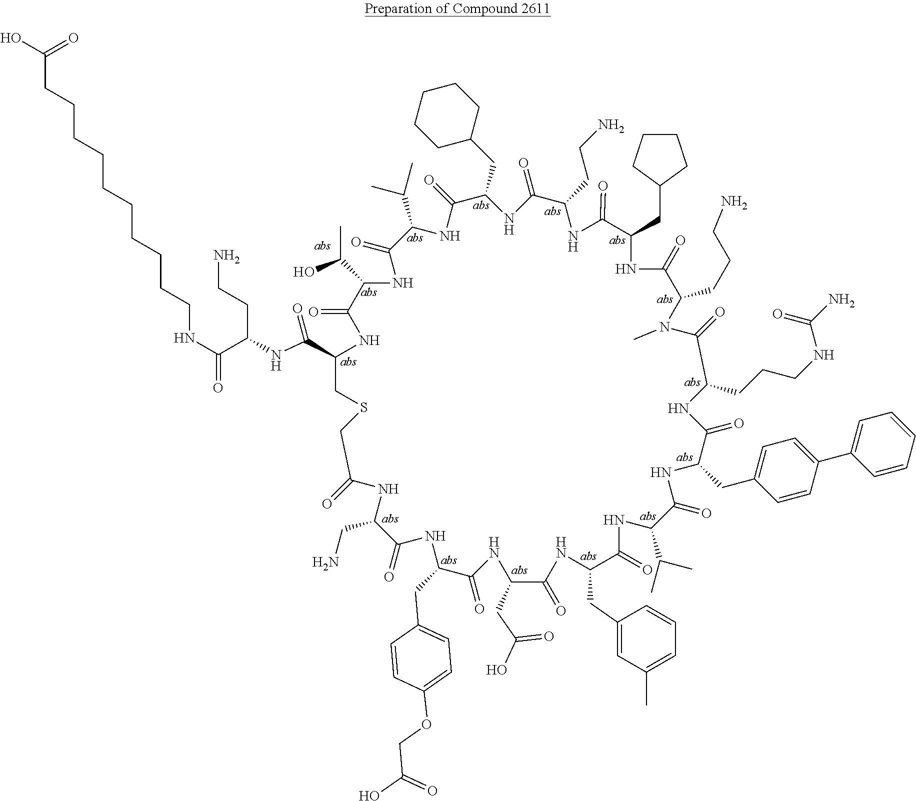 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@H](C)O)NC(=O)[C@H](C(C)C)NC(=O)[C@H](CC3CCCCC3)NC(=O)[C@H](CCN)NC(=O)[C@@H](CC3CCCC3)NC(=O)[C@H](CCCN)N(C)C(=O)[C@H](CCCNC(N)=O)NC(=O)[C@H](Cc3ccc(-c4ccccc4)cc3)NC(=O)[C@H](C(C)C)NC2=O)c1]

Compound 2611 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclopentyl)-OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.3 mg, and its estimated purity by LCMS analysis was 94.7%. Analysis condition A: Retention time=1.58 min; ESI-MS(+) m/z [M+2H]²⁺: 739.

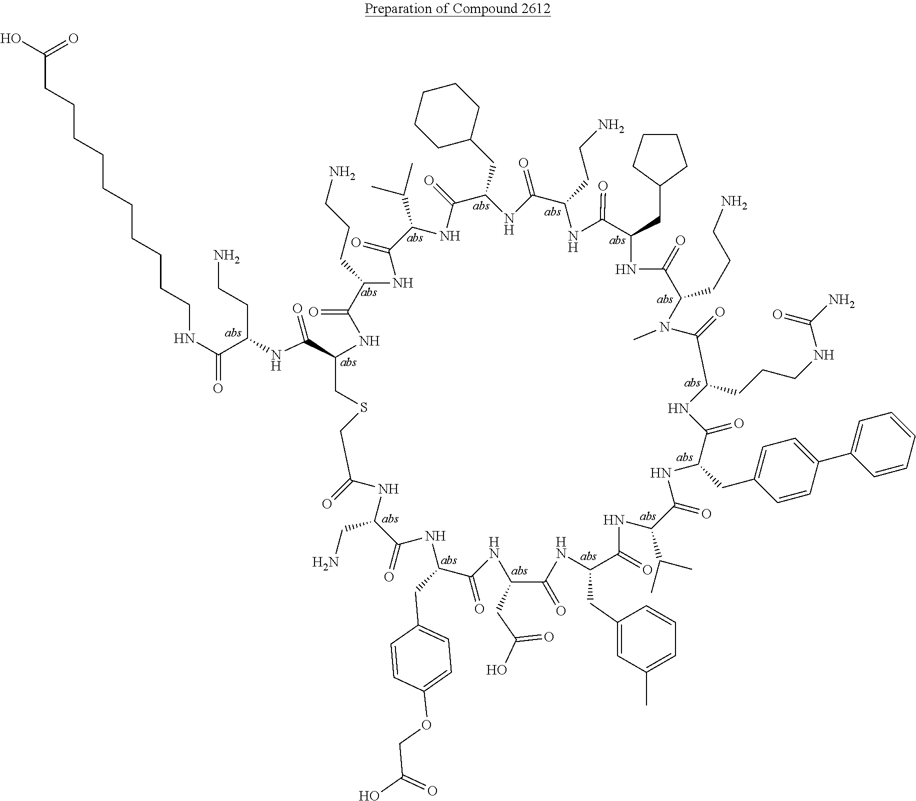 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC3CCCC3)NC(=O)[C@H](CCCN)N(C)C(=O)[C@H](CCCNC(N)=O)NC(=O)[C@H](Cc3ccc(-c4ccccc4)cc3)NC(=O)[C@H](C(C)C)NC2=O)c1]

Compound 2612 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclopentyl)-OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.3 mg, and its estimated purity by LCMS analysis was 92.9%. Analysis condition B: Retention time=1.54 min; ESI-MS(+) m/z [M+2H]²⁺: 1115.

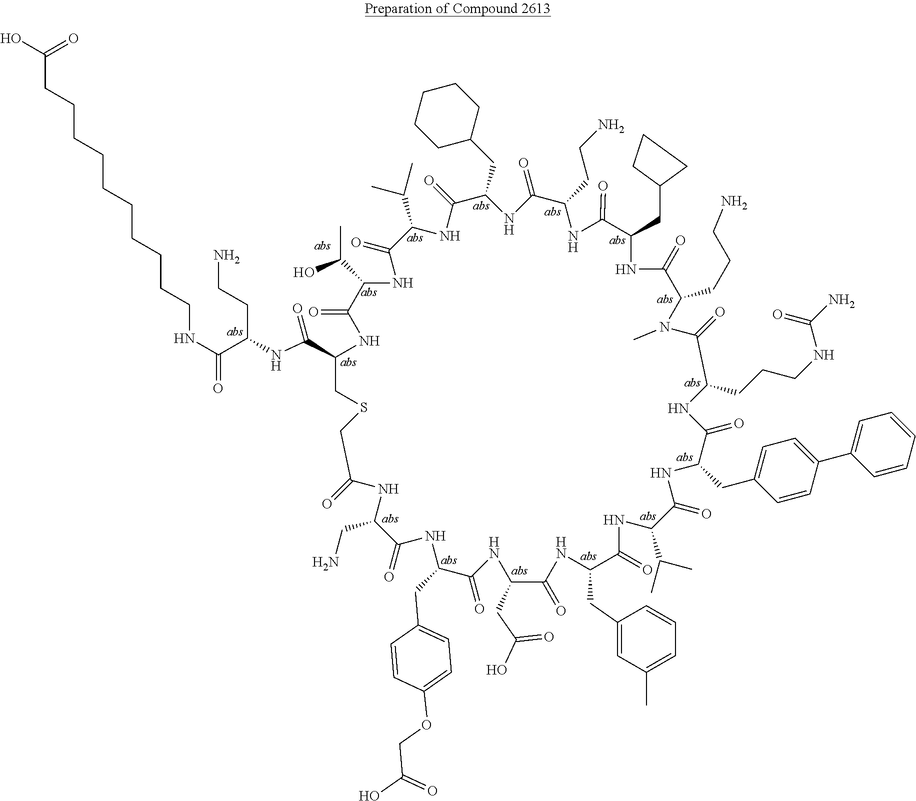 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@H](C)O)NC(=O)[C@H](C(C)C)NC(=O)[C@H](CC3CCCCC3)NC(=O)[C@H](CCN)NC(=O)[C@@H](CC3CCC3)NC(=O)[C@H](CCCN)N(C)C(=O)[C@H](CCCNC(N)=O)NC(=O)[C@H](Cc3ccc(-c4ccccc4)cc3)NC(=O)[C@H](C(C)C)NC2=O)c1]

Compound 2613 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclobutyl)-OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.2 mg, and its estimated purity by LCMS analysis was 100%. Analysis condition A: Retention time=1.63 min; ESI-MS(+) m/z [M+2H]²⁺: 1100.8.

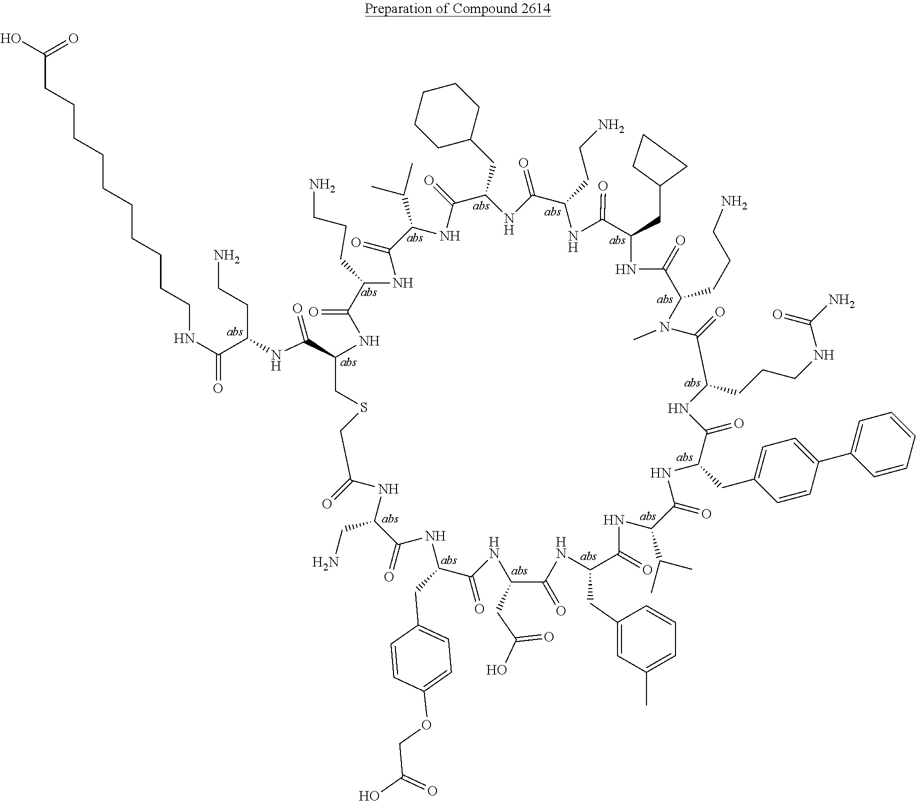 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC3CCC3)NC(=O)[C@H](CCCN)N(C)C(=O)[C@H](CCCNC(N)=O)NC(=O)[C@H](Cc3ccc(-c4ccccc4)cc3)NC(=O)[C@H](C(C)C)NC2=O)c1]

Compound 2614 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-D-Ala (cyclobutyl)-OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 3-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.5 mg, and its estimated purity by LCMS analysis was 100%. Analysis condition A: Retention time=1.59 min; ESI-MS(+) m/z [M+2H]²⁺: 1107.3.

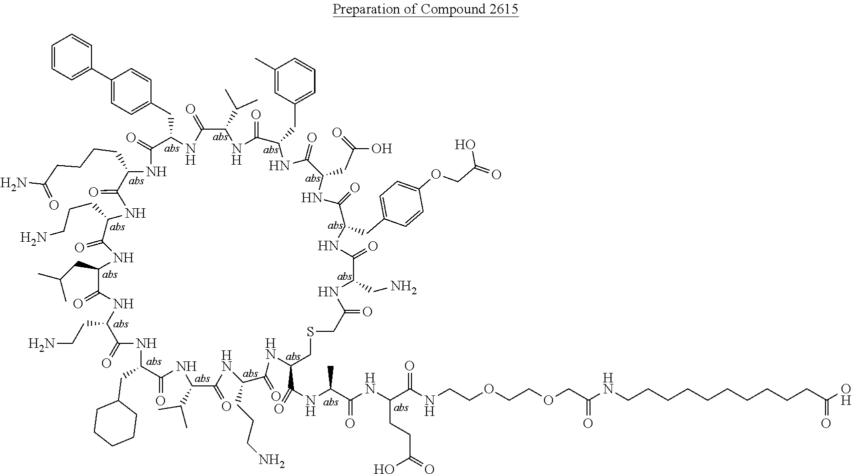 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)NC(CCC(=O)O)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCCC(N)=O)NC(=O)[C@H](Cc3ccc(-c4ccccc4)cc3)NC(=O)[C@H](C(C)C)NC2=O)c1]

Compound 2615 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 9.4 mg, and its estimated purity by LCMS analysis was 100%. Analysis condition A: Retention time=1.62 min; ESI-MS(+) m/z [M+2H]²⁺: 1224.1.

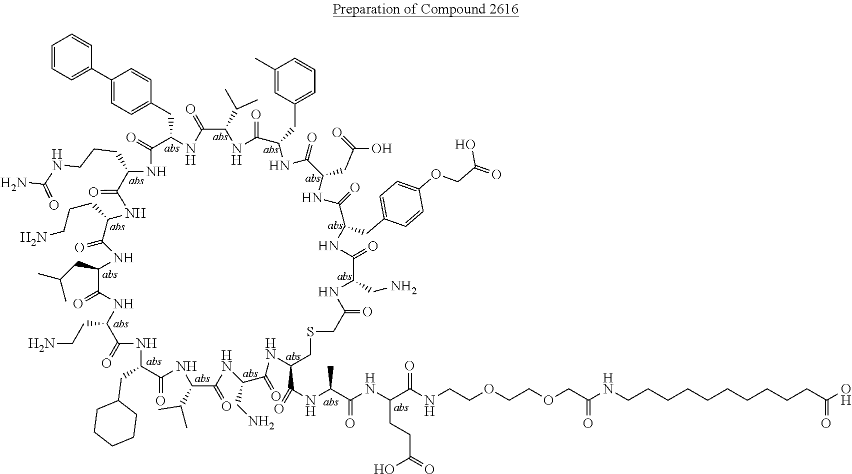 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)NC(CCC(=O)O)C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2616 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 21.5 mg, and its estimated purity by LCMS analysis was 95.9%. Analysis condition B: Retention time=1.58 min; ESI-MS(+) m/z [M+2H]²⁺: 1210.3.

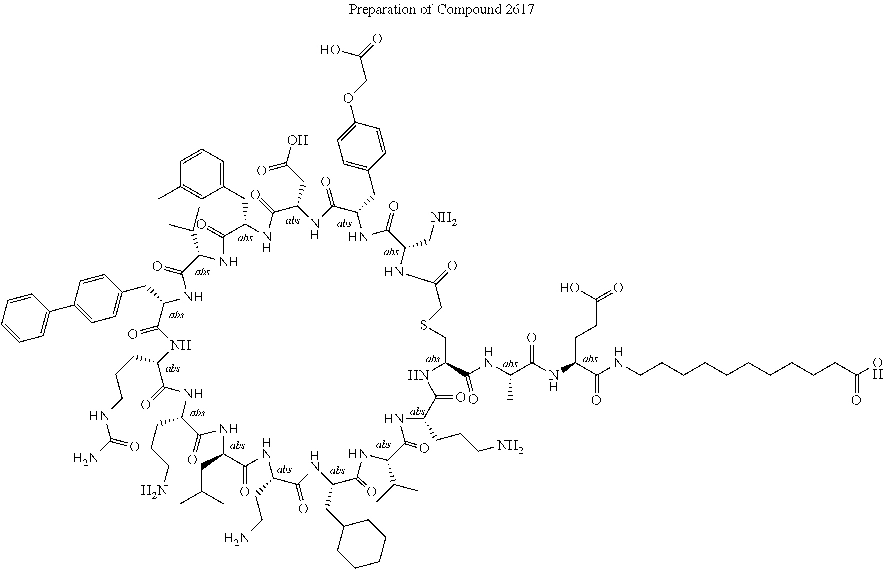 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2617 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-46% B over 25 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.2 mg, and its estimated purity by LCMS analysis was 93.2%. Analysis condition A: Retention time=1.53 min; ESI-MS(+) m/z [M+3H]³⁺: 768.1.

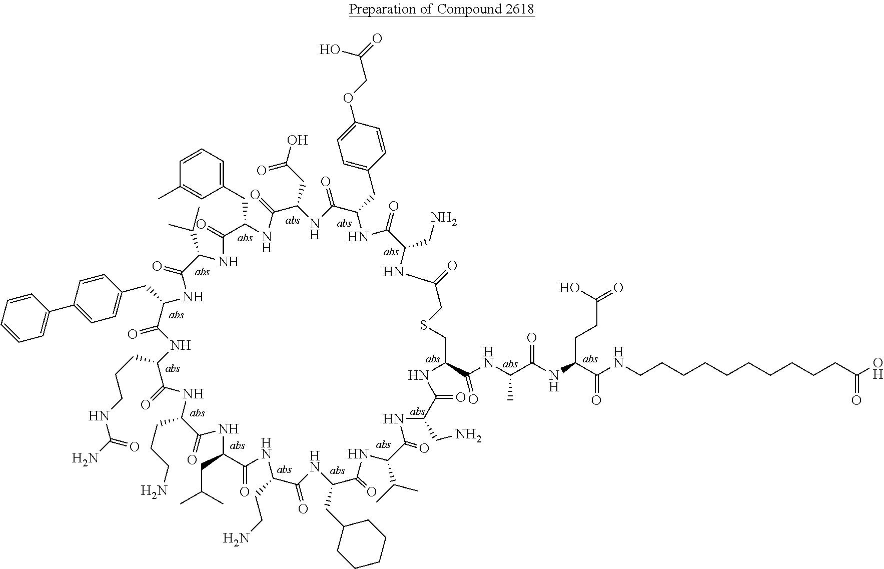 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2618 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-50% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 31.8 mg, and its estimated purity by LCMS analysis was 96.4%. Analysis condition B: Retention time=1.45 min; ESI-MS(+) m/z [M+2H]²⁺: 758.9.

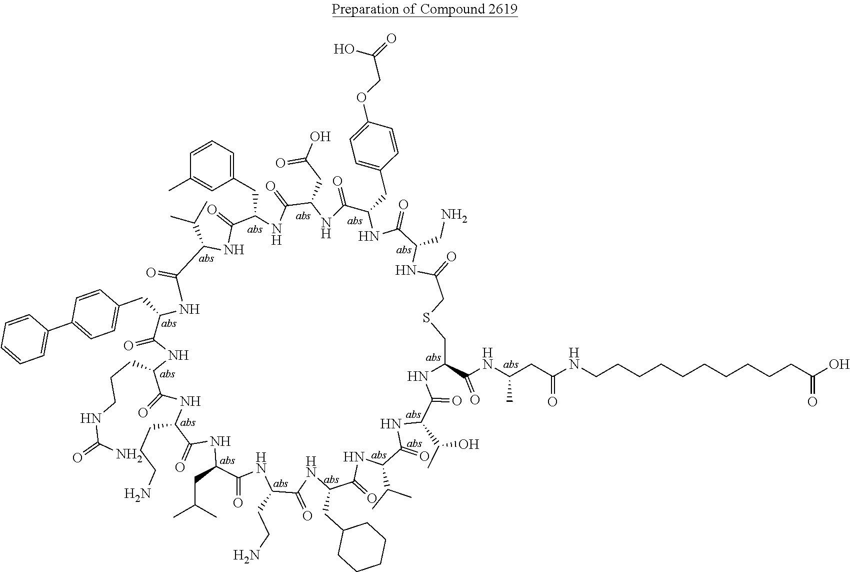 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)CC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2619 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 24% B, 24-64% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 6.7 mg, and its estimated purity by LCMS analysis was 95.7%. Analysis condition B: Retention time=1.64 min; ESI-MS(+) m/z [M+2H]²⁺: 1087.9.

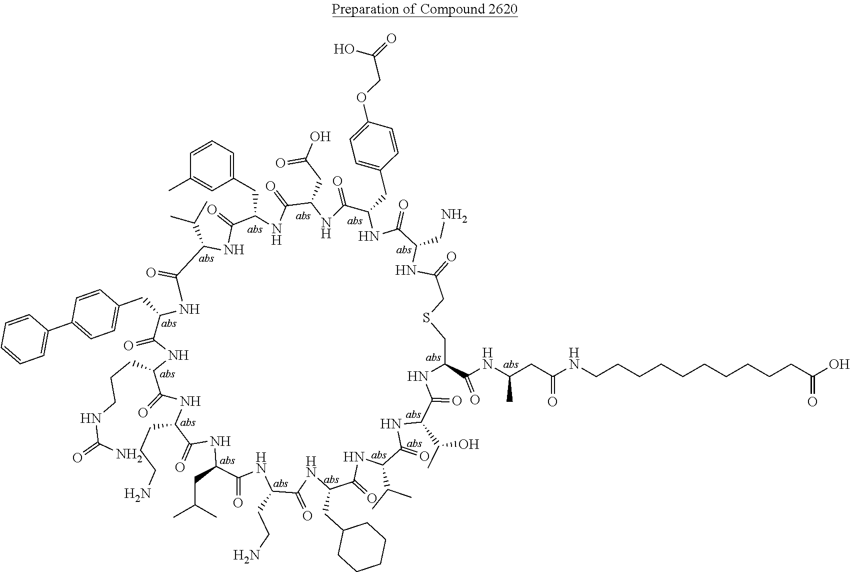 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@H](C)CC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2620 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 43.8 mg, and its estimated purity by LCMS analysis was 94.5%. Analysis condition B: Retention time=1.61 min; ESI-MS(+) m/z [M+3H]³⁺: 726.1.

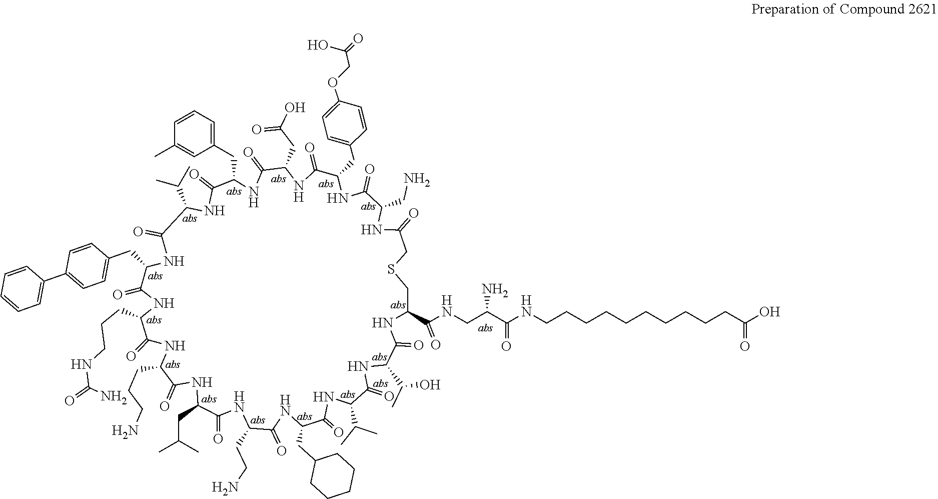 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NC[C@H](N)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2621 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.6 mg, and its estimated purity by LCMS analysis was 98.6%. Analysis condition A: Retention time=1.71 min; ESI-MS(+) m/z [M+2H]²⁺: 1088.1.

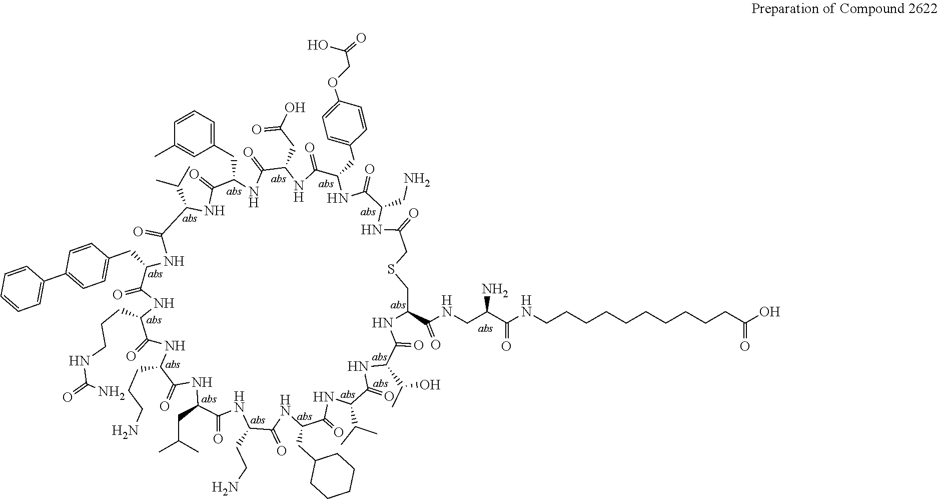 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NC[C@@H](N)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2622 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.1 mg, and its estimated purity by LCMS analysis was 97.4%. Analysis condition B: Retention time=1.57 min; ESI-MS(+) m/z [M+2H]²⁺: 1088.1.

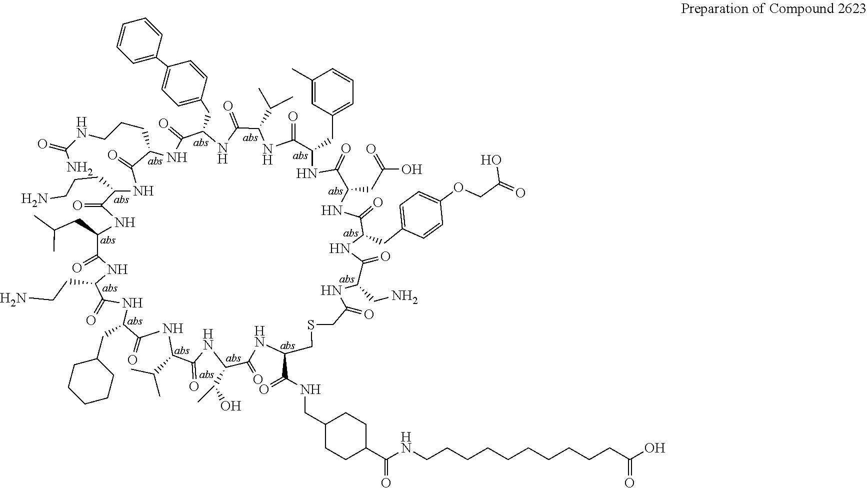 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC3CCC(C(=O)NCCCCCCCCCCC(=O)O)CC3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2623 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 24% B, 24-64% B over 20 minutes, then a 2-minute hold at 100% B; Flow Rate: 35 mL/min; Column Temperature: 25 C. Fraction collection was triggered by MS and UV signals. Fractions containing the desired product were combined and dried via centrifugal evaporation. The yield of the product was 29.8 mg, and its estimated purity by LCMS analysis was 93.1%. Analysis condition A: Retention time=1.7 min; ESI-MS(+) m/z [M+3H]³⁺: 743.

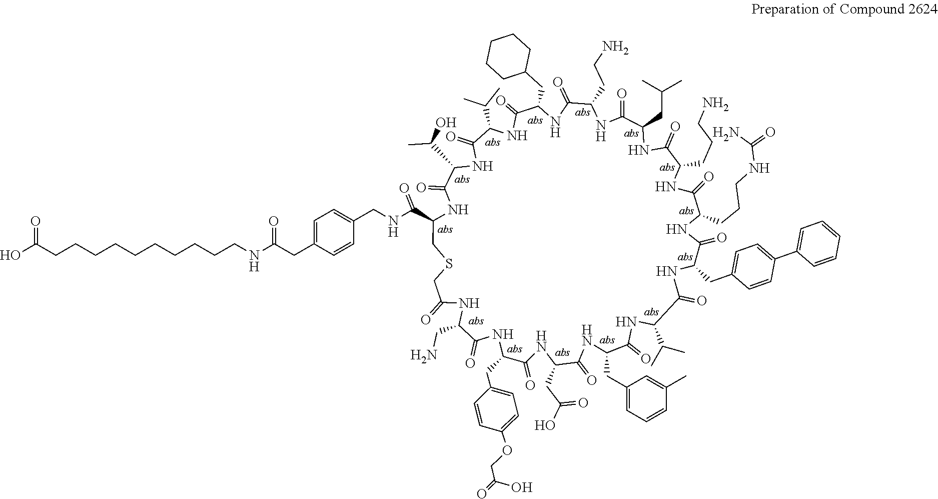 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCc3ccc(CC(=O)NCCCCCCCCCCC(=O)O)cc3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2624 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.7 mg, and its estimated purity by LCMS analysis was 100%. Analysis condition A: Retention time=1.7 min; ESI-MS(+) m/z [M+3H]³⁺: 745.7.

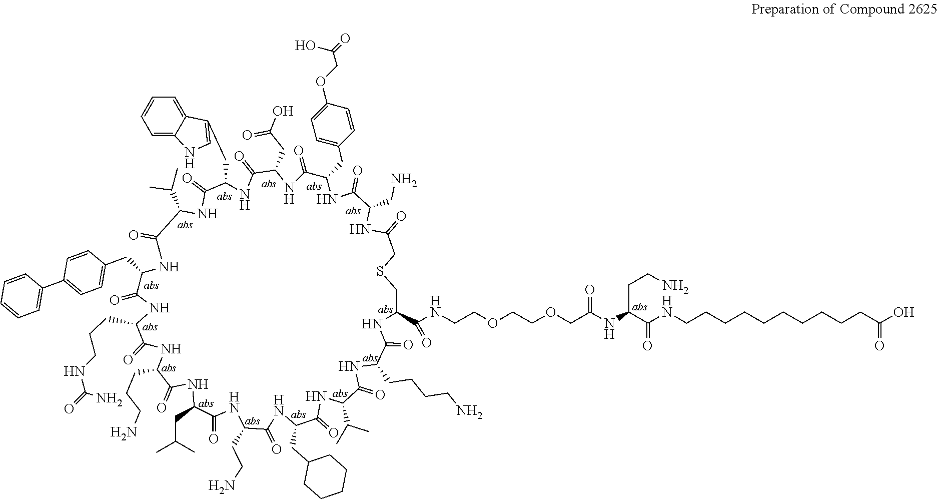 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2625 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.5 mg, and its estimated purity by LCMS analysis was 93.6%. Analysis condition B: Retention time=2.1 min; ESI-MS(+) m/z [M+3H]³⁺: 796.1.

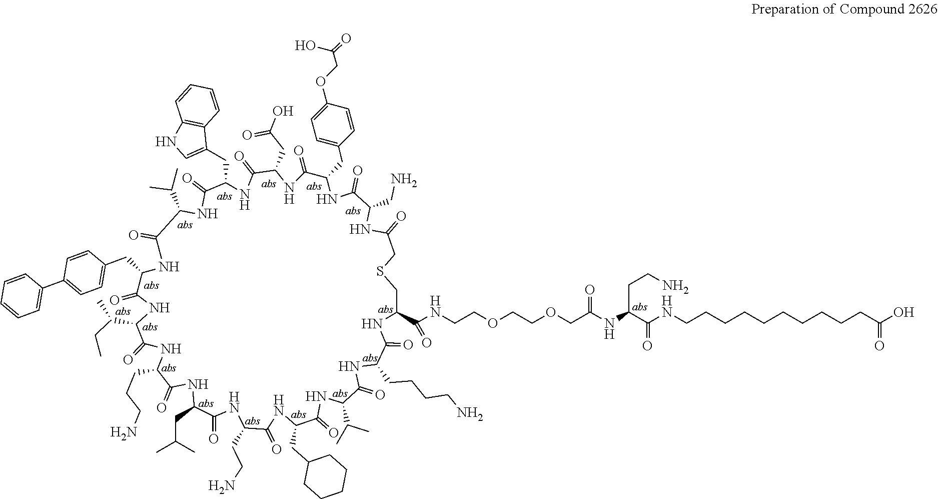 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2626 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8 mg, and its estimated purity by LCMS analysis was 95.8%. Analysis condition A: Retention time=1.42 min; ESI-MS(+) m/z [M+2H]²⁺: 1171.6.

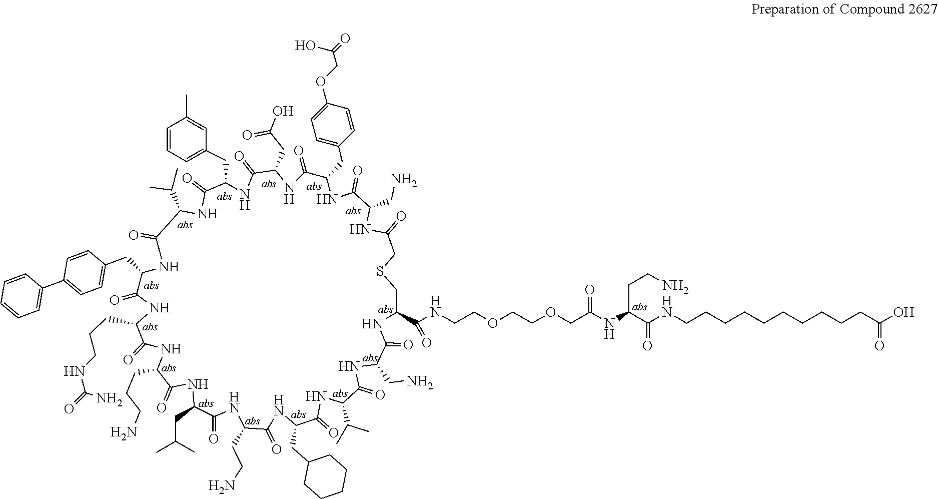 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2627 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 7 mg, and its estimated purity by LCMS analysis was 100%. Analysis condition A: Retention time=1.57 min; ESI-MS(+) m/z [M+2H]²⁺: 1159.8.

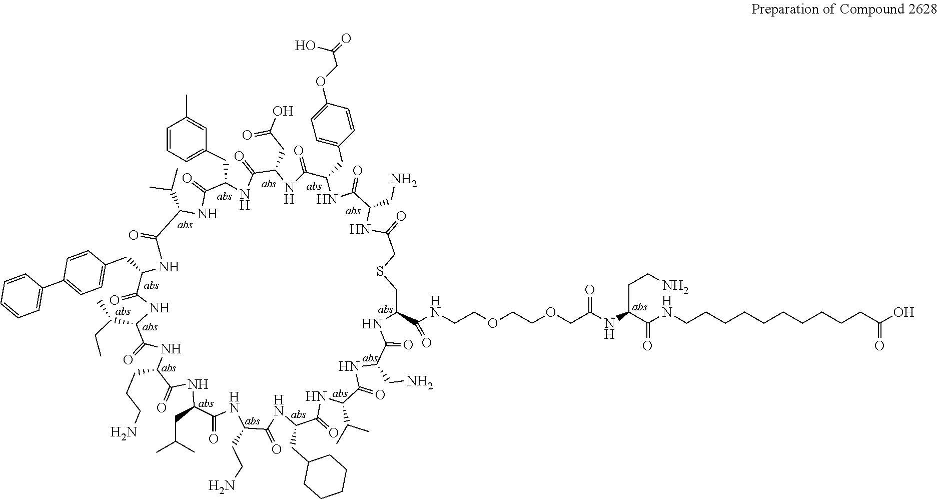 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2628 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9 mg, and its estimated purity by LCMS analysis was 95.8%. Analysis condition A: Retention time=1.58, 1.7 min; ESI-MS(+) m/z [M+3H]³⁺: 759.31, 759.22.

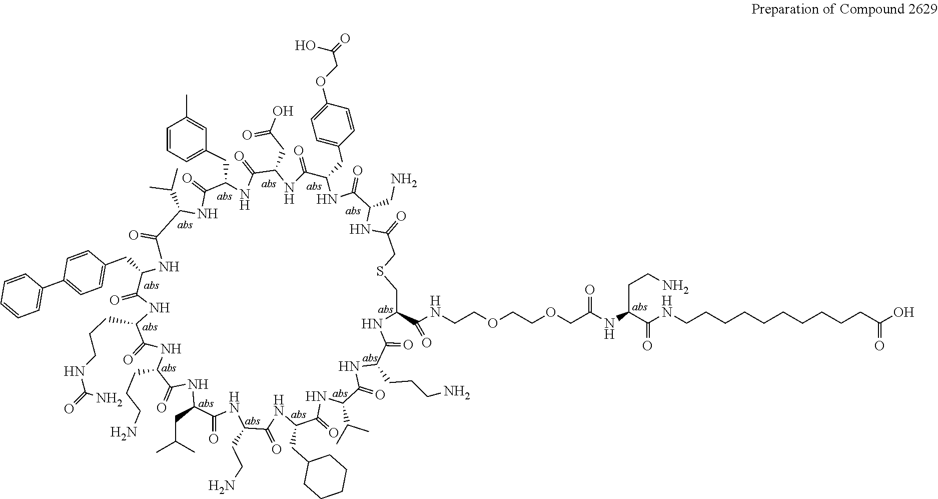 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2629 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.1 mg, and its estimated purity by LCMS analysis was 97.3%. Analysis condition B: Retention time=1.43 min; ESI-MS(+) m/z [M+3H]³⁺: 783.1.

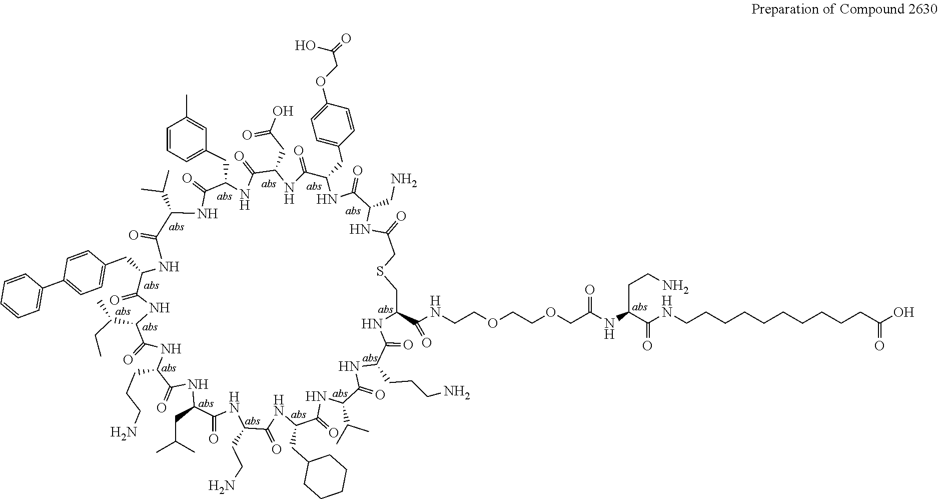 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2630 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.5 mg, and its estimated purity by LCMS analysis was 93.9%. Analysis condition B: Retention time=1.52 min; ESI-MS(+) m/z [M+2H]²⁺: 1152.2.

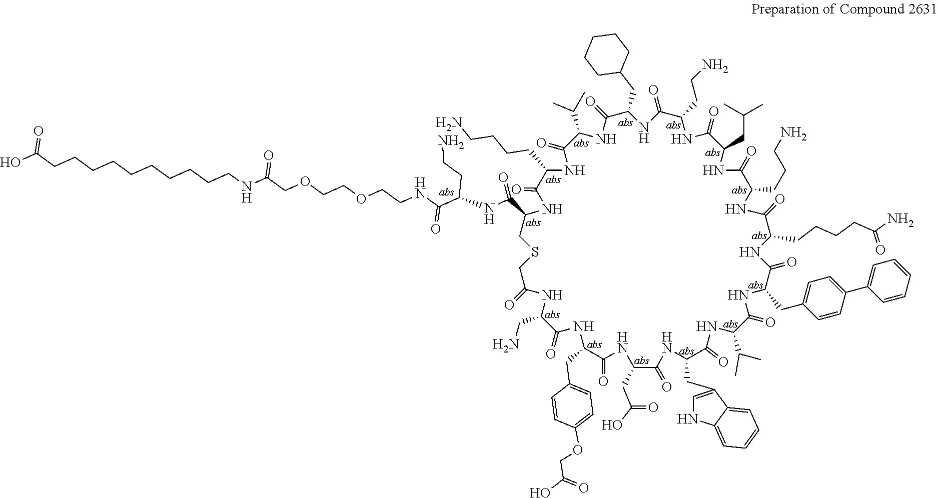 [SMILES: CC(C)C[C@H]1NC(=O)[C@H](CCCN)NC(=O)[C@H](CCCCC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2631 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5 mg, and its estimated purity by LCMS analysis was 98.6%. Analysis condition B: Retention time=1.42 min; ESI-MS(+) m/z [M+3H]³⁺: 796.1.

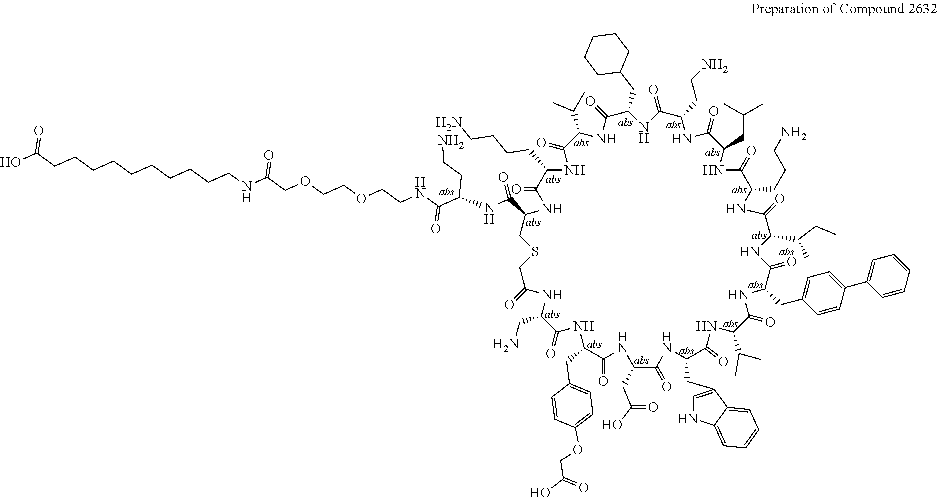 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2632 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.2 mg, and its estimated purity by LCMS analysis was 95%. Analysis condition B: Retention time=1.38 min; ESI-MS(+) m/z [M+3H]³⁺: 781.1.

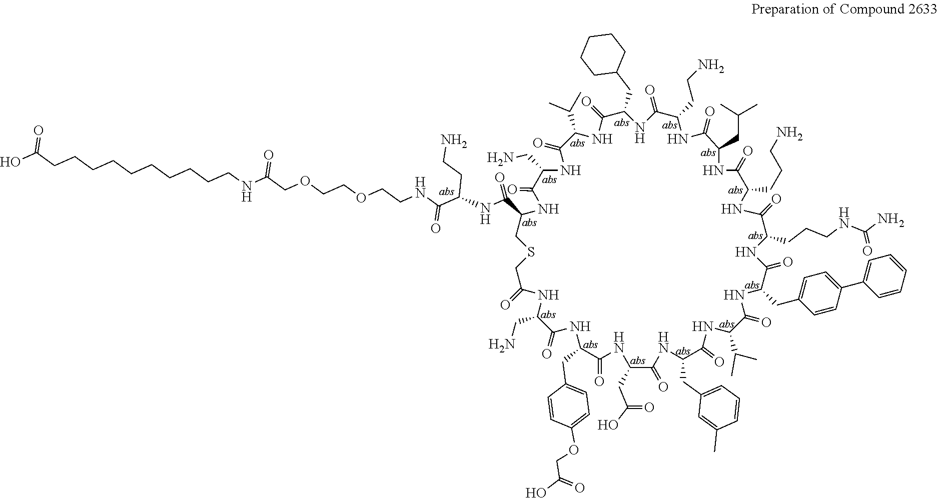 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2633 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.2 mg, and its estimated purity by LCMS analysis was 93.4%. Analysis condition A: Retention time=1.65 min; ESI-MS(+) m/z [M+2H]²⁺: 1160.2.

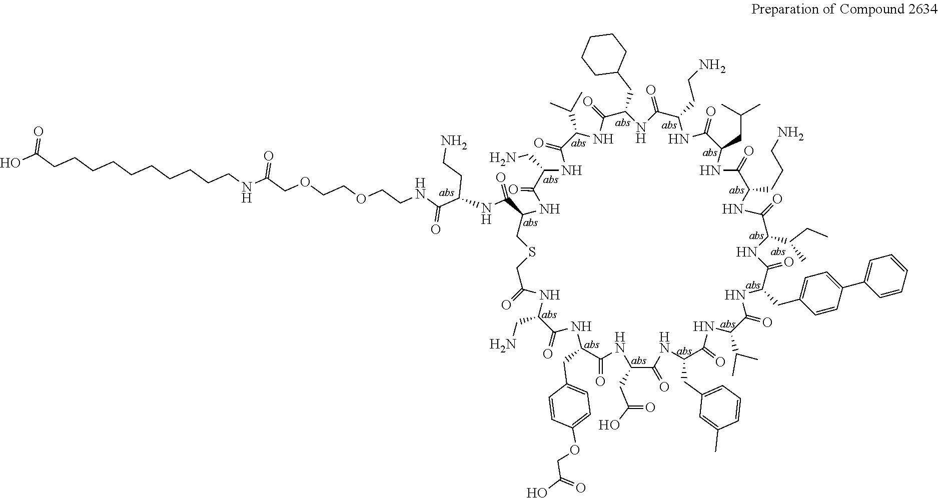 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2634 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.5 mg, and its estimated purity by LCMS analysis was 98.2%. Analysis condition A: Retention time=1.9 min; ESI-MS(+) m/z [M+3H]³⁺: 759.

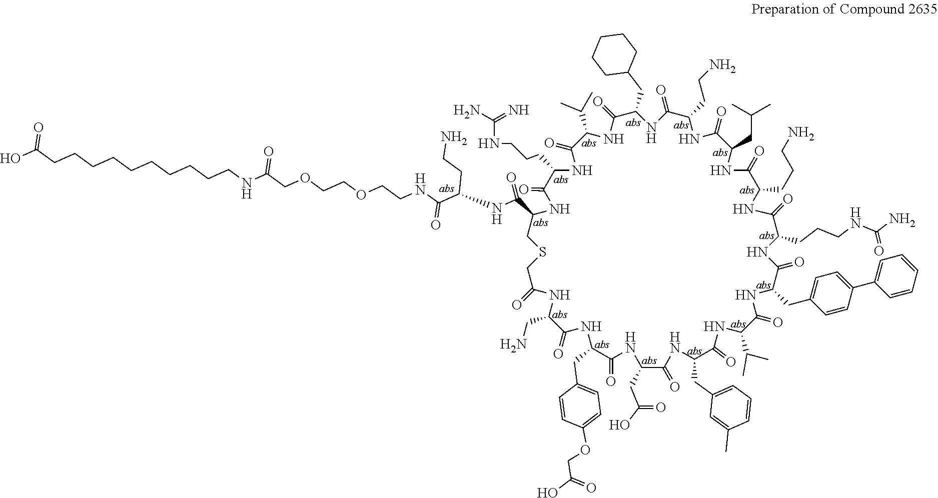 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2635 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-50% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.5 mg, and its estimated purity by LCMS analysis was 92.7%. Analysis condition B: Retention time=1.48 min; ESI-MS(+) m/z [M+3H]³⁺: 797.3.

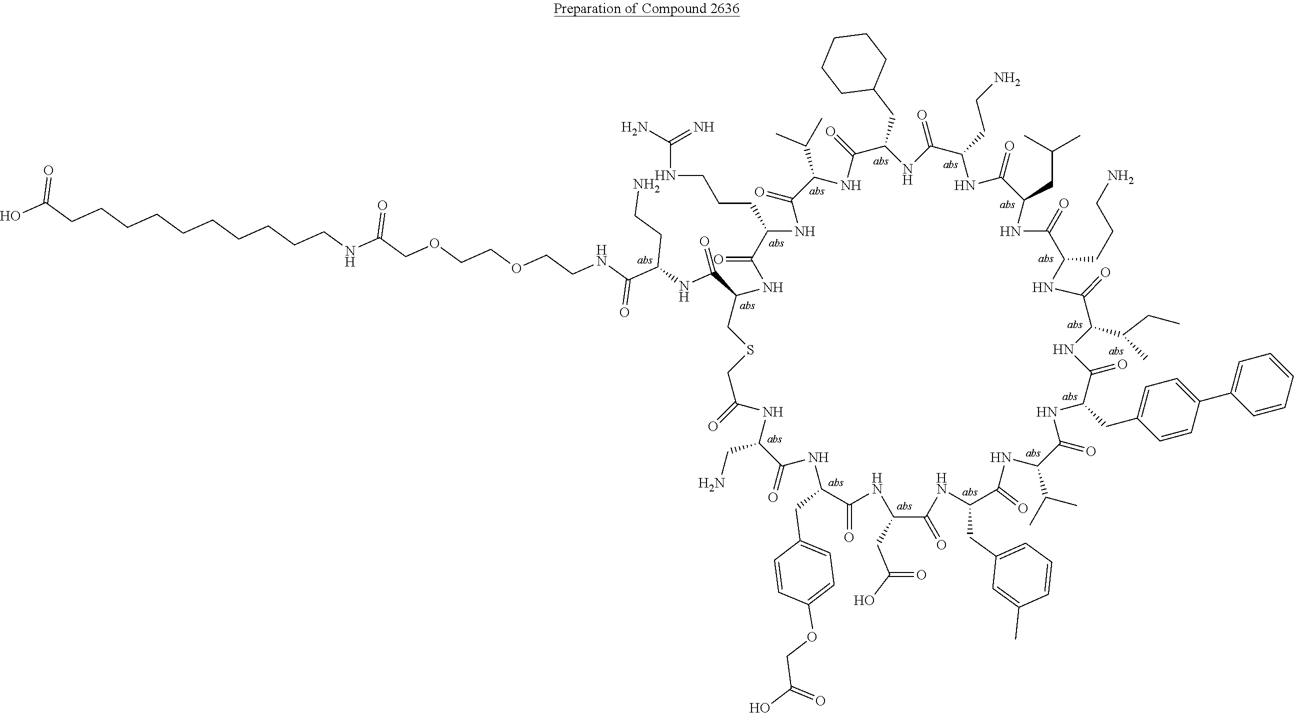 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2636 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.7 mg, and its estimated purity by LCMS analysis was 92.3%. Analysis condition A: Retention time=1.6 min; ESI-MS(+) m/z [M+2H]²⁺: 1172.9.

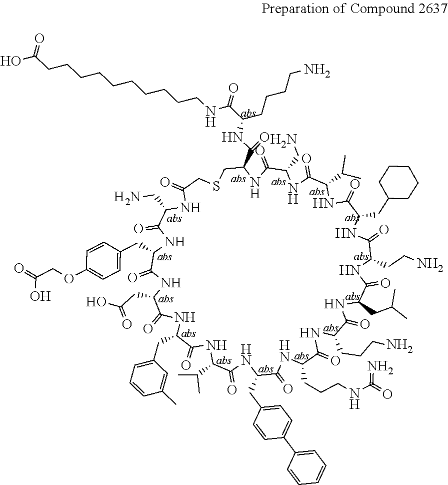

Compound 2637 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.7 mg, and its estimated purity by LCMS analysis was 95.9%. Analysis condition B: Retention time=1.43 min; ESI-MS(+) m/z [M+3H]³⁺: 734.9.

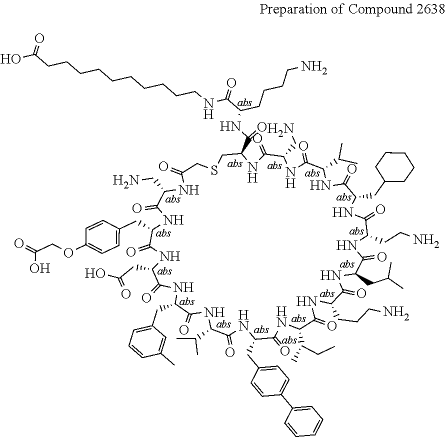 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2638 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 26% B, 26-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.7 mg, and its estimated purity by LCMS analysis was 95.2%. Analysis condition B: Retention time=1.48 min; ESI-MS(+) m/z [M+3H]³⁺: 720.2.

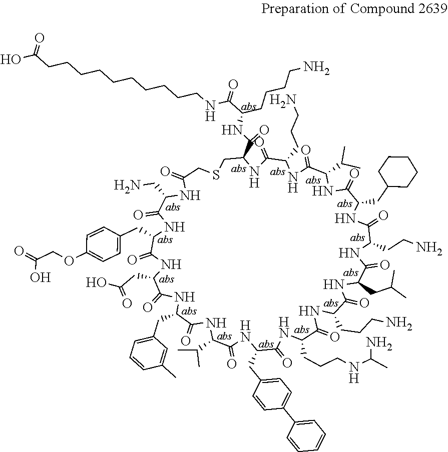 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(C)N)NC(=O)[C@H](Cc3ccc(-c4ccccc4)cc3)NC(=O)[C@H](C(C)C)NC2=O)c1]

Compound 2639 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.2 mg, and its estimated purity by LCMS analysis was 100%. Analysis condition A: Retention time=1.56 min; ESI-MS(+) m/z [M+2H]²⁺: 1115.2.

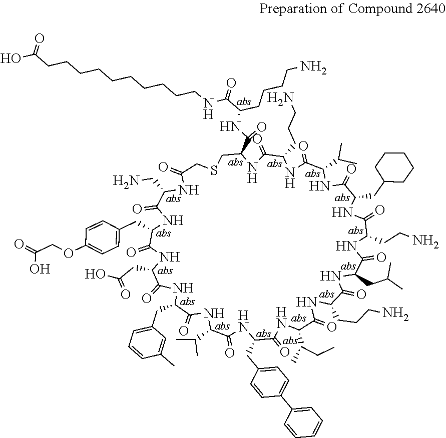 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2640 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 25% B, 25-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.3 mg, and its estimated purity by LCMS analysis was 99.2%. Analysis condition B: Retention time=1.41 min; ESI-MS(+) m/z [M+3H]³⁺: 729.5.

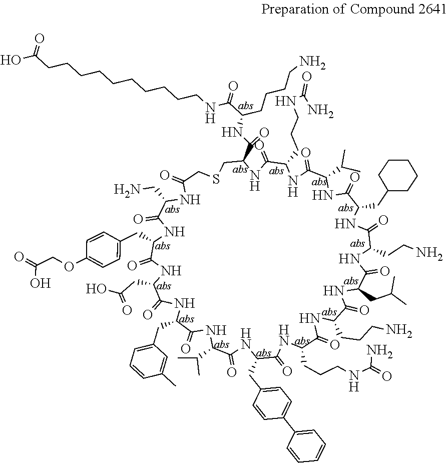 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2641 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate;

Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.3 mg, and its estimated purity by LCMS analysis was 97.8%. Analysis condition B: Retention time=1.43 min; ESI-MS(+) m/z [M+3H]³⁺: 758.1.

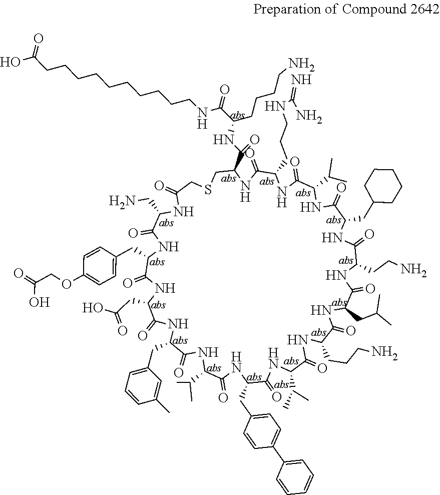

Compound 2642 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.3 mg, and its estimated purity by LCMS analysis was 94.5%. Analysis condition A: Retention time=1.51 min; ESI-MS(+) m/z [M+2H]²⁺: 1114.9.

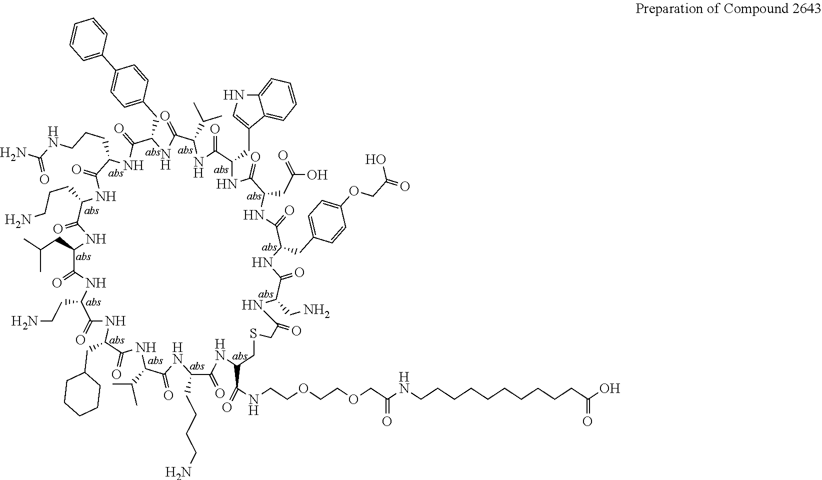

Compound 2643 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 10.3 mg, and its estimated purity by LCMS analysis was 100%. Analysis condition A: Retention time=1.62 min; ESI-MS(+) m/z [M+2H]²⁺: 1144.

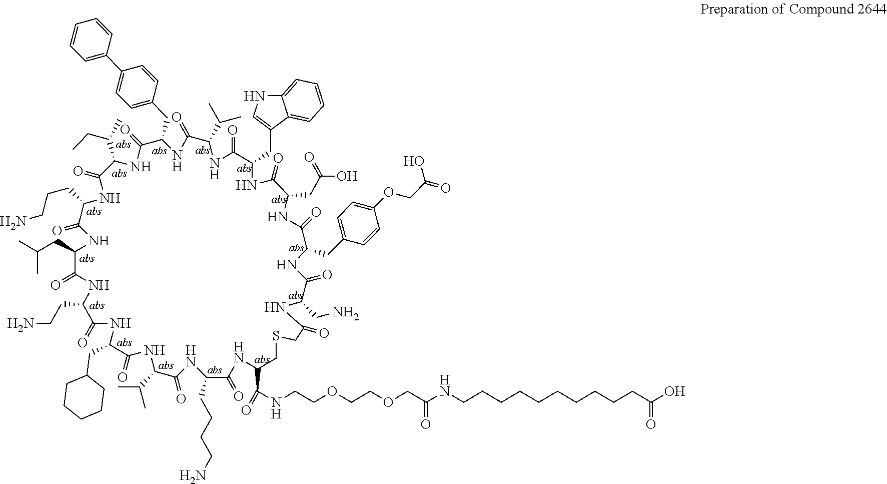

Compound 2644 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.5 mg, and its estimated purity by LCMS analysis was 96.1%. Analysis condition B: Retention time=1.46 min; ESI-MS(+) m/z [M+3H]³⁺: 748.2.

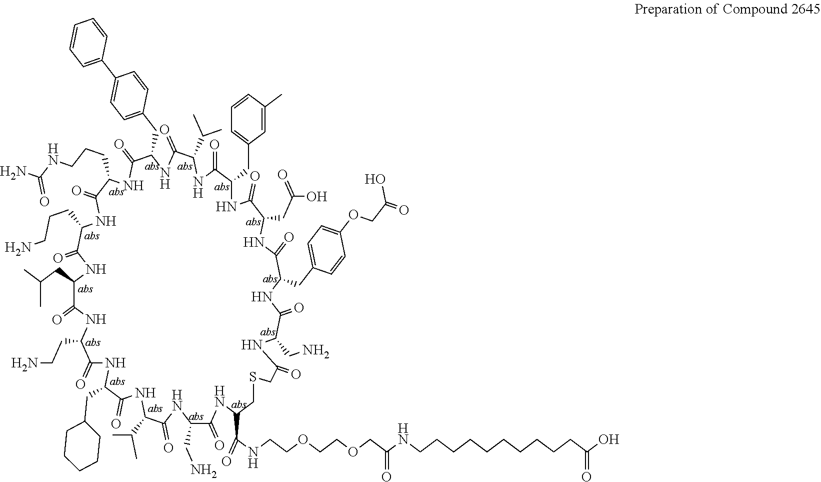

Compound 2645 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 27.2 mg, and its estimated purity by LCMS analysis was 94.4%. Analysis condition B: Retention time=1.6 min; ESI-MS(+) m/z [M+2H]²⁺: 1110.2.

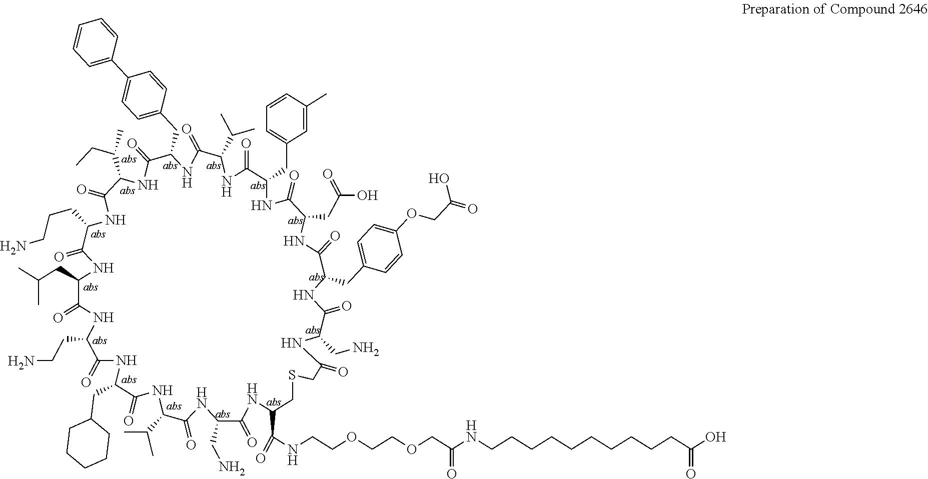

Compound 2646 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 10.2 mg, and its estimated purity by LCMS analysis was 100%. Analysis condition A: Retention time=1.77 min; ESI-MS(+) m/z [M+3H]³⁺: 725.7.

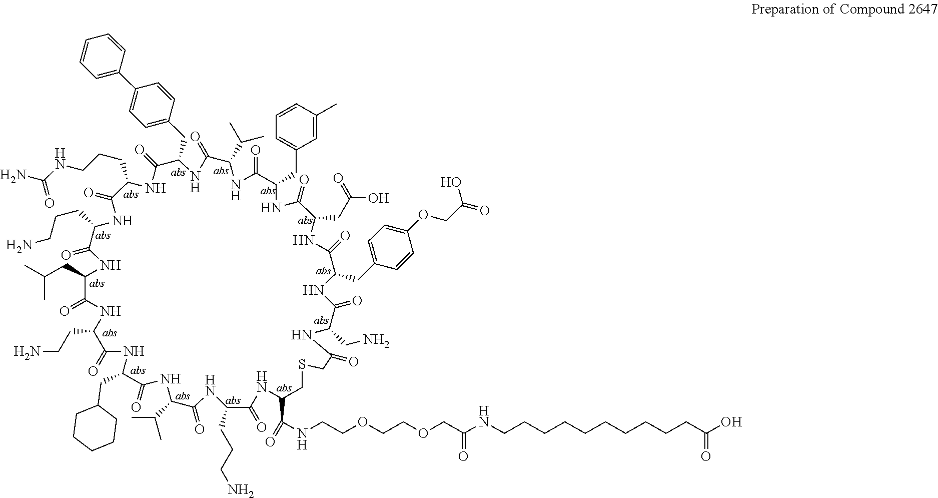

Compound 2647 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15 mg, and its estimated purity by LCMS analysis was 92.8%. Analysis condition B: Retention time=1.49 min; ESI-MS(+) m/z [M+2H]²⁺: 1124.1.

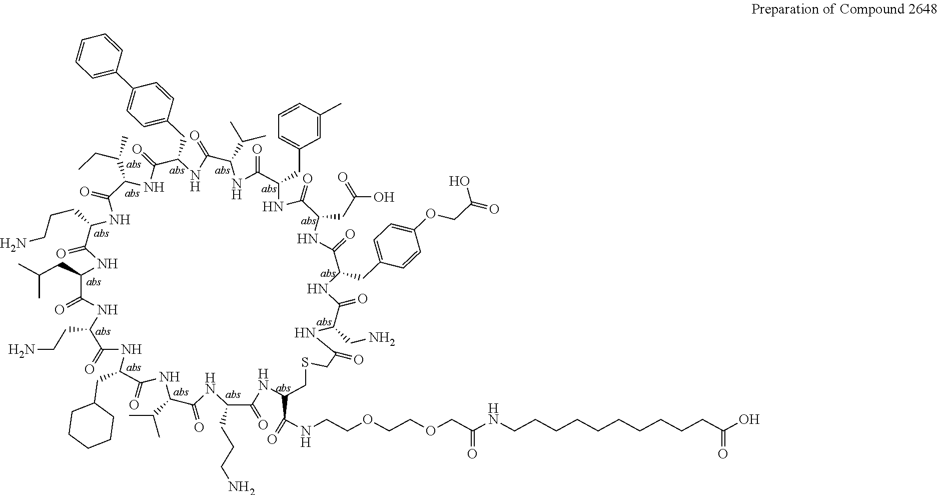

Compound 2648 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.5 mg, and its estimated purity by LCMS analysis was 97.5%. Analysis condition B: Retention time=1.44, 1.5 min; ESI-MS(+) m/z [M+3H]³⁺: 735.04, 735.04.

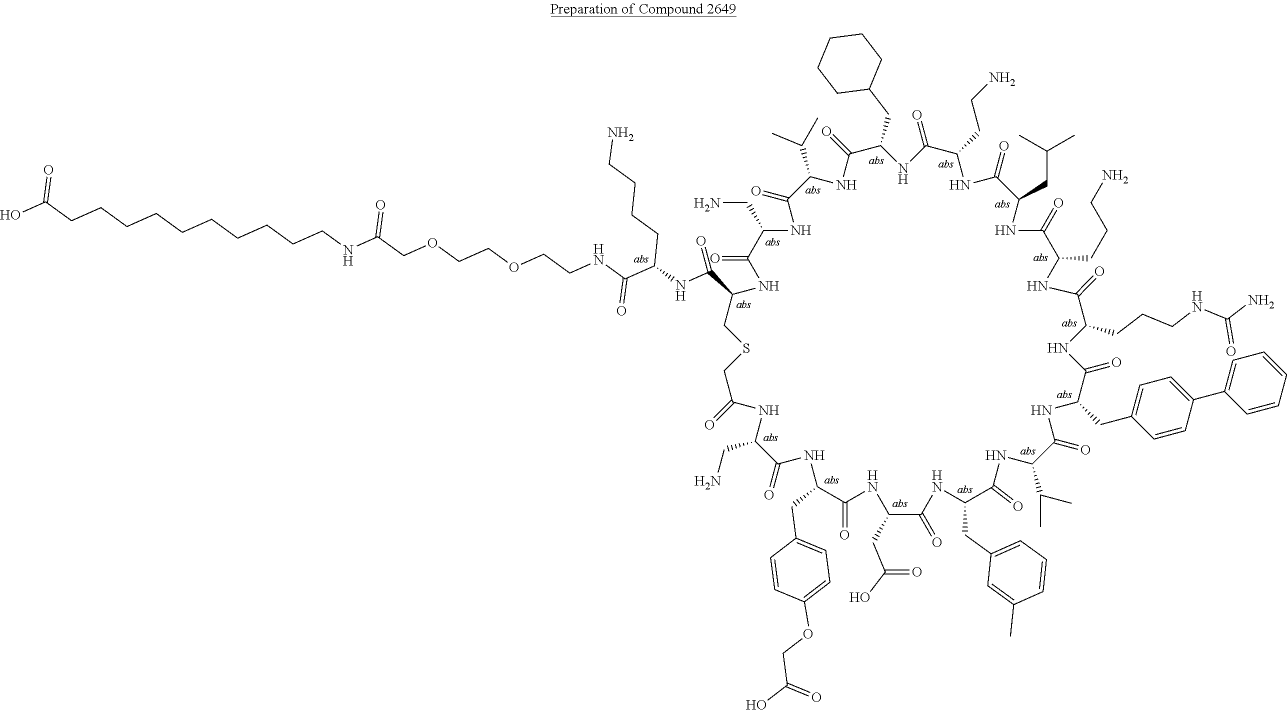 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2649 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.3 mg, and its estimated purity by LCMS analysis was 95.7%. Analysis condition B: Retention time=1.57 min; ESI-MS(+) m/z [M+3H]³⁺: 783.1.

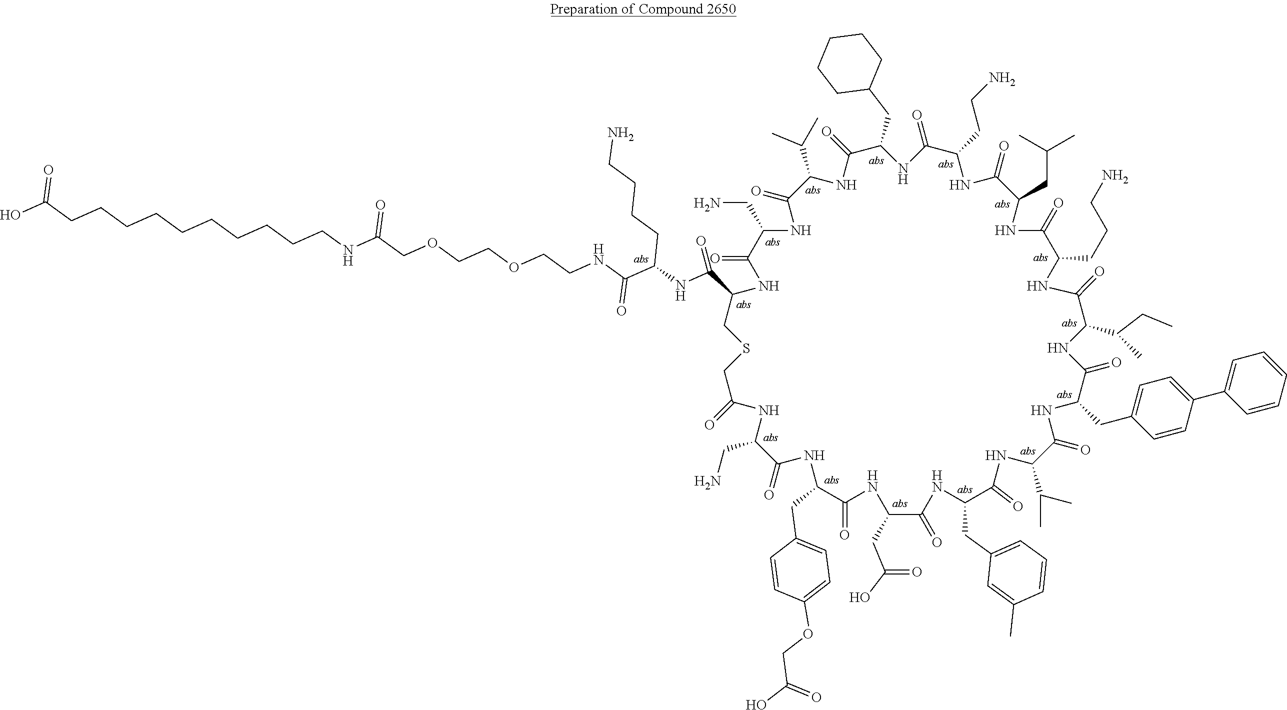 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2650 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13 mg, and its estimated purity by LCMS analysis was 91.7%. Analysis condition B: Retention time=1.57 min; ESI-MS(+) m/z [M+2H]²⁺: 1152.2.

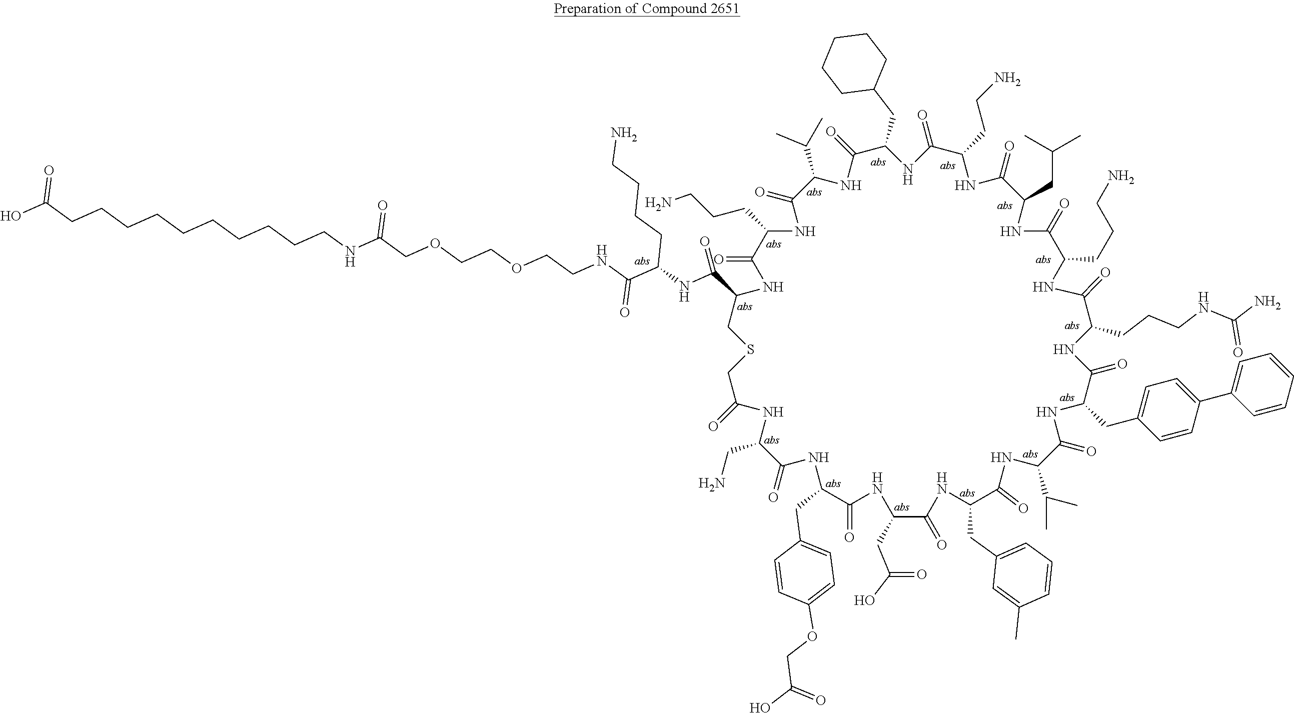 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2651 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 13.2 mg, and its estimated purity by LCMS analysis was 95.3%. Analysis condition B: Retention time=1.5 min; ESI-MS(+) m/z [M+2H]²⁺: 1188.

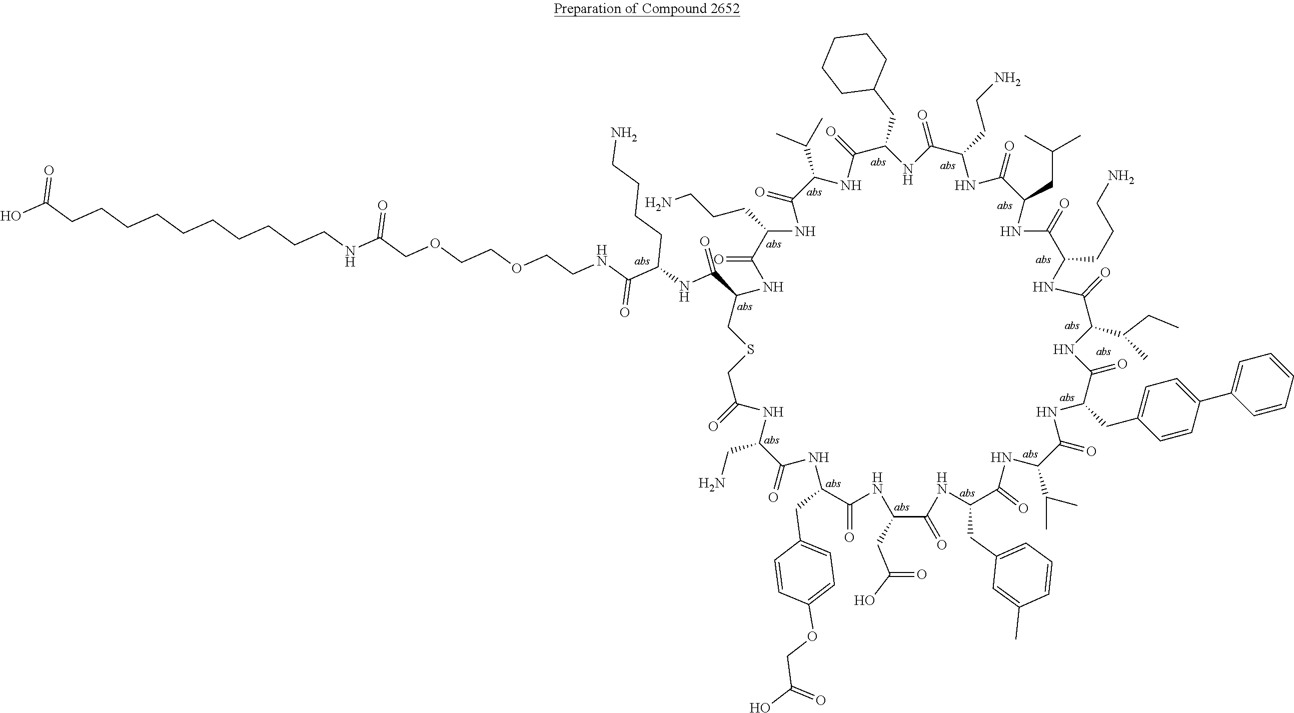 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2652 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.5 mg, and its estimated purity by LCMS analysis was 98.4%. Analysis condition A: Retention time=2.03 min; ESI-MS(+) m/z [M+3H]³⁺: 778.

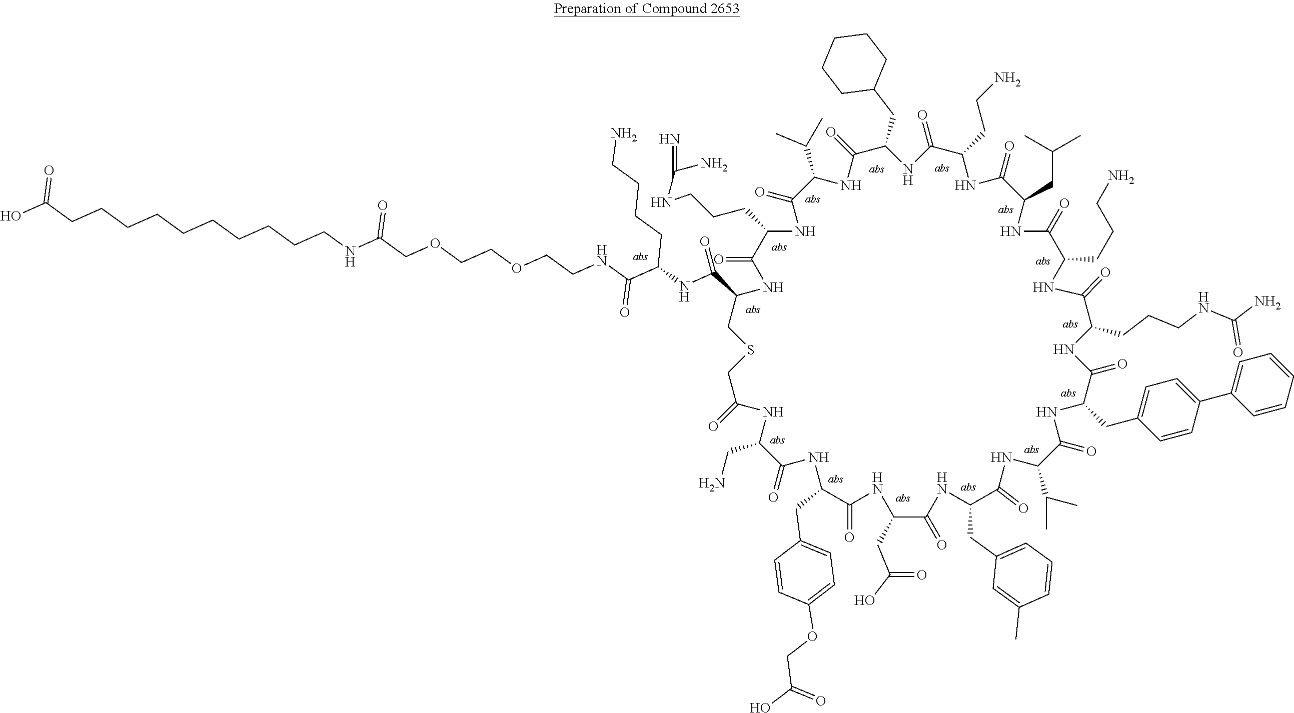 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2653 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.4 mg, and its estimated purity by LCMS analysis was 96.1%. Analysis condition B: Retention time=1.49 min; ESI-MS(+) m/z [M+2H]²⁺: 1209.2.

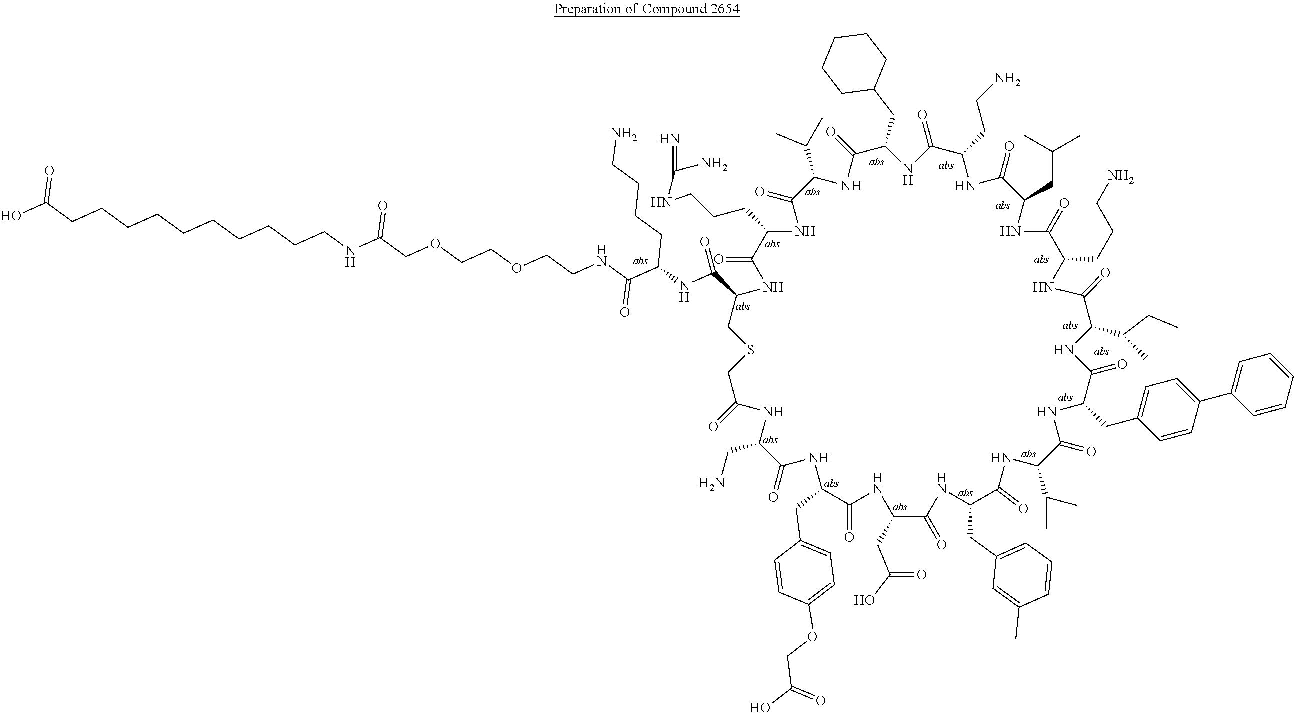 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2654 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.3 mg, and its estimated purity by LCMS analysis was 98.5%. Analysis condition B: Retention time=1.4 min; ESI-MS(+) m/z [M+3H]³⁺: 792.1.

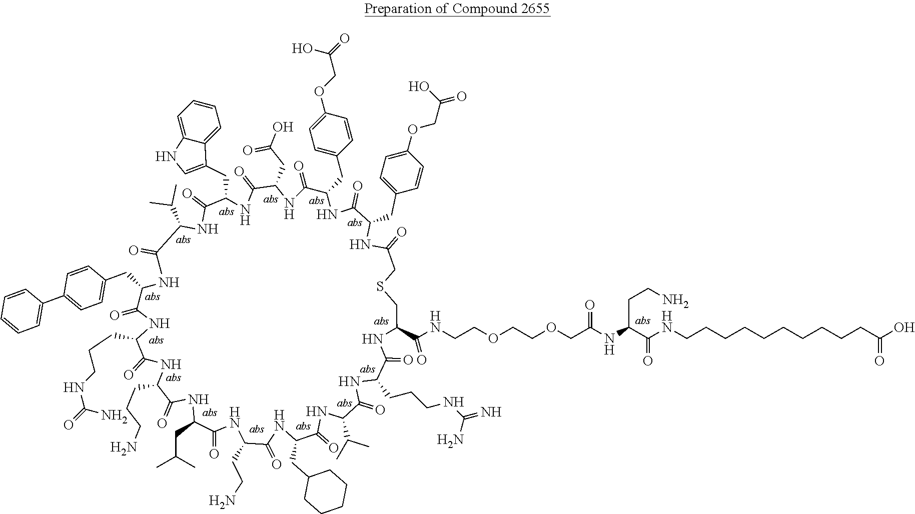 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2655 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.5 mg, and its estimated purity by LCMS analysis was 92.1%. Analysis condition B: Retention time=1.58 min; ESI-MS(+) m/z [M+3H]³⁺: 850.1.

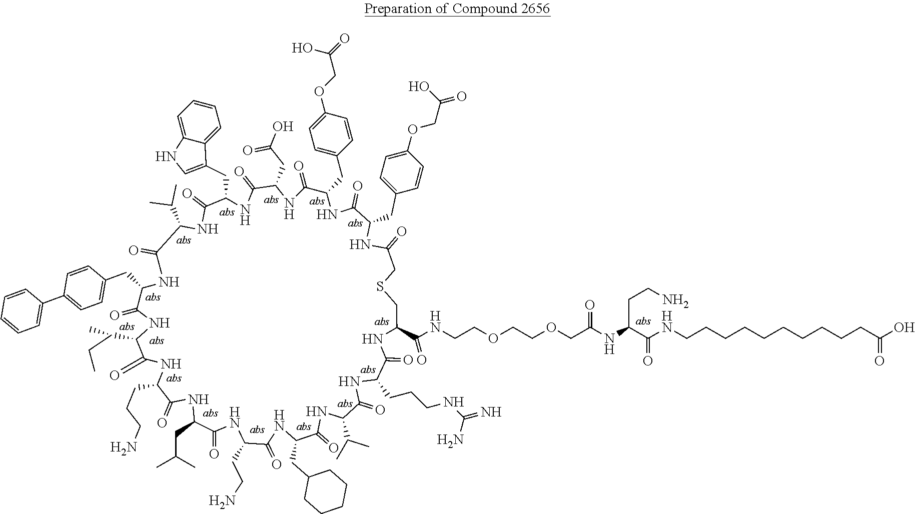 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2656 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5 mg, and its estimated purity by LCMS analysis was 92.8%. Analysis condition A: Retention time=1.64 min; ESI-MS(+) m/z [M+2H]²⁺: 1253.2.

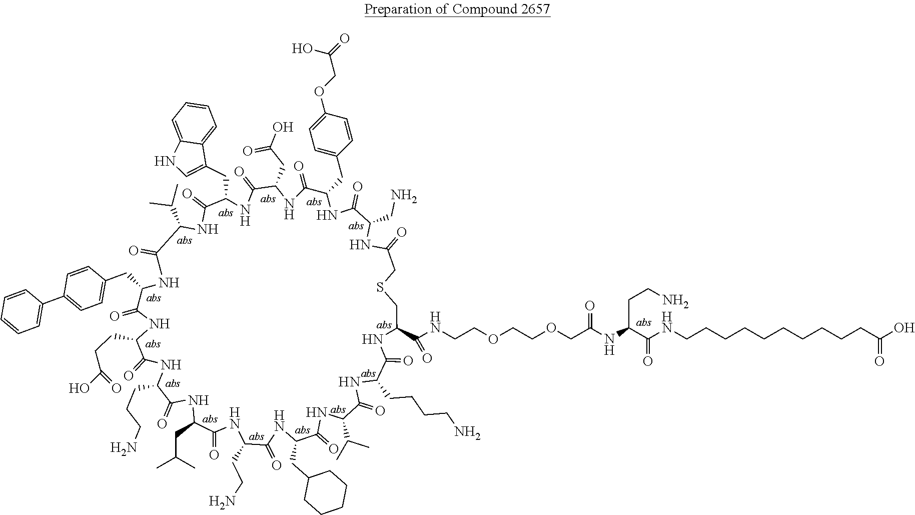 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2657 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.7 mg, and its estimated purity by LCMS analysis was 97.5%. Analysis condition B: Retention time=1.42 min; ESI-MS(+) m/z [M+3H]³⁺: 787.1.

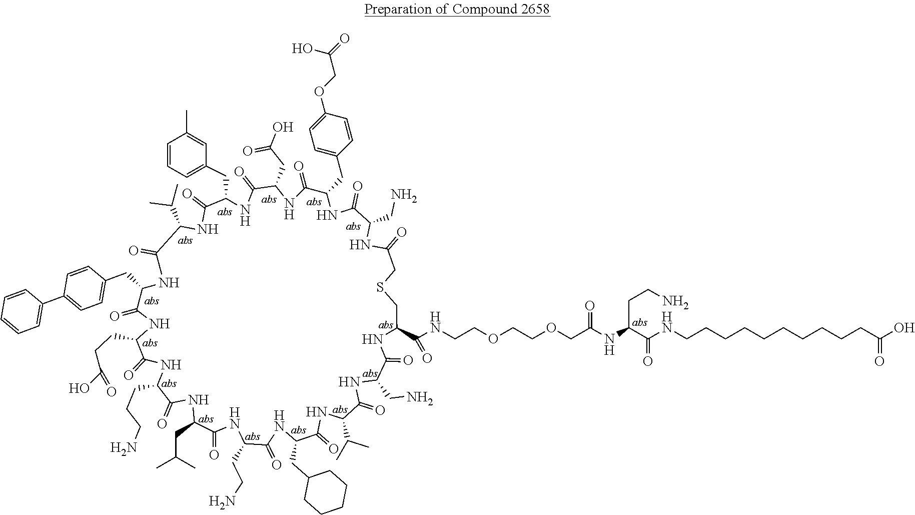 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2658 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 11 mg, and its estimated purity by LCMS analysis was 93.2%. Analysis condition B: Retention time=1.55 min; ESI-MS(+) m/z [M+2H]²⁺: 1145.8.

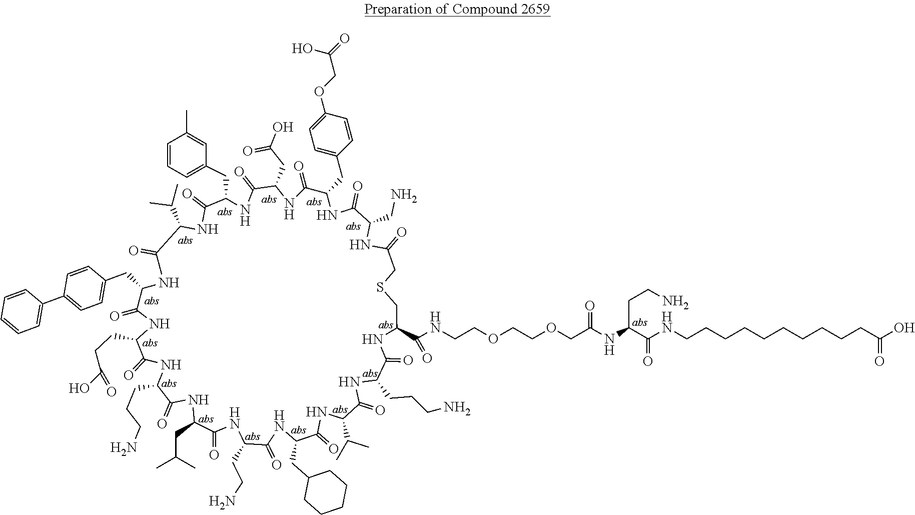 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2659 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.2 mg, and its estimated purity by LCMS analysis was 100%. Analysis condition B: Retention time=1.45 min; ESI-MS(+) m/z [M+2H]²⁺: 1159.8.

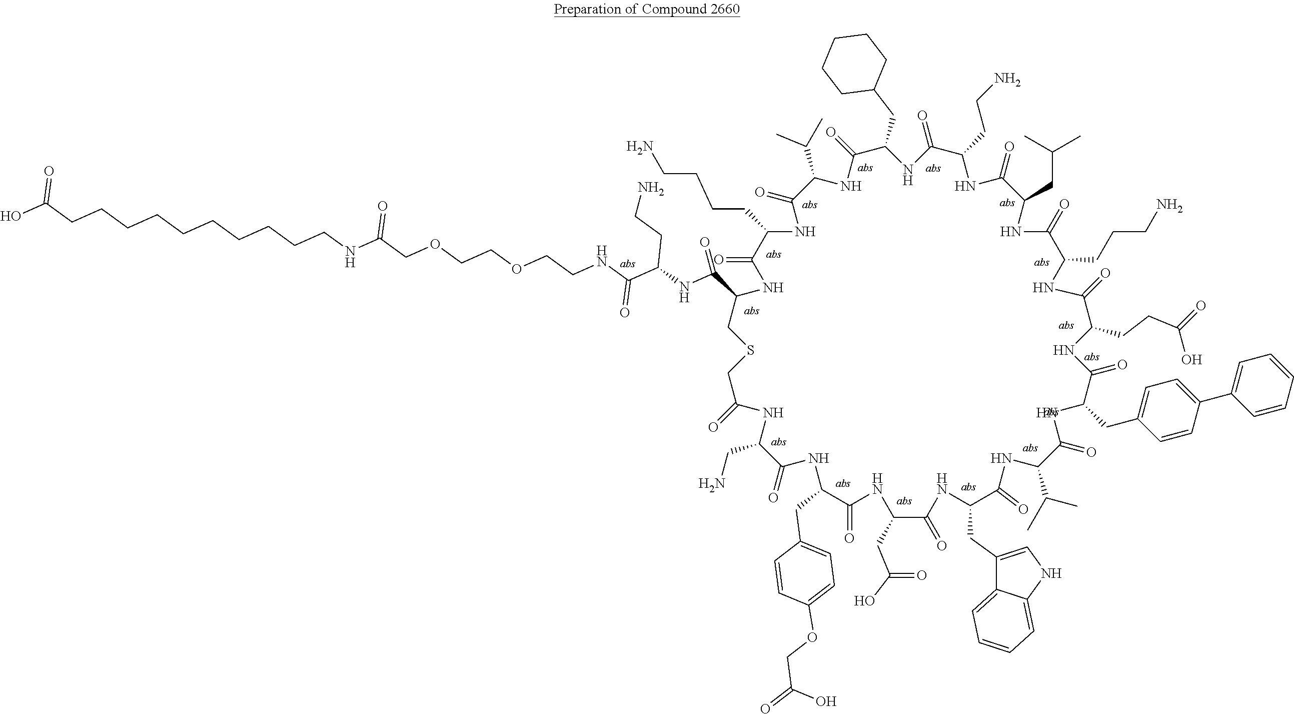 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2660 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 8.4 mg, and its estimated purity by LCMS analysis was 95.1%. Analysis condition B: Retention time=1.52 min; ESI-MS(+) m/z [M+3H]³⁺: 787.1.

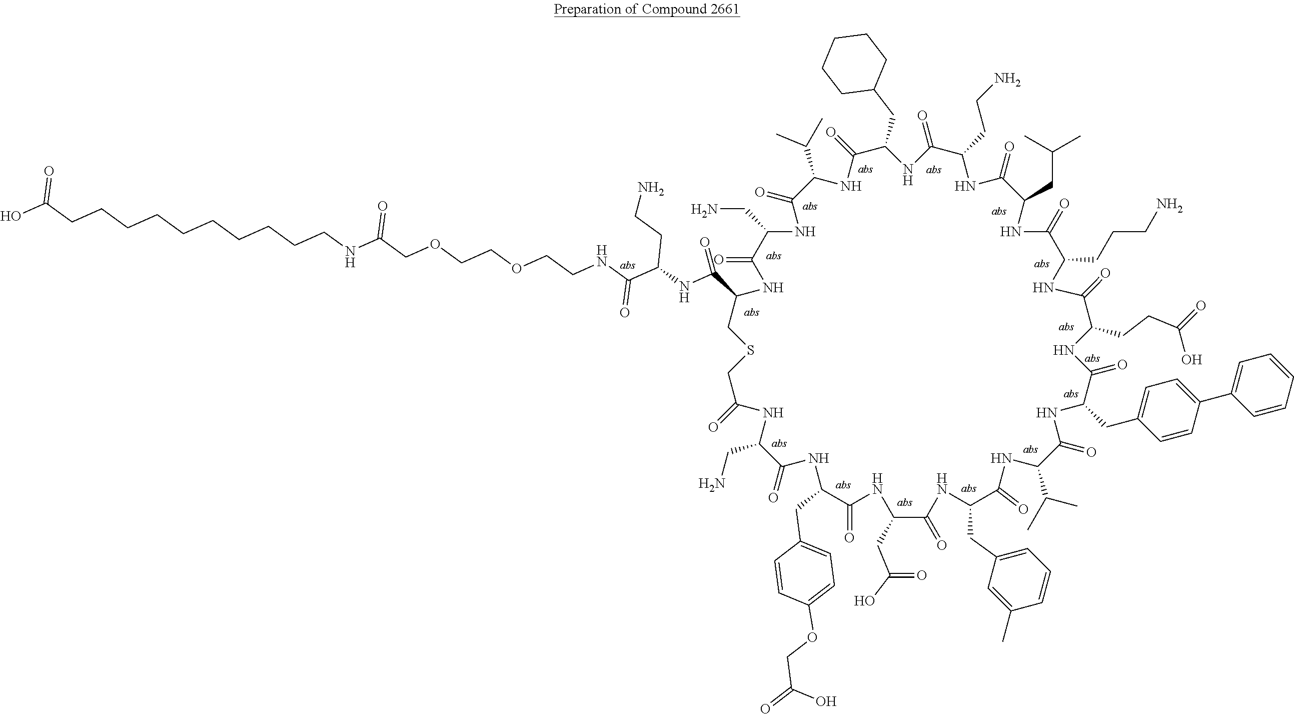 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2661 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 9.9 mg, and its estimated purity by LCMS analysis was 88.2%. Analysis condition B: Retention time=1.55 min; ESI-MS(+) m/z [M+2H]²⁺: 1146.

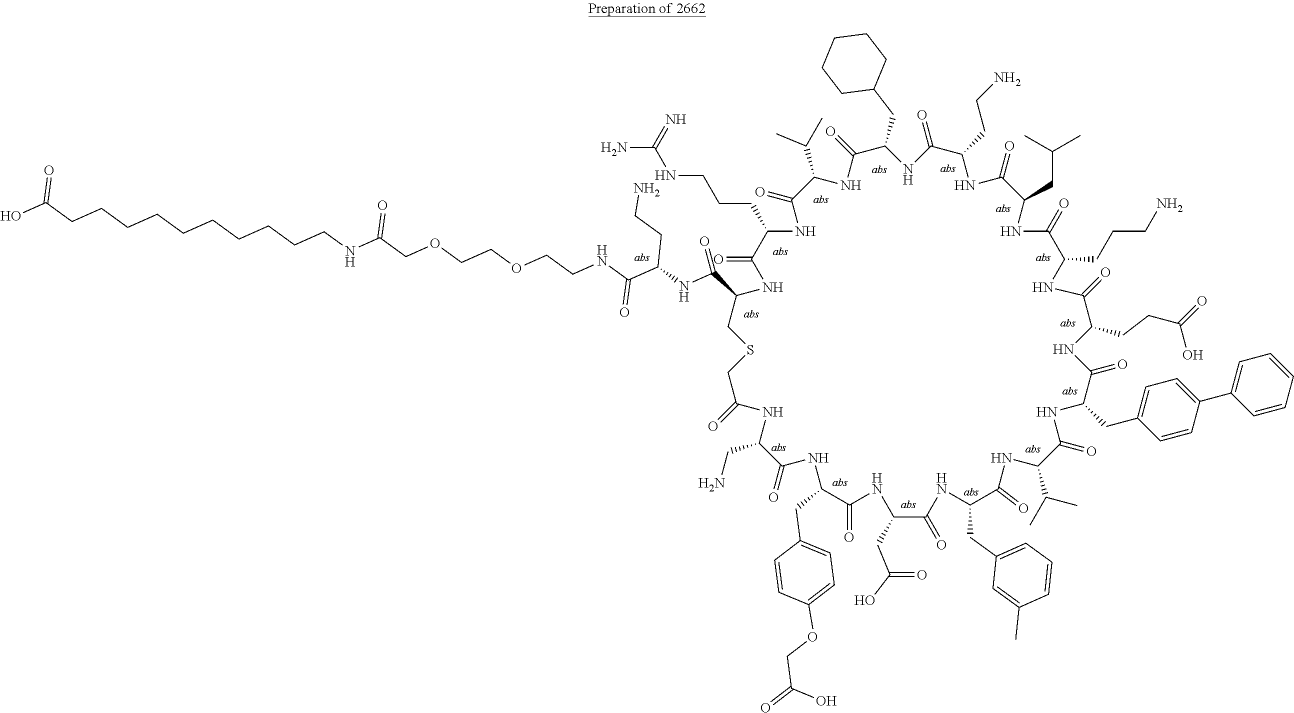 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2662 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.8 mg, and its estimated purity by LCMS analysis was 100%. Analysis condition B: Retention time=1.49 min; ESI-MS(+) m/z [M+2H]²⁺: 1181.2.

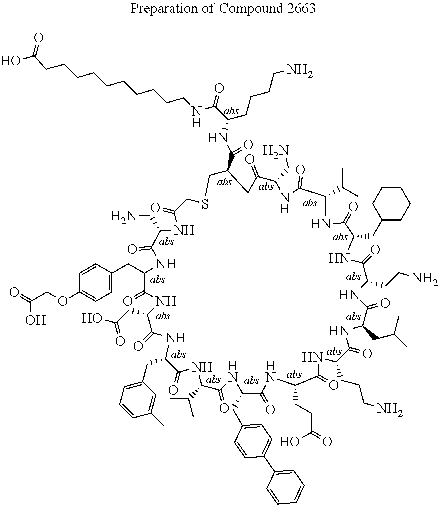 [SMILES: Cc1cccc(C[C@@H]2NC(=O)[C@H](CC(=O)O)NC(=O)C(Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)CC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2663 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15 mg, and its estimated purity by LCMS analysis was 100%. Analysis condition B: Retention time=1.47 min; ESI-MS(+) m/z [M+2H]²⁺: 1087.2.

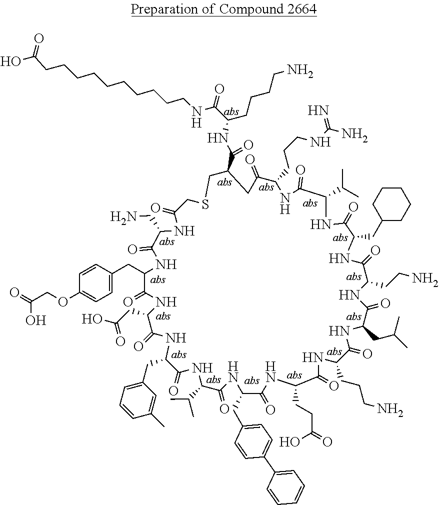 [SMILES: Cc1cccc(C[C@@H]2NC(=O)[C@H](CC(=O)O)NC(=O)C(Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)CC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2664 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.1 mg, and its estimated purity by LCMS analysis was 94.2%. Analysis condition B: Retention time=1.46 min; ESI-MS(+) m/z [M+3H]³⁺: 749.1.

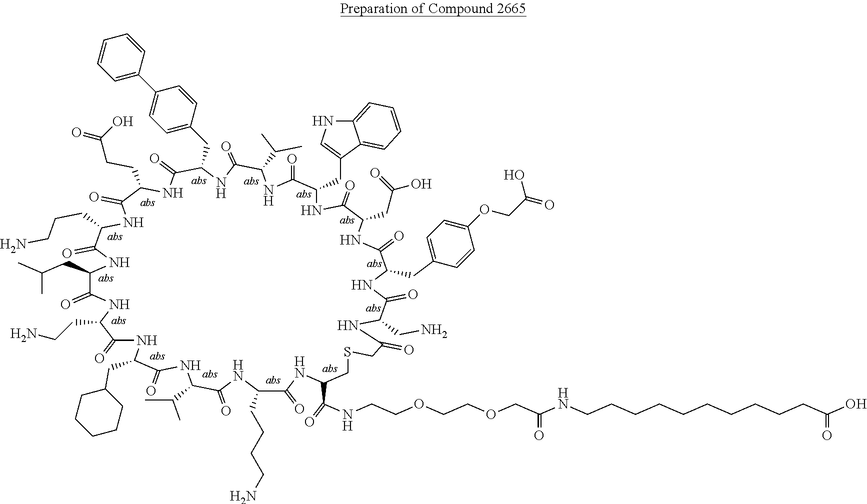

Compound 2665 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.4 mg, and its estimated purity by LCMS analysis was 99.1%. Analysis condition A: Retention time=1.57 min; ESI-MS(+) m/z [M+2H]²⁺: 1129.9.

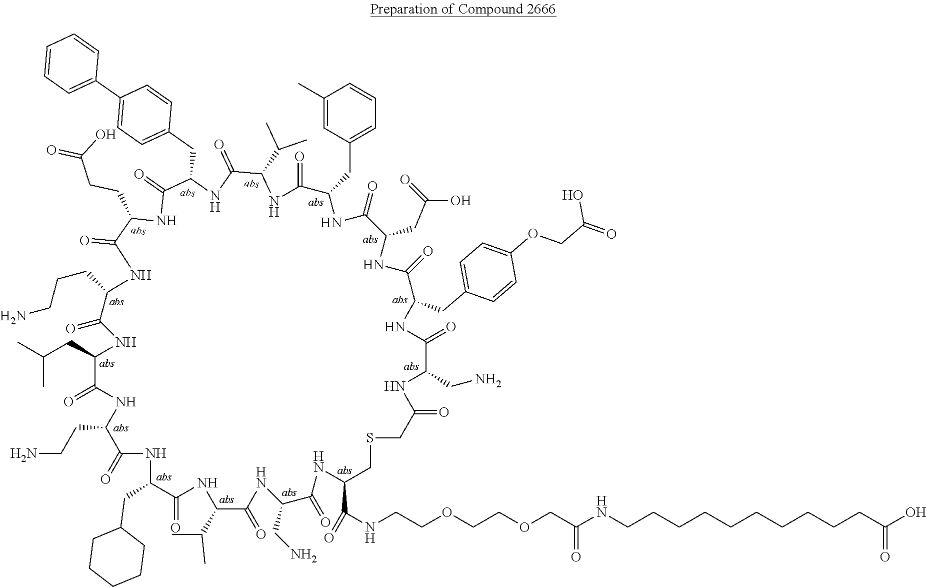 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2666 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 28.5 mg, and its estimated purity by LCMS analysis was 95.3%. Analysis condition B: Retention time=1.6 min; ESI-MS(+) m/z [M+3H]³⁺: 731.

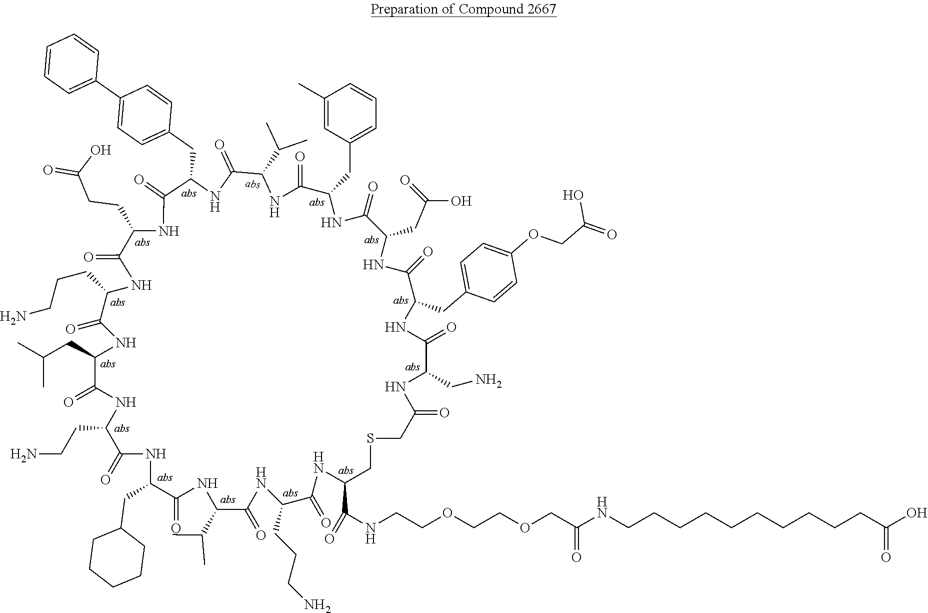 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2667 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 18 mg, and its estimated purity by LCMS analysis was 93.1%. Analysis condition B: Retention time=1.58, 1.62 min; ESI-MS(+) m/z [M+2H]²⁺: 1109.82, 1109.82.

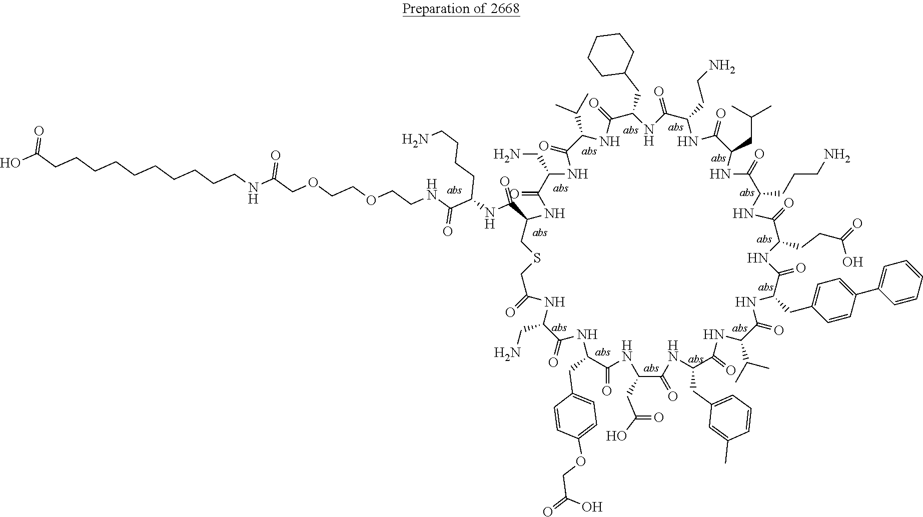 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2668 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.8 mg, and its estimated purity by LCMS analysis was 93.1%. Analysis condition B: Retention time=1.54 min; ESI-MS(+) m/z [M+2H]²⁺: 1160.1.

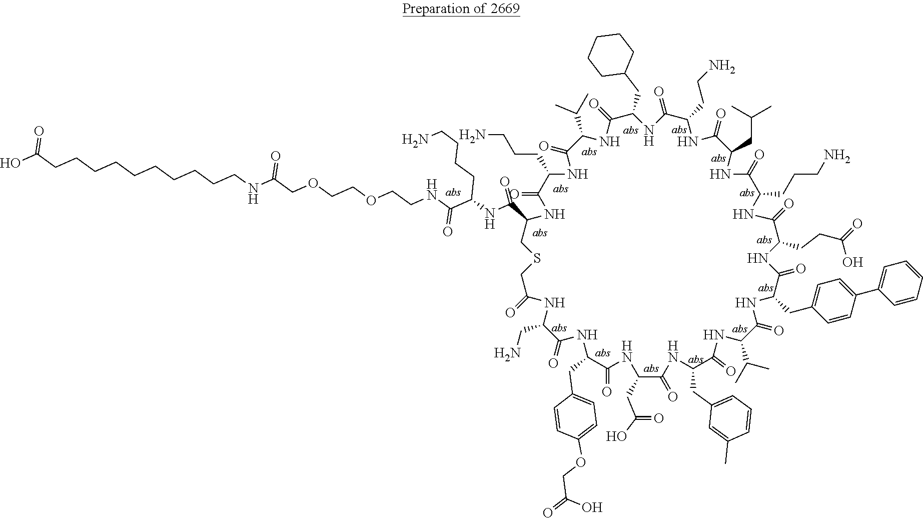 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2669 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 11.4 mg, and its estimated purity by LCMS analysis was 96.1%. Analysis condition A: Retention time=1.51 min; ESI-MS(+) m/z [M+2H]²⁺: 1174.1.

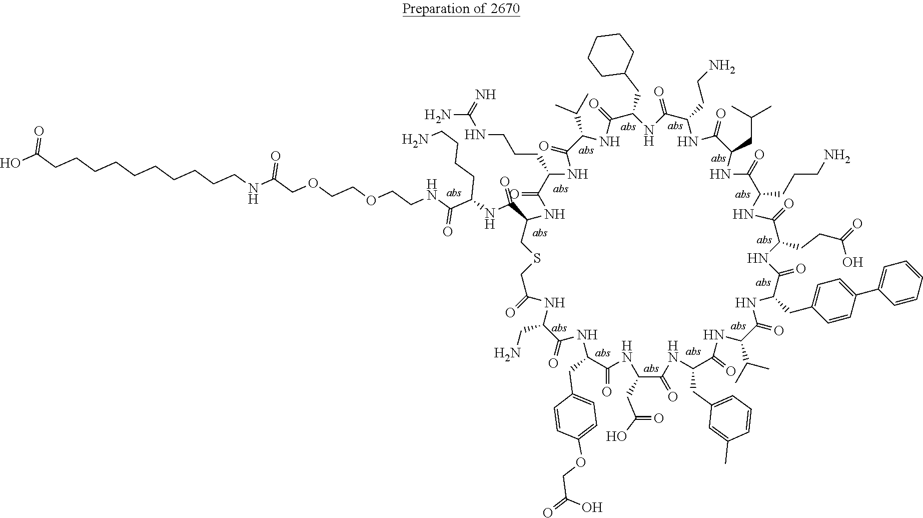 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2670 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at centrifugal evaporation. The yield of the product was 4.7 mg, and its estimated purity by LCMS analysis was 100%. Analysis condition A: Retention time=1.41 min; ESI-MS(+) m/z [M+3H]³⁺: 797.2.

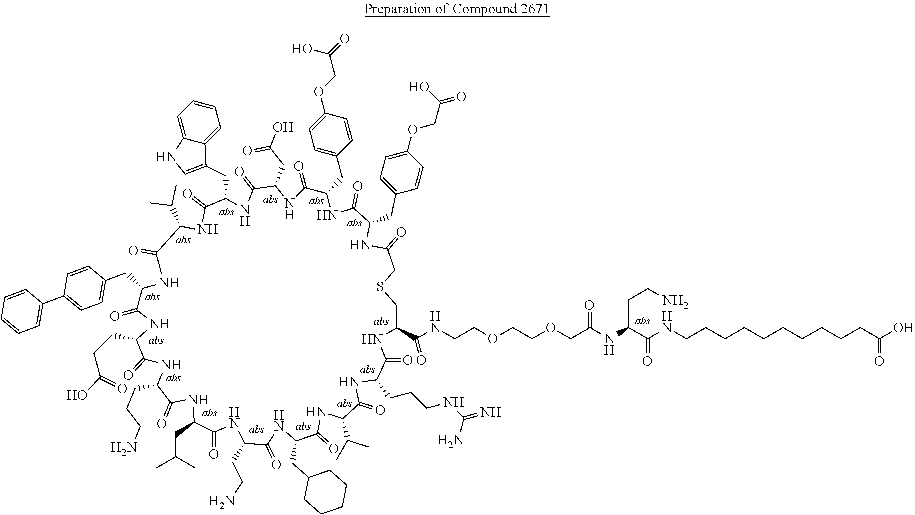 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCN)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2671 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.3 mg, and its estimated purity by LCMS analysis was 96.6%. Analysis condition B: Retention time=1.59 min; ESI-MS(+) m/z [M+3H]³⁺: 841.1.

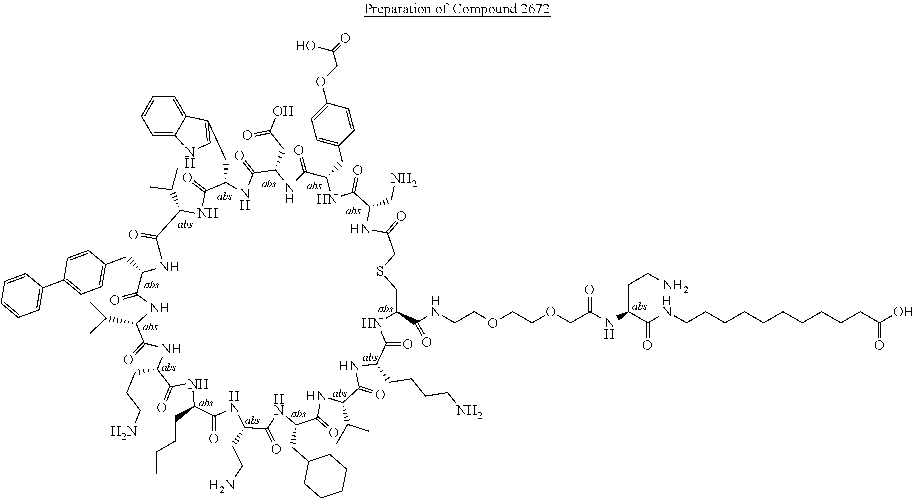 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2672 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.2 mg, and its estimated purity by LCMS analysis was 95.3%. Analysis condition A: Retention time=1.49 min; ESI-MS(+) m/z [M+2H]²⁺: 1164.1.

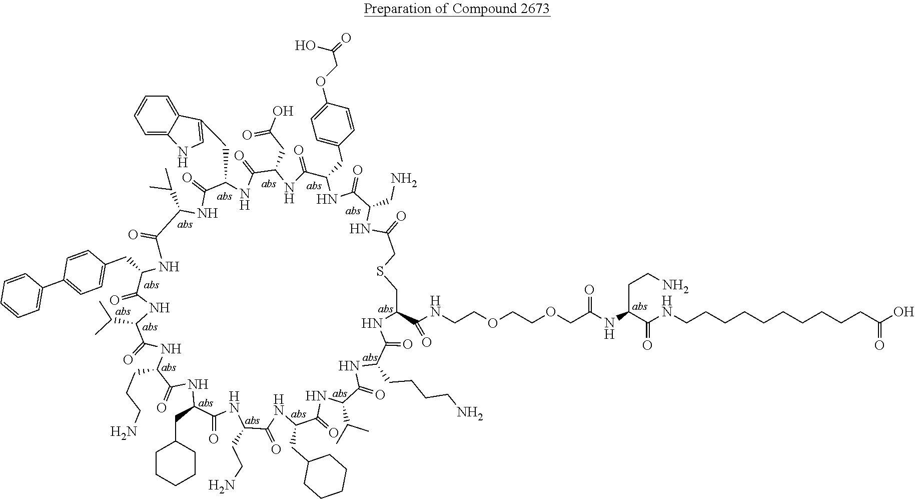 [SMILES: CC(C)[C@@H]1NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCCN)NC1=O]

Compound 2673 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.4 mg, and its estimated purity by LCMS analysis was 92.9%. Analysis condition A: Retention time=1.58 min; ESI-MS(+) m/z [M+3H]³⁺: 790.2.

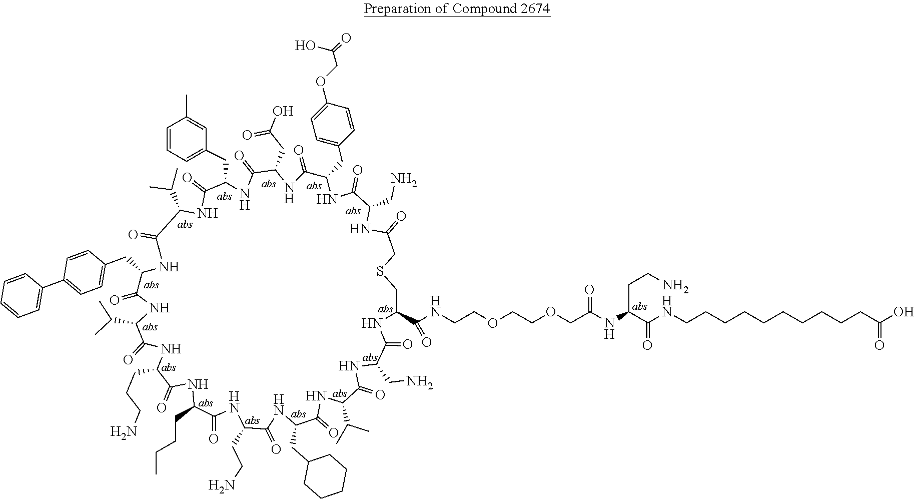 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2674 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.3 mg, and its estimated purity by LCMS analysis was 97.9%. Analysis condition B: Retention time=1.45 min; ESI-MS(+) m/z [M+3H]³⁺: 754.1.

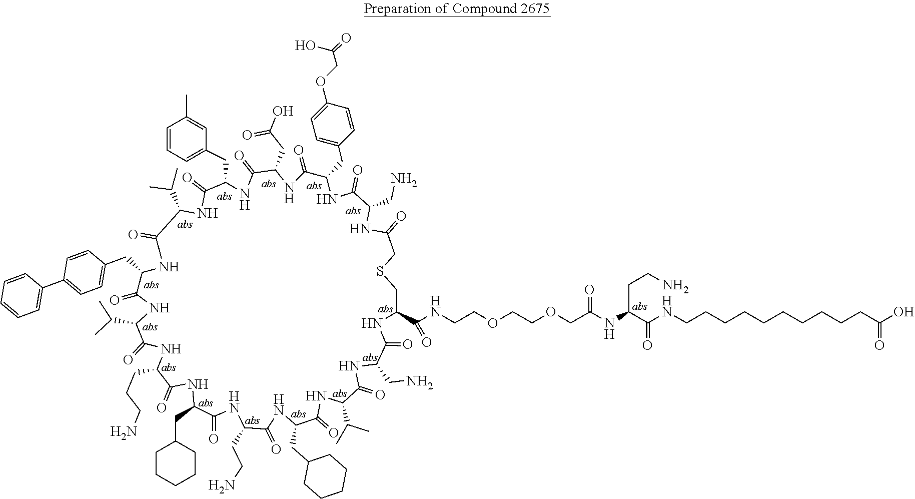 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC3CCCCC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2675 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.9 mg, and its estimated purity by LCMS analysis was 100%. Analysis condition B: Retention time=1.59 min; ESI-MS(+) m/z [M+2H]²⁺: 1151.2.

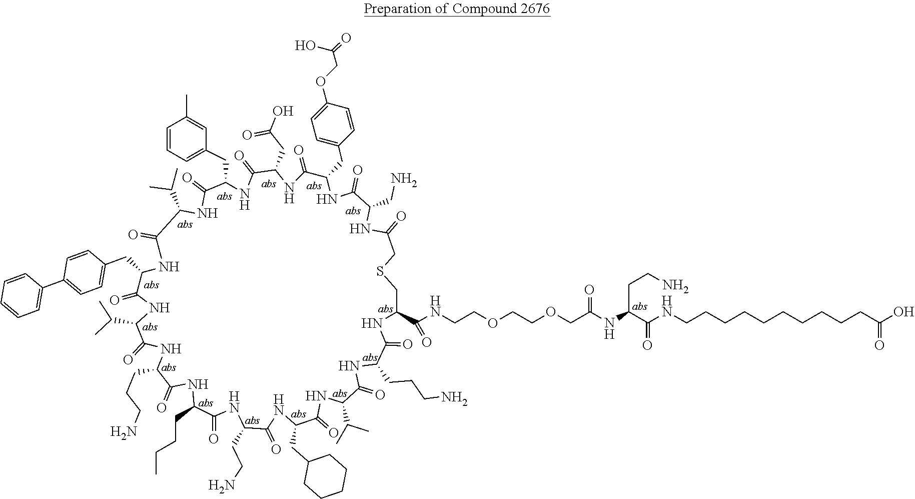 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2676 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.8 mg, and its estimated purity by LCMS analysis was 96.5%. Analysis condition A: Retention time=1.57 min; ESI-MS(+) m/z [M+2H]²⁺: 1145.1.

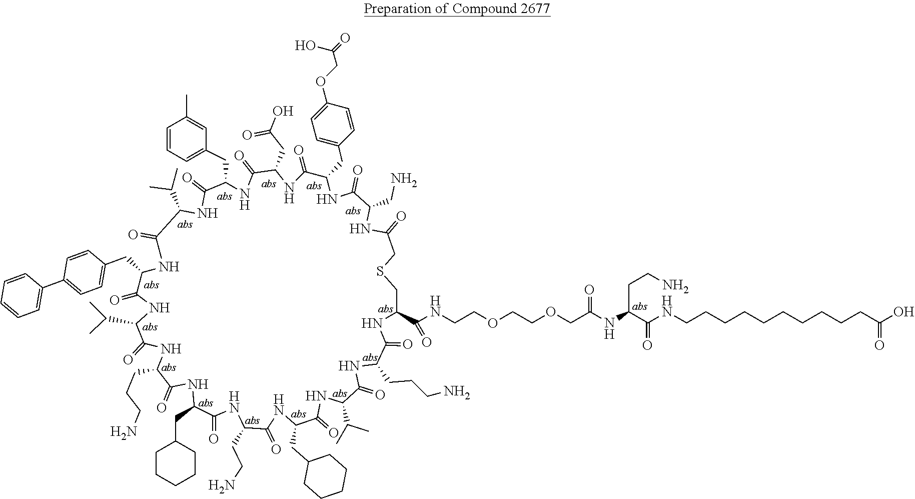 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC3CCCCC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2677 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.3 mg, and its estimated purity by LCMS analysis was 98.9%. Analysis condition B: Retention time=1.63 min; ESI-MS(+) m/z [M+3H]³⁺: 776.9.

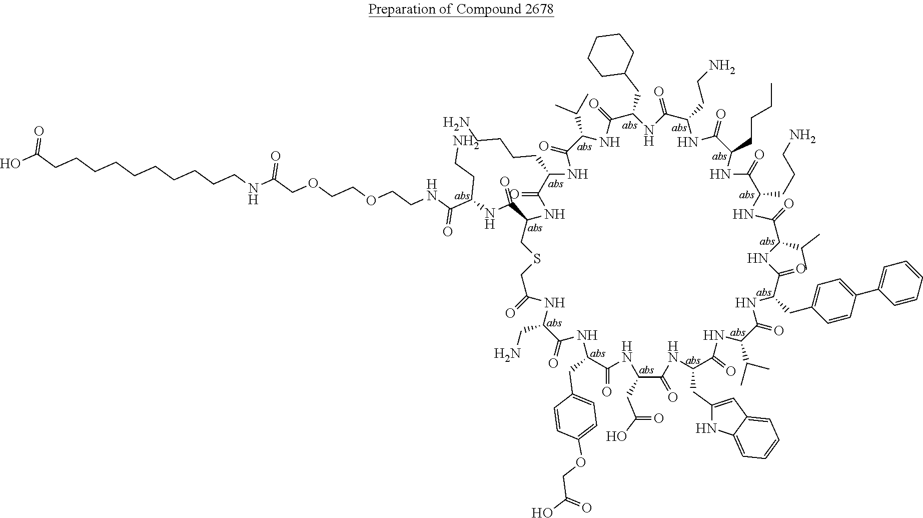 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cc3ccccc3[nH]2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 2678 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-52% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.7 mg, and its estimated purity by LCMS analysis was 99.2%. Analysis condition BA: Retention time=1.37 min; ESI-MS(+) m/z [M+3H]³⁺: 776.9.

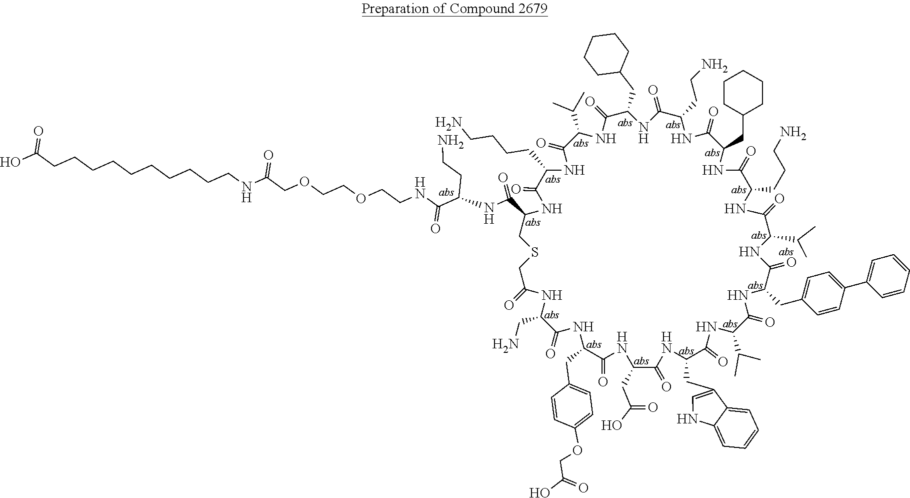 [SMILES: CC(C)[C@@H]1NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCCN)NC1=O]

Compound 2679 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 6.2 mg, and its estimated purity by LCMS analysis was 95.1%. Analysis condition A: Retention time=1.63 min; ESI-MS(+) m/z [M+3H]³⁺: 790.1.

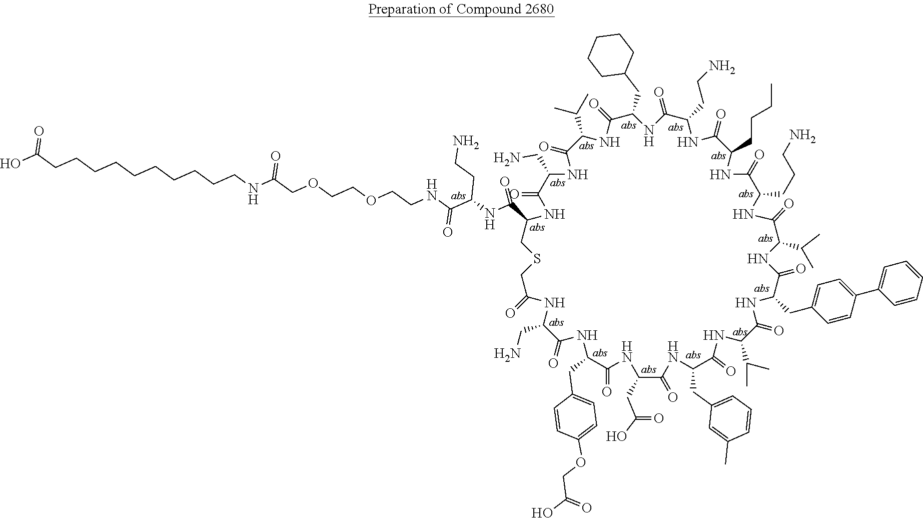 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2680 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.1 mg, and its estimated purity by LCMS analysis was 92.5%. Analysis condition B: Retention time=1.62 min; ESI-MS(+) m/z [M+2H]²⁺: 1131.3.

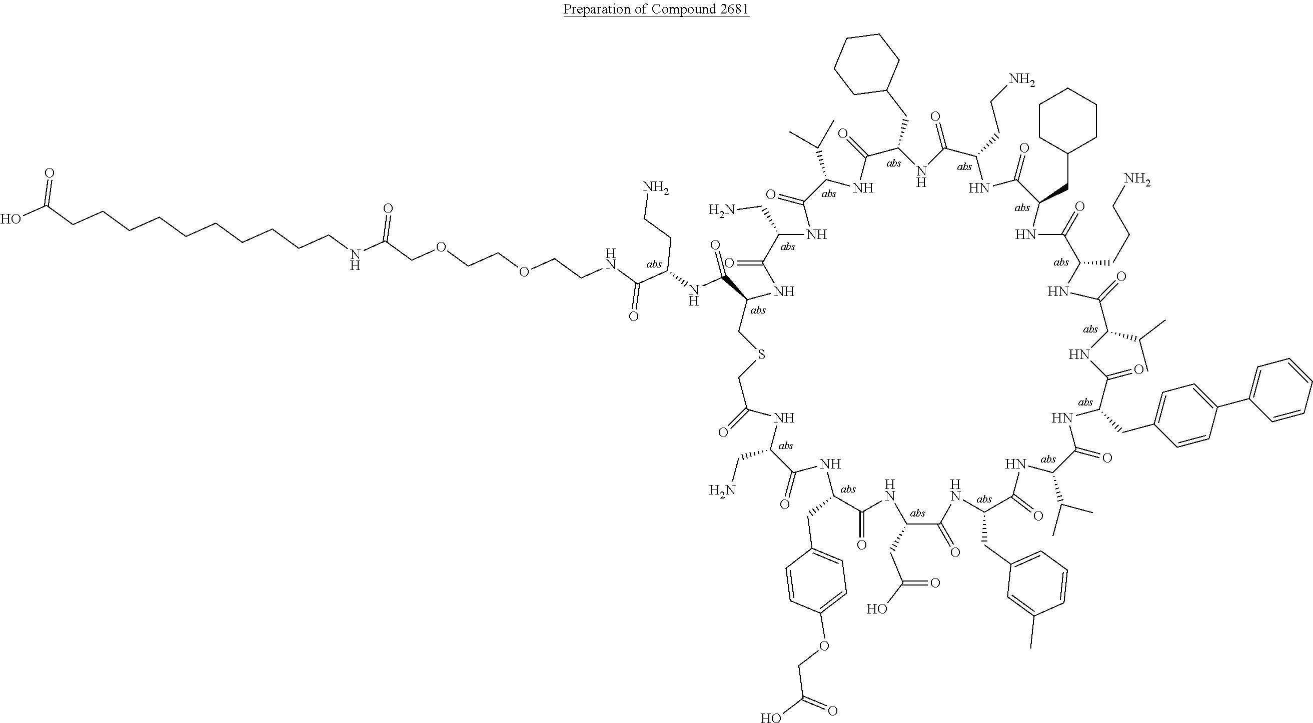 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC3CCCCC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2681 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12 mg, and its estimated purity by LCMS analysis was 98.6%. Analysis condition A: Retention time=2.31 min; ESI-MS(+) m/z [M+2H]²⁺: 1151.1.

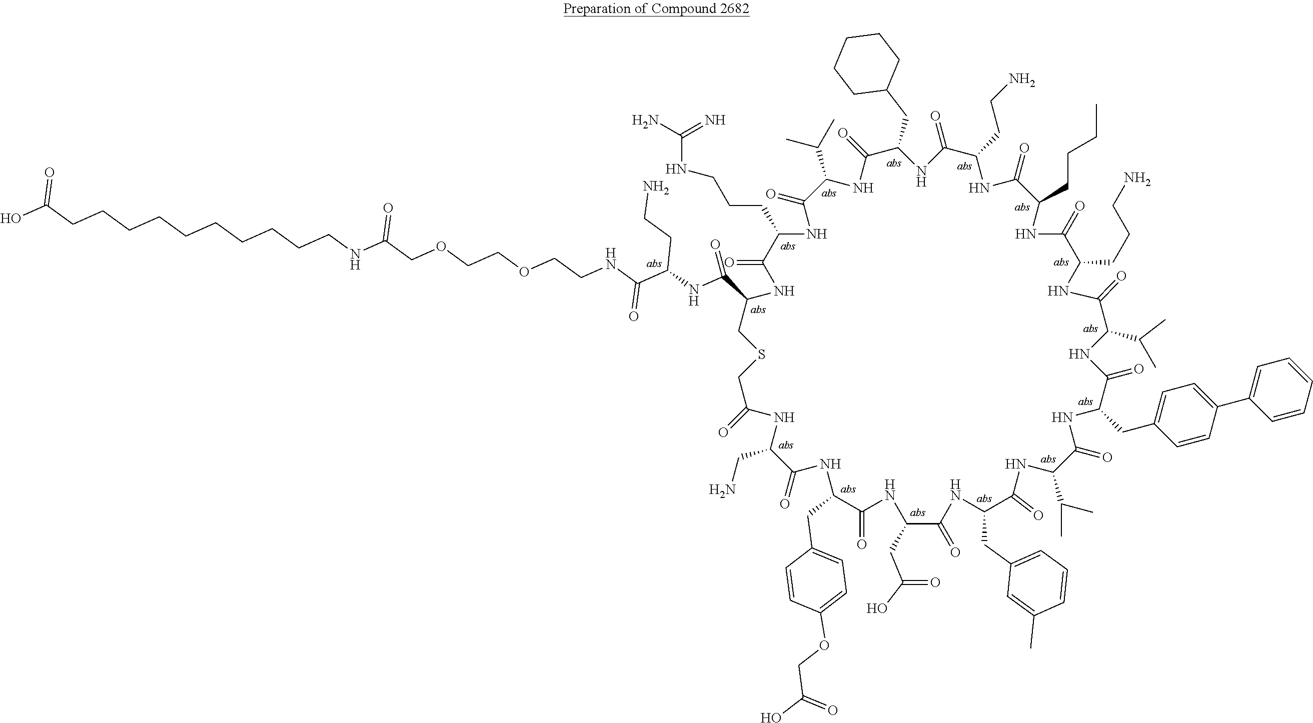 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 2682 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.5 mg, and its estimated purity by LCMS analysis was 94.8%. Analysis condition A: Retention time=1.93 min; ESI-MS(+) m/z [M+2H]²⁺: 1166.1.

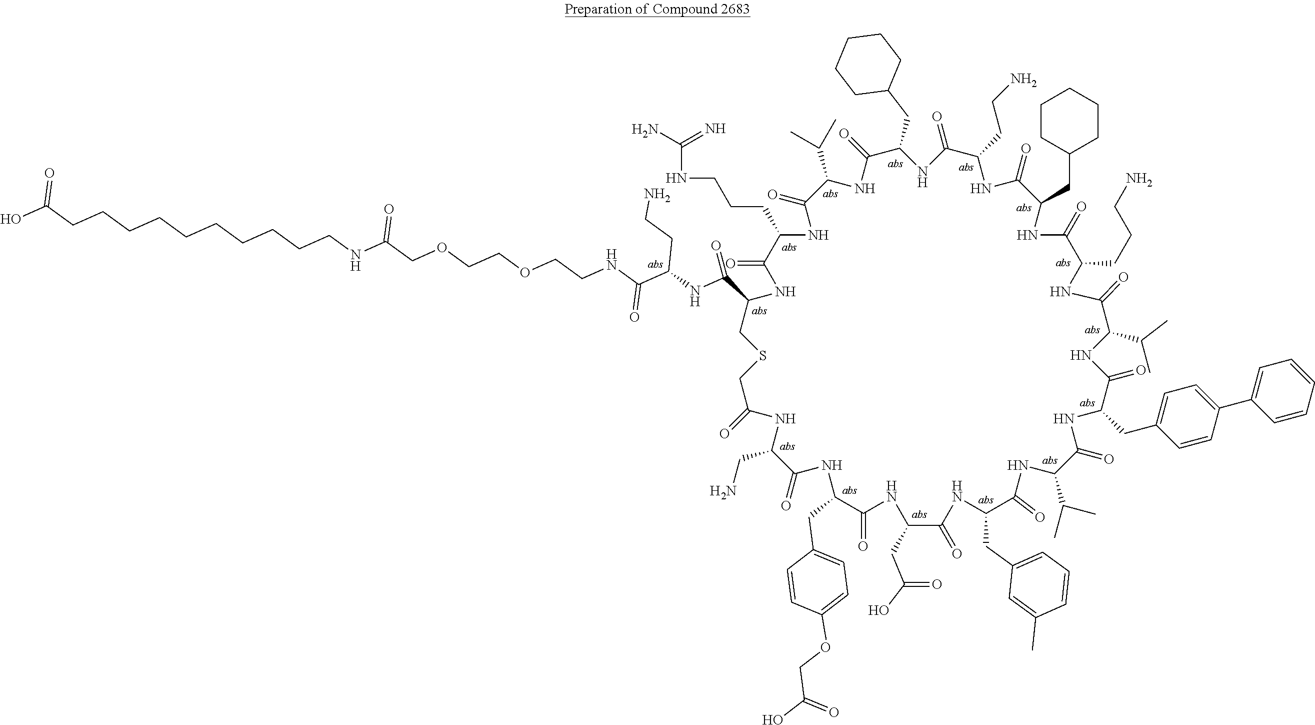 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC3CCCCC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2683 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.9 mg, and its estimated purity by LCMS analysis was 95.5%. Analysis condition B: Retention time=1.6 min; ESI-MS(+) m/z [M+3H]³⁺: 791.1.

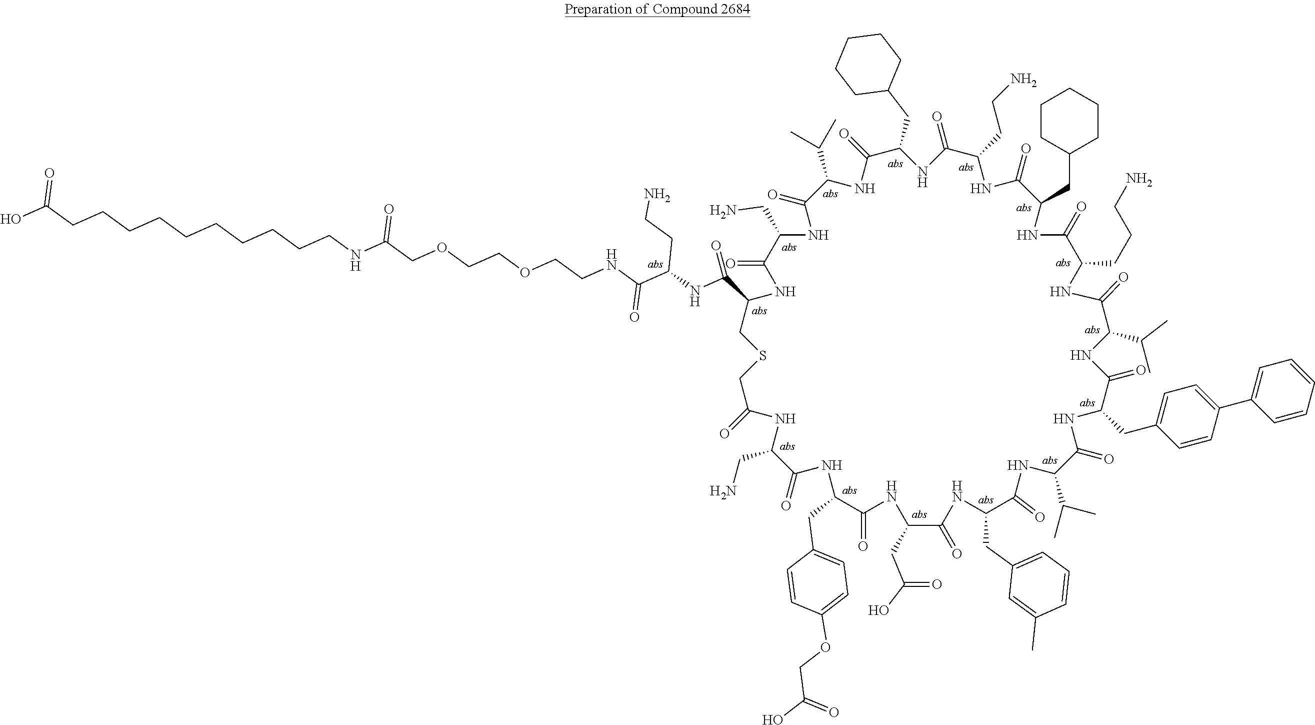 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC3CCCCC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2684 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.9 mg, and its estimated purity by LCMS analysis was 99%. Analysis condition A: Retention time=1.83 min; ESI-MS(+) m/z [M+2H]²⁺: 1151.1.

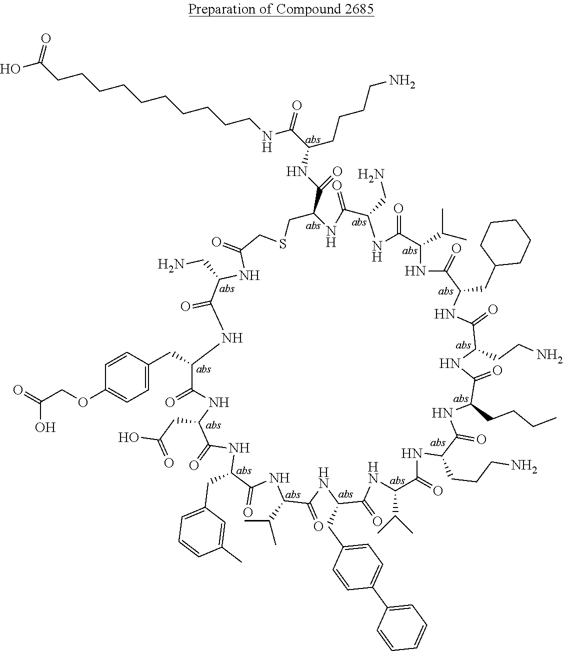 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 2685 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.7 mg, and its estimated purity by LCMS analysis was 93.2%. Analysis condition B: Retention time=1.56 min; ESI-MS(+) m/z [M+3H]³⁺: 715.4.

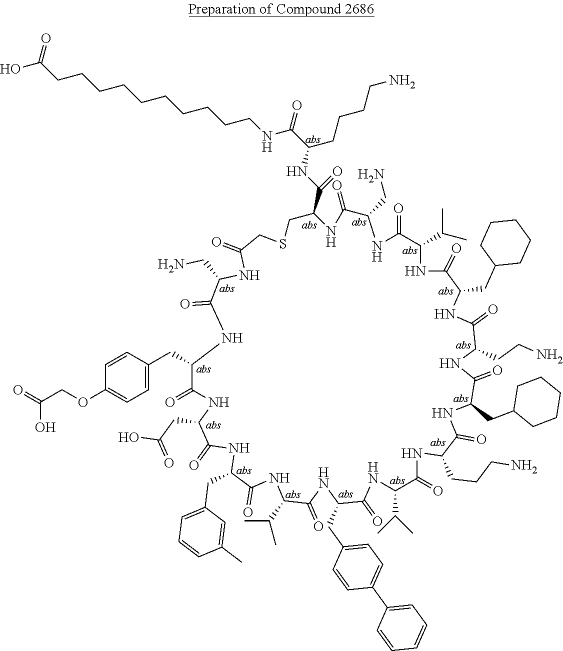 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC3CCCCC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2686 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.6 mg, and its estimated purity by LCMS analysis was 97.8%. Analysis condition B: Retention time=1.6 min; ESI-MS(+) m/z [M+3H]³⁺: 728.9.

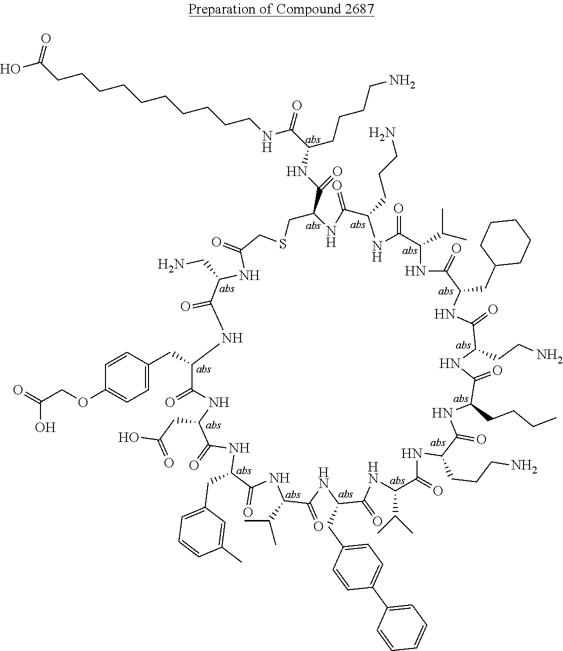 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2687 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.1 mg, and its estimated purity by LCMS analysis was 100%. Analysis condition A: Retention time=1.58 min; ESI-MS(+) m/z [M+3H]³⁺: 724.4.

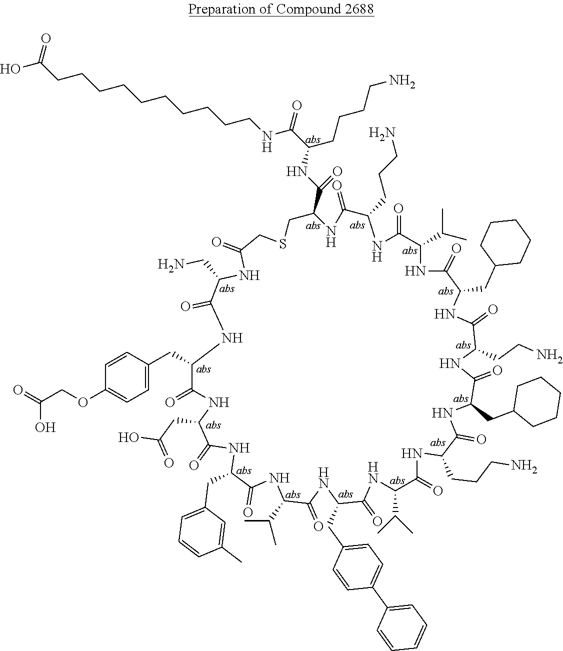 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC3CCCCC3)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2688 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.4 mg, and its estimated purity by LCMS analysis was 93%. Analysis condition B: Retention time=1.58 min; ESI-MS(+) m/z [M+2H]²⁺: 1106.4.

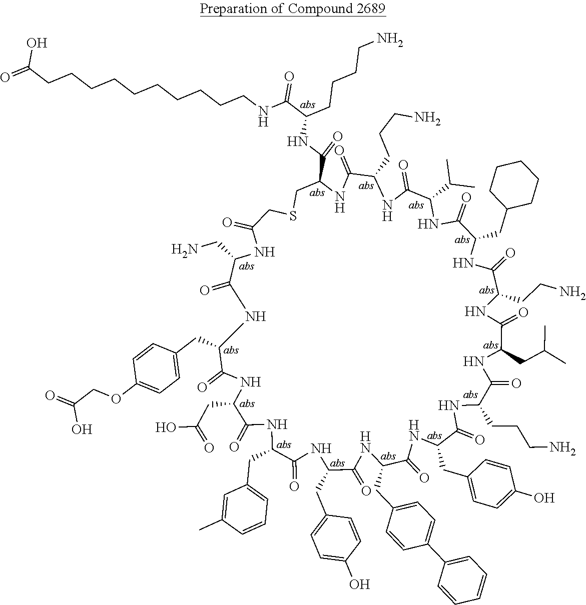 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2689 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered centrifugal evaporation. The yield of the product was 13.4 mg, and its estimated purity by LCMS analysis was 92.6%. Analysis condition B: Retention time=1.42 min; ESI-MS(+) m/z [M+3H]³⁺: 758.2.

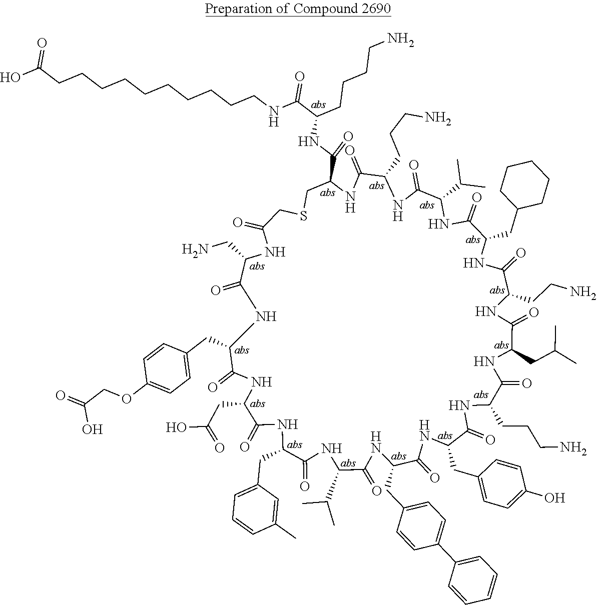 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2690 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 17.3 mg, and its estimated purity by LCMS analysis was 92.8%. Analysis condition B: Retention time=1.5 min; ESI-MS(+) m/z [M+2H]²⁺: 1105.2.

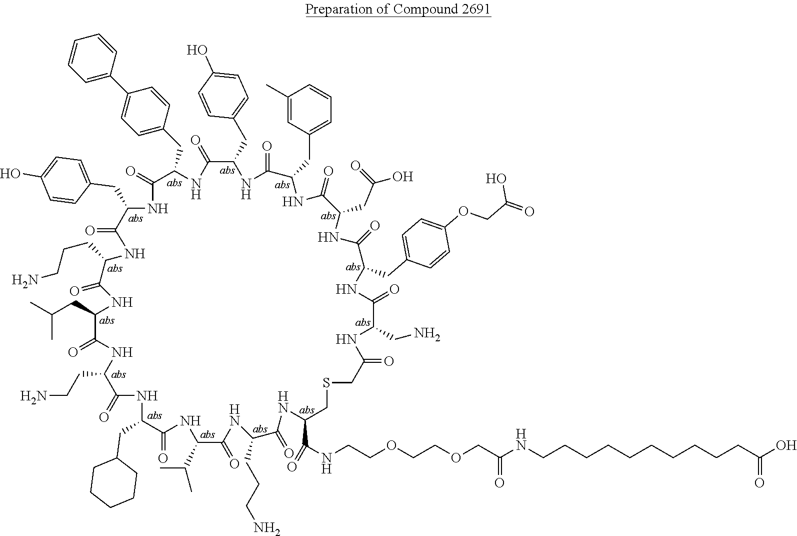 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2691 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 21.9 mg, and its estimated purity by LCMS analysis was 99.2%. Analysis condition A: Retention time=1.58 min; ESI-MS(+) m/z [M+2H]²⁺: 1159.3.

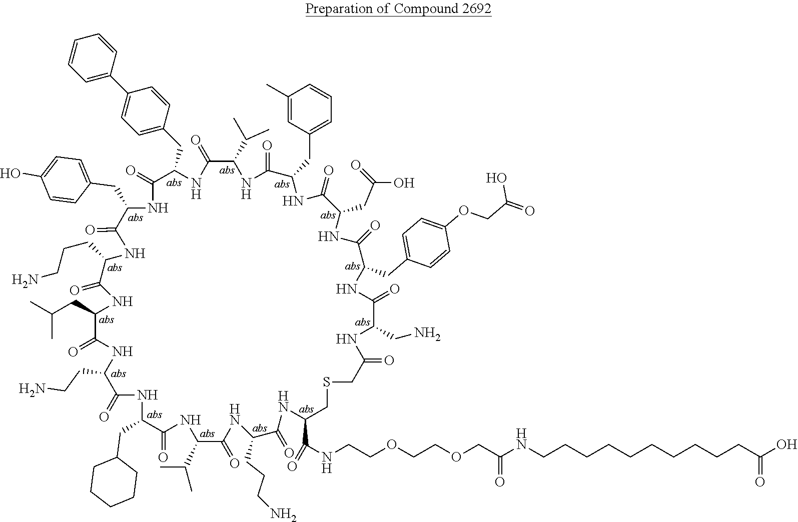 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2692 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 16.7 mg, and its estimated purity by LCMS analysis was 100%. Analysis condition A: Retention time=1.59 min; ESI-MS(+) m/z [M+2H]²⁺: 1127.1.

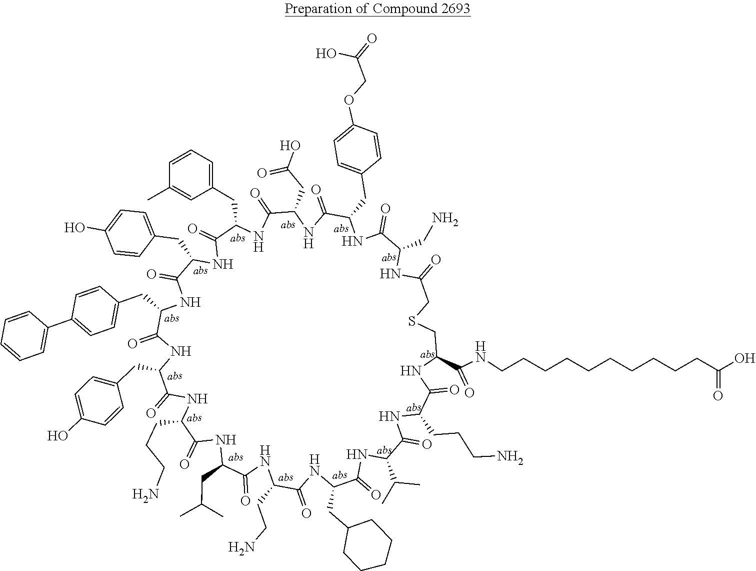 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2693 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.6 mg, and its estimated purity by LCMS analysis was 97.4%. Analysis condition A: Retention time=1.63 min; ESI-MS(+) m/z [M+3H]³⁺: 724.9.

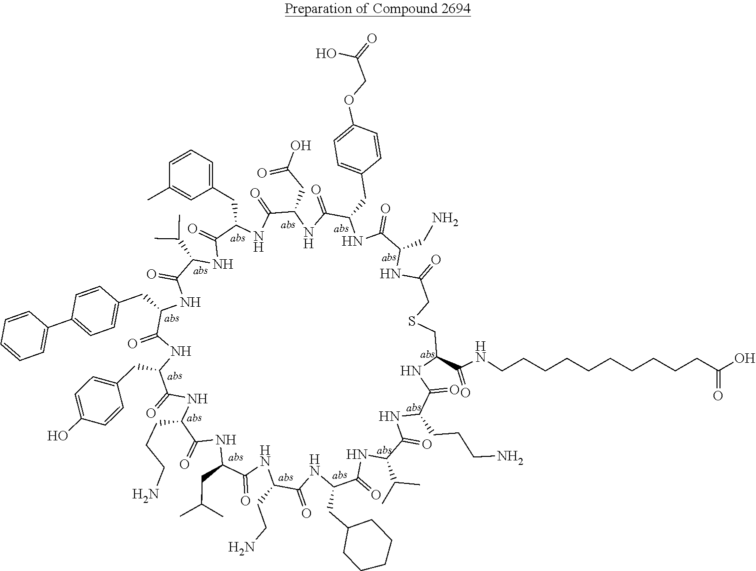 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2694 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 14.2 mg, and its estimated purity by LCMS analysis was 97.9%. Analysis condition A: Retention time=1.65 min; ESI-MS(+) m/z [M+2H]²⁺: 1054.2.

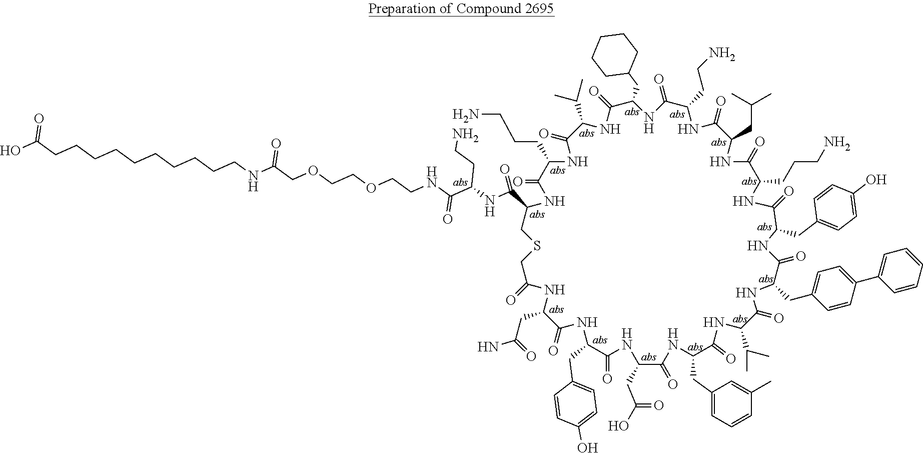 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2695 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.9 mg, and its estimated purity by LCMS analysis was 92.7%. Analysis condition A: Retention time=2.15 min; ESI-MS(+) m/z [M+3H]³⁺: 775.2.

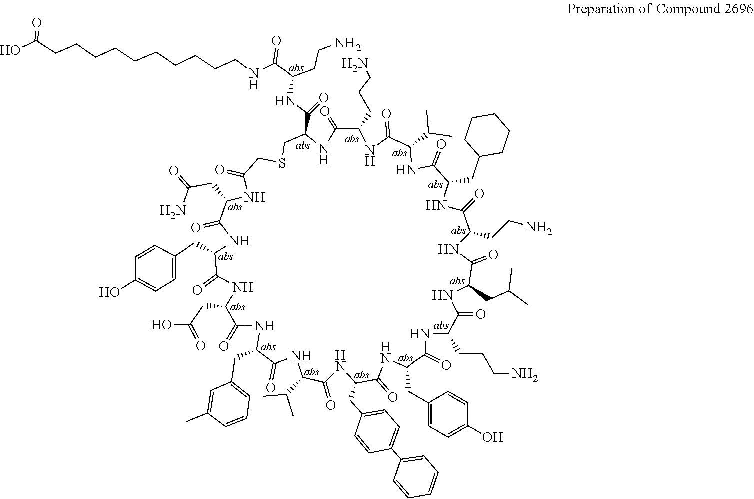 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCN)CC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](C(C)C)NC2=O)c1]

Compound 2696 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.1 mg, and its estimated purity by LCMS analysis was 95.4%. Analysis condition A: Retention time=2.16 min; ESI-MS(+) m/z [M+3H]³⁺: 726.9.

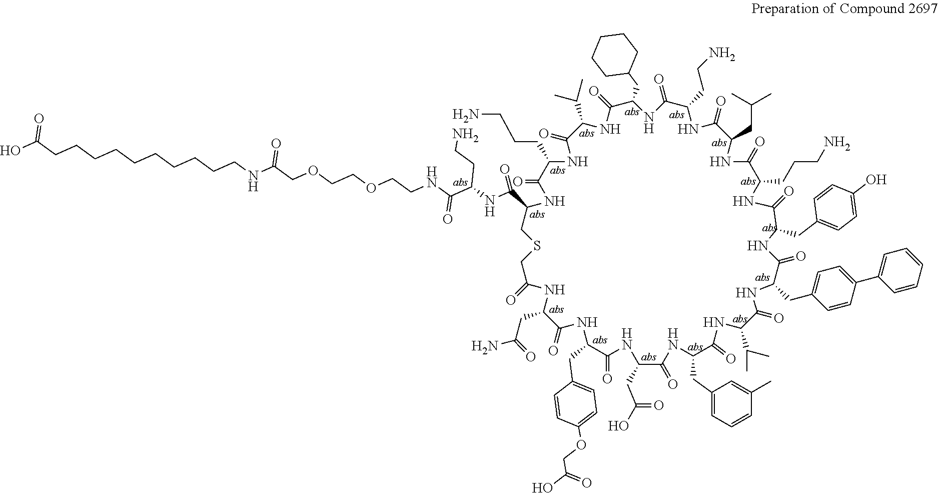 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2697 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.6 mg, and its estimated purity by LCMS analysis was 97.7%. Analysis condition B: Retention time=1.49 min; ESI-MS(+) m/z [M+2H]²⁺: 1191.1.

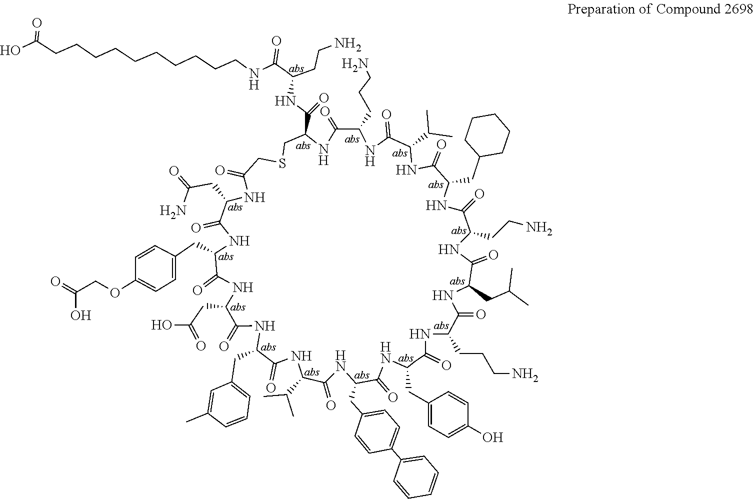 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)CC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](C(C)C)NC2=O)c1]

Compound 2698 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4.8 mg, and its estimated purity by LCMS analysis was 100%. Analysis condition A: Retention time=1.59 min; ESI-MS(+) m/z [M+3H]³⁺: 746.

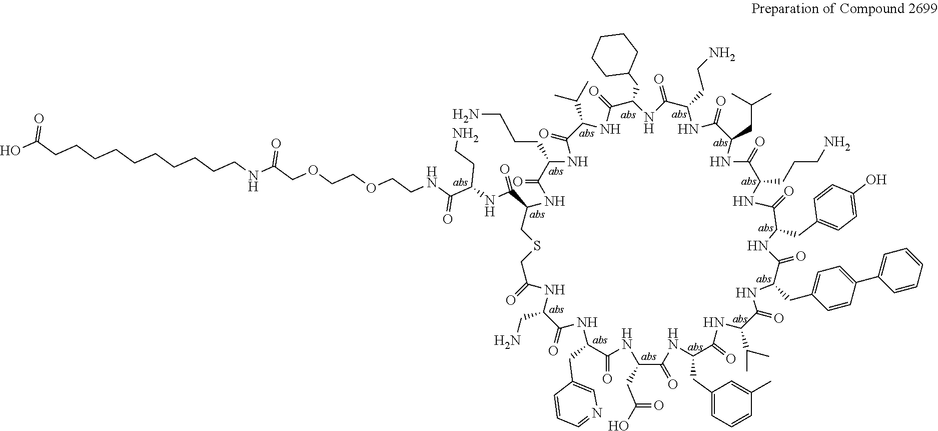 [SMILES: Cc1cccc(C[C@@H]2NC(=O)[C@H](CC(=O)O)NC(=O)[C@H](Cc3cccnc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2699 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 12.7 mg, and its estimated purity by LCMS analysis was 100%. Analysis condition A: Retention time=1.64 min; ESI-MS(+) m/z [M+3H]³⁺: 770.1.

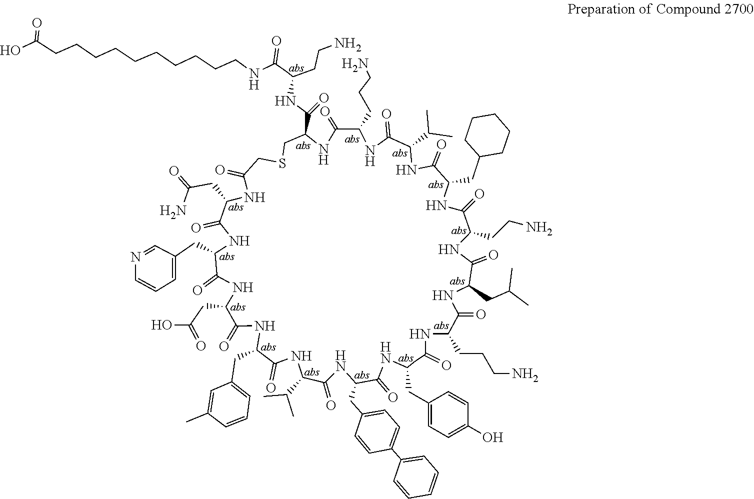 [SMILES: Cc1cccc(C[C@@H]2NC(=O)[C@H](CC(=O)O)NC(=O)[C@H](Cc3cccnc3)NC(=O)[C@H](CC(N)=O)NC(=O)CSC[C@@H](C(=O)N[C@@H](CCN)CC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](C(C)C)NC2=O)c1]

Compound 2700 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 9% B, 9-49% B over 20 minutes, then a 2-minute hold at centrifugal evaporation. The yield of the product was 17.1 mg, and its estimated purity by LCMS analysis was 97.8%. Analysis condition A: Retention time=1.58 min; ESI-MS(+) m/z [M+2H]²⁺: 1082.2.

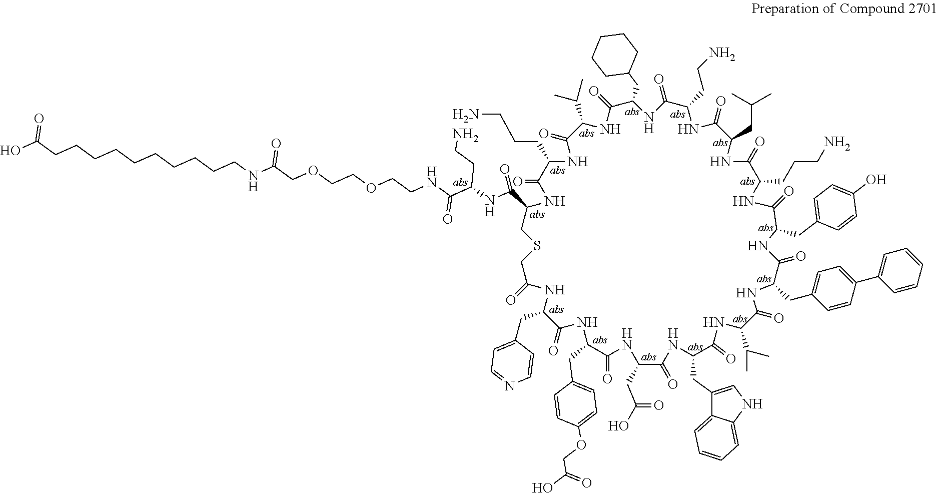 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2701 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.1 mg, and its estimated purity by LCMS analysis was 98.4%. Analysis condition A: Retention time=1.66 min; ESI-MS(+) m/z [M+3H]³⁺: 814.1.

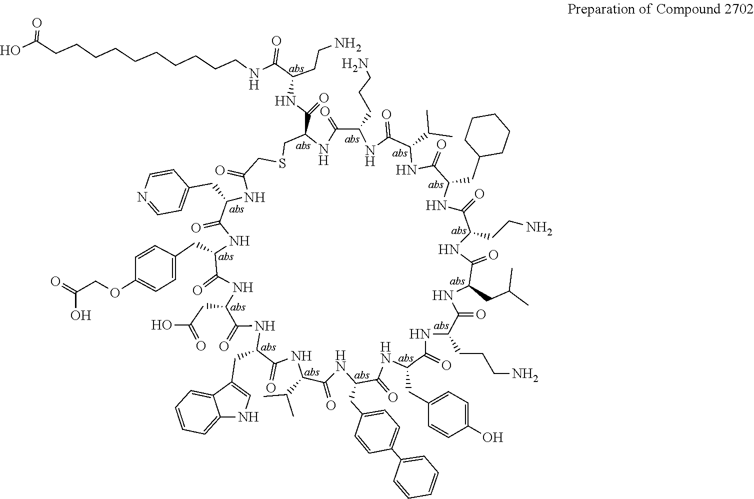 [SMILES: CC(C)C[C@H]1NC(=O)[C@H](CCCN)NC(=O)C[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)CC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2702 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-50% B over 20 minutes, then a 2-minute hold at centrifugal evaporation. The yield of the product was 4.8 mg, and its estimated purity by LCMS analysis was 100%. Analysis condition A: Retention time=1.54 min; ESI-MS(+) m/z [M+2H]²⁺: 1147.9.

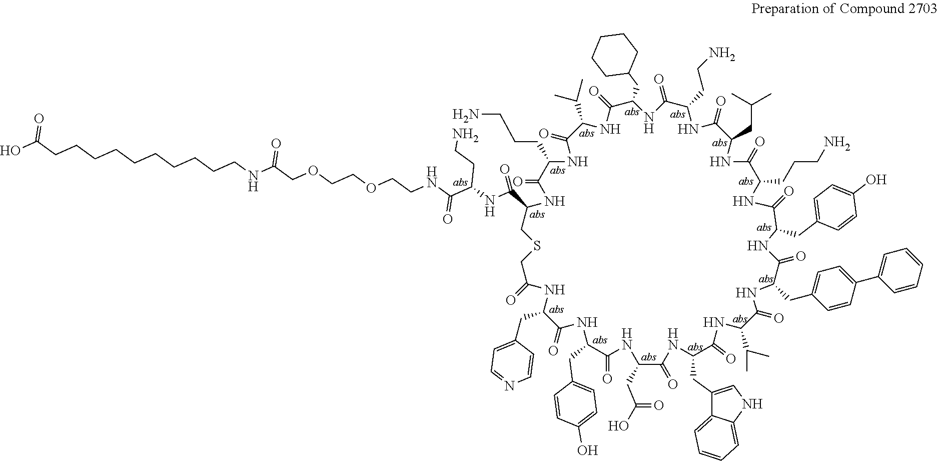 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2703 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 22 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.6 mg, and its estimated purity by LCMS analysis was 95.7%. Analysis condition A: Retention time=2.05 min; ESI-MS(+) m/z [M+3H]³⁺: 795.1.

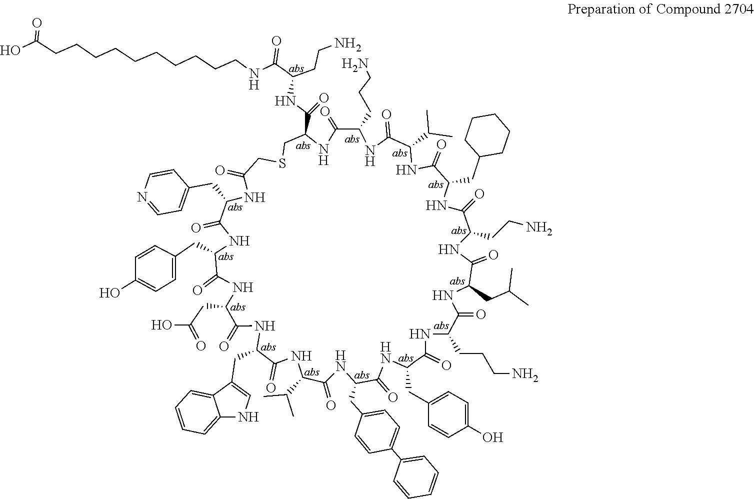 [SMILES: CC(C)C[C@H]1NC(=O)[C@H](CCCN)NC(=O)C[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CCN)CC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 2704 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.6 mg, and its estimated purity by LCMS analysis was 93.1%. Analysis condition B: Retention time=1.36 min; ESI-MS(+) m/z [M+3H]³⁺: 746.

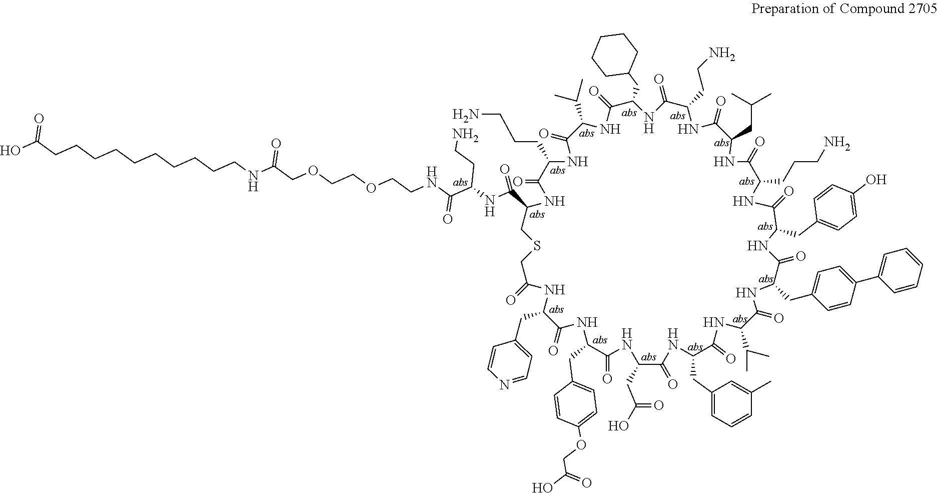 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2705 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.9 mg, and its estimated purity by LCMS analysis was 95.5%. Analysis condition A: Retention time=1.57 min; ESI-MS(+) m/z [M+2H]²⁺: 1208.1.

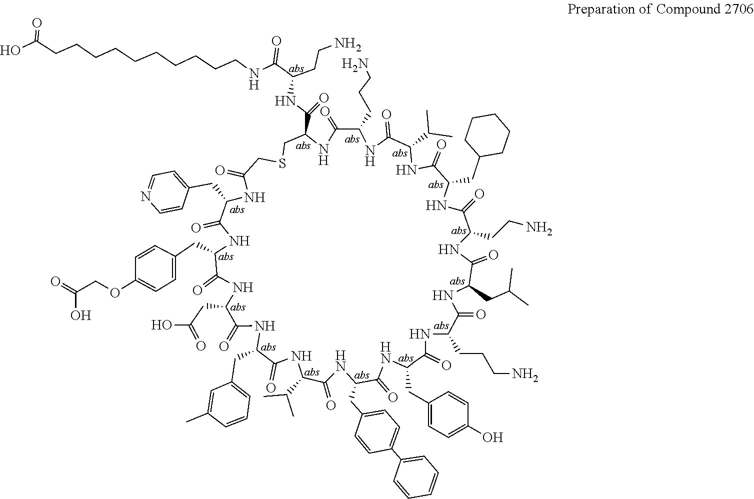 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)CC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](C(C)C)NC2=O)c1]

Compound 2706 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-50% B over 20 minutes, then a 2-minute hold at centrifugal evaporation. The yield of the product was 5.8 mg, and its estimated purity by LCMS analysis was 97.8%. Analysis condition B: Retention time=1.43 min; ESI-MS(+) m/z [M+3H]³⁺: 757.5.

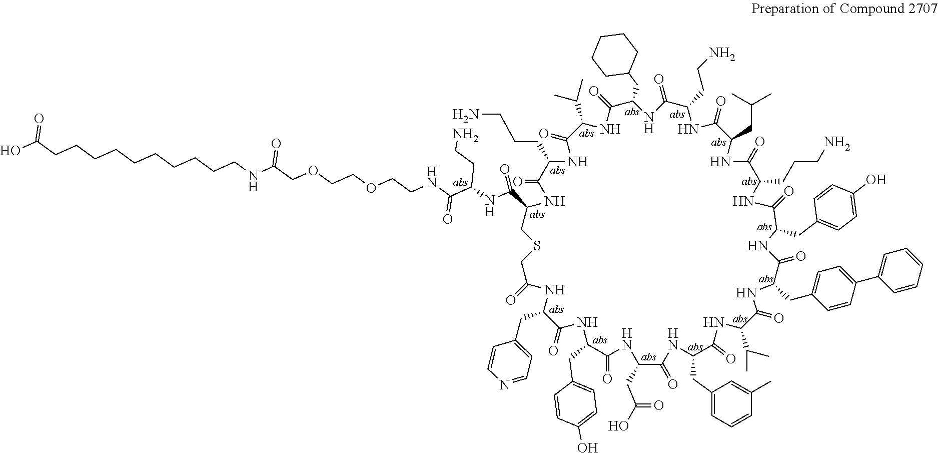 [SMILES: Cc1cccc(C[C@@H]2NC(=O)[C@H](CC(=O)O)NC(=O)[C@H](Cc3ccc(O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2707 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-50% B over 20 minutes, then a 2-minute hold at centrifugal evaporation. The yield of the product was 26.9 mg, and its estimated purity by LCMS analysis was 100%. Analysis condition A: Retention time=1.65 min; ESI-MS(+) m/z [M+2H]²⁺: 1178.9.

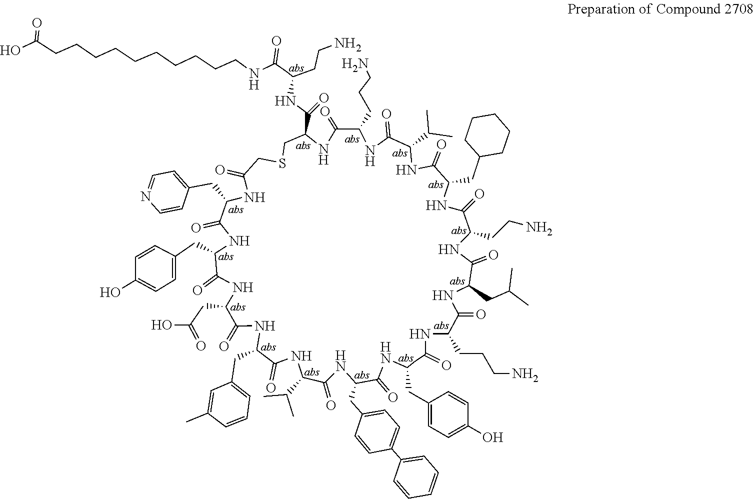 [SMILES: Cc1cccc(C[C@@H]2NC(=O)[C@H](CC(=O)O)NC(=O)[C@H](Cc3ccc(O)cc3)NC(=O)[C@H](Cc3ccncc3)NC(=O)CSC[C@@H](C(=O)N[C@@H](CCN)CC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](C(C)C)NC2=O)c1]

Compound 2708 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-50% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 16.8 mg, and its estimated purity by LCMS analysis was 100%. Analysis condition A: Retention time=1.66 min; ESI-MS(+) m/z [M+3H]³⁺: 738.1.

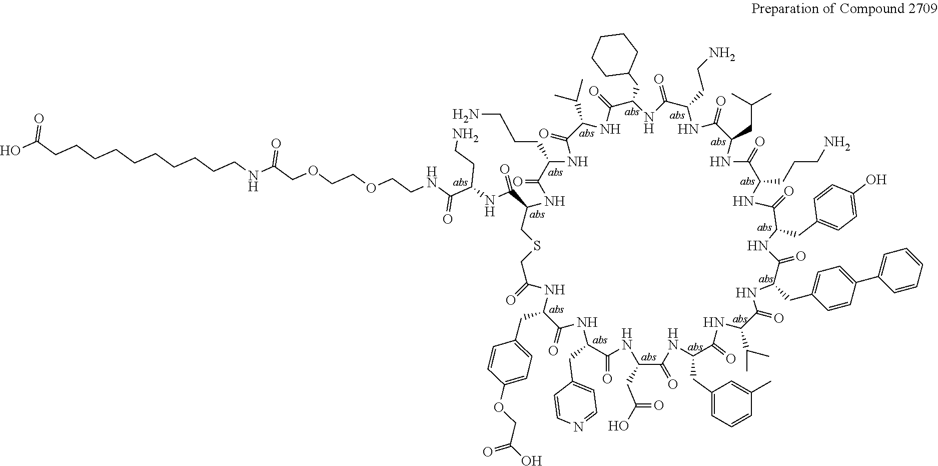 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2709 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20.7 mg, and its estimated purity by LCMS analysis was 90.5%. Analysis condition B: Retention time=1.41 min; ESI-MS(+) m/z [M+2H]²⁺: 1208.

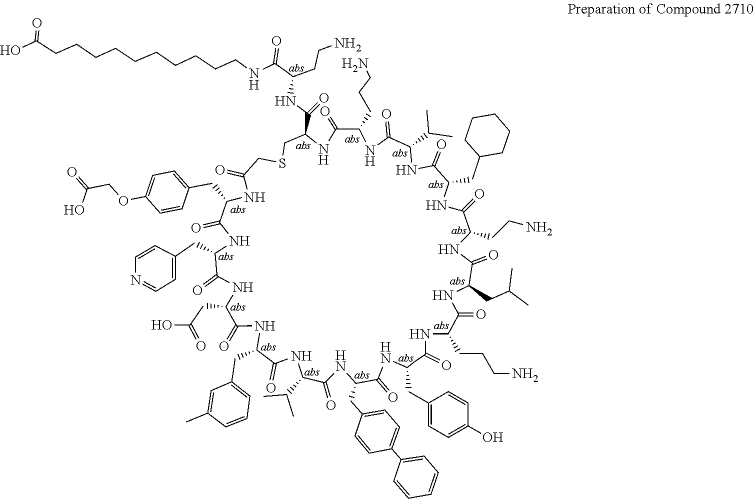 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)CC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](C(C)C)NC2=O)c1]

Compound 2710 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 9.1 mg, and its estimated purity by LCMS analysis was 96%. Analysis condition A: Retention time=1.8 min; ESI-MS(+) m/z [M+2H]²⁺: 1136.1.

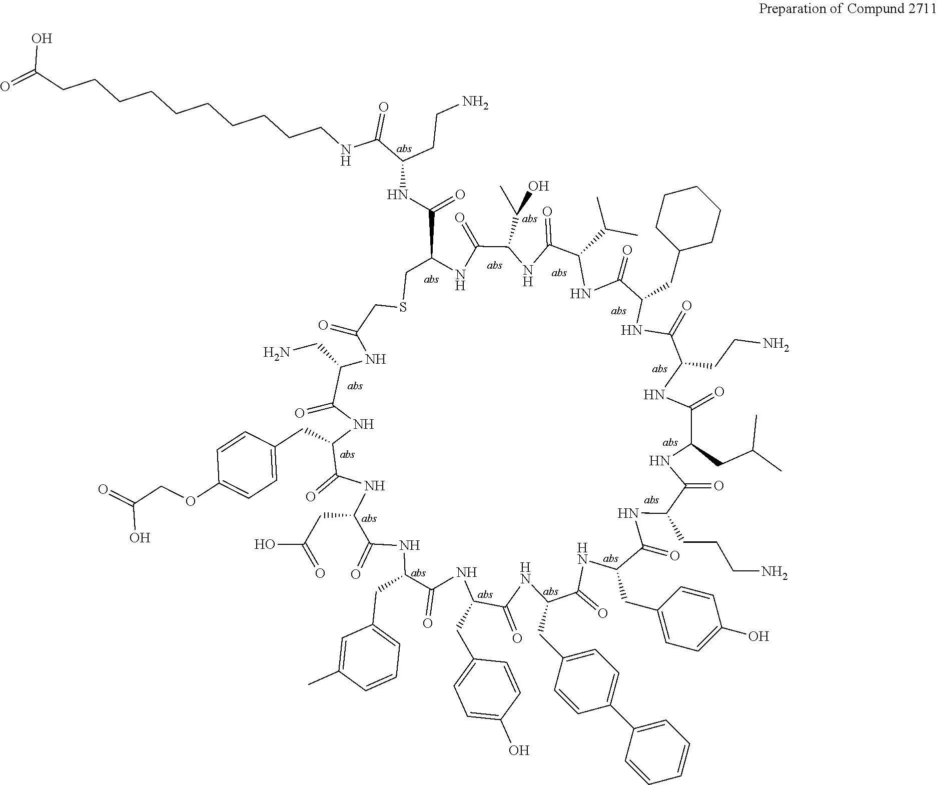 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2711 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 20.2 mg, and its estimated purity by LCMS analysis was 93.5%. Analysis condition B: Retention time=1.61 min; ESI-MS(+) m/z [M+2H]²⁺: 1130.3.

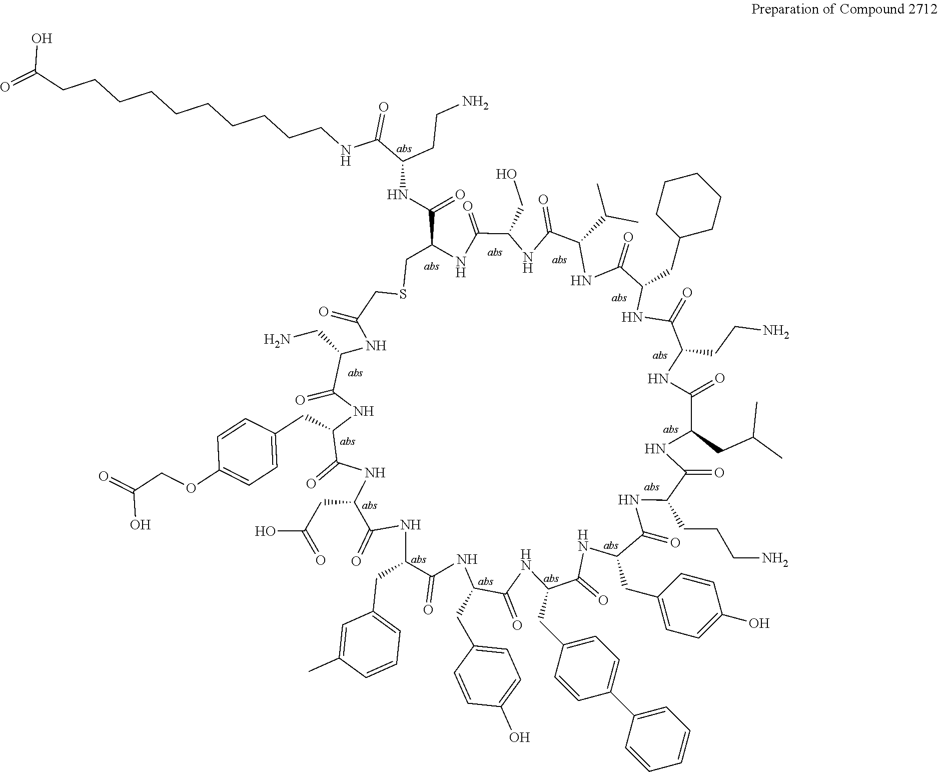 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2712 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.7 mg, and its estimated purity by LCMS analysis was 96.7%. Analysis condition A: Retention time=1.68 min; ESI-MS(+) m/z [M+2H]²⁺: 1123.2.

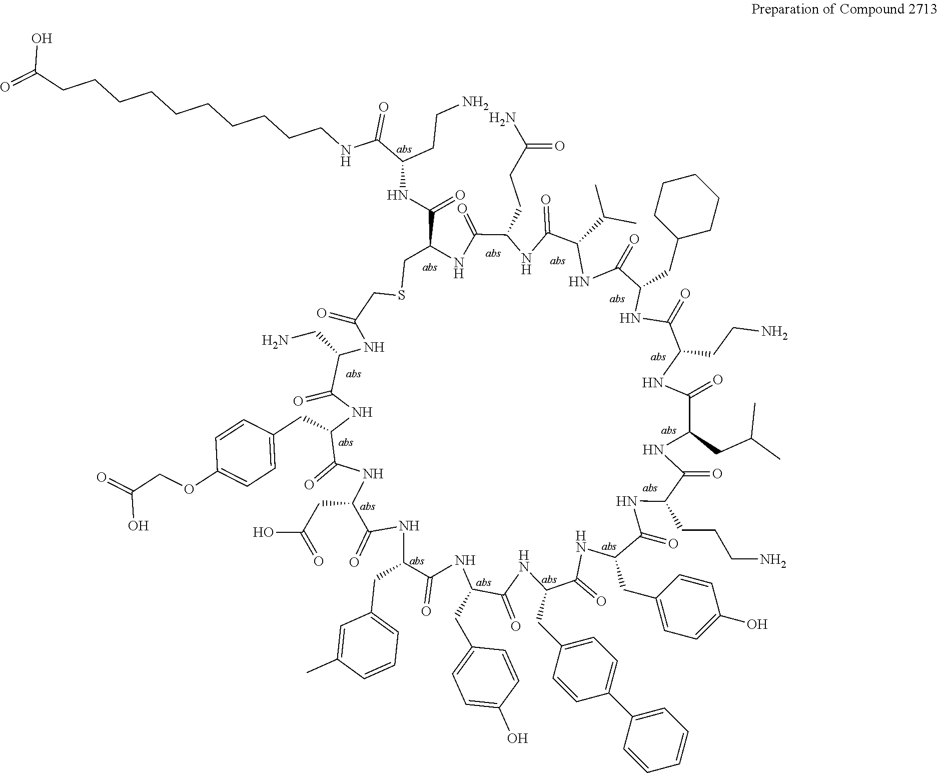 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2713 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-52% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 19.3 mg, and its estimated purity by LCMS analysis was 100%. Analysis condition A: Retention time=1.65 min; ESI-MS(+) m/z [M+3H]³⁺: 762.9.

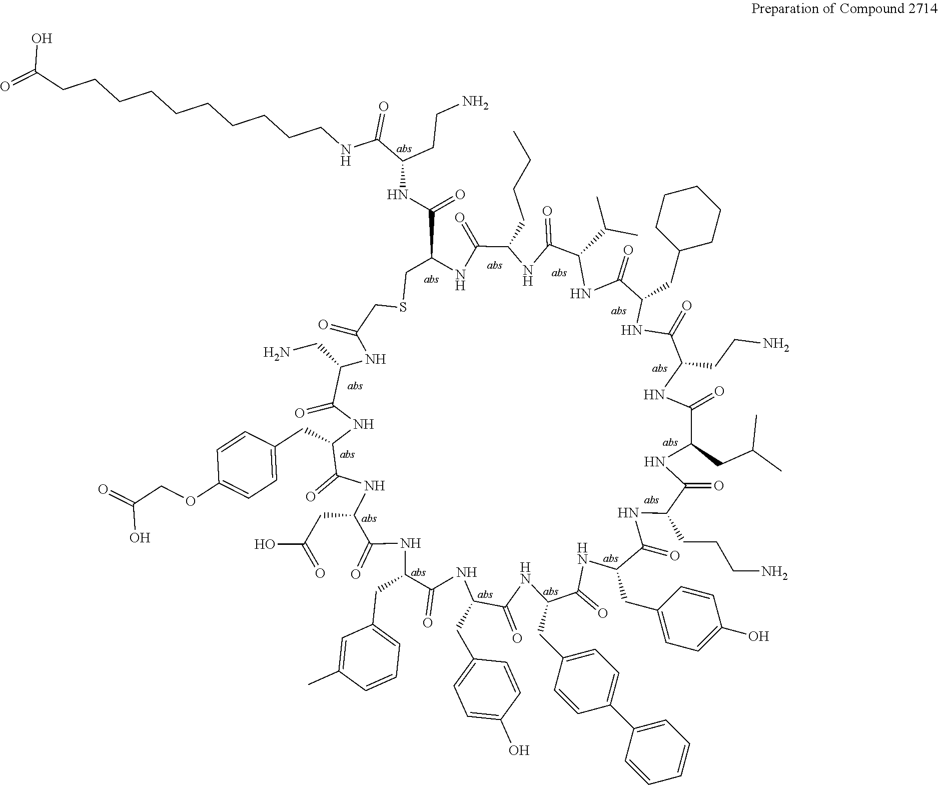 [SMILES: CCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC1=O]

Compound 2714 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 27% B, 27-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.3 mg, and its estimated purity by LCMS analysis was 85.8%. Analysis condition A: Retention time=1.78 min; ESI-MS(+) m/z [M+2H]²⁺: 1136.3.

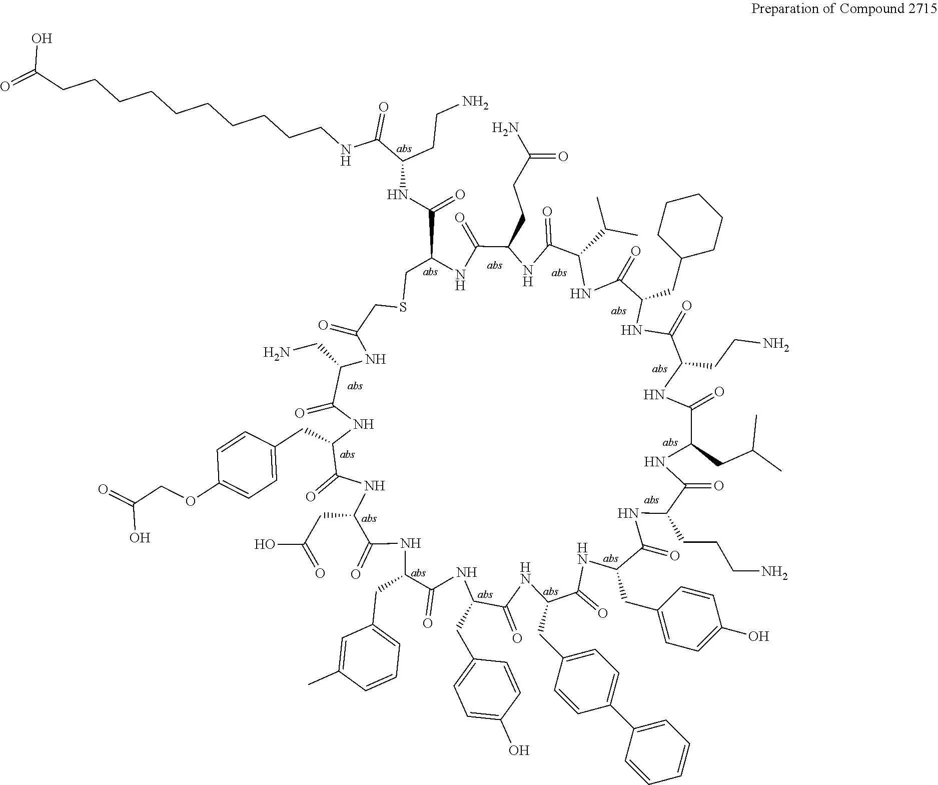 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2715 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 89.2 mg, and its estimated purity by LCMS analysis was 94.7%. Analysis condition A: Retention time=1.63 min; ESI-MS(+) m/z [M+3H]³⁺: 763.

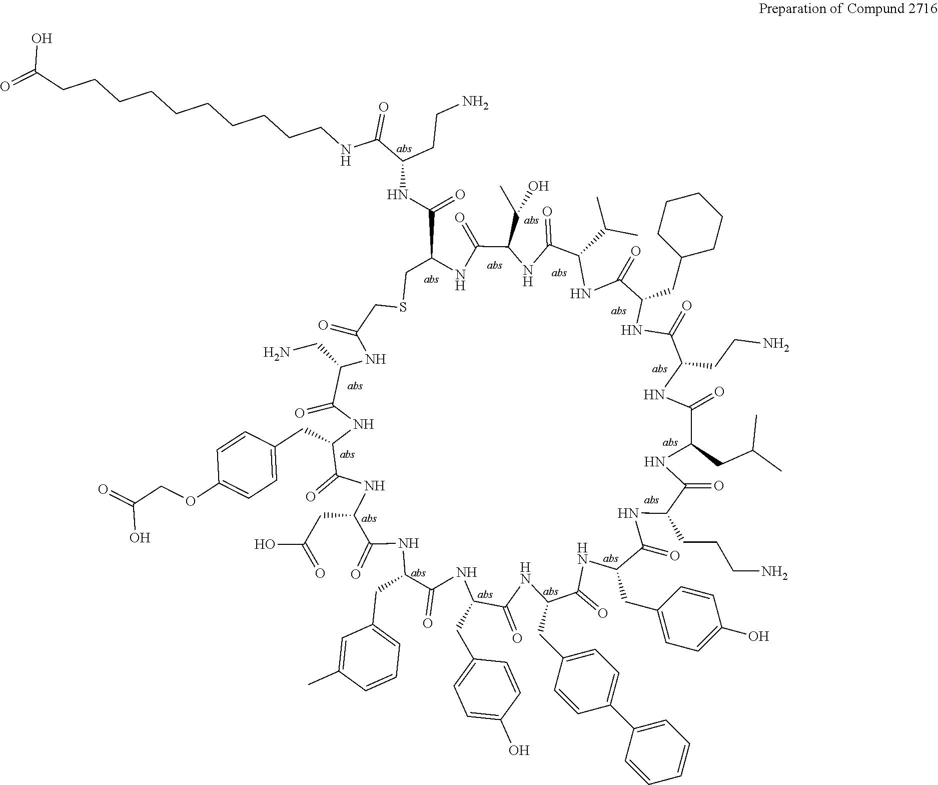 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H]([C@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2716 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 92.9 mg, and its estimated purity by LCMS analysis was 94%. Analysis condition B: Retention time=1.56 min; ESI-MS(+) m/z [M+2H]²⁺: 1130.1.

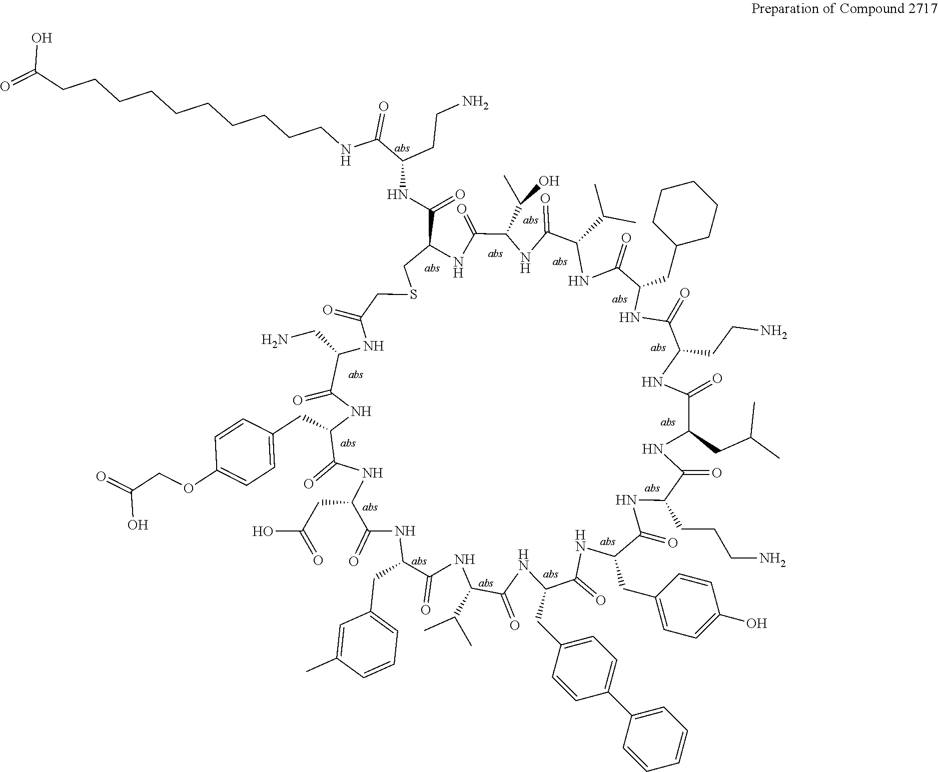 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2717 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.5 mg, and its estimated purity by LCMS analysis was 91.7%. Analysis condition B: Retention time=1.42 min; ESI-MS(+) m/z [M+2H]²⁺: 1098.1.

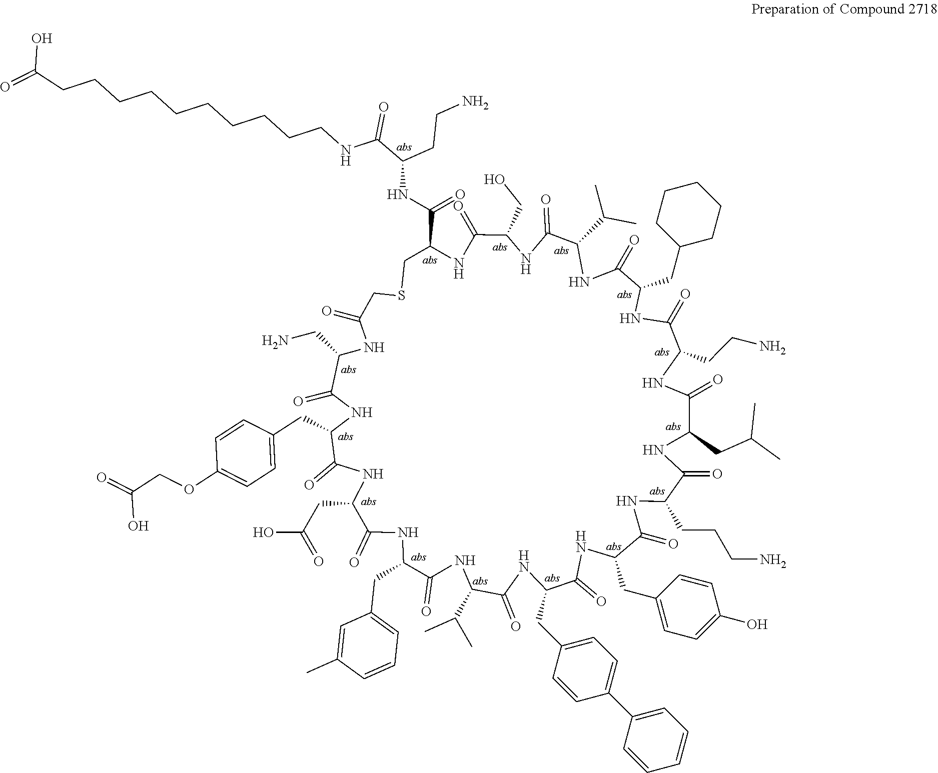 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2718 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.3 mg, and its estimated purity by LCMS analysis was 90%. Analysis condition B: Retention time=1.57 min; ESI-MS(+) m/z [M+2H]²⁺: 1090.9.

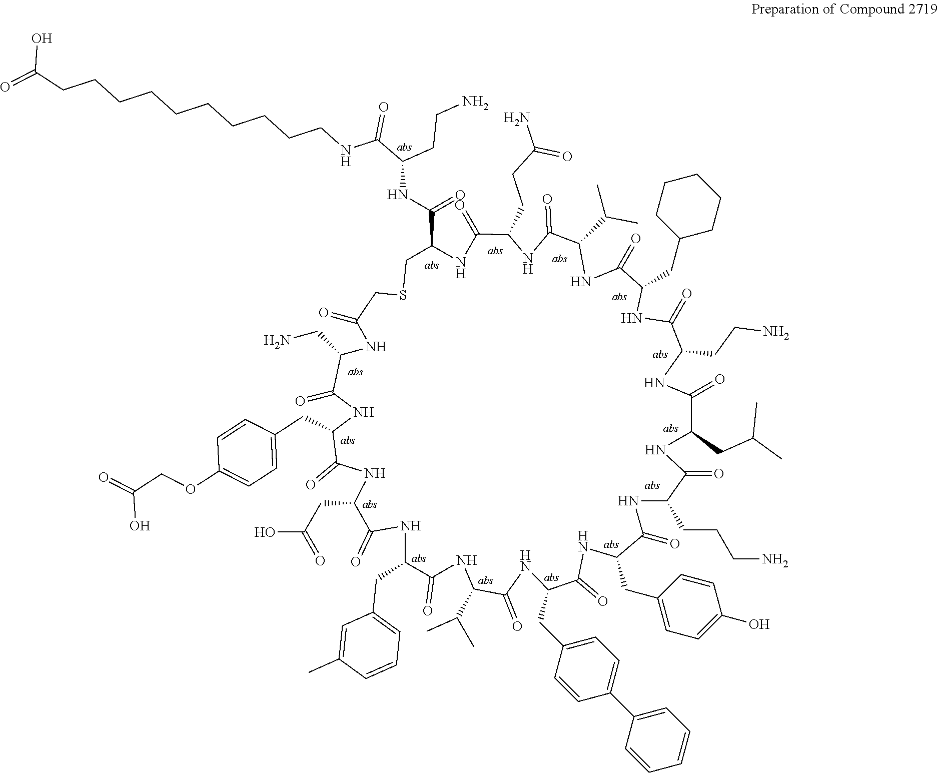 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2719 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 26 mg, and its estimated purity by LCMS analysis was 91.1%. Analysis condition A: Retention time=1.61 min; ESI-MS(+) m/z [M+3H]³⁺: 741.3.

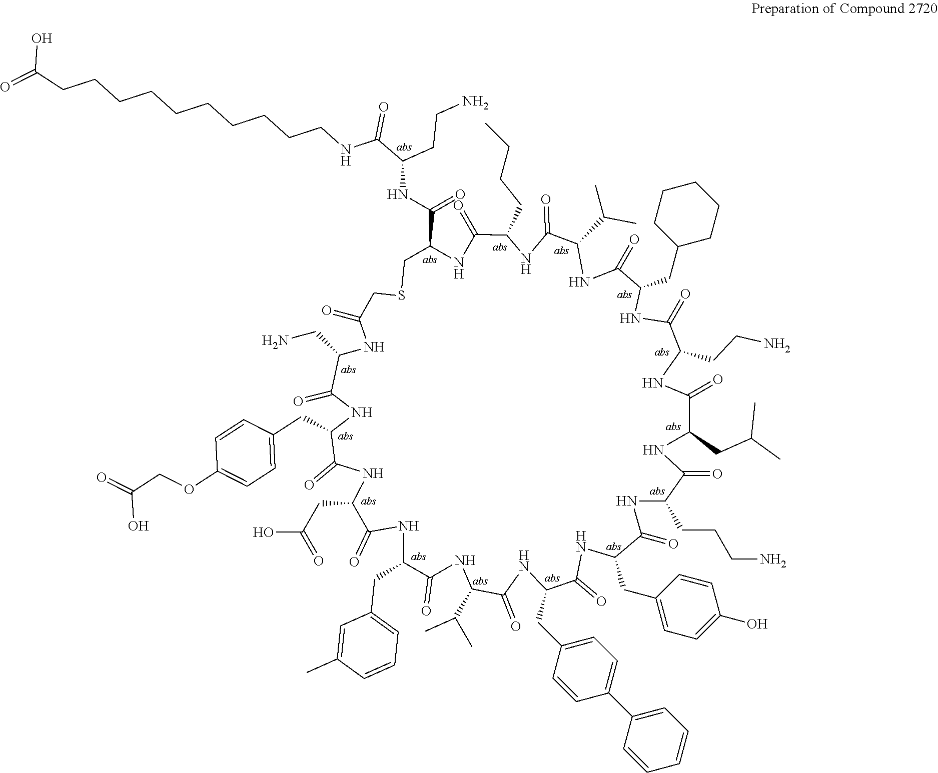 [SMILES: CCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC1=O]

Compound 2720 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.5 mg, and its estimated purity by LCMS analysis was 100%. Analysis condition A: Retention time=1.73 min; ESI-MS(+) m/z [M+2H]²⁺: 1104.2.

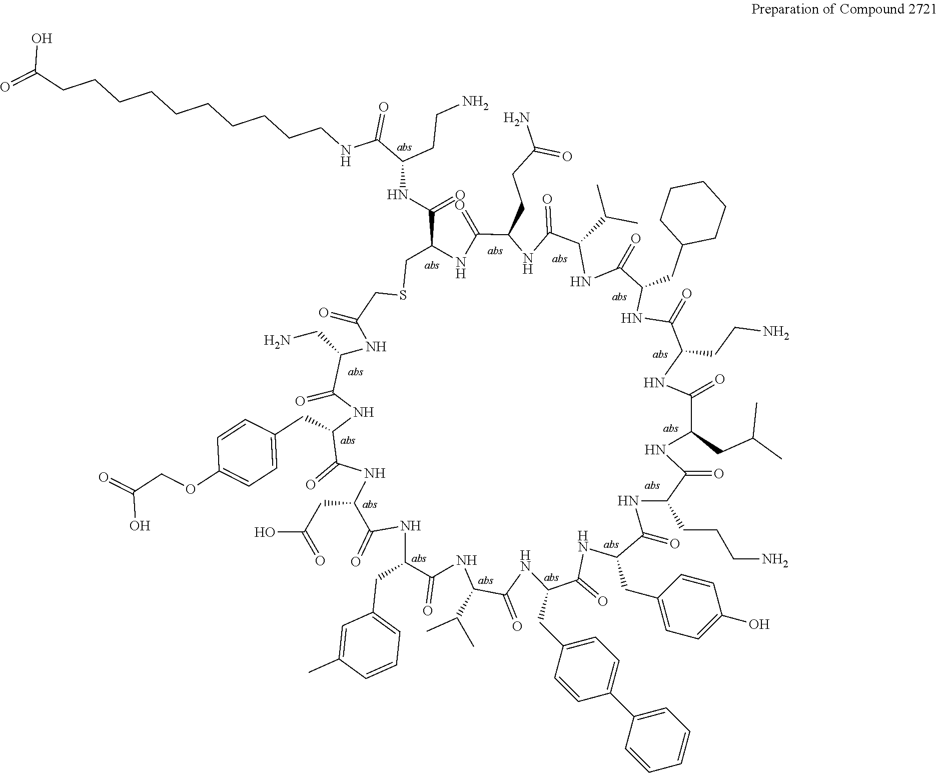 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2721 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 77.3 mg, and its estimated purity by LCMS analysis was 98.6%. Analysis condition A: Retention time=1.63 min; ESI-MS(+) m/z [M+2H]²⁺: 1111.9.

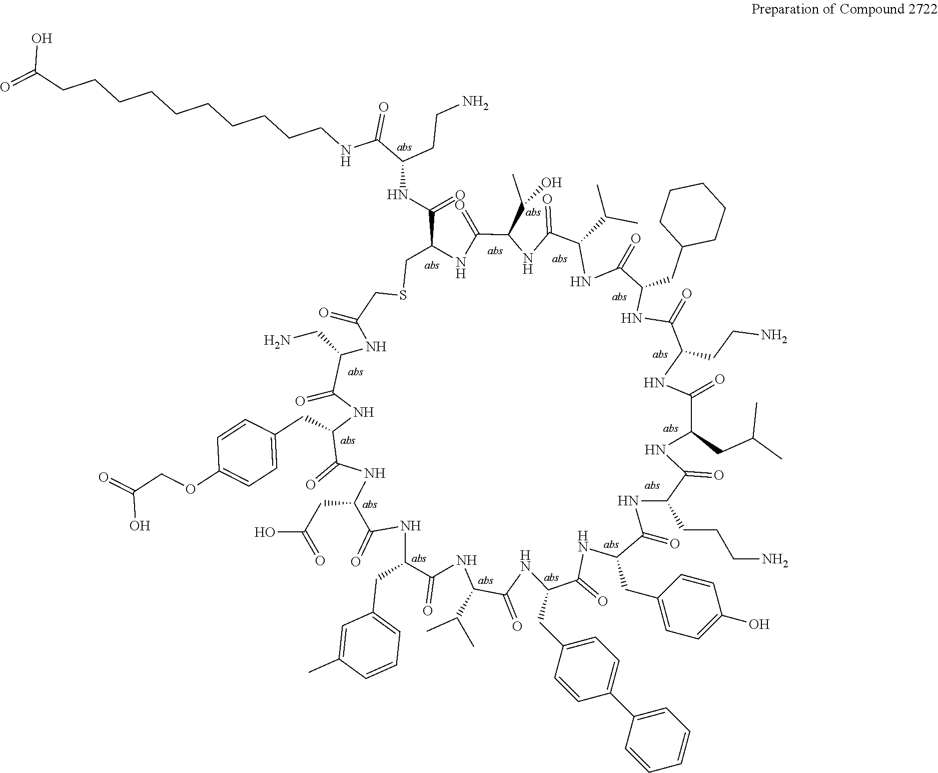 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H]([C@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2722 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 25 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 58 mg, and its estimated purity by LCMS analysis was 98.1%. Analysis condition A: Retention time=1.54 min; ESI-MS(+) m/z [M+2H]²⁺: 1098.1.

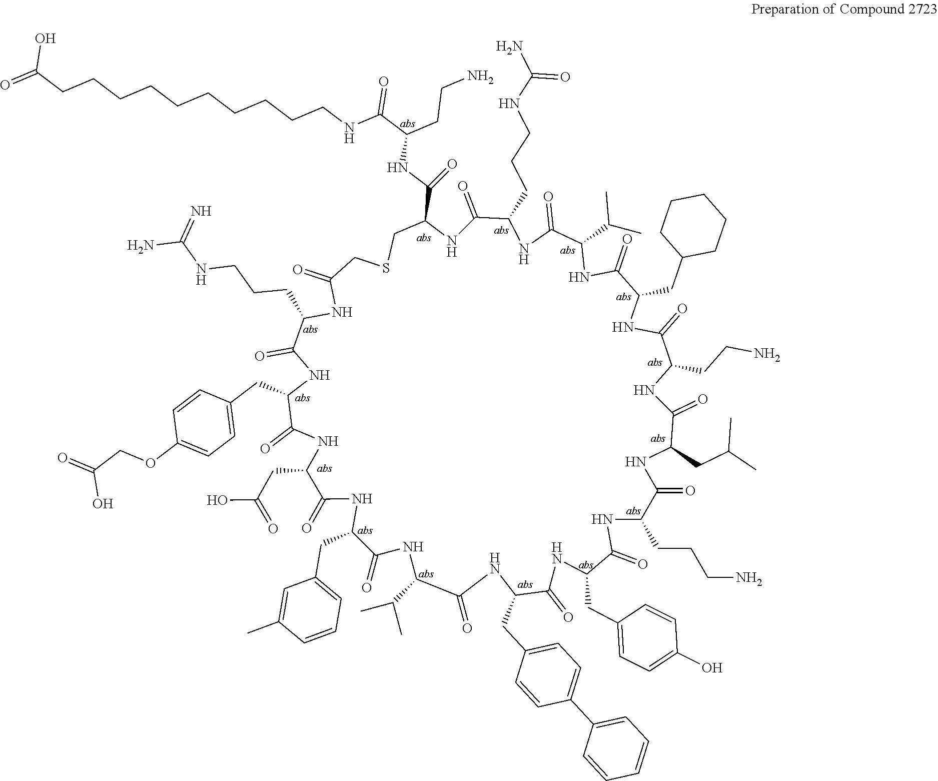 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2723 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.9 mg, and its estimated purity by LCMS analysis was 100%. Analysis condition A: Retention time=1.52 min; ESI-MS(+) m/z [M+2H]²⁺: 1161.1.

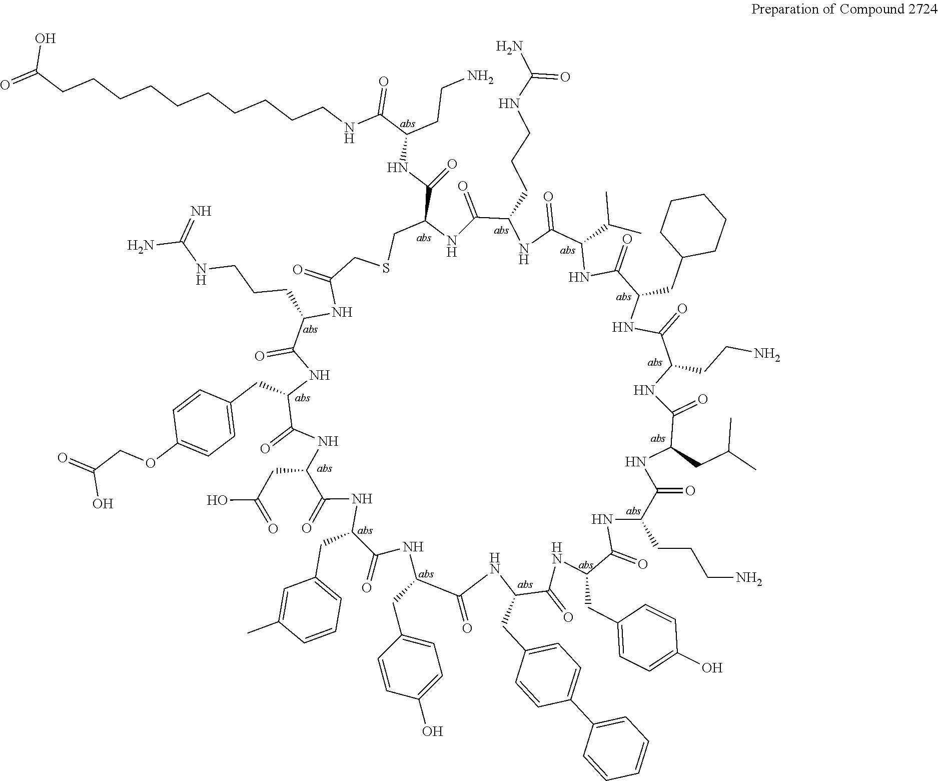 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2724 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.4 mg, and its estimated purity by LCMS analysis was 93.2%. Analysis condition A: Retention time=1.53 min; ESI-MS(+) m/z [M+2H]²⁺: 1193.6.

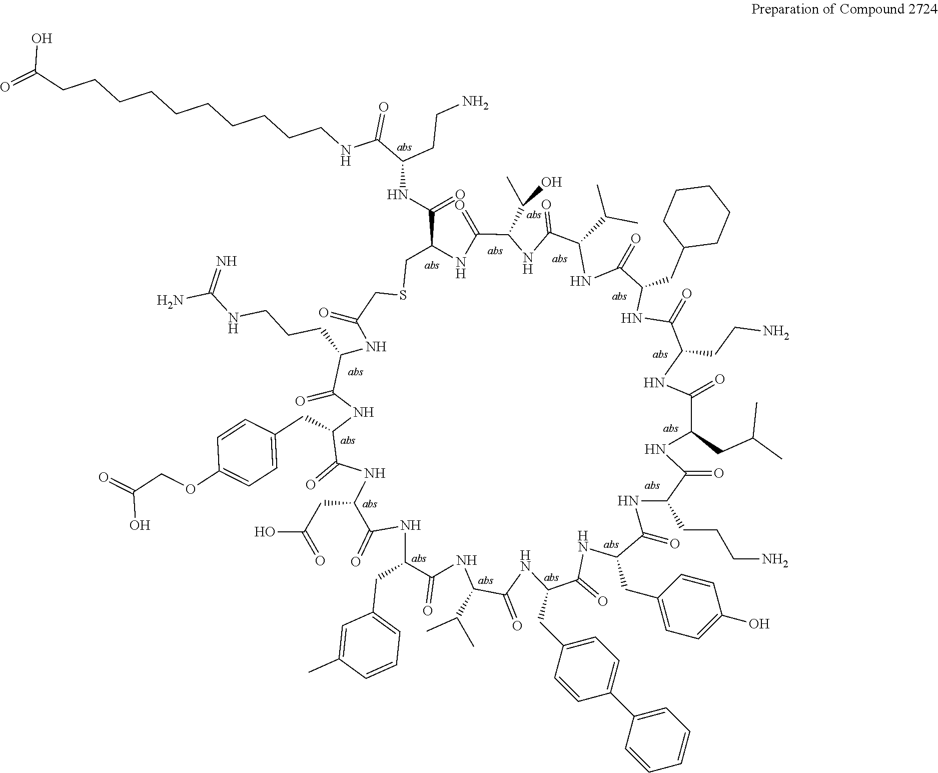 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2725 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.4 mg, and its estimated purity by LCMS analysis was 95.2%. Analysis condition A: Retention time=1.64 min; ESI-MS(+) m/z [M+2H]²⁺: 1133.2.

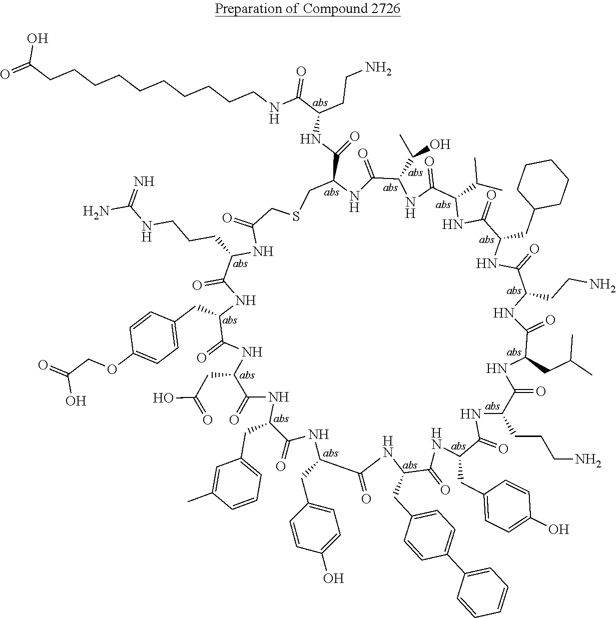 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2726 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.4 mg, and its estimated purity by LCMS analysis was 100%. Analysis condition A: Retention time=1.47 min; ESI-MS(+) m/z [M+2H]²⁺: 1165.

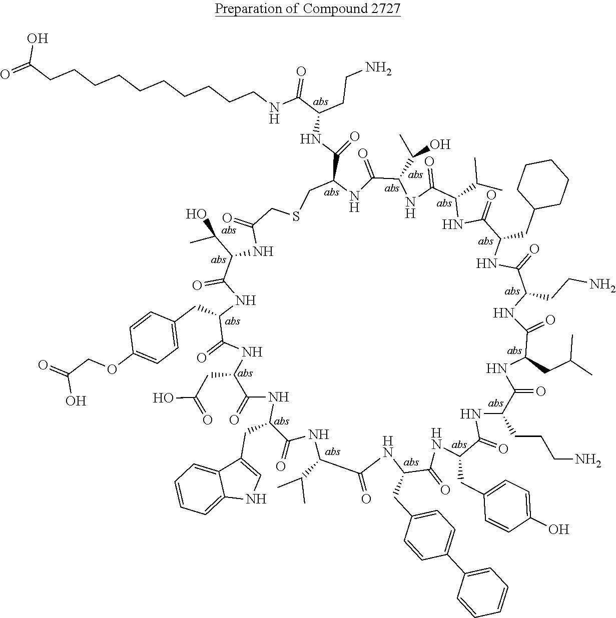 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2727 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 2-minute hold at 100% B; Flow Rate: 45 mL/min; Column Temperature: 25 C. Fraction collection was triggered centrifugal evaporation. The yield of the product was 9.3 mg, and its estimated purity by LCMS analysis was 99.2%. Analysis condition A: Retention time=1.62 min; ESI-MS(+) m/z [M+2H]²⁺: 1118.1.

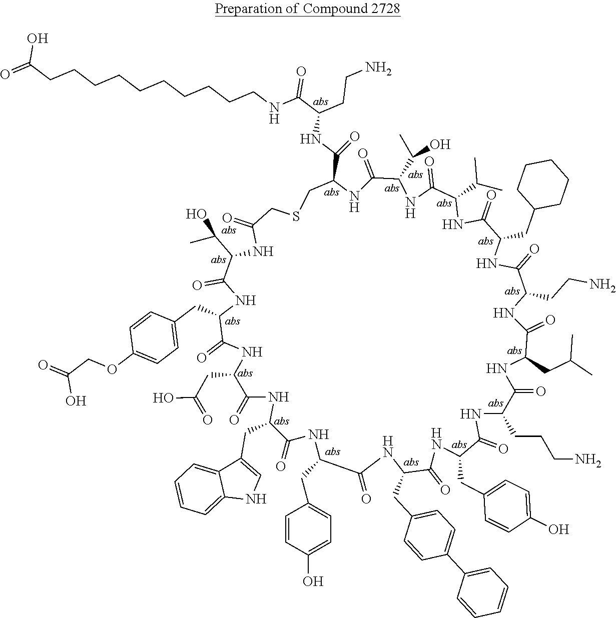 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2728 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 28.7 mg, and its estimated purity by LCMS analysis was 98.6%. Analysis condition B: Retention time=1.65 min; ESI-MS(+) m/z [M+2H]²⁺: 1150.2.

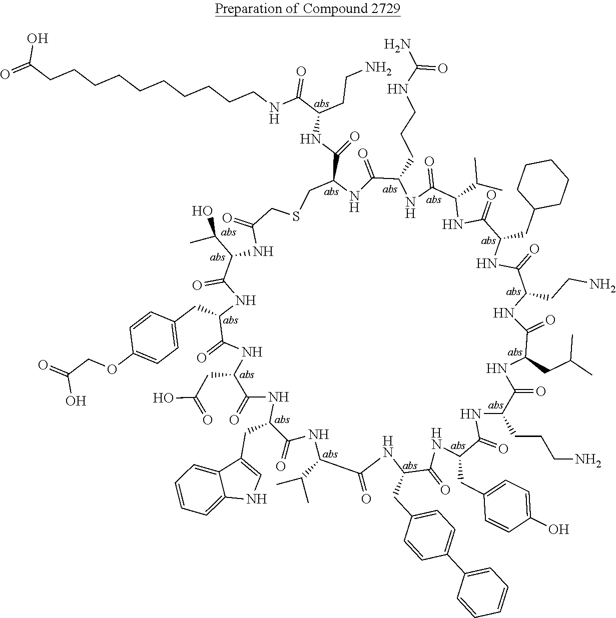 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC1=O]

Compound 2729 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.4 mg, and its estimated purity by LCMS analysis was 90.1%. Analysis condition B: Retention time=1.5 min; ESI-MS(+) m/z [M+2H]²⁺: 1146.2.

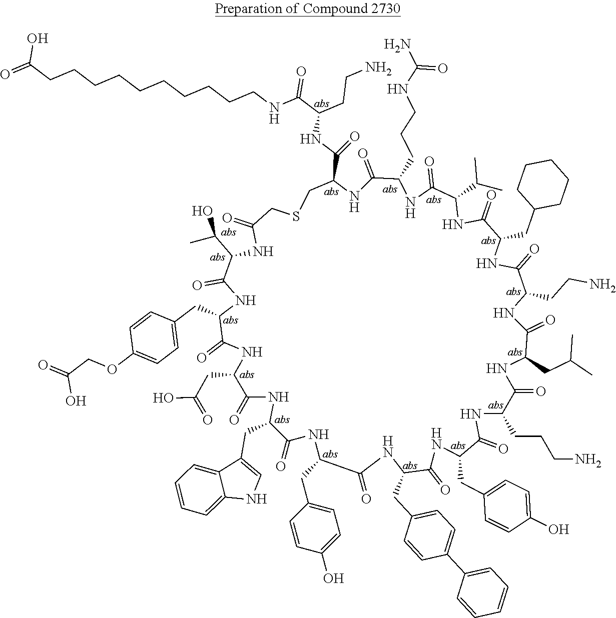 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@@H](C)O)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC1=O]

Compound 2730 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.3 mg, and its estimated purity by LCMS analysis was 96.1%. Analysis condition B: Retention time=1.45 min; ESI-MS(+) m/z [M+2H]²⁺: 1178.2.

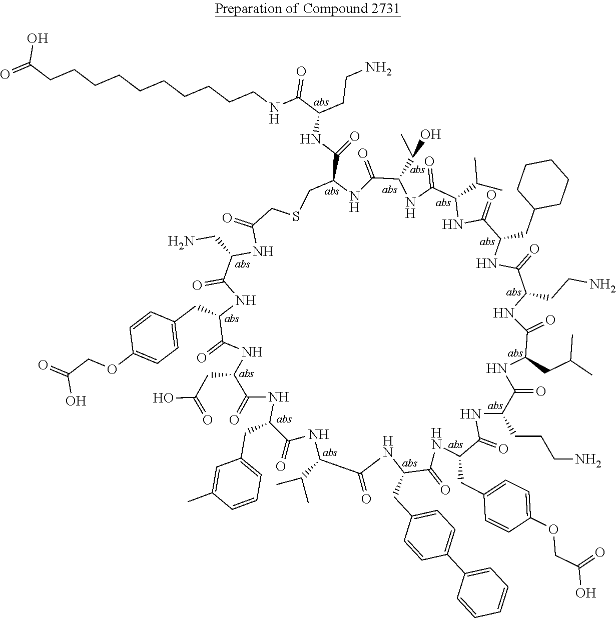 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 2731 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.7 mg, and its estimated purity by LCMS analysis was 96.8%. Analysis condition A: Retention time=1.56 min; ESI-MS(+) m/z [M+2H]²⁺: 1127.2.

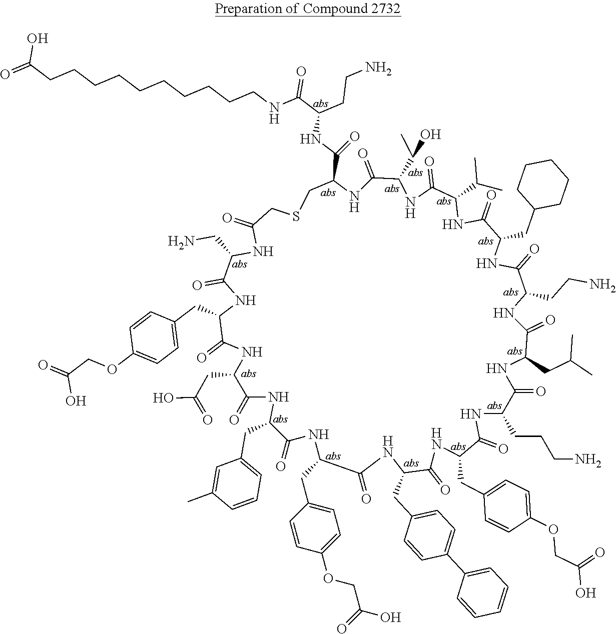 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2732 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 1.5 mg, and its estimated purity by LCMS analysis was 97.3%. Analysis condition A: Retention time=1.59 min; ESI-MS(+) m/z [M+2H]²⁺: 1188.2.

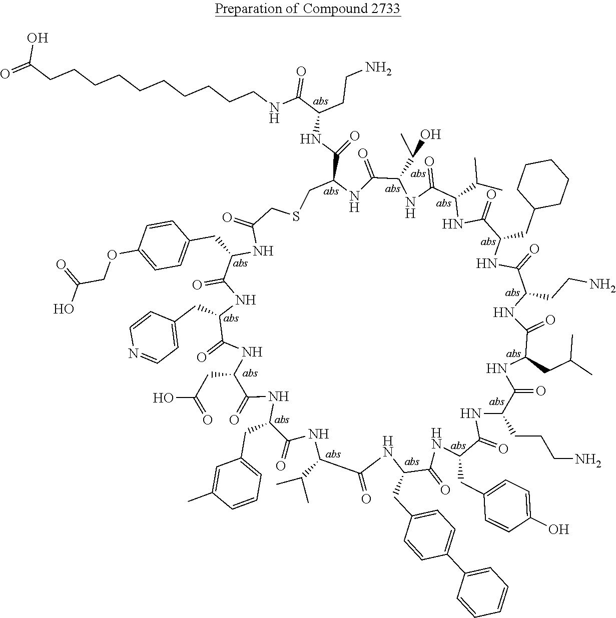 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2733 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 25% B, 25-65% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 27% B, 27-67% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.7 mg, and its estimated purity by LCMS analysis was 100%. Analysis condition A: Retention time=1.82 min; ESI-MS(+) m/z [M+2H]²⁺: 1129.2.

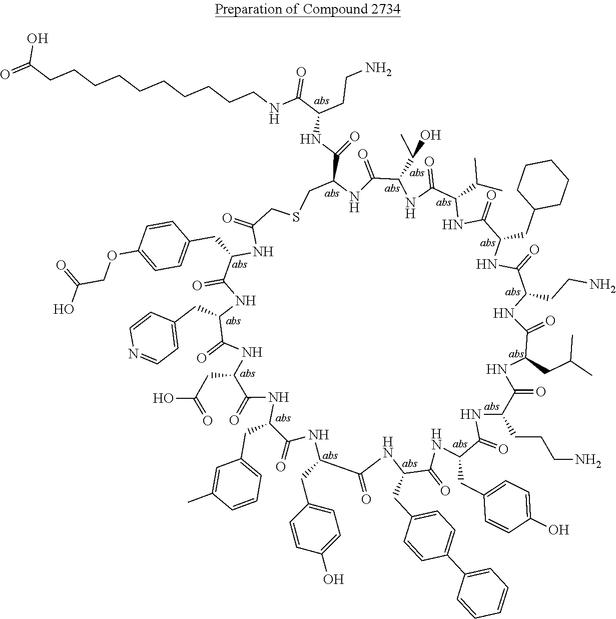 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2734 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate;

Mobile Phase B: 95:5 acetonitrile:water with 10-mM ammonium acetate; Gradient: a 0-minute hold at 24% B, 24-64% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 24% B, 24-64% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.1 mg, and its estimated purity by LCMS analysis was 100%. Analysis condition A: Retention time=1.76 min; ESI-MS(+) m/z [M+2H]²⁺: 1161.2.

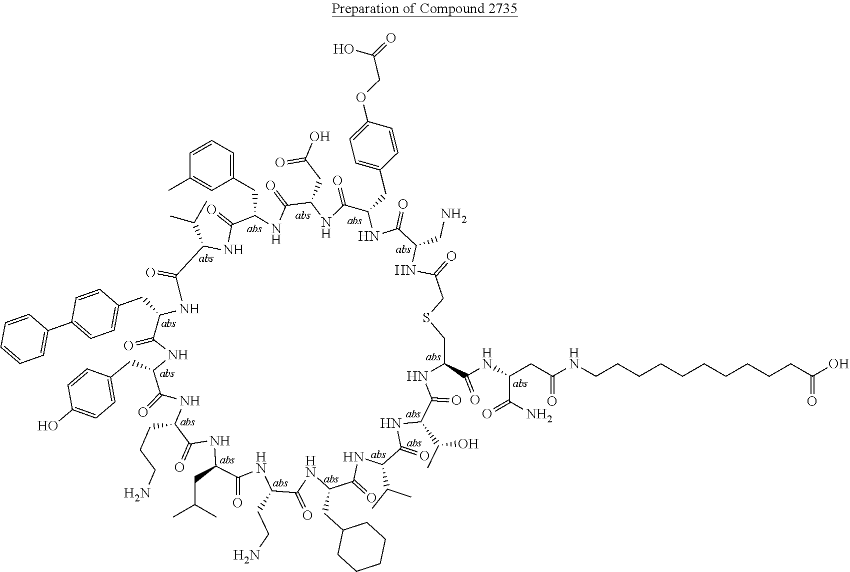 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@H](CC(=O)NCCCCCCCCCCC(=O)O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2735 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.2 mg, and its estimated purity by LCMS analysis was 95.1%. Analysis condition A: Retention time=1.66 min; ESI-MS(+) m/z [M+2H]²⁺: 1105.

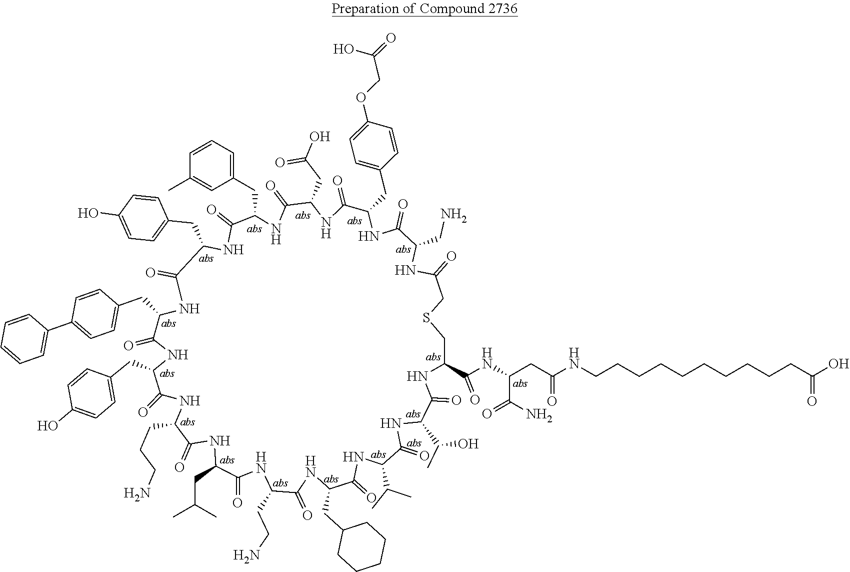 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@H](CC(=O)NCCCCCCCCCCC(=O)O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2736 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.1 mg, and its estimated purity by LCMS analysis was 95.2%. Analysis condition A: Retention time=1.66 min; ESI-MS(+) m/z [M+2H]²⁺: 1137.2.

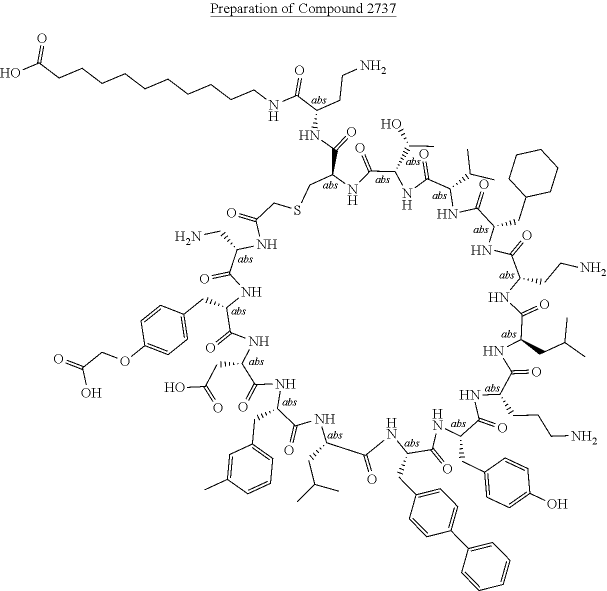 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CC(C)C)NC2=O)c1]

Compound 2737 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 24% B, 24-54% B over 28 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 6 mg, and its estimated purity by LCMS analysis was 93.8%. Analysis condition A: Retention time=1.73 min; ESI-MS(+) m/z [M+2H]²⁺: 1104.8.

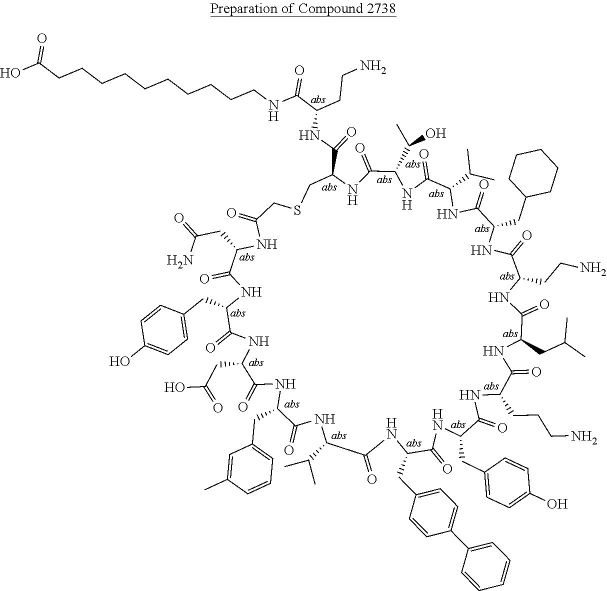 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2738 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.1 mg, and its estimated purity by LCMS analysis was 98.4%. Analysis condition B: Retention time=1.6 min; ESI-MS(+) m/z [M+2H]²⁺: 1083.2.

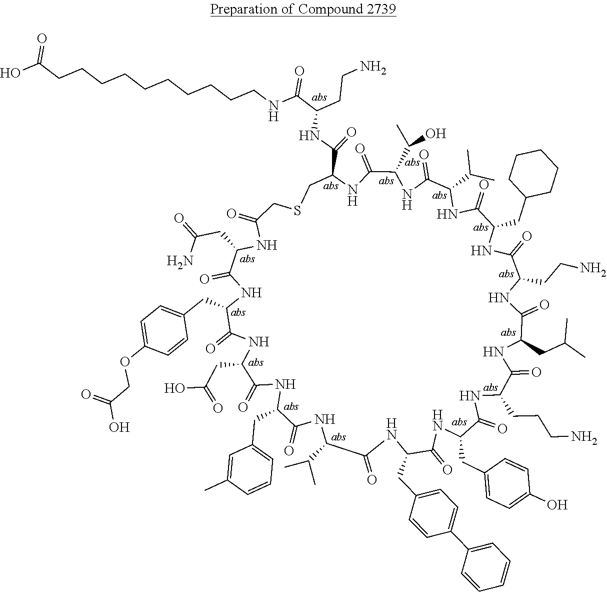 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2739 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-62% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 6 mg, and its estimated purity by LCMS analysis was 100%. Analysis condition A: Retention time=1.74 min; ESI-MS(+) m/z [M+2H]²⁺: 1111.9.

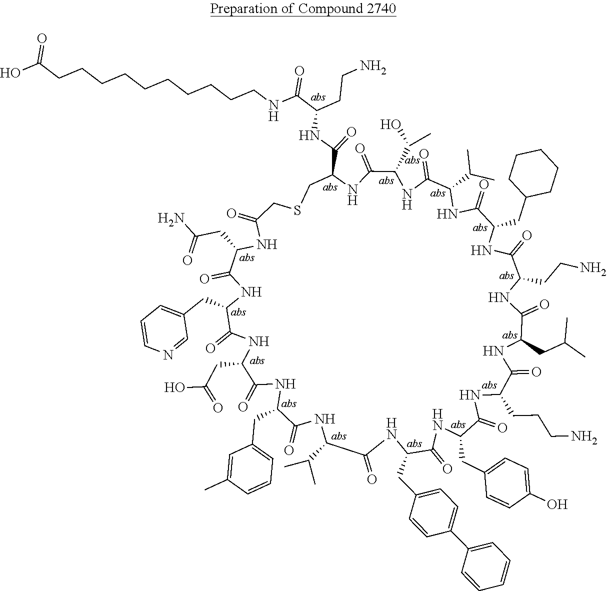 [SMILES: Cc1cccc(C[C@@H]2NC(=O)[C@H](CC(=O)O)NC(=O)[C@H](Cc3cccnc3)NC(=O)[C@H](CC(N)=O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2740 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4.5 mg, and its estimated purity by LCMS analysis was 100%. Analysis condition A: Retention time=1.81 min; ESI-MS(+) m/z [M+3H]³⁺: 717.4.

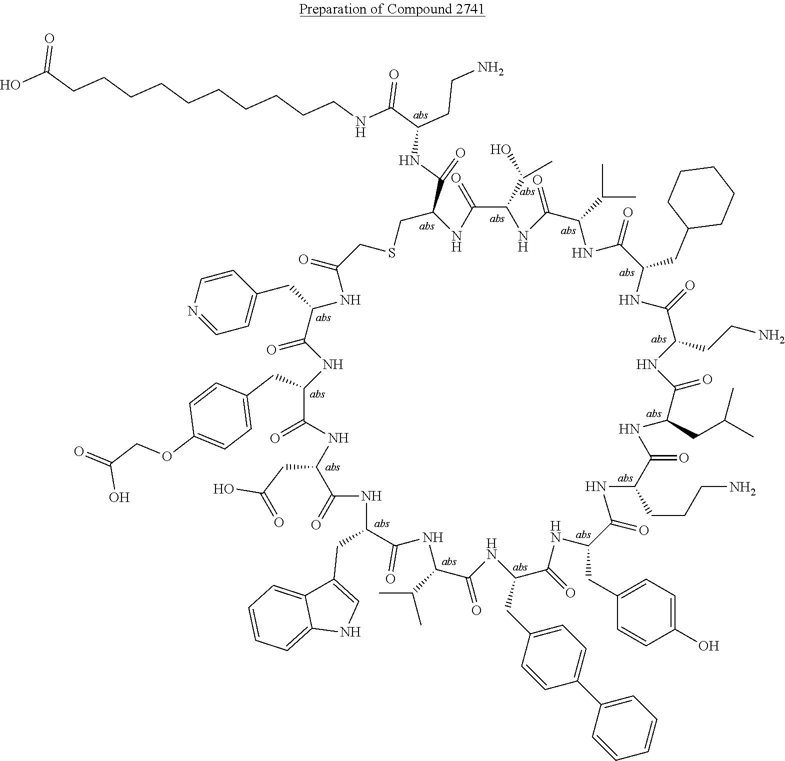 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2741 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 5.3 mg, and its estimated purity by LCMS analysis was 95.3%. Analysis condition A: Retention time=1.6 min; ESI-MS(+) m/z [M+2H]²⁺: 1142.

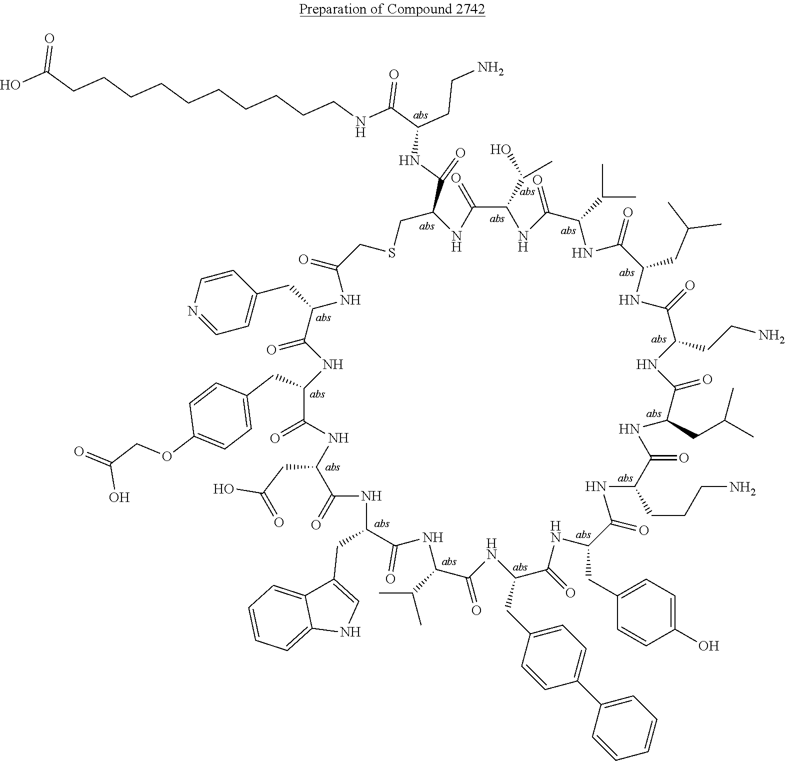 [SMILES: CC(C)C[C@@H]1NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC1=O]

Compound 2742 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2.7 mg, and its estimated purity by LCMS analysis was 100%. Analysis condition A: Retention time=1.46 min; ESI-MS(+) m/z [M+3H]³⁺: 747.8.

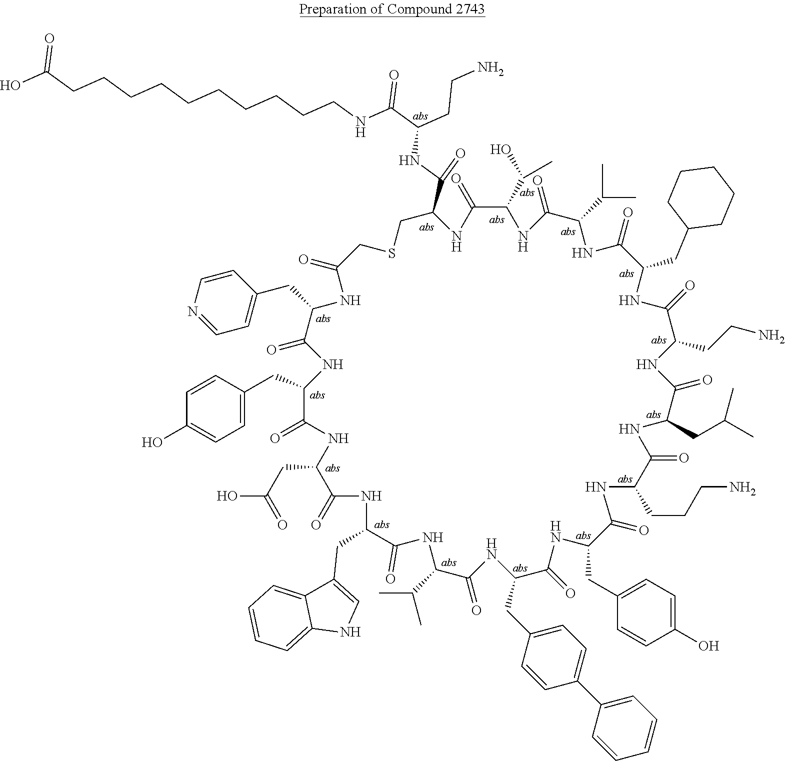 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2743 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-51% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.3 mg, and its estimated purity by LCMS analysis was 90.8%. Analysis condition B: Retention time=1.55 min; ESI-MS(+) m/z [M+3H]³⁺: 742.

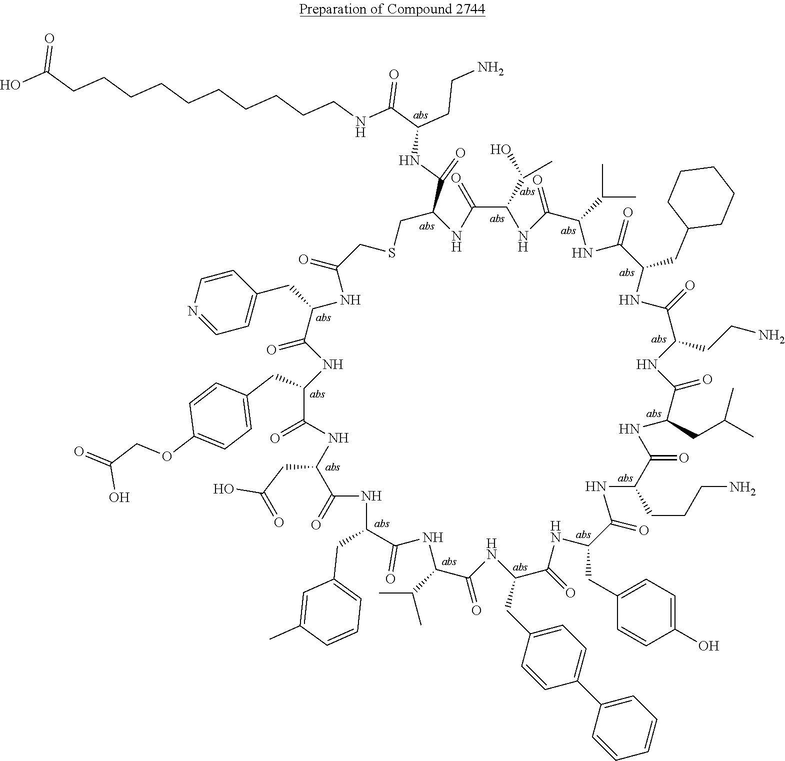 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2744 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-52% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 3.4 mg, and its estimated purity by LCMS analysis was 96.5%. Analysis condition A: Retention time=1.61 min; ESI-MS(+) m/z [M+2H]²⁺: 1129.2.

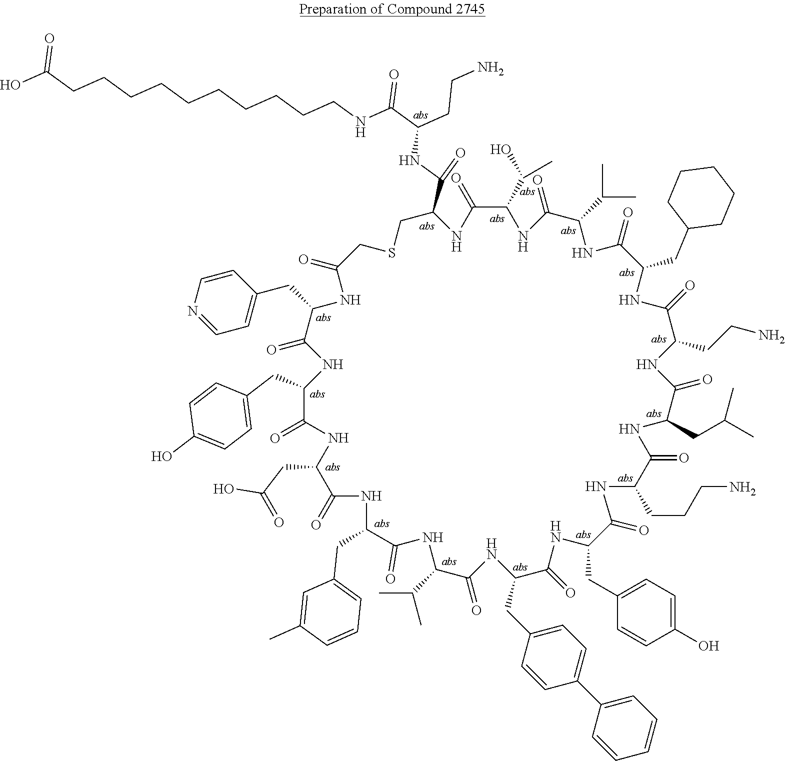 [SMILES: Cc1cccc(C[C@@H]2NC(=O)[C@H](CC(=O)O)NC(=O)[C@H](Cc3ccc(O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2745 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 7.5 mg, and its estimated purity by LCMS analysis was 93.1%. Analysis condition A: Retention time=1.69 min; ESI-MS(+) m/z [M+2H]²⁺: 1100.

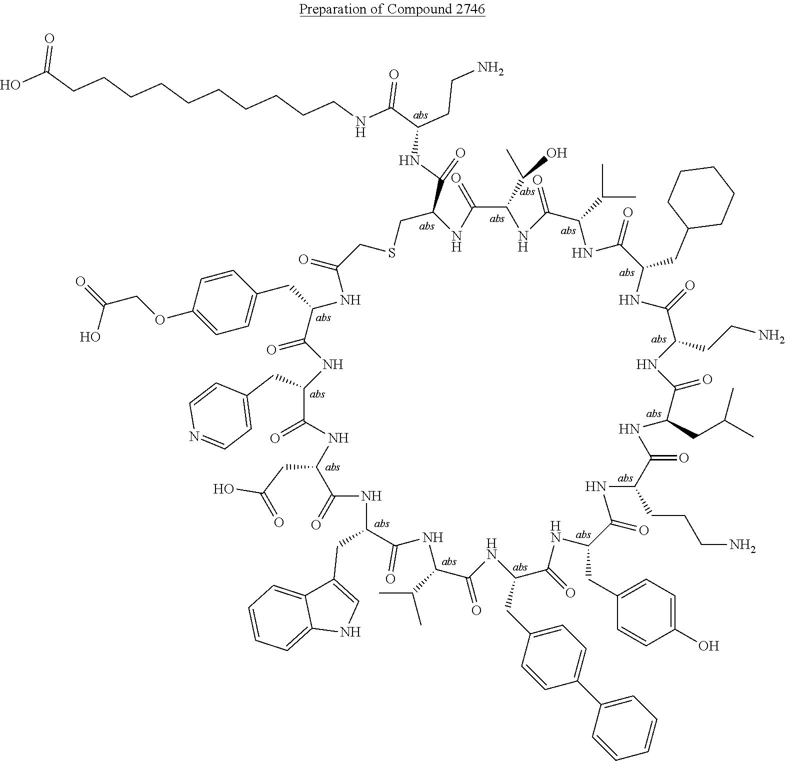 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2746 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-50% B over 35 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4.4 mg, and its estimated purity by LCMS analysis was 96.6%. Analysis condition A: Retention time=1.7 min; ESI-MS(+) m/z [M+3H]³⁺: 761.2.

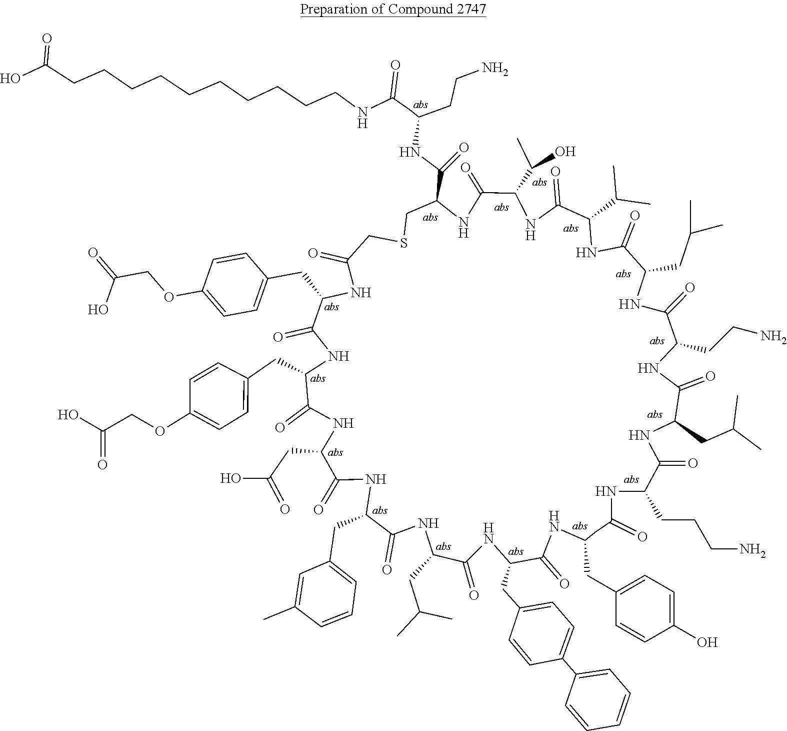 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)CC(=O)NCCCCCCCCCCC(=O)O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](CC(C)C)NC2=O)c1]

Compound 2747 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-50% B over 28 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The yield of the product was 31.3 mg, and its estimated purity by LCMS analysis was 97.7%. Analysis condition B: Retention time=1.56 min; ESI-MS(+) m/z [M+2H]²⁺: 1152.2.

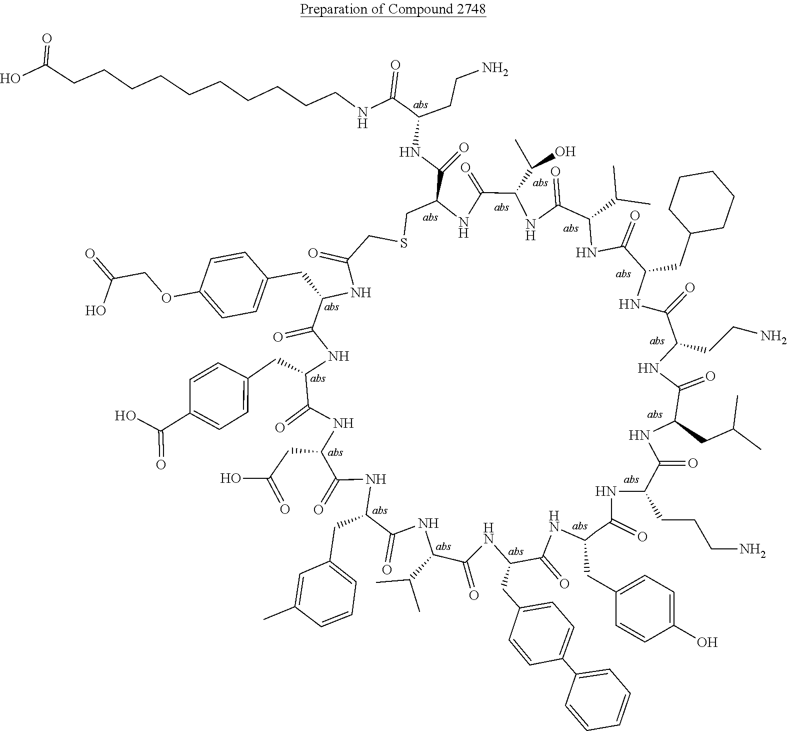 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)CC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](C(C)C)NC2=O)c1]

Compound 2748 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-50% B over 35 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 2.2 mg, and its estimated purity by LCMS analysis was 97.2%. Analysis condition B: Retention time=1.74 min; ESI-MS(+) m/z [M+2H]²⁺: 1150.2.

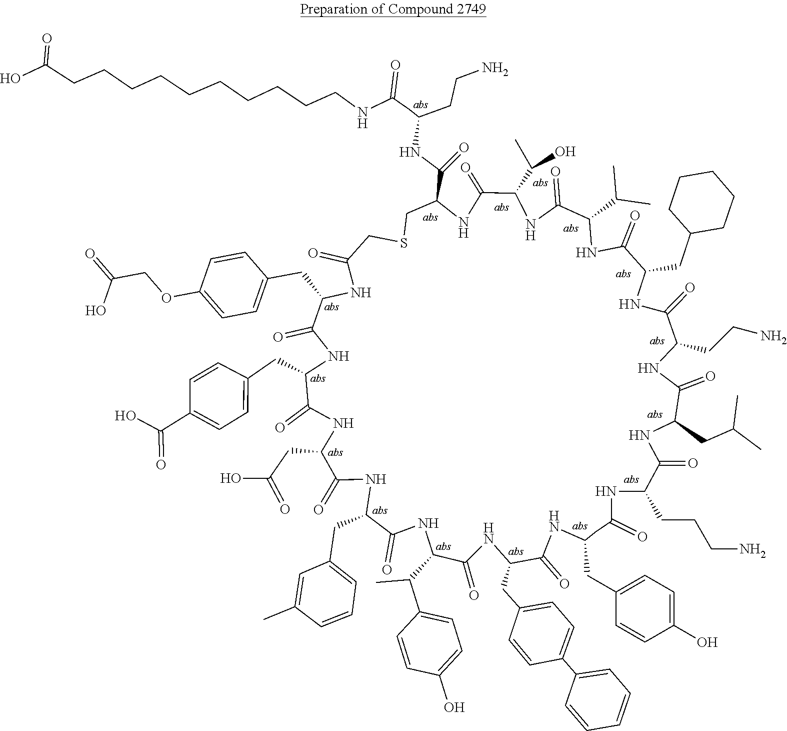 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)CC(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)C[C@H](Cc3ccc(O)cc3)NC(=O)[C@H](Cc3ccc(-c4ccccc4)cc3)NC(=O)[C@H](C(C)c3ccc(O)cc3)NC2=O)c1]

Compound 2749 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 3.4 mg, and its estimated purity by LCMS analysis was 90.6%. Analysis condition A: Retention time=1.63 min; ESI-MS(+) m/z [M+3H]³⁺: 788.5.

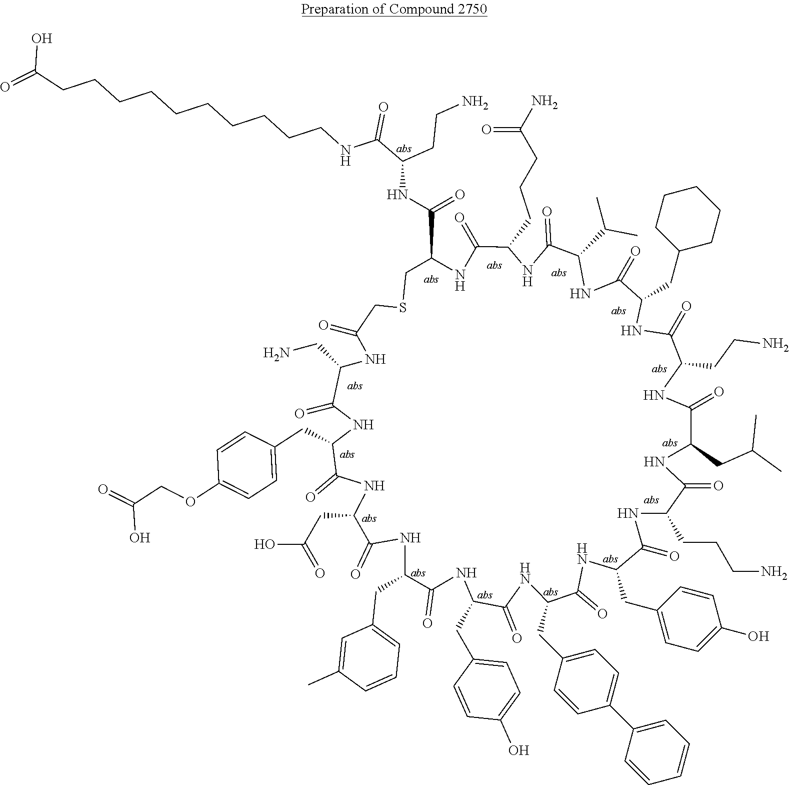 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2750 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 25 minutes, then a 5-minute hold at 100% B; Flow Rate: 20 mL/min; Column Temperature: 25 C. Fraction collection was triggered by MS signals. Fractions containing the desired product were combined and dried via centrifugal evaporation. The yield of the product was 27.2 mg, and its estimated purity by LCMS analysis was 96.1%. Analysis condition A: Retention time=1.47 min; ESI-MS(+) m/z [M+2H]²⁺: 1150.5.

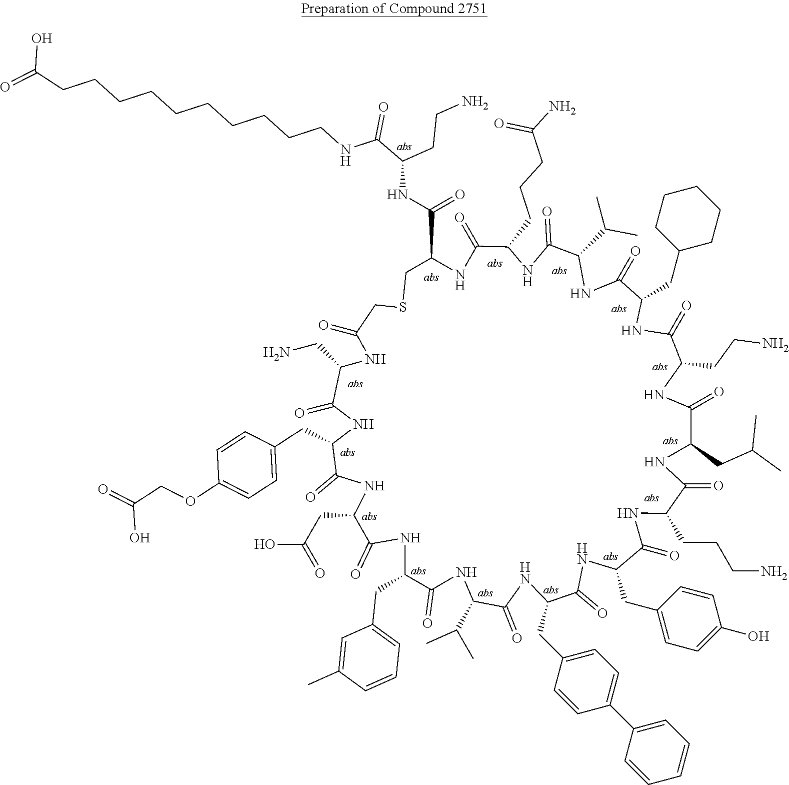 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2751 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 14.8 mg, and its estimated purity by LCMS analysis was 97.5%. Analysis condition A: Retention time=1.58 min; ESI-MS(+) m/z [M+3H]³⁺: 746.1.

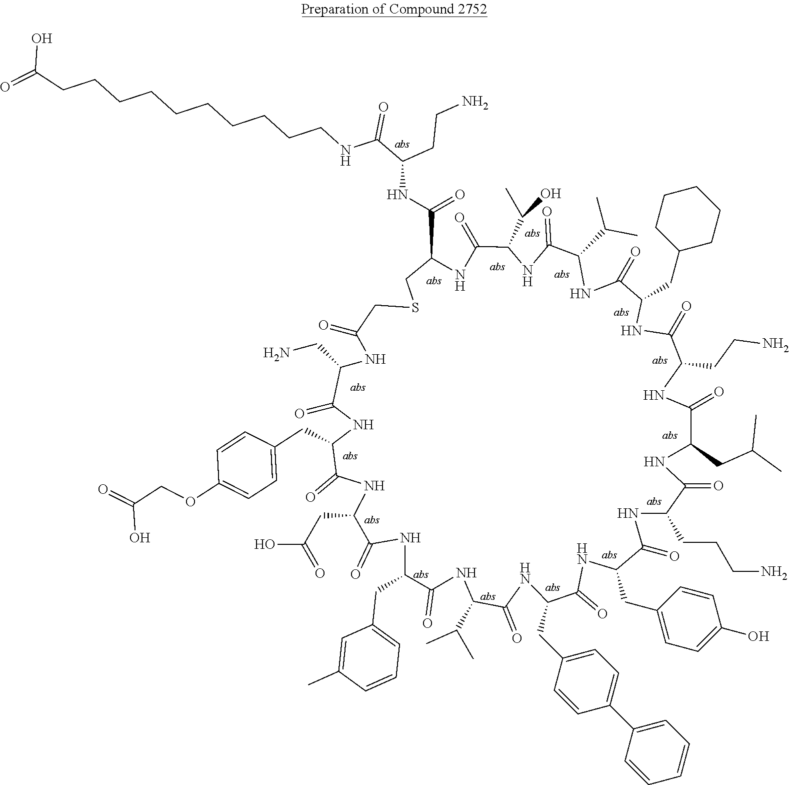 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2752 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.4 mg, and its estimated purity by LCMS analysis was 90.2%. Analysis condition A: Retention time=1.65 min; ESI-MS(+) m/z [M+2H]²⁺: 1105.

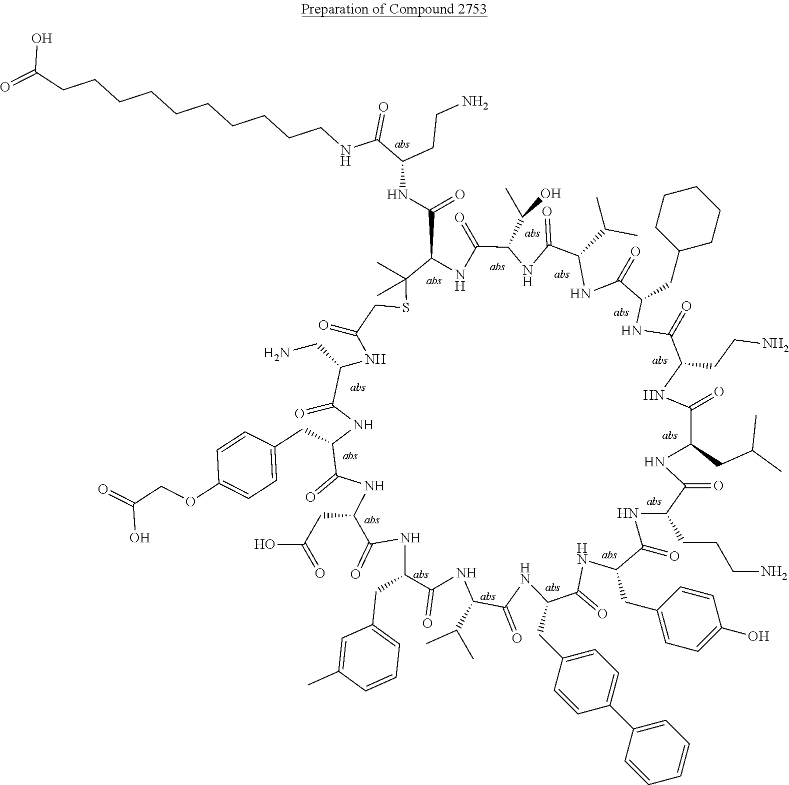 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC(C)(C)[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2753 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13 mg, and its estimated purity by LCMS analysis was 94.7%. Analysis condition B: Retention time=1.64 min; ESI-MS(+) m/z [M+2H]²⁺: 1112.3.

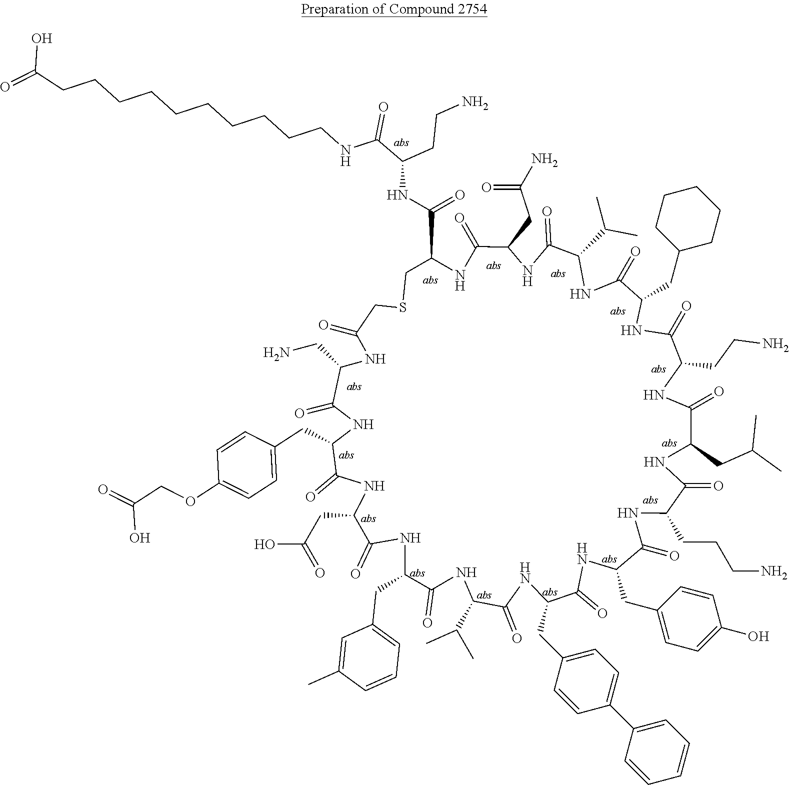 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2754 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 30.2 mg, and its estimated purity by LCMS analysis was 90%. Analysis condition B: Retention time=1.6 min; ESI-MS(+) m/z [M+3H]³⁺: 737.

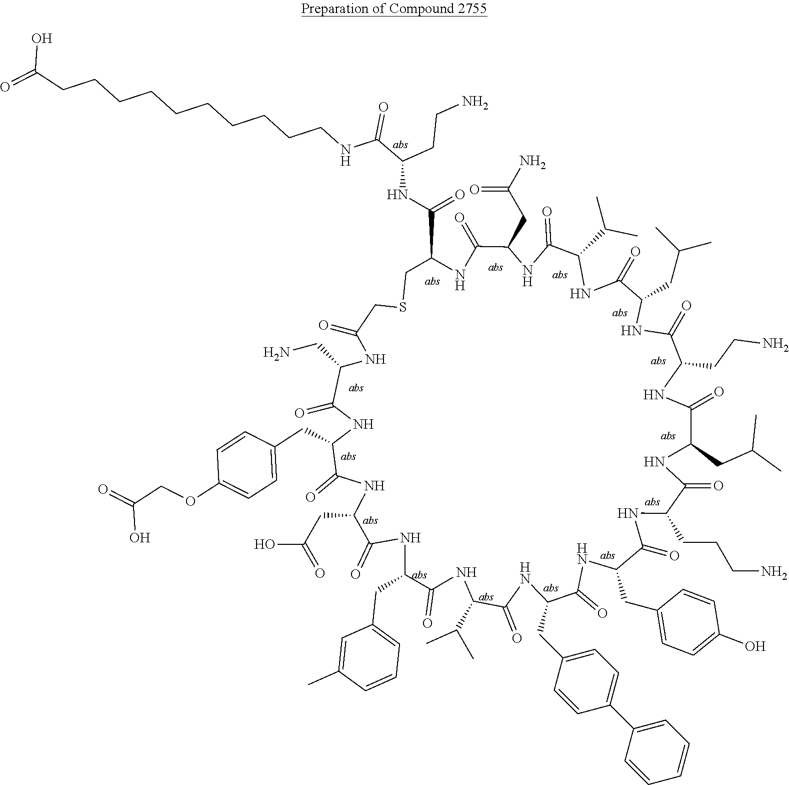 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CC(N)=O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2755 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 21.5 mg, and its estimated purity by LCMS analysis was 93.3%. Analysis condition B: Retention time=1.46 min; ESI-MS(+) m/z [M+3H]³⁺: 723.

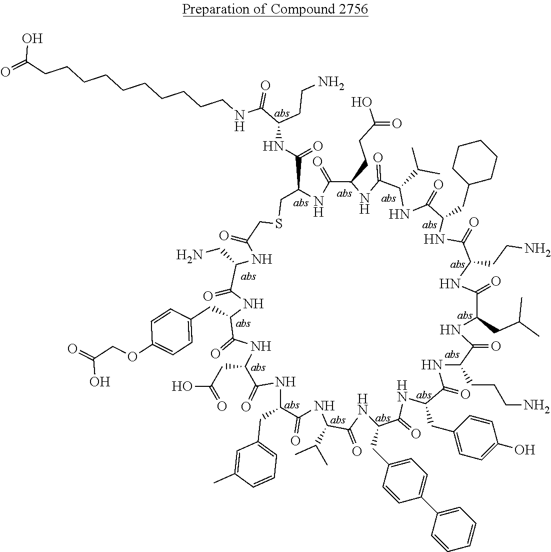 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2756 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.6 mg, and its estimated purity by LCMS analysis was 90.7%. Analysis condition A: Retention time=1.67 min; ESI-MS(+) m/z [M+2H]²⁺: 1112.2.

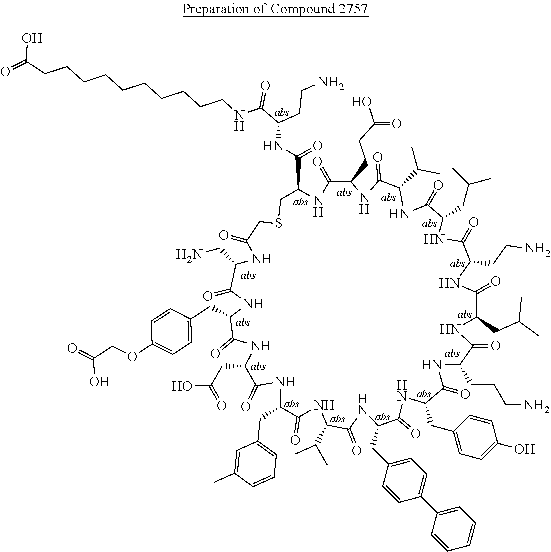 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CCC(=O)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2757 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.9 mg, and its estimated purity by LCMS analysis was 97.6%. Analysis condition B: Retention time=1.46 min; ESI-MS(+) m/z [M+2H]²⁺: 1091.92.

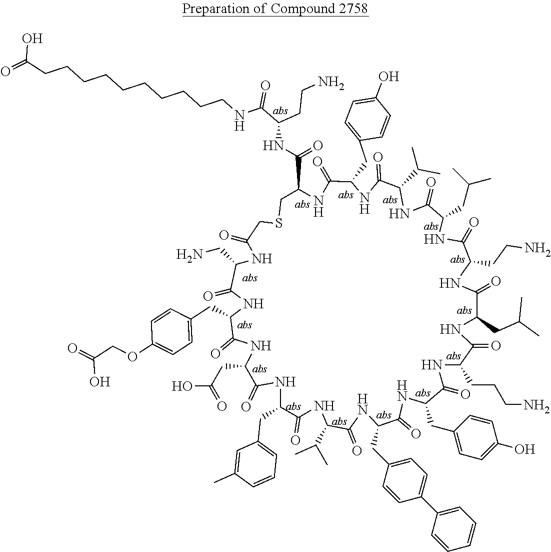 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3ccc(O)cc3)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2758 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at centrifugal evaporation. The yield of the product was 4.1 mg, and its estimated purity by LCMS analysis was 95.4%. Analysis condition A: Retention time=1.43 min; ESI-MS(+) m/z [M+2H]²⁺: 1109.1.

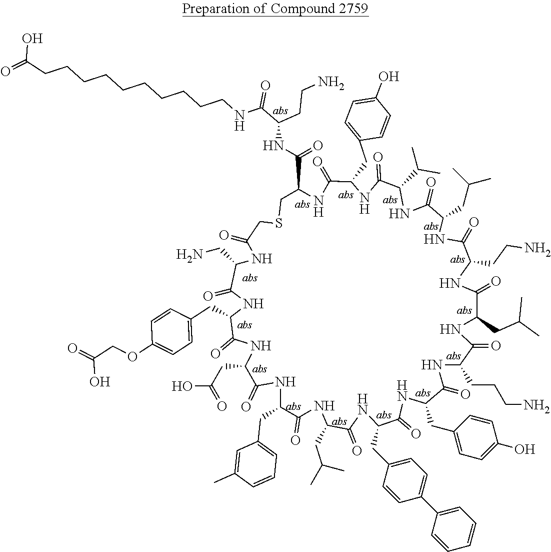 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3ccc(O)cc3)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CC(C)C)NC2=O)c1]

Compound 2759 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.1 mg, and its estimated purity by LCMS analysis was 97.3%. Analysis condition B: Retention time=1.52 min; ESI-MS(+) m/z [M+2H]²⁺: 1116.

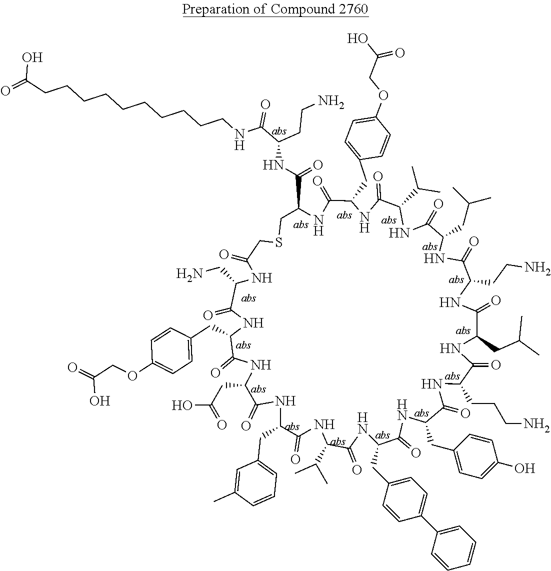 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3ccc(OCC(=O)O)cc3)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2760 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.5 mg, and its estimated purity by LCMS analysis was 90.7%. Analysis condition A: Retention time=1.48 min; ESI-MS(+) m/z [M+2H]²⁺: 1138.3.

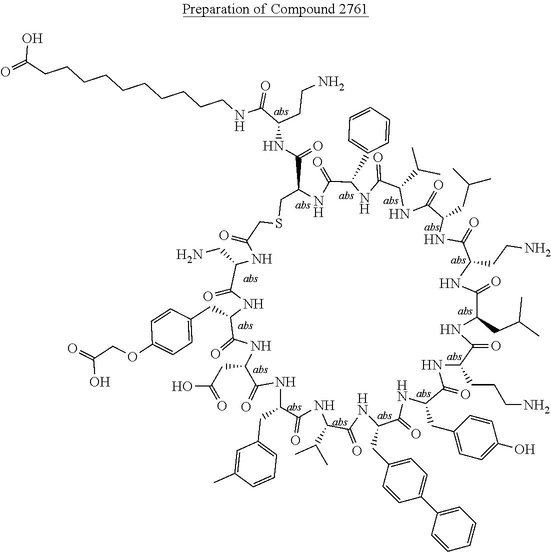 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](c3ccccc3)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2761 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 10% B, 10-50% B over 20 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 35 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.2 mg, and its estimated purity by LCMS analysis was 100%. Analysis condition A: Retention time=1.47 min; ESI-MS(+) m/z [M+2H]²⁺: 1094.

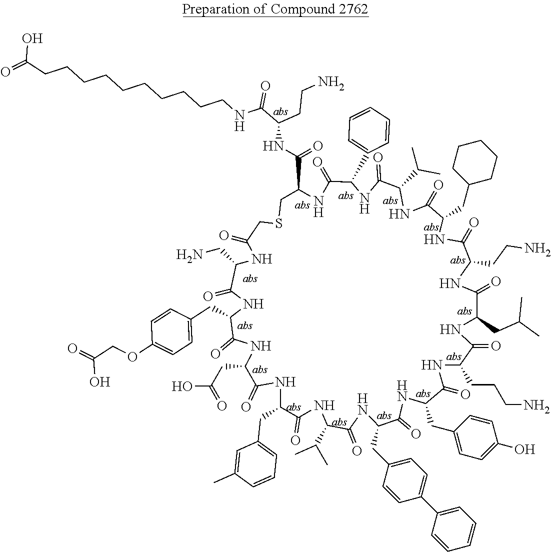 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](c3ccccc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2762 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.5 mg, and its estimated purity by LCMS analysis was 100%. Analysis condition A: Retention time=1.74 min; ESI-MS(+) m/z [M+2H]²⁺: 1114.2.

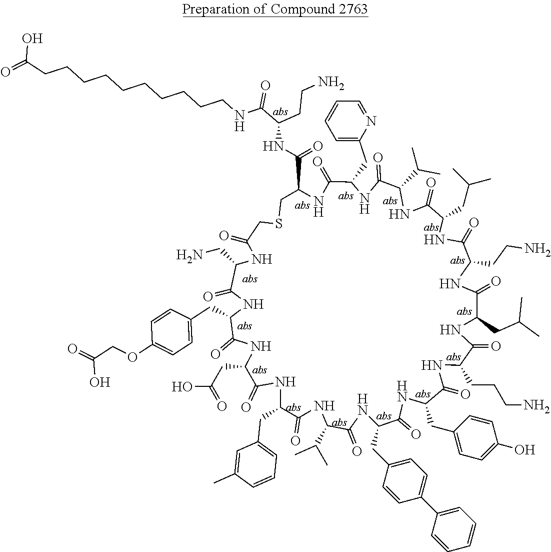 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3ccccn3)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2763 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at centrifugal evaporation. The yield of the product was 11.1 mg, and its estimated purity by LCMS analysis was 97.8%. Analysis condition A: Retention time=1.48, 1.51 min; ESI-MS(+) m/z [M+3H]³⁺: 735.7, 735.7.

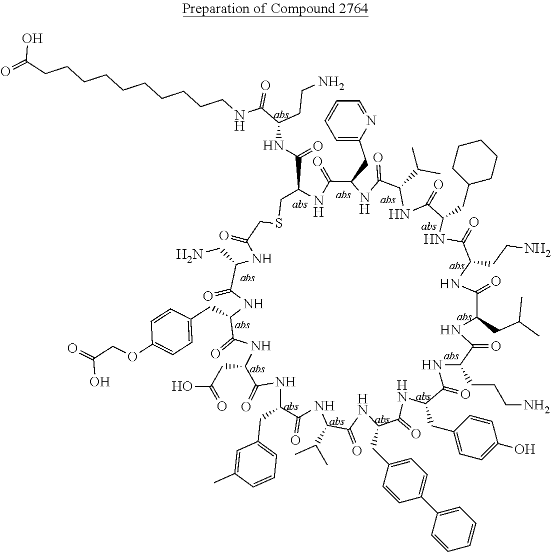 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](Cc3ccccn3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2764 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.8 mg, and its estimated purity by LCMS analysis was 95.3%. Analysis condition A: Retention time=1.59 min; ESI-MS(+) m/z [M+3H]³⁺: 748.1.

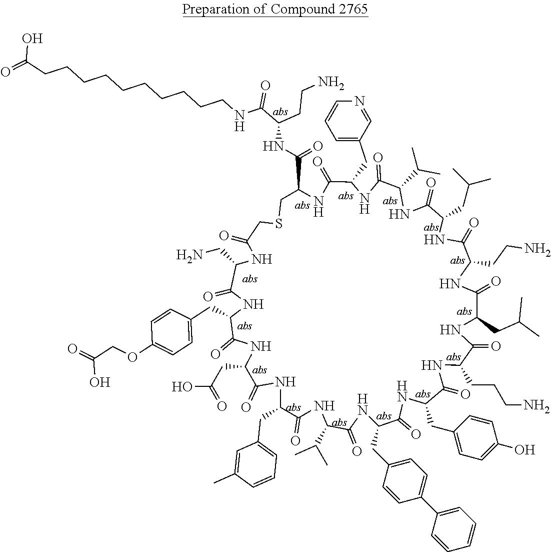 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3cccnc3)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2765 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 11% B, 11-51% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.4 mg, and its estimated purity by LCMS analysis was 100%. Analysis condition A: Retention time=1.54 min; ESI-MS(+) m/z [M+3H]³⁺: 734.4.

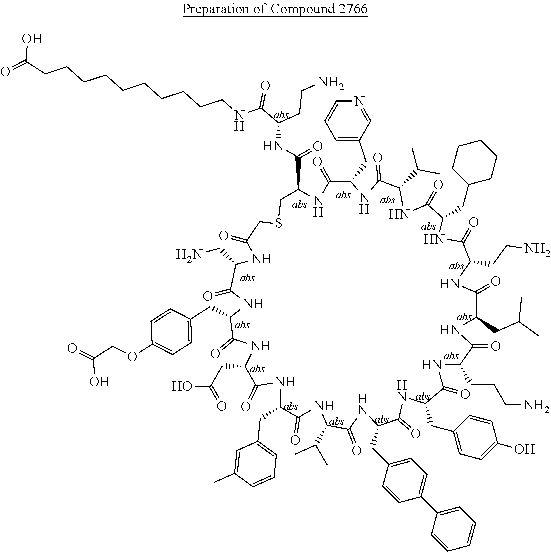 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3cccnc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2766 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.8 mg, and its estimated purity by LCMS analysis was 93.8%. Analysis condition B: Retention time=1.51 min; ESI-MS(+) m/z [M+3H]³⁺: 748.1.

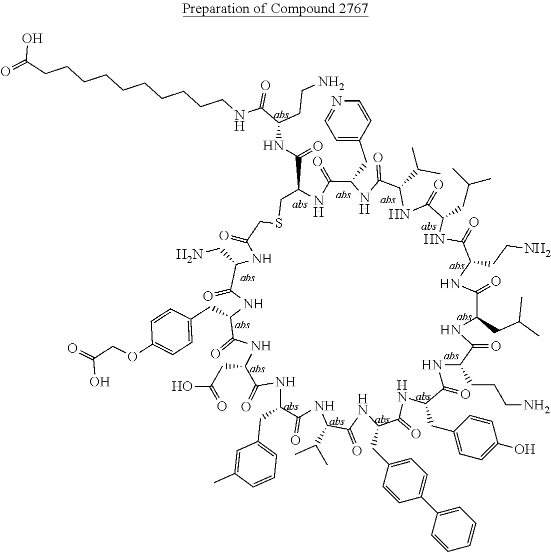 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3ccncc3)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2767 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 10% B, 10-50% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 35 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.7 mg, and its estimated purity by LCMS analysis was 97.8%. Analysis condition B: Retention time=1.41 min; ESI-MS(+) m/z [M+2H]²⁺: 1101.2.

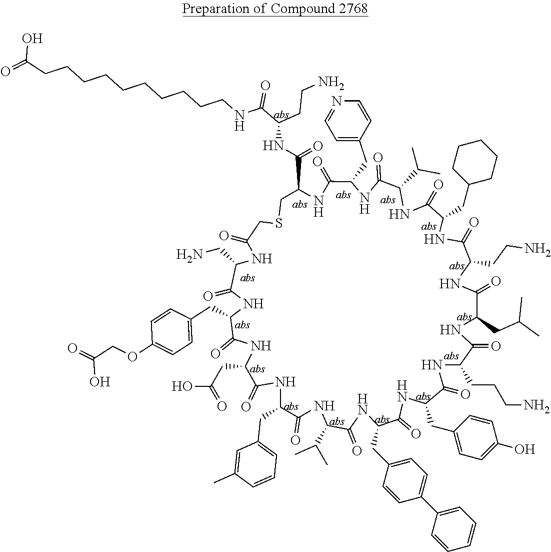 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3ccncc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2768 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at by MS and UV signals. Fractions containing the desired product were combined and dried via centrifugal evaporation. The yield of the product was 1.8 mg, and its estimated purity by LCMS analysis was 100%. Analysis condition A: Retention time=1.58 min; ESI-MS(+) m/z [M+2H]²⁺: 1121.

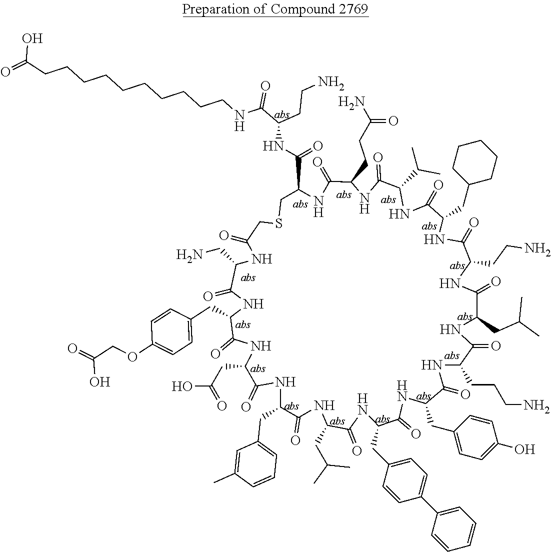 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CC(C)C)NC2=O)c1]

Compound 2769 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 45.6 mg, and its estimated purity by LCMS analysis was 97.3%. Analysis condition A: Retention time=1.79 min; ESI-MS(+) m/z [M+3H]³⁺: 746.1.

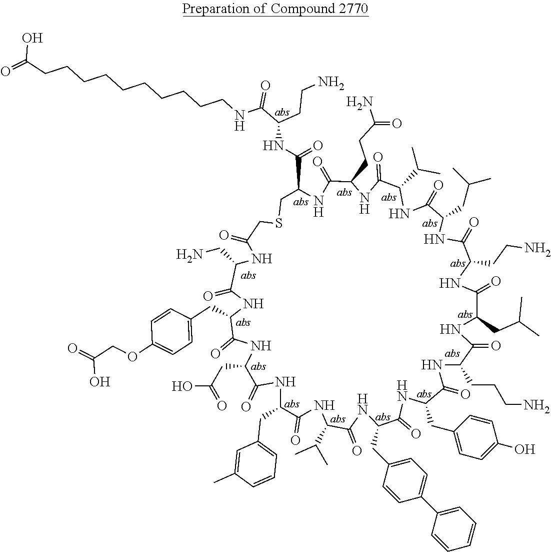 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H](CCC(N)=O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2770 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 10% B, 10-40% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 32.7 mg, and its estimated purity by LCMS analysis was 94.7%. Analysis condition B: Retention time=1.43 min; ESI-MS(+) m/z [M+3H]³⁺: 728.

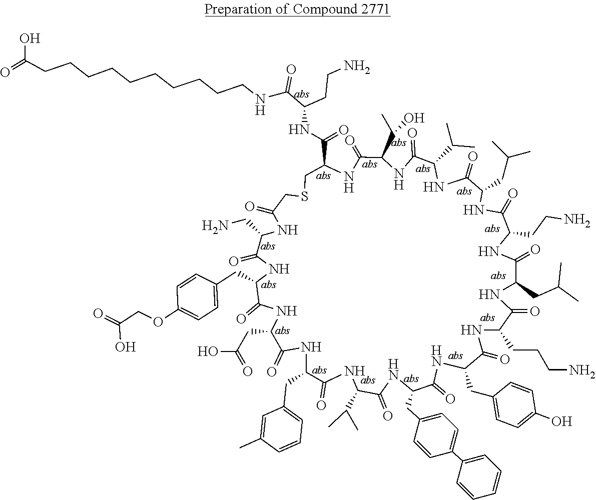 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@@H]([C@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2771 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 11% B, 11-51% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.2 mg, and its estimated purity by LCMS analysis was 100%. Analysis condition A: Retention time=1.51 min; ESI-MS(+) m/z [M+2H]²⁺: 1078.3.

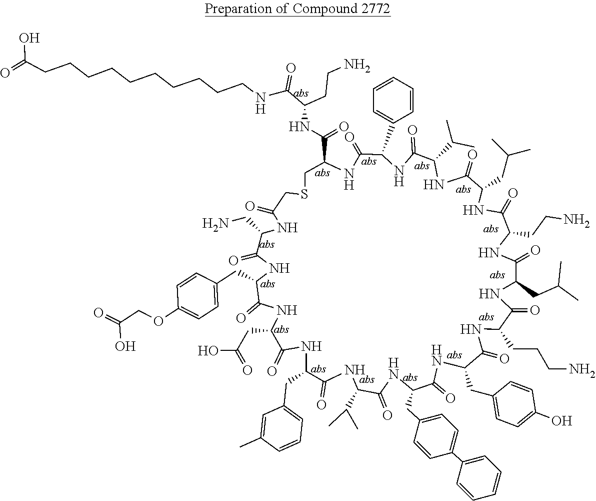 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](c3ccccc3)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2772 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 10% B, 10-50% B over 20 minutes, then a 6-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.1 mg, and its estimated purity by LCMS analysis was 93.9%. Analysis condition B: Retention time=1.54 min; ESI-MS(+) m/z [M+2H]²⁺: 1094.3.

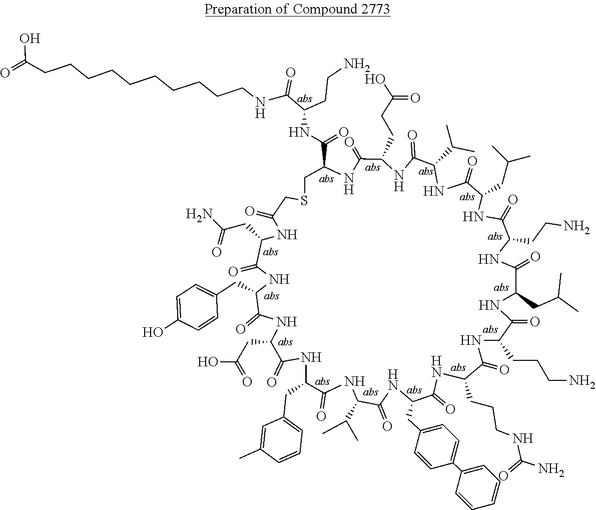 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2773 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 8.4 mg, and its estimated purity by LCMS analysis was 94.8%. Analysis condition B: Retention time=1.53 min; ESI-MS(+) m/z [M+2H]²⁺: 1074.

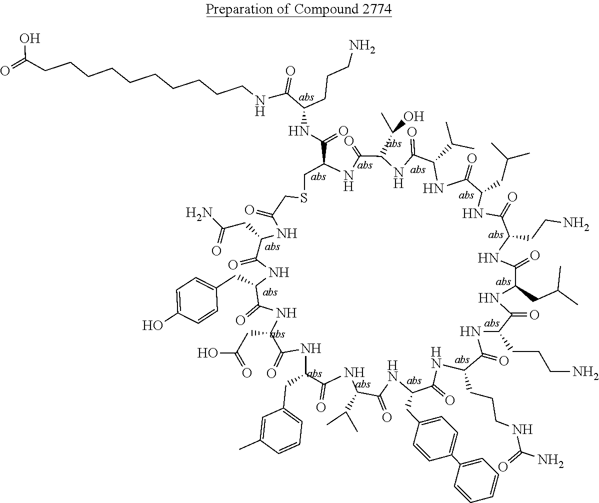

Compound 2774 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.9 mg, and its estimated purity by LCMS analysis was 93.1%. Analysis condition B: Retention time=1.42 min; ESI-MS(+) m/z [M+3H]³⁺: 712.1.

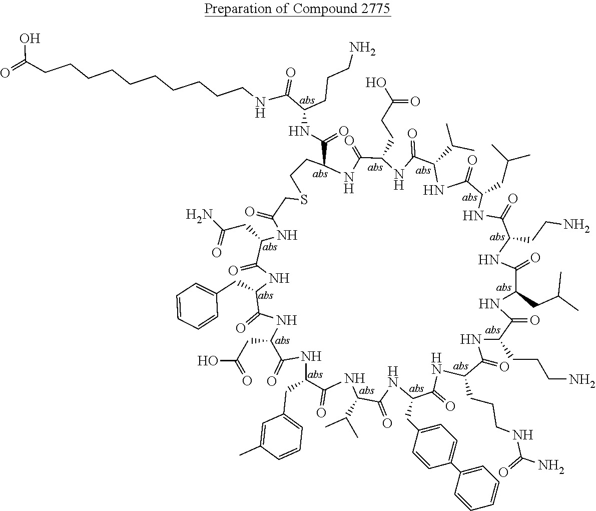 [SMILES: Cc1cccc(C[C@@H]2NC(=O)[C@H](CC(=O)O)NC(=O)[C@H](Cc3ccccc3)NC(=O)[C@H](CC(N)=O)NC(=O)CSCC[C@@H](C(=O)N[C@@H](CCCN)C(=O)NCCCCCCCCCCC(=O)O)NC(=O)[C@H](CCC(=O)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2775 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at centrifugal evaporation. The yield of the product was 20.3 mg, and its estimated purity by LCMS analysis was 96.4%. Analysis condition B: Retention time=1.52 min; ESI-MS(+) m/z [M+2H]²⁺: 1080.2.

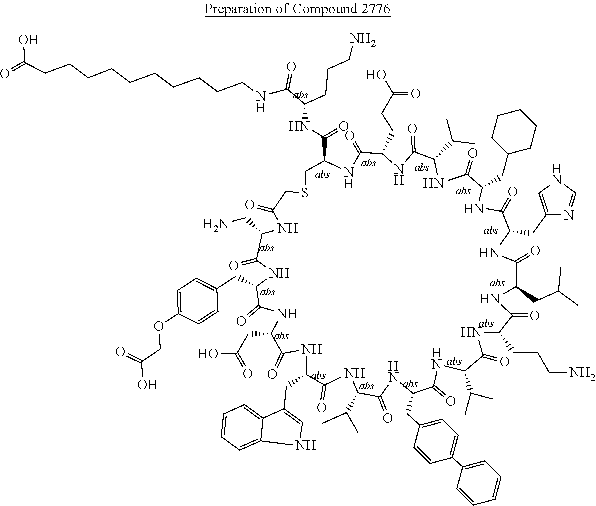 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](Cc2c[nH]cn2)NC1=O]

Compound 2776 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.4 mg, and its estimated purity by LCMS analysis was 99.4%. Analysis condition B: Retention time=1.52 min; ESI-MS(+) m/z [M+2H]²⁺: 1118.2.

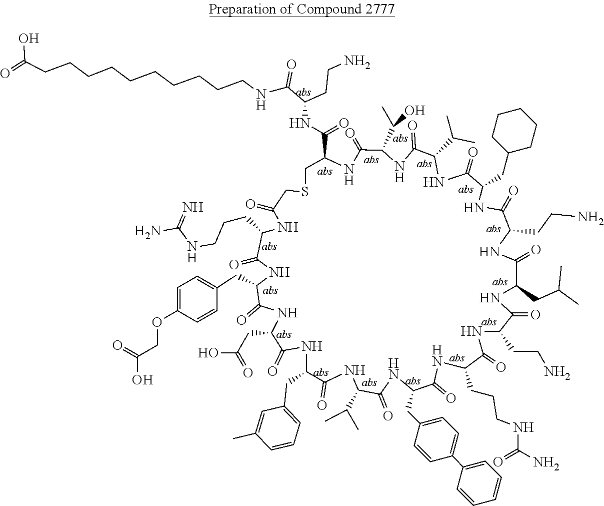

Compound 2777 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 13.6 mg, and its estimated purity by LCMS analysis was 94.9%. Analysis condition B: Retention time=1.58 min; ESI-MS(+) m/z [M+2H]²⁺: 1123.2.

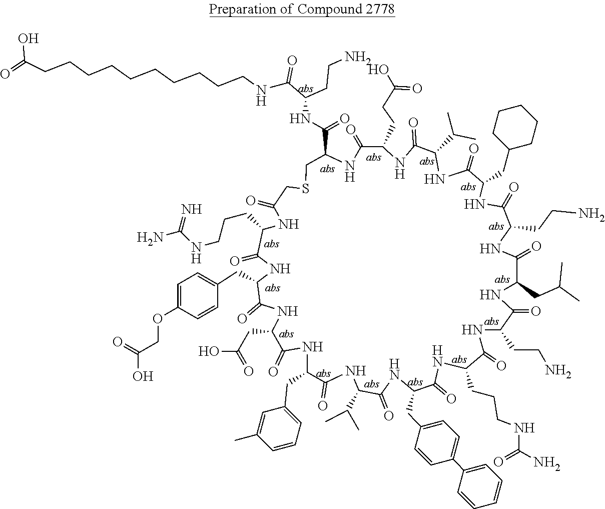

Compound 2778 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.5 mg, and its estimated purity by LCMS analysis was 96.6%. Analysis condition A: Retention time=1.6 min; ESI-MS(+) m/z [M+2H]²⁺: 1137.3.

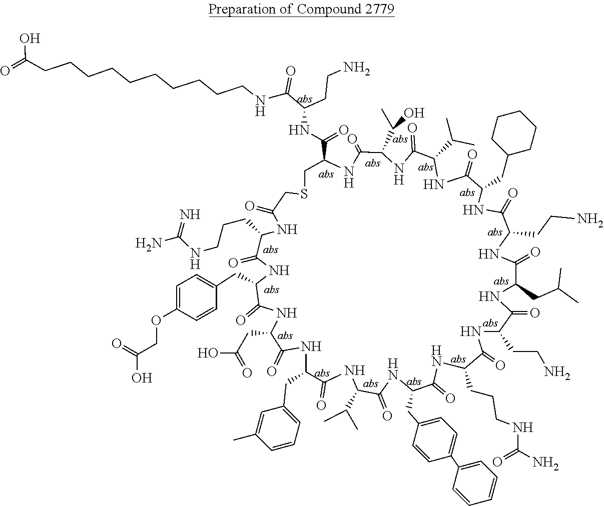

Compound 2779 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.9 mg, and its estimated purity by LCMS analysis was 98.1%. Analysis condition A: Retention time=1.6 min; ESI-MS(+) m/z [M+2H]²⁺: 1130.1.

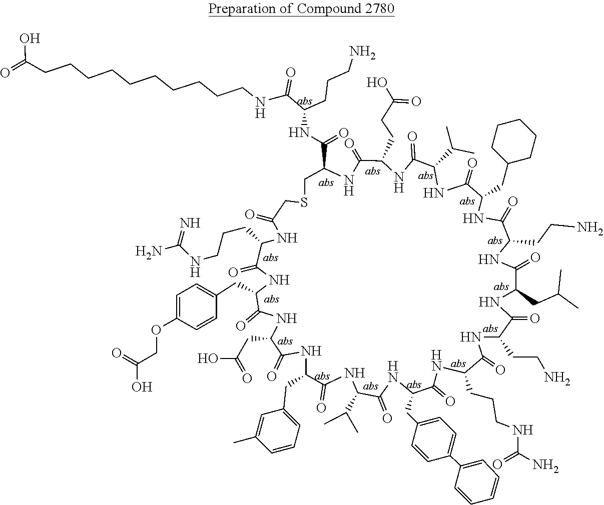

Compound 2780 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5 mg, and its estimated purity by LCMS analysis was 97.4%. Analysis condition A: Retention time=1.58 min; ESI-MS(+) m/z [M+2H]²⁺: 1143.9.

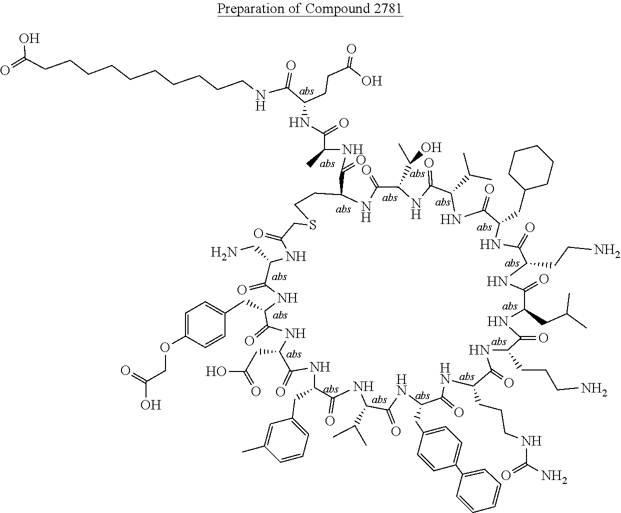

Compound 2781 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 14.7 mg, and its estimated purity by LCMS analysis was 96.1%. Analysis condition A: Retention time=1.54 min; ESI-MS(+) m/z [M+2H]²⁺: 1152.1.

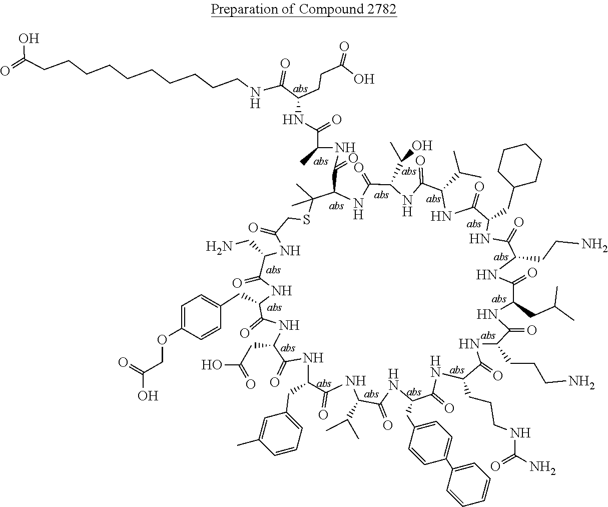

Compound 2782 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 19% B, 19-59% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 49.4 mg, and its estimated purity by LCMS analysis was 94.2%. Analysis condition A: Retention time=1.7, 1.74 min; ESI-MS(+) m/z [M+2H]²⁺: 1159.23, 1159.23.

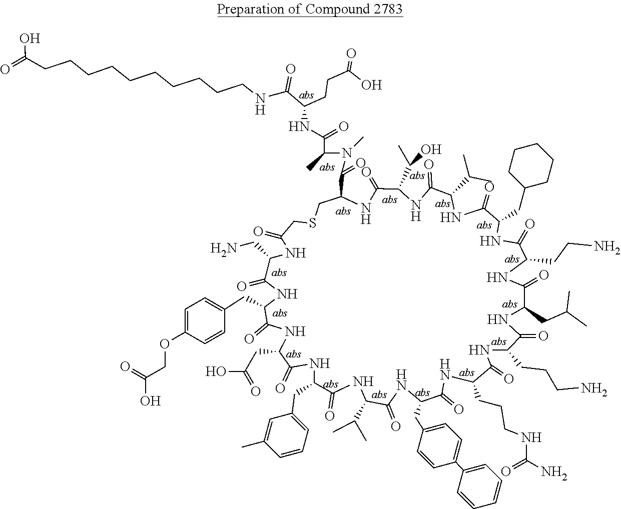

Compound 2783 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 21 mg, and its estimated purity by LCMS analysis was 96.2%. Analysis condition B: Retention time=1.75 min; ESI-MS(+) m/z [M+3H]³⁺: 768.2.

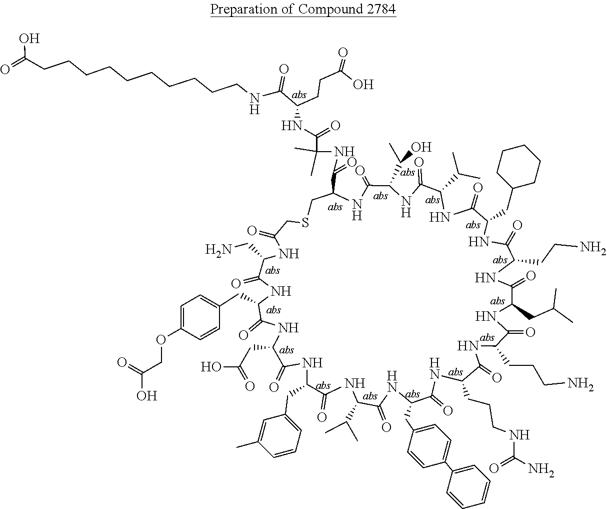

Compound 2784 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 22 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 35.8 mg, and its estimated purity by LCMS analysis was 93.7%. Analysis condition B: Retention time=1.74 min; ESI-MS(+) m/z [M+3H]³⁺: 768.2.

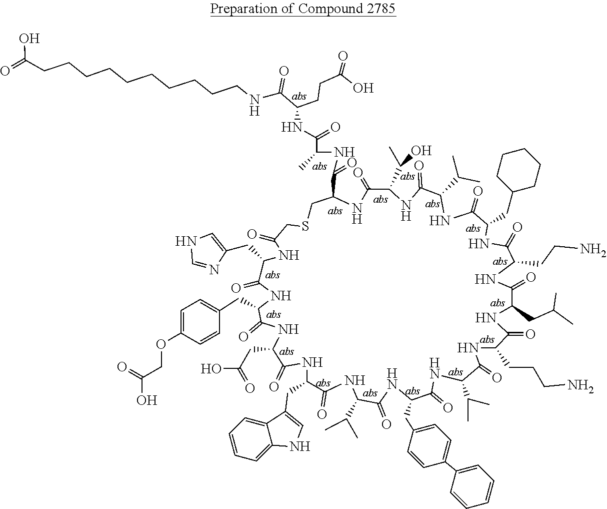 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2c[nH]cn2)NC(=O)CSC[C@@H](C(=O)N[C@H](C)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2785 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.7 mg, and its estimated purity by LCMS analysis was 94%. Analysis condition A: Retention time=1.75 min; ESI-MS(+) m/z [M+2H]²⁺: 1154.1.

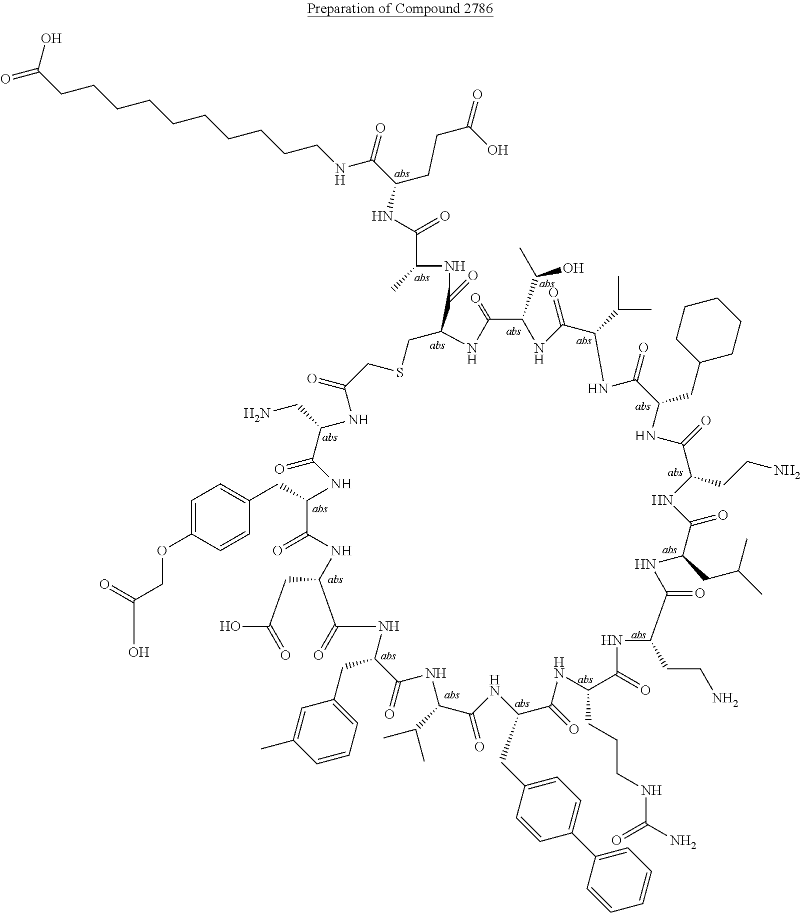 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@H](C)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2786 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.9 mg, and its estimated purity by LCMS analysis was 93%. Analysis condition B: Retention time=1.66 min; ESI-MS(+) m/z [M+2H]²⁺: 1145.2.

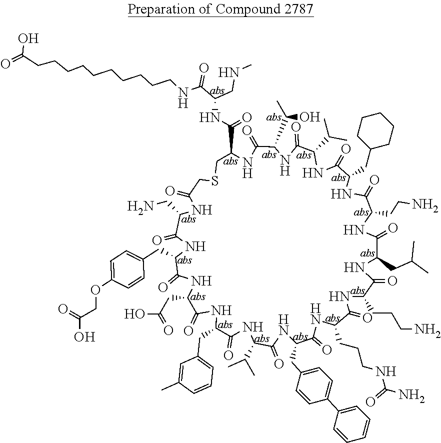 [SMILES: CNC[C@H](NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)NCCCCCCCCCCC(=O)O]

Compound 2787 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 26.3 mg, and its estimated purity by LCMS analysis was 100%. Analysis condition A: Retention time=1.74 min; ESI-MS(+) m/z [M+2H]²⁺: 1095.1.

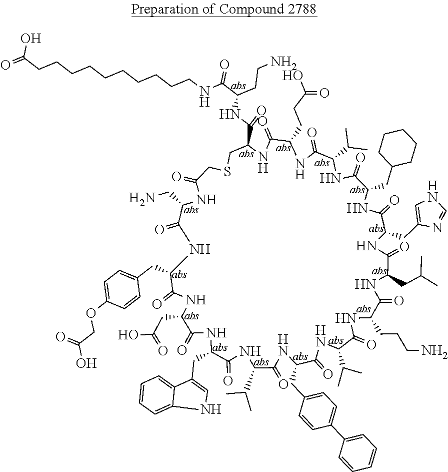 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](Cc2c[nH]cn2)NC1=O]

Compound 2788 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 24.9 mg, and its estimated purity by LCMS analysis was 94.2%. Analysis condition B: Retention time=1.59 min; ESI-MS(+) m/z [M+2H]²⁺: 1111.

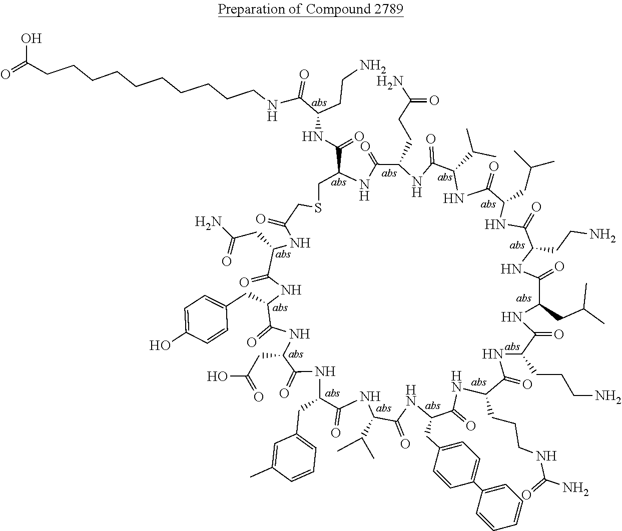 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2789 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.5 mg, and its estimated purity by LCMS analysis was 92.9%. Analysis condition A: Retention time=1.56 min; ESI-MS(+) m/z [M+3H]³⁺: 716.1.

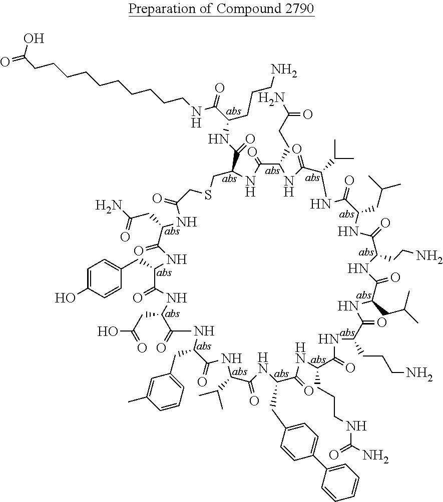 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCCN)C(=O)NCCCCCCCCCCC(=O)O)NC(=O)[C@H](CCC(N)=O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2790 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 13.9 mg, and its estimated purity by LCMS analysis was 97.8%. Analysis condition A: Retention time=1.53, 1.57 min; ESI-MS(+) m/z [M+2H]²⁺: 1080.3, 1080.3.

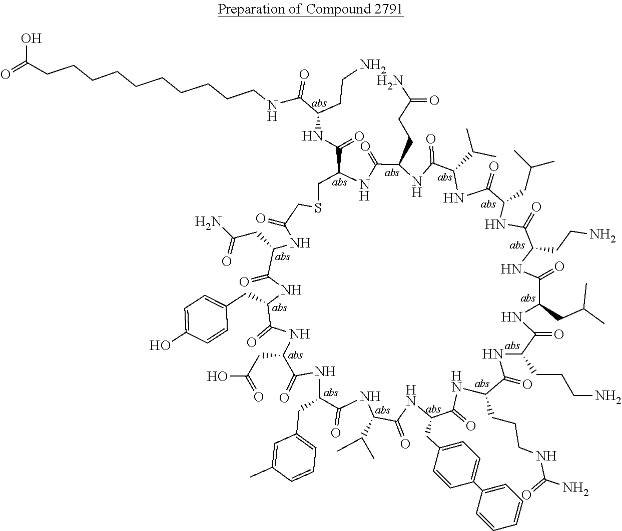 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCN)C(=O)NCCCCCCCCCCC(=O)O)NC(=O)[C@@H](CCC(N)=O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2791 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 40.9 mg, and its estimated purity by LCMS analysis was 99.2%. Analysis condition A: Retention time=1.57 min; ESI-MS(+) m/z [M+3H]³⁺: 716.1.

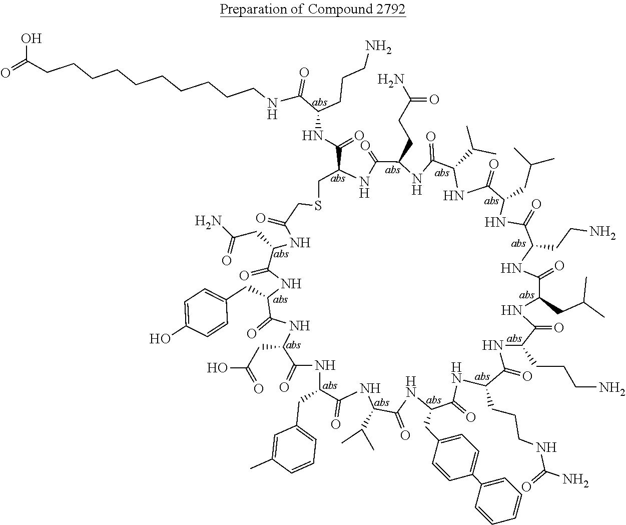

Compound 2792 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 39.7 mg, and its estimated purity by LCMS analysis was 98.7%. Analysis condition A: Retention time=1.56 min; ESI-MS(+) m/z [M+2H]²⁺: 1081.1.

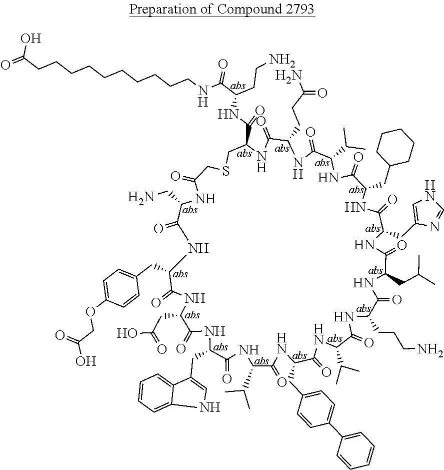 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](Cc2c[nH]cn2)NC1=O]

Compound 2793 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 34.8 mg, and its estimated purity by LCMS analysis was 91.1%. Analysis condition B: Retention time=1.58 min; ESI-MS(+) m/z [M+2H]²⁺: 1110.2.

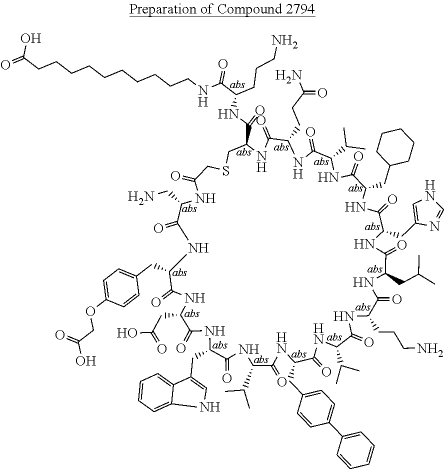 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](Cc2c[nH]cn2)NC1=O]

Compound 2794 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 39.8 mg, and its estimated purity by LCMS analysis was 97.7%. Analysis condition B: Retention time=1.57 min; ESI-MS(+) m/z [M+3H]³⁺: 745.2.

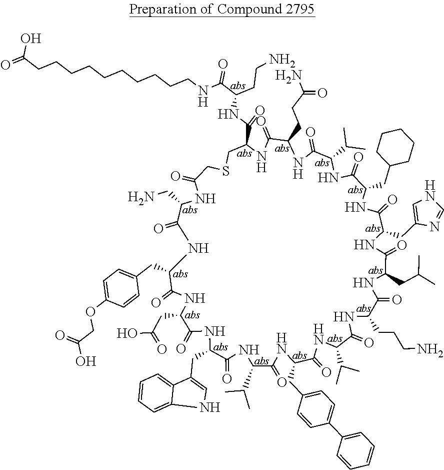 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@@H](CCC(N)=O)NC(=O)[C@H](C(C)C)NC(=O)[C@H](CC2CCCCC2)NC(=O)[C@H](Cc2c[nH]cn2)NC1=O]

Compound 2795 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 23% B, 23-63% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 47 mg, and its estimated purity by LCMS analysis was 99.1%. Analysis condition A: Retention time=1.77 min; ESI-MS(+) m/z [M+3H]³⁺: 741.1.

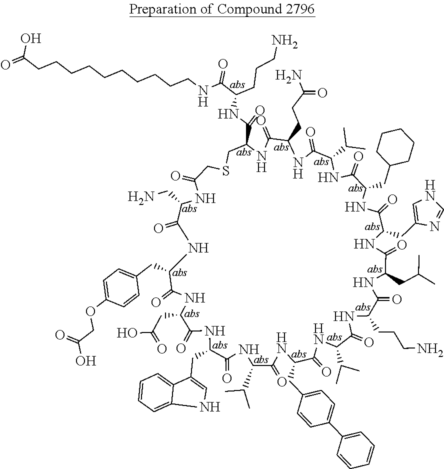 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCCCCCCCCCC(=O)O)NC(=O)[C@@H](CCC(N)=O)NC(=O)[C@H](C(C)C)NC(=O)[C@H](CC2CCCCC2)NC(=O)[C@H](Cc2c[nH]cn2)NC1=O]

Compound 2796 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-65% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 35 mg, and its estimated purity by LCMS analysis was 100%. Analysis condition A: Retention time=1.77 min; ESI-MS(+) m/z [M+2H]²⁺: 1118.2.

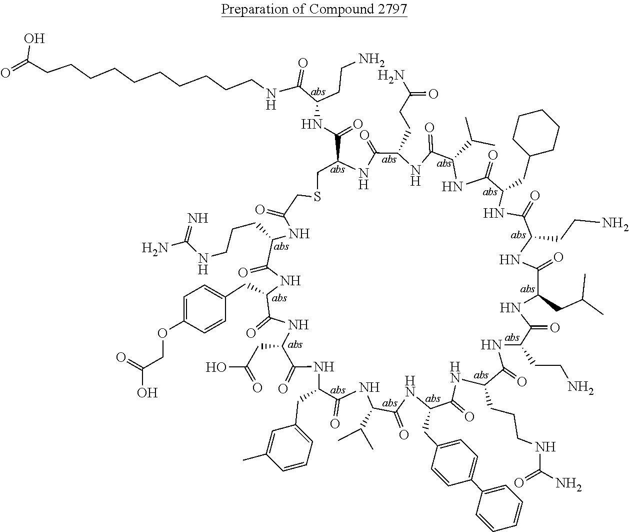

Compound 2797 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.4 mg, and its estimated purity by LCMS analysis was 94.9%. Analysis condition A: Retention time=1.56, 1.59 min; ESI-MS(+) m/z [M+3H]³⁺: 758.16, 7.58.16.

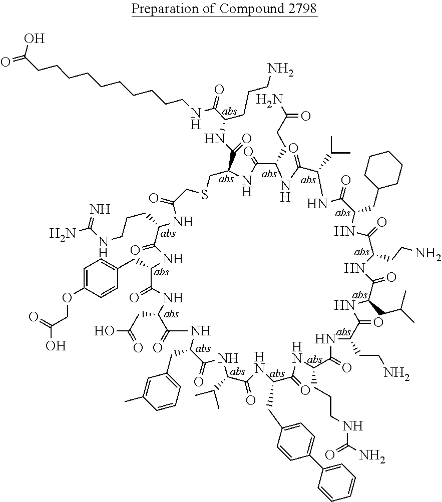 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2798 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 30% B, 30-90% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.2 mg, and its estimated purity by LCMS analysis was 99.2%. Analysis condition B: Retention time=1.57 min; ESI-MS(+) m/z [M+3H]³⁺: 763.1.

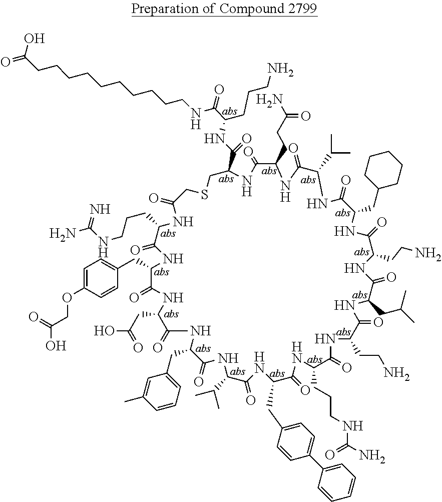 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCN)C(=O)NCCCCCCCCCCC(=O)O)NC(=O)[C@@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2799 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 9.5 mg, and its estimated purity by LCMS analysis was 97.4%. Analysis condition B: Retention time=1.57 min; ESI-MS(+) m/z [M+2H]²⁺: 1143.1.

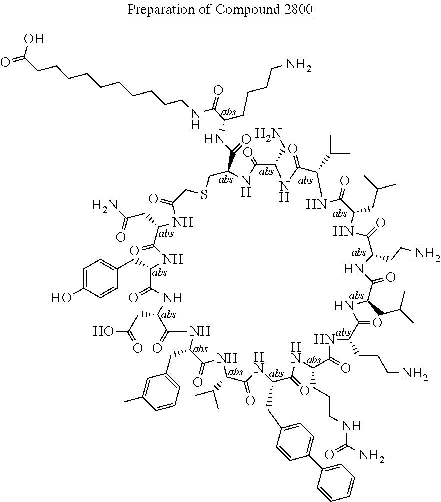 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2800 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 13% B, 13-53% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 3.1 mg, and its estimated purity by LCMS analysis was 100%. Analysis condition A: Retention time=1.58 min; ESI-MS(+) m/z [M+3H]³⁺: 711.2.

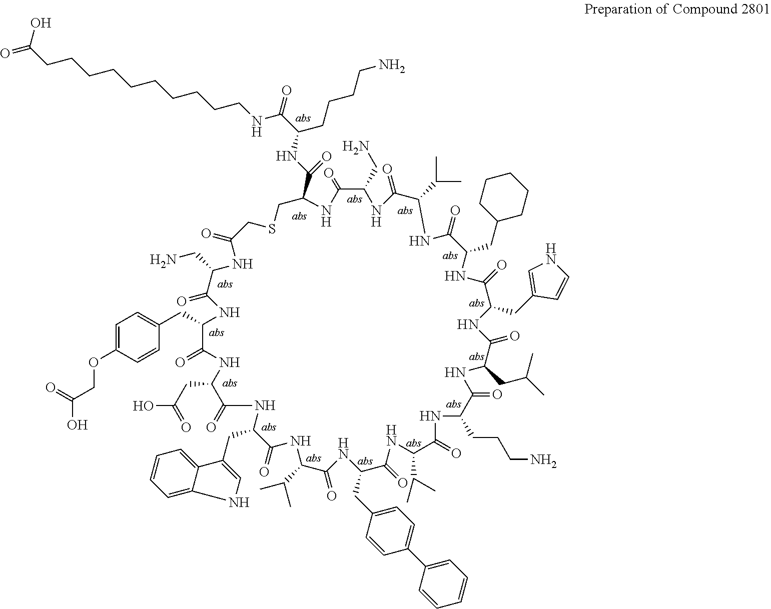 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](Cc2cc[nH]c2)NC1=O]

Compound 2801 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4 mg, and its estimated purity by LCMS analysis was 96.3%. Analysis condition B: Retention time=1.71 min; ESI-MS(+) m/z [M+3H]³⁺: 736.3.

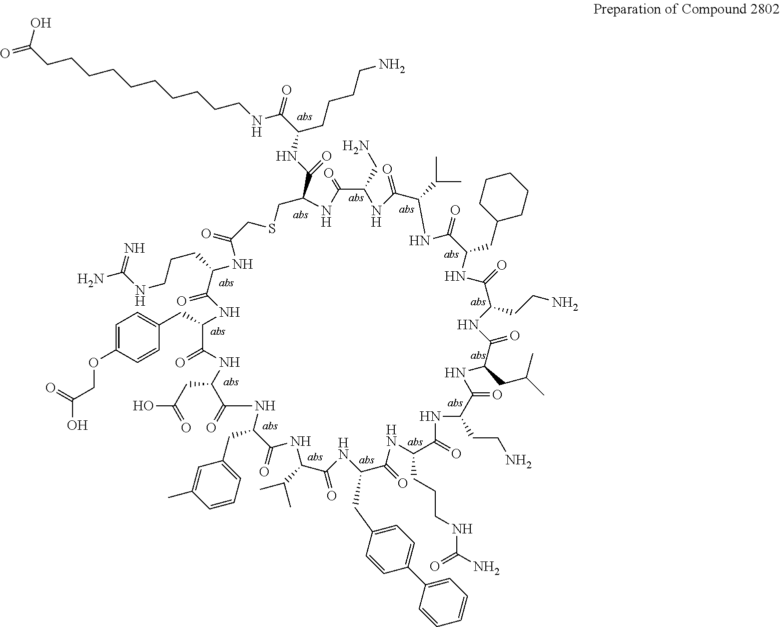 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2802 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.8 mg, and its estimated purity by LCMS analysis was 100%. Analysis condition A: Retention time=1.49 min; ESI-MS(+) m/z [M+3H]³⁺: 753.3.

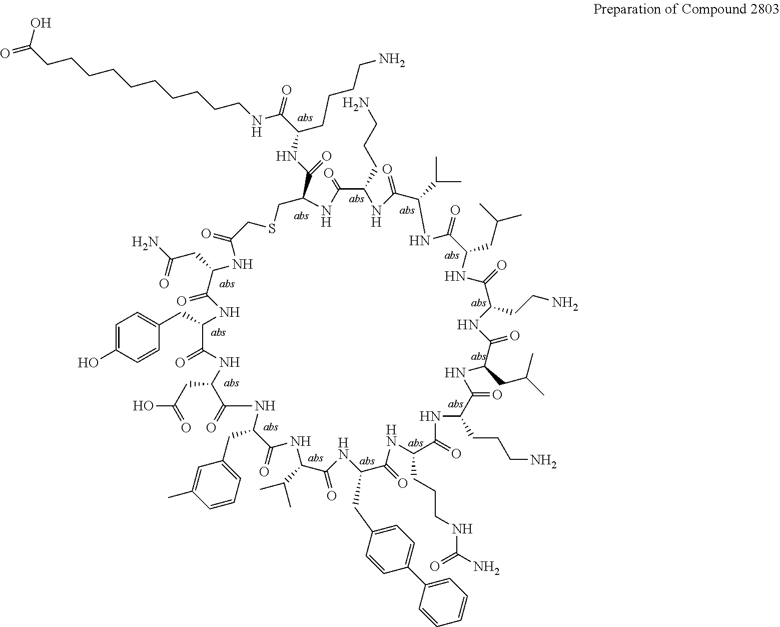 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2803 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 11% B, 11-51% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.6 mg, and its estimated purity by LCMS analysis was 98.1%. Analysis condition A: Retention time=1.51 min; ESI-MS(+) m/z [M+2H]²⁺: 1080.2.

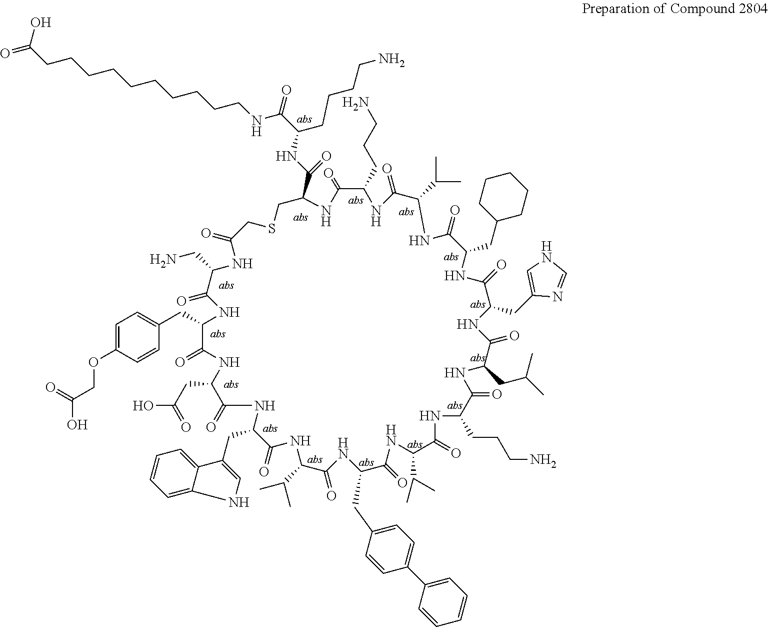 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](Cc2c[nH]cn2)NC1=O]

Compound 2804 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-52% B over 20 minutes, then a 2-minute hold at 100% B; Flow Rate: 45 mL/min; Column Temperature: 25 C. Fraction collection was triggered centrifugal evaporation. The yield of the product was 8.4 mg, and its estimated purity by LCMS analysis was 100%. Analysis condition A: Retention time=1.68 min; ESI-MS(+) m/z [M+3H]³⁺: 745.2.

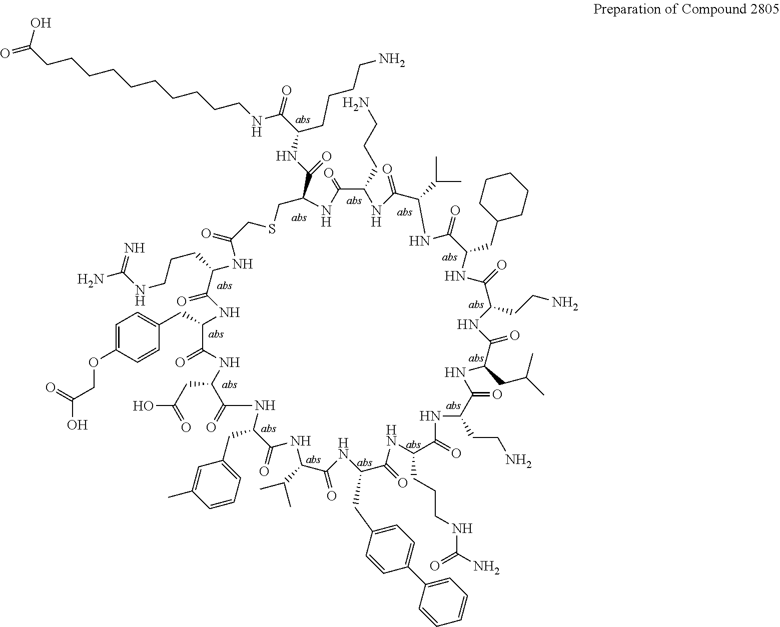 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2805 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 11% B, 11-51% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 12% B, 12-52% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5.9 mg, and its estimated purity by LCMS analysis was 99%. Analysis condition B: Retention time=1.51 min; ESI-MS(+) m/z [M+3H]³⁺: 763.1.

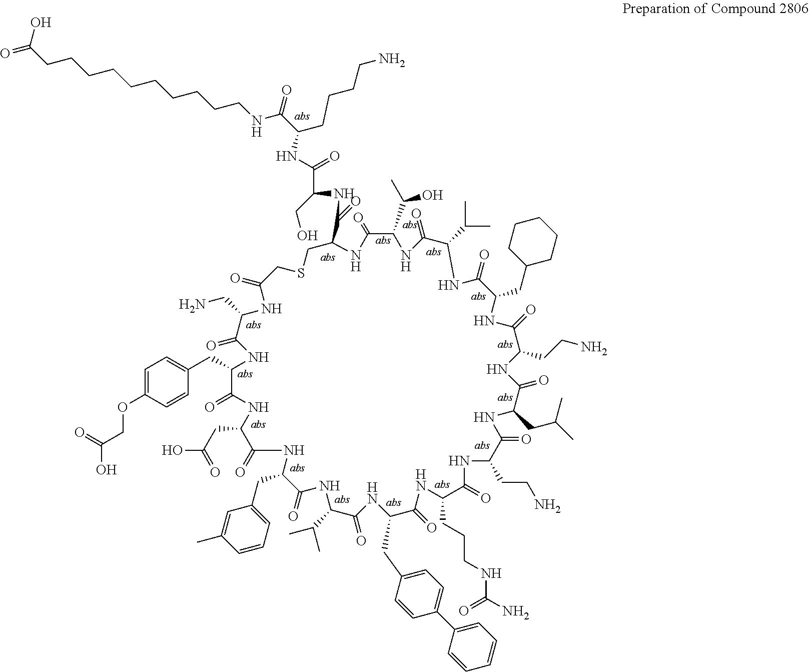 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CO)C(=O)N[C@@H](CCCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2806 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.5 mg, and its estimated purity by LCMS analysis was 90.3%. Analysis condition B: Retention time=1.69 min; ESI-MS(+) m/z [M+2H]²⁺: 1153.

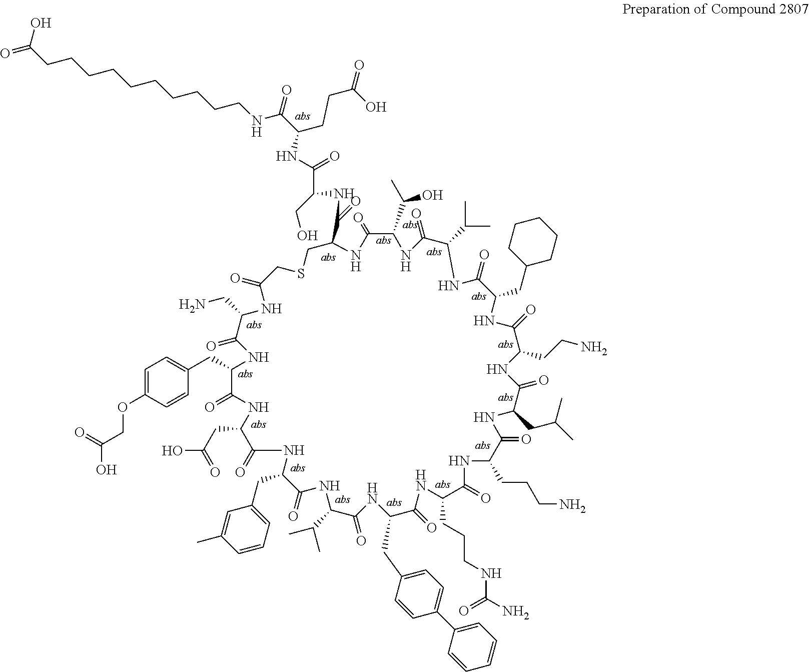 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@H](CO)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2807 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: Waters CSH Fluoro Phenyl, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 2-minute hold at 100% B; Flow Rate: 25 mL/min; Column Temperature: 25 C. Fraction collection was triggered by MS and UV signals. Fractions containing the desired product were combined and dried via centrifugal evaporation. The yield of the product was 4.2 mg, and its estimated purity by LCMS analysis was 100%. Analysis condition A: Retention time=1.62 min; ESI-MS(+) m/z [M+2H]²⁺: 1153.2.

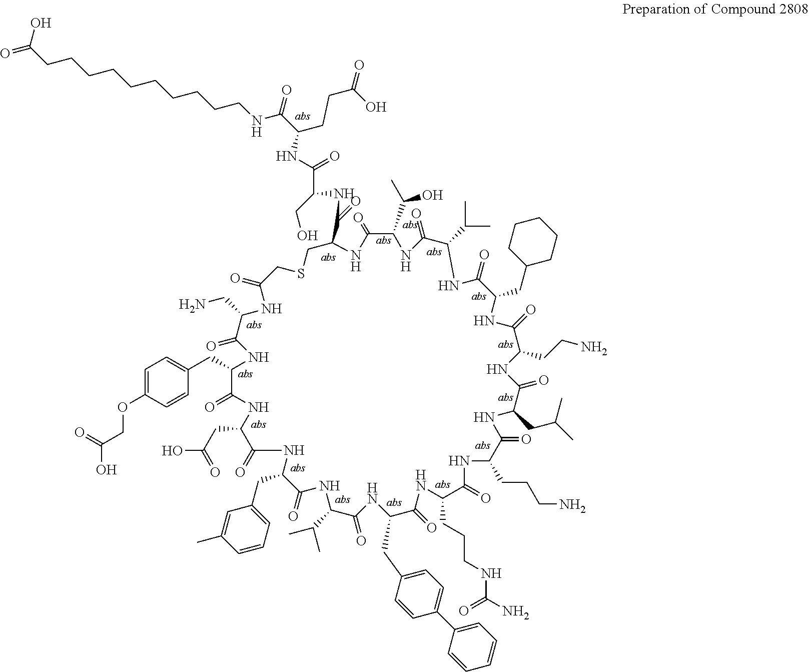 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@H](CO)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2808 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 4 mg, and its estimated purity by LCMS analysis was 100%. Analysis condition A: Retention time=1.64 min; ESI-MS(+) m/z [M+2H]²⁺: 1160.

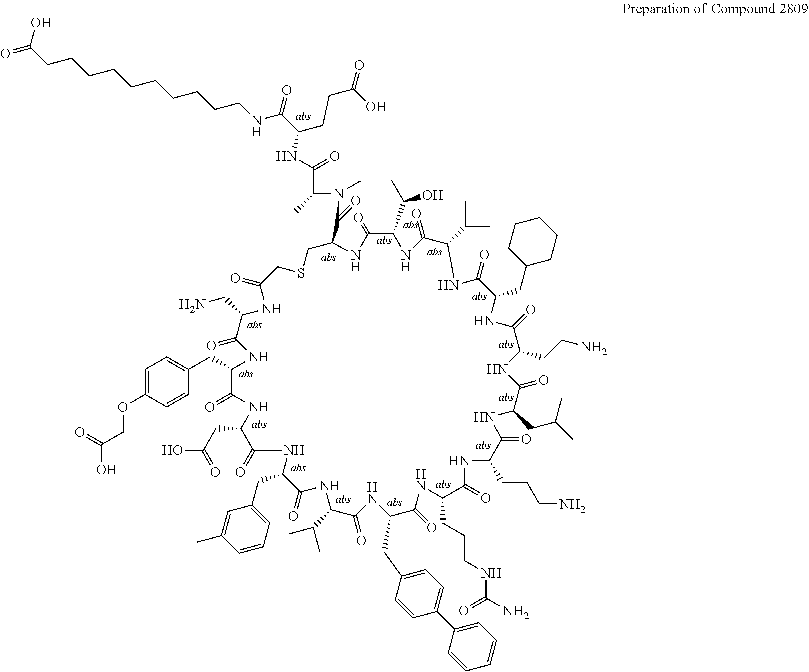 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N(C)[C@H](C)C(=O)N[C@@H](CCC(=O)O)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2809 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.6 mg, and its estimated purity by LCMS analysis was 93%. Analysis condition B: Retention time=1.6 min; ESI-MS(+) m/z [M+2H]²⁺: 1152.2.

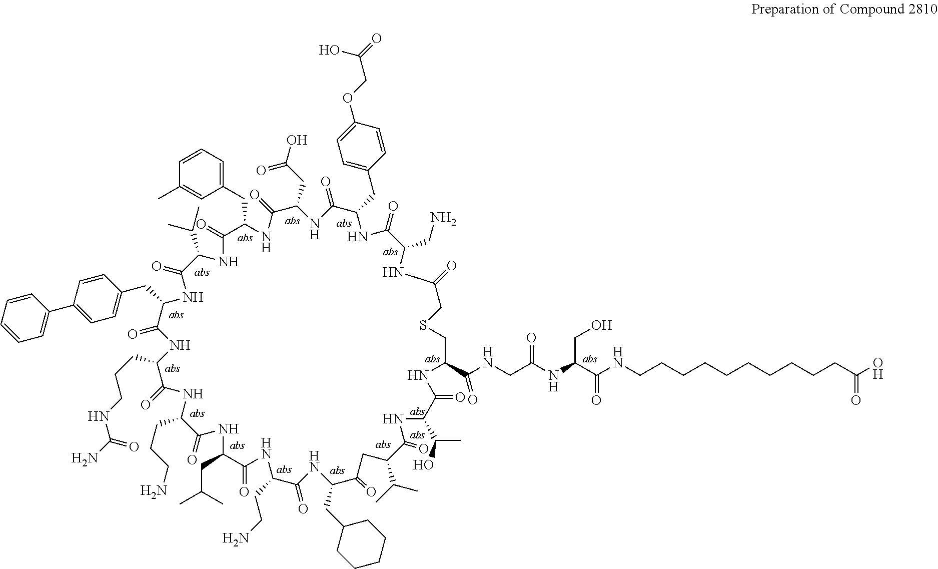 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(=O)N[C@@H](CO)C(=O)NCCCCCCCCCCC(=O)O)NC(=O)[C@H]([C@@H](C)O)NC(=O)[C@H](C(C)C)CC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2810 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.3 mg, and its estimated purity by LCMS analysis was 94.3%. Analysis condition B: Retention time=1.55 min; ESI-MS(+) m/z [M+2H]²⁺: 1117.1.

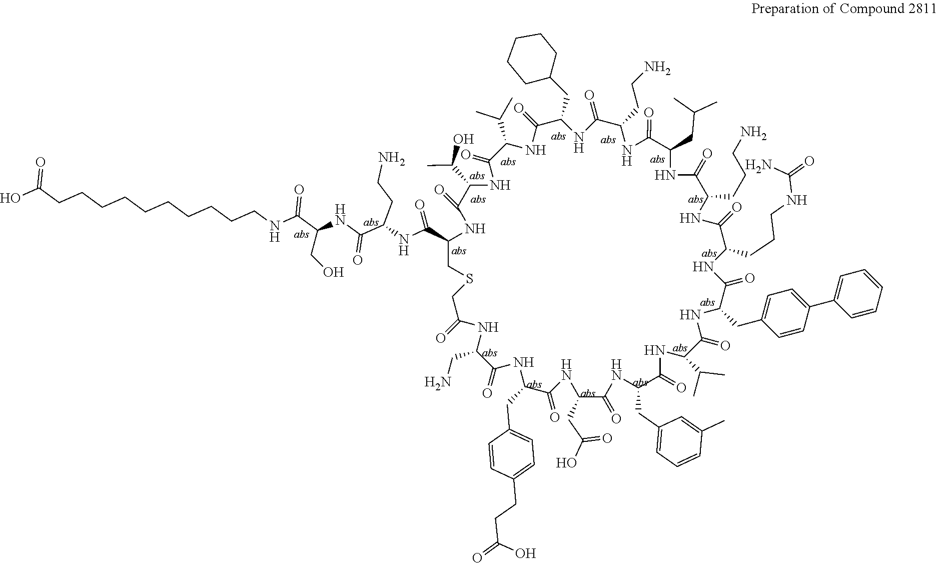 [SMILES: Cc1cccc(C[C@@H]2NC(=O)[C@H](CC(=O)O)NC(=O)[C@H](Cc3ccc(CCC(=O)O)cc3)NC(=O)[C@H](CN)NC(=O)CSC[C@@H](C(=O)N[C@@H](CCN)C(=O)N[C@@H](CO)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2811 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.4 mg, and its estimated purity by LCMS analysis was 93.3%. Analysis condition A: Retention time=1.66 min; ESI-MS(+) m/z [M+3H]³⁺: 759.4.

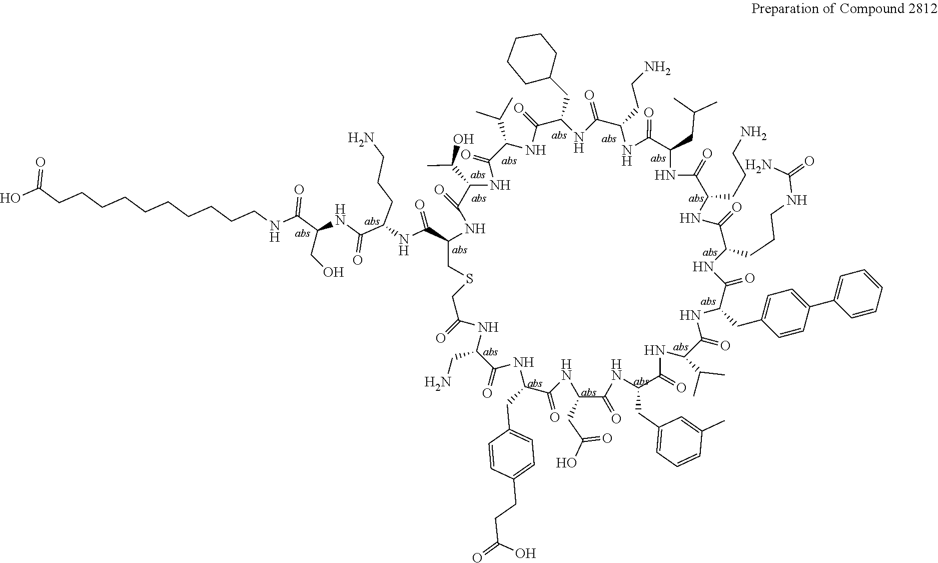 [SMILES: Cc1cccc(C[C@@H]2NC(=O)[C@H](CC(=O)O)NC(=O)[C@H](Cc3ccc(CCC(=O)O)cc3)NC(=O)[C@H](CN)NC(=O)CSC[C@@H](C(=O)N[C@@H](CCCN)C(=O)N[C@@H](CO)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2812 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.3 mg, and its estimated purity by LCMS analysis was 96.9%. Analysis condition B: Retention time=1.62 min; ESI-MS(+) m/z [M+3H]³⁺: 764.2.

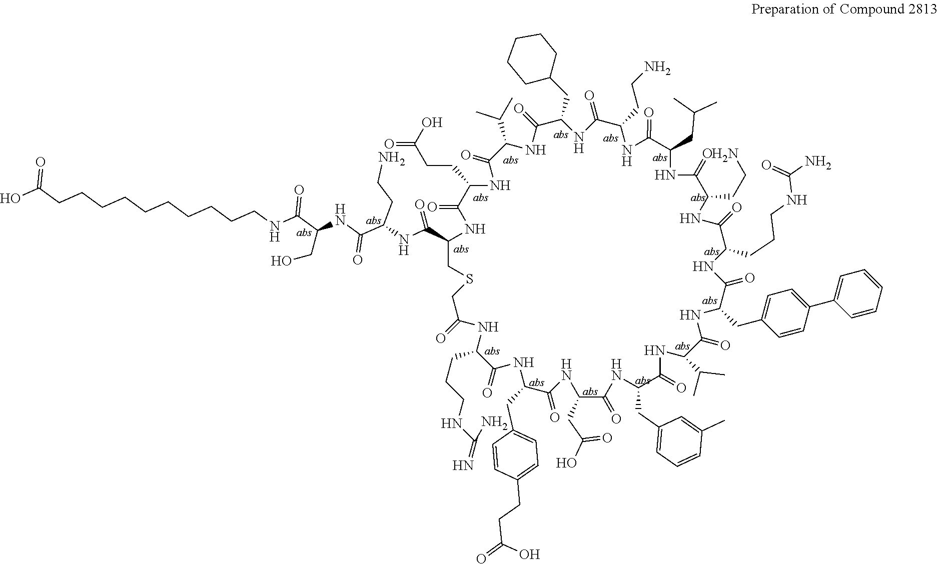 [SMILES: Cc1cccc(C[C@@H]2NC(=O)[C@H](CC(=O)O)NC(=O)[C@H](Cc3ccc(CCC(=O)O)cc3)NC(=O)[C@H](CCCNC(=N)N)NC(=O)CSC[C@@H](C(=O)N[C@@H](CCN)C(=O)N[C@@H](CO)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2813 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×21.2 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.3 mg, and its estimated purity by LCMS analysis was 91.7%. Analysis condition B: Retention time=1.47 min; ESI-MS(+) m/z [M+3H]³⁺: 787.3.

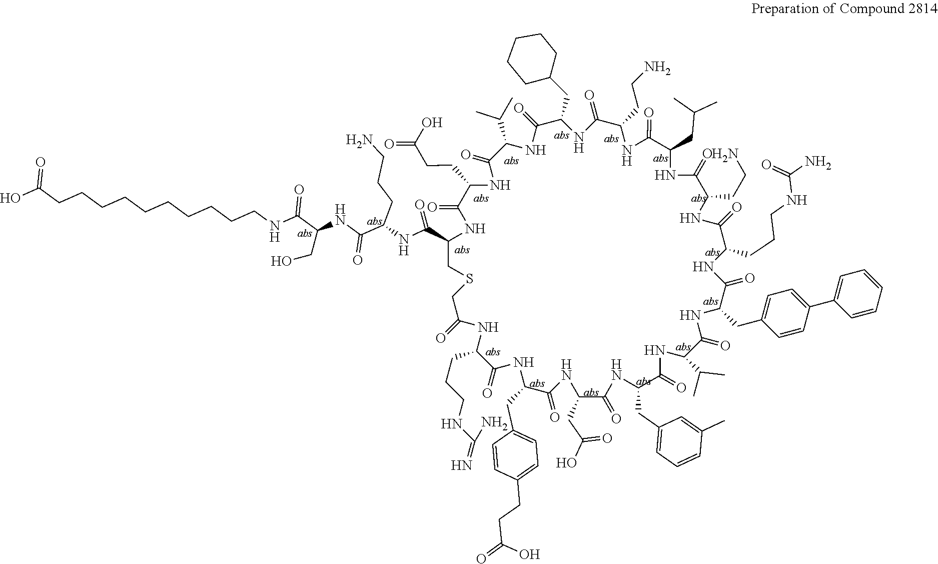 [SMILES: Cc1cccc(C[C@@H]2NC(=O)[C@H](CC(=O)O)NC(=O)[C@H](Cc3ccc(CCC(=O)O)cc3)NC(=O)[C@H](CCCNC(=N)N)NC(=O)CSC[C@@H](C(=O)N[C@@H](CCCN)C(=O)N[C@@H](CO)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2814 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1 mg, and its estimated purity by LCMS analysis was 100%. Analysis condition A: Retention time=1.62 min; ESI-MS(+) m/z [M+3H]³⁺: 792.1.

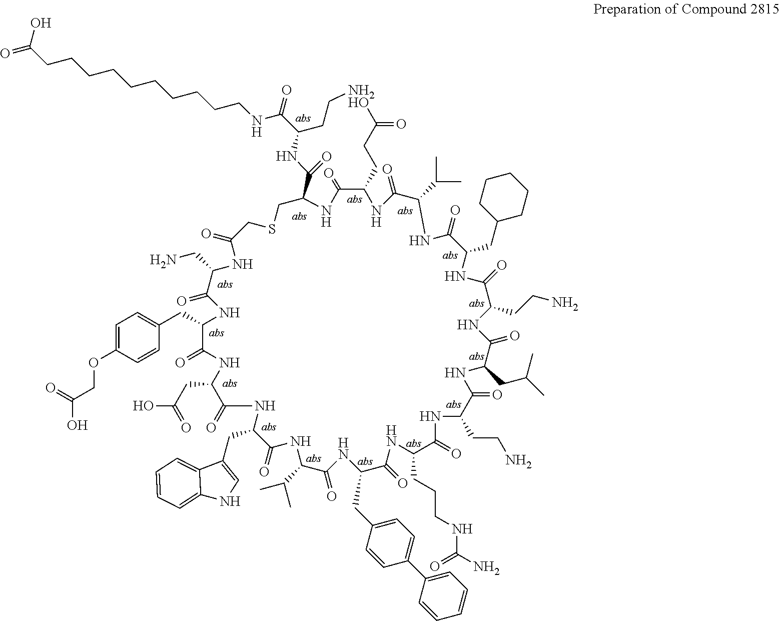 [SMILES: CC(C)C[C@H]1NC(=O)[C@H](CCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 2815 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.9 mg, and its estimated purity by LCMS analysis was 91.4%. Analysis condition B: Retention time=1.56 min; ESI-MS(+) m/z [M+3H]³⁺: 767.2.

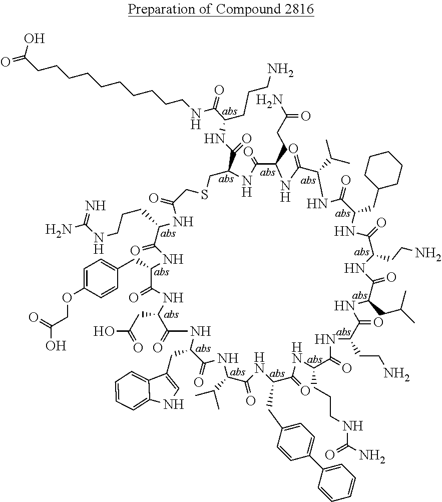 [SMILES: CC(C)C[C@H]1NC(=O)[C@H](CCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCCCCCCCCCC(=O)O)NC(=O)[C@@H](CCC(N)=O)NC(=O)[C@H](C(C)C)NC(=O)[C@H](CC2CCCCC2)NC(=O)[C@H](CCN)NC1=O]

Compound 2816 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.1 mg, and its estimated purity by LCMS analysis was 93.8%. Analysis condition A: Retention time=1.6 min; ESI-MS(+) m/z [M+3H]³⁺: 771.

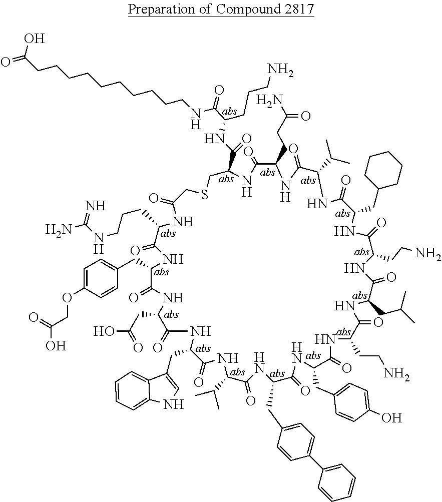 [SMILES: CC(C)C[C@H]1NC(=O)[C@H](CCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCCCCCCCCCC(=O)O)NC(=O)[C@@H](CCC(N)=O)NC(=O)[C@H](C(C)C)NC(=O)[C@H](CC2CCCCC2)NC(=O)[C@H](CCN)NC1=O]

Compound 2817 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 2.1 mg, and its estimated purity by LCMS analysis was 98.1%. Analysis condition A: Retention time=1.64 min; ESI-MS(+) m/z [M+3H]³⁺: 773.1.

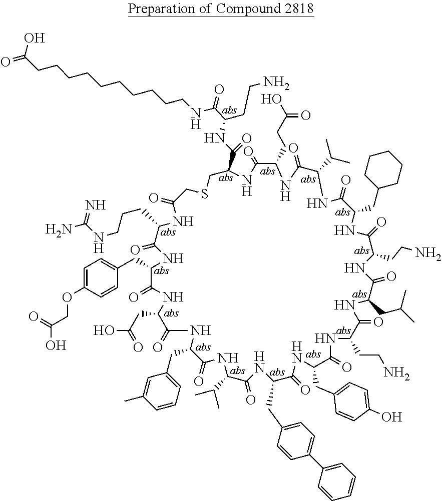 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2818 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.3 mg, and its estimated purity by LCMS analysis was 100%. Analysis condition A: Retention time=1.44, 1.5 min; ESI-MS(+) m/z [M+2H]²⁺: 1140.28, 1140.02.Z

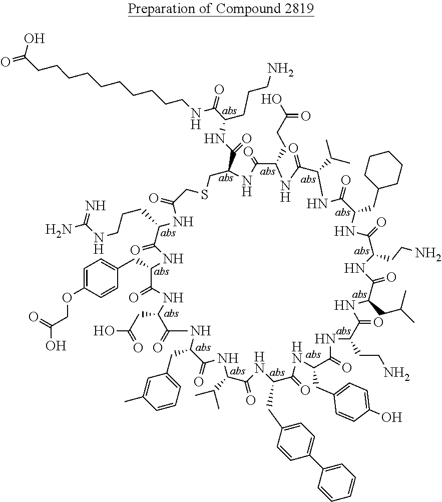 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2819 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.6 mg, and its estimated purity by LCMS analysis was 92%. Analysis condition B: Retention time=1.59 min; ESI-MS(+) m/z [M+3H]³⁺: 765.3.

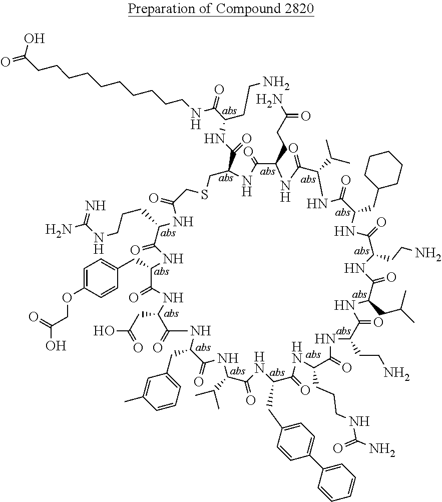 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 2820 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.1 mg, and its estimated purity by LCMS analysis was 95.8%. Analysis condition B: Retention time=1.59 min; ESI-MS(+) m/z [M+2H]²⁺: 1136.4.

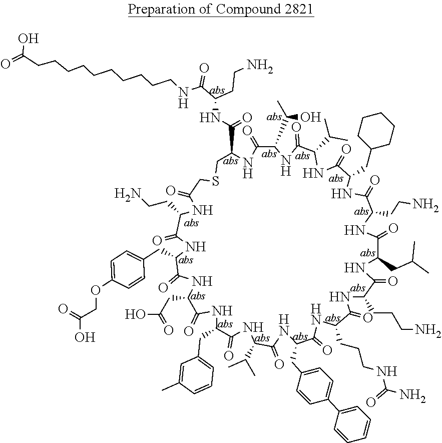 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2821 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: Waters CSH Fluoro Phenyl, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 2-minute hold at 100% B; Flow Rate: 25 mL/min; Column Temperature: 25 C. Fraction collection was triggered by MS and UV signals. Fractions containing the desired product were combined and dried via centrifugal evaporation. The yield of the product was 7.5 mg, and its estimated purity by LCMS analysis was 93.5%. Analysis condition B: Retention time=1.61 min; ESI-MS(+) m/z [M+2H]²⁺: 1101.6.

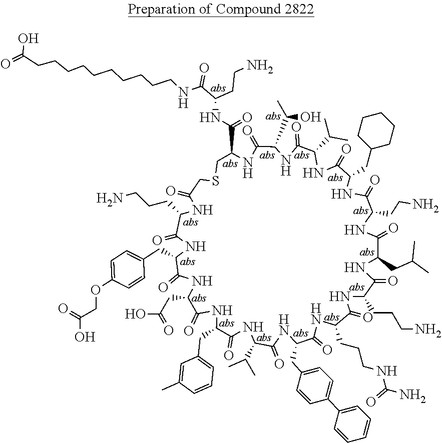 [SMILES: Cc1cccc(C[C@@H]2NC(=O)[C@H](CC(=O)O)NC(=O)[C@H](Cc3ccc(OCC(=O)O)cc3)NC(=O)[C@H](CCCN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2822 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.1 mg, and its estimated purity by LCMS analysis was 95%. Analysis condition B: Retention time=1.62 min; ESI-MS(+) m/z [M+2H]²⁺: 1109.2.

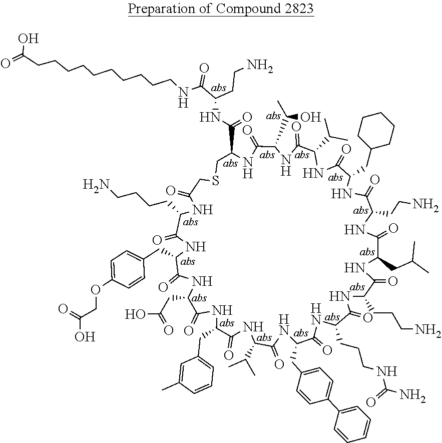 [SMILES: Cc1cccc(C[C@@H]2NC(=O)[C@H](CC(=O)O)NC(=O)[C@H](Cc3ccc(OCC(=O)O)cc3)NC(=O)[C@H](CCCCN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2823 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 6.6 mg, and its estimated purity by LCMS analysis was 92.3%. Analysis condition A: Retention time=1.62 min; ESI-MS(+) m/z [M+3H]³⁺: 744.

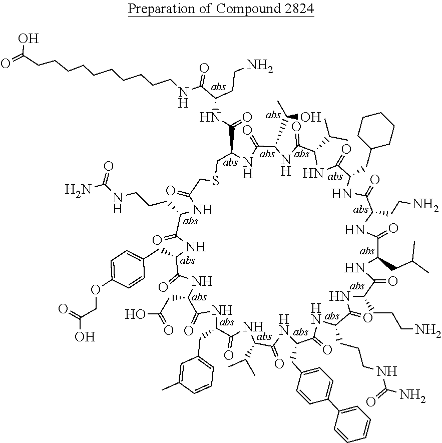 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(N)=O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2824 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 4.7 mg, and its estimated purity by LCMS analysis was 90.7%. Analysis condition B: Retention time=1.7 min; ESI-MS(+) m/z [M+3H]³⁺: 753.9.

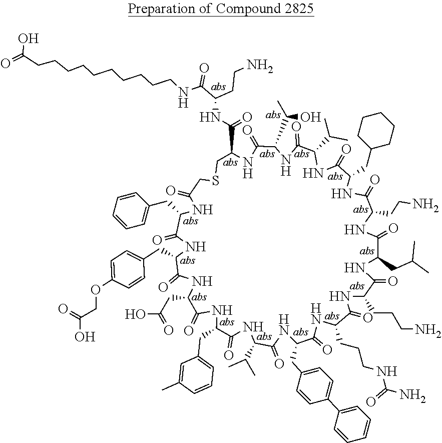 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccccc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2825 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 22% B, 22-62% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 2.9 mg, and its estimated purity by LCMS analysis was 100%. Analysis condition A: Retention time=1.8 min; ESI-MS(+) m/z [M+2H]²⁺: 1125.3.

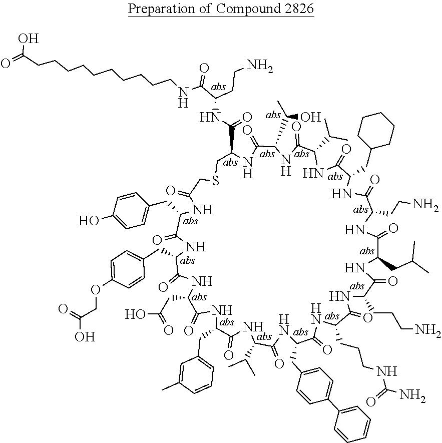 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2826 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 3.2 mg, and its estimated purity by LCMS analysis was 96%. Analysis condition B: Retention time=1.69 min; ESI-MS(+) m/z [M+3H]³⁺: 756.2.

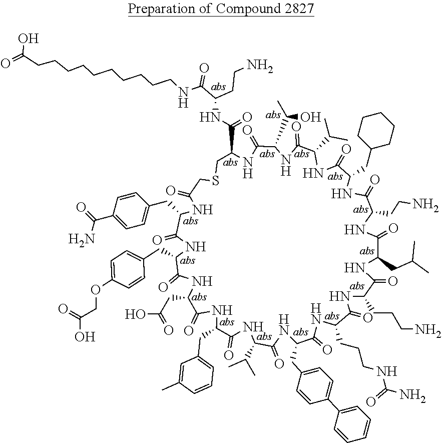 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2827 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.8 mg, and its estimated purity by LCMS analysis was 90.9%. Analysis condition A: Retention time=1.63 min; ESI-MS(+) m/z [M+2H]²⁺: 1147.1.

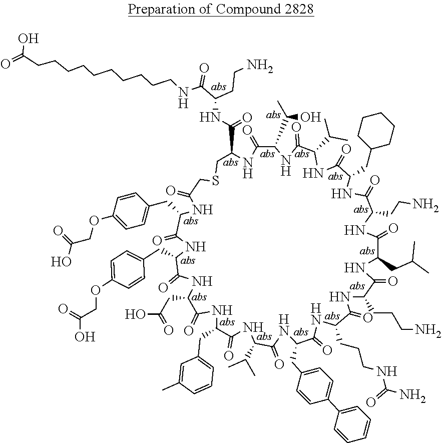 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2828 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 18% B, 18-58% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.9 mg, and its estimated purity by LCMS analysis was 97.3%. Analysis condition B: Retention time=1.73 min; ESI-MS(+) m/z [M+3H]³⁺: 775.2.

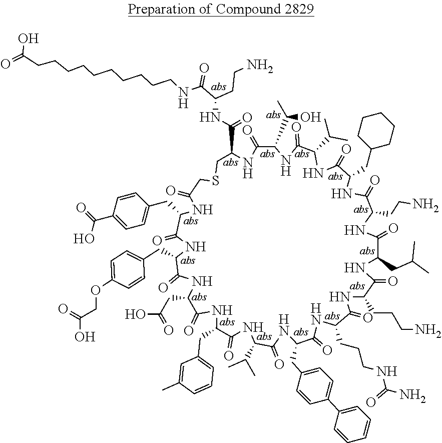 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(=O)O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2829 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.4 mg, and its estimated purity by LCMS analysis was 92.2%. Analysis condition B: Retention time=1.72 min; ESI-MS(+) m/z [M+3H]³⁺: 765.2.

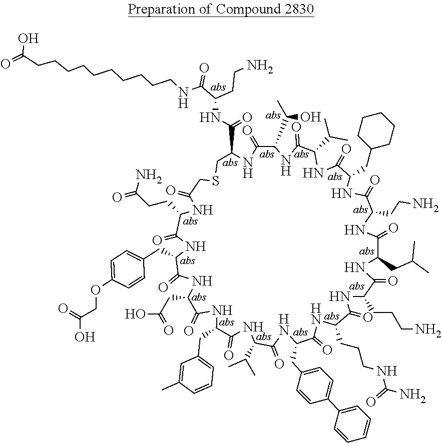 [SMILES: Cc1cccc(C[C@@H]2NC(=O)[C@H](CC(=O)O)NC(=O)[C@H](Cc3ccc(OCC(=O)O)cc3)NC(=O)[C@H](CCC(N)=O)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2830 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.2 mg, and its estimated purity by LCMS analysis was 97.5%. Analysis condition B: Retention time=1.71 min; ESI-MS(+) m/z [M+2H]²⁺: 1116.2.

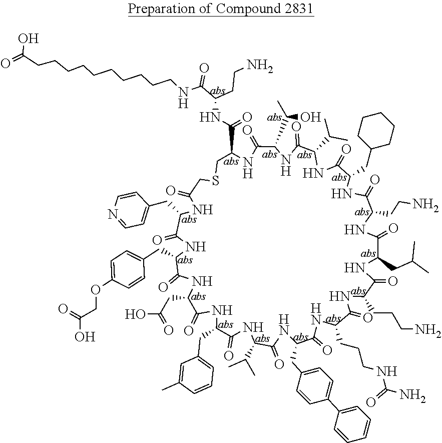 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2831 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 4.4 mg, and its estimated purity by LCMS analysis was 92.6%. Analysis condition A: Retention time=1.67 min; ESI-MS(+) m/z [M+3H]³⁺: 751.2.

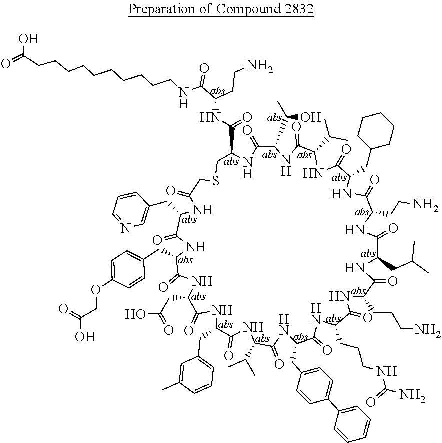 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2832 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.8 mg, and its estimated purity by LCMS analysis was 95.9%. Analysis condition A: Retention time=1.64 min; ESI-MS(+) m/z [M+2H]²⁺: 1126.3.

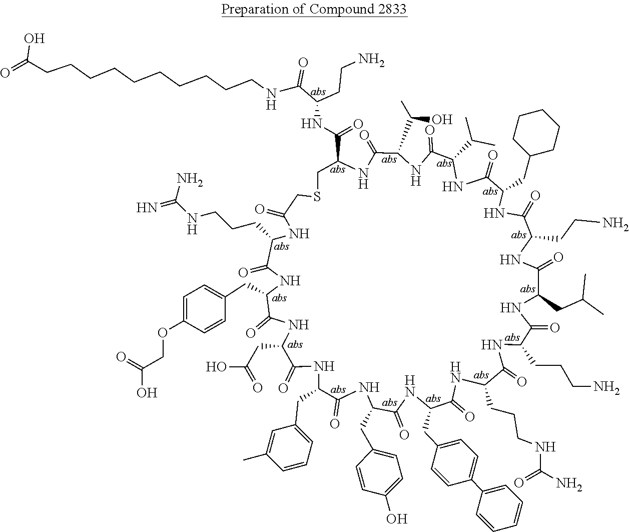

Compound 2833 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.9 mg, and its estimated purity by LCMS analysis was 92.5%. Analysis condition A: Retention time=1.63 min; ESI-MS(+) m/z [M+2H]²⁺: 1162.2.

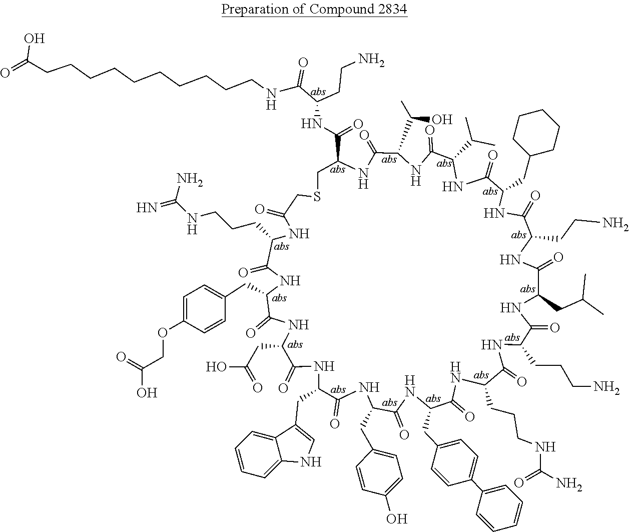

Compound 2834 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 11.9 mg, and its estimated purity by LCMS analysis was 96.5%. Analysis condition A: Retention time=1.41 min; ESI-MS(+) m/z [M+3H]³⁺: 783.2.

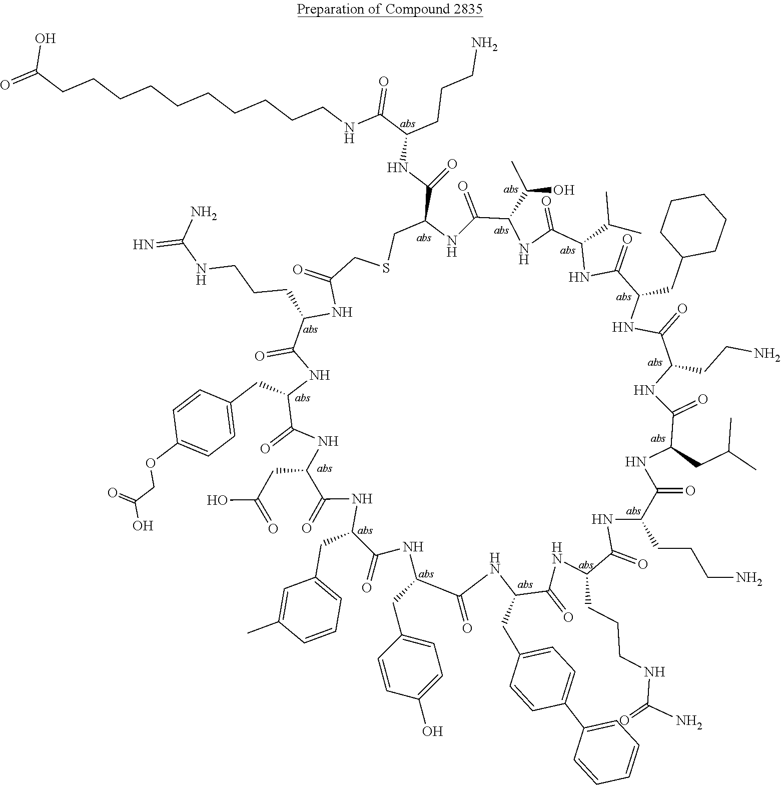 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(O)cc3)NC2=O)c1]

Compound 2835 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.2 mg, and its estimated purity by LCMS analysis was 93.6%. Analysis condition B: Retention time=1.62 min; ESI-MS(+) m/z [M+2H]²⁺: 1169.2.

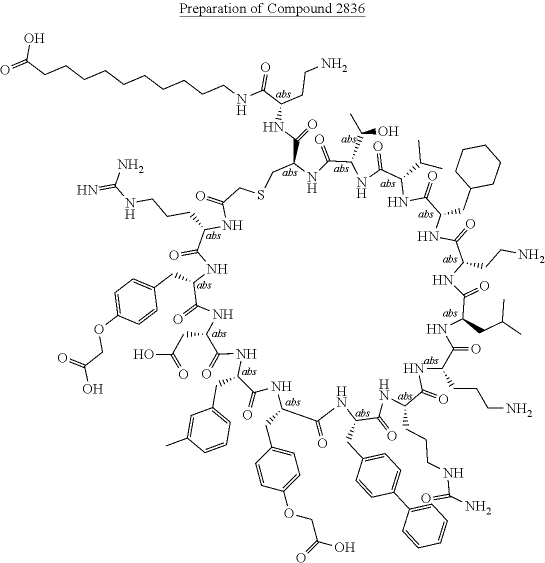 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(OCC(=O)O)cc3)NC2=O)c1]

Compound 2836 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 1.8 mg, and its estimated purity by LCMS analysis was 98.8%. Analysis condition A: Retention time=1.61 min; ESI-MS(+) m/z [M+3H]³⁺: 794.1.

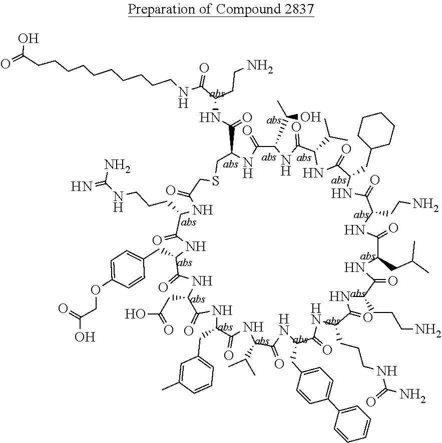 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2837 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS and UV signals. Fractions containing the desired product were combined and dried via centrifugal evaporation. The yield of the product was 12.9 mg, and its estimated purity by LCMS analysis was 91.4%. Analysis condition A: Retention time=1.69 min; ESI-MS(+) m/z [M+3H]³⁺: 758.2.

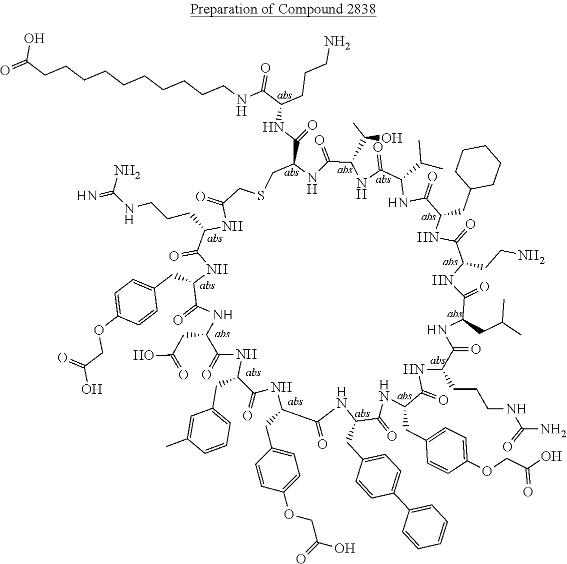 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2838 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 21% B, 21-61% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 2-minute hold at centrifugal evaporation. The yield of the product was 1.5 mg, and its estimated purity by LCMS analysis was 96.8%. Analysis condition A: Retention time=1.62 min; ESI-MS(+) m/z [M+3H]³⁺: 834.2.

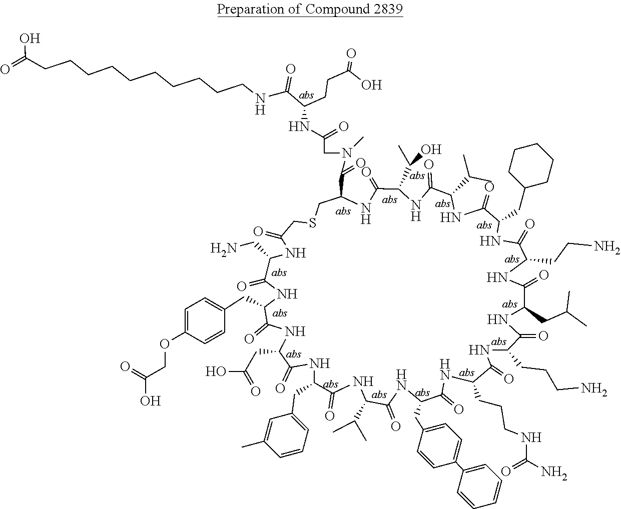

Compound 2839 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 16% B, 16-56% B over 23 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: Waters CSH Fluoro Phenyl, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 2-minute hold at 100% B; Flow Rate: 25 mL/min; Column Temperature: 25 C. Fraction collection was triggered by MS and UV signals. Fractions containing the desired product were combined and dried via centrifugal evaporation. The yield of the product was 5.2 mg, and its estimated purity by LCMS analysis was 91.5%. Analysis condition B: Retention time=1.71 min; ESI-MS(+) m/z [M+2H]²⁺: 1145.2.

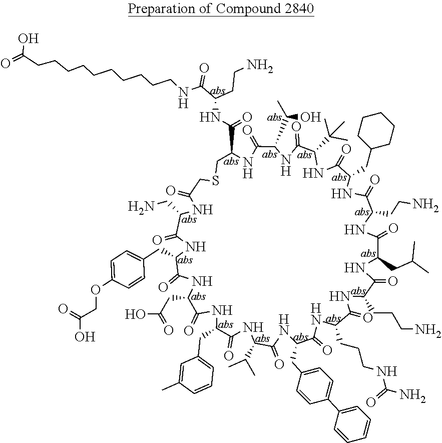 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2840 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 17% B, 17-57% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.9 mg, and its estimated purity by LCMS analysis was 98.9%. Analysis condition A: Retention time=1.51 min; ESI-MS(+) m/z [M+3H]³⁺: 735.1.

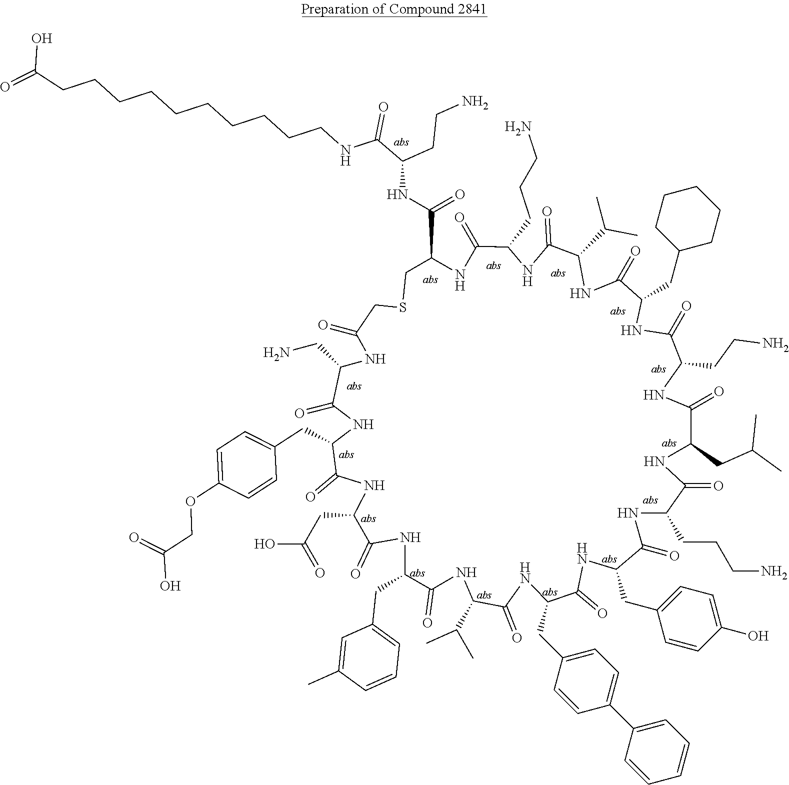 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2841 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.1 mg, and its estimated purity by LCMS analysis was 90.4%. Analysis condition B: Retention time=1.48 min; ESI-MS(+) m/z [M+2H]²⁺: 1104.3.

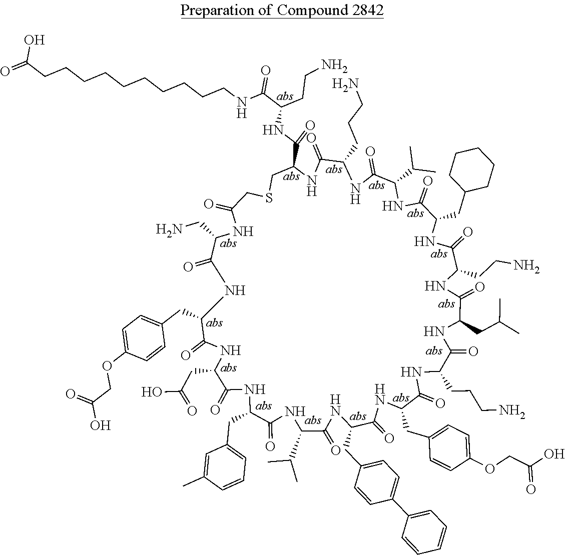 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 2842 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 2 mg, and its estimated purity by LCMS analysis was 100%. Analysis condition A: Retention time=1.49 min; ESI-MS(+) m/z [M+3H]³⁺: 756.1.

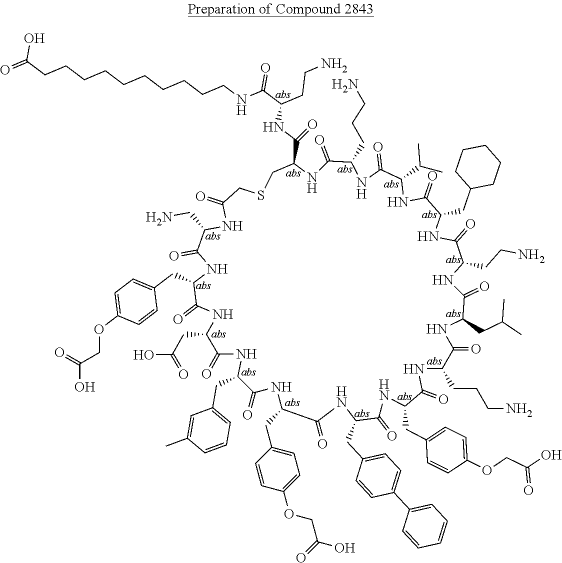 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2843 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 2-minute hold at 100% B; Flow Rate: 45 mL/min; Column Temperature: 25 C. Fraction collection was triggered centrifugal evaporation. The yield of the product was 0.7 mg, and its estimated purity by LCMS analysis was 100%. Analysis condition A: Retention time=1.36 min; ESI-MS(+) m/z [M+2H]²⁺: 1195.1.

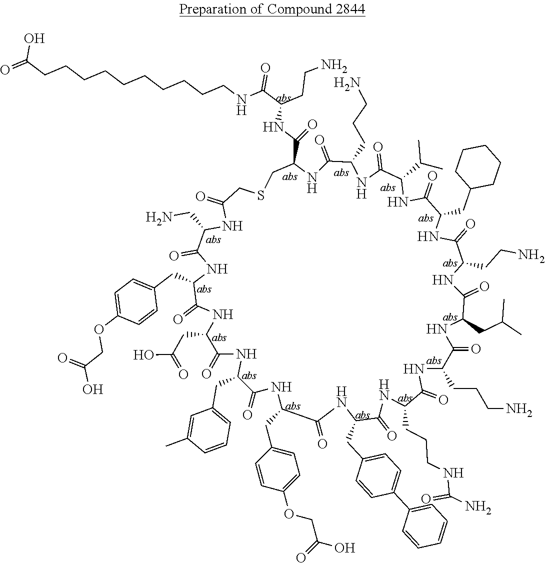 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(OCC(=O)O)cc3)NC2=O)c1]

Compound 2844 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 7.9 mg, and its estimated purity by LCMS analysis was 100%. Analysis condition A: Retention time=1.48 min; ESI-MS(+) m/z [M+3H]³⁺: 775.2.

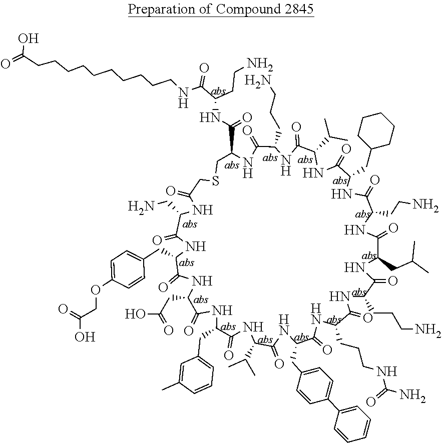 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2845 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 25 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.6 mg, and its estimated purity by LCMS analysis was 100%. Analysis condition A: Retention time=1.52 min; ESI-MS(+) m/z [M+3H]³⁺: 735.

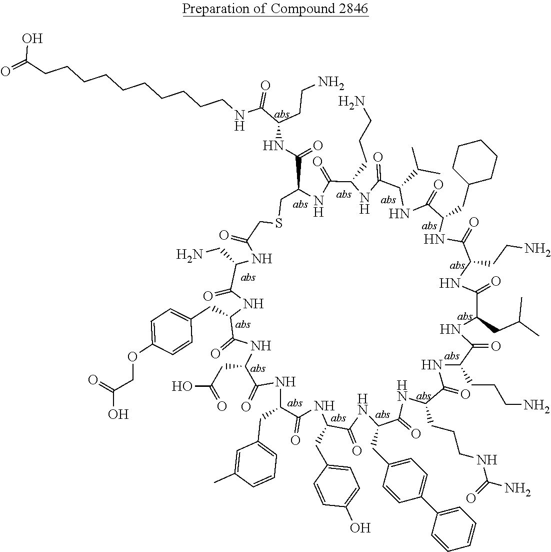 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(O)cc3)NC2=O)c1]

Compound 2846 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17 mg, and its estimated purity by LCMS analysis was 97%. Analysis condition A: Retention time=1.53 min; ESI-MS(+) m/z [M+3H]³⁺: 756.3.

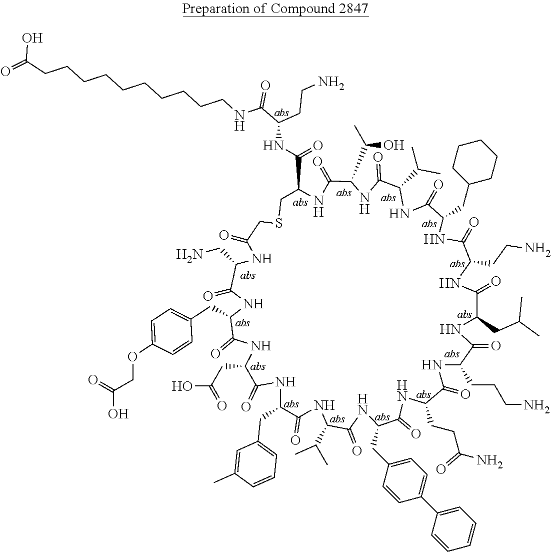 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 2847 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Example 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14 mg, and its estimated purity by LCMS analysis was 98%. Analysis condition A: Retention time=1.61 min; ESI-MS(+) m/z [M+3H]³⁺: 721.1.

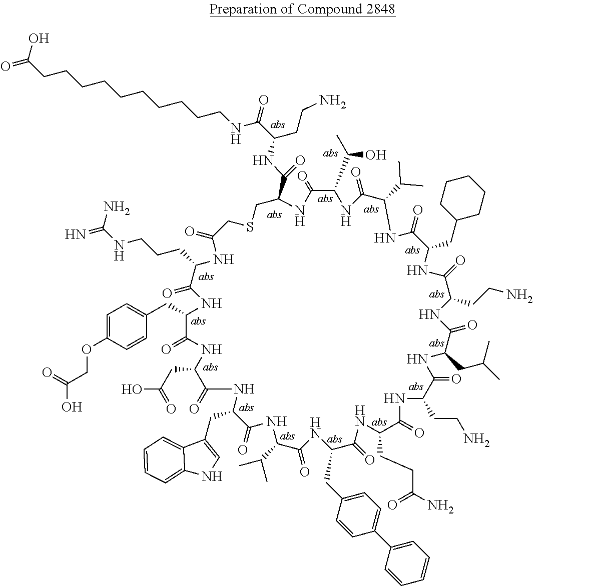 [SMILES: CC(C)C[C@H]1NC(=O)[C@H](CCN)NC(=O)[C@H](CCC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC2CCCCC2)NC(=O)[C@H](CCN)NC1=O]

Compound 2848 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 15.3 mg, and its estimated purity by LCMS analysis was 100%. Analysis condition A: Retention time=1.54 min; ESI-MS(+) m/z [M+3H]³⁺: 752.1.

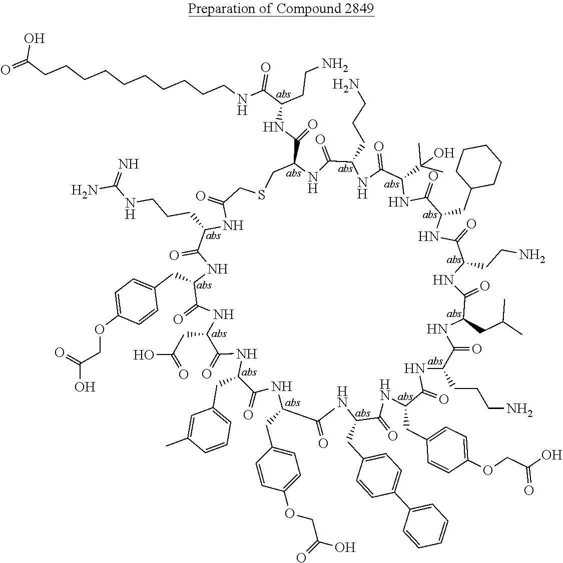 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCN)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2849 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 5.3 mg, and its estimated purity by LCMS analysis was 100%. Analysis condition B: Retention time=1.57 min; ESI-MS(+) m/z [M+3H]³⁺: 825.4.

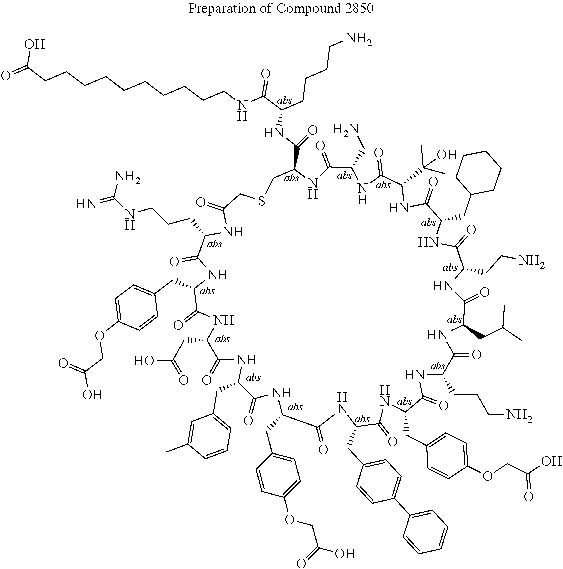 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](CN)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2850 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 4.7 mg, and its estimated purity by LCMS analysis was 98.7%. Analysis condition B: Retention time=1.58 min; ESI-MS(+) m/z [M+3H]³⁺: 825.

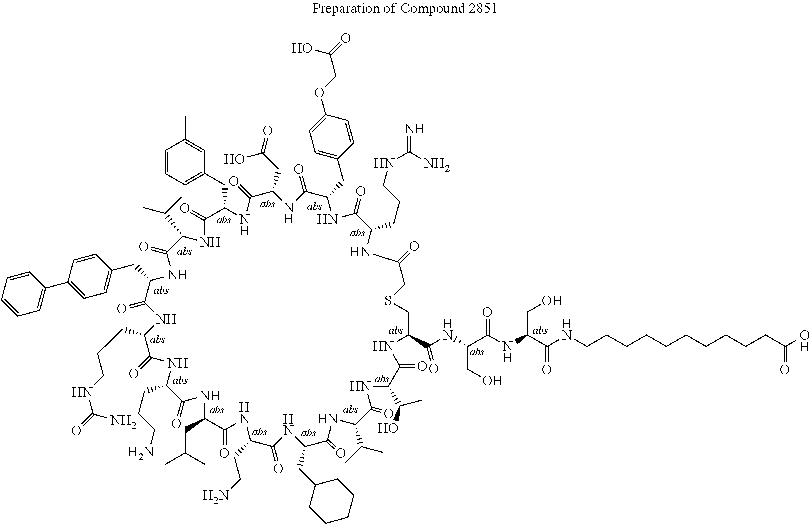 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CO)C(=O)N[C@@H](CO)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2851 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 20% B, 20-60% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 2.9 mg, and its estimated purity by LCMS analysis was 100%. Analysis condition 1: Retention time=1.6 min; ESI-MS(+) m/z [M+3H]³⁺: 778.4.

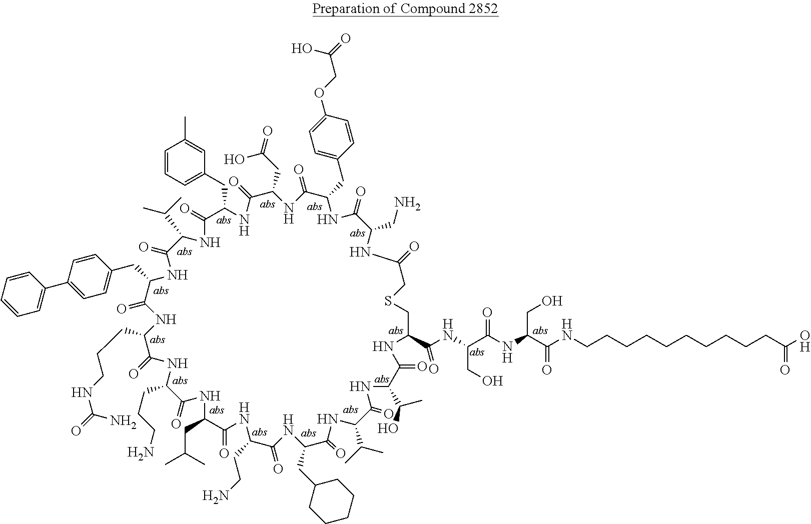 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CO)C(=O)N[C@@H](CO)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2852 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 22% B, 22-58% B over 22 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 1.3 mg, and its estimated purity by LCMS analysis was 90.4%. Analysis condition 1: Retention time=1.63 min; ESI-MS(+) m/z [M+2H]²⁺: 1132.1.

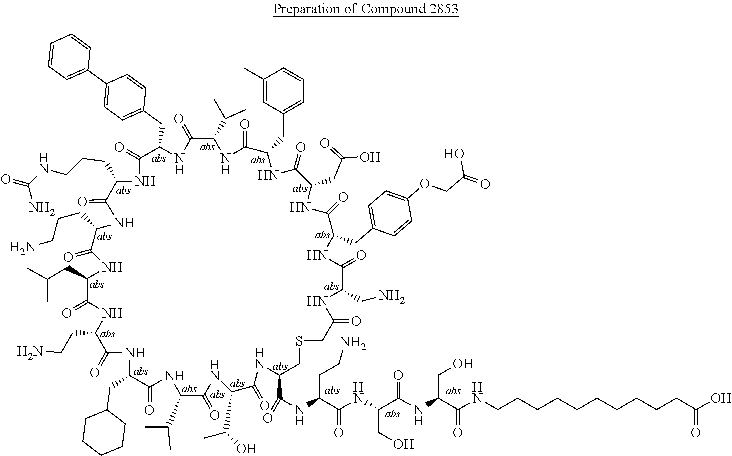 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)N[C@@H](CO)C(=O)N[C@@H](CO)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2853 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 18% B, 18-58% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 17% B, 17-57% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 0.8 mg, and its estimated purity by LCMS analysis was 100%. Analysis condition 1: Retention time=1.51 min; ESI-MS(+) m/z [M+2H]²⁺: 1181.5.

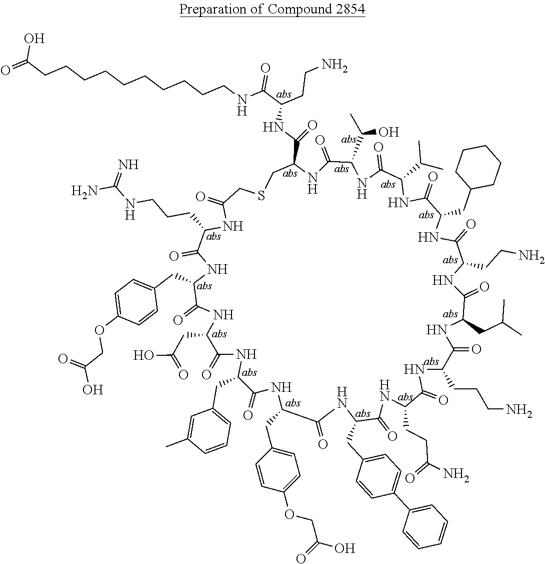 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](Cc3ccc(OCC(=O)O)cc3)NC2=O)c1]

Compound 2854 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 15% B, 15-55% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The yield of the product was 5 mg, and its estimated purity by LCMS analysis was 94.9%. Analysis condition 2: Retention time=1.56 min; ESI-MS(+) m/z [M+2H]²⁺: 1177.

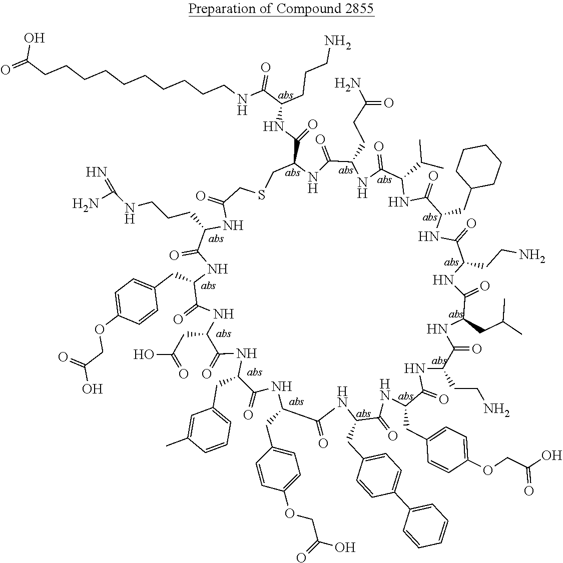 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2855 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 13% B, 13-53% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation.

The yield of the product was 4.9 mg, and its estimated purity by LCMS analysis was 94.9%. Analysis condition 2: Retention time=1.6 min; ESI-MS(+) m/z [M+2H]²⁺: 1237.1.

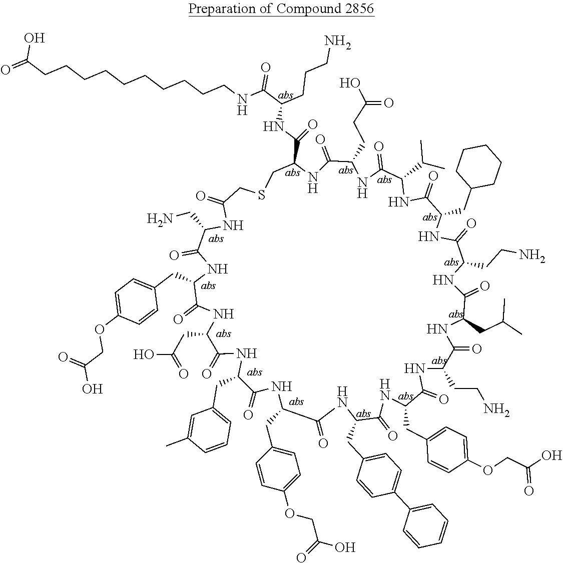 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2856 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 11% B, 11-51% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 5.3 mg, and its estimated purity by LCMS analysis was 95.5%. Analysis condition 2: Retention time=1.59 min; ESI-MS(+) m/z [M+2H]²⁺: 1202.1.

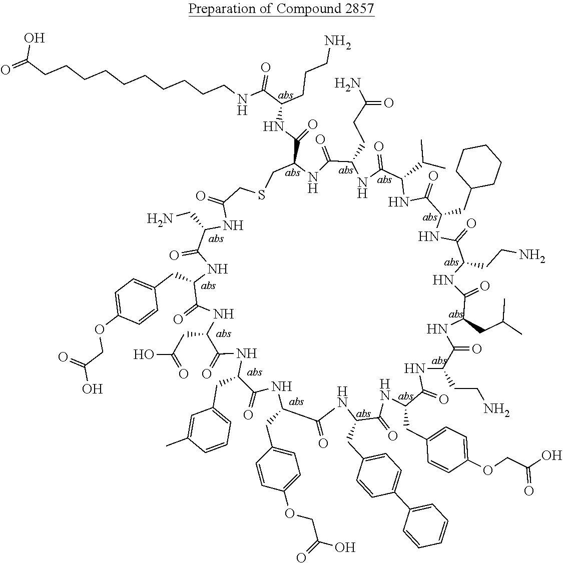 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2857 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 12% B, 12-52% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 1.6 mg, and its estimated purity by LCMS analysis was 100%. Analysis condition 1: Retention time=1.45 min; ESI-MS(+) m/z [M+2H]²⁺: 1201.2.

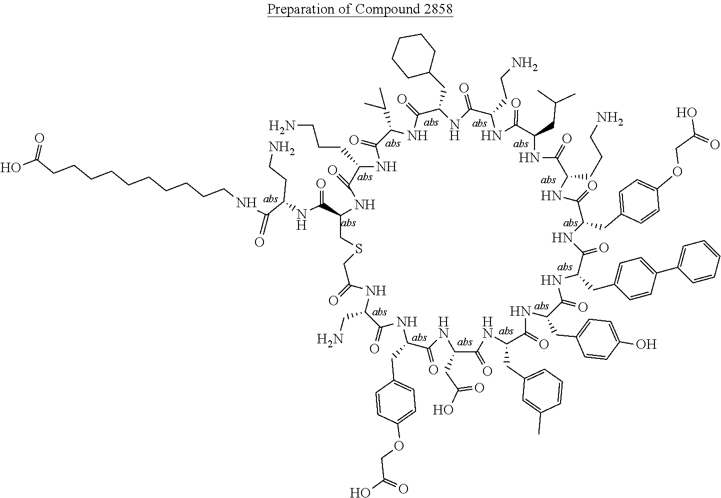 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2858 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 8.3 mg, and its estimated purity by LCMS analysis was 94.8%. Analysis condition 2: Retention time=1.5 min; ESI-MS(+) m/z [M+3H]³⁺: 777.2.

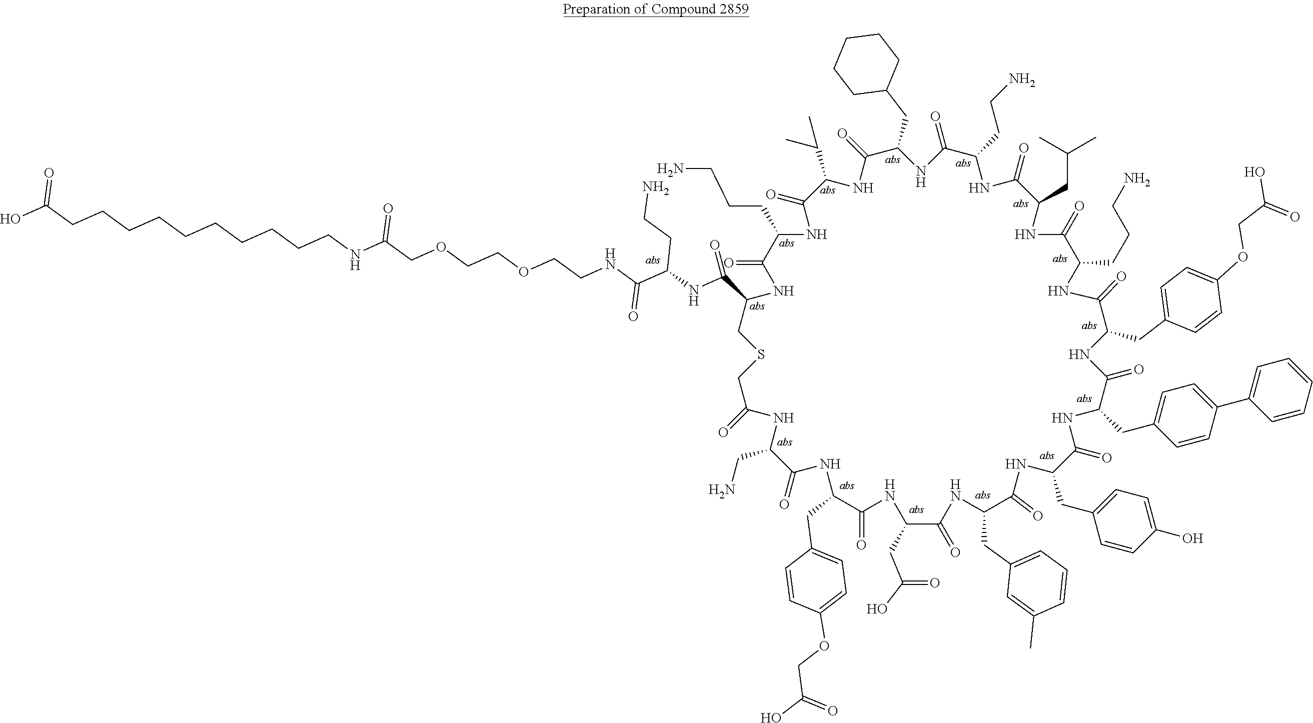 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2859 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 150 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 9.2 mg, and its estimated purity by LCMS analysis was 96.2%. Analysis condition 1: Retention time=1.5 min; ESI-MS(+) m/z [M+3H]³⁺: 826.

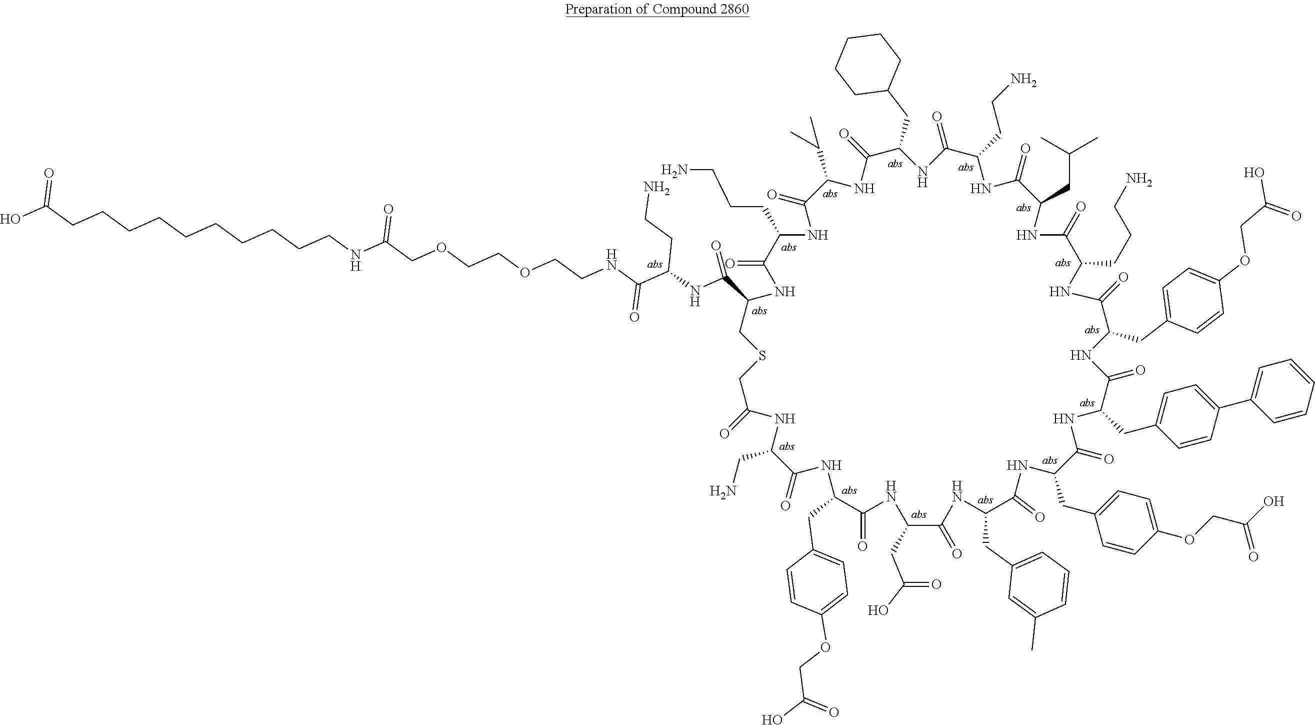 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2860 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0-minute hold at 15% B, 15-55% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 12.7 mg, and its estimated purity by LCMS analysis was 95.2%. Analysis condition 1: Retention time=1.45 min; ESI-MS(+) m/z [M+3H]³⁺: 845.1.

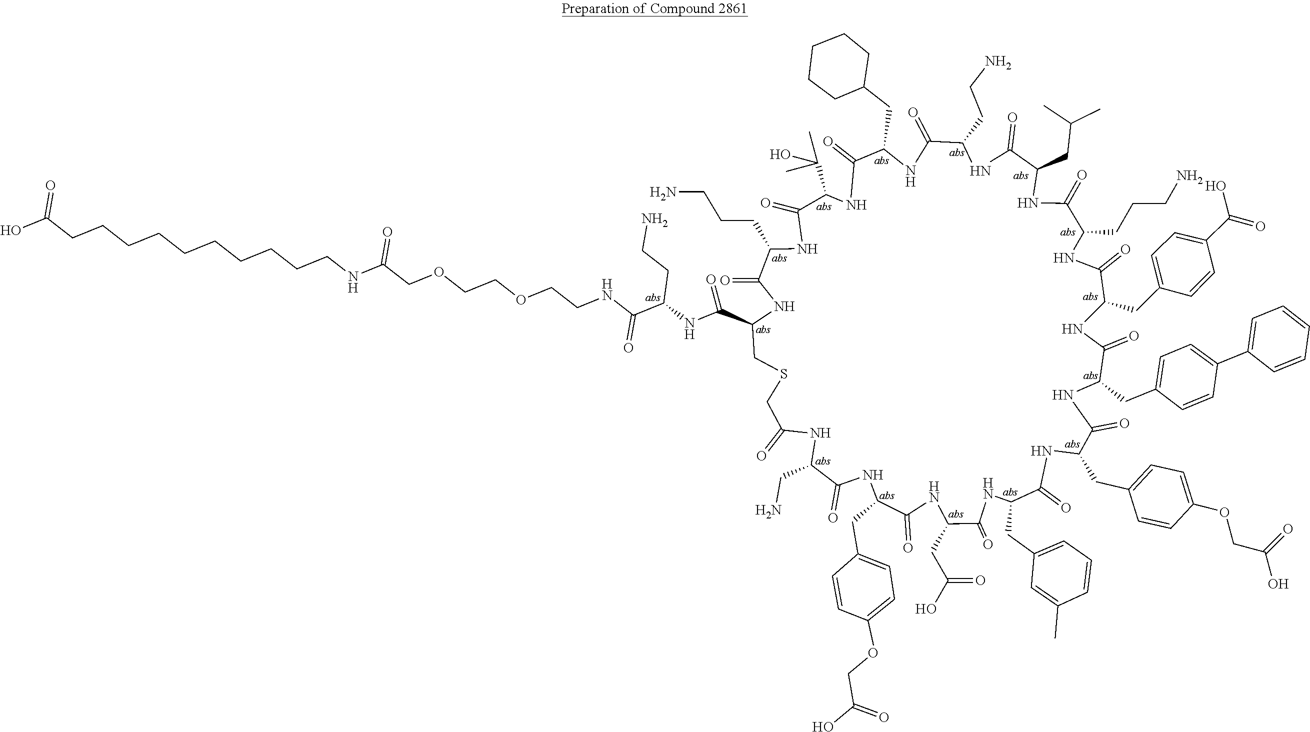 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(OCC(=O)O)cc3)NC2=O)c1]

Compound 2861 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 10% B, 10-50% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 9.8 mg, and its estimated purity by LCMS analysis was 98.1%. Analysis condition B: Retention time=1.56 min; ESI-MS(+) m/z [M+3H]³⁺: 850.2.

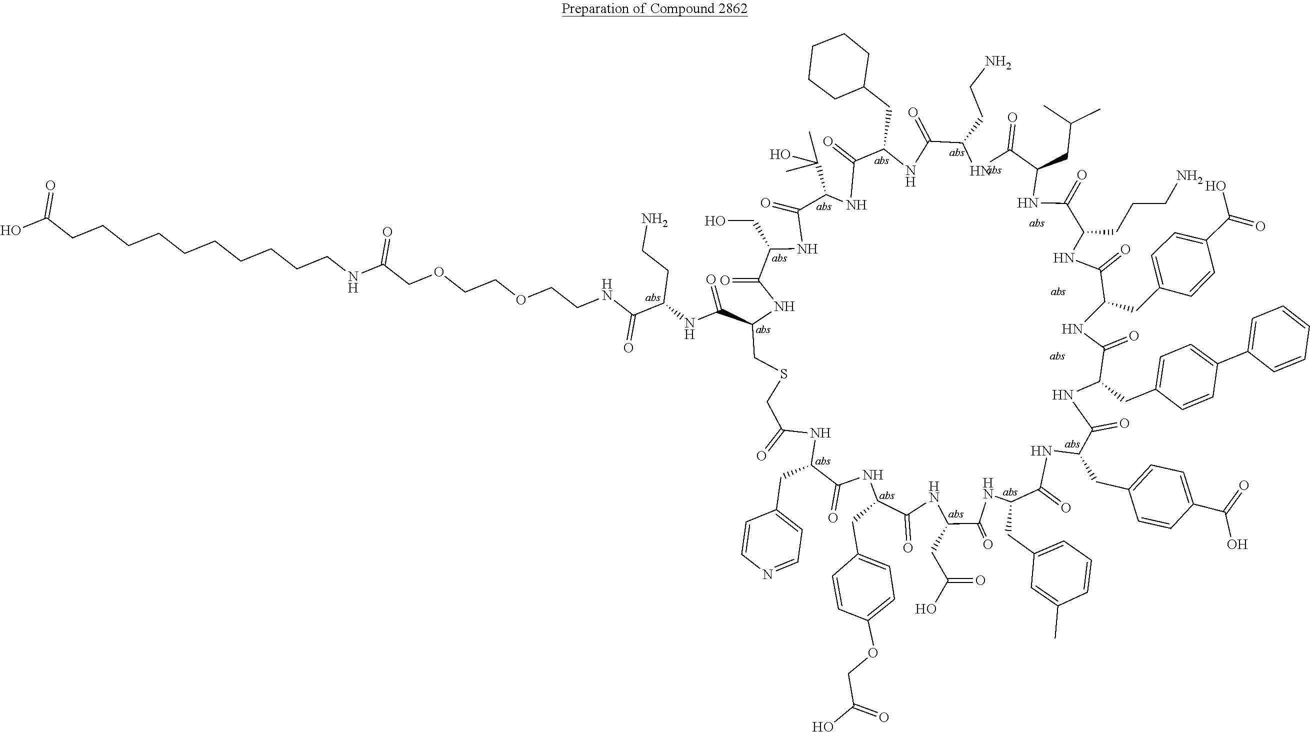 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2862 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 10.5 mg, and its estimated purity by LCMS analysis was 97.7%. Analysis condition B: Retention time=1.67 min; ESI-MS(+) m/z [M+3H]³⁺: 842.2.

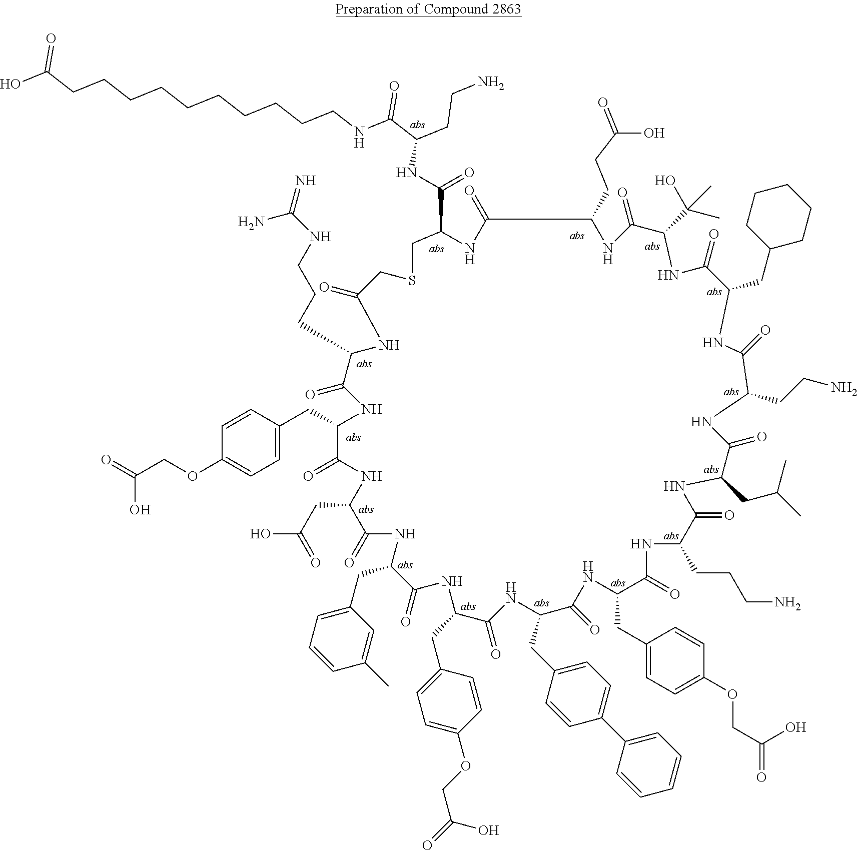 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2863 was prepared, using 2-Chlorotrityl resin pre-loaded with 11-((((9H-fluoren-9-yl) methoxy) carbonyl)amino) undecanoic acid on a −1 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 11% B, 11-51% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 14.8 mg, and its estimated purity by LCMS analysis was 90.9%. Analysis condition 2: Retention time=1.59 min; ESI-MS(+) m/z [M+2H]²⁺: 1244.9.

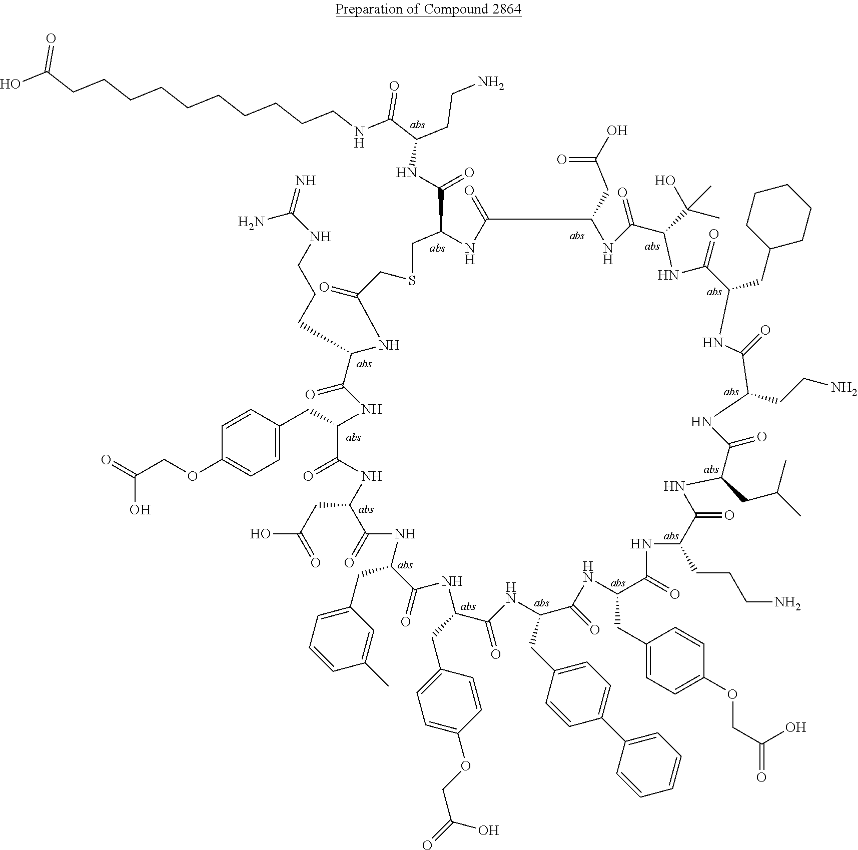 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2864 was prepared, using 2-Chlorotrityl resin pre-loaded with 11-((((9H-fluoren-9-yl) methoxy) carbonyl)amino) undecanoic acid on a −1 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 10% B, 10-50% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 16% B, 16-56% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 1 mg, and its estimated purity by LCMS analysis was 95.2%. Analysis condition 2: Retention time=1.57 min; ESI-MS(+) m/z [M+2H]²⁺: 1238.1.

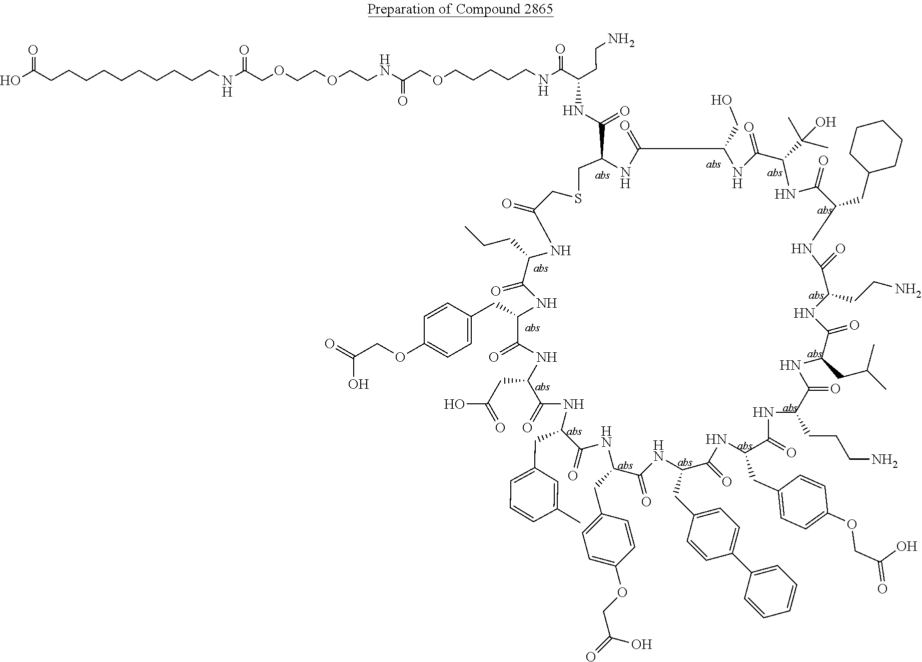 [SMILES: CCC[C@@H]1NC(=O)CSC[C@@H](C(=O)N[C@@H](CCN)C(=O)NCCCCCOCC(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2865 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 33.3 mg, and its estimated purity by LCMS analysis was 98.6%. Analysis condition 2: Retention time=1.88 min; ESI-MS(+) m/z [M+3H]³⁺: 903.1.

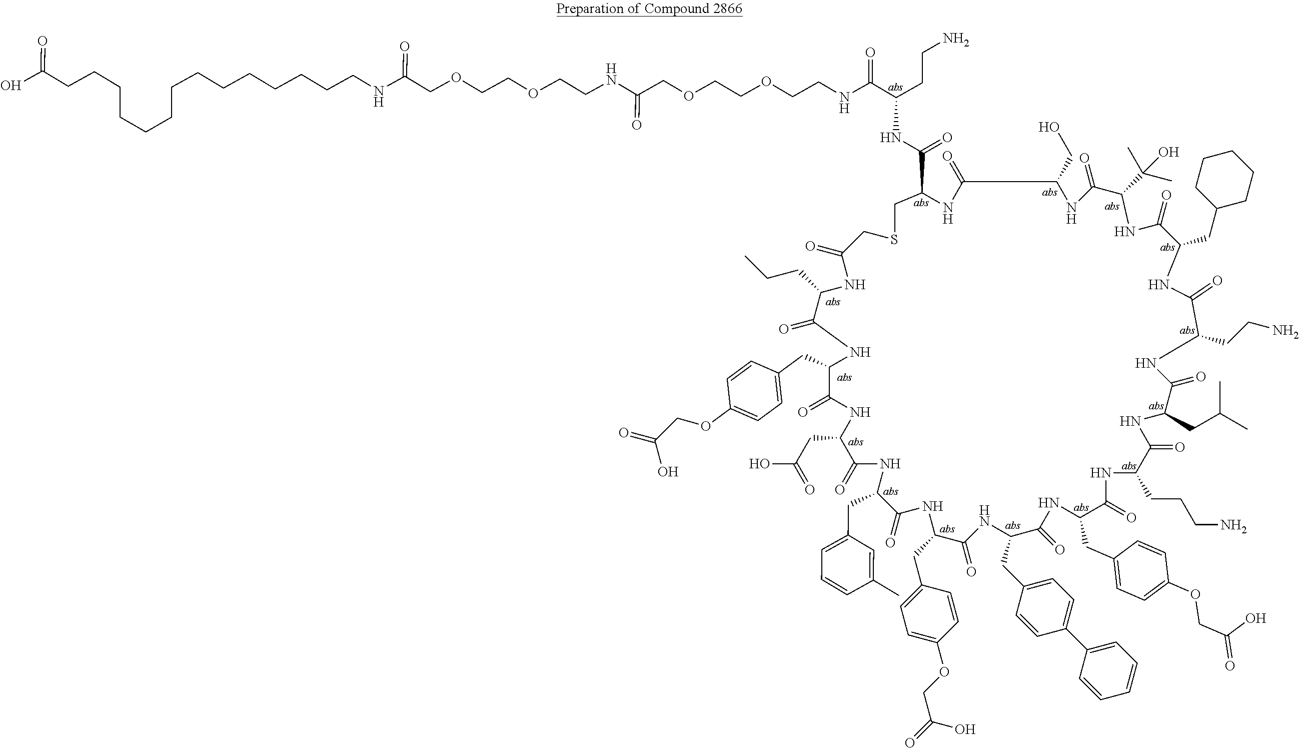 [SMILES: CCC[C@@H]1NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2866 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 29% B, 29-69% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 25.5 mg, and its estimated purity by LCMS analysis was 97.3%. Analysis condition A: Retention time=1.8 min; ESI-MS(+) m/z [M+3H]³⁺: 913.1.

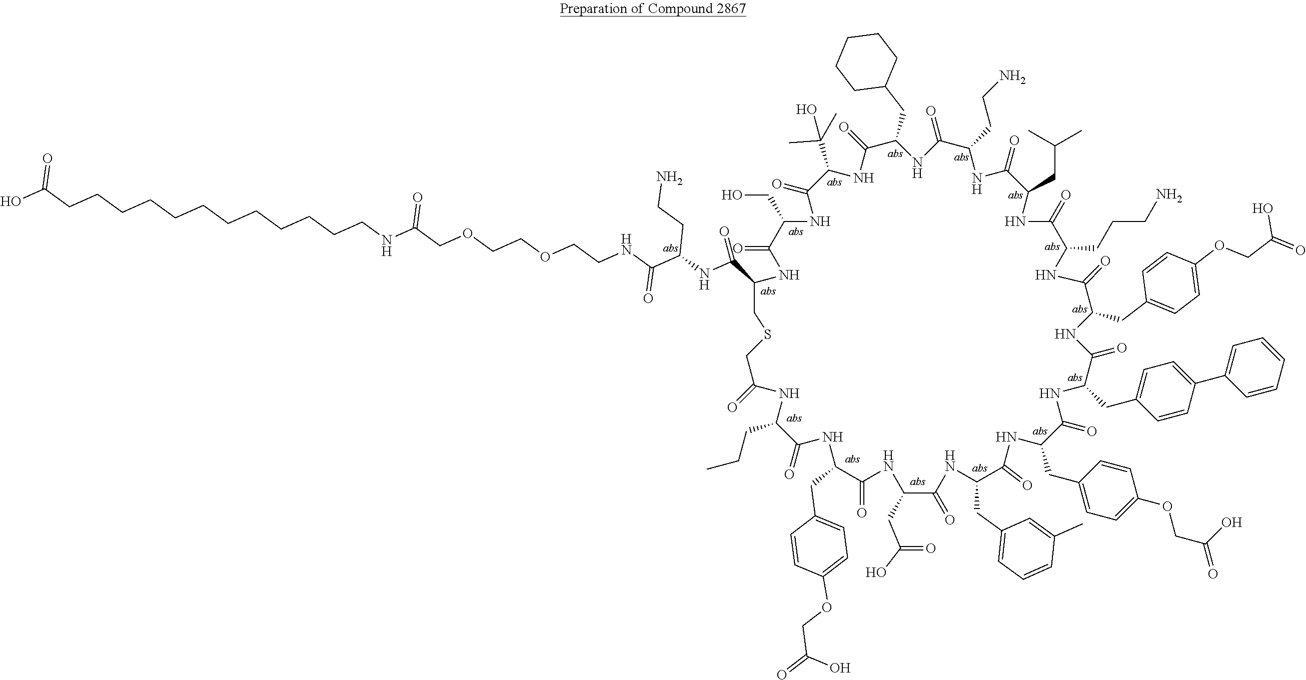 [SMILES: CCC[C@@H]1NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2867 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 28.2 mg, and its estimated purity by LCMS analysis was 100%. Analysis condition A: Retention time=1.59 min; ESI-MS(+) m/z [M+2H]²⁺: 1282.

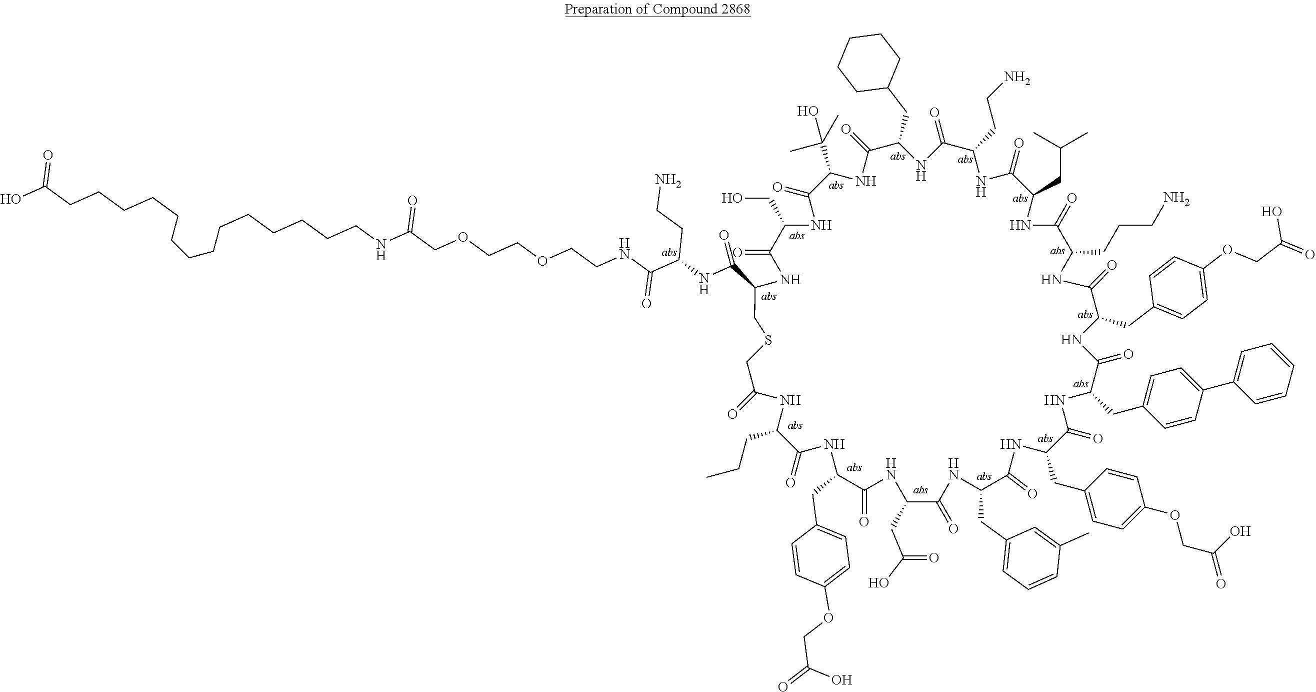 [SMILES: CCC[C@@H]1NC(=O)CSC[C@@H](C(=O)N[C@@H](CCN)C(=O)NCCOCCOCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2868 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 26% B, 26-66% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 11.2 mg, and its estimated purity by LCMS analysis was 99.2%. Analysis condition 2: Retention time=2 min; ESI-MS(+) m/z [M+3H]³⁺: 864.1.

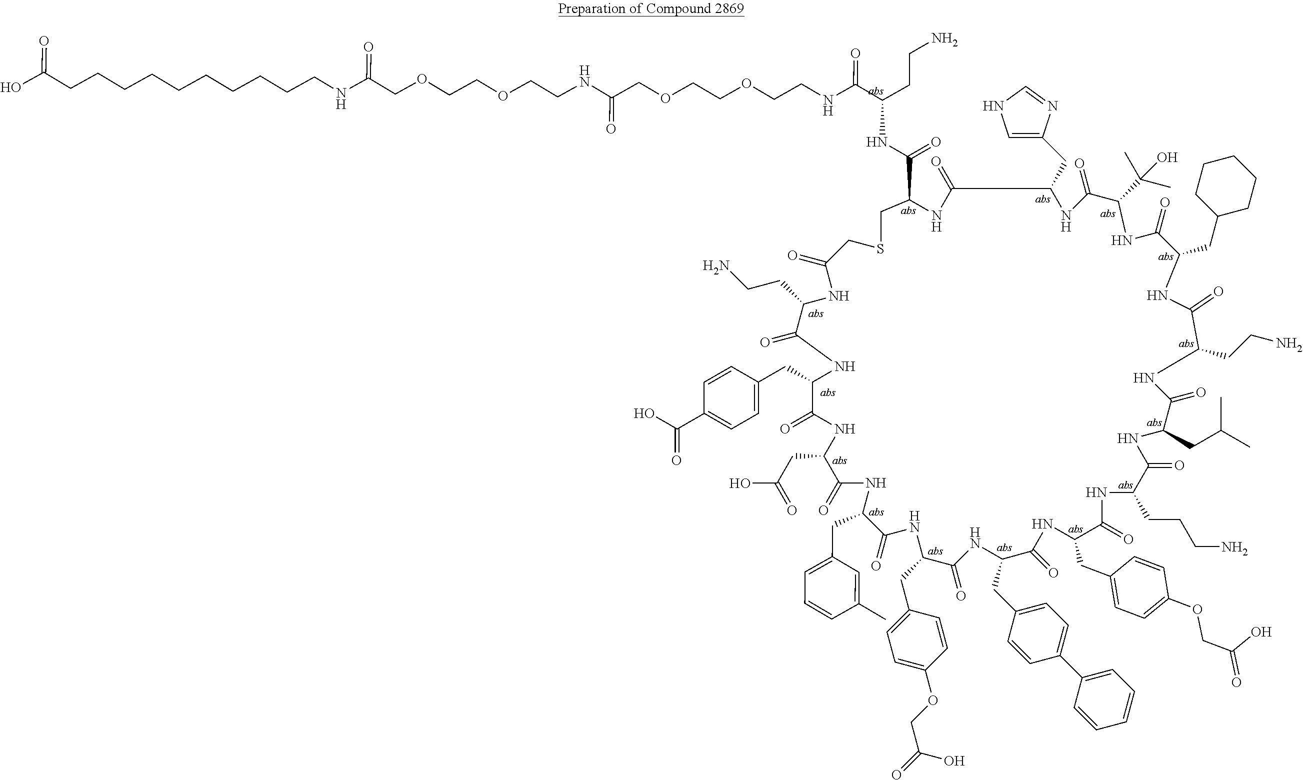 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3c[nH]cn3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2869 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 18 mg, and its estimated purity by LCMS analysis was 99.1%. Analysis condition A: Retention time=1.63 min; ESI-MS(+) m/z [M+2H]²⁺: 1365.1.

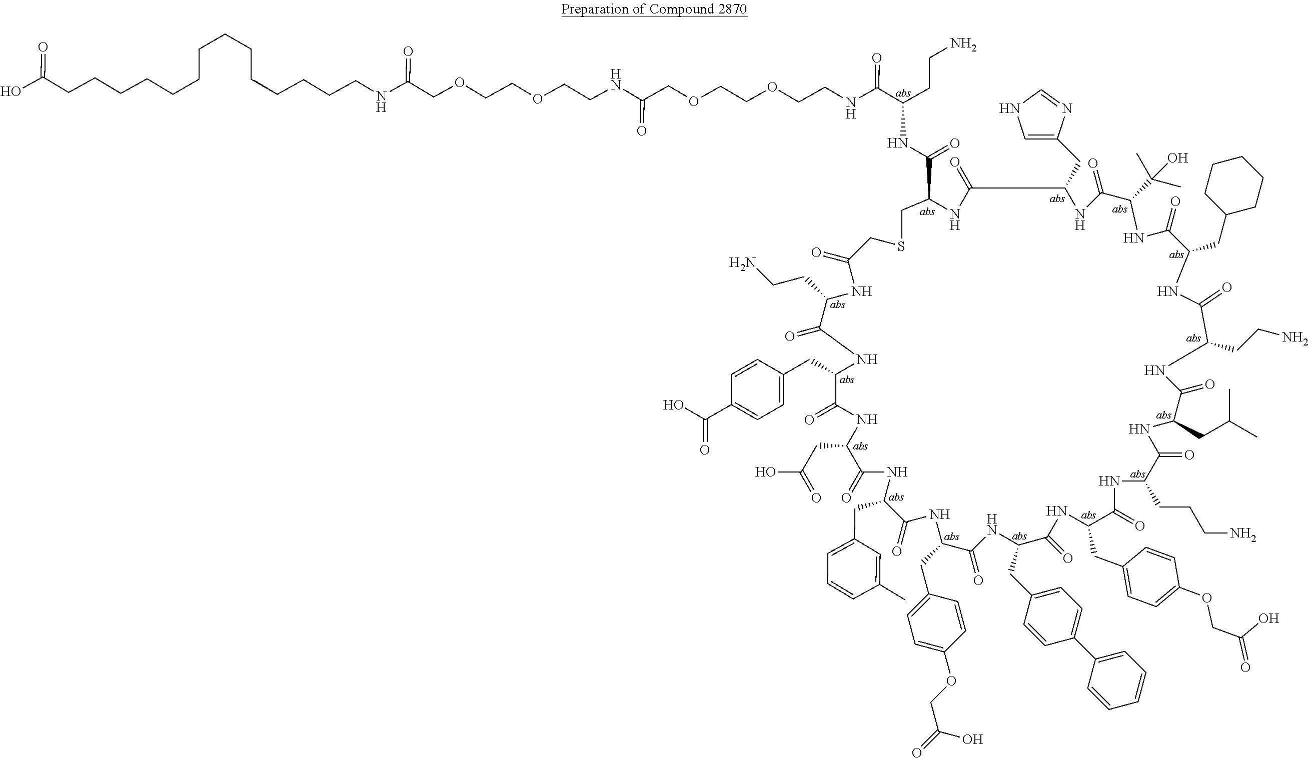 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCC(C)CCCCCCCCCCC(=O)O)NC(=O)[C@H](Cc3c[nH]cn3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2870 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 18% B, 18-58% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 17.9 mg, and its estimated purity by LCMS analysis was 92.8%. Analysis condition 1: Retention time=1.77 min; ESI-MS(+) m/z [M+3H]³⁺: 920.2.

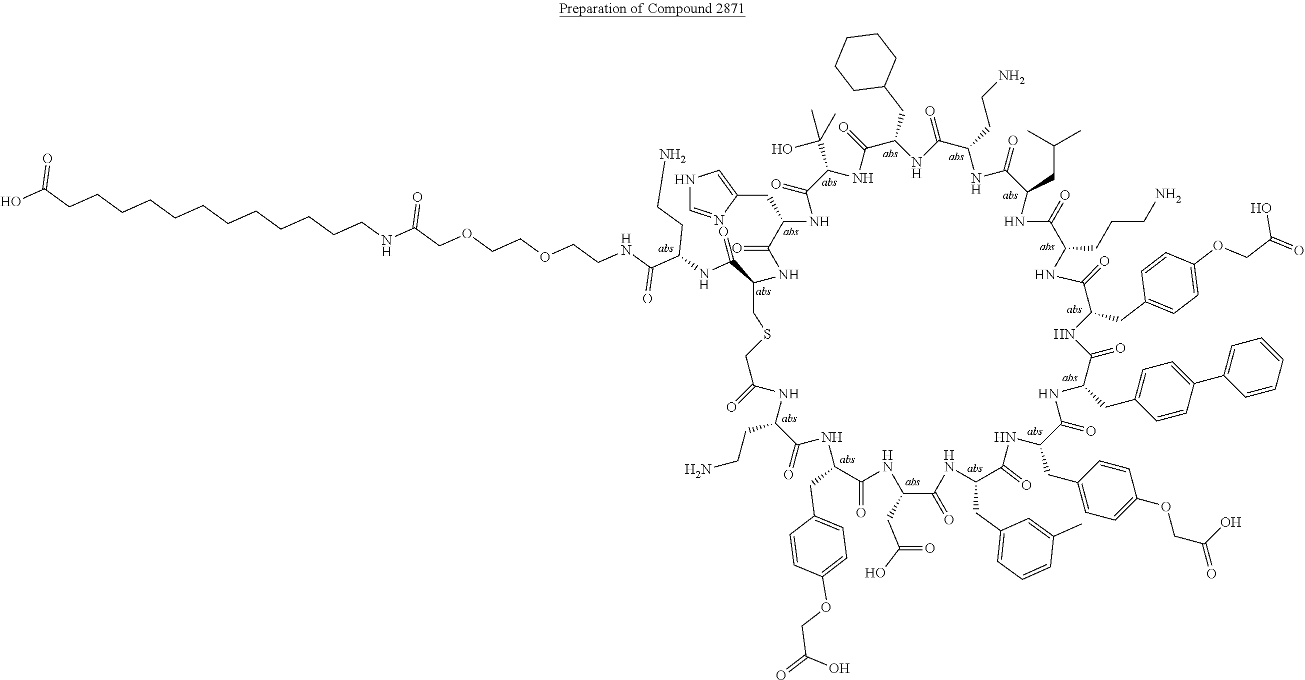 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCCCC(=O)O)NC(=O)[C@H](Cc3c[nH]cn3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2871 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 15 mg, and its estimated purity by LCMS analysis was 92.4%. Analysis condition 2: Retention time=1.62 min; ESI-MS(+) m/z [M+3H]³⁺: 872.

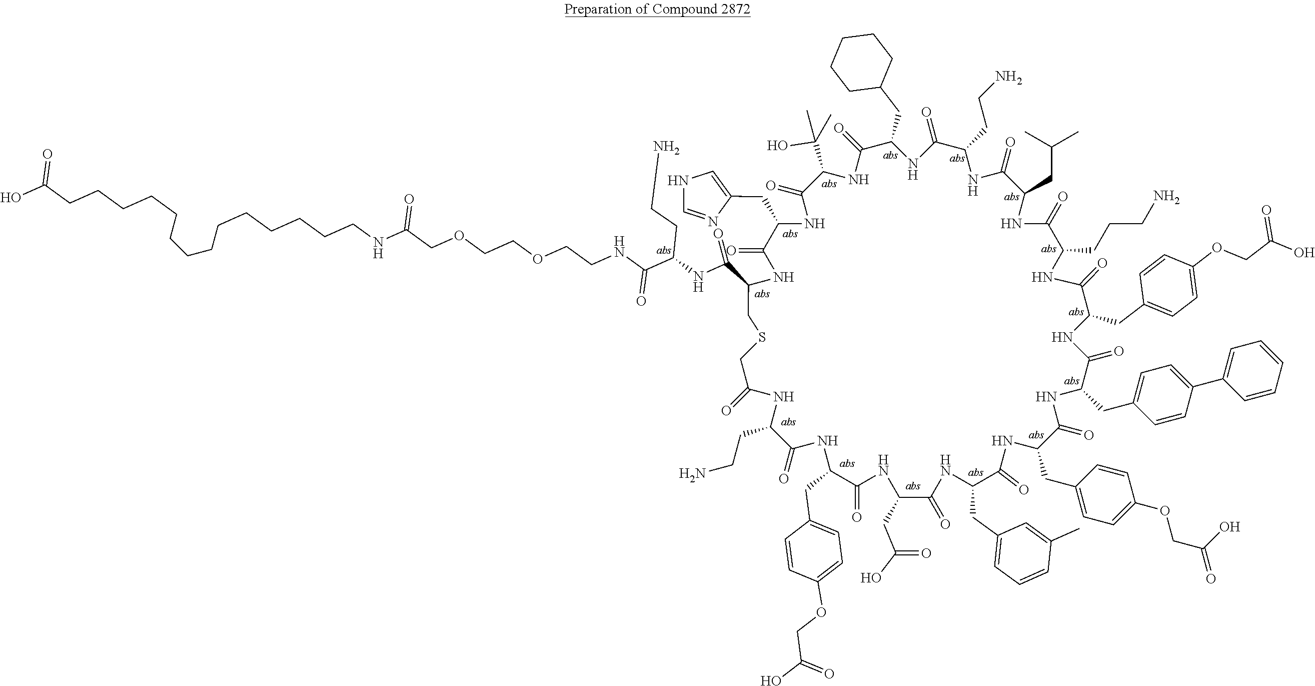 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCCCCCC(=O)O)NC(=O)[C@H](Cc3c[nH]cn3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2872 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 14.4 mg, and its estimated purity by LCMS analysis was 92.3%. Analysis condition 1: Retention time=1.63 min; ESI-MS(+) m/z [M+3H]³⁺: 881.2.

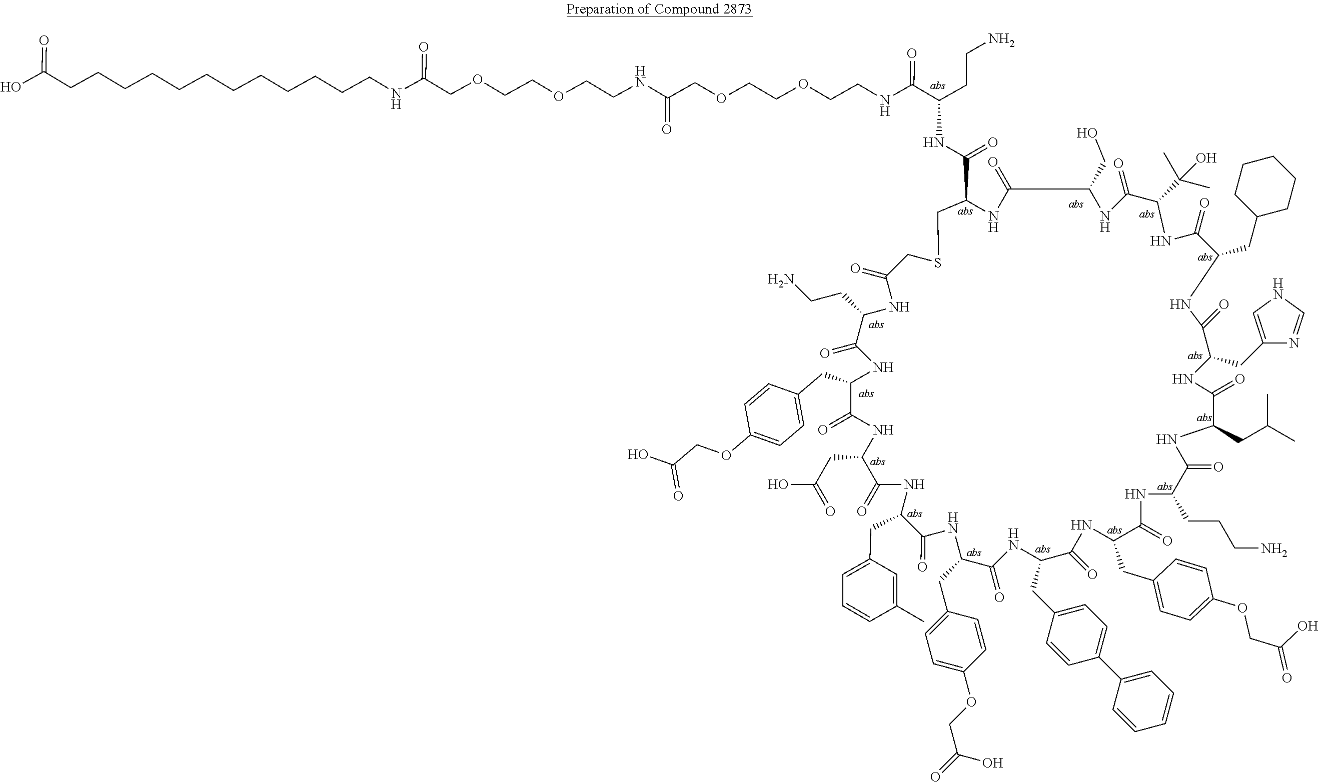 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2873 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 35.8 mg, and its estimated purity by LCMS analysis was 92.7%. Analysis condition 1: Retention time=1.55 min; ESI-MS(+) m/z [M+2H]²⁺: 1373.5.

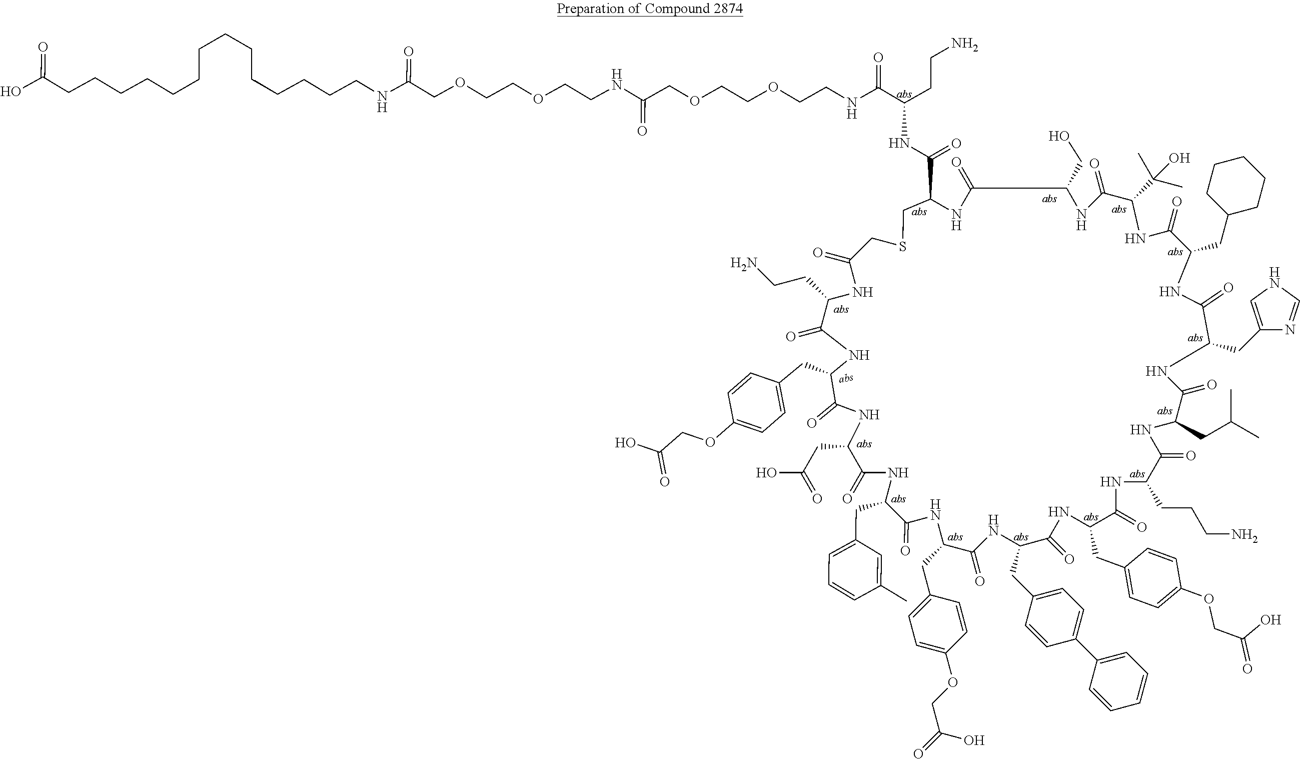 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCC(C)CCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2874 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 17% B, 17-57% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 41.9 mg, and its estimated purity by LCMS analysis was 97.4%. Analysis condition B: Retention time=1.8 min; ESI-MS(+) m/z [M+2H]²⁺: 1388.1.

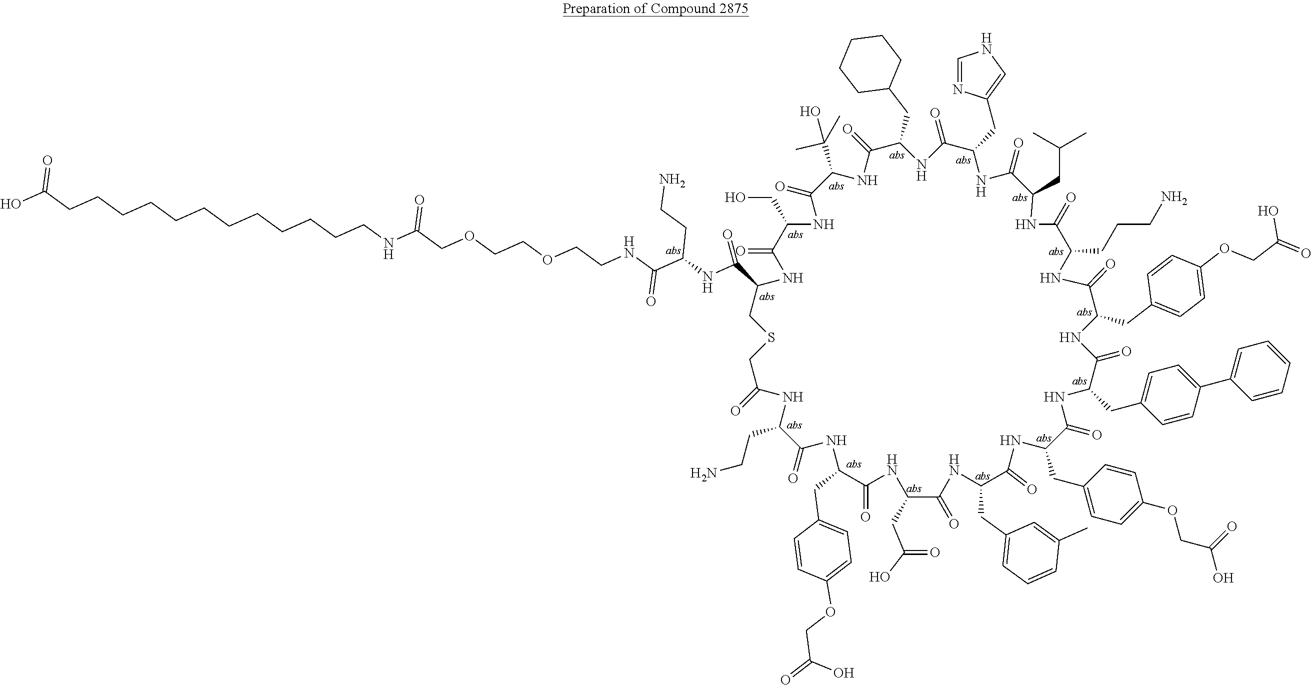 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2875 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 16% B, 16-56% B over 23 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 32.9 mg, and its estimated purity by LCMS analysis was 90.2%. Analysis condition 1: Retention time=1.75 min; ESI-MS(+) m/z [M+2H]²⁺: 1301.

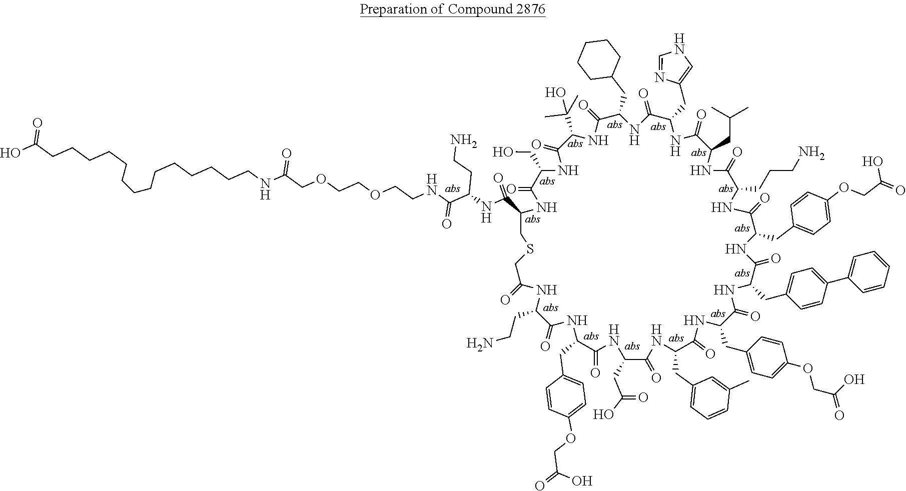 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2876 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 24% B, 24-64% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 27 mg, and its estimated purity by LCMS analysis was 98.5%. Analysis condition A: Retention time=1.84 min; ESI-MS(+) m/z [M+2H]²⁺: 1315.2.

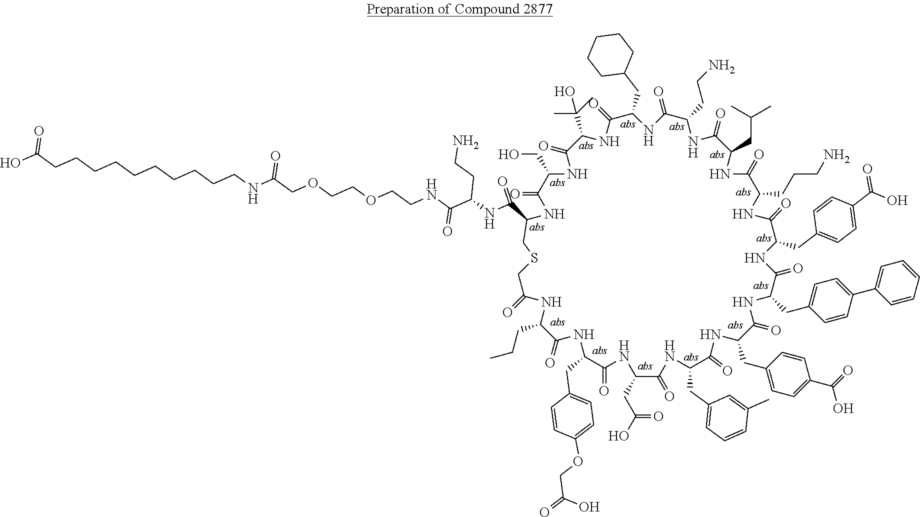 [SMILES: CCC[C@@H]1NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2877 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 19.4 mg, and its estimated purity by LCMS analysis was 98.9%. Analysis condition 1: Retention time=1.55 min; ESI-MS(+) m/z [M+2H]²⁺: 1238.1.

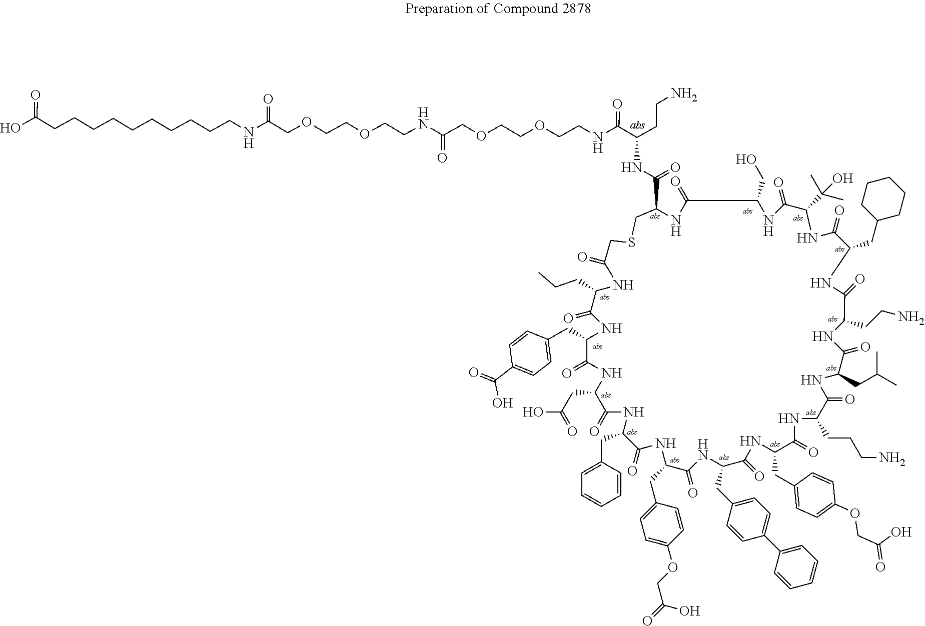 [SMILES: CCC[C@@H]1NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2ccccc2)NC(=O)[C@H](CC(=O)O)NC(=O)[C@H](Cc2ccc(C(=O)O)cc2)NC1=O]

Compound 2878 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 22% B, 22-62% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 40 mg, and its estimated purity by LCMS analysis was 100%. Analysis condition A: Retention time=1.53 min; ESI-MS(+) m/z [M+3H]³⁺: 894.3.

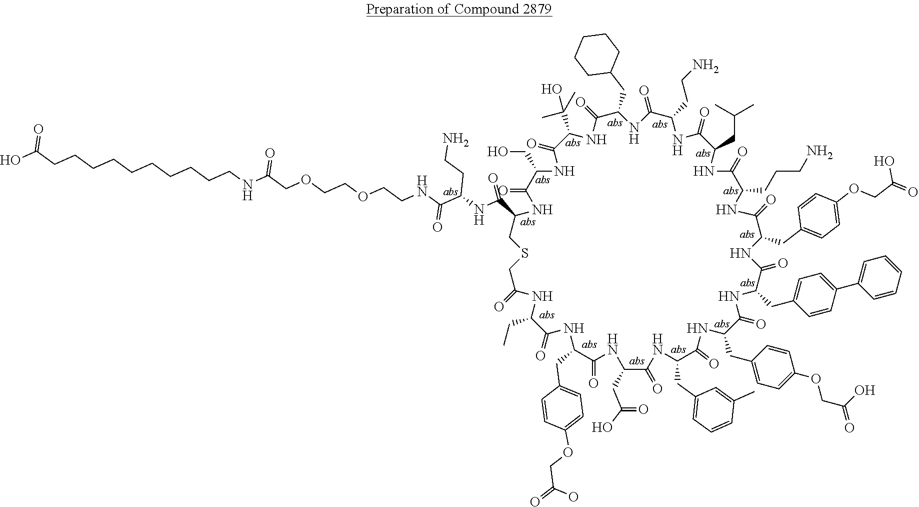 [SMILES: CC[C@@H]1NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2879 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 26.7 mg, and its estimated purity by LCMS analysis was 97.3%. Analysis condition 2: Retention time=1.72 min; ESI-MS(+) m/z [M+2H]²⁺: 1261.2.

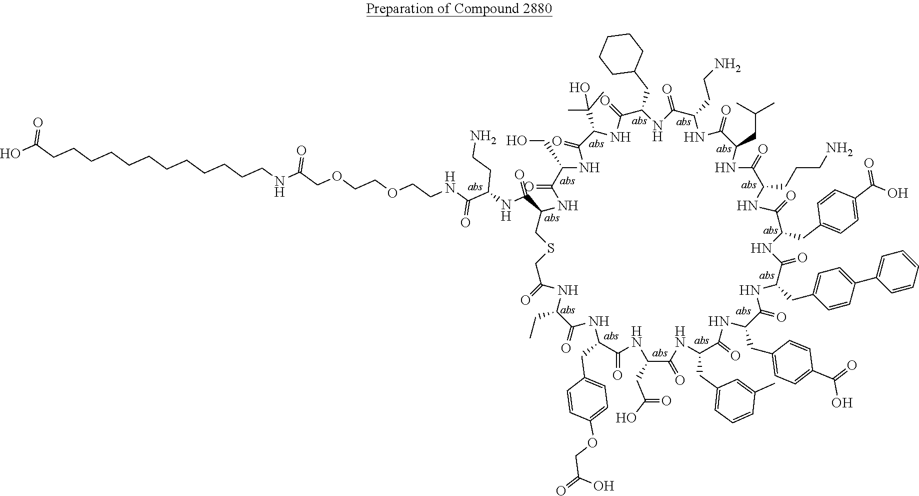 [SMILES: CC[C@@H]1NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2880 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-55% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 16.6 mg, and its estimated purity by LCMS analysis was 99%. Analysis condition 2: Retention time=1.71 min; ESI-MS(+) m/z [M+2H]²⁺: 1231.2.

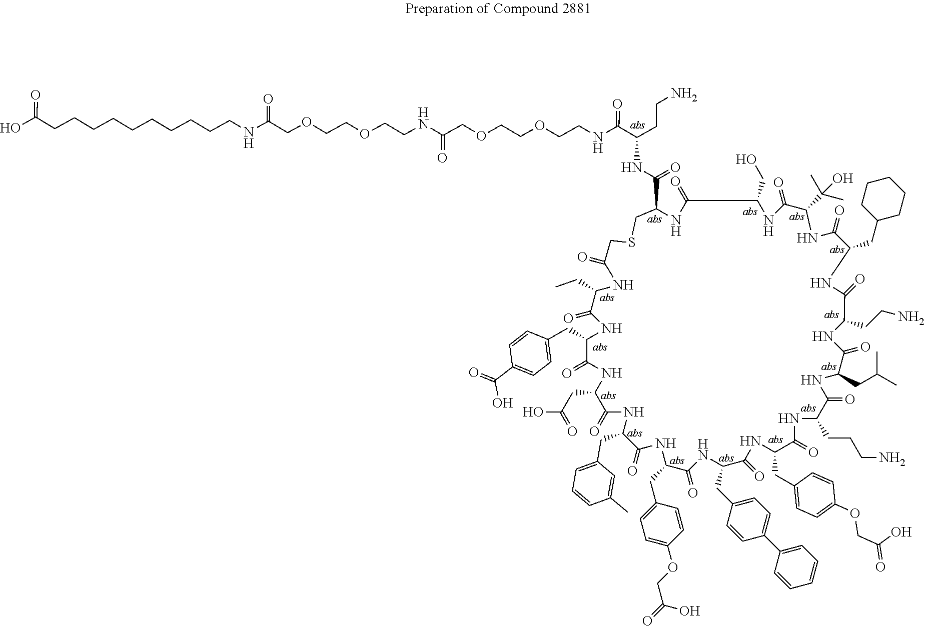 [SMILES: CC[C@@H]1NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Compound 2881 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 21.9 mg, and its estimated purity by LCMS analysis was 100%. Analysis condition A: Retention time=1.46 min; ESI-MS(+) m/z [M+2H]²⁺: 1334.1.

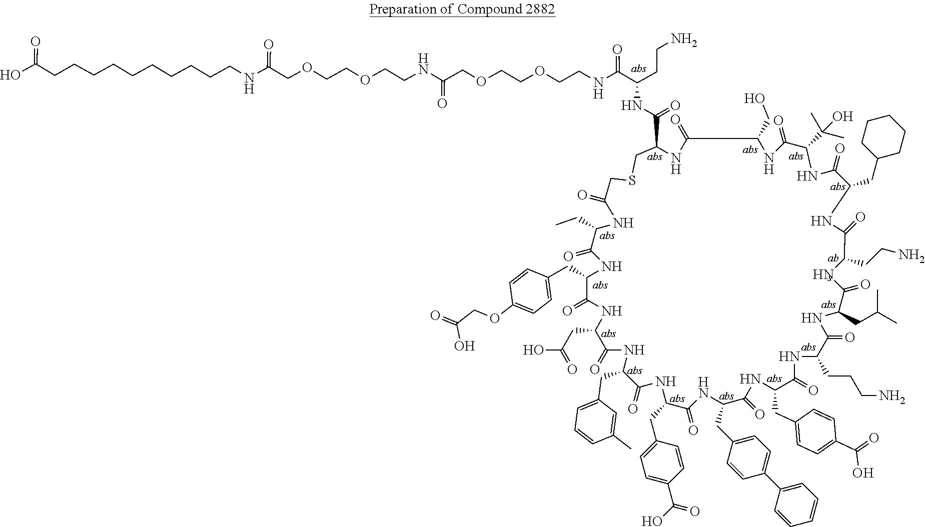 [SMILES: CC[C@@H]1NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2882 was prepared, using 2-Chlorotrityl resin pre-loaded with 11-((((9H-fluoren-9-yl) methoxy) carbonyl)amino) undecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 37.1 mg, and its estimated purity by LCMS analysis was 97.3%. Analysis condition 2: Retention time=1.71 min; ESI-MS(+) m/z [M+2H]²⁺: 1303.8.

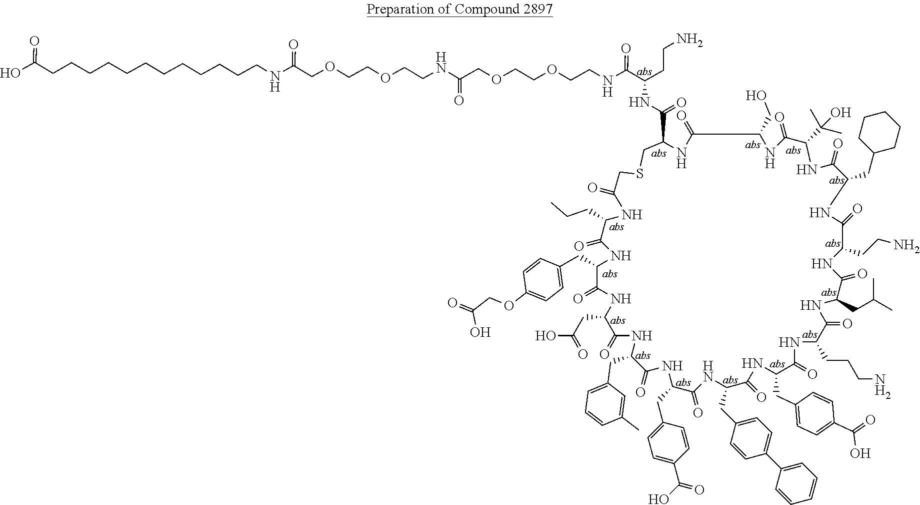 [SMILES: CCC[C@@H]1NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2883 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 24% B, 24-64% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 18 mg, and its estimated purity by LCMS analysis was 98.4%. Analysis condition 2: Retention time=1.8 min; ESI-MS(+) m/z [M+3H]³⁺: 883.3.

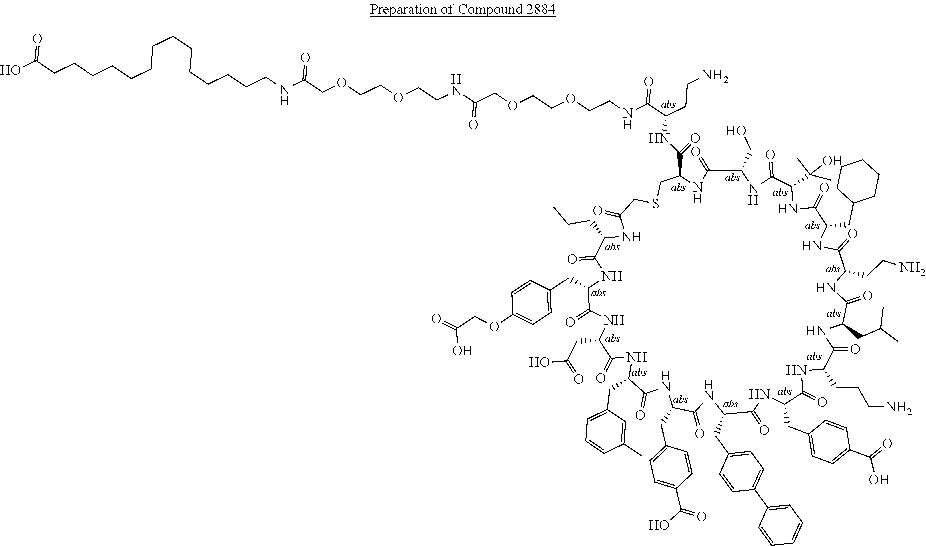 [SMILES: CCC[C@@H]1NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2884 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 28% B, 28-68% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16 mg, and its estimated purity by LCMS analysis was 96.3%. Analysis condition A: Retention time=1.64 min; ESI-MS(+) m/z [M+3H]³⁺: 893.1.

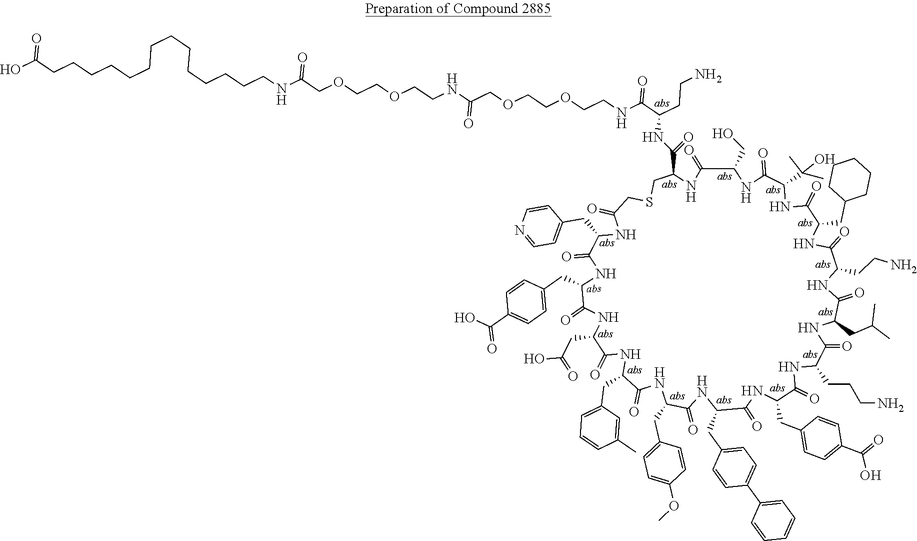 [SMILES: COc1ccc(C[C@@H]2NC(=O)[C@H](Cc3cccc(C)c3)NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC2=O)cc1]

Compound 2885 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 28% B, 28-68% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.3 mg, and its estimated purity by LCMS analysis was 100%. Analysis condition 2: Retention time=1.85 min; ESI-MS(+) m/z [M+2H]²⁺: 1341.

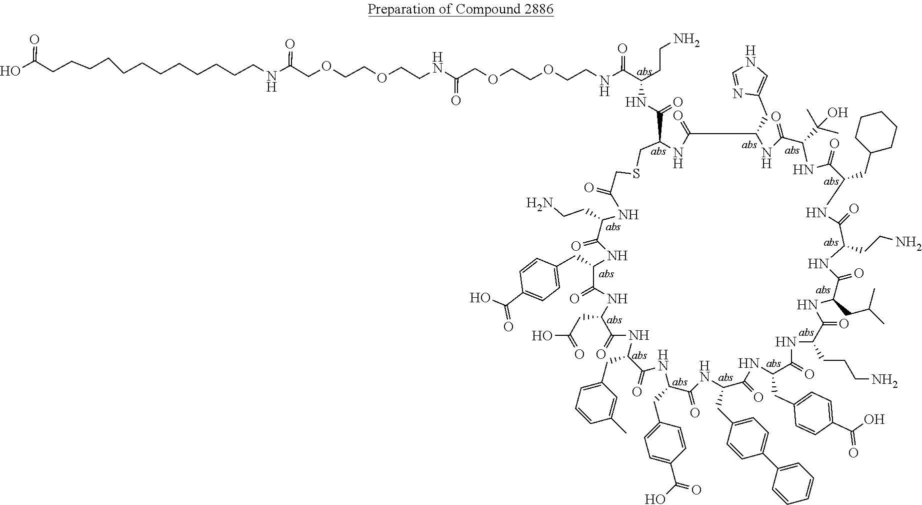 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](Cc3c[nH]cn3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2886 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 19% B, 19-59% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 21.5 mg, and its estimated purity by LCMS analysis was 95.9%. Analysis condition A: Retention time=1.64 min; ESI-MS(+) m/z [M+3H]³⁺: 890.3.

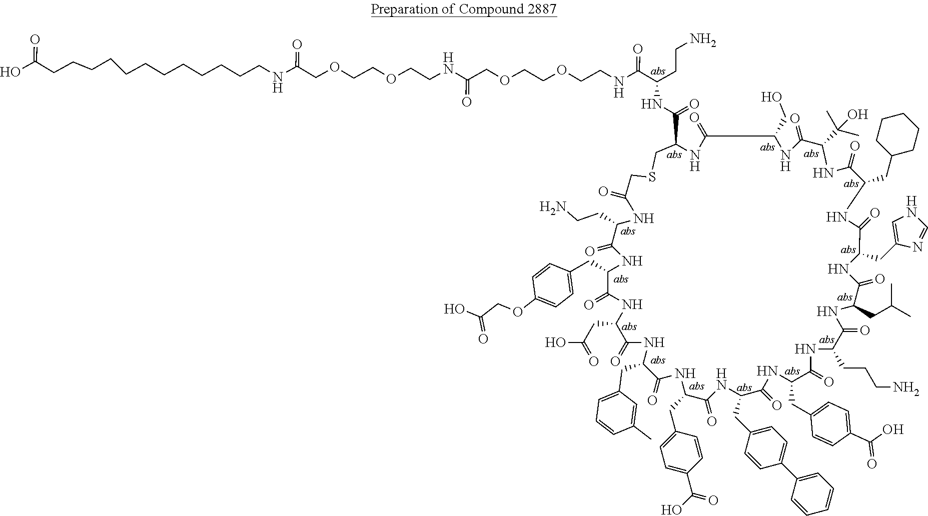 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2887 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 16.3 mg, and its estimated purity by LCMS analysis was 96.5%. Analysis condition A: Retention time=1.71 min; ESI-MS(+) m/z [M+2H]²⁺: 895.9.

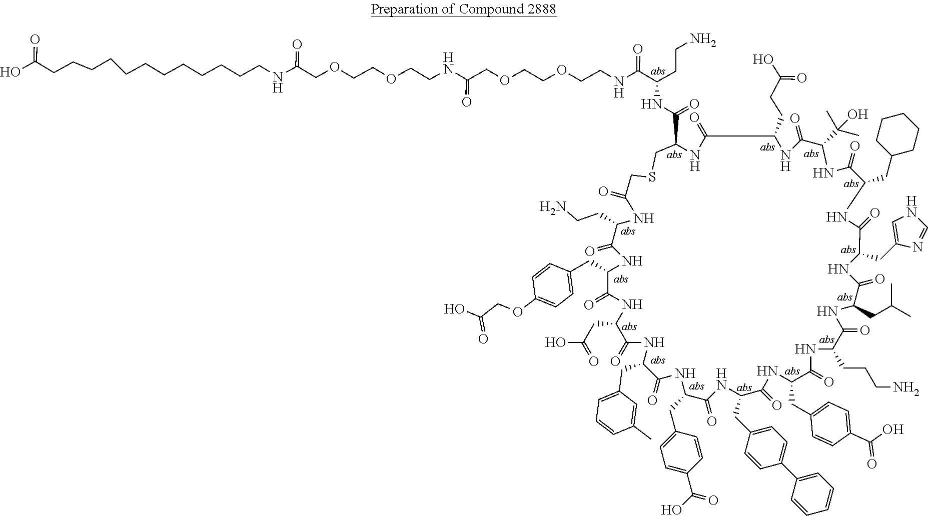 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CCC(=O)O)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2888 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 20% B, 20-60% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 20.9 mg, and its estimated purity by LCMS analysis was 95.6%. Analysis condition B: Retention time=1.61 min; ESI-MS(+) m/z [M+3H]³⁺: 910.2.

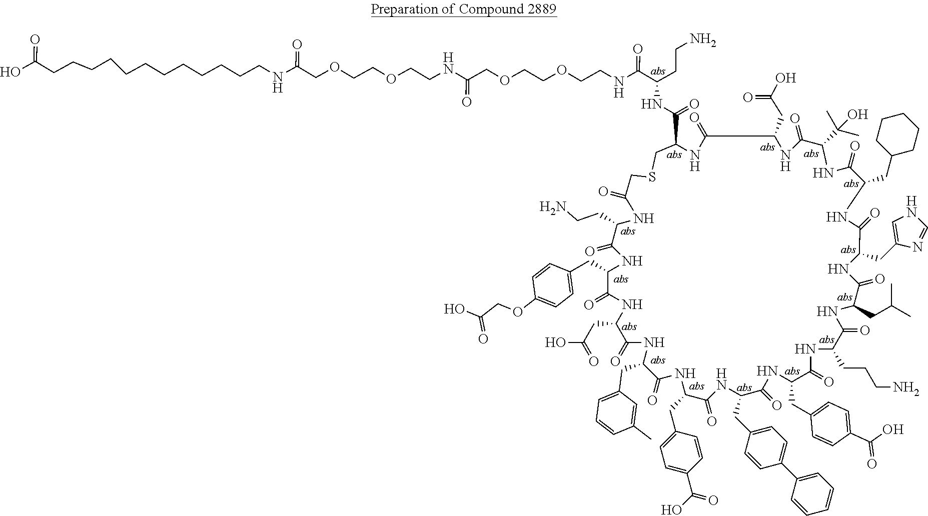 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CC(=O)O)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2889 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 12% B, 12-52% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 32.6 mg, and its estimated purity by LCMS analysis was 100%. Analysis condition A: Retention time=1.59 min; ESI-MS(+) m/z [M+2H]²⁺: 1358.

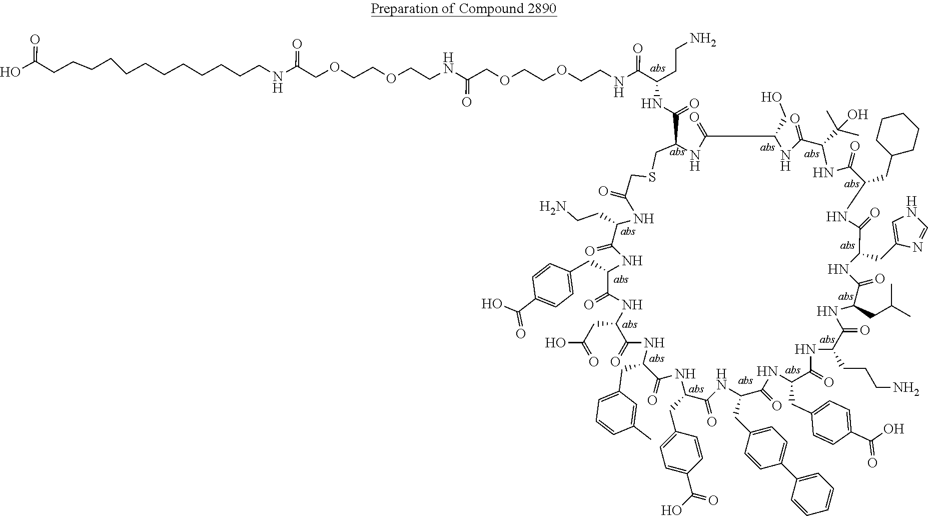 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2890 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 15.5 mg, and its estimated purity by LCMS analysis was 97.6%. Analysis condition B: Retention time=1.61 min; ESI-MS(+) m/z [M+2H]²⁺: 1329.1.

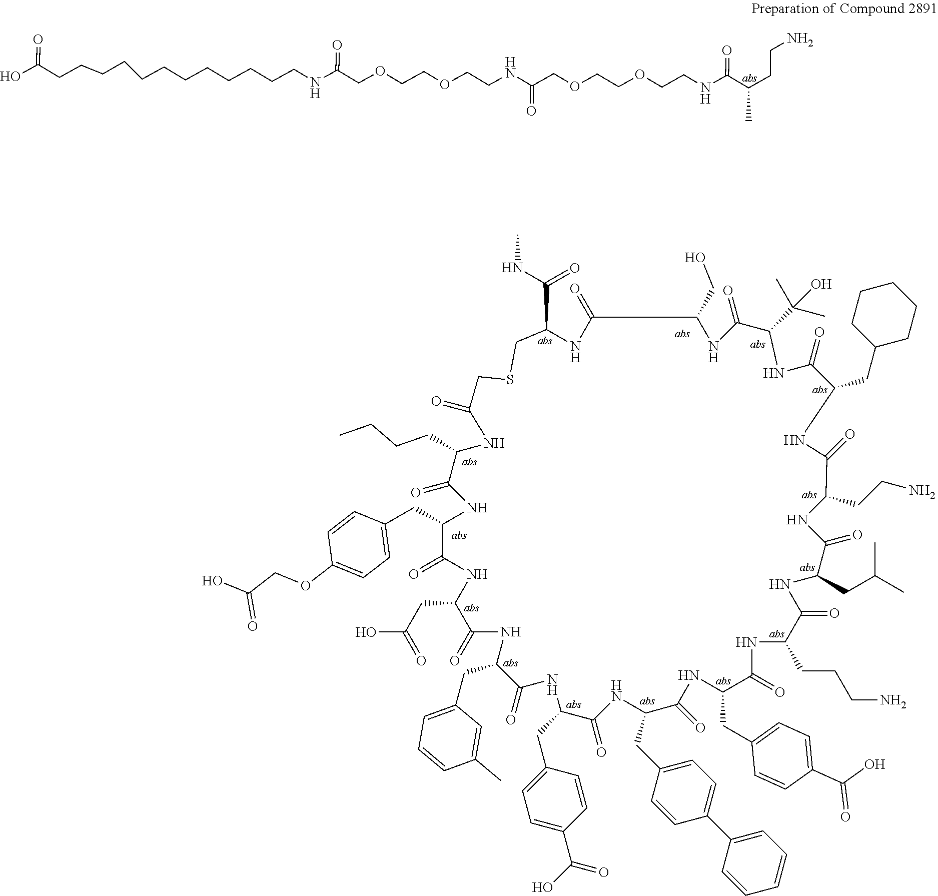

Compound 2891 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-60% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 26% B, 26-66% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 8 mg, and its estimated purity by LCMS analysis was 98%. Analysis condition B: Retention time=1.93 min; ESI-MS(+) m/z [M+3H]³⁺: 888.3.

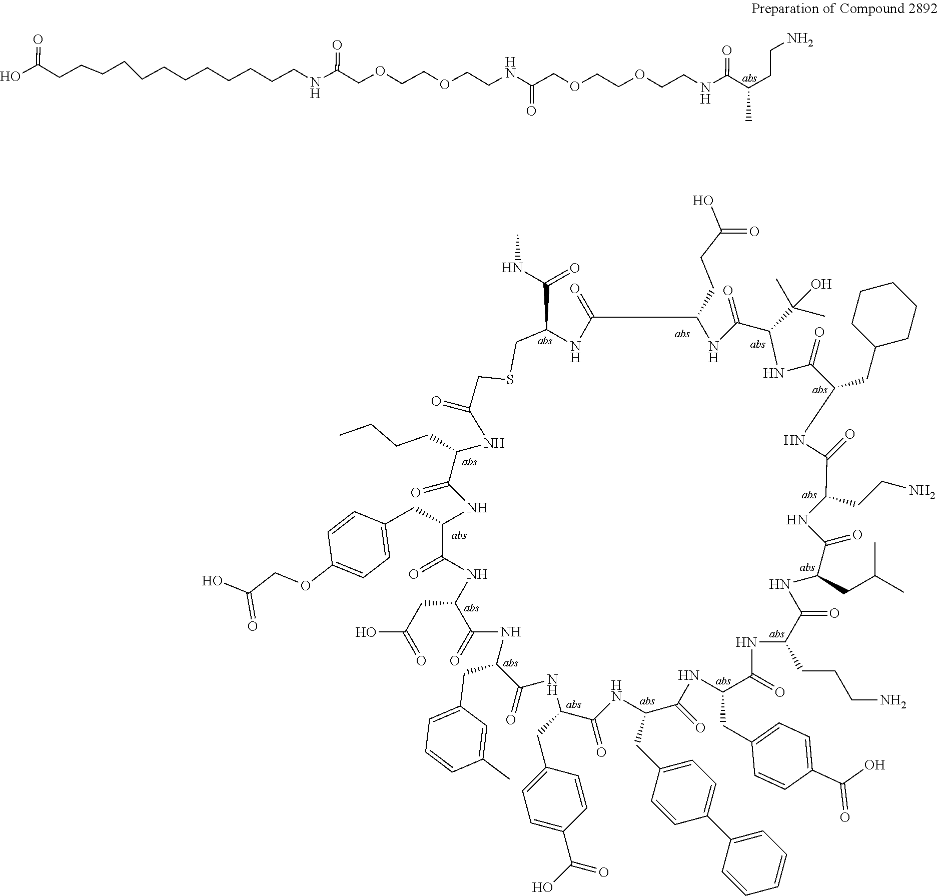

Compound 2892 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 46.8 mg, and its estimated purity by LCMS analysis was 100%. Analysis condition A: Retention time=1.48 min; ESI-MS(+) m/z [M+2H]²⁺: 1353.

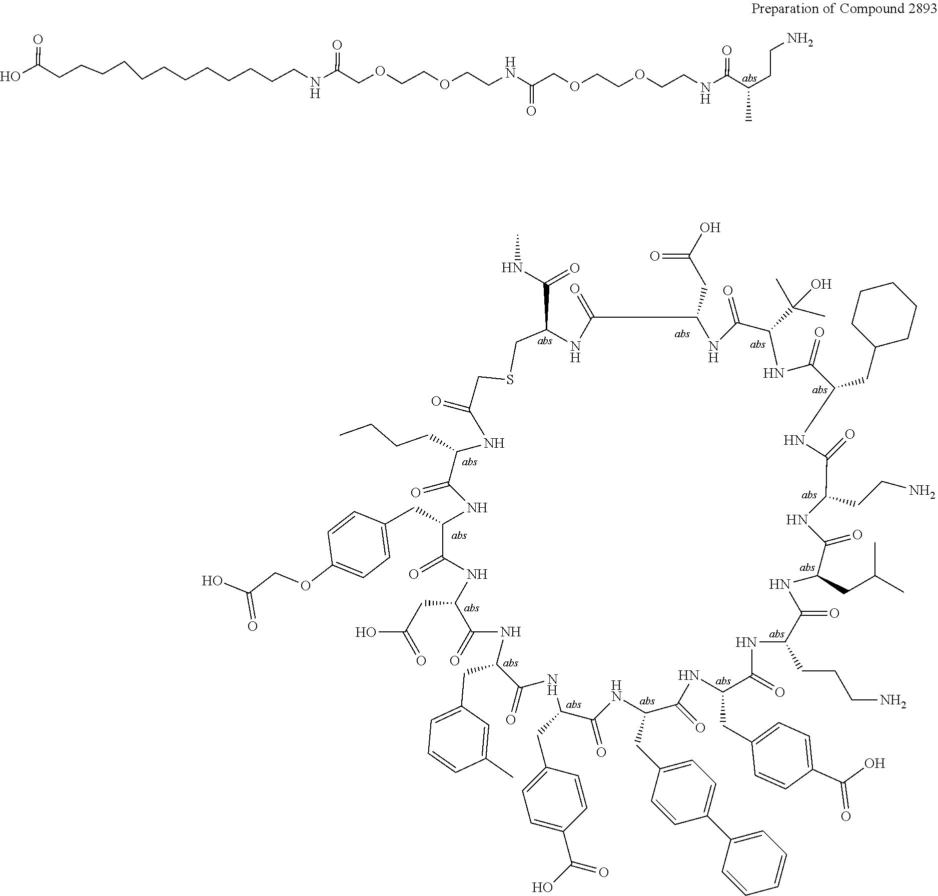

Compound 2893 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 18.1 mg, and its estimated purity by LCMS analysis was 94.4%. Analysis condition B: Retention time=1.87 min; ESI-MS(+) m/z [M+3H]³⁺: 897.2.

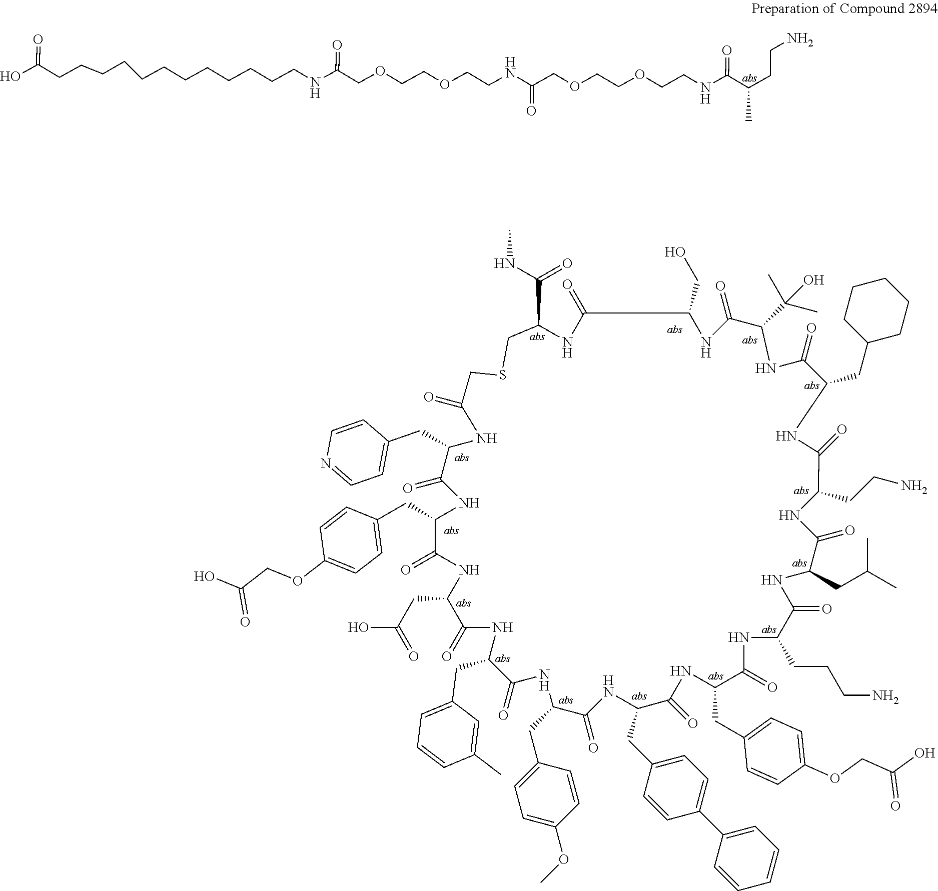

Compound 2894 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 26.1 mg, and its estimated purity by LCMS analysis was 97.1%. Analysis condition A: Retention time=1.64 min; ESI-MS(+) m/z [M+2H]²⁺: 1357.2.

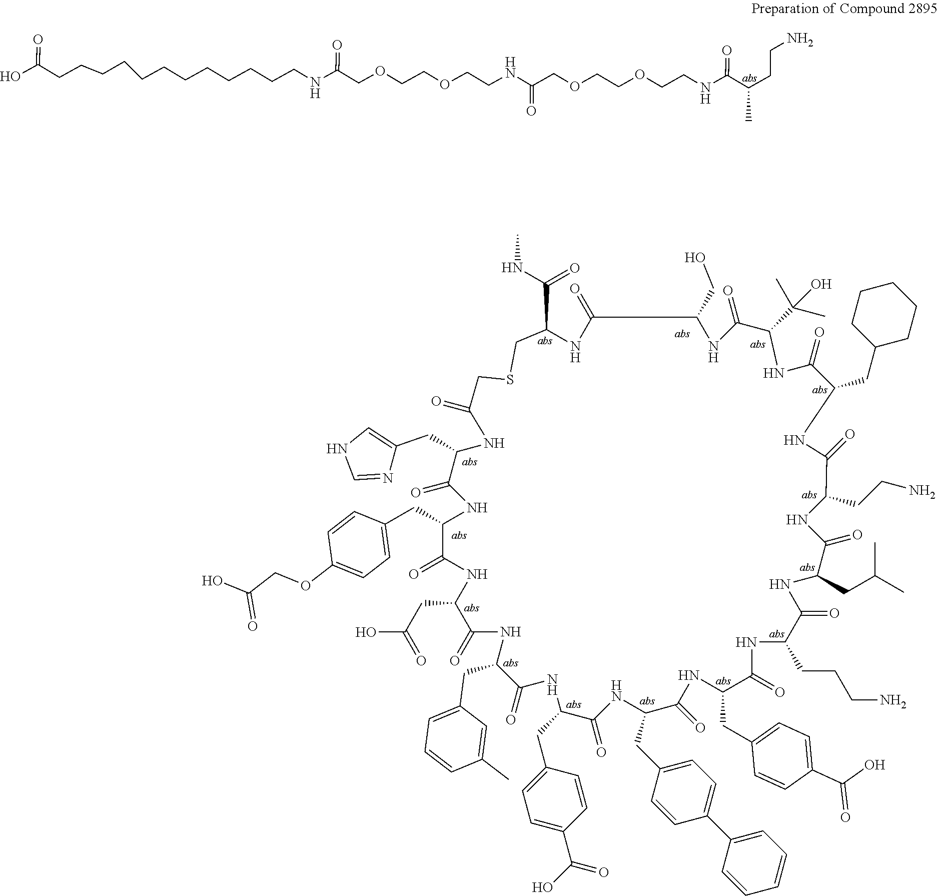

Compound 2895 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 16.5 mg, and its estimated purity by LCMS analysis was 91.9%. Analysis condition A: Retention time=1.51 min; ESI-MS(+) m/z [M+2H]²⁺: 1343.5.

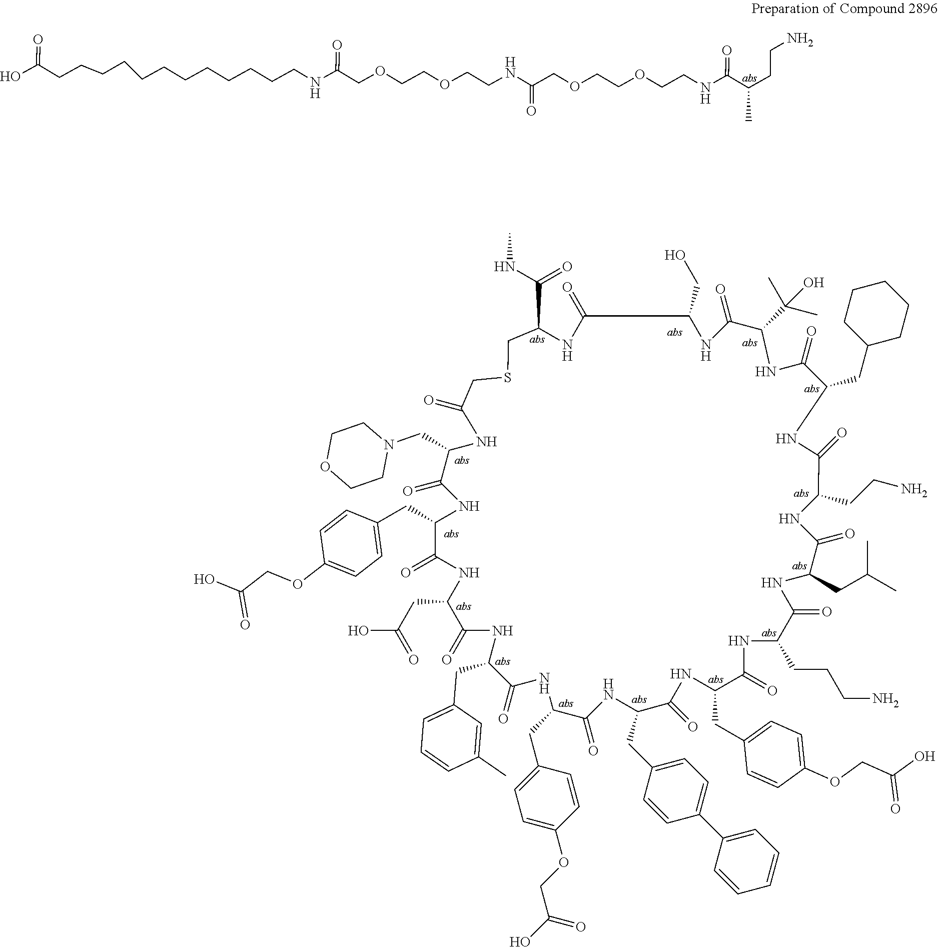

Compound 2896 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 15.8 mg, and its estimated purity by LCMS analysis was 98.6%. Analysis condition A: Retention time=1.64 min; ESI-MS(+) m/z [M+2H]²⁺: 1383.3.

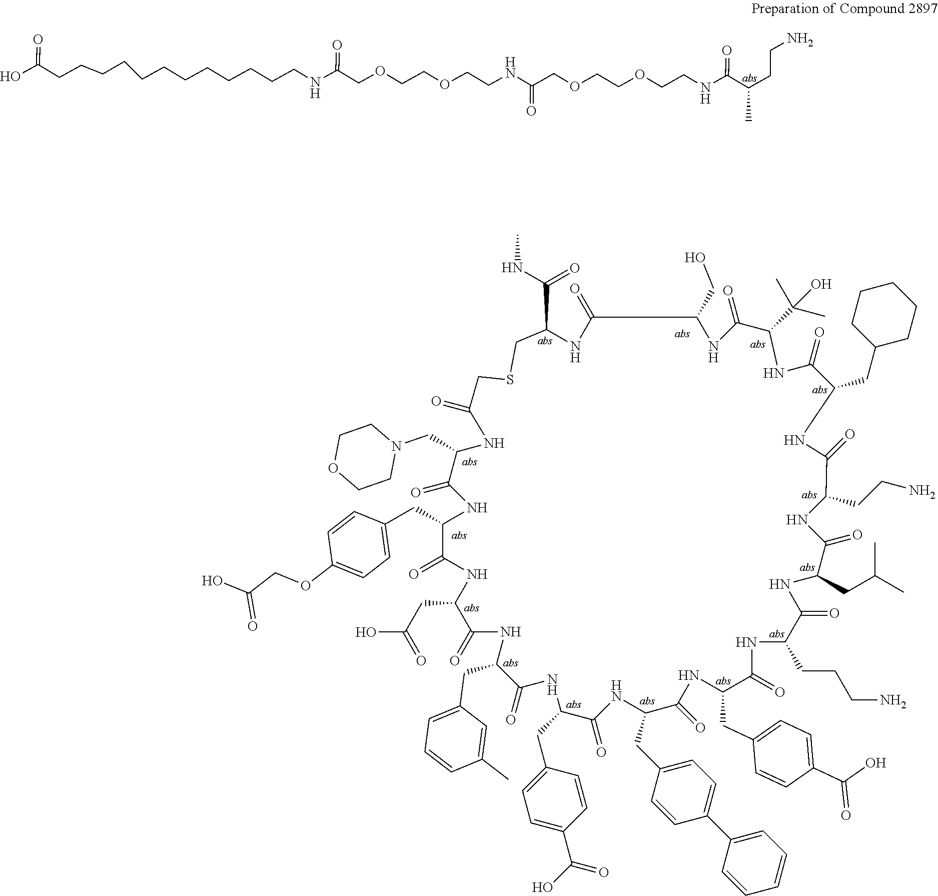

Compound 2897 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 2-minute hold at 100% B; Flow Rate: 40 mL/min; Column Temperature: 25 C. Fraction collection was triggered by MS signals. Fractions containing the desired product were combined and dried via centrifugal evaporation. The yield of the product was 13.2 mg, and its estimated purity by LCMS analysis was 100%. Analysis condition A: Retention time=1.5 min; ESI-MS(+) m/z [M+2H]²⁺: 1353.1.

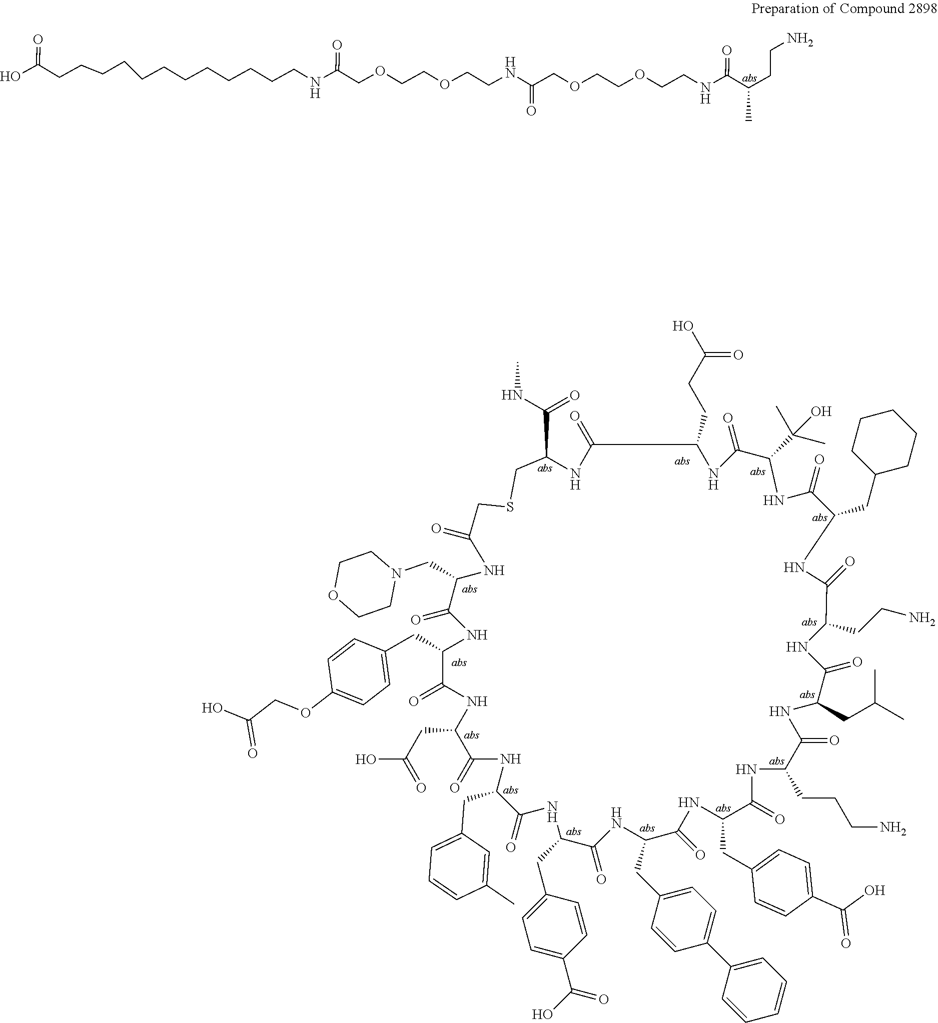

Compound 2898 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 13.3 mg, and its estimated purity by LCMS analysis was 97.1%. Analysis condition B: Retention time=1.63 min; ESI-MS(+) m/z [M+2H]²⁺: 1374.

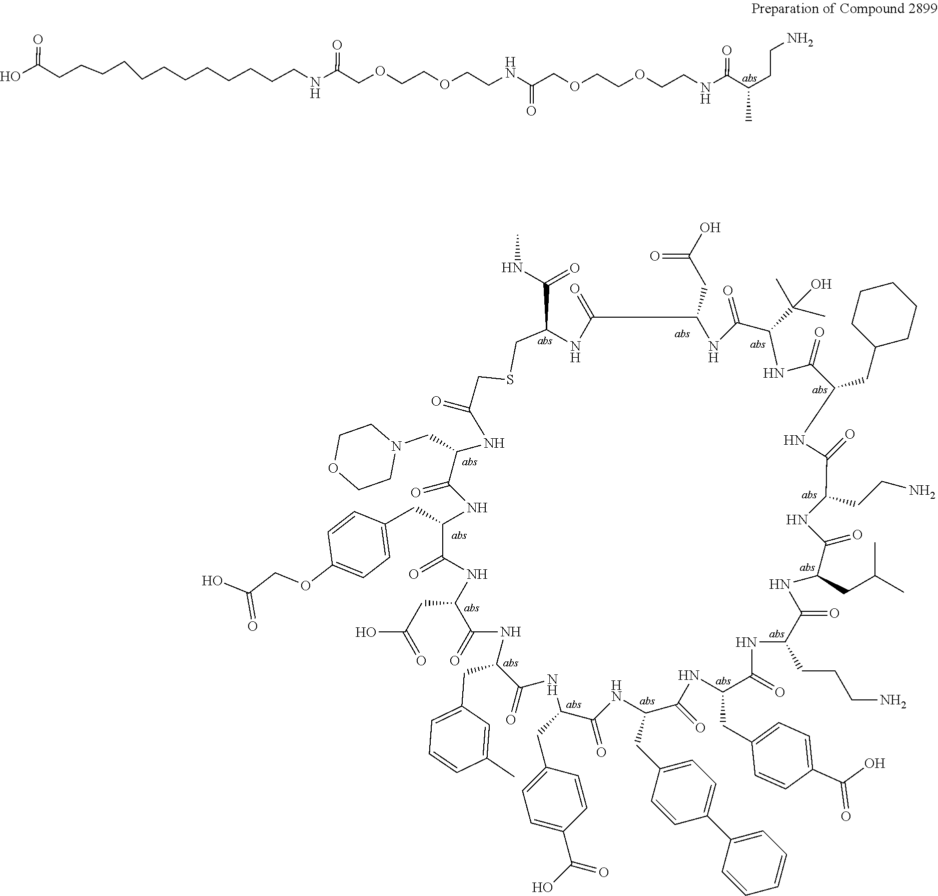

Compound 2899 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 21% B, 21-61% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 13% B, 13-53% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 16.9 mg, and its estimated purity by LCMS analysis was 98.6%. Analysis condition A: Retention time=1.42 min; ESI-MS(+) m/z [M+2H]²⁺: 1367.2.

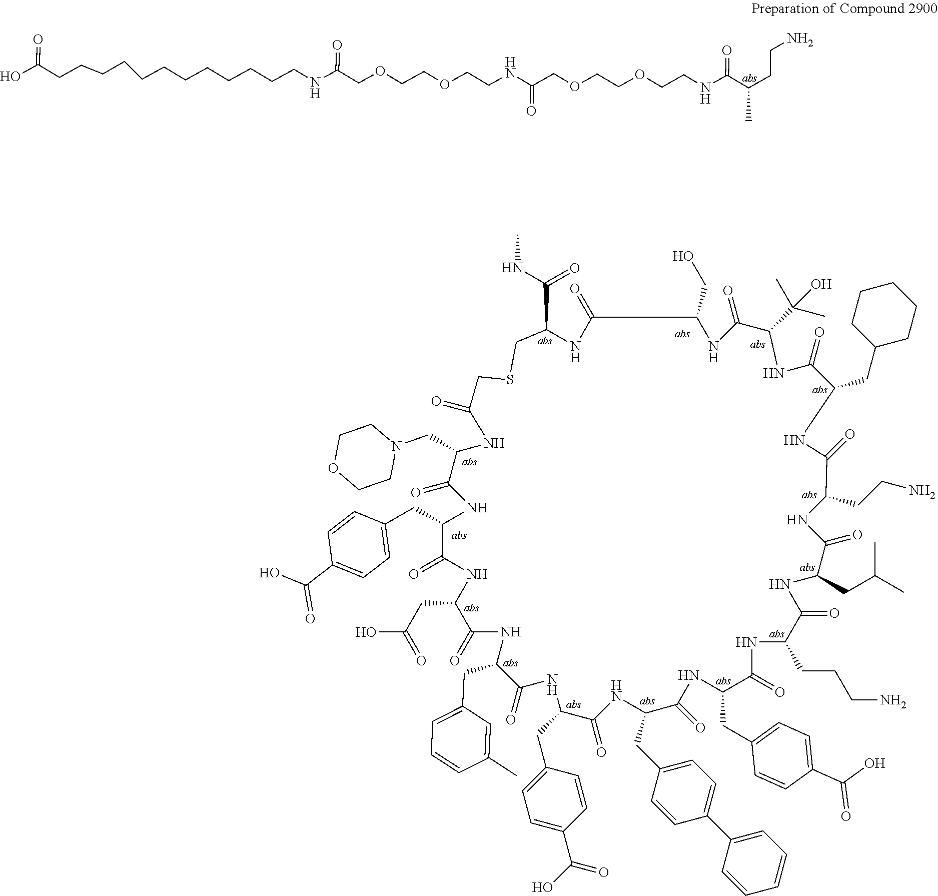

Compound 2900 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 15% B, 15-60% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 6.5 mg, and its estimated purity by LCMS analysis was 95.1%. Analysis condition B: Retention time=1.79 min; ESI-MS(+) m/z [M+3H]³⁺: 892.1.

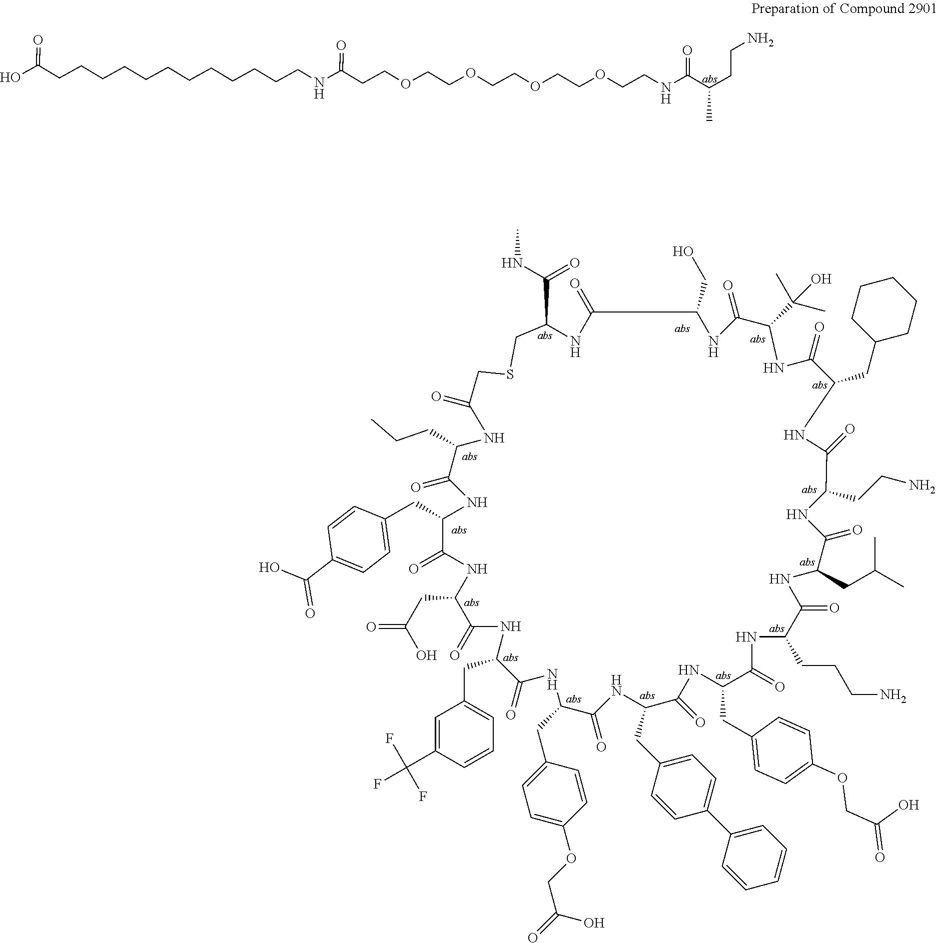

Compound 2901 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 18.6 mg, and its estimated purity by LCMS analysis was 97.9%. Analysis condition A: Retention time=1.67 min; ESI-MS(+) m/z [M+2H]²⁺: 1345.2.

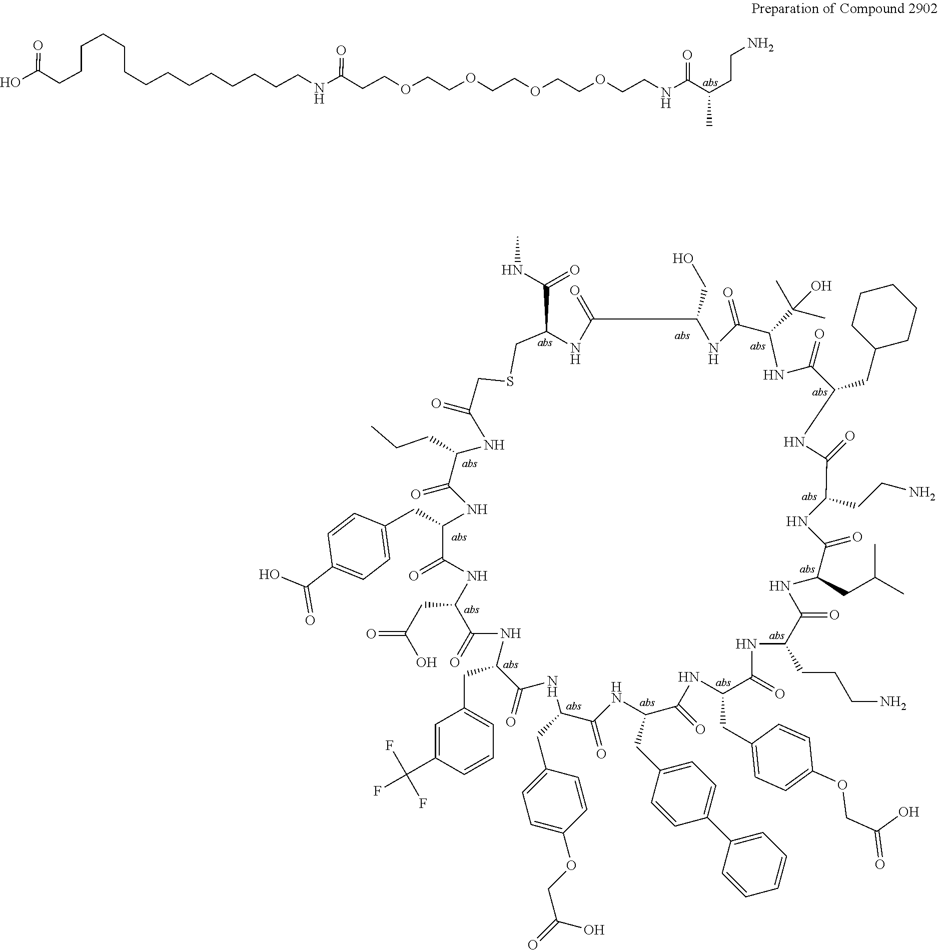

Compound 2902 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 21% B, 21-61% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 16.5 mg, and its estimated purity by LCMS analysis was 90.1%. Analysis condition B: Retention time=2 min; ESI-MS(+) m/z [M+2H]²⁺: 1359.4.

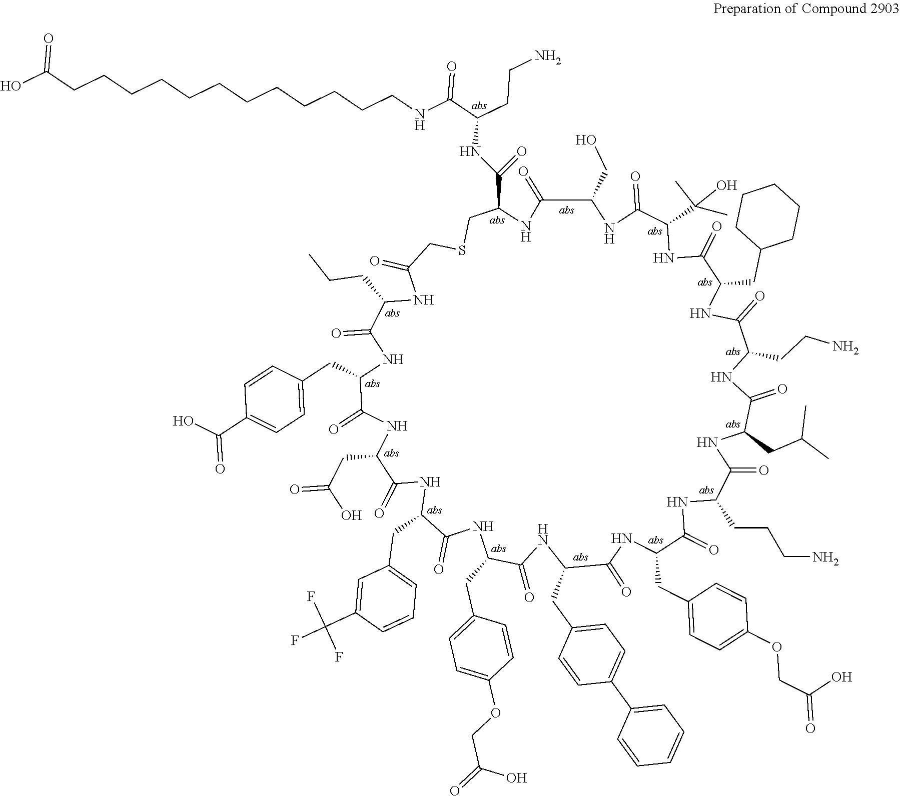 [SMILES: CCC[C@@H]1NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC1=O]

Compound 2903 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 25% B, 25-65% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 18% B, 18-58% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 13.5 mg, and its estimated purity by LCMS analysis was 100%. Analysis condition A: Retention time=1.66 min; ESI-MS(+) m/z [M+2H]²⁺: 1221.1.

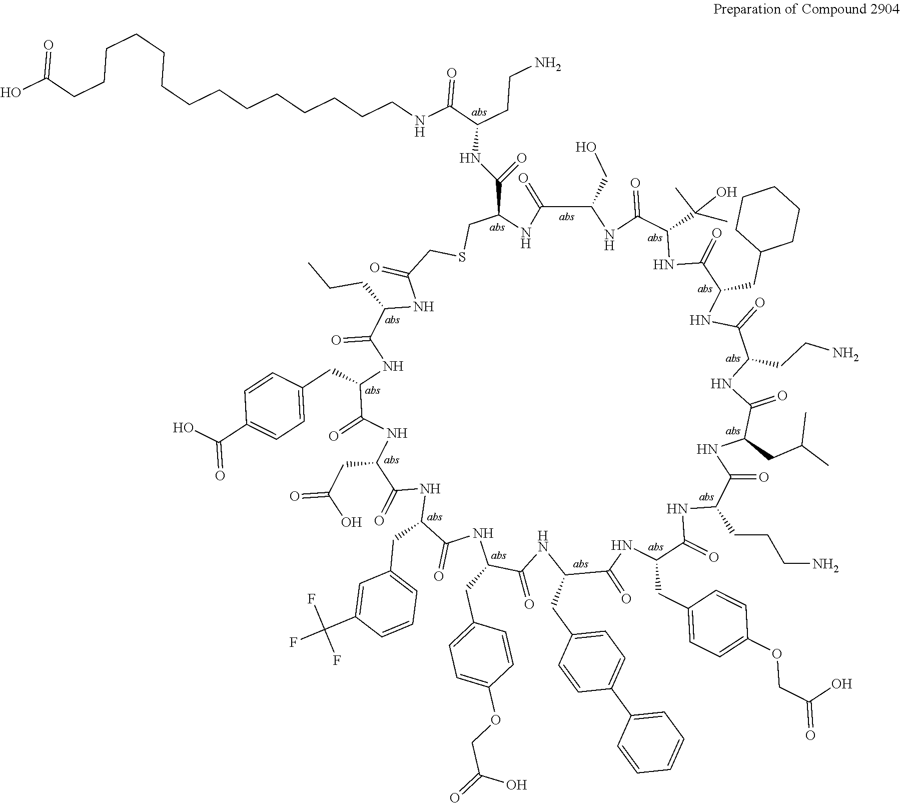 [SMILES: CCC[C@@H]1NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC1=O]

Compound 2904 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 28% B, 28-68% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 17.3 mg, and its estimated purity by LCMS analysis was 99.4%. Analysis condition B: Retention time=2.02 min; ESI-MS(+) m/z [M+2H]²⁺: 824.2.

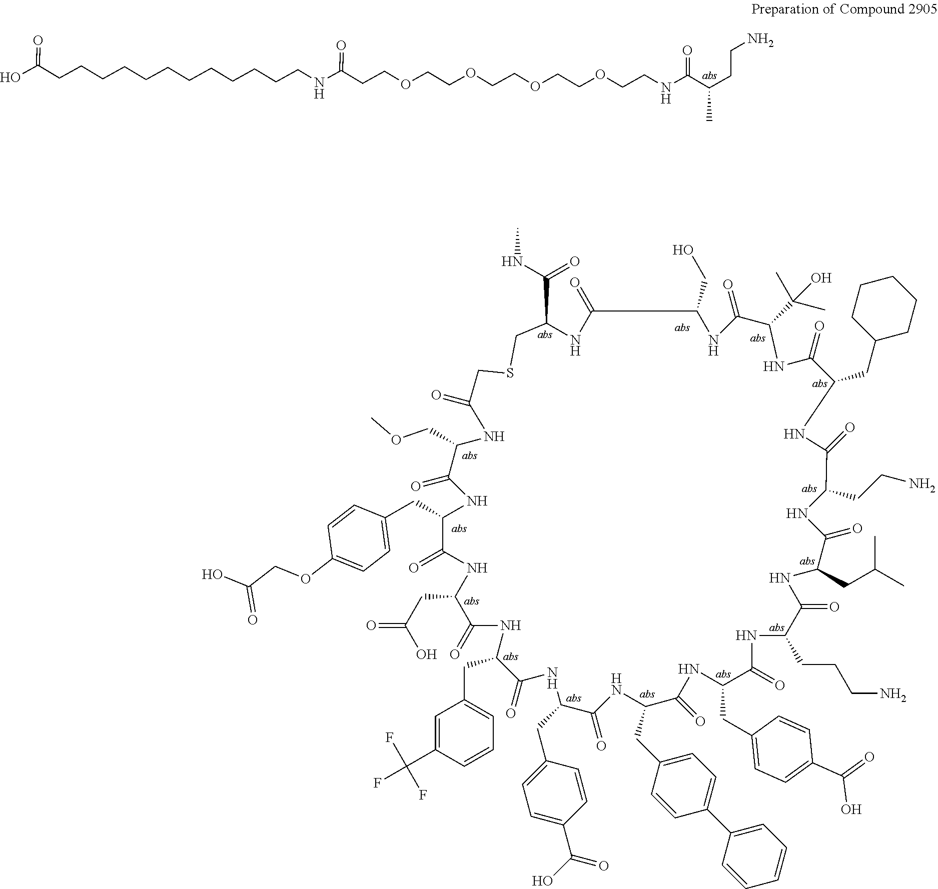

Compound 2905 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 26% B, 26-66% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 21.1 mg, and its estimated purity by LCMS analysis was 97.6%. Analysis condition B: Retention time=1.93 min; ESI-MS(+) m/z [M+2H]²⁺: 888.

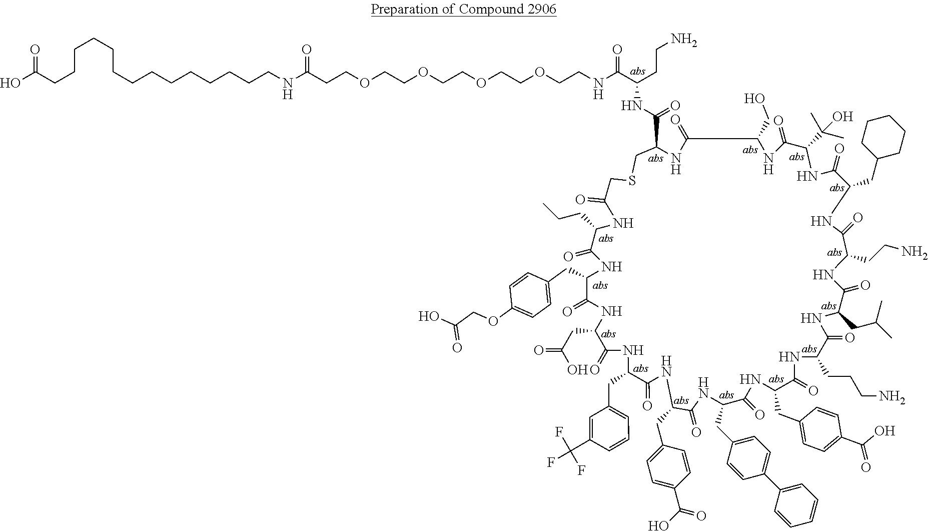 [SMILES: CCC[C@@H]1NC(=O)CSC[C@@H](C(=O)N[C@@H](CCN)C(=O)NCCOCCOCCOCCOCCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2906 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 30% B, 30-70% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 6.5 mg, and its estimated purity by LCMS analysis was 91.1%. Analysis condition A: Retention time=1.69 min; ESI-MS(+) m/z [M+2H]²⁺: 1345.1.

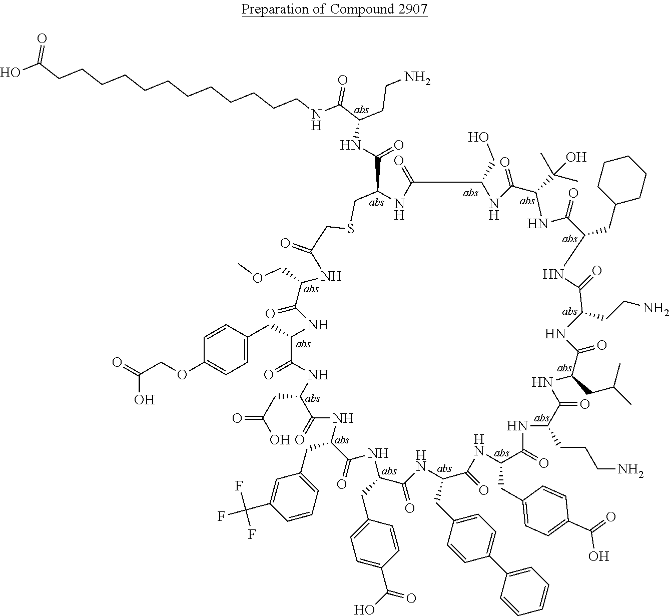 [SMILES: COC[C@@H]1NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2907 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 9.6 mg, and its estimated purity by LCMS analysis was 95.5%. Analysis condition A: Retention time=1.56 min; ESI-MS(+) m/z [M+2H]²⁺: 1207.9.

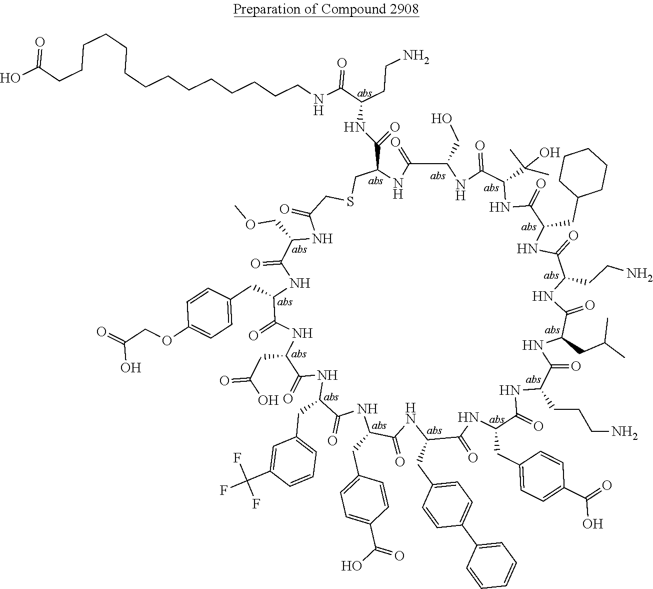 [SMILES: COC[C@@H]1NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2908 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 26% B, 26-66% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 22% B, 22-62% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 5 mg, and its estimated purity by LCMS analysis was 90%. Analysis condition A: Retention time=1.66 min; ESI-MS(+) m/z [M+2H]²⁺: 1221.2.

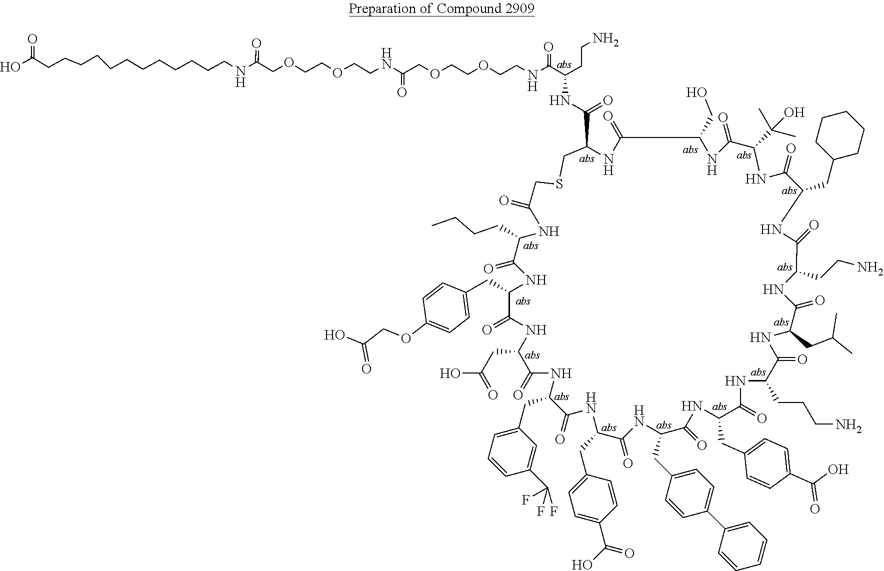 [SMILES: CCCC[C@@H]1NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2909 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe(3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 26% B, 26-66% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 25.1 mg, and its estimated purity by LCMS analysis was 97.4%. Analysis condition B: Retention time=1.95 min; ESI-MS(+) m/z [M+3H]³⁺: 906.1.

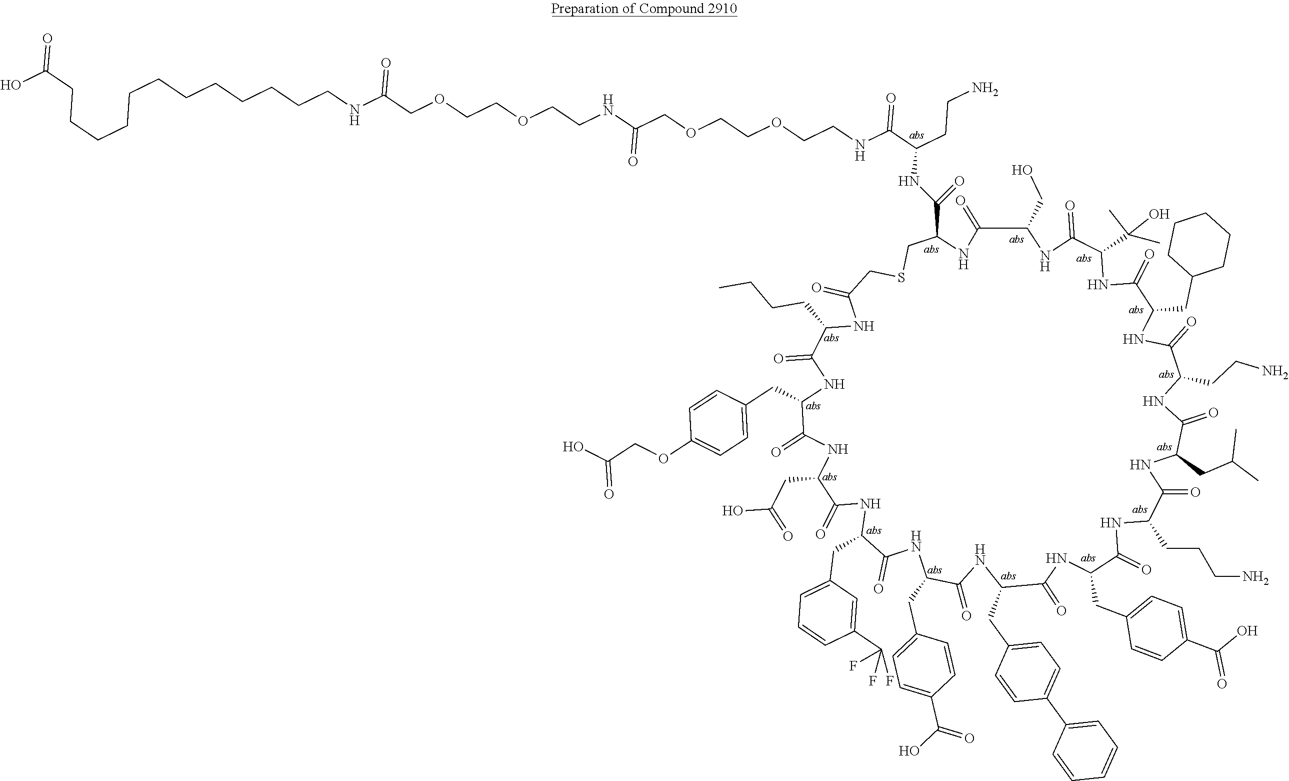 [SMILES: CCCC[C@@H]1NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2910 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-Coupling Manual Addition Procedure A” was followed with Fmoc-Phe (3-CF₃)—OH, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 31% B, 31-71% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 11.1 mg, and its estimated purity by LCMS analysis was 100%. Analysis condition A: Retention time=1.71 min; ESI-MS(+) m/z [M+2H]²⁺: 1373.

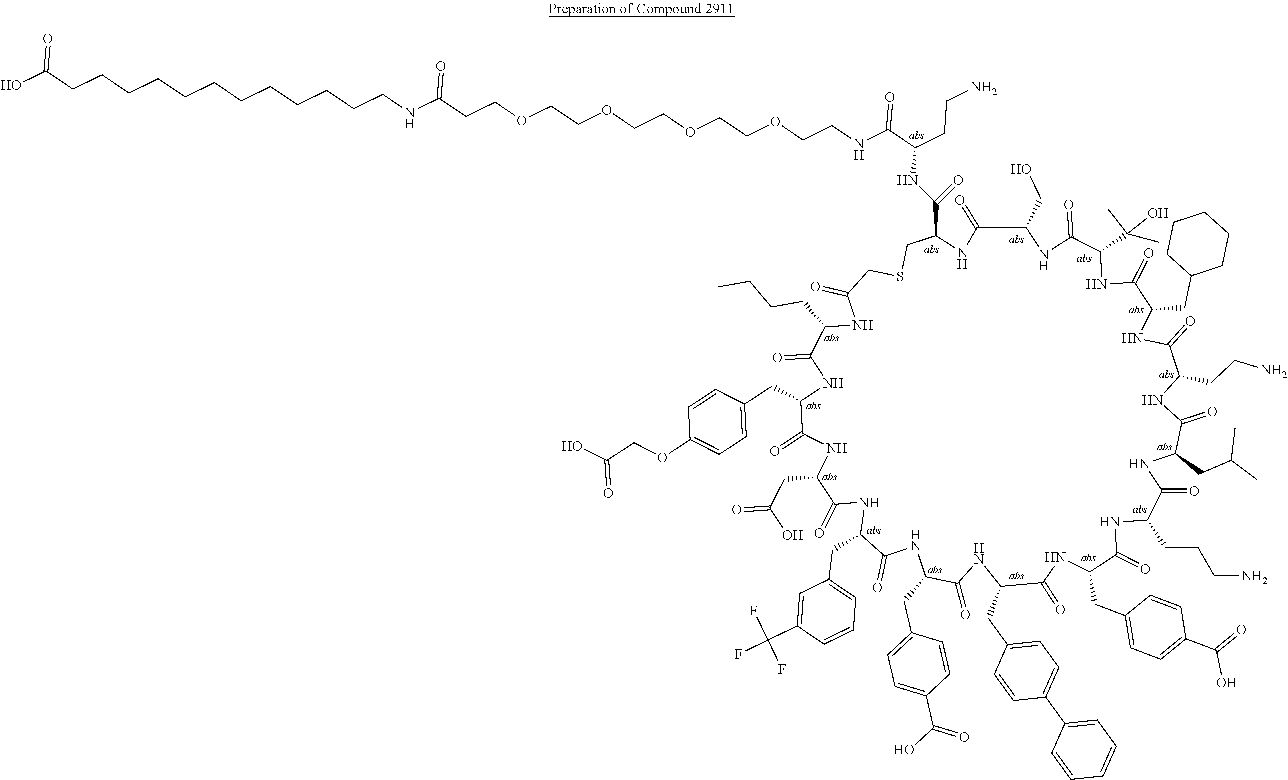 [SMILES: CCCC[C@@H]1NC(=O)CSC[C@@H](C(=O)N[C@@H](CCN)C(=O)NCCOCCOCCOCCOCCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2911 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 28% B, 28-68% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 22.2 mg, and its estimated purity by LCMS analysis was 93.9%. Analysis condition A: Retention time=1.64 min; ESI-MS(+) m/z [M+2H]²⁺: 1337.

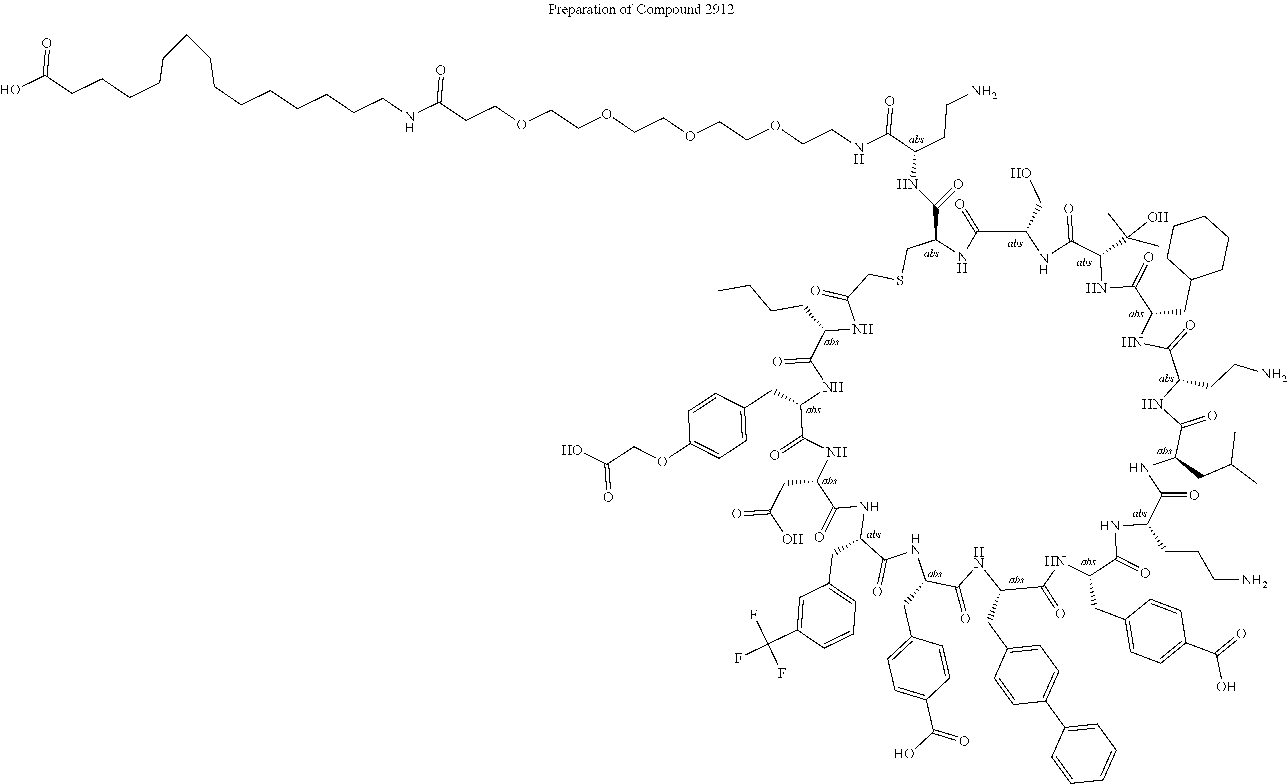 [SMILES: CCCC[C@@H]1NC(=O)CSC[C@@H](C(=O)N[C@@H](CCN)C(=O)NCCOCCOCCOCCOCCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2912 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 31% B, 31-71% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 21% B, 21-61% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 6.9 mg, and its estimated purity by LCMS analysis was 91.8%. Analysis condition B: Retention time=1.97 min; ESI-MS(+) m/z [M+2H]²⁺: 1351.2.

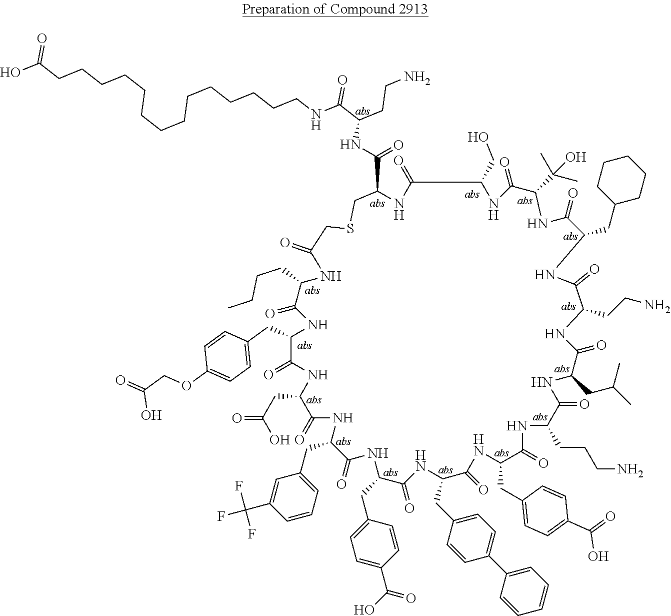 [SMILES: CCCC[C@@H]1NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2913 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 29% B, 29-69% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 13.5 mg, and its estimated purity by LCMS analysis was 98.1%. Analysis condition A: Retention time=1.86 min; ESI-MS(+) m/z [M+H]⁺: 2453.9.

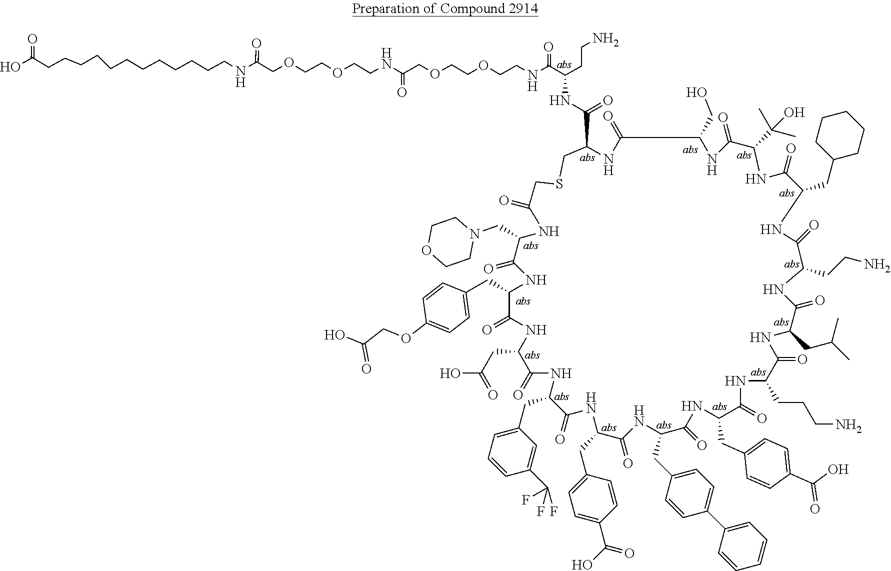 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 2914 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 16% B, 16-56% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 7.6 mg, and its estimated purity by LCMS analysis was 99.1%. Analysis condition B: Retention time=1.87 min; ESI-MS(+) m/z [M+2H]²⁺: 1380.3.

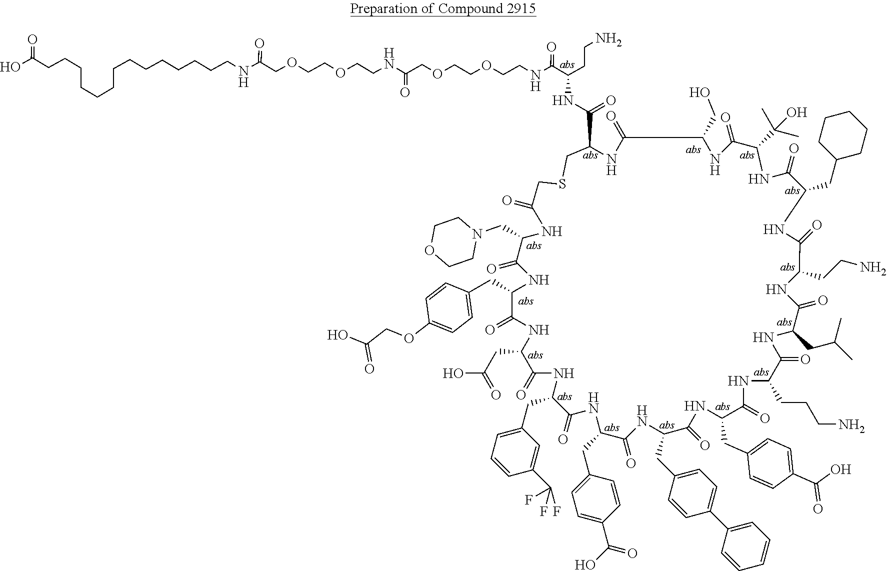 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 2915 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 26% B, 26-66% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 13.1 mg, and its estimated purity by LCMS analysis was 95.1%. Analysis condition B: Retention time=1.95 min; ESI-MS(+) m/z [M+2H]²⁺: 930.1.

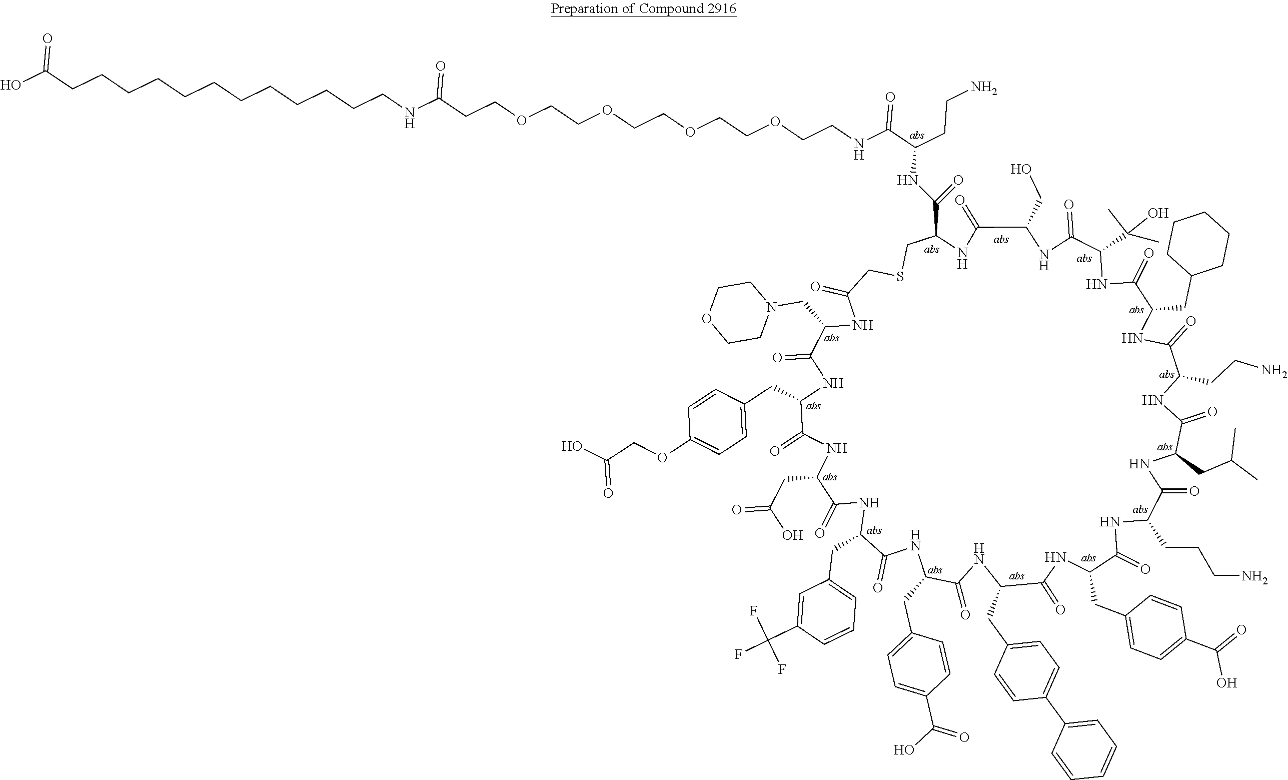 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCCOCCOCCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 2916 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 19% B, 19-59% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 9.8 mg, and its estimated purity by LCMS analysis was 97.3%. Analysis condition A: Retention time=1.59 min; ESI-MS(+) m/z [M+2H]²⁺: 1359.

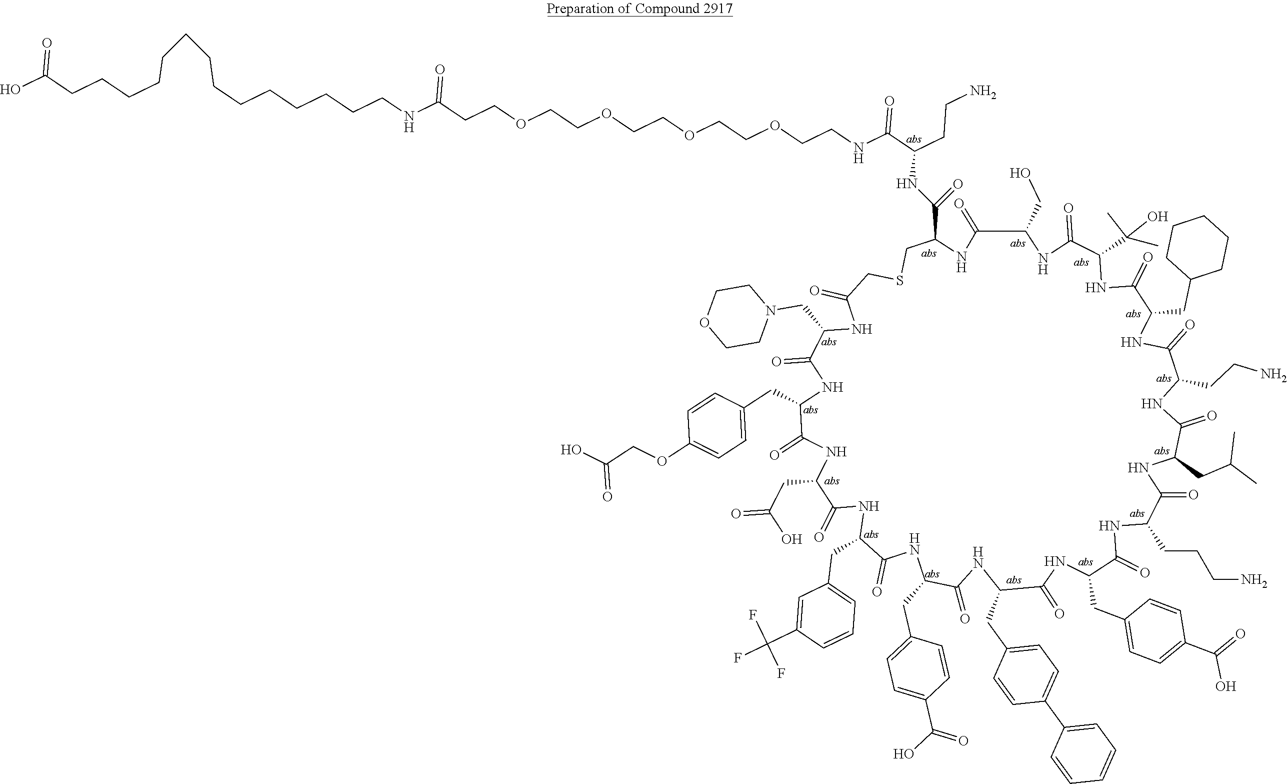 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCCOCCOCCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 2917 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 27% B, 27-67% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 12.6 mg, and its estimated purity by LCMS analysis was 97.6%. Analysis condition A: Retention time=1.77 min; ESI-MS(+) m/z [M+3H]³⁺: 915.2.

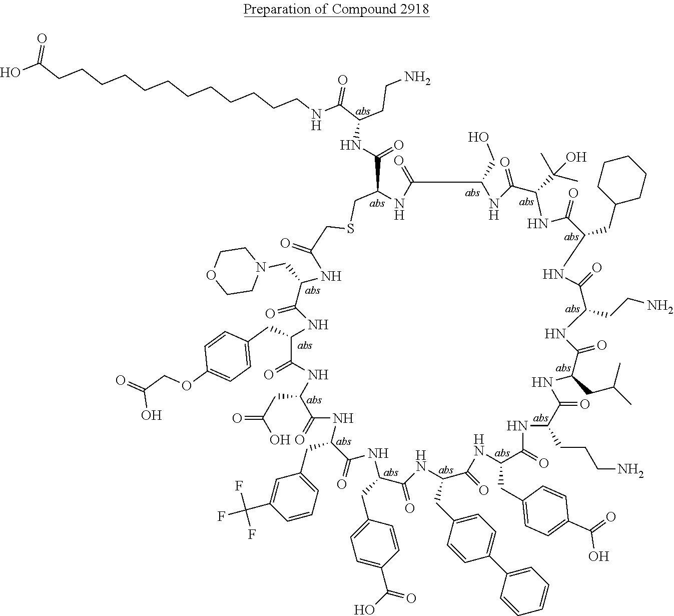 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 2918 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 17% B, 17-57% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 22% B, 22-62% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 5.2 mg, and its estimated purity by LCMS analysis was 91.8%. Analysis condition A: Retention time=1.58 min; ESI-MS(+) m/z [M+2H]²⁺: 1234.9.

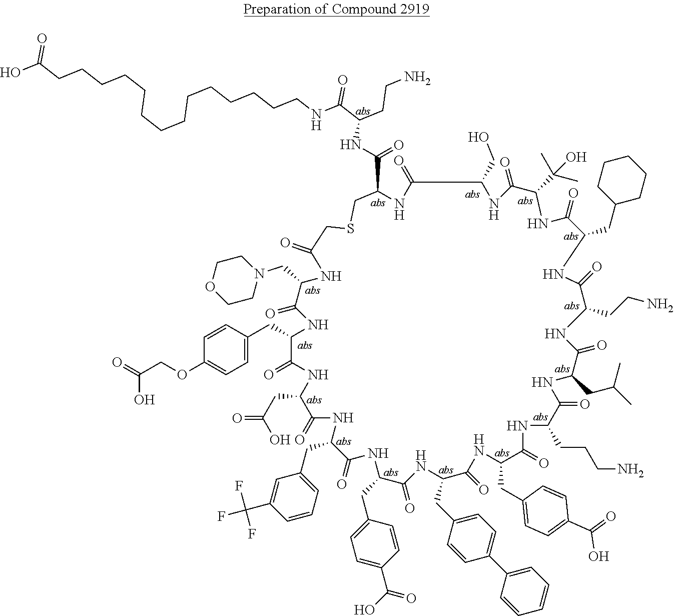 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 2919 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 20% B, 20-60% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 28% B, 28-68% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 4.1 mg, and its estimated purity by LCMS analysis was 93.5%. Analysis condition A: Retention time=1.67 min; ESI-MS(+) m/z [M+2H]²⁺: 1249.

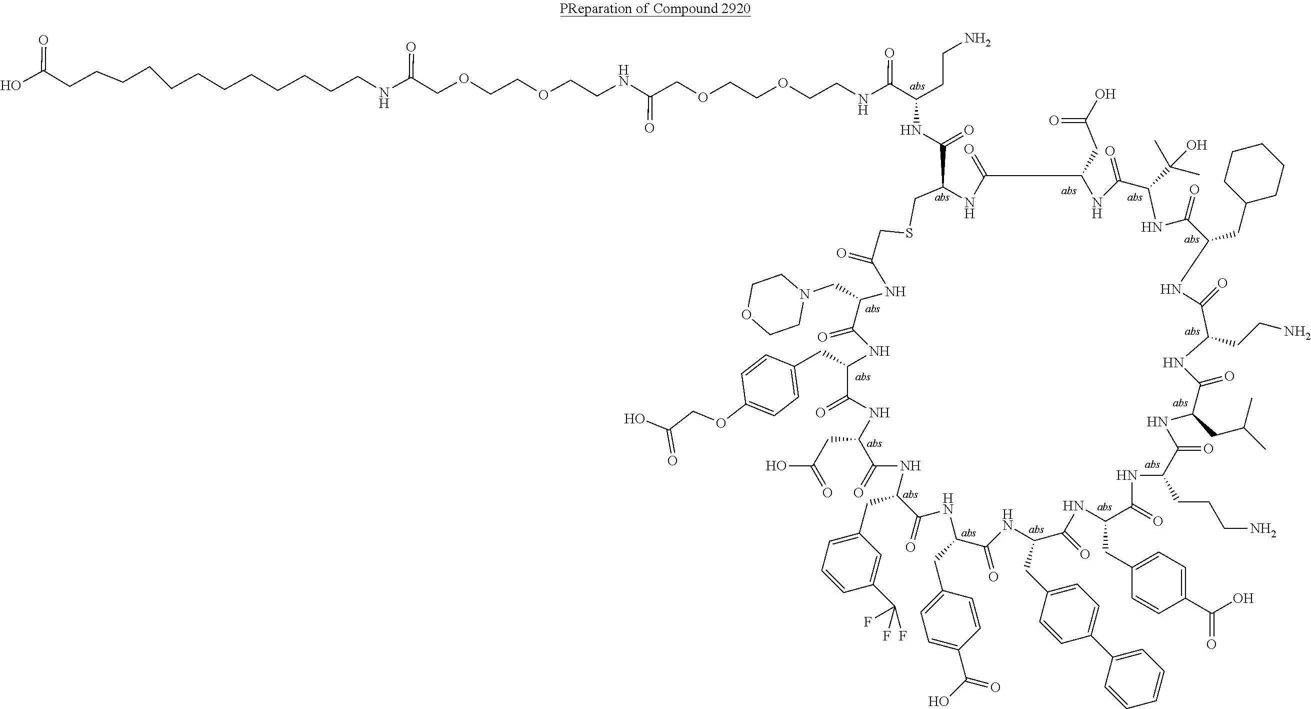 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC1=O]

Compound 2920 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 40 mL/min; Column Temperature: 25 C. Fraction collection was triggered by MS signals. Fractions containing the desired product were combined and dried via centrifugal evaporation. The yield of the product was 16.1 mg, and its estimated purity by LCMS analysis was 98.1%. Analysis condition B: Retention time=1.73 min; ESI-MS(+) m/z [M+2H]²⁺: 1393.9.

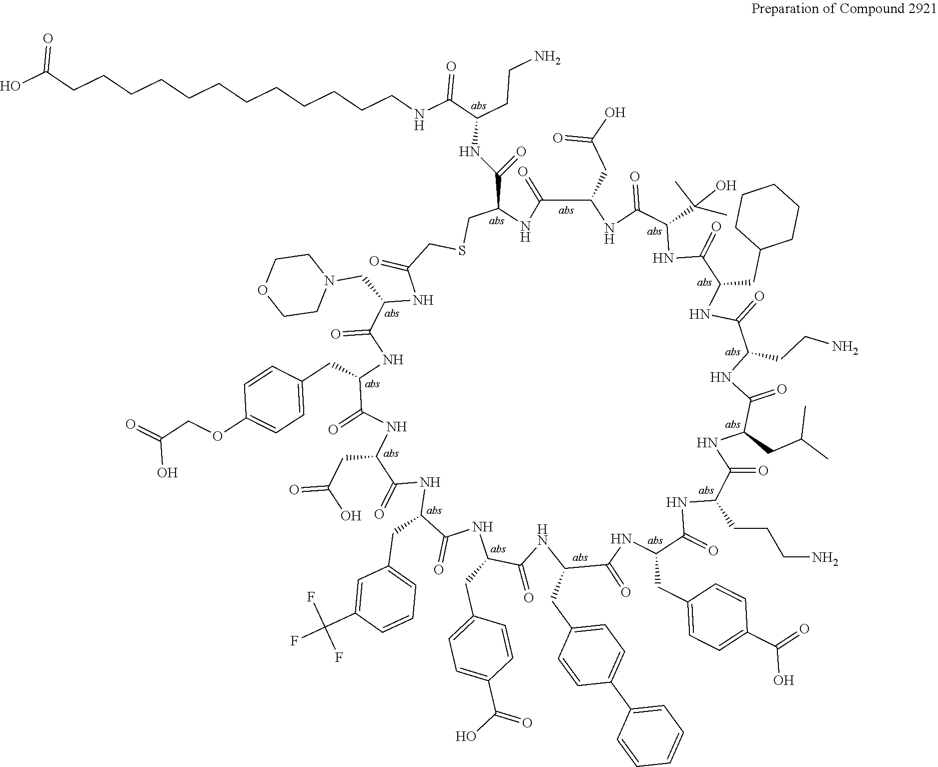 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC1=O]

Compound 2921 was prepared, using 2-Chlorotrityl resin pre-loaded with 13-((((9H-fluoren-9-yl) methoxy) carbonyl)amino)tridecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with ammonium acetate; Mobile Phase B: 95:5 acetonitrile:water with ammonium acetate; Gradient: a 0-minute hold at 14% B, 14-54% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 18.6 mg, and its estimated purity by LCMS analysis was 93.6%. Analysis condition B: Retention time=1.68 min; ESI-MS(+) m/z [M+2H]²⁺: 1249.1.

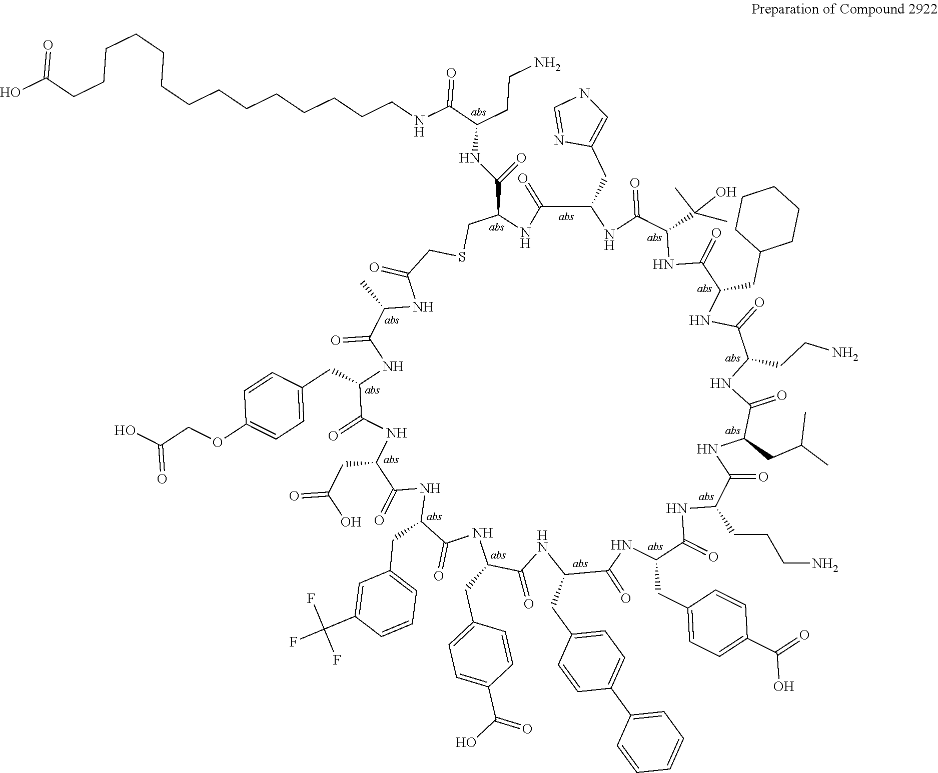 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Compound 2922 was prepared, using 2-Chlorotrityl resin pre-loaded with 15-((((9H-fluoren-9-yl) methoxy) carbonyl)amino) pentadecanoic acid on a 50 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Single-Coupling Manual Addition Procedure A” was followed with Fmoc-Phe (3-CF₃)—OH, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 23% B, 23-63% B over 20 minutes, then a 0-minute hold at 100% B; Flow Rate: 45 mL/min; Column Temperature: 25 C. Fraction collection was triggered by MS signals. Fractions containing the desired product were combined and dried via centrifugal evaporation. The yield of the product was 33.4 mg, and its estimated purity by LCMS analysis was 99%. Analysis condition B: Retention time=1.74 min; ESI-MS(+) m/z [M+3H]³⁺: 821.2.

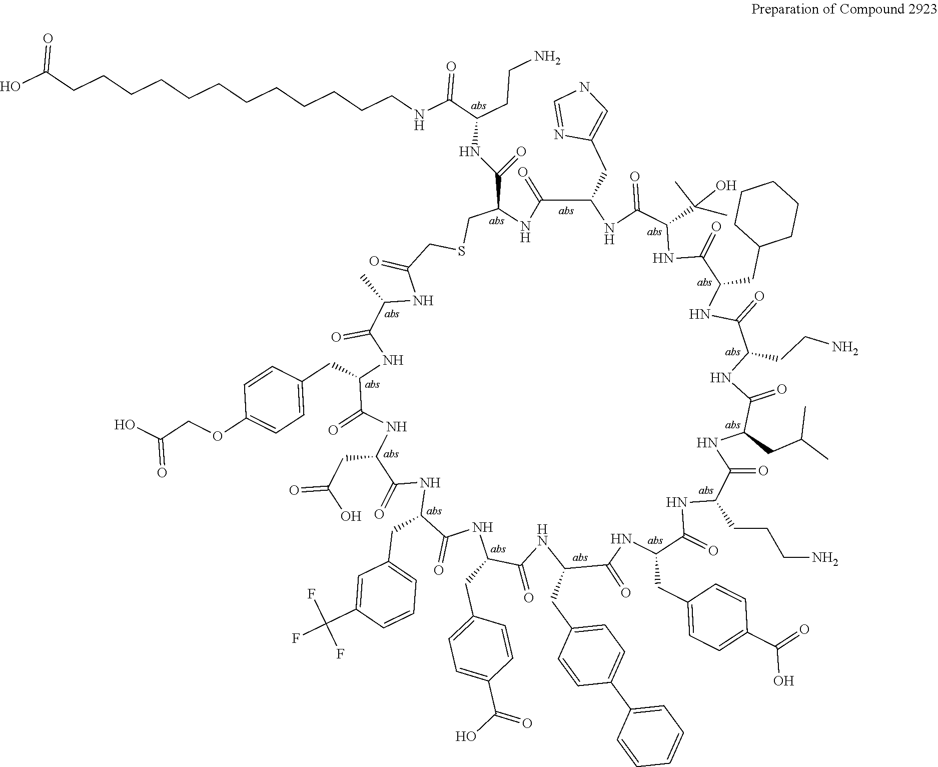 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Compound 2923 was prepared on a 50 μmol scale. The yield of the product was 3.1 mg, and its estimated purity by LCMS analysis was 96%. Analysis condition A: Retention time=1.56 min; ESI-MS(+) m/z [M+2H]²⁺: 1217.3.

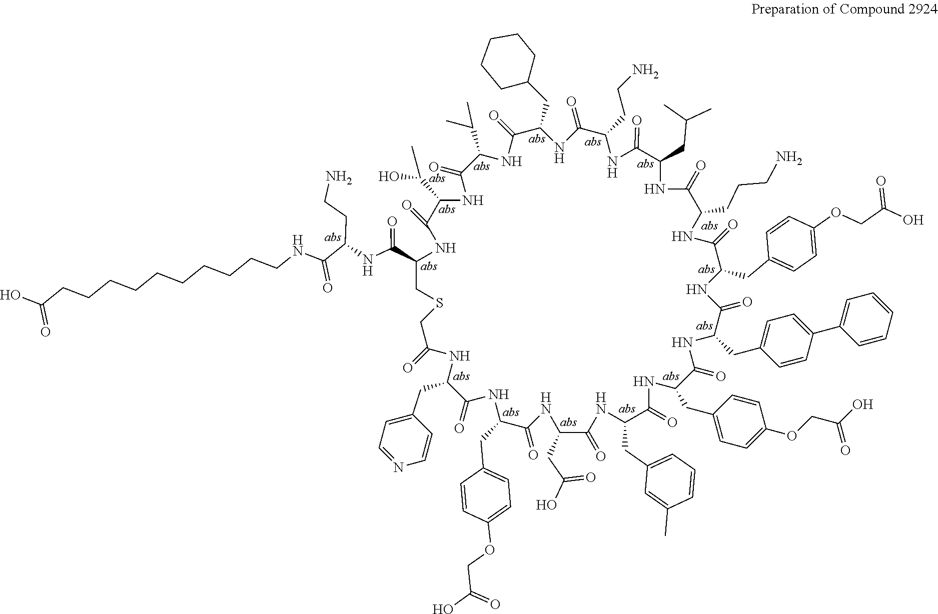 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2924 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 14% B, 14-54% B over 21 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 4.4 mg, and its estimated purity by LCMS analysis was 95.5%. Analysis condition 2: Retention time=1.64 min; ESI-MS(+) m/z [M+2H]²⁺: 1219.2.

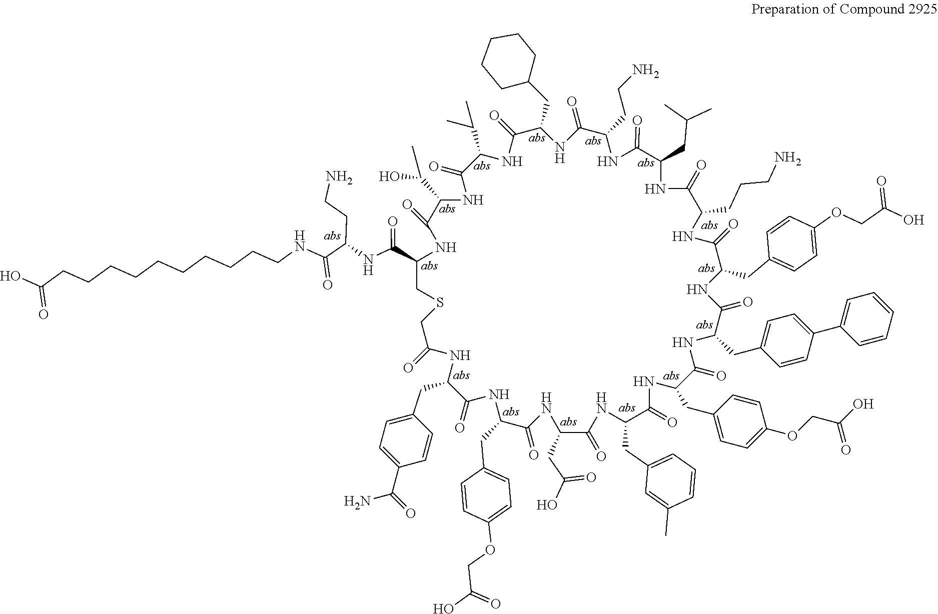 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCN)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2925 was prepared, using 2-Chlorotrityl resin pre-loaded with 11-((((9H-fluoren-9-yl) methoxy) carbonyl)amino) undecanoic acid on a 25 μmol scale, following the general synthetic sequence described for the preparation of Compound 1002 composed of the following general procedures: “Symphony X Resin-swelling procedure”, “Symphony X Single-coupling procedure”, or “Symphony X Double-coupling procedure”, “Symphony X Chloroacetic Anhydride coupling procedure”, “Symphony X Final rinse and dry procedure”, “Global Deprotection Method A”, “Cyclization Method A”. The crude material was purified via preparative LC/MS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 11% B, 11-51% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 5.5 mg, and its estimated purity by LCMS analysis was 91.1%. Analysis condition B: Retention time=1.72 min; ESI-MS(+) m/z [M+2H]²⁺: 1240.3.

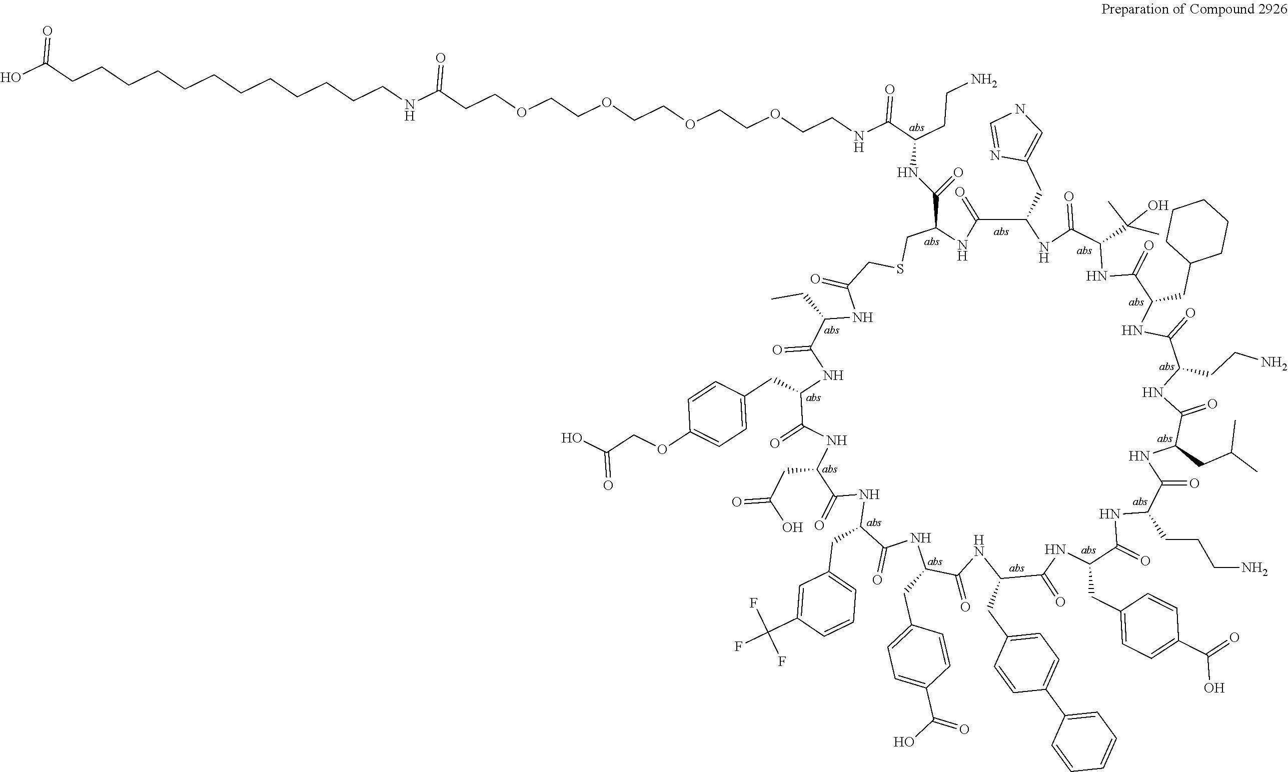 [SMILES: CC[C@@H]1NC(=O)CSC[C@@H](C(=O)N[C@@H](CCN)C(=O)NCCOCCOCCOCCOCCC(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2926 was prepared on a 50 μmol scale. The yield of the product was 6.6 mg, and its estimated purity by LCMS analysis was 100%. Analysis condition A: Retention time=1.63 min; ESI-MS(+) m/z [M+2H]²⁺: 1348.

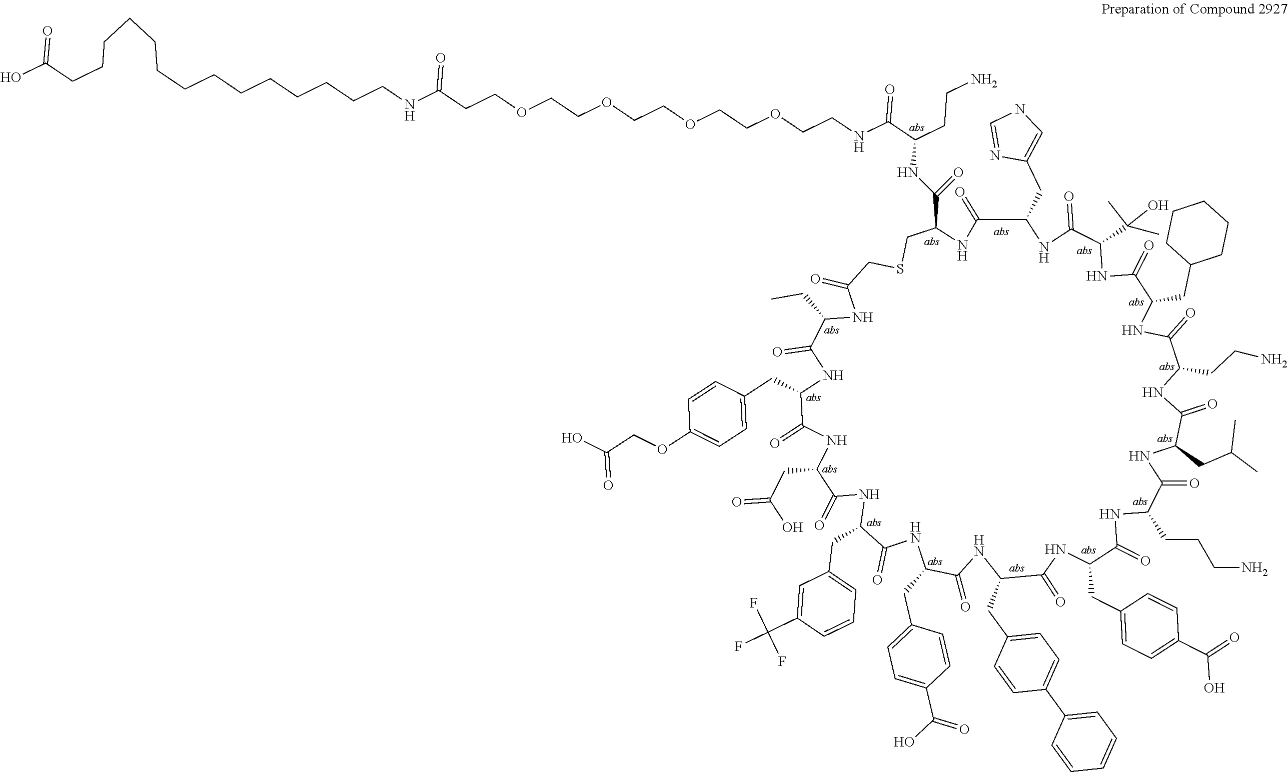 [SMILES: CC[C@@H]1NC(=O)CSC[C@@H](C(=O)N[C@@H](CCN)C(=O)NCCOCCOCCOCCOCCC(=O)NCCCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2927 was prepared on a 50 μmol scale. The yield of the product was 7.8 mg, and its estimated purity by LCMS analysis was 100%. Analysis condition A: Retention time=1.78 min; ESI-MS(+) m/z [M+2H]²⁺: 1362.

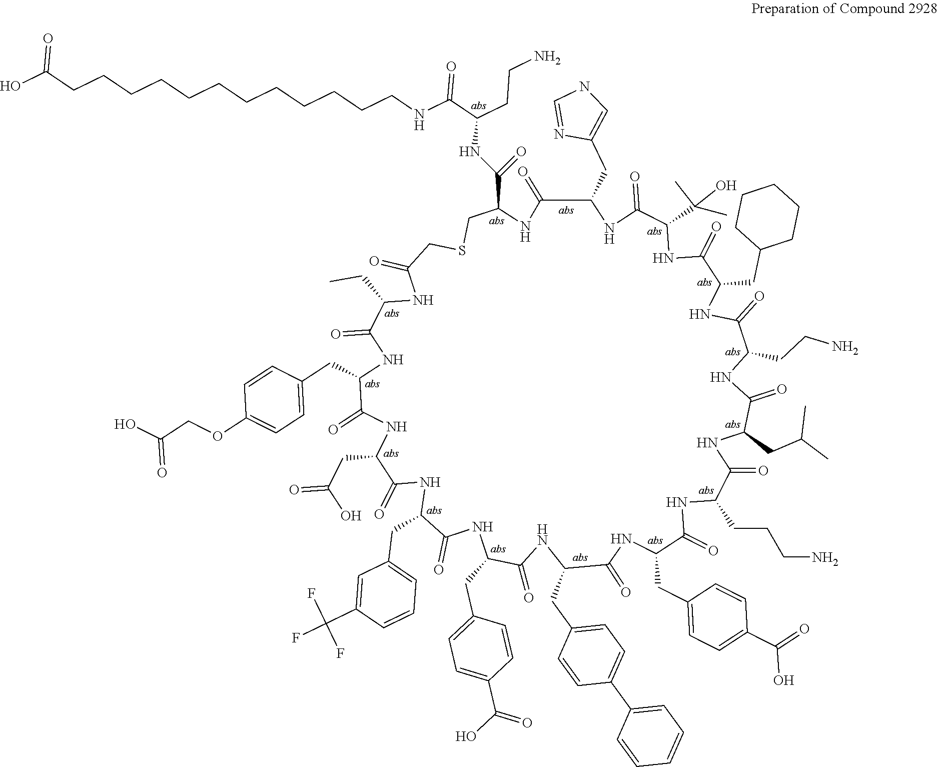 [SMILES: CC[C@@H]1NC(=O)CSC[C@@H](C(=O)N[C@@H](CCN)C(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2928 was prepared on a 50 μmol scale. The yield of the product was 3.6 mg, and its estimated purity by LCMS analysis was 96.1%. Analysis condition B: Retention time=1.72 min; ESI-MS(+) m/z [M+2H]²⁺: 1224.9.

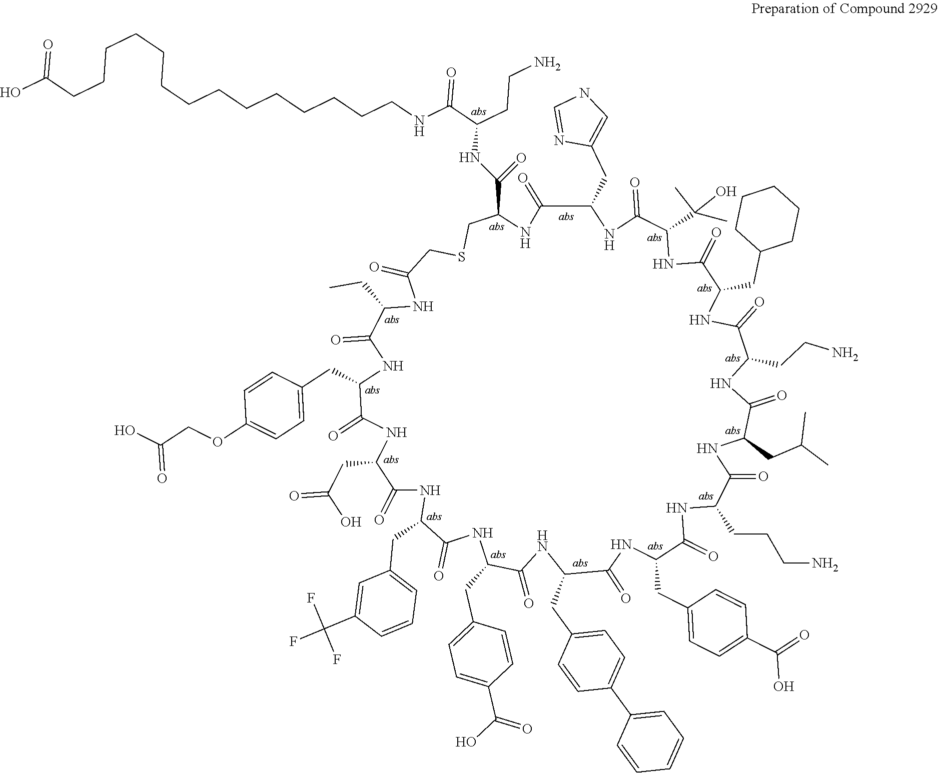 [SMILES: CC[C@@H]1NC(=O)CSC[C@@H](C(=O)N[C@@H](CCN)C(=O)NCCCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2929 was prepared on a 50 μmol scale. The yield of the product was 3.8 mg, and its estimated purity by LCMS analysis was 91.6%. Analysis condition B: Retention time=1.87 min; ESI-MS(+) m/z [M+3H]³⁺: 826.2.

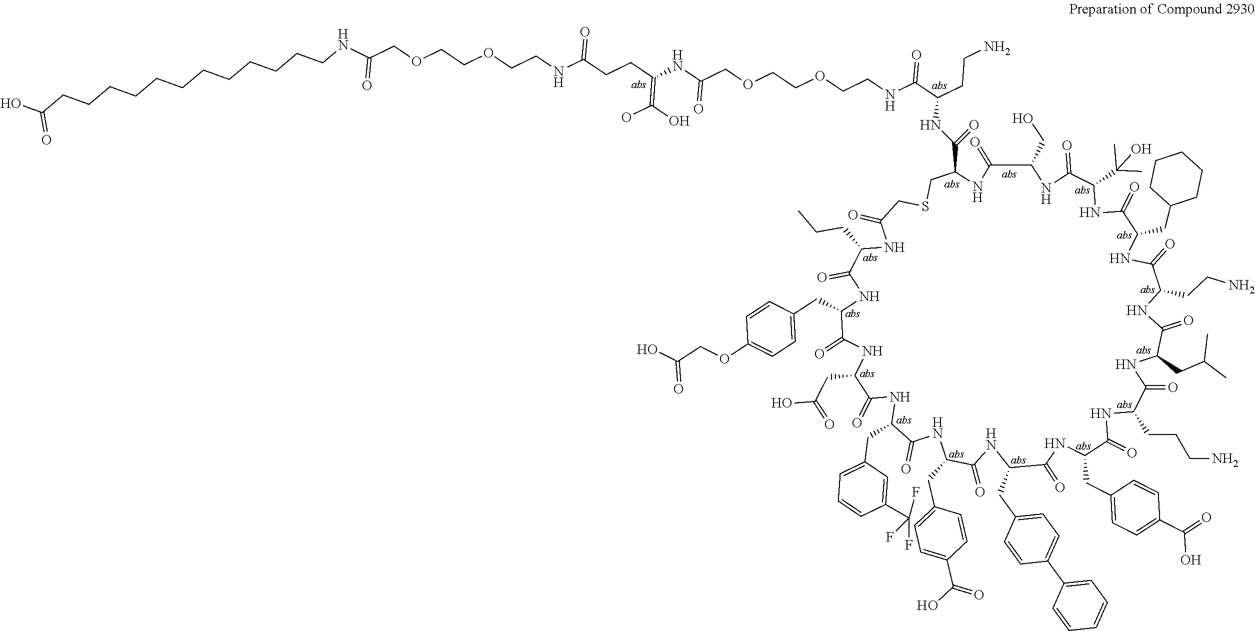

Compound 2930 was prepared on a 50 μmol scale. The yield of the product was 16.4 mg, and its estimated purity by LCMS analysis was 98.9%. Analysis condition A: Retention time=1.64 min; ESI-MS(+) m/z [M+2H]²⁺: 1415.9.

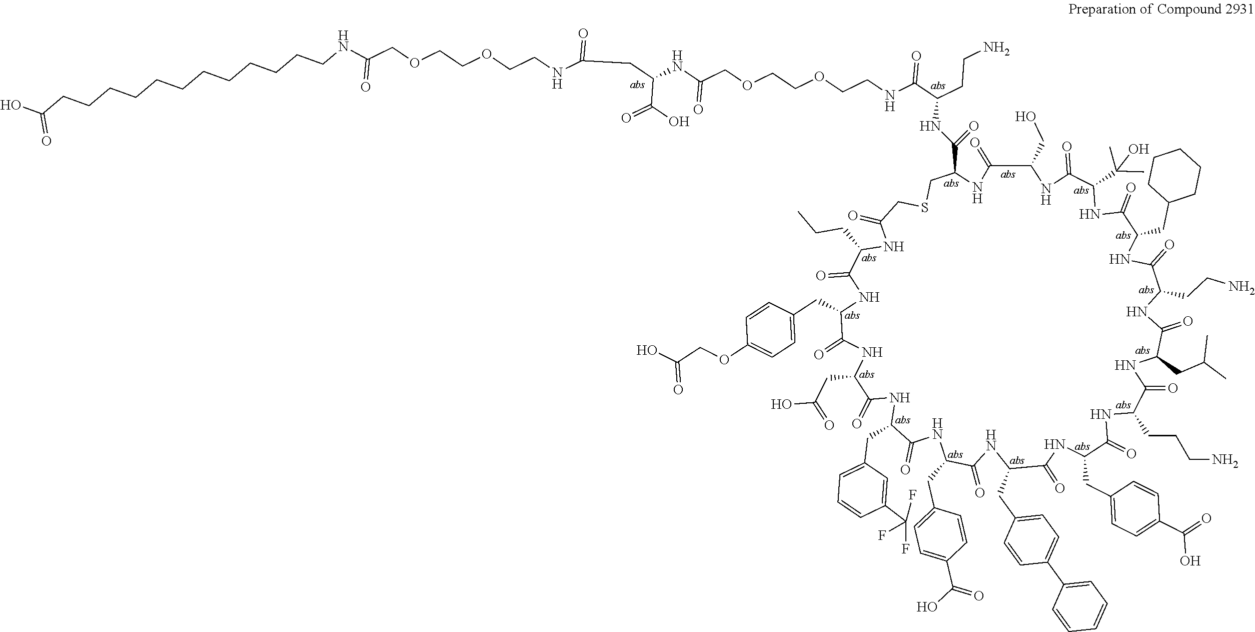 [SMILES: CCC[C@@H]1NC(=O)CSC[C@@H](C(=O)N[C@@H](CCN)C(=O)NCCOCCOCC(=O)N[C@@H](CC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2931 was prepared on a 50 μmol scale. The yield of the product was 13.7 mg, and its estimated purity by LCMS analysis was 95.8%. Analysis condition A: Retention time=1.63 min; ESI-MS(+) m/z [M+2H]²⁺: 1409.1.

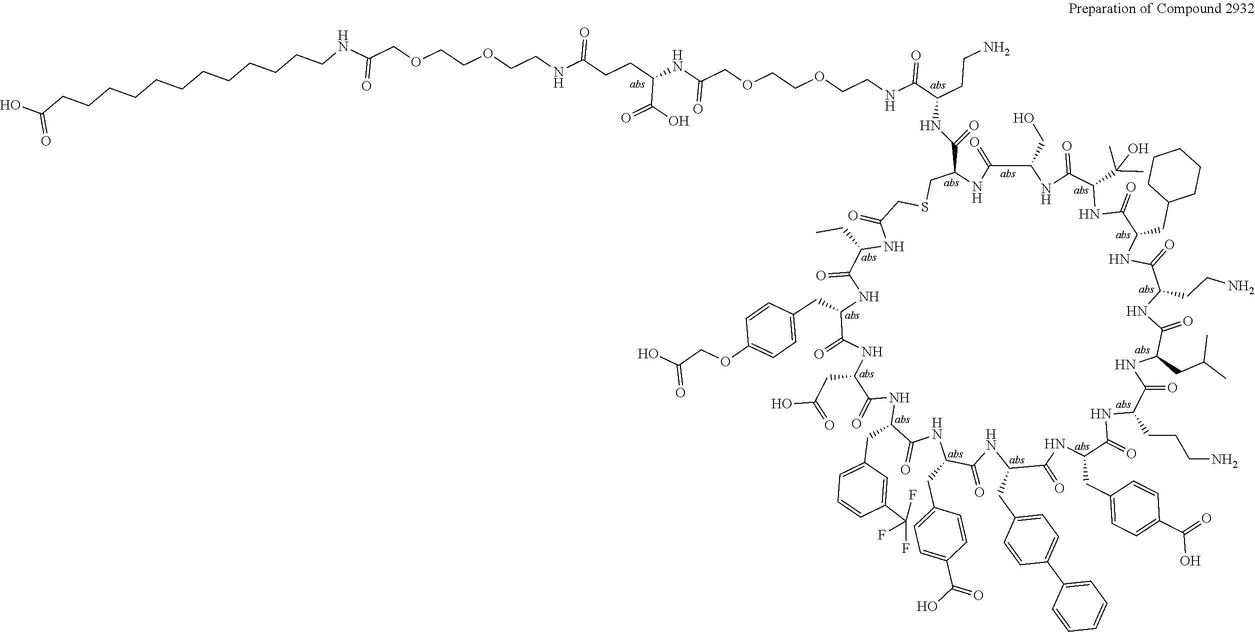 [SMILES: CC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2932 was prepared on a 50 μmol scale. The yield of the product was 25.8 mg, and its estimated purity by LCMS analysis was 93.4%. Analysis condition A: Retention time=1.6 min; ESI-MS(+) m/z [M+2H]²⁺: 1409.2.

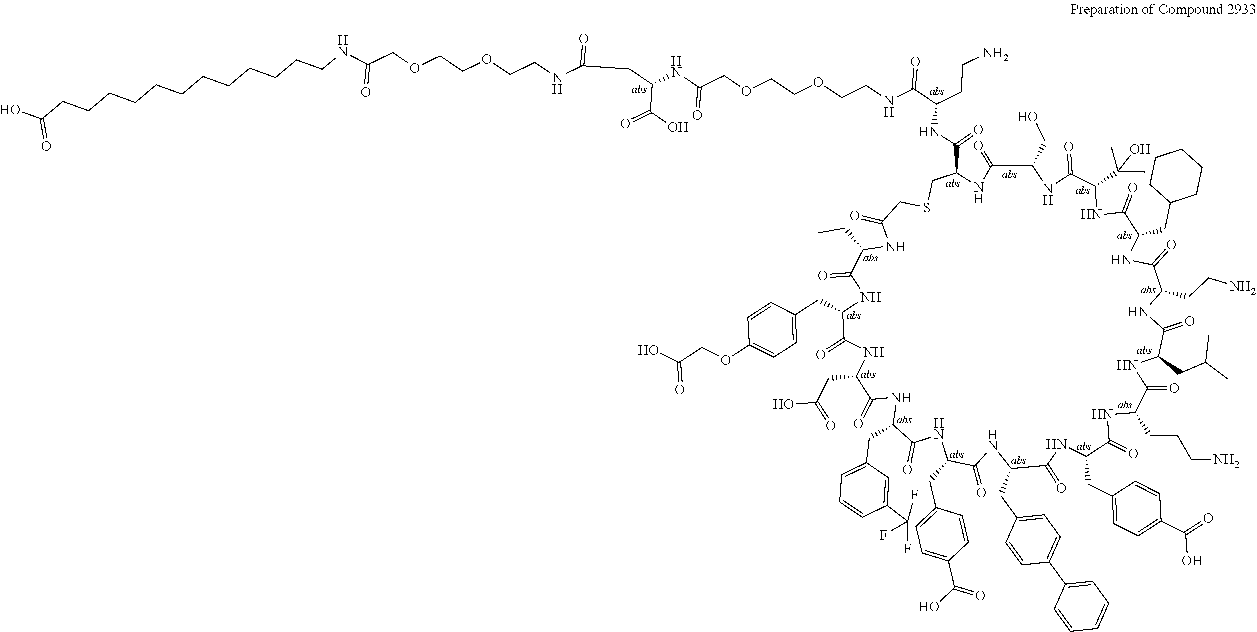 [SMILES: CC[C@@H]1NC(=O)CSC[C@@H](C(=O)N[C@@H](CCN)C(=O)NCCOCCOCC(=O)N[C@@H](CC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2933 was prepared on a 50 μmol scale. The yield of the product was 24.9 mg, and its estimated purity by LCMS analysis was 100%. Analysis condition B: Retention time=1.85 min; ESI-MS(+) m/z [M+2H]²⁺: 1402.1.

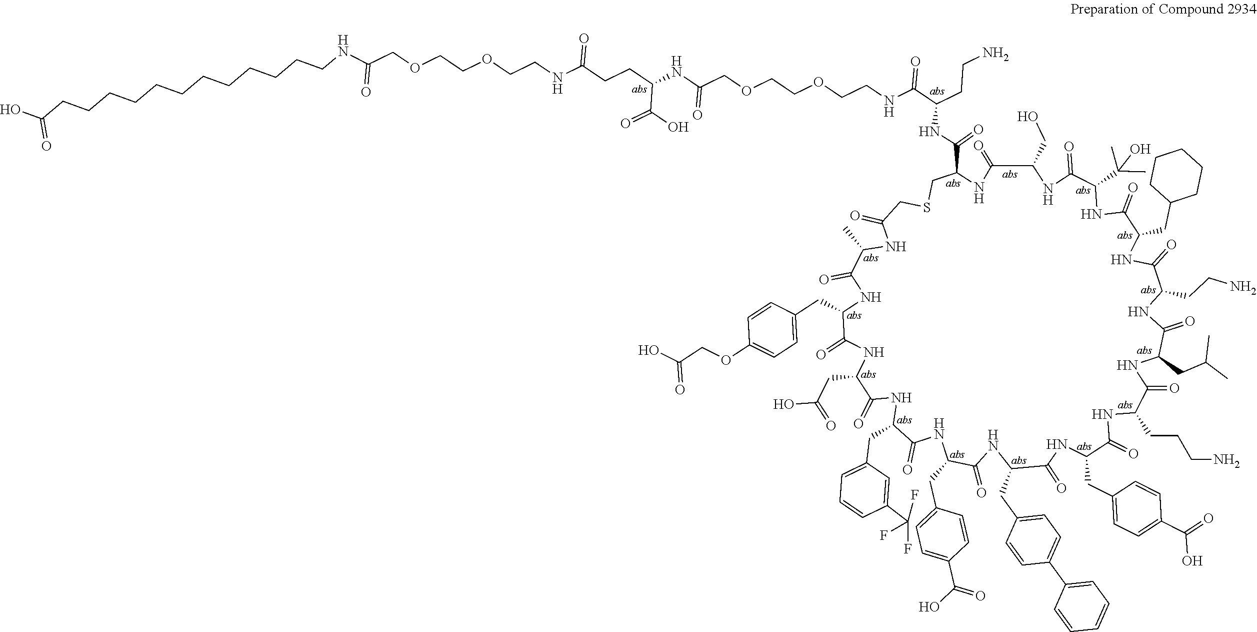 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Compound 2934 was prepared on a 50 μmol scale. The yield of the product was 12.8 mg, and its estimated purity by LCMS analysis was 95%. Analysis condition A: Retention time-1.61 min; ESI-MS(+) m/z [M+2H]²⁺: 1402.1.

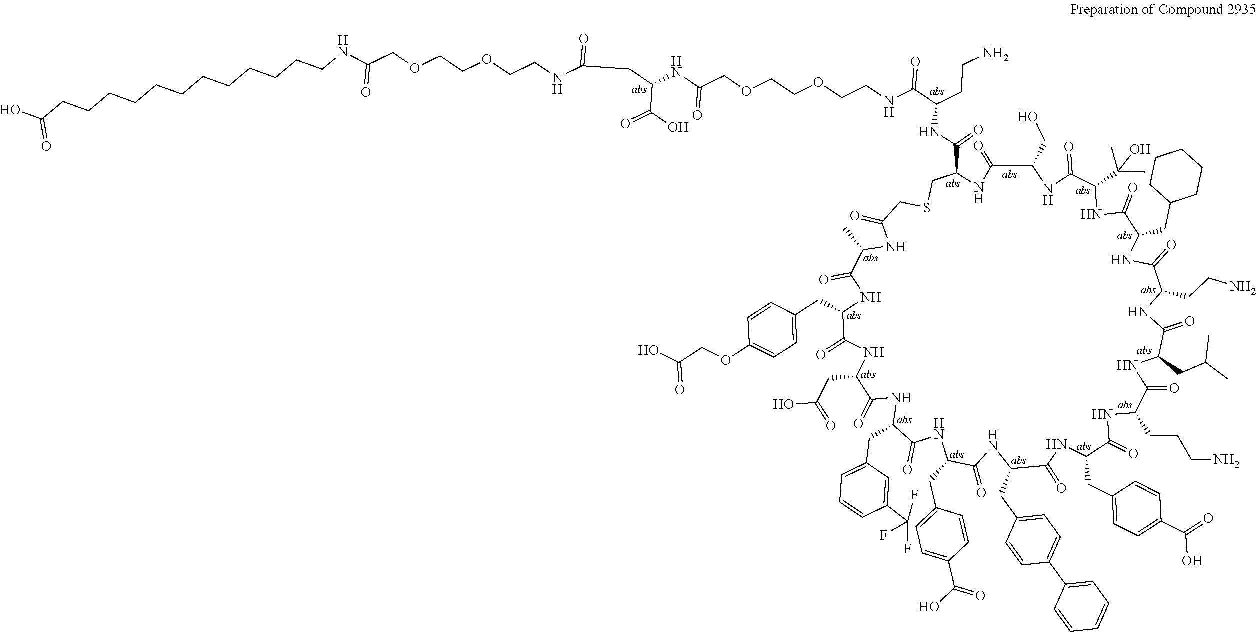 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCOCCOCC(=O)N[C@@H](CC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Compound 2935 was prepared on a 50 μmol scale. The yield of the product was 8.8 mg, and its estimated purity by LCMS analysis was 100%. Analysis condition A: Retention time=1.61 min; ESI-MS(+) m/z [M+2H]²⁺: 1394.9.

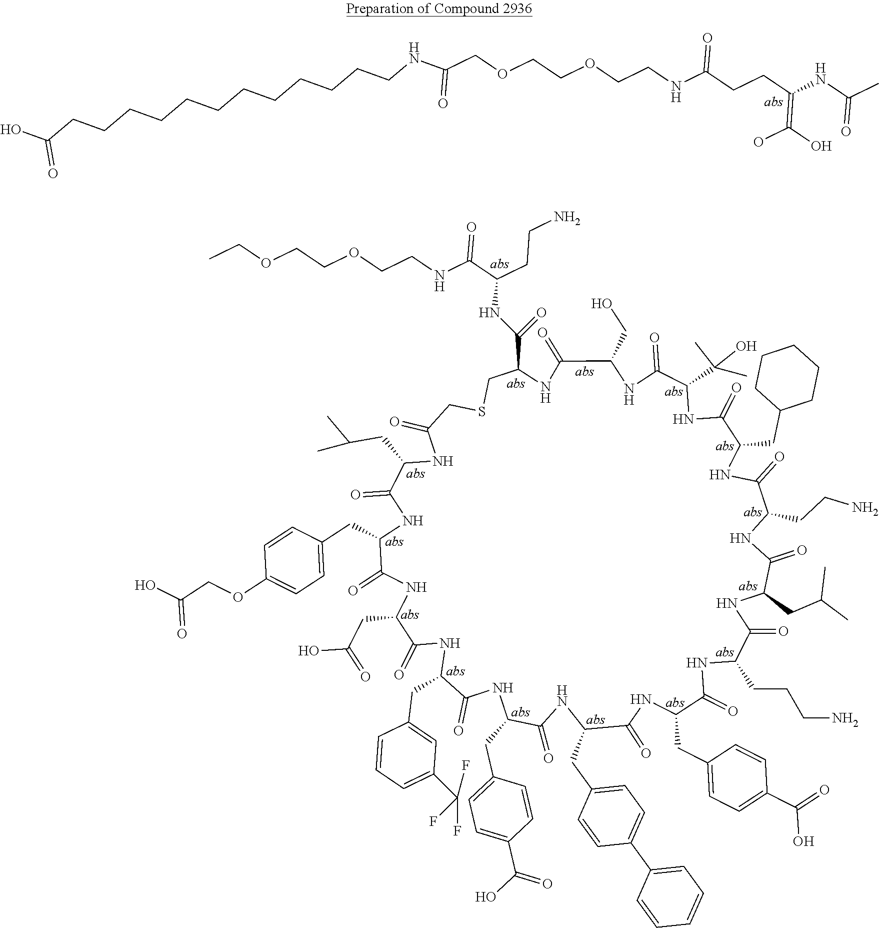

Compound 2936 was prepared on a 50 μmol scale. The yield of the product was 16.3 mg, and its estimated purity by LCMS analysis was 100%. Analysis condition A: Retention time=1.64 min; ESI-MS(+) m/z [M+2H]²⁺: 1423.1.

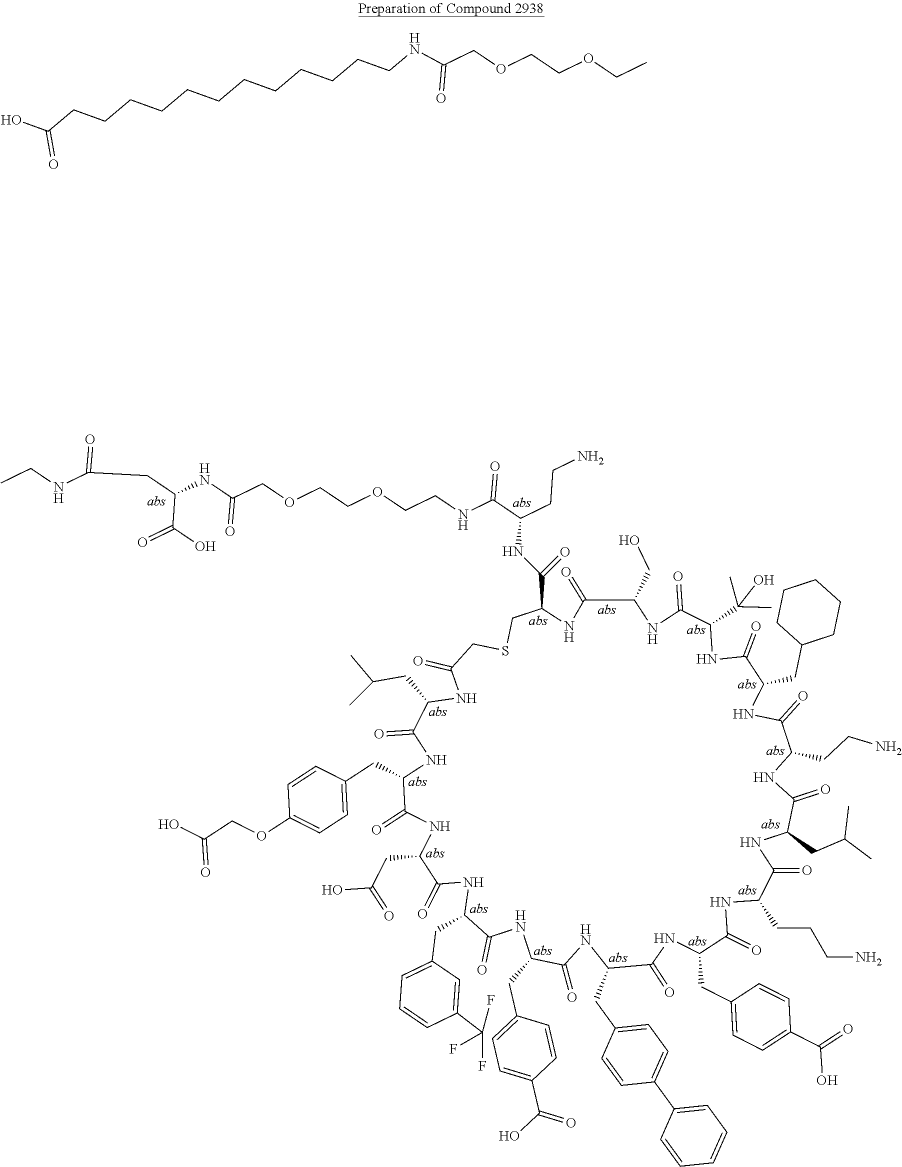

Compound 2937 was prepared on a 50 μmol scale. The yield of the product was 10.1 mg, and its estimated purity by LCMS analysis was 100%. Analysis condition A: Retention time=1.64 min; ESI-MS(+) m/z [M+2H]²⁺: 1416.1.

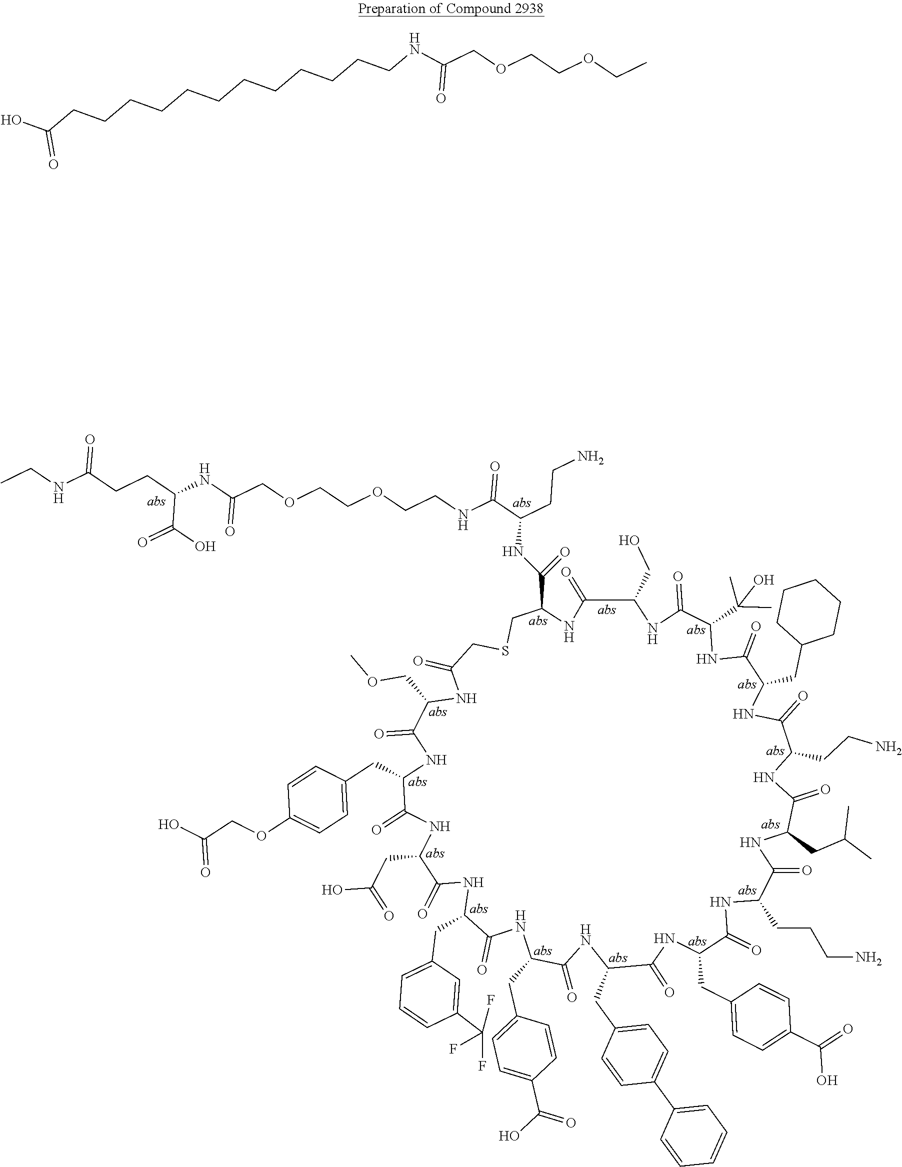

Compound 2938 was prepared on a 50 μmol scale. The yield of the product was 18.5 mg, and its estimated purity by LCMS analysis was 100%. Analysis condition A: Retention time=1.6 min; ESI-MS(+) m/z [M+2H]²⁺: 1417.2.

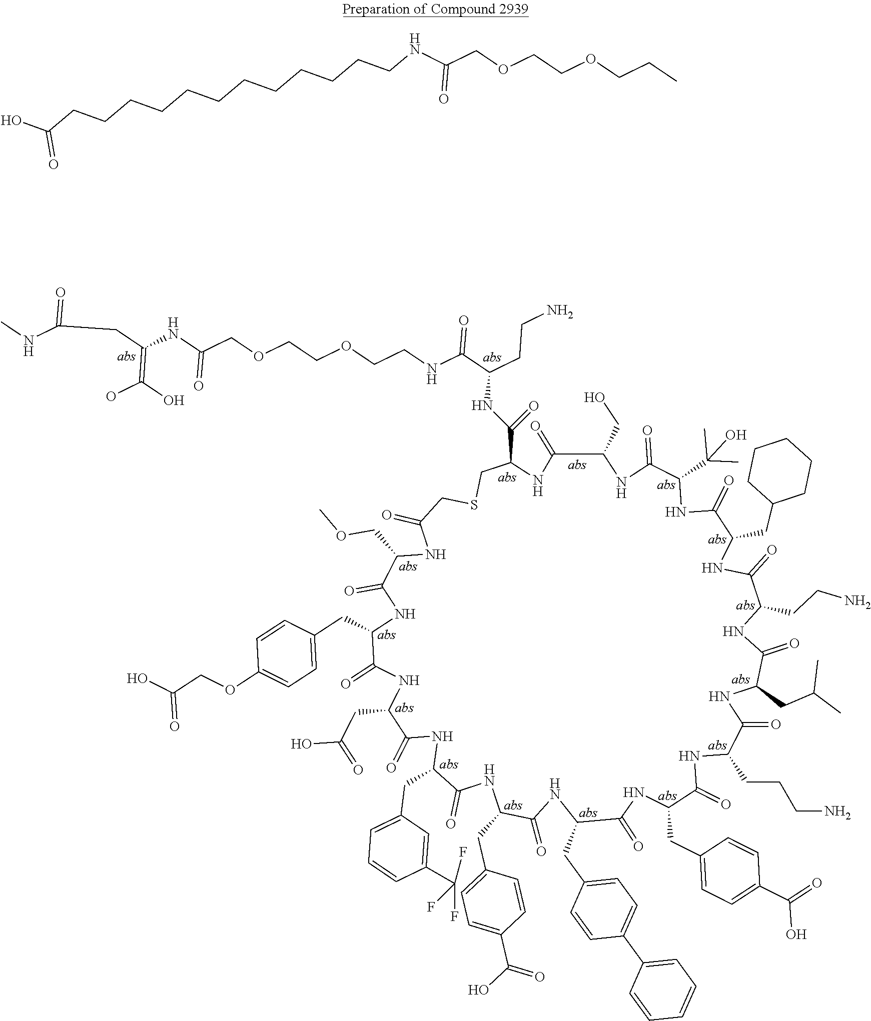

Compound 2939 was prepared on a 50 μmol scale. The yield of the product was 17.6 mg, and its estimated purity by LCMS analysis was 91.5%. Analysis condition A: Retention time=1.67 min; ESI-MS(+) m/z [M+2H]²⁺: 1410.

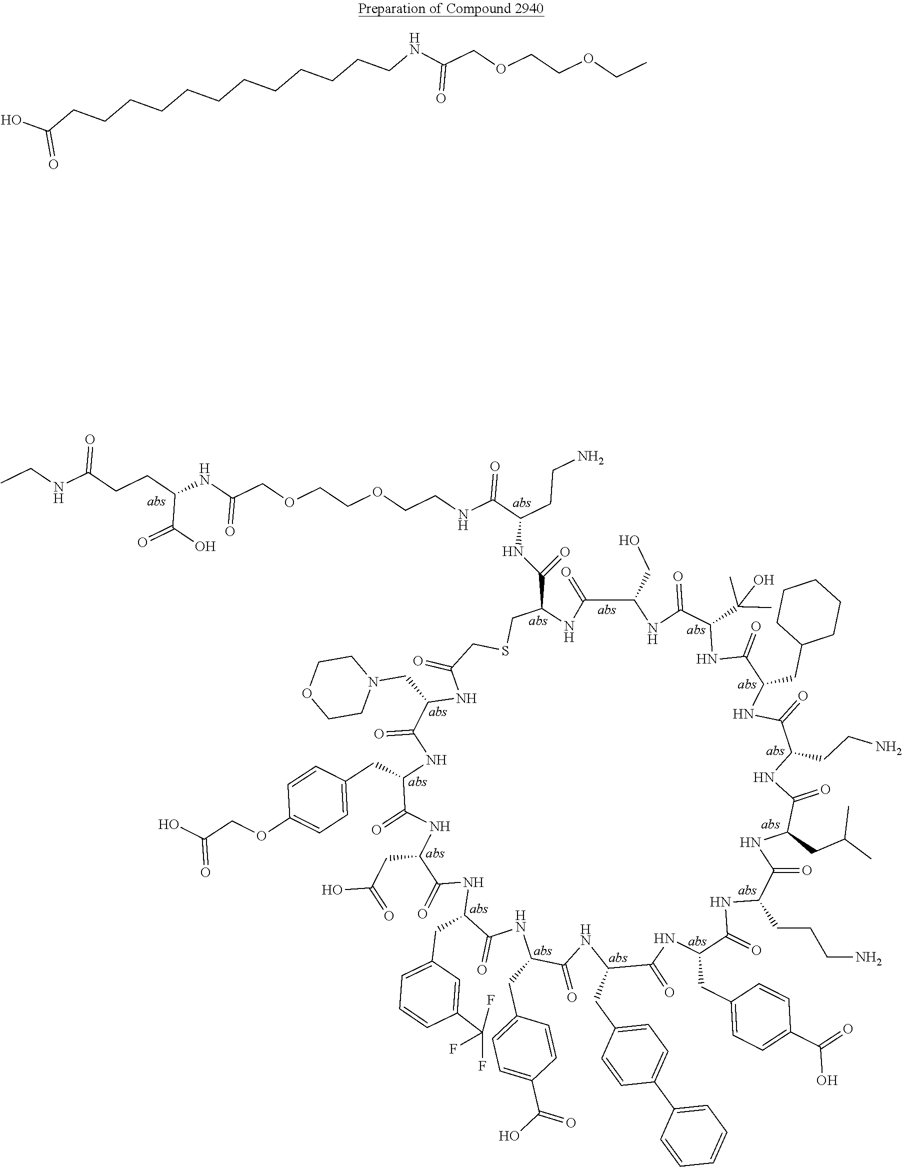

Compound 2940 was prepared on a 50 μmol scale. The yield of the product was 12.3 mg, and its estimated purity by LCMS analysis was 96.1%. Analysis condition B: Retention time=1.81 min; ESI-MS(+) m/z [M+2H]²⁺: 963.5.

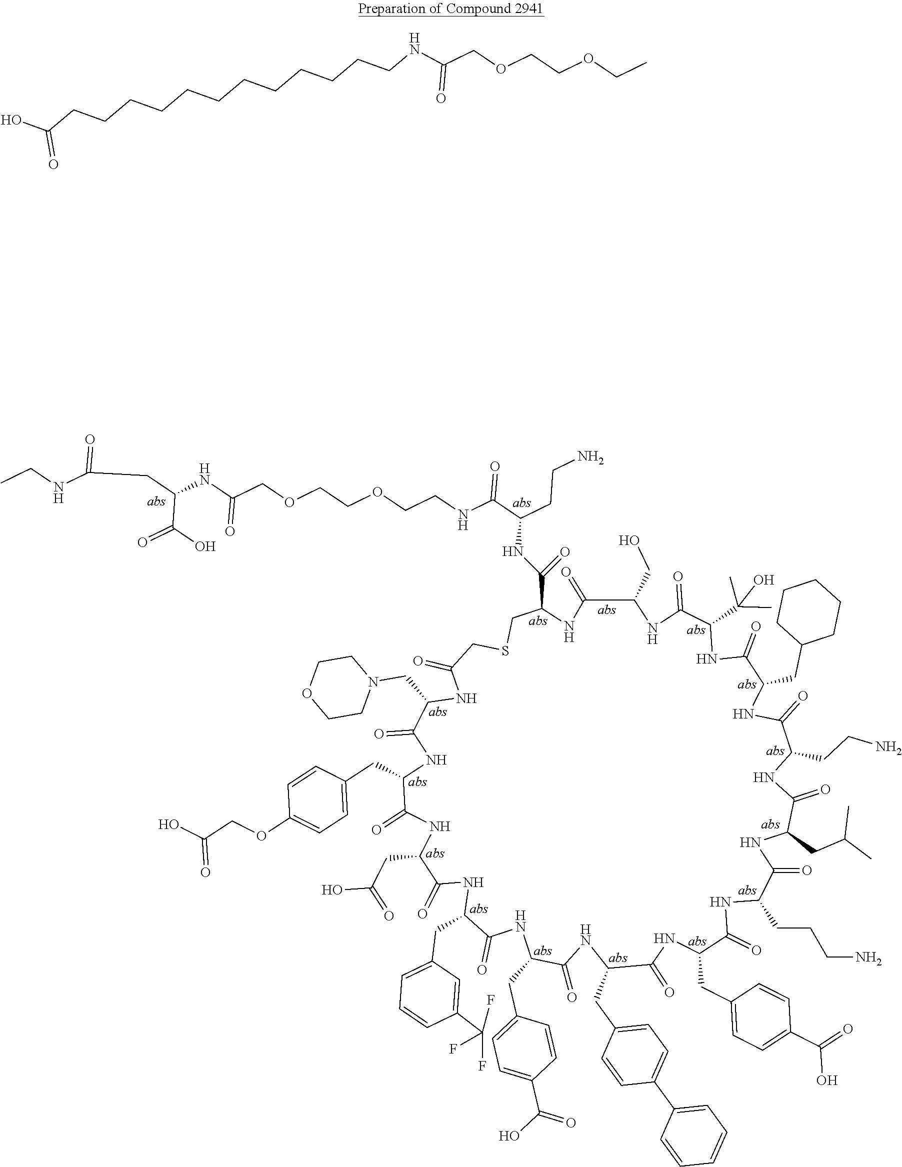

Compound 2941 was prepared on a 50 μmol scale. The yield of the product was 6.7 mg, and its estimated purity by LCMS analysis was 97.6%. Analysis condition A: Retention time-1.57 min; ESI-MS(+) m/z [M+2H]²⁺: 1438.

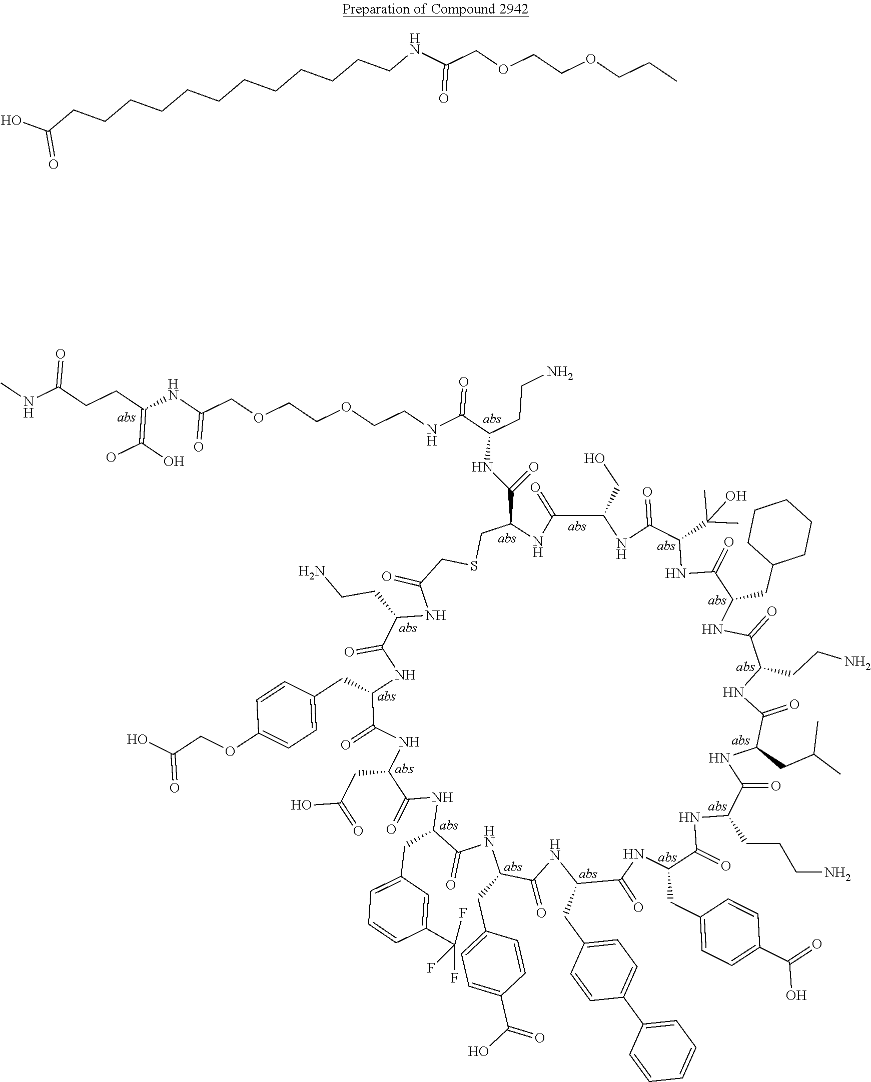

Compound 2942 was prepared on a 50 μmol scale. The yield of the product was 12 mg, and its estimated purity by LCMS analysis was 90.1%. Analysis condition B: Retention time=1.84 min; ESI-MS(+) m/z [M+3H]³⁺: 945.1.

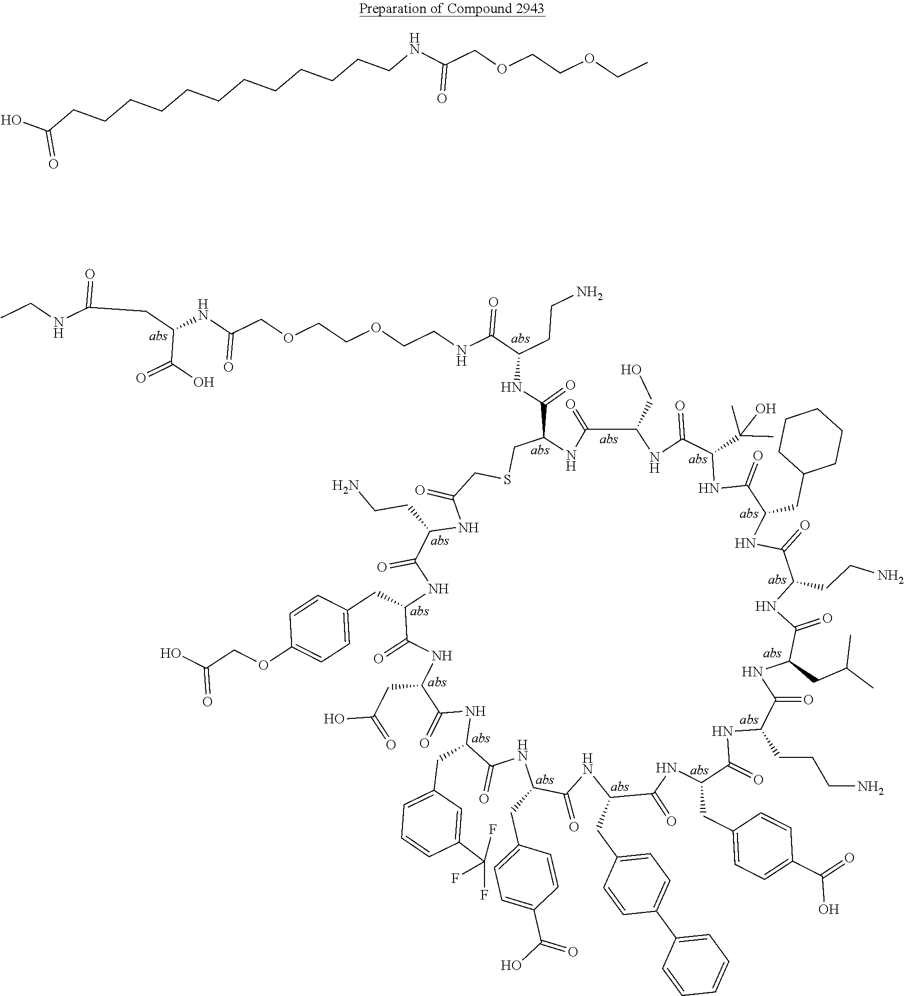

Compound 2943 was prepared on a 50 μmol scale. The yield of the product was 8.5 mg, and its estimated purity by LCMS analysis was 96.6%. Analysis condition A: Retention time=1.67 min; ESI-MS(+) m/z [M+3H]³⁺: 940.3.

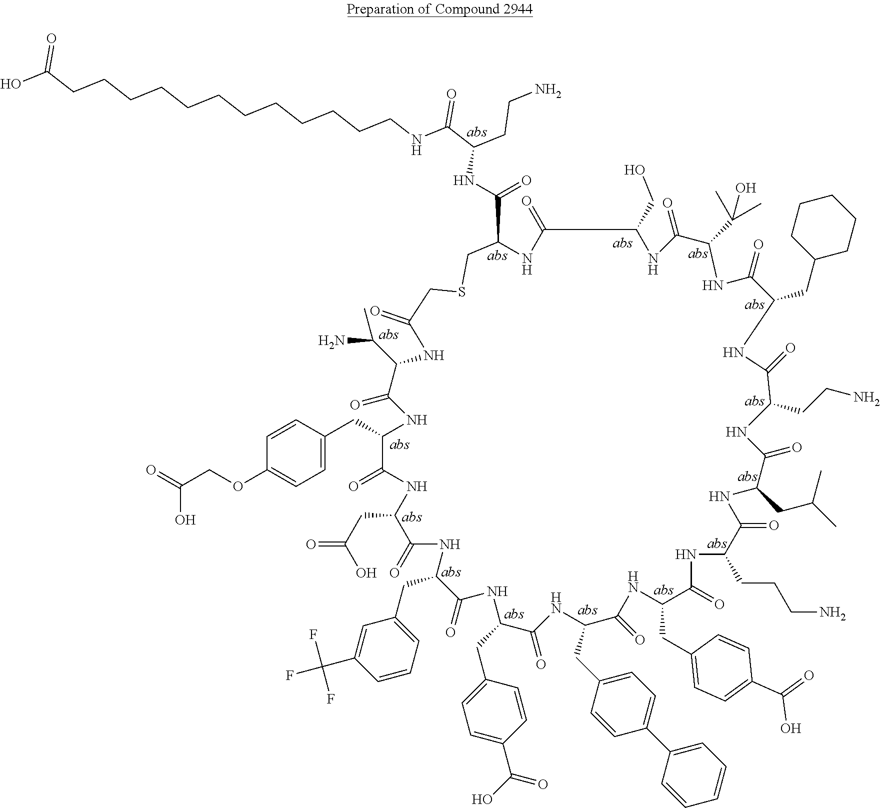 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)N)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 2944 was prepared on a 50 μmol scale. The yield of the product was 1 mg, and its estimated purity by LCMS analysis was 94.8%. Analysis condition B: Retention time=1.78 min; ESI-MS(+) m/z [M+2H]²⁺: 1207.

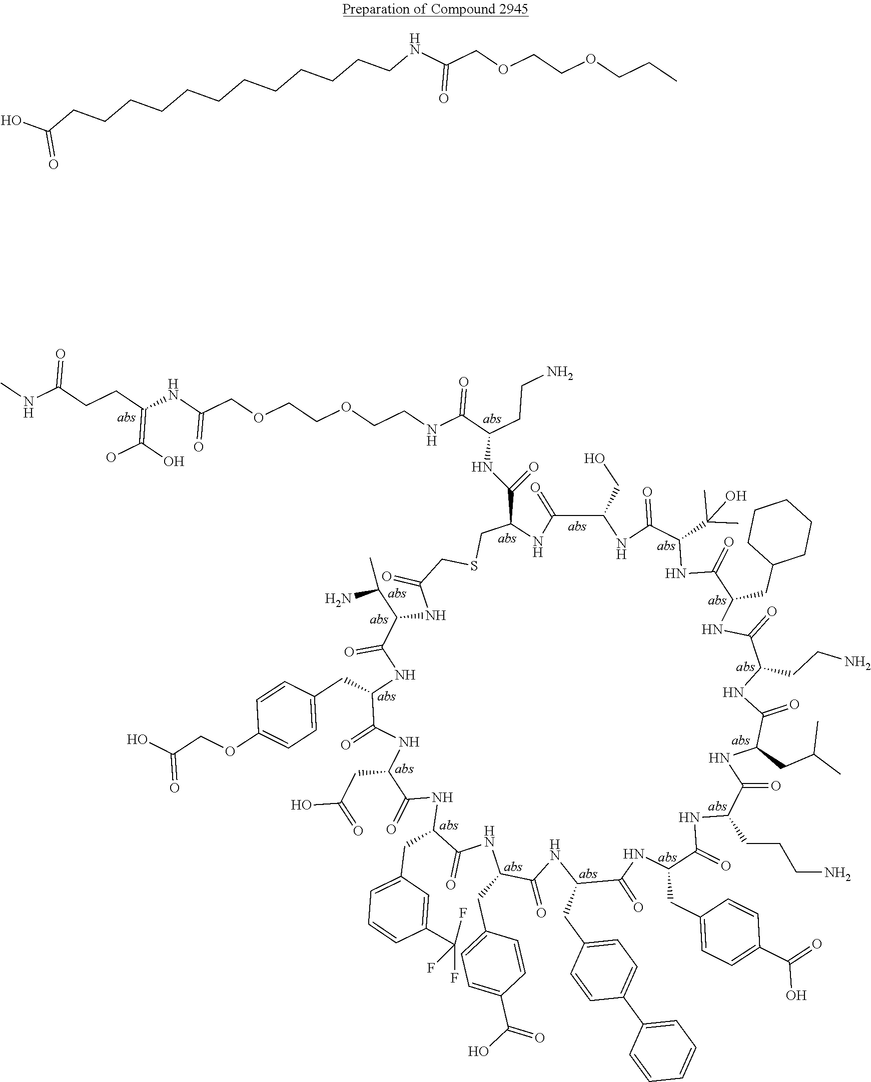

Compound 2945 was prepared on a 50 μmol scale. The yield of the product was 4.2 mg, and its estimated purity by LCMS analysis was 95%. Analysis condition A: Retention time=1.6 min; ESI-MS(+) m/z [M+2H]²⁺: 1416.2.

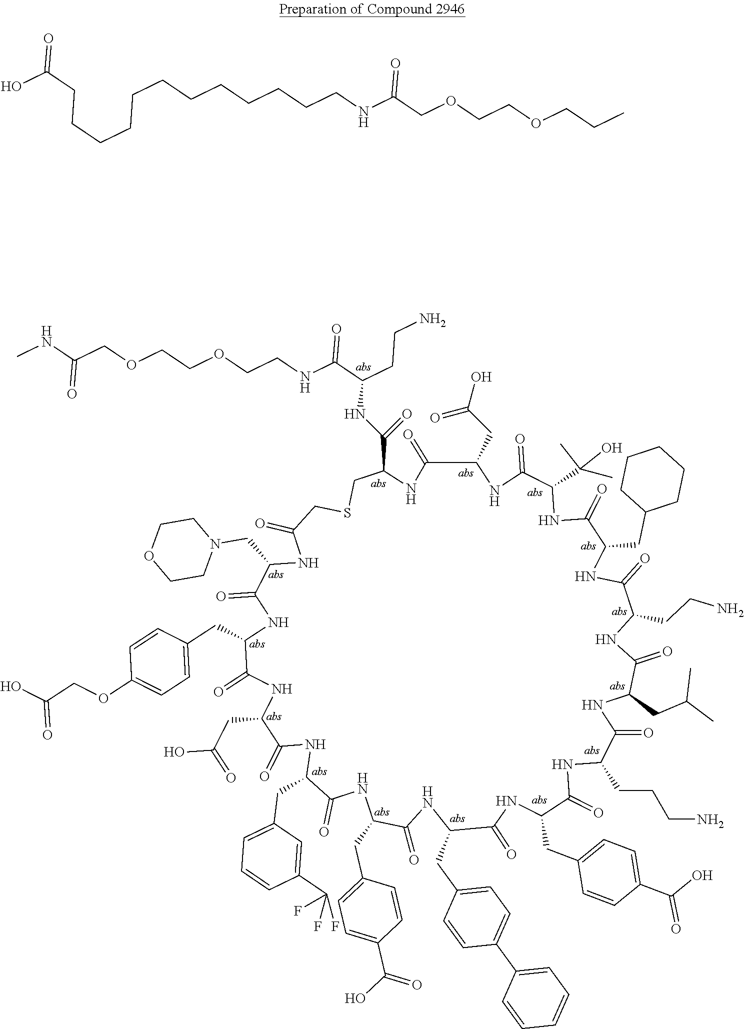 [SMILES: CCCOCCOCC(=O)NCCCCCCCCCCCCC(=O)O ; CNC(=O)COCCOCCNC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C(F)(F)F)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CC(=O)O)C(=O)N1]

Compound 2946 was prepared on a 50 μmol scale. The yield of the product was 17 mg, and its estimated purity by LCMS analysis was 96.1%. Analysis condition B: Retention time=1.82 min; ESI-MS(+) m/z [M+2H]²⁺: 1408.1.

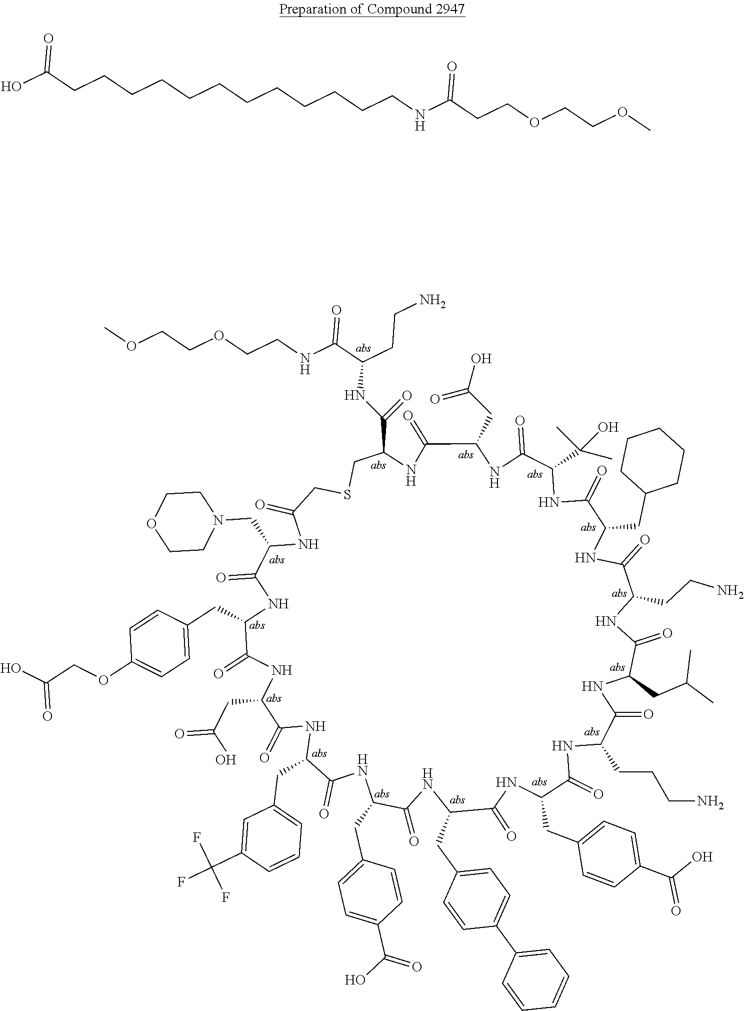 [SMILES: COCCOCCC(=O)NCCCCCCCCCCCCC(=O)O ; COCCOCCNC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C(F)(F)F)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CC(=O)O)C(=O)N1]

Compound 2947 was prepared on a 50 μmol scale. The yield of the product was 26.7 mg, and its estimated purity by LCMS analysis was 92.6%. Analysis condition B: Retention time=1.84 min; ESI-MS(+) m/z [M+2H]²⁺: 1373.

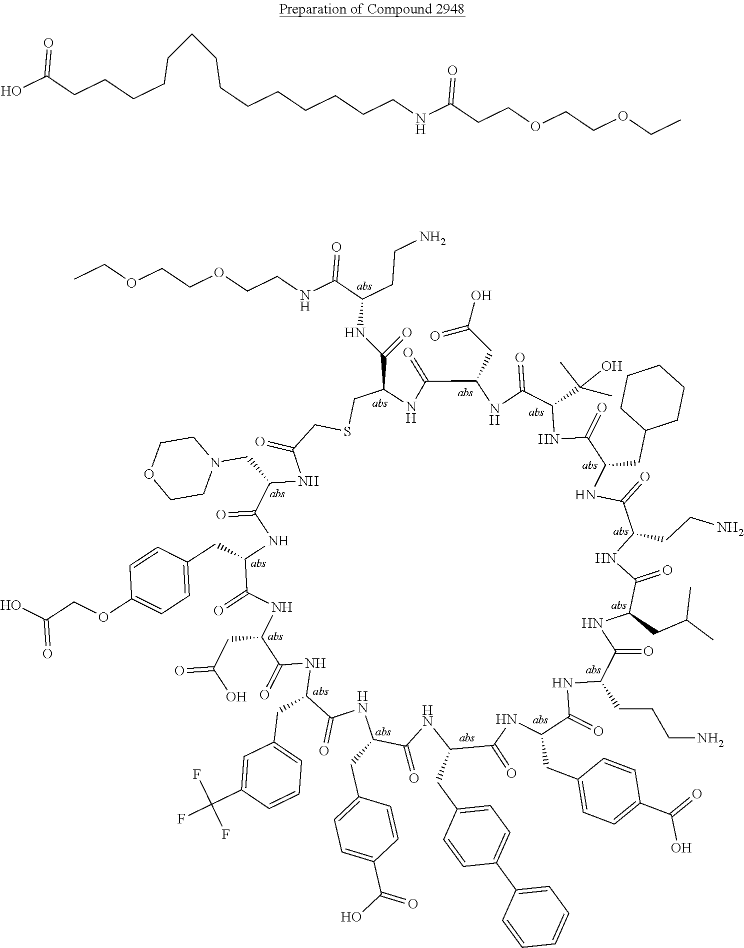 [SMILES: CCOCCOCCC(=O)NCCCCCCCCCCCCCCC(=O)O ; CCOCCOCCNC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C(F)(F)F)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CC(=O)O)C(=O)N1]

Compound 2948 was prepared on a 50 μmol scale. The yield of the product was 11.4 mg, and its estimated purity by LCMS analysis was 98.3%. Analysis condition B: Retention time=1.91 min; ESI-MS(+) m/z [M+3H]³⁺: 924.4.

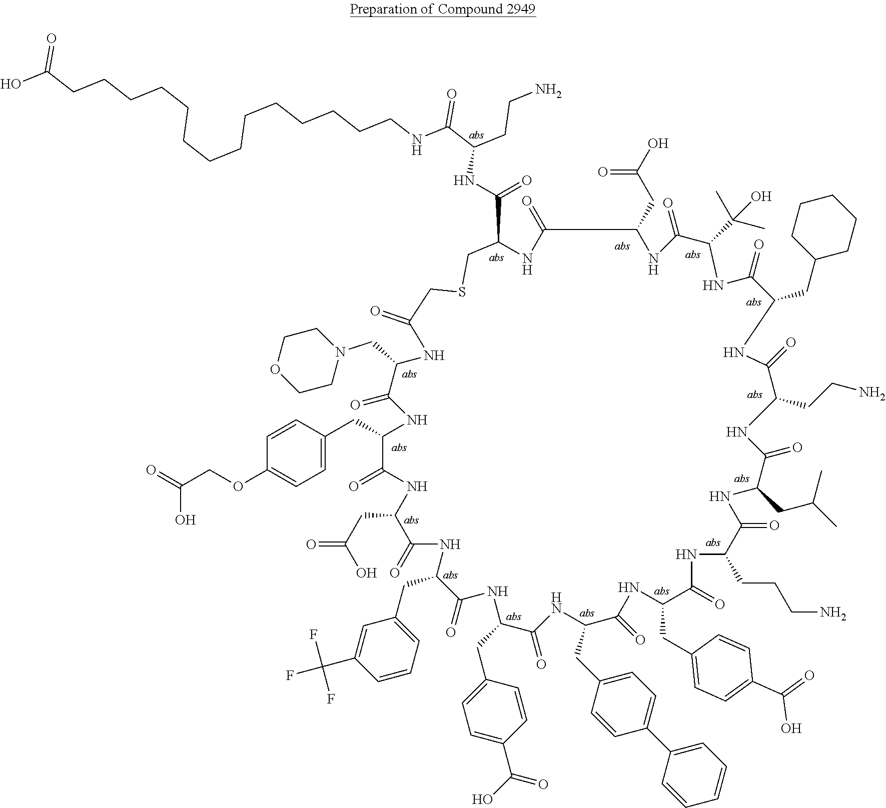 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(=O)NCCCCCCCCCCCCCCC(=O)O)NC(=O)[C@H](CC(=O)O)NC(=O)[C@H](C(C)(C)O)NC(=O)[C@H](CC2CCCCC2)NC(=O)[C@H](CCN)NC1=O]

Compound 2949 was prepared on a 50 μmol scale. The yield of the product was 7.7 mg, and its estimated purity by LCMS analysis was 95.7%. Analysis condition A: Retention time=1.55 min; ESI-MS(+) m/z [M+2H]²⁺: 1263.1.

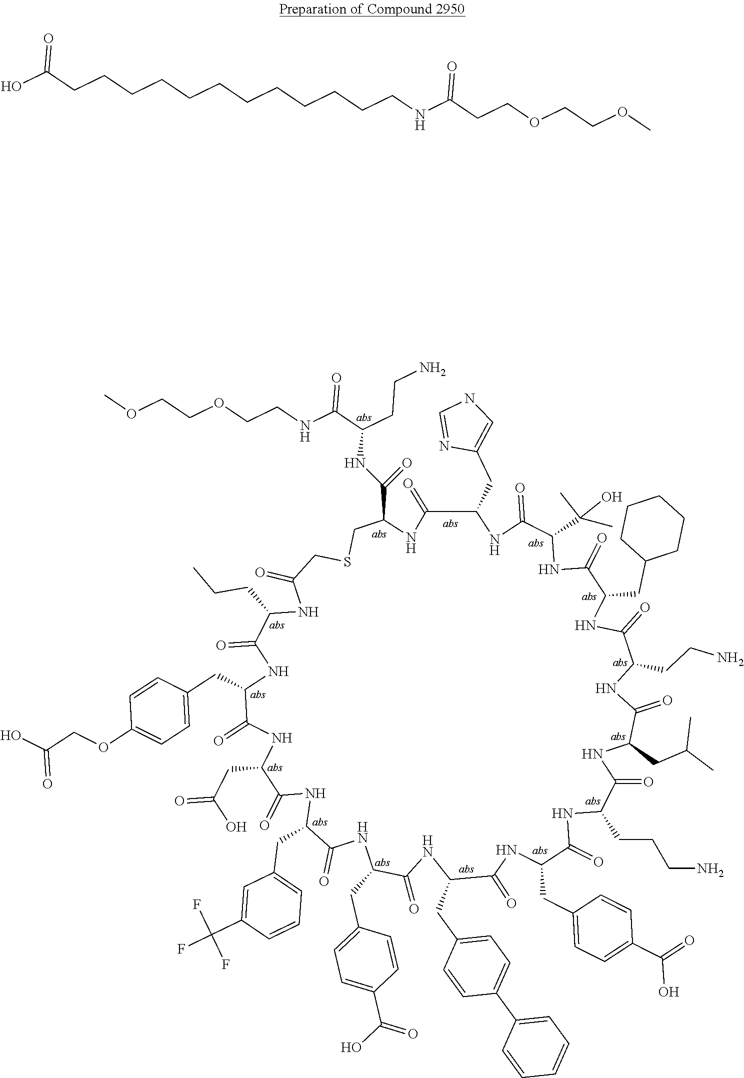 [SMILES: COCCOCCC(=O)NCCCCCCCCCCCCC(=O)O ; CCC[C@@H]1NC(=O)CSC[C@@H](C(=O)N[C@@H](CCN)C(=O)NCCOCCOC)NC(=O)[C@H](Cc2c[nH]cn2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2950 was prepared on a 50 μmol scale. The yield of the product was 12.8 mg, and its estimated purity by LCMS analysis was 95.5%. Analysis condition B: Retention time=1.84 min; ESI-MS(+) m/z [M+2H]²⁺: 1355.2.

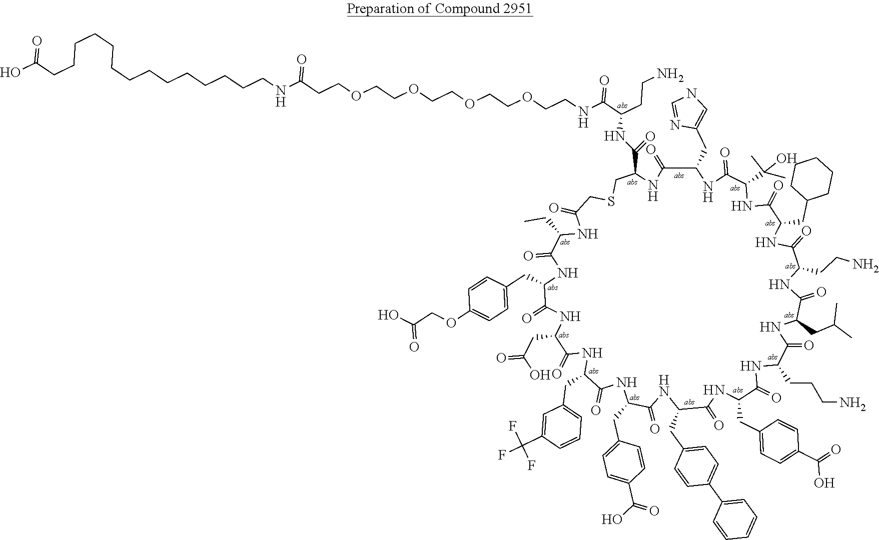 [SMILES: CC[C@@H]1NC(=O)CSC[C@@H](C(=O)N[C@@H](CCN)C(=O)NCCOCCOCCOCCOCCC(=O)NCCCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 2951 was prepared on a 50 μmol scale. The yield of the product was 6.8 mg, and its estimated purity by LCMS analysis was 95.4%. Analysis condition A: Retention time=1.71 min; ESI-MS(+) m/z [M+2H]²⁺: 1368.4.

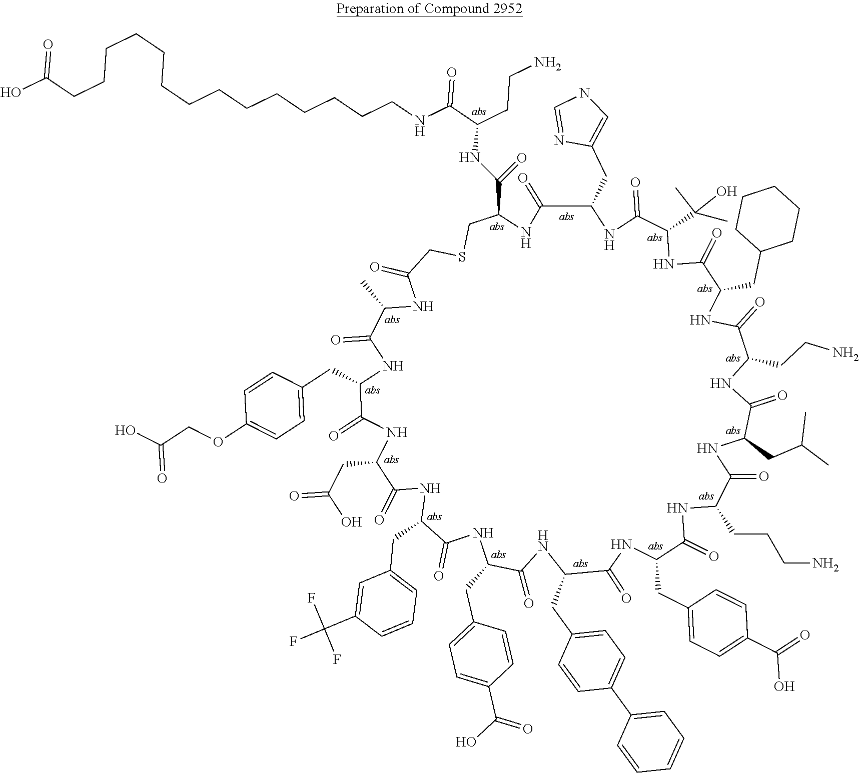 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Compound 2952 was prepared on a 50 μmol scale. The yield of the product was 44.3 mg, and its estimated purity by LCMS analysis was 94.8%. Analysis condition A: Retention time=1.72 min; ESI-MS(+) m/z [M+3H]³⁺: 831.1.

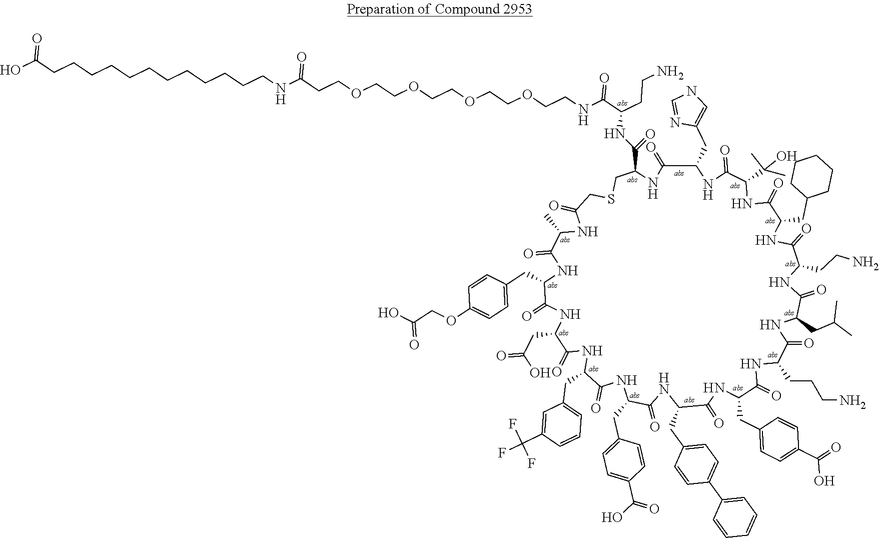 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCOCCOCCOCCOCCC(=O)NCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Compound 2953 was prepared on a 50 μmol scale. The yield of the product was 9.5 mg, and its estimated purity by LCMS analysis was 99.4%. Analysis condition A: Retention time=1.58 min; ESI-MS(+) m/z [M+2H]²⁺: 1341.1.

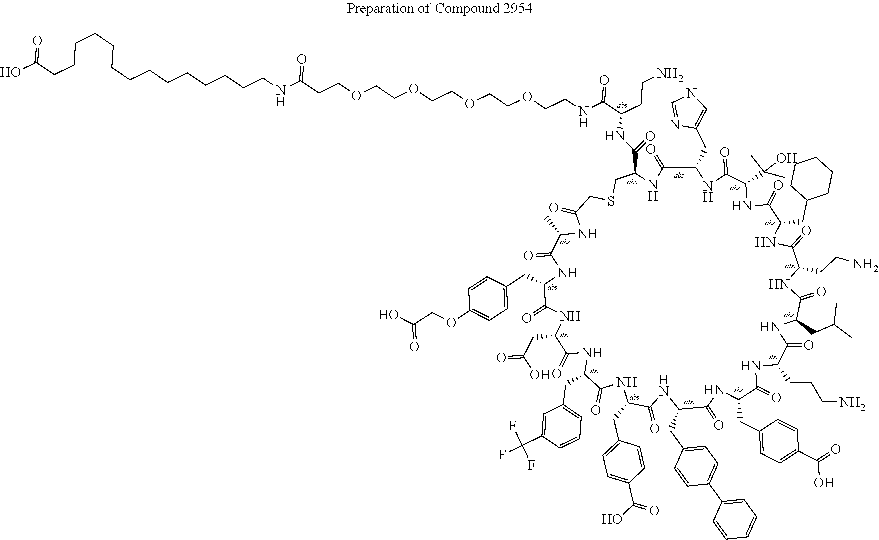 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCOCCOCCOCCOCCC(=O)NCCCCCCCCCCCCCCC(=O)O)NC(=O)[C@H](Cc2c[nH]cn2)NC(=O)[C@H](C(C)(C)O)NC(=O)[C@H](CC2CCCCC2)NC(=O)[C@H](CCN)NC1=O]

Compound 2954 was prepared on a 50 μmol scale. The yield of the product was 5.8 mg, and its estimated purity by LCMS analysis was 99.2%. Analysis condition B: Retention time=1.91 min; ESI-MS(+) m/z [M+2H]²⁺: 1355.1.

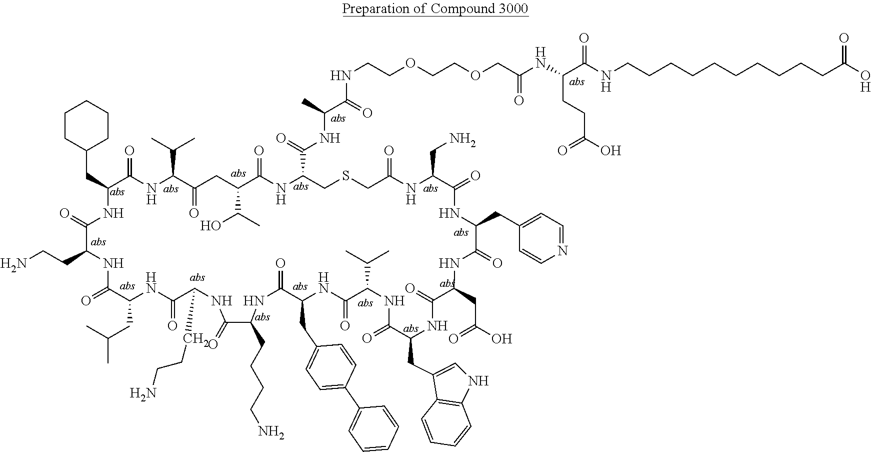 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(=O)NCCOCCOCC(=O)N[C@@H](CCC(=O)O)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CCN)NC1=O]

Compound 3000 was prepared on a 50 μmol scale. The yield of the product was 29.2 mg, and its estimated purity by LCMS analysis was 99%. Analysis condition A: Retention time=1.46 min; ESI-MS(+) m/z [M+3H]³⁺: 786.7.

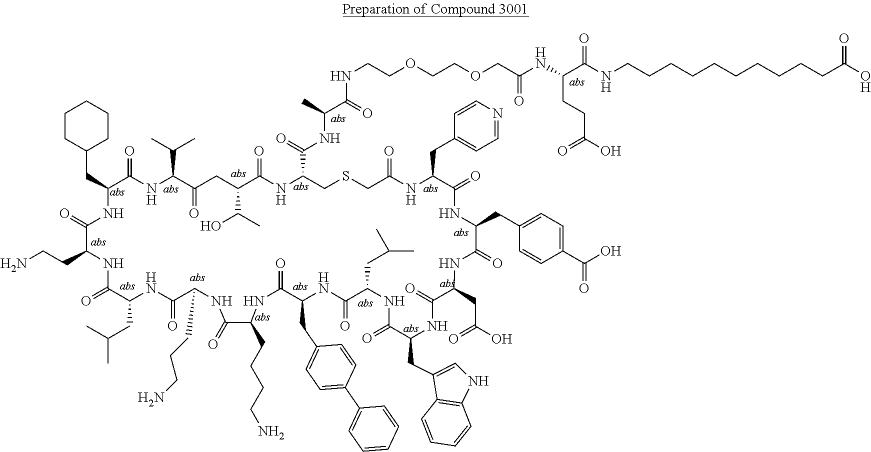 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(=O)NCCOCCOCC(=O)N[C@@H](CCC(=O)O)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCCN)NC(=O)[C@H](Cc2ccc(-c3ccccc3)cc2)NC1=O]

Compound 3001 was prepared on a 50 μmol scale. The yield of the product was 50.5 mg, and its estimated purity by LCMS analysis was 95.7%. Analysis condition A: Retention time=1.51 min; ESI-MS(+) m/z [M+3H]³⁺: 826.5.

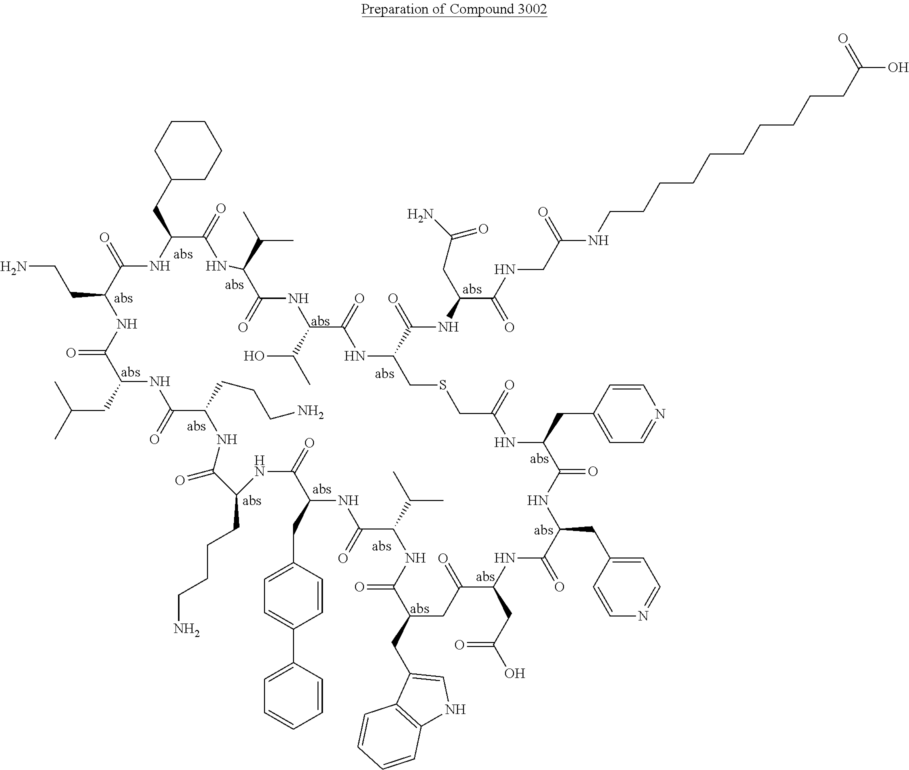 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)CC(=O)[C@H](CC(=O)O)NC(=O)[C@H](Cc2ccncc2)NC(=O)[C@H](Cc2ccncc2)NC(=O)CSC[C@@H](C(=O)N[C@@H](CC(N)=O)C(=O)NCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3002 was prepared on a 50 μmol scale. The yield of the product was 30.1 mg, and its estimated purity by LCMS analysis was 98%. Analysis condition B: Retention time=1.28 min; ESI-MS(+) m/z [M+3H]³⁺: 749.2.

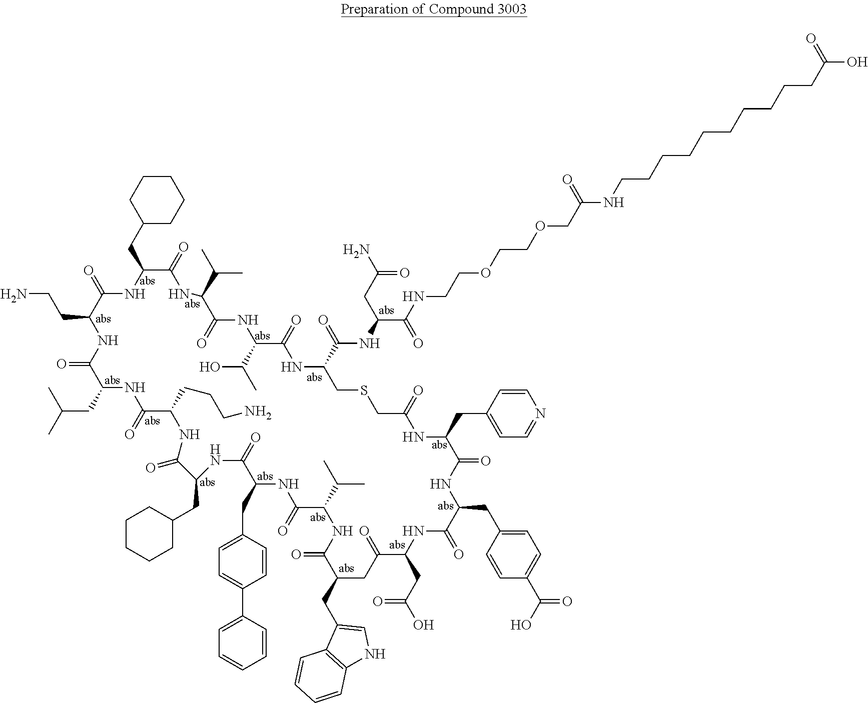 [SMILES: CC(C)C[C@H]1NC(=O)[C@H](CCCN)NC(=O)[C@H](CC2CCCCC2)NC(=O)[C@H](Cc2ccc(-c3ccccc3)cc2)NC(=O)[C@H](C(C)C)NC(=O)[C@H](Cc2c[nH]c3ccccc23)CC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CC(N)=O)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3003 was prepared on a 50 μmol scale. The yield of the product was 58.4 mg, and its estimated purity by LCMS analysis was 94.7%. Analysis condition B: Retention time=1.85 min; ESI-MS(+) m/z [M+3H]³⁺: 801.3.

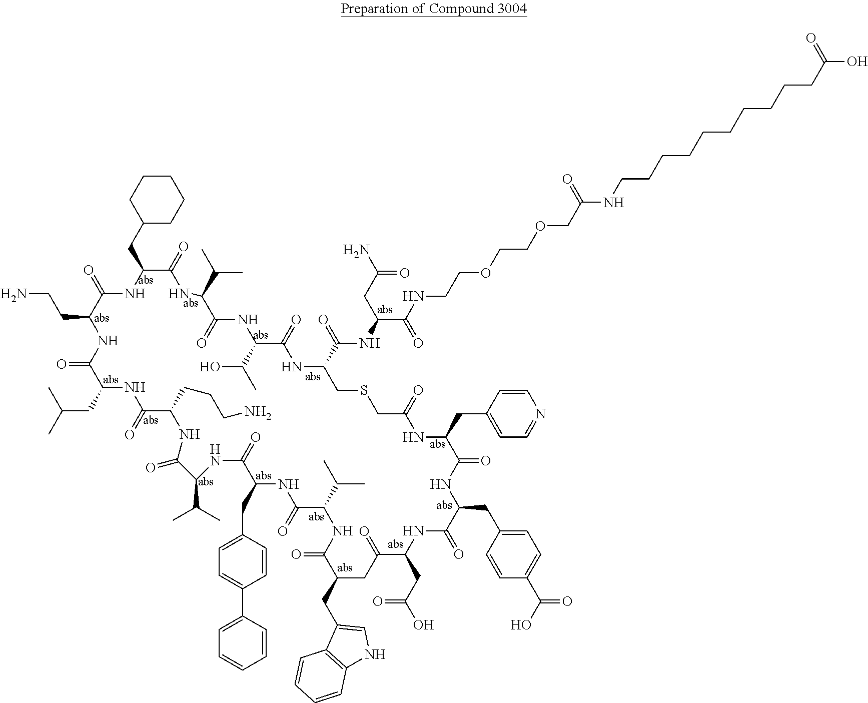 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)CC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CC(N)=O)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3004 was prepared on a 50 μmol scale. The yield of the product was 22.1 mg, and its estimated purity by LCMS analysis was 95.8%. Analysis condition B: Retention time=1.71, 1.75 min; ESI-MS(+) m/z [M+3H]³⁺: 783.22, 1174.1.

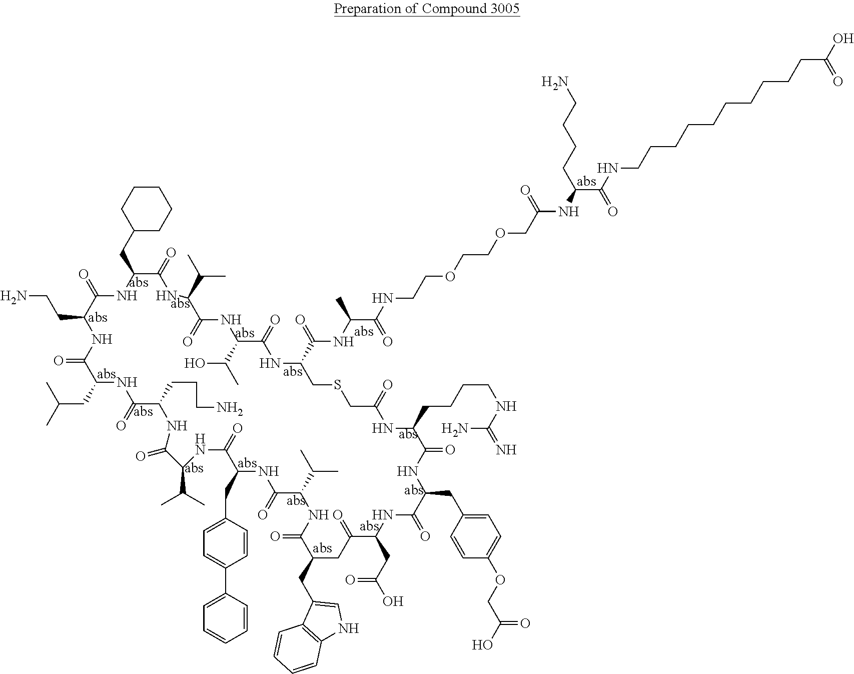 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)CC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](C)C(=O)NCCOCCOCC(=O)N[C@@H](CCCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3005 was prepared on a 50 μmol scale. The yield of the product was 21.1 mg, and its estimated purity by LCMS analysis was 96.8%. Analysis condition A: Retention time=1.55 min; ESI-MS(+) m/z [M+2H]²⁺: 1243.1.

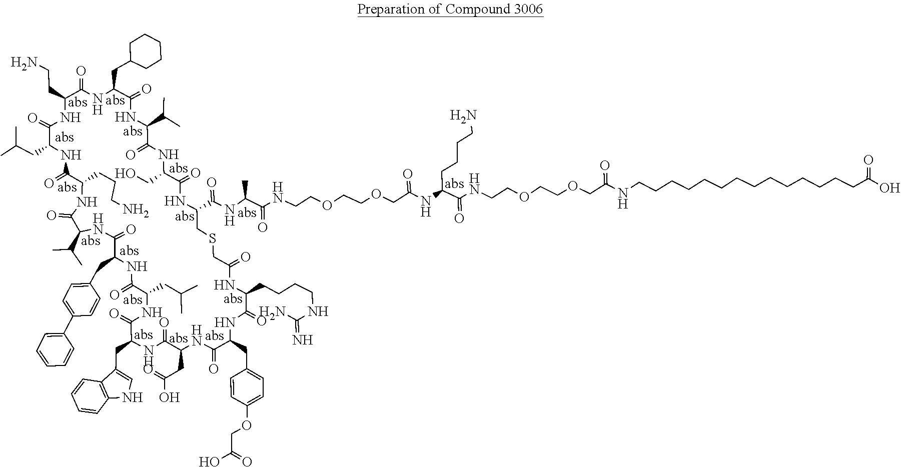

Compound 3006 was prepared on a 50 μmol scale. The yield of the product was 18.9 mg, and its estimated purity by LCMS analysis was 100%. Analysis condition A: Retention time=1.85 min; ESI-MS(+) m/z [M+3H]³⁺: 896.2.

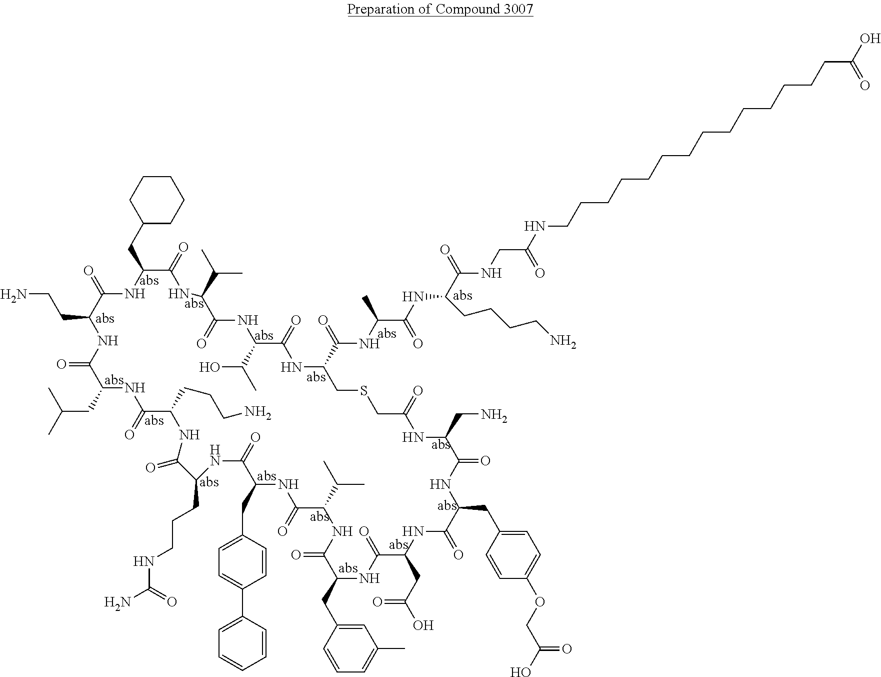 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCCCN)C(=O)NCC(=O)NCCCCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3007 was prepared on a 50 μmol scale. The yield of the product was 36.5 mg, and its estimated purity by LCMS analysis was 98.6%. Analysis condition A: Retention time=1.75 min; ESI-MS(+) m/z [M+2H]²⁺: 1201.1.

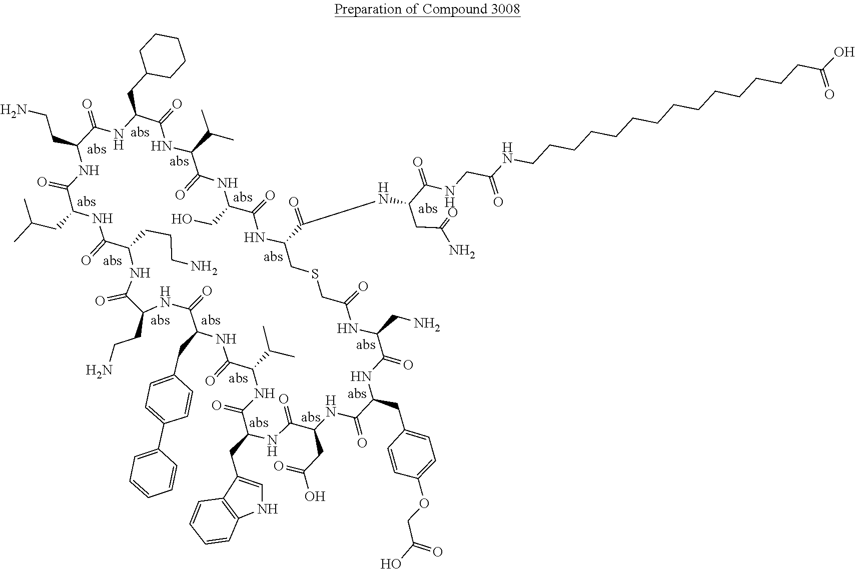 [SMILES: CC(C)C[C@H]1NC(=O)[C@H](CCCN)NC(=O)[C@H](CCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CC(N)=O)C(=O)NCC(=O)NCCCCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3008 was prepared on a 50 μmol scale. The yield of the product was 14 mg, and its estimated purity by LCMS analysis was 95.1%. Analysis condition A: Retention time=1.84 min; ESI-MS(+) m/z [M+2H]²⁺: 1136.

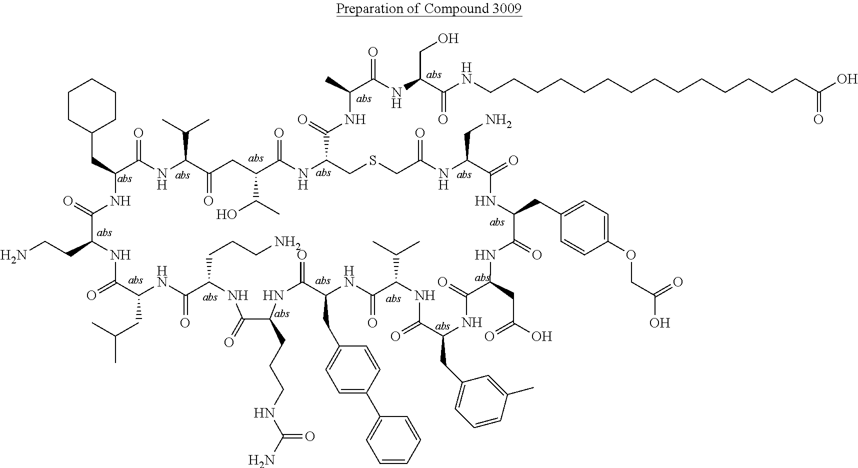 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CO)C(=O)NCCCCCCCCCCCCCCC(=O)O)NC(=O)[C@H](C(C)O)CC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3009 was prepared on a 50 μmol scale. The yield of the product was 17.6 mg, and its estimated purity by LCMS analysis was 96.2%. Analysis condition A: Retention time=1.81 min; ESI-MS(+) m/z [M+2H]²⁺: 1152.1.

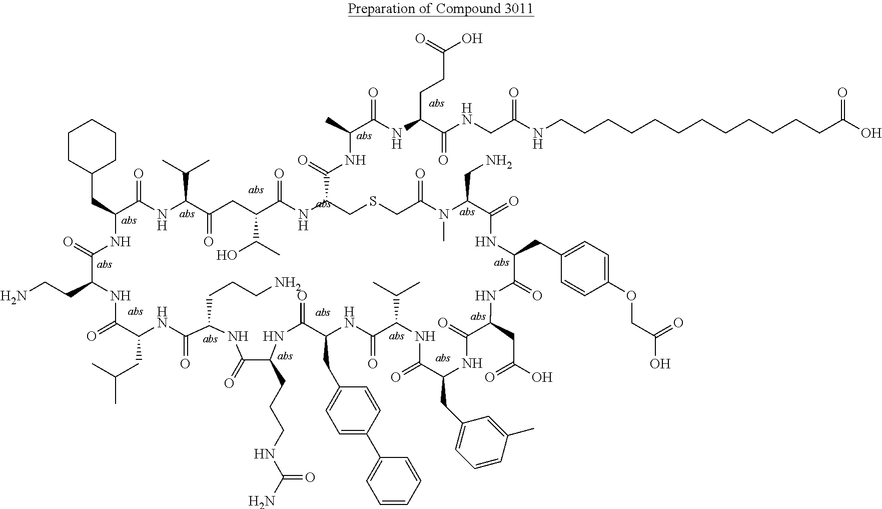 [SMILES: Cc1cccc(C[C@@H]2NC(=O)[C@H](CC(=O)O)NC(=O)[C@H](Cc3ccc(OCC(=O)O)cc3)NC(=O)[C@H](CN)N(C)C(=O)CSC[C@@H](C(=O)N[C@@H](C)C(=O)N[C@@H](CCC(=O)O)C(=O)NCC(=O)NCCCCCCCCCCCCC(=O)O)NC(=O)[C@H](C(C)O)CC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3010 was prepared on a 50 μmol scale. The yield of the product was 26.3 mg, and its estimated purity by LCMS analysis was 97.6%. Analysis condition B: Retention time=1.82 min; ESI-MS(+) m/z [M+2H]²⁺: 1173.1.

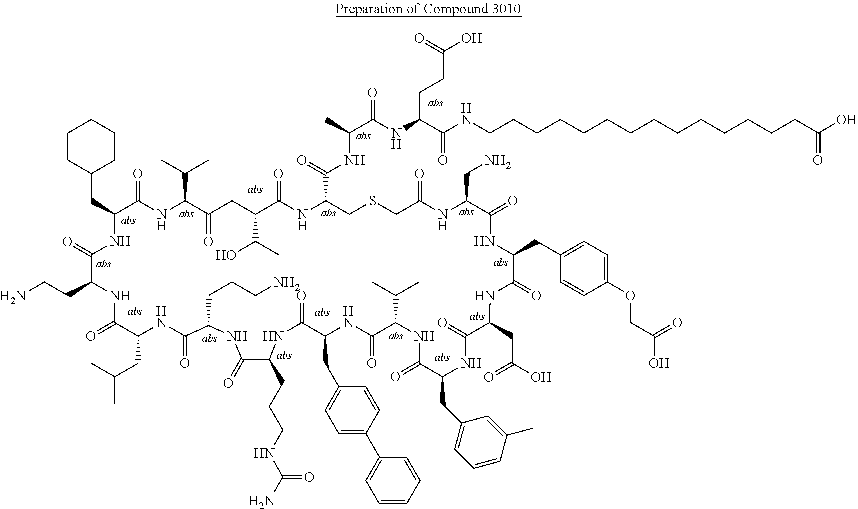 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(=O)NCCCCCCCCCCCCCCC(=O)O)NC(=O)[C@H](C(C)O)CC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3011 was prepared on a 50 μmol scale. The yield of the product was 13.7 mg, and its estimated purity by LCMS analysis was 100%. Analysis condition A: Retention time=1.99 min; ESI-MS(+) m/z [M+2H]²⁺: 1222.

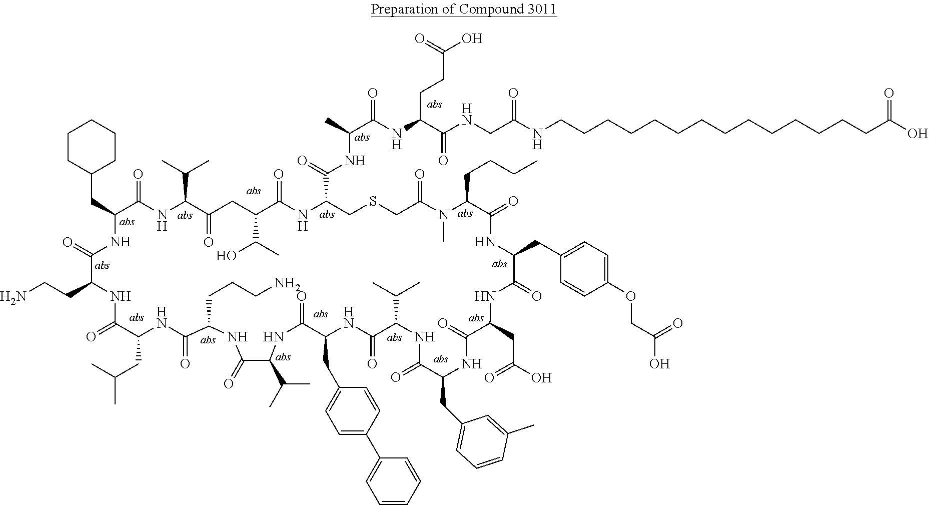 [SMILES: CCCC[C@H]1C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)C[C@@H](C(C)O)C(=O)N[C@H](C(=O)N[C@@H](C)C(=O)N[C@@H](CCC(=O)O)C(=O)NCC(=O)NCCCCCCCCCCCCCCC(=O)O)CSCC(=O)N1C]

Compound 3012 was prepared on a 50 μmol scale. The yield of the product was 23.1 mg, and its estimated purity by LCMS analysis was 100%. Analysis condition A: Retention time=2.23 min; ESI-MS(+) m/z [M+2H]²⁺: 1206.

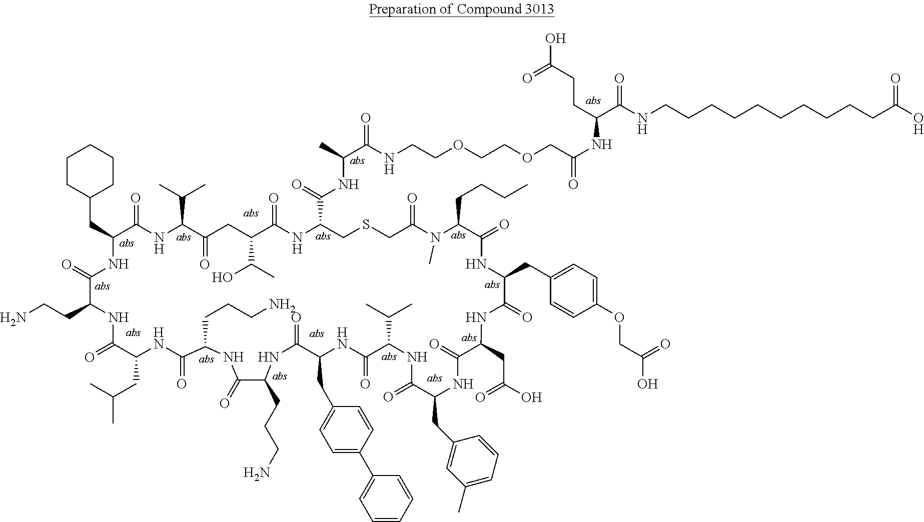 [SMILES: CCCC[C@H]1C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCN)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)C[C@@H](C(C)O)C(=O)N[C@H](C(=O)N[C@@H](C)C(=O)NCCOCCOCC(=O)N[C@@H](CCC(=O)O)C(=O)NCCCCCCCCCCC(=O)O)CSCC(=O)N1C]

Compound 3013 was prepared on a 50 μmol scale. The yield of the product was 44 mg, and its estimated purity by LCMS analysis was 95.2%. Analysis condition A: Retention time=2.04 min; ESI-MS(+) m/z [M+3H]³⁺: 811.4.

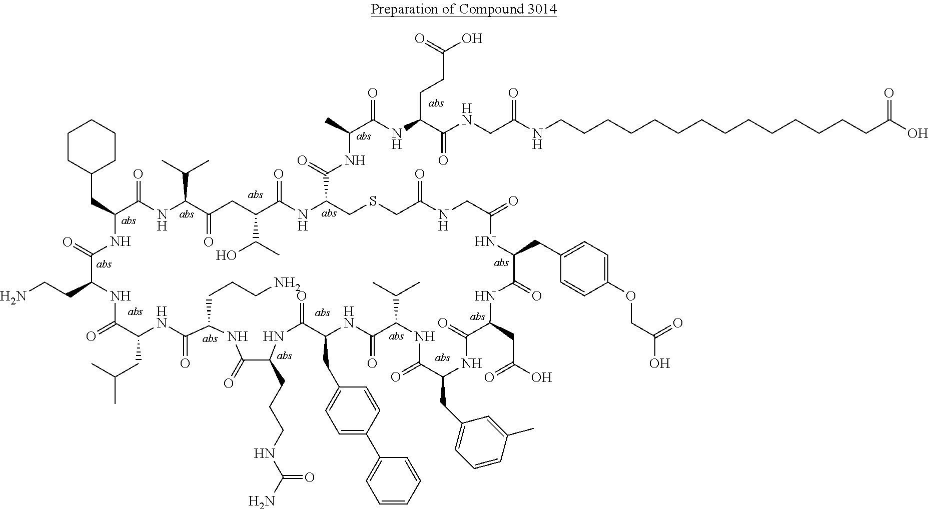 [SMILES: Cc1cccc(C[C@@H]2NC(=O)[C@H](CC(=O)O)NC(=O)[C@H](Cc3ccc(OCC(=O)O)cc3)NC(=O)CNC(=O)CSC[C@@H](C(=O)N[C@@H](C)C(=O)N[C@@H](CCC(=O)O)C(=O)NCC(=O)NCCCCCCCCCCCCCCC(=O)O)NC(=O)[C@H](C(C)O)CC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3014 was prepared on a 50 μmol scale. The yield of the product was 17.2 mg, and its estimated purity by LCMS analysis was 97.3%. Analysis condition B: Retention time=2.14 min; ESI-MS(+) m/z [M+2H]²⁺: 1186.5.

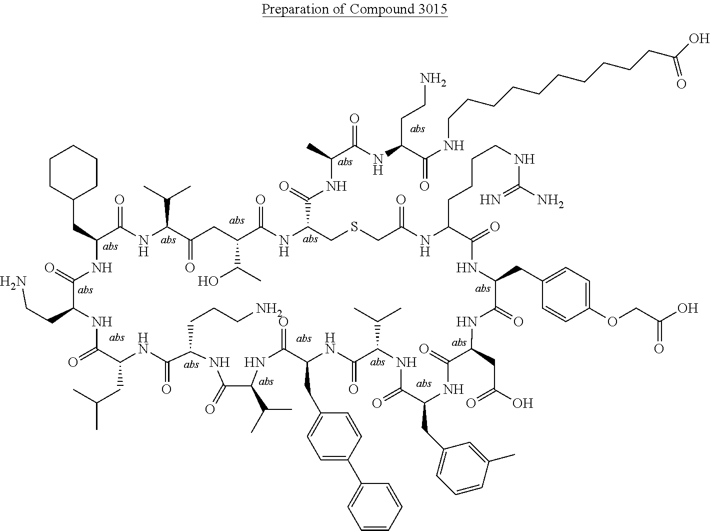 [SMILES: Cc1cccc(C[C@@H]2NC(=O)[C@H](CC(=O)O)NC(=O)[C@H](Cc3ccc(OCC(=O)O)cc3)NC(=O)C(CCCCNC(=N)N)NC(=O)CSC[C@@H](C(=O)N[C@@H](C)C(=O)N[C@@H](CCN)C(=O)NCCCCCCCCCCC(=O)O)NC(=O)[C@H](C(C)O)CC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3015 was prepared on a 25 μmol scale. The yield of the product was 4.8 mg, and its estimated purity by LCMS analysis was 95.1%. Analysis condition A: Retention time=1.69 min; ESI-MS(+) m/z [M+2H]²⁺: 1144.

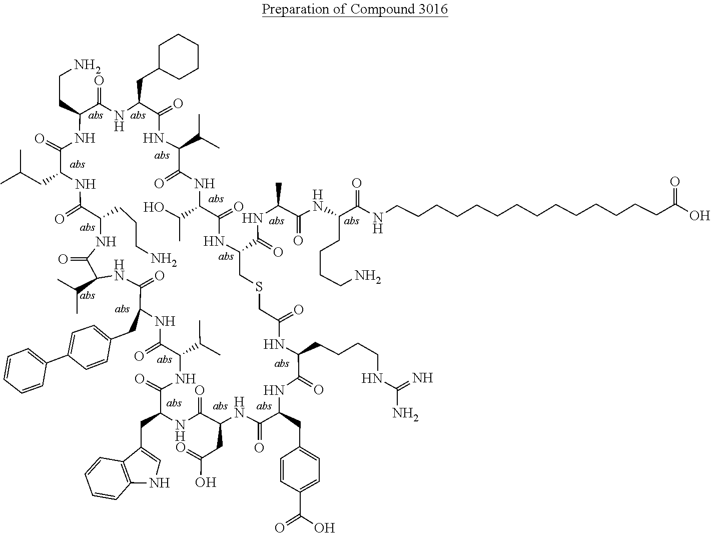 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CCCCNC(=N)N)NC(=O)CSC[C@@H](C(=O)N[C@@H](C)C(=O)N[C@@H](CCCCN)C(=O)NCCCCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3016 was prepared on a 25 μmol scale. The yield of the product was 11.4 mg, and its estimated purity by LCMS analysis was 96.1%. Analysis condition B: Retention time=1.79 min; ESI-MS(+) m/z [M+2H]²⁺: 1183.

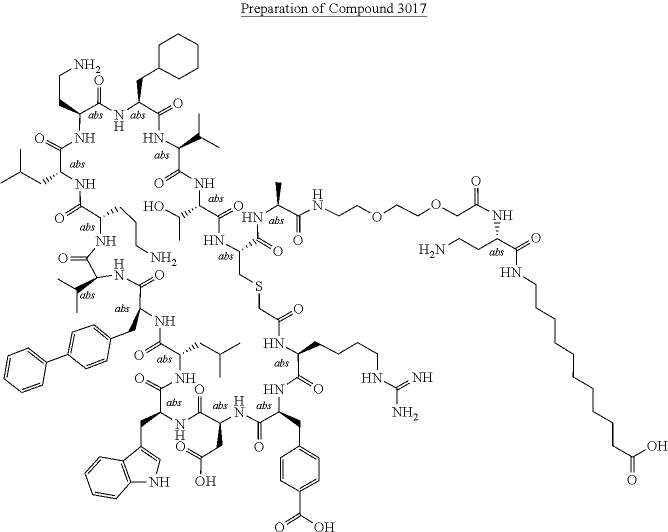 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](CCCCNC(=N)N)NC(=O)CSC[C@@H](C(=O)N[C@@H](C)C(=O)NCCOCCOCC(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3017 was prepared on a 25 μmol scale. The yield of the product was 15.8 mg, and its estimated purity by LCMS analysis was 96%. Analysis condition A: Retention time=1.67 min; ESI-MS(+) m/z [M+3H]³⁺: 809.2.

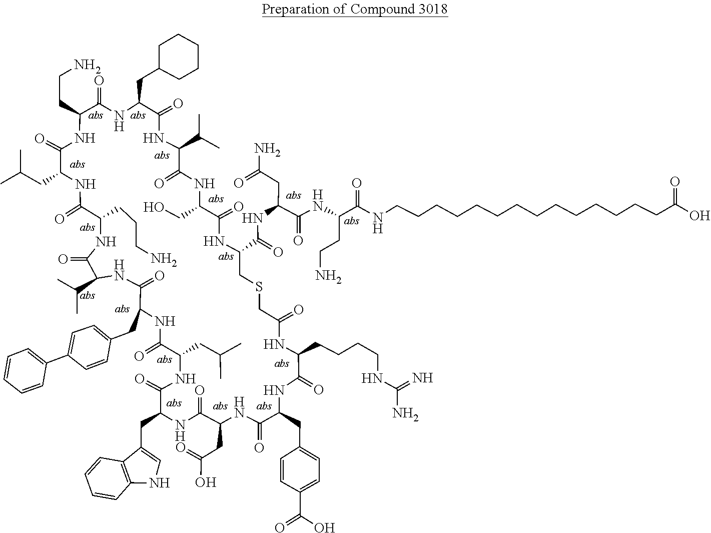

Compound 3018 was prepared on a 25 μmol scale. The yield of the product was 8.4 mg, and its estimated purity by LCMS analysis was 98.9%. Analysis condition B: Retention time=1.81 min; ESI-MS(+) m/z [M+2H]²⁺: 1191.1.

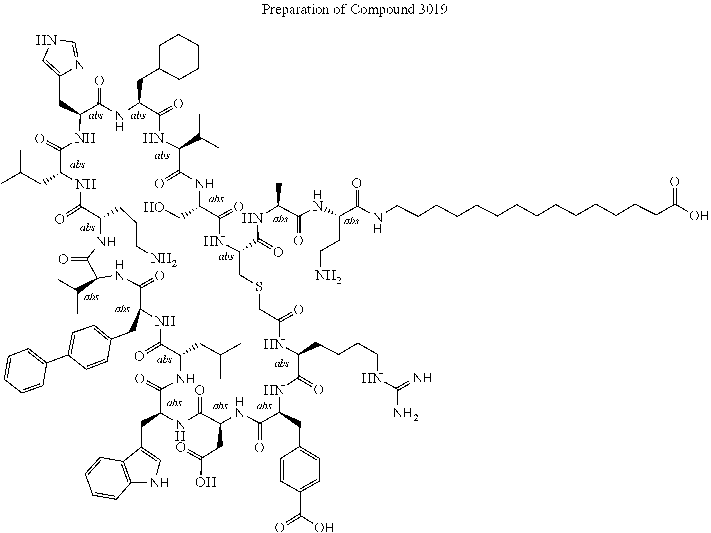

Compound 3019 was prepared on a 25 μmol scale. The yield of the product was 9.8 mg, and its estimated purity by LCMS analysis was 95.7%. Analysis condition A: Retention time=1.96 min; ESI-MS(+) m/z [M+3H]³⁺: 792.3.

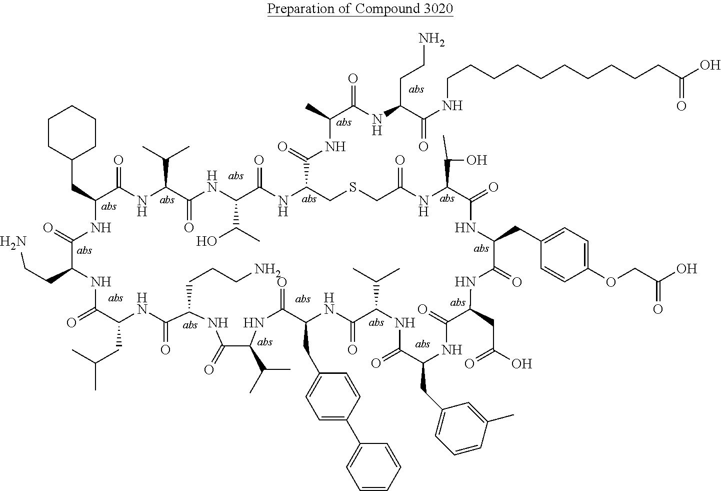 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](C)C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3020 was prepared on a 25 μmol scale. The yield of the product was 6.7 mg, and its estimated purity by LCMS analysis was 100%. Analysis condition A: Retention time=1.45 min; ESI-MS(+) m/z [M+2H]²⁺: 1109.1.

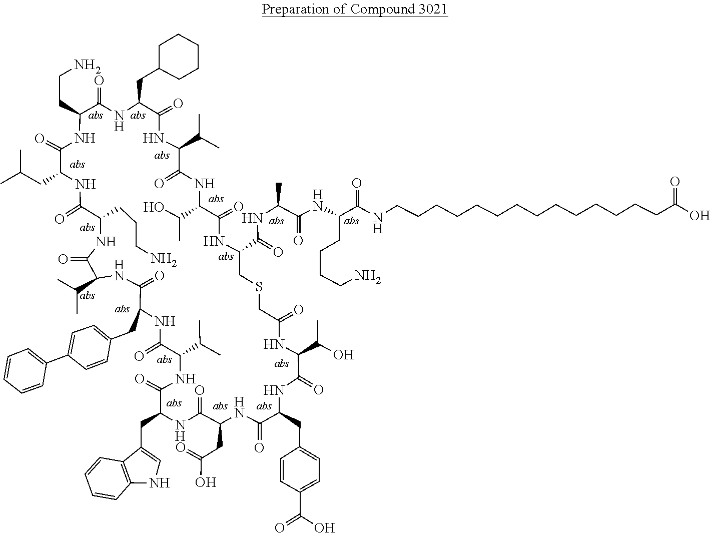 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](C)C(=O)N[C@@H](CCCCN)C(=O)NCCCCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3021 was prepared on a 25 μmol scale. The yield of the product was 12.4 mg, and its estimated purity by LCMS analysis was 100%. Analysis condition A: Retention time=1.57 min; ESI-MS(+) m/z [M+2H]²⁺: 1147.9.

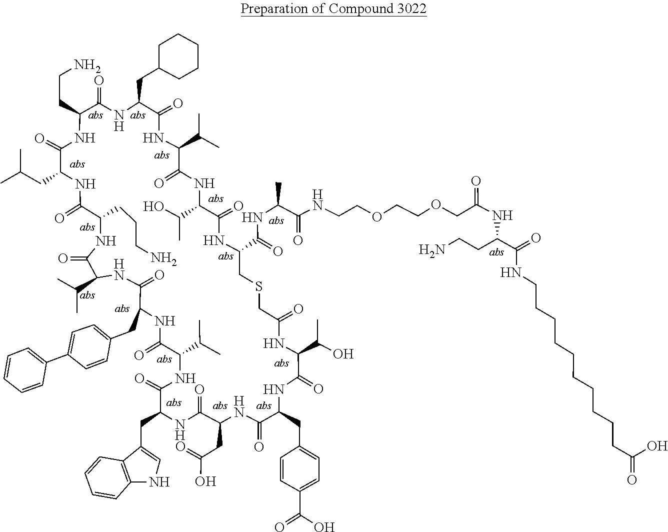 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](C)C(=O)NCCOCCOCC(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3022 was prepared on a 25 μmol scale. The yield of the product was 18.3 mg, and its estimated purity by LCMS analysis was 98.4%. Analysis condition B: Retention time=1.71 min; ESI-MS(+) m/z [M+2H]²⁺: 1179.

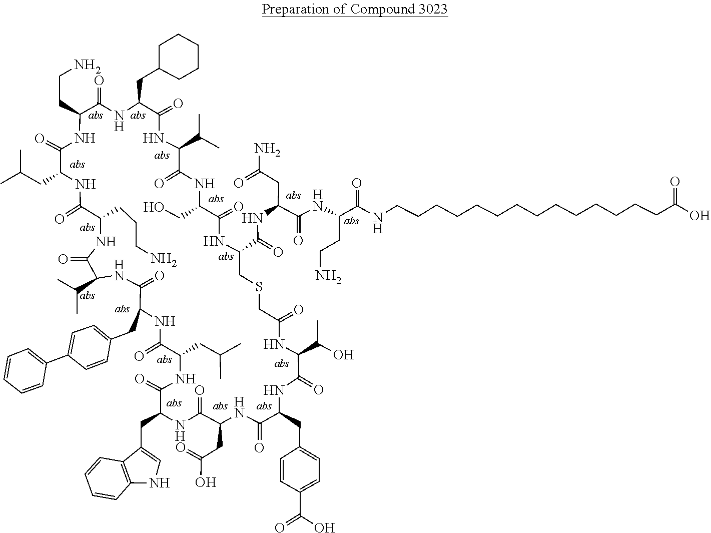

Compound 3023 was prepared on a 25 μmol scale. The yield of the product was 3 mg, and its estimated purity by LCMS analysis was 100%. Analysis condition A: Retention time=1.59 min; ESI-MS(+) m/z [M+2H]²⁺: 1156.2.

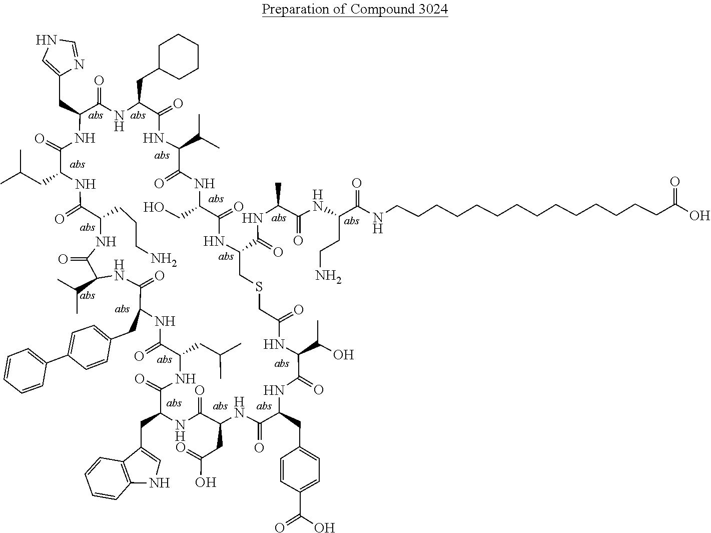

Compound 3024 was prepared on a 25 μmol scale. The yield of the product was 4.6 mg, and its estimated purity by LCMS analysis was 98.2%. Analysis condition A: Retention time=1.95, 1.99 min; ESI-MS(+) m/z [M+2H]²⁺: 1153.1, 1153.1.

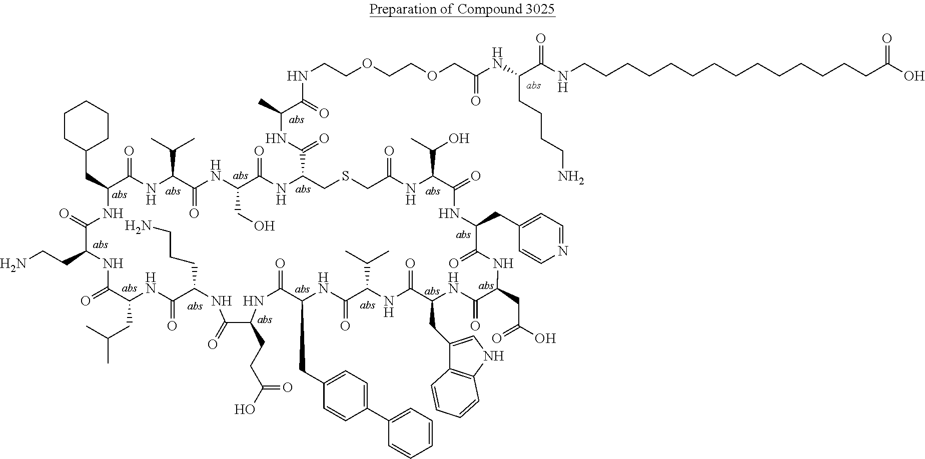 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](C(C)O)NC(=O)CSC[C@@H](C(=O)N[C@@H](C)C(=O)NCCOCCOCC(=O)N[C@@H](CCCCN)C(=O)NCCCCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3025 was prepared on a 25 μmol scale. The yield of the product was 19.4 mg, and its estimated purity by LCMS analysis was 96.7%. Analysis condition B: Retention time=1.41 min; ESI-MS(+) m/z [M+2H]²⁺: 1207.

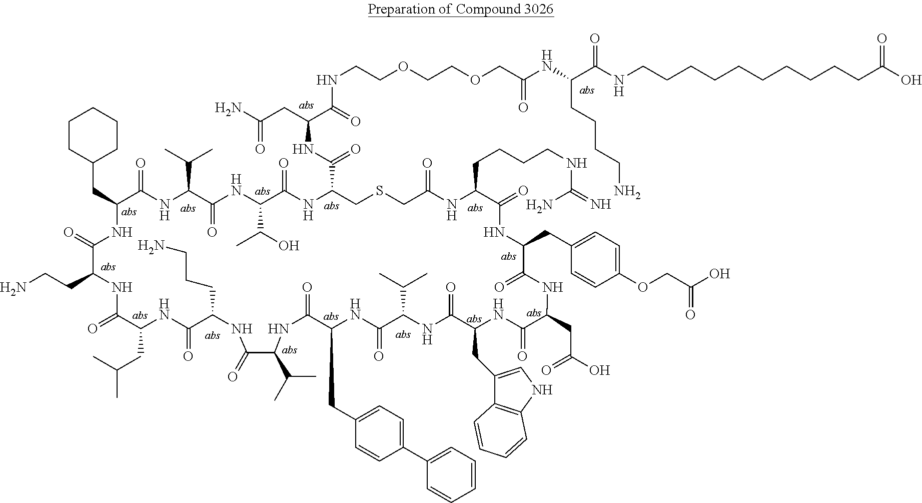 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CC(N)=O)C(=O)NCCOCCOCC(=O)N[C@@H](CCCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3026 was prepared on a 25 μmol scale. The yield of the product was 8.2 mg, and its estimated purity by LCMS analysis was 97%. Analysis condition A: Retention time=1.57 min; ESI-MS(+) m/z [M+3H]³⁺: 843.1.

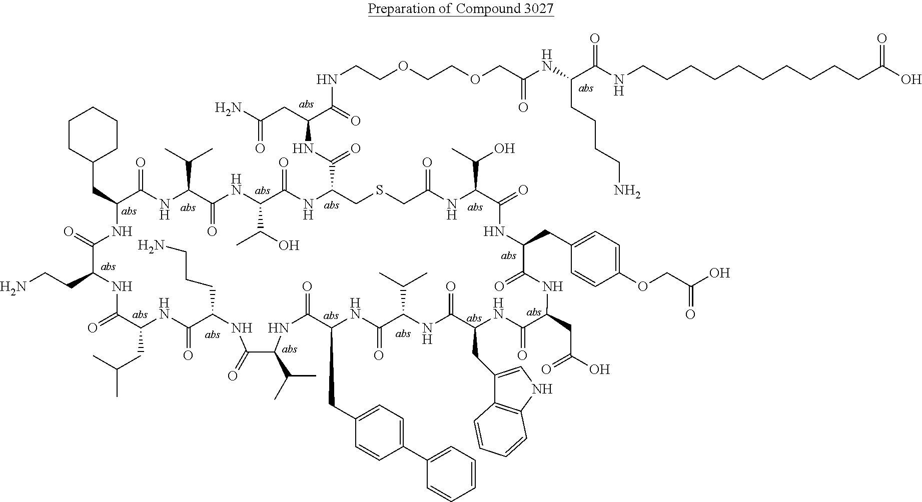 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)NCCOCCOCC(=O)N[C@@H](CCCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3027 was prepared on a 25 μmol scale. The yield of the product was 6.6 mg, and its estimated purity by LCMS analysis was 95%. Analysis condition B: Retention time=1.48 min; ESI-MS(+) m/z [M+3H]³⁺: 820.1.

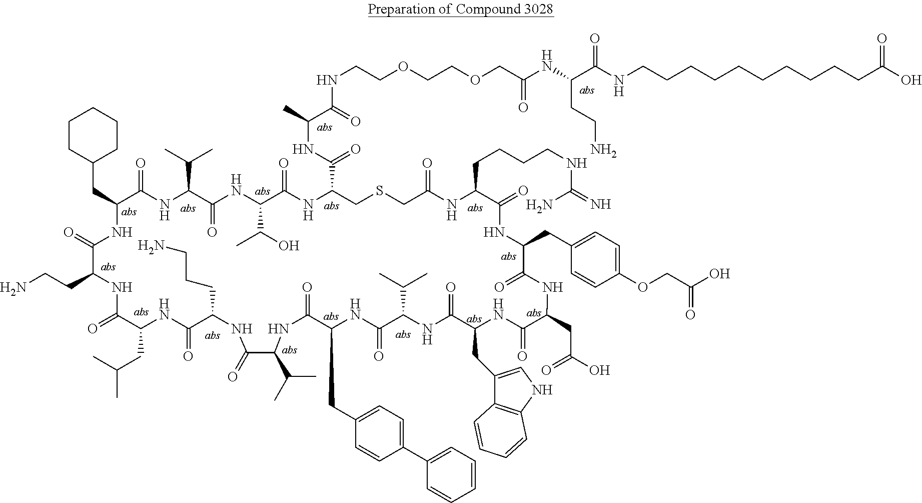 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](C)C(=O)NCCOCCOCC(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3028 was prepared on a 25 μmol scale. The yield of the product was 16.7 mg, and its estimated purity by LCMS analysis was 100%. Analysis condition A: Retention time=1.36 min; ESI-MS(+) m/z [M+2H]²⁺: 1228.3.

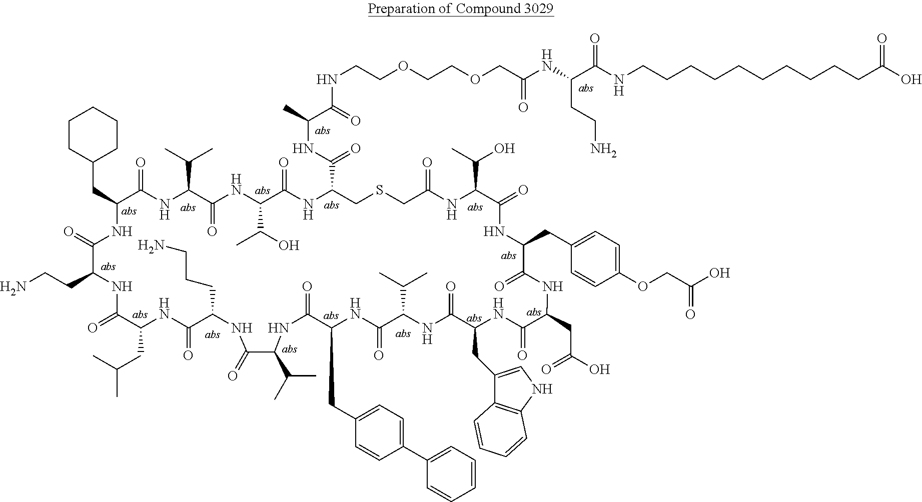 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](C)C(=O)NCCOCCOCC(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3029 was prepared on a 25 μmol scale. The yield of the product was 20.7 mg, and its estimated purity by LCMS analysis was 99.4%. Analysis condition A: Retention time=1.4 min; ESI-MS(+) m/z [M+2H]²⁺: 1194.2.

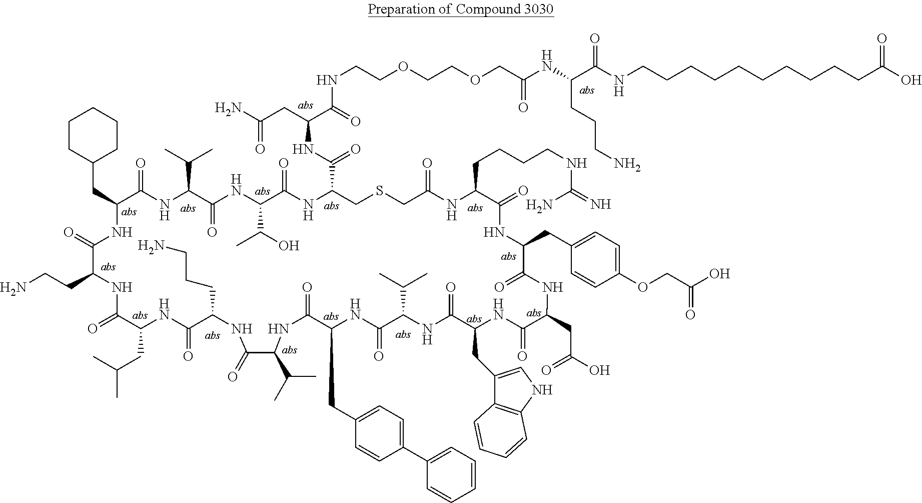 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CC(N)=O)C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3030 was prepared on a 25 μmol scale. The yield of the product was 13.3 mg, and its estimated purity by LCMS analysis was 95.1%. Analysis condition A: Retention time=1.62 min; ESI-MS(+) m/z [M+2H]²⁺: 1256.9.

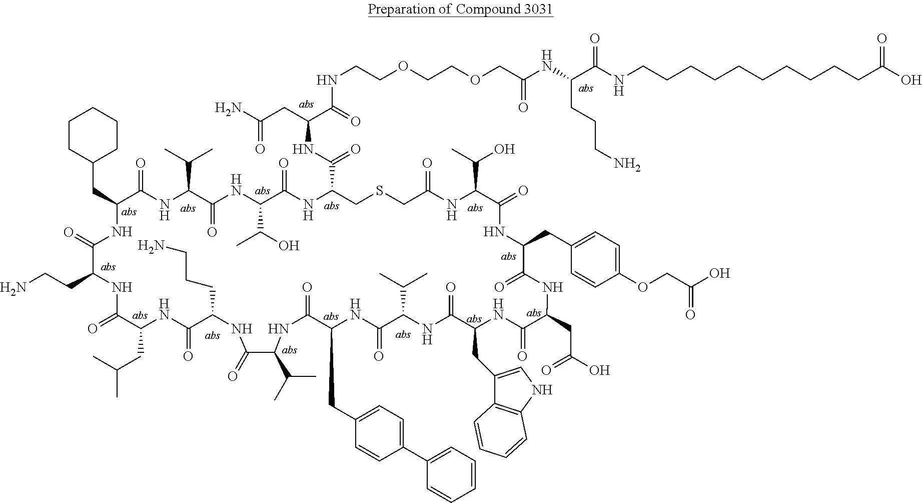 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3031 was prepared on a 25 μmol scale. The yield of the product was 7.6 mg, and its estimated purity by LCMS analysis was 97.2%. Analysis condition B: Retention time=1.39 min; ESI-MS(+) m/z [M+2H]²⁺: 1222.4.

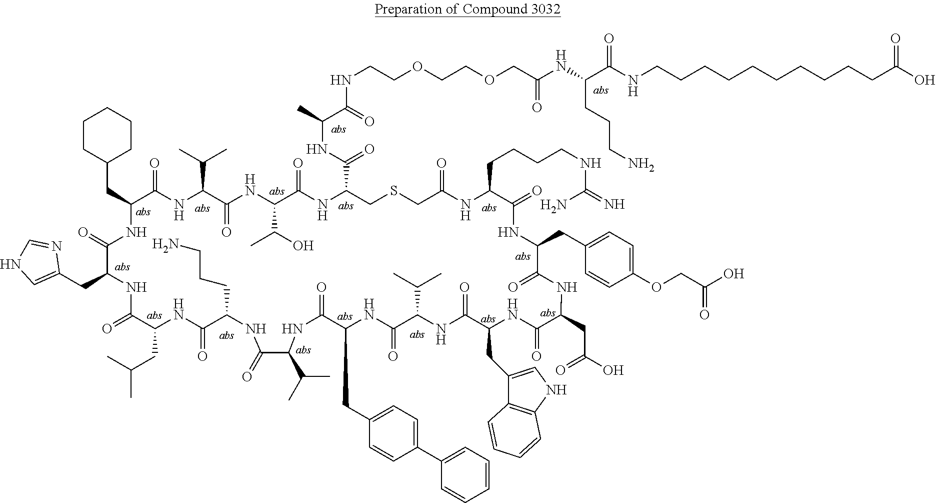 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](C)C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](Cc2c[nH]cn2)NC1=O]

Compound 3032 was prepared on a 25 μmol scale. The yield of the product was 34.9 mg, and its estimated purity by LCMS analysis was 100%. Analysis condition A: Retention time=1.38 min; ESI-MS(+) m/z [M+2H]²⁺: 1254.2.

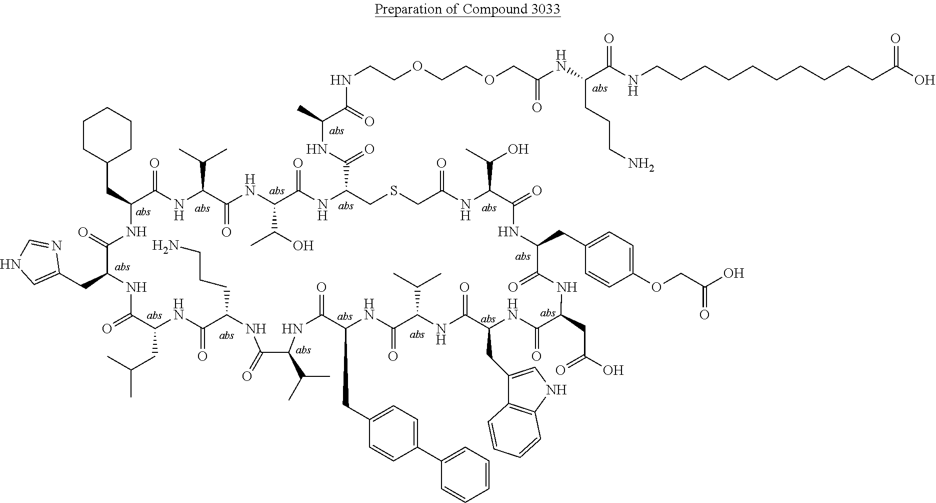 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](C)C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](Cc2c[nH]cn2)NC1=O]

Compound 3033 was prepared on a 25 μmol scale. The yield of the product was 28.2 mg, and its estimated purity by LCMS analysis was 95.7%. Analysis condition B: Retention time=1.39 min; ESI-MS(+) m/z [M+2H]²⁺: 1219.2.

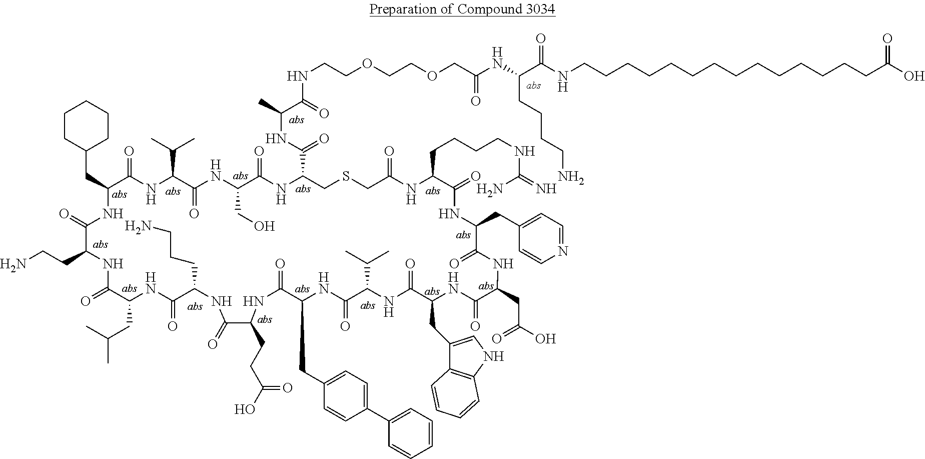 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CCCCNC(=N)N)NC(=O)CSC[C@@H](C(=O)N[C@@H](C)C(=O)NCCOCCOCC(=O)N[C@@H](CCCCN)C(=O)NCCCCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3034 was prepared on a 25 μmol scale. The yield of the product was 18.9 mg, and its estimated purity by LCMS analysis was 97.8%. Analysis condition B: Retention time-1.43 min; ESI-MS(+) m/z [M+3H]³⁺: 828.1.

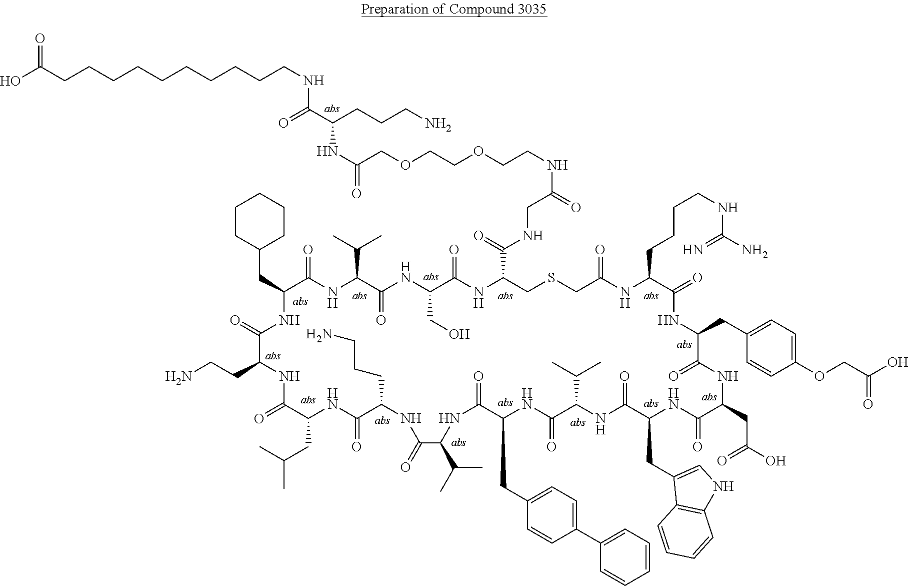 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3035 was prepared on a 50 μmol scale. The yield of the product was 13.3 mg, and its estimated purity by LCMS analysis was 96.5%. Analysis condition B: Retention time=1.5 min; ESI-MS(+) m/z [M+3H]³⁺: 815.2.

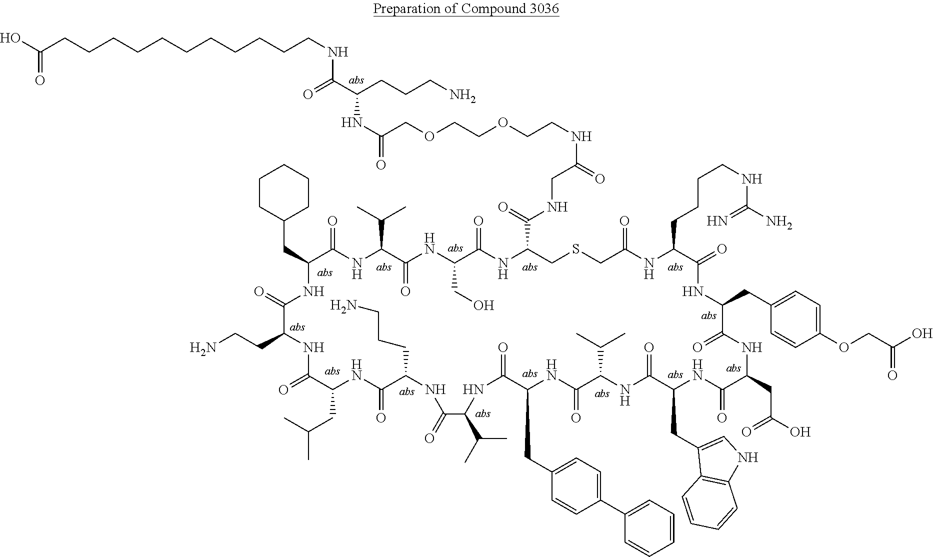 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3036 was prepared on a 50 μmol scale. The yield of the product was 14 mg, and its estimated purity by LCMS analysis was 96.6%. Analysis condition B: Retention time=1.57 min; ESI-MS(+) m/z [M+3H]³⁺: 819.1.

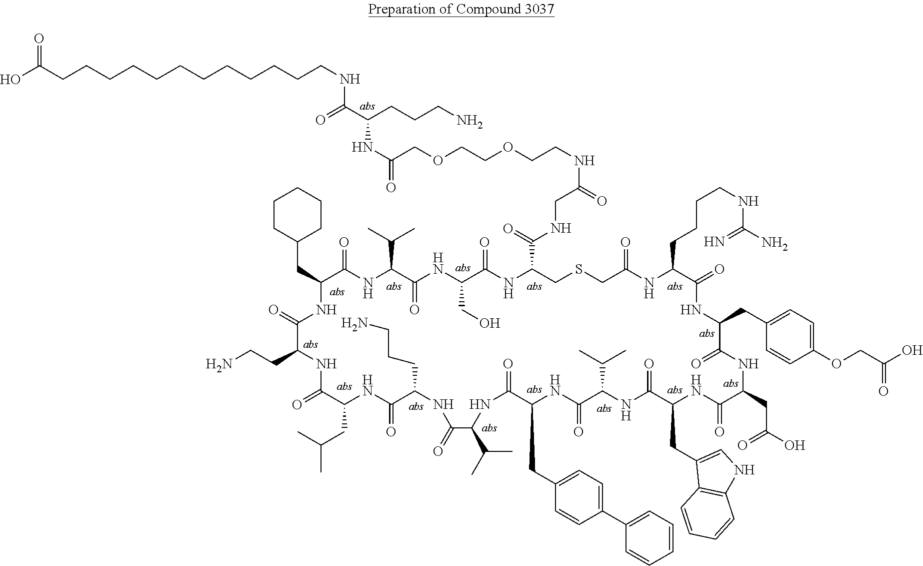 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3037 was prepared on a 50 μmol scale. The yield of the product was 11.3 mg, and its estimated purity by LCMS analysis was 97.1%. Analysis condition B: Retention time=1.61 min; ESI-MS(+) m/z [M+2H]²⁺: 1236.

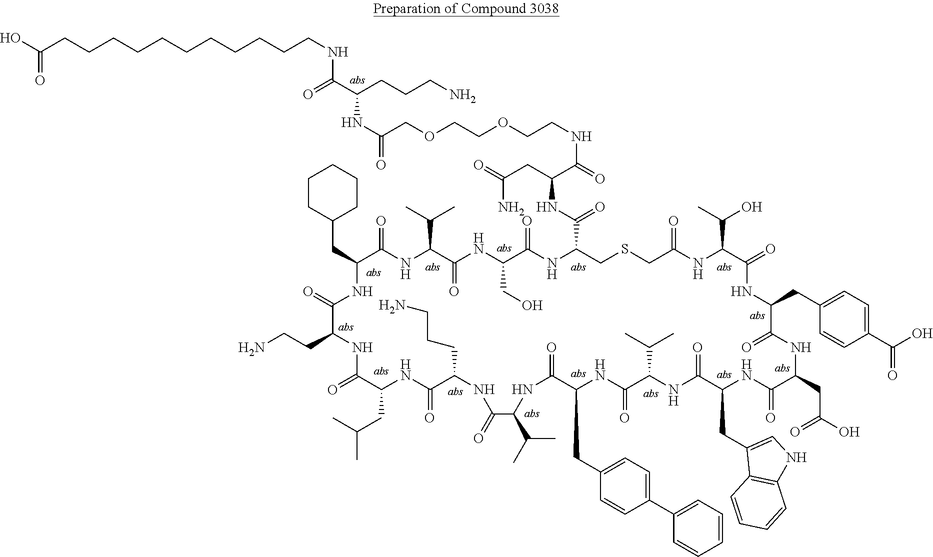 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CC(N)=O)C(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3038 was prepared on a 50 μmol scale. The yield of the product was 11.5 mg, and its estimated purity by LCMS analysis was 95%. Analysis condition A: Retention time=1.65 min; ESI-MS(+) m/z [M+3H]³⁺: 805.4.

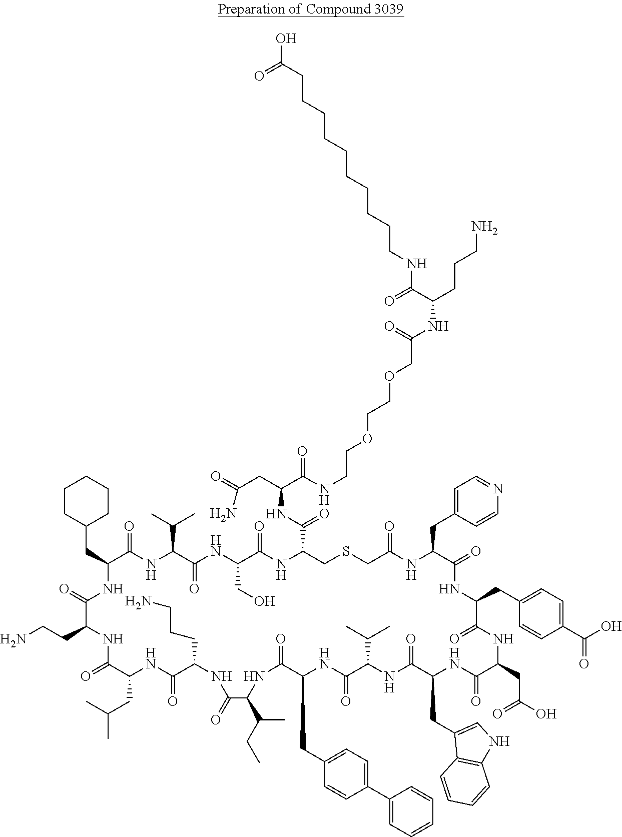 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CC(N)=O)C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3039 was prepared on a 50 μmol scale. The yield of the product was 6.8 mg, and its estimated purity by LCMS analysis was 100%. Analysis condition A: Retention time=1.71 min; ESI-MS(+) m/z [M+2H]²⁺: 1231.3.

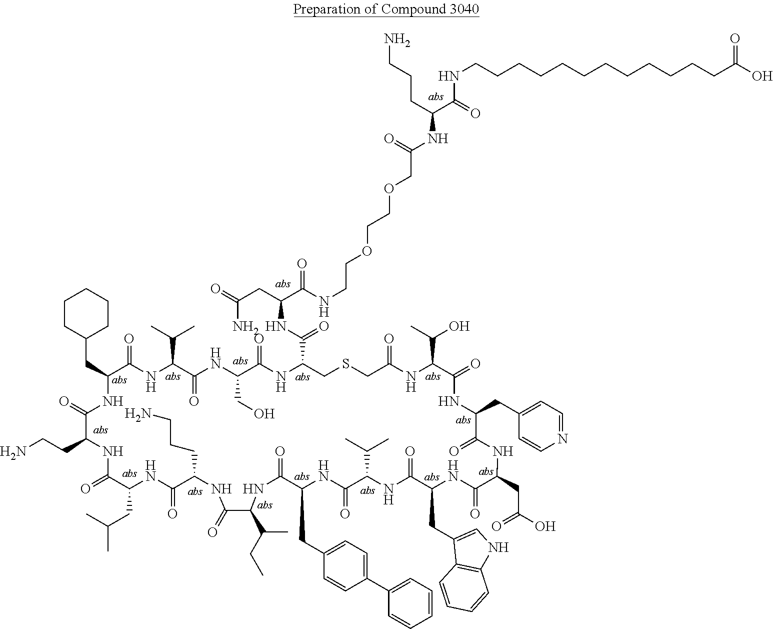 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](C(C)O)NC(=O)CSC[C@@H](C(=O)N[C@@H](CC(N)=O)C(=O)NCCOCCOCC(=O)N[C@@H](CCCN)C(=O)NCCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3040 was prepared on a 50 μmol scale. The yield of the product was 4.6 mg, and its estimated purity by LCMS analysis was 97.3%. Analysis condition B: Retention time=1.54 min; ESI-MS(+) m/z [M+2H]²⁺: 1200.1.

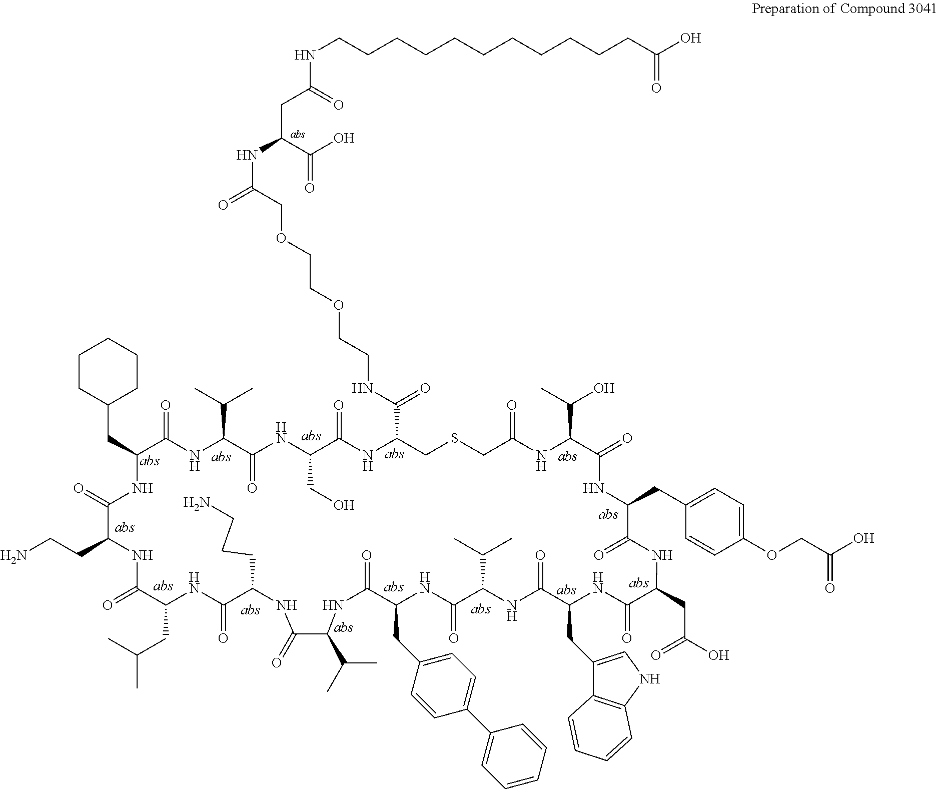 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)N[C@@H](CC(=O)NCCCCCCCCCCCC(=O)O)C(=O)O)NC(=O)[C@H](CO)NC(=O)[C@H](C(C)C)NC(=O)[C@H](CC2CCCCC2)NC(=O)[C@H](CCN)NC1=O]

Compound 3041 was prepared on a 50 μmol scale. The yield of the product was 16.7 mg, and its estimated purity by LCMS analysis was 96.1%. Analysis condition B: Retention time=1.89 min; ESI-MS(+) m/z [M+2H]²⁺: 1166.

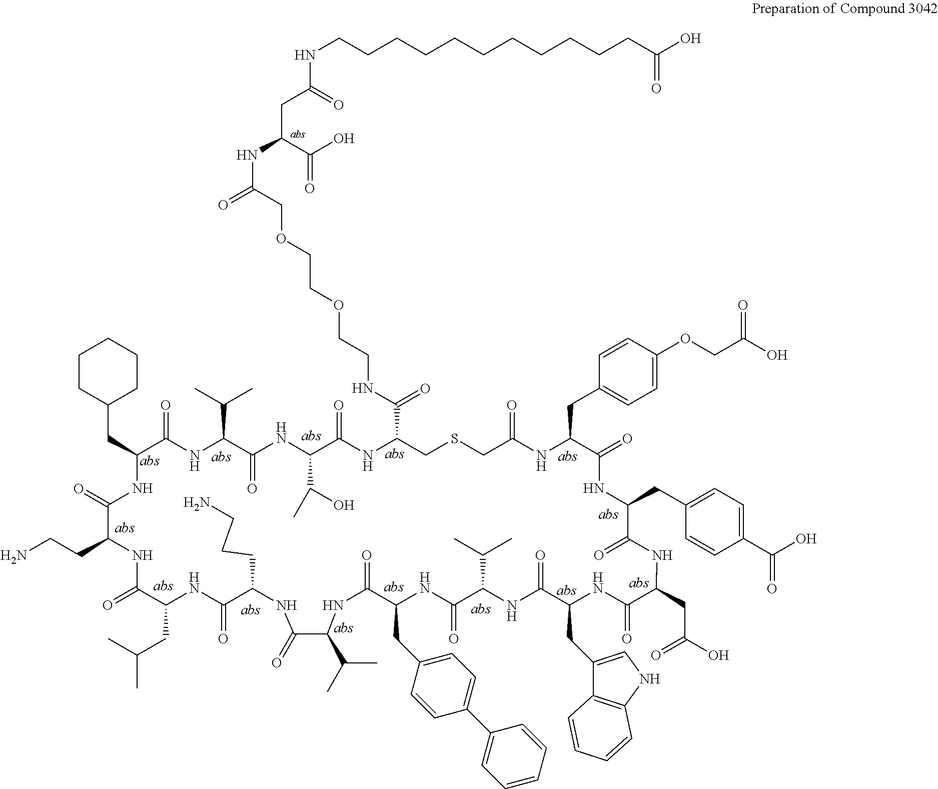 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCCOCCOCC(=O)N[C@@H](CC(=O)NCCCCCCCCCCCC(=O)O)C(=O)O)NC(=O)[C@H](C(C)O)NC(=O)[C@H](C(C)C)NC(=O)[C@H](CC2CCCCC2)NC(=O)[C@H](CCN)NC1=O]

Compound 3042 was prepared on a 50 μmol scale. The yield of the product was 26.4 mg, and its estimated purity by LCMS analysis was 95.4%. Analysis condition A: Retention time=1.69 min; ESI-MS(+) m/z [M+2H]²⁺: 1218.1.

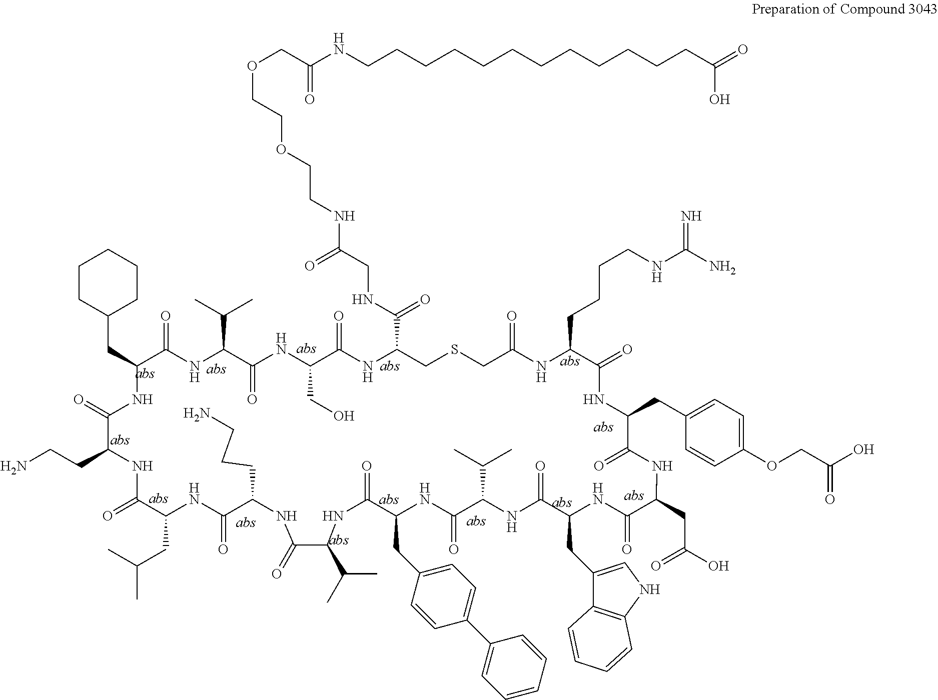 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)NCCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3043 was prepared on a 50 μmol scale. The yield of the product was 9.8 mg, and its estimated purity by LCMS analysis was 99.1%. Analysis condition B: Retention time=1.82 min; ESI-MS(+) m/z [M+2H]²⁺: 1179.

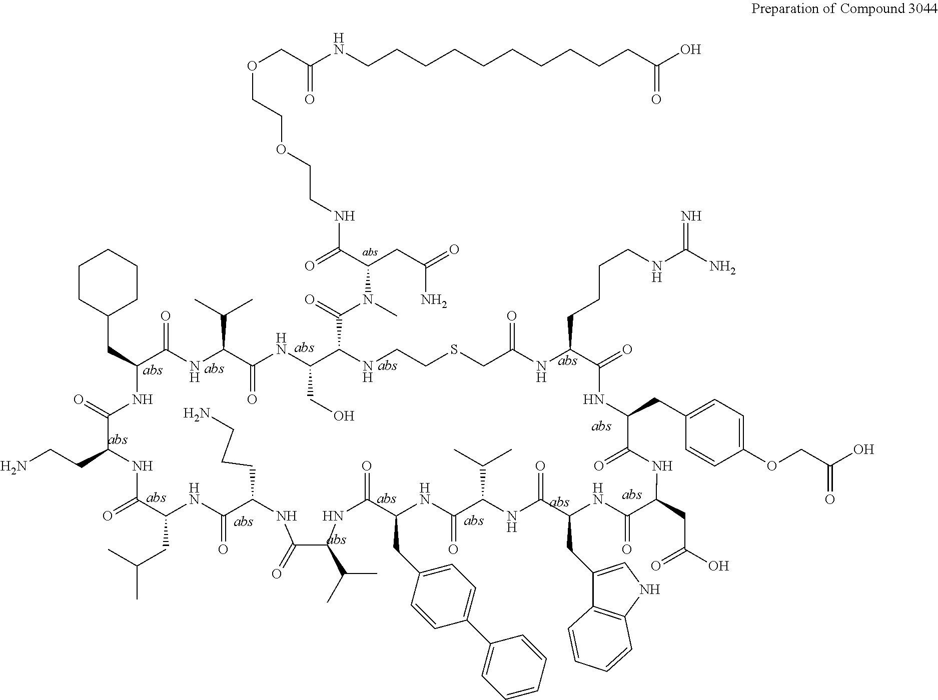 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSCCN[C@@H](C(=O)N(C)[C@@H](CC(N)=O)C(=O)NCCOCCOCC(=O)NCCCCCCCCCCC(=O)O)[C@H](CO)NC(=O)[C@H](C(C)C)NC(=O)[C@H](CC2CCCCC2)NC(=O)[C@H](CCN)NC1=O]

Compound 3044 was prepared on a 50 μmol scale. The yield of the product was 6.1 mg, and its estimated purity by LCMS analysis was 98.7%. Analysis condition B: Retention time=1.63 min; ESI-MS(+) m/z [M+2H]²⁺: 1200.1.

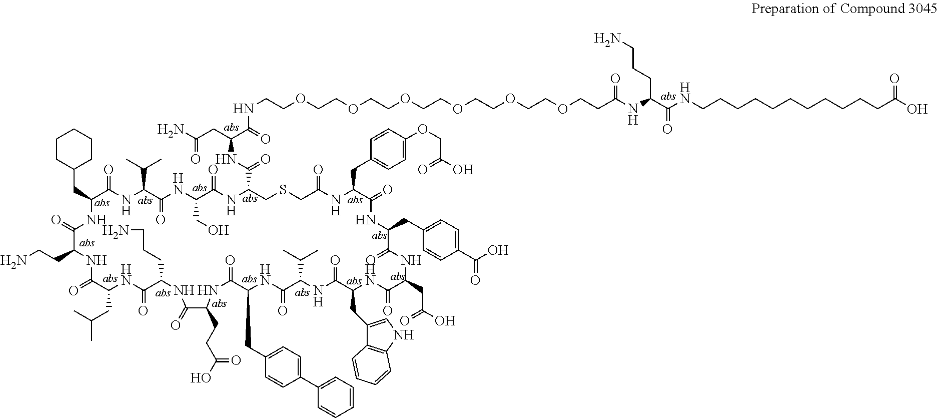 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CC(N)=O)C(=O)NCCOCCOCCOCCOCCOCCOCCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3045 was prepared on a 25 μmol scale. The yield of the product was 7.4 mg, and its estimated purity by LCMS analysis was 100%. Analysis condition A: Retention time=1.53 min; ESI-MS(+) m/z [M+3H]³⁺: 918.7.

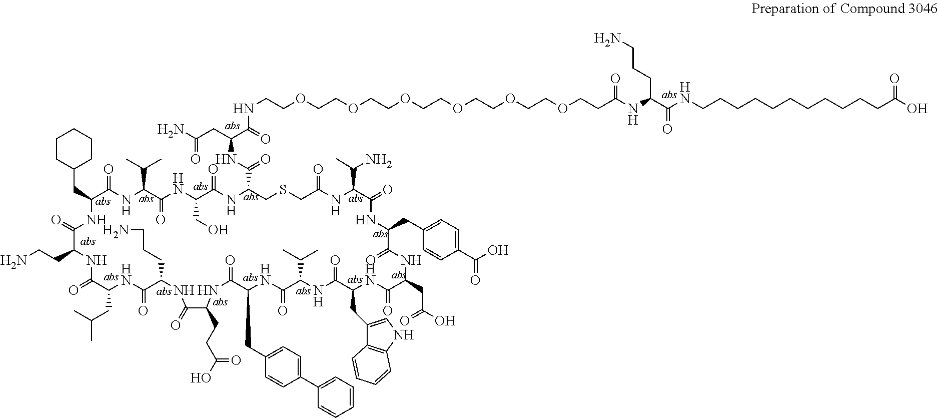 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](C(C)N)NC(=O)CSC[C@@H](C(=O)N[C@@H](CC(N)=O)C(=O)NCCOCCOCCOCCOCCOCCOCCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3046 was prepared on a 25 μmol scale. The yield of the product was 3.8 mg, and its estimated purity by LCMS analysis was 96.2%. Analysis condition A: Retention time=1.6 min; ESI-MS(+) m/z [M+2H]²⁺: 1317.

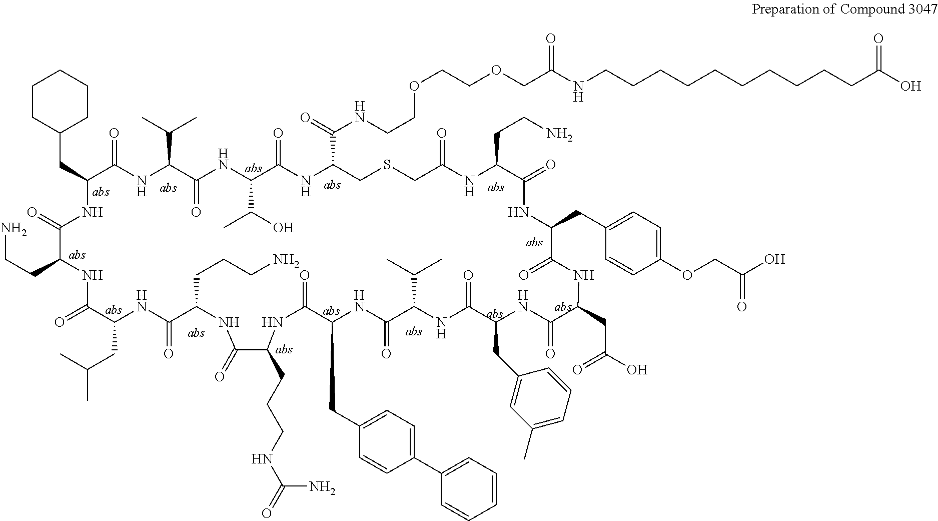 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3047 was prepared on a 50 μmol scale. The yield of the product was 29.4 mg, and its estimated purity by LCMS analysis was 98.5%. Analysis condition B: Retention time=1.69 min; ESI-MS(+) m/z [M+3H]³⁺: 750.1.

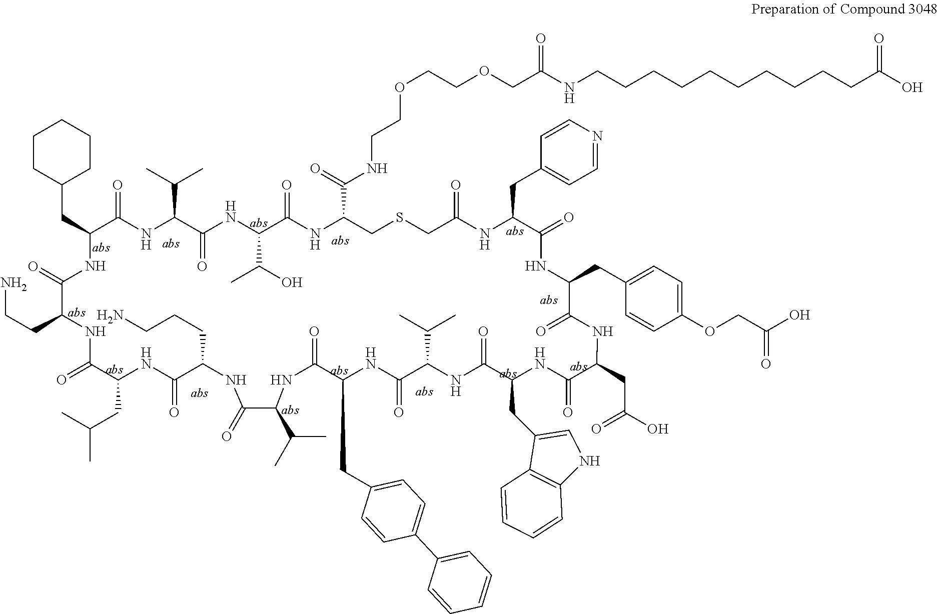 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3048 was prepared on a 50 μmol scale. The yield of the product was 38.1 mg, and its estimated purity by LCMS analysis was 95.9%. Analysis condition A: Retention time=1.77 min; ESI-MS(+) m/z [M+2H]²⁺: 1132.2.

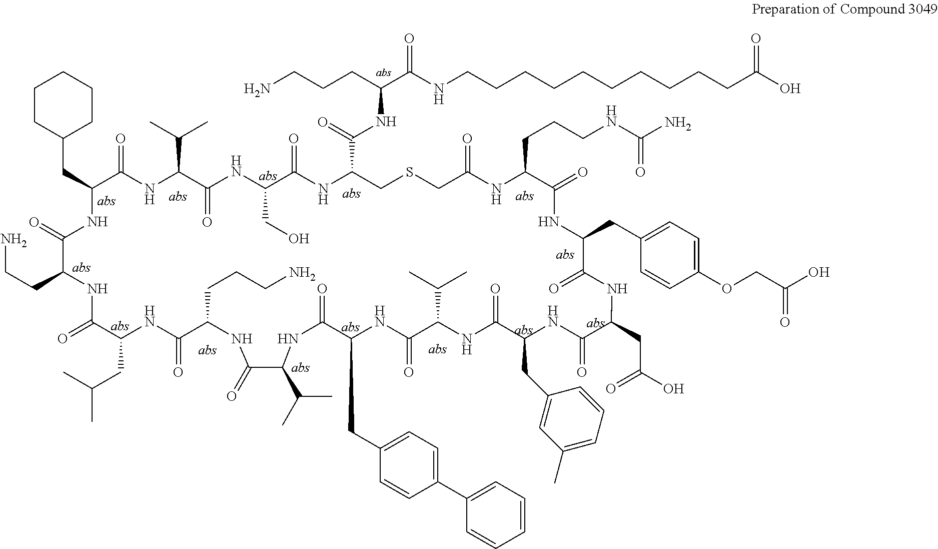 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(N)=O)NC(=O)CSC[C@@H](C(=O)N[C@@H](CCCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3049 was prepared on a 50 μmol scale. The yield of the product was 6.4 mg, and its estimated purity by LCMS analysis was 95.2%. Analysis condition B: Retention time=1.47 min; ESI-MS(+) m/z [M+3H]³⁺: 734.9.

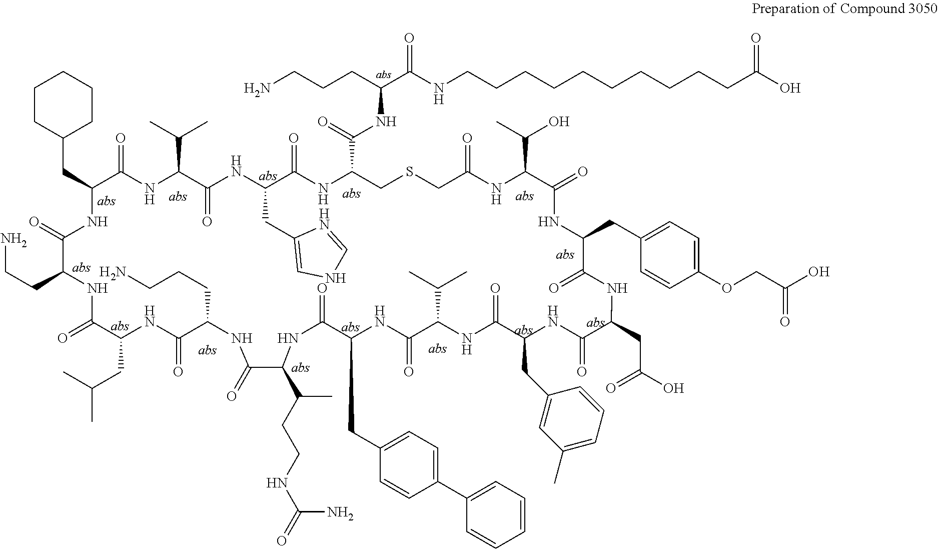

Compound 3050 was prepared on a 50 μmol scale. The yield of the product was 15.8 mg, and its estimated purity by LCMS analysis was 96.5%. Analysis condition B: Retention time=1.61 min; ESI-MS(+) m/z [M+2H]²⁺: 1127.3.

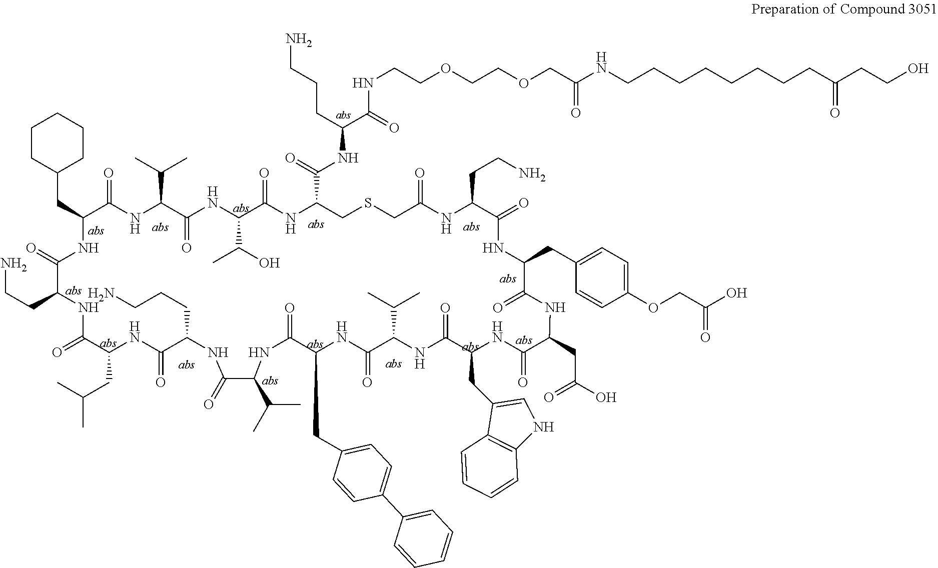 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCCN)C(=O)NCCOCCOCC(=O)NCCCCCCCCC(=O)CCO)NC(=O)[C@H](C(C)O)NC(=O)[C@H](C(C)C)NC(=O)[C@H](CC2CCCCC2)NC(=O)[C@H](CCN)NC1=O]

Compound 3051 was prepared on a 50 μmol scale. The yield of the product was 13.2 mg, and its estimated purity by LCMS analysis was 96.4%. Analysis condition A: Retention time=1.71 min; ESI-MS(+) m/z [M+2H]²⁺: 777.2.

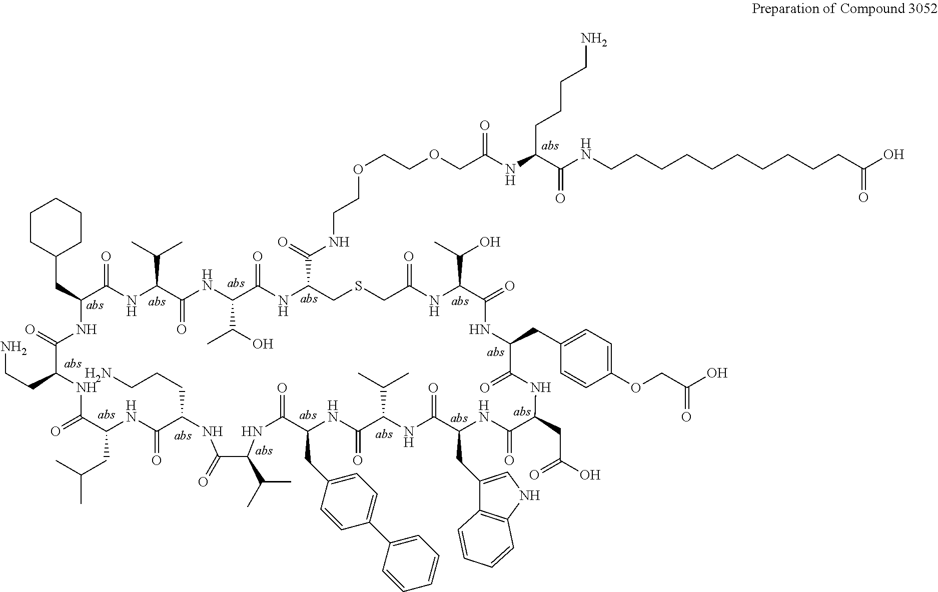 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)N[C@@H](CCCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3052 was prepared on a 50 μmol scale. The yield of the product was 29.5 mg, and its estimated purity by LCMS analysis was 100%. Analysis condition A: Retention time=1.76 min; ESI-MS(+) m/z [M+3H]³⁺: 782.1.

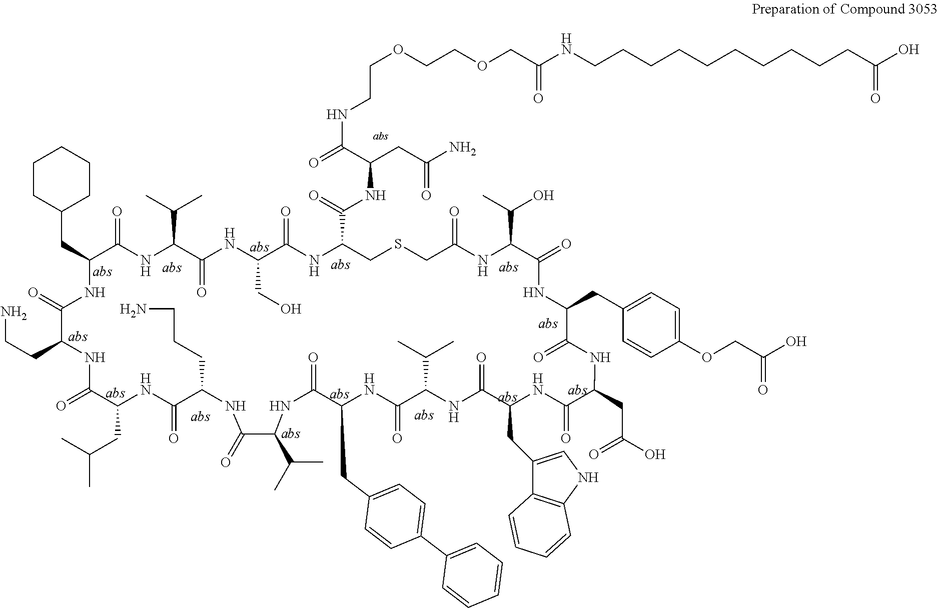 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3053 was prepared on a 50 μmol scale. The yield of the product was 3 mg, and its estimated purity by LCMS analysis was 98.3%. Analysis condition B: Retention time=1.78 min; ESI-MS(+) m/z [M+2H]²⁺: 1159.2.

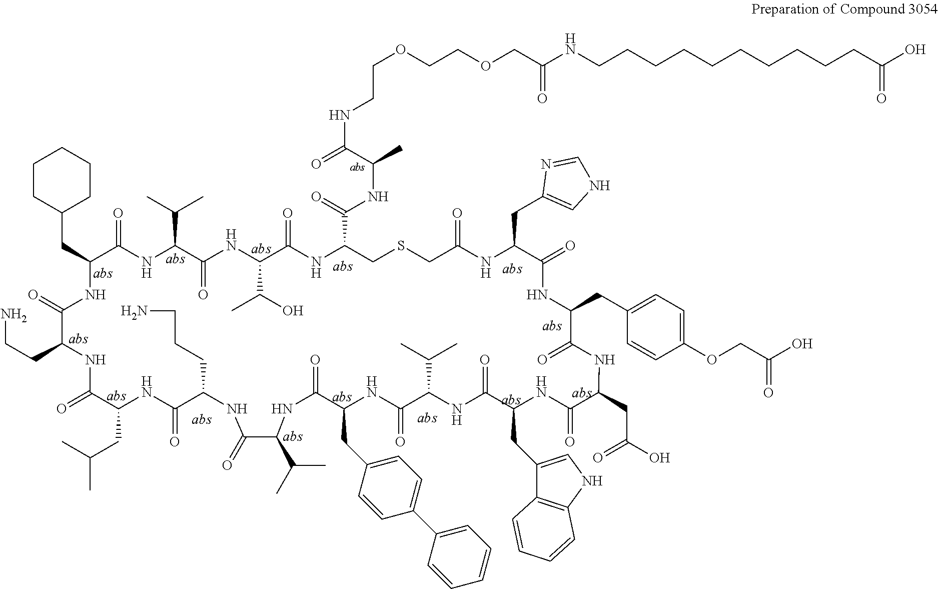 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2c[nH]cn2)NC(=O)CSC[C@@H](C(=O)N[C@H](C)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3054 was prepared on a 50 μmol scale. The yield of the product was 34 mg, and its estimated purity by LCMS analysis was 100%. Analysis condition B: Retention time=1.67 min; ESI-MS(+) m/z [M+2H]²⁺: 1162.1.

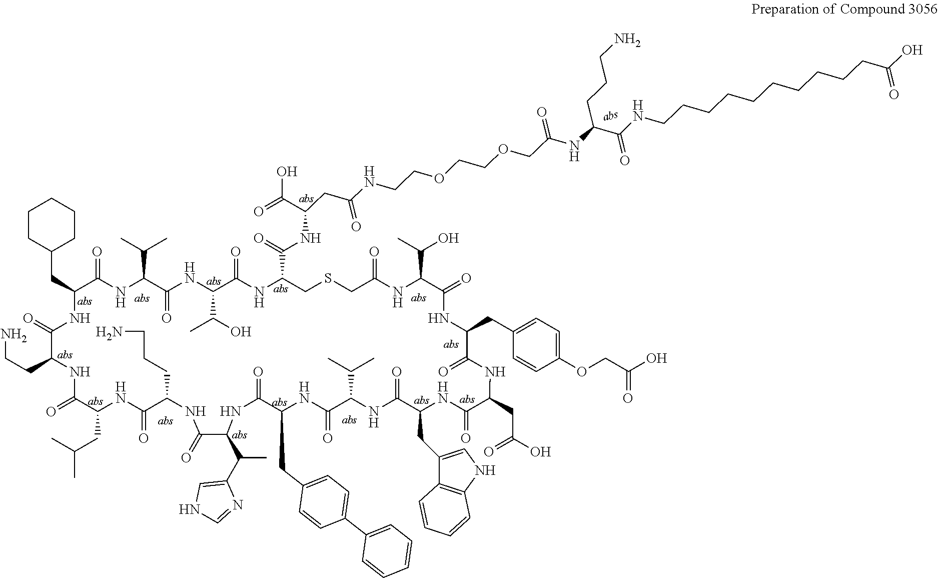 [SMILES: CC(C)C[C@H]1NC(=O)[C@H](CCCN)NC(=O)[C@H](C(C)c2c[nH]cn2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(=O)NCCOCCOCC(=O)N[C@@H](CCCN)C(=O)NCCCCCCCCCCC(=O)O)C(=O)O)NC(=O)[C@H](C(C)O)NC(=O)[C@H](C(C)C)NC(=O)[C@H](CC2CCCCC2)NC(=O)[C@H](CCN)NC1=O]

Compound 3055 was prepared on a 50 μmol scale. The yield of the product was 11.9 mg, and its estimated purity by LCMS analysis was 100%. Analysis condition A: Retention time=1.66 min; ESI-MS(+) m/z [M+2H]²⁺: 1240.9.

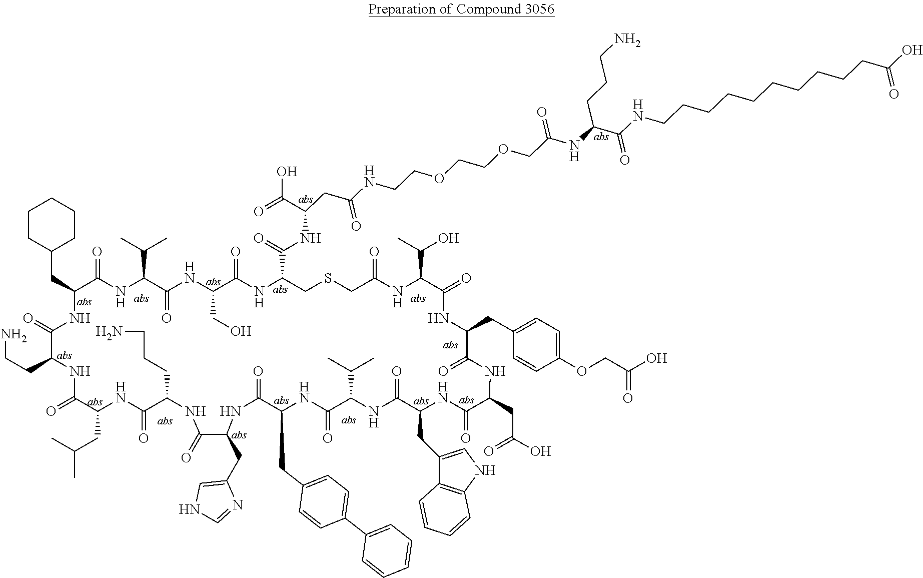 [SMILES: CC(C)C[C@H]1NC(=O)[C@H](CCCN)NC(=O)[C@H](Cc2c[nH]cn2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(=O)NCCOCCOCC(=O)N[C@@H](CCCN)C(=O)NCCCCCCCCCCC(=O)O)C(=O)O)NC(=O)[C@H](CO)NC(=O)[C@H](C(C)C)NC(=O)[C@H](CC2CCCCC2)NC(=O)[C@H](CCN)NC1=O]

Compound 3056 was prepared on a 50 μmol scale. The yield of the product was 20.9 mg, and its estimated purity by LCMS analysis was 100%. Analysis condition A: Retention time=1.58 min; ESI-MS(+) m/z [M+2H]²⁺: 1235.

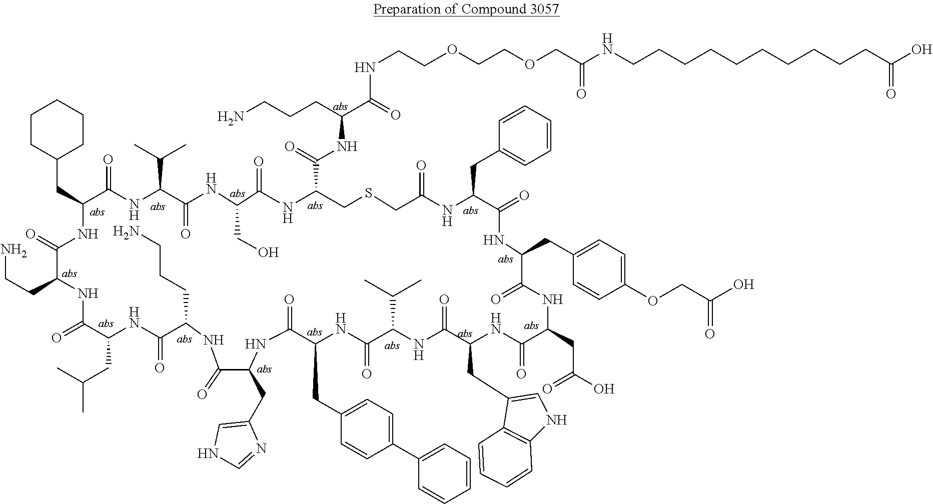 [SMILES: CC(C)C[C@H]1NC(=O)[C@H](CCCN)NC(=O)[C@H](Cc2c[nH]cn2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3057 was prepared on a 50 μmol scale. The yield of the product was 36.1 mg, and its estimated purity by LCMS analysis was 95.6%. Analysis condition B: Retention time=1.68 min; ESI-MS(+) m/z [M+3H]³⁺: 801.

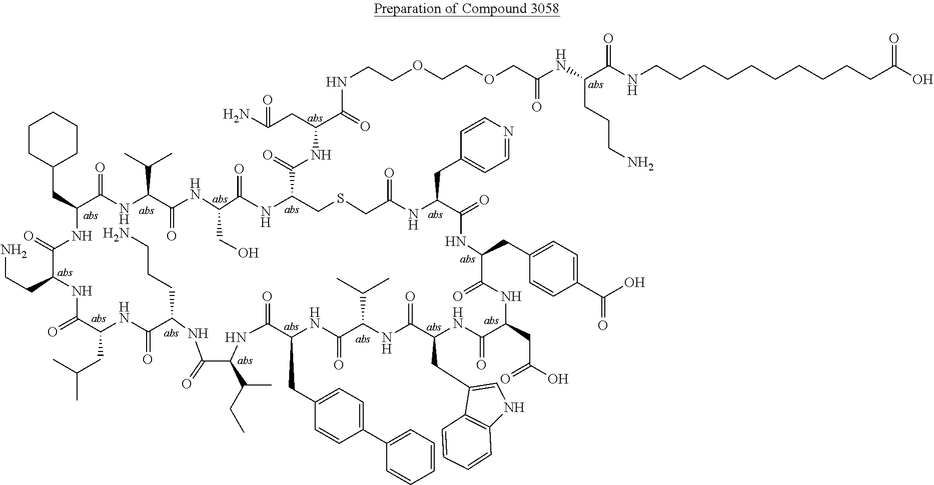 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@H](CC(N)=O)C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3058 was prepared on a 50 μmol scale. The yield of the product was 3.9 mg, and its estimated purity by LCMS analysis was 100%. Analysis condition B: Retention time=1.56 min; ESI-MS(+) m/z [M+2H]²⁺: 1230.9.

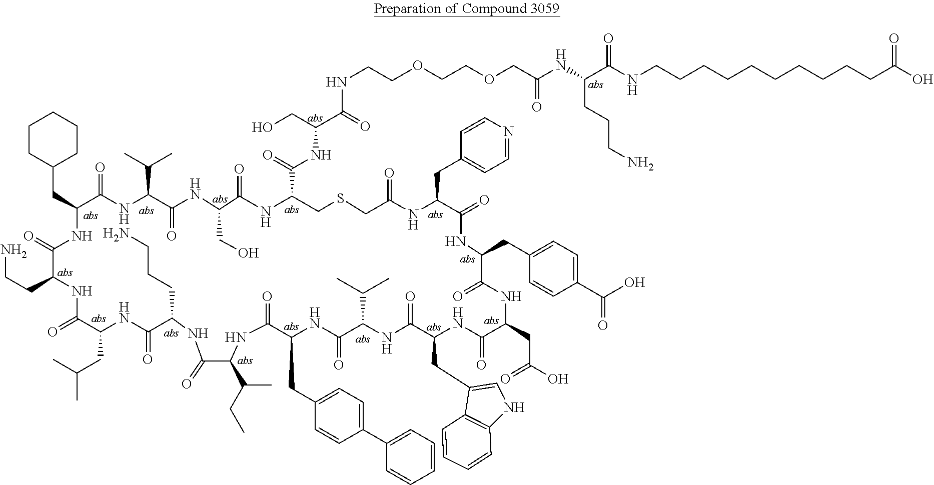 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@H](CO)C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3059 was prepared on a 50 μmol scale. The yield of the product was 14.3 mg, and its estimated purity by LCMS analysis was 95.3%. Analysis condition A: Retention time=1.76 min; ESI-MS(+) m/z [M+3H]³⁺: 812.2.

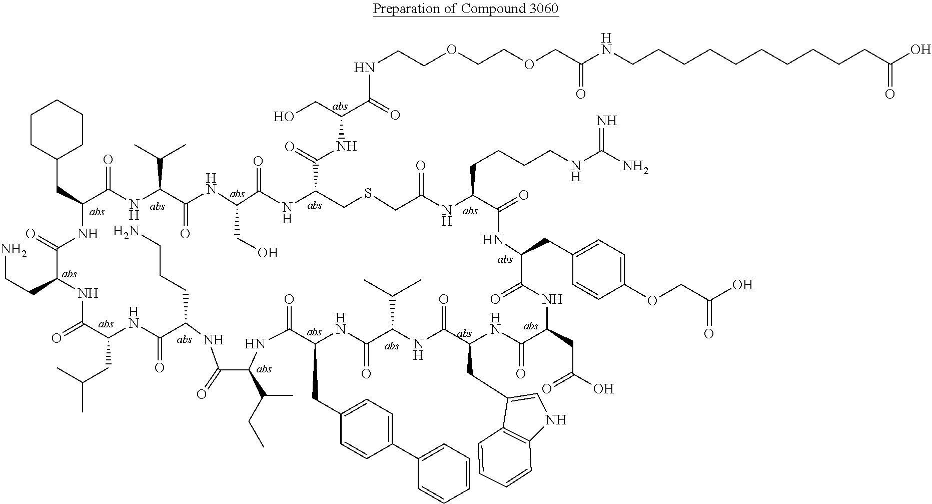 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@H](CO)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3060 was prepared on a 50 μmol scale. The yield of the product was 8.9 mg, and its estimated purity by LCMS analysis was 95.9%. Analysis condition B: Retention time=1.68 min; ESI-MS(+) m/z [M+2H]²⁺: 1186.3.

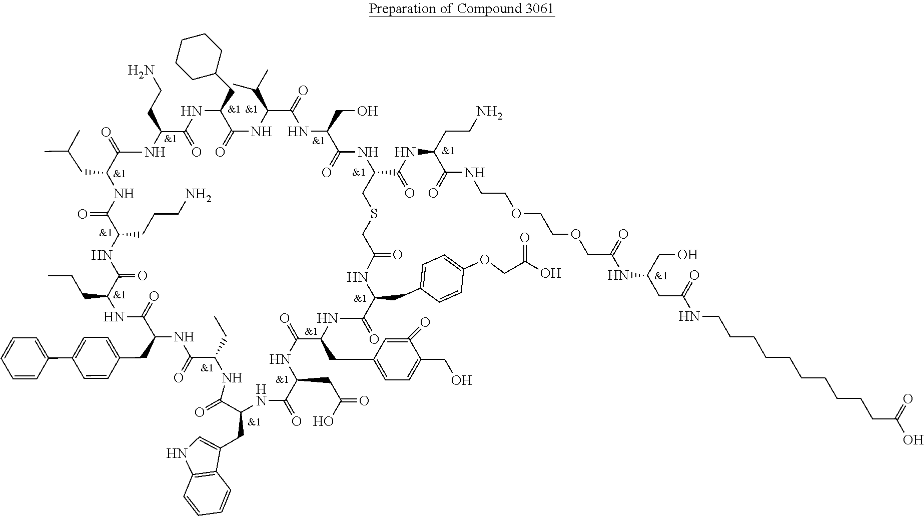

Compound 3061 was prepared on a 50 μmol scale. The yield of the product was 12.8 mg, and its estimated purity by LCMS analysis was 98.2%. Analysis condition A: Retention time=1.67 min; ESI-MS(+) m/z [M+2H]²⁺: 1260.3.

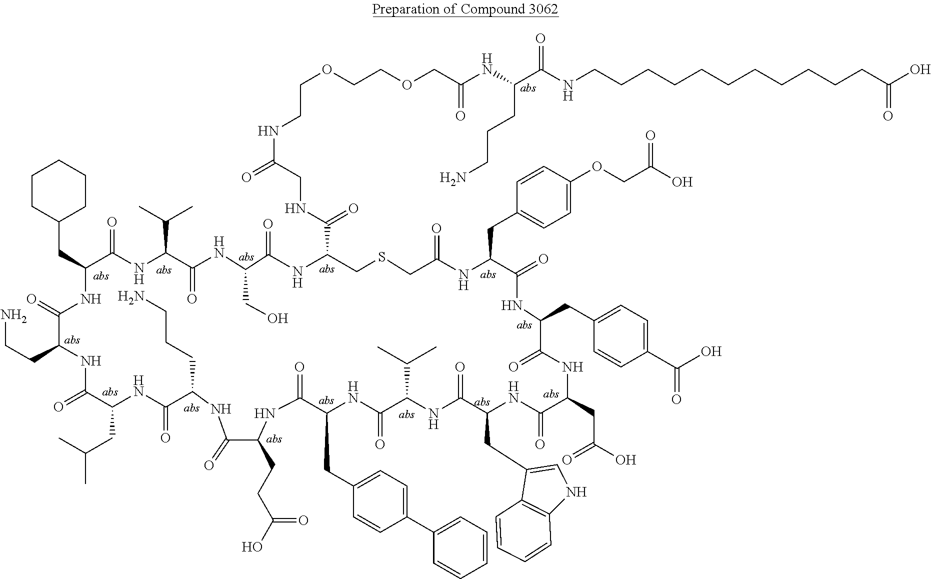 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3062 was prepared on a 50 μmol scale. The yield of the product was 17.1 mg, and its estimated purity by LCMS analysis was 97.3%. Analysis condition B: Retention time=1.64 min; ESI-MS(+) m/z [M+2H]²⁺: 1254.

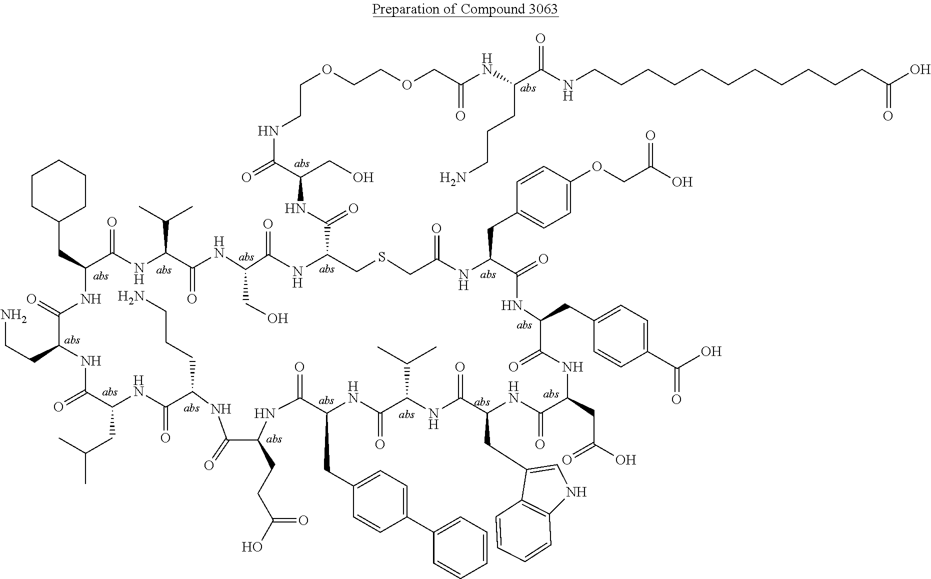 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@H](CO)C(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3063 was prepared on a 50 μmol scale. The yield of the product was 3.5 mg, and its estimated purity by LCMS analysis was 100%. Analysis condition A: Retention time=1.58 min; ESI-MS(+) m/z [M+2H]²⁺: 1269.

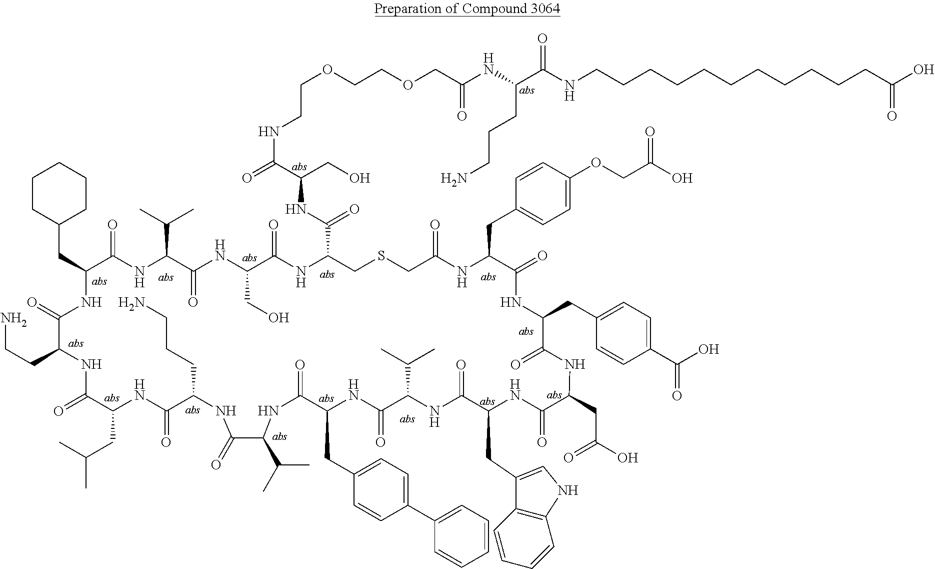 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@H](CO)C(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3064 was prepared on a 50 μmol scale. The yield of the product was 10.4 mg, and its estimated purity by LCMS analysis was 95.7%. Analysis condition B: Retention time=1.75 min; ESI-MS(+) m/z [M+2H]²⁺: 1254.2.

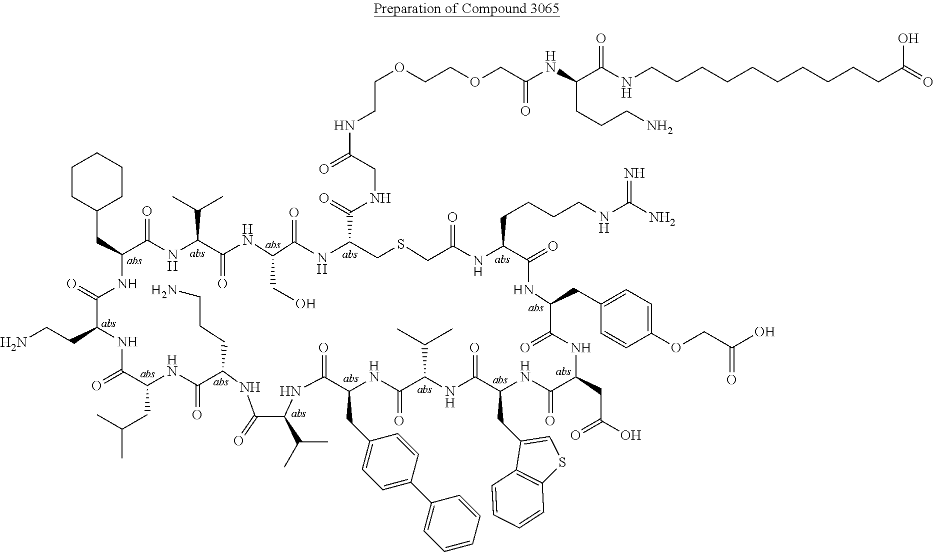 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2csc3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3065 was prepared on a 50 μmol scale. The yield of the product was 24.9 mg, and its estimated purity by LCMS analysis was 95%. Analysis condition B: Retention time=1.62 min; ESI-MS(+) m/z [M+2H]²⁺: 1230.4.

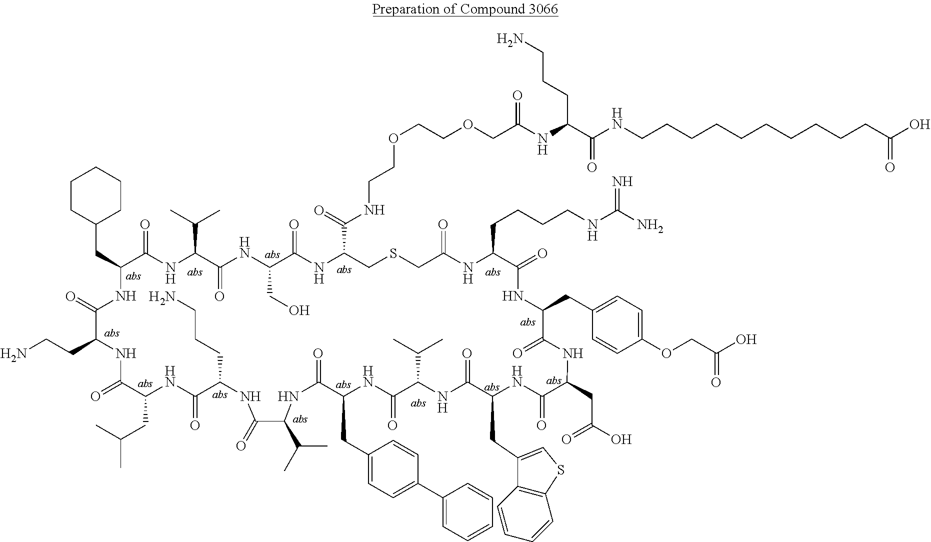 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2csc3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3066 was prepared on a 50 μmol scale. The yield of the product was 21.9 mg, and its estimated purity by LCMS analysis was 97.1%. Analysis condition A: Retention time=1.8 min; ESI-MS(+) m/z [M+3H]³⁺: 801.1.

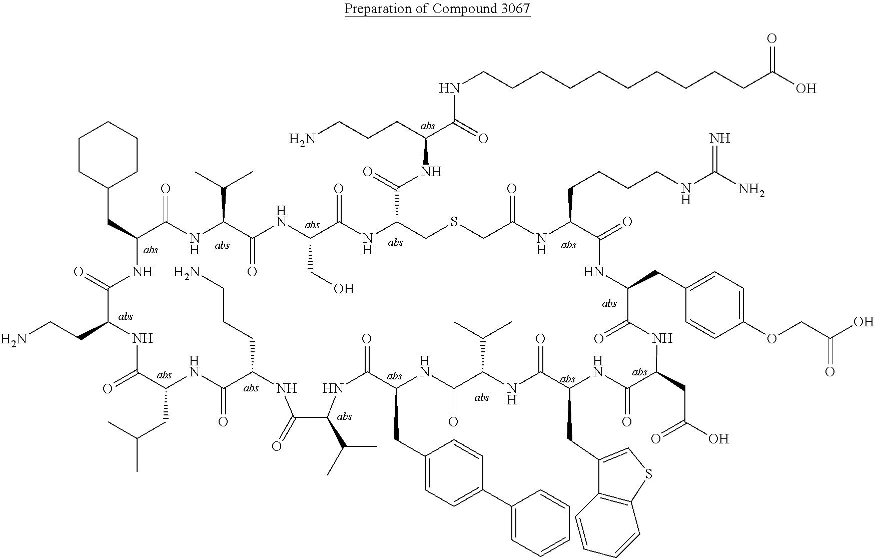 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2csc3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3067 was prepared on a 50 μmol scale. The yield of the product was 6 mg, and its estimated purity by LCMS analysis was 97.1%. Analysis condition B: Retention time=1.61 min; ESI-MS(+) m/z [M+2H]²⁺: 1129.1.

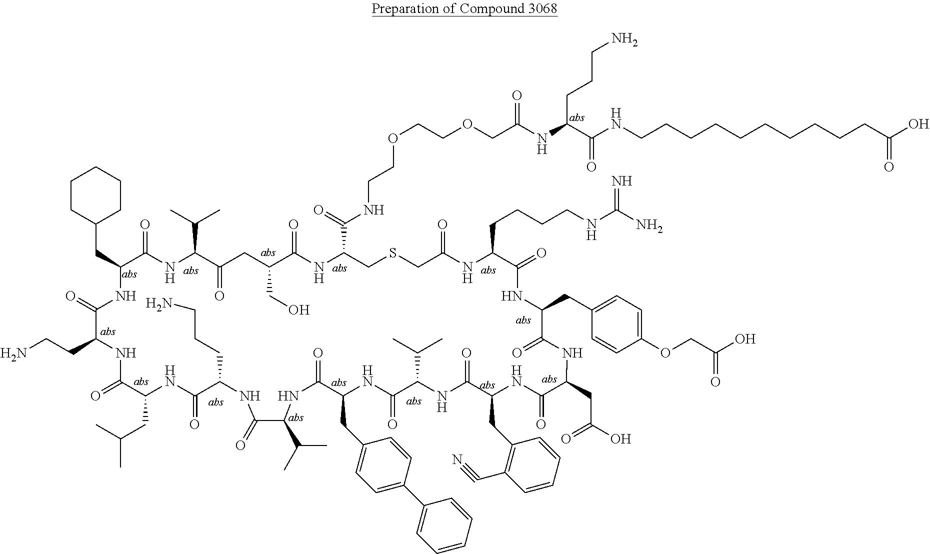 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccccc2C#N)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCN)C(=O)NCCCCCCCCCCC(=O)O)NC(=O)[C@H](CO)CC(=O)[C@H](C(C)C)NC(=O)[C@H](CC2CCCCC2)NC(=O)[C@H](CCN)NC1=O]

Compound 3068 was prepared on a 50 μmol scale. The yield of the product was 24.2 mg, and its estimated purity by LCMS analysis was 99%. Analysis condition B: Retention time=1.47 min; ESI-MS(+) m/z [M+3H]³⁺: 791.2.

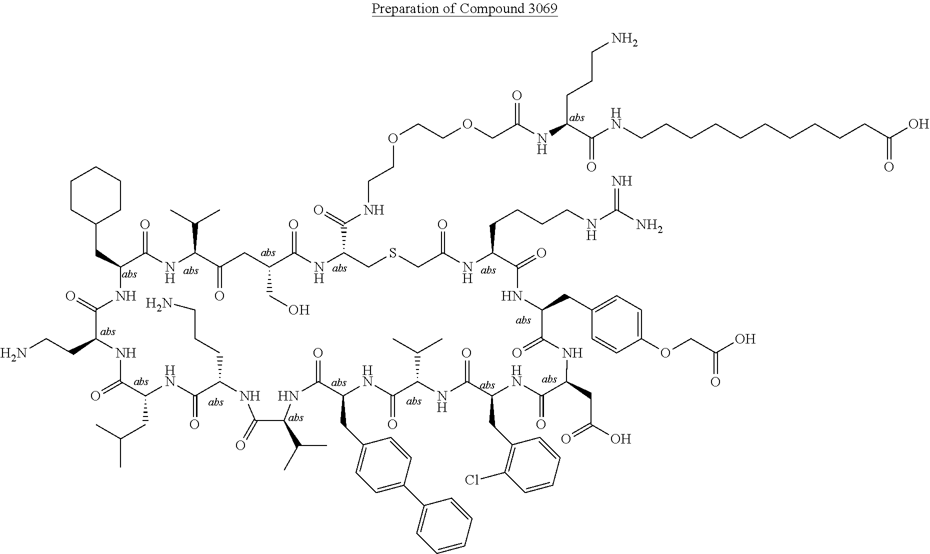 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccccc2Cl)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCN)C(=O)NCCCCCCCCCCC(=O)O)NC(=O)[C@H](CO)CC(=O)[C@H](C(C)C)NC(=O)[C@H](CC2CCCCC2)NC(=O)[C@H](CCN)NC1=O]

Compound 3069 was prepared on a 50 μmol scale. The yield of the product was 6.6 mg, and its estimated purity by LCMS analysis was 95.1%. Analysis condition B: Retention time=1.71 min; ESI-MS(+) m/z [M+3H]³⁺: 794.

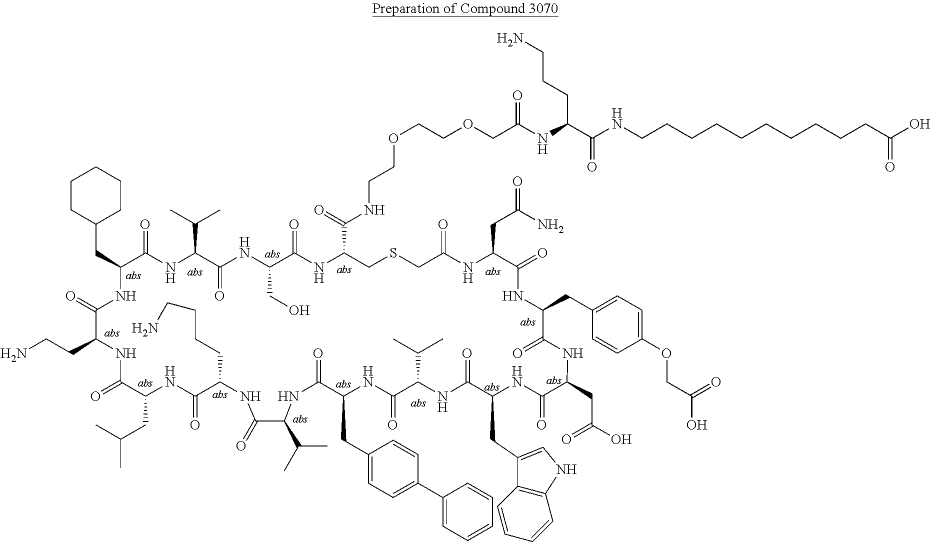 [SMILES: CC(C)C[C@H]1NC(=O)[C@H](CCCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CC(N)=O)NC(=O)CSC[C@@H](C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3070 was prepared on a 50 μmol scale. The yield of the product was 37.5 mg, and its estimated purity by LCMS analysis was 96.1%. Analysis condition B: Retention time=1.66 min; ESI-MS(+) m/z [M+2H]²⁺: 1171.8.

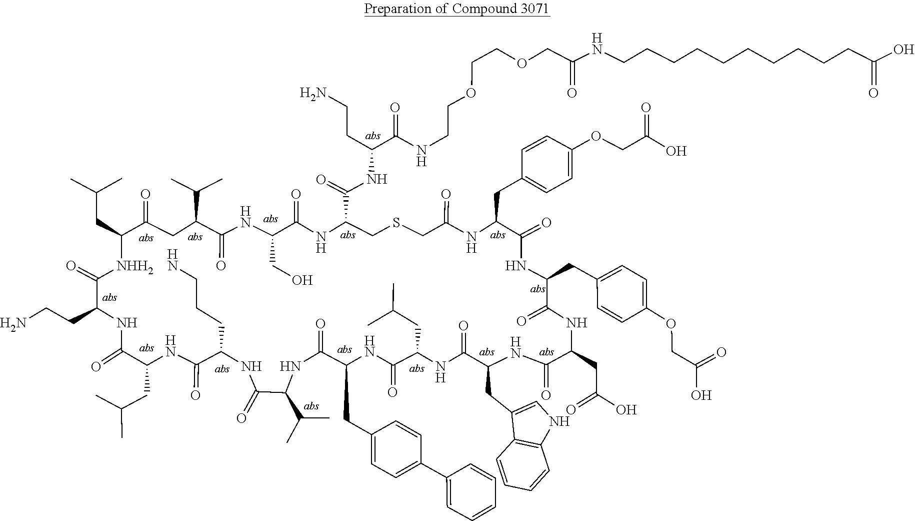

Compound 3071 was prepared on a 50 μmol scale. The yield of the product was 7.6 mg, and its estimated purity by LCMS analysis was 92.2%. Analysis condition B: Retention time=1.64 min; ESI-MS(+) m/z [M+2H]²⁺: 1199.

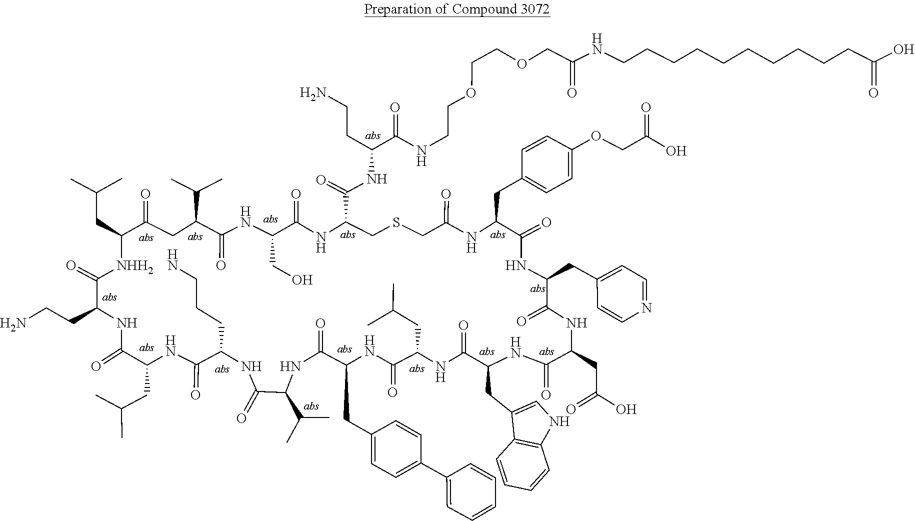

Compound 3072 was prepared on a 50 μmol scale. The yield of the product was 23.7 mg, and its estimated purity by LCMS analysis was 100%. Analysis condition A: Retention time=1.66 min; ESI-MS(+) m/z [M+2H]²⁺: 1162.3.

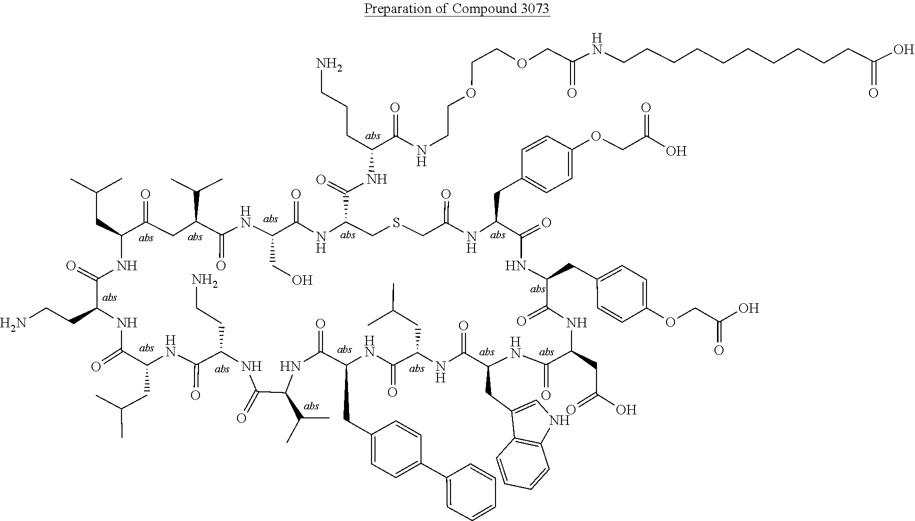 [SMILES: CC(C)C[C@@H]1NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@H](CCCN)C(=O)NCCOCCOCC(=O)NCCCCCCCCCCC(=O)O)NC(=O)[C@H](CO)NC(=O)[C@H](C(C)C)CC1=O]

Compound 3073 was prepared on a 50 μmol scale. The yield of the product was 7.1 mg, and its estimated purity by LCMS analysis was 95.9%. Analysis condition B: Retention time=1.46 min; ESI-MS(+) m/z [M+3H]³⁺: 799.2.

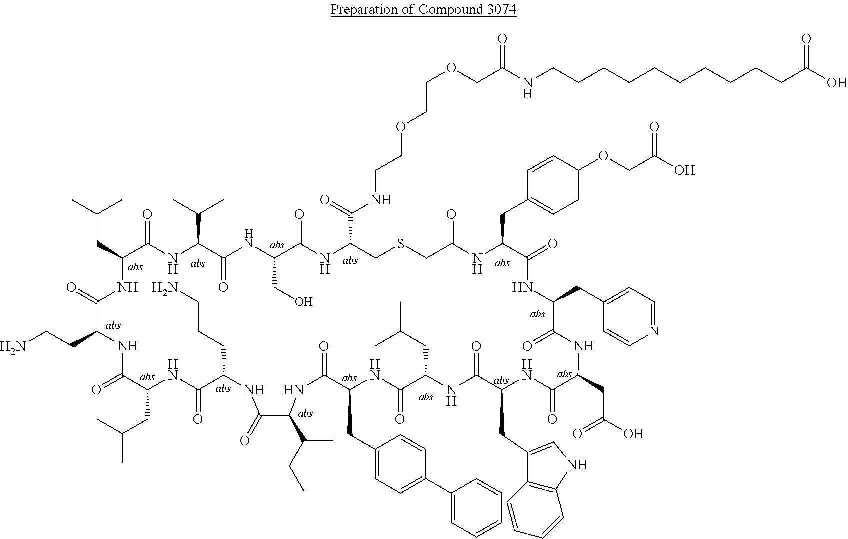 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 3074 was prepared on a 50 μmol scale. The yield of the product was 9.3 mg, and its estimated purity by LCMS analysis was 95.2%. Analysis condition A: Retention time=1.83 min; ESI-MS(+) m/z [M+2H]²⁺: 1104.9.

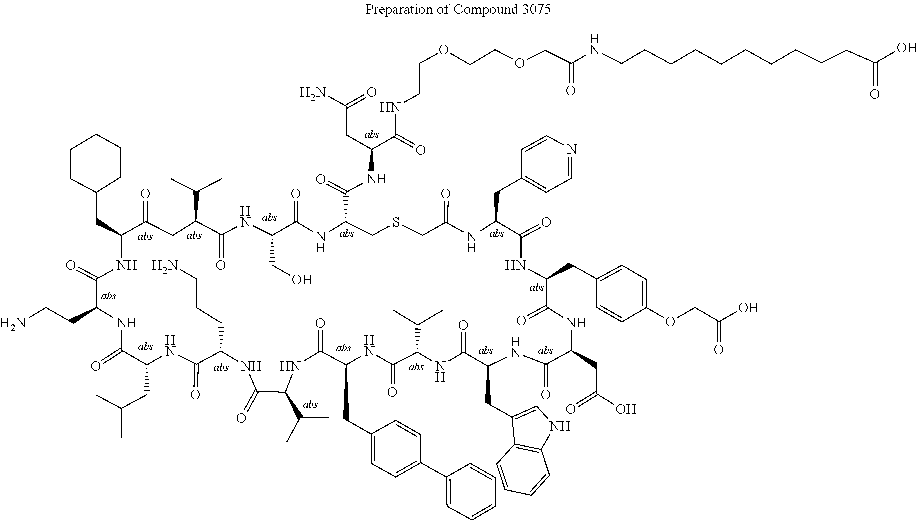 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CC(N)=O)C(=O)NCCOCCOCC(=O)NCCCCCCCCCCC(=O)O)NC(=O)[C@H](CO)NC(=O)[C@H](C(C)C)CC(=O)[C@H](CC2CCCCC2)NC(=O)[C@H](CCN)NC1=O]

Compound 3075 was prepared on a 50 μmol scale. The yield of the product was 6.5 mg, and its estimated purity by LCMS analysis was 97.3%. Analysis condition B: Retention time=1.61 min; ESI-MS(+) m/z [M+2H]²⁺: 1189.1.

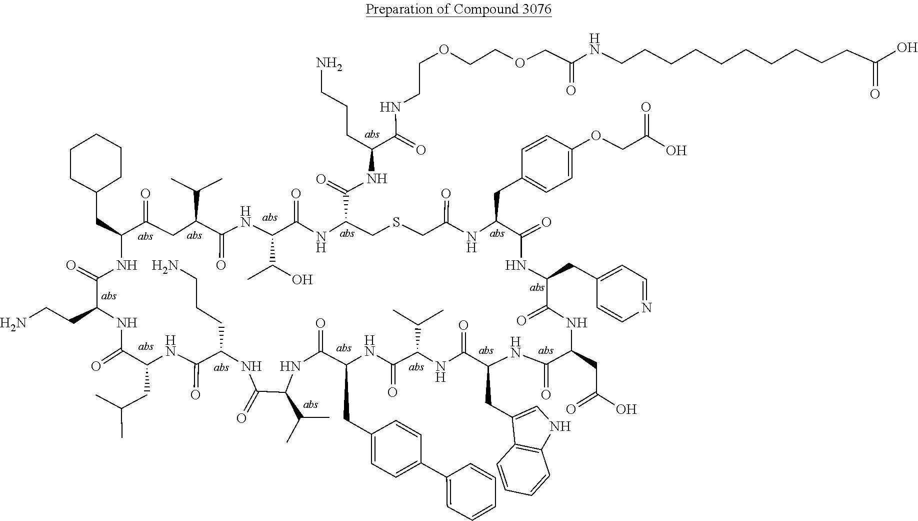 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](CCCN)C(=O)NCCOCCOCC(=O)NCCCCCCCCCCC(=O)O)NC(=O)[C@H](C(C)O)NC(=O)[C@H](C(C)C)CC(=O)[C@H](CC2CCCCC2)NC(=O)[C@H](CCN)NC1=O]

Compound 3076 was prepared on a 50 μmol scale. The yield of the product was 8.8 mg, and its estimated purity by LCMS analysis was 100%. Analysis condition A: Retention time=1.87 min; ESI-MS(+) m/z [M+2H]²⁺: 1189.1.

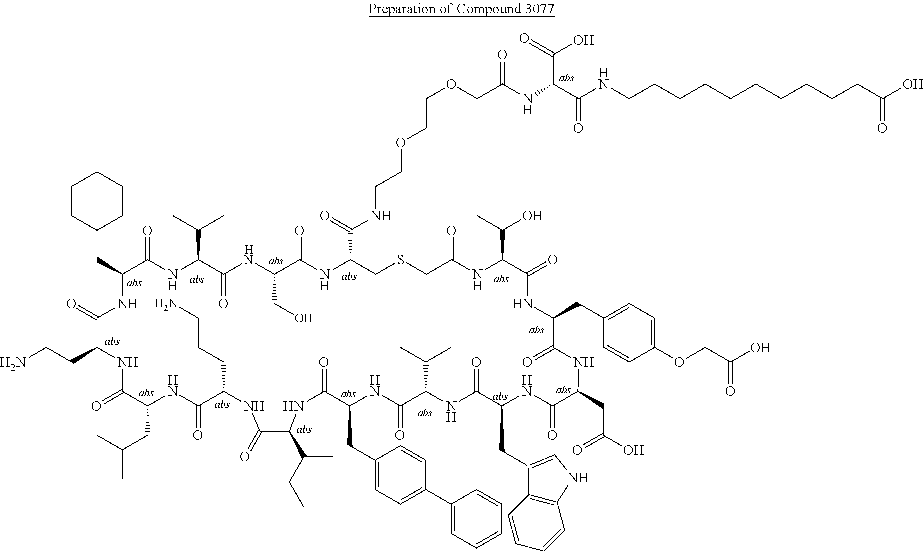

Compound 3077 was prepared on a 50 μmol scale. The yield of the product was 44 mg, and its estimated purity by LCMS analysis was 97%. Analysis condition A: Retention time=1.65 min; ESI-MS(+) m/z [M+2H]²⁺: 1173.1.

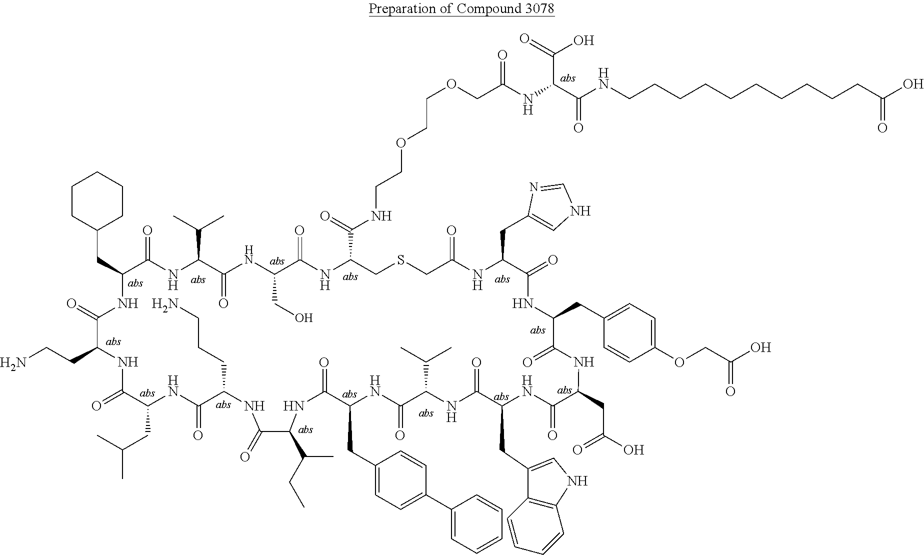 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2c[nH]cn2)NC(=O)CSC[C@@H](C(=O)NCCOCCOCC(=O)N[C@H](C(=O)O)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3078 was prepared on a 50 μmol scale. The yield of the product was 31 mg, and its estimated purity by LCMS analysis was 94.7%. Analysis condition B: Retention time=1.61 min; ESI-MS(+) m/z [M+2H]²⁺: 1191.4.

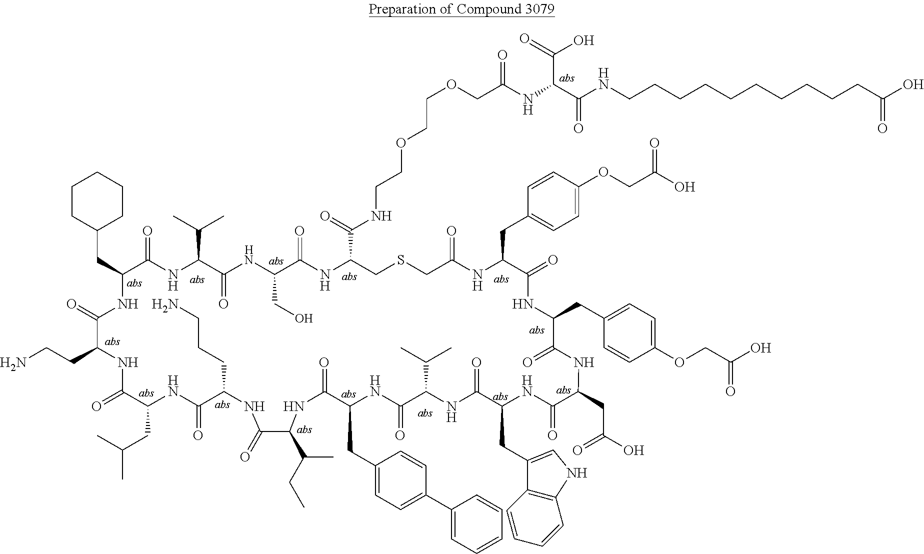 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@H](C(=O)O)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3079 was prepared on a 50 μmol scale. The yield of the product was 55.9 mg, and its estimated purity by LCMS analysis was 96.7%. Analysis condition A: Retention time=1.63 min; ESI-MS(+) m/z [M+2H]²⁺: 1233.1.

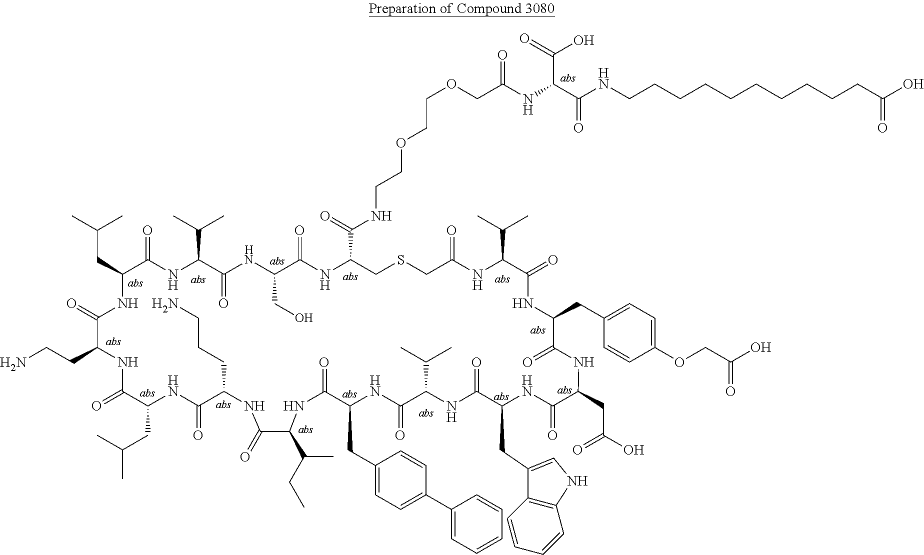 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)C)NC(=O)CSC[C@@H](C(=O)NCCOCCOCC(=O)N[C@H](C(=O)O)C(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 3080 was prepared on a 50 μmol scale. The yield of the product was 38.5 mg, and its estimated purity by LCMS analysis was 94.1%. Analysis condition A: Retention time=1.58 min; ESI-MS(+) m/z [M+2H]²⁺: 1153.1.

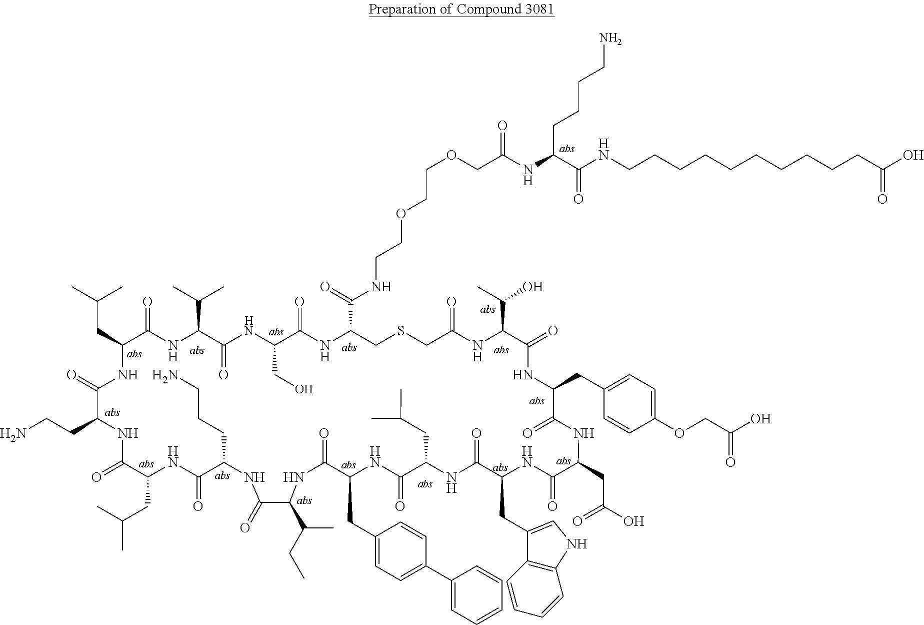 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@H](C)O)NC(=O)CSC[C@@H](C(=O)NCCOCCOCC(=O)N[C@@H](CCCCN)C(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 3081 was prepared on a 50 μmol scale. The yield of the product was 16.4 mg, and its estimated purity by LCMS analysis was 100%. Analysis condition A: Retention time=1.69 min; ESI-MS(+) m/z [M+2H]²⁺: 1159.

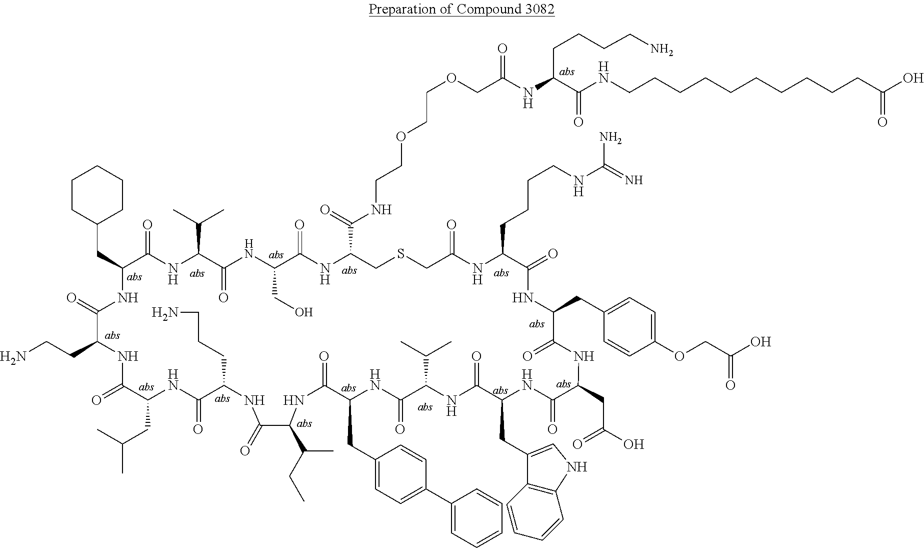 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3082 was prepared on a 50 μmol scale. The yield of the product was 18 mg, and its estimated purity by LCMS analysis was 97.7%. Analysis condition A: Retention time=1.46 min; ESI-MS(+) m/z [M+3H]³⁺: 805.

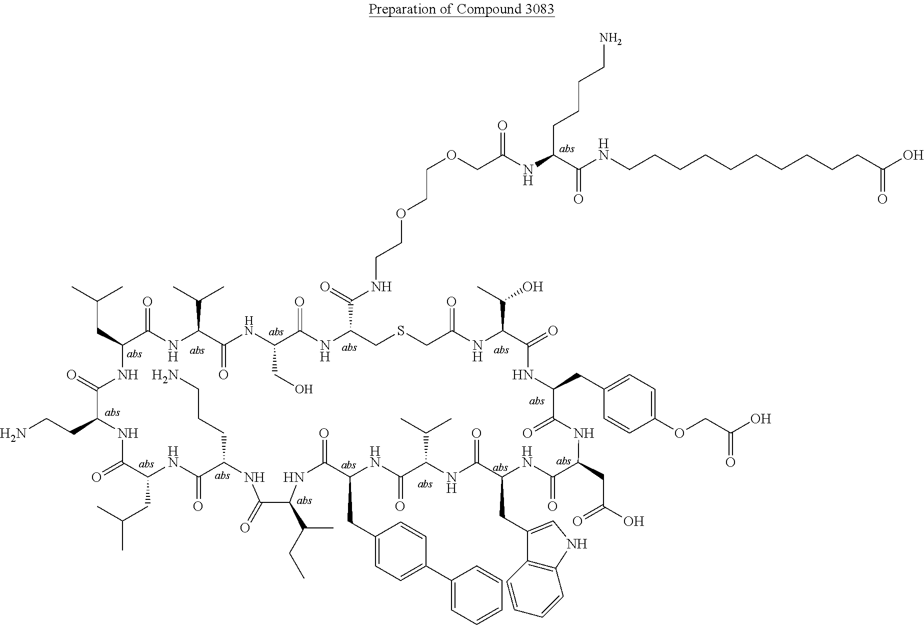 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H]([C@H](C)O)NC(=O)CSC[C@@H](C(=O)NCCOCCOCC(=O)N[C@@H](CCCCN)C(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 3083 was prepared on a 50 μmol scale. The yield of the product was 21.3 mg, and its estimated purity by LCMS analysis was 100%. Analysis condition A: Retention time=1.62 min; ESI-MS(+) m/z [M+3H]³⁺: 768.8.

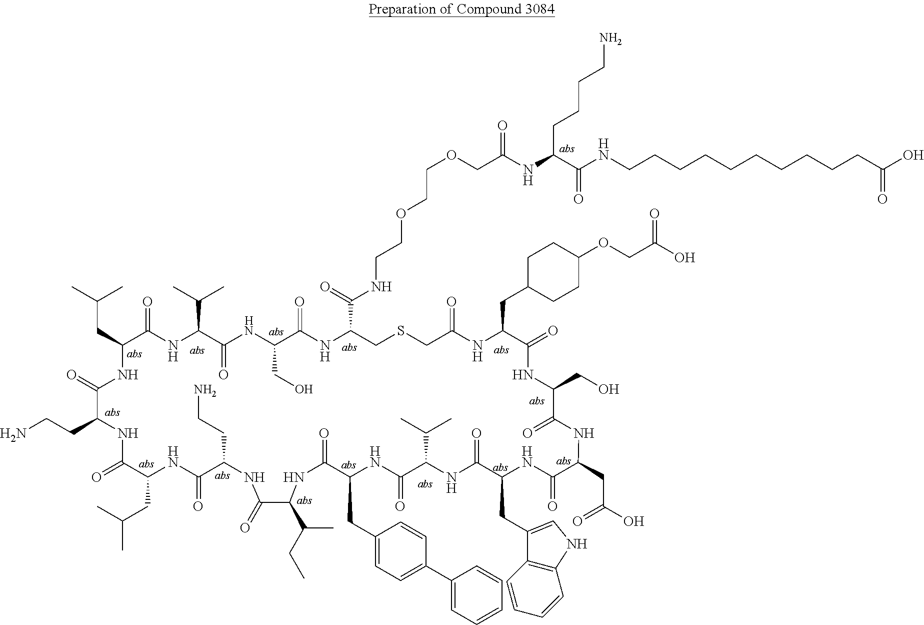 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CO)NC(=O)[C@H](CC2CCC(OCC(=O)O)CC2)NC(=O)CSC[C@@H](C(=O)NCCOCCOCC(=O)N[C@@H](CCCCN)C(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCN)NC1=O]

Compound 3084 was prepared on a 50 μmol scale. The yield of the product was 34.5 mg, and its estimated purity by LCMS analysis was 92.3%. Analysis condition B: Retention time=1.49 min; ESI-MS(+) m/z [M+2H]²⁺: 759.1.

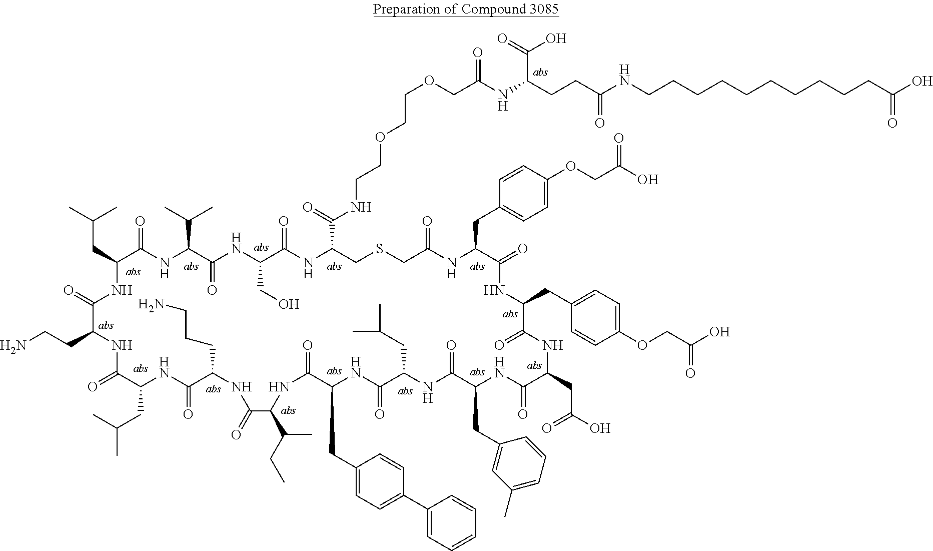 [SMILES: CCC(C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)N[C@@H](CCC(=O)NCCCCCCCCCCC(=O)O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 3085 was prepared on a 50 μmol scale. The yield of the product was 19.6 mg, and its estimated purity by LCMS analysis was 97.3%. Analysis condition B: Retention time=1.77 min; ESI-MS(+) m/z [M+3H]³⁺: 805.2.

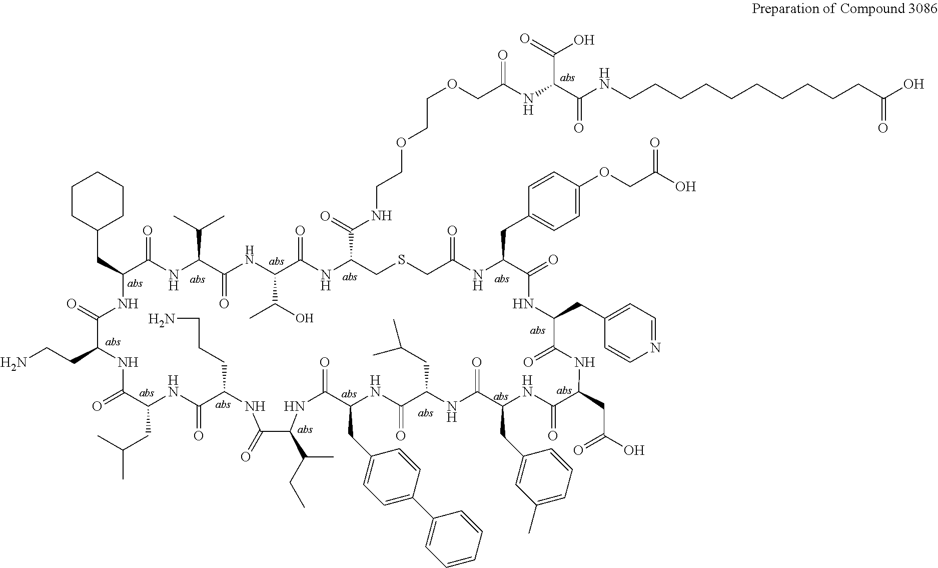 [SMILES: CCC(C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCCOCCOCC(=O)N[C@H](C(=O)O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3086 was prepared on a 50 μmol scale. The yield of the product was 70.3 mg, and its estimated purity by LCMS analysis was 94.8%. Analysis condition B: Retention time-1.66 min; ESI-MS(+) m/z [M+3H]³⁺: 799.4.

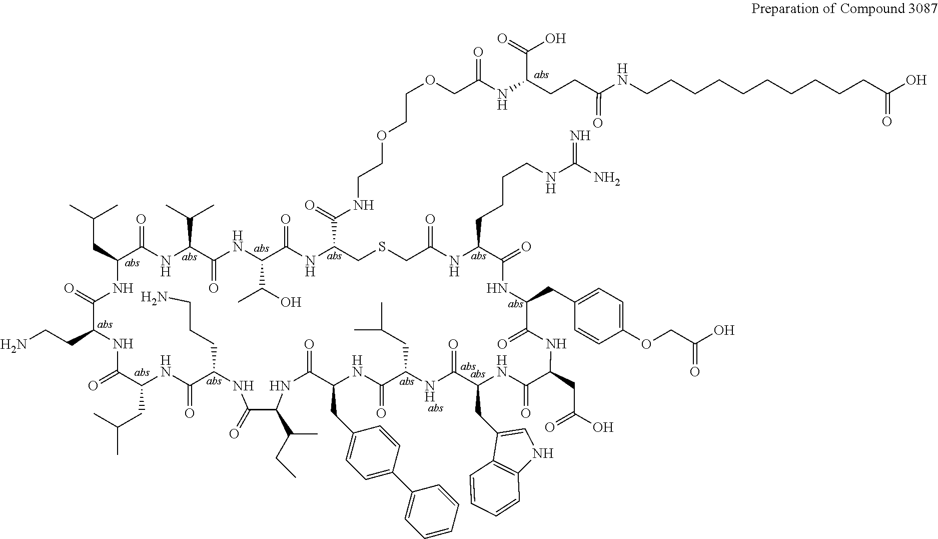 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCC(=O)NCCCCCCCCCCC(=O)O)C(=O)O)NC(=O)[C@H](C(C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 3087 was prepared on a 50 μmol scale. The yield of the product was 40.4 mg, and its estimated purity by LCMS analysis was 95.9%. Analysis condition B: Retention time=1.66 min; ESI-MS(+) m/z [M+2H]²⁺: 815.0.

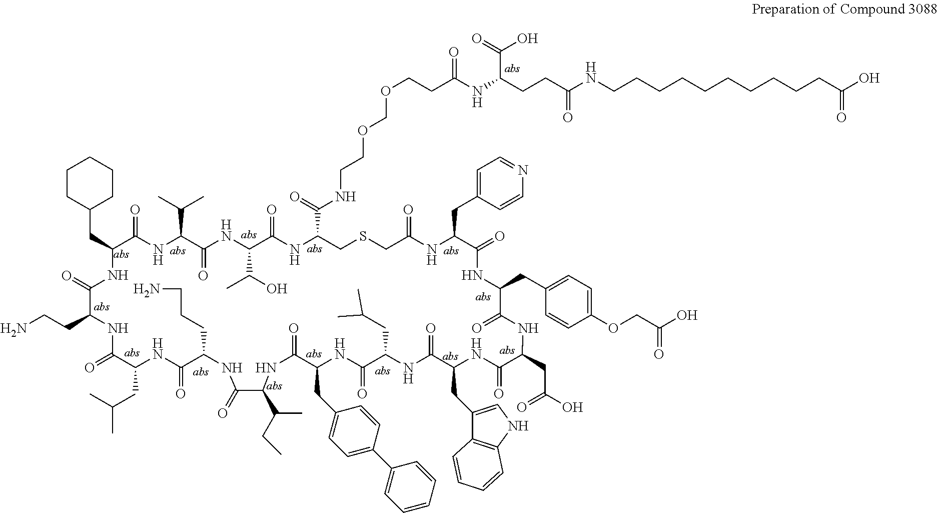 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCCOCOCCC(=O)N[C@@H](CCC(=O)NCCCCCCCCCCC(=O)O)C(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3088 was prepared on a 50 μmol scale. The yield of the product was 41.5 mg, and its estimated purity by LCMS analysis was 95.2%. Analysis condition A: Retention time=1.71 min; ESI-MS(+) m/z [M+2H]²⁺: 1210.2.

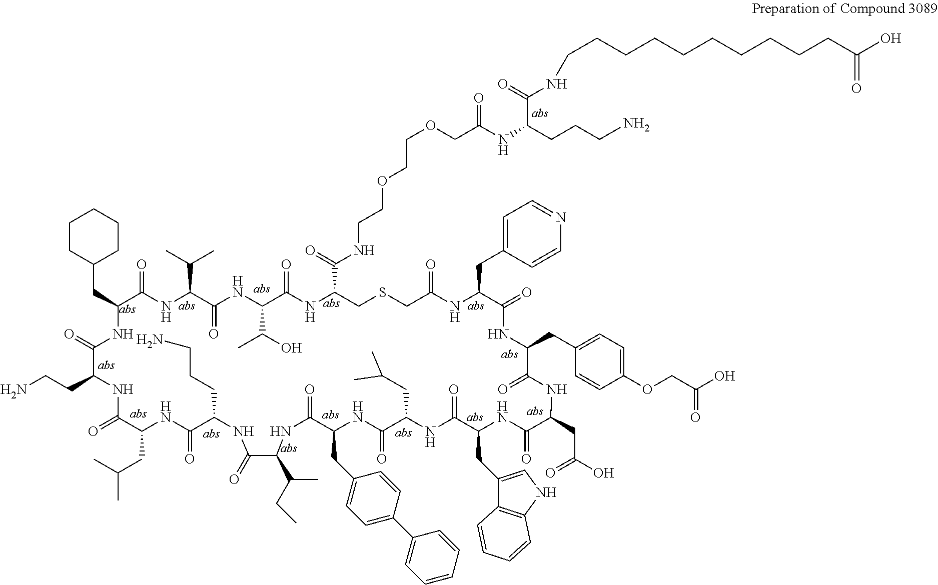

Compound 3089 was prepared on a 50 μmol scale. The yield of the product was 33 mg, and its estimated purity by LCMS analysis was 95.3%. Analysis condition A: Retention time=1.73 min; ESI-MS(+) m/z [M+2H]²⁺: 1202.6.

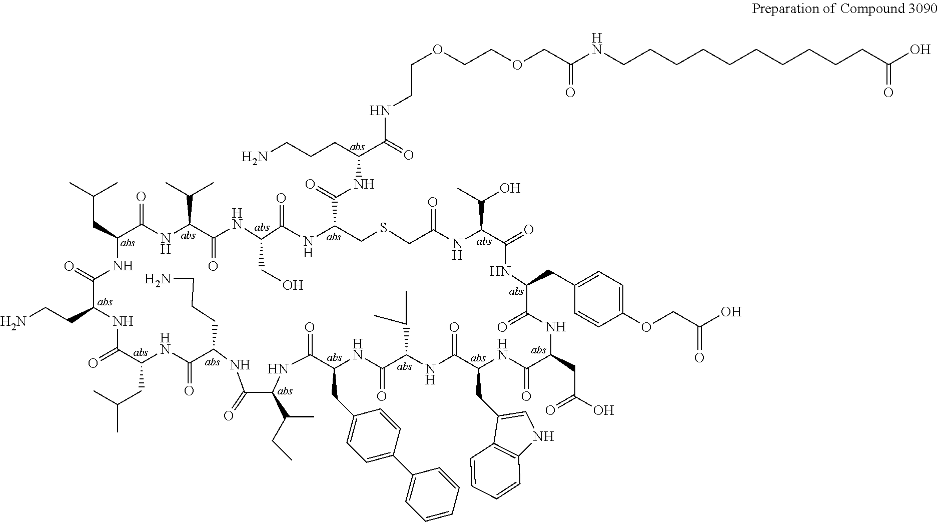 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CCCN)C(=O)NCCOCCOCC(=O)NCCCCCCCCCCC(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 3090 was prepared on a 50 μmol scale. The yield of the product was 13.9 mg, and its estimated purity by LCMS analysis was 97.4%. Analysis condition B: Retention time=1.47 min; ESI-MS(+) m/z [M+3H]³⁺: 764.1.

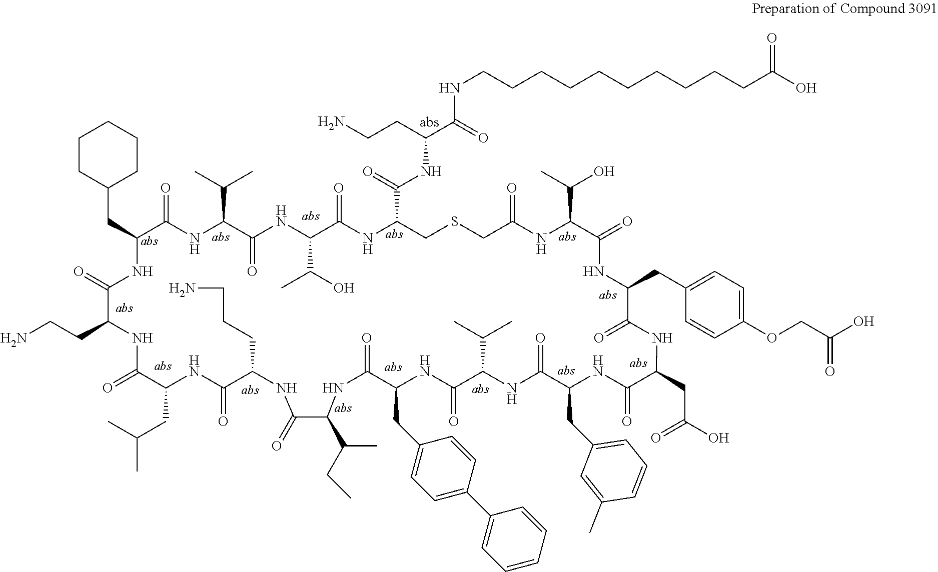 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3091 was prepared on a 50 μmol scale. The yield of the product was 15 mg, and its estimated purity by LCMS analysis was 96.8%. Analysis condition B: Retention time=1.77 min; ESI-MS(+) m/z [M+2H]²⁺: 1080.

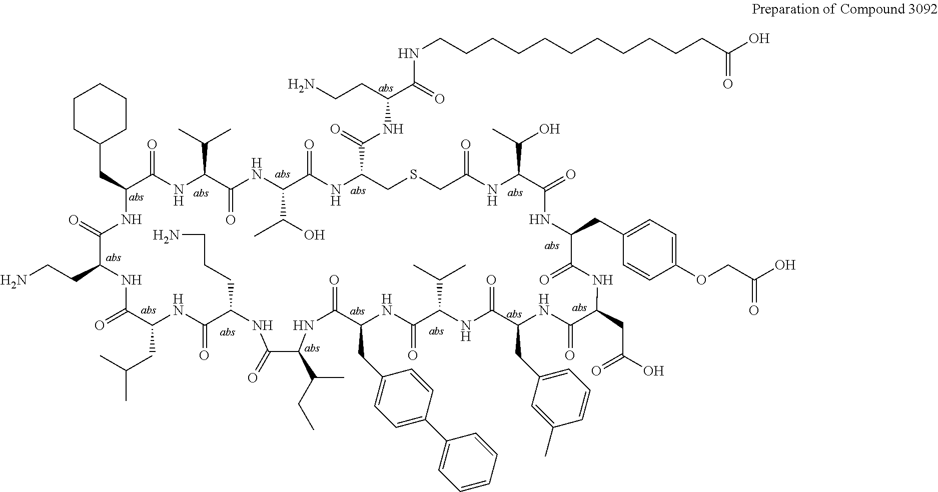 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CCN)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3092 was prepared on a 50 μmol scale. The yield of the product was 4.8 mg, and its estimated purity by LCMS analysis was 95%. Analysis condition A: Retention time=1.66 min; ESI-MS(+) m/z [M+2H]²⁺: 1087.3.

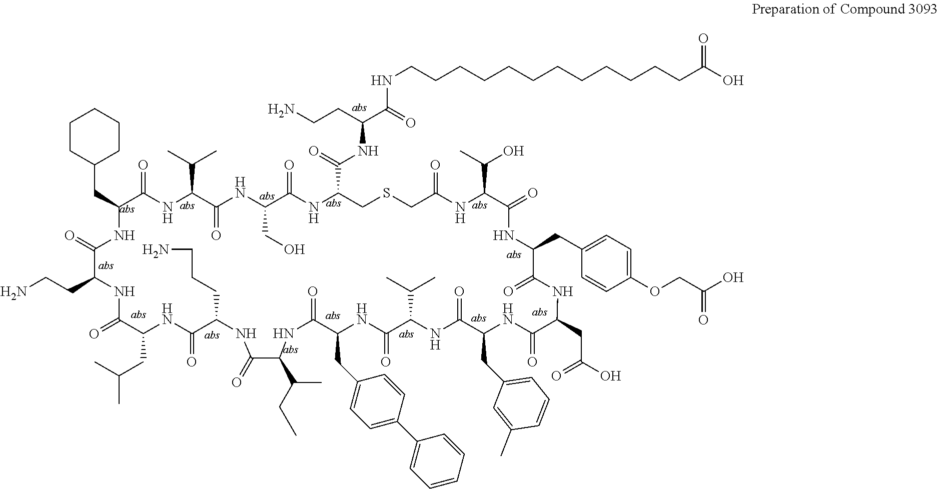 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3093 was prepared on a 50 μmol scale. The yield of the product was 3.7 mg, and its estimated purity by LCMS analysis was 95.1%. Analysis condition A: Retention time=1.65 min; ESI-MS(+) m/z [M+2H]²⁺: 1087.4.

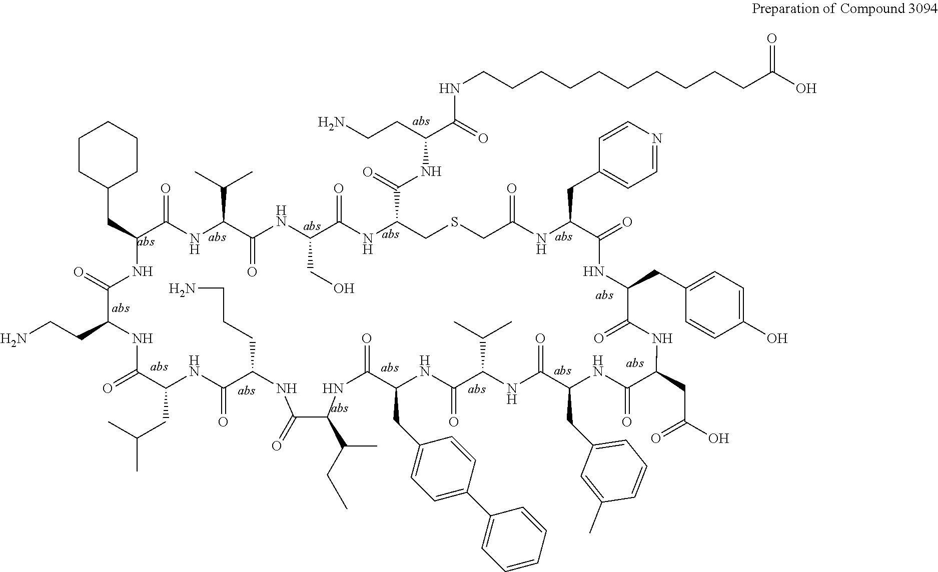 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@H](CCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3094 was prepared on a 50 μmol scale. The yield of the product was 6.1 mg, and its estimated purity by LCMS analysis was 95.7%. Analysis condition B: Retention time=1.58 min; ESI-MS(+) m/z [M+2H]²⁺: 1075.2.

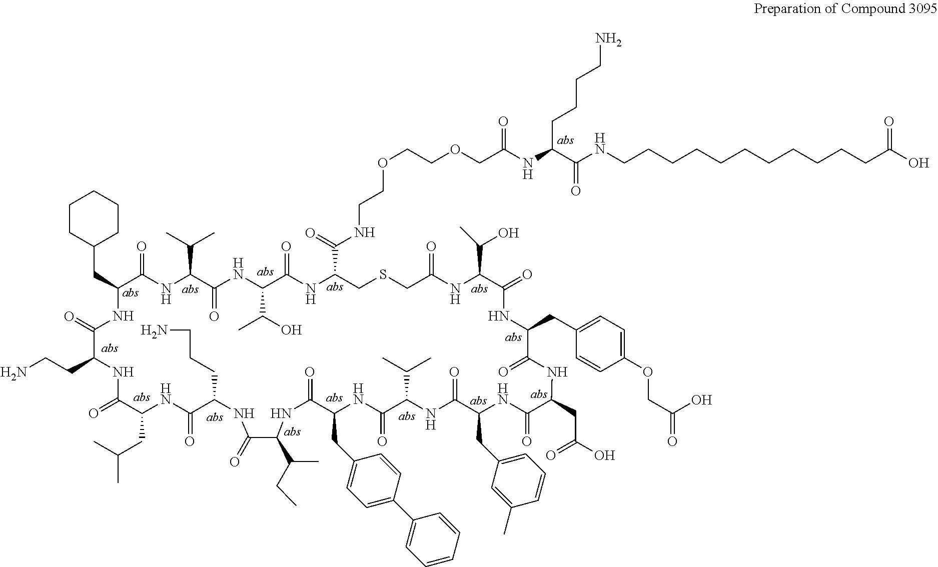 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)N[C@@H](CCCCN)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3095 was prepared on a 50 μmol scale. The yield of the product was 19.5 mg, and its estimated purity by LCMS analysis was 95%. Analysis condition B: Retention time=1.72 min; ESI-MS(+) m/z [M+3H]³⁺: 783.4.

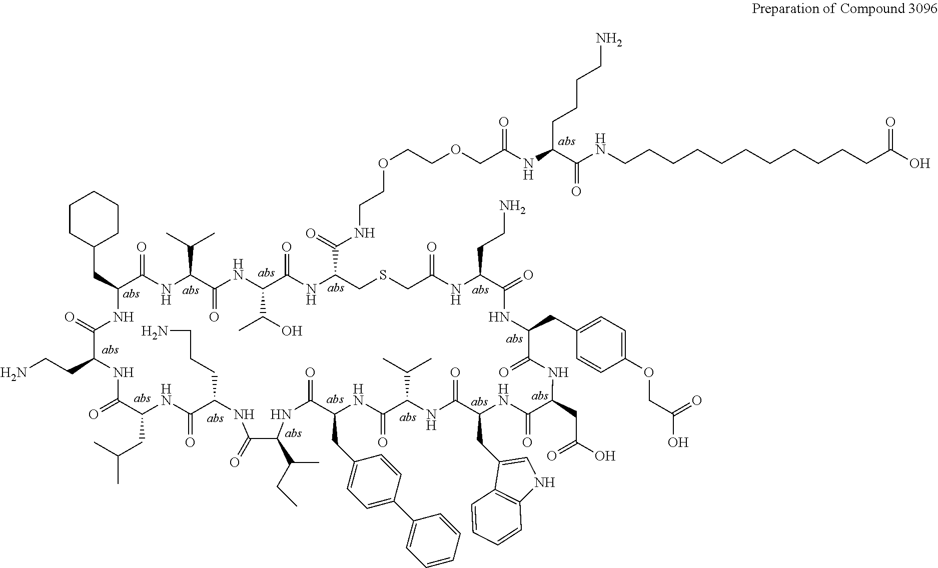 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)NC(=O)CSC[C@@H](C(=O)NCCOCCOCC(=O)N[C@@H](CCCCN)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3096 was prepared on a 50 μmol scale. The yield of the product was 28.6 mg, and its estimated purity by LCMS analysis was 100%. Analysis condition A: Retention time=1.5 min; ESI-MS(+) m/z [M+3H]³⁺: 791.2.

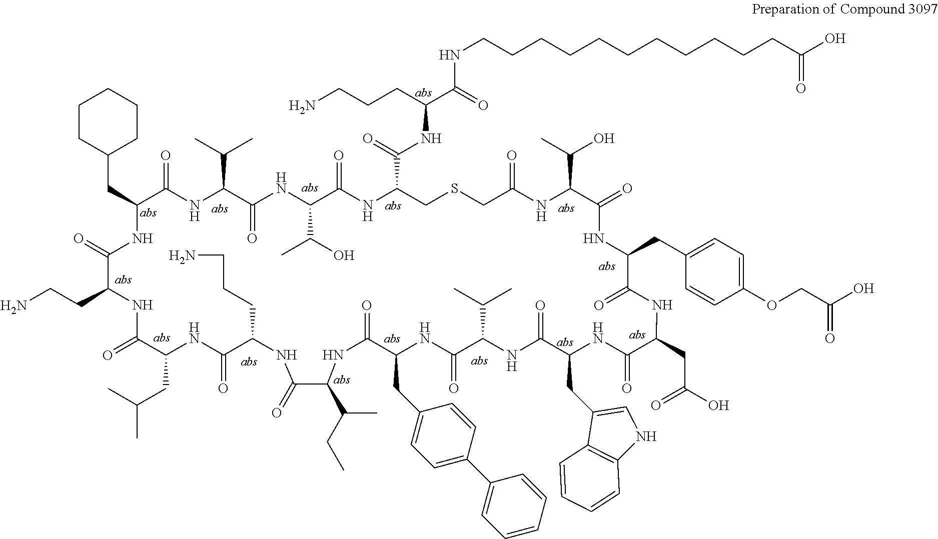 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3097 was prepared on a 50 μmol scale. The yield of the product was 7.1 mg, and its estimated purity by LCMS analysis was 96.7%. Analysis condition B: Retention time=1.65 min; ESI-MS(+) m/z [M+2H]²⁺: 1107.1.

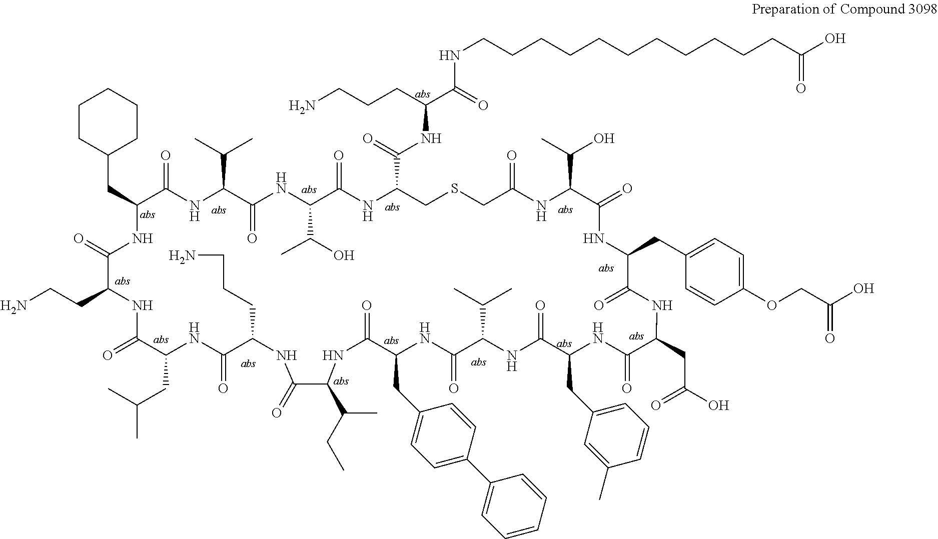 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCCN)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3098 was prepared on a 50 μmol scale. The yield of the product was 3.7 mg, and its estimated purity by LCMS analysis was 96.4%. Analysis condition B: Retention time-1.75 min; ESI-MS(+) m/z [M+3H]³⁺: 730.

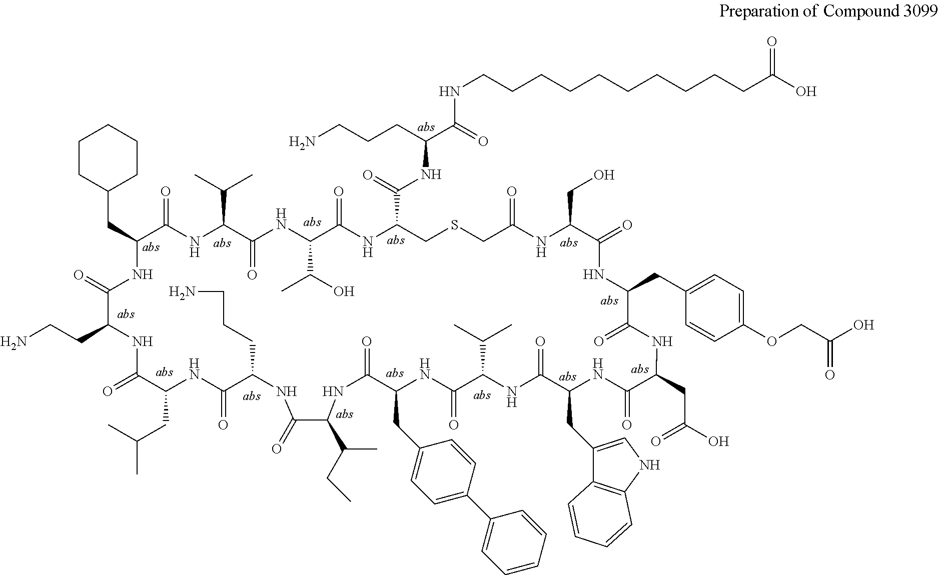 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CO)NC(=O)CSC[C@@H](C(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3099 was prepared on a 50 μmol scale. The yield of the product was 13.8 mg, and its estimated purity by LCMS analysis was 100%. Analysis condition A: Retention time-1.75 min; ESI-MS(+) m/z [M+2H]²⁺: 1092.8.

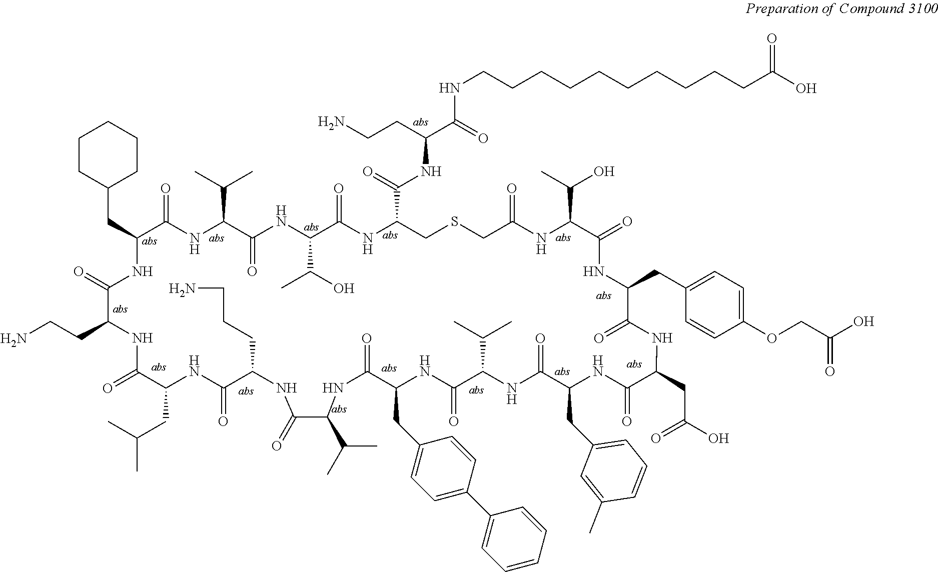 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3100 was prepared on a 50 μmol scale. The yield of the product was 5.4 mg, and its estimated purity by LCMS analysis was 100%. Analysis condition A: Retention time=1.77 min; ESI-MS(+) m/z [M+2H]²⁺: 1080.2.

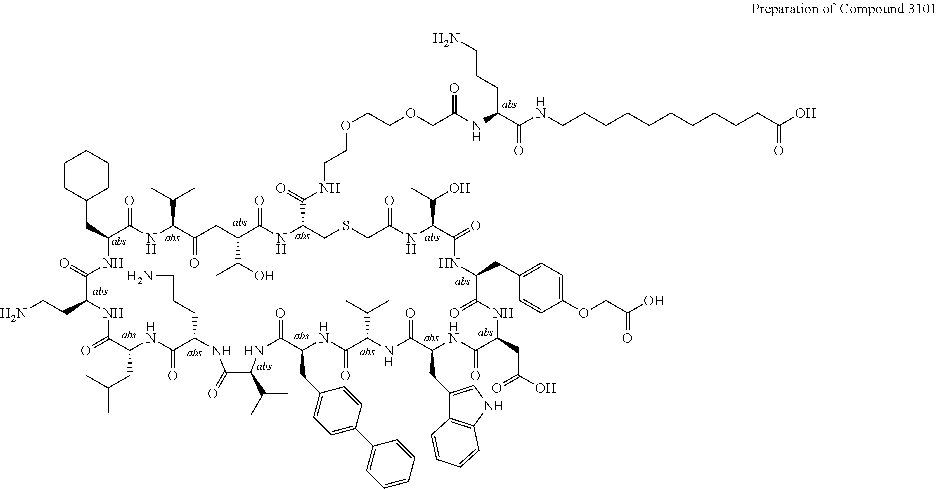 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)N[C@@H](CCCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CCN)NC1=O]

Compound 3101 was prepared on a 50 μmol scale. The yield of the product was 28.7 mg, and its estimated purity by LCMS analysis was 98.8%. Analysis condition A: Retention time=1.63 min; ESI-MS(+) m/z [M+2H]²⁺:

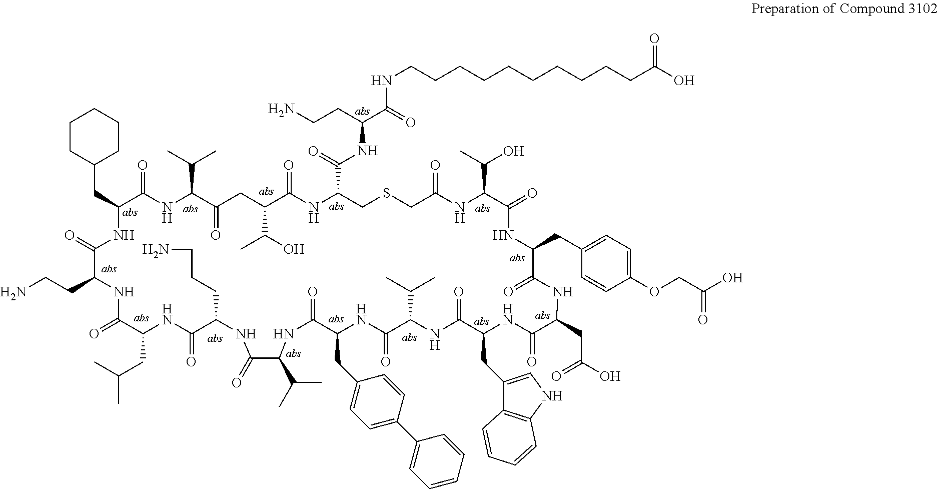 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CCN)NC1=O]

Compound 3102 was prepared on a 50 μmol scale. The yield of the product was 13.5 mg, and its estimated purity by LCMS analysis was 98.4%. Analysis condition B: Retention time=1.52 min; ESI-MS(+) m/z [M+2H]²⁺: 1086.3.

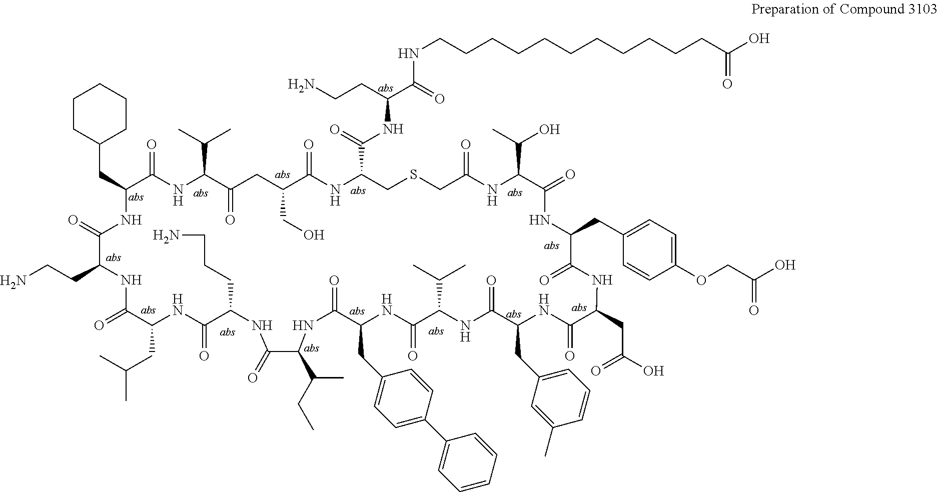 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3103 was prepared on a 25 μmol scale. The yield of the product was 3 mg, and its estimated purity by LCMS analysis was 100%. Analysis condition A: Retention time=1.77 min; ESI-MS(+) m/z [M+2H]²⁺: 1080.

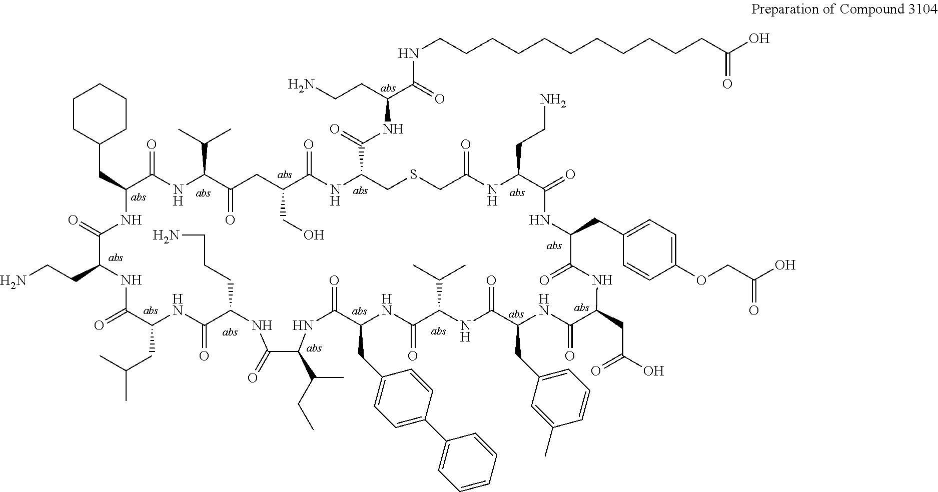 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)NCCCCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3104 was prepared on a 25 μmol scale. The yield of the product was 2.3 mg, and its estimated purity by LCMS analysis was 97.9%. Analysis condition A: Retention time=1.74 min; ESI-MS(+) m/z [M+2H]²⁺: 1080.2.

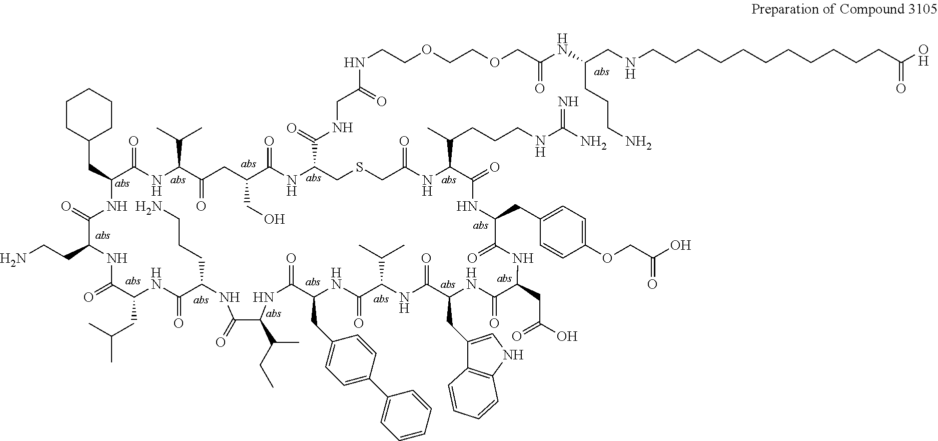 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)CCCNC(=N)N)NC(=O)CSC[C@@H](C(=O)NCC(=O)NCCOCCOCC(=O)N[C@@H](CCCN)CNCCCCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3105 was prepared on a 50 μmol scale. The yield of the product was 1.7 mg, and its estimated purity by LCMS analysis was 95.1%. Analysis condition B: Retention time=1.47 min; ESI-MS(+) m/z [M+3H]³⁺: 824.

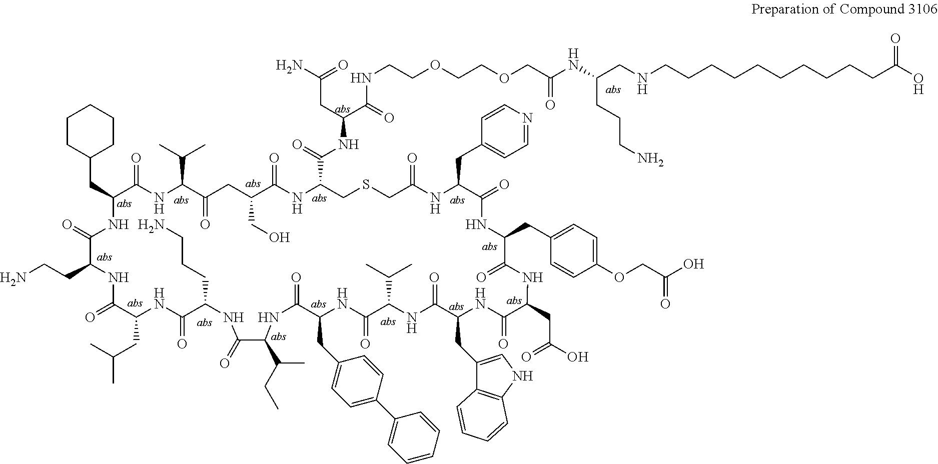 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CC(N)=O)C(=O)NCCOCCOCC(=O)N[C@@H](CCCN)CNCCCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3106 was prepared on a 50 μmol scale. The yield of the product was 3.5 mg, and its estimated purity by LCMS analysis was 96%. Analysis condition B: Retention time=1.46 min; ESI-MS(+) m/z [M+3H]³⁺: 831.

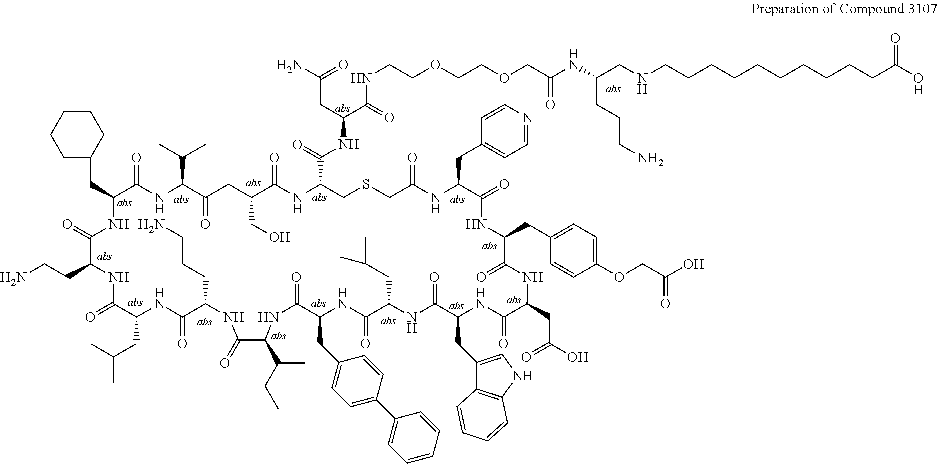 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CC(N)=O)C(=O)NCCOCCOCC(=O)N[C@@H](CCCN)CNCCCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3107 was prepared on a 50 μmol scale. The yield of the product was 7.4 mg, and its estimated purity by LCMS analysis was 97.5%. Analysis condition A: Retention time=1.61 min; ESI-MS(+) m/z [M+3H]³⁺: 836.2.

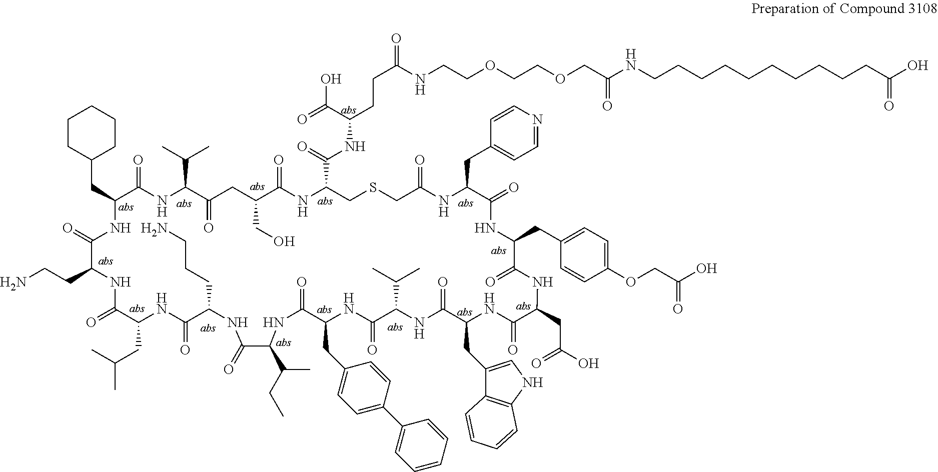 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCC(=O)NCCOCCOCC(=O)NCCCCCCCCCCC(=O)O)C(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3108 was prepared on a 50 μmol scale. The yield of the product was 3.9 mg, and its estimated purity by LCMS analysis was 97.9%. Analysis condition B: Retention time=1.54 min; ESI-MS(+) m/z [M+3H]³⁺: 788.1.

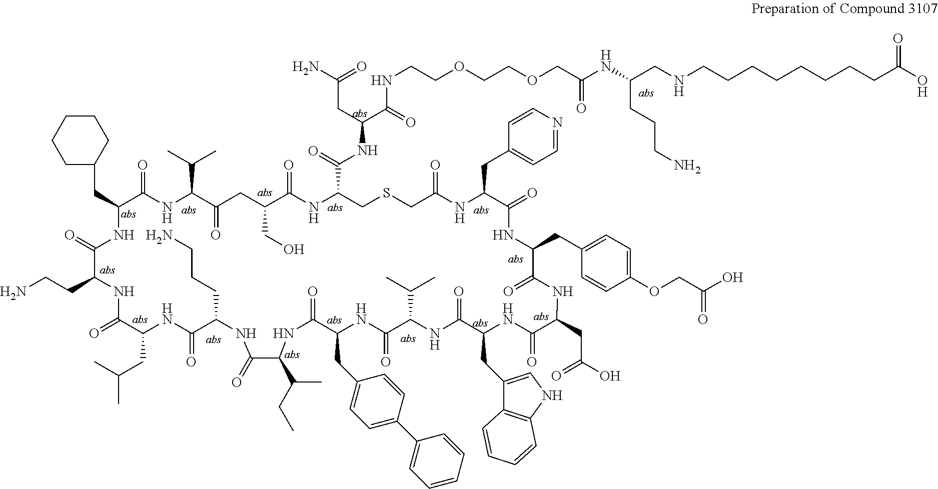 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CC(N)=O)C(=O)NCCOCCOCC(=O)N[C@@H](CCCN)CNCCCCCCCCC(=O)O)NC(=O)[C@H](CO)CC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3109 was prepared on a 50 μmol scale. The yield of the product was 5.4 mg, and its estimated purity by LCMS analysis was 96.9%. Analysis condition A: Retention time=1.68 min; ESI-MS(+) m/z [M+2H]²⁺: 1238.

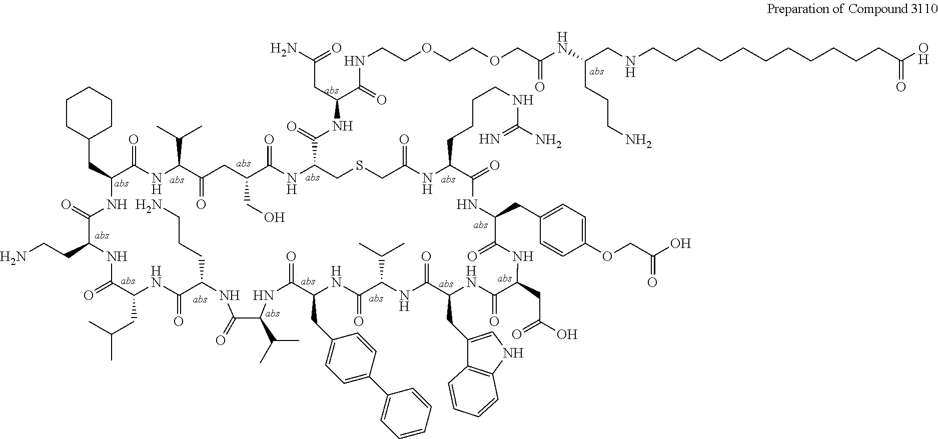 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CC(N)=O)C(=O)NCCOCCOCC(=O)N[C@@H](CCCN)CNCCCCCCCCCCCC(=O)O)NC(=O)[C@H](CO)CC(=O)[C@H](C(C)C)NC(=O)[C@H](CC2CCCCC2)NC(=O)[C@H](CCN)NC1=O]

Compound 3110 was prepared on a 50 μmol scale. The yield of the product was 3.9 mg, and its estimated purity by LCMS analysis was 95.2%. Analysis condition A: Retention time-1.46 min; ESI-MS(+) m/z [M+2H]²⁺: 1257.1.

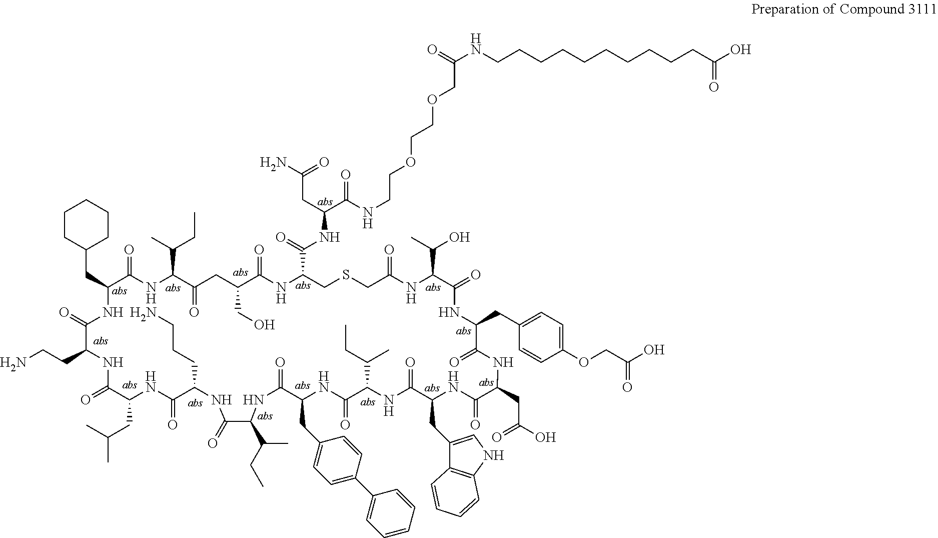 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)NCCOCCOCC(=O)NCCCCCCCCCCC(=O)O)NC(=O)[C@H](CO)CC1=O]

Compound 3111 was prepared on a 50 μmol scale. The yield of the product was 9.3 mg, and its estimated purity by LCMS analysis was 94.2%. Analysis condition A: Retention time=1.71 min; ESI-MS(+) m/z [M+2H]²⁺: 1179.9.

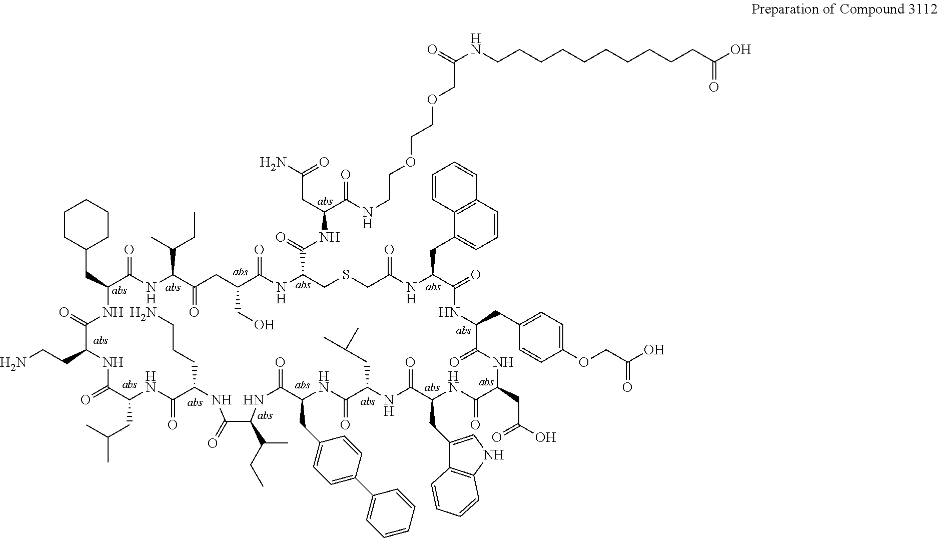 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cccc3ccccc23)NC(=O)CSC[C@@H](C(=O)N[C@@H](CC(N)=O)C(=O)NCCOCCOCC(=O)NCCCCCCCCCCC(=O)O)NC(=O)[C@H](CO)CC1=O]

Compound 3112 was prepared on a 50 μmol scale. The yield of the product was 14.5 mg, and its estimated purity by LCMS analysis was 100%. Analysis condition A: Retention time=2 min; ESI-MS(+) m/z [M+2H]²⁺: 1228.2.

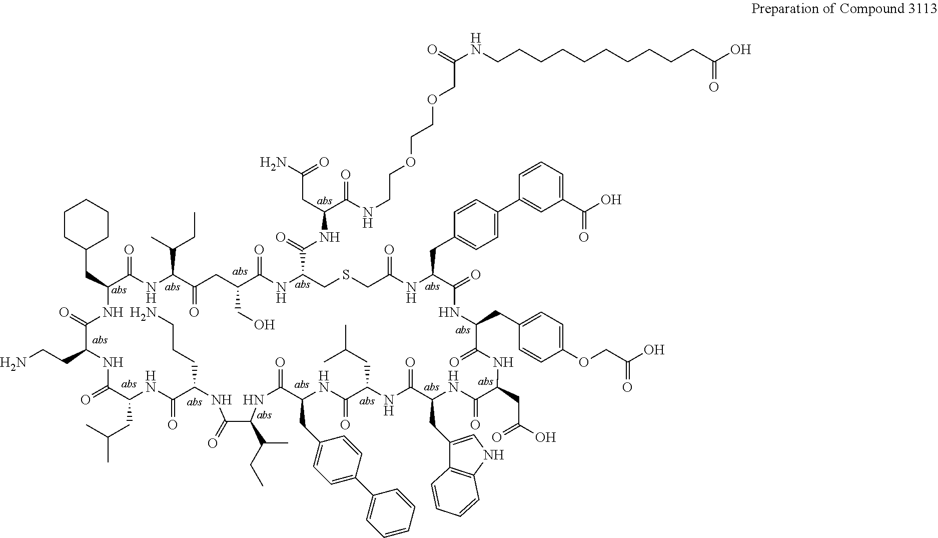 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(-c3cccc(C(=O)O)c3)cc2)NC(=O)CSC[C@@H](C(=O)N[C@@H](CC(N)=O)C(=O)NCCOCCOCC(=O)NCCCCCCCCCCC(=O)O)NC(=O)[C@H](CO)CC1=O]

Compound 3113 was prepared on a 50 μmol scale. The yield of the product was 21.9 mg, and its estimated purity by LCMS analysis was 95.9%. Analysis condition A: Retention time=1.67 min; ESI-MS(+) m/z [M+2H]²⁺: 1263.

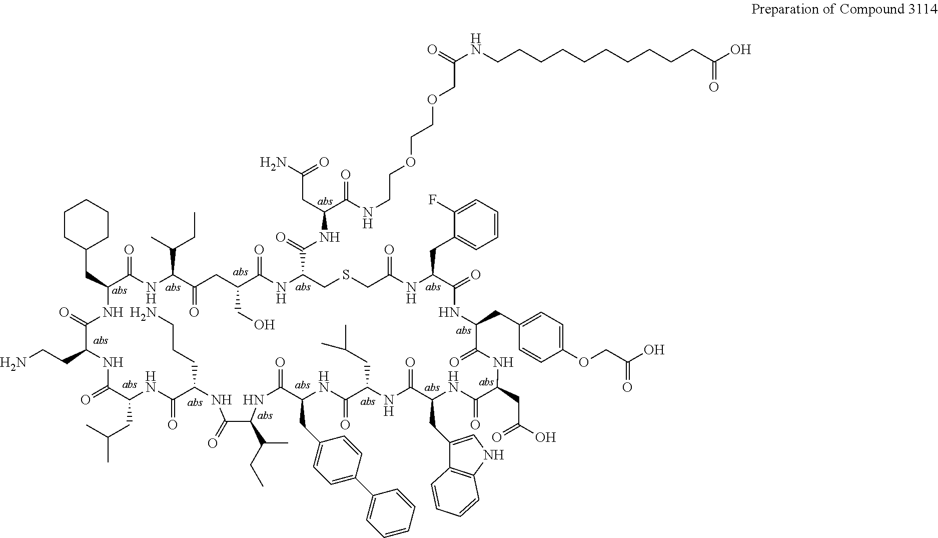 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2F)NC(=O)CSC[C@@H](C(=O)N[C@@H](CC(N)=O)C(=O)NCCOCCOCC(=O)NCCCCCCCCCCC(=O)O)NC(=O)[C@H](CO)CC1=O]

Compound 3114 was prepared on a 50 μmol scale. The yield of the product was 20.6 mg, and its estimated purity by LCMS analysis was 91.9%. Analysis condition A: Retention time=1.83 min; ESI-MS(+) m/z [M+2H]²⁺: 1211.2.

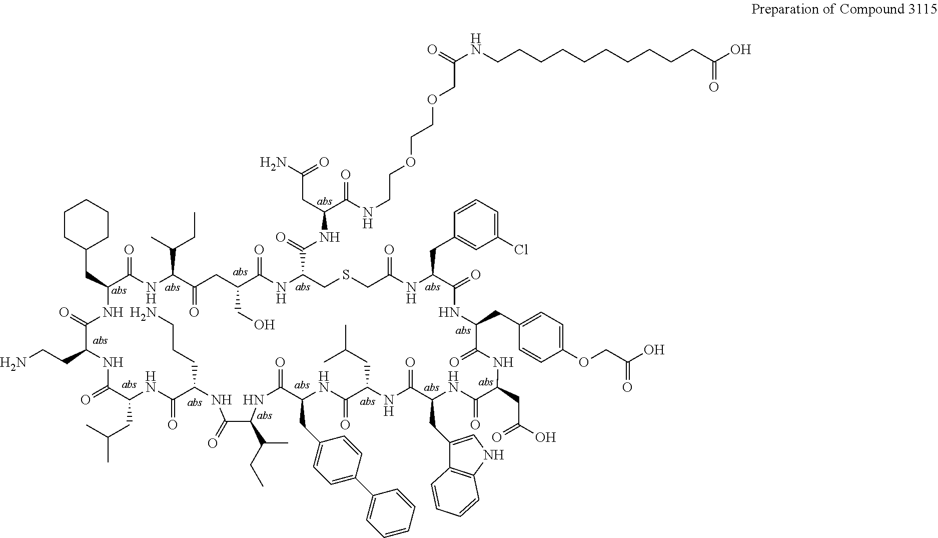 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cccc(Cl)c2)NC(=O)CSC[C@@H](C(=O)N[C@@H](CC(N)=O)C(=O)NCCOCCOCC(=O)NCCCCCCCCCCC(=O)O)NC(=O)[C@H](CO)CC1=O]

Compound 3115 was prepared on a 50 μmol scale. The yield of the product was 16.2 mg, and its estimated purity by LCMS analysis was 92.5%. Analysis condition B: Retention time-1.97 min; ESI-MS(+) m/z [M+2H]²⁺: 1220.2.

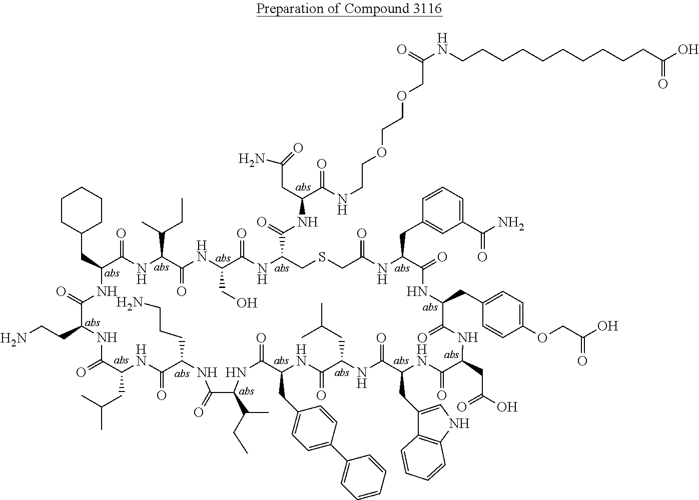 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cccc(C(N)=O)c2)NC(=O)CSC[C@@H](C(=O)N[C@@H](CC(N)=O)C(=O)NCCOCCOCC(=O)NCCCCCCCCCCC(=O)O)NC(=O)[C@H](CO)NC1=O]

Compound 3116 was prepared on a 50 μmol scale. The yield of the product was 16.8 mg, and its estimated purity by LCMS analysis was 93.5%. Analysis condition B: Retention time=1.9 min; ESI-MS(+) m/z [M+2H]²⁺: 1223.4.

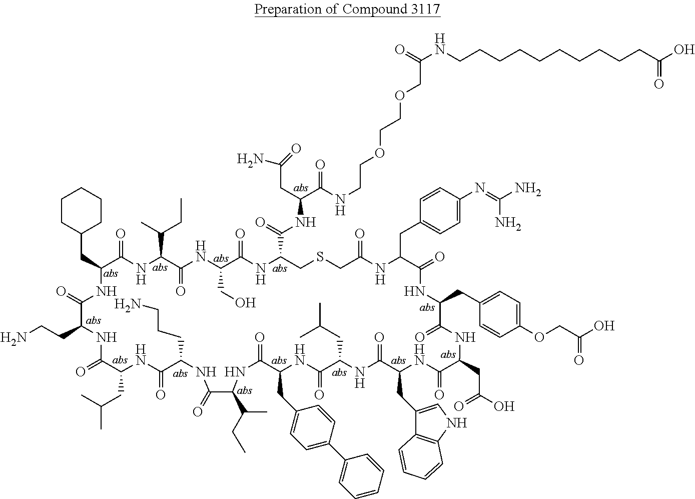 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C(Cc2ccc(N=C(N)N)cc2)NC(=O)CSC[C@@H](C(=O)N[C@@H](CC(N)=O)C(=O)NCCOCCOCC(=O)NCCCCCCCCCCC(=O)O)NC(=O)[C@H](CO)NC1=O]

Compound 3117 was prepared on a 50 μmol scale. The yield of the product was 13 mg, and its estimated purity by LCMS analysis was 97.7%. Analysis condition B: Retention time=1.75 min; ESI-MS(+) m/z [M+2H]²⁺: 1231.2.

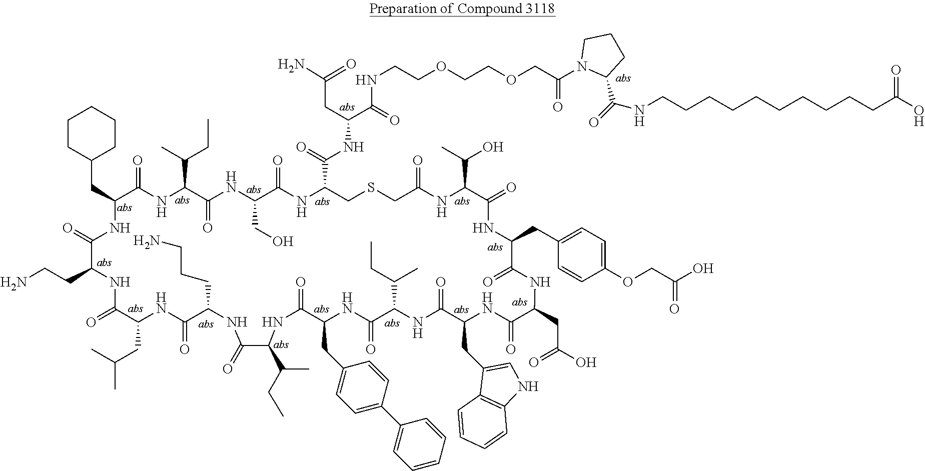 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)N2CCC[C@@H]2C(=O)NCCCCCCCCCCC(=O)O)NC(=O)[C@H](CO)NC1=O]

Compound 3118 was prepared on a 50 μmol scale. The yield of the product was 6.8 mg, and its estimated purity by LCMS analysis was 84.8%. Analysis condition B: Retention time=1.84 min; ESI-MS(+) m/z [M+2H]²⁺: 1228.1.

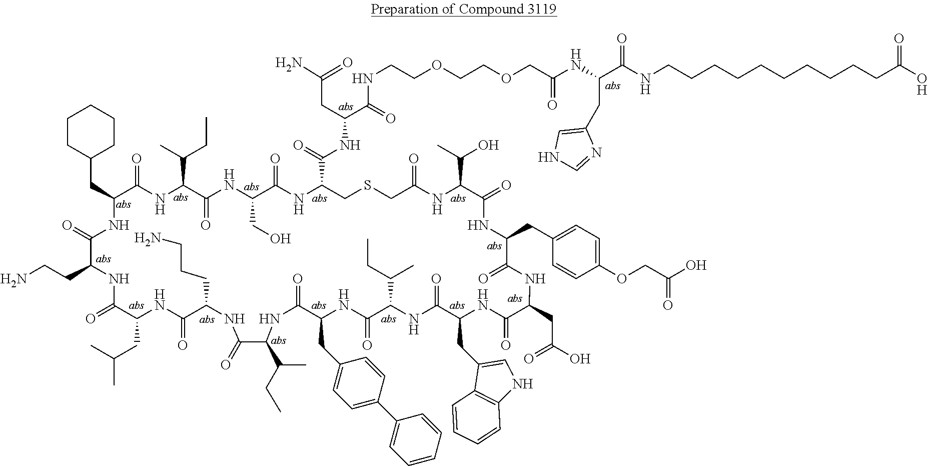 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)N[C@@H](Cc2c[nH]cn2)C(=O)NCCCCCCCCCCC(=O)O)NC(=O)[C@H](CO)NC1=O]

Compound 3119 was prepared on a 50 μmol scale. The yield of the product was 9.1 mg, and its estimated purity by LCMS analysis was 88.1%. Analysis condition A: Retention time=1.68 min; ESI-MS(+) m/z [M+3H]³⁺: 832.6.

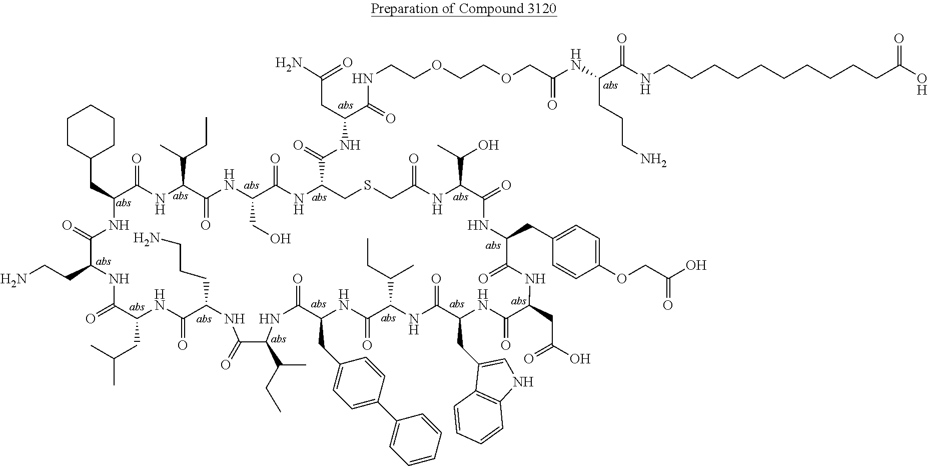 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCOCCOCC(=O)N[C@@H](CCCN)C(=O)NCCCCCCCCCCC(=O)O)NC(=O)[C@H](CO)NC1=O]

Compound 3120 was prepared on a 50 μmol scale. The yield of the product was 9 mg, and its estimated purity by LCMS analysis was 88.8%. Analysis condition B: Retention time=1.69 min; ESI-MS(+) m/z [M+2H]²⁺: 1236.1.

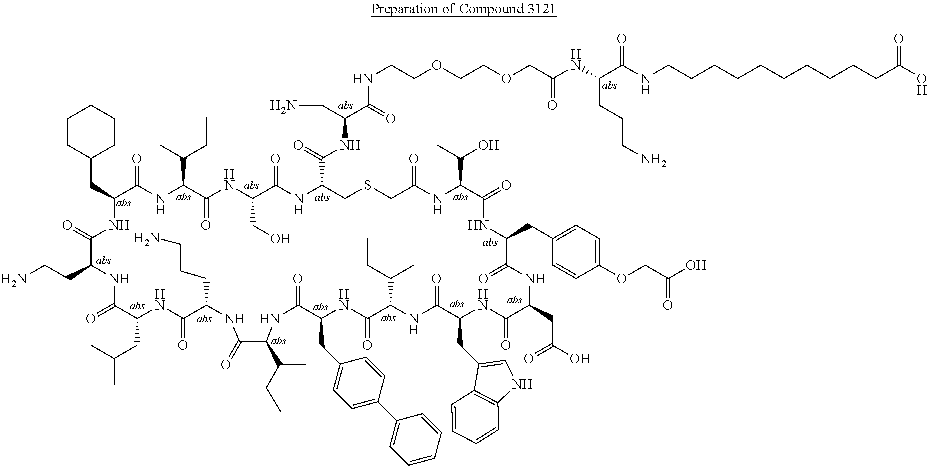 [SMILES: CCC(C)[C@@H]1NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](C(C)CC)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CN)C(=O)NCCOCCOCC(=O)N[C@@H](CCCN)C(=O)NCCCCCCCCCCC(=O)O)NC(=O)[C@H](CO)NC1=O]

Compound 3121 was prepared on a 50 μmol scale. The yield of the product was 8.9 mg, and its estimated purity by LCMS analysis was 98.7%. Analysis condition B: Retention time=1.64 min; ESI-MS(+) m/z [M+2H]²⁺: 1222.3.

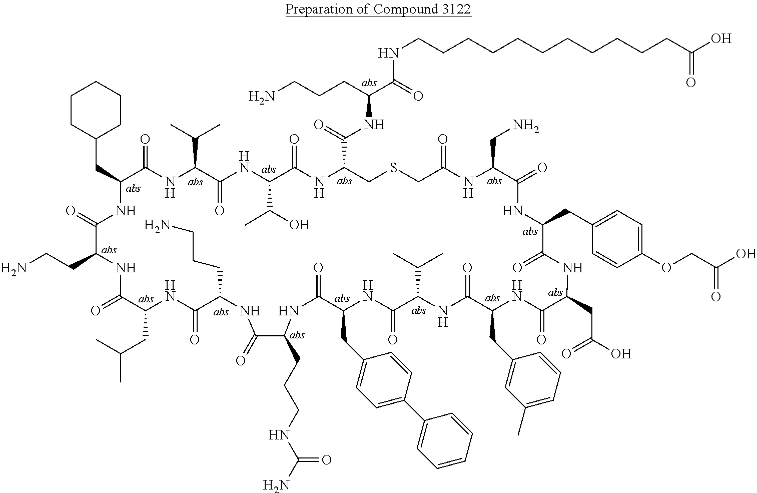 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3122 was prepared on a 50 μmol scale. The yield of the product was 8.4 mg, and its estimated purity by LCMS analysis was 96.2%. Analysis condition A: Retention time=1.65 min; ESI-MS(+) m/z [M+2H]²⁺: 1109.1.

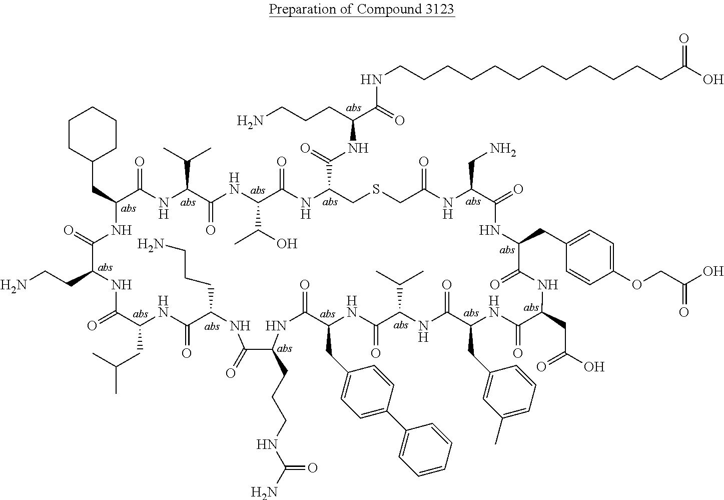 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)NCCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3123 was prepared on a 50 μmol scale. The yield of the product was 2.4 mg, and its estimated purity by LCMS analysis was 91.3%. Analysis condition B: Retention time=1.64 min; ESI-MS(+) m/z [M+2H]²⁺: 1116.

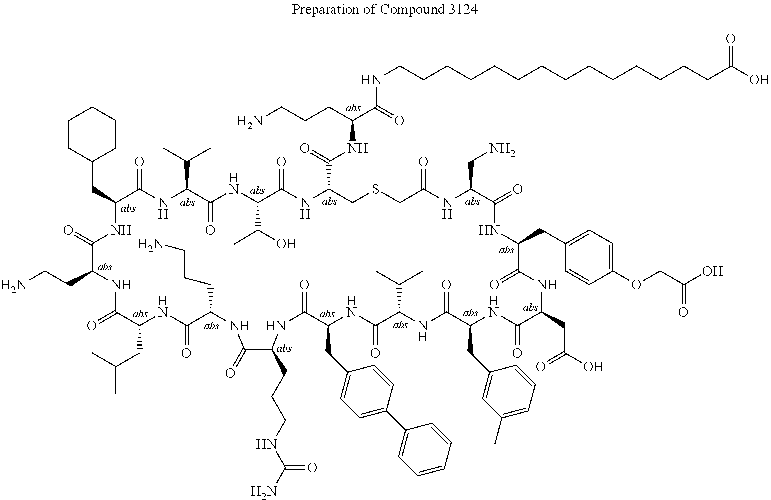 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)NCCCCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3124 was prepared on a 50 μmol scale. The yield of the product was 3.8 mg, and its estimated purity by LCMS analysis was 84.8%. Analysis condition B: Retention time=1.74 min; ESI-MS(+) m/z [M+2H]²⁺: 1130.

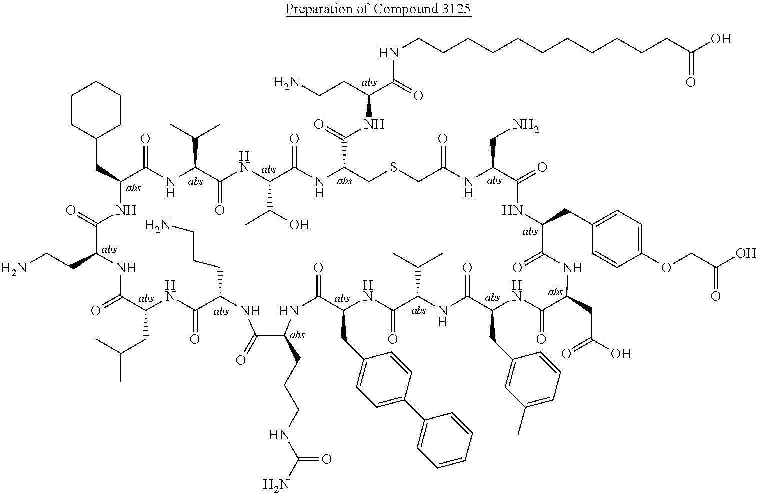 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3125 was prepared on a 50 μmol scale. The yield of the product was 13.4 mg, and its estimated purity by LCMS analysis was 86.7%. Analysis condition A: Retention time=1.66 min; ESI-MS(+) m/z [M+2H]²⁺: 1102.2.

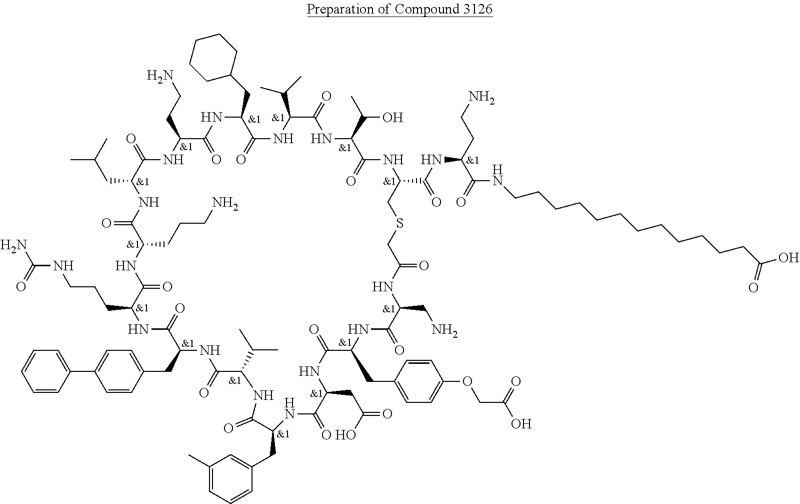 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3126 was prepared on a 50 μmol scale. The yield of the product was 6.4 mg, and its estimated purity by LCMS analysis was 85.2%. Analysis condition A: Retention time=1.71 min; ESI-MS(+) m/z [M+2H]²⁺: 1109.2.

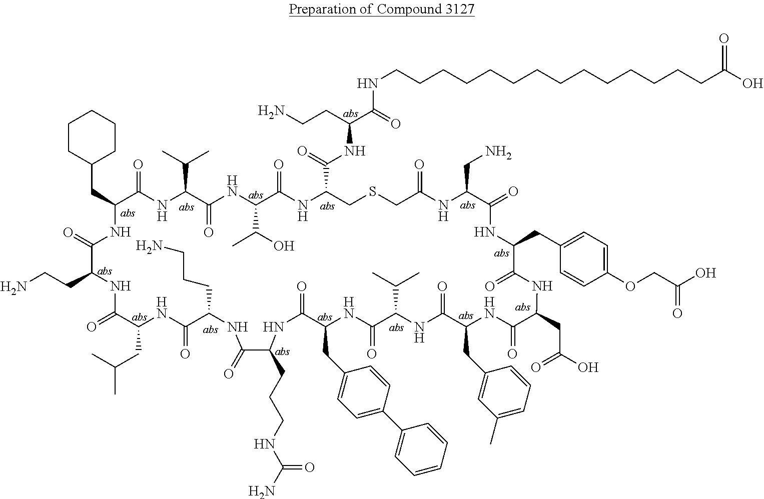 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3127 was prepared on a 50 μmol scale. The yield of the product was 3.5 mg, and its estimated purity by LCMS analysis was 97.3%. Analysis condition A: Retention time=1.86 min; ESI-MS(+) m/z [M+2H]²⁺: 1123.2.

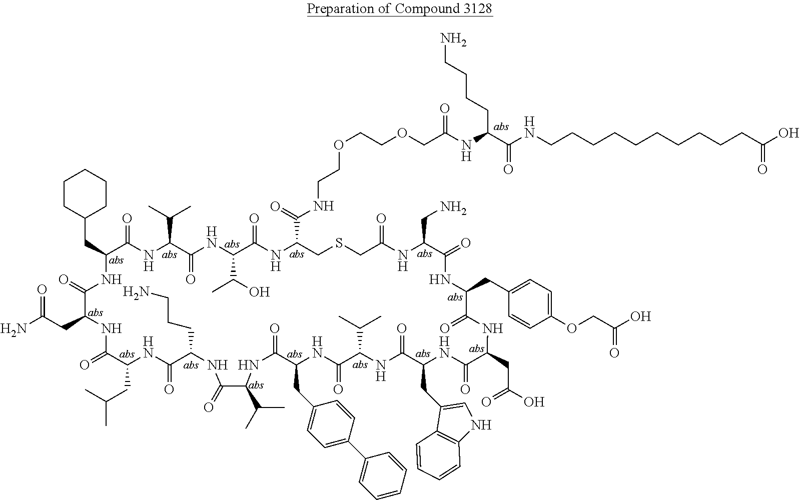 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CC(N)=O)NC1=O]

Compound 3128 was prepared on a 50 μmol scale. The yield of the product was 5.4 mg, and its estimated purity by LCMS analysis was 96.3%. Analysis condition B: Retention time=1.71 min; ESI-MS(+) m/z [M+2H]²⁺: 1172.2.

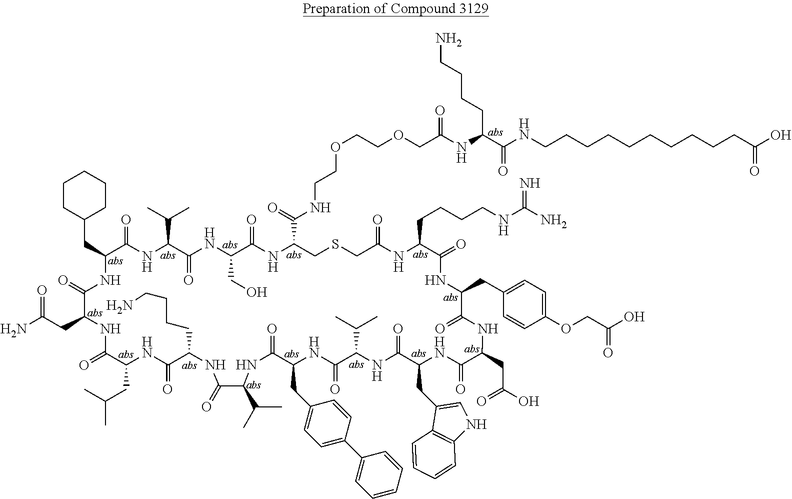 [SMILES: CC(C)C[C@H]1NC(=O)[C@H](CCCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC1=O]

Compound 3129 was prepared on a 50 μmol scale. The yield of the product was 10.1 mg, and its estimated purity by LCMS analysis was 100%. Analysis condition A: Retention time=1.57 min; ESI-MS(+) m/z [M+3H]³⁺: 810.2.

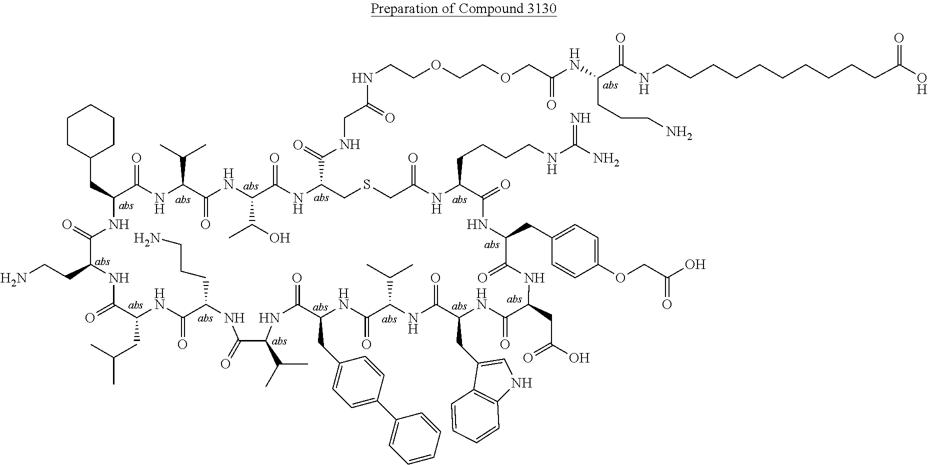 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3130 was prepared on a 50 μmol scale. The yield of the product was 13.8 mg, and its estimated purity by LCMS analysis was 100%. Analysis condition A: Retention time=1.57 min; ESI-MS(+) m/z [M+2H]²⁺: 1228.2.

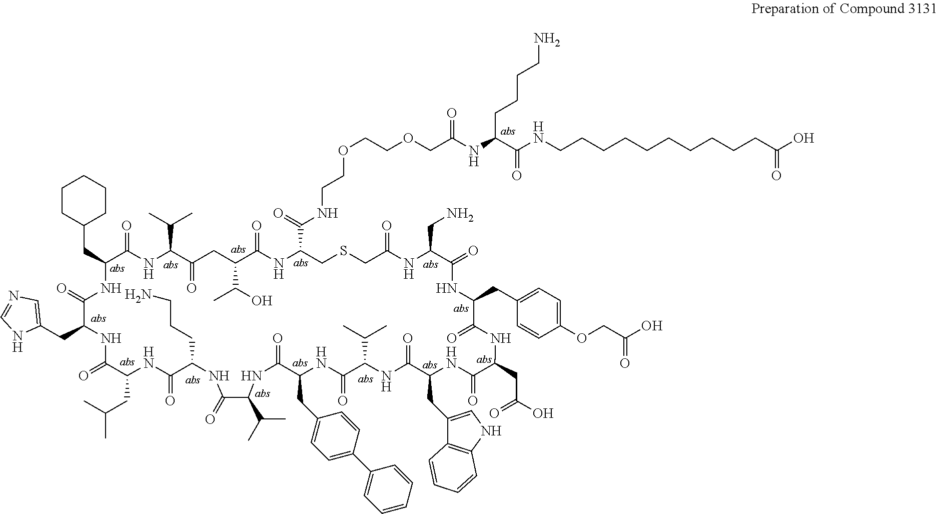 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](Cc2cnc[nH]2)NC1=O]

Compound 3131 was prepared on a 50 μmol scale. The yield of the product was 4 mg, and its estimated purity by LCMS analysis was 100%. Analysis condition A: Retention time=1.54 min; ESI-MS(+) m/z [M+2H]²⁺: 1176.7.

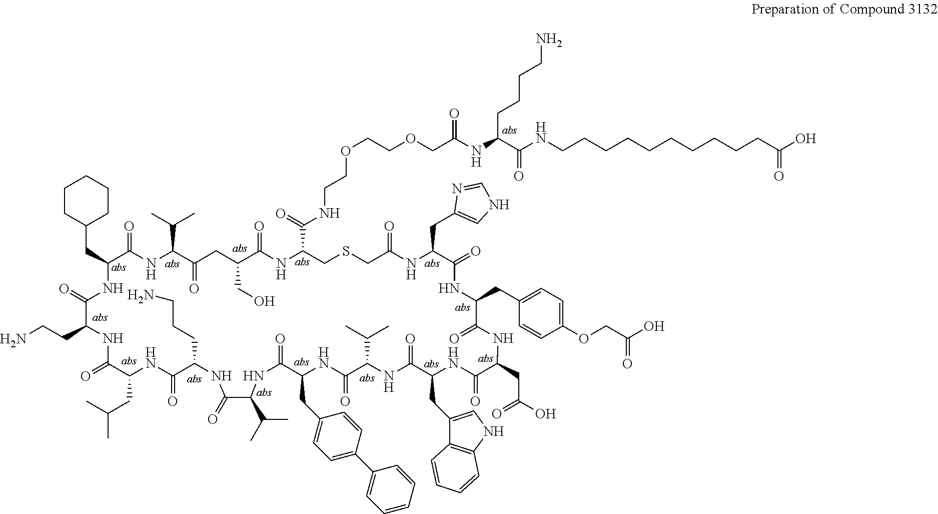 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2c[nH]cn2)NC(=O)CSC[C@@H](C(=O)NCCOCCOCC(=O)N[C@@H](CCCCN)C(=O)NCCCCCCCCCCC(=O)O)NC(=O)[C@H](CO)CC(=O)[C@H](C(C)C)NC(=O)[C@H](CC2CCCCC2)NC(=O)[C@H](CCN)NC1=O]

Compound 3132 was prepared on a 50 μmol scale. The yield of the product was 7.9 mg, and its estimated purity by LCMS analysis was 100%. Analysis condition A: Retention time=1.52 min; ESI-MS(+) m/z [M+3H]³⁺: 789.2.

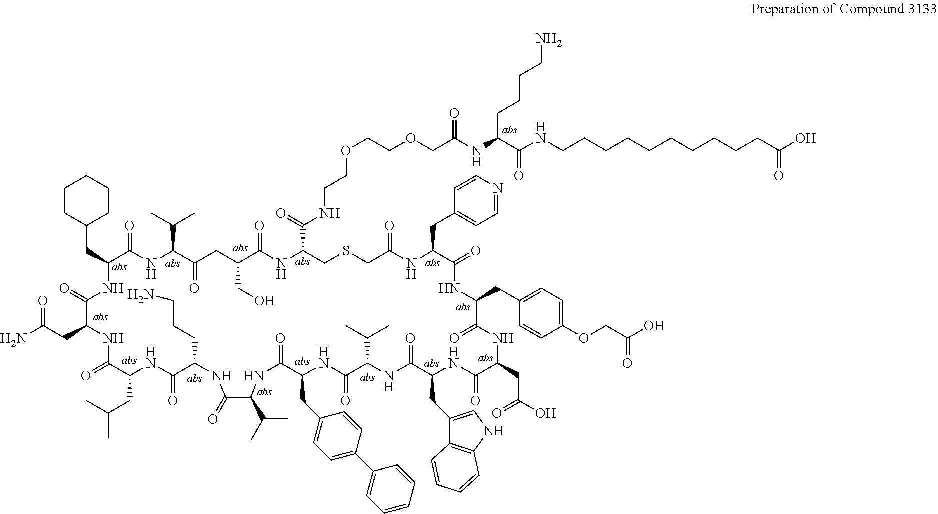 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCCOCCOCC(=O)N[C@@H](CCCCN)C(=O)NCCCCCCCCCCC(=O)O)NC(=O)[C@H](CO)CC(=O)[C@H](C(C)C)NC(=O)[C@H](CC2CCCCC2)NC(=O)[C@H](CC(N)=O)NC1=O]

Compound 3133 was prepared on a 50 μmol scale. The yield of the product was 13.1 mg, and its estimated purity by LCMS analysis was 95.9%. Analysis condition B: Retention time=1.66, 1.67 min; ESI-MS(+) m/z [M+3H]³⁺: 797.72, 797.72.

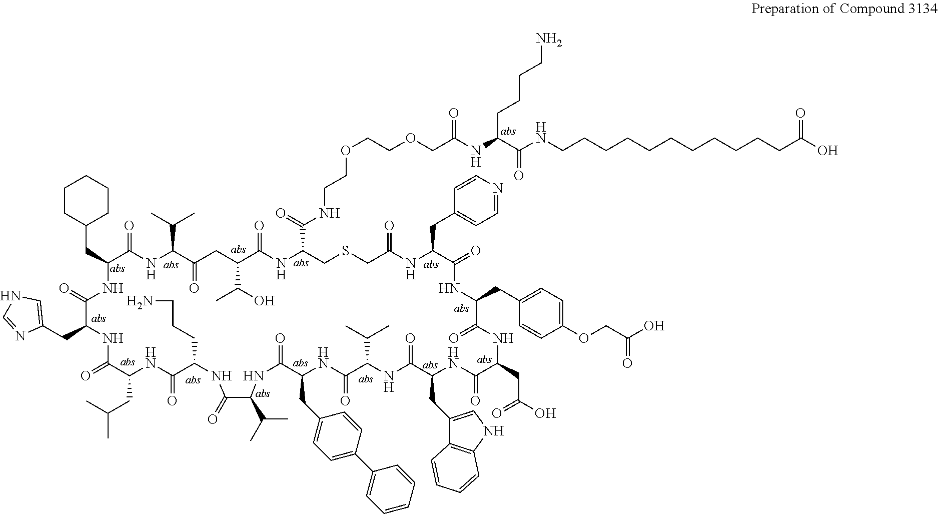 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCCOCCOCC(=O)N[C@@H](CCCCN)C(=O)NCCCCCCCCCCCC(=O)O)NC(=O)[C@H](C(C)O)CC(=O)[C@H](C(C)C)NC(=O)[C@H](CC2CCCCC2)NC(=O)[C@H](Cc2c[nH]cn2)NC1=O]

Compound 3134 was prepared on a 50 μmol scale. The yield of the product was 95.5 mg, and its estimated purity by LCMS analysis was 97%. Analysis condition A: Retention time=1.67 min; ESI-MS(+) m/z [M+2H]²⁺: 1190.2.

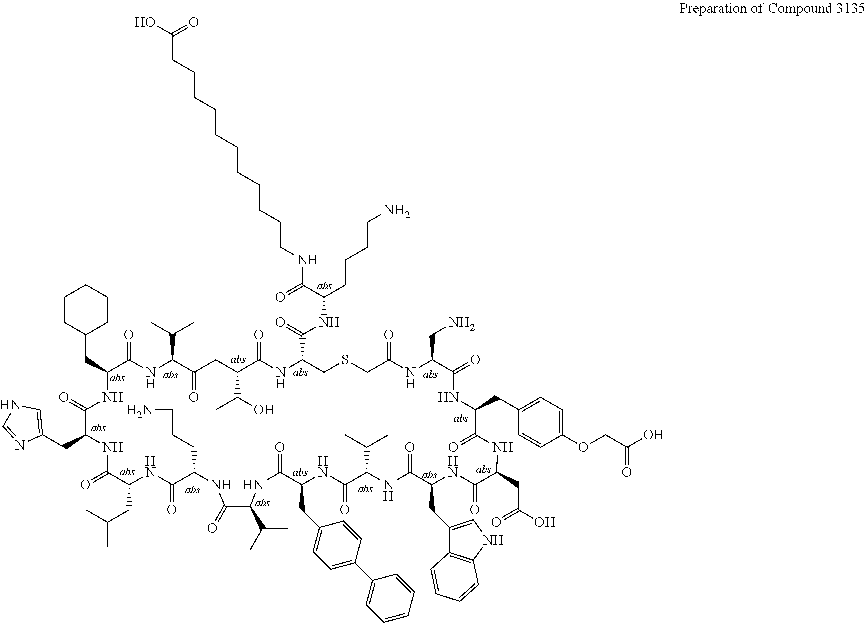 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCCC(=O)O)NC(=O)[C@H](C(C)O)CC(=O)[C@H](C(C)C)NC(=O)[C@H](CC2CCCCC2)NC(=O)[C@H](Cc2c[nH]cn2)NC1=O]

Compound 3135 was prepared on a 50 μmol scale. The yield of the product was 14.5 mg, and its estimated purity by LCMS analysis was 95.5%. Analysis condition B: Retention time=1.65 min; ESI-MS(+) m/z [M+2H]²⁺: 1117.4.

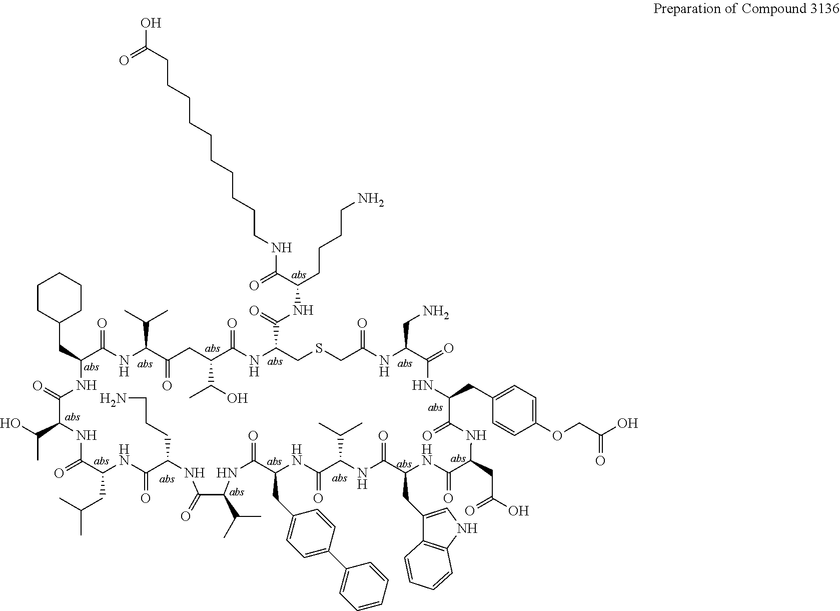 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](C(C)O)NC1=O]

Compound 3136 was prepared on a 50 μmol scale. The yield of the product was 8.2 mg, and its estimated purity by LCMS analysis was 95.5%. Analysis condition B: Retention time=1.74 min; ESI-MS(+) m/z [M+2H]²⁺: 1083.3.

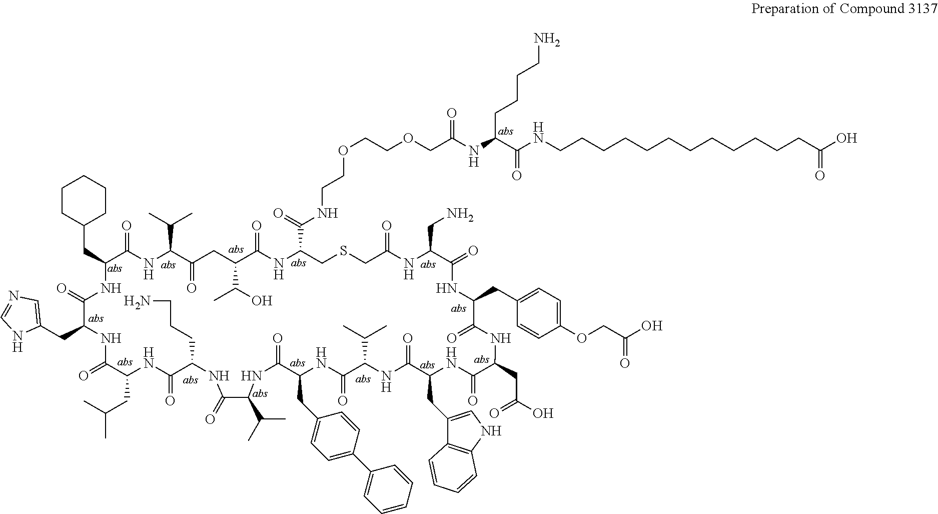 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCCN)C(=O)NCCCCCCCCCCCCC(=O)O)NC(=O)[C@H](C(C)O)CC(=O)[C@H](C(C)C)NC(=O)[C@H](CC2CCCCC2)NC(=O)[C@H](Cc2cnc[nH]2)NC1=O]

Compound 3137 was prepared on a 50 μmol scale. The yield of the product was 40 mg, and its estimated purity by LCMS analysis was 98.1%. Analysis condition B: Retention time=1.69 min; ESI-MS(+) m/z [M+2H]²⁺: 1196.9.

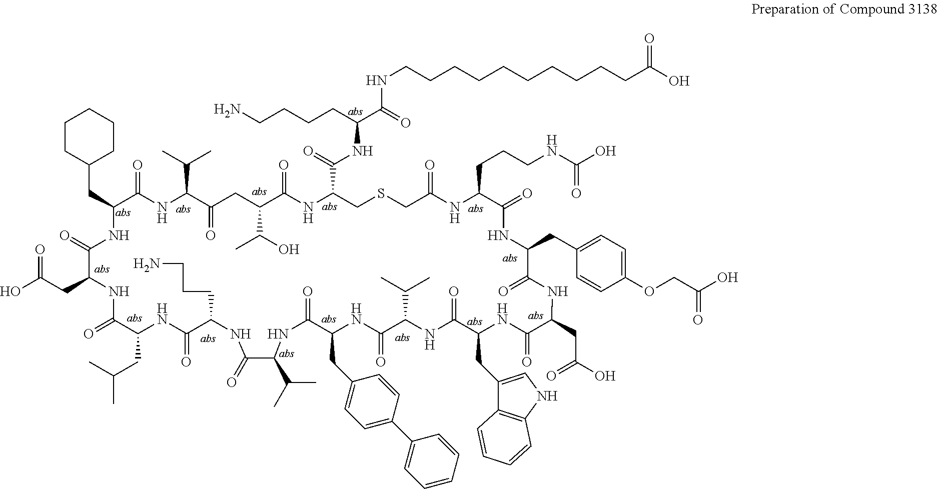 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=O)O)NC(=O)CSC[C@@H](C(=O)N[C@@H](CCCCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CC(=O)O)NC1=O]

Compound 3138 was prepared on a 50 μmol scale. The yield of the product was 21.2 mg, and its estimated purity by LCMS analysis was 97.4%. Analysis condition A: Retention time=1.69 min; ESI-MS(+) m/z [M+2H]²⁺: 1135.

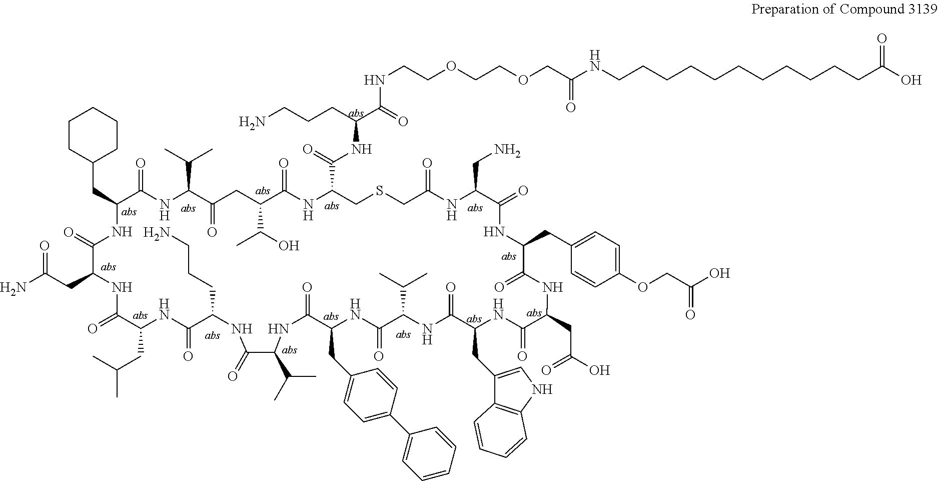 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCOCCOCC(=O)NCCCCCCCCCCCC(=O)O)NC(=O)[C@H](C(C)O)CC(=O)[C@H](C(C)C)NC(=O)[C@H](CC2CCCCC2)NC(=O)[C@H](CC(N)=O)NC1=O]

Compound 3139 was prepared on a 50 μmol scale. The yield of the product was 27.1 mg, and its estimated purity by LCMS analysis was 100%. Analysis condition A: Retention time=1.72 min; ESI-MS(+) m/z [M+2H]²⁺: 1172.

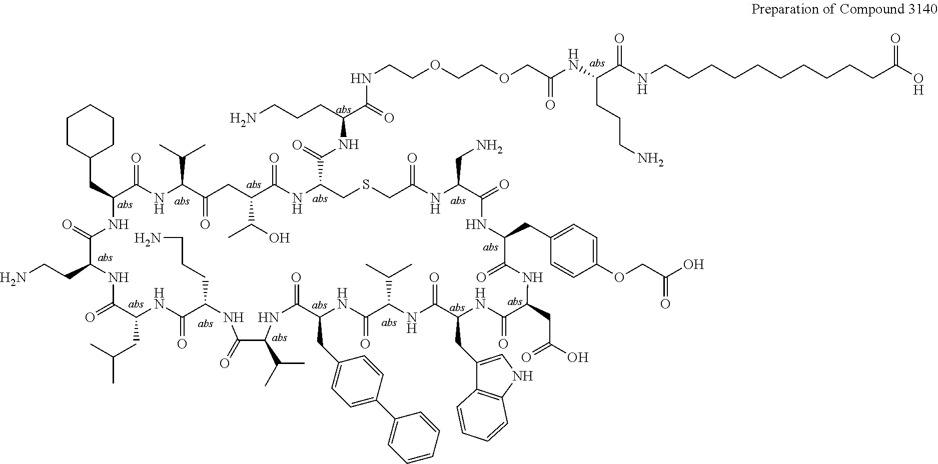 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCOCCOCC(=O)N[C@@H](CCCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CCN)NC1=O]

Compound 3140 was prepared on a 50 μmol scale. The yield of the product was 20.3 mg, and its estimated purity by LCMS analysis was 95.5%. Analysis condition B: Retention time=1.5 min; ESI-MS(+) m/z [M+2H]²⁺: 1208.1.

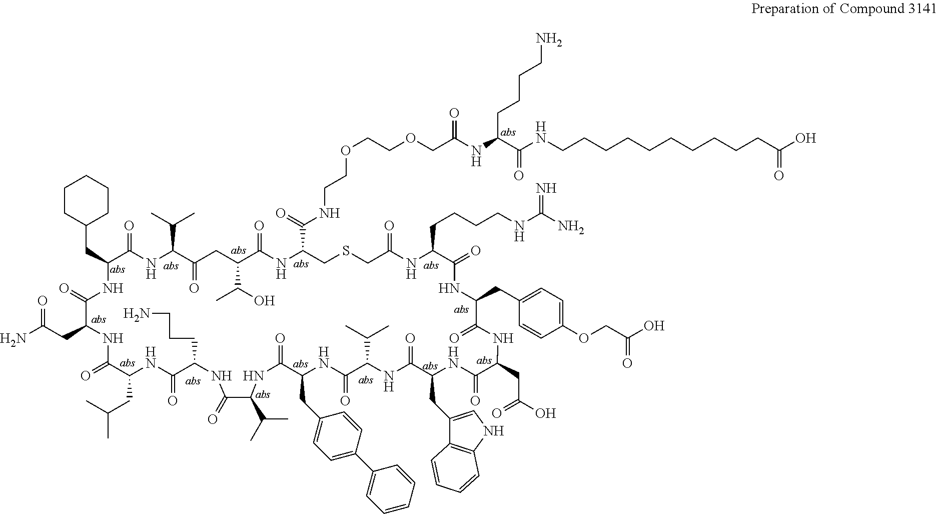 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CC(N)=O)NC1=O]

Compound 3141 was prepared on a 50 μmol scale. The yield of the product was 14.8 mg, and its estimated purity by LCMS analysis was 99.2%. Analysis condition A: Retention time=1.74 min; ESI-MS(+) m/z [M+2H]²⁺: 1214.2.

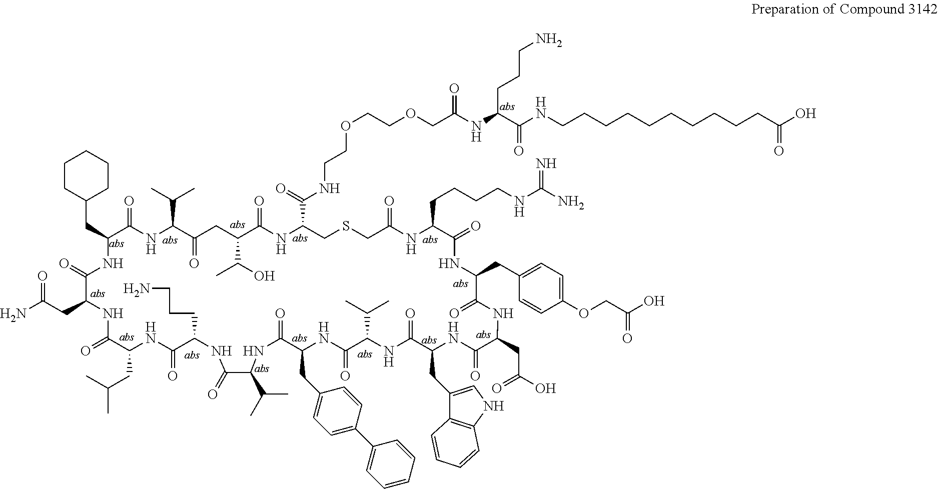 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CC(N)=O)NC1=O]

Compound 3142 was prepared on a 50 μmol scale. The yield of the product was 16.4 mg, and its estimated purity by LCMS analysis was 96%. Analysis condition A: Retention time=1.74 min; ESI-MS(+) m/z [M+2H]²⁺: 1207.2.

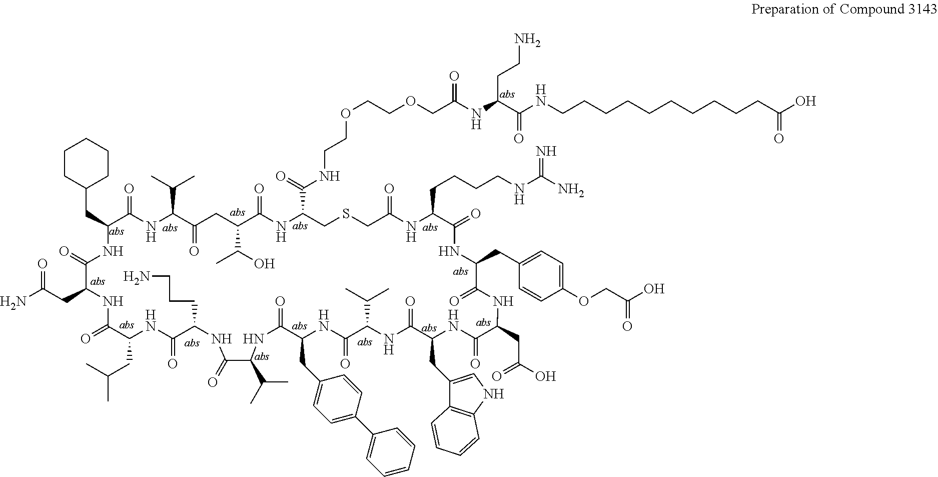 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CC(N)=O)NC1=O]

Compound 3143 was prepared on a 50 μmol scale. The yield of the product was 33.5 mg, and its estimated purity by LCMS analysis was 98%. Analysis condition B: Retention time=1.75 min; ESI-MS(+) m/z [M+2H]²⁺: 1200.1.

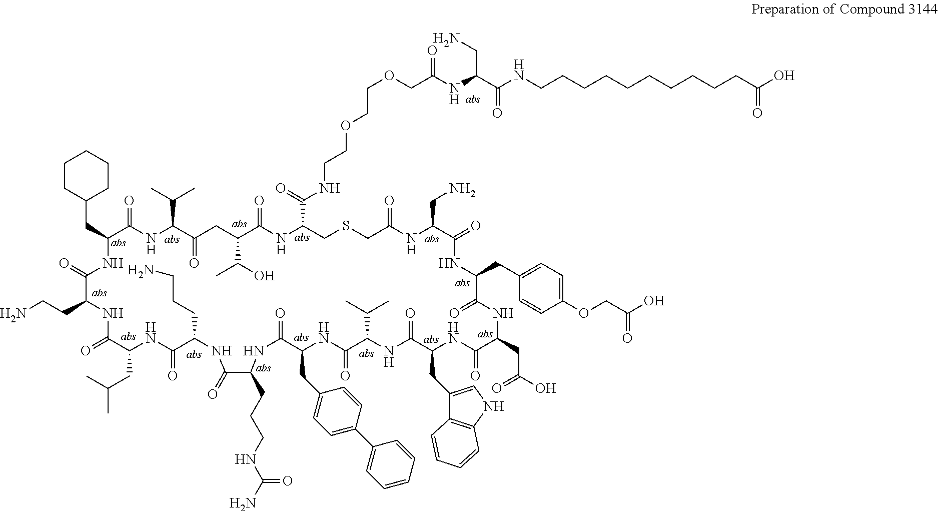 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CCN)NC1=O]

Compound 3144 was prepared on a 50 μmol scale. The yield of the product was 29.6 mg, and its estimated purity by LCMS analysis was 100%. Analysis condition A: Retention time=1.6 min; ESI-MS(+) m/z [M+2H]²⁺: 1173.1.

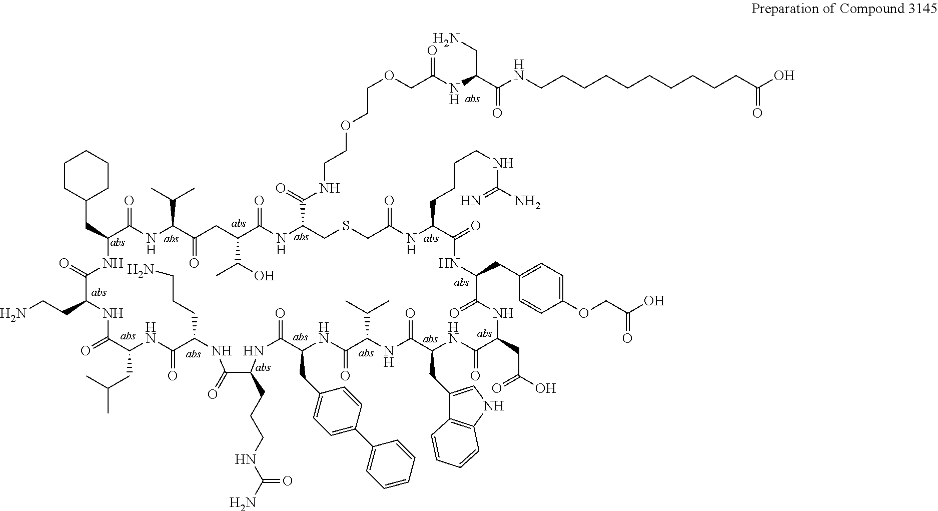 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CN)C(=O)NCCCCCCCCCCC(=O)O)NC(=O)[C@H](C(C)O)CC(=O)[C@H](C(C)C)NC(=O)[C@H](CC2CCCCC2)NC(=O)[C@H](CCN)NC1=O]

Compound 3145 was prepared on a 50 μmol scale. The yield of the product was 25.1 mg, and its estimated purity by LCMS analysis was 96.9%. Analysis condition B: Retention time=1.56 min; ESI-MS(+) m/z [M+2H]²⁺: 1215.1.

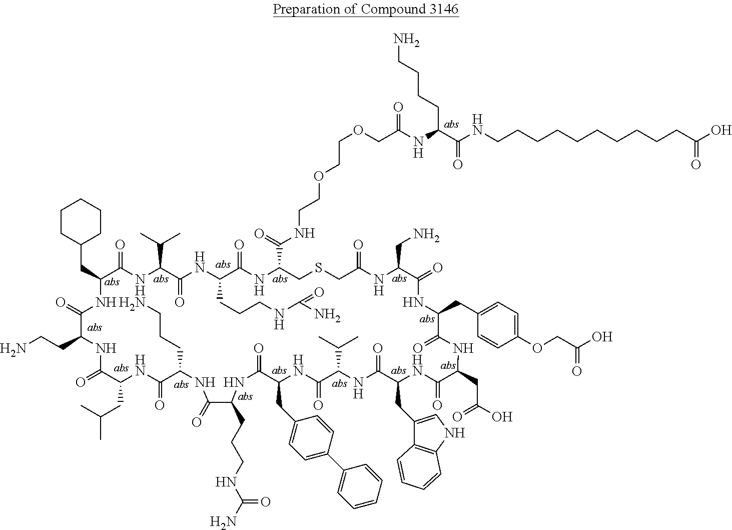 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC1=O]

Compound 3146 was prepared on a 50 μmol scale. The yield of the product was 14.2 mg, and its estimated purity by LCMS analysis was 100%. Analysis condition A: Retention time=1.53 min; ESI-MS(+) m/z [M+3H]³⁺: 815.

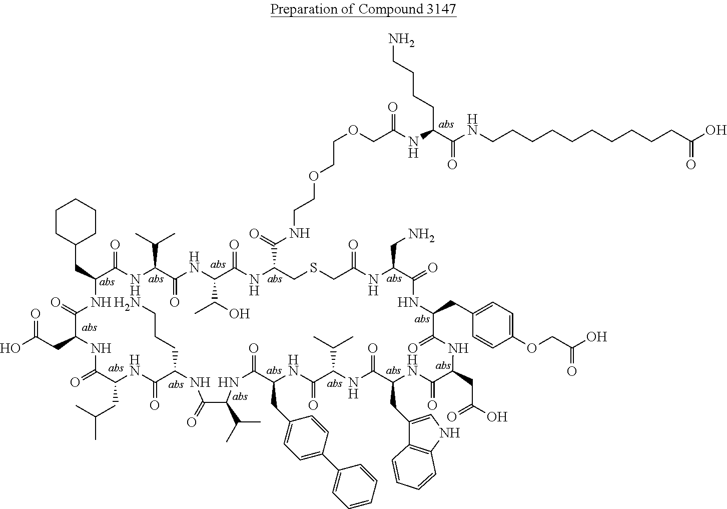 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CC(=O)O)NC1=O]

Compound 3147 was prepared on a 50 μmol scale. The yield of the product was 8.9 mg, and its estimated purity by LCMS analysis was 95.3%. Analysis condition B: Retention time=1.61 min; ESI-MS(+) m/z [M+3H]³⁺: 782.2.

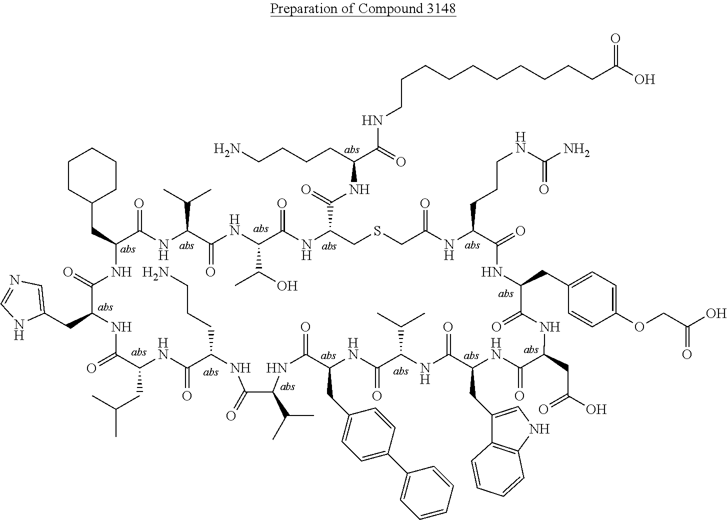 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(N)=O)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](Cc2cnc[nH]2)NC1=O]

Compound 3148 was prepared on a 50 μmol scale. The yield of the product was 43.9 mg, and its estimated purity by LCMS analysis was 96.2%. Analysis condition A: Retention time=1.59 min; ESI-MS(+) m/z [M+3H]³⁺: 765.1.

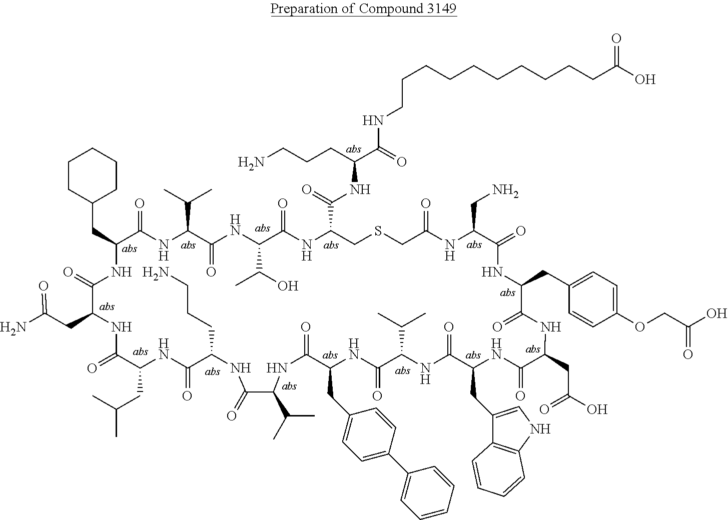 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CC(N)=O)NC1=O]

Compound 3149 was prepared on a 50 μmol scale. The yield of the product was 37.1 mg, and its estimated purity by LCMS analysis was 95.2%. Analysis condition B: Retention time=1.73 min; ESI-MS(+) m/z [M+2H]²⁺: 1092.2.

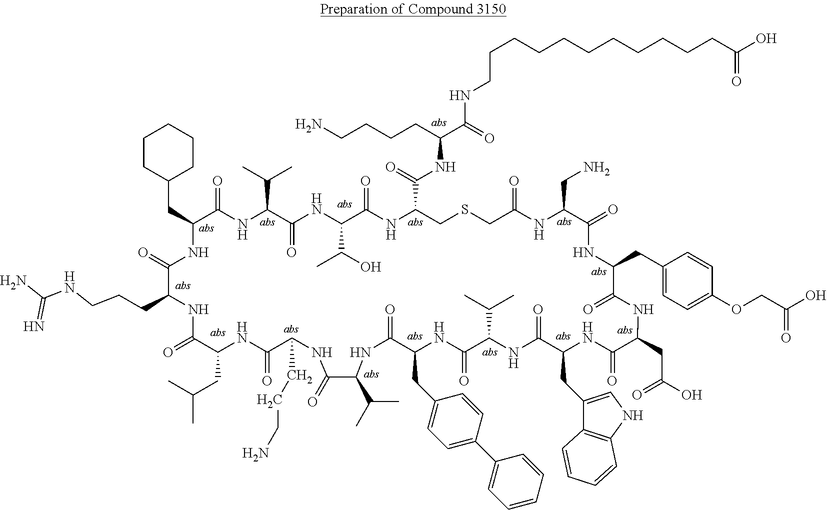 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCCN)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCCNC(=N)N)NC1=O]

Compound 3150 was prepared on a 50 μmol scale. The yield of the product was 37.2 mg, and its estimated purity by LCMS analysis was 97.7%. Analysis condition B: Retention time=1.59 min; ESI-MS(+) m/z [M+3H]³⁺: 752.2.

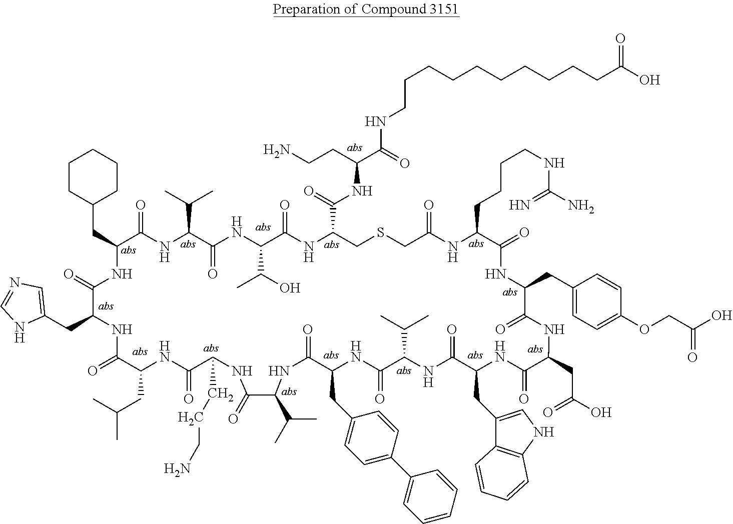 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](Cc2cnc[nH]2)NC1=O]

Compound 3151 was prepared on a 50 μmol scale. The yield of the product was 39 mg, and its estimated purity by LCMS analysis was 96.4%. Analysis condition B: Retention time=1.45 min; ESI-MS(+) m/z [M+3H]³⁺: 760.1.

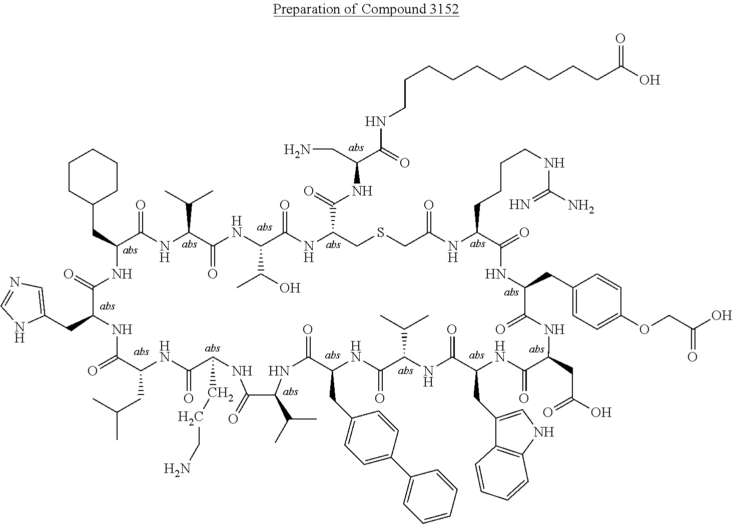 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](Cc2cnc[nH]2)NC1=O]

Compound 3152 was prepared on a 50 μmol scale. The yield of the product was 30.9 mg, and its estimated purity by LCMS analysis was 97.7%. Analysis condition A: Retention time=1.6, 1.66 min; ESI-MS(+) m/z [M+2H]²⁺: 1132.25, 1132.02.

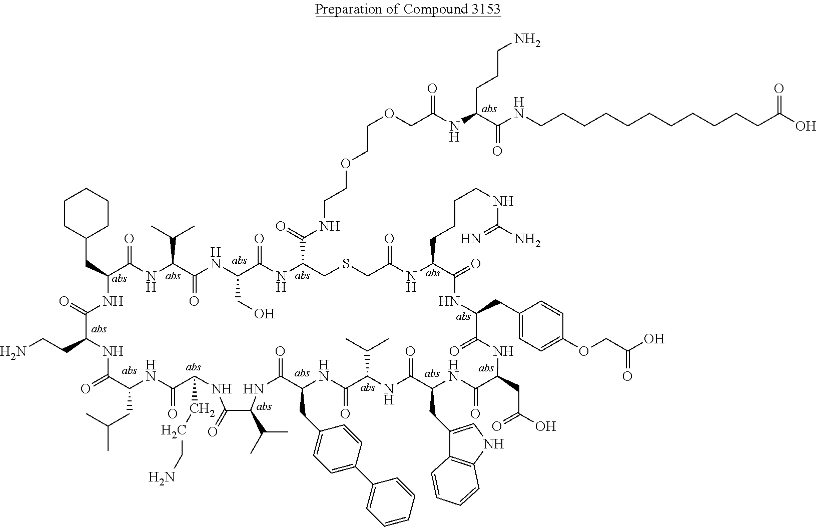 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3153 was prepared on a 50 μmol scale. The yield of the product was 22.6 mg, and its estimated purity by LCMS analysis was 95.8%. Analysis condition B: Retention time=1.62 min; ESI-MS(+) m/z [M+2H]²⁺: 1200.1.

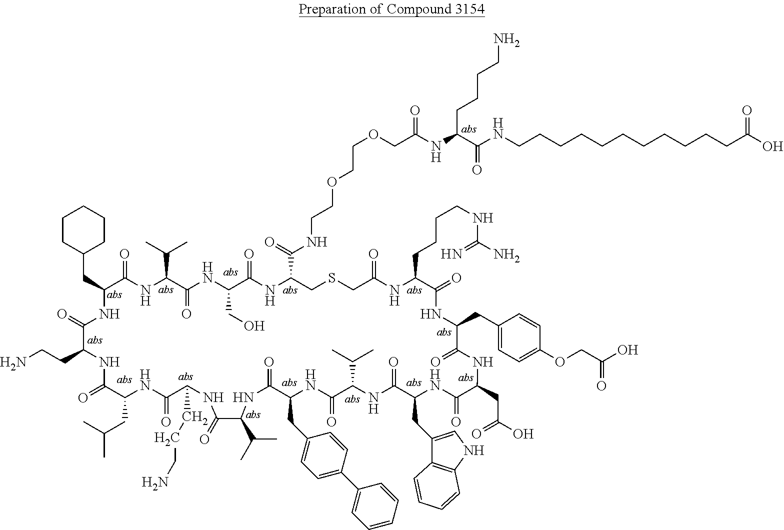 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3154 was prepared on a 50 μmol scale. The yield of the product was 20.2 mg, and its estimated purity by LCMS analysis was 90.7%. Analysis condition B: Retention time=1.62 min; ESI-MS(+) m/z [M+3H]³⁺: 804.9.

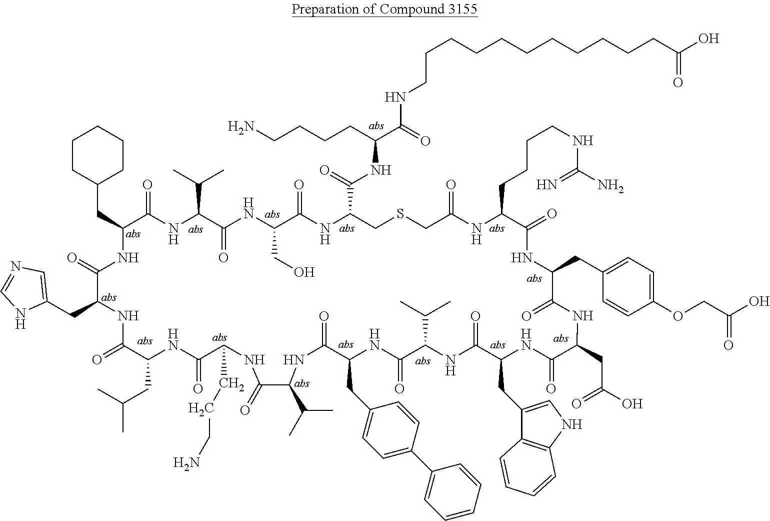 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](Cc2cnc[nH]2)NC1=O]

Compound 3155 was prepared on a 50 μmol scale. The yield of the product was 13.8 mg, and its estimated purity by LCMS analysis was 96.1%. Analysis condition B: Retention time-1.38 min; ESI-MS(+) m/z [M+3H]³⁺: 769.

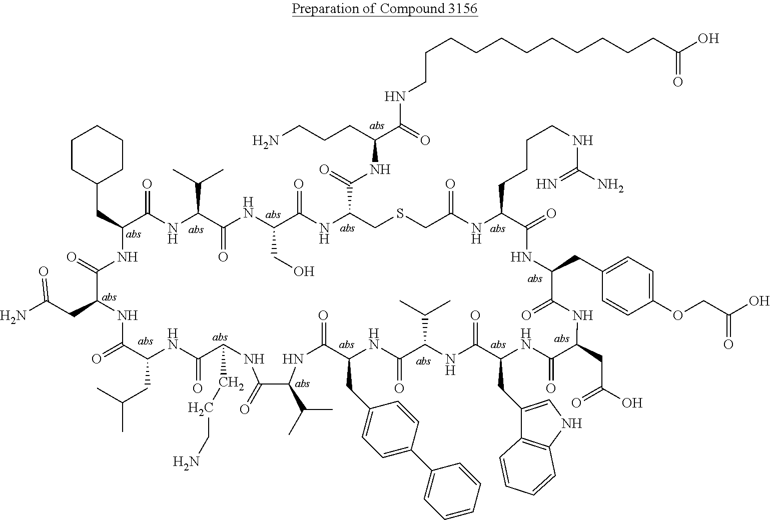 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC1=O]

Compound 3156 was prepared on a 50 μmol scale. The yield of the product was 12.6 mg, and its estimated purity by LCMS analysis was 95.5%. Analysis condition B: Retention time=1.71 min; ESI-MS(+) m/z [M+3H]³⁺: 756.9.

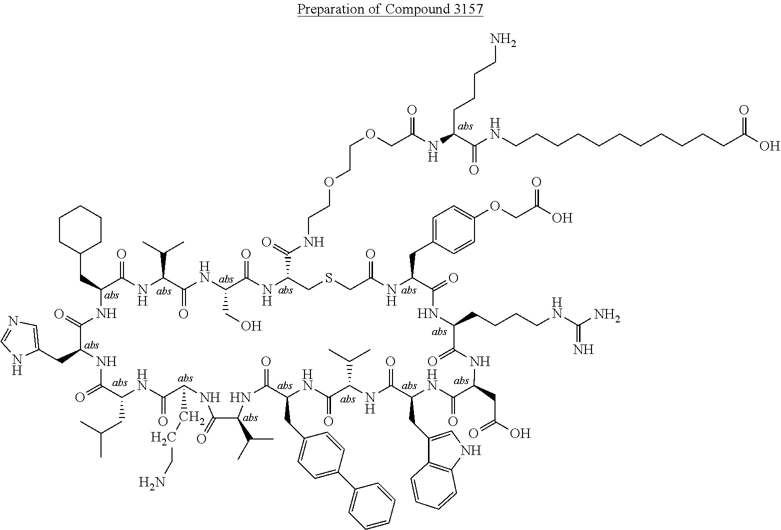 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCCCNC(=N)N)NC(=O)[C@H](Cc2ccc(OCC(=O)O)cc2)NC(=O)CSC[C@@H](C(=O)NCCOCCOCC(=O)N[C@@H](CCCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](Cc2cnc[nH]2)NC1=O]

Compound 3157 was prepared on a 50 μmol scale. The yield of the product was 36.4 mg, and its estimated purity by LCMS analysis was 98.1%. Analysis condition A: Retention time=1.68 min; ESI-MS(+) m/z [M+3H]³⁺: 815.3.

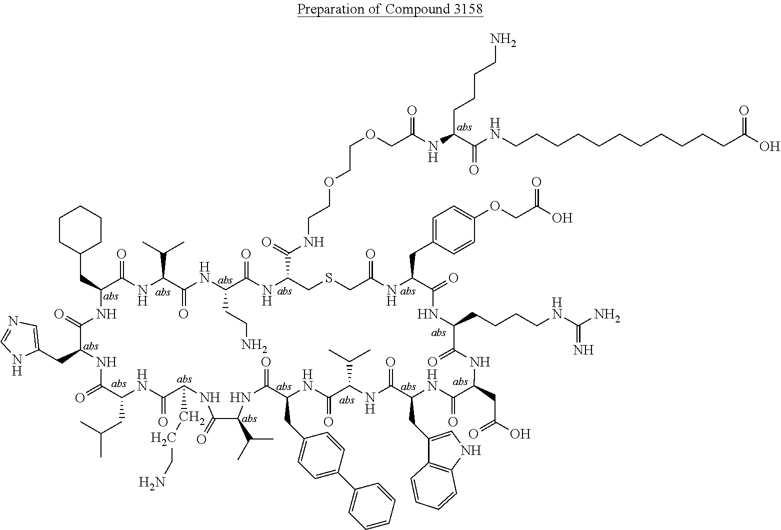 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCCCNC(=N)N)NC(=O)[C@H](Cc2ccc(OCC(=O)O)cc2)NC(=O)CSC[C@@H](C(=O)NCCOCCOCC(=O)N[C@@H](CCCCN)C(=O)NCCCCCCCCCCCC(=O)O)NC(=O)[C@H](CCN)NC(=O)[C@H](C(C)C)NC(=O)[C@H](CC2CCCCC2)NC(=O)[C@H](Cc2cnc[nH]2)NC1=O]

Compound 3158 was prepared on a 50 μmol scale. The yield of the product was 60.2 mg, and its estimated purity by LCMS analysis was 97.6%. Analysis condition B: Retention time=1.5 min; ESI-MS(+) m/z [M+2H]²⁺: 1232.

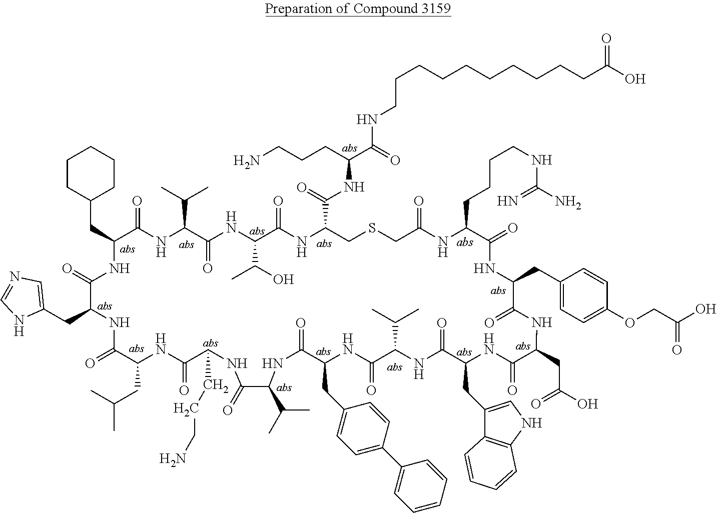 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](Cc2cnc[nH]2)NC1=O]

Compound 3159 was prepared on a 50 μmol scale. The yield of the product was 46.3 mg, and its estimated purity by LCMS analysis was 97.7%. Analysis condition B: Retention time=1.52, 1.56 min; ESI-MS(+) m/z [M+2H]²⁺: 1146.12, 1146.12.

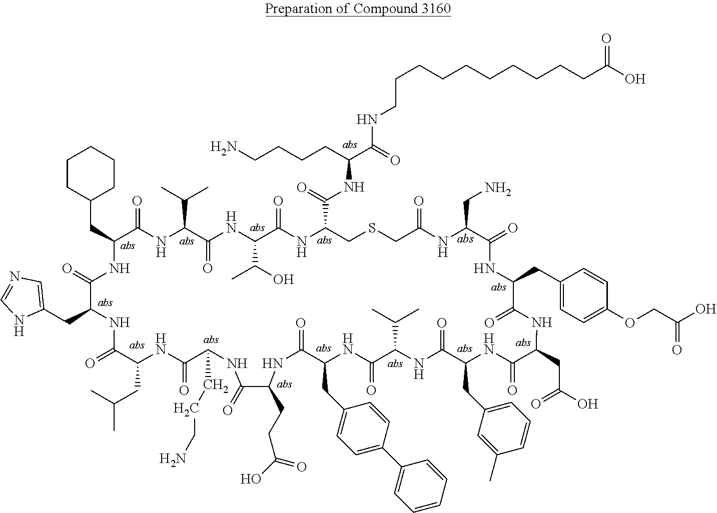 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 3160 was prepared on a 50 μmol scale. The yield of the product was 27.6 mg, and its estimated purity by LCMS analysis was 100%. Analysis condition A: Retention time=1.59 min; ESI-MS(+) m/z [M+3H]³⁺: 742.4.

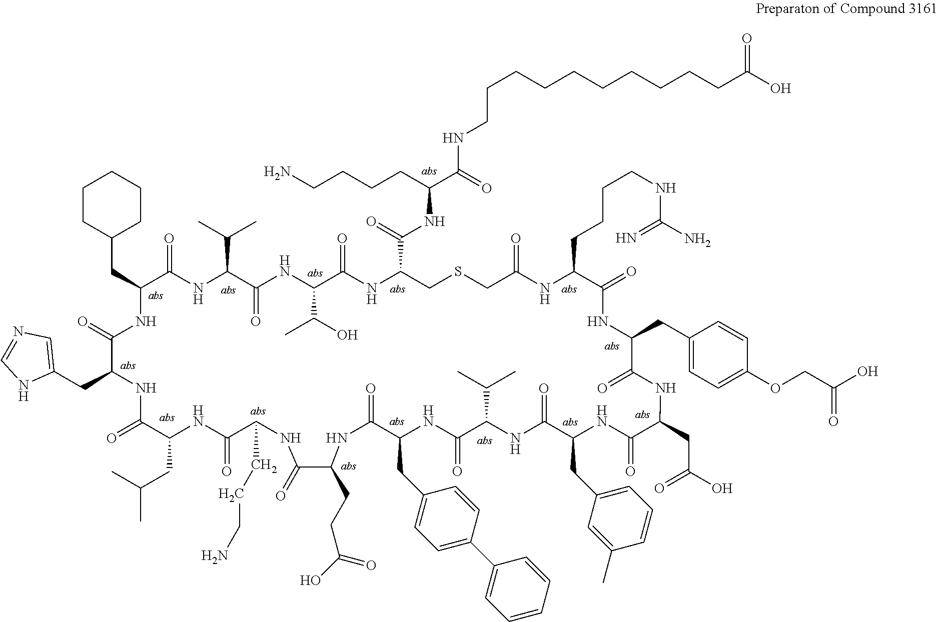 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 3161 was prepared on a 50 μmol scale. The yield of the product was 14.5 mg, and its estimated purity by LCMS analysis was 100%. Analysis condition A: Retention time=1.54 min; ESI-MS(+) m/z [M+3H]³⁺: 770.6.

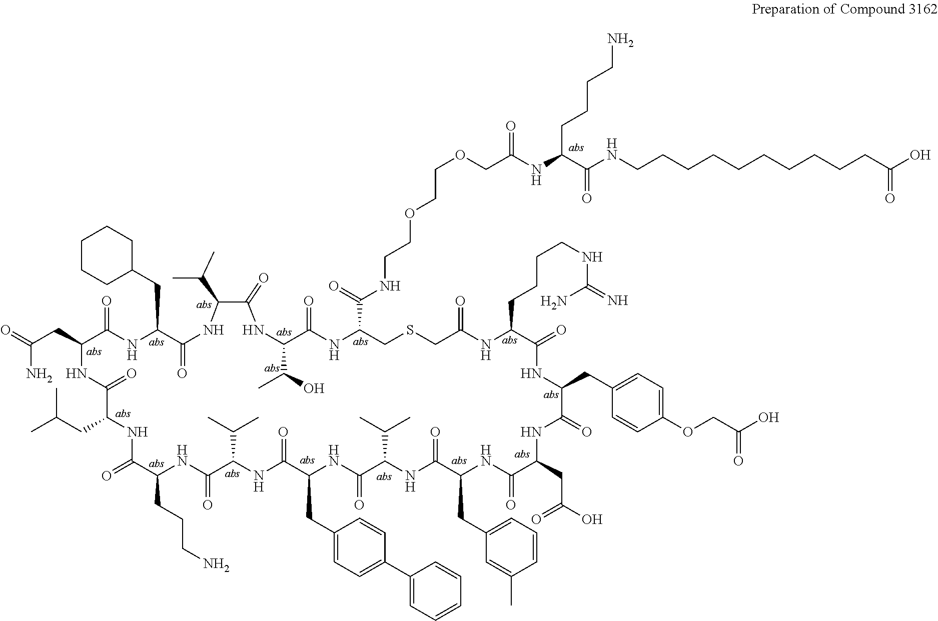 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCCOCCOCC(=O)N[C@@H](CCCCN)C(=O)NCCCCCCCCCCC(=O)O)NC(=O)[C@H]([C@H](C)O)NC(=O)[C@H](C(C)C)NC(=O)[C@H](CC3CCCCC3)NC(=O)[C@H](CC(N)=O)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3162 was prepared on a 50 μmol scale. The yield of the product was 37.8 mg, and its estimated purity by LCMS analysis was 100%. Analysis condition A: Retention time=1.75 min; ESI-MS(+) m/z [M+3H]³⁺: 801.3.

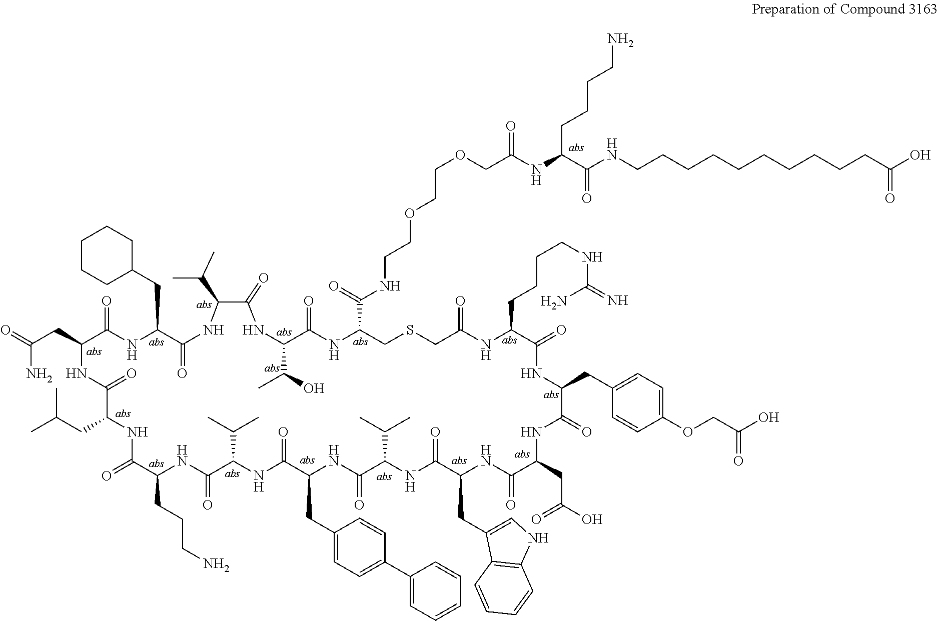 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCCN)C(=O)NCCCCCCCCCCC(=O)O)NC(=O)[C@H]([C@H](C)O)NC(=O)[C@H](C(C)C)NC(=O)[C@H](CC2CCCCC2)NC(=O)[C@H](CC(N)=O)NC1=O]

Compound 3163 was prepared on a 50 μmol scale. The yield of the product was 56.1 mg, and its estimated purity by LCMS analysis was 95.7%. Analysis condition B: Retention time=1.7 min; ESI-MS(+) m/z [M+3H]³⁺: 810.

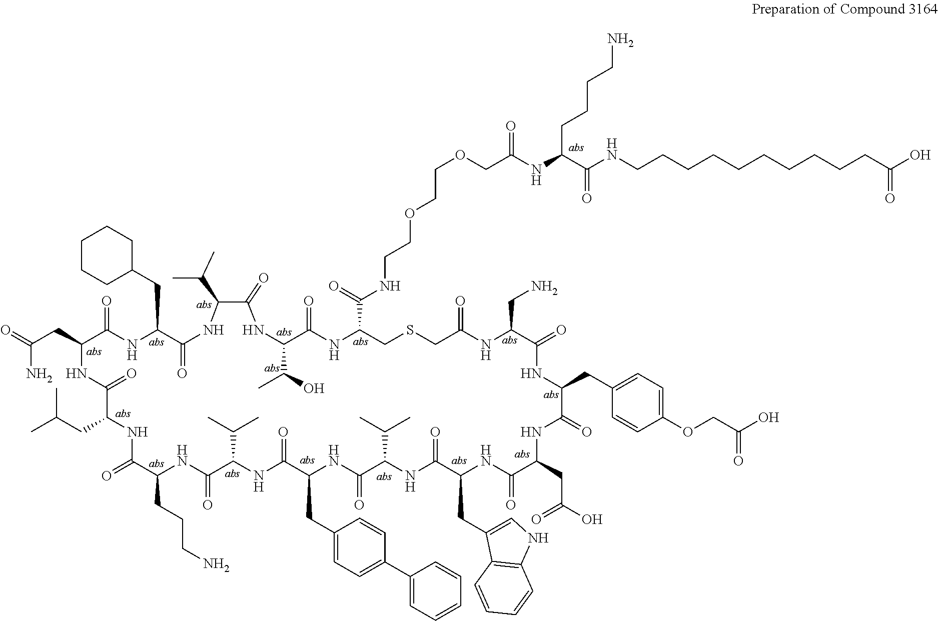 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCCN)C(=O)NCCCCCCCCCCC(=O)O)NC(=O)[C@H]([C@H](C)O)NC(=O)[C@H](C(C)C)NC(=O)[C@H](CC2CCCCC2)NC(=O)[C@H](CC(N)=O)NC1=O]

Compound 3164 was prepared on a 50 μmol scale. The yield of the product was 39.9 mg, and its estimated purity by LCMS analysis was 95.9%. Analysis condition A: Retention time=1.64 min; ESI-MS(+) m/z [M+2H]²⁺: 1172.1.

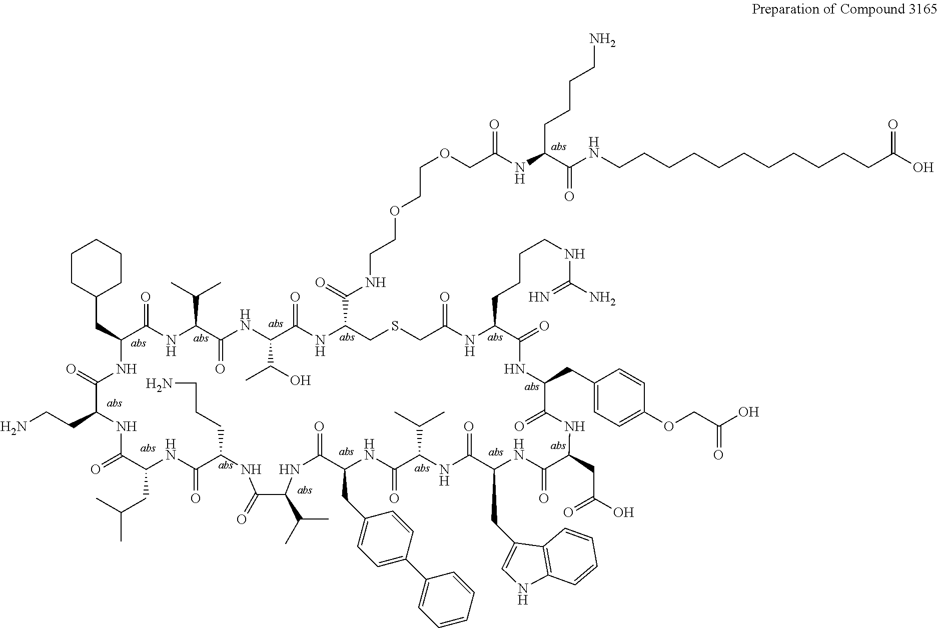 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCCN)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3165 was prepared on a 50 μmol scale. The yield of the product was 29.5 mg, and its estimated purity by LCMS analysis was 95.6%. Analysis condition B: Retention time=1.59 min; ESI-MS(+) m/z [M+2H]²⁺: 1214.1.

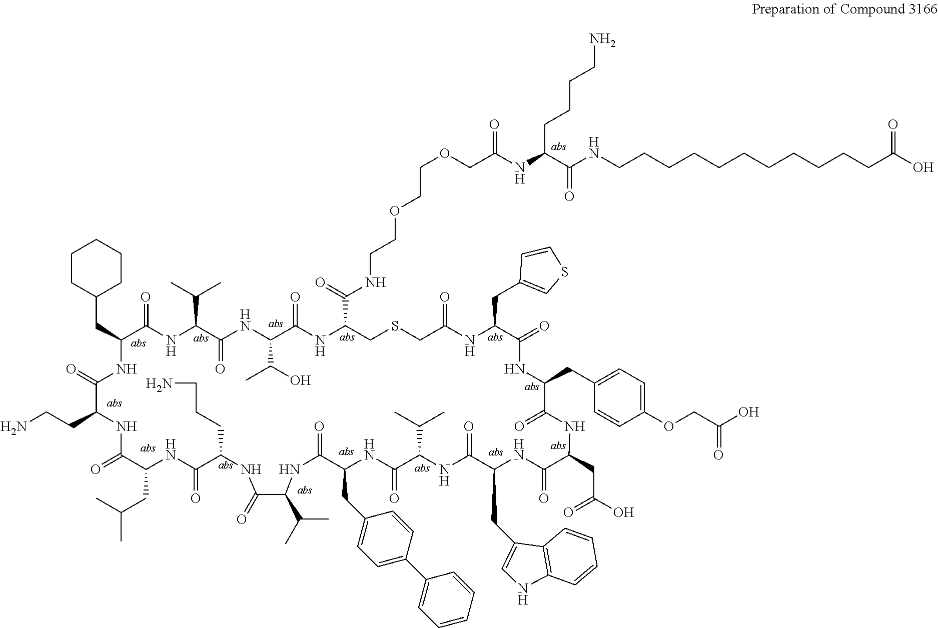 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)N[C@@H](CCCCN)C(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3166 was prepared on a 50 μmol scale. The yield of the product was 56.3 mg, and its estimated purity by LCMS analysis was 95.8%. Analysis condition A: Retention time=1.81 min; ESI-MS(+) m/z [M+3H]³⁺: 799.5.

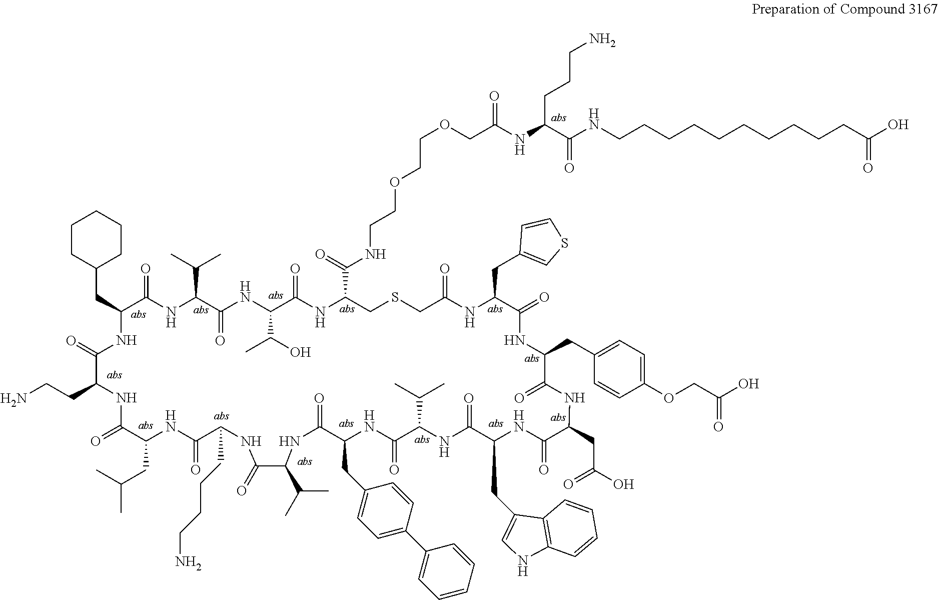 [SMILES: CC(C)C[C@H]1NC(=O)[C@H](CCCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3167 was prepared on a 50 μmol scale. The yield of the product was 33.4 mg, and its estimated purity by LCMS analysis was 96%. Analysis condition B: Retention time=1.7 min; ESI-MS(+) m/z [M+3H]³⁺: 799.1.

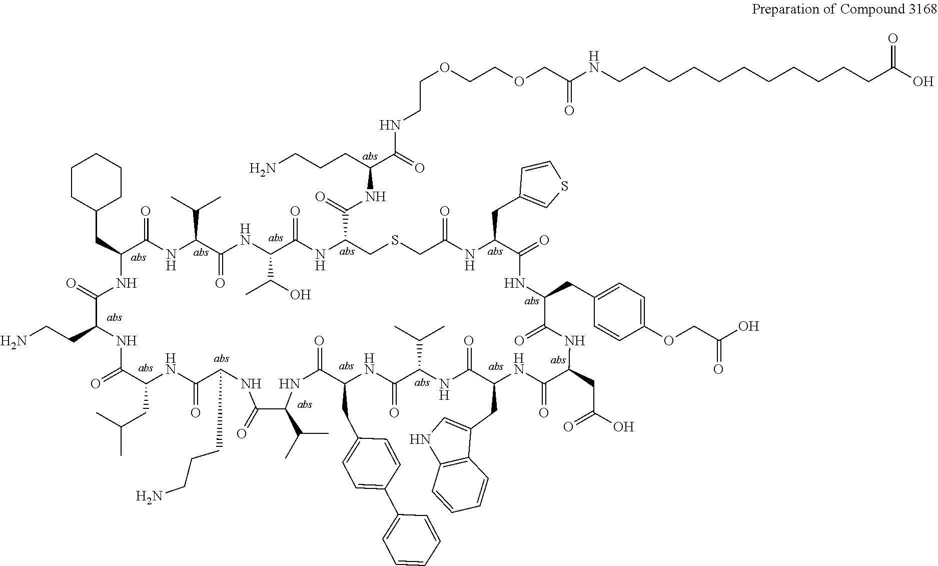 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@@H](CCCN)C(=O)NCCOCCOCC(=O)NCCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3168 was prepared on a 50 μmol scale. The yield of the product was 27.4 mg, and its estimated purity by LCMS analysis was 95.3%. Analysis condition A: Retention time=1.81 min; ESI-MS(+) m/z [M+2H]²⁺: 1199.

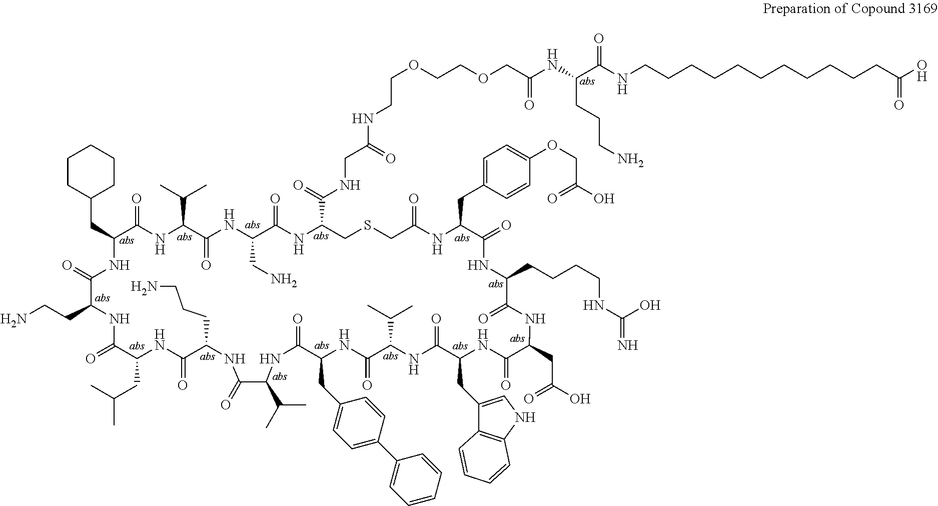 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCCCNC(=N)O)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3169 was prepared on a 50 μmol scale. The yield of the product was 33.3 mg, and its estimated purity by LCMS analysis was 100%. Analysis condition A: Retention time=1.59 min; ESI-MS(+) m/z [M+3H]³⁺: 818.9.

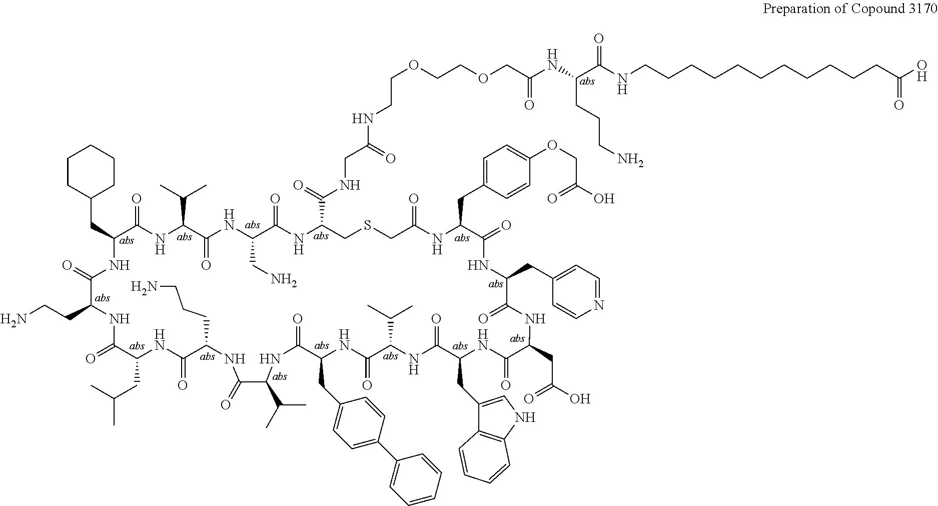 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3170 was prepared on a 50 μmol scale. The yield of the product was 24.8 mg, and its estimated purity by LCMS analysis was 100%. Analysis condition A: Retention time=1.8 min; ESI-MS(+) m/z [M+2H]²⁺: 1216.8.

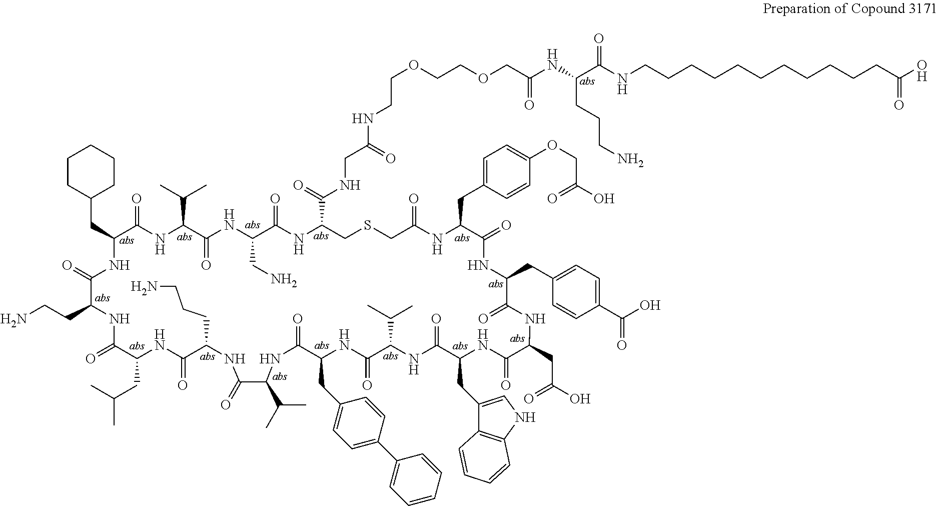 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3171 was prepared on a 50 μmol scale. The yield of the product was 41.1 mg, and its estimated purity by LCMS analysis was 98.3%. Analysis condition A: Retention time=1.68 min; ESI-MS(+) m/z [M+3H]³⁺: 826.2.

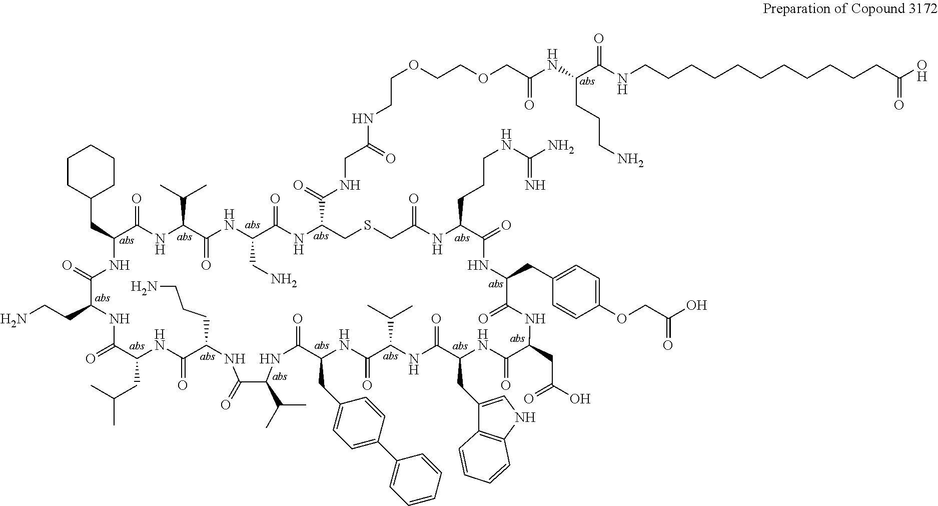 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3172 was prepared on a 50 μmol scale. The yield of the product was 27.5 mg, and its estimated purity by LCMS analysis was 100%. Analysis condition A: Retention time=1.59 min; ESI-MS(+) m/z [M+2H]²⁺: 1221.1.

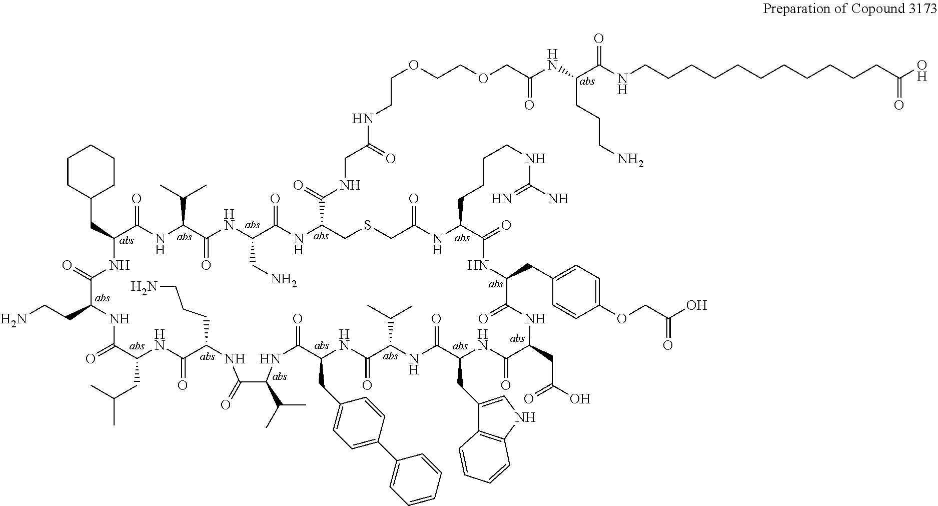 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3173 was prepared on a 50 μmol scale. The yield of the product was 10.6 mg, and its estimated purity by LCMS analysis was 96.8%. Analysis condition B: Retention time=1.45 min; ESI-MS(+) m/z [M+3H]³⁺: 819.2.

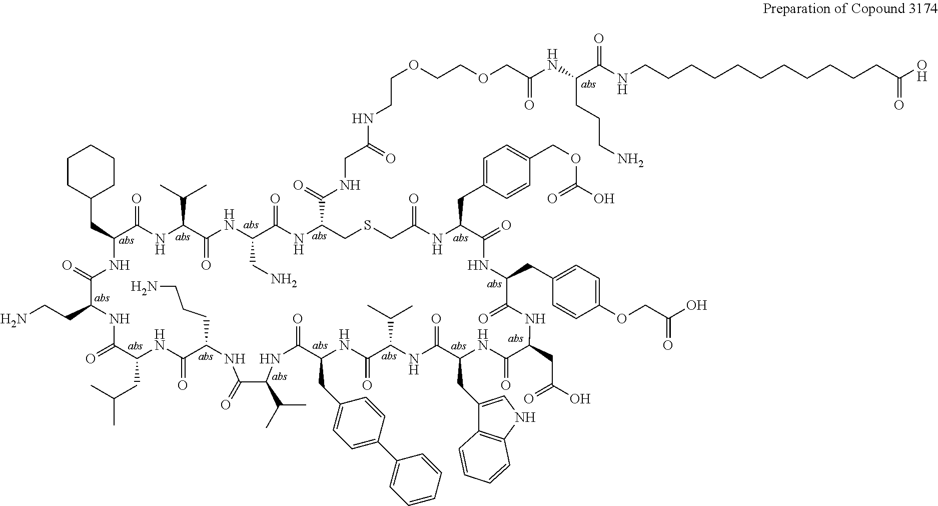 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(COC(=O)O)cc2)NC(=O)CSC[C@@H](C(=O)NCC(=O)NCCOCCOCC(=O)N[C@@H](CCCN)C(=O)NCCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3174 was prepared on a 50 μmol scale. The yield of the product was 22.3 mg, and its estimated purity by LCMS analysis was 97%. Analysis condition B: Retention time=1.6 min; ESI-MS(+) m/z [M+2H]²⁺: 1253.9.

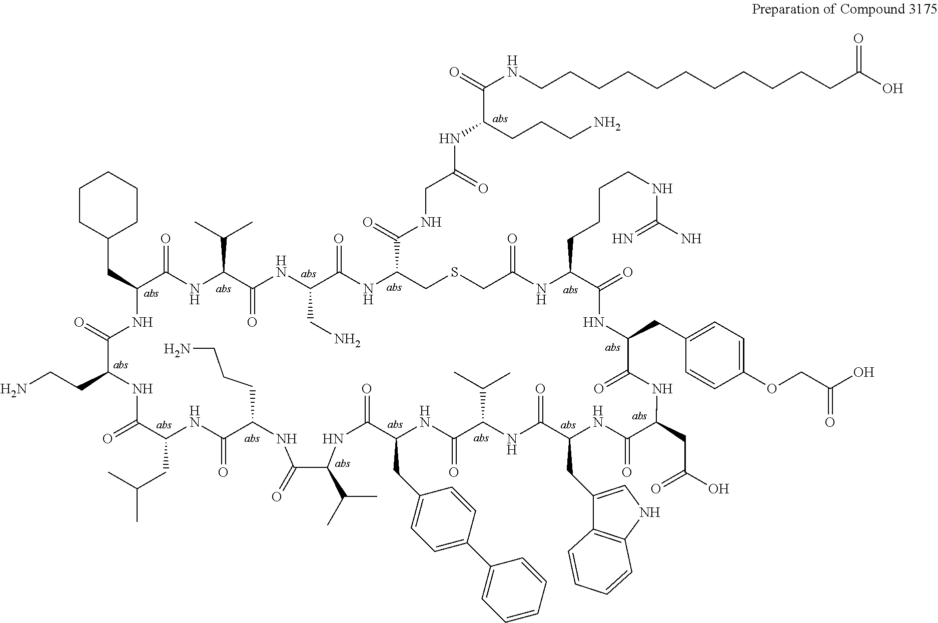 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)N[C@@H](CCCN)C(=O)NCCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3175 was prepared on a 50 μmol scale. The yield of the product was 9.3 mg, and its estimated purity by LCMS analysis was 94.1%. Analysis condition A: Retention time-1.68 min; ESI-MS(+) m/z [M+2H]²⁺: 1156.1.

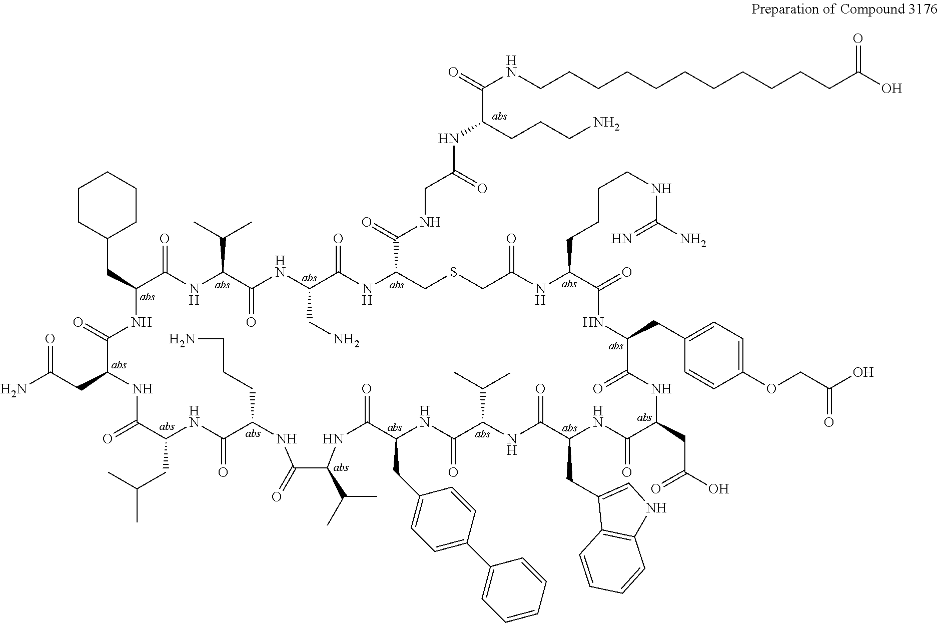 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)N[C@@H](CCCN)C(=O)NCCCCCCCCCCCC(=O)O)NC(=O)[C@H](CN)NC(=O)[C@H](C(C)C)NC(=O)[C@H](CC2CCCCC2)NC(=O)[C@H](CC(N)=O)NC1=O]

Compound 3176 was prepared on a 50 μmol scale. The yield of the product was 32.9 mg, and its estimated purity by LCMS analysis was 97.7%. Analysis condition B: Retention time=1.63 min; ESI-MS(+) m/z [M+3H]³⁺: 775.1.

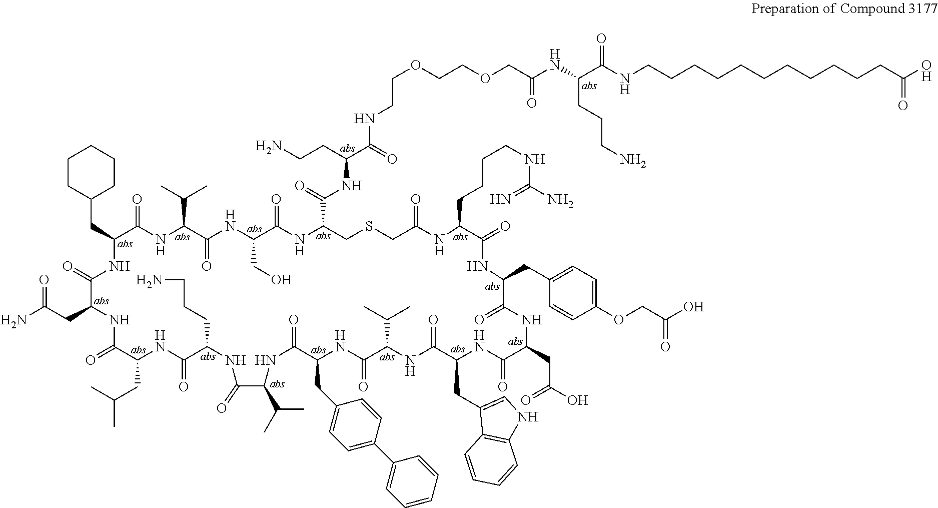 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC1=O]

Compound 3177 was prepared on a 50 μmol scale. The yield of the product was 3.5 mg, and its estimated purity by LCMS analysis was 100%. Analysis condition A: Retention time=1.54 min; ESI-MS(+) m/z [M+3H]³⁺: 838.2.

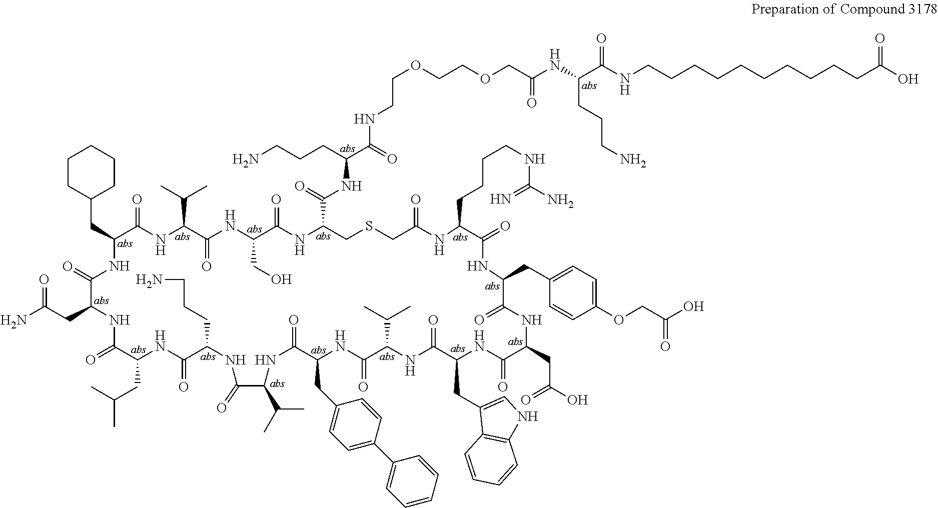 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCCN)C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CC(N)=O)NC1=O]

Compound 3178 was prepared on a 50 μmol scale. The yield of the product was 27.5 mg, and its estimated purity by LCMS analysis was 98%. Analysis condition A: Retention time=1.59 min; ESI-MS(+) m/z [M+2H]²⁺: 1257.1.

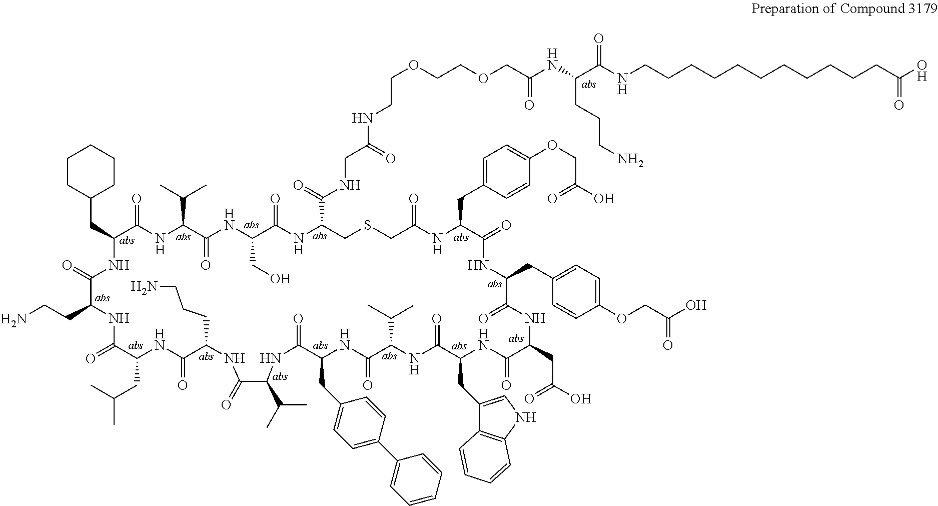 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3179 was prepared on a 50 μmol scale. The yield of the product was 26.1 mg, and its estimated purity by LCMS analysis was 96.3%. Analysis condition B: Retention time=1.66 min; ESI-MS(+) m/z [M+2H]²⁺: 1254.3.

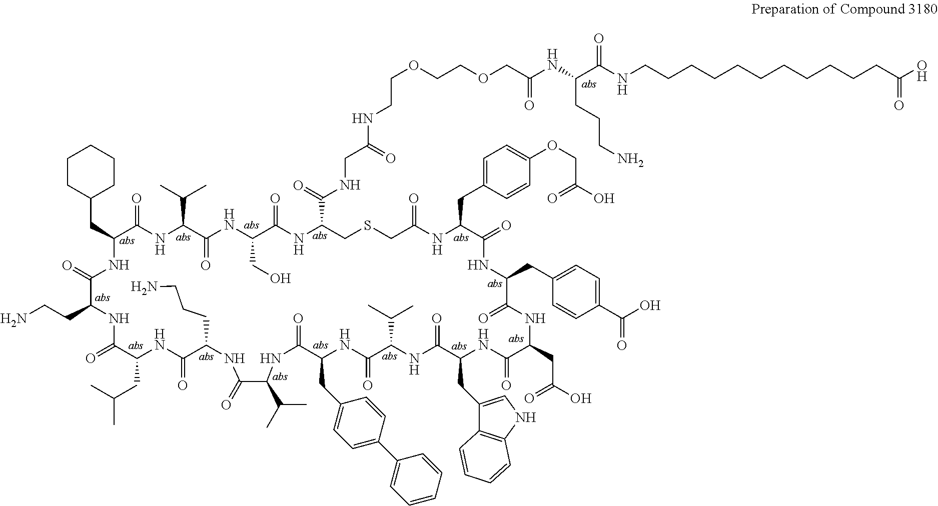 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3180 was prepared on a 50 μmol scale. The yield of the product was 41.2 mg, and its estimated purity by LCMS analysis was 95%. Analysis condition A: Retention time=1.69 min; ESI-MS(+) m/z [M+2H]²⁺: 1239.2.

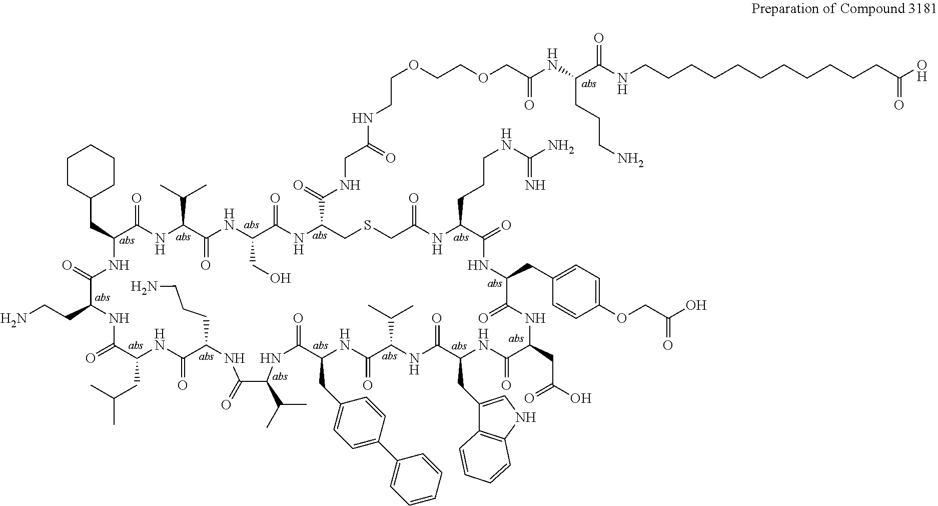 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3181 was prepared on a 50 μmol scale. The yield of the product was 13.6 mg, and its estimated purity by LCMS analysis was 96.5%. Analysis condition B: Retention time=1.53 min; ESI-MS(+) m/z [M+3H]³⁺: 815.

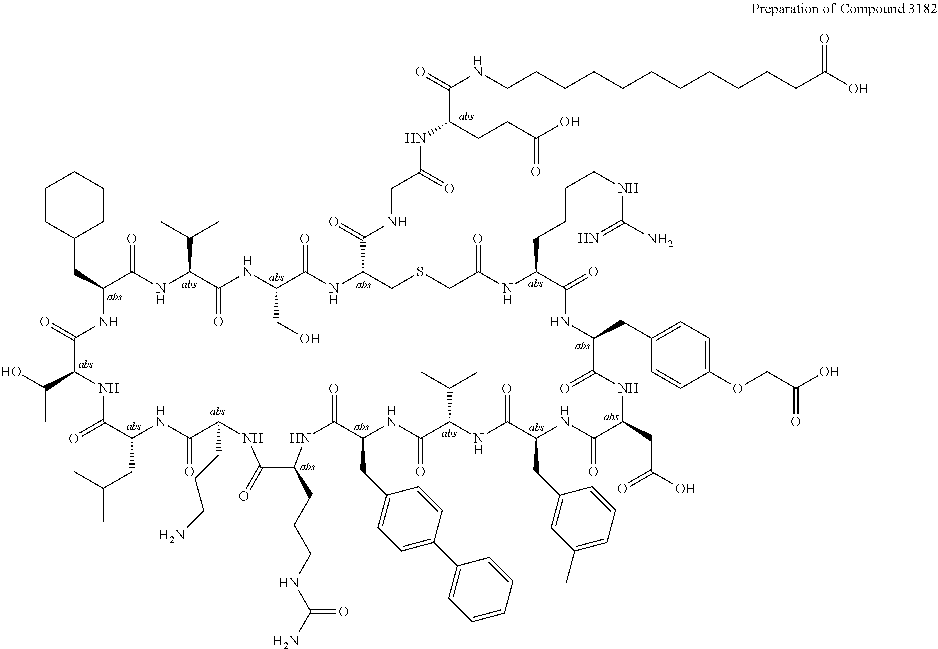 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCC(=O)N[C@@H](CCC(=O)O)C(=O)NCCCCCCCCCCCC(=O)O)NC(=O)[C@H](CO)NC(=O)[C@H](C(C)C)NC(=O)[C@H](CC3CCCCC3)NC(=O)[C@H](C(C)O)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3182 was prepared on a 50 μmol scale. The yield of the product was 8.4 mg, and its estimated purity by LCMS analysis was 95%. Analysis condition B: Retention time=1.78 min; ESI-MS(+) m/z [M+3H]³⁺: 787.

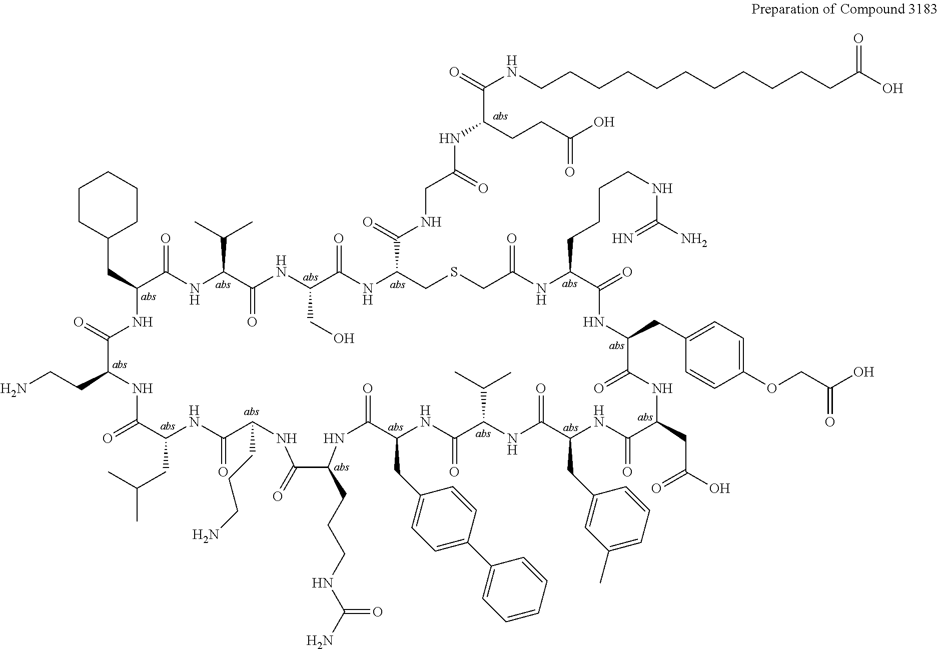 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCC(=O)N[C@@H](CCC(=O)O)C(=O)NCCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3183 was prepared on a 50 μmol scale. The yield of the product was 0.9 mg, and its estimated purity by LCMS analysis was 100%. Analysis condition A: Retention time=1.61 min; ESI-MS(+) m/z [M+2H]²⁺: 1180.1.

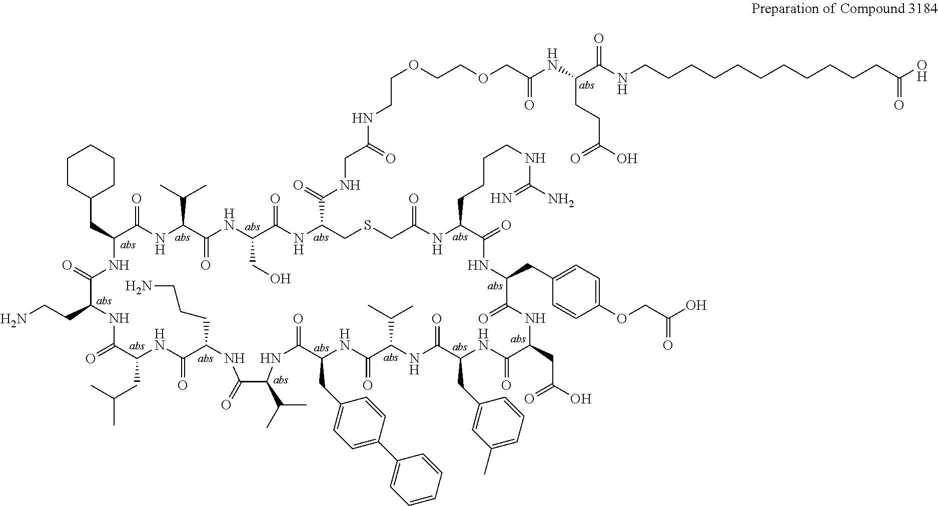 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCC(=O)NCCOCCOCC(=O)N[C@@H](CCC(=O)O)C(=O)NCCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3184 was prepared on a 50 μmol scale. The yield of the product was 15.9 mg, and its estimated purity by LCMS analysis was 100%. Analysis condition A: Retention time=1.65 min; ESI-MS(+) m/z [M+3H]³⁺: 816.3.

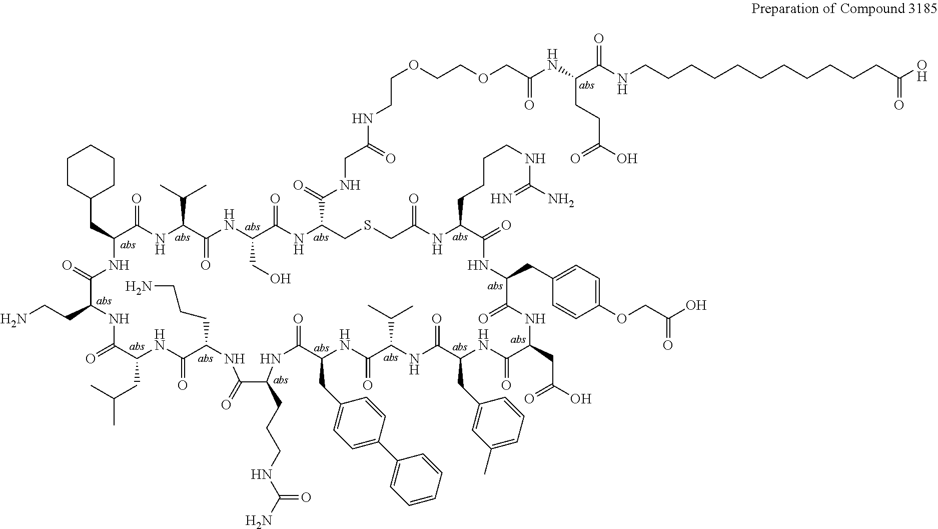 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCC(=O)NCCOCCOCC(=O)N[C@@H](CCC(=O)O)C(=O)NCCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3185 was prepared on a 50 μmol scale. The yield of the product was 14.6 mg, and its estimated purity by LCMS analysis was 95.7%. Analysis condition B: Retention time=1.65 min; ESI-MS(+) m/z [M+3H]³⁺: 835.1.

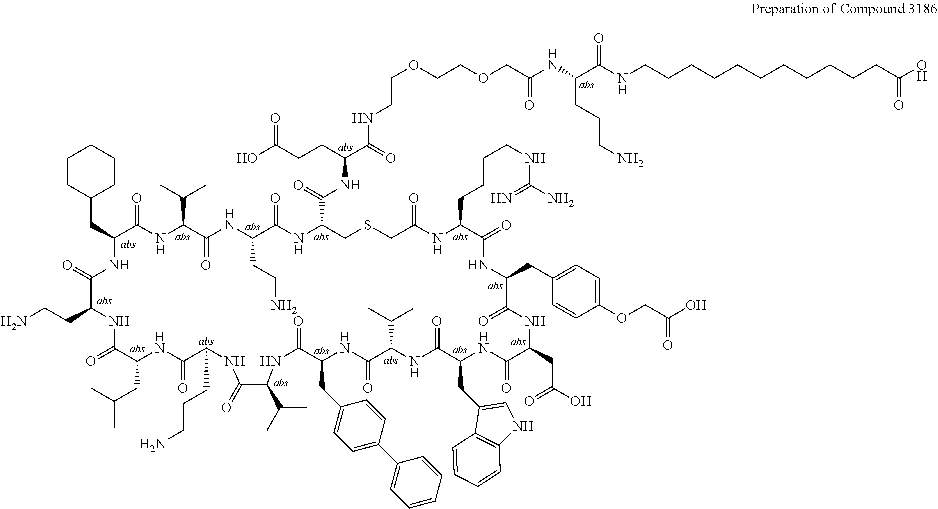 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCC(=O)O)C(=O)NCCOCCOCC(=O)N[C@@H](CCCN)C(=O)NCCCCCCCCCCCC(=O)O)NC(=O)[C@H](CCN)NC(=O)[C@H](C(C)C)NC(=O)[C@H](CC2CCCCC2)NC(=O)[C@H](CCN)NC1=O]

Compound 3186 was prepared on a 50 μmol scale. The yield of the product was 43.5 mg, and its estimated purity by LCMS analysis was 96%. Analysis condition B: Retention time=1.52 min; ESI-MS(+) m/z [M+2H]²⁺: 1278.3.

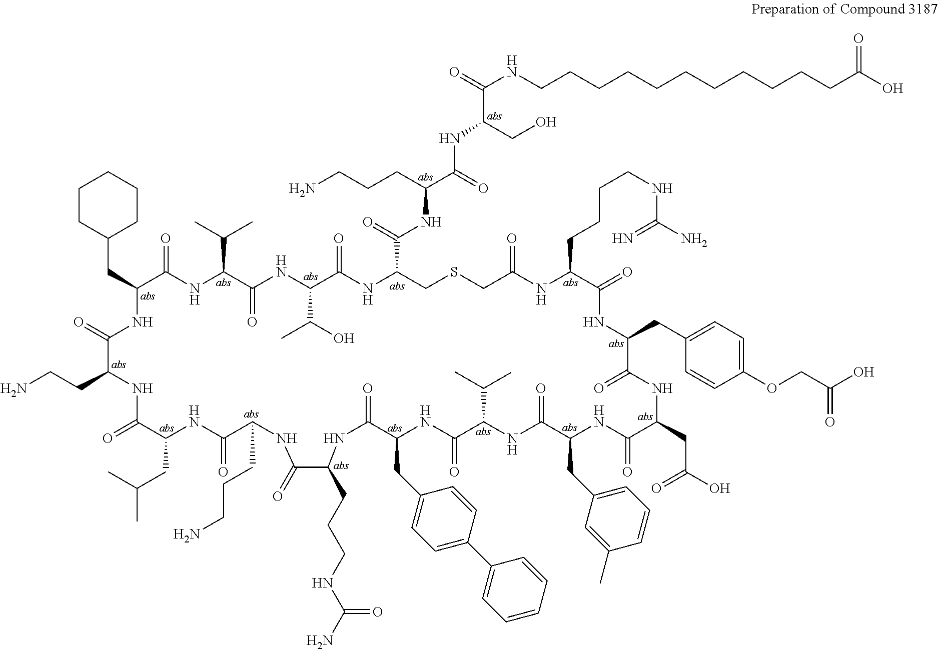 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3187 was prepared on a 50 μmol scale. The yield of the product was 9.1 mg, and its estimated purity by LCMS analysis was 97.7%. Analysis condition B: Retention time=1.57 min; ESI-MS(+) m/z [M+3H]³⁺: 797.

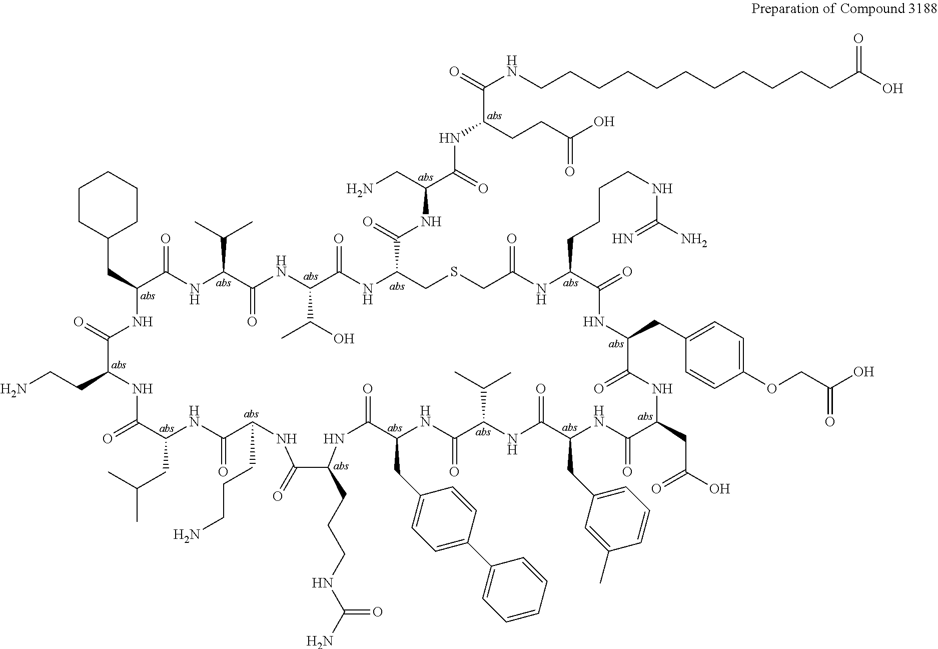 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CN)C(=O)N[C@@H](CCC(=O)O)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3188 was prepared on a 50 μmol scale. The yield of the product was 0.9 mg, and its estimated purity by LCMS analysis was 100%. Analysis condition A: Retention time=1.66 min; ESI-MS(+) m/z [M+3H]³⁺: 801.4.

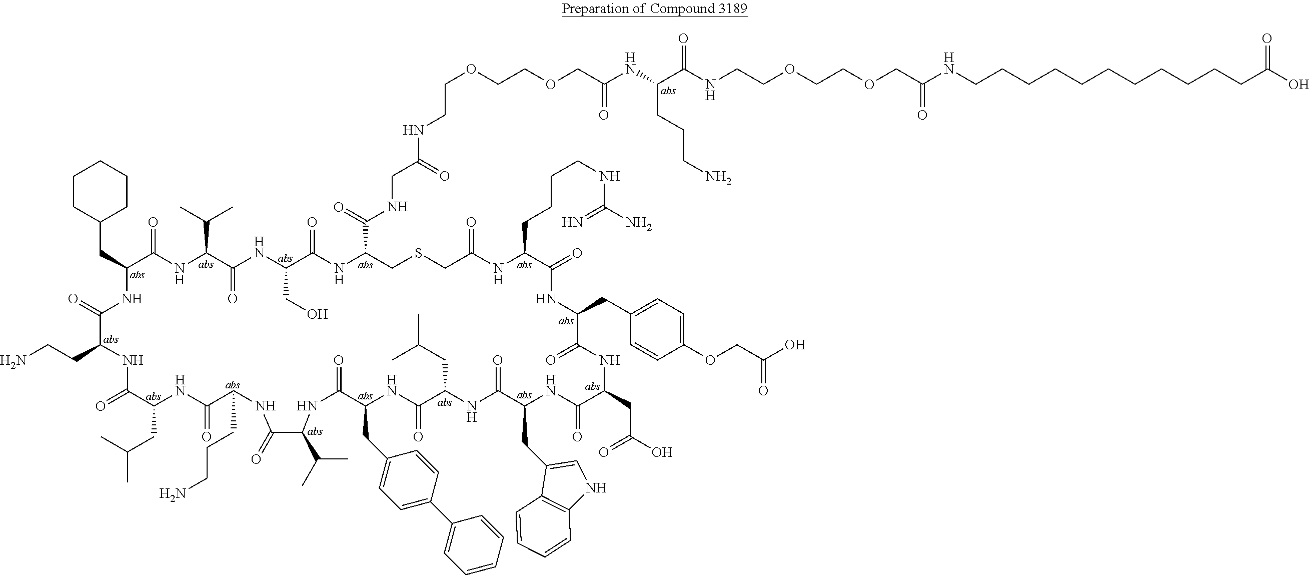 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCOCCOCC(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3189 was prepared on a 50 μmol scale. The yield of the product was 21.3 mg, and its estimated purity by LCMS analysis was 96%. Analysis condition B: Retention time=1.6 min; ESI-MS(+) m/z [M+2H]²⁺: 1308.2.

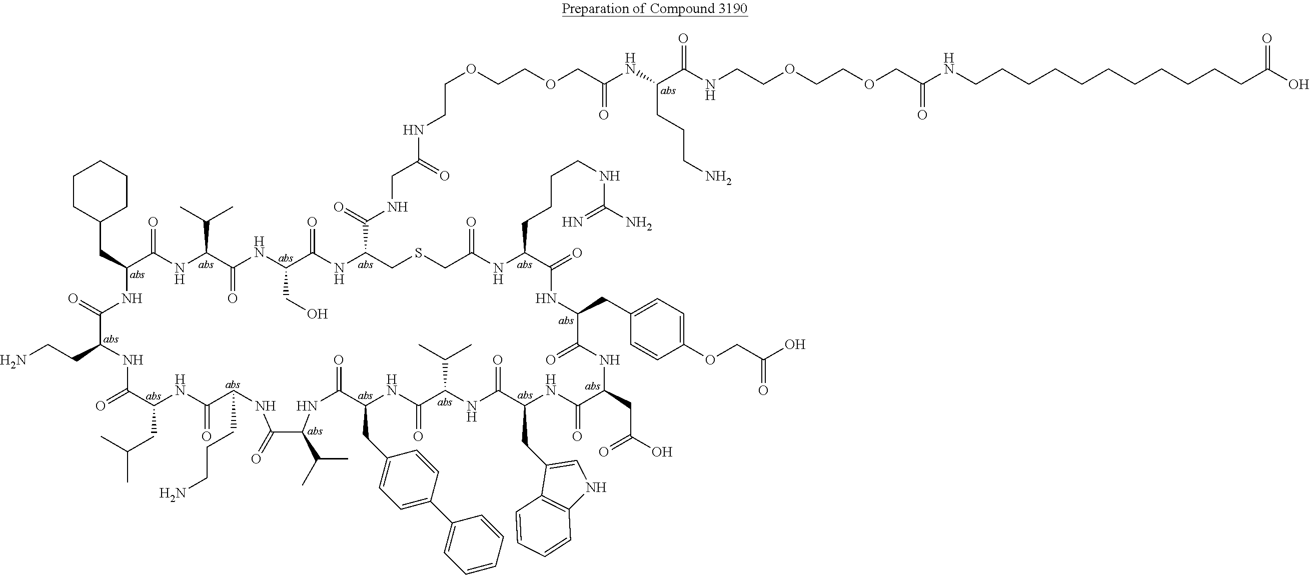 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCOCCOCC(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3190 was prepared on a 50 μmol scale. The yield of the product was 25.1 mg, and its estimated purity by LCMS analysis was 95.8%. Analysis condition A: Retention time=1.64 min; ESI-MS(+) m/z [M+3H]³⁺: 867.1.

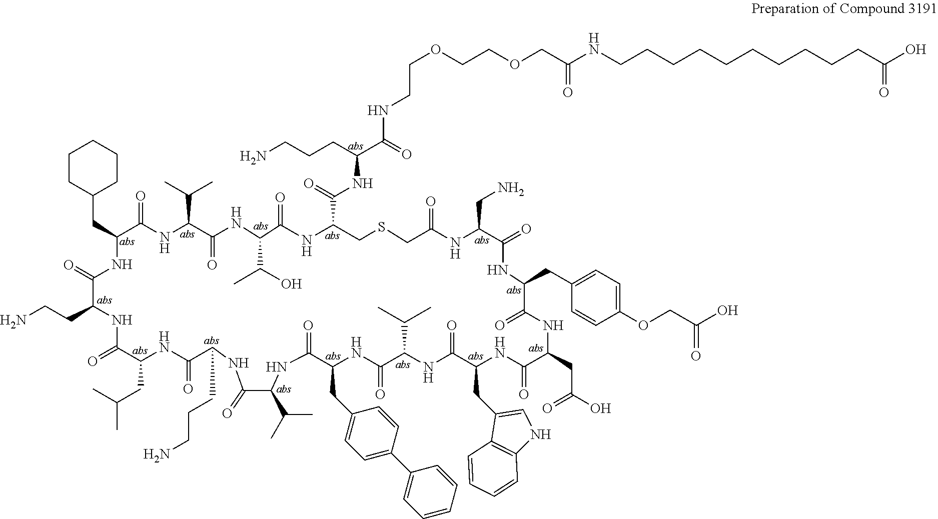 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3191 was prepared on a 50 μmol scale. The yield of the product was 25 mg, and its estimated purity by LCMS analysis was 95.8%. Analysis condition B: Retention time=1.49 min; ESI-MS(+) m/z [M+3H]³⁺: 772.1.

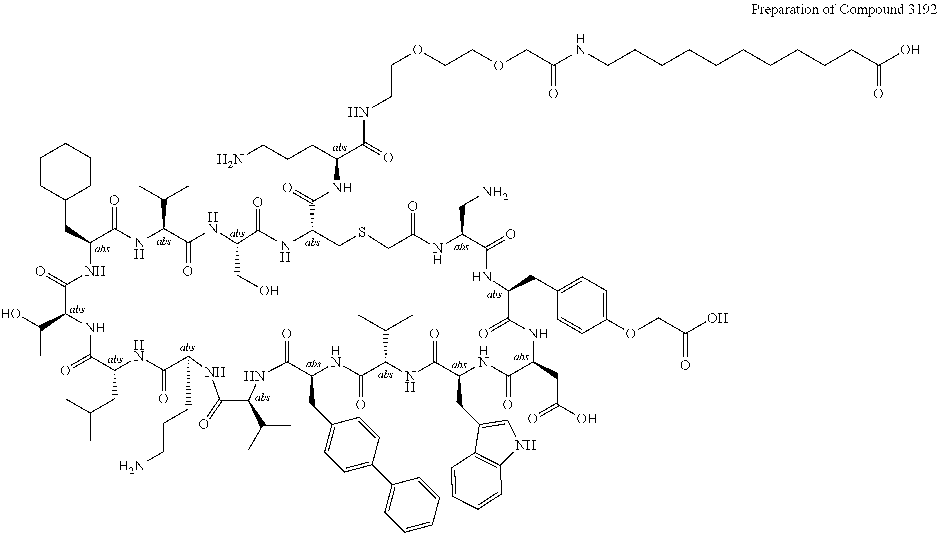 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCCN)C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](C(C)O)NC1=O]

Compound 3192 was prepared on a 50 μmol scale. The yield of the product was 7.4 mg, and its estimated purity by LCMS analysis was 89.4%. Analysis condition B: Retention time=1.68 min; ESI-MS(+) m/z [M+2H]²⁺: 1151.

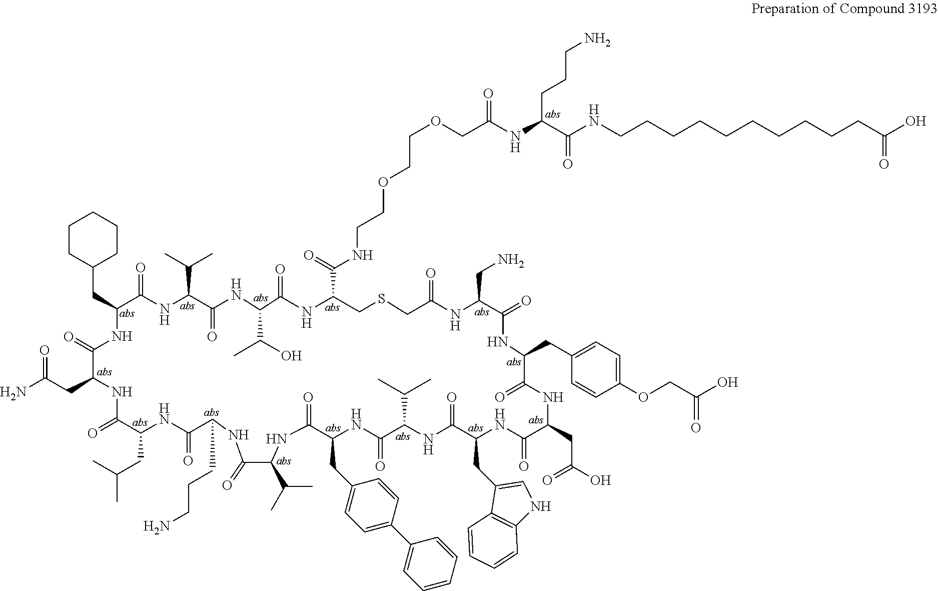 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CC(N)=O)NC1=O]

Compound 3193 was prepared on a 50 μmol scale. The yield of the product was 41 mg, and its estimated purity by LCMS analysis was 96.7%. Analysis condition A: Retention time=1.72 min; ESI-MS(+) m/z [M+2H]²⁺: 1165.2.

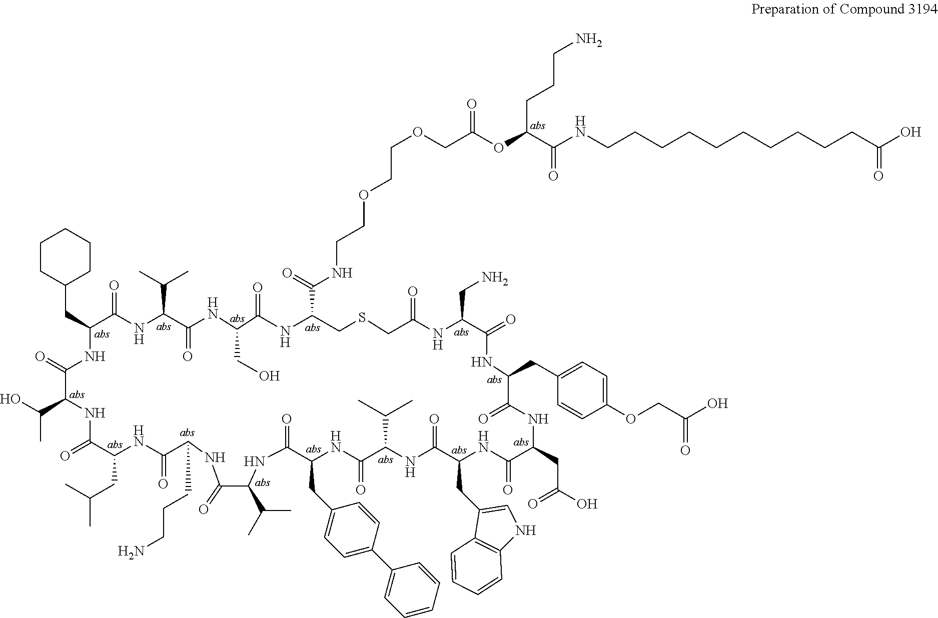 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)O[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](C(C)O)NC1=O]

Compound 3194 was prepared on a 50 μmol scale. The yield of the product was 10 mg, and its estimated purity by LCMS analysis was 90.3%. Analysis condition B: Retention time=1.69 min; ESI-MS(+) m/z [M+3H]³⁺: 768.

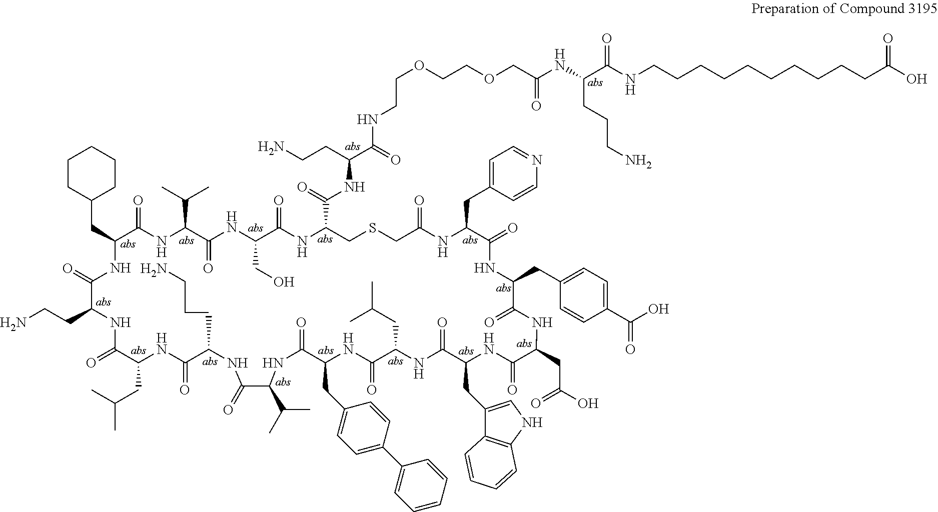 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCN)C(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3195 was prepared on a 50 μmol scale. The yield of the product was 17.2 mg, and its estimated purity by LCMS analysis was 97.4%. Analysis condition B: Retention time=1.49 min; ESI-MS(+) m/z [M+3H]³⁺: 816.4.

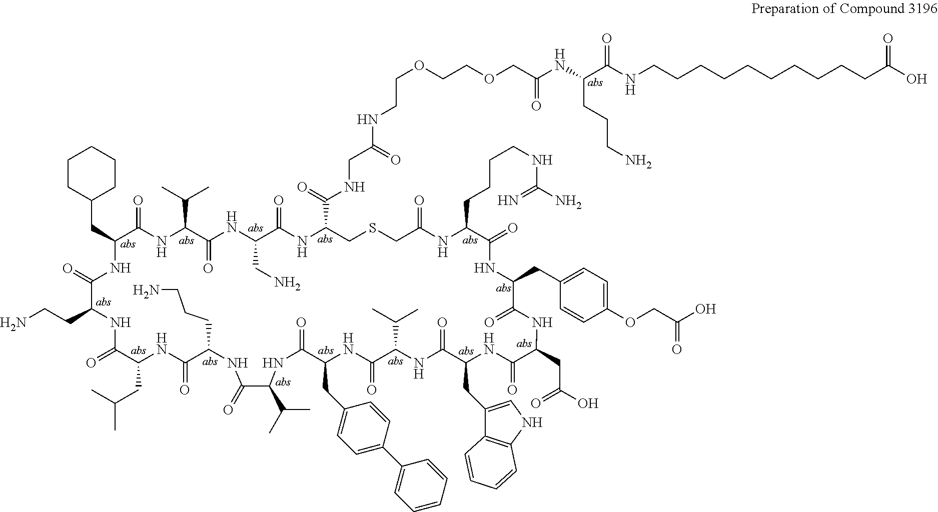 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3196 was prepared on a 50 μmol scale. The yield of the product was 48.5 mg, and its estimated purity by LCMS analysis was 97.2%. Analysis condition A: Retention time=1.6 min; ESI-MS(+) m/z [M+2H]²⁺: 1221.1.

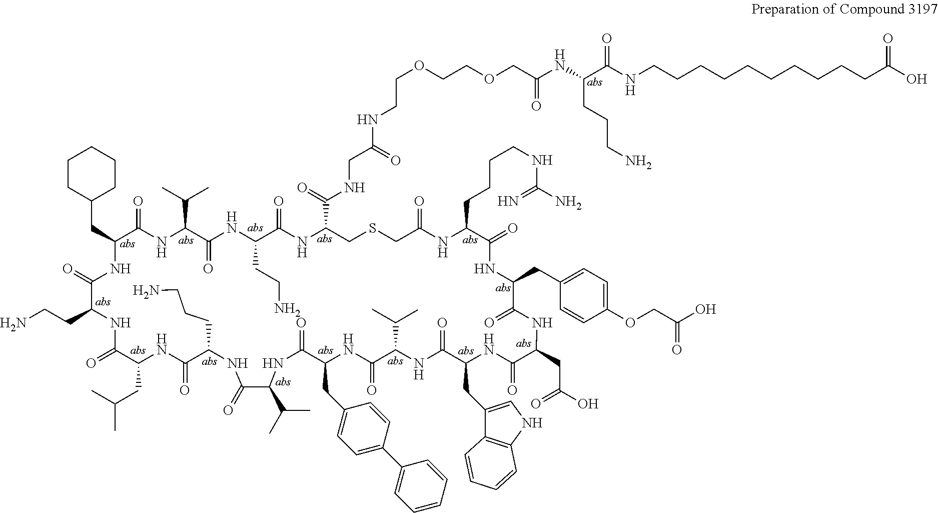 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCN)NC(=O)[C@H](C(C)C)NC(=O)[C@H](CC2CCCCC2)NC(=O)[C@H](CCN)NC1=O]

Compound 3197 was prepared on a 50 μmol scale. The yield of the product was 44.9 mg, and its estimated purity by LCMS analysis was 99%. Analysis condition A: Retention time=1.51 min; ESI-MS(+) m/z [M+3H]³⁺: 819.1.

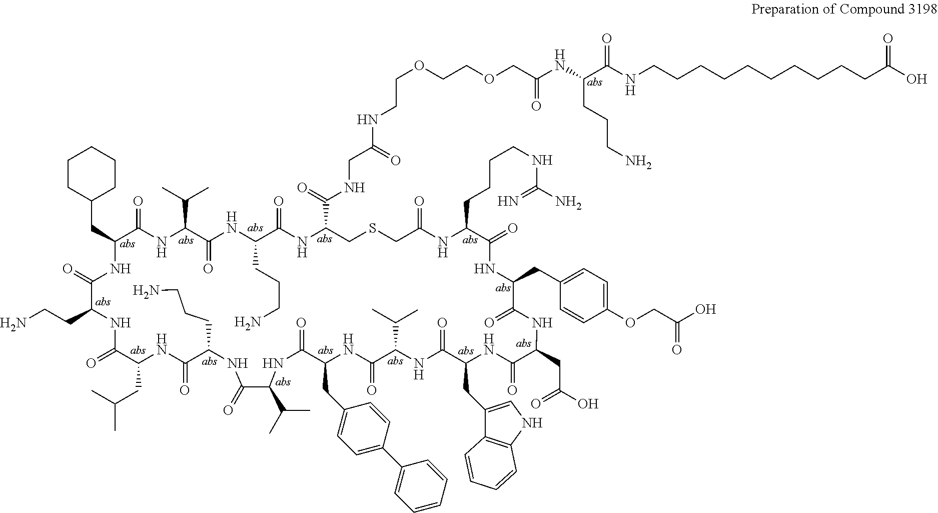 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 3198 was prepared on a 50 μmol scale. The yield of the product was 33.4 mg, and its estimated purity by LCMS analysis was 96.6%. Analysis condition A: Retention time=1.53 min; ESI-MS(+) m/z [M+2H]²⁺: 1235.1.

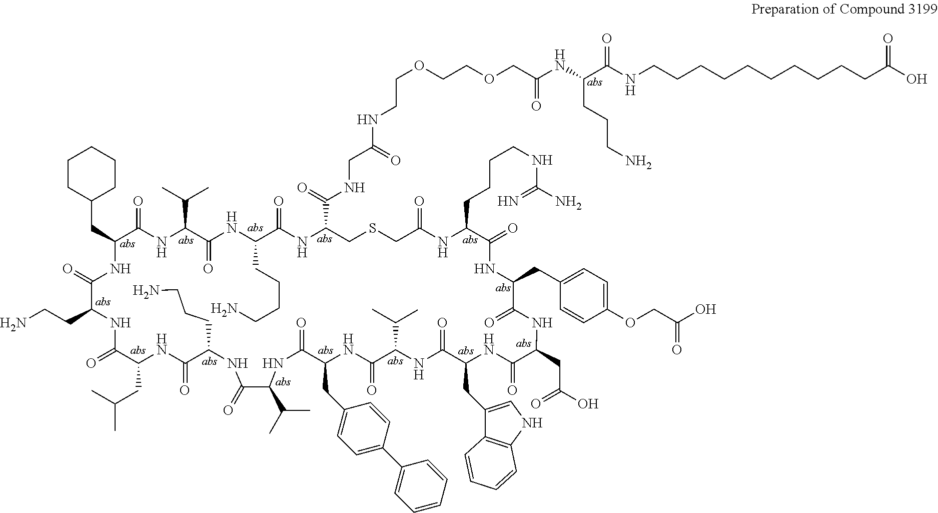 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 3199 was prepared on a 50 μmol scale. The yield of the product was 28.1 mg, and its estimated purity by LCMS analysis was 98%. Analysis condition A: Retention time-1.54 min; ESI-MS(+) m/z [M+3H]³⁺: 828.1.

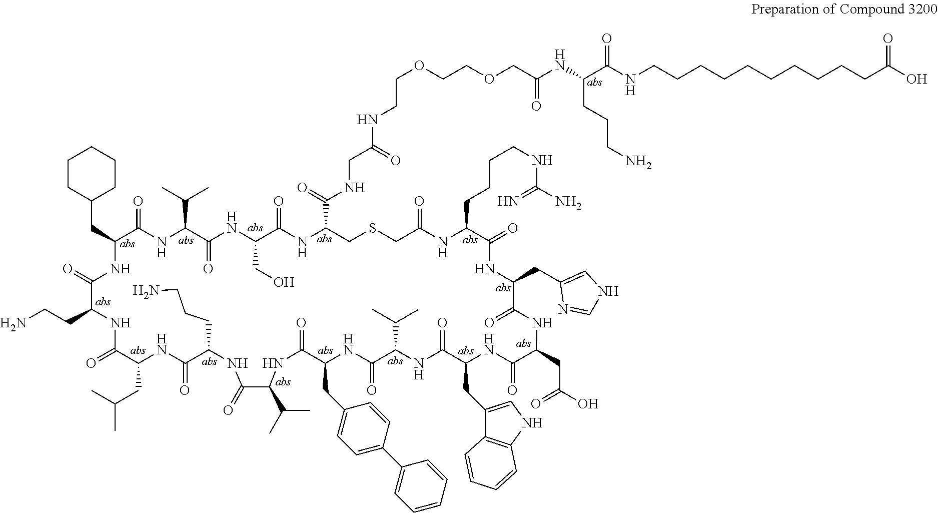 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]cn2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3200 was prepared on a 50 μmol scale. The yield of the product was 22.4 mg, and its estimated purity by LCMS analysis was 96.6%. Analysis condition A: Retention time=1.6 min; ESI-MS(+) m/z [M+3H]³⁺: 787.1.

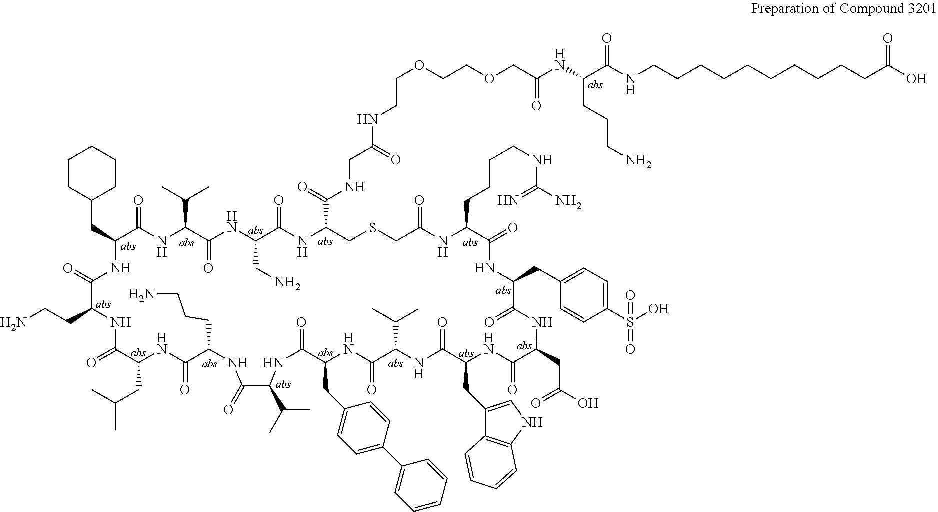 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(S(=O)(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3201 was prepared on a 50 μmol scale. The yield of the product was 76.4 mg, and its estimated purity by LCMS analysis was 97.2%. Analysis condition B: Retention time=1.6 min; ESI-MS(+) m/z [M+2H]²⁺: 1231.

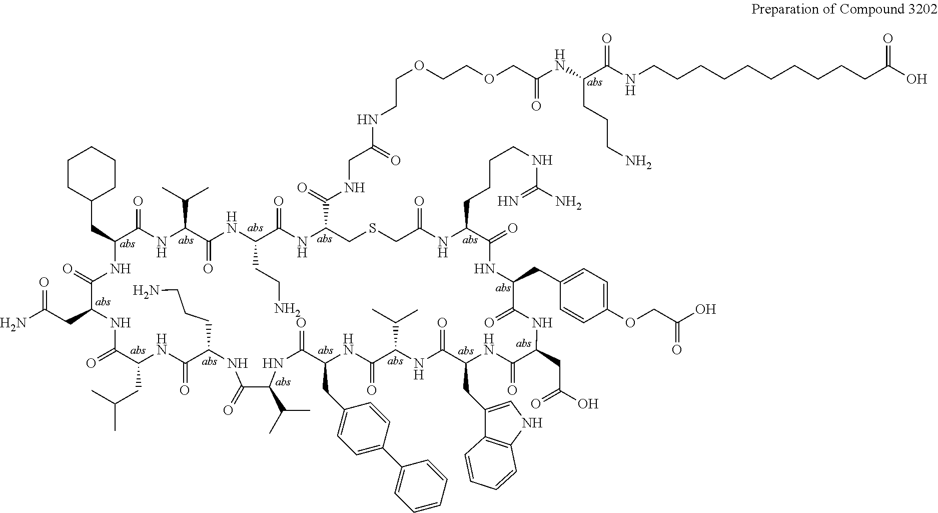 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCN)NC(=O)[C@H](C(C)C)NC(=O)[C@H](CC2CCCCC2)NC(=O)[C@H](CC(N)=O)NC1=O]

Compound 3202 was prepared on a 50 μmol scale. The yield of the product was 64.3 mg, and its estimated purity by LCMS analysis was 95.7%. Analysis condition A: Retention time=1.57 min; ESI-MS(+) m/z [M+2H]²⁺: 1235.1.

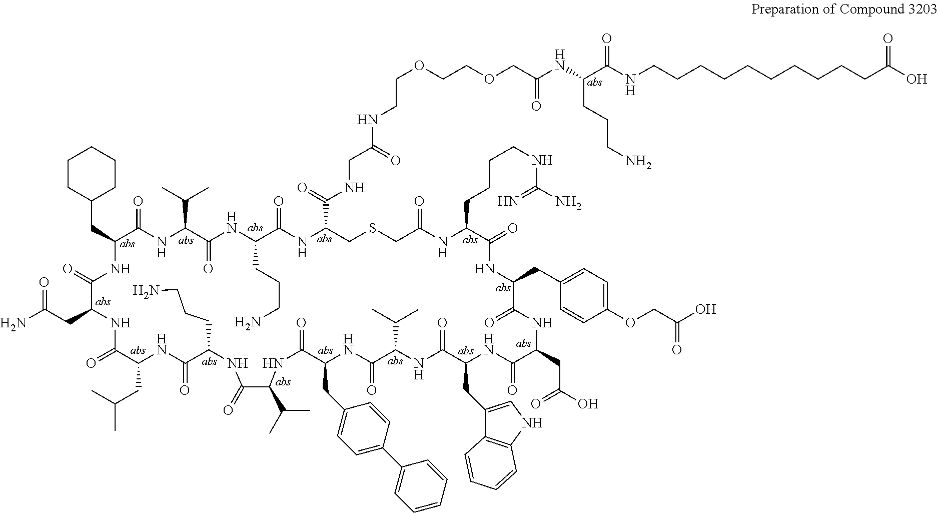 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC1=O]

Compound 3203 was prepared on a 50 μmol scale. The yield of the product was 16.2 mg, and its estimated purity by LCMS analysis was 92.4%. Analysis condition B: Retention time=1.5 min; ESI-MS(+) m/z [M+3H]³⁺: 829.1.

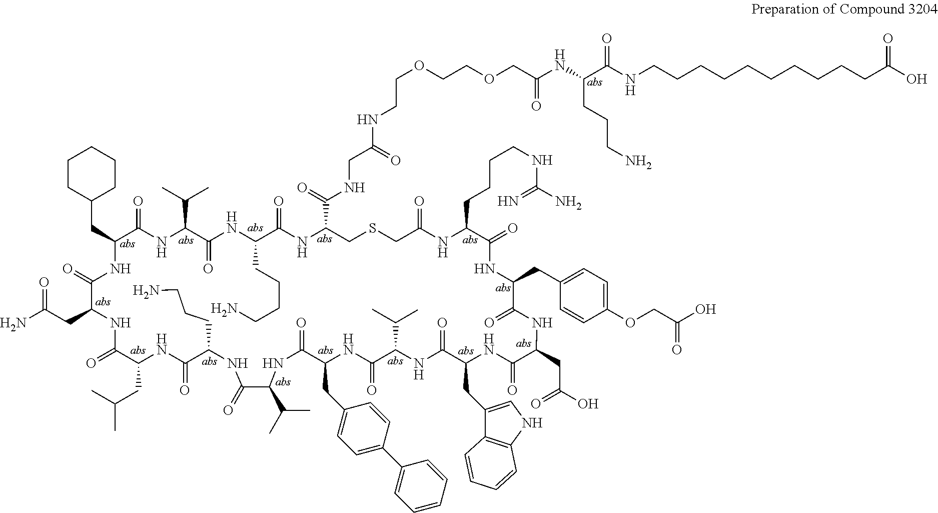 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CC(N)=O)NC1=O]

Compound 3204 was prepared on a 50 μmol scale. The yield of the product was 11.9 mg, and its estimated purity by LCMS analysis was 99%. Analysis condition B: Retention time=1.48 min; ESI-MS(+) m/z [M+3H]³⁺: 833.

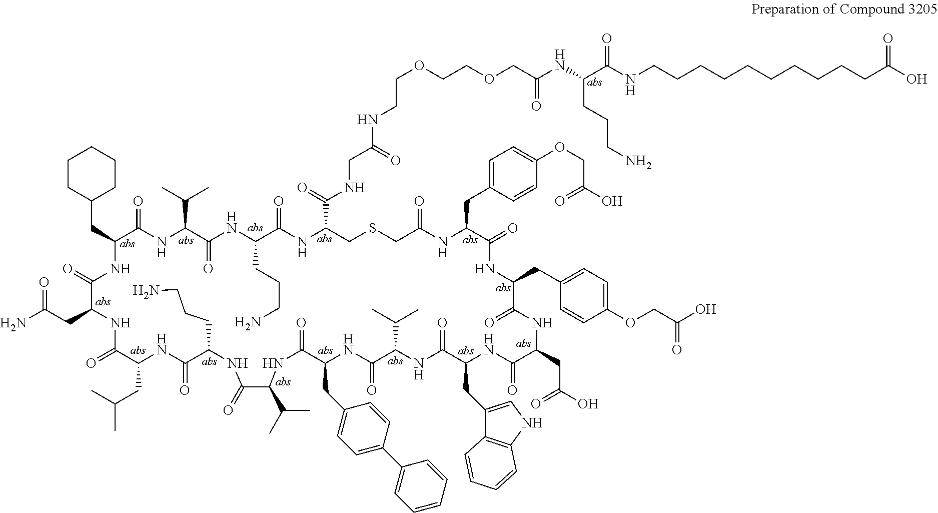 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC1=O]

Compound 3205 was prepared on a 50 μmol scale. The yield of the product was 54.9 mg, and its estimated purity by LCMS analysis was 98.6%. Analysis condition B: Retention time=1.67 min; ESI-MS(+) m/z [M+3H]³⁺: 845.1.

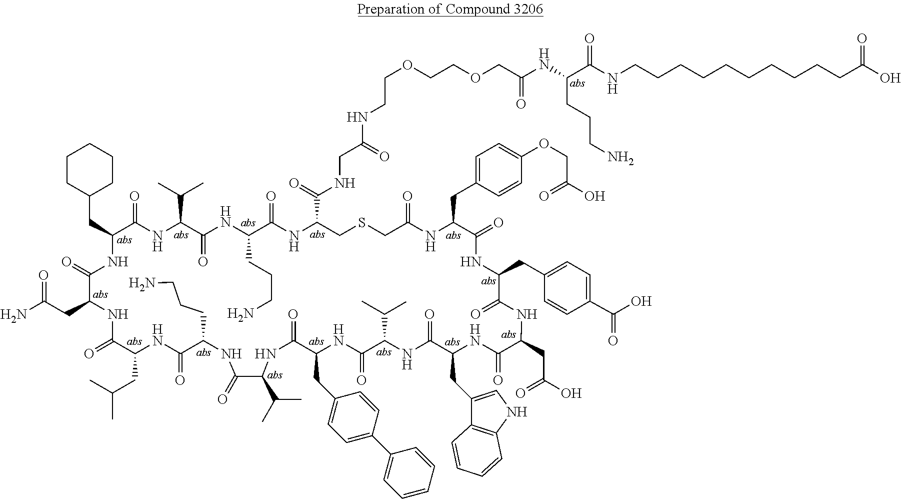 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC1=O]

Compound 3206 was prepared on a 50 μmol scale. The yield of the product was 46.3 mg, and its estimated purity by LCMS analysis was 95.1%. Analysis condition A: Retention time=1.55 min; ESI-MS(+) m/z [M+2H]²⁺: 1253.

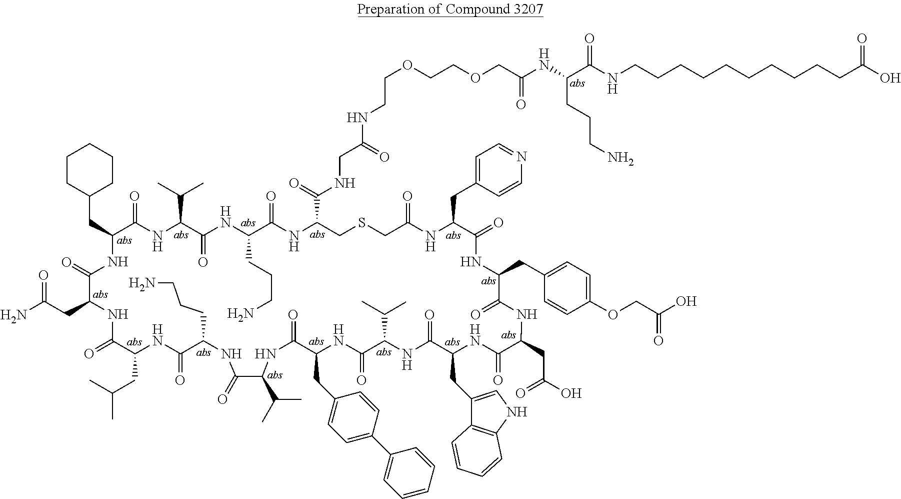 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC1=O]

Compound 3207 was prepared on a 50 μmol scale. The yield of the product was 21.5 mg, and its estimated purity by LCMS analysis was 95.6%. Analysis condition A: Retention time=1.58, 1.62 min; ESI-MS(+) m/z [M+2H]²⁺: 1231, 1231.

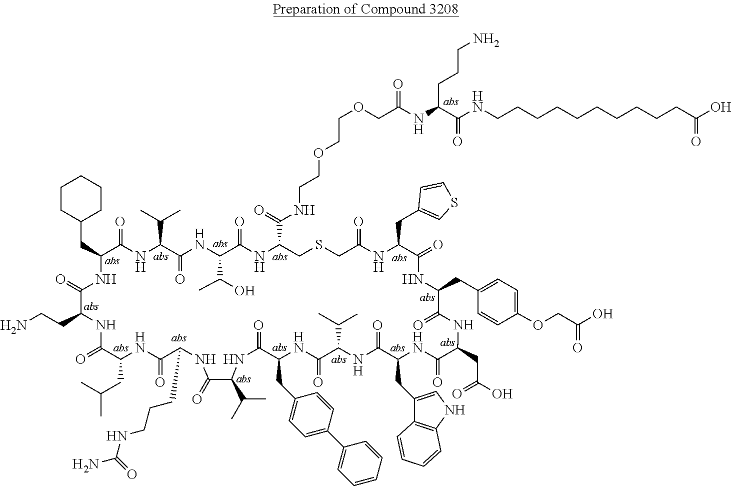 [SMILES: CC(C)C[C@H]1NC(=O)[C@H](CCCNC(N)=O)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3208 was prepared on a 50 μmol scale. The yield of the product was 25.1 mg, and its estimated purity by LCMS analysis was 84.9%. Analysis condition A: Retention time=1.93 min; ESI-MS(+) m/z [M+2H]²⁺: 1213.3.

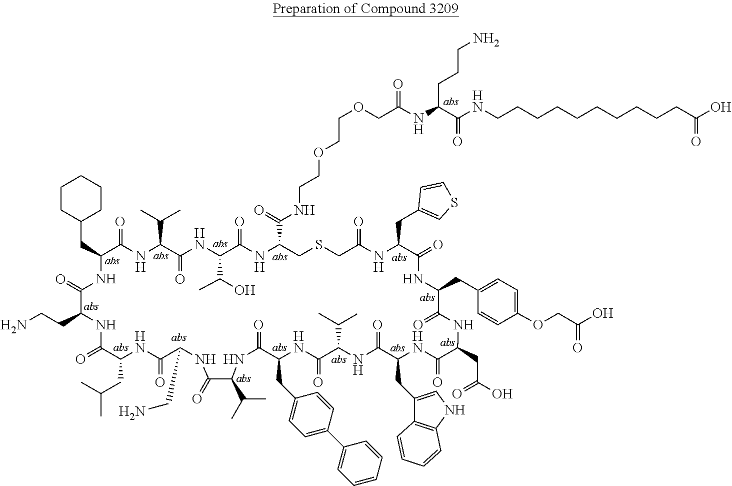 [SMILES: CC(C)C[C@H]1NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3209 was prepared on a 50 μmol scale. The yield of the product was 34.5 mg, and its estimated purity by LCMS analysis was 95.4%. Analysis condition B: Retention time=1.67 min; ESI-MS(+) m/z [M+2H]²⁺: 1178.

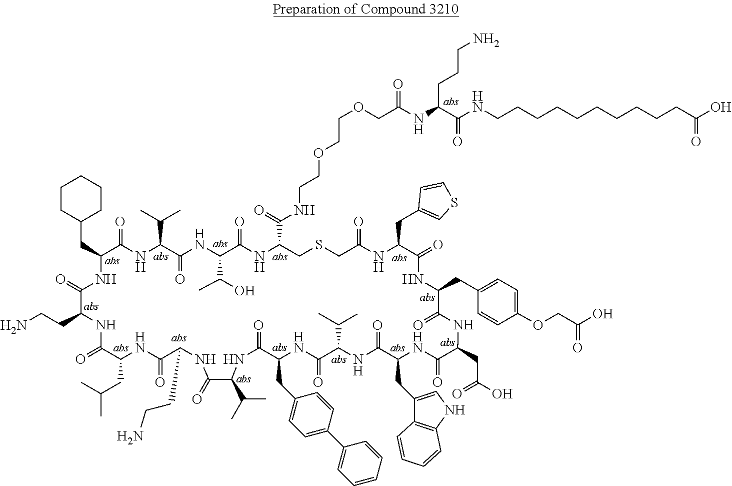 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3210 was prepared on a 50 μmol scale. The yield of the product was 31.1 mg, and its estimated purity by LCMS analysis was 96.3%. Analysis condition B: Retention time=1.67, 1.72 min; ESI-MS(+) m/z [M+3H]³⁺: 790.03, 790.03.

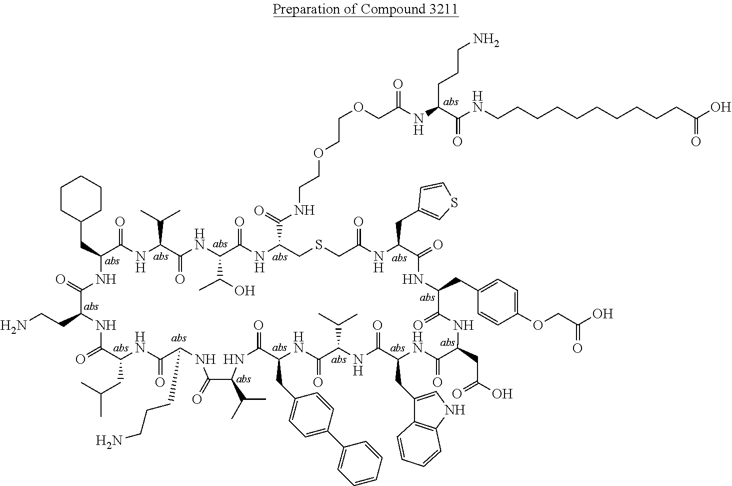 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3211 was prepared on a 50 μmol scale. The yield of the product was 28.1 mg, and its estimated purity by LCMS analysis was 95.9%. Analysis condition B: Retention time=1.67 min; ESI-MS(+) m/z [M+3H]³⁺: 794.2.

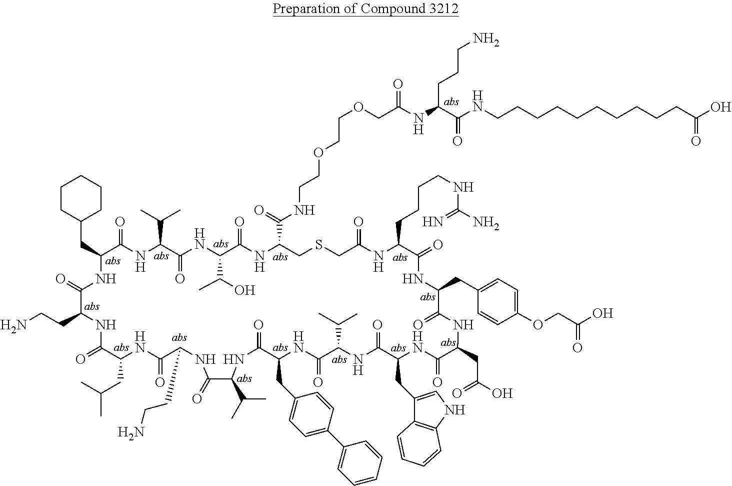 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3212 was prepared on a 50 μmol scale. The yield of the product was 13.4 mg, and its estimated purity by LCMS analysis was 95.9%. Analysis condition A: Retention time=1.65 min; ESI-MS(+) m/z [M+2H]²⁺: 1193.2.

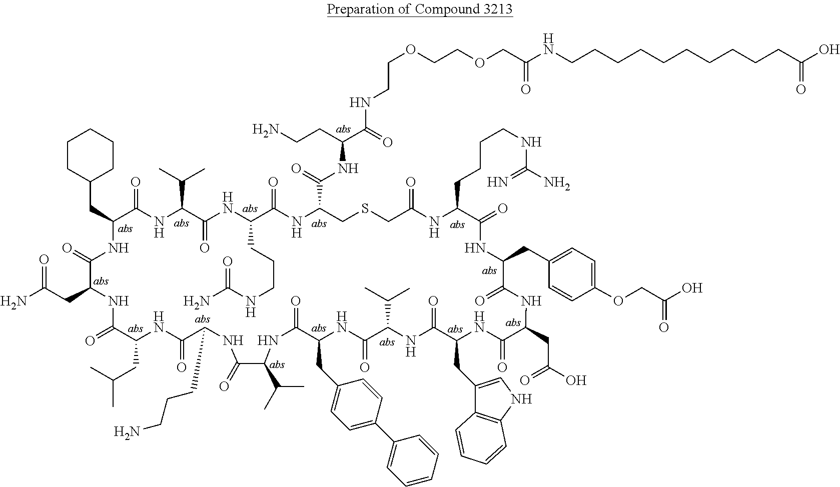 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CC(N)=O)NC1=O]

Compound 3213 was prepared on a 50 μmol scale. The yield of the product was 27.7 mg, and its estimated purity by LCMS analysis was 100%. Analysis condition A: Retention time=1.88 min; ESI-MS(+) m/z [M+3H]³⁺: 819.

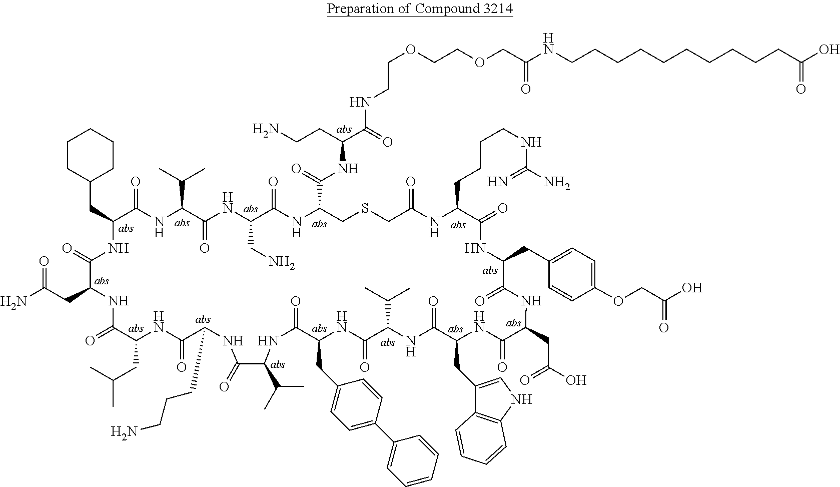 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CN)NC(=O)[C@H](C(C)C)NC(=O)[C@H](CC2CCCCC2)NC(=O)[C@H](CC(N)=O)NC1=O]

Compound 3214 was prepared on a 50 μmol scale. The yield of the product was 17 mg, and its estimated purity by LCMS analysis was 96.6%. Analysis condition A: Retention time=1.67 min; ESI-MS(+) m/z [M+2H]²⁺: 1193.2.

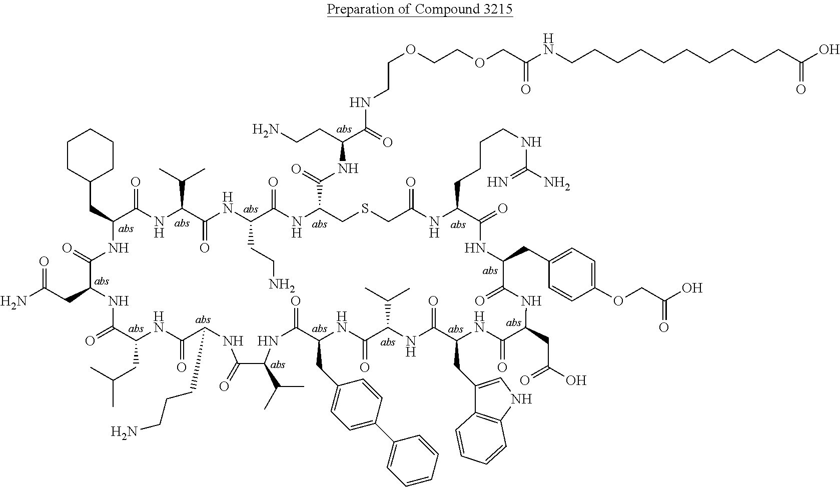 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CCN)NC(=O)[C@H](C(C)C)NC(=O)[C@H](CC2CCCCC2)NC(=O)[C@H](CC(N)=O)NC1=O]

Compound 3215 was prepared on a 50 μmol scale. The yield of the product was 7.5 mg, and its estimated purity by LCMS analysis was 95.1%. Analysis condition B: Retention time=1.61 min; ESI-MS(+) m/z [M+3H]³⁺: 799.8.

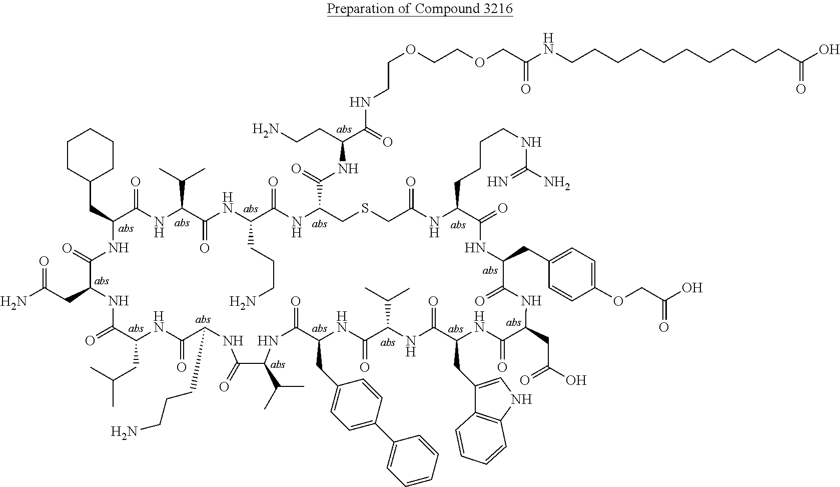 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC1=O]

Compound 3216 was prepared on a 50 μmol scale. The yield of the product was 10.2 mg, and its estimated purity by LCMS analysis was 95.4%. Analysis condition A: Retention time=1.62 min; ESI-MS(+) m/z [M+3H]³⁺: 805.4.

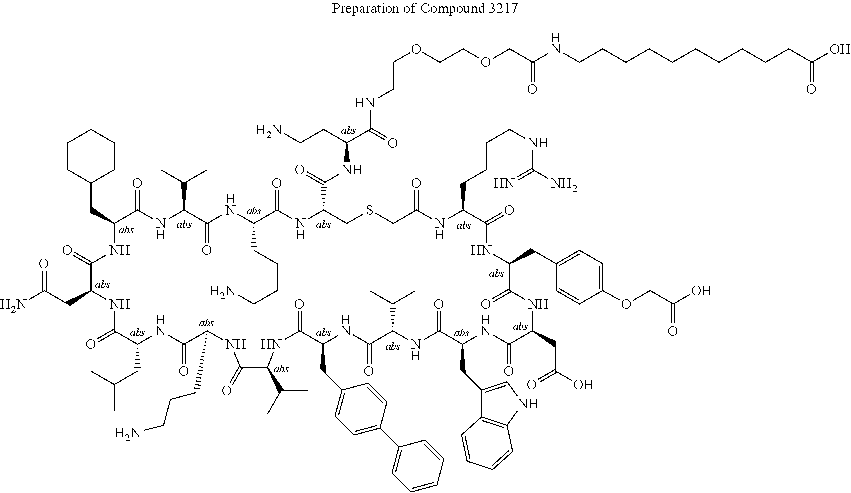 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC2CCCCC2)NC(=O)[C@H](CC(N)=O)NC1=O]

Compound 3217 was prepared on a 50 μmol scale. The yield of the product was 24.9 mg, and its estimated purity by LCMS analysis was 97%. Analysis condition B: Retention time=1.49 min; ESI-MS(+) m/z [M+3H]³⁺: 809.4.

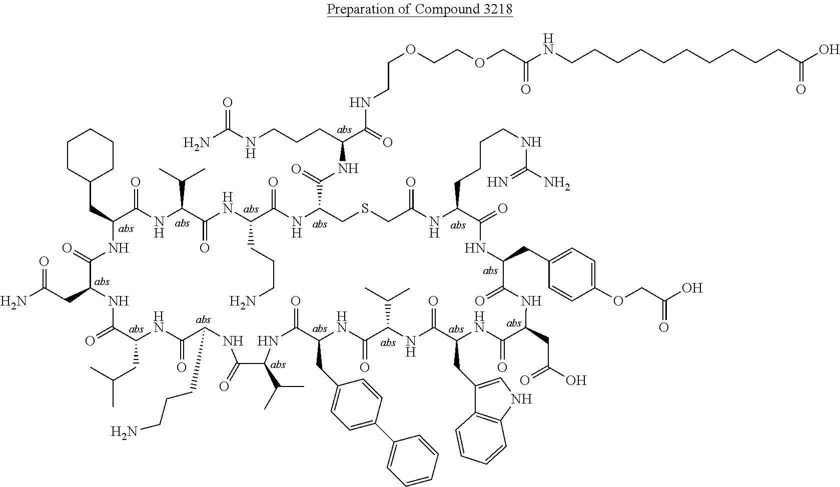 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCCNC(N)=O)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CC(N)=O)NC1=O]

Compound 3218 was prepared on a 50 μmol scale. The yield of the product was 23.5 mg, and its estimated purity by LCMS analysis was 95.4%. Analysis condition A: Retention time=1.59 min; ESI-MS(+) m/z [M+2H]²⁺: 1235.1.

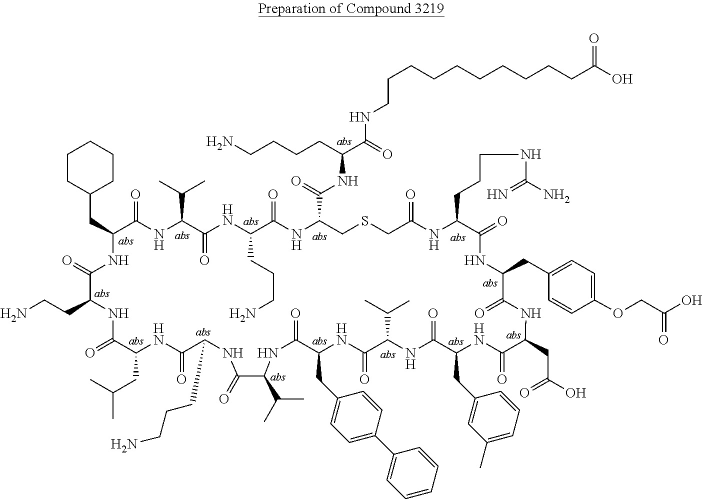 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3219 was prepared on a 50 μmol scale. The yield of the product was 13.4 mg, and its estimated purity by LCMS analysis was 100%. Analysis condition A: Retention time=1.5 min; ESI-MS(+) m/z [M+2H]²⁺: 1122.

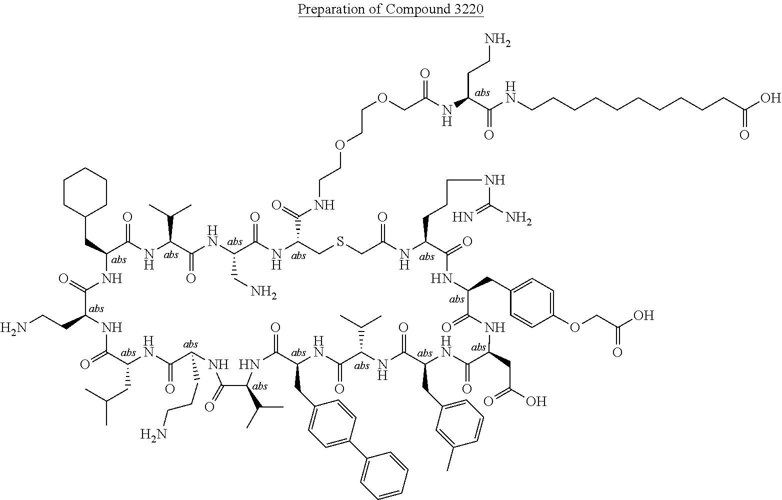 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)N[C@@H](CCN)C(=O)NCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3220 was prepared on a 50 μmol scale. The yield of the product was 7.2 mg, and its estimated purity by LCMS analysis was 96.9%. Analysis condition B: Retention time=1.45 min; ESI-MS(+) m/z [M+3H]³⁺: 777.4.

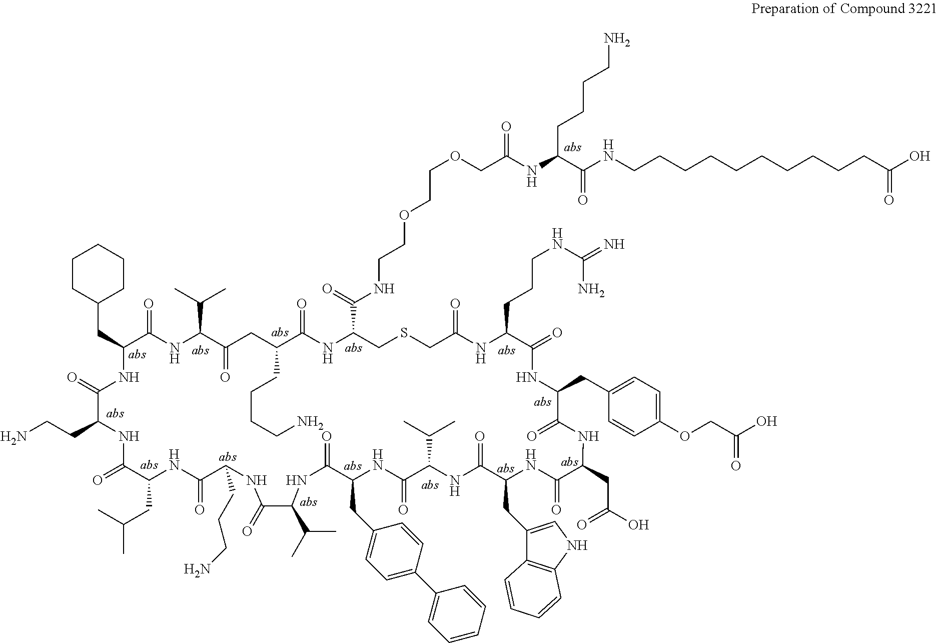 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCOCCOCC(=O)N[C@@H](CCCCN)C(=O)NCCCCCCCCCCC(=O)O)NC(=O)[C@H](CCCCN)CC(=O)[C@H](C(C)C)NC(=O)[C@H](CC2CCCCC2)NC(=O)[C@H](CCN)NC1=O]

Compound 3221 was prepared on a 50 μmol scale. The yield of the product was 11.5 mg, and its estimated purity by LCMS analysis was 100%. Analysis condition A: Retention time=1.38 min; ESI-MS(+) m/z [M+3H]³⁺: 800.2.

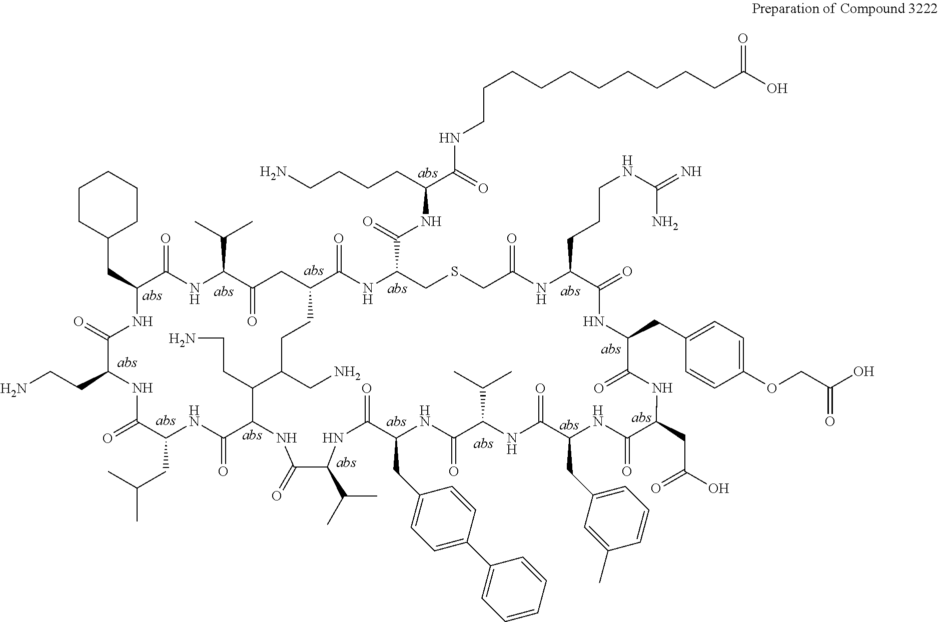 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@@H]3CCC(CN)C(CCN)C(NC(=O)[C@H](C(C)C)NC(=O)[C@H](Cc4ccc(-c5ccccc5)cc4)NC(=O)[C@H](C(C)C)NC2=O)C(=O)N[C@H](CC(C)C)C(=O)N[C@@H](CCN)C(=O)N[C@@H](CC2CCCCC2)C(=O)N[C@@H](C(C)C)C(=O)C3)c1]

Compound 3222 was prepared on a 50 μmol scale. The yield of the product was 4.8 mg, and its estimated purity by LCMS analysis was 96.3%. Analysis condition B: Retention time=1.51 min; ESI-MS(+) m/z [M+2H]²⁺: 1107.3.

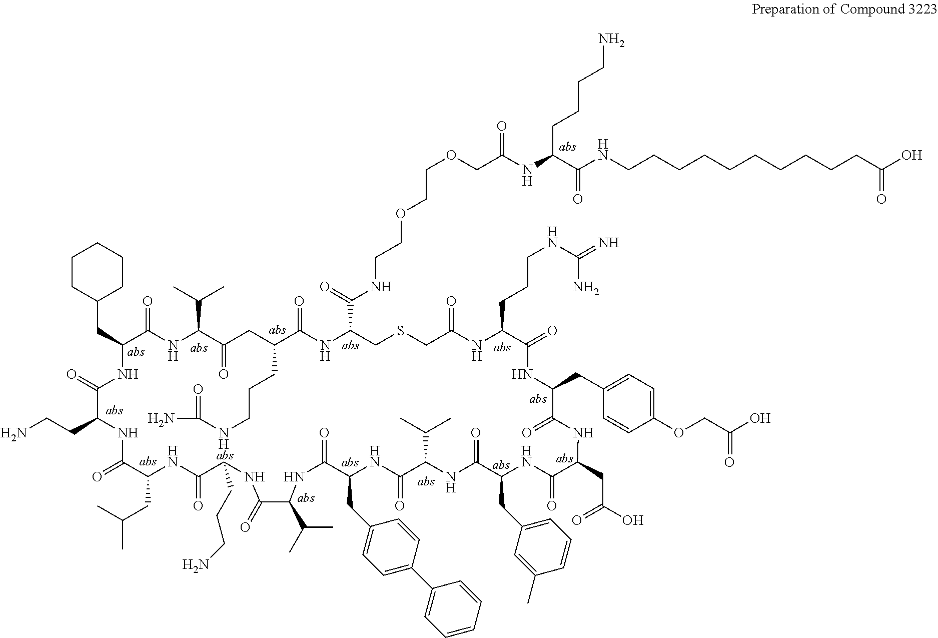 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)N[C@@H](CCCCN)C(=O)NCCCCCCCCCCC(=O)O)NC(=O)[C@H](CCCNC(N)=O)CC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3223 was prepared on a 50 μmol scale. The yield of the product was 7.4 mg, and its estimated purity by LCMS analysis was 100%. Analysis condition A: Retention time=1.7 min; ESI-MS(+) m/z [M+3H]³⁺: 801.4.

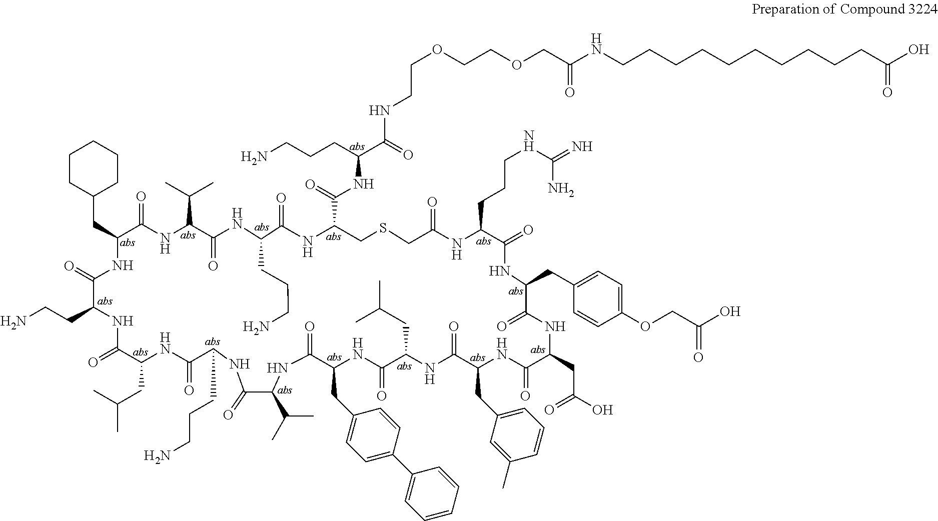

Compound 3224 was prepared on a 50 μmol scale. The yield of the product was 14 mg, and its estimated purity by LCMS analysis was 98.1%. Analysis condition B: Retention time=1.55 min; ESI-MS(+) m/z [M+2H]²⁺: 1187.2.

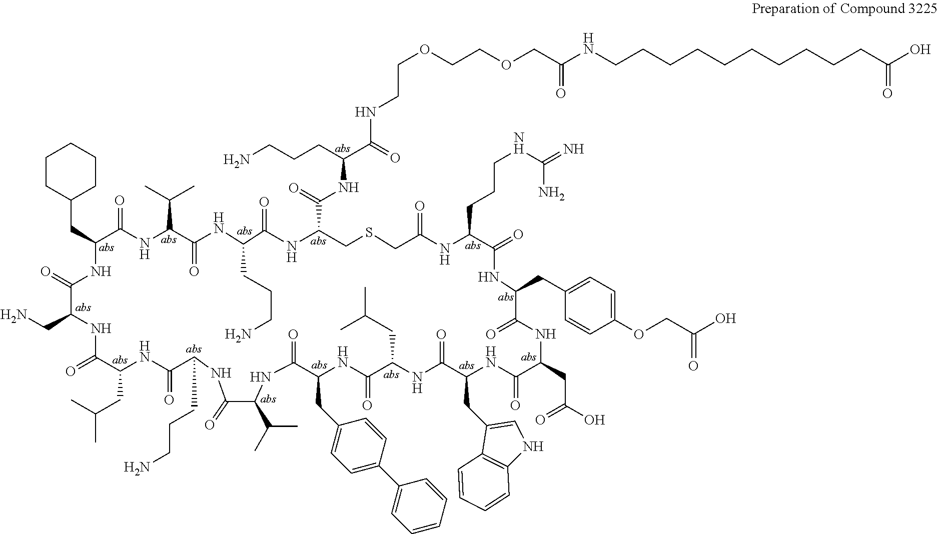

Compound 3225 was prepared on a 50 μmol scale. The yield of the product was 26.9 mg, and its estimated purity by LCMS analysis was 99.4%. Analysis condition B: Retention time=1.38 min; ESI-MS(+) m/z [M+3H]³⁺: 795.2.

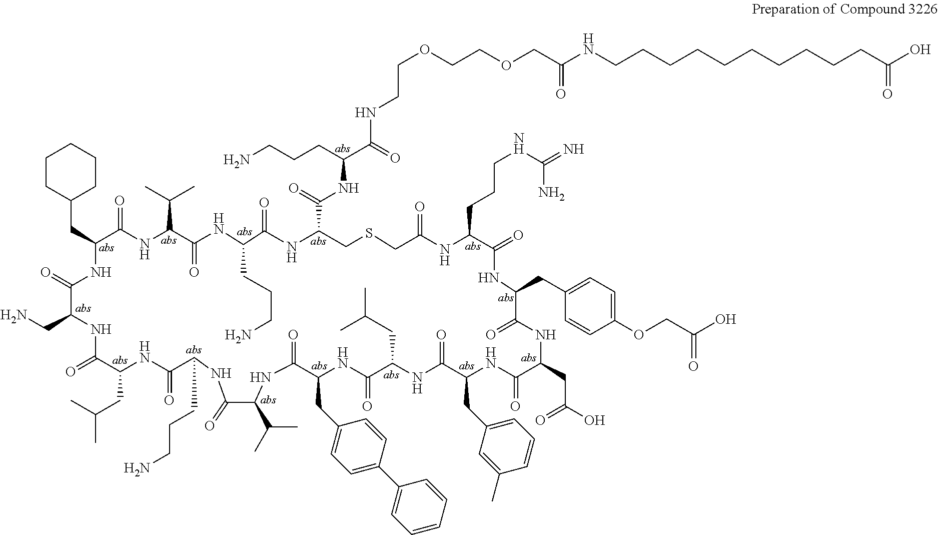

Compound 3226 was prepared on a 50 μmol scale. The yield of the product was 19.4 mg, and its estimated purity by LCMS analysis was 100%. Analysis condition A: Retention time=1.75 min; ESI-MS(+) m/z [M+3H]³⁺: 787.

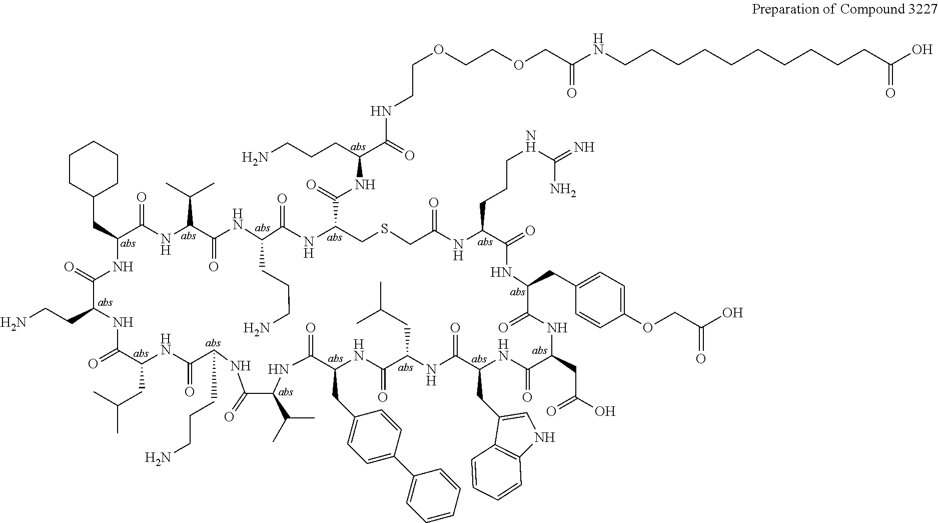

Compound 3227 was prepared on a 50 μmol scale. The yield of the product was 12.2 mg, and its estimated purity by LCMS analysis was 96.9%. Analysis condition B: Retention time=1.4 min; ESI-MS(+) m/z [M+3H]³⁺: 799.5.

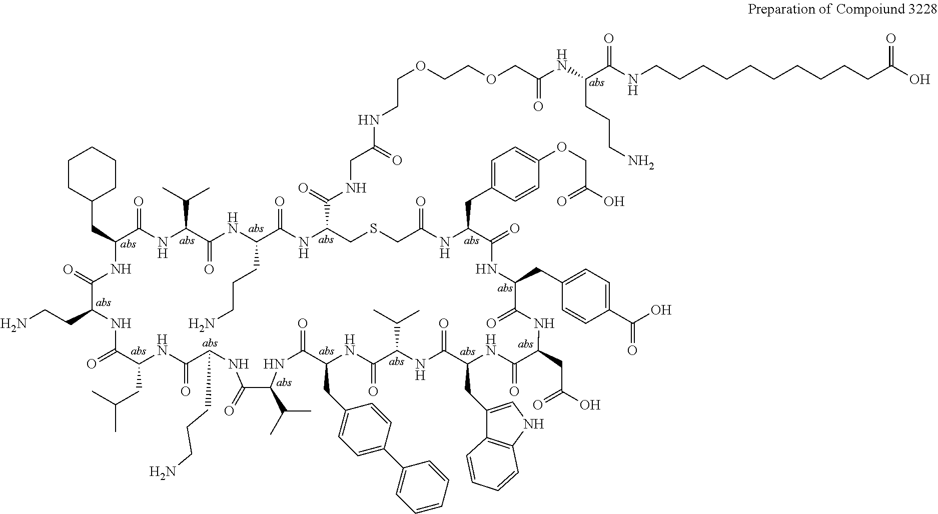 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 3228 was prepared on a 50 μmol scale. The yield of the product was 15.6 mg, and its estimated purity by LCMS analysis was 100%. Analysis condition A: Retention time=1.48 min; ESI-MS(+) m/z [M+2H]²⁺: 1245.9.

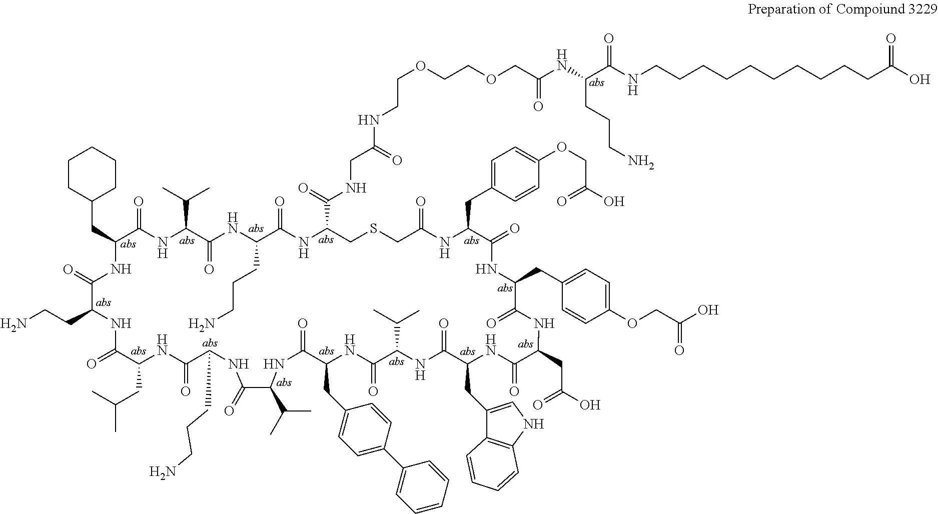 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 3229 was prepared on a 50 μmol scale. The yield of the product was 33.4 mg, and its estimated purity by LCMS analysis was 95.2%. Analysis condition B: Retention time=1.58 min; ESI-MS(+) m/z [M+2H]²⁺: 1261.2.

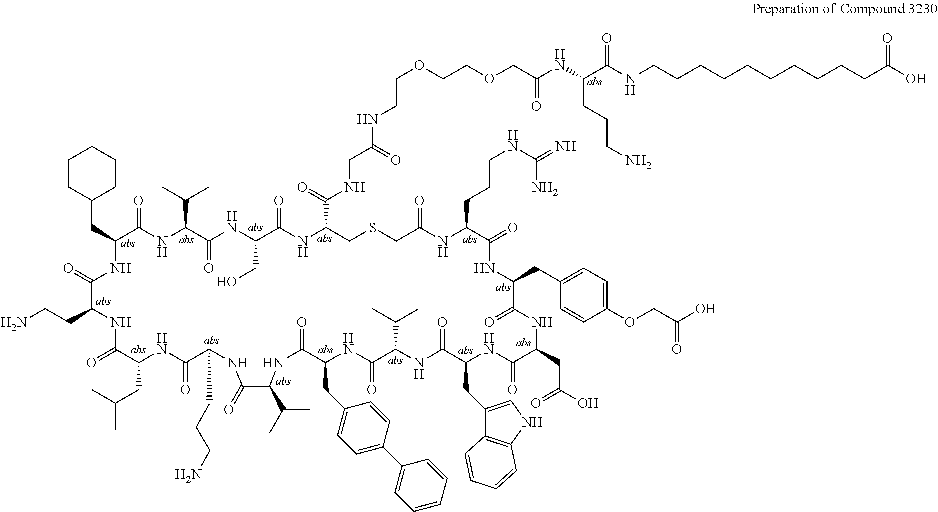 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3230 was prepared on a 50 μmol scale. The yield of the product was 17.9 mg, and its estimated purity by LCMS analysis was 96.3%. Analysis condition B: Retention time=1.42 min; ESI-MS(+) m/z [M+3H]³⁺: 810.2.

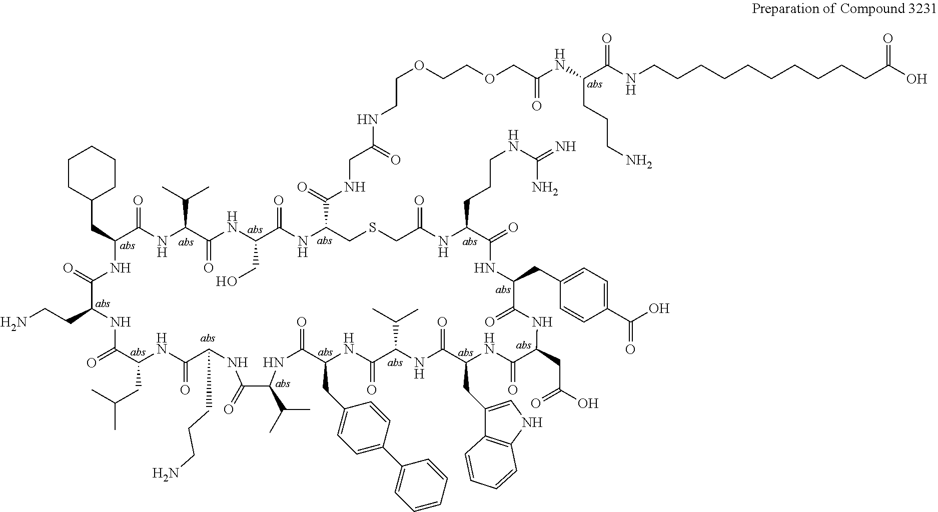 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3231 was prepared on a 50 μmol scale. The yield of the product was 15.1 mg, and its estimated purity by LCMS analysis was 95.5%. Analysis condition A: Retention time=1.56 min; ESI-MS(+) m/z [M+3H]³⁺: 800.3.

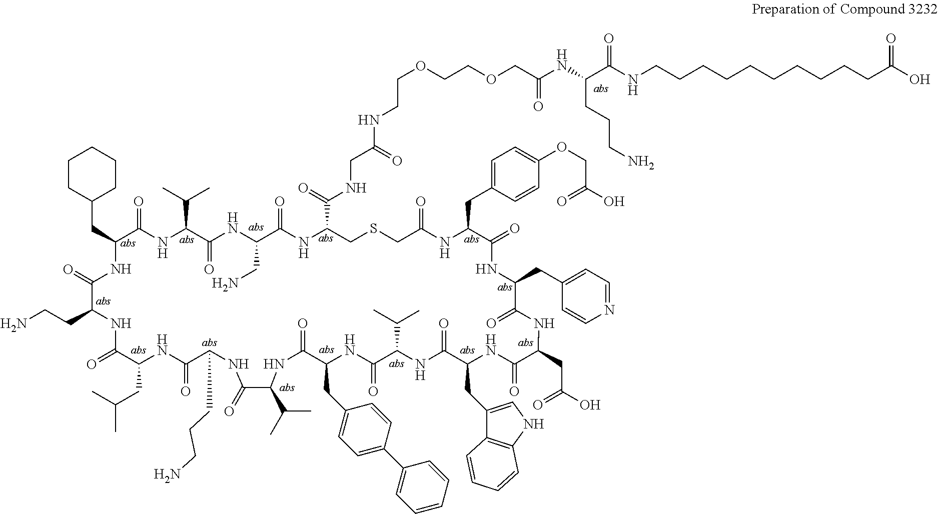 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3232 was prepared on a 50 μmol scale. The yield of the product was 13.2 mg, and its estimated purity by LCMS analysis was 94%. Analysis condition B: Retention time=1.45 min; ESI-MS(+) m/z [M+2H]²⁺: 1210.

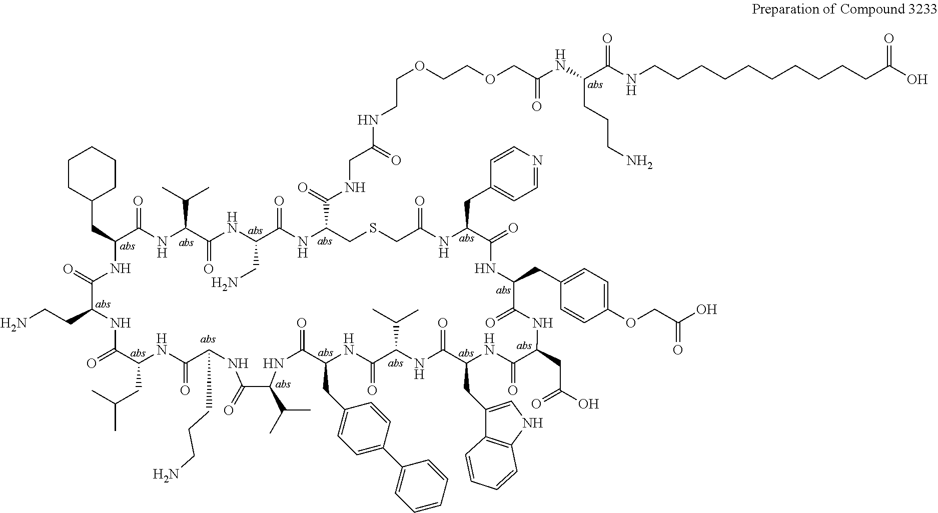 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3233 was prepared on a 50 μmol scale. The yield of the product was 10.4 mg, and its estimated purity by LCMS analysis was 100%. Analysis condition A: Retention time=1.59 min; ESI-MS(+) m/z [M+3H]³⁺: 807.1.

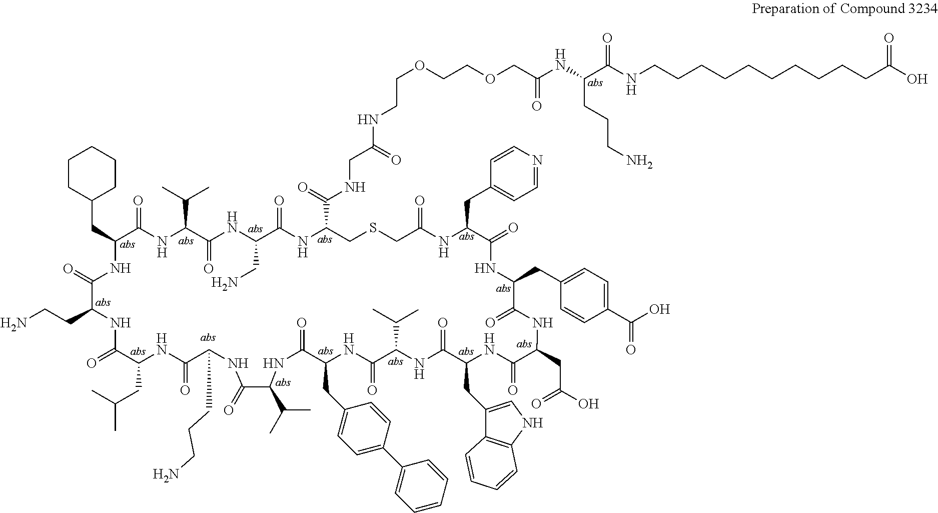 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3234 was prepared on a 50 μmol scale. The yield of the product was 21.4 mg, and its estimated purity by LCMS analysis was 95.2%. Analysis condition B: Retention time=1.33, 1.35 min; ESI-MS(+) m/z [M+3H]³⁺: 797.

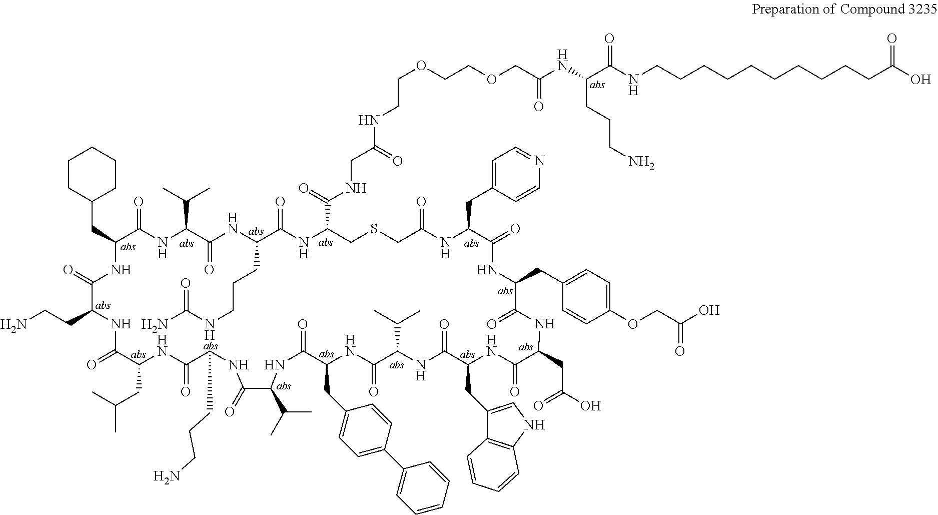 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC1=O]

Compound 3235 was prepared on a 50 μmol scale. The yield of the product was 11.4 mg, and its estimated purity by LCMS analysis was 100%. Analysis condition A: Retention time=1.54 min; ESI-MS(+) m/z [M+3H]³⁺: 831.

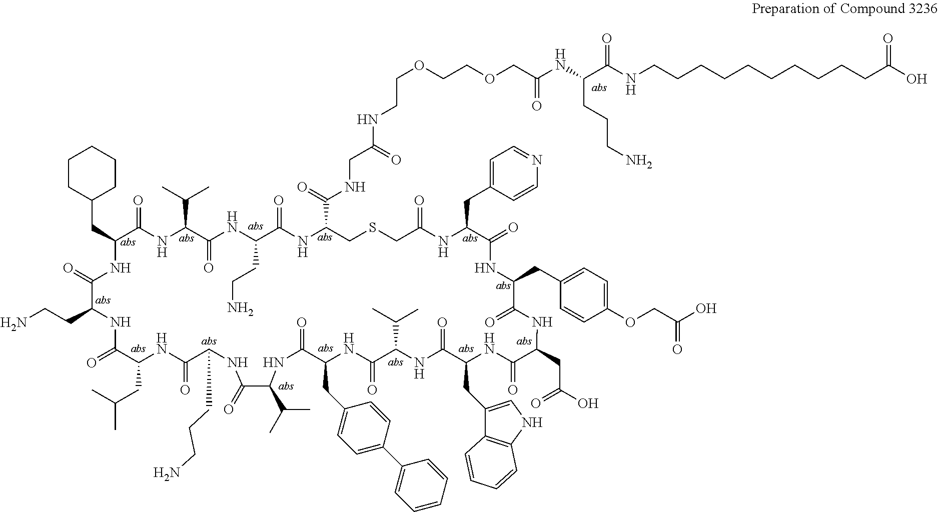 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCN)NC(=O)[C@H](C(C)C)NC(=O)[C@H](CC2CCCCC2)NC(=O)[C@H](CCN)NC1=O]

Compound 3236 was prepared on a 50 μmol scale. The yield of the product was 13.4 mg, and its estimated purity by LCMS analysis was 97%. Analysis condition B: Retention time=1.44 min; ESI-MS(+) m/z [M+3H]³⁺: 812.

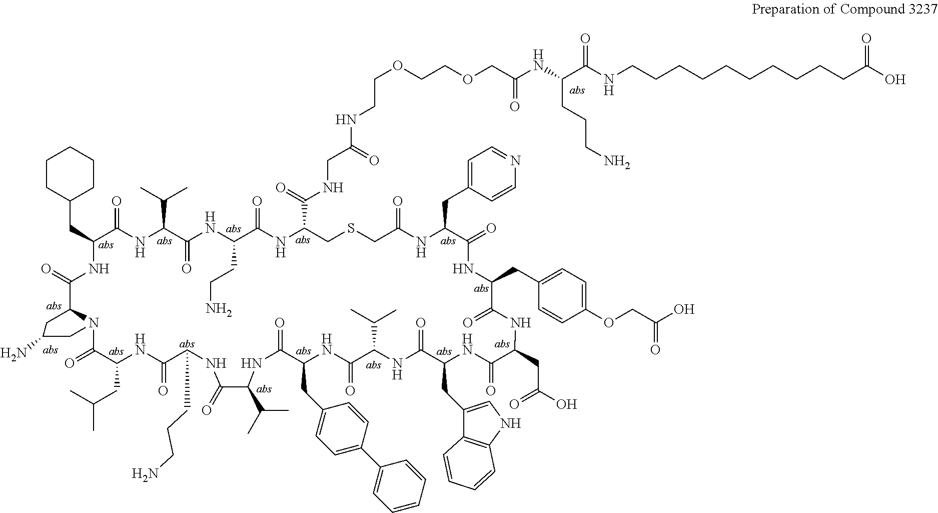 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCC(=O)NCCOCCOCC(=O)N[C@@H](CCCN)C(=O)NCCCCCCCCCCC(=O)O)NC(=O)[C@H](CCN)NC(=O)[C@H](C(C)C)NC(=O)[C@H](CC2CCCCC2)NC(=O)[C@@H]2C[C@@H](N)CN2C1=O]

Compound 3237 was prepared on a 50 μmol scale. The yield of the product was 29 mg, and its estimated purity by LCMS analysis was 97.6%. Analysis condition A: Retention time=1.55 min; ESI-MS(+) m/z [M+2H]²⁺: 1223.1.

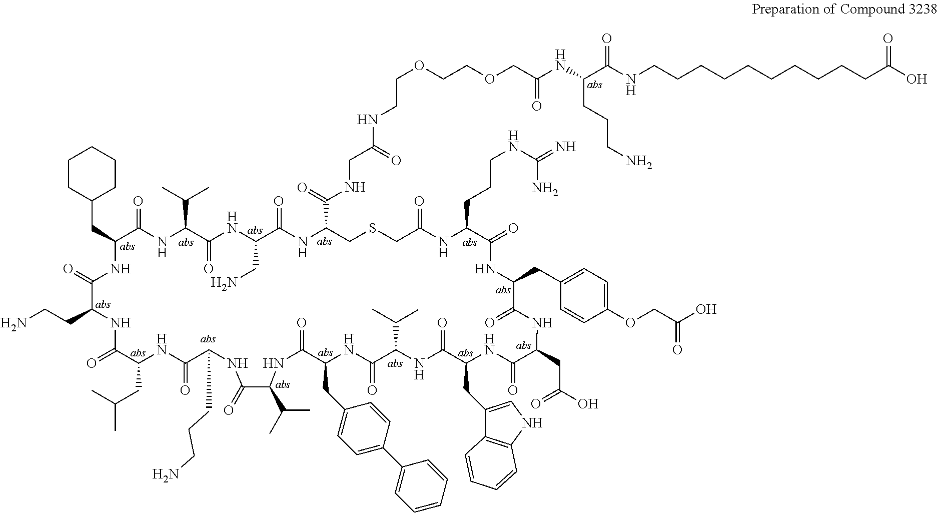 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3238 was prepared on a 50 μmol scale. The yield of the product was 12.6 mg, and its estimated purity by LCMS analysis was 100%. Analysis condition A: Retention time=1.51 min; ESI-MS(+) m/z [M+2H]²⁺: 1213.8.

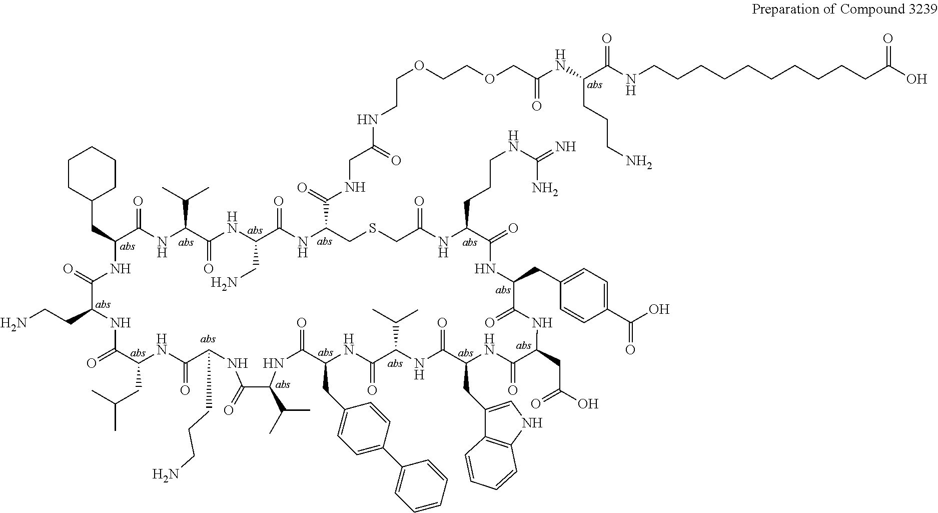 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3239 was prepared on a 50 μmol scale. The yield of the product was 12.6 mg, and its estimated purity by LCMS analysis was 100%. Analysis condition A: Retention time=1.5 min; ESI-MS(+) m/z [M+2H]²⁺: 1199.1.

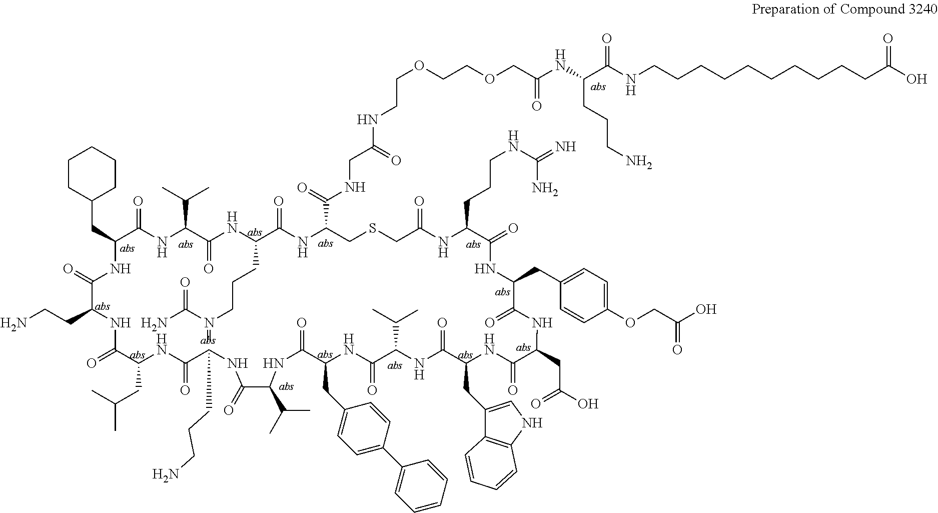

Compound 3240 was prepared on a 50 μmol scale. The yield of the product was 2.3 mg, and its estimated purity by LCMS analysis was 96.2%. Analysis condition A: Retention time=1.54 min; ESI-MS(+) m/z [M+3H]³⁺: 833.2.

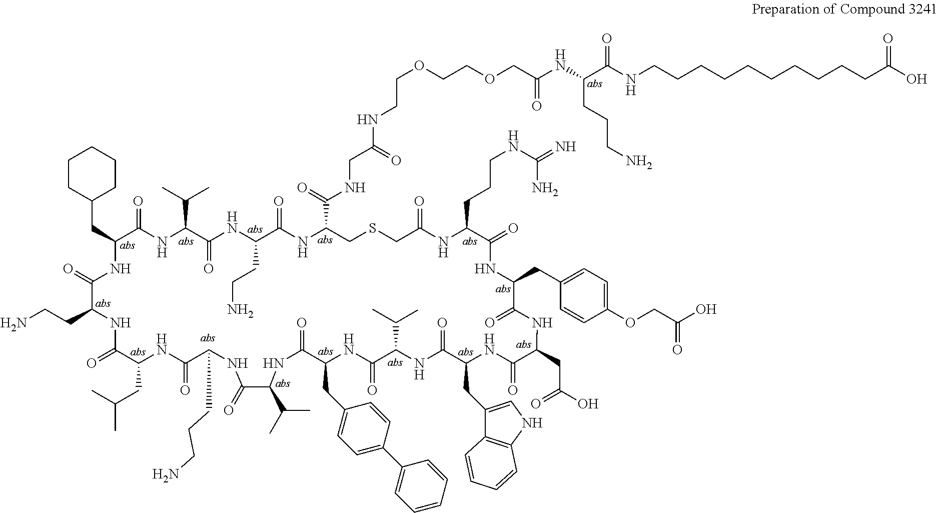 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCN)NC(=O)[C@H](C(C)C)NC(=O)[C@H](CC2CCCCC2)NC(=O)[C@H](CCN)NC1=O]

Compound 3241 was prepared on a 50 μmol scale. The yield of the product was 13.2 mg, and its estimated purity by LCMS analysis was 100%. Analysis condition A: Retention time=1.47 min; ESI-MS(+) m/z [M+2H]²⁺: 1221.1.

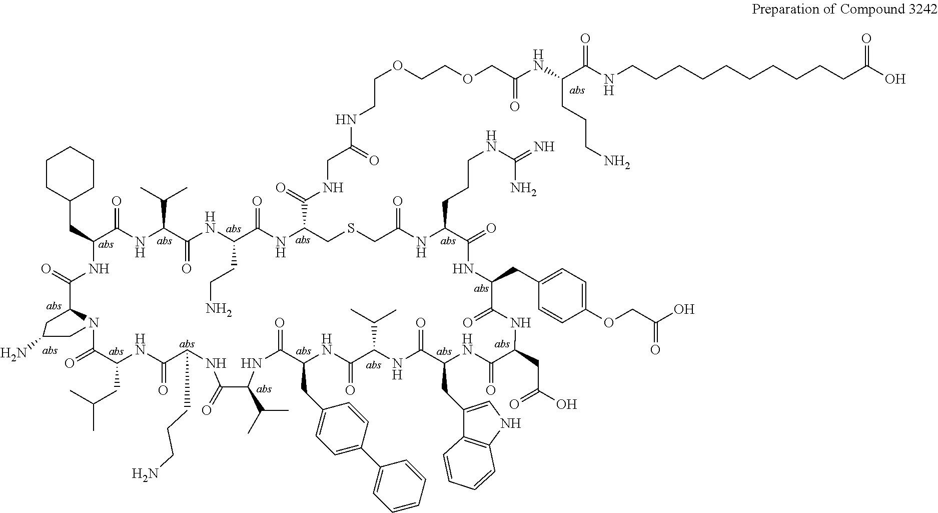 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCC(=O)NCCOCCOCC(=O)N[C@@H](CCCN)C(=O)NCCCCCCCCCCC(=O)O)NC(=O)[C@H](CCN)NC(=O)[C@H](C(C)C)NC(=O)[C@H](CC2CCCCC2)NC(=O)[C@@H]2C[C@@H](N)CN2C1=O]

Compound 3242 was prepared on a 50 μmol scale. The yield of the product was 25.8 mg, and its estimated purity by LCMS analysis was 97.5%. Analysis condition A: Retention time=1.51 min; ESI-MS(+) m/z [M+2H]²⁺: 1227.

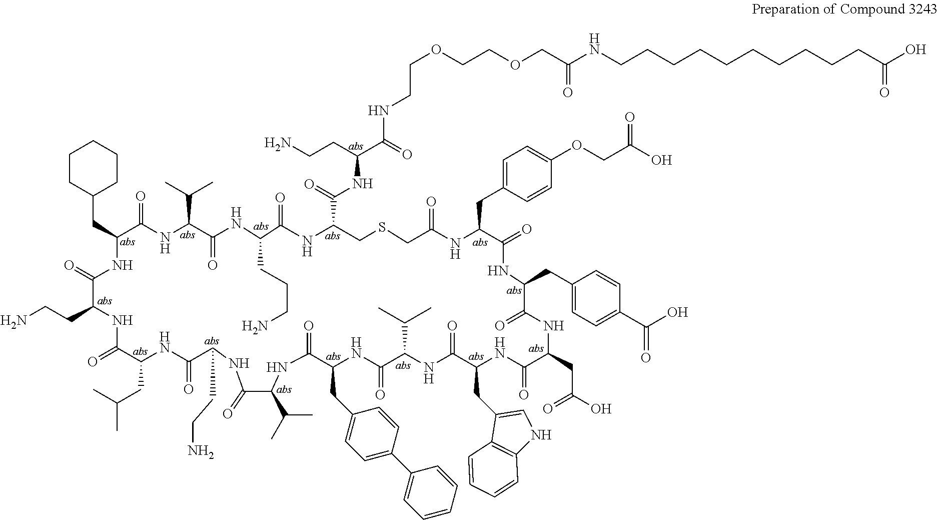 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 3243 was prepared on a 50 μmol scale. The yield of the product was 17.1 mg, and its estimated purity by LCMS analysis was 95.4%. Analysis condition B: Retention time-1.65 min; ESI-MS(+) m/z [M+2H]²⁺: 1203.1.

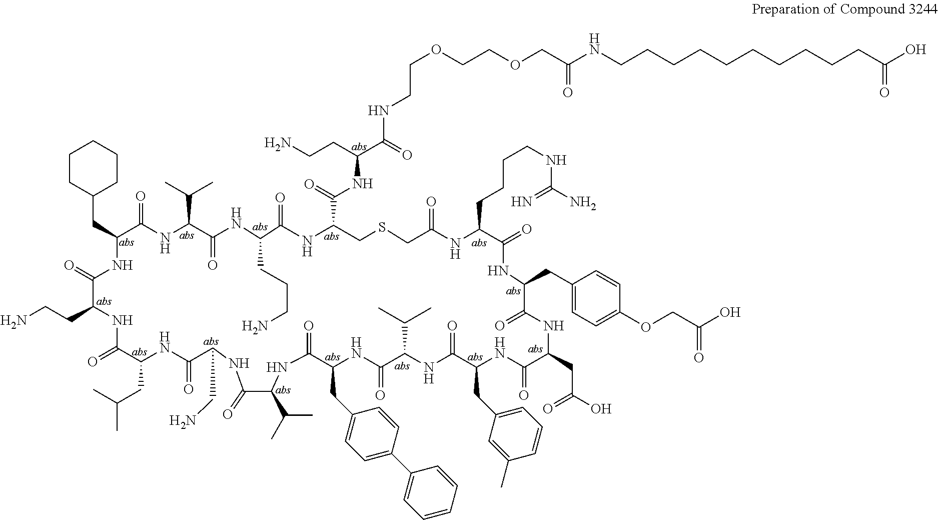 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CN)NC(=O)[C@H](C(C)C)NC(=O)[C@H](Cc3ccc(-c4ccccc4)cc3)NC(=O)[C@H](C(C)C)NC2=O)c1]

Compound 3244 was prepared on a 50 μmol scale. The yield of the product was 16.9 mg, and its estimated purity by LCMS analysis was 95%. Analysis condition B: Retention time=1.56 min; ESI-MS(+) m/z [M+2H]²⁺: 1173.2.

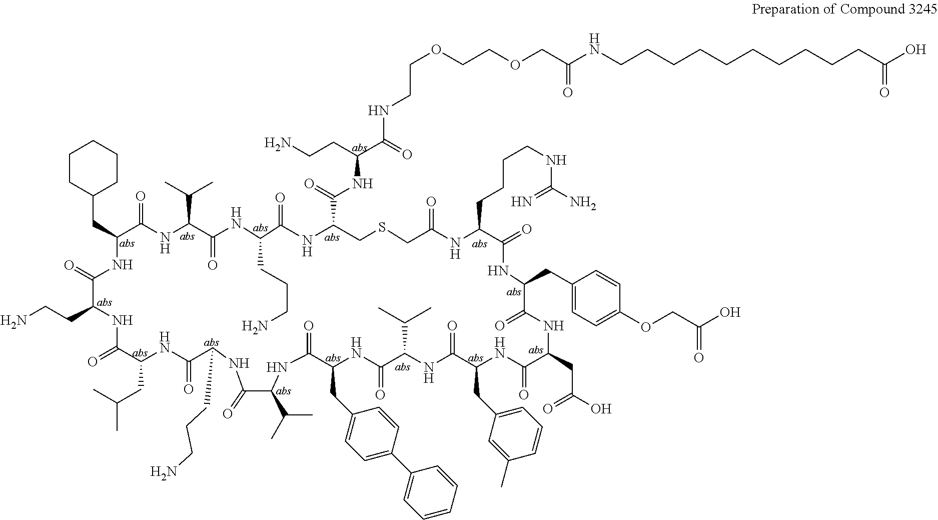 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3245 was prepared on a 50 μmol scale. The yield of the product was 6.1 mg, and its estimated purity by LCMS analysis was 95.2%. Analysis condition A: Retention time=1.5 min; ESI-MS(+) m/z [M+2H]²⁺: 1187.1.

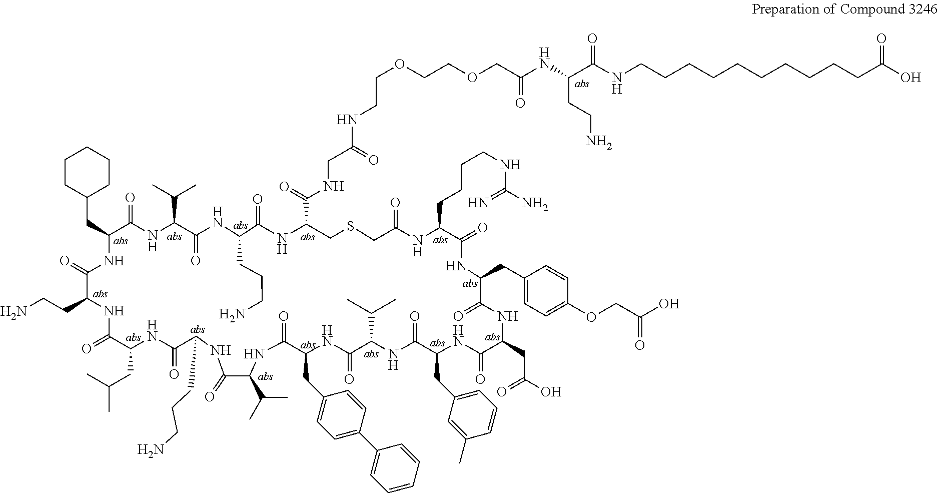 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCC(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3246 was prepared on a 50 μmol scale. The yield of the product was 20.7 mg, and its estimated purity by LCMS analysis was 99.2%. Analysis condition A: Retention time=1.49 min; ESI-MS(+) m/z [M+2H]²⁺: 1216.

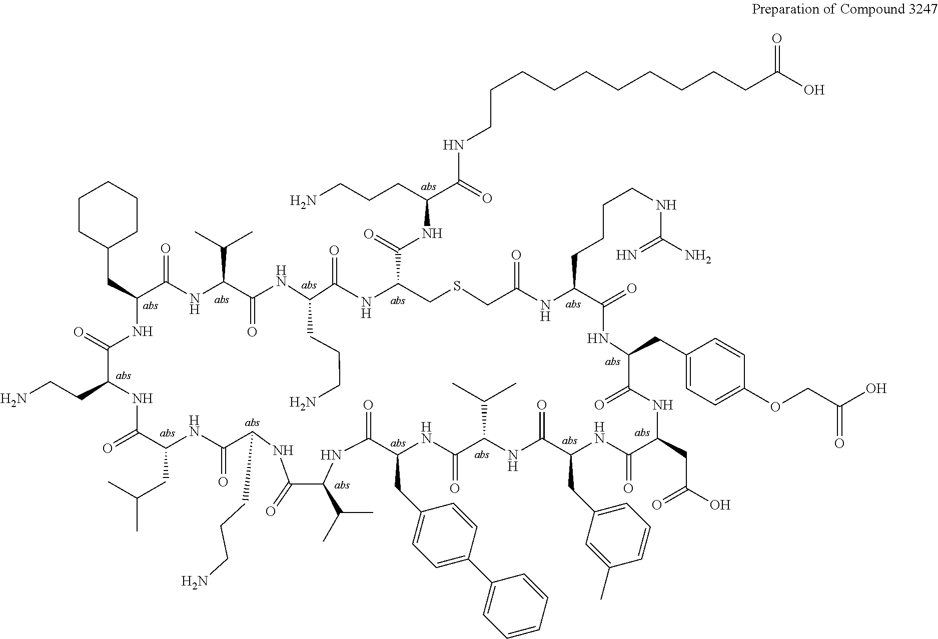 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3247 was prepared on a 50 μmol scale. The yield of the product was 11 mg, and its estimated purity by LCMS analysis was 97.7%. Analysis condition B: Retention time=1.43 min; ESI-MS(+) m/z [M+2H]²⁺: 1121.5.

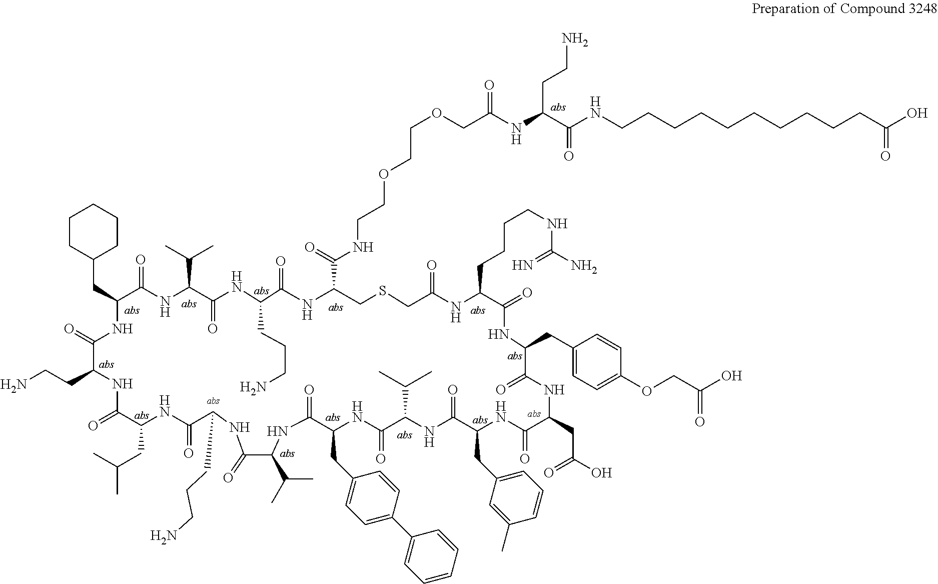 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3248 was prepared on a 50 μmol scale. The yield of the product was 13.4 mg, and its estimated purity by LCMS analysis was 100%. Analysis condition A: Retention time=1.51 min; ESI-MS(+) m/z [M+2H]²⁺: 1187.1.

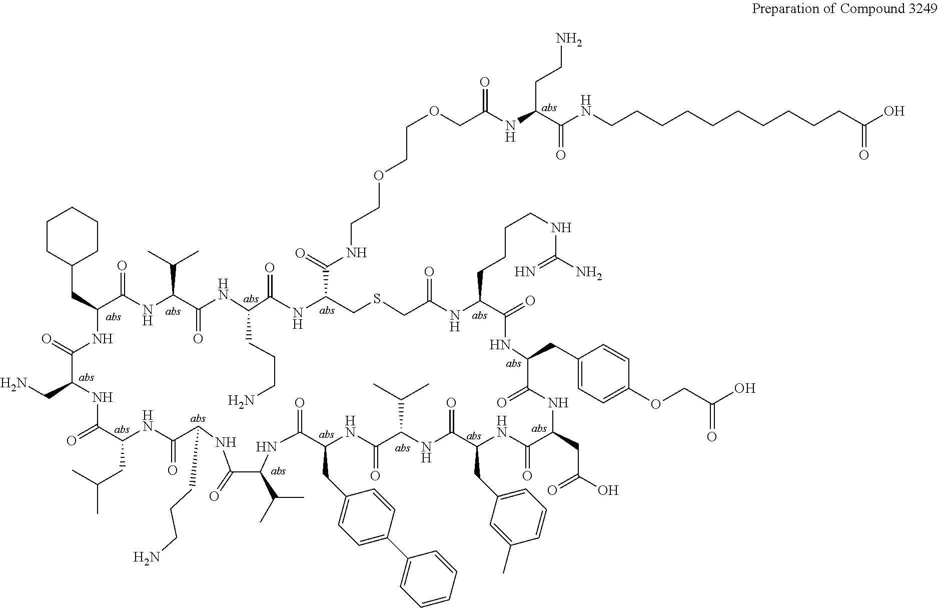 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3249 was prepared on a 50 μmol scale. The yield of the product was 17.2 mg, and its estimated purity by LCMS analysis was 96.1%. Analysis condition A: Retention time-1.55 min; ESI-MS(+) m/z [M+2H]²⁺: 1180.

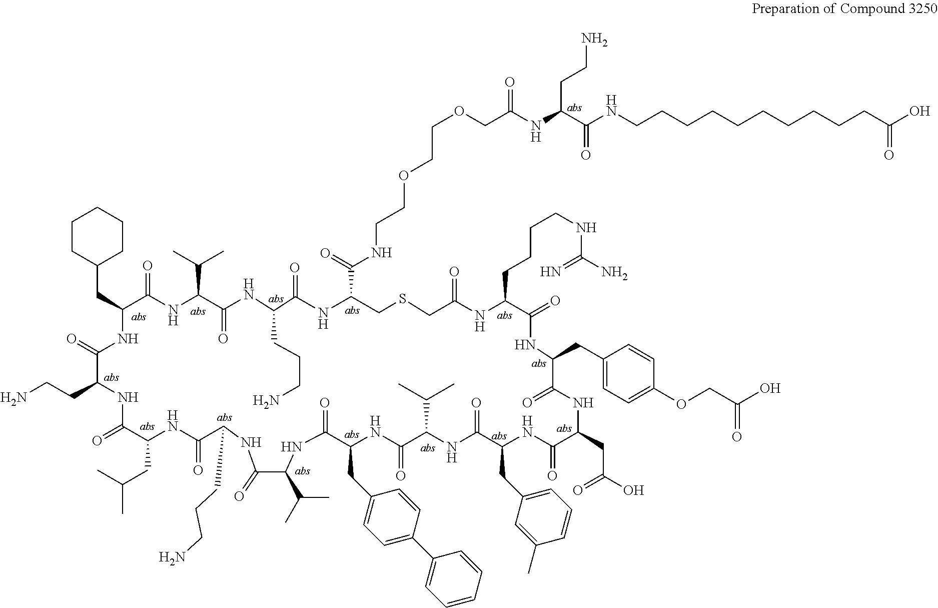 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3250 was prepared on a 50 μmol scale. The yield of the product was 6.1 mg, and its estimated purity by LCMS analysis was 96.1%. Analysis condition B: Retention time=1.39 min; ESI-MS(+) m/z [M+2H]²⁺: 1173.1.

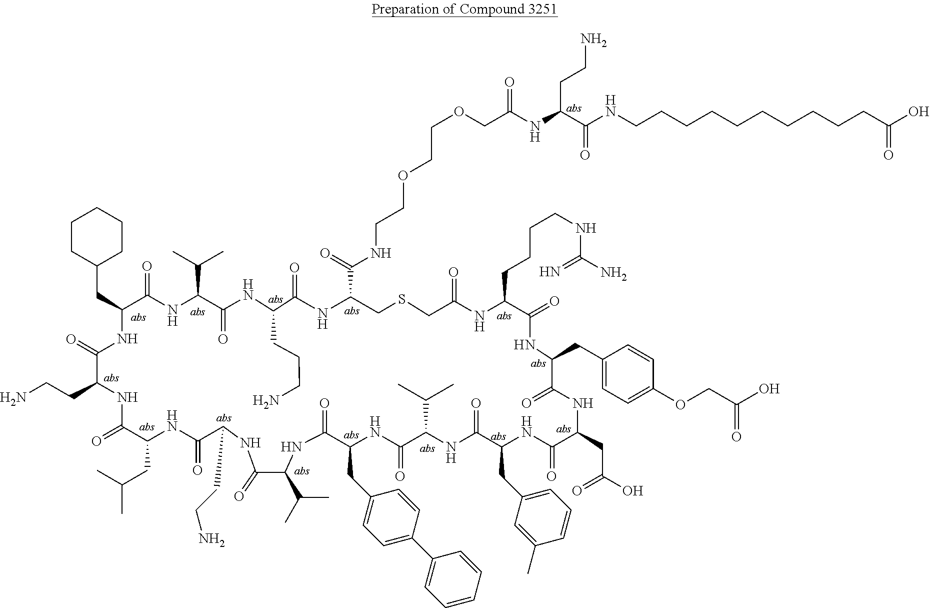 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3251 was prepared on a 50 μmol scale. The yield of the product was 5.9 mg, and its estimated purity by LCMS analysis was 100%. Analysis condition A: Retention time=1.53 min; ESI-MS(+) m/z [M+3H]³⁺: 787.2.

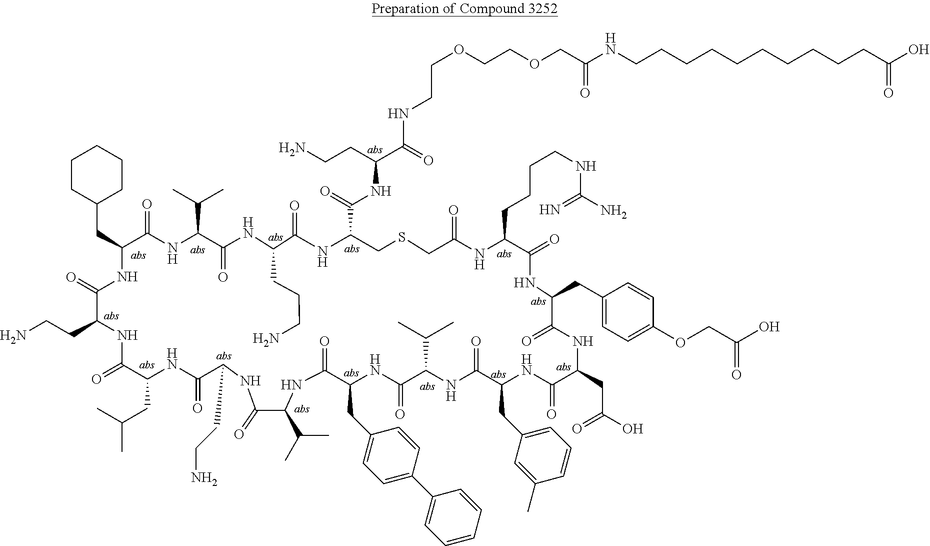 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3252 was prepared on a 50 μmol scale. The yield of the product was 8.8 mg, and its estimated purity by LCMS analysis was 96.3%. Analysis condition B: Retention time=1.53 min; ESI-MS(+) m/z [M+2H]²⁺: 1180.1.

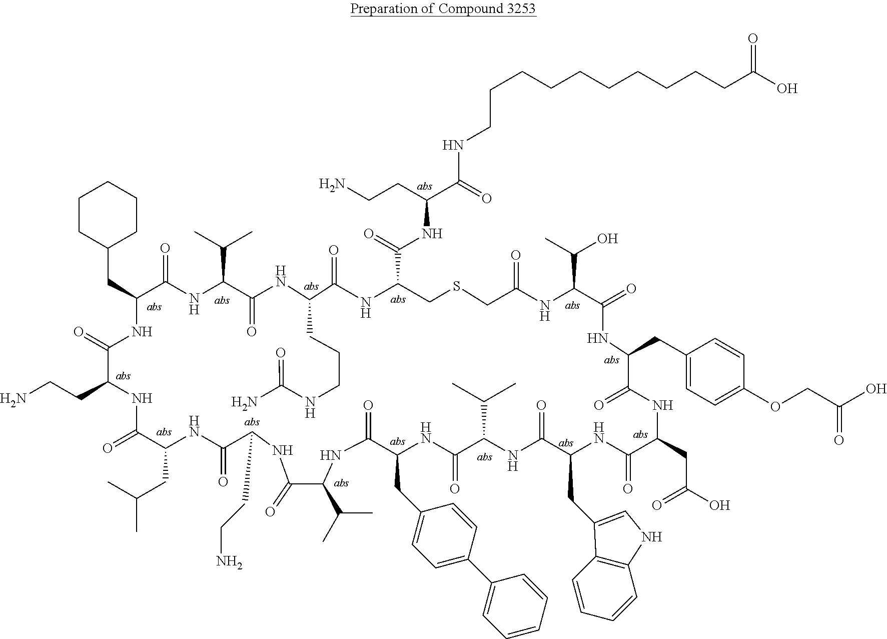 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC1=O]

Compound 3253 was prepared on a 50 μmol scale. The yield of the product was 21.2 mg, and its estimated purity by LCMS analysis was 94.9%. Analysis condition A: Retention time=1.49 min; ESI-MS(+) m/z [M+2H]²⁺: 1107.3.

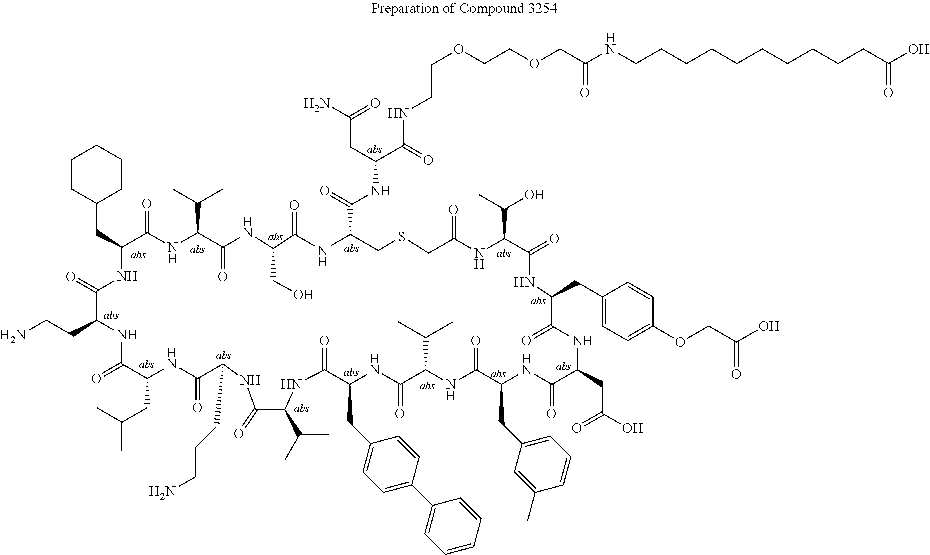 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCOCCOCC(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3254 was prepared on a 50 μmol scale. The yield of the product was 3.3 mg, and its estimated purity by LCMS analysis was 94.7%. Analysis condition A: Retention time=1.74 min; ESI-MS(+) m/z [M+2H]²⁺: 1145.4.

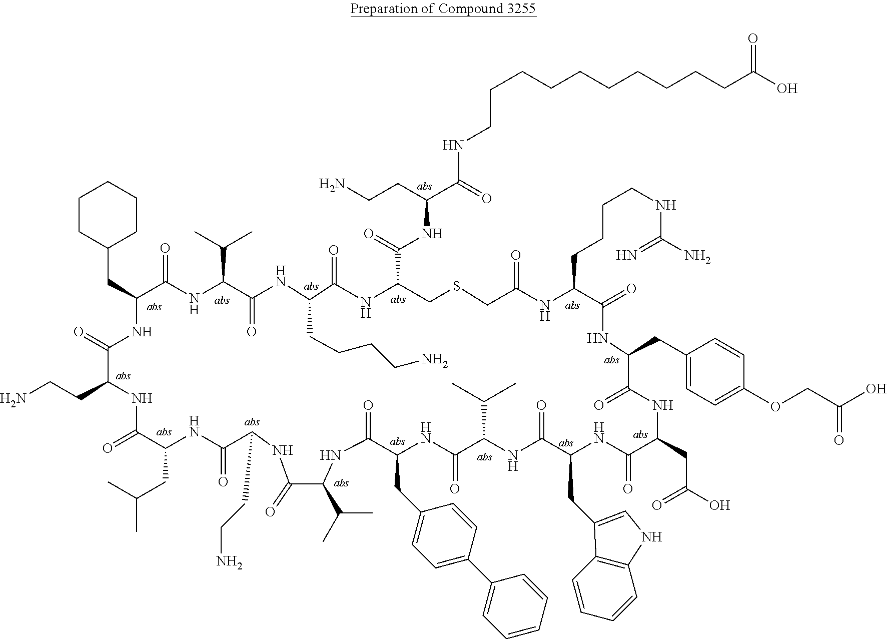 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 3255 was prepared on a 50 μmol scale. The yield of the product was 5.9 mg, and its estimated purity by LCMS analysis was 97.6%. Analysis condition A: Retention time=1.51 min; ESI-MS(+) m/z [M+2H]²⁺: 1127.1.

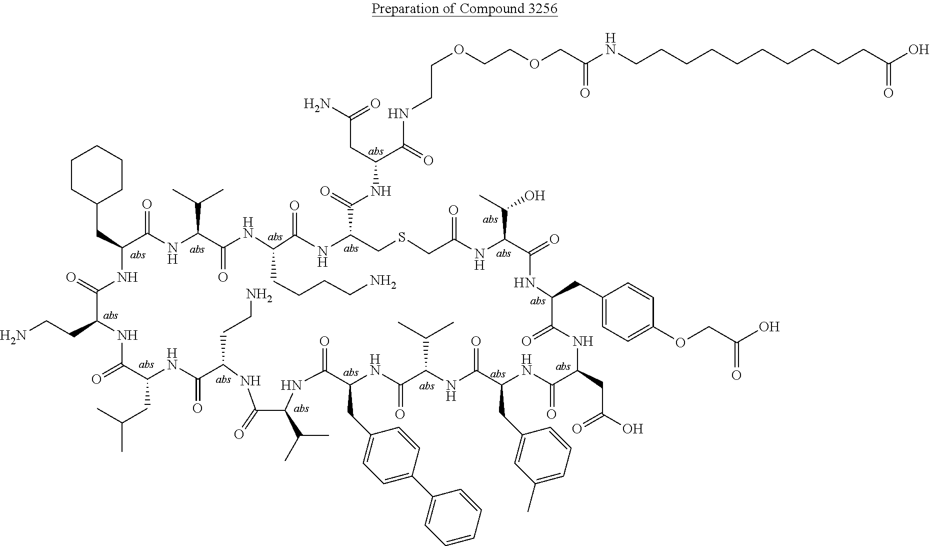 [SMILES: Cc1cccc(C[C@@H]2NC(=O)[C@H](CC(=O)O)NC(=O)[C@H](Cc3ccc(OCC(=O)O)cc3)NC(=O)[C@H]([C@H](C)O)NC(=O)CSC[C@@H](C(=O)N[C@H](CC(N)=O)C(=O)NCCOCCOCC(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3256 was prepared on a 50 μmol scale. The yield of the product was 12.4 mg, and its estimated purity by LCMS analysis was 95.9%. Analysis condition A: Retention time=1.7 min; ESI-MS(+) m/z [M+3H]³⁺: 773.2.

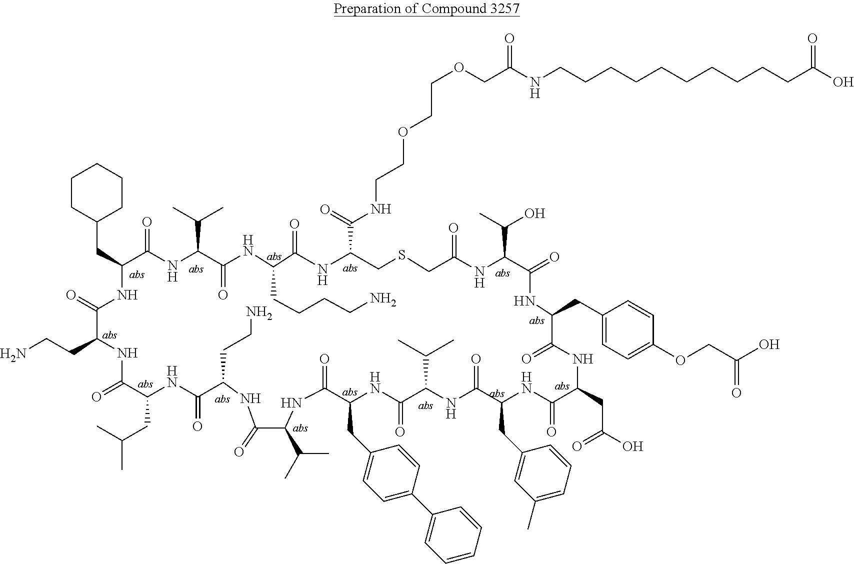 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3257 was prepared on a 50 μmol scale. The yield of the product was 8.5 mg, and its estimated purity by LCMS analysis was 100%. Analysis condition A: Retention time=1.7 min; ESI-MS(+) m/z [M+3H]³⁺: 735.2.

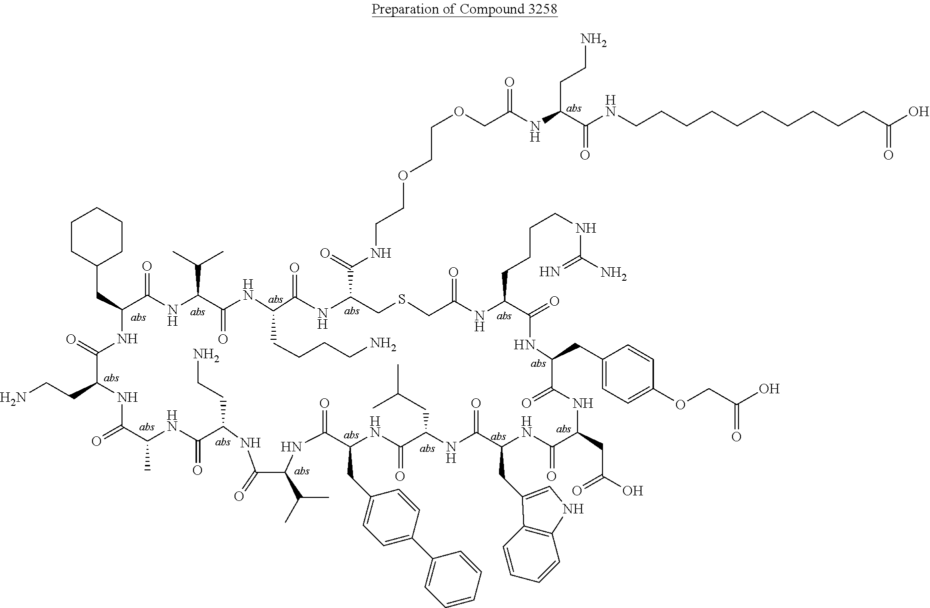 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 3258 was prepared on a 50 μmol scale. The yield of the product was 20.8 mg, and its estimated purity by LCMS analysis was 96.7%. Analysis condition B: Retention time=1.4 min; ESI-MS(+) m/z [M+2H]²⁺: 1185.4.

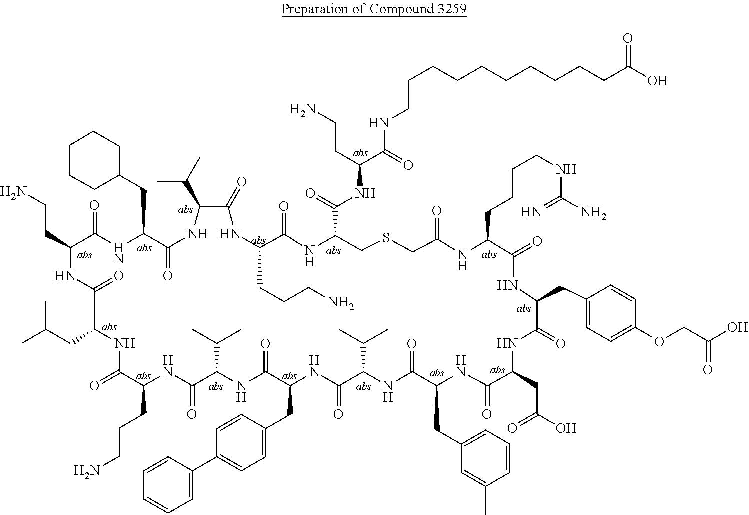 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3259 was prepared on a 50 μmol scale. The yield of the product was 21 mg, and its estimated purity by LCMS analysis was 95.8%. Analysis condition A: Retention time=1.34 min; ESI-MS(+) m/z [M+3H]³⁺: 743.6.

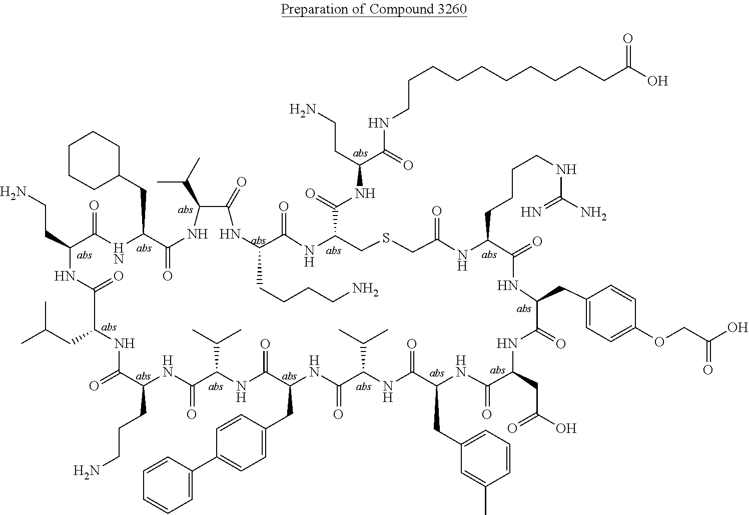 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3260 was prepared on a 50 μmol scale. The yield of the product was 8 mg, and its estimated purity by LCMS analysis was 98.3%. Analysis condition B: Retention time=1.46 min; ESI-MS(+) m/z [M+3H]³⁺: 748.1.

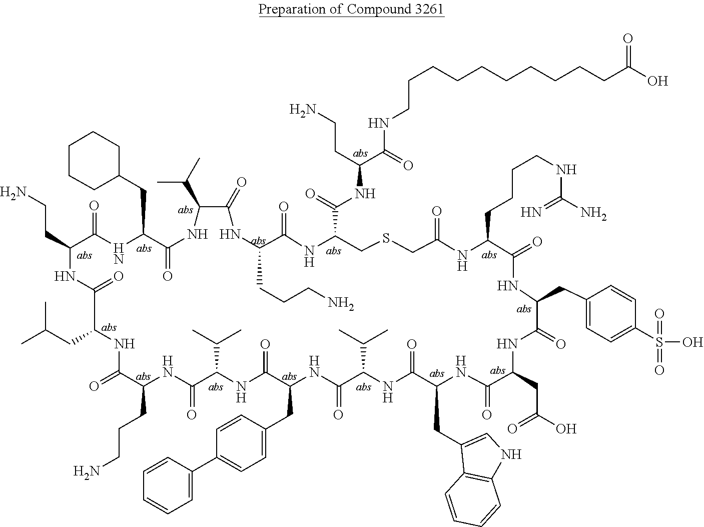 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(S(=O)(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Compound 3261 was prepared on a 50 μmol scale. The yield of the product was 12.3 mg, and its estimated purity by LCMS analysis was 96.4%. Analysis condition B: Retention time=1.45 min; ESI-MS(+) m/z [M+2H]²⁺: 1127.2.

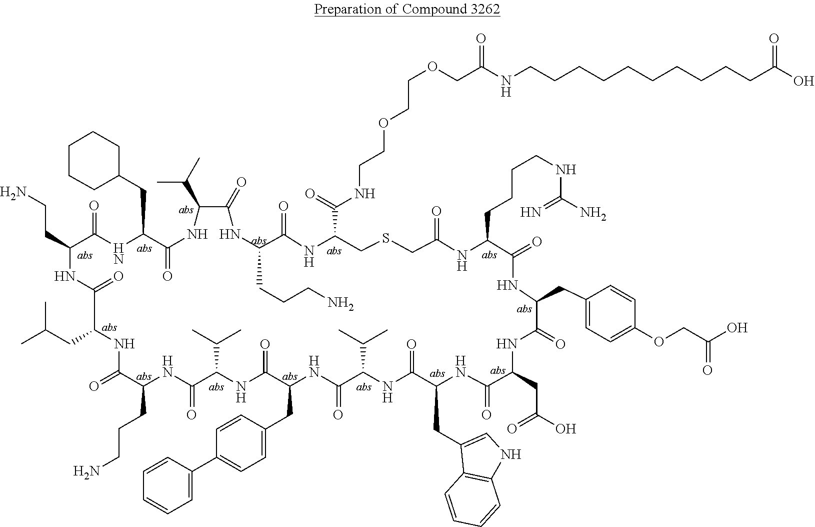 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)NCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Compound 3262 was prepared on a 50 μmol scale. The yield of the product was 19.5 mg, and its estimated purity by LCMS analysis was 100%. Analysis condition A: Retention time=1.54 min; ESI-MS(+) m/z [M+3H]³⁺: 766.9.

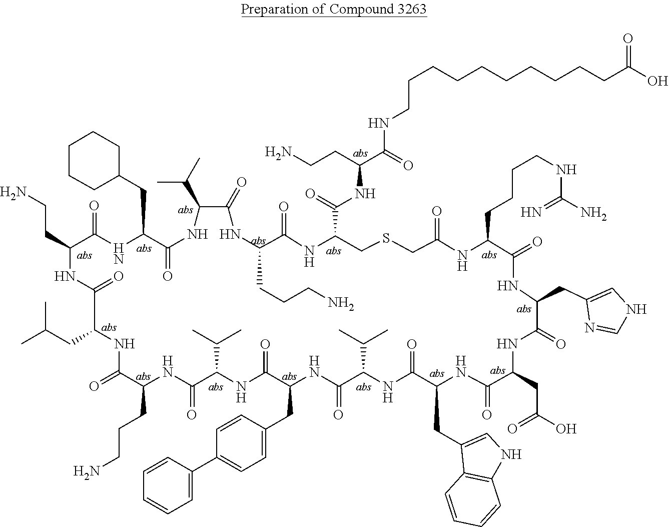 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]cn2)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Compound 3263 was prepared on a 50 μmol scale. The yield of the product was 51.9 mg, and its estimated purity by LCMS analysis was 100%. Analysis condition A: Retention time=1.43 min; ESI-MS(+) m/z [M+2H]²⁺: 1085.1.

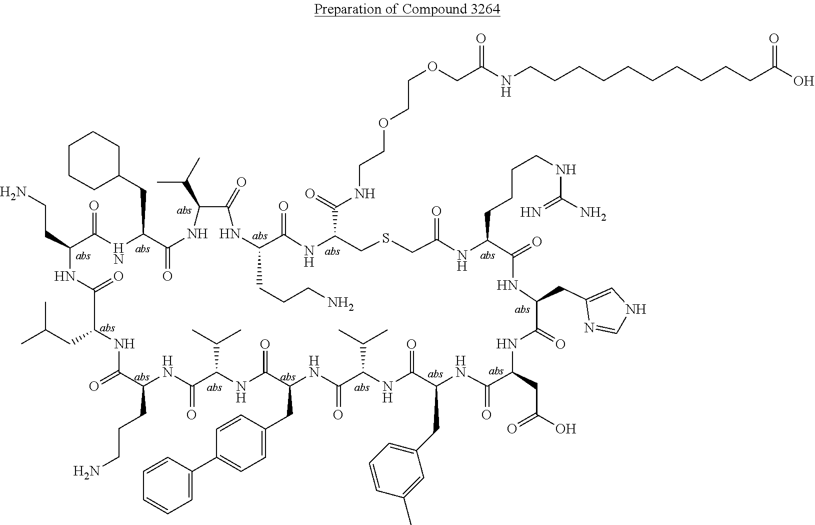 [SMILES: Cc1cccc(C[C@@H]2NC(=O)[C@H](CC(=O)O)NC(=O)[C@H](Cc3c[nH]cn3)NC(=O)[C@H](CCCCNC(=N)N)NC(=O)CSC[C@@H](C(=O)NCCOCCOCC(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3264 was prepared on a 50 μmol scale. The yield of the product was 31 mg, and its estimated purity by LCMS analysis was 98.5%. Analysis condition B: Retention time=1.45 min; ESI-MS(+) m/z [M+2H]²⁺: 1095.1.

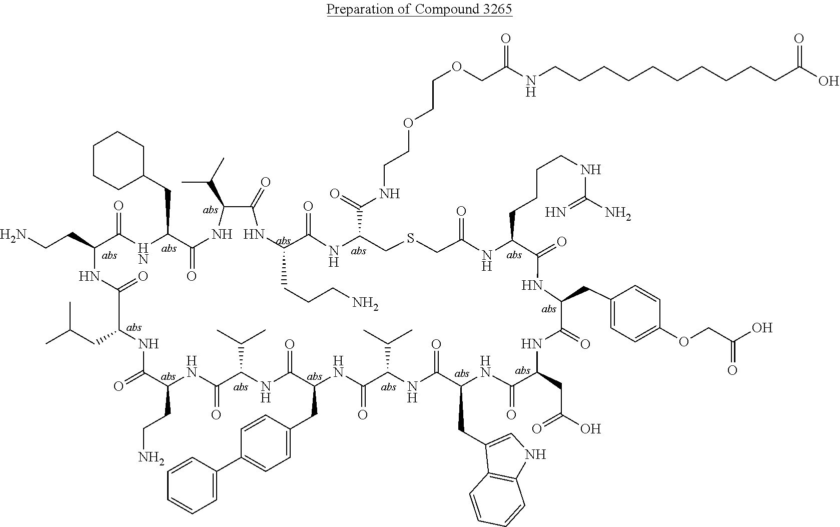 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)NCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Compound 3265 was prepared on a 50 μmol scale. The yield of the product was 15.2 mg, and its estimated purity by LCMS analysis was 95.2%. Analysis condition B: Retention time=1.54 min; ESI-MS(+) m/z [M+3H]³⁺: 762.2.

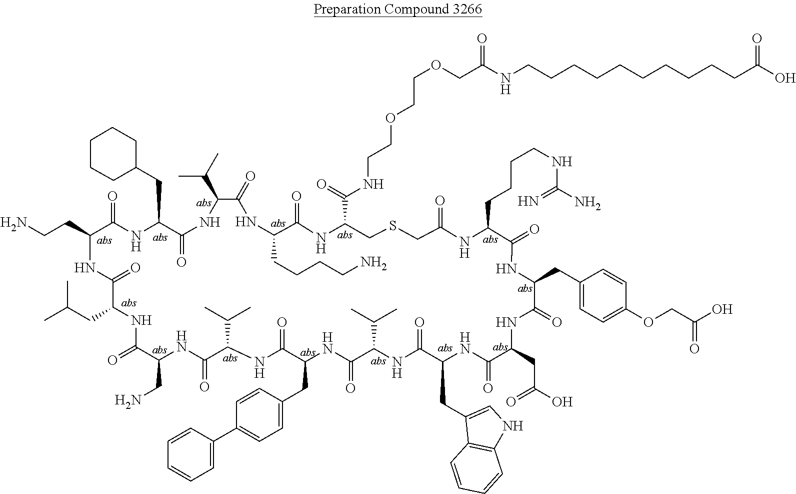 [SMILES: CC(C)C[C@H]1NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)NCCOCCOCC(=O)NCCCCCCCCCCC(=O)O)NC(=O)C[C@H](CCCCN)NC(=O)[C@H](C(C)C)NC(=O)[C@H](CC2CCCCC2)NC(=O)[C@H](CCN)NC1=O]

Compound 3266 was prepared on a 50 μmol scale. The yield of the product was 30.9 mg, and its estimated purity by LCMS analysis was 97.2%. Analysis condition A: Retention time=1.57 min; ESI-MS(+) m/z [M+2H]²⁺: 1142.9.

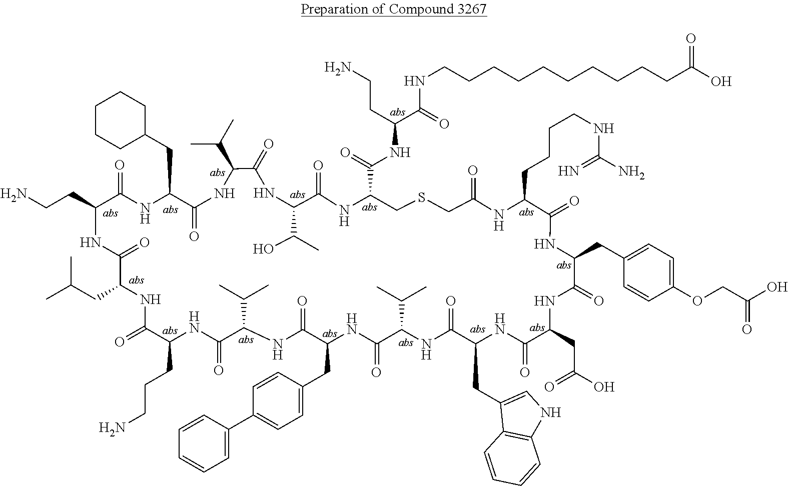 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](C(C)O)NC(=O)[C@H](C(C)C)NC(=O)[C@H](CC2CCCCC2)NC(=O)[C@H](CCN)NC1=O]

Compound 3267 was prepared on a 50 μmol scale. The yield of the product was 9.7 mg, and its estimated purity by LCMS analysis was 100%. Analysis condition A: Retention time=1.51 min; ESI-MS(+) m/z [M+2H]²⁺: 1120.2.

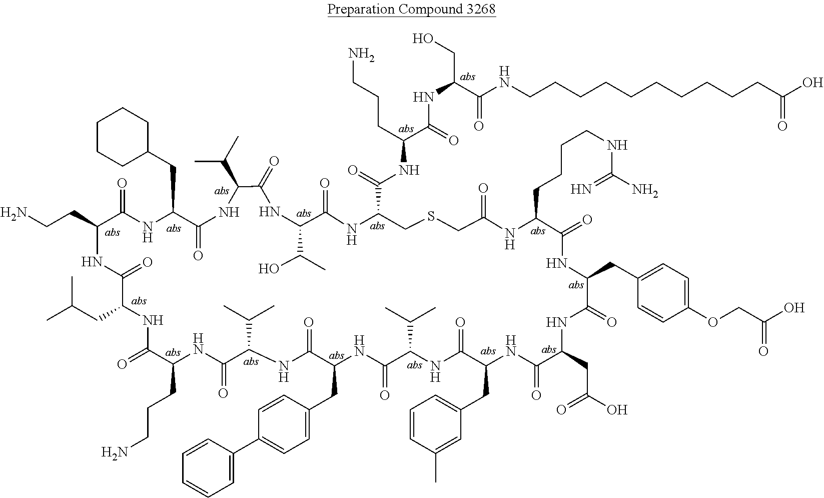 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CO)C(=O)NCCCCCCCCCCC(=O)O)NC(=O)C[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3268 was prepared on a 50 μmol scale. The yield of the product was 18.9 mg, and its estimated purity by LCMS analysis was 98.4%. Analysis condition A: Retention time-1.59 min; ESI-MS(+) m/z [M+3H]³⁺: 772.5.

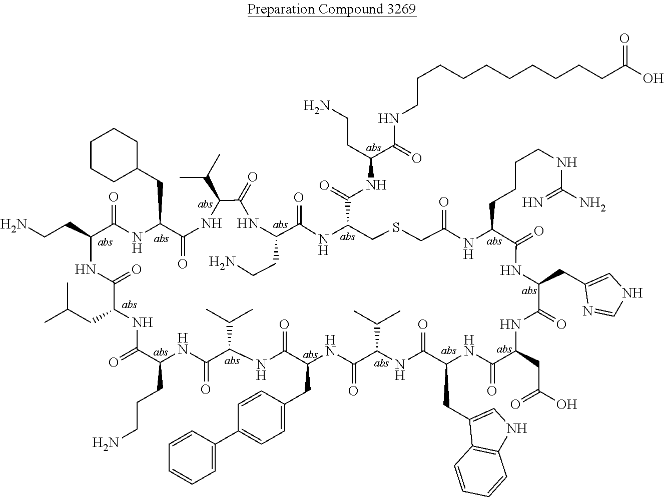 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]cn2)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CCN)NC(=O)[C@H](C(C)C)NC(=O)[C@H](CC2CCCCC2)NC(=O)[C@H](CCN)NC1=O]

Compound 3269 was prepared on a 50 μmol scale. The yield of the product was 15.5 mg, and its estimated purity by LCMS analysis was 100%. Analysis condition A: Retention time=1.52 min; ESI-MS(+) m/z [M+2H]²⁺: 1078.1.

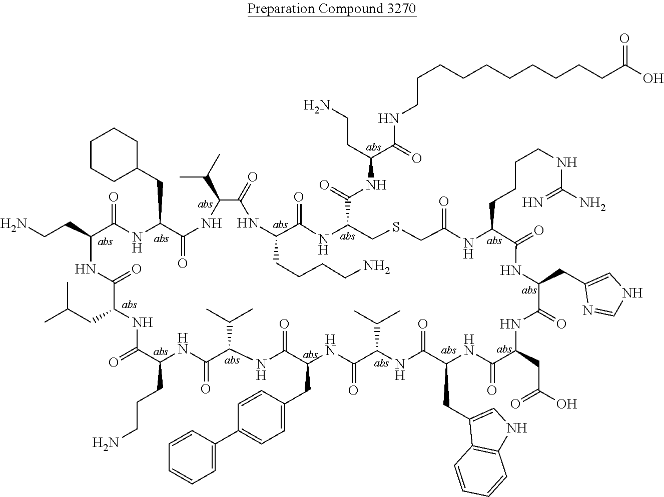 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]cn2)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CCCCN)NC(=O)[C@H](C(C)C)NC(=O)[C@H](CC2CCCCC2)NC(=O)[C@H](CCN)NC1=O]

Compound 3270 was prepared on a 50 μmol scale. The yield of the product was 22.3 mg, and its estimated purity by LCMS analysis was 100%. Analysis condition A: Retention time=1.57 min; ESI-MS(+) m/z [M+2H]²⁺: 1092.2.

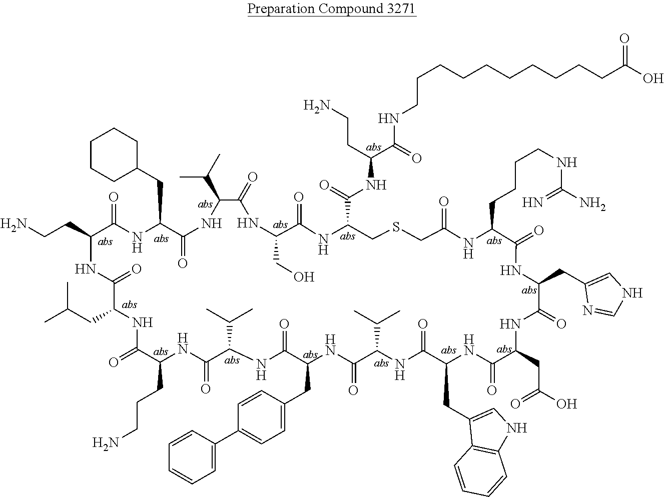 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]cn2)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CO)NC(=O)[C@H](C(C)C)NC(=O)[C@H](CC2CCCCC2)NC(=O)[C@H](CCN)NC1=O]

Compound 3271 was prepared on a 50 μmol scale. The yield of the product was 24.4 mg, and its estimated purity by LCMS analysis was 95.3%. Analysis condition A: Retention time=1.49 min; ESI-MS(+) m/z [M+2H]²⁺: 1072.

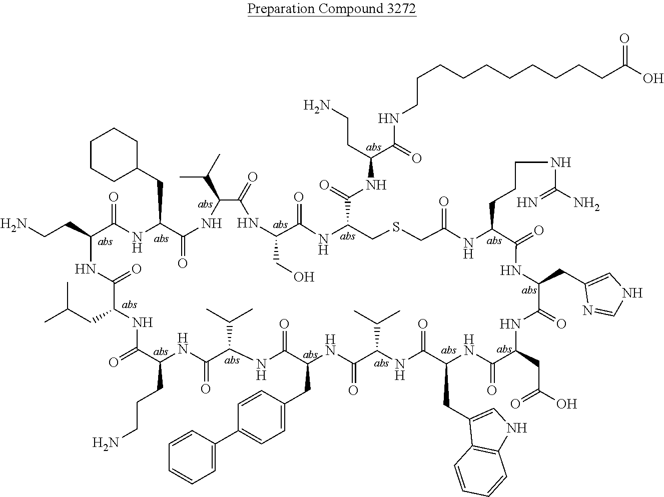 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]cn2)NC(=O)[C@H](CCCNC(=N)N)NC(=O)CSC[C@@H](C(=O)N[C@@H](CCN)C(=O)NCCCCCCCCCCC(=O)O)NC(=O)C[C@H](CO)NC(=O)[C@H](C(C)C)NC(=O)[C@H](CC2CCCCC2)NC(=O)[C@H](CCN)NC1=O]

Compound 3272 was prepared on a 50 μmol scale. The yield of the product was 20.6 mg, and its estimated purity by LCMS analysis was 95.5%. Analysis condition A: Retention time=1.51 min; ESI-MS(+) m/z [M+3H]³⁺: 710.1.

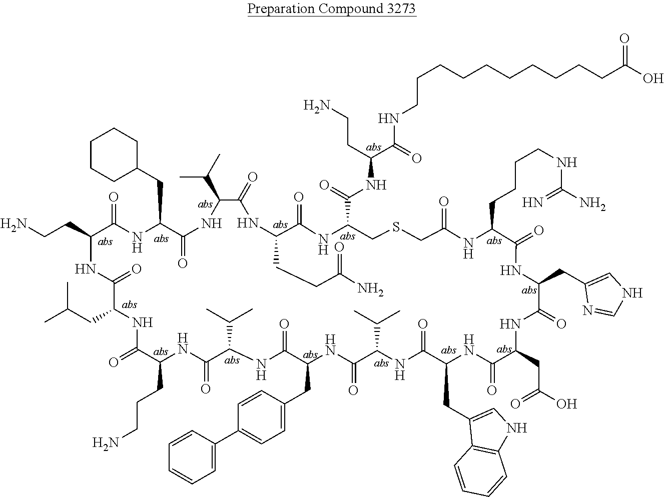 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]cn2)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CCC(N)=O)NC(=O)[C@H](C(C)C)NC(=O)[C@H](CC2CCCCC2)NC(=O)[C@H](CCN)NC1=O]

Compound 3273 was prepared on a 50 μmol scale. The yield of the product was 52.4 mg, and its estimated purity by LCMS analysis was 95.3%. Analysis condition A: Retention time=1.5 min; ESI-MS(+) m/z [M+2H]²⁺: 1092.2.

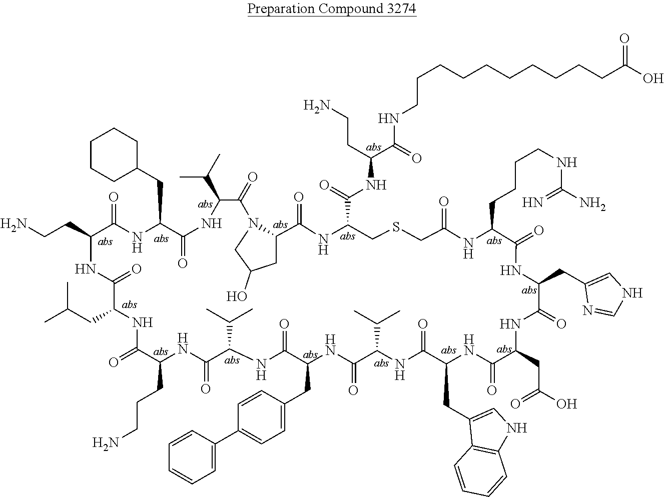

Compound 3274 was prepared on a 50 μmol scale. The yield of the product was 68.9 mg, and its estimated purity by LCMS analysis was 100%. Analysis condition A: Retention time-1.54 min; ESI-MS(+) m/z [M+3H]³⁺: 723.3.

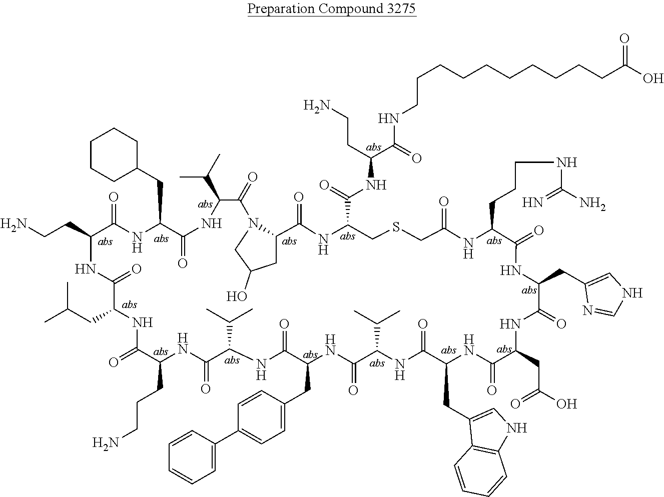

Compound 3275 was prepared on a 50 μmol scale. The yield of the product was 49.5 mg, and its estimated purity by LCMS analysis was 97.2%. Analysis condition B: Retention time=1.28 min; ESI-MS(+) m/z [M+3H]³⁺: 718.9.

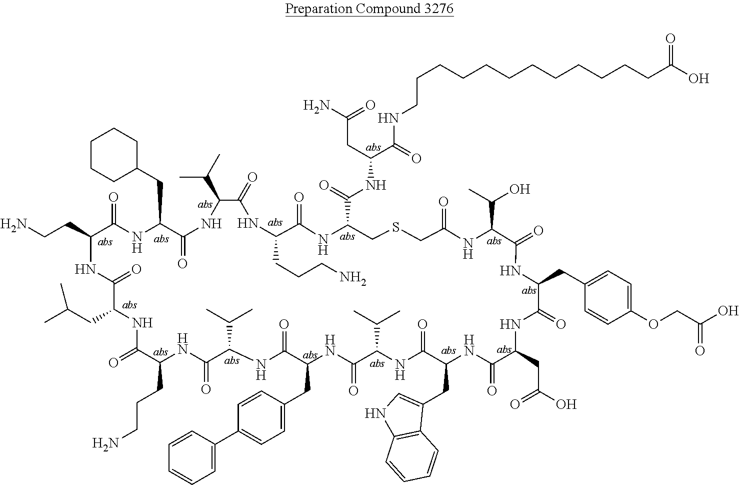 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Compound 3276 was prepared on a 50 μmol scale. The yield of the product was 30.5 mg, and its estimated purity by LCMS analysis was 99.4%. Analysis condition B: Retention time=1.57 min; ESI-MS(+) m/z [M+2H]²⁺: 1099.

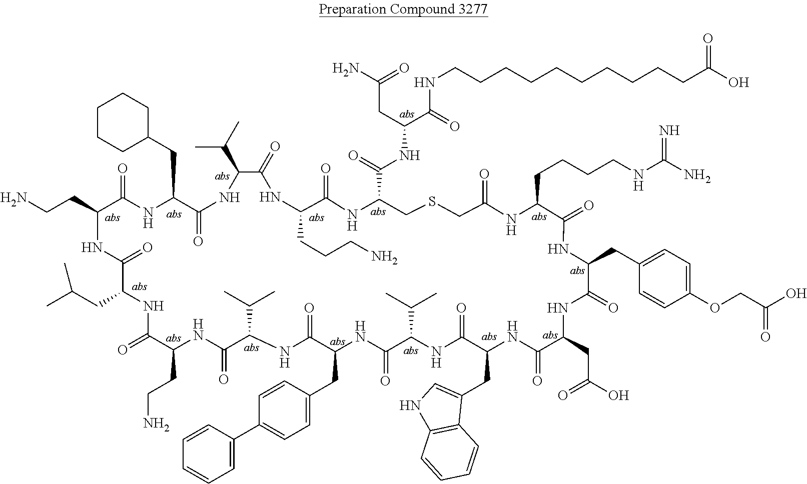 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@H](CC(N)=O)C(=O)NCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Compound 3277 was prepared on a 50 μmol scale. The yield of the product was 52.9 mg, and its estimated purity by LCMS analysis was 97.4%. Analysis condition B: Retention time=1.46 min; ESI-MS(+) m/z [M+2H]²⁺: 1127.

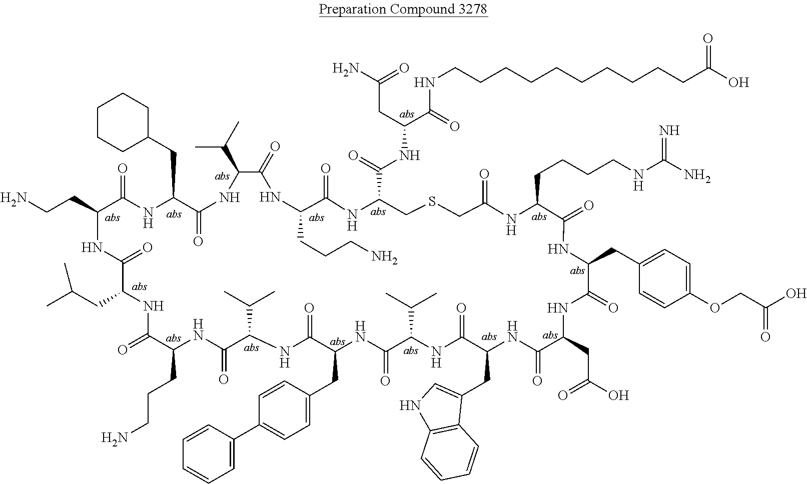 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@H](CC(N)=O)C(=O)NCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Compound 3278 was prepared on a 50 μmol scale. The yield of the product was 30.1 mg, and its estimated purity by LCMS analysis was 95.7%. Analysis condition B: Retention time=1.49 min; ESI-MS(+) m/z [M+2H]²⁺: 1133.8.

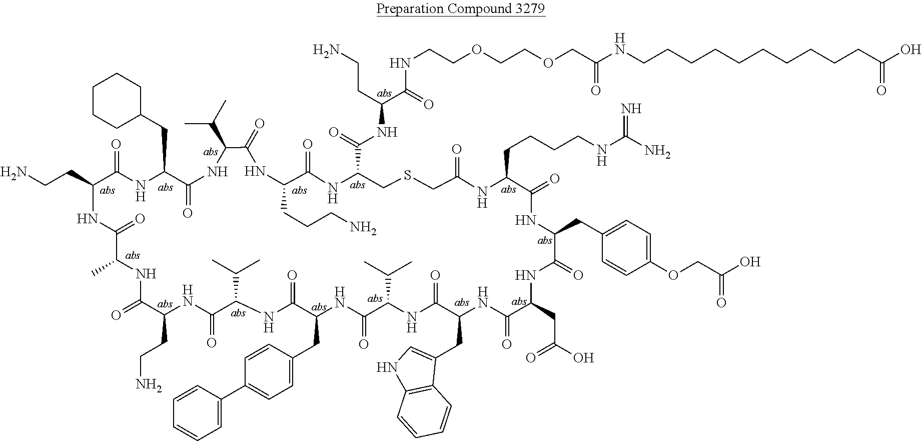 [SMILES: CC(C)[C@@H]1NC(=O)[C@H](CC2CCCCC2)NC(=O)[C@H](CCN)NC(=O)[C@@H](C)NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCOCCOCC(=O)NCCCCCCCCCCC(=O)O)NC(=O)C[C@H](CCCN)NC1=O]

Compound 3279 was prepared on a 50 μmol scale. The yield of the product was 51.6 mg, and its estimated purity by LCMS analysis was 96%. Analysis condition B: Retention time=1.39 min; ESI-MS(+) m/z [M+3H]³⁺: 781.1.

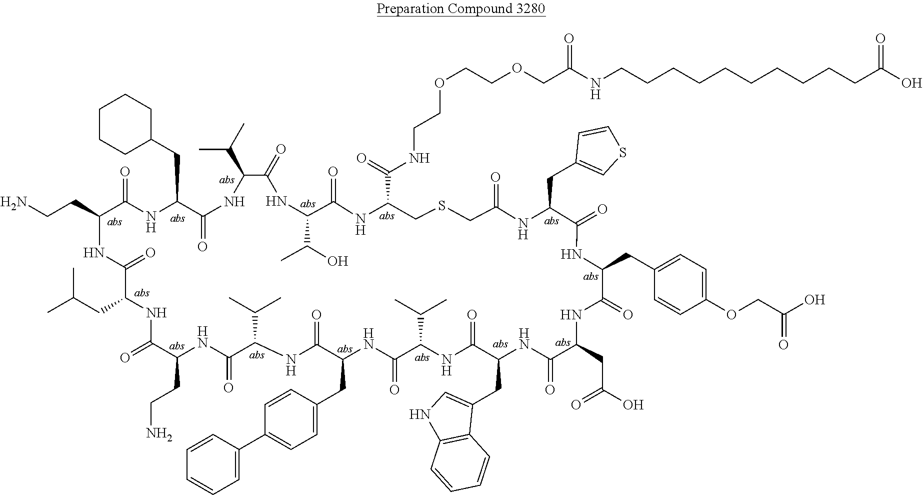 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)NCCCCCCCCCCC(=O)O)NC(=O)C[C@H](C(C)O)NC(=O)[C@H](C(C)C)NC(=O)[C@H](CC2CCCCC2)NC(=O)[C@H](CCN)NC1=O]

Compound 3280 was prepared on a 50 μmol scale. The yield of the product was 8.6 mg, and its estimated purity by LCMS analysis was 95.2%. Analysis condition A: Retention time=1.83 min; ESI-MS(+) m/z [M+2H]²⁺: 1127.3.

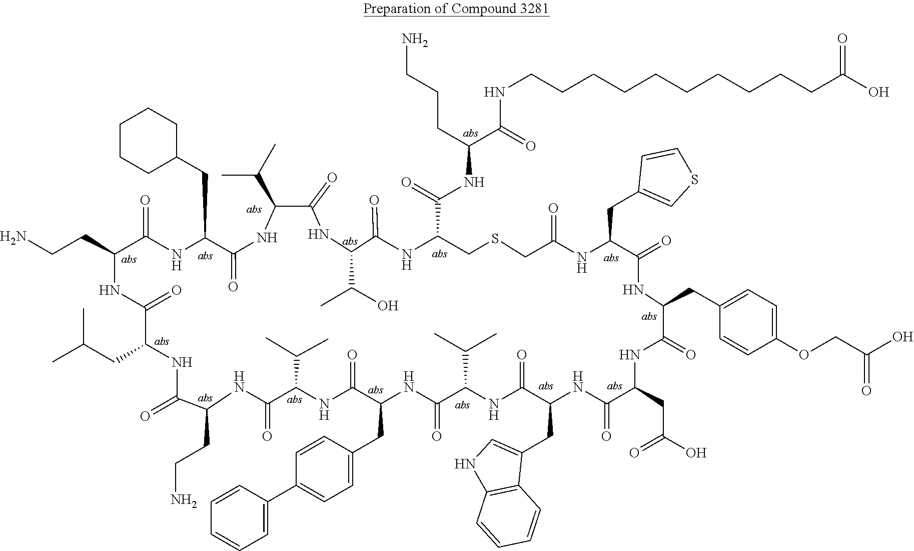 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3281 was prepared on a 50 μmol scale. The yield of the product was 4.2 mg, and its estimated purity by LCMS analysis was 96.7%. Analysis condition B: Retention time=1.71 min; ESI-MS(+) m/z [M+2H]²⁺: 1112.2.

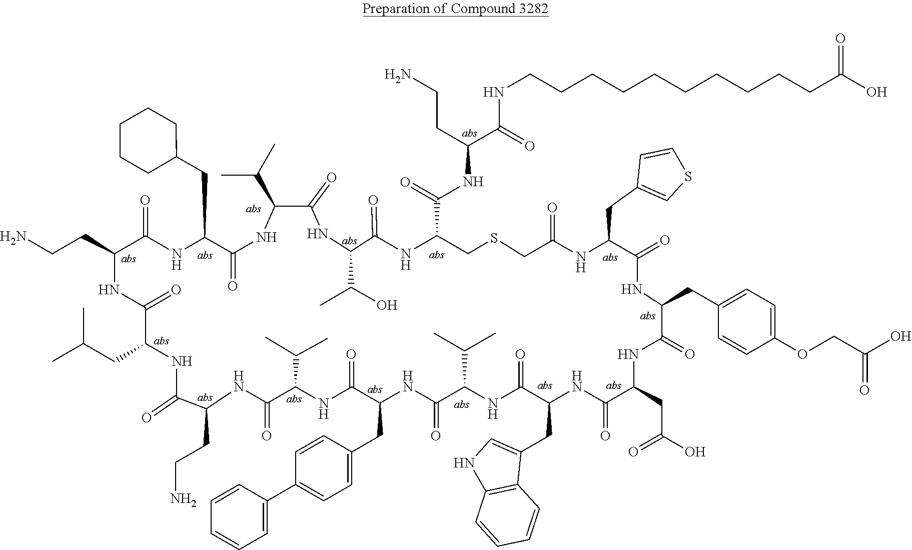 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3282 was prepared on a 50 μmol scale. The yield of the product was 4.1 mg, and its estimated purity by LCMS analysis was 100%. Analysis condition B: Retention time=1.7 min; ESI-MS(+) m/z [M+2H]²⁺: 1105.2.

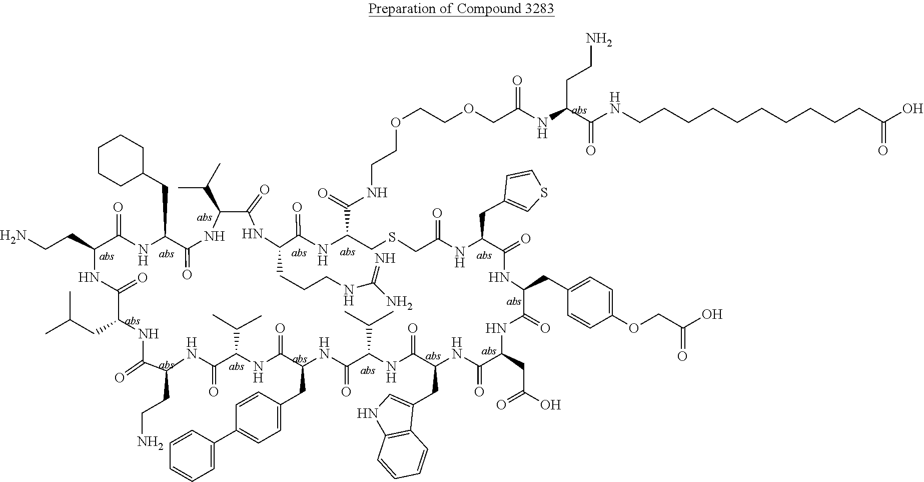 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)N[C@@H](CCN)C(=O)NCCCCCCCCCCC(=O)O)NC(=O)[C@H](CCCNC(=N)N)NC(=O)[C@H](C(C)C)NC(=O)[C@H](CC2CCCCC2)NC(=O)[C@H](CCN)NC1=O]

Compound 3283 was prepared on a 50 μmol scale. The yield of the product was 18.2 mg, and its estimated purity by LCMS analysis was 100%. Analysis condition A: Retention time=1.7 min; ESI-MS(+) m/z [M+2H]²⁺: 1205.1.

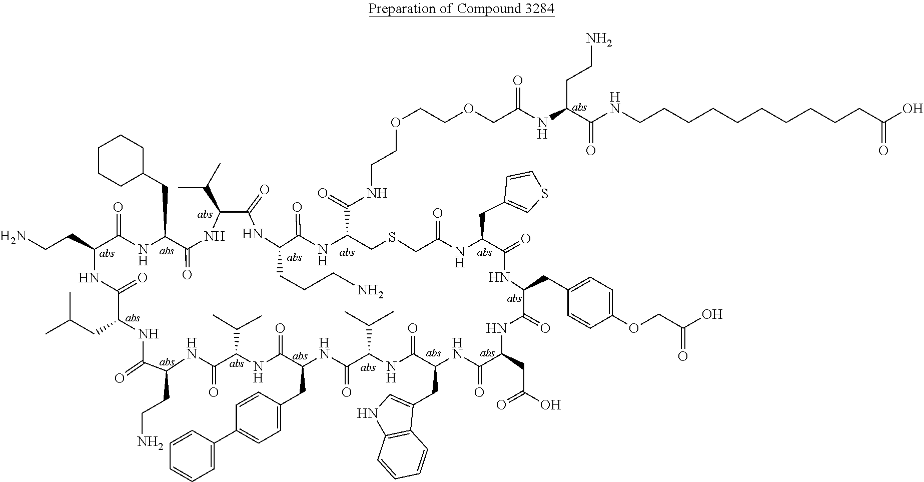 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)NCCOCCOCC(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 3284 was prepared on a 50 μmol scale. The yield of the product was 18.5 mg, and its estimated purity by LCMS analysis was 95.2%. Analysis condition B: Retention time=1.47, 1.52 min; ESI-MS(+) m/z [M+3H]³⁺: 790.04, 790.04.

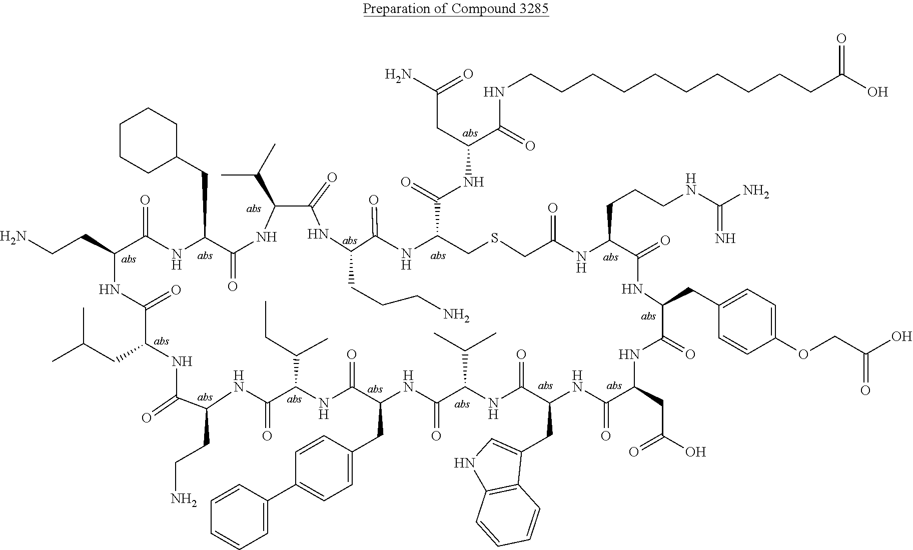 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCN)NC1=O]

Compound 3285 was prepared on a 50 μmol scale. The yield of the product was 25.8 mg, and its estimated purity by LCMS analysis was 95.4%. Analysis condition B: Retention time=1.38 min; ESI-MS(+) m/z [M+2H]²⁺: 1127.2.

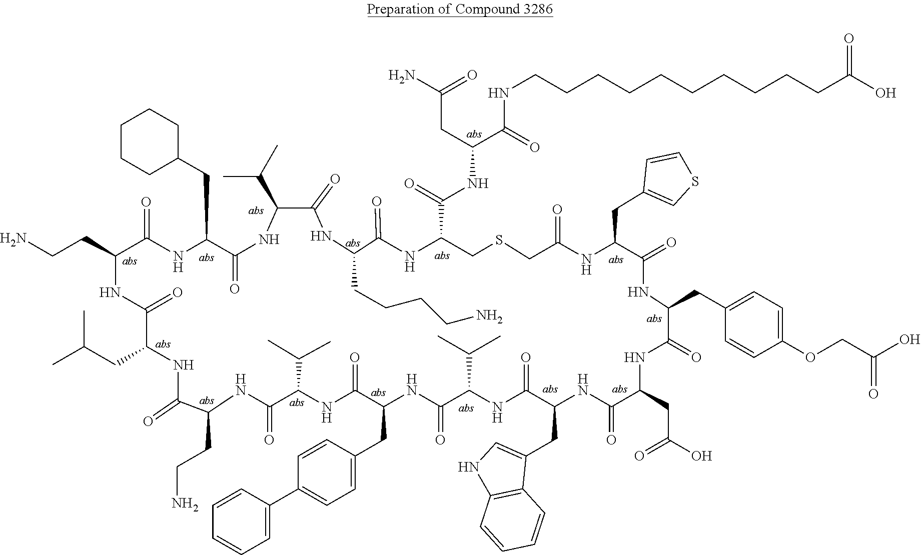 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@H](CC(N)=O)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 3286 was prepared on a 50 μmol scale. The yield of the product was 54.6 mg, and its estimated purity by LCMS analysis was 94.9%. Analysis condition A: Retention time=1.7 min; ESI-MS(+) m/z [M+3H]³⁺: 750.9.

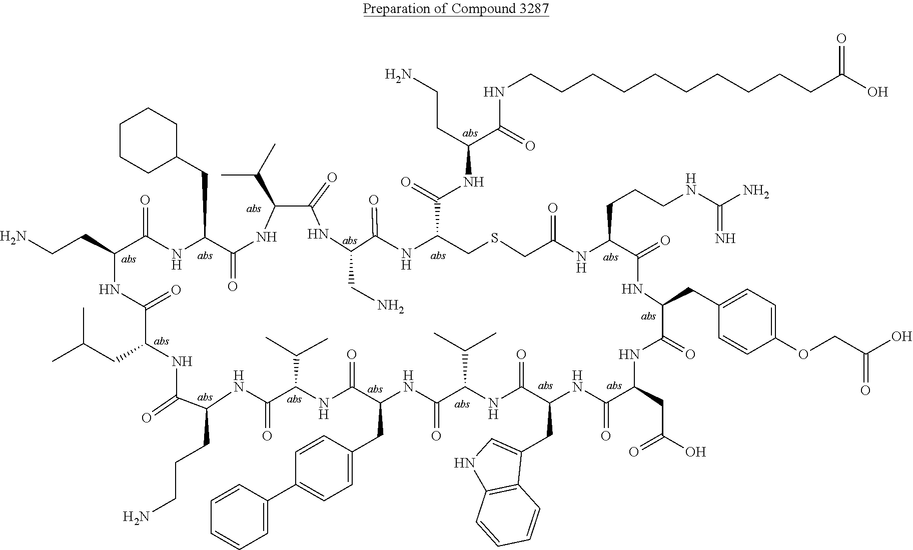 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3287 was prepared on a 50 μmol scale. The yield of the product was 28.2 mg, and its estimated purity by LCMS analysis was 95.7%. Analysis condition A: Retention time=1.53 min; ESI-MS(+) m/z [M+2H]²⁺: 1106.2.

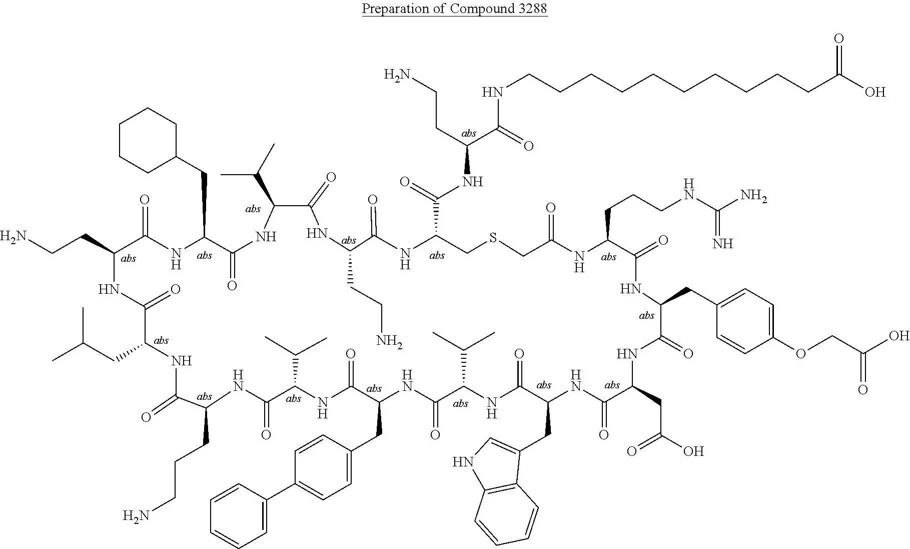 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCN)NC(=O)[C@H](C(C)C)NC(=O)[C@H](CC2CCCCC2)NC(=O)[C@H](CCN)NC1=O]

Compound 3288 was prepared on a 50 μmol scale. The yield of the product was 11.2 mg, and its estimated purity by LCMS analysis was 90%. Analysis condition A: Retention time=1.5 min; ESI-MS(+) m/z [M+2H]²⁺: 1113.2.

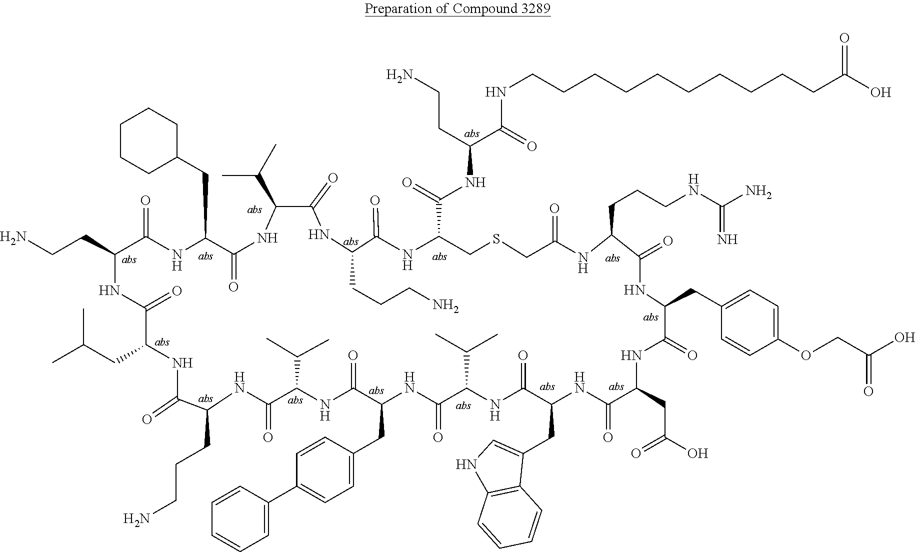 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 3289 was prepared on a 50 μmol scale. The yield of the product was 22.7 mg, and its estimated purity by LCMS analysis was 100%. Analysis condition A: Retention time=1.78 min; ESI-MS(+) m/z [M+3H]³⁺: 747.

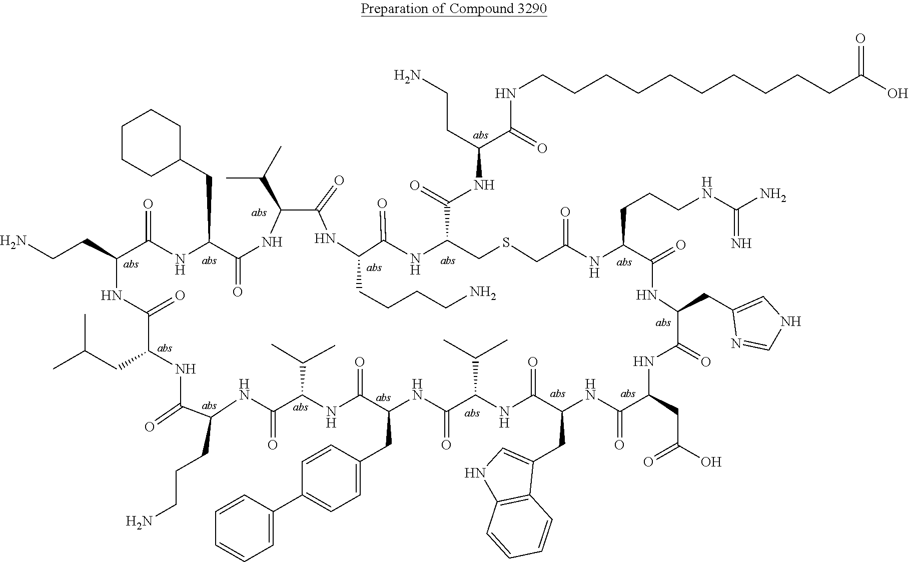 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]cn2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC2CCCCC2)NC(=O)[C@H](CCN)NC1=O]

Compound 3290 was prepared on a 50 μmol scale. The yield of the product was 17.1 mg, and its estimated purity by LCMS analysis was 98.4%. Analysis condition A: Retention time=1.47 min; ESI-MS(+) m/z [M+2H]²⁺: 1085.2.

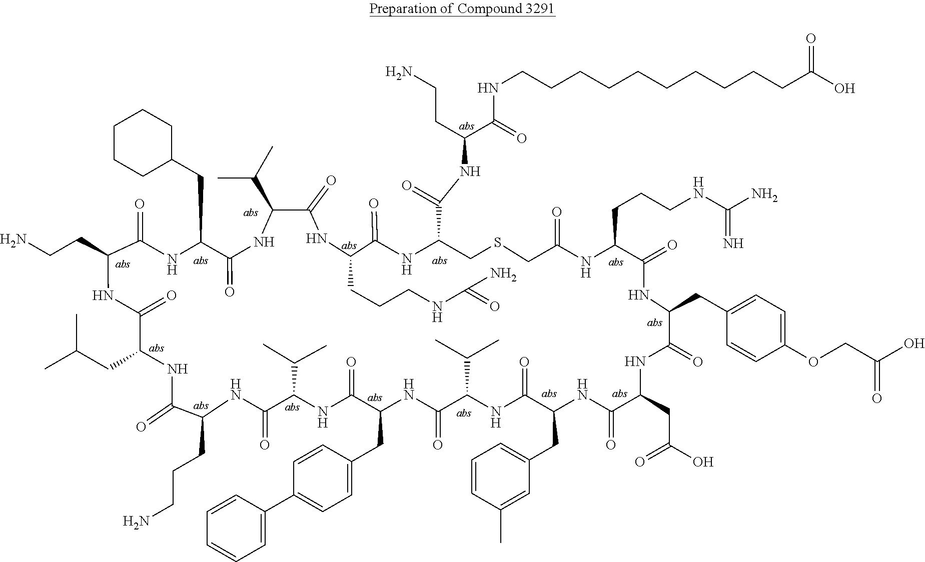 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3291 was prepared on a 50 μmol scale. The yield of the product was 8.1 mg, and its estimated purity by LCMS analysis was 98.9%. Analysis condition B: Retention time=1.56 min; ESI-MS(+) m/z [M+2H]²⁺: 1129.1.

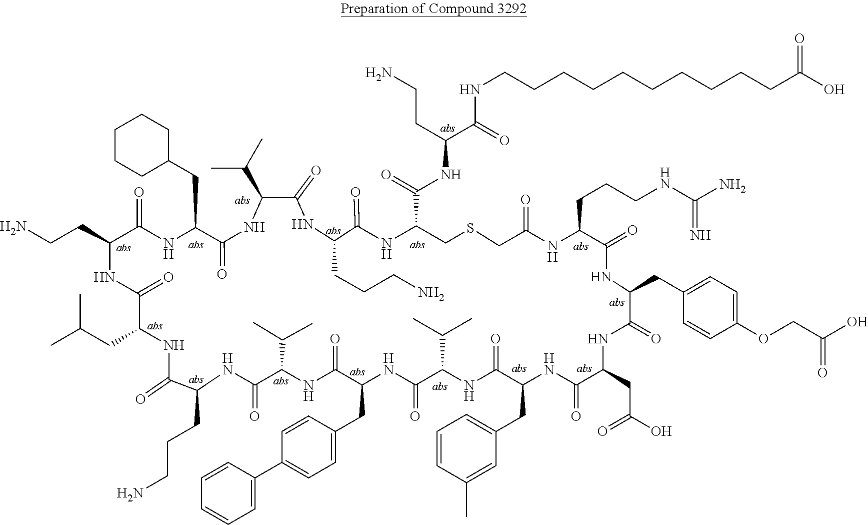 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3292 was prepared on a 50 μmol scale. The yield of the product was 14.5 mg, and its estimated purity by LCMS analysis was 95.9%. Analysis condition A: Retention time=1.62 min; ESI-MS(+) m/z [M+3H]³⁺: 739.

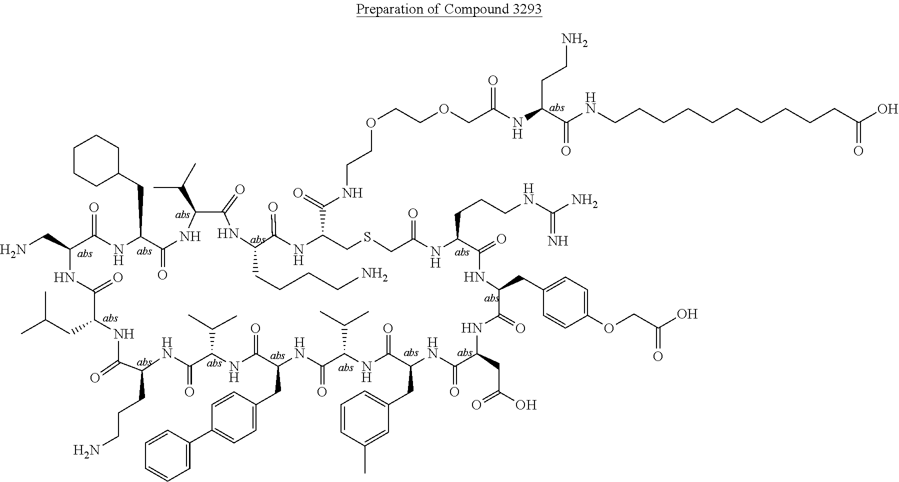 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3293 was prepared on a 50 μmol scale. The yield of the product was 29.1 mg, and its estimated purity by LCMS analysis was 100%. Analysis condition B: Retention time=1.43 min; ESI-MS(+) m/z [M+3H]³⁺: 787.2.

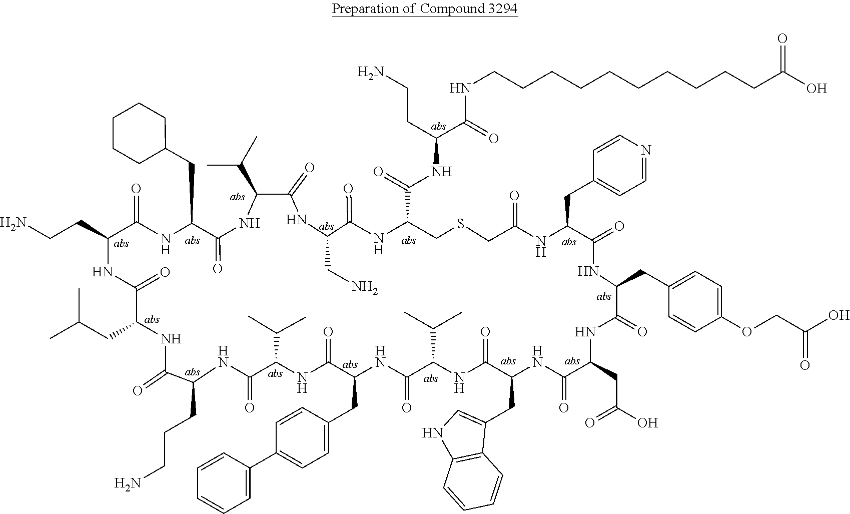 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(=O)NCCCCCCCCCCC(=O)O)NC(=O)[C@H](CN)NC(=O)[C@H](C(C)C)NC(=O)[C@H](CC2CCCCC2)NC(=O)[C@H](CCN)NC1=O]

Compound 3294 was prepared on a 50 μmol scale. The yield of the product was 27.5 mg, and its estimated purity by LCMS analysis was 95.8%. Analysis condition A: Retention time=1.61 min; ESI-MS(+) m/z [M+2H]²⁺: 1102.2.

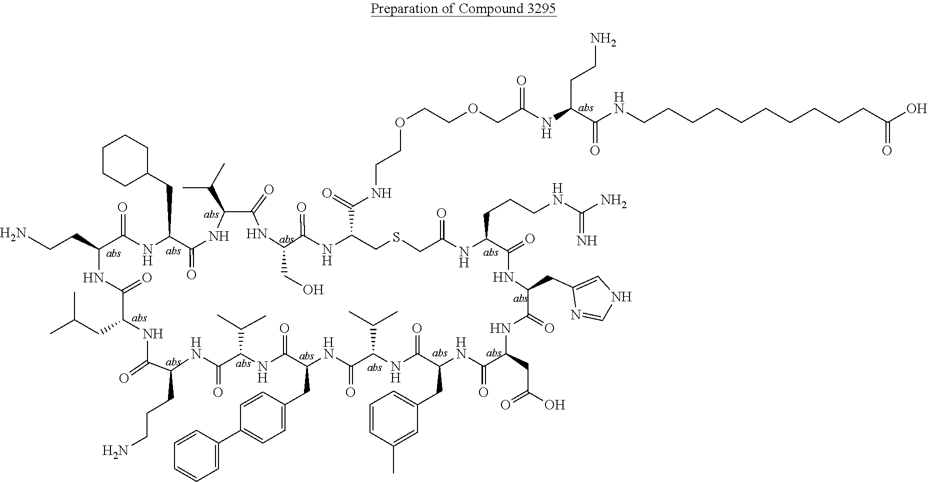 [SMILES: Cc1cccc(C[C@@H]2NC(=O)[C@H](CC(=O)O)NC(=O)[C@H](Cc3c[nH]cn3)NC(=O)[C@H](CCCNC(=N)N)NC(=O)CSC[C@@H](C(=O)NCCOCCOCC(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3295 was prepared on a 50 μmol scale. The yield of the product was 25.3 mg, and its estimated purity by LCMS analysis was 98.3%. Analysis condition B: Retention time=1.37 min; ESI-MS(+) m/z [M+3H]³⁺: 750.2.

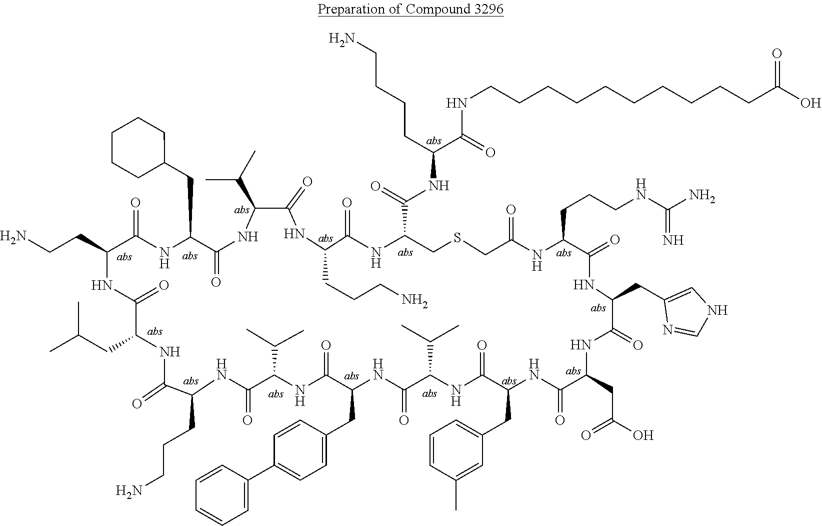 [SMILES: Cc1cccc(C[C@@H]2NC(=O)[C@H](CC(=O)O)NC(=O)[C@H](Cc3c[nH]cn3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3296 was prepared on a 50 μmol scale. The yield of the product was 39.5 mg, and its estimated purity by LCMS analysis was 98.6%. Analysis condition A: Retention time=1.51 min; ESI-MS(+) m/z [M+2H]²⁺: 1079.4.

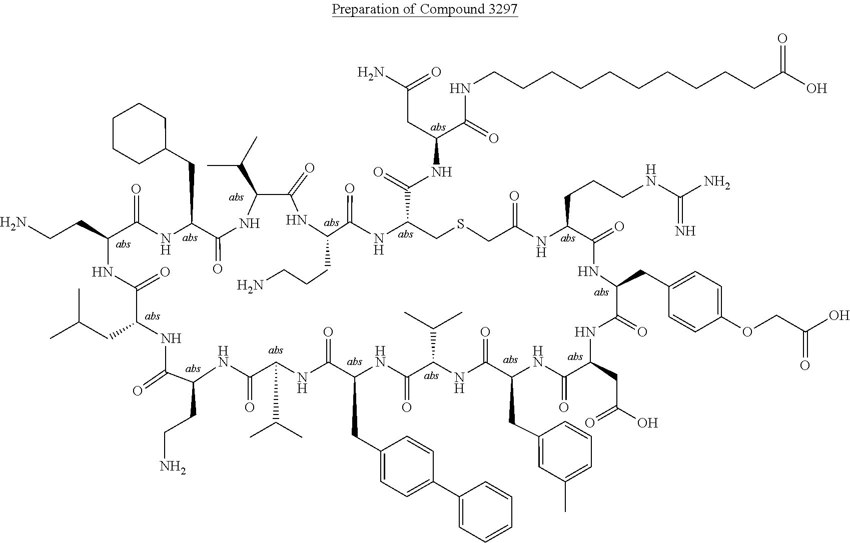 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CC(N)=O)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3297 was prepared on a 50 μmol scale. The yield of the product was 28 mg, and its estimated purity by LCMS analysis was 95.4%. Analysis condition B: Retention time=1.43 min; ESI-MS(+) m/z [M+3H]³⁺: 743.1.

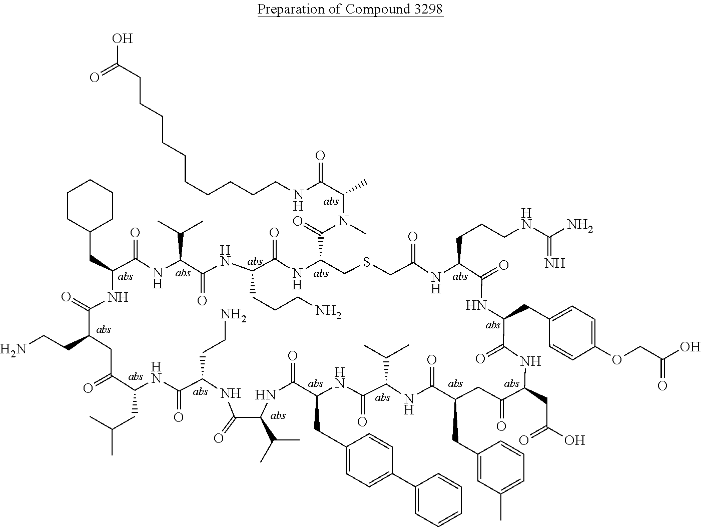 [SMILES: Cc1cccc(C[C@@H]2CC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N(C)[C@@H](C)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)CC(=O)C[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3298 was prepared on a 50 μmol scale. The yield of the product was 23.5 mg, and its estimated purity by LCMS analysis was 97.1%. Analysis condition A: Retention time=1.47 min; ESI-MS(+) m/z [M+3H]³⁺: 729.1.

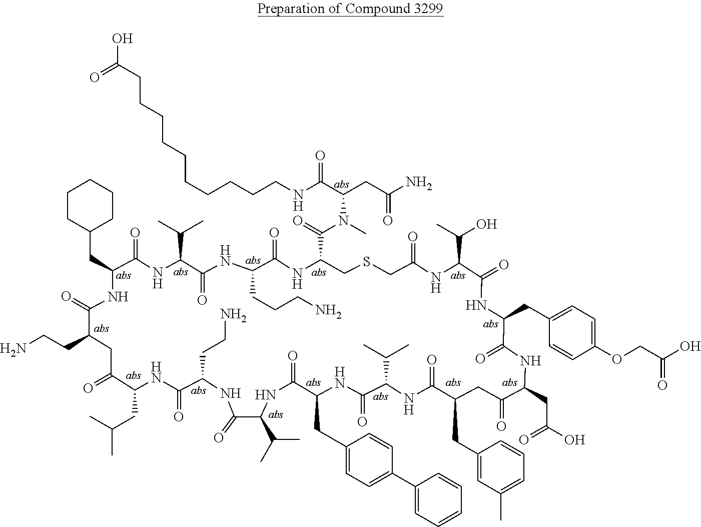 [SMILES: Cc1cccc(C[C@@H]2CC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)[C@@H](CC(N)=O)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)CC(=O)C[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3299 was prepared on a 50 μmol scale. The yield of the product was 19.2 mg, and its estimated purity by LCMS analysis was 97.8%. Analysis condition A: Retention time=1.52 min; ESI-MS(+) m/z [M+3H]³⁺: 724.8.

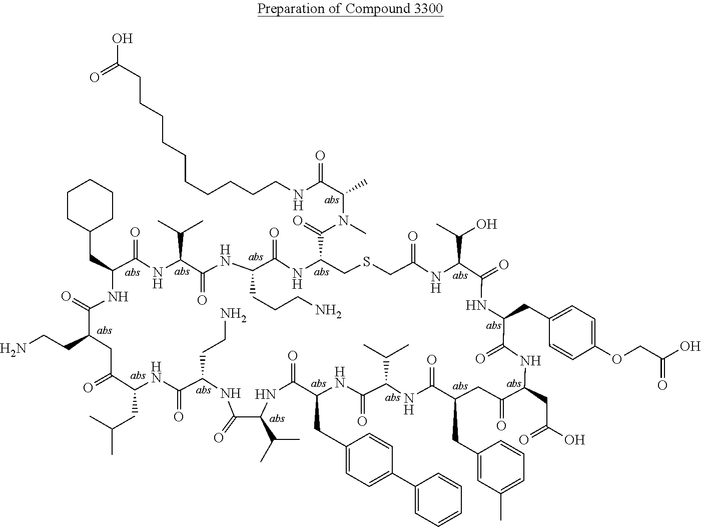 [SMILES: Cc1cccc(C[C@@H]2CC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)[C@@H](C)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)CC(=O)C[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3300 was prepared on a 50 μmol scale. The yield of the product was 30 mg, and its estimated purity by LCMS analysis was 97.2%. Analysis condition B: Retention time=1.56 min; ESI-MS(+) m/z [M+2H]²⁺: 1065.

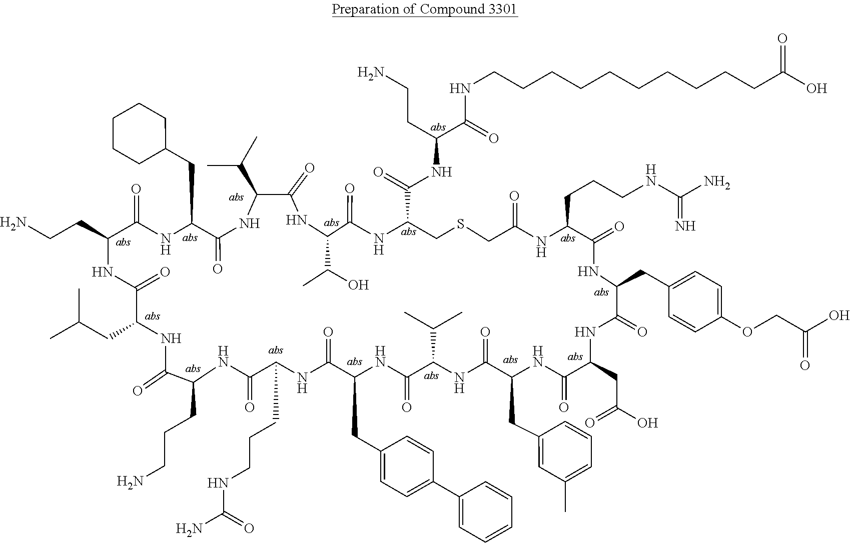 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3301 was prepared on a 50 μmol scale. The yield of the product was 19.6 mg, and its estimated purity by LCMS analysis was 95%. Analysis condition B: Retention time=1.49 min; ESI-MS(+) m/z [M+3H]³⁺: 754.

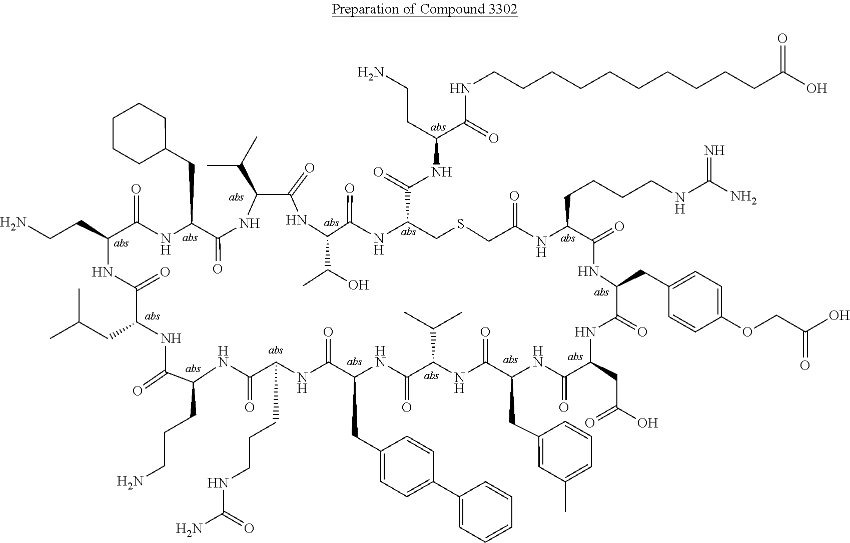 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3302 was prepared on a 50 μmol scale. The yield of the product was 19.3 mg, and its estimated purity by LCMS analysis was 97%. Analysis condition B: Retention time=1.45 min; ESI-MS(+) m/z [M+3H]³⁺: 758.2.

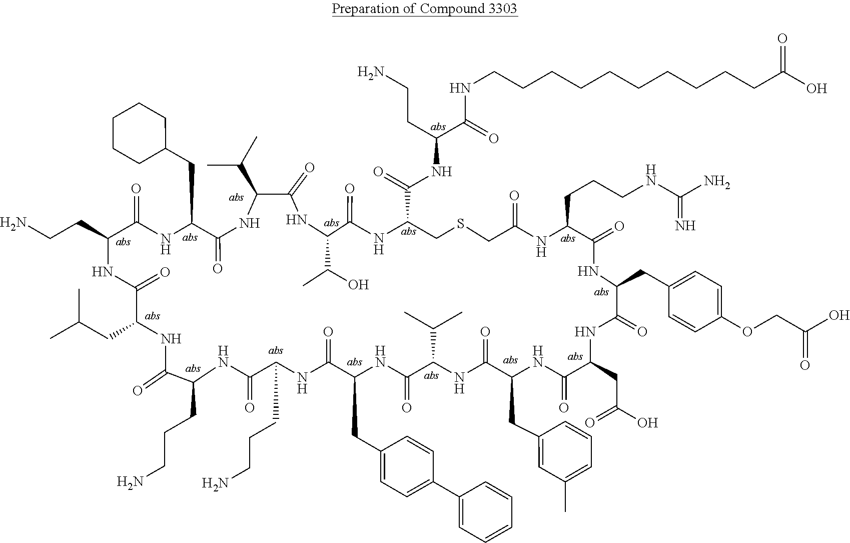 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCN)NC(=O)[C@H](Cc3ccc(-c4ccccc4)cc3)NC(=O)[C@H](C(C)C)NC2=O)c1]

Compound 3303 was prepared on a 50 μmol scale. The yield of the product was 31 mg, and its estimated purity by LCMS analysis was 100%. Analysis condition A: Retention time-1.47 min; ESI-MS(+) m/z [M+3H]³⁺: 739.3.

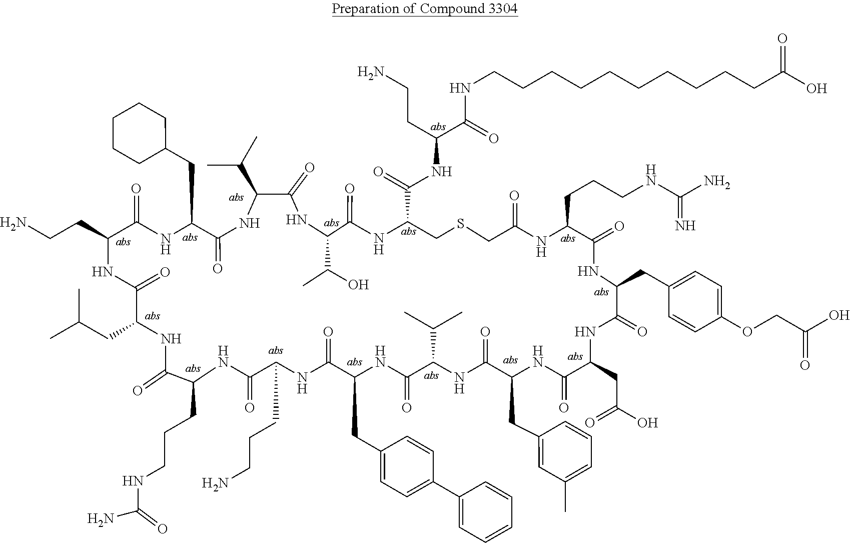 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](CCCN)NC(=O)[C@H](Cc3ccc(-c4ccccc4)cc3)NC(=O)[C@H](C(C)C)NC2=O)c1]

Compound 3304 was prepared on a 50 μmol scale. The yield of the product was 24.6 mg, and its estimated purity by LCMS analysis was 97.8%. Analysis condition B: Retention time=1.5 min; ESI-MS(+) m/z [M+3H]³⁺: 753.9.

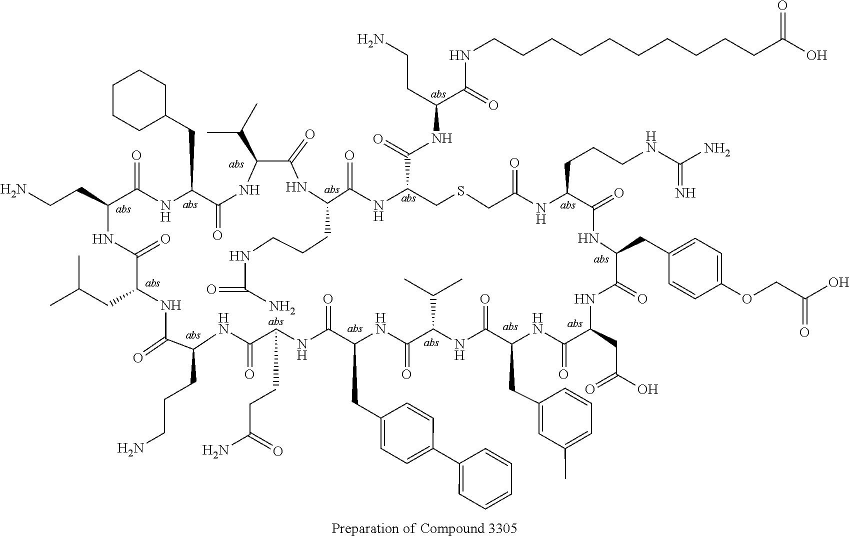 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 3305 was prepared on a 50 μmol scale. The yield of the product was 36.8 mg, and its estimated purity by LCMS analysis was 95.9%. Analysis condition A: Retention time=1.55 min; ESI-MS(+) m/z [M+3H]³⁺: 763.1.

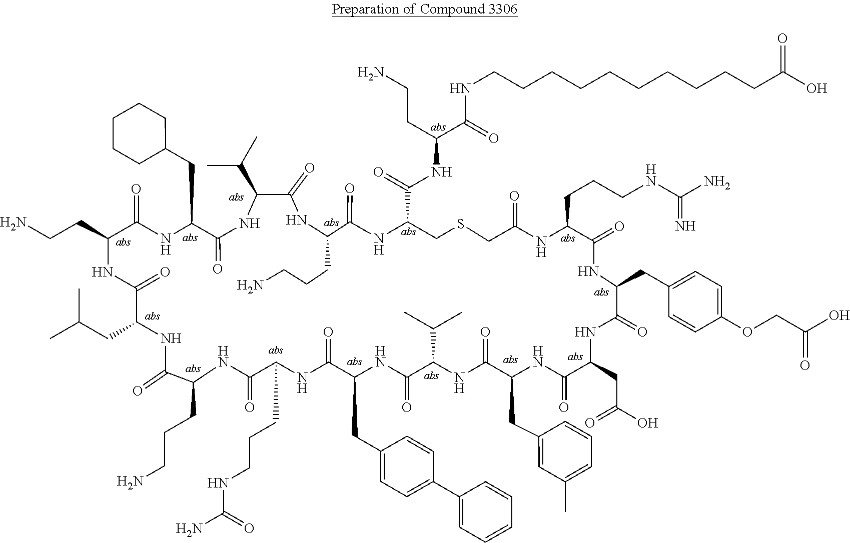 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3306 was prepared on a 50 μmol scale. The yield of the product was 14.1 mg, and its estimated purity by LCMS analysis was 96.8%. Analysis condition B: Retention time=1.38 min; ESI-MS(+) m/z [M+3H]³⁺: 758.1.

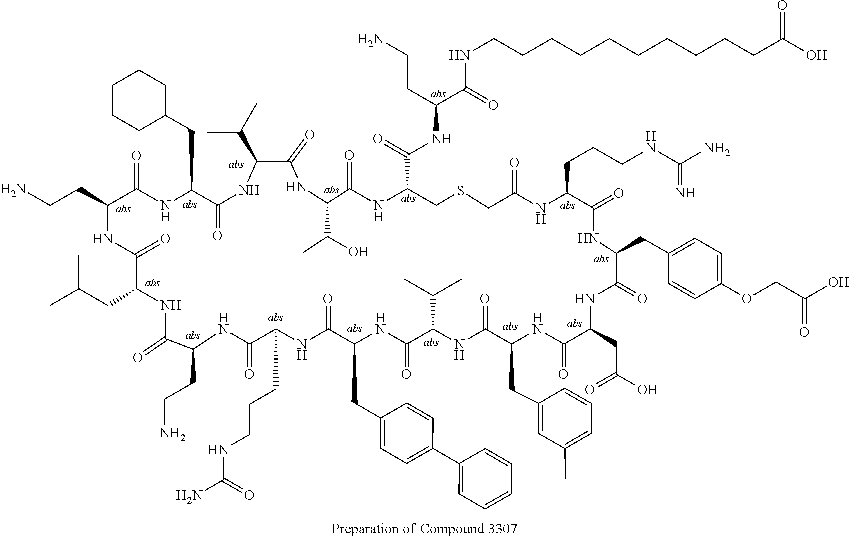 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 3307 was prepared on a 50 μmol scale. The yield of the product was 26.5 mg, and its estimated purity by LCMS analysis was 95%. Analysis condition B: Retention time=1.46 min; ESI-MS(+) m/z [M+3H]³⁺: 749.1.

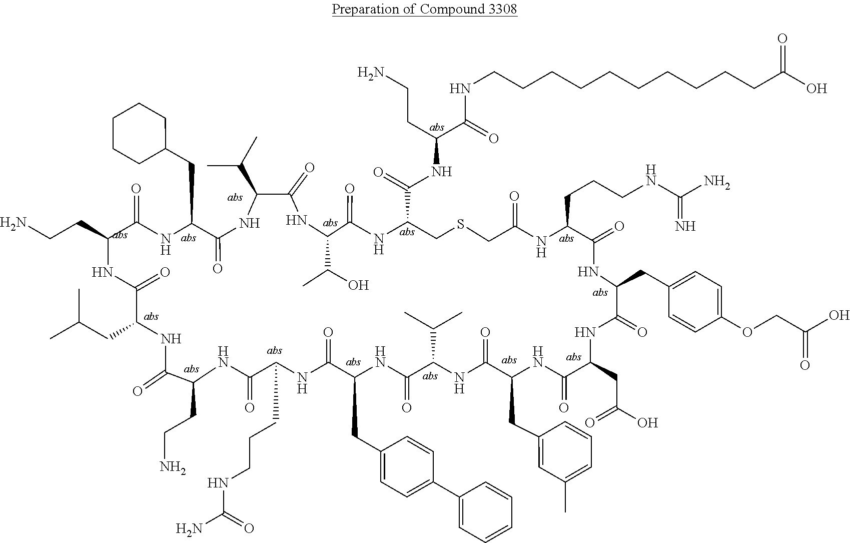 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCCNC(N)=O)NC(=O)[C@H](Cc3ccc(-c4ccccc4)cc3)NC(=O)[C@H](C(C)C)NC2=O)c1]

Compound 3308 was prepared on a 50 μmol scale. The yield of the product was 18.8 mg, and its estimated purity by LCMS analysis was 99.1%. Analysis condition A: Retention time-1.38 min; ESI-MS(+) m/z [M+3H]³⁺: 753.1.

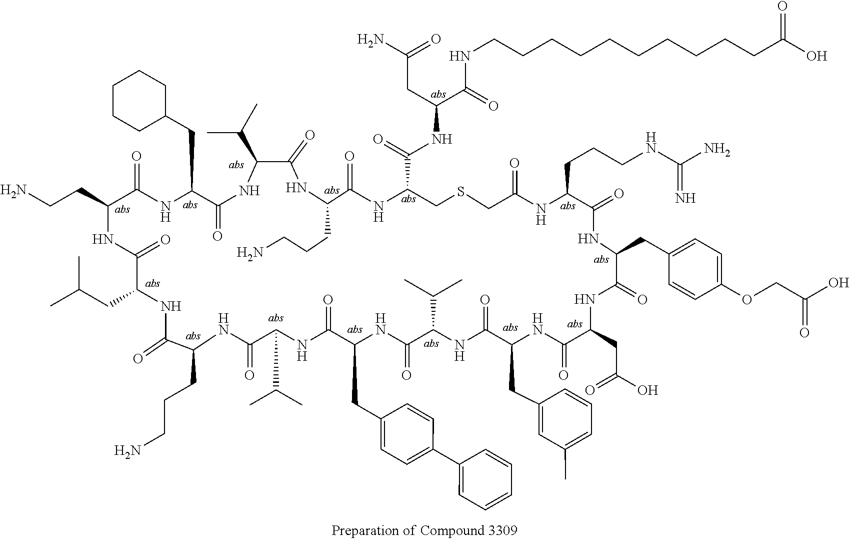 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CC(N)=O)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3309 was prepared on a 50 μmol scale. The yield of the product was 3.1 mg, and its estimated purity by LCMS analysis was 99.3%. Analysis condition B: Retention time=1.62 min; ESI-MS(+) m/z [M+2H]²⁺: 1101.

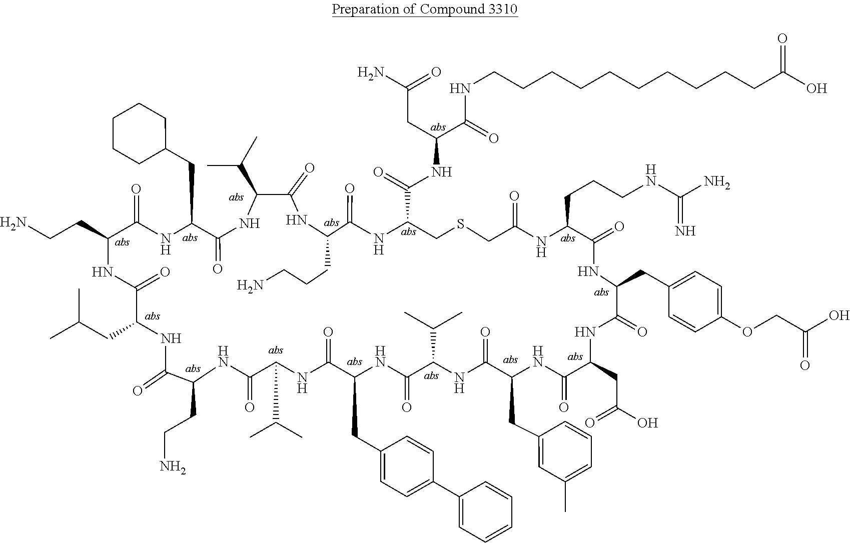 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CC(N)=O)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3310 was prepared on a 50 μmol scale. The yield of the product was 6.6 mg, and its estimated purity by LCMS analysis was 95.7%. Analysis condition B: Retention time-1.5 min; ESI-MS(+) m/z [M+2H]²⁺: 1094.1.

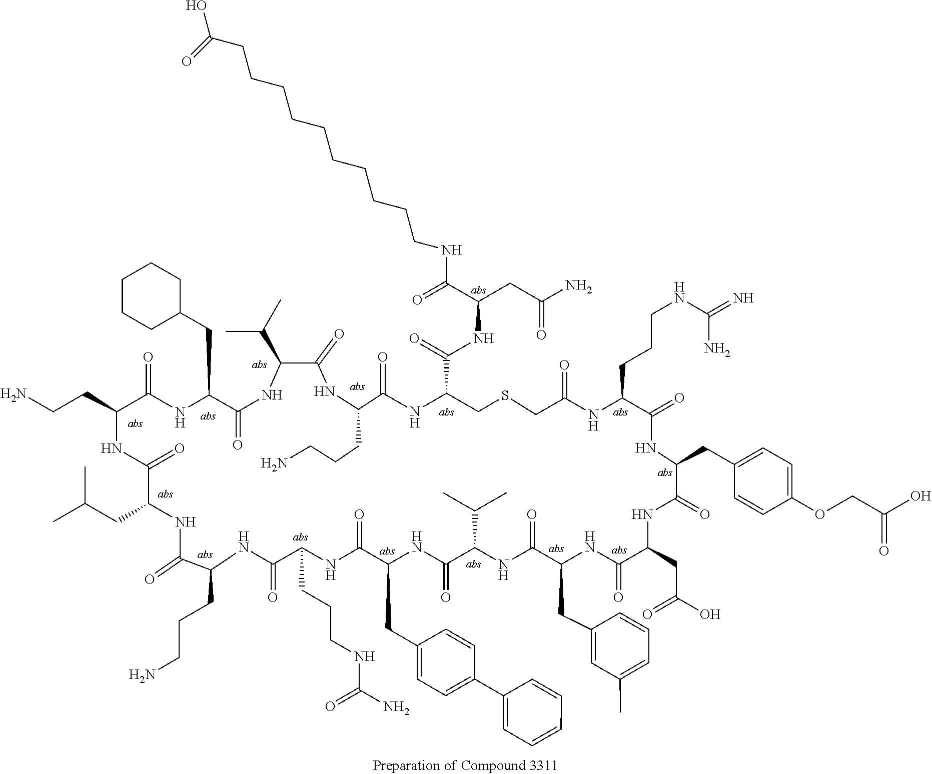 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3311 was prepared on a 50 μmol scale. The yield of the product was 13.4 mg, and its estimated purity by LCMS analysis was 90.4%. Analysis condition B: Retention time=1.54 min; ESI-MS(+) m/z [M+3H]³⁺: 763.

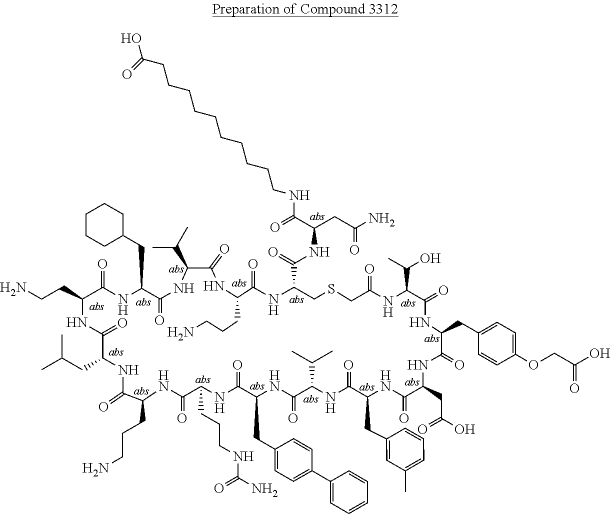 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3312 was prepared on a 50 μmol scale. The yield of the product was 8.2 mg, and its estimated purity by LCMS analysis was 100%. Analysis condition A: Retention time=1.6 min; ESI-MS(+) m/z [M+2H]²⁺: 1116.2.

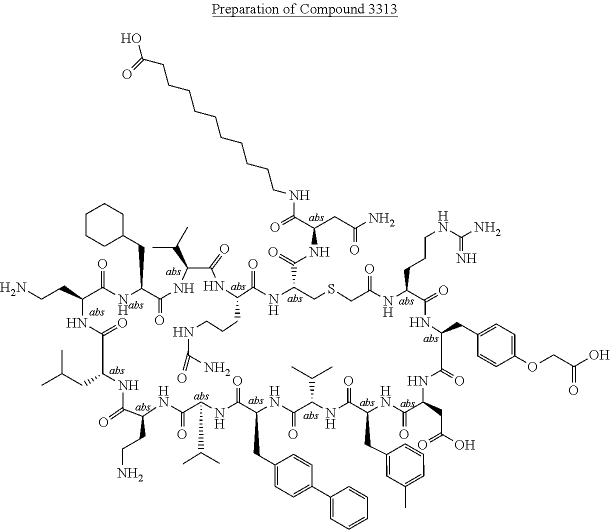 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3313 was prepared on a 50 μmol scale. The yield of the product was 11.7 mg, and its estimated purity by LCMS analysis was 100%. Analysis condition A: Retention time=1.5 min; ESI-MS(+) m/z [M+2H]²⁺: 1122.2.

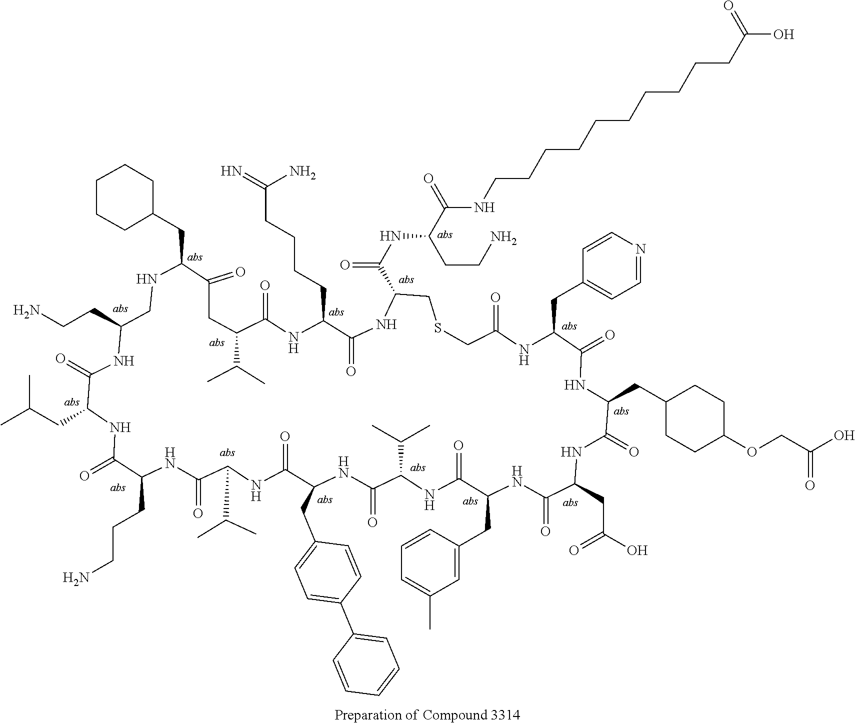 [SMILES: Cc1cccc(C[C@@H]2NC(=O)[C@H](CC(=O)O)NC(=O)[C@H](CC3CCC(OCC(=O)O)CC3)NC(=O)[C@H](Cc3ccncc3)NC(=O)CSC[C@@H](C(=O)N[C@@H](CCN)C(=O)NCCCCCCCCCCC(=O)O)NC(=O)[C@H](CCCCC(=N)N)NC(=O)[C@H](C(C)C)CC(=O)[C@H](CC3CCCCC3)NC[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3314 was prepared on a 50 μmol scale. The yield of the product was 14.4 mg, and its estimated purity by LCMS analysis was 95%. Analysis condition A: Retention time=1.61 min; ESI-MS(+) m/z [M+2H]²⁺: 1125.

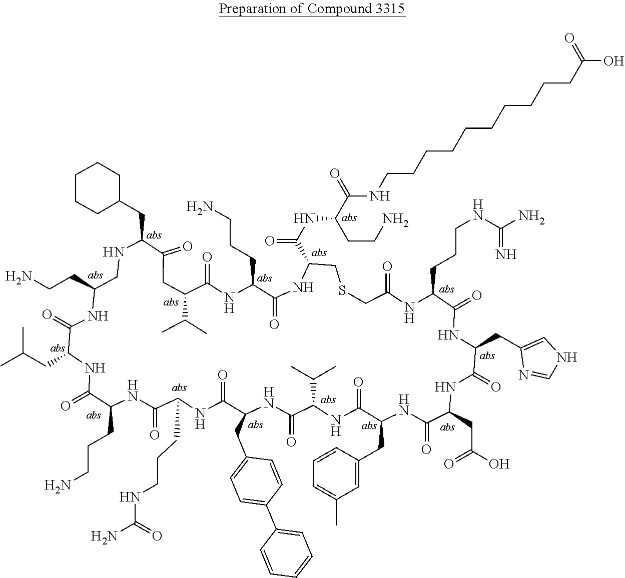 [SMILES: Cc1cccc(C[C@@H]2NC(=O)[C@H](CC(=O)O)NC(=O)[C@H](Cc3c[nH]cn3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCN)NC(=O)[C@H](C(C)C)CC(=O)[C@H](CC3CCCCC3)NC[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3315 was prepared on a 50 μmol scale. The yield of the product was 10.9 mg, and its estimated purity by LCMS analysis was 100%. Analysis condition A: Retention time-1.48 min; ESI-MS(+) m/z [M+3H]³⁺: 730.1.

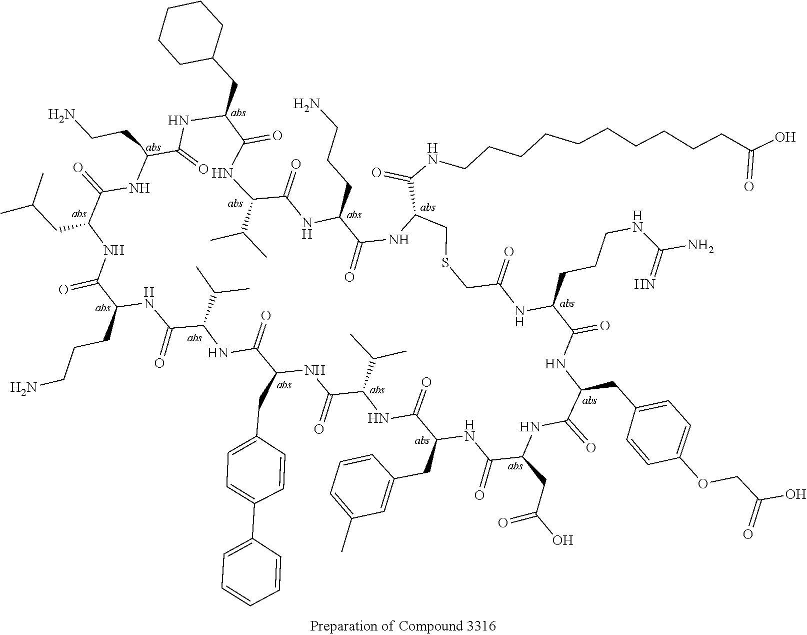

Compound 3316 was prepared on a 50 μmol scale. The yield of the product was 6 mg, and its estimated purity by LCMS analysis was 98.2%. Analysis condition A: Retention time=1.61 min; ESI-MS(+) m/z [M+3H]³⁺: 705.1.

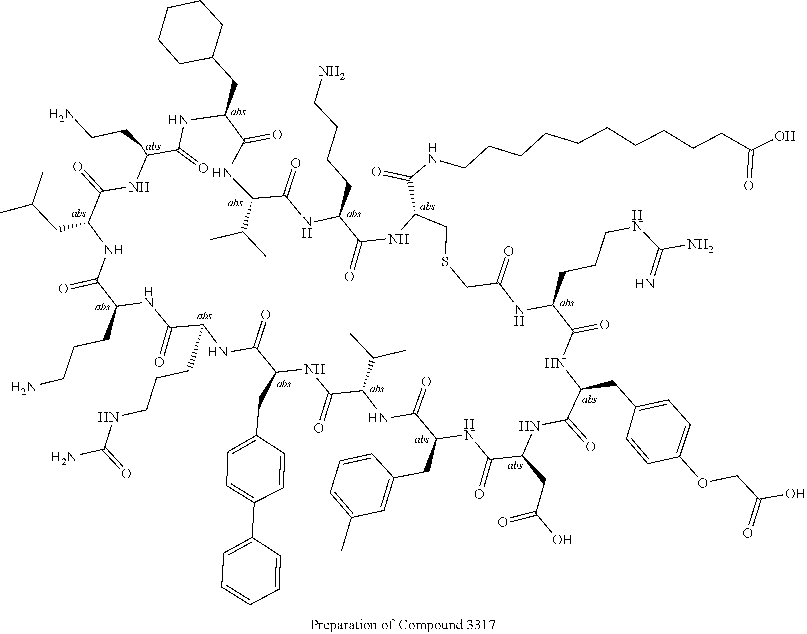

Compound 3317 was prepared on a 50 μmol scale. The yield of the product was 20.4 mg, and its estimated purity by LCMS analysis was 91.9%. Analysis condition B: Retention time=1.54 min; ESI-MS(+) m/z [M+2H]²⁺: 1094.1.

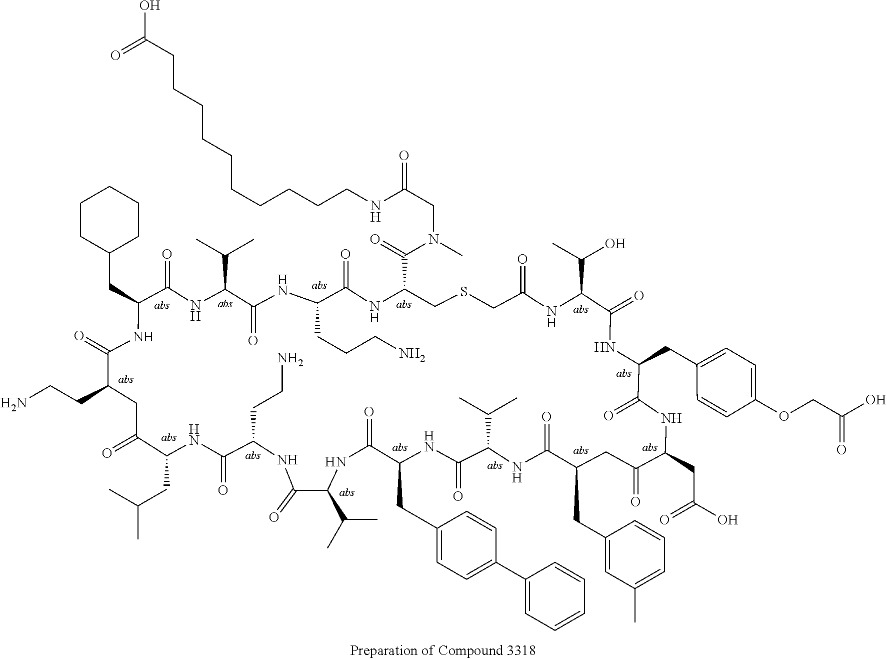 [SMILES: Cc1cccc(C[C@@H]2CC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)CC(=O)C[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3318 was prepared on a 50 μmol scale. The yield of the product was 23 mg, and its estimated purity by LCMS analysis was 95.7%. Analysis condition A: Retention time=1.62 min; ESI-MS(+) m/z [M+3H]³⁺: 706.1.

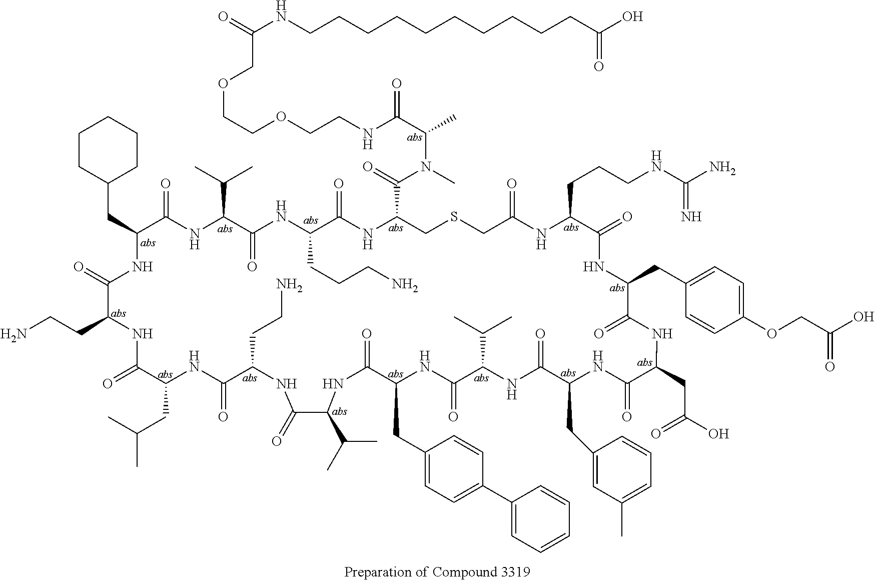 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N(C)[C@@H](C)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3319 was prepared on a 50 μmol scale. The yield of the product was 10.1 mg, and its estimated purity by LCMS analysis was 97.4%. Analysis condition B: Retention time=1.5 min; ESI-MS(+) m/z [M+3H]³⁺: 777.1.

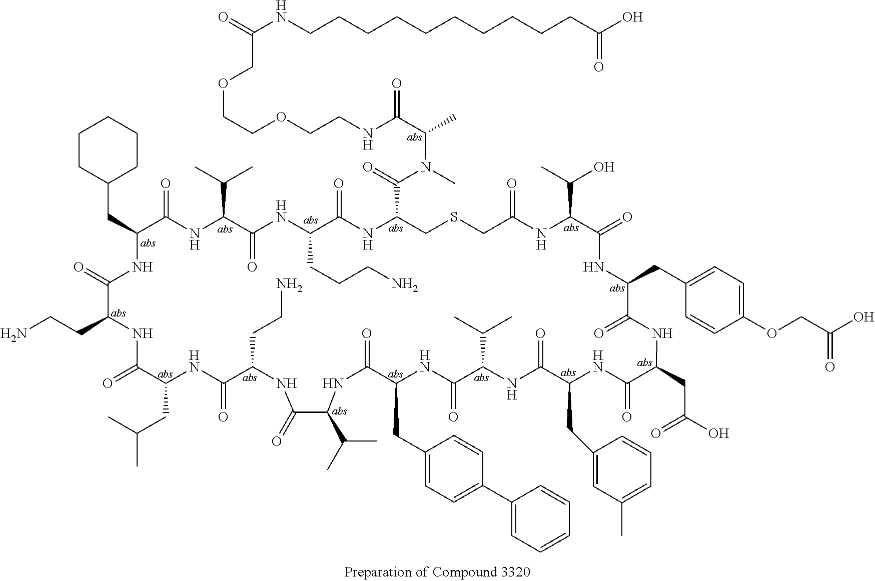 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)[C@@H](C)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3320 was prepared on a 50 μmol scale. The yield of the product was 5 mg, and its estimated purity by LCMS analysis was 92.1%. Analysis condition B: Retention time=1.71 min; ESI-MS(+) m/z [M+2H]²⁺: 1137.7.

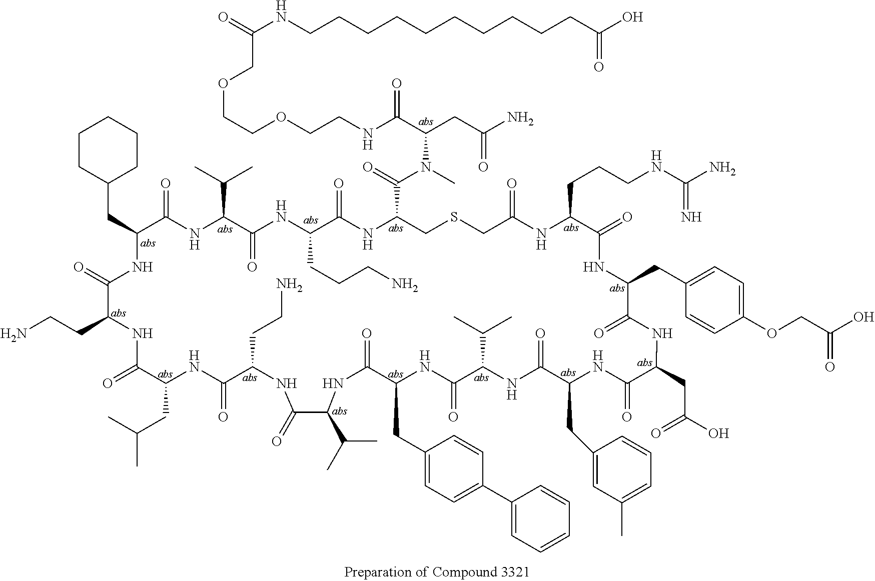 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N(C)[C@@H](CC(N)=O)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3321 was prepared on a 50 μmol scale. The yield of the product was 13.1 mg, and its estimated purity by LCMS analysis was 97.4%. Analysis condition A: Retention time=1.62 min; ESI-MS(+) m/z [M+2H]²⁺: 1187.

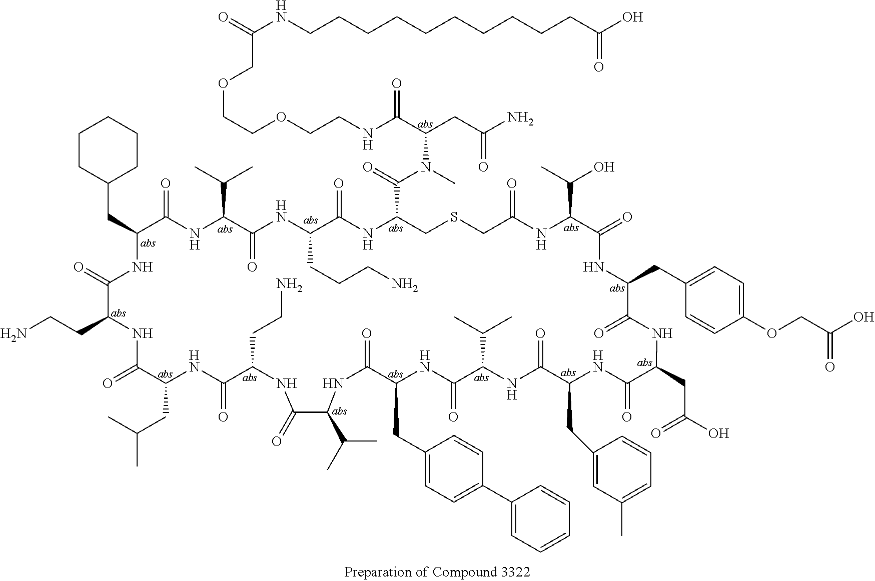 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)[C@@H](CC(N)=O)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3322 was prepared on a 50 μmol scale. The yield of the product was 3.9 mg, and its estimated purity by LCMS analysis was 98.1%. Analysis condition B: Retention time=1.6 min; ESI-MS(+) m/z [M+3H]³⁺: 773.6.

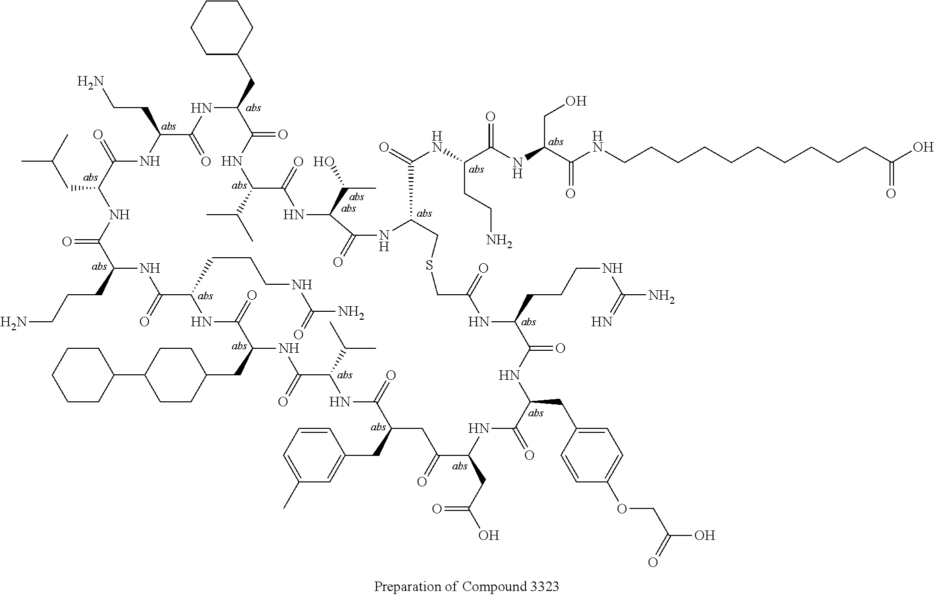 [SMILES: Cc1cccc(C[C@@H]2CC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)N[C@@H](CO)C(=O)NCCCCCCCCCCC(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](CC3CCC(C4CCCCC4)CC3)NC(=O)[C@H](C(C)C)NC2=O)c1]

Compound 3323 was prepared on a 50 μmol scale. The yield of the product was 13.3 mg, and its estimated purity by LCMS analysis was 98.3%. Analysis condition A: Retention time=1.59 min; ESI-MS(+) m/z [M+3H]³⁺: 783.

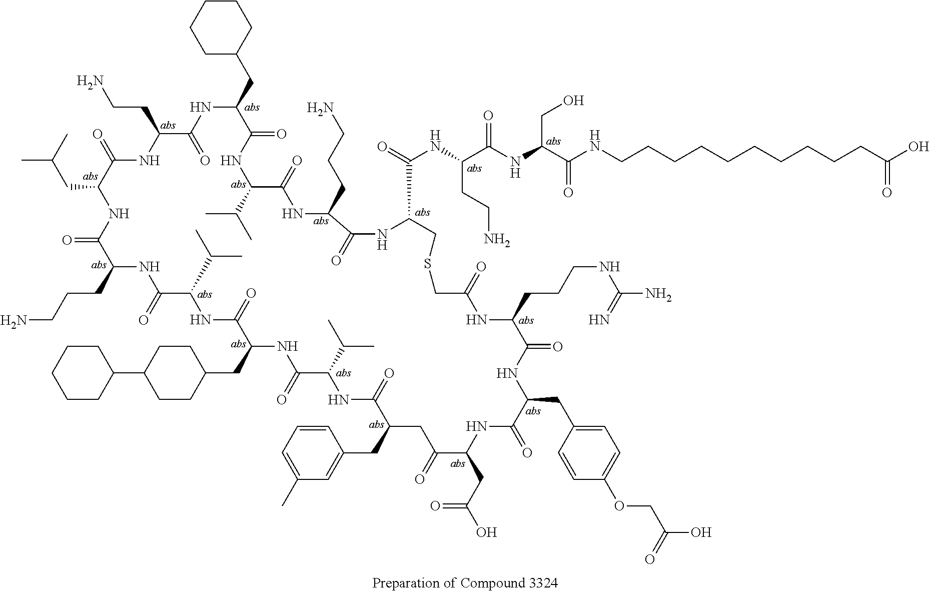 [SMILES: Cc1cccc(C[C@@H]2CC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)N[C@@H](CO)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](CC3CCC(C4CCCCC4)CC3)NC(=O)[C@H](C(C)C)NC2=O)c1]

Compound 3324 was prepared on a 50 μmol scale. The yield of the product was 11.4 mg, and its estimated purity by LCMS analysis was 100%. Analysis condition A: Retention time=1.52 min; ESI-MS(+) m/z [M+2H]²⁺: 1151.2.

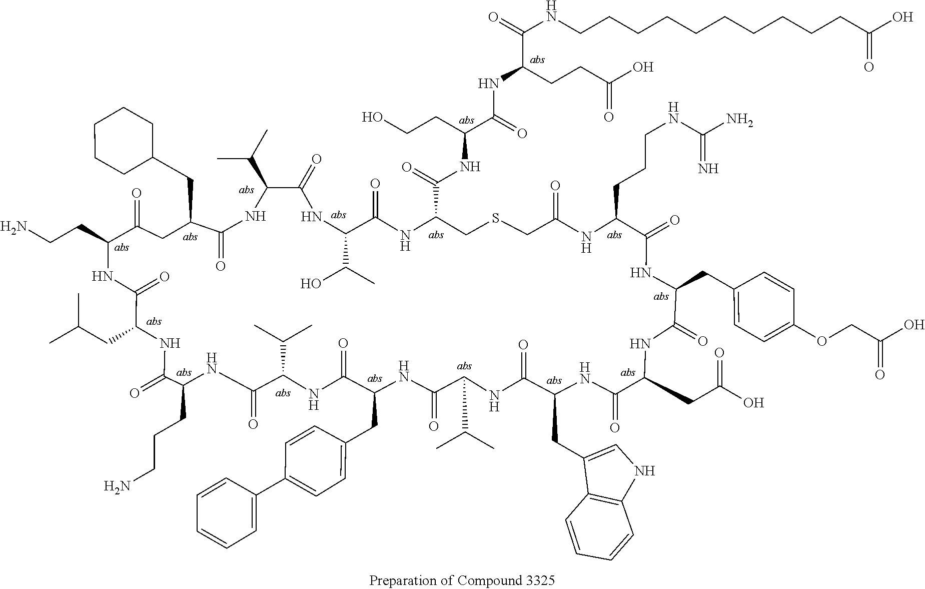 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCO)C(=O)N[C@H](CCC(=O)O)C(=O)NCCCCCCCCCCC(=O)O)NC(=O)[C@H](C(C)O)NC(=O)[C@H](C(C)C)NC(=O)[C@H](CC2CCCCC2)CC(=O)[C@H](CCN)NC1=O]

Compound 3325 was prepared on a 50 μmol scale. The yield of the product was 7.9 mg, and its estimated purity by LCMS analysis was 96.6%. Analysis condition A: Retention time=1.63 min; ESI-MS(+) m/z [M+3H]³⁺: 786.3.

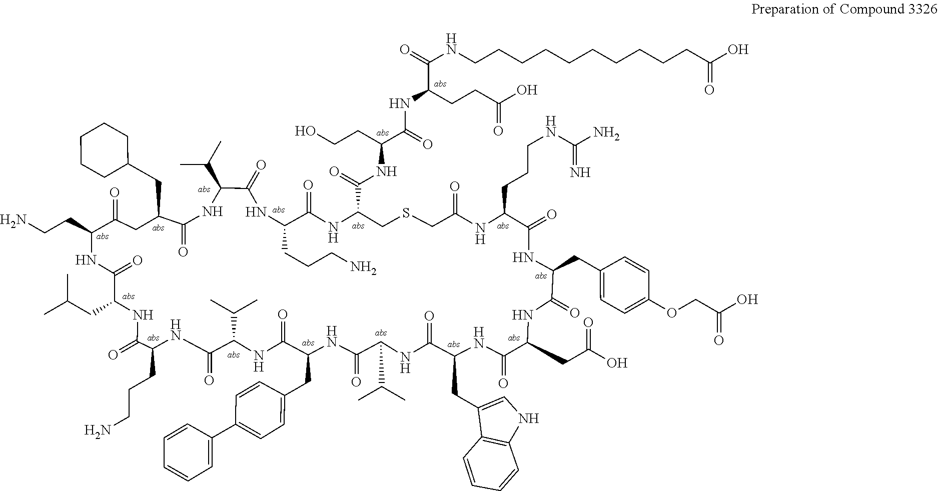 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCO)C(=O)N[C@H](CCC(=O)O)C(=O)NCCCCCCCCCCC(=O)O)NC(=O)[C@H](CCCN)NC(=O)[C@H](C(C)C)NC(=O)[C@H](CC2CCCCC2)CC(=O)[C@H](CCN)NC1=O]

Compound 3326 was prepared on a 50 μmol scale. The yield of the product was 15.1 mg, and its estimated purity by LCMS analysis was 95.4%. Analysis condition A: Retention time-1.56 min; ESI-MS(+) m/z [M+2H]²⁺: 1184.9.

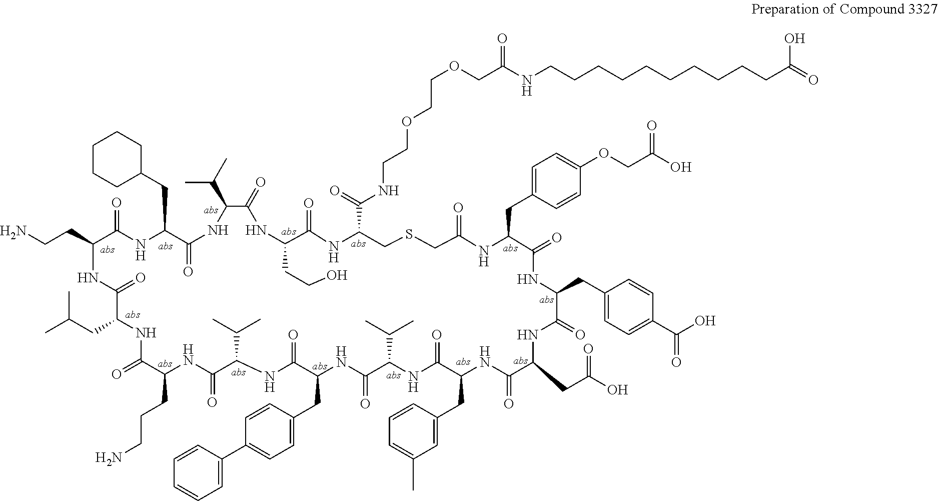 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)NCCOCCOCC(=O)NCCCCCCCCCCC(=O)O)NC(=O)C[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3327 was prepared on a 50 μmol scale. The yield of the product was 1.8 mg, and its estimated purity by LCMS analysis was 96.4%. Analysis condition B: Retention time=1.88 min; ESI-MS(+) m/z [M+2H]²⁺: 1141.1.

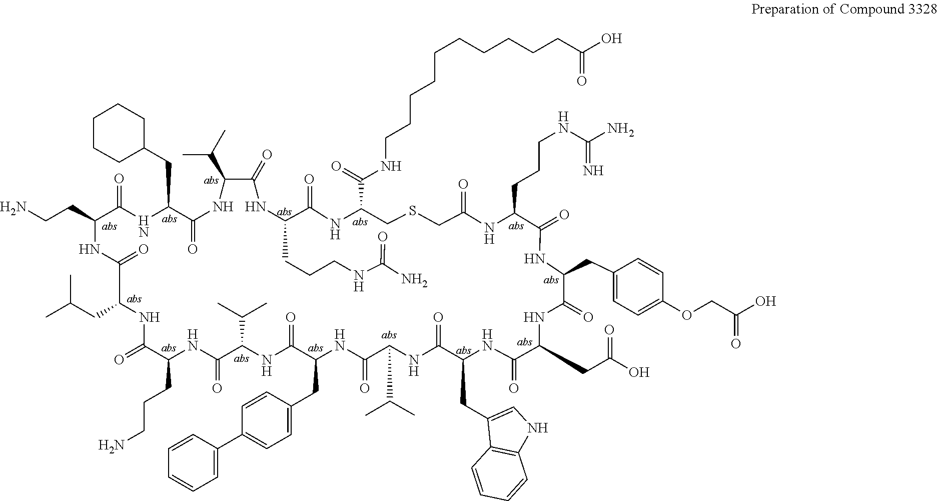 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCCCCCCCCCC(=O)O)NC(=O)C[C@H](CCCNC(N)=O)NC(=O)[C@H](C(C)C)NC(=O)[C@H](CC2CCCCC2)NC(=O)[C@H](CCN)NC1=O]

Compound 3328 was prepared on a 50 μmol scale. The yield of the product was 34.1 mg, and its estimated purity by LCMS analysis was 97.1%. Analysis condition A: Retention time=1.67 min; ESI-MS(+) m/z [M+2H]²⁺: 1084.2.

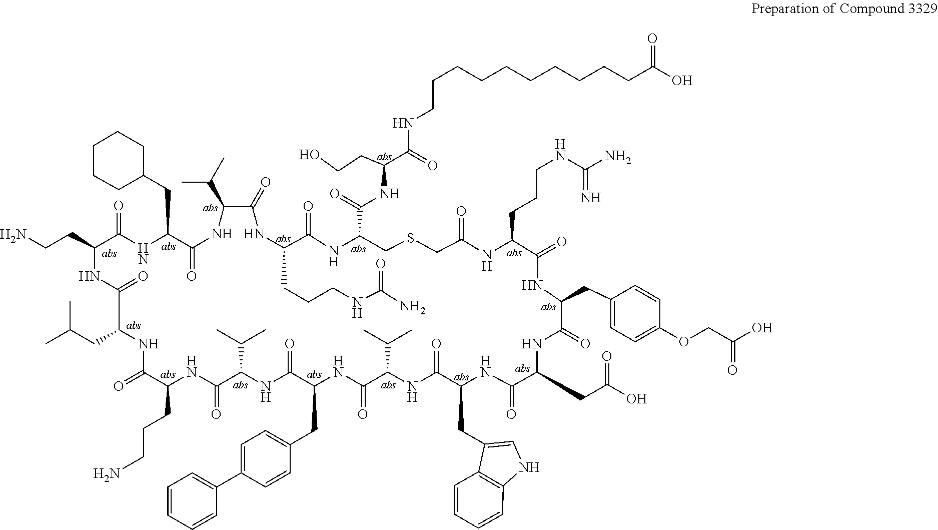 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCO)C(=O)NCCCCCCCCCCC(=O)O)NC(=O)C[C@H](CCCNC(N)=O)NC(=O)[C@H](C(C)C)NC(=O)[C@H](CC2CCCCC2)NC(=O)[C@H](CCN)NC1=O]

Compound 3329 was prepared on a 50 μmol scale. The yield of the product was 9.9 mg, and its estimated purity by LCMS analysis was 95.9%. Analysis condition A: Retention time=1.58 min; ESI-MS(+) m/z [M+3H]³⁺: 747.1.

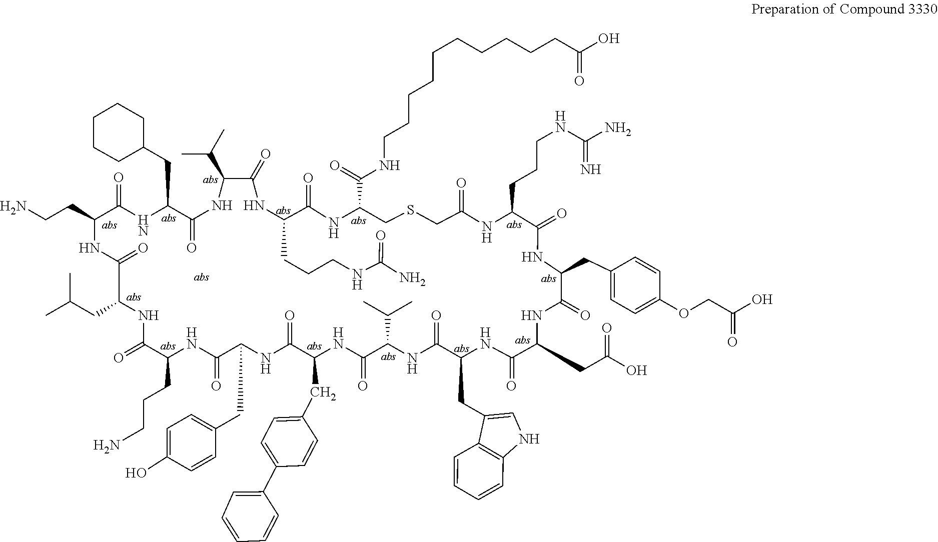 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCCCCCCCCCC(=O)O)NC(=O)C[C@H](CCCNC(N)=O)NC(=O)[C@H](C(C)C)NC(=O)[C@H](CC2CCCCC2)NC(=O)[C@H](CCN)NC1=O]

Compound 3330 was prepared on a 50 μmol scale. The yield of the product was 22 mg, and its estimated purity by LCMS analysis was 98.4%. Analysis condition A: Retention time=1.76 min; ESI-MS(+) m/z [M+2H]²⁺: 1116.3.

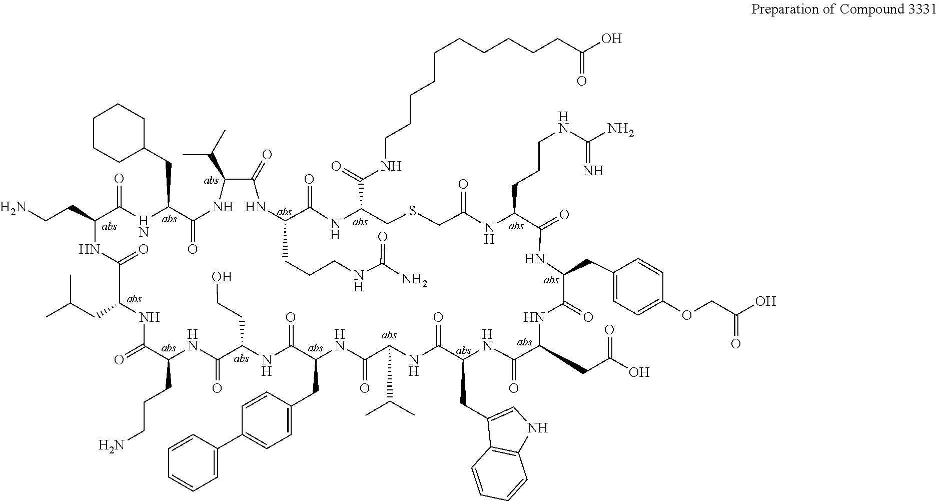 [SMILES: CC(C)C[C@H]1NC(=O)[C@H](CCCN)NC(=O)[C@H](CCO)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCCCCCCCCCC(=O)O)NC(=O)C[C@H](CCCNC(N)=O)NC(=O)[C@H](C(C)C)NC(=O)[C@H](CC2CCCCC2)NC(=O)[C@H](CCN)NC1=O]

Compound 3331 was prepared on a 50 μmol scale. The yield of the product was 22.3 mg, and its estimated purity by LCMS analysis was 95.4%. Analysis condition B: Retention time=1.65 min; ESI-MS(+) m/z [M+2H]²⁺: 1085.1.

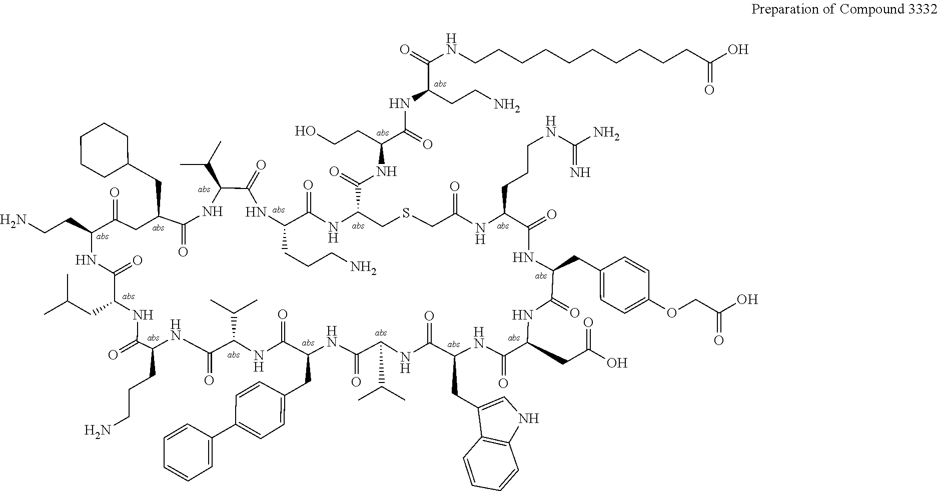 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCO)C(=O)N[C@H](CCN)C(=O)NCCCCCCCCCCC(=O)O)NC(=O)[C@H](CCCN)NC(=O)[C@H](C(C)C)NC(=O)[C@H](CC2CCCCC2)CC(=O)[C@H](CCN)NC1=O]

Compound 3332 was prepared on a 50 μmol scale. The yield of the product was 5.5 mg, and its estimated purity by LCMS analysis was 100%. Analysis condition A: Retention time=1.5, 1.62 min; ESI-MS(+) m/z [M+3H]³⁺: 776.03, 1163.94.

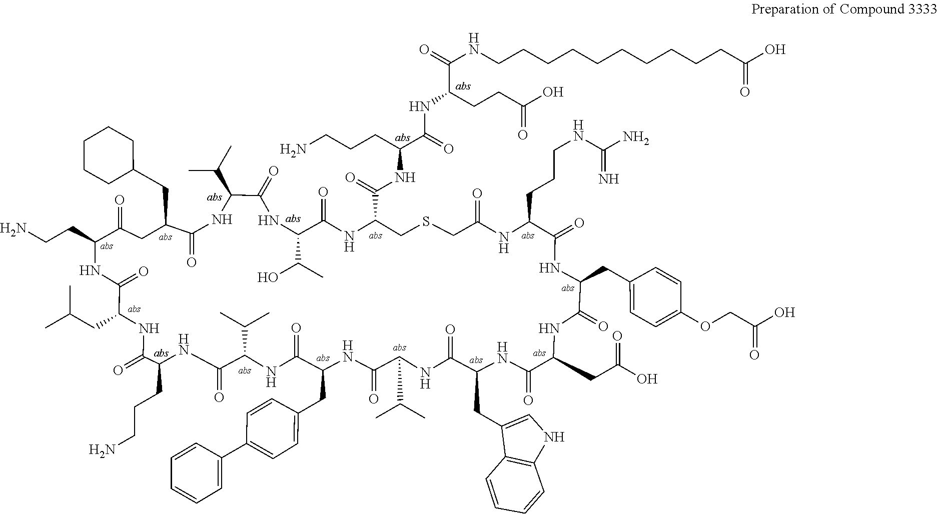 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)N[C@@H](CCC(=O)O)C(=O)NCCCCCCCCCCC(=O)O)NC(=O)[C@H](C(C)O)NC(=O)[C@H](C(C)C)NC(=O)[C@H](CC2CCCCC2)CC(=O)[C@H](CCN)NC1=O]

Compound 3333 was prepared on a 50 μmol scale. The yield of the product was 23.7 mg, and its estimated purity by LCMS analysis was 96%. Analysis condition A: Retention time=1.56 min; ESI-MS(+) m/z [M+2H]²⁺: 1185.1.

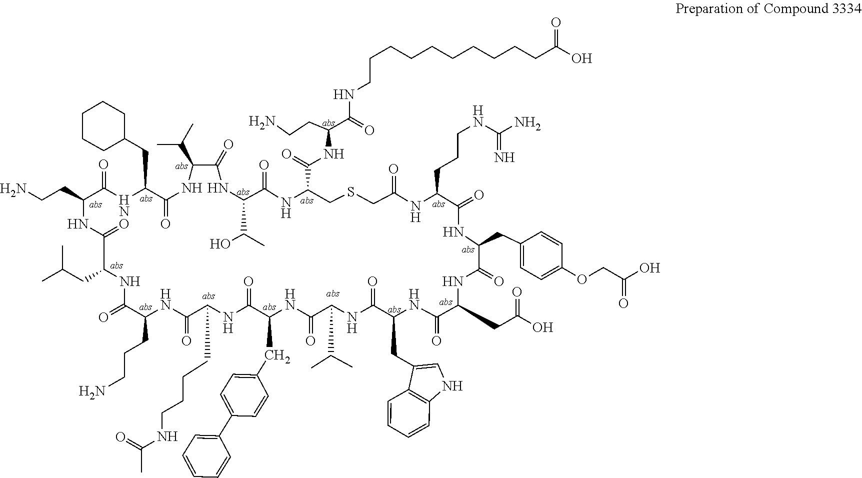 [SMILES: CC(=O)NCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)C[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3334 was prepared on a 50 μmol scale. The yield of the product was 29.4 mg, and its estimated purity by LCMS analysis was 95.9%. Analysis condition A: Retention time=1.52 min; ESI-MS(+) m/z [M+2H]²⁺: 1128.1.

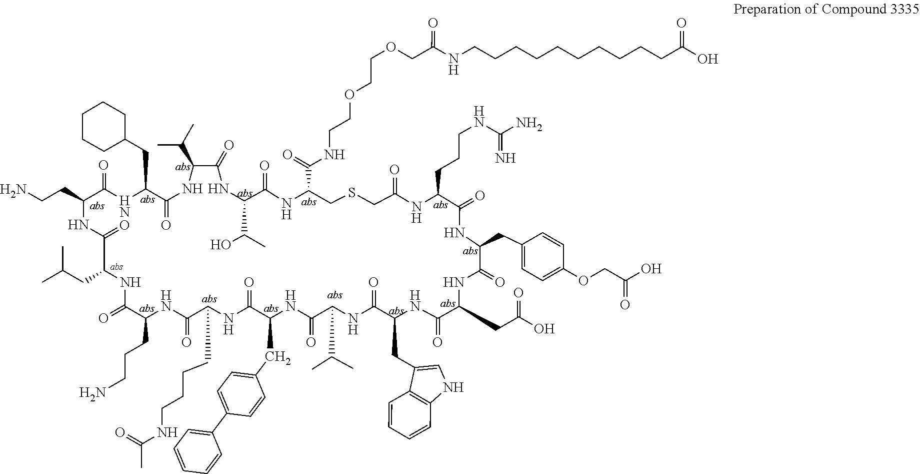 [SMILES: CC(=O)NCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCOCCOCC(=O)NCCCCCCCCCCC(=O)O)NC(=O)C[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3335 was prepared on a 50 μmol scale. The yield of the product was 48.2 mg, and its estimated purity by LCMS analysis was 100%. Analysis condition A: Retention time=1.63 min; ESI-MS(+) m/z [M+2H]²⁺: 1171.1.

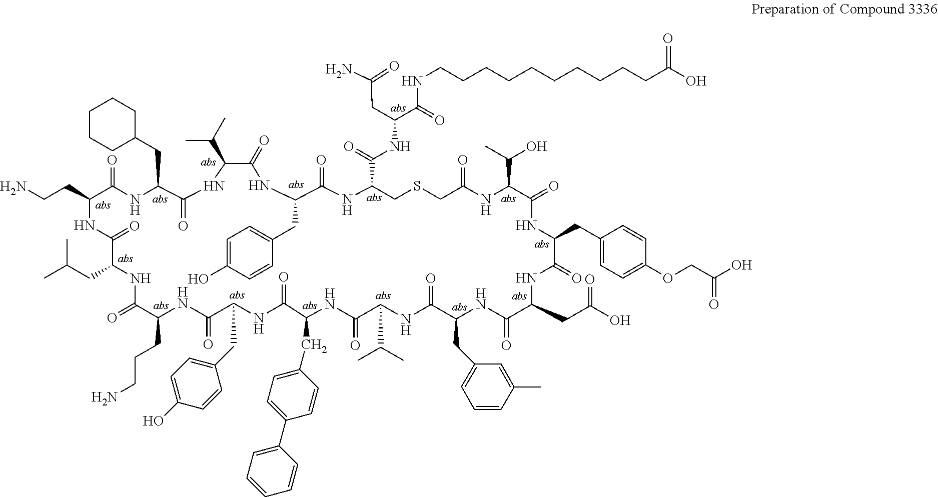 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](Cc3ccc(O)cc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3336 was prepared on a 50 μmol scale. The yield of the product was 53.5 mg, and its estimated purity by LCMS analysis was 98.3%. Analysis condition B: Retention time=1.61 min; ESI-MS(+) m/z [M+2H]²⁺: 1125.8.

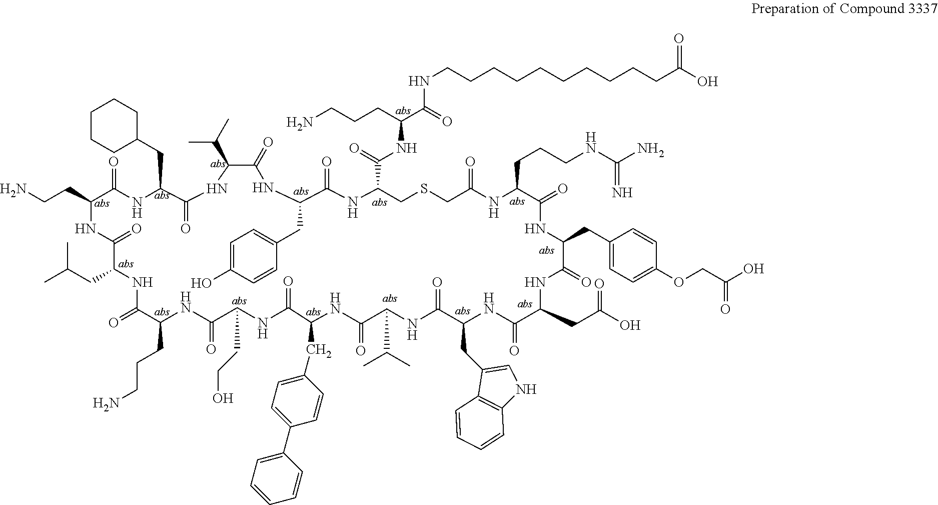 [SMILES: CC(C)C[C@H]1NC(=O)[C@H](CCCN)NC(=O)[C@H](CCO)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCCCCCCCCCC(=O)O)NC(=O)[C@H](Cc2ccc(O)cc2)NC(=O)[C@H](C(C)C)NC(=O)[C@H](CC2CCCCC2)NC(=O)[C@H](CCN)NC1=O]

Compound 3337 was prepared on a 50 μmol scale. The yield of the product was 21.8 mg, and its estimated purity by LCMS analysis was 95.3%. Analysis condition B: Retention time=1.49 min; ESI-MS(+) m/z [M+3H]³⁺: 748.3.

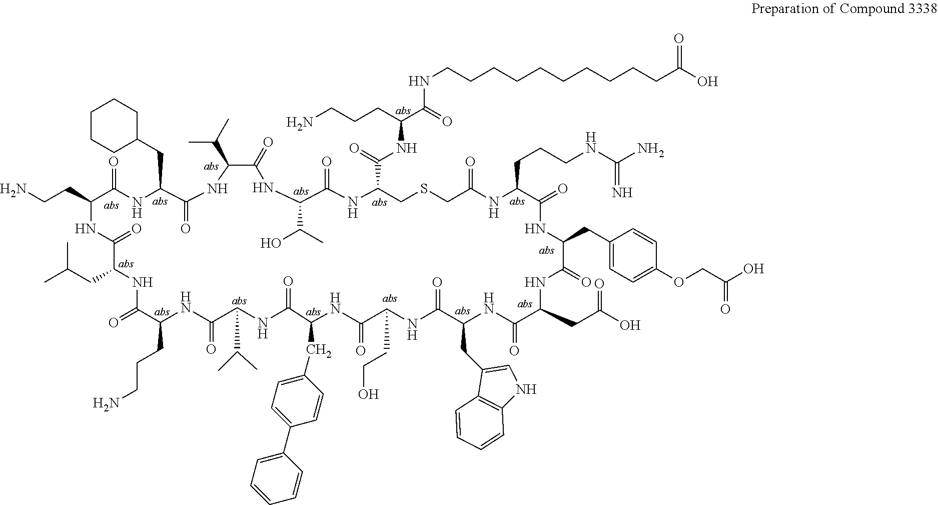 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](CCO)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3338 was prepared on a 50 μmol scale. The yield of the product was 12.4 mg, and its estimated purity by LCMS analysis was 95.5%. Analysis condition A: Retention time=1.57 min; ESI-MS(+) m/z [M+3H]³⁺: 748.2.

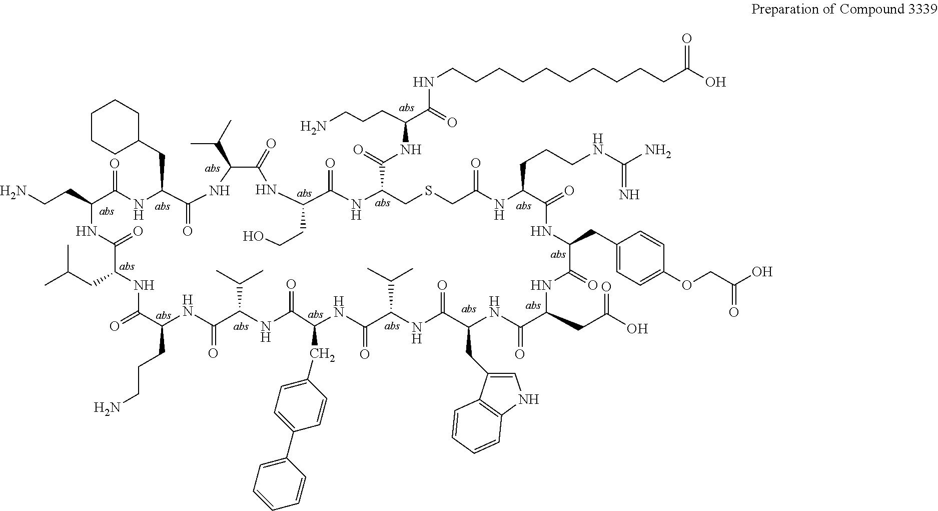 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC1=O]

Compound 3339 was prepared on a 50 μmol scale. The yield of the product was 4.7 mg, and its estimated purity by LCMS analysis was 95%. Analysis condition A: Retention time=1.62 min; ESI-MS(+) m/z [M+3H]³⁺: 747.1.

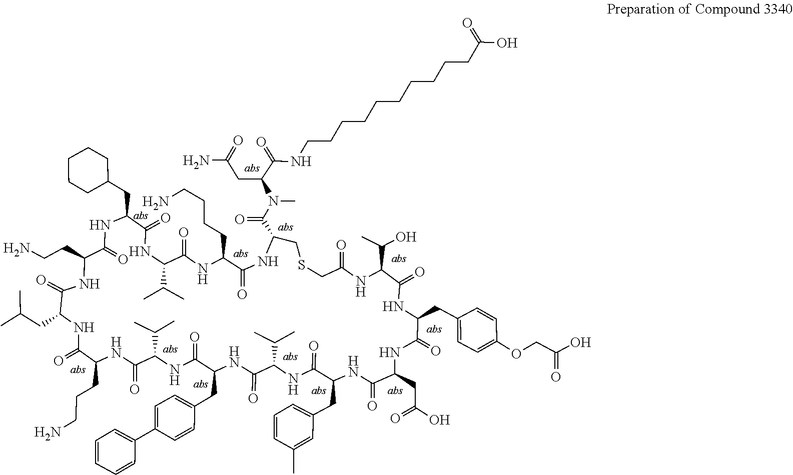

Compound 3340 was prepared on a 50 μmol scale. The yield of the product was 4 mg, and its estimated purity by LCMS analysis was 97%. Analysis condition A: Retention time=1.66 min; ESI-MS(+) m/z [M+2H]²⁺: 1101.1.

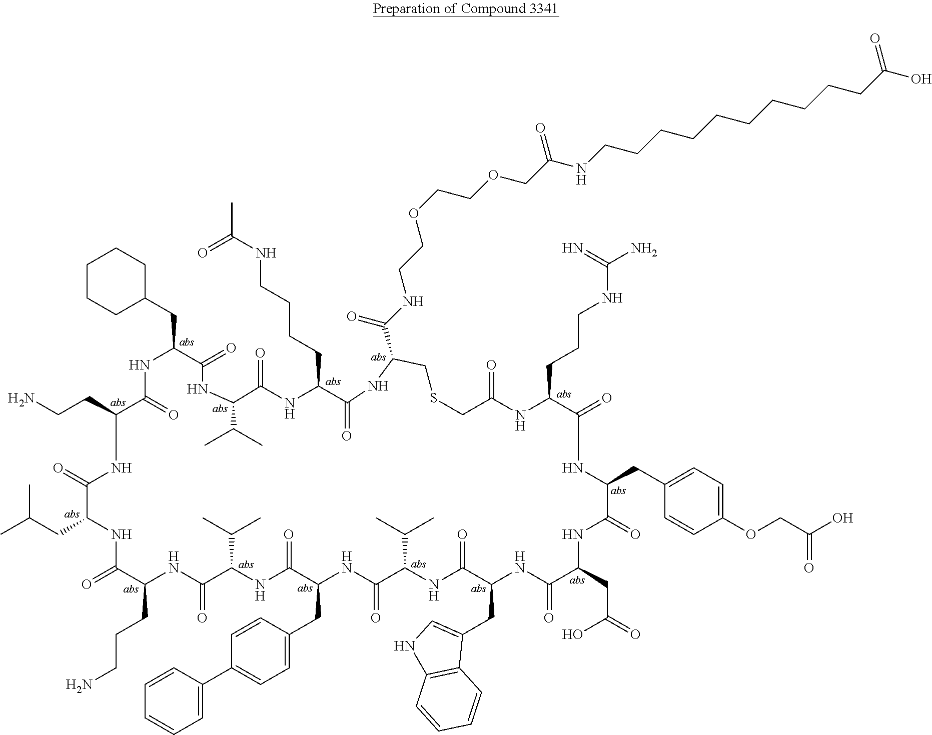 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCOCCOCC(=O)NCCCCCCCCCCC(=O)O)NC1=O]

Compound 3341 was prepared on a 50 μmol scale. The yield of the product was 10.7 mg, and its estimated purity by LCMS analysis was 96.4%. Analysis condition A: Retention time=1.61 min; ESI-MS(+) m/z [M+3H]³⁺: 781.1.

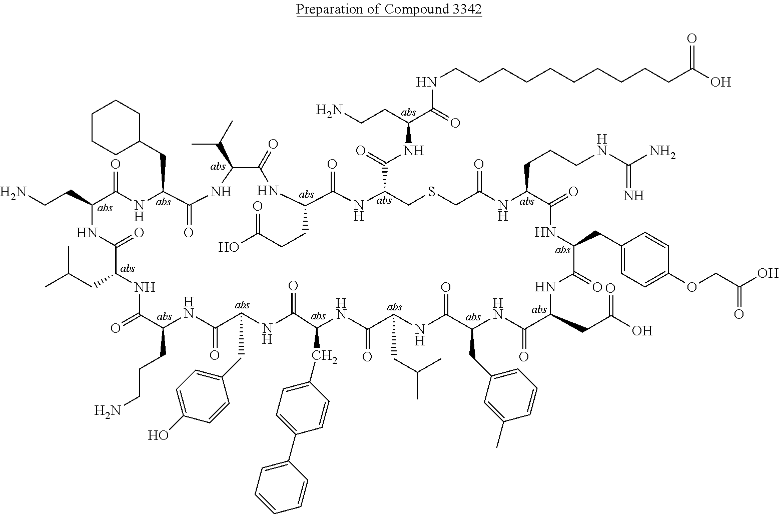 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CC(C)C)NC2=O)c1]

Compound 3342 was prepared on a 50 μmol scale. The yield of the product was 44.7 mg, and its estimated purity by LCMS analysis was 100%. Analysis condition A: Retention time=1.81 min; ESI-MS(+) m/z [M+2H]²⁺: 1154.2.

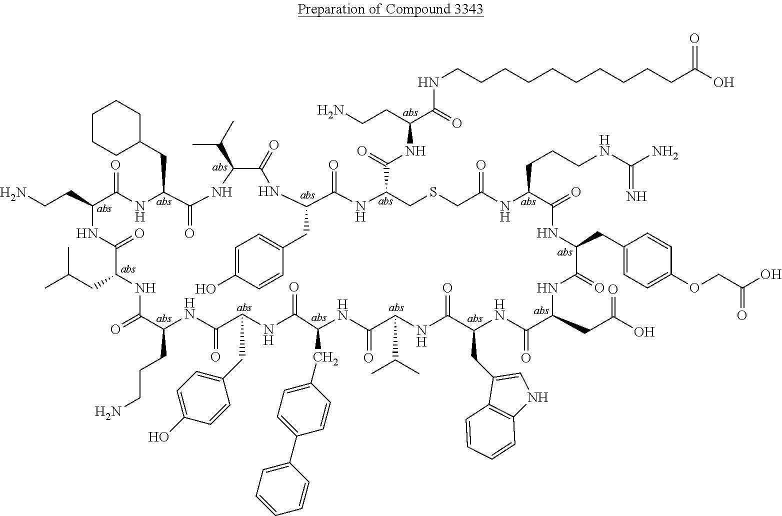 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](Cc2ccc(O)cc2)NC(=O)[C@H](C(C)C)NC(=O)[C@H](CC2CCCCC2)NC(=O)[C@H](CCN)NC1=O]

Compound 3343 was prepared on a 50 μmol scale. The yield of the product was 34.9 mg, and its estimated purity by LCMS analysis was 99%. Analysis condition A: Retention time=1.52 min; ESI-MS(+) m/z [M+3H]³⁺: 784.9.

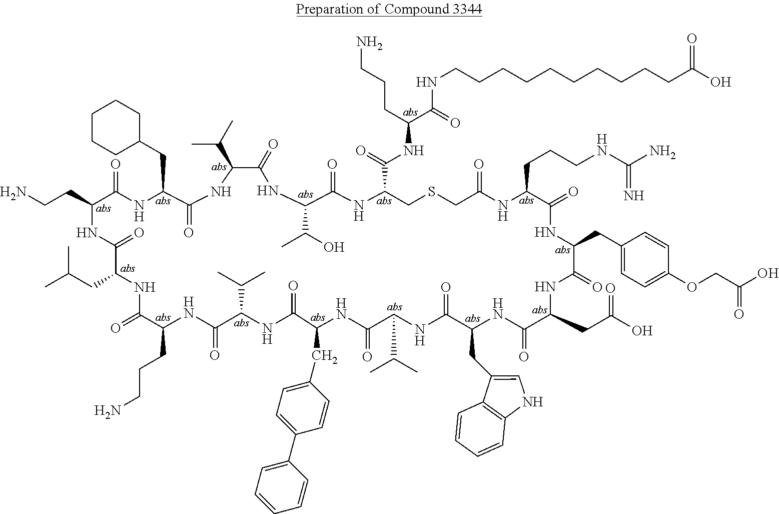 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3344 was prepared on a 50 μmol scale. The yield of the product was 35.4 mg, and its estimated purity by LCMS analysis was 95.5%. Analysis condition B: Retention time=1.5 min; ESI-MS(+) m/z [M+2H]²⁺: 1121.

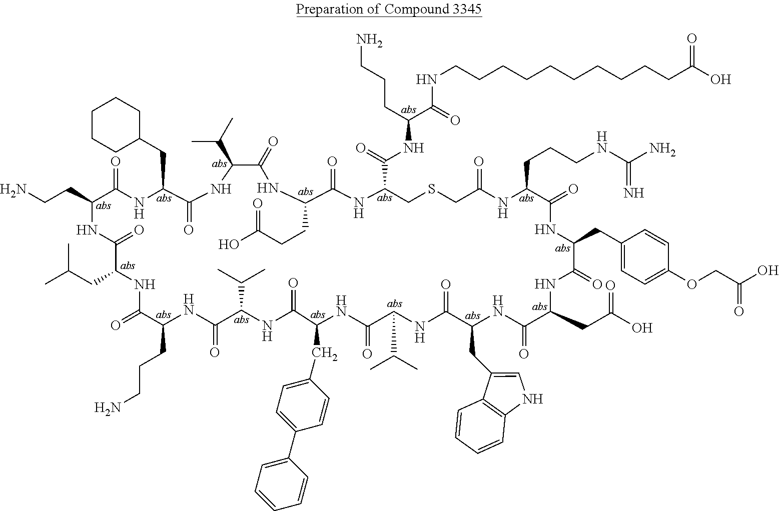 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3345 was prepared on a 50 μmol scale. The yield of the product was 7.4 mg, and its estimated purity by LCMS analysis was 90.2%. Analysis condition B: Retention time=1.5 min; ESI-MS(+) m/z [M+3H]³⁺: 756.6.

### Preparation of Compound 3346

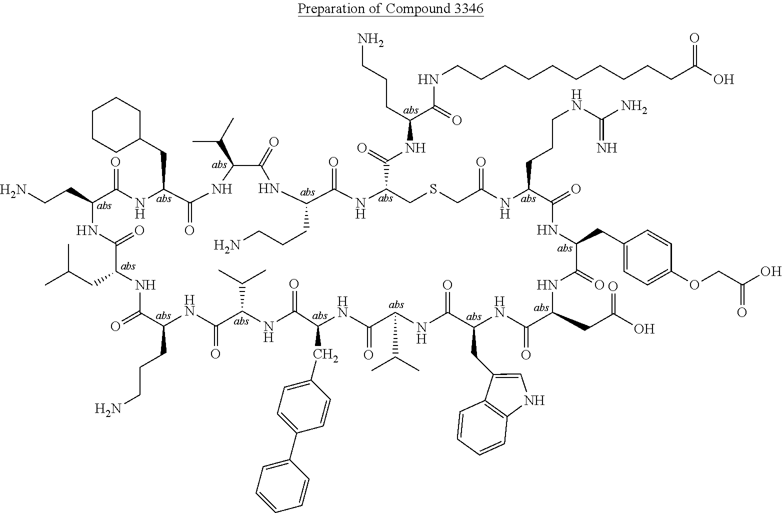 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCCN)C(=O)NCCCCCCCCCCC(=O)O)NC(=O)[C@H](CCCN)NC(=O)[C@H](C(C)C)NC(=O)[C@H](CC2CCCCC2)NC(=O)[C@H](CCN)NC1=O]

Compound 3346 was prepared on a 50 μmol scale. The yield of the product was 29.5 mg, and its estimated purity by LCMS analysis was 96.5%. Analysis condition B: Retention time=1.44 min; ESI-MS(+) m/z [M+2H]²⁺: 1128.3.

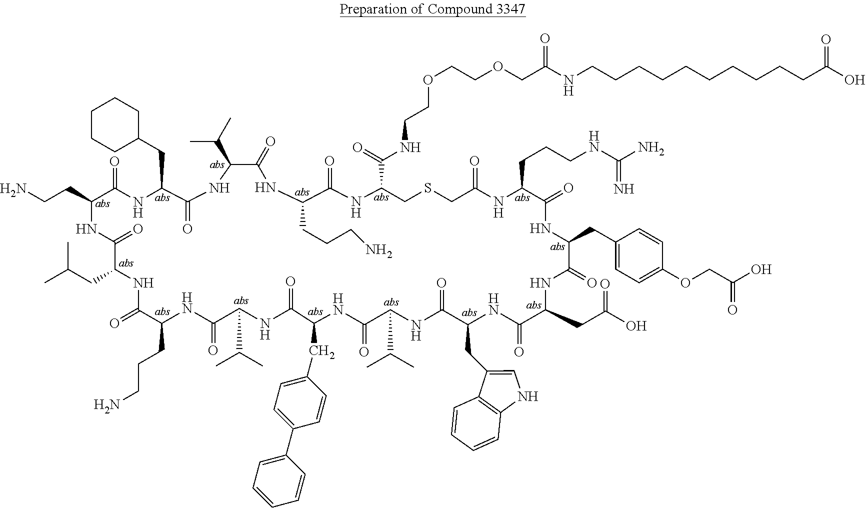

Compound 3347 was prepared on a 50 μmol scale. The yield of the product was 24.2 mg, and its estimated purity by LCMS analysis was 100%. Analysis condition A: Retention time=1.57 min; ESI-MS(+) m/z [M+3H]³⁺: 762.2.

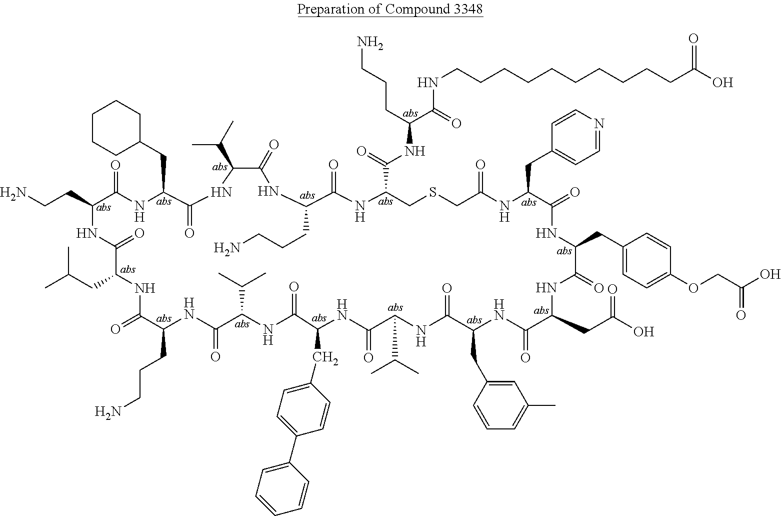 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3348 was prepared on a 50 μmol scale. The yield of the product was 10.6 mg, and its estimated purity by LCMS analysis was 98.2%. Analysis condition B: Retention time=1.47 min; ESI-MS(+) m/z [M+3H]³⁺: 741.

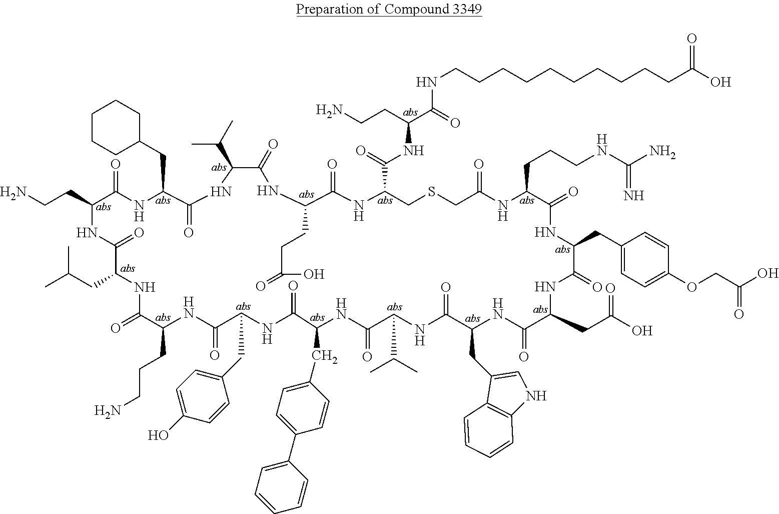 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3349 was prepared on a 50 μmol scale. The yield of the product was 11.4 mg, and its estimated purity by LCMS analysis was 97.8%. Analysis condition A: Retention time=1.46 min; ESI-MS(+) m/z [M+2H]²⁺: 1159.9.

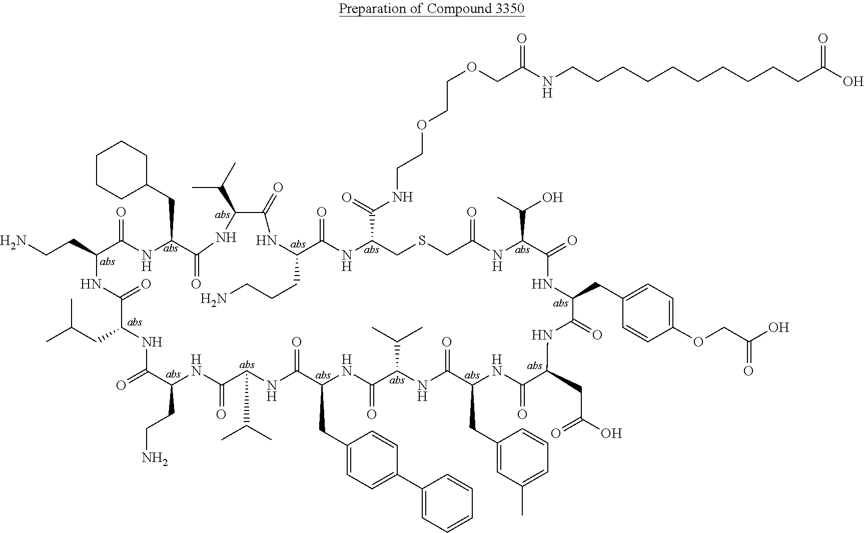 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3350 was prepared on a 50 μmol scale. The yield of the product was 15.3 mg, and its estimated purity by LCMS analysis was 95.2%. Analysis condition B: Retention time=1.69 min; ESI-MS(+) m/z [M+2H]²⁺: 1095.4.

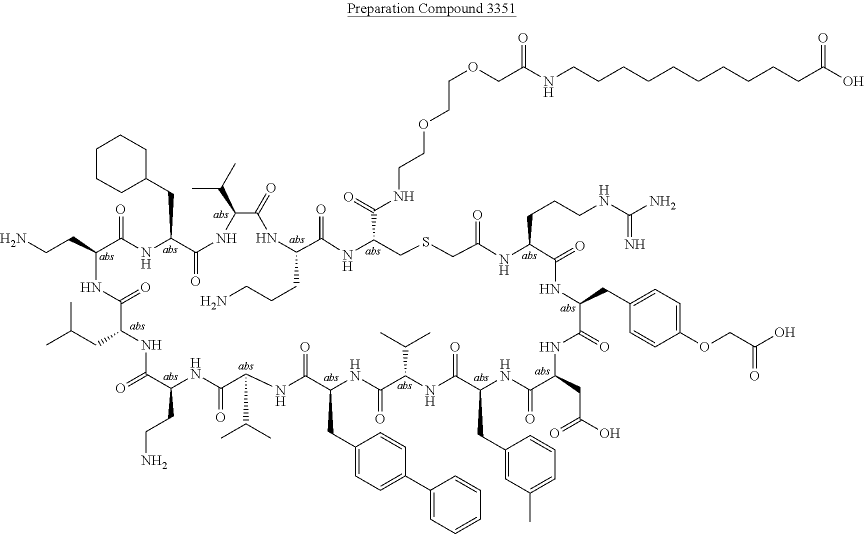 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3351 was prepared on a 50 μmol scale. The yield of the product was 7.2 mg, and its estimated purity by LCMS analysis was 100%. Analysis condition A: Retention time=1.65 min; ESI-MS(+) m/z [M+2H]²⁺: 1123.2.

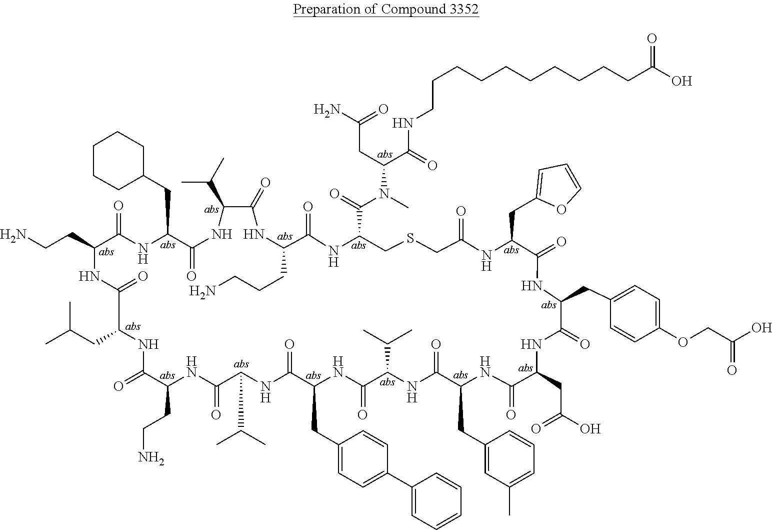 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccco3)NC(=O)CSC[C@@H](C(=O)N(C)[C@H](CC(N)=O)C(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3352 was prepared on a 50 μmol scale. The yield of the product was 23.2 mg, and its estimated purity by LCMS analysis was 100%. Analysis condition A: Retention time=1.76 min; ESI-MS(+) m/z [M+2H]²⁺: 1105.

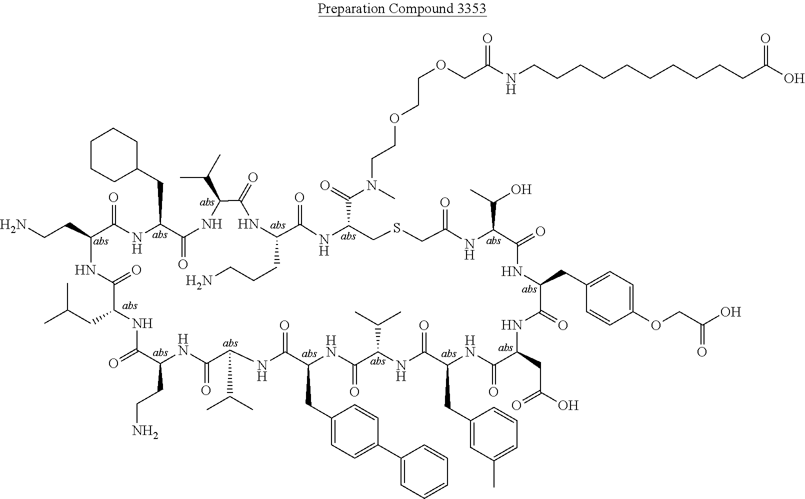 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3353 was prepared on a 50 μmol scale. The yield of the product was 4.1 mg, and its estimated purity by LCMS analysis was 95%. Analysis condition A: Retention time=1.59 min; ESI-MS(+) m/z [M+3H]³⁺: 735.4.

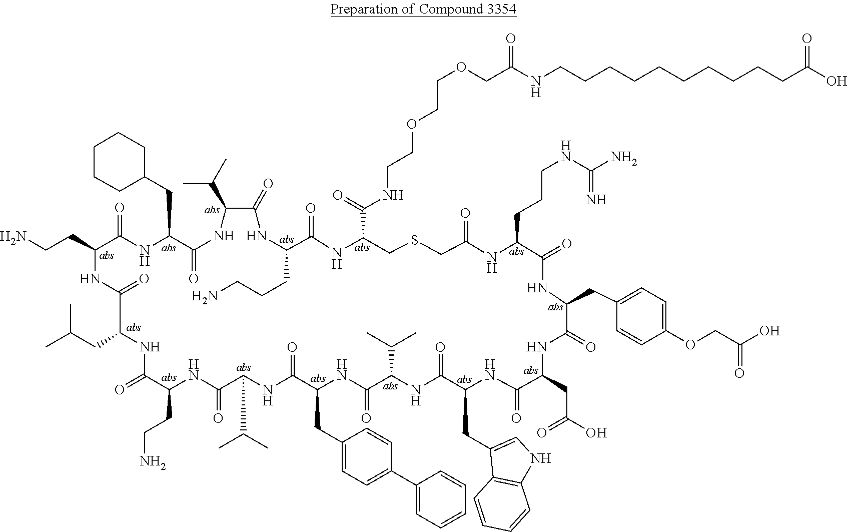 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCOCCOCC(=O)NCCCCCCCCCCC(=O)O)NC(=O)C[C@H](CCCN)NC(=O)[C@H](C(C)C)NC(=O)[C@H](CC2CCCCC2)NC(=O)[C@H](CCN)NC1=O]

Compound 3354 was prepared on a 50 μmol scale. The yield of the product was 12.3 mg, and its estimated purity by LCMS analysis was 92.3%. Analysis condition B: Retention time=1.55 min; ESI-MS(+) m/z [M+3H]³⁺: 758.2.

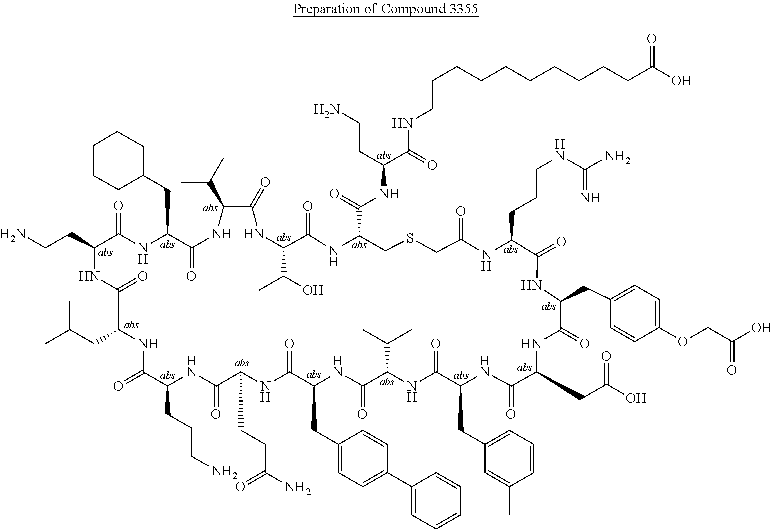 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)C[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 3355 was prepared on a 50 μmol scale. The yield of the product was 36 mg, and its estimated purity by LCMS analysis was 100%. Analysis condition A: Retention time=1.56 min; ESI-MS(+) m/z [M+3H]³⁺: 744.2.

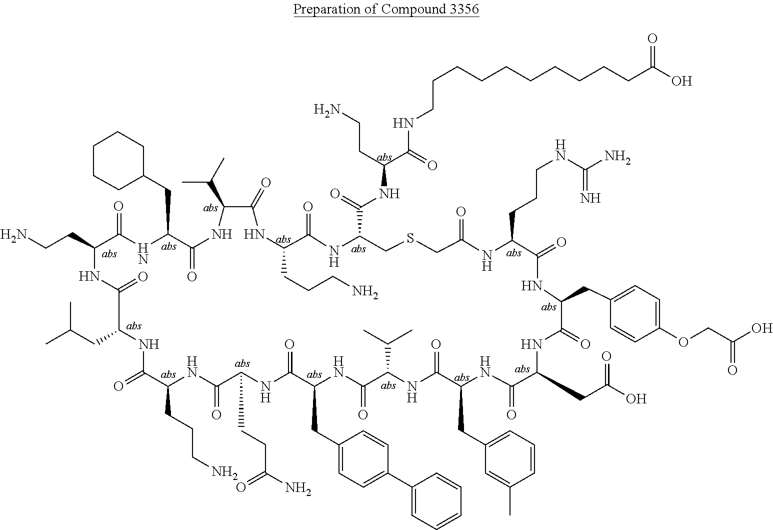 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 3356 was prepared on a 50 μmol scale. The yield of the product was 29.9 mg, and its estimated purity by LCMS analysis was 97.8%. Analysis condition B: Retention time=1.48 min; ESI-MS(+) m/z [M+2H]²⁺: 1121.8.

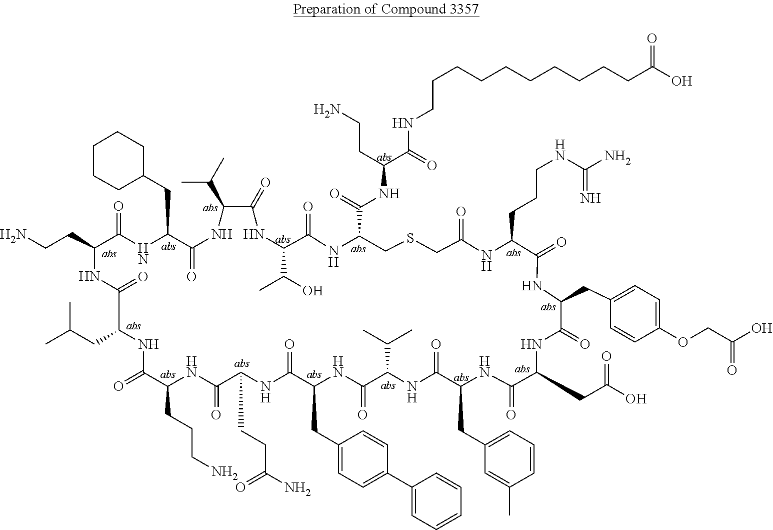 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)C[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 3357 was prepared on a 50 μmol scale. The yield of the product was 45.7 mg, and its estimated purity by LCMS analysis was 95.1%. Analysis condition B: Retention time=1.45 min; ESI-MS(+) m/z [M+3H]³⁺: 739.9.

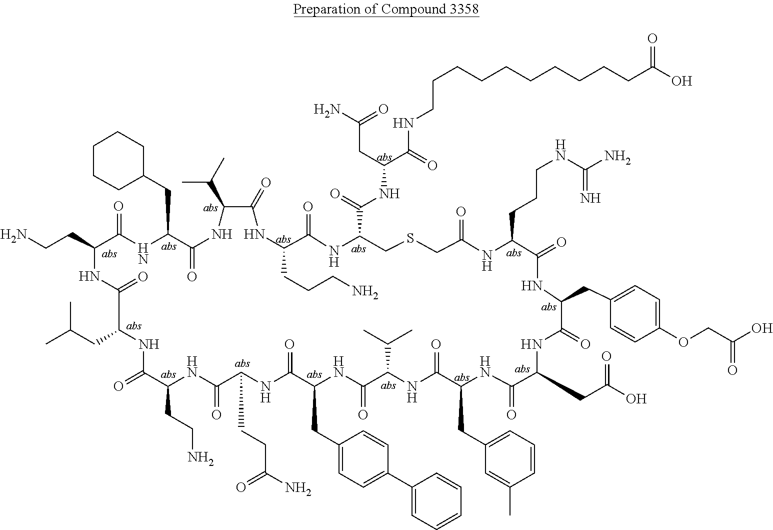 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCC(N)=O)NC(=O)[C@H](Cc3ccc(-c4ccccc4)cc3)NC(=O)[C@H](C(C)C)NC2=O)c1]

Compound 3358 was prepared on a 50 μmol scale. The yield of the product was 38.4 mg, and its estimated purity by LCMS analysis was 95.9%. Analysis condition B: Retention time=1.55 min; ESI-MS(+) m/z [M+2H]²⁺: 1136.9.

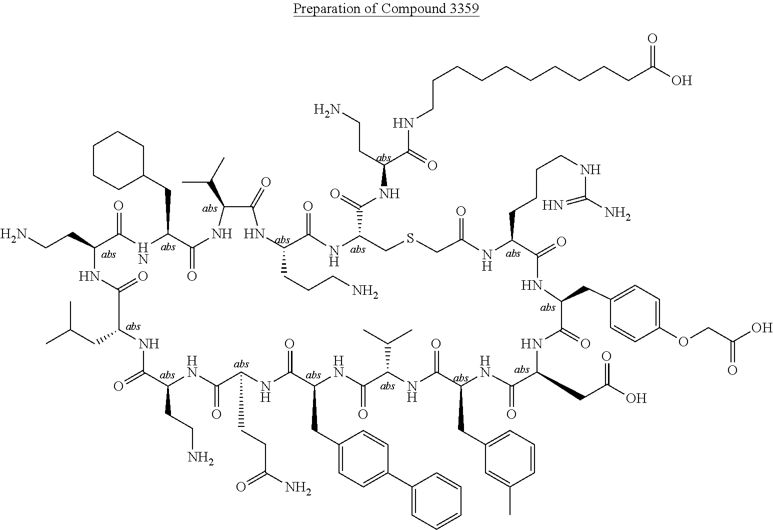 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)NC(=O)C[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](CCC(N)=O)NC(=O)[C@H](Cc3ccc(-c4ccccc4)cc3)NC(=O)[C@H](C(C)C)NC2=O)c1]

Compound 3359 was prepared on a 50 μmol scale. The yield of the product was 34.9 mg, and its estimated purity by LCMS analysis was 91.3%. Analysis condition B: Retention time=1.51 min; ESI-MS(+) m/z [M+2H]²⁺: 1122.1.

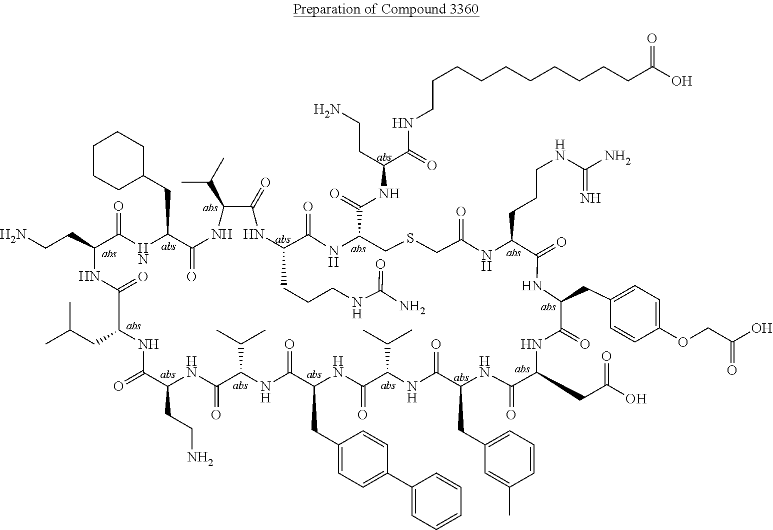 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)C[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3360 was prepared on a 50 μmol scale. The yield of the product was 14.4 mg, and its estimated purity by LCMS analysis was 100%. Analysis condition A: Retention time=1.53 min; ESI-MS(+) m/z [M+2H]²⁺: 1121.9.

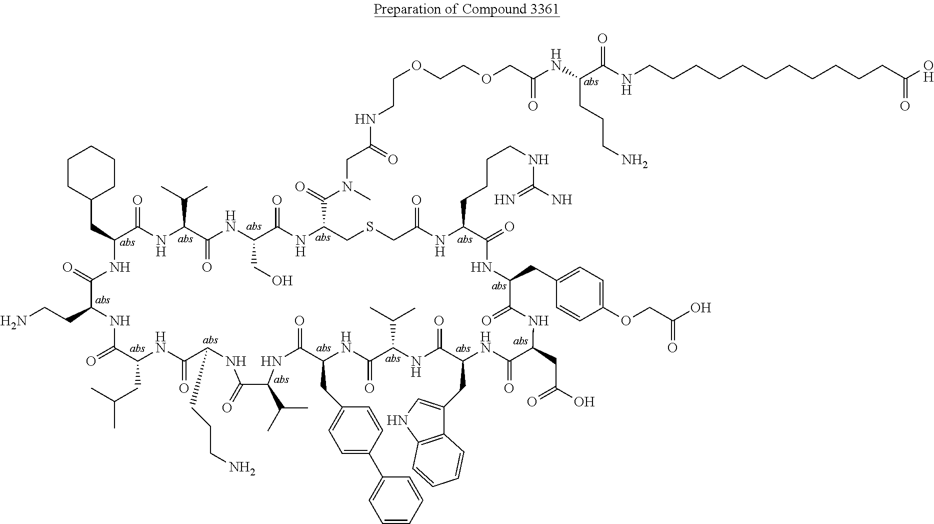 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N(C)CC(=O)NCCOCCOCC(=O)N[C@@H](CCCN)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3361 was prepared on a 50 μmol scale. The yield of the product was 20.5 mg, and its estimated purity by LCMS analysis was 100%. Analysis condition A: Retention time=1.52 min; ESI-MS(+) m/z [M+3H]³⁺: 824.1.

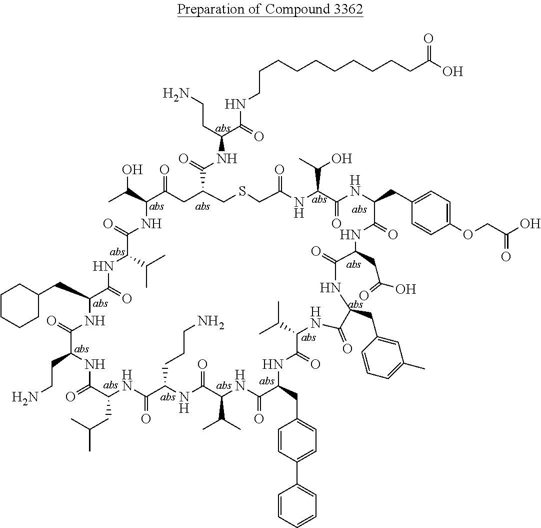 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)CC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3362 was prepared on a 50 μmol scale. The yield of the product was 10.9 mg, and its estimated purity by LCMS analysis was 95.5%. Analysis condition A: Retention time=1.76 min; ESI-MS(+) m/z [M+3H]³⁺: 716.

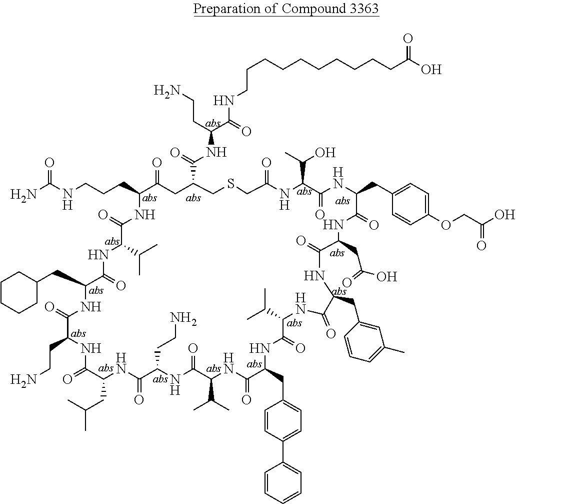 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)CC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3363 was prepared on a 50 μmol scale. The yield of the product was 6.2 mg, and its estimated purity by LCMS analysis was 100%. Analysis condition A: Retention time=1.65 min; ESI-MS(+) m/z [M+3H]³⁺: 730.

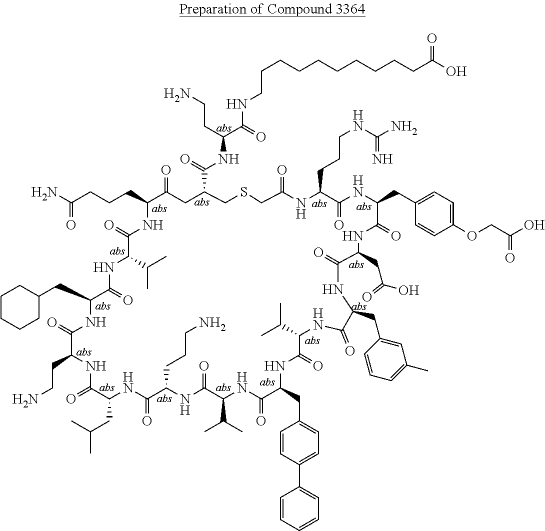 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)CC(=O)[C@H](CCCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3364 was prepared on a 50 μmol scale. The yield of the product was 1.2 mg, and its estimated purity by LCMS analysis was 91.6%. Analysis condition A: Retention time=1.57 min; ESI-MS(+) m/z [M+3H]³⁺: 748.

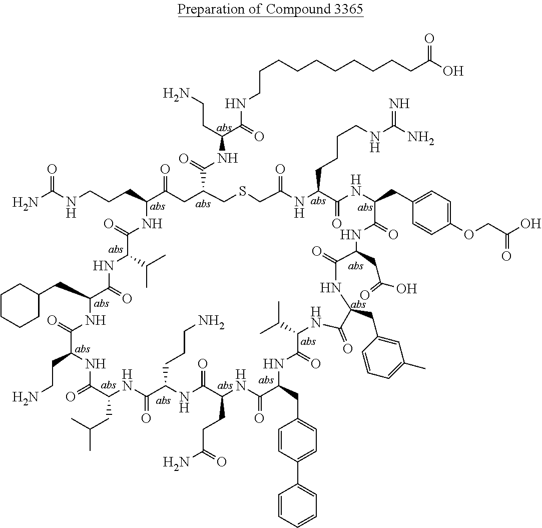 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)CC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 3365 was prepared on a 50 μmol scale. The yield of the product was 15.6 mg, and its estimated purity by LCMS analysis was 100%. Analysis condition A: Retention time=1.48 min; ESI-MS(+) m/z [M+3H]³⁺: 767.6.

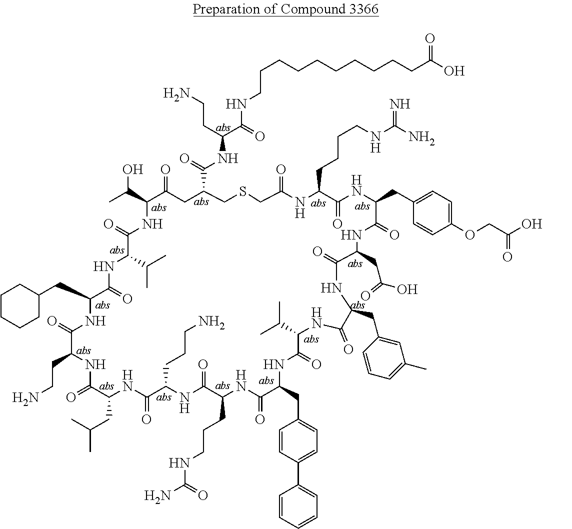 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)CC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3366 was prepared on a 50 μmol scale. The yield of the product was 11.1 mg, and its estimated purity by LCMS analysis was 94.9%. Analysis condition B: Retention time=1.53 min; ESI-MS(+) m/z [M+2H]²⁺: 1130.1.

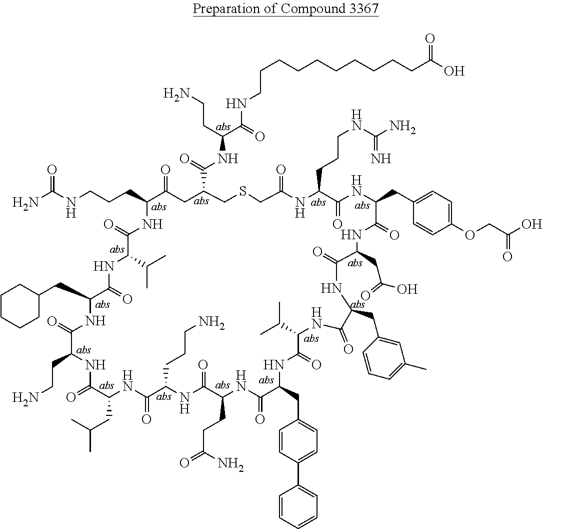 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)CC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 3367 was prepared on a 50 μmol scale. The yield of the product was 75 mg, and its estimated purity by LCMS analysis was 97.5%. Analysis condition B: Retention time=1.62 min; ESI-MS(+) m/z [M+3H]³⁺: 758.3.

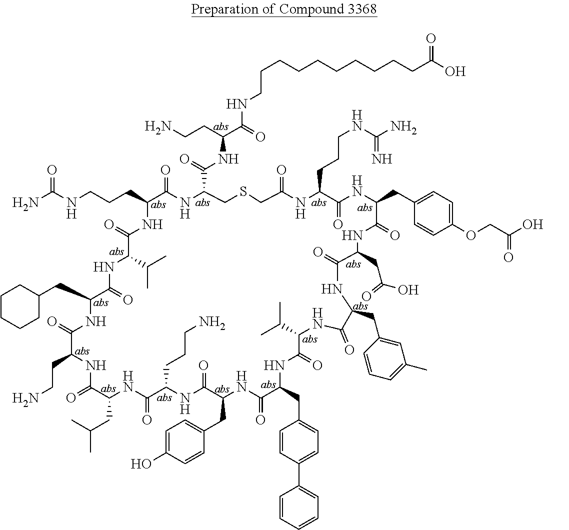 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3368 was prepared on a 50 μmol scale. The yield of the product was 14.6 mg, and its estimated purity by LCMS analysis was 96.2%. Analysis condition A: Retention time=1.7 min; ESI-MS(+) m/z [M+3H]³⁺: 793.1.

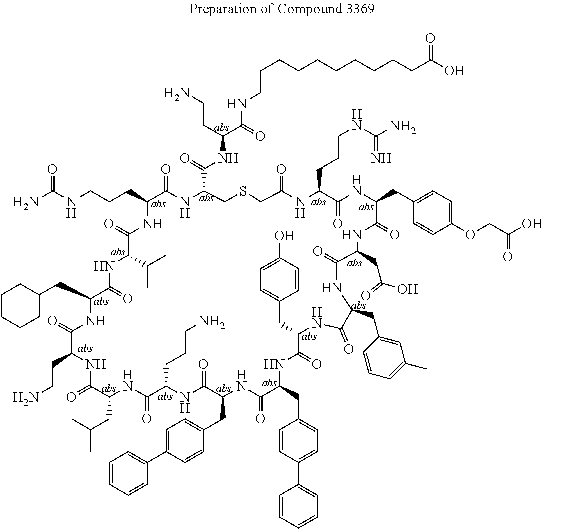 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(-c4ccccc4)cc3)NC(=O)[C@H](Cc3ccc(-c4ccccc4)cc3)NC(=O)[C@H](Cc3ccc(O)cc3)NC2=O)c1]

Compound 3369 was prepared on a 50 μmol scale. The yield of the product was 18.3 mg, and its estimated purity by LCMS analysis was 100%. Analysis condition A: Retention time=1.84, 1.89 min; ESI-MS(+) m/z [M+2H]²⁺: 1230.99, 1230.99.

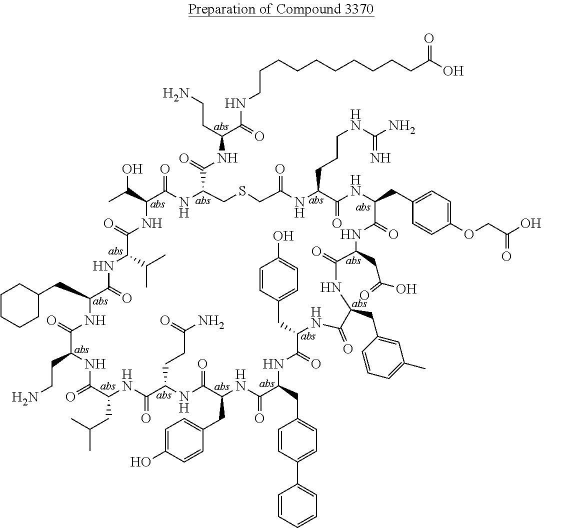 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 3370 was prepared on a 50 μmol scale. The yield of the product was 20.1 mg, and its estimated purity by LCMS analysis was 100%. Analysis condition A: Retention time=1.55 min; ESI-MS(+) m/z [M+2H]²⁺: 1172.3.

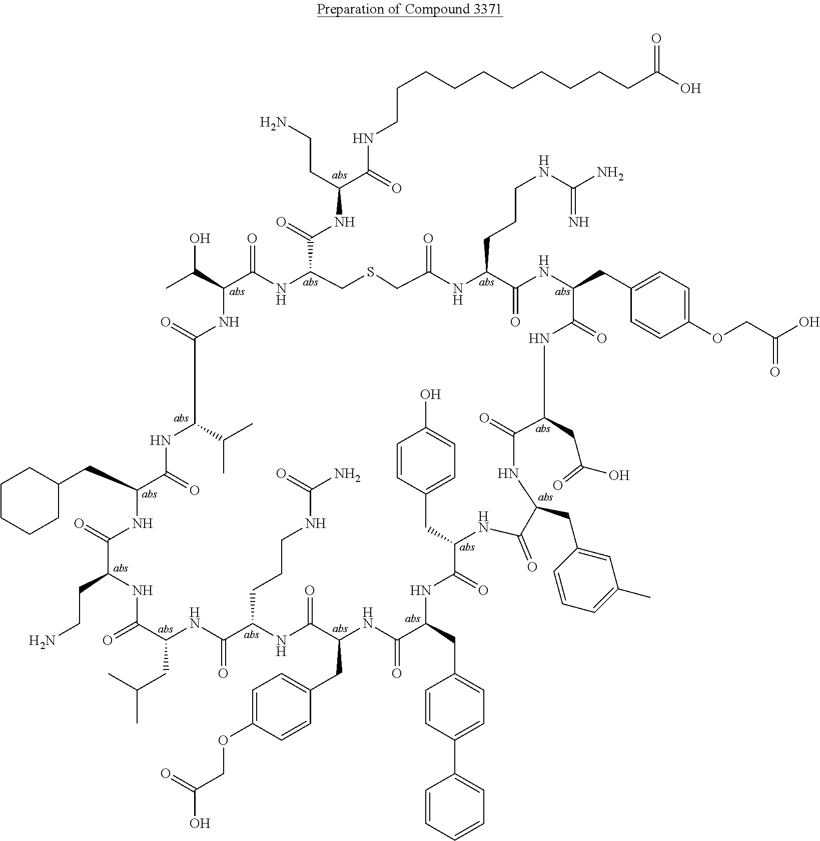 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 3371 was prepared on a 50 μmol scale. The yield of the product was 8.4 mg, and its estimated purity by LCMS analysis was 97.8%. Analysis condition A: Retention time=1.46 min; ESI-MS(+) m/z [M+2H]²⁺: 1215.3.

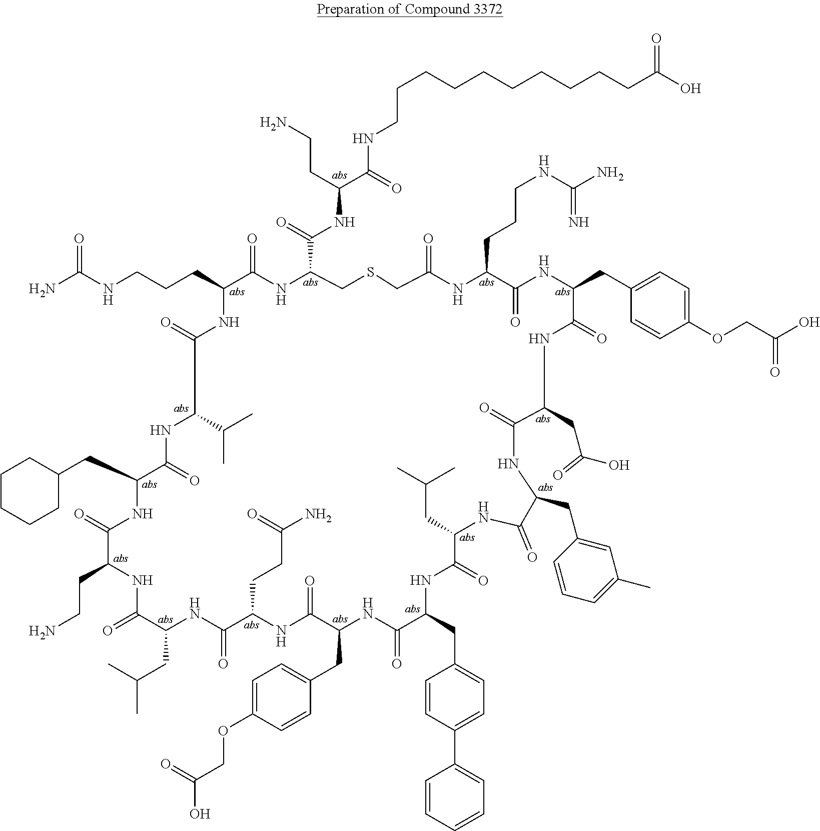 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(OCC(=O)O)cc3)NC(=O)[C@H](Cc3ccc(-c4ccccc4)cc3)NC(=O)[C@H](CC(C)C)NC2=O)c1]

Compound 3372 was prepared on a 50 μmol scale. The yield of the product was 12.7 mg, and its estimated purity by LCMS analysis was 95.8%. Analysis condition A: Retention time=1.6 min; ESI-MS(+) m/z [M+2H]²⁺: 1203.9.

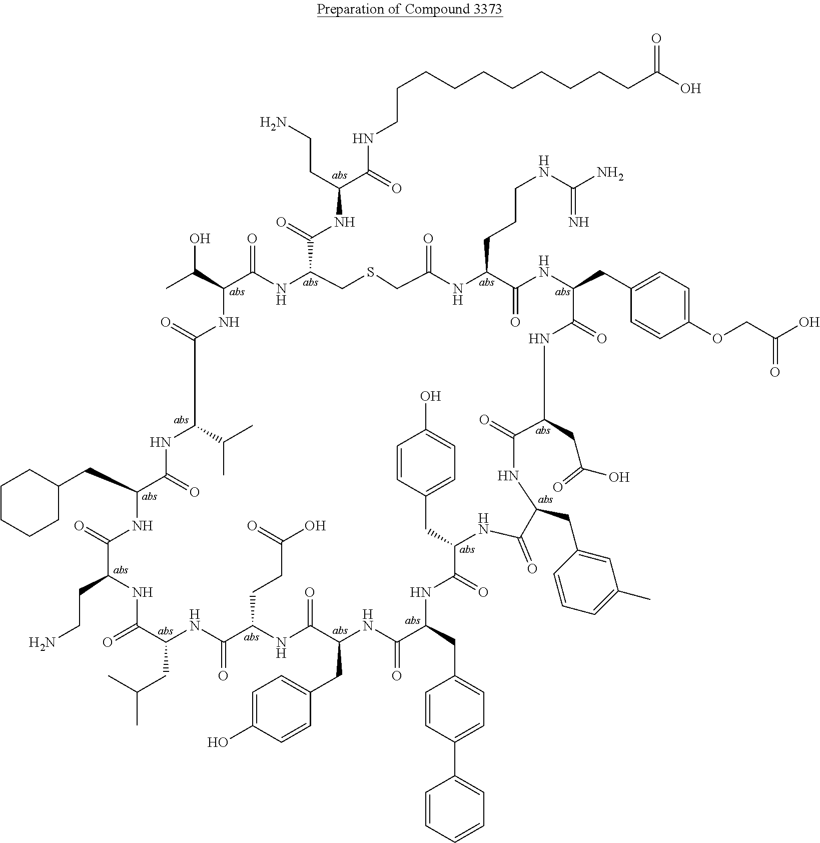 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCC(=O)O)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 3373 was prepared on a 50 μmol scale. The yield of the product was 17.1 mg, and its estimated purity by LCMS analysis was 95%. Analysis condition A: Retention time=1.65 min; ESI-MS(+) m/z [M+3H]³⁺: 782.2.

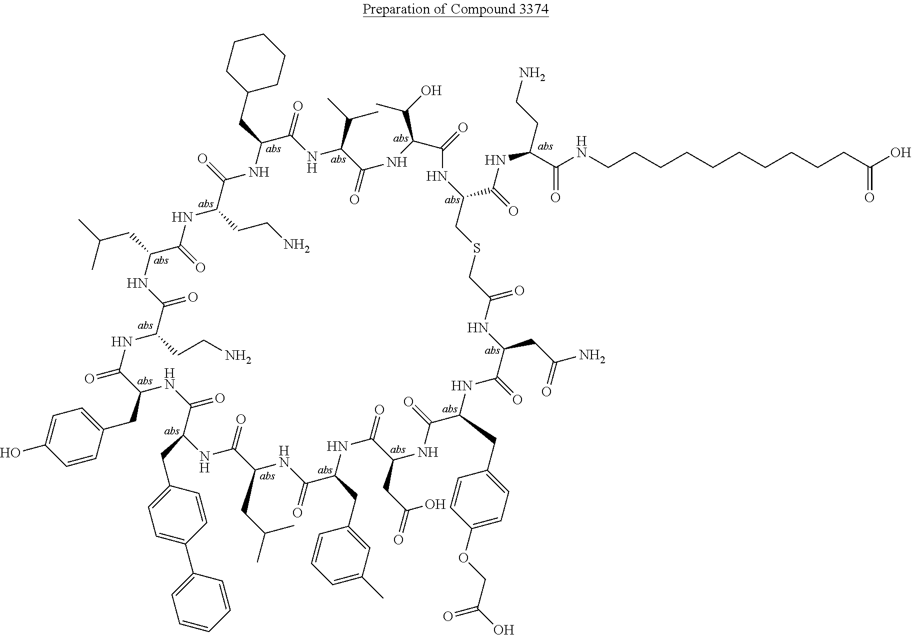 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(O)cc3)NC(=O)[C@H](Cc3ccc(-c4ccccc4)cc3)NC(=O)[C@H](CC(C)C)NC2=O)c1]

Compound 3374 was prepared on a 50 μmol scale. The yield of the product was 46.9 mg, and its estimated purity by LCMS analysis was 84.2%. Analysis condition B: Retention time=1.68 min; ESI-MS(+) m/z [M+2H]²⁺: 1112.1.

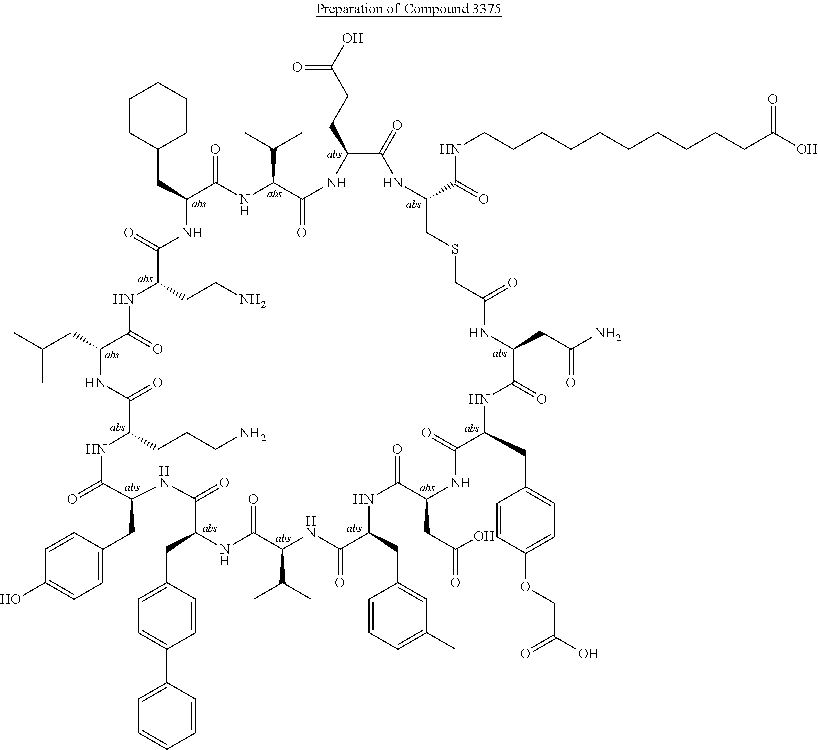 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3375 was prepared on a 50 μmol scale. The yield of the product was 31.8 mg, and its estimated purity by LCMS analysis was 94.6%. Analysis condition A: Retention time=1.59 min; ESI-MS(+) m/z [M+2H]²⁺: 1076.2.

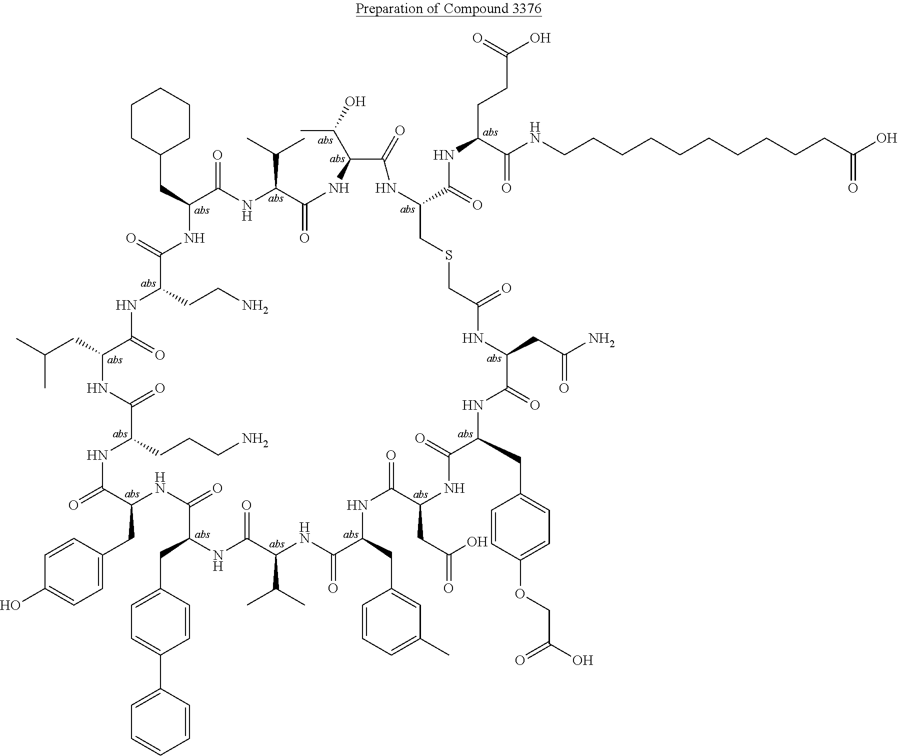 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCC(=O)O)C(=O)NCCCCCCCCCCC(=O)O)NC(=O)[C@H]([C@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3376 was prepared on a 50 μmol scale. The yield of the product was 19 mg, and its estimated purity by LCMS analysis was 95.5%. Analysis condition A: Retention time=1.52 min; ESI-MS(+) m/z [M+2H]²⁺: 1126.

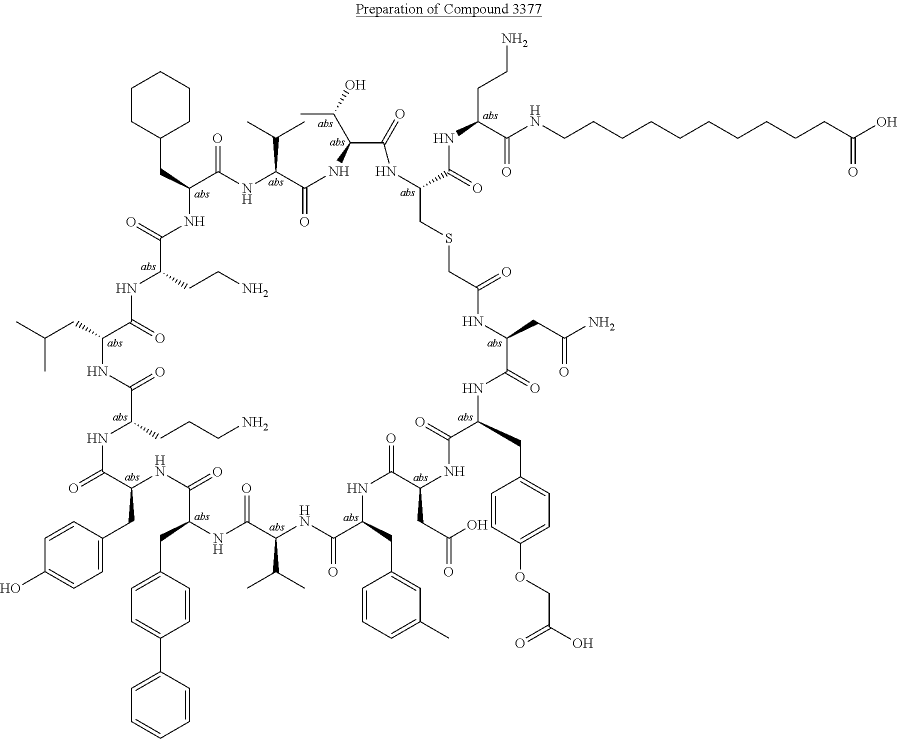 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H]([C@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3377 was prepared on a 50 μmol scale. The yield of the product was 35.5 mg, and its estimated purity by LCMS analysis was 96.1%. Analysis condition A: Retention time=1.55 min; ESI-MS(+) m/z [M+2H]²⁺: 1112.1.

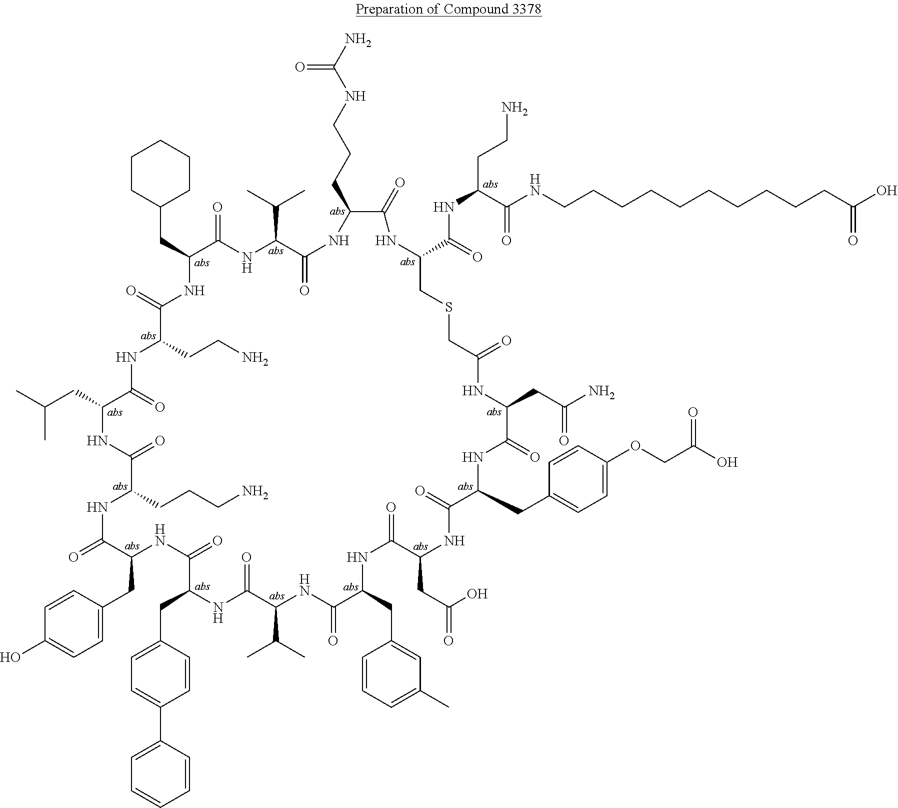 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3378 was prepared on a 50 μmol scale. The yield of the product was 21.1 mg, and its estimated purity by LCMS analysis was 95%. Analysis condition A: Retention time=1.61 min; ESI-MS(+) m/z [M+2H]²⁺: 1140.2.

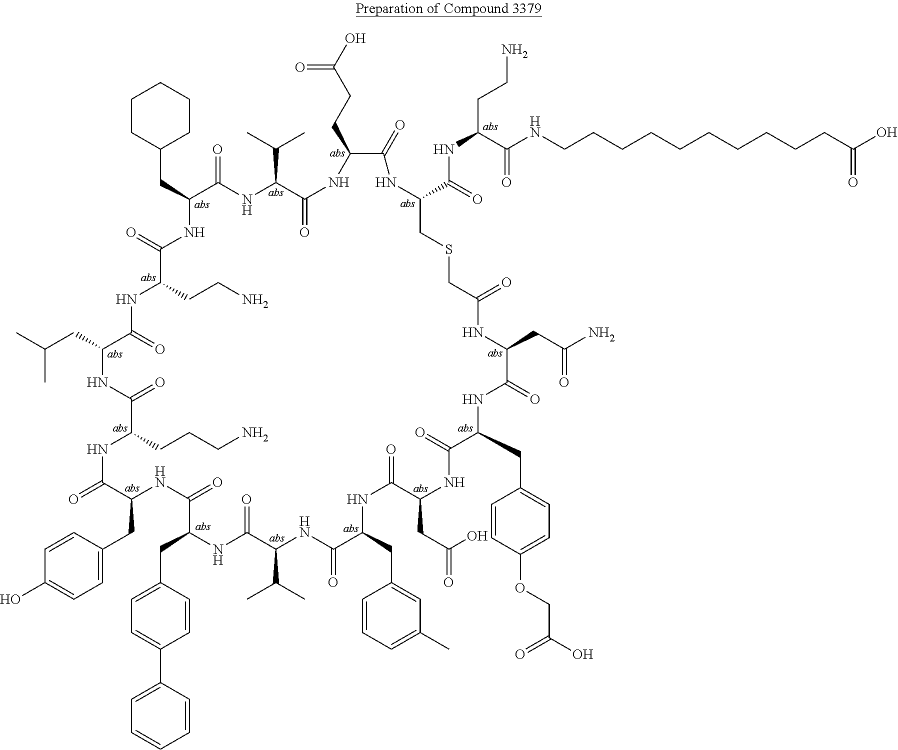 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3379 was prepared on a 50 μmol scale. The yield of the product was 15.9 mg, and its estimated purity by LCMS analysis was 95.4%. Analysis condition B: Retention time=1.63, 1.66 min; ESI-MS(+) m/z [M+2H]²⁺: 1126.04, 1126.04.

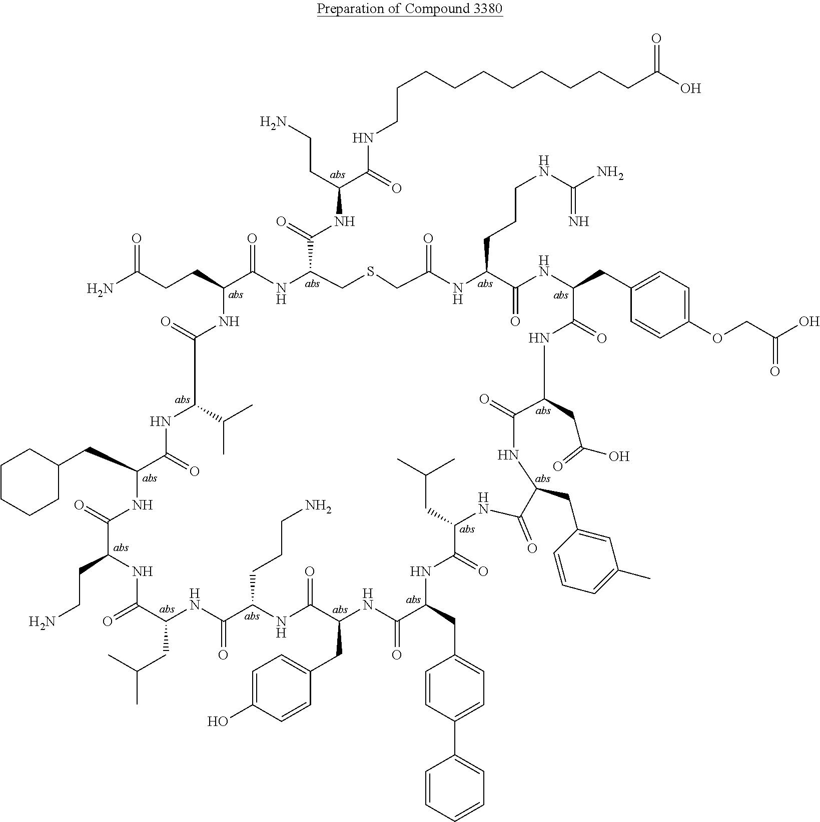 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CC(C)C)NC2=O)c1]

Compound 3380 was prepared on a 50 μmol scale. The yield of the product was 50.7 mg, and its estimated purity by LCMS analysis was 92.2%. Analysis condition A: Retention time=1.67 min; ESI-MS(+) m/z [M+3H]³⁺: 769.1.

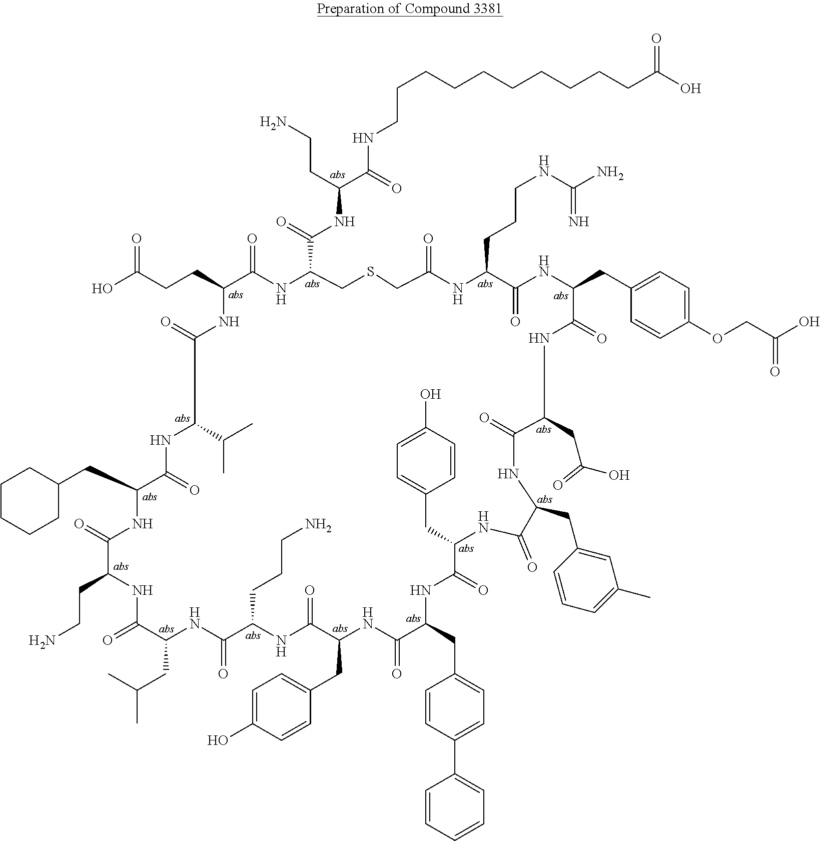 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 3381 was prepared on a 50 μmol scale. The yield of the product was 70 mg, and its estimated purity by LCMS analysis was 94.9%. Analysis condition B: Retention time=1.46 min; ESI-MS(+) m/z [M+3H]³⁺: 786.4.

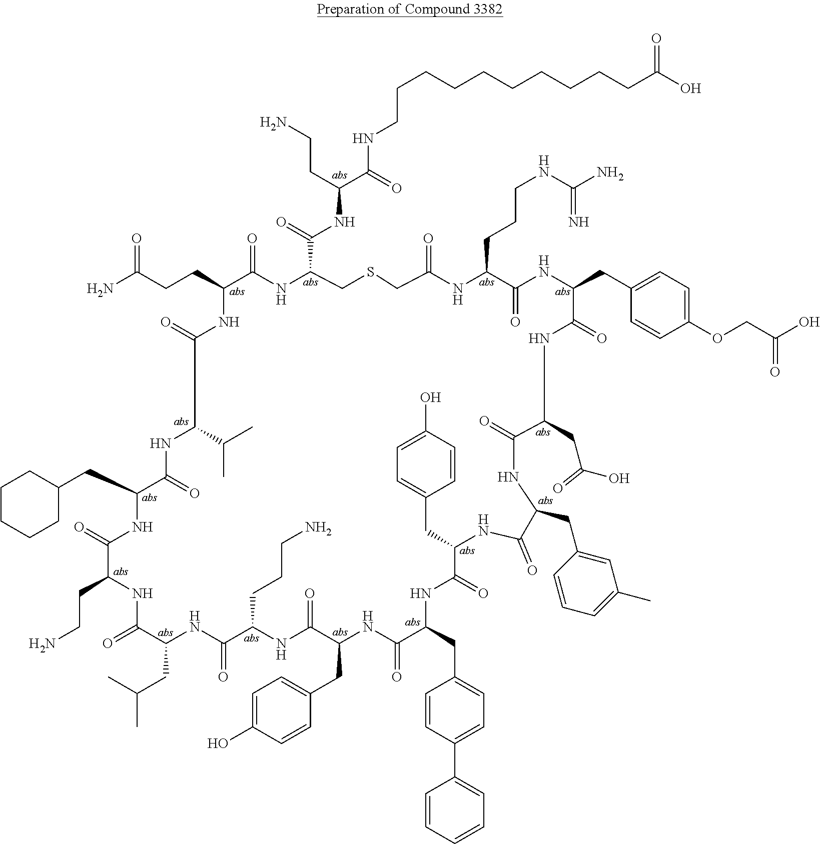 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 3382 was prepared on a 50 μmol scale. The yield of the product was 31.4 mg, and its estimated purity by LCMS analysis was 95.8%. Analysis condition B: Retention time-1.57 min; ESI-MS(+) m/z [M+2H]²⁺: 1179.

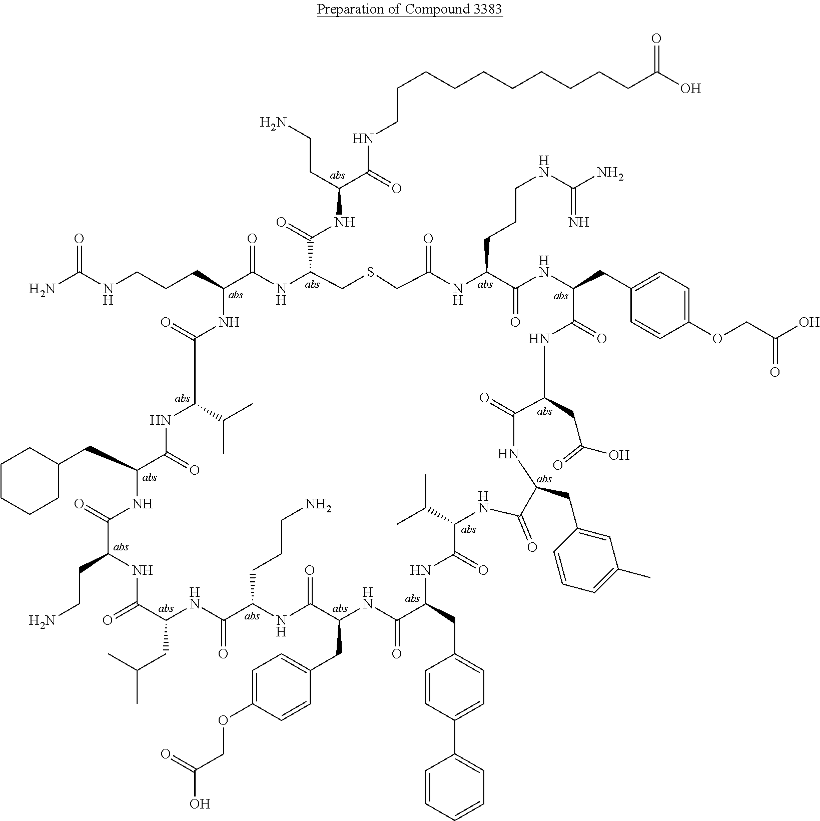 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 3383 was prepared on a 50 μmol scale. The yield of the product was 7.4 mg, and its estimated purity by LCMS analysis was 95.7%. Analysis condition A: Retention time=1.45 min; ESI-MS(+) m/z [M+2H]²⁺: 1190.3.

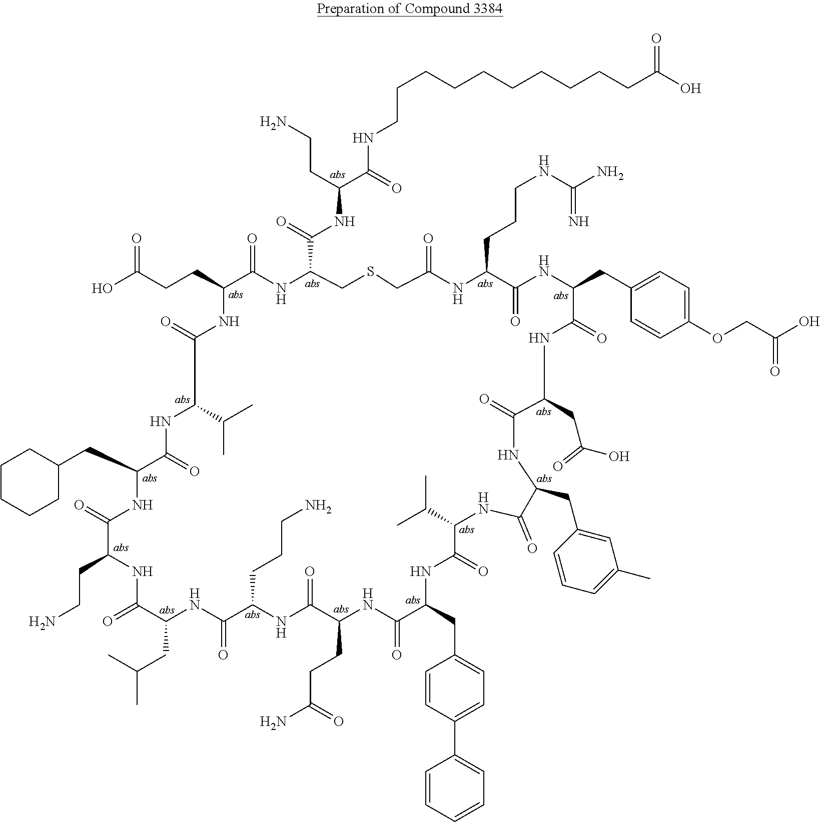 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 3384 was prepared on a 50 μmol scale. The yield of the product was 17.3 mg, and its estimated purity by LCMS analysis was 95.1%. Analysis condition A: Retention time=1.58 min; ESI-MS(+) m/z [M+2H]²⁺: 1129.3.

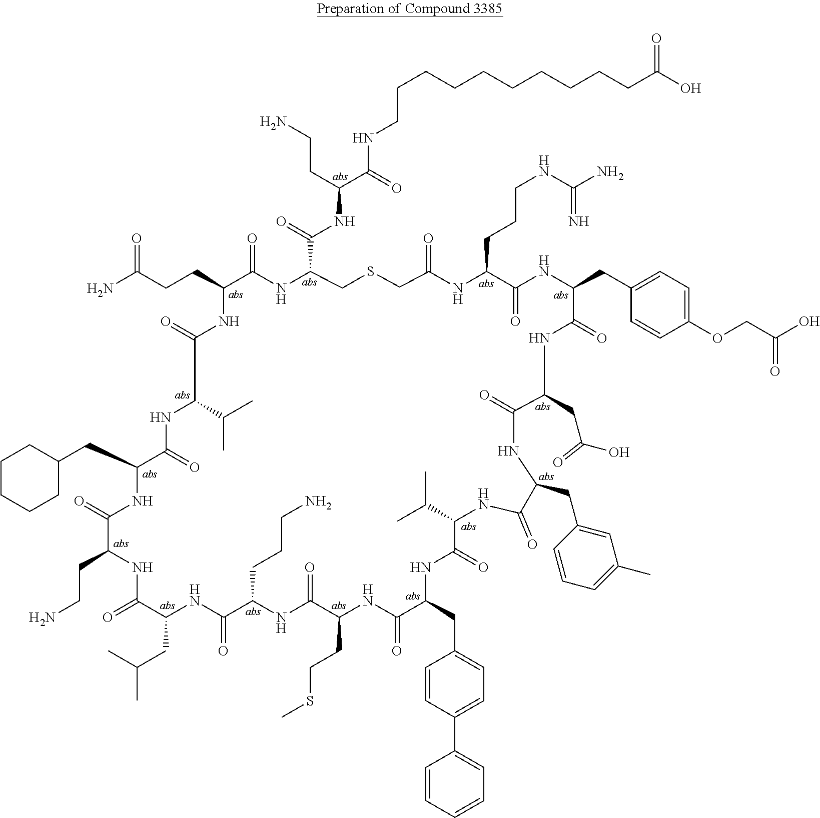 [SMILES: CSCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3385 was prepared on a 50 μmol scale. The yield of the product was 3.7 mg, and its estimated purity by LCMS analysis was 96.5%. Analysis condition A: Retention time=1.7 min; ESI-MS(+) m/z [M+2H]²⁺: 1131.1.

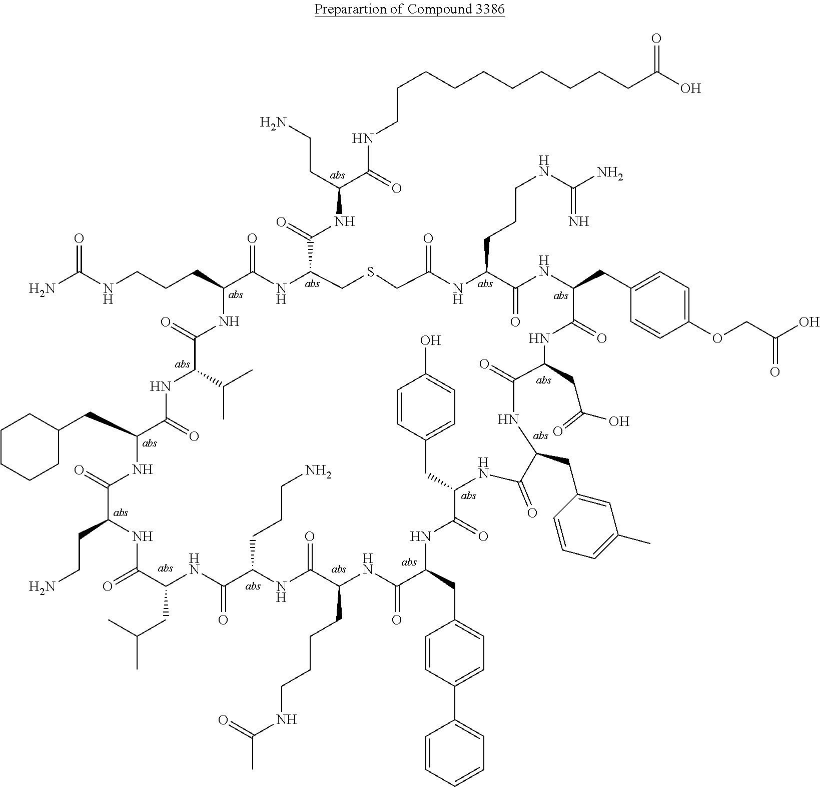 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3386 was prepared on a 50 μmol scale. The yield of the product was 14.8 mg, and its estimated purity by LCMS analysis was 99.2%. Analysis condition A: Retention time=1.48 min; ESI-MS(+) m/z [M+3H]³⁺: 798.

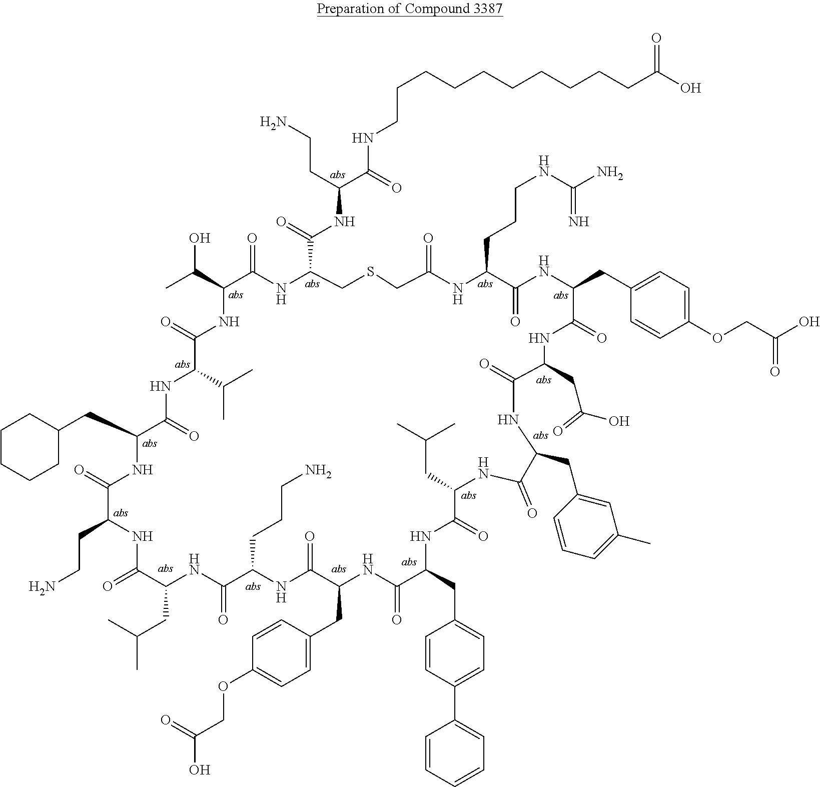 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](CC(C)C)NC2=O)c1]

Compound 3387 was prepared on a 50 μmol scale. The yield of the product was 16.5 mg, and its estimated purity by LCMS analysis was 96%. Analysis condition B: Retention time=1.59 min; ESI-MS(+) m/z [M+3H]³⁺: 779.9.

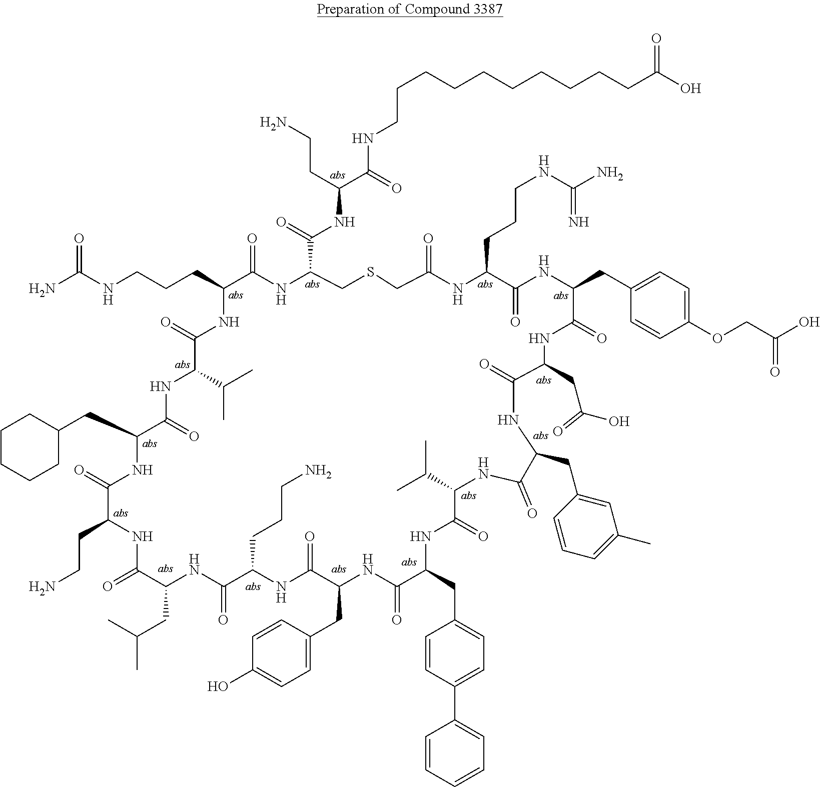 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3388 was prepared on a 50 μmol scale. The yield of the product was 40.9 mg, and its estimated purity by LCMS analysis was 96.5%. Analysis condition A: Retention time=1.5 min; ESI-MS(+) m/z [M+3H]³⁺: 779.

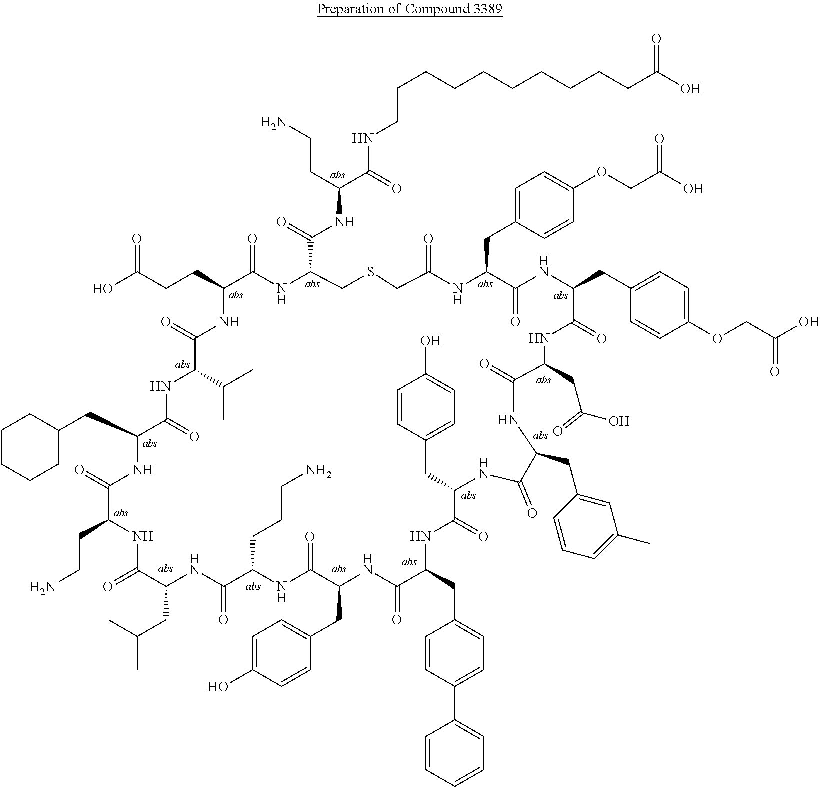 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 3389 was prepared on a 50 μmol scale. The yield of the product was 10.2 mg, and its estimated purity by LCMS analysis was 100%. Analysis condition A: Retention time=1.66 min; ESI-MS(+) m/z [M+3H]³⁺: 803.3.

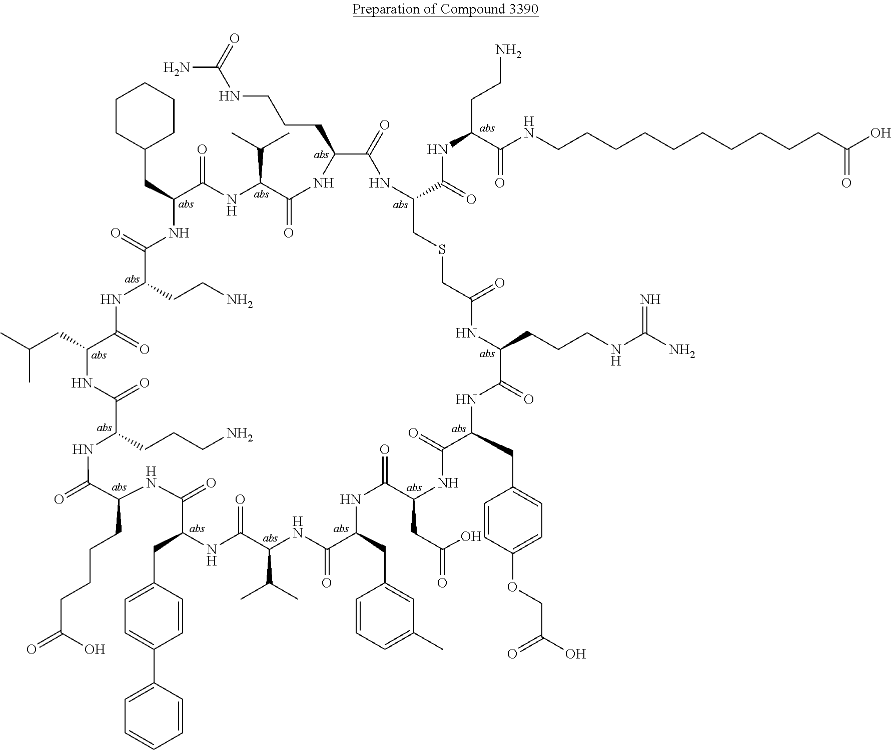 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCCC(=O)O)NC(=O)[C@H](Cc3ccc(-c4ccccc4)cc3)NC(=O)[C@H](C(C)C)NC2=O)c1]

Compound 3390 was prepared on a 50 μmol scale. The yield of the product was 8.9 mg, and its estimated purity by LCMS analysis was 95%. Analysis condition B: Retention time=1.51 min; ESI-MS(+) m/z [M+2H]²⁺: 1158.1.

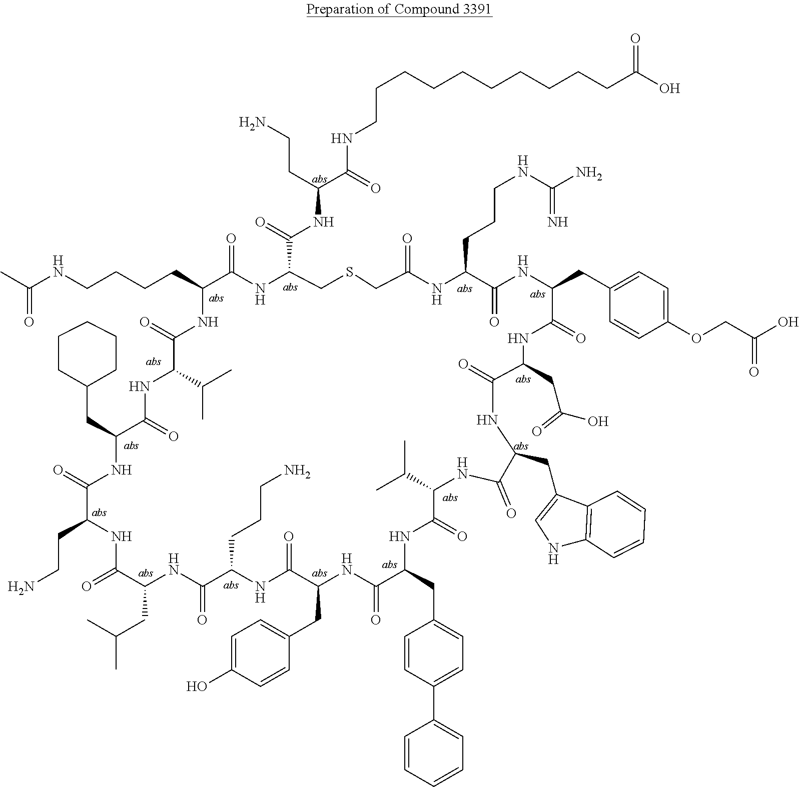 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC1=O]

Compound 3391 was prepared on a 50 μmol scale. The yield of the product was 12.2 mg, and its estimated purity by LCMS analysis was 96.3%. Analysis condition A: Retention time=1.61 min; ESI-MS(+) m/z [M+2H]²⁺: 1179.9.

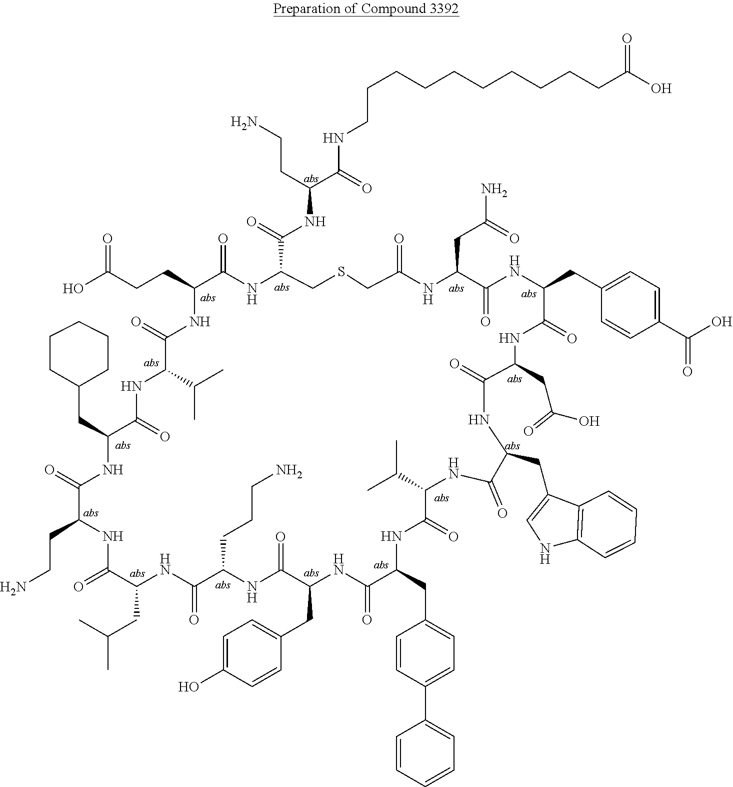 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CC(N)=O)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3392 was prepared on a 50 μmol scale. The yield of the product was 15.5 mg, and its estimated purity by LCMS analysis was 100%. Analysis condition A: Retention time=1.66 min; ESI-MS(+) m/z [M+2H]²⁺: 1123.3.

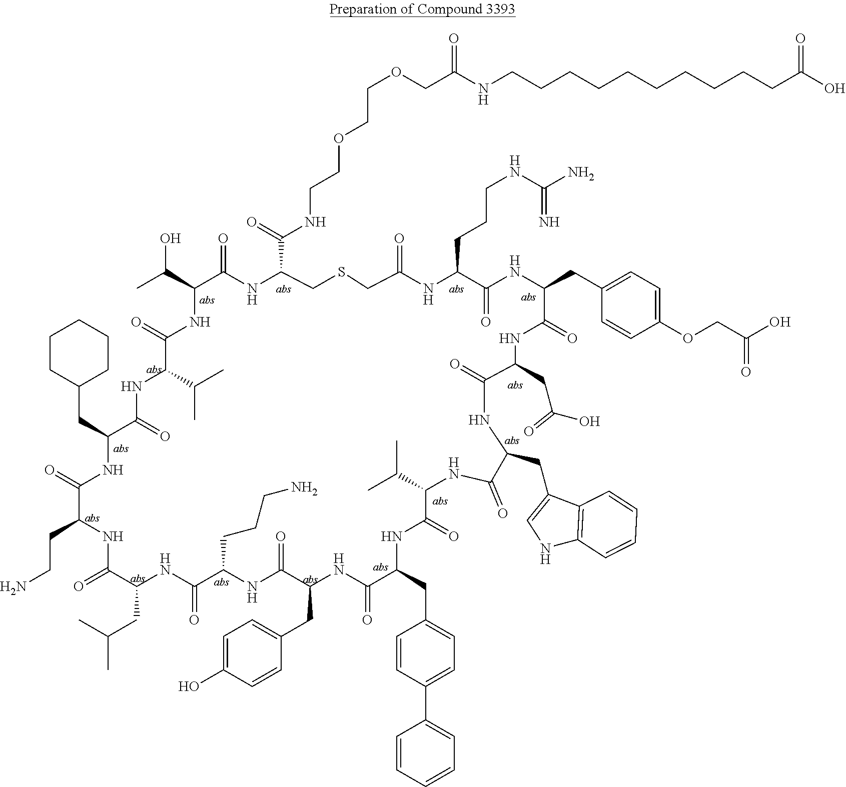 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3393 was prepared on a 50 μmol scale. The yield of the product was 47.2 mg, and its estimated purity by LCMS analysis was 87.7%. Analysis condition B: Retention time=1.67 min; ESI-MS(+) m/z [M+2H]²⁺: 1168.1.

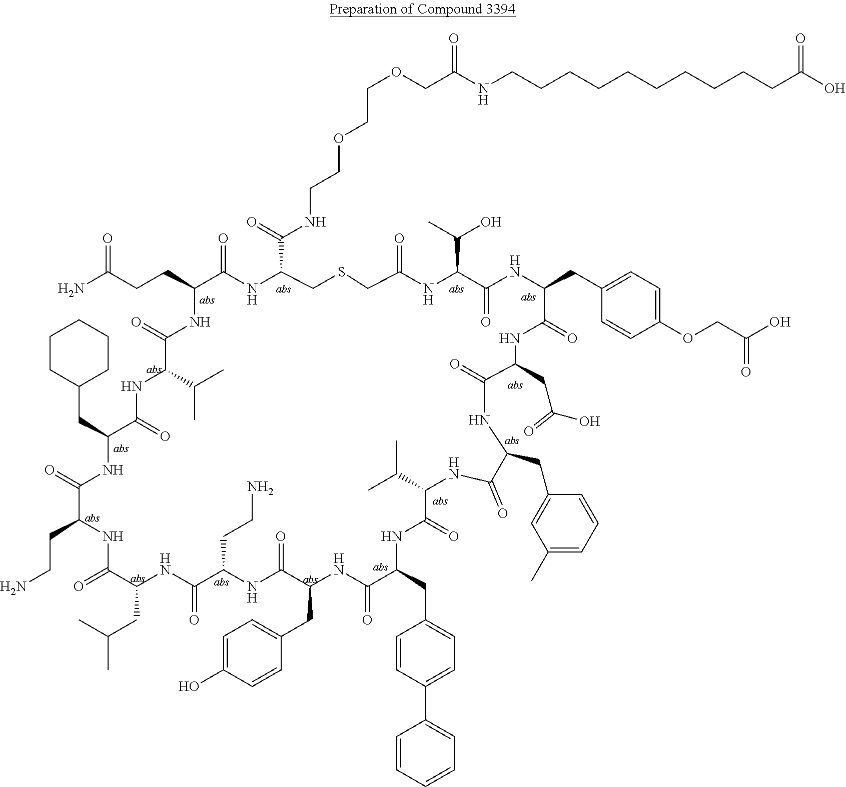 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3394 was prepared on a 50 μmol scale. The yield of the product was 98.5 mg, and its estimated purity by LCMS analysis was 95.5%. Analysis condition A: Retention time=1.73 min; ESI-MS(+) m/z [M+2H]²⁺: 1134.3.

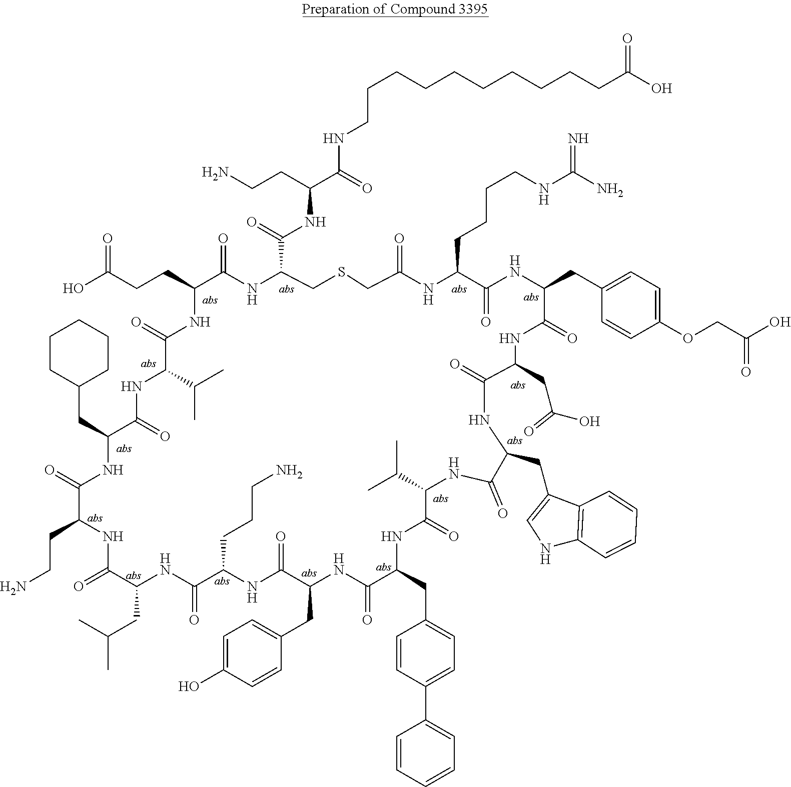 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3395 was prepared on a 50 μmol scale. The yield of the product was 10.8 mg, and its estimated purity by LCMS analysis was 100%. Analysis condition A: Retention time=1.63 min; ESI-MS(+) m/z [M+3H] 3:778.1.

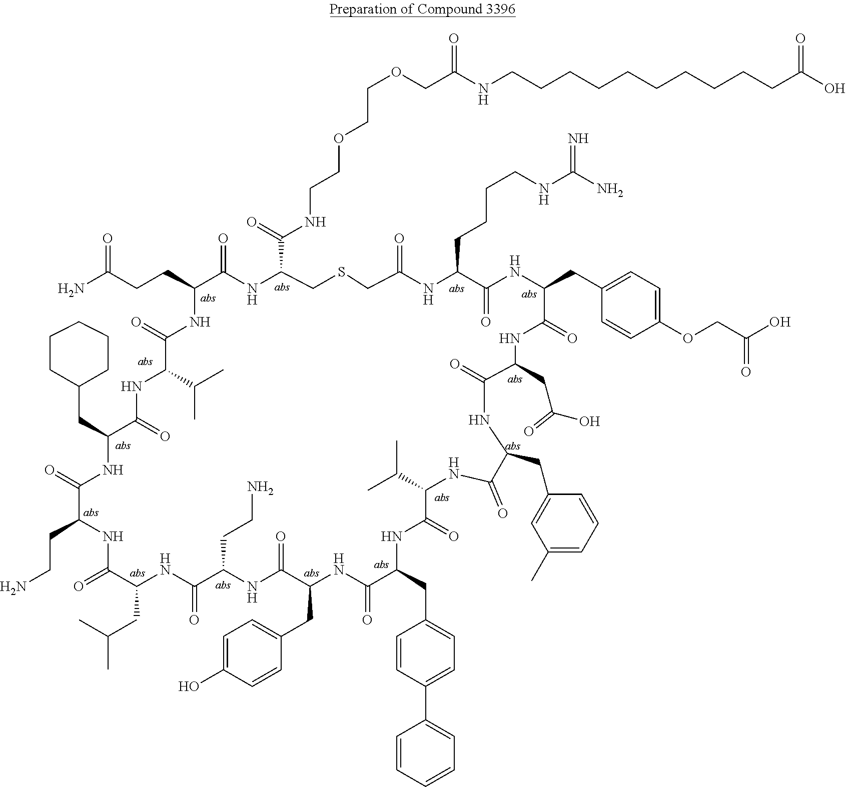 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)NCCOCCOCC(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3396 was prepared on a 50 μmol scale. The yield of the product was 35.5 mg, and its estimated purity by LCMS analysis was 100%. Analysis condition A: Retention time=1.71 min; ESI-MS(+) m/z [M+2H]²⁺: 1169.

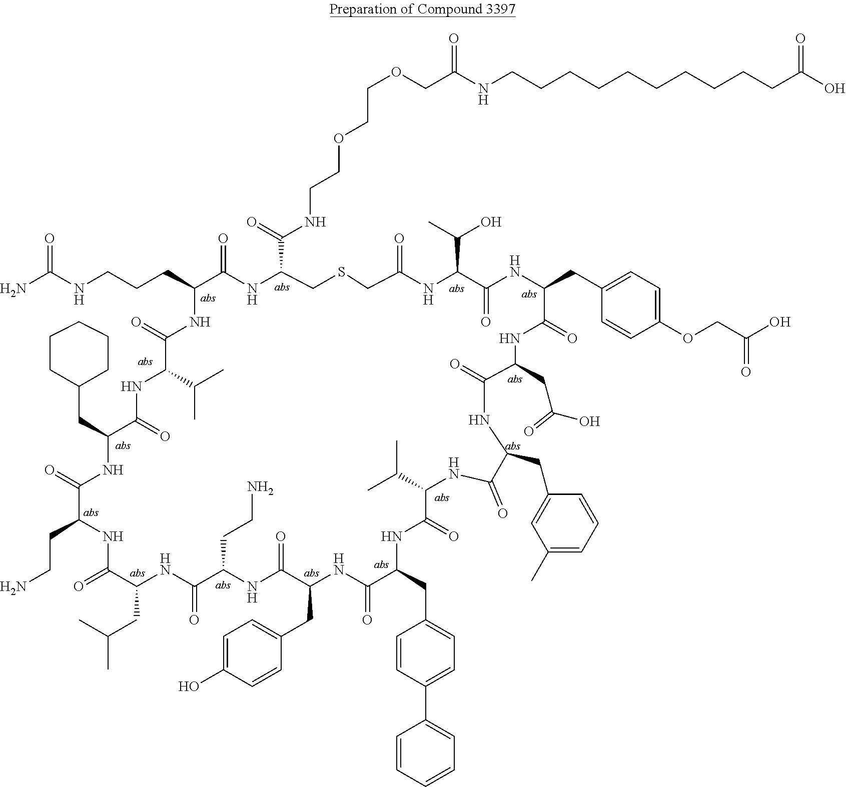 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3397 was prepared on a 50 μmol scale. The yield of the product was 28.9 mg, and its estimated purity by LCMS analysis was 97.6%. Analysis condition A: Retention time=1.7 min; ESI-MS(+) m/z [M+2H]²⁺: 1149.1.

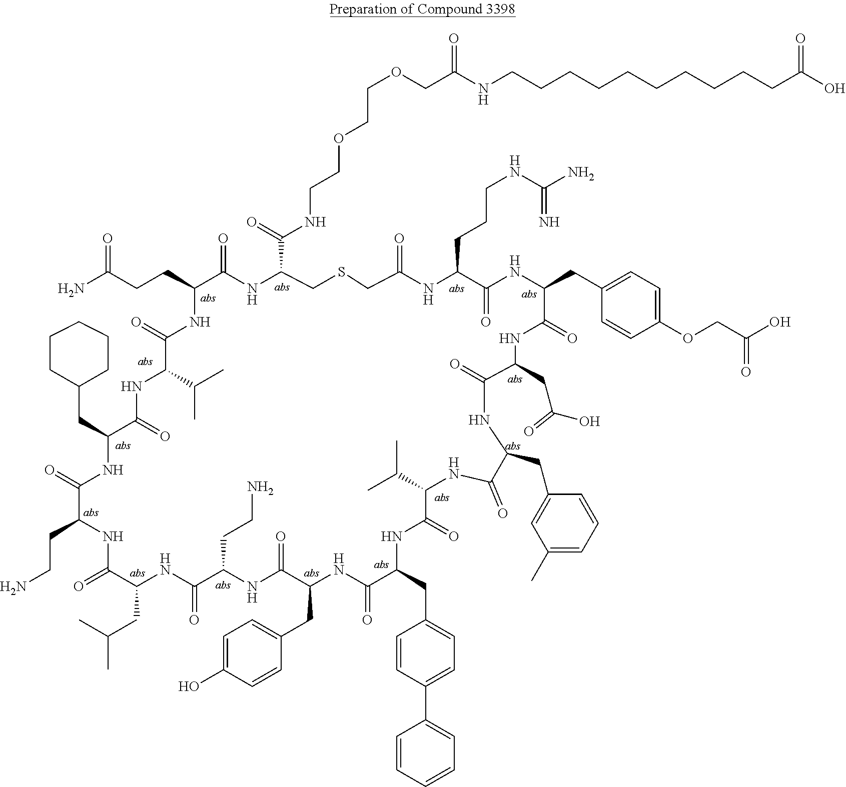 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3398 was prepared on a 50 μmol scale. The yield of the product was 87.4 mg, and its estimated purity by LCMS analysis was 89.3%. Analysis condition B: Retention time=1.67 min; ESI-MS(+) m/z [M+2H]²⁺: 1162.2.

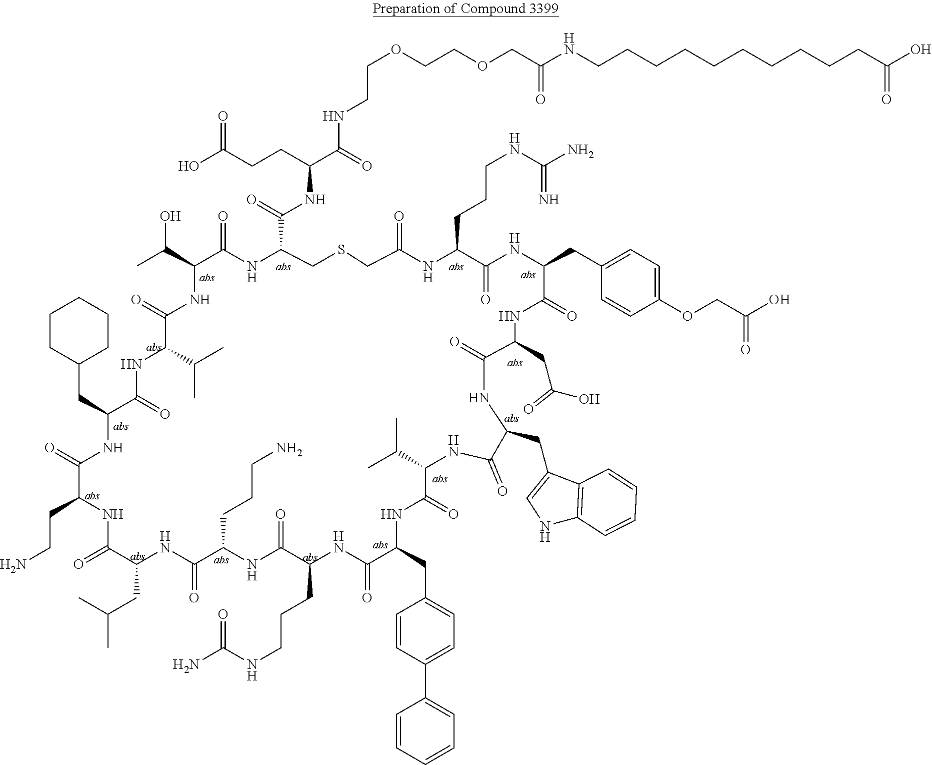 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCC(=O)O)C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3399 was prepared on a 50 μmol scale. The yield of the product was 15.8 mg, and its estimated purity by LCMS analysis was 95.4%. Analysis condition A: Retention time=1.57 min; ESI-MS(+) m/z [M+3H]³⁺: 820.1.

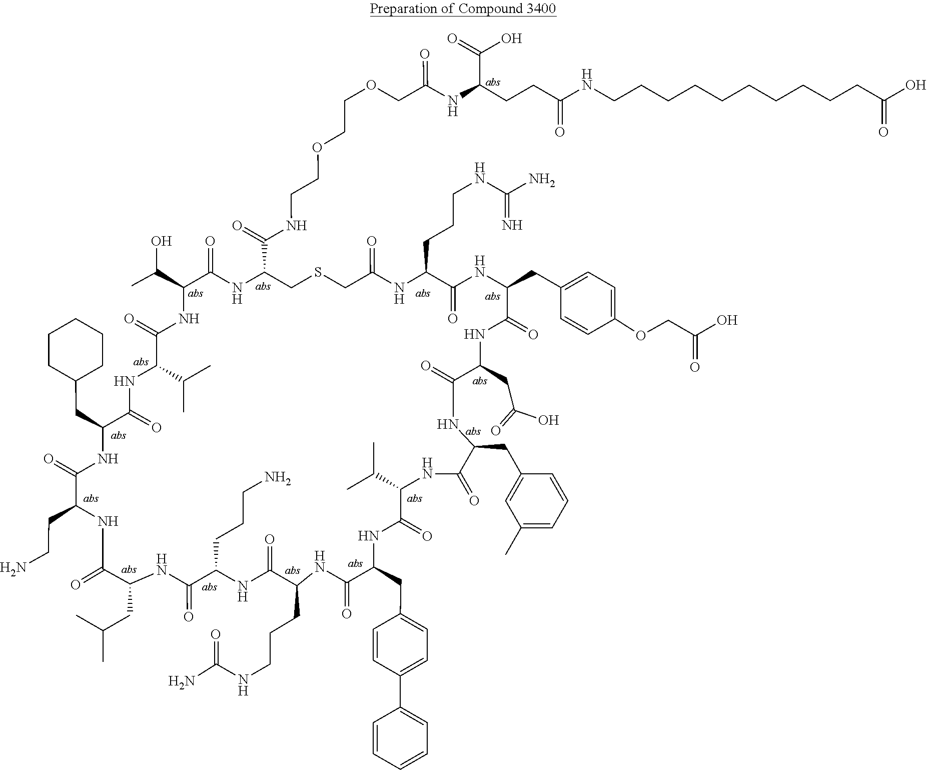 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)N[C@H](CCC(=O)NCCCCCCCCCCC(=O)O)C(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3400 was prepared on a 50 μmol scale. The yield of the product was 48 mg, and its estimated purity by LCMS analysis was 100%. Analysis condition A: Retention time=1.59 min; ESI-MS(+) m/z [M+2H]²⁺: 1217.1.

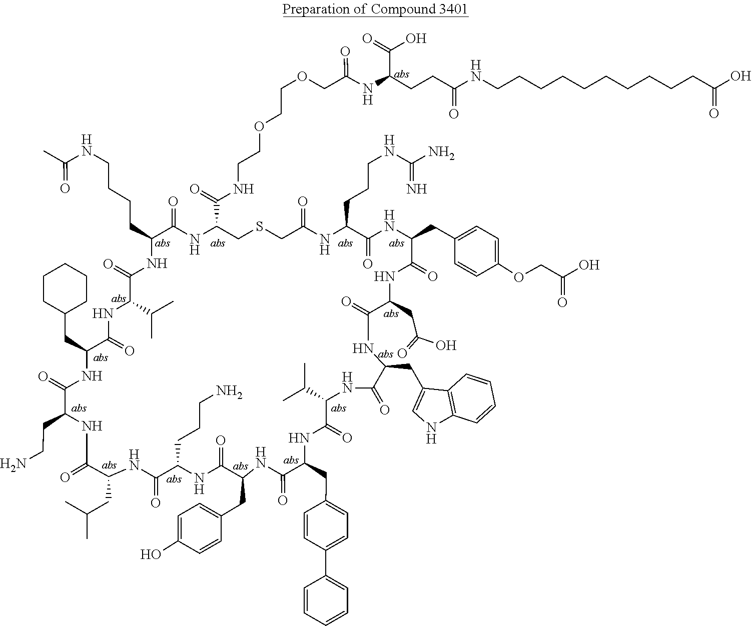 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCOCCOCC(=O)N[C@H](CCC(=O)NCCCCCCCCCCC(=O)O)C(=O)O)NC1=O]

Compound 3401 was prepared on a 50 μmol scale. The yield of the product was 9.4 mg, and its estimated purity by LCMS analysis was 97.4%. Analysis condition A: Retention time=1.55 min; ESI-MS(+) m/z [M+2H]²⁺: 1266.9.

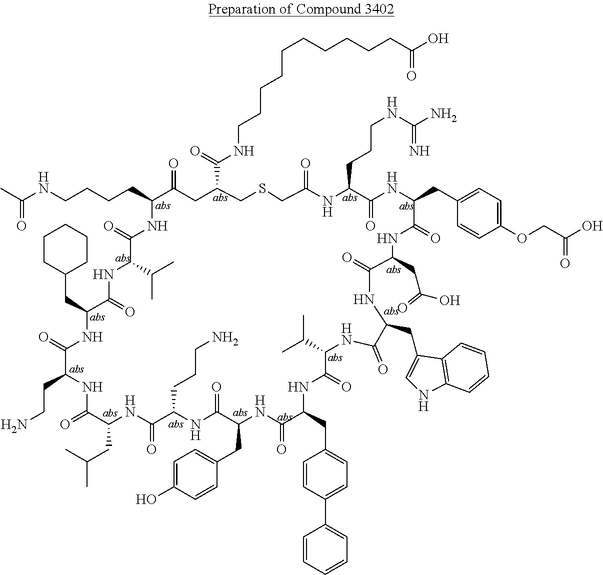 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)NCCCCCCCCCCC(=O)O)CC1=O]

Compound 3402 was prepared on a 50 μmol scale. The yield of the product was 38.9 mg, and its estimated purity by LCMS analysis was 96.9%. Analysis condition B: Retention time=1.58 min; ESI-MS(+) m/z [M+2H]²⁺: 1129.9.

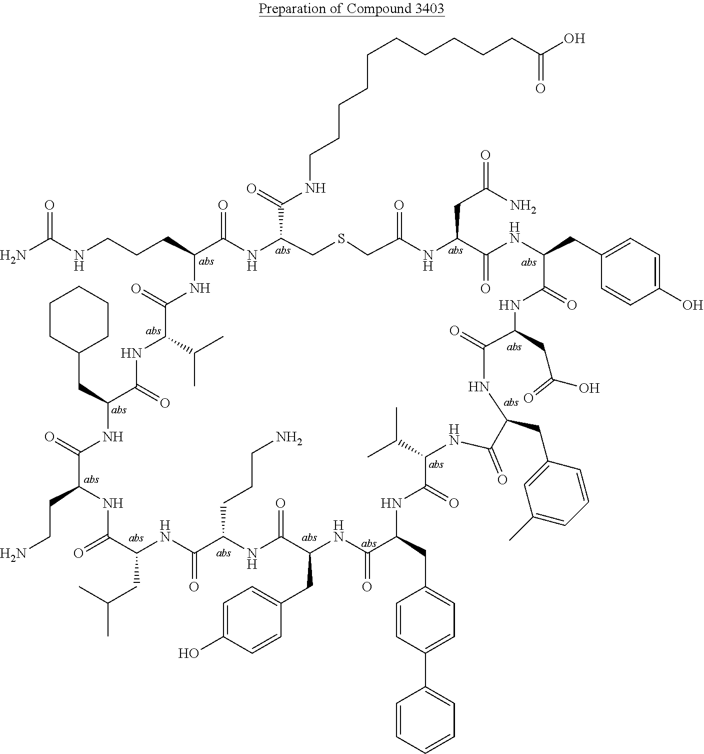 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3403 was prepared on a 50 μmol scale. The yield of the product was 19.7 mg, and its estimated purity by LCMS analysis was 100%. Analysis condition A: Retention time=1.85 min; ESI-MS(+) m/z [M+2H]²⁺: 1061.

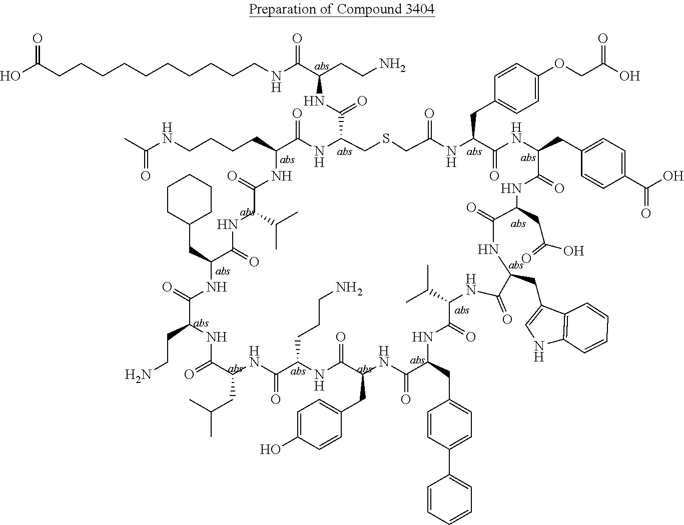 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@H](CCN)C(=O)NCCCCCCCCCCC(=O)O)NC1=O]

Compound 3404 was prepared on a 50 μmol scale. The yield of the product was 27.1 mg, and its estimated purity by LCMS analysis was 90.3%. Analysis condition A: Retention time=1.55 min; ESI-MS(+) m/z [M+2H]²⁺: 1197.9.

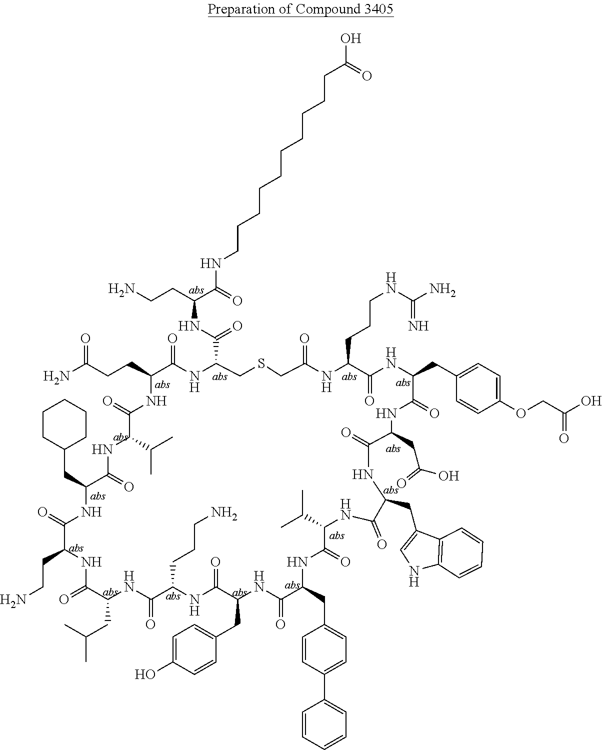 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC1=O]

Compound 3405 was prepared on a 50 μmol scale. The yield of the product was 44.7 mg, and its estimated purity by LCMS analysis was 100%. Analysis condition A: Retention time=1.51 min; ESI-MS(+) m/z [M+2H]²⁺: 1159.1.

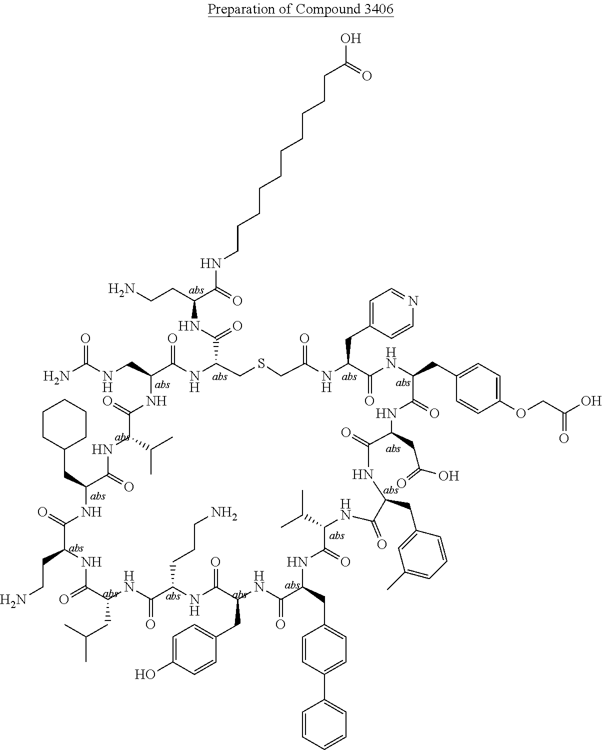 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3406 was prepared on a 50 μmol scale. The yield of the product was 16.8 mg, and its estimated purity by LCMS analysis was 96.7%. Analysis condition B: Retention time=1.53 min; ESI-MS(+) m/z [M+2H]²⁺: 1156.8.

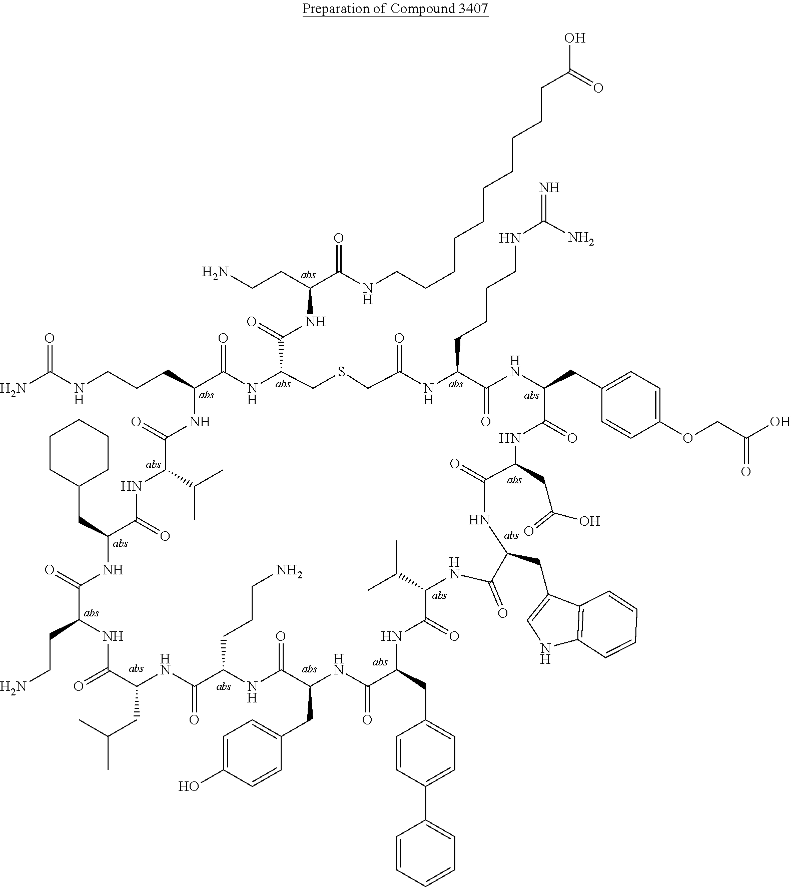 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC1=O]

Compound 3407 was prepared on a 50 μmol scale. The yield of the product was 19.3 mg, and its estimated purity by LCMS analysis was 97.2%. Analysis condition B: Retention time=1.47 min; ESI-MS(+) m/z [M+2H]²⁺: 1180.4.

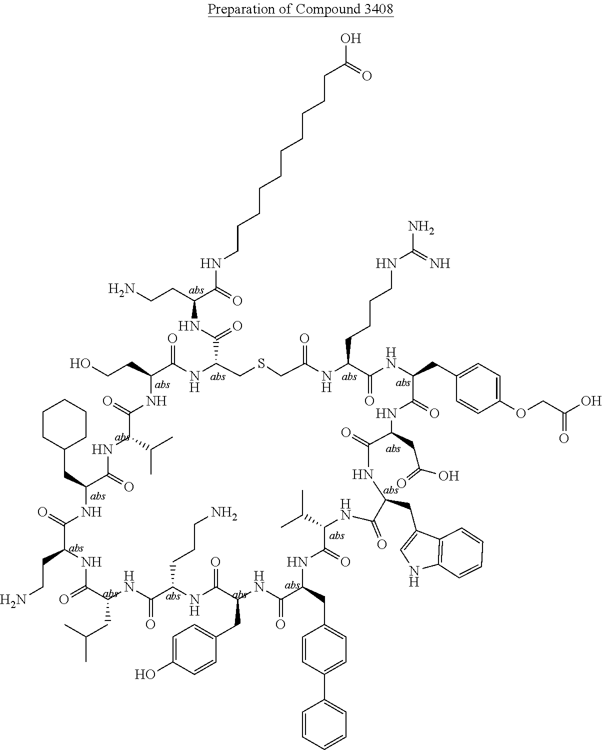 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC1=O]

Compound 3408 was prepared on a 50 μmol scale. The yield of the product was 6.1 mg, and its estimated purity by LCMS analysis was 100%. Analysis condition A: Retention time=1.49 min; ESI-MS(+) m/z [M+2H]²⁺: 1152.9.

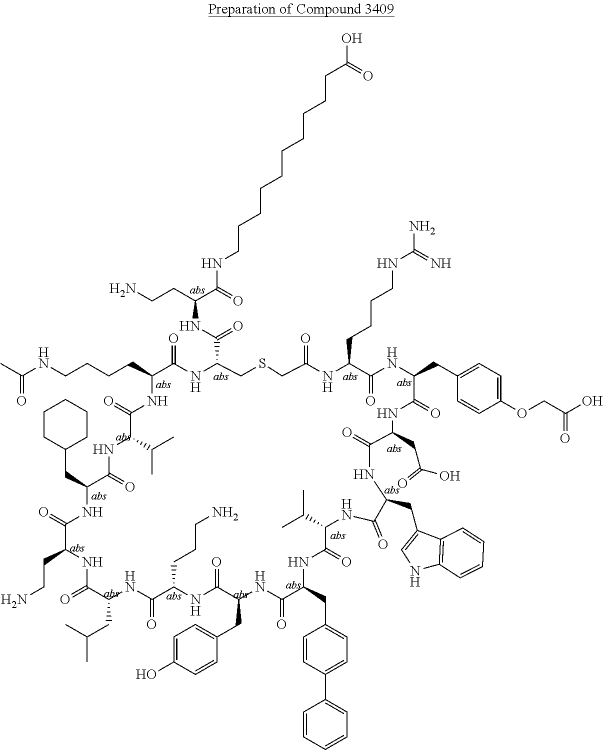 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC1=O]

Compound 3409 was prepared on a 50 μmol scale. The yield of the product was 14.4 mg, and its estimated purity by LCMS analysis was 95.7%. Analysis condition B: Retention time=1.55 min; ESI-MS(+) m/z [M+2H]²⁺: 1187.3.

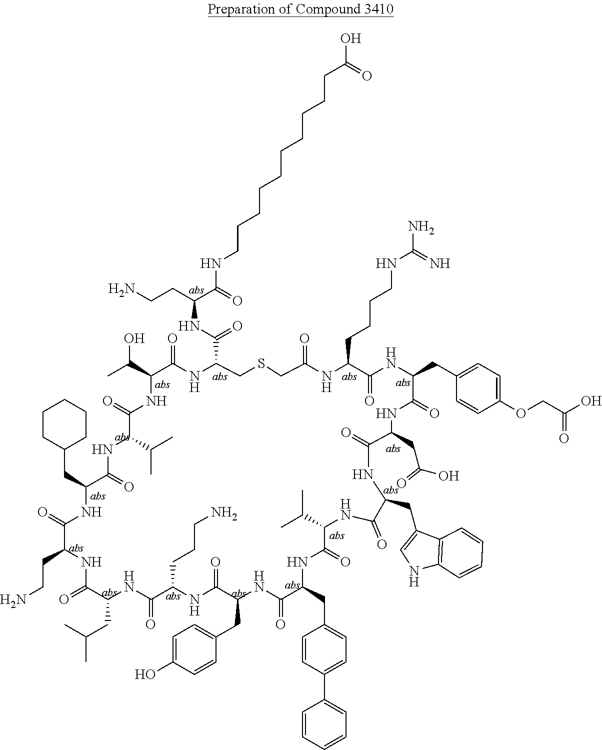 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3410 was prepared on a 50 μmol scale. The yield of the product was 26.8 mg, and its estimated purity by LCMS analysis was 97.7%. Analysis condition B: Retention time=1.57 min; ESI-MS(+) m/z [M+3H]³⁺: 769.1.

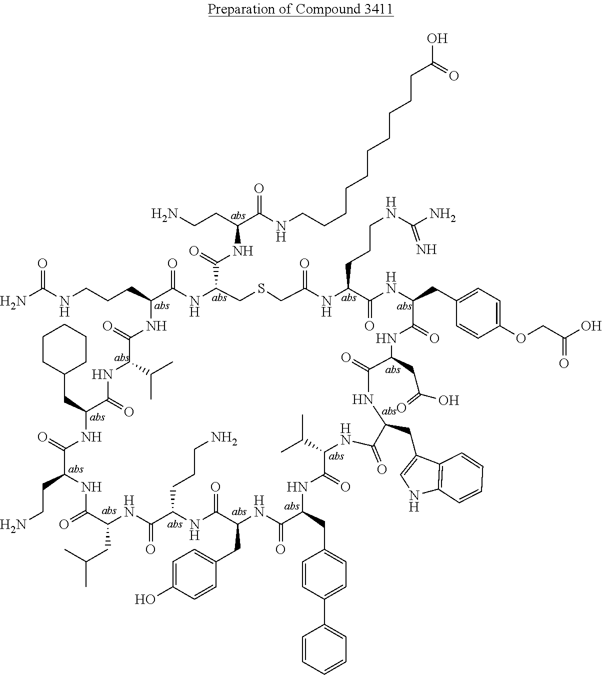 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCCNC(N)=O)NC(=O)[C@H](C(C)C)NC(=O)[C@H](CC2CCCCC2)NC(=O)[C@H](CCN)NC1=O]

Compound 3411 was prepared on a 50 μmol scale. The yield of the product was 6.6 mg, and its estimated purity by LCMS analysis was 98.7%. Analysis condition A: Retention time=1.59 min; ESI-MS(+) m/z [M+2H]²⁺: 783.1

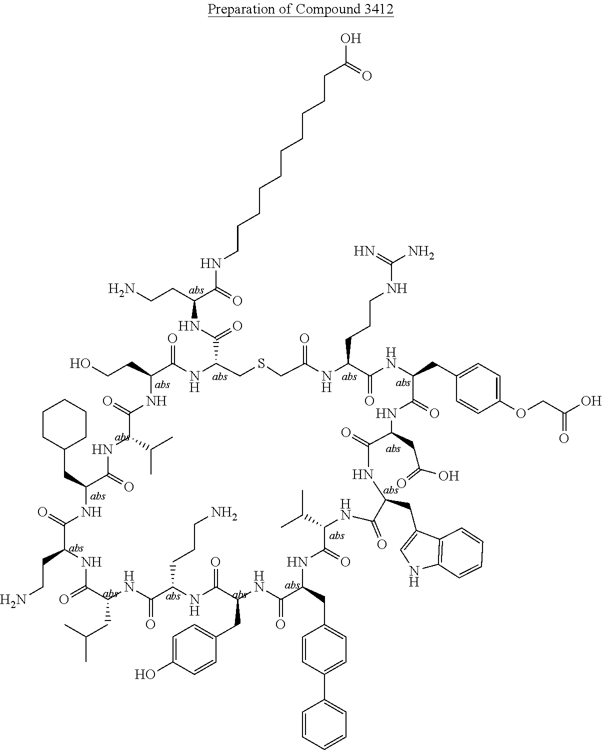 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC1=O]

Compound 3412 was prepared on a 50 μmol scale. The yield of the product was 10.2 mg, and its estimated purity by LCMS analysis was 90.2%. Analysis condition B: Retention time=1.56 min; ESI-MS(+) m/z [M+2H]²⁺: 1145.3.

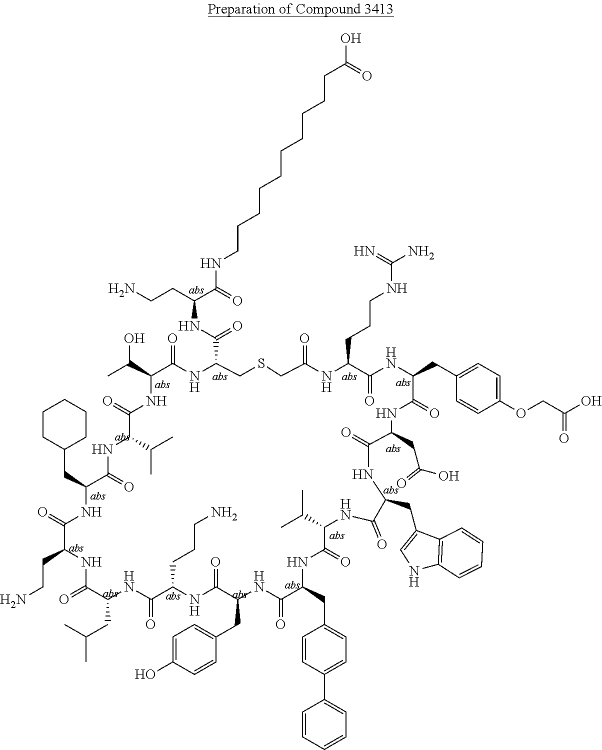 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3413 was prepared on a 50 μmol scale. The yield of the product was 24.5 mg, and its estimated purity by LCMS analysis was 96.2%. Analysis condition B: Retention time=1.52 min; ESI-MS(+) m/z [M+3H]³⁺: 764.3.

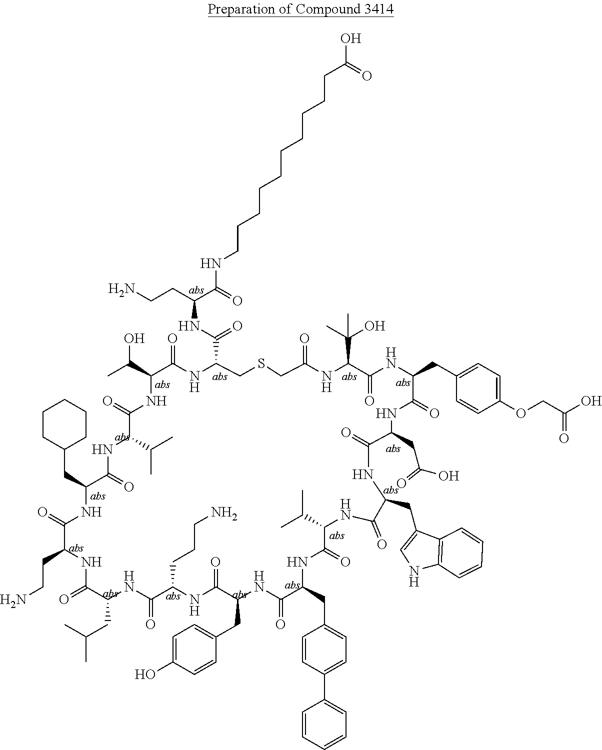 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3414 was prepared on a 50 μmol scale. The yield of the product was 11.3 mg, and its estimated purity by LCMS analysis was 99.1%. Analysis condition B: Retention time=1.65 min; ESI-MS(+) m/z [M+2H]²⁺: 1125.2.

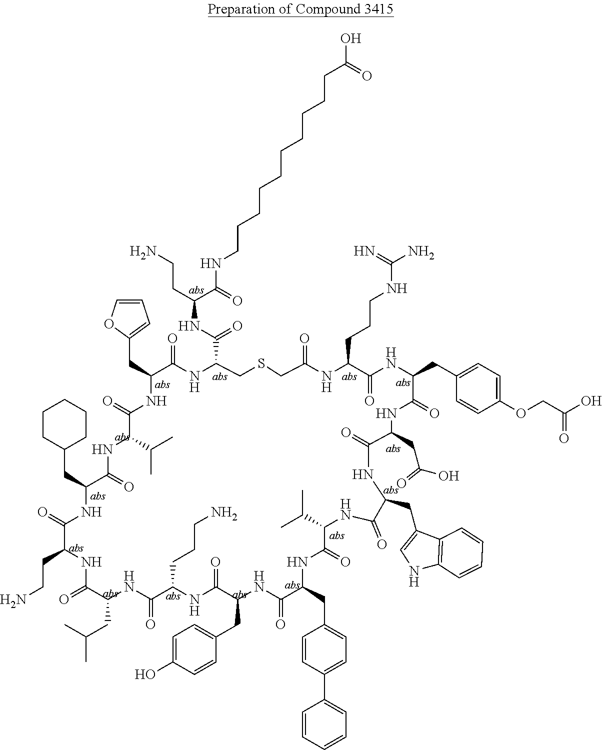 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](Cc2ccco2)NC(=O)[C@H](C(C)C)NC(=O)[C@H](CC2CCCCC2)NC(=O)[C@H](CCN)NC1=O]

Compound 3415 was prepared on a 50 μmol scale. The yield of the product was 12.8 mg, and its estimated purity by LCMS analysis was 97%. Analysis condition A: Retention time=1.69 min; ESI-MS(+) m/z [M+3H]³⁺: 776.4.

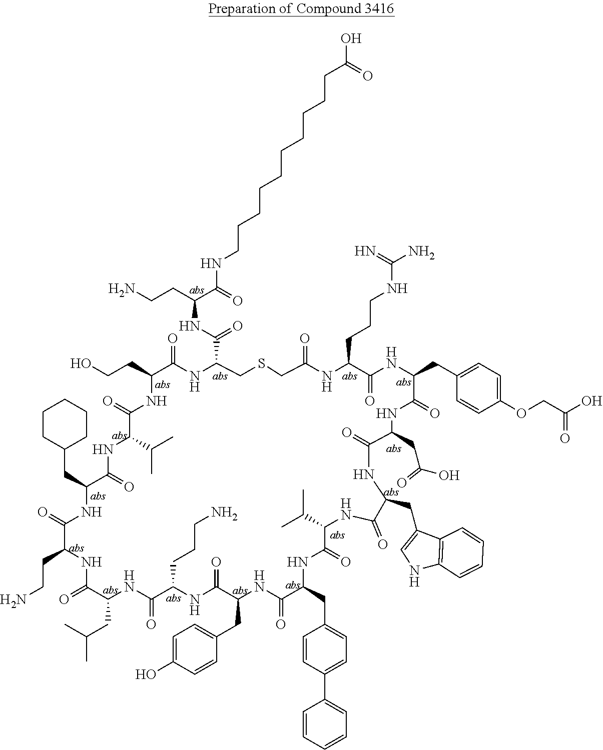 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC1=O]

Compound 3416 was prepared on a 50 μmol scale. The yield of the product was 15.7 mg, and its estimated purity by LCMS analysis was 100%. Analysis condition A: Retention time=1.56 min; ESI-MS(+) m/z [M+2H]²⁺: 1165.9.

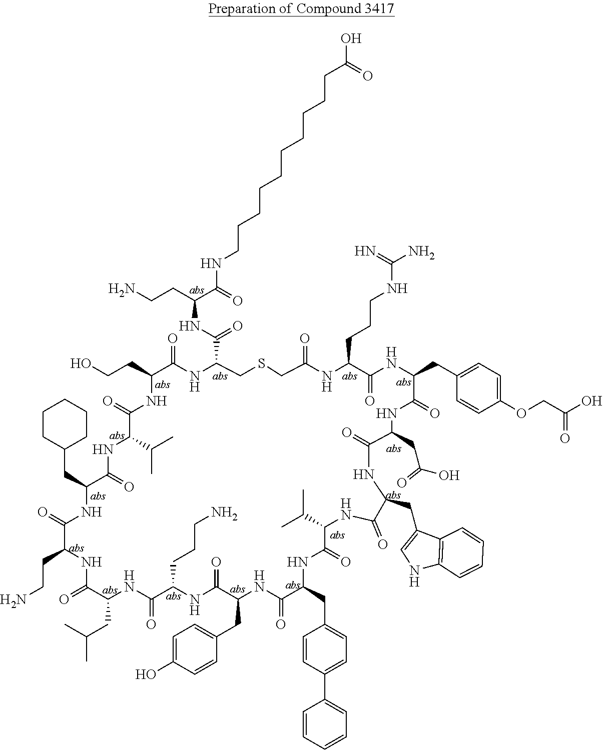 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC2CCCCC2)NC(=O)[C@H](CCN)NC1=O]

Compound 3417 was prepared on a 50 μmol scale. The yield of the product was 47.6 mg, and its estimated purity by LCMS analysis was 94.9%. Analysis condition A: Retention time=1.56, 1.6 min; ESI-MS(+) m/z [M+2H]²⁺: 1173.02, 1173.02.

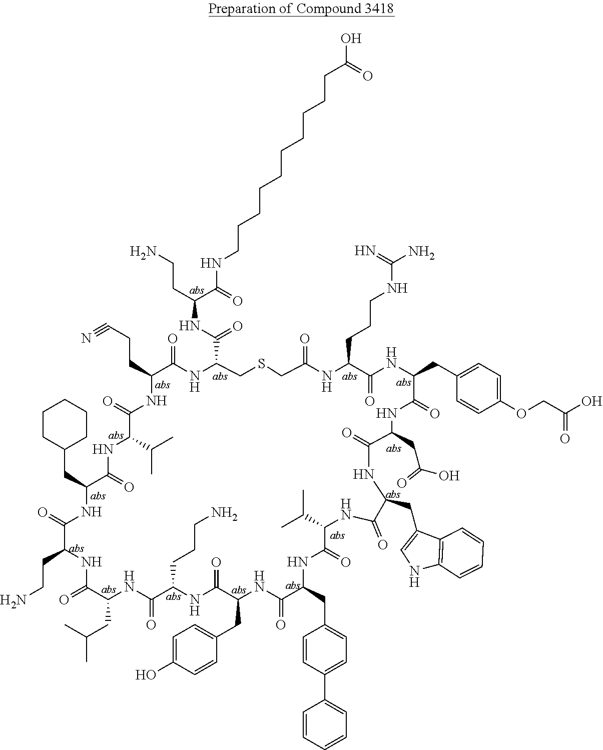 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC#N)NC(=O)[C@H](C(C)C)NC(=O)[C@H](CC2CCCCC2)NC(=O)[C@H](CCN)NC1=O]

Compound 3418 was prepared on a 50 μmol scale. The yield of the product was 78.6 mg, and its estimated purity by LCMS analysis was 96.8%. Analysis condition B: Retention time=1.54 min; ESI-MS(+) m/z [M+2H]²⁺: 1150.4.

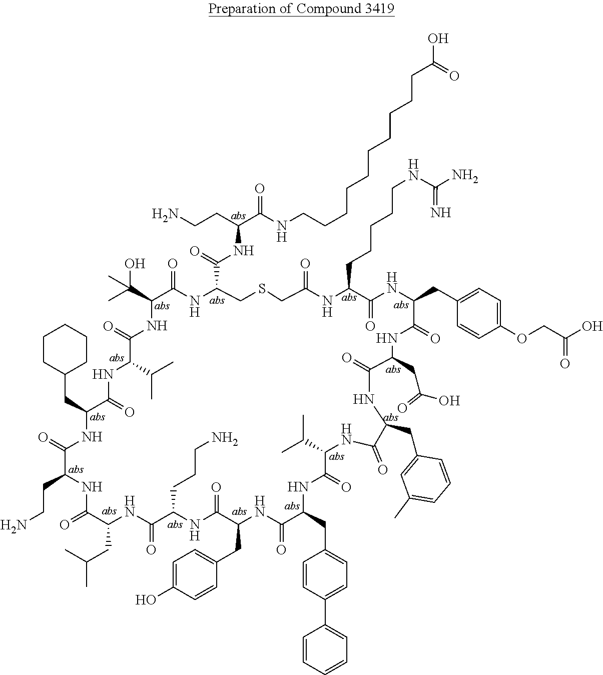 [SMILES: Cc1cccc(C[C@@H]2NC(=O)[C@H](CC(=O)O)NC(=O)[C@H](Cc3ccc(OCC(=O)O)cc3)NC(=O)[C@H](CCCCCNC(=N)N)NC(=O)CSC[C@@H](C(=O)N[C@@H](CCN)C(=O)NCCCCCCCCCCC(=O)O)NC(=O)[C@H](C(C)(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3419 was prepared on a 50 μmol scale. The yield of the product was 20.8 mg, and its estimated purity by LCMS analysis was 95.3%. Analysis condition B: Retention time=1.66 min; ESI-MS(+) m/z [M+2H]²⁺: 1139.9.

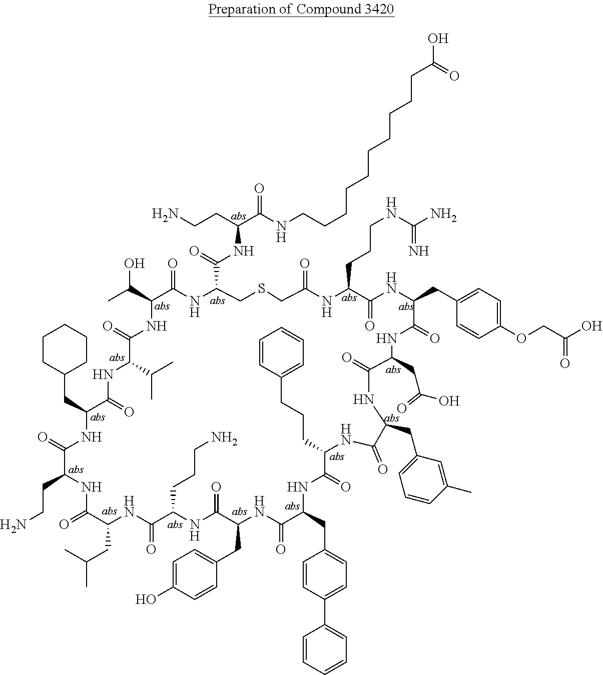 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CCCc3ccccc3)NC2=O)c1]

Compound 3420 was prepared on a 50 μmol scale. The yield of the product was 23.1 mg, and its estimated purity by LCMS analysis was 95.4%. Analysis condition B: Retention time=1.7 min; ESI-MS(+) m/z [M+3H]³⁺: 781.

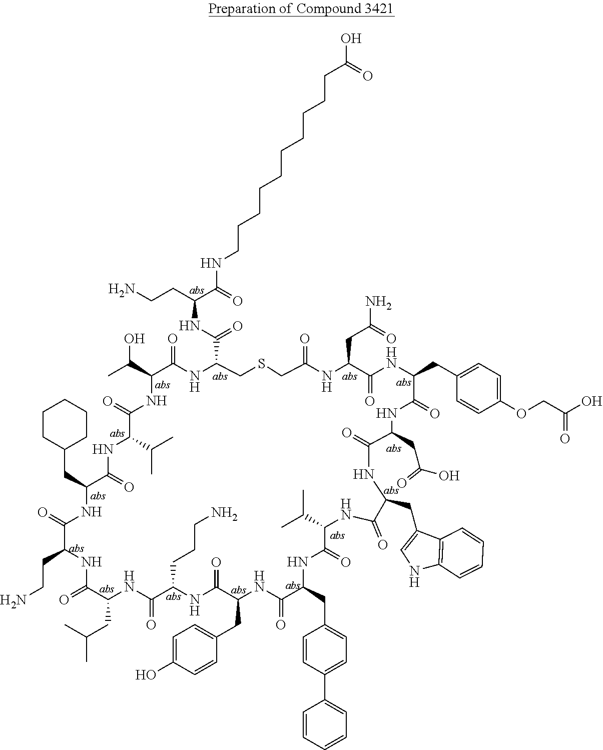 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CC(N)=O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3421 was prepared on a 50 μmol scale. The yield of the product was 17 mg, and its estimated purity by LCMS analysis was 97.3%. Analysis condition A: Retention time=1.59 min; ESI-MS(+) m/z [M+3H]³⁺: 750.1.

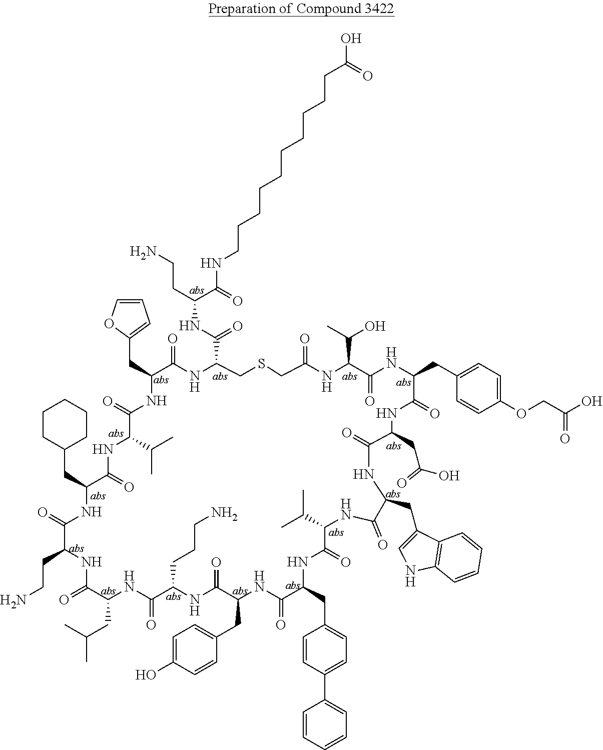 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CCN)C(=O)NCCCCCCCCCCC(=O)O)NC(=O)[C@H](Cc2ccco2)NC(=O)[C@H](C(C)C)NC(=O)[C@H](CC2CCCCC2)NC(=O)[C@H](CCN)NC1=O]

Compound 3422 was prepared on a 50 μmol scale. The yield of the product was 16 mg, and its estimated purity by LCMS analysis was 95.5%. Analysis condition A: Retention time=1.7 min; ESI-MS(+) m/z [M+2H]²⁺: 1136.1.

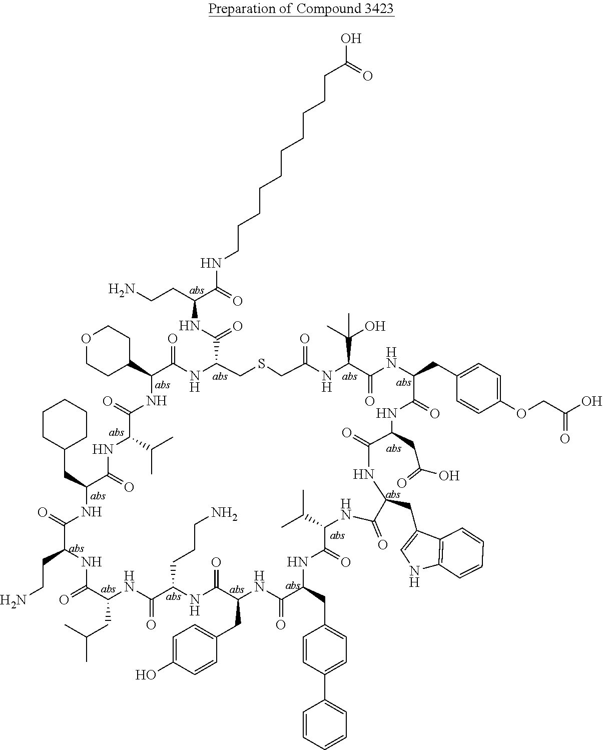 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)(C)O)NC(=O)CSC[C@@H](C(=O)N[C@@H](CCN)C(=O)NCCCCCCCCCCC(=O)O)NC(=O)[C@H](C2CCOCC2)NC(=O)[C@H](C(C)C)NC(=O)[C@H](CC2CCCCC2)NC(=O)[C@H](CCN)NC1=O]

Compound 3423 was prepared on a 50 μmol scale. The yield of the product was 18.5 mg, and its estimated purity by LCMS analysis was 94.9%. Analysis condition B: Retention time=1.65 min; ESI-MS(+) m/z [M+2H]²⁺: 1138.

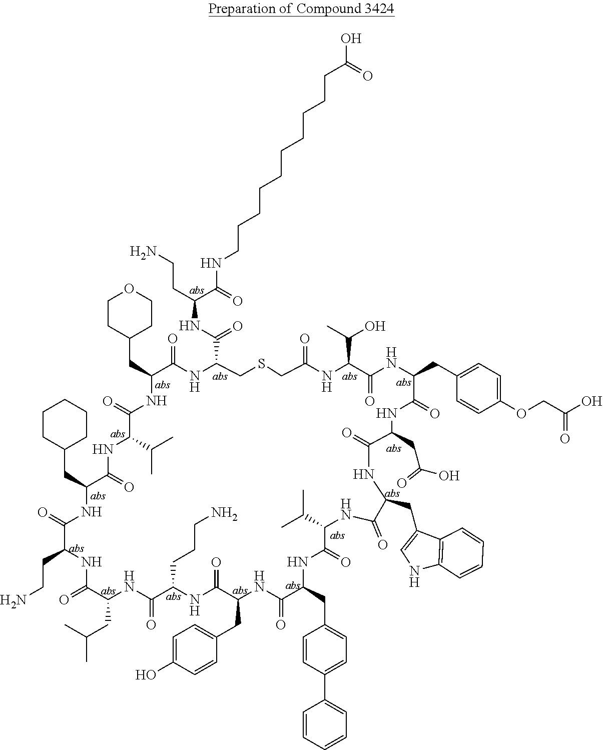 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CC2CCOCC2)NC(=O)[C@H](C(C)C)NC(=O)[C@H](CC2CCCCC2)NC(=O)[C@H](CCN)NC1=O]

Compound 3424 was prepared on a 50 μmol scale. The yield of the product was 49.4 mg, and its estimated purity by LCMS analysis was 97.2%. Analysis condition B: Retention time=1.68 min; ESI-MS(+) m/z [M+2H]²⁺: 1145.

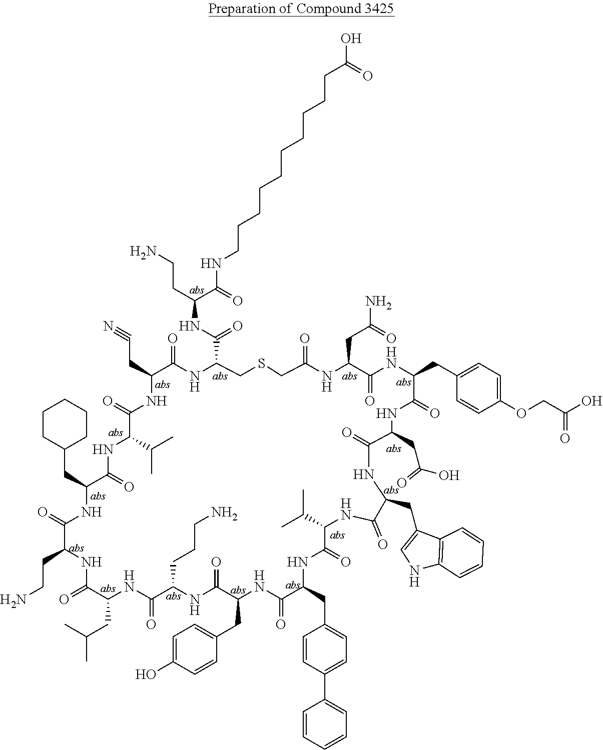 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CC(N)=O)NC(=O)CSC[C@@H](C(=O)N[C@@H](CCN)C(=O)NCCCCCCCCCCC(=O)O)NC(=O)[C@H](CC#N)NC(=O)[C@H](C(C)C)NC(=O)[C@H](CC2CCCCC2)NC(=O)[C@H](CCN)NC1=O]

Compound 3425 was prepared on a 50 μmol scale. The yield of the product was 57.3 mg, and its estimated purity by LCMS analysis was 87.5%. Analysis condition B: Retention time=1.63 min; ESI-MS(+) m/z [M+2H]²⁺: 1122.3.

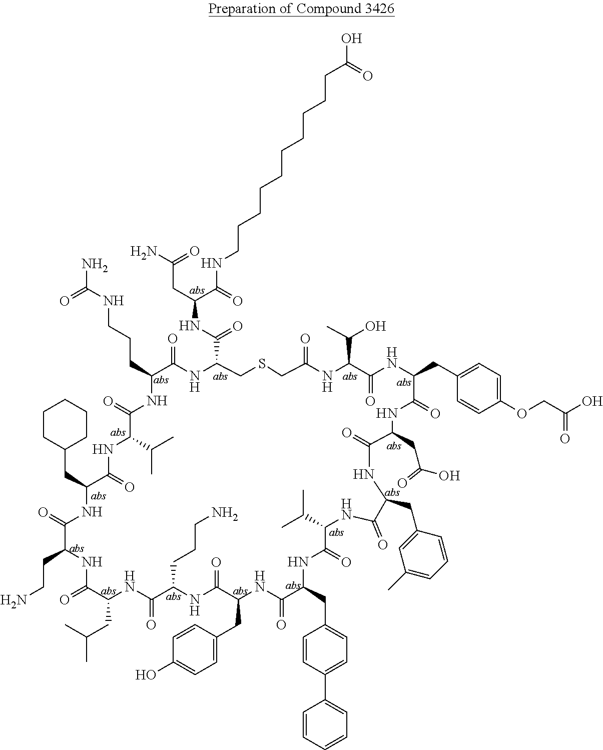 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CC(N)=O)C(=O)NCCCCCCCCCCC(=O)O)NC(=O)[C@H](CCCNC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3426 was prepared on a 50 μmol scale. The yield of the product was 34.3 mg, and its estimated purity by LCMS analysis was 95.1%. Analysis condition B: Retention time=1.76 min; ESI-MS(+) m/z [M+2H]²⁺: 1141.

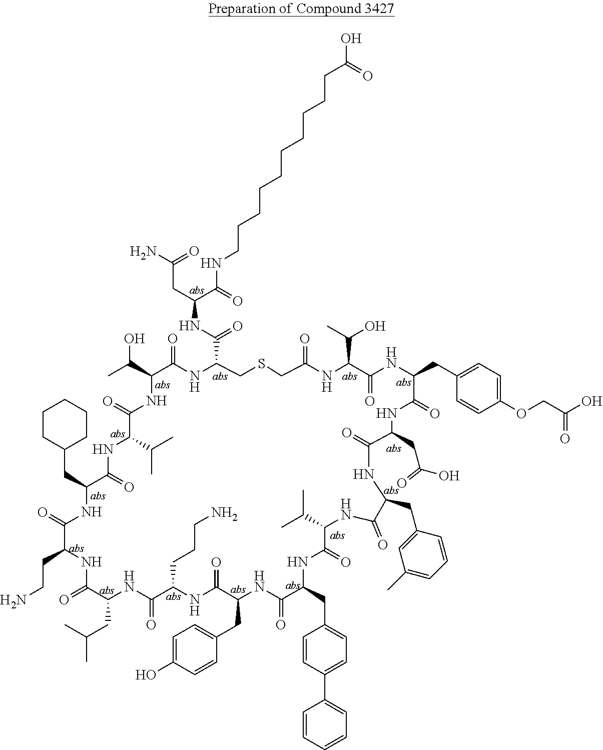 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3427 was prepared on a 50 μmol scale. The yield of the product was 33.6 mg, and its estimated purity by LCMS analysis was 90.2%. Analysis condition A: Retention time=1.74 min; ESI-MS(+) m/z [M+2H]²⁺: 1112.3.

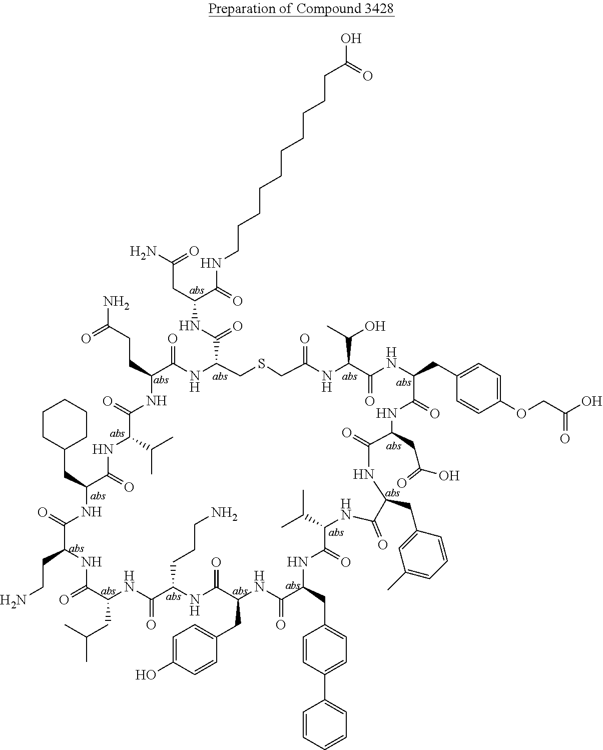 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3428 was prepared on a 50 μmol scale. The yield of the product was 30 mg, and its estimated purity by LCMS analysis was 98.2%. Analysis condition A: Retention time=1.74 min; ESI-MS(+) m/z [M+2H]²⁺: 1126.2.

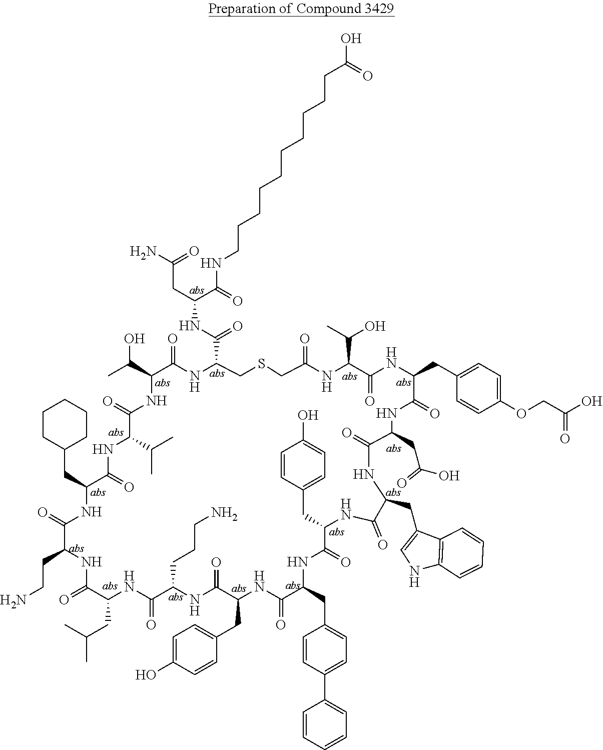 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3429 was prepared on a 50 μmol scale. The yield of the product was 56.4 mg, and its estimated purity by LCMS analysis was 95%. Analysis condition A: Retention time=1.59 min; ESI-MS(+) m/z [M+2H]²⁺:1157.

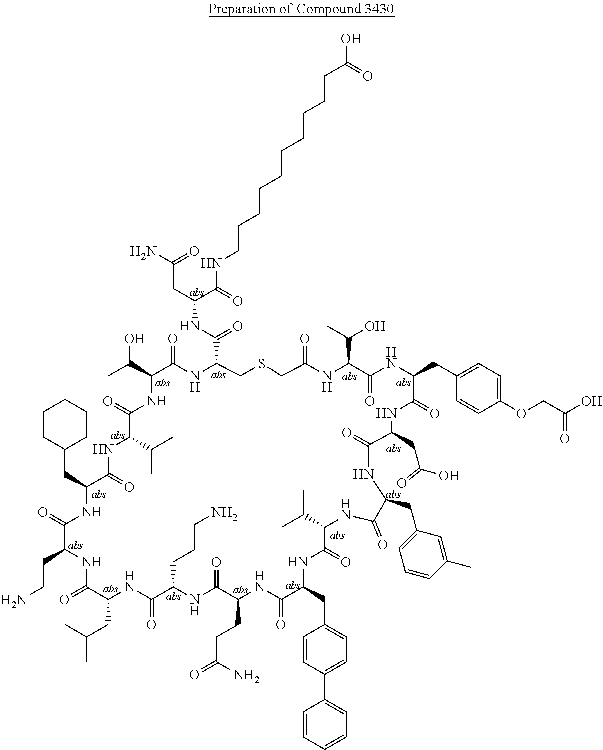 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](C(C)C)NC2=O)c1]

Compound 3430 was prepared on a 50 μmol scale. The yield of the product was 46.7 mg, and its estimated purity by LCMS analysis was 98.4%. Analysis condition B: Retention time=1.77 min; ESI-MS(+) m/z [M+2H]²⁺: 1095.1.

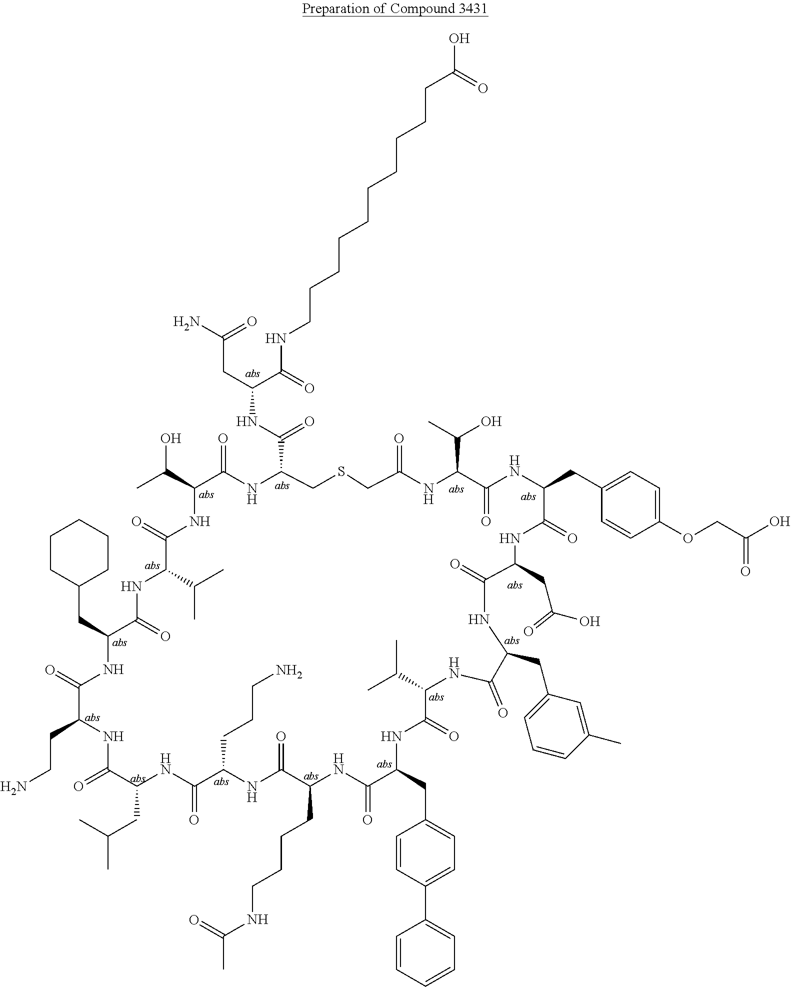 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3431 was prepared on a 50 μmol scale. The yield of the product was 31.1 mg, and its estimated purity by LCMS analysis was 95.7%. Analysis condition B: Retention time=1.8 min; ESI-MS(+) m/z [M+2H]²⁺: 1115.9.

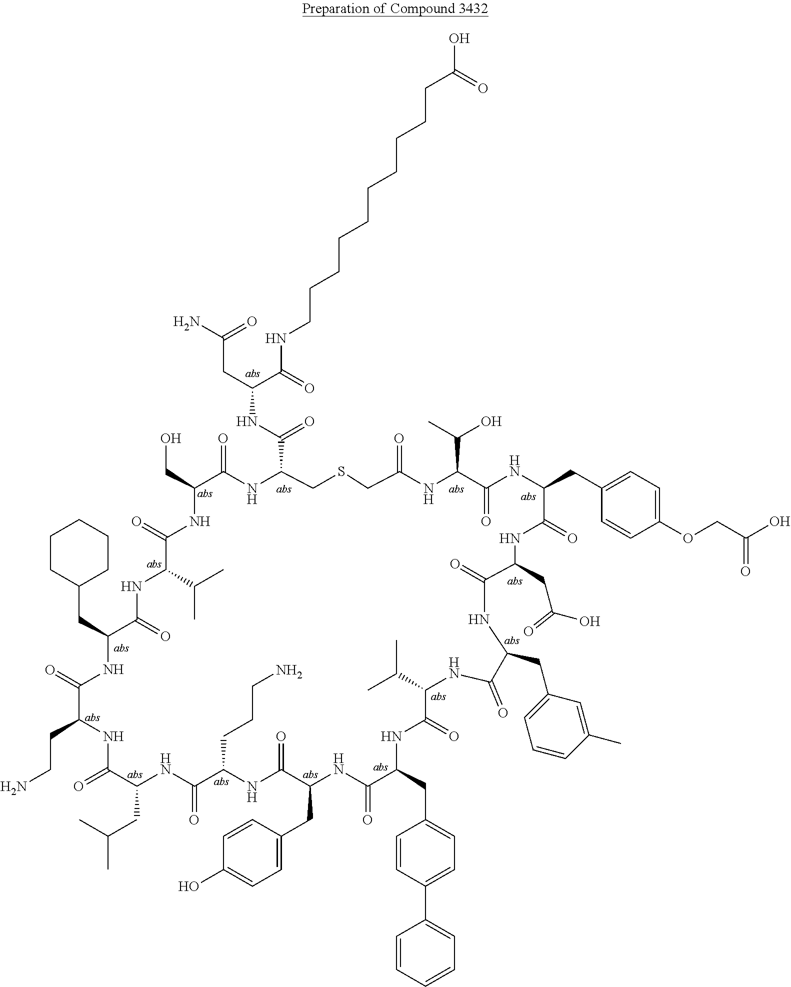 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3432 was prepared on a 50 μmol scale. The yield of the product was 11.5 mg, and its estimated purity by LCMS analysis was 99.2%. Analysis condition B: Retention time=1.66 min; ESI-MS(+) m/z [M+2H]²⁺: 1105.9.

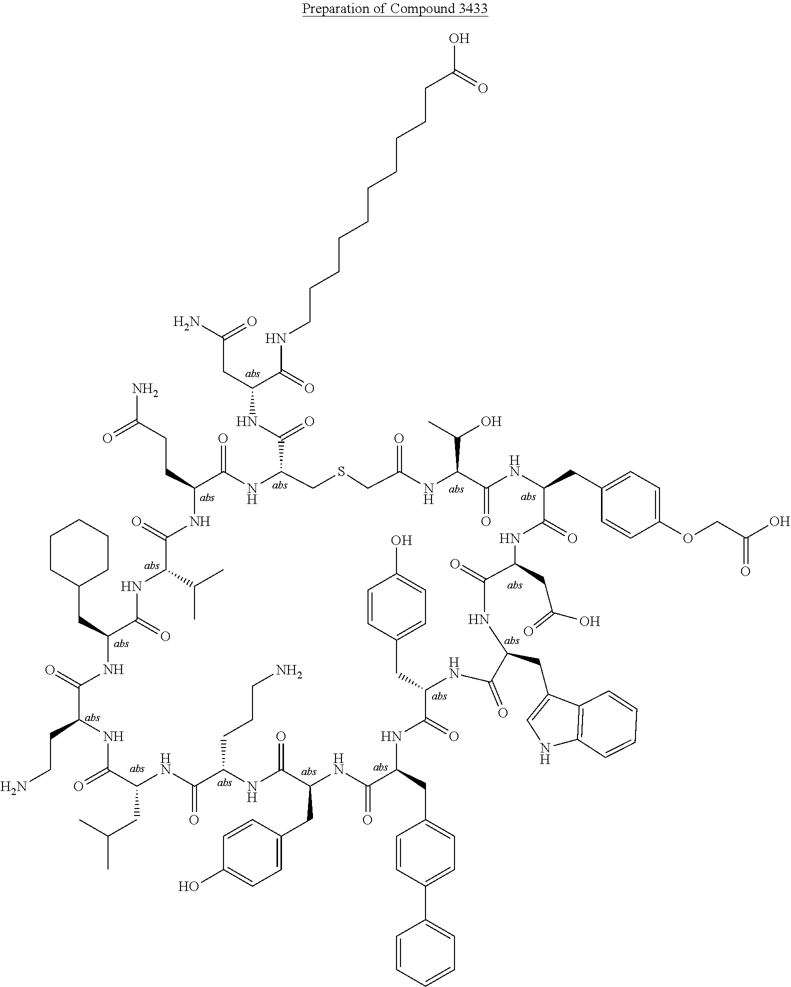 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC1=O]

Compound 3433 was prepared on a 50 μmol scale. The yield of the product was 57.3 mg, and its estimated purity by LCMS analysis was 92.8%. Analysis condition B: Retention time=1.69 min; ESI-MS(+) m/z [M+2H]²⁺: 1171.2.

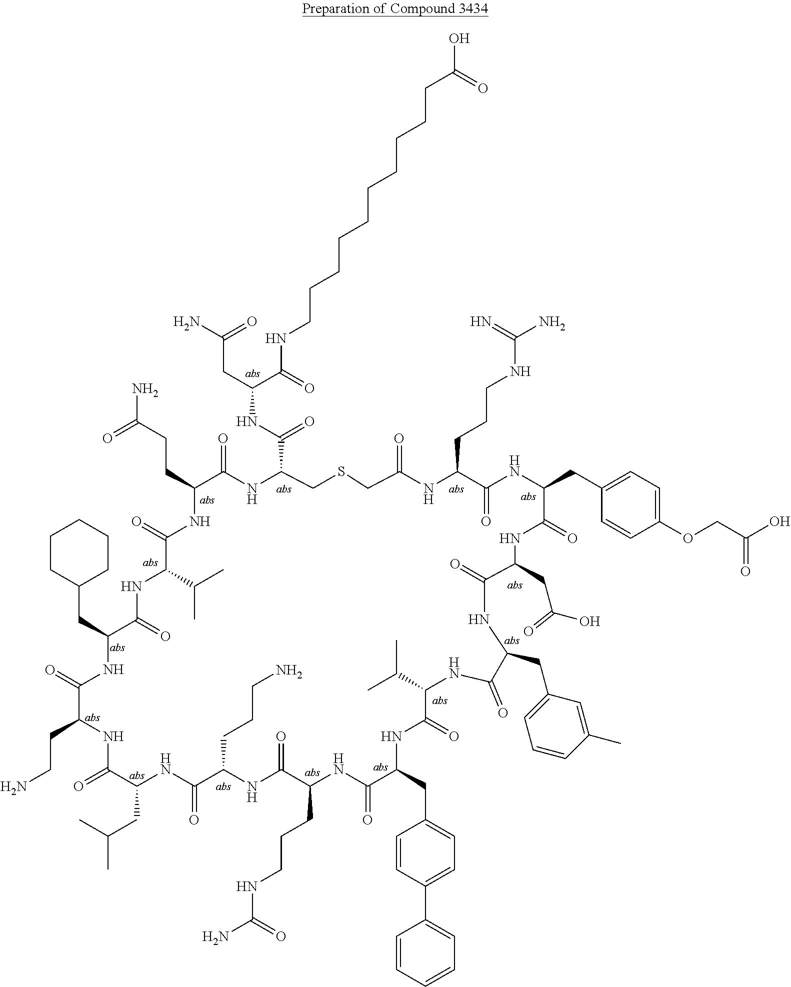 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3434 was prepared on a 50 μmol scale. The yield of the product was 24.2 mg, and its estimated purity by LCMS analysis was 92.2%. Analysis condition B: Retention time=1.65 min; ESI-MS(+) m/z [M+2H]²⁺: 1151.1.

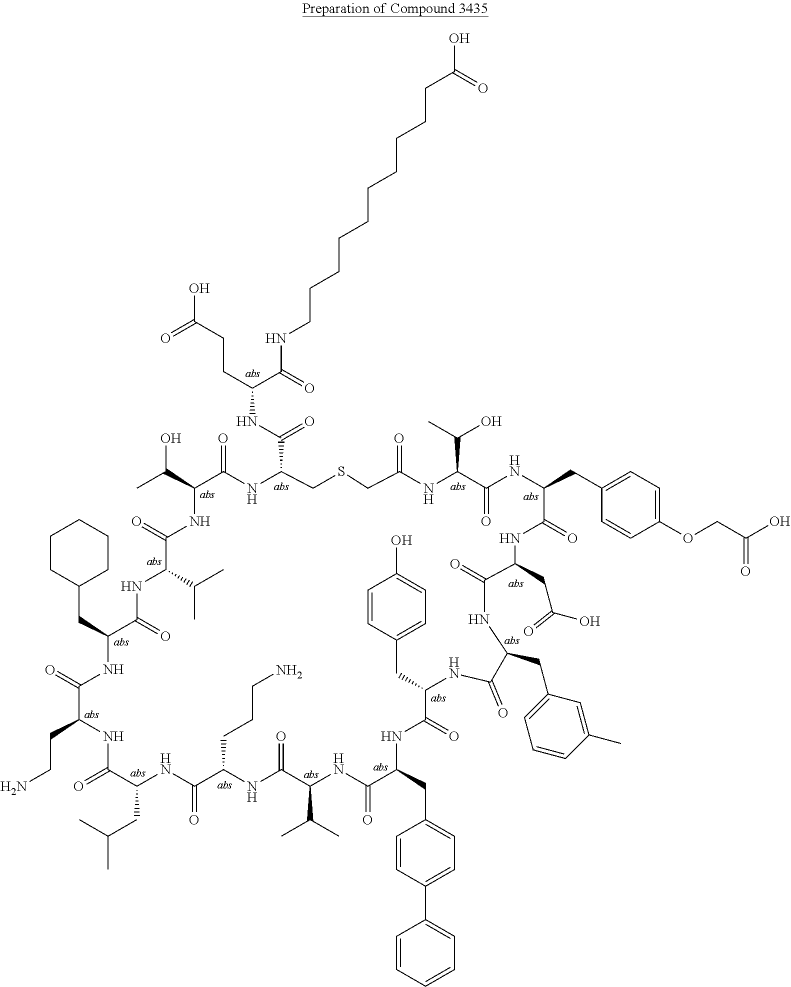 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CCC(=O)O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](Cc3ccc(O)cc3)NC2=O)c1]

Compound 3435 was prepared on a 50 μmol scale. The yield of the product was 63.9 mg, and its estimated purity by LCMS analysis was 100%. Analysis condition A: Retention time=1.64 min; ESI-MS(+) m/z [M+2H]²⁺: 1120.

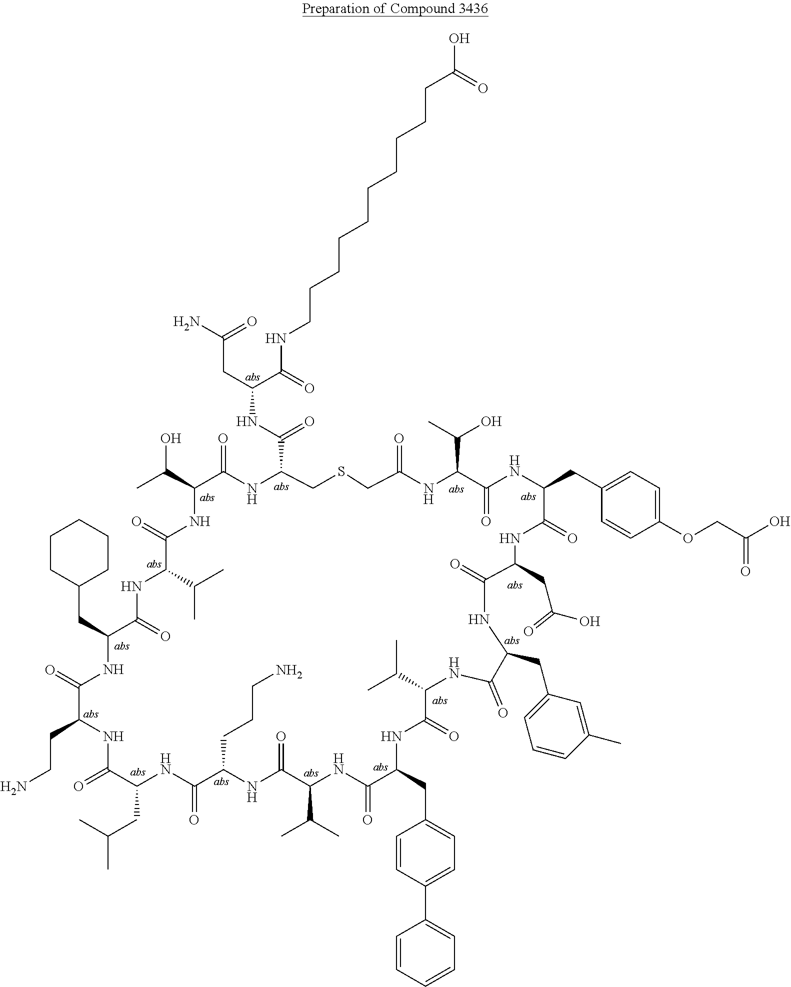 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3436 was prepared on a 50 μmol scale. The yield of the product was 10.4 mg, and its estimated purity by LCMS analysis was 100%. Analysis condition A: Retention time=1.74 min; ESI-MS(+) m/z [M+2H]²⁺: 1080.2.

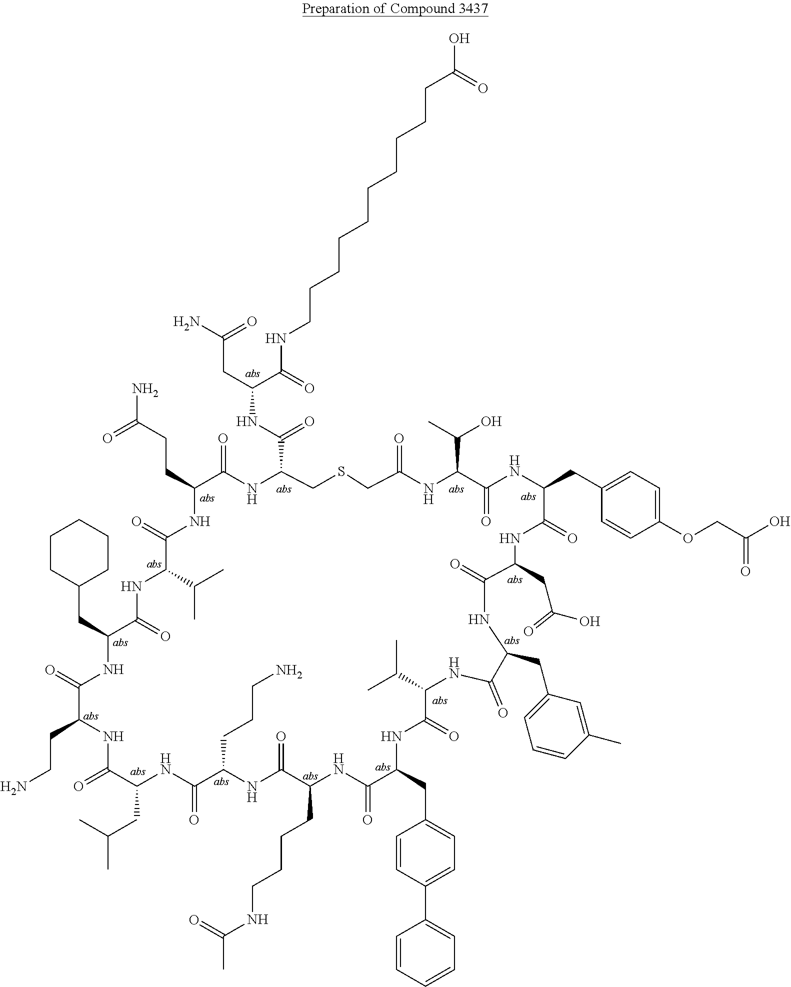 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(=O)NCCCCCCCCCCC(=O)O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3437 was prepared on a 50 μmol scale. The yield of the product was 12 mg, and its estimated purity by LCMS analysis was 100%. Analysis condition A: Retention time=1.65 min; ESI-MS(+) m/z [M+2H]²⁺: 1130.

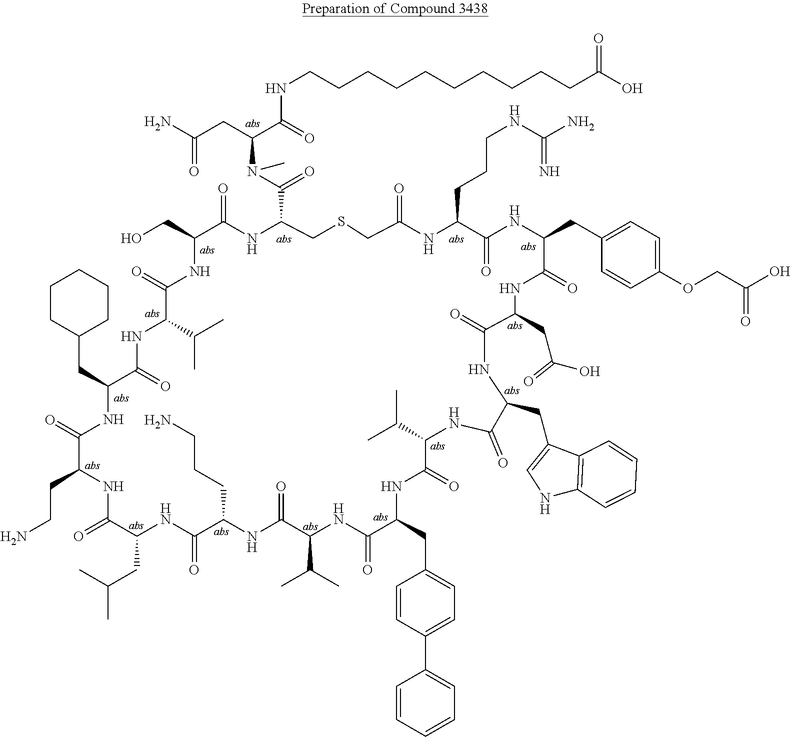 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N(C)[C@@H](CC(N)=O)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3438 was prepared on a 50 μmol scale. The yield of the product was 5.6 mg, and its estimated purity by LCMS analysis was 97.4%. Analysis condition B: Retention time=1.68 min; ESI-MS(+) m/z [M+3H]³⁺: 747.

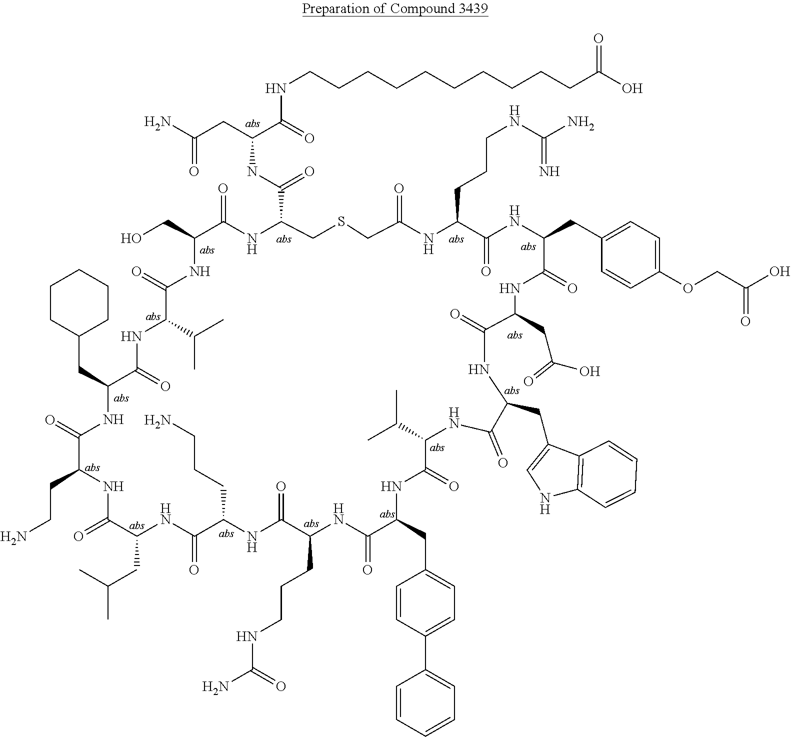 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CO)NC(=O)[C@H](C(C)C)NC(=O)[C@H](CC2CCCCC2)NC(=O)[C@H](CCN)NC1=O]

Compound 3439 was prepared on a 50 μmol scale. The yield of the product was 7.3 mg, and its estimated purity by LCMS analysis was 97.8%. Analysis condition B: Retention time=1.67 min; ESI-MS(+) m/z [M+3H]³⁺: 781.

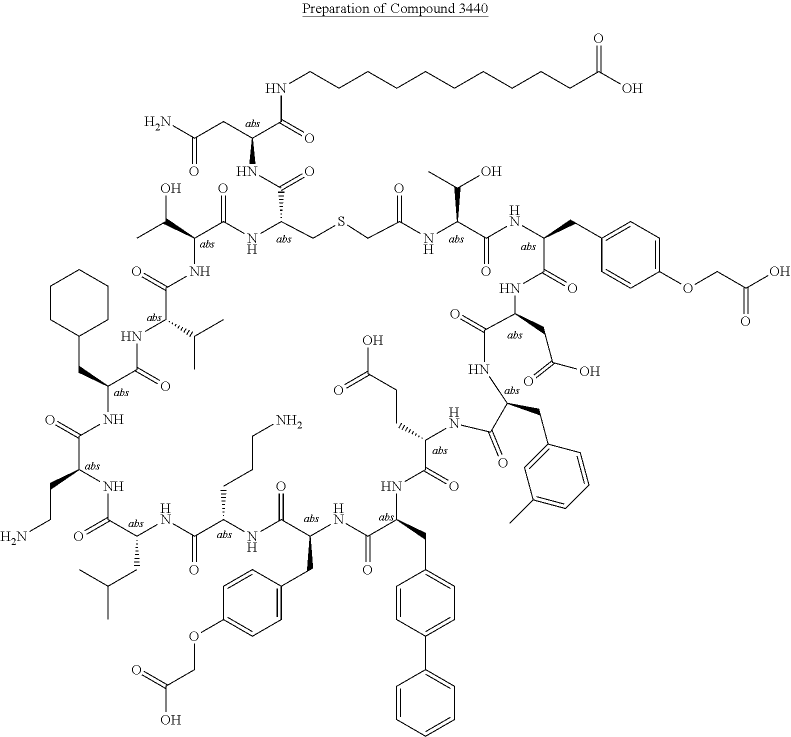 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](CCC(=O)O)NC2=O)c1]

Compound 3440 was prepared on a 50 μmol scale. The yield of the product was 5.9 mg, and its estimated purity by LCMS analysis was 95.8%. Analysis condition B: Retention time-1.73 min; ESI-MS(+) m/z [M+3H]³⁺: 767.

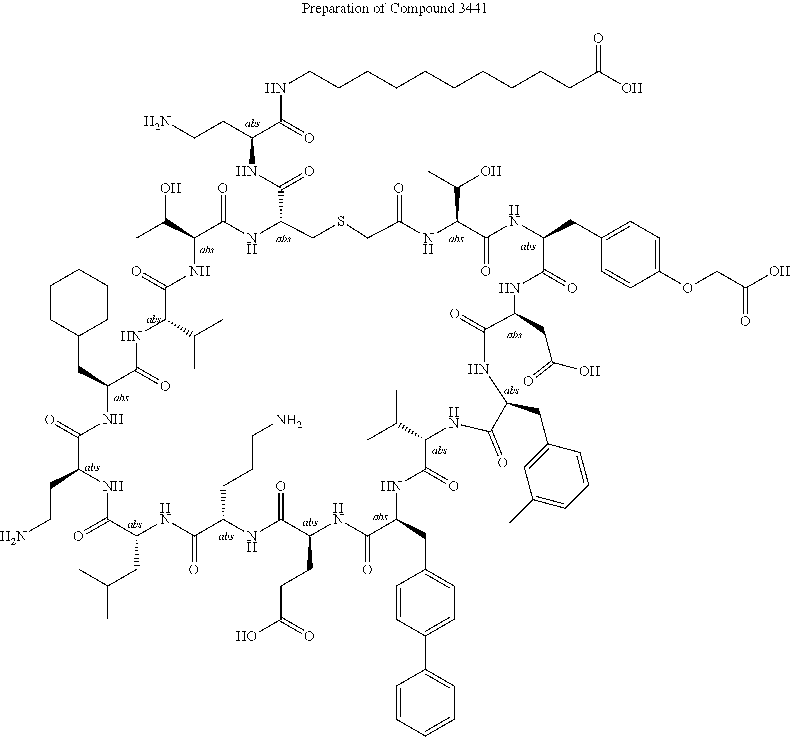 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 3441 was prepared on a 50 μmol scale. The yield of the product was 19.9 mg, and its estimated purity by LCMS analysis was 97.9%. Analysis condition B: Retention time-1.75 min; ESI-MS(+) m/z [M+3H]³⁺: 725.9.

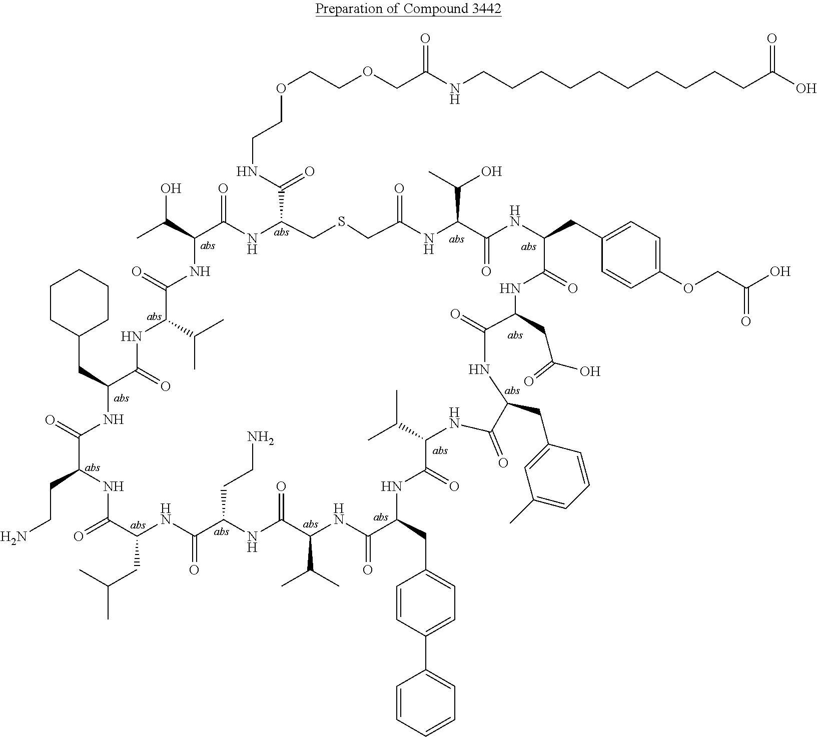 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](C(C)C)NC(=O)[C@H](Cc3ccc(-c4ccccc4)cc3)NC(=O)[C@H](C(C)C)NC2=O)c1]

Compound 3442 was prepared on a 50 μmol scale. The yield of the product was 17.2 mg, and its estimated purity by LCMS analysis was 97.4%. Analysis condition B: Retention time=1.9 min; ESI-MS(+) m/z [M+2H]²⁺: 1089.

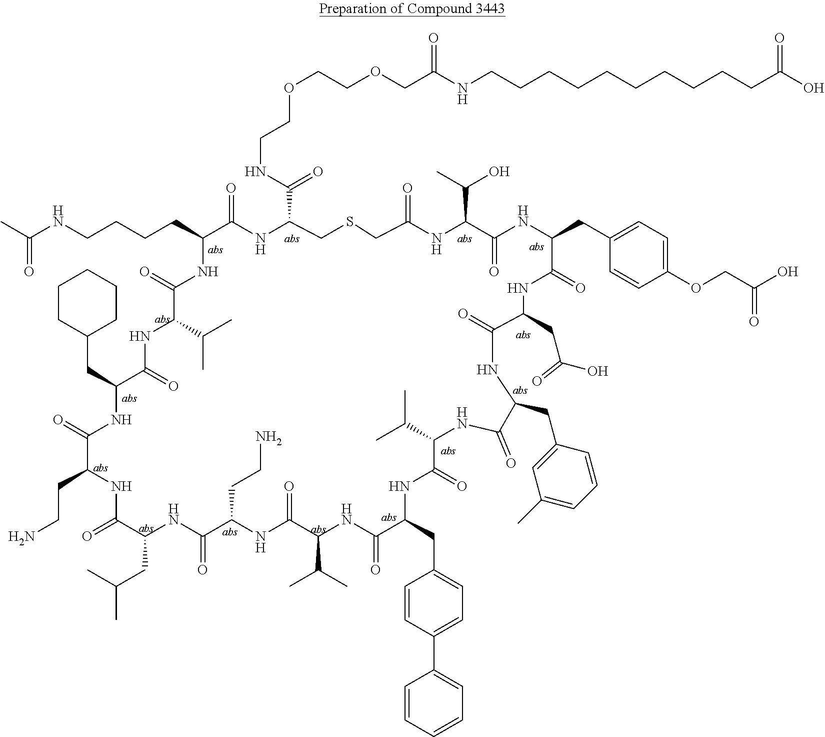 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC1=O]

Compound 3443 was prepared on a 50 μmol scale. The yield of the product was 19.6 mg, and its estimated purity by LCMS analysis was 95.6%. Analysis condition A: Retention time=1.82 min; ESI-MS(+) m/z [M+2H]²⁺: 1123.2.

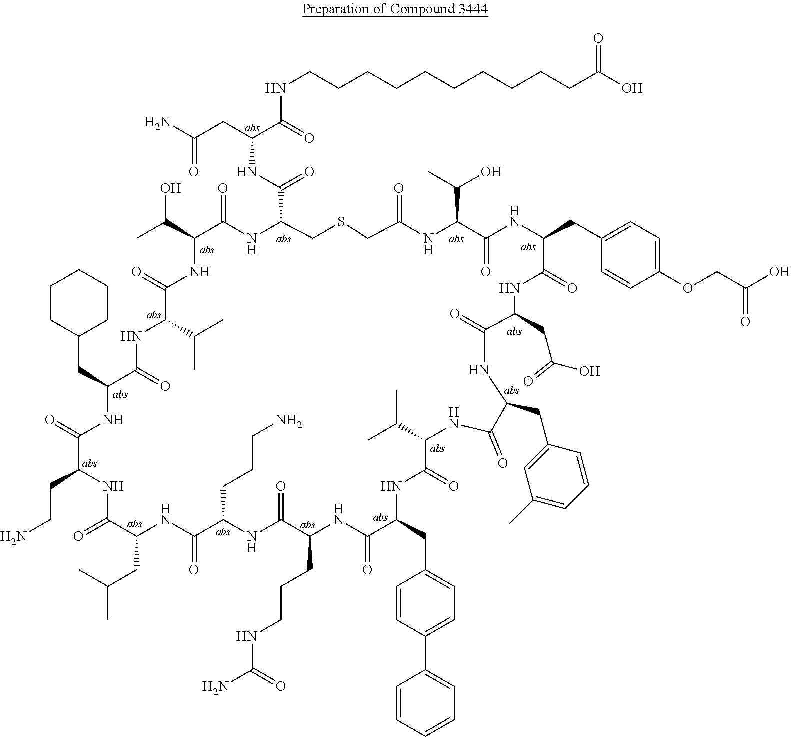 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3444 was prepared on a 50 μmol scale. The yield of the product was 36.9 mg, and its estimated purity by LCMS analysis was 96.1%. Analysis condition B: Retention time=1.85 min; ESI-MS(+) m/z [M+3H]³⁺: 740.2.

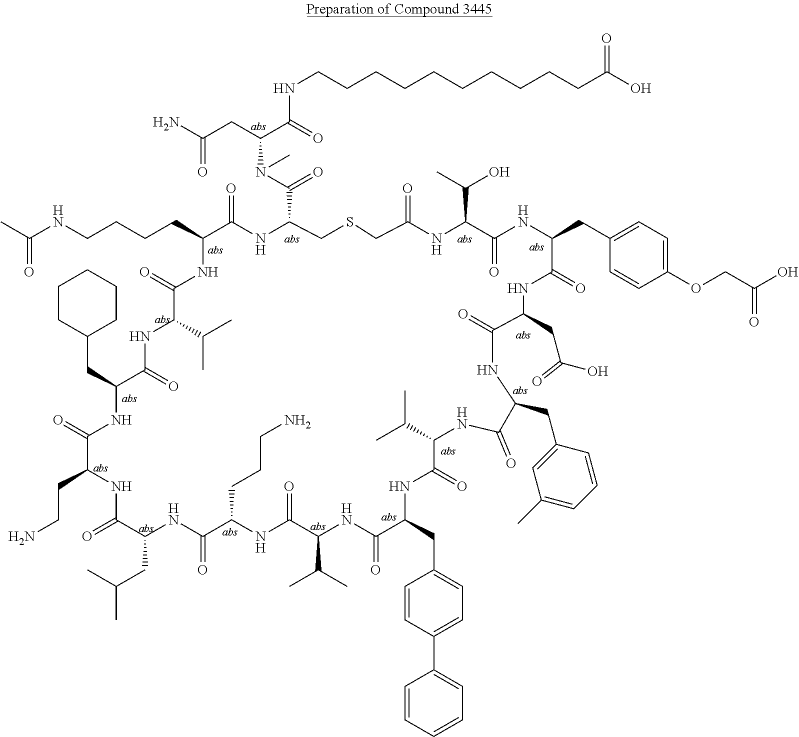 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N(C)[C@H](CC(N)=O)C(=O)NCCCCCCCCCCC(=O)O)NC1=O]

Compound 3445 was prepared on a 50 μmol scale. The yield of the product was 2 mg, and its estimated purity by LCMS analysis was 100%. Analysis condition A: Retention time=1.72 min; ESI-MS(+) m/z [M+2H]²⁺: 1122.1.

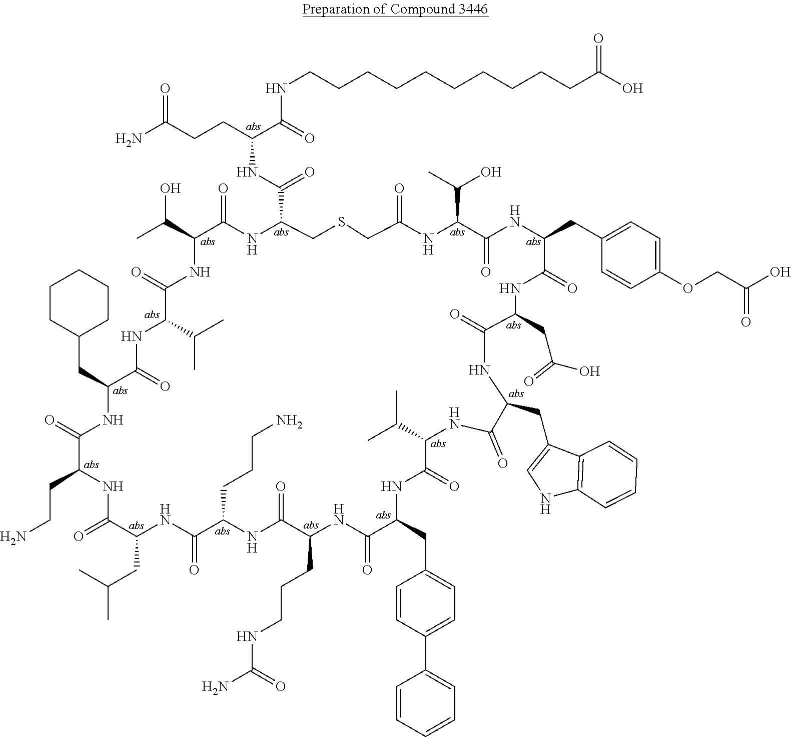 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CCC(N)=O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3446 was prepared on a 50 μmol scale. The yield of the product was 9 mg, and its estimated purity by LCMS analysis was 88.1%. Analysis condition B: Retention time=1.66 min; ESI-MS(+) m/z [M+2H]²⁺: 1129.

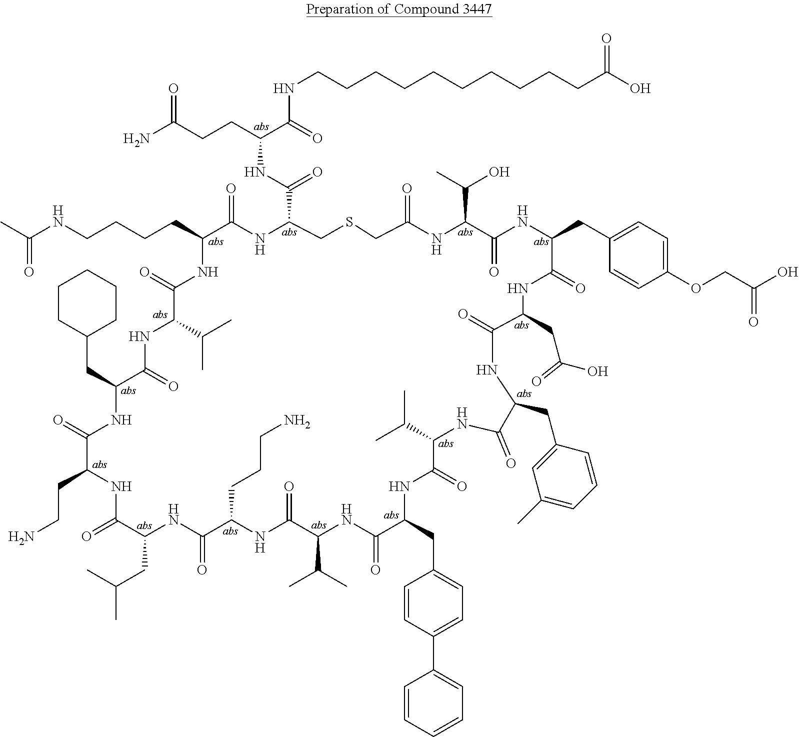 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CCC(N)=O)C(=O)NCCCCCCCCCCC(=O)O)NC1=O]

Compound 3447 was prepared on a 50 μmol scale. The yield of the product was 19.3 mg, and its estimated purity by LCMS analysis was 96.2%. Analysis condition A: Retention time=1.69 min; ESI-MS(+) m/z [M+2H]²⁺: 1122.

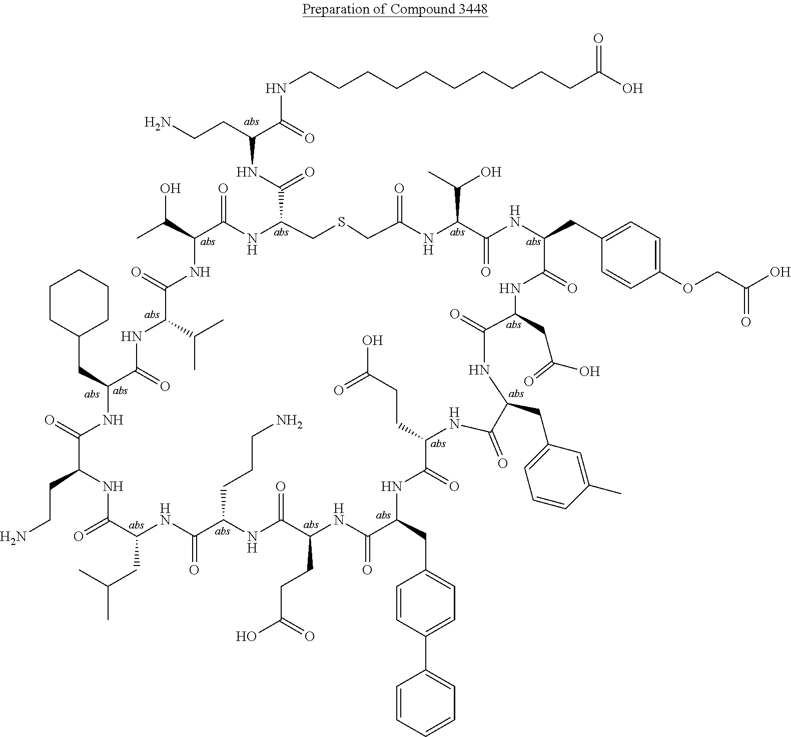

Compound 3448 was prepared on a 50 μmol scale. The yield of the product was 17.6 mg, and its estimated purity by LCMS analysis was 90.1%. Analysis condition A: Retention time=1.53 min; ESI-MS(+) m/z [M+3H]³⁺: 736.1.

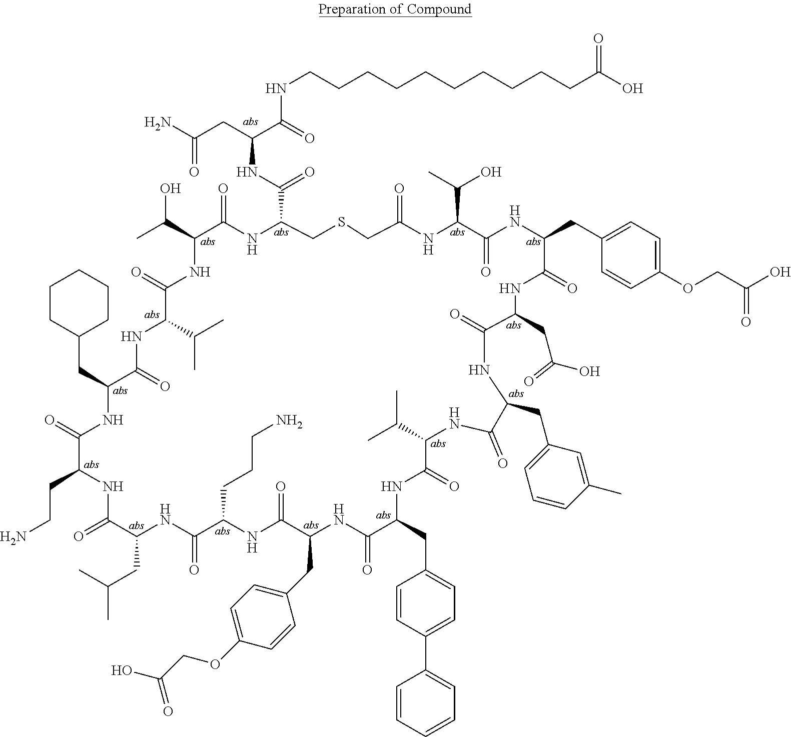 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 3449 was prepared on a 50 μmol scale. The yield of the product was 18 mg, and its estimated purity by LCMS analysis was 97.8%. Analysis condition B: Retention time=1.76 min; ESI-MS(+) m/z [M+2H]²⁺: 1134.4.

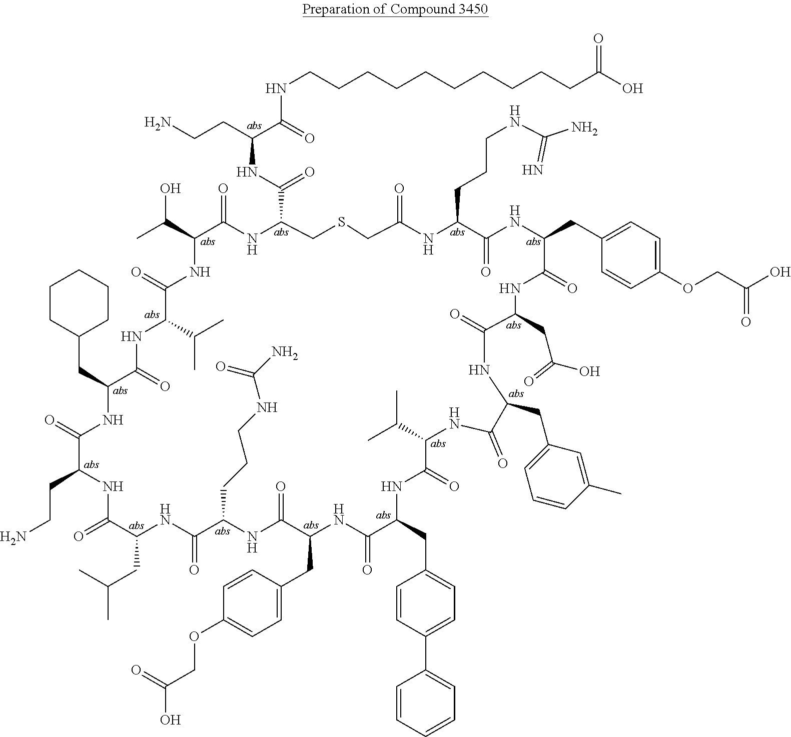 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 3450 was prepared on a 50 μmol scale. The yield of the product was 15.6 mg, and its estimated purity by LCMS analysis was 100%. Analysis condition A: Retention time=1.57 min; ESI-MS(+) m/z [M+3H]³⁺: 789.1.

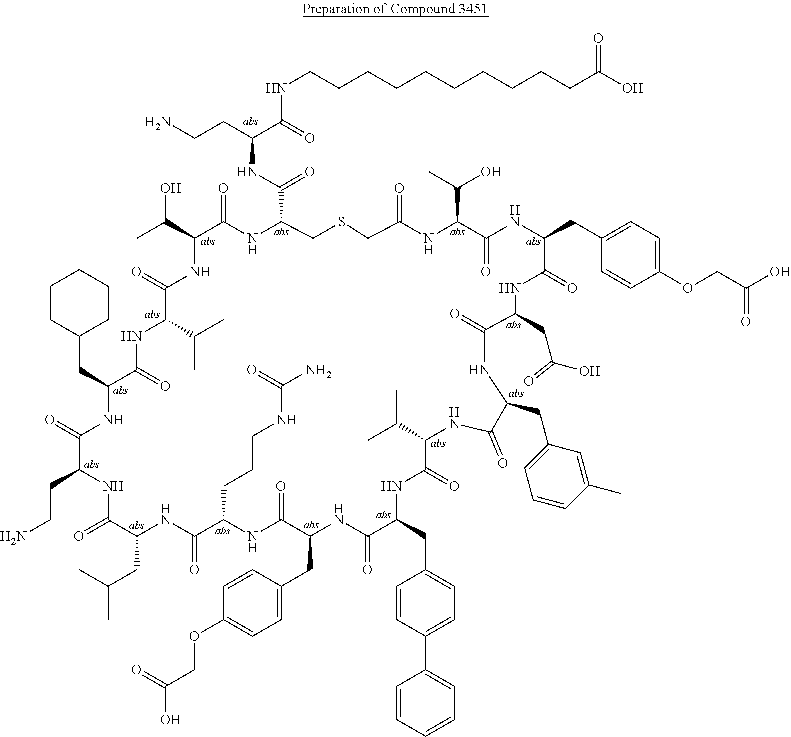 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 3451 was prepared on a 50 μmol scale. The yield of the product was 16.2 mg, and its estimated purity by LCMS analysis was 96.7%. Analysis condition B: Retention time=1.88 min; ESI-MS(+) m/z [M+2H]²⁺: 1156.2.

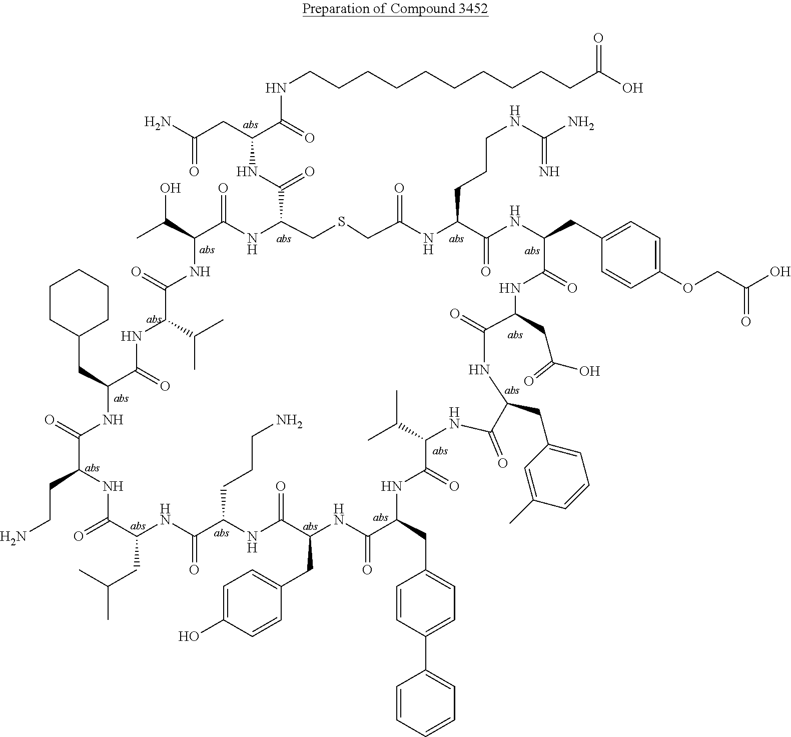 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3452 was prepared on a 50 μmol scale. The yield of the product was 44.1 mg, and its estimated purity by LCMS analysis was 96%. Analysis condition A: Retention time=1.68 min; ESI-MS(+) m/z [M+2H]²⁺: 1139.9.

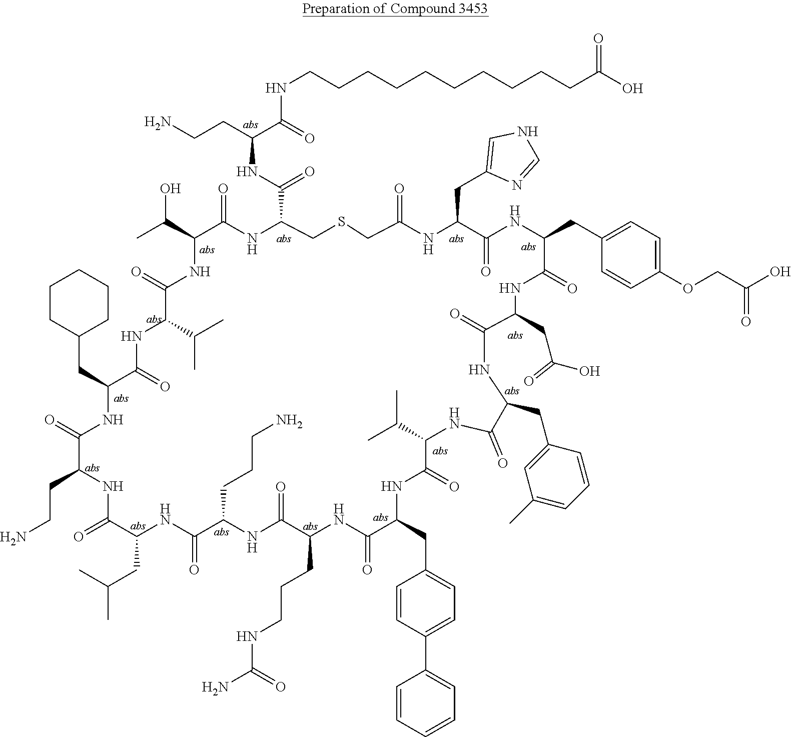 [SMILES: Cc1cccc(C[C@@H]2NC(=O)[C@H](CC(=O)O)NC(=O)[C@H](Cc3ccc(OCC(=O)O)cc3)NC(=O)[C@H](Cc3c[nH]cn3)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3453 was prepared on a 50 μmol scale. The yield of the product was 5.3 mg, and its estimated purity by LCMS analysis was 100%. Analysis condition A: Retention time=1.59 min; ESI-MS(+) m/z [M+3H]³⁺: 747.1.

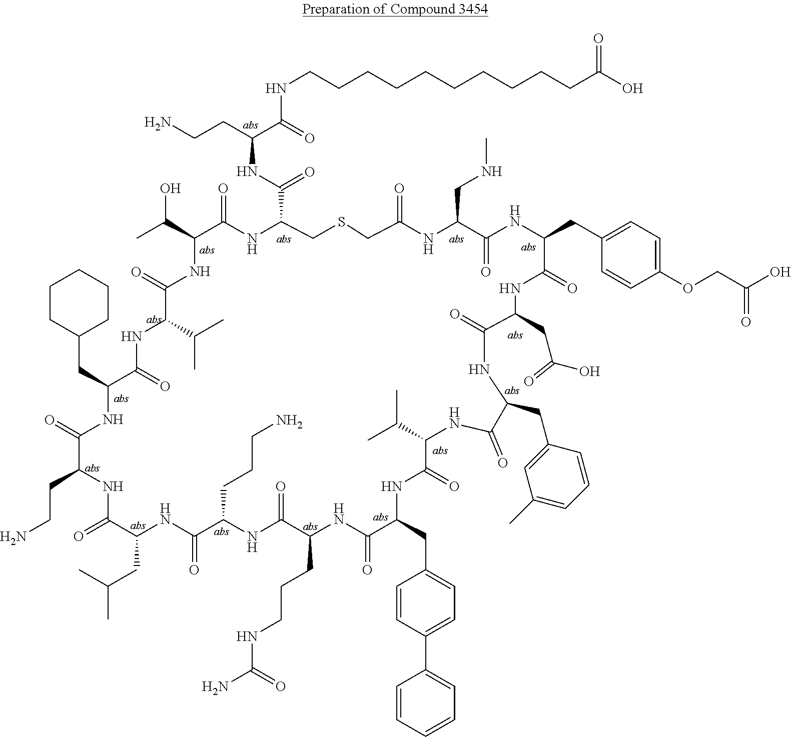 [SMILES: CNC[C@@H]1NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC1=O]

Compound 3454 was prepared on a 50 μmol scale. The yield of the product was 12.6 mg, and its estimated purity by LCMS analysis was 100%. Analysis condition A: Retention time=1.68 min; ESI-MS(+) m/z [M+2H]²⁺: 1102.1.

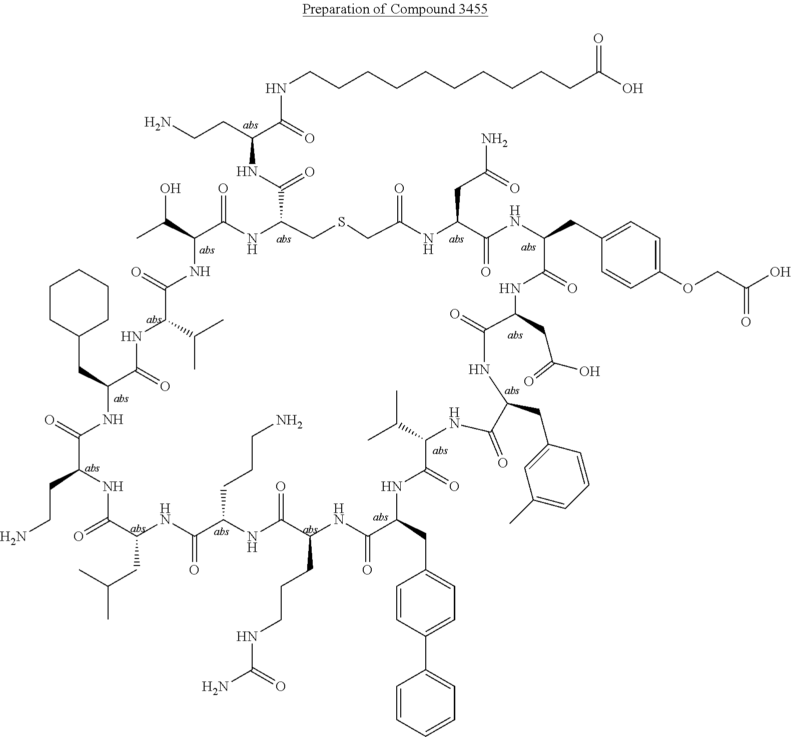 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3455 was prepared on a 50 μmol scale. The yield of the product was 17.6 mg, and its estimated purity by LCMS analysis was 100%. Analysis condition A: Retention time=1.69 min; ESI-MS(+) m/z [M+2H]²⁺: 1108.9.

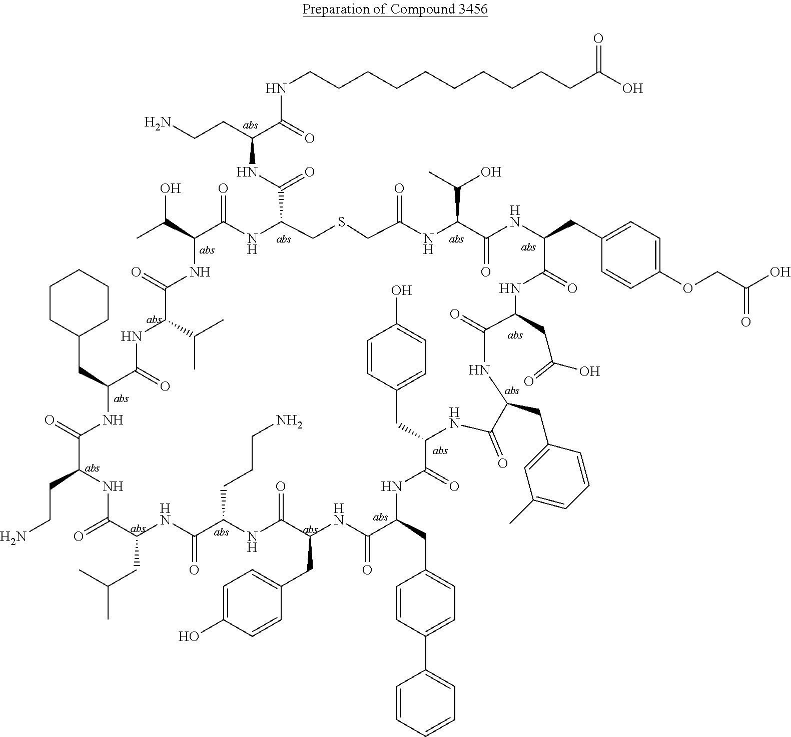 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 3456 was prepared on a 50 μmol scale. The yield of the product was 27.3 mg, and its estimated purity by LCMS analysis was 96.2%. Analysis condition A: Retention time-1.66 min; ESI-MS(+) m/z [M+2H]²⁺: 1137.9.

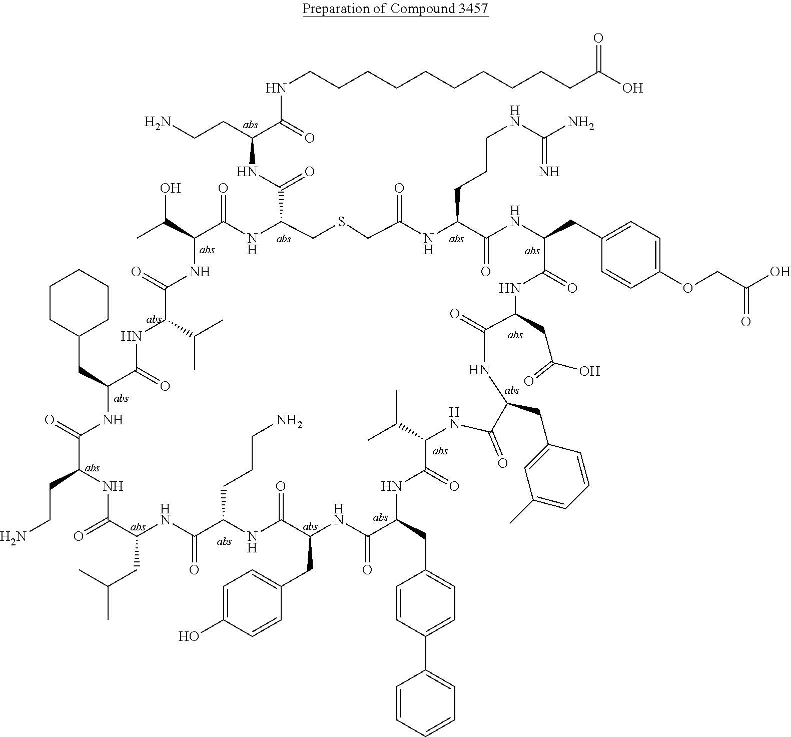 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3457 was prepared on a 50 μmol scale. The yield of the product was 15.4 mg, and its estimated purity by LCMS analysis was 95.6%. Analysis condition A: Retention time=1.58 min; ESI-MS(+) m/z [M+2H]²⁺: 1133.1.

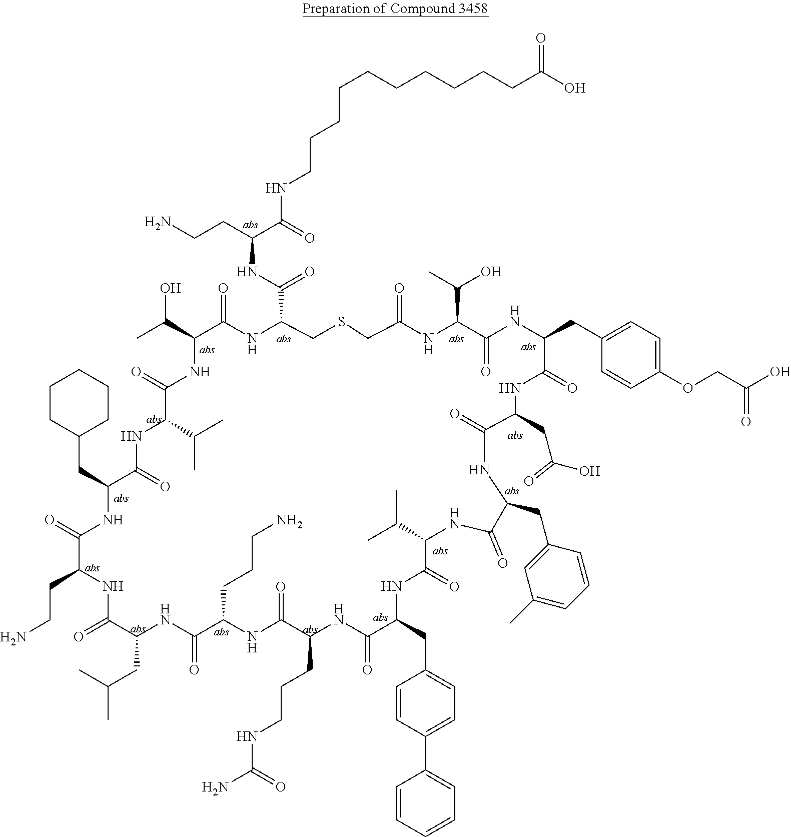 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3458 was prepared on a 50 μmol scale. The yield of the product was 6.1 mg, and its estimated purity by LCMS analysis was 98.6%. Analysis condition B: Retention time=1.78 min; ESI-MS(+) m/z [M+3H]³⁺: 735.1.

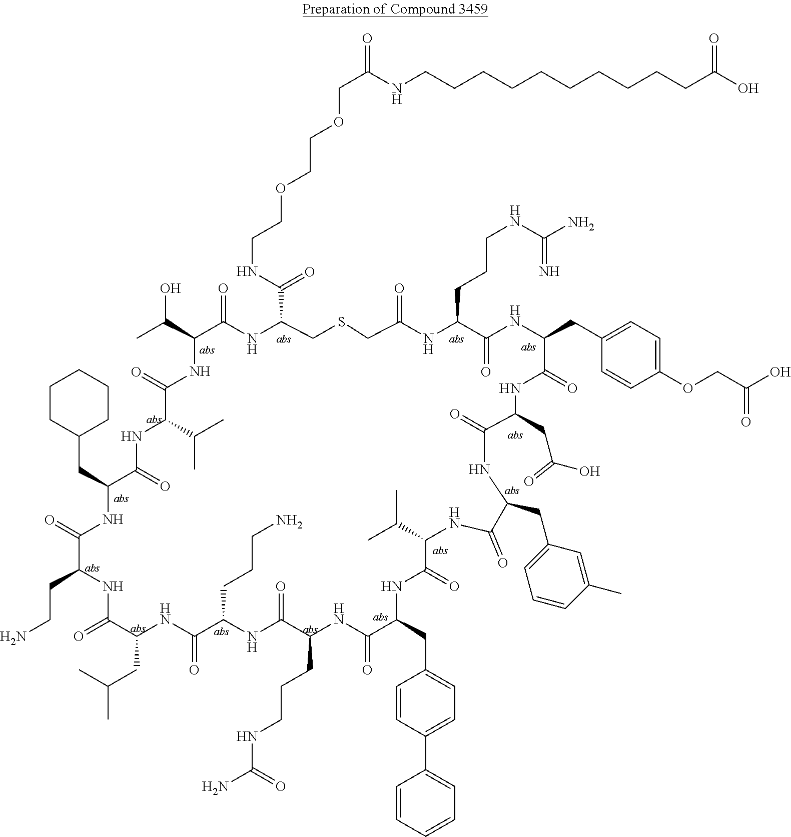 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3459 was prepared on a 50 μmol scale. The yield of the product was 49.5 mg, and its estimated purity by LCMS analysis was 95.9%. Analysis condition A: Retention time=1.67 min; ESI-MS(+) m/z [M+3H]³⁺: 768.7.

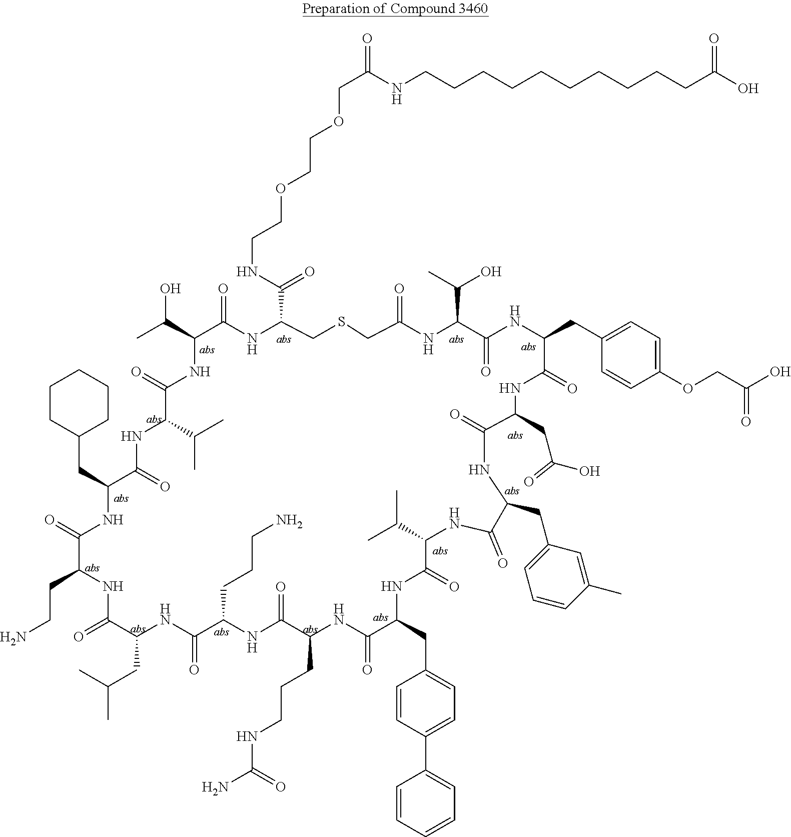 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3460 was prepared on a 50 μmol scale. The yield of the product was 17.8 mg, and its estimated purity by LCMS analysis was 95.1%. Analysis condition B: Retention time=1.75 min; ESI-MS(+) m/z [M+2H]²⁺: 1125.4.

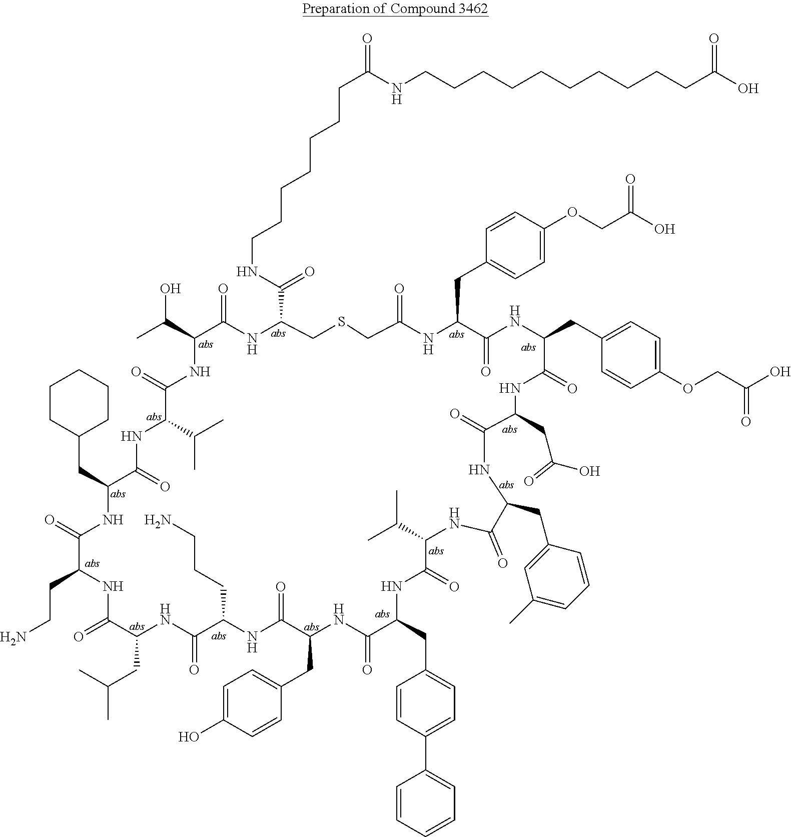 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCCCCCCCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3462 was prepared on a 50 μmol scale. The yield of the product was 38 mg, and its estimated purity by LCMS analysis was 96.1%. Analysis condition B: Retention time=1.94 min; ESI-MS(+) m/z [M+2H]²⁺: 1173.2.

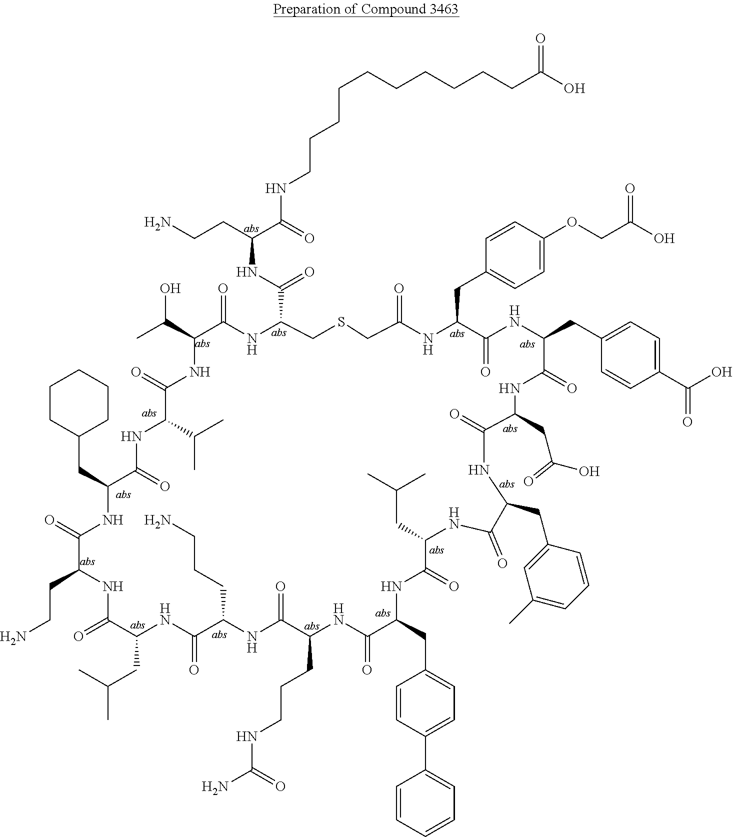 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](CC(C)C)NC2=O)c1]

Compound 3463 was prepared on a 50 μmol scale. The yield of the product was 18 mg, and its estimated purity by LCMS analysis was 97.2%. Analysis condition A: Retention time=1.74 min; ESI-MS(+) m/z [M+3H]³⁺: 770.

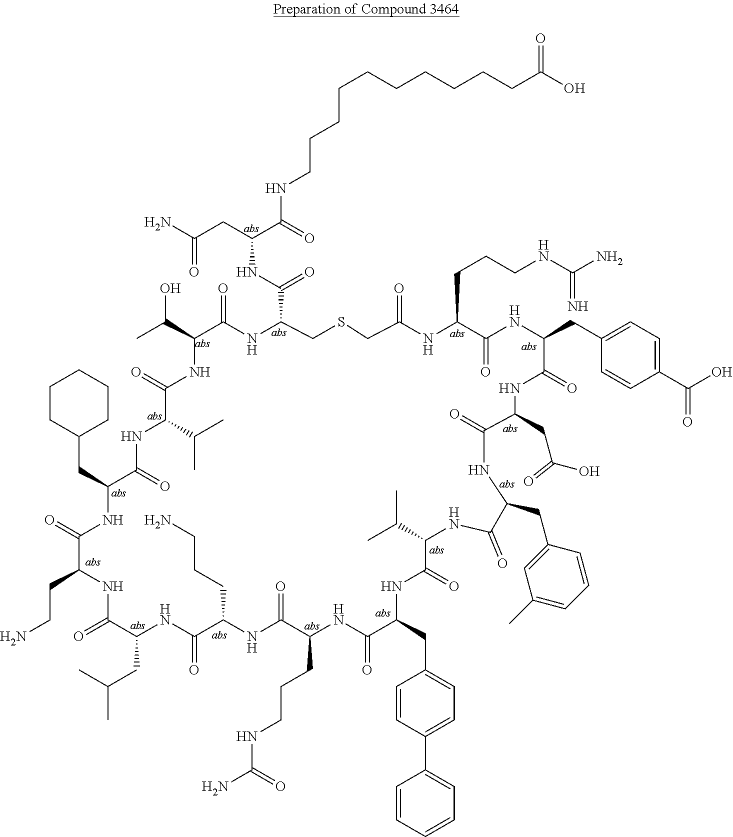 [SMILES: Cc1cccc(C[C@@H]2NC(=O)[C@H](CC(=O)O)NC(=O)[C@H](Cc3ccc(C(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3464 was prepared on a 50 μmol scale. The yield of the product was 25.1 mg, and its estimated purity by LCMS analysis was 96.2%. Analysis condition B: Retention time=1.76 min; ESI-MS(+) m/z [M+2H]²⁺: 1137.1.

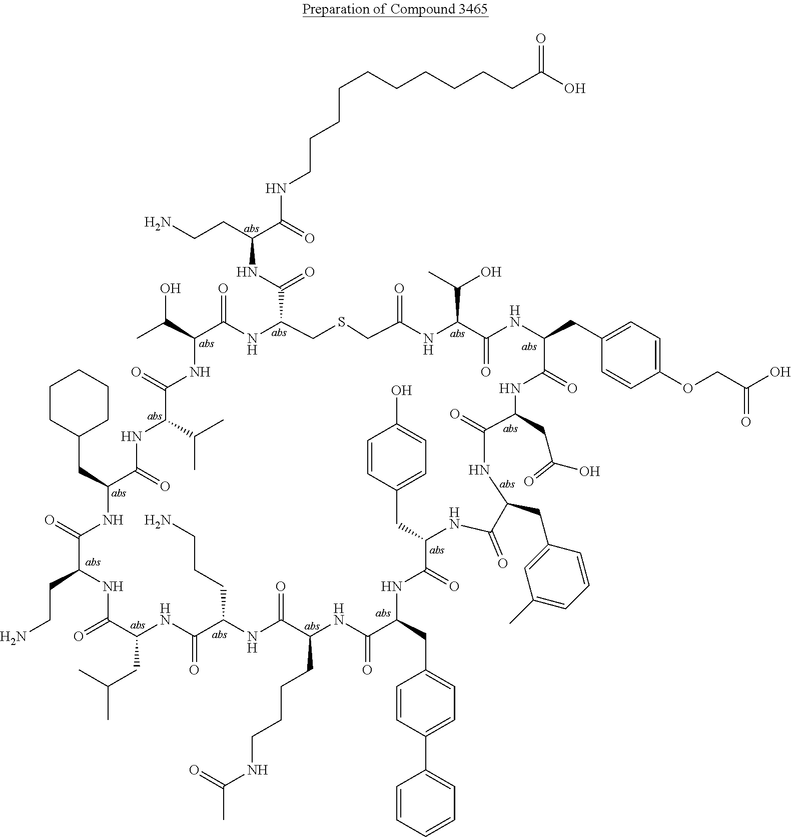 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3465 was prepared on a 50 μmol scale. The yield of the product was 17.7 mg, and its estimated purity by LCMS analysis was 92.5%. Analysis condition A: Retention time=1.63 min; ESI-MS(+) m/z [M+2H]²⁺: 1162.1.

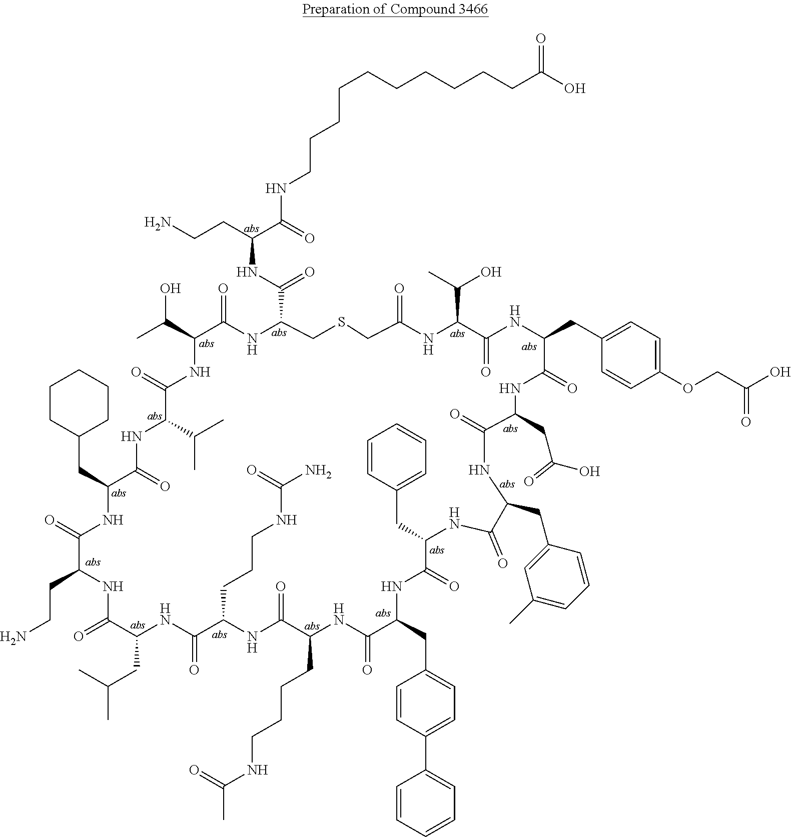 [SMILES: CC(=O)NCCCC[C@@H]1NC(=O)[C@H](Cc2ccc(-c3ccccc3)cc2)NC(=O)[C@H](Cc2ccccc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCNC(N)=O)NC1=O]

Compound 3466 was prepared on a 50 μmol scale. The yield of the product was 22.5 mg, and its estimated purity by LCMS analysis was 95.2%. Analysis condition B: Retention time=1.94 min; ESI-MS(+) m/z [M+3H]³⁺: 769.8.

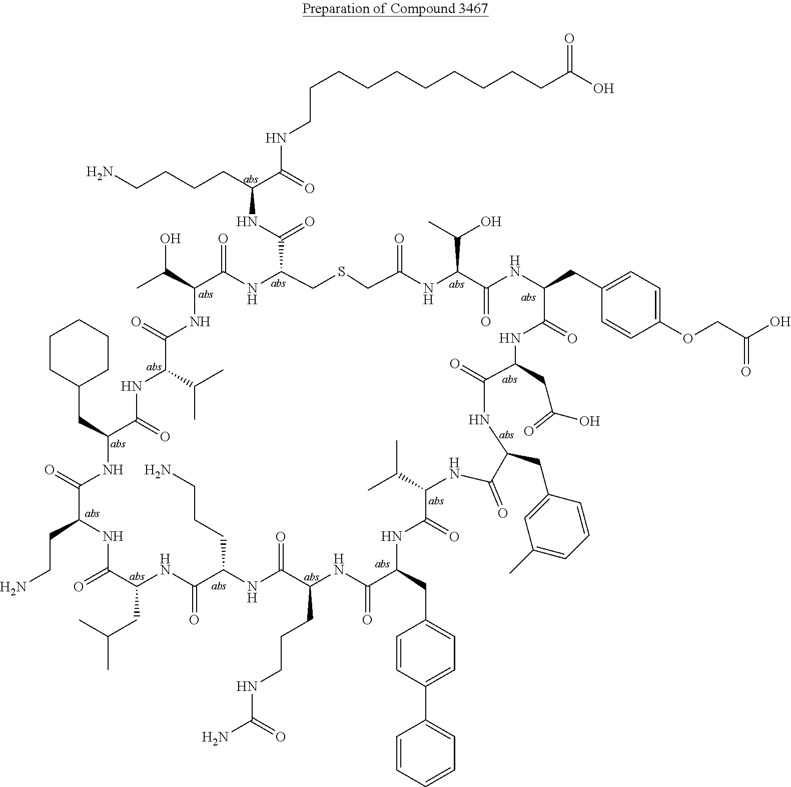 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3467 was prepared on a 50 μmol scale. The yield of the product was 56.7 mg, and its estimated purity by LCMS analysis was 95.3%. Analysis condition B: Retention time=1.75 min; ESI-MS(+) m/z [M+3H]³⁺: 745.

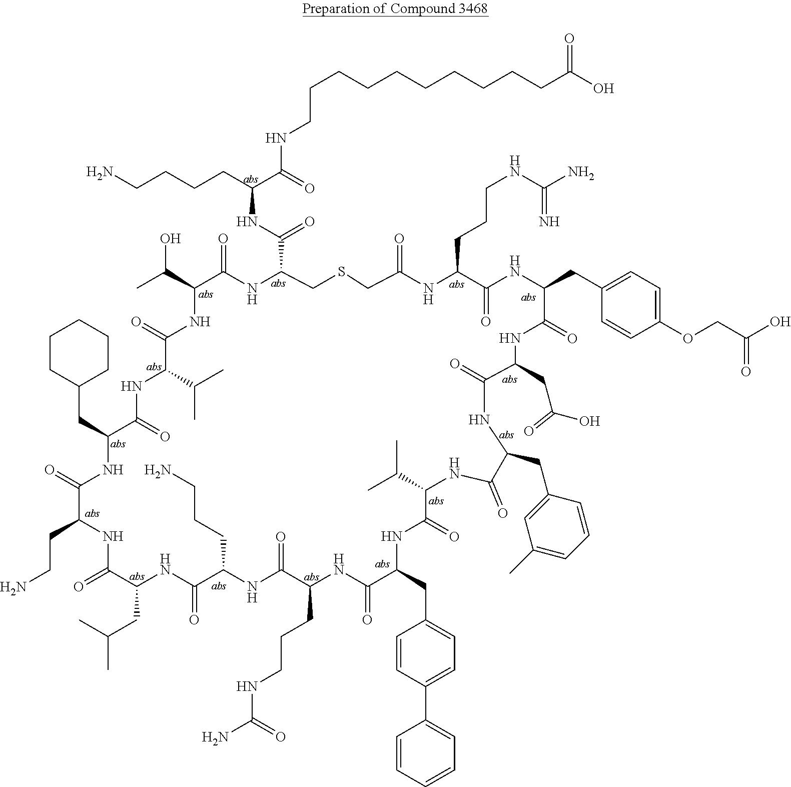 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3468 was prepared on a 50 μmol scale. The yield of the product was 43.6 mg, and its estimated purity by LCMS analysis was 95.5%. Analysis condition B: Retention time=1.6 min; ESI-MS(+) m/z [M+2H]²⁺: 1144.1.

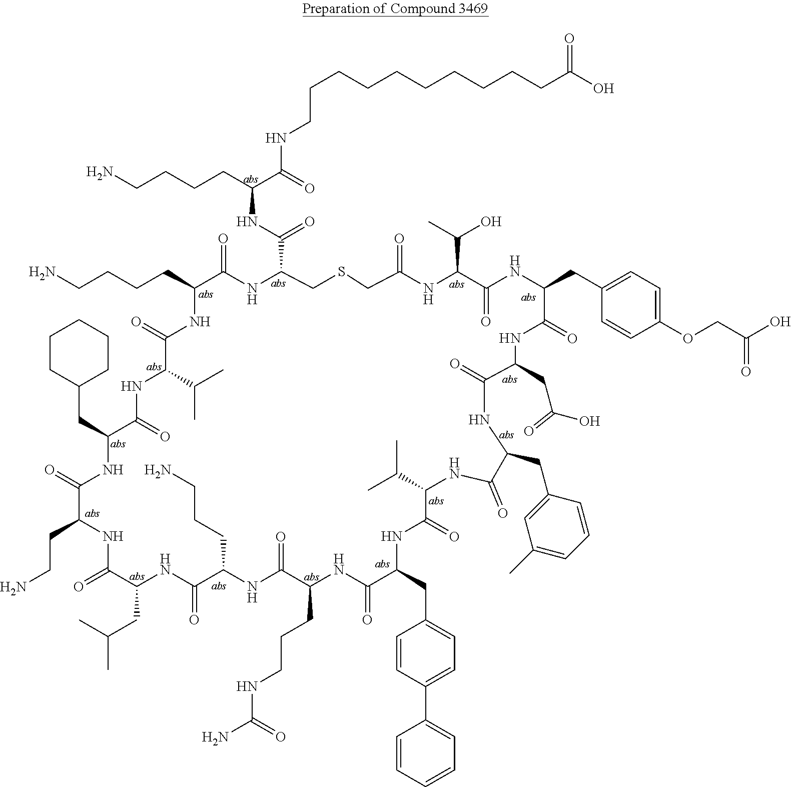 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCCCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3469 was prepared on a 50 μmol scale. The yield of the product was 25.2 mg, and its estimated purity by LCMS analysis was 96%. Analysis condition A: Retention time-1.57 min; ESI-MS(+) m/z [M+2H]²⁺: 1129.9.

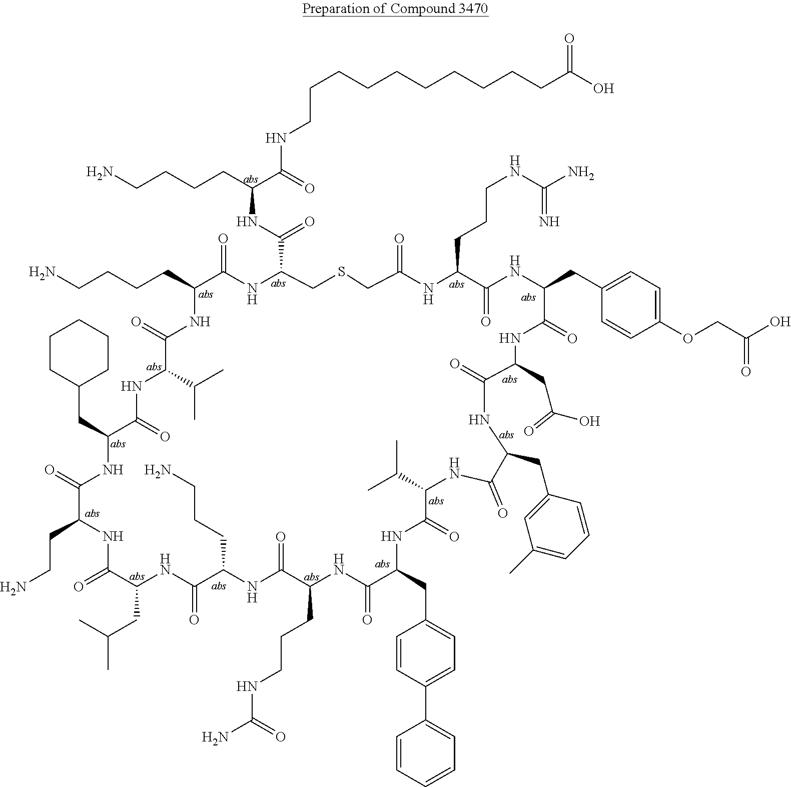 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3470 was prepared on a 50 μmol scale. The yield of the product was 13.5 mg, and its estimated purity by LCMS analysis was 95%. Analysis condition A: Retention time-1.51 min; ESI-MS(+) m/z [M+3H]³⁺: 771.9.

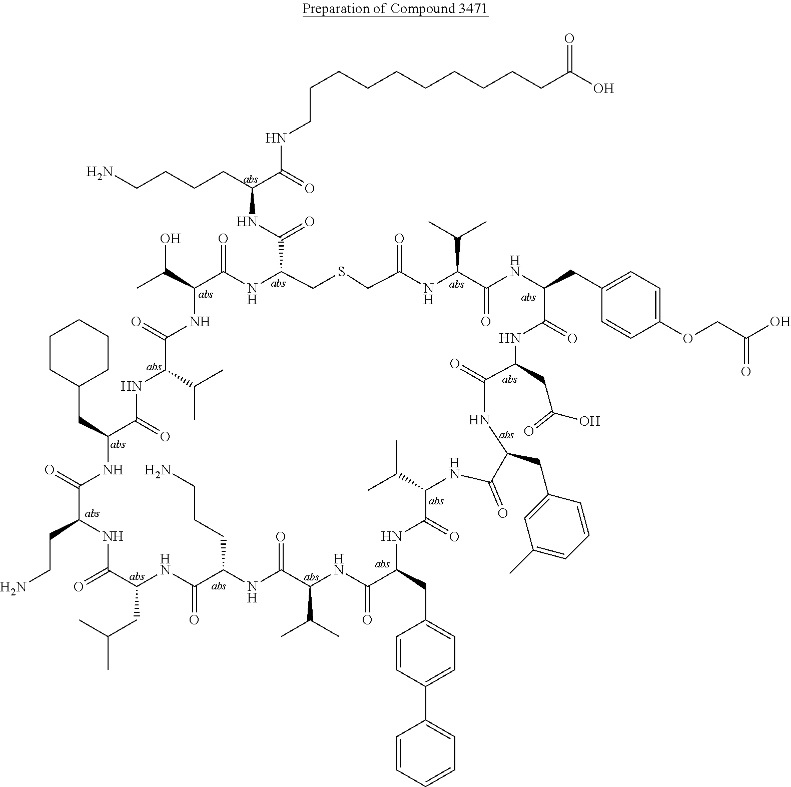 [SMILES: Cc1cccc(C[C@@H]2NC(=O)[C@H](CC(=O)O)NC(=O)[C@H](Cc3ccc(OCC(=O)O)cc3)NC(=O)[C@H](C(C)C)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3471 was prepared on a 50 μmol scale. The yield of the product was 7.7 mg, and its estimated purity by LCMS analysis was 96.5%. Analysis condition B: Retention time=1.62 min; ESI-MS(+) m/z [M+3H]³⁺: 725.5.

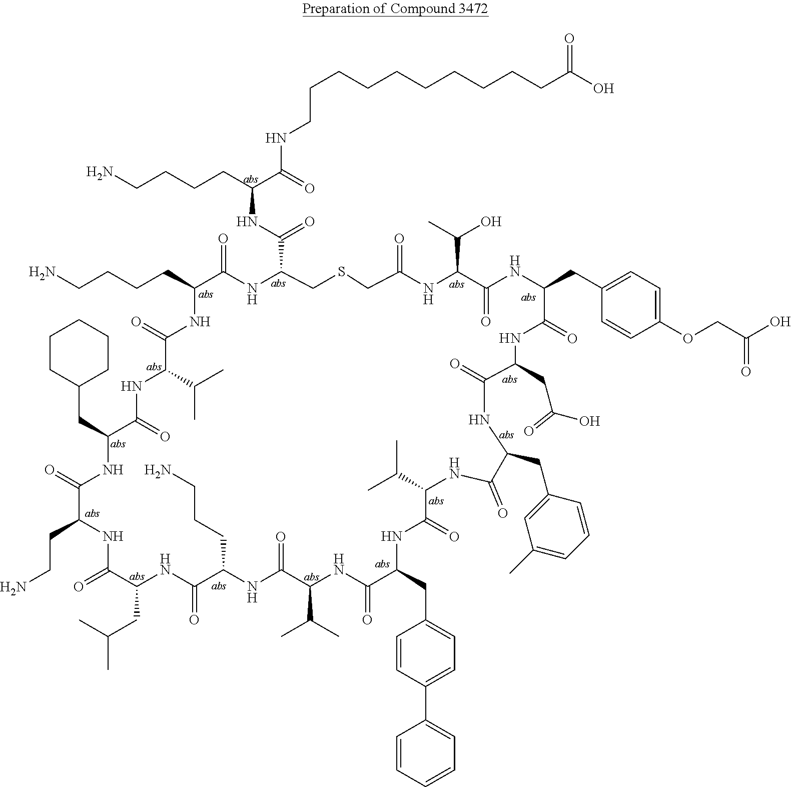 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCCCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3472 was prepared on a 50 μmol scale. The yield of the product was 6.7 mg, and its estimated purity by LCMS analysis was 98.7%. Analysis condition A: Retention time=1.64 min; ESI-MS(+) m/z [M+2H]²⁺: 1101.2.

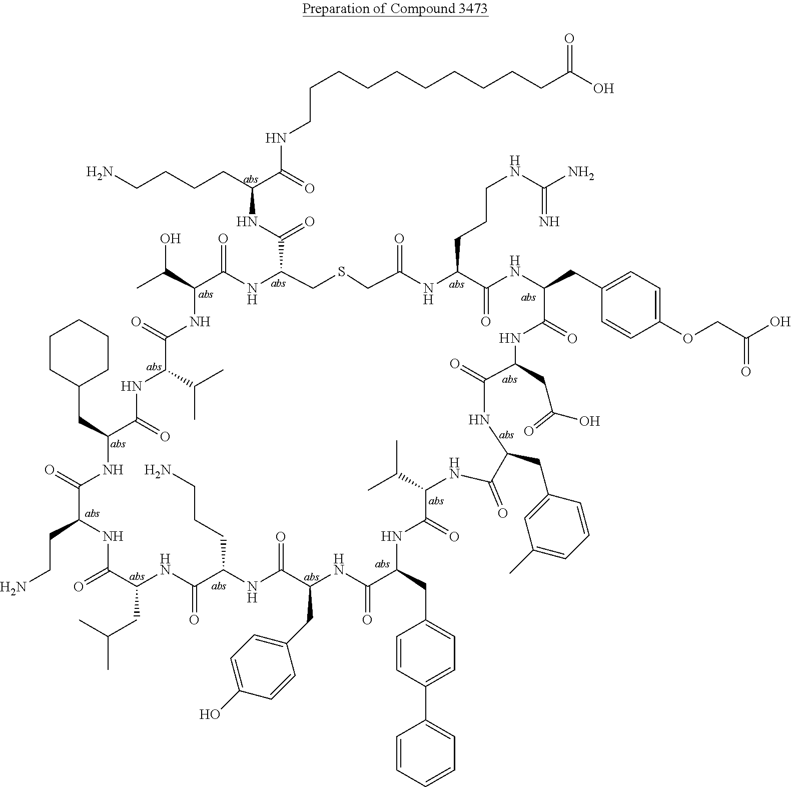 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3473 was prepared on a 50 μmol scale. The yield of the product was 8 mg, and its estimated purity by LCMS analysis was 97.8%. Analysis condition B: Retention time=1.47 min; ESI-MS(+) m/z [M+3H]³⁺: 765.1.

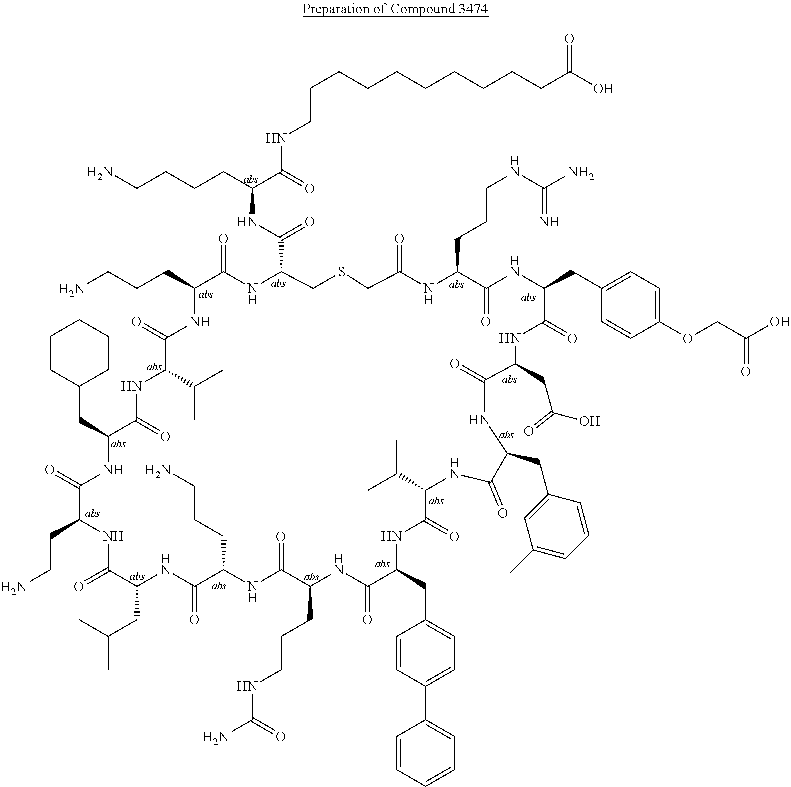 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3474 was prepared on a 50 μmol scale. The yield of the product was 23.5 mg, and its estimated purity by LCMS analysis was 97.1%. Analysis condition A: Retention time=1.54 min; ESI-MS(+) m/z [M+2H]²⁺: 1149.4.

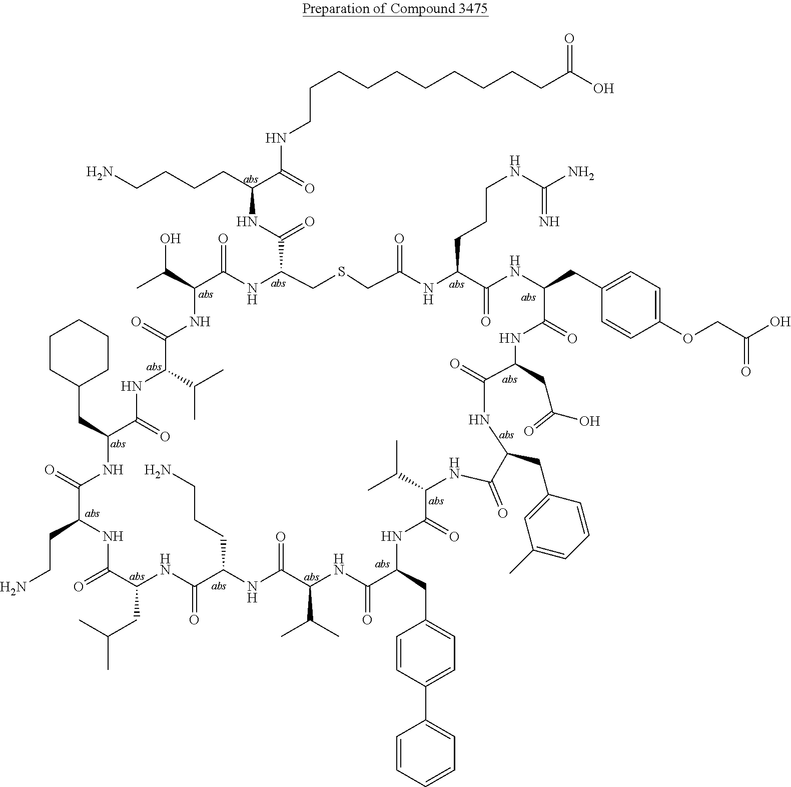 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3475 was prepared on a 50 μmol scale. The yield of the product was 18.4 mg, and its estimated purity by LCMS analysis was 96.4%. Analysis condition B: Retention time=1.56 min; ESI-MS(+) m/z [M+2H]²⁺: 1114.9.

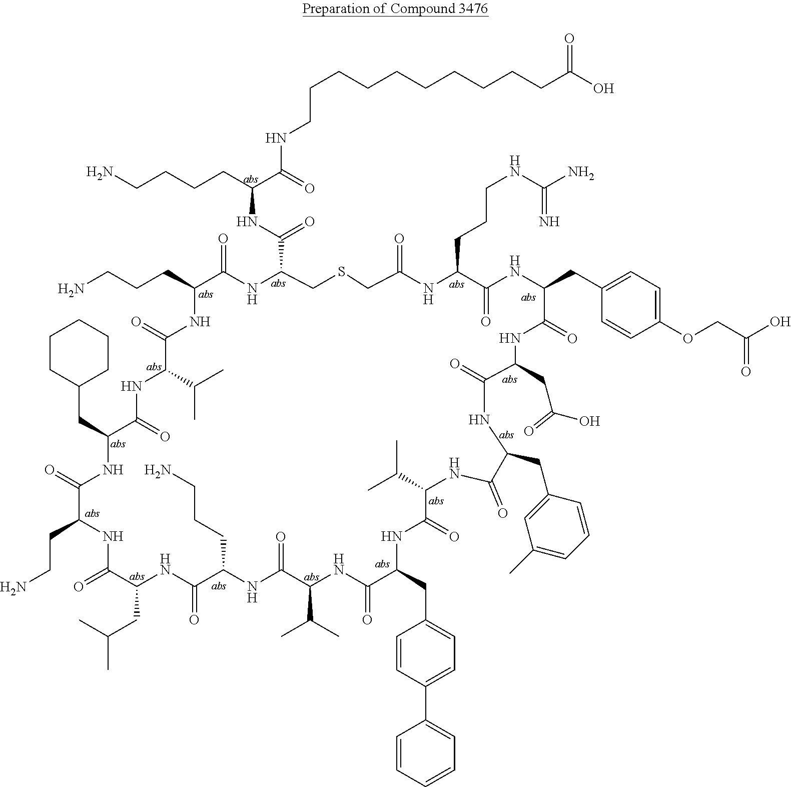 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3476 was prepared on a 50 μmol scale. The yield of the product was 11 mg, and its estimated purity by LCMS analysis was 100%. Analysis condition A: Retention time=1.6 min; ESI-MS(+) m/z [M+3H]³⁺: 753.

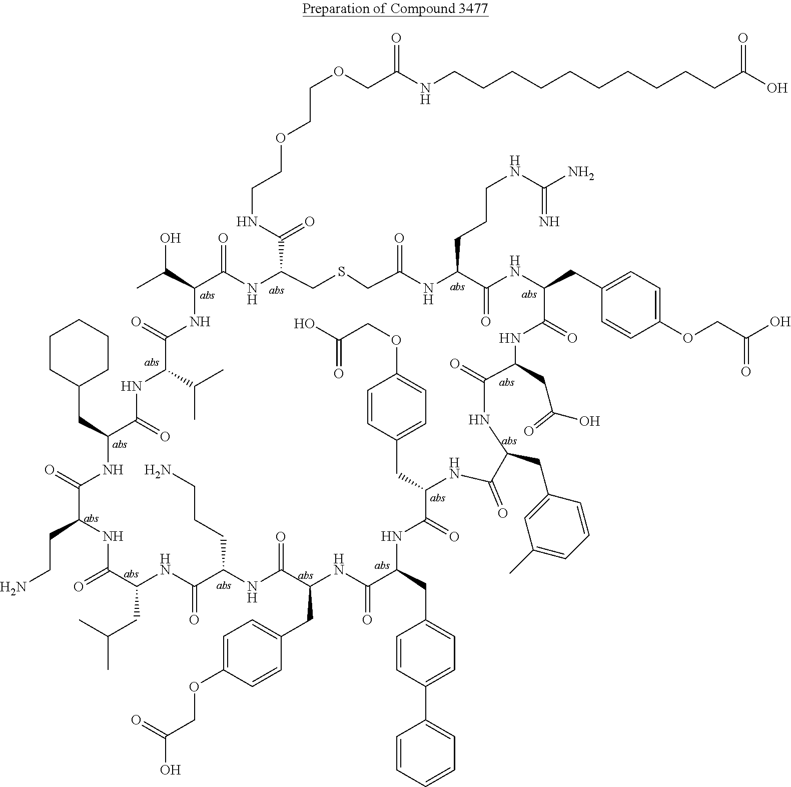 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3477 was prepared on a 50 μmol scale. The yield of the product was 25.6 mg, and its estimated purity by LCMS analysis was 100%. Analysis condition A: Retention time=1.56 min; ESI-MS(+) m/z [M+2H]²⁺: 1245.3.

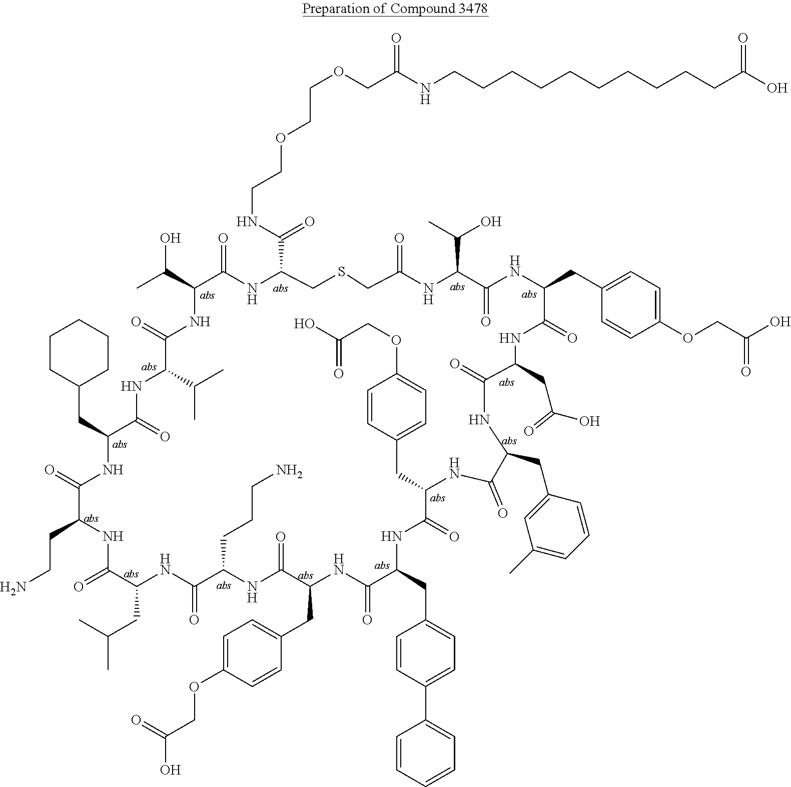 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3478 was prepared on a 50 μmol scale. The yield of the product was 43.1 mg, and its estimated purity by LCMS analysis was 95.4%. Analysis condition B: Retention time=1.92 min; ESI-MS(+) m/z [M+2H]²⁺: 1218.1.

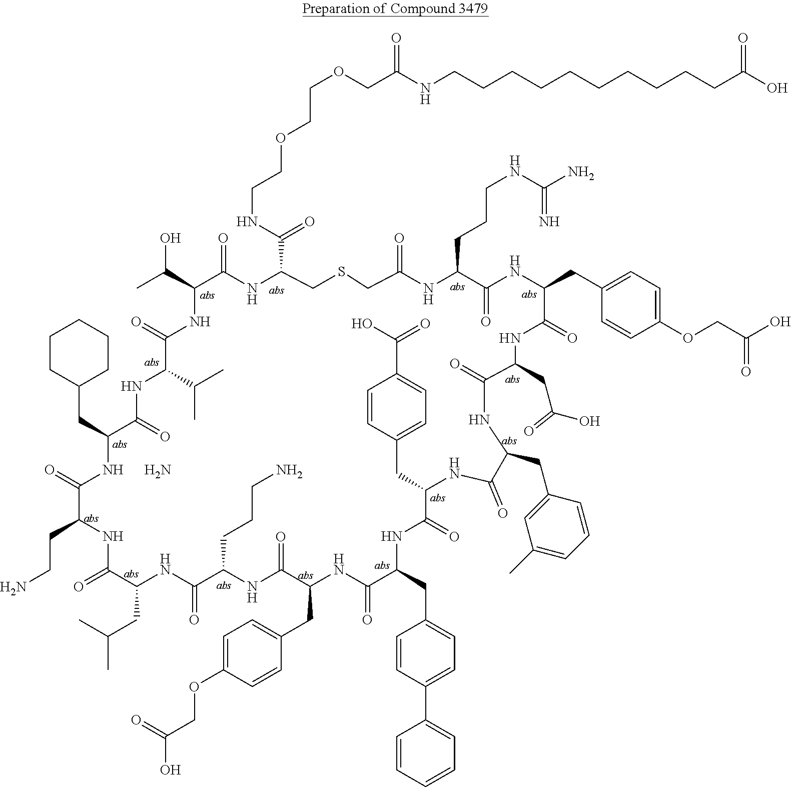

Compound 3479 was prepared on a 50 μmol scale. The yield of the product was 11.8 mg, and its estimated purity by LCMS analysis was 98.3%. Analysis condition B: Retention time=1.7 min; ESI-MS(+) m/z [M+3H]³⁺: 820.9.

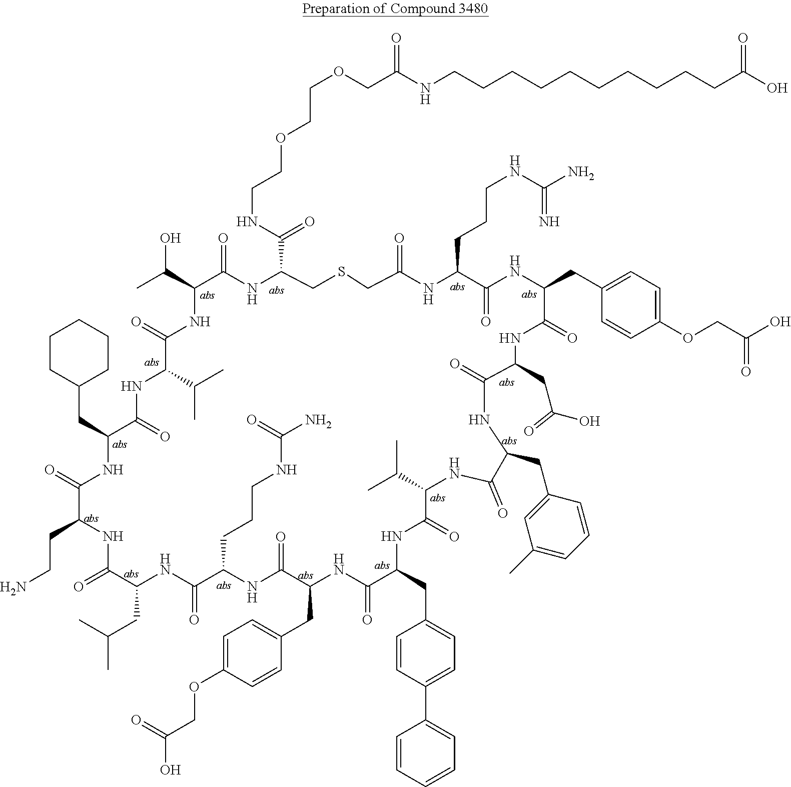 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 3480 was prepared on a 50 μmol scale. The yield of the product was 37.8 mg, and its estimated purity by LCMS analysis was 95.4%. Analysis condition B: Retention time=1.92 min; ESI-MS(+) m/z [M+2H]²⁺: 1206.2.

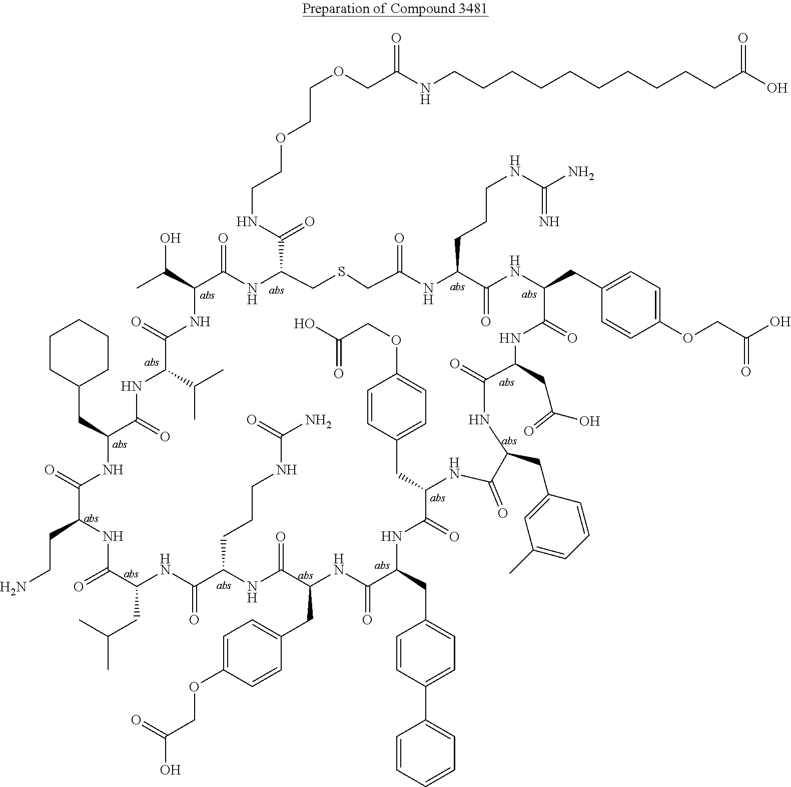 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3481 was prepared on a 50 μmol scale. The yield of the product was 33.2 mg, and its estimated purity by LCMS analysis was 97.7%. Analysis condition B: Retention time=1.91 min; ESI-MS(+) m/z [M+2H]²⁺: 1267.2.

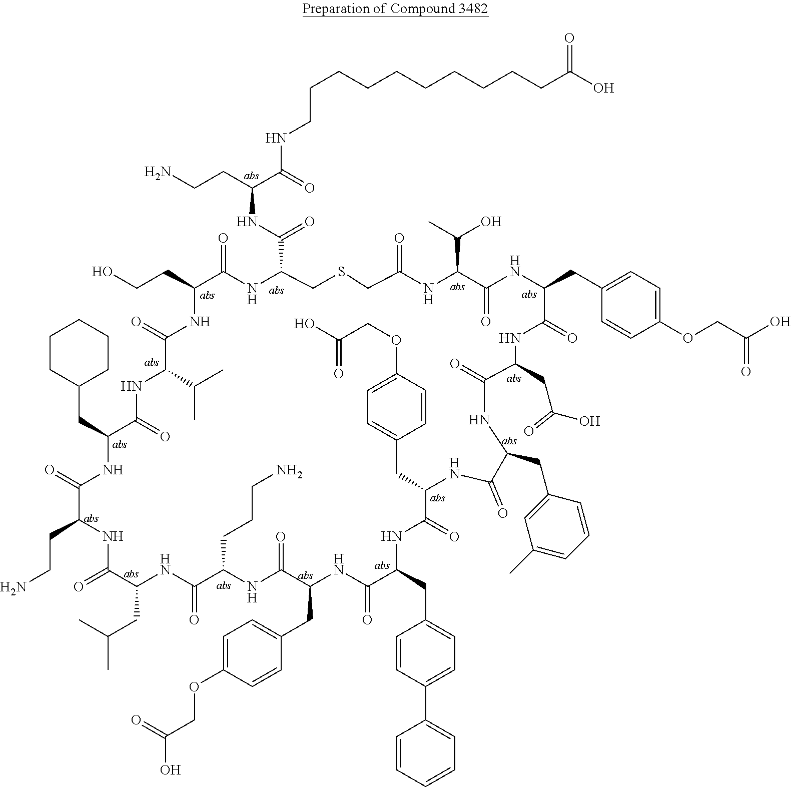 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3482 was prepared on a 50 μmol scale. The yield of the product was 8.5 mg, and its estimated purity by LCMS analysis was 91.3%. Analysis condition B: Retention time=1.64 min; ESI-MS(+) m/z [M+2H]²⁺: 1195.3.

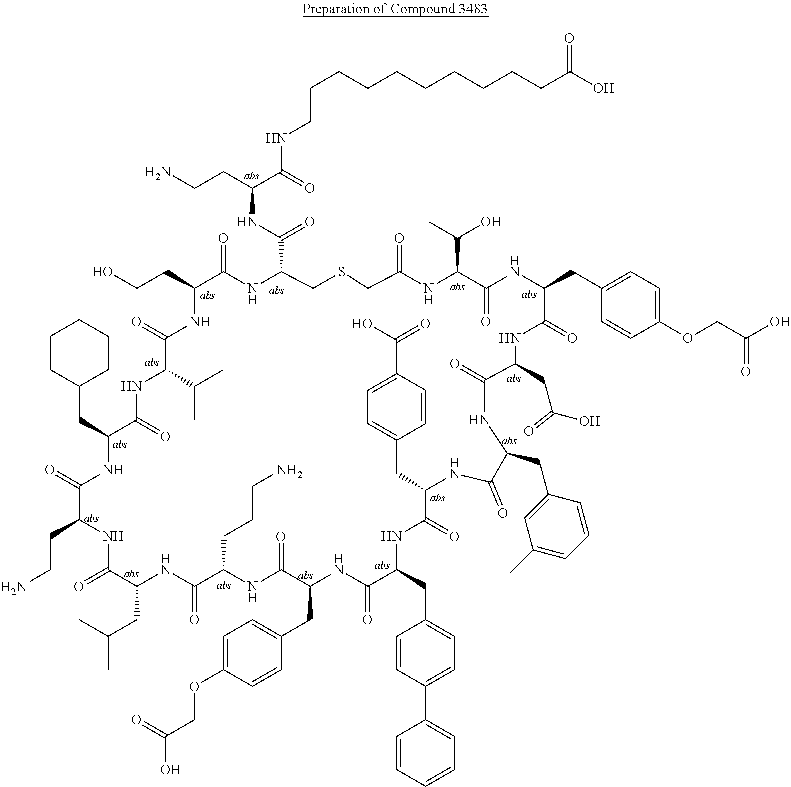 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CCO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3483 was prepared on a 50 μmol scale. The yield of the product was 6.6 mg, and its estimated purity by LCMS analysis was 91.9%. Analysis condition B: Retention time-1.65 min; ESI-MS(+) m/z [M+3H]³⁺: 787.9.

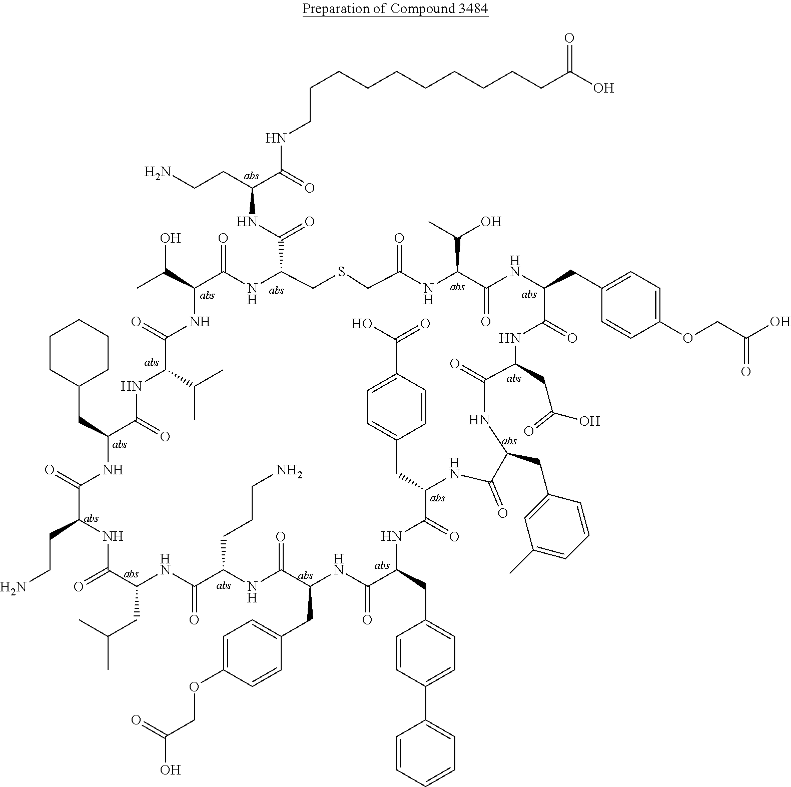 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3484 was prepared on a 50 μmol scale. The yield of the product was 4.4 mg, and its estimated purity by LCMS analysis was 96%. Analysis condition A: Retention time=1.47 min; ESI-MS(+) m/z [M+3H]³⁺: 787.4.

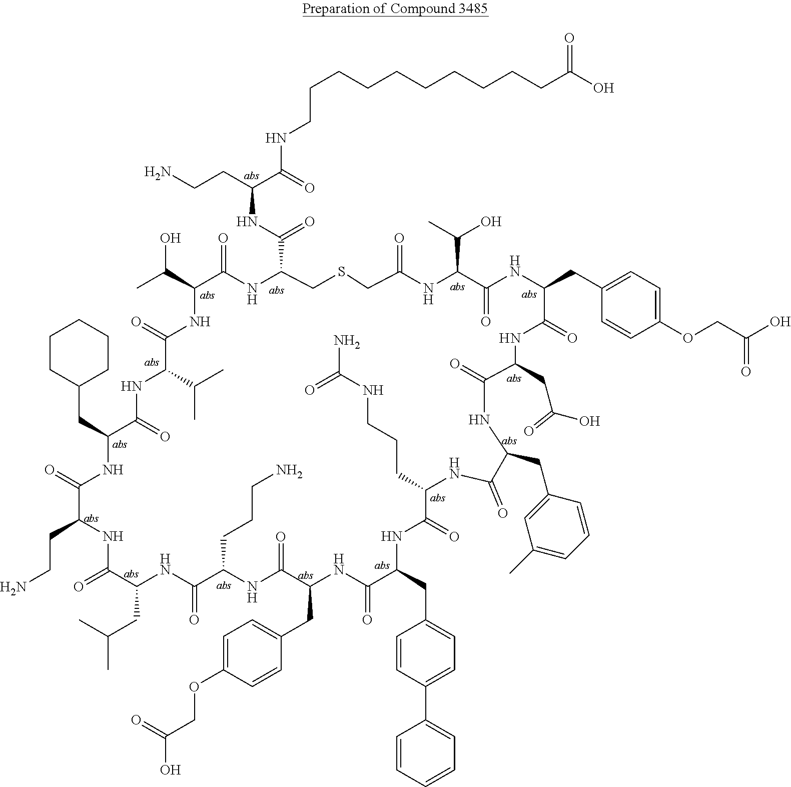 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](CCCNC(N)=O)NC2=O)c1]

Compound 3485 was prepared on a 50 μmol scale. The yield of the product was 5.9 mg, and its estimated purity by LCMS analysis was 95.3%. Analysis condition B: Retention time=1.65 min; ESI-MS(+) m/z [M+2H]²⁺: 1163.7.

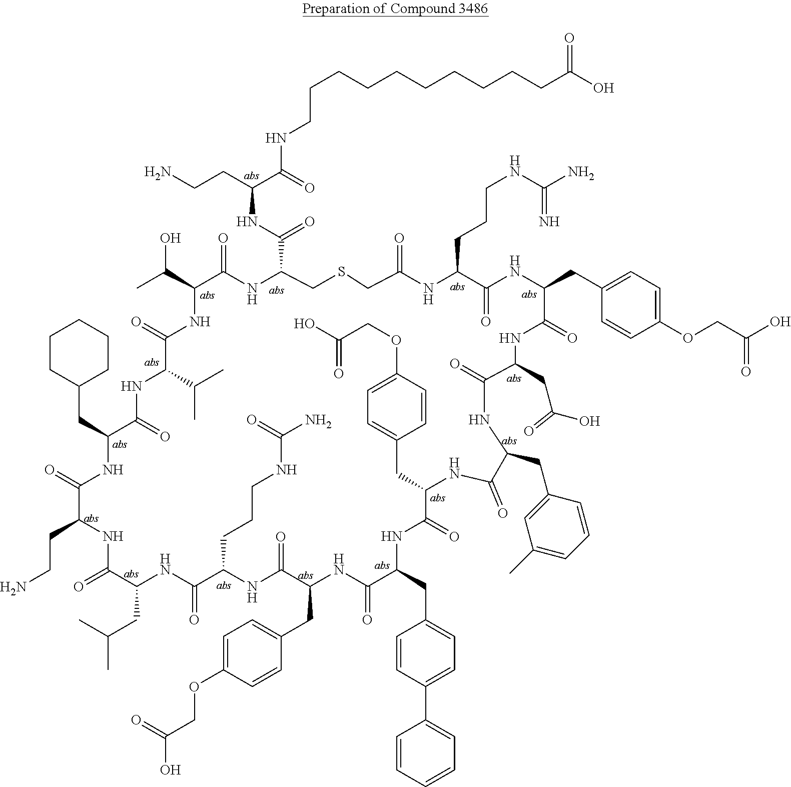 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3486 was prepared on a 50 μmol scale. The yield of the product was 9.7 mg, and its estimated purity by LCMS analysis was 95.1%. Analysis condition A: Retention time=1.39 min; ESI-MS(+) m/z [M+2H]²⁺: 1245.

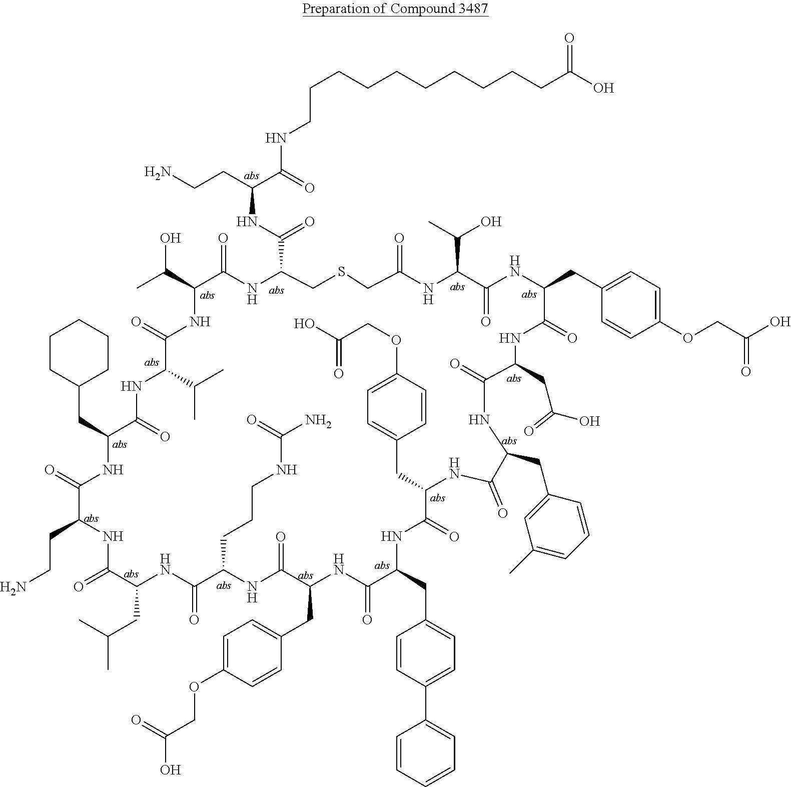 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3487 was prepared on a 50 μmol scale. The yield of the product was 6.2 mg, and its estimated purity by LCMS analysis was 95.3%. Analysis condition A: Retention time=1.4 min; ESI-MS(+) m/z [M+2H]²⁺: 1217.5.

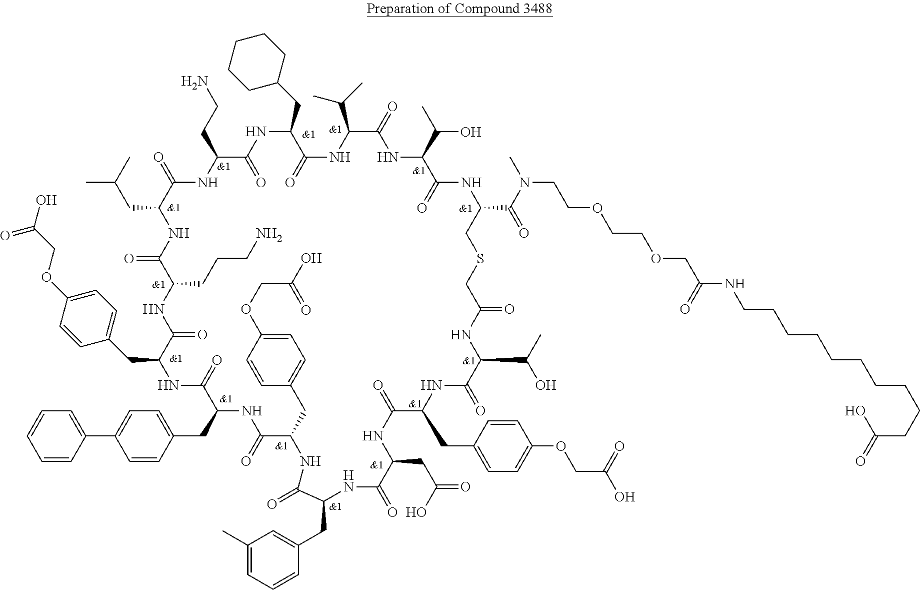 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N(C)CCOCCOCC(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3488 was prepared on a 50 μmol scale. The yield of the product was 2.3 mg, and its estimated purity by LCMS analysis was 95.6%. Analysis condition B: Retention time=1.87 min; ESI-MS(+) m/z [M+2H]²⁺: 1210.

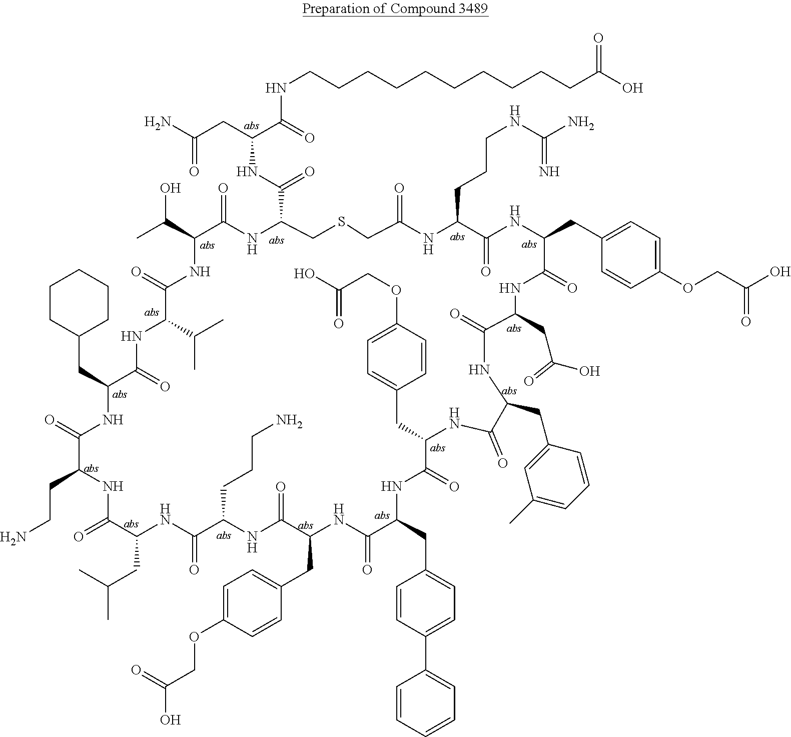 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3489 was prepared on a 50 μmol scale. The yield of the product was 13.3 mg, and its estimated purity by LCMS analysis was 95.8%. Analysis condition B: Retention time=1.69 min; ESI-MS(+) m/z [M+2H]²⁺: 1230.2.

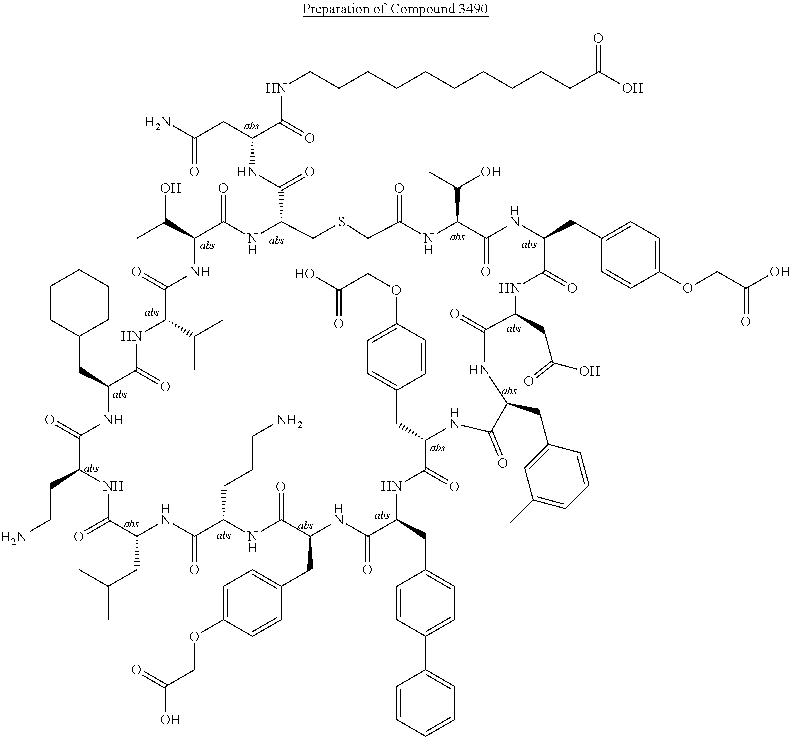 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3490 was prepared on a 50 μmol scale. The yield of the product was 25.4 mg, and its estimated purity by LCMS analysis was 97.2%. Analysis condition B: Retention time=1.75 min; ESI-MS(+) m/z [M+2H]²⁺: 1202.9.

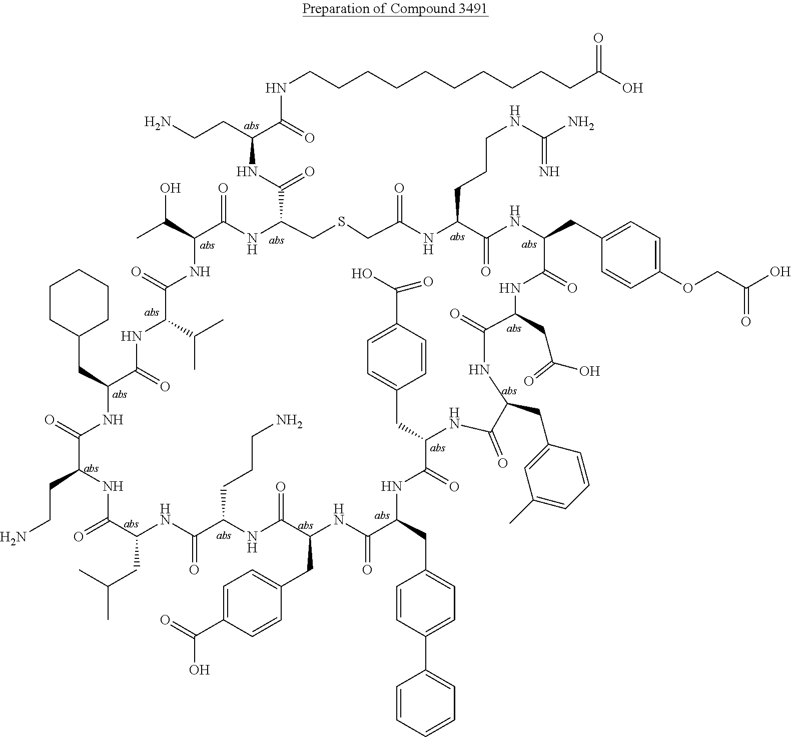 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3491 was prepared on a 50 μmol scale. The yield of the product was 8.9 mg, and its estimated purity by LCMS analysis was 86.5%. Analysis condition A: Retention time=1.57 min; ESI-MS(+) m/z [M+2H]²⁺: 1193.

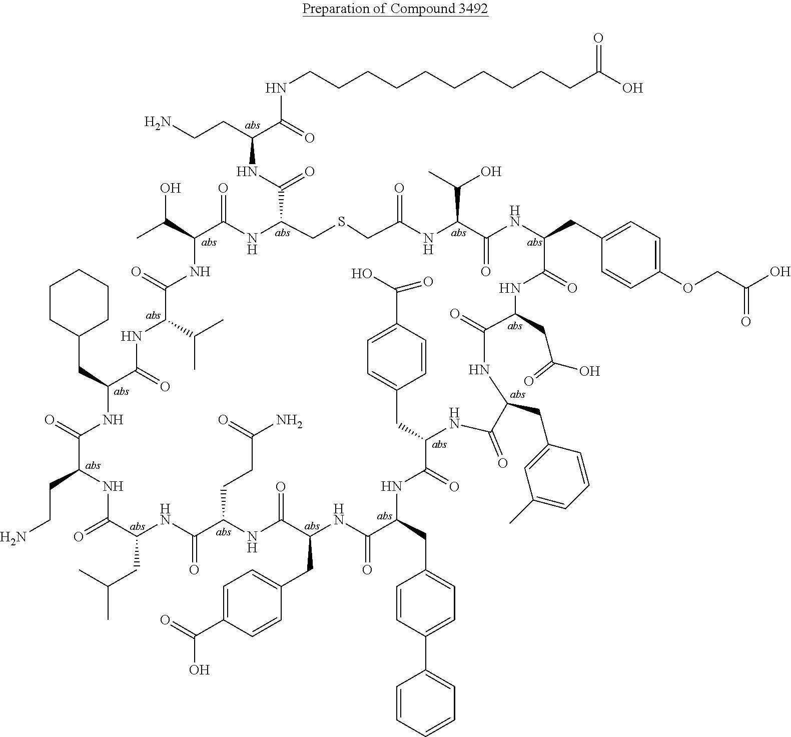 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3492 was prepared on a 50 μmol scale. The yield of the product was 4.8 mg, and its estimated purity by LCMS analysis was 96.5%. Analysis condition A: Retention time=1.43 min; ESI-MS(+) m/z [M+3H]³⁺: 777.8.

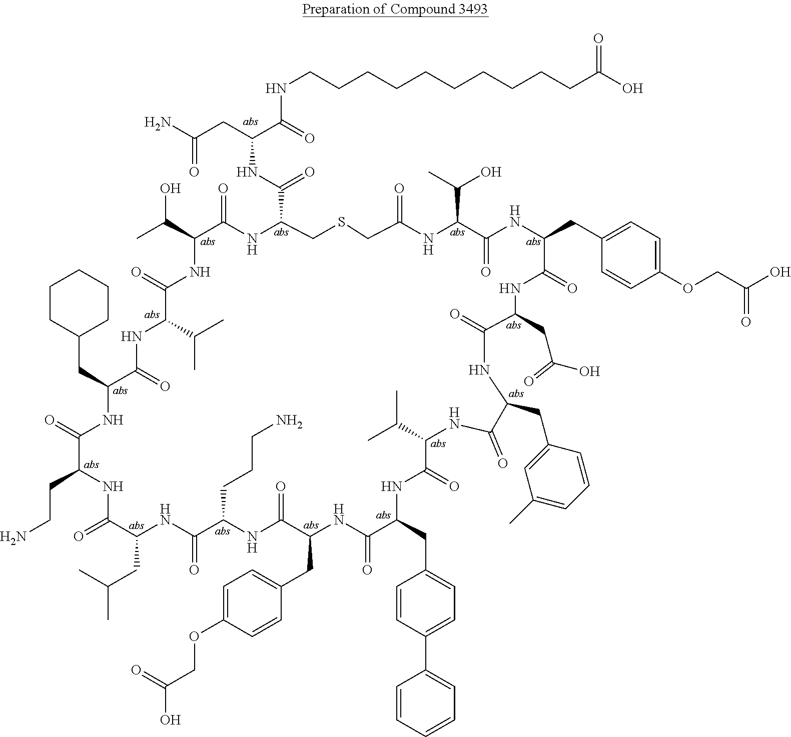 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 3493 was prepared on a 50 μmol scale. The yield of the product was 13.6 mg, and its estimated purity by LCMS analysis was 100%. Analysis condition A: Retention time=1.53 min; ESI-MS(+) m/z [M+2H]²⁺: 1141.3.

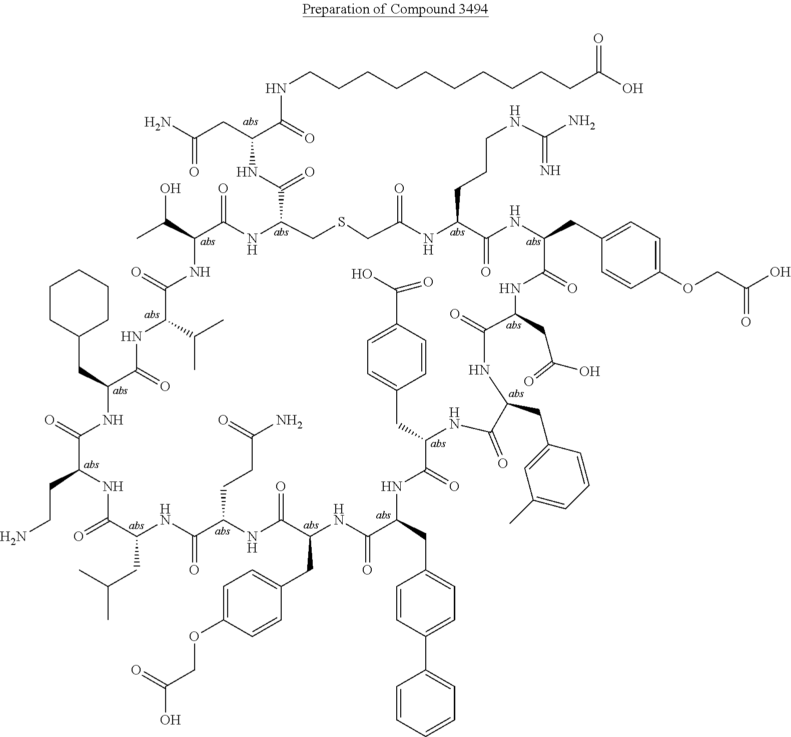 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3494 was prepared on a 50 μmol scale. The yield of the product was 10.3 mg, and its estimated purity by LCMS analysis was 100%. Analysis condition A: Retention time=1.54 min; ESI-MS(+) m/z [M+2H]²⁺: 1169.3.

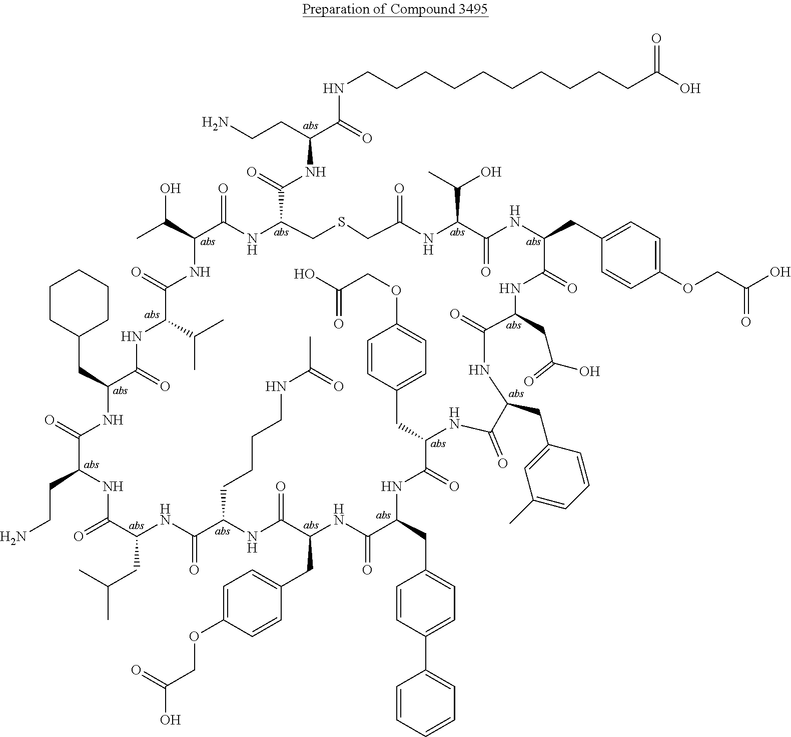 [SMILES: CC(=O)NCCCC[C@@H]1NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC1=O]

Compound 3495 was prepared on a 50 μmol scale. The yield of the product was 8 mg, and its estimated purity by LCMS analysis was 96.5%. Analysis condition B: Retention time=1.79 min; ESI-MS(+) m/z [M+2H]²⁺: 1223.3.

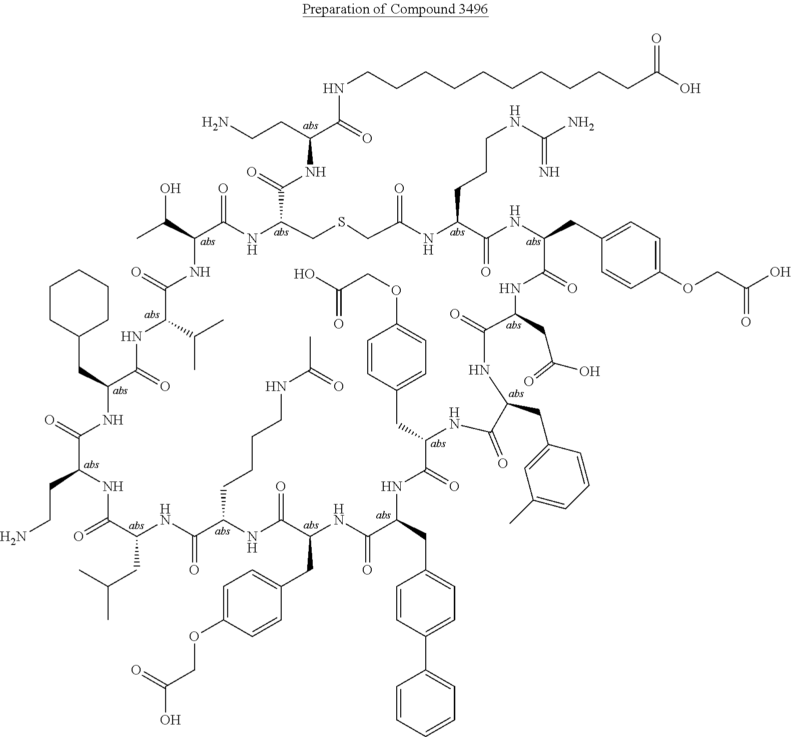 [SMILES: CC(=O)NCCCC[C@@H]1NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC1=O]

Compound 3496 was prepared on a 50 μmol scale. The yield of the product was 5.7 mg, and its estimated purity by LCMS analysis was 100%. Analysis condition A: Retention time=1.54 min; ESI-MS(+) m/z [M+2H]²⁺: 1251.1.

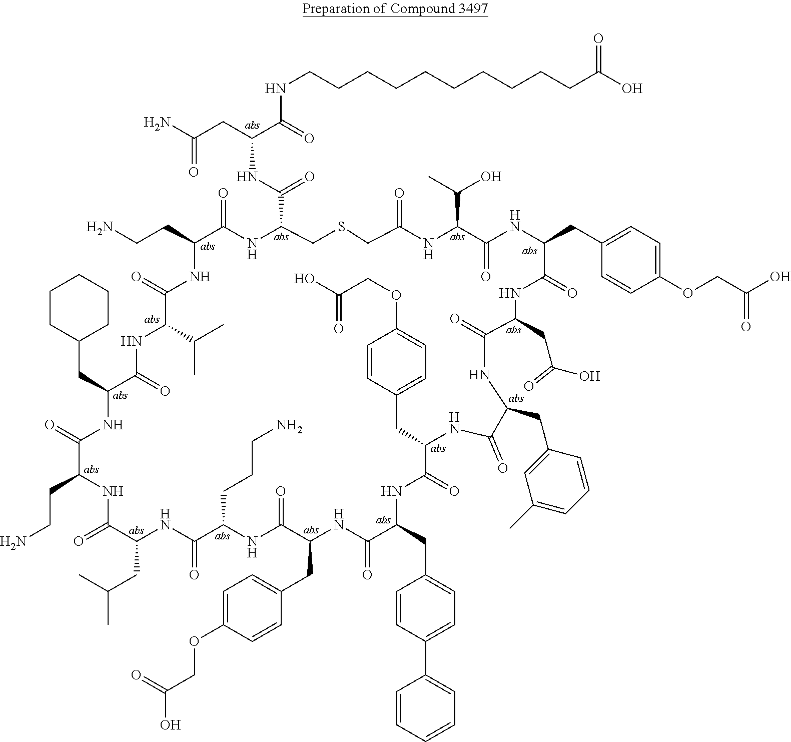

Compound 3497 was prepared on a 50 μmol scale. The yield of the product was 11.3 mg, and its estimated purity by LCMS analysis was 100%. Analysis condition A: Retention time=1.4 min; ESI-MS(+) m/z [M+2H]²⁺: 1202.1.

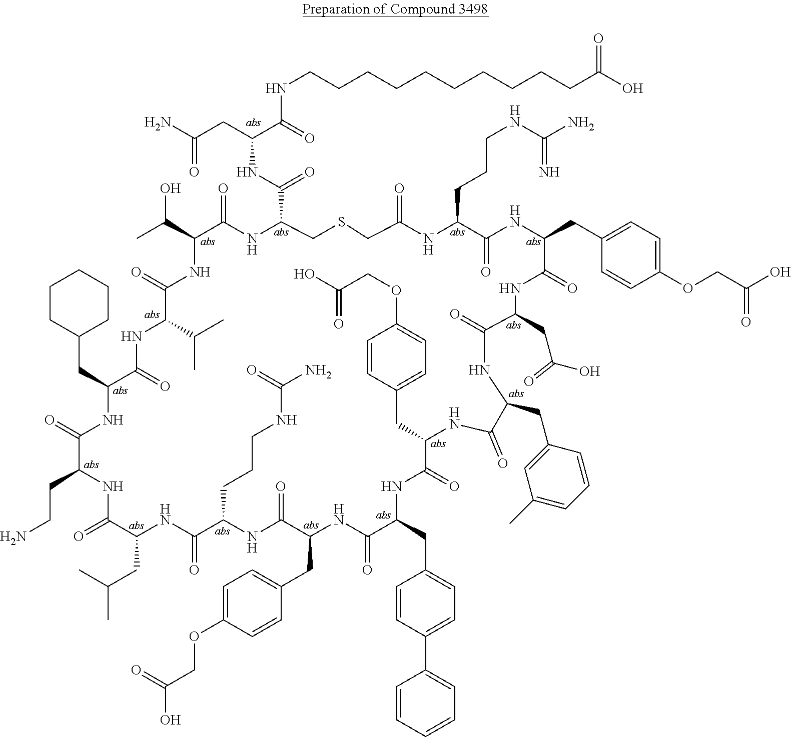 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3498 was prepared on a 50 μmol scale. The yield of the product was 3.9 mg, and its estimated purity by LCMS analysis was 92.2%. Analysis condition B: Retention time=1.78 min; ESI-MS(+) m/z [M+2H]²⁺: 1252.

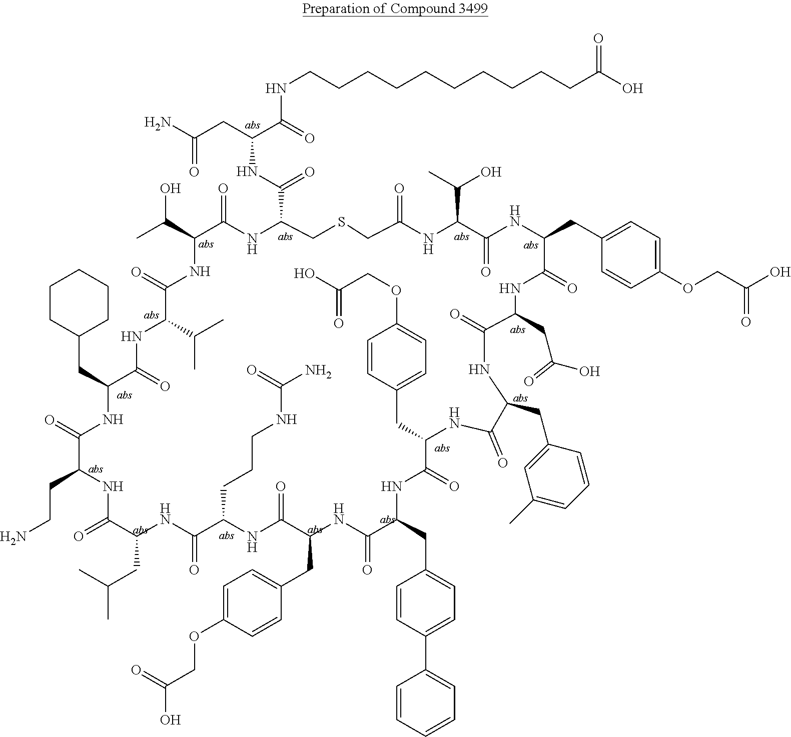 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3499 was prepared on a 50 μmol scale. The yield of the product was 12.6 mg, and its estimated purity by LCMS analysis was 100%. Analysis condition A: Retention time=1.38 min; ESI-MS(+) m/z [M+2H]²⁺: 1224.1.

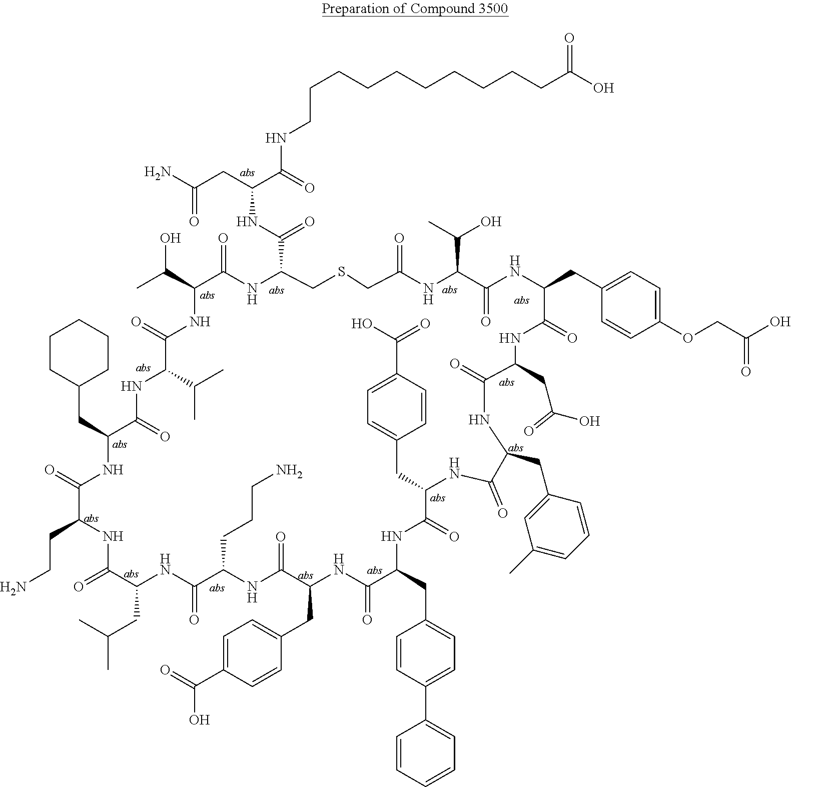 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3500 was prepared on a 50 μmol scale. The yield of the product was 10.2 mg, and its estimated purity by LCMS analysis was 96.1%. Analysis condition B: Retention time=1.73 min; ESI-MS(+) m/z [M+2H]²⁺: 1172.2.

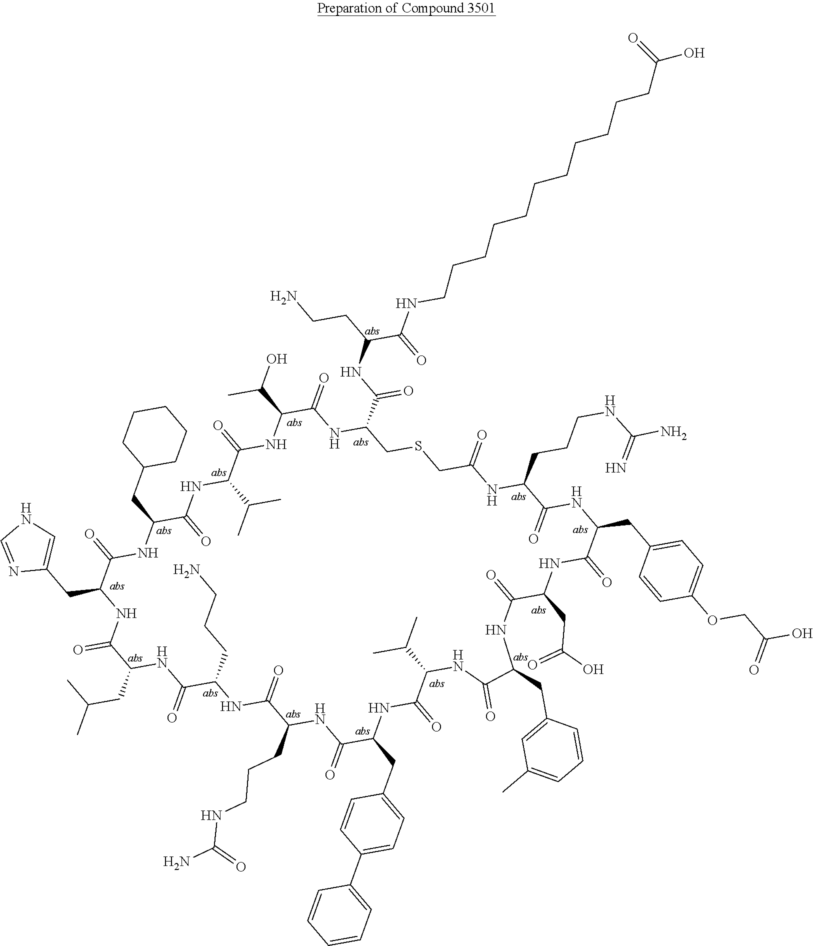

Compound 3501 was prepared on a 50 μmol scale. The yield of the product was 7.4 mg, and its estimated purity by LCMS analysis was 100%. Analysis condition A: Retention time=1.63 min; ESI-MS(+) m/z [M+2H]²⁺: 1155.3.

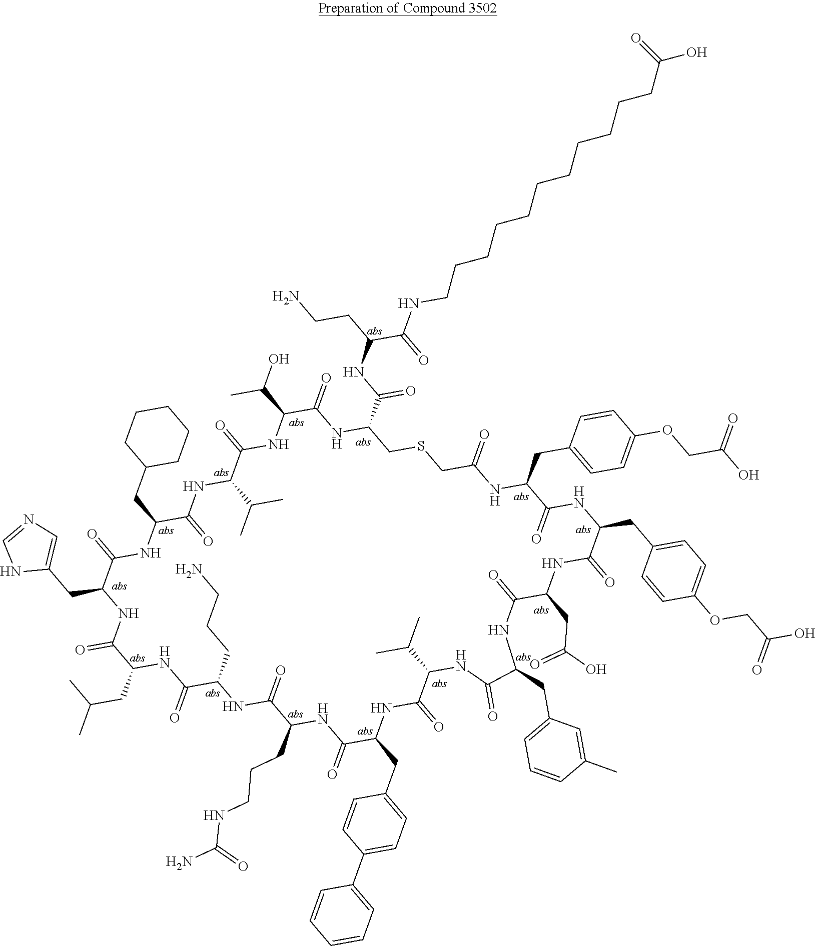

Compound 3502 was prepared on a 50 μmol scale. The yield of the product was 8.2 mg, and its estimated purity by LCMS analysis was 100%. Analysis condition A: Retention time=1.57 min; ESI-MS(+) m/z [M+2H]²⁺: 1188.

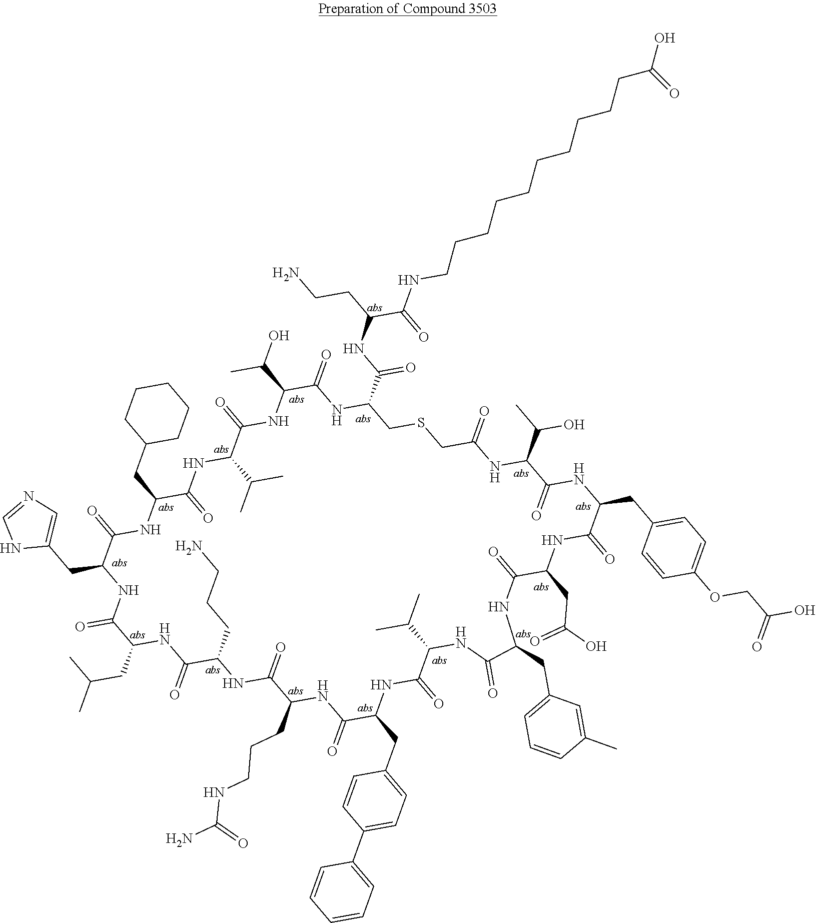

Compound 3503 was prepared on a 50 μmol scale. The yield of the product was 36.3 mg, and its estimated purity by LCMS analysis was 95.3%. Analysis condition A: Retention time=1.62 min; ESI-MS(+) m/z [M+2H]²⁺: 1121.2.

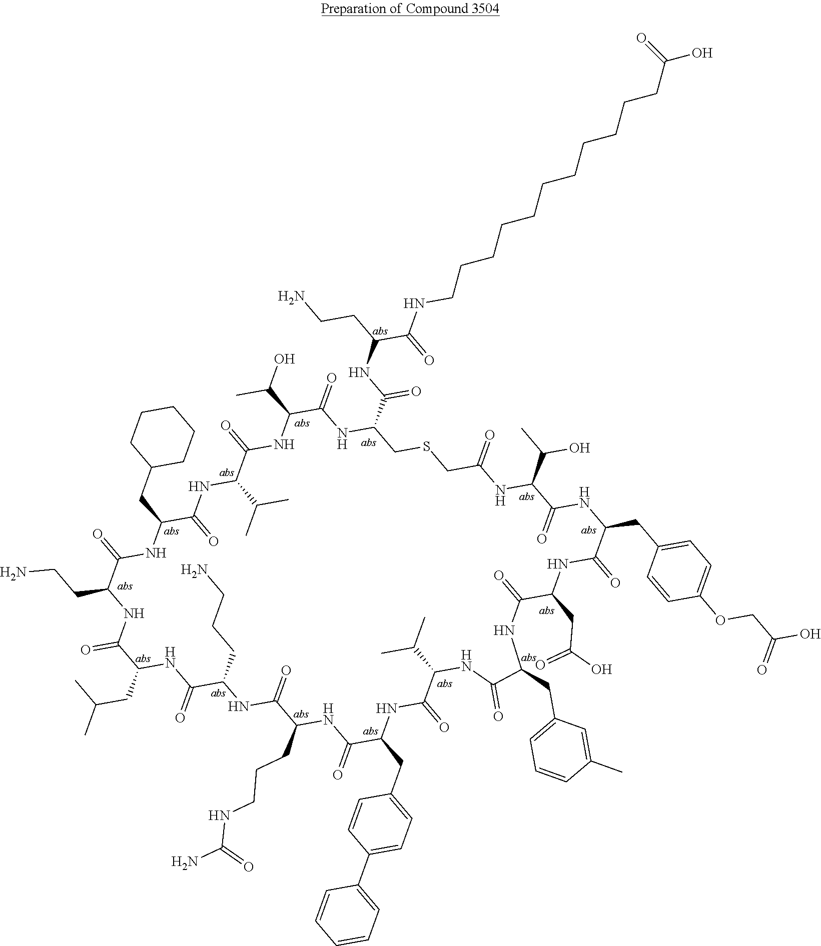

Compound 3504 was prepared on a 50 μmol scale. The yield of the product was 3.9 mg, and its estimated purity by LCMS analysis was 91.8%. Analysis condition A: Retention time=1.58 min; ESI-MS(+) m/z [M+3H]³⁺: 740.1.

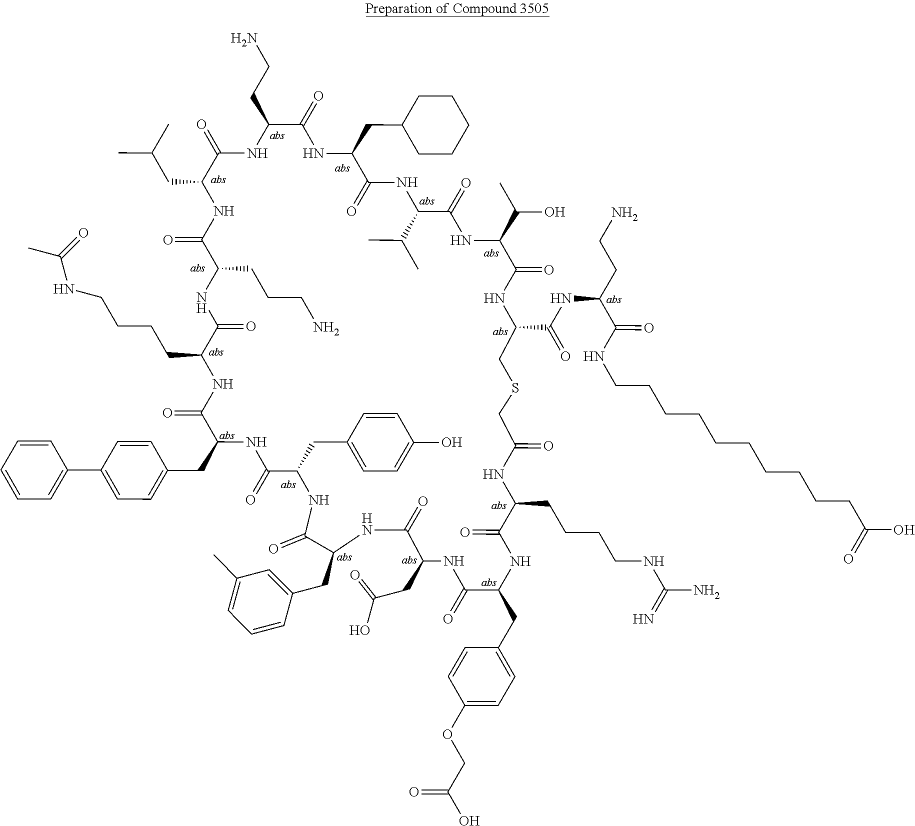 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3505 was prepared on a 50 μmol scale. The yield of the product was 4 mg, and its estimated purity by LCMS analysis was 100%. Analysis condition A: Retention time=1.84 min; ESI-MS(+) m/z [M+3H]³⁺: 784.

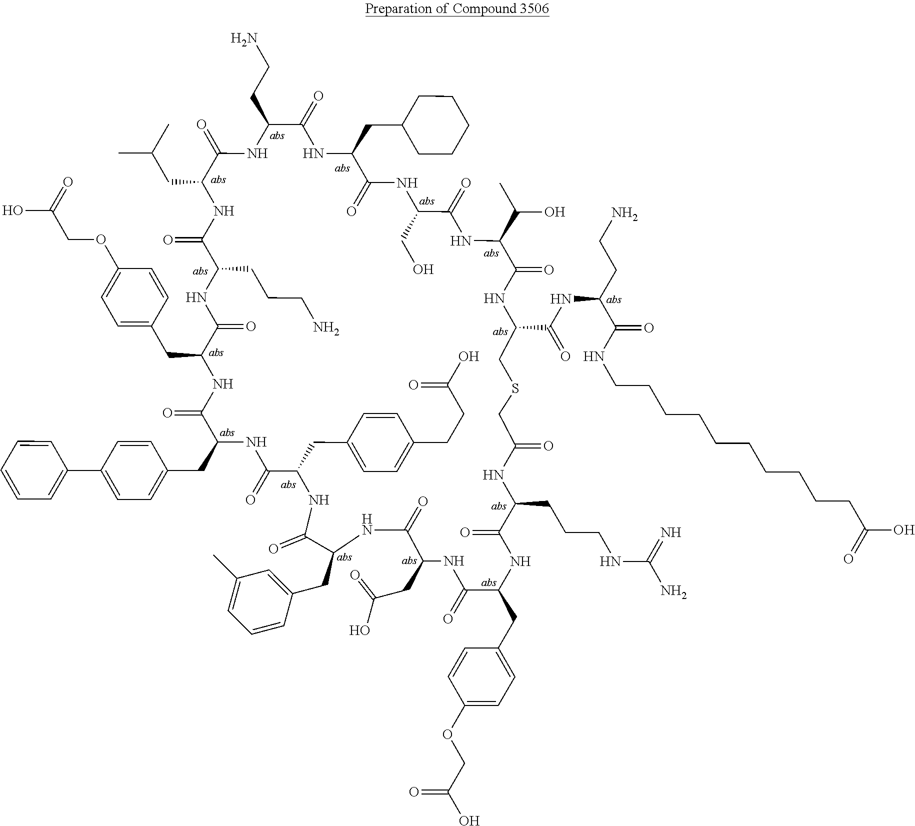 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(CCC(=O)O)cc3)NC2=O)c1]

Compound 3506 was prepared on a 50 μmol scale. The yield of the product was 32.4 mg, and its estimated purity by LCMS analysis was 95.5%. Analysis condition A: Retention time=1.51 min; ESI-MS(+) m/z [M+2H]²⁺: 1217.1.

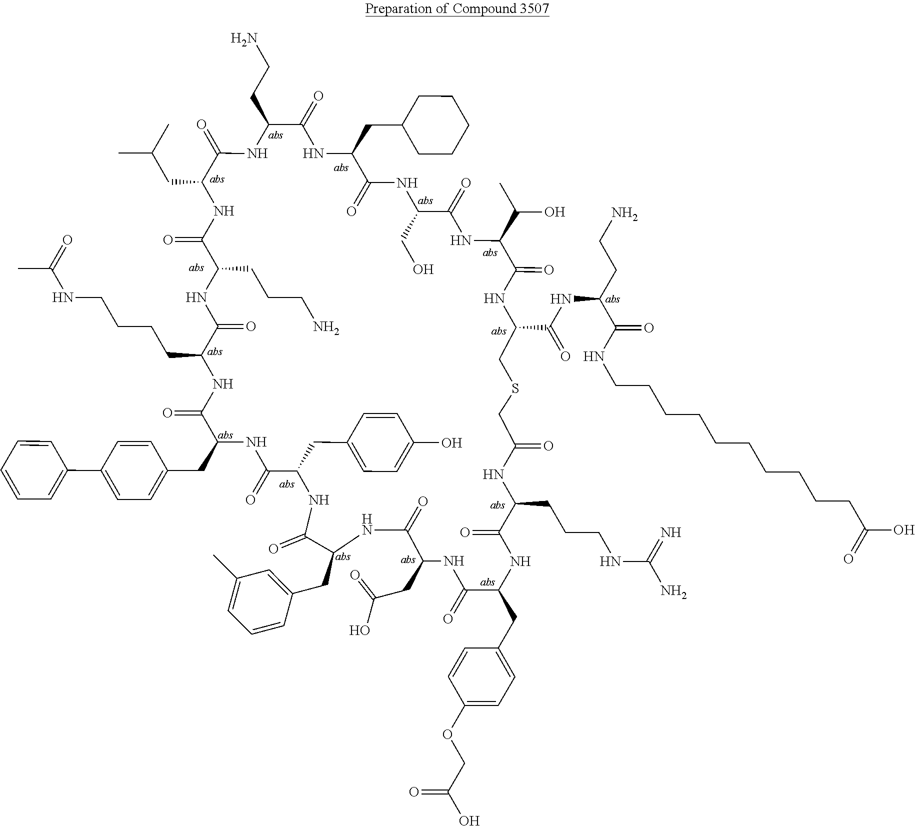 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](CO)NC(=O)[C@H](CC2CCCCC2)NC(=O)[C@H](CCN)NC(=O)[C@@H](CC(C)C)NC(=O)[C@H](CCCN)NC1=O]

Compound 3507 was prepared on a 50 μmol scale. The yield of the product was 15.4 mg, and its estimated purity by LCMS analysis was 100%. Analysis condition A: Retention time=2.05 min; ESI-MS(+) m/z [M+3H]³⁺: 775.1.

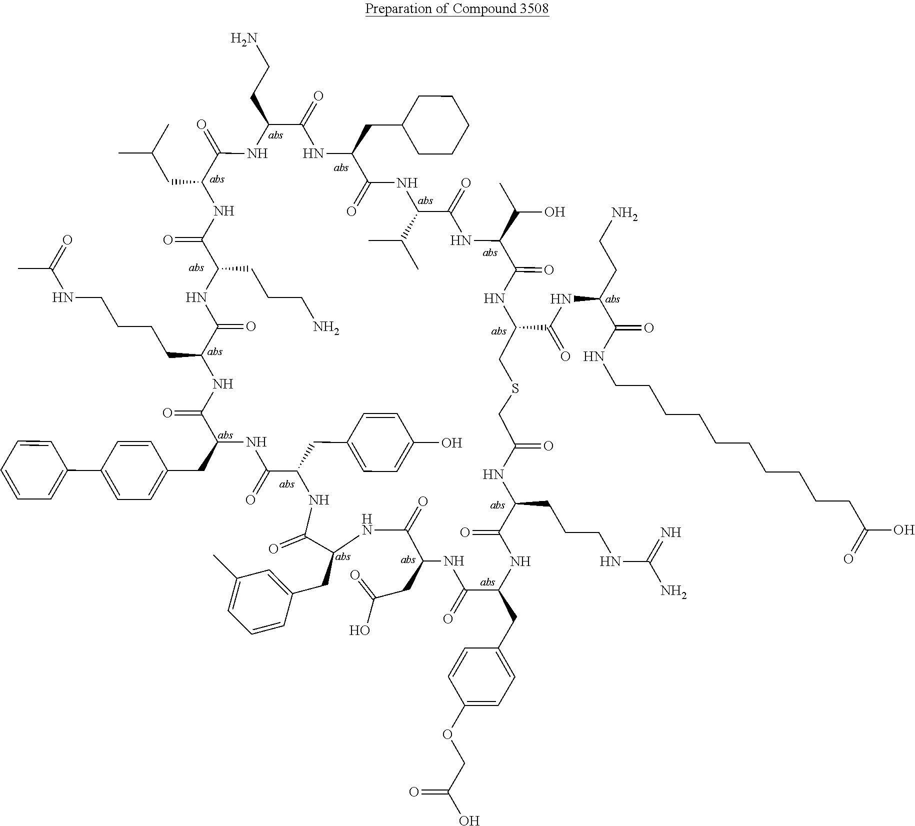 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3508 was prepared on a 50 μmol scale. The yield of the product was 9.1 mg, and its estimated purity by LCMS analysis was 99.3%. Analysis condition B: Retention time=1.46 min; ESI-MS(+) m/z [M+3H]³⁺: 779.1.

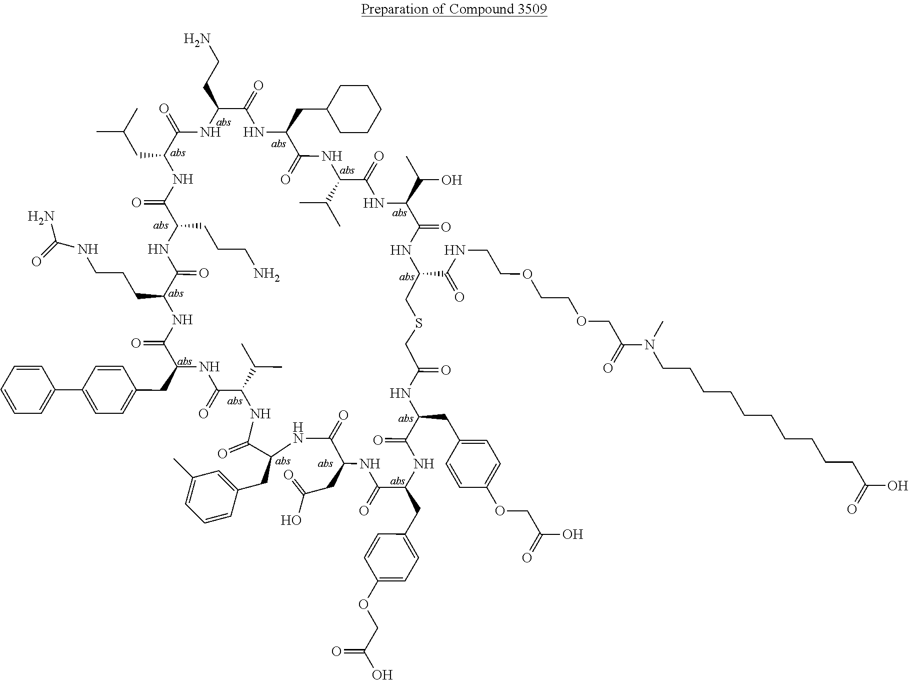 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCCOCCOCC(=O)N(C)CCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3509 was prepared on a 50 μmol scale. The yield of the product was 0.7 mg, and its estimated purity by LCMS analysis was 95.2%. Analysis condition A: Retention time=1.67 min; ESI-MS(+) m/z [M+2H]²⁺: 1192.4.

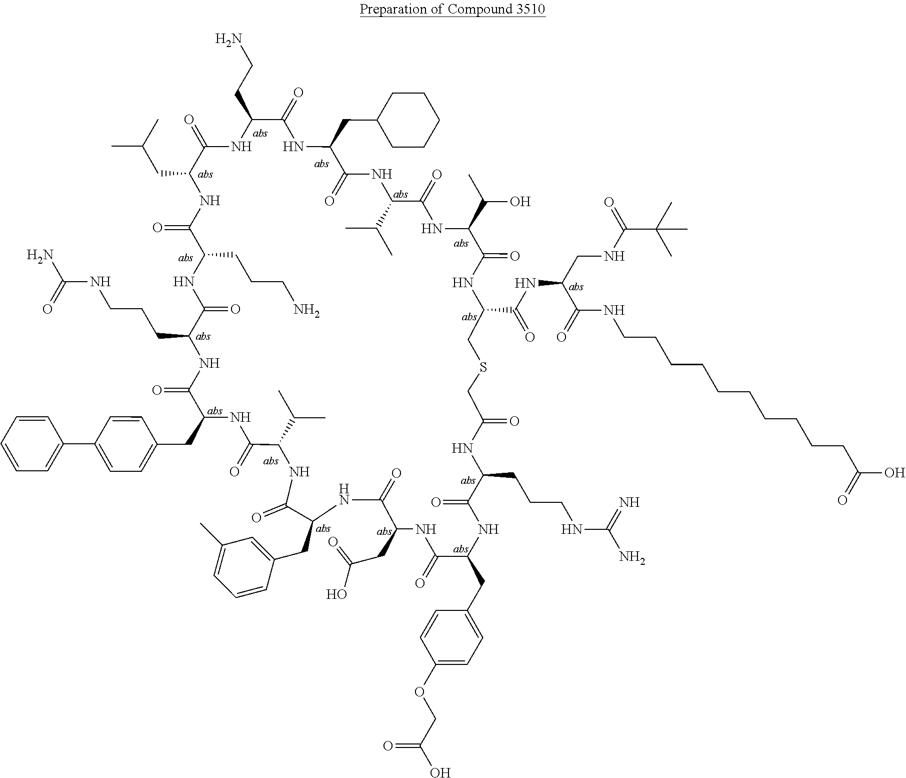 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CNC(=O)C(C)(C)C)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3510 was prepared on a 50 μmol scale. The yield of the product was 15.4 mg, and its estimated purity by LCMS analysis was 96.1%. Analysis condition A: Retention time=1.83 min; ESI-MS(+) m/z [M+2H]²⁺: 1165.1.

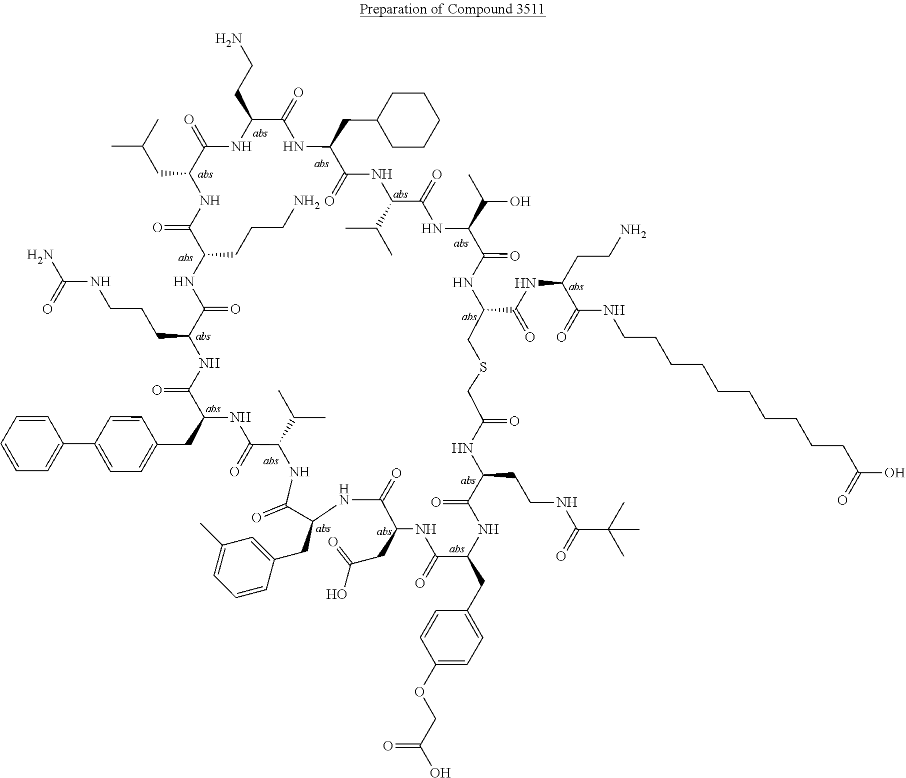 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)C(C)(C)C)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3511 was prepared on a 50 μmol scale. The yield of the product was 13 mg, and its estimated purity by LCMS analysis was 92.2%. Analysis condition B: Retention time=1.72 min; ESI-MS(+) m/z [M+2H]²⁺: 1144.

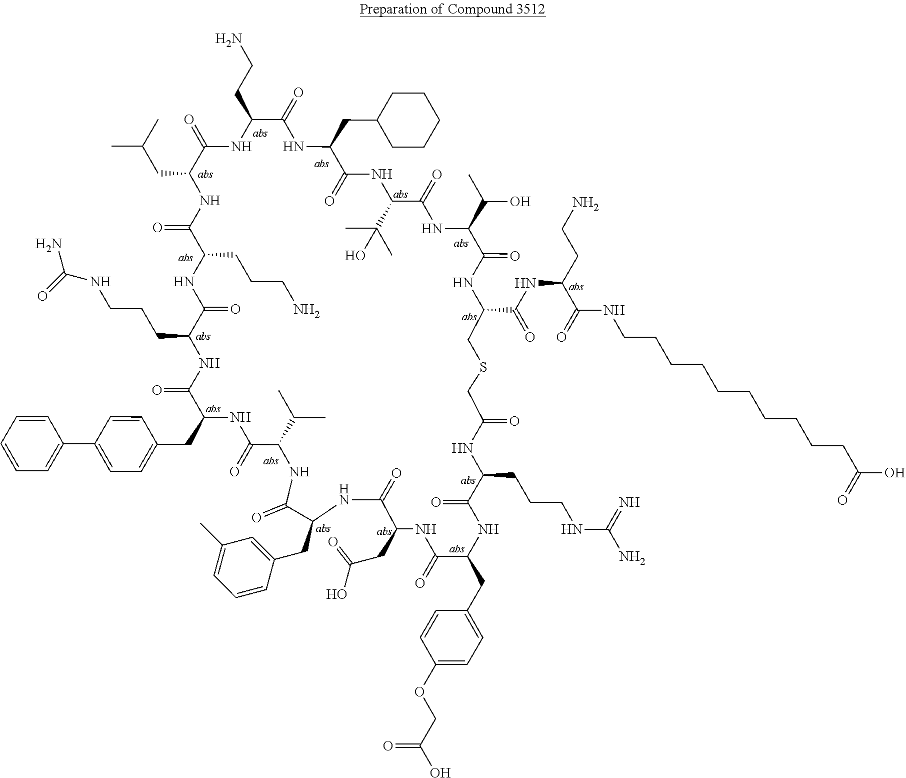 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3512 was prepared on a 50 μmol scale. The yield of the product was 11.8 mg, and its estimated purity by LCMS analysis was 97.5%. Analysis condition A: Retention time=2.36 min; ESI-MS(+) m/z [M+2H]²⁺: 1138.1.

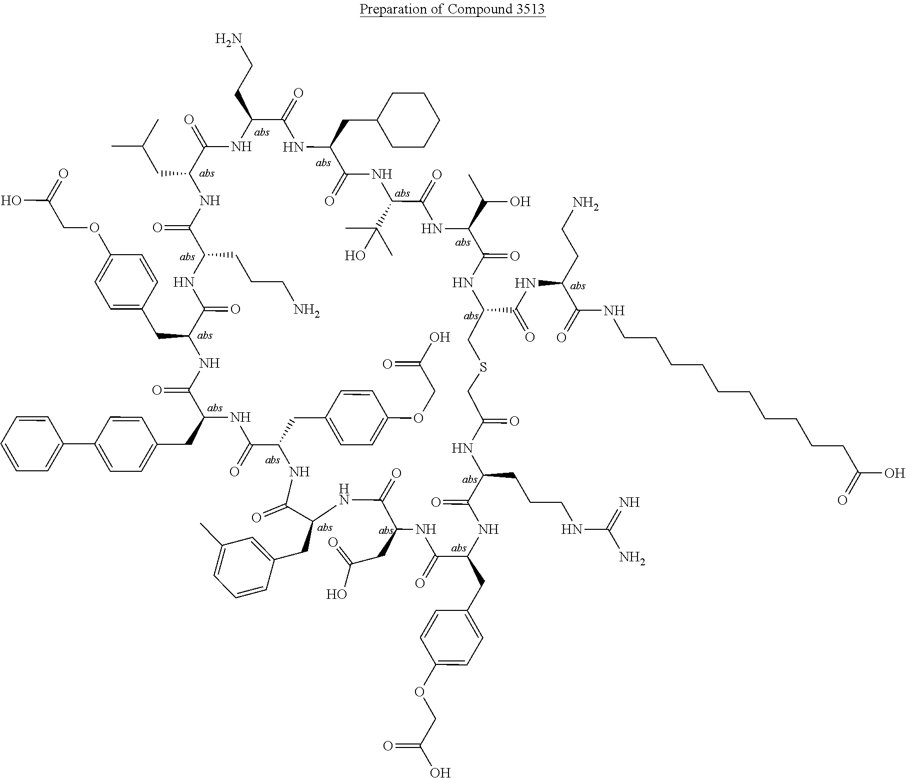 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3513 was prepared on a 50 μmol scale. The yield of the product was 3.9 mg, and its estimated purity by LCMS analysis was 98.4%. Analysis condition A: Retention time=1.63 min; ESI-MS(+) m/z [M+2H]²⁺: 1231.

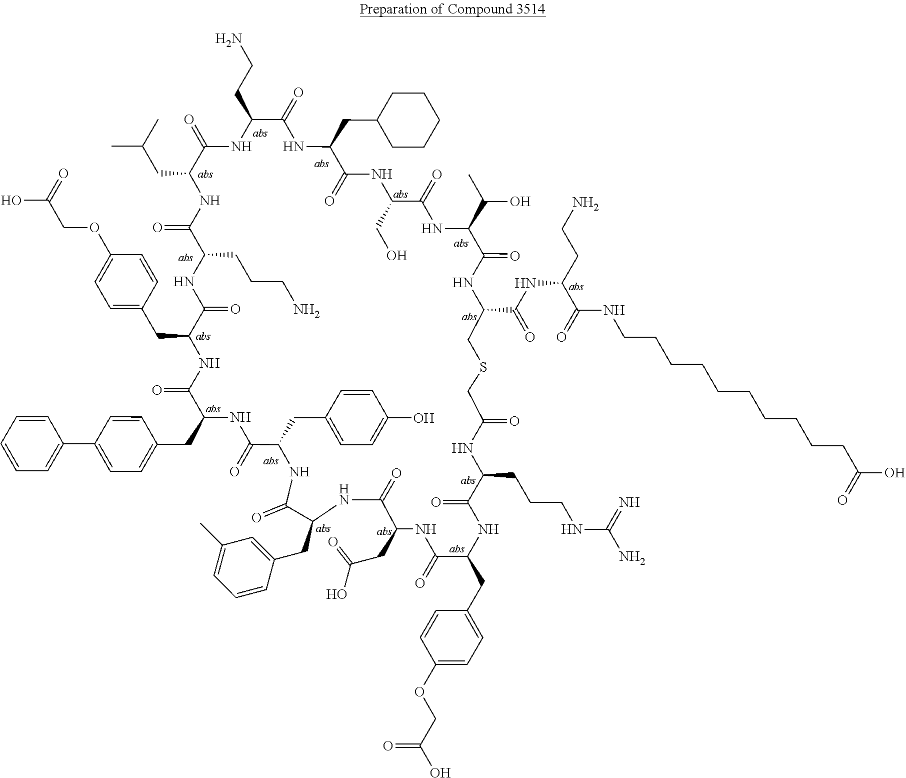 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CCN)C(=O)NCCCCCCCCCCC(=O)O)NC(=O)[C@H](C(C)O)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 3514 was prepared on a 50 μmol scale. The yield of the product was 45.2 mg, and its estimated purity by LCMS analysis was 90.7%. Analysis condition A: Retention time=1.61 min; ESI-MS(+) m/z [M+2H]²⁺: 1195.7.

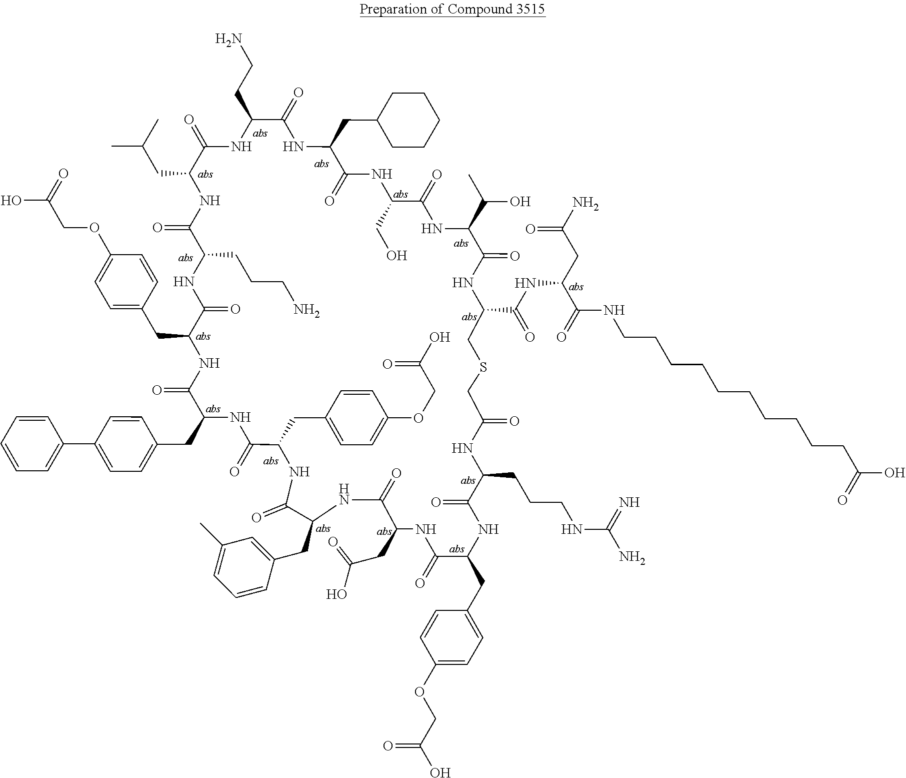 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3515 was prepared on a 50 μmol scale. The yield of the product was 15.8 mg, and its estimated purity by LCMS analysis was 100%. Analysis condition A: Retention time=1.58 min; ESI-MS(+) m/z [M+2H]²⁺: 1224.1.

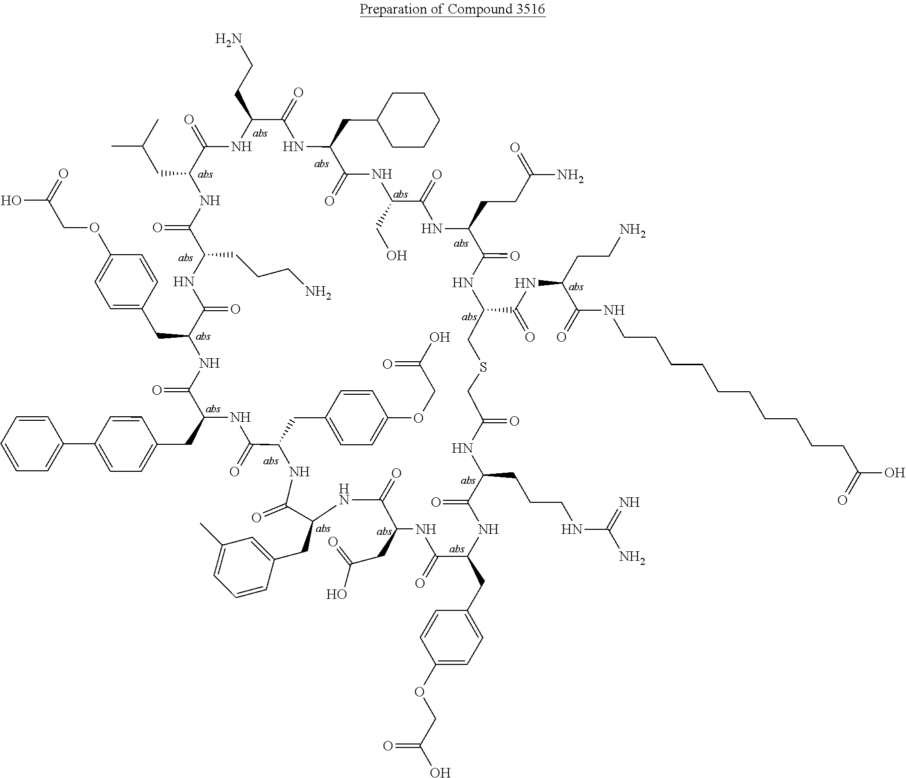 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3516 was prepared on a 50 μmol scale. The yield of the product was 9 mg, and its estimated purity by LCMS analysis was 99.3%. Analysis condition A: Retention time=1.54 min; ESI-MS(+) m/z [M+2H]²⁺: 1230.3.

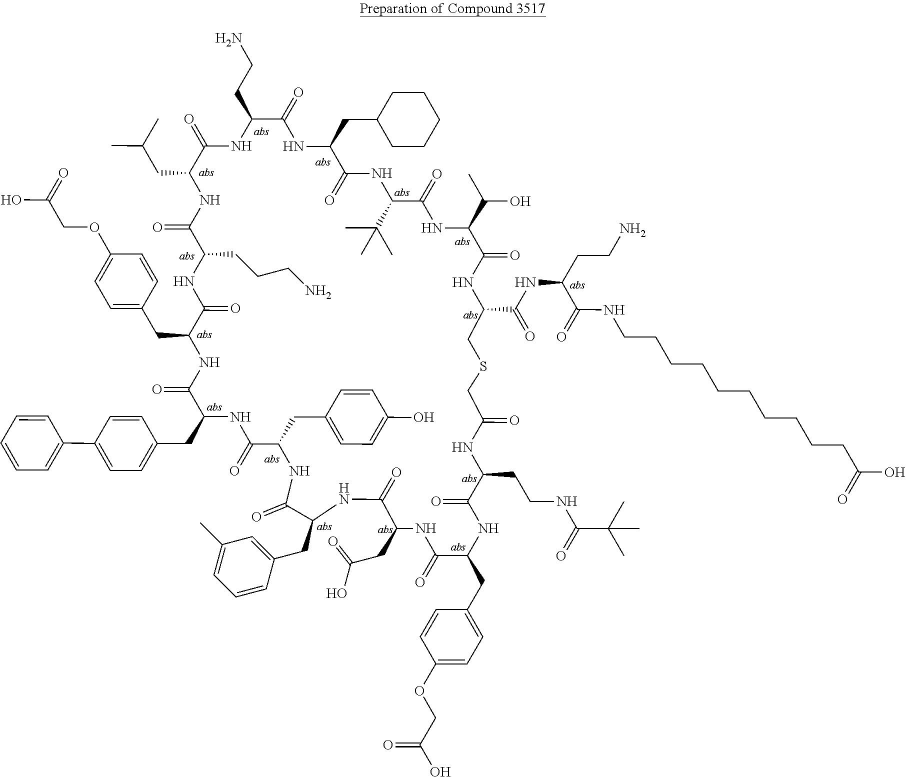 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)C(C)(C)C)NC(=O)CSC[C@@H](C(=O)N[C@@H](CCN)C(=O)NCCCCCCCCCCC(=O)O)NC(=O)[C@H](C(C)O)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 3517 was prepared on a 50 μmol scale. The yield of the product was 36.3 mg, and its estimated purity by LCMS analysis was 95.8%. Analysis condition A: Retention time=1.67, 1.71 min; ESI-MS(+) m/z [M+2H]²⁺: 1215.2, 1215.2.

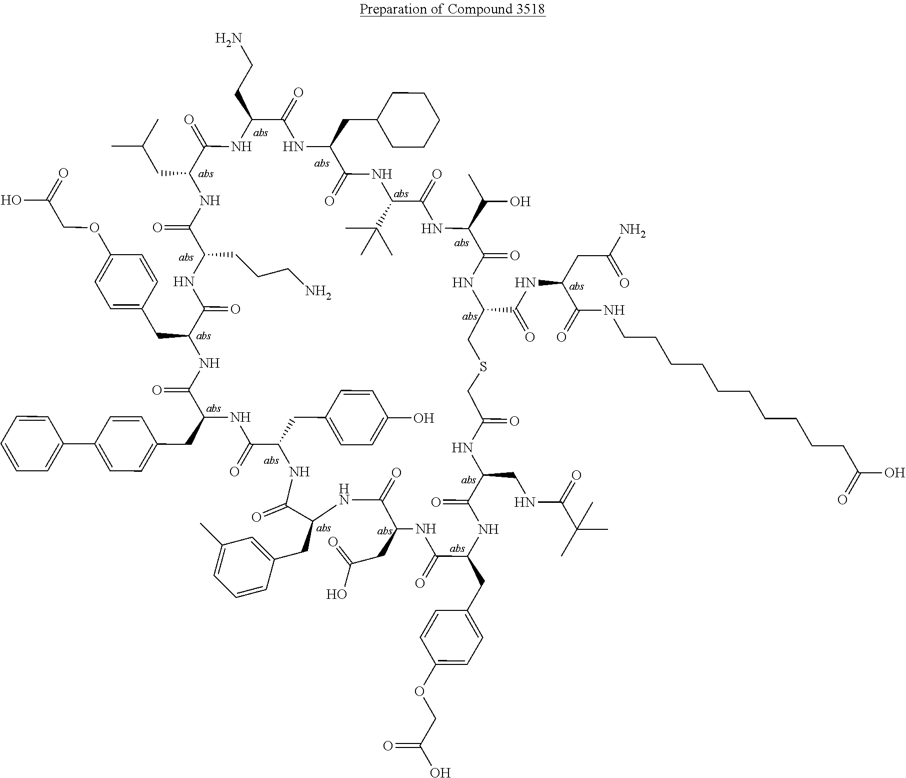 [SMILES: Cc1cccc(C[C@@H]2NC(=O)[C@H](CC(=O)O)NC(=O)[C@H](Cc3ccc(OCC(=O)O)cc3)NC(=O)[C@H](CNC(=O)C(C)(C)C)NC(=O)CSC[C@@H](C(=O)N[C@@H](CC(N)=O)C(=O)NCCCCCCCCCCC(=O)O)NC(=O)[C@H](C(C)O)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 3518 was prepared on a 50 μmol scale. The yield of the product was 8.3 mg, and its estimated purity by LCMS analysis was 95.2%. Analysis condition B: Retention time=1.84 min; ESI-MS(+) m/z [M+2H]²⁺: 1215.

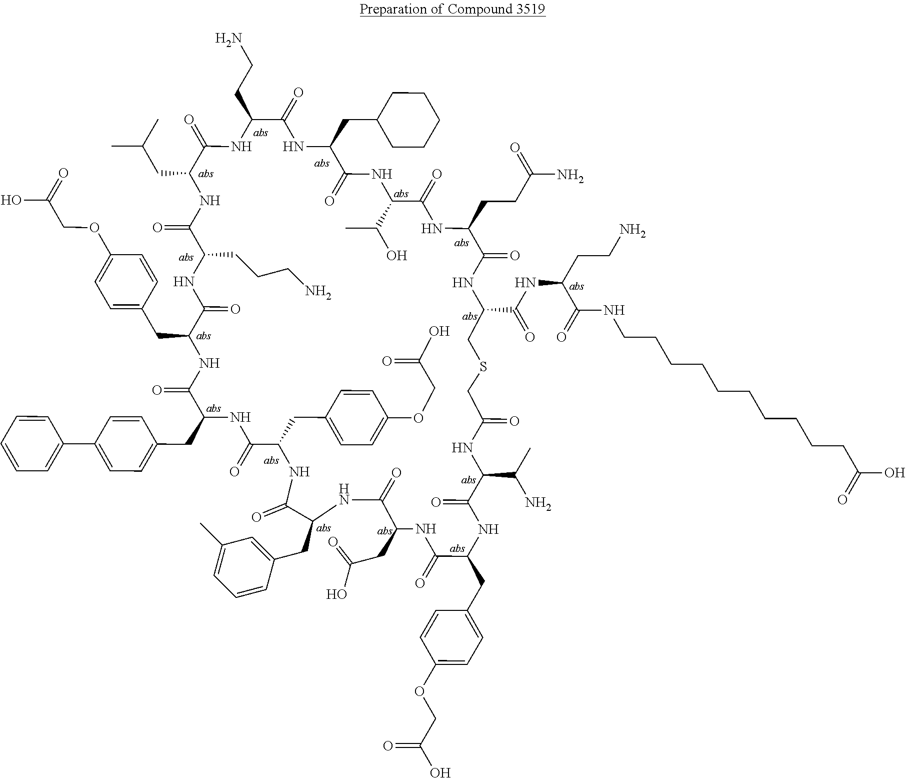 [SMILES: Cc1cccc(C[C@@H]2NC(=O)[C@H](CC(=O)O)NC(=O)[C@H](Cc3ccc(OCC(=O)O)cc3)NC(=O)[C@H](C(C)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3519 was prepared on a 50 μmol scale. The yield of the product was 45.5 mg, and its estimated purity by LCMS analysis was 97.5%. Analysis condition B: Retention time=1.63 min; ESI-MS(+) m/z [M+2H]²⁺: 1209.9.

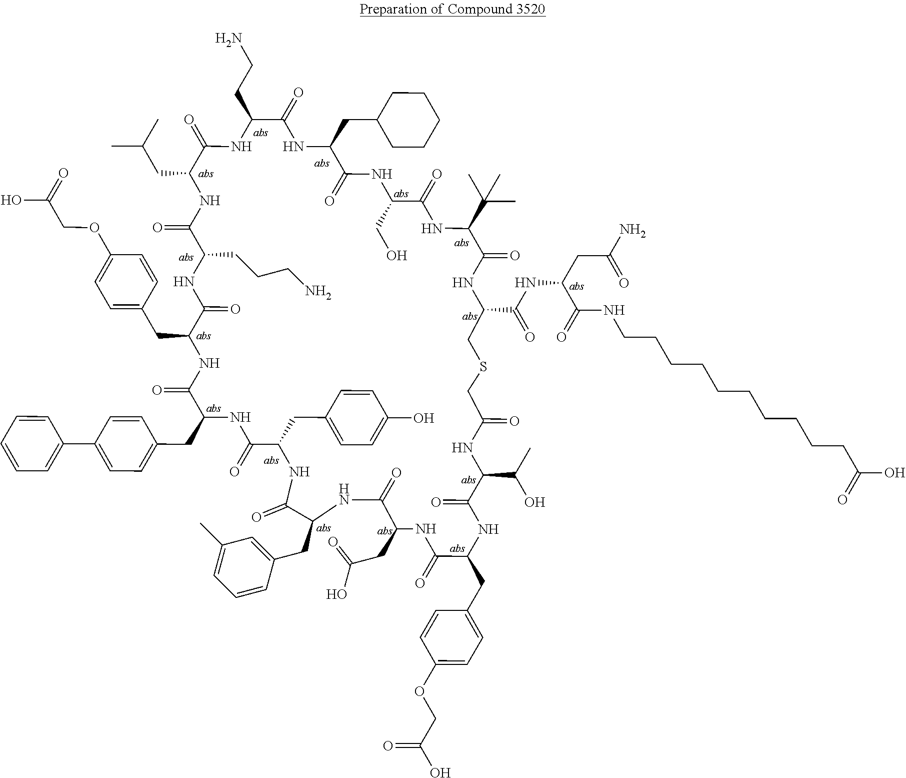 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](C(C)(C)C)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 3520 was prepared on a 50 μmol scale. The yield of the product was 19.3 mg, and its estimated purity by LCMS analysis was 98.9%. Analysis condition B: Retention time=1.9 min; ESI-MS(+) m/z [M+3H]³⁺: 783.

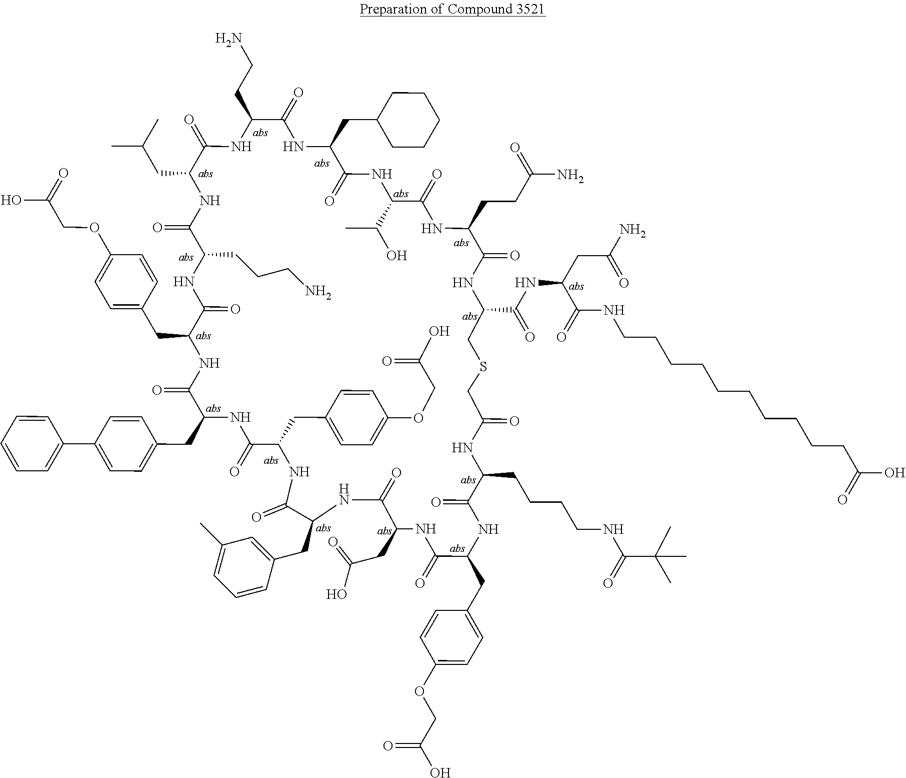 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=O)C(C)(C)C)NC(=O)CSC[C@@H](C(=O)N[C@@H](CC(N)=O)C(=O)NCCCCCCCCCCC(=O)O)NC(=O)[C@H](CCC(N)=O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3521 was prepared on a 50 μmol scale. The yield of the product was 42.1 mg, and its estimated purity by LCMS analysis was 100%. Analysis condition A: Retention time=1.44 min; ESI-MS(+) m/z [M+3H]³⁺: 846.5.

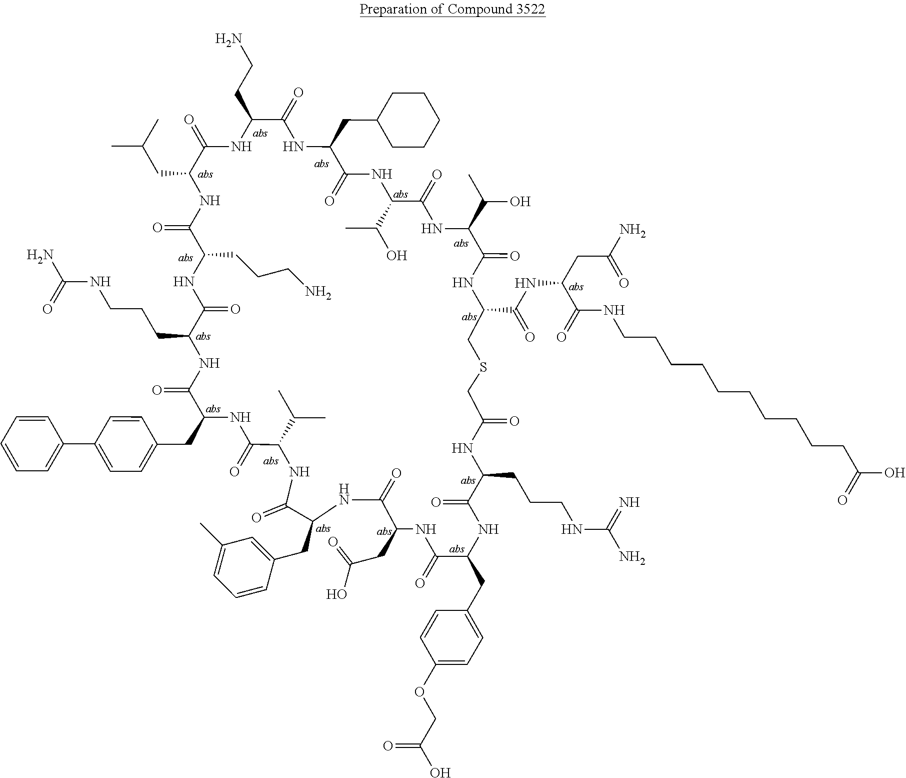 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3522 was prepared on a 50 μmol scale. The yield of the product was 32 mg, and its estimated purity by LCMS analysis was 95.5%. Analysis condition A: Retention time=1.8 min; ESI-MS(+) m/z [M+2H]²⁺: 1138.1.

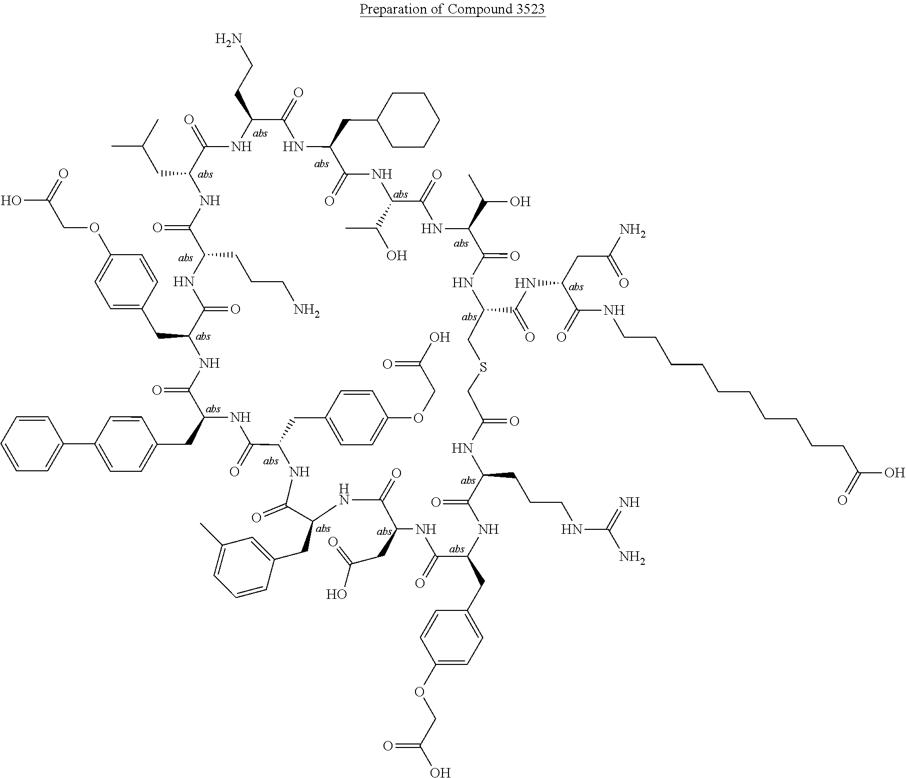 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3523 was prepared on a 50 μmol scale. The yield of the product was 14.8 mg, and its estimated purity by LCMS analysis was 95.3%. Analysis condition A: Retention time=1.45 min; ESI-MS(+) m/z [M+2H]²⁺: 1231.

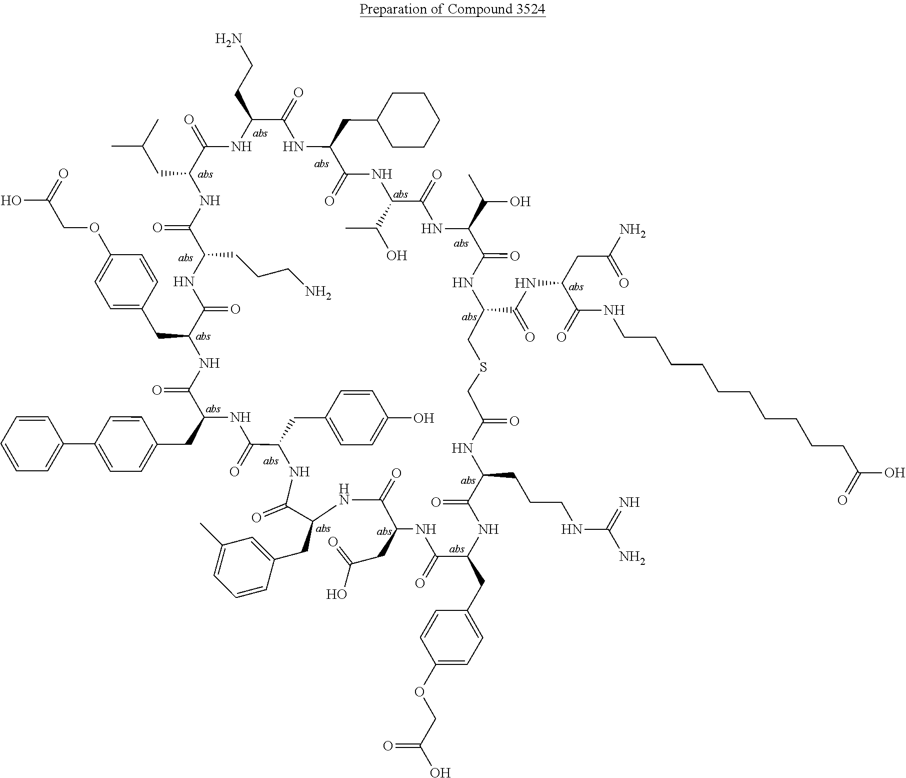 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 3524 was prepared on a 50 μmol scale. The yield of the product was 21.9 mg, and its estimated purity by LCMS analysis was 95.7%. Analysis condition B: Retention time=1.69 min; ESI-MS(+) m/z [M+2H]²⁺: 1202.

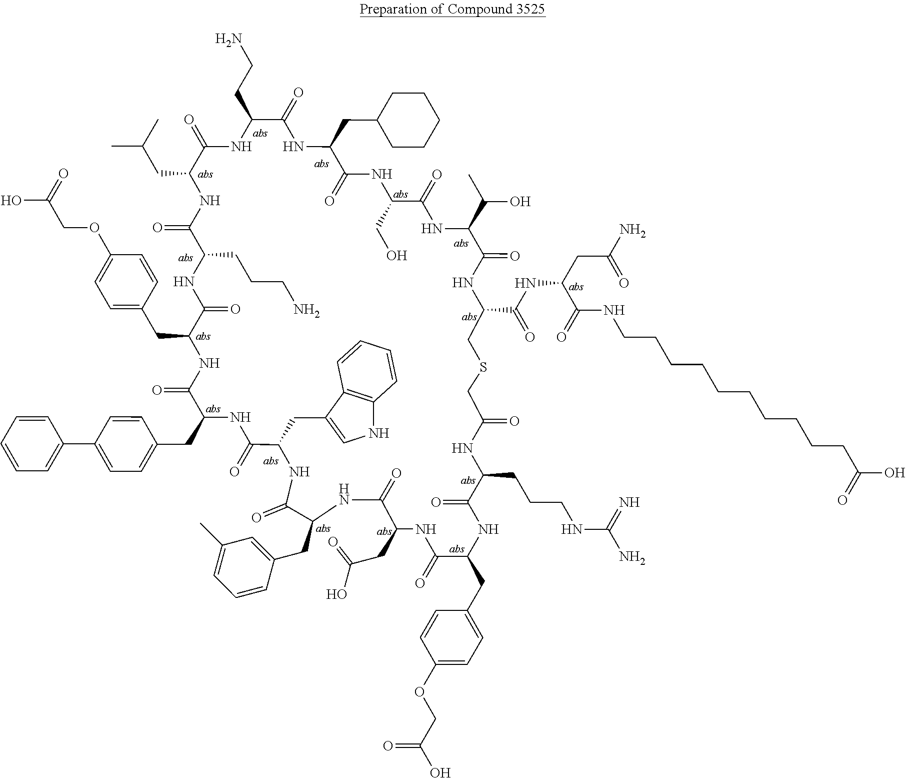 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3c[nH]c4ccccc34)NC2=O)c1]

Compound 3525 was prepared on a 50 μmol scale. The yield of the product was 29.5 mg, and its estimated purity by LCMS analysis was 98.1%. Analysis condition B: Retention time=1.8 min; ESI-MS(+) m/z [M+2H]²⁺: 1207.

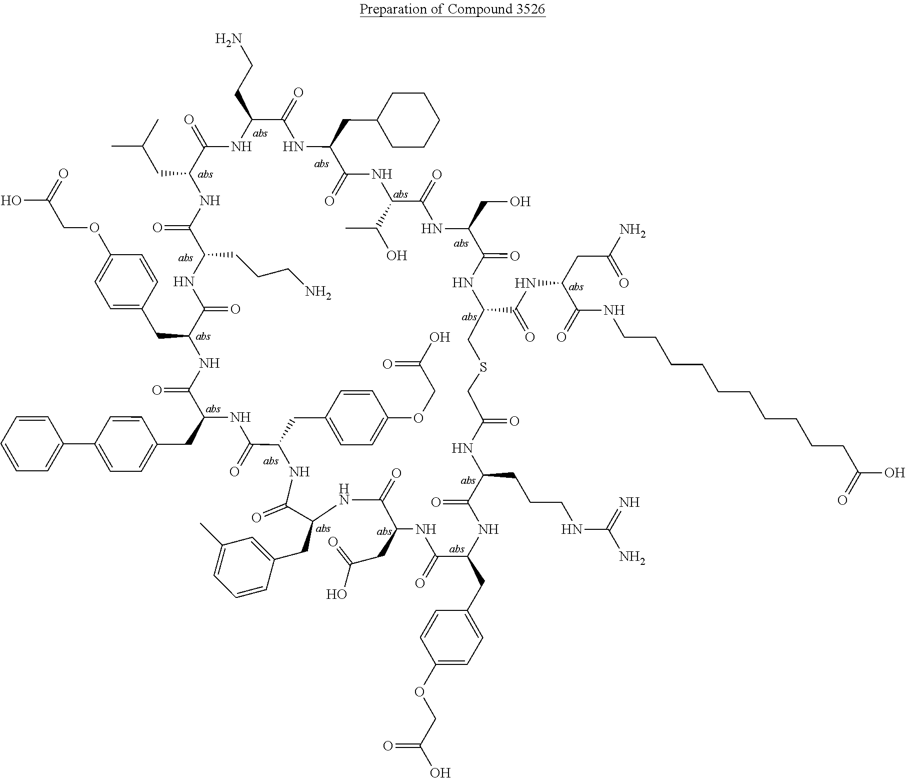 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3526 was prepared on a 50 μmol scale. The yield of the product was 11.2 mg, and its estimated purity by LCMS analysis was 99.1%. Analysis condition B: Retention time=1.68 min; ESI-MS(+) m/z [M+2H]²⁺: 1224.1.

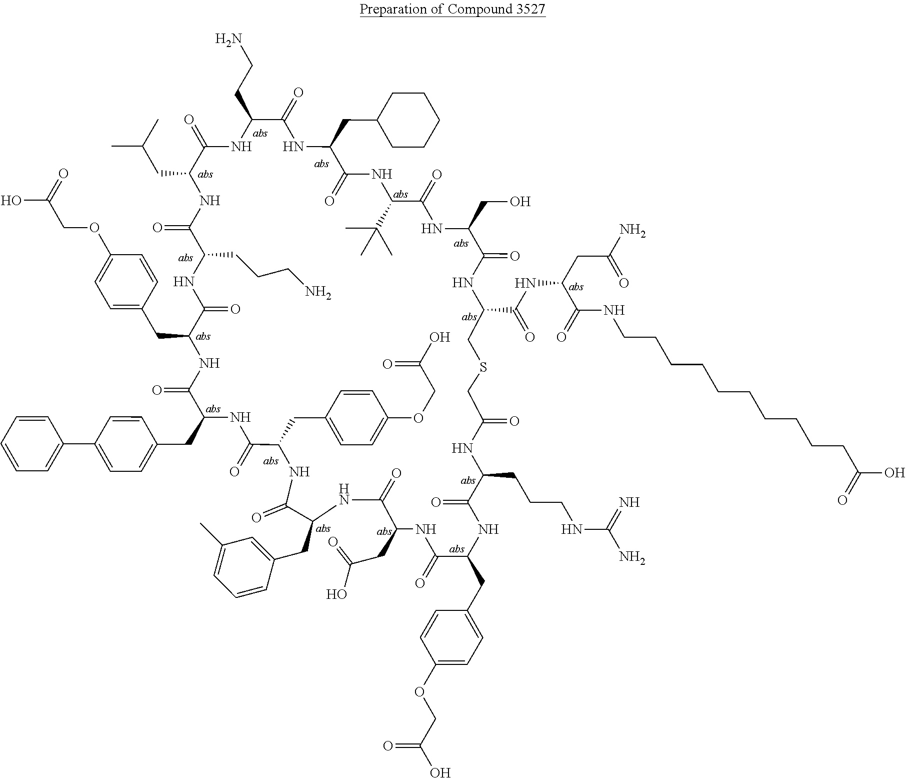 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CO)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3527 was prepared on a 50 μmol scale. The yield of the product was 11.3 mg, and its estimated purity by LCMS analysis was 95.4%. Analysis condition A: Retention time=1.55 min; ESI-MS(+) m/z [M+2H]²⁺: 1230.

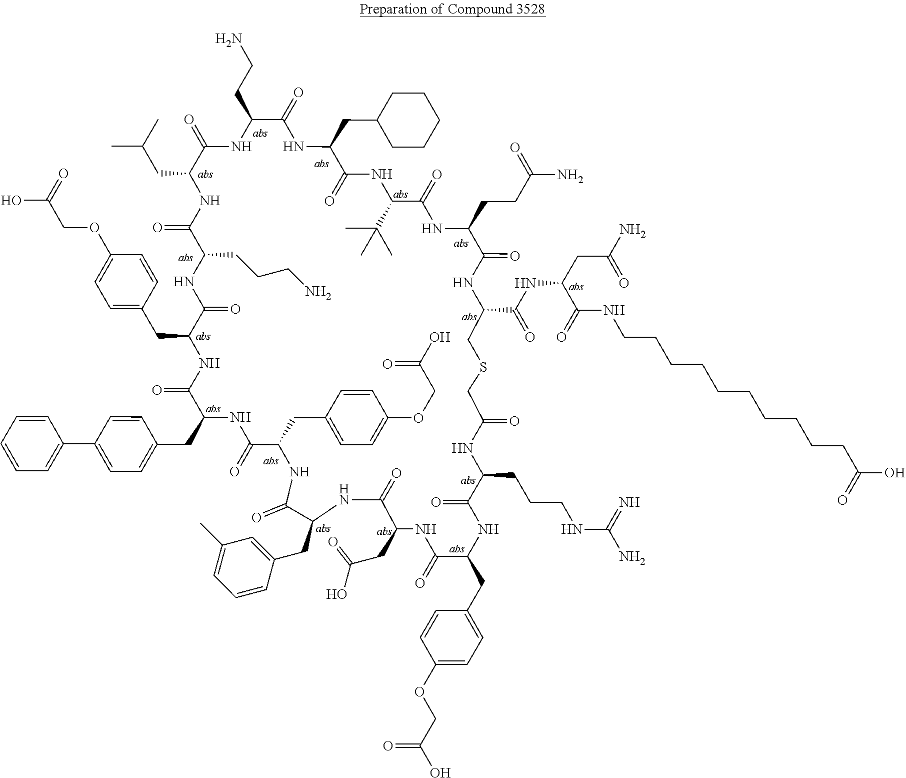 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCC(N)=O)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3528 was prepared on a 50 μmol scale. The yield of the product was 15.2 mg, and its estimated purity by LCMS analysis was 100%. Analysis condition A: Retention time=1.5 min; ESI-MS(+) m/z [M+2H]²⁺: 1250.4.

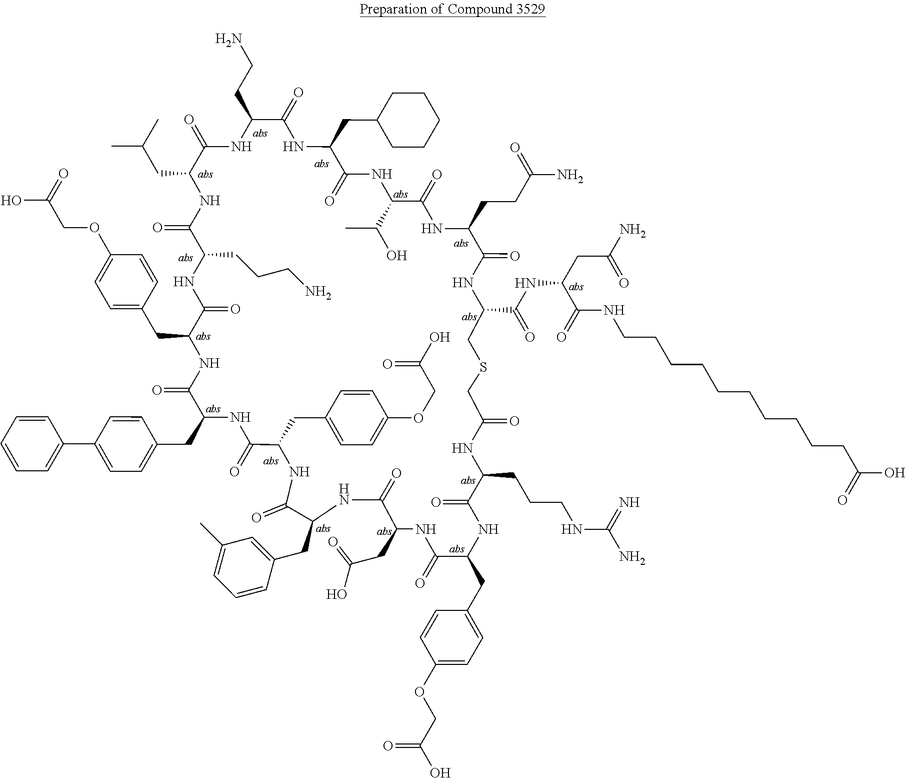 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](CCC(N)=O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3529 was prepared on a 50 μmol scale. The yield of the product was 27.1 mg, and its estimated purity by LCMS analysis was 95.7%. Analysis condition A: Retention time=1.35 min; ESI-MS(+) m/z [M+2H]²⁺: 1245.

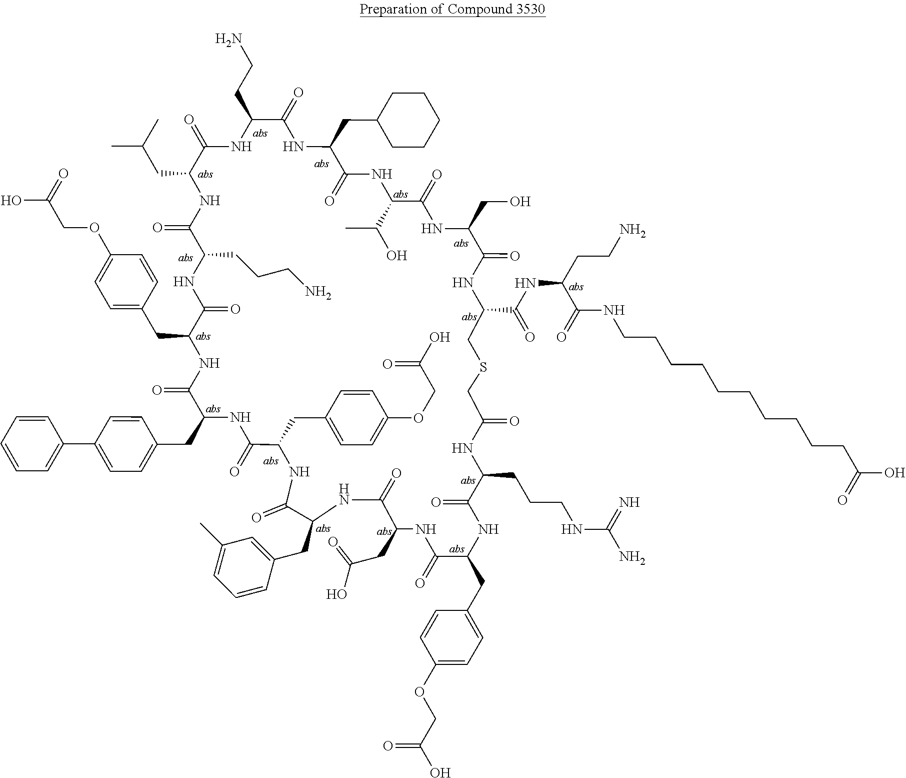 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3530 was prepared on a 50 μmol scale. The yield of the product was 13.4 mg, and its estimated purity by LCMS analysis was 95%. Analysis condition A: Retention time=1.51 min; ESI-MS(+) m/z [M+2H]²⁺: 1217.1.

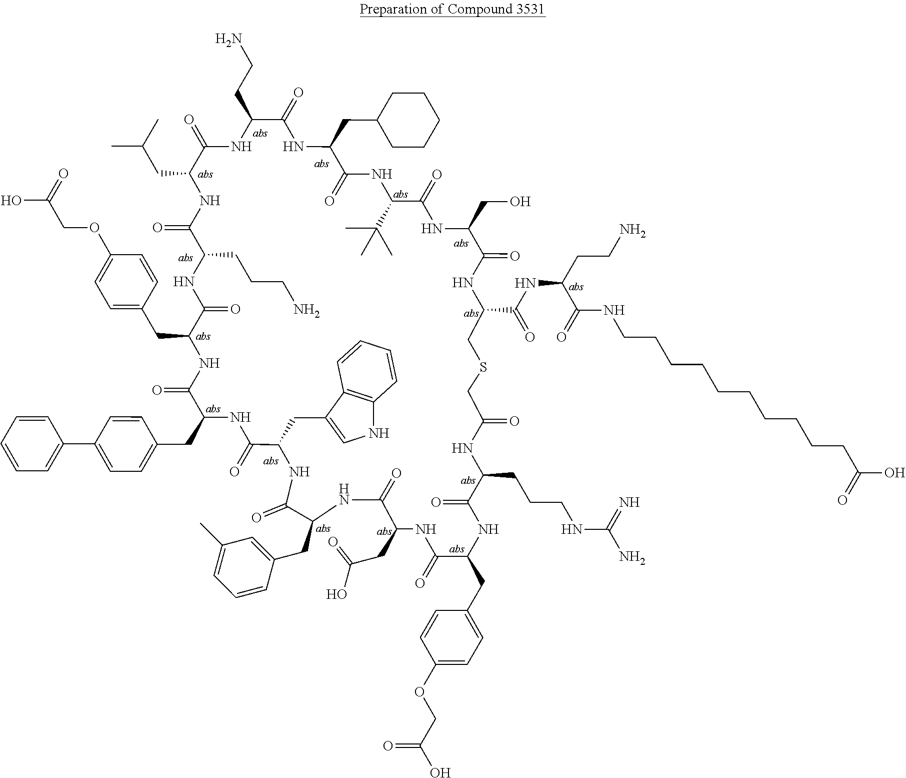 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3c[nH]c4ccccc34)NC2=O)c1]

Compound 3531 was prepared on a 50 μmol scale. The yield of the product was 23.9 mg, and its estimated purity by LCMS analysis was 92.7%. Analysis condition A: Retention time=1.64 min; ESI-MS(+) m/z [M+3H]³⁺: 804.3.

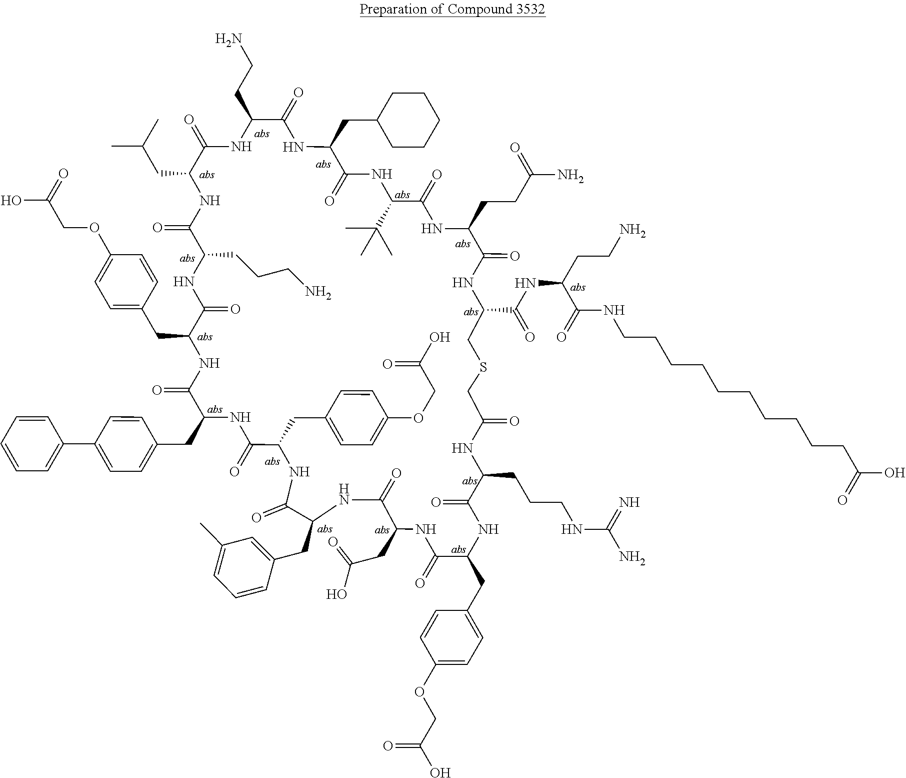 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3532 was prepared on a 50 μmol scale. The yield of the product was 66.8 mg, and its estimated purity by LCMS analysis was 96.4%. Analysis condition A: Retention time=1.49 min; ESI-MS(+) m/z [M+2H]²⁺: 1243.8.

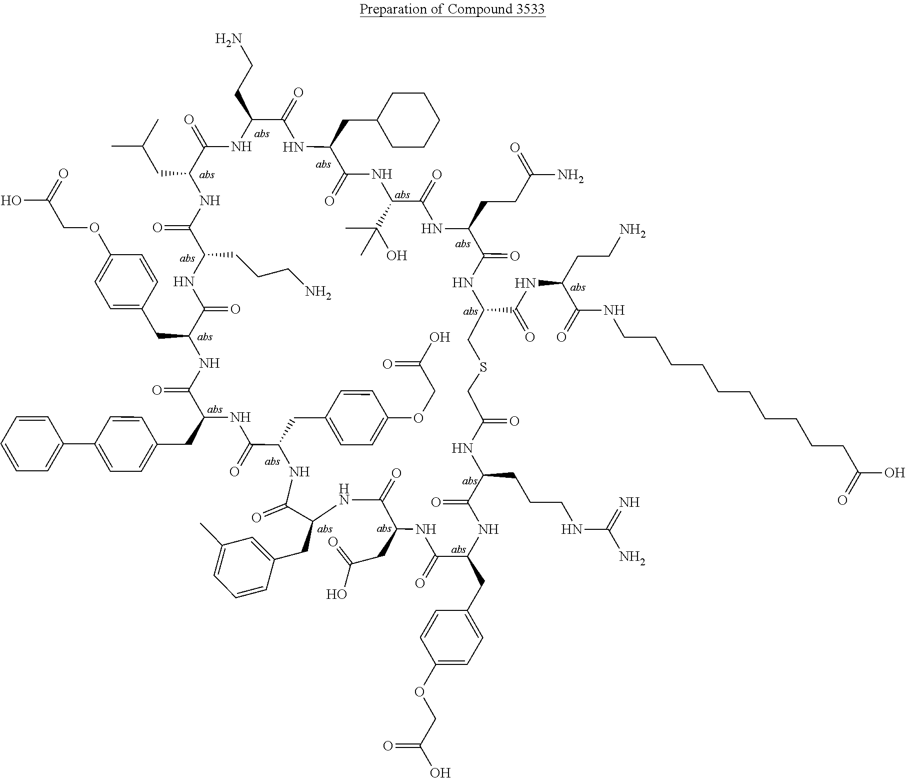 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](CCC(N)=O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3533 was prepared on a 50 μmol scale. The yield of the product was 6.6 mg, and its estimated purity by LCMS analysis was 92.4%. Analysis condition A: Retention time=1.52, 1.58 min; ESI-MS(+) m/z [M+3H]³⁺: 830.22, 830.22.

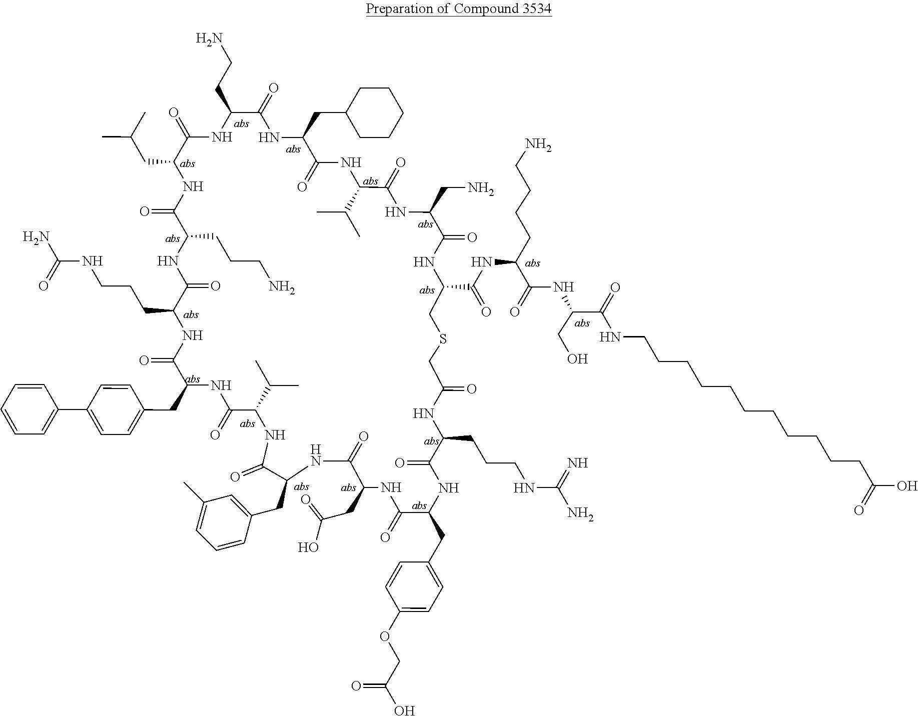 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)N[C@@H](CO)C(=O)NCCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3534 was prepared on a 50 μmol scale. The yield of the product was 24 mg, and its estimated purity by LCMS analysis was 97.1%. Analysis condition A: Retention time=1.64 min; ESI-MS(+) m/z [M+2H]²⁺: 1187.

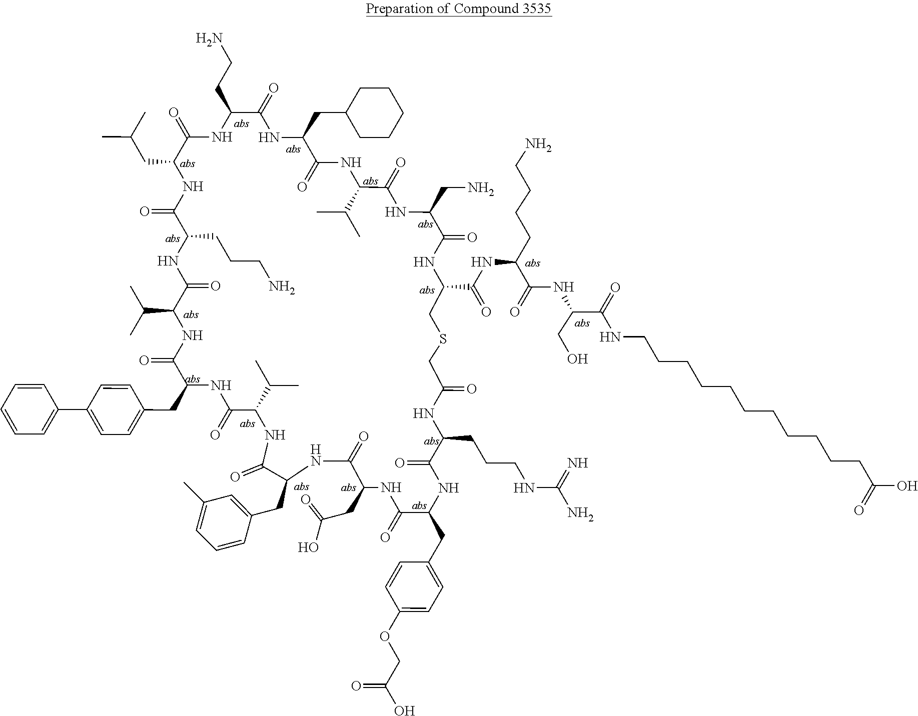 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)N[C@@H](CO)C(=O)NCCCCCCCCCCCC(=O)O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3535 was prepared on a 50 μmol scale. The yield of the product was 16.7 mg, and its estimated purity by LCMS analysis was 96%. Analysis condition A: Retention time=1.68 min; ESI-MS(+) m/z [M+3H]³⁺: 772.2.

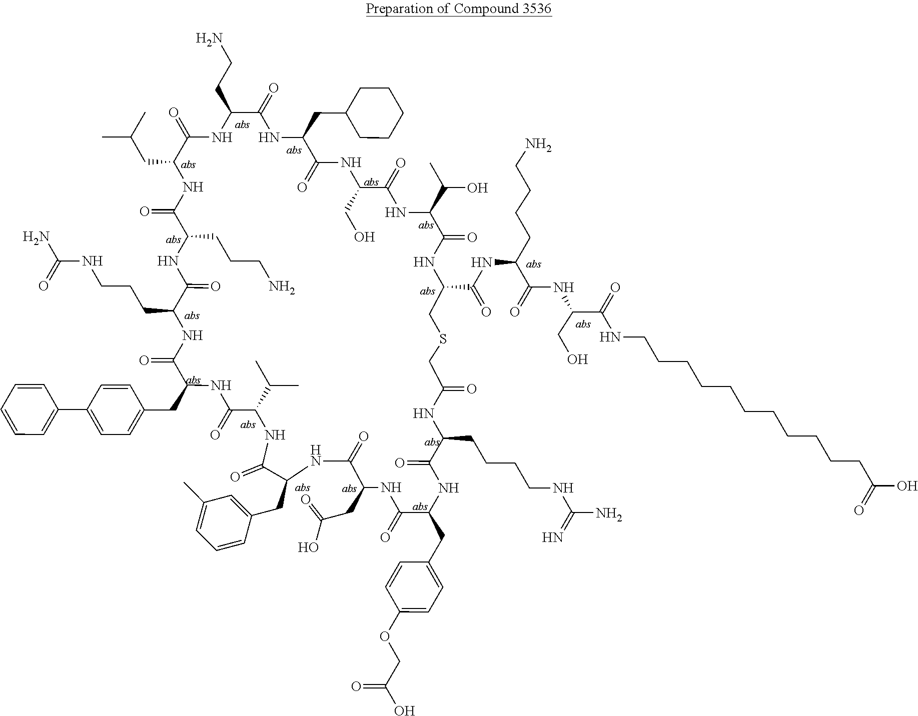 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCCN)C(=O)N[C@@H](CO)C(=O)NCCCCCCCCCCCC(=O)O)NC(=O)[C@H](C(C)O)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3536 was prepared on a 50 μmol scale. The yield of the product was 18.6 mg, and its estimated purity by LCMS analysis was 98.1%. Analysis condition A: Retention time=1.6, 1.66 min; ESI-MS(+) m/z [M+3H]³⁺: 787.2, 787.2.

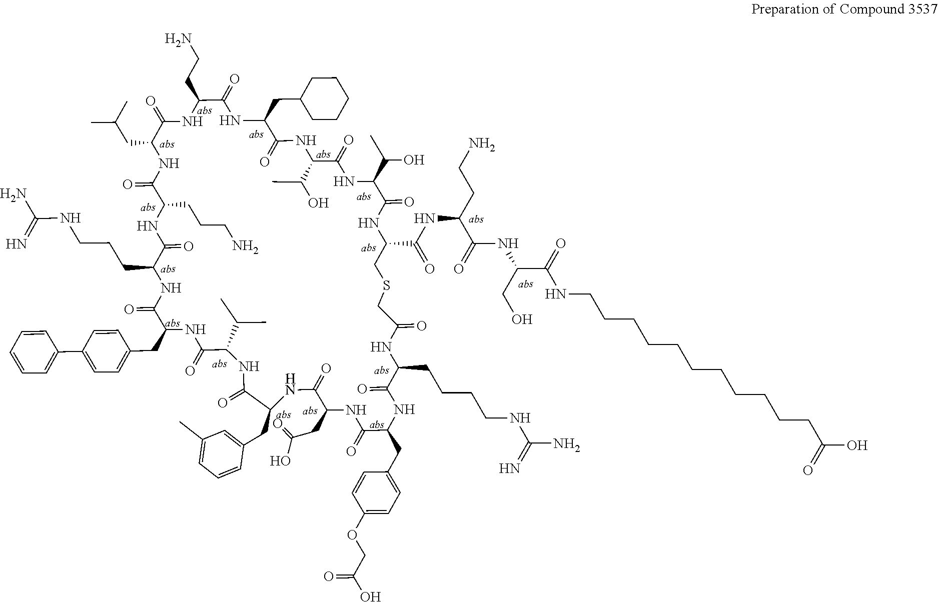 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)N[C@@H](CO)C(=O)NCCCCCCCCCCCC(=O)O)NC(=O)[C@H](C(C)O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 3537 was prepared on a 50 μmol scale. The yield of the product was 20.2 mg, and its estimated purity by LCMS analysis was 100%. Analysis condition B: Retention time=1.51 min; ESI-MS(+) m/z [M+3H]³⁺: 792.1.

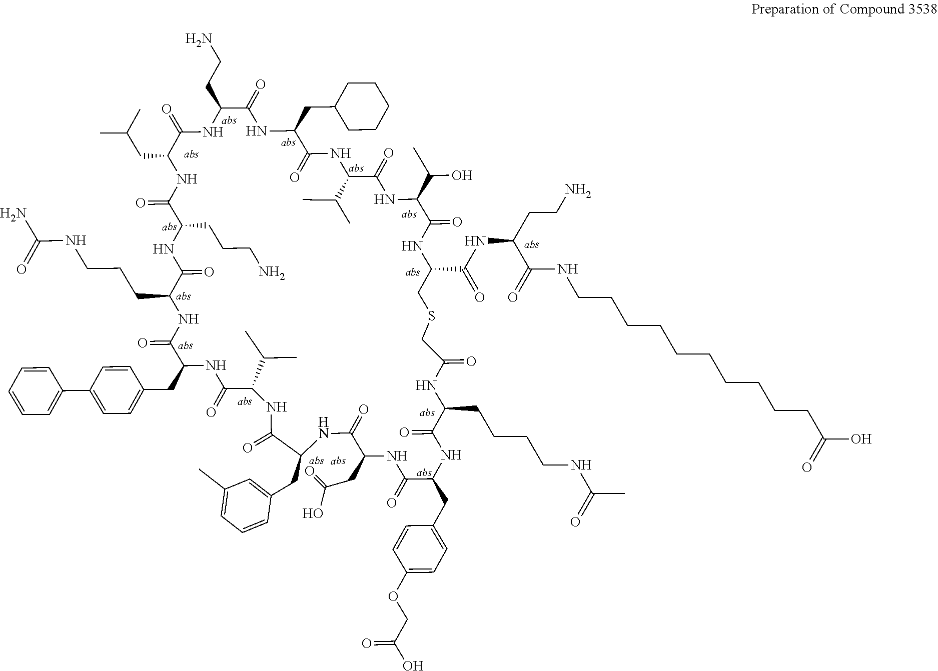 [SMILES: CC(=O)NCCCC[C@@H]1NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC1=O]

Compound 3538 was prepared on a 50 μmol scale. The yield of the product was 4.2 mg, and its estimated purity by LCMS analysis was 100%. Analysis condition A: Retention time=1.56 min; ESI-MS(+) m/z [M+2H]²⁺: 1137.

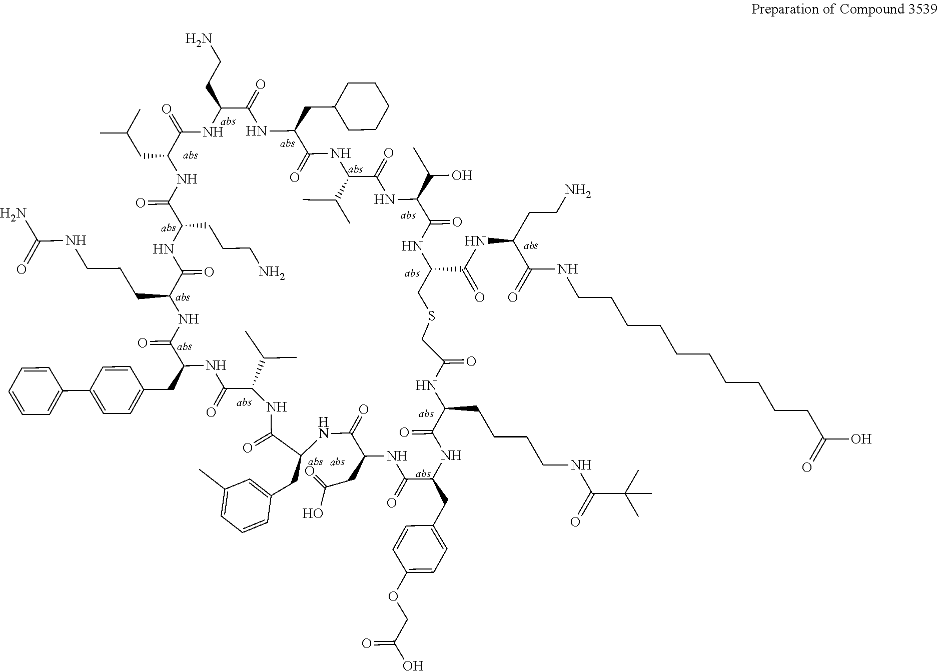 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=O)C(C)(C)C)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3539 was prepared on a 50 μmol scale. The yield of the product was 2.1 mg, and its estimated purity by LCMS analysis was 95.1%. Analysis condition A: Retention time=1.68 min; ESI-MS(+) m/z [M+2H]²⁺: 1158.2.

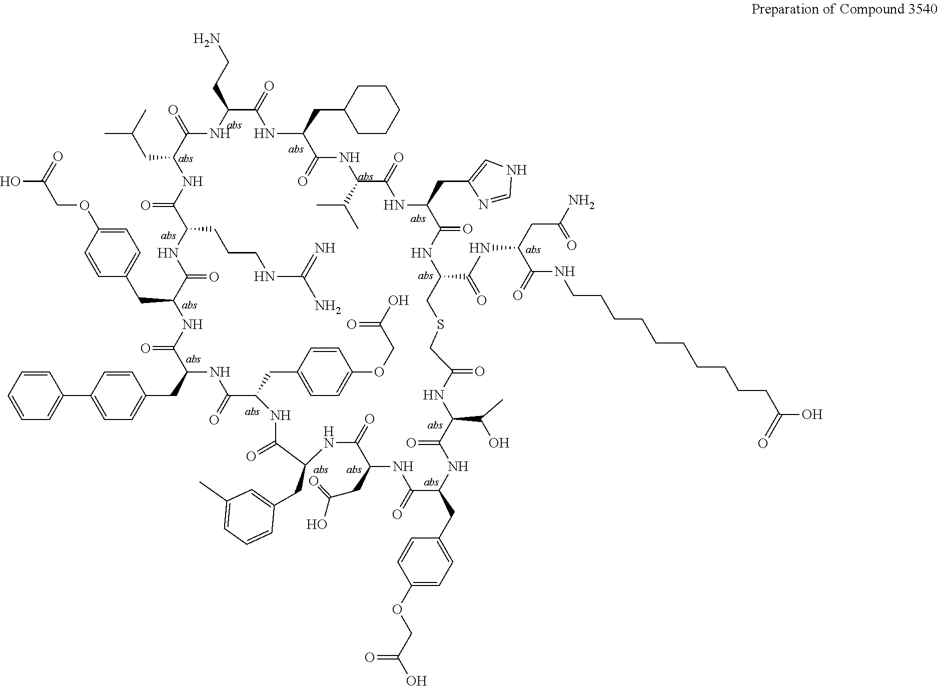 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(=O)NCCCCCCCCCCC(=O)O)NC(=O)[C@H](Cc3c[nH]cn3)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(=N)N)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3540 was prepared on a 50 μmol scale. The yield of the product was 52.7 mg, and its estimated purity by LCMS analysis was 100%. Analysis condition B: Retention time=1.63 min; ESI-MS(+) m/z [M+3H]³⁺: 814.1.

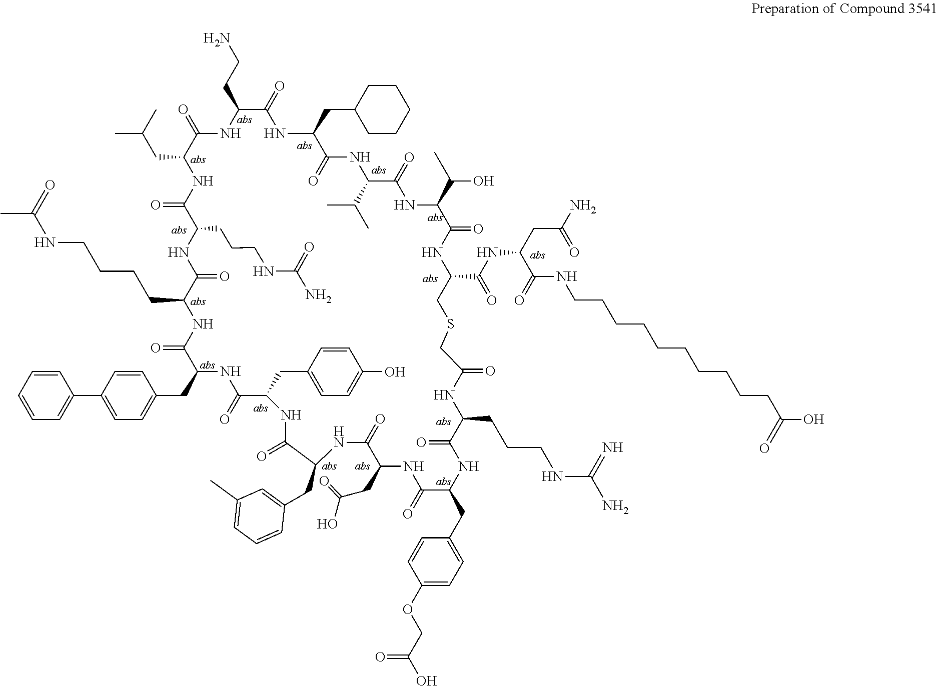 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCNC(N)=O)NC1=O]

Compound 3541 was prepared on a 50 μmol scale. The yield of the product was 20.1 mg, and its estimated purity by LCMS analysis was 98.7%. Analysis condition A: Retention time=1.61 min; ESI-MS(+) m/z [M+2H]²⁺: 1197.2.

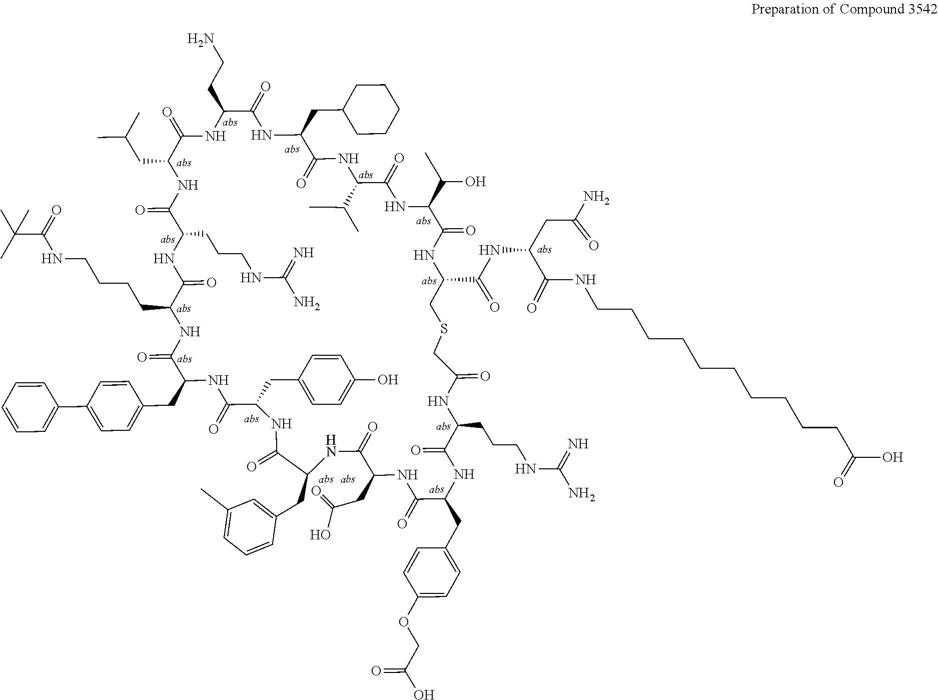 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(=N)N)NC(=O)[C@H](CCCCNC(=O)C(C)(C)C)NC(=O)[C@H](Cc3ccc(-c4ccccc4)cc3)NC(=O)[C@H](Cc3ccc(O)cc3)NC2=O)c1]

Compound 3542 was prepared on a 50 μmol scale. The yield of the product was 20.9 mg, and its estimated purity by LCMS analysis was 96%. Analysis condition B: Retention time=1.91 min; ESI-MS(+) m/z [M+3H]³⁺: 812.

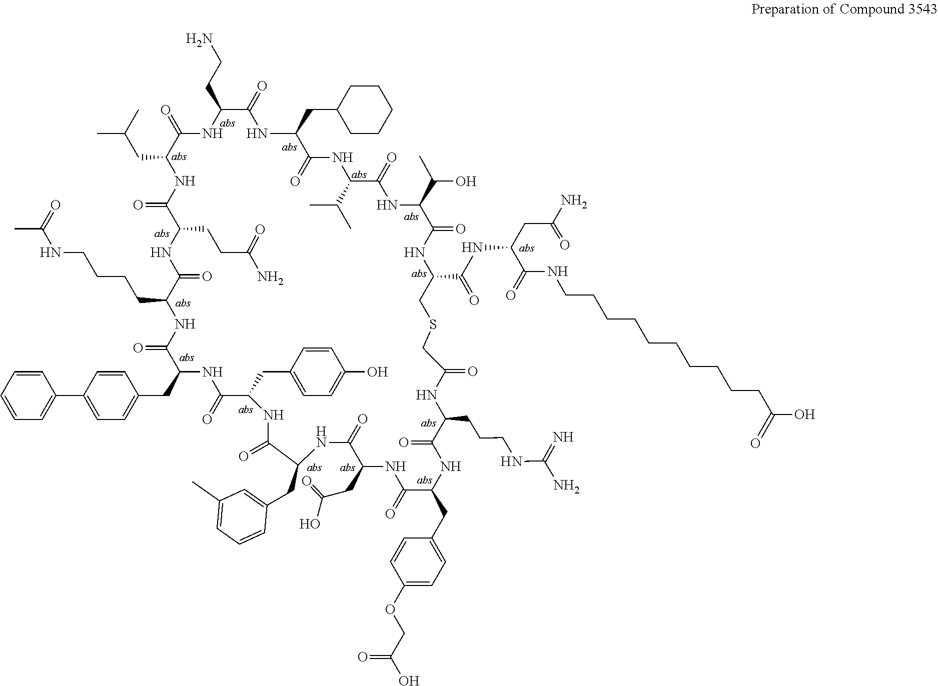 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCC(N)=O)NC1=O]

Compound 3543 was prepared on a 50 μmol scale. The yield of the product was mg, and its estimated purity by LCMS analysis was 95.3%. Analysis condition A: Retention time=1.75 min; ESI-MS(+) m/z [M+2H]²⁺: 1182.0.

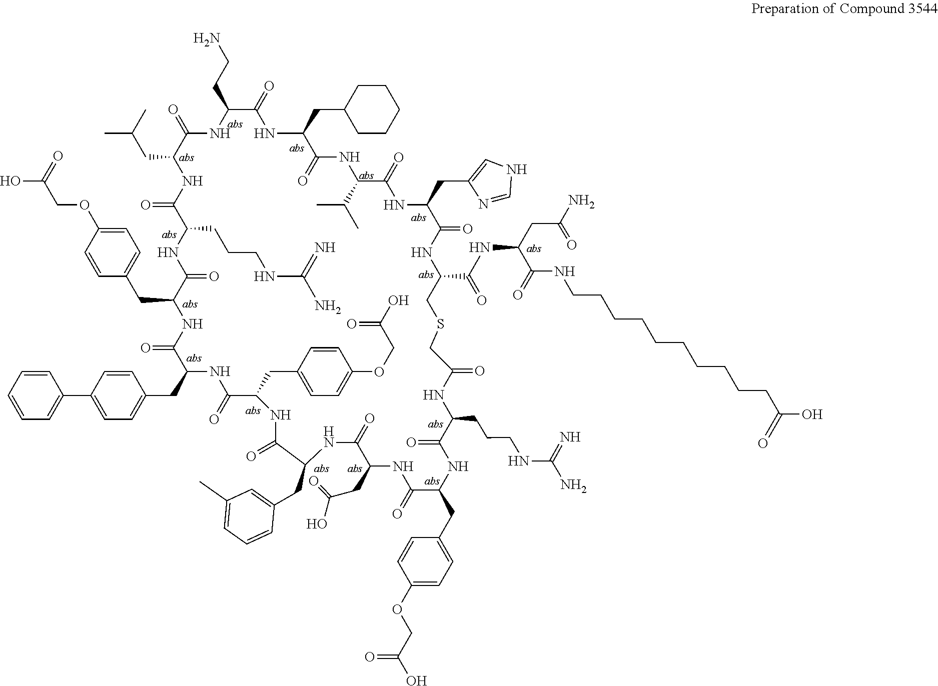 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CC(N)=O)C(=O)NCCCCCCCCCCC(=O)O)NC(=O)[C@H](Cc3c[nH]cn3)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(=N)N)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3544 was prepared on a 50 μmol scale. The yield of the product was 19.2 mg, and its estimated purity by LCMS analysis was 100%. Analysis condition A: Retention time=1.45 min; ESI-MS(+) m/z [M+2H]²⁺: 1240.8.

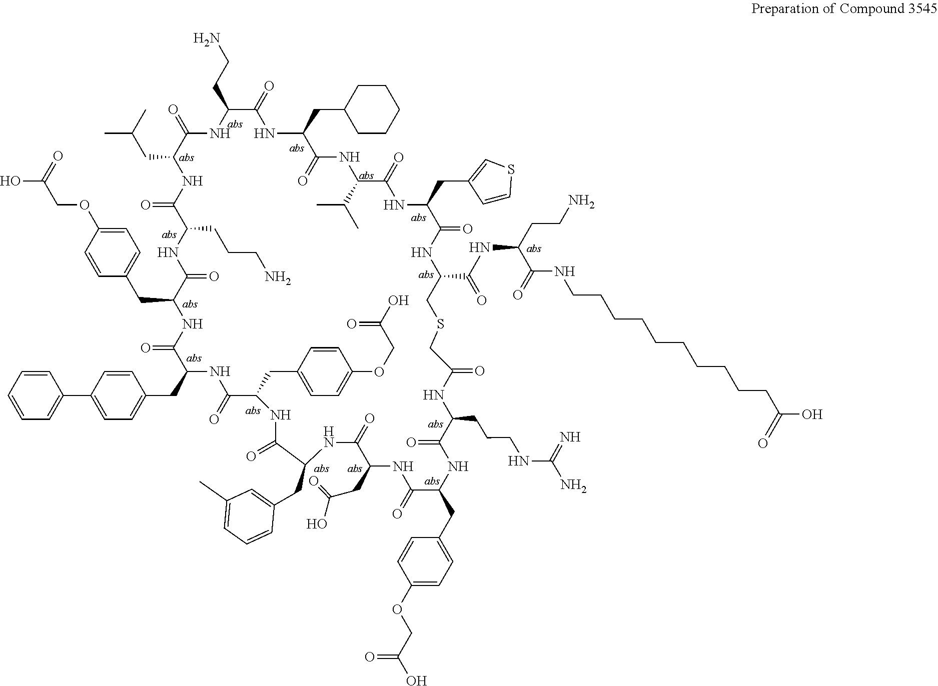 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](Cc3ccsc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3545 was prepared on a 50 μmol scale. The yield of the product was 1.4 mg, and its estimated purity by LCMS analysis was 100%. Analysis condition A: Retention time=1.61 min; ESI-MS(+) m/z [M+3H]³⁺: 833.1.

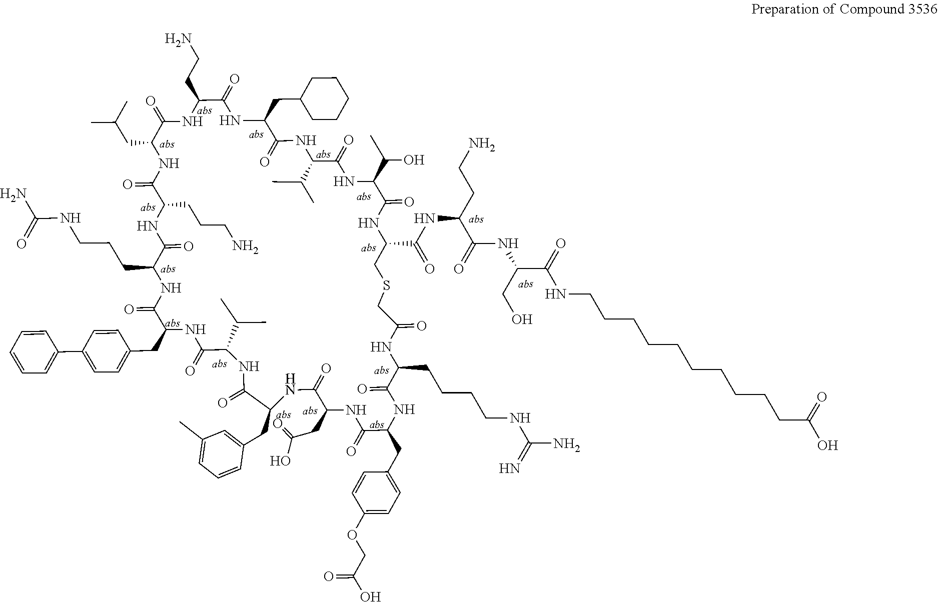 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)N[C@@H](CO)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3546 was prepared on a 50 μmol scale. The yield of the product was 5.2 mg, and its estimated purity by LCMS analysis was 97.3%. Analysis condition A: Retention time=1.58 min; ESI-MS(+) m/z [M+3H]³⁺: 791.1.

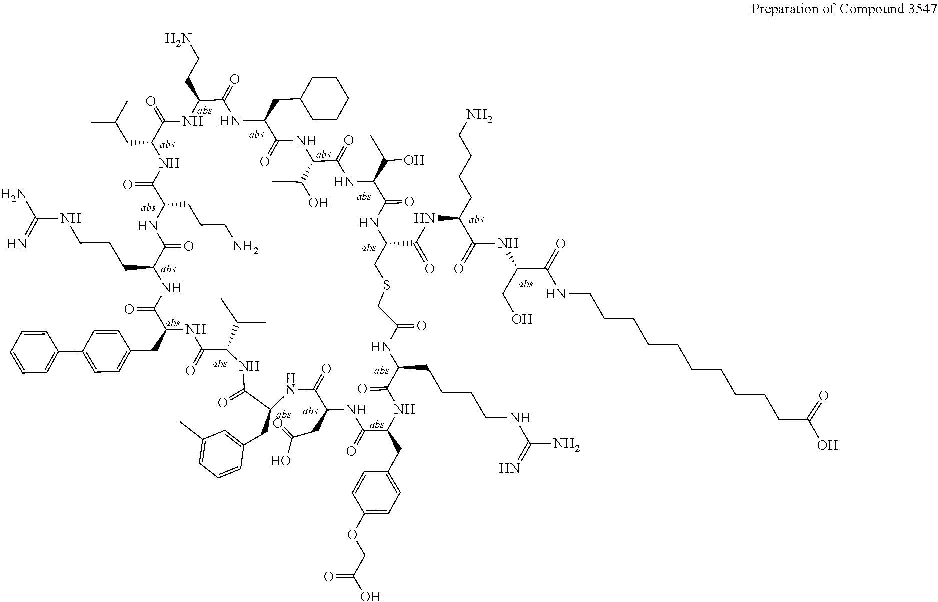 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCCN)C(=O)N[C@@H](CO)C(=O)NCCCCCCCCCCC(=O)O)NC(=O)[C@H](C(C)O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 3547 was prepared on a 50 μmol scale. The yield of the product was 5.7 mg, and its estimated purity by LCMS analysis was 100%. Analysis condition A: Retention time=1.47 min; ESI-MS(+) m/z [M+2H]²⁺: 1194.3.

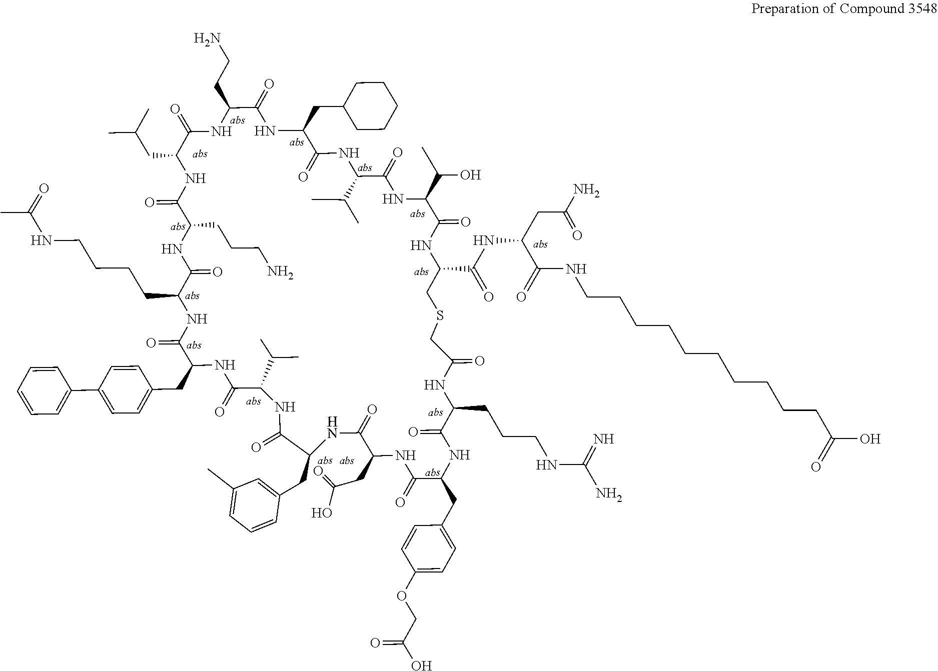 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CC(N)=O)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3548 was prepared on a 50 μmol scale. The yield of the product was 17 mg, and its estimated purity by LCMS analysis was 100%. Analysis condition A: Retention time=1.74 min; ESI-MS(+) m/z [M+2H]²⁺: 1143.

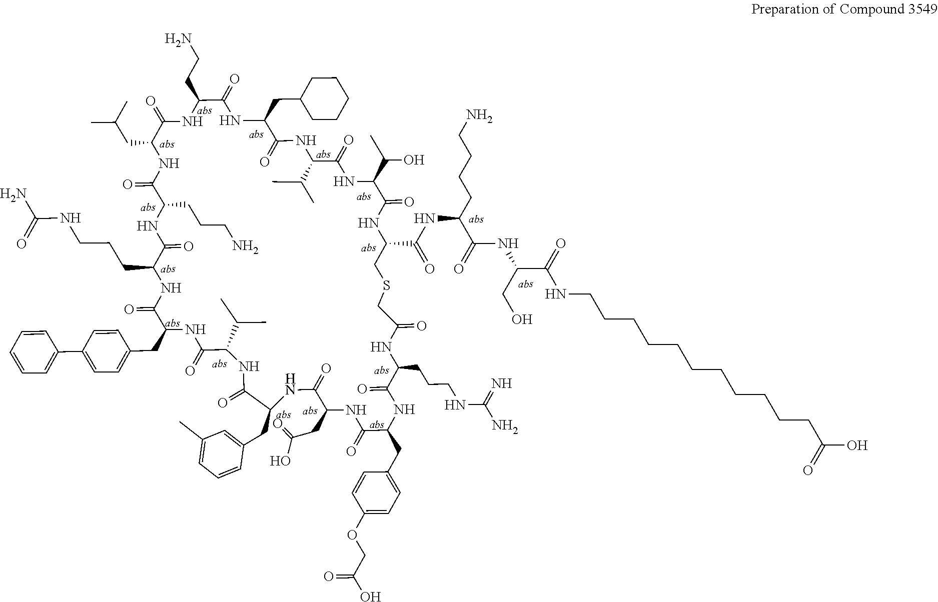 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3549 was prepared on a 50 μmol scale. The yield of the product was 14.7 mg, and its estimated purity by LCMS analysis was 100%. Analysis condition A: Retention time=1.52 min; ESI-MS(+) m/z [M+3H]³⁺: 796.1.

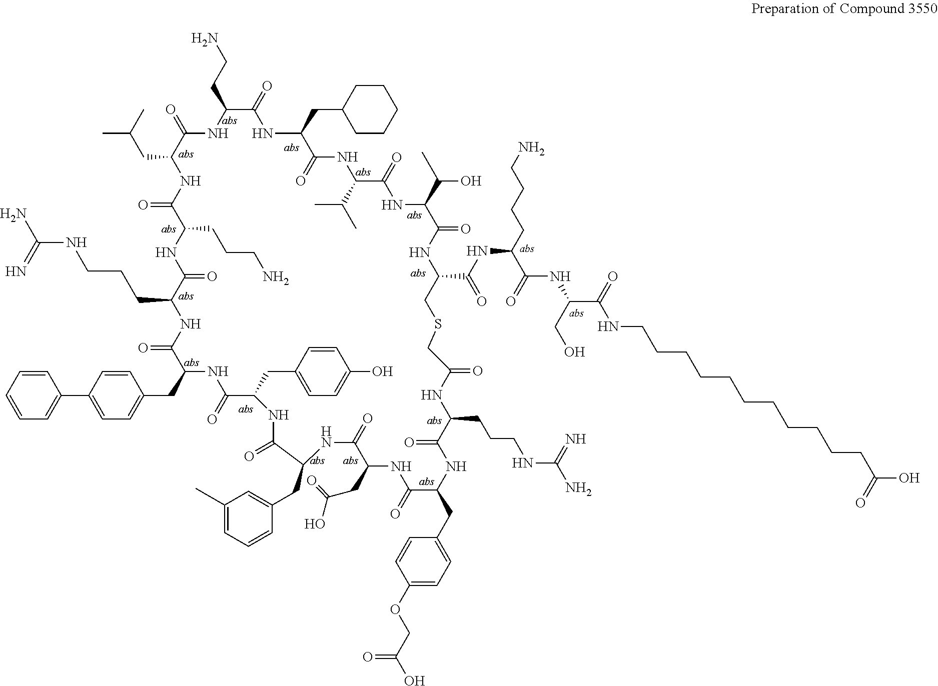 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](Cc3ccc(O)cc3)NC2=O)c1]

Compound 3550 was prepared on a 50 μmol scale. The yield of the product was 9.9 mg, and its estimated purity by LCMS analysis was 96.7%. Analysis condition A: Retention time=1.61 min; ESI-MS(+) m/z [M+2H]²⁺: 1226.2.

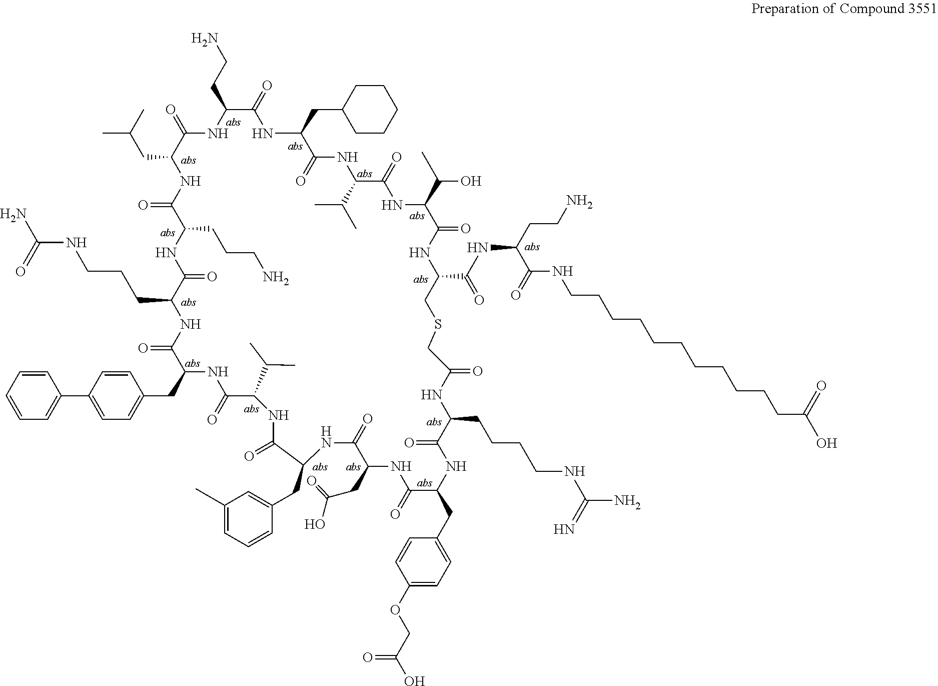 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3551 was prepared on a 50 μmol scale. The yield of the product was 15.9 mg, and its estimated purity by LCMS analysis was 95.5%. Analysis condition B: Retention time=1.61 min; ESI-MS(+) m/z [M+2H]²⁺: 1144.

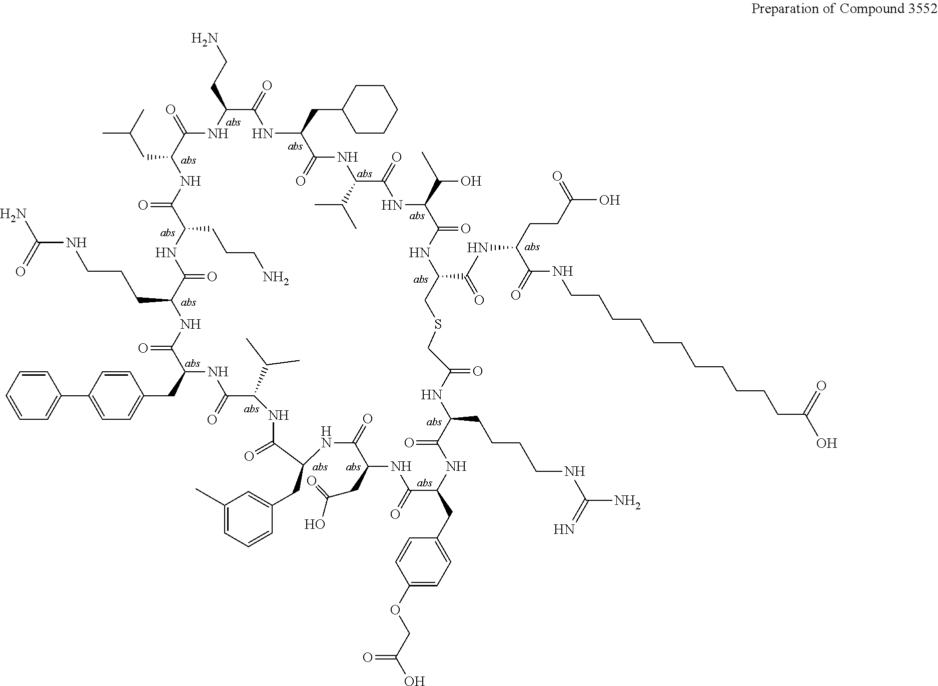 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@H](CCC(=O)O)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3552 was prepared on a 50 μmol scale. The yield of the product was 3.7 mg, and its estimated purity by LCMS analysis was 100%. Analysis condition A: Retention time=1.71 min; ESI-MS(+) m/z [M+3H]³⁺: 773.1.

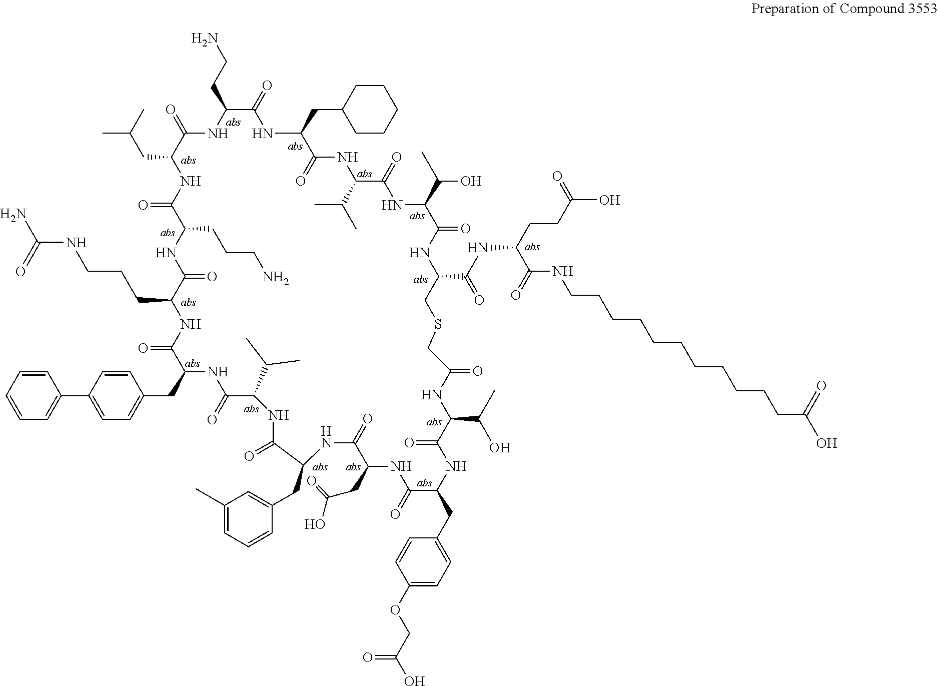 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CCC(=O)O)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3553 was prepared on a 50 μmol scale. The yield of the product was 5.9 mg, and its estimated purity by LCMS analysis was 97.1%. Analysis condition B: Retention time=1.92 min; ESI-MS(+) m/z [M+2H]²⁺: 1124.1.

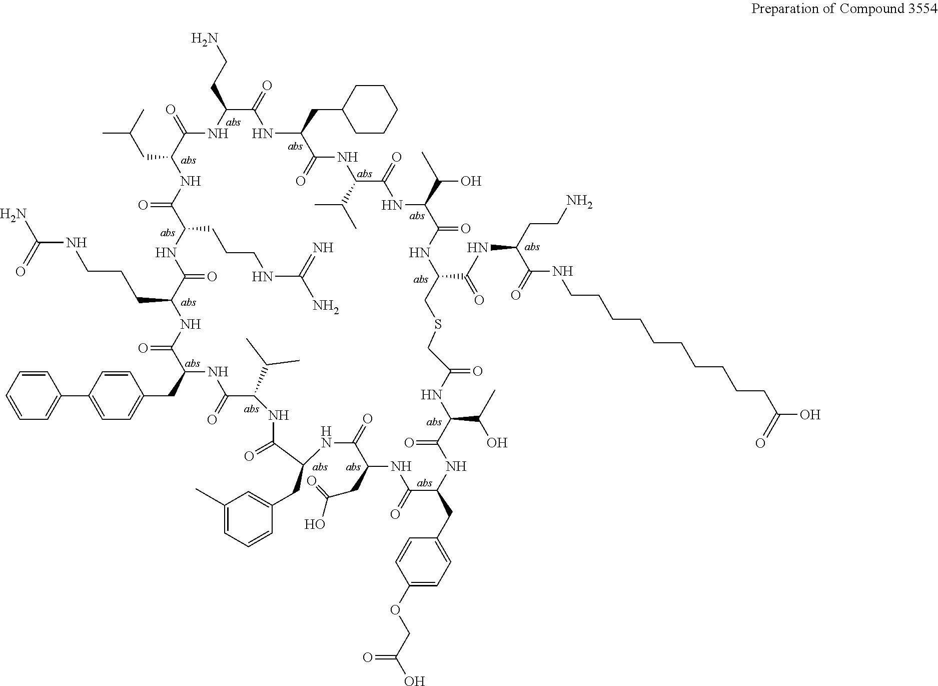 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(=N)N)NC(=O)[C@H](CCCNC(N)=O)NC(=O)[C@H](Cc3ccc(-c4ccccc4)cc3)NC(=O)[C@H](C(C)C)NC2=O)c1]

Compound 3554 was prepared on a 50 μmol scale. The yield of the product was 29.3 mg, and its estimated purity by LCMS analysis was 97.6%. Analysis condition A: Retention time=1.96 min; ESI-MS(+) m/z [M+3H]³⁺: 749.1.

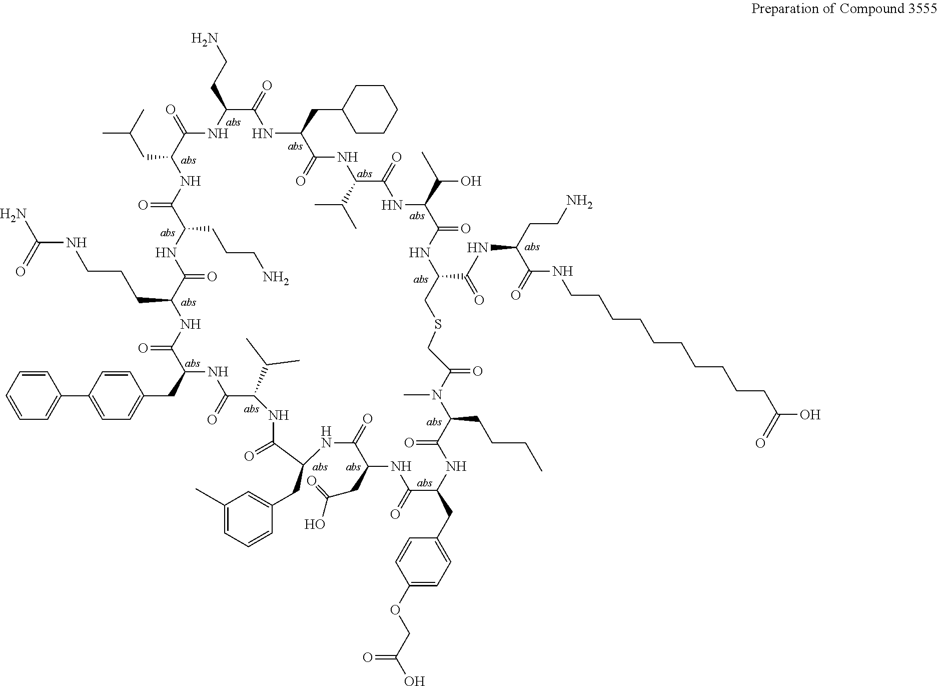 [SMILES: CCCC[C@H]1C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](C(C)O)C(=O)N[C@H](C(=O)N[C@@H](CCN)C(=O)NCCCCCCCCCCC(=O)O)CSCC(=O)N1C]

Compound 3555 was prepared on a 50 μmol scale. The yield of the product was 7.4 mg, and its estimated purity by LCMS analysis was 99.2%. Analysis condition A: Retention time=1.88 min; ESI-MS(+) m/z [M+3H]³⁺: 744.2.

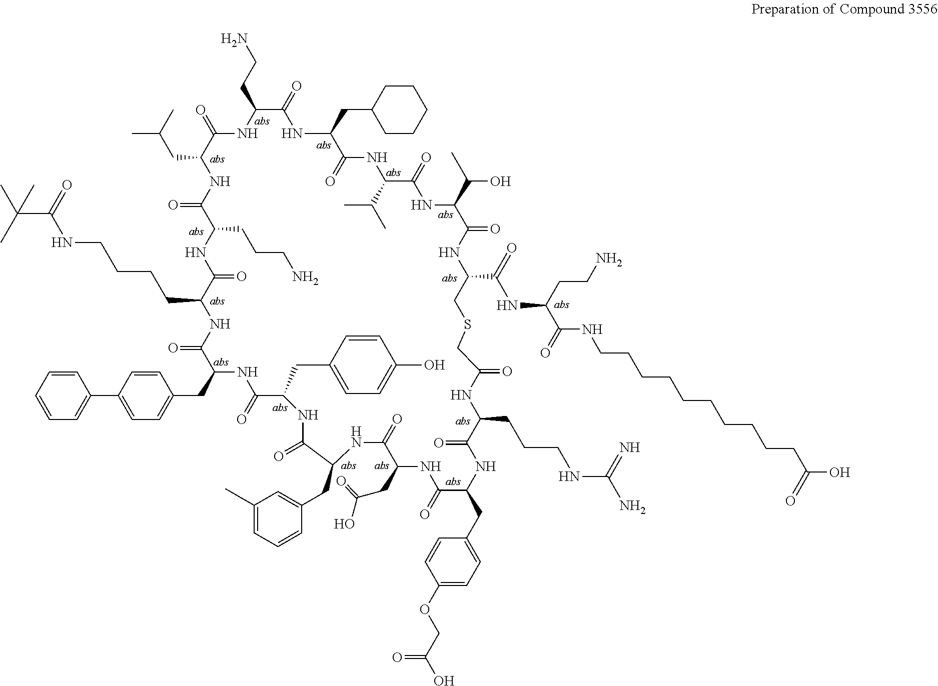 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCCNC(=O)C(C)(C)C)NC(=O)[C@H](Cc3ccc(-c4ccccc4)cc3)NC(=O)[C@H](Cc3ccc(O)cc3)NC2=O)c1]

Compound 3556 was prepared on a 50 μmol scale. The yield of the product was 15.2 mg, and its estimated purity by LCMS analysis was 97%. Analysis condition A: Retention time=1.67 min; ESI-MS(+) m/z [M+2H]²⁺: 1190.1.

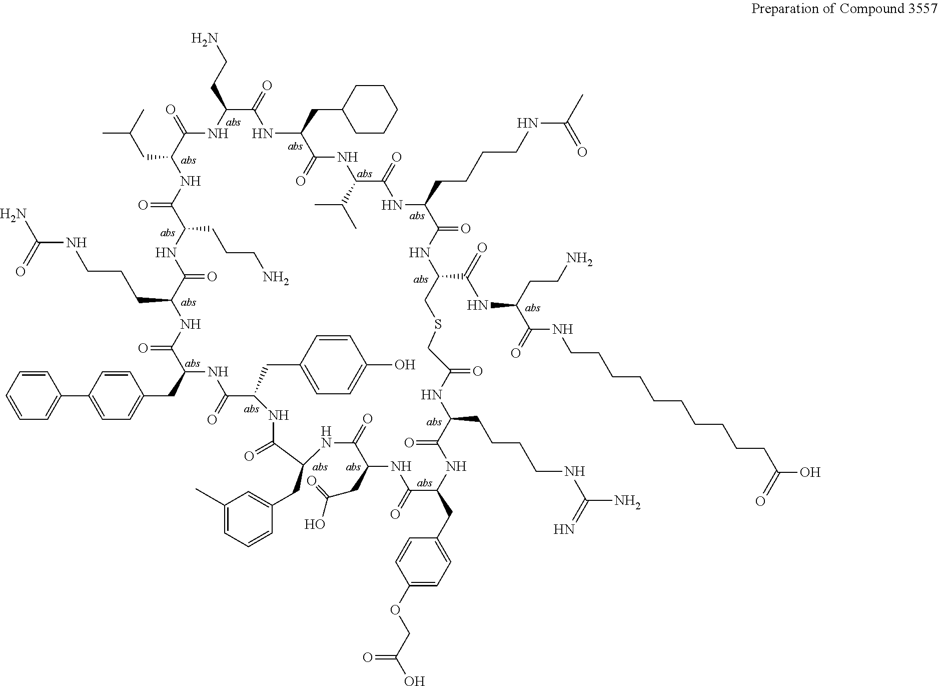 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC1=O]

Compound 3557 was prepared on a 50 μmol scale. The yield of the product was 5.5 mg, and its estimated purity by LCMS analysis was 98.1%. Analysis condition B: Retention time=1.64 min; ESI-MS(+) m/z [M+3H]³⁺: 798.2.

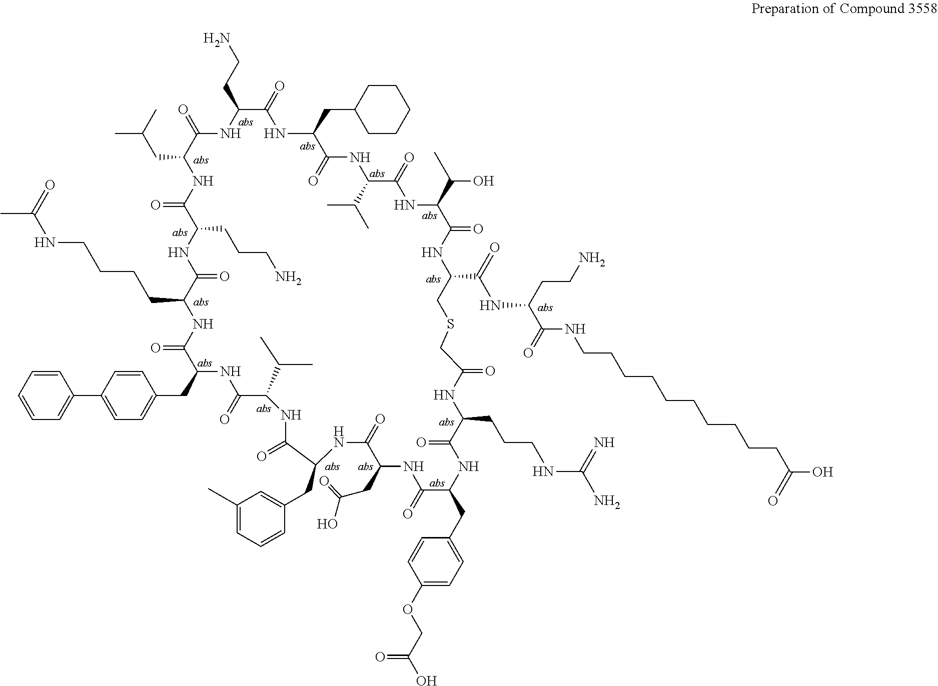 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3558 was prepared on a 50 μmol scale. The yield of the product was 16.9 mg, and its estimated purity by LCMS analysis was 95%. Analysis condition A: Retention time=1.61 min; ESI-MS(+) m/z [M+3H]³⁺: 758.1.

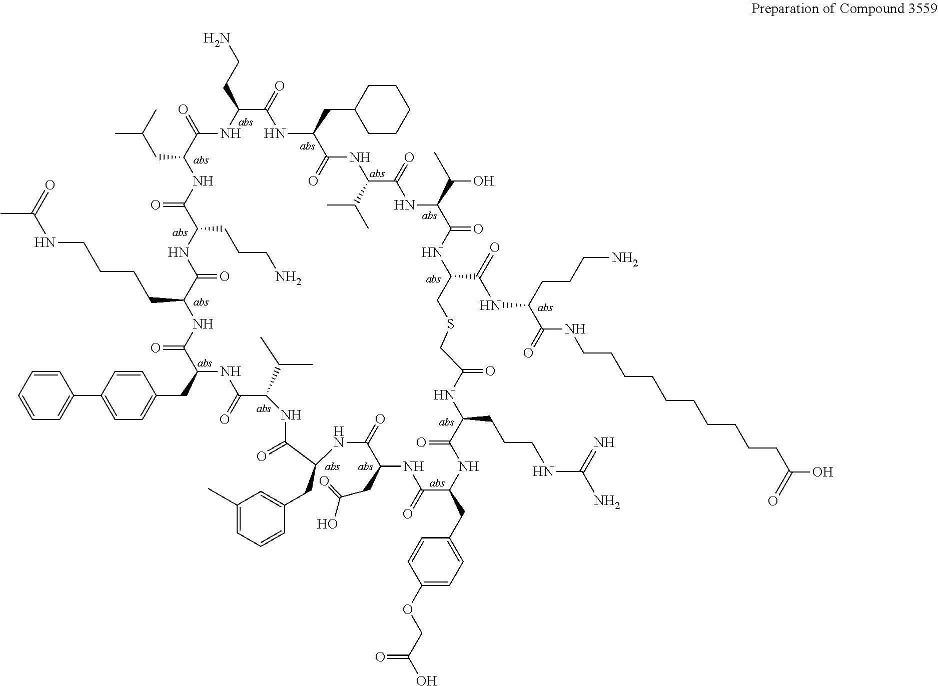 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@H](CCCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3559 was prepared on a 50 μmol scale. The yield of the product was 26.5 mg, and its estimated purity by LCMS analysis was 97.4%. Analysis condition B: Retention time=1.64 min; ESI-MS(+) m/z [M+2H]²⁺: 1143.4.

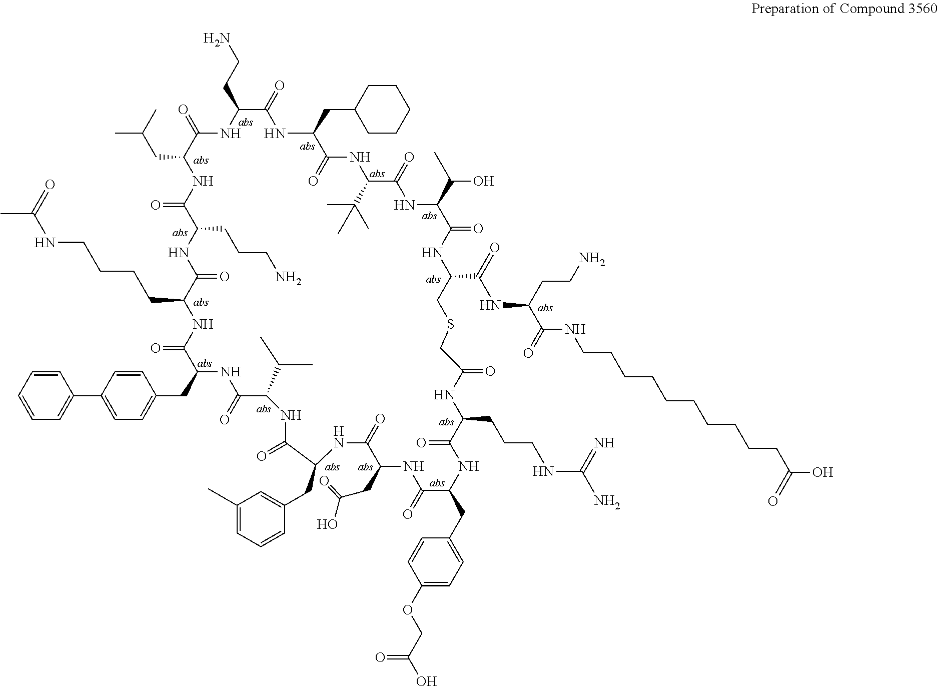 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)(C)C)NC(=O)[C@H](CC2CCCCC2)NC(=O)[C@H](CCN)NC(=O)[C@@H](CC(C)C)NC(=O)[C@H](CCCN)NC1=O]

Compound 3560 was prepared on a 50 μmol scale. The yield of the product was 18 mg, and its estimated purity by LCMS analysis was 100%. Analysis condition A: Retention time=1.56 min; ESI-MS(+) m/z [M+2H]²⁺: 1143.5.

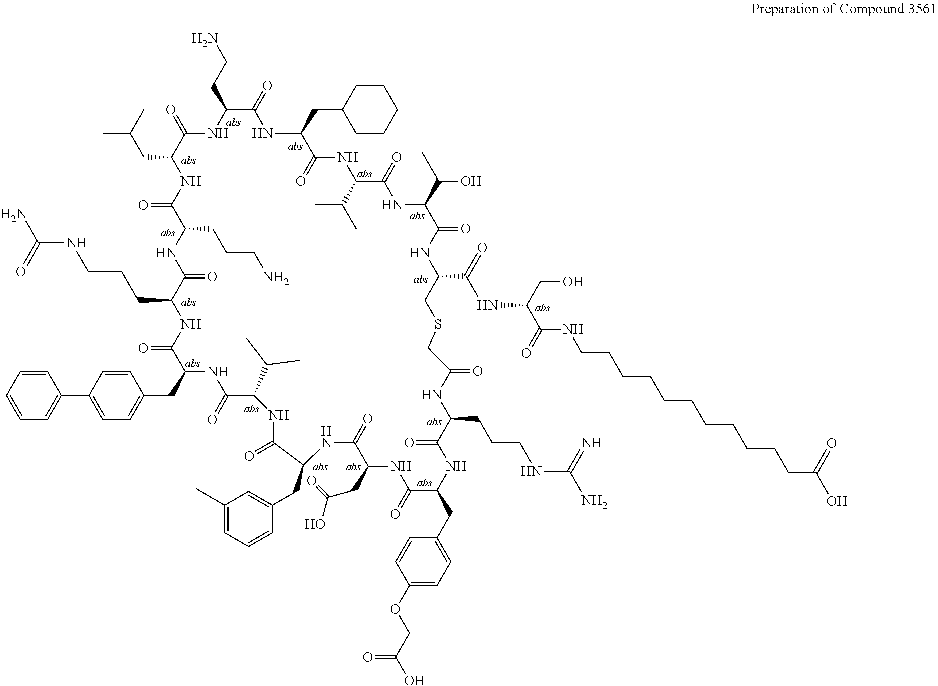 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3561 was prepared on a 50 μmol scale. The yield of the product was 23.8 mg, and its estimated purity by LCMS analysis was 95.2%. Analysis condition A: Retention time=1.7 min; ESI-MS(+) m/z [M+3H]³⁺: 754.

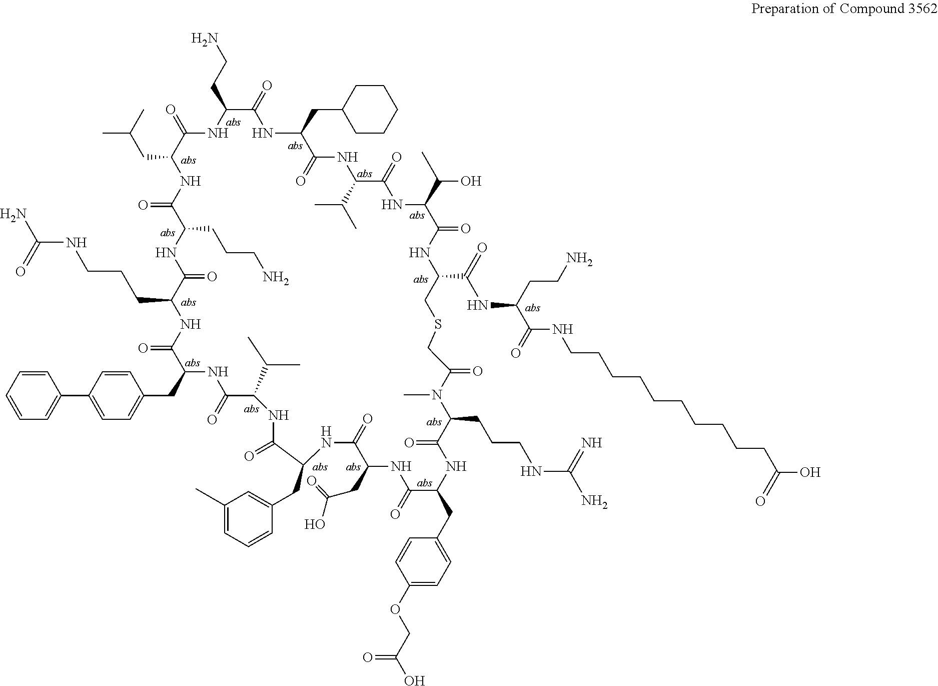 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)N(C)C(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3562 was prepared on a 50 μmol scale. The yield of the product was 10.5 mg, and its estimated purity by LCMS analysis was 96.6%. Analysis condition A: Retention time=1.64 min; ESI-MS(+) m/z [M+2H]²⁺: 1137.2.

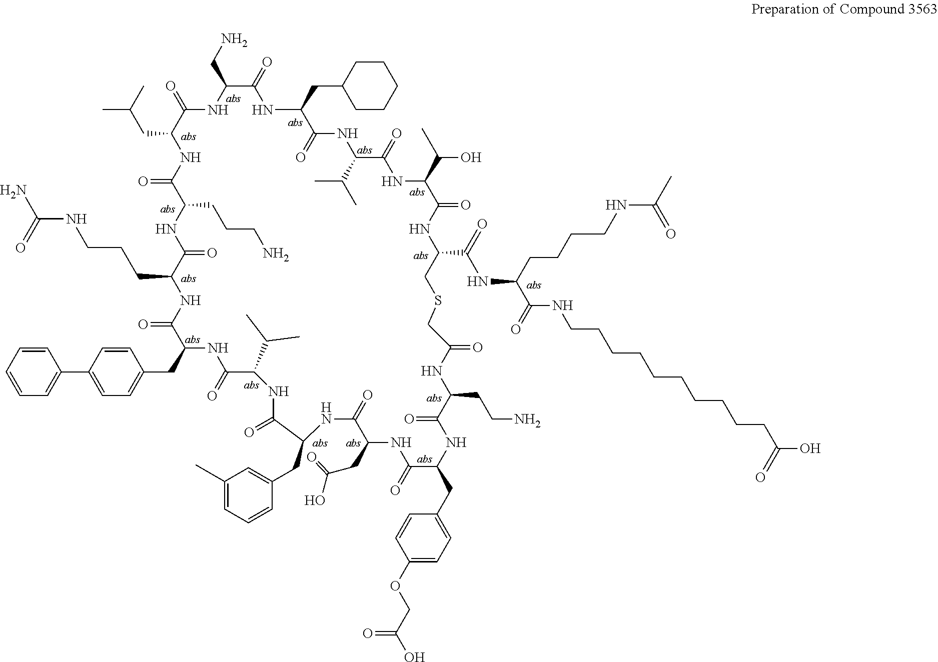 [SMILES: CC(=O)NCCCC[C@H](NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CN)C(=O)N[C@@H](CC2CCCCC2)C(=O)N[C@@H](C(C)C)C(=O)N[C@@H](C(C)O)C(=O)N1)C(=O)NCCCCCCCCCCC(=O)O]

Compound 3563 was prepared on a 50 μmol scale. The yield of the product was 1.8 mg, and its estimated purity by LCMS analysis was 100%. Analysis condition A: Retention time=1.66 min; ESI-MS(+) m/z [M+2H]²⁺: 1130.2.

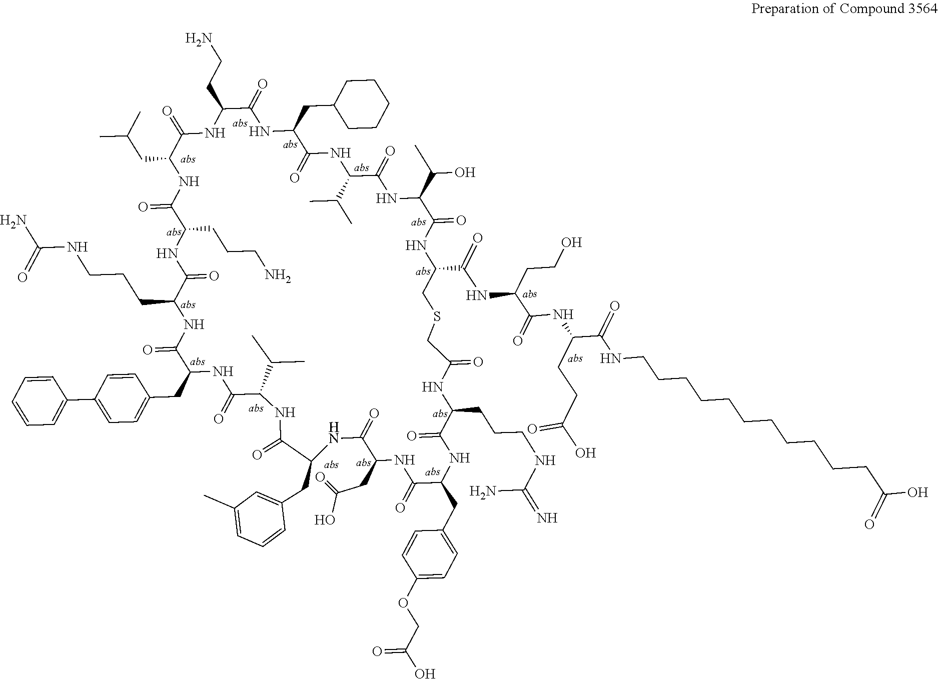 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCO)C(=O)N[C@@H](CCC(=O)O)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3564 was prepared on a 50 μmol scale. The yield of the product was 5.6 mg, and its estimated purity by LCMS analysis was 95.4%. Analysis condition A: Retention time=1.65, 1.7 min; ESI-MS(+) m/z [M+2H]²⁺: 1202.18, 1202.18.

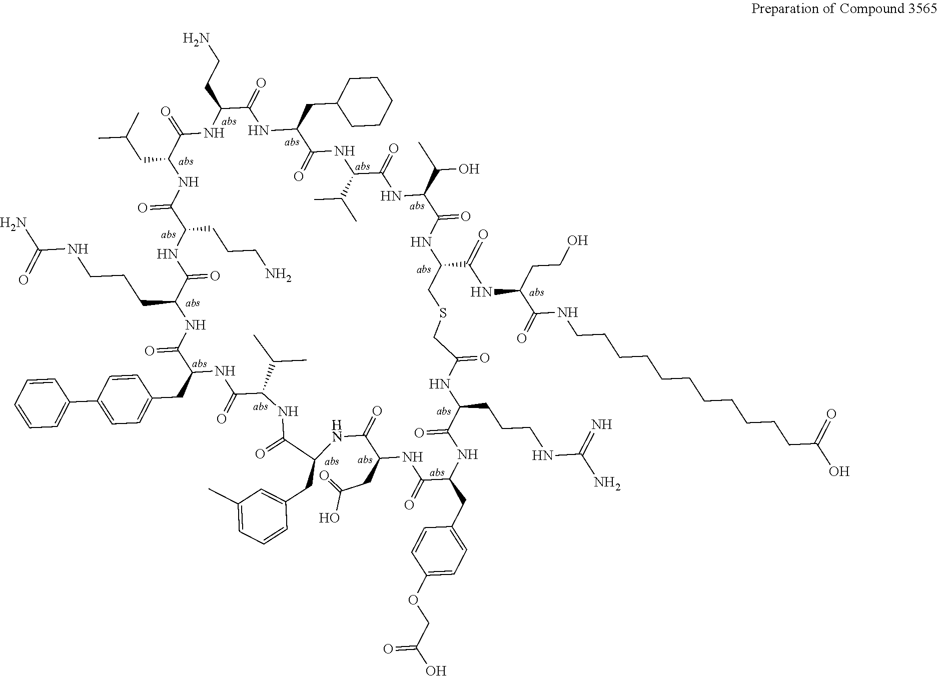 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3565 was prepared on a 50 μmol scale. The yield of the product was 3.3 mg, and its estimated purity by LCMS analysis was 100%. Analysis condition A: Retention time=1.64 min; ESI-MS(+) m/z [M+2H]²⁺: 1137.2.

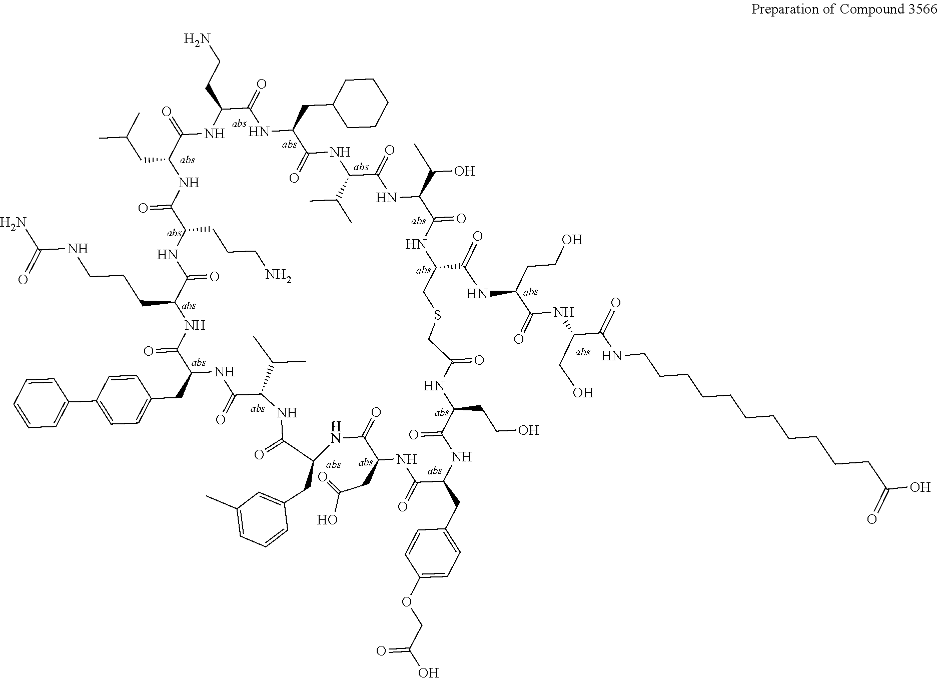 [SMILES: Cc1cccc(C[C@@H]2NC(=O)[C@H](CC(=O)O)NC(=O)[C@H](Cc3ccc(OCC(=O)O)cc3)NC(=O)[C@H](CCO)NC(=O)CSC[C@@H](C(=O)N[C@@H](CCO)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3566 was prepared on a 50 μmol scale. The yield of the product was 2.9 mg, and its estimated purity by LCMS analysis was 100%. Analysis condition A: Retention time=1.61 min; ESI-MS(+) m/z [M+3H]³⁺: 769.3.

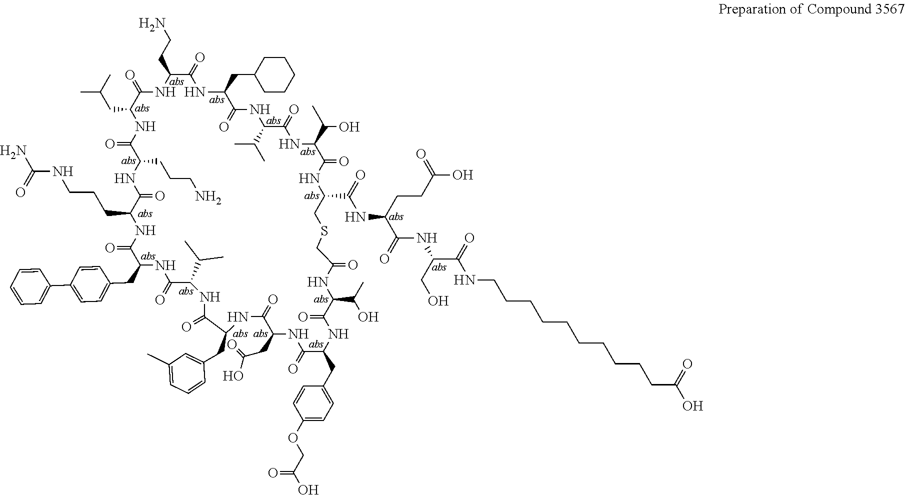 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCC(=O)O)C(=O)N[C@@H](CO)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3567 was prepared on a 50 μmol scale. The yield of the product was 9.8 mg, and its estimated purity by LCMS analysis was 95.8%. Analysis condition A: Retention time=1.54 min; ESI-MS(+) m/z [M+2H]²⁺: 1160.

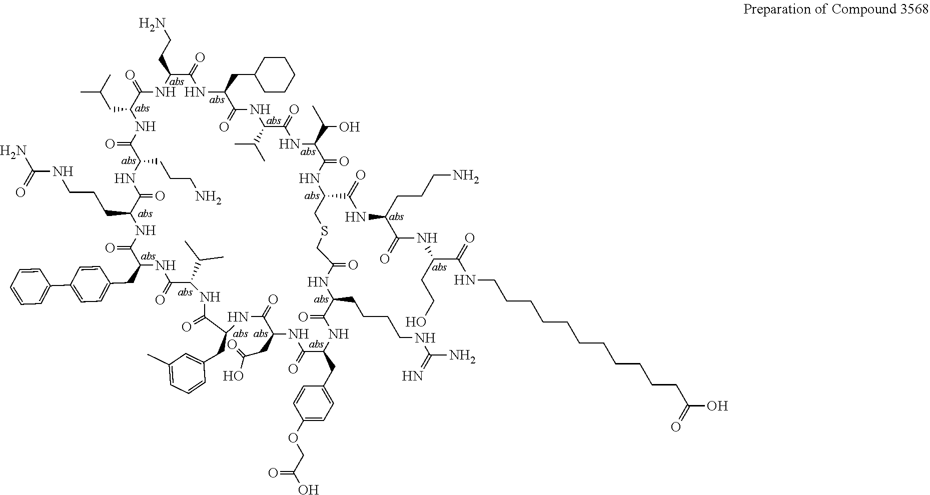 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CCO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3568 was prepared on a 50 μmol scale. The yield of the product was 15.3 mg, and its estimated purity by LCMS analysis was 97.2%. Analysis condition B: Retention time=1.66 min; ESI-MS(+) m/z [M+3H]³⁺: 801.2.

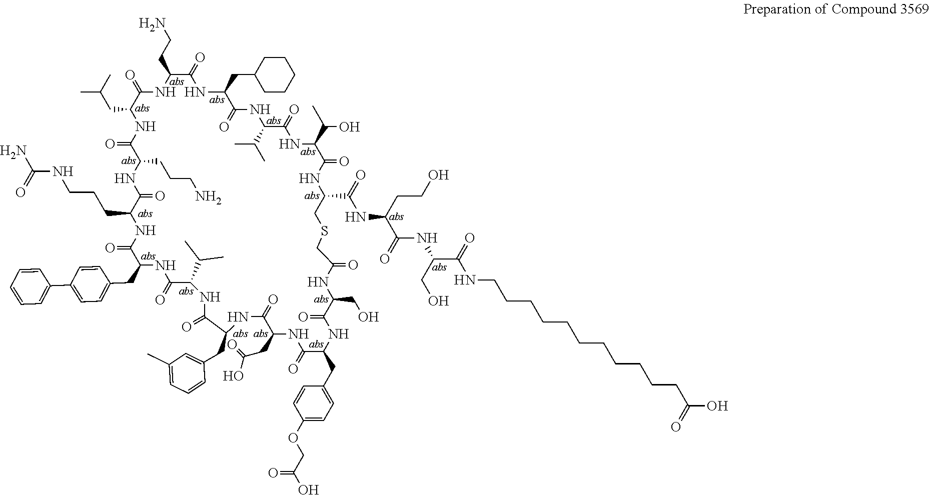 [SMILES: Cc1cccc(C[C@@H]2NC(=O)[C@H](CC(=O)O)NC(=O)[C@H](Cc3ccc(OCC(=O)O)cc3)NC(=O)[C@H](CO)NC(=O)CSC[C@@H](C(=O)N[C@@H](CCO)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3569 was prepared on a 50 μmol scale. The yield of the product was 5.3 mg, and its estimated purity by LCMS analysis was 95.7%. Analysis condition A: Retention time=1.53 min; ESI-MS(+) m/z [M+2H]²⁺: 1146.3.

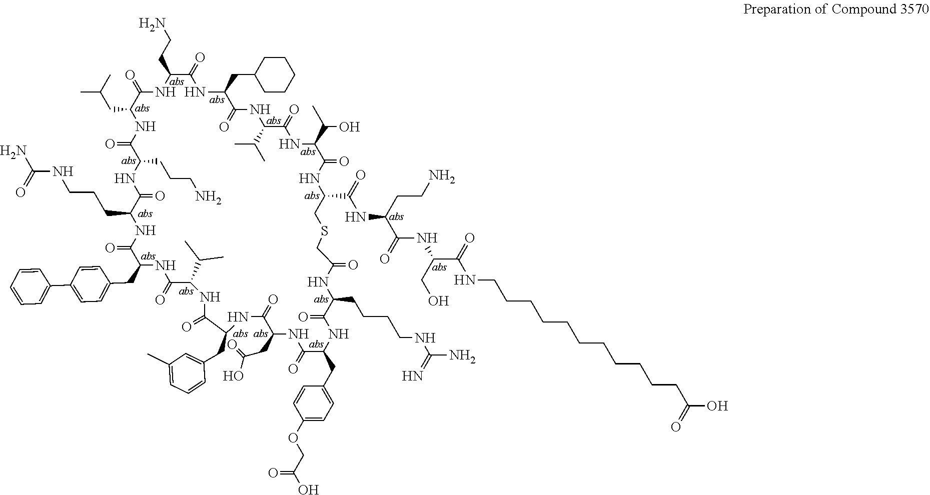 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3570 was prepared on a 50 μmol scale. The yield of the product was 11.4 mg, and its estimated purity by LCMS analysis was 95.7%. Analysis condition B: Retention time=1.51 min; ESI-MS(+) m/z [M+3H]³⁺: 792.

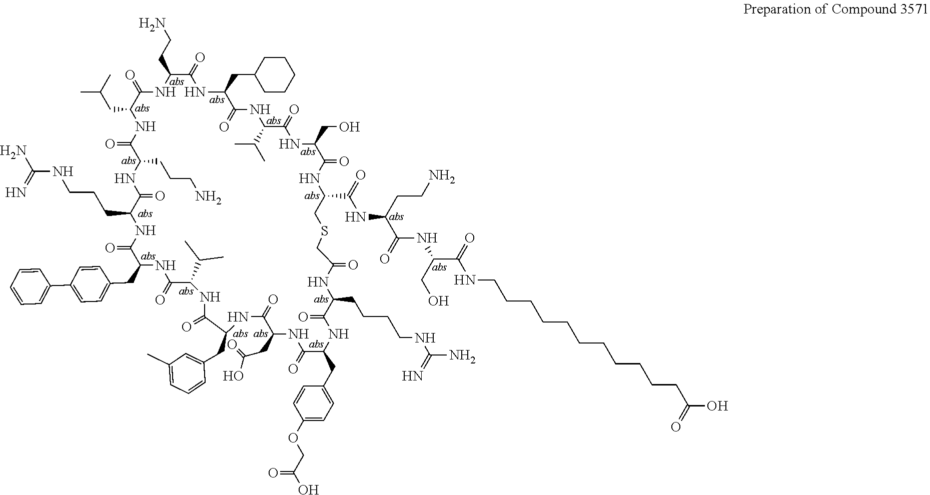 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)N[C@@H](CO)C(=O)NCCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 3571 was prepared on a 50 μmol scale. The yield of the product was 2 mg, and its estimated purity by LCMS analysis was 95.5%. Analysis condition A: Retention time=1.6 min; ESI-MS(+) m/z [M+2H]²⁺: 1180.2.

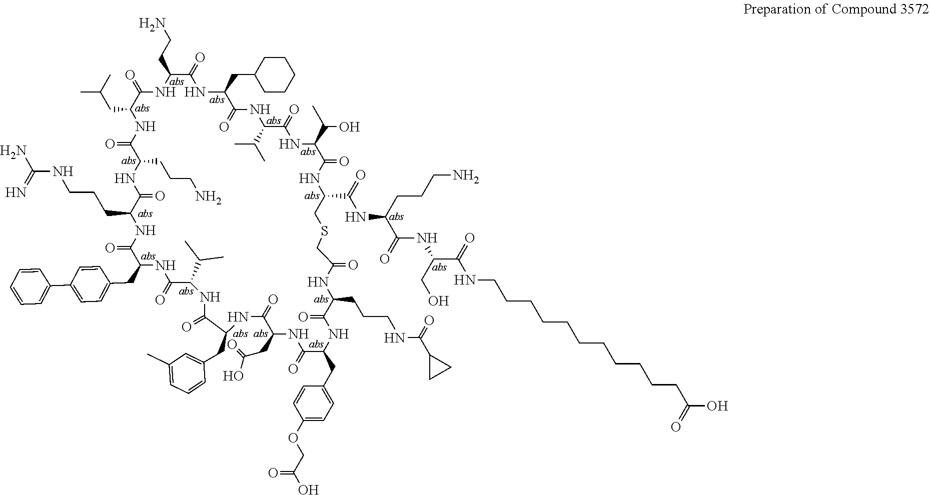 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=O)C3CC3)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 3572 was prepared on a 50 μmol scale. The yield of the product was 7.3 mg, and its estimated purity by LCMS analysis was 100%. Analysis condition A: Retention time=1.73 min; ESI-MS(+) m/z [M+2H]²⁺: 801.

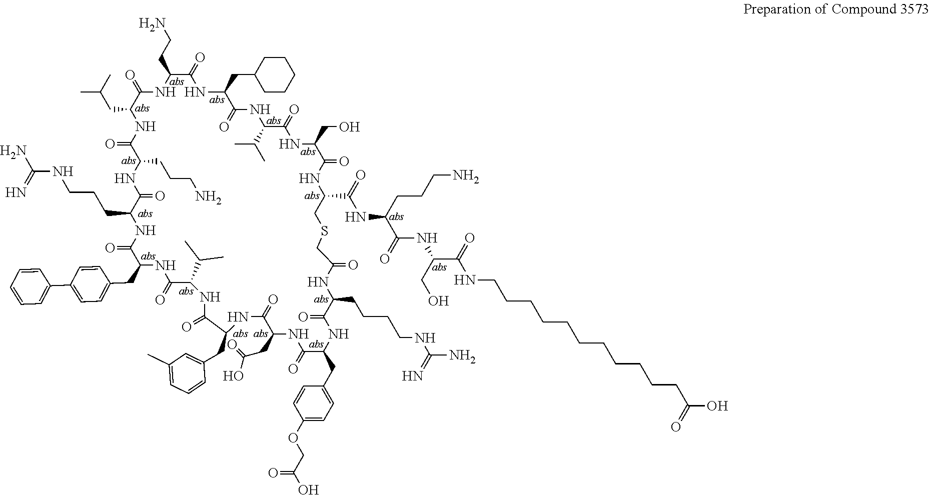 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 3573 was prepared on a 50 μmol scale. The yield of the product was 11.5 mg, and its estimated purity by LCMS analysis was 96%. Analysis condition B: Retention time=1.63 min; ESI-MS(+) m/z [M+2H]²⁺: 1196.3.

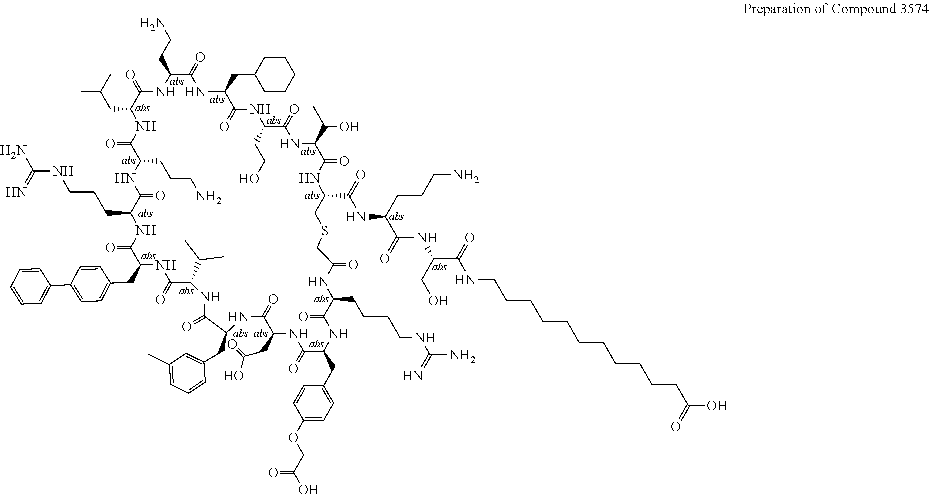 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](CCO)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 3574 was prepared on a 50 μmol scale. The yield of the product was 12.9 mg, and its estimated purity by LCMS analysis was 97.2%. Analysis condition A: Retention time=1.56 min; ESI-MS(+) m/z [M+2H]²⁺: 1195.2.

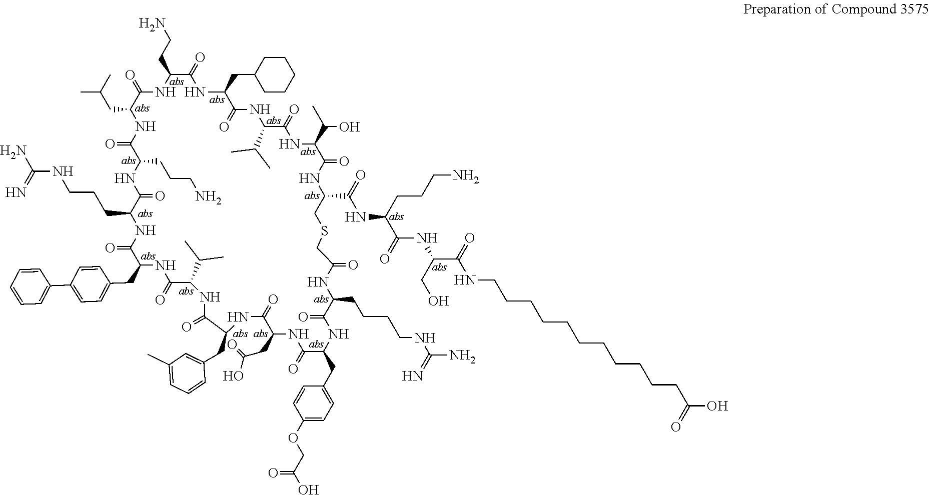 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 3575 was prepared on a 50 μmol scale. The yield of the product was 12.7 mg, and its estimated purity by LCMS analysis was 96.6%. Analysis condition A: Retention time=1.63 min; ESI-MS(+) m/z [M+2H]²⁺: 1194.2.

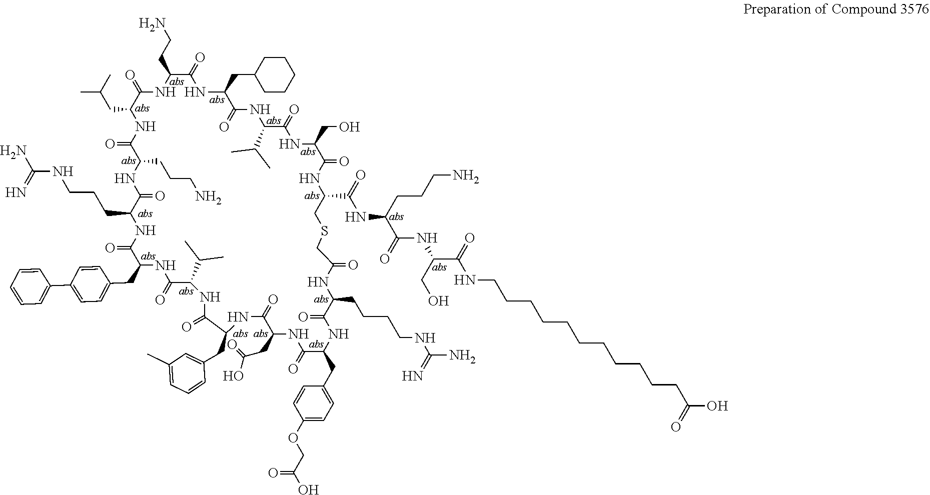 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](C(C)C)NC2=O)c1]

Compound 3576 was prepared on a 50 μmol scale. The yield of the product was 6.8 mg, and its estimated purity by LCMS analysis was 96.2%. Analysis condition B: Retention time=1.59 min; ESI-MS(+) m/z [M+3H]³⁺: 792.2.

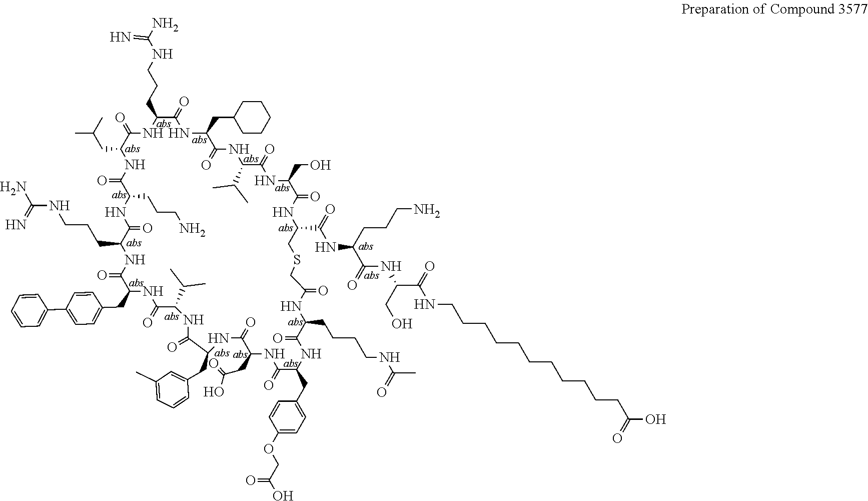 [SMILES: CC(=O)NCCCC[C@@H]1NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](C(C)C)NC(=O)[C@H](CC2CCCCC2)NC(=O)[C@H](CCCNC(=N)N)NC(=O)[C@@H](CC(C)C)NC(=O)[C@H](CCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC1=O]

Compound 3577 was prepared on a 50 μmol scale. The yield of the product was 6.9 mg, and its estimated purity by LCMS analysis was 100%. Analysis condition A: Retention time=1.57, 1.66 min; ESI-MS(+) m/z [M+3H]³⁺: 811.07, 810.9.

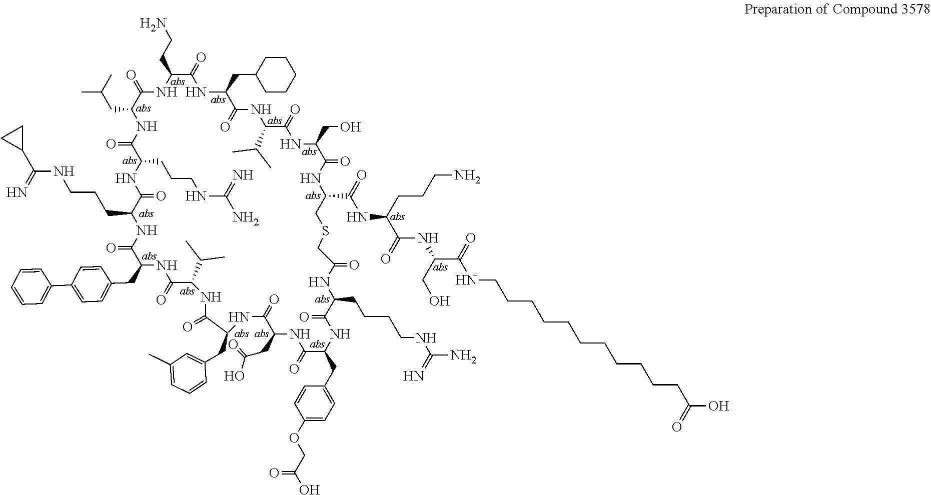 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(=N)N)NC(=O)[C@H](CCCNC(=N)C3CC3)NC(=O)[C@H](Cc3ccc(-c4ccccc4)cc3)NC(=O)[C@H](C(C)C)NC2=O)c1]

Compound 3578 was prepared on a 50 μmol scale. The yield of the product was 3.9 mg, and its estimated purity by LCMS analysis was 86.3%. Analysis condition B: Retention time=1.6 min; ESI-MS(+) m/z [M+2H]²⁺: 1221.1.

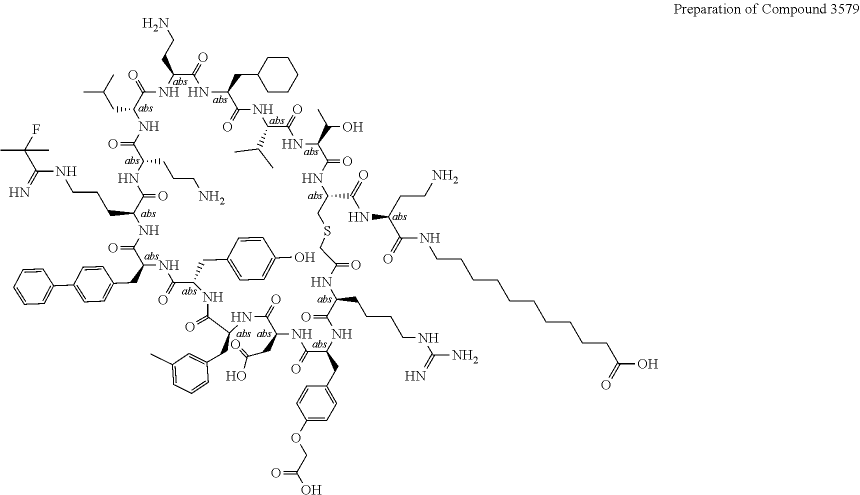 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)C(C)(C)F)NC(=O)[C@H](Cc3ccc(-c4ccccc4)cc3)NC(=O)[C@H](Cc3ccc(O)cc3)NC2=O)c1]

Compound 3579 was prepared on a 50 μmol scale. The yield of the product was 6.4 mg, and its estimated purity by LCMS analysis was 99%. Analysis condition A: Retention time=1.71 min; ESI-MS(+) m/z [M+2H]²⁺: 1192.2.

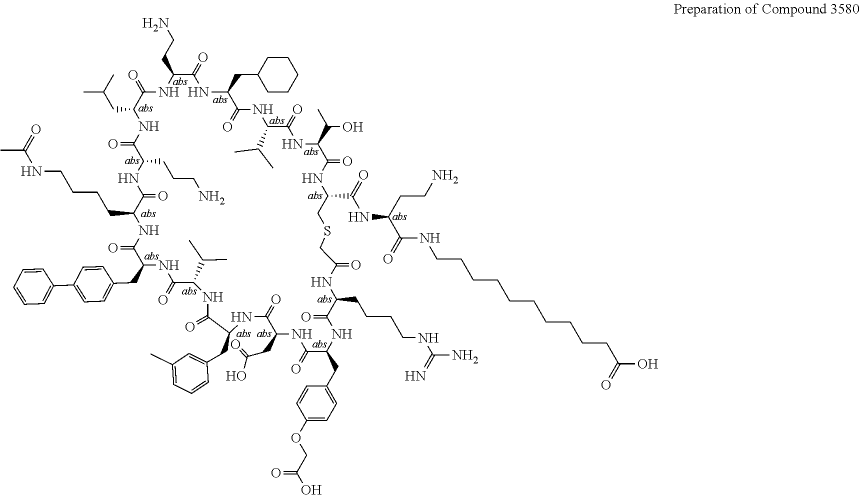 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3580 was prepared on a 50 μmol scale. The yield of the product was 10.8 mg, and its estimated purity by LCMS analysis was 96.9%. Analysis condition A: Retention time=1.62 min; ESI-MS(+) m/z [M+3H]³⁺: 762.1.

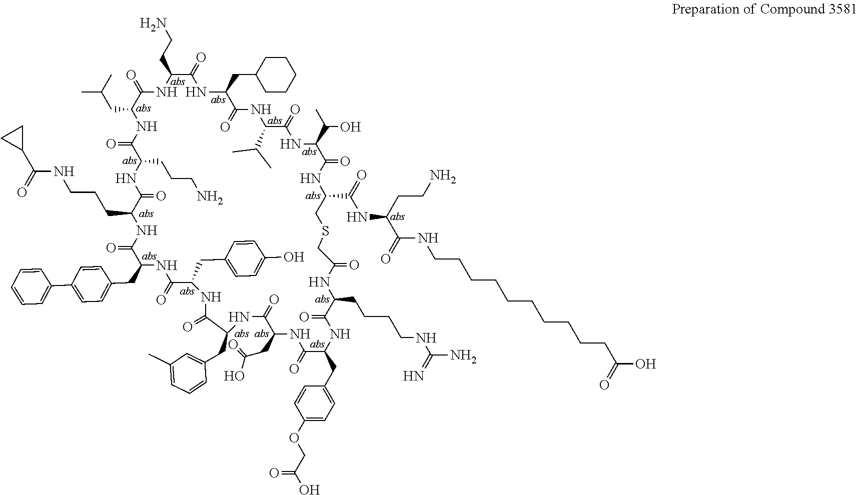 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=O)C3CC3)NC(=O)[C@H](Cc3ccc(-c4ccccc4)cc3)NC(=O)[C@H](Cc3ccc(O)cc3)NC2=O)c1]

Compound 3581 was prepared on a 50 μmol scale. The yield of the product was 3 mg, and its estimated purity by LCMS analysis was 95.6%. Analysis condition A: Retention time=1.68 min; ESI-MS(+) m/z [M+3H]³⁺: 788.2.

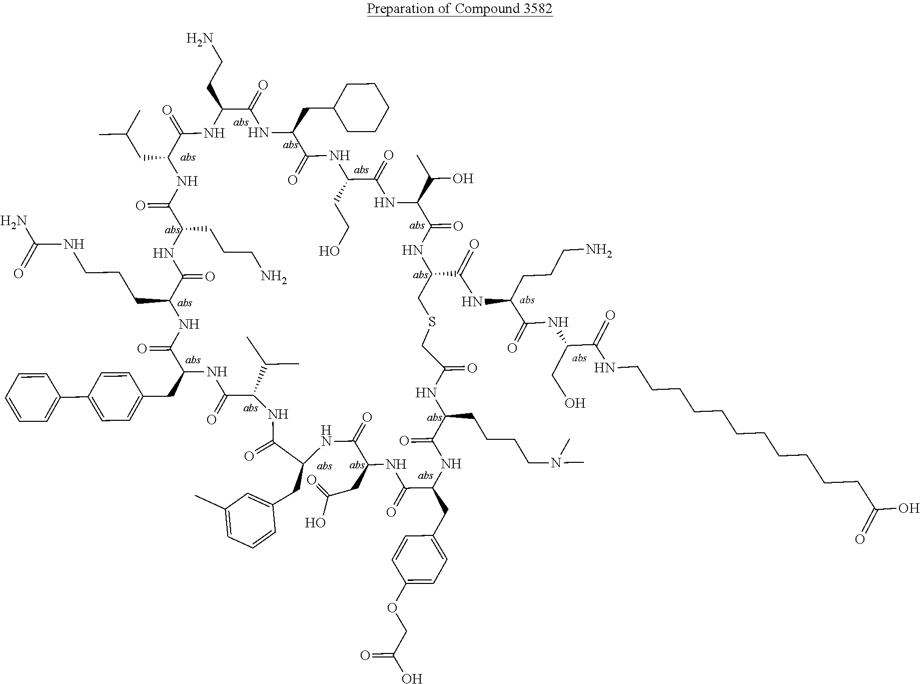 [SMILES: Cc1cccc(C[C@@H]2NC(=O)[C@H](CC(=O)O)NC(=O)[C@H](Cc3ccc(OCC(=O)O)cc3)NC(=O)[C@H](CCCCN(C)C)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](CCO)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3582 was prepared on a 50 μmol scale. The yield of the product was 2.3 mg, and its estimated purity by LCMS analysis was 100%. Analysis condition A: Retention time=1.61 min; ESI-MS(+) m/z [M+3H]³⁺: 793.

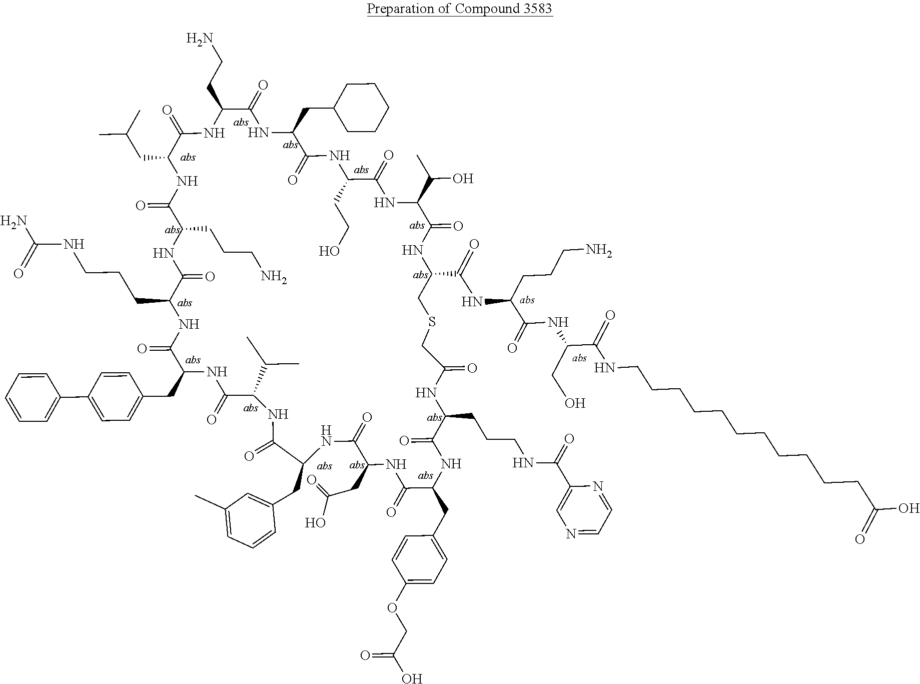 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=O)c3cnccn3)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](CCO)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3583 was prepared on a 50 μmol scale. The yield of the product was 0.9 mg, and its estimated purity by LCMS analysis was 100%. Analysis condition A: Retention time=1.55 min; ESI-MS(+) m/z [M+2H]²⁺: 1221.

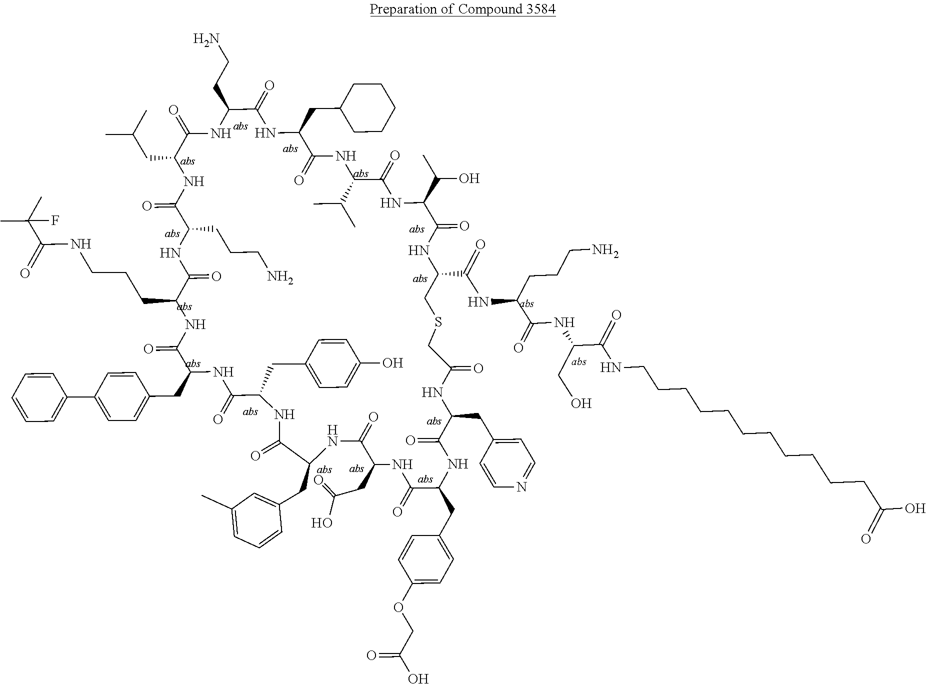 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=O)C(C)(C)F)NC(=O)[C@H](Cc3ccc(-c4ccccc4)cc3)NC(=O)[C@H](Cc3ccc(O)cc3)NC2=O)c1]

Compound 3584 was prepared on a 50 μmol scale. The yield of the product was 10.3 mg, and its estimated purity by LCMS analysis was 87%. Analysis condition B: Retention time=1.65 min; ESI-MS(+) m/z [M+2H]²⁺: 1238.2.

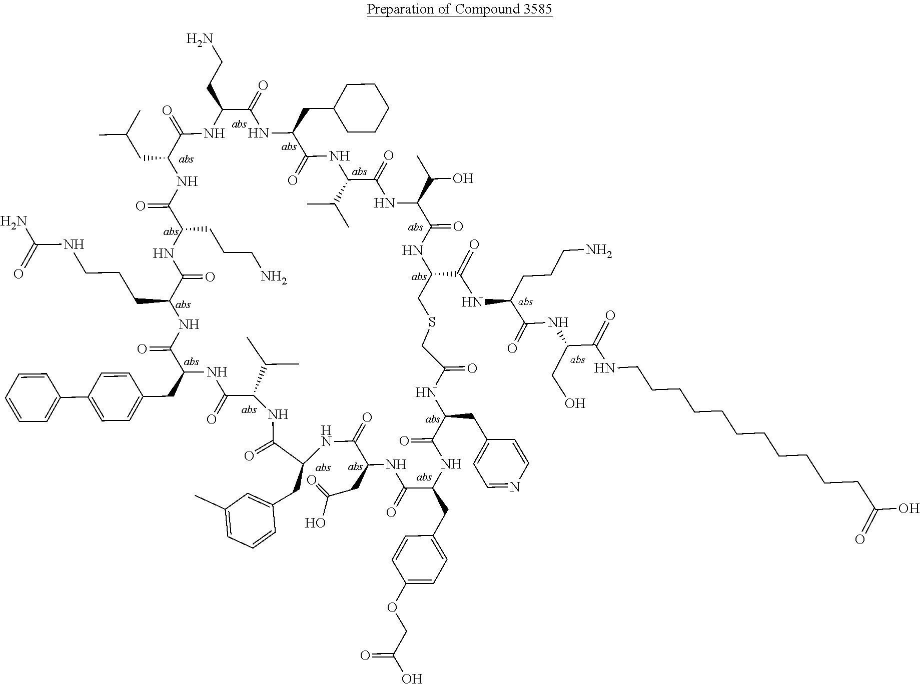 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3585 was prepared on a 50 μmol scale. The yield of the product was 19.1 mg, and its estimated purity by LCMS analysis was 95%. Analysis condition B: Retention time=1.64 min; ESI-MS(+) m/z [M+3H]³⁺: 789.

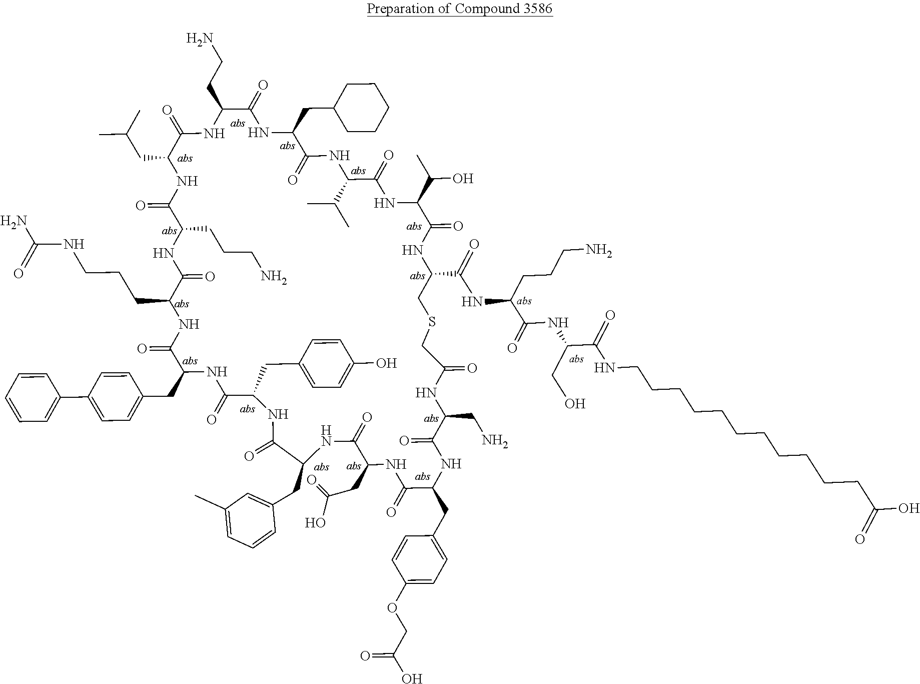 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](Cc3ccc(O)cc3)NC2=O)c1]

Compound 3586 was prepared on a 50 μmol scale. The yield of the product was 17.6 mg, and its estimated purity by LCMS analysis was 88.9%. Analysis condition B: Retention time=1.63 min; ESI-MS(+) m/z [M+3H]³⁺: 790.2.

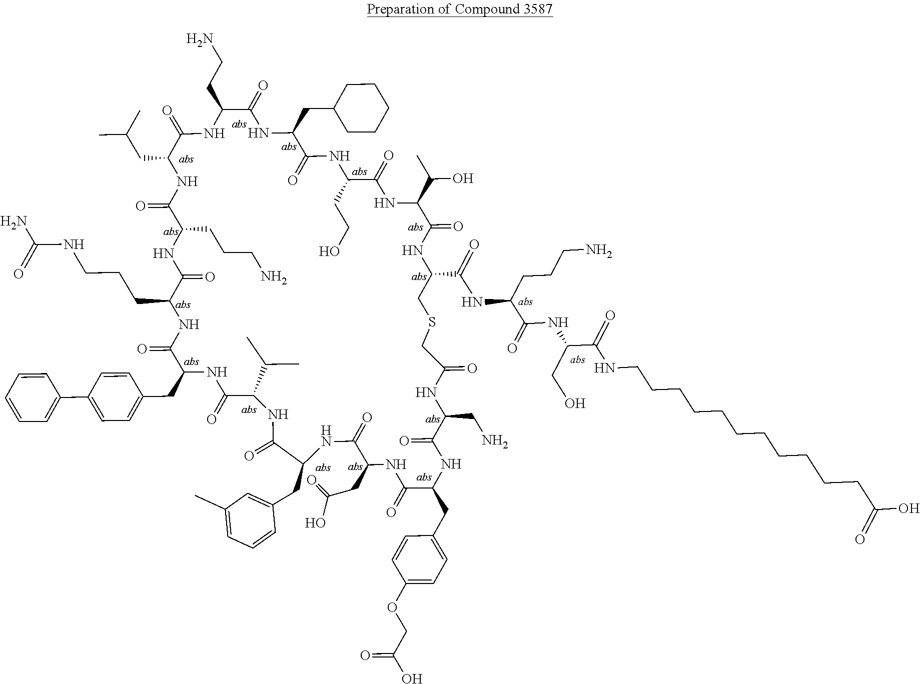 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)N[C@@H](CO)C(=O)NCCCCCCCCCCCC(=O)O)NC(=O)[C@H](C(C)O)NC(=O)[C@H](CCO)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3587 was prepared on a 50 μmol scale. The yield of the product was 9.7 mg, and its estimated purity by LCMS analysis was 97.1%. Analysis condition B: Retention time=1.64 min; ESI-MS(+) m/z [M+3H]³⁺: 769.3.

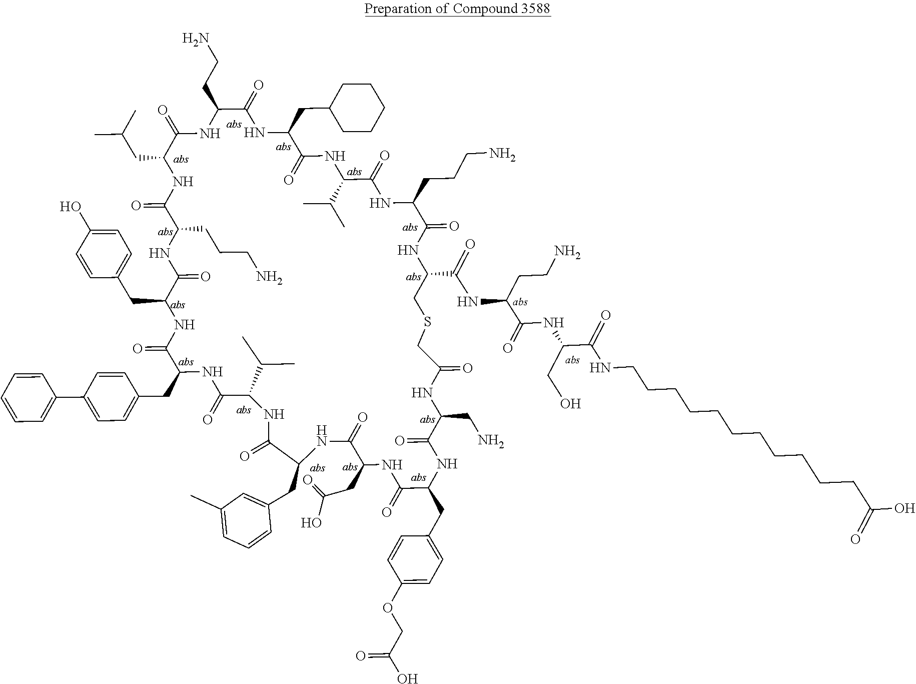 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)N[C@@H](CO)C(=O)NCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3588 was prepared on a 50 μmol scale. The yield of the product was 3.2 mg, and its estimated purity by LCMS analysis was 96.4%. Analysis condition B: Retention time=1.59 min; ESI-MS(+) m/z [M+2H]²⁺: 770.2.

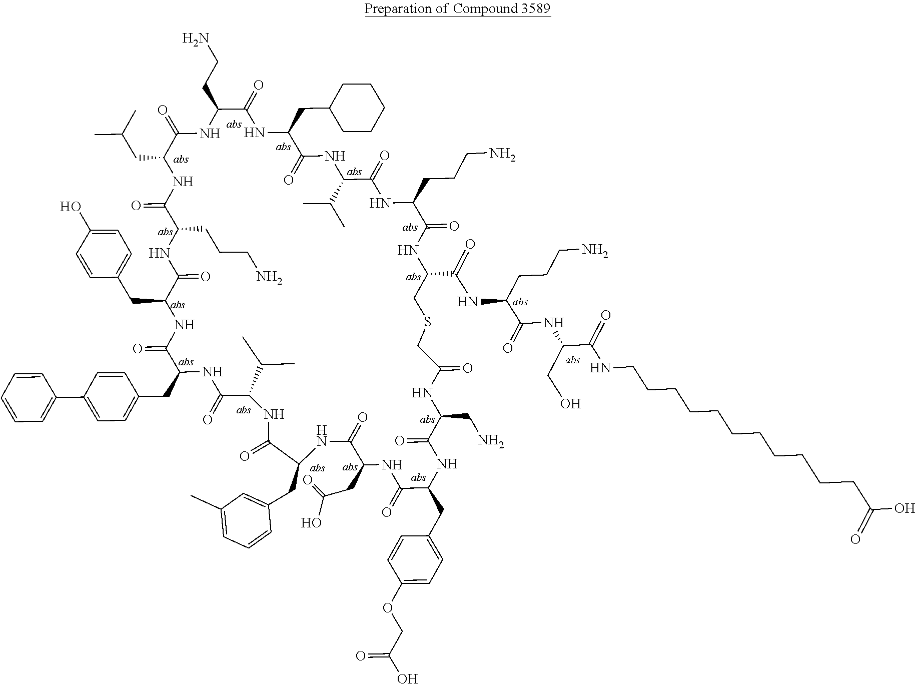 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(=O)N[C@@H](CO)C(=O)NCCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3589 was prepared on a 50 μmol scale. The yield of the product was 1.1 mg, and its estimated purity by LCMS analysis was 96.5%. Analysis condition B: Retention time=1.57 min; ESI-MS(+) m/z [M+2H]²⁺: 1161.3.

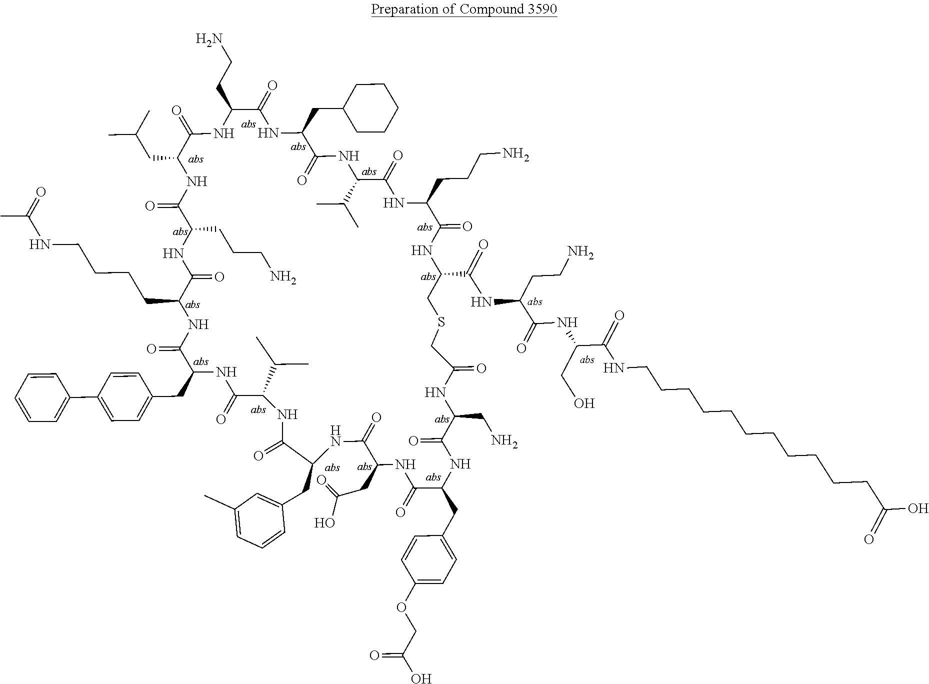 [SMILES: CC(=O)NCCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)N[C@@H](CO)C(=O)NCCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3590 was prepared on a 50 μmol scale. The yield of the product was 23.5 mg, and its estimated purity by LCMS analysis was 96%. Analysis condition A: Retention time=1.68 min; ESI-MS(+) m/z [M+2H]²⁺: 1158.

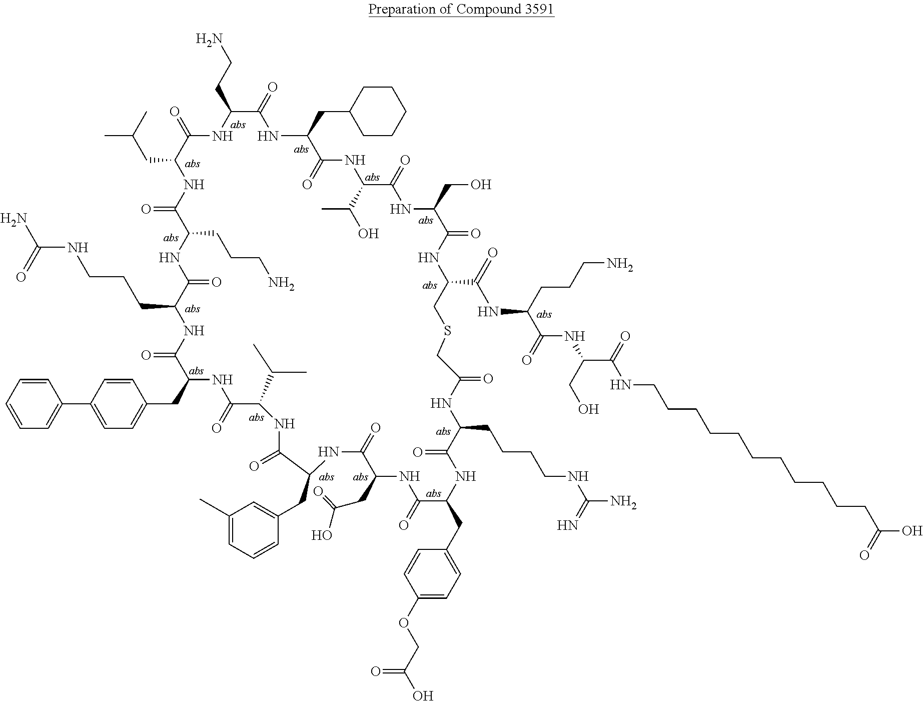 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 3591 was prepared on a 50 μmol scale. The yield of the product was 4.3 mg, and its estimated purity by LCMS analysis was 95.3%. Analysis condition A: Retention time=1.57 min; ESI-MS(+) m/z [M+3H]³⁺: 793.1.

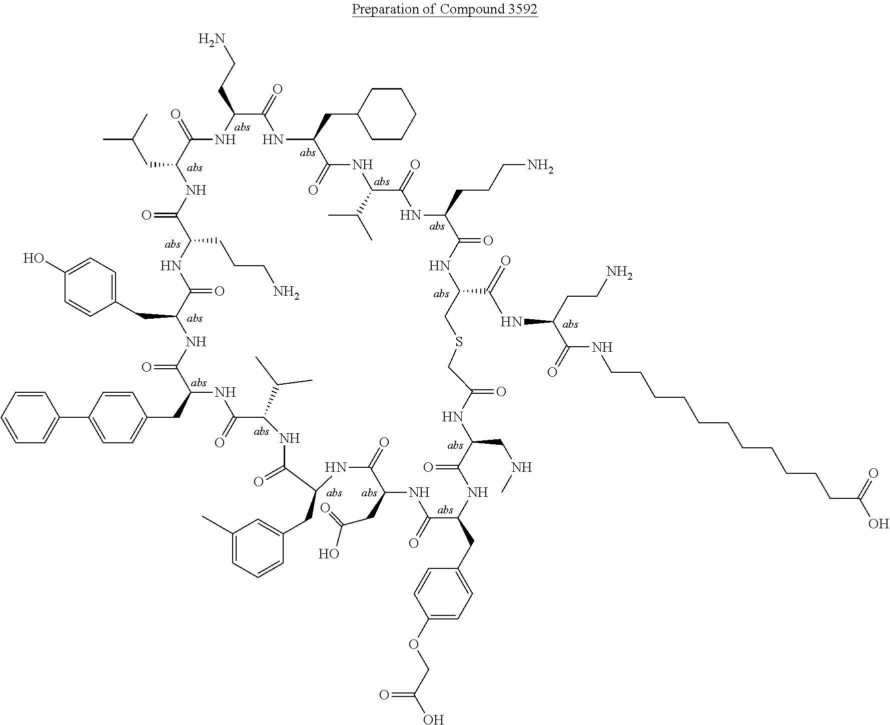 [SMILES: CNC[C@@H]1NC(=O)CSC[C@@H](C(=O)N[C@@H](CCN)C(=O)NCCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC1=O]

Compound 3592 was prepared on a 50 μmol scale. The yield of the product was 26.8 mg, and its estimated purity by LCMS analysis was 96.3%. Analysis condition A: Retention time=1.6 min; ESI-MS(+) m/z [M+3H]³⁺: 746.1.

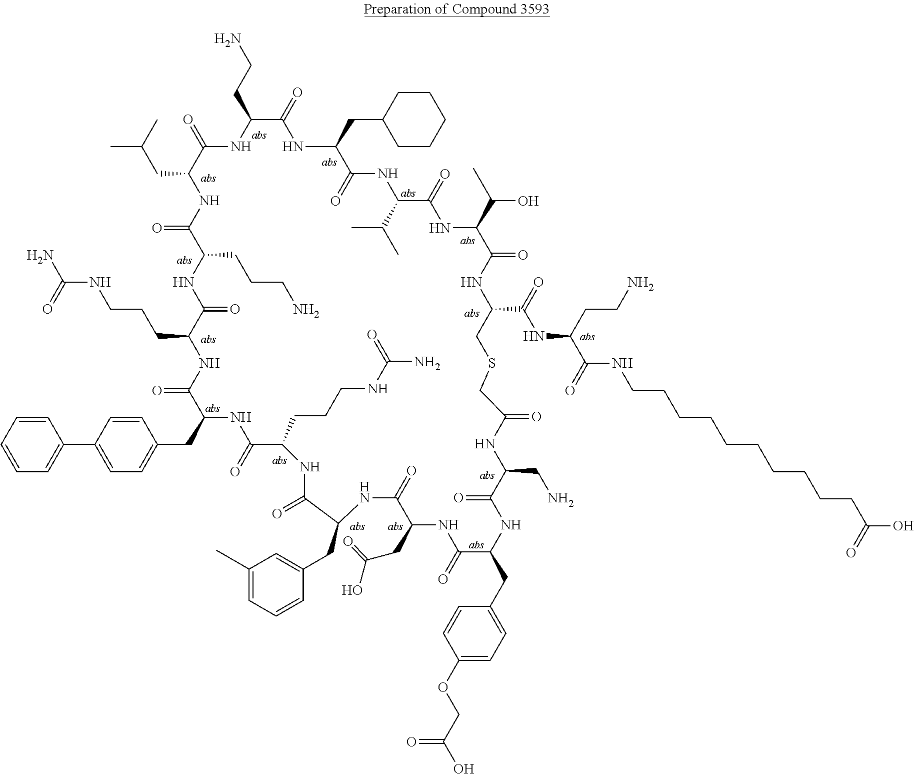 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](CCCNC(N)=O)NC2=O)c1]

Compound 3593 was prepared on a 50 μmol scale. The yield of the product was 1.8 mg, and its estimated purity by LCMS analysis was 96.1%. Analysis condition B: Retention time=1.54 min; ESI-MS(+) m/z [M+2H]²⁺: 1123.5.

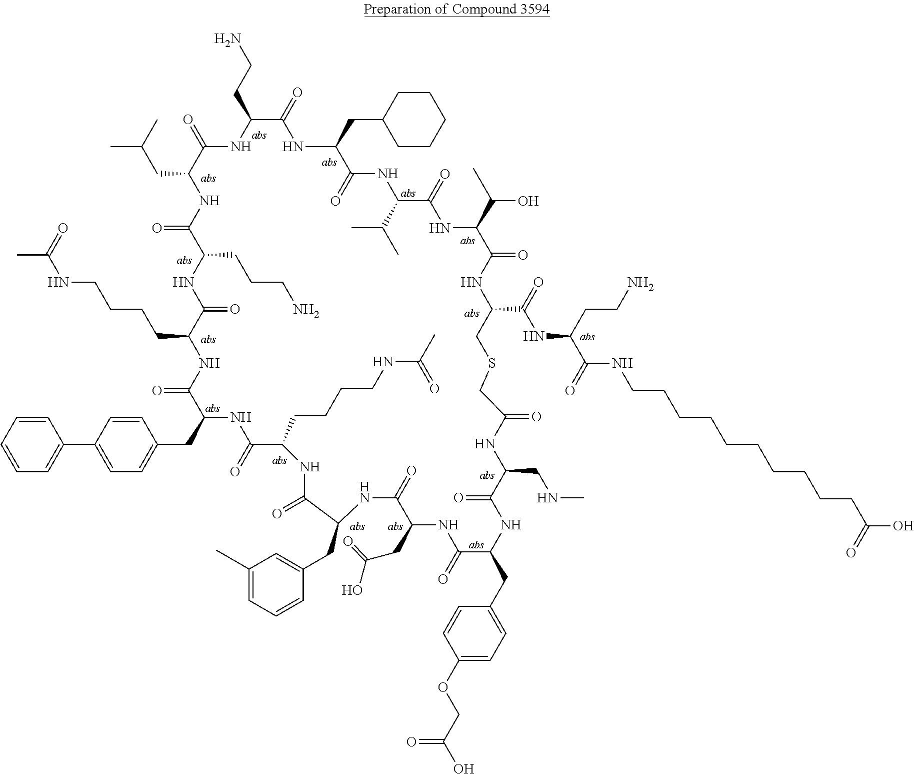 [SMILES: CNC[C@@H]1NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCCNC(C)=O)NC(=O)[C@H](Cc2ccc(-c3ccccc3)cc2)NC(=O)[C@H](CCCCNC(C)=O)NC(=O)[C@H](Cc2cccc(C)c2)NC(=O)[C@H](CC(=O)O)NC(=O)[C@H](Cc2ccc(OCC(=O)O)cc2)NC1=O]

Compound 3594 was prepared on a 50 μmol scale. The yield of the product was 2.1 mg, and its estimated purity by LCMS analysis was 98.1%. Analysis condition B: Retention time=1.56 min; ESI-MS(+) m/z [M+2H]²⁺: 1143.3.

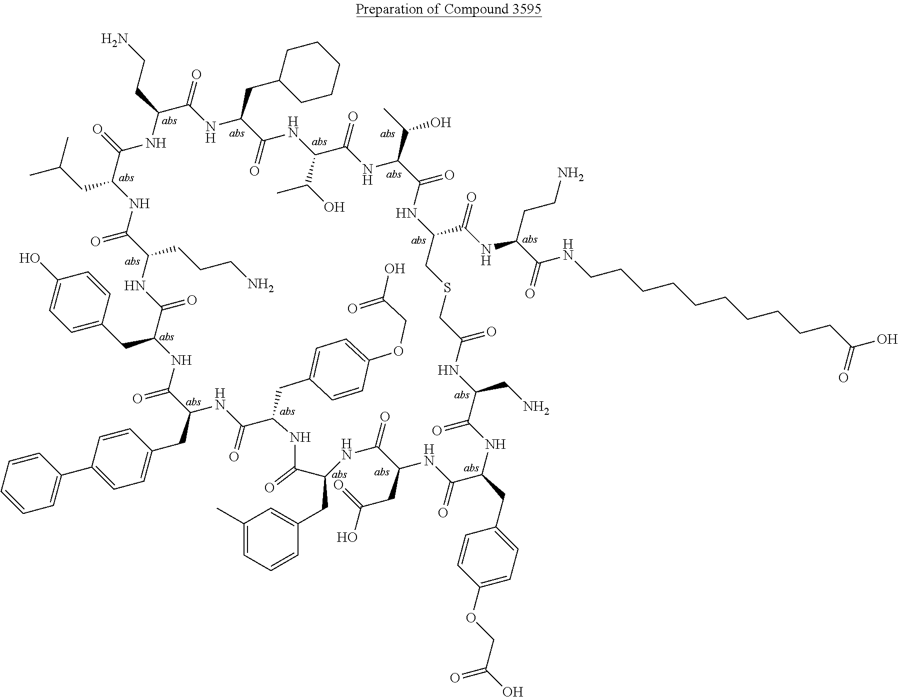 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H]([C@H](C)O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Compound 3595 was prepared on a 50 μmol scale. The yield of the product was 2.8 mg, and its estimated purity by LCMS analysis was 96.5%. Analysis condition A: Retention time=1.57 min; ESI-MS(+) m/z [M+2H]²⁺: 1159.2.

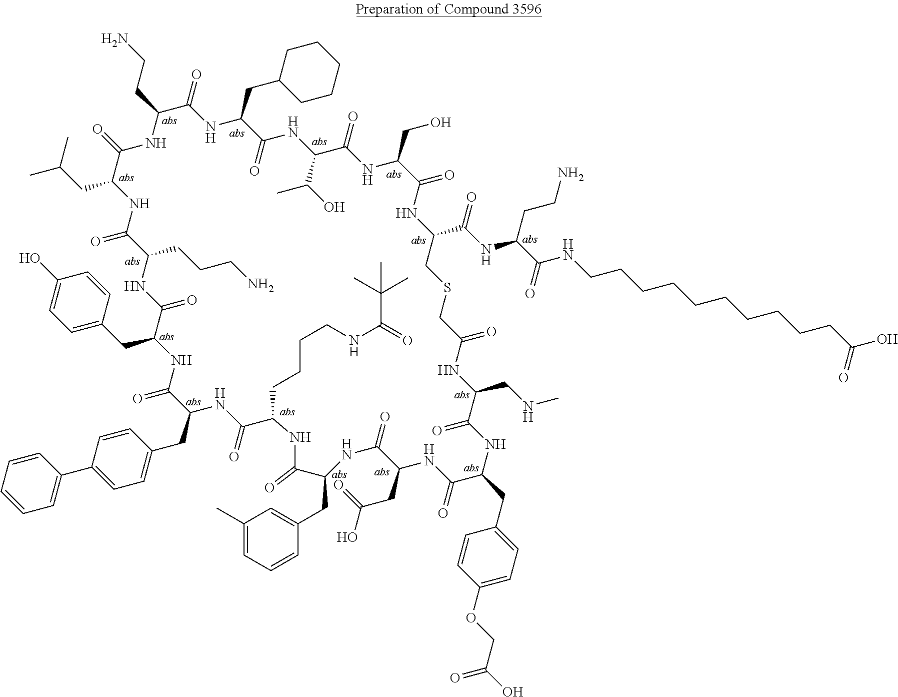 [SMILES: CNC[C@@H]1NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC(=O)[C@H](CCCCNC(=O)C(C)(C)C)NC(=O)[C@H](Cc2cccc(C)c2)NC(=O)[C@H](CC(=O)O)NC(=O)[C@H](Cc2ccc(OCC(=O)O)cc2)NC1=O]

Compound 3596 was prepared on a 50 μmol scale. The yield of the product was 1 mg, and its estimated purity by LCMS analysis was 94.7%. Analysis condition B: Retention time=1.63 min; ESI-MS(+) m/z [M+3H]³⁺: 770.7.

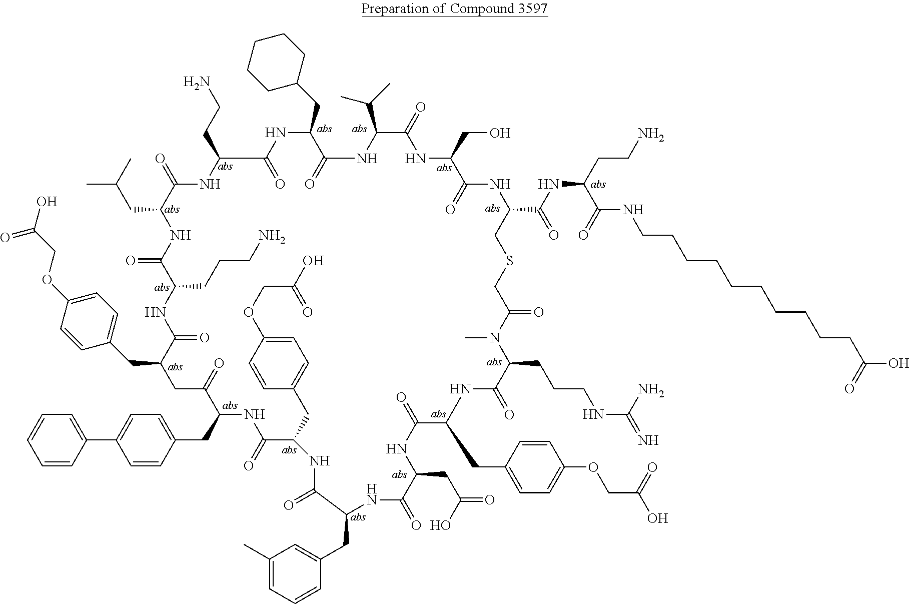 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)N(C)C(=O)CSC[C@@H](C(=O)N[C@@H](CCN)C(=O)NCCCCCCCCCCC(=O)O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)CC(=O)[C@H](Cc3ccc(-c4ccccc4)cc3)NC(=O)[C@H](Cc3ccc(OCC(=O)O)cc3)NC2=O)c1]

Compound 3597 was prepared on a 50 μmol scale. The yield of the product was 2.6 mg, and its estimated purity by LCMS analysis was 97%. Analysis condition B: Retention time=1.57 min; ESI-MS(+) m/z [M+3H]³⁺: 815.5.

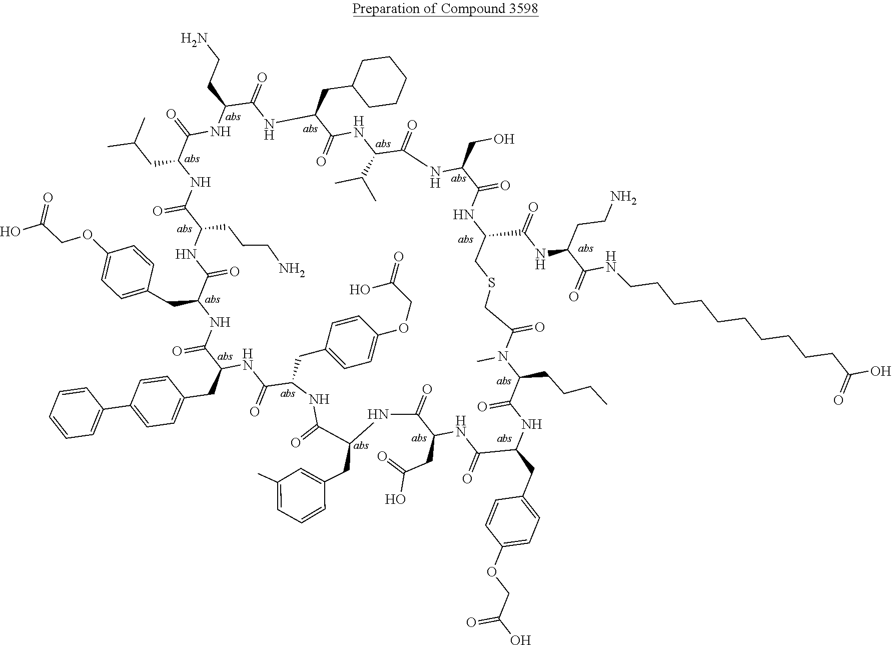 [SMILES: CCCC[C@H]1C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CO)C(=O)N[C@H](C(=O)N[C@@H](CCN)C(=O)NCCCCCCCCCCC(=O)O)CSCC(=O)N1C]

Compound 3598 was prepared on a 50 μmol scale. The yield of the product was 3.9 mg, and its estimated purity by LCMS analysis was 98%. Analysis condition A: Retention time=1.57 min; ESI-MS(+) m/z [M+2H]²⁺: 1201.2.

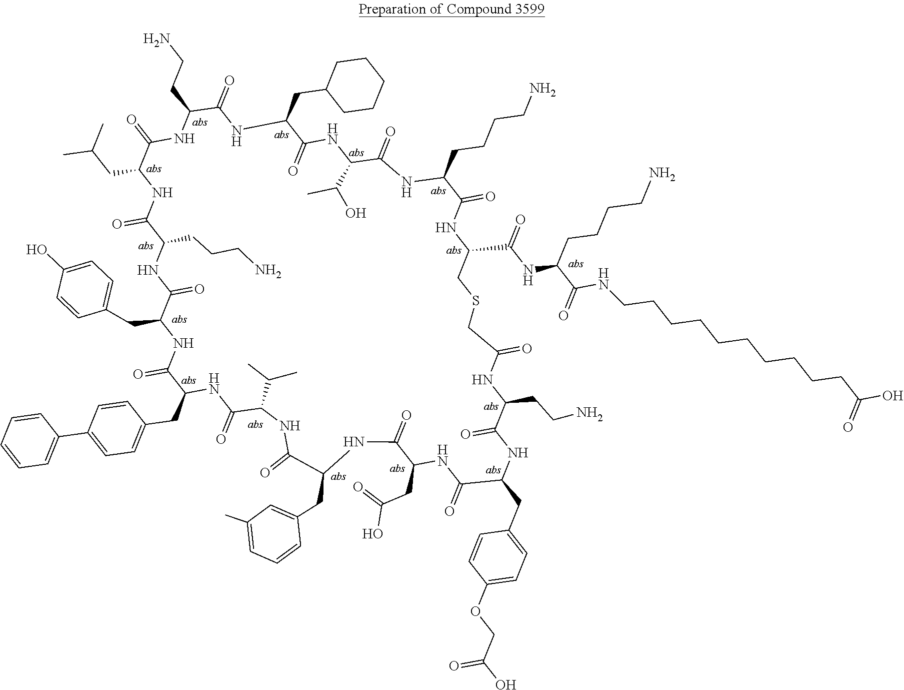 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCCCN)C(=O)NCCCCCCCCCCC(=O)O)NC(=O)[C@H](CCCCN)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3599 was prepared on a 50 μmol scale. The yield of the product was 22.8 mg, and its estimated purity by LCMS analysis was 90.3%. Analysis condition B: Retention time=1.44 min; ESI-MS(+) m/z [M+3H]³⁺: 756.2.

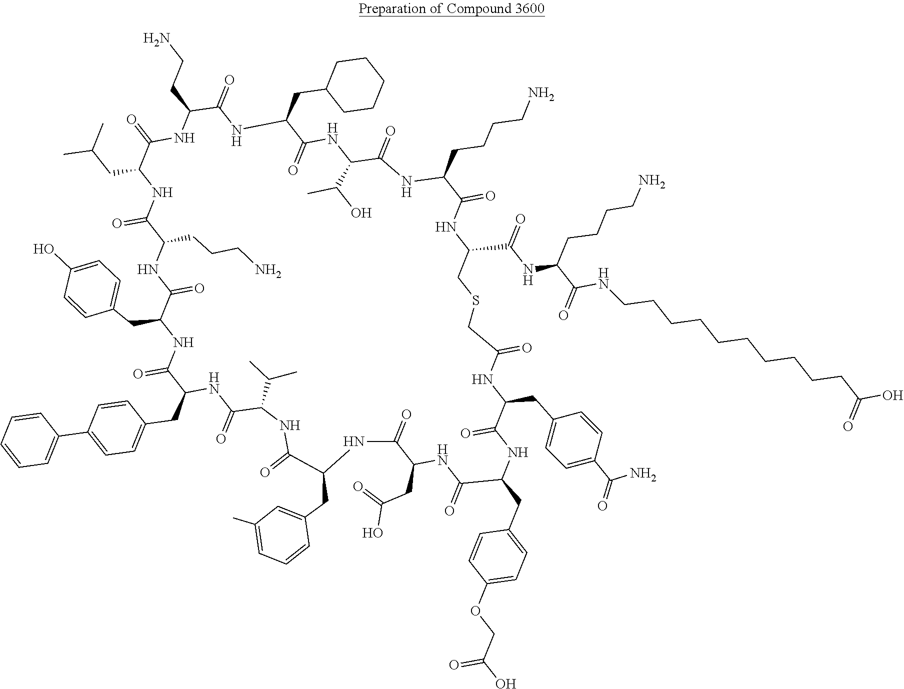 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCCCN)C(=O)NCCCCCCCCCCC(=O)O)NC(=O)[C@H](CCCCN)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3600 was prepared on a 50 μmol scale. The yield of the product was 18.5 mg, and its estimated purity by LCMS analysis was 98.7%. Analysis condition A: Retention time=1.57 min; ESI-MS(+) m/z [M+3H]³⁺: 786.3.

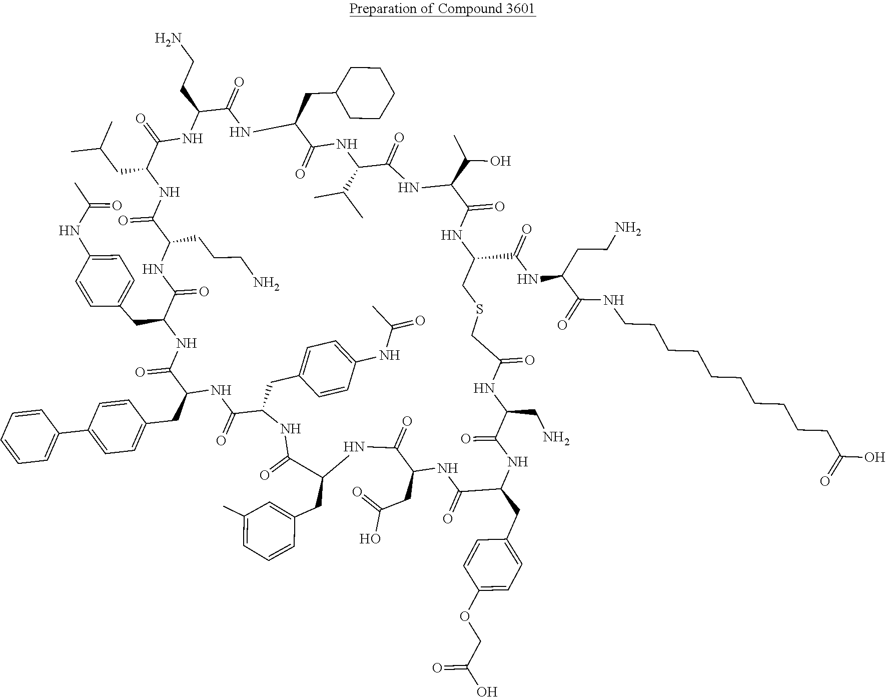 [SMILES: CC(=O)Nc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(NC(C)=O)cc3)NC(=O)[C@H](Cc3ccc(-c4ccccc4)cc3)NC2=O)cc1]

Compound 3601 was prepared on a 50 μmol scale. The yield of the product was 9.8 mg, and its estimated purity by LCMS analysis was 92.8%. Analysis condition A: Retention time=1.61 min; ESI-MS(+) m/z [M+2H]²⁺: 1170.8.

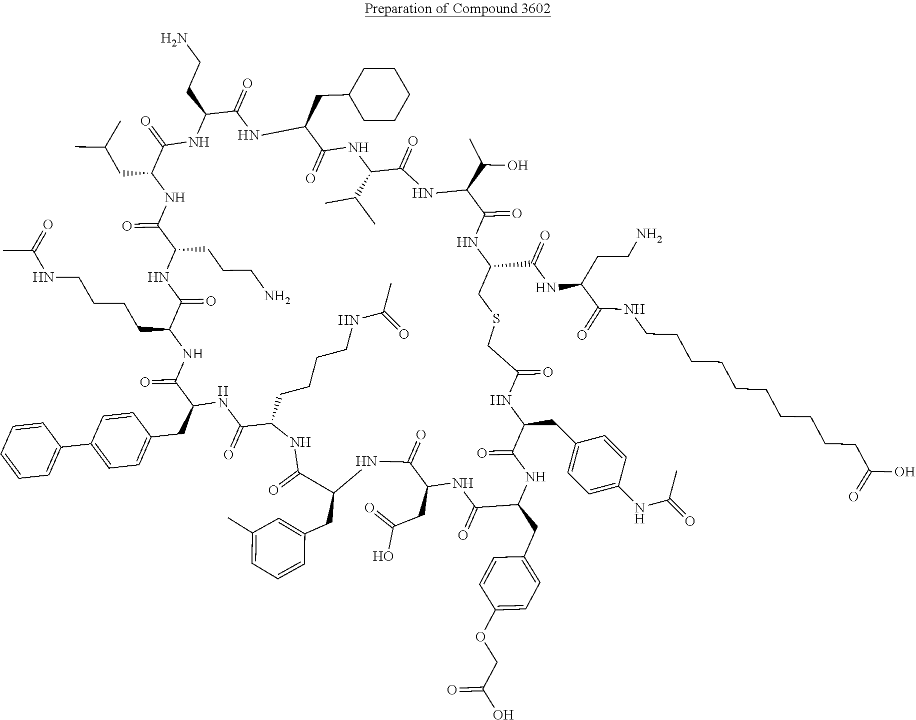 [SMILES: CC(=O)NCCCC[C@@H]1NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(NC(C)=O)cc2)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCCNC(C)=O)NC(=O)[C@H](Cc2ccc(-c3ccccc3)cc2)NC1=O]

Compound 3602 was prepared on a 50 μmol scale. The yield of the product was 3.3 mg, and its estimated purity by LCMS analysis was 96.3%. Analysis condition B: Retention time=1.67 min; ESI-MS(+) m/z [M+2H]²⁺: 1196.

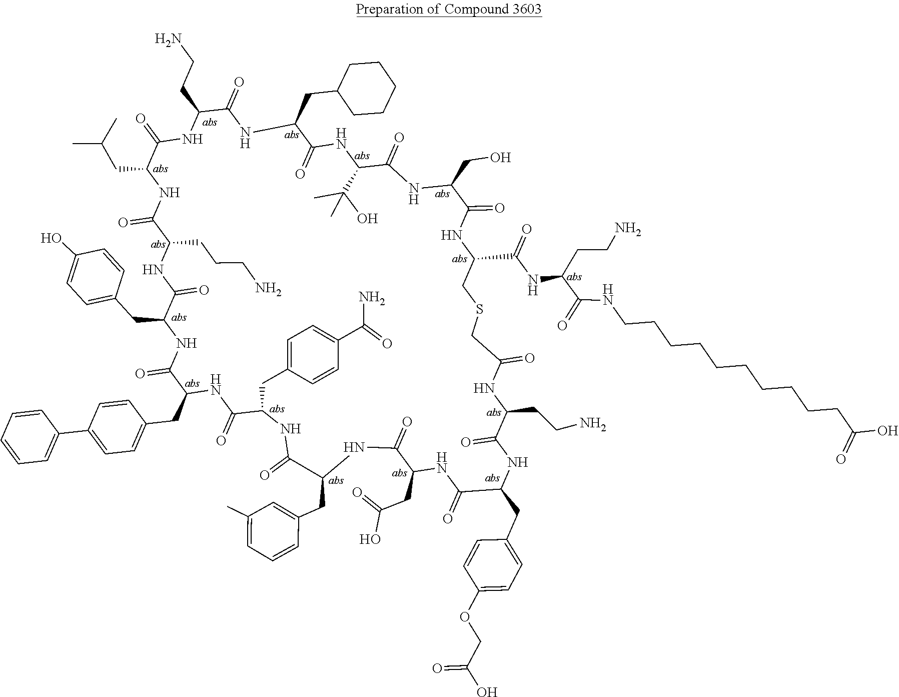 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 3603 was prepared on a 50 μmol scale. The yield of the product was 45.4 mg, and its estimated purity by LCMS analysis was 97.6%. Analysis condition A: Retention time=1.54 min; ESI-MS(+) m/z [M+3H]³⁺: 768.2.

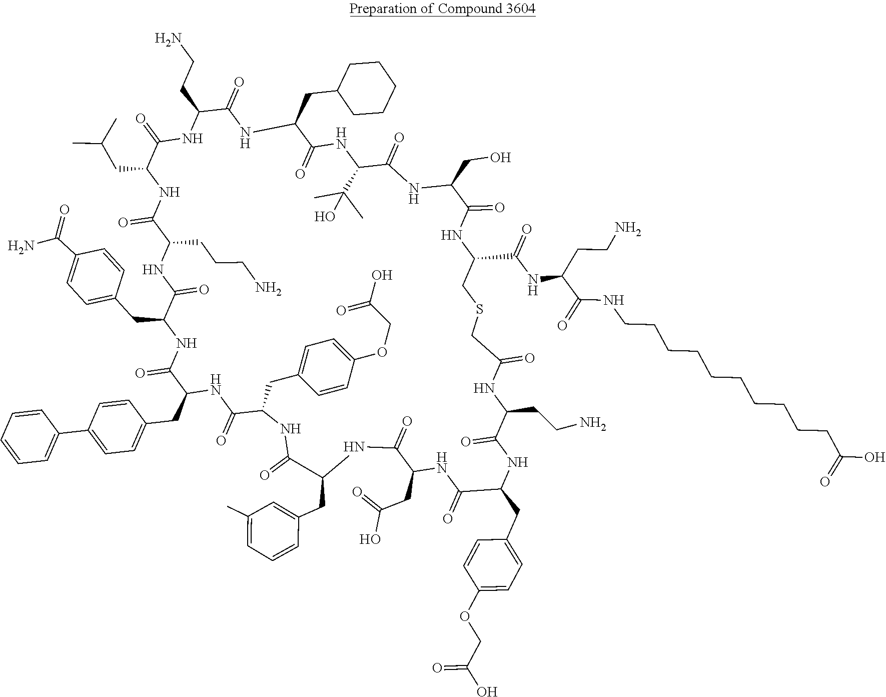 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(N)=O)cc3)NC(=O)[C@H](Cc3ccc(-c4ccccc4)cc3)NC(=O)[C@H](Cc3ccc(OCC(=O)O)cc3)NC2=O)c1]

Compound 3604 was prepared on a 50 μmol scale. The yield of the product was 8.1 mg, and its estimated purity by LCMS analysis was 95.6%. Analysis condition A: Retention time=1.51 min; ESI-MS(+) m/z [M+3H]³⁺: 787.4.

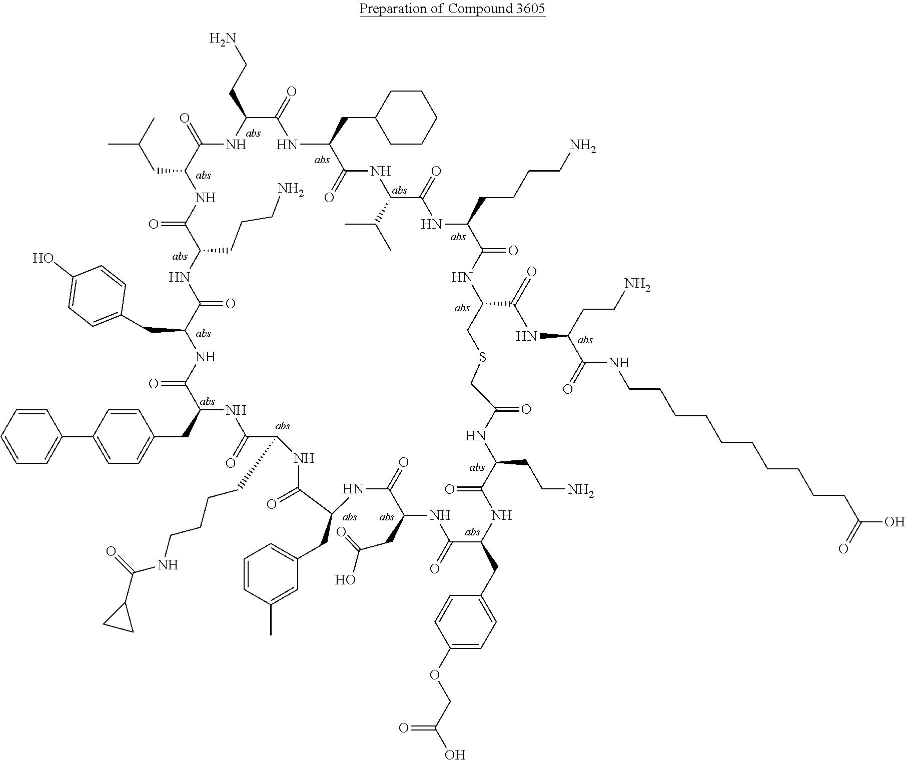 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CCCCNC(=O)C3CC3)NC2=O)c1]

Compound 3605 was prepared on a 50 μmol scale. The yield of the product was 16.1 mg, and its estimated purity by LCMS analysis was 95.9%. Analysis condition A: Retention time=1.49 min; ESI-MS(+) m/z [M+2H]²⁺: 1181.2.

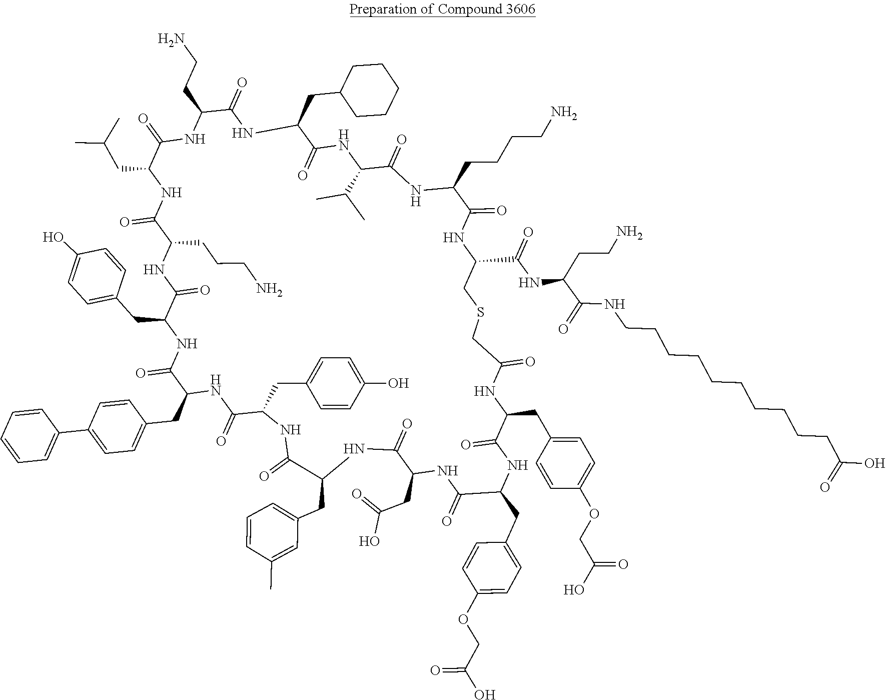 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 3606 was prepared on a 50 μmol scale. The yield of the product was 10.5 mg, and its estimated purity by LCMS analysis was 94.6%. Analysis condition A: Retention time=1.55 min; ESI-MS(+) m/z [M+2H]²⁺: 1225.2.

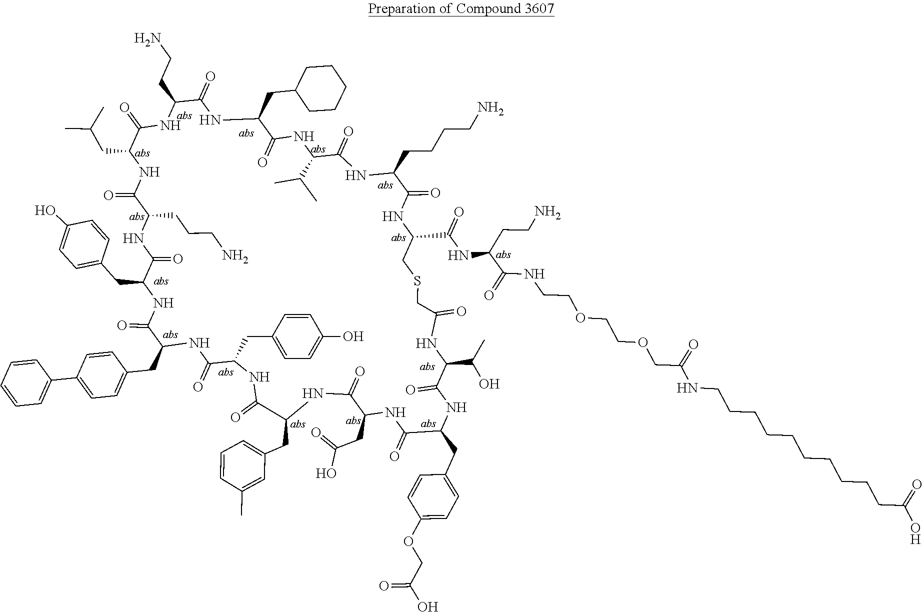 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 3607 was prepared on a 50 μmol scale. The yield of the product was 12.4 mg, and its estimated purity by LCMS analysis was 100%. Analysis condition B: Retention time=1.55 min; ESI-MS(+) m/z [M+3H]³⁺: 816.2.

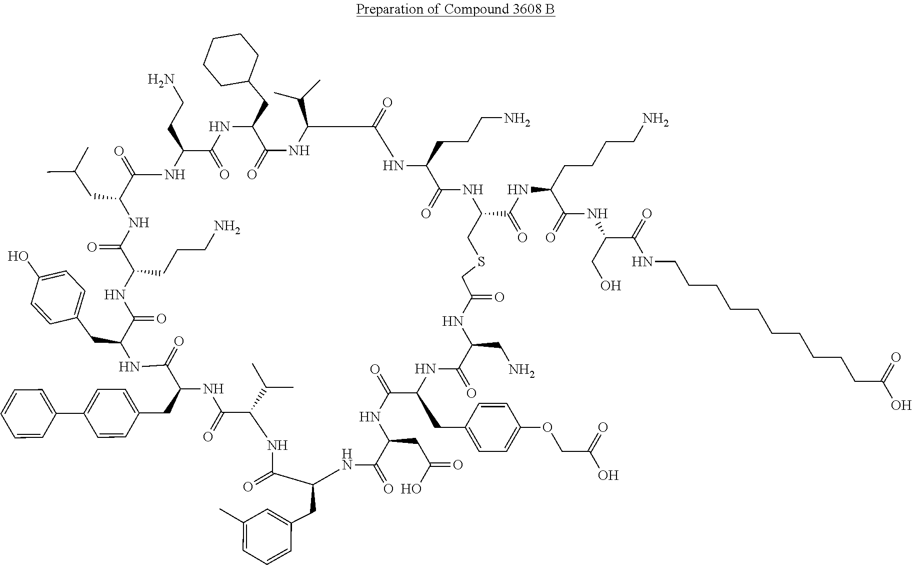 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCCN)C(=O)N[C@@H](CO)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3608 was prepared on a 50 μmol scale. The yield of the product was 5.2 mg, and its estimated purity by LCMS analysis was 100%. Analysis condition A: Retention time=1.56 min; ESI-MS(+) m/z [M+2H]²⁺: 1162.

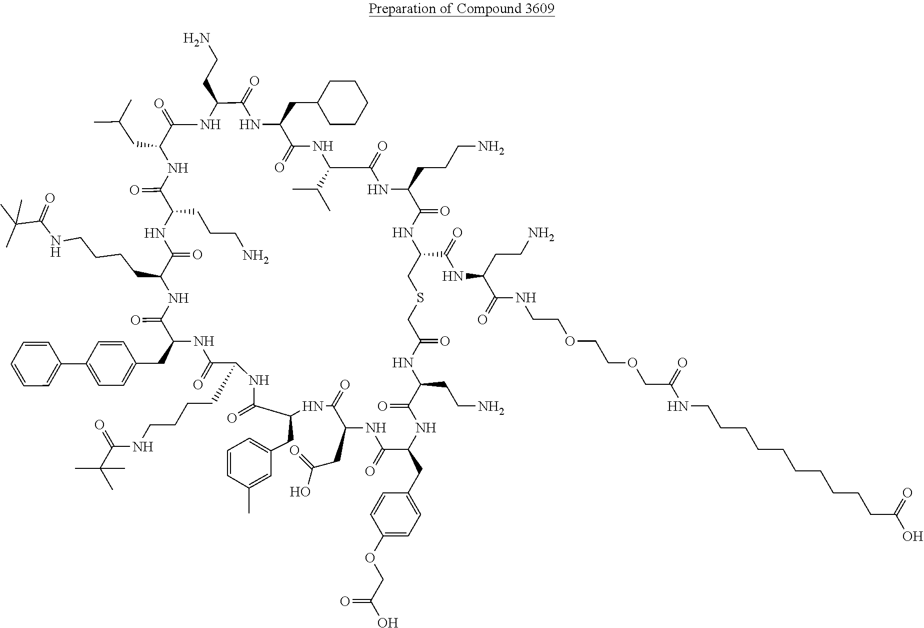 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCCNC(=O)C(C)(C)C)NC(=O)[C@H](Cc3ccc(-c4ccccc4)cc3)NC(=O)[C@H](CCCCNC(=O)C(C)(C)C)NC2=O)c1]

Compound 3609 was prepared on a 50 μmol scale. The yield of the product was 19.9 mg, and its estimated purity by LCMS analysis was 100%. Analysis condition A: Retention time=1.86 min; ESI-MS(+) m/z [M+3H]³⁺: 844.

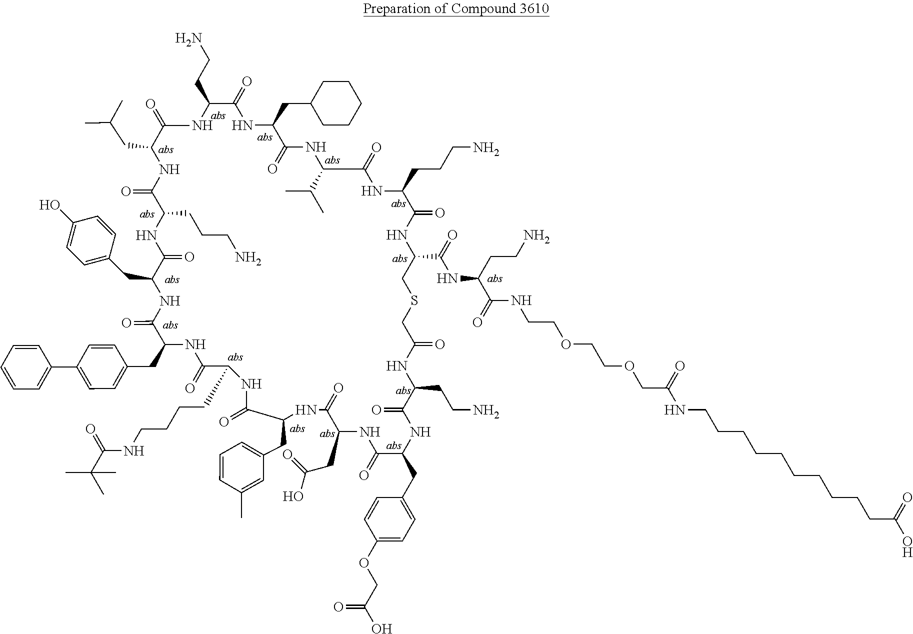 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CCCCNC(=O)C(C)(C)C)NC2=O)c1]

Compound 3610 was prepared on a 50 μmol scale. The yield of the product was 32.6 mg, and its estimated purity by LCMS analysis was 94.2%. Analysis condition A: Retention time=1.57, 1.63 min; ESI-MS(+) m/z [M+3H]³⁺: 827.52, 827.08.

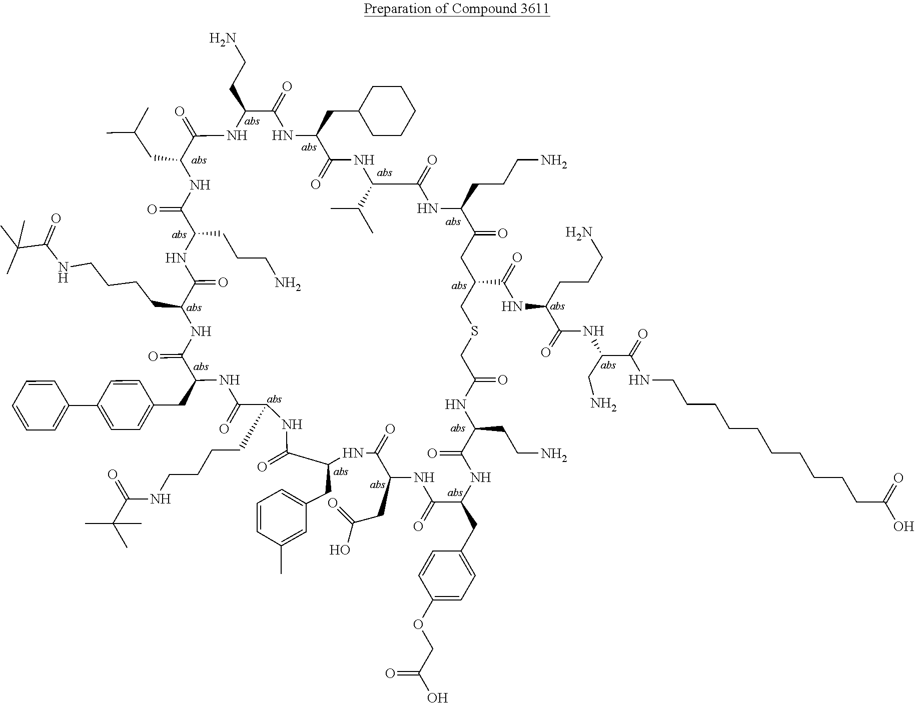

Compound 3611 was prepared on a 50 μmol scale. The yield of the product was 19.7 mg, and its estimated purity by LCMS analysis was 95%. Analysis condition A: Retention time=1.65 min; ESI-MS(+) m/z [M+2H]²⁺: 1243.

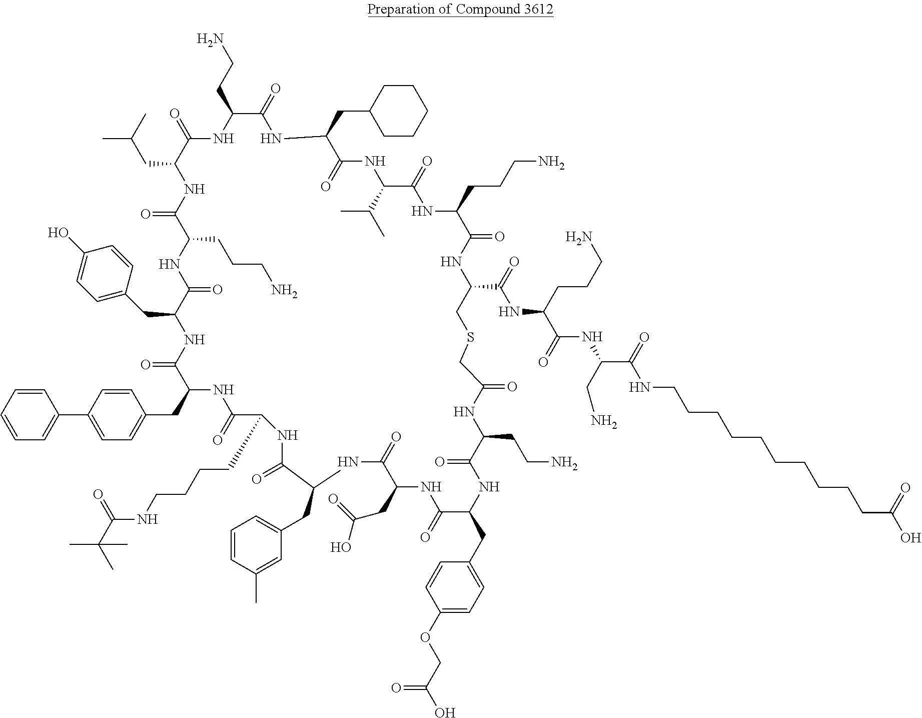 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCCN)C(=O)N[C@@H](CN)C(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CCCCNC(=O)C(C)(C)C)NC2=O)c1]

Compound 3612 was prepared on a 50 μmol scale. The yield of the product was 10.7 mg, and its estimated purity by LCMS analysis was 100%. Analysis condition A: Retention time=1.66 min; ESI-MS(+) m/z [M+3H]³⁺: 812.3.

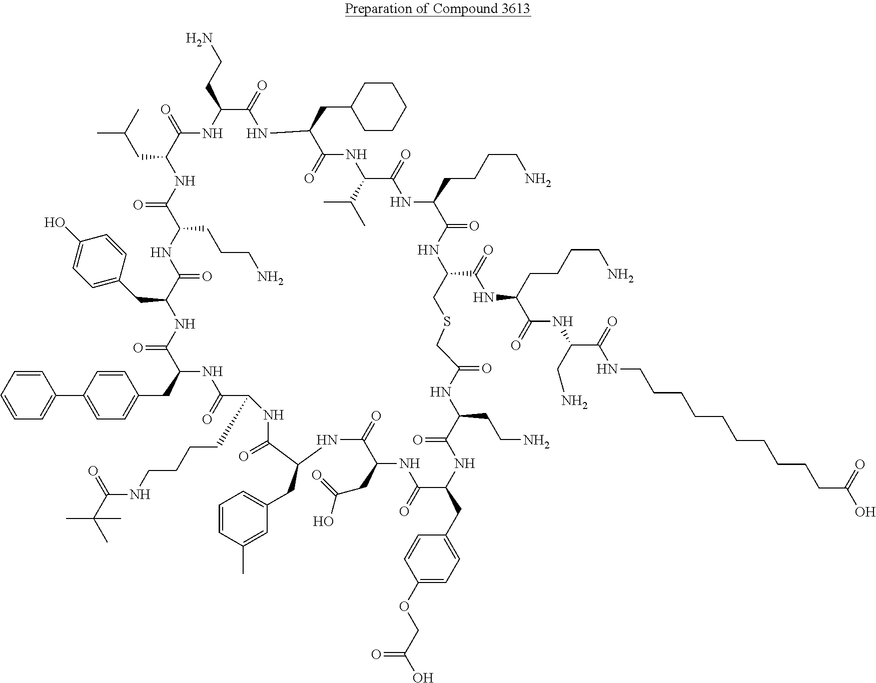 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCCCN)C(=O)N[C@@H](CN)C(=O)NCCCCCCCCCCC(=O)O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](CCCCNC(=O)C(C)(C)C)NC2=O)c1]

Compound 3613 was prepared on a 50 μmol scale. The yield of the product was 2.2 mg, and its estimated purity by LCMS analysis was 100%. Analysis condition A: Retention time=1.74 min; ESI-MS(+) m/z [M+2H]²⁺: 1232.1.

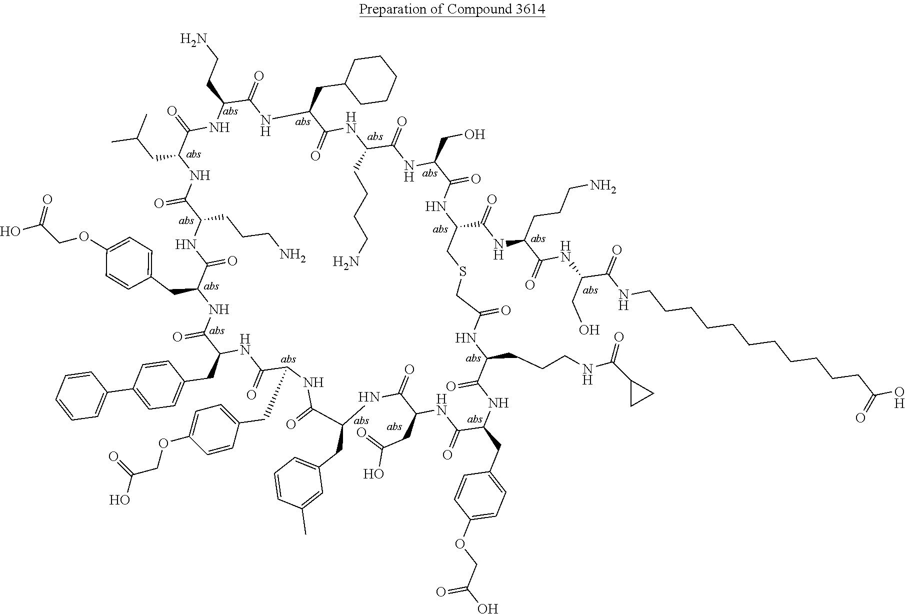 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=O)C3CC3)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](CCCCN)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3614 was prepared on a 50 μmol scale. The yield of the product was 16.5 mg, and its estimated purity by LCMS analysis was 95.6%. Analysis condition B: Retention time=1.56 min; ESI-MS(+) m/z [M+2H]²⁺: 1303.5.

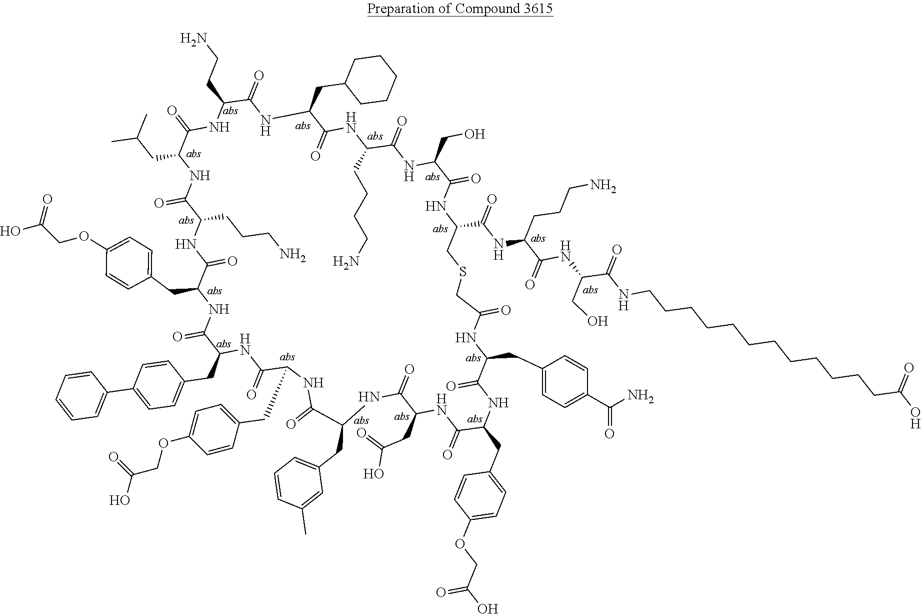 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCCN)C(=O)N[C@@H](CO)C(=O)NCCCCCCCCCCCCC(=O)O)NC(=O)[C@H](CO)NC(=O)[C@H](CCCCN)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3615 was prepared on a 50 μmol scale. The yield of the product was 2.2 mg, and its estimated purity by LCMS analysis was 100%. Analysis condition B: Retention time=1.55 min; ESI-MS(+) m/z [M+2H]²⁺: 870.4.

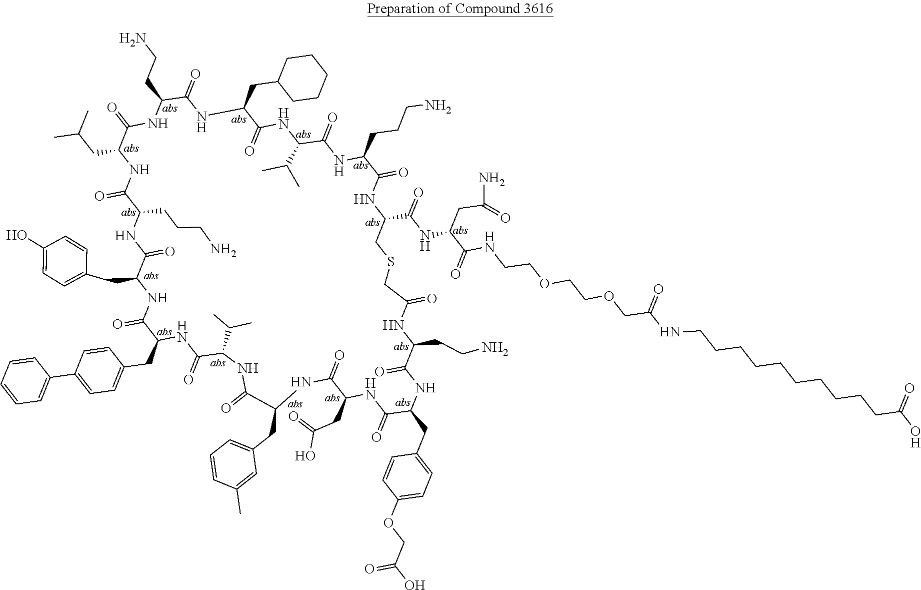 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@H](CC(N)=O)C(=O)NCCOCCOCC(=O)NCCCCCCCCCCC(=O)O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 3616 was prepared on a 50 μmol scale. The yield of the product was 23.4 mg, and its estimated purity by LCMS analysis was 98.2%. Analysis condition A: Retention time=1.56 min; ESI-MS(+) m/z [M+2H]²⁺: 1190.9.

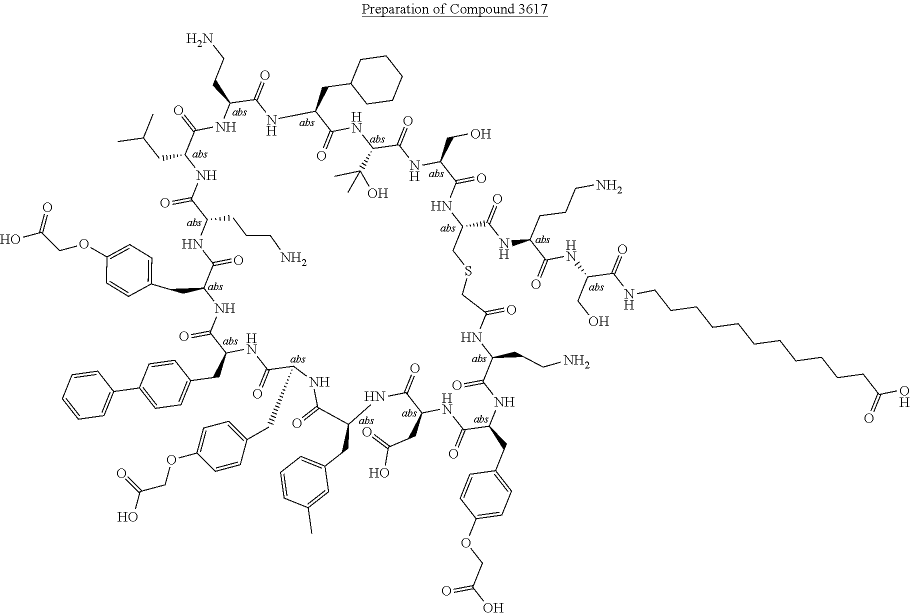 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3617 was prepared on a 50 μmol scale. The yield of the product was 4.4 mg, and its estimated purity by LCMS analysis was 100%. Analysis condition A: Retention time=1.5 min; ESI-MS(+) m/z [M+3H]³⁺: 836.1.

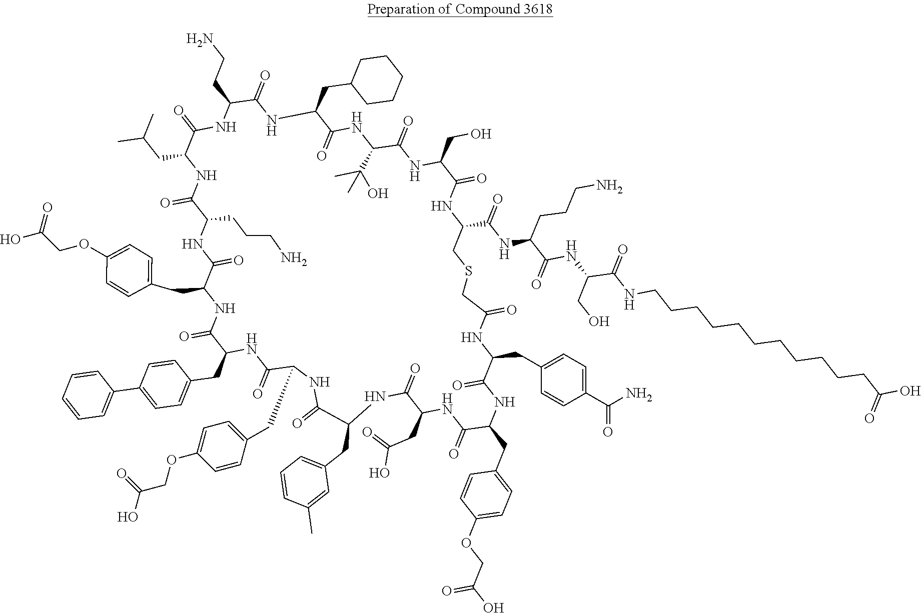 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCCN)C(=O)N[C@@H](CO)C(=O)NCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3618 was prepared on a 50 μmol scale. The yield of the product was 26.6 mg, and its estimated purity by LCMS analysis was 96.6%. Analysis condition B: Retention time=1.66 min; ESI-MS(+) m/z [M+3H]³⁺: 866.1.

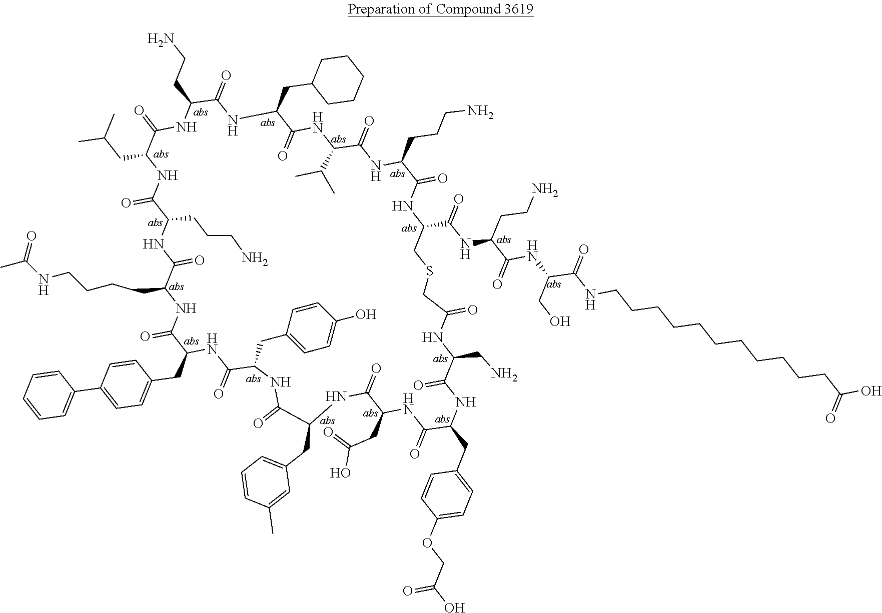 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)N[C@@H](CO)C(=O)NCCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3619 was prepared on a 50 μmol scale. The yield of the product was 14.1 mg, and its estimated purity by LCMS analysis was 96%. Analysis condition B: Retention time=1.47 min; ESI-MS(+) m/z [M+3H]³⁺: 794.

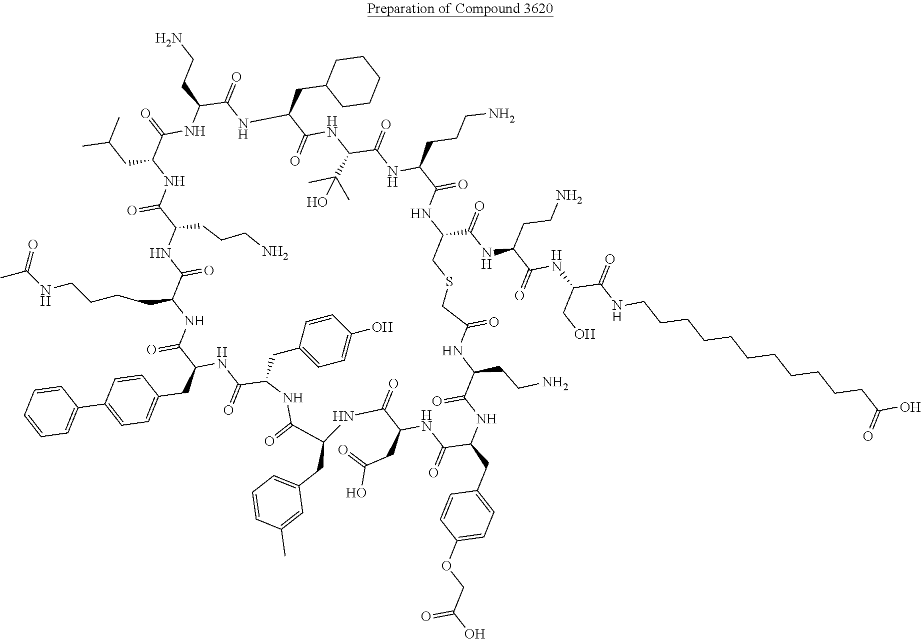 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)N[C@@H](CO)C(=O)NCCCCCCCCCCCC(=O)O)NC(=O)[C@H](CCCN)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC1=O]

Compound 3620 was prepared on a 50 μmol scale. The yield of the product was 34 mg, and its estimated purity by LCMS analysis was 95.1%. Analysis condition B: Retention time=1.56 min; ESI-MS(+) m/z [M+3H]³⁺: 804.2.

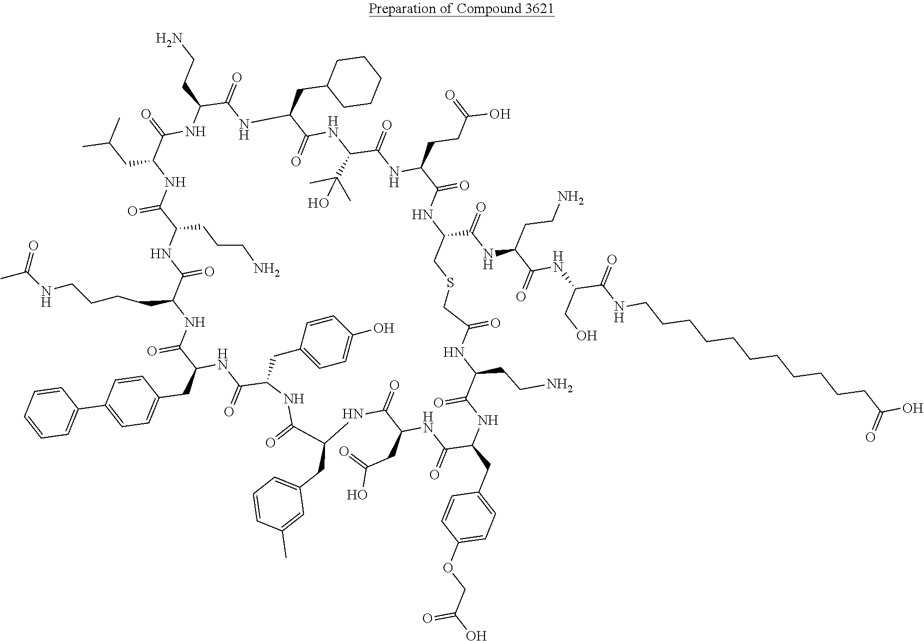 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)N[C@@H](CO)C(=O)NCCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC1=O]

Compound 3621 was prepared on a 50 μmol scale. The yield of the product was 11.8 mg, and its estimated purity by LCMS analysis was 98.3%. Analysis condition B: Retention time=1.56 min; ESI-MS(+) m/z [M+2H]²⁺: 1213.2.

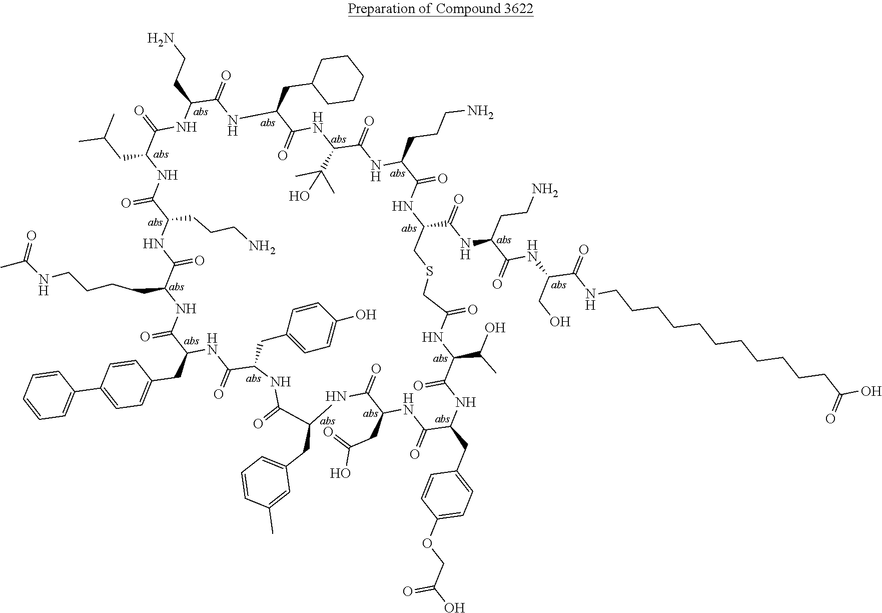 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)N[C@@H](CO)C(=O)NCCCCCCCCCCCC(=O)O)NC(=O)[C@H](CCCN)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC1=O]

Compound 3622 was prepared on a 50 μmol scale. The yield of the product was 18.6 mg, and its estimated purity by LCMS analysis was 95.3%. Analysis condition A: Retention time-1.67 min; ESI-MS(+) m/z [M+3H]³⁺: 804.2.

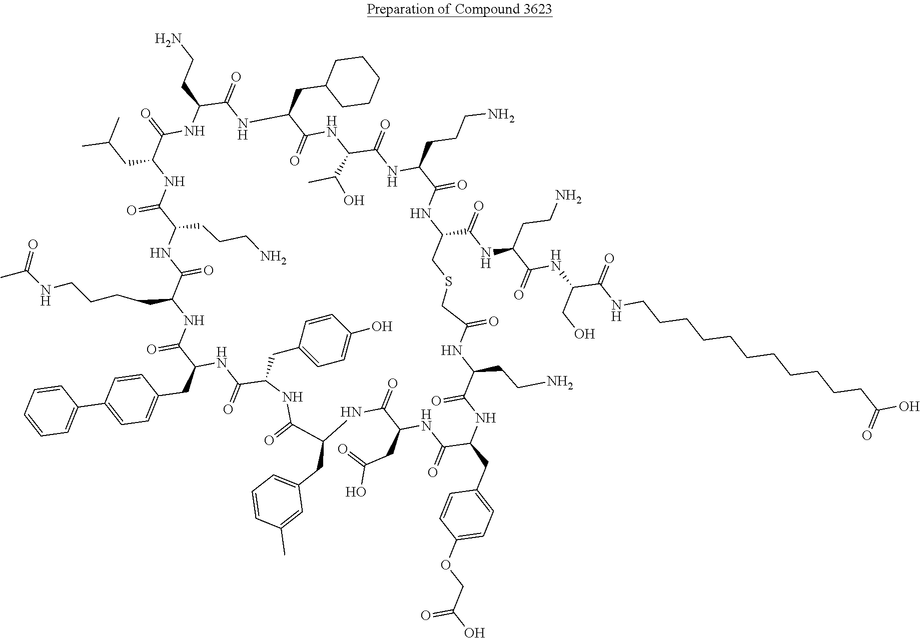 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)N[C@@H](CO)C(=O)NCCCCCCCCCCCC(=O)O)NC(=O)[C@H](CCCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC1=O]

Compound 3623 was prepared on a 50 μmol scale. The yield of the product was 15.1 mg, and its estimated purity by LCMS analysis was 93.3%. Analysis condition B: Retention time=1.47 min; ESI-MS(+) m/z [M+3H]³⁺: 799.1.

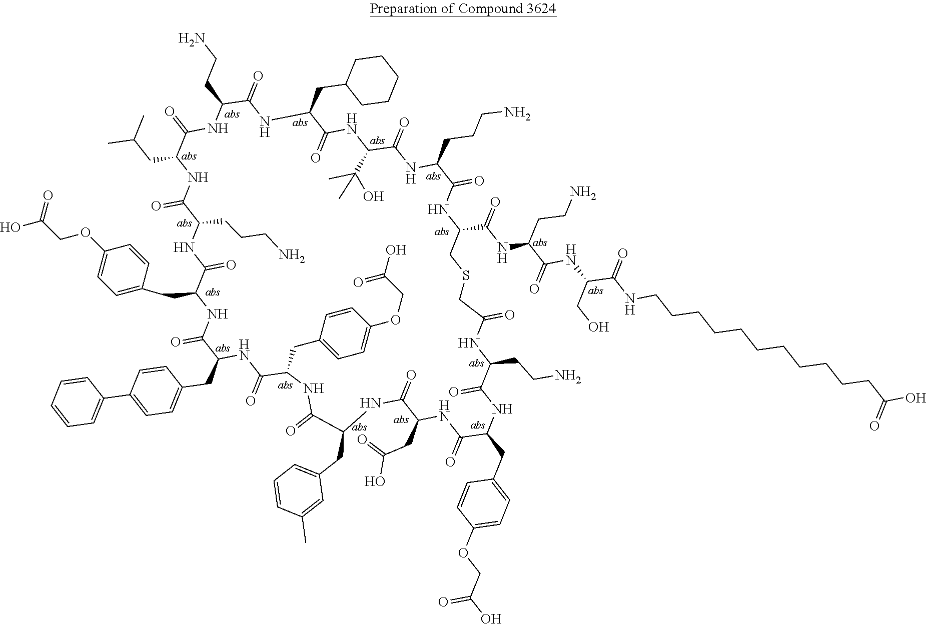 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)N[C@@H](CO)C(=O)NCCCCCCCCCCCC(=O)O)NC(=O)[C@H](CCCN)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3624 was prepared on a 50 μmol scale. The yield of the product was 19.1 mg, and its estimated purity by LCMS analysis was 98.2%. Analysis condition A: Retention time=1.56 min; ESI-MS(+) m/z [M+2H]²⁺: 1260.

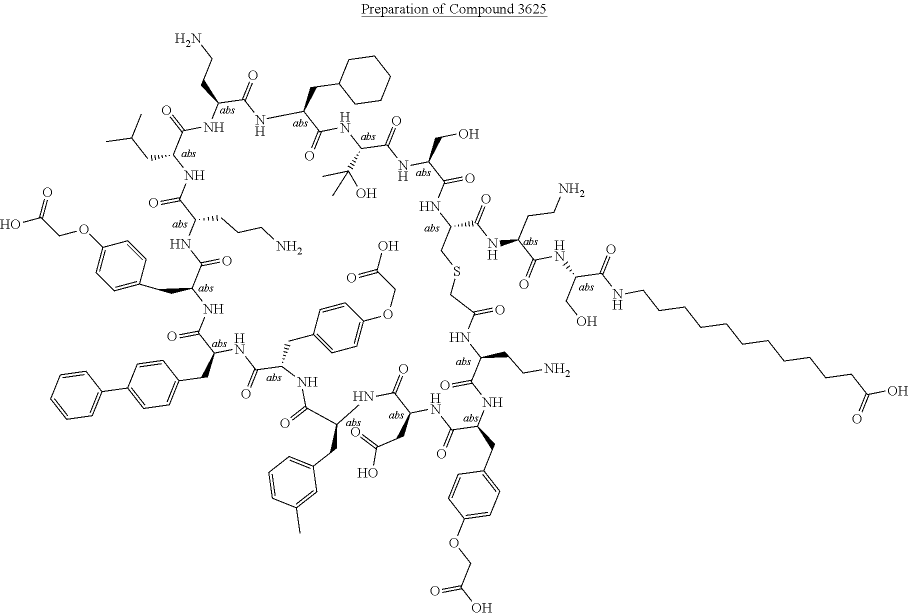 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)N[C@@H](CO)C(=O)NCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3625 was prepared on a 50 μmol scale. The yield of the product was 25.7 mg, and its estimated purity by LCMS analysis was 86.3%. Analysis condition A: Retention time=1.58 min; ESI-MS(+) m/z [M+3H]³⁺: 831.2.

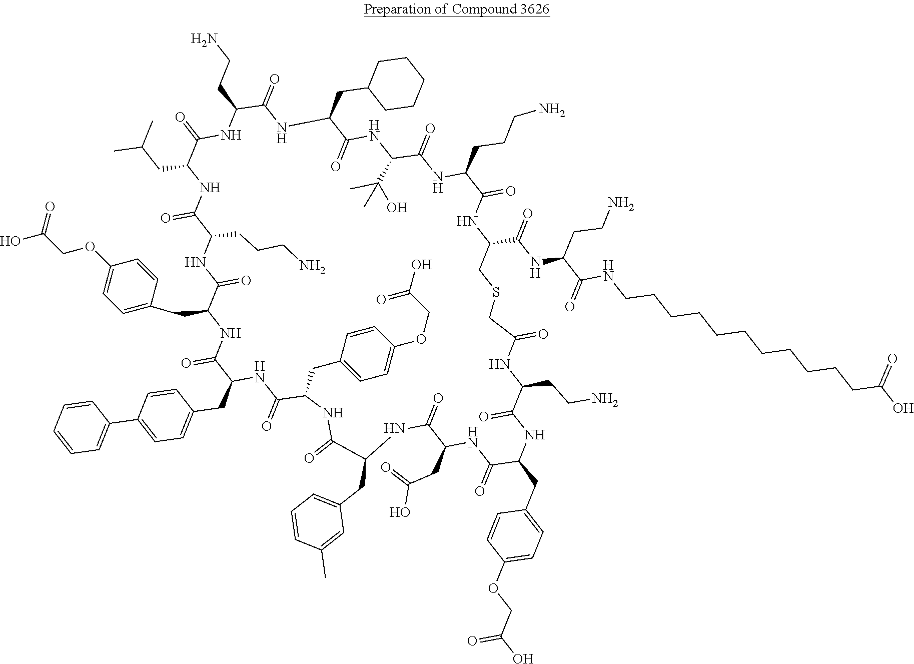 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCCC(=O)O)NC(=O)[C@H](CCCN)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3626 was prepared on a 50 μmol scale. The yield of the product was 5.4 mg, and its estimated purity by LCMS analysis was 98.9%. Analysis condition A: Retention time=1.56 min; ESI-MS(+) m/z [M+3H]³⁺: 811.

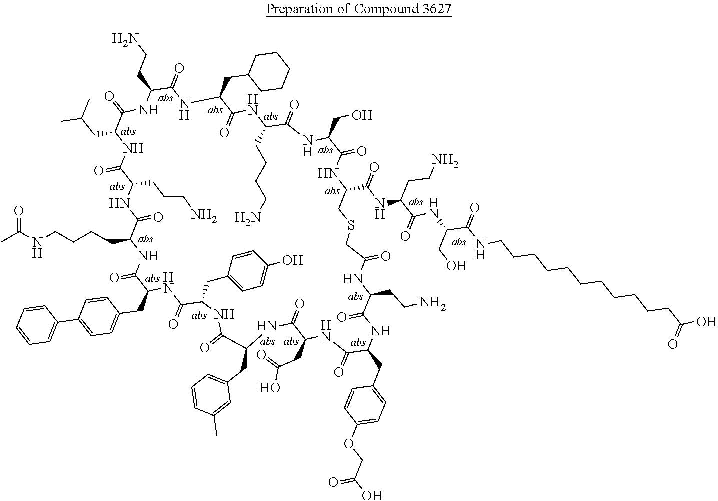 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)N[C@@H](CO)C(=O)NCCCCCCCCCCCC(=O)O)NC(=O)[C@H](CO)NC(=O)[C@H](CCCCN)NC(=O)[C@H](CC2CCCCC2)NC(=O)[C@H](CCN)NC(=O)[C@@H](CC(C)C)NC(=O)[C@H](CCCN)NC1=O]

Compound 3627 was prepared on a 50 μmol scale. The yield of the product was 18.8 mg, and its estimated purity by LCMS analysis was 91%. Analysis condition B: Retention time=1.47 min; ESI-MS(+) m/z [M+3H]³⁺: 799.1.

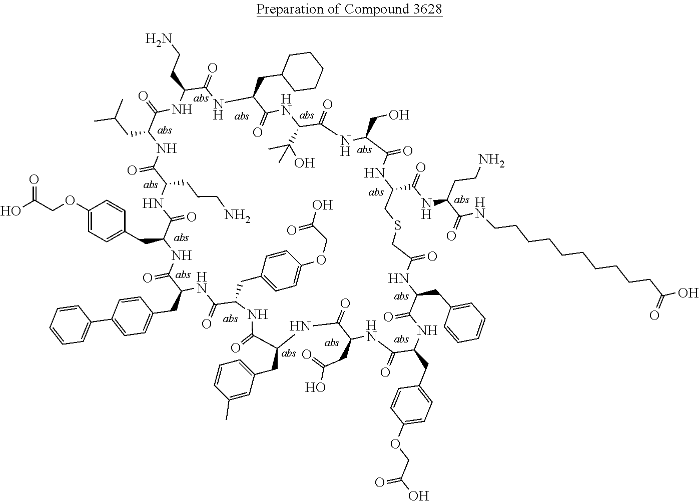 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccccc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3628 was prepared on a 50 μmol scale. The yield of the product was 5 mg, and its estimated purity by LCMS analysis was 98.7%. Analysis condition B: Retention time=1.81 min; ESI-MS(+) m/z [M+3H]³⁺: 813.4.

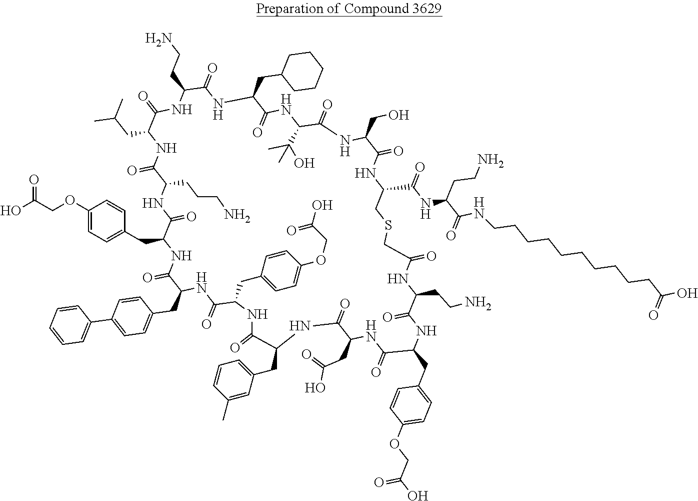 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3629 was prepared on a 50 μmol scale. The yield of the product was 2.8 mg, and its estimated purity by LCMS analysis was 99%. Analysis condition B: Retention time=1.5 min; ESI-MS(+) m/z [M+3H]³⁺: 797.3.

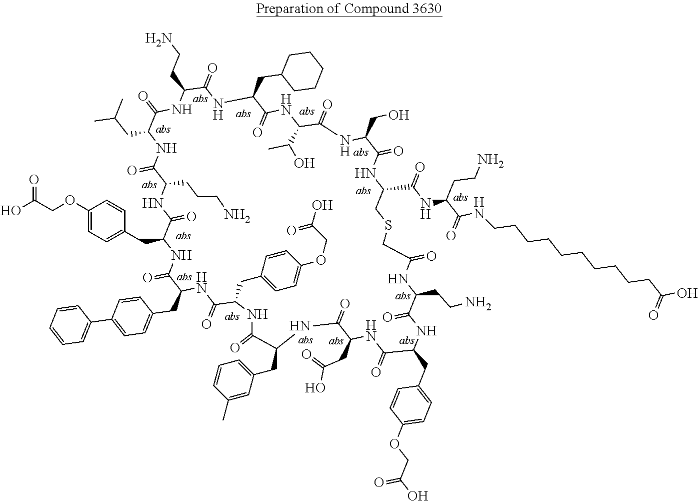 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3630 was prepared on a 50 μmol scale. The yield of the product was 3.6 mg, and its estimated purity by LCMS analysis was 95.7%. Analysis condition A: Retention time=1.52 min; ESI-MS(+) m/z [M+2H]²⁺: 1189.2.

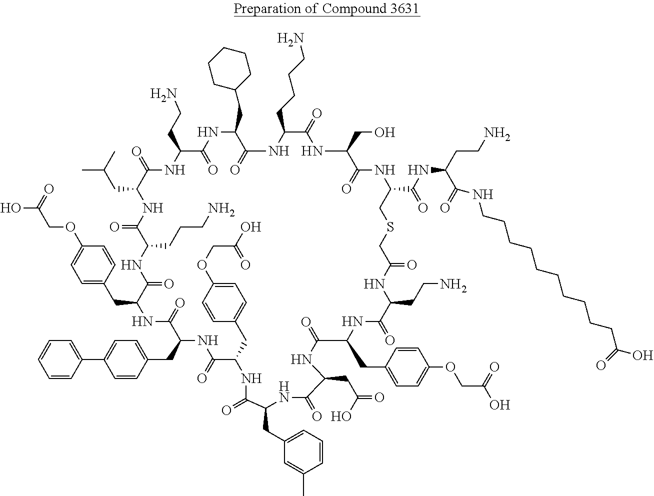 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](CCCCN)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3631 was prepared on a 50 μmol scale. The yield of the product was 1.8 mg, and its estimated purity by LCMS analysis was 95.4%. Analysis condition A: Retention time=1.57 min; ESI-MS(+) m/z [M+2H]²⁺: 1202.3.

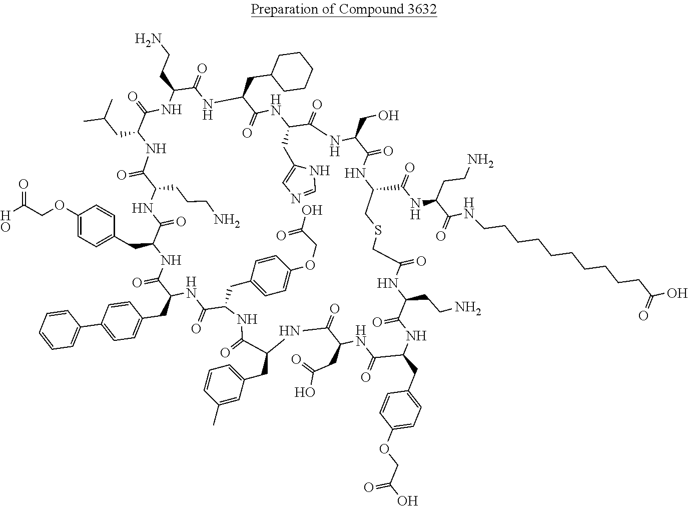 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3632 was prepared on a 50 μmol scale. The yield of the product was 10.8 mg, and its estimated purity by LCMS analysis was 96.9%. Analysis condition B: Retention time=1.59 min; ESI-MS(+) m/z [M+2H]²⁺: 1207.2.

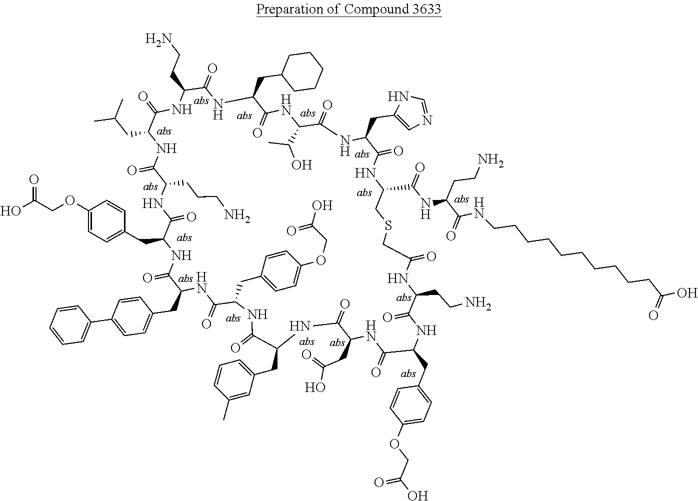 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3633 was prepared on a 50 μmol scale. The yield of the product was 25.1 mg, and its estimated purity by LCMS analysis was 96.5%. Analysis condition A: Retention time=1.41, 1.44 min; ESI-MS(+) m/z [M+2H]²⁺: 1214.01, 1214.01.

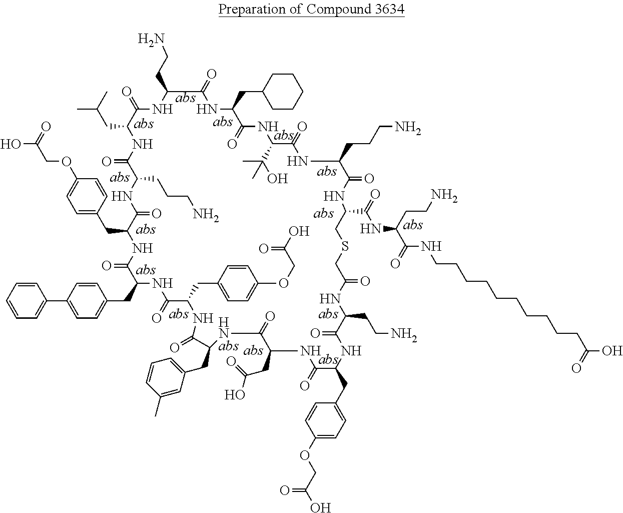 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CCCN)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3634 was prepared on a 50 μmol scale. The yield of the product was 8.1 mg, and its estimated purity by LCMS analysis was 100%. Analysis condition A: Retention time=1.48 min; ESI-MS(+) m/z [M+2H]²⁺: 1209.3.

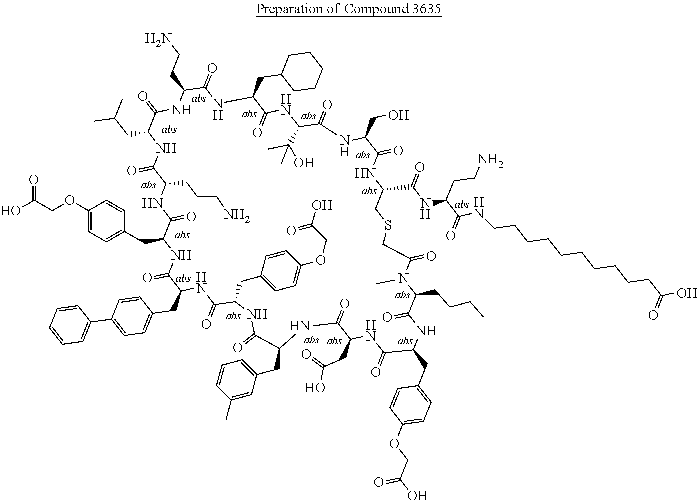 [SMILES: CCCC[C@H]1C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N[C@H](C(=O)N[C@@H](CCN)C(=O)NCCCCCCCCCCC(=O)O)CSCC(=O)N1C]

Compound 3635 was prepared on a 50 μmol scale. The yield of the product was 29.7 mg, and its estimated purity by LCMS analysis was 95.6%. Analysis condition A: Retention time=1.62 min; ESI-MS(+) m/z [M+2H]²⁺: 1209.4.

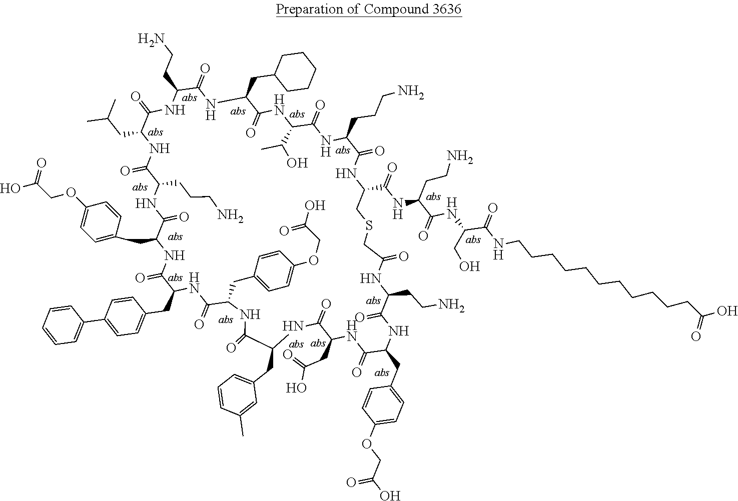 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)N[C@@H](CO)C(=O)NCCCCCCCCCCCC(=O)O)NC(=O)[C@H](CCCN)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3636 was prepared on a 50 μmol scale. The yield of the product was 5.9 mg, and its estimated purity by LCMS analysis was 100%. Analysis condition A: Retention time=1.47 min; ESI-MS(+) m/z [M+3H]³⁺: 836.1.

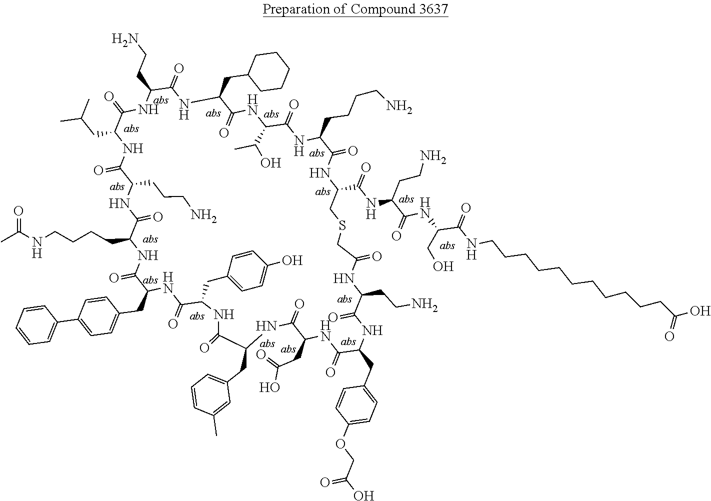 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)N[C@@H](CO)C(=O)NCCCCCCCCCCCC(=O)O)NC(=O)[C@H](CCCCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC1=O]

Compound 3637 was prepared on a 50 μmol scale. The yield of the product was 33.5 mg, and its estimated purity by LCMS analysis was 99.2%. Analysis condition A: Retention time=1.54 min; ESI-MS(+) m/z [M+3H]³⁺: 804.2.

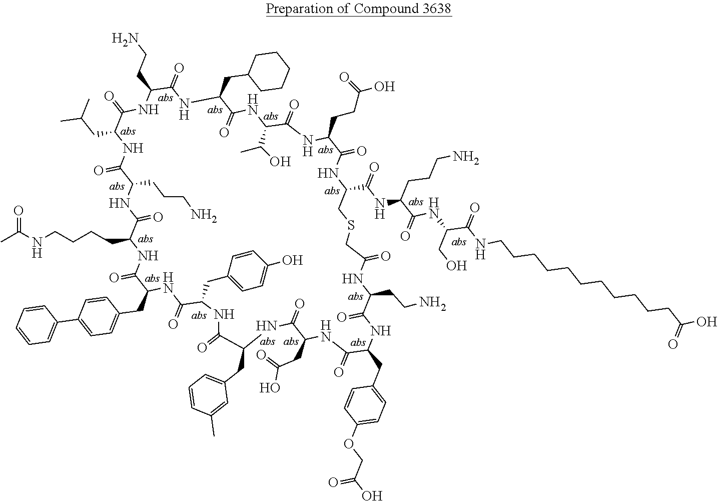 [SMILES: CC(=O)NCCCC[C@@H]1NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCCN)C(=O)N[C@@H](CO)C(=O)NCCCCCCCCCCCC(=O)O)NC(=O)[C@H](CCC(=O)O)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC1=O]

Compound 3638 was prepared on a 50 μmol scale. The yield of the product was 4.7 mg, and its estimated purity by LCMS analysis was 96.1%. Analysis condition B: Retention time=1.6 min; ESI-MS(+) m/z [M+3H]³⁺: 804.4.

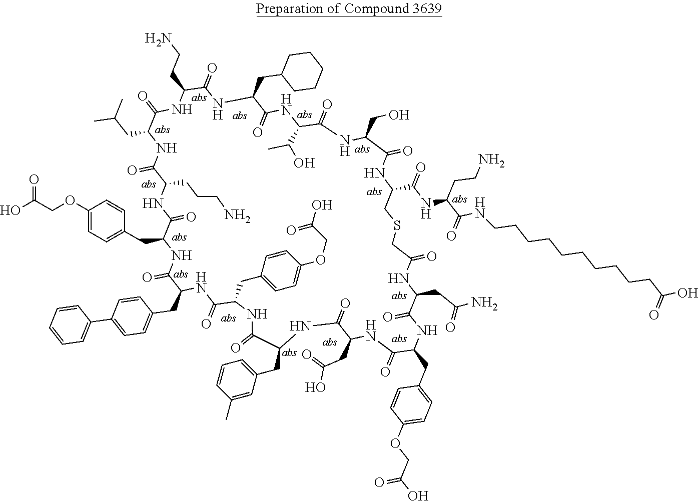 [SMILES: Cc1cccc(C[C@@H]2NC(=O)[C@H](CC(=O)O)NC(=O)[C@H](Cc3ccc(OCC(=O)O)cc3)NC(=O)[C@H](CC(N)=O)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3639 was prepared on a 50 μmol scale. The yield of the product was 3.4 mg, and its estimated purity by LCMS analysis was 100%. Analysis condition A: Retention time=1.41 min; ESI-MS(+) m/z [M+2H]²⁺: 1196.1.

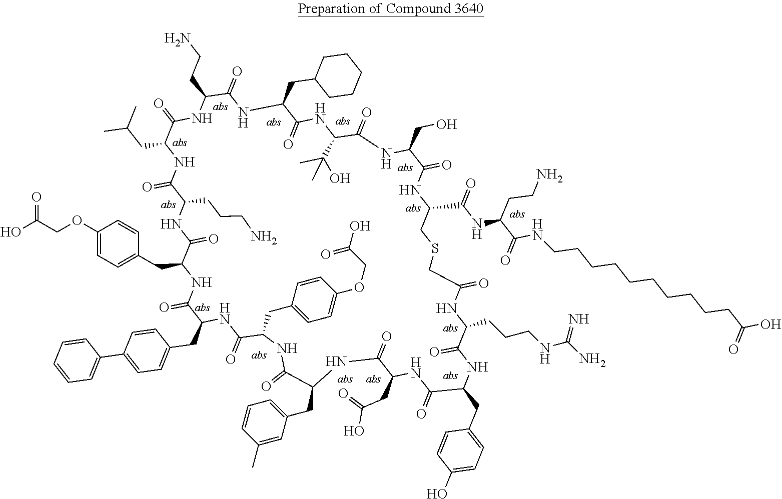 [SMILES: Cc1cccc(C[C@@H]2NC(=O)[C@H](CC(=O)O)NC(=O)[C@H](Cc3ccc(O)cc3)NC(=O)[C@@H](CCCNC(=N)N)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3640 was prepared on a 50 μmol scale. The yield of the product was 5.6 mg, and its estimated purity by LCMS analysis was 100%. Analysis condition A: Retention time=1.51 min; ESI-MS(+) m/z [M+2H]²⁺: 1195.1.

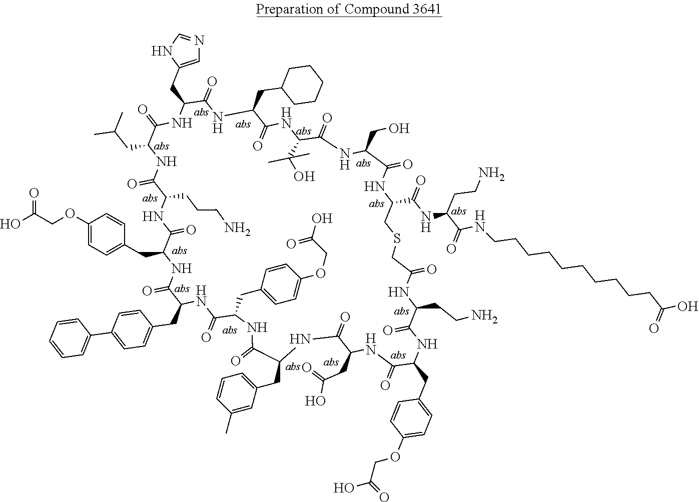 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3641 was prepared on a 50 μmol scale. The yield of the product was 10.1 mg, and its estimated purity by LCMS analysis was 100%. Analysis condition A: Retention time=1.6 min; ESI-MS(+) m/z [M+2H]²⁺: 1214.9.

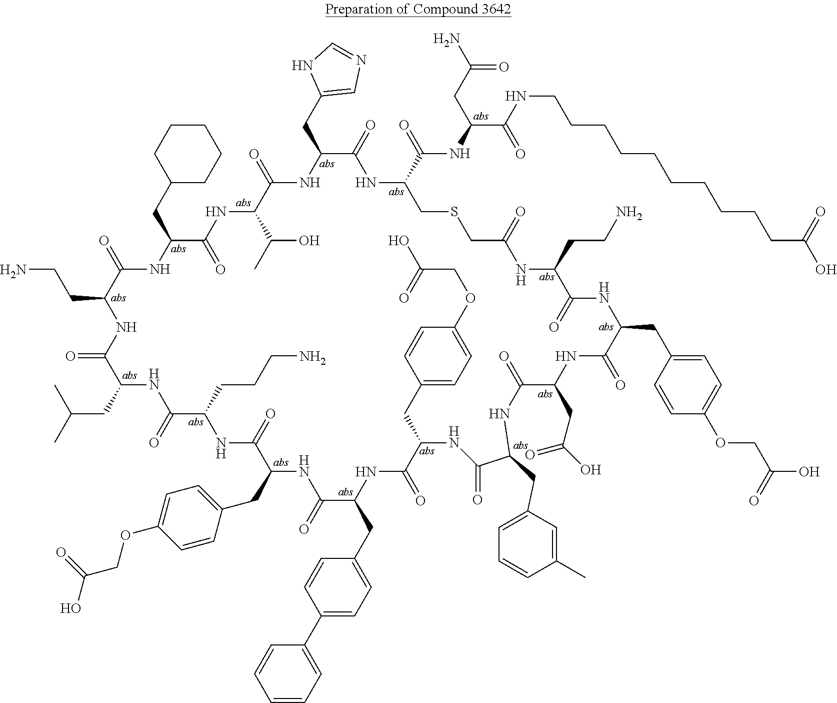 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CC(N)=O)C(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3642 was prepared on a 50 μmol scale. The yield of the product was 16.8 mg, and its estimated purity by LCMS analysis was 95.5%. Analysis condition B: Retention time=1.51 min; ESI-MS(+) m/z [M+2H]²⁺: 1221.2.

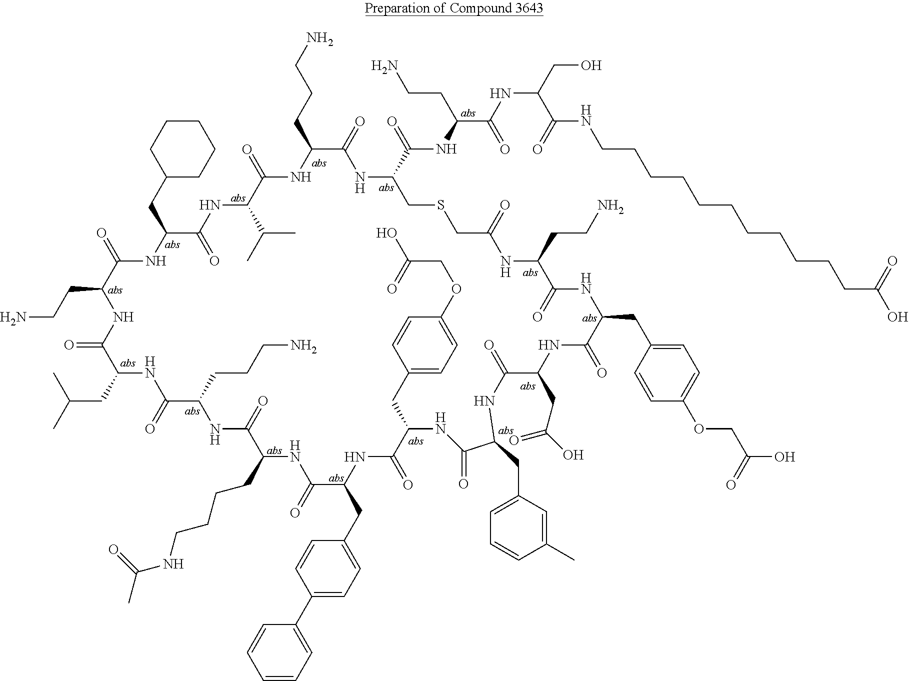 [SMILES: CC(=O)NCCCC[C@@H]1NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)NC(CO)C(=O)NCCCCCCCCCCCC(=O)O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 3643 was prepared on a 50 μmol scale. The yield of the product was 33.2 mg, and its estimated purity by LCMS analysis was 90%. Analysis condition B: Retention time=1.49 min; ESI-MS(+) m/z [M+2H]²⁺: 1187.

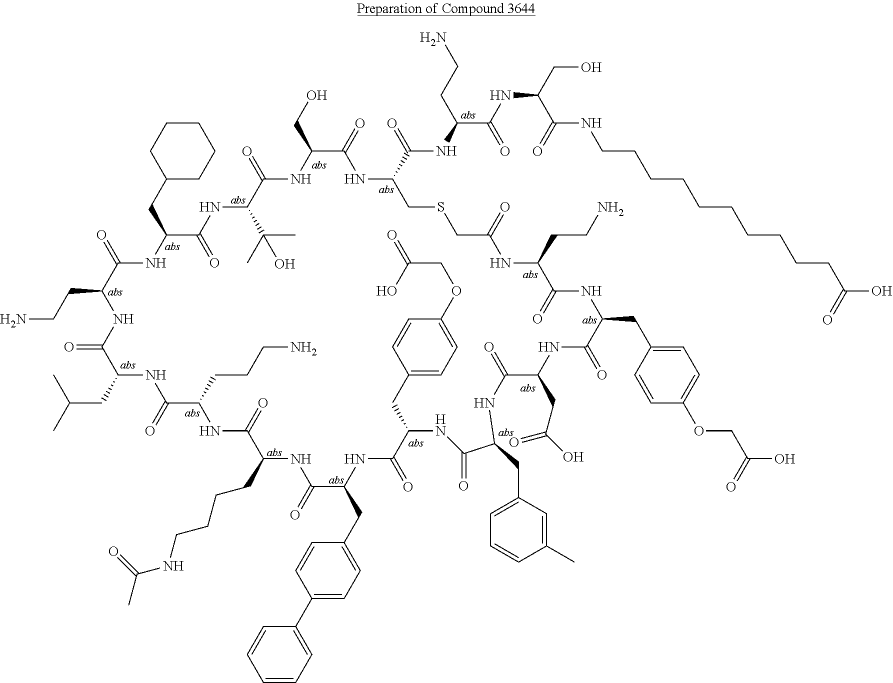 [SMILES: CC(=O)NCCCC[C@@H]1NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)N[C@@H](CO)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC1=O]

Compound 3644 was prepared on a 50 μmol scale. The yield of the product was 9.8 mg, and its estimated purity by LCMS analysis was 95.3%. Analysis condition A: Retention time=1.57 min; ESI-MS(+) m/z [M+2H]²⁺: 1214.1.

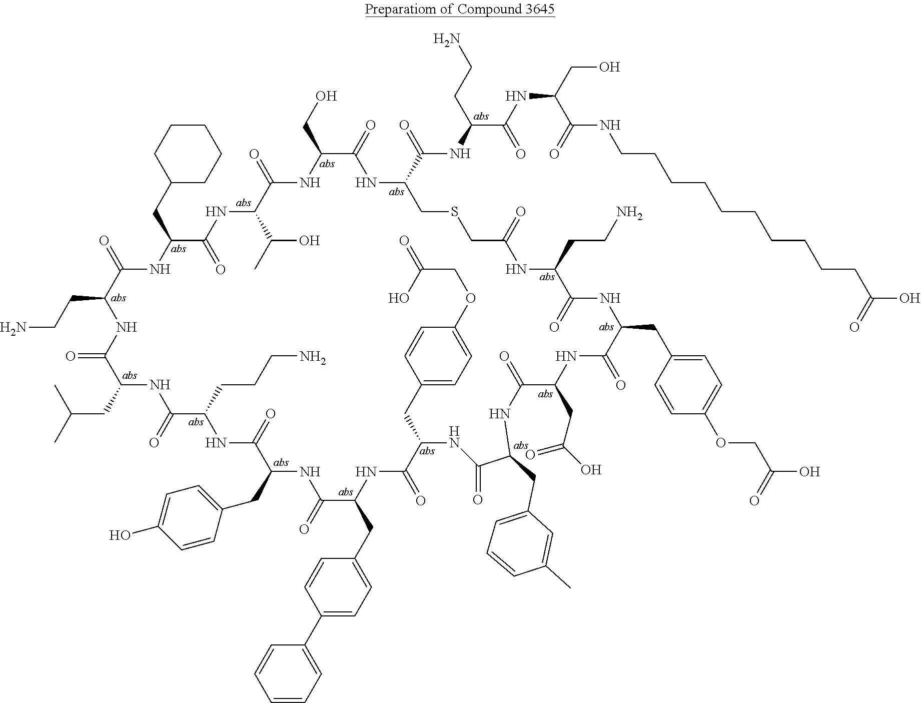 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)N[C@@H](CO)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(OCC(=O)O)cc3)NC2=O)c1]

Compound 3645 was prepared on a 50 μmol scale. The yield of the product was 20.4 mg, and its estimated purity by LCMS analysis was 95.6%. Analysis condition A: Retention time=1.54 min; ESI-MS(+) m/z [M+3H]³⁺: 812.2.

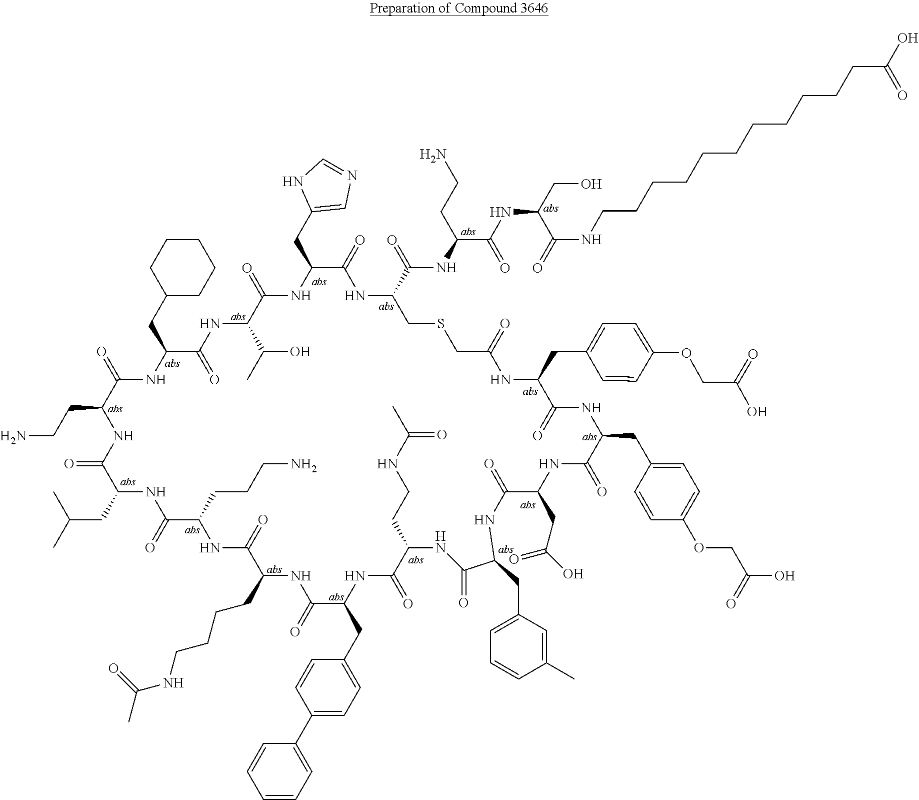 [SMILES: CC(=O)NCCCC[C@@H]1NC(=O)[C@H](Cc2ccc(-c3ccccc3)cc2)NC(=O)[C@H](CCNC(C)=O)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)N[C@@H](CO)C(=O)NCCCCCCCCCCCC(=O)O)NC(=O)[C@H](Cc2cnc[nH]2)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC1=O]

Compound 3646 was prepared on a 50 μmol scale. The yield of the product was 7.1 mg, and its estimated purity by LCMS analysis was 95.4%. Analysis condition A: Retention time=1.51 min; ESI-MS(+) m/z [M+2H]²⁺: 1260.2.

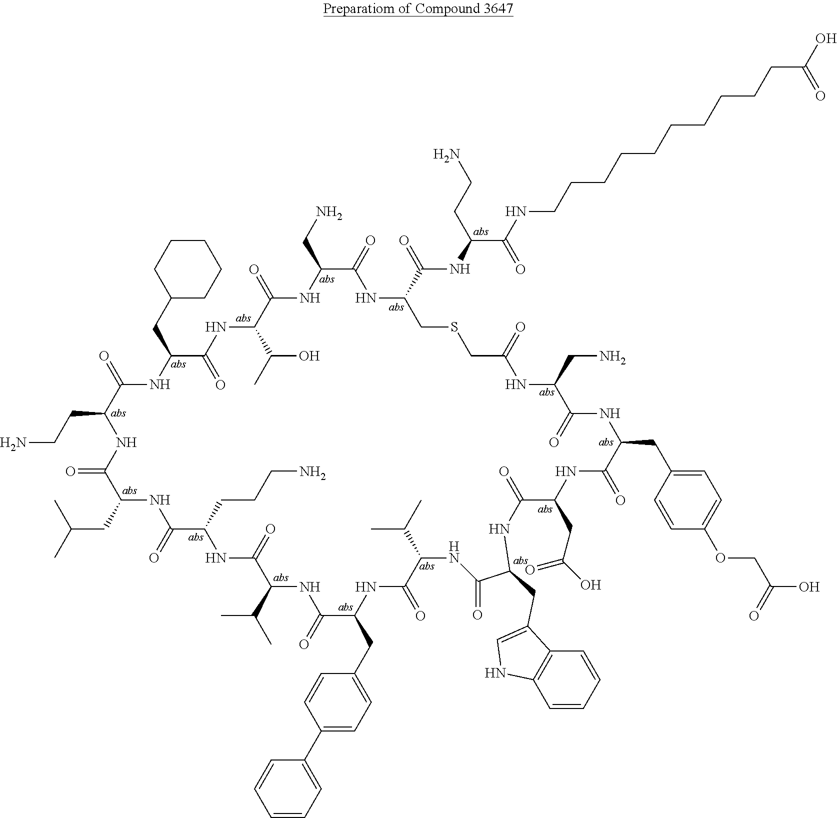 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](CCN)NC1=O]

Compound 3647 was prepared on a 25 μmol scale. The yield of the product was 13.3 mg, and its estimated purity by LCMS analysis was 89.5%. Analysis condition B: Retention time=1.47 min; ESI-MS(+) m/z [M+2H]²⁺: 1072.2.

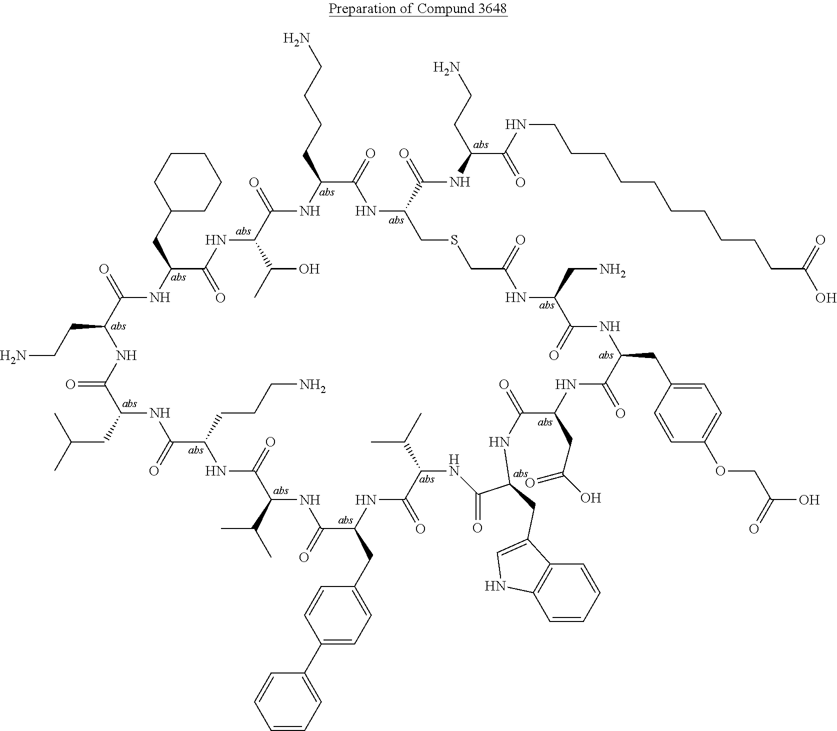 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)O)NC(=O)[C@H](CC2CCCCC2)NC(=O)[C@H](CCN)NC1=O]

Compound 3648 was prepared on a 25 μmol scale. The yield of the product was 9.3 mg, and its estimated purity by LCMS analysis was 98.6%. Analysis condition A: Retention time=1.59 min; ESI-MS(+) m/z [M+3H]³⁺: 729.1.

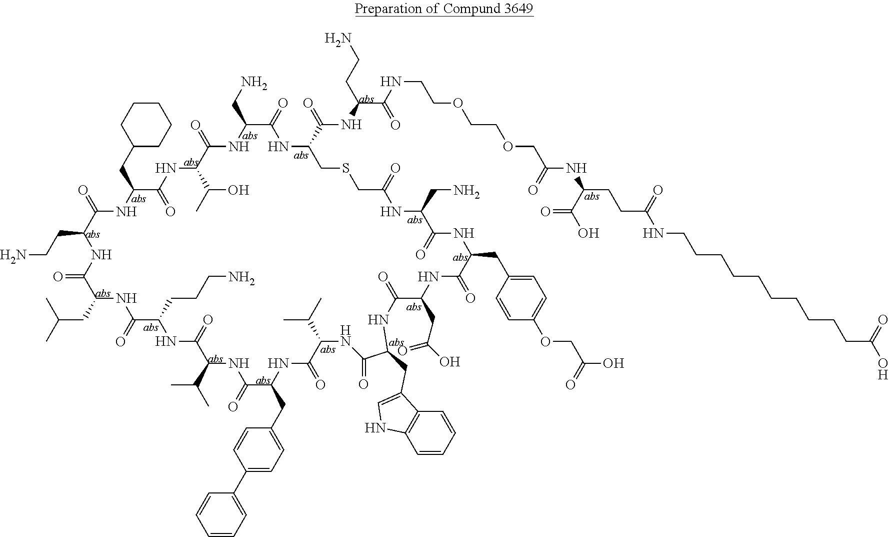 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)N[C@@H](CCC(=O)NCCCCCCCCCCC(=O)O)C(=O)O)NC(=O)[C@H](CN)NC(=O)[C@H](C(C)O)NC(=O)[C@H](CC2CCCCC2)NC(=O)[C@H](CCN)NC1=O]

Compound 3649 was prepared on a 25 μmol scale. The yield of the product was 32.5 mg, and its estimated purity by LCMS analysis was 99.1%. Analysis condition B: Retention time=1.58 min; ESI-MS(+) m/z [M+2H]²⁺: 1208.8.

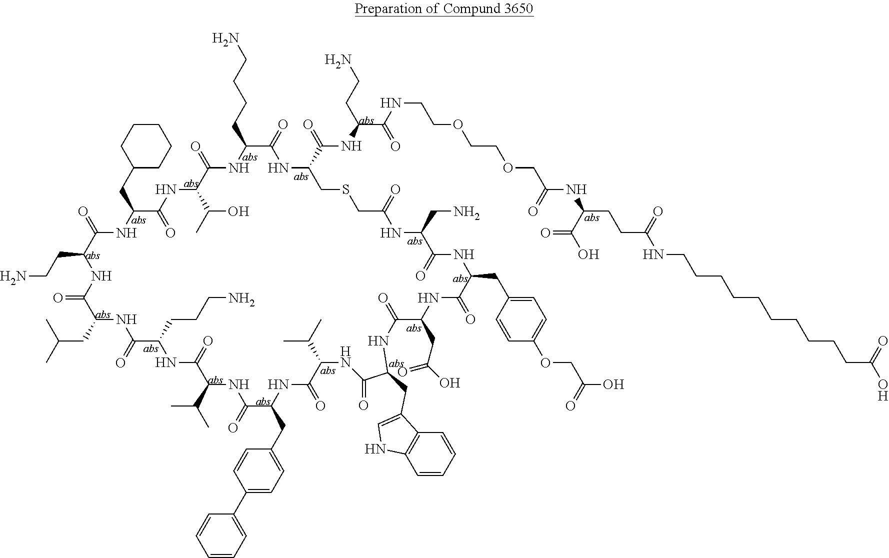 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(=O)N[C@@H](CCC(=O)NCCCCCCCCCCC(=O)O)C(=O)O)NC(=O)[C@H](CCCCN)NC(=O)[C@H](C(C)O)NC(=O)[C@H](CC2CCCCC2)NC(=O)[C@H](CCN)NC1=O]

Compound 3650 was prepared on a 25 μmol scale. The yield of the product was 25.5 mg, and its estimated purity by LCMS analysis was 100%. Analysis condition A: Retention time=1.54 min; ESI-MS(+) m/z [M+2H]²⁺: 1230.4.

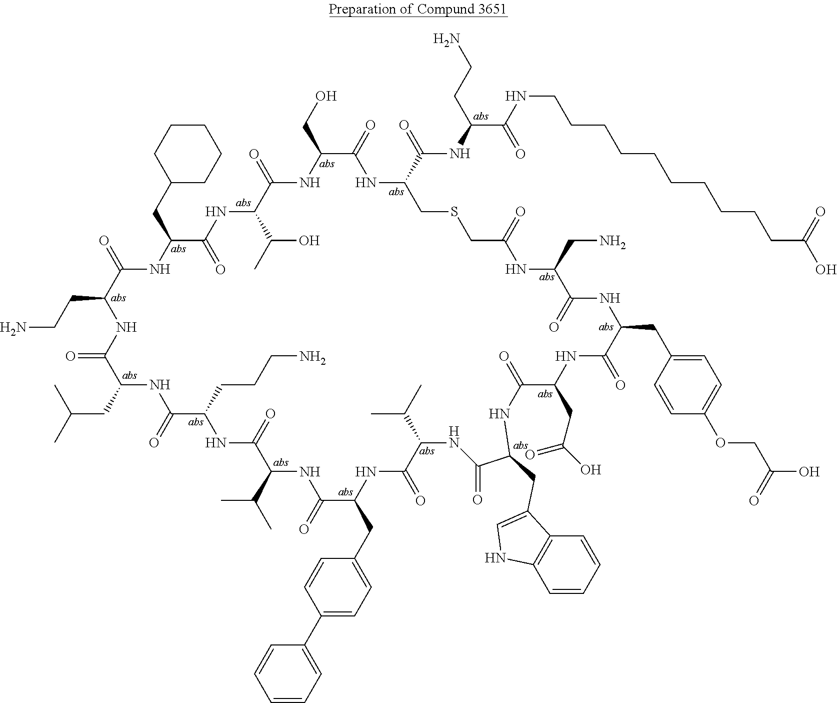 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC1=O]

Compound 3651 was prepared on a 25 μmol scale. The yield of the product was 5.7 mg, and its estimated purity by LCMS analysis was 92.2%. Analysis condition A: Retention time=1.61 min; ESI-MS(+) m/z [M+2H]²⁺: 1072.1.

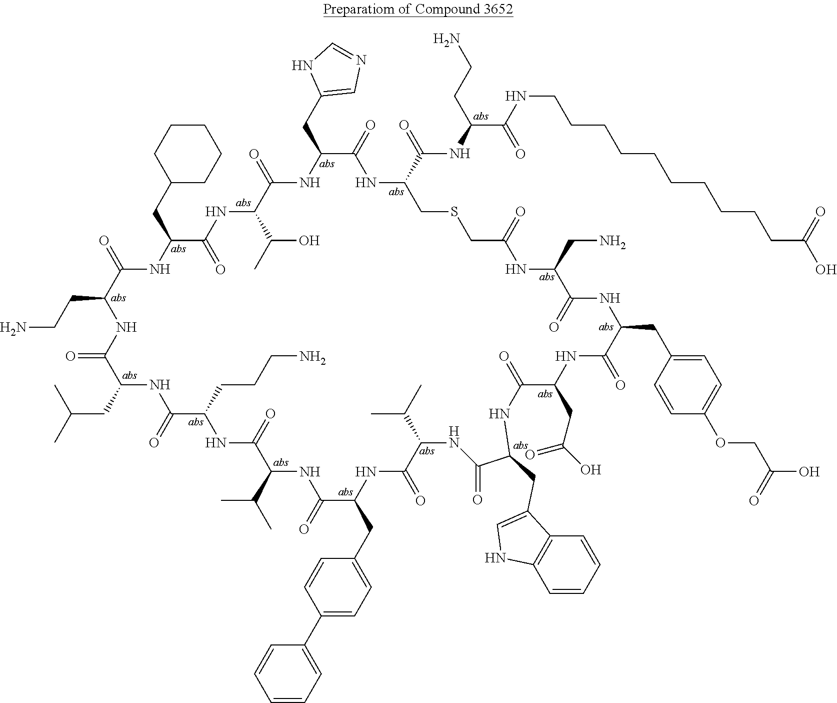 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](Cc2cnc[nH]2)NC(=O)[C@H](C(C)O)NC(=O)[C@H](CC2CCCCC2)NC(=O)[C@H](CCN)NC1=O]

Compound 3652 was prepared on a 25 μmol scale. The yield of the product was 20.5 mg, and its estimated purity by LCMS analysis was 95.2%. Analysis condition A: Retention time=1.71 min; ESI-MS(+) m/z [M+3H]³⁺: 732.2.

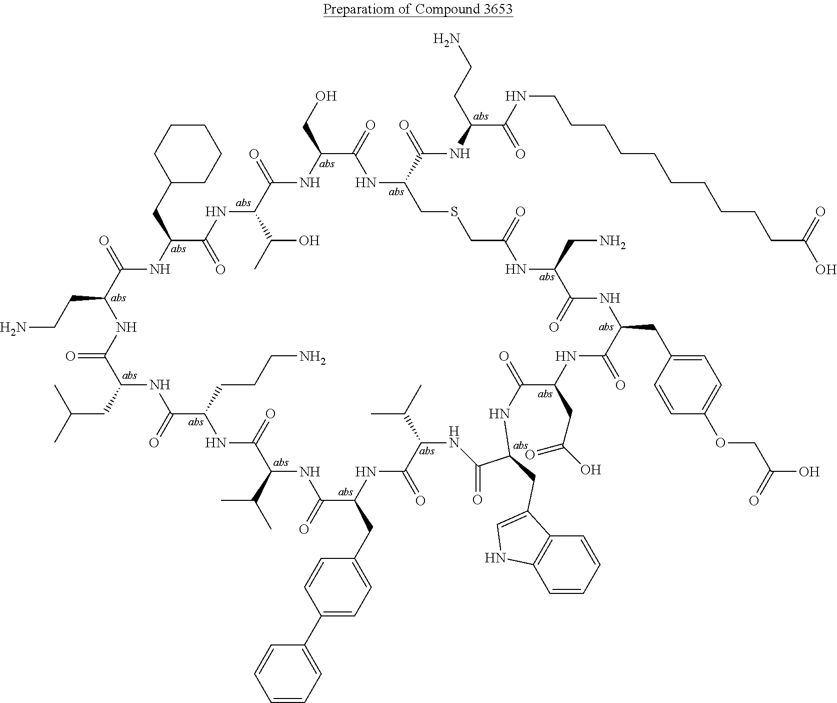 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC1=O]

Compound 3653 was prepared on a 25 μmol scale. The yield of the product was 4 mg, and its estimated purity by LCMS analysis was 92.7%. Analysis condition B: Retention time=1.47 min; ESI-MS(+) m/z [M+2H]²⁺: 1086.1.

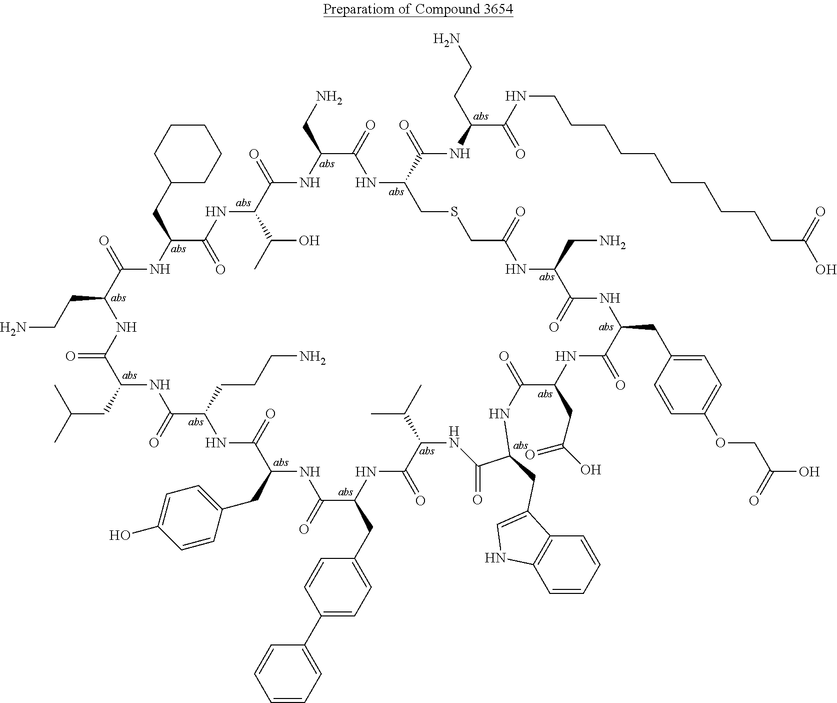 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](CCN)NC1=O]

Compound 3654 was prepared on a 25 μmol scale. The yield of the product was 25.1 mg, and its estimated purity by LCMS analysis was 95.6%. Analysis condition A: Retention time=1.6 min; ESI-MS(+) m/z [M+3H]³⁺: 736.2.

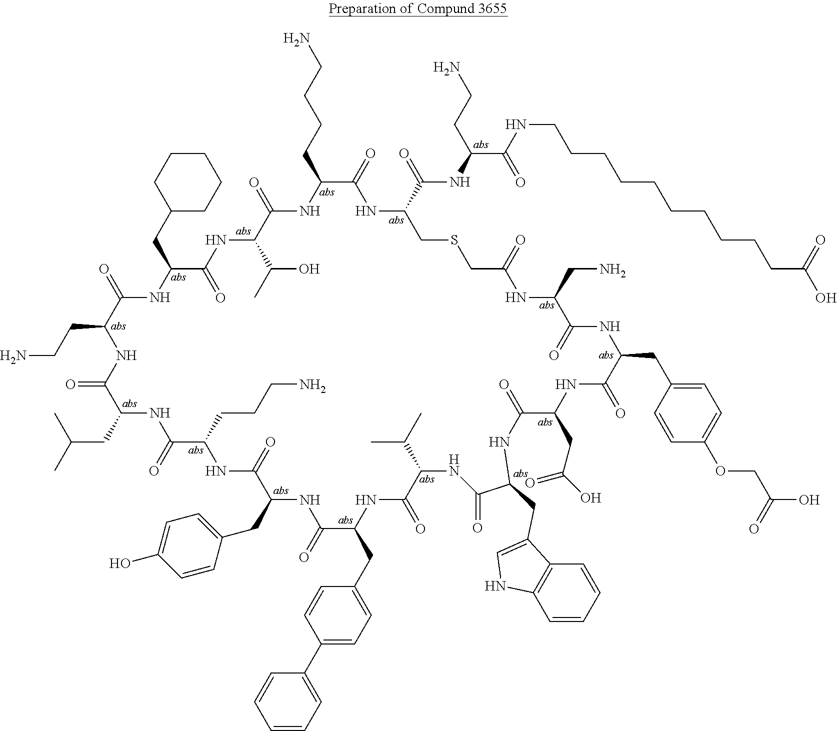 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)O)NC(=O)[C@H](CC2CCCCC2)NC(=O)[C@H](CCN)NC1=O]

Compound 3655 was prepared on a 25 μmol scale. The yield of the product was 12.6 mg, and its estimated purity by LCMS analysis was 95.4%. Analysis condition B: Retention time=1.45 min; ESI-MS(+) m/z [M+2H]²⁺: 1125.1.

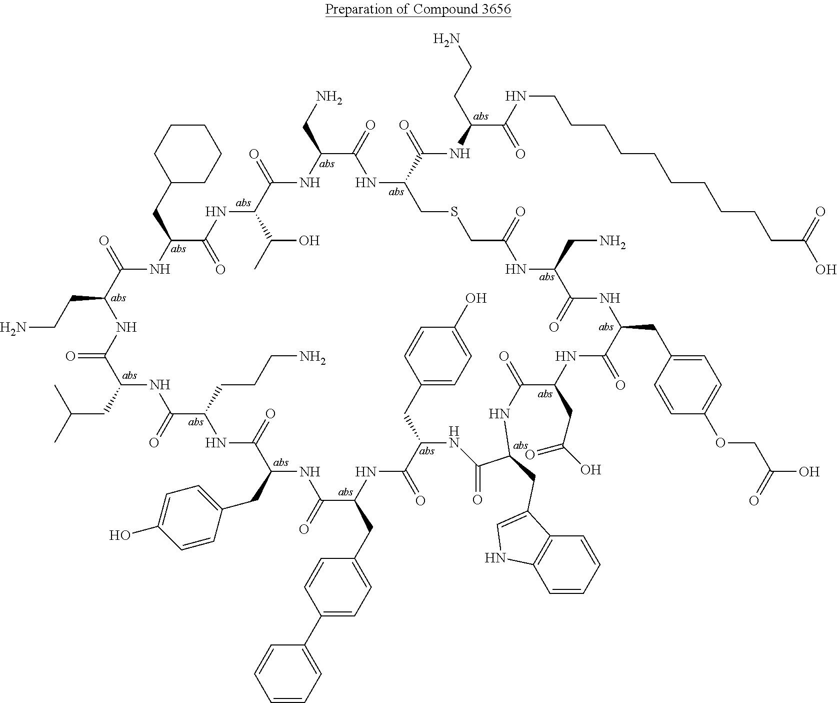 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](CCN)NC1=O]

Compound 3656 was prepared on a 25 μmol scale. The yield of the product was 6.9 mg, and its estimated purity by LCMS analysis was 96.3%. Analysis condition A: Retention time=1.6 min; ESI-MS(+) m/z [M+2H]²⁺: 1136.1.

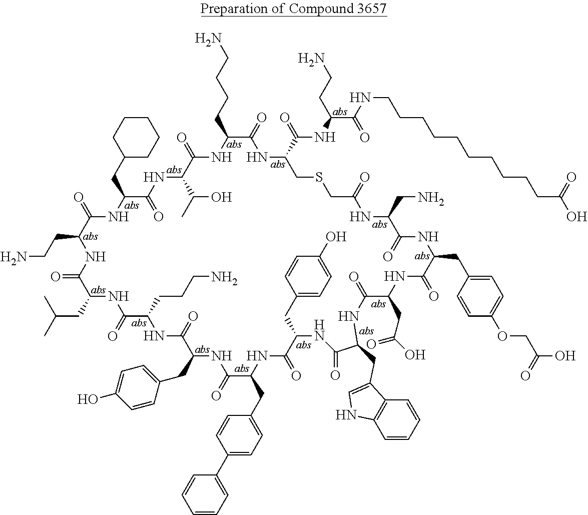 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)O)NC(=O)[C@H](CC2CCCCC2)NC(=O)[C@H](CCN)NC1=O]

Compound 3657 was prepared on a 25 μmol scale. The yield of the product was 13.1 mg, and its estimated purity by LCMS analysis was 100%. Analysis condition A: Retention time=1.51 min; ESI-MS(+) m/z [M+2H]²⁺: 1157.3.

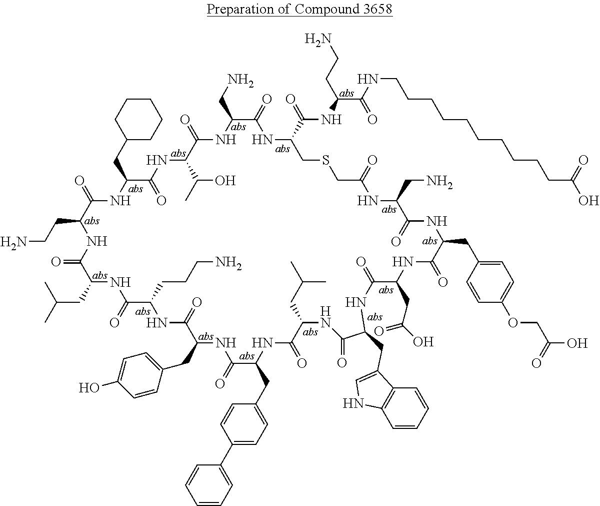 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 3658 was prepared on a 25 μmol scale. The yield of the product was 38.9 mg, and its estimated purity by LCMS analysis was 95.7%. Analysis condition A: Retention time=1.64 min; ESI-MS(+) m/z [M+2H]²⁺: 1111.3.

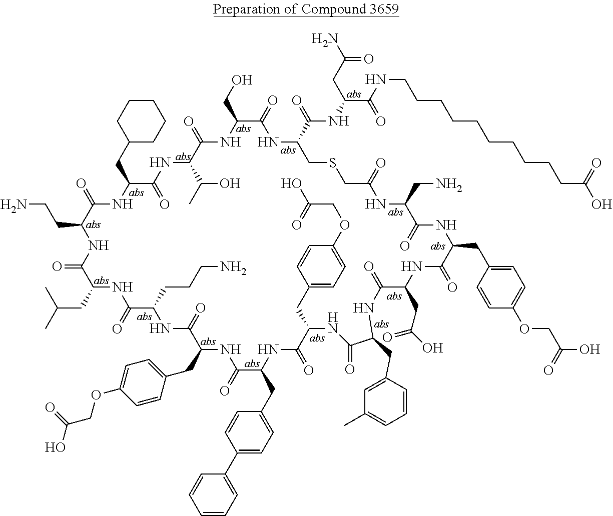 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@H](CC(N)=O)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3659 was prepared on a 25 μmol scale. The yield of the product was 6.9 mg, and its estimated purity by LCMS analysis was 97.9%. Analysis condition B: Retention time=1.73 min; ESI-MS(+) m/z [M+2H]²⁺: 1189.

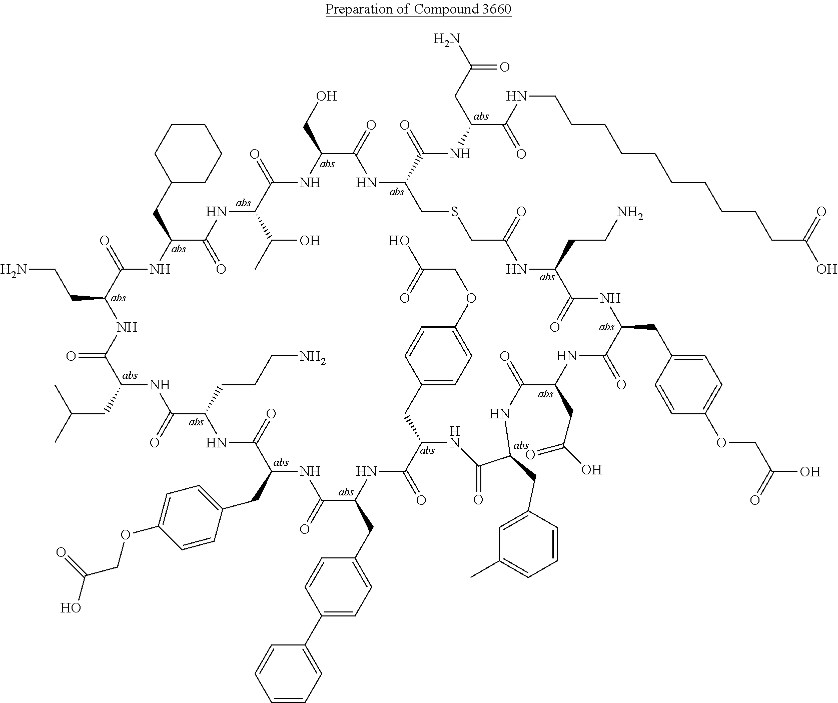 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@H](CC(N)=O)C(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3660 was prepared on a 25 μmol scale. The yield of the product was 15.8 mg, and its estimated purity by LCMS analysis was 95.5%. Analysis condition B: Retention time=1.58 min; ESI-MS(+) m/z [M+2H]²⁺: 1196.

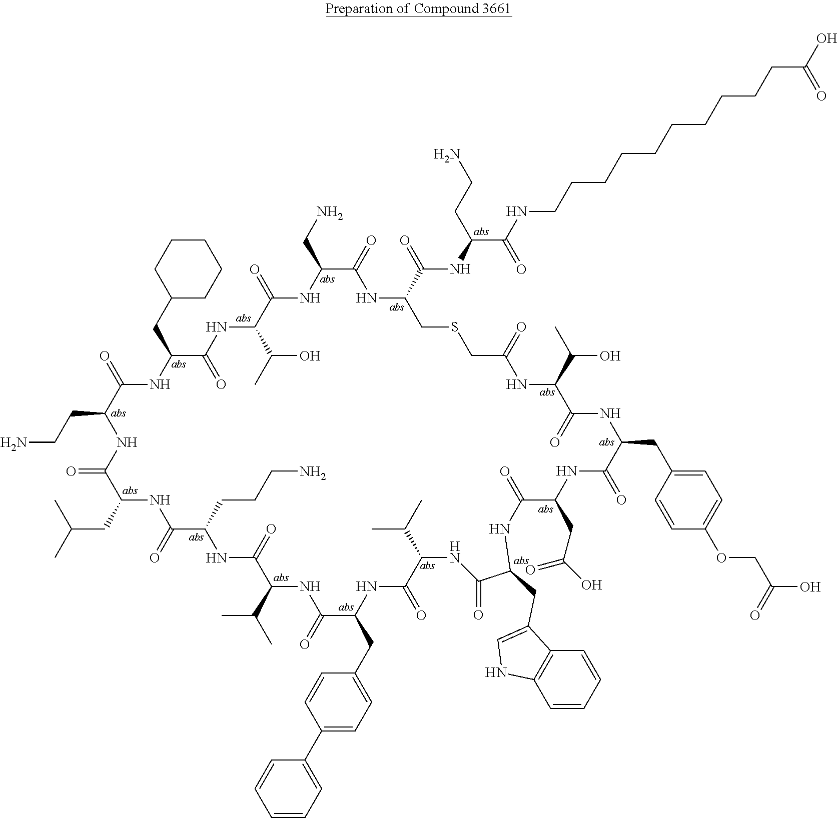 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](CCN)NC1=O]

Compound 3661 was prepared on a 25 μmol scale. The yield of the product was 25.6 mg, and its estimated purity by LCMS analysis was 99%. Analysis condition A: Retention time=1.74 min; ESI-MS(+) m/z [M+3H]³⁺: 720.

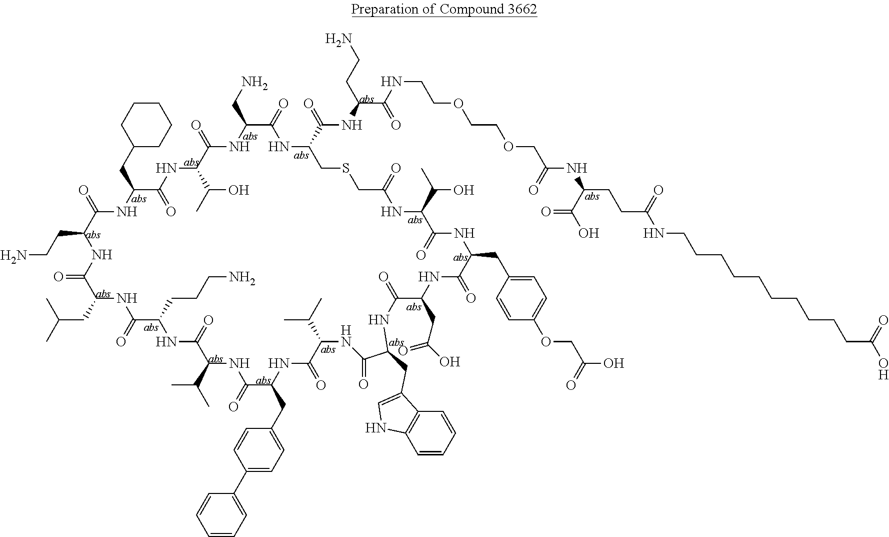 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)N[C@@H](CCC(=O)NCCCCCCCCCCC(=O)O)C(=O)O)NC(=O)[C@H](CN)NC(=O)[C@H](C(C)O)NC(=O)[C@H](CC2CCCCC2)NC(=O)[C@H](CCN)NC1=O]

Compound 3662 was prepared on a 25 μmol scale. The yield of the product was 13.1 mg, and its estimated purity by LCMS analysis was 100%. Analysis condition A: Retention time=1.61 min; ESI-MS(+) m/z [M+3H]³⁺: 811.5.

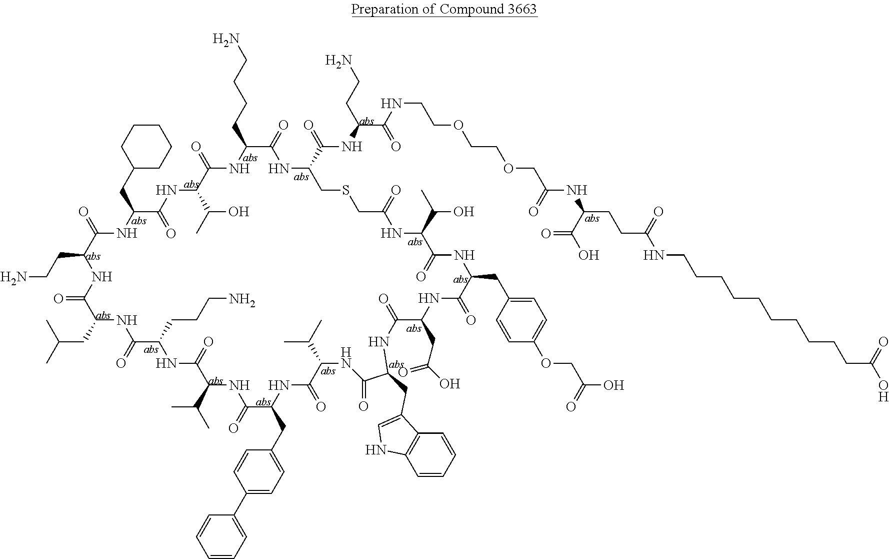 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)N[C@@H](CCC(=O)NCCCCCCCCCCC(=O)O)C(=O)O)NC(=O)[C@H](CCCCN)NC(=O)[C@H](C(C)O)NC(=O)[C@H](CC2CCCCC2)NC(=O)[C@H](CCN)NC1=O]

Compound 3663 was prepared on a 25 μmol scale. The yield of the product was 26 mg, and its estimated purity by LCMS analysis was 95.5%. Analysis condition B: Retention time=1.55 min; ESI-MS(+) m/z [M+3H]³⁺: 825.1.

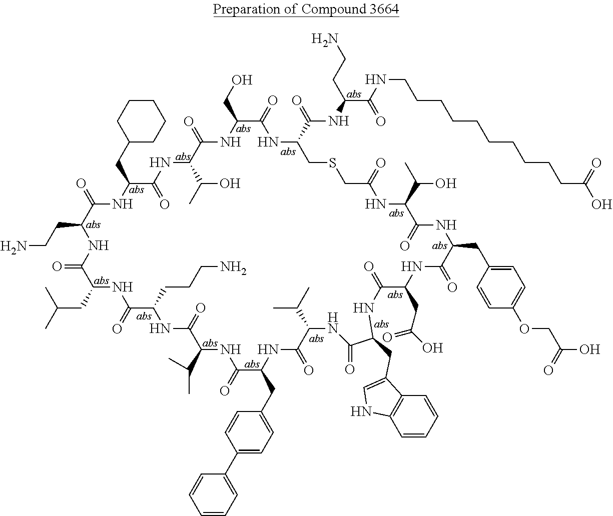 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC1=O]

Compound 3664 was prepared on a 25 μmol scale. The yield of the product was 2.1 mg, and its estimated purity by LCMS analysis was 94.8%. Analysis condition B: Retention time=1.71 min; ESI-MS(+) m/z [M+2H]²⁺: 1079.9.

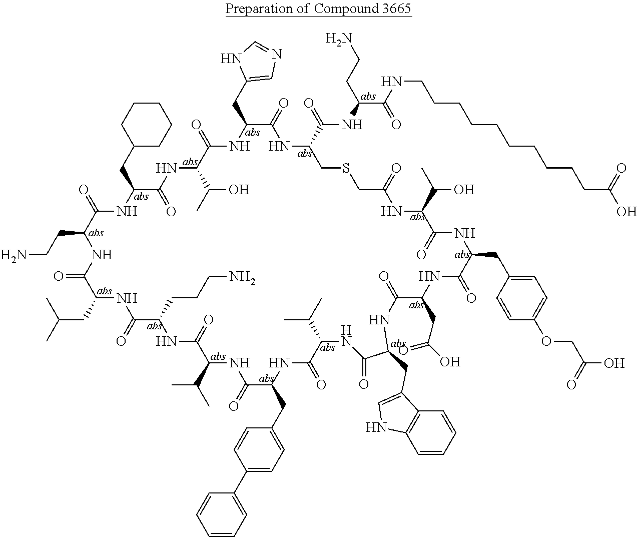 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](Cc2cnc[nH]2)NC(=O)[C@H](C(C)O)NC(=O)[C@H](CC2CCCCC2)NC(=O)[C@H](CCN)NC1=O]

Compound 3665 was prepared on a 25 μmol scale. The yield of the product was 4.1 mg, and its estimated purity by LCMS analysis was 98.9%. Analysis condition A: Retention time=1.68 min; ESI-MS(+) m/z [M+3H]³⁺: 737.1.

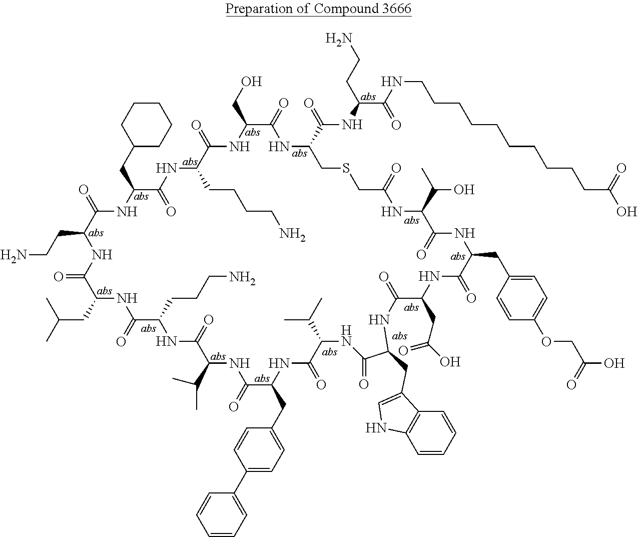 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](CCCCN)NC(=O)[C@H](CC2CCCCC2)NC(=O)[C@H](CCN)NC1=O]

Compound 3666 was prepared on a 25 μmol scale. The yield of the product was 3.7 mg, and its estimated purity by LCMS analysis was 98.6%. Analysis condition B: Retention time=1.59 min; ESI-MS(+) m/z [M+2H]²⁺: 1093.2.

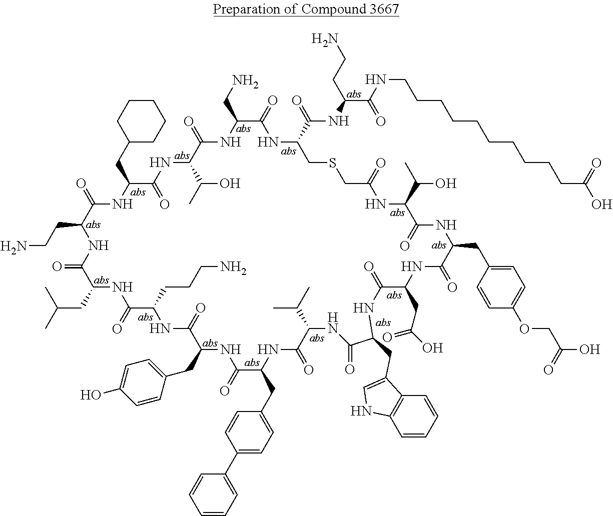 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](CCN)NC1=O]

Compound 3667 was prepared on a 25 μmol scale. The yield of the product was 8.5 mg, and its estimated purity by LCMS analysis was 95.5%. Analysis condition A: Retention time-1.6 min; ESI-MS(+) m/z [M+3H]³⁺: 741.3.

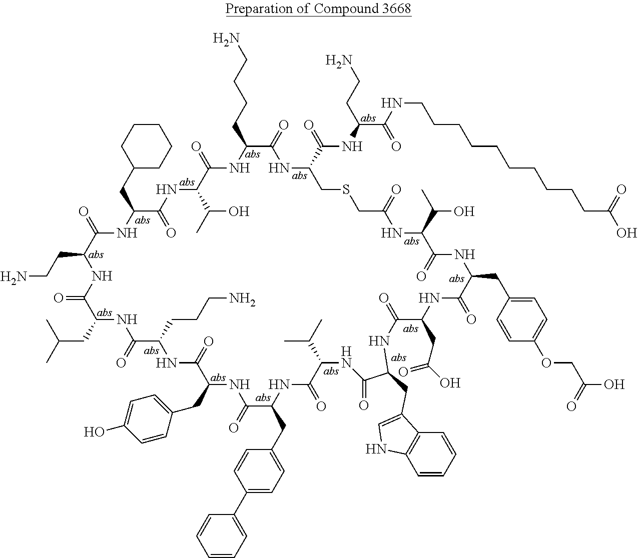 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCCN)NC(=O)[C@H](C(C)O)NC(=O)[C@H](CC2CCCCC2)NC(=O)[C@H](CCN)NC1=O]

Compound 3668 was prepared on a 25 μmol scale. The yield of the product was 2 mg, and its estimated purity by LCMS analysis was 83%. Analysis condition B: Retention time=1.58 min; ESI-MS(+) m/z [M+2H]²⁺: 1132.4.

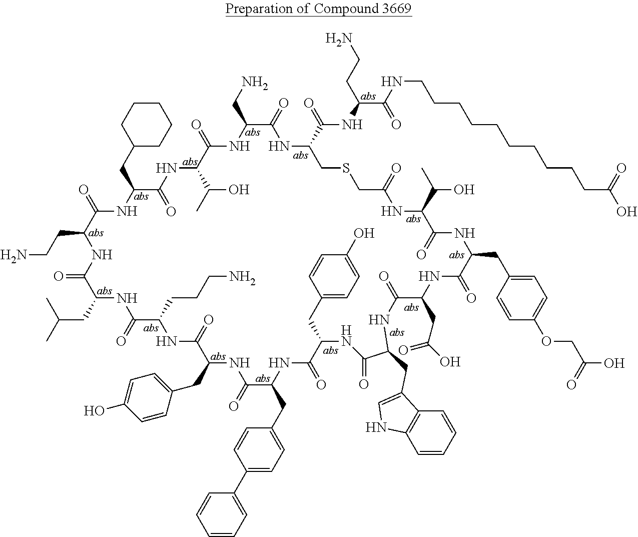 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](CCN)NC1=O]

Compound 3669 was prepared on a 25 μmol scale. The yield of the product was 8.6 mg, and its estimated purity by LCMS analysis was 95.8%. Analysis condition B: Retention time=1.6 min; ESI-MS(+) m/z [M+2H]²⁺: 1143.

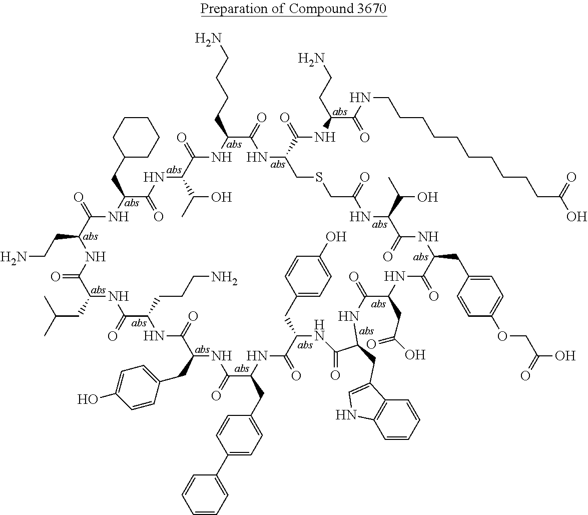 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCCN)NC(=O)[C@H](C(C)O)NC(=O)[C@H](CC2CCCCC2)NC(=O)[C@H](CCN)NC1=O]

Compound 3670 was prepared on a 25 μmol scale. The yield of the product was 9.6 mg, and its estimated purity by LCMS analysis was 90%. Analysis condition B: Retention time=1.51 min; ESI-MS(+) m/z [M+3H]³⁺: 777.1.

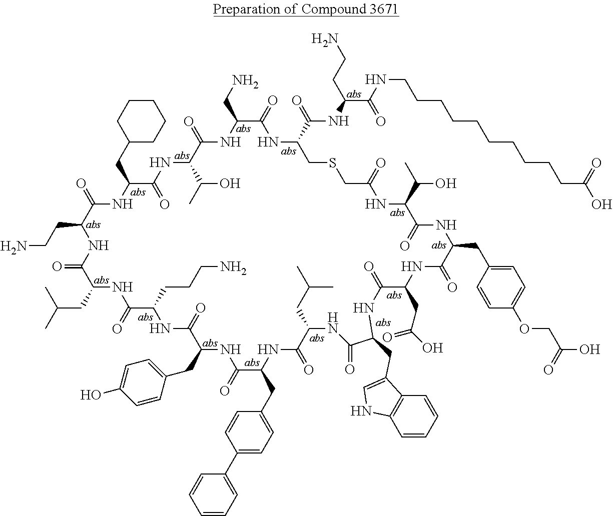 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 3671 was prepared on a 25 μmol scale. The yield of the product was 15.1 mg, and its estimated purity by LCMS analysis was 98.5%. Analysis condition A: Retention time=1.65 min; ESI-MS(+) m/z [M+3H]³⁺: 746.2.

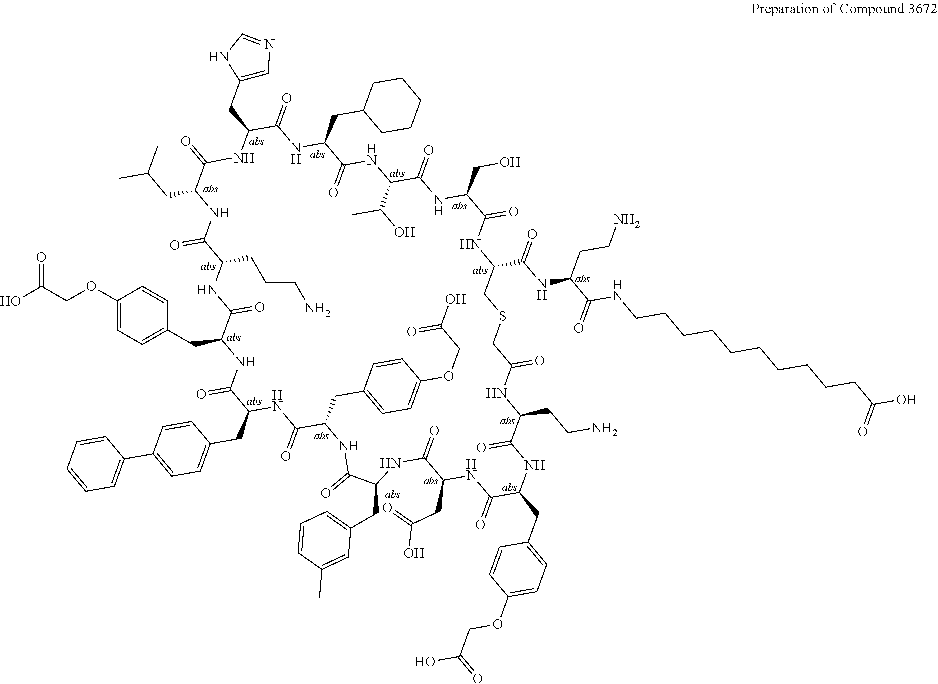 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3672 was prepared on a 50 μmol scale. The yield of the product was 15.3 mg, and its estimated purity by LCMS analysis was 97.1%. Analysis condition A: Retention time=1.55 min; ESI-MS(+) m/z [M+2H]²⁺: 1207.2.

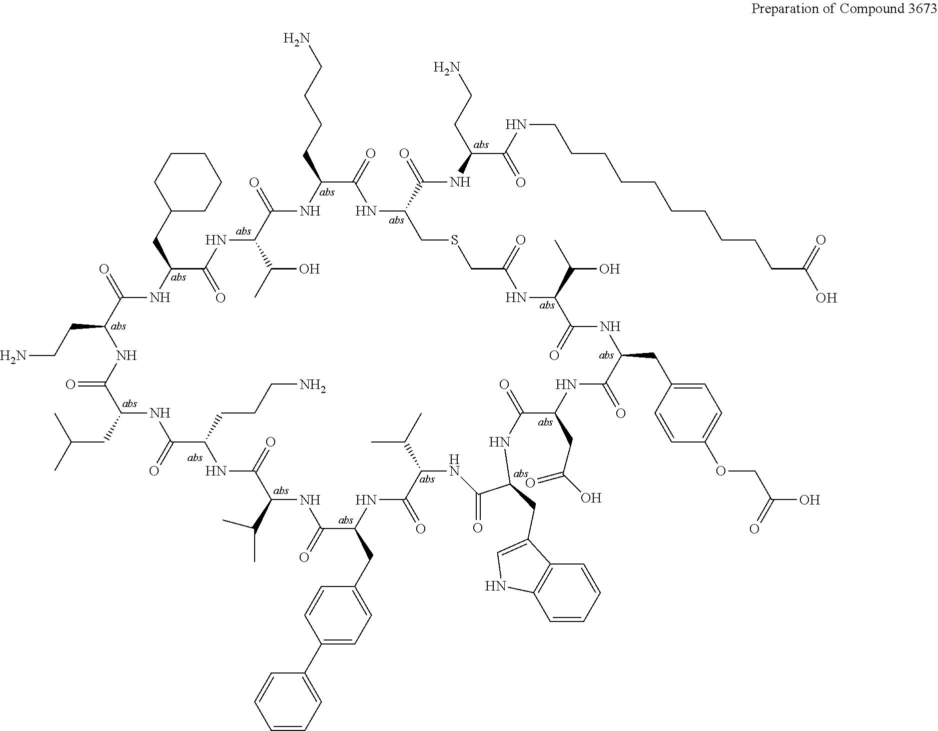 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCCN)NC(=O)[C@H](C(C)O)NC(=O)[C@H](CC2CCCCC2)NC(=O)[C@H](CCN)NC1=O]

Compound 3673 was prepared on a 25 μmol scale. The yield of the product was 13.2 mg, and its estimated purity by LCMS analysis was 90.8%. Analysis condition B: Retention time=1.47 min; ESI-MS(+) m/z [M+2H]²⁺: 1100.1.

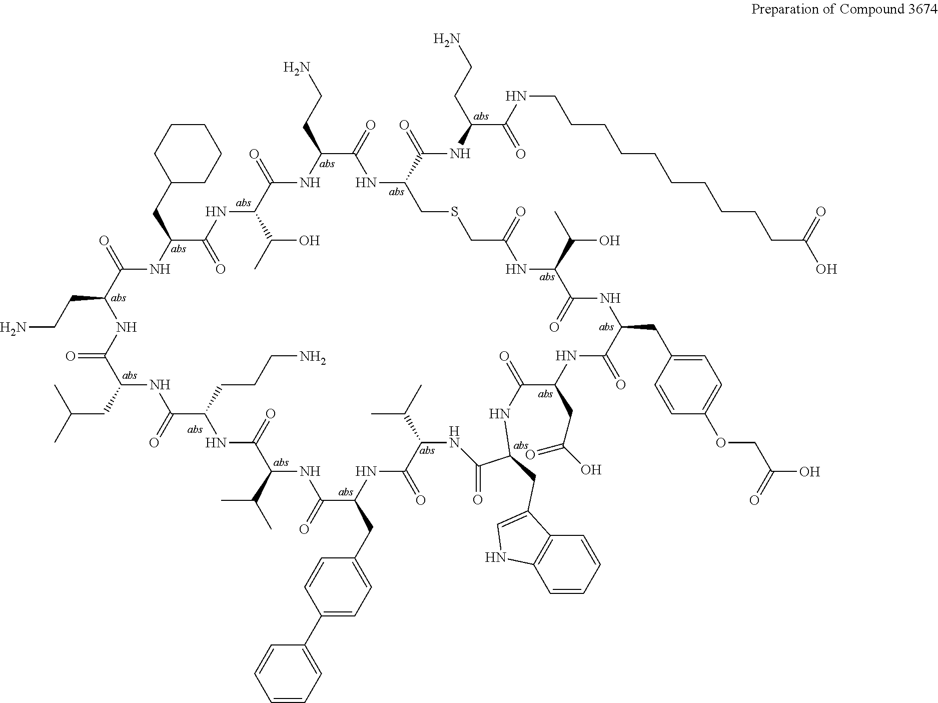 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCN)NC(=O)[C@H](C(C)O)NC(=O)[C@H](CC2CCCCC2)NC(=O)[C@H](CCN)NC1=O]

Compound 3674 was prepared on a 50 μmol scale. The yield of the product was 25.9 mg, and its estimated purity by LCMS analysis was 99.2%. Analysis condition A: Retention time=1.66 min; ESI-MS(+) m/z [M+2H]²⁺: 1086.1.

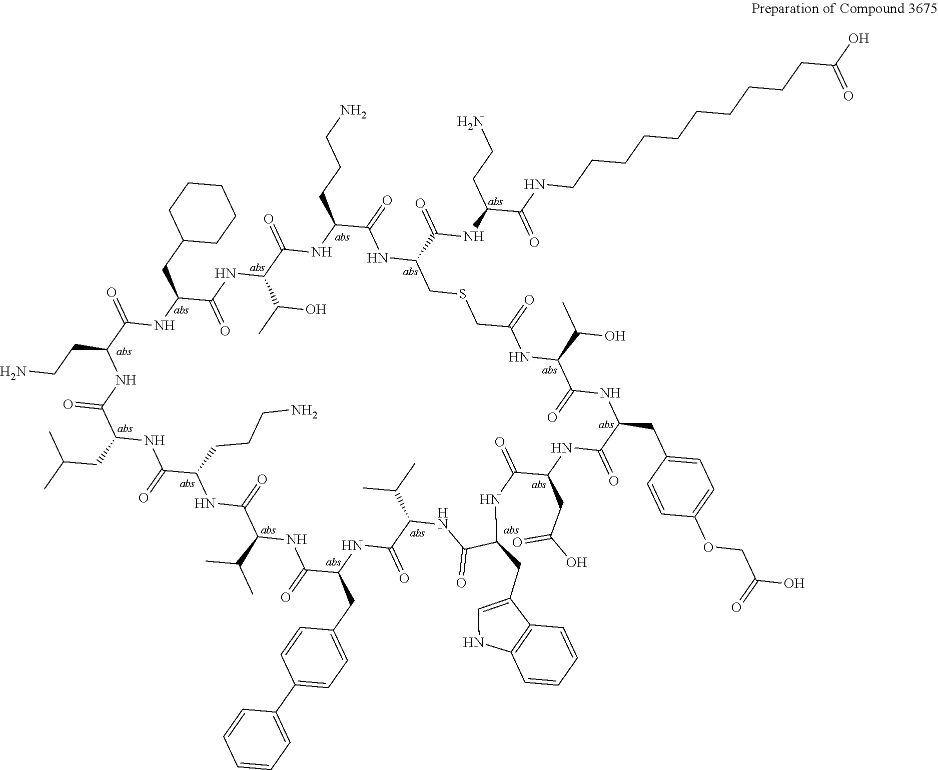 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCN)NC(=O)[C@H](C(C)O)NC(=O)[C@H](CC2CCCCC2)NC(=O)[C@H](CCN)NC1=O]

Compound 3675 was prepared on a 50 μmol scale. The yield of the product was 19.3 mg, and its estimated purity by LCMS analysis was 95.6%. Analysis condition B: Retention time=1.6 min; ESI-MS(+) m/z [M+3H]³⁺: 729.2.

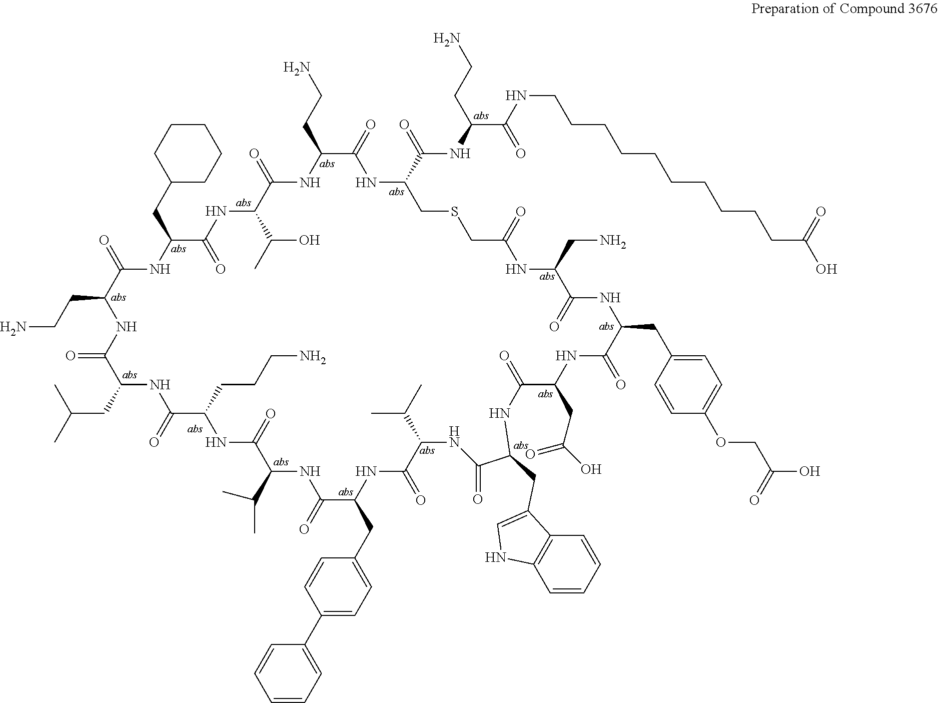 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCN)NC(=O)[C@H](C(C)O)NC(=O)[C@H](CC2CCCCC2)NC(=O)[C@H](CCN)NC1=O]

Compound 3676 was prepared on a 50 μmol scale. The yield of the product was 27.7 mg, and its estimated purity by LCMS analysis was 97.4%. Analysis condition A: Retention time=1.61 min; ESI-MS(+) m/z [M+2H]²⁺: 1079.2.

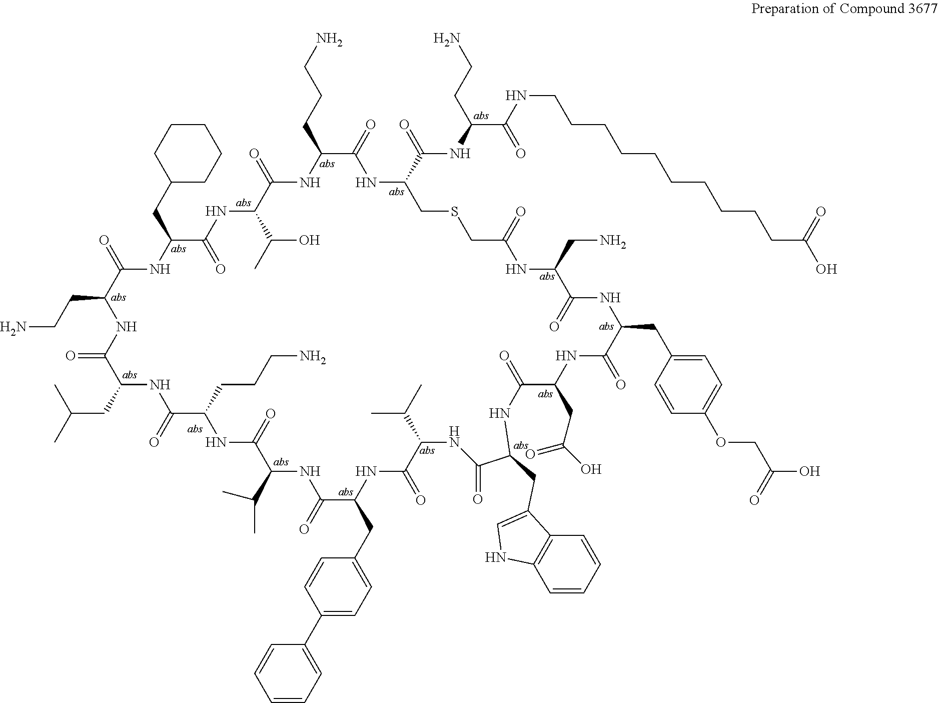 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)O)NC(=O)[C@H](CC2CCCCC2)NC(=O)[C@H](CCN)NC1=O]

Compound 3677 was prepared on a 50 μmol scale. The yield of the product was 26.7 mg, and its estimated purity by LCMS analysis was 98.5%. Analysis condition B: Retention time=1.5 min; ESI-MS(+) m/z [M+3H]³⁺: 724.2.

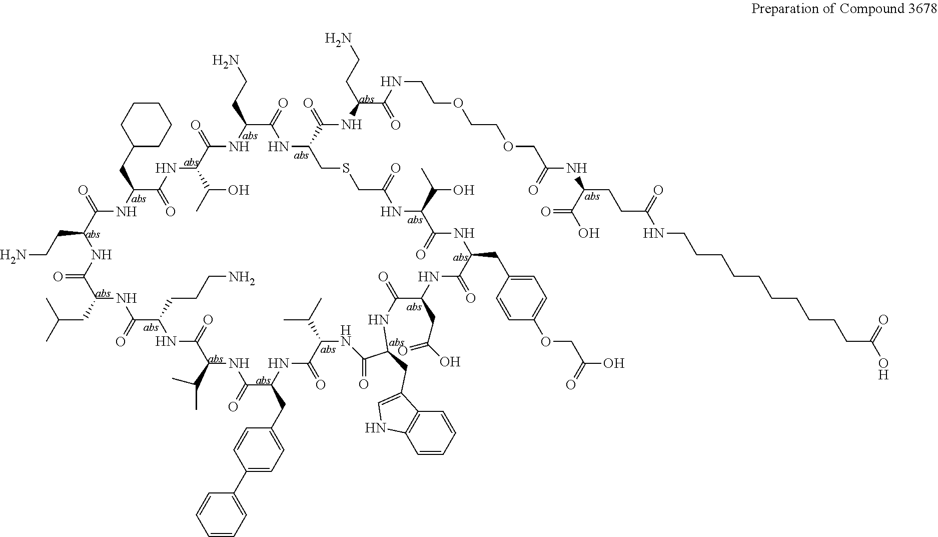 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)N[C@@H](CCC(=O)NCCCCCCCCCCC(=O)O)C(=O)O)NC(=O)[C@H](CCN)NC(=O)[C@H](C(C)O)NC(=O)[C@H](CC2CCCCC2)NC(=O)[C@H](CCN)NC1=O]

Compound 3678 was prepared on a 50 μmol scale. The yield of the product was 35.8 mg, and its estimated purity by LCMS analysis was 95.3%. Analysis condition A: Retention time=1.65 min; ESI-MS(+) m/z [M+3H]³⁺: 816.2.

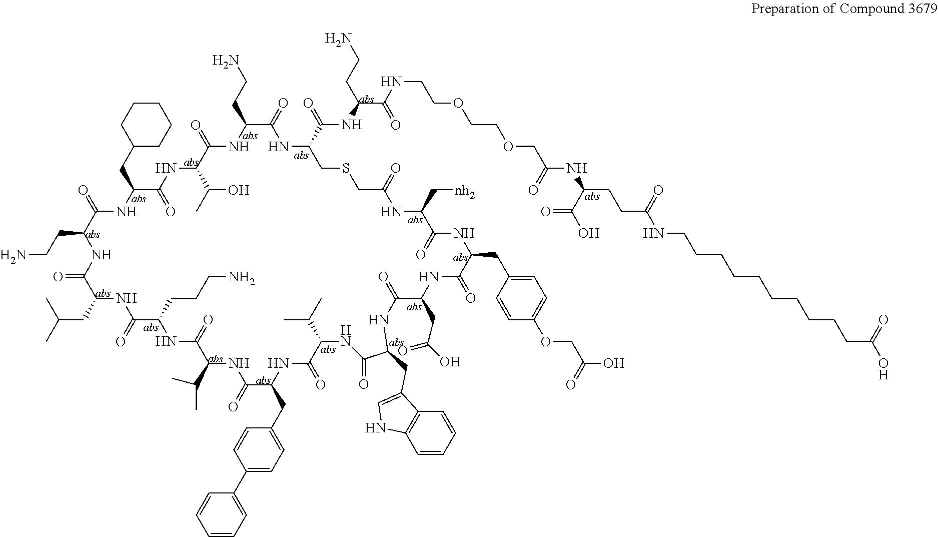

Compound 3679 was prepared on a 50 μmol scale. The yield of the product was 45.4 mg, and its estimated purity by LCMS analysis was 96%. Analysis condition A: Retention time=1.61, 1.65 min; ESI-MS(+) m/z [M+3H]³⁺: 811.08, 811.08.

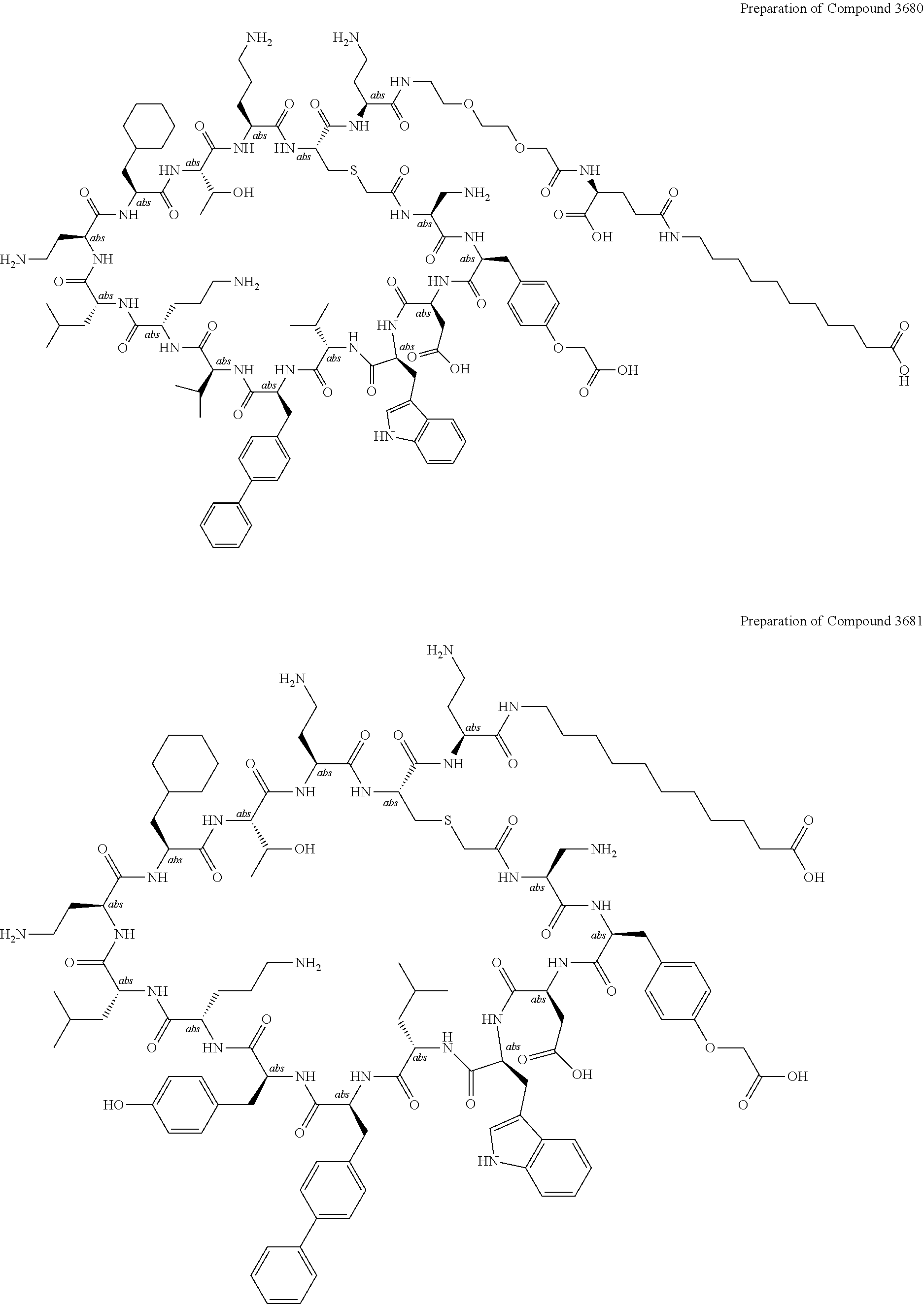

Compound 3680 was prepared on a 50 μmol scale. The yield of the product was 10.5 mg, and its estimated purity by LCMS analysis was 95.1%. Analysis condition B: Retention time=1.55 min; ESI-MS(+) m/z [M+3H]³⁺: 816.

Compound 3681 was prepared on a 50 μmol scale. The yield of the product was 29.2 mg, and its estimated purity by LCMS analysis was 95.3%. Analysis condition B: Retention time=1.59 min; ESI-MS(+) m/z [M+2H]²⁺: 1117.7.

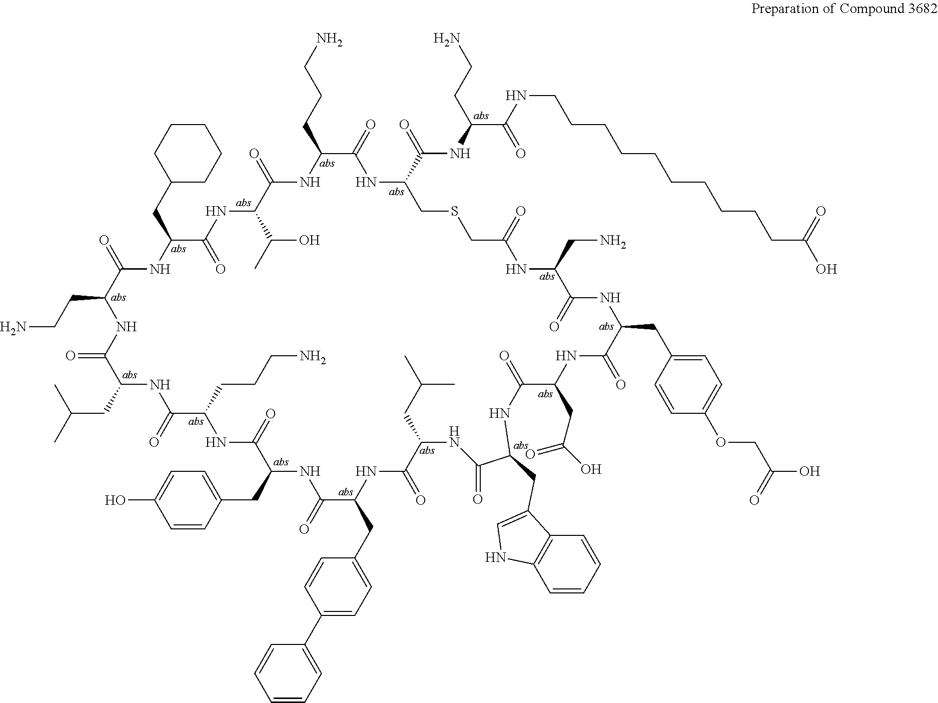 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 3682 was prepared on a 50 μmol scale. The yield of the product was 62.6 mg, and its estimated purity by LCMS analysis was 97.9%. Analysis condition A: Retention time=1.62 min; ESI-MS(+) m/z [M+2H]²⁺: 1125.

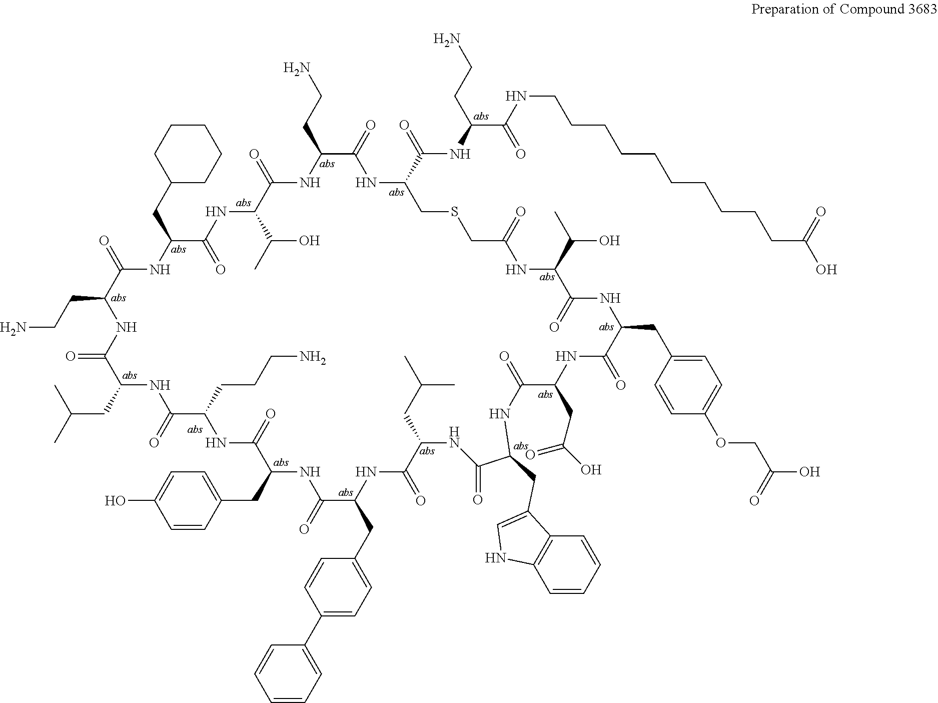 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 3683 was prepared on a 50 μmol scale. The yield of the product was 37.7 mg, and its estimated purity by LCMS analysis was 98.6%. Analysis condition A: Retention time=1.64 min; ESI-MS(+) m/z [M+3H]³⁺: 751.

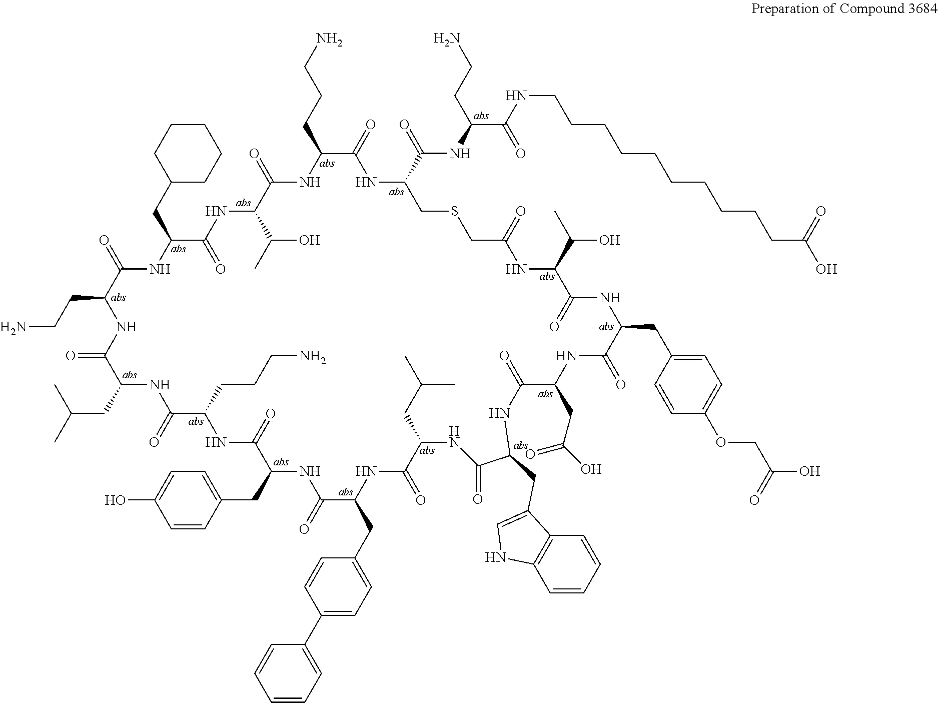 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 3684 was prepared on a 50 μmol scale. The yield of the product was 30.8 mg, and its estimated purity by LCMS analysis was 97.3%. Analysis condition B: Retention time=1.63 min; ESI-MS(+) m/z [M+3H]³⁺: 755.

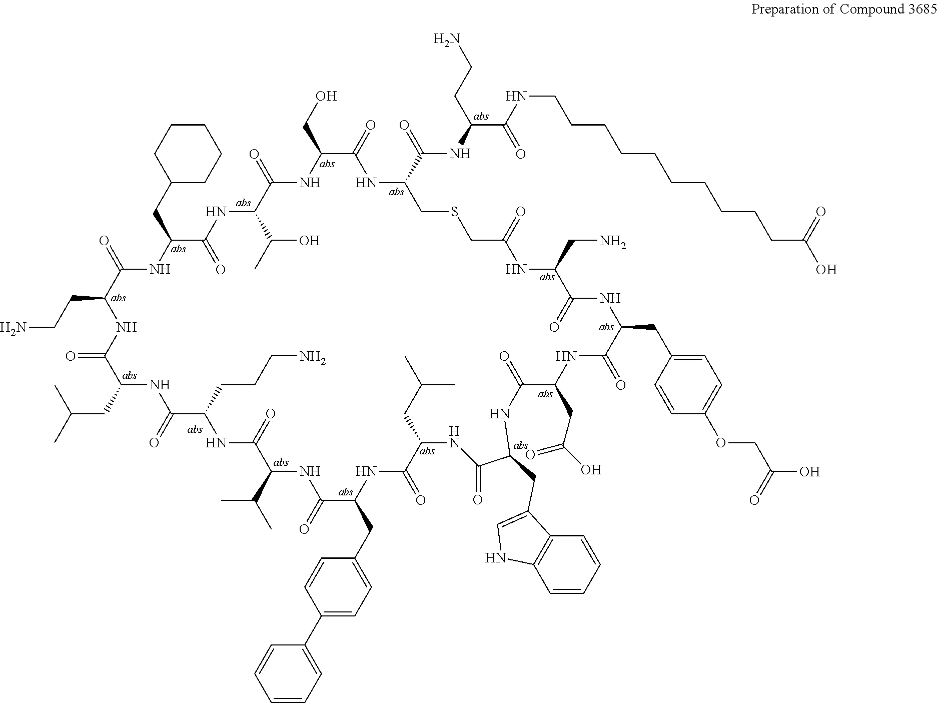 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3685 was prepared on a 50 μmol scale. The yield of the product was 9.2 mg, and its estimated purity by LCMS analysis was 95.8%. Analysis condition B: Retention time=1.5 min; ESI-MS(+) m/z [M+3H]³⁺: 720.1.

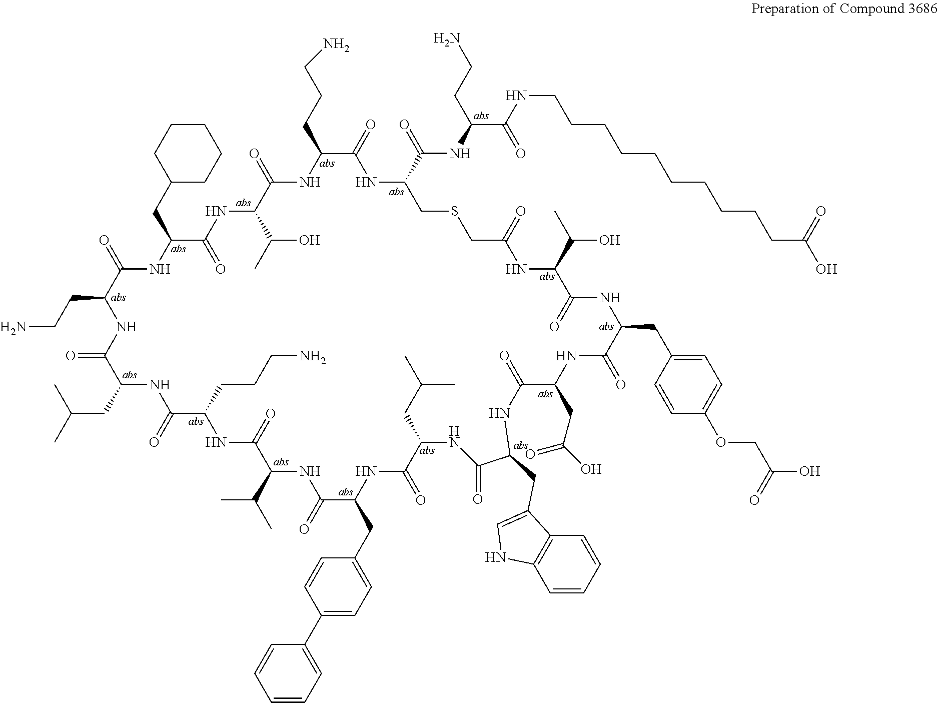 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3686 was prepared on a 50 μmol scale. The yield of the product was 14.2 mg, and its estimated purity by LCMS analysis was 95.4%. Analysis condition A: Retention time=1.7 min; ESI-MS(+) m/z [M+3H]³⁺: 734.2.

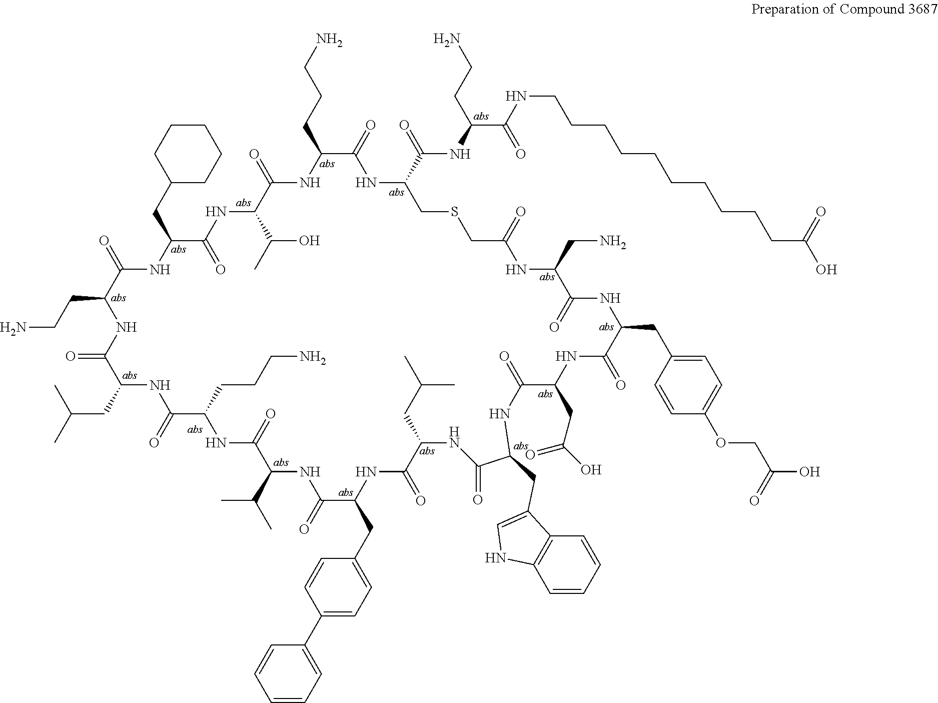 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3687 was prepared on a 50 μmol scale. The yield of the product was 26 mg, and its estimated purity by LCMS analysis was 98.7%. Analysis condition A: Retention time=1.63 min; ESI-MS(+) m/z [M+3H]³⁺: 729.

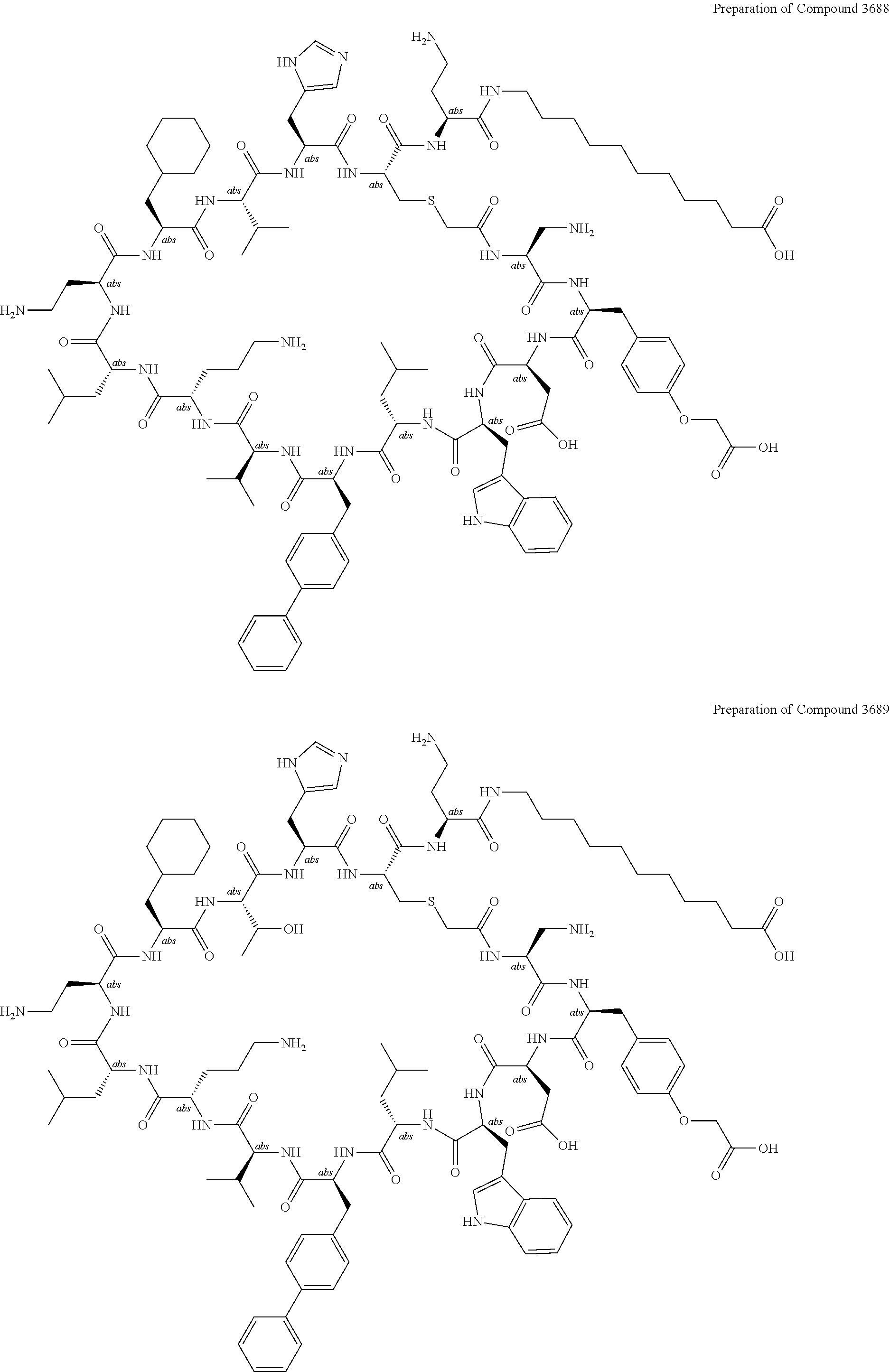

Compound 3688 was prepared on a 50 μmol scale. The yield of the product was 35.1 mg, and its estimated purity by LCMS analysis was 97%. Analysis condition A: Retention time=1.68 min; ESI-MS(+) m/z [M+2H]²⁺: 736.

Compound 3689 was prepared on a 50 μmol scale. The yield of the product was 54.8 mg, and its estimated purity by LCMS analysis was 95.8%. Analysis condition A: Retention time=1.71 min; ESI-MS(+) m/z [M+3H]³⁺: 737.1.

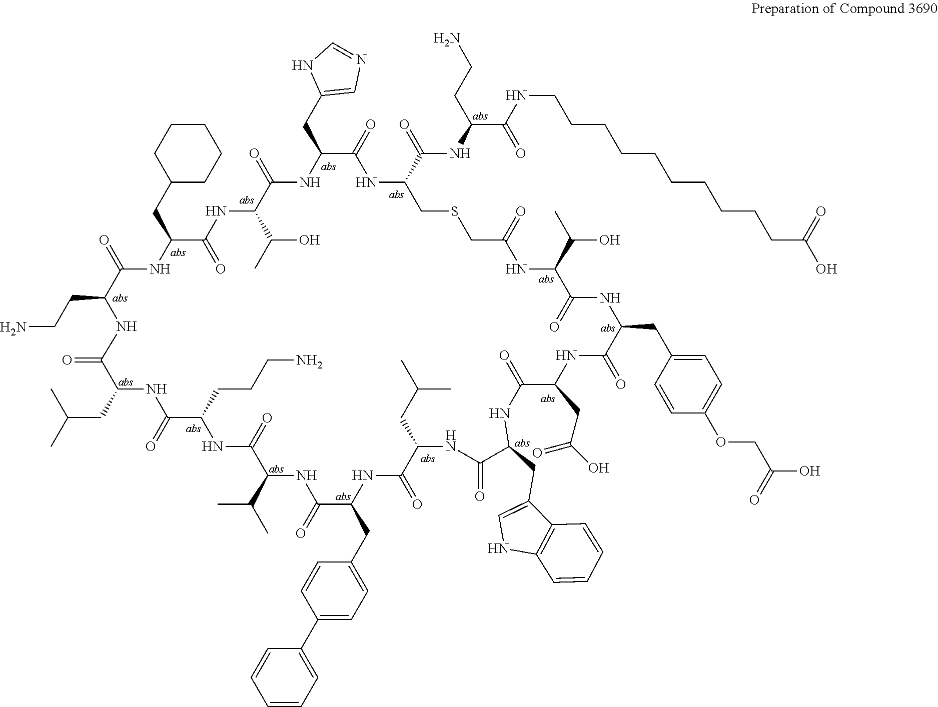 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](Cc2cnc[nH]2)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3690 was prepared on a 50 μmol scale. The yield of the product was 31.1 mg, and its estimated purity by LCMS analysis was 97%. Analysis condition A: Retention time=1.75 min; ESI-MS(+) m/z [M+2H]²⁺: 1112.1.

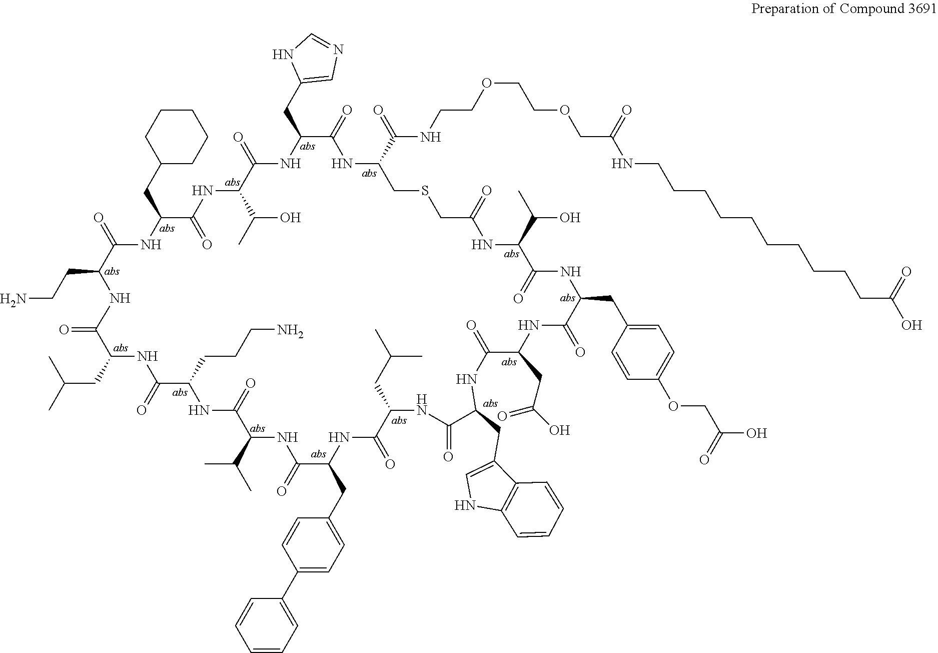 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC(=O)[C@H](Cc2cnc[nH]2)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3691 was prepared on a 50 μmol scale. The yield of the product was 5.2 mg, and its estimated purity by LCMS analysis was 97.3%. Analysis condition B: Retention time=1.63 min; ESI-MS(+) m/z [M+3H]³⁺: 756.3.

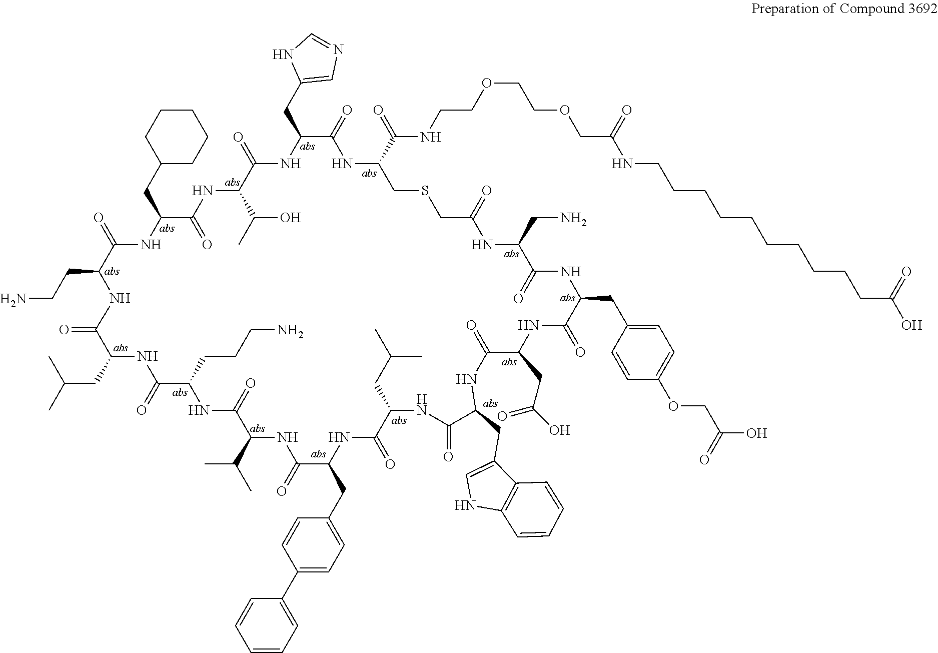 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCCOCCOCC(=O)NCCCCCCCCCCC(=O)O)NC(=O)[C@H](Cc2cnc[nH]2)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3692 was prepared on a 50 μmol scale. The yield of the product was 7.7 mg, and its estimated purity by LCMS analysis was 95.6%. Analysis condition B: Retention time=1.96 min; ESI-MS(+) m/z [M+3H]³⁺: 751.7.

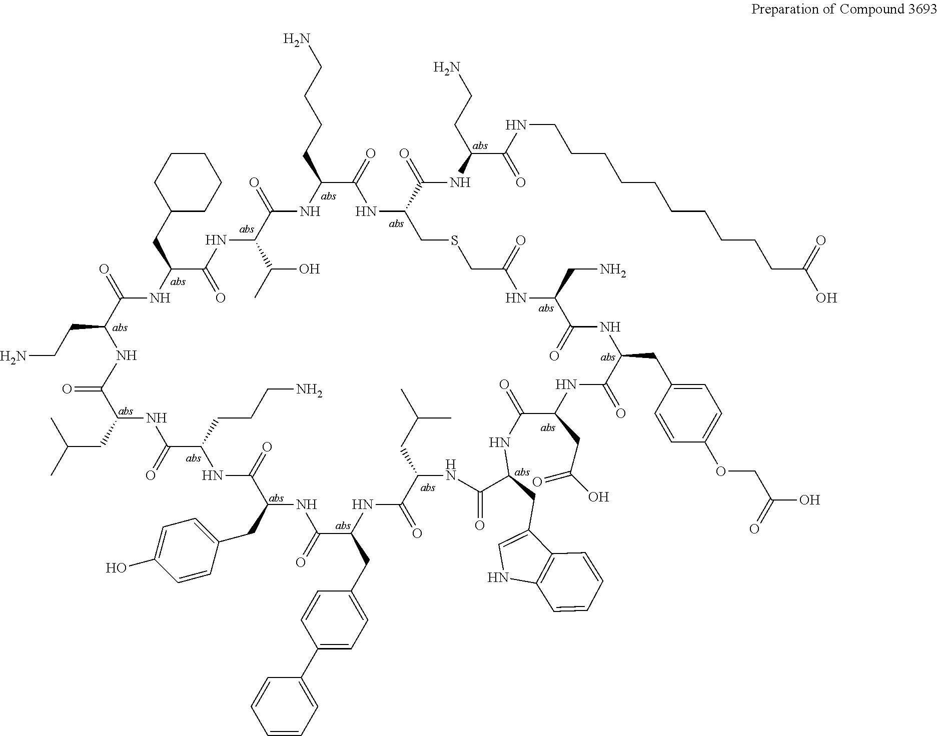 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCCCCCCCCCC(=O)O)NC(=O)[C@H](CCCCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 3693 was prepared on a 25 μmol scale. The yield of the product was 23.8 mg, and its estimated purity by LCMS analysis was 92.3%. Analysis condition A: Retention time=1.62 min; ESI-MS(+) m/z [M+3H]³⁺: 755.2.

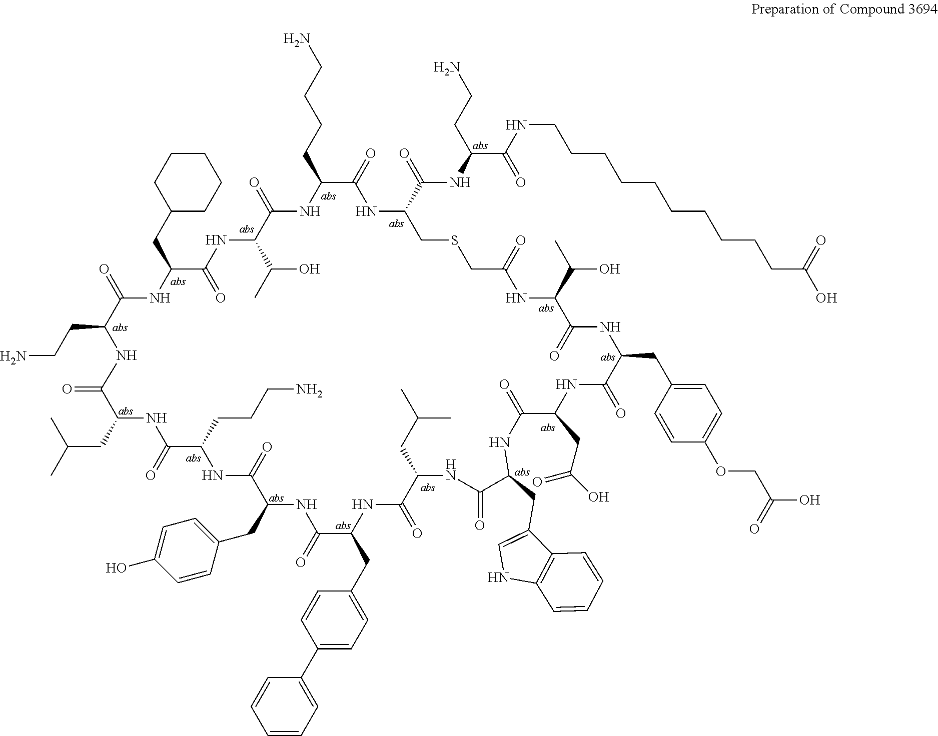 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CCCCN)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(O)cc2)NC(=O)[C@H](Cc2ccc(-c3ccccc3)cc2)NC1=O]

Compound 3694 was prepared on a 25 μmol scale. The yield of the product was 15.6 mg, and its estimated purity by LCMS analysis was 96.2%. Analysis condition A: Retention time=1.66 min; ESI-MS(+) m/z [M+3H]³⁺: 760.2.

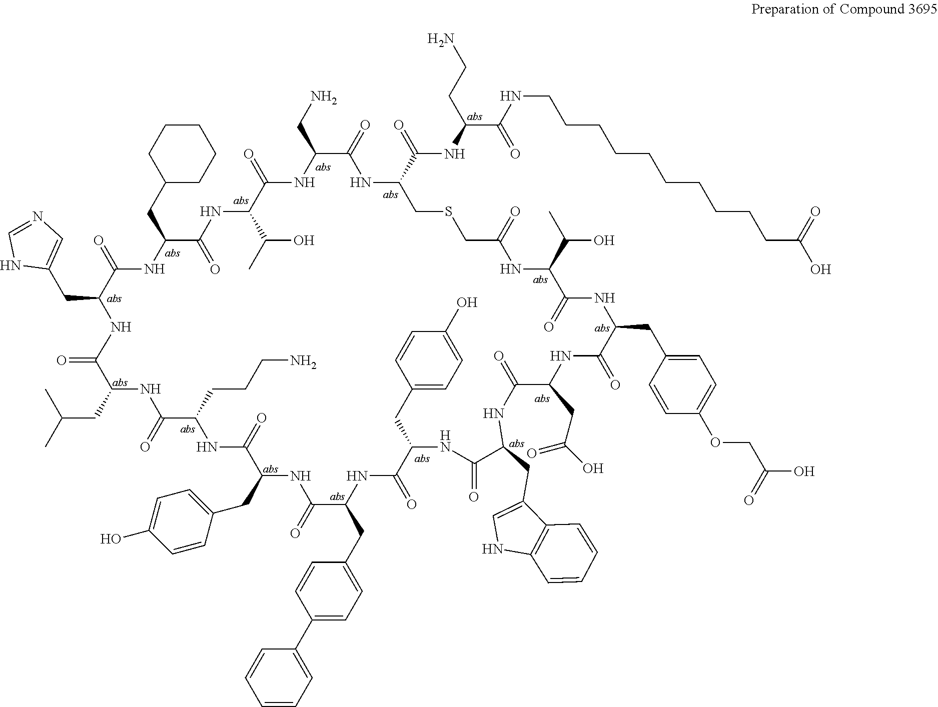 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](Cc2ccc(O)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CN)NC(=O)[C@H](C(C)O)NC(=O)[C@H](CC2CCCCC2)NC(=O)[C@H](Cc2cnc[nH]2)NC1=O]

Compound 3695 was prepared on a 25 μmol scale. The yield of the product was 16.5 mg, and its estimated purity by LCMS analysis was 96.9%. Analysis condition B: Retention time=1.58 min; ESI-MS(+) m/z [M+2H]²⁺: 1162.2.

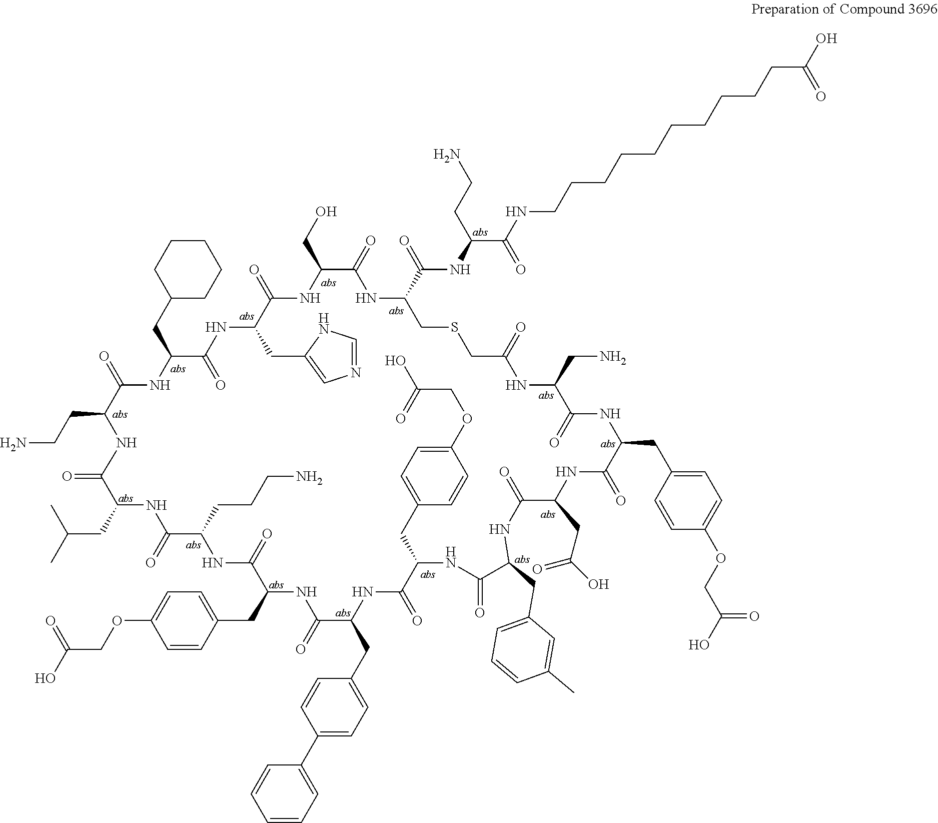 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3696 was prepared on a 50 μmol scale. The yield of the product was 1.6 mg, and its estimated purity by LCMS analysis was 95.6%. Analysis condition A: Retention time=1.56, 1.6 min; ESI-MS(+) m/z [M+2H]²⁺: 1200.1, 1200.1.

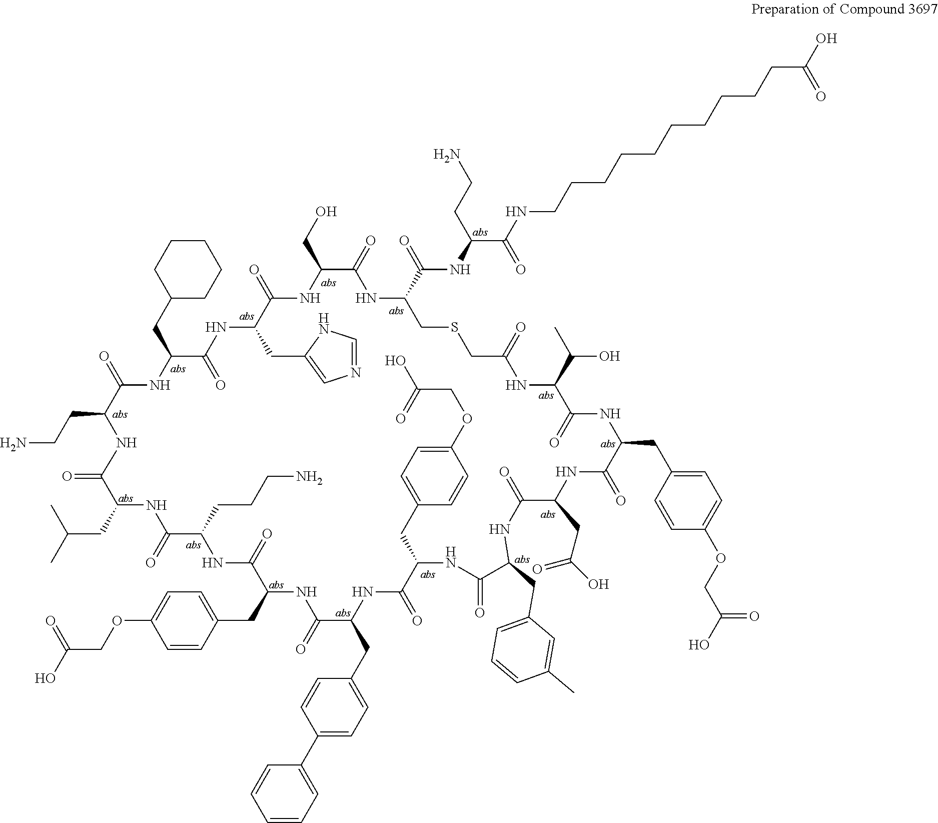 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3697 was prepared on a 50 μmol scale. The yield of the product was 3.7 mg, and its estimated purity by LCMS analysis was 96.5%. Analysis condition A: Retention time=1.6 min; ESI-MS(+) m/z [M+3H]³⁺: 805.2.

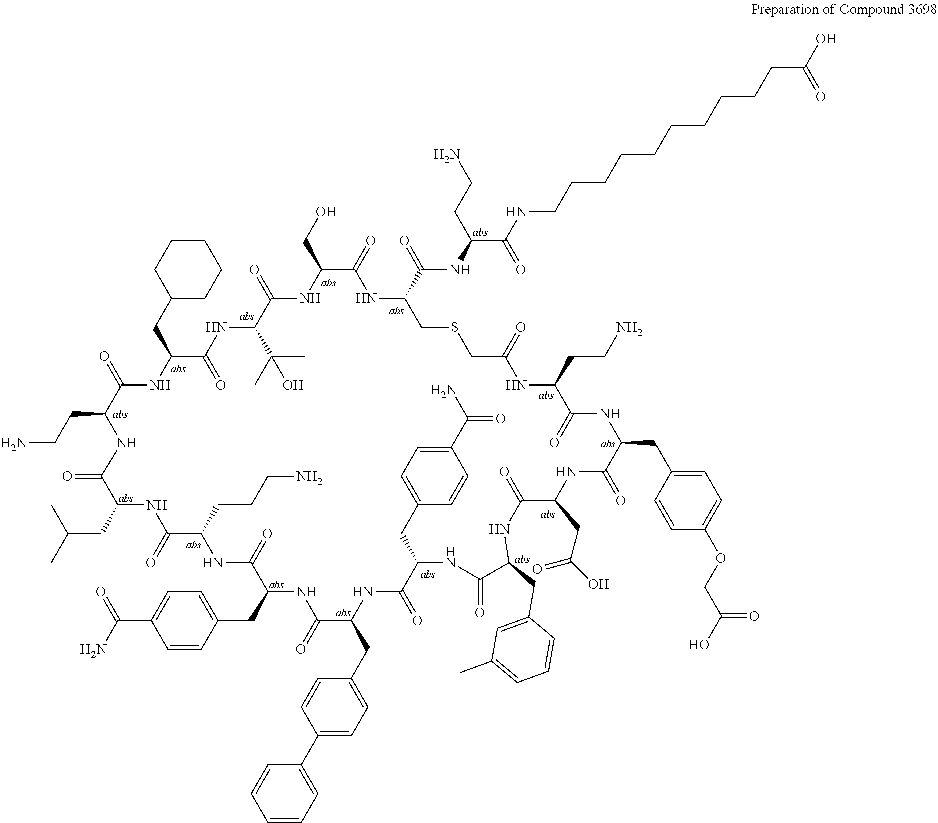 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(N)=O)cc3)NC(=O)[C@H](Cc3ccc(-c4ccccc4)cc3)NC(=O)[C@H](Cc3ccc(C(N)=O)cc3)NC2=O)c1]

Compound 3698 was prepared on a 50 μmol scale. The yield of the product was 22.9 mg, and its estimated purity by LCMS analysis was 97.7%. Analysis condition B: Retention time=1.52 min; ESI-MS(+) m/z [M+3H]³⁺: 777.1.

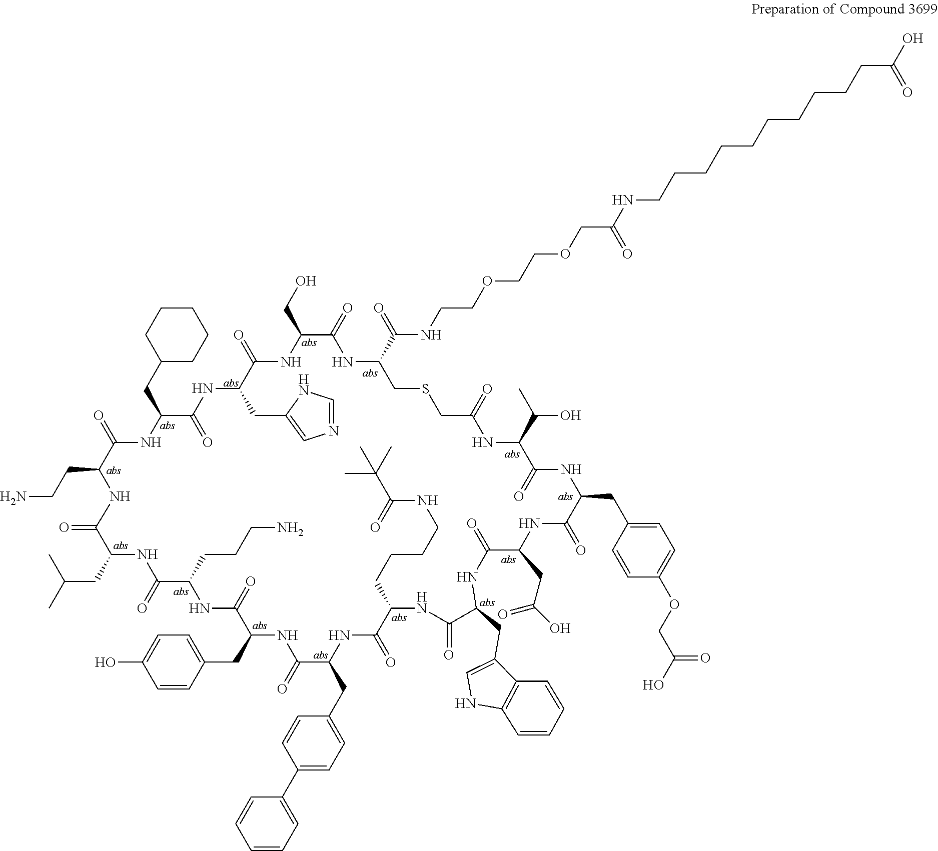 [SMILES: CC(C)C[C@H]1NC(=O)[C@H](CCCN)NC(=O)[C@H](Cc2ccc(O)cc2)NC(=O)[C@H](Cc2ccc(-c3ccccc3)cc2)NC(=O)[C@H](CCCCNC(=O)C(C)(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC(=O)[C@H](CO)NC(=O)[C@H](Cc2cnc[nH]2)NC(=O)[C@H](CC2CCCCC2)NC(=O)[C@H](CCN)NC1=O]

Compound 3699 was prepared on a 50 μmol scale. The yield of the product was 1.1 mg, and its estimated purity by LCMS analysis was 100%. Analysis condition A: Retention time=1.66 min; ESI-MS(+) m/z [M+3H]³⁺: 806.2.

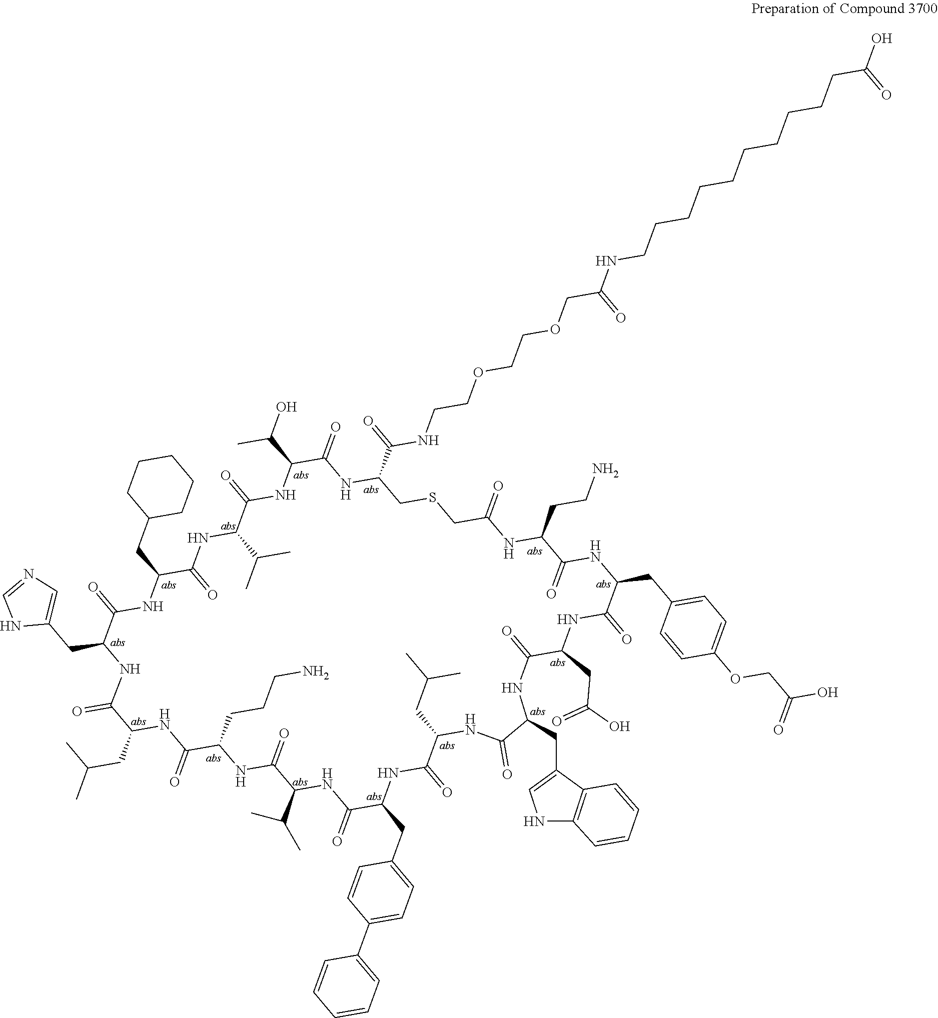 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)NC(=O)CSC[C@@H](C(=O)NCCOCCOCC(=O)NCCCCCCCCCCC(=O)O)NC(=O)[C@H](C(C)O)NC(=O)[C@H](C(C)C)NC(=O)[C@H](CC2CCCCC2)NC(=O)[C@H](Cc2cnc[nH]2)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3700 was prepared on a 50 μmol scale. The yield of the product was 44.7 mg, and its estimated purity by LCMS analysis was 95.3%. Analysis condition B: Retention time=1.71 min; ESI-MS(+) m/z [M+2H]²⁺: 1133.3.

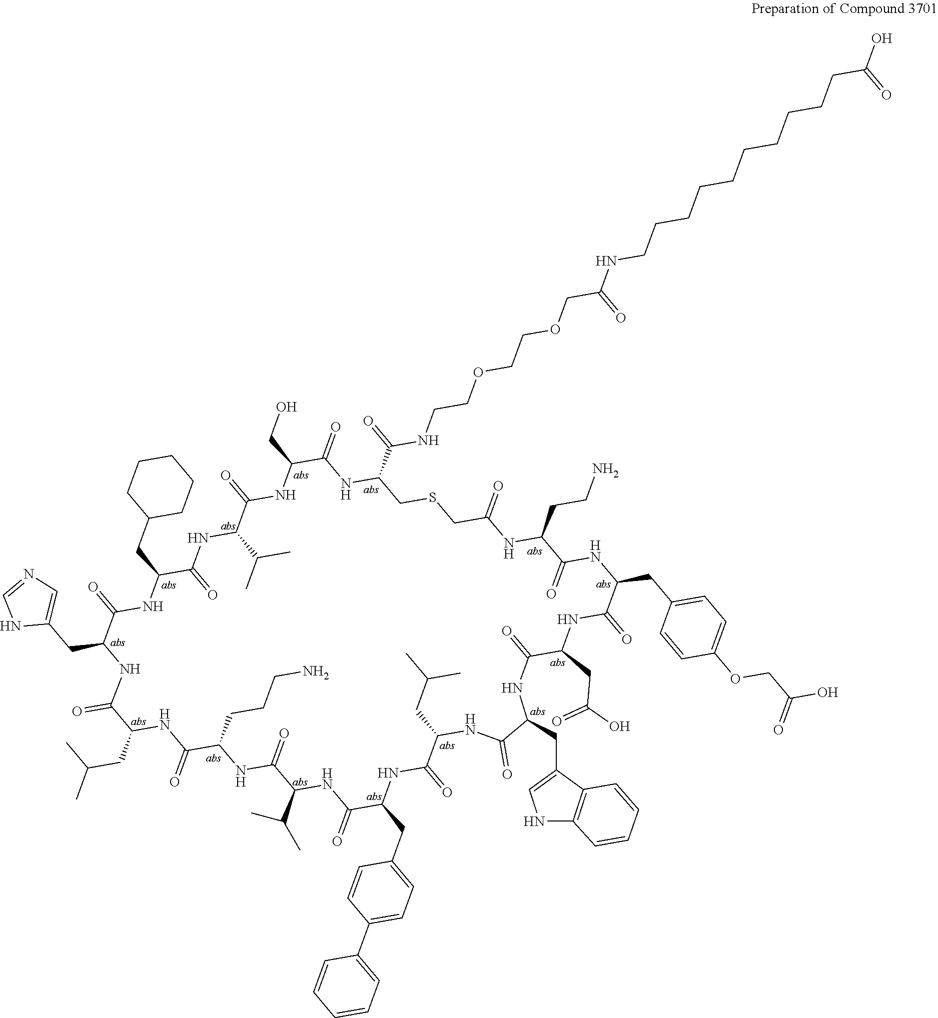 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)NC(=O)CSC[C@@H](C(=O)NCCOCCOCC(=O)NCCCCCCCCCCC(=O)O)NC(=O)[C@H](CO)NC(=O)[C@H](C(C)C)NC(=O)[C@H](CC2CCCCC2)NC(=O)[C@H](Cc2cnc[nH]2)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 3701 was prepared on a 50 μmol scale. The yield of the product was 28.7 mg, and its estimated purity by LCMS analysis was 97.3%. Analysis condition A: Retention time=1.77 min; ESI-MS(+) m/z [M+3H]³⁺: 751.8.

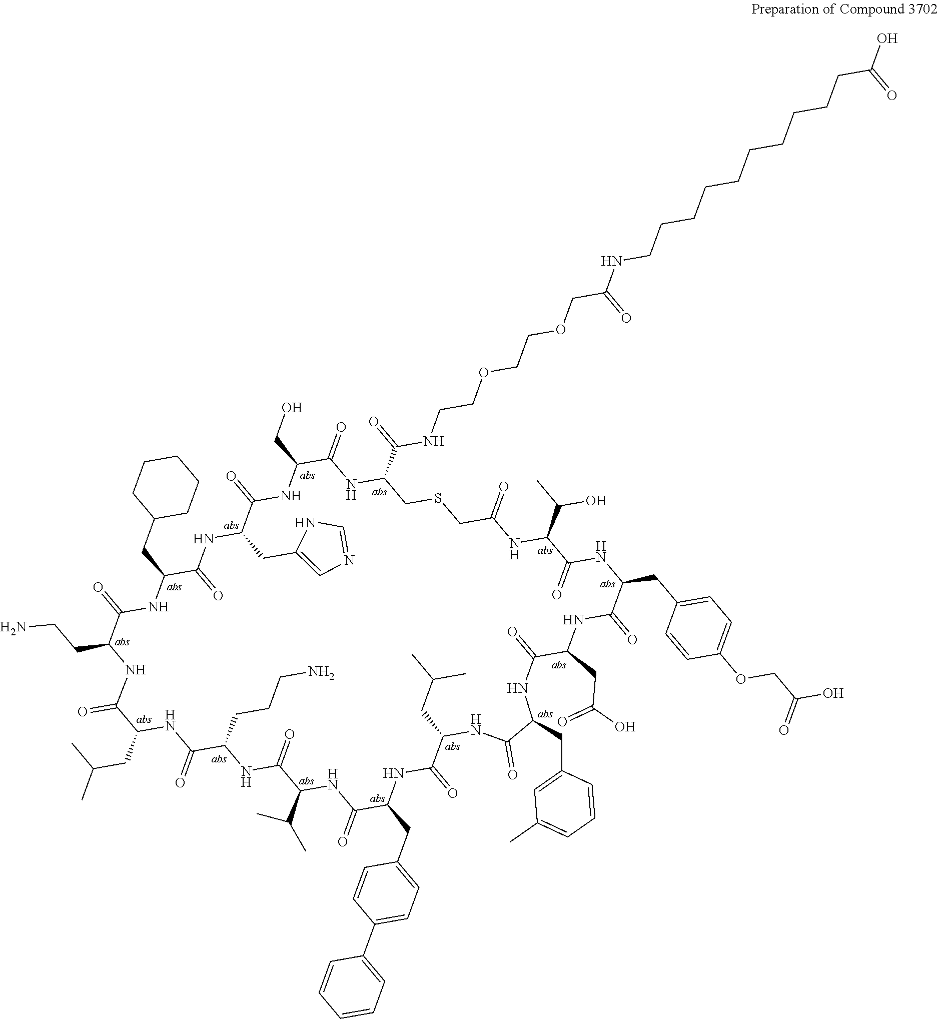 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 3702 was prepared on a 50 μmol scale. The yield of the product was 0.9 mg, and its estimated purity by LCMS analysis was 100%. Analysis condition A: Retention time=1.76 min; ESI-MS(+) m/z [M+2H]²⁺: 1108.2.

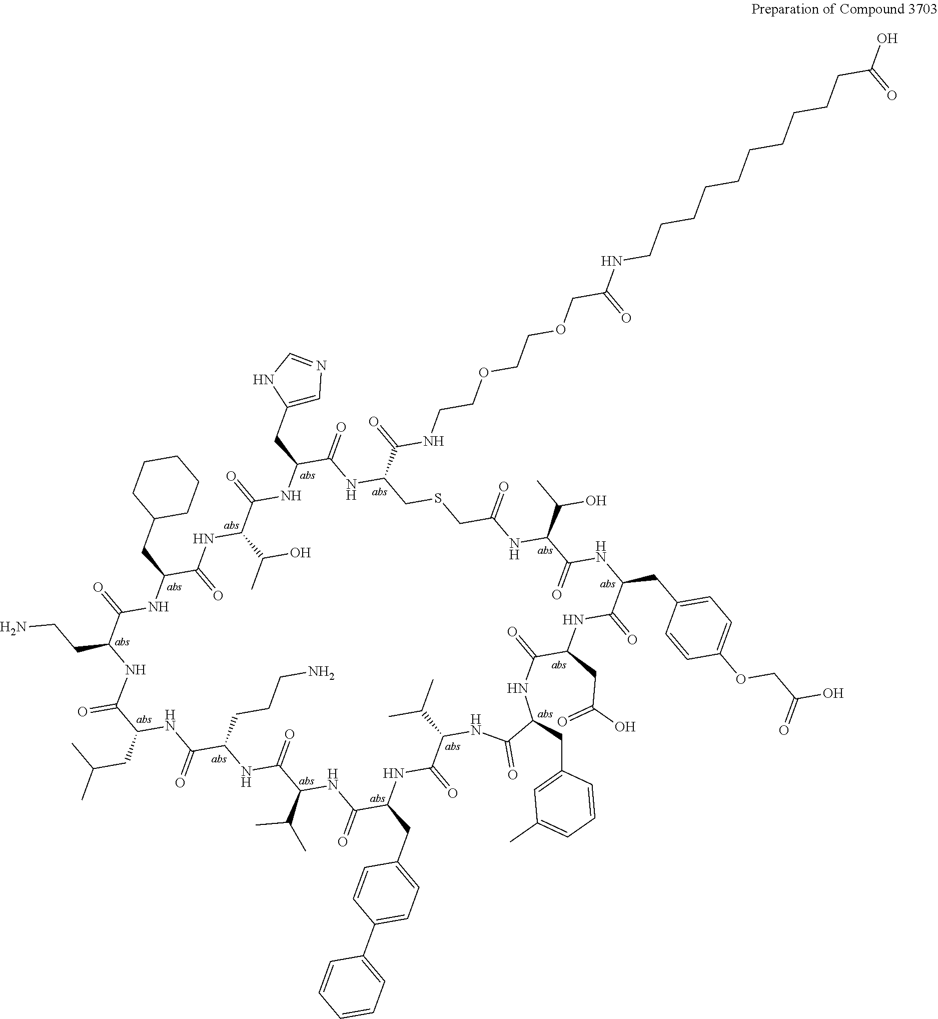 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)NCCOCCOCC(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 3703 was prepared on a 50 μmol scale. The yield of the product was 7.3 mg, and its estimated purity by LCMS analysis was 95.7%. Analysis condition A: Retention time=1.86 min; ESI-MS(+) m/z [M+2H]²⁺: 1115.1.

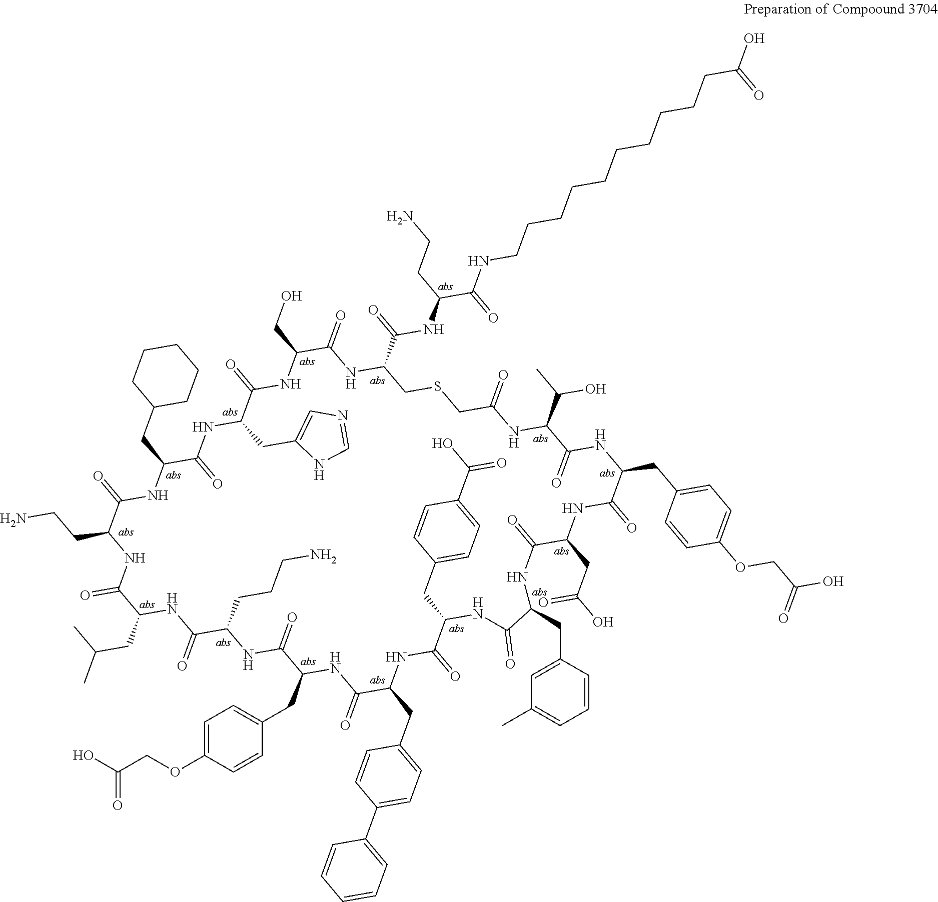 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3704 was prepared on a 50 μmol scale. The yield of the product was 14.1 mg, and its estimated purity by LCMS analysis was 97.3%. Analysis condition A: Retention time=1.41 min; ESI-MS(+) m/z [M+2H]²⁺: 1192.9.

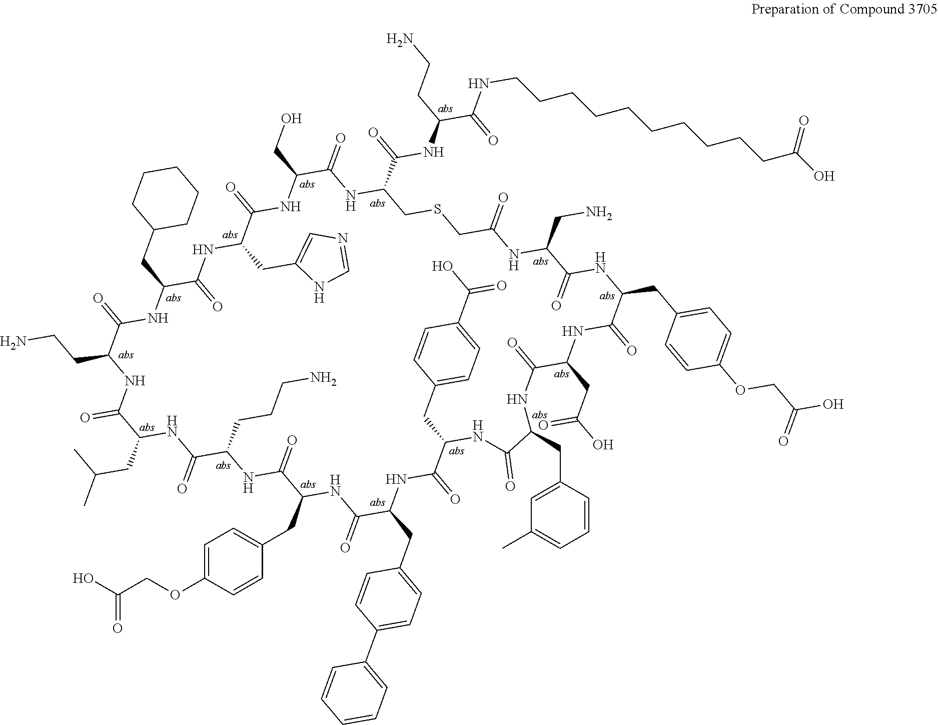 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3705 was prepared on a 50 μmol scale. The yield of the product was 7.9 mg, and its estimated purity by LCMS analysis was 91.1%. Analysis condition A: Retention time=1.47 min; ESI-MS(+) m/z [M+2H]²⁺: 1184.6.

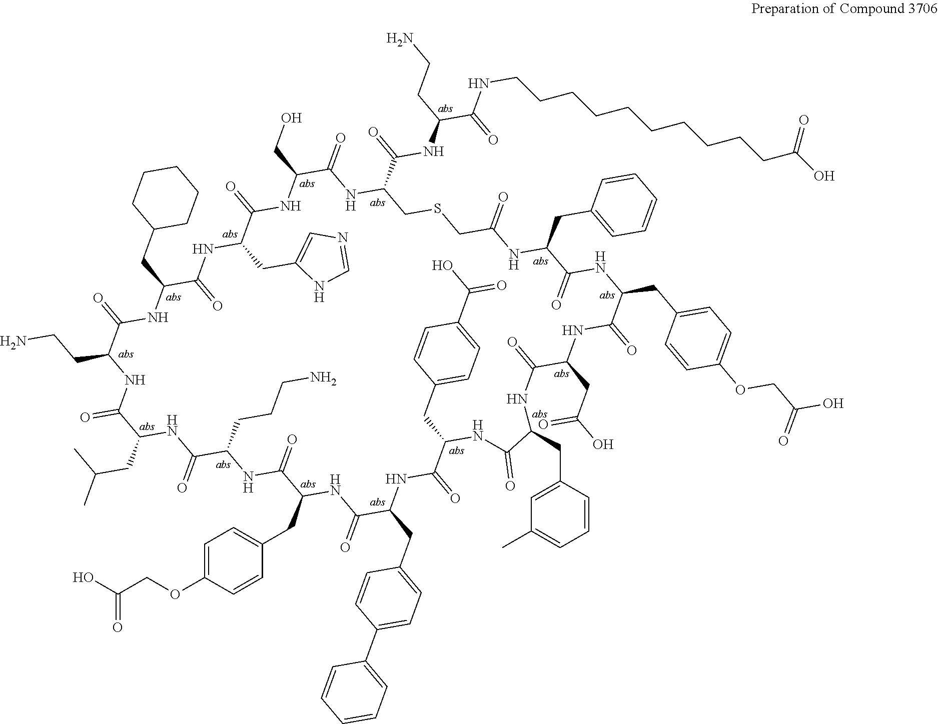 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccccc3)NC(=O)CSC[C@@H](C(=O)N[C@@H](CCN)C(=O)NCCCCCCCCCCC(=O)O)NC(=O)[C@H](CO)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3706 was prepared on a 50 μmol scale. The yield of the product was 16.9 mg, and its estimated purity by LCMS analysis was 95.1%. Analysis condition A: Retention time=1.62 min; ESI-MS(+) m/z [M+2H]²⁺: 1215.2.

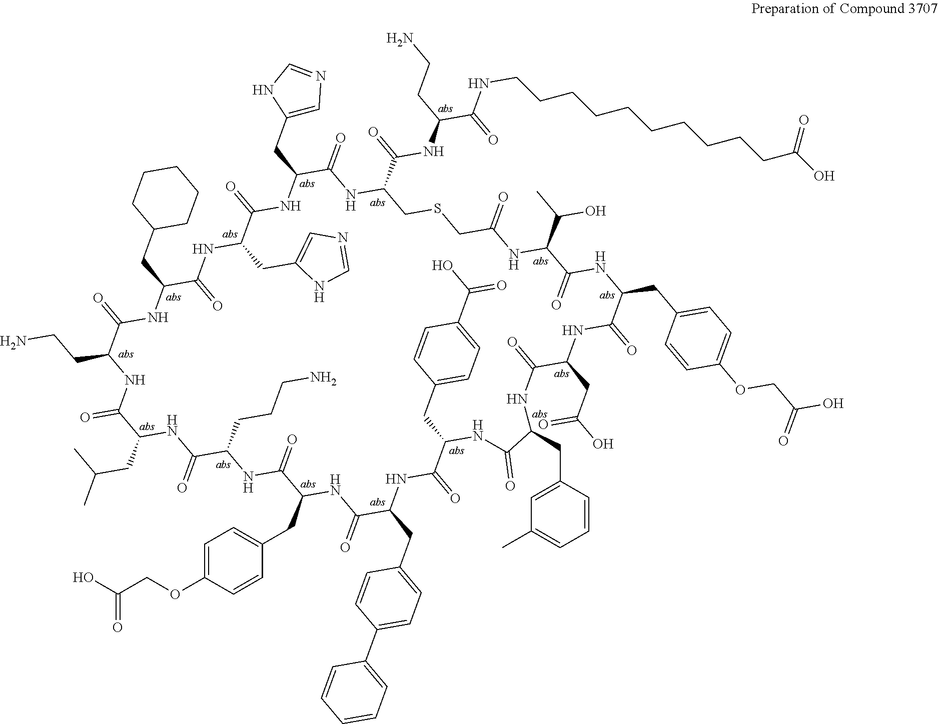 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](Cc3cnc[nH]3)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3707 was prepared on a 50 μmol scale. The yield of the product was 23.9 mg, and its estimated purity by LCMS analysis was 97.3%. Analysis condition B: Retention time=1.66 min; ESI-MS(+) m/z [M+3H]³⁺: 800.2.

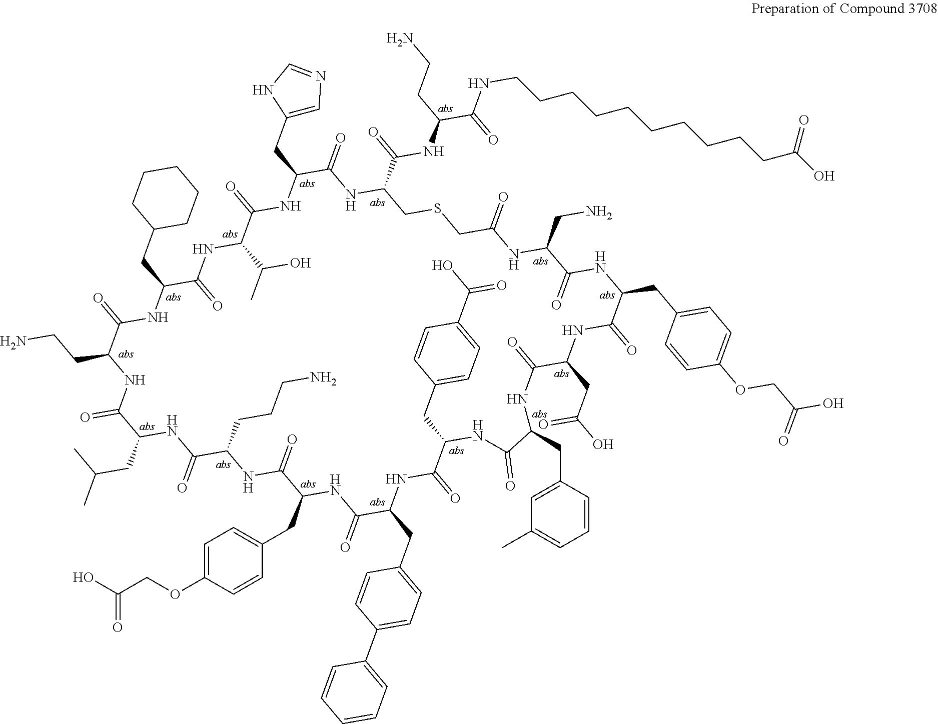 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3708 was prepared on a 50 μmol scale. The yield of the product was 21.8 mg, and its estimated purity by LCMS analysis was 100%. Analysis condition A: Retention time=1.42 min; ESI-MS(+) m/z [M+3H]³⁺: 795.

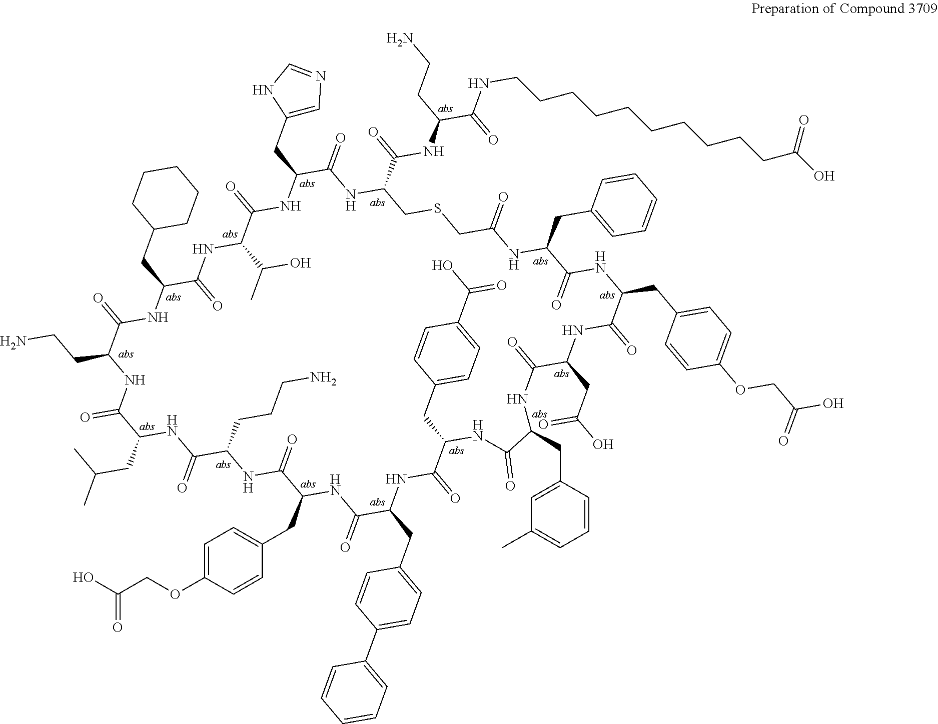 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccccc3)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3709 was prepared on a 50 μmol scale. The yield of the product was 71.3 mg, and its estimated purity by LCMS analysis was 91.7%. Analysis condition A: Retention time-1.6 min; ESI-MS(+) m/z [M+3H]³⁺: 815.1.

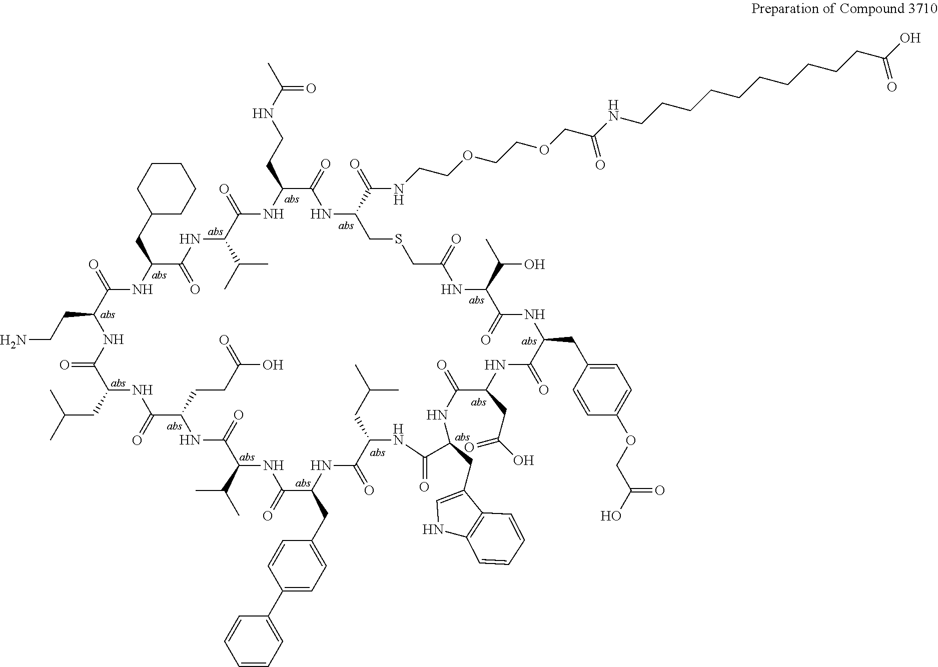 [SMILES: CC(=O)NCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCC(=O)O)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)NCCOCCOCC(=O)NCCCCCCCCCCC(=O)O)NC1=O]

Compound 3710 was prepared on a 50 μmol scale. The yield of the product was 4.4 mg, and its estimated purity by LCMS analysis was 95.8%. Analysis condition B: Retention time=2.02 min; ESI-MS(+) m/z [M+2H]²⁺: 1144.

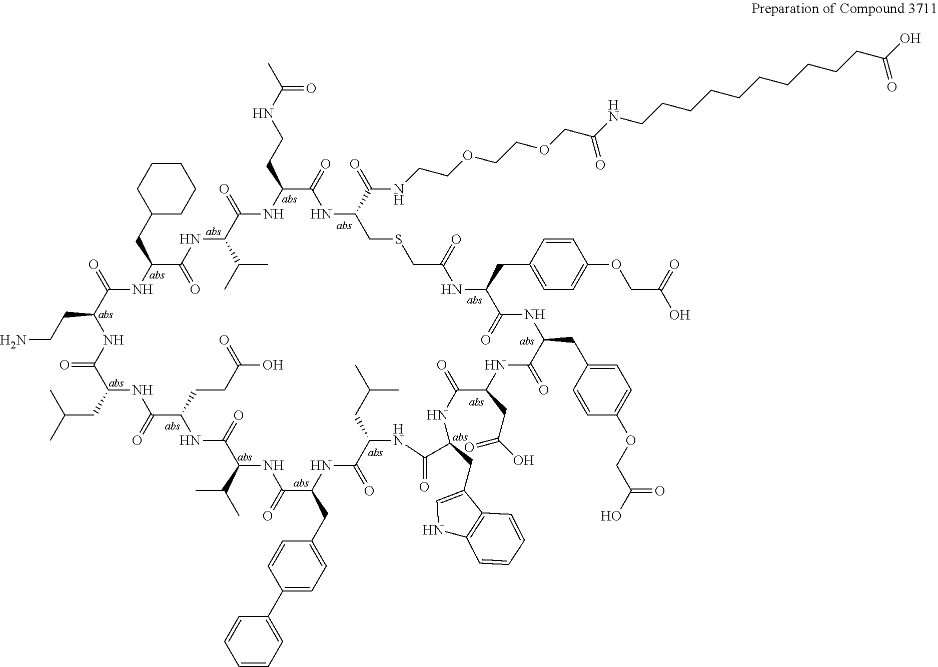 [SMILES: CC(=O)NCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCC(=O)O)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCCOCCOCC(=O)NCCCCCCCCCCC(=O)O)NC1=O]

Compound 3711 was prepared on a 50 μmol scale. The yield of the product was 12.1 mg, and its estimated purity by LCMS analysis was 95.3%. Analysis condition B: Retention time=2.05, 2.09 min; ESI-MS(+) m/z [M+2H]²⁺: 1204, 802.94.

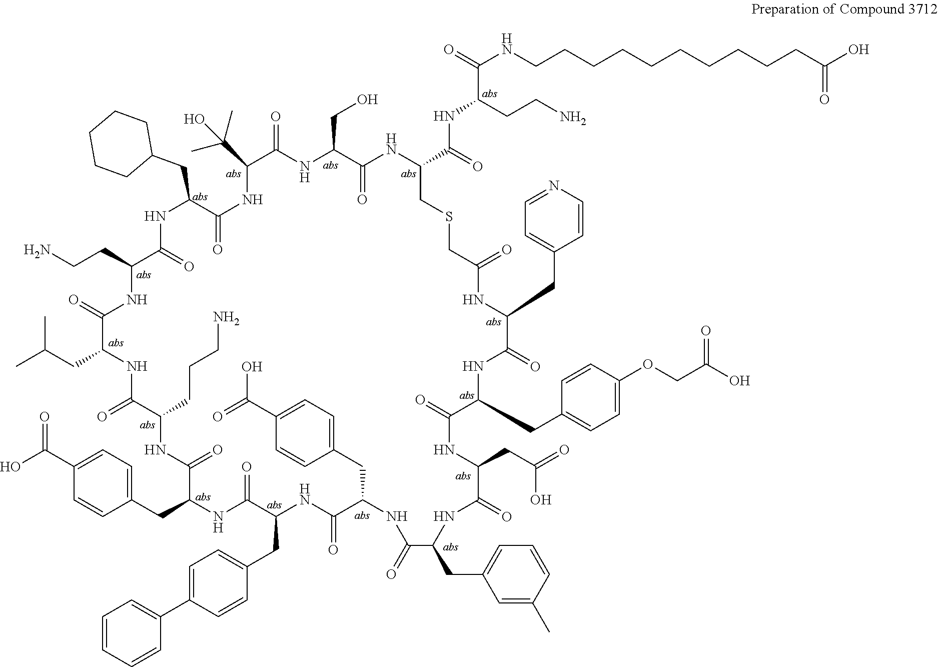 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3712 was prepared on a 50 μmol scale. The yield of the product was 0.8 mg, and its estimated purity by LCMS analysis was 98.1%. Analysis condition B: Retention time=1.58 min; ESI-MS(+) m/z [M+2H]²⁺: 1190.1.

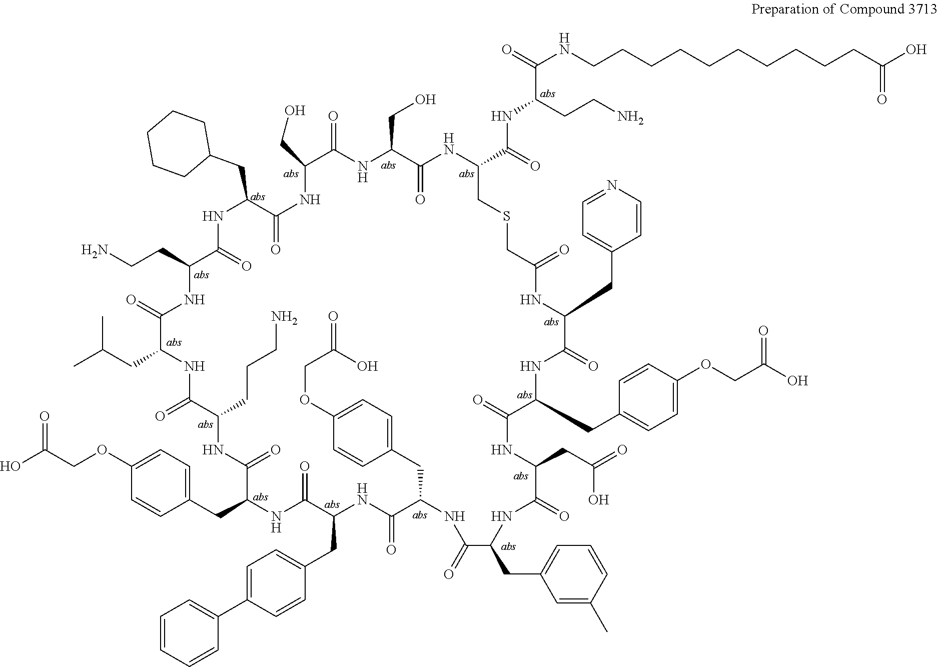 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3713 was prepared on a 50 μmol scale. The yield of the product was 3.2 mg, and its estimated purity by LCMS analysis was 95.4%. Analysis condition B: Retention time=1.52 min; ESI-MS(+) m/z [M+2H]²⁺: 1206.7.

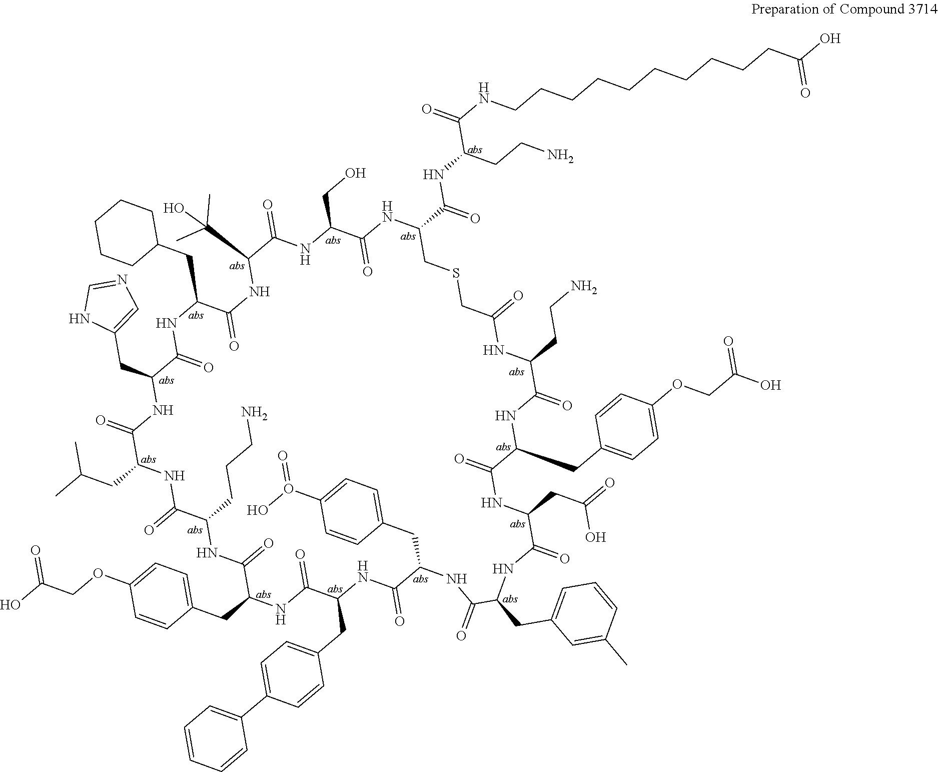

Compound 3714 was prepared on a 50 μmol scale. The yield of the product was 9.4 mg, and its estimated purity by LCMS analysis was 96.3%. Analysis condition A: Retention time=1.56 min; ESI-MS(+) m/z [M+3H]³⁺: 800.1.

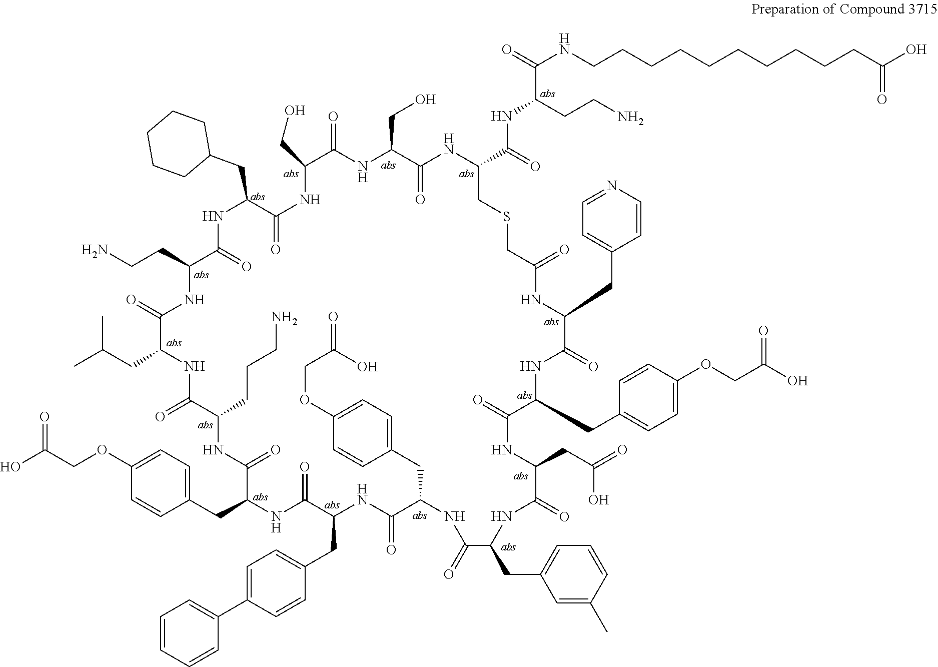 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](CO)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3715 was prepared on a 50 μmol scale. The yield of the product was 1.7 mg, and its estimated purity by LCMS analysis was 100%. Analysis condition A: Retention time=1.47 min; ESI-MS(+) m/z [M+2H]²⁺: 1206.2.

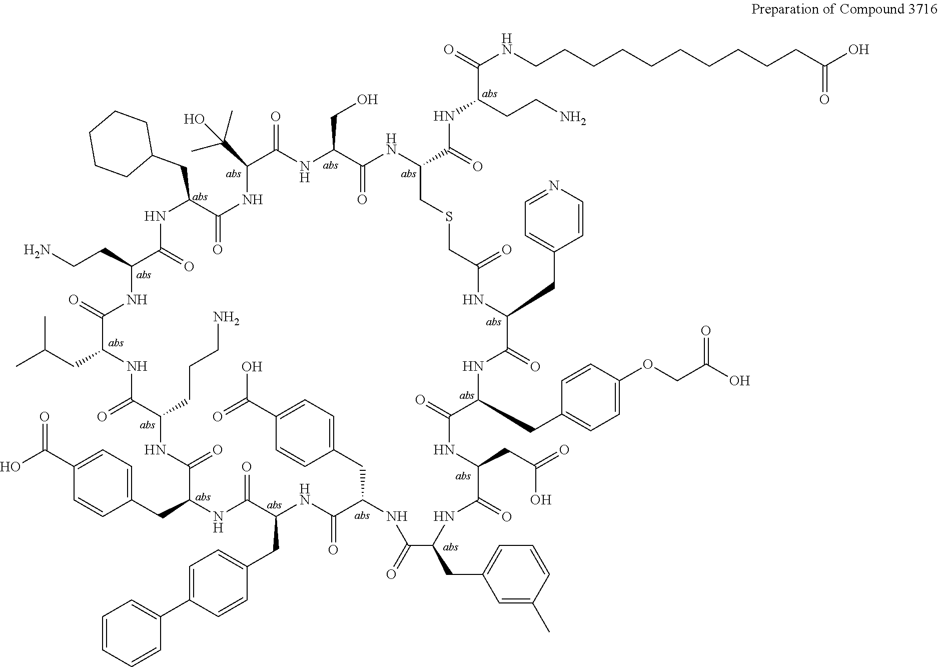 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3716 was prepared on a 50 μmol scale. The yield of the product was 2.4 mg, and its estimated purity by LCMS analysis was 96.8%. Analysis condition B: Retention time=1.59 min; ESI-MS(+) m/z [M+2H]²⁺: 1189.8.

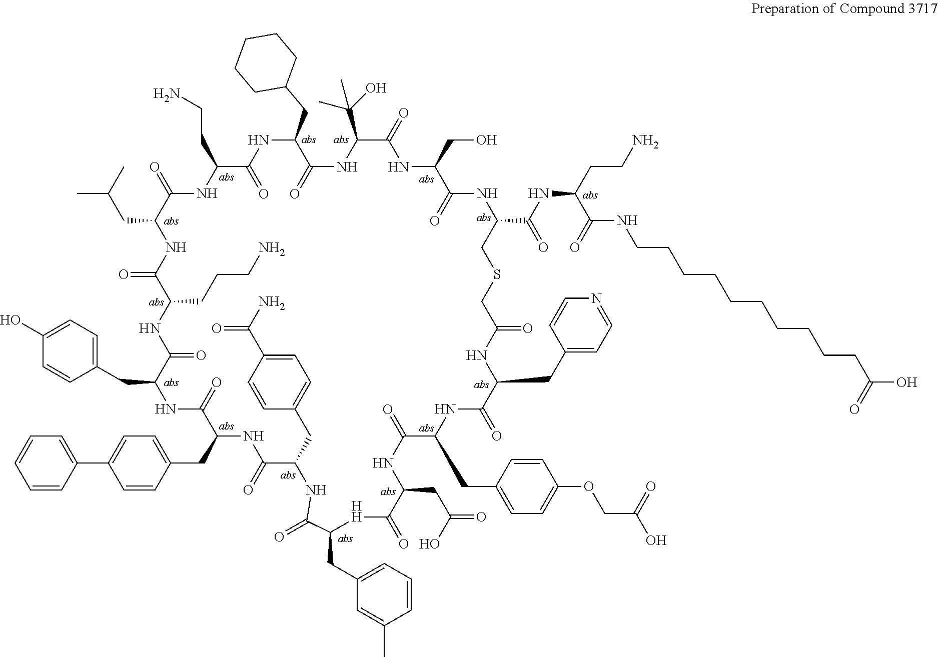

Compound 3717 was prepared on a 30 μmol scale. The yield of the product was 0.9 mg, and its estimated purity by LCMS analysis was 98.9%. Analysis condition A: Retention time=1.57 min; ESI-MS(+) m/z [M+2H]²⁺: 1175.4.

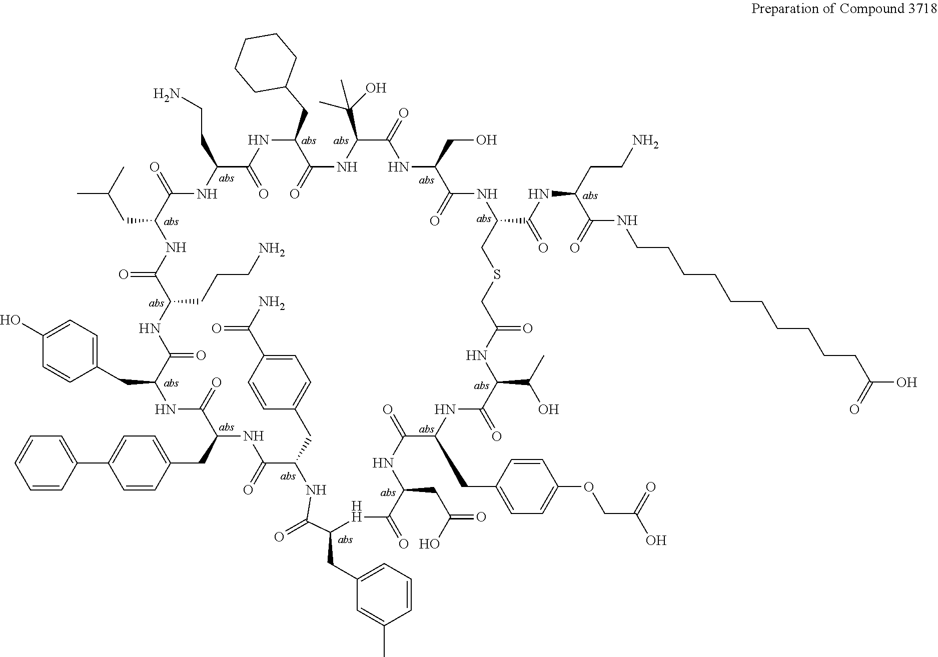

Compound 3718 was prepared on a 30 μmol scale. The yield of the product was 5.2 mg, and its estimated purity by LCMS analysis was 100%. Analysis condition A: Retention time=1.65 min; ESI-MS(+) m/z [M+2H]²⁺: 1152.1.

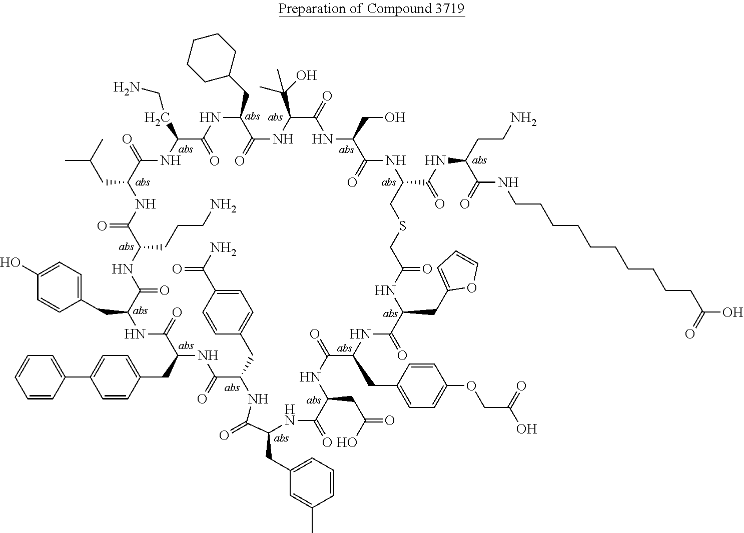 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccco3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 3719 was prepared on a 30 μmol scale. The yield of the product was 9 mg, and its estimated purity by LCMS analysis was 97.6%. Analysis condition A: Retention time=1.71 min; ESI-MS(+) m/z [M+2H]²⁺: 1170.

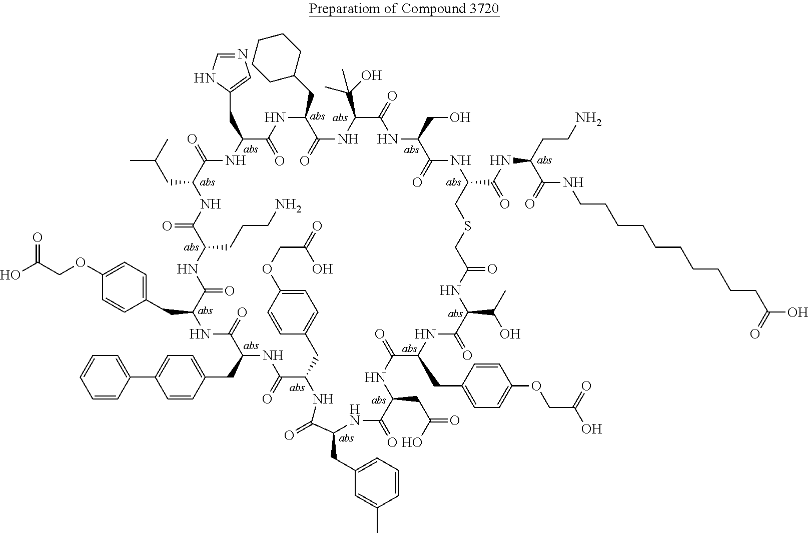 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3720 was prepared on a 30 μmol scale. The yield of the product was 0.7 mg, and its estimated purity by LCMS analysis was 95.1%. Analysis condition A: Retention time=1.46 min; ESI-MS(+) m/z [M+2H]²⁺: 1215.3.

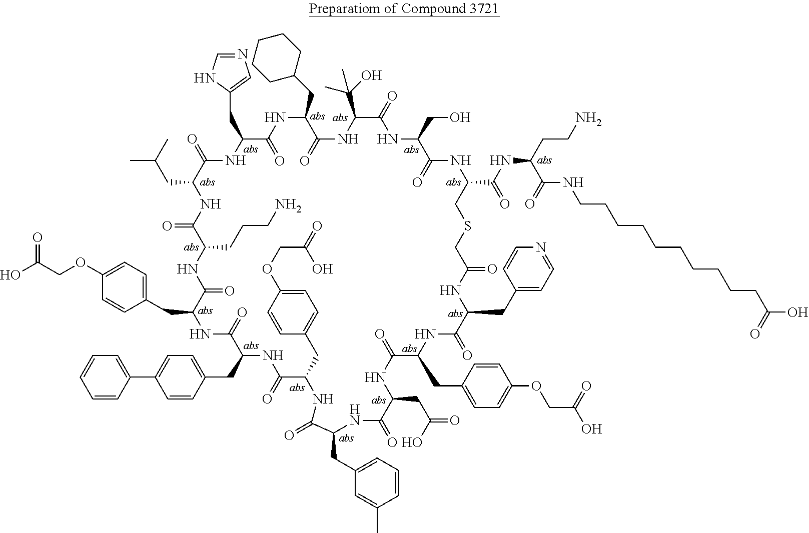 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3721 was prepared on a 30 μmol scale. The yield of the product was 1.5 mg, and its estimated purity by LCMS analysis was 96.3%. Analysis condition A: Retention time=1.5, 1.54 min; ESI-MS(+) m/z [M+3H]³⁺: 826.

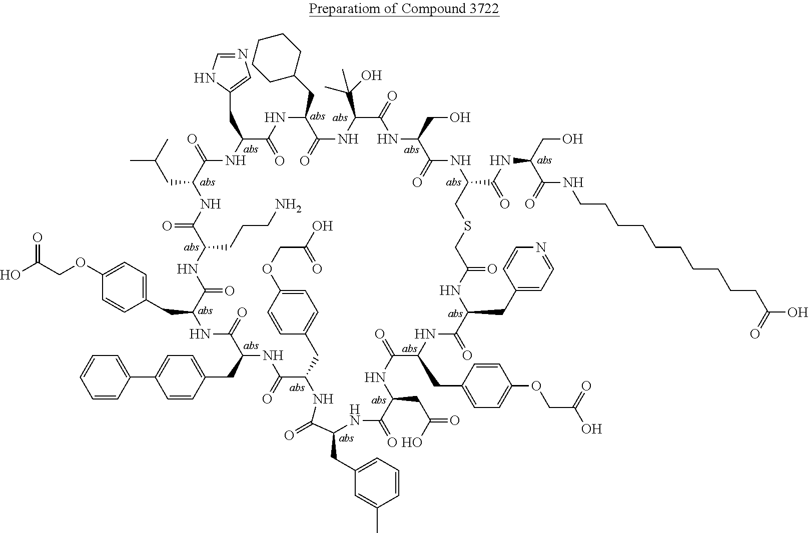 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CO)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3722 was prepared on a 30 μmol scale. The yield of the product was 4.8 mg, and its estimated purity by LCMS analysis was 100%. Analysis condition A: Retention time-1.37 min; ESI-MS(+) m/z [M+2H]²⁺: 1231.9.

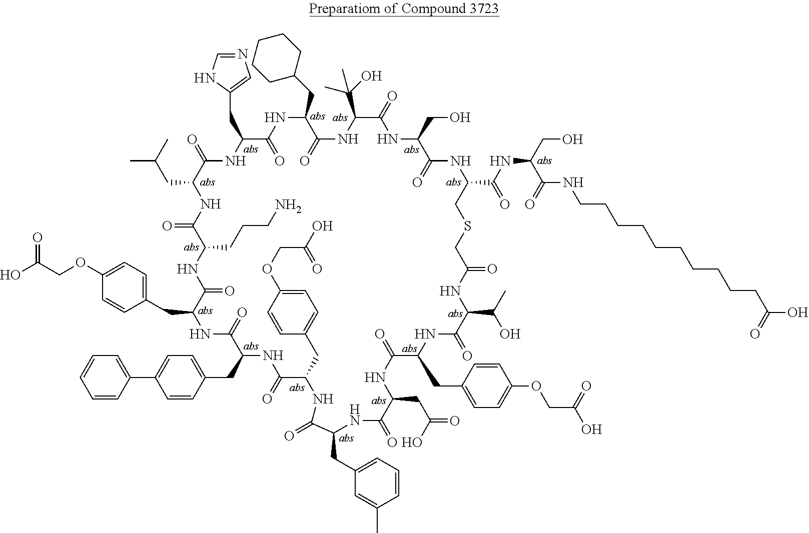 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CO)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3723 was prepared on a 30 μmol scale. The yield of the product was 2.6 mg, and its estimated purity by LCMS analysis was 95.3%. Analysis condition B: Retention time=1.71 min; ESI-MS(+) m/z [M+2H]²⁺: 1208.2.

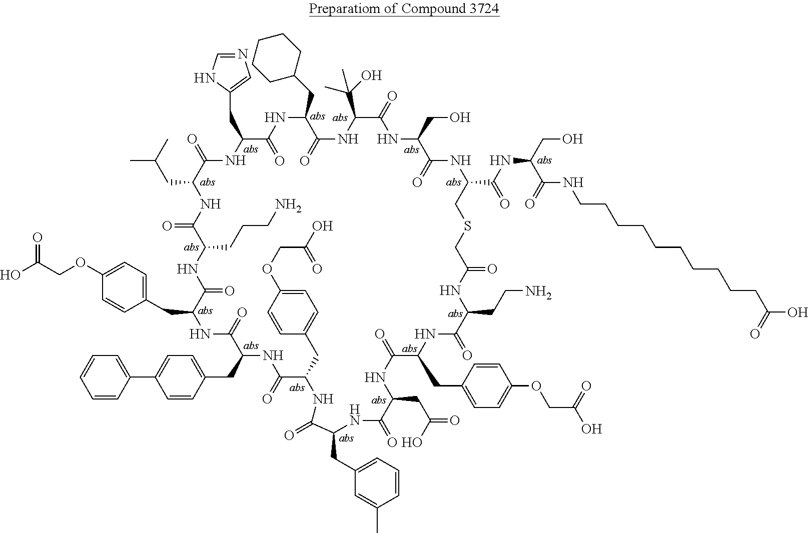 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CO)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3724 was prepared on a 30 μmol scale. The yield of the product was 14.2 mg, and its estimated purity by LCMS analysis was 91.5%. Analysis condition A: Retention time=1.41, 1.45 min; ESI-MS(+) m/z [M+2H]²⁺: 1208.

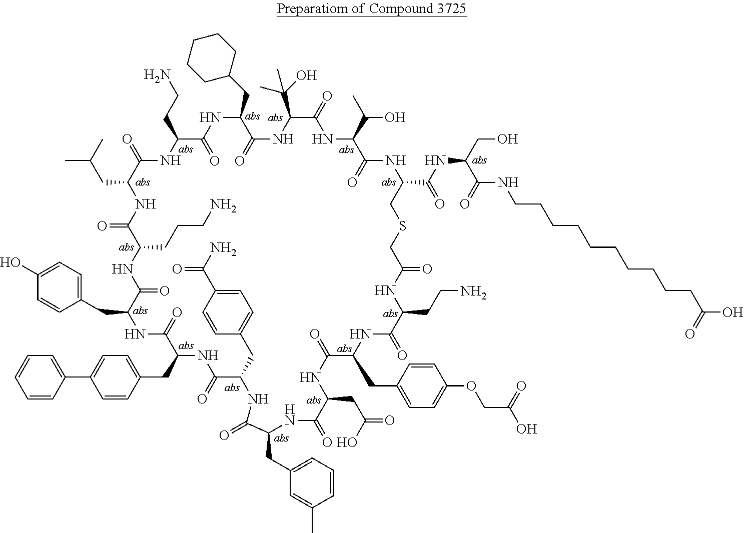 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CO)C(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 3725 was prepared on a 30 μmol scale. The yield of the product was 6.3 mg, and its estimated purity by LCMS analysis was 100%. Analysis condition A: Retention time=1.62 min; ESI-MS(+) m/z [M+2H]²⁺: 1152.3.

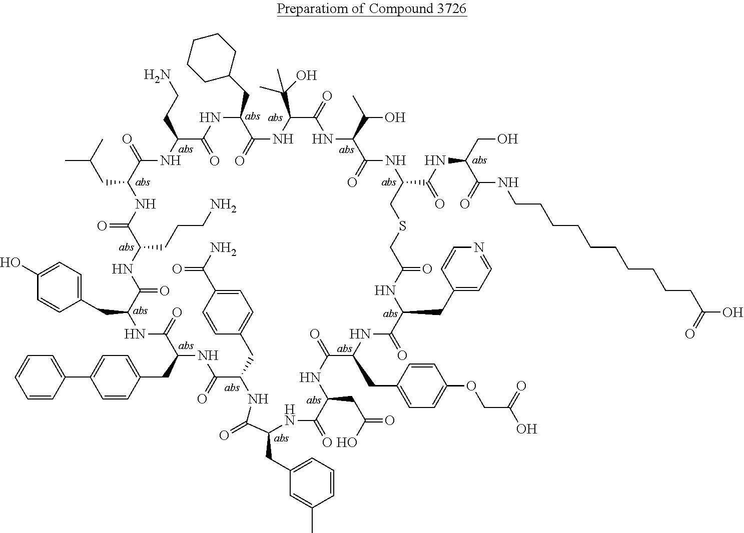 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CO)C(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 3726 was prepared on a 30 μmol scale. The yield of the product was 4.3 mg, and its estimated purity by LCMS analysis was 95.8%. Analysis condition A: Retention time=1.57 min; ESI-MS(+) m/z [M+2H]²⁺: 1176.1.

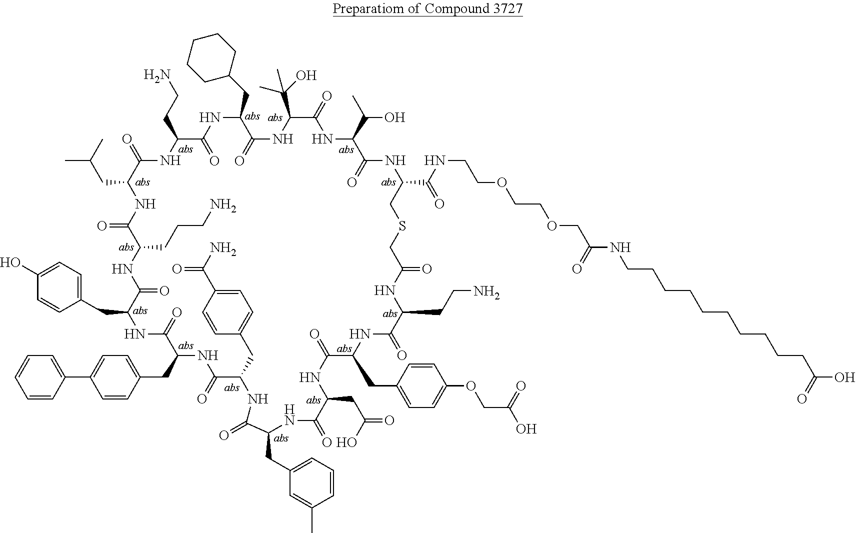 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)NCCOCCOCC(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 3727 was prepared on a 30 μmol scale. The yield of the product was 10.9 mg, and its estimated purity by LCMS analysis was 96.2%. Analysis condition A: Retention time=1.6 min; ESI-MS(+) m/z [M+2H]²⁺: 1181.2.

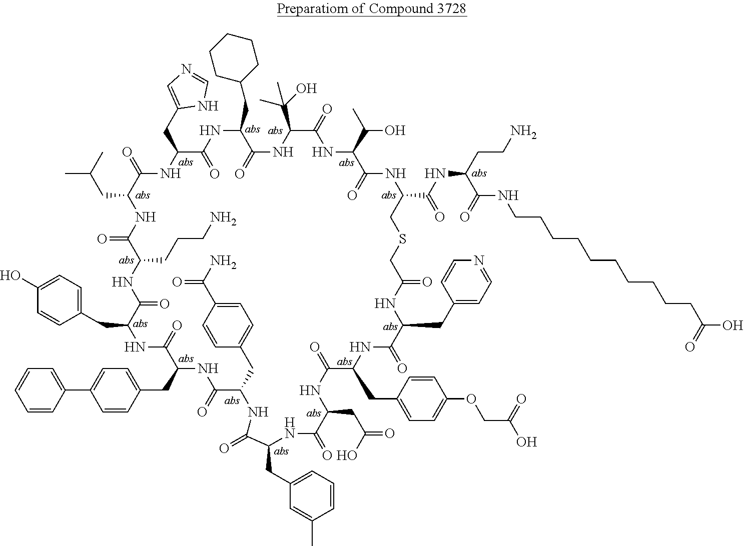 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 3728 was prepared on a 30 μmol scale. The yield of the product was 26.5 mg, and its estimated purity by LCMS analysis was 97%. Analysis condition A: Retention time=1.64 min; ESI-MS(+) m/z [M+2H]²⁺: 1201.1.

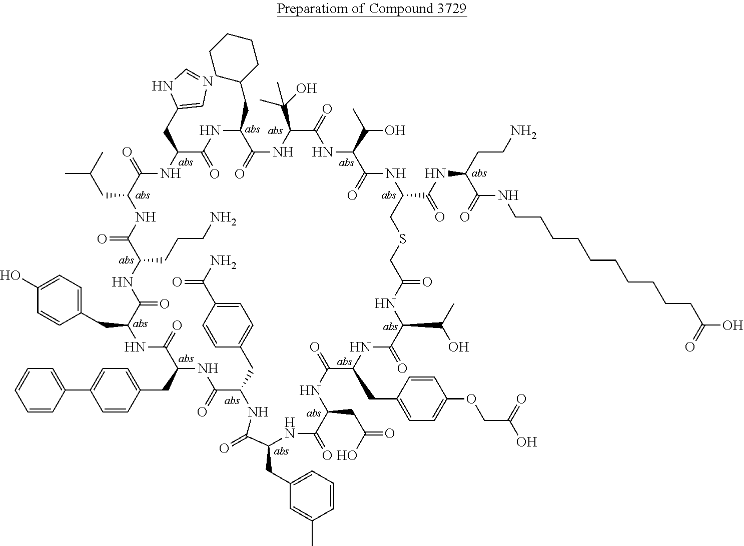 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 3729 was prepared on a 30 μmol scale. The yield of the product was 5.7 mg, and its estimated purity by LCMS analysis was 87%. Analysis condition B: Retention time=1.65 min; ESI-MS(+) m/z [M+2H]²⁺: 1177.2.

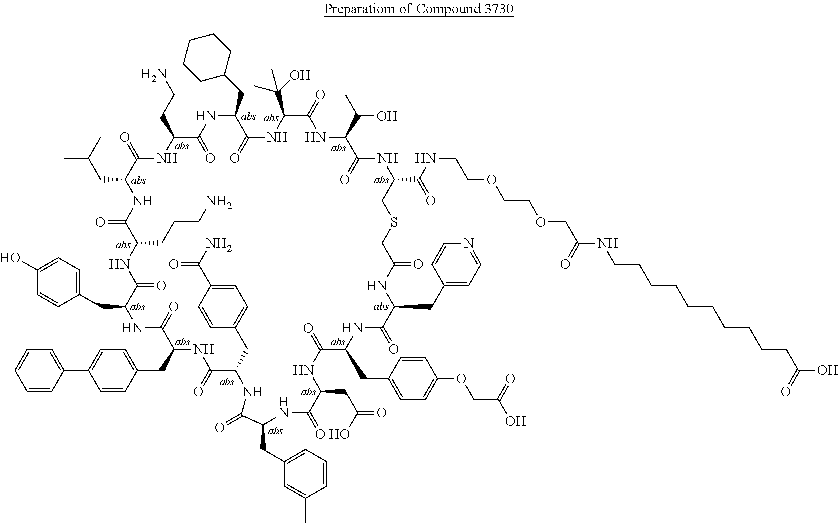 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)NCCOCCOCC(=O)NCCCCCCCCCCC(=O)O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 3730 was prepared on a 30 μmol scale. The yield of the product was 10.8 mg, and its estimated purity by LCMS analysis was 96.1%. Analysis condition B: Retention time=1.76 min; ESI-MS(+) m/z [M+2H]²⁺: 1204.9.

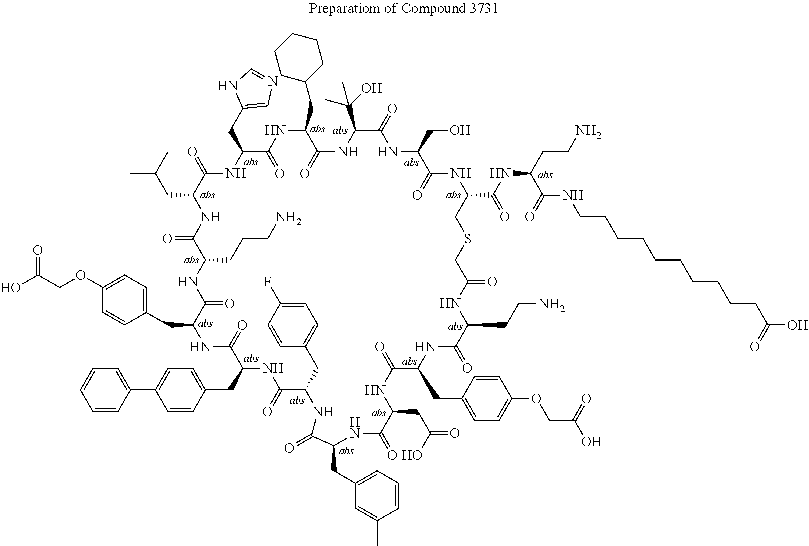 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(F)cc3)NC2=O)c1]

Compound 3731 was prepared on a 50 μmol scale. The yield of the product was 14.5 mg, and its estimated purity by LCMS analysis was 97.5%. Analysis condition B: Retention time=1.72 min; ESI-MS(+) m/z [M+3H]³⁺: 791.2.

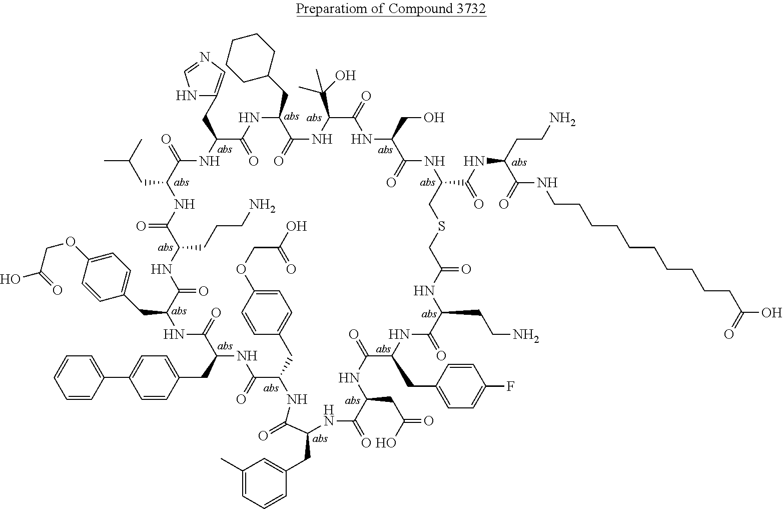 [SMILES: Cc1cccc(C[C@@H]2NC(=O)[C@H](CC(=O)O)NC(=O)[C@H](Cc3ccc(F)cc3)NC(=O)[C@H](CCN)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3732 was prepared on a 50 μmol scale. The yield of the product was 2.6 mg, and its estimated purity by LCMS analysis was 95.8%. Analysis condition A: Retention time-1.67 min; ESI-MS(+) m/z [M+2H]²⁺: 1186.1.

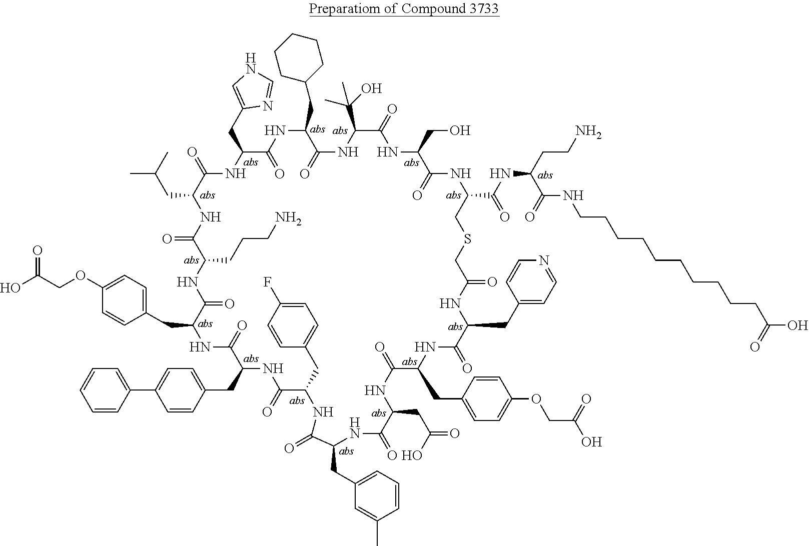 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(F)cc3)NC2=O)c1]

Compound 3733 was prepared on a 50 μmol scale. The yield of the product was 21.3 mg, and its estimated purity by LCMS analysis was 98.6%. Analysis condition A: Retention time=1.64 min; ESI-MS(+) m/z [M+2H]²⁺: 1210.

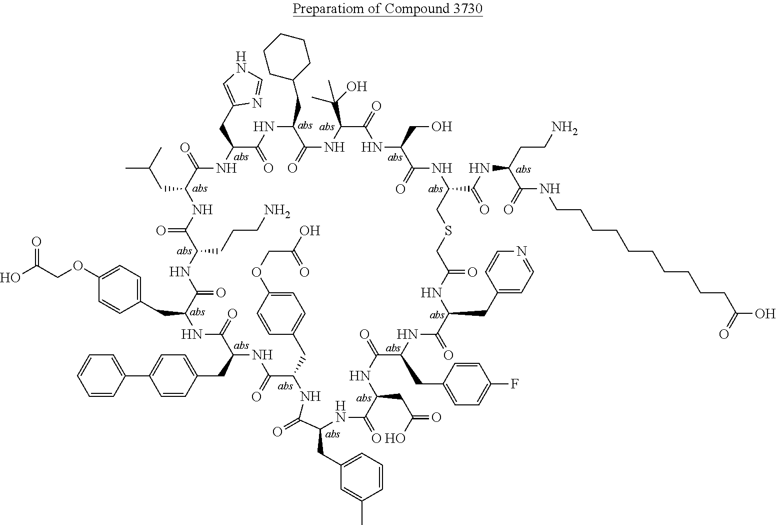 [SMILES: Cc1cccc(C[C@@H]2NC(=O)[C@H](CC(=O)O)NC(=O)[C@H](Cc3ccc(F)cc3)NC(=O)[C@H](Cc3ccncc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3c[nH]cn3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3734 was prepared on a 50 μmol scale. The yield of the product was 3.9 mg, and its estimated purity by LCMS analysis was 98.2%. Analysis condition A: Retention time=1.76 min; ESI-MS(+) m/z [M+2H]²⁺: 1210.

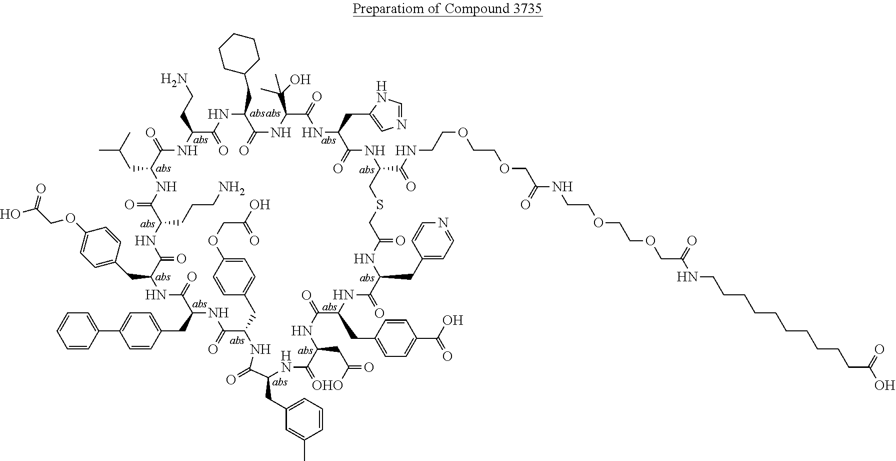 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)NCCOCCOCC(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3735 was prepared on a 50 μmol scale. The yield of the product was 24.8 mg, and its estimated purity by LCMS analysis was 84.9%. Analysis condition B: Retention time=1.6 min; ESI-MS(+) m/z [M+2H]²⁺: 1325.

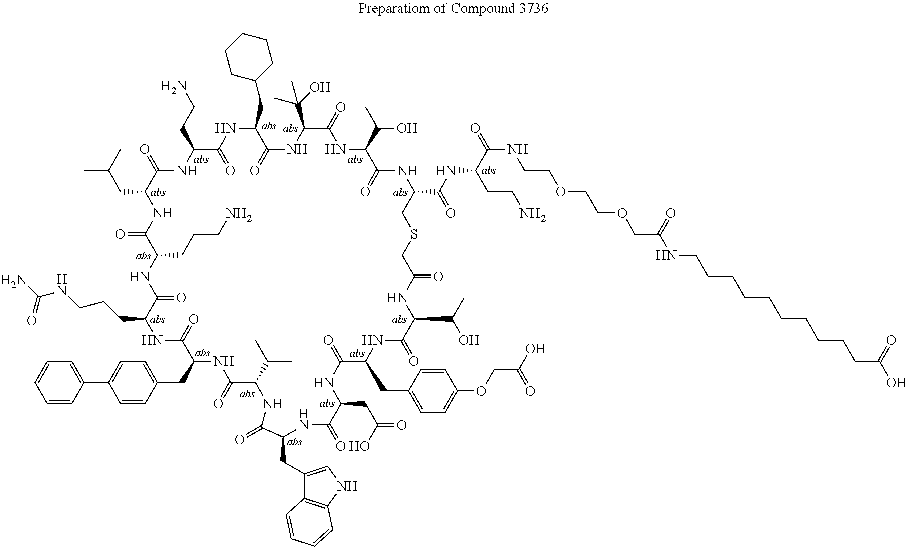 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](C(C)O)NC(=O)[C@H](C(C)(C)O)NC(=O)[C@H](CC2CCCCC2)NC(=O)[C@H](CCN)NC1=O]

Compound 3736 was prepared on a 50 μmol scale. The yield of the product was 16.5 mg, and its estimated purity by LCMS analysis was 100%. Analysis condition A: Retention time=1.62 min; ESI-MS(+) m/z [M+2H]²⁺: 1195.

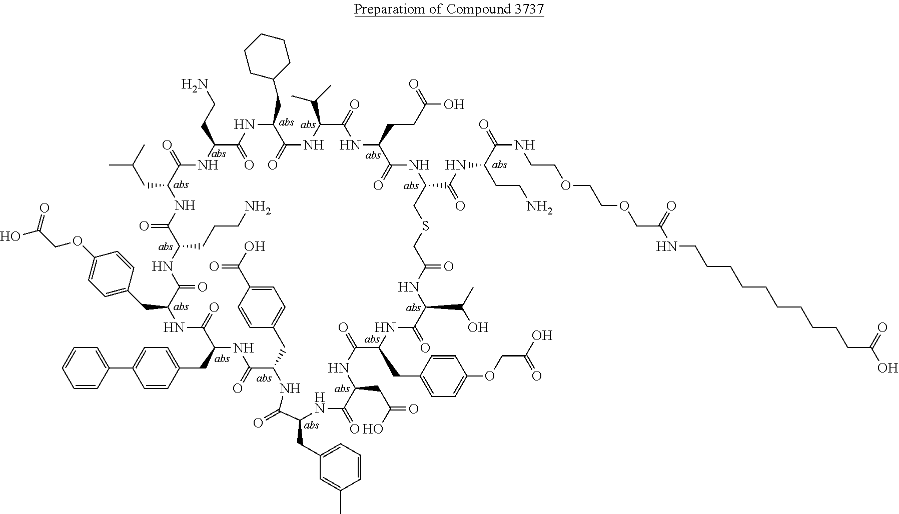 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3737 was prepared on a 50 μmol scale. The yield of the product was 7.5 mg, and its estimated purity by LCMS analysis was 97.8%. Analysis condition A: Retention time-1.41 min; ESI-MS(+) m/z [M+2H]²⁺: 1267.

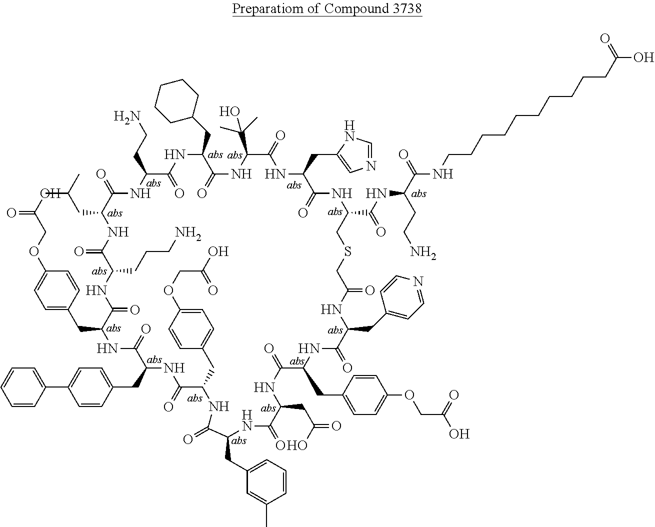 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@H](CCN)C(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3738 was prepared on a 50 μmol scale. The yield of the product was 0.7 mg, and its estimated purity by LCMS analysis was 100%. Analysis condition A: Retention time=1.41 min; ESI-MS(+) m/z [M+2H]²⁺: 1245.1.

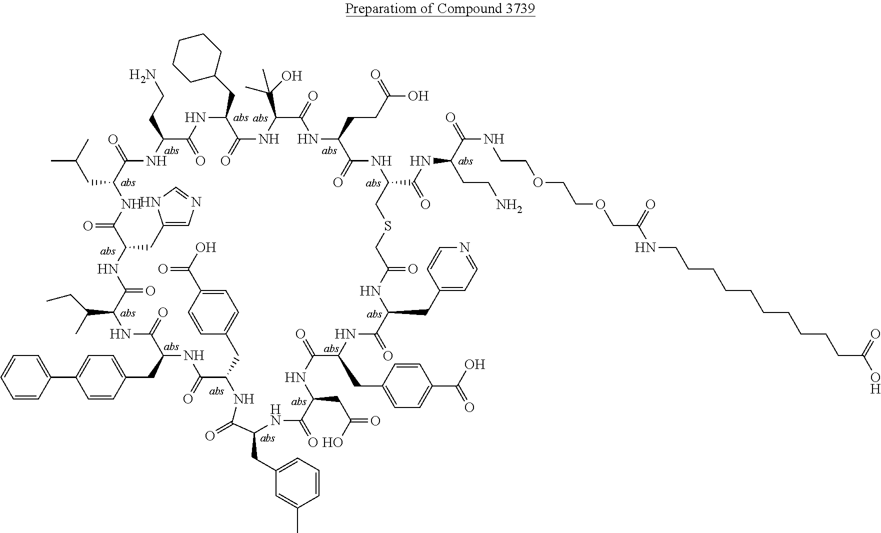 [SMILES: CCC(C)[C@@H]1NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](Cc2cnc[nH]2)NC1=O]

Compound 3739 was prepared on a 50 μmol scale. The yield of the product was 39.9 mg, and its estimated purity by LCMS analysis was 96.5%. Analysis condition B: Retention time=1.68 min; ESI-MS(+) m/z [M+3H]³⁺: 828.

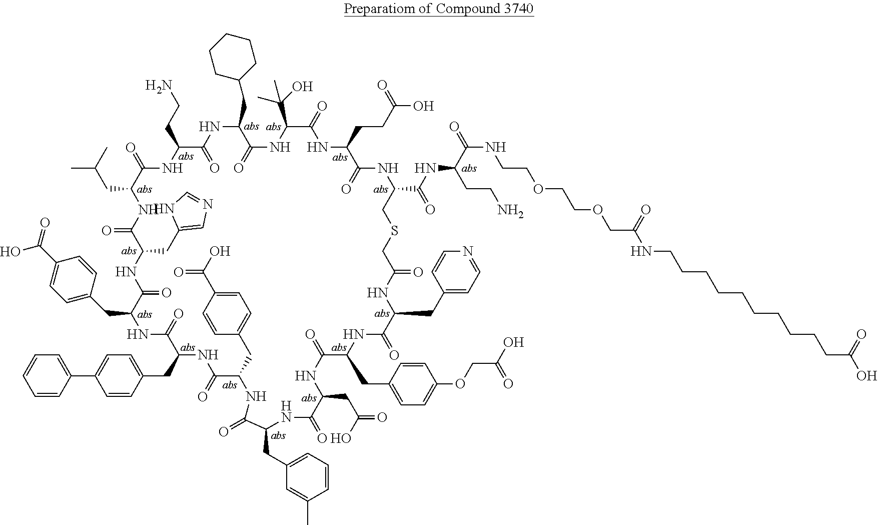 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](Cc3cnc[nH]3)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3740 was prepared on a 50 μmol scale. The yield of the product was 90.6 mg, and its estimated purity by LCMS analysis was 96.7%. Analysis condition B: Retention time=1.59 min; ESI-MS(+) m/z [M+2H]²⁺: 1295.2.

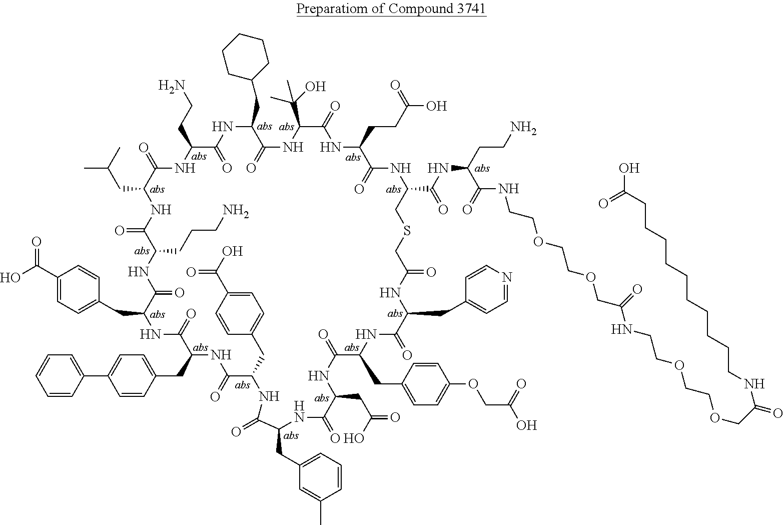 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3741 was prepared on a 50 μmol scale. The yield of the product was 63.3 mg, and its estimated purity by LCMS analysis was 93.5%. Analysis condition B: Retention time=1.59 min; ESI-MS(+) m/z [M+2H]²⁺: 1356.1.

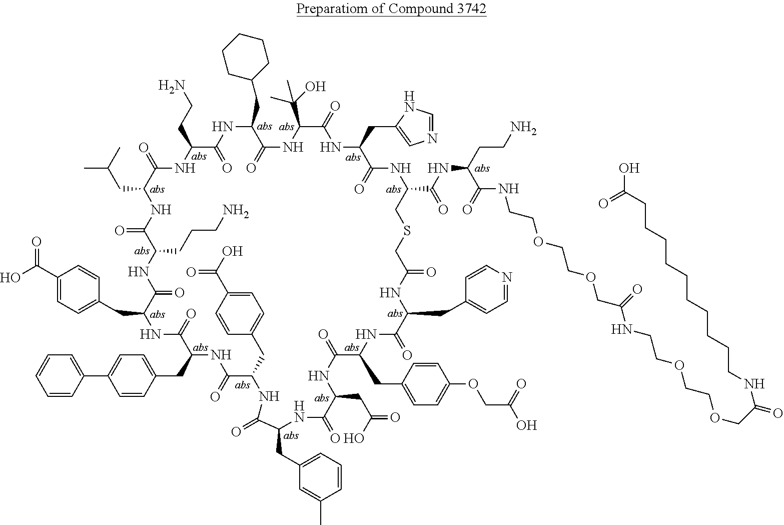 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3742 was prepared on a 50 μmol scale. The yield of the product was 81.9 mg, and its estimated purity by LCMS analysis was 91.9%. Analysis condition A: Retention time=1.38 min; ESI-MS(+) m/z [M+2H]²⁺: 1360.1.

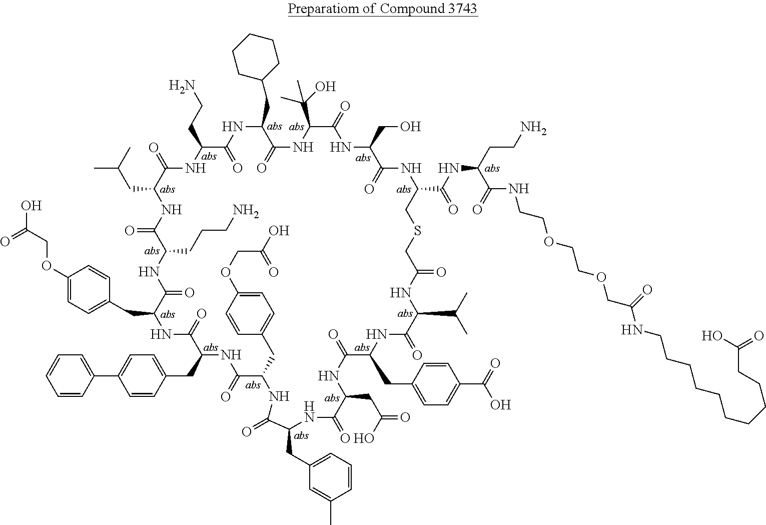 [SMILES: Cc1cccc(C[C@@H]2NC(=O)[C@H](CC(=O)O)NC(=O)[C@H](Cc3ccc(C(=O)O)cc3)NC(=O)[C@H](C(C)C)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3743 was prepared on a 50 μmol scale. The yield of the product was 14.2 mg, and its estimated purity by LCMS analysis was 95.2%. Analysis condition B: Retention time=1.76 min; ESI-MS(+) m/z [M+2H]²⁺: 1254.1.

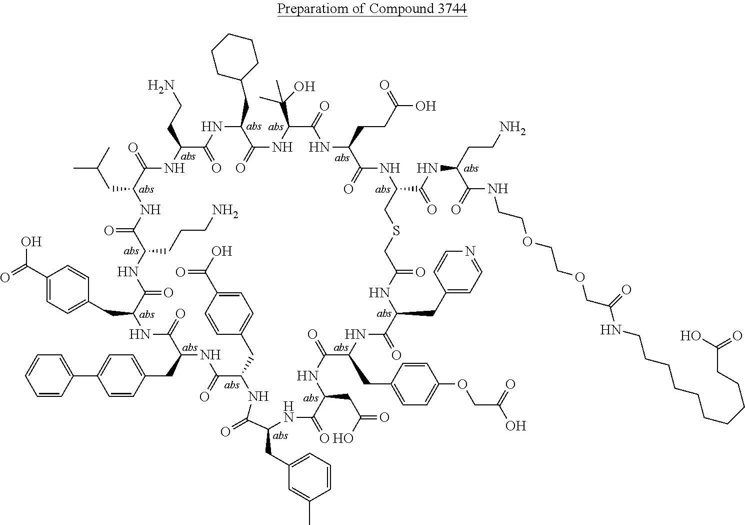 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3744 was prepared on a 50 μmol scale. The yield of the product was 53 mg, and its estimated purity by LCMS analysis was 95.1%. Analysis condition B: Retention time=1.66 min; ESI-MS(+) m/z [M+3H]³⁺: 856.2.

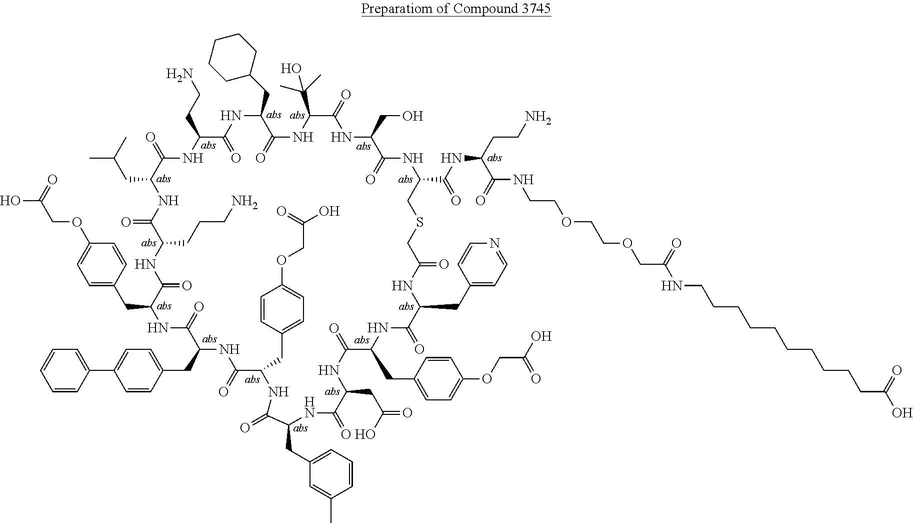 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3745 was prepared on a 50 μmol scale. The yield of the product was 38.8 mg, and its estimated purity by LCMS analysis was 100%. Analysis condition A: Retention time=1.44 min; ESI-MS(+) m/z [M+2H]²⁺: 1299.4.

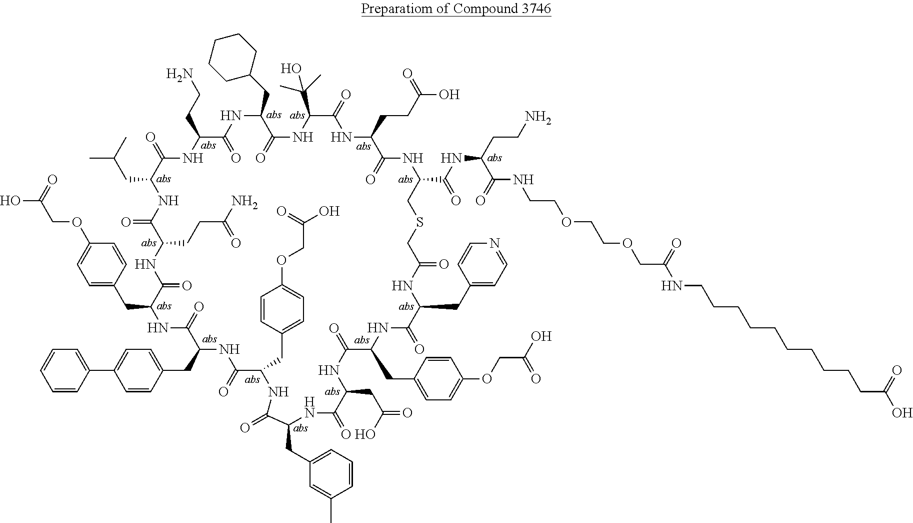 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3746 was prepared on a 50 μmol scale. The yield of the product was 25.5 mg, and its estimated purity by LCMS analysis was 96.8%. Analysis condition B: Retention time-1.73 min; ESI-MS(+) m/z [M+3H]³⁺: 881.1.

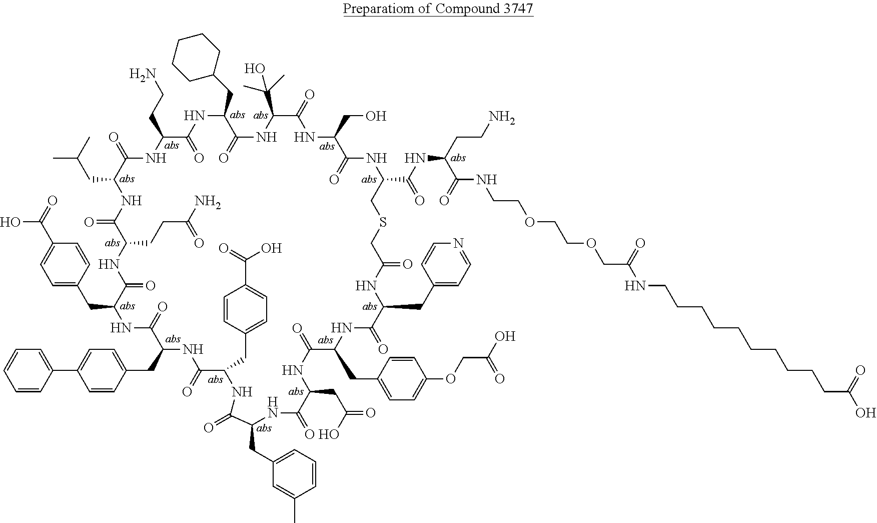 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3747 was prepared on a 50 μmol scale. The yield of the product was 25.6 mg, and its estimated purity by LCMS analysis was 99.5%. Analysis condition B: Retention time=1.63 min; ESI-MS(+) m/z [M+3H]³⁺: 847.1.

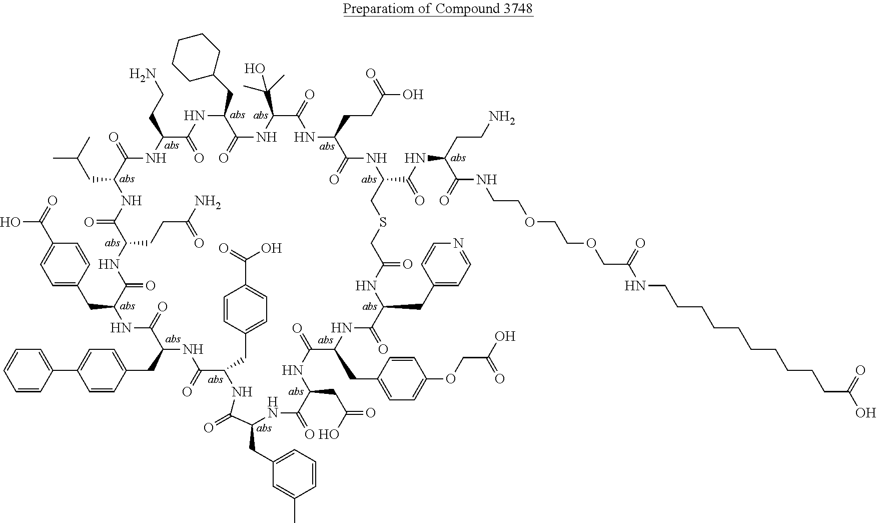 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3748 was prepared on a 50 μmol scale. The yield of the product was 57.2 mg, and its estimated purity by LCMS analysis was 95.7%. Analysis condition B: Retention time=1.72 min; ESI-MS(+) m/z [M+3H]³⁺: 861.

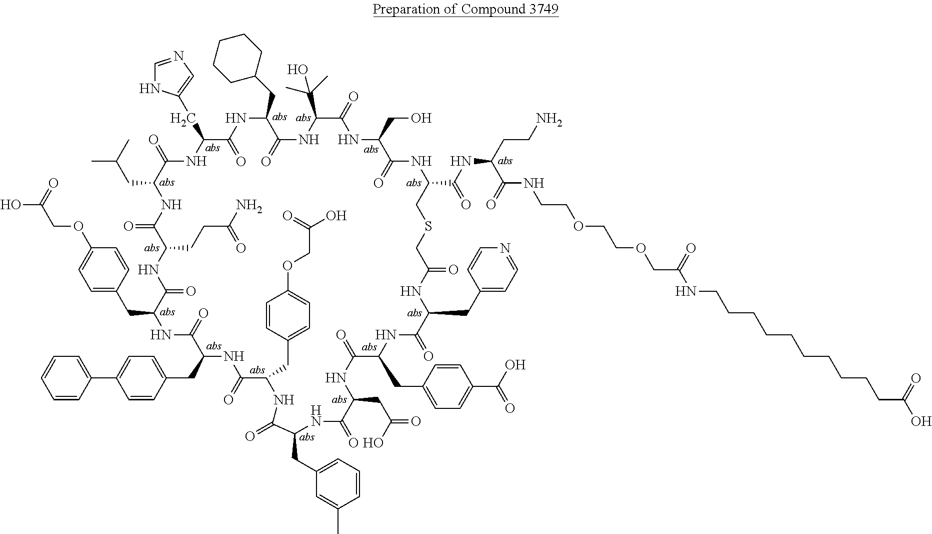 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3749 was prepared on a 50 μmol scale. The yield of the product was 16.2 mg, and its estimated purity by LCMS analysis was 96%. Analysis condition B: Retention time=1.77 min; ESI-MS(+) m/z [M+3H]³⁺: 869.3.

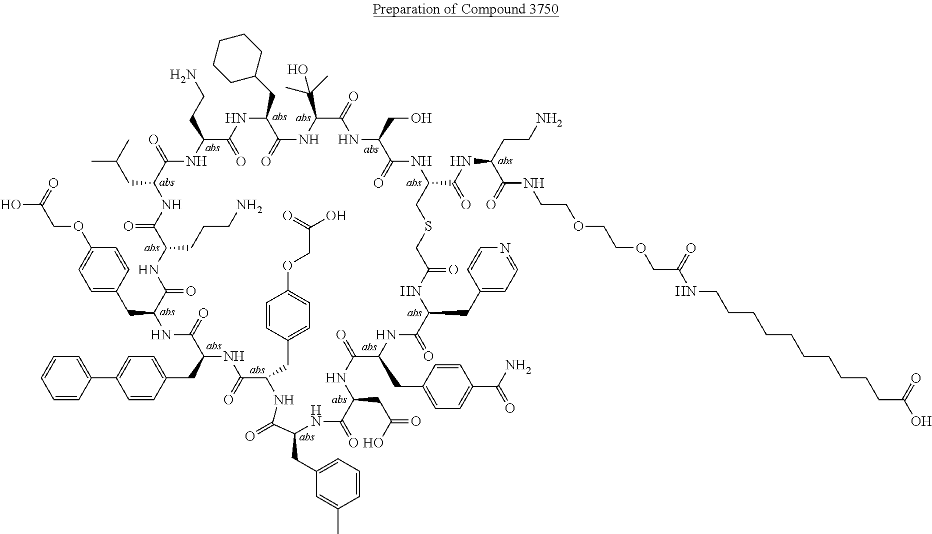 [SMILES: Cc1cccc(C[C@@H]2NC(=O)[C@H](CC(=O)O)NC(=O)[C@H](Cc3ccc(C(N)=O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3750 was prepared on a 50 μmol scale. The yield of the product was 5.6 mg, and its estimated purity by LCMS analysis was 95%. Analysis condition B: Retention time=1.59 min; ESI-MS(+) m/z [M+2H]²⁺: 1277.

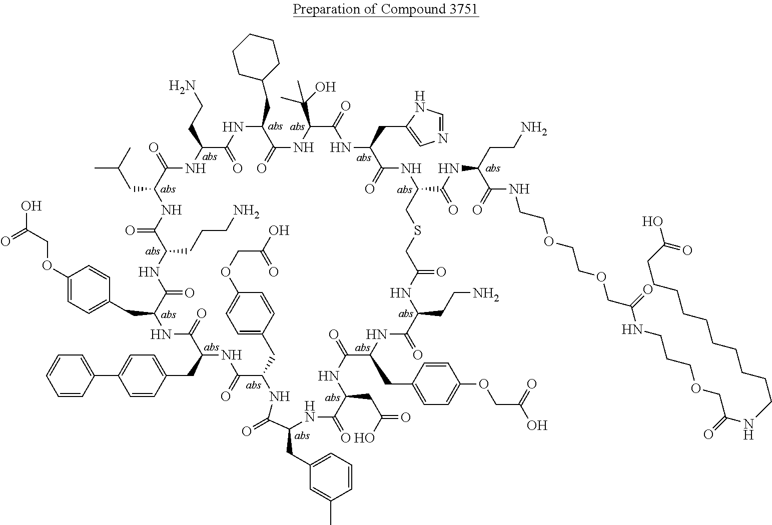 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3751 was prepared on a 50 μmol scale. The yield of the product was 13 mg, and its estimated purity by LCMS analysis was 100%. Analysis condition A: Retention time=1.59 min; ESI-MS(+) m/z [M+3H]³⁺: 911.1.

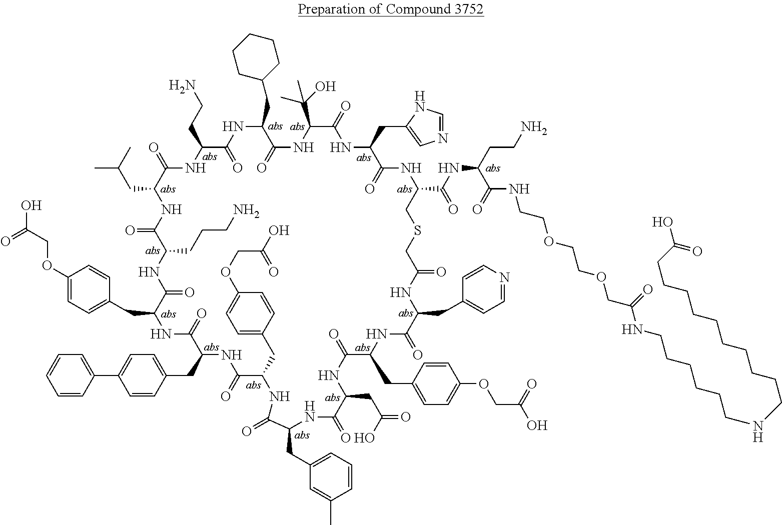 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCNCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3752 was prepared on a 50 μmol scale. The yield of the product was 38.9 mg, and its estimated purity by LCMS analysis was 92.7%. Analysis condition A: Retention time=1.45 min; ESI-MS(+) m/z [M+2H]²⁺: 1389.9.

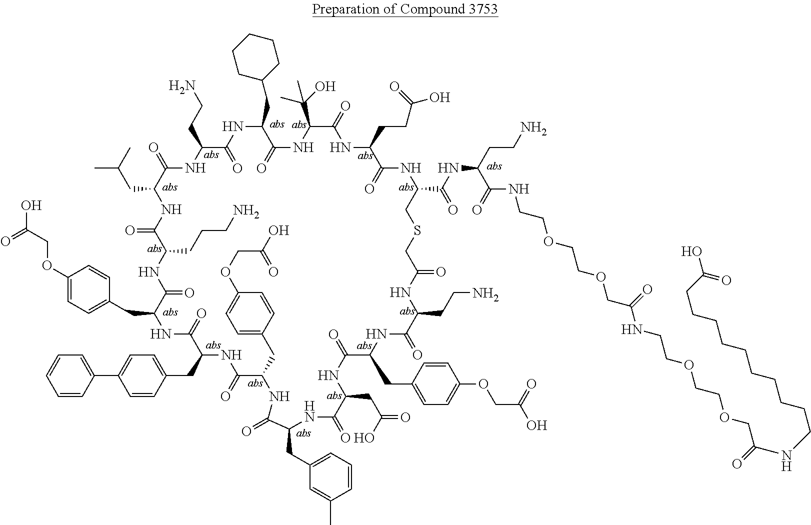 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3753 was prepared on a 50 μmol scale. The yield of the product was 30.8 mg, and its estimated purity by LCMS analysis was 100%. Analysis condition B: Retention time-1.69 min; ESI-MS(+) m/z [M+2H]²⁺: 1362.2.

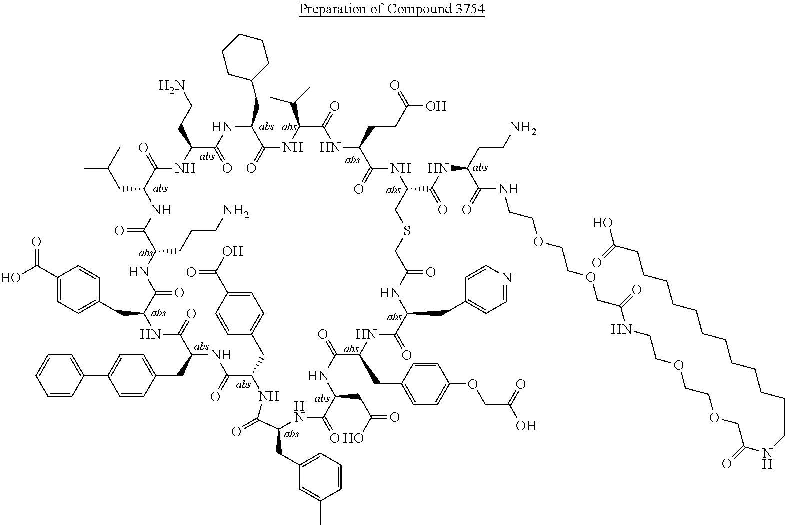 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3754 was prepared on a 50 μmol scale. The yield of the product was 5.6 mg, and its estimated purity by LCMS analysis was 98.7%. Analysis condition B: Retention time=1.68 min; ESI-MS(+) m/z [M+2H]²⁺: 1362.1.

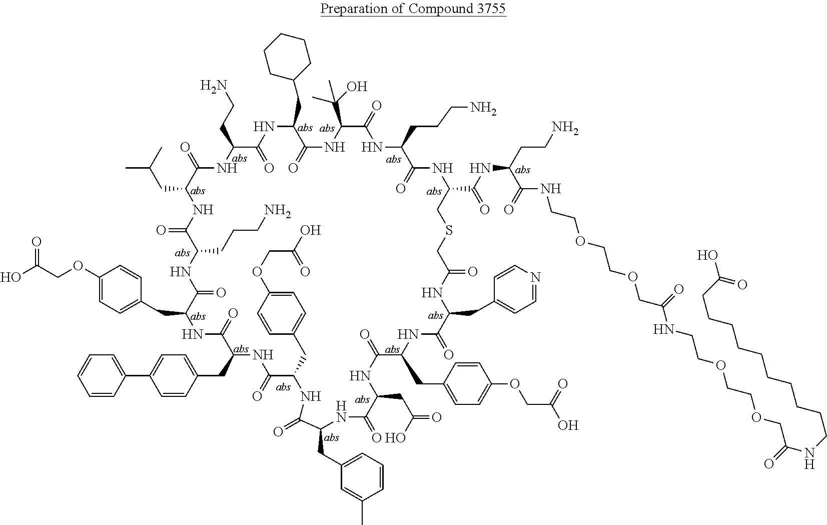 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCCN)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3755 was prepared on a 50 μmol scale. The yield of the product was 4.1 mg, and its estimated purity by LCMS analysis was 96.9%. Analysis condition A: Retention time=1.49 min; ESI-MS(+) m/z [M+2H]²⁺: 1378.9.

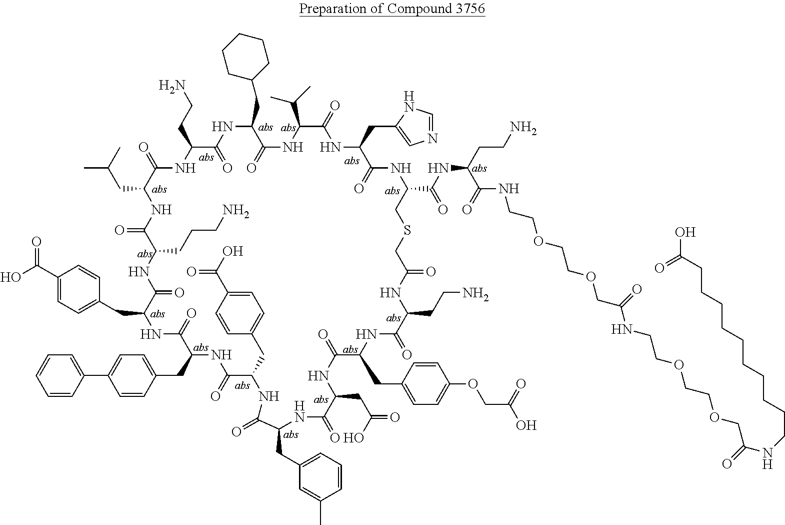 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3756 was prepared on a 50 μmol scale. The yield of the product was 8.4 mg, and its estimated purity by LCMS analysis was 96.4%. Analysis condition B: Retention time-1.55 min; ESI-MS(+) m/z [M+2H]²⁺: 1336.3.

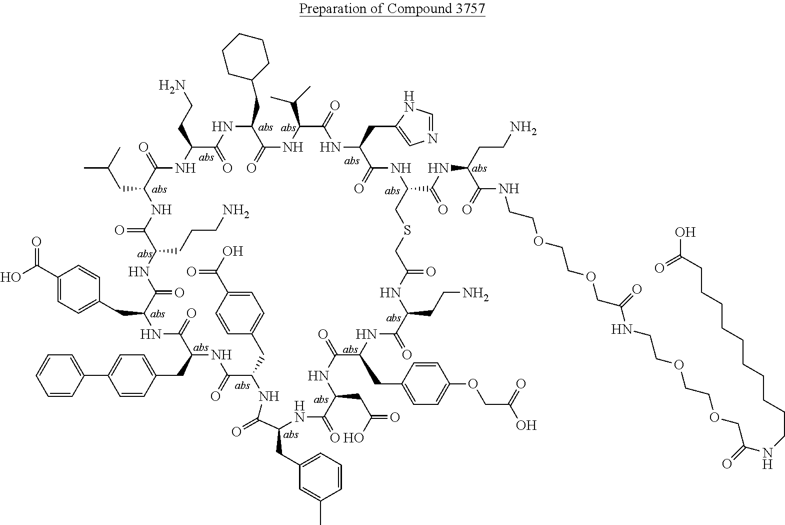 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3757 was prepared on a 50 μmol scale. The yield of the product was 15.6 mg, and its estimated purity by LCMS analysis was 96%. Analysis condition B: Retention time=1.56 min; ESI-MS(+) m/z [M+2H]²⁺: 1328.

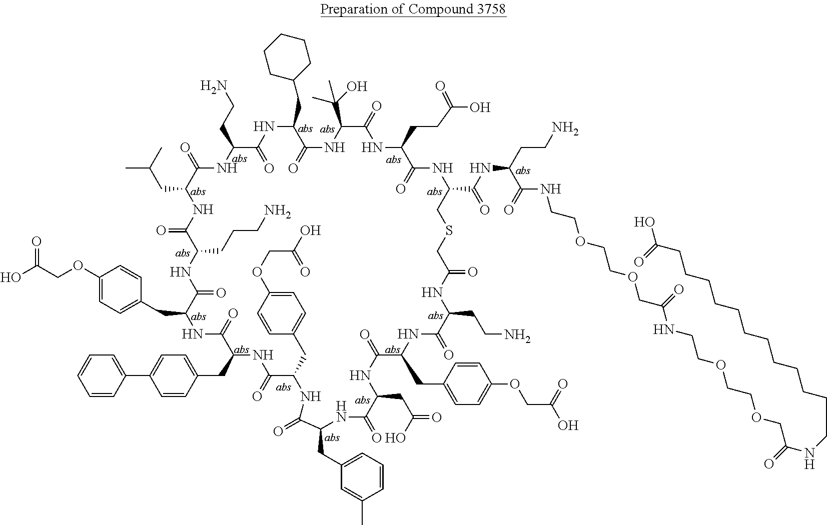 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3758 was prepared on a 50 μmol scale. The yield of the product was 25.5 mg, and its estimated purity by LCMS analysis was 96%. Analysis condition B: Retention time=1.68 min; ESI-MS(+) m/z [M+2H]²⁺: 1375.8.

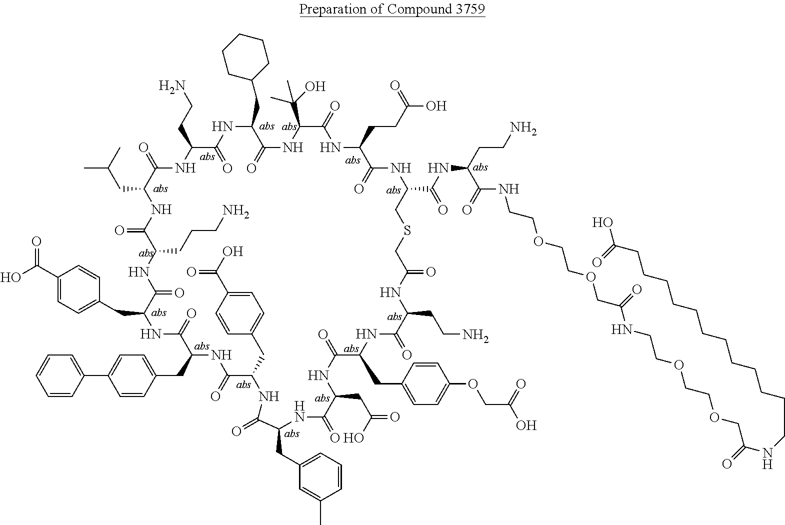 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3759 was prepared on a 50 μmol scale. The yield of the product was 27.5 mg, and its estimated purity by LCMS analysis was 100%. Analysis condition A: Retention time=1.57 min; ESI-MS(+) m/z [M+2H]²⁺: 1346.3.

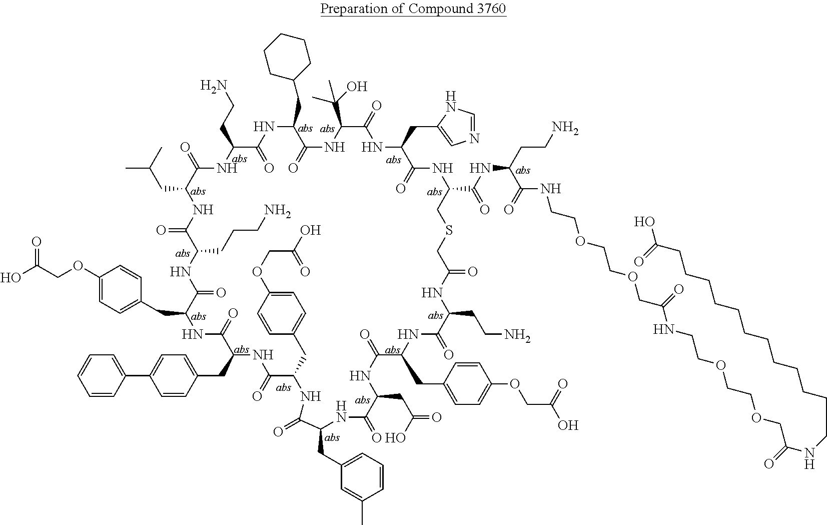 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3760 was prepared on a 50 μmol scale. The yield of the product was 33.3 mg, and its estimated purity by LCMS analysis was 95.2%. Analysis condition B: Retention time=1.73 min; ESI-MS(+) m/z [M+2H]²⁺: 920.1.

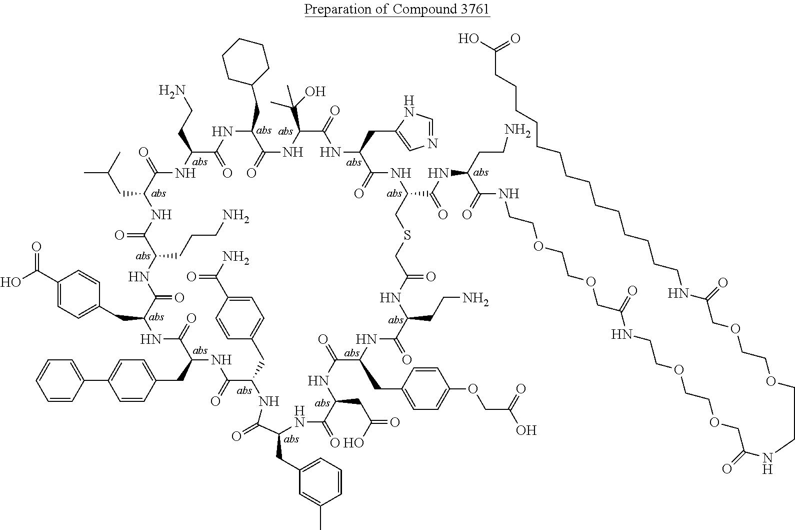 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCOCCOCC(=O)NCCCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(N)=O)cc3)NC2=O)c1]

Compound 3761 was prepared on a 50 μmol scale. The yield of the product was 51.2 mg, and its estimated purity by LCMS analysis was 100%. Analysis condition A: Retention time=1.64 min; ESI-MS(+) m/z [M+3H]³⁺: 958.1.

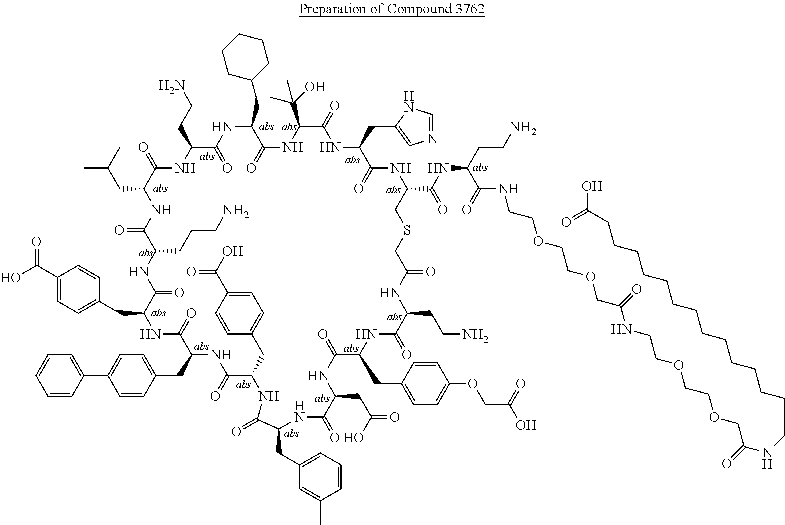 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3762 was prepared on a 50 μmol scale. The yield of the product was 50.1 mg, and its estimated purity by LCMS analysis was 89.9%. Analysis condition A: Retention time=1.66 min; ESI-MS(+) m/z [M+2H]²⁺: 1364.

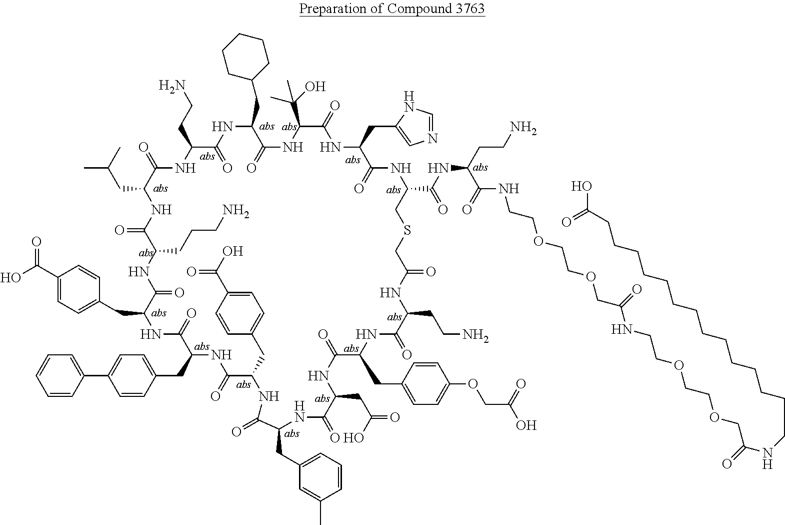 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3763 was prepared on a 50 μmol scale. The yield of the product was 16.4 mg, and its estimated purity by LCMS analysis was 97.5%. Analysis condition B: Retention time=1.71 min; ESI-MS(+) m/z [M+2H]²⁺: 1350.

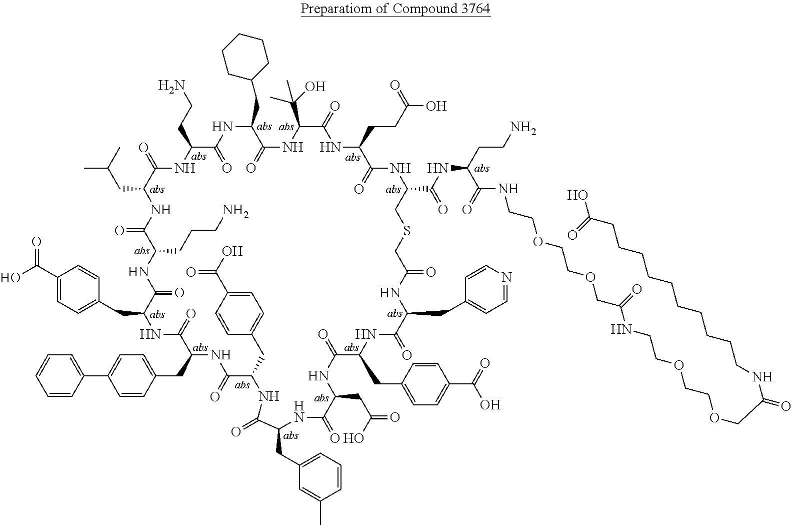 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3764 was prepared on a 50 μmol scale. The yield of the product was 31 mg, and its estimated purity by LCMS analysis was 99.3%. Analysis condition B: Retention time=1.67 min; ESI-MS(+) m/z [M+2H]²⁺: 1341.3.

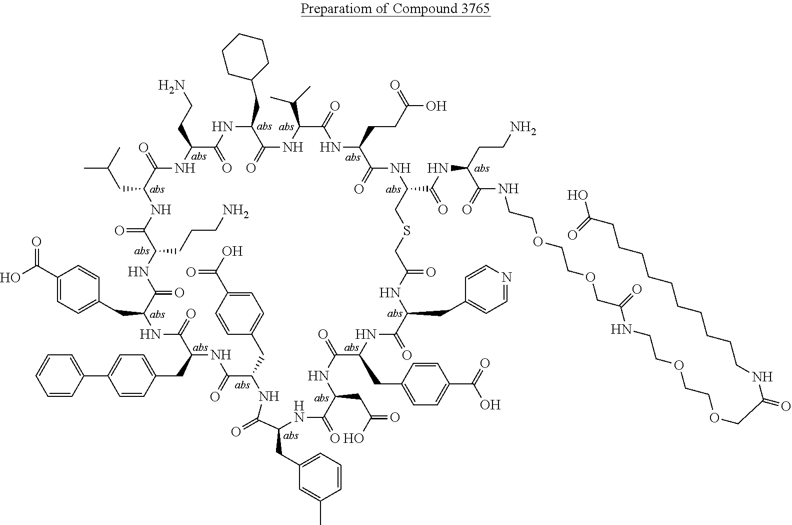 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3765 was prepared on a 50 μmol scale. The yield of the product was 16.6 mg, and its estimated purity by LCMS analysis was 95.7%. Analysis condition B: Retention time=1.66 min; ESI-MS(+) m/z [M+2H]²⁺: 1333.3.

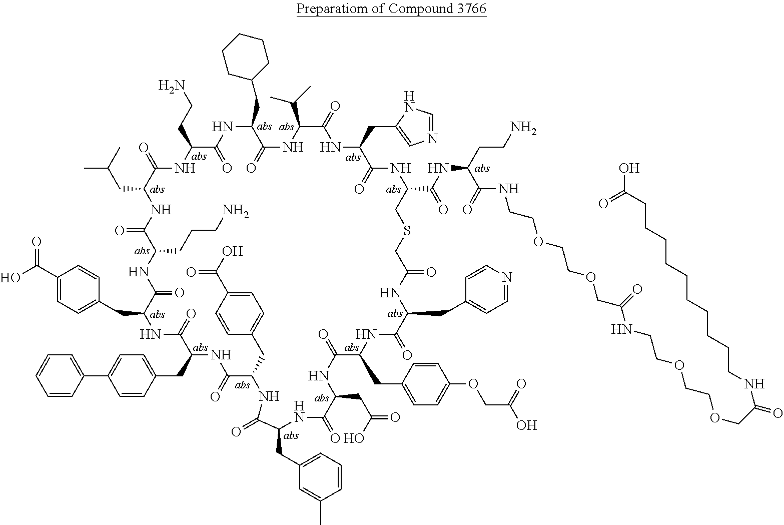 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3766 was prepared on a 50 μmol scale. The yield of the product was 10.3 mg, and its estimated purity by LCMS analysis was 96.4%. Analysis condition B: Retention time=1.56 min; ESI-MS(+) m/z [M+2H]²⁺: 1352.1.

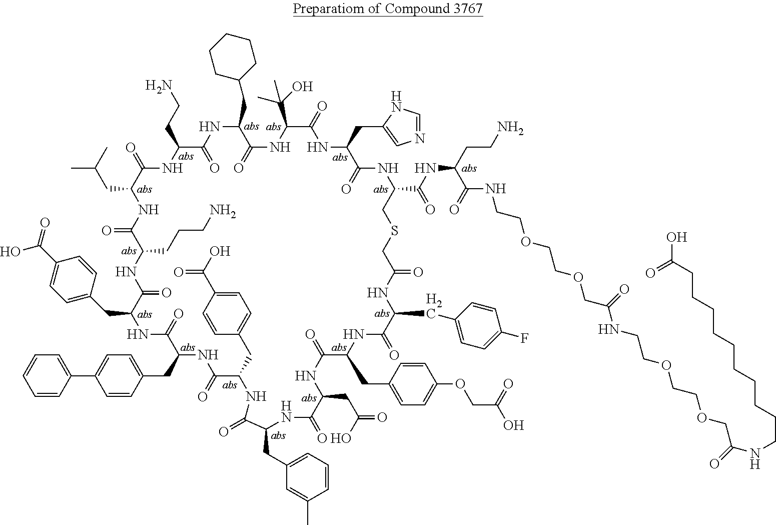 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3767 was prepared on a 50 μmol scale. The yield of the product was 7.3 mg, and its estimated purity by LCMS analysis was 100%. Analysis condition A: Retention time=1.61 min; ESI-MS(+) m/z [M+2H]²⁺: 1368.9.

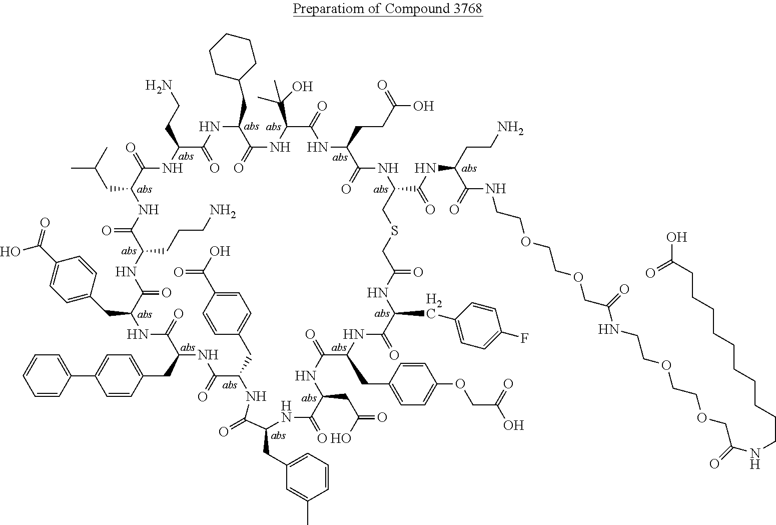 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3768 was prepared on a 50 μmol scale. The yield of the product was 33.4 mg, and its estimated purity by LCMS analysis was 98.4%. Analysis condition A: Retention time=1.41 min; ESI-MS(+) m/z [M+2H]²⁺: 1364.5.

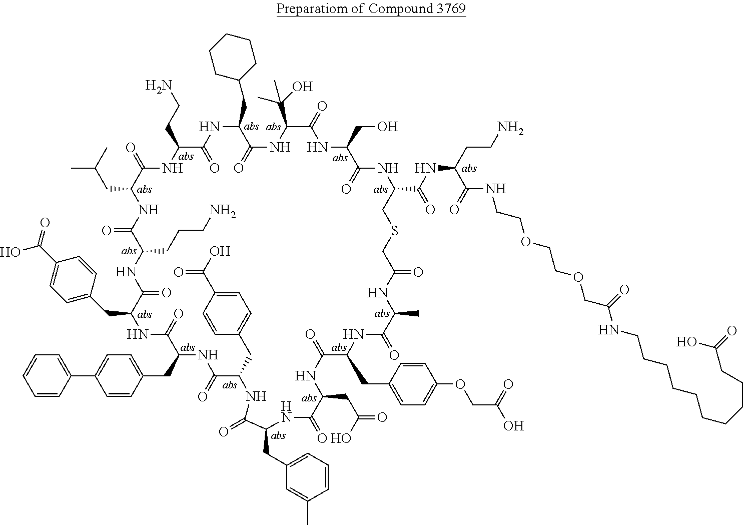 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3769 was prepared on a 50 μmol scale. The yield of the product was 40 mg, and its estimated purity by LCMS analysis was 93%. Analysis condition B: Retention time=1.74 min; ESI-MS(+) m/z [M+2H]²⁺: 1224.2.

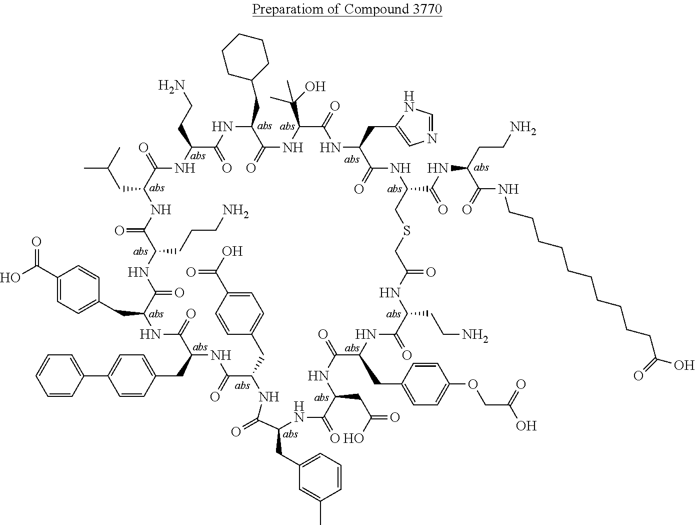 [SMILES: Cc1cccc(C[C@@H]2NC(=O)[C@H](CC(=O)O)NC(=O)[C@H](Cc3ccc(OCC(=O)O)cc3)NC(=O)[C@@H](CCN)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3770 was prepared on a 50 μmol scale. The yield of the product was 10.5 mg, and its estimated purity by LCMS analysis was 92.8%. Analysis condition B: Retention time=1.5 min; ESI-MS(+) m/z [M+2H]²⁺: 1191.

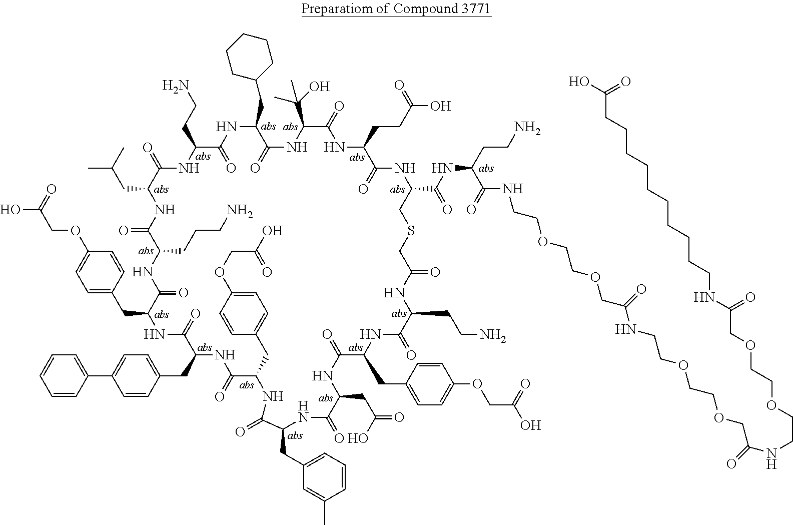 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCOCCOCC(=O)NCCCCCCCCCCC(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3771 was prepared on a 50 μmol scale. The yield of the product was 36.9 mg, and its estimated purity by LCMS analysis was 97.5%. Analysis condition A: Retention time=1.39 min; ESI-MS(+) m/z [M+2H]²⁺: 1435.4.

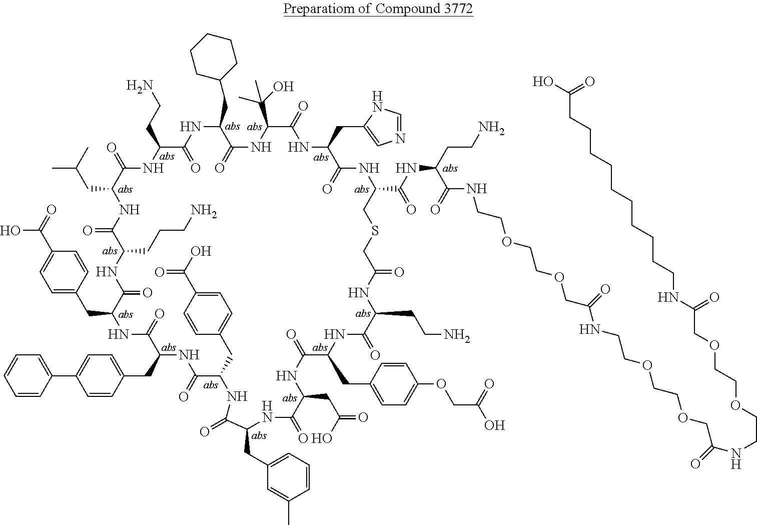 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3772 was prepared on a 50 μmol scale. The yield of the product was 25.4 mg, and its estimated purity by LCMS analysis was 95.2%. Analysis condition A: Retention time=1.61 min; ESI-MS(+) m/z [M+2H]²⁺: 939.2.

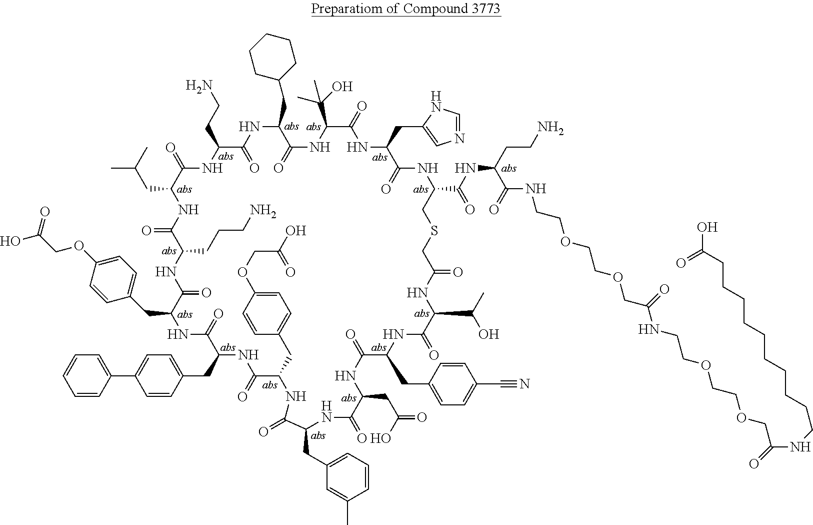 [SMILES: Cc1cccc(C[C@@H]2NC(=O)[C@H](CC(=O)O)NC(=O)[C@H](Cc3ccc(C#N)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3773 was prepared on a 50 μmol scale. The yield of the product was 24.9 mg, and its estimated purity by LCMS analysis was 97.5%. Analysis condition B: Retention time=1.73 min; ESI-MS(+) m/z [M+2H]²⁺: 1342.3.

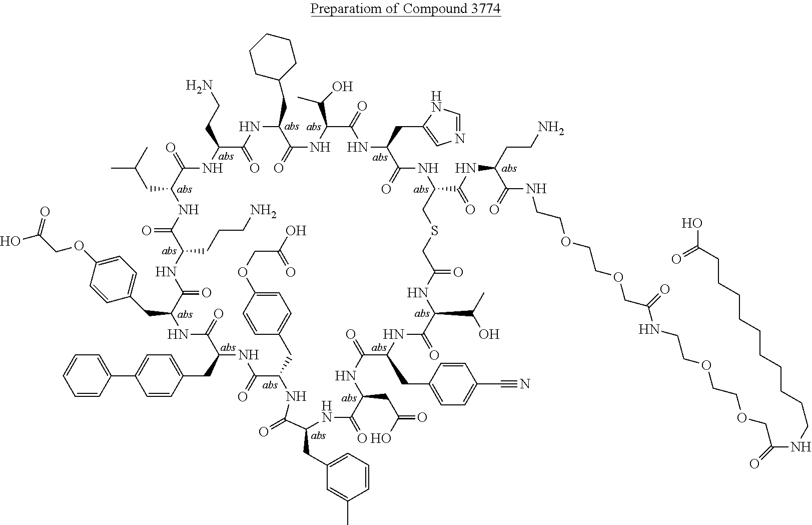 [SMILES: Cc1cccc(C[C@@H]2NC(=O)[C@H](CC(=O)O)NC(=O)[C@H](Cc3ccc(C#N)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3774 was prepared on a 50 μmol scale. The yield of the product was 39.8 mg, and its estimated purity by LCMS analysis was 97.8%. Analysis condition B: Retention time=1.67 min; ESI-MS(+) m/z [M+2H]²⁺: 1335.2.

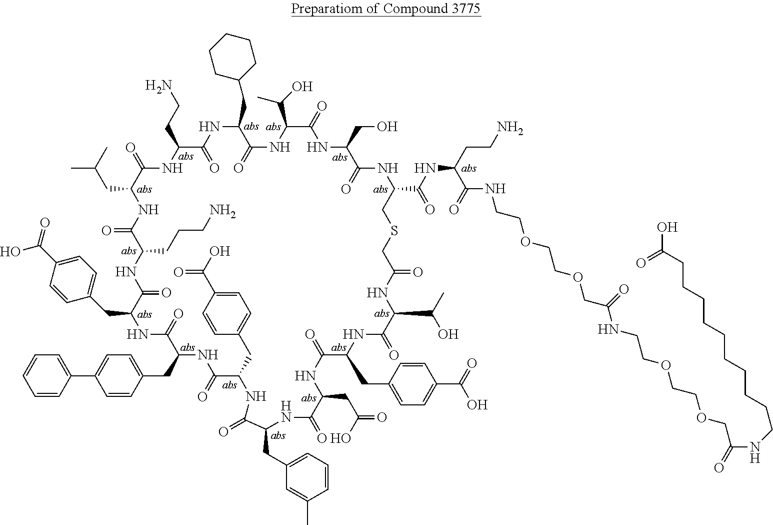 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3775 was prepared on a 50 μmol scale. The yield of the product was 4.9 mg, and its estimated purity by LCMS analysis was 95.2%. Analysis condition A: Retention time=1.41 min; ESI-MS(+) m/z [M+2H]²⁺: 1289.4.

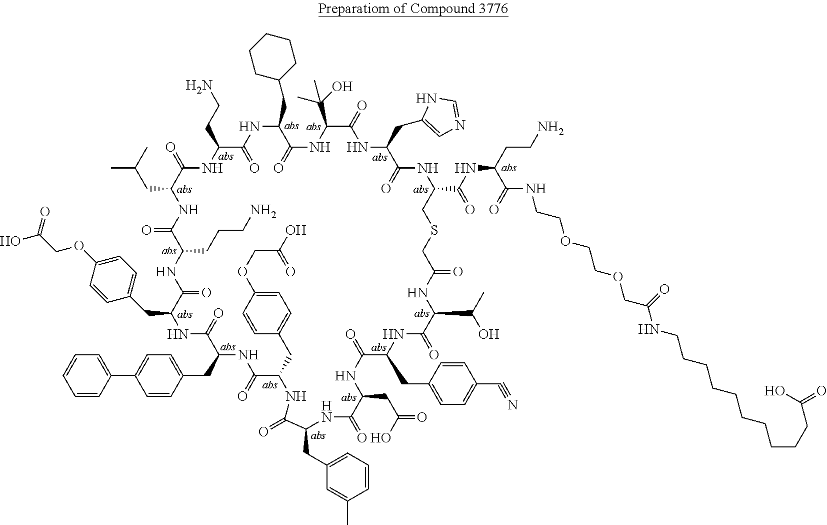 [SMILES: Cc1cccc(C[C@@H]2NC(=O)[C@H](CC(=O)O)NC(=O)[C@H](Cc3ccc(C#N)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3776 was prepared on a 50 μmol scale. The yield of the product was 12.9 mg, and its estimated purity by LCMS analysis was 100%. Analysis condition A: Retention time=1.72 min; ESI-MS(+) m/z [M+2H]²⁺: 1270.

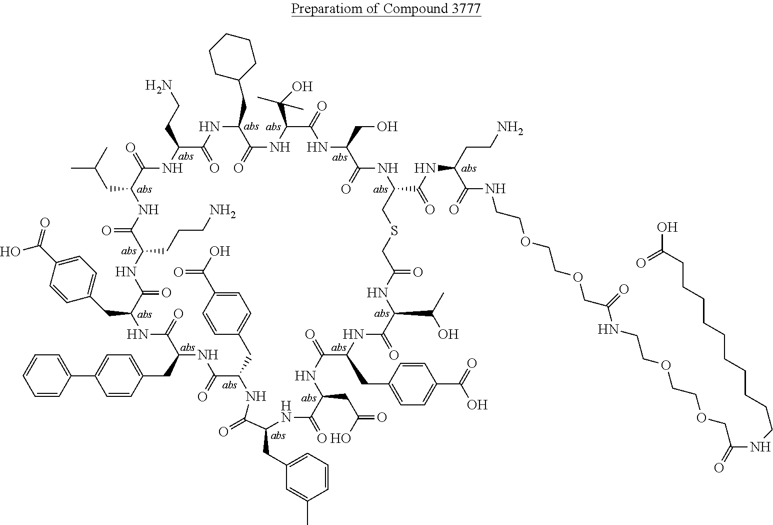 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3777 was prepared on a 50 μmol scale. The yield of the product was 11.4 mg, and its estimated purity by LCMS analysis was 96%. Analysis condition A: Retention time=1.46 min; ESI-MS(+) m/z [M+2H]²⁺: 1296.2.

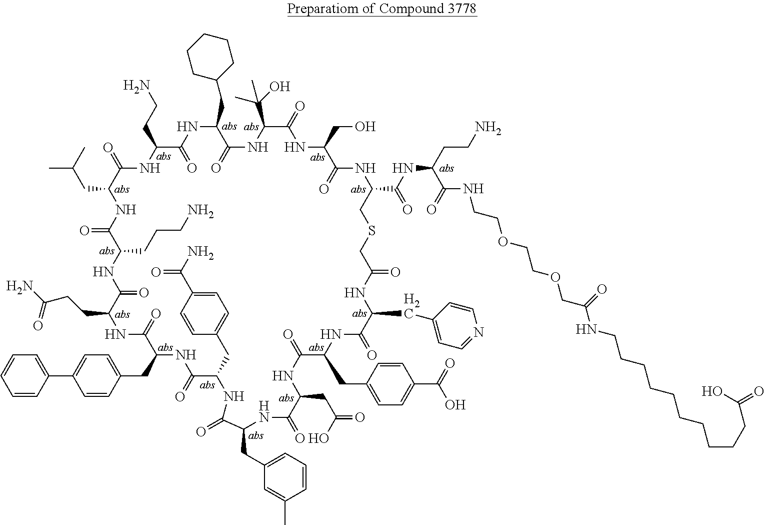 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](CCC(N)=O)NC(=O)[C@H](Cc3ccc(-c4ccccc4)cc3)NC(=O)[C@H](Cc3ccc(C(N)=O)cc3)NC2=O)c1]

Compound 3778 was prepared on a 50 μmol scale. The yield of the product was 35 mg, and its estimated purity by LCMS analysis was 95.5%. Analysis condition B: Retention time=1.86 min; ESI-MS(+) m/z [M+2H]²⁺: 1215.3.

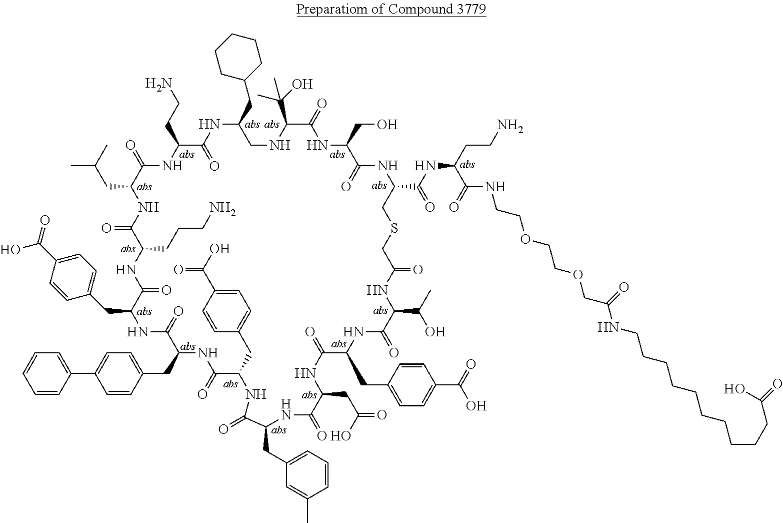 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3779 was prepared on a 50 μmol scale. The yield of the product was 6.8 mg, and its estimated purity by LCMS analysis was 100%. Analysis condition A: Retention time-1.41 min; ESI-MS(+) m/z [M+2H]²⁺: 1224.1.

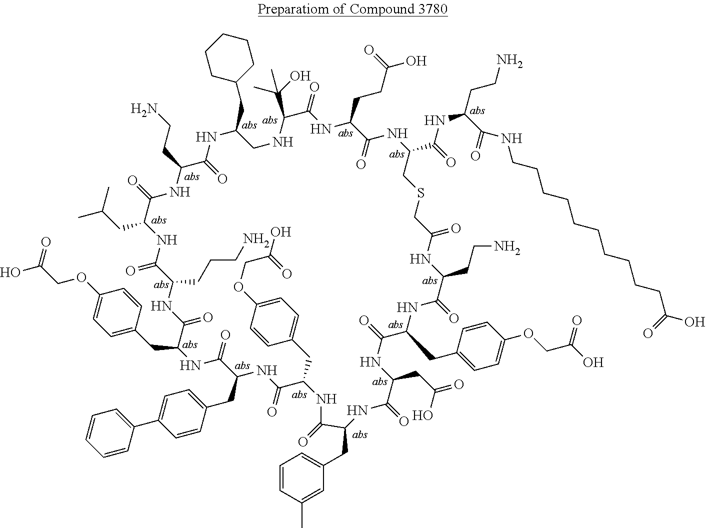 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3780 was prepared on a 50 μmol scale. The yield of the product was 14.6 mg, and its estimated purity by LCMS analysis was 96.2%. Analysis condition B: Retention time=1.56 min; ESI-MS(+) m/z [M+2H]²⁺: 1217.2.

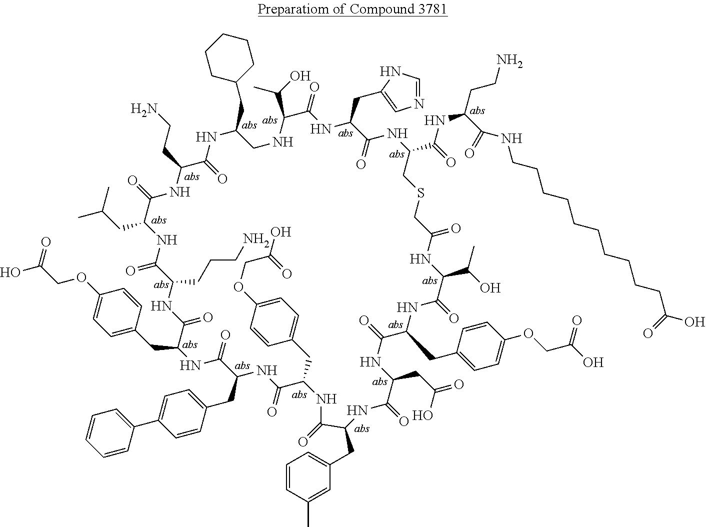 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3781 was prepared on a 50 μmol scale. The yield of the product was 56.1 mg, and its estimated purity by LCMS analysis was 88.1%. Analysis condition A: Retention time=1.5 min; ESI-MS(+) m/z [M+2H]²⁺: 1214.2.

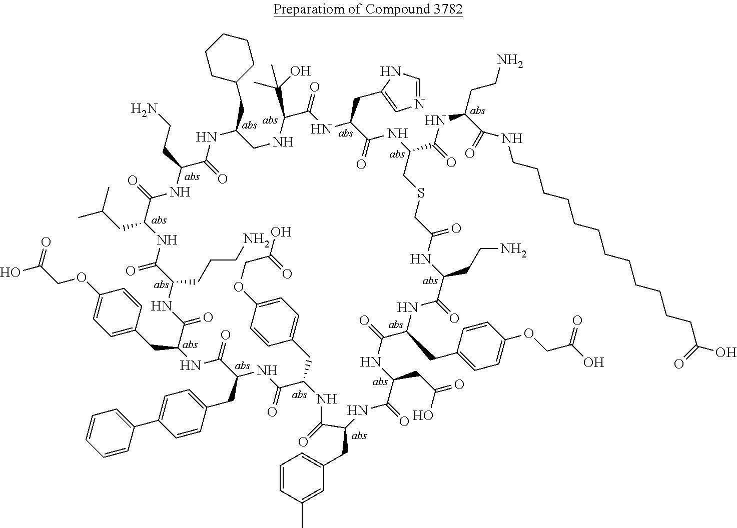 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCCCC(=O)O)NC(=O)[C@H](Cc3cnc[nH]3)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3782 was prepared on a 50 μmol scale. The yield of the product was 13.9 mg, and its estimated purity by LCMS analysis was 89.8%. Analysis condition A: Retention time=1.52 min; ESI-MS(+) m/z [M+2H]²⁺: 1235.1.

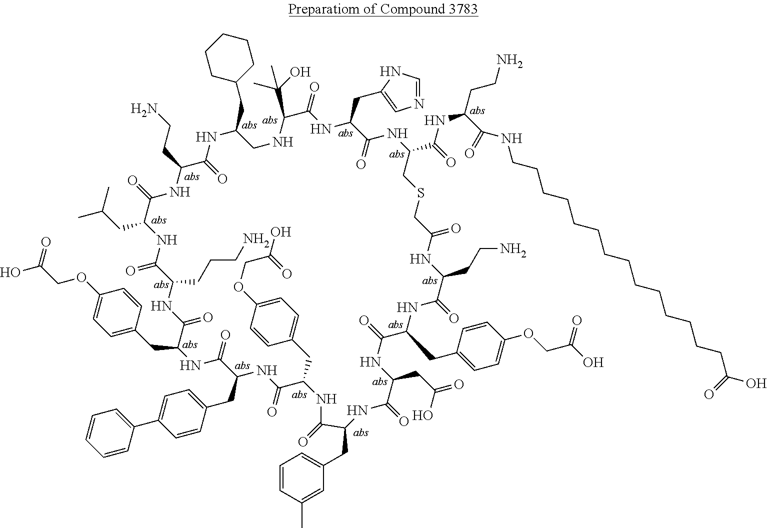 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCCCCCC(=O)O)NC(=O)[C@H](Cc3cnc[nH]3)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3783 was prepared on a 50 μmol scale. The yield of the product was 38 mg, and its estimated purity by LCMS analysis was 100%. Analysis condition A: Retention time=1.69 min; ESI-MS(+) m/z [M+2H]²⁺: 1249.1.

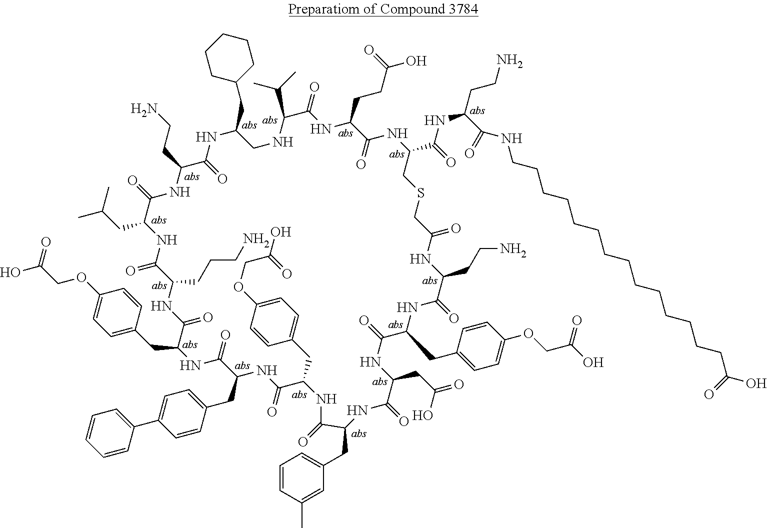 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCCCCCC(=O)O)NC(=O)[C@H](CCC(=O)O)NC(=O)[C@H](C(C)C)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3784 was prepared on a 50 μmol scale. The yield of the product was 15.4 mg, and its estimated purity by LCMS analysis was 100%. Analysis condition A: Retention time=1.73 min; ESI-MS(+) m/z [M+2H]²⁺: 1237.

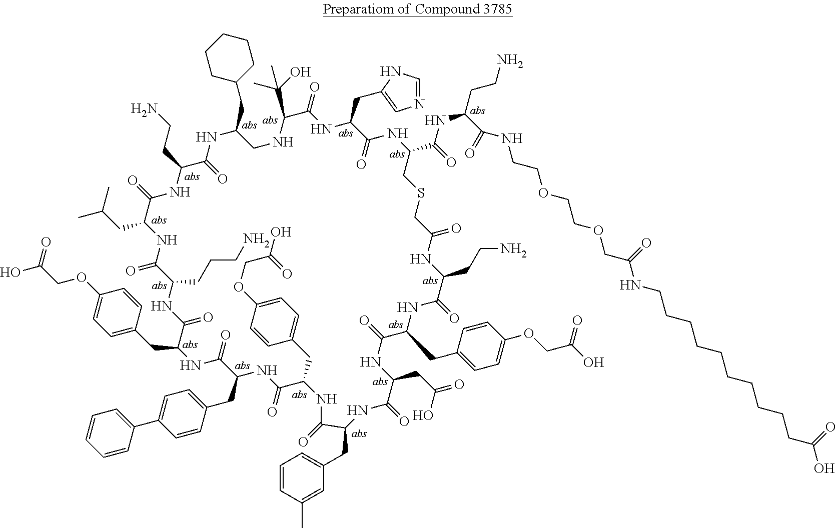 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](Cc3cnc[nH]3)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3785 was prepared on a 50 μmol scale. The yield of the product was 11.1 mg, and its estimated purity by LCMS analysis was 98.7%. Analysis condition B: Retention time=1.64 min; ESI-MS(+) m/z [M+3H]3²⁺: 862.9.

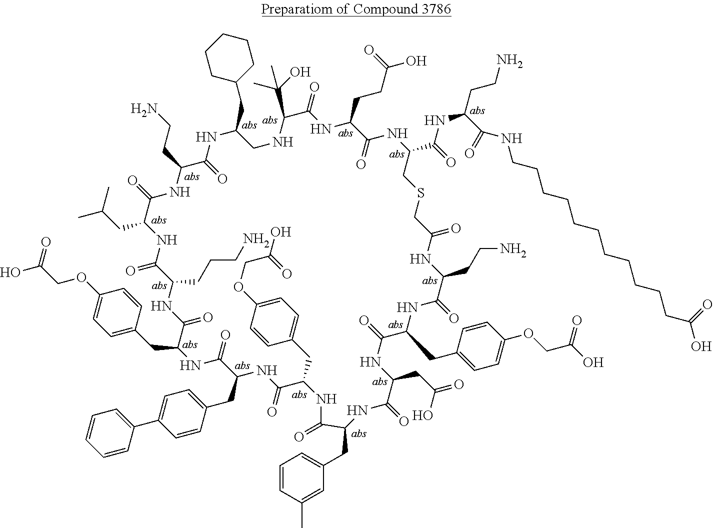 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCCC(=O)O)NC(=O)[C@H](CCC(=O)O)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3786 was prepared on a 50 μmol scale. The yield of the product was 25.4 mg, and its estimated purity by LCMS analysis was 95%. Analysis condition A: Retention time=1.51 min; ESI-MS(+) m/z [M+2H]²⁺: 1223.9.

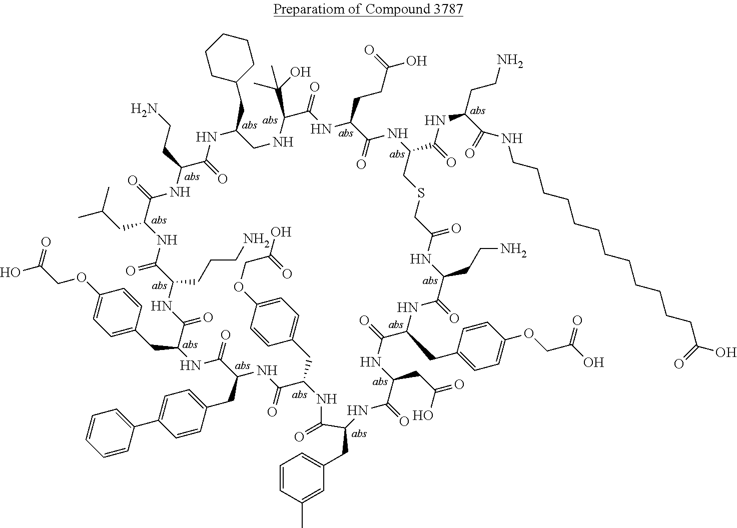 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCCCCCCCCCCCC(=O)O)NC(=O)[C@H](CCC(=O)O)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3787 was prepared on a 50 μmol scale. The yield of the product was 38.6 mg, and its estimated purity by LCMS analysis was 100%. Analysis condition A: Retention time=1.49 min; ESI-MS(+) m/z [M+2H]²⁺: 1231.1.

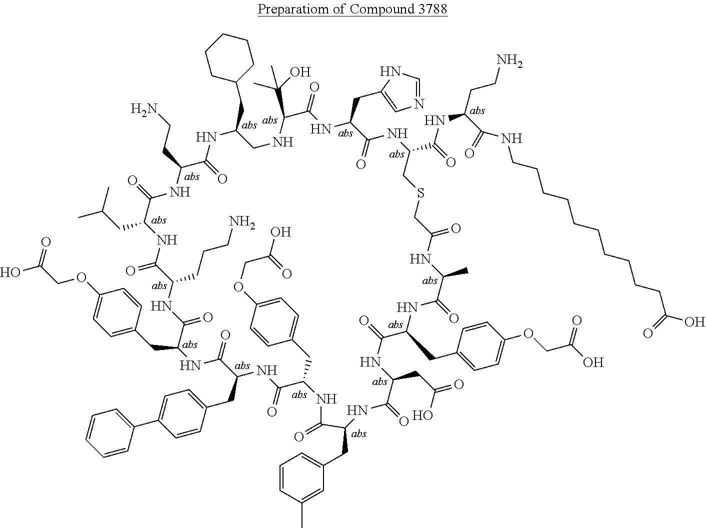 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3788 was prepared on a 50 μmol scale. The yield of the product was 9.7 mg, and its estimated purity by LCMS analysis was 95.6%. Analysis condition B: Retention time=1.69 min; ESI-MS(+) m/z [M+2H]²⁺: 1206.3.

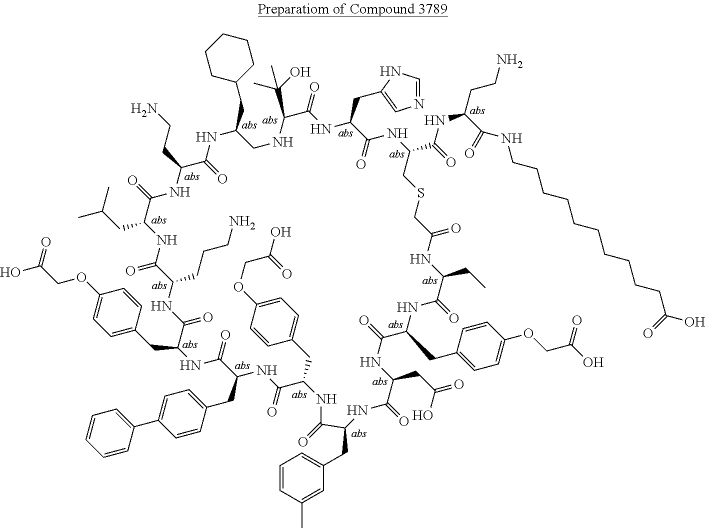 [SMILES: CC[C@@H]1NC(=O)CSC[C@@H](C(=O)N[C@@H](CCN)C(=O)NCCCCCCCCCCC(=O)O)NC(=O)[C@H](Cc2cnc[nH]2)NC(=O)[C@H](C(C)(C)O)NC[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3789 was prepared on a 50 μmol scale. The yield of the product was 15.4 mg, and its estimated purity by LCMS analysis was 95.4%. Analysis condition A: Retention time=1.52 min; ESI-MS(+) m/z [M+3H]³⁺: 809.1.

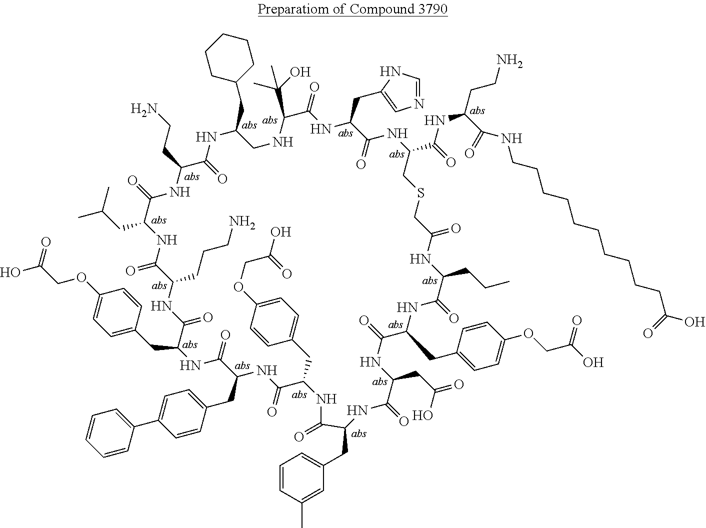 [SMILES: CCC[C@@H]1NC(=O)CSC[C@@H](C(=O)N[C@@H](CCN)C(=O)NCCCCCCCCCCC(=O)O)NC(=O)[C@H](Cc2cnc[nH]2)NC(=O)[C@H](C(C)(C)O)NC[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3790 was prepared on a 50 μmol scale. The yield of the product was 25.4 mg, and its estimated purity by LCMS analysis was 96.8%. Analysis condition A: Retention time=1.59 min; ESI-MS(+) m/z [M+3H]³⁺: 813.4.

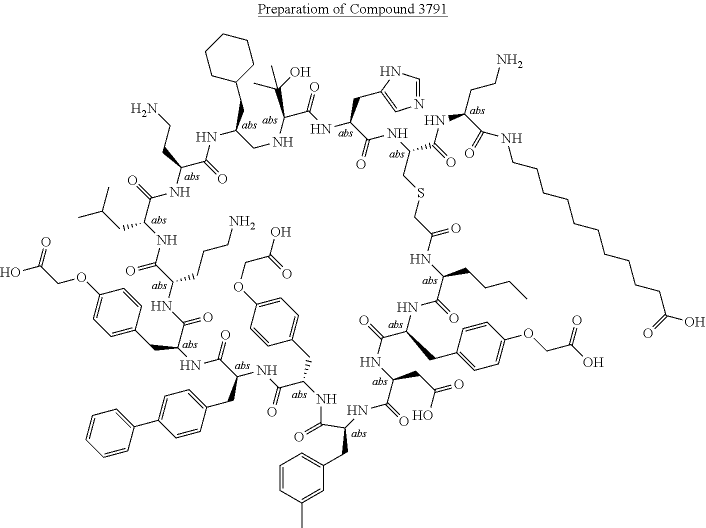 [SMILES: CCCC[C@@H]1NC(=O)CSC[C@@H](C(=O)N[C@@H](CCN)C(=O)NCCCCCCCCCCC(=O)O)NC(=O)[C@H](Cc2cnc[nH]2)NC(=O)[C@H](C(C)(C)O)NC[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3791 was prepared on a 50 μmol scale. The yield of the product was 11.2 mg, and its estimated purity by LCMS analysis was 96%. Analysis condition A: Retention time=1.61 min; ESI-MS(+) m/z [M+2H]²⁺: 1227.8.

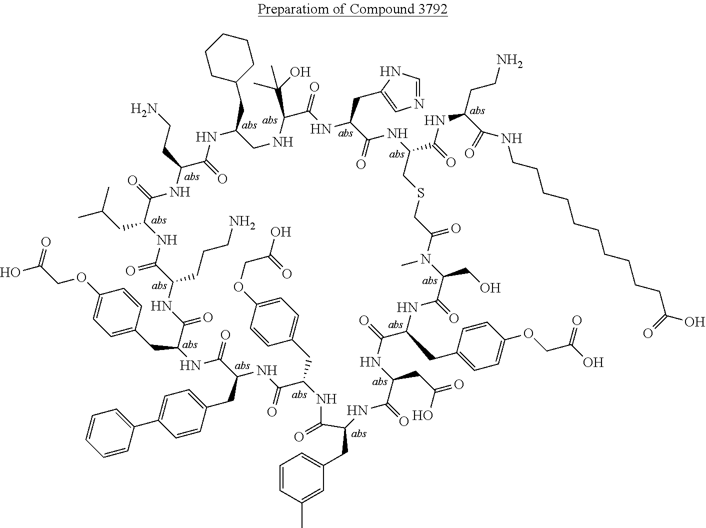 [SMILES: Cc1cccc(C[C@@H]2NC(=O)[C@H](CC(=O)O)NC(=O)[C@H](Cc3ccc(OCC(=O)O)cc3)NC(=O)[C@H](CO)N(C)C(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3792 was prepared on a 50 μmol scale. The yield of the product was 13.8 mg, and its estimated purity by LCMS analysis was 98.8%. Analysis condition A: Retention time=1.51 min; ESI-MS(+) m/z [M+2H]²⁺: 1222.

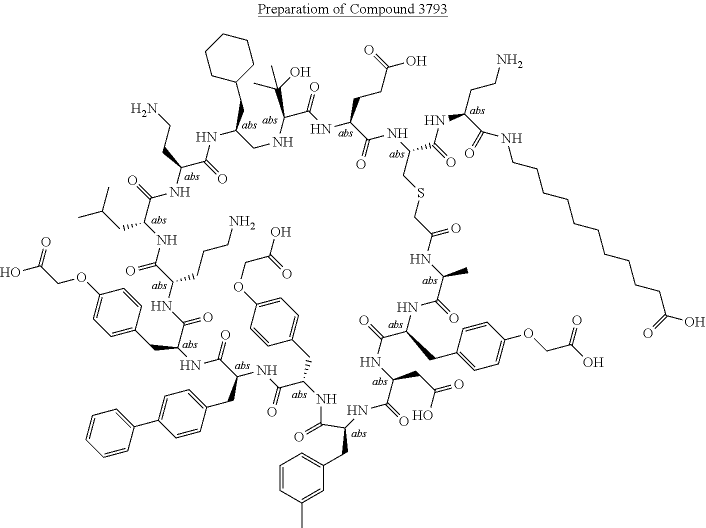 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCCCCCCCCCC(=O)O)NC(=O)[C@H](CCC(=O)O)NC(=O)[C@H](C(C)(C)O)NC[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3793 was prepared on a 50 μmol scale. The yield of the product was 37.9 mg, and its estimated purity by LCMS analysis was 98.8%. Analysis condition B: Retention time=1.73 min; ESI-MS(+) m/z [M+3H]³⁺: 802.2.

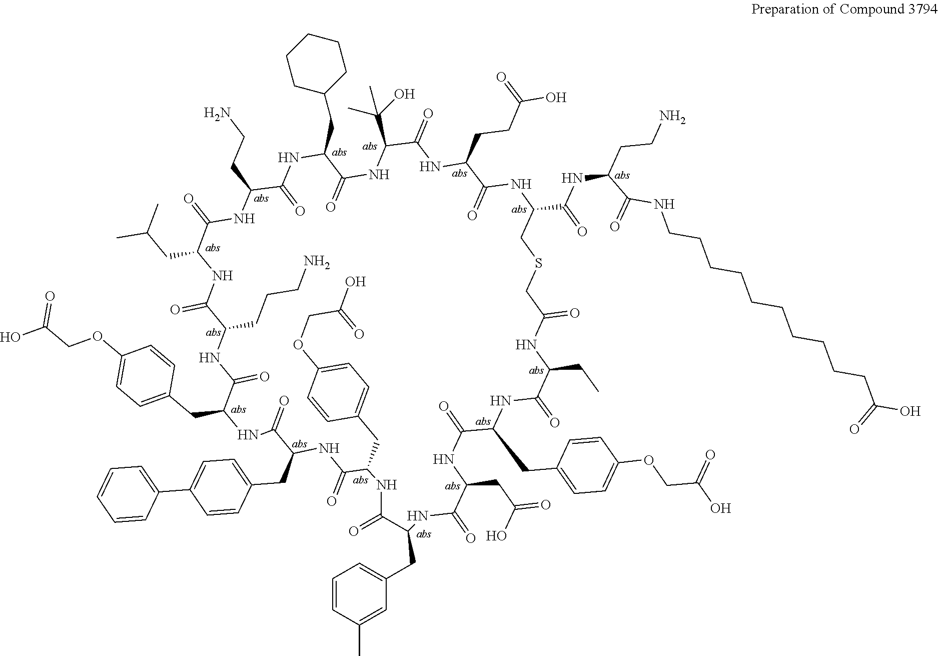 [SMILES: CC[C@@H]1NC(=O)CSC[C@@H](C(=O)N[C@@H](CCN)C(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3794 was prepared on a 50 μmol scale. The yield of the product was 7.2 mg, and its estimated purity by LCMS analysis was 100%. Analysis condition A: Retention time=1.42 min; ESI-MS(+) m/z [M+3H]³⁺: 807.1.

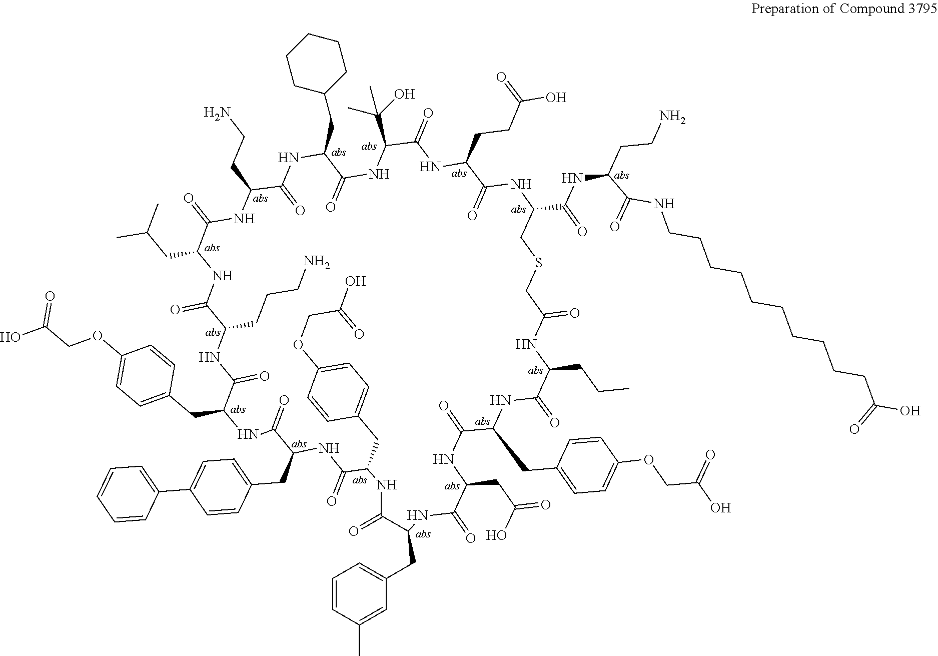 [SMILES: CCC[C@@H]1NC(=O)CSC[C@@H](C(=O)N[C@@H](CCN)C(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3795 was prepared on a 50 μmol scale. The yield of the product was 23.1 mg, and its estimated purity by LCMS analysis was 96.6%. Analysis condition B: Retention time=1.78 min; ESI-MS(+) m/z [M+2H]²⁺: 1216.3.

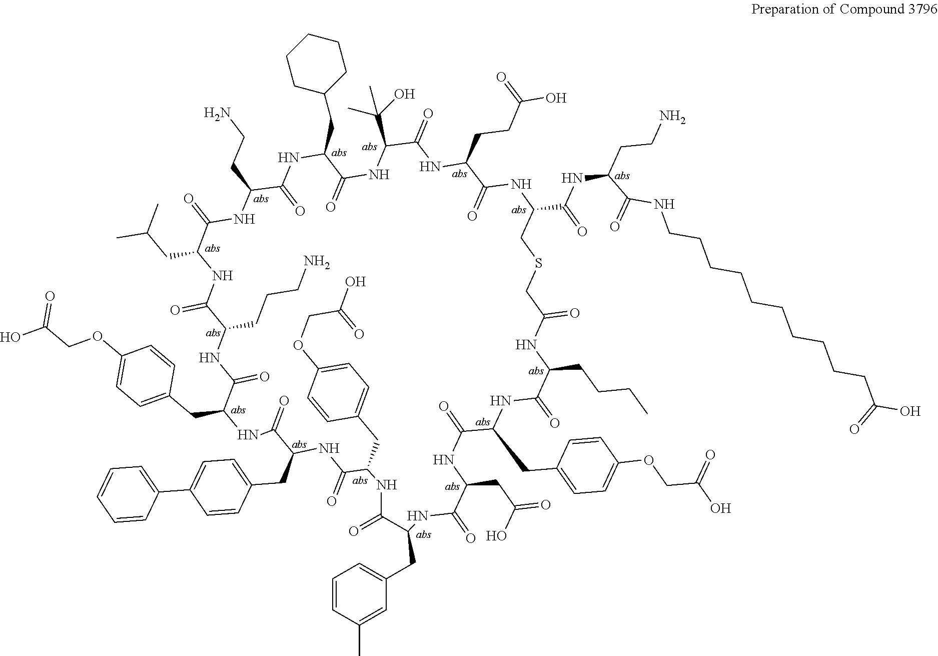 [SMILES: CCCC[C@@H]1NC(=O)CSC[C@@H](C(=O)N[C@@H](CCN)C(=O)NCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3796 was prepared on a 50 μmol scale. The yield of the product was 24.3 mg, and its estimated purity by LCMS analysis was 100%. Analysis condition A: Retention time=1.46 min; ESI-MS(+) m/z [M+2H]²⁺: 1223.2.

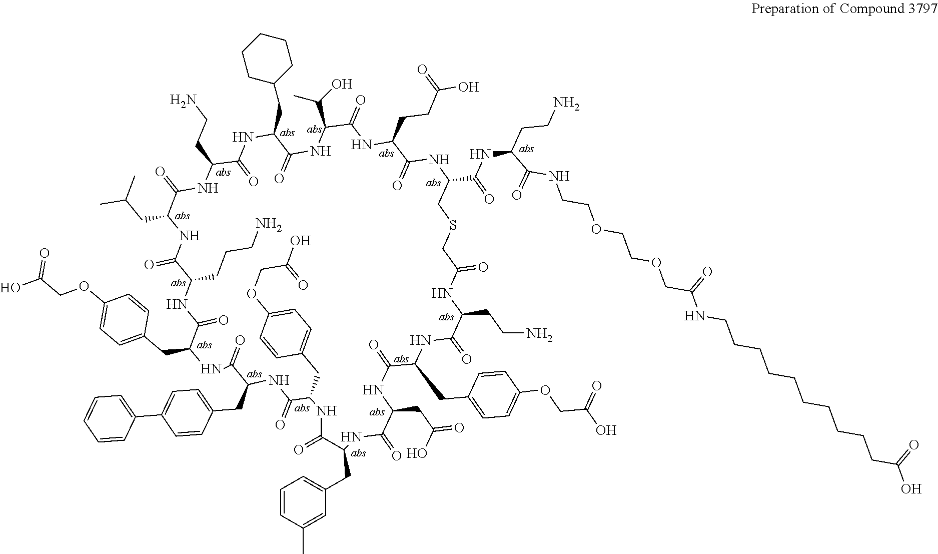 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3797 was prepared on a 50 μmol scale. The yield of the product was 4.8 mg, and its estimated purity by LCMS analysis was 96.2%. Analysis condition A: Retention time=1.52 min; ESI-MS(+) m/z [M+3H]³⁺: 855.2.

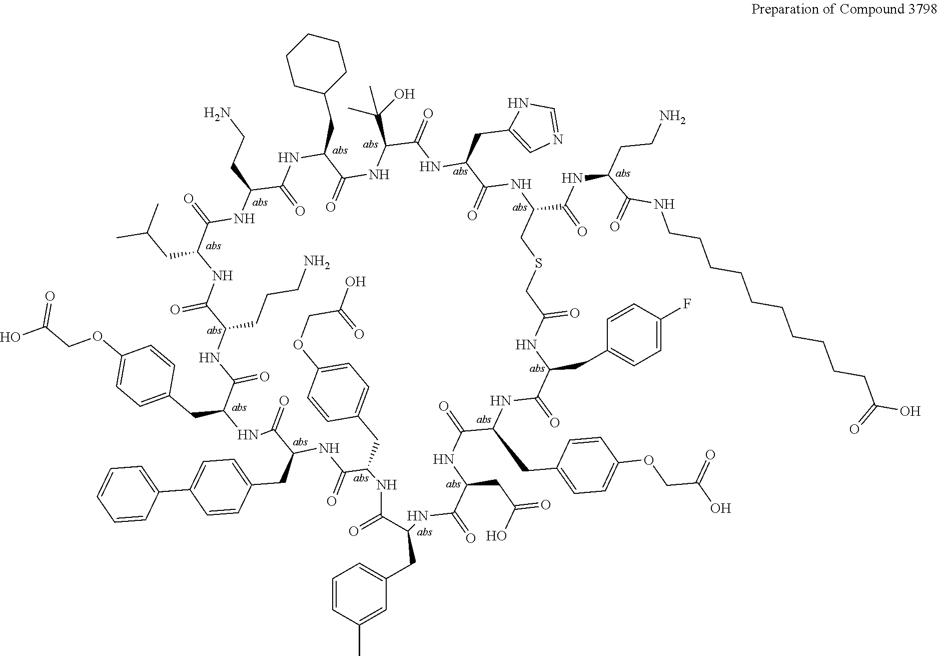 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3798 was prepared on a 50 μmol scale. The yield of the product was 43.3 mg, and its estimated purity by LCMS analysis was 93.8%. Analysis condition A: Retention time=1.67 min; ESI-MS(+) m/z [M+3H]³⁺: 836.2.

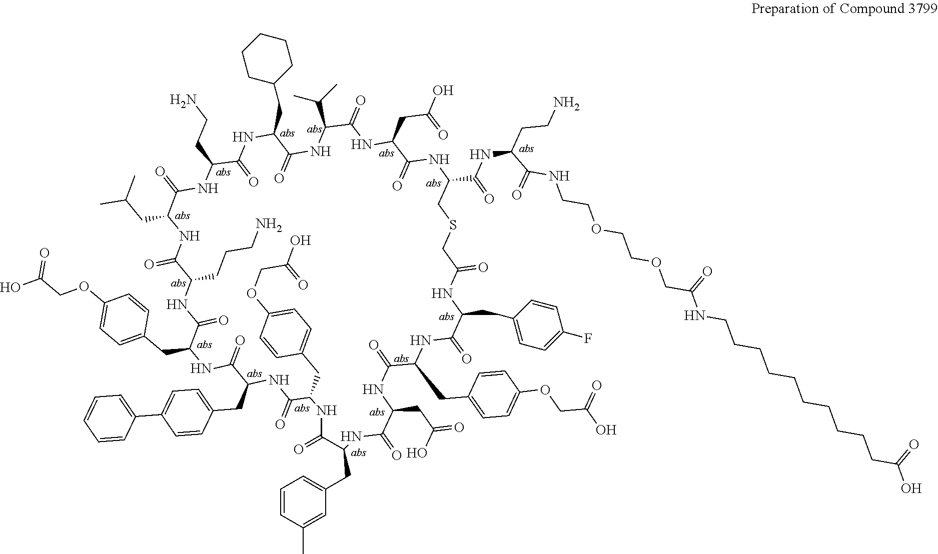 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCOCCOCC(=O)NCCCCCCCCCCC(=O)O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3799 was prepared on a 50 μmol scale. The yield of the product was 15.8 mg, and its estimated purity by LCMS analysis was 100%. Analysis condition A: Retention time=1.53 min; ESI-MS(+) m/z [M+2H]²⁺: 1307.1.

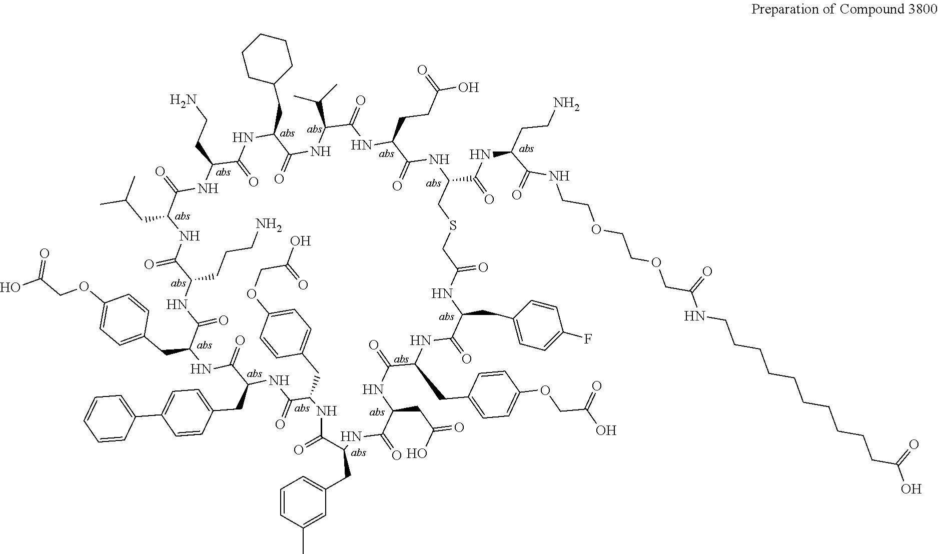 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3800 was prepared on a 50 μmol scale. The yield of the product was 8.5 mg, and its estimated purity by LCMS analysis was 96.5%. Analysis condition A: Retention time=1.55 min; ESI-MS(+) m/z [M+2H]²⁺: 1314.

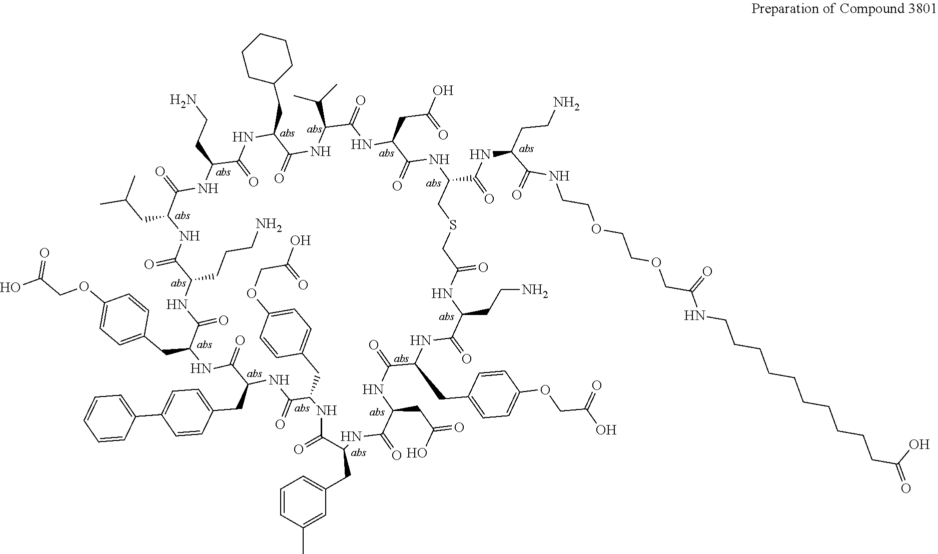 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3801 was prepared on a 50 μmol scale. The yield of the product was 20.6 mg, and its estimated purity by LCMS analysis was 96.2%. Analysis condition A: Retention time=1.51 min; ESI-MS(+) m/z [M+3H]³⁺: 850.3.

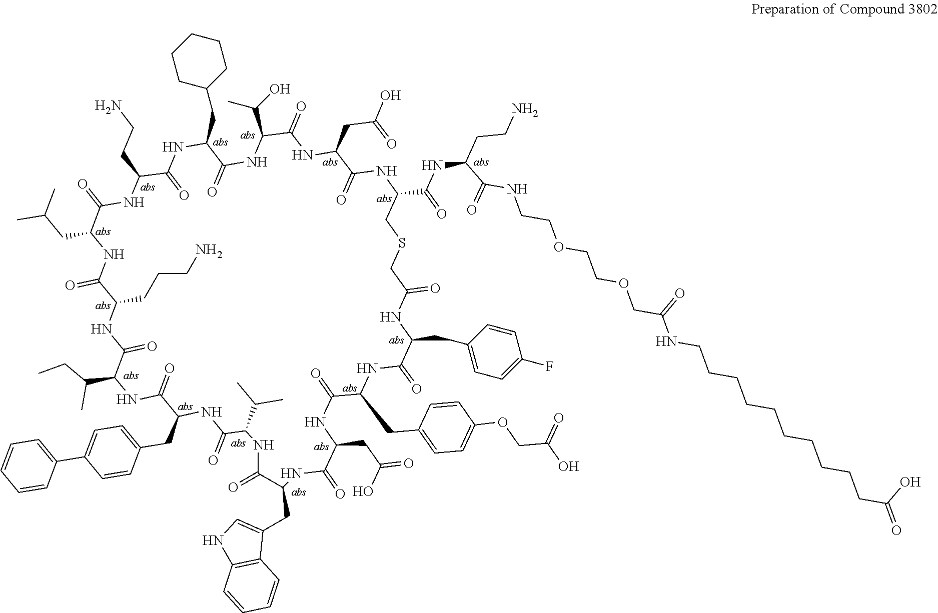 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(F)cc2)NC(=O)CSC[C@@H](C(=O)N[C@@H](CCN)C(=O)NCCOCCOCC(=O)NCCCCCCCCCCC(=O)O)NC(=O)[C@H](CC(=O)O)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC1=O]

Compound 3802 was prepared on a 50 μmol scale. The yield of the product was 29.3 mg, and its estimated purity by LCMS analysis was 100%. Analysis condition A: Retention time=1.77 min; ESI-MS(+) m/z [M+2H]²⁺: 1205.4.

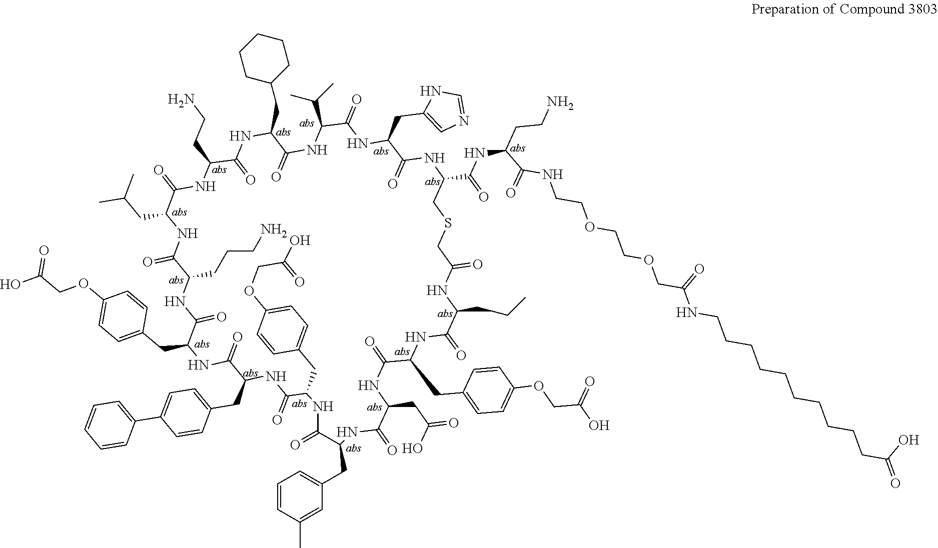 [SMILES: CCC[C@@H]1NC(=O)CSC[C@@H](C(=O)N[C@@H](CCN)C(=O)NCCOCCOCC(=O)NCCCCCCCCCCC(=O)O)NC(=O)[C@H](Cc2cnc[nH]2)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3803 was prepared on a 50 μmol scale. The yield of the product was 19 mg, and its estimated purity by LCMS analysis was 95.5%. Analysis condition B: Retention time=1.71 min; ESI-MS(+) m/z [M+2H]²⁺: 1285.1.

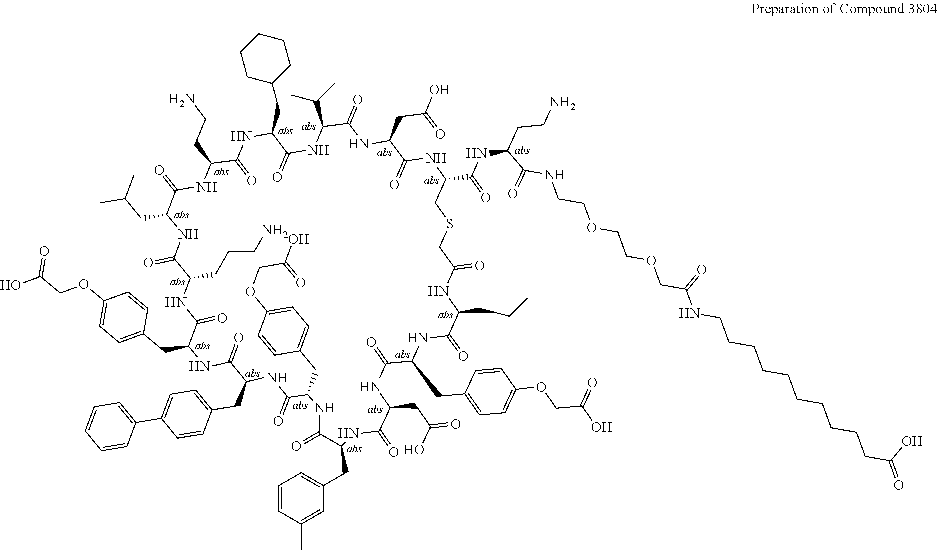 [SMILES: CCC[C@@H]1NC(=O)CSC[C@@H](C(=O)N[C@@H](CCN)C(=O)NCCOCCOCC(=O)NCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3804 was prepared on a 50 μmol scale. The yield of the product was 13.4 mg, and its estimated purity by LCMS analysis was 95.8%. Analysis condition B: Retention time=1.81 min; ESI-MS(+) m/z [M+3H]³⁺: 850.

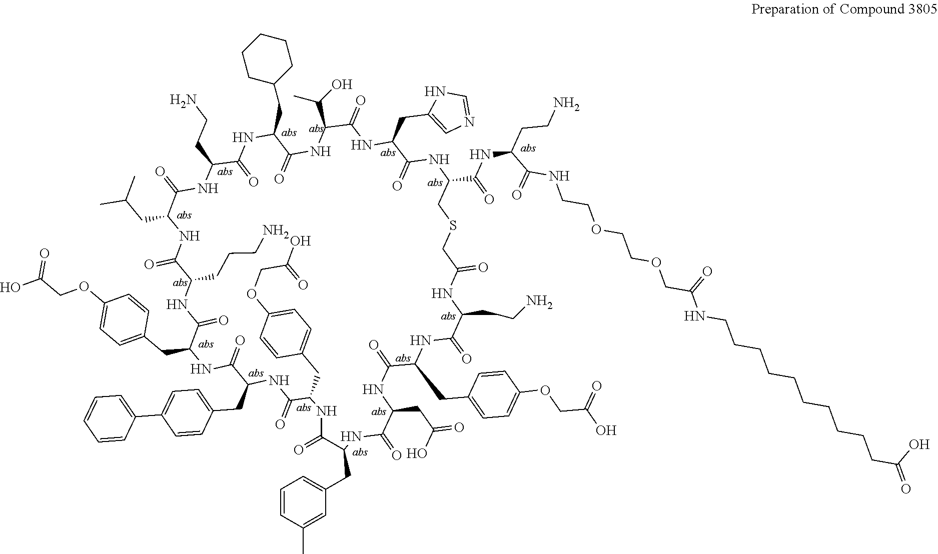 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCCCCCCCCCC(=O)O)NC(=O)[C@H](Cc3cnc[nH]3)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3805 was prepared on a 50 μmol scale. The yield of the product was 15.4 mg, and its estimated purity by LCMS analysis was 95.5%. Analysis condition B: Retention time=1.74 min; ESI-MS(+) m/z [M+3H]³⁺: 858.2.

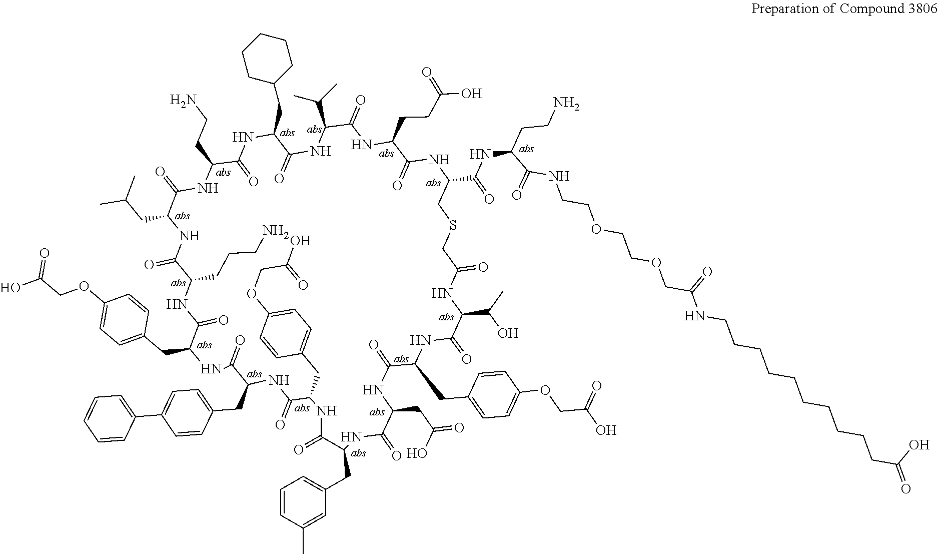 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3806 was prepared on a 50 μmol scale. The yield of the product was 3.3 mg, and its estimated purity by LCMS analysis was 97%. Analysis condition B: Retention time=1.74 min; ESI-MS(+) m/z [M+2H]²⁺: 1282.1.

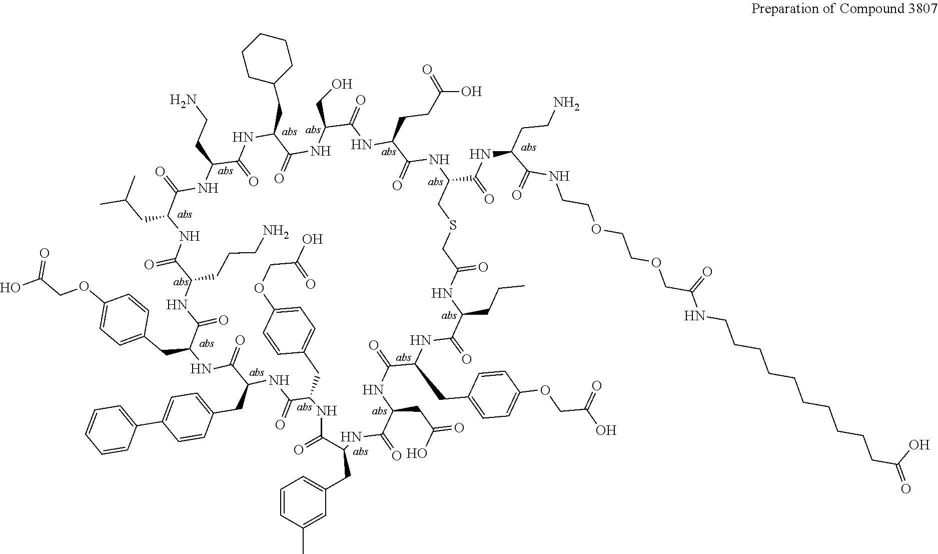 [SMILES: CCC[C@@H]1NC(=O)CSC[C@@H](C(=O)N[C@@H](CCN)C(=O)NCCOCCOCC(=O)NCCCCCCCCCCC(=O)O)NC(=O)[C@H](CCC(=O)O)NC(=O)[C@H](CO)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3807 was prepared on a 50 μmol scale. The yield of the product was 19.9 mg, and its estimated purity by LCMS analysis was 87.5%. Analysis condition A: Retention time=1.48 min; ESI-MS(+) m/z [M+3H]³⁺: 850.2.

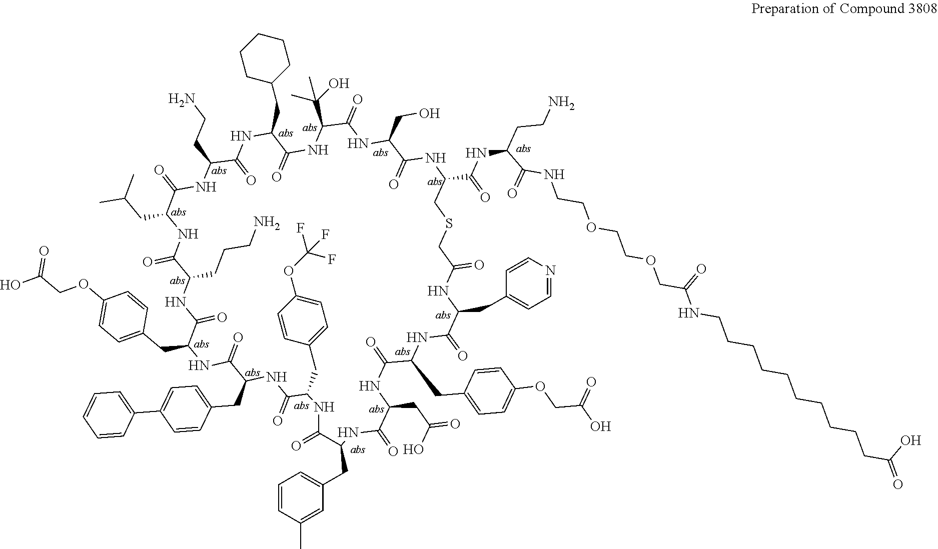 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(=O)NCCOCCOCC(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OC(F)(F)F)cc3)NC2=O)c1]

Compound 3808 was prepared on a 50 μmol scale. The yield of the product was 7.3 mg, and its estimated purity by LCMS analysis was 100%. Analysis condition A: Retention time=1.79 min; ESI-MS(+) m/z [M+2H]²⁺: 1225.2.

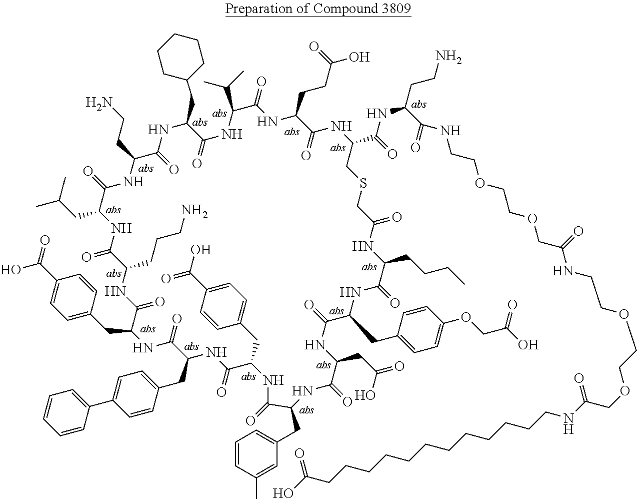 [SMILES: CCCC[C@@H]1NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3809 was prepared on a 50 μmol scale. The yield of the product was 31.2 mg, and its estimated purity by LCMS analysis was 97.1%. Analysis condition A: Retention time=1.62 min; ESI-MS(+) m/z [M+2H]²⁺: 1345.

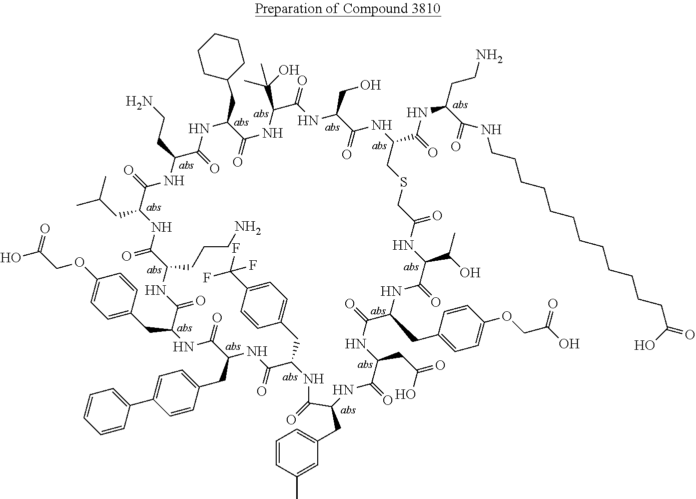 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(F)(F)F)cc3)NC2=O)c1]

Compound 3810 was prepared on a 50 μmol scale. The yield of the product was 19.7 mg, and its estimated purity by LCMS analysis was 95.3%. Analysis condition A: Retention time=1.85 min; ESI-MS(+) m/z [M+2H]²⁺: 1208.

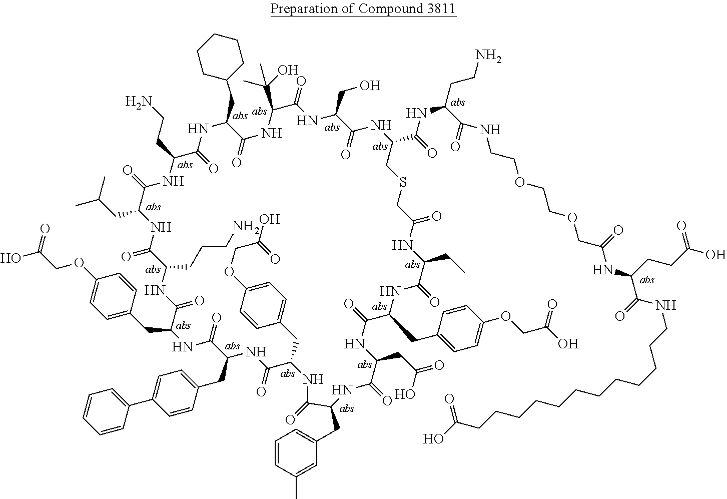 [SMILES: CC[C@@H]1NC(=O)CSC[C@@H](C(=O)N[C@@H](CCN)C(=O)NCCOCCOCC(=O)N[C@@H](CCC(=O)O)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3811 was prepared on a 50 μmol scale. The yield of the product was 23.2 mg, and its estimated purity by LCMS analysis was 91%. Analysis condition A: Retention time=1.47 min; ESI-MS(+) m/z [M+2H]²⁺: 1340.

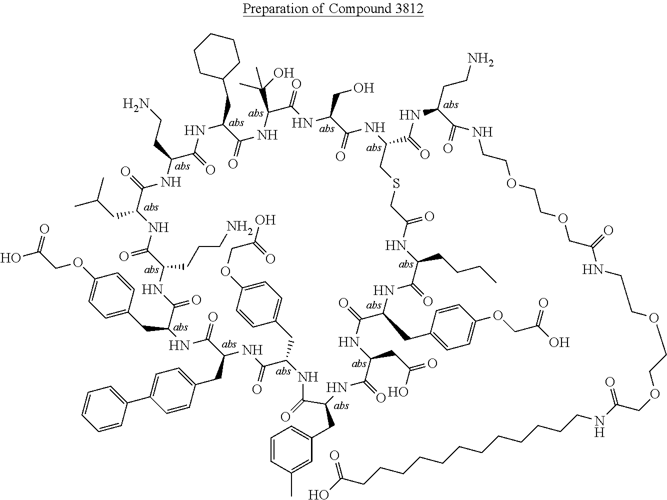 [SMILES: CCCC[C@@H]1NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3812 was prepared on a 50 μmol scale. The yield of the product was 63.8 mg, and its estimated purity by LCMS analysis was 95.6%. Analysis condition A: Retention time=1.6 min; ESI-MS(+) m/z [M+2H]²⁺: 1362.

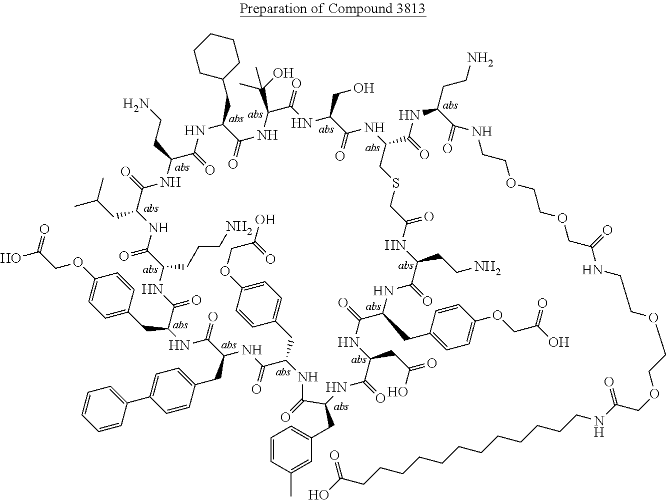 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3813 was prepared on a 50 μmol scale. The yield of the product was 22.2 mg, and its estimated purity by LCMS analysis was 96.3%. Analysis condition A: Retention time=1.67 min; ESI-MS(+) m/z [M+2H]²⁺: 1355.1.

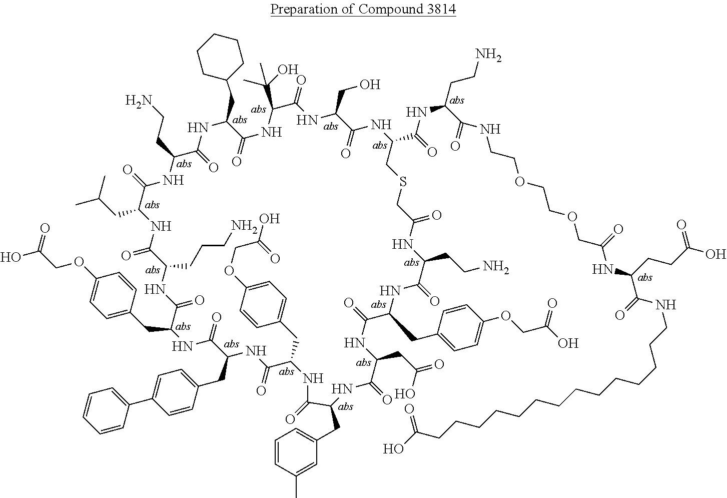 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)N[C@@H](CCC(=O)O)C(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3814 was prepared on a 50 μmol scale. The yield of the product was 12.8 mg, and its estimated purity by LCMS analysis was 95.4%. Analysis condition A: Retention time=1.63 min; ESI-MS(+) m/z [M+2H]²⁺: 1361.

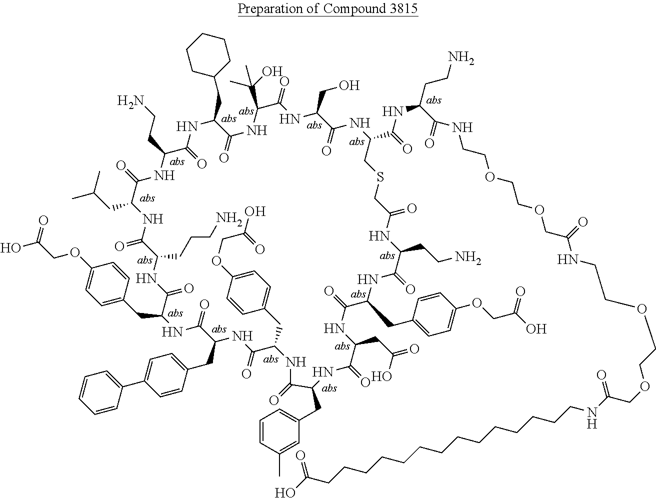 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3815 was prepared on a 50 μmol scale. The yield of the product was 15.8 mg, and its estimated purity by LCMS analysis was 98.6%. Analysis condition A: Retention time-1.72 min; ESI-MS(+) m/z [M+2H]²⁺: 1369.2.

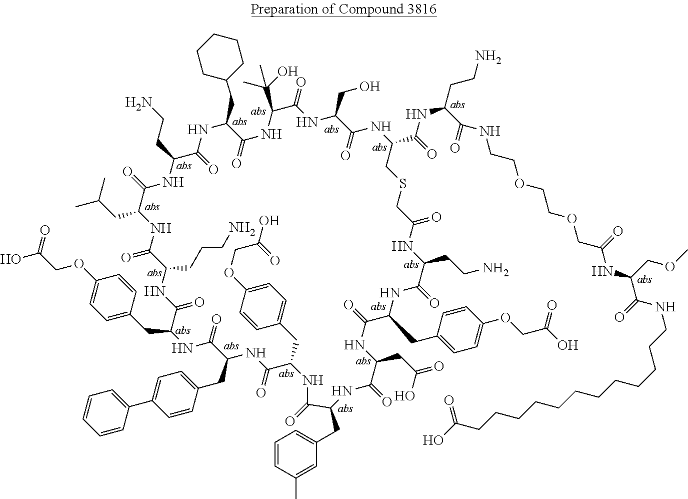 [SMILES: COC[C@H](NC(=O)COCCOCCNC(=O)[C@H](CCN)NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)NCCCCCCCCCCCCC(=O)O]

Compound 3816 was prepared on a 50 μmol scale. The yield of the product was 25.6 mg, and its estimated purity by LCMS analysis was 92.4%. Analysis condition A: Retention time=1.64 min; ESI-MS(+) m/z [M+2H]²⁺: 1333.

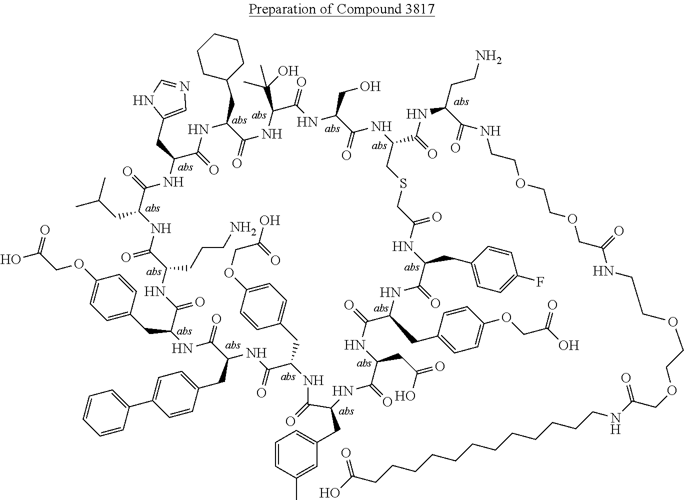 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3817 was prepared on a 50 μmol scale. The yield of the product was 27.7 mg, and its estimated purity by LCMS analysis was 89.6%. Analysis condition A: Retention time-1.62 min; ESI-MS(+) m/z [M+2H]²⁺: 1406.2.

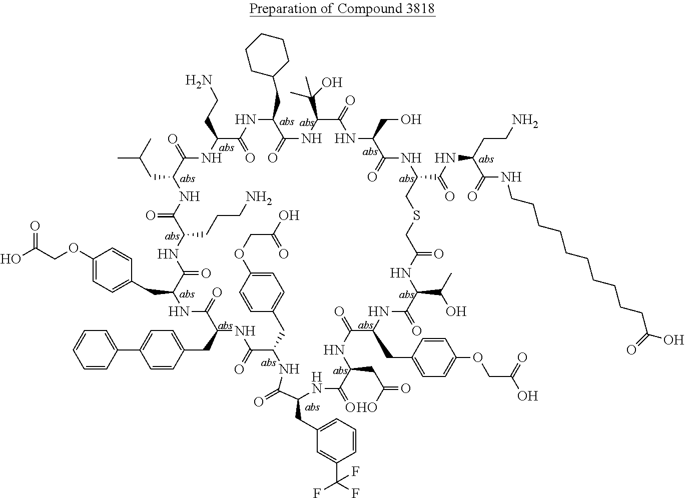 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3818 was prepared on a 50 μmol scale. The yield of the product was 13.5 mg, and its estimated purity by LCMS analysis was 100%. Analysis condition A: Retention time=1.6 min; ESI-MS(+) m/z [M+3H]³⁺: 815.9.

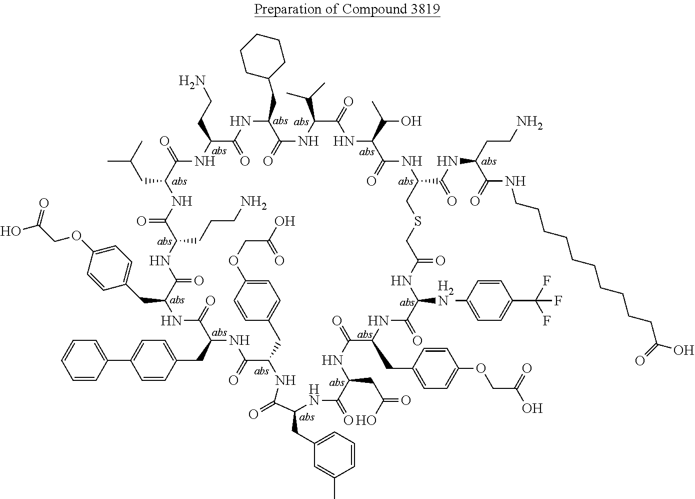

Compound 3819 was prepared on a 50 μmol scale. The yield of the product was 14.5 mg, and its estimated purity by LCMS analysis was 95%. Analysis condition B: Retention time=1.92 min; ESI-MS(+) m/z [M+3H]³⁺: 835.5.

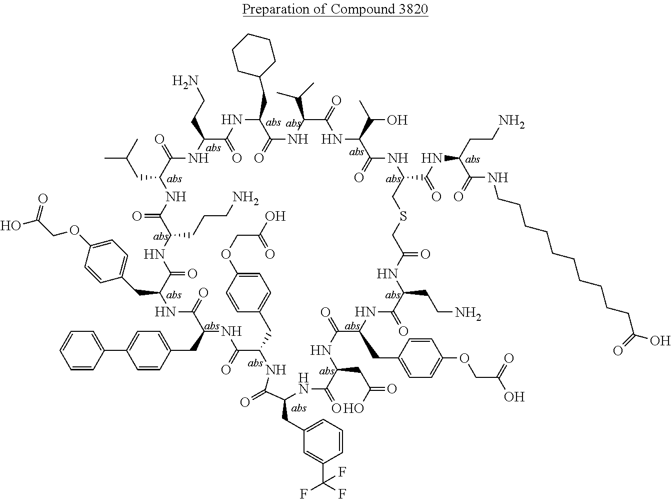 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3820 was prepared on a 50 μmol scale. The yield of the product was 15.1 mg, and its estimated purity by LCMS analysis was 88.9%. Analysis condition A: Retention time=1.75 min; ESI-MS(+) m/z [M+2H]²⁺: 1221.9.

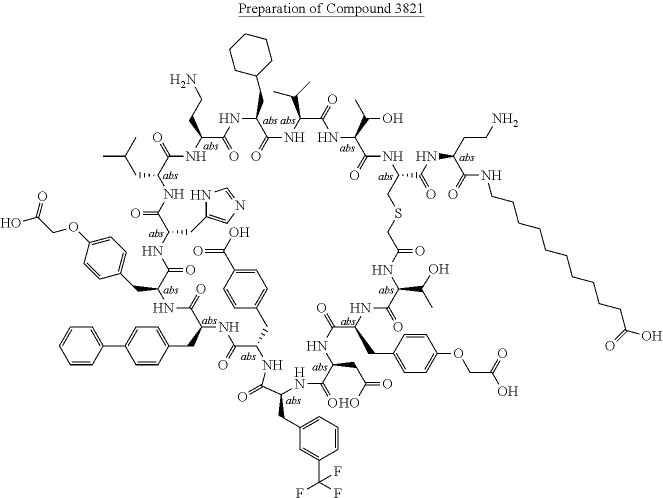 [SMILES: CC(C)C[C@H]1NC(=O)[C@H](Cc2cnc[nH]2)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCC(=O)O)NC(=O)[C@H](C(C)O)NC(=O)[C@H](C(C)C)NC(=O)[C@H](CC2CCCCC2)NC(=O)[C@H](CCN)NC1=O]

Compound 3821 was prepared on a 50 μmol scale. The yield of the product was 27.4 mg, and its estimated purity by LCMS analysis was 97.4%. Analysis condition A: Retention time-1.67 min; ESI-MS(+) m/z [M+2H]²⁺: 1218.9.

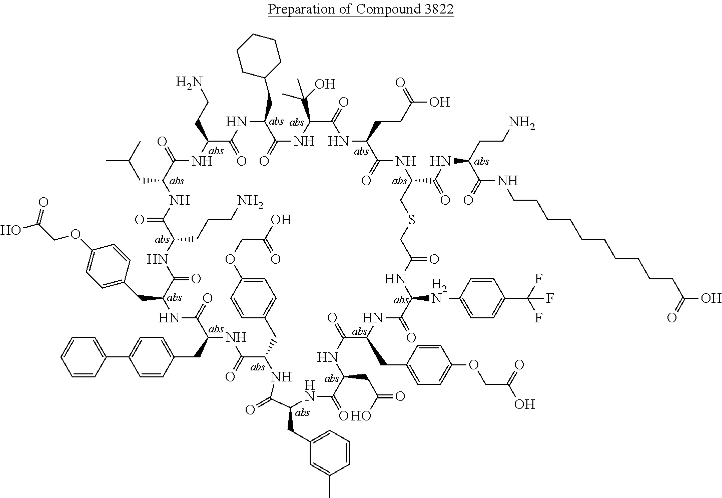

Compound 3822 was prepared on a 50 μmol scale. The yield of the product was 54.6 mg, and its estimated purity by LCMS analysis was 95.9%. Analysis condition B: Retention time=1.74 min; ESI-MS(+) m/z [M+2H]²⁺: 1274.3.

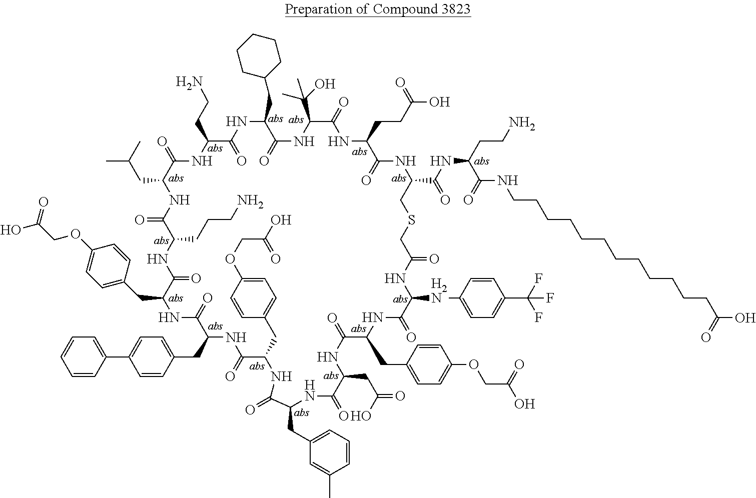

Compound 3823 was prepared on a 50 μmol scale. The yield of the product was 24.1 mg, and its estimated purity by LCMS analysis was 97.2%. Analysis condition A: Retention time=1.8 min; ESI-MS(+) m/z [M+3H]³⁺: 859.2.

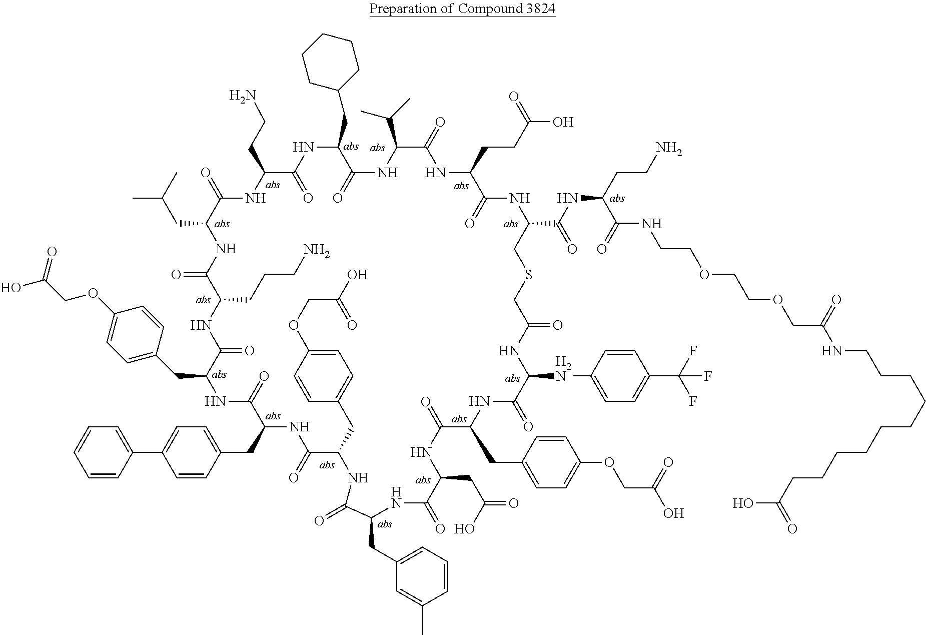

Compound 3824 was prepared on a 50 μmol scale. The yield of the product was 13.3 mg, and its estimated purity by LCMS analysis was 96.2%. Analysis condition A: Retention time=1.71 min; ESI-MS(+) m/z [M+2H]²⁺: 1339.3.

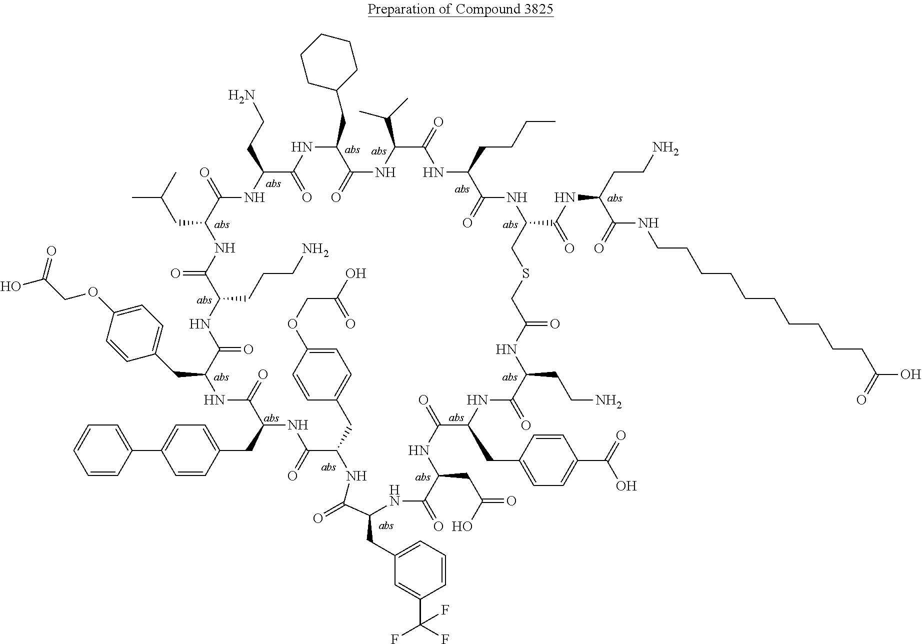 [SMILES: CCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CCN)NC(=O)CSC[C@@H](C(=O)N[C@@H](CCN)C(=O)NCCCCCCCCCCC(=O)O)NC1=O]

Compound 3825 was prepared on a 50 μmol scale. The yield of the product was 12.1 mg, and its estimated purity by LCMS analysis was 99%. Analysis condition A: Retention time=1.73 min; ESI-MS(+) m/z [M+2H]²⁺: 1213.2.

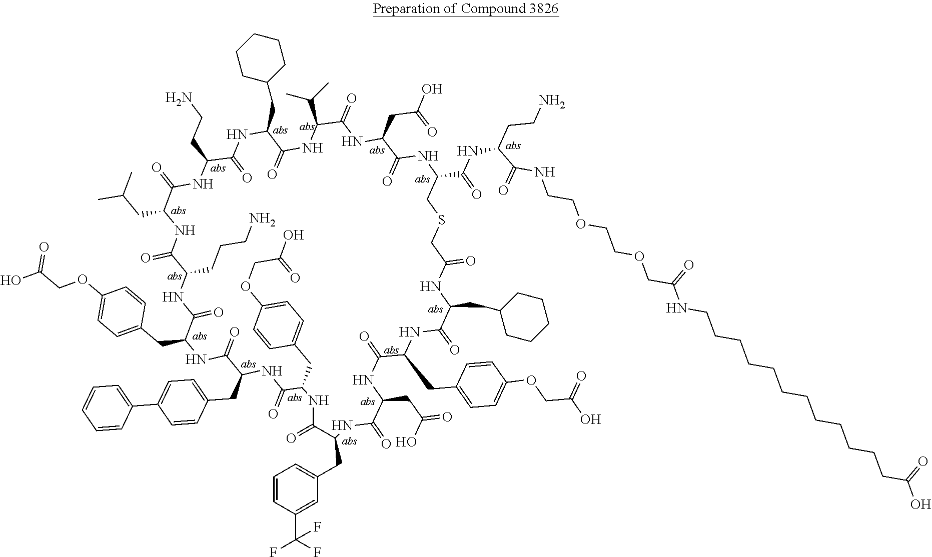 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CC2CCCCC2)NC(=O)CSC[C@@H](C(=O)N[C@H](CCN)C(=O)NCCOCCOCC(=O)NCCCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC1=O]

Compound 3826 was prepared on a 50 μmol scale. The yield of the product was 13.1 mg, and its estimated purity by LCMS analysis was 92%. Analysis condition B: Retention time=2.07 min; ESI-MS(+) m/z [M+2H]²⁺: 1342.3.

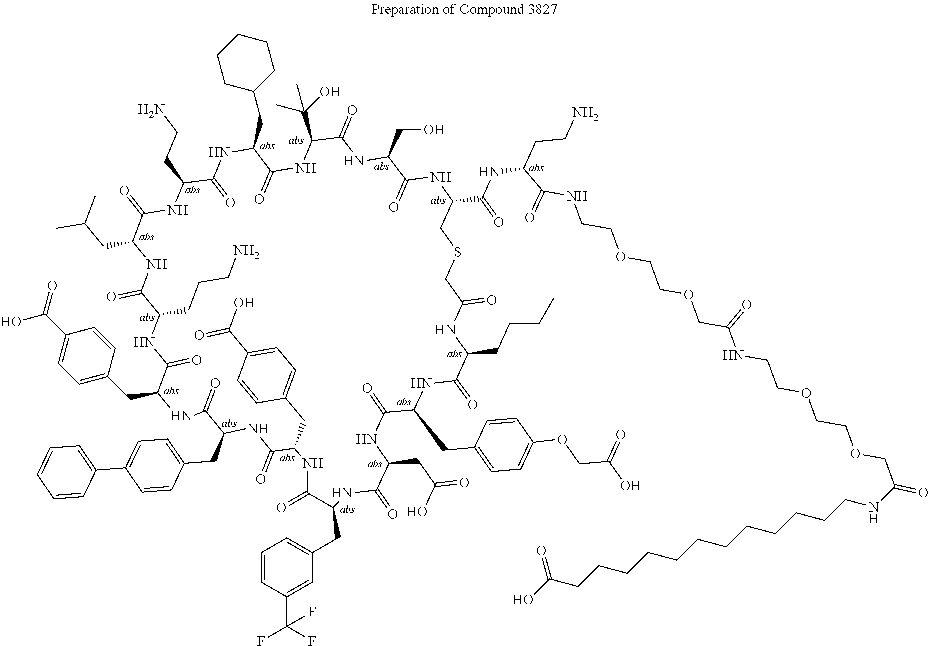 [SMILES: CCCC[C@@H]1NC(=O)CSC[C@@H](C(=O)N[C@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3827 was prepared on a 50 μmol scale. The yield of the product was 16.6 mg, and its estimated purity by LCMS analysis was 97%. Analysis condition B: Retention time=1.84 min; ESI-MS(+) m/z [M+2H]²⁺: 1358.3.

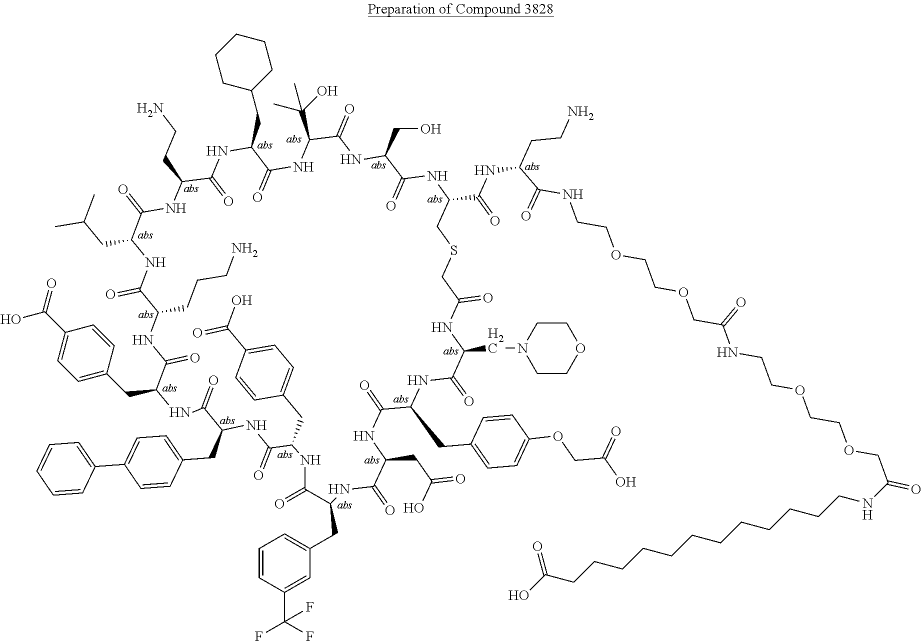 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3828 was prepared on a 50 μmol scale. The yield of the product was 19.7 mg, and its estimated purity by LCMS analysis was 96%. Analysis condition B: Retention time=1.87 min; ESI-MS(+) m/z [M+2H]²⁺: 1380.

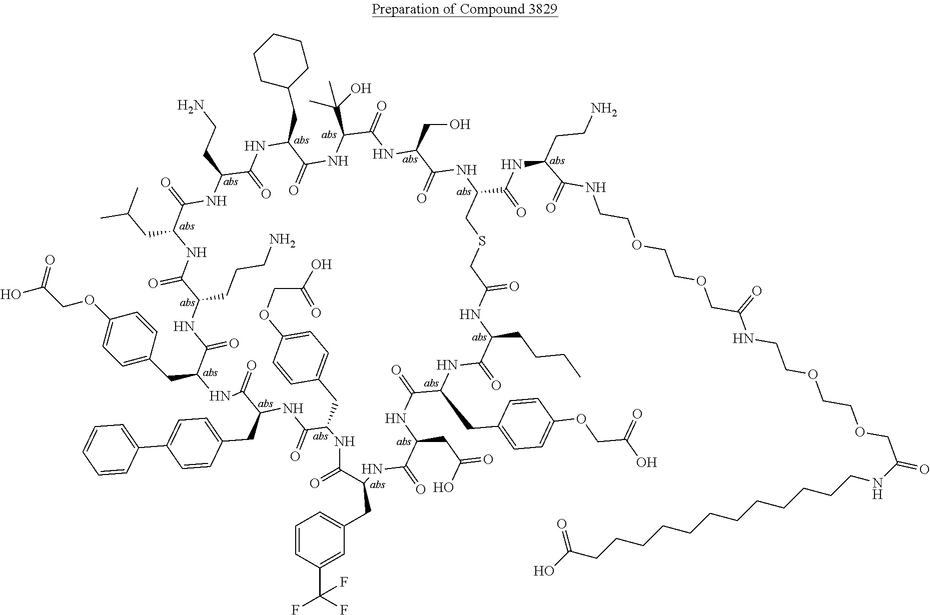 [SMILES: CCCC[C@@H]1NC(=O)CSC[C@@H](C(=O)N[C@@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3829 was prepared on a 50 μmol scale. The yield of the product was 35.8 mg, and its estimated purity by LCMS analysis was 88.2%. Analysis condition A: Retention time=1.68 min; ESI-MS(+) m/z [M+3H]³⁺: 926.

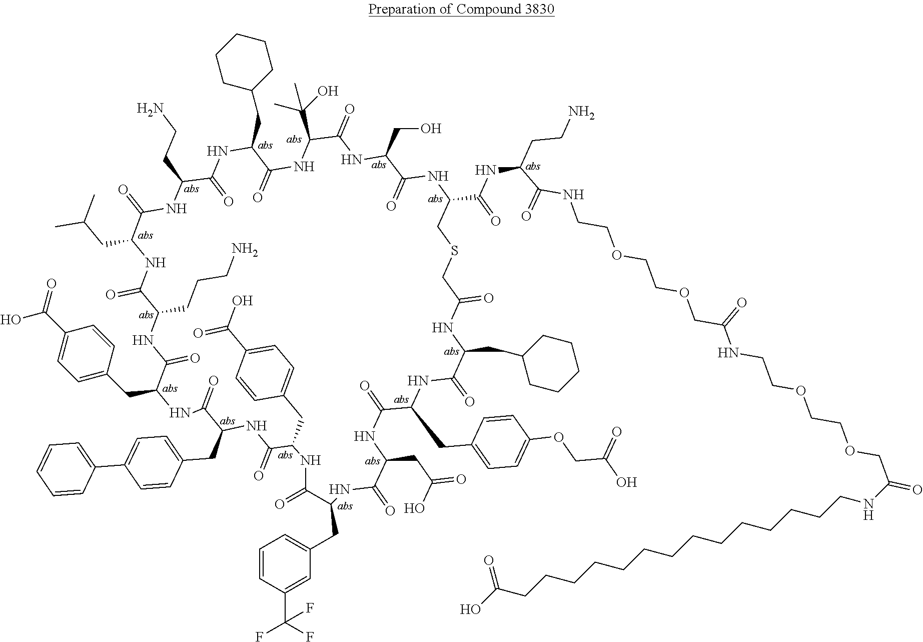 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CC2CCCCC2)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3830 was prepared on a 50 μmol scale. The yield of the product was 6.8 mg, and its estimated purity by LCMS analysis was 98.3%. Analysis condition A: Retention time=2.1 min; ESI-MS(+) m/z [M+3H]³⁺: 928.9.

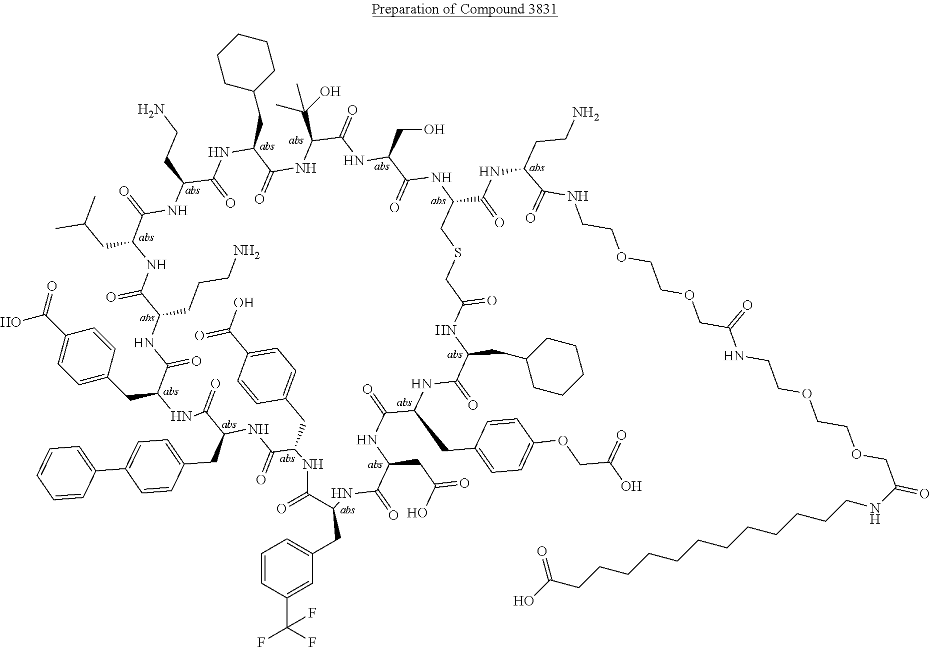 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CC2CCCCC2)NC(=O)CSC[C@@H](C(=O)N[C@H](CCN)C(=O)NCCOCCOCC(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3831 was prepared on a 50 μmol scale. The yield of the product was 20.7 mg, and its estimated purity by LCMS analysis was 87.3%. Analysis condition A: Retention time=1.7 min; ESI-MS(+) m/z [M+2H]²⁺: 1379.

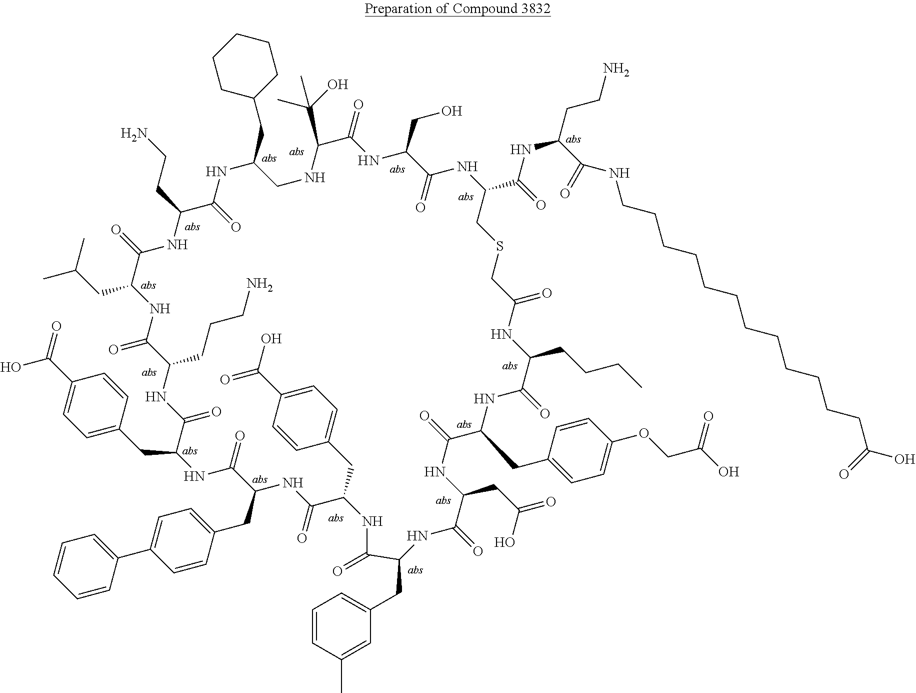 [SMILES: CCCC[C@@H]1NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3832 was prepared on a 50 μmol scale. The yield of the product was 17 mg, and its estimated purity by LCMS analysis was 90.3%. Analysis condition A: Retention time=1.72 min; ESI-MS(+) m/z [M+2H]²⁺: 1213.9.

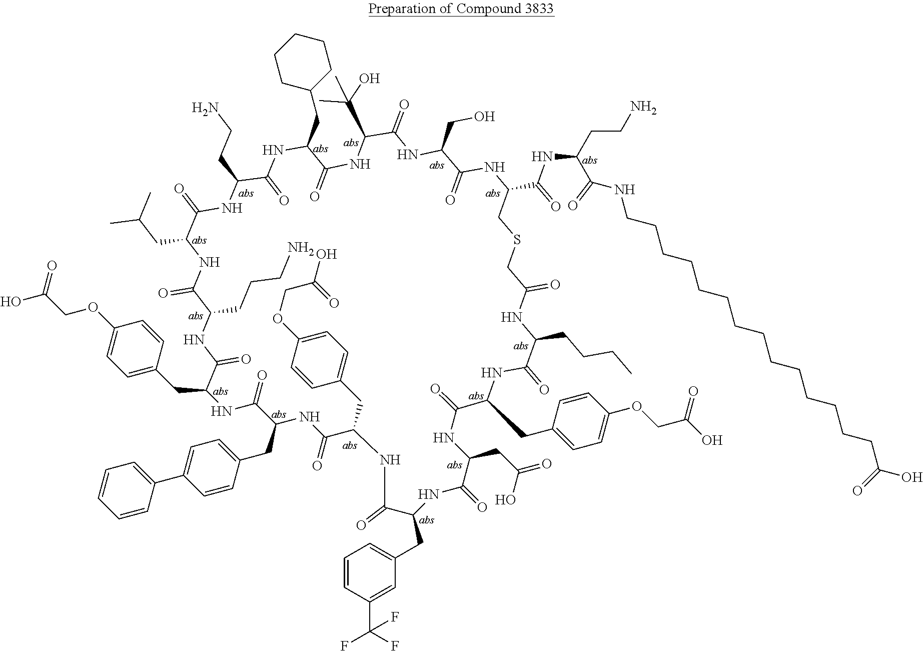 [SMILES: CCCC[C@@H]1NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3833 was prepared on a 50 μmol scale. The yield of the product was 48.2 mg, and its estimated purity by LCMS analysis was 95%. Analysis condition A: Retention time=1.72 min; ESI-MS(+) m/z [M+3H]³⁺: 829.1.

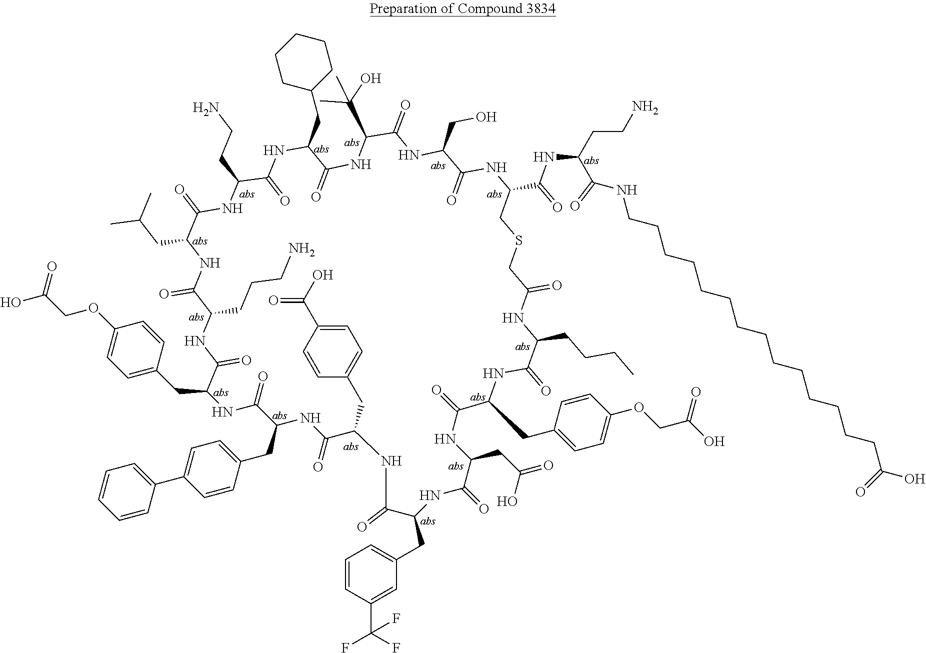 [SMILES: CCCC[C@@H]1NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3834 was prepared on a 50 μmol scale. The yield of the product was 46.4 mg, and its estimated purity by LCMS analysis was 87.3%. Analysis condition A: Retention time=1.73 min; ESI-MS(+) m/z [M+2H]²⁺: 1228.3.

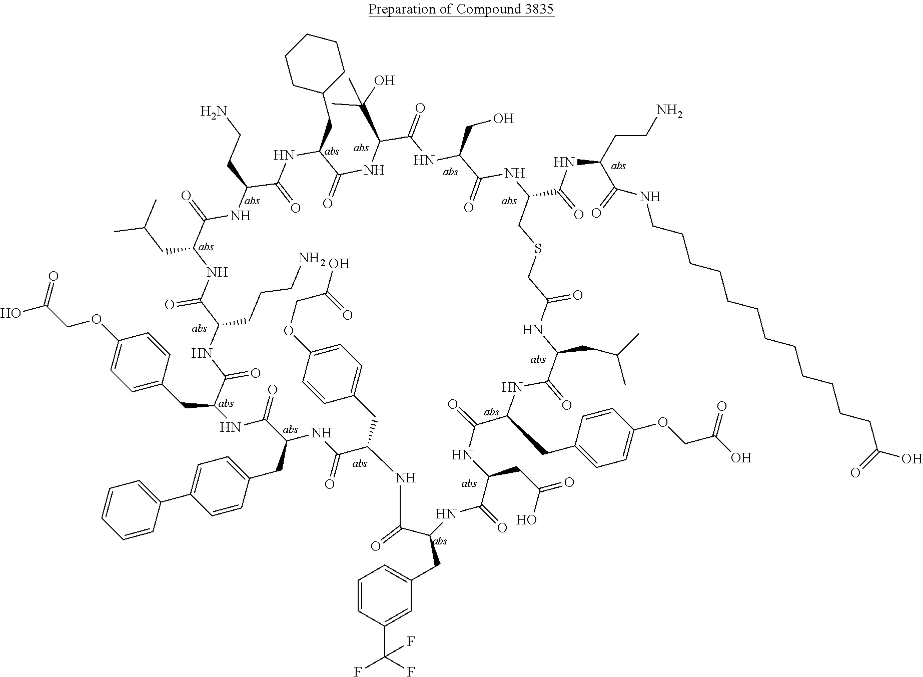 [SMILES: CC(C)C[C@@H]1NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3835 was prepared on a 50 μmol scale. The yield of the product was 38.3 mg, and its estimated purity by LCMS analysis was 95.2%. Analysis condition A: Retention time=1.73 min; ESI-MS(+) m/z [M+3H]³⁺: 829.1.

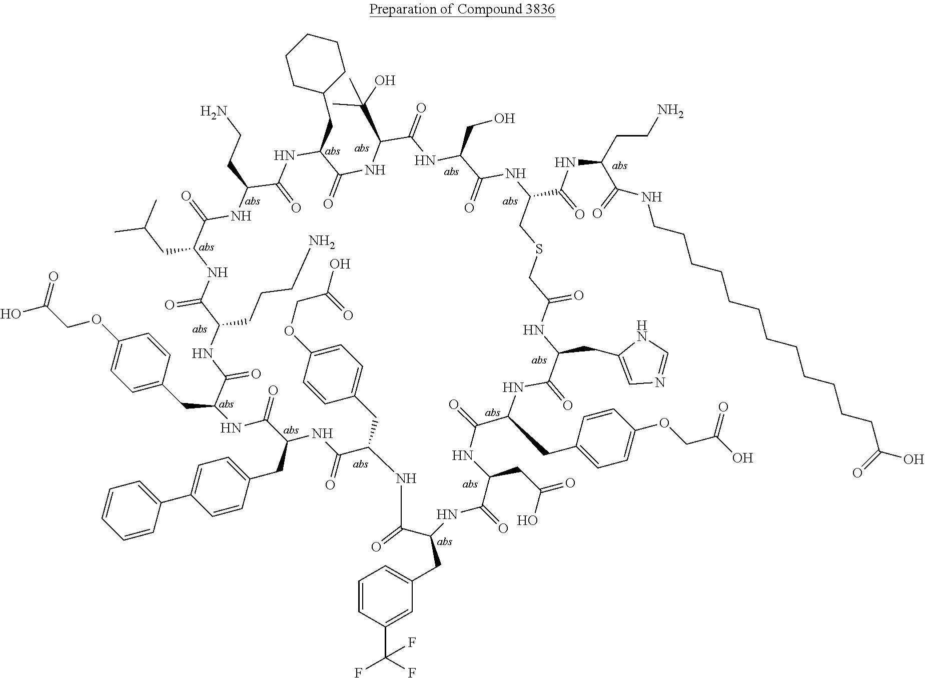 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3836 was prepared on a 50 μmol scale. The yield of the product was 7.3 mg, and its estimated purity by LCMS analysis was 100%. Analysis condition A: Retention time=1.67 min; ESI-MS(+) m/z [M+3H]³⁺: 837.

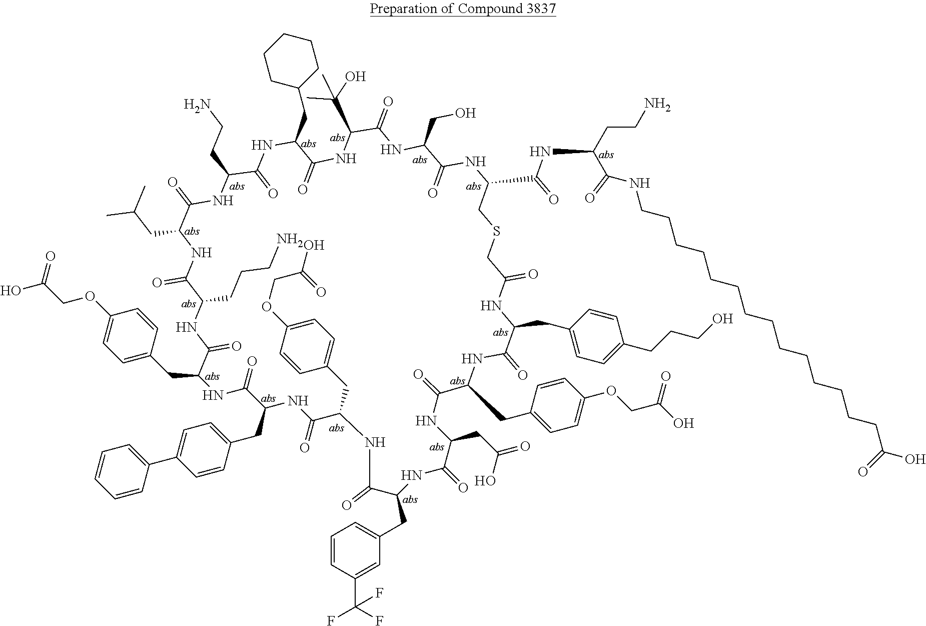 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(CCCO)cc2)NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3837 was prepared on a 50 μmol scale. The yield of the product was 43.6 mg, and its estimated purity by LCMS analysis was 90.4%. Analysis condition A: Retention time=1.49 min; ESI-MS(+) m/z [M+3H]³⁺: 865.2.

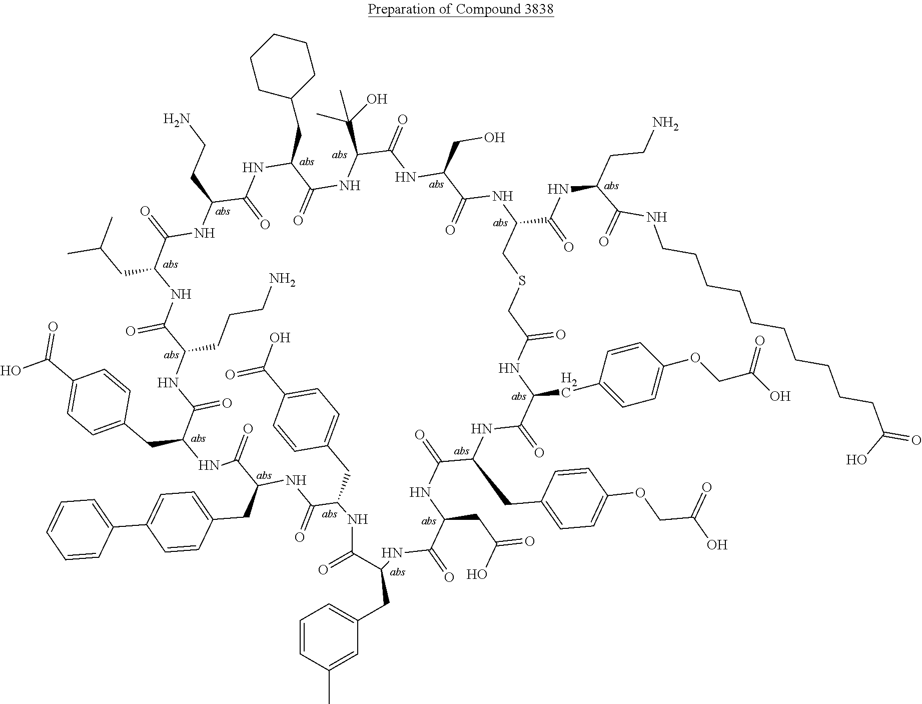 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3838 was prepared on a 50 μmol scale. The yield of the product was 19.8 mg, and its estimated purity by LCMS analysis was 100%. Analysis condition A: Retention time=1.56 min; ESI-MS(+) m/z [M+2H]²⁺: 1240.2.

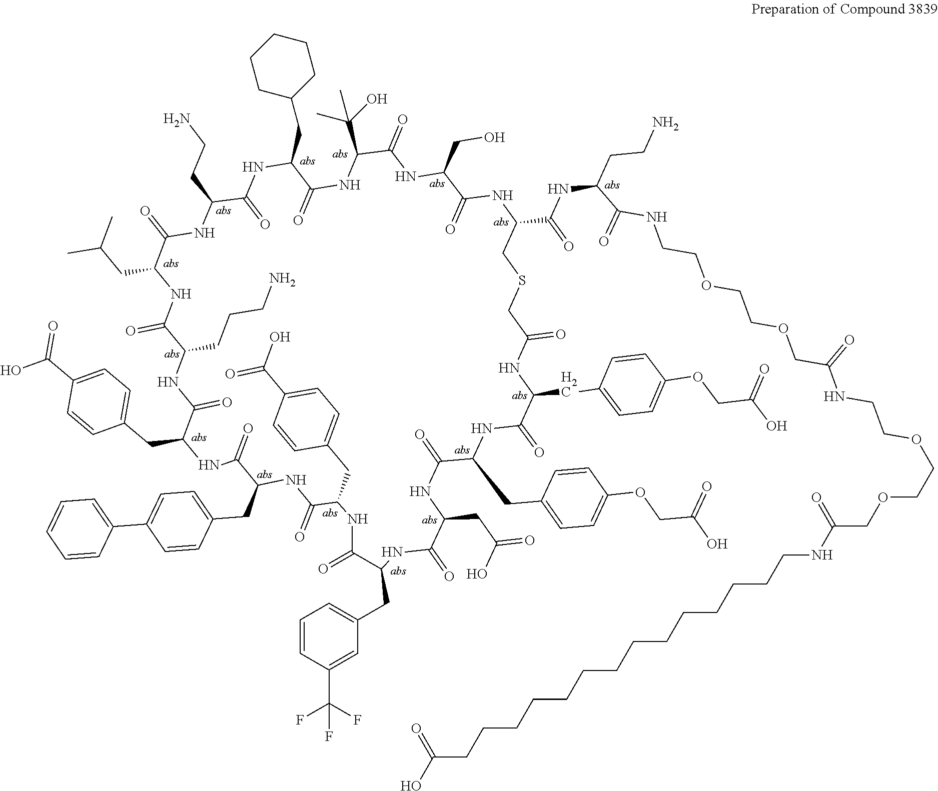 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3839 was prepared on a 5000 μmol scale. The yield of the product was 17.2 mg, and its estimated purity by LCMS analysis was 95.7%. Analysis condition A: Retention time=1.59 min; ESI-MS(+) m/z [M+2H]²⁺: 1426.3.

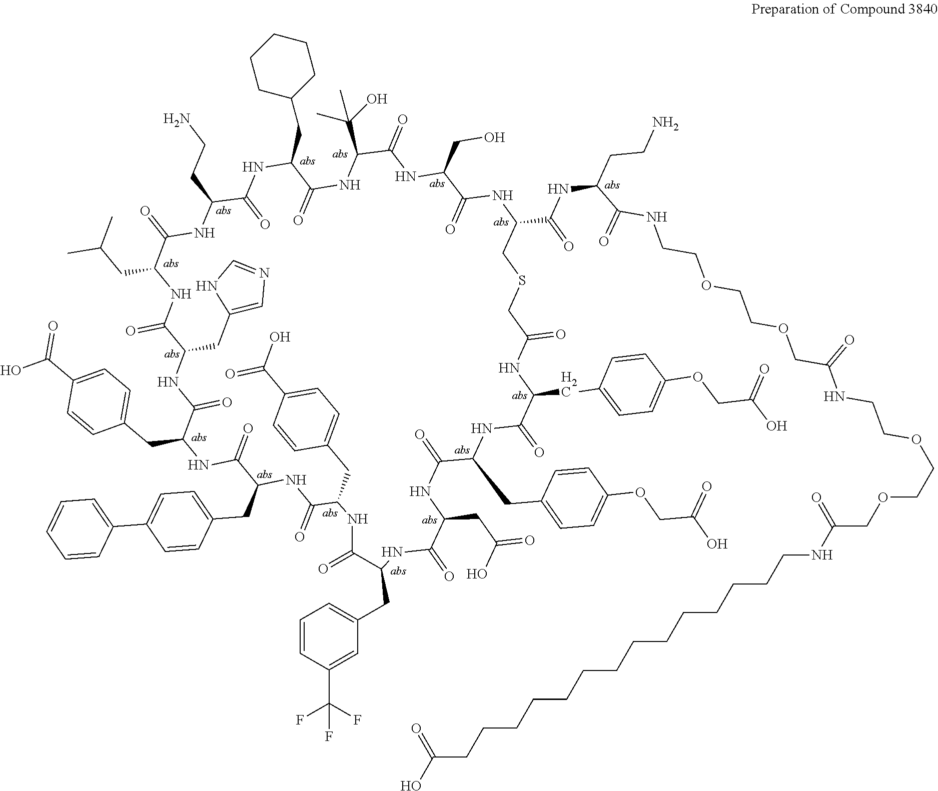 [SMILES: CC(C)C[C@H]1NC(=O)[C@H](Cc2cnc[nH]2)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3840 was prepared on a 5000 μmol scale. The yield of the product was 39.8 mg, and its estimated purity by LCMS analysis was 100%. Analysis condition A: Retention time=1.57 min; ESI-MS(+) m/z [M+2H]²⁺: 1438.2.

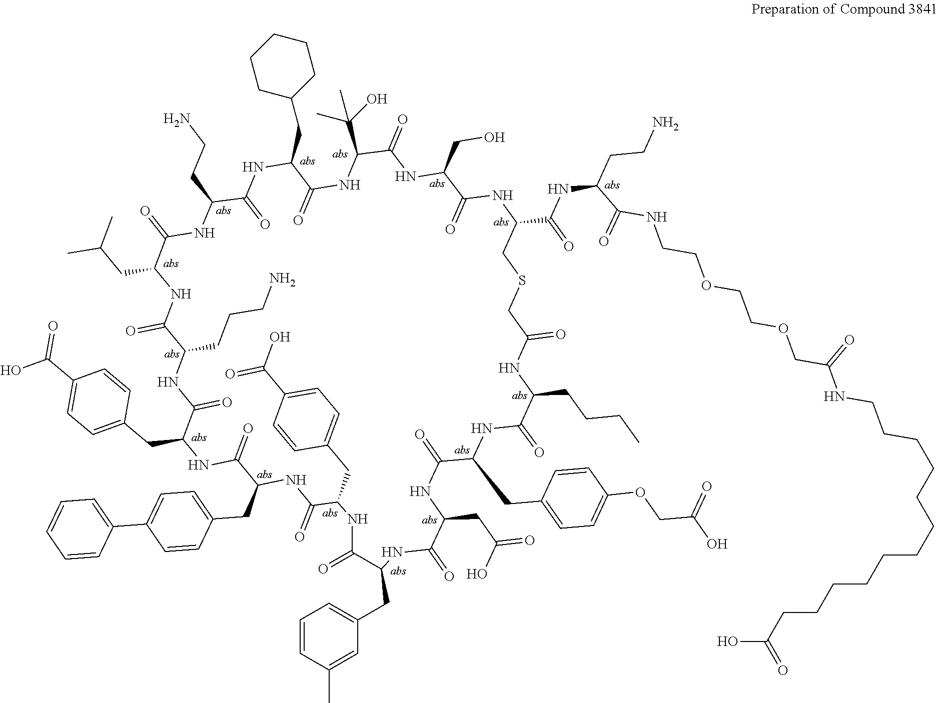 [SMILES: CCCC[C@@H]1NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3841 was prepared on a 5000 μmol scale. The yield of the product was 22.4 mg, and its estimated purity by LCMS analysis was 95.2%. Analysis condition A: Retention time=1.61 min; ESI-MS(+) m/z [M+2H]²⁺: 1259.

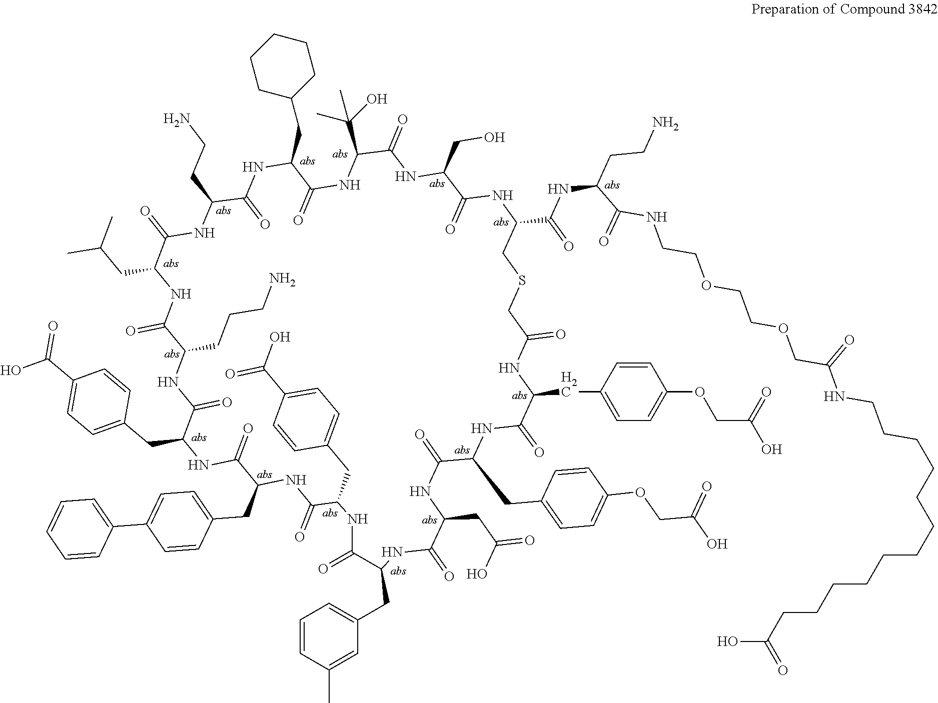 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(=O)NCCOCCOCC(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3842 was prepared on a 50 μmol scale. The yield of the product was 29.9 mg, and its estimated purity by LCMS analysis was 90.8%. Analysis condition A: Retention time=1.52 min; ESI-MS(+) m/z [M+2H]²⁺: 1313.2.

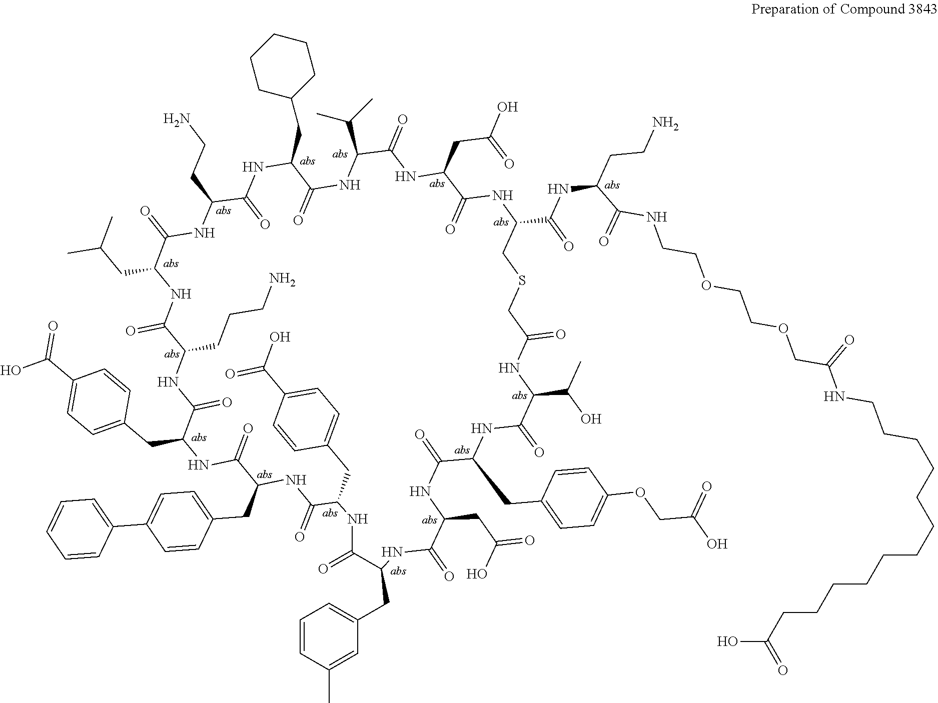 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)NCCCCCCCCCCCCC(=O)O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3843 was prepared on a 50 μmol scale. The yield of the product was 8.6 mg, and its estimated purity by LCMS analysis was 97.7%. Analysis condition A: Retention time=1.45 min; ESI-MS(+) m/z [M+2H]²⁺: 1259.2.

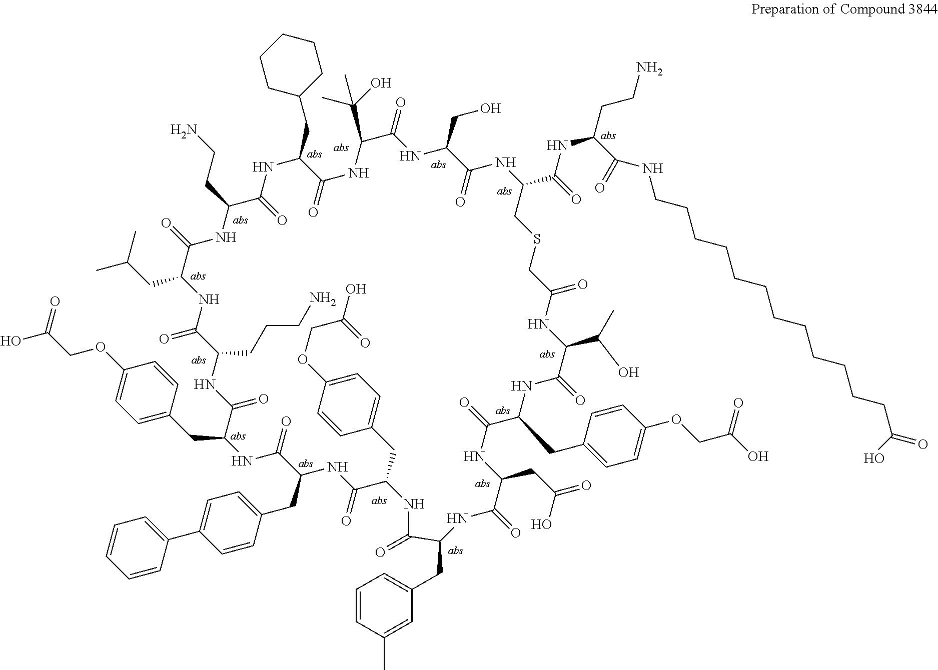 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3844 was prepared on a 50 μmol scale. The yield of the product was 13.1 mg, and its estimated purity by LCMS analysis was 96.5%. Analysis condition A: Retention time=1.53 min; ESI-MS(+) m/z [M+3H]³⁺: 807.1.

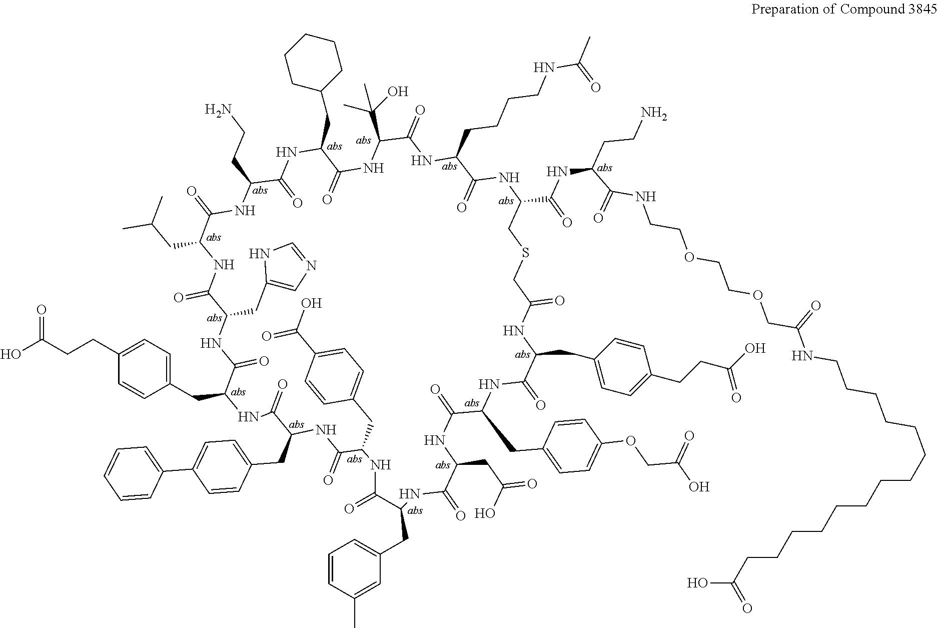 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](Cc2cnc[nH]2)NC(=O)[C@H](Cc2ccc(CCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(CCC(=O)O)cc2)NC(=O)CSC[C@@H](C(=O)N[C@@H](CCN)C(=O)NCCOCCOCC(=O)NCCCCCCCCCCCCCCC(=O)O)NC1=O]

Compound 3845 was prepared on a 50 μmol scale. The yield of the product was 37.2 mg, and its estimated purity by LCMS analysis was 96.7%. Analysis condition B: Retention time=1.83 min; ESI-MS(+) m/z [M+2H]²⁺: 1394.9.

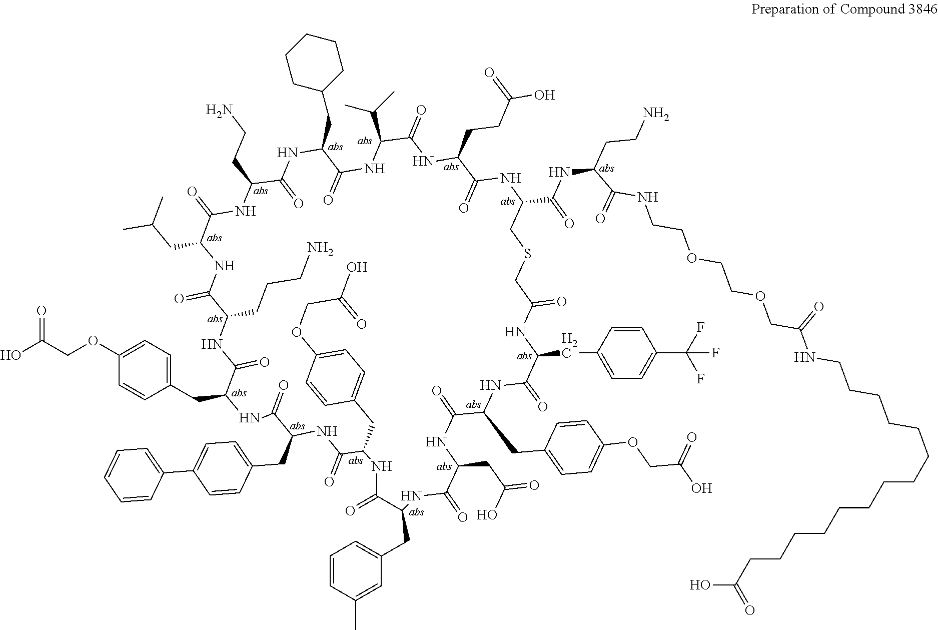 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(F)(F)F)cc3)NC(=O)CSC[C@@H](C(=O)N[C@@H](CCN)C(=O)NCCOCCOCC(=O)NCCCCCCCCCCCCCCC(=O)O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3846 was prepared on a 50 μmol scale. The yield of the product was 32.5 mg, and its estimated purity by LCMS analysis was 95.6%. Analysis condition A: Retention time=1.62 min; ESI-MS(+) m/z [M+2H]²⁺: 1353.1.

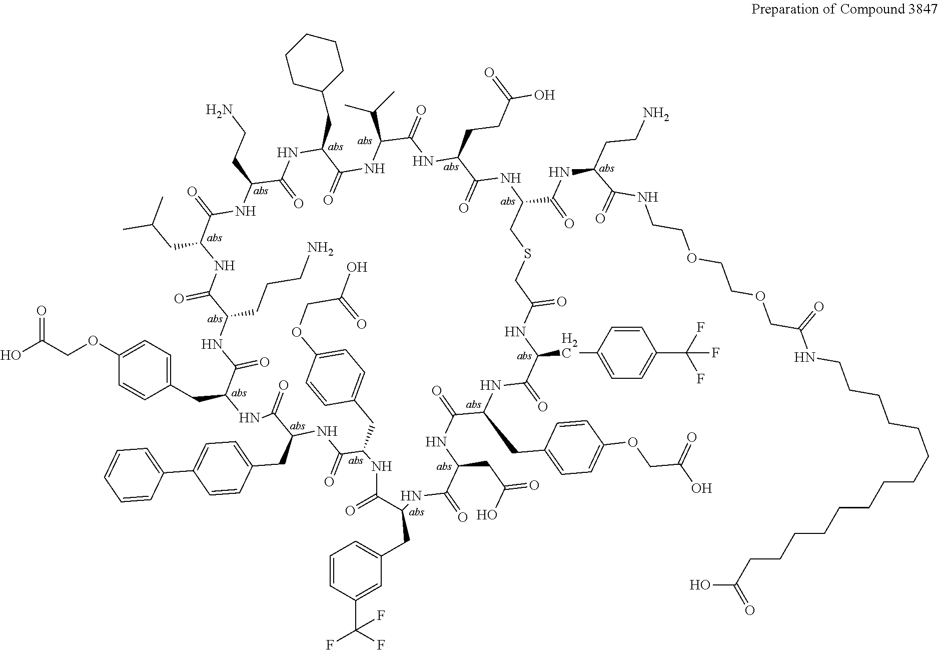 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(C(F)(F)F)cc2)NC(=O)CSC[C@@H](C(=O)N[C@@H](CCN)C(=O)NCCOCCOCC(=O)NCCCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3847 was prepared on a 50 μmol scale. The yield of the product was 14.7 mg, and its estimated purity by LCMS analysis was 100%. Analysis condition A: Retention time=1.66 min; ESI-MS(+) m/z [M+2H]²⁺: 1380.

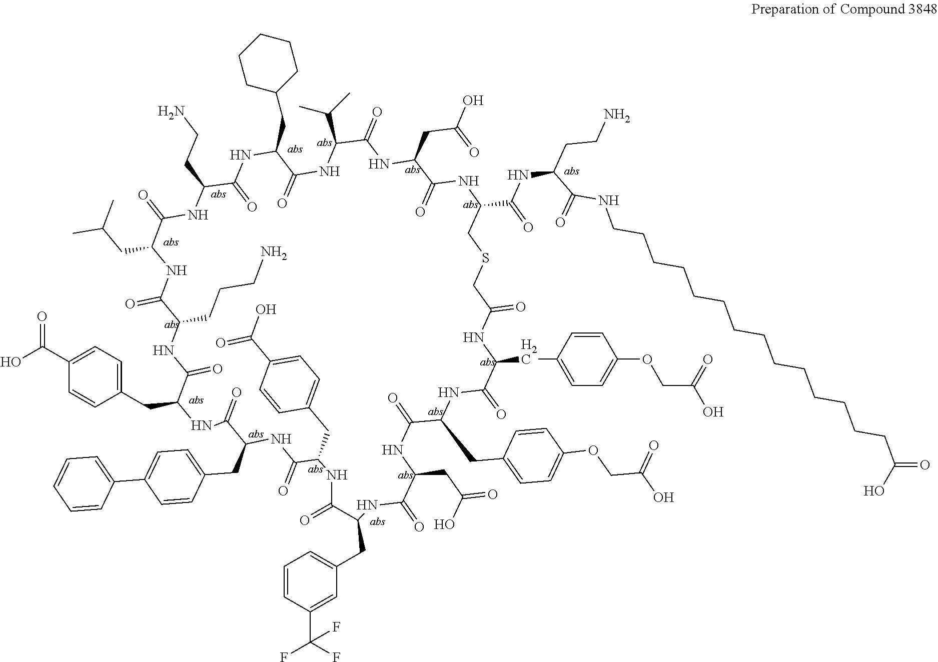 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC1=O]

Compound 3848 was prepared on a 50 μmol scale. The yield of the product was 11.4 mg, and its estimated purity by LCMS analysis was 95.4%. Analysis condition B: Retention time=1.86 min; ESI-MS(+) m/z [M+2H]²⁺: 1287.

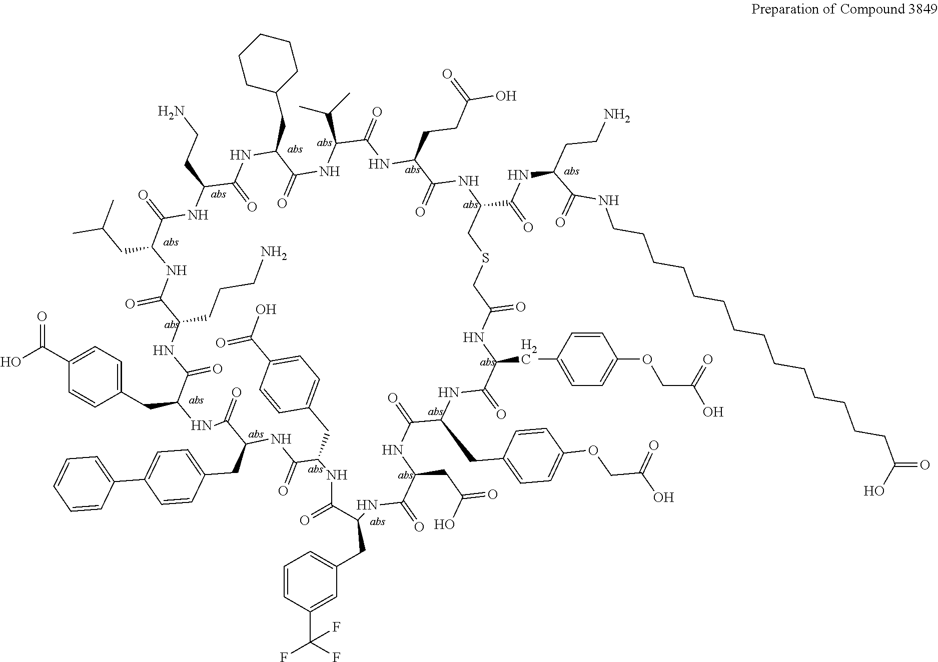 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3849 was prepared on a 50 μmol scale. The yield of the product was 19.1 mg, and its estimated purity by LCMS analysis was 100%. Analysis condition A: Retention time=1.49 min; ESI-MS(+) m/z [M+2H]²⁺: 1295.

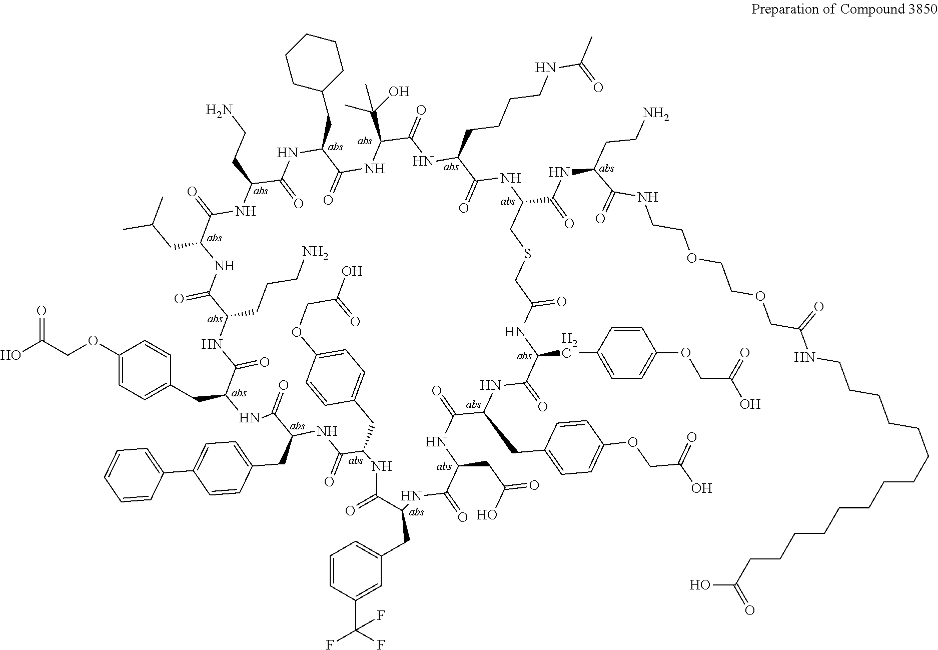 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCCCCCC(=O)O)NC1=O]

Compound 3850 was prepared on a 50 μmol scale. The yield of the product was 44.9 mg, and its estimated purity by LCMS analysis was 100%. Analysis condition A: Retention time=1.56 min; ESI-MS(+) m/z [M+2H]²⁺: 1425.3.

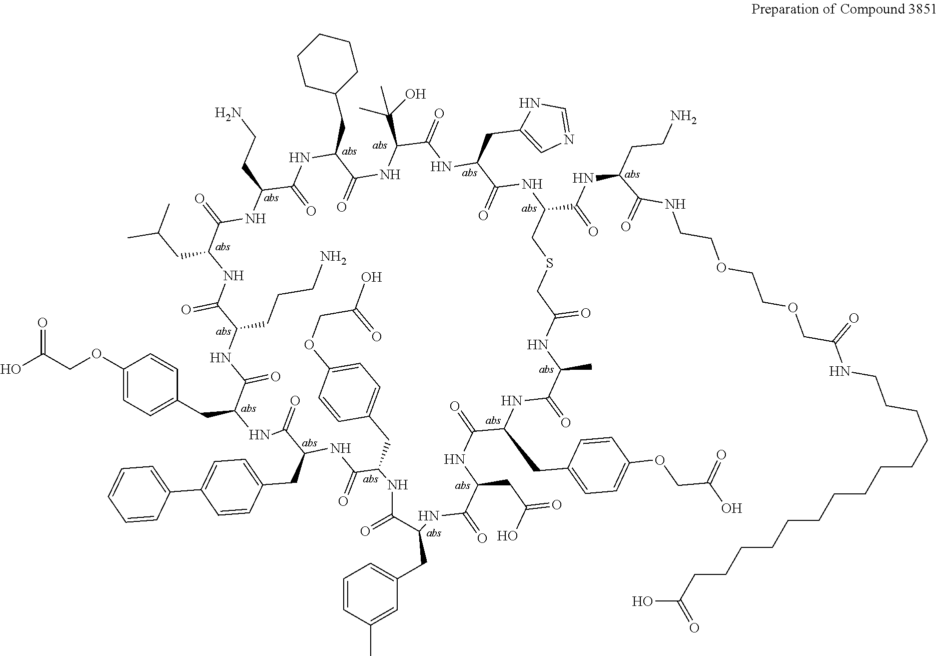 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3851 was prepared on a 50 μmol scale. The yield of the product was 28.3 mg, and its estimated purity by LCMS analysis was 91.9%. Analysis condition A: Retention time-1.67 min; ESI-MS(+) m/z [M+2H]²⁺: 1307.2.

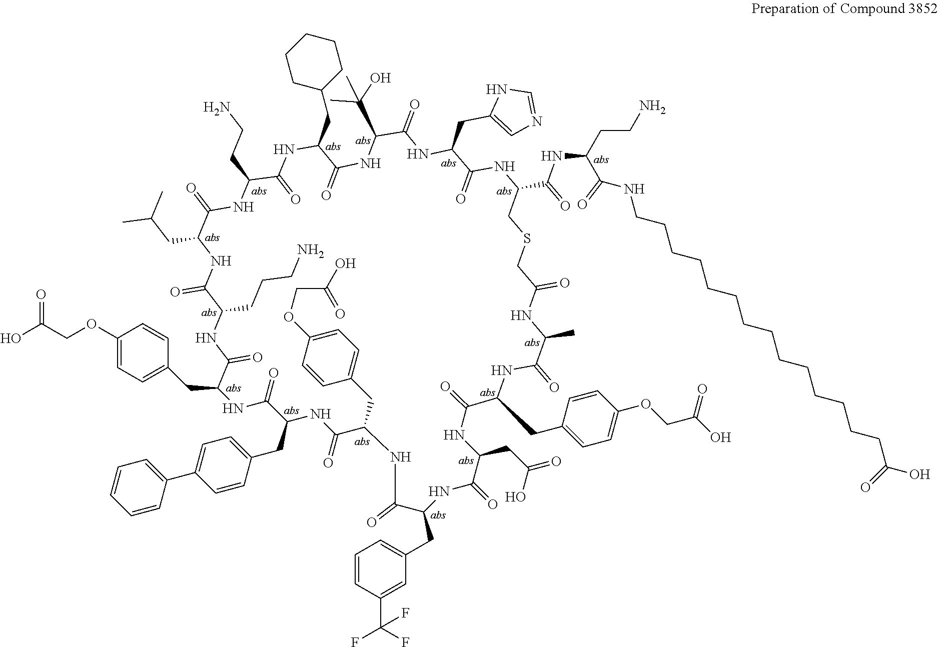 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCCCCCCCCCCCCCC(=O)O)NC(=O)[C@H](Cc2cnc[nH]2)NC(=O)[C@H](C(C)(C)O)NC(=O)[C@H](CC2CCCCC2)NC(=O)[C@H](CCN)NC1=O]

Compound 3852 was prepared on a 50 μmol scale. The yield of the product was 30.8 mg, and its estimated purity by LCMS analysis was 100%. Analysis condition A: Retention time=1.73 min; ESI-MS(+) m/z [M+2H]²⁺: 1261.1.

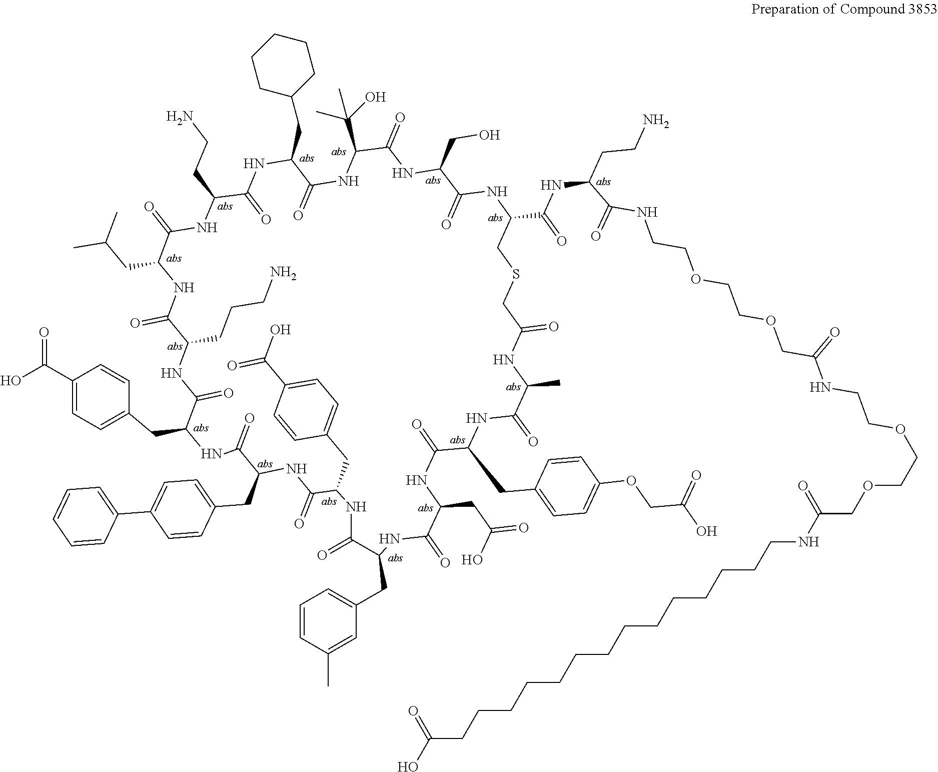 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3853 was prepared on a 50 μmol scale. The yield of the product was 15.3 mg, and its estimated purity by LCMS analysis was 100%. Analysis condition A: Retention time=1.61 min; ESI-MS(+) m/z [M+2H]²⁺: 1324.4.

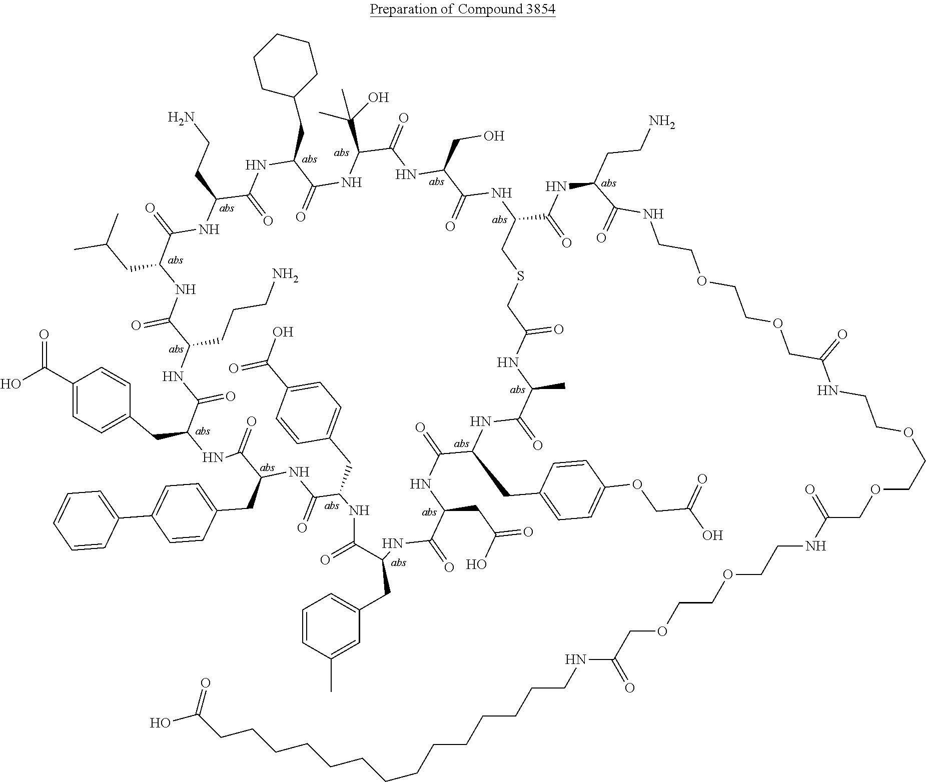 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3854 was prepared on a 50 μmol scale. The yield of the product was 18.8 mg, and its estimated purity by LCMS analysis was 96.1%. Analysis condition A: Retention time=1.6 min; ESI-MS(+) m/z [M+2H]²⁺: 1397.

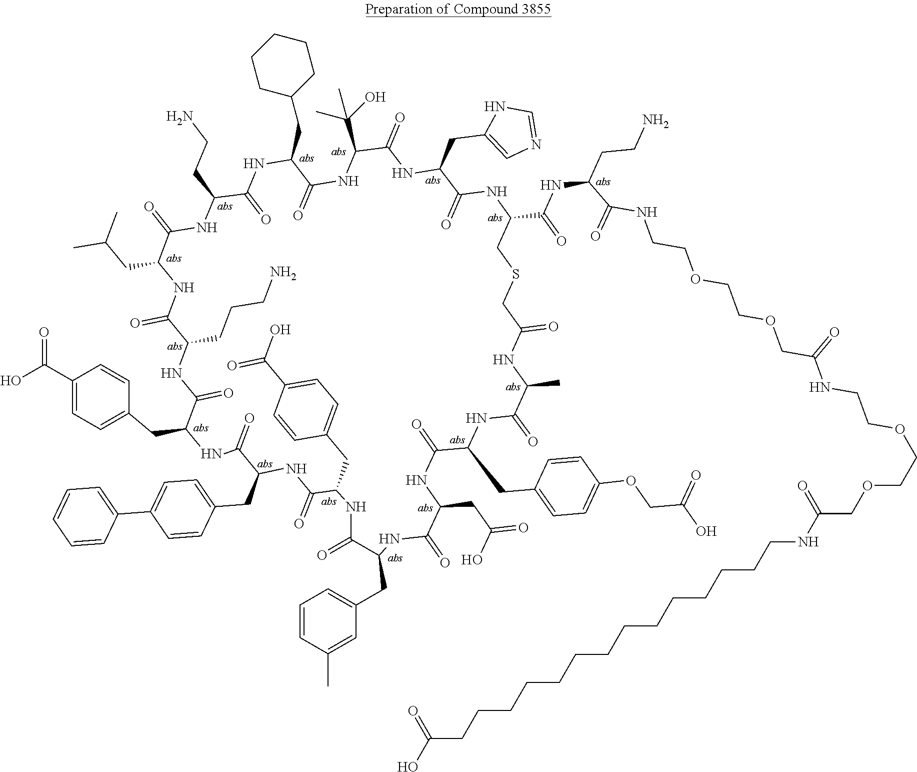 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3855 was prepared on a 50 μmol scale. The yield of the product was 24.6 mg, and its estimated purity by LCMS analysis was 89.1%. Analysis condition A: Retention time=1.6 min; ESI-MS(+) m/z [M+3H]³⁺: 900.2.

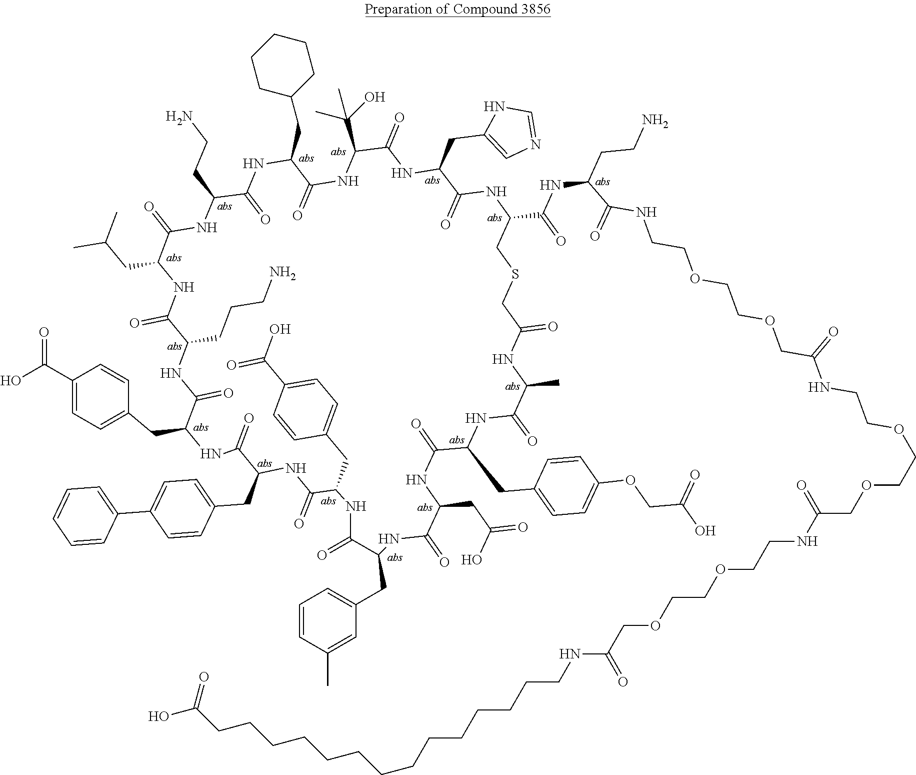 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCOCCOCC(=O)NCCOCCOCC(=O)NCCCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3856 was prepared on a 50 μmol scale. The yield of the product was 10.2 mg, and its estimated purity by LCMS analysis was 95.1%. Analysis condition B: Retention time=1.78 min; ESI-MS(+) m/z [M+2H]²⁺: 1422.1.

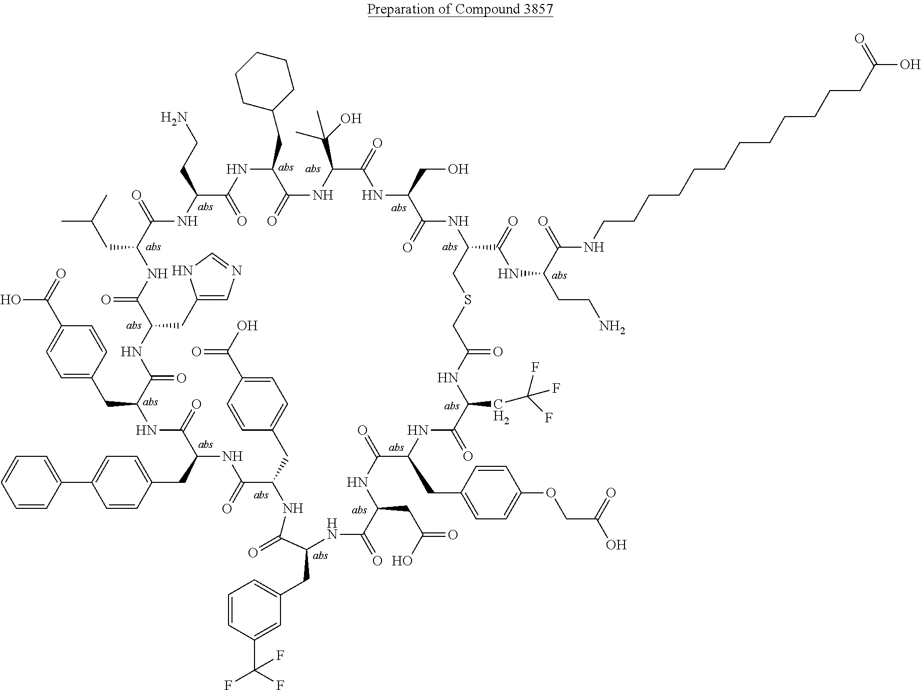 [SMILES: CC(C)C[C@H]1NC(=O)[C@H](Cc2cnc[nH]2)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CC(F)(F)F)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3857 was prepared on a 50 μmol scale. The yield of the product was 12.8 mg, and its estimated purity by LCMS analysis was 100%. Analysis condition B: Retention time=1.86 min; ESI-MS(+) m/z [M+2H]²⁺: 1238.2.

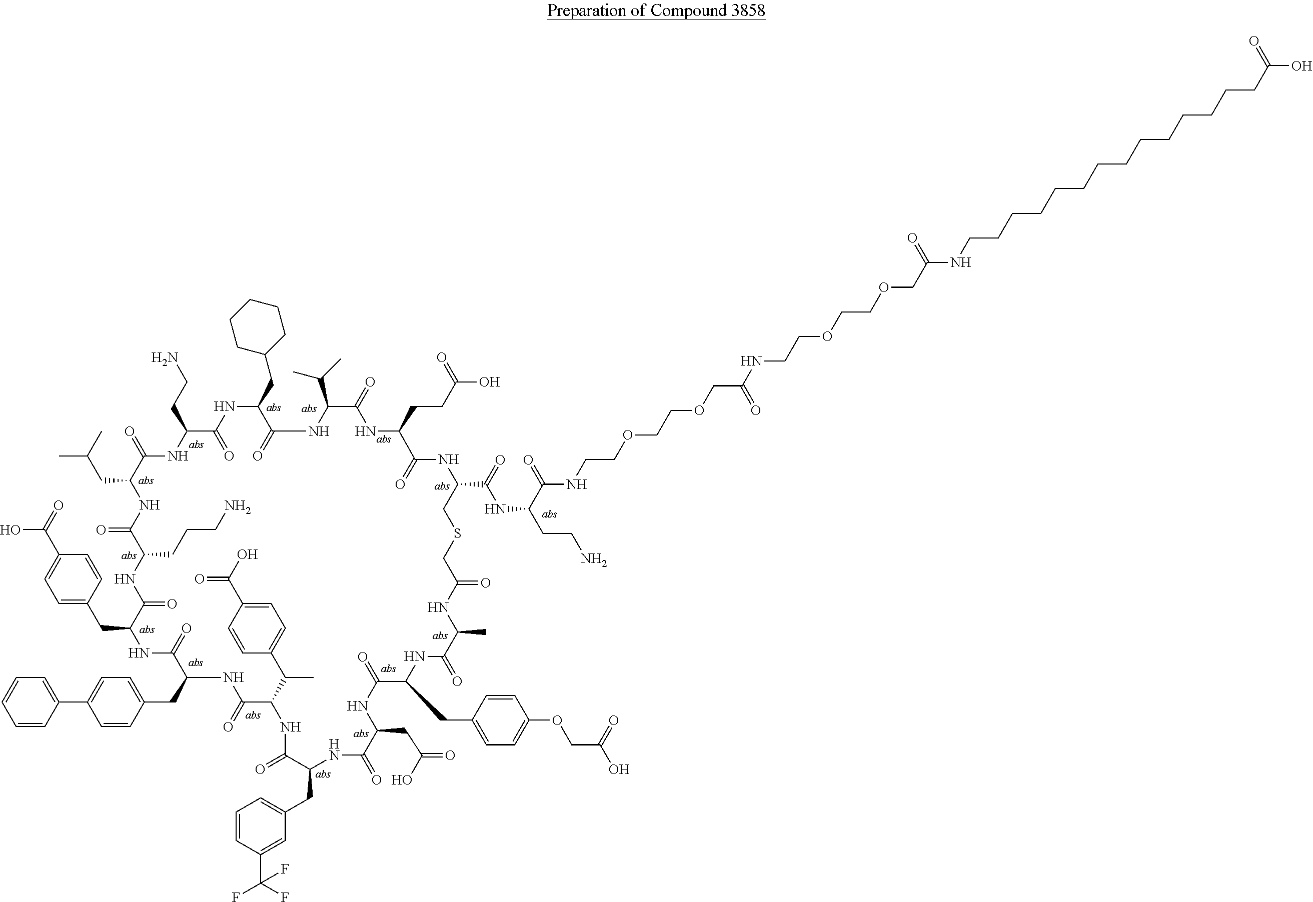 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](C(C)c2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCOCCOCC(=O)NCCOCCOCC(=O)NCCCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3858 was prepared on a 50 μmol scale. The yield of the product was 8.8 mg, and its estimated purity by LCMS analysis was 92.6%. Analysis condition B: Retention time=1.97 min; ESI-MS(+) m/z [M+3H]³⁺: 910.

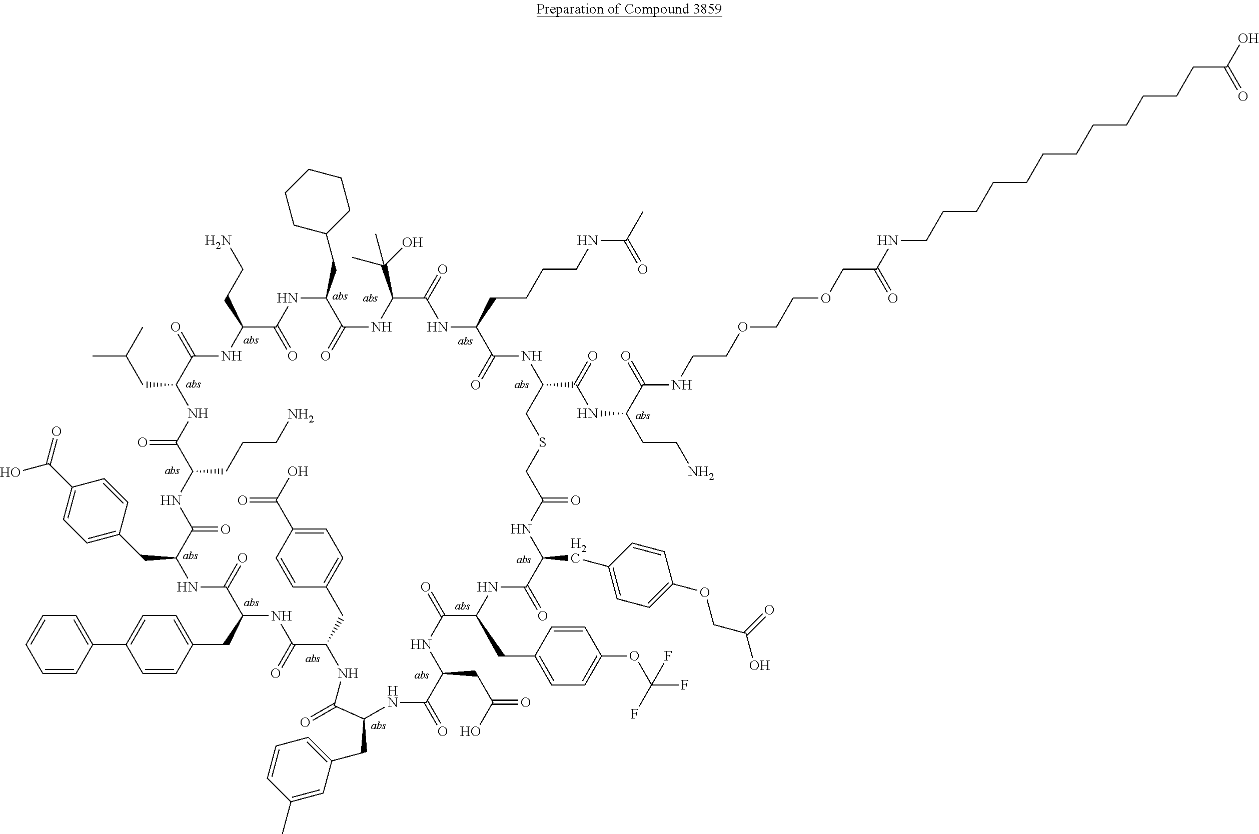 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OC(F)(F)F)cc2)NC(=O)[C@H](Cc2ccc(OCC(=O)O)cc2)NC(=O)CSC[C@@H](C(=O)N[C@@H](CCN)C(=O)NCCOCCOCC(=O)NCCCCCCCCCCCCC(=O)O)NC1=O]

Compound 3859 was prepared on a 50 μmol scale. The yield of the product was 19.3 mg, and its estimated purity by LCMS analysis was 95.1%. Analysis condition B: Retention time=2.05 min; ESI-MS(+) m/z [M+2H]²⁺: 1360.

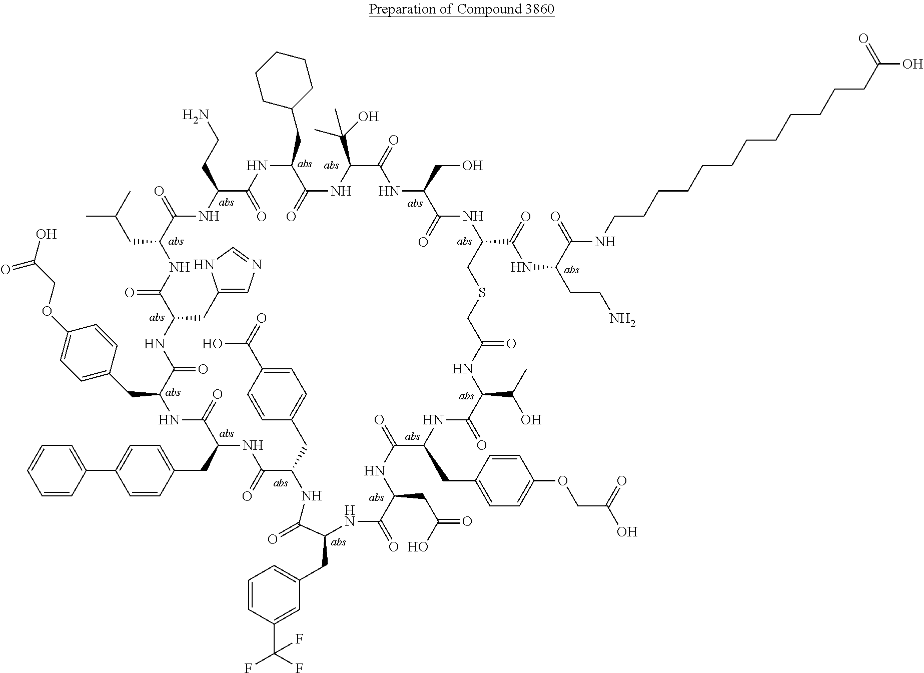 [SMILES: CC(C)C[C@H]1NC(=O)[C@H](Cc2cnc[nH]2)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3860 was prepared on a 50 μmol scale. The yield of the product was 21.4 mg, and its estimated purity by LCMS analysis was 96.9%. Analysis condition B: Retention time=1.58 min; ESI-MS(+) m/z [M+2H]²⁺: 1241.

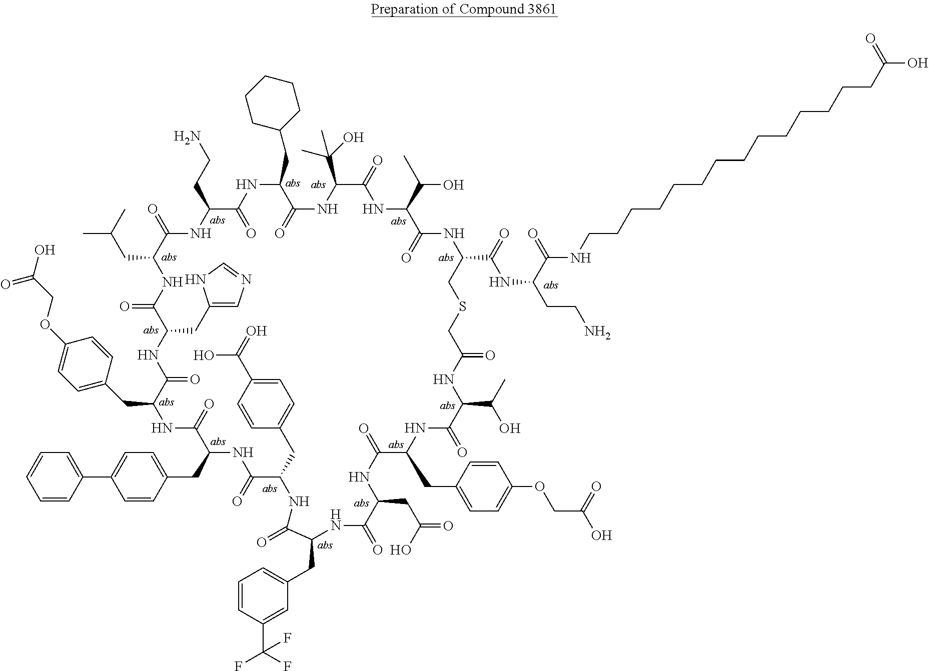 [SMILES: CC(C)C[C@H]1NC(=O)[C@H](Cc2cnc[nH]2)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCCCCCCCCCCCCCC(=O)O)NC(=O)[C@H](C(C)O)NC(=O)[C@H](C(C)(C)O)NC(=O)[C@H](CC2CCCCC2)NC(=O)[C@H](CCN)NC1=O]

Compound 3861 was prepared on a 50 μmol scale. The yield of the product was 15.2 mg, and its estimated purity by LCMS analysis was 98%. Analysis condition A: Retention time=1.72 min; ESI-MS(+) m/z [M+2H]²⁺: 1254.9.

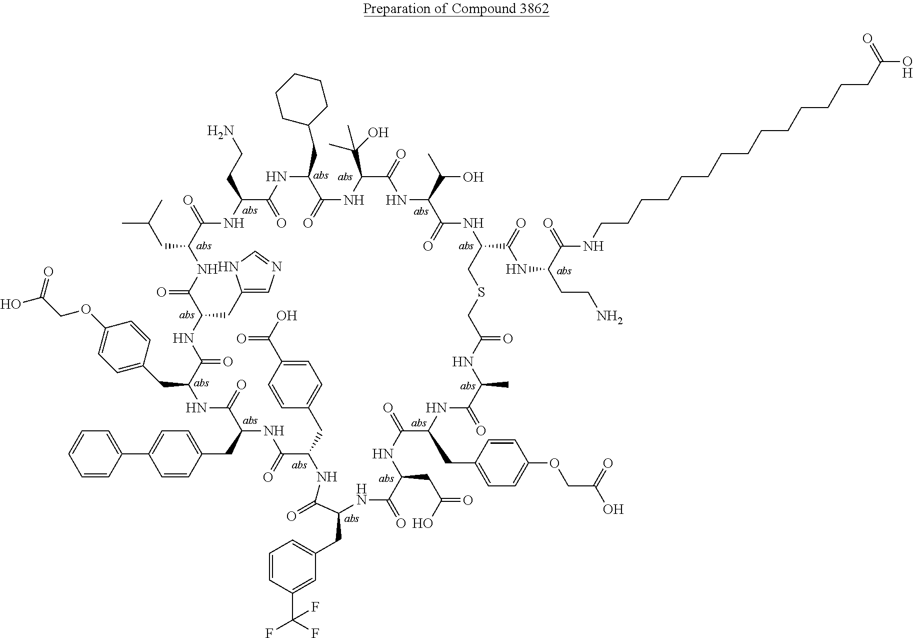 [SMILES: CC(C)C[C@H]1NC(=O)[C@H](Cc2cnc[nH]2)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C)NC(=O)CSC[C@@H](C(=O)N[C@@H](CCN)C(=O)NCCCCCCCCCCCCCCC(=O)O)NC(=O)[C@H](C(C)O)NC(=O)[C@H](C(C)(C)O)NC(=O)[C@H](CC2CCCCC2)NC(=O)[C@H](CCN)NC1=O]

Compound 3862 was prepared on a 50 μmol scale. The yield of the product was 32.6 mg, and its estimated purity by LCMS analysis was 93.9%. Analysis condition A: Retention time=1.67 min; ESI-MS(+) m/z [M+2H]²⁺: 1240.1.

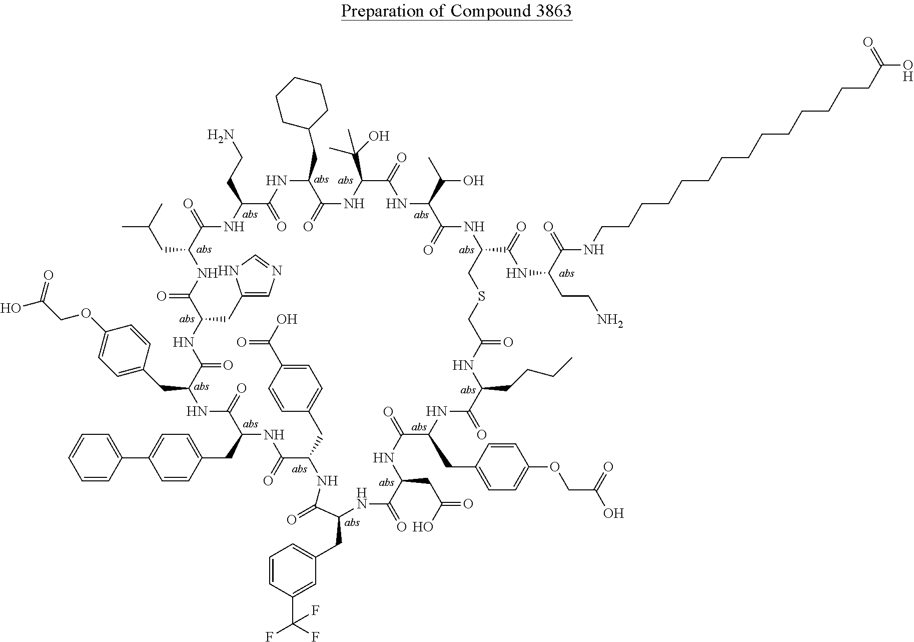 [SMILES: CCCC[C@@H]1NC(=O)CSC[C@@H](C(=O)N[C@@H](CCN)C(=O)NCCCCCCCCCCCCCCC(=O)O)NC(=O)[C@H](C(C)O)NC(=O)[C@H](C(C)(C)O)NC(=O)[C@H](CC2CCCCC2)NC(=O)[C@H](CCN)NC(=O)[C@@H](CC(C)C)NC(=O)[C@H](Cc2cnc[nH]2)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3863 was prepared on a 50 μmol scale. The yield of the product was 34.1 mg, and its estimated purity by LCMS analysis was 100%. Analysis condition B: Retention time=1.99 min; ESI-MS(+) m/z [M+2H]²⁺: 1261.1.

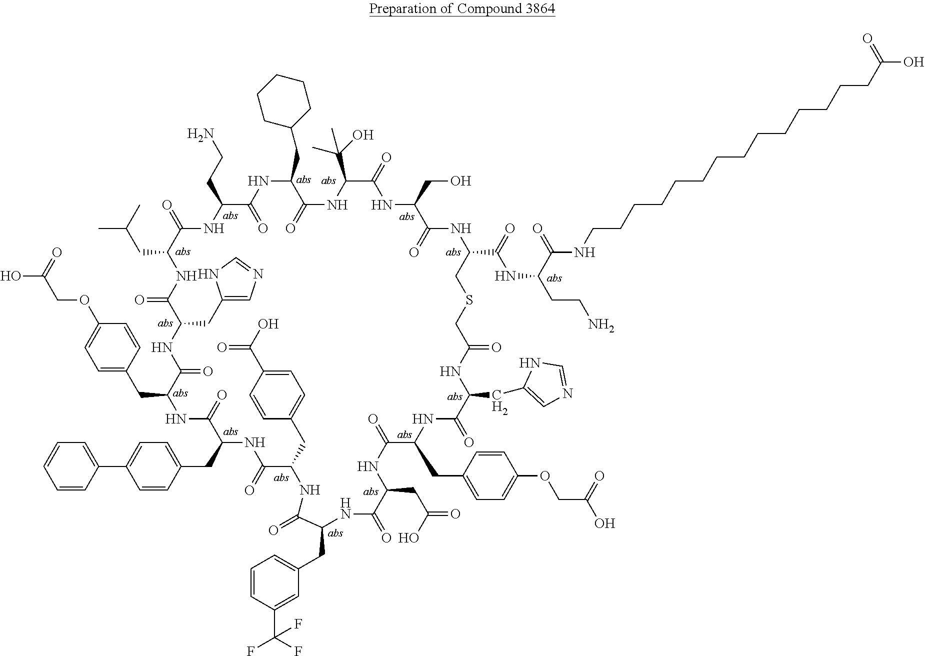 [SMILES: CC(C)C[C@H]1NC(=O)[C@H](Cc2cnc[nH]2)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC1=O]

Compound 3864 was prepared on a 50 μmol scale. The yield of the product was 19.6 mg, and its estimated purity by LCMS analysis was 95.7%. Analysis condition B: Retention time=1.69 min; ESI-MS(+) m/z [M+2H]²⁺: 1269.6.

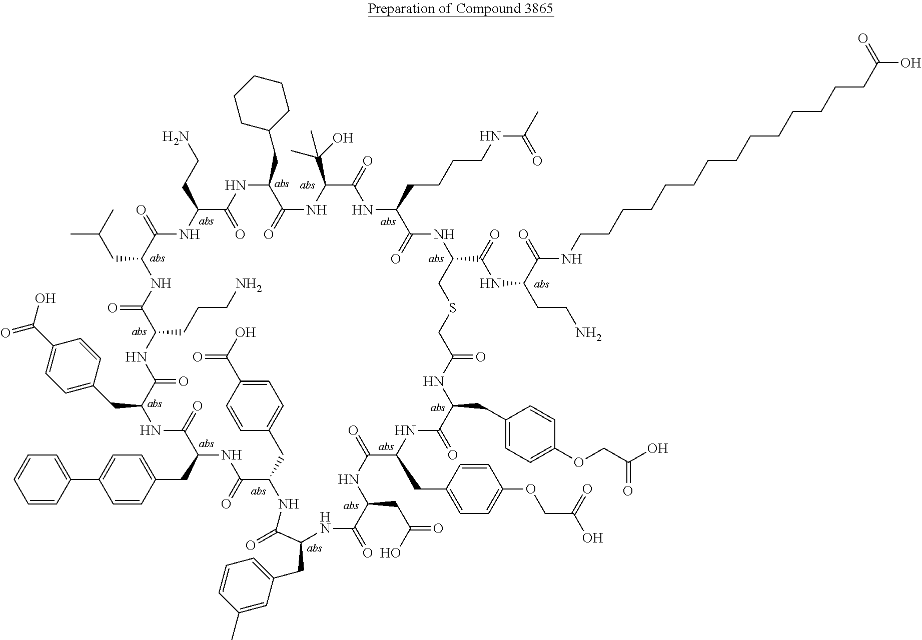 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC1=O]

Compound 3865 was prepared on a 50 μmol scale. The yield of the product was 14.8 mg, and its estimated purity by LCMS analysis was 96%. Analysis condition B: Retention time=1.47 min; ESI-MS(+) m/z [M+2H]²⁺: 1296.2.

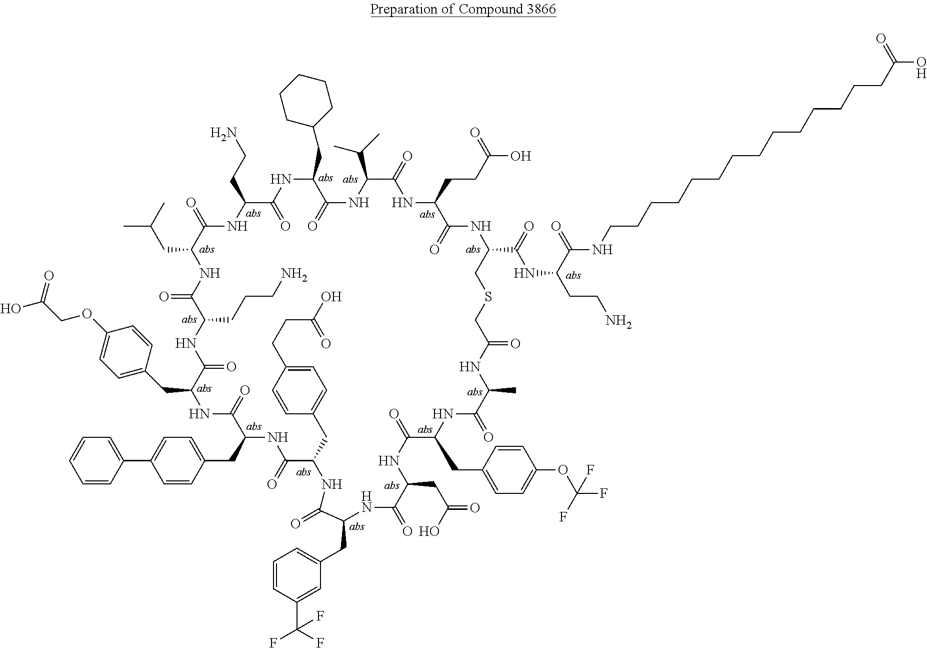 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(CCC(=O)O)cc2)NC(=O)[C@H](Cc2cccc(C(F)(F)F)c2)NC(=O)[C@H](CC(=O)O)NC(=O)[C@H](Cc2ccc(OC(F)(F)F)cc2)NC(=O)[C@H](C)NC(=O)CSC[C@@H](C(=O)N[C@@H](CCN)C(=O)NCCCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3866 was prepared on a 50 μmol scale. The yield of the product was 2 mg, and its estimated purity by LCMS analysis was 100%. Analysis condition B: Retention time=2.11 min; ESI-MS(+) m/z [M+2H]²⁺: 1255.1.

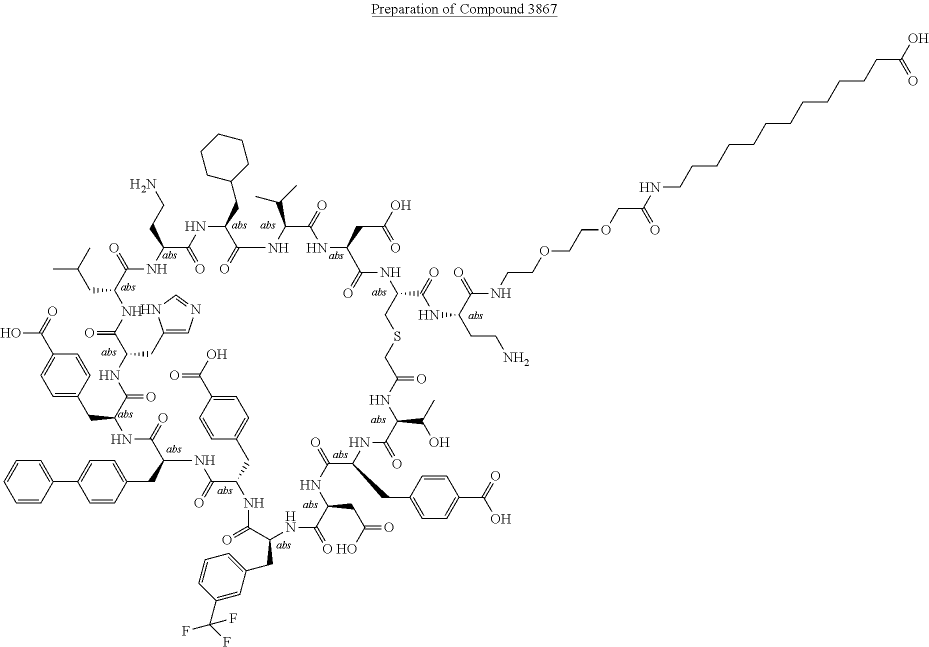 [SMILES: CC(C)C[C@H]1NC(=O)[C@H](Cc2cnc[nH]2)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](C(C)O)NC(=O)CSC[C@@H](C(=O)N[C@@H](CCN)C(=O)NCCOCCOCC(=O)NCCCCCCCCCCCCC(=O)O)NC(=O)[C@H](CC(=O)O)NC(=O)[C@H](C(C)C)NC(=O)[C@H](CC2CCCCC2)NC(=O)[C@H](CCN)NC1=O]

Compound 3867 was prepared on a 50 μmol scale. The yield of the product was 59 mg, and its estimated purity by LCMS analysis was 90.4%. Analysis condition B: Retention time=1.52 min; ESI-MS(+) m/z [M+2H]²⁺: 1282.4.

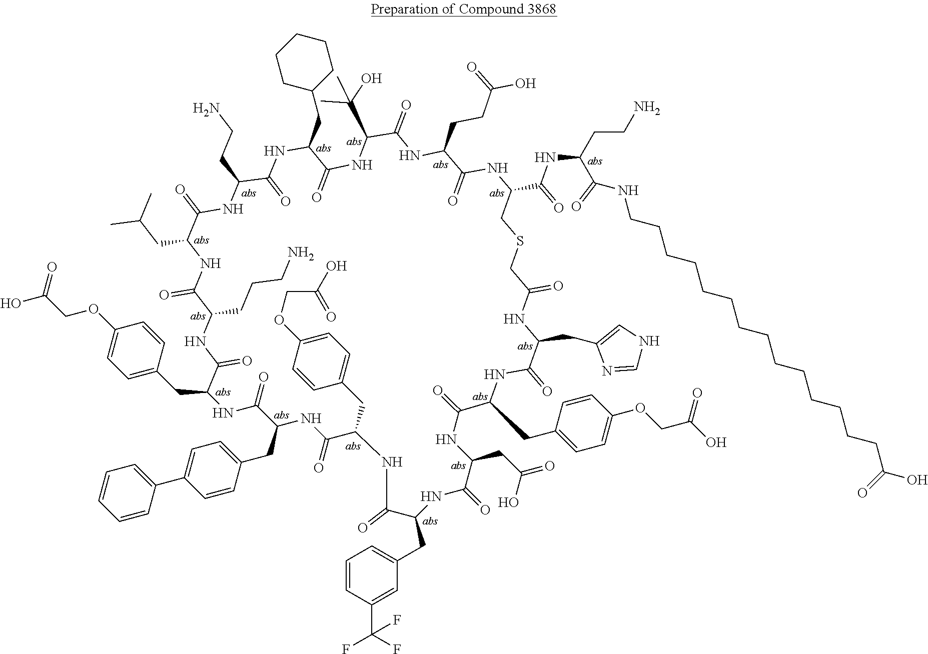 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2c[nH]cn2)NC(=O)CSC[C@@H](C(=O)N[C@@H](CCN)C(=O)NCCCCCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Compound 3868 was prepared on a 50 μmol scale. The yield of the product was 1.8 mg, and its estimated purity by LCMS analysis was 93.4%. Analysis condition B: Retention time=1.82 min; ESI-MS(+) m/z [M+3H]³⁺: 861.7.

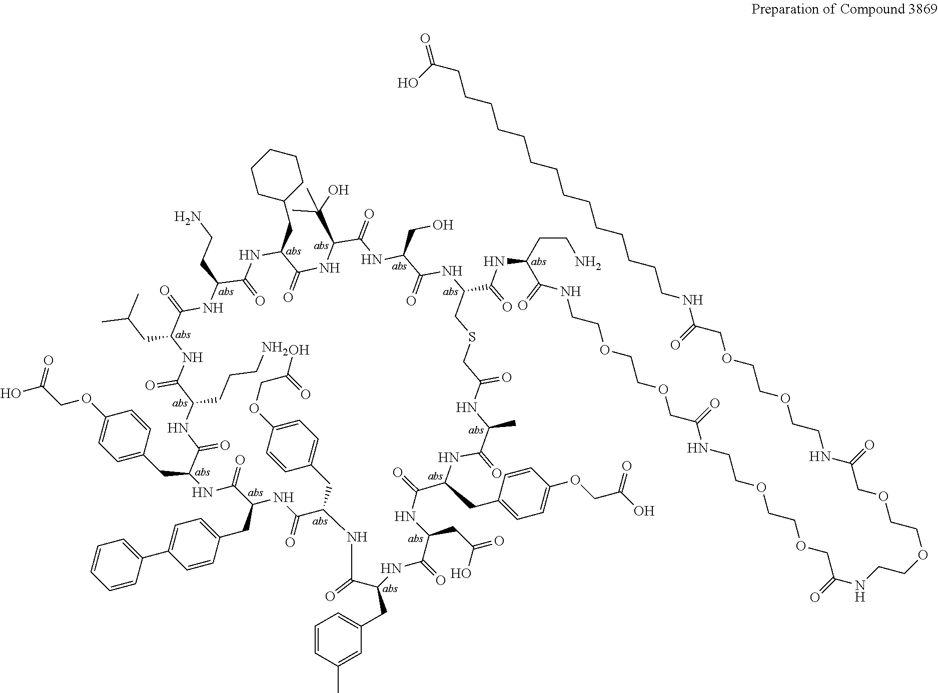 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCOCCOCC(=O)NCCOCCOCC(=O)NCCOCCOCC(=O)NCCCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3869 was prepared on a 50 μmol scale. The yield of the product was 5.7 mg, and its estimated purity by LCMS analysis was 95.4%. Analysis condition A: Retention time-1.65 min; ESI-MS(+) m/z [M+3H]³⁺: 1000.1.

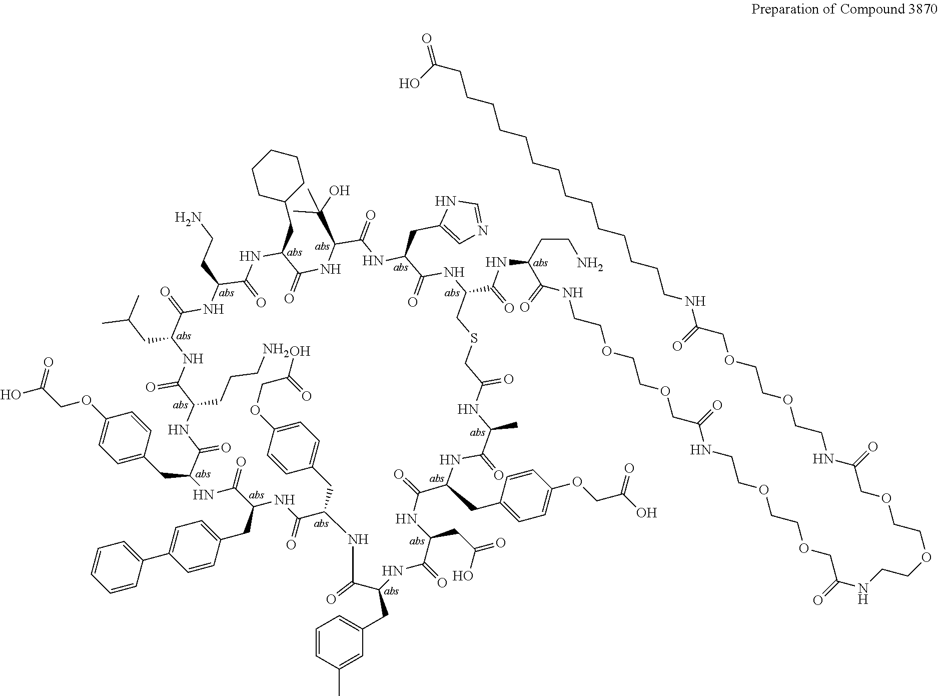 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCOCCOCC(=O)NCCOCCOCC(=O)NCCOCCOCC(=O)NCCCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3870 was prepared on a 50 μmol scale. The yield of the product was 12.3 mg, and its estimated purity by LCMS analysis was 95.3%. Analysis condition A: Retention time=1.63 min; ESI-MS(+) m/z [M+2H]²⁺: 1524.2.

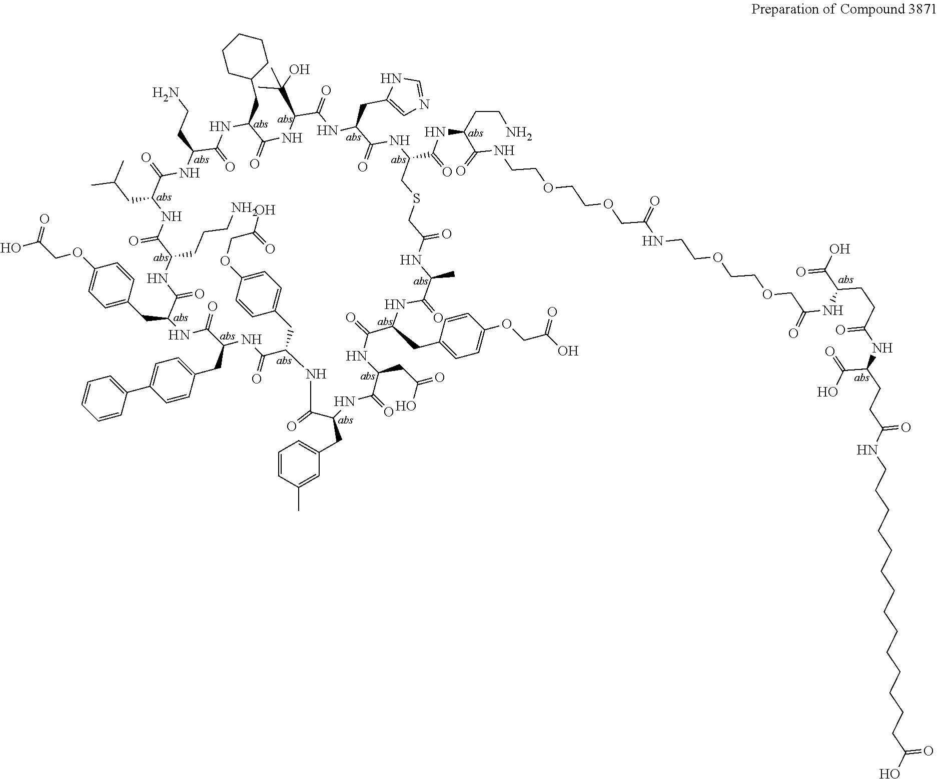 [SMILES: Cc1cccc(C[C@@H]2NC(=O)[C@H](CC(=O)O)NC(=O)[C@H](Cc3ccc(OCC(=O)O)cc3)NC(=O)[C@H](C)NC(=O)CSC[C@@H](C(=O)N[C@@H](CCN)C(=O)NCCOCCOCC(=O)NCCOCCOCC(=O)N[C@@H](CCC(=O)N[C@@H](CCC(=O)NCCCCCCCCCCCCCCC(=O)O)C(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3871 was prepared on a 50 μmol scale. The yield of the product was 8.5 mg, and its estimated purity by LCMS analysis was 97.6%. Analysis condition B: Retention time=1.77 min; ESI-MS(+) m/z [M+2H]²⁺:1523.1.

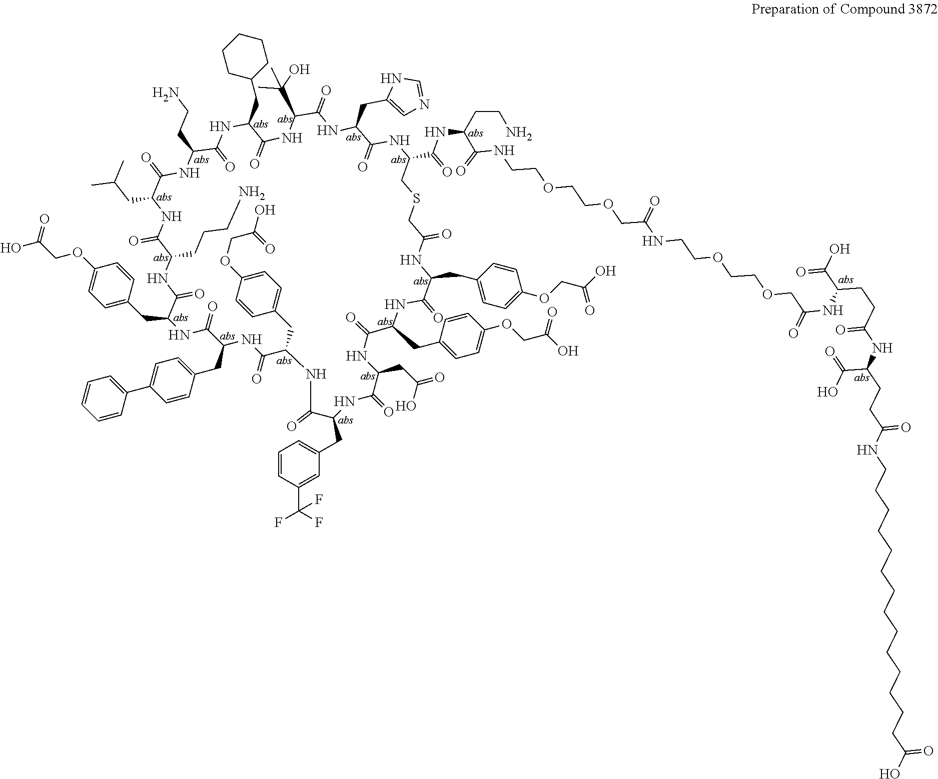 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)NCCOCCOCC(=O)N[C@@H](CCC(=O)N[C@@H](CCC(=O)NCCCCCCCCCCCCCCC(=O)O)C(=O)O)C(=O)O)NC(=O)[C@H](Cc2cnc[nH]2)NC(=O)[C@H](C(C)(C)O)NC(=O)[C@H](CC2CCCCC2)NC(=O)[C@H](CCN)NC1=O]

Compound 3872 was prepared on a 50 μmol scale. The yield of the product was 17.6 mg, and its estimated purity by LCMS analysis was 96.9%. Analysis condition A: Retention time=1.52 min; ESI-MS(+) m/z [M+2H]²⁺: 1610.6.

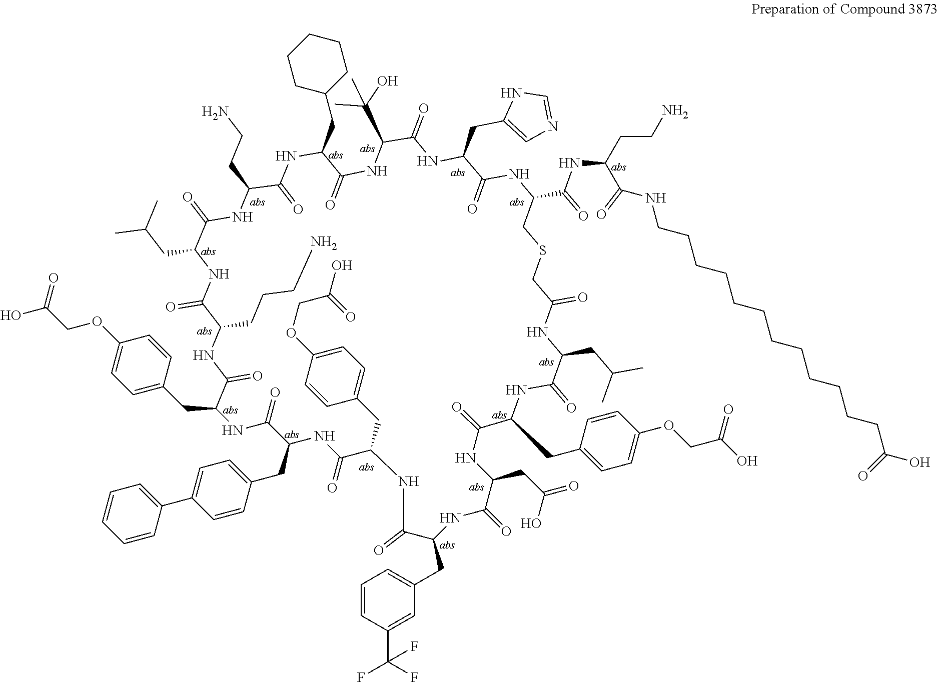 [SMILES: CC(C)C[C@@H]1NC(=O)CSC[C@@H](C(=O)N[C@@H](CCN)C(=O)NCCCCCCCCCCCCC(=O)O)NC(=O)[C@H](Cc2cnc[nH]2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3873 was prepared on a 50 μmol scale. The yield of the product was 33.2 mg, and its estimated purity by LCMS analysis was 100%. Analysis condition A: Retention time=1.74 min; ESI-MS(+) m/z [M+2H]²⁺: 1268.3.

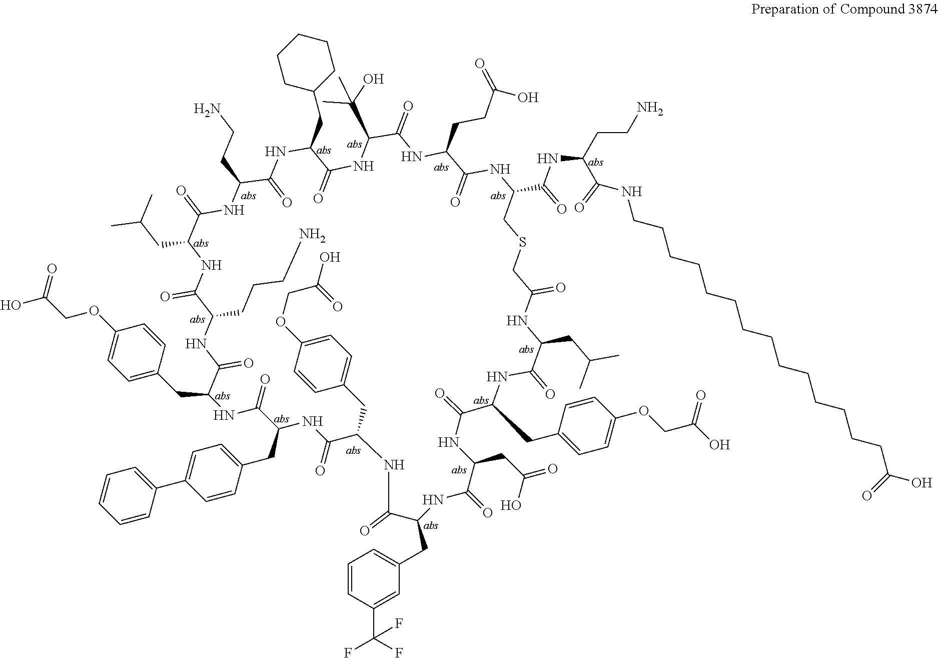 [SMILES: CC(C)C[C@@H]1NC(=O)CSC[C@@H](C(=O)N[C@@H](CCN)C(=O)NCCCCCCCCCCCCCCC(=O)O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3874 was prepared on a 50 μmol scale. The yield of the product was 20.3 mg, and its estimated purity by LCMS analysis was 96.8%. Analysis condition A: Retention time=1.82 min; ESI-MS(+) m/z [M+3H]³⁺: 852.9.

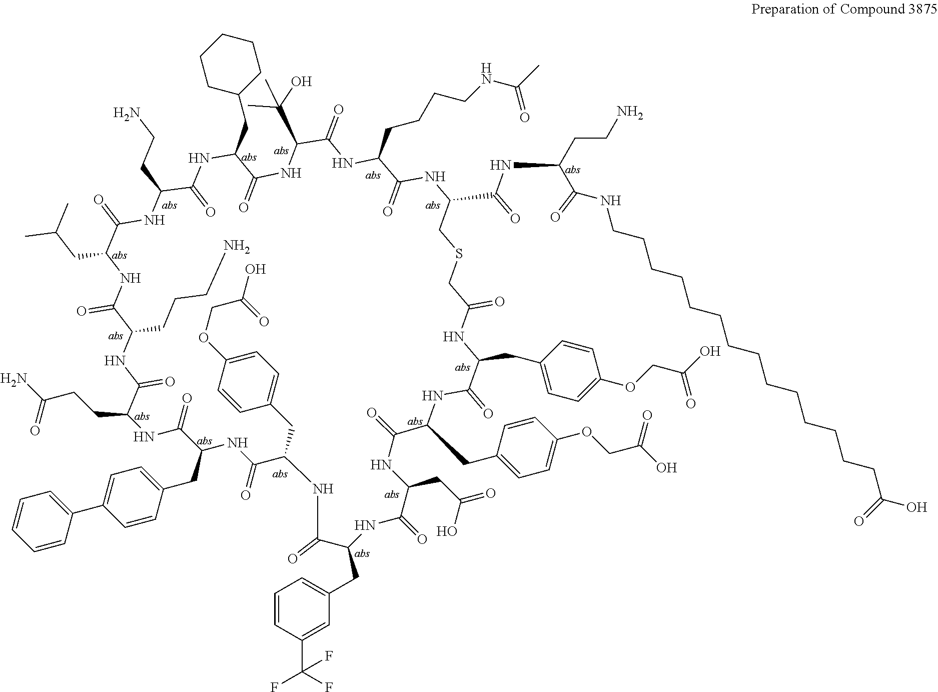 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](CCC(N)=O)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCCCC(=O)O)NC1=O]

Compound 3875 was prepared on a 50 μmol scale. The yield of the product was 36.7 mg, and its estimated purity by LCMS analysis was 97.6%. Analysis condition A: Retention time=1.7 min; ESI-MS(+) m/z [M+2H]²⁺: 1306.3.

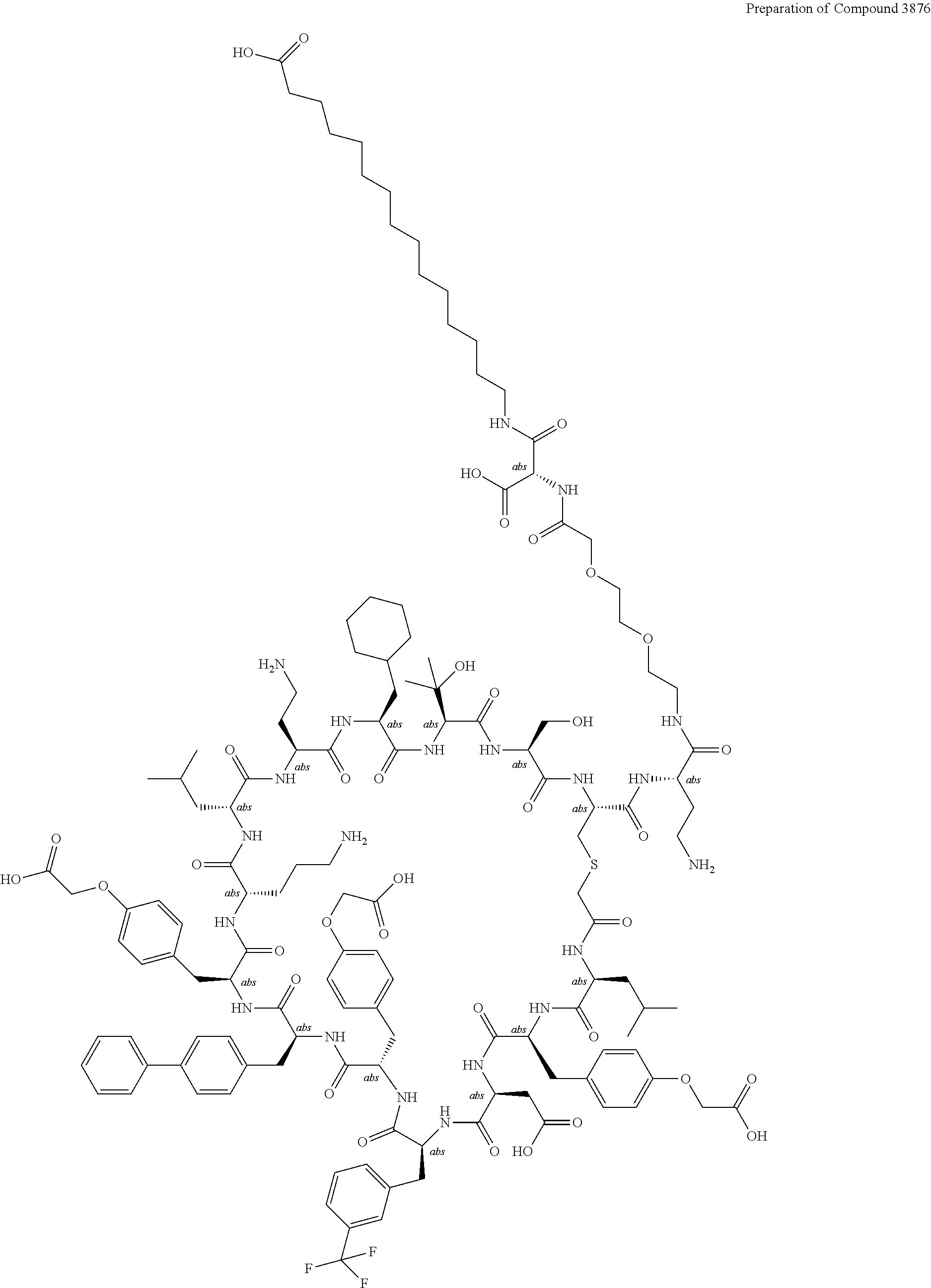 [SMILES: CC(C)C[C@@H]1NC(=O)CSC[C@@H](C(=O)N[C@@H](CCN)C(=O)NCCOCCOCC(=O)N[C@H](C(=O)O)C(=O)NCCCCCCCCCCCCCCC(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3876 was prepared on a 50 μmol scale. The yield of the product was 21.3 mg, and its estimated purity by LCMS analysis was 95%. Analysis condition A: Retention time=1.65 min; ESI-MS(+) m/z [M+2H]²⁺: 1395.

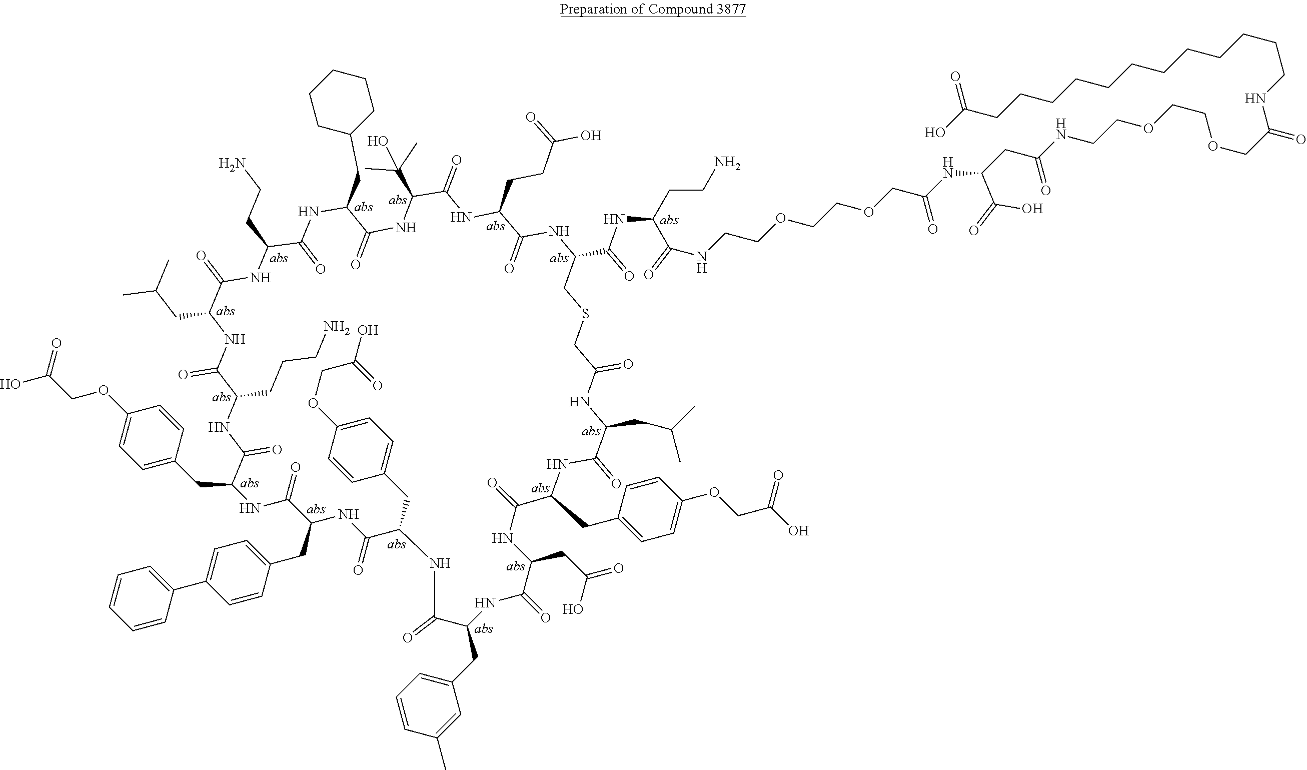 [SMILES: Cc1cccc(C[C@@H]2NC(=O)[C@H](CC(=O)O)NC(=O)[C@H](Cc3ccc(OCC(=O)O)cc3)NC(=O)[C@H](CC(C)C)NC(=O)CSC[C@@H](C(=O)N[C@@H](CCN)C(=O)NCCOCCOCC(=O)N[C@H](CC(=O)NCCOCCOCC(=O)NCCCCCCCCCCCCC(=O)O)C(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3877 was prepared on a 50 μmol scale. The yield of the product was 18.5 mg, and its estimated purity by LCMS analysis was 100%. Analysis condition A: Retention time=1.56 min; ESI-MS(+) m/z [M+2H]²⁺: 1440.3.

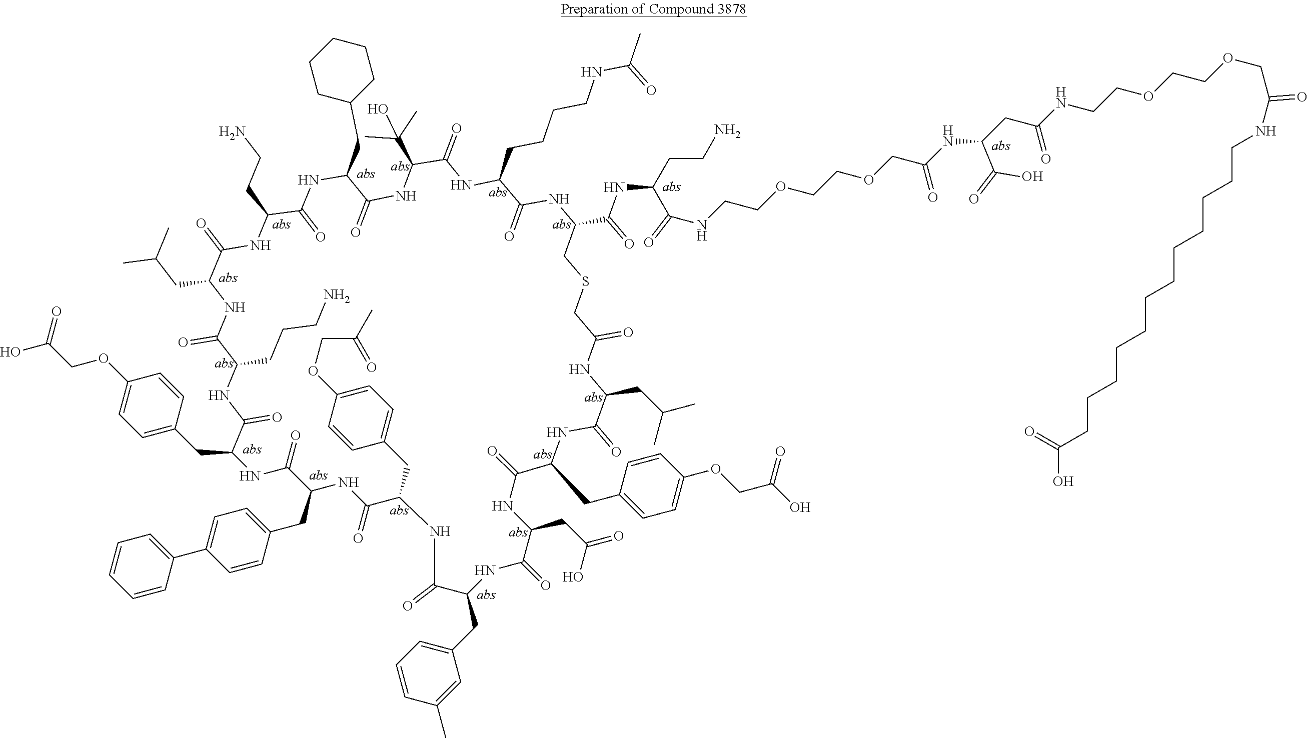 [SMILES: CC(=O)COc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CC(C)C)NC(=O)CSC[C@@H](C(=O)N[C@@H](CCN)C(=O)NCCOCCOCC(=O)N[C@H](CC(=O)NCCOCCOCC(=O)NCCCCCCCCCCCCC(=O)O)C(=O)O)NC(=O)[C@H](CCCCNC(C)=O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 3878 was prepared on a 50 μmol scale. The yield of the product was 38.7 mg, and its estimated purity by LCMS analysis was 96%. Analysis condition A: Retention time=1.69 min; ESI-MS(+) m/z [M+3H]³⁺: 974.2.

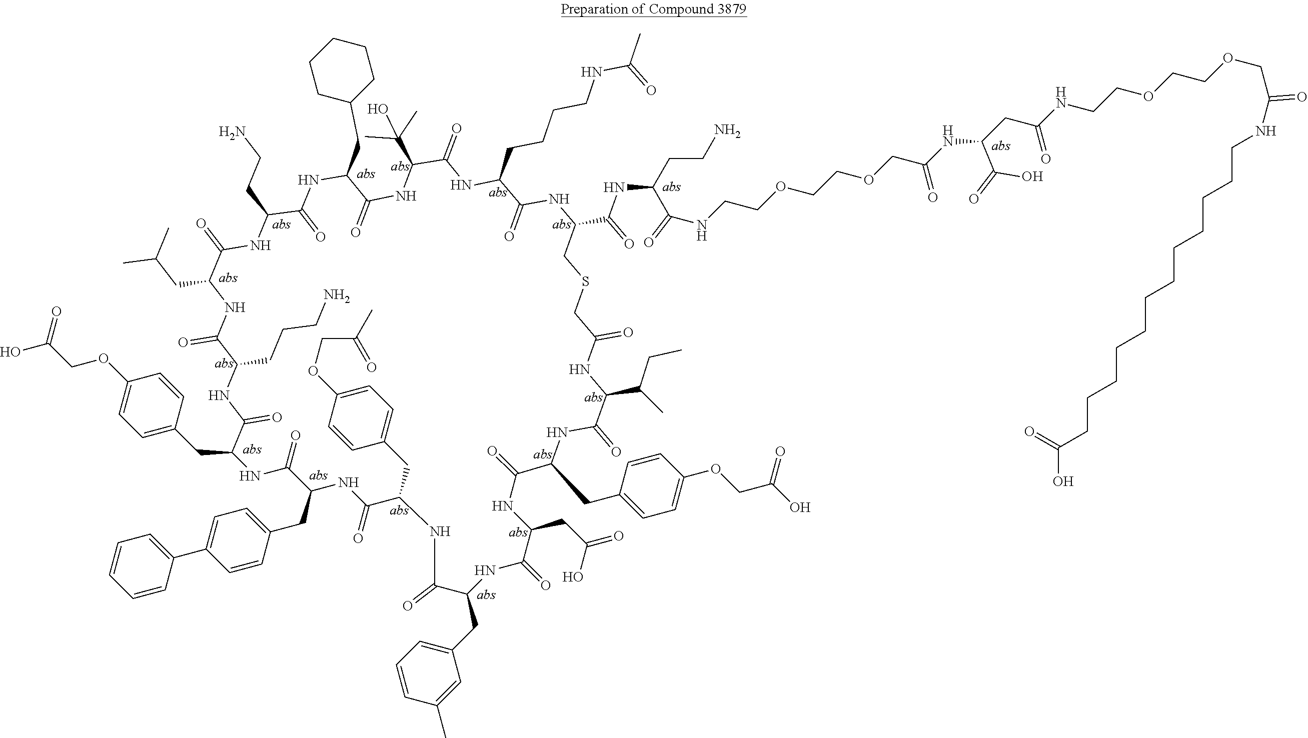 [SMILES: CCC(C)[C@@H]1NC(=O)CSC[C@@H](C(=O)N[C@@H](CCN)C(=O)NCCOCCOCC(=O)N[C@H](CC(=O)NCCOCCOCC(=O)NCCCCCCCCCCCCC(=O)O)C(=O)O)NC(=O)[C@H](CCCCNC(C)=O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(C)=O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3879 was prepared on a 50 μmol scale. The yield of the product was 35.5 mg, and its estimated purity by LCMS analysis was 95.7%. Analysis condition A: Retention time=1.72 min; ESI-MS(+) m/z [M+3H]³⁺: 974.1.

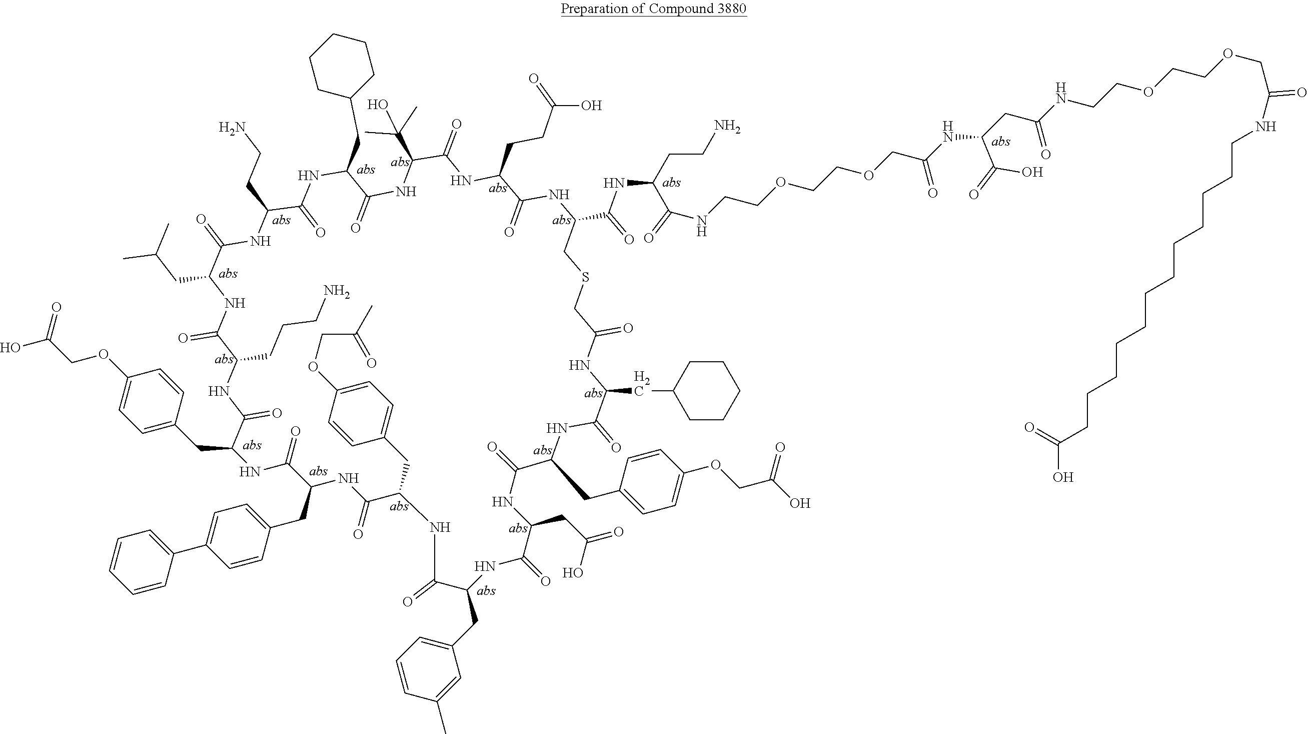 [SMILES: CC(=O)COc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CC3CCCCC3)NC(=O)CSC[C@@H](C(=O)N[C@@H](CCN)C(=O)NCCOCCOCC(=O)N[C@H](CC(=O)NCCOCCOCC(=O)NCCCCCCCCCCCCC(=O)O)C(=O)O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 3880 was prepared on a 50 μmol scale. The yield of the product was 11.5 mg, and its estimated purity by LCMS analysis was 97.1%. Analysis condition B: Retention time=1.61 min; ESI-MS(+) m/z [M+2H]²⁺: 1461.

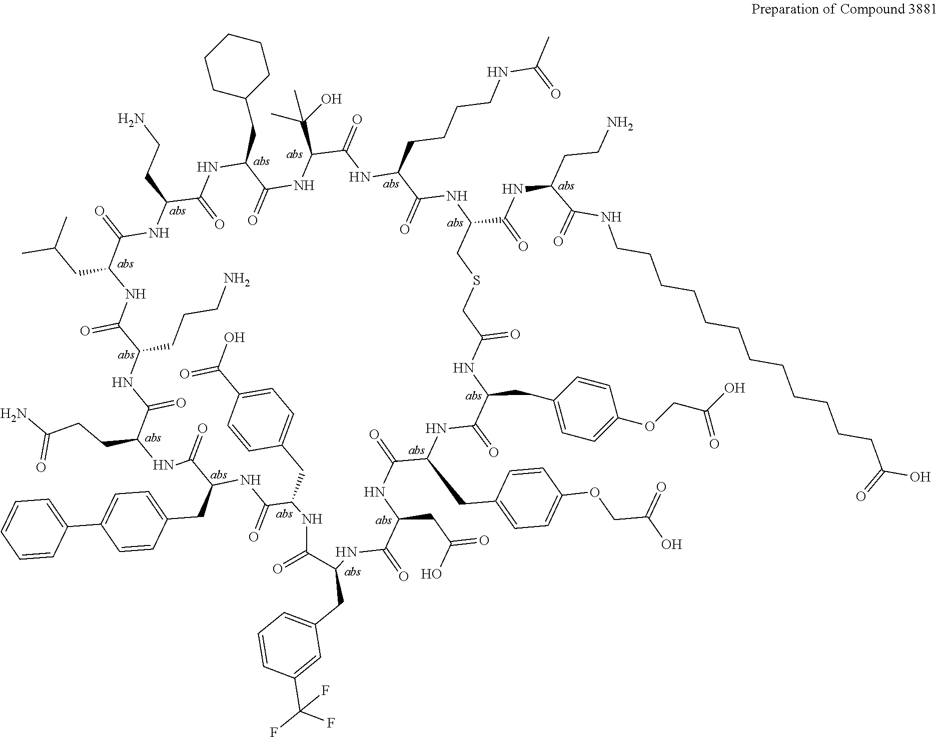 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](CCC(N)=O)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCCCCCCCCCCCC(=O)O)NC1=O]

Compound 3881 was prepared on a 50 μmol scale. The yield of the product was 39.8 mg, and its estimated purity by LCMS analysis was 95.4%. Analysis condition A: Retention time=1.44 min; ESI-MS(+) m/z [M+3H]³⁺: 852.2.

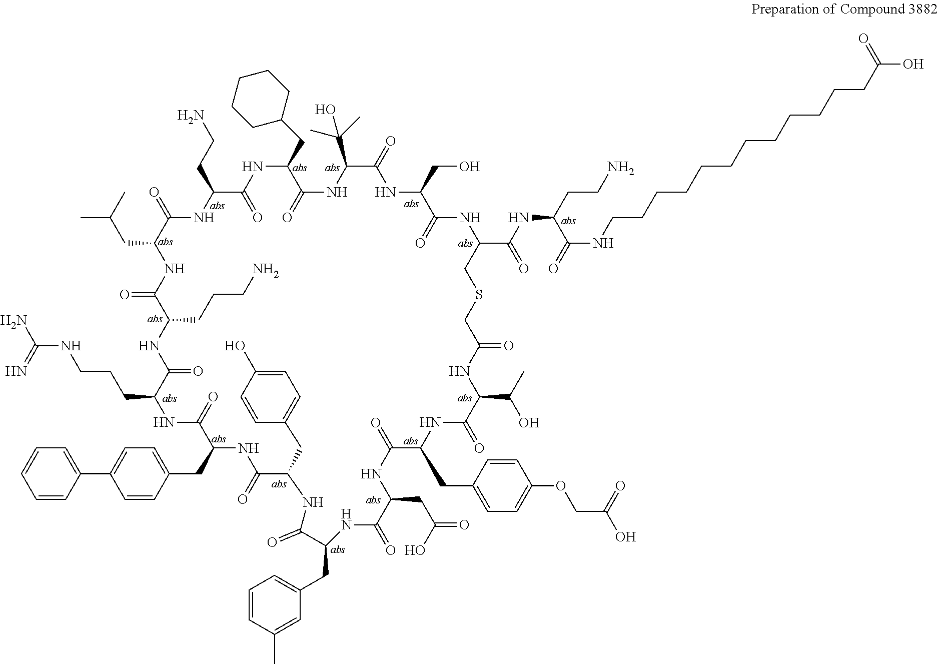 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSCC(C(=O)N[C@@H](CCN)C(=O)NCCCCCCCCCCCCC(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=N)N)NC(=O)[C@H](Cc3ccc(-c4ccccc4)cc3)NC(=O)[C@H](Cc3ccc(O)cc3)NC2=O)c1]

Compound 3882 was prepared on a 50 μmol scale. The yield of the product was 10.8 mg, and its estimated purity by LCMS analysis was 96%. Analysis condition A: Retention time=1.54 min; ESI-MS(+) m/z [M+2H]²⁺: 1149.

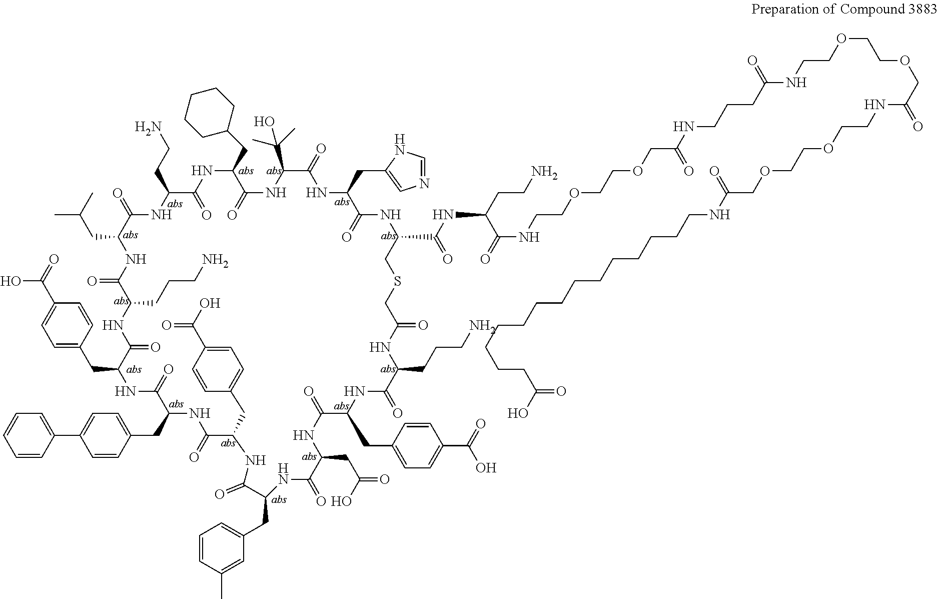 [SMILES: Cc1cccc(C[C@@H]2NC(=O)[C@H](CC(=O)O)NC(=O)[C@H](Cc3ccc(C(=O)O)cc3)NC(=O)[C@H](CCCN)NC(=O)CSC[C@@H](C(=O)N[C@@H](CCN)C(=O)NCCOCCOCC(=O)NCCCC(=O)NCCOCCOCC(=O)NCCOCCOCC(=O)NCCCCCCCCCCCCCCC(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3883 was prepared on a 50 μmol scale. The yield of the product was 7.1 mg, and its estimated purity by LCMS analysis was 100%. Analysis condition A: Retention time=1.68 min; ESI-MS(+) m/z [M+2H]²⁺: 1501.1.

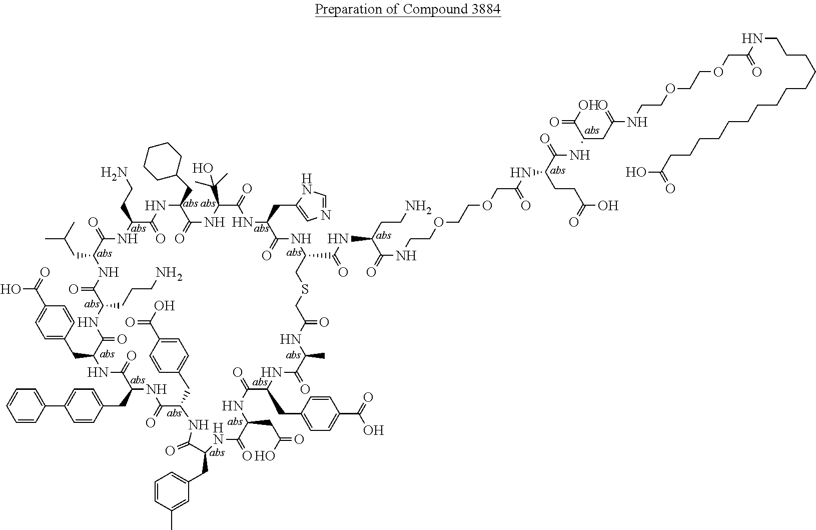 [SMILES: Cc1cccc(C[C@@H]2NC(=O)[C@H](CC(=O)O)NC(=O)[C@H](Cc3ccc(C(=O)O)cc3)NC(=O)[C@H](C)NC(=O)CSC[C@@H](C(=O)N[C@@H](CCN)C(=O)NCCOCCOCC(=O)N[C@@H](CCC(=O)O)C(=O)N[C@@H](CC(=O)NCCOCCOCC(=O)NCCCCCCCCCCCCCCC(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3884 was prepared on a 50 μmol scale. The yield of the product was 5.7 mg, and its estimated purity by LCMS analysis was 97%. Analysis condition B: Retention time=1.83 min; ESI-MS(+) m/z [M+2H]²⁺: 1457.

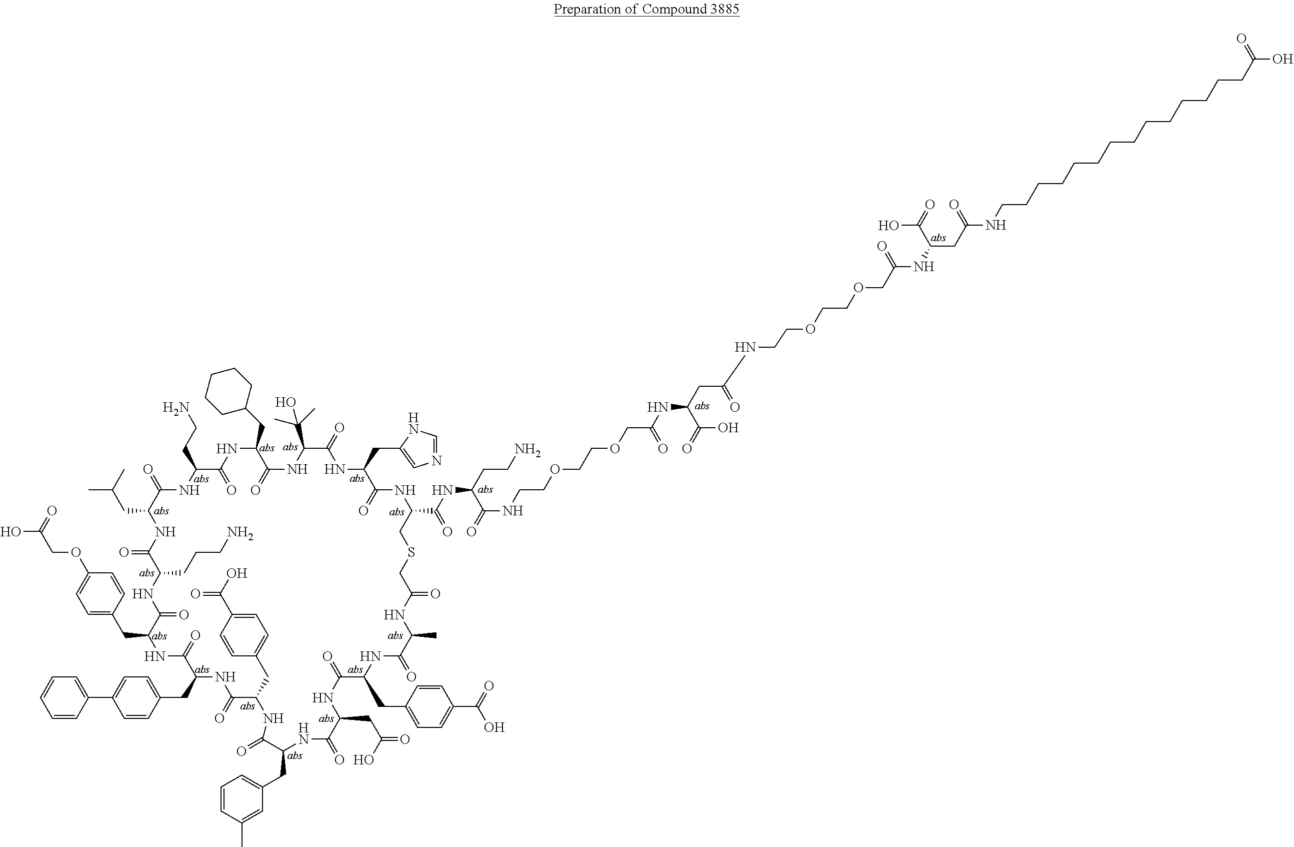 [SMILES: Cc1cccc(C[C@@H]2NC(=O)[C@H](CC(=O)O)NC(=O)[C@H](Cc3ccc(C(=O)O)cc3)NC(=O)[C@H](C)NC(=O)CSC[C@@H](C(=O)N[C@@H](CCN)C(=O)NCCOCCOCC(=O)N[C@@H](CC(=O)NCCOCCOCC(=O)N[C@@H](CC(=O)NCCCCCCCCCCCCCCC(=O)O)C(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 3885 was prepared on a 50 μmol scale. The yield of the product was 5.6 mg, and its estimated purity by LCMS analysis was 98.8%. Analysis condition B: Retention time=1.82 min; ESI-MS(+) m/z [M+2H]²⁺: 1450.1.

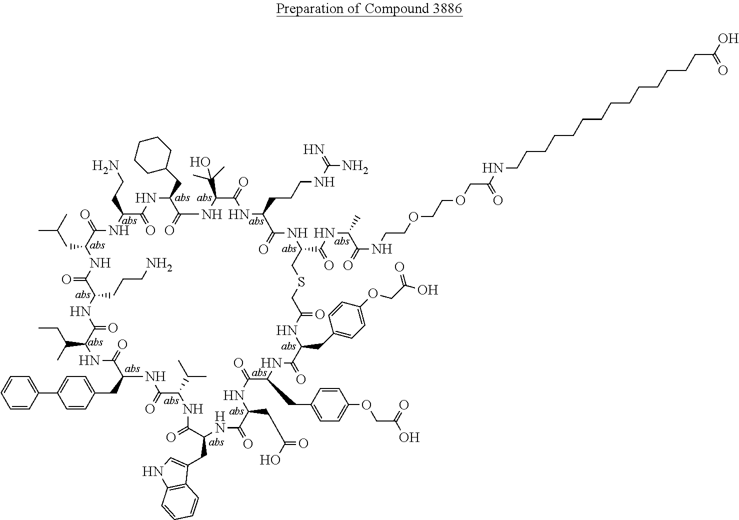 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@H](C)C(=O)NCCOCCOCC(=O)NCCCCCCCCCCCCCCC(=O)O)NC(=O)[C@H](CCCNC(=N)N)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC1=O]

Compound 3886 was prepared on a 50 μmol scale. The yield of the product was 3.2 mg, and its estimated purity by LCMS analysis was 100%. Analysis condition A: Retention time-1.78 min; ESI-MS(+) m/z [M+3H]³⁺: 850.

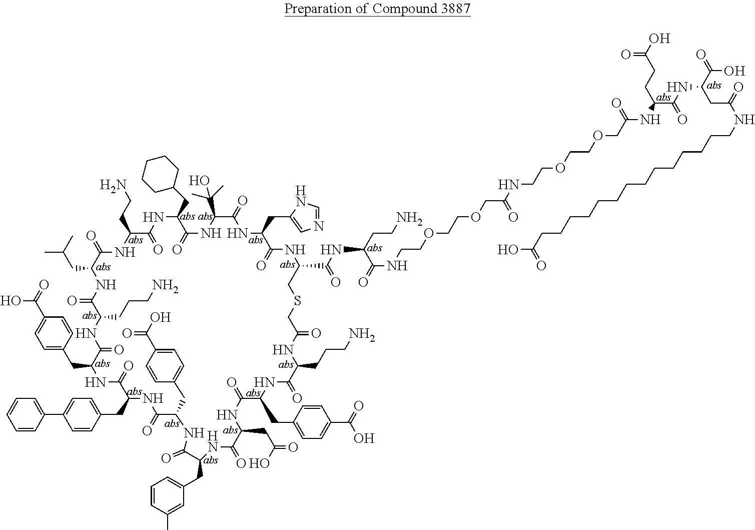 [SMILES: Cc1cccc(C[C@@H]2NC(=O)[C@H](CC(=O)O)NC(=O)[C@H](Cc3ccc(C(=O)O)cc3)NC(=O)[C@H](CCCN)NC(=O)CSC[C@@H](C(=O)N[C@@H](CCN)C(=O)NCCOCCOCC(=O)NCCOCCOCC(=O)N[C@@H](CCC(=O)O)C(=O)N[C@@H](CC(=O)NCCCCCCCCCCCCCCC(=O)O)C(=O)O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3887 was prepared on a 50 μmol scale. The yield of the product was 9.3 mg, and its estimated purity by LCMS analysis was 97.7%. Analysis condition B: Retention time=1.72 min; ESI-MS(+) m/z [M+2H]²⁺: 1478.2.

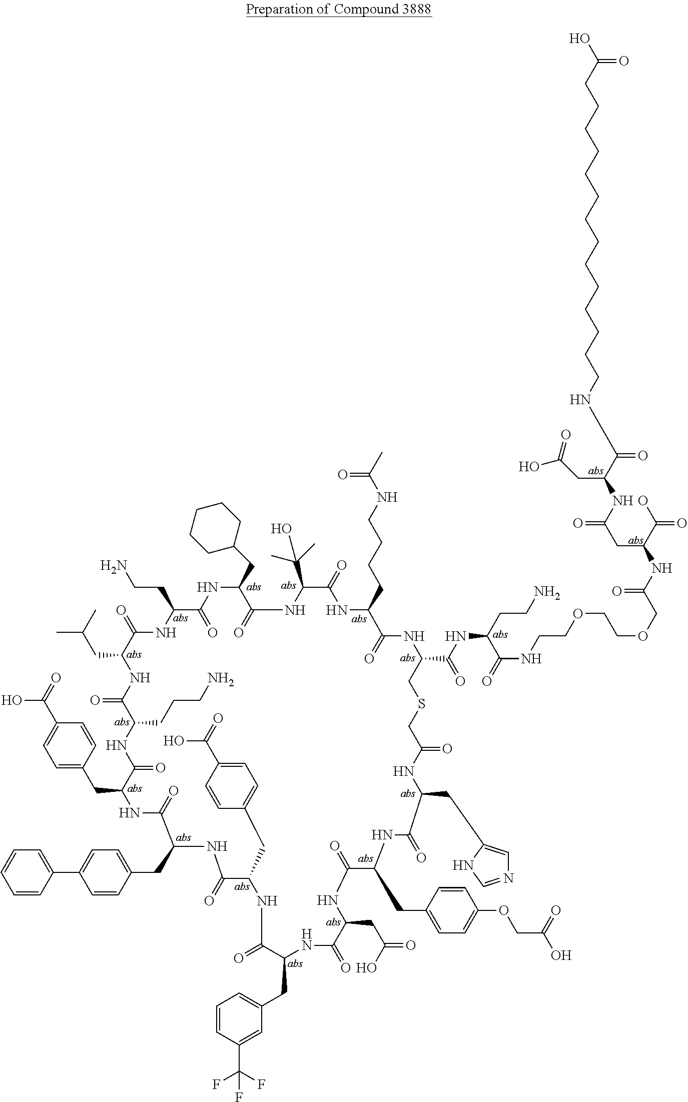 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCOCCOCC(=O)N[C@@H](CC(=O)N[C@@H](CC(=O)O)C(=O)NCCCCCCCCCCCCCCC(=O)O)C(=O)O)NC1=O]

Compound 3888 was prepared on a 50 μmol scale. The yield of the product was 10 mg, and its estimated purity by LCMS analysis was 95.3%. Analysis condition A: Retention time=1.54 min; ESI-MS(+) m/z [M+2H]²⁺: 1468.1.

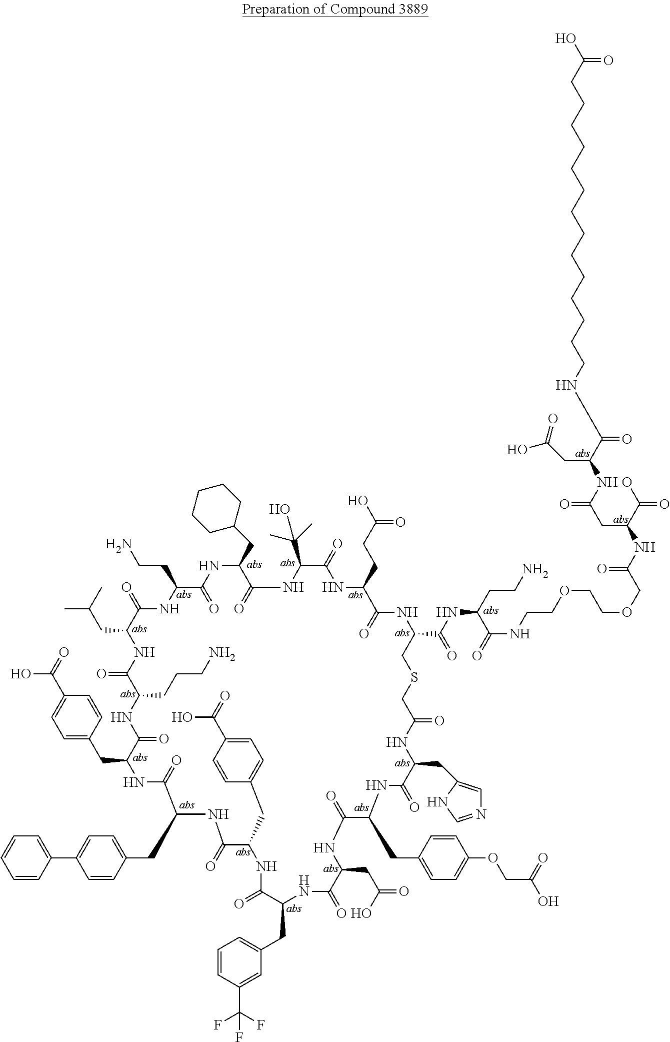 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CCN)C(=O)NCCOCCOCC(=O)N[C@@H](CC(=O)N[C@@H](CC(=O)O)C(=O)NCCCCCCCCCCCCCCC(=O)O)C(=O)O)NC(=O)[C@H](CCC(=O)O)NC(=O)[C@H](C(C)(C)O)NC(=O)[C@H](CC2CCCCC2)NC(=O)[C@H](CCN)NC1=O]

Compound 3889 was prepared on a 50 μmol scale. The yield of the product was 11.9 mg, and its estimated purity by LCMS analysis was 100%. Analysis condition A: Retention time=1.66 min; ESI-MS(+) m/z [M+2H]²⁺: 1448.2.

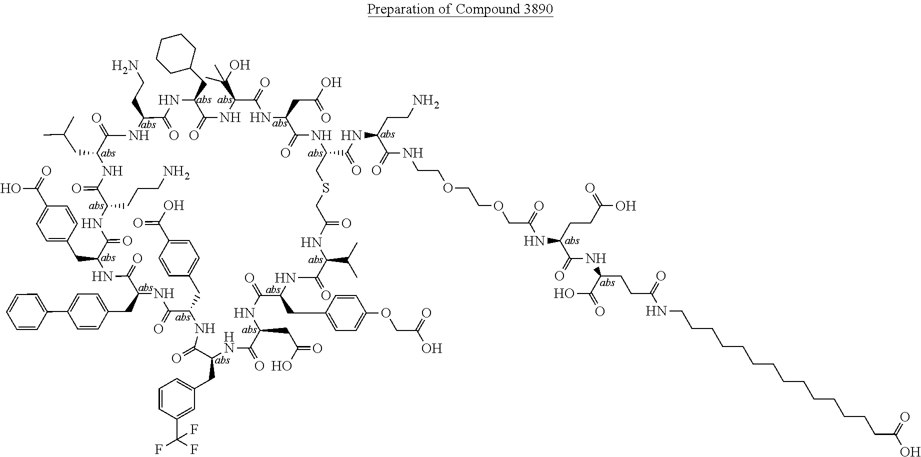 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)C)NC(=O)CSC[C@@H](C(=O)N[C@@H](CCN)C(=O)NCCOCCOCC(=O)N[C@@H](CCC(=O)O)C(=O)N[C@@H](CCC(=O)NCCCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC1=O]

Compound 3890 was prepared on a 50 μmol scale. The yield of the product was 10.2 mg, and its estimated purity by LCMS analysis was 95.7%. Analysis condition B: Retention time=1.93 min; ESI-MS(+) m/z [M+2H]²⁺: 1436.1.

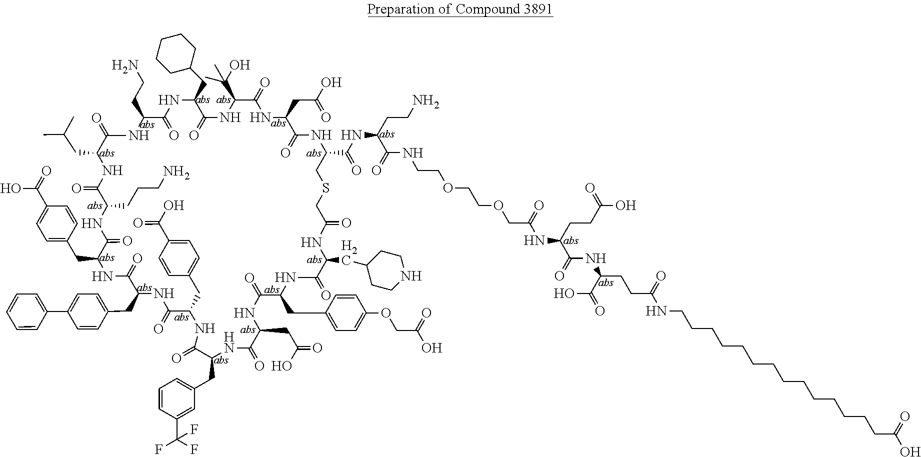 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CC2CCNCC2)NC(=O)CSC[C@@H](C(=O)N[C@@H](CCN)C(=O)NCCOCCOCC(=O)N[C@@H](CCC(=O)O)C(=O)N[C@@H](CCC(=O)NCCCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC1=O]

Compound 3891 was prepared on a 50 μmol scale. The yield of the product was 6.2 mg, and its estimated purity by LCMS analysis was 95.4%. Analysis condition A: Retention time=1.63 min; ESI-MS(+) m/z [M+2H]²⁺: 1450.

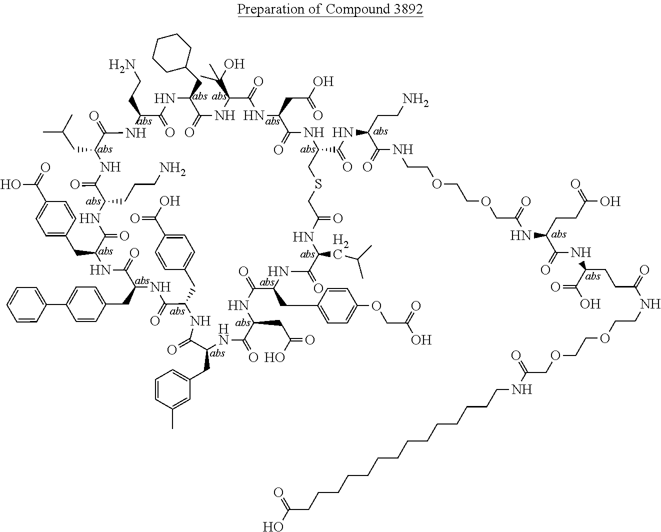 [SMILES: Cc1cccc(C[C@@H]2NC(=O)[C@H](CC(=O)O)NC(=O)[C@H](Cc3ccc(OCC(=O)O)cc3)NC(=O)[C@H](CC(C)C)NC(=O)CSC[C@@H](C(=O)N[C@@H](CCN)C(=O)NCCOCCOCC(=O)N[C@@H](CCC(=O)O)C(=O)N[C@@H](CCC(=O)NCCOCCOCC(=O)NCCCCCCCCCCCCCCC(=O)O)C(=O)O)NC(=O)[C@H](CC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3892 was prepared on a 50 μmol scale. The yield of the product was 14.1 mg, and its estimated purity by LCMS analysis was 94.6%. Analysis condition A: Retention time=1.5 min; ESI-MS(+) m/z [M+2H]²⁺: 1489.1.

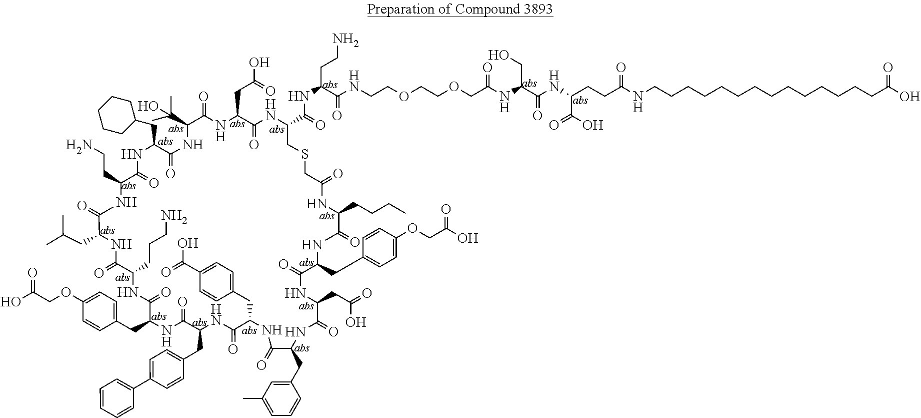 [SMILES: CCCC[C@@H]1NC(=O)CSC[C@@H](C(=O)N[C@@H](CCN)C(=O)NCCOCCOCC(=O)N[C@@H](CO)C(=O)N[C@H](CCC(=O)NCCCCCCCCCCCCCCC(=O)O)C(=O)O)NC(=O)[C@H](CC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3893 was prepared on a 50 μmol scale. The yield of the product was 7 mg, and its estimated purity by LCMS analysis was 99.3%. Analysis condition B: Retention time=1.93 min; ESI-MS(+) m/z [M+2H]²⁺: 1409.9.

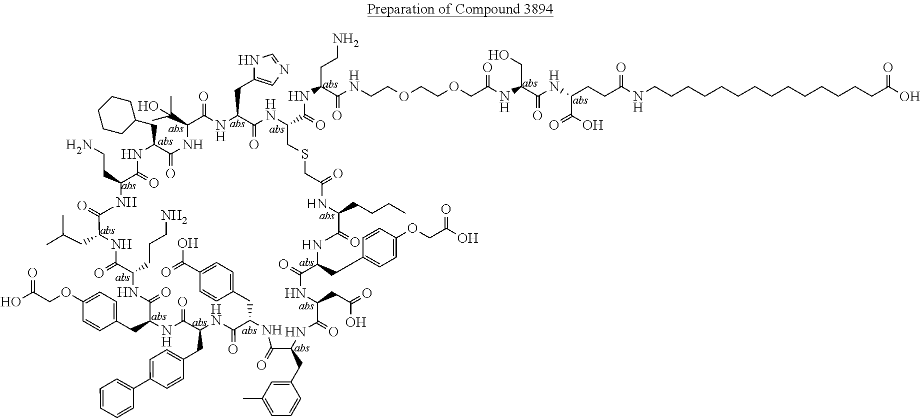 [SMILES: CCCC[C@@H]1NC(=O)CSC[C@@H](C(=O)N[C@@H](CCN)C(=O)NCCOCCOCC(=O)N[C@@H](CO)C(=O)N[C@H](CCC(=O)NCCCCCCCCCCCCCCC(=O)O)C(=O)O)NC(=O)[C@H](Cc2cnc[nH]2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 3894 was prepared on a 50 μmol scale. The yield of the product was 2.2 mg, and its estimated purity by LCMS analysis was 96.3%. Analysis condition A: Retention time=1.62 min; ESI-MS(+) m/z [M+2H]²⁺: 1421.

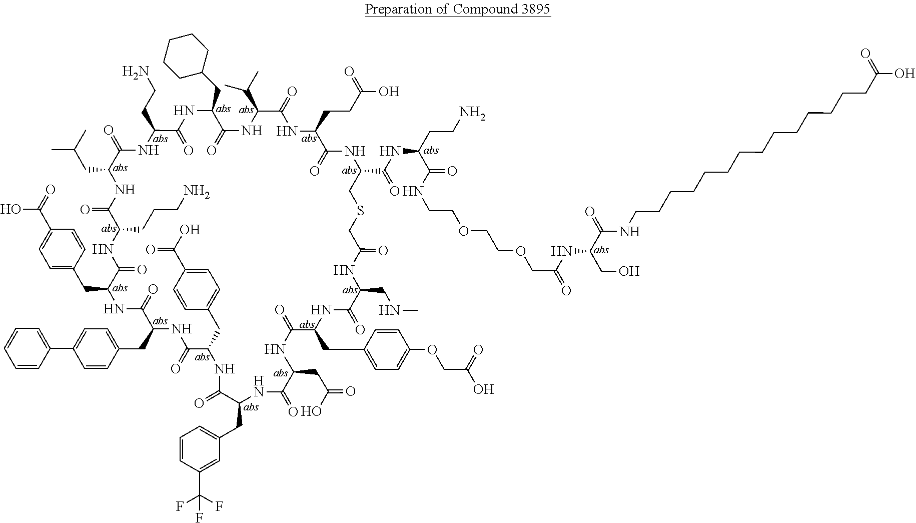 [SMILES: CNC[C@@H]1NC(=O)CSC[C@@H](C(=O)N[C@@H](CCN)C(=O)NCCOCCOCC(=O)N[C@@H](CO)C(=O)NCCCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3895 was prepared on a 50 μmol scale. The yield of the product was 2.7 mg, and its estimated purity by LCMS analysis was 96.6%. Analysis condition B: Retention time=1.86 min; ESI-MS(+) m/z [M+2H]²⁺: 1350.4.

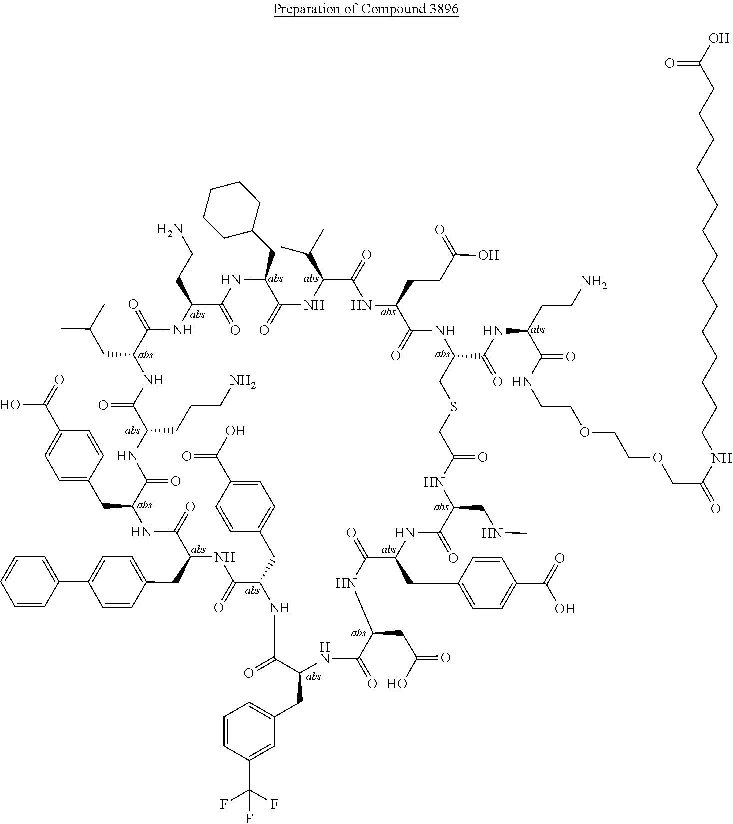 [SMILES: CNC[C@@H]1NC(=O)CSC[C@@H](C(=O)N[C@@H](CCN)C(=O)NCCOCCOCC(=O)NCCCCCCCCCCCCCCC(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC1=O]

Compound 3896 was prepared on a 50 μmol scale. The yield of the product was 4.1 mg, and its estimated purity by LCMS analysis was 96.4%. Analysis condition A: Retention time=1.68 min; ESI-MS(+) m/z [M+2H]²⁺: 861.5.

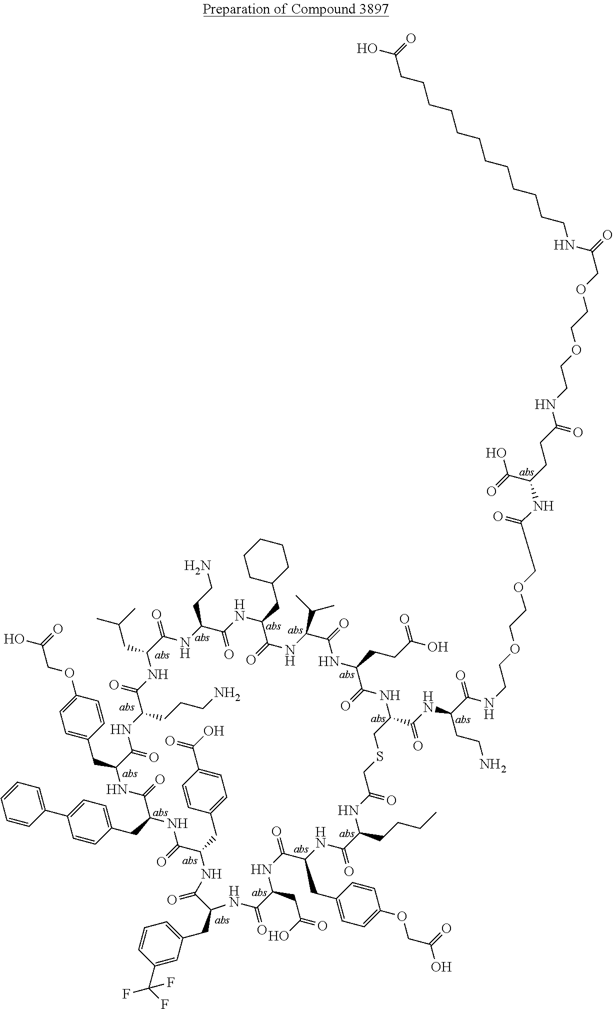 [SMILES: CCCC[C@@H]1NC(=O)CSC[C@@H](C(=O)N[C@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3897 was prepared on a 50 μmol scale. The yield of the product was 7.3 mg, and its estimated purity by LCMS analysis was 100%. Analysis condition A: Retention time=1.66 min; ESI-MS(+) m/z [M+2H]²⁺: 1451.1.

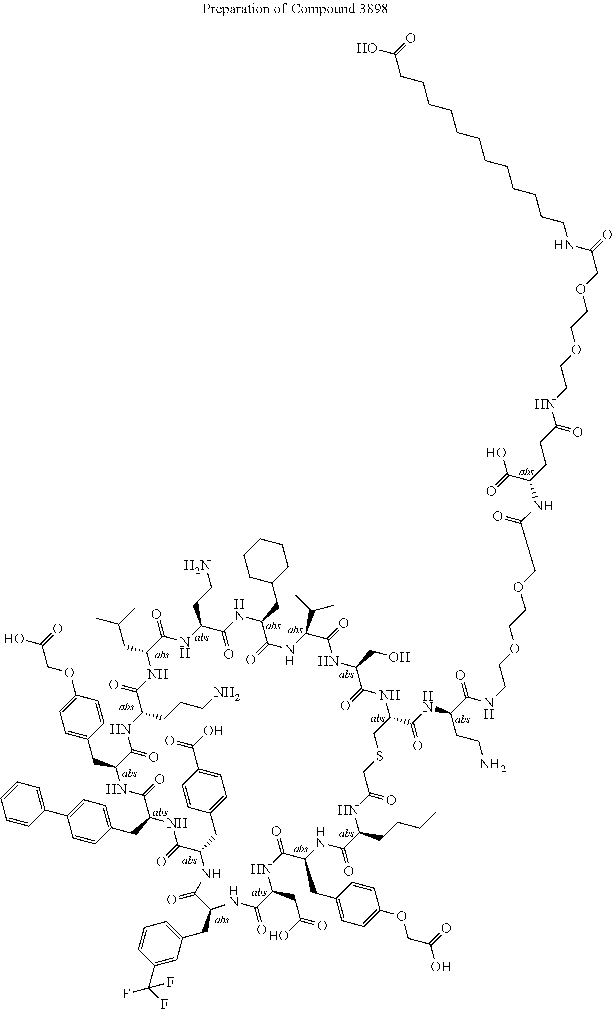 [SMILES: CCCC[C@@H]1NC(=O)CSC[C@@H](C(=O)N[C@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3898 was prepared on a 50 μmol scale. The yield of the product was 8.9 mg, and its estimated purity by LCMS analysis was 95.2%. Analysis condition B: Retention time=1.96 min; ESI-MS(+) m/z [M+2H]²⁺: 959.2.

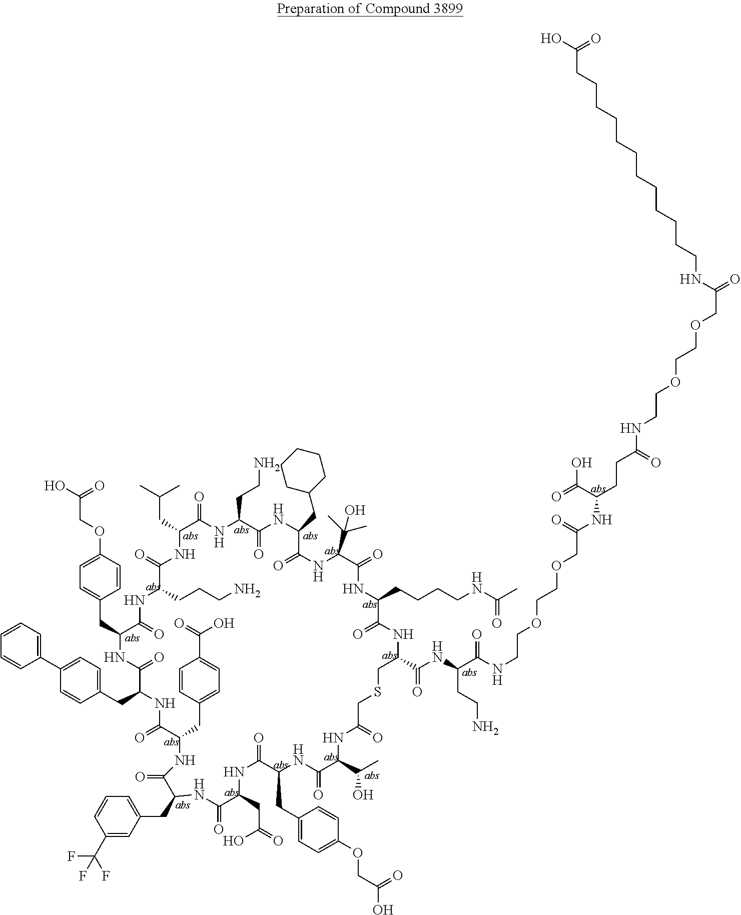 [SMILES: CC(=O)NCCCC[C@@H]1NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H]([C@H](C)O)NC(=O)CSC[C@@H](C(=O)N[C@H](CCN)C(=O)NCCOCCOCC(=O)N[C@@H](CCC(=O)NCCOCCOCC(=O)NCCCCCCCCCCCCC(=O)O)C(=O)O)NC1=O]

Compound 3899 was prepared on a 50 μmol scale. The yield of the product was 6 mg, and its estimated purity by LCMS analysis was 96.3%. Analysis condition A: Retention time=1.65 min; ESI-MS(+) m/z [M+2H]²⁺: 1473.9.

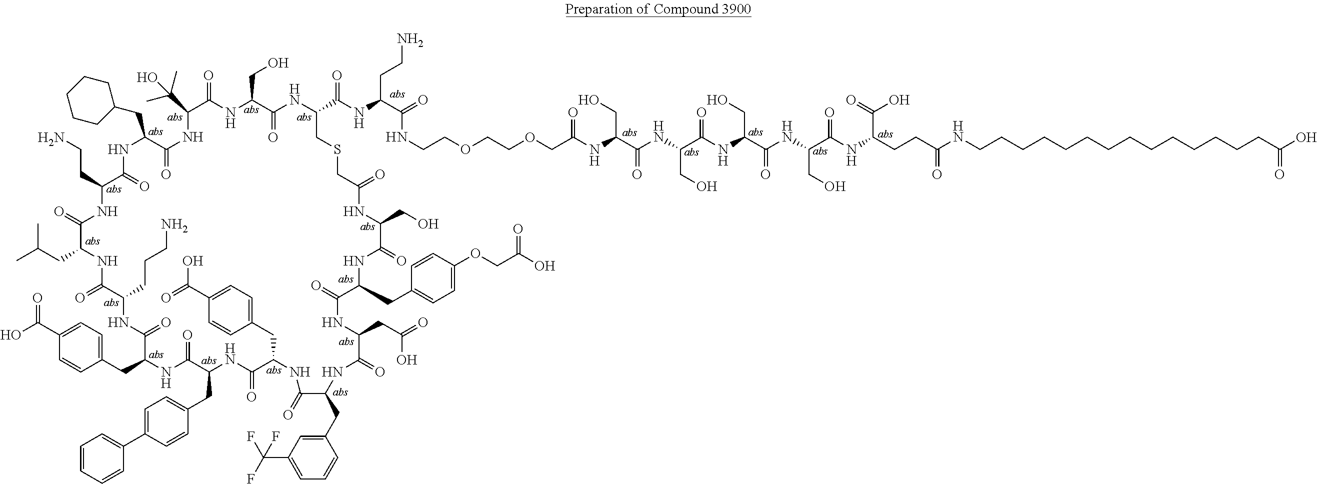 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CO)NC(=O)CSC[C@@H](C(=O)N[C@@H](CCN)C(=O)NCCOCCOCC(=O)N[C@@H](CO)C(=O)N[C@@H](CO)C(=O)N[C@@H](CO)C(=O)N[C@@H](CO)C(=O)N[C@@H](CCC(=O)NCCCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Compound 3900 was prepared on a 50 μmol scale. The yield of the product was 2.5 mg, and its estimated purity by LCMS analysis was 98.8%. Analysis condition B: Retention time=1.92 min; ESI-MS(+) m/z [M+2H]²⁺: 1017.1.

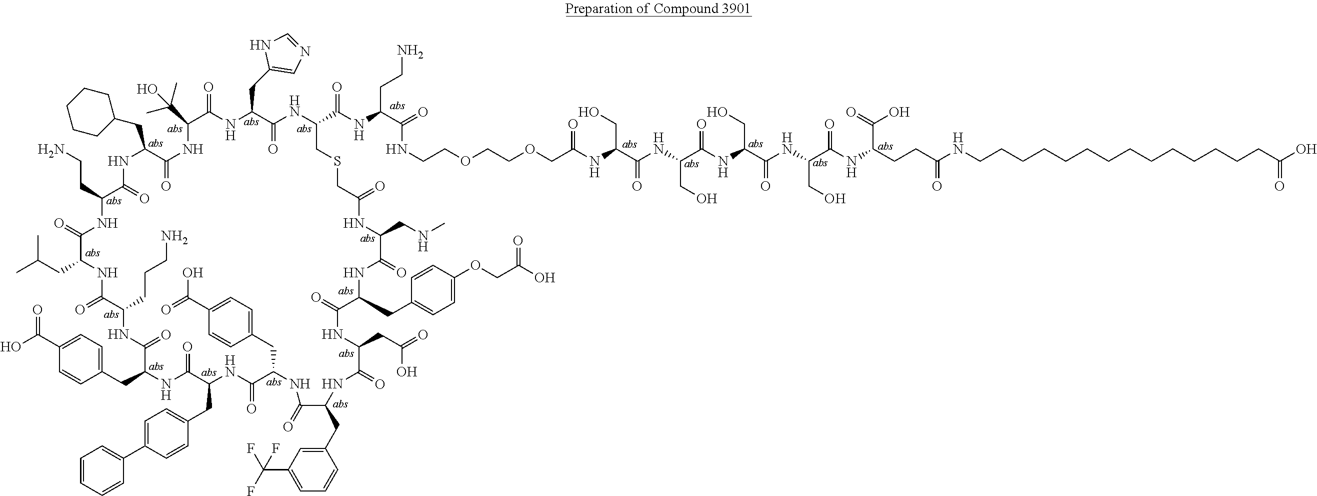 [SMILES: CNC[C@@H]1NC(=O)CSC[C@@H](C(=O)N[C@@H](CCN)C(=O)NCCOCCOCC(=O)N[C@@H](CO)C(=O)N[C@@H](CO)C(=O)N[C@@H](CO)C(=O)N[C@@H](CO)C(=O)N[C@@H](CCC(=O)NCCCCCCCCCCCCCCC(=O)O)C(=O)O)NC(=O)[C@H](Cc2cnc[nH]2)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3901 was prepared on a 50 μmol scale. The yield of the product was 4.8 mg, and its estimated purity by LCMS analysis was 95.3%. Analysis condition B: Retention time=1.82 min; ESI-MS(+) m/z [M+2H]²⁺: 1558.1.

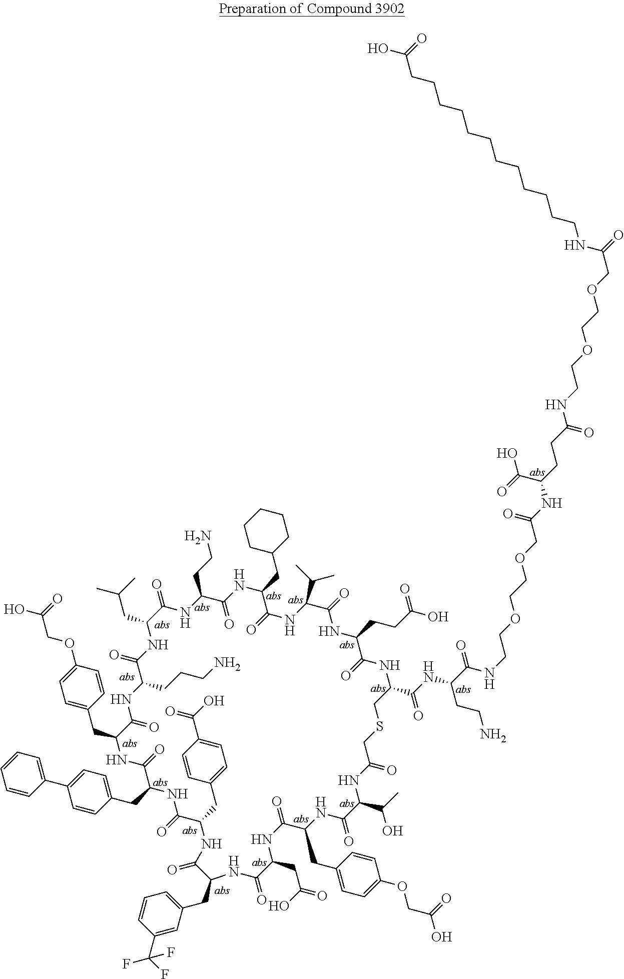 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CCC(=O)O)NC(=O)[C@H](C(C)C)NC(=O)[C@H](CC2CCCCC2)NC(=O)[C@H](CCN)NC1=O]

Compound 3902 was prepared on a 50 μmol scale. The yield of the product was 2.5 mg, and its estimated purity by LCMS analysis was 96.3%. Analysis condition B: Retention time=1.9 min; ESI-MS(+) m/z [M+2H]²⁺: 1445.2.

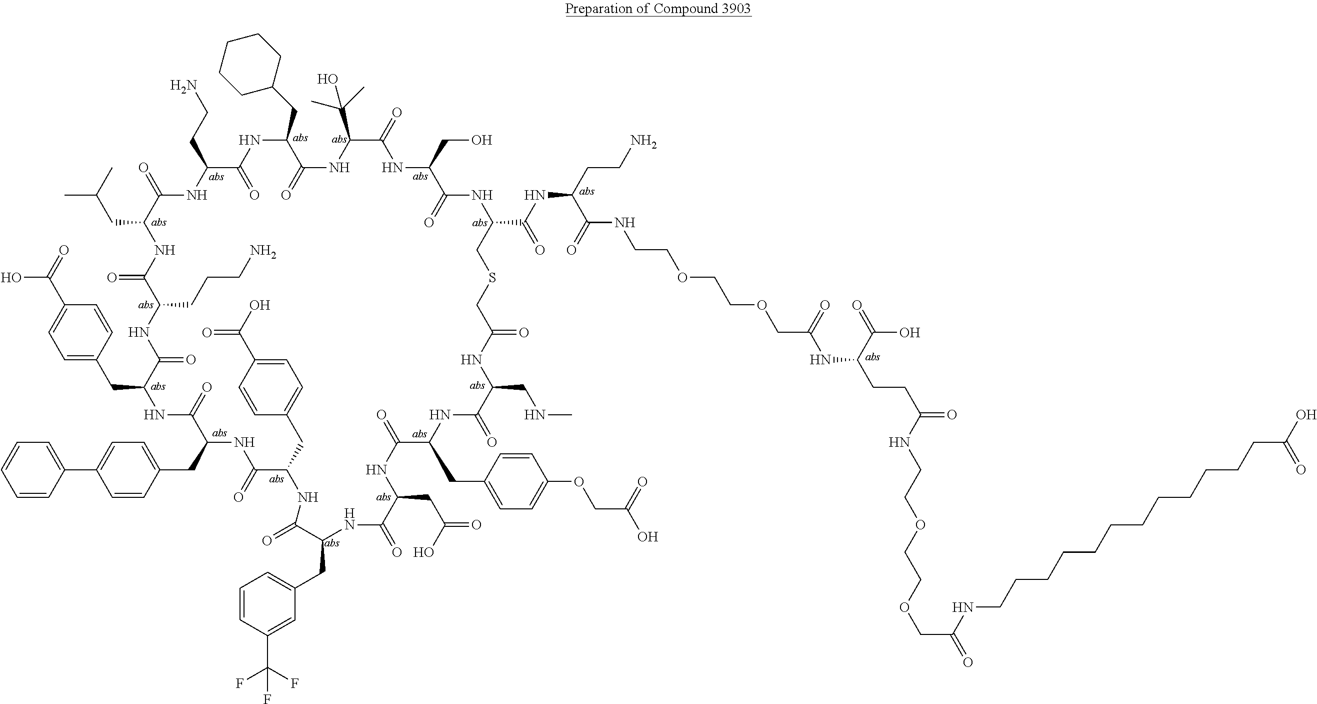 [SMILES: CNC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3903 was prepared on a 50 μmol scale. The yield of the product was 7 mg, and its estimated purity by LCMS analysis was 95.4%. Analysis condition B: Retention time=1.75 min; ESI-MS(+) m/z [M+3H]³⁺: 945.

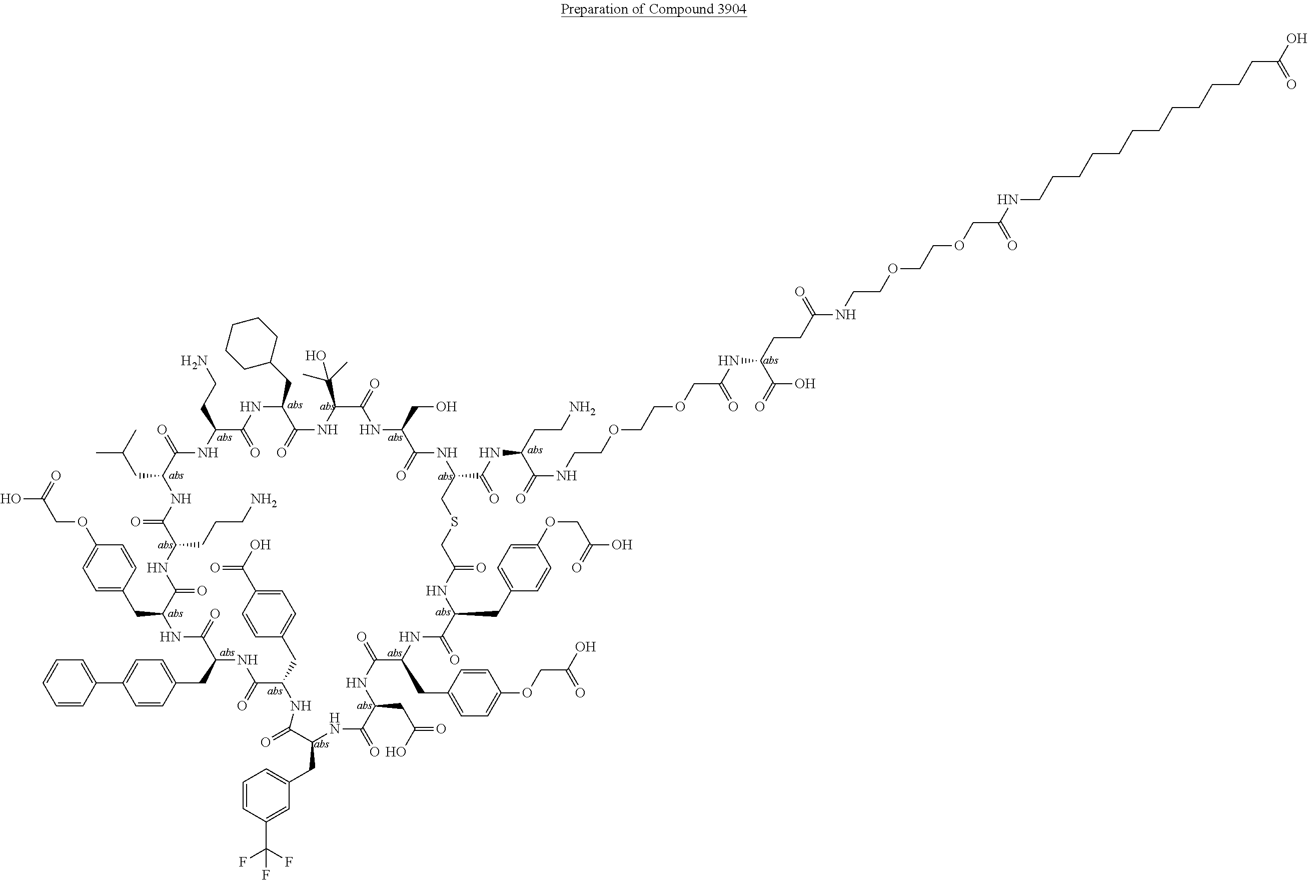 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CCN)C(=O)NCCOCCOCC(=O)N[C@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC1=O]

Compound 3904 was prepared on a 50 μmol scale. The yield of the product was 21 mg, and its estimated purity by LCMS analysis was 100%. Analysis condition B: Retention time=1.92 min; ESI-MS(+) m/z [M+2H]²⁺: 1492.2.

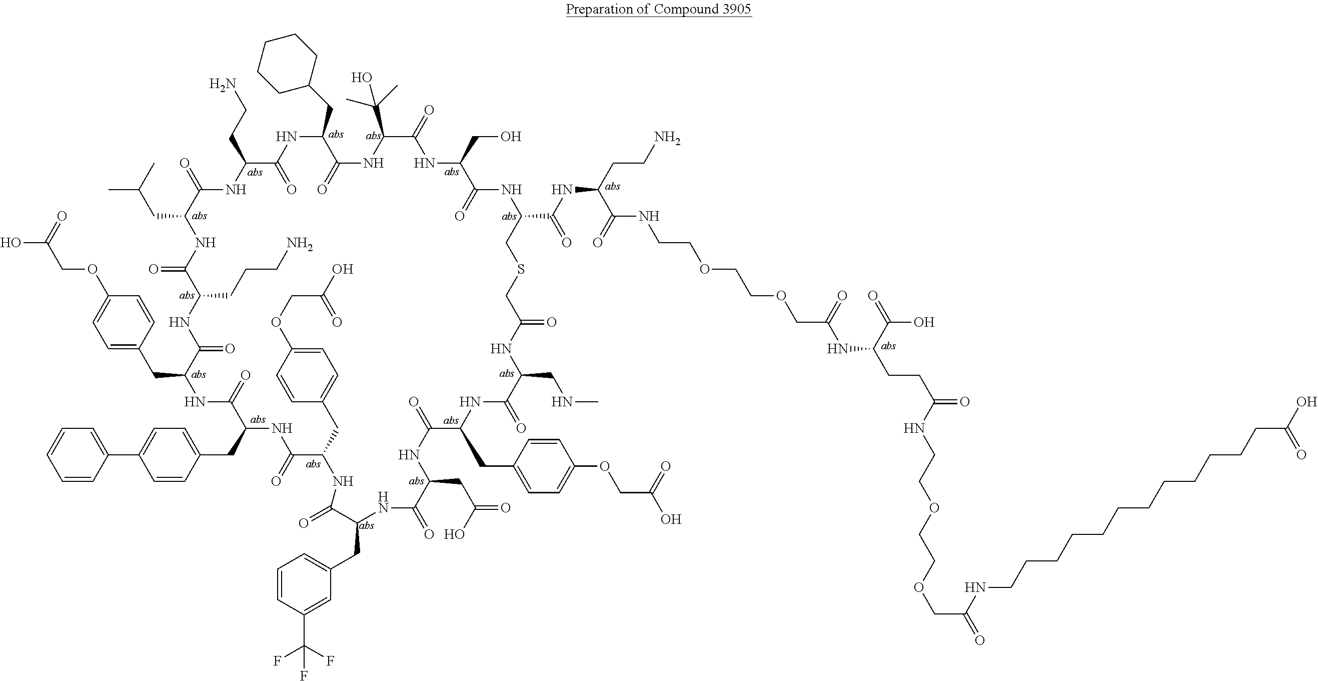 [SMILES: CNC[C@@H]1NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Compound 3905 was prepared on a 50 μmol scale. The yield of the product was 5.9 mg, and its estimated purity by LCMS analysis was 96.7%. Analysis condition B: Retention time=1.77 min; ESI-MS(+) m/z [M+3H]³⁺: 965.2.

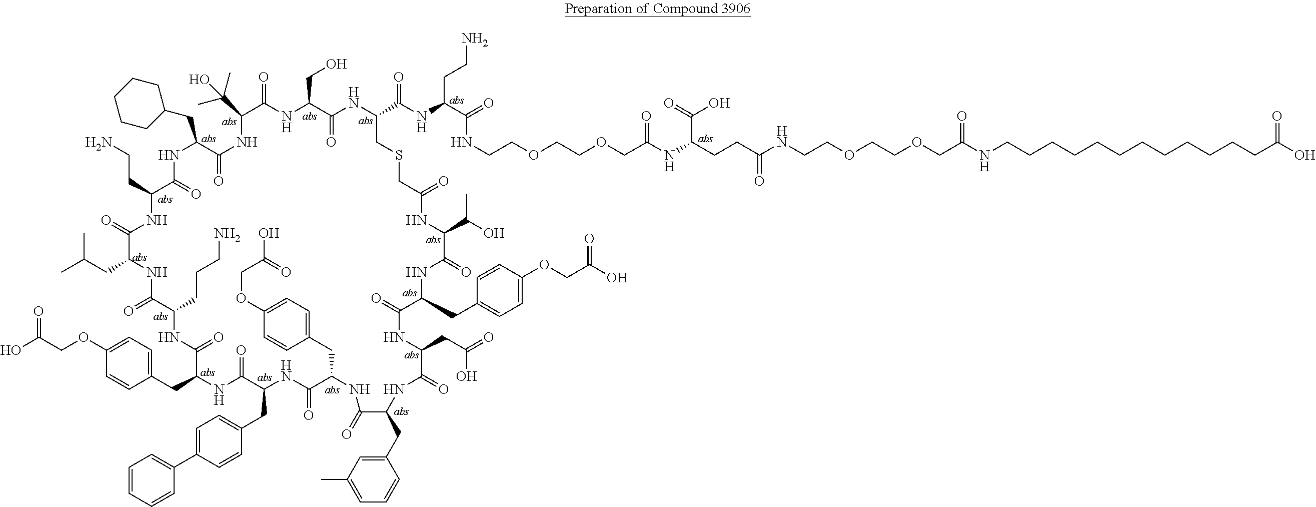 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 3906 was prepared on a 50 μmol scale. The yield of the product was 7.3 mg, and its estimated purity by LCMS analysis was 97.9%. Analysis condition A: Retention time=1.49 min; ESI-MS(+) m/z [M+3H]³⁺: 947.1.

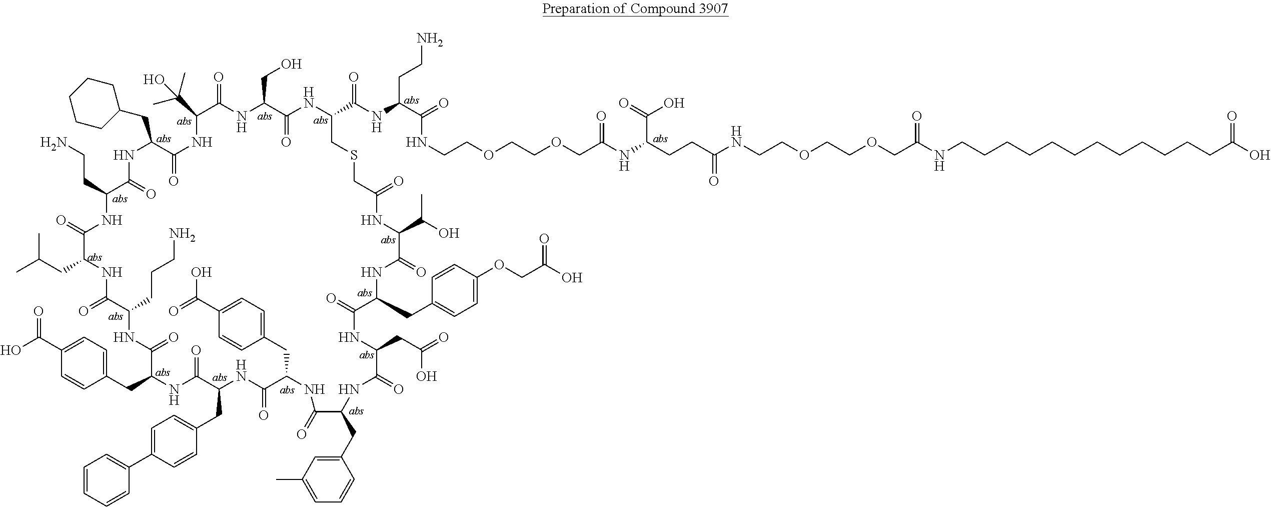 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(=O)NCCOCCOCC(=O)N[C@@H](CCC(=O)NCCOCCOCC(=O)NCCCCCCCCCCCCC(=O)O)C(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 3907 was prepared on a 50 μmol scale. The yield of the product was 7.5 mg, and its estimated purity by LCMS analysis was 100%. Analysis condition A: Retention time=1.48 min; ESI-MS(+) m/z [M+2H]²⁺: 1390.1.

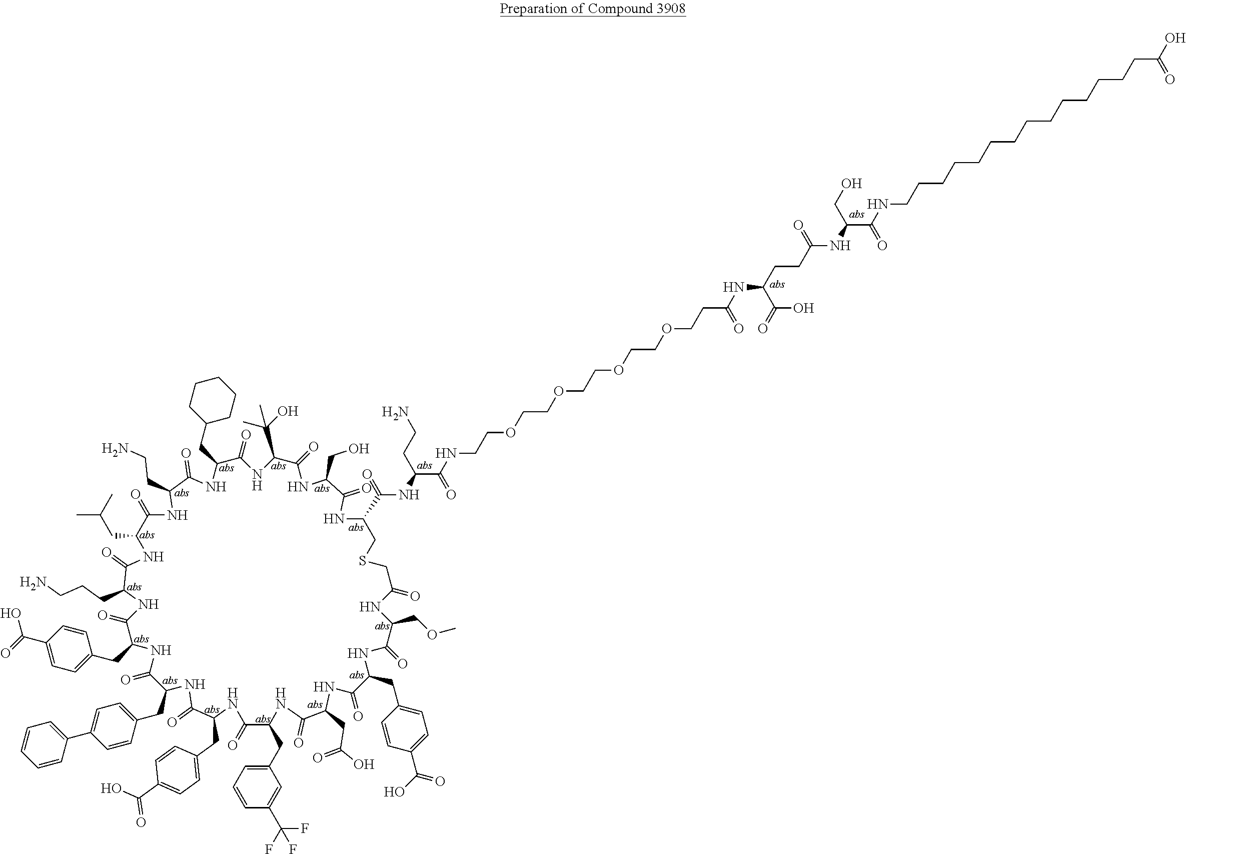 [SMILES: COC[C@@H]1NC(=O)CSC[C@@H](C(=O)N[C@@H](CCN)C(=O)NCCOCCOCCOCCOCCC(=O)N[C@@H](CCC(=O)N[C@@H](CO)C(=O)NCCCCCCCCCCCCCCC(=O)O)C(=O)O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC1=O]

Compound 3908 was prepared on a 50 μmol scale. The yield of the product was 10.9 mg, and its estimated purity by LCMS analysis was 97.6%. Analysis condition 1: Retention time=1.56 min; ESI-MS(+) m/z [M+2H]²⁺: 1438.1.

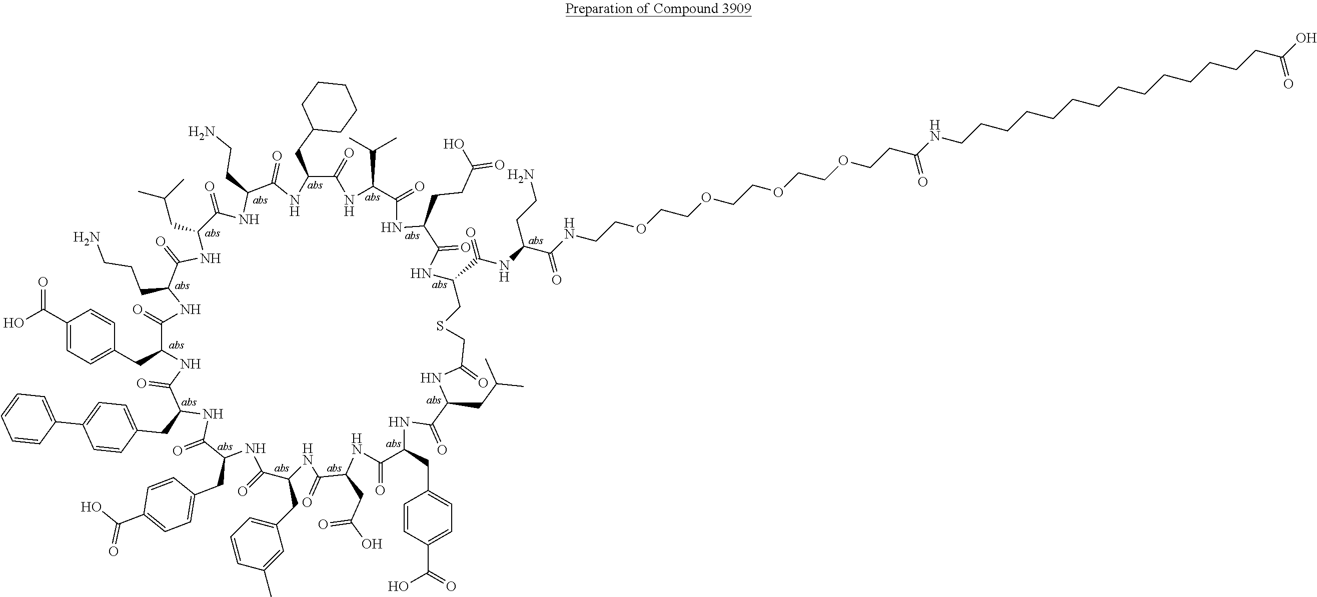

Compound 3909 was prepared on a 50 μmol scale. The yield of the product was 13.5 mg, and its estimated purity by LCMS analysis was 96.2%. Analysis condition 1: Retention time=1.44 min; ESI-MS(+) m/z [M+2H]²⁺: 1322.2.

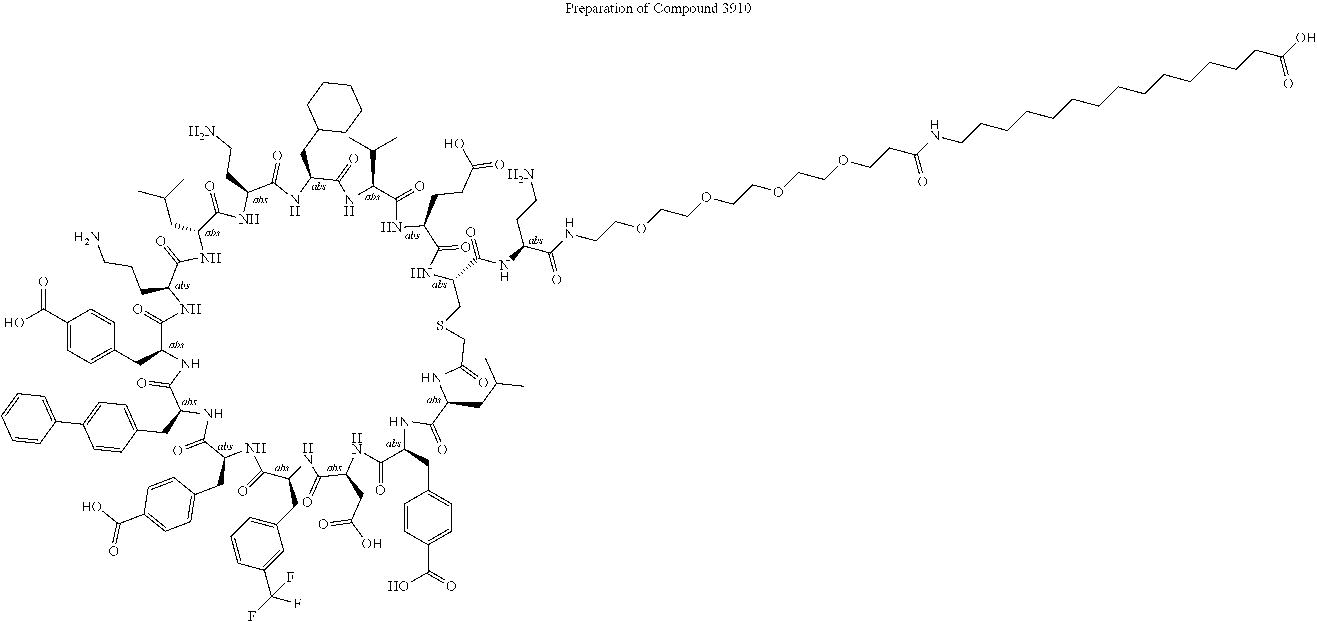

Compound 3910 was prepared on a 50 μmol scale. The yield of the product was 4.3 mg, and its estimated purity by LCMS analysis was 97.5%. Analysis condition 1: Retention time=1.79 min; ESI-MS(+) m/z [M+2H]²⁺: 1349.2.

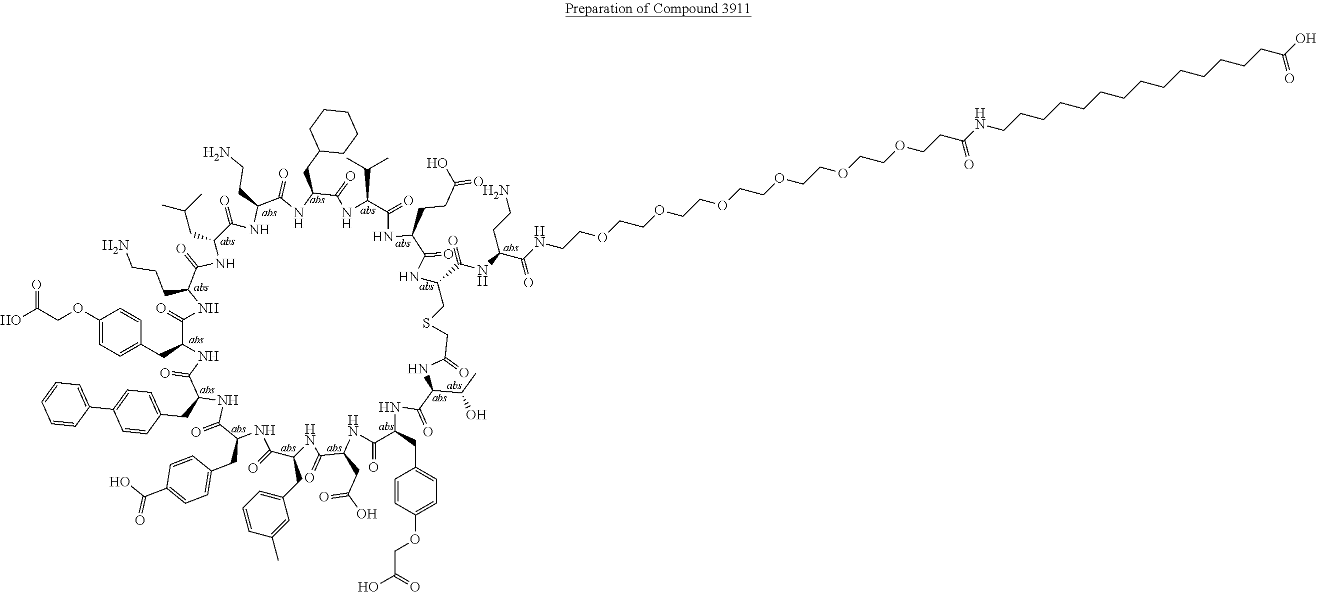

Compound 3911 was prepared on a 50 μmol scale. The yield of the product was 6.4 mg, and its estimated purity by LCMS analysis was 99.1%. Analysis condition 2: Retention time=1.95 min; ESI-MS(+) m/z [M+2H]²⁺: 1390.3.

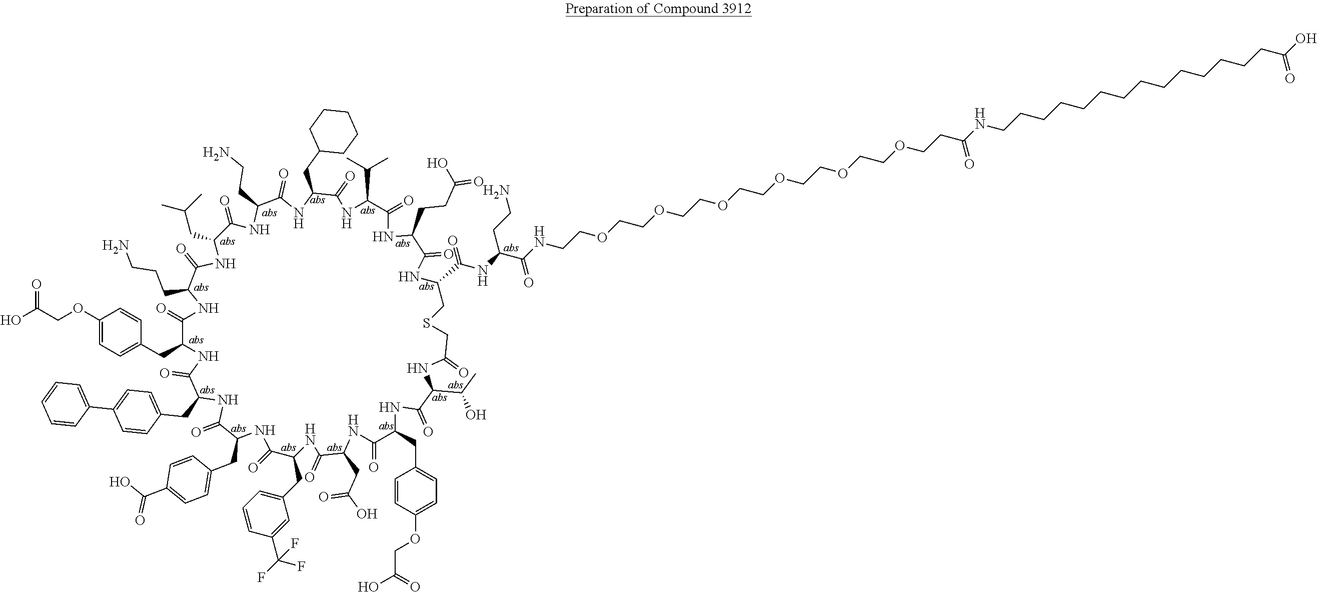

Compound 3912 was prepared on a 50 μmol scale. The yield of the product was 5.8 mg, and its estimated purity by LCMS analysis was 97%. Analysis condition 2: Retention time=2 min; ESI-MS(+) m/z [M+2H]²⁺: 1417.3.

### Biological Activity

### Example 2: Jurkat-PD-1 Cell Binding High-Content Screening Assay (CBA)

Phycoerythrin (PE) was covalently linked to the Ig epitope tag of human PD-L1-Ig and fluorescently-labeled PD-L1-Ig was used for binding studies with a Jurkat cell line over-expressing human PD-1 (Jurkat-PD-1). Briefly, 8×10³ Jurkat-hPD-1 cells were seeded into 384 well plates in 20 μl of DMEM supplemented with 10% fetal calf serum. 100 nl of compound was added to cells followed by incubation at 37° C. for 2 h. Then, 5 μl of PE-labeled PD-L1-Ig (20 nM final), diluted in DMEM supplemented with 10% fetal calf serum. After 1 hour incubation, cells were fixed with 4% paraformaldehyde in dPBS containing 10 μg/ml Hoechst 33342 and then washed 3× in 100 μl dPBS. Data was collected and processed using a Cell Insight NXT High Content Imager and associated software. Results of the CBA are reported in Table 3.

TABLE 3

| Compound Number | Jurkat hPD1-PDL1 IC50 (uM) |
|---|---|
| 1000 | 0.0055 |
| 1002 | 0.0060 |
| 1001 | 0.0072 |
| 1003 | 0.0148 |
| 1004 | 0.0026 |
| 1005 | 0.0011 |
| 1006 | 0.0092 |
| 1007 | 0.0079 |
| 1008 | 0.0105 |
| 1009 | 0.0086 |
| 1010 | 0.0063 |
| 1011 | 0.0088 |
| 1012 | 0.0050 |
| 1013 | 0.0119 |
| 1014 | 0.0055 |
| 1015 | 0.0079 |
| 1016 | 0.0060 |
| 1017 | 0.0064 |
| 1018 | 0.0084 |
| 1019 | 0.0068 |
| 1020 | 0.0083 |
| 1021 | 0.0083 |
| 1022 | 0.0057 |
| 1023 | 0.0079 |
| 1024 | 0.0066 |
| 1025 | 0.0108 |
| 1026 | 0.0087 |
| 1027 | 0.0171 |
| 1028 | 0.0065 |
| 1029 | 0.0074 |
| 1030 | 0.0043 |
| 1031 | 0.0068 |
| 1032 | 0.0061 |
| 1033 | 0.0087 |
| 1034 | 0.0066 |
| 1035 | 0.0090 |
| 1036 | 0.0068 |
| 1037 | 0.0076 |
| 1038 | 0.0208 |
| 1039 | 0.0119 |
| 1040 | 0.0529 |
| 1041 | 0.0151 |
| 1042 | 0.0153 |
| 1043 | 0.0156 |
| 1044 | 0.0061 |
| 1045 | 0.0110 |
| 1046 | 0.0162 |
| 1047 | 0.0132 |
| 1048 | 0.0183 |
| 1049 | 0.0150 |
| 1050 | 0.0209 |
| 1051 | 0.0123 |
| 1052 | 0.0288 |
| 1053 | 0.0089 |
| 1054 | 0.0313 |
| 1055 | 0.0190 |
| 1056 | 0.0368 |
| 1057 | 0.0159 |
| 1058 | 0.0572 |
| 1059 | 0.0486 |
| 1060 | 0.0898 |
| 1061 | 0.0562 |
| 1062 | 0.0109 |
| 1063 | 0.0118 |
| 1064 | 0.0163 |
| 1065 | 0.0135 |
| 1066 | 0.0139 |
| 1067 | 0.0285 |
| 1068 | 0.0104 |
| 1069 | 0.0417 |
| 1070 | 0.0104 |
| 1071 | 0.0425 |
| 1072 | 0.0142 |
| 1073 | 0.0152 |
| 1074 | 0.0695 |
| 1075 | 0.0346 |
| 1076 | 0.0441 |
| 1077 | 0.0467 |
| 1078 | 0.0849 |
| 1079 | 0.0556 |
| 1080 | 0.0357 |
| 1081 | 0.0285 |
| 1082 | 0.0247 |
| 1083 | 0.0647 |
| 1084 | 0.0389 |
| 1085 | 0.0262 |
| 1086 | 0.0210 |
| 1087 | 0.0396 |
| 1088 | 0.0250 |
| 1089 | 0.0331 |
| 1090 | 0.0389 |
| 1091 | 0.0315 |
| 1092 | 0.0373 |
| 1093 | 0.0118 |
| 1094 | 0.0172 |
| 1095 | 0.0157 |
| 1096 | 0.0157 |
| 1097 | 0.0149 |
| 1098 | 0.0022 |
| 1099 | 0.0138 |
| 1100 | 0.0088 |
| 1101 | 0.0696 |
| 1102 | 0.0505 |
| 1103 | 0.0291 |
| 1104 | 0.0403 |
| 1105 | 0.0230 |
| 1106 | 0.0265 |
| 1107 | 0.0358 |
| 1108 | 0.0270 |
| 1109 | 0.0376 |
| 1110 | 0.0330 |
| 1111 | 0.0642 |
| 1112 | 0.0182 |
| 1113 | 0.0302 |
| 1114 | 0.0610 |
| 1115 | 0.0260 |
| 1116 | 0.0376 |
| 1117 | 0.0484 |
| 1118 | 0.0307 |
| 1119 | 0.0575 |
| 1120 | 0.0409 |
| 1121 | 0.0274 |
| 1122 | 0.0424 |
| 1123 | 0.0484 |
| 1124 | 0.0382 |
| 1125 | 0.0356 |
| 1126 | 0.0740 |
| 1127 | 0.0256 |
| 1128 | 0.0287 |
| 1129 | 0.0374 |
| 1130 | 0.0310 |
| 1131 | 0.0143 |
| 1132 | 0.0359 |
| 1133 | 0.0302 |
| 1134 | 0.0289 |
| 1135 | 0.0811 |
| 1136 | 0.0337 |
| 1137 | 0.0186 |
| 1138 | 0.0189 |
| 1139 | 0.0113 |
| 1140 | 0.0315 |
| 1141 | 0.0410 |
| 1142 | 0.0549 |
| 1143 | 0.0909 |
| 1144 | 0.0632 |
| 1145 | 0.0261 |
| 1146 | 0.1056 |
| 1147 | 0.0373 |
| 1148 | 0.0317 |
| 1149 | 0.0197 |
| 1150 | 0.0335 |
| 1151 | 0.0167 |
| 1152 | 0.0344 |
| 1153 | 0.0435 |
| 1154 | 0.0215 |
| 1155 | 0.0176 |
| 1156 | 0.0345 |
| 1157 | 0.0146 |
| 1158 | 0.0166 |
| 1159 | 0.0334 |
| 1160 | 0.0210 |
| 1161 | 0.0253 |
| 1162 | 0.0208 |
| 1163 | 0.0551 |
| 1164 | 0.1216 |
| 1165 | 0.1043 |
| 1166 | 0.0280 |
| 1167 | 0.0801 |
| 1168 | 0.1475 |
| 1169 | 0.0287 |
| 1170 | 0.0379 |
| 1171 | 0.0280 |
| 1172 | 0.0250 |
| 1173 | 0.0374 |
| 1174 | 0.0166 |
| 1175 | 0.0199 |
| 1176 | 0.0072 |
| 1177 | 0.0083 |
| 1178 | 0.0097 |
| 1179 | 0.0103 |
| 1180 | 0.0184 |
| 1181 | 0.0117 |
| 1182 | 0.0437 |
| 1183 | 0.0306 |
| 1184 | 0.0515 |
| 1185 | 0.0442 |
| 1186 | 0.1029 |
| 1187 | 0.0628 |
| 1188 | 0.1481 |
| 1189 | 0.0236 |
| 1190 | 0.0248 |
| 1191 | 0.0197 |
| 1192 | 0.0455 |
| 1193 | 0.0103 |
| 1194 | 0.0148 |
| 1195 | 0.0137 |
| 1196 | 0.0096 |
| 1197 | 0.0148 |
| 1198 | 0.0151 |
| 1199 | 0.0133 |
| 1200 | 0.0156 |
| 1201 | 0.0076 |
| 1202 | 0.0167 |
| 1203 | 0.0374 |
| 1204 | 0.0092 |
| 1205 | 0.0200 |
| 1206 | 0.0198 |
| 1207 | 0.0285 |
| 1208 | 0.0269 |
| 1209 | 0.0352 |
| 1210 | 0.0222 |
| 1211 | 0.0156 |
| 1212 | 0.0332 |
| 1213 | 0.0328 |
| 1214 | 0.0137 |
| 1215 | 0.0138 |
| 1216 | 0.0165 |
| 1217 | 0.0258 |
| 1218 | 0.0329 |
| 1219 | 0.0371 |
| 1220 | 0.0173 |
| 1221 | 0.0360 |
| 1222 | 0.0407 |
| 1223 | 0.1354 |
| 1224 | 0.1409 |
| 1225 | 0.1707 |
| 1226 | 0.2774 |
| 1227 | 0.3043 |
| 1228 | 0.3068 |
| 1229 | 0.5178 |
| 1230 | 0.5373 |
| 1231 | 0.0358 |
| 1232 | 0.0171 |
| 1233 | 0.0221 |
| 1234 | 0.0398 |
| 1235 | 0.0321 |
| 1236 | 0.0357 |
| 1237 | 0.0706 |
| 1238 | 0.0393 |
| 1239 | 0.0302 |
| 1240 | 0.0206 |
| 1241 | 0.0184 |
| 1242 | 0.0333 |
| 1243 | 0.0117 |
| 1244 | 0.0162 |
| 1245 | 0.0351 |
| 1246 | 0.0154 |
| 1247 | 0.0176 |
| 1248 | 0.3673 |
| 1249 | 0.0817 |
| 1250 | 0.0404 |
| 1251 | 0.0380 |
| 1252 | 0.0415 |
| 1253 | 0.0311 |
| 1254 | 0.0090 |
| 1255 | 0.0102 |
| 1256 | 0.0099 |
| 1257 | 0.0200 |
| 1258 | 0.0446 |
| 1259 | 0.1526 |
| 1260 | 0.0149 |
| 1261 | 0.0093 |
| 1262 | 0.4614 |
| 1263 | 0.0056 |
| 1264 | 0.0036 |
| 1265 | 0.0065 |
| 1266 | 0.0140 |
| 1267 | 0.0152 |
| 1268 | 0.0132 |
| 1269 | 0.0114 |
| 1270 | 0.0484 |
| 1271 | 0.0199 |
| 1272 | 0.0742 |
| 1273 | 0.3306 |
| 1274 | 0.0098 |
| 1275 | 0.0475 |
| 1276 | 0.0499 |
| 1277 | 0.0144 |
| 1278 | 0.1403 |
| 1279 | 0.0090 |
| 1280 | 0.2427 |
| 1281 | 0.0047 |
| 1282 | 0.6310 |
| 1283 | 0.7307 |
| 1284 | 0.0136 |
| 1285 | 0.0064 |
| 1286 | 0.0056 |
| 1287 | 0.0082 |
| 1288 | 0.0103 |
| 1289 | 0.0109 |
| 1290 | 0.0125 |
| 1291 | 0.0147 |
| 1292 | 0.0180 |
| 1293 | 0.0191 |
| 1294 | 0.0196 |
| 1295 | 0.0222 |
| 1296 | 0.0240 |
| 1297 | 0.0246 |
| 1298 | 0.0288 |
| 1299 | 0.0304 |
| 1300 | 0.0312 |
| 1301 | 0.0319 |
| 1302 | 0.0319 |
| 1303 | 0.0322 |
| 1304 | 0.0373 |
| 1305 | 0.0434 |
| 1306 | 0.0489 |
| 1307 | 0.0496 |
| 1308 | 0.0597 |
| 1309 | 0.0634 |
| 1310 | 0.0646 |
| 1311 | 0.1030 |
| 1312 | 0.1046 |
| 1313 | 0.1093 |
| 1314 | 0.1096 |
| 2000 | 0.0143 |
| 2001 | 0.0324 |
| 2002 | 0.0043 |
| 2003 | 0.1358 |
| 2004 | 0.0186 |
| 2005 | 0.0116 |
| 2006 | 0.0077 |
| 2007 | 0.0137 |
| 2008 | 0.0196 |
| 2009 | 0.0172 |
| 2010 | 0.0153 |
| 2011 | 0.0248 |
| 2012 | 0.0460 |
| 2013 | 0.0245 |
| 2014 | 0.0330 |
| 2015 | 0.0198 |
| 2016 | 0.0176 |
| 2017 | 0.0193 |
| 2018 | 0.0404 |
| 2019 | 0.0499 |
| 2020 | 0.0105 |
| 2021 | 0.0308 |
| 2022 | 0.0595 |
| 2023 | 0.0102 |
| 2024 | 0.0130 |
| 2025 | 0.0205 |
| 2026 | 0.0102 |
| 2027 | 0.0150 |
| 2028 | 0.0137 |
| 2029 | 0.0080 |
| 2030 | 0.0072 |
| 2031 | 0.0404 |
| 2032 | 0.1467 |
| 2033 | 0.0292 |
| 2034 | 0.0155 |
| 2035 | 0.0145 |
| 2036 | 0.0144 |
| 2037 | 0.0169 |
| 2038 | 0.0387 |
| 2039 | 0.0194 |
| 2040 | 0.0379 |
| 2041 | 0.0082 |
| 2042 | 0.0050 |
| 2043 | 0.0063 |
| 2044 | 0.0066 |
| 2045 | 0.0072 |
| 2046 | 0.0097 |
| 2047 | 0.0134 |
| 2048 | 0.0124 |
| 2049 | 0.0169 |
| 2050 | 0.0108 |
| 2051 | 0.0326 |
| 2052 | 0.0147 |
| 2053 | 0.0069 |
| 2054 | 0.0129 |
| 2055 | 0.0079 |
| 2056 | 0.0126 |
| 2057 | 0.0080 |
| 2058 | 0.0047 |
| 2059 | 0.0040 |
| 2060 | 0.0043 |
| 2061 | 0.0039 |
| 2062 | 0.0070 |
| 2063 | 0.0049 |
| 2064 | 0.0154 |
| 2065 | 0.0048 |
| 2066 | 0.0060 |
| 2067 | 0.0532 |
| 2068 | 0.0133 |
| 2069 | 0.0149 |
| 2070 | 0.0116 |
| 2071 | 0.0048 |
| 2072 | 0.0085 |
| 2073 | 0.0089 |
| 2074 | 0.0055 |
| 2075 | 0.0143 |
| 2076 | 0.0084 |
| 2077 | 0.0063 |
| 2078 | 0.0039 |
| 2079 | 0.2447 |
| 2080 | 0.2184 |
| 2081 | 0.0117 |
| 2082 | 0.0227 |
| 2083 | 0.0083 |
| 2084 | 0.0126 |
| 2085 | 0.0086 |
| 2086 | 0.0102 |
| 2087 | 0.0151 |
| 2088 | 0.0148 |
| 2089 | 0.0438 |
| 2090 | 0.0184 |
| 2091 | 0.0261 |
| 2092 | 0.0251 |
| 2093 | 0.0232 |
| 2094 | 0.0066 |
| 2095 | 0.0095 |
| 2096 | 0.0365 |
| 2097 | 0.0388 |
| 2098 | 0.0534 |
| 2099 | 0.0180 |
| 2100 | 0.0139 |
| 2101 | 0.0162 |
| 2102 | 0.0291 |
| 2103 | 0.0699 |
| 2104 | 0.0223 |
| 2105 | 0.1129 |
| 2106 | 0.0159 |
| 2107 | 0.1165 |
| 2108 | 0.0210 |
| 2109 | 0.0222 |
| 2110 | 0.0241 |
| 2111 | 0.0064 |
| 2112 | 0.0055 |
| 2113 | 0.0087 |
| 2114 | 0.0100 |
| 2115 | 0.0293 |
| 2116 | 0.0535 |
| 2117 | 0.0263 |
| 2118 | 0.1259 |
| 2119 | 0.0103 |
| 2120 | 0.0854 |
| 2121 | 0.0083 |
| 2122 | 0.0109 |
| 2123 | 0.0141 |
| 2124 | 0.0173 |
| 2125 | 0.0164 |
| 2126 | 0.0357 |
| 2127 | 0.0284 |
| 2128 | 0.0106 |
| 2129 | 0.0060 |
| 2130 | 0.0157 |
| 2131 | 0.0058 |
| 2132 | 0.0115 |
| 2133 | 0.0588 |
| 2134 | 0.0385 |
| 2135 | 0.0688 |
| 2136 | 0.0031 |
| 2137 | 0.0091 |
| 2138 | 0.1090 |
| 2139 | 0.0211 |
| 2140 | 0.0116 |
| 2141 | 0.0109 |
| 2142 | 0.0091 |
| 2143 | 0.0126 |
| 2144 | 0.0073 |
| 2145 | 0.0021 |
| 2145 | 0.0132 |
| 2147 | 0.0172 |
| 2148 | 0.0254 |
| 2149 | 0.0551 |
| 2150 | 0.5652 |
| 2151 | 0.0406 |
| 2152 | 0.0736 |
| 2153 | 0.0234 |
| 2154 | 0.0092 |
| 2155 | 0.0194 |
| 2156 | 0.0098 |
| 2157 | 0.0978 |
| 2158 | 0.1286 |
| 2159 | 0.0961 |
| 2160 | 0.0426 |
| 2161 | 0.0129 |
| 2162 | 0.0129 |
| 2163 | 0.0930 |
| 2164 | 0.0333 |
| 2165 | 0.0218 |
| 2166 | 0.0118 |
| 2167 | 0.0094 |
| 2168 | 0.0267 |
| 2169 | 0.0143 |
| 2170 | 0.0079 |
| 2171 | 0.0046 |
| 2172 | 0.0306 |
| 2173 | 0.0550 |
| 2174 | 0.0113 |
| 2175 | 0.0108 |
| 2176 | 0.1054 |
| 2177 | 0.1438 |
| 2178 | 0.0064 |
| 2179 | 0.0220 |
| 2180 | 0.1193 |
| 2181 | 0.0177 |
| 2182 | 0.0156 |
| 2183 | 0.0133 |
| 2184 | 0.0103 |
| 2185 | 0.0096 |
| 2186 | 0.0140 |
| 2187 | 0.0313 |
| 2188 | 0.0094 |
| 2189 | 0.0182 |
| 2190 | 0.0196 |
| 2191 | 0.0200 |
| 2192 | 0.0077 |
| 2193 | 0.0217 |
| 2194 | 0.0129 |
| 2195 | 0.0118 |
| 2196 | 0.0244 |
| 2197 | 0.0151 |
| 2198 | 0.0481 |
| 2199 | 0.0500 |
| 2200 | 0.0288 |
| 2201 | 0.0784 |
| 2202 | 0.0259 |
| 2203 | 0.0417 |
| 2204 | 0.0106 |
| 2205 | 0.0125 |
| 2206 | 0.0075 |
| 2207 | 0.0076 |
| 2208 | 0.0140 |
| 2209 | 0.0455 |
| 2210 | 0.0088 |
| 2211 | 0.0090 |
| 2212 | 0.0100 |
| 2213 | 0.0110 |
| 2214 | 0.0080 |
| 2215 | 0.0128 |
| 2216 | 0.0367 |
| 2217 | 0.0279 |
| 2218 | 0.0071 |
| 2219 | 0.0047 |
| 2220 | 0.0123 |
| 2222 | 0.0176 |
| 2223 | 0.2802 |
| 2224 | 0.0262 |
| 2226 | 0.0278 |
| 2227 | 0.2043 |
| 2228 | 0.0781 |
| 2229 | 0.0006 |
| 2230 | 0.0022 |
| 2231 | 0.0152 |
| 2232 | 0.2375 |
| 2233 | 0.0050 |
| 2234 | 0.0026 |
| 2235 | 0.0009 |
| 2236 | 0.1852 |
| 2237 | 0.0026 |
| 2238 | 0.0112 |
| 2239 | 0.0016 |
| 2240 | 0.0073 |
| 2241 | 0.0037 |
| 2242 | 0.0040 |
| 2243 | 0.0017 |
| 2244 | 0.0006 |
| 2245 | 0.0012 |
| 2246 | 0.0006 |
| 2247 | 0.0013 |
| 2248 | 0.0051 |
| 2249 | 0.0160 |
| 2250 | 0.0180 |
| 2251 | 0.0105 |
| 2252 | 0.0135 |
| 2253 | 0.0091 |
| 2254 | 0.0101 |
| 2255 | 0.0114 |
| 2256 | 0.0136 |
| 2257 | 0.0111 |
| 2258 | 0.0489 |
| 2259 | 0.0179 |
| 2260 | 0.0094 |
| 2261 | 0.2071 |
| 2262 | 0.0083 |
| 2263 | 0.0382 |
| 2264 | 0.0131 |
| 2501 | 0.0284 |
| 2502 | 0.0212 |
| 2503 | 0.0399 |
| 2504 | 0.0374 |
| 2505 | 0.0209 |
| 2506 | 0.0324 |
| 2507 | 0.0721 |
| 2508 | 0.1322 |
| 2509 | 0.0598 |
| 2510 | 0.0650 |
| 2511 | 0.0501 |
| 2512 | 0.0334 |
| 2513 | 0.0813 |
| 2514 | 0.0497 |
| 2515 | 0.0404 |
| 2516 | 0.1258 |
| 2517 | 0.2738 |
| 2518 | 0.2071 |
| 2519 | 0.1521 |
| 2520 | 0.2330 |
| 2521 | 0.0891 |
| 2522 | 0.3405 |
| 2523 | 0.0076 |
| 2524 | 0.0145 |
| 2525 | 0.0165 |
| 2526 | 0.0072 |
| 2527 | 0.0068 |
| 2528 | 0.0078 |
| 2529 | 0.0127 |
| 2530 | 0.0109 |
| 2531 | 0.0222 |
| 2532 | 0.0346 |
| 2533 | 0.0238 |
| 2534 | 0.0063 |
| 2535 | 0.0064 |
| 2536 | 0.0095 |
| 2537 | 0.0189 |
| 2538 | 0.0136 |
| 2539 | 0.0053 |
| 2540 | 0.0070 |
| 2541 | 0.0076 |
| 2542 | 0.0049 |
| 2543 | 0.0095 |
| 2544 | 0.0107 |
| 2545 | 0.0074 |
| 2546 | 0.0133 |
| 2547 | 0.0319 |
| 2548 | 0.0116 |
| 2549 | 0.0094 |
| 2550 | 0.0080 |
| 2551 | 0.0112 |
| 2552 | 0.0099 |
| 2553 | 0.0113 |
| 2554 | 0.0115 |
| 2555 | 0.0241 |
| 2556 | 0.0437 |
| 2557 | 0.0121 |
| 2558 | 0.0107 |
| 2559 | 0.0113 |
| 2560 | 0.0170 |
| 2561 | 0.0146 |
| 2562 | 0.0392 |
| 2563 | 0.0293 |
| 2564 | 0.0100 |
| 2565 | 0.0054 |
| 2566 | 0.0094 |
| 2567 | 0.0053 |
| 2568 | 0.0127 |
| 2569 | 0.0802 |
| 2570 | 0.0319 |
| 2571 | 0.0130 |
| 2572 | 0.0216 |
| 2573 | 0.0476 |
| 2574 | 0.0283 |
| 2575 | 0.0102 |
| 2576 | 0.0104 |
| 2577 | 0.0340 |
| 2578 | 0.0107 |
| 2579 | 0.0118 |
| 2580 | 0.0077 |
| 2581 | 0.0214 |
| 2582 | 0.0095 |
| 2583 | 0.0323 |
| 2584 | 0.0179 |
| 2585 | 0.0270 |
| 2586 | 0.0222 |
| 2587 | 0.0088 |
| 2588 | 0.0053 |
| 2589 | 0.0136 |
| 2590 | 0.0095 |
| 2591 | 0.0095 |
| 2592 | 0.0108 |
| 2593 | 0.0184 |
| 2594 | 0.0116 |
| 2595 | 0.0341 |
| 2596 | 0.0096 |
| 2597 | 0.0089 |
| 2598 | 0.0106 |
| 2599 | 0.0082 |
| 2600 | 0.0103 |
| 2601 | 0.0182 |
| 2602 | 0.0184 |
| 2603 | 0.0071 |
| 2604 | 0.0132 |
| 2605 | 0.0056 |
| 2606 | 0.0073 |
| 2607 | 0.0297 |
| 2608 | 0.0317 |
| 2609 | 0.0131 |
| 2610 | 0.0100 |
| 2611 | 0.0241 |
| 2612 | 0.0186 |
| 2613 | 0.0262 |
| 2614 | 0.0142 |
| 2615 | 0.0085 |
| 2616 | 0.0161 |
| 2617 | 0.0063 |
| 2618 | 0.0125 |
| 2619 | 0.0244 |
| 2620 | 0.0281 |
| 2621 | 0.0168 |
| 2622 | 0.0306 |
| 2623 | 0.0275 |
| 2624 | 0.0345 |
| 2625 | 0.0167 |
| 2626 | 0.0119 |
| 2627 | 0.0141 |
| 2628 | 0.0151 |
| 2629 | 0.0086 |
| 2630 | 0.0099 |
| 2631 | 0.0141 |
| 2632 | 0.0124 |
| 2633 | 0.0164 |
| 2634 | 0.0141 |
| 2635 | 0.0103 |
| 2636 | 0.0225 |
| 2637 | 0.0155 |
| 2638 | 0.0190 |
| 2639 | 0.0185 |
| 2640 | 0.0081 |
| 2641 | 0.0277 |
| 2642 | 0.0180 |
| 2643 | 0.0072 |
| 2644 | 0.0094 |
| 2645 | 0.0154 |
| 2646 | 0.0132 |
| 2647 | 0.0077 |
| 2648 | 0.0089 |
| 2649 | 0.0111 |
| 2650 | 0.0115 |
| 2651 | 0.0097 |
| 2652 | 0.0061 |
| 2653 | 0.0112 |
| 2654 | 0.0052 |
| 2655 | 0.0094 |
| 2656 | 0.0082 |
| 2657 | 0.0144 |
| 2658 | 0.0180 |
| 2659 | 0.0115 |
| 2660 | 0.0114 |
| 2661 | 0.0109 |
| 2662 | 0.0110 |
| 2663 | 0.0176 |
| 2664 | 0.0117 |
| 2665 | 0.0222 |
| 2666 | 0.0180 |
| 2667 | 0.0096 |
| 2668 | 0.0108 |
| 2669 | 0.0115 |
| 2670 | 0.0108 |
| 2671 | 0.0141 |
| 2672 | 0.0227 |
| 2673 | 0.0225 |
| 2674 | 0.0120 |
| 2675 | 0.0241 |
| 2676 | 0.0102 |
| 2677 | 0.0180 |
| 2678 | 0.0152 |
| 2679 | 0.0269 |
| 2680 | 0.0132 |
| 2681 | 0.0130 |
| 2682 | 0.0174 |
| 2683 | 0.0149 |
| 2684 | 0.0363 |
| 2685 | 0.0116 |
| 2686 | 0.0161 |
| 2687 | 0.0100 |
| 2688 | 0.0156 |
| 2689 | 0.0095 |
| 2690 | 0.0089 |
| 2691 | 0.0150 |
| 2692 | 0.0063 |
| 2693 | 0.0130 |
| 2694 | 0.0099 |
| 2695 | 0.0051 |
| 2696 | 0.0191 |
| 2697 | 0.0049 |
| 2698 | 0.0110 |
| 2699 | 0.0054 |
| 2700 | 0.0179 |
| 2701 | 0.0067 |
| 2702 | 0.0257 |
| 2703 | 0.0193 |
| 2704 | 0.0267 |
| 2705 | 0.0083 |
| 2706 | 0.0184 |
| 2707 | 0.0208 |
| 2708 | 0.0628 |
| 2709 | 0.0084 |
| 2710 | 0.0186 |
| 2711 | 0.0104 |
| 2712 | 0.0206 |
| 2713 | 0.0077 |
| 2714 | 0.4063 |
| 2715 | 0.0089 |
| 2716 | 0.0243 |
| 2717 | 0.0171 |
| 2718 | 0.0313 |
| 2719 | 0.0074 |
| 2720 | 0.1346 |
| 2721 | 0.0224 |
| 2722 | 0.0343 |
| 2723 | 0.0269 |
| 2724 | 0.0077 |
| 2725 | 0.0299 |
| 2726 | 0.0241 |
| 2727 | 0.0301 |
| 2728 | 0.0174 |
| 2729 | 0.0129 |
| 2730 | 0.0155 |
| 2731 | 0.0186 |
| 2732 | 0.0212 |
| 2733 | 0.0422 |
| 2734 | 0.0272 |
| 2735 | 0.0724 |
| 2736 | 0.1141 |
| 2737 | 0.0288 |
| 2738 | 0.0632 |
| 2739 | 0.0189 |
| 2740 | 0.0197 |
| 2741 | 0.0543 |
| 2742 | 0.1331 |
| 2743 | 0.0976 |
| 2744 | 0.0725 |
| 2745 | 0.2623 |
| 2746 | 0.0486 |
| 2747 | 0.0450 |
| 2748 | 0.1472 |
| 2749 | 0.0400 |
| 2750 | 0.0091 |
| 2751 | 0.0125 |
| 2752 | 0.1310 |
| 2753 | 0.1132 |
| 2754 | 0.0539 |
| 2755 | 0.0305 |
| 2756 | 0.0407 |
| 2757 | 0.1771 |
| 2758 | 0.0991 |
| 2759 | 0.0711 |
| 2760 | 0.0381 |
| 2761 | 0.1544 |
| 2762 | 0.1974 |
| 2763 | 0.0549 |
| 2764 | 0.0689 |
| 2765 | 0.0624 |
| 2766 | 0.0559 |
| 2767 | 0.0427 |
| 2768 | 0.0552 |
| 2769 | 0.0607 |
| 2770 | 0.0378 |
| 2771 | 0.0395 |
| 2772 | 0.0677 |
| 2773 | 0.0220 |
| 2774 | 0.0719 |
| 2775 | 0.1729 |
| 2776 | 0.0541 |
| 2777 | 0.0201 |
| 2778 | 0.0089 |
| 2779 | 0.0181 |
| 2780 | 0.0067 |
| 2781 | 0.0743 |
| 2782 | 0.3364 |
| 2783 | 0.3681 |
| 2784 | 0.0218 |
| 2785 | 0.0161 |
| 2786 | 0.0270 |
| 2787 | 0.0283 |
| 2788 | 0.0223 |
| 2789 | 0.0238 |
| 2790 | 0.0711 |
| 2791 | 0.0367 |
| 2792 | 0.0757 |
| 2793 | 0.0216 |
| 2794 | 0.0280 |
| 2795 | 0.0634 |
| 2796 | 0.0785 |
| 2797 | 0.0118 |
| 2798 | 0.0071 |
| 2799 | 0.0200 |
| 2800 | 0.0846 |
| 2801 | 0.3274 |
| 2802 | 0.0154 |
| 2803 | 0.0739 |
| 2804 | 0.0510 |
| 2805 | 0.0099 |
| 2806 | 0.0130 |
| 2807 | 0.0165 |
| 2808 | 0.2165 |
| 2809 | 0.1799 |
| 2810 | 0.0184 |
| 2811 | 0.0341 |
| 2812 | 0.0240 |
| 2813 | 0.1813 |
| 2814 | 0.0236 |
| 2815 | 0.0182 |
| 2816 | 0.0749 |
| 2817 | 0.0471 |
| 2818 | 0.0233 |
| 2819 | 0.0210 |
| 2820 | 0.0239 |
| 2821 | 0.0109 |
| 2822 | 0.0219 |
| 2823 | 0.0164 |
| 2824 | 0.0255 |
| 2825 | 0.2011 |
| 2826 | 0.0622 |
| 2827 | 0.0131 |
| 2828 | 0.0774 |
| 2829 | 0.1619 |
| 2830 | 0.0260 |
| 2831 | 0.0432 |
| 2832 | 0.0480 |
| 2833 | 0.0052 |
| 2834 | 0.0202 |
| 2835 | 0.0189 |
| 2836 | 0.0164 |
| 2837 | 0.0430 |
| 2838 | 0.0302 |
| 2839 | 0.1862 |
| 2840 | 0.0181 |
| 2841 | 0.0123 |
| 2842 | 0.0111 |
| 2843 | 0.0092 |
| 2844 | 0.0255 |
| 2845 | 0.0142 |
| 2846 | 0.0128 |
| 2847 | 0.0205 |
| 2848 | 0.0149 |
| 2849 | 0.0050 |
| 2850 | 0.0304 |
| 2851 | 0.0213 |
| 2852 | 0.0210 |
| 2853 | 0.0655 |
| 2854 | 0.0168 |
| 2855 | 0.0196 |
| 2856 | 0.0197 |
| 2857 | 0.0350 |
| 2858 | 0.0119 |
| 2859 | 0.0078 |
| 2860 | 0.0112 |
| 2861 | 0.0051 |
| 2862 | 0.2277 |
| 2863 | 0.0087 |
| 2864 | 0.0033 |
| 2865 | 0.0194 |
| 2866 | 0.1006 |
| 2867 | 0.0111 |
| 2868 | 0.4667 |
| 2869 | 0.0070 |
| 2870 | 0.1149 |
| 2871 | 0.0169 |
| 2872 | 0.0838 |
| 2873 | 0.0345 |
| 2874 | 0.2742 |
| 2875 | 0.0083 |
| 2876 | 0.2120 |
| 2877 | 0.0015 |
| 2878 | 0.0024 |
| 2879 | 0.0027 |
| 2880 | 0.0017 |
| 2881 | 0.0011 |
| 2882 | 0.0016 |
| 2883 | 0.0049 |
| 2884 | 0.1334 |
| 2885 | 2.6935 |
| 2886 | 0.0100 |
| 2887 | 0.0184 |
| 2888 | 0.0382 |
| 2889 | 0.0524 |
| 2890 | 0.0311 |
| 2891 | 0.0026 |
| 2892 | 0.0262 |
| 2893 | 0.0195 |
| 2894 | 0.2586 |
| 2895 | 0.0114 |
| 2896 | 0.0041 |
| 2897 | 0.0016 |
| 2898 | 0.0055 |
| 2899 | 0.0054 |
| 2900 | 0.0062 |
| 2901 | 0.0241 |
| 2902 | 0.2821 |
| 2903 | 0.0277 |
| 2904 | 0.5176 |
| 2905 | 0.0706 |
| 2906 | 0.0977 |
| 2907 | 0.0418 |
| 2908 | 0.5412 |
| 2909 | 0.0182 |
| 2910 | 0.1148 |
| 2911 | 0.0349 |
| 2912 | 0.2402 |
| 2913 | 0.6309 |
| 2914 | 0.0240 |
| 2915 | 0.1760 |
| 2916 | 0.0253 |
| 2917 | 0.1354 |
| 2918 | 0.0224 |
| 2919 | 0.3725 |
| 2920 | 0.0257 |
| 2921 | 0.0205 |
| 2922 | 0.4230 |
| 2923 | 0.0225 |
| 2924 | 0.0168 |
| 2925 | 0.0366 |
| 2926 | 0.0329 |
| 2927 | 0.2437 |
| 2928 | 0.0448 |
| 2929 | 0.3686 |
| 2930 | 0.0287 |
| 2931 | 0.0263 |
| 2932 | 0.0330 |
| 2933 | 0.0265 |
| 2934 | 0.0390 |
| 2935 | 0.0424 |
| 2936 | 0.0283 |
| 2937 | 0.0117 |
| 2938 | 0.0268 |
| 2939 | 0.0352 |
| 2940 | 0.0143 |
| 2941 | 0.0278 |
| 2942 | 0.0437 |
| 2943 | 0.0758 |
| 2944 | 0.0550 |
| 2945 | 0.0207 |
| 2946 | 0.1202 |
| 2947 | 0.0644 |
| 2948 | 0.3522 |
| 2949 | 0.3054 |
| 2950 | 0.0524 |
| 2951 | 0.3344 |
| 2952 | 0.1379 |
| 2953 | 0.0493 |
| 2954 | 0.2636 |
| 3000 | 0.0234 |
| 3001 | 0.0358 |
| 3002 | 0.0219 |
| 3003 | 0.0449 |
| 3004 | 0.0069 |
| 3005 | 0.0058 |
| 3006 | 0.1292 |
| 3007 | 0.1797 |
| 3008 | 0.5518 |
| 3009 | 0.1618 |
| 3010 | 0.1070 |
| 3011 | 0.2926 |
| 3012 | 0.3204 |
| 3013 | 0.0231 |
| 3014 | 0.4550 |
| 3015 | 0.0135 |
| 3016 | 0.0945 |
| 3017 | 0.0103 |
| 3018 | 0.1341 |
| 3019 | 0.3359 |
| 3020 | 0.0120 |
| 3021 | 0.1304 |
| 3022 | 0.0073 |
| 3023 | 0.1836 |
| 3024 | 0.8912 |
| 3025 | 0.0805 |
| 3026 | 0.0100 |
| 3027 | 0.0095 |
| 3028 | 0.0164 |
| 3029 | 0.0079 |
| 3030 | 0.0121 |
| 3031 | 0.0034 |
| 3032 | 0.0265 |
| 3033 | 0.0113 |
| 3034 | 0.1087 |
| 3035 | 0.0107 |
| 3036 | 0.0049 |
| 3037 | 0.0165 |
| 3038 | 0.0113 |
| 3039 | 0.0105 |
| 3040 | 0.0150 |
| 3041 | 0.0127 |
| 3042 | 0.0115 |
| 3043 | 0.0154 |
| 3044 | 0.0212 |
| 3045 | 0.0064 |
| 3046 | 0.0131 |
| 3047 | 0.0133 |
| 3048 | 0.0232 |
| 3049 | 0.0395 |
| 3050 | 0.0344 |
| 3051 | 0.0176 |
| 3052 | 0.0101 |
| 3053 | 0.0088 |
| 3054 | 0.0146 |
| 3055 | 0.0190 |
| 3056 | 0.0177 |
| 3057 | 0.0539 |
| 3058 | 0.0130 |
| 3059 | 0.0122 |
| 3060 | 0.0178 |
| 3061 | 0.0175 |
| 3062 | 0.0330 |
| 3063 | 0.0134 |
| 3064 | 0.0321 |
| 3065 | 0.0192 |
| 3066 | 0.0171 |
| 3067 | 0.0410 |
| 3068 | 0.0441 |
| 3069 | 0.0133 |
| 3070 | 0.0051 |
| 3071 | 0.0172 |
| 3072 | 0.0135 |
| 3073 | 0.0172 |
| 3074 | 0.0277 |
| 3075 | 0.0125 |
| 3076 | 0.0237 |
| 3077 | 0.0095 |
| 3078 | 0.0129 |
| 3079 | 0.0267 |
| 3080 | 0.0300 |
| 3081 | 0.0207 |
| 3082 | 0.0113 |
| 3083 | 0.0262 |
| 3084 | 0.1081 |
| 3085 | 0.0120 |
| 3086 | 0.0347 |
| 3087 | 0.0181 |
| 3088 | 0.0170 |
| 3089 | 0.0392 |
| 3090 | 0.0998 |
| 3091 | 0.0269 |
| 3092 | 0.0220 |
| 3093 | 0.0501 |
| 3094 | 0.2371 |
| 3095 | 0.0101 |
| 3096 | 0.0121 |
| 3097 | 0.0228 |
| 3098 | 0.0256 |
| 3099 | 0.0191 |
| 3100 | 0.0182 |
| 3101 | 0.0100 |
| 3102 | 0.0080 |
| 3103 | 0.0452 |
| 3104 | 0.0171 |
| 3105 | 0.0282 |
| 3106 | 0.0146 |
| 3107 | 0.0170 |
| 3108 | 0.0244 |
| 3109 | 0.0158 |
| 3110 | 0.0165 |
| 3111 | 0.0346 |
| 3112 | 0.1603 |
| 3113 | 0.1600 |
| 3114 | 0.0934 |
| 3115 | 0.2050 |
| 3116 | 0.0434 |
| 3117 | 0.1596 |
| 3118 | 0.0304 |
| 3119 | 0.0246 |
| 3120 | 0.0248 |
| 3121 | 0.0104 |
| 3122 | 0.0193 |
| 3123 | 0.0829 |
| 3124 | 0.2815 |
| 3125 | 0.0291 |
| 3126 | 0.0686 |
| 3127 | 0.3076 |
| 3128 | 0.0155 |
| 3129 | 0.0534 |
| 3130 | 0.0165 |
| 3131 | 0.0193 |
| 3132 | 0.0099 |
| 3133 | 0.0453 |
| 3134 | 0.0059 |
| 3135 | 0.0696 |
| 3136 | 0.0152 |
| 3137 | 0.0887 |
| 3138 | 0.0469 |
| 3139 | 0.0334 |
| 3140 | 0.0166 |
| 3141 | 0.0307 |
| 3142 | 0.0362 |
| 3143 | 0.0292 |
| 3144 | 0.0245 |
| 3145 | 0.0155 |
| 3146 | 0.0078 |
| 3147 | 0.0297 |
| 3148 | 0.0567 |
| 3149 | 0.0465 |
| 3150 | 0.0587 |
| 3151 | 0.0341 |
| 3152 | 0.0467 |
| 3153 | 0.0120 |
| 3154 | 0.0149 |
| 3155 | 0.0446 |
| 3156 | 0.0345 |
| 3157 | 0.0918 |
| 3158 | 0.1070 |
| 3159 | 0.0426 |
| 3160 | 0.0375 |
| 3161 | 0.0389 |
| 3162 | 0.0263 |
| 3163 | 0.0223 |
| 3164 | 0.0299 |
| 3165 | 0.0306 |
| 3166 | 0.0340 |
| 3167 | 0.0590 |
| 3168 | 0.0736 |
| 3169 | 0.0208 |
| 3170 | 0.0175 |
| 3171 | 0.0061 |
| 3172 | 0.0107 |
| 3173 | 0.0212 |
| 3174 | 0.0113 |
| 3175 | 0.0177 |
| 3176 | 0.0334 |
| 3177 | 0.0273 |
| 3178 | 0.0148 |
| 3179 | 0.0161 |
| 3180 | 0.0168 |
| 3181 | 0.0076 |
| 3182 | 0.0459 |
| 3183 | 0.8212 |
| 3184 | 0.0101 |
| 3185 | 0.0176 |
| 3186 | 0.0370 |
| 3187 | 0.0097 |
| 3188 | 0.3018 |
| 3189 | 0.0217 |
| 3190 | 0.0204 |
| 3191 | 0.0114 |
| 3192 | 0.0182 |
| 3193 | 0.0144 |
| 3194 | 0.0313 |
| 3195 | 0.0573 |
| 3196 | 0.0116 |
| 3197 | 0.0077 |
| 3198 | 0.0040 |
| 3199 | 0.0055 |
| 3200 | 0.0073 |
| 3201 | 0.0341 |
| 3202 | 0.0193 |
| 3203 | 0.0284 |
| 3204 | 0.0127 |
| 3205 | 0.0124 |
| 3206 | 0.0245 |
| 3207 | 0.0174 |
| 3208 | 0.6027 |
| 3209 | 0.0171 |
| 3210 | 0.0080 |
| 3211 | 0.0183 |
| 3212 | 0.0137 |
| 3213 | 0.0135 |
| 3214 | 0.0228 |
| 3215 | 0.0253 |
| 3216 | 0.0352 |
| 3217 | 0.0179 |
| 3218 | 0.0269 |
| 3219 | 0.0096 |
| 3220 | 0.0115 |
| 3221 | 0.0147 |
| 3222 | 0.0150 |
| 3223 | 0.0087 |
| 3224 | 0.0090 |
| 3225 | 0.0117 |
| 3226 | 0.0089 |
| 3227 | 0.0061 |
| 3228 | 0.0098 |
| 3229 | 0.0069 |
| 3230 | 0.0089 |
| 3231 | 0.0077 |
| 3232 | 0.0101 |
| 3233 | 0.0055 |
| 3234 | 0.0073 |
| 3235 | 0.0057 |
| 3236 | 0.0039 |
| 3237 | 0.1589 |
| 3238 | 0.0117 |
| 3239 | 0.0114 |
| 3240 | 0.0086 |
| 3241 | 0.0117 |
| 3242 | 0.6998 |
| 3243 | 0.0109 |
| 3244 | 0.0109 |
| 3245 | 0.0110 |
| 3246 | 0.0265 |
| 3247 | 0.0148 |
| 3248 | 0.0036 |
| 3249 | 0.0256 |
| 3250 | 0.0112 |
| 3251 | 0.0057 |
| 3252 | 0.0074 |
| 3253 | 0.0088 |
| 3254 | 0.0109 |
| 3255 | 0.0132 |
| 3256 | 0.0040 |
| 3257 | 0.0053 |
| 3258 | 0.0373 |
| 3259 | 0.0109 |
| 3260 | 0.0122 |
| 3261 | 0.0139 |
| 3262 | 0.0067 |
| 3263 | 0.0553 |
| 3264 | 0.0126 |
| 3265 | 0.0072 |
| 3266 | 0.0092 |
| 3267 | 0.0163 |
| 3268 | 0.0080 |
| 3269 | 0.0655 |
| 3270 | 0.0320 |
| 3271 | 0.0316 |
| 3272 | 0.0298 |
| 3273 | 0.0275 |
| 3274 | 0.4107 |
| 3275 | 0.4110 |
| 3276 | 0.0098 |
| 3277 | 0.0105 |
| 3278 | 0.0087 |
| 3279 | 0.0467 |
| 3280 | 0.0422 |
| 3281 | 0.0987 |
| 3282 | 0.1192 |
| 3283 | 0.0138 |
| 3284 | 0.0102 |
| 3285 | 0.0093 |
| 3286 | 0.0083 |
| 3287 | 0.0108 |
| 3288 | 0.0137 |
| 3289 | 0.0130 |
| 3290 | 0.0395 |
| 3291 | 0.0061 |
| 3292 | 0.0120 |
| 3293 | 0.0050 |
| 3294 | 0.0155 |
| 3295 | 0.0125 |
| 3296 | 0.0203 |
| 3297 | 0.0295 |
| 3298 | 0.0254 |
| 3299 | 0.0234 |
| 3300 | 0.0598 |
| 3301 | 0.0174 |
| 3302 | 0.0141 |
| 3303 | 0.0151 |
| 3304 | 0.0142 |
| 3305 | 0.0065 |
| 3306 | 0.0079 |
| 3307 | 0.0074 |
| 3308 | 0.0057 |
| 3309 | 0.0185 |
| 3310 | 0.0116 |
| 3311 | 0.0044 |
| 3312 | 0.0044 |
| 3313 | 0.0163 |
| 3314 | 0.0194 |
| 3315 | 0.0234 |
| 3316 | 0.0062 |
| 3317 | 0.0047 |
| 3318 | 0.0200 |
| 3319 | 0.0534 |
| 3320 | 0.0685 |
| 3321 | 0.0510 |
| 3322 | 0.0368 |
| 3323 | 0.0074 |
| 3324 | 0.0078 |
| 3325 | 0.0124 |
| 3326 | 0.0190 |
| 3327 | 0.0314 |
| 3328 | 0.0043 |
| 3329 | 0.0104 |
| 3330 | 0.0099 |
| 3331 | 0.0294 |
| 3332 | 0.0184 |
| 3333 | 0.0161 |
| 3334 | 0.0122 |
| 3335 | 0.0082 |
| 3336 | 0.0043 |
| 3337 | 0.0176 |
| 3338 | 0.0274 |
| 3339 | 0.0087 |
| 3340 | 0.0087 |
| 3341 | 0.0074 |
| 3342 | 0.0383 |
| 3343 | 0.0662 |
| 3344 | 0.0146 |
| 3345 | 0.0091 |
| 3346 | 0.0109 |
| 3347 | 0.0037 |
| 3348 | 0.0128 |
| 3349 | 0.0085 |
| 3350 | 0.0063 |
| 3351 | 0.0042 |
| 3352 | 0.0265 |
| 3353 | 0.1405 |
| 3354 | 0.0042 |
| 3355 | 0.0081 |
| 3356 | 0.0068 |
| 3357 | 0.0135 |
| 3358 | 0.0024 |
| 3359 | 0.0043 |
| 3360 | 0.0081 |
| 3361 | 0.0187 |
| 3362 | 0.0131 |
| 3363 | 0.0089 |
| 3364 | 0.0134 |
| 3365 | 0.0215 |
| 3366 | 0.0127 |
| 3367 | 0.0101 |
| 3368 | 0.0196 |
| 3369 | 0.1268 |
| 3370 | 0.0126 |
| 3371 | 0.0094 |
| 3372 | 0.0116 |
| 3373 | 0.0274 |
| 3374 | 0.0316 |
| 3375 | 0.0123 |
| 3376 | 0.0148 |
| 3377 | 0.0135 |
| 3378 | 0.0181 |
| 3379 | 0.0095 |
| 3380 | 0.0206 |
| 3381 | 0.0089 |
| 3382 | 0.0108 |
| 3383 | 0.0082 |
| 3384 | 0.0092 |
| 3385 | 0.0165 |
| 3386 | 0.0044 |
| 3387 | 0.0205 |
| 3388 | 0.0206 |
| 3389 | 0.0127 |
| 3390 | 0.0090 |
| 3391 | 0.0088 |
| 3392 | 0.0133 |
| 3393 | 0.0128 |
| 3394 | 0.0069 |
| 3395 | 0.0100 |
| 3396 | 0.0091 |
| 3397 | 0.0074 |
| 3398 | 0.0058 |
| 3399 | 0.0194 |
| 3400 | 0.0087 |
| 3401 | 0.0069 |
| 3402 | 0.0125 |
| 3403 | 0.0107 |
| 3404 | 0.0150 |
| 3405 | 0.0082 |
| 3406 | 0.0373 |
| 3407 | 0.0212 |
| 3408 | 0.0209 |
| 3409 | 0.0156 |
| 3410 | 0.0202 |
| 3411 | 0.0110 |
| 3412 | 0.0164 |
| 3413 | 0.0333 |
| 3414 | 0.0737 |
| 3415 | 0.0736 |
| 3416 | 0.0780 |
| 3417 | 0.0508 |
| 3418 | 0.0362 |
| 3419 | 0.0622 |
| 3420 | 0.0609 |
| 3421 | 0.0336 |
| 3422 | 0.0782 |
| 3423 | 0.1076 |
| 3424 | 0.1333 |
| 3425 | 0.0548 |
| 3426 | 0.0107 |
| 3427 | 0.0168 |
| 3428 | 0.0098 |
| 3429 | 0.0415 |
| 3430 | 0.0076 |
| 3431 | 0.0049 |
| 3432 | 0.0114 |
| 3433 | 0.0406 |
| 3434 | 0.0105 |
| 3435 | 0.0129 |
| 3436 | 0.0170 |
| 3437 | 0.0112 |
| 3438 | 0.0661 |
| 3439 | 0.3332 |
| 3440 | 0.0582 |
| 3441 | 0.0397 |
| 3442 | 0.0164 |
| 3443 | 0.0120 |
| 3444 | 0.0136 |
| 3445 | 0.1603 |
| 3446 | 0.0652 |
| 3447 | 0.0105 |
| 3448 | 0.0910 |
| 3449 | 0.0268 |
| 3450 | 0.0183 |
| 3451 | 0.0184 |
| 3452 | 0.0258 |
| 3453 | 0.0199 |
| 3454 | 0.0162 |
| 3455 | 0.0146 |
| 3456 | 0.0264 |
| 3457 | 0.0374 |
| 3458 | 0.0184 |
| 3459 | 0.0142 |
| 3460 | 0.0170 |
| 3462 | 0.0319 |
| 3463 | 0.0313 |
| 3464 | 0.0121 |
| 3465 | 0.0096 |
| 3466 | 0.0195 |
| 3467 | 0.0330 |
| 3468 | 0.0260 |
| 3469 | 0.0169 |
| 3470 | 0.0248 |
| 3471 | 0.0212 |
| 3472 | 0.0133 |
| 3473 | 0.0151 |
| 3474 | 0.0118 |
| 3475 | 0.0257 |
| 3476 | 0.0091 |
| 3477 | 0.0384 |
| 3478 | 0.0211 |
| 3479 | 0.0596 |
| 3480 | 0.0175 |
| 3481 | 0.0716 |
| 3482 | 0.0075 |
| 3483 | 0.0095 |
| 3484 | 0.0067 |
| 3485 | 0.0164 |
| 3486 | 0.0084 |
| 3487 | 0.0306 |
| 3488 | 0.9997 |
| 3489 | 0.0220 |
| 3490 | 0.0059 |
| 3491 | 0.0114 |
| 3492 | 0.0139 |
| 3493 | 0.0098 |
| 3494 | 0.0091 |
| 3495 | 0.0168 |
| 3496 | 0.0110 |
| 3497 | 0.0036 |
| 3498 | 0.0472 |
| 3499 | 0.0150 |
| 3500 | 0.0052 |
| 3501 | 0.1030 |
| 3502 | 0.0631 |
| 3503 | 0.0382 |
| 3504 | 0.0262 |
| 3505 | 0.0166 |
| 3506 | 0.0151 |
| 3507 | 0.0397 |
| 3508 | 0.0144 |
| 3509 | 0.1357 |
| 3510 | 0.0553 |
| 3511 | 0.0217 |
| 3512 | 0.0182 |
| 3513 | 0.0108 |
| 3514 | 0.0842 |
| 3515 | 0.1690 |
| 3516 | 0.0103 |
| 3517 | 0.0279 |
| 3518 | 0.0357 |
| 3519 | 0.0104 |
| 3520 | 0.6080 |
| 3521 | 0.0979 |
| 3522 | 0.0408 |
| 3523 | 0.1208 |
| 3524 | 0.0408 |
| 3525 | 0.3459 |
| 3526 | 0.1116 |
| 3527 | 0.0514 |
| 3528 | 0.0878 |
| 3529 | 0.0781 |
| 3530 | 0.0073 |
| 3531 | 0.0486 |
| 3532 | 0.0075 |
| 3533 | 0.0051 |
| 3534 | 0.0226 |
| 3535 | 0.0134 |
| 3536 | 0.0794 |
| 3537 | 0.0299 |
| 3538 | 0.0263 |
| 3539 | 0.0399 |
| 3540 | 0.0109 |
| 3541 | 0.0100 |
| 3542 | 0.0382 |
| 3543 | 0.0133 |
| 3544 | 0.0104 |
| 3545 | 0.1547 |
| 3546 | 0.0198 |
| 3547 | 0.0740 |
| 3548 | 0.0111 |
| 3549 | 0.0370 |
| 3550 | 0.0762 |
| 3551 | 0.0334 |
| 3552 | 0.0205 |
| 3553 | 0.0373 |
| 3554 | 0.0184 |
| 3555 | 0.0428 |
| 3556 | 0.0132 |
| 3557 | 0.0066 |
| 3558 | 0.0298 |
| 3559 | 0.0141 |
| 3560 | 0.0179 |
| 3561 | 0.0405 |
| 3562 | 0.0284 |
| 3563 | 0.0439 |
| 3564 | 0.0195 |
| 3565 | 0.0394 |
| 3566 | 0.0192 |
| 3567 | 0.0335 |
| 3568 | 0.0151 |
| 3569 | 0.0236 |
| 3570 | 0.0163 |
| 3571 | 0.0169 |
| 3572 | 0.0261 |
| 3573 | 0.0124 |
| 3574 | 0.0270 |
| 3575 | 0.0323 |
| 3576 | 0.0173 |
| 3577 | 0.0311 |
| 3578 | 0.0215 |
| 3579 | 0.0182 |
| 3580 | 0.0168 |
| 3581 | 0.0162 |
| 3582 | 0.0710 |
| 3583 | 0.0205 |
| 3584 | 0.0382 |
| 3585 | 0.0308 |
| 3586 | 0.0301 |
| 3587 | 0.0335 |
| 3588 | 0.0167 |
| 3589 | 0.0278 |
| 3590 | 0.0065 |
| 3591 | 0.0239 |
| 3592 | 0.0086 |
| 3593 | 0.3631 |
| 3594 | 0.0162 |
| 3595 | 0.1508 |
| 3596 | 0.1615 |
| 3597 | 0.0231 |
| 3598 | 0.0138 |
| 3599 | 0.0049 |
| 3600 | 0.0401 |
| 3601 | 0.0380 |
| 3602 | 0.0231 |
| 3603 | 0.0092 |
| 3604 | 0.0068 |
| 3605 | 0.0116 |
| 3606 | 0.0130 |
| 3607 | 0.0118 |
| 3608 | 0.0527 |
| 3609 | 0.0188 |
| 3610 | 0.0101 |
| 3611 | 0.0371 |
| 3612 | 0.0136 |
| 3613 | 0.0241 |
| 3614 | 0.0097 |
| 3615 | 0.0267 |
| 3616 | 0.0183 |
| 3617 | 0.0788 |
| 3618 | 0.0218 |
| 3619 | 0.0134 |
| 3620 | 0.0372 |
| 3621 | 0.0243 |
| 3622 | 0.0183 |
| 3623 | 0.0623 |
| 3624 | 0.0215 |
| 3625 | 0.0233 |
| 3626 | 0.0073 |
| 3627 | 0.0367 |
| 3628 | 0.0230 |
| 3629 | 0.0084 |
| 3630 | 0.0067 |
| 3631 | 0.0160 |
| 3632 | 0.0087 |
| 3633 | 0.0086 |
| 3634 | 0.0063 |
| 3635 | 0.0349 |
| 3636 | 0.0209 |
| 3637 | 0.0291 |
| 3638 | 0.0709 |
| 3639 | 0.0105 |
| 3640 | 0.0110 |
| 3641 | 0.0074 |
| 3642 | 0.0068 |
| 3643 | 0.0227 |
| 3644 | 0.0188 |
| 3645 | 0.0266 |
| 3646 | 0.0947 |
| 3647 | 0.0277 |
| 3648 | 0.0215 |
| 3649 | 0.0075 |
| 3650 | 0.0175 |
| 3651 | 0.0241 |
| 3652 | 0.0221 |
| 3653 | 0.0390 |
| 3654 | 0.0451 |
| 3655 | 0.0618 |
| 3656 | 0.1783 |
| 3657 | 0.1211 |
| 3658 | 0.0880 |
| 3659 | 0.0476 |
| 3660 | 0.0679 |
| 3661 | 0.0358 |
| 3662 | 0.0151 |
| 3663 | 0.0153 |
| 3664 | 0.0277 |
| 3665 | 0.0232 |
| 3666 | 0.0320 |
| 3667 | 0.0374 |
| 3668 | 0.0783 |
| 3669 | 0.1524 |
| 3670 | 0.0642 |
| 3671 | 0.1050 |
| 3672 | 0.0207 |
| 3673 | 0.0292 |
| 3674 | 0.0134 |
| 3675 | 0.0193 |
| 3676 | 0.0092 |
| 3677 | 0.0113 |
| 3678 | 0.0092 |
| 3679 | 0.0096 |
| 3680 | 0.0158 |
| 3681 | 0.0717 |
| 3682 | 0.0514 |
| 3683 | 0.1180 |
| 3684 | 0.0771 |
| 3685 | 0.0302 |
| 3686 | 0.0276 |
| 3687 | 0.0199 |
| 3688 | 0.0173 |
| 3689 | 0.0299 |
| 3690 | 0.0388 |
| 3691 | 0.0713 |
| 3692 | 0.0584 |
| 3693 | 0.0598 |
| 3694 | 0.0728 |
| 3695 | 0.6288 |
| 3696 | 0.0151 |
| 3697 | 0.0112 |
| 3698 | 0.0156 |
| 3699 | 0.0969 |
| 3700 | 0.0567 |
| 3701 | 0.0922 |
| 3702 | 0.0625 |
| 3703 | 0.0234 |
| 3704 | 0.0219 |
| 3705 | 0.0242 |
| 3706 | 0.0703 |
| 3707 | 0.0141 |
| 3708 | 0.0114 |
| 3709 | 0.0545 |
| 3710 | 0.0822 |
| 3711 | 0.0915 |
| 3712 | 0.1291 |
| 3713 | 0.0222 |
| 3714 | 0.0124 |
| 3715 | 0.2345 |
| 3716 | 0.1651 |
| 3717 | 0.0050 |
| 3718 | 0.0088 |
| 3719 | 0.0298 |
| 3720 | 0.0117 |
| 3721 | 0.0184 |
| 3722 | 0.0412 |
| 3723 | 0.0841 |
| 3724 | 0.0739 |
| 3725 | 0.0305 |
| 3726 | 0.0131 |
| 3727 | 0.0504 |
| 3728 | 0.0256 |
| 3729 | 0.0303 |
| 3730 | 0.0228 |
| 3731 | 0.0169 |
| 3732 | 0.0562 |
| 3733 | 0.1147 |
| 3734 | 0.1280 |
| 3735 | 0.0206 |
| 3736 | 0.0185 |
| 3737 | 0.0105 |
| 3738 | 0.0207 |
| 3739 | 0.0405 |
| 3740 | 0.0161 |
| 3741 | 0.0088 |
| 3742 | 0.0017 |
| 3743 | 0.0048 |
| 3744 | 0.0166 |
| 3745 | 0.0150 |
| 3746 | 0.0068 |
| 3747 | 0.0121 |
| 3748 | 0.0105 |
| 3749 | 0.0566 |
| 3750 | 0.1251 |
| 3751 | 0.0096 |
| 3752 | 0.1164 |
| 3753 | 0.0060 |
| 3754 | 0.2498 |
| 3755 | 0.3142 |
| 3756 | 0.0035 |
| 3757 | 0.0026 |
| 3758 | 0.0190 |
| 3759 | 0.0392 |
| 3760 | 0.0047 |
| 3761 | 0.0368 |
| 3762 | 0.0275 |
| 3763 | 0.0074 |
| 3764 | 0.2385 |
| 3765 | 0.3438 |
| 3766 | 0.0312 |
| 3767 | 0.0096 |
| 3768 | 0.0065 |
| 3769 | 0.0035 |
| 3770 | 0.2391 |
| 3771 | 0.0047 |
| 3772 | 0.0026 |
| 3773 | 0.0010 |
| 3774 | 0.0063 |
| 3775 | 0.0058 |
| 3776 | 0.0036 |
| 3777 | 0.0034 |
| 3778 | 0.0266 |
| 3779 | 0.0017 |
| 3780 | 0.0120 |
| 3781 | 0.0044 |
| 3782 | 0.0108 |
| 3783 | 0.0292 |
| 3784 | 0.0325 |
| 3785 | 0.0049 |
| 3786 | 0.0060 |
| 3787 | 0.0083 |
| 3788 | 0.0070 |
| 3789 | 0.0020 |
| 3790 | 0.0112 |
| 3791 | 0.0087 |
| 3792 | 0.0509 |
| 3793 | 0.0039 |
| 3794 | 0.0038 |
| 3795 | 0.0058 |
| 3796 | 0.0019 |
| 3797 | 0.0021 |
| 3798 | 0.0284 |
| 3799 | 0.0111 |
| 3800 | 0.0121 |
| 3801 | 0.0025 |
| 3802 | 0.0366 |
| 3803 | 0.0027 |
| 3804 | 0.0002 |
| 3805 | 0.0012 |
| 3806 | 0.0052 |
| 3807 | 0.0204 |
| 3808 | 0.5693 |
| 3809 | 0.0154 |
| 3810 | 0.0198 |
| 3811 | 0.0039 |
| 3812 | 0.0133 |
| 3813 | 0.0069 |
| 3814 | 0.0534 |
| 3815 | 0.0217 |
| 3816 | 0.0260 |
| 3817 | 0.0576 |
| 3818 | 0.0039 |
| 3819 | 0.0204 |
| 3820 | 0.0068 |
| 3821 | 0.0040 |
| 3822 | 0.0209 |
| 3823 | 0.0404 |
| 3824 | 0.0034 |
| 3825 | 0.0197 |
| 3826 | 0.0725 |
| 3827 | 0.1408 |
| 3828 | 0.2164 |
| 3829 | 0.0192 |
| 3830 | 0.1995 |
| 3831 | 0.0225 |
| 3832 | 0.0455 |
| 3833 | 0.0828 |
| 3834 | 0.0241 |
| 3835 | 0.0283 |
| 3836 | 0.0456 |
| 3837 | 0.0174 |
| 3838 | 0.0187 |
| 3839 | 0.1783 |
| 3840 | 0.3402 |
| 3841 | 0.0341 |
| 3842 | 0.0161 |
| 3843 | 0.0407 |
| 3844 | 0.0152 |
| 3845 | 0.6643 |
| 3846 | 0.1133 |
| 3847 | 0.1375 |
| 3848 | 0.3171 |
| 3849 | 0.2496 |
| 3850 | 0.2817 |
| 3851 | 0.1254 |
| 3852 | 0.1420 |
| 3853 | 0.1035 |
| 3854 | 0.0452 |
| 3855 | 0.1194 |
| 3856 | 0.0942 |
| 3857 | 0.1657 |
| 3858 | 0.2977 |
| 3859 | 0.0527 |
| 3860 | 0.0739 |
| 3861 | 0.3484 |
| 3862 | 0.3678 |
| 3863 | 0.4300 |
| 3864 | 0.1300 |
| 3865 | 0.1190 |
| 3866 | 0.6765 |
| 3867 | 0.0481 |
| 3868 | 0.1560 |
| 3869 | 0.0711 |
| 3870 | 0.0280 |
| 3871 | 0.0457 |
| 3872 | 0.1736 |
| 3873 | 0.0705 |
| 3874 | 0.8148 |
| 3875 | 0.1472 |
| 3876 | 0.1445 |
| 3877 | 0.0263 |
| 3878 | 0.0215 |
| 3879 | 0.0620 |
| 3880 | 0.0119 |
| 3881 | 0.0245 |
| 3882 | 0.0268 |
| 3883 | 0.0241 |
| 3884 | 0.1467 |
| 3885 | 0.1172 |
| 3886 | 0.2016 |
| 3887 | 0.0563 |
| 3888 | 0.1165 |
| 3889 | 0.2718 |
| 3890 | 0.4338 |
| 3891 | 0.2347 |
| 3892 | 0.3881 |
| 3893 | 0.3688 |
| 3894 | 0.1057 |
| 3895 | 0.0494 |
| 3896 | 0.2243 |
| 3897 | 0.0636 |
| 3898 | 0.0238 |
| 3899 | 0.0274 |
| 3900 | 0.1853 |
| 3901 | 0.1729 |
| 3902 | 0.0672 |
| 3903 | 0.0497 |
| 3904 | 0.0381 |
| 3905 | 0.0384 |
| 3906 | 0.0177 |
| 3907 | 0.0166 |
| 3908 | 0.1250 |
| 3909 | 0.0157 |
| 3910 | 0.5972 |
| 3911 | 0.0905 |
| 3912 | 0.2517 |

The compounds of formula (I) possess activity as inhibitors of the PD-1/PD-L1 interaction, and therefore, can be used in the treatment of diseases or deficiencies associated with the PD-1/PD-L1 interaction. Via inhibition of the PD-1/PD-L1 interaction, the compounds of the present disclosure can be employed to treat infectious diseases such as HIV, septic shock, Hepatitis A, B, C, or D and cancer.

It is to be appreciated that the Detailed Description section, and not the Summary and Abstract sections, is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

1. A compound of formula (I): 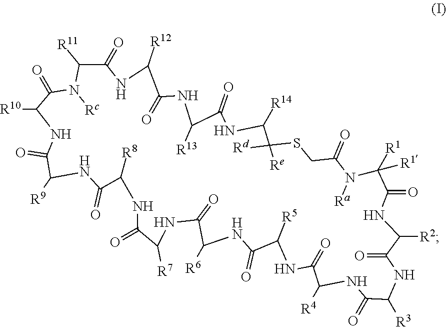
or a pharmaceutically acceptable salt thereof, wherein:
R¹ is selected from C₁-C₆alkyl, C₁-C₂alkylaminoC₁-C₆alkyl, C₁-C₆alkylcarbonylaminoC₁-C₆alkyl, C₁-C₆alkylheteroarylC₁-C₆alkyl, C₁-C₆alkylimidazolylC₁-C₂alkyl, aminoC₁-C₆alkyl, aminocarbonylC₁-C₆alkyl, aminocarbonylaminoC₁-C₆alkyl, arylC₁-C₂alkyl, biarylC₁-C₆alkyl optionally substituted with carboxy, guanidinylC₂-C₆alkyl, carboxyC₁-C₆alkyl, C₃-C₆cycloalkyl, (C₃-C₆cycloalkyl) C₁-C₆alkyl, C₃-C₆cycloalkylcarbonylaminoC₁-C₆alkyl, fluoroheterocyclylC₁-C₆alkyl, heterocyclylC₁-C₆alkyl, heteroarylcarbonylaminoC₁-C₆alkyl, heteroarylC₁-C₆alkyl, hydroxyC₁-C₆alkyl, and methoxyC₁-C₂alkyl; wherein the aryl part of the arylC₁-C₂alkyl is optionally substituted with one, two, or three groups independently selected from amino, aminocarbonyl, carboxy, carboxyC₁-C₆alkyl, carboxymethoxy, cyano, fluoro, hydroxy, methoxy, methyl, methylcarbonylamino, and trifluoromethyl;
R^{1′} is hydrogen, or, R¹ and R^{1′}, together with the carbon atom to which they are attached, form a cyclopropyl ring;
R² is selected from arylC₁-C₂alkyl, carboxyC₁-C₆alkyl, (C₃-C₆cycloalkyl) C₁-C₆alkyl, guanidinylC₂-C₆alkyl, heteroarylC₁-C₆alkyl, heterocyclylC₁-C₆alkyl, and hydroxyC₁-C₆alkyl, wherein the aryl part of the arylC₁-C₂alkyl is optionally substituted with one, two, or three groups independently selected from aminocarbonyl, carboxy, carboxyC₁-C₆alkoxy, cyano, fluoro, hydroxy, methyl, methoxy, —SO₃H, and trifluoromethoxy;
R³ is carboxymethyl;
R⁴ is selected from arylC₁-C₂alkyl, benzothienylC₁-C₂alkyl, heteroarylC₁-C₆alkyl, indolylC₁-C₆alkyl, and naphthalenylC₁-C₂alkyl; wherein the aryl part of the arylC₁-C₂alkyl is optionally substituted with one, two, three, four, or five groups independently selected from amino, carboxyC₁-C₆alkyl, chloro, cyano, fluoro, hydroxy, methoxy, methyl, and trifluoromethyl;
R⁵ is selected from C₁-C₆alkyl, C₁-C₆alkylcarbonylaminoC₁-C₆alkyl, aminocarbonylaminoC₂-C₆alkyl, aryl, arylC₁-C₂alkyl, benzothienylC₁-C₂alkyl, carboxyC₁-C₆alkyl, C₃-C₆cycloalkyl, (C₃-C₆cycloalkyl) C₁-C₆alkyl, C₃-C₆cycloalkylcarbonylaminoC₁-C₆alkyl, fluoroC₁-C₆alkyl, heteroarylC₁-C₆alkyl, heterocycloalkylC₁-C₆alkyl, and hydroxyC₁-C₆alkyl; wherein the aryl part of the arylC₁-C₂alkyl is optionally substituted with one, two, three, four, or five groups independently selected from amino, aminocarbonyl, aminomethyl, carboxy, carboxyC₁-C₆alkyl, carboxymethoxy, cyano, fluoro, hydroxy, methoxy, methyl, methylcarbonylamino, trifluoromethoxy, and trifluoromethyl;
R⁶ is biarylC₁-C₆alkyl;
R⁷ is selected from fluoroC₁-C₆alkylcarbonylaminoC₁-C₆alkyl, C₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, arylC₁-C₂alkyl, carboxyC₁-C₆alkyl, (C₃-C₆cycloalkyl) C₁-C₆alkyl, fluoroC₁-C₆alkyl, guanidinylC₂-C₄alkyl, heteroarylC₁-C₆alkyl, C₃-C₆heterocycloalkylC₁-C₆alkyl, hydroxyC₁-C₆alkyl, and methylsulfanylC₁-C₆alkyl; wherein the aryl part of the arylC₁-C₂alkyl is optionally substituted with one, two, three, four, or five groups independently selected from amino, aminocarbonyl, aminoC₁-C₆alkyl, aminocarbonyl, aminomethyl, carboxy, carboxyC₁-C₆alkyl, chloro, fluoro, methyl, hydroxy, methylcarbonylamino, and trifluoromethyl;
R⁸ is selected from C₁-C₂alkylcarbonylaminoC₁-C₆alkyl, aminoC₁-C₆alkyl, aminocarbonylC₁-C₆alkyl, aminocarbonylaminoC₁-C₆alkyl, carboxyC₁-C₆alkyl, guanidinylC₁-C₆alkyl, and heteroarylC₁-C₆alkyl;
R⁹ is C₁-C₆alkyl or (C₃-C₆cycloalkyl) C₁-C₂alkyl;
R¹⁰ is selected from aminoC₁-C₆alkyl, carboxyC₁-C₆alkyl, hydroxyC₁-C₆alkyl, aminocarbonylC₁-C₂alkyl, guanidinylC₁-C₆alkyl, and heteroarylC₁-C₆alkyl;
R¹¹ is selected from C₂-C₆alkyl, arylC₁-C₂alkyl, and (C₃-C₆cycloalkyl) C₁-C₂alkyl; wherein the aryl part of the arylC₁-C₂alkyl is optionally substituted with one, two, three, four, or five groups independently selected from chloro, fluoro, methyl, and trifluoromethyl;
R¹² is selected from C₃-C₆alkyl, aminoC₁-C₆alkyl, fluoroC₄-C₆alkyl, heteroarylC₁-C₆alkyl, hydroxyC₁-C₆alkyl, and hydroxyarylC₁-C₂alkyl;
R¹³ is selected from C₁-C₆alkyl, C₁-C₆alkylcarbonylaminoC₁-C₆alkyl, aminoC₁-C₆alkyl, aminocarbonylC₁-C₆alkyl, aminocarbonylaminoC₁-C₆alkyl, aryl, arylC₁-C₂alkyl, carboxyC₁-C₆alkyl, cyanoC₁-C₆alkyl, guanidinylC₁-C₆alkyl, C₂-C₆alkynylmethoxy, heteroaryl, heteroarylC₁-C₆alkyl, C₃-C₆heterocyclylC₁-C₆alkyl, hydroxyC₁-C₆alkyl, and hydroxyarylC₁-C₂alkyl; wherein the aryl part of the arylC₁-C₂alkyl is optionally substituted with one, two, three, four, or five groups independently selected from carboxyC₁-C₆alkyl, ethynylmethoxy, and hydroxy;
R¹⁴ is —C(O) NR^{14′}CR¹⁵R^{15′}R^{15″}, —C(O)NH(CH₂)_{o′}Ph(CH₂)_{o′}C(O)NHCHR¹⁷R^{17′}, —C(O)NH(CH₂)_{o′}cyclopropyl(CH₂)_{o′}C(O)NHCHR¹⁷R^{17′}, or —C(O)NR⁵⁰R⁵¹, wherein:
R⁵⁰ and R⁵¹, together with the nitrogen atom to which they are attached, form a piperazine ring, wherein the ring is further substituted with one —(CH₂)_{o′}C(O)NHCHR¹⁷R^{17′} group;
o′ is 0, 1, or 2;
R^{14′} is hydrogen or C₁-C₆alkyl, or R¹⁵ and R^{14′}, together with the atoms to which they are attached, form a morpholine, piperazine, or piperidine ring;
R¹⁵ is selected from hydrogen, C₂-C₆alkenyl, C₁-C₁₆alkyl, C₁-C₆alkylcarbonylaminoC₁-C₆alkyl, C₂-C₆alkynyl, aminoC₁-C₆alkyl, aminocarbonylC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, and hydroxyC₁-C₆alkyl;
R^{15′} is hydrogen or R¹⁵ and R^{15′}, together with the atoms to which they are attached, form a C₃-C₈cycloalkyl ring; and
R^{15″} is —(CH₂)ₘCO₂H CH₂O((CH₂)₂O)ₙCH₂C(O)NHCHR¹⁶R^{16′} or —C(O)NHCHR¹⁶R^{16′}; wherein:
R¹⁶ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{16′} is —(CH₂)ₘCO₂H, —CH₂O((CH₂)₂O)ₙCH₂C(O)NR⁷⁵CR^{17″}R¹⁷R^{17′}, -Ph(CH₂)_{o′}C(O)NHCHR¹⁷R^{17′}, or —(CH₂)_{o′}C(O)NHCHR¹⁷R¹⁷; wherein:
R⁷⁵ is hydrogen;
R¹⁷ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; or R¹⁷ and R⁷⁵, together with the atoms to which they are attached, form a pyrrolidine ring;
R^{17′} is —CH₂O((CH₂)₂O)ₙCH₂C(O)NHCHR¹⁸R^{18′}, —(CH₂)ₘCO₂H or —(CH₂)ₘC(O)NHR¹⁸R^{18′}; and
R^{17″} is hydrogen, or R^{17″} and R¹⁷ form a C₃-C₈ cycloalkyl ring; wherein:
R¹⁸ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{18′} is —(CH₂)ₘCO₂H, —(CH₂)ₘC(O)NR¹⁹R^{19′}, or —CH₂O((CH₂)₂O)ₙCH₂C(O)NHCHR¹⁹R^{19′}; wherein:
R¹⁹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl;
R^{19′} is —(CH₂)ₘC(O)NR¹⁹R^{19′}, —(CH₂)ₘCO₂H, or —CH₂O((CH₂)₂O)ₙCH₂C(O)NHCHR²⁰R^{20′}, wherein:
R²⁰ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{20′} is —(CH₂)ₘCO₂H or —(CH₂)ₘC(O)NR²¹R^{21′}; wherein:
R²¹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{21′} is —(CH₂)ₘCO₂H or —(CH₂)ₘC(O)NR²²R^{22′}; wherein:
R²² is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R²² is —(CH₂)ₘCO₂H; wherein
m is a integer from 1 to 10;
n is 1, 2, or 3; and
o′ is 0, 1, or 2;
R^{a} is hydrogen or C₁-C₆alkyl;
R^{c} is hydrogen or C₁-C₆alkyl;
R^{d} is hydrogen or C₁-C₆alkyl; and
R^{e} is hydrogen or C₁-C₆alkyl.

2. (canceled)

3. The compound of claim 1, or the pharmaceutically acceptable salt thereof, wherein R¹ is C₁-C₄alkyl, aminoC₁-C₃alkyl, aminocarbonylmethyl, benzyl, guanidinylpropyl, hydroxyC₁-C₃alkyl, imidazolylmethyl, methoxymethyl, morpholinylmethyl, and pyridinylmethyl; wherein the benzyl is optionally ring substituted with one, two, or three groups independently selected from aminocarbonyl, carboxy, carboxymethoxy, cyano, fluoro, and trifluoromethyl.

4. The compound of claim 1, or the pharmaceutically acceptable salt thereof, wherein R² is arylC₁-C₂alkyl, optionally ring substituted with one, two, or three groups independently selected from aminocarbonyl, carboxy, carboxyC₁-C₆alkoxy, cyano, fluoro, methyl, hydroxy, —SO₃H, and trifluoromethoxy.

5. (canceled)

6. The compound of claim 1, or the pharmaceutically acceptable salt thereof, wherein R³ is carboxymethyl.

7. The compound of claim 1, or the pharmaceutically acceptable salt thereof, wherein R⁴ is selected from arylC₁-C₂alkyl, heteroarylC₁-C₆alkyl, and indolylC₁-C₂alkyl; wherein the aryl part of the arylC₁-C₂alkyl are optionally substituted with one or more groups independently selected from amino, carboxyC₁-C₆alkyl, chloro, cyano, fluoro, hydroxy, methoxy, methyl, and trifluoromethyl.

8. (canceled)

9. The compound of claim 1, or the pharmaceutically acceptable salt thereof, wherein R⁵ is C₁-C₆alkyl or arylC₁-C₂alkyl; wherein the aryl part of the arylC₁-C₂alkyl is optionally substituted with one, two, three, four, or five groups independently selected from amino, aminocarbonyl, aminomethyl, cyano, carboxy, carboxyC₁-C₆alkyl, carboxymethoxy, fluoro, hydroxy, methoxy, methyl, methylcarbonylamino, trifluoromethyl, and trifluoromethoxy.

10. (canceled)

11. The compound of claim 1, or the pharmaceutically acceptable salt thereof, wherein R⁶ is biarylC₁-C₆alkyl.

12. The compound of claim 1, or the pharmaceutically acceptable salt thereof, wherein R₇ is selected from C₄-C₆alkyl, C₁-C₆alkylcarbonylaminoC₁-C₆alkyl, aminocarbonylC₁-C₆alkyl, aminocarbonylaminoC₁-C₆alkyl, and arylC₁-C₂alkyl; wherein the aryl part of the arylC₁-C₂alkyl is optionally substituted with one, two, three, four, or five groups independently selected from amino, chloro, fluoro, hydroxy, methyl, aminocarbonyl, aminoC₁-C₆alkyl, aminocarbonyl, aminomethyl, methylcarbonylamine, carboxy, hydroxy, and carboxyC₁-C₆alkyl.

13. (canceled)

14. The compound of claim 1, or the pharmaceutically acceptable salt thereof, wherein R⁸ is aminoC₁-C₆alkyl or heteroarylC₁-C₆alkyl.

15. (canceled)

16. The compound of claim 1, or the pharmaceutically acceptable salt thereof, wherein R⁹ is C₁-C₆alkyl and the stereochemistry of the center to which R⁹ is attached is R-stereochemistry.

17. (canceled)

18. The compound of claim 1, or the pharmaceutically acceptable salt thereof, wherein R¹⁰ is aminoC₁-C₆alkyl or heteroarylC₁-C₆alkyl.

19. (canceled)

20. The compound of claim 1, or the pharmaceutically acceptable salt thereof, wherein R¹¹ is (C₃-C₆cycloalkyl) C₁-C₂alkyl.

21. (canceled)

22. The compound of claim 1, or the pharmaceutically acceptable salt thereof, wherein R¹² is selected from C₄-C₆alkyl, fluoroC₄-C₆alkyl, hydroxyC₁-C₆alkyl, and hydroxyarylC₁-C₂alkyl.

23. (canceled)

24. The compound of claim 1, or the pharmaceutically acceptable salt thereof, wherein R¹³ is selected from C₁-C₆alkyl, aminoC₁-C₆alkyl, aminocarbonylC₁-C₆alkyl, aminocarbonylaminoC₁-C₆alkyl, carboxyC₁-C₆alkyl, guanidinylC₁-C₆alkyl, heteroaryl, heteroarylC₁-C₆alkyl, and hydroxyC₁-C₆alkyl.

25.-27. (canceled)

28. The compound of claim 1, or the pharmaceutically acceptable salt thereof, wherein R¹ is selected from methyl, ethyl, propyl, butyl, hydroxyethyl, aminomethyl, aminoethyl, aminocarbonylmethyl, methoxymethyl, guanidinylbutyl, guanidinylpropyl, imidazolylmethyl, pyridinylmethyl, morpholinylmethyl, cyclobutylmethyl, cyclohexylmethyl, and arylmethyl; wherein the aryl part of the arylmethyl is optionally substituted with one, two, or three groups independently selected from aminocarbonyl, carboxy, carboxymethoxy, cyano, fluoro, and trifluoromethyl;
R² is arylmethyl; wherein the aryl part of the arylmethyl is optionally substituted with one, two, or three groups independently selected from carboxy, carboxyC₁-C₆alkoxy, cyano, and hydroxy;
R³ is carboxymethyl;
R⁴ is selected from arylmethyl, heteroarylmethyl, and indolylmethyl; wherein the aryl part of the arylmethyl are optionally substituted with one or more groups independently selected from methyl and trifluoromethyl;
R⁵ is arylmethyl or isopropyl; wherein the aryl part of the arylmethyl is optionally substituted with one, two, three, four, or five groups independently selected from amino, aminocarbonyl, carboxy, carboxymethoxy, cyano, fluoro, hydroxy, methoxy, methyl, and trifluoromethyl;
R⁶ is biarylC₁-C₃alkyl;
R⁷ is selected from isobutyl, isopropyl, methylcarbonylaminobutyl, aminocarbonylethyl, aminocarbonylaminopropyl, and arylmethyl wherein the aryl part of the arylmethyl is optionally substituted with one, two, three, four, or five groups independently selected from carboxyl, carboxyalkyl, carboxymethoxy, hydroxy, and trifluoromethyl;
R⁸ is selected from aminobutyl, aminoethyl, aminopropyl, and imidazolylmethyl;
R⁹ is isopropylmethyl, and the stereochemistry of the center to which R⁹ is attached is R-stereochemistry;
R¹⁰ is aminomethyl, aminoethyl or imidazolylmethyl;
R¹¹ is C₆cycloalkylmethyl;
R¹² is selected from hydroxyethyl, hydroxyisopropyl, fluoroisopropyl, isopropyl,
R¹³ is selected from aminobutyl, aminocarbonylaminopropyl, aminocarboxyethyl, aminoethyl, aminomethyl, aminopropyl, carboxyethyl, carboxymethyl, carboxypropyl, guanidinylpropyl, hydroxyethyl, hydroxymethyl, and imidazolylmethyl;
R¹⁴ is —C(O)NR^{14′}CR¹⁵R^{15′}R^{15″}, —C(O)NH(CH₂)_{o′}Ph(CH₂)_{o′}C(O)NHCHR¹⁷R^{17′}, —C(O)NH(CH₂)_{o′}cyclopropyl(CH₂)_{o′}C(O)NHCHR¹⁷R^{17′}, or —C(O)NR⁵⁰R⁵¹, wherein:
R⁵⁰ and R⁵¹, together with the nitrogen atom to which they are attached, form a piperazine ring, wherein the ring is further substituted with one —(CH₂)_{o′}C(O)NHCHR¹⁷R^{17′} group;
o′ is 0, 1, or 2;
R^{14′} is hydrogen or C₁-C₆alkyl, or R¹⁵ and R^{14′}, together with the atoms to which they are attached, form a morpholine, piperazine, or piperidine ring;
R¹⁵ is selected from hydrogen, C₂-C₆alkenyl, C₁-C₁₆alkyl, C₁-C₆alkylcarbonylaminoC₁-C₆alkyl, C₂-C₆alkynyl, aminoC₁-C₆alkyl, aminocarbonylC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, and hydroxyC₁-C₆alkyl;
R^{15′} is hydrogen or R¹⁵ and R^{15′}, together with the atoms to which they are attached, form a C₃-C₈cycloalkyl ring; and
R^{15″} is —(CH₂)ₘCO₂H CH₂O((CH₂)₂O)ₙCH₂C(O)NHCHR¹⁶R^{16′}, or —C(O)NHCHR¹⁶R^{16′}; wherein:
m is a integer from 1 to 10;
R¹⁶ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{16′} is —(CH₂)ₘCO₂H, —CH₂O((CH₂)₂O)ₙCH₂C(O)NR⁷⁵CR^{17″}R¹⁷R^{17′}, -Ph(CH₂)_{o′}C(O)NHCHR¹⁷R^{17′} or —(CH₂)_{o′}C(O)NHCHR¹⁷R^{17′}; wherein:
R⁷⁵ is hydrogen;
R¹⁷ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; or R¹⁷ and R⁷⁵, together with the atoms to which they are attached, form a pyrrolidine ring;
R^{17′} is —CH₂O((CH₂)₂O)ₙCH₂C(O)NHCHR¹⁸R^{18′}, —(CH₂)ₘCO₂H or —(CH₂)ₘC(O)NHR¹⁸R^{18′}; and
R^{17″} is hydrogen, or R^{17″} and R¹⁷ form a C₃-C₈ cycloalkyl ring; wherein:
R¹⁸ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{18′} is —(CH₂)ₘCO₂H, —(CH₂)ₘC(O)NR¹⁹R^{19′}, or —CH₂O((CH₂)₂O)ₙCH₂C(O)NHCHR¹⁹R^{19′}; wherein:
R¹⁹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl;
R^{19′} is —(CH₂)ₘC(O)NR¹⁹R^{19′}, —(CH₂)ₘCO₂H, or —CH₂O((CH₂)₂O)ₙCH₂C(O)NHCHR²⁰R^{20′}; wherein:
R²⁰ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{20′} is —(CH₂)ₘCO₂H or —(CH₂)ₘC(O)NR²¹R^{21′}; wherein:
R²¹ is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{21′} is —(CH₂)ₘCO₂H or —(CH₂)ₘC(O)NR²²R^{22′}; wherein:
R²² is hydrogen, C₁-C₁₆alkyl, C₂-C₆alkynyl, aminoC₁-C₆alkyl, aminocarbonylaminoC₁-C₆alkyl, carboxy, carboxyC₁-C₆alkyl, guanidinylC₁-C₆alkyl, heteroaryl, heteroarylC₁-C₆alkyl, heterocyclyl, heterocyclylC₁-C₆alkyl, or hydroxyC₁-C₆alkyl; and
R^{22′} is —(CH₂)ₘCO₂H; wherein:
m is a integer from 1 to 10;
n is 1, 2, or 3;
o′ is 0, 1, or 2;
R^{a} is hydrogen;
R^{c} is hydrogen or C₁-C₆alkyl;
R^{d} is hydrogen or C₁-C₆alkyl; and
R^{e} is hydrogen or C₁-C₆alkyl.

29. The compound of claim 1, or the pharmaceutically acceptable salt thereof, wherein the compound is one of compounds 1000 to 3912 listed in Table 3.

30. A pharmaceutical composition comprising a compound of claim 1, or a pharmaceutically acceptable salt thereof.

31. A method of enhancing, stimulating, and/or increasing an immune response in a subject in need thereof, wherein the method comprises administering to the subject a therapeutically effective amount of a compound of claim 1, or a pharmaceutically acceptable salt thereof.

32. A method of blocking the interaction of PD-1 with PD-L1 in a subject, wherein the method comprises administering to the subject a therapeutically effective amount of a compound of claim 1, or a pharmaceutically acceptable salt thereof.
